(19) United States
(12) Patent Application Publication
(10) Pub. No.: US 2025/0034170 A1
(43) Pub. Date: Jan. 30, 2025

(54) SMALL MOLECULE MODULATORS OF GLUCOCEREBROSIDASE ACTIVITY AND USES THEREOF

(71) Applicant: Vanqua Bio, Inc., Chicago, IL (US)

(72) Inventors: Kevin Hunt, Chicago, IL (US); Jianbin Zheng, Chicago, IL (US); Sida Shen, Chicago, IL (US); Jeremiah Brittin, Chicago, IL (US); Zhiquan Lei, Chicago, IL (US); Greg Sheyka, Chicago, IL (US)

(73) Assignee: Vanqua Bio, Inc., Chicago, IL (US)

(21) Appl. No.: 18/700,625

(22) PCT Filed: Oct. 12, 2022
(86) PCT No.: PCT/US2022/046386

§ 371 Date: Apr. 11, 2024

Related U.S. Application Data
(60) Provisional application No. 63/255,272, filed on Oct. 13, 2021.

(51) Int. Cl. C07D 519/00; A61K 31/438; A61K 31/444; A61K 31/497; A61K 31/4985; A61K 31/506; A61K 31/519; A61K 31/5377; A61K 31/5386; C07D 401/14; C07D 417/14; C07D 471/10; C07D 487/04

(52) U.S. Cl. CPC C07D 519/00; A61K 31/438; A61K 31/444; A61K 31/497; A61K 31/4985; A61K 31/506; A61K 31/519; A61K 31/5377; A61K 31/5386; C07D 401/14; C07D 417/14; C07D 471/10; C07D 487/04

(57) ABSTRACT

Provided herein are compounds that modulate glucocerebrosidase (GCase), an enzyme whose activity is associated with neurological diseases and disorders (e.g., Gaucher's disease, Parkinson's disease). Also provided are pharmaceutical compositions and kits comprising the compounds, and methods of treating GCase-related diseases and disorders (e.g., Gaucher's disease, Parkinson's disease) with the compounds in a subject, by administering the compounds and/or compositions described herein.

### RELATED APPLICATION

This application claims priority under 35 U.S.C. § 119(e) to U.S. Provisional Application 63/255,272, filed Oct. 13, 2021, the entirety of which is incorporated herein by reference.

### BACKGROUND

Glucocerebrosidase (EC 3.2.1.45), also called β-glucocerebrosidase, β-glucosidase, D-glucosyl-N-acylsphingosine glucohydrolase, or GCase, is an enzyme having glucosylceramidase activity. Glucocerebrosidase is required to cleave the beta-glucosidic linkage of the chemical glucocerebroside, which is an intermediate in glycolipid metabolism. Glucocerebrosidase is localized in the lysosome and disabling mutations in the gene for glucocerebrosidase (GBA1) are associated with abnormal accumulation of lipids in lysosomes.

Genetic diseases caused by mutations in GBA1 include neurodegenerative diseases such as Gaucher's disease and Parkinson's disease. Current treatments for diseases such Type 1 Gaucher's disease are limited to enzyme replacement therapy (ERT) administered every two weeks. ERT is very expensive and not effective for neuronopathic forms of Gaucher's disease. Efforts to discover and employ small molecule compounds to activate Gcase have been met with limited success. Thus, there is a need for new compounds that effectively activate Gcase and are useful in the treatment of neurodegenerative diseases (e.g., Gaucher's disease and Parkinson's disease).

### SUMMARY

The present disclosure provides compounds that are modulators of GCase. These compounds provide new compositions and methods for the treatment of diseases associated with GCase activity (e.g., neurodegenerative diseases, such as Gaucher's disease and Parkinson's disease).

In one aspect, provided are compounds of Formula (I):

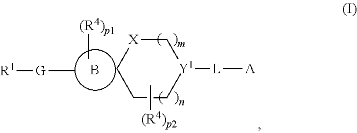

and pharmaceutically acceptable salts, co-crystals, tautomers, stereoisomers, solvates, hydrates, polymorphs, isotopically enriched derivatives, or prodrugs thereof, wherein:
R¹ is substituted or unsubstituted heteroaryl, substituted or unsubstituted aryl, haloalkyl, or a nitrogen protecting group;
G is a bond, —NR^{A}—, —C(═O)—, —SO₂—, —O—, —OC(R⁵)(R⁶)—, or —C(R⁵)(R⁶)—;
B is cycloalkyl or heterocyclyl comprising at least one nitrogen atom in its ring;
each occurrence of R⁴ is independently halogen, substituted or unsubstituted alkyl, substituted or unsubstituted alkoxy, substituted or unsubstituted cycloalkyl, substituted or unsubstituted aryl, or two instances of R⁴ on different carbons combine to form a bridged ring, or two instances of R⁴ on the same carbon form with that carbon a carbonyl or a cycloalkyl;
X is —O—, —NR^{A}—, or —C(R⁴)₂—;
Y¹ is N or CR⁵;
each occurrence of R⁵ and R⁶ are each independently hydrogen, halogen, or substituted or unsubstituted alkyl;
L is a bond, —SO₂—, —C(═O)—, —C(═O)C(R⁵)(R⁶)—, —NR^{A}, or —NHC(═O)—;
A is

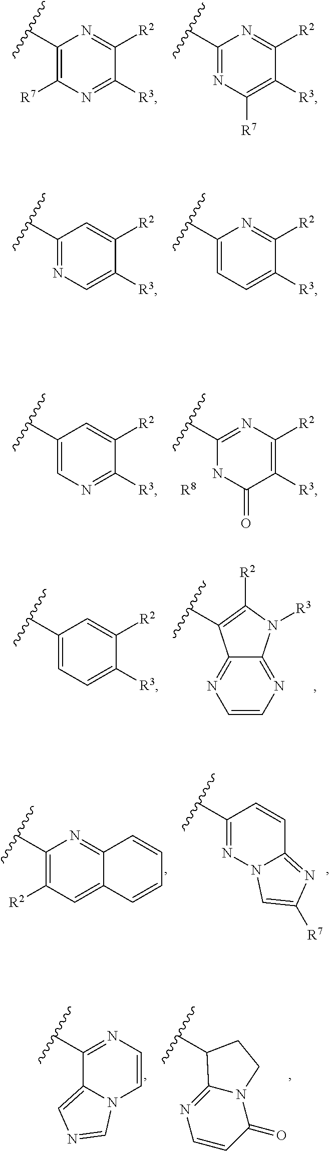

or substituted or unsubstituted 5-membered heteroaryl; R² and R³ are each independently hydrogen, halogen, substituted or unsubstituted alkyl, substituted or unsubstituted alkoxy, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
R⁷ is hydrogen, substituted or unsubstituted alkyl, or substituted or unsubstituted alkoxy;
R⁸ is substituted or unsubstituted alkyl;
each R^{A} is independently hydrogen, substituted or unsubstituted alkyl, or a nitrogen protecting group;
m and n are each independently 0, 1, or 2; and
p1 and p2 are each independently 0, 1, 2, 3, 4, 5, or 6.

In certain embodiments, the compounds of Formula (I) are compounds of Formula (I-l), (I-a), (I-b), (I-g), (I-g-13), (I-k), (I-k-3), (I-k-5), (I-l), (I-l-3), (I-l-5), (I-m), (I-m-3), (I-n), (I-n′), (I-o), (I-o′), (I-p), (I-p′), (I-x), (I-x-1), (I-y), (I-z), (I-aa), (I-bb), (I-cc), (I-dd), (I-ee), (I-ff), or (I-gg):

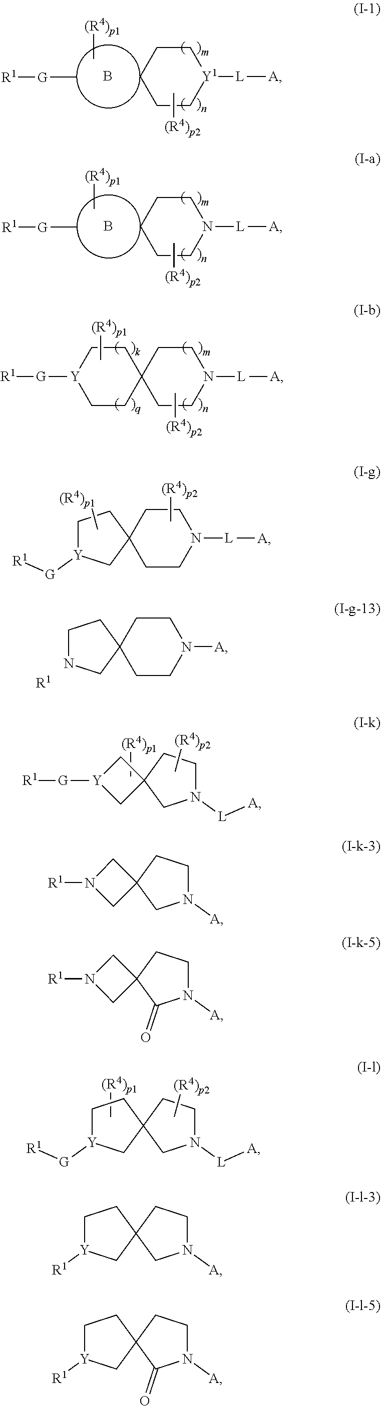

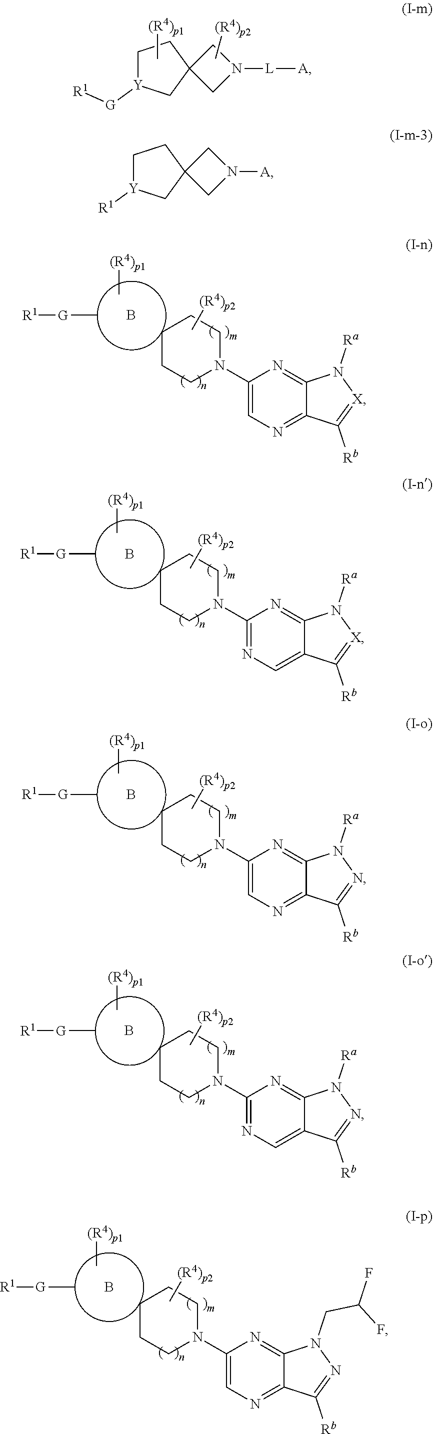

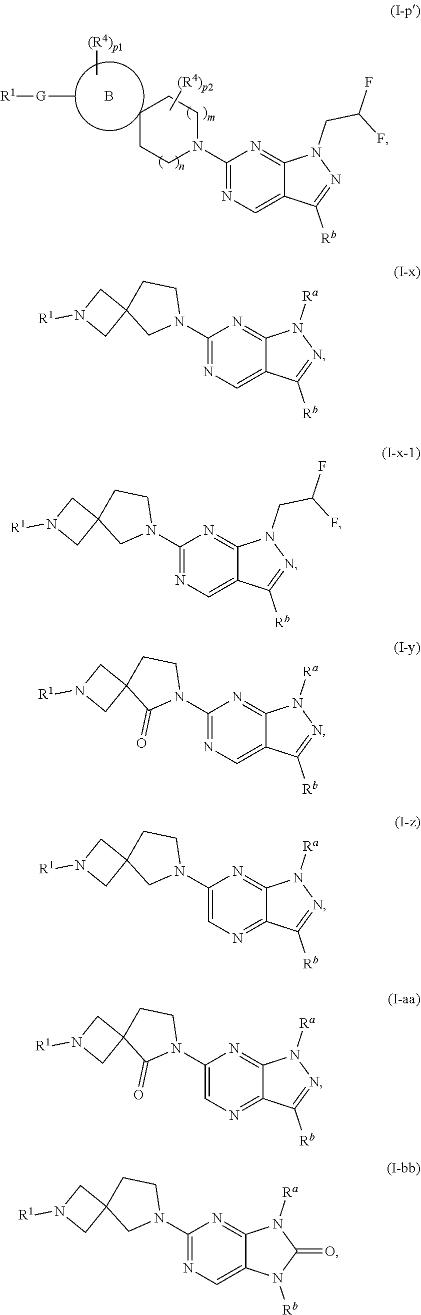

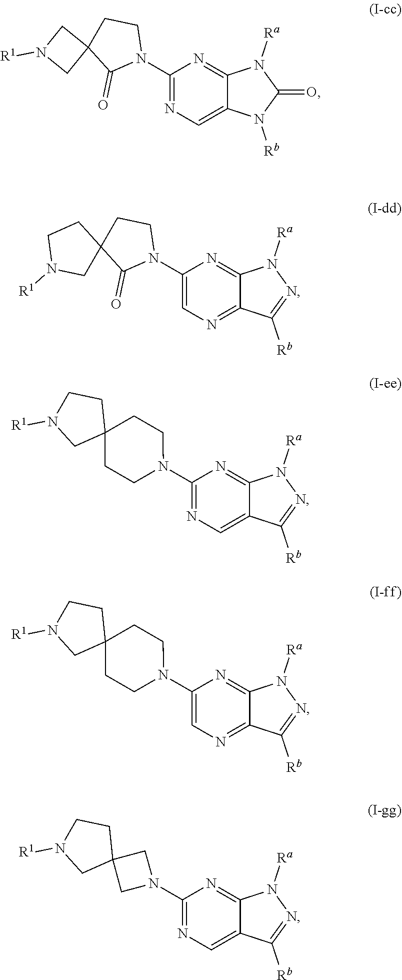

or pharmaceutically acceptable salts thereof.

In another aspect, provided are pharmaceutical compositions comprising a compound of Formula (I), or a pharmaceutically acceptable salt thereof, and optionally a pharmaceutically acceptable excipient.

In another aspect, provided are methods of treating a disease or disorder in a subject in need thereof, the method comprising administering a compound of Formula (I), or a pharmaceutically acceptable salt thereof, or a pharmaceutical composition comprising a compound of Formula (I) to the subject.

In certain embodiments, the disease or disorder is associated with glucocerebrosidase activity. In certain embodiments, the disease or disorder is a neurological disease or disorder. In certain embodiments, the neurological disease or disorder is Parkinson's disease or Gaucher's disease.

In another aspect, provided are methods of activating glucocerebrosidase, the method comprising contacting glucocerebrosidase with an effective amount of a compound of Formula (I), or a pharmaceutically acceptable salt thereof, or a pharmaceutical composition comprising a compound of Formula (I) to the subject.

In another aspect, provided are kits comprising a compound of Formula (I), or a pharmaceutically acceptable salt thereof, or a pharmaceutical composition comprising a compound of Formula (I), or a pharmaceutically acceptable salt thereof. In certain embodiments, the kits further comprise instructions for administration (e.g., human administration).

The details of certain embodiments of the invention are set forth in the Detailed Description of Certain Embodiments, as described below. Other features, objects, and advantages of the invention will be apparent from the Definitions, Examples, and Claims.

### Definitions

### Chemical definitions

Definitions of specific functional groups and chemical terms are described in more detail below. The chemical elements are identified in accordance with the Periodic Table of the Elements, CAS version, Handbook of Chemistry and Physics, 75^{t} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. While compounds may be depicted as racemic or as one or more diastereoisomers, enantiomers, or other isomers, all such racemic, diastereoisomer, enantiomer, or other isomer forms of that depicted are included in the present disclosure.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or 14C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-6 alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

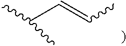

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂-6 alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Arylalkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroarylalkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

When substituted, exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)Rₐₐ, —CO₂H, —CHO, —C(OR^{aa})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, =NR^{bb}, or =NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl) —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{cc})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula: —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and Rad are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R′)₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), 2-methoxyethyl, methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{a}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R)₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄⁻, B(C₆F₅)₄, BPh₄, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(=NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

These and other exemplary substituents are described in more detail in the Detailed Description, Examples, and Claims. The invention is not intended to be limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and/or animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water molecules. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting points, density, hardness, crystal shape, optical and electrical properties, stability, solubility, and bioavailability. Recrystallization solvent, rate of crystallization, storage temperature, and other factors may cause one crystal form to dominate a given preparation. Various polymorphs of a compound can be prepared by crystallization under different conditions.

The term “co-crystal” refers to a crystalline structure composed of at least two components. In certain embodiments, a co-crystal contains a compound of the present disclosure and one or more other component(s), including, but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including, but not limited to, an isomer, tautomer, salt, solvate, hydrate, synthetic precursor, synthetic derivative, fragment, or impurity of said compound.

The term “prodrugs” refers to compounds that have cleavable groups that are removed, by solvolysis or under physiological conditions, to provide the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

The term “modulate” means decreasing or inhibiting activity and/or increasing or augmenting activity. For example, modulating glucocerebrosidase activity means decreasing or inhibiting glucocerebrosidase activity and/or increasing or augmenting glucocerebrosidase activity. The compounds disclosed herein may be administered to modulate glucocerebrosidase activity for example, as a chaperone or activator.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, or inhibiting the progress of a disease described herein. In some embodiments, treatment may be administered after one or more signs or symptoms of the disease have developed or have been observed. Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for GCase activation (e.g., at least 5%, at least 10%, at least 20%, at least 30%, at least 40%, at least 50%, at least 60%, at least 70%, at least 80%, at least 90%, at least 95%, at least 100%, at least 150%, at least 200%, at least 250%, at least 300%, or at least 500% increase in the enzymatic activity of GCase). In certain embodiments, a therapeutically effective amount is an amount sufficient for treating a disease or disorder (e.g., neurological disorder). In certain embodiments, a therapeutically effective amount is an amount sufficient for GCase activation and treating a disease or disorder (e.g., neurological disorder).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for GCase activation. In certain embodiments, a prophylactically effective amount is an amount sufficient for treating a disease or disorder (e.g., neurological disorder). In certain embodiments, a prophylactically effective amount is an amount sufficient for GCase activation and treating a disease or disorder (e.g., neurological disorder).

As used herein, the term “activate” or “activation” in the context of enzymes, for example, in the context of GCase, refers to an increase in the activity of the enzyme. In some embodiments, the term refers to an increase of the level of enzyme activity, e.g., GCase activity, to a level that is statistically significantly higher than an initial level, which may, for example, be a baseline level of enzyme activity (e.g., of wild-type GCase). In some embodiments, the term refers to an increase in the level of enzyme activity, e.g., GCase activity, to a level that is greater than 1%, greater than 5%, greater than 10%, greater than 25%, greater than 50%, greater than 75%, greater than 100%, greater than 150%, greater than 200%, greater than 300%, greater than 400%, greater than 500%, or greater than 1000% of an initial level, which may, for example, be a baseline level of enzyme activity.

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics, or combinations thereof.

### DETAILED DESCRIPTION OF CERTAIN EMBODIMENTS

Provided herein are compounds that are modulators of GCase (e.g., GCase activators). In one aspect, the provided GCase modulators are compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. Accordingly, the compounds are useful for the treatment and/or prevention of diseases and disorders associated with GCase activity (e.g., neurological diseases and disorders) in a subject in need thereof.

The compounds described herein interact with GCase. As described herein, the therapeutic effect may be a result of modulation (e.g., activation), binding, and/or modification of GCase by the compounds described herein. The compounds may be provided for use in any composition, kit, or method described herein as a pharmaceutically acceptable salt, co-crystal, tautomer, stereoisomer, solvate, hydrate, polymorph, isotopically enriched derivative, or prodrug thereof.

### Compounds of Formula (I)

In one aspect, disclosed is a compound of Formula (I):

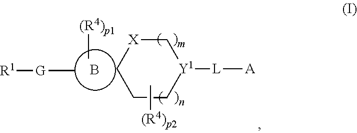

or a pharmaceutically acceptable salt, co-crystal, tautomer, stereoisomer, solvate, hydrate, polymorph, isotopically enriched derivative, or prodrug thereof, wherein:
R¹ is substituted or unsubstituted heteroaryl, substituted or unsubstituted aryl, haloalkyl, or a nitrogen protecting group;
G is a bond, —NR^{A}—, —C(═O)—, —SO₂—, —O—, —OC(R⁵)(R⁶)—, or —C(R⁵)(R⁶)—;
B is cycloalkyl or heterocyclyl comprising at least one nitrogen atom in its ring;
each occurrence of R⁴ is independently halogen, substituted or unsubstituted alkyl, substituted or unsubstituted alkoxy, substituted or unsubstituted cycloalkyl, substituted or unsubstituted aryl, or two instances of R⁴ on different carbons combine to form a bridged ring, or two instances of R⁴ on the same carbon form with that carbon a carbonyl or a cycloalkyl;
X is —O—, —NR^{A}—, or —C(R⁴)₂—;
Y¹ is N or CR⁵;
each occurrence of R⁵ and R⁶ are each independently hydrogen, halogen, or substituted or unsubstituted alkyl;
L is a bond, —SO₂—, —C(═O)—, —C(═O)C(R⁵)(R⁶)—, —NR^{A}—, or —NHC(═O)—;
A is

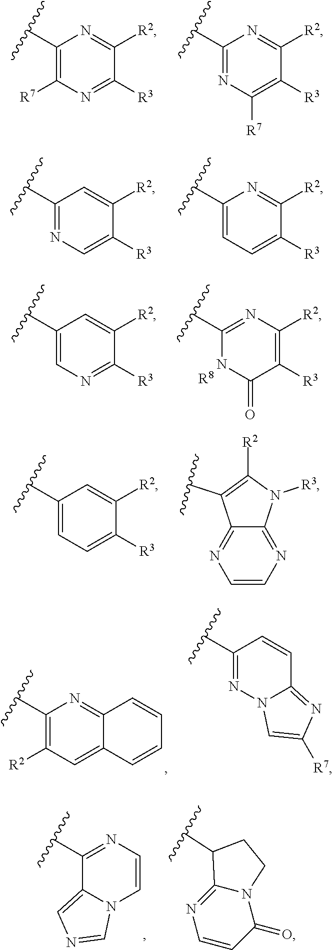

or substituted or unsubstituted 5-membered heteroaryl;
R² and R³ are each independently hydrogen, halogen, substituted or unsubstituted alkyl, substituted or unsubstituted alkoxy, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
R⁷ is hydrogen, substituted or unsubstituted alkyl, or substituted or unsubstituted alkoxy;
R⁸ is substituted or unsubstituted alkyl;
each R^{A} is independently hydrogen, substituted or unsubstituted alkyl, or a nitrogen protecting group;
m and n are each independently 0, 1, or 2; and
p1 and p2 are each independently 0, 1, 2, 3, 4, 5, or 6.

In certain embodiments, the compound of Formula (I) is a compound of Formula (I-1):

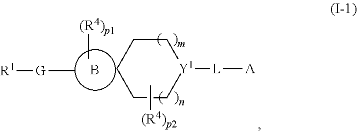

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of Formula (I-1):

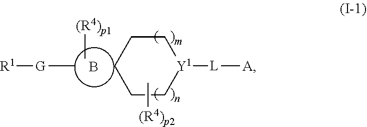

or a pharmaceutically acceptable salt, co-crystal, tautomer, stereoisomer, solvate, hydrate, polymorph, isotopically enriched derivative, or prodrug thereof, wherein:
R¹ is substituted or unsubstituted heteroaryl, substituted or unsubstituted aryl, or a nitrogen protecting group;
G is a bond, —O—, —OC(R⁵)(R⁶)—, or —C(R⁵)(R⁶)—;
B is cycloalkyl or heterocyclyl comprising at least one nitrogen atom in its ring;
each occurrence of R⁴ is independently halogen, substituted or unsubstituted alkyl, substituted or unsubstituted alkoxy, or two instances of R⁴ on the same carbon form with that carbon a carbonyl;
Y¹ is N or CR⁵;
each occurrence of R⁵ and R⁶ are each independently hydrogen, halogen, or substituted or unsubstituted alkyl;
L is a bond, —SO₂—, —C(═O)—, —C(═O)C(R⁵)(R⁶)—, or —NHC(═O)—;
A is

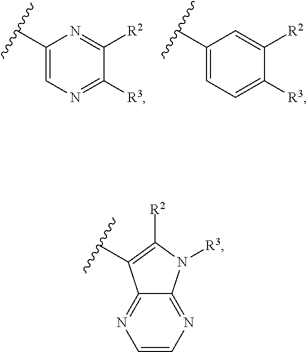

or substituted or unsubstituted 5-membered heteroaryl;
R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
m and n are each independently 0, 1, or 2; and
p1 and p2 are each independently 0, 1, 2, 3, 4, 5, or 6.

### R¹

As described herein, R¹ is substituted or unsubstituted heteroaryl, substituted or unsubstituted aryl, haloalkyl, or a nitrogen protecting group. In certain embodiments, R¹ is substituted or unsubstituted heteroaryl, substituted or unsubstituted aryl, or a nitrogen protecting group. In certain embodiments, R¹ is substituted or unsubstituted heteroaryl, or substituted or unsubstituted aryl. In certain embodiments, R¹ is substituted or unsubstituted heteroaryl, or a nitrogen protecting group.

In certain embodiments, RV is a nitrogen protecting group. In certain embodiments, R¹ is 2-methoxyethyl. In certain embodiments, R¹ is a carbamate. In certain embodiments, R¹ is —C(═O)OR^{aa}, wherein R^{aa} is as defined herein. In certain embodiments, R¹ is t-butyl carbamate (i.e., BOC).

In certain embodiments, R¹ is haloalkyl. In certain embodiments, R¹ is —CH₂CH₂CF₃.

In certain embodiments, R¹ is substituted or unsubstituted heteroaryl. In certain embodiments, R¹ is substituted or unsubstituted pyridinyl, substituted or unsubstituted pyrazinyl, substituted or unsubstituted pyrimidinyl, substituted or unsubstituted pyridazinyl, substituted or unsubstituted thiazolyl, substituted or unsubstituted oxadiazolyl, substituted or unsubstituted thiadiazolyl, substituted or unsubstituted isoxozalyl, substituted or unsubstituted oxazolyl, substituted or unsubstituted thiazolyl, substituted or unsubstituted pyridinonyl, or substituted or unsubstituted pyridazinonyl.

In certain embodiments, R¹ is substituted or unsubstituted 6-membered heteroaryl. In certain embodiments, R¹ is substituted or unsubstituted pyridinyl, substituted or unsubstituted pyrazinyl, substituted or unsubstituted pyrimidinyl, or substituted or unsubstituted pyridazinyl. In certain embodiments, R¹ is substituted or unsubstituted pyridinyl. In certain embodiments, R¹ is substituted or unsubstituted pyrazinyl. In certain embodiments, R¹ is substituted or unsubstituted pyrimidinyl. In certain embodiments, R¹ is substituted or unsubstituted pyridazinyl.

In certain embodiments, R¹ is heteroaryl substituted with at least one instance of halogen, haloalkyl or haloalkoxy. In certain embodiments, R¹ is heteroaryl substituted with at least one instance of halogen or haloalkyl. In certain embodiments, R¹ is heteroaryl substituted with at least one instance of halogen, C₁₋₄ haloalkyl or C₁₋₄ haloalkoxy. In certain embodiments, R¹ is heteroaryl substituted with at least one instance of halogen or C₁₋₄ haloalkyl. In certain embodiments, R¹ is heteroaryl substituted with at least one instance of fluoro or fluoroalkyl. In certain embodiments, R¹ is heteroaryl substituted with at least one instance of fluoro or C₁₋₄ fluoroalkyl. In certain embodiments, R¹ is heteroaryl substituted with at least one instance of C₁₋₄ fluoroalkyl.

In certain embodiments, R¹ is substituted or unsubstituted pyridinyl. In certain embodiments, R¹ is substituted pyridinyl. In certain embodiments, R¹ is pyridinyl substituted with at least one instance of halogen, haloalkyl or haloalkoxy. In certain embodiments, R¹ is pyridinyl substituted with at least one instance of halogen or haloalkyl. In certain embodiments, R¹ is pyridinyl substituted with at least one instance of halogen or C₁₋₄ haloalkyl or C₁₋₄ haloalkoxy. In certain embodiments, R¹ is pyridinyl substituted with at least one instance of halogen or C₁₋₄ haloalkyl. In certain embodiments, R¹ is pyridinyl substituted with at least one instance of fluoro or fluoroalkyl. In certain embodiments, R¹ is pyridinyl substituted with at least one instance of fluoro or C₁₋₄ fluoroalkyl.

In certain embodiments, R¹ is heteroaryl substituted with at least one instance of haloalkyl. In certain embodiments, R¹ is heteroaryl substituted with at least one instance of C₁₋₄ haloalkyl. In certain embodiments, R¹ is heteroaryl substituted with at least one instance of fluoroalkyl. In certain embodiments, R¹ is heteroaryl substituted with at least one instance of C₁₋₄ fluoroalkyl. In certain embodiments, R¹ is heteroaryl substituted with at least one instance of trifluoromethyl, difluoromethyl, or fluoromethyl. In certain embodiments, R¹ is heteroaryl substituted with trifluoromethyl.

In certain embodiments, R¹ is pyridinyl substituted with at least one instance of haloalkyl. In certain embodiments, R¹ is pyridinyl substituted with at least one instance of C₁₋₄ haloalkyl. In certain embodiments, R¹ is pyridinyl substituted with at least one instance of fluoroalkyl. In certain embodiments, R¹ is pyridinyl substituted with at least one instance of C₁₋₄ fluoroalkyl. In certain embodiments, R¹ is pyridinyl substituted with at least one instance of trifluoromethyl, difluoromethyl, or fluoromethyl. In certain embodiments, R¹ is pyridinyl substituted with at least one instance of trifluoromethyl.

In certain embodiments, R¹ is pyrimidinyl substituted with at least one instance of haloalkyl. In certain embodiments, R¹ is pyrimidinyl substituted with at least one instance of C₁₋₄ haloalkyl. In certain embodiments, R¹ is pyrimidinyl substituted with at least one instance of fluoroalkyl. In certain embodiments, R¹ is pyrimidinyl substituted with at least one instance of C₁₋₄ fluoroalkyl. In certain embodiments, R¹ is pyrimidinyl substituted with at least one instance of trifluoromethyl, difluoromethyl, or fluoromethyl. In certain embodiments, R¹ is pyrimidinyl substituted with at least one instance of trifluoromethyl.

In certain embodiments, R¹ is substituted or unsubstituted pyrimidinyl. In certain embodiments, R¹ is substituted pyrimidinyl. In certain embodiments, R¹ is pyrimidinyl substituted with at least one instance of halogen, unsubstituted alkyl, haloalkyl or haloalkoxy. In certain embodiments, R¹ is pyrimidinyl substituted with at least one instance of unsubstituted alkyl or haloalkyl. In certain embodiments, R¹ is pyrimidinyl substituted with at least one instance of unsubstituted C₁₋₄ alkyl or C₁₋₄ haloalkyl. In certain embodiments, R¹ is pyrimidinyl substituted with at least one instance of unsubstituted C₁₋₄ alkyl or C₁₋₄ fluoroalkyl. In certain embodiments, R¹ is pyrimidinyl substituted with at least one instance of methyl or C₁₋₄ fluoroalkyl.

In certain embodiments, R¹ is pyrimidinyl substituted with unsubstituted alkyl and haloalkyl. In certain embodiments, R¹ is pyrimidinyl substituted with unsubstituted C₁₋₄ alkyl and C₁₋₄ haloalkyl. In certain embodiments, R¹ is pyrimidinyl substituted with methyl and fluoroalkyl. In certain embodiments, R¹ is pyrimidinyl substituted with methyl and C₁₋₄ fluoroalkyl. In certain embodiments, R¹ is pyrimidinyl substituted with methyl and trifluoromethyl, difluoromethyl, or fluoromethyl. In certain embodiments, R¹ is pyrimidinyl substituted with methyl and trifluoromethyl.

In certain embodiments, R¹ is substituted or unsubstituted aryl. In certain embodiments, R¹ is substituted or unsubstituted phenyl. In certain embodiments, R¹ is unsubstituted phenyl. In certain embodiments, R¹ is unsubstituted phenyl or phenyl substituted with at least one instance of halogen or haloalkyl. In certain embodiments, R¹ is phenyl substituted with at least one instance of C₁₋₄ haloalkyl or halogen. In certain embodiments, R¹ is phenyl substituted with at least one instance of fluoroalkyl or halogen. In certain embodiments, R¹ is phenyl substituted with at least one instance of C₁₋₄ fluoroalkyl or halogen. In certain embodiments, R¹ is phenyl substituted with at least one instance of C₁₋₄ fluoroalkyl or fluoro.

In certain embodiments, R¹ is phenyl substituted with at least one instance of haloalkyl or alkyl. In certain embodiments, R¹ is phenyl substituted with at least one instance of C₁₋₄ haloalkyl or C₁₋₄ alkyl. In certain embodiments, R¹ is phenyl substituted with at least one instance of fluoroalkyl or alkyl. In certain embodiments, R¹ is phenyl substituted with at least one instance of C₁₋₄ fluoroalkyl or C₁₋₄ alkyl.

In certain embodiments, R¹ is phenyl substituted with at least one instance of fluoroalkyl. In certain embodiments, R¹ is phenyl substituted with at least one instance of C₁₋₄ fluoroalkyl. In certain embodiments, R¹ is phenyl substituted with at least one instance of fluoroalkyl. In certain embodiments, R¹ is phenyl substituted with at least one instance of C₁₋₄ fluoroalkyl.

In certain embodiments, R¹ is phenyl substituted with at least one instance of alkyl. In certain embodiments, R¹ is phenyl substituted with at least one instance of C₁₋₄ alkyl. In certain embodiments, R¹ is phenyl substituted with at least one instance of halogen. In certain embodiments, R¹ is phenyl substituted with at least one instance of fluoro.

In certain embodiments, R¹ is

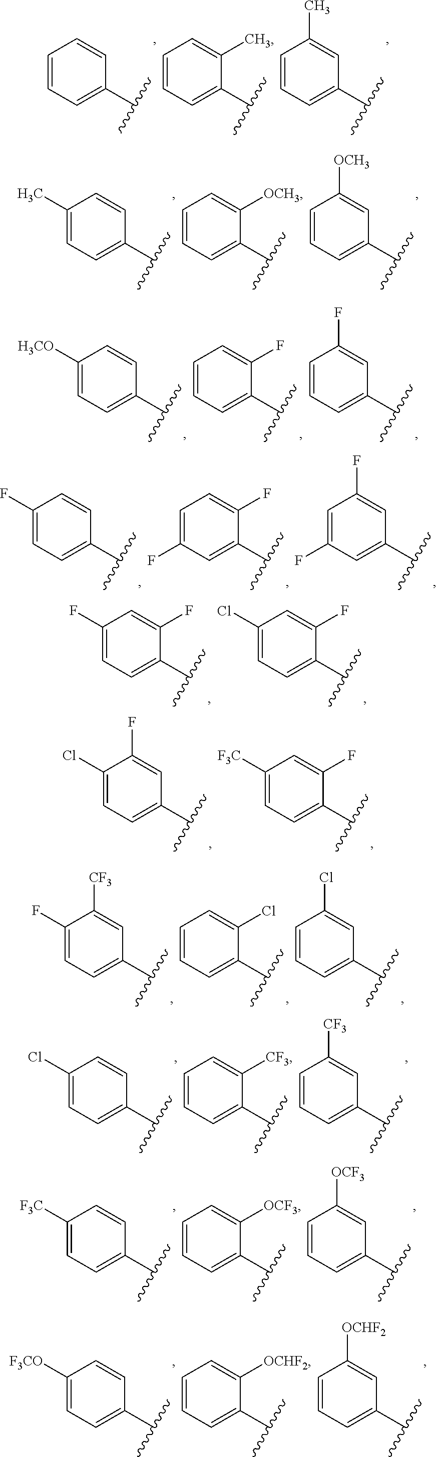

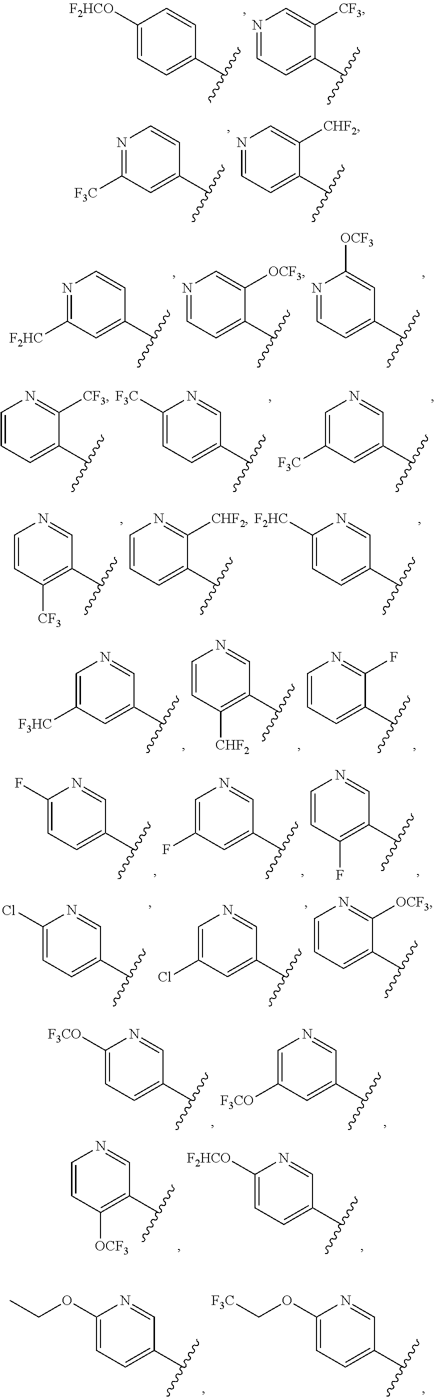

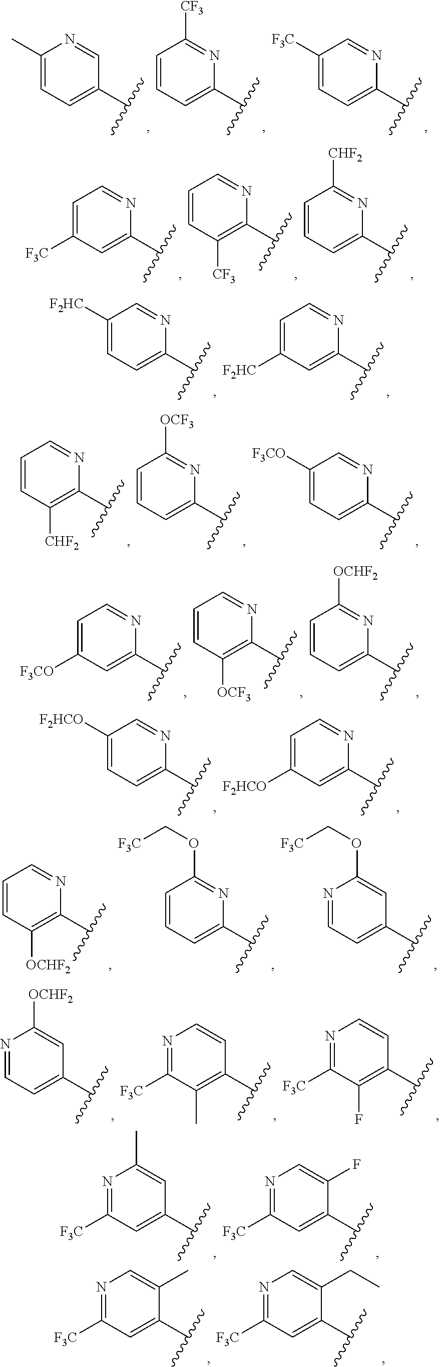

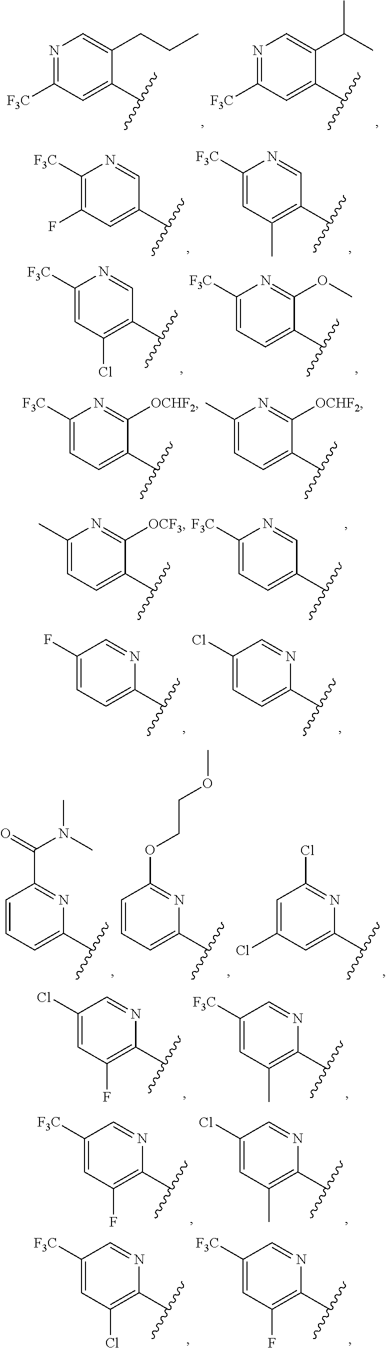

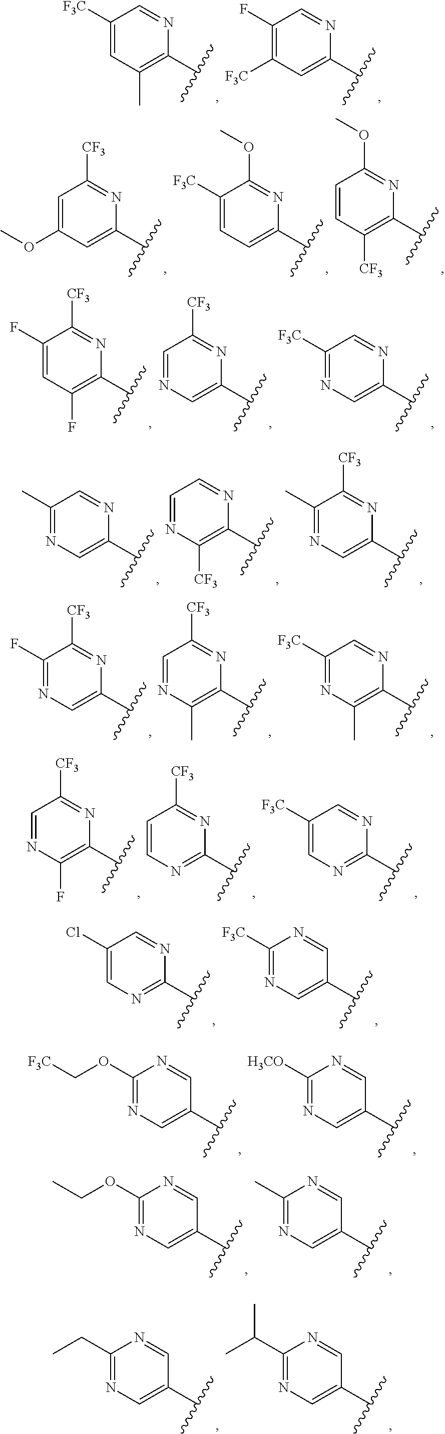

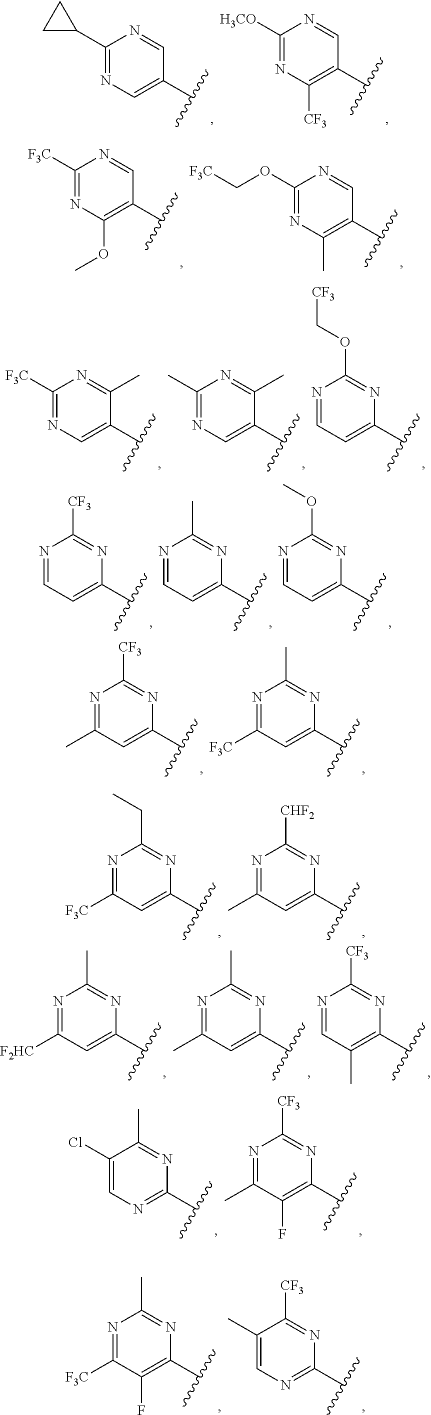

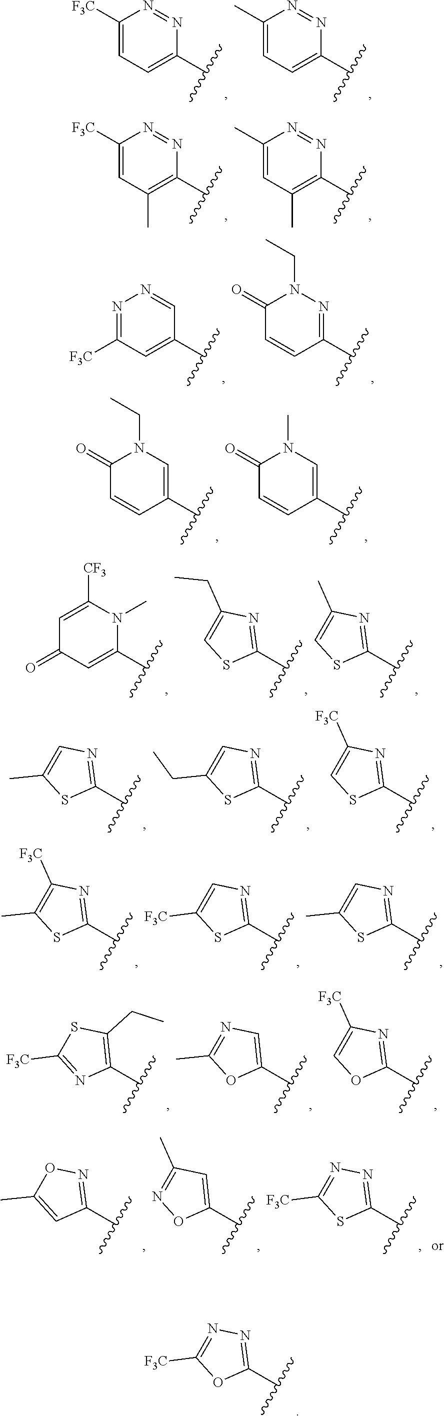

In certain embodiments, R¹ is

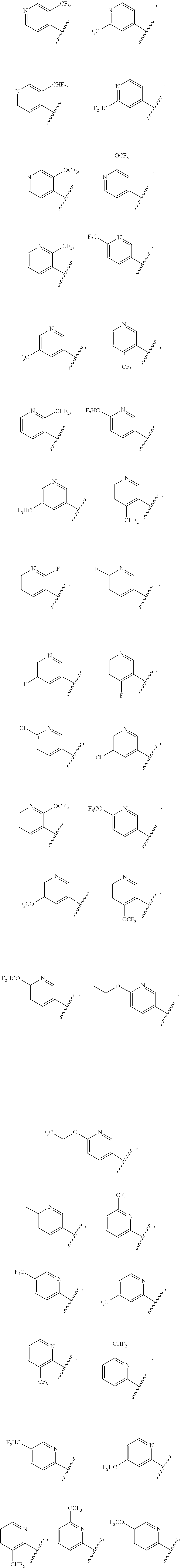

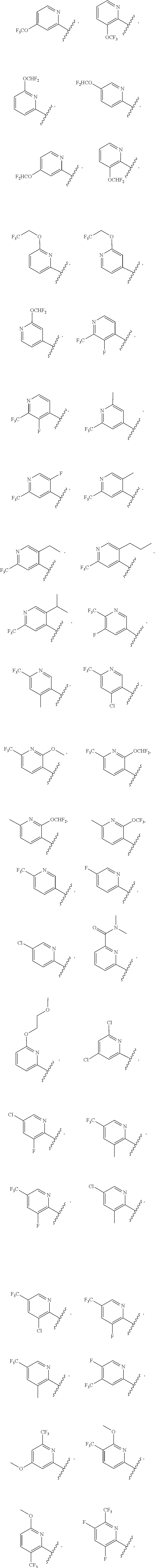

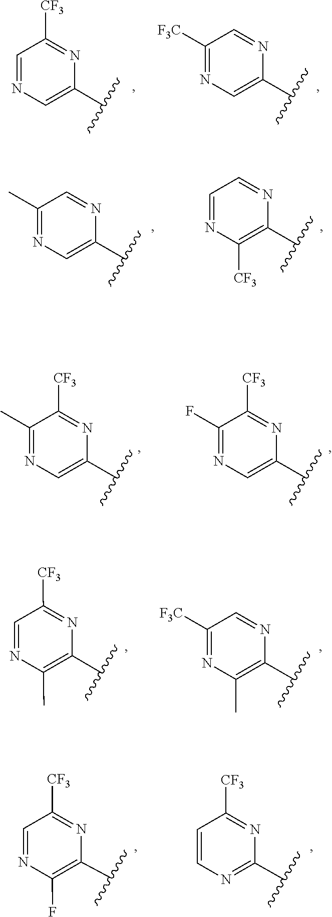

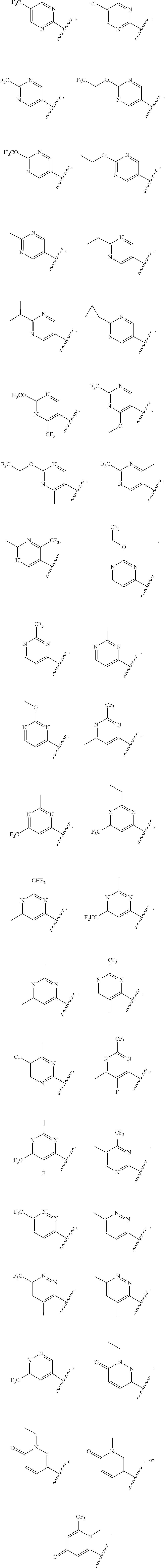

In certain embodiments, R¹ is

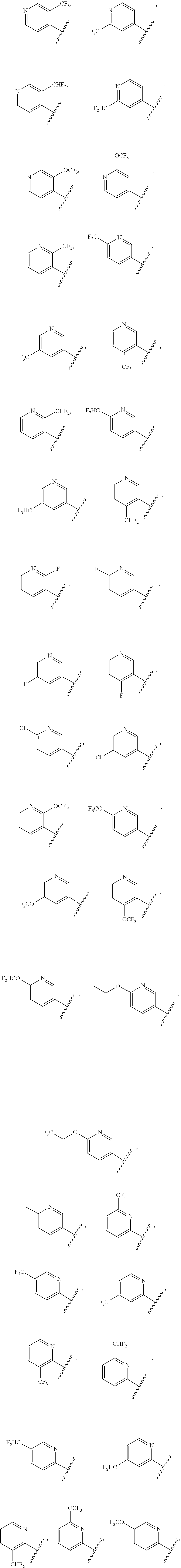

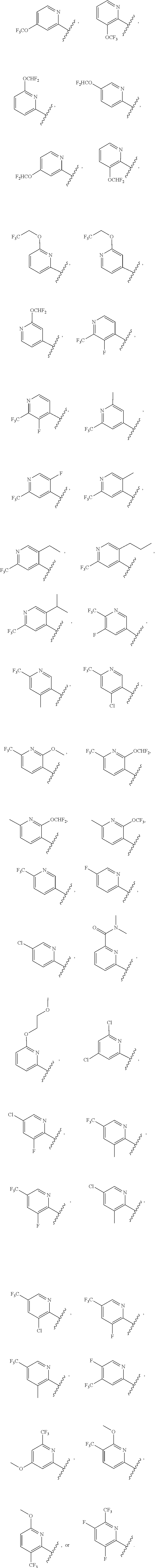

In certain embodiments, R¹ is

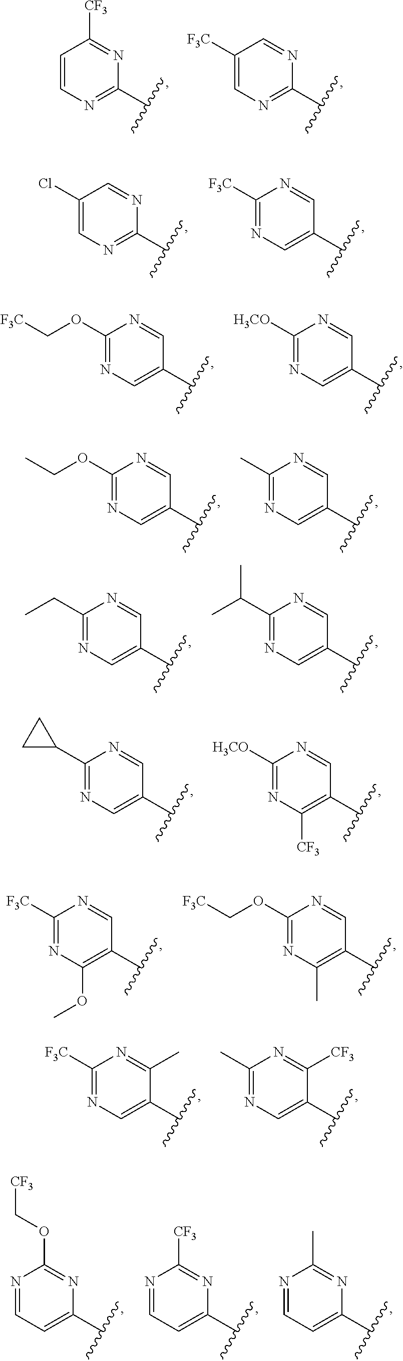

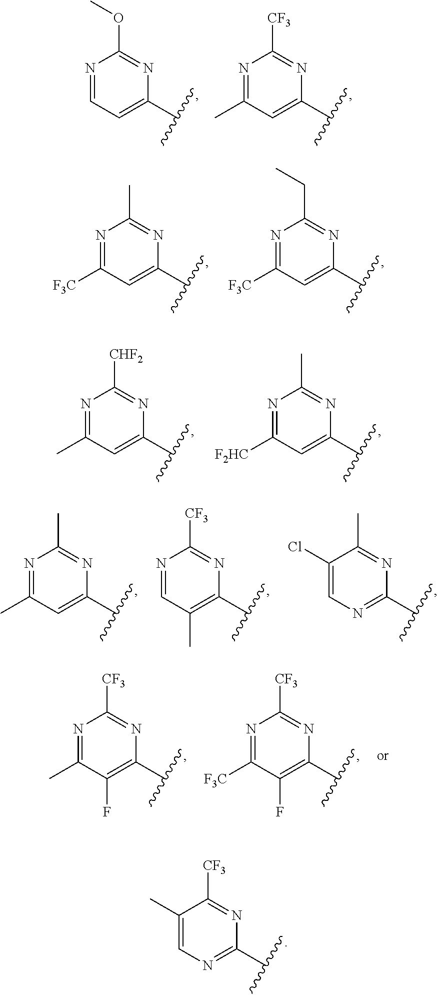

In certain embodiments, R¹ is

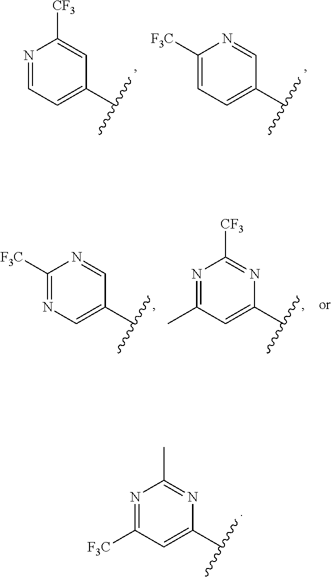

In certain embodiments, R¹ is

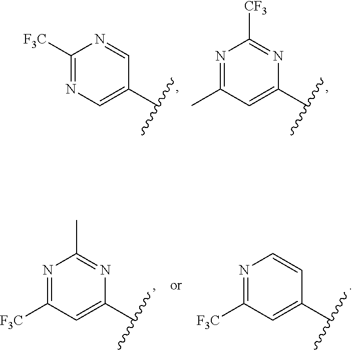

In certain embodiments, R¹

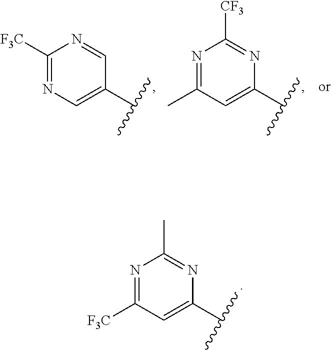

In certain embodiments, R¹ is

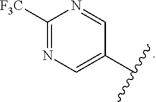 [SMILES: *c1cnc(C(F)(F)F)nc1]

In certain embodiments, R¹ is

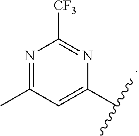 [SMILES: *c1cc(C)nc(C(F)(F)F)n1]

In certain embodiments, R¹ is

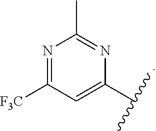 [SMILES: *c1cc(C(F)(F)F)nc(C)n1]

In certain embodiments, R¹ is

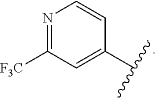 [SMILES: *c1ccnc(C(F)(F)F)c1]

In certain embodiments, R¹ is

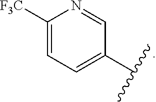 [SMILES: *c1ccc(C(F)(F)F)nc1]

In certain embodiments, R¹ is

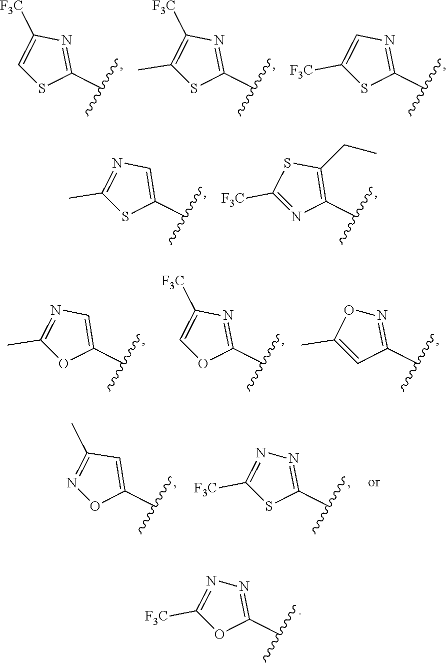

In certain embodiments, R¹ is

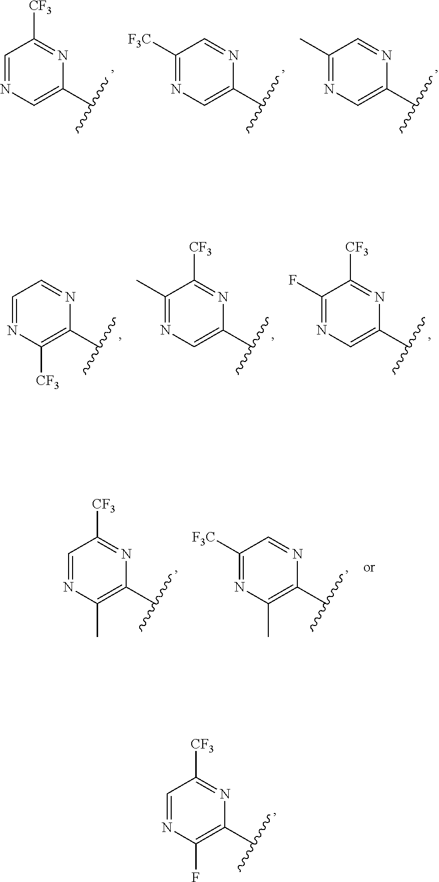

In certain embodiments, R¹ is

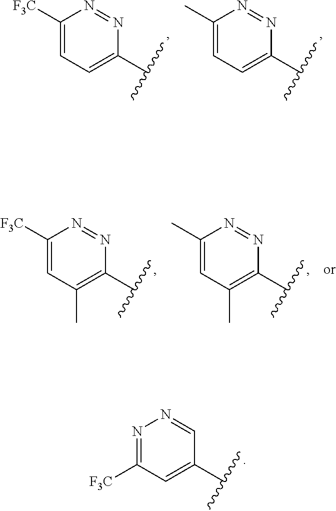

In certain embodiments, R¹ is

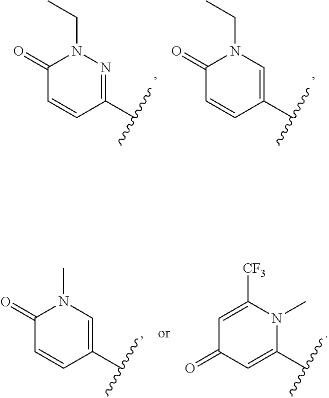

In certain embodiments, R¹ is

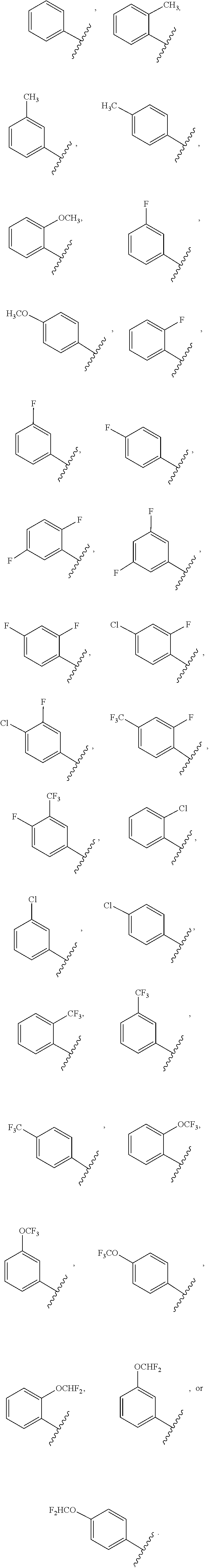

In certain embodiments, R¹ is

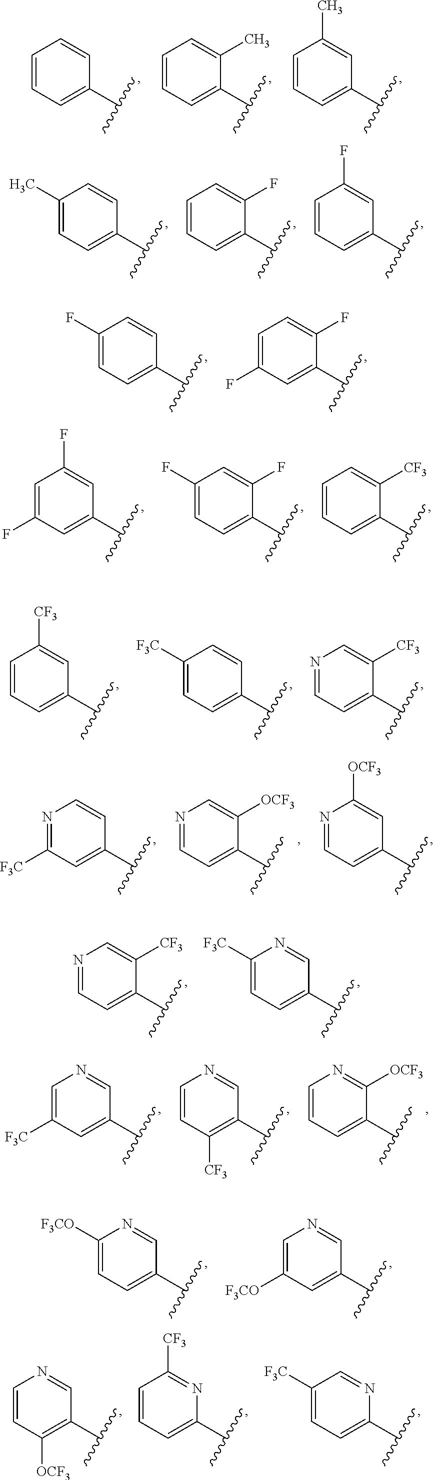

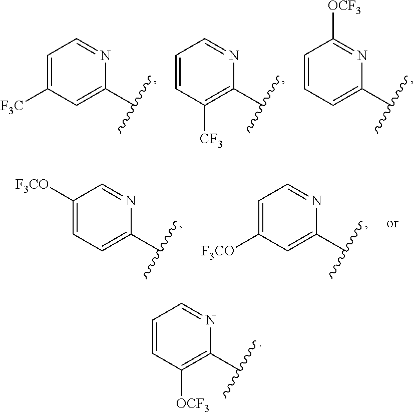

In certain embodiments, R¹ is

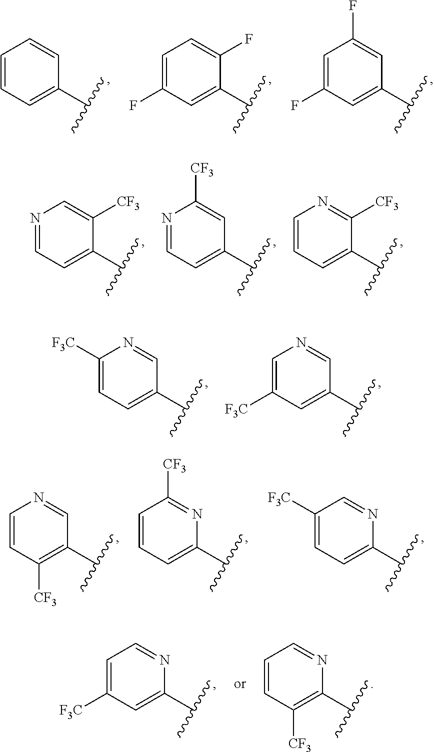

In certain embodiments, R¹ is

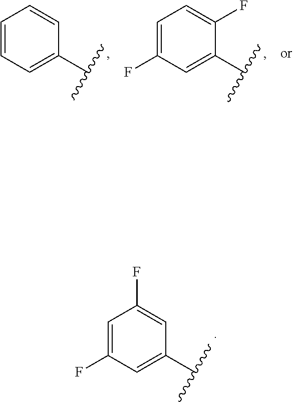

In certain embodiments, R¹ is

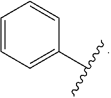 [SMILES: *c1ccccc1]

In certain embodiments, R¹ is

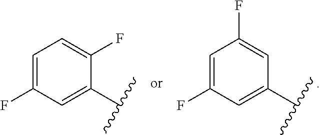 [SMILES: *c1cc(F)ccc1F ; *c1cc(F)cc(F)c1]

In certain embodiments, R¹ is

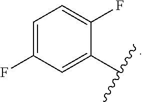 [SMILES: *c1cc(F)ccc1F]

In certain embodiments, R¹ is

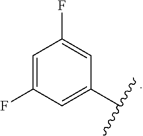 [SMILES: *c1cc(F)cc(F)c1]

In certain embodiments, R¹ is

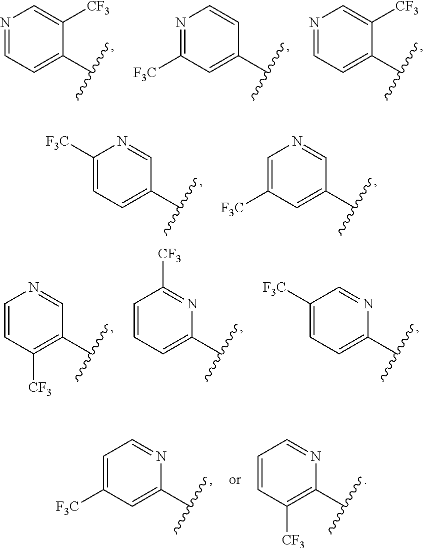

In certain embodiments, R¹ is

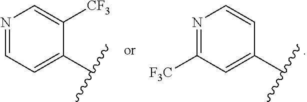 [SMILES: *c1ccncc1C(F)(F)F ; *c1ccnc(C(F)(F)F)c1]

In certain embodiments, R¹ is

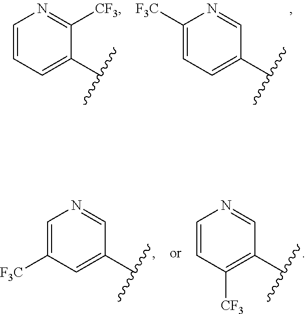 [SMILES: *c1cccnc1C(F)(F)F ; *c1ccc(C(F)(F)F)nc1 ; *c1cncc(C(F)(F)F)c1 ; *c1cnccc1C(F)(F)F]

In certain embodiments, R¹ is

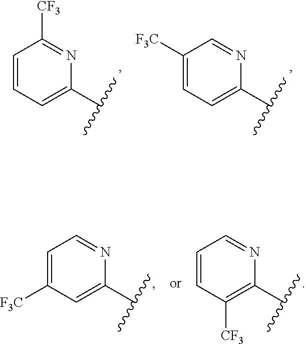 [SMILES: *c1cccc(C(F)(F)F)n1 ; *c1ccc(C(F)(F)F)cn1 ; *c1cc(C(F)(F)F)ccn1 ; *c1ncccc1C(F)(F)F]

In certain embodiments, R¹ is

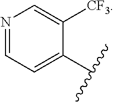 [SMILES: *c1ccncc1C(F)(F)F]

In certain embodiments, R¹ is

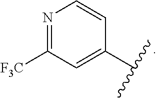 [SMILES: *c1ccnc(C(F)(F)F)c1]

In certain embodiments, R¹ is

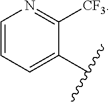 [SMILES: *c1cccnc1C(F)(F)F]

In certain embodiments, R¹ is

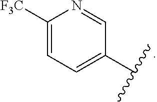 [SMILES: *c1ccc(C(F)(F)F)nc1]

In certain embodiments, R¹ is

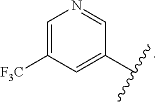 [SMILES: *c1cncc(C(F)(F)F)c1]

In certain embodiments, R¹ is

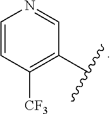 [SMILES: *c1cnccc1C(F)(F)F]

In certain embodiments, R¹ is

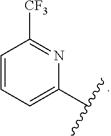 [SMILES: *c1cccc(C(F)(F)F)n1]

In certain embodiments, R¹ is

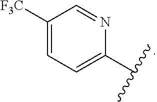 [SMILES: *c1ccc(C(F)(F)F)cn1]

In certain embodiments, R¹ is

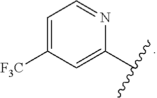 [SMILES: *c1cc(C(F)(F)F)ccn1]

In certain embodiments, R¹ is

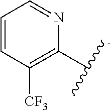 [SMILES: *c1ncccc1C(F)(F)F]

### G

As described herein, G is a bond, —NR^{A}—, —C(═O)—, —SO₂—, —O—, —OC(R⁵)(R⁶)—, or —C(R⁵)(R⁶)—, wherein each occurrence of R⁵ and R⁶ are each independently hydrogen, halogen, or substituted or unsubstituted alkyl. In certain embodiments, G is a bond, —O—, —OC(R⁵)(R⁶)—, or —C(R⁵)(R⁶)—, wherein R⁵ and R⁶ are each independently hydrogen, halogen, or substituted or unsubstituted alkyl. In certain embodiments, R⁵ and R⁶ are each independently hydrogen or substituted or unsubstituted alkyl. In certain embodiments, R⁵ and R⁶ are each independently hydrogen or unsubstituted alkyl. In certain embodiments, R⁵ and R⁶ are each independently hydrogen or unsubstituted C₁₋₄ alkyl. In certain embodiments, R⁵ and R⁶ are each hydrogen.

In certain embodiments, G is a bond, —O—, or —C(R⁵)(R⁶)—. In certain embodiments, G is a bond, —O—, or —OC(R⁵)(R⁶)—. In certain embodiments, G is a bond or —OC(R⁵)(R⁶)—. In certain embodiments, G is a bond or —O—. In certain embodiments, G is a bond. In certain embodiments, G is —O—. In certain embodiments, G is —C(R⁵)(R⁶)—. In certain embodiments, G is —CH₂—. In certain embodiments, G is —OC(R⁵)(R⁶)—. In certain embodiments, G is —OCH₂—. In certain embodiments, G is —NR^{A}—. In certain embodiments, G is —NH—. In certain embodiments, G is —C(═O)—. In certain embodiments, G is —SO₂—.

### B

As described herein, B is cycloalkyl or heterocyclyl comprising at least one nitrogen atom in its ring.

In certain embodiments, B is cycloalkyl. In certain embodiments, B is C₃₋₈ cycloalkyl. In certain embodiments, B is C₃₋₇ cycloalkyl. In certain embodiments, B is C₃₋₆ cycloalkyl. In certain embodiments, B is C₄₋₆ cycloalkyl. In certain embodiments, B is C₄₋₅ cycloalkyl. In certain embodiments, B is C₅₋₆ cycloalkyl. In certain embodiments, B is cyclopropyl, cyclobutyl, cyclopentyl, or cyclohexyl. In certain embodiments, B is cyclobutyl, cyclopentyl, or cyclohexyl. In certain embodiments, B is cyclobutyl or cyclopentyl. In certain embodiments, B is cyclopropyl. In certain embodiments, B is cyclobutyl. In certain embodiments, B is cyclopentyl. In certain embodiments, B is cyclohexyl.

In certain embodiments, B is heterocyclyl comprising at least one nitrogen atom in its ring. In certain embodiments, B is a 3-8 membered heterocyclyl comprising at least one nitrogen atom in its ring. In certain embodiments, B is a 3-8 membered heterocyclyl comprising one nitrogen atom in its ring. In certain embodiments, B is a 3-8 membered heterocyclyl comprising two nitrogen atoms in its ring. In certain embodiments, B is a 3-8 membered heterocyclyl comprising one or two nitrogen atoms in its ring. In certain embodiments, B is azetidinyl, pyrrolidinyl, imidazolidinyl, or piperidinyl. In certain embodiments, B is azetidinyl. In certain embodiments, B is pyrrolidinyl. In certain embodiments, B is imidazolidinyl. In certain embodiments, B is piperidinyl.

In certain embodiments, B is azetidinyl, pyrrolidinyl, imidazolidinyl, piperidinyl, cyclobutyl, cyclopentyl, or cyclohexyl. In certain embodiments, B is azetidinyl, pyrrolidinyl, imidazolidinyl, piperidinyl, cyclobutyl, or cyclopentyl.

In certain embodiments, —B(R⁴)ₚ₁— is

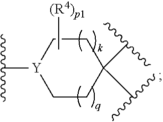

wherein: Y is N or CR⁵; k and q are each independently 0, 1, or 2; and Y is bound to G of Formula (I).

In certain embodiments, Y is N. In certain embodiments, Y is CR⁵. In certain embodiments, Y is CH.

In certain embodiments, k is 0. In certain embodiments, k is 1. In certain embodiments, k is 2. In certain embodiments, q is 0. In certain embodiments, q is 1. In certain embodiments, q is 2. In certain embodiments, k is 0; and q is 0. In certain embodiments, k is 0; and q is 1. In certain embodiments, k is 0; and q is 2. In certain embodiments, k is 1; and q is 0. In certain embodiments, k is 1; and q is 1. In certain embodiments, k is 1; and q is 2. In certain embodiments, k is 2; and q is 0. In certain embodiments, k is 2; and q is 1. In certain embodiments, k is 2; and q is 2.

In certain embodiments, —B(R⁴)ₚ₁— is

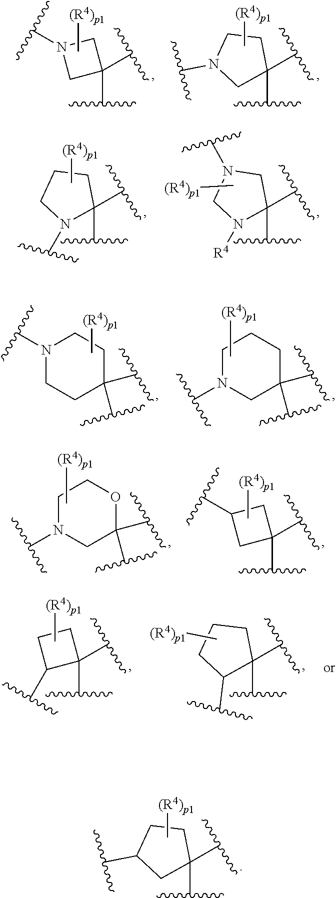

In certain embodiments, —B(R⁴)ₚ₁— is

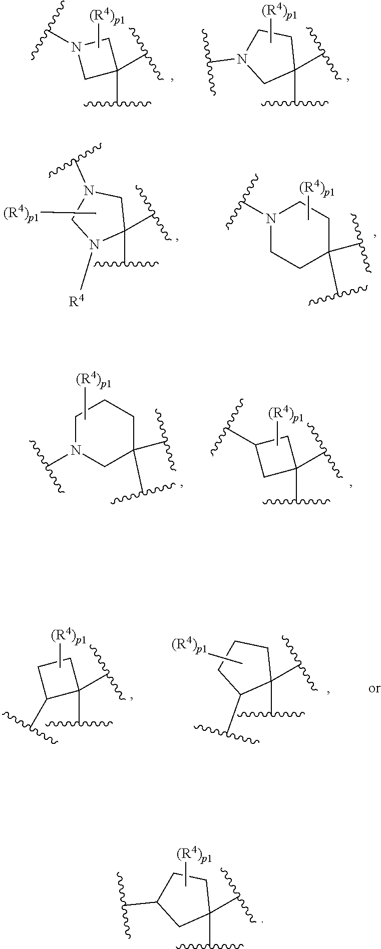

In certain embodiments, —B(R⁴)ₚ₁— is

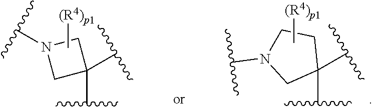

In certain embodiments, —B(R⁴)ₚ₁— is

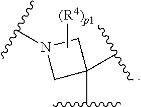

In certain embodiments, —B(R⁴)ₚ₁— is

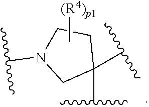

In certain embodiments, —B(R⁴)ₚ₁— is

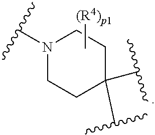

In certain embodiments, —B(R⁴)ₚ₁— is

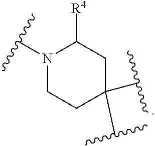

In certain embodiments, —B(R⁴)ₚ₁— is

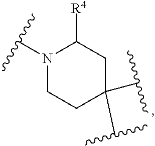

wherein R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl. In certain embodiments, —B(R⁴)ₚ₁— is

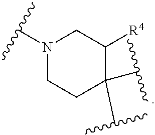

In certain embodiments, —B(R⁴)^{p1}— is

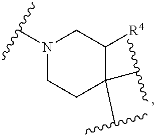

wherein R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, —B(R⁴)ₚ₁— is

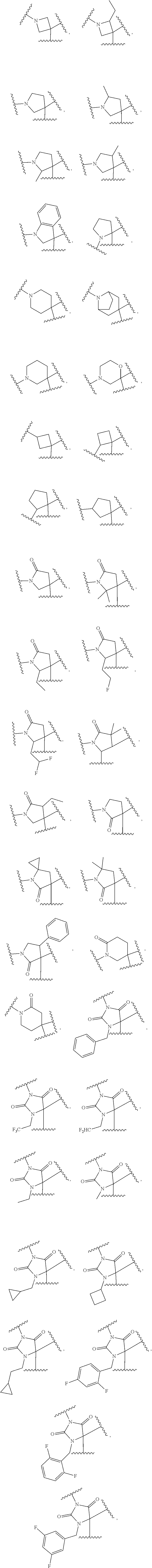

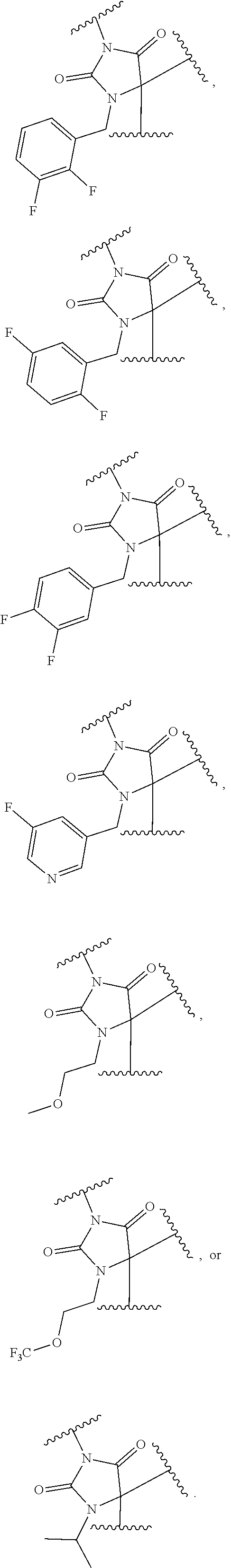

In certain embodiments, —B(R⁴)ₚ₁— is

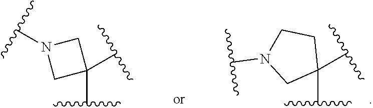 [SMILES: *N1CC(*)(*)C1 ; *N1CCC(*)(*)C1]

In certain embodiments, —B(R⁴)ₚ₁— is

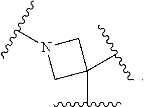 [SMILES: *N1CC(*)(*)C1]

In certain embodiments, —B(R⁴)ₚ₁— is

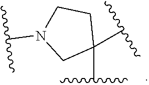 [SMILES: *N1CCC(*)(*)C1]

### R⁴

As described herein, each occurrence of R⁴ is independently halogen, substituted or unsubstituted alkyl, substituted or unsubstituted alkoxy, substituted or unsubstituted cycloalkyl, substituted or unsubstituted aryl, or two instances of R⁴ on different carbons combine to form a bridged ring, or two instances of R⁴ on the same carbon form with that carbon a carbonyl or a cycloalkyl; p1l is 0, 1, 2, 3, 4, 5, or 6; and p2 is 0, 1, 2, 3, 4, 5, or 6. In certain embodiments, each R⁴ is independently halogen, substituted or unsubstituted alkyl, or substituted or unsubstituted alkoxy, or two instances of R⁴ on the same carbon form with that carbon a carbonyl; p1 is 0, 1, 2, 3, 4, 5, or 6; and p2 is 0, 1, 2, 3, 4, 5, or 6.

In certain embodiments, each R⁴ is independently substituted or unsubstituted alkyl, or two instances of R⁴ on the same carbon form with that carbon a carbonyl; p1 is 0, 1, 2, 3, 4, or 5; and p2 is 0, 1, 2, 3, 4, or 5.

In certain embodiments, R⁴ is substituted or unsubstituted alkyl, or two instances of R⁴ on the same carbon form with that carbon a carbonyl. In certain embodiments, R⁴ is substituted or unsubstituted C₁₋₄ alkyl, or two instances of R⁴ on the same carbon form with that carbon a carbonyl. In certain embodiments, R⁴ is substituted or unsubstituted alkyl. In certain embodiments, R⁴ is substituted or unsubstituted C₁₋₄ alkyl. In certain embodiments, R⁴ is methyl, ethyl, isopropyl, benzyl, or 2,2,2-trifluoroethyl. In certain embodiments, two instances of R⁴ on the same carbon form with that carbon a carbonyl. In certain embodiments, R⁴ is methyl, ethyl, isopropyl, benzyl, or 2,2,2-trifluoroethyl, or two instances of R⁴ on the same carbon form with that carbon a carbonyl. In certain embodiments, R⁴ is haloalkyl. In certain embodiments, R⁴ is 2,2-difluoroethyl. In certain embodiments, R⁴ is 4,4,4-trifluorobutyl. In certain embodiments, R⁴ is CF₃OCH₂CH₂—. In certain embodiments, R⁴ is ethoxyethyl. In certain embodiments, R⁴ is methyl. In certain embodiments, R⁴ is ethyl. In certain embodiments, R⁴ is isopropyl. In certain embodiments, R⁴ is substituted or unsubstituted arylalkyl. In certain embodiments, R⁴ is substituted or unsubstituted benzyl. In certain embodiments, R⁴ is substituted or unsubstituted cycloalkylalkyl. In certain embodiments, R⁴ is cyclopropylethyl. In certain embodiments, R⁴ is cyclopropylmethyl. In certain embodiments, R⁴ is substituted or unsubstituted cycloalkyl. In certain embodiments, R⁴ is cyclobutyl. In certain embodiments, R⁴ is substituted or unsubstituted aryl. In certain embodiments, R⁴ is substituted or unsubstituted phenyl.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, two instances of R⁴ on different carbons combine to form a bridged ring.

In certain embodiments, p1 is 0, 1, 2, 3, 4, or 5. In certain embodiments, p1 is 0, 1, 2, 3, or 4. In certain embodiments, p1 is 0, 1, 2, or 3. In certain embodiments, p1 is 0, 1, or 2. In certain embodiments, p1 is 0 or 2. In certain embodiments, p is 0 or 1. In certain embodiments, p1 is 1 or 2. In certain embodiments, p1 is 0. In certain embodiments, p1 is 2. In certain embodiments, p1 is 1.

In certain embodiments, p2 is 0, 1, 2, 3, 4, or 5. In certain embodiments, p2 is 0, 1, 2, 3, or 4. In certain embodiments, p2 is 0, 1, 2, or 3. In certain embodiments, p2 is 0, 1, or 2. In certain embodiments, p2 is 0 or 2. In certain embodiments, p2 is 0 or 1. In certain embodiments, p2 is 1 or 2. In certain embodiments, p2 is 0. In certain embodiments, p2 is 2. In certain embodiments, p2 is 1.

In certain embodiments, p1 is 0, and p2 is 0.

m and n

As described herein, m is 0, 1, or 2; and n is 0, 1, or 2. In certain embodiments, m is 0. In certain embodiments, m is 1. In certain embodiments, m is 2. In certain embodiments, n is 0. In certain embodiments, n is 1. In certain embodiments, n is 2.

In certain embodiments, m is 0; and n is 0. In certain embodiments, m is 0; and n is 1. In certain embodiments, m is 0; and n is 2. In certain embodiments, m is 1; and n is 0. In certain embodiments, m is 1; and n is 1. In certain embodiments, m is 1; and n is 2. In certain embodiments, m is 2; and n is 0. In certain embodiments, m is 2; and n is 1. In certain embodiments, m is 2; and n is 2.

### X

As described herein, X is —O—, —NR^{A}—, or —C(R⁴)₂—. In certain embodiments, X is —O—. In certain embodiments, X is —NR^{A}—. In certain embodiments, X is —NH—. In certain embodiments, X is —C(R⁴)₂—. In certain embodiments, X is —CH(CH₃)—. In certain embodiments, X is —CH₂—. In certain embodiments, X is —C(═O)—. In certain embodiments, X is —CH(R⁴)—, wherein R⁴ combines with an R⁴ on a different carbon to form a bridged ring.

### Y¹

As described herein, Y¹ is N or CR⁵, wherein R⁵ is hydrogen, halogen, or substituted or unsubstituted alkyl. In certain embodiments, Y¹ is N. In certain embodiments, Y¹ is CR⁵. In certain embodiments, Y¹ is CH.

### L

As described herein, a bond, —SO₂—, —C(═O)—, —C(═O)C(R⁵)(R⁶)—, —NR^{A}—, or —NHC(═O)—, wherein R⁵ and R⁶ are each independently hydrogen, halogen, or substituted or unsubstituted alkyl. In certain embodiments, L is a bond, —SO₂—, —C(═O)—, —C(═O)C(R⁵)(R⁶)—, or —NHC(═O)—, wherein R⁵ and R⁶ are each independently hydrogen, halogen, or substituted or unsubstituted alkyl. In certain embodiments, R⁵ and R⁶ are each independently hydrogen or substituted or unsubstituted alkyl. In certain embodiments, R⁵ and R⁶ are each independently hydrogen or unsubstituted alkyl. In certain embodiments, R⁵ and R⁶ are each independently hydrogen or unsubstituted C₁₋₄ alkyl. In certain embodiments, R⁵ and R⁶ are each hydrogen.

In certain embodiments, L is a bond, —C(═O)—, or —C(═O)C(R⁵)(R⁶)—. In certain embodiments, L is a bond or —C(═O)—. In certain embodiments, L is a bond. In certain embodiments, L is —C(═O)—. In certain embodiments, L is —C(═O)—, and A is

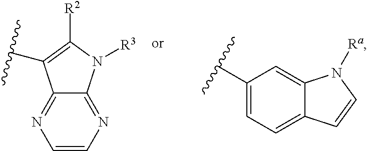

wherein R^{a} is hydrogen or substituted or unsubstituted alkyl. In certain embodiments, L is —C(═O)—, and A is

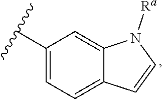

wherein R^{a} is hydrogen or substituted or unsubstituted alkyl. In certain embodiments, L is —C(═O)—, and A is

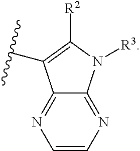

In certain embodiments, L is —C(═O)C(R⁵)(R⁶)—. In certain embodiments, L is C(═O)C(R⁵)(R⁶)—, and A is substituted or unsubstituted heteroaryl. In certain embodiments, L is —C(═O)CH₂—. In certain embodiments, L is —C(═O)CH₂—, and A is substituted or unsubstituted heteroaryl. In certain embodiments, L is —NHC(═O)—. In certain embodiments, L is —SO₂—. In certain embodiments, L is —NR^{A}-. In certain embodiments, L is —NH—.

### A

As described herein, A is

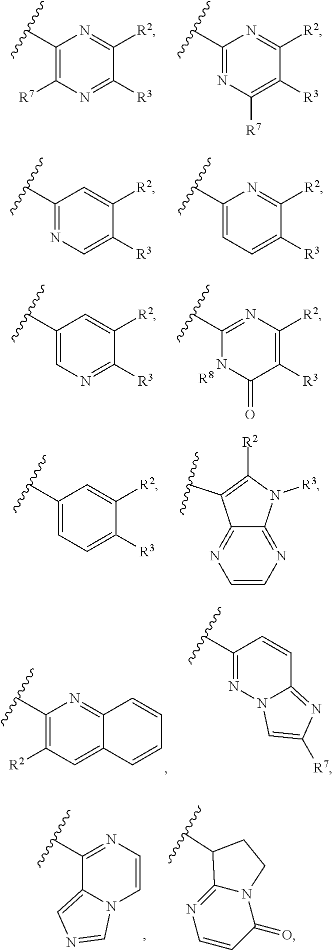

or substituted or unsubstituted 5-membered heteroaryl; wherein R² and R³ are each independently hydrogen, halogen, substituted or unsubstituted alkyl, substituted or unsubstituted alkoxy, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl; R⁷ is hydrogen, substituted or unsubstituted alkyl, or substituted or unsubstituted alkoxy; and R⁸ is substituted or unsubstituted alkyl.

In certain embodiments, A is

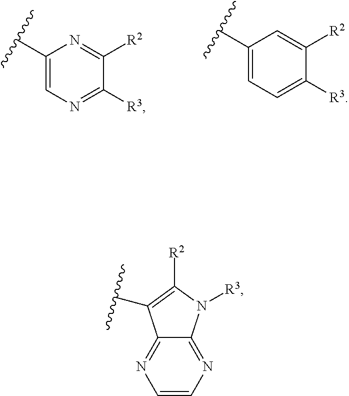

or substituted or unsubstituted 5-membered heteroaryl; wherein R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl.

In certain embodiments, A is

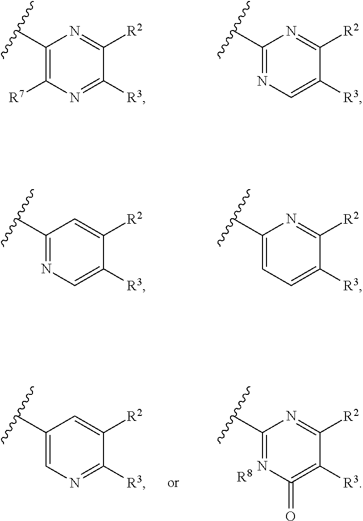

In certain embodiments, A is

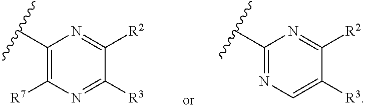

In certain embodiments, A is

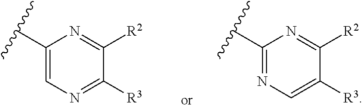

In certain embodiments, A is

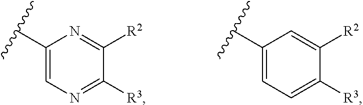

or substituted or unsubstituted 5-membered heteroaryl.

In certain embodiments, A is substituted or unsubstituted 5-membered heteroaryl. In certain embodiments, A is substituted or unsubstituted 5-membered heteroaryl comprising at least one nitrogen in the heteroaryl ring. In certain embodiments, A is substituted or unsubstituted pyrazolyl, substituted or unsubstituted imidazolyl, substituted or unsubstituted pyrrolyl, substituted or unsubstituted triazolyl, substituted or unsubstituted tetrazolyl, substituted or unsubstituted oxazolyl, substituted or unsubstituted isoxazolyl, substituted or unsubstituted oxadiazolyl, substituted or unsubstituted thiazolyl, or substituted or unsubstituted thiadiazolyl. In certain embodiments, A is substituted or unsubstituted pyrazolyl, substituted or unsubstituted triazolyl, substituted or unsubstituted isoxazolyl, or substituted or unsubstituted thiazolyl. In certain embodiments, A is substituted or unsubstituted pyrazolyl, or substituted or unsubstituted thiazolyl. In certain embodiments, A is substituted pyrazolyl or substituted thiazolyl. In certain embodiments, A is

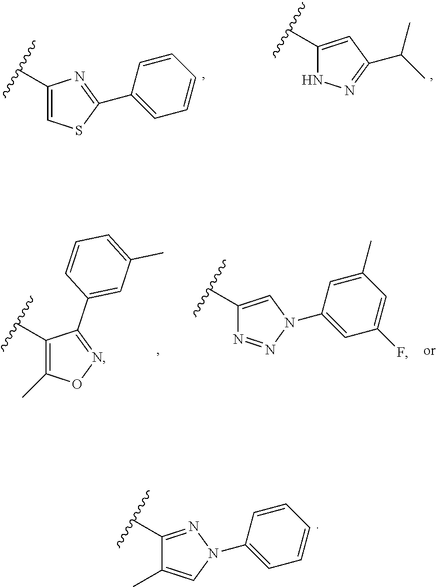

In certain embodiments, A is

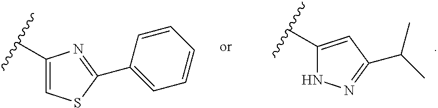

In certain embodiments, A is

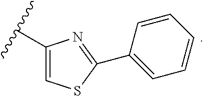 [SMILES: *c1csc(-c2ccccc2)n1]

In certain embodiments, A is

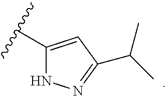 [SMILES: *c1cc(C(C)C)n[nH]1]

In certain embodiments, A is

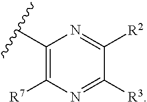

In certain embodiments, A is

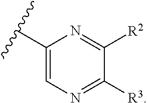

In certain embodiments, A is

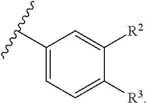

In certain embodiments, A is

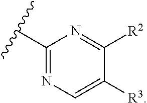

In certain embodiments, A is

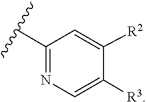

In certain embodiments, A is

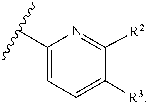

In certain embodiments, A is

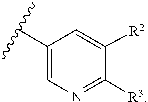

In certain embodiments, A is

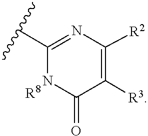

In certain embodiments, R² and R³ are each independently hydrogen or substituted or unsubstituted heteroaryl; or R² and R³ together with the atoms to which they are attached form a substituted or unsubstituted aryl, or substituted or unsubstituted heteroaryl. In certain embodiments, R² and R³ are each independently hydrogen or substituted or unsubstituted heteroaryl; or R² and R³ together with the atoms to which they are attached form a substituted or unsubstituted heterocyclyl, or substituted or unsubstituted heteroaryl.

In certain embodiments, R² is substituted or unsubstituted heteroaryl. In certain embodiments, R² is unsubstituted heteroaryl. In certain embodiments, R² is substituted or unsubstituted thiadizaolyl. In certain embodiments, R² is unsubstituted thiadizaolyl.

In certain embodiments, R³ is hydrogen.

In certain embodiments, R² is substituted or unsubstituted heteroaryl; and R³ is hydrogen. In certain embodiments, R² is unsubstituted heteroaryl; and R³ is hydrogen. In certain embodiments, R² is substituted or unsubstituted thiadizaolyl; and R³ is hydrogen. In certain embodiments, R² is unsubstituted thiadizaolyl; and R³ is hydrogen.

In certain embodiments, R² and R³ together with the atoms to which they are attached form a substituted or unsubstituted aryl, or substituted or unsubstituted heteroaryl. In certain embodiments, R² and R³ together with the atoms to which they are attached form a substituted or unsubstituted heterocyclyl, or substituted or unsubstituted heteroaryl.

In certain embodiments, R² and R³ together with the atoms to which they are attached form a substituted or unsubstituted aryl. In certain embodiments, R² and R³ together with the atoms to which they are attached form a substituted or unsubstituted phenyl. In certain embodiments, R² and R³ together with the atoms to which they are attached form a substituted phenyl. In certain embodiments, R² and R³ together with the atoms to which they are attached form an unsubstituted phenyl.

In certain embodiments, R² and R³ together with the atoms to which they are attached form a substituted or unsubstituted heteroaryl. In certain embodiments, R² and R³ together with the atoms to which they are attached form a substituted or unsubstituted pyrrolyl or substituted or unsubstituted pyrazolyl. In certain embodiments, R² and R³ together with the atoms to which they are attached form a substituted pyrrolyl or substituted pyrazolyl. In certain embodiments, R² and R³ together with the atoms to which they are attached form a substituted pyrrolyl or substituted pyrazolyl, wherein the pyrrolyl or pyrazolyl is substituted with substituted or unsubstituted alkyl, or substituted or unsubstituted heterocyclyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclyl, haloalkyl, or substituted or unsubstituted heterocyclylalkyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclyl, substituted or unsubstituted heterocyclylalkyl, or C₁₋₄ haloalkyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclyl. In certain embodiments, R² and R³ together with the atoms to which they are attached form a substituted pyrrolyl or substituted pyrazolyl, wherein the pyrrolyl or pyrazolyl is substituted with C₁₋₄ haloalkyl. In certain embodiments, R² and R³ together with the atoms to which they are attached form a substituted pyrrolyl or substituted pyrazolyl, wherein the pyrrolyl or pyrazolyl is substituted with substituted or unsubstituted heterocyclylalkyl.

In certain embodiments, R² and R³ together with the atoms to which they are attached form a substituted or unsubstituted pyrazolyl. In certain embodiments, R² and R³ together with the atoms to which they are attached form a substituted pyrazolyl. In certain embodiments, R² and R³ together with the atoms to which they are attached form a substituted pyrazolyl, wherein the pyrazolyl is substituted with substituted or unsubstituted alkyl, or substituted or unsubstituted heterocyclyl. In certain embodiments, R² and R³ together with the atoms to which they are attached form a substituted pyrazolyl, wherein the pyrazolyl is substituted with substituted or unsubstituted heterocyclyl. In certain embodiments, R² and R³ together with the atoms to which they are attached form a substituted pyrazolyl, wherein the pyrazolyl is substituted with substituted or unsubstituted 4-5 membered heterocyclyl. In certain embodiments, R² and R³ together with the atoms to which they are attached form a substituted pyrazolyl, wherein the pyrazolyl is substituted with substituted or unsubstituted oxetanyl. In certain embodiments, R² and R³ together with the atoms to which they are attached form a substituted pyrazolyl, wherein the pyrazolyl is substituted with unsubstituted alkyl or haloalkyl. In certain embodiments, R² and R³ together with the atoms to which they are attached form a substituted pyrazolyl, wherein the pyrazolyl is substituted with unsubstituted C₁₋₄ alkyl or C₁₋₄ haloalkyl. In certain embodiments, R² and R³ together with the atoms to which they are attached form a substituted pyrazolyl, wherein the pyrazolyl is substituted with unsubstituted C₁₋₄ alkyl. In certain embodiments, R² and R³ together with the atoms to which they are attached form a substituted pyrazolyl, wherein the pyrazolyl is substituted with C₁₋₄ haloalkyl.

In certain embodiments, R² and R³ together with the atoms to which they are attached form a substituted or unsubstituted pyrrolyl. In certain embodiments, R² and R³ together with the atoms to which they are attached form a substituted pyrrolyl. In certain embodiments, R² and R³ together with the atoms to which they are attached form a substituted pyrrolyl, wherein the pyrrolyl is substituted with substituted or unsubstituted alkyl. In certain embodiments, R² and R³ together with the atoms to which they are attached form a substituted pyrrolyl, wherein the pyrrolyl is substituted with unsubstituted alkyl or haloalkyl. In certain embodiments, R² and R³ together with the atoms to which they are attached form a substituted pyrrolyl, wherein the pyrrolyl is substituted with unsubstituted C₁₋₄ alkyl or C₁₋₄ haloalkyl. In certain embodiments, R² and R³ together with the atoms to which they are attached form a substituted pyrrolyl, wherein the pyrrolyl is substituted with unsubstituted C₁₋₄ alkyl. In certain embodiments, R² and R³ together with the atoms to which they are attached form a substituted pyrrolyl, wherein the pyrrolyl is substituted with C₁₋₄ haloalkyl. In certain embodiments, R² and R³ together with the atoms to which they are attached form a substituted pyrrolyl, wherein the pyrrolyl is substituted with substituted or unsubstituted heterocyclylalkyl. In certain embodiments, R² and R³ together with the atoms to which they are attached form a substituted pyrrolyl, wherein the pyrrolyl is substituted with substituted or unsubstituted oxetanylalkyl.

In certain embodiments, R² and R³ together with the atoms to which they are attached form a substituted or unsubstituted heterocyclyl. In certain embodiments, R² and R³ together with the atoms to which they are attached form a substituted or unsubstituted dihydroimidazol-2-one. In certain embodiments R² and R³ together with the atoms to which they are attached form

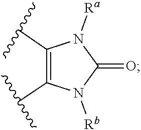

wherein R^{a} is substituted or unsubstituted alkyl or substituted or unsubstituted heterocyclyl; and R^{b} is substituted or unsubstituted alkyl or substituted or unsubstituted heterocyclyl.

In certain embodiments, A is

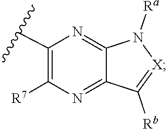

wherein R⁷ is hydrogen, unsubstituted C₁₋₄ alkyl, or unsubstituted C₁₋₄ alkoxy; X is N or CR^{a}; each R^{a} is independently hydrogen, substituted or unsubstituted alkyl, substituted or unsubstituted aryl, or substituted or unsubstituted heterocyclyl; and R^{b} is hydrogen, substituted or unsubstituted alkyl, or substituted or unsubstituted alkoxy.

In certain embodiments, A is

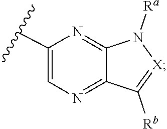

wherein X is N or CR^{a}; each R^{a} is independently hydrogen, substituted or unsubstituted alkyl, or substituted or unsubstituted heterocyclyl; and R^{b} is hydrogen, halogen, substituted or unsubstituted alkyl, or substituted or unsubstituted alkoxy. In certain embodiments, R^{a} is independently hydrogen, or substituted or unsubstituted alkyl; and R^{b} is hydrogen, halogen, substituted or unsubstituted alkyl, or substituted or unsubstituted alkoxy. In certain embodiments, R^{a} is independently hydrogen, or substituted or unsubstituted alkyl; and R^{b} is halogen, substituted or unsubstituted alkyl, or substituted or unsubstituted alkoxy. In certain embodiments, R^{a} is independently hydrogen, or substituted or unsubstituted alkyl; and R^{b} is substituted or unsubstituted alkyl, or substituted or unsubstituted alkoxy. In certain embodiments, X is N or CH; R^{a} is hydrogen, or substituted or unsubstituted alkyl; and R^{b} is substituted or unsubstituted alkyl, or substituted or unsubstituted alkoxy. In certain embodiments, X is N; R^{a} is hydrogen, or substituted or unsubstituted alkyl; and R^{b} is substituted or unsubstituted alkyl, or substituted or unsubstituted alkoxy. In certain embodiments, X is N; R^{a} is hydrogen, or substituted or unsubstituted alkyl; and R^{b} is hydrogen, substituted or unsubstituted alkyl, or substituted or unsubstituted alkoxy. In certain embodiments, X is N; R^{a} is hydrogen, or substituted or unsubstituted alkyl; and R^{b} is hydrogen, substituted or unsubstituted C₁₋₄ alkyl, or substituted or unsubstituted C₁₋₄ alkoxy. In certain embodiments, X is N; R^{a} is hydrogen, or substituted or unsubstituted alkyl; and R^{b} is substituted or unsubstituted C₁₋₄ alkyl, or substituted or unsubstituted C₁₋₄ alkoxy. In certain embodiments, X is N; R^{a} is hydrogen, or substituted or unsubstituted alkyl; and R^{b} is unsubstituted C₁₋₄ alkyl, C₁₋₄ haloalkyl, C₁₋₄ haloalkoxy, or unsubstituted C₁₋₄ alkoxy. In certain embodiments, X is N; R^{a} is hydrogen, or substituted or unsubstituted alkyl; and R^{b} is hydrogen, unsubstituted C₁₋₄ alkyl, C₁₋₄ haloalkyl, C₁₋₄ haloalkoxy, or unsubstituted C₁₋₄ alkoxy. In certain embodiments, X is CH; R^{a} is hydrogen, or substituted or unsubstituted alkyl; and R^{b} is substituted or unsubstituted alkyl, or substituted or unsubstituted alkoxy.

In certain embodiments, R^{b} is hydrogen, halogen, substituted or unsubstituted alkyl, substituted or unsubstituted cycloalkyl, or substituted or unsubstituted alkoxy. In certain embodiments, R^{b} is halogen, substituted or unsubstituted alkyl, substituted or unsubstituted cycloalkyl, or substituted or unsubstituted alkoxy. In certain embodiments, R^{b} is substituted or unsubstituted alkyl, substituted or unsubstituted cycloalkyl, or substituted or unsubstituted alkoxy. In certain embodiments, R^{b} is substituted or unsubstituted alkyl, substituted or unsubstituted cycloalkyl, or substituted or unsubstituted alkoxy. In certain embodiments, R^{b} is hydrogen, substituted or unsubstituted alkyl, substituted or unsubstituted cycloalkyl, or substituted or unsubstituted alkoxy. In certain embodiments, R^{b} is hydrogen, substituted or unsubstituted C₁₋₄ alkyl, substituted or unsubstituted C₃₋₄ cycloalkyl, or substituted or unsubstituted C₁₋₄ alkoxy. In certain embodiments, R^{b} is substituted or unsubstituted C₁₋₄ alkyl, substituted or unsubstituted C₃₋₄ cycloalkyl, or substituted or unsubstituted C₁₋₄ alkoxy. In certain embodiments, R^{b} is unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, R^{b} is hydrogen, unsubstituted C₁₋₄ alkyl, C₁₋₄ haloalkyl, unsubstituted C₃₋₄ cycloalkyl, unsubstituted C₃₋₄ cycloalkylmethyl, C₁₋₄ haloalkoxy, or unsubstituted C₁₋₄ alkoxy. In certain embodiments, R^{b} is hydrogen.

In certain embodiments, A is

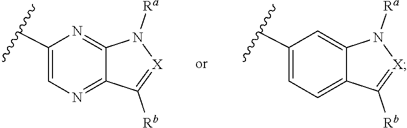

wherein R^{a} is hydrogen or substituted or unsubstituted alkyl; and R^{b} is hydrogen, halogen, substituted or unsubstituted alkyl, or substituted or unsubstituted alkoxy.

In certain embodiments, A is

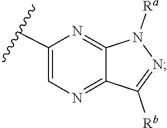

wherein R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, haloalkoxyalkyl, heterocyclyalkyl, arylalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is 2,2-difluoroethyl or oxetanyl; and R^{b} is hydrogen, —CH₃, or —OCH₃.

In certain embodiments, A is

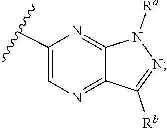

wherein R^{a} is hydrogen or substituted or unsubstituted alkyl; and R^{b} is hydrogen, halogen, substituted or unsubstituted alkyl, or substituted or unsubstituted alkoxy.

In certain embodiments, A is

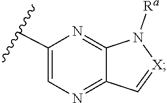

wherein X is N or CR^{a}; and each R^{a} is independently hydrogen, substituted or unsubstituted alkyl, or substituted or unsubstituted heterocyclyl. In certain embodiments, each R^{a} is independently hydrogen, or substituted or unsubstituted alkyl. In certain embodiments, X is N or CH. In certain embodiments, X is N. In certain embodiments, X is CH.

In certain embodiments, A is

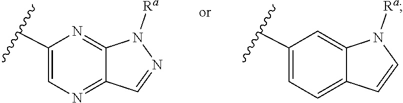

wherein R^{a} is hydrogen, or substituted or unsubstituted alkyl.

In certain embodiments, A is

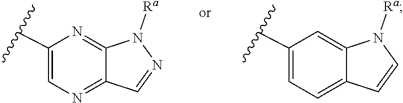

wherein R^{a} is hydrogen, heterocyclylalkyl, haloalkyl, or substituted or unsubstituted 4-5 membered heterocyclyl.

In certain embodiments, A is

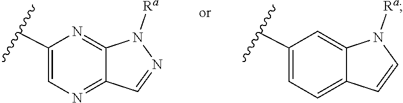

wherein R^{a} is hydrogen or haloalkyl.

In certain embodiments, A is

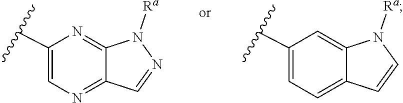

wherein R^{a} is hydrogen or C₁₋₄ haloalkyl.

In certain embodiments, A is

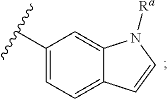

wherein R^{a} is hydrogen, or substituted or unsubstituted alkyl.

In certain embodiments, A is

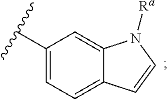

wherein R^{a} is hydrogen.

In certain embodiments, A is

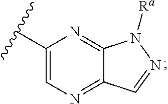

wherein R^{a} is substituted or unsubstituted alkyl.

In certain embodiments, A is

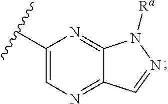

wherein R^{a} is haloalkyl.

In certain embodiments, A is

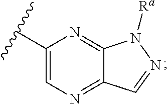

wherein R^{a} is C₁₋₄ haloalkyl.

In certain embodiments, A is

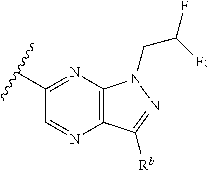

and R^{b} is hydrogen, halogen, substituted or unsubstituted alkyl, or substituted or unsubstituted alkoxy. In certain embodiments, R^{b} is halogen, substituted or unsubstituted alkyl, or substituted or unsubstituted alkoxy. In certain embodiments, R^{b} is substituted or unsubstituted alkyl, or substituted or unsubstituted alkoxy. In certain embodiments, R^{b} is hydrogen, substituted or unsubstituted alkyl, or substituted or unsubstituted alkoxy. In certain embodiments, R^{b} is hydrogen, substituted or unsubstituted C₁₋₄ alkyl, or substituted or unsubstituted C₁₋₄ alkoxy. In certain embodiments, R^{b} is substituted or unsubstituted C₁₋₄ alkyl, or substituted or unsubstituted C₁₋₄ alkoxy. In certain embodiments, R^{b} is unsubstituted C₁₋₄ alkyl, C₁₋₄ haloalkyl, C₁₋₄ haloalkoxy, or unsubstituted C₁₋₄ alkoxy. In certain embodiments, R^{b} is hydrogen, unsubstituted C₁₋₄ alkyl, C₁₋₄ haloalkyl, C₁₋₄ haloalkoxy, or unsubstituted C₁₋₄ alkoxy.

In certain embodiments, A is

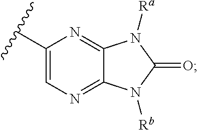

wherein R^{a} is substituted or unsubstituted alkyl or substituted or unsubstituted heterocyclyl; and R^{b} is substituted or unsubstituted alkyl or substituted or unsubstituted heterocyclyl.

In certain embodiments, R^{a} is unsubstituted alkyl, haloalkyl, or unsubstituted heterocyclyl; and R^{b} is unsubstituted alkyl, haloalkyl, or unsubstituted heterocyclyl. In certain embodiments, R^{a} is unsubstituted C₁₋₄ alkyl, C₁₋₄ haloalkyl, or unsubstituted 4-5 membered heterocyclyl; and R^{b} is unsubstituted C₁₋₄ alkyl, C₁₋₄ haloalkyl, or unsubstituted 4-5 membered heterocyclyl. In certain embodiments, R^{a} is C₁₋₄ haloalkyl or unsubstituted 4-5 membered heterocyclyl; and R^{b} is unsubstituted C₁₋₄ alkyl. In certain embodiments, R^{a} is unsubstituted C₁₋₄ alkyl; and R^{b} is C₁₋₄ haloalkyl or unsubstituted 4-5 membered heterocyclyl.

In certain embodiments, R^{a} is 2,2-difluoroethyl, oxetanyl, —CH₃, or —CH₂CH₃; and R^{b} is 2,2-difluoroethyl, oxetanyl, —CH₃, or —CH₂CH₃. In certain embodiments, R^{a} is 2,2-difluoroethyl or oxetanyl; and R^{b} is —CH₃, or —CH₂CH₃. In certain embodiments, R^{b} is 2,2-difluoroethyl or oxetanyl; and R^{a} is —CH₃, or —CH₂CH₃.

In certain embodiments, A is

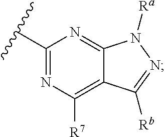

wherein R⁷ is hydrogen, unsubstituted C₁₋₄ alkyl, or unsubstituted C₁₋₄ alkoxy; R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R⁷ is hydrogen or unsubstituted C₁₋₄ alkoxy; R^{a} is substituted or unsubstituted alkyl or substituted or unsubstituted heterocyclyl; and R^{b} is hydrogen, substituted or unsubstituted alkyl, or substituted or unsubstituted alkoxy.

In certain embodiments, A is

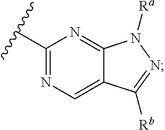

wherein R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is C₁₋₄ haloalkyl or unsubstituted 4-5 membered heterocyclyl; and R^{b} is hydrogen or unsubstituted C₁₋₄ alkyl. In certain embodiments, R^{a} is 2,2-difluoroethyl or oxetanyl; and R^{b} is hydrogen, —CH₃, or —OCH₃. In certain embodiments, R^{a} is 2,2-difluoroethyl or oxetanyl; and R^{b} is hydrogen or —CH₃.

In certain embodiments, A is

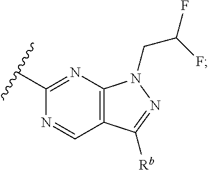

and R^{b} is hydrogen, halogen, substituted or unsubstituted alkyl, or substituted or unsubstituted alkoxy. In certain embodiments, R^{b} is halogen, substituted or unsubstituted alkyl, or substituted or unsubstituted alkoxy. In certain embodiments, R^{b} is substituted or unsubstituted alkyl, or substituted or unsubstituted alkoxy. In certain embodiments, R^{b} is hydrogen, substituted or unsubstituted alkyl, or substituted or unsubstituted alkoxy. In certain embodiments, R^{b} is hydrogen, substituted or unsubstituted C₁₋₄ alkyl, or substituted or unsubstituted C₁₋₄ alkoxy. In certain embodiments, R^{b} is substituted or unsubstituted C₁₋₄ alkyl, or substituted or unsubstituted C₁₋₄ alkoxy. In certain embodiments, R^{b} is unsubstituted C₁₋₄ alkyl, C₁₋₄ haloalkyl, C₁₋₄ haloalkoxy, or unsubstituted C₁₋₄ alkoxy. In certain embodiments, R^{b} is hydrogen, unsubstituted C₁₋₄ alkyl, C₁₋₄ haloalkyl, C₁₋₄ haloalkoxy, or unsubstituted C₁₋₄ alkoxy. In certain embodiments, R^{b} is hydrogen or unsubstituted C₁₋₄ alkyl.

In certain embodiments, A is

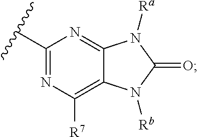

wherein R⁷ is hydrogen, unsubstituted C₁₋₄ alkyl, or unsubstituted C₁₋₄ alkoxy; R^{a} is substituted or unsubstituted alkyl or substituted or unsubstituted heterocyclyl; and R^{b} is substituted or unsubstituted alkyl or substituted or unsubstituted heterocyclyl. In certain embodiments, R⁷ is hydrogen or unsubstituted C₁₋₄ alkoxy; R^{a} is substituted or unsubstituted alkyl or substituted or unsubstituted heterocyclyl; and R^{b} is substituted or unsubstituted alkyl.

In certain embodiments, A is

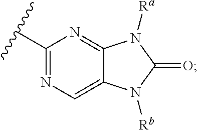

wherein R^{a} is substituted or unsubstituted alkyl or substituted or unsubstituted heterocyclyl; and R^{b} is substituted or unsubstituted alkyl or substituted or unsubstituted heterocyclyl. In certain embodiments, R^{a} is substituted or unsubstituted alkyl or substituted or unsubstituted heterocyclyl; and R^{b} is substituted or unsubstituted alkyl. In certain embodiments, R^{a} is substituted or unsubstituted alkyl; and R^{b} is substituted or unsubstituted alkyl. In certain embodiments, R^{a} is haloalkyl or heterocyclyl; and R^{b} is unsubstituted alkyl. In certain embodiments, R^{a} is haloalkyl; and R^{b} is unsubstituted alkyl. In certain embodiments, R^{a} is C₁₋₄ haloalkyl or unsubstituted 4-5 membered heterocyclyl; and R^{b} is unsubstituted C₁₋₄ alkyl. In certain embodiments, R^{a} is C₁₋₄ haloalkyl; and R^{b} is unsubstituted C₁₋₄ alkyl. In certain embodiments, R^{a} is 2,2-difluoroethyl or oxetanyl; and R^{b} is —CH₃, or —CH₂CH₃. In certain embodiments, R^{a} is 2,2-difluoroethyl; and R^{b} is —CH₃ or —CH₂CH₃.

In certain embodiments, A is

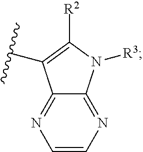

wherein R² and R³ are each independently substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, A is

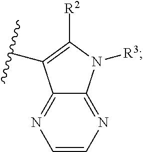

wherein R² and R³ are each independently substituted or unsubstituted alkyl, or substituted or unsubstituted aryl. In certain embodiments, A is

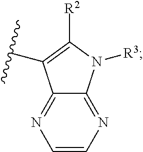

wherein R² is substituted or unsubstituted alkyl, and R³ is substituted or unsubstituted aryl. In certain embodiments, A is

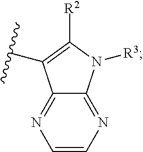

wherein R² is substituted or unsubstituted C₁₋₄ alkyl, and R³ is substituted or unsubstituted phenyl. In certain embodiments, A is

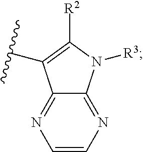

wherein R² is unsubstituted C₁₋₄ alkyl, and R³ is unsubstituted phenyl. In certain embodiments, A is

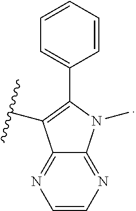 [SMILES: *c1c(-c2ccccc2)n(C)c2nccnc12 ; "."]

In certain embodiments, A is

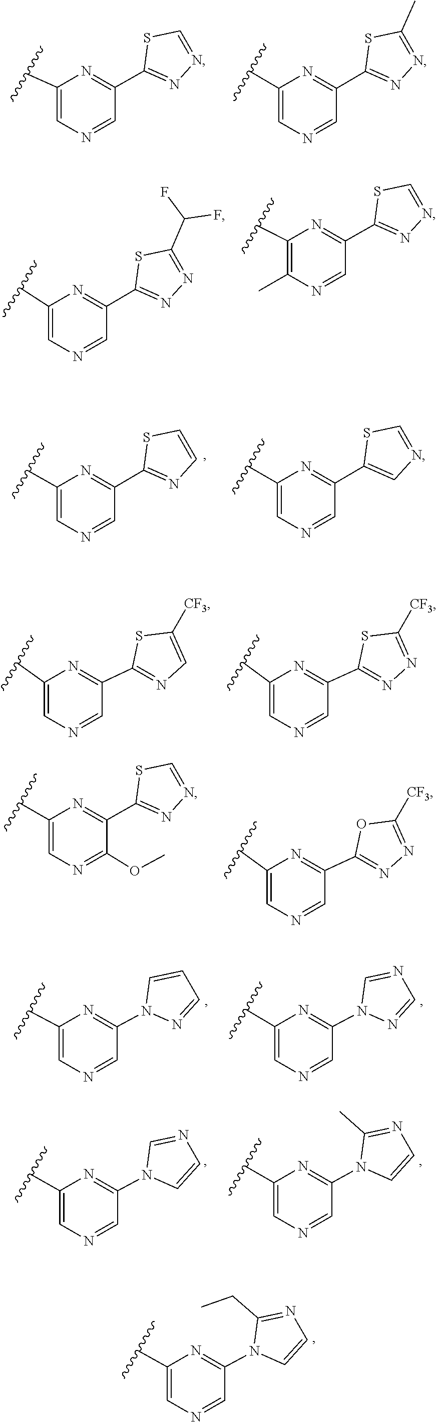

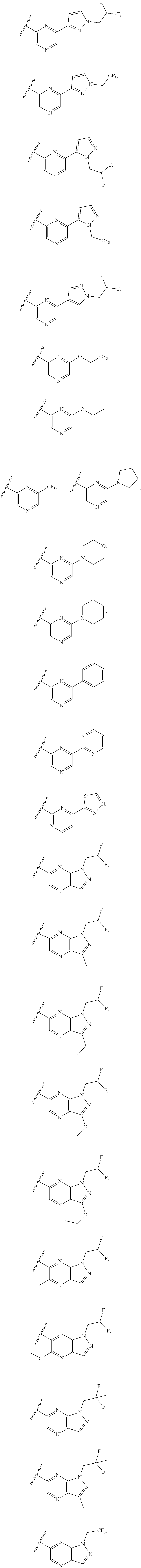

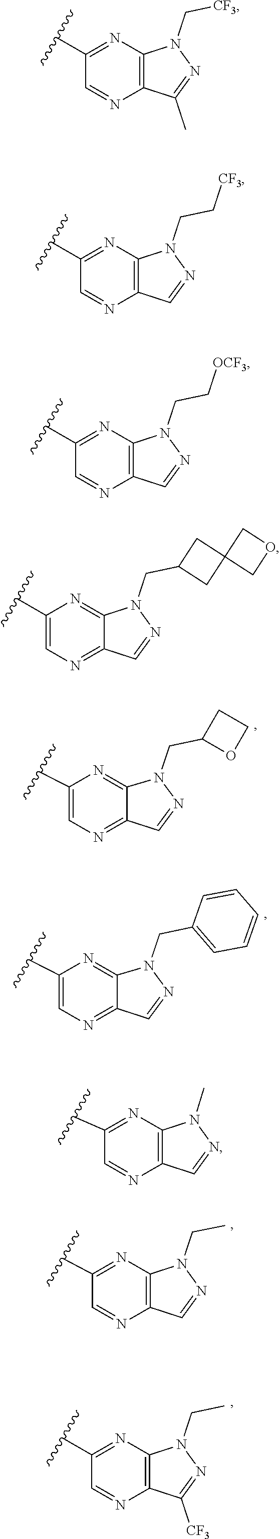

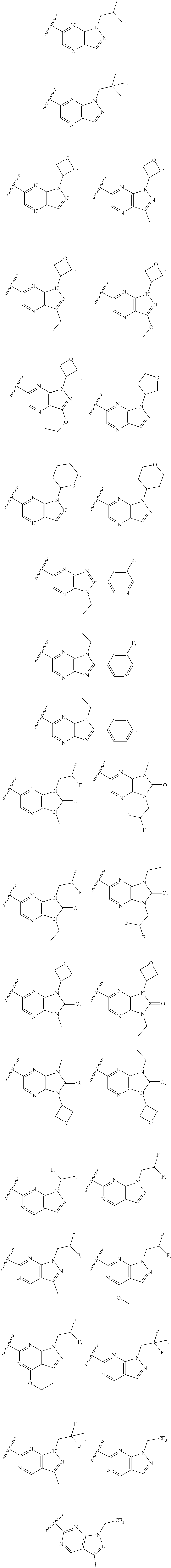

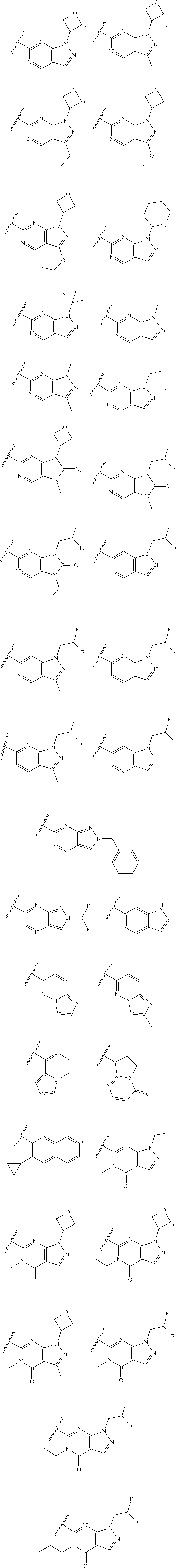

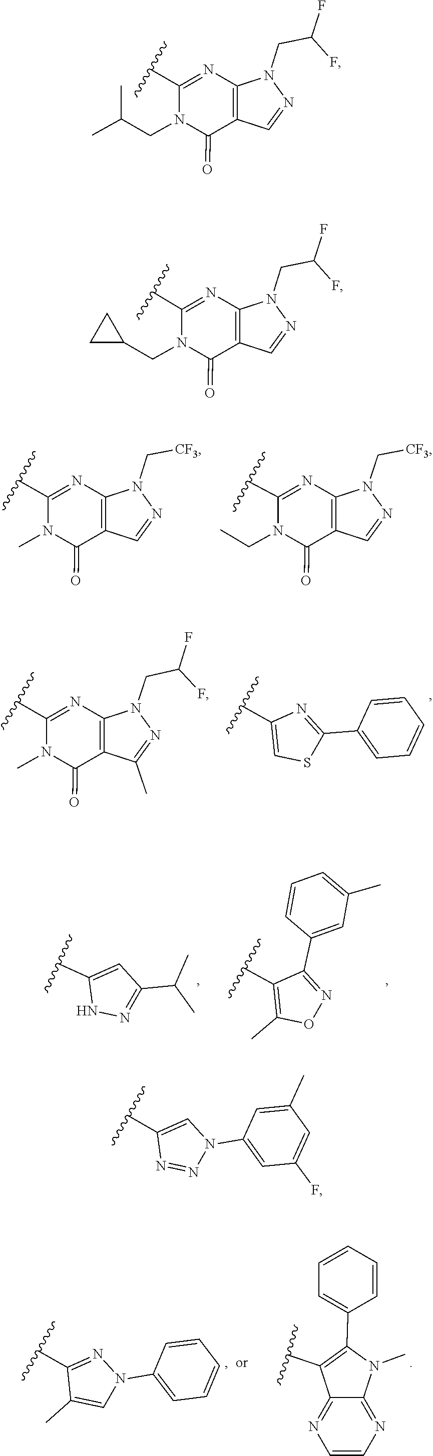

In certain embodiments, A is

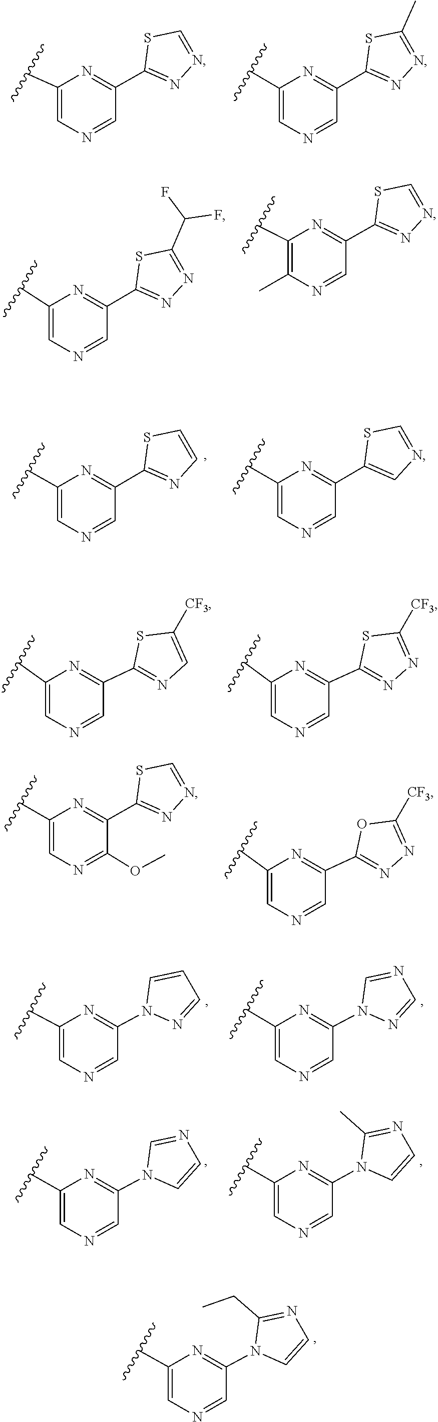

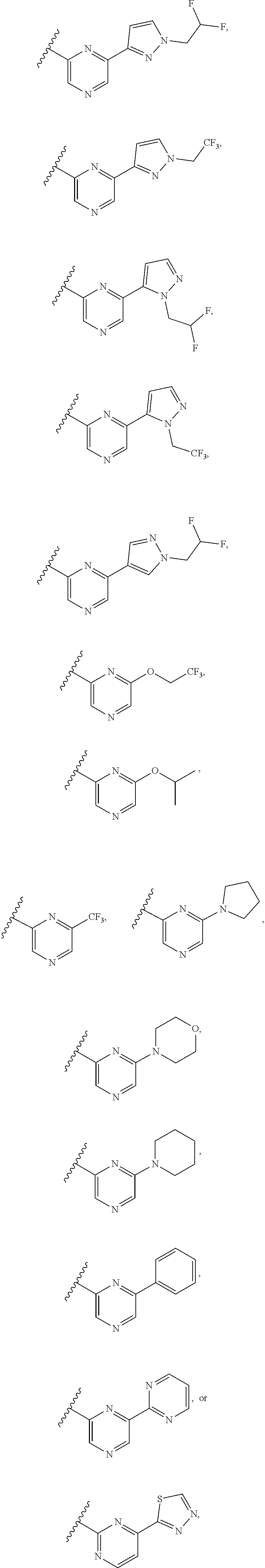

In certain embodiments, A is

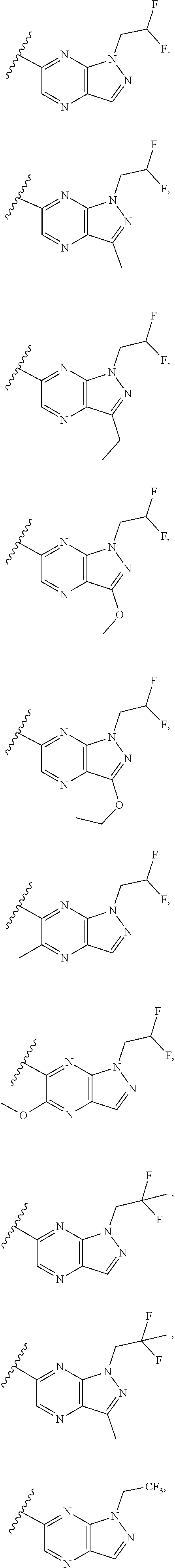

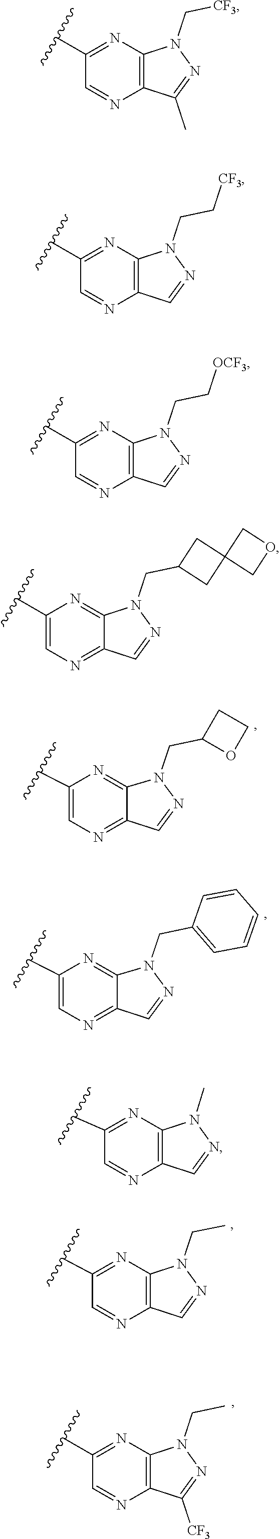

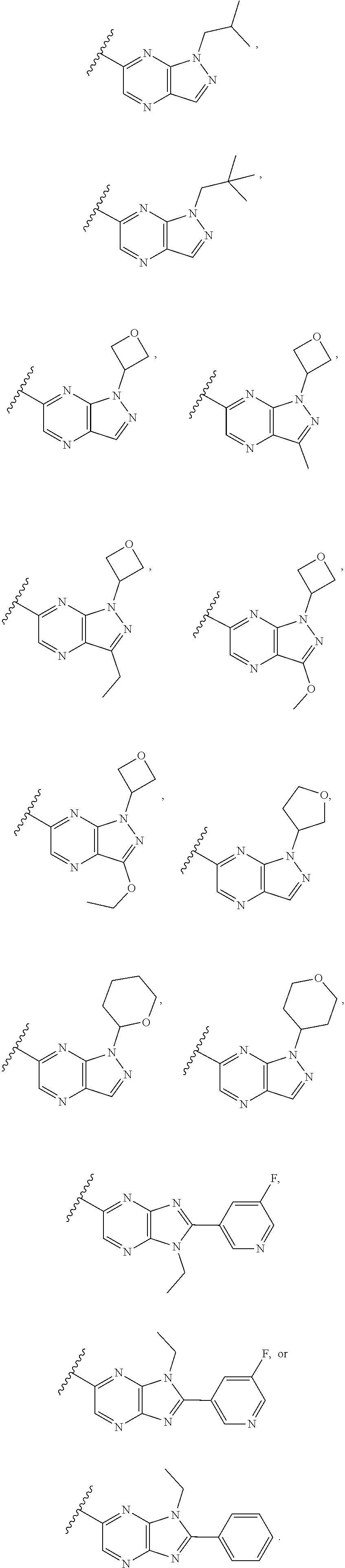

In certain embodiments, A is

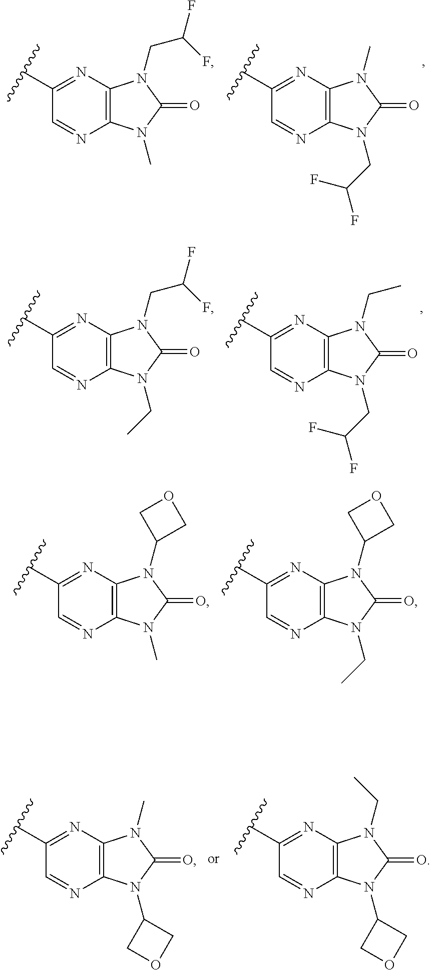

In certain embodiments, A is

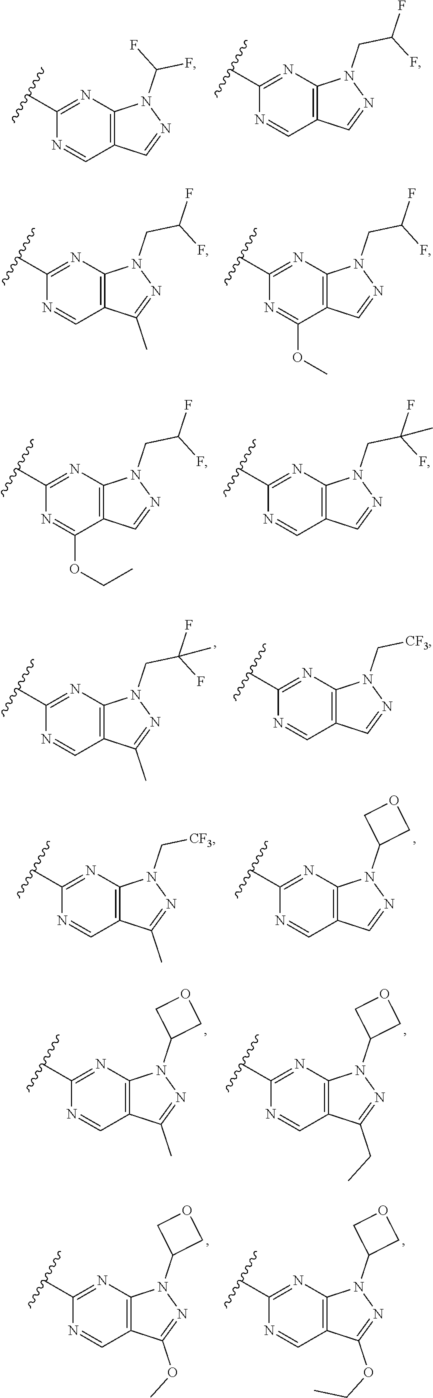

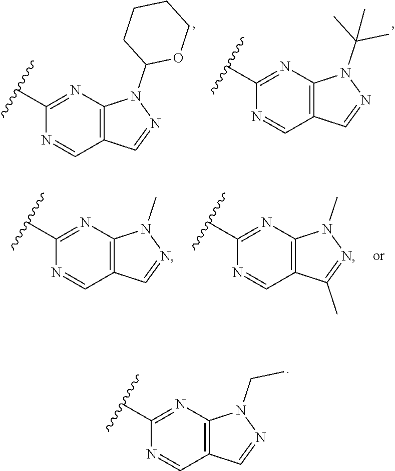 [SMILES: *c1ncc2cnn(C3CCCCO3)c2n1 ; *c1ncc2cnn(C(C)(C)C)c2n1 ; *c1ncc2cnn(C)c2n1 ; *c1ncc2c(C)nn(C)c2n1 ; *c1ncc2cnn(CC)c2n1]

In certain embodiments, A is

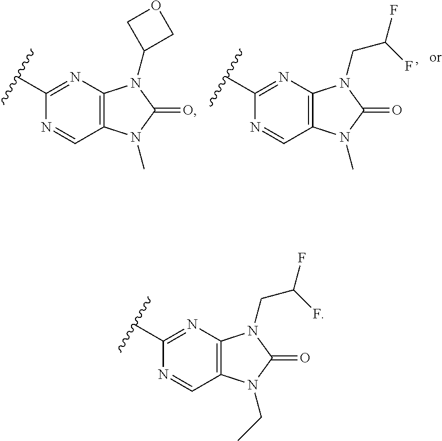 [SMILES: *c1ncc2c(n1)n(C1COC1)c(=O)n2C ; *c1ncc2c(n1)n(CC(F)F)c(=O)n2C ; "," ; "or" ; *c1ncc2c(n1)n(CC(F)F)c(=O)n2CC]

In certain embodiments, A is

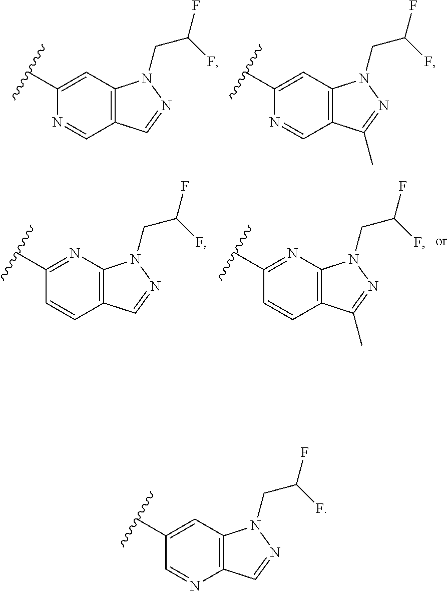

In certain embodiments, A is

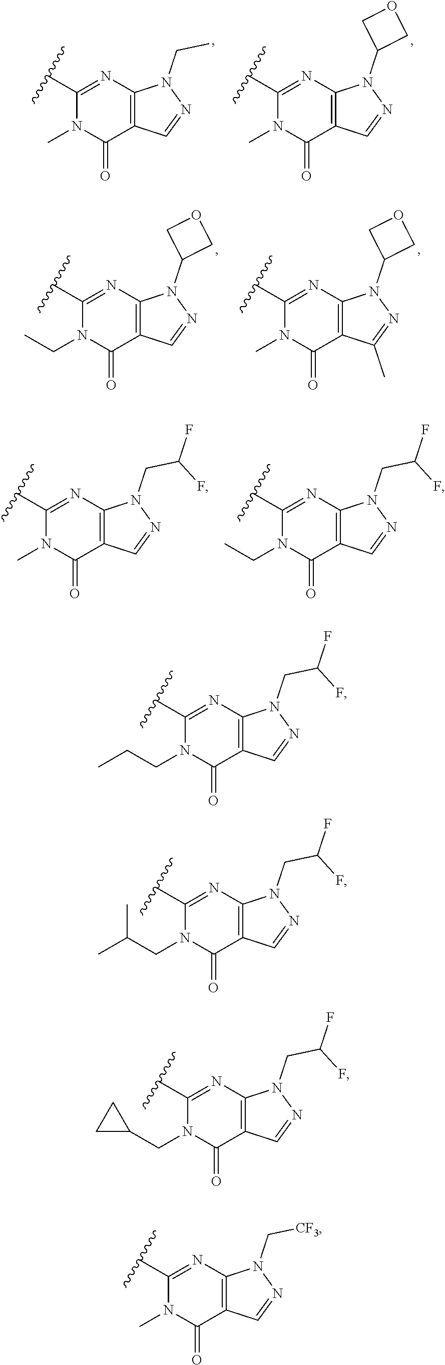

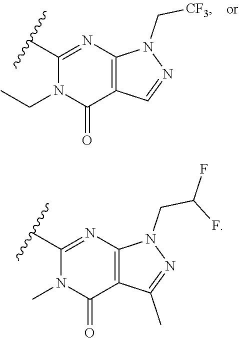 [SMILES: *c1nc2c(cnn2CC(F)(F)F)c(=O)n1CC ; *c1nc2c(c(C)nn2CC(F)F)c(=O)n1C]

In certain embodiments, A is

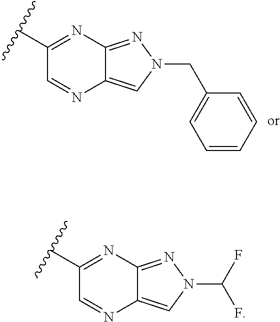 [SMILES: *c1cnc2cn(Cc3ccccc3)nc2n1 ; "or" ; *c1cnc2cn(C(F)F)nc2n1]

In certain embodiments, A is

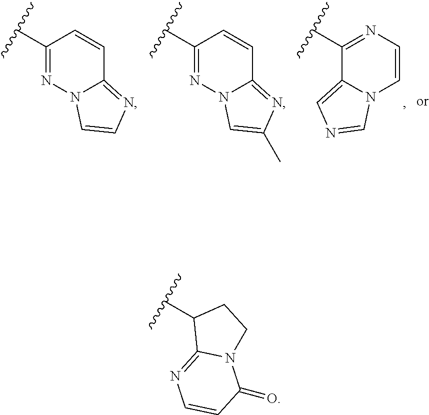

In certain embodiments, A is

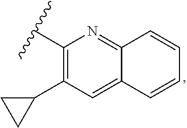 [SMILES: *c1nc2ccccc2cc1C1CC1 ; ","]

In certain embodiments, A is

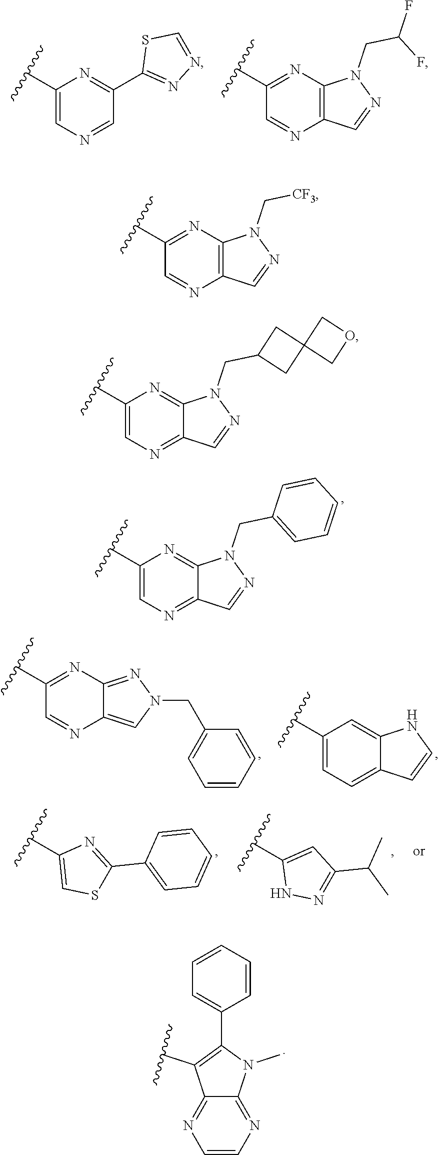

In certain embodiments, A is

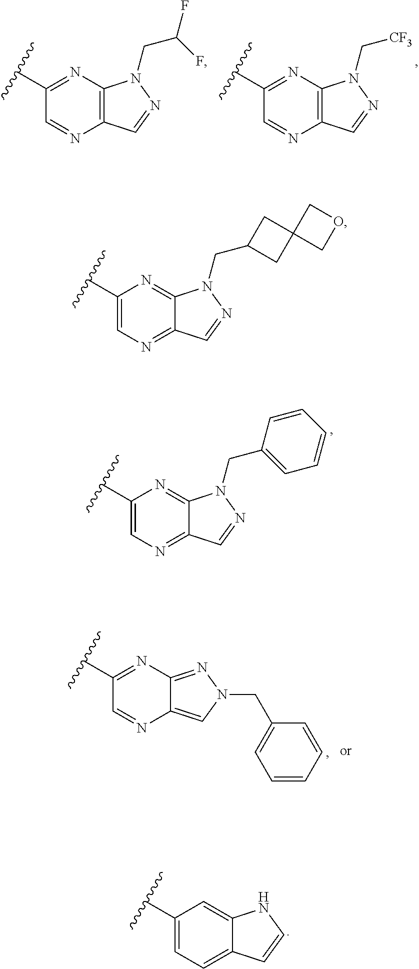

In certain embodiments, A is

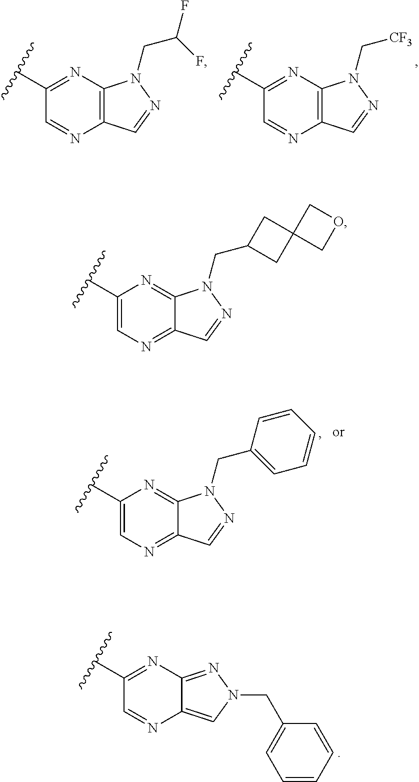

In certain embodiments, A is

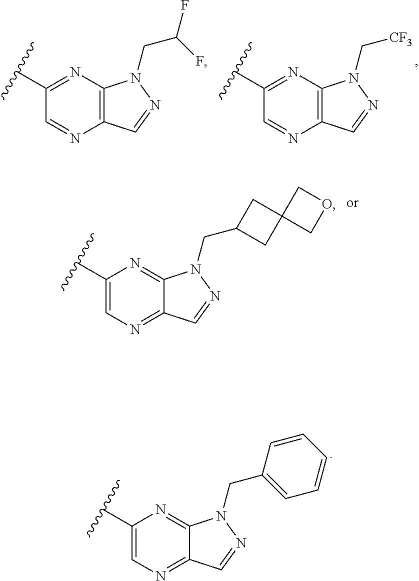

In certain embodiments, A is

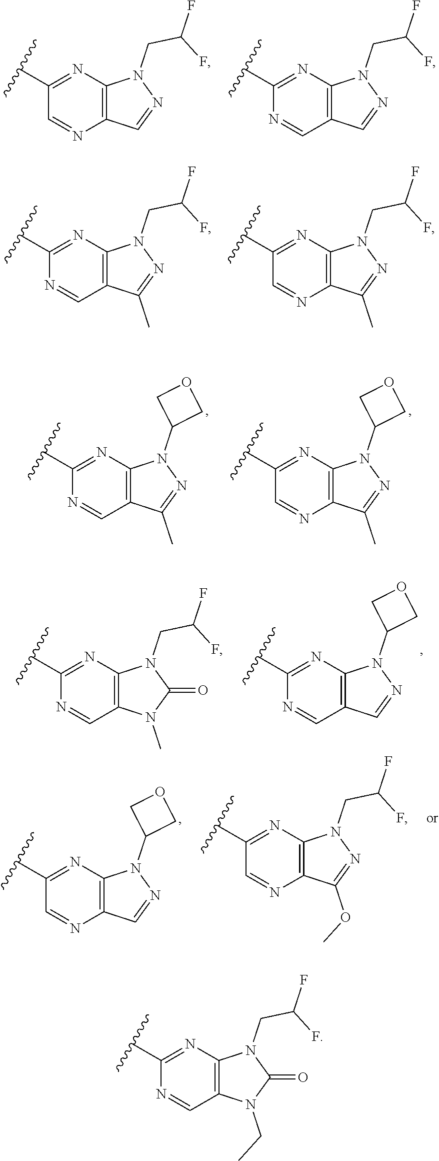

In certain embodiments, A is

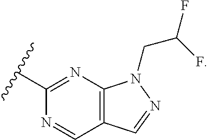 [SMILES: *c1ncc2cnn(CC(F)F)c2n1]

In certain embodiments, A is

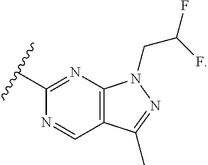 [SMILES: *c1ncc2c(C)nn(CC(F)F)c2n1]

In certain embodiments, A is

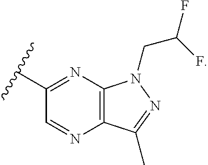 [SMILES: *c1cnc2c(C)nn(CC(F)F)c2n1]

In certain embodiments, A is

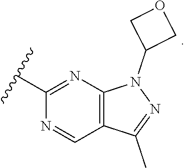 [SMILES: *c1ncc2c(C)nn(C3COC3)c2n1]

In certain embodiments, A is

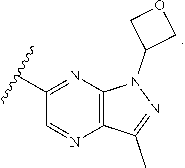 [SMILES: *c1cnc2c(C)nn(C3COC3)c2n1]

In certain embodiments, A is

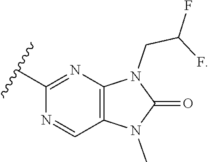 [SMILES: *c1ncc2c(n1)n(CC(F)F)c(=O)n2C]

In certain embodiments, A is

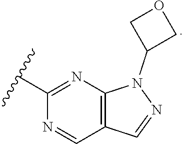 [SMILES: *c1ncc2cnn(C3COC3)c2n1]

In certain embodiments, A is

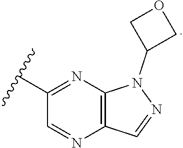 [SMILES: *c1cnc2cnn(C3COC3)c2n1]

In certain embodiments, A is

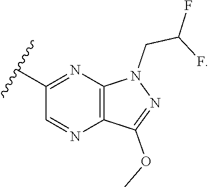 [SMILES: *c1cnc2c(OC)nn(CC(F)F)c2n1]

In certain embodiments, A is

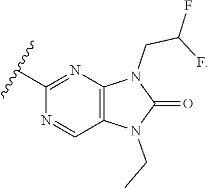 [SMILES: *c1ncc2c(n1)n(CC(F)F)c(=O)n2CC]

In certain embodiments, A is

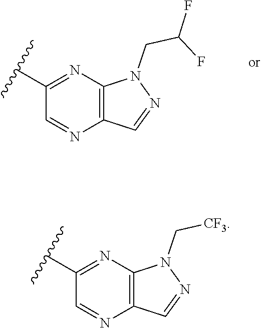 [SMILES: *c1cnc2cnn(CC(F)F)c2n1 ; *c1cnc2cnn(CC(F)(F)F)c2n1]

In certain embodiments, A is

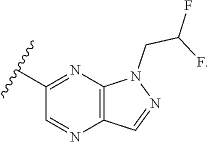 [SMILES: *c1cnc2cnn(CC(F)F)c2n1]

In certain embodiments, A is

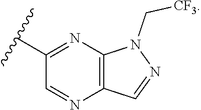 [SMILES: *c1cnc2cnn(CC(F)(F)F)c2n1]

In certain embodiments, A is

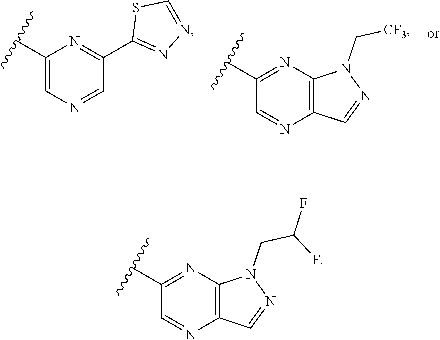 [SMILES: *c1cncc(-c2nncs2)n1 ; *c1cnc2cnn(CC(F)(F)F)c2n1 ; *c1cnc2cnn(CC(F)F)c2n1]

In certain embodiments, A is

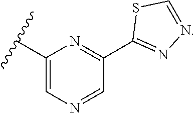 [SMILES: *c1cncc(-c2nncs2)n1]

In certain embodiments, A is

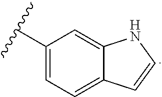 [SMILES: *c1ccc2cc[nH]c2c1]

### CERTAIN EMBODIMENTS

In certain embodiments, the compound of Formula (I) is of Formula (I-a):

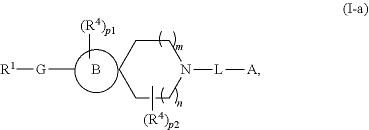

or a pharmaceutically acceptable salt, co-crystal, tautomer, stereoisomer, solvate, hydrate, polymorph, isotopically enriched derivative, or prodrug thereof; wherein R¹, G, R⁴, p1, p2, m, n, L, and A are as defined herein.

In certain embodiments, the compound of Formula (I) is of Formula (I-b):

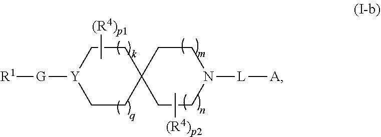

or a pharmaceutically acceptable salt, co-crystal, tautomer, stereoisomer, solvate, hydrate, polymorph, isotopically enriched derivative, or prodrug thereof; wherein R¹, G, R⁴, p1, p2, m, n, L, and A are as defined herein; Y is N or CR⁵; R⁵ is hydrogen, halogen, or substituted or unsubstituted alkyl; and k and q are each independently 0, 1, or 2.

In certain embodiments, Y is N. In certain embodiments, Y is CR⁵. In certain embodiments, Y is CH.

In certain embodiments, m is 0, 1, or 2; n is 0, 1, or 2; k is 0, 1, or 2; and q is 0, 1, or 2. In certain embodiments, m is 0. In certain embodiments, m is 1. In certain embodiments, m is 2. In certain embodiments, n is 0. In certain embodiments, n is 1. In certain embodiments, n is 2. In certain embodiments, k is 0. In certain embodiments, k is 1. In certain embodiments, k is 2. In certain embodiments, q is 0. In certain embodiments, q is 1. In certain embodiments, q is 2.

In certain embodiments, m is 0; and n is 0. In certain embodiments, m is 0; and n is 1. In certain embodiments, m is 0; and n is 2. In certain embodiments, m is 1; and n is 0. In certain embodiments, m is 1; and n is 1. In certain embodiments, m is 1; and n is 2. In certain embodiments, m is 2; and n is 0. In certain embodiments, m is 2; and n is 1. In certain embodiments, m is 2; and n is 2.

In certain embodiments, k is 0; and q is 0. In certain embodiments, k is 0; and q is 1. In certain embodiments, k is 0; and q is 2. In certain embodiments, k is 1; and q is 0. In certain embodiments, k is 1; and q is 1. In certain embodiments, k is 1; and q is 2. In certain embodiments, k is 2; and q is 0. In certain embodiments, k is 2; and q is 1. In certain embodiments, k is 2; and q is 2.

In certain embodiments, m is 0; n is 0; k is 0; and q is 0. In certain embodiments, m is 0; n is 0; k is 1; and q is 0. In certain embodiments, m is 0; n is 0; k is 0; and q is 1. In certain embodiments, m is 0; n is 0; k is 1; and q is 1. In certain embodiments, m is 0; n is 0; k is 2; and q is 0. In certain embodiments, m is 0; n is 0; k is 0; and q is 2.

In certain embodiments, m is 0; n is 1; k is 0; and q is 0. In certain embodiments, m is 0; n is 1; k is 1; and q is 0. In certain embodiments, m is 0; n is 1; k is 0; and q is 1. In certain embodiments, m is 0; n is 1; k is 1; and q is 1. In certain embodiments, m is 0; n is 1; k is 0; and q is 2. In certain embodiments, m is 0; n is 1; k is 2; and q is 0.

In certain embodiments, m is 1; n is 0; k is 0; and q is 0. In certain embodiments, m is 1; n is 0; k is 1; and q is 0. In certain embodiments, m is 1; n is 0; k is 0; and q is 1. In certain embodiments, m is 1; n is 0; k is 1; and q is 1. In certain embodiments, m is 1; n is 0; k is 0; and q is 2. In certain embodiments, m is 1; n is 0; k is 2; and q is 0.

In certain embodiments, m is 1; n is 1; k is 0; and q is 0. In certain embodiments, m is 1; n is 1; k is 1; and q is 0. In certain embodiments, m is 1; n is 1; k is 0; and q is 1. In certain embodiments, m is 1; n is 1; k is 1; and q is 1. In certain embodiments, m is 1; n is 1; k is 0; and q is 2. In certain embodiments, m is 1; n is 1; k is 2; and q is 0.

In certain embodiments, m is 0; n is 2; k is 0; and q is 0. In certain embodiments, m is 0; n is 2; k is 1; and q is 0. In certain embodiments, m is 0; n is 2; k is 0; and q is 1. In certain embodiments, m is 0; n is 2; k is 1; and q is 1. In certain embodiments, m is 0; n is 2; k is 0; and q is 2. In certain embodiments, m is 0; n is 2; k is 2; and q is 0.

In certain embodiments, m is 2; n is 0; k is 0; and q is 0. In certain embodiments, m is 2; n is 0; k is 1; and q is 0. In certain embodiments, m is 2; n is 0; k is 0; and q is 1. In certain embodiments, m is 2; n is 0; k is 1; and q is 1. In certain embodiments, m is 2; n is 0; k is 0; and q is 2. In certain embodiments, m is 2; n is 0; k is 2; and q is 0.

In certain embodiments, the compound of Formula (I) is of Formula (I-c):

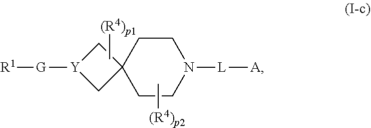

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-c) is of Formula (I-c-1):

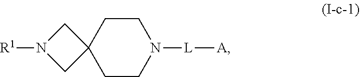

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-c) is of Formula (I-c-2):

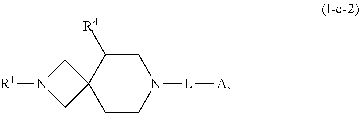

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-c) is of Formula (I-c-3):

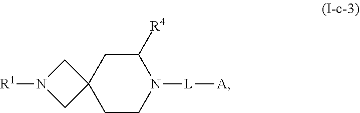

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I-c) is of Formula (I-c-4):

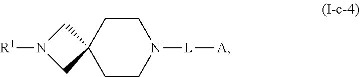

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-c) is of Formula (I-c-5):

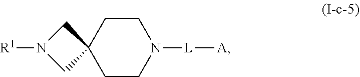

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-d):

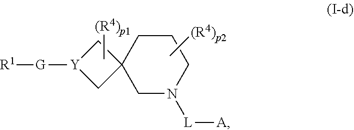

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-d) is of Formula (I-d-1):

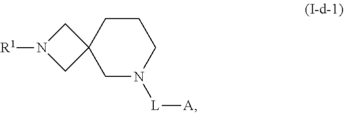

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-d) is of Formula (I-d-2):

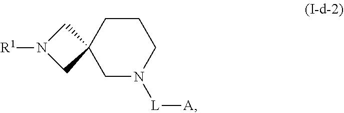

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-d) is of Formula (I-d-3):

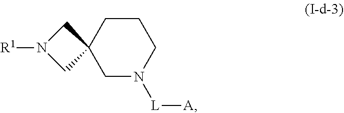

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-d) is of Formula (I-d-4):

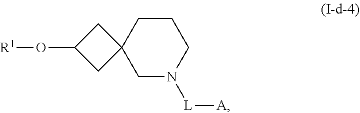

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-d) is of Formula (I-d-5):

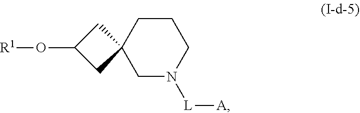

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-d) is of Formula (I-d-6):

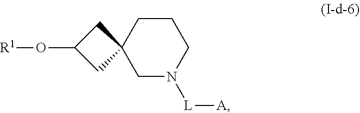

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-e):

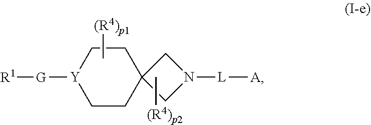

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-e) is of Formula (I-e-1):

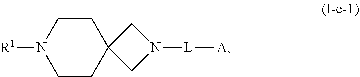

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-e) is of Formula (I-e-2):

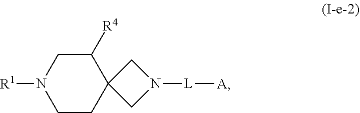

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-e) is of Formula (I-e-3):

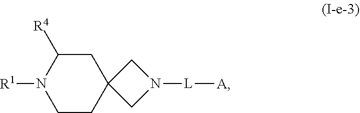

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I-e) is of Formula (I-e-4):

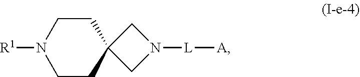

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-e) is of Formula (I-e-5):

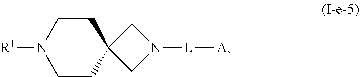

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-f):

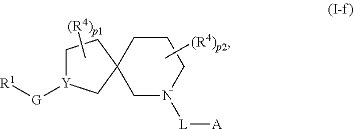

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-f) is of Formula (I-f-1):

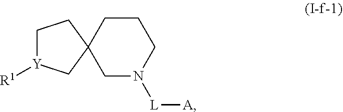

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-f) is of Formula (I-f-2):

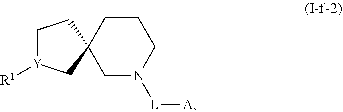

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-f) is of Formula (I-f-3):

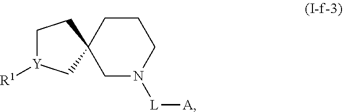

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-f) is of Formula (I-f-4):

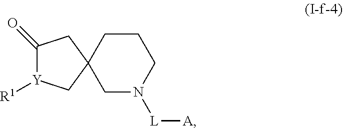

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-f) is of Formula (I-f-5):

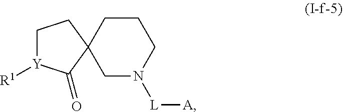

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-g):

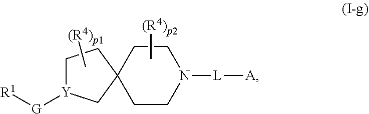

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-g) is of Formula (I-g-1):

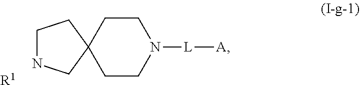

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-g) is of Formula (I-g-2):

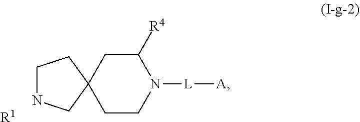

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I-g) is of Formula (I-g-3):

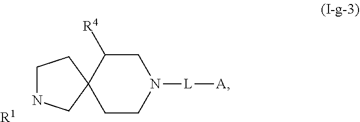

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-g) is of Formula (I-g-4):

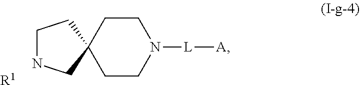

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-g) is of Formula (I-g-5):

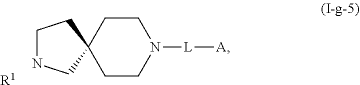

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-g) is of Formula (I-g-6):

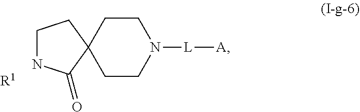

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-g) is of Formula (I-g-7):

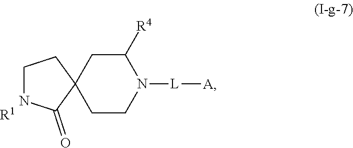

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I-g) is of Formula (I-g-8):

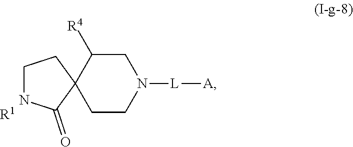

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-g) is of Formula (I-g-9):

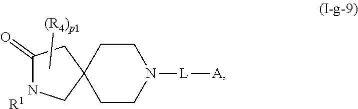

or a pharmaceutically acceptable salt, co-crystal, tautomer, stereoisomer, solvate, hydrate, polymorph, isotopically enriched derivative, or prodrug thereof; wherein R¹, R⁴, p1, L, and A are as defined herein.

In certain embodiments, the compound of Formula (I-g) is of Formula (I-g-10):

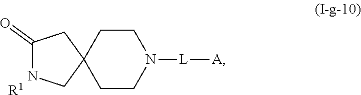

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-g) is of Formula (I-g-11):

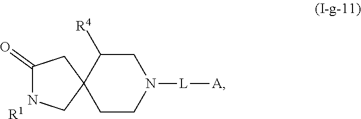

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-g) is of Formula (I-g-12):

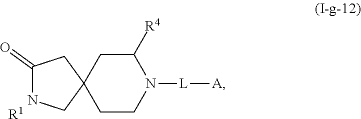

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I-g) is of Formula (I-g-13):

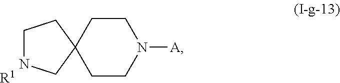

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I) is of Formula (I-h):

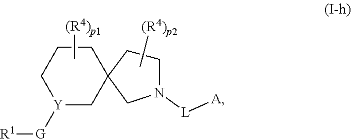

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-h) is of Formula (I-h-1):

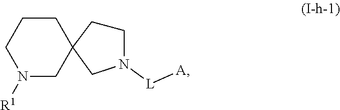

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-h) is of Formula (I-h-2):

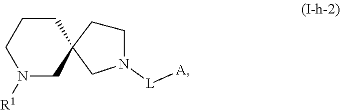

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-h) is of Formula (I-h-3):

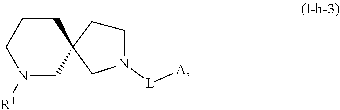

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-i):

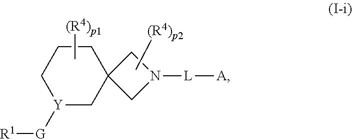

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-i) is of Formula (I-i-1):

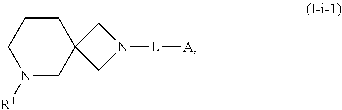

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-i) is of Formula (I-i-2):

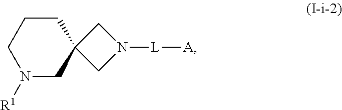

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-i) is of Formula (I-i-3):

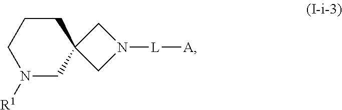

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I) is of Formula (I-j):

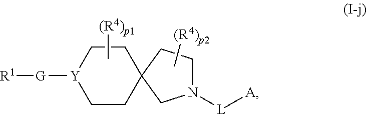

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-j) is of Formula (I-j-1):

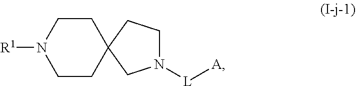

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-j) is of Formula (I-j-2):

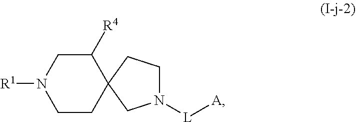

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-j) is of Formula (I-j-3):

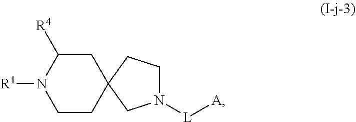

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I-j) is of Formula (I-j-4):

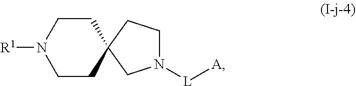

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-j) is of Formula (I-j-5):

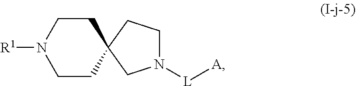

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-j) is of Formula (I-j-6):

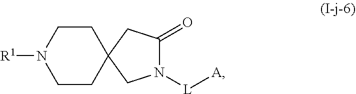

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-j) is of Formula (I-j-7):

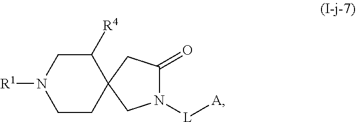

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-j) is of Formula (I-j-8):

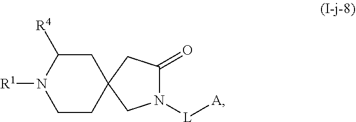

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I-j) is of Formula (I-j-9):

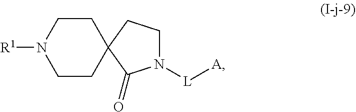

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-j) is of Formula (I-j-10):

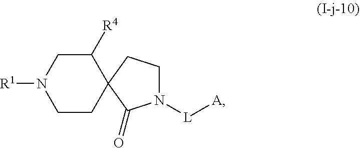

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I-j) is of Formula (I-j-11):

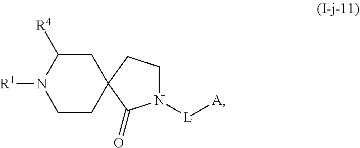

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I) is of Formula (I-k):

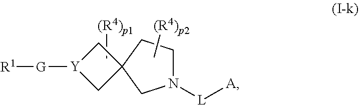

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-k) is of Formula (I-k-1):

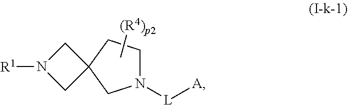

or a pharmaceutically acceptable salt, co-crystal, tautomer, stereoisomer, solvate, hydrate, polymorph, isotopically enriched derivative, or prodrug thereof; wherein R¹, R⁴, p2, L, and A are as defined herein.

In certain embodiments, the compound of Formula (I-k) is of Formula (I-k-2):

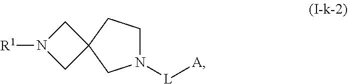

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-k) is of Formula (I-k-3):

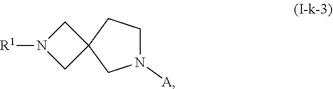

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I-k) is of Formula (I-k-4):

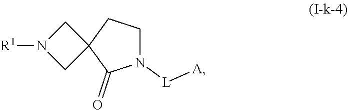

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-k) is of Formula (I-k-5):

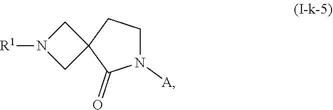

or a pharmaceutically acceptable salt, co-crystal, tautomer, stereoisomer, solvate, hydrate, polymorph, isotopically enriched derivative, or prodrug thereof; wherein R¹ and A are as defined herein. In certain embodiments, the compound of Formula (I) is of Formula (I-l):

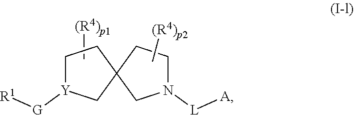

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-l) is of Formula (I-l-1):

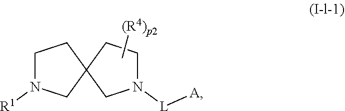

or a pharmaceutically acceptable salt, co-crystal, tautomer, stereoisomer, solvate, hydrate, polymorph, isotopically enriched derivative, or prodrug thereof; wherein R¹, R⁴, p2, L, and A are as defined herein.

In certain embodiments, the compound of Formula (I-l) is of Formula (I-l-2):

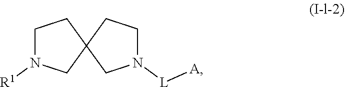

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-l) is of Formula (I-l-3):

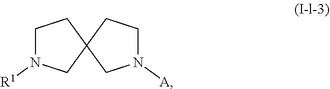

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I-l) is of Formula (I-l-4):

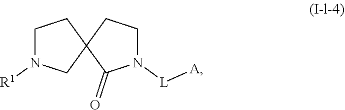

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-l) is of Formula (I-l-5):

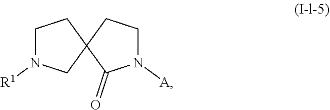

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I) is of Formula (I-m):

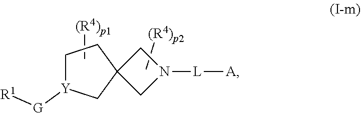

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, L, and A are as defined herein.

In certain embodiments, the compound of Formula (I-m) is of Formula (I-m-1):

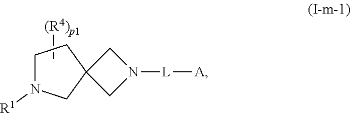

or a pharmaceutically acceptable salt, co-crystal, tautomer, stereoisomer, solvate, hydrate, polymorph, isotopically enriched derivative, or prodrug thereof; wherein R¹, R⁴, p1, L, and A are as defined herein.

In certain embodiments, the compound of Formula (I-m) is of Formula (I-m-2):

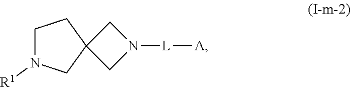

or a pharmaceutically acceptable salt, co-crystal, tautomer, stereoisomer, solvate, hydrate, polymorph, isotopically enriched derivative, or prodrug thereof; wherein R¹, L, and A are as defined herein.

In certain embodiments, the compound of Formula (I-m) is of Formula (I-m-3):

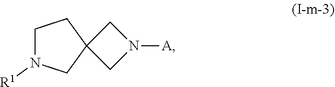

or a pharmaceutically acceptable salt, co-crystal, tautomer, stereoisomer, solvate, hydrate, polymorph, isotopically enriched derivative, or prodrug thereof; wherein R¹ and A are as defined herein.

In certain embodiments, the compound of Formula (I) is of Formula (I-n) or (I-n′):

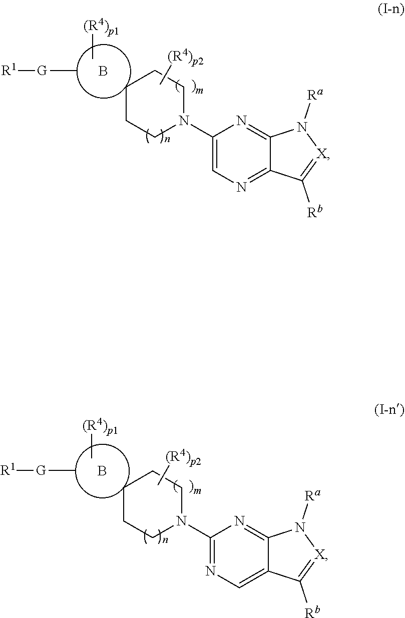

or a pharmaceutically acceptable salt, co-crystal, tautomer, stereoisomer, solvate, hydrate, polymorph, isotopically enriched derivative, or prodrug thereof; wherein R¹, G, B, R⁴, p1, p2, m, and n are as defined herein; X is N or CR^{a}; each R^{a} is independently hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

In certain embodiments, each R^{a} is independently hydrogen, or substituted or unsubstituted alkyl; and R^{b} is halogen, substituted or unsubstituted alkyl, or substituted or unsubstituted alkoxy. In certain embodiments, X is N or CH. In certain embodiments, X is N. In certain embodiments, X is CH. In certain embodiments, X is N or CH; R^{a} is hydrogen or haloalkyl; and R^{b} is halogen, substituted or unsubstituted alkyl, or substituted or unsubstituted alkoxy. In certain embodiments, X is N or CH; R^{a} is hydrogen or fluoroalkyl; and R^{b} is halogen, substituted or unsubstituted alkyl, or substituted or unsubstituted alkoxy. In certain embodiments, X is N or CH; R^{a} is hydrogen or C₁₋₄ fluoroalkyl; and R^{b} is halogen, substituted or unsubstituted alkyl, or substituted or unsubstituted alkoxy. In certain embodiments, X is N; R^{a} is hydrogen or haloalkyl; and R^{b} is halogen, substituted or unsubstituted alkyl, or substituted or unsubstituted alkoxy. In certain embodiments, X is N; R^{a} is hydrogen or fluoroalkyl; and R^{b} is halogen, substituted or unsubstituted alkyl, or substituted or unsubstituted alkoxy. In certain embodiments, X is N; R^{a} is hydrogen or C₁₋₄ fluoroalkyl; and R^{b} is halogen, substituted or unsubstituted alkyl, or substituted or unsubstituted alkoxy. In certain embodiments, X is N; R^{a} is haloalkyl; and R^{b} is halogen, substituted or unsubstituted alkyl, or substituted or unsubstituted alkoxy. In certain embodiments, X is N; R^{a} is fluoroalkyl; and R^{b} is halogen, substituted or unsubstituted alkyl, or substituted or unsubstituted alkoxy. In certain embodiments, X is N; R^{a} is C₁₋₄ fluoroalkyl; and R^{b} is halogen, substituted or unsubstituted alkyl, or substituted or unsubstituted alkoxy. In certain embodiments, X is CH; and R^{a} is hydrogen or substituted or unsubstituted alkyl. In certain embodiments, X is CH; and R^{a} is hydrogen or haloalkyl. In certain embodiments, X is CH; and R^{a} is hydrogen or fluoroalkyl. In certain embodiments, X is CH; and R^{a} is hydrogen or C₁₋₄ fluoroalkyl. In certain embodiments, X is CH; and R^{a} is hydrogen.

In certain embodiments, the compound of Formula (I-n) is of Formula (I-n-1):

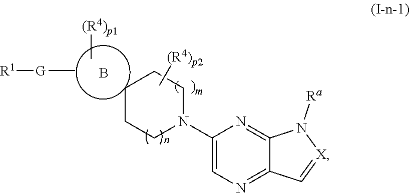

or a pharmaceutically acceptable salt, co-crystal, tautomer, stereoisomer, solvate, hydrate, polymorph, isotopically enriched derivative, or prodrug thereof; wherein R¹, G, B, R⁴, p1, p2, m, and n are as defined herein; X is N or CR^{a}; and each R^{a} is independently hydrogen, substituted or unsubstituted alkyl, or substituted or unsubstituted heterocyclyl.

In certain embodiments, each R^{a} is independently hydrogen, or substituted or unsubstituted alkyl. In certain embodiments, X is N or CH. In certain embodiments, X is N. In certain embodiments, X is CH. In certain embodiments, X is N or CH; and R^{a} is hydrogen or haloalkyl. In certain embodiments, X is N or CH; and R^{a} is hydrogen or fluoroalkyl. In certain embodiments, X is N or CH; and R^{a} is hydrogen or C₁₋₄ fluoroalkyl. In certain embodiments, X is N; and R^{a} is hydrogen or haloalkyl. In certain embodiments, X is N; and R^{a} is hydrogen or fluoroalkyl. In certain embodiments, X is N; and R^{a} is hydrogen or C₁₋₄ fluoroalkyl. In certain embodiments, X is N; and R^{a} is haloalkyl. In certain embodiments, X is N; and R^{a} is fluoroalkyl. In certain embodiments, X is N; and R^{a} is C₁₋₄ fluoroalkyl. In certain embodiments, X is CH; and R^{a} is hydrogen or substituted or unsubstituted alkyl. In certain embodiments, X is CH; and R^{a} is hydrogen or haloalkyl. In certain embodiments, X is CH; and R^{a} is hydrogen or fluoroalkyl. In certain embodiments, X is CH; and R^{a} is hydrogen or C₁₋₄ fluoroalkyl. In certain embodiments, X is CH; and R^{a} is hydrogen.

In certain embodiments, the compound of Formula (I) is of Formula (I-o) or (I-o′):

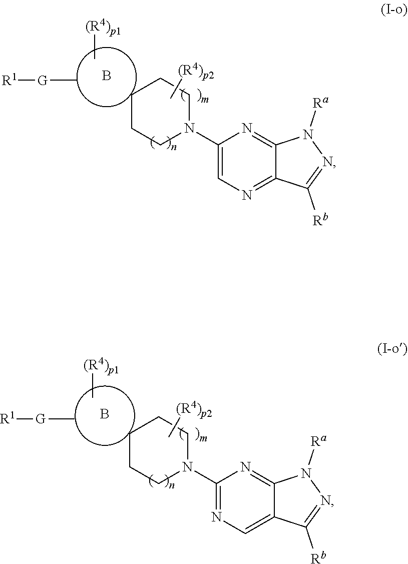

or a pharmaceutically acceptable salt, co-crystal, tautomer, stereoisomer, solvate, hydrate, polymorph, isotopically enriched derivative, or prodrug thereof; wherein R¹, G, B, R⁴, p1, p2, m, and n are as defined herein; R^{a} is hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

In certain embodiments, the compound of Formula (I-o) is of Formula (I-o-l):

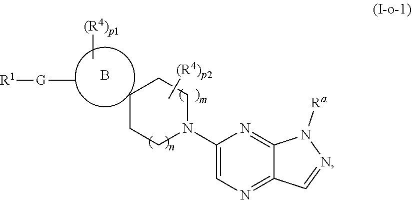

or a pharmaceutically acceptable salt, co-crystal, tautomer, stereoisomer, solvate, hydrate, polymorph, isotopically enriched derivative, or prodrug thereof; wherein R¹, G, B, R⁴, p1, p2, m, and n are as defined herein; and R^{a} is hydrogen, substituted or unsubstituted alkyl, or substituted or unsubstituted heterocyclyl.

In certain embodiments of Formula (I-o), R^{a} is hydrogen, or substituted or unsubstituted alkyl. In certain embodiments, R^{a} is substituted or unsubstituted alkyl or substituted or unsubstituted heterocyclyl. In certain embodiments, R^{a} is substituted or unsubstituted alkyl. In certain embodiments, R^{a} is haloalkyl. In certain embodiments, R^{a} is fluoroalkyl. In certain embodiments, R^{a} is C₁₋₄ fluoroalkyl. In certain embodiments, R^{a} is 2,2-difluoroethyl, or 2,2,2-trifluoroethyl. In certain embodiments, R^{a} is 2,2-difluoroethyl. In certain embodiments, R^{a} is 2,2,2-trifluoroethyl.

In certain embodiments, the compound of Formula (I) is of Formula (I-p) or (I-p′):

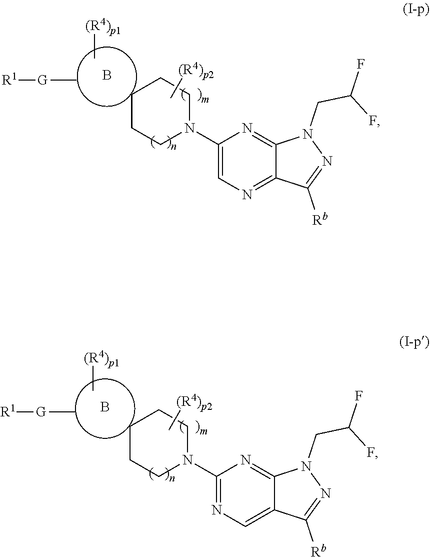

or a pharmaceutically acceptable salt, co-crystal, tautomer, stereoisomer, solvate, hydrate, polymorph, isotopically enriched derivative, or prodrug thereof; wherein R¹, G, B, R⁴, p1, p2, m, and n are as defined herein; and R^{b} is hydrogen, halogen, substituted or unsubstituted alkyl, or substituted or unsubstituted alkoxy.

In certain embodiments, the compound of Formula (I-p) is of Formula (I-p-1):

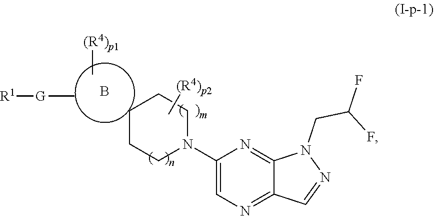

or a pharmaceutically acceptable salt, co-crystal, tautomer, stereoisomer, solvate, hydrate, polymorph, isotopically enriched derivative, or prodrug thereof; wherein R¹, G, B, R⁴, p1, p2, m, and n are as defined herein.

In certain embodiments, the compound of Formula (I) is of Formula (I-q):

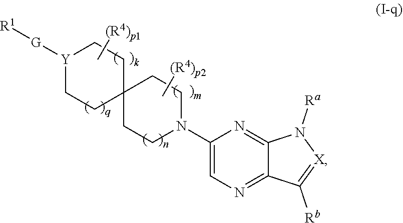

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; each R^{a} is independently hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

In certain embodiments of Formula (I-n), each R^{a} is independently hydrogen, or substituted or unsubstituted alkyl. In certain embodiments, X is N or CH. In certain embodiments, X is N. In certain embodiments, X is CH. In certain embodiments, X is N or CH; and R^{a} is hydrogen or haloalkyl. In certain embodiments, X is N or CH; and R^{a} is hydrogen or fluoroalkyl. In certain embodiments, X is N or CH; and R^{a} is hydrogen or C₁₋₄ fluoroalkyl. In certain embodiments, X is N; and R^{a} is hydrogen or haloalkyl. In certain embodiments, X is N; and R^{a} is hydrogen or fluoroalkyl. In certain embodiments, X is N; and R^{a} is hydrogen or C₁₋₄ fluoroalkyl. In certain embodiments, X is N; and R^{a} is haloalkyl. In certain embodiments, X is N; and R^{a} is fluoroalkyl. In certain embodiments, X is N; and R^{a} is C₁₋₄ fluoroalkyl. In certain embodiments, X is CH; and R^{a} is hydrogen or substituted or unsubstituted alkyl. In certain embodiments, X is CH; and R^{a} is hydrogen or haloalkyl. In certain embodiments, X is CH; and R^{a} is hydrogen or fluoroalkyl. In certain embodiments, X is CH; and R^{a} is hydrogen or C₁₋₄ fluoroalkyl. In certain embodiments, X is CH; and R^{a} is hydrogen.

In certain embodiments, the compound of Formula (I) is of Formula (I-q-1):

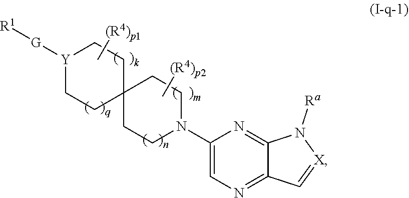

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and each R^{a} is independently hydrogen, substituted or unsubstituted alkyl, or substituted or unsubstituted heterocyclyl.

In certain embodiments, the compound of Formula (I-q) is of Formula (I-q-2):

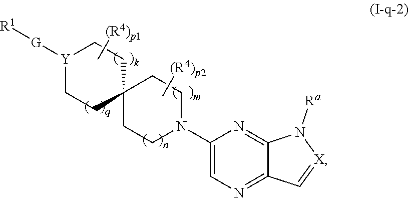

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and each R^{a} is independently hydrogen, substituted or unsubstituted alkyl, or substituted or unsubstituted heterocyclyl.

In certain embodiments, the compound of Formula (I-q) is of Formula (I-q-3):

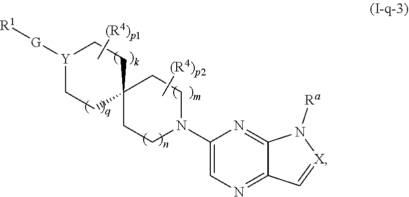

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and each R^{a} is independently hydrogen, substituted or unsubstituted alkyl, or substituted or unsubstituted heterocyclyl.

In certain embodiments, the compound of Formula (I-q) is of Formula (I-q-4):

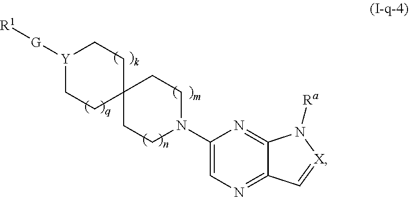

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and each R^{a} is independently hydrogen, substituted or unsubstituted alkyl, or substituted or unsubstituted heterocyclyl.

In certain embodiments, the compound of Formula (I-n) is of Formula (I-q-5):

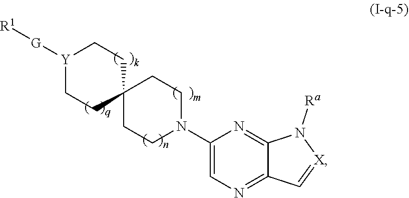

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and each R^{a} is independently hydrogen, substituted or unsubstituted alkyl, or substituted or unsubstituted heterocyclyl.

In certain embodiments, the compound of Formula (I-q) is of Formula (I-q-6):

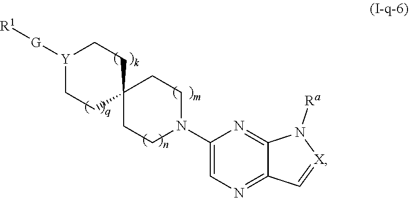

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and each R^{a} is independently hydrogen, substituted or unsubstituted alkyl, or substituted or unsubstituted heterocyclyl.

In certain embodiments, the compound of Formula (I) is of Formula (I-r):

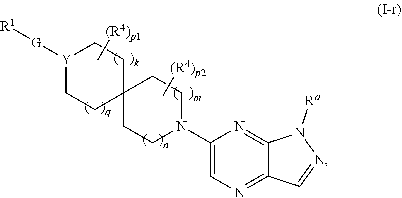

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; R^{a} is hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

In certain embodiments of Formula (I-r), R^{a} is hydrogen, or substituted or unsubstituted alkyl. In certain embodiments, R^{a} is substituted or unsubstituted alkyl or substituted or unsubstituted heterocyclyl. In certain embodiments, R^{a} is substituted or unsubstituted alkyl. In certain embodiments, R^{a} is haloalkyl. In certain embodiments, R^{a} is fluoroalkyl. In certain embodiments, R^{a} is C₁₋₄ fluoroalkyl. In certain embodiments, R^{a} is 2,2-difluoroethyl, or 2,2,2-trifluoroethyl. In certain embodiments, R^{a} is 2,2-difluoroethyl. In certain embodiments, R^{a} is 2,2,2-trifluoroethyl.

In certain embodiments, the compound of Formula (I) is of Formula (I-r-1):

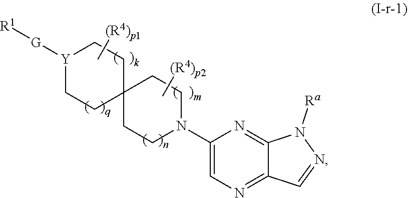

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; and R^{a} is hydrogen, substituted or unsubstituted alkyl, or substituted or unsubstituted heterocyclyl.

In certain embodiments, the compound of Formula (I-r) is of Formula (I-r-2):

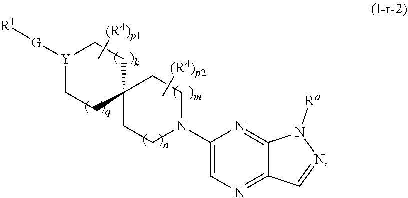

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; and R^{a} is hydrogen, substituted or unsubstituted alkyl, or substituted or unsubstituted heterocyclyl.

In certain embodiments, the compound of Formula (I-r) is of Formula (I-r-3):

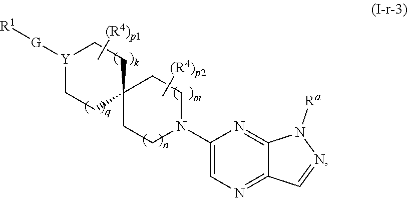

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and R^{a} is hydrogen, substituted or unsubstituted alkyl, or substituted or unsubstituted heterocyclyl.

In certain embodiments, the compound of Formula (I-r) is of Formula (I-r-4):

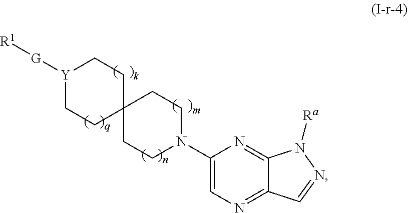

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and R^{a} is hydrogen, substituted or unsubstituted alkyl, or substituted or unsubstituted heterocyclyl.

In certain embodiments, the compound of Formula (I-r) is of Formula (I-r-5):

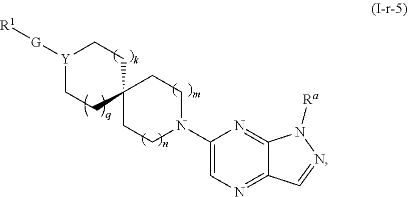

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and R^{a} is hydrogen, substituted or unsubstituted alkyl, or substituted or unsubstituted heterocyclyl.

In certain embodiments, the compound of Formula (I-r) is of Formula (I-r-6):

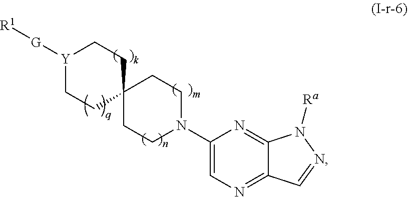

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; X is N or CR^{a}; and R^{a} is hydrogen, substituted or unsubstituted alkyl, or substituted or unsubstituted heterocyclyl.

In certain embodiments, the compound of Formula (I) is of Formula (I-s):

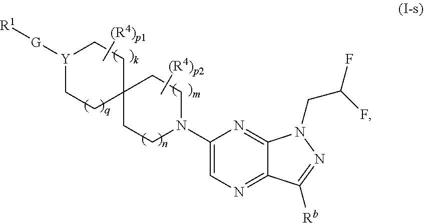

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein; and R^{b} is hydrogen, halogen, substituted or unsubstituted alkyl, or substituted or unsubstituted alkoxy.

In certain embodiments, the compound of Formula (I-s) is of Formula (I-s-1):

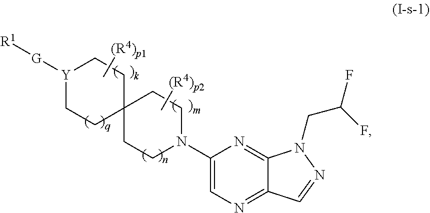

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-s) is of Formula (I-s-2):

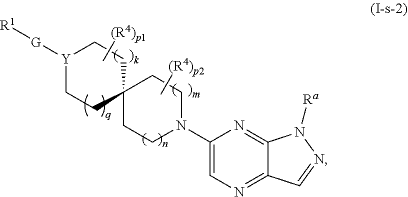

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-s) is of Formula (I-s-3):

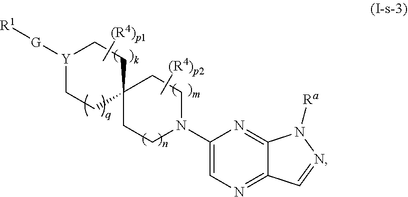

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-s) is of Formula (I-s-4):

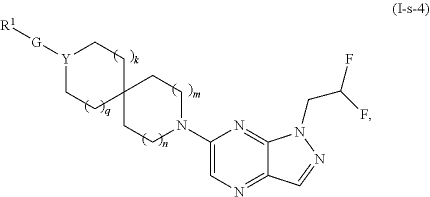

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-s) is of Formula (I-s-5):

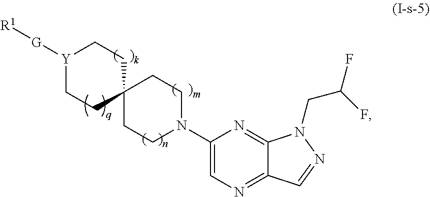

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-s) is of Formula (I-s-6):

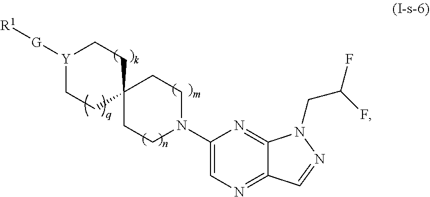

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I) is of Formula (I-t):

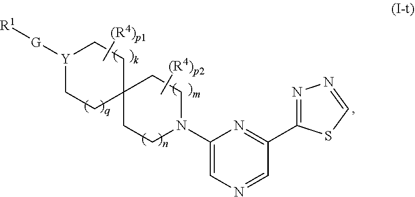

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-t) is of Formula (I-t-1):

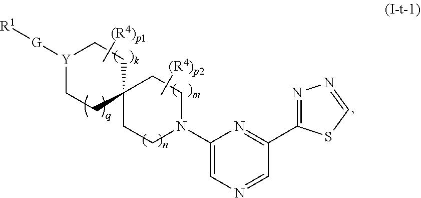

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-t) is of Formula (I-t-2):

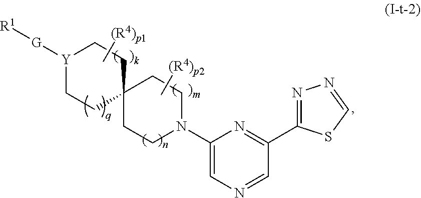

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-t) is of Formula (I-t-3):

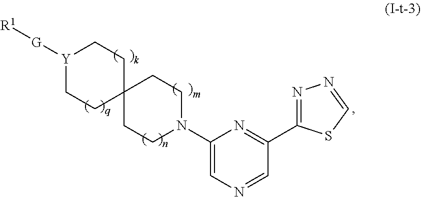

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-t) is of Formula (I-t-4):

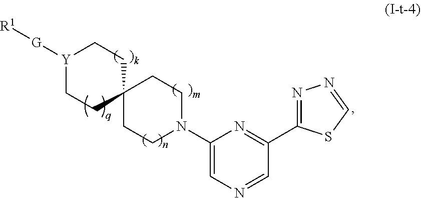

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-t) is of Formula (I-t-5):

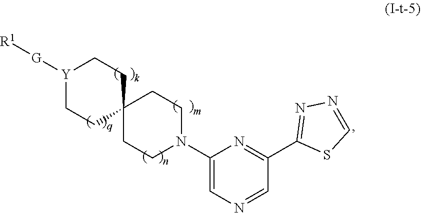

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I) is of Formula (I-u):

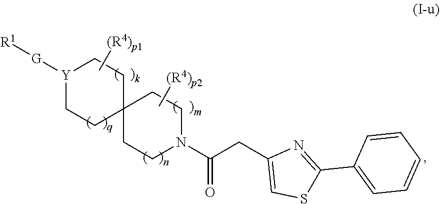

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-r) is of Formula (I-u-1):

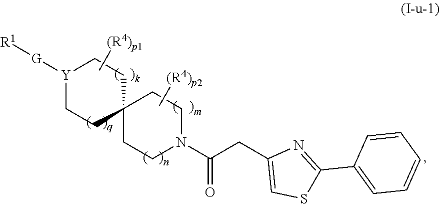

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-r) is of Formula (I-u-2):

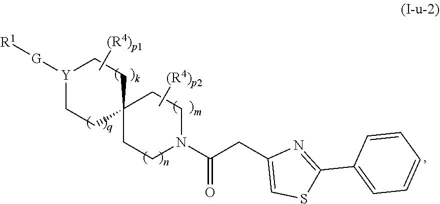

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-u) is of Formula (I-u-3):

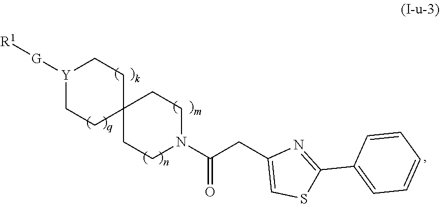

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-r) is of Formula (I-u-4):

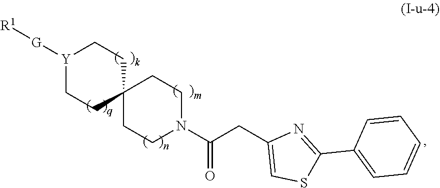

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I-u) is of Formula (I-u-5):

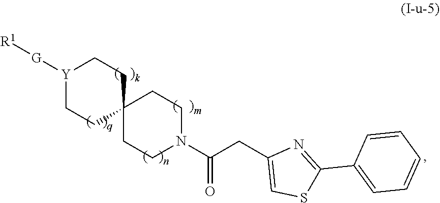

or a pharmaceutically acceptable salt, co-crystal, tautomer, stereoisomer, solvate, hydrate, polymorph, isotopically enriched derivative, or prodrug thereof; wherein R¹, G, Y, R⁴, p1, p2, m, n, k, and q are as defined herein.

In certain embodiments, the compound of Formula (I) is of Formula (I-v):

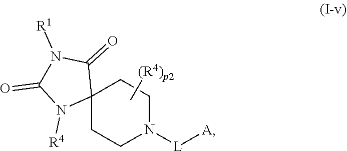

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I) is of Formula (I-v-1):

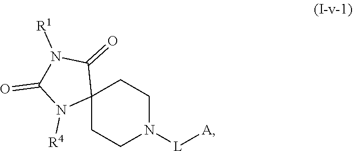

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, the compound of Formula (I) is of Formula (I-v-2):

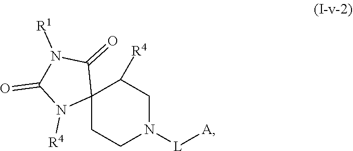

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl or substituted or unsubstituted alkoxy. In certain embodiments, R⁴ is substituted or unsubstituted C₁₋₄ alkyl or substituted or unsubstituted C₁₋₄ alkoxy. In certain embodiments, R⁴ is unsubstituted C₁₋₄ alkyl, C₁₋₄ fluoroalkyl, unsubstituted C₁₋₄ alkoxy, or C₁₋₄ fluoroalkoxy.

In certain embodiments, the compound of Formula (I) is of Formula (I-v-3):

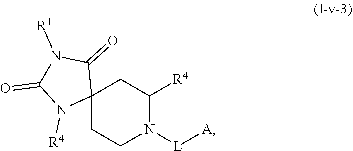

or a pharmaceutically acceptable salt, co-crystal, tautomer, stereoisomer, solvate, hydrate, polymorph, isotopically enriched derivative, or prodrug thereof; wherein R¹, R⁴, L, and A are as defined herein.

In certain embodiments, R⁴ is substituted or unsubstituted alkyl. In certain embodiments, R⁴ is substituted or unsubstituted C₁₋₄ alkyl. In certain embodiments, R⁴ is unsubstituted C₁₋₄ alkyl or C₁₋₄ fluoroalkyl.

In certain embodiments, the compound of Formula (I) is of Formula (I-v-4):

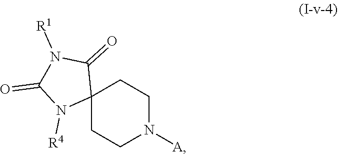

or a pharmaceutically acceptable salt, co-crystal, tautomer, stereoisomer, solvate, hydrate, polymorph, isotopically enriched derivative, or prodrug thereof; wherein R¹, R⁴, and A are as defined herein.

In certain embodiments, the compound of Formula (I) is of Formula (I-w):

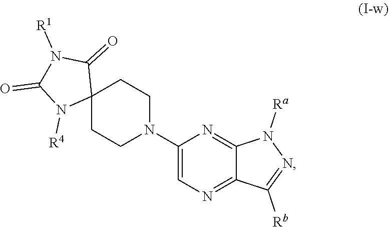

or a pharmaceutically acceptable salt, co-crystal, tautomer, stereoisomer, solvate, hydrate, polymorph, isotopically enriched derivative, or prodrug thereof; wherein R¹, R⁴, and A are as defined herein; R^{a} is hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

In certain embodiments of Formula (I-w), R^{a} is hydrogen, or substituted or unsubstituted alkyl. In certain embodiments, R^{a} is substituted or unsubstituted alkyl or substituted or unsubstituted heterocyclyl. In certain embodiments, R^{a} is substituted or unsubstituted alkyl. In certain embodiments, R^{a} is haloalkyl. In certain embodiments, R^{a} is fluoroalkyl. In certain embodiments, R^{a} is C₁₋₄ fluoroalkyl. In certain embodiments, R^{a} is 2,2-difluoroethyl, or 2,2,2-trifluoroethyl. In certain embodiments, R^{a} is 2,2-difluoroethyl. In certain embodiments, R^{a} is 2,2,2-trifluoroethyl.

In certain embodiments, the compound of Formula (I-x) is of Formula (I-w-1):

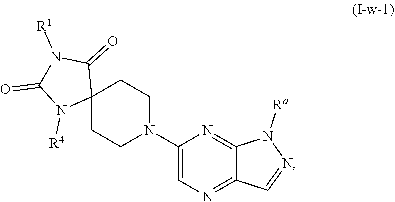

or a pharmaceutically acceptable salt, co-crystal, tautomer, stereoisomer, solvate, hydrate, polymorph, isotopically enriched derivative, or prodrug thereof; wherein R¹, R⁴, and A are as defined herein; and R^{a} is hydrogen, substituted or unsubstituted alkyl, or substituted or unsubstituted heterocyclyl.

In certain embodiments, the compound of Formula (I) is of Formula (I-w-2):

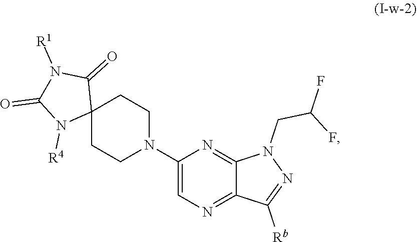

or a pharmaceutically acceptable salt, co-crystal, tautomer, stereoisomer, solvate, hydrate, polymorph, isotopically enriched derivative, or prodrug thereof; wherein R¹ and R⁴ are as defined herein; and R^{b} is hydrogen, halogen, substituted or unsubstituted alkyl, or substituted or unsubstituted alkoxy.

In certain embodiments, the compound of Formula (I-w) is of Formula (I-w-3):

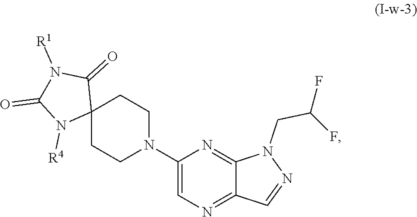

or a pharmaceutically acceptable salt, co-crystal, tautomer, stereoisomer, solvate, hydrate, polymorph, isotopically enriched derivative, or prodrug thereof; wherein R¹ and R⁴ are as defined herein.

In certain embodiments, the compound of Formula (I) is of Formula (I-x):

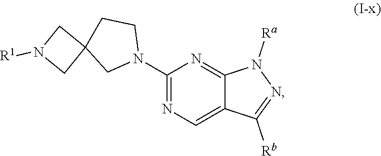

or a pharmaceutically acceptable salt, co-crystal, tautomer, stereoisomer, solvate, hydrate, polymorph, isotopically enriched derivative, or prodrug thereof; wherein R¹ is as defined herein; R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is C₁₋₄ haloalkyl or unsubstituted 4-5 membered heterocyclyl; and R^{b} is hydrogen or unsubstituted C₁₋₄ alkyl. In certain embodiments, R^{a} is 2,2-difluoroethyl or oxetanyl; and R^{b} is hydrogen, —CH₃, or —OCH₃. In certain embodiments, R^{a} is 2,2-difluoroethyl or oxetanyl; and R^{b} is hydrogen or —CH₃. In certain embodiments, R^{a} is 2,2-difluoroethyl; and R^{b} is hydrogen.

In certain embodiments, the compound of Formula (I-x) is of Formula (I-x-1):

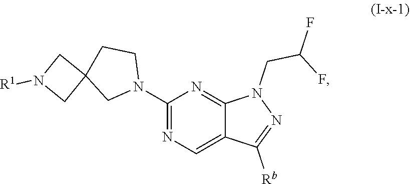

or a pharmaceutically acceptable salt, co-crystal, tautomer, stereoisomer, solvate, hydrate, polymorph, isotopically enriched derivative, or prodrug thereof; wherein R¹ is as defined herein; and R^{b} is hydrogen, substituted or unsubstituted alkyl, or substituted or unsubstituted alkoxy. In certain embodiments, R^{b} is hydrogen, unsubstituted alkyl, or unsubstituted alkoxy. In certain embodiments, R^{b} is hydrogen, unsubstituted alkyl, or unsubstituted alkoxy. In certain embodiments, R^{b} is hydrogen or unsubstituted alkoxy. In certain embodiments, R^{b} is hydrogen, unsubstituted C₁₋₄ alkyl, or unsubstituted C₁₋₄ alkoxy. In certain embodiments, R^{b} is hydrogen or unsubstituted C₁₋₄ alkyl. In certain embodiments, R^{b} is hydrogen, —CH₃, or —OCH₃. In certain embodiments, R^{b} is hydrogen or —CH₃. In certain embodiments, R^{b} is hydrogen.

In certain embodiments, the compound of Formula (I-x) is of Formula (I-x-2):

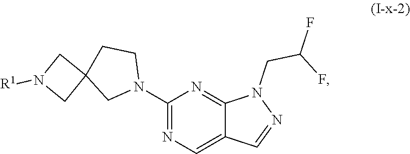

or a pharmaceutically acceptable salt, co-crystal, tautomer, stereoisomer, solvate, hydrate, polymorph, isotopically enriched derivative, or prodrug thereof; wherein R¹ is as defined herein.

In certain embodiments, the compound of Formula (I) is of Formula (I-y):

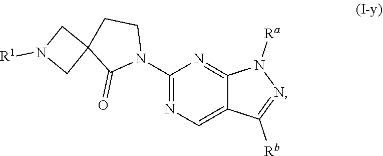

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is C₁₋₄ haloalkyl or unsubstituted 4-5 membered heterocyclyl; and R^{b} is hydrogen or unsubstituted C₁₋₄ alkyl. In certain embodiments, R^{a} is 2,2-difluoroethyl or oxetanyl; and R^{b} is hydrogen, —CH₃, or —OCH₃. In certain embodiments, R^{a} is 2,2-difluoroethyl or oxetanyl; and R^{b} is hydrogen or —CH₃. In certain embodiments, R^{a} is 2,2-difluoroethyl; and R^{b} is hydrogen.

In certain embodiments, the compound of Formula (I) is of Formula (I-z):

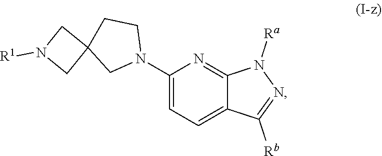

or a pharmaceutically acceptable salt, co-crystal, tautomer, stereoisomer, solvate, hydrate, polymorph, isotopically enriched derivative, or prodrug thereof; wherein R¹ is as defined herein; R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, haloalkoxyalkyl, heterocyclyalkyl, arylalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is 2,2-difluoroethyl or oxetanyl; and R^{b} is hydrogen, —CH₃, or —OCH₃. In certain embodiments, R^{a} is 2,2-difluoroethyl; and R^{b} is hydrogen.

In certain embodiments, the compound of Formula (I) is of Formula (I-aa):

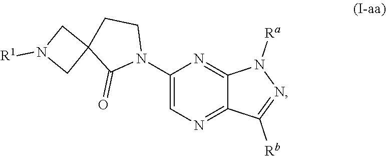

or a pharmaceutically acceptable salt, co-crystal, tautomer, stereoisomer, solvate, hydrate, polymorph, isotopically enriched derivative, or prodrug thereof; wherein R¹ is as defined herein; R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, haloalkoxyalkyl, heterocyclyalkyl, arylalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is 2,2-difluoroethyl or oxetanyl; and R^{b} is hydrogen, —CH₃, or —OCH₃. In certain embodiments, R^{a} is 2,2-difluoroethyl; and R^{b} is hydrogen.

In certain embodiments, the compound of Formula (I) is of Formula (I-bb):

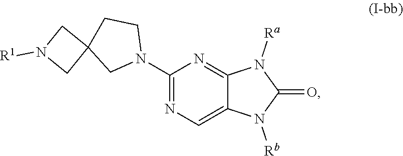

or a pharmaceutically acceptable salt, co-crystal, tautomer, stereoisomer, solvate, hydrate, polymorph, isotopically enriched derivative, or prodrug thereof; wherein R¹ is as defined herein; R^{a} is substituted or unsubstituted alkyl or substituted or unsubstituted heterocyclyl; and R^{b} is substituted or unsubstituted alkyl or substituted or unsubstituted heterocyclyl. In certain embodiments, R^{a} is substituted or unsubstituted alkyl or substituted or unsubstituted heterocyclyl; and R^{b} is substituted or unsubstituted alkyl. In certain embodiments, R^{a} is substituted or unsubstituted alkyl; and R^{b} is substituted or unsubstituted alkyl. In certain embodiments, R^{a} is haloalkyl or heterocyclyl; and R^{b} is unsubstituted alkyl. In certain embodiments, R^{a} is haloalkyl; and R^{b} is unsubstituted alkyl. In certain embodiments, R^{a} is C₁₋₄ haloalkyl or unsubstituted 4-5 membered heterocyclyl; and R^{b} is unsubstituted C₁₋₄ alkyl. In certain embodiments, R^{a} is C₁₋₄ haloalkyl; and R^{b} is unsubstituted C₁₋₄ alkyl. In certain embodiments, R^{a} is 2,2-difluoroethyl or oxetanyl; and R^{b} is —CH₃, or —CH₂CH₃. In certain embodiments, R^{a} is 2,2-difluoroethyl; and R^{b} is —CH₃ or —CH₂CH₃.

In certain embodiments, the compound of Formula (I) is of Formula (I-cc):

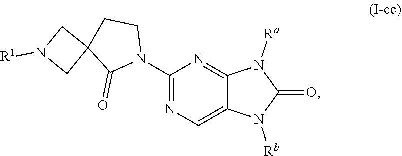

or a pharmaceutically acceptable salt, co-crystal, tautomer, stereoisomer, solvate, hydrate, polymorph, isotopically enriched derivative, or prodrug thereof; wherein R¹ is as defined herein; R^{a} is substituted or unsubstituted alkyl or substituted or unsubstituted heterocyclyl; and R^{b} is substituted or unsubstituted alkyl or substituted or unsubstituted heterocyclyl. In certain embodiments, R^{a} is substituted or unsubstituted alkyl or substituted or unsubstituted heterocyclyl; and R^{b} is substituted or unsubstituted alkyl. In certain embodiments, R^{a} is substituted or unsubstituted alkyl; and R^{b} is substituted or unsubstituted alkyl. In certain embodiments, R^{a} is haloalkyl or heterocyclyl; and R^{b} is unsubstituted alkyl. In certain embodiments, R^{a} is haloalkyl; and R^{b} is unsubstituted alkyl. In certain embodiments, R^{a} is C₁₋₄ haloalkyl or unsubstituted 4-5 membered heterocyclyl; and R^{b} is unsubstituted C₁₋₄ alkyl. In certain embodiments, R^{a} is C₁₋₄ haloalkyl; and R^{b} is unsubstituted C₁₋₄ alkyl. In certain embodiments, R^{a} is 2,2-difluoroethyl or oxetanyl; and R^{b} is —CH₃, or —CH₂CH₃. In certain embodiments, R^{a} is 2,2-difluoroethyl; and R^{b} is —CH₃ or —CH₂CH₃.

In certain embodiments, the compound of Formula (I) is of Formula (I-dd):

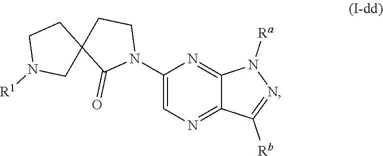

or a pharmaceutically acceptable salt, co-crystal, tautomer, stereoisomer, solvate, hydrate, polymorph, isotopically enriched derivative, or prodrug thereof; wherein R¹ is as defined herein; R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, haloalkoxyalkyl, heterocyclyalkyl, arylalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is 2,2-difluoroethyl or oxetanyl; and R^{b} is hydrogen, —CH₃, or —OCH₃. In certain embodiments, R^{a} is 2,2-difluoroethyl; and R^{b} is hydrogen.

In certain embodiments, the compound of Formula (I) is of Formula (I-ee):

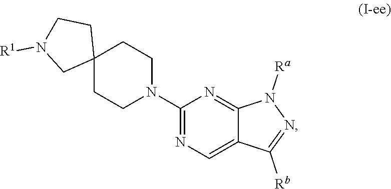

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is C₁₋₄ haloalkyl or unsubstituted 4-5 membered heterocyclyl; and R^{b} is hydrogen or unsubstituted C₁₋₄ alkyl. In certain embodiments, R^{a} is 2,2-difluoroethyl or oxetanyl; and R^{b} is hydrogen, —CH₃, or —OCH₃. In certain embodiments, R^{a} is 2,2-difluoroethyl or oxetanyl; and R^{b} is hydrogen or —CH₃. In certain embodiments, R^{a} is 2,2-difluoroethyl; and R^{b} is hydrogen. In certain embodiments, R^{a} is oxetanyl; and R^{b} is hydrogen.

In certain embodiments, the compound of Formula (I) is of Formula (I-ff):

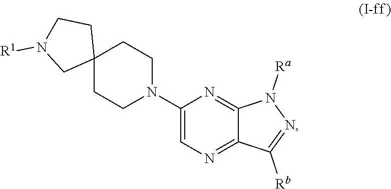

or a pharmaceutically acceptable salt, co-crystal, tautomer, stereoisomer, solvate, hydrate, polymorph, isotopically enriched derivative, or prodrug thereof; wherein R¹ is as defined herein; R^{a} is substituted or unsubstituted alkyl or substituted or unsubstituted heterocyclyl; and R^{b} is hydrogen, substituted or unsubstituted alkyl, or substituted or unsubstituted alkoxy. In certain embodiments, R^{a} is unsubstituted alkyl, haloalkyl, haloalkoxyalkyl, heterocyclyalkyl, arylalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is 2,2-difluoroethyl or oxetanyl; and R^{b} is hydrogen, —CH₃, or —OCH₃. In certain embodiments, R^{a} is 2,2-difluoroethyl; and R^{b} is hydrogen. In certain embodiments, R^{a} is oxetanyl; and R^{b} is —CH₃.

In certain embodiments, the compound of Formula (I) is of Formula (I-gg):

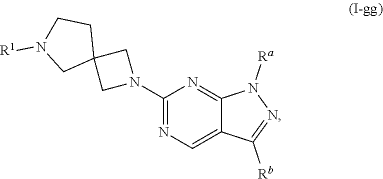

or a pharmaceutically acceptable salt, co-crystal, tautomer, stereoisomer, solvate, hydrate, polymorph, isotopically enriched derivative, or prodrug thereof; wherein R¹ is as defined herein; and R^{a} is substituted or unsubstituted alkyl or substituted or unsubstituted heterocyclyl; R^{b} is hydrogen, substituted or unsubstituted alkyl, or substituted or unsubstituted alkoxy. In certain embodiments, R^{a} is unsubstituted alkyl, haloalkyl, or heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen, unsubstituted alkyl, or unsubstituted alkoxy. In certain embodiments, R^{a} is haloalkyl or unsubstituted heterocyclyl; and R^{b} is hydrogen or unsubstituted alkoxy. In certain embodiments, R^{a} is C₁₋₄ haloalkyl or unsubstituted 4-5 membered heterocyclyl; and R^{b} is hydrogen, unsubstituted C₁₋₄ alkyl, or unsubstituted C₁₋₄ alkoxy. In certain embodiments, R^{a} is C₁₋₄ haloalkyl or unsubstituted 4-5 membered heterocyclyl; and R^{b} is hydrogen or unsubstituted C₁₋₄ alkyl. In certain embodiments, R^{a} is 2,2-difluoroethyl or oxetanyl; and R^{b} is hydrogen, —CH₃, or —OCH₃. In certain embodiments, R^{a} is 2,2-difluoroethyl or oxetanyl; and R^{b} is hydrogen or —CH₃. In certain embodiments, R^{a} is 2,2-difluoroethyl; and R^{b} is hydrogen. In certain embodiments, R^{a} is oxetanyl; and R^{b} is hydrogen. In certain embodiments, R^{a} is 2,2-difluoroethyl; and R^{b} is —CH₃.

In certain embodiments, the compound is of formula (I-g-13), (I-k-3), (I-k-5), (I-l-5), (I-m-3):

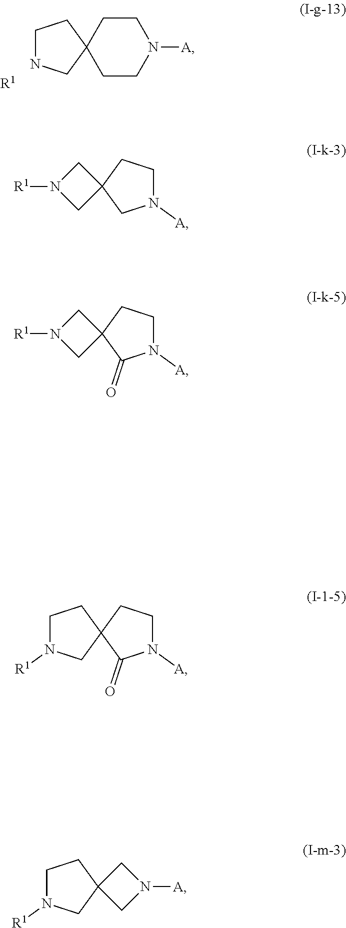

or a pharmaceutically acceptable salt thereof, wherein:
R¹ is

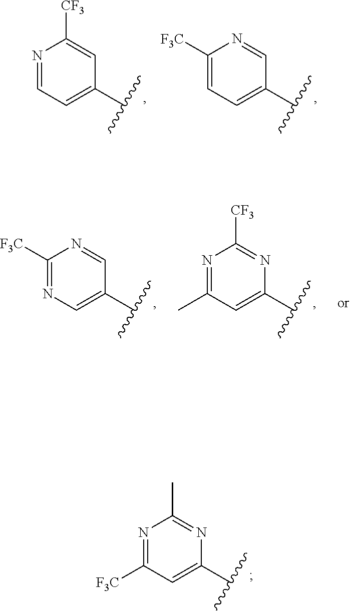

and A is

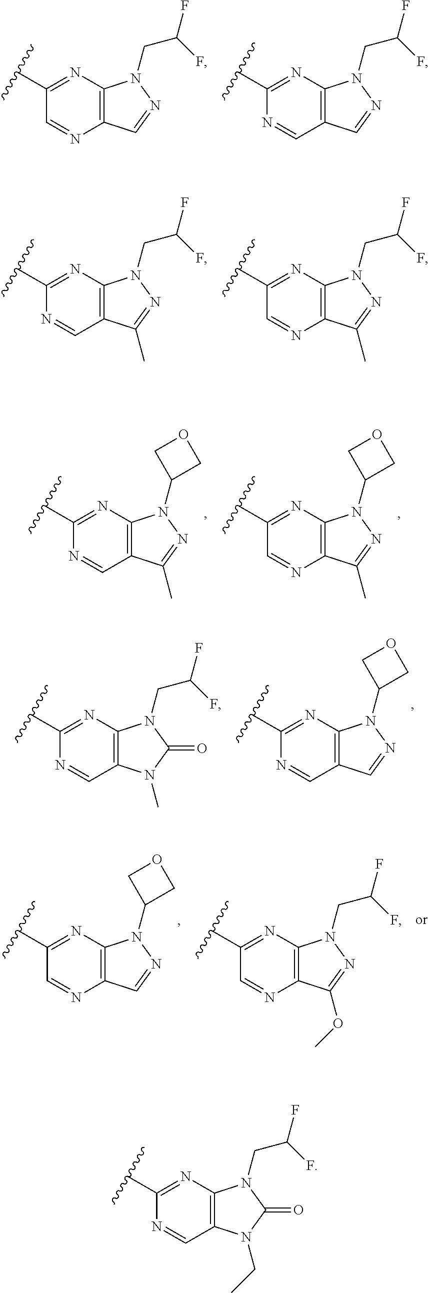

The certain embodiments, the compound of formula (I) is one of the compounds of Table 1, or a pharmaceutically acceptable salt, co-crystal, tautomer, stereoisomer, solvate, hydrate, polymorph, isotopically enriched derivative, or prodrugs thereof:

TABLE 1

| | |
|---|---|
| 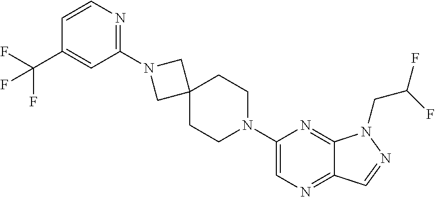 [SMILES: FC(F)Cn1ncc2ncc(N3CCC4(CC3)CN(c3cc(C(F)(F)F)ccn3)C4)nc21] | 1 |
| 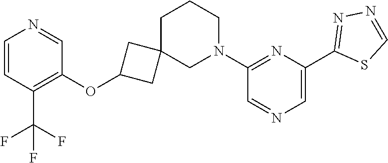 [SMILES: FC(F)(F)c1ccncc1OC1CC2(CCCN(c3cncc(-c4nncs4)n3)C2)C1] | 2 |
| 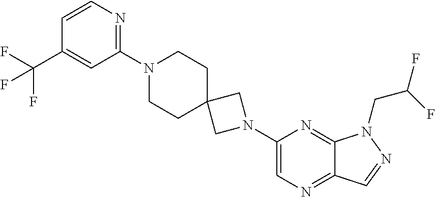 [SMILES: FC(F)Cn1ncc2ncc(N3CC4(CCN(c5cc(C(F)(F)F)ccn5)CC4)C3)nc21] | 3 |
| 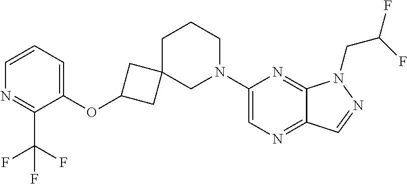 [SMILES: FC(F)Cn1ncc2ncc(N3CCCC4(CC(Oc5cccnc5C(F)(F)F)C4)C3)nc21] | 4 |
| 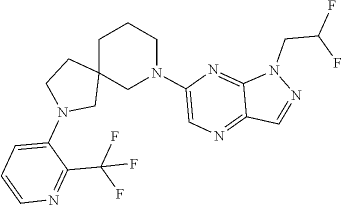 [SMILES: FC(F)Cn1ncc2ncc(N3CCCC4(CCN(c5cccnc5C(F)(F)F)C4)C3)nc21] | 5 |
| 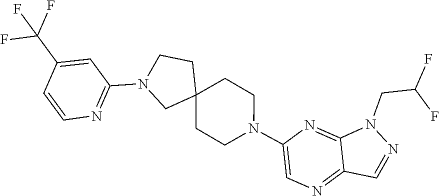 [SMILES: FC(F)Cn1ncc2ncc(N3CCC4(CCN(c5cc(C(F)(F)F)ccn5)C4)CC3)nc21] | 6 |
| 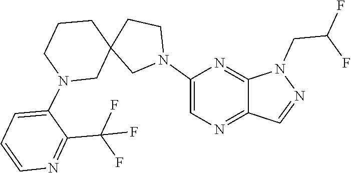 [SMILES: FC(F)Cn1ncc2ncc(N3CCC4(CCCN(c5cccnc5C(F)(F)F)C4)C3)nc21] | 7 |
| 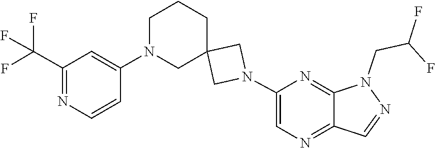 [SMILES: FC(F)Cn1ncc2ncc(N3CC4(CCCN(c5ccnc(C(F)(F)F)c5)C4)C3)nc21] | 8 |
| 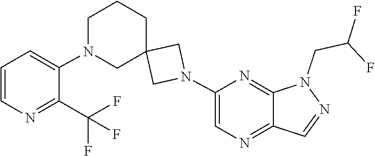 [SMILES: FC(F)Cn1ncc2ncc(N3CC4(CCCN(c5cccnc5C(F)(F)F)C4)C3)nc21] | 9 |
| 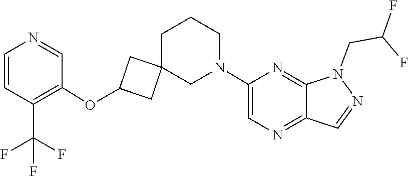 [SMILES: FC(F)Cn1ncc2ncc(N3CCCC4(CC(Oc5cnccc5C(F)(F)F)C4)C3)nc21] | 10 |
| 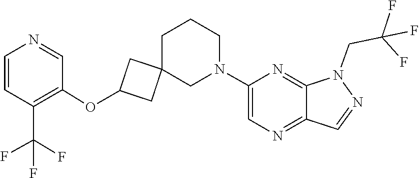 [SMILES: FC(F)(F)Cn1ncc2ncc(N3CCCC4(CC(Oc5cnccc5C(F)(F)F)C4)C3)nc21] | 11 |
| 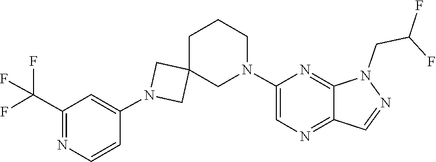 [SMILES: FC(F)Cn1ncc2ncc(N3CCCC4(CN(c5ccnc(C(F)(F)F)c5)C4)C3)nc21] | 12 |
| 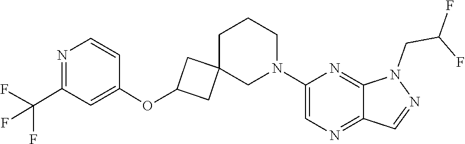 [SMILES: FC(F)Cn1ncc2ncc(N3CCCC4(CC(Oc5ccnc(C(F)(F)F)c5)C4)C3)nc21] | 13 |
| 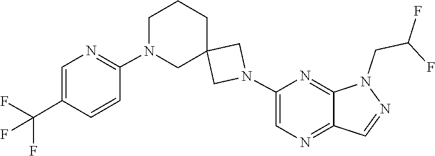 [SMILES: FC(F)Cn1ncc2ncc(N3CC4(CCCN(c5ccc(C(F)(F)F)cn5)C4)C3)nc21] | 14 |
| 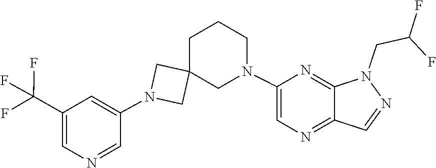 [SMILES: FC(F)Cn1ncc2ncc(N3CCCC4(CN(c5cncc(C(F)(F)F)c5)C4)C3)nc21] | 15 |
| 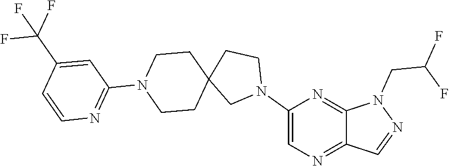 [SMILES: FC(F)Cn1ncc2ncc(N3CCC4(CCN(c5cc(C(F)(F)F)ccn5)CC4)C3)nc21] | 16 |
| 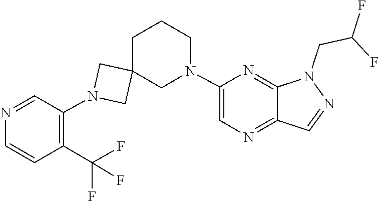 [SMILES: FC(F)Cn1ncc2ncc(N3CCCC4(C3)CN(c3cnccc3C(F)(F)F)C4)nc21] | 17 |
| 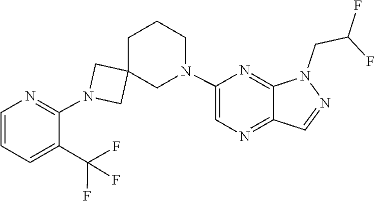 [SMILES: FC(F)Cn1ncc2ncc(N3CCCC4(C3)CN(c3ncccc3C(F)(F)F)C4)nc21] | 18 |
| 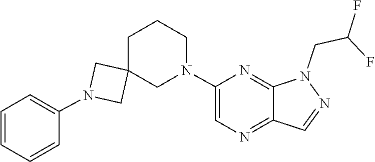 [SMILES: FC(F)Cn1ncc2ncc(N3CCCC4(CN(c5ccccc5)C4)C3)nc21] | 19 |
| 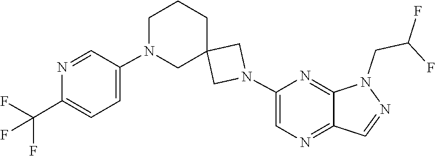 [SMILES: FC(F)Cn1ncc2ncc(N3CC4(CCCN(c5ccc(C(F)(F)F)nc5)C4)C3)nc21] | 20 |
| 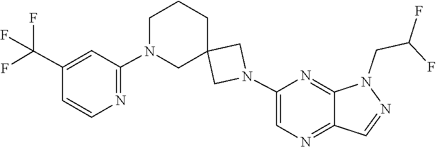 [SMILES: FC(F)Cn1ncc2ncc(N3CC4(CCCN(c5cc(C(F)(F)F)ccn5)C4)C3)nc21] | 21 |
| 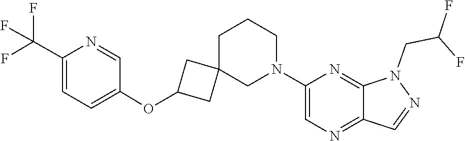 [SMILES: FC(F)Cn1ncc2ncc(N3CCCC4(CC(Oc5ccc(C(F)(F)F)nc5)C4)C3)nc21] | 22 |
| 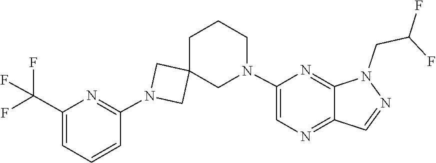 [SMILES: FC(F)Cn1ncc2ncc(N3CCCC4(CN(c5cccc(C(F)(F)F)n5)C4)C3)nc21] | 23 |
| 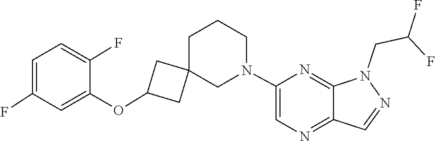 [SMILES: Fc1ccc(F)c(OC2CC3(CCCN(c4cnc5cnn(CC(F)F)c5n4)C3)C2)c1] | 24 |
| 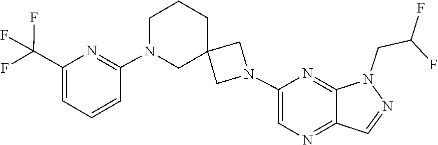 [SMILES: FC(F)Cn1ncc2ncc(N3CC4(CCCN(c5cccc(C(F)(F)F)n5)C4)C3)nc21] | 25 |
| 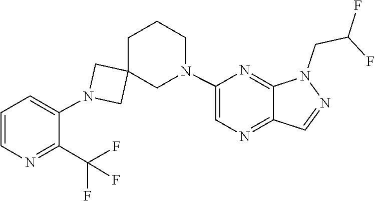 [SMILES: FC(F)Cn1ncc2ncc(N3CCCC4(C3)CN(c3cccnc3C(F)(F)F)C4)nc21] | 26 |
| 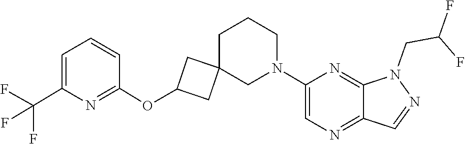 [SMILES: FC(F)Cn1ncc2ncc(N3CCCC4(CC(Oc5cccc(C(F)(F)F)n5)C4)C3)nc21] | 27 |
| 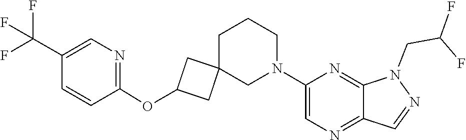 [SMILES: FC(F)Cn1ncc2ncc(N3CCCC4(CC(Oc5ccc(C(F)(F)F)cn5)C4)C3)nc21] | 28 |
| 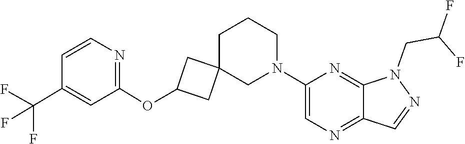 [SMILES: FC(F)Cn1ncc2ncc(N3CCCC4(CC(Oc5cc(C(F)(F)F)ccn5)C4)C3)nc21] | 29 |
| 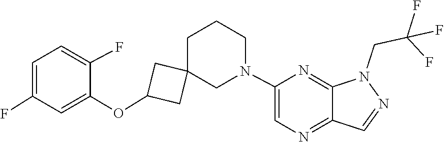 [SMILES: Fc1ccc(F)c(OC2CC3(CCCN(c4cnc5cnn(CC(F)(F)F)c5n4)C3)C2)c1] | 30 |
| 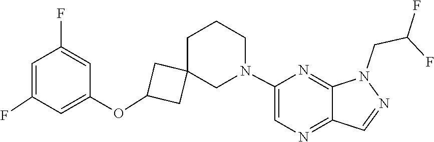 [SMILES: Fc1cc(F)cc(OC2CC3(CCCN(c4cnc5cnn(CC(F)F)c5n4)C3)C2)c1] | 31 |
| 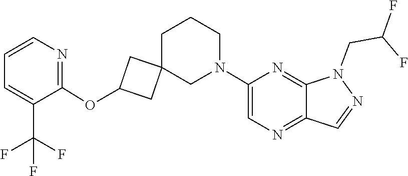 [SMILES: FC(F)Cn1ncc2ncc(N3CCCC4(CC(Oc5ncccc5C(F)(F)F)C4)C3)nc21] | 32 |
| 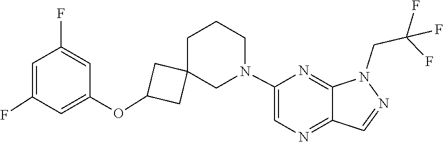 [SMILES: Fc1cc(F)cc(OC2CC3(CCCN(c4cnc5cnn(CC(F)(F)F)c5n4)C3)C2)c1] | 33 |
| 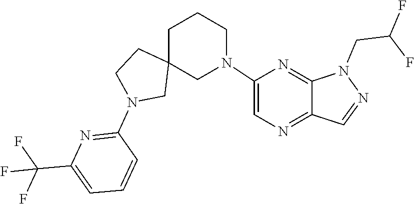 [SMILES: FC(F)Cn1ncc2ncc(N3CCCC4(CCN(c5cccc(C(F)(F)F)n5)C4)C3)nc21] | 34 |
| 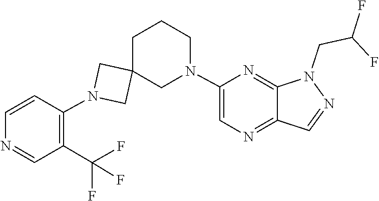 [SMILES: FC(F)Cn1ncc2ncc(N3CCCC4(C3)CN(c3ccncc3C(F)(F)F)C4)nc21] | 35 |
| 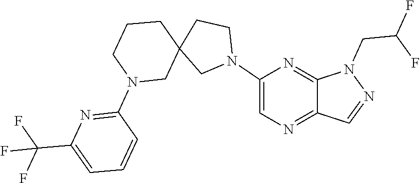 [SMILES: FC(F)Cn1ncc2ncc(N3CCC4(CCCN(c5cccc(C(F)(F)F)n5)C4)C3)nc21] | 36 |
| 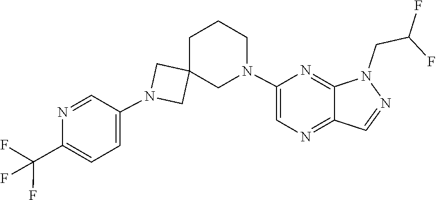 [SMILES: FC(F)Cn1ncc2ncc(N3CCCC4(CN(c5ccc(C(F)(F)F)nc5)C4)C3)nc21] | 37 |
| 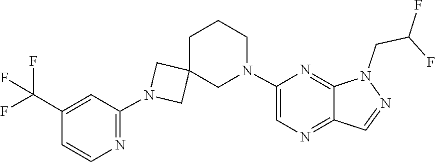 [SMILES: FC(F)Cn1ncc2ncc(N3CCCC4(CN(c5cc(C(F)(F)F)ccn5)C4)C3)nc21] | 38 |
| 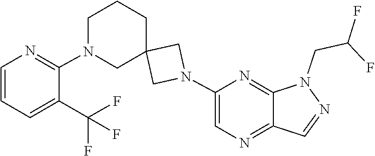 [SMILES: FC(F)Cn1ncc2ncc(N3CC4(CCCN(c5ncccc5C(F)(F)F)C4)C3)nc21] | 39 |
| 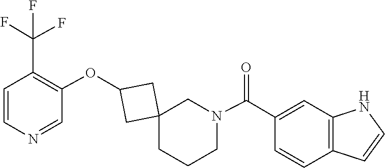 [SMILES: O=C(c1ccc2cc[nH]c2c1)N1CCCC2(CC(Oc3cnccc3C(F)(F)F)C2)C1] | 40 |
| 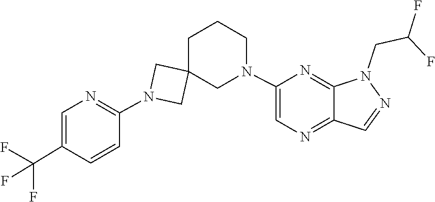 [SMILES: FC(F)Cn1ncc2ncc(N3CCCC4(CN(c5ccc(C(F)(F)F)cn5)C4)C3)nc21] | 41 |
| 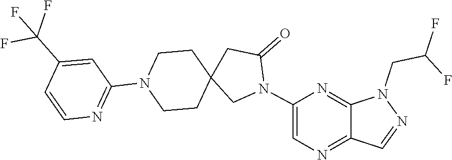 [SMILES: O=C1CC2(CCN(c3cc(C(F)(F)F)ccn3)CC2)CN1c1cnc2cnn(CC(F)F)c2n1] | 42 |
| 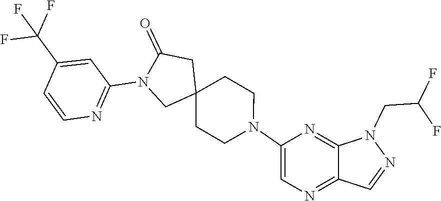 [SMILES: O=C1CC2(CCN(c3cnc4cnn(CC(F)F)c4n3)CC2)CN1c1cc(C(F)(F)F)ccn1] | 43 |
| 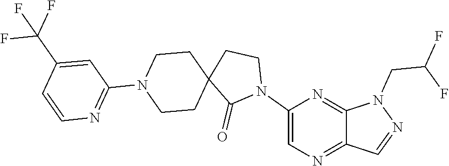 [SMILES: O=C1N(c2cnc3cnn(CC(F)F)c3n2)CCC12CCN(c1cc(C(F)(F)F)ccn1)CC2] | 44 |
| 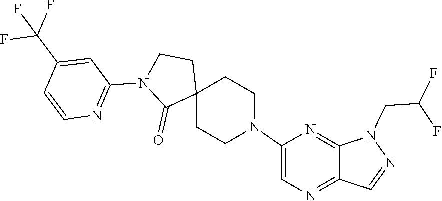 [SMILES: O=C1N(c2cc(C(F)(F)F)ccn2)CCC12CCN(c1cnc3cnn(CC(F)F)c3n1)CC2] | 45 |
| 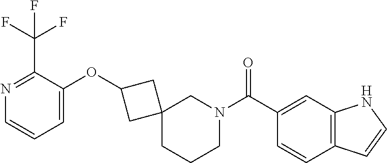 [SMILES: O=C(c1ccc2cc[nH]c2c1)N1CCCC2(CC(Oc3cccnc3C(F)(F)F)C2)C1] | 46 |
| 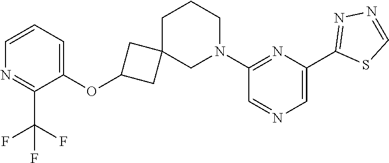 [SMILES: FC(F)(F)c1ncccc1OC1CC2(CCCN(c3cncc(-c4nncs4)n3)C2)C1] | 47 |
| 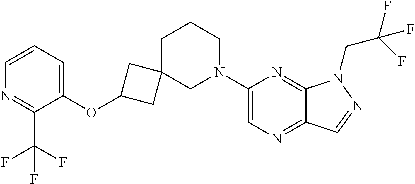 [SMILES: FC(F)(F)Cn1ncc2ncc(N3CCCC4(CC(Oc5cccnc5C(F)(F)F)C4)C3)nc21] | 48 |
| 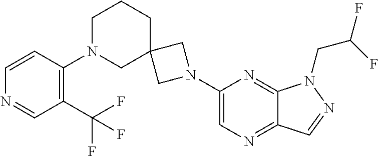 [SMILES: FC(F)Cn1ncc2ncc(N3CC4(CCCN(c5ccncc5C(F)(F)F)C4)C3)nc21] | 49 |
| 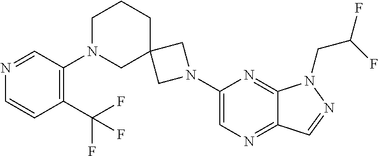 [SMILES: FC(F)Cn1ncc2ncc(N3CC4(CCCN(c5cnccc5C(F)(F)F)C4)C3)nc21] | 50 |
| 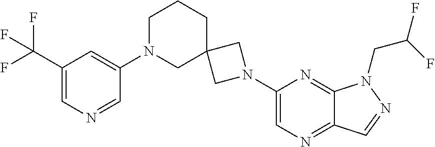 [SMILES: FC(F)Cn1ncc2ncc(N3CC4(CCCN(c5cncc(C(F)(F)F)c5)C4)C3)nc21] | 51 |
| 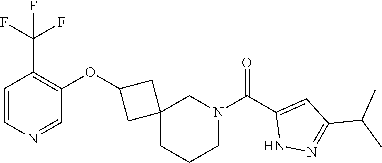 [SMILES: CC(C)c1cc(C(=O)N2CCCC3(CC(Oc4cnccc4C(F)(F)F)C3)C2)[nH]n1] | 52 |
| 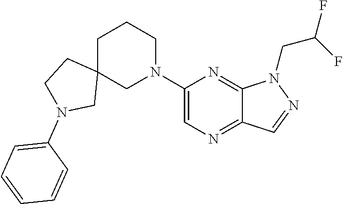 [SMILES: FC(F)Cn1ncc2ncc(N3CCCC4(CCN(c5ccccc5)C4)C3)nc21] | 53 |
| 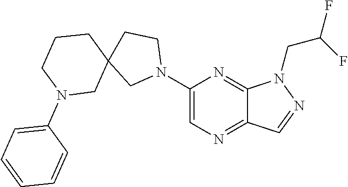 [SMILES: FC(F)Cn1ncc2ncc(N3CCC4(CCCN(c5ccccc5)C4)C3)nc21] | 54 |
| 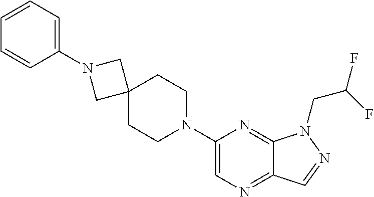 [SMILES: FC(F)Cn1ncc2ncc(N3CCC4(CC3)CN(c3ccccc3)C4)nc21] | 55 |
| 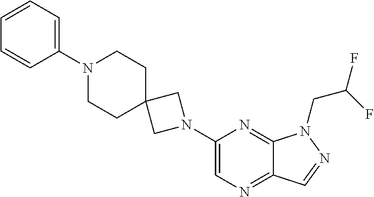 [SMILES: FC(F)Cn1ncc2ncc(N3CC4(CCN(c5ccccc5)CC4)C3)nc21] | 56 |
| 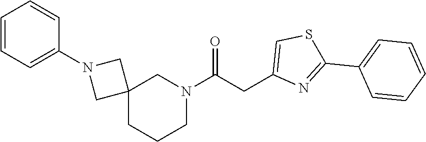 [SMILES: O=C(Cc1csc(-c2ccccc2)n1)N1CCCC2(C1)CN(c1ccccc1)C2] | 57 |
| 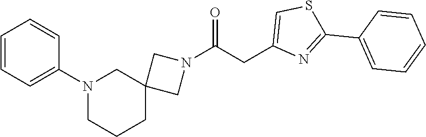 [SMILES: O=C(Cc1csc(-c2ccccc2)n1)N1CC2(CCCN(c3ccccc3)C2)C1] | 58 |
| 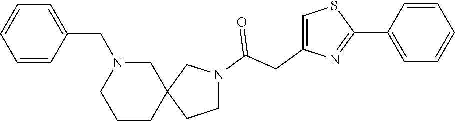 [SMILES: O=C(Cc1csc(-c2ccccc2)n1)N1CCC2(CCCN(Cc3ccccc3)C2)C1] | 59 |
| 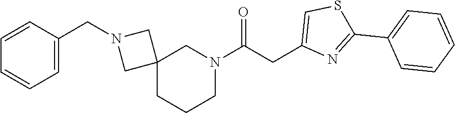 [SMILES: O=C(Cc1csc(-c2ccccc2)n1)N1CCCC2(CN(Cc3ccccc3)C2)C1] | 60 |
| 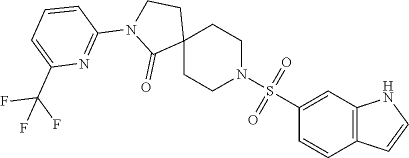 [SMILES: O=C1N(c2cccc(C(F)(F)F)n2)CCC12CCN(S(=O)(=O)c1ccc3cc[nH]c3c1)CC2] | 61 |
| 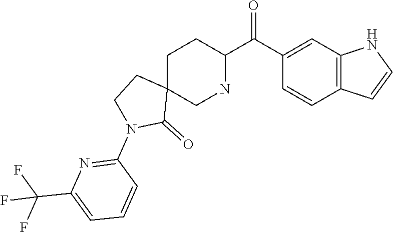 [SMILES: O=C(c1ccc2cc[nH]c2c1)C1CCC2(CCN(c3cccc(C(F)(F)F)n3)C2=O)CN1] | 62 |
| 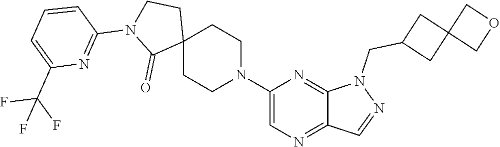 [SMILES: O=C1N(c2cccc(C(F)(F)F)n2)CCC12CCN(c1cnc3cnn(CC4CC5(COC5)C4)c3n1)CC2] | 63 |
| 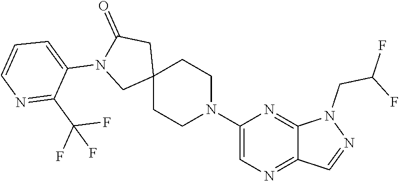 [SMILES: O=C1CC2(CCN(c3cnc4cnn(CC(F)F)c4n3)CC2)CN1c1cccnc1C(F)(F)F] | 64 |
| 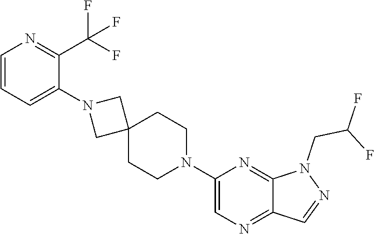 [SMILES: FC(F)Cn1ncc2ncc(N3CCC4(CC3)CN(c3cccnc3C(F)(F)F)C4)nc21] | 65 |
| 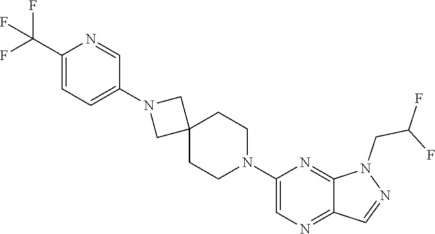 [SMILES: FC(F)Cn1ncc2ncc(N3CCC4(CC3)CN(c3ccc(C(F)(F)F)nc3)C4)nc21] | 66 |
| 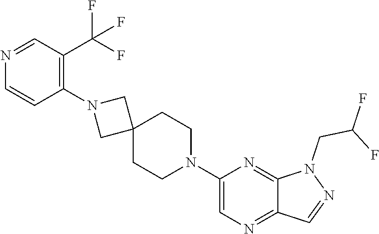 [SMILES: FC(F)Cn1ncc2ncc(N3CCC4(CC3)CN(c3ccncc3C(F)(F)F)C4)nc21] | 67 |
| 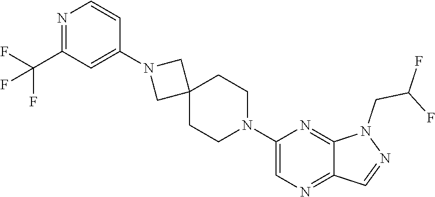 [SMILES: FC(F)Cn1ncc2ncc(N3CCC4(CC3)CN(c3ccnc(C(F)(F)F)c3)C4)nc21] | 68 |
| 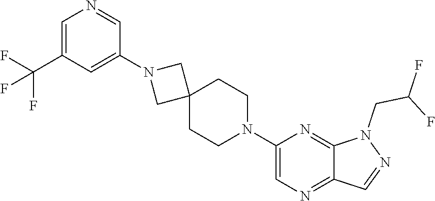 [SMILES: FC(F)Cn1ncc2ncc(N3CCC4(CC3)CN(c3cncc(C(F)(F)F)c3)C4)nc21] | 69 |
| 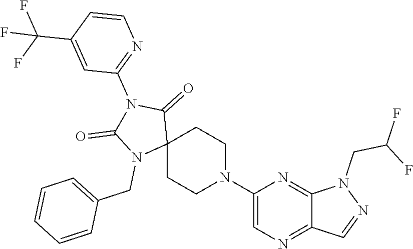 [SMILES: O=C1N(c2cc(C(F)(F)F)ccn2)C(=O)C2(CCN(c3cnc4cnn(CC(F)F)c4n3)CC2)N1Cc1ccccc1] | 70 |
| 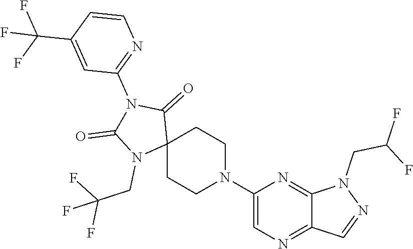 [SMILES: O=C1N(c2cc(C(F)(F)F)ccn2)C(=O)C2(CCN(c3cnc4cnn(CC(F)F)c4n3)CC2)N1CC(F)(F)F] | 71 |
| 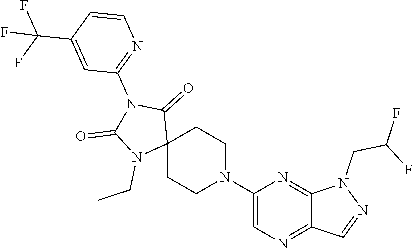 [SMILES: CCN1C(=O)N(c2cc(C(F)(F)F)ccn2)C(=O)C12CCN(c1cnc3cnn(CC(F)F)c3n1)CC2] | 72 |
| 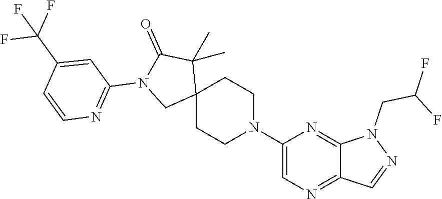 [SMILES: CC1(C)C(=O)N(c2cc(C(F)(F)F)ccn2)CC12CCN(c1cnc3cnn(CC(F)F)c3n1)CC2] | 73 |
| 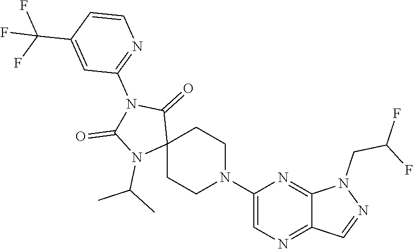 [SMILES: CC(C)N1C(=O)N(c2cc(C(F)(F)F)ccn2)C(=O)C12CCN(c1cnc3cnn(CC(F)F)c3n1)CC2] | 74 |
| 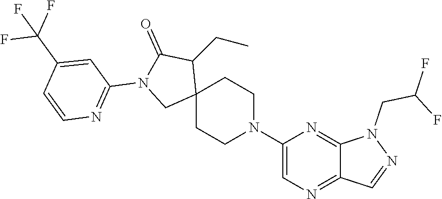 [SMILES: CCC1C(=O)N(c2cc(C(F)(F)F)ccn2)CC12CCN(c1cnc3cnn(CC(F)F)c3n1)CC2] | 75 |
| 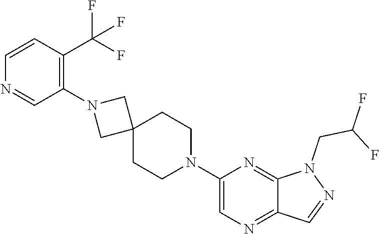 [SMILES: FC(F)Cn1ncc2ncc(N3CCC4(CC3)CN(c3cnccc3C(F)(F)F)C4)nc21] | 76 |
| 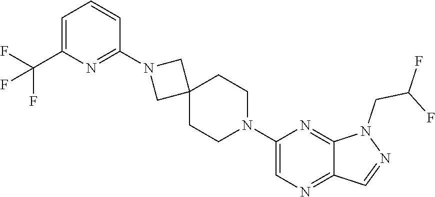 [SMILES: FC(F)Cn1ncc2ncc(N3CCC4(CC3)CN(c3cccc(C(F)(F)F)n3)C4)nc21] | 77 |
| 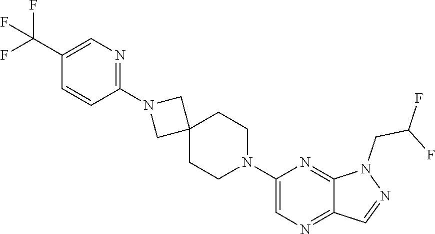 [SMILES: FC(F)Cn1ncc2ncc(N3CCC4(CC3)CN(c3ccc(C(F)(F)F)cn3)C4)nc21] | 78 |
| 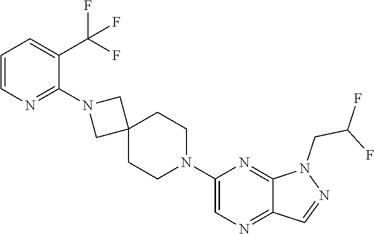 [SMILES: FC(F)Cn1ncc2ncc(N3CCC4(CC3)CN(c3ncccc3C(F)(F)F)C4)nc21] | 79 |
| 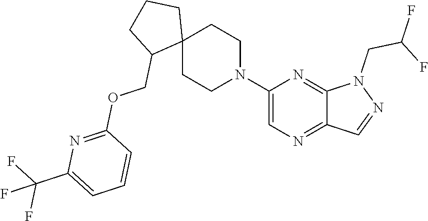 [SMILES: FC(F)Cn1ncc2ncc(N3CCC4(CCCC4COc4cccc(C(F)(F)F)n4)CC3)nc21] | 80 |
| 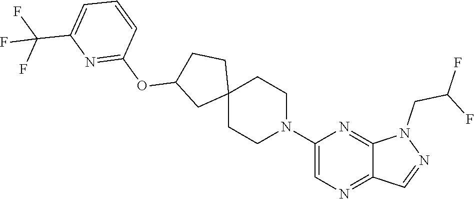 [SMILES: FC(F)Cn1ncc2ncc(N3CCC4(CCC(Oc5cccc(C(F)(F)F)n5)C4)CC3)nc21] | 81 |
| 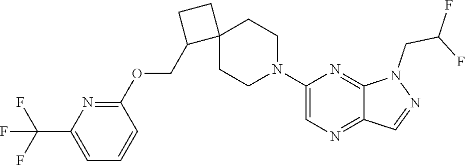 [SMILES: FC(F)Cn1ncc2ncc(N3CCC4(CCC4COc4cccc(C(F)(F)F)n4)CC3)nc21] | 82 |
| 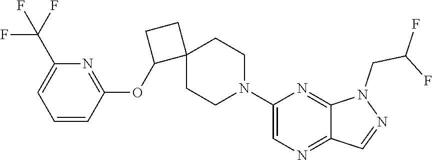 [SMILES: FC(F)Cn1ncc2ncc(N3CCC4(CCC4Oc4cccc(C(F)(F)F)n4)CC3)nc21] | 83 |
| 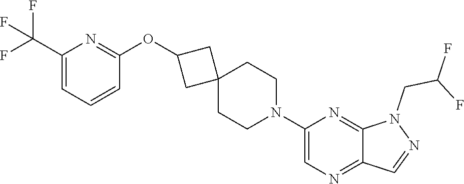 [SMILES: FC(F)Cn1ncc2ncc(N3CCC4(CC3)CC(Oc3cccc(C(F)(F)F)n3)C4)nc21] | 84 |
| 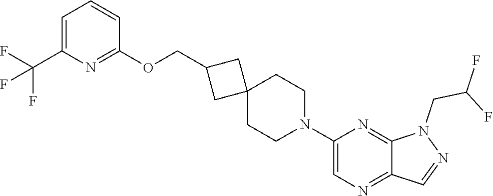 [SMILES: FC(F)Cn1ncc2ncc(N3CCC4(CC3)CC(COc3cccc(C(F)(F)F)n3)C4)nc21] | 85 |
| 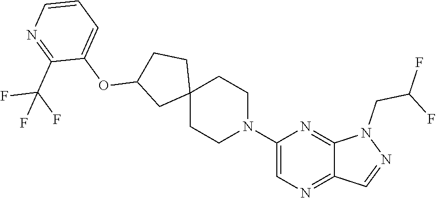 [SMILES: FC(F)Cn1ncc2ncc(N3CCC4(CCC(Oc5cccnc5C(F)(F)F)C4)CC3)nc21] | 86 |
| 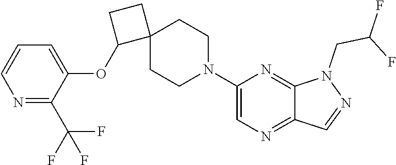 [SMILES: FC(F)Cn1ncc2ncc(N3CCC4(CCC4Oc4cccnc4C(F)(F)F)CC3)nc21] | 87 |
| 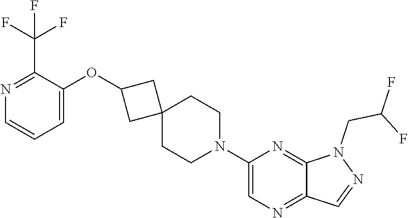 [SMILES: FC(F)Cn1ncc2ncc(N3CCC4(CC3)CC(Oc3cccnc3C(F)(F)F)C4)nc21] | 88 |
| 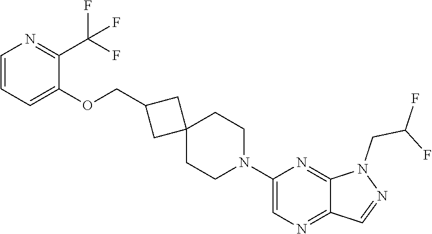 [SMILES: FC(F)Cn1ncc2ncc(N3CCC4(CC3)CC(COc3cccnc3C(F)(F)F)C4)nc21] | 89 |
| 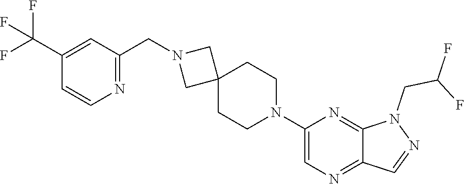 [SMILES: FC(F)Cn1ncc2ncc(N3CCC4(CC3)CN(Cc3cc(C(F)(F)F)ccn3)C4)nc21] | 90 |
| 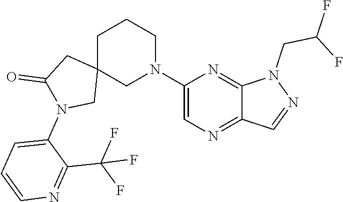 [SMILES: O=C1CC2(CCCN(c3cnc4cnn(CC(F)F)c4n3)C2)CN1c1cccnc1C(F)(F)F] | 91 |
| 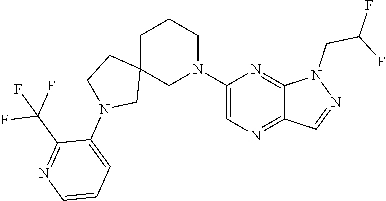 [SMILES: FC(F)Cn1ncc2ncc(N3CCCC4(CCN(c5cccnc5C(F)(F)F)C4)C3)nc21] | 92 |
| 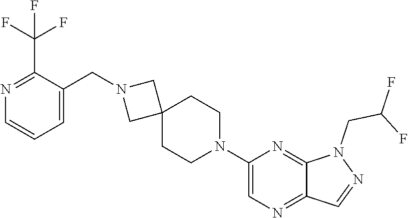 [SMILES: FC(F)Cn1ncc2ncc(N3CCC4(CC3)CN(Cc3cccnc3C(F)(F)F)C4)nc21] | 93 |
| 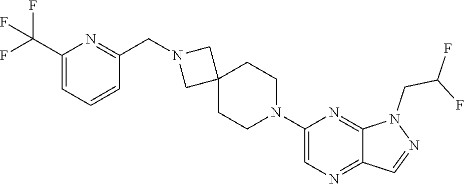 [SMILES: FC(F)Cn1ncc2ncc(N3CCC4(CC3)CN(Cc3cccc(C(F)(F)F)n3)C4)nc21] | 94 |
| 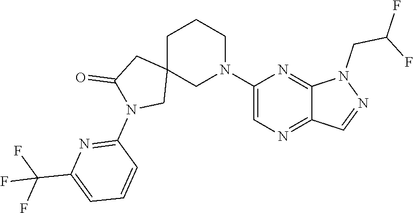 [SMILES: O=C1CC2(CCCN(c3cnc4cnn(CC(F)F)c4n3)C2)CN1c1cccc(C(F)(F)F)n1] | 95 |
| 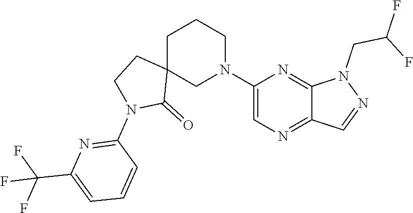 [SMILES: O=C1N(c2cccc(C(F)(F)F)n2)CCC12CCCN(c1cnc3cnn(CC(F)F)c3n1)C2] | 96 |
| 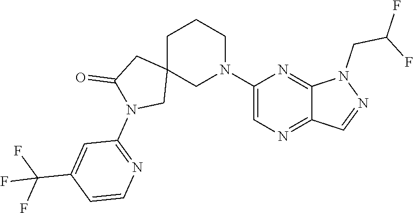 [SMILES: O=C1CC2(CCCN(c3cnc4cnn(CC(F)F)c4n3)C2)CN1c1cc(C(F)(F)F)ccn1] | 97 |
| 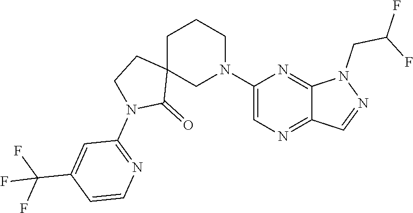 [SMILES: O=C1N(c2cc(C(F)(F)F)ccn2)CCC12CCCN(c1cnc3cnn(CC(F)F)c3n1)C2] | 98 |
| 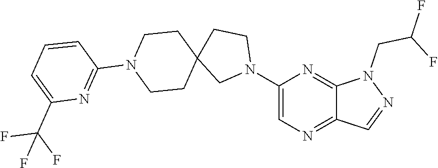 [SMILES: FC(F)Cn1ncc2ncc(N3CCC4(CCN(c5cccc(C(F)(F)F)n5)CC4)C3)nc21] | 99 |
| 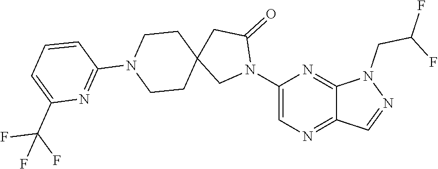 [SMILES: O=C1CC2(CCN(c3cccc(C(F)(F)F)n3)CC2)CN1c1cnc2cnn(CC(F)F)c2n1] | 100 |
| 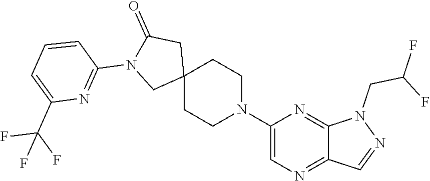 [SMILES: O=C1CC2(CCN(c3cnc4cnn(CC(F)F)c4n3)CC2)CN1c1cccc(C(F)(F)F)n1] | 101 |
| 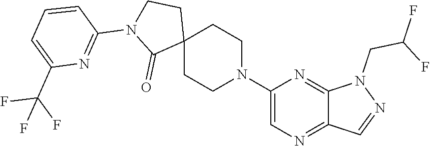 [SMILES: O=C1N(c2cccc(C(F)(F)F)n2)CCC12CCN(c1cnc3cnn(CC(F)F)c3n1)CC2] | 102 |
| 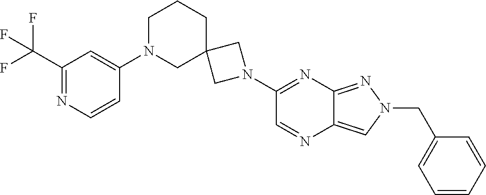 [SMILES: FC(F)(F)c1cc(N2CCCC3(C2)CN(c2cnc4cn(Cc5ccccc5)nc4n2)C3)ccn1] | 103 |
| 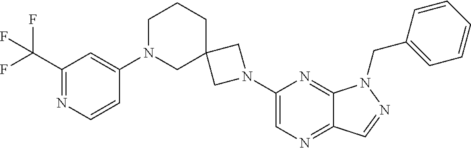 [SMILES: FC(F)(F)c1cc(N2CCCC3(C2)CN(c2cnc4cnn(Cc5ccccc5)c4n2)C3)ccn1] | 104 |
| 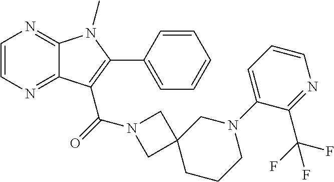 [SMILES: Cn1c(-c2ccccc2)c(C(=O)N2CC3(CCCN(c4cccnc4C(F)(F)F)C3)C2)c2nccnc21] | 105 |
| 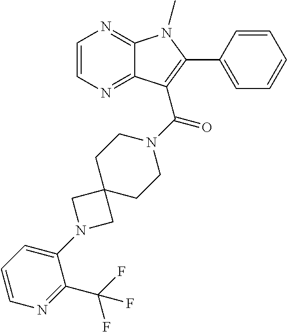 [SMILES: Cn1c(-c2ccccc2)c(C(=O)N2CCC3(CC2)CN(c2cccnc2C(F)(F)F)C3)c2nccnc21] | 106 |
| 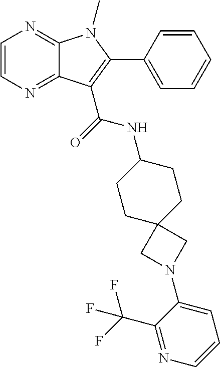 [SMILES: Cn1c(-c2ccccc2)c(C(=O)NC2CCC3(CC2)CN(c2cccnc2C(F)(F)F)C3)c2nccnc21] | 107 |
| 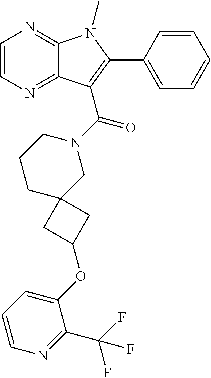 [SMILES: Cn1c(-c2ccccc2)c(C(=O)N2CCCC3(CC(Oc4cccnc4C(F)(F)F)C3)C2)c2nccnc21] | 108 |
| 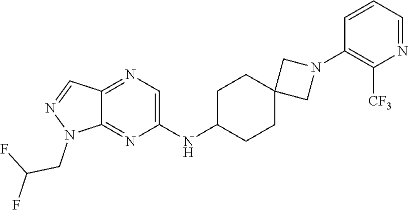 [SMILES: FC(F)Cn1ncc2ncc(NC3CCC4(CC3)CN(c3cccnc3C(F)(F)F)C4)nc21] | 109 |
| 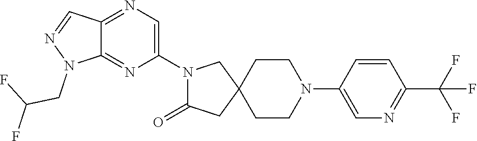 [SMILES: O=C1CC2(CCN(c3ccc(C(F)(F)F)nc3)CC2)CN1c1cnc2cnn(CC(F)F)c2n1] | 110 |
| 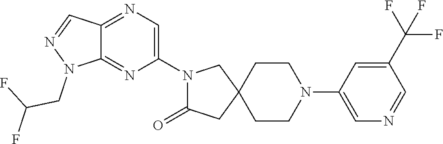 [SMILES: O=C1CC2(CCN(c3cncc(C(F)(F)F)c3)CC2)CN1c1cnc2cnn(CC(F)F)c2n1] | 111 |
| 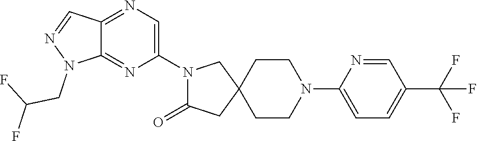 [SMILES: O=C1CC2(CCN(c3ccc(C(F)(F)F)cn3)CC2)CN1c1cnc2cnn(CC(F)F)c2n1] | 112 |
| 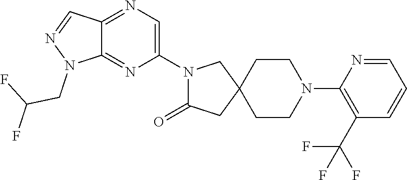 [SMILES: O=C1CC2(CCN(c3ncccc3C(F)(F)F)CC2)CN1c1cnc2cnn(CC(F)F)c2n1] | 113 |
| 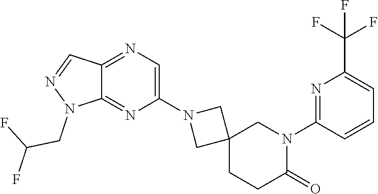 [SMILES: O=C1CCC2(CN(c3cnc4cnn(CC(F)F)c4n3)C2)CN1c1cccc(C(F)(F)F)n1] | 114 |
| 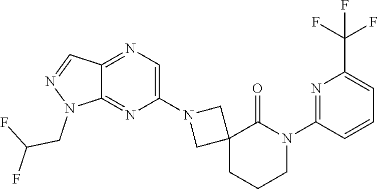 [SMILES: O=C1N(c2cccc(C(F)(F)F)n2)CCCC12CN(c1cnc3cnn(CC(F)F)c3n1)C2] | 115 |
| 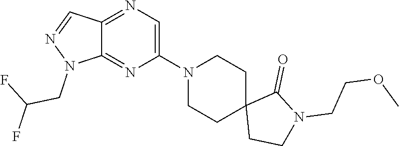 [SMILES: COCCN1CCC2(CCN(c3cnc4cnn(CC(F)F)c4n3)CC2)C1=O] | 116 |
| 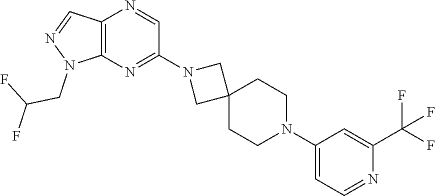 [SMILES: FC(F)Cn1ncc2ncc(N3CC4(CCN(c5ccnc(C(F)(F)F)c5)CC4)C3)nc21] | 117 |
| 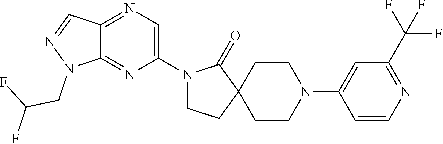 [SMILES: O=C1N(c2cnc3cnn(CC(F)F)c3n2)CCC12CCN(c1ccnc(C(F)(F)F)c1)CC2] | 118 |
| 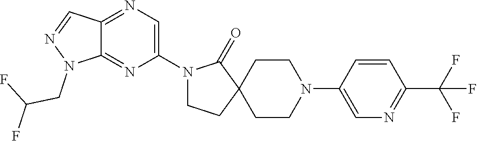 [SMILES: O=C1N(c2cnc3cnn(CC(F)F)c3n2)CCC12CCN(c1ccc(C(F)(F)F)nc1)CC2] | 119 |
| 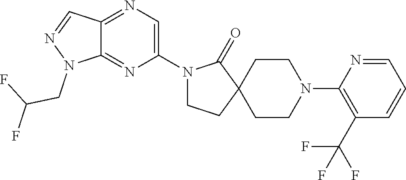 [SMILES: O=C1N(c2cnc3cnn(CC(F)F)c3n2)CCC12CCN(c1ncccc1C(F)(F)F)CC2] | 120 |
| 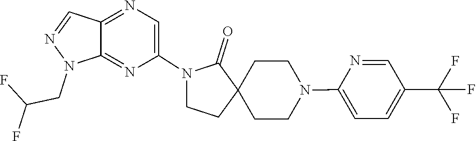 [SMILES: O=C1N(c2cnc3cnn(CC(F)F)c3n2)CCC12CCN(c1ccc(C(F)(F)F)cn1)CC2] | 121 |
| 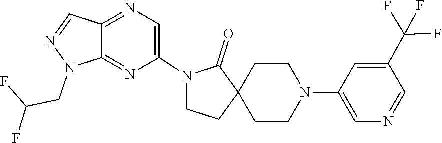 [SMILES: O=C1N(c2cnc3cnn(CC(F)F)c3n2)CCC12CCN(c1cncc(C(F)(F)F)c1)CC2] | 122 |
| 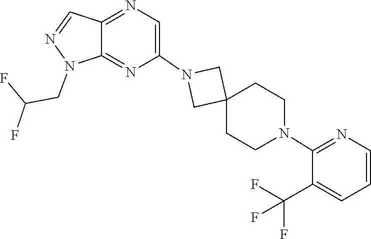 [SMILES: FC(F)Cn1ncc2ncc(N3CC4(CCN(c5ncccc5C(F)(F)F)CC4)C3)nc21] | 123 |
| 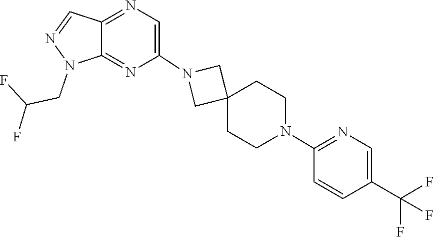 [SMILES: FC(F)Cn1ncc2ncc(N3CC4(CCN(c5ccc(C(F)(F)F)cn5)CC4)C3)nc21] | 124 |
| 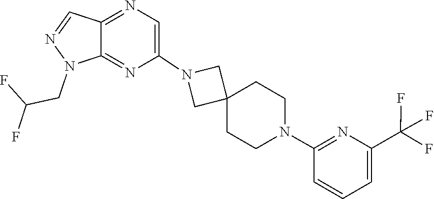 [SMILES: FC(F)Cn1ncc2ncc(N3CC4(CCN(c5cccc(C(F)(F)F)n5)CC4)C3)nc21] | 125 |
| 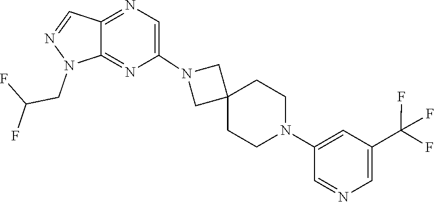 [SMILES: FC(F)Cn1ncc2ncc(N3CC4(CCN(c5cncc(C(F)(F)F)c5)CC4)C3)nc21] | 126 |
| 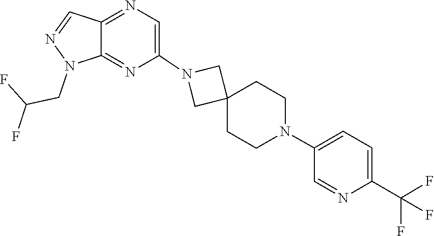 [SMILES: FC(F)Cn1ncc2ncc(N3CC4(CCN(c5ccc(C(F)(F)F)nc5)CC4)C3)nc21] | 127 |
| 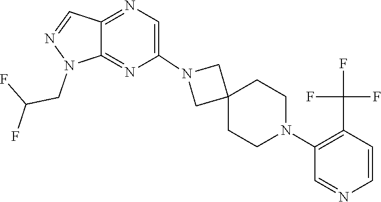 [SMILES: FC(F)Cn1ncc2ncc(N3CC4(CCN(c5cnccc5C(F)(F)F)CC4)C3)nc21] | 128 |
| 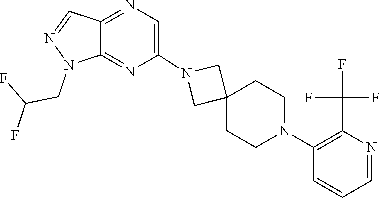 [SMILES: FC(F)Cn1ncc2ncc(N3CC4(CCN(c5cccnc5C(F)(F)F)CC4)C3)nc21] | 129 |
| 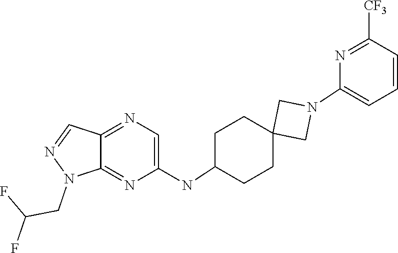 | 130 |
| 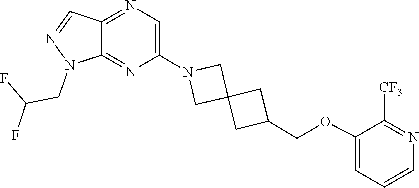 [SMILES: FC(F)Cn1ncc2ncc(N3CC4(CC(COc5cccnc5C(F)(F)F)C4)C3)nc21] | 131 |
| 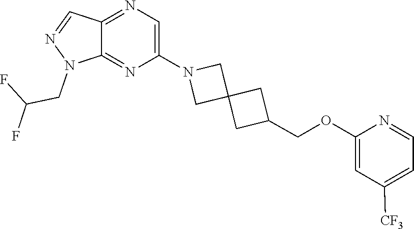 [SMILES: FC(F)Cn1ncc2ncc(N3CC4(CC(COc5cc(C(F)(F)F)ccn5)C4)C3)nc21] | 132 |
| 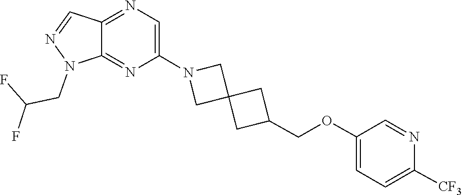 [SMILES: FC(F)Cn1ncc2ncc(N3CC4(CC(COc5ccc(C(F)(F)F)nc5)C4)C3)nc21] | 133 |
| 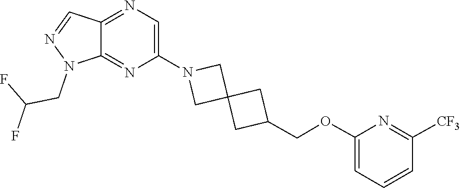 [SMILES: FC(F)Cn1ncc2ncc(N3CC4(CC(COc5cccc(C(F)(F)F)n5)C4)C3)nc21] | 134 |
| 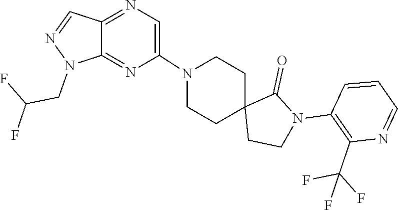 [SMILES: O=C1N(c2cccnc2C(F)(F)F)CCC12CCN(c1cnc3cnn(CC(F)F)c3n1)CC2] | 135 |
| 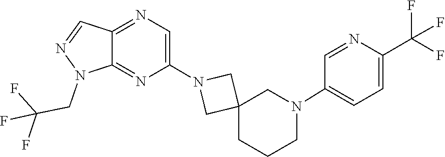 [SMILES: FC(F)(F)Cn1ncc2ncc(N3CC4(CCCN(c5ccc(C(F)(F)F)nc5)C4)C3)nc21] | 136 |
| 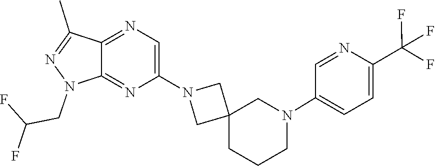 [SMILES: Cc1nn(CC(F)F)c2nc(N3CC4(CCCN(c5ccc(C(F)(F)F)nc5)C4)C3)cnc12] | 137 |
| 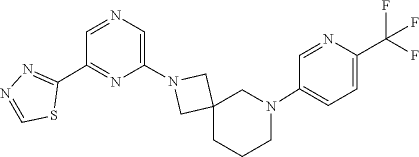 [SMILES: FC(F)(F)c1ccc(N2CCCC3(C2)CN(c2cncc(-c4nncs4)n2)C3)cn1] | 138 |
| 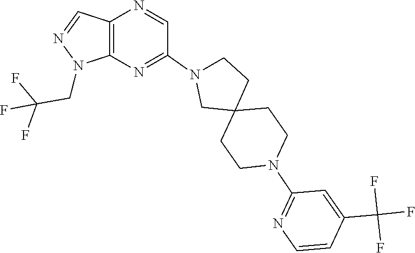 [SMILES: FC(F)(F)Cn1ncc2ncc(N3CCC4(CCN(c5cc(C(F)(F)F)ccn5)CC4)C3)nc21] | 139 |
| 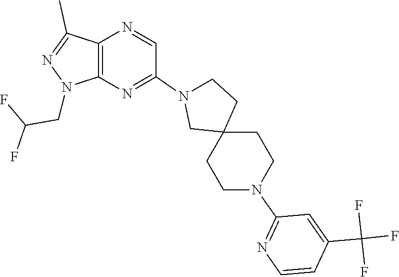 [SMILES: Cc1nn(CC(F)F)c2nc(N3CCC4(CCN(c5cc(C(F)(F)F)ccn5)CC4)C3)cnc12] | 140 |
| 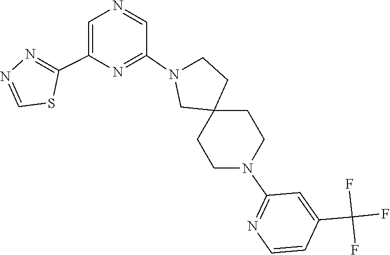 [SMILES: FC(F)(F)c1ccnc(N2CCC3(CC2)CCN(c2cncc(-c4nncs4)n2)C3)c1] | 141 |
| 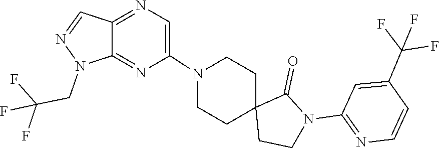 [SMILES: O=C1N(c2cc(C(F)(F)F)ccn2)CCC12CCN(c1cnc3cnn(CC(F)(F)F)c3n1)CC2] | 142 |
| 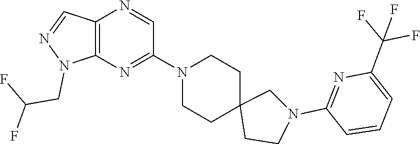 [SMILES: FC(F)Cn1ncc2ncc(N3CCC4(CCN(c5cccc(C(F)(F)F)n5)C4)CC3)nc21] | 143 |
| 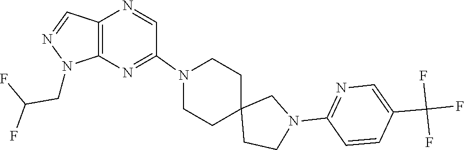 [SMILES: FC(F)Cn1ncc2ncc(N3CCC4(CCN(c5ccc(C(F)(F)F)cn5)C4)CC3)nc21] | 144 |
| 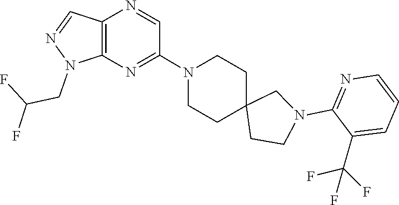 [SMILES: FC(F)Cn1ncc2ncc(N3CCC4(CC3)CCN(c3ncccc3C(F)(F)F)C4)nc21] | 145 |
| 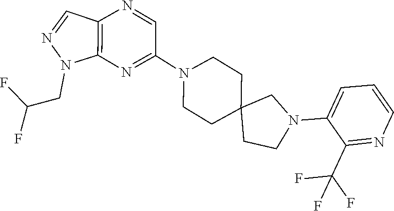 [SMILES: FC(F)Cn1ncc2ncc(N3CCC4(CC3)CCN(c3cccnc3C(F)(F)F)C4)nc21] | 146 |
| 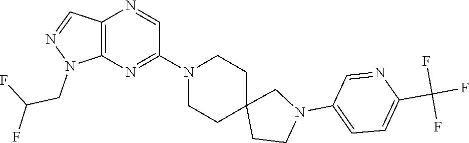 [SMILES: FC(F)Cn1ncc2ncc(N3CCC4(CCN(c5ccc(C(F)(F)F)nc5)C4)CC3)nc21] | 147 |
| 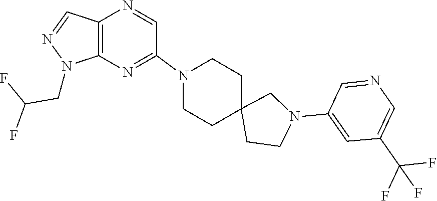 [SMILES: FC(F)Cn1ncc2ncc(N3CCC4(CCN(c5cncc(C(F)(F)F)c5)C4)CC3)nc21] | 148 |
| 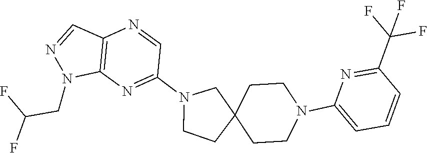 [SMILES: FC(F)Cn1ncc2ncc(N3CCC4(CCN(c5cccc(C(F)(F)F)n5)CC4)C3)nc21] | 149 |
| 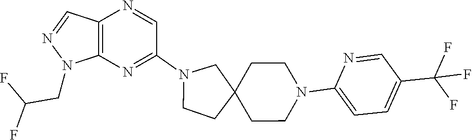 [SMILES: FC(F)Cn1ncc2ncc(N3CCC4(CCN(c5ccc(C(F)(F)F)cn5)CC4)C3)nc21] | 150 |
| 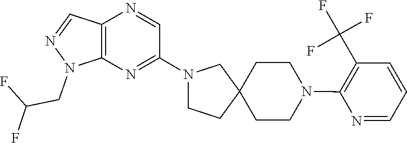 [SMILES: FC(F)Cn1ncc2ncc(N3CCC4(CCN(c5ncccc5C(F)(F)F)CC4)C3)nc21] | 151 |
| 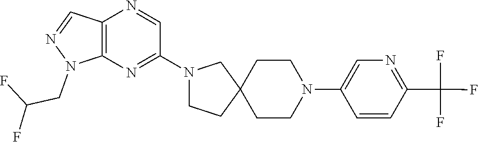 [SMILES: FC(F)Cn1ncc2ncc(N3CCC4(CCN(c5ccc(C(F)(F)F)nc5)CC4)C3)nc21] | 152 |
| 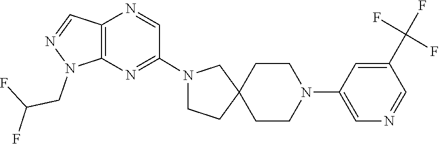 [SMILES: FC(F)Cn1ncc2ncc(N3CCC4(CCN(c5cncc(C(F)(F)F)c5)CC4)C3)nc21] | 153 |
| 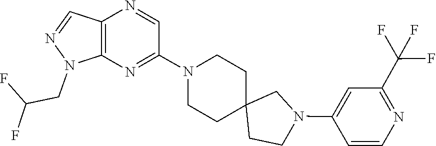 [SMILES: FC(F)Cn1ncc2ncc(N3CCC4(CCN(c5ccnc(C(F)(F)F)c5)C4)CC3)nc21] | 154 |
| 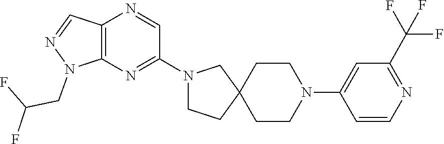 [SMILES: FC(F)Cn1ncc2ncc(N3CCC4(CCN(c5ccnc(C(F)(F)F)c5)CC4)C3)nc21] | 155 |
| 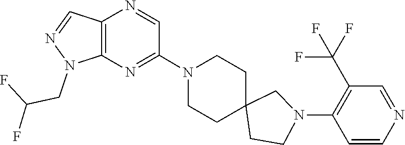 [SMILES: FC(F)Cn1ncc2ncc(N3CCC4(CC3)CCN(c3ccncc3C(F)(F)F)C4)nc21] | 156 |
| 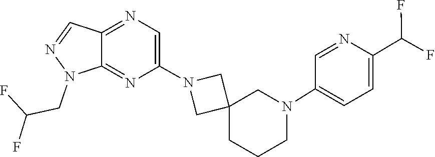 [SMILES: FC(F)Cn1ncc2ncc(N3CC4(CCCN(c5ccc(C(F)F)nc5)C4)C3)nc21] | 157 |
| 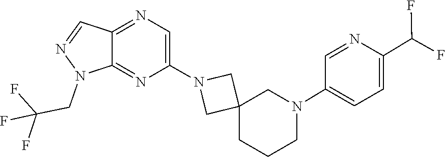 [SMILES: FC(F)c1ccc(N2CCCC3(C2)CN(c2cnc4cnn(CC(F)(F)F)c4n2)C3)cn1] | 158 |
| 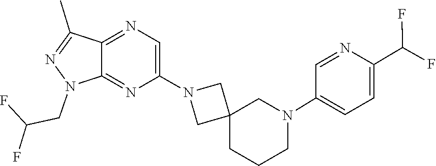 [SMILES: Cc1nn(CC(F)F)c2nc(N3CC4(CCCN(c5ccc(C(F)F)nc5)C4)C3)cnc12] | 159 |
| 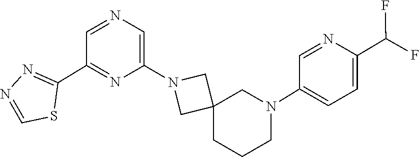 [SMILES: FC(F)c1ccc(N2CCCC3(C2)CN(c2cncc(-c4nncs4)n2)C3)cn1] | 160 |
| 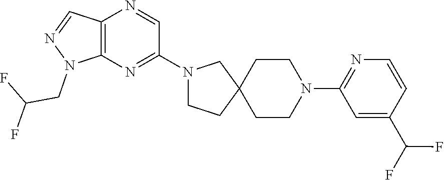 [SMILES: FC(F)Cn1ncc2ncc(N3CCC4(CCN(c5cc(C(F)F)ccn5)CC4)C3)nc21] | 161 |
| 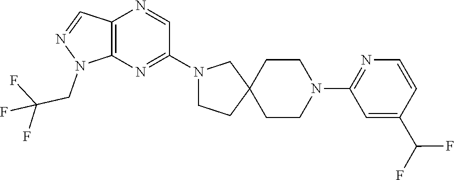 [SMILES: FC(F)c1ccnc(N2CCC3(CC2)CCN(c2cnc4cnn(CC(F)(F)F)c4n2)C3)c1] | 162 |
| 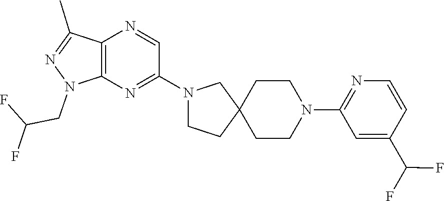 [SMILES: Cc1nn(CC(F)F)c2nc(N3CCC4(CCN(c5cc(C(F)F)ccn5)CC4)C3)cnc12] | 163 |
| 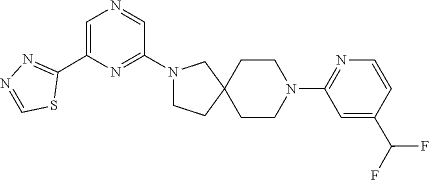 [SMILES: FC(F)c1ccnc(N2CCC3(CC2)CCN(c2cncc(-c4nncs4)n2)C3)c1] | 164 |
| 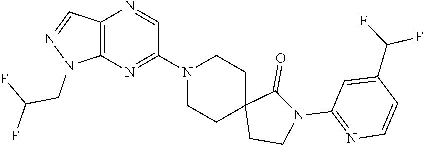 [SMILES: O=C1N(c2cc(C(F)F)ccn2)CCC12CCN(c1cnc3cnn(CC(F)F)c3n1)CC2] | 165 |
| 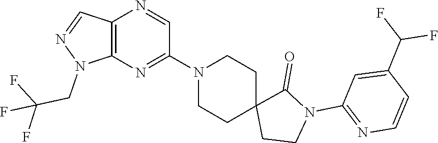 [SMILES: O=C1N(c2cc(C(F)F)ccn2)CCC12CCN(c1cnc3cnn(CC(F)(F)F)c3n1)CC2] | 166 |
| 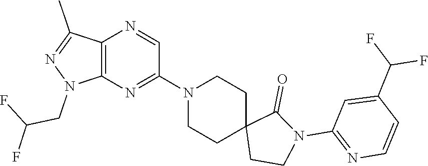 [SMILES: Cc1nn(CC(F)F)c2nc(N3CCC4(CC3)CCN(c3cc(C(F)F)ccn3)C4=O)cnc12] | 167 |
| 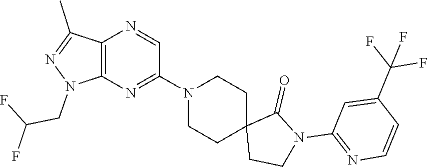 [SMILES: Cc1nn(CC(F)F)c2nc(N3CCC4(CC3)CCN(c3cc(C(F)(F)F)ccn3)C4=O)cnc12] | 168 |
| 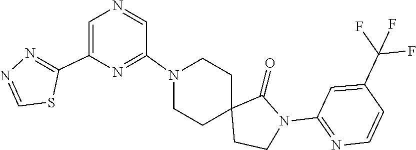 [SMILES: O=C1N(c2cc(C(F)(F)F)ccn2)CCC12CCN(c1cncc(-c3nncs3)n1)CC2] | 169 |
| 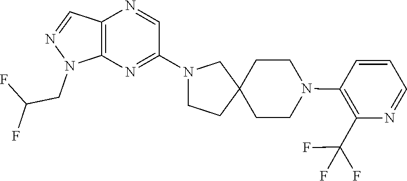 [SMILES: FC(F)Cn1ncc2ncc(N3CCC4(CCN(c5cccnc5C(F)(F)F)CC4)C3)nc21] | 170 |
| 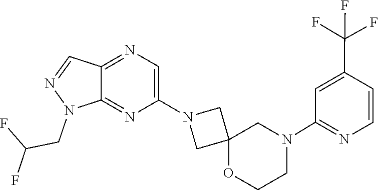 [SMILES: FC(F)Cn1ncc2ncc(N3CC4(CN(c5cc(C(F)(F)F)ccn5)CCO4)C3)nc21] | 171 |
| 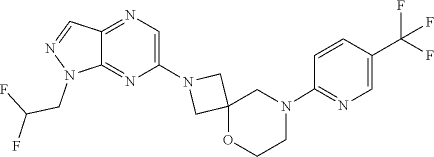 [SMILES: FC(F)Cn1ncc2ncc(N3CC4(CN(c5ccc(C(F)(F)F)cn5)CCO4)C3)nc21] | 172 |
| 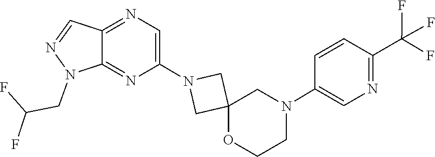 [SMILES: FC(F)Cn1ncc2ncc(N3CC4(CN(c5ccc(C(F)(F)F)nc5)CCO4)C3)nc21] | 173 |
| 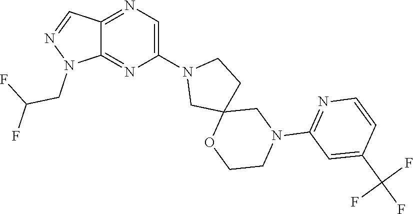 [SMILES: FC(F)Cn1ncc2ncc(N3CCC4(CN(c5cc(C(F)(F)F)ccn5)CCO4)C3)nc21] | 174 |
| 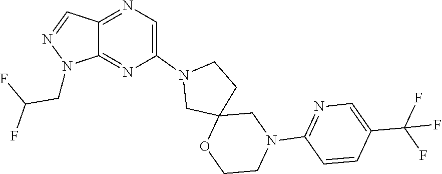 [SMILES: FC(F)Cn1ncc2ncc(N3CCC4(CN(c5ccc(C(F)(F)F)cn5)CCO4)C3)nc21] | 175 |
| 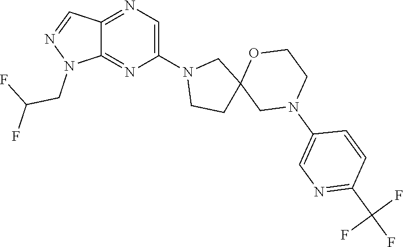 [SMILES: FC(F)Cn1ncc2ncc(N3CCC4(CN(c5ccc(C(F)(F)F)nc5)CCO4)C3)nc21] | 176 |
| 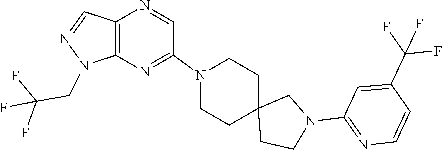 [SMILES: FC(F)(F)Cn1ncc2ncc(N3CCC4(CCN(c5cc(C(F)(F)F)ccn5)C4)CC3)nc21] | 177 |
| 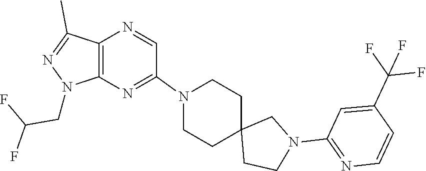 [SMILES: Cc1nn(CC(F)F)c2nc(N3CCC4(CCN(c5cc(C(F)(F)F)ccn5)C4)CC3)cnc12] | 178 |
| 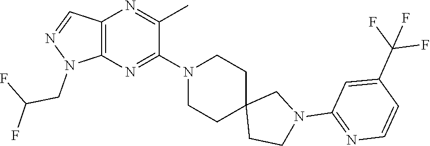 [SMILES: Cc1nc2cnn(CC(F)F)c2nc1N1CCC2(CCN(c3cc(C(F)(F)F)ccn3)C2)CC1] | 179 |
| 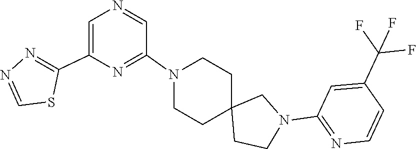 [SMILES: FC(F)(F)c1ccnc(N2CCC3(CCN(c4cncc(-c5nncs5)n4)CC3)C2)c1] | 180 |
| 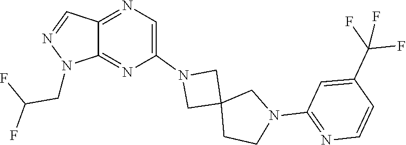 [SMILES: FC(F)Cn1ncc2ncc(N3CC4(CCN(c5cc(C(F)(F)F)ccn5)C4)C3)nc21] | 181 |
| 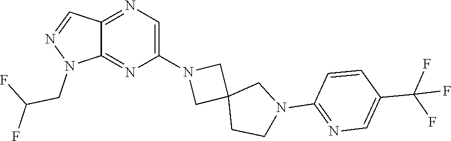 [SMILES: FC(F)Cn1ncc2ncc(N3CC4(CCN(c5ccc(C(F)(F)F)cn5)C4)C3)nc21] | 182 |
| 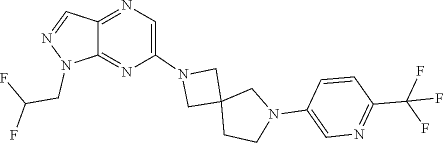 [SMILES: FC(F)Cn1ncc2ncc(N3CC4(CCN(c5ccc(C(F)(F)F)nc5)C4)C3)nc21] | 183 |
| 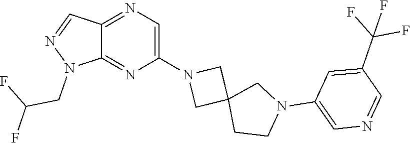 [SMILES: FC(F)Cn1ncc2ncc(N3CC4(CCN(c5cncc(C(F)(F)F)c5)C4)C3)nc21] | 184 |
| 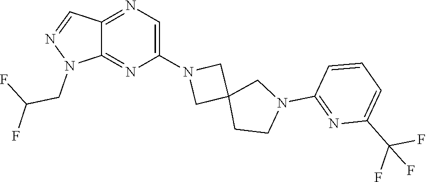 [SMILES: FC(F)Cn1ncc2ncc(N3CC4(CCN(c5cccc(C(F)(F)F)n5)C4)C3)nc21] | 185 |
| 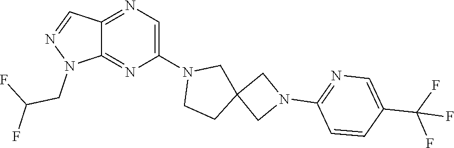 [SMILES: FC(F)Cn1ncc2ncc(N3CCC4(CN(c5ccc(C(F)(F)F)cn5)C4)C3)nc21] | 186 |
| 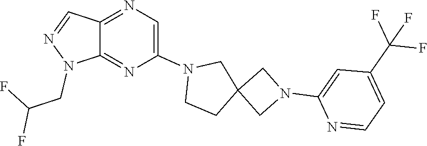 [SMILES: FC(F)Cn1ncc2ncc(N3CCC4(CN(c5cc(C(F)(F)F)ccn5)C4)C3)nc21] | 187 |
| 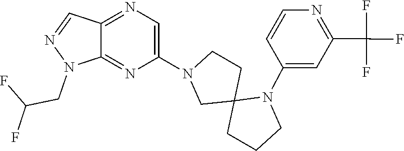 [SMILES: FC(F)Cn1ncc2ncc(N3CCC4(CCCN4c4ccnc(C(F)(F)F)c4)C3)nc21] | 188 |
| 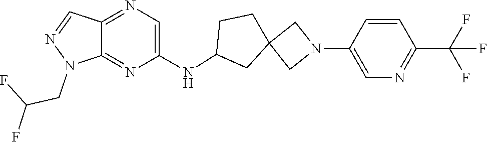 [SMILES: FC(F)Cn1ncc2ncc(NC3CCC4(C3)CN(c3ccc(C(F)(F)F)nc3)C4)nc21] | 189 |
| 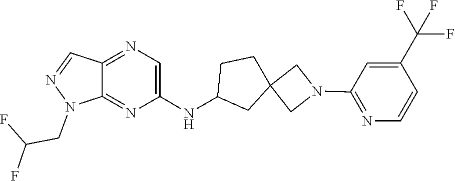 [SMILES: FC(F)Cn1ncc2ncc(NC3CCC4(C3)CN(c3cc(C(F)(F)F)ccn3)C4)nc21] | 190 |
| 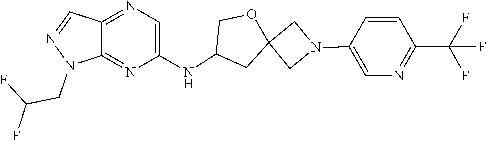 [SMILES: FC(F)Cn1ncc2ncc(NC3COC4(C3)CN(c3ccc(C(F)(F)F)nc3)C4)nc21] | 191 |
| 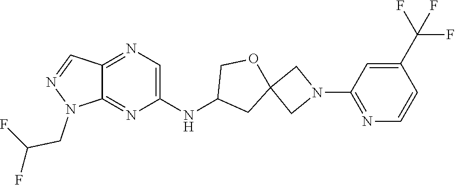 [SMILES: FC(F)Cn1ncc2ncc(NC3COC4(C3)CN(c3cc(C(F)(F)F)ccn3)C4)nc21] | 192 |
| 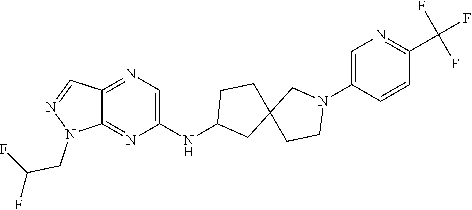 [SMILES: FC(F)Cn1ncc2ncc(NC3CCC4(CCN(c5ccc(C(F)(F)F)nc5)C4)C3)nc21] | 193 |
| 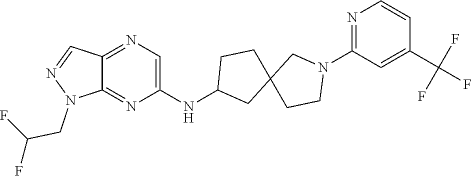 [SMILES: FC(F)Cn1ncc2ncc(NC3CCC4(CCN(c5cc(C(F)(F)F)ccn5)C4)C3)nc21] | 194 |
| 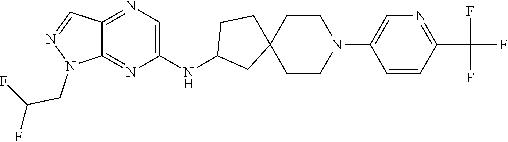 [SMILES: FC(F)Cn1ncc2ncc(NC3CCC4(CCN(c5ccc(C(F)(F)F)nc5)CC4)C3)nc21] | 195 |
| 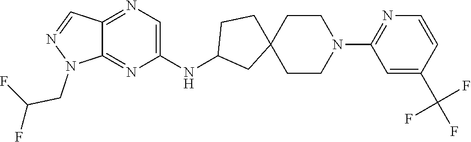 [SMILES: FC(F)Cn1ncc2ncc(NC3CCC4(CCN(c5cc(C(F)(F)F)ccn5)CC4)C3)nc21] | 196 |
| 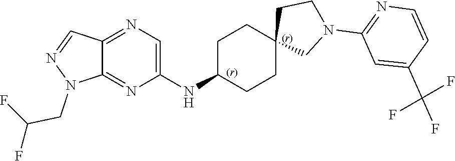 [SMILES: FC(F)Cn1ncc2ncc(N[C@H]3CC[C@@]4(CCN(c5cc(C(F)(F)F)ccn5)C4)CC3)nc21] | 197 |
| 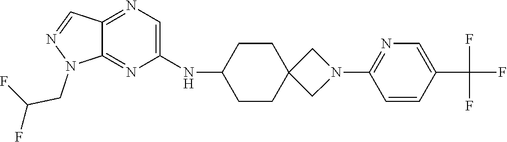 [SMILES: FC(F)Cn1ncc2ncc(NC3CCC4(CC3)CN(c3ccc(C(F)(F)F)cn3)C4)nc21] | 198 |
| 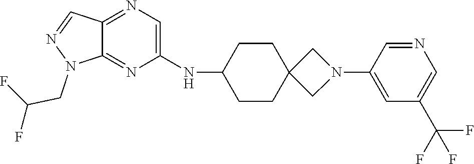 [SMILES: FC(F)Cn1ncc2ncc(NC3CCC4(CC3)CN(c3cncc(C(F)(F)F)c3)C4)nc21] | 199 |
| 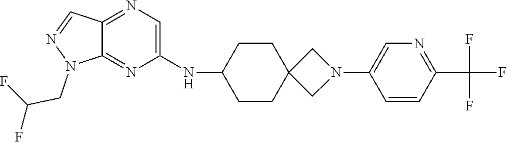 [SMILES: FC(F)Cn1ncc2ncc(NC3CCC4(CC3)CN(c3ccc(C(F)(F)F)nc3)C4)nc21] | 200 |
| 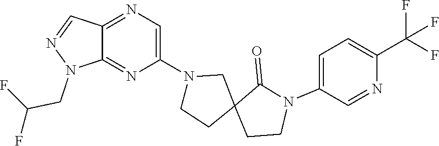 [SMILES: O=C1N(c2ccc(C(F)(F)F)nc2)CCC12CCN(c1cnc3cnn(CC(F)F)c3n1)C2] | 201 |
| 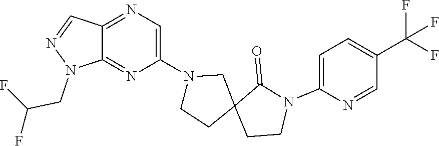 [SMILES: O=C1N(c2ccc(C(F)(F)F)cn2)CCC12CCN(c1cnc3cnn(CC(F)F)c3n1)C2] | 202 |
| 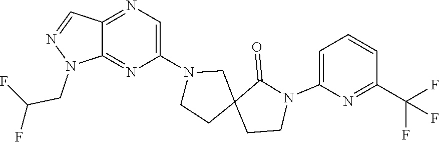 [SMILES: O=C1N(c2cccc(C(F)(F)F)n2)CCC12CCN(c1cnc3cnn(CC(F)F)c3n1)C2] | 203 |
| 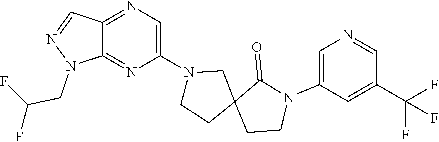 [SMILES: O=C1N(c2cncc(C(F)(F)F)c2)CCC12CCN(c1cnc3cnn(CC(F)F)c3n1)C2] | 204 |
| 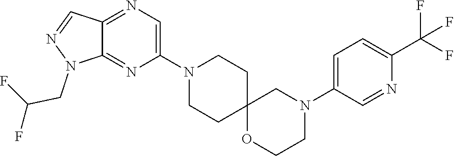 [SMILES: FC(F)Cn1ncc2ncc(N3CCC4(CC3)CN(c3ccc(C(F)(F)F)nc3)CCO4)nc21] | 205 |
| 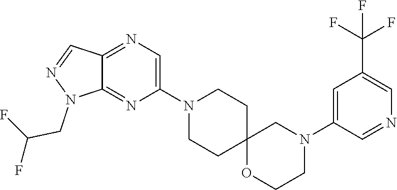 [SMILES: FC(F)Cn1ncc2ncc(N3CCC4(CC3)CN(c3cncc(C(F)(F)F)c3)CCO4)nc21] | 206 |
| 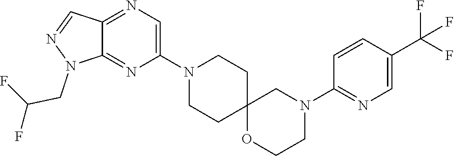 [SMILES: FC(F)Cn1ncc2ncc(N3CCC4(CC3)CN(c3ccc(C(F)(F)F)cn3)CCO4)nc21] | 207 |
| 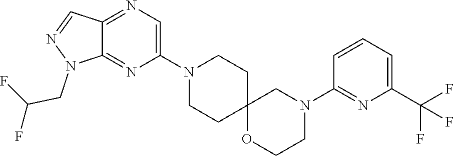 [SMILES: FC(F)Cn1ncc2ncc(N3CCC4(CC3)CN(c3cccc(C(F)(F)F)n3)CCO4)nc21] | 208 |
| 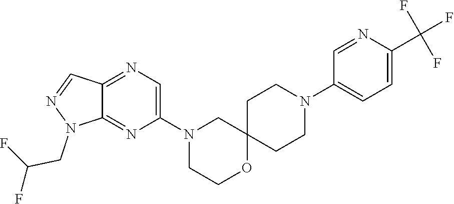 [SMILES: FC(F)Cn1ncc2ncc(N3CCOC4(CCN(c5ccc(C(F)(F)F)nc5)CC4)C3)nc21] | 209 |
| 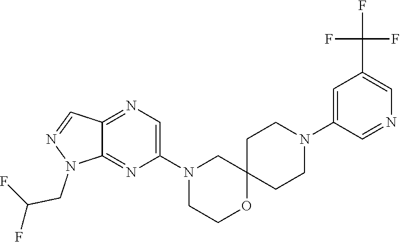 [SMILES: FC(F)Cn1ncc2ncc(N3CCOC4(CCN(c5cncc(C(F)(F)F)c5)CC4)C3)nc21] | 210 |
| 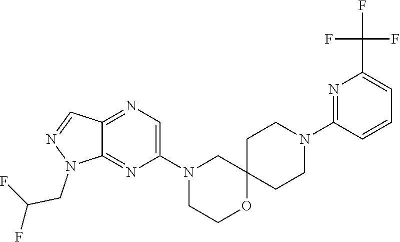 [SMILES: FC(F)Cn1ncc2ncc(N3CCOC4(CCN(c5cccc(C(F)(F)F)n5)CC4)C3)nc21] | 211 |
| 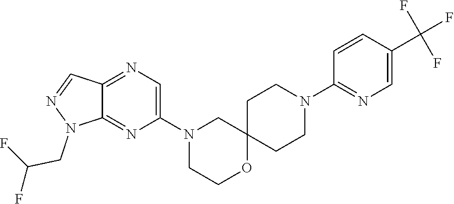 [SMILES: FC(F)Cn1ncc2ncc(N3CCOC4(CCN(c5ccc(C(F)(F)F)cn5)CC4)C3)nc21] | 212 |
| 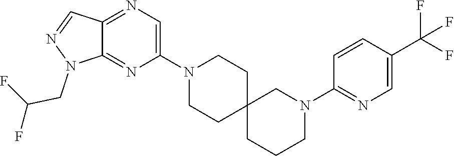 [SMILES: FC(F)Cn1ncc2ncc(N3CCC4(CCCN(c5ccc(C(F)(F)F)cn5)C4)CC3)nc21] | 213 |
| 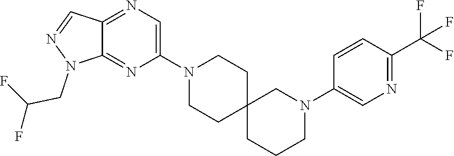 [SMILES: FC(F)Cn1ncc2ncc(N3CCC4(CCCN(c5ccc(C(F)(F)F)nc5)C4)CC3)nc21] | 214 |
| 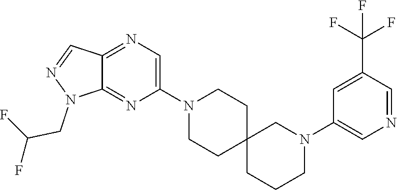 [SMILES: FC(F)Cn1ncc2ncc(N3CCC4(CCCN(c5cncc(C(F)(F)F)c5)C4)CC3)nc21] | 215 |
| 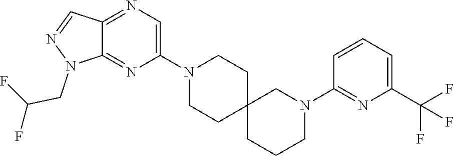 [SMILES: FC(F)Cn1ncc2ncc(N3CCC4(CCCN(c5cccc(C(F)(F)F)n5)C4)CC3)nc21] | 216 |
| 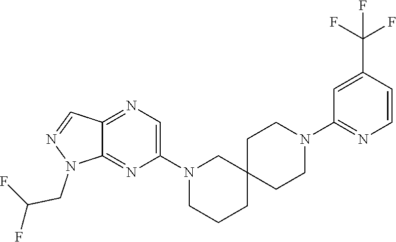 [SMILES: FC(F)Cn1ncc2ncc(N3CCCC4(CCN(c5cc(C(F)(F)F)ccn5)CC4)C3)nc21] | 217 |
| 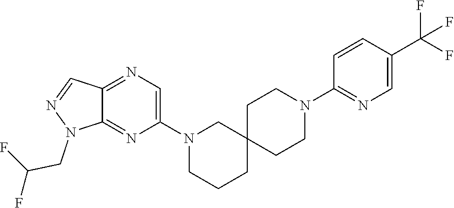 [SMILES: FC(F)Cn1ncc2ncc(N3CCCC4(CCN(c5ccc(C(F)(F)F)cn5)CC4)C3)nc21] | 218 |
| 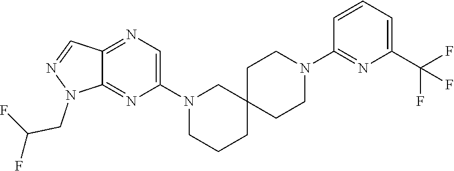 [SMILES: FC(F)Cn1ncc2ncc(N3CCCC4(CCN(c5cccc(C(F)(F)F)n5)CC4)C3)nc21] | 219 |
| 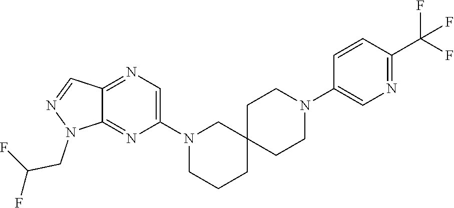 [SMILES: FC(F)Cn1ncc2ncc(N3CCCC4(CCN(c5ccc(C(F)(F)F)nc5)CC4)C3)nc21] | 220 |
| 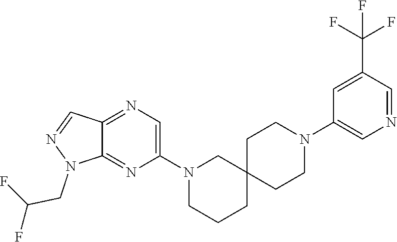 [SMILES: FC(F)Cn1ncc2ncc(N3CCCC4(CCN(c5cncc(C(F)(F)F)c5)CC4)C3)nc21] | 221 |
| 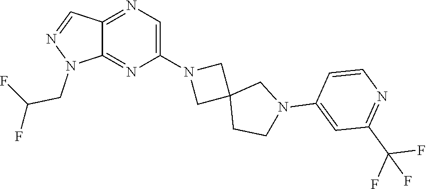 [SMILES: FC(F)Cn1ncc2ncc(N3CC4(CCN(c5ccnc(C(F)(F)F)c5)C4)C3)nc21] | 222 |
| 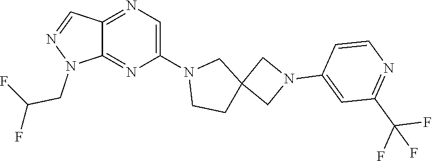 [SMILES: FC(F)Cn1ncc2ncc(N3CCC4(CN(c5ccnc(C(F)(F)F)c5)C4)C3)nc21] | 223 |
| 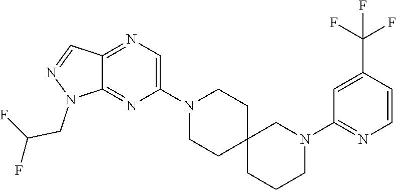 [SMILES: FC(F)Cn1ncc2ncc(N3CCC4(CCCN(c5cc(C(F)(F)F)ccn5)C4)CC3)nc21] | 224 |
| 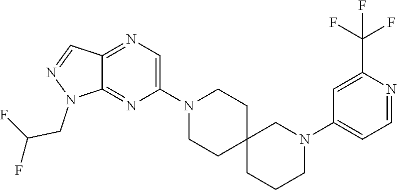 [SMILES: FC(F)Cn1ncc2ncc(N3CCC4(CCCN(c5ccnc(C(F)(F)F)c5)C4)CC3)nc21] | 225 |
| 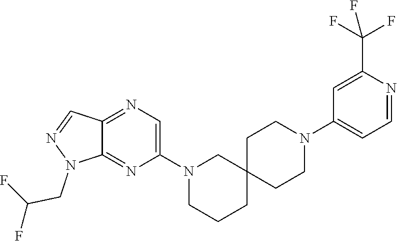 [SMILES: FC(F)Cn1ncc2ncc(N3CCCC4(CCN(c5ccnc(C(F)(F)F)c5)CC4)C3)nc21] | 226 |
| 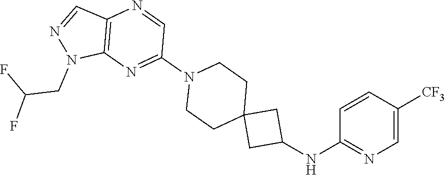 [SMILES: FC(F)Cn1ncc2ncc(N3CCC4(CC3)CC(Nc3ccc(C(F)(F)F)cn3)C4)nc21] | 227 |
| 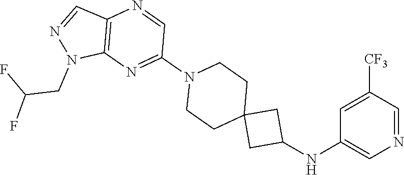 [SMILES: FC(F)Cn1ncc2ncc(N3CCC4(CC3)CC(Nc3cncc(C(F)(F)F)c3)C4)nc21] | 228 |
| 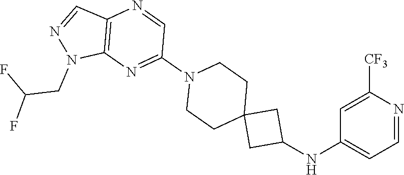 [SMILES: FC(F)Cn1ncc2ncc(N3CCC4(CC3)CC(Nc3ccnc(C(F)(F)F)c3)C4)nc21] | 229 |
| 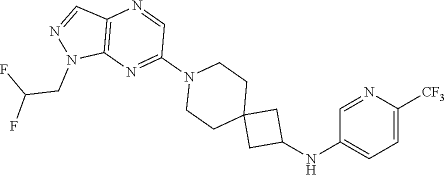 [SMILES: FC(F)Cn1ncc2ncc(N3CCC4(CC3)CC(Nc3ccc(C(F)(F)F)nc3)C4)nc21] | 230 |
| 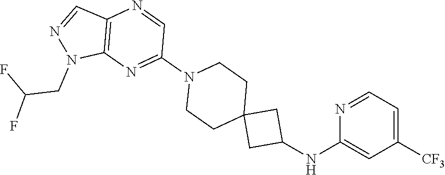 [SMILES: FC(F)Cn1ncc2ncc(N3CCC4(CC3)CC(Nc3cc(C(F)(F)F)ccn3)C4)nc21] | 231 |
| 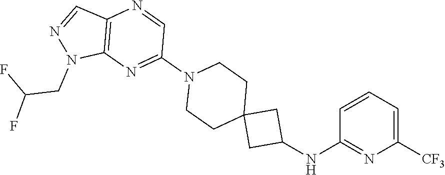 [SMILES: FC(F)Cn1ncc2ncc(N3CCC4(CC3)CC(Nc3cccc(C(F)(F)F)n3)C4)nc21] | 232 |
| 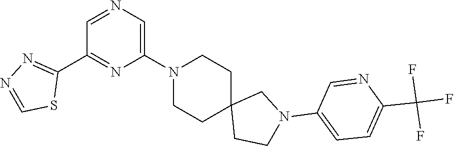 [SMILES: FC(F)(F)c1ccc(N2CCC3(CCN(c4cncc(-c5nncs5)n4)CC3)C2)cn1] | 233 |
| 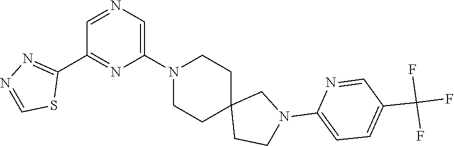 [SMILES: FC(F)(F)c1ccc(N2CCC3(CCN(c4cncc(-c5nncs5)n4)CC3)C2)nc1] | 234 |
| 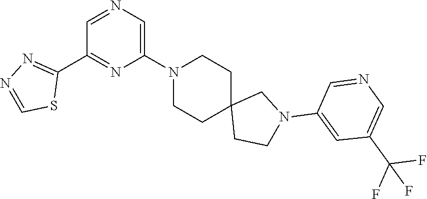 [SMILES: FC(F)(F)c1cncc(N2CCC3(CCN(c4cncc(-c5nncs5)n4)CC3)C2)c1] | 235 |
| 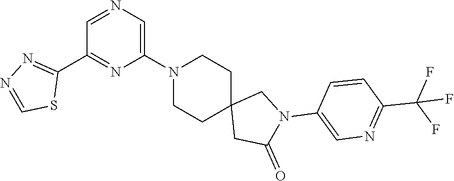 [SMILES: O=C1CC2(CCN(c3cncc(-c4nncs4)n3)CC2)CN1c1ccc(C(F)(F)F)nc1] | 236 |
| 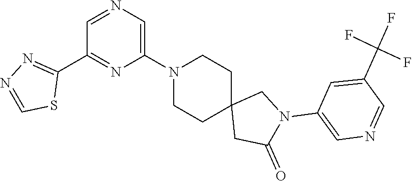 [SMILES: O=C1CC2(CCN(c3cncc(-c4nncs4)n3)CC2)CN1c1cncc(C(F)(F)F)c1] | 237 |
| 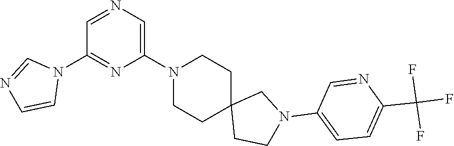 [SMILES: FC(F)(F)c1ccc(N2CCC3(CCN(c4cncc(-n5ccnc5)n4)CC3)C2)cn1] | 238 |
| 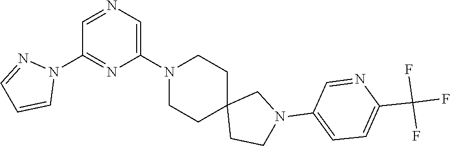 [SMILES: FC(F)(F)c1ccc(N2CCC3(CCN(c4cncc(-n5cccn5)n4)CC3)C2)cn1] | 239 |
| 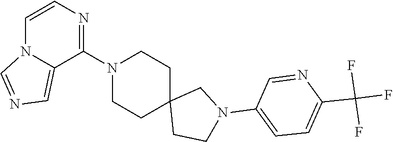 [SMILES: FC(F)(F)c1ccc(N2CCC3(CCN(c4nccn5cncc45)CC3)C2)cn1] | 240 |
| 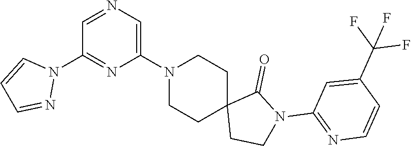 [SMILES: O=C1N(c2cc(C(F)(F)F)ccn2)CCC12CCN(c1cncc(-n3cccn3)n1)CC2] | 241 |
| 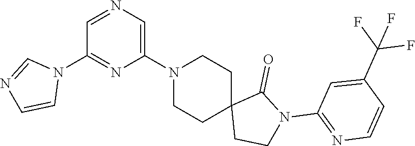 [SMILES: O=C1N(c2cc(C(F)(F)F)ccn2)CCC12CCN(c1cncc(-n3ccnc3)n1)CC2] | 242 |
| 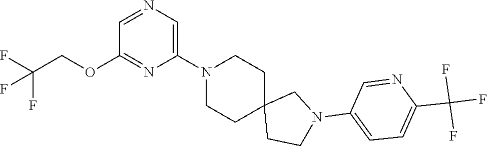 [SMILES: FC(F)(F)COc1cncc(N2CCC3(CCN(c4ccc(C(F)(F)F)nc4)C3)CC2)n1] | 243 |
| 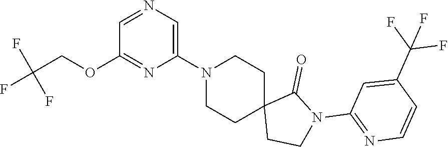 [SMILES: O=C1N(c2cc(C(F)(F)F)ccn2)CCC12CCN(c1cncc(OCC(F)(F)F)n1)CC2] | 244 |
| 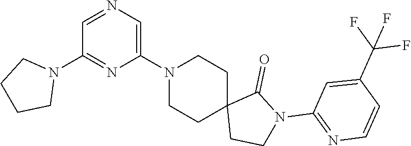 [SMILES: O=C1N(c2cc(C(F)(F)F)ccn2)CCC12CCN(c1cncc(N3CCCC3)n1)CC2] | 245 |
| 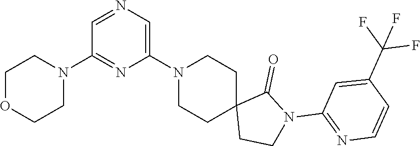 [SMILES: O=C1N(c2cc(C(F)(F)F)ccn2)CCC12CCN(c1cncc(N3CCOCC3)n1)CC2] | 246 |
| 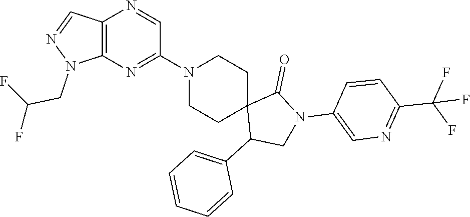 [SMILES: O=C1N(c2ccc(C(F)(F)F)nc2)CC(c2ccccc2)C12CCN(c1cnc3cnn(CC(F)F)c3n1)CC2] | 247 |
| 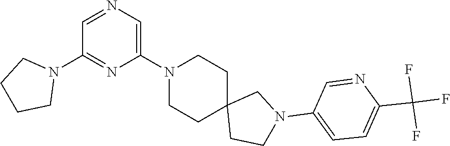 [SMILES: FC(F)(F)c1ccc(N2CCC3(CCN(c4cncc(N5CCCC5)n4)CC3)C2)cn1] | 248 |
| 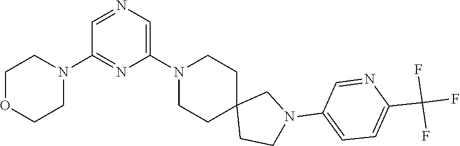 [SMILES: FC(F)(F)c1ccc(N2CCC3(CCN(c4cncc(N5CCOCC5)n4)CC3)C2)cn1] | 249 |
| 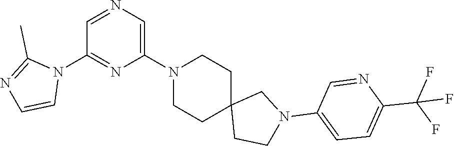 [SMILES: Cc1nccn1-c1cncc(N2CCC3(CCN(c4ccc(C(F)(F)F)nc4)C3)CC2)n1] | 250 |
| 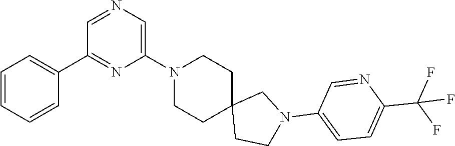 [SMILES: FC(F)(F)c1ccc(N2CCC3(CCN(c4cncc(-c5ccccc5)n4)CC3)C2)cn1] | 251 |
| 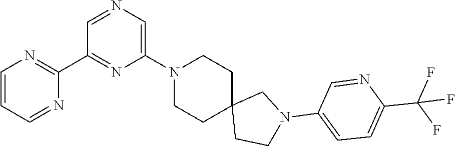 [SMILES: FC(F)(F)c1ccc(N2CCC3(CCN(c4cncc(-c5ncccn5)n4)CC3)C2)cn1] | 252 |
| 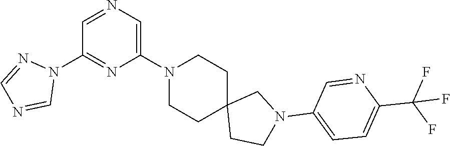 [SMILES: FC(F)(F)c1ccc(N2CCC3(CCN(c4cncc(-n5cncn5)n4)CC3)C2)cn1] | 253 |
| 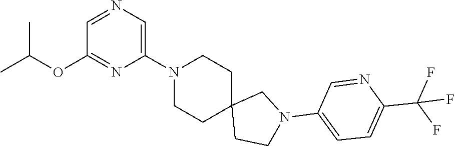 [SMILES: CC(C)Oc1cncc(N2CCC3(CCN(c4ccc(C(F)(F)F)nc4)C3)CC2)n1] | 254 |
| 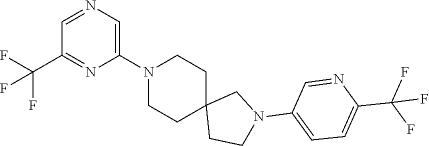 [SMILES: FC(F)(F)c1ccc(N2CCC3(CCN(c4cncc(C(F)(F)F)n4)CC3)C2)cn1] | 255 |
| 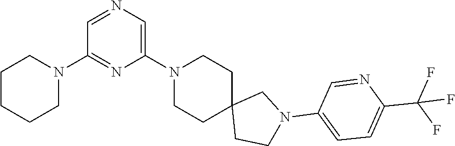 [SMILES: FC(F)(F)c1ccc(N2CCC3(CCN(c4cncc(N5CCCCC5)n4)CC3)C2)cn1] | 256 |
| 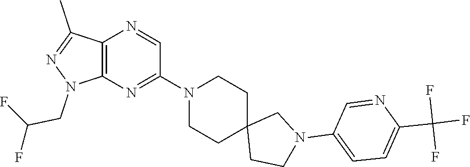 [SMILES: Cc1nn(CC(F)F)c2nc(N3CCC4(CCN(c5ccc(C(F)(F)F)nc5)C4)CC3)cnc12] | 257 |
| 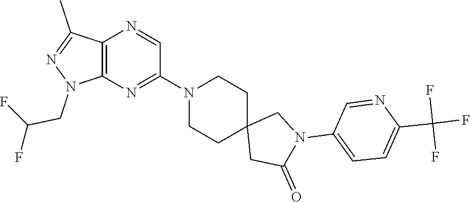 [SMILES: Cc1nn(CC(F)F)c2nc(N3CCC4(CC3)CC(=O)N(c3ccc(C(F)(F)F)nc3)C4)cnc12] | 258 |
| 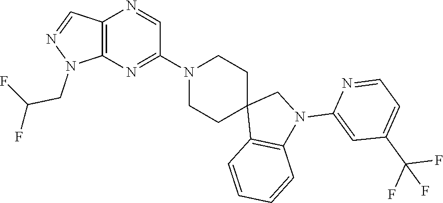 [SMILES: FC(F)Cn1ncc2ncc(N3CCC4(CC3)CN(c3cc(C(F)(F)F)ccn3)c3ccccc34)nc21] | 259 |
| 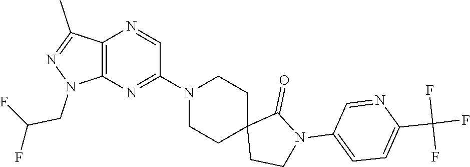 [SMILES: Cc1nn(CC(F)F)c2nc(N3CCC4(CC3)CCN(c3ccc(C(F)(F)F)nc3)C4=O)cnc12] | 260 |
| 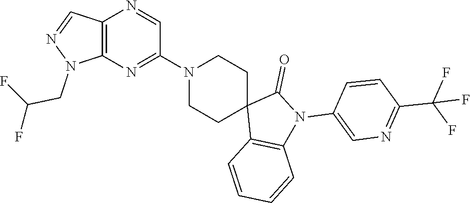 [SMILES: O=C1N(c2ccc(C(F)(F)F)nc2)c2ccccc2C12CCN(c1cnc3cnn(CC(F)F)c3n1)CC2] | 261 |
| 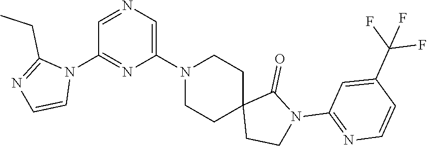 [SMILES: CCc1nccn1-c1cncc(N2CCC3(CC2)CCN(c2cc(C(F)(F)F)ccn2)C3=O)n1] | 262 |
| 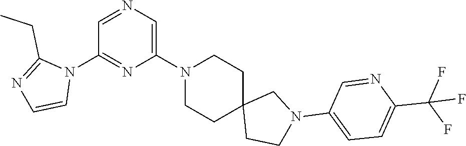 [SMILES: CCc1nccn1-c1cncc(N2CCC3(CCN(c4ccc(C(F)(F)F)nc4)C3)CC2)n1] | 263 |
| 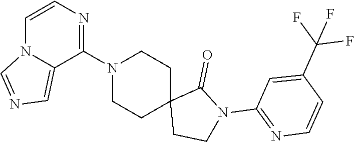 [SMILES: O=C1N(c2cc(C(F)(F)F)ccn2)CCC12CCN(c1nccn3cncc13)CC2] | 264 |
| 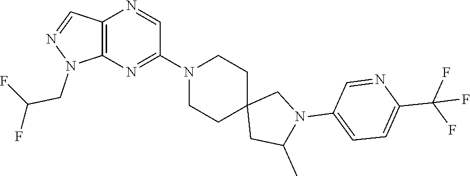 [SMILES: CC1CC2(CCN(c3cnc4cnn(CC(F)F)c4n3)CC2)CN1c1ccc(C(F)(F)F)nc1] | 265 |
| 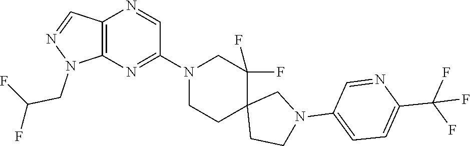 [SMILES: FC(F)Cn1ncc2ncc(N3CCC4(CCN(c5ccc(C(F)(F)F)nc5)C4)C(F)(F)C3)nc21] | 266 |
| 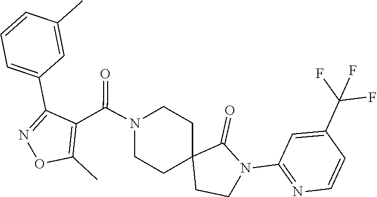 [SMILES: Cc1cccc(-c2noc(C)c2C(=O)N2CCC3(CC2)CCN(c2cc(C(F)(F)F)ccn2)C3=O)c1] | 267 |
| 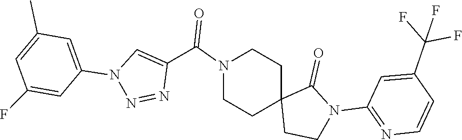 [SMILES: Cc1cc(F)cc(-n2cc(C(=O)N3CCC4(CC3)CCN(c3cc(C(F)(F)F)ccn3)C4=O)nn2)c1] | 268 |
| 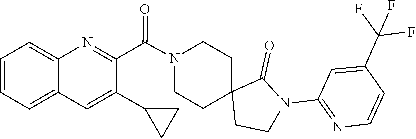 [SMILES: O=C(c1nc2ccccc2cc1C1CC1)N1CCC2(CC1)CCN(c1cc(C(F)(F)F)ccn1)C2=O] | 269 |
| 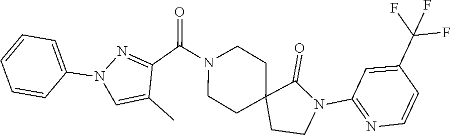 [SMILES: Cc1cn(-c2ccccc2)nc1C(=O)N1CCC2(CC1)CCN(c1cc(C(F)(F)F)ccn1)C2=O] | 270 |
| 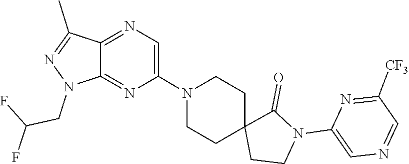 [SMILES: Cc1nn(CC(F)F)c2nc(N3CCC4(CC3)CCN(c3cncc(C(F)(F)F)n3)C4=O)cnc12] | 271 |
| 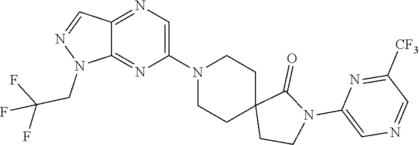 [SMILES: O=C1N(c2cncc(C(F)(F)F)n2)CCC12CCN(c1cnc3cnn(CC(F)(F)F)c3n1)CC2] | 272 |
| 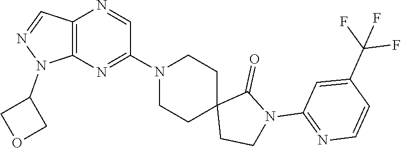 [SMILES: O=C1N(c2cc(C(F)(F)F)ccn2)CCC12CCN(c1cnc3cnn(C4COC4)c3n1)CC2] | 273 |
| 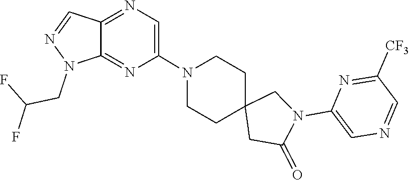 [SMILES: O=C1CC2(CCN(c3cnc4cnn(CC(F)F)c4n3)CC2)CN1c1cncc(C(F)(F)F)n1] | 274 |
| 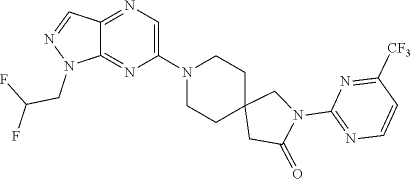 [SMILES: O=C1CC2(CCN(c3cnc4cnn(CC(F)F)c4n3)CC2)CN1c1nccc(C(F)(F)F)n1] | 275 |
| 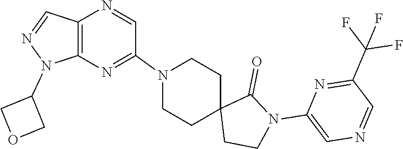 [SMILES: O=C1N(c2cncc(C(F)(F)F)n2)CCC12CCN(c1cnc3cnn(C4COC4)c3n1)CC2] | 276 |
| 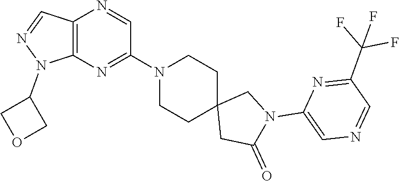 [SMILES: O=C1CC2(CCN(c3cnc4cnn(C5COC5)c4n3)CC2)CN1c1cncc(C(F)(F)F)n1] | 277 |
| 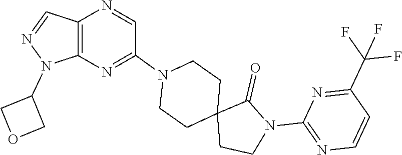 [SMILES: O=C1N(c2nccc(C(F)(F)F)n2)CCC12CCN(c1cnc3cnn(C4COC4)c3n1)CC2] | 278 |
| 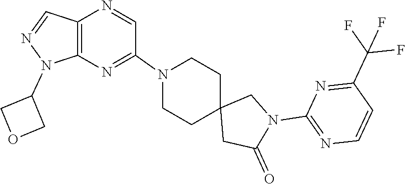 [SMILES: O=C1CC2(CCN(c3cnc4cnn(C5COC5)c4n3)CC2)CN1c1nccc(C(F)(F)F)n1] | 279 |
| 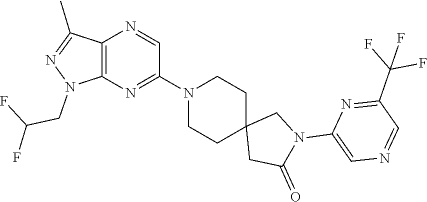 [SMILES: Cc1nn(CC(F)F)c2nc(N3CCC4(CC3)CC(=O)N(c3cncc(C(F)(F)F)n3)C4)cnc12] | 280 |
| 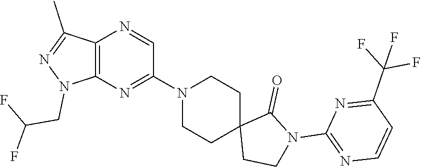 [SMILES: Cc1nn(CC(F)F)c2nc(N3CCC4(CC3)CCN(c3nccc(C(F)(F)F)n3)C4=O)cnc12] | 281 |
| 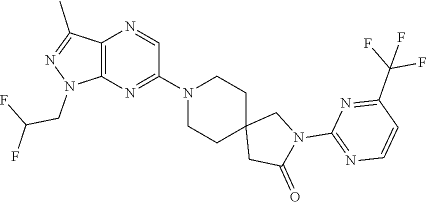 [SMILES: Cc1nn(CC(F)F)c2nc(N3CCC4(CC3)CC(=O)N(c3nccc(C(F)(F)F)n3)C4)cnc12] | 282 |
| 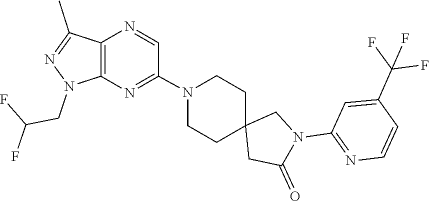 [SMILES: Cc1nn(CC(F)F)c2nc(N3CCC4(CC3)CC(=O)N(c3cc(C(F)(F)F)ccn3)C4)cnc12] | 283 |
| 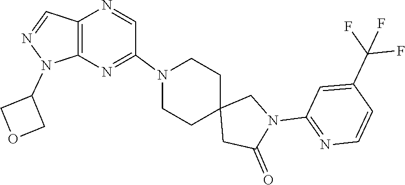 [SMILES: O=C1CC2(CCN(c3cnc4cnn(C5COC5)c4n3)CC2)CN1c1cc(C(F)(F)F)ccn1] | 284 |
| 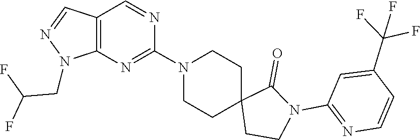 [SMILES: O=C1N(c2cc(C(F)(F)F)ccn2)CCC12CCN(c1ncc3cnn(CC(F)F)c3n1)CC2] | 285 |
| 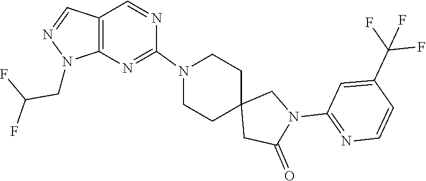 [SMILES: O=C1CC2(CCN(c3ncc4cnn(CC(F)F)c4n3)CC2)CN1c1cc(C(F)(F)F)ccn1] | 286 |
| 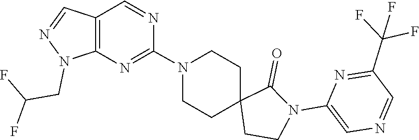 [SMILES: O=C1N(c2cncc(C(F)(F)F)n2)CCC12CCN(c1ncc3cnn(CC(F)F)c3n1)CC2] | 287 |
| 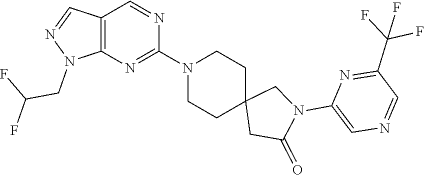 [SMILES: O=C1CC2(CCN(c3ncc4cnn(CC(F)F)c4n3)CC2)CN1c1cncc(C(F)(F)F)n1] | 288 |
| 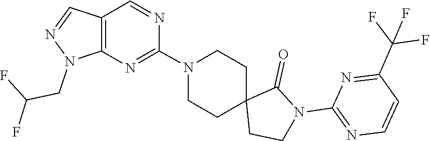 [SMILES: O=C1N(c2nccc(C(F)(F)F)n2)CCC12CCN(c1ncc3cnn(CC(F)F)c3n1)CC2] | 289 |
| 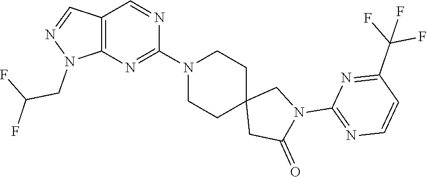 [SMILES: O=C1CC2(CCN(c3ncc4cnn(CC(F)F)c4n3)CC2)CN1c1nccc(C(F)(F)F)n1] | 290 |
| 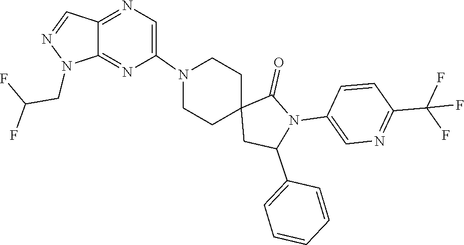 [SMILES: O=C1N(c2ccc(C(F)(F)F)nc2)C(c2ccccc2)CC12CCN(c1cnc3cnn(CC(F)F)c3n1)CC2] | 291 |
| 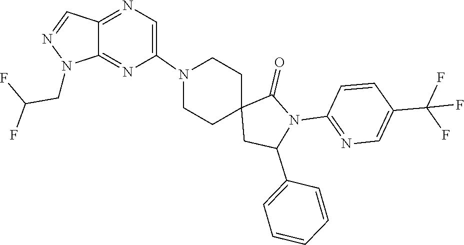 [SMILES: O=C1N(c2ccc(C(F)(F)F)cn2)C(c2ccccc2)CC12CCN(c1cnc3cnn(CC(F)F)c3n1)CC2] | 292 |
| 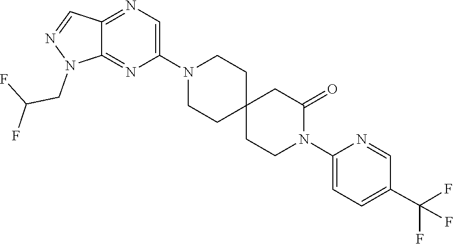 [SMILES: O=C1CC2(CCN(c3cnc4cnn(CC(F)F)c4n3)CC2)CCN1c1ccc(C(F)(F)F)cn1] | 293 |
| 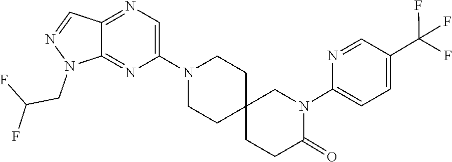 [SMILES: O=C1CCC2(CCN(c3cnc4cnn(CC(F)F)c4n3)CC2)CN1c1ccc(C(F)(F)F)cn1] | 294 |
| 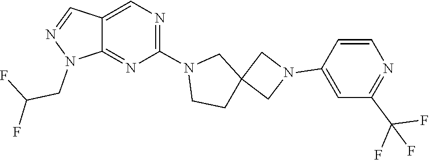 [SMILES: FC(F)Cn1ncc2cnc(N3CCC4(CN(c5ccnc(C(F)(F)F)c5)C4)C3)nc21] | 295 |
| 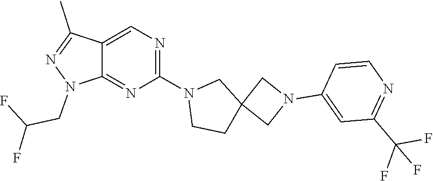 [SMILES: Cc1nn(CC(F)F)c2nc(N3CCC4(CN(c5ccnc(C(F)(F)F)c5)C4)C3)ncc12] | 296 |
| 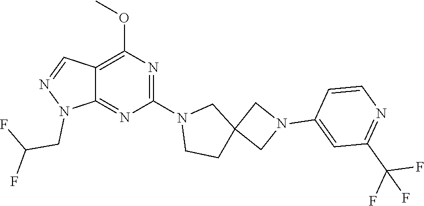 [SMILES: COc1nc(N2CCC3(CN(c4ccnc(C(F)(F)F)c4)C3)C2)nc2c1cnn2CC(F)F] | 297 |
| 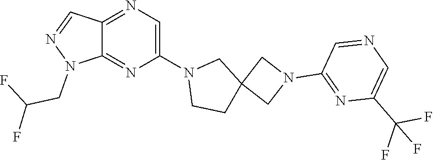 [SMILES: FC(F)Cn1ncc2ncc(N3CCC4(CN(c5cncc(C(F)(F)F)n5)C4)C3)nc21] | 298 |
| 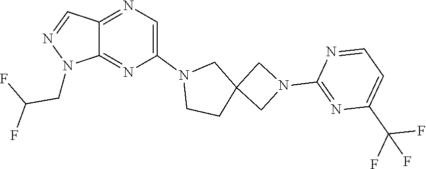 [SMILES: FC(F)Cn1ncc2ncc(N3CCC4(C3)CN(c3nccc(C(F)(F)F)n3)C4)nc21] | 299 |
| 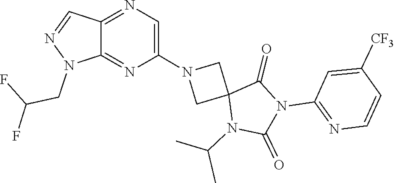 [SMILES: CC(C)N1C(=O)N(c2cc(C(F)(F)F)ccn2)C(=O)C12CN(c1cnc3cnn(CC(F)F)c3n1)C2] | 300 |
| 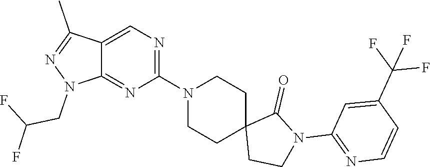 [SMILES: Cc1nn(CC(F)F)c2nc(N3CCC4(CC3)CCN(c3cc(C(F)(F)F)ccn3)C4=O)ncc12] | 301 |
| 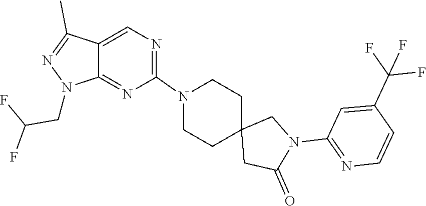 [SMILES: Cc1nn(CC(F)F)c2nc(N3CCC4(CC3)CC(=O)N(c3cc(C(F)(F)F)ccn3)C4)ncc12] | 302 |
| 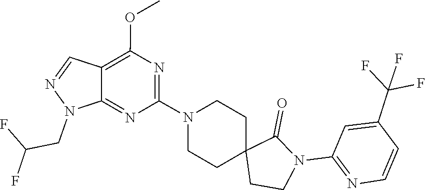 [SMILES: COc1nc(N2CCC3(CC2)CCN(c2cc(C(F)(F)F)ccn2)C3=O)nc2c1cnn2CC(F)F] | 303 |
| 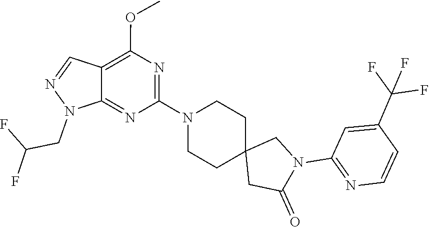 [SMILES: COc1nc(N2CCC3(CC2)CC(=O)N(c2cc(C(F)(F)F)ccn2)C3)nc2c1cnn2CC(F)F] | 304 |
| 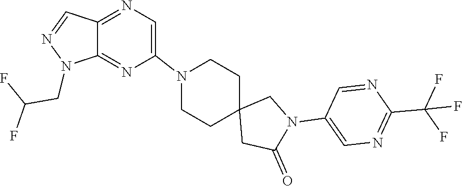 [SMILES: O=C1CC2(CCN(c3cnc4cnn(CC(F)F)c4n3)CC2)CN1c1cnc(C(F)(F)F)nc1] | 305 |
| 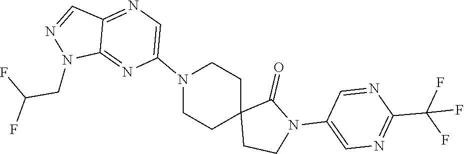 [SMILES: O=C1N(c2cnc(C(F)(F)F)nc2)CCC12CCN(c1cnc3cnn(CC(F)F)c3n1)CC2] | 306 |
| 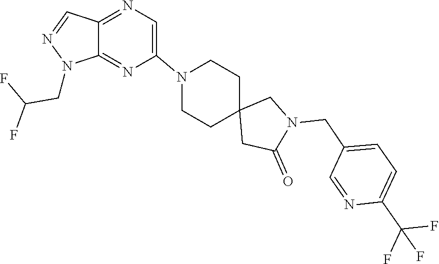 [SMILES: O=C1CC2(CCN(c3cnc4cnn(CC(F)F)c4n3)CC2)CN1Cc1ccc(C(F)(F)F)nc1] | 307 |
| 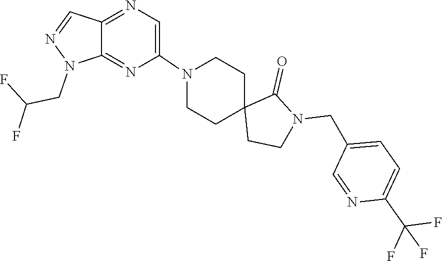 [SMILES: O=C1N(Cc2ccc(C(F)(F)F)nc2)CCC12CCN(c1cnc3cnn(CC(F)F)c3n1)CC2] | 308 |
| 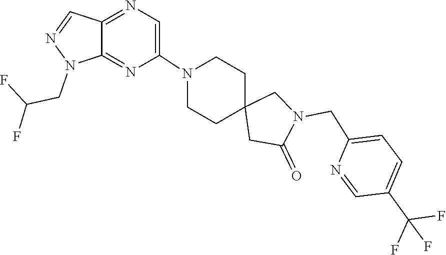 [SMILES: O=C1CC2(CCN(c3cnc4cnn(CC(F)F)c4n3)CC2)CN1Cc1ccc(C(F)(F)F)cn1] | 309 |
| 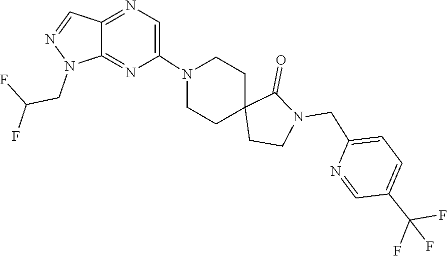 [SMILES: O=C1N(Cc2ccc(C(F)(F)F)cn2)CCC12CCN(c1cnc3cnn(CC(F)F)c3n1)CC2] | 310 |
| 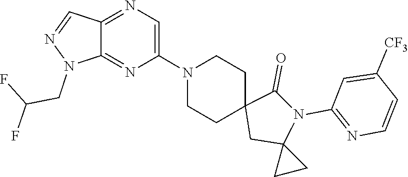 [SMILES: O=C1N(c2cc(C(F)(F)F)ccn2)C2(CC2)CC12CCN(c1cnc3cnn(CC(F)F)c3n1)CC2] | 311 |
| 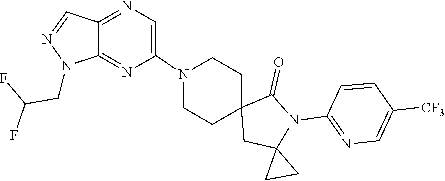 [SMILES: O=C1N(c2ccc(C(F)(F)F)cn2)C2(CC2)CC12CCN(c1cnc3cnn(CC(F)F)c3n1)CC2] | 312 |
| 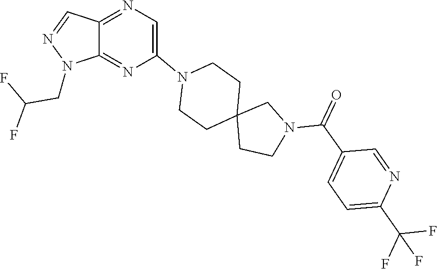 [SMILES: O=C(c1ccc(C(F)(F)F)nc1)N1CCC2(CCN(c3cnc4cnn(CC(F)F)c4n3)CC2)C1] | 313 |
| 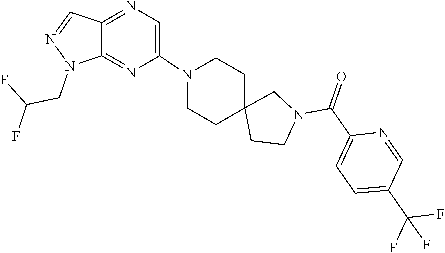 [SMILES: O=C(c1ccc(C(F)(F)F)cn1)N1CCC2(CCN(c3cnc4cnn(CC(F)F)c4n3)CC2)C1] | 314 |
| 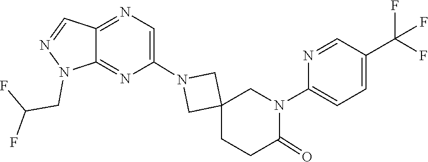 [SMILES: O=C1CCC2(CN(c3cnc4cnn(CC(F)F)c4n3)C2)CN1c1ccc(C(F)(F)F)cn1] | 315 |
| 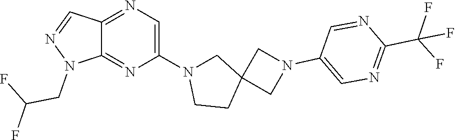 [SMILES: FC(F)Cn1ncc2ncc(N3CCC4(CN(c5cnc(C(F)(F)F)nc5)C4)C3)nc21] | 316 |
| 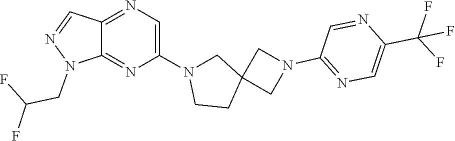 [SMILES: FC(F)Cn1ncc2ncc(N3CCC4(CN(c5cnc(C(F)(F)F)cn5)C4)C3)nc21] | 317 |
| 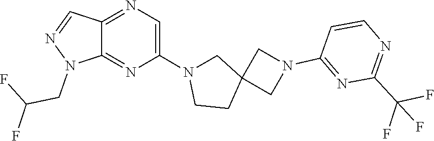 [SMILES: FC(F)Cn1ncc2ncc(N3CCC4(CN(c5ccnc(C(F)(F)F)n5)C4)C3)nc21] | 318 |
| 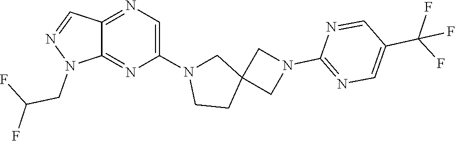 [SMILES: FC(F)Cn1ncc2ncc(N3CCC4(C3)CN(c3ncc(C(F)(F)F)cn3)C4)nc21] | 319 |
| 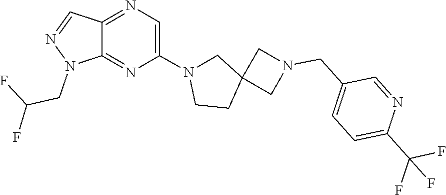 [SMILES: FC(F)Cn1ncc2ncc(N3CCC4(CN(Cc5ccc(C(F)(F)F)nc5)C4)C3)nc21] | 320 |
| 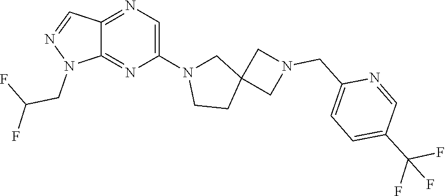 [SMILES: FC(F)Cn1ncc2ncc(N3CCC4(CN(Cc5ccc(C(F)(F)F)cn5)C4)C3)nc21] | 321 |
| 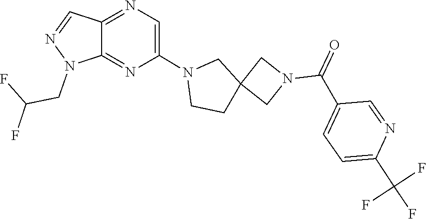 [SMILES: O=C(c1ccc(C(F)(F)F)nc1)N1CC2(CCN(c3cnc4cnn(CC(F)F)c4n3)C2)C1] | 322 |
| 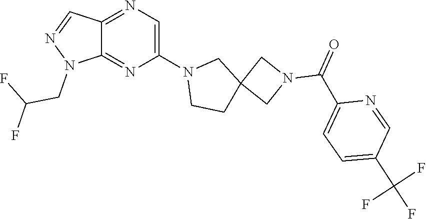 [SMILES: O=C(c1ccc(C(F)(F)F)cn1)N1CC2(CCN(c3cnc4cnn(CC(F)F)c4n3)C2)C1] | 323 |
| 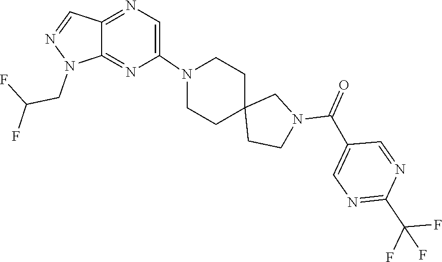 [SMILES: O=C(c1cnc(C(F)(F)F)nc1)N1CCC2(CCN(c3cnc4cnn(CC(F)F)c4n3)CC2)C1] | 324 |
| 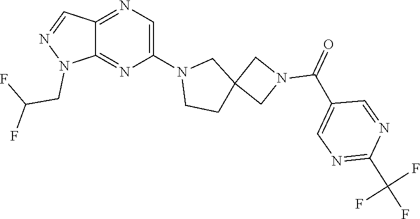 [SMILES: O=C(c1cnc(C(F)(F)F)nc1)N1CC2(CCN(c3cnc4cnn(CC(F)F)c4n3)C2)C1] | 325 |
| 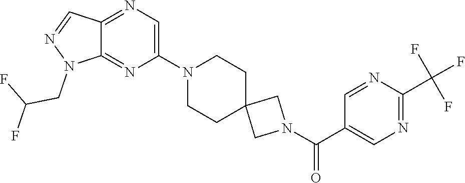 [SMILES: O=C(c1cnc(C(F)(F)F)nc1)N1CC2(CCN(c3cnc4cnn(CC(F)F)c4n3)CC2)C1] | 326 |
| 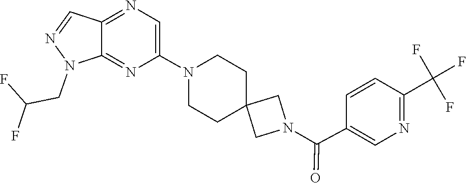 [SMILES: O=C(c1ccc(C(F)(F)F)nc1)N1CC2(CCN(c3cnc4cnn(CC(F)F)c4n3)CC2)C1] | 327 |
| 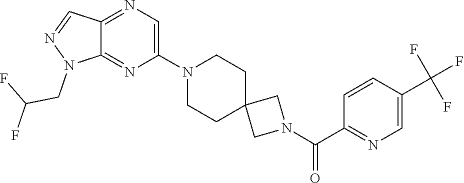 [SMILES: O=C(c1ccc(C(F)(F)F)cn1)N1CC2(CCN(c3cnc4cnn(CC(F)F)c4n3)CC2)C1] | 328 |
| 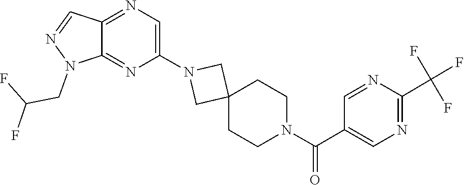 [SMILES: O=C(c1cnc(C(F)(F)F)nc1)N1CCC2(CC1)CN(c1cnc3cnn(CC(F)F)c3n1)C2] | 329 |
| 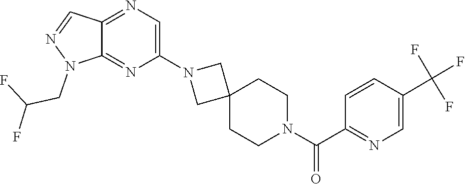 [SMILES: O=C(c1ccc(C(F)(F)F)cn1)N1CCC2(CC1)CN(c1cnc3cnn(CC(F)F)c3n1)C2] | 330 |
| 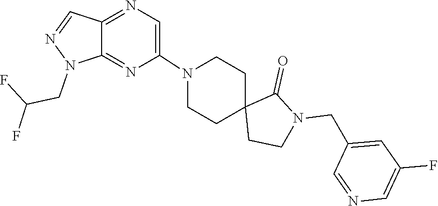 [SMILES: O=C1N(Cc2cncc(F)c2)CCC12CCN(c1cnc3cnn(CC(F)F)c3n1)CC2] | 331 |
| 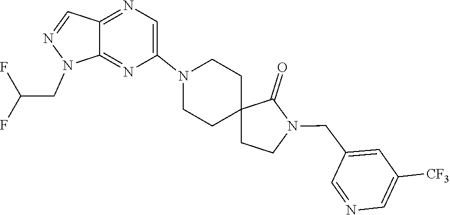 [SMILES: O=C1N(Cc2cncc(C(F)(F)F)c2)CCC12CCN(c1cnc3cnn(CC(F)F)c3n1)CC2] | 332 |
| 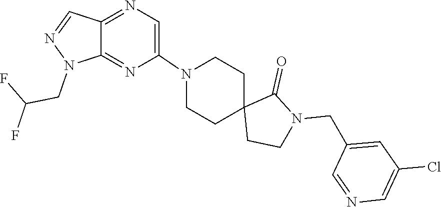 [SMILES: O=C1N(Cc2cncc(Cl)c2)CCC12CCN(c1cnc3cnn(CC(F)F)c3n1)CC2] | 333 |
| 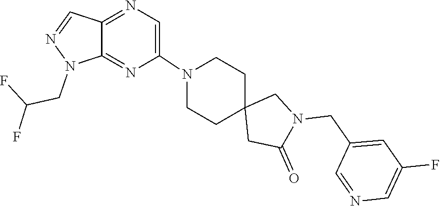 [SMILES: O=C1CC2(CCN(c3cnc4cnn(CC(F)F)c4n3)CC2)CN1Cc1cncc(F)c1] | 334 |
| 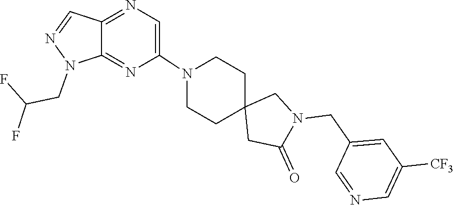 [SMILES: O=C1CC2(CCN(c3cnc4cnn(CC(F)F)c4n3)CC2)CN1Cc1cncc(C(F)(F)F)c1] | 335 |
| 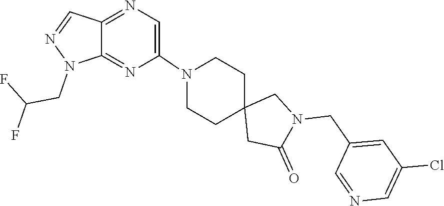 [SMILES: O=C1CC2(CCN(c3cnc4cnn(CC(F)F)c4n3)CC2)CN1Cc1cncc(Cl)c1] | 336 |
| 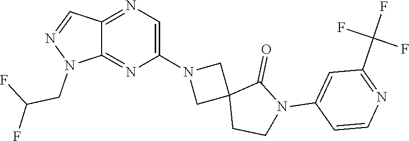 [SMILES: O=C1N(c2ccnc(C(F)(F)F)c2)CCC12CN(c1cnc3cnn(CC(F)F)c3n1)C2] | 337 |
| 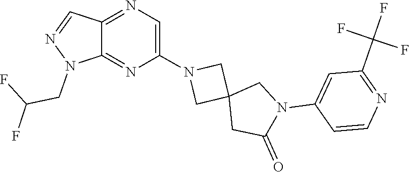 [SMILES: O=C1CC2(CN(c3cnc4cnn(CC(F)F)c4n3)C2)CN1c1ccnc(C(F)(F)F)c1] | 338 |
| 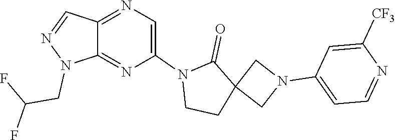 [SMILES: O=C1N(c2cnc3cnn(CC(F)F)c3n2)CCC12CN(c1ccnc(C(F)(F)F)c1)C2] | 339 |
| 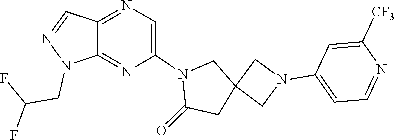 [SMILES: O=C1CC2(CN(c3ccnc(C(F)(F)F)c3)C2)CN1c1cnc2cnn(CC(F)F)c2n1] | 340 |
| 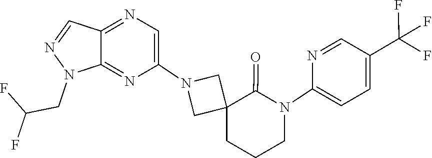 [SMILES: O=C1N(c2ccc(C(F)(F)F)cn2)CCCC12CN(c1cnc3cnn(CC(F)F)c3n1)C2] | 341 |
| 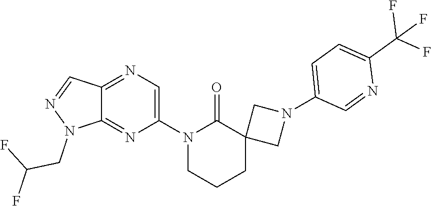 [SMILES: O=C1N(c2cnc3cnn(CC(F)F)c3n2)CCCC12CN(c1ccc(C(F)(F)F)nc1)C2] | 342 |
| 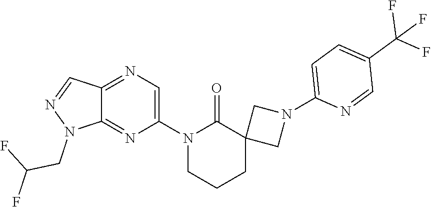 [SMILES: O=C1N(c2cnc3cnn(CC(F)F)c3n2)CCCC12CN(c1ccc(C(F)(F)F)cn1)C2] | 343 |
| 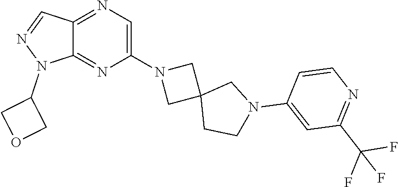 [SMILES: FC(F)(F)c1cc(N2CCC3(C2)CN(c2cnc4cnn(C5COC5)c4n2)C3)ccn1] | 344 |
| 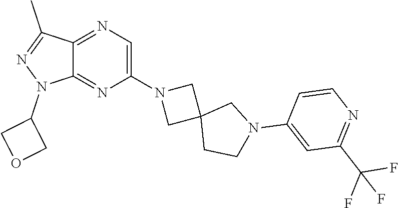 [SMILES: Cc1nn(C2COC2)c2nc(N3CC4(CCN(c5ccnc(C(F)(F)F)c5)C4)C3)cnc12] | 345 |
| 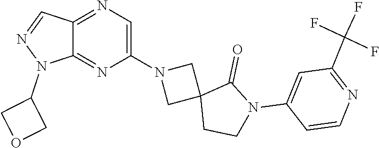 [SMILES: O=C1N(c2ccnc(C(F)(F)F)c2)CCC12CN(c1cnc3cnn(C4COC4)c3n1)C2] | 346 |
| 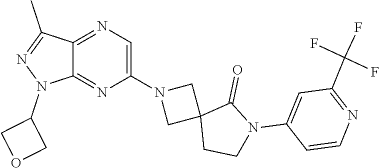 [SMILES: Cc1nn(C2COC2)c2nc(N3CC4(CCN(c5ccnc(C(F)(F)F)c5)C4=O)C3)cnc12] | 347 |
| 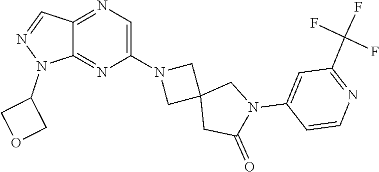 [SMILES: O=C1CC2(CN(c3cnc4cnn(C5COC5)c4n3)C2)CN1c1ccnc(C(F)(F)F)c1] | 348 |
| 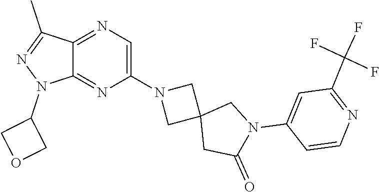 [SMILES: Cc1nn(C2COC2)c2nc(N3CC4(CC(=O)N(c5ccnc(C(F)(F)F)c5)C4)C3)cnc12] | 349 |
| 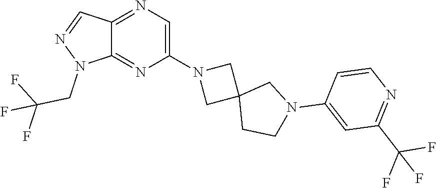 [SMILES: FC(F)(F)Cn1ncc2ncc(N3CC4(CCN(c5ccnc(C(F)(F)F)c5)C4)C3)nc21] | 350 |
| 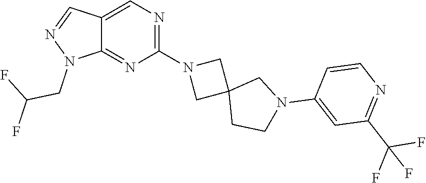 [SMILES: FC(F)Cn1ncc2cnc(N3CC4(CCN(c5ccnc(C(F)(F)F)c5)C4)C3)nc21] | 351 |
| 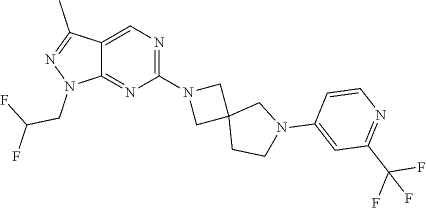 [SMILES: Cc1nn(CC(F)F)c2nc(N3CC4(CCN(c5ccnc(C(F)(F)F)c5)C4)C3)ncc12] | 352 |
| 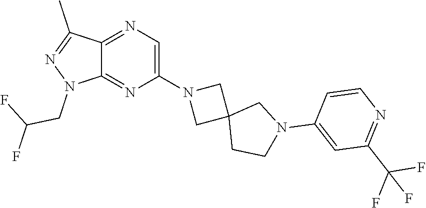 [SMILES: Cc1nn(CC(F)F)c2nc(N3CC4(CCN(c5ccnc(C(F)(F)F)c5)C4)C3)cnc12] | 353 |
| 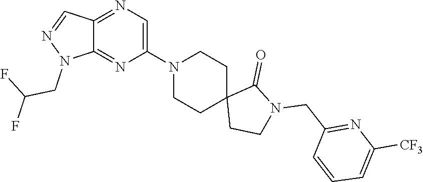 [SMILES: O=C1N(Cc2cccc(C(F)(F)F)n2)CCC12CCN(c1cnc3cnn(CC(F)F)c3n1)CC2] | 354 |
| 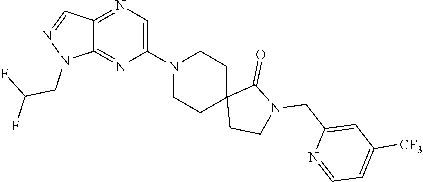 [SMILES: O=C1N(Cc2cc(C(F)(F)F)ccn2)CCC12CCN(c1cnc3cnn(CC(F)F)c3n1)CC2] | 355 |
| 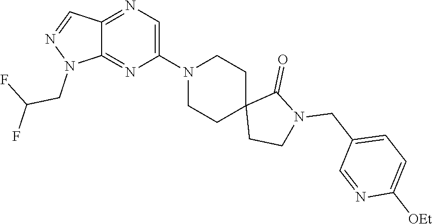 [SMILES: CCOc1ccc(CN2CCC3(CCN(c4cnc5cnn(CC(F)F)c5n4)CC3)C2=O)cn1] | 356 |
| 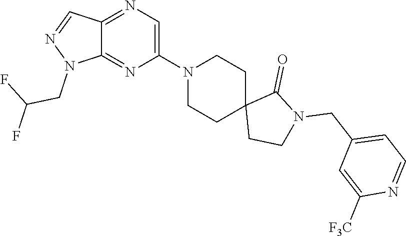 [SMILES: O=C1N(Cc2ccnc(C(F)(F)F)c2)CCC12CCN(c1cnc3cnn(CC(F)F)c3n1)CC2] | 357 |
| 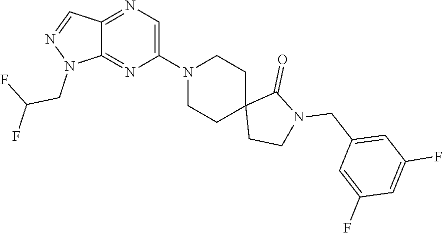 [SMILES: O=C1N(Cc2cc(F)cc(F)c2)CCC12CCN(c1cnc3cnn(CC(F)F)c3n1)CC2] | 358 |
| 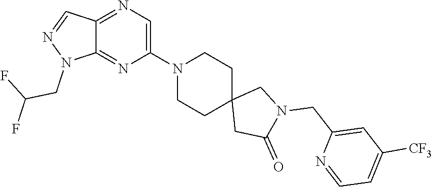 [SMILES: O=C1CC2(CCN(c3cnc4cnn(CC(F)F)c4n3)CC2)CN1Cc1cc(C(F)(F)F)ccn1] | 359 |
| 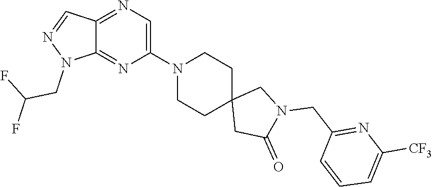 [SMILES: O=C1CC2(CCN(c3cnc4cnn(CC(F)F)c4n3)CC2)CN1Cc1cccc(C(F)(F)F)n1] | 360 |
| 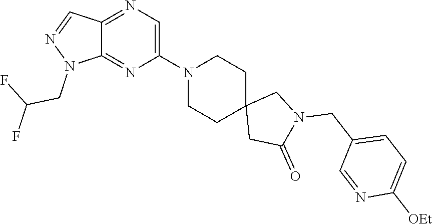 [SMILES: CCOc1ccc(CN2CC3(CCN(c4cnc5cnn(CC(F)F)c5n4)CC3)CC2=O)cn1] | 361 |
| 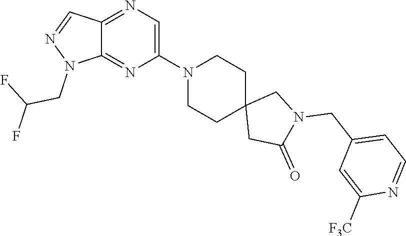 [SMILES: O=C1CC2(CCN(c3cnc4cnn(CC(F)F)c4n3)CC2)CN1Cc1ccnc(C(F)(F)F)c1] | 362 |
| 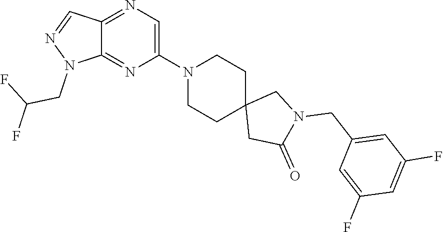 [SMILES: O=C1CC2(CCN(c3cnc4cnn(CC(F)F)c4n3)CC2)CN1Cc1cc(F)cc(F)c1] | 363 |
| 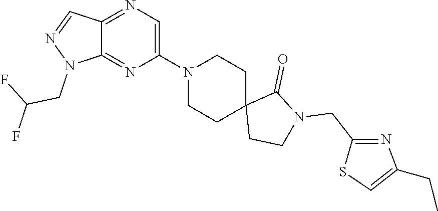 [SMILES: CCc1csc(CN2CCC3(CCN(c4cnc5cnn(CC(F)F)c5n4)CC3)C2=O)n1] | 364 |
| 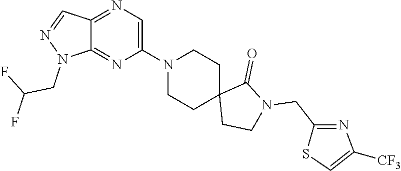 [SMILES: O=C1N(Cc2nc(C(F)(F)F)cs2)CCC12CCN(c1cnc3cnn(CC(F)F)c3n1)CC2] | 365 |
| 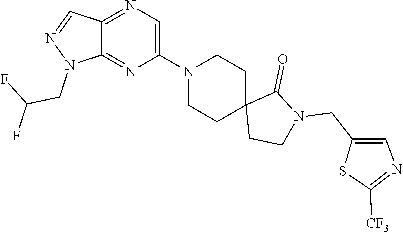 [SMILES: O=C1N(Cc2cnc(C(F)(F)F)s2)CCC12CCN(c1cnc3cnn(CC(F)F)c3n1)CC2] | 366 |
| 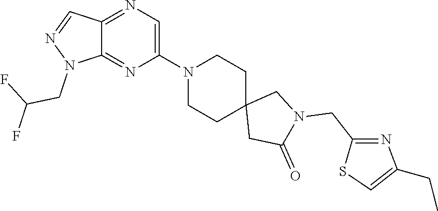 [SMILES: CCc1csc(CN2CC3(CCN(c4cnc5cnn(CC(F)F)c5n4)CC3)CC2=O)n1] | 367 |
| 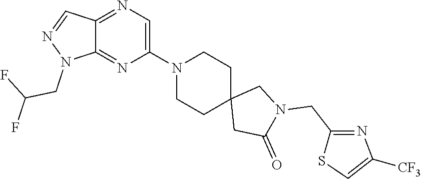 [SMILES: O=C1CC2(CCN(c3cnc4cnn(CC(F)F)c4n3)CC2)CN1Cc1nc(C(F)(F)F)cs1] | 368 |
| 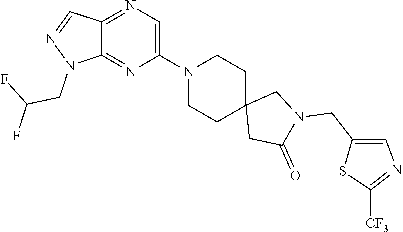 [SMILES: O=C1CC2(CCN(c3cnc4cnn(CC(F)F)c4n3)CC2)CN1Cc1cnc(C(F)(F)F)s1] | 369 |
| 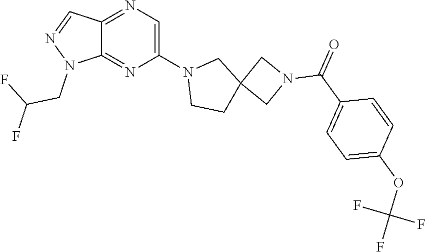 [SMILES: O=C(c1ccc(OC(F)(F)F)cc1)N1CC2(CCN(c3cnc4cnn(CC(F)F)c4n3)C2)C1] | 370 |
| 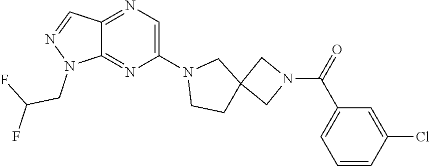 [SMILES: O=C(c1cccc(Cl)c1)N1CC2(CCN(c3cnc4cnn(CC(F)F)c4n3)C2)C1] | 371 |
| 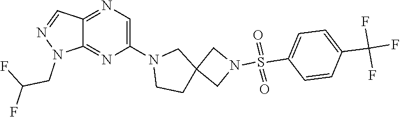 [SMILES: O=S(=O)(c1ccc(C(F)(F)F)cc1)N1CC2(CCN(c3cnc4cnn(CC(F)F)c4n3)C2)C1] | 372 |
| 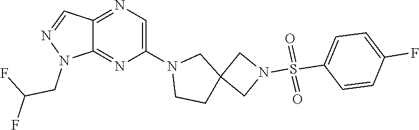 [SMILES: O=S(=O)(c1ccc(F)cc1)N1CC2(CCN(c3cnc4cnn(CC(F)F)c4n3)C2)C1] | 373 |
| 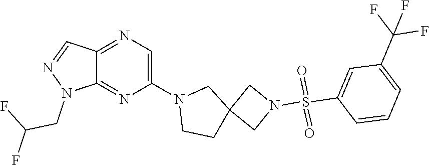 [SMILES: O=S(=O)(c1cccc(C(F)(F)F)c1)N1CC2(CCN(c3cnc4cnn(CC(F)F)c4n3)C2)C1] | 374 |
| 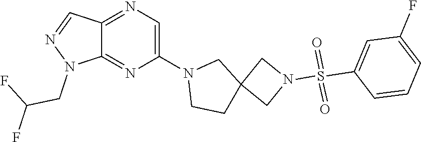 [SMILES: O=S(=O)(c1cccc(F)c1)N1CC2(CCN(c3cnc4cnn(CC(F)F)c4n3)C2)C1] | 375 |
| 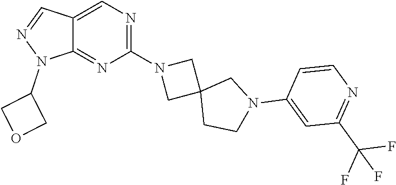 [SMILES: FC(F)(F)c1cc(N2CCC3(C2)CN(c2ncc4cnn(C5COC5)c4n2)C3)ccn1] | 376 |
| 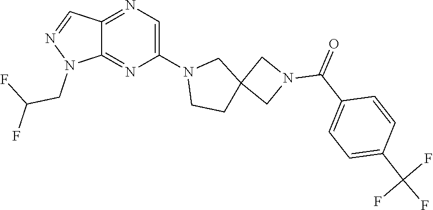 [SMILES: O=C(c1ccc(C(F)(F)F)cc1)N1CC2(CCN(c3cnc4cnn(CC(F)F)c4n3)C2)C1] | 377 |
| 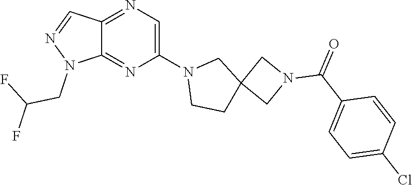 [SMILES: O=C(c1ccc(Cl)cc1)N1CC2(CCN(c3cnc4cnn(CC(F)F)c4n3)C2)C1] | 378 |
| 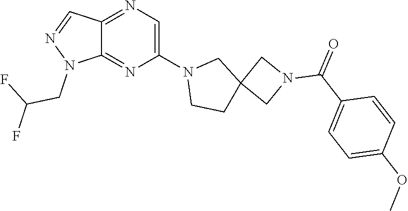 [SMILES: COc1ccc(C(=O)N2CC3(CCN(c4cnc5cnn(CC(F)F)c5n4)C3)C2)cc1] | 379 |
| 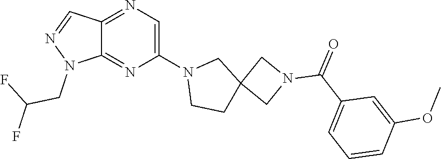 [SMILES: COc1cccc(C(=O)N2CC3(CCN(c4cnc5cnn(CC(F)F)c5n4)C3)C2)c1] | 380 |
| 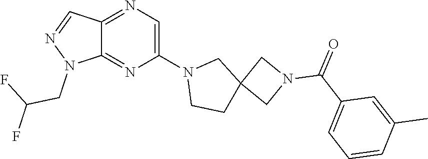 [SMILES: Cc1cccc(C(=O)N2CC3(CCN(c4cnc5cnn(CC(F)F)c5n4)C3)C2)c1] | 381 |
| 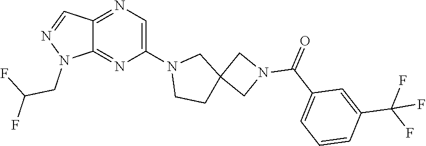 [SMILES: O=C(c1cccc(C(F)(F)F)c1)N1CC2(CCN(c3cnc4cnn(CC(F)F)c4n3)C2)C1] | 382 |
| 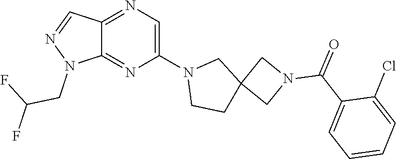 [SMILES: O=C(c1ccccc1Cl)N1CC2(CCN(c3cnc4cnn(CC(F)F)c4n3)C2)C1] | 383 |
| 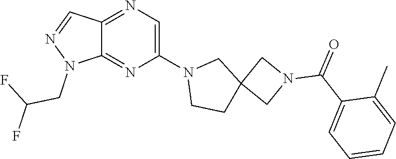 [SMILES: Cc1ccccc1C(=O)N1CC2(CCN(c3cnc4cnn(CC(F)F)c4n3)C2)C1] | 384 |
| 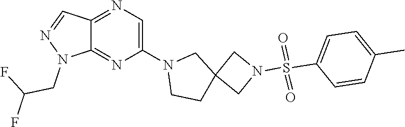 [SMILES: Cc1ccc(S(=O)(=O)N2CC3(CCN(c4cnc5cnn(CC(F)F)c5n4)C3)C2)cc1] | 385 |
| 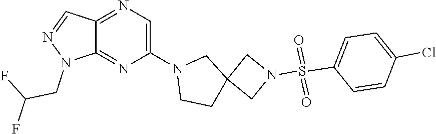 [SMILES: O=S(=O)(c1ccc(Cl)cc1)N1CC2(CCN(c3cnc4cnn(CC(F)F)c4n3)C2)C1] | 386 |
| 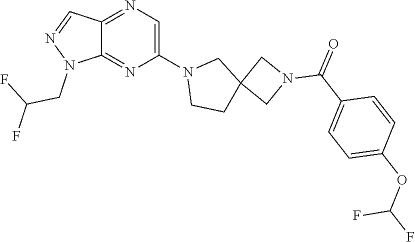 [SMILES: O=C(c1ccc(OC(F)F)cc1)N1CC2(CCN(c3cnc4cnn(CC(F)F)c4n3)C2)C1] | 387 |
| 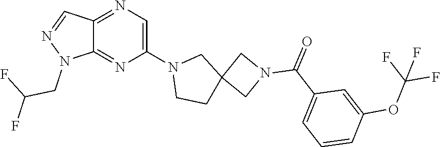 [SMILES: O=C(c1cccc(OC(F)(F)F)c1)N1CC2(CCN(c3cnc4cnn(CC(F)F)c4n3)C2)C1] | 388 |
| 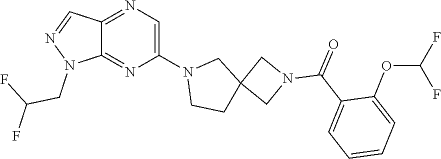 [SMILES: O=C(c1ccccc1OC(F)F)N1CC2(CCN(c3cnc4cnn(CC(F)F)c4n3)C2)C1] | 389 |
| 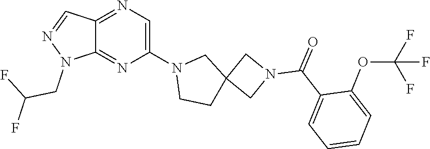 [SMILES: O=C(c1ccccc1OC(F)(F)F)N1CC2(CCN(c3cnc4cnn(CC(F)F)c4n3)C2)C1] | 390 |
| 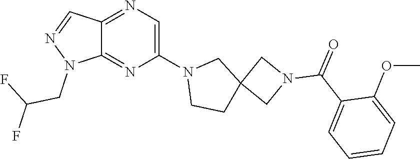 [SMILES: COc1ccccc1C(=O)N1CC2(CCN(c3cnc4cnn(CC(F)F)c4n3)C2)C1] | 391 |
| 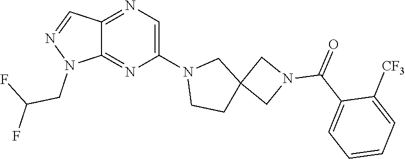 [SMILES: O=C(c1ccccc1C(F)(F)F)N1CC2(CCN(c3cnc4cnn(CC(F)F)c4n3)C2)C1] | 392 |
| 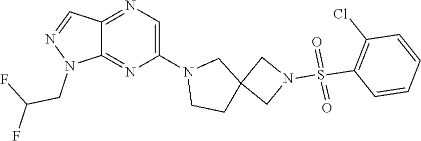 [SMILES: O=S(=O)(c1ccccc1Cl)N1CC2(CCN(c3cnc4cnn(CC(F)F)c4n3)C2)C1] | 393 |
| 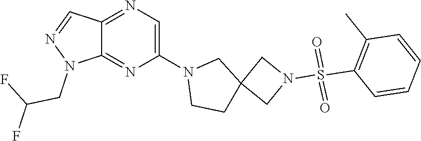 [SMILES: Cc1ccccc1S(=O)(=O)N1CC2(CCN(c3cnc4cnn(CC(F)F)c4n3)C2)C1] | 394 |
| 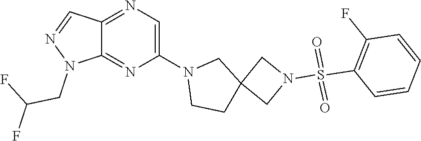 [SMILES: O=S(=O)(c1ccccc1F)N1CC2(CCN(c3cnc4cnn(CC(F)F)c4n3)C2)C1] | 395 |
| 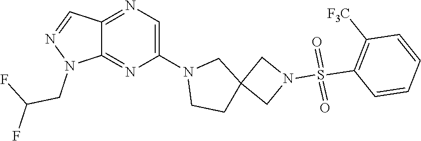 [SMILES: O=S(=O)(c1ccccc1C(F)(F)F)N1CC2(CCN(c3cnc4cnn(CC(F)F)c4n3)C2)C1] | 396 |
| 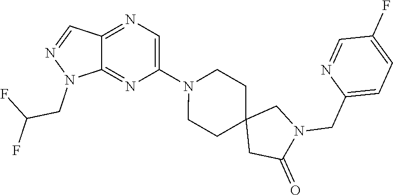 [SMILES: O=C1CC2(CCN(c3cnc4cnn(CC(F)F)c4n3)CC2)CN1Cc1ccc(F)cn1] | 397 |
| 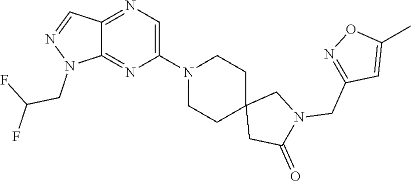 [SMILES: Cc1cc(CN2CC3(CCN(c4cnc5cnn(CC(F)F)c5n4)CC3)CC2=O)no1] | 398 |
| 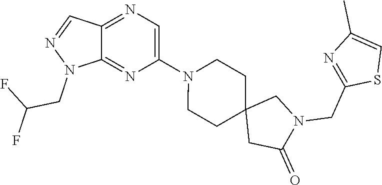 [SMILES: Cc1csc(CN2CC3(CCN(c4cnc5cnn(CC(F)F)c5n4)CC3)CC2=O)n1] | 399 |
| 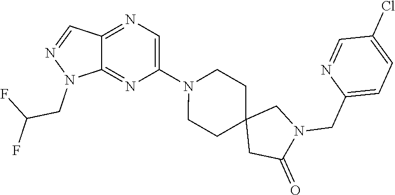 [SMILES: O=C1CC2(CCN(c3cnc4cnn(CC(F)F)c4n3)CC2)CN1Cc1ccc(Cl)cn1] | 400 |
| 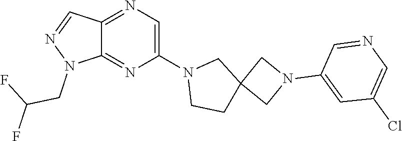 [SMILES: FC(F)Cn1ncc2ncc(N3CCC4(CN(c5cncc(Cl)c5)C4)C3)nc21] | 401 |
| 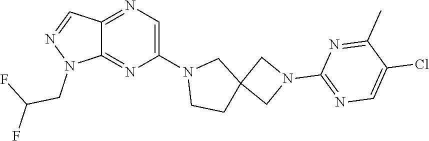 [SMILES: Cc1nc(N2CC3(CCN(c4cnc5cnn(CC(F)F)c5n4)C3)C2)ncc1Cl] | 402 |
| 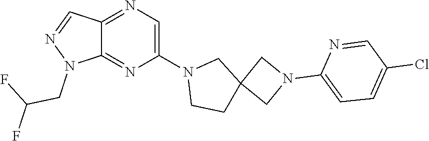 [SMILES: FC(F)Cn1ncc2ncc(N3CCC4(CN(c5ccc(Cl)cn5)C4)C3)nc21] | 403 |
| 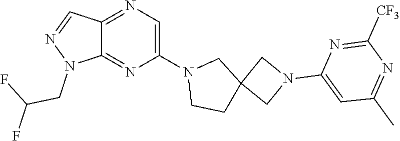 [SMILES: Cc1cc(N2CC3(CCN(c4cnc5cnn(CC(F)F)c5n4)C3)C2)nc(C(F)(F)F)n1] | 404 |
| 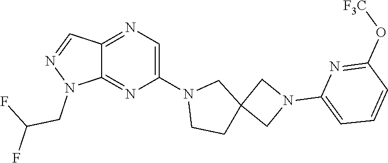 [SMILES: FC(F)Cn1ncc2ncc(N3CCC4(CN(c5cccc(OC(F)(F)F)n5)C4)C3)nc21] | 405 |
| 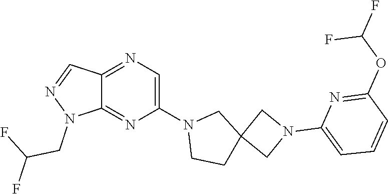 [SMILES: FC(F)Cn1ncc2ncc(N3CCC4(CN(c5cccc(OC(F)F)n5)C4)C3)nc21] | 406 |
| 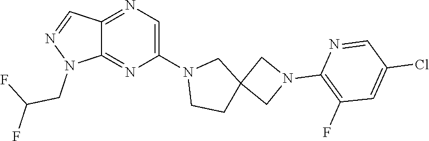 [SMILES: Fc1cc(Cl)cnc1N1CC2(CCN(c3cnc4cnn(CC(F)F)c4n3)C2)C1] | 407 |
| 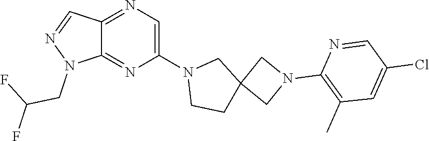 [SMILES: Cc1cc(Cl)cnc1N1CC2(CCN(c3cnc4cnn(CC(F)F)c4n3)C2)C1] | 408 |
| 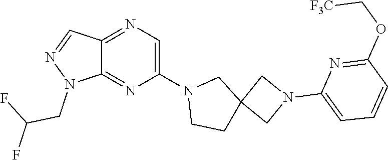 [SMILES: FC(F)Cn1ncc2ncc(N3CCC4(CN(c5cccc(OCC(F)(F)F)n5)C4)C3)nc21] | 409 |
| 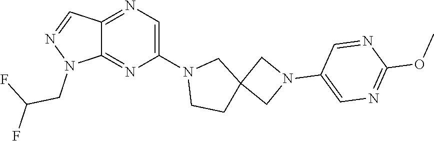 [SMILES: COc1ncc(N2CC3(CCN(c4cnc5cnn(CC(F)F)c5n4)C3)C2)cn1] | 410 |
| 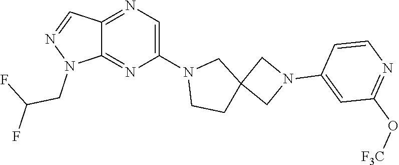 [SMILES: FC(F)Cn1ncc2ncc(N3CCC4(CN(c5ccnc(OC(F)(F)F)c5)C4)C3)nc21] | 411 |
| 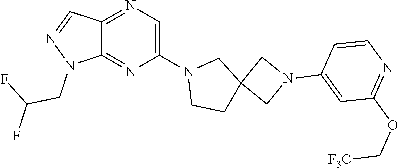 [SMILES: FC(F)Cn1ncc2ncc(N3CCC4(CN(c5ccnc(OCC(F)(F)F)c5)C4)C3)nc21] | 412 |
| 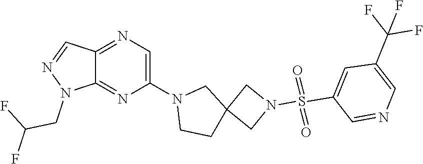 [SMILES: O=S(=O)(c1cncc(C(F)(F)F)c1)N1CC2(CCN(c3cnc4cnn(CC(F)F)c4n3)C2)C1] | 413 |
| 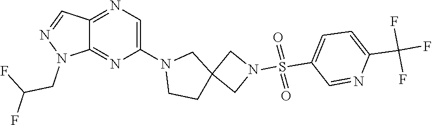 [SMILES: O=S(=O)(c1ccc(C(F)(F)F)nc1)N1CC2(CCN(c3cnc4cnn(CC(F)F)c4n3)C2)C1] | 414 |
| 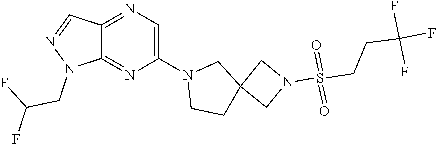 [SMILES: O=S(=O)(CCC(F)(F)F)N1CC2(CCN(c3cnc4cnn(CC(F)F)c4n3)C2)C1] | 415 |
| 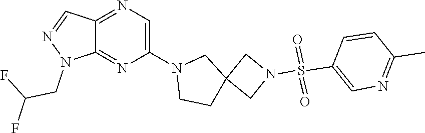 [SMILES: Cc1ccc(S(=O)(=O)N2CC3(CCN(c4cnc5cnn(CC(F)F)c5n4)C3)C2)cn1] | 416 |
| 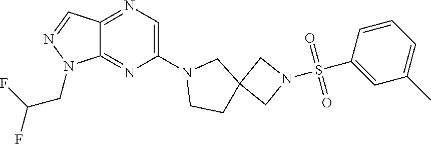 [SMILES: Cc1cccc(S(=O)(=O)N2CC3(CCN(c4cnc5cnn(CC(F)F)c5n4)C3)C2)c1] | 417 |
| 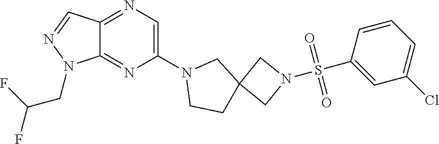 [SMILES: O=S(=O)(c1cccc(Cl)c1)N1CC2(CCN(c3cnc4cnn(CC(F)F)c4n3)C2)C1] | 418 |
| 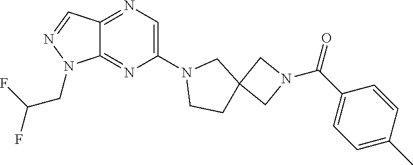 [SMILES: Cc1ccc(C(=O)N2CC3(CCN(c4cnc5cnn(CC(F)F)c5n4)C3)C2)cc1] | 419 |
| 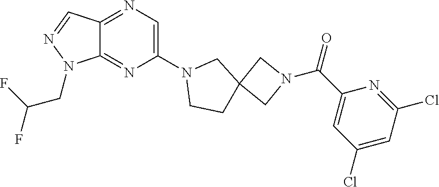 [SMILES: O=C(c1cc(Cl)cc(Cl)n1)N1CC2(CCN(c3cnc4cnn(CC(F)F)c4n3)C2)C1] | 420 |
| 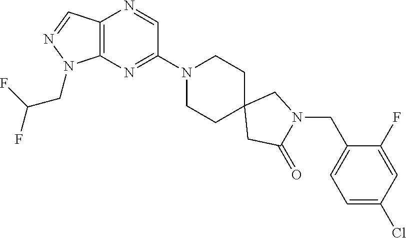 [SMILES: O=C1CC2(CCN(c3cnc4cnn(CC(F)F)c4n3)CC2)CN1Cc1ccc(Cl)cc1F] | 421 |
| 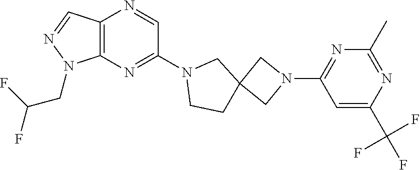 [SMILES: Cc1nc(N2CC3(CCN(c4cnc5cnn(CC(F)F)c5n4)C3)C2)cc(C(F)(F)F)n1] | 422 |
| 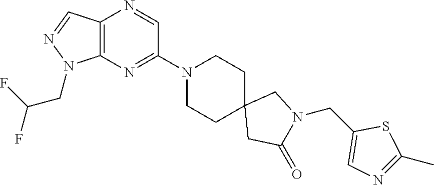 [SMILES: Cc1ncc(CN2CC3(CCN(c4cnc5cnn(CC(F)F)c5n4)CC3)CC2=O)s1] | 423 |
| 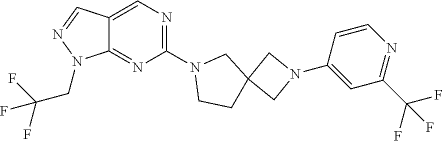 [SMILES: FC(F)(F)Cn1ncc2cnc(N3CCC4(CN(c5ccnc(C(F)(F)F)c5)C4)C3)nc21] | 424 |
| 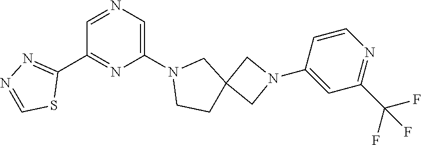 [SMILES: FC(F)(F)c1cc(N2CC3(CCN(c4cncc(-c5nncs5)n4)C3)C2)ccn1] | 425 |
| 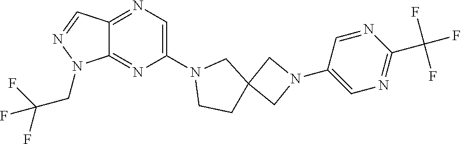 [SMILES: FC(F)(F)Cn1ncc2ncc(N3CCC4(CN(c5cnc(C(F)(F)F)nc5)C4)C3)nc21] | 426 |
| 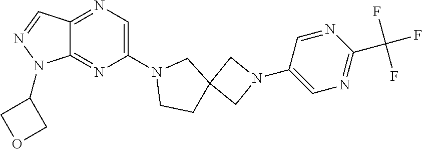 [SMILES: FC(F)(F)c1ncc(N2CC3(CCN(c4cnc5cnn(C6COC6)c5n4)C3)C2)cn1] | 427 |
| 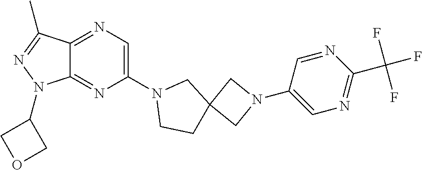 [SMILES: Cc1nn(C2COC2)c2nc(N3CCC4(CN(c5cnc(C(F)(F)F)nc5)C4)C3)cnc12] | 428 |
| 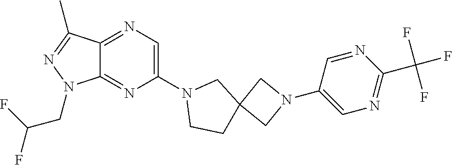 [SMILES: Cc1nn(CC(F)F)c2nc(N3CCC4(CN(c5cnc(C(F)(F)F)nc5)C4)C3)cnc12] | 429 |
| 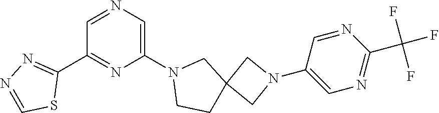 [SMILES: FC(F)(F)c1ncc(N2CC3(CCN(c4cncc(-c5nncs5)n4)C3)C2)cn1] | 430 |
| 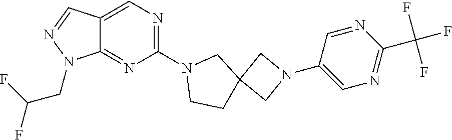 [SMILES: FC(F)Cn1ncc2cnc(N3CCC4(CN(c5cnc(C(F)(F)F)nc5)C4)C3)nc21] | 431 |
| 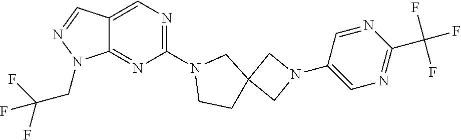 [SMILES: FC(F)(F)Cn1ncc2cnc(N3CCC4(CN(c5cnc(C(F)(F)F)nc5)C4)C3)nc21] | 432 |
| 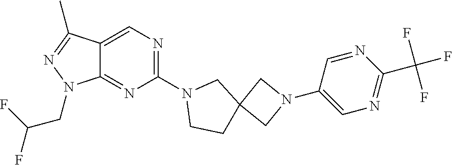 [SMILES: Cc1nn(CC(F)F)c2nc(N3CCC4(CN(c5cnc(C(F)(F)F)nc5)C4)C3)ncc12] | 433 |
| 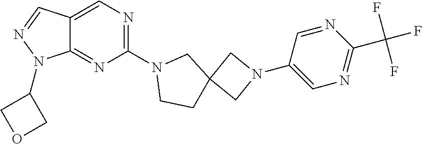 [SMILES: FC(F)(F)c1ncc(N2CC3(CCN(c4ncc5cnn(C6COC6)c5n4)C3)C2)cn1] | 434 |
| 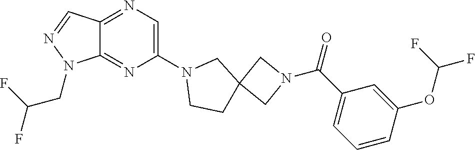 [SMILES: O=C(c1cccc(OC(F)F)c1)N1CC2(CCN(c3cnc4cnn(CC(F)F)c4n3)C2)C1] | 435 |
| 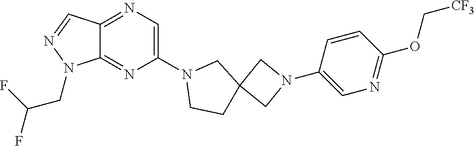 [SMILES: FC(F)Cn1ncc2ncc(N3CCC4(CN(c5ccc(OCC(F)(F)F)nc5)C4)C3)nc21] | 436 |
| 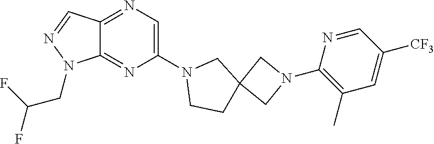 [SMILES: Cc1cc(C(F)(F)F)cnc1N1CC2(CCN(c3cnc4cnn(CC(F)F)c4n3)C2)C1] | 437 |
| 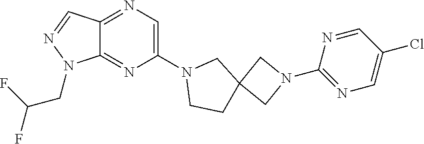 [SMILES: FC(F)Cn1ncc2ncc(N3CCC4(C3)CN(c3ncc(Cl)cn3)C4)nc21] | 438 |
| 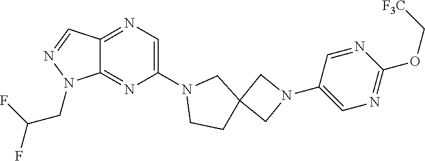 [SMILES: FC(F)Cn1ncc2ncc(N3CCC4(CN(c5cnc(OCC(F)(F)F)nc5)C4)C3)nc21] | 439 |
| 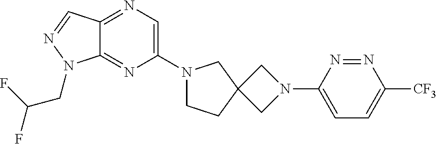 [SMILES: FC(F)Cn1ncc2ncc(N3CCC4(CN(c5ccc(C(F)(F)F)nn5)C4)C3)nc21] | 440 |
| 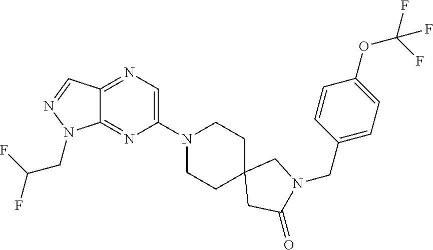 [SMILES: O=C1CC2(CCN(c3cnc4cnn(CC(F)F)c4n3)CC2)CN1Cc1ccc(OC(F)(F)F)cc1] | 441 |
| 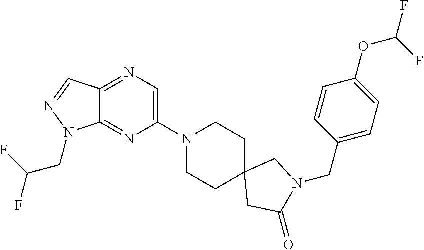 [SMILES: O=C1CC2(CCN(c3cnc4cnn(CC(F)F)c4n3)CC2)CN1Cc1ccc(OC(F)F)cc1] | 442 |
| 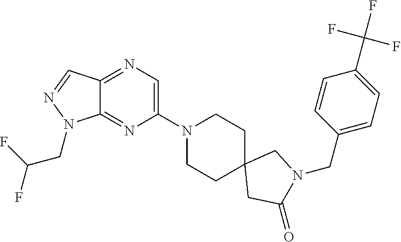 [SMILES: O=C1CC2(CCN(c3cnc4cnn(CC(F)F)c4n3)CC2)CN1Cc1ccc(C(F)(F)F)cc1] | 443 |
| 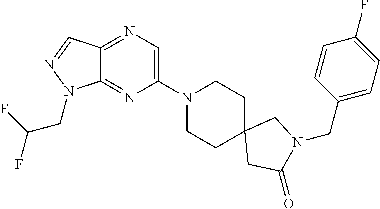 [SMILES: O=C1CC2(CCN(c3cnc4cnn(CC(F)F)c4n3)CC2)CN1Cc1ccc(F)cc1] | 444 |
| 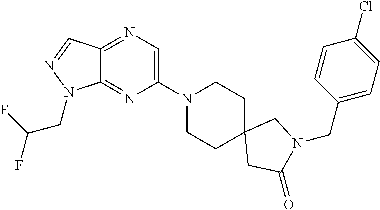 [SMILES: O=C1CC2(CCN(c3cnc4cnn(CC(F)F)c4n3)CC2)CN1Cc1ccc(Cl)cc1] | 445 |
| 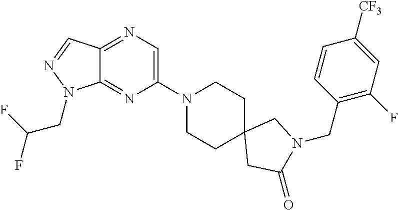 [SMILES: O=C1CC2(CCN(c3cnc4cnn(CC(F)F)c4n3)CC2)CN1Cc1ccc(C(F)(F)F)cc1F] | 446 |
| 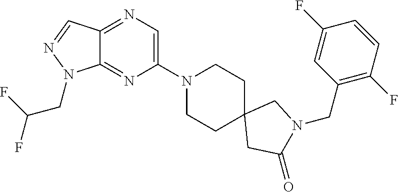 [SMILES: O=C1CC2(CCN(c3cnc4cnn(CC(F)F)c4n3)CC2)CN1Cc1cc(F)ccc1F] | 447 |
| 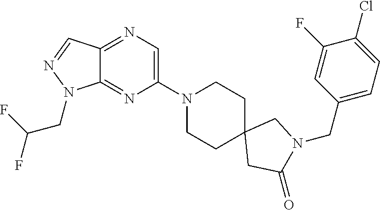 [SMILES: O=C1CC2(CCN(c3cnc4cnn(CC(F)F)c4n3)CC2)CN1Cc1ccc(Cl)c(F)c1] | 448 |
| 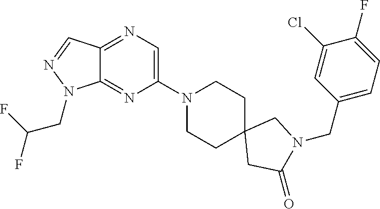 [SMILES: O=C1CC2(CCN(c3cnc4cnn(CC(F)F)c4n3)CC2)CN1Cc1ccc(F)c(Cl)c1] | 449 |
| 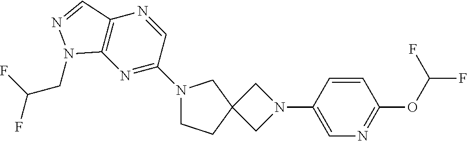 [SMILES: FC(F)Cn1ncc2ncc(N3CCC4(CN(c5ccc(OC(F)F)nc5)C4)C3)nc21] | 450 |
| 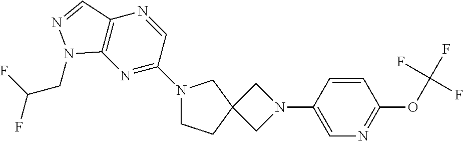 [SMILES: FC(F)Cn1ncc2ncc(N3CCC4(CN(c5ccc(OC(F)(F)F)nc5)C4)C3)nc21] | 451 |
| 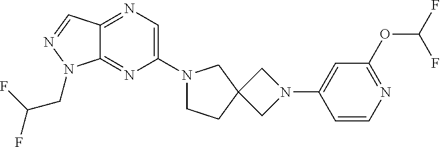 [SMILES: FC(F)Cn1ncc2ncc(N3CCC4(CN(c5ccnc(OC(F)F)c5)C4)C3)nc21] | 452 |
| 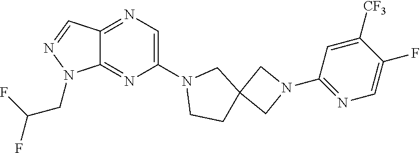 [SMILES: Fc1cnc(N2CC3(CCN(c4cnc5cnn(CC(F)F)c5n4)C3)C2)cc1C(F)(F)F] | 453 |
| 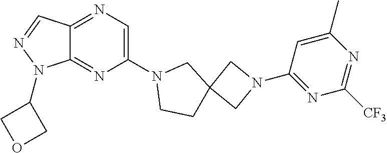 [SMILES: Cc1cc(N2CC3(CCN(c4cnc5cnn(C6COC6)c5n4)C3)C2)nc(C(F)(F)F)n1] | 454 |
| 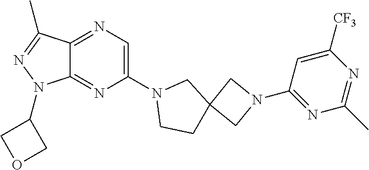 [SMILES: Cc1nc(N2CC3(CCN(c4cnc5c(C)nn(C6COC6)c5n4)C3)C2)cc(C(F)(F)F)n1] | 455 |
| 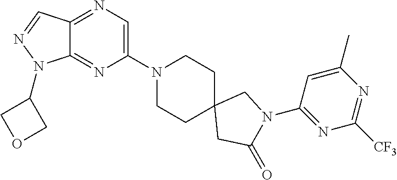 [SMILES: Cc1cc(N2CC3(CCN(c4cnc5cnn(C6COC6)c5n4)CC3)CC2=O)nc(C(F)(F)F)n1] | 456 |
| 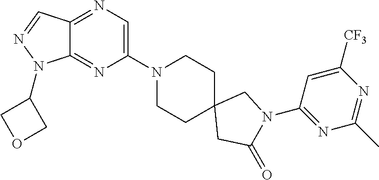 [SMILES: Cc1nc(N2CC3(CCN(c4cnc5cnn(C6COC6)c5n4)CC3)CC2=O)cc(C(F)(F)F)n1] | 457 |
| 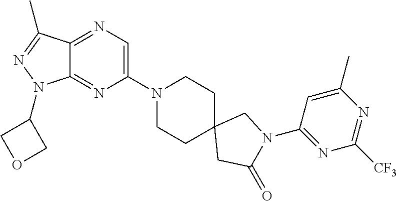 [SMILES: Cc1cc(N2CC3(CCN(c4cnc5c(C)nn(C6COC6)c5n4)CC3)CC2=O)nc(C(F)(F)F)n1] | 458 |
| 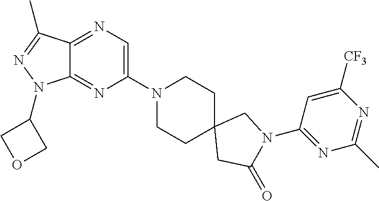 [SMILES: Cc1nc(N2CC3(CCN(c4cnc5c(C)nn(C6COC6)c5n4)CC3)CC2=O)cc(C(F)(F)F)n1] | 459 |
| 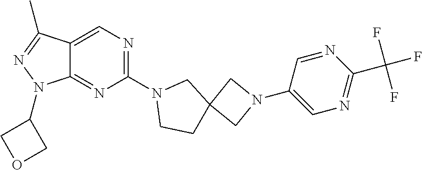 [SMILES: Cc1nn(C2COC2)c2nc(N3CCC4(CN(c5cnc(C(F)(F)F)nc5)C4)C3)ncc12] | 460 |
| 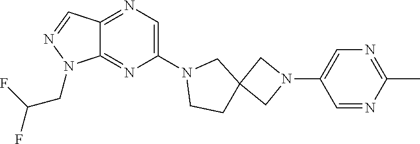 [SMILES: Cc1ncc(N2CC3(CCN(c4cnc5cnn(CC(F)F)c5n4)C3)C2)cn1] | 461 |
| 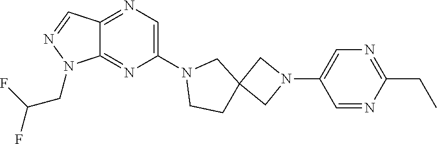 [SMILES: CCc1ncc(N2CC3(CCN(c4cnc5cnn(CC(F)F)c5n4)C3)C2)cn1] | 462 |
| 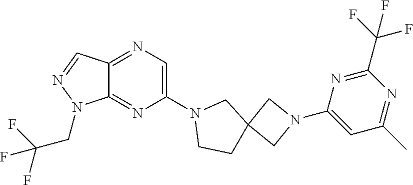 [SMILES: Cc1cc(N2CC3(CCN(c4cnc5cnn(CC(F)(F)F)c5n4)C3)C2)nc(C(F)(F)F)n1] | 463 |
| 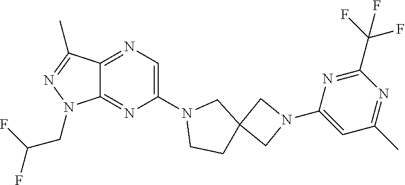 [SMILES: Cc1cc(N2CC3(CCN(c4cnc5c(C)nn(CC(F)F)c5n4)C3)C2)nc(C(F)(F)F)n1] | 464 |
| 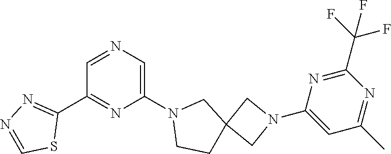 [SMILES: Cc1cc(N2CC3(CCN(c4cncc(-c5nncs5)n4)C3)C2)nc(C(F)(F)F)n1] | 465 |
| 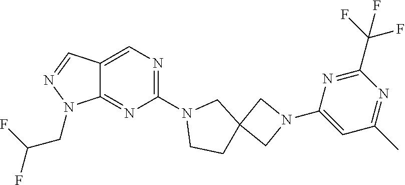 [SMILES: Cc1cc(N2CC3(CCN(c4ncc5cnn(CC(F)F)c5n4)C3)C2)nc(C(F)(F)F)n1] | 466 |
| 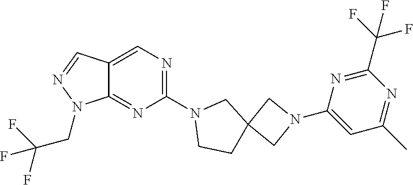 [SMILES: Cc1cc(N2CC3(CCN(c4ncc5cnn(CC(F)(F)F)c5n4)C3)C2)nc(C(F)(F)F)n1] | 467 |
| 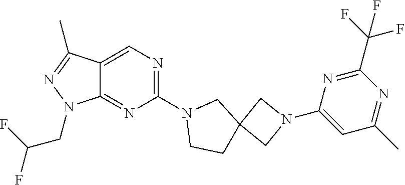 [SMILES: Cc1cc(N2CC3(CCN(c4ncc5c(C)nn(CC(F)F)c5n4)C3)C2)nc(C(F)(F)F)n1] | 468 |
| 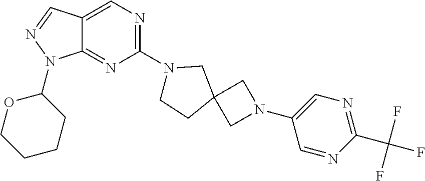 [SMILES: FC(F)(F)c1ncc(N2CC3(CCN(c4ncc5cnn(C6CCCCO6)c5n4)C3)C2)cn1] | 469 |
| 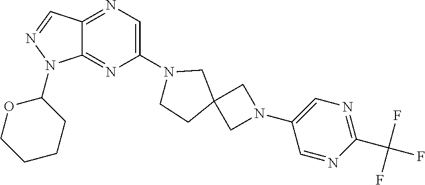 [SMILES: FC(F)(F)c1ncc(N2CC3(CCN(c4cnc5cnn(C6CCCCO6)c5n4)C3)C2)cn1] | 470 |
| 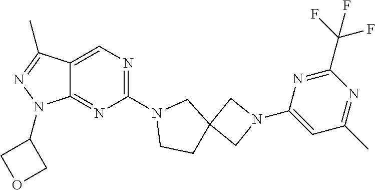 [SMILES: Cc1cc(N2CC3(CCN(c4ncc5c(C)nn(C6COC6)c5n4)C3)C2)nc(C(F)(F)F)n1] | 471 |
| 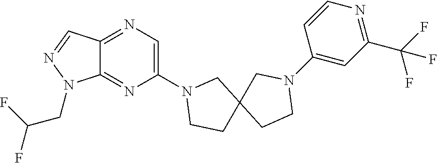 [SMILES: FC(F)Cn1ncc2ncc(N3CCC4(CCN(c5ccnc(C(F)(F)F)c5)C4)C3)nc21] | 472 |
| 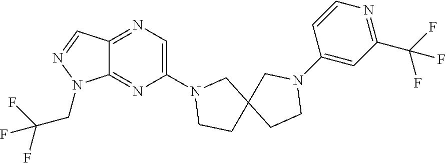 [SMILES: FC(F)(F)Cn1ncc2ncc(N3CCC4(CCN(c5ccnc(C(F)(F)F)c5)C4)C3)nc21] | 473 |
| 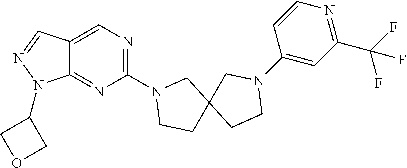 [SMILES: FC(F)(F)c1cc(N2CCC3(CCN(c4ncc5cnn(C6COC6)c5n4)C3)C2)ccn1] | 474 |
| 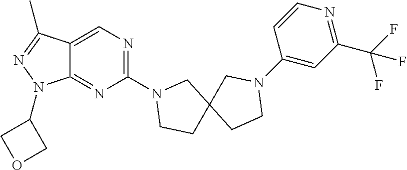 [SMILES: Cc1nn(C2COC2)c2nc(N3CCC4(CCN(c5ccnc(C(F)(F)F)c5)C4)C3)ncc12] | 475 |
| 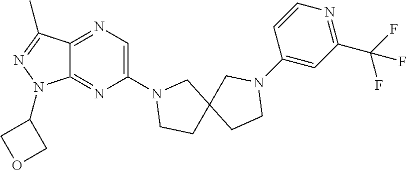 [SMILES: Cc1nn(C2COC2)c2nc(N3CCC4(CCN(c5ccnc(C(F)(F)F)c5)C4)C3)cnc12] | 476 |
| 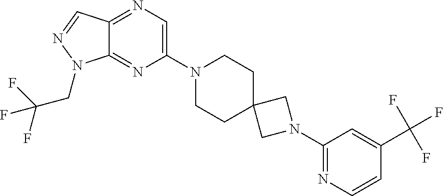 [SMILES: FC(F)(F)Cn1ncc2ncc(N3CCC4(CC3)CN(c3cc(C(F)(F)F)ccn3)C4)nc21] | 477 |
| 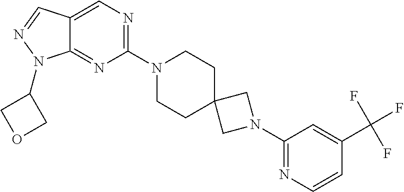 [SMILES: FC(F)(F)c1ccnc(N2CC3(CCN(c4ncc5cnn(C6COC6)c5n4)CC3)C2)c1] | 478 |
| 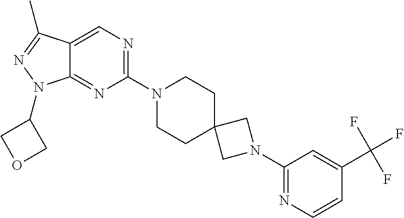 [SMILES: Cc1nn(C2COC2)c2nc(N3CCC4(CC3)CN(c3cc(C(F)(F)F)ccn3)C4)ncc12] | 479 |
| 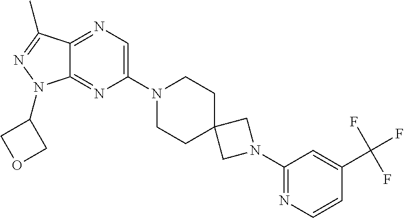 [SMILES: Cc1nn(C2COC2)c2nc(N3CCC4(CC3)CN(c3cc(C(F)(F)F)ccn3)C4)cnc12] | 480 |
| 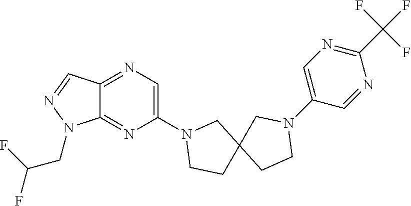 [SMILES: FC(F)Cn1ncc2ncc(N3CCC4(CCN(c5cnc(C(F)(F)F)nc5)C4)C3)nc21] | 481 |
| 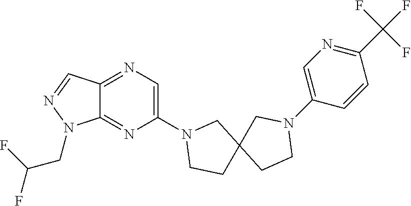 [SMILES: FC(F)Cn1ncc2ncc(N3CCC4(CCN(c5ccc(C(F)(F)F)nc5)C4)C3)nc21] | 482 |
| 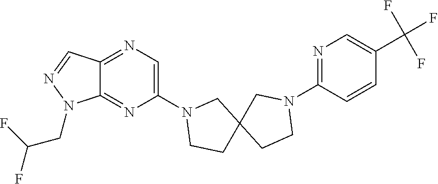 [SMILES: FC(F)Cn1ncc2ncc(N3CCC4(CCN(c5ccc(C(F)(F)F)cn5)C4)C3)nc21] | 483 |
| 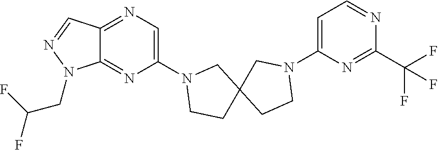 [SMILES: FC(F)Cn1ncc2ncc(N3CCC4(CCN(c5ccnc(C(F)(F)F)n5)C4)C3)nc21] | 484 |
| 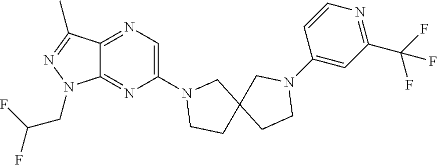 [SMILES: Cc1nn(CC(F)F)c2nc(N3CCC4(CCN(c5ccnc(C(F)(F)F)c5)C4)C3)cnc12] | 485 |
| 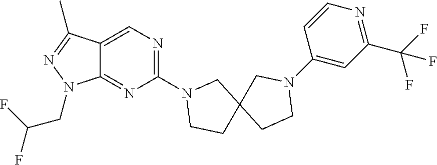 [SMILES: Cc1nn(CC(F)F)c2nc(N3CCC4(CCN(c5ccnc(C(F)(F)F)c5)C4)C3)ncc12] | 486 |
| 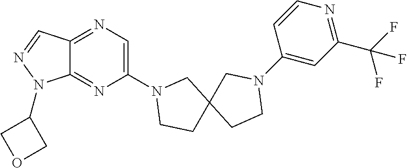 [SMILES: FC(F)(F)c1cc(N2CCC3(CCN(c4cnc5cnn(C6COC6)c5n4)C3)C2)ccn1] | 487 |
| 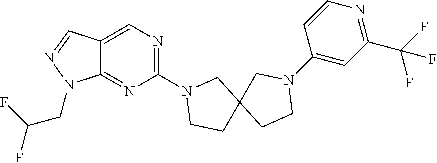 [SMILES: FC(F)Cn1ncc2cnc(N3CCC4(CCN(c5ccnc(C(F)(F)F)c5)C4)C3)nc21] | 488 |
| 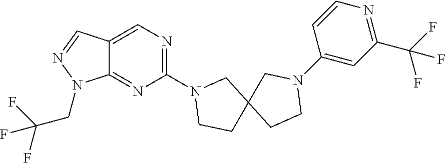 [SMILES: FC(F)(F)Cn1ncc2cnc(N3CCC4(CCN(c5ccnc(C(F)(F)F)c5)C4)C3)nc21] | 489 |
| 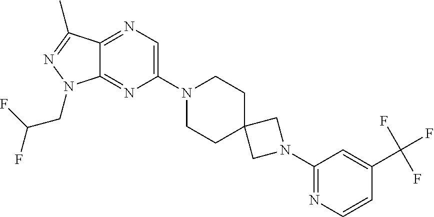 [SMILES: Cc1nn(CC(F)F)c2nc(N3CCC4(CC3)CN(c3cc(C(F)(F)F)ccn3)C4)cnc12] | 490 |
| 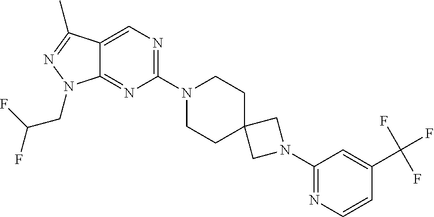 [SMILES: Cc1nn(CC(F)F)c2nc(N3CCC4(CC3)CN(c3cc(C(F)(F)F)ccn3)C4)ncc12] | 491 |
| 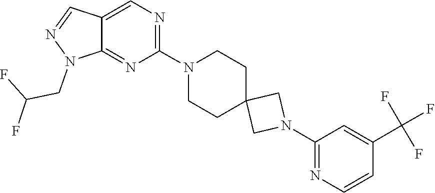 [SMILES: FC(F)Cn1ncc2cnc(N3CCC4(CC3)CN(c3cc(C(F)(F)F)ccn3)C4)nc21] | 492 |
| 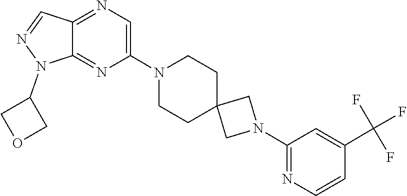 [SMILES: FC(F)(F)c1ccnc(N2CC3(CCN(c4cnc5cnn(C6COC6)c5n4)CC3)C2)c1] | 493 |
| 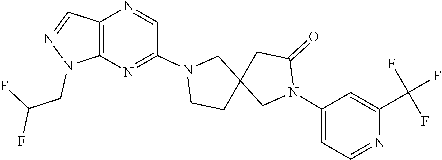 [SMILES: O=C1CC2(CCN(c3cnc4cnn(CC(F)F)c4n3)C2)CN1c1ccnc(C(F)(F)F)c1] | 494 |
| 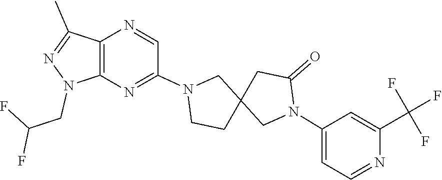 [SMILES: Cc1nn(CC(F)F)c2nc(N3CCC4(CC(=O)N(c5ccnc(C(F)(F)F)c5)C4)C3)cnc12] | 495 |
| 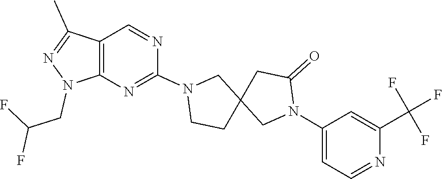 [SMILES: Cc1nn(CC(F)F)c2nc(N3CCC4(CC(=O)N(c5ccnc(C(F)(F)F)c5)C4)C3)ncc12] | 496 |
| 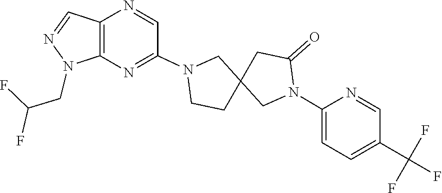 [SMILES: O=C1CC2(CCN(c3cnc4cnn(CC(F)F)c4n3)C2)CN1c1ccc(C(F)(F)F)cn1] | 497 |
| 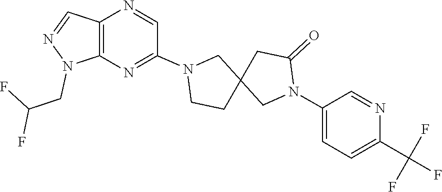 [SMILES: O=C1CC2(CCN(c3cnc4cnn(CC(F)F)c4n3)C2)CN1c1ccc(C(F)(F)F)nc1] | 498 |
| 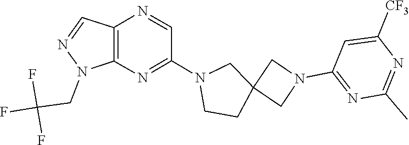 [SMILES: Cc1nc(N2CC3(CCN(c4cnc5cnn(CC(F)(F)F)c5n4)C3)C2)cc(C(F)(F)F)n1] | 499 |
| 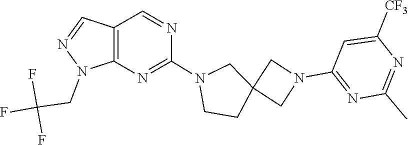 [SMILES: Cc1nc(N2CC3(CCN(c4ncc5cnn(CC(F)(F)F)c5n4)C3)C2)cc(C(F)(F)F)n1] | 500 |
| 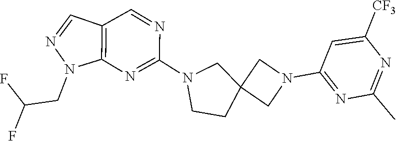 [SMILES: Cc1nc(N2CC3(CCN(c4ncc5cnn(CC(F)F)c5n4)C3)C2)cc(C(F)(F)F)n1] | 501 |
| 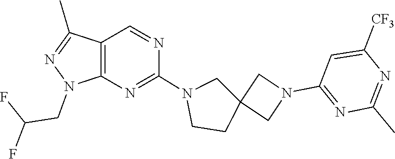 [SMILES: Cc1nc(N2CC3(CCN(c4ncc5c(C)nn(CC(F)F)c5n4)C3)C2)cc(C(F)(F)F)n1] | 502 |
| 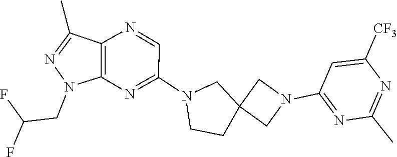 [SMILES: Cc1nc(N2CC3(CCN(c4cnc5c(C)nn(CC(F)F)c5n4)C3)C2)cc(C(F)(F)F)n1] | 503 |
| 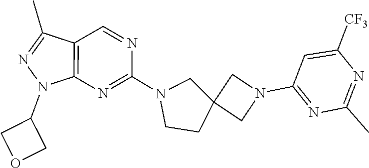 [SMILES: Cc1nc(N2CC3(CCN(c4ncc5c(C)nn(C6COC6)c5n4)C3)C2)cc(C(F)(F)F)n1] | 504 |
| 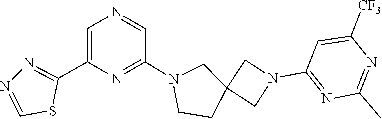 [SMILES: Cc1nc(N2CC3(CCN(c4cncc(-c5nncs5)n4)C3)C2)cc(C(F)(F)F)n1] | 505 |
| 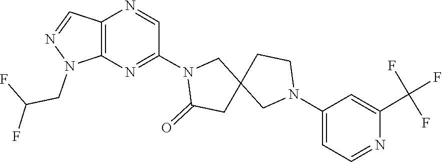 [SMILES: O=C1CC2(CCN(c3ccnc(C(F)(F)F)c3)C2)CN1c1cnc2cnn(CC(F)F)c2n1] | 506 |
| 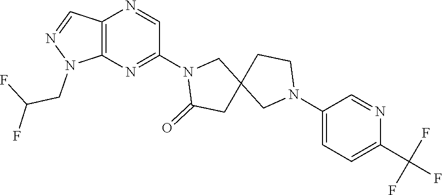 [SMILES: O=C1CC2(CCN(c3ccc(C(F)(F)F)nc3)C2)CN1c1cnc2cnn(CC(F)F)c2n1] | 507 |
| 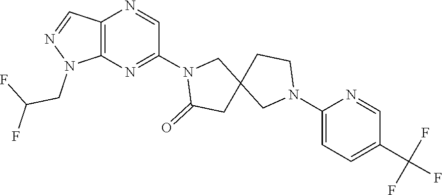 [SMILES: O=C1CC2(CCN(c3ccc(C(F)(F)F)cn3)C2)CN1c1cnc2cnn(CC(F)F)c2n1] | 508 |
| 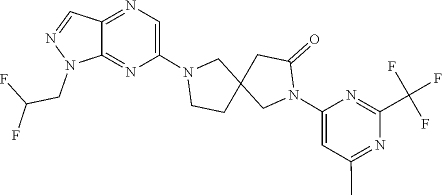 [SMILES: Cc1cc(N2CC3(CCN(c4cnc5cnn(CC(F)F)c5n4)C3)CC2=O)nc(C(F)(F)F)n1] | 509 |
| 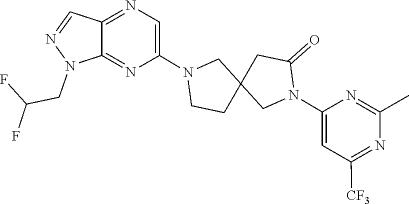 [SMILES: Cc1nc(N2CC3(CCN(c4cnc5cnn(CC(F)F)c5n4)C3)CC2=O)cc(C(F)(F)F)n1] | 510 |
| 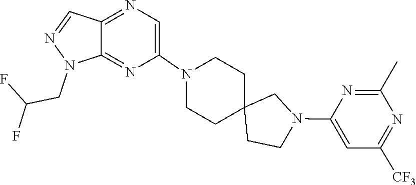 [SMILES: Cc1nc(N2CCC3(CCN(c4cnc5cnn(CC(F)F)c5n4)CC3)C2)cc(C(F)(F)F)n1] | 511 |
| 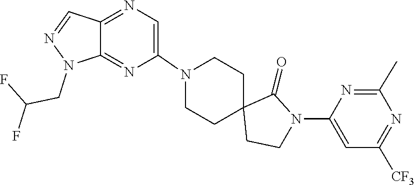 [SMILES: Cc1nc(N2CCC3(CCN(c4cnc5cnn(CC(F)F)c5n4)CC3)C2=O)cc(C(F)(F)F)n1] | 512 |
| 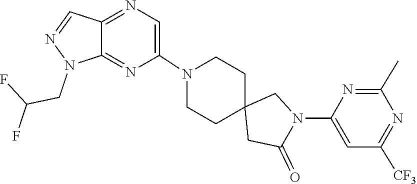 [SMILES: Cc1nc(N2CC3(CCN(c4cnc5cnn(CC(F)F)c5n4)CC3)CC2=O)cc(C(F)(F)F)n1] | 513 |
| 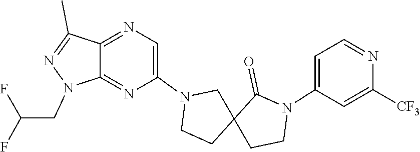 [SMILES: Cc1nn(CC(F)F)c2nc(N3CCC4(CCN(c5ccnc(C(F)(F)F)c5)C4=O)C3)cnc12] | 514 |
| 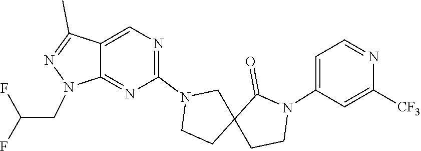 [SMILES: Cc1nn(CC(F)F)c2nc(N3CCC4(CCN(c5ccnc(C(F)(F)F)c5)C4=O)C3)ncc12] | 515 |
| 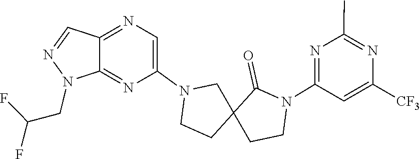 [SMILES: Cc1nc(N2CCC3(CCN(c4cnc5cnn(CC(F)F)c5n4)C3)C2=O)cc(C(F)(F)F)n1] | 516 |
| 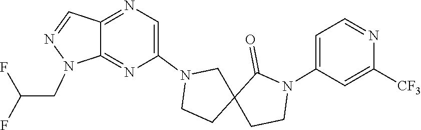 [SMILES: O=C1N(c2ccnc(C(F)(F)F)c2)CCC12CCN(c1cnc3cnn(CC(F)F)c3n1)C2] | 517 |
| 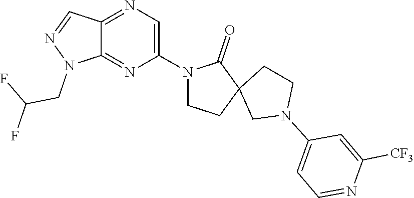 [SMILES: O=C1N(c2cnc3cnn(CC(F)F)c3n2)CCC12CCN(c1ccnc(C(F)(F)F)c1)C2] | 518 |
| 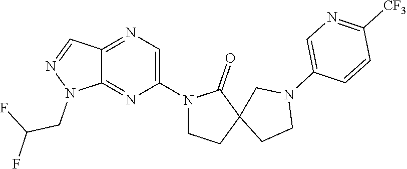 [SMILES: O=C1N(c2cnc3cnn(CC(F)F)c3n2)CCC12CCN(c1ccc(C(F)(F)F)nc1)C2] | 519 |
| 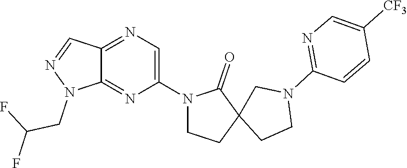 [SMILES: O=C1N(c2cnc3cnn(CC(F)F)c3n2)CCC12CCN(c1ccc(C(F)(F)F)cn1)C2] | 520 |
| 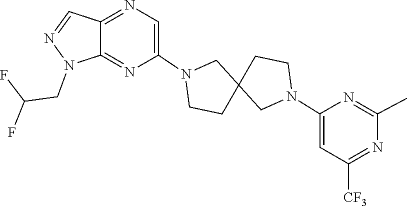 [SMILES: Cc1nc(N2CCC3(CCN(c4cnc5cnn(CC(F)F)c5n4)C3)C2)cc(C(F)(F)F)n1] | 521 |
| 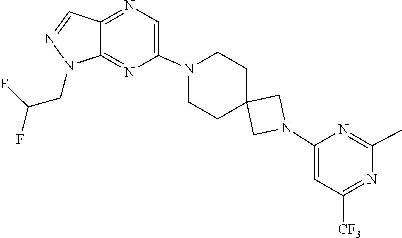 [SMILES: Cc1nc(N2CC3(CCN(c4cnc5cnn(CC(F)F)c5n4)CC3)C2)cc(C(F)(F)F)n1] | 522 |
| 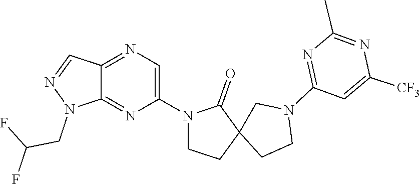 [SMILES: Cc1nc(N2CCC3(CCN(c4cnc5cnn(CC(F)F)c5n4)C3=O)C2)cc(C(F)(F)F)n1] | 523 |
| 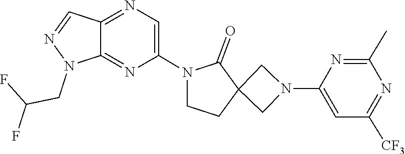 [SMILES: Cc1nc(N2CC3(CCN(c4cnc5cnn(CC(F)F)c5n4)C3=O)C2)cc(C(F)(F)F)n1] | 524 |
| 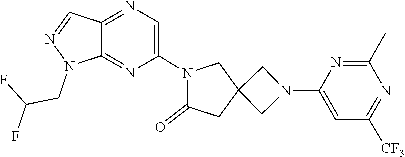 [SMILES: Cc1nc(N2CC3(CC(=O)N(c4cnc5cnn(CC(F)F)c5n4)C3)C2)cc(C(F)(F)F)n1] | 525 |
| 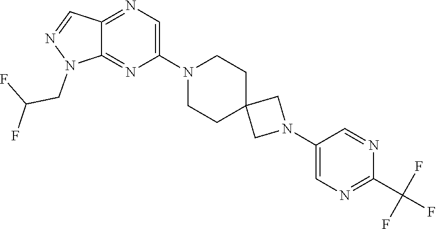 [SMILES: FC(F)Cn1ncc2ncc(N3CCC4(CC3)CN(c3cnc(C(F)(F)F)nc3)C4)nc21] | 526 |
| 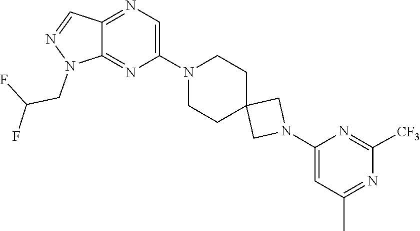 [SMILES: Cc1cc(N2CC3(CCN(c4cnc5cnn(CC(F)F)c5n4)CC3)C2)nc(C(F)(F)F)n1] | 527 |
| 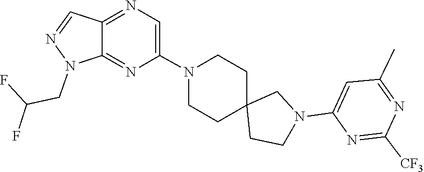 [SMILES: Cc1cc(N2CCC3(CCN(c4cnc5cnn(CC(F)F)c5n4)CC3)C2)nc(C(F)(F)F)n1] | 528 |
| 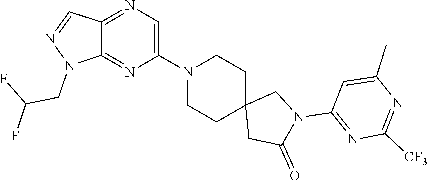 [SMILES: Cc1cc(N2CC3(CCN(c4cnc5cnn(CC(F)F)c5n4)CC3)CC2=O)nc(C(F)(F)F)n1] | 529 |
| 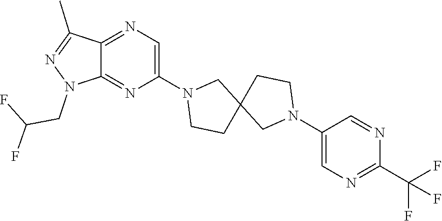 [SMILES: Cc1nn(CC(F)F)c2nc(N3CCC4(CCN(c5cnc(C(F)(F)F)nc5)C4)C3)cnc12] | 530 |
| 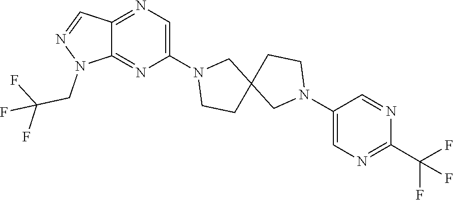 [SMILES: FC(F)(F)Cn1ncc2ncc(N3CCC4(CCN(c5cnc(C(F)(F)F)nc5)C4)C3)nc21] | 531 |
| 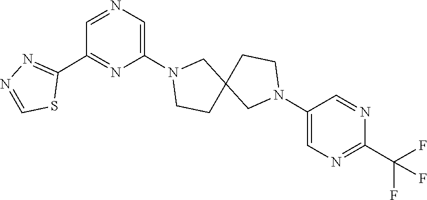 [SMILES: FC(F)(F)c1ncc(N2CCC3(CCN(c4cncc(-c5nncs5)n4)C3)C2)cn1] | 532 |
| 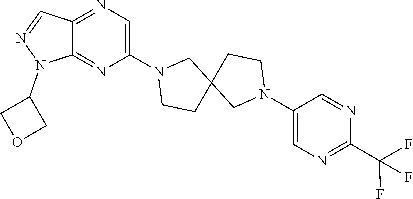 [SMILES: FC(F)(F)c1ncc(N2CCC3(CCN(c4cnc5cnn(C6COC6)c5n4)C3)C2)cn1] | 533 |
| 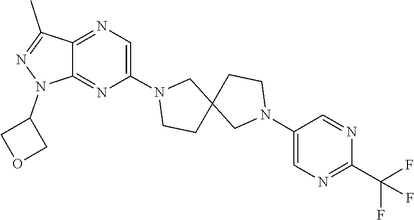 [SMILES: Cc1nn(C2COC2)c2nc(N3CCC4(CCN(c5cnc(C(F)(F)F)nc5)C4)C3)cnc12] | 534 |
| 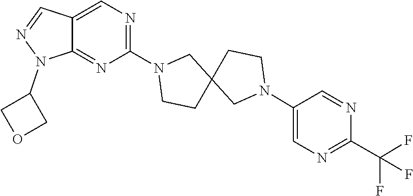 [SMILES: FC(F)(F)c1ncc(N2CCC3(CCN(c4ncc5cnn(C6COC6)c5n4)C3)C2)cn1] | 535 |
| 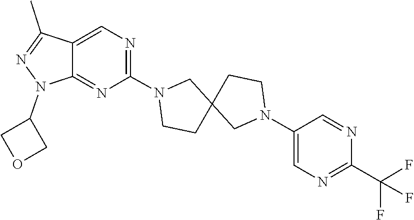 [SMILES: Cc1nn(C2COC2)c2nc(N3CCC4(CCN(c5cnc(C(F)(F)F)nc5)C4)C3)ncc12] | 536 |
| 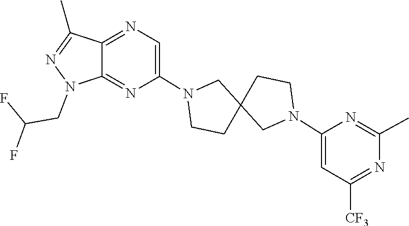 [SMILES: Cc1nc(N2CCC3(CCN(c4cnc5c(C)nn(CC(F)F)c5n4)C3)C2)cc(C(F)(F)F)n1] | 537 |
| 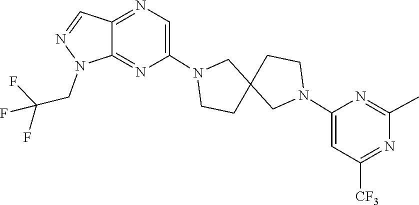 [SMILES: Cc1nc(N2CCC3(CCN(c4cnc5cnn(CC(F)(F)F)c5n4)C3)C2)cc(C(F)(F)F)n1] | 538 |
| 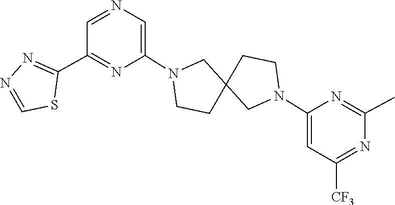 [SMILES: Cc1nc(N2CCC3(CCN(c4cncc(-c5nncs5)n4)C3)C2)cc(C(F)(F)F)n1] | 539 |
| 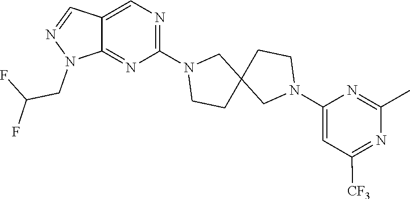 [SMILES: Cc1nc(N2CCC3(CCN(c4ncc5cnn(CC(F)F)c5n4)C3)C2)cc(C(F)(F)F)n1] | 540 |
| 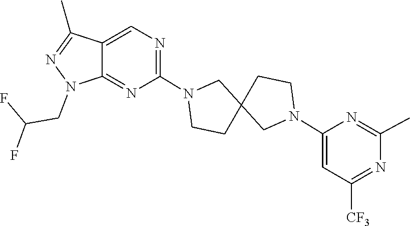 [SMILES: Cc1nc(N2CCC3(CCN(c4ncc5c(C)nn(CC(F)F)c5n4)C3)C2)cc(C(F)(F)F)n1] | 541 |
| 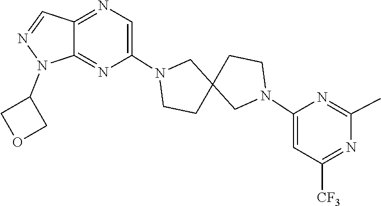 [SMILES: Cc1nc(N2CCC3(CCN(c4cnc5cnn(C6COC6)c5n4)C3)C2)cc(C(F)(F)F)n1] | 542 |
| 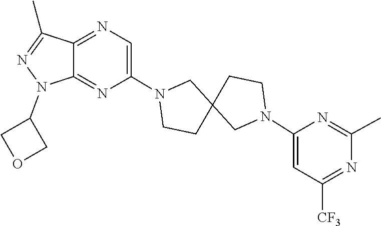 [SMILES: Cc1nc(N2CCC3(CCN(c4cnc5c(C)nn(C6COC6)c5n4)C3)C2)cc(C(F)(F)F)n1] | 543 |
| 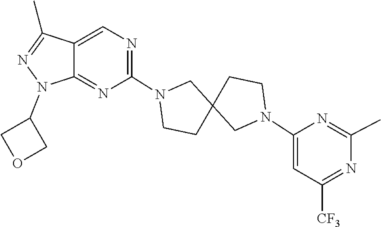 [SMILES: Cc1nc(N2CCC3(CCN(c4ncc5c(C)nn(C6COC6)c5n4)C3)C2)cc(C(F)(F)F)n1] | 544 |
| 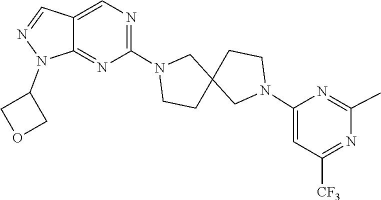 [SMILES: Cc1nc(N2CCC3(CCN(c4ncc5cnn(C6COC6)c5n4)C3)C2)cc(C(F)(F)F)n1] | 545 |
| 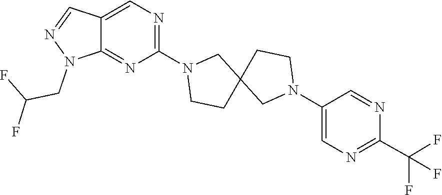 [SMILES: FC(F)Cn1ncc2cnc(N3CCC4(CCN(c5cnc(C(F)(F)F)nc5)C4)C3)nc21] | 546 |
| 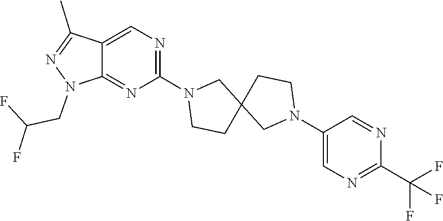 [SMILES: Cc1nn(CC(F)F)c2nc(N3CCC4(CCN(c5cnc(C(F)(F)F)nc5)C4)C3)ncc12] | 547 |
| 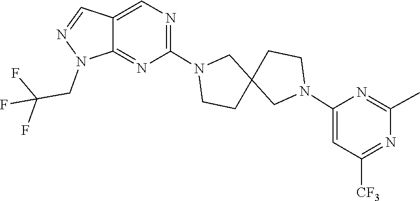 [SMILES: Cc1nc(N2CCC3(CCN(c4ncc5cnn(CC(F)(F)F)c5n4)C3)C2)cc(C(F)(F)F)n1] | 548 |
| 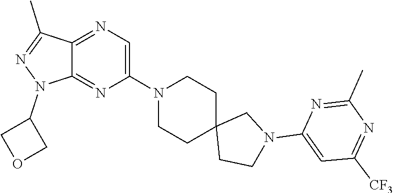 [SMILES: Cc1nc(N2CCC3(CCN(c4cnc5c(C)nn(C6COC6)c5n4)CC3)C2)cc(C(F)(F)F)n1] | 549 |
| 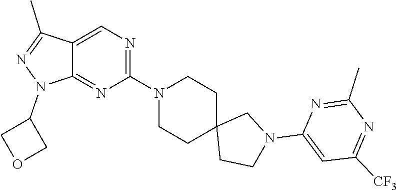 [SMILES: Cc1nc(N2CCC3(CCN(c4ncc5c(C)nn(C6COC6)c5n4)CC3)C2)cc(C(F)(F)F)n1] | 550 |
| 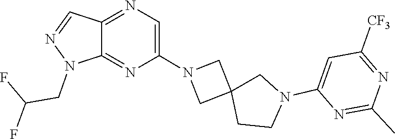 [SMILES: Cc1nc(N2CCC3(C2)CN(c2cnc4cnn(CC(F)F)c4n2)C3)cc(C(F)(F)F)n1] | 551 |
| 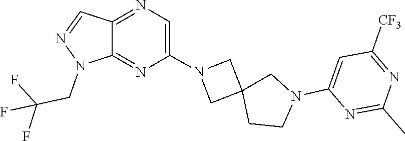 [SMILES: Cc1nc(N2CCC3(C2)CN(c2cnc4cnn(CC(F)(F)F)c4n2)C3)cc(C(F)(F)F)n1] | 552 |
| 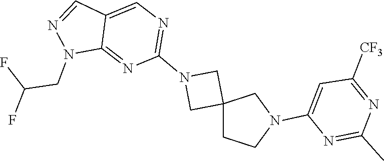 [SMILES: Cc1nc(N2CCC3(C2)CN(c2ncc4cnn(CC(F)F)c4n2)C3)cc(C(F)(F)F)n1] | 553 |
| 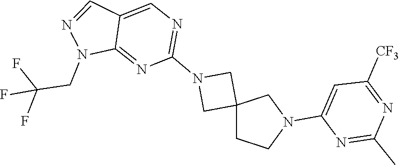 [SMILES: Cc1nc(N2CCC3(C2)CN(c2ncc4cnn(CC(F)(F)F)c4n2)C3)cc(C(F)(F)F)n1] | 554 |
| 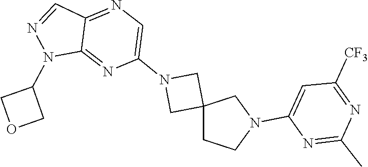 [SMILES: Cc1nc(N2CCC3(C2)CN(c2cnc4cnn(C5COC5)c4n2)C3)cc(C(F)(F)F)n1] | 555 |
| 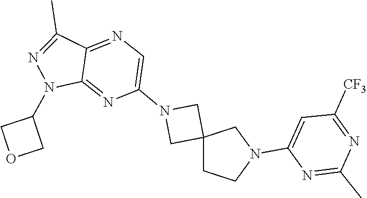 [SMILES: Cc1nc(N2CCC3(C2)CN(c2cnc4c(C)nn(C5COC5)c4n2)C3)cc(C(F)(F)F)n1] | 556 |
| 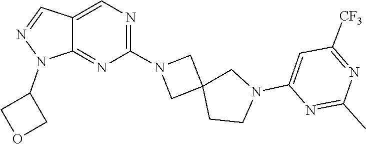 [SMILES: Cc1nc(N2CCC3(C2)CN(c2ncc4cnn(C5COC5)c4n2)C3)cc(C(F)(F)F)n1] | 557 |
| 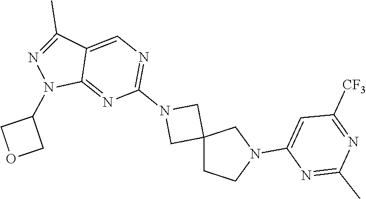 [SMILES: Cc1nc(N2CCC3(C2)CN(c2ncc4c(C)nn(C5COC5)c4n2)C3)cc(C(F)(F)F)n1] | 558 |
| 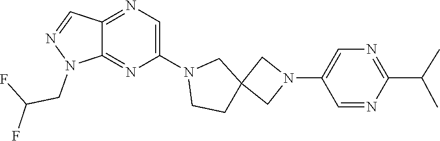 [SMILES: CC(C)c1ncc(N2CC3(CCN(c4cnc5cnn(CC(F)F)c5n4)C3)C2)cn1] | 559 |
| 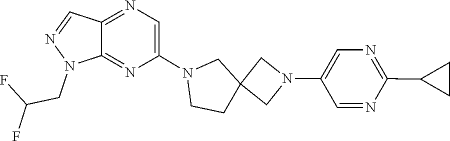 [SMILES: FC(F)Cn1ncc2ncc(N3CCC4(CN(c5cnc(C6CC6)nc5)C4)C3)nc21] | 560 |
| 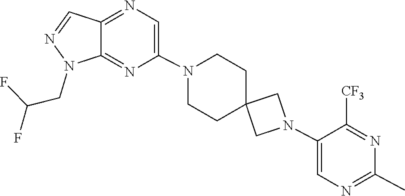 [SMILES: Cc1ncc(N2CC3(CCN(c4cnc5cnn(CC(F)F)c5n4)CC3)C2)c(C(F)(F)F)n1] | 561 |
| 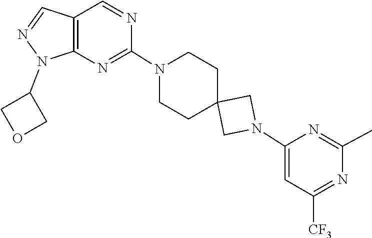 [SMILES: Cc1nc(N2CC3(CCN(c4ncc5cnn(C6COC6)c5n4)CC3)C2)cc(C(F)(F)F)n1] | 562 |
| 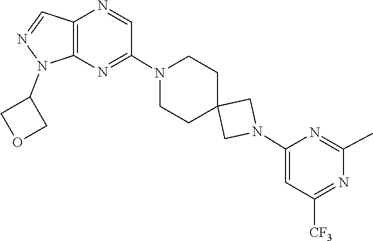 [SMILES: Cc1nc(N2CC3(CCN(c4cnc5cnn(C6COC6)c5n4)CC3)C2)cc(C(F)(F)F)n1] | 563 |
| 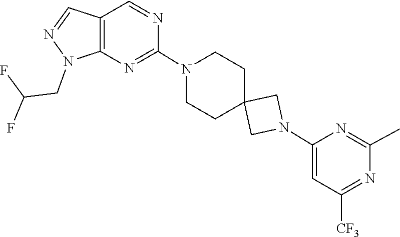 [SMILES: Cc1nc(N2CC3(CCN(c4ncc5cnn(CC(F)F)c5n4)CC3)C2)cc(C(F)(F)F)n1] | 564 |
| 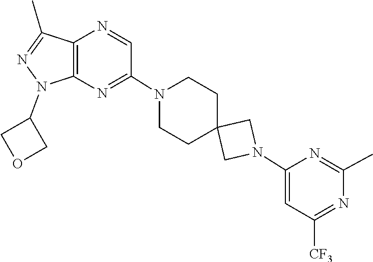 [SMILES: Cc1nc(N2CC3(CCN(c4cnc5c(C)nn(C6COC6)c5n4)CC3)C2)cc(C(F)(F)F)n1] | 565 |
| 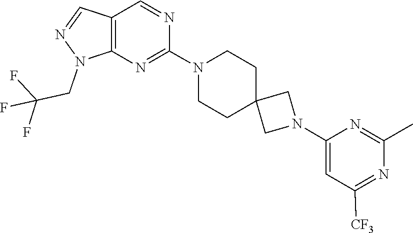 [SMILES: Cc1nc(N2CC3(CCN(c4ncc5cnn(CC(F)(F)F)c5n4)CC3)C2)cc(C(F)(F)F)n1] | 566 |
| 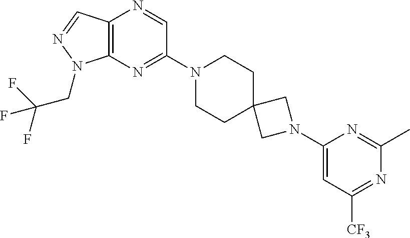 [SMILES: Cc1nc(N2CC3(CCN(c4cnc5cnn(CC(F)(F)F)c5n4)CC3)C2)cc(C(F)(F)F)n1] | 567 |
| 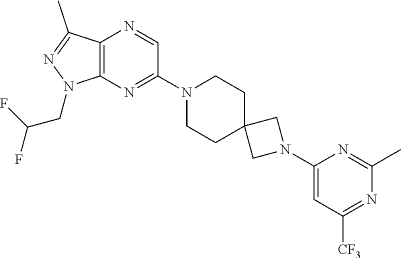 [SMILES: Cc1nc(N2CC3(CCN(c4cnc5c(C)nn(CC(F)F)c5n4)CC3)C2)cc(C(F)(F)F)n1] | 568 |
| 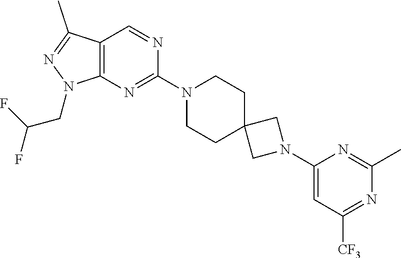 [SMILES: Cc1nc(N2CC3(CCN(c4ncc5c(C)nn(CC(F)F)c5n4)CC3)C2)cc(C(F)(F)F)n1] | 569 |
| 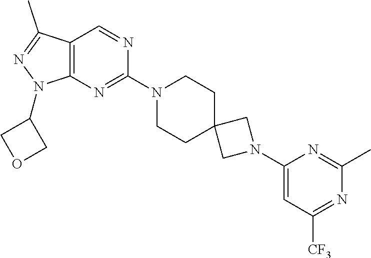 [SMILES: Cc1nc(N2CC3(CCN(c4ncc5c(C)nn(C6COC6)c5n4)CC3)C2)cc(C(F)(F)F)n1] | 570 |
| 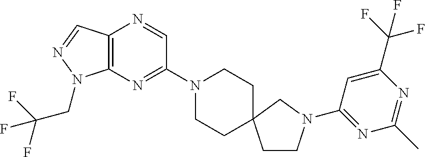 [SMILES: Cc1nc(N2CCC3(CCN(c4cnc5cnn(CC(F)(F)F)c5n4)CC3)C2)cc(C(F)(F)F)n1] | 571 |
| 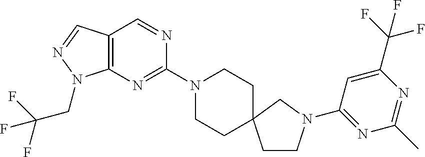 [SMILES: Cc1nc(N2CCC3(CCN(c4ncc5cnn(CC(F)(F)F)c5n4)CC3)C2)cc(C(F)(F)F)n1] | 572 |
| 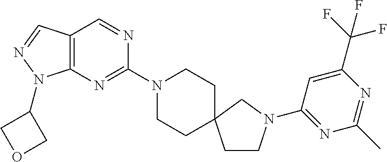 [SMILES: Cc1nc(N2CCC3(CCN(c4ncc5cnn(C6COC6)c5n4)CC3)C2)cc(C(F)(F)F)n1] | 573 |
| 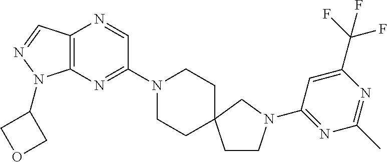 [SMILES: Cc1nc(N2CCC3(CCN(c4cnc5cnn(C6COC6)c5n4)CC3)C2)cc(C(F)(F)F)n1] | 574 |
| 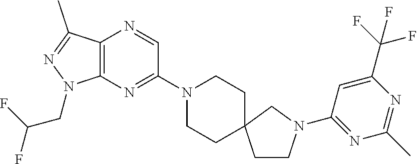 [SMILES: Cc1nc(N2CCC3(CCN(c4cnc5c(C)nn(CC(F)F)c5n4)CC3)C2)cc(C(F)(F)F)n1] | 575 |
| 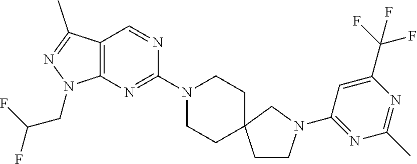 [SMILES: Cc1nc(N2CCC3(CCN(c4ncc5c(C)nn(CC(F)F)c5n4)CC3)C2)cc(C(F)(F)F)n1] | 576 |
| 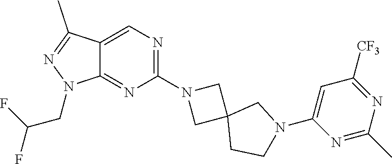 [SMILES: Cc1nc(N2CCC3(C2)CN(c2ncc4c(C)nn(CC(F)F)c4n2)C3)cc(C(F)(F)F)n1] | 577 |
| 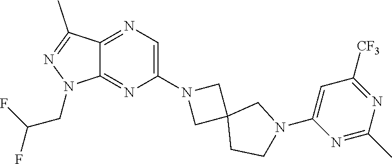 [SMILES: Cc1nc(N2CCC3(C2)CN(c2cnc4c(C)nn(CC(F)F)c4n2)C3)cc(C(F)(F)F)n1] | 578 |
| 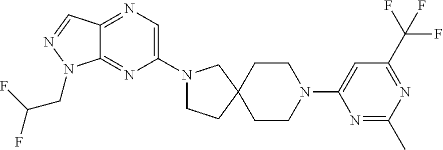 [SMILES: Cc1nc(N2CCC3(CC2)CCN(c2cnc4cnn(CC(F)F)c4n2)C3)cc(C(F)(F)F)n1] | 579 |
| 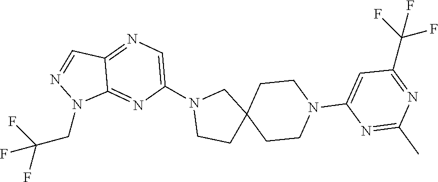 [SMILES: Cc1nc(N2CCC3(CC2)CCN(c2cnc4cnn(CC(F)(F)F)c4n2)C3)cc(C(F)(F)F)n1] | 580 |
| 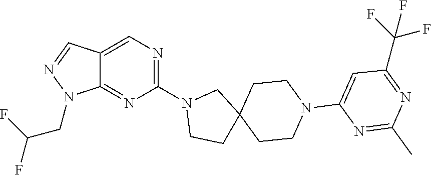 [SMILES: Cc1nc(N2CCC3(CC2)CCN(c2ncc4cnn(CC(F)F)c4n2)C3)cc(C(F)(F)F)n1] | 581 |
| 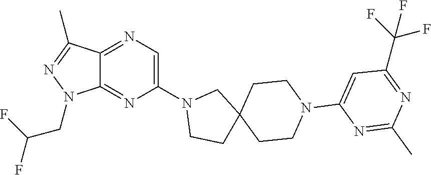 [SMILES: Cc1nc(N2CCC3(CC2)CCN(c2cnc4c(C)nn(CC(F)F)c4n2)C3)cc(C(F)(F)F)n1] | 582 |
| 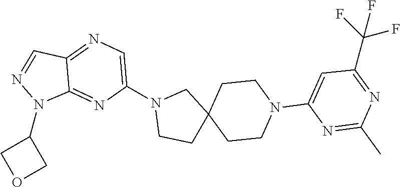 [SMILES: Cc1nc(N2CCC3(CC2)CCN(c2cnc4cnn(C5COC5)c4n2)C3)cc(C(F)(F)F)n1] | 583 |
| 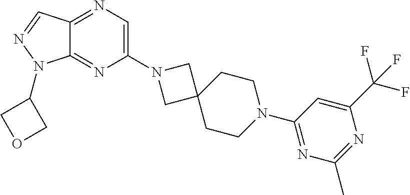 [SMILES: Cc1nc(N2CCC3(CC2)CN(c2cnc4cnn(C5COC5)c4n2)C3)cc(C(F)(F)F)n1] | 584 |
| 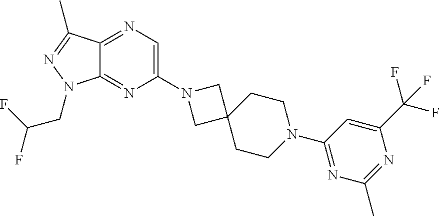 [SMILES: Cc1nc(N2CCC3(CC2)CN(c2cnc4c(C)nn(CC(F)F)c4n2)C3)cc(C(F)(F)F)n1] | 585 |
| 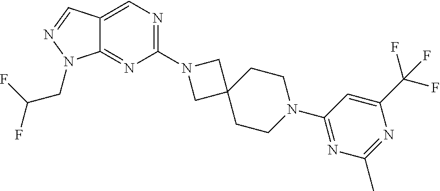 [SMILES: Cc1nc(N2CCC3(CC2)CN(c2ncc4cnn(CC(F)F)c4n2)C3)cc(C(F)(F)F)n1] | 586 |
| 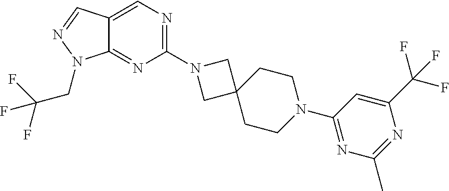 [SMILES: Cc1nc(N2CCC3(CC2)CN(c2ncc4cnn(CC(F)(F)F)c4n2)C3)cc(C(F)(F)F)n1] | 587 |
| 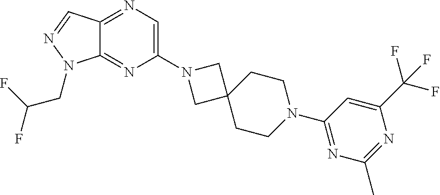 [SMILES: Cc1nc(N2CCC3(CC2)CN(c2cnc4cnn(CC(F)F)c4n2)C3)cc(C(F)(F)F)n1] | 588 |
| 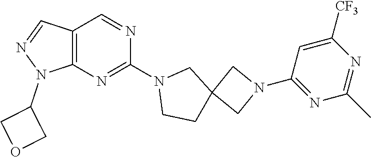 [SMILES: Cc1nc(N2CC3(CCN(c4ncc5cnn(C6COC6)c5n4)C3)C2)cc(C(F)(F)F)n1] | 589 |
| 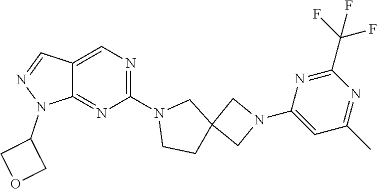 [SMILES: Cc1cc(N2CC3(CCN(c4ncc5cnn(C6COC6)c5n4)C3)C2)nc(C(F)(F)F)n1] | 590 |
| 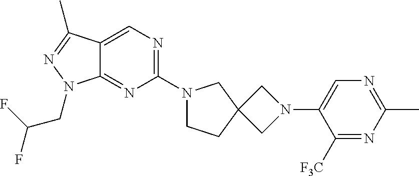 [SMILES: Cc1ncc(N2CC3(CCN(c4ncc5c(C)nn(CC(F)F)c5n4)C3)C2)c(C(F)(F)F)n1] | 591 |
| 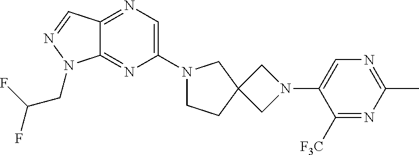 [SMILES: Cc1ncc(N2CC3(CCN(c4cnc5cnn(CC(F)F)c5n4)C3)C2)c(C(F)(F)F)n1] | 592 |
| 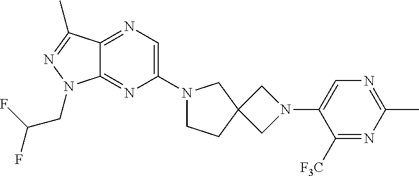 [SMILES: Cc1ncc(N2CC3(CCN(c4cnc5c(C)nn(CC(F)F)c5n4)C3)C2)c(C(F)(F)F)n1] | 593 |
| 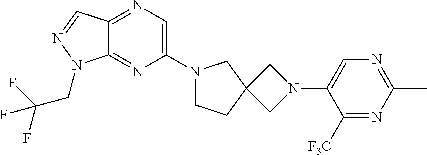 [SMILES: Cc1ncc(N2CC3(CCN(c4cnc5cnn(CC(F)(F)F)c5n4)C3)C2)c(C(F)(F)F)n1] | 594 |
| 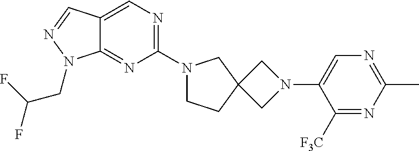 [SMILES: Cc1ncc(N2CC3(CCN(c4ncc5cnn(CC(F)F)c5n4)C3)C2)c(C(F)(F)F)n1] | 595 |
| 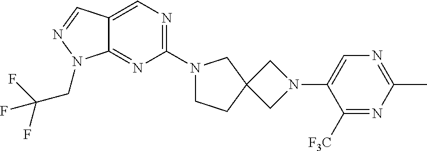 [SMILES: Cc1ncc(N2CC3(CCN(c4ncc5cnn(CC(F)(F)F)c5n4)C3)C2)c(C(F)(F)F)n1] | 596 |
| 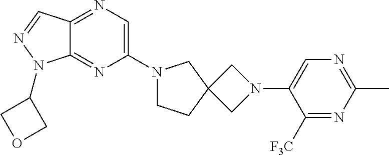 [SMILES: Cc1ncc(N2CC3(CCN(c4cnc5cnn(C6COC6)c5n4)C3)C2)c(C(F)(F)F)n1] | 597 |
| 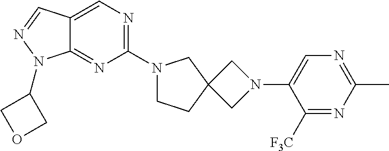 [SMILES: Cc1ncc(N2CC3(CCN(c4ncc5cnn(C6COC6)c5n4)C3)C2)c(C(F)(F)F)n1] | 598 |
| 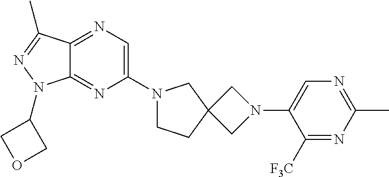 [SMILES: Cc1ncc(N2CC3(CCN(c4cnc5c(C)nn(C6COC6)c5n4)C3)C2)c(C(F)(F)F)n1] | 599 |
| 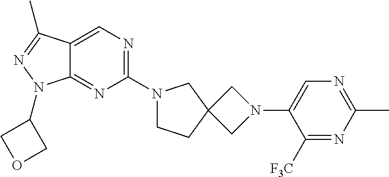 [SMILES: Cc1ncc(N2CC3(CCN(c4ncc5c(C)nn(C6COC6)c5n4)C3)C2)c(C(F)(F)F)n1] | 600 |
| 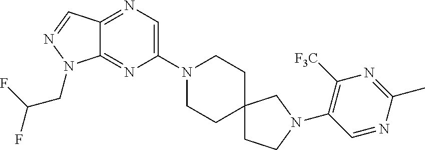 [SMILES: Cc1ncc(N2CCC3(CCN(c4cnc5cnn(CC(F)F)c5n4)CC3)C2)c(C(F)(F)F)n1] | 601 |
| 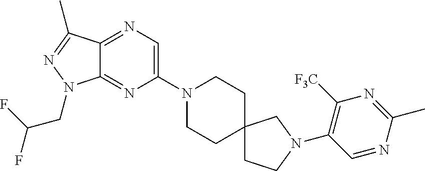 [SMILES: Cc1ncc(N2CCC3(CCN(c4cnc5c(C)nn(CC(F)F)c5n4)CC3)C2)c(C(F)(F)F)n1] | 602 |
| 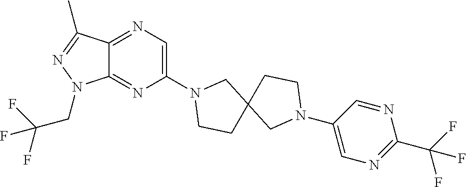 [SMILES: Cc1nn(CC(F)(F)F)c2nc(N3CCC4(CCN(c5cnc(C(F)(F)F)nc5)C4)C3)cnc12] | 603 |
| 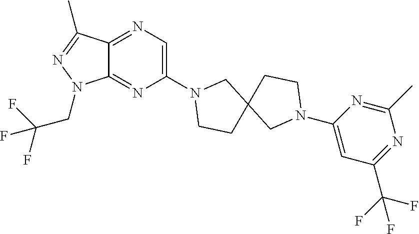 [SMILES: Cc1nc(N2CCC3(CCN(c4cnc5c(C)nn(CC(F)(F)F)c5n4)C3)C2)cc(C(F)(F)F)n1] | 604 |
| 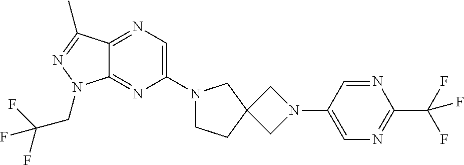 [SMILES: Cc1nn(CC(F)(F)F)c2nc(N3CCC4(CN(c5cnc(C(F)(F)F)nc5)C4)C3)cnc12] | 605 |
| 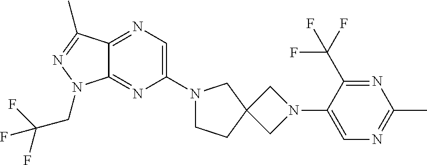 [SMILES: Cc1ncc(N2CC3(CCN(c4cnc5c(C)nn(CC(F)(F)F)c5n4)C3)C2)c(C(F)(F)F)n1] | 606 |
| 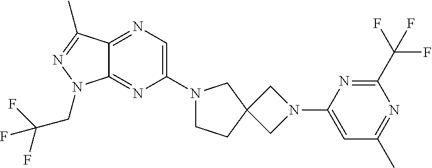 [SMILES: Cc1cc(N2CC3(CCN(c4cnc5c(C)nn(CC(F)(F)F)c5n4)C3)C2)nc(C(F)(F)F)n1] | 607 |
| 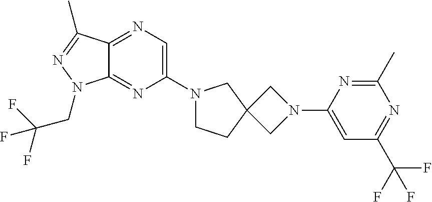 [SMILES: Cc1nc(N2CC3(CCN(c4cnc5c(C)nn(CC(F)(F)F)c5n4)C3)C2)cc(C(F)(F)F)n1] | 608 |
| 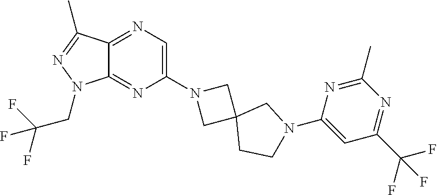 [SMILES: Cc1nc(N2CCC3(C2)CN(c2cnc4c(C)nn(CC(F)(F)F)c4n2)C3)cc(C(F)(F)F)n1] | 609 |
| 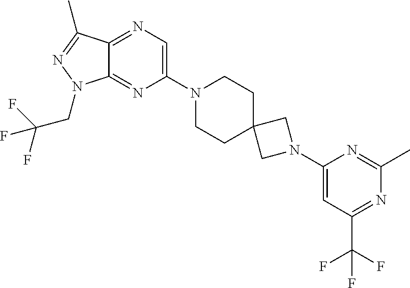 [SMILES: Cc1nc(N2CC3(CCN(c4cnc5c(C)nn(CC(F)(F)F)c5n4)CC3)C2)cc(C(F)(F)F)n1] | 610 |
| 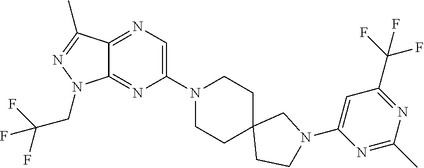 [SMILES: Cc1nc(N2CCC3(CCN(c4cnc5c(C)nn(CC(F)(F)F)c5n4)CC3)C2)cc(C(F)(F)F)n1] | 611 |
| 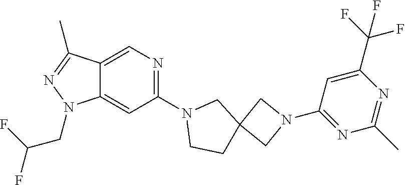 [SMILES: Cc1nc(N2CC3(CCN(c4cc5c(cn4)c(C)nn5CC(F)F)C3)C2)cc(C(F)(F)F)n1] | 612 |
| 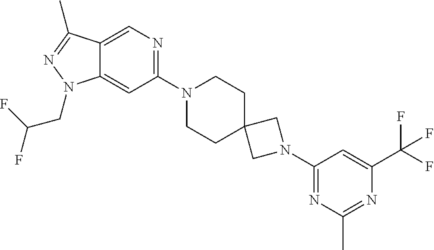 [SMILES: Cc1nc(N2CC3(CCN(c4cc5c(cn4)c(C)nn5CC(F)F)CC3)C2)cc(C(F)(F)F)n1] | 613 |
| 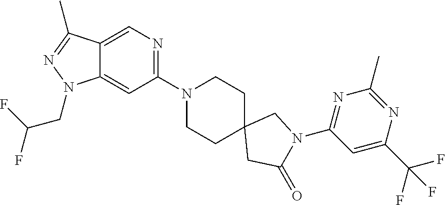 [SMILES: Cc1nc(N2CC3(CCN(c4cc5c(cn4)c(C)nn5CC(F)F)CC3)CC2=O)cc(C(F)(F)F)n1] | 614 |
| 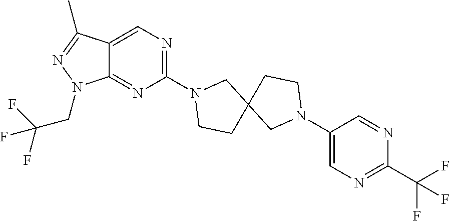 [SMILES: Cc1nn(CC(F)(F)F)c2nc(N3CCC4(CCN(c5cnc(C(F)(F)F)nc5)C4)C3)ncc12] | 615 |
| 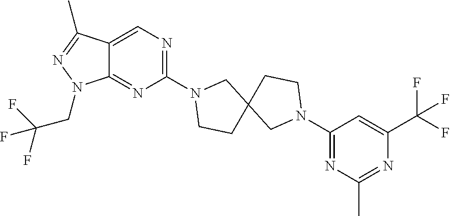 [SMILES: Cc1nc(N2CCC3(CCN(c4ncc5c(C)nn(CC(F)(F)F)c5n4)C3)C2)cc(C(F)(F)F)n1] | 616 |
| 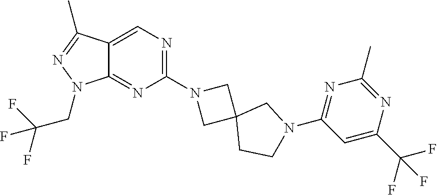 [SMILES: Cc1nc(N2CCC3(C2)CN(c2ncc4c(C)nn(CC(F)(F)F)c4n2)C3)cc(C(F)(F)F)n1] | 617 |
| 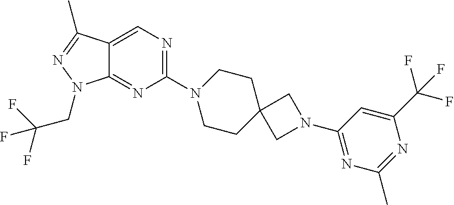 [SMILES: Cc1nc(N2CC3(CCN(c4ncc5c(C)nn(CC(F)(F)F)c5n4)CC3)C2)cc(C(F)(F)F)n1] | 618 |
| 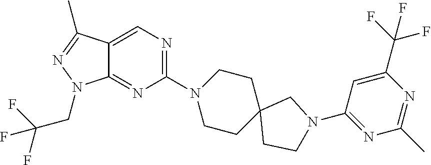 [SMILES: Cc1nc(N2CCC3(CCN(c4ncc5c(C)nn(CC(F)(F)F)c5n4)CC3)C2)cc(C(F)(F)F)n1] | 619 |
| 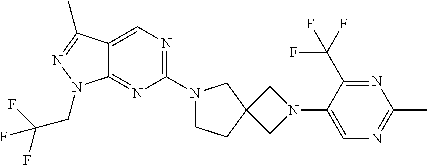 [SMILES: Cc1ncc(N2CC3(CCN(c4ncc5c(C)nn(CC(F)(F)F)c5n4)C3)C2)c(C(F)(F)F)n1] | 620 |
| 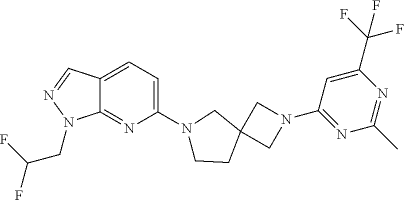 [SMILES: Cc1nc(N2CC3(CCN(c4ccc5cnn(CC(F)F)c5n4)C3)C2)cc(C(F)(F)F)n1] | 621 |
| 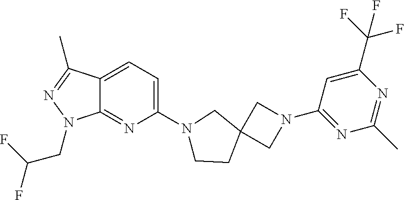 [SMILES: Cc1nc(N2CC3(CCN(c4ccc5c(C)nn(CC(F)F)c5n4)C3)C2)cc(C(F)(F)F)n1] | 622 |
| 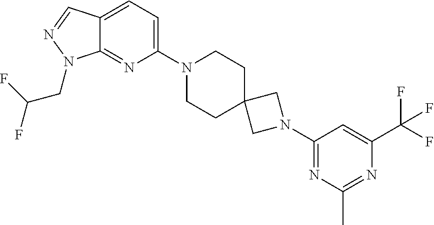 [SMILES: Cc1nc(N2CC3(CCN(c4ccc5cnn(CC(F)F)c5n4)CC3)C2)cc(C(F)(F)F)n1] | 623 |
| 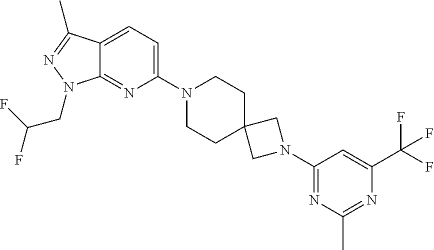 [SMILES: Cc1nc(N2CC3(CCN(c4ccc5c(C)nn(CC(F)F)c5n4)CC3)C2)cc(C(F)(F)F)n1] | 624 |
| 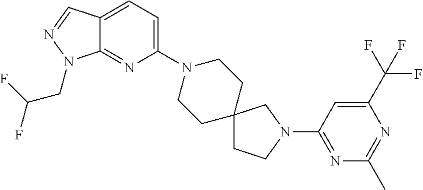 [SMILES: Cc1nc(N2CCC3(CCN(c4ccc5cnn(CC(F)F)c5n4)CC3)C2)cc(C(F)(F)F)n1] | 625 |
| 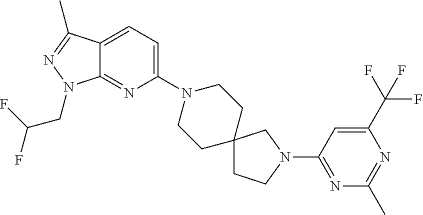 [SMILES: Cc1nc(N2CCC3(CCN(c4ccc5c(C)nn(CC(F)F)c5n4)CC3)C2)cc(C(F)(F)F)n1] | 626 |
| 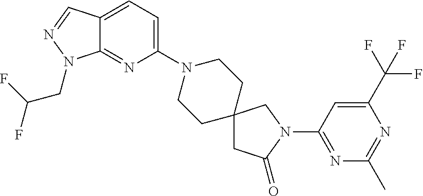 [SMILES: Cc1nc(N2CC3(CCN(c4ccc5cnn(CC(F)F)c5n4)CC3)CC2=O)cc(C(F)(F)F)n1] | 627 |
| 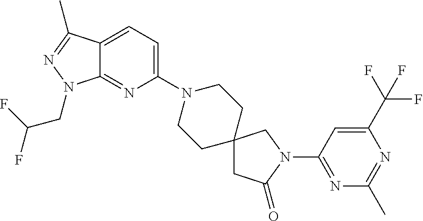 [SMILES: Cc1nc(N2CC3(CCN(c4ccc5c(C)nn(CC(F)F)c5n4)CC3)CC2=O)cc(C(F)(F)F)n1] | 628 |
| 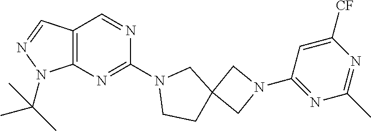 | 629 |
| 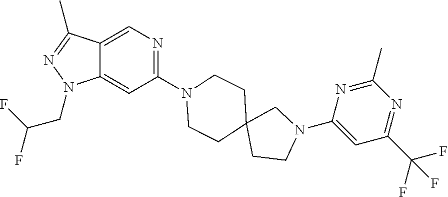 [SMILES: Cc1nc(N2CCC3(CCN(c4cc5c(cn4)c(C)nn5CC(F)F)CC3)C2)cc(C(F)(F)F)n1] | 630 |
| 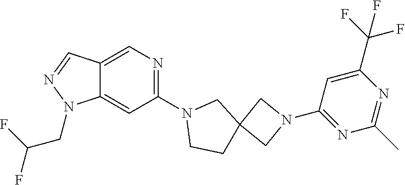 [SMILES: Cc1nc(N2CC3(CCN(c4cc5c(cn4)cnn5CC(F)F)C3)C2)cc(C(F)(F)F)n1] | 631 |
| 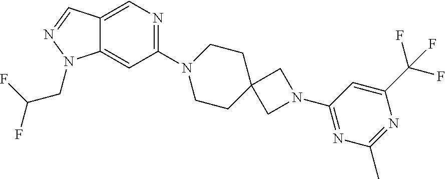 [SMILES: Cc1nc(N2CC3(CCN(c4cc5c(cn4)cnn5CC(F)F)CC3)C2)cc(C(F)(F)F)n1] | 632 |
| 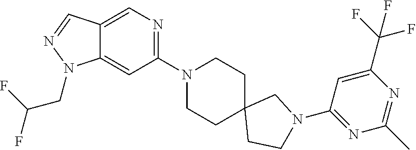 [SMILES: Cc1nc(N2CCC3(CCN(c4cc5c(cn4)cnn5CC(F)F)CC3)C2)cc(C(F)(F)F)n1] | 633 |
| 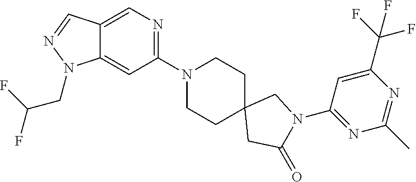 [SMILES: Cc1nc(N2CC3(CCN(c4cc5c(cn4)cnn5CC(F)F)CC3)CC2=O)cc(C(F)(F)F)n1] | 634 |
| 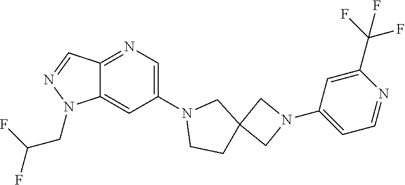 [SMILES: FC(F)Cn1ncc2ncc(N3CCC4(C3)CN(c3ccnc(C(F)(F)F)c3)C4)cc21] | 635 |
| 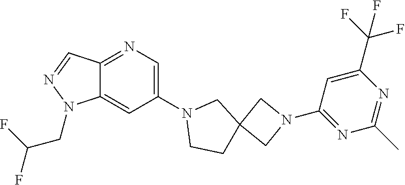 [SMILES: Cc1nc(N2CC3(CCN(c4cnc5cnn(CC(F)F)c5c4)C3)C2)cc(C(F)(F)F)n1] | 636 |
| 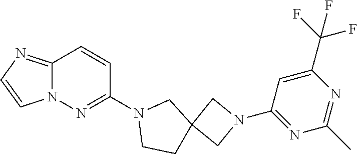 [SMILES: Cc1nc(N2CC3(CCN(c4ccc5nccn5n4)C3)C2)cc(C(F)(F)F)n1] | 637 |
| 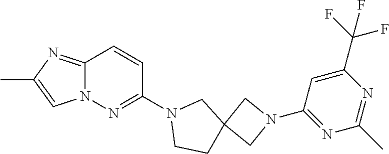 [SMILES: Cc1cn2nc(N3CCC4(CN(c5cc(C(F)(F)F)nc(C)n5)C4)C3)ccc2n1] | 638 |
| 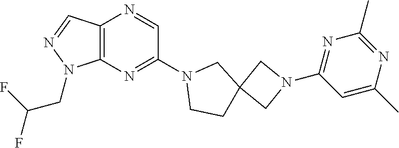 [SMILES: Cc1cc(N2CC3(CCN(c4cnc5cnn(CC(F)F)c5n4)C3)C2)nc(C)n1] | 639 |
| 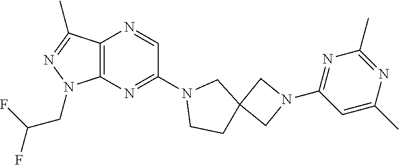 [SMILES: Cc1cc(N2CC3(CCN(c4cnc5c(C)nn(CC(F)F)c5n4)C3)C2)nc(C)n1] | 640 |
| 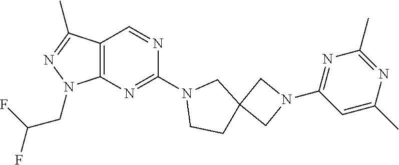 [SMILES: Cc1cc(N2CC3(CCN(c4ncc5c(C)nn(CC(F)F)c5n4)C3)C2)nc(C)n1] | 641 |
| 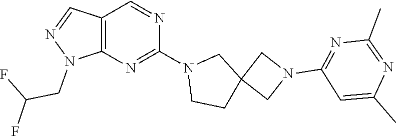 [SMILES: Cc1cc(N2CC3(CCN(c4ncc5cnn(CC(F)F)c5n4)C3)C2)nc(C)n1] | 642 |
| 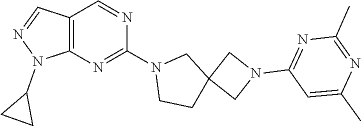 [SMILES: Cc1cc(N2CC3(CCN(c4ncc5cnn(C6CC6)c5n4)C3)C2)nc(C)n1] | 643 |
| 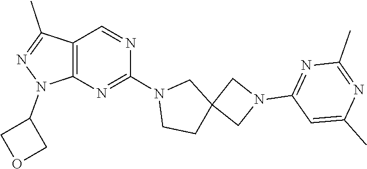 [SMILES: Cc1cc(N2CC3(CCN(c4ncc5c(C)nn(C6COC6)c5n4)C3)C2)nc(C)n1] | 644 |
| 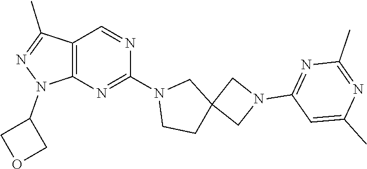 [SMILES: Cc1cc(N2CC3(CCN(c4ncc5c(C)nn(C6COC6)c5n4)C3)C2)nc(C)n1] | 645 |
| 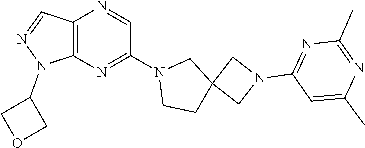 [SMILES: Cc1cc(N2CC3(CCN(c4cnc5cnn(C6COC6)c5n4)C3)C2)nc(C)n1] | 646 |
| 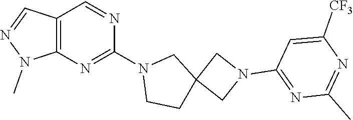 [SMILES: Cc1nc(N2CC3(CCN(c4ncc5cnn(C)c5n4)C3)C2)cc(C(F)(F)F)n1] | 647 |
| 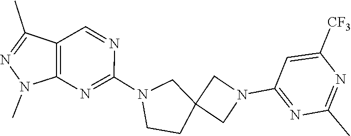 [SMILES: Cc1nc(N2CC3(CCN(c4ncc5c(C)nn(C)c5n4)C3)C2)cc(C(F)(F)F)n1] | 648 |
| 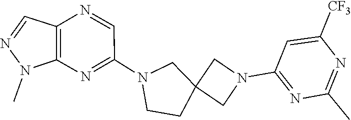 [SMILES: Cc1nc(N2CC3(CCN(c4cnc5cnn(C)c5n4)C3)C2)cc(C(F)(F)F)n1] | 649 |
| 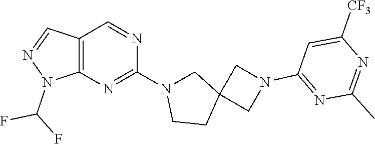 [SMILES: Cc1nc(N2CC3(CCN(c4ncc5cnn(C(F)F)c5n4)C3)C2)cc(C(F)(F)F)n1] | 650 |
| 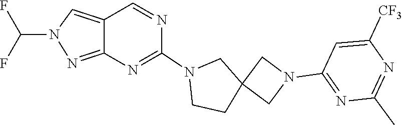 [SMILES: Cc1nc(N2CC3(CCN(c4ncc5cn(C(F)F)nc5n4)C3)C2)cc(C(F)(F)F)n1] | 651 |
| 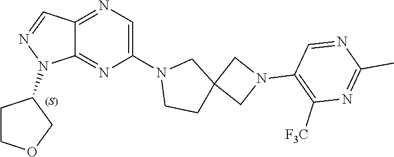 | 652 |
| 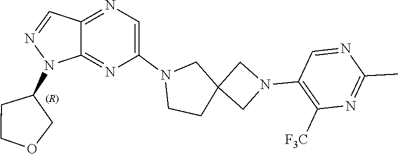 | 653 |
| 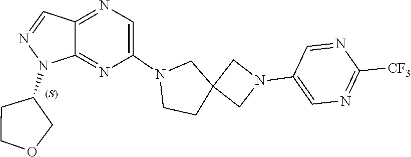 | 654 |
| 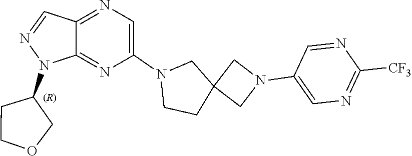 | 655 |
| 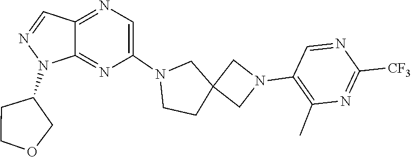 [SMILES: Cc1nc(C(F)(F)F)ncc1N1CC2(CCN(c3cnc4cnn([C@H]5CCOC5)c4n3)C2)C1] | 656 |
| 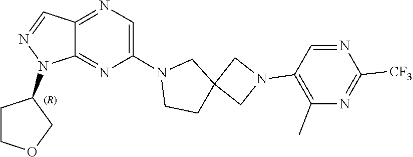 | 657 |
| 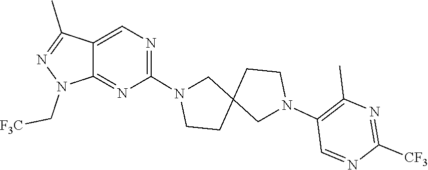 [SMILES: Cc1nc(C(F)(F)F)ncc1N1CCC2(CCN(c3ncc4c(C)nn(CC(F)(F)F)c4n3)C2)C1] | 658 |
| 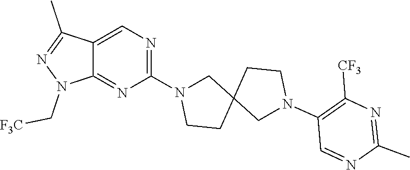 [SMILES: Cc1ncc(N2CCC3(CCN(c4ncc5c(C)nn(CC(F)(F)F)c5n4)C3)C2)c(C(F)(F)F)n1] | 659 |
| 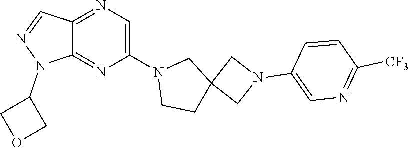 [SMILES: FC(F)(F)c1ccc(N2CC3(CCN(c4cnc5cnn(C6COC6)c5n4)C3)C2)cn1] | 660 |
| 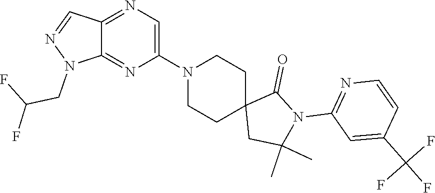 [SMILES: CC1(C)CC2(CCN(c3cnc4cnn(CC(F)F)c4n3)CC2)C(=O)N1c1cc(C(F)(F)F)ccn1] | 661 |
| 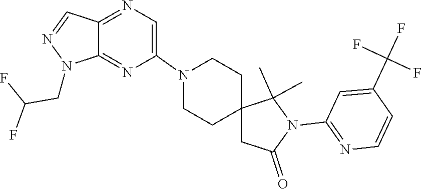 [SMILES: CC1(C)N(c2cc(C(F)(F)F)ccn2)C(=O)CC12CCN(c1cnc3cnn(CC(F)F)c3n1)CC2] | 662 |
| 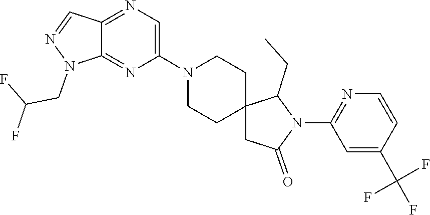 [SMILES: CCC1N(c2cc(C(F)(F)F)ccn2)C(=O)CC12CCN(c1cnc3cnn(CC(F)F)c3n1)CC2] | 663 |
| 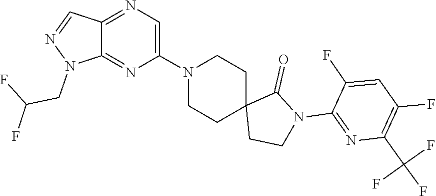 [SMILES: O=C1N(c2nc(C(F)(F)F)c(F)cc2F)CCC12CCN(c1cnc3cnn(CC(F)F)c3n1)CC2] | 664 |
| 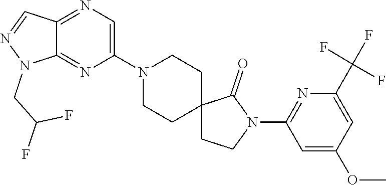 [SMILES: COc1cc(N2CCC3(CCN(c4cnc5cnn(CC(F)F)c5n4)CC3)C2=O)nc(C(F)(F)F)c1] | 665 |
| 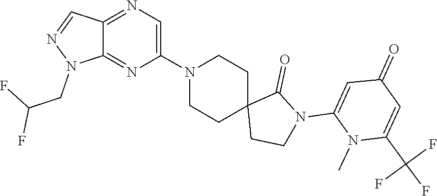 [SMILES: Cn1c(N2CCC3(CCN(c4cnc5cnn(CC(F)F)c5n4)CC3)C2=O)cc(=O)cc1C(F)(F)F] | 666 |
| 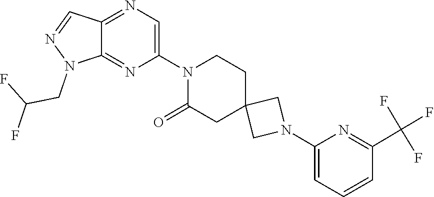 [SMILES: O=C1CC2(CCN1c1cnc3cnn(CC(F)F)c3n1)CN(c1cccc(C(F)(F)F)n1)C2] | 667 |
| 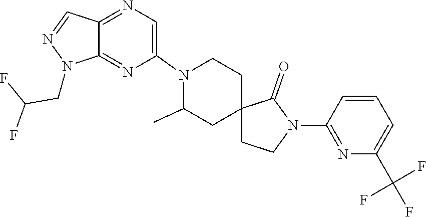 [SMILES: CC1CC2(CCN(c3cccc(C(F)(F)F)n3)C2=O)CCN1c1cnc2cnn(CC(F)F)c2n1] | 668 |
| 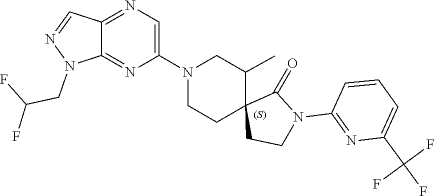 | 669 |
| 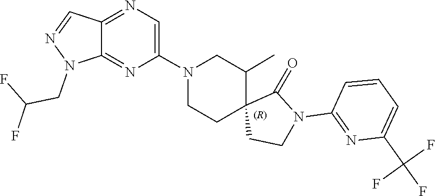 | 670 |
| 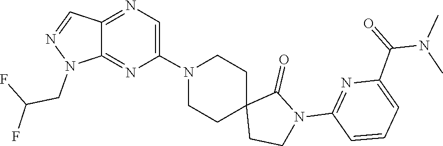 [SMILES: CN(C)C(=O)c1cccc(N2CCC3(CCN(c4cnc5cnn(CC(F)F)c5n4)CC3)C2=O)n1] | 671 |
| 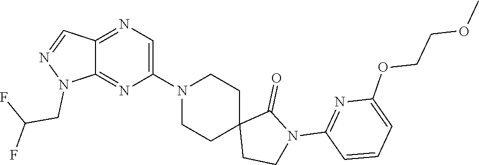 [SMILES: COCCOc1cccc(N2CCC3(CCN(c4cnc5cnn(CC(F)F)c5n4)CC3)C2=O)n1] | 672 |
| 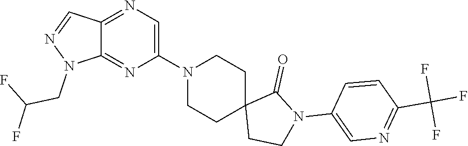 [SMILES: O=C1N(c2ccc(C(F)(F)F)nc2)CCC12CCN(c1cnc3cnn(CC(F)F)c3n1)CC2] | 673 |
| 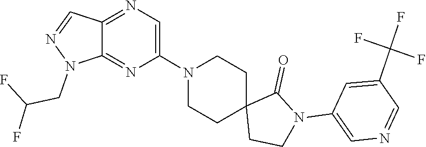 [SMILES: O=C1N(c2cncc(C(F)(F)F)c2)CCC12CCN(c1cnc3cnn(CC(F)F)c3n1)CC2] | 674 |
| 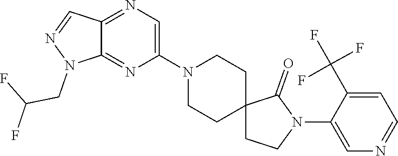 [SMILES: O=C1N(c2cnccc2C(F)(F)F)CCC12CCN(c1cnc3cnn(CC(F)F)c3n1)CC2] | 675 |
| 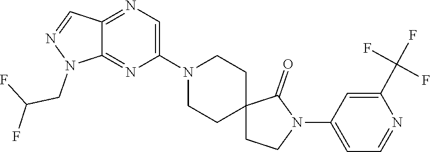 [SMILES: O=C1N(c2ccnc(C(F)(F)F)c2)CCC12CCN(c1cnc3cnn(CC(F)F)c3n1)CC2] | 676 |
| 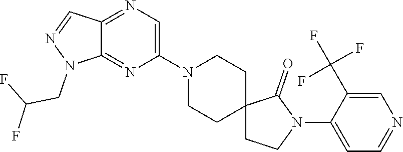 [SMILES: O=C1N(c2ccncc2C(F)(F)F)CCC12CCN(c1cnc3cnn(CC(F)F)c3n1)CC2] | 677 |
| 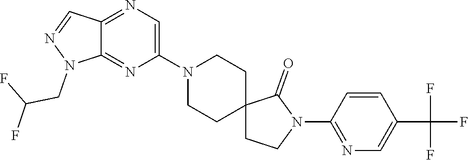 [SMILES: O=C1N(c2ccc(C(F)(F)F)cn2)CCC12CCN(c1cnc3cnn(CC(F)F)c3n1)CC2] | 678 |
| 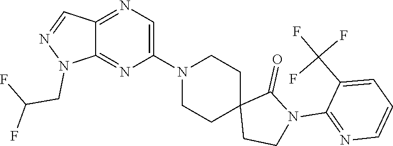 [SMILES: O=C1N(c2ncccc2C(F)(F)F)CCC12CCN(c1cnc3cnn(CC(F)F)c3n1)CC2] | 679 |
| 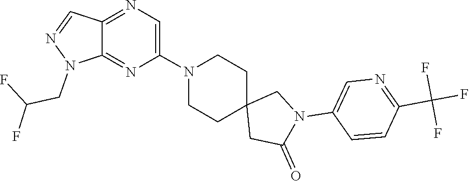 [SMILES: O=C1CC2(CCN(c3cnc4cnn(CC(F)F)c4n3)CC2)CN1c1ccc(C(F)(F)F)nc1] | 680 |
| 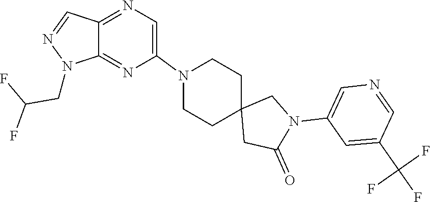 [SMILES: O=C1CC2(CCN(c3cnc4cnn(CC(F)F)c4n3)CC2)CN1c1cncc(C(F)(F)F)c1] | 681 |
| 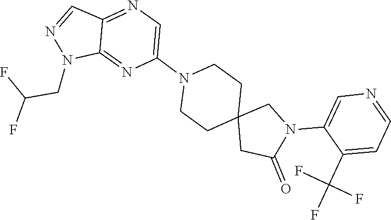 [SMILES: O=C1CC2(CCN(c3cnc4cnn(CC(F)F)c4n3)CC2)CN1c1cnccc1C(F)(F)F] | 682 |
| 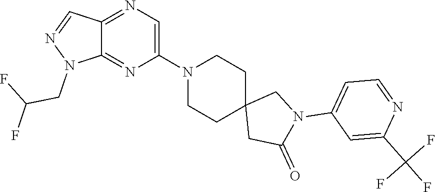 [SMILES: O=C1CC2(CCN(c3cnc4cnn(CC(F)F)c4n3)CC2)CN1c1ccnc(C(F)(F)F)c1] | 683 |
| 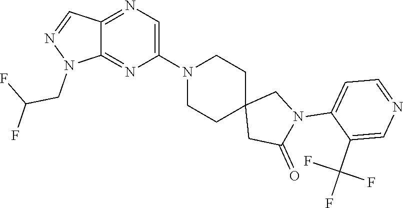 [SMILES: O=C1CC2(CCN(c3cnc4cnn(CC(F)F)c4n3)CC2)CN1c1ccncc1C(F)(F)F] | 684 |
| 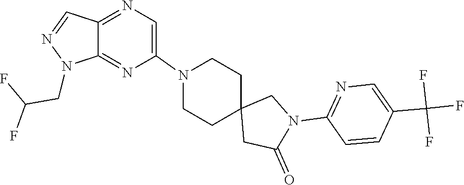 [SMILES: O=C1CC2(CCN(c3cnc4cnn(CC(F)F)c4n3)CC2)CN1c1ccc(C(F)(F)F)cn1] | 685 |
| 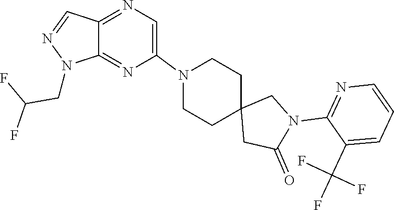 [SMILES: O=C1CC2(CCN(c3cnc4cnn(CC(F)F)c4n3)CC2)CN1c1ncccc1C(F)(F)F] | 686 |
| 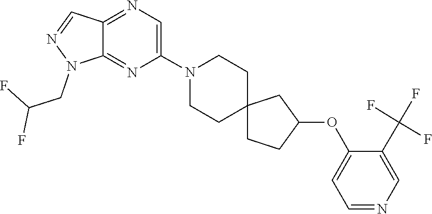 [SMILES: FC(F)Cn1ncc2ncc(N3CCC4(CCC(Oc5ccncc5C(F)(F)F)C4)CC3)nc21] | 687 |
| 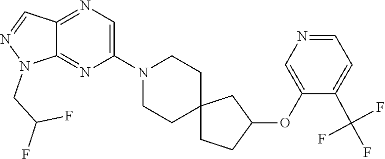 [SMILES: FC(F)Cn1ncc2ncc(N3CCC4(CCC(Oc5cnccc5C(F)(F)F)C4)CC3)nc21] | 688 |
| 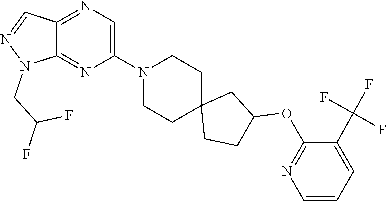 [SMILES: FC(F)Cn1ncc2ncc(N3CCC4(CCC(Oc5ncccc5C(F)(F)F)C4)CC3)nc21] | 689 |
| 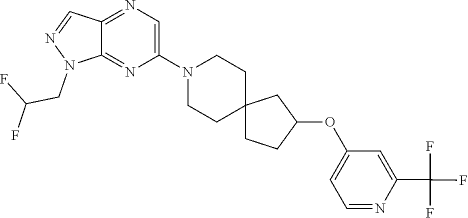 [SMILES: FC(F)Cn1ncc2ncc(N3CCC4(CCC(Oc5ccnc(C(F)(F)F)c5)C4)CC3)nc21] | 690 |
| 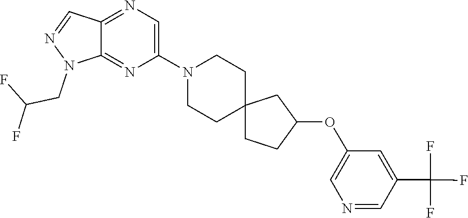 [SMILES: FC(F)Cn1ncc2ncc(N3CCC4(CCC(Oc5cncc(C(F)(F)F)c5)C4)CC3)nc21] | 691 |
| 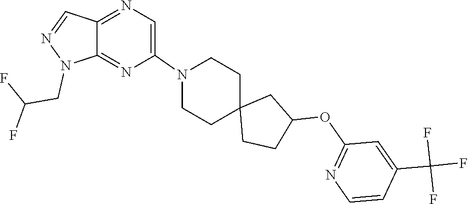 [SMILES: FC(F)Cn1ncc2ncc(N3CCC4(CCC(Oc5cc(C(F)(F)F)ccn5)C4)CC3)nc21] | 692 |
| 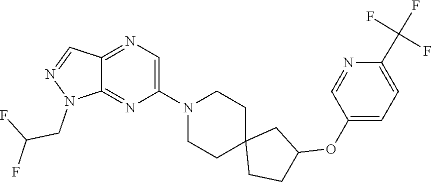 [SMILES: FC(F)Cn1ncc2ncc(N3CCC4(CCC(Oc5ccc(C(F)(F)F)nc5)C4)CC3)nc21] | 693 |
| 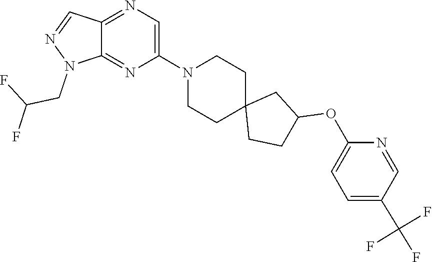 [SMILES: FC(F)Cn1ncc2ncc(N3CCC4(CCC(Oc5ccc(C(F)(F)F)cn5)C4)CC3)nc21] | 694 |
| 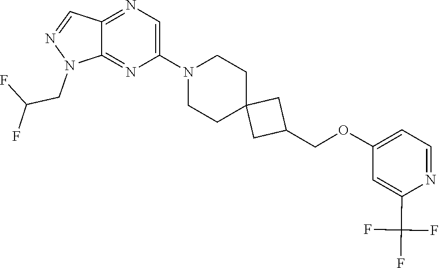 [SMILES: FC(F)Cn1ncc2ncc(N3CCC4(CC3)CC(COc3ccnc(C(F)(F)F)c3)C4)nc21] | 695 |
| 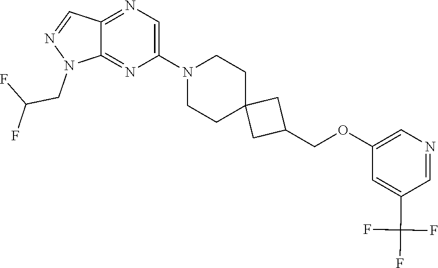 [SMILES: FC(F)Cn1ncc2ncc(N3CCC4(CC3)CC(COc3cncc(C(F)(F)F)c3)C4)nc21] | 696 |
| 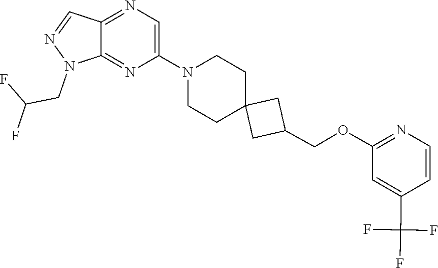 [SMILES: FC(F)Cn1ncc2ncc(N3CCC4(CC3)CC(COc3cc(C(F)(F)F)ccn3)C4)nc21] | 697 |
| 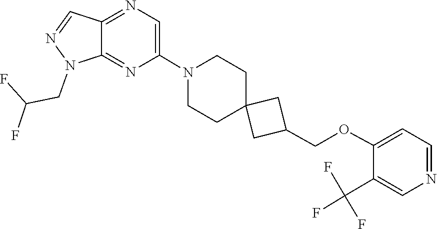 [SMILES: FC(F)Cn1ncc2ncc(N3CCC4(CC3)CC(COc3ccncc3C(F)(F)F)C4)nc21] | 698 |
| 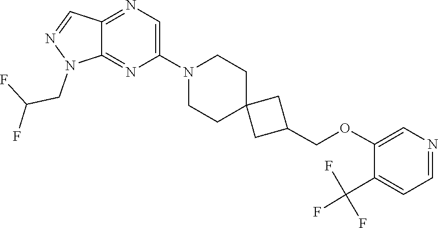 [SMILES: FC(F)Cn1ncc2ncc(N3CCC4(CC3)CC(COc3cnccc3C(F)(F)F)C4)nc21] | 699 |
| 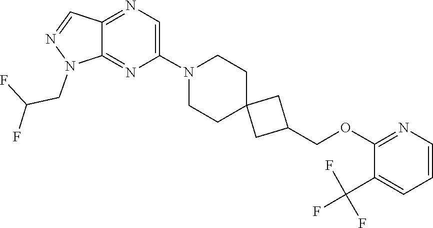 [SMILES: FC(F)Cn1ncc2ncc(N3CCC4(CC3)CC(COc3ncccc3C(F)(F)F)C4)nc21] | 700 |
| 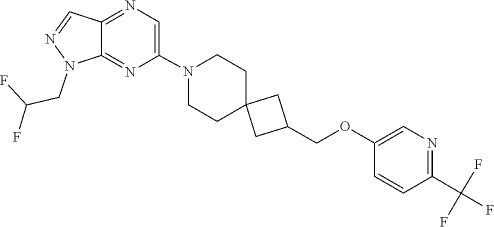 [SMILES: FC(F)Cn1ncc2ncc(N3CCC4(CC3)CC(COc3ccc(C(F)(F)F)nc3)C4)nc21] | 701 |
| 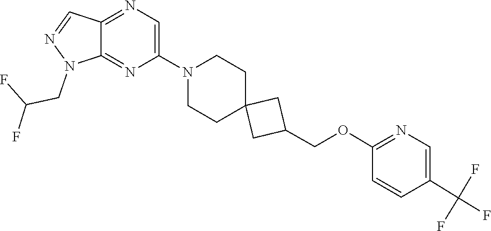 [SMILES: FC(F)Cn1ncc2ncc(N3CCC4(CC3)CC(COc3ccc(C(F)(F)F)cn3)C4)nc21] | 702 |
| 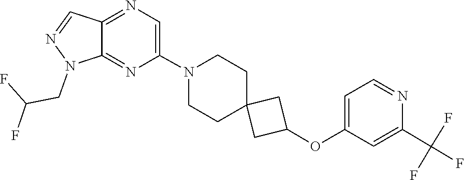 [SMILES: FC(F)Cn1ncc2ncc(N3CCC4(CC3)CC(Oc3ccnc(C(F)(F)F)c3)C4)nc21] | 703 |
| 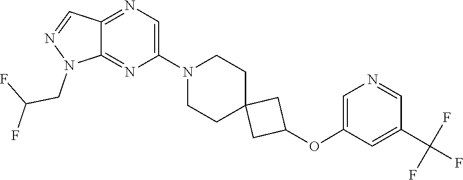 [SMILES: FC(F)Cn1ncc2ncc(N3CCC4(CC3)CC(Oc3cncc(C(F)(F)F)c3)C4)nc21] | 704 |
| 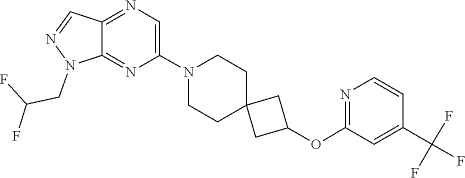 [SMILES: FC(F)Cn1ncc2ncc(N3CCC4(CC3)CC(Oc3cc(C(F)(F)F)ccn3)C4)nc21] | 705 |
| 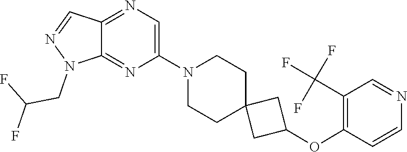 [SMILES: FC(F)Cn1ncc2ncc(N3CCC4(CC3)CC(Oc3ccncc3C(F)(F)F)C4)nc21] | 706 |
| 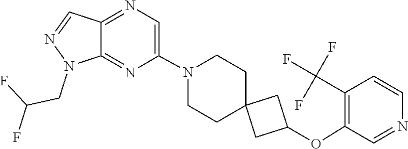 [SMILES: FC(F)Cn1ncc2ncc(N3CCC4(CC3)CC(Oc3cnccc3C(F)(F)F)C4)nc21] | 707 |
| 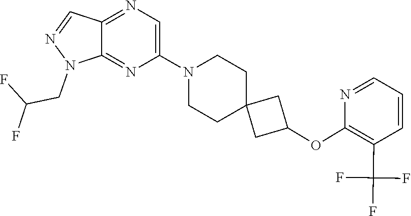 [SMILES: FC(F)Cn1ncc2ncc(N3CCC4(CC3)CC(Oc3ncccc3C(F)(F)F)C4)nc21] | 708 |
| 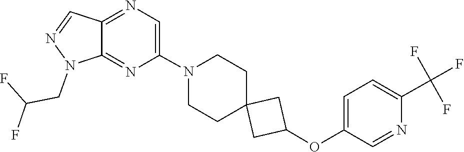 [SMILES: FC(F)Cn1ncc2ncc(N3CCC4(CC3)CC(Oc3ccc(C(F)(F)F)nc3)C4)nc21] | 709 |
| 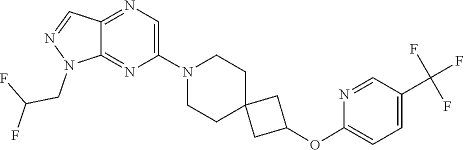 [SMILES: FC(F)Cn1ncc2ncc(N3CCC4(CC3)CC(Oc3ccc(C(F)(F)F)cn3)C4)nc21] | 710 |
| 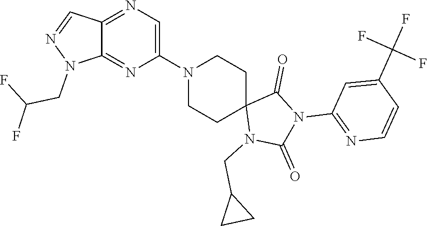 [SMILES: O=C1N(c2cc(C(F)(F)F)ccn2)C(=O)C2(CCN(c3cnc4cnn(CC(F)F)c4n3)CC2)N1CC1CC1] | 711 |
| 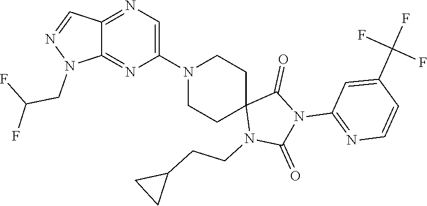 [SMILES: O=C1N(c2cc(C(F)(F)F)ccn2)C(=O)C2(CCN(c3cnc4cnn(CC(F)F)c4n3)CC2)N1CCC1CC1] | 712 |
| 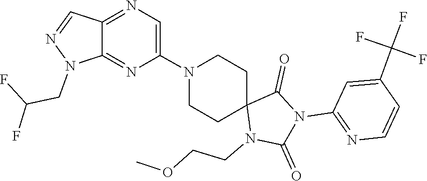 [SMILES: COCCN1C(=O)N(c2cc(C(F)(F)F)ccn2)C(=O)C12CCN(c1cnc3cnn(CC(F)F)c3n1)CC2] | 713 |
| 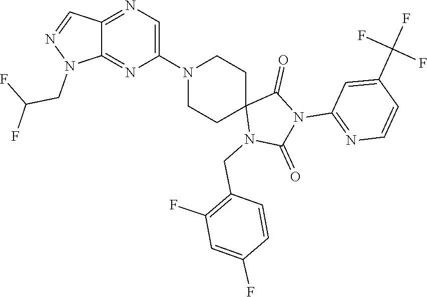 [SMILES: O=C1N(c2cc(C(F)(F)F)ccn2)C(=O)C2(CCN(c3cnc4cnn(CC(F)F)c4n3)CC2)N1Cc1ccc(F)cc1F] | 714 |
| 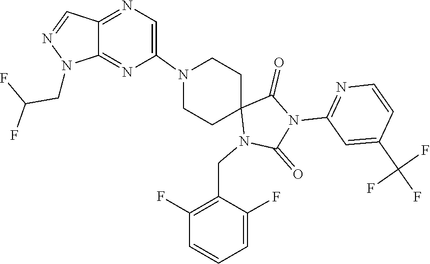 [SMILES: O=C1N(c2cc(C(F)(F)F)ccn2)C(=O)C2(CCN(c3cnc4cnn(CC(F)F)c4n3)CC2)N1Cc1c(F)cccc1F] | 715 |
| 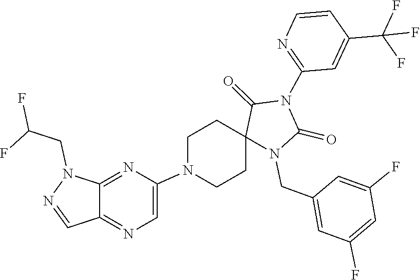 [SMILES: O=C1N(c2cc(C(F)(F)F)ccn2)C(=O)C2(CCN(c3cnc4cnn(CC(F)F)c4n3)CC2)N1Cc1cc(F)cc(F)c1] | 716 |
| 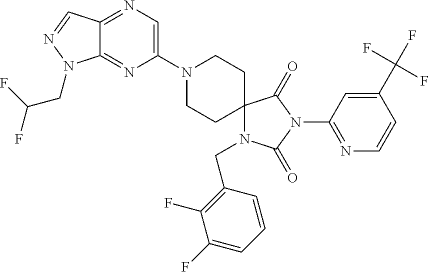 [SMILES: O=C1N(c2cc(C(F)(F)F)ccn2)C(=O)C2(CCN(c3cnc4cnn(CC(F)F)c4n3)CC2)N1Cc1cccc(F)c1F] | 717 |
| 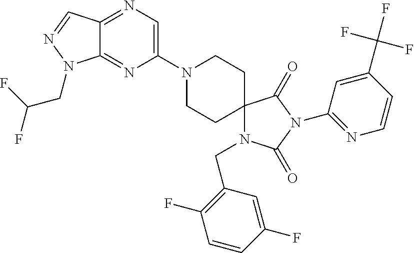 [SMILES: O=C1N(c2cc(C(F)(F)F)ccn2)C(=O)C2(CCN(c3cnc4cnn(CC(F)F)c4n3)CC2)N1Cc1cc(F)ccc1F] | 718 |
| 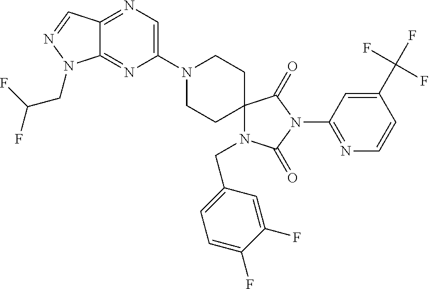 [SMILES: O=C1N(c2cc(C(F)(F)F)ccn2)C(=O)C2(CCN(c3cnc4cnn(CC(F)F)c4n3)CC2)N1Cc1ccc(F)c(F)c1] | 719 |
| 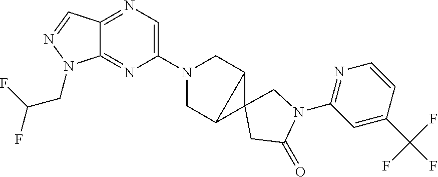 [SMILES: O=C1CC2(CN1c1cc(C(F)(F)F)ccn1)C1CN(c3cnc4cnn(CC(F)F)c4n3)CC12] | 720 |
| 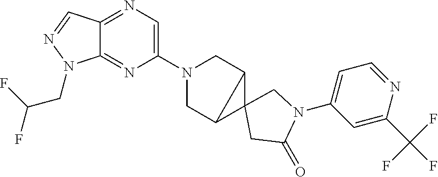 [SMILES: O=C1CC2(CN1c1ccnc(C(F)(F)F)c1)C1CN(c3cnc4cnn(CC(F)F)c4n3)CC12] | 721 |
| 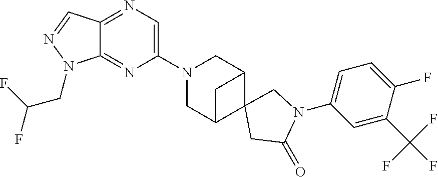 [SMILES: O=C1CC2(CN1c1ccc(F)c(C(F)(F)F)c1)C1CC2CN(c2cnc3cnn(CC(F)F)c3n2)C1] | 722 |
| 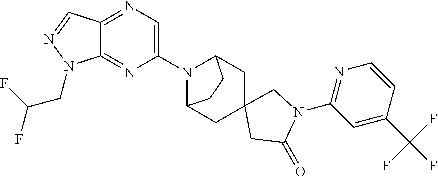 [SMILES: O=C1CC2(CC3CCC(C2)N3c2cnc3cnn(CC(F)F)c3n2)CN1c1cc(C(F)(F)F)ccn1] | 723 |
| 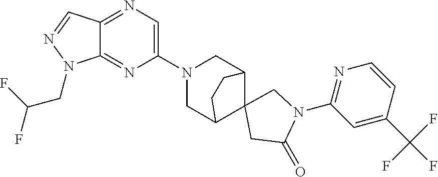 [SMILES: O=C1CC2(CN1c1cc(C(F)(F)F)ccn1)C1CCC2CN(c2cnc3cnn(CC(F)F)c3n2)C1] | 724 |
| 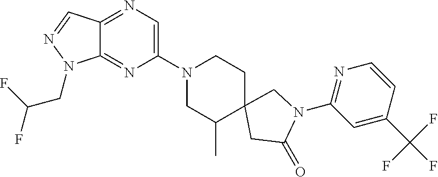 [SMILES: CC1CN(c2cnc3cnn(CC(F)F)c3n2)CCC12CC(=O)N(c1cc(C(F)(F)F)ccn1)C2] | 725 |
| 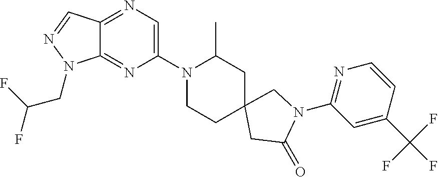 [SMILES: CC1CC2(CCN1c1cnc3cnn(CC(F)F)c3n1)CC(=O)N(c1cc(C(F)(F)F)ccn1)C2] | 726 |
| 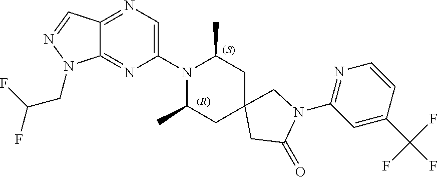 | 727 |
| 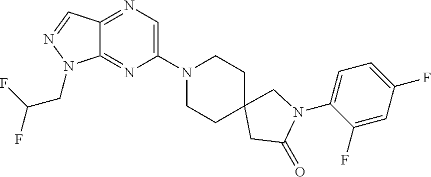 [SMILES: O=C1CC2(CCN(c3cnc4cnn(CC(F)F)c4n3)CC2)CN1c1ccc(F)cc1F] | 728 |
| 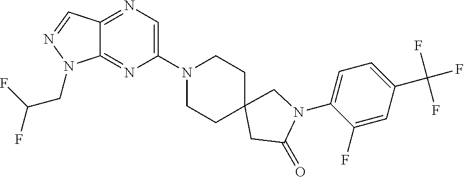 [SMILES: O=C1CC2(CCN(c3cnc4cnn(CC(F)F)c4n3)CC2)CN1c1ccc(C(F)(F)F)cc1F] | 729 |
| 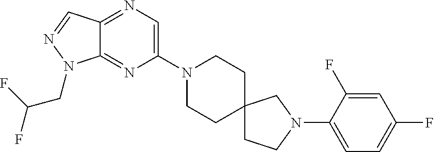 [SMILES: Fc1ccc(N2CCC3(CCN(c4cnc5cnn(CC(F)F)c5n4)CC3)C2)c(F)c1] | 730 |
| 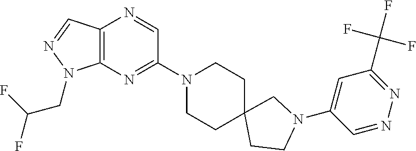 [SMILES: FC(F)Cn1ncc2ncc(N3CCC4(CCN(c5cnnc(C(F)(F)F)c5)C4)CC3)nc21] | 731 |
| 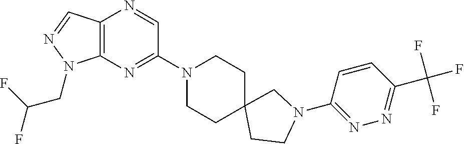 [SMILES: FC(F)Cn1ncc2ncc(N3CCC4(CCN(c5ccc(C(F)(F)F)nn5)C4)CC3)nc21] | 732 |
| 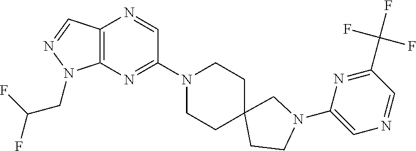 [SMILES: FC(F)Cn1ncc2ncc(N3CCC4(CCN(c5cncc(C(F)(F)F)n5)C4)CC3)nc21] | 733 |
| 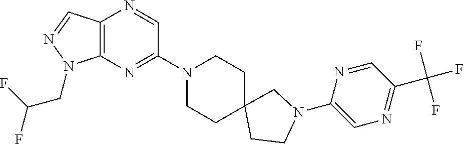 [SMILES: FC(F)Cn1ncc2ncc(N3CCC4(CCN(c5cnc(C(F)(F)F)cn5)C4)CC3)nc21] | 734 |
| 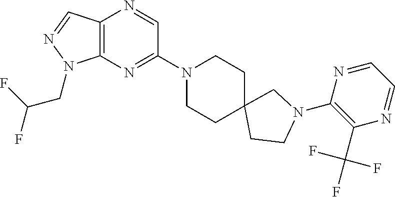 [SMILES: FC(F)Cn1ncc2ncc(N3CCC4(CC3)CCN(c3nccnc3C(F)(F)F)C4)nc21] | 735 |
| 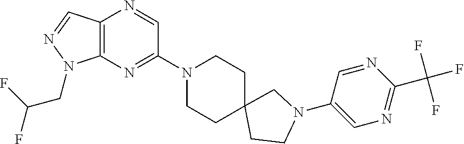 [SMILES: FC(F)Cn1ncc2ncc(N3CCC4(CCN(c5cnc(C(F)(F)F)nc5)C4)CC3)nc21] | 736 |
| 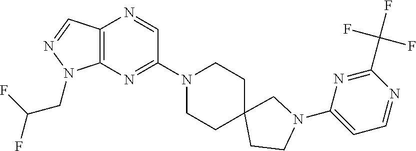 [SMILES: FC(F)Cn1ncc2ncc(N3CCC4(CCN(c5ccnc(C(F)(F)F)n5)C4)CC3)nc21] | 737 |
| 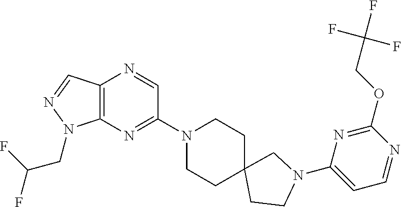 [SMILES: FC(F)Cn1ncc2ncc(N3CCC4(CCN(c5ccnc(OCC(F)(F)F)n5)C4)CC3)nc21] | 738 |
| 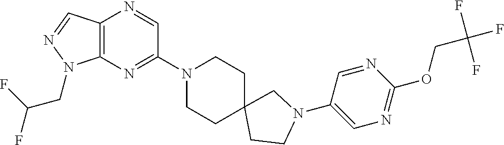 [SMILES: FC(F)Cn1ncc2ncc(N3CCC4(CCN(c5cnc(OCC(F)(F)F)nc5)C4)CC3)nc21] | 739 |
| 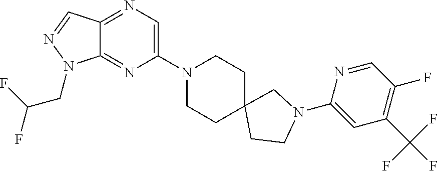 [SMILES: Fc1cnc(N2CCC3(CCN(c4cnc5cnn(CC(F)F)c5n4)CC3)C2)cc1C(F)(F)F] | 740 |
| 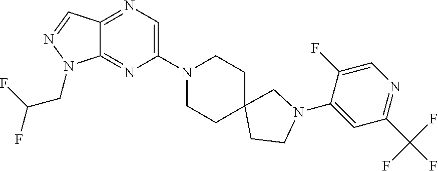 [SMILES: Fc1cnc(C(F)(F)F)cc1N1CCC2(CCN(c3cnc4cnn(CC(F)F)c4n3)CC2)C1] | 741 |
| 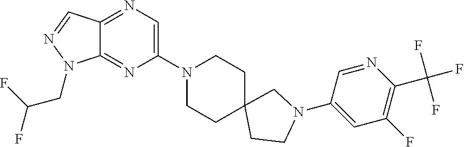 [SMILES: Fc1cc(N2CCC3(CCN(c4cnc5cnn(CC(F)F)c5n4)CC3)C2)cnc1C(F)(F)F] | 742 |
| 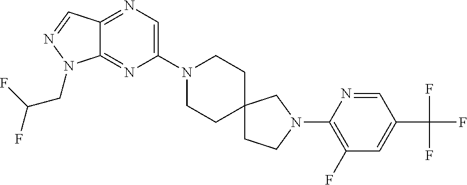 [SMILES: Fc1cc(C(F)(F)F)cnc1N1CCC2(CCN(c3cnc4cnn(CC(F)F)c4n3)CC2)C1] | 743 |
| 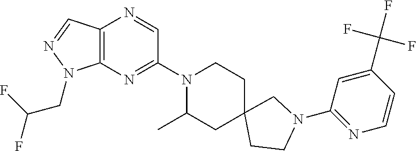 [SMILES: CC1CC2(CCN(c3cc(C(F)(F)F)ccn3)C2)CCN1c1cnc2cnn(CC(F)F)c2n1] | 745 |
| 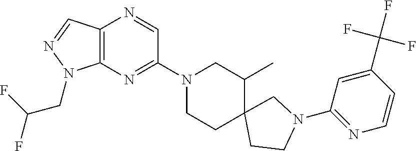 [SMILES: CC1CN(c2cnc3cnn(CC(F)F)c3n2)CCC12CCN(c1cc(C(F)(F)F)ccn1)C2] | 746 |
| 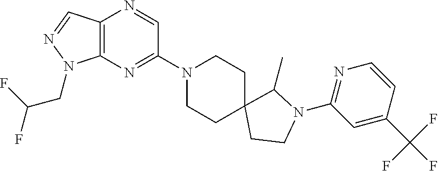 [SMILES: CC1N(c2cc(C(F)(F)F)ccn2)CCC12CCN(c1cnc3cnn(CC(F)F)c3n1)CC2] | 747 |
| 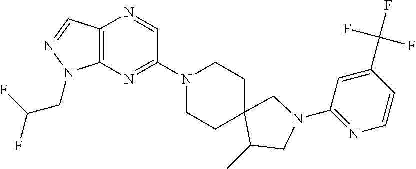 [SMILES: CC1CN(c2cc(C(F)(F)F)ccn2)CC12CCN(c1cnc3cnn(CC(F)F)c3n1)CC2] | 748 |
| 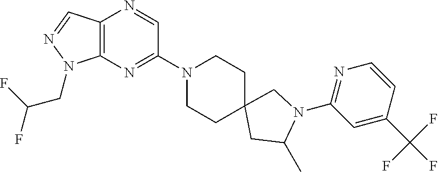 [SMILES: CC1CC2(CCN(c3cnc4cnn(CC(F)F)c4n3)CC2)CN1c1cc(C(F)(F)F)ccn1] | 749 |
| 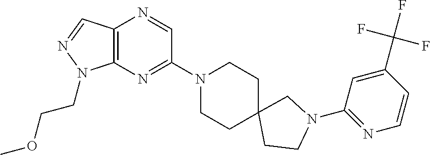 [SMILES: COCCn1ncc2ncc(N3CCC4(CCN(c5cc(C(F)(F)F)ccn5)C4)CC3)nc21] | 750 |
| 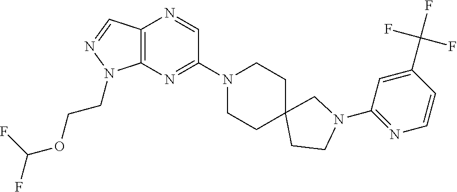 [SMILES: FC(F)OCCn1ncc2ncc(N3CCC4(CCN(c5cc(C(F)(F)F)ccn5)C4)CC3)nc21] | 751 |
| 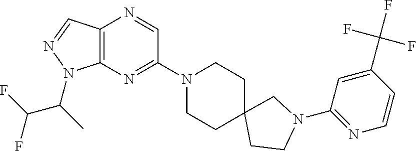 [SMILES: CC(C(F)F)n1ncc2ncc(N3CCC4(CCN(c5cc(C(F)(F)F)ccn5)C4)CC3)nc21] | 752 |
| 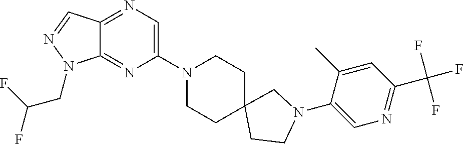 [SMILES: Cc1cc(C(F)(F)F)ncc1N1CCC2(CCN(c3cnc4cnn(CC(F)F)c4n3)CC2)C1] | 753 |
| 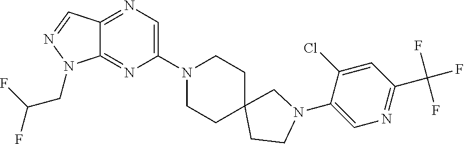 [SMILES: FC(F)Cn1ncc2ncc(N3CCC4(CC3)CCN(c3cnc(C(F)(F)F)cc3Cl)C4)nc21] | 754 |
| 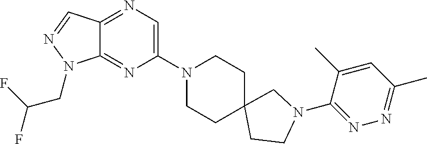 [SMILES: Cc1cc(C)c(N2CCC3(CCN(c4cnc5cnn(CC(F)F)c5n4)CC3)C2)nn1] | 755 |
| 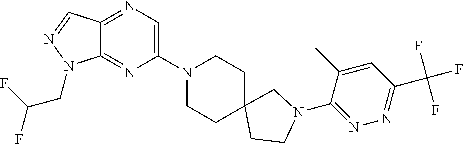 [SMILES: Cc1cc(C(F)(F)F)nnc1N1CCC2(CCN(c3cnc4cnn(CC(F)F)c4n3)CC2)C1] | 756 |
| 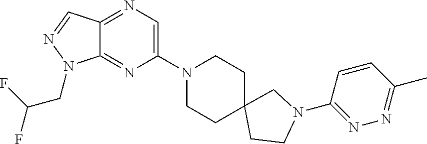 [SMILES: Cc1ccc(N2CCC3(CCN(c4cnc5cnn(CC(F)F)c5n4)CC3)C2)nn1] | 757 |
| 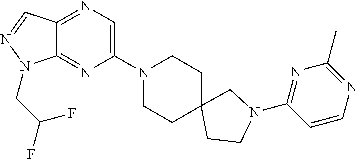 [SMILES: Cc1nccc(N2CCC3(CCN(c4cnc5cnn(CC(F)F)c5n4)CC3)C2)n1] | 758 |
| 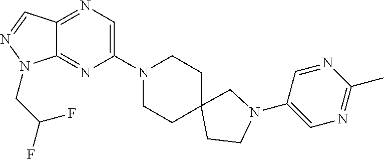 [SMILES: Cc1ncc(N2CCC3(CCN(c4cnc5cnn(CC(F)F)c5n4)CC3)C2)cn1] | 759 |
| 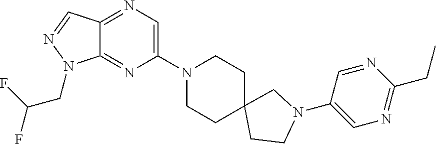 [SMILES: CCc1ncc(N2CCC3(CCN(c4cnc5cnn(CC(F)F)c5n4)CC3)C2)cn1] | 760 |
| 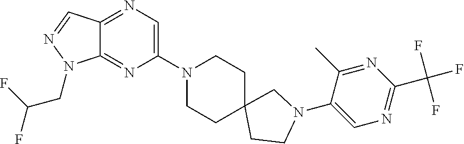 [SMILES: Cc1nc(C(F)(F)F)ncc1N1CCC2(CCN(c3cnc4cnn(CC(F)F)c4n3)CC2)C1] | 761 |
| 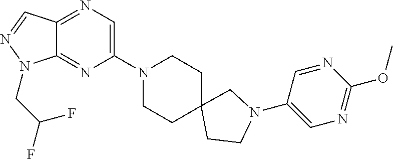 [SMILES: COc1ncc(N2CCC3(CCN(c4cnc5cnn(CC(F)F)c5n4)CC3)C2)cn1] | 762 |
| 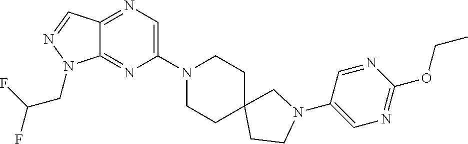 [SMILES: CCOc1ncc(N2CCC3(CCN(c4cnc5cnn(CC(F)F)c5n4)CC3)C2)cn1] | 763 |
| 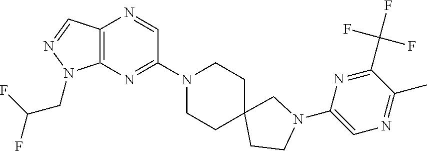 [SMILES: Cc1ncc(N2CCC3(CCN(c4cnc5cnn(CC(F)F)c5n4)CC3)C2)nc1C(F)(F)F] | 764 |
| 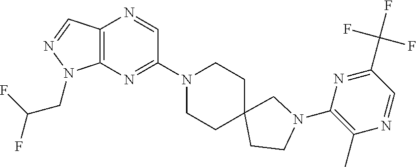 [SMILES: Cc1ncc(C(F)(F)F)nc1N1CCC2(CCN(c3cnc4cnn(CC(F)F)c4n3)CC2)C1] | 765 |
| 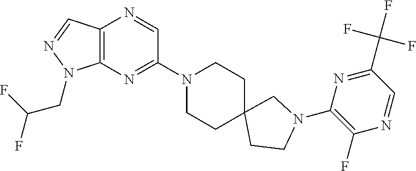 [SMILES: Fc1ncc(C(F)(F)F)nc1N1CCC2(CCN(c3cnc4cnn(CC(F)F)c4n3)CC2)C1] | 766 |
| 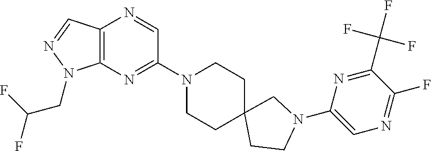 [SMILES: Fc1ncc(N2CCC3(CCN(c4cnc5cnn(CC(F)F)c5n4)CC3)C2)nc1C(F)(F)F] | 767 |
| 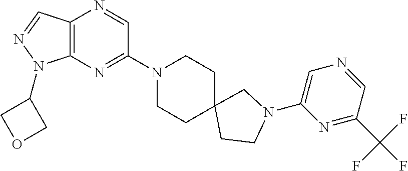 [SMILES: FC(F)(F)c1cncc(N2CCC3(CCN(c4cnc5cnn(C6COC6)c5n4)CC3)C2)n1] | 768 |
| 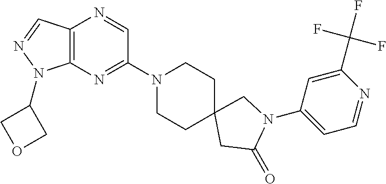 [SMILES: O=C1CC2(CCN(c3cnc4cnn(C5COC5)c4n3)CC2)CN1c1ccnc(C(F)(F)F)c1] | 769 |
| 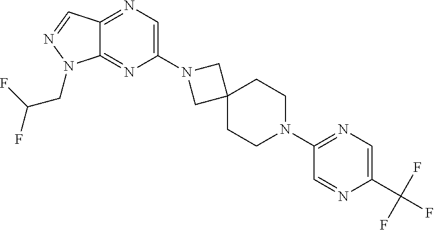 [SMILES: FC(F)Cn1ncc2ncc(N3CC4(CCN(c5cnc(C(F)(F)F)cn5)CC4)C3)nc21] | 770 |
| 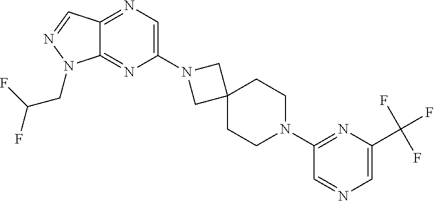 [SMILES: FC(F)Cn1ncc2ncc(N3CC4(CCN(c5cncc(C(F)(F)F)n5)CC4)C3)nc21] | 771 |
| 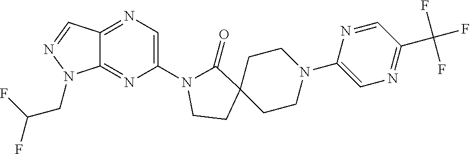 [SMILES: O=C1N(c2cnc3cnn(CC(F)F)c3n2)CCC12CCN(c1cnc(C(F)(F)F)cn1)CC2] | 772 |
| 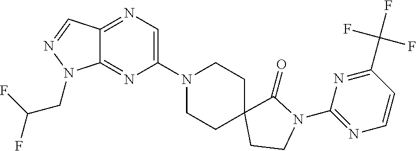 [SMILES: O=C1N(c2nccc(C(F)(F)F)n2)CCC12CCN(c1cnc3cnn(CC(F)F)c3n1)CC2] | 773 |
| 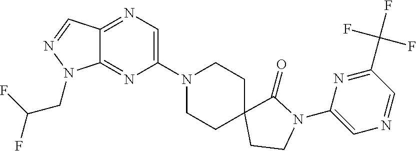 [SMILES: O=C1N(c2cncc(C(F)(F)F)n2)CCC12CCN(c1cnc3cnn(CC(F)F)c3n1)CC2] | 774 |
| 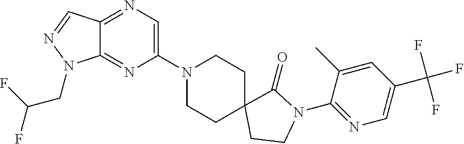 [SMILES: Cc1cc(C(F)(F)F)cnc1N1CCC2(CCN(c3cnc4cnn(CC(F)F)c4n3)CC2)C1=O] | 775 |
| 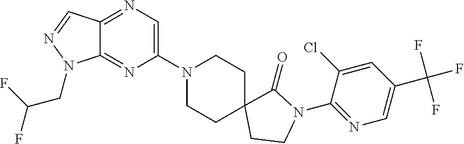 [SMILES: O=C1N(c2ncc(C(F)(F)F)cc2Cl)CCC12CCN(c1cnc3cnn(CC(F)F)c3n1)CC2] | 776 |
| 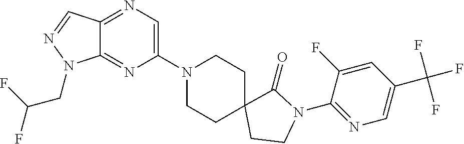 [SMILES: O=C1N(c2ncc(C(F)(F)F)cc2F)CCC12CCN(c1cnc3cnn(CC(F)F)c3n1)CC2] | 777 |
| 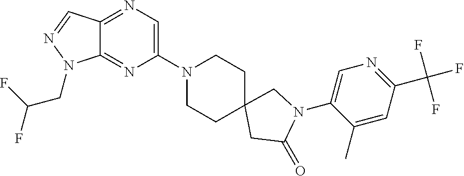 [SMILES: Cc1cc(C(F)(F)F)ncc1N1CC2(CCN(c3cnc4cnn(CC(F)F)c4n3)CC2)CC1=O] | 778 |
| 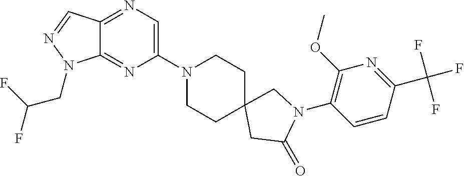 [SMILES: COc1nc(C(F)(F)F)ccc1N1CC2(CCN(c3cnc4cnn(CC(F)F)c4n3)CC2)CC1=O] | 779 |
| 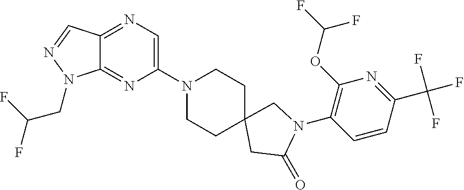 [SMILES: O=C1CC2(CCN(c3cnc4cnn(CC(F)F)c4n3)CC2)CN1c1ccc(C(F)(F)F)nc1OC(F)F] | 780 |
| 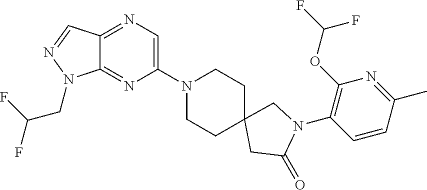 [SMILES: Cc1ccc(N2CC3(CCN(c4cnc5cnn(CC(F)F)c5n4)CC3)CC2=O)c(OC(F)F)n1] | 781 |
| 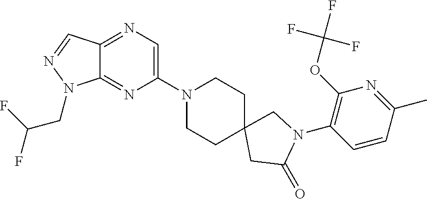 [SMILES: Cc1ccc(N2CC3(CCN(c4cnc5cnn(CC(F)F)c5n4)CC3)CC2=O)c(OC(F)(F)F)n1] | 782 |
| 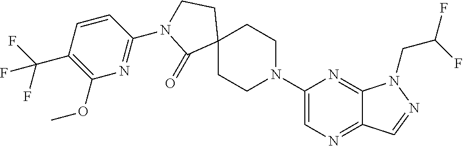 [SMILES: COc1nc(N2CCC3(CCN(c4cnc5cnn(CC(F)F)c5n4)CC3)C2=O)ccc1C(F)(F)F] | 783 |
| 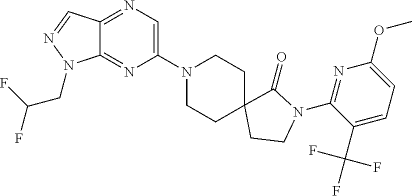 [SMILES: COc1ccc(C(F)(F)F)c(N2CCC3(CCN(c4cnc5cnn(CC(F)F)c5n4)CC3)C2=O)n1] | 784 |
| 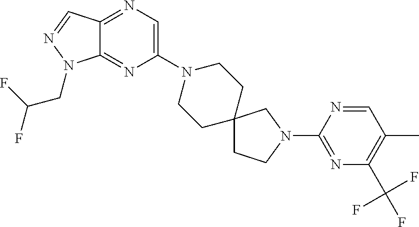 [SMILES: Cc1cnc(N2CCC3(CCN(c4cnc5cnn(CC(F)F)c5n4)CC3)C2)nc1C(F)(F)F] | 785 |
| 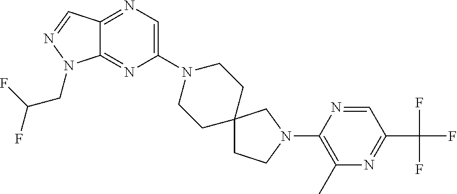 [SMILES: Cc1nc(C(F)(F)F)cnc1N1CCC2(CCN(c3cnc4cnn(CC(F)F)c4n3)CC2)C1] | 786 |
| 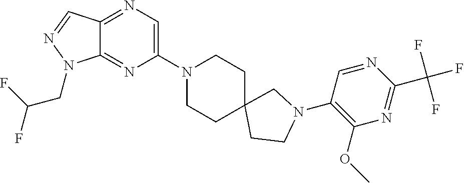 [SMILES: COc1nc(C(F)(F)F)ncc1N1CCC2(CCN(c3cnc4cnn(CC(F)F)c4n3)CC2)C1] | 787 |
| 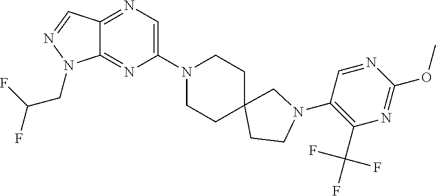 [SMILES: COc1ncc(N2CCC3(CCN(c4cnc5cnn(CC(F)F)c5n4)CC3)C2)c(C(F)(F)F)n1] | 788 |
| 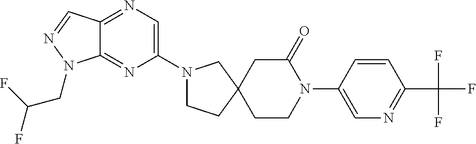 [SMILES: O=C1CC2(CCN(c3cnc4cnn(CC(F)F)c4n3)C2)CCN1c1ccc(C(F)(F)F)nc1] | 789 |
| 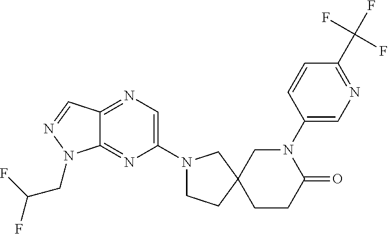 [SMILES: O=C1CCC2(CCN(c3cnc4cnn(CC(F)F)c4n3)C2)CN1c1ccc(C(F)(F)F)nc1] | 790 |
| 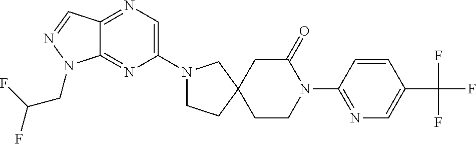 [SMILES: O=C1CC2(CCN(c3cnc4cnn(CC(F)F)c4n3)C2)CCN1c1ccc(C(F)(F)F)cn1] | 791 |
| 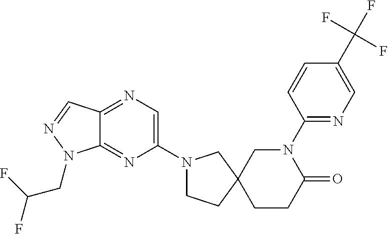 [SMILES: O=C1CCC2(CCN(c3cnc4cnn(CC(F)F)c4n3)C2)CN1c1ccc(C(F)(F)F)cn1] | 792 |
| 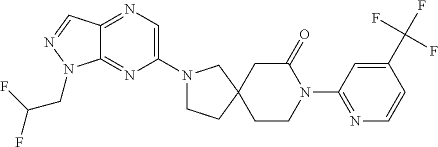 [SMILES: O=C1CC2(CCN(c3cnc4cnn(CC(F)F)c4n3)C2)CCN1c1cc(C(F)(F)F)ccn1] | 793 |
| 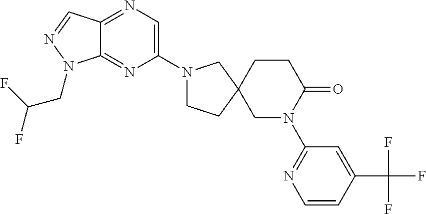 [SMILES: O=C1CCC2(CCN(c3cnc4cnn(CC(F)F)c4n3)C2)CN1c1cc(C(F)(F)F)ccn1] | 794 |
| 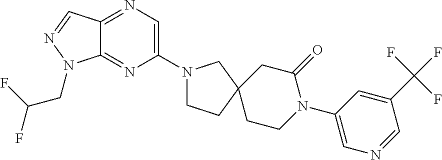 [SMILES: O=C1CC2(CCN(c3cnc4cnn(CC(F)F)c4n3)C2)CCN1c1cncc(C(F)(F)F)c1] | 795 |
| 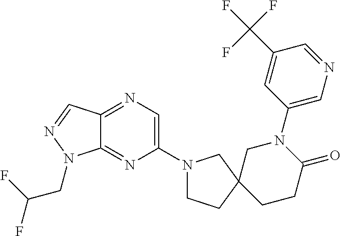 [SMILES: O=C1CCC2(CCN(c3cnc4cnn(CC(F)F)c4n3)C2)CN1c1cncc(C(F)(F)F)c1] | 796 |
| 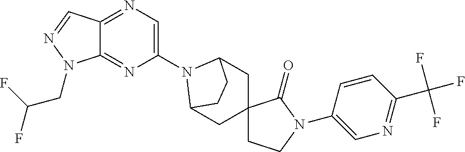 [SMILES: O=C1N(c2ccc(C(F)(F)F)nc2)CCC12CC1CCC(C2)N1c1cnc2cnn(CC(F)F)c2n1] | 797 |
| 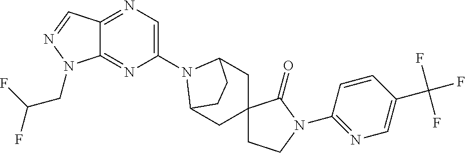 [SMILES: O=C1N(c2ccc(C(F)(F)F)cn2)CCC12CC1CCC(C2)N1c1cnc2cnn(CC(F)F)c2n1] | 798 |
| 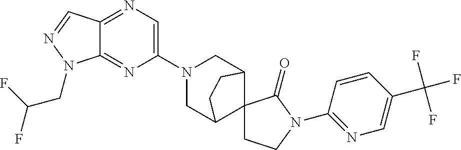 [SMILES: O=C1N(c2ccc(C(F)(F)F)cn2)CCC12C1CCC2CN(c2cnc3cnn(CC(F)F)c3n2)C1] | 799 |
| 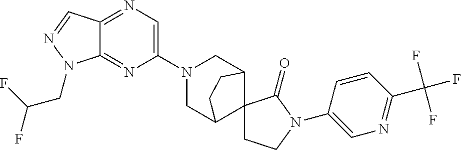 [SMILES: O=C1N(c2ccc(C(F)(F)F)nc2)CCC12C1CCC2CN(c2cnc3cnn(CC(F)F)c3n2)C1] | 800 |
| 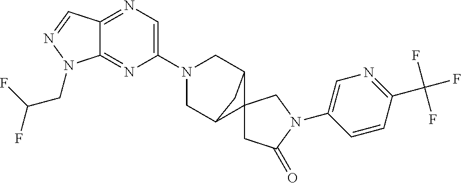 [SMILES: O=C1CC2(CN1c1ccc(C(F)(F)F)nc1)C1CC2CN(c2cnc3cnn(CC(F)F)c3n2)C1] | 801 |
| 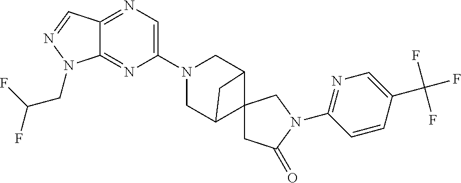 [SMILES: O=C1CC2(CN1c1ccc(C(F)(F)F)cn1)C1CC2CN(c2cnc3cnn(CC(F)F)c3n2)C1] | 802 |
| 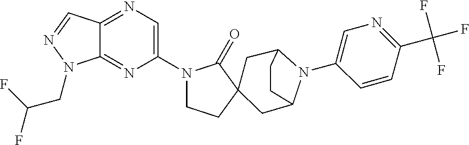 [SMILES: O=C1N(c2cnc3cnn(CC(F)F)c3n2)CCC12CC1CCC(C2)N1c1ccc(C(F)(F)F)nc1] | 803 |
| 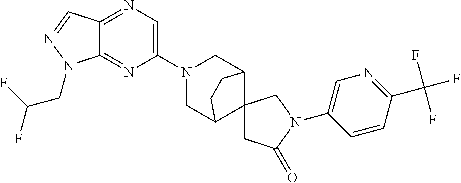 [SMILES: O=C1CC2(CN1c1ccc(C(F)(F)F)nc1)C1CCC2CN(c2cnc3cnn(CC(F)F)c3n2)C1] | 804 |
| 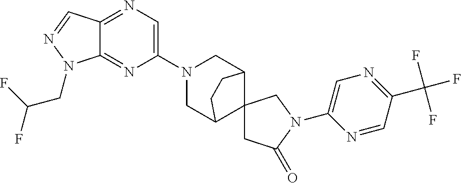 [SMILES: O=C1CC2(CN1c1cnc(C(F)(F)F)cn1)C1CCC2CN(c2cnc3cnn(CC(F)F)c3n2)C1] | 805 |
| 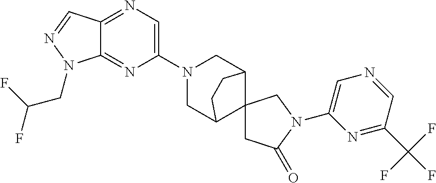 [SMILES: O=C1CC2(CN1c1cncc(C(F)(F)F)n1)C1CCC2CN(c2cnc3cnn(CC(F)F)c3n2)C1] | 806 |
| 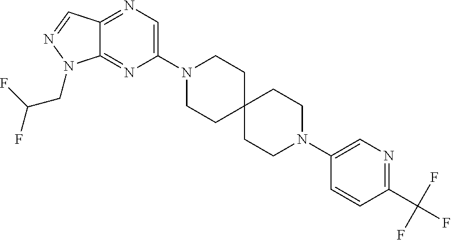 [SMILES: FC(F)Cn1ncc2ncc(N3CCC4(CCN(c5ccc(C(F)(F)F)nc5)CC4)CC3)nc21] | 807 |
| 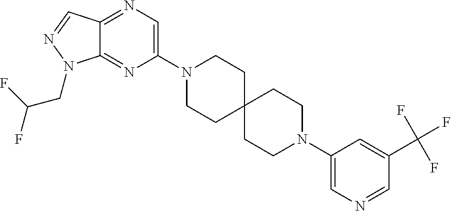 [SMILES: FC(F)Cn1ncc2ncc(N3CCC4(CCN(c5cncc(C(F)(F)F)c5)CC4)CC3)nc21] | 808 |
| 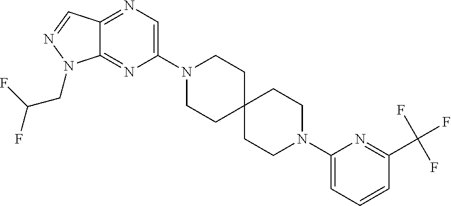 [SMILES: FC(F)Cn1ncc2ncc(N3CCC4(CCN(c5cccc(C(F)(F)F)n5)CC4)CC3)nc21] | 809 |
| 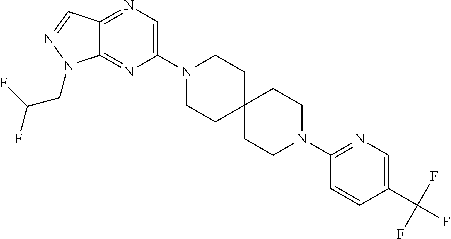 [SMILES: FC(F)Cn1ncc2ncc(N3CCC4(CCN(c5ccc(C(F)(F)F)cn5)CC4)CC3)nc21] | 810 |
| 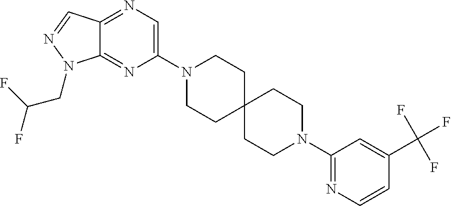 [SMILES: FC(F)Cn1ncc2ncc(N3CCC4(CCN(c5cc(C(F)(F)F)ccn5)CC4)CC3)nc21] | 811 |
| 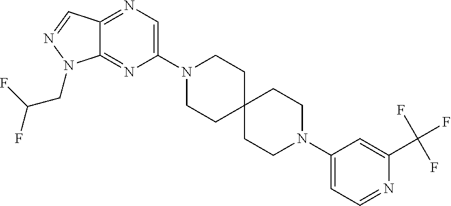 [SMILES: FC(F)Cn1ncc2ncc(N3CCC4(CCN(c5ccnc(C(F)(F)F)c5)CC4)CC3)nc21] | 812 |
| 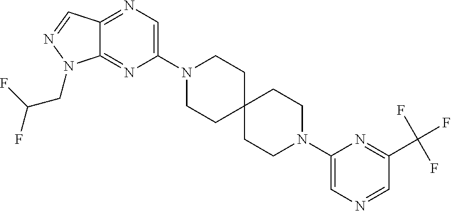 [SMILES: FC(F)Cn1ncc2ncc(N3CCC4(CCN(c5cncc(C(F)(F)F)n5)CC4)CC3)nc21] | 813 |
| 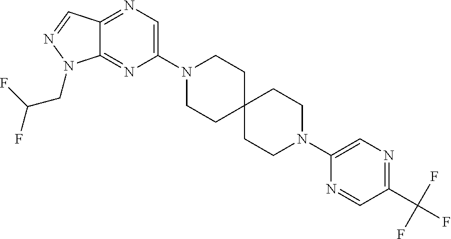 [SMILES: FC(F)Cn1ncc2ncc(N3CCC4(CCN(c5cnc(C(F)(F)F)cn5)CC4)CC3)nc21] | 814 |
| 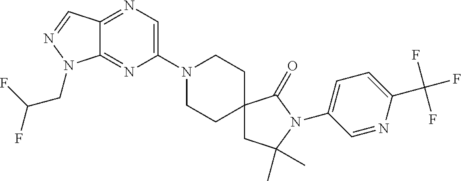 [SMILES: CC1(C)CC2(CCN(c3cnc4cnn(CC(F)F)c4n3)CC2)C(=O)N1c1ccc(C(F)(F)F)nc1] | 815 |
| 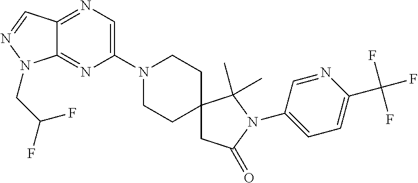 [SMILES: CC1(C)N(c2ccc(C(F)(F)F)nc2)C(=O)CC12CCN(c1cnc3cnn(CC(F)F)c3n1)CC2] | 816 |
| 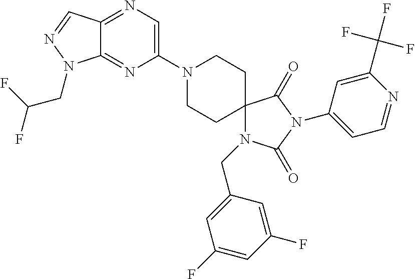 [SMILES: O=C1N(c2ccnc(C(F)(F)F)c2)C(=O)C2(CCN(c3cnc4cnn(CC(F)F)c4n3)CC2)N1Cc1cc(F)cc(F)c1] | 817 |
| 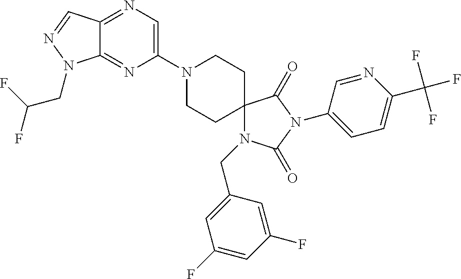 [SMILES: O=C1N(c2ccc(C(F)(F)F)nc2)C(=O)C2(CCN(c3cnc4cnn(CC(F)F)c4n3)CC2)N1Cc1cc(F)cc(F)c1] | 818 |
| 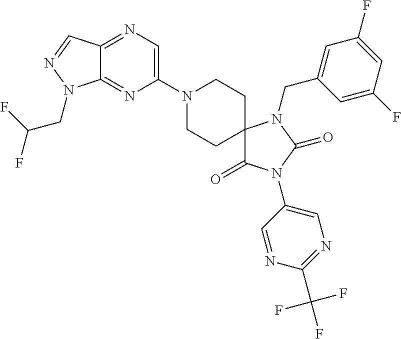 [SMILES: O=C1N(c2cnc(C(F)(F)F)nc2)C(=O)C2(CCN(c3cnc4cnn(CC(F)F)c4n3)CC2)N1Cc1cc(F)cc(F)c1] | 819 |
| 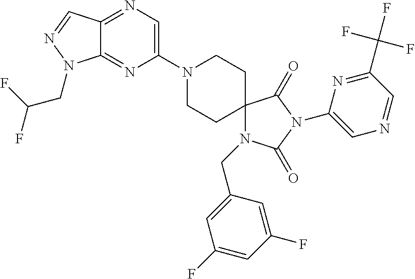 [SMILES: O=C1N(c2cncc(C(F)(F)F)n2)C(=O)C2(CCN(c3cnc4cnn(CC(F)F)c4n3)CC2)N1Cc1cc(F)cc(F)c1] | 820 |
| 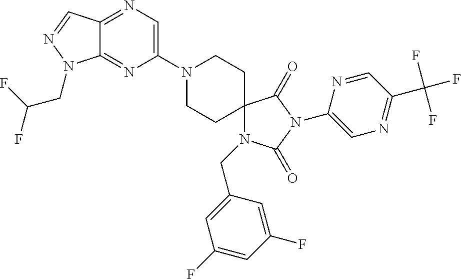 [SMILES: O=C1N(c2cnc(C(F)(F)F)cn2)C(=O)C2(CCN(c3cnc4cnn(CC(F)F)c4n3)CC2)N1Cc1cc(F)cc(F)c1] | 821 |
| 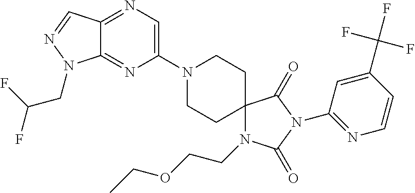 [SMILES: CCOCCN1C(=O)N(c2cc(C(F)(F)F)ccn2)C(=O)C12CCN(c1cnc3cnn(CC(F)F)c3n1)CC2] | 822 |
| 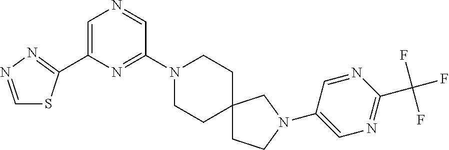 [SMILES: FC(F)(F)c1ncc(N2CCC3(CCN(c4cncc(-c5nncs5)n4)CC3)C2)cn1] | 823 |
| 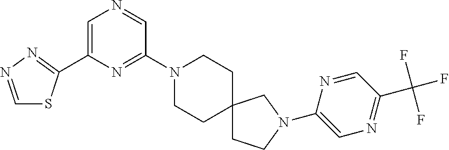 [SMILES: FC(F)(F)c1cnc(N2CCC3(CCN(c4cncc(-c5nncs5)n4)CC3)C2)cn1] | 824 |
| 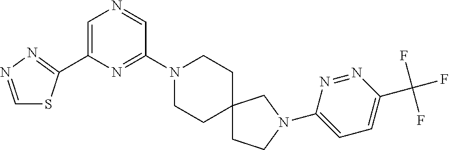 [SMILES: FC(F)(F)c1ccc(N2CCC3(CCN(c4cncc(-c5nncs5)n4)CC3)C2)nn1] | 825 |
| 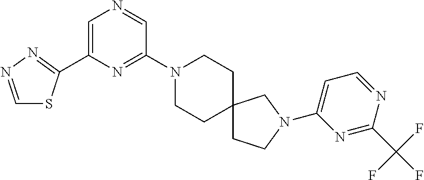 [SMILES: FC(F)(F)c1nccc(N2CCC3(CCN(c4cncc(-c5nncs5)n4)CC3)C2)n1] | 826 |
| 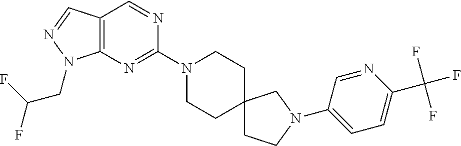 [SMILES: FC(F)Cn1ncc2cnc(N3CCC4(CCN(c5ccc(C(F)(F)F)nc5)C4)CC3)nc21] | 827 |
| 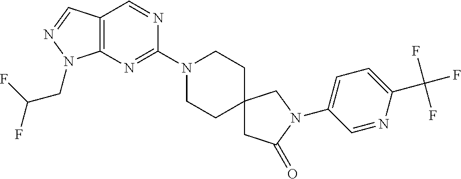 [SMILES: O=C1CC2(CCN(c3ncc4cnn(CC(F)F)c4n3)CC2)CN1c1ccc(C(F)(F)F)nc1] | 828 |
| 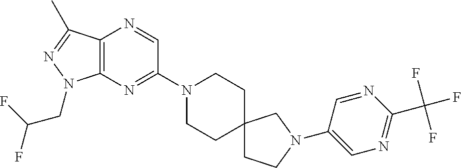 [SMILES: Cc1nn(CC(F)F)c2nc(N3CCC4(CCN(c5cnc(C(F)(F)F)nc5)C4)CC3)cnc12] | 829 |
| 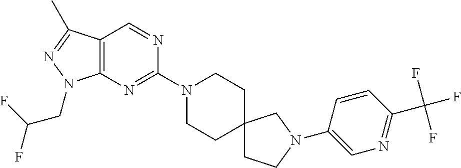 [SMILES: Cc1nn(CC(F)F)c2nc(N3CCC4(CCN(c5ccc(C(F)(F)F)nc5)C4)CC3)ncc12] | 830 |
| 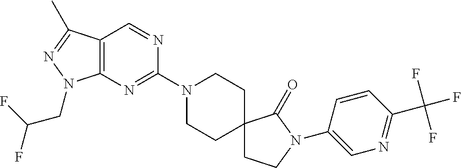 [SMILES: Cc1nn(CC(F)F)c2nc(N3CCC4(CC3)CCN(c3ccc(C(F)(F)F)nc3)C4=O)ncc12] | 831 |
| 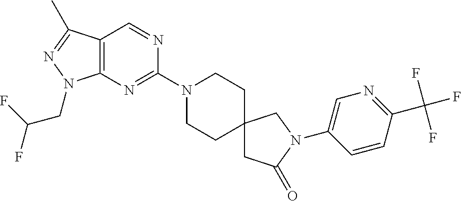 [SMILES: Cc1nn(CC(F)F)c2nc(N3CCC4(CC3)CC(=O)N(c3ccc(C(F)(F)F)nc3)C4)ncc12] | 832 |
| 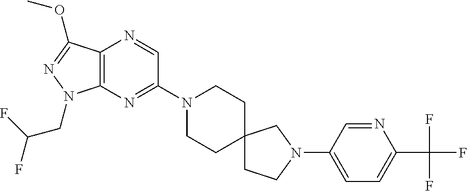 [SMILES: COc1nn(CC(F)F)c2nc(N3CCC4(CCN(c5ccc(C(F)(F)F)nc5)C4)CC3)cnc12] | 833 |
| 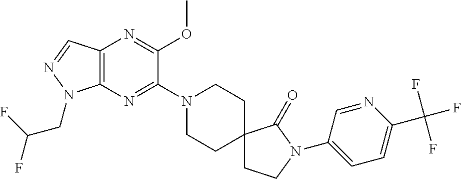 [SMILES: COc1nc2cnn(CC(F)F)c2nc1N1CCC2(CC1)CCN(c1ccc(C(F)(F)F)nc1)C2=O] | 834 |
| 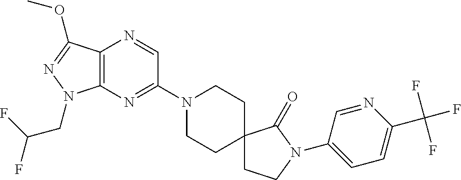 [SMILES: COc1nn(CC(F)F)c2nc(N3CCC4(CC3)CCN(c3ccc(C(F)(F)F)nc3)C4=O)cnc12] | 835 |
| 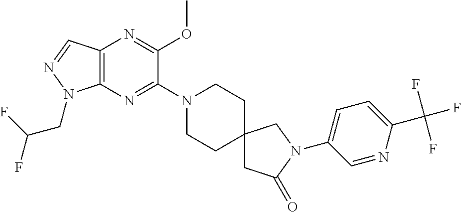 [SMILES: COc1nc2cnn(CC(F)F)c2nc1N1CCC2(CC1)CC(=O)N(c1ccc(C(F)(F)F)nc1)C2] | 836 |
| 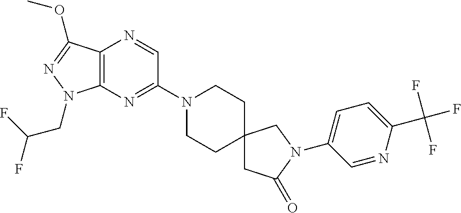 [SMILES: COc1nn(CC(F)F)c2nc(N3CCC4(CC3)CC(=O)N(c3ccc(C(F)(F)F)nc3)C4)cnc12] | 837 |
| 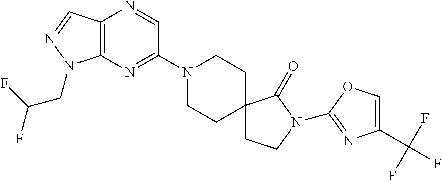 [SMILES: O=C1N(c2nc(C(F)(F)F)co2)CCC12CCN(c1cnc3cnn(CC(F)F)c3n1)CC2] | 838 |
| 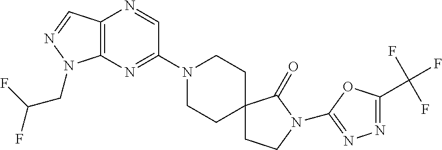 [SMILES: O=C1N(c2nnc(C(F)(F)F)o2)CCC12CCN(c1cnc3cnn(CC(F)F)c3n1)CC2] | 839 |
| 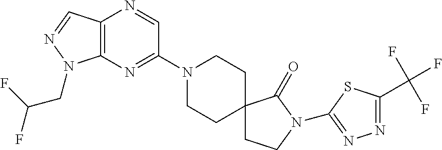 [SMILES: O=C1N(c2nnc(C(F)(F)F)s2)CCC12CCN(c1cnc3cnn(CC(F)F)c3n1)CC2] | 840 |
| 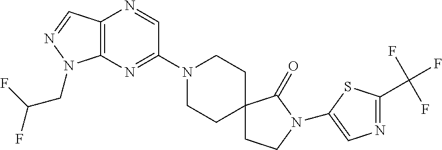 [SMILES: O=C1N(c2cnc(C(F)(F)F)s2)CCC12CCN(c1cnc3cnn(CC(F)F)c3n1)CC2] | 841 |
| 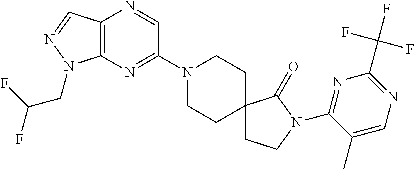 [SMILES: Cc1cnc(C(F)(F)F)nc1N1CCC2(CCN(c3cnc4cnn(CC(F)F)c4n3)CC2)C1=O] | 842 |
| 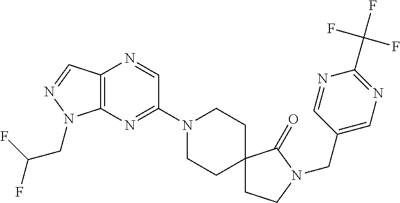 [SMILES: O=C1N(Cc2cnc(C(F)(F)F)nc2)CCC12CCN(c1cnc3cnn(CC(F)F)c3n1)CC2] | 843 |
| 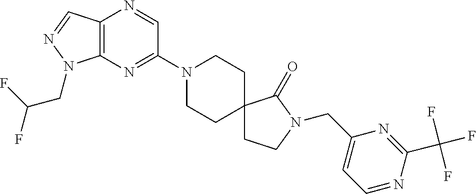 [SMILES: O=C1N(Cc2ccnc(C(F)(F)F)n2)CCC12CCN(c1cnc3cnn(CC(F)F)c3n1)CC2] | 844 |
| 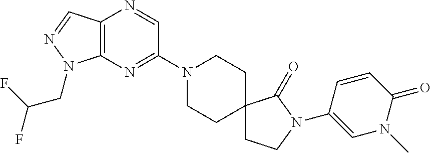 [SMILES: Cn1cc(N2CCC3(CCN(c4cnc5cnn(CC(F)F)c5n4)CC3)C2=O)ccc1=O] | 845 |
| 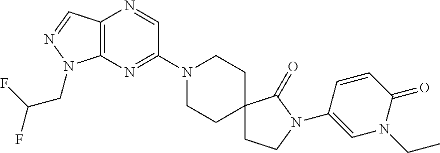 [SMILES: CCn1cc(N2CCC3(CCN(c4cnc5cnn(CC(F)F)c5n4)CC3)C2=O)ccc1=O] | 846 |
| 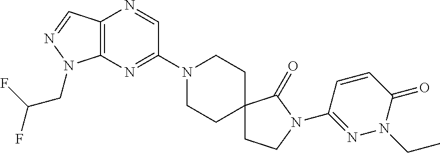 [SMILES: CCn1nc(N2CCC3(CCN(c4cnc5cnn(CC(F)F)c5n4)CC3)C2=O)ccc1=O] | 847 |
| 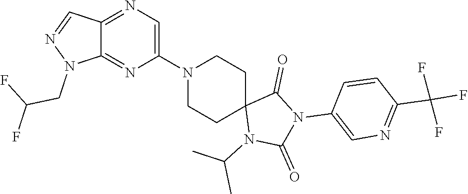 [SMILES: CC(C)N1C(=O)N(c2ccc(C(F)(F)F)nc2)C(=O)C12CCN(c1cnc3cnn(CC(F)F)c3n1)CC2] | 848 |
| 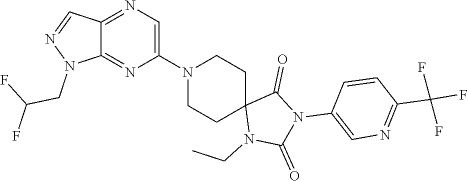 [SMILES: CCN1C(=O)N(c2ccc(C(F)(F)F)nc2)C(=O)C12CCN(c1cnc3cnn(CC(F)F)c3n1)CC2] | 849 |
| 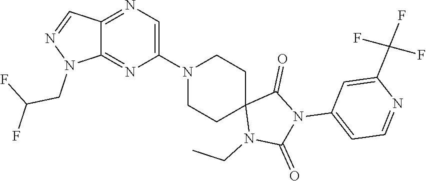 [SMILES: CCN1C(=O)N(c2ccnc(C(F)(F)F)c2)C(=O)C12CCN(c1cnc3cnn(CC(F)F)c3n1)CC2] | 850 |
| 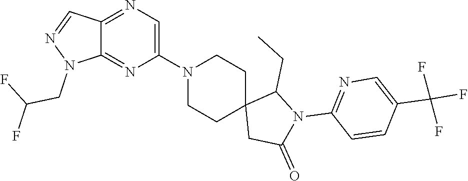 [SMILES: CCC1N(c2ccc(C(F)(F)F)cn2)C(=O)CC12CCN(c1cnc3cnn(CC(F)F)c3n1)CC2] | 851 |
| 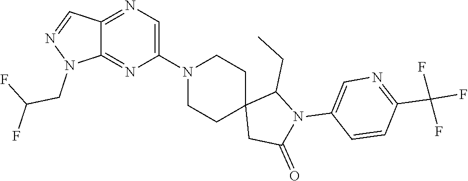 [SMILES: CCC1N(c2ccc(C(F)(F)F)nc2)C(=O)CC12CCN(c1cnc3cnn(CC(F)F)c3n1)CC2] | 852 |
| 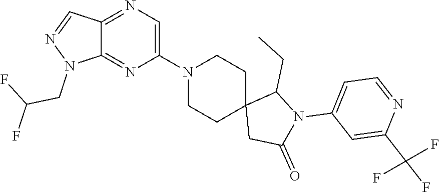 [SMILES: CCC1N(c2ccnc(C(F)(F)F)c2)C(=O)CC12CCN(c1cnc3cnn(CC(F)F)c3n1)CC2] | 853 |
| 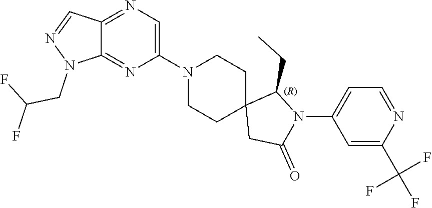 | 854 |
| 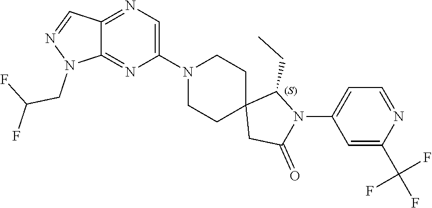 | 855 |
| 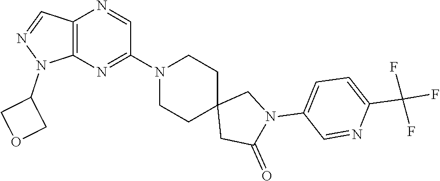 [SMILES: O=C1CC2(CCN(c3cnc4cnn(C5COC5)c4n3)CC2)CN1c1ccc(C(F)(F)F)nc1] | 856 |
| 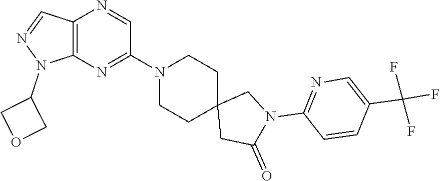 [SMILES: O=C1CC2(CCN(c3cnc4cnn(C5COC5)c4n3)CC2)CN1c1ccc(C(F)(F)F)cn1] | 857 |
| 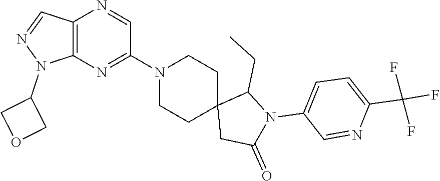 [SMILES: CCC1N(c2ccc(C(F)(F)F)nc2)C(=O)CC12CCN(c1cnc3cnn(C4COC4)c3n1)CC2] | 858 |
| 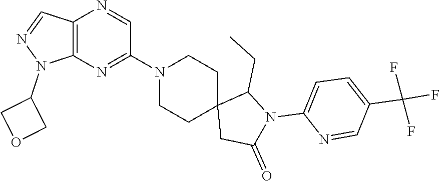 [SMILES: CCC1N(c2ccc(C(F)(F)F)cn2)C(=O)CC12CCN(c1cnc3cnn(C4COC4)c3n1)CC2] | 859 |
| 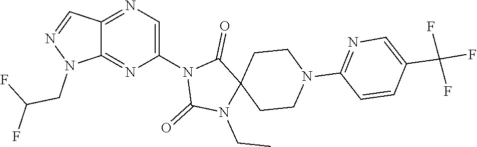 [SMILES: CCN1C(=O)N(c2cnc3cnn(CC(F)F)c3n2)C(=O)C12CCN(c1ccc(C(F)(F)F)cn1)CC2] | 860 |
| 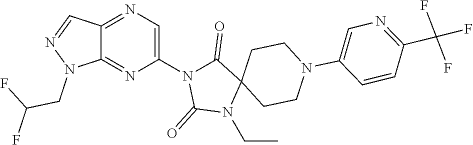 [SMILES: CCN1C(=O)N(c2cnc3cnn(CC(F)F)c3n2)C(=O)C12CCN(c1ccc(C(F)(F)F)nc1)CC2] | 861 |
| 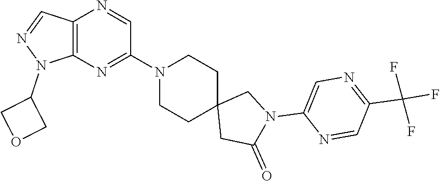 [SMILES: O=C1CC2(CCN(c3cnc4cnn(C5COC5)c4n3)CC2)CN1c1cnc(C(F)(F)F)cn1] | 862 |
| 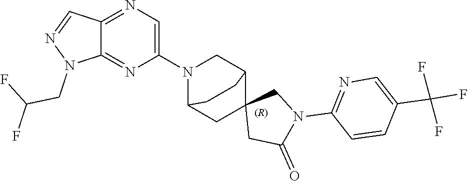 | 863 |
| 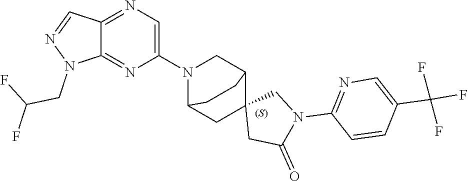 | 864 |
| 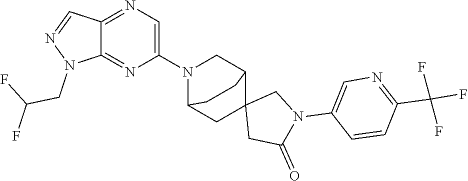 [SMILES: O=C1CC2(CC3CCC2CN3c2cnc3cnn(CC(F)F)c3n2)CN1c1ccc(C(F)(F)F)nc1] | 865 |
| 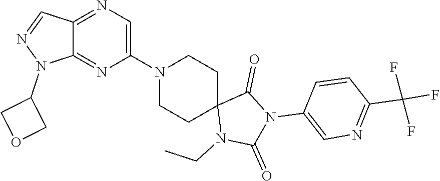 [SMILES: CCN1C(=O)N(c2ccc(C(F)(F)F)nc2)C(=O)C12CCN(c1cnc3cnn(C4COC4)c3n1)CC2] | 866 |
| 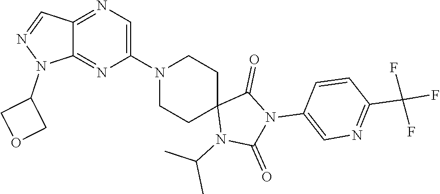 [SMILES: CC(C)N1C(=O)N(c2ccc(C(F)(F)F)nc2)C(=O)C12CCN(c1cnc3cnn(C4COC4)c3n1)CC2] | 867 |
| 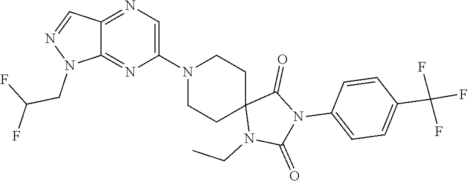 [SMILES: CCN1C(=O)N(c2ccc(C(F)(F)F)cc2)C(=O)C12CCN(c1cnc3cnn(CC(F)F)c3n1)CC2] | 868 |
| 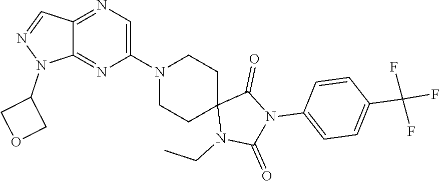 [SMILES: CCN1C(=O)N(c2ccc(C(F)(F)F)cc2)C(=O)C12CCN(c1cnc3cnn(C4COC4)c3n1)CC2] | 869 |
| 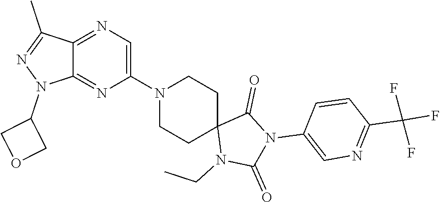 [SMILES: CCN1C(=O)N(c2ccc(C(F)(F)F)nc2)C(=O)C12CCN(c1cnc3c(C)nn(C4COC4)c3n1)CC2] | 870 |
| 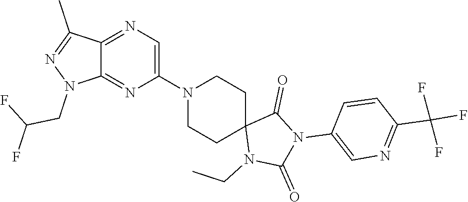 [SMILES: CCN1C(=O)N(c2ccc(C(F)(F)F)nc2)C(=O)C12CCN(c1cnc3c(C)nn(CC(F)F)c3n1)CC2] | 871 |
| 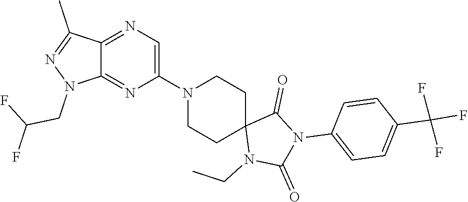 [SMILES: CCN1C(=O)N(c2ccc(C(F)(F)F)cc2)C(=O)C12CCN(c1cnc3c(C)nn(CC(F)F)c3n1)CC2] | 872 |
| 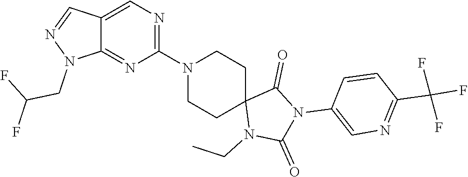 [SMILES: CCN1C(=O)N(c2ccc(C(F)(F)F)nc2)C(=O)C12CCN(c1ncc3cnn(CC(F)F)c3n1)CC2] | 873 |
| 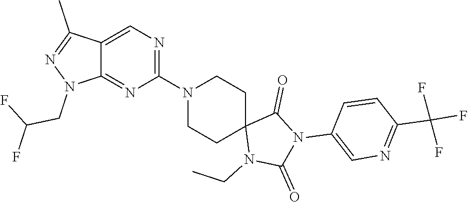 [SMILES: CCN1C(=O)N(c2ccc(C(F)(F)F)nc2)C(=O)C12CCN(c1ncc3c(C)nn(CC(F)F)c3n1)CC2] | 874 |
| 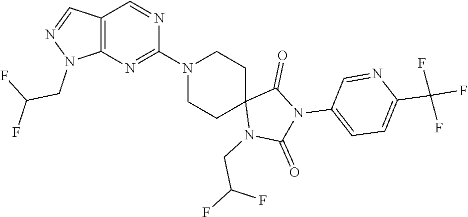 [SMILES: O=C1N(c2ccc(C(F)(F)F)nc2)C(=O)C2(CCN(c3ncc4cnn(CC(F)F)c4n3)CC2)N1CC(F)F] | 875 |
| 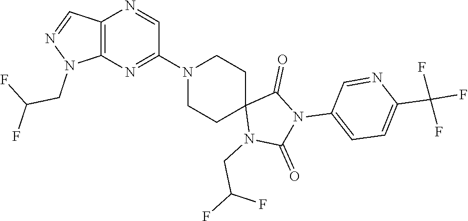 [SMILES: O=C1N(c2ccc(C(F)(F)F)nc2)C(=O)C2(CCN(c3cnc4cnn(CC(F)F)c4n3)CC2)N1CC(F)F] | 876 |
| 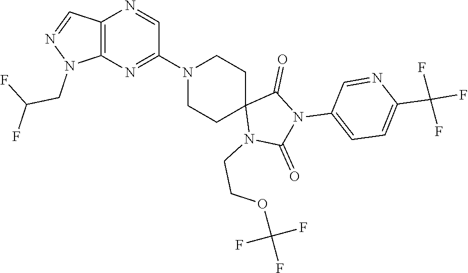 [SMILES: O=C1N(c2ccc(C(F)(F)F)nc2)C(=O)C2(CCN(c3cnc4cnn(CC(F)F)c4n3)CC2)N1CCOC(F)(F)F] | 877 |
| 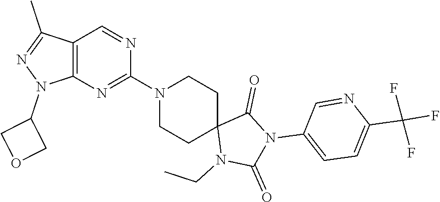 [SMILES: CCN1C(=O)N(c2ccc(C(F)(F)F)nc2)C(=O)C12CCN(c1ncc3c(C)nn(C4COC4)c3n1)CC2] | 878 |
| 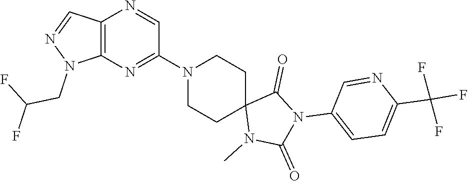 [SMILES: CN1C(=O)N(c2ccc(C(F)(F)F)nc2)C(=O)C12CCN(c1cnc3cnn(CC(F)F)c3n1)CC2] | 879 |
| 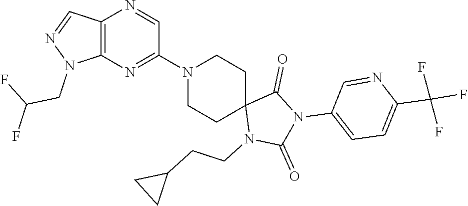 [SMILES: O=C1N(c2ccc(C(F)(F)F)nc2)C(=O)C2(CCN(c3cnc4cnn(CC(F)F)c4n3)CC2)N1CCC1CC1] | 880 |
| 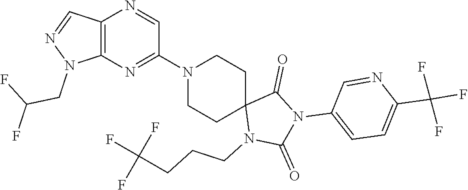 [SMILES: O=C1N(c2ccc(C(F)(F)F)nc2)C(=O)C2(CCN(c3cnc4cnn(CC(F)F)c4n3)CC2)N1CCCC(F)(F)F] | 881 |
| 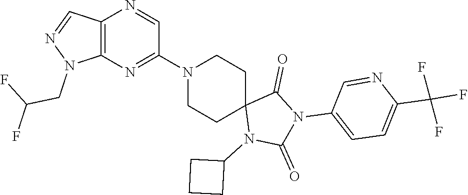 [SMILES: O=C1N(c2ccc(C(F)(F)F)nc2)C(=O)C2(CCN(c3cnc4cnn(CC(F)F)c4n3)CC2)N1C1CCC1] | 882 |
| 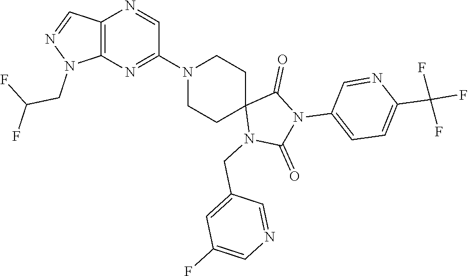 [SMILES: O=C1N(c2ccc(C(F)(F)F)nc2)C(=O)C2(CCN(c3cnc4cnn(CC(F)F)c4n3)CC2)N1Cc1cncc(F)c1] | 883 |
| 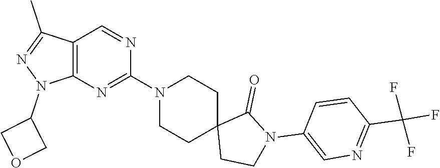 [SMILES: Cc1nn(C2COC2)c2nc(N3CCC4(CC3)CCN(c3ccc(C(F)(F)F)nc3)C4=O)ncc12] | 884 |
| 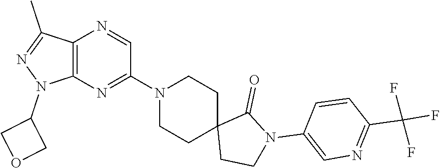 [SMILES: Cc1nn(C2COC2)c2nc(N3CCC4(CC3)CCN(c3ccc(C(F)(F)F)nc3)C4=O)cnc12] | 885 |
| 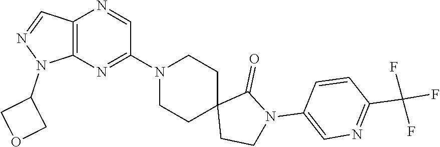 [SMILES: O=C1N(c2ccc(C(F)(F)F)nc2)CCC12CCN(c1cnc3cnn(C4COC4)c3n1)CC2] | 886 |
| 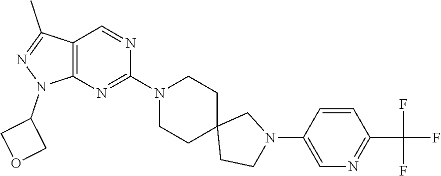 [SMILES: Cc1nn(C2COC2)c2nc(N3CCC4(CCN(c5ccc(C(F)(F)F)nc5)C4)CC3)ncc12] | 887 |
| 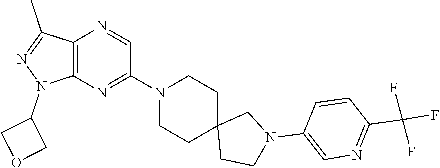 [SMILES: Cc1nn(C2COC2)c2nc(N3CCC4(CCN(c5ccc(C(F)(F)F)nc5)C4)CC3)cnc12] | 888 |
| 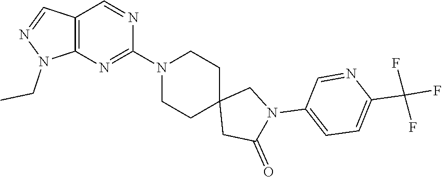 [SMILES: CCn1ncc2cnc(N3CCC4(CC3)CC(=O)N(c3ccc(C(F)(F)F)nc3)C4)nc21] | 889 |
| 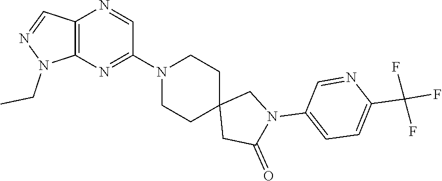 [SMILES: CCn1ncc2ncc(N3CCC4(CC3)CC(=O)N(c3ccc(C(F)(F)F)nc3)C4)nc21] | 890 |
| 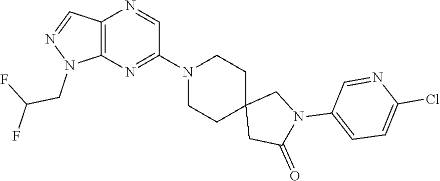 [SMILES: O=C1CC2(CCN(c3cnc4cnn(CC(F)F)c4n3)CC2)CN1c1ccc(Cl)nc1] | 891 |
| 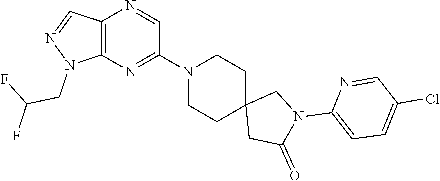 [SMILES: O=C1CC2(CCN(c3cnc4cnn(CC(F)F)c4n3)CC2)CN1c1ccc(Cl)cn1] | 892 |
| 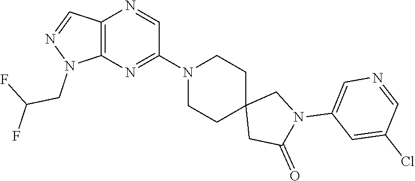 [SMILES: O=C1CC2(CCN(c3cnc4cnn(CC(F)F)c4n3)CC2)CN1c1cncc(Cl)c1] | 893 |
| 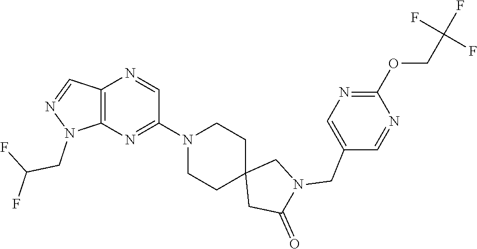 [SMILES: O=C1CC2(CCN(c3cnc4cnn(CC(F)F)c4n3)CC2)CN1Cc1cnc(OCC(F)(F)F)nc1] | 894 |
| 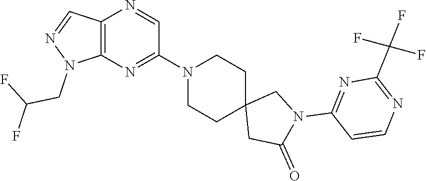 [SMILES: O=C1CC2(CCN(c3cnc4cnn(CC(F)F)c4n3)CC2)CN1c1ccnc(C(F)(F)F)n1] | 895 |
| 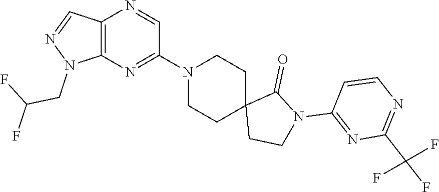 [SMILES: O=C1N(c2ccnc(C(F)(F)F)n2)CCC12CCN(c1cnc3cnn(CC(F)F)c3n1)CC2] | 896 |
| 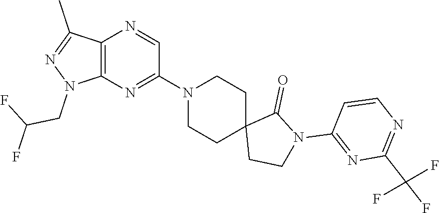 [SMILES: Cc1nn(CC(F)F)c2nc(N3CCC4(CC3)CCN(c3ccnc(C(F)(F)F)n3)C4=O)cnc12] | 897 |
| 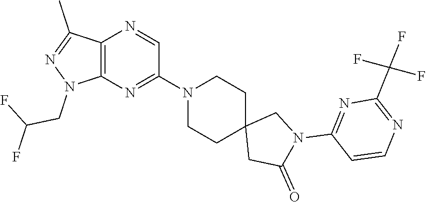 [SMILES: Cc1nn(CC(F)F)c2nc(N3CCC4(CC3)CC(=O)N(c3ccnc(C(F)(F)F)n3)C4)cnc12] | 898 |
| 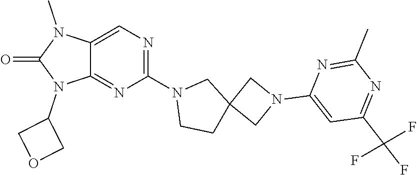 [SMILES: Cc1nc(N2CC3(CCN(c4ncc5c(n4)n(C4COC4)c(=O)n5C)C3)C2)cc(C(F)(F)F)n1] | 899 |
| 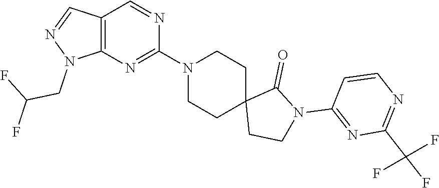 [SMILES: O=C1N(c2ccnc(C(F)(F)F)n2)CCC12CCN(c1ncc3cnn(CC(F)F)c3n1)CC2] | 900 |
| 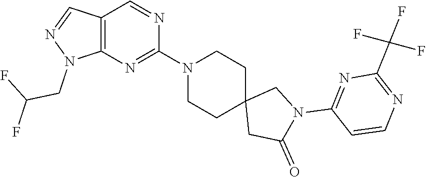 [SMILES: O=C1CC2(CCN(c3ncc4cnn(CC(F)F)c4n3)CC2)CN1c1ccnc(C(F)(F)F)n1] | 901 |
| 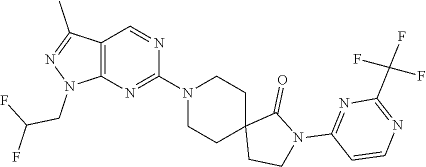 [SMILES: Cc1nn(CC(F)F)c2nc(N3CCC4(CC3)CCN(c3ccnc(C(F)(F)F)n3)C4=O)ncc12] | 902 |
| 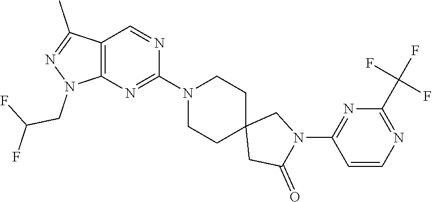 [SMILES: Cc1nn(CC(F)F)c2nc(N3CCC4(CC3)CC(=O)N(c3ccnc(C(F)(F)F)n3)C4)ncc12] | 903 |
| 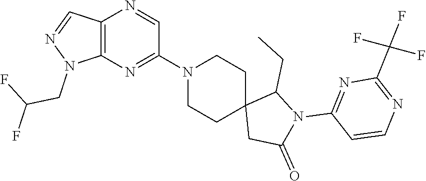 [SMILES: CCC1N(c2ccnc(C(F)(F)F)n2)C(=O)CC12CCN(c1cnc3cnn(CC(F)F)c3n1)CC2] | 904 |
| 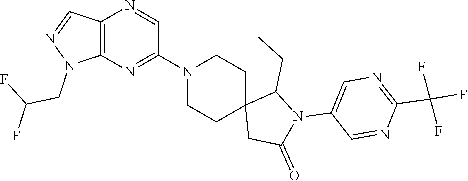 [SMILES: CCC1N(c2cnc(C(F)(F)F)nc2)C(=O)CC12CCN(c1cnc3cnn(CC(F)F)c3n1)CC2] | 905 |
| 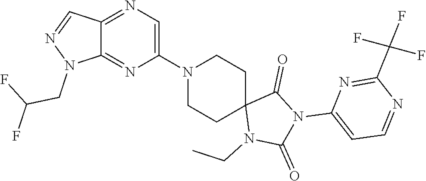 [SMILES: CCN1C(=O)N(c2ccnc(C(F)(F)F)n2)C(=O)C12CCN(c1cnc3cnn(CC(F)F)c3n1)CC2] | 906 |
| 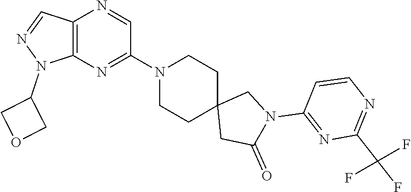 [SMILES: O=C1CC2(CCN(c3cnc4cnn(C5COC5)c4n3)CC2)CN1c1ccnc(C(F)(F)F)n1] | 907 |
| 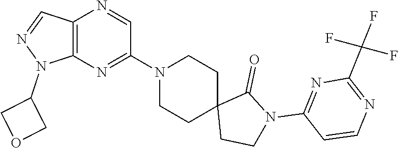 [SMILES: O=C1N(c2ccnc(C(F)(F)F)n2)CCC12CCN(c1cnc3cnn(C4COC4)c3n1)CC2] | 908 |
| 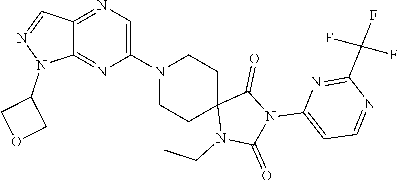 [SMILES: CCN1C(=O)N(c2ccnc(C(F)(F)F)n2)C(=O)C12CCN(c1cnc3cnn(C4COC4)c3n1)CC2] | 909 |
| 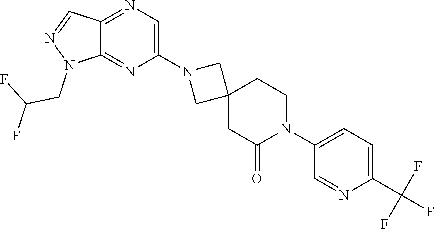 [SMILES: O=C1CC2(CCN1c1ccc(C(F)(F)F)nc1)CN(c1cnc3cnn(CC(F)F)c3n1)C2] | 910 |
| 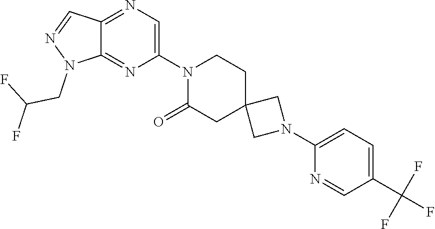 [SMILES: O=C1CC2(CCN1c1cnc3cnn(CC(F)F)c3n1)CN(c1ccc(C(F)(F)F)cn1)C2] | 911 |
| 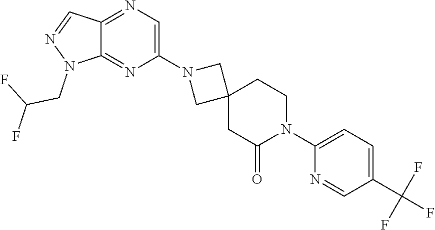 [SMILES: O=C1CC2(CCN1c1ccc(C(F)(F)F)cn1)CN(c1cnc3cnn(CC(F)F)c3n1)C2] | 912 |
| 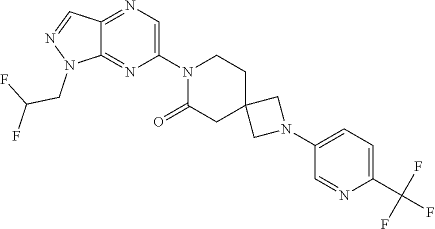 [SMILES: O=C1CC2(CCN1c1cnc3cnn(CC(F)F)c3n1)CN(c1ccc(C(F)(F)F)nc1)C2] | 913 |
| 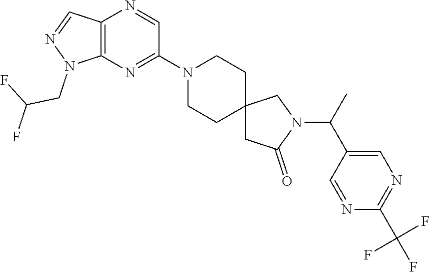 [SMILES: CC(c1cnc(C(F)(F)F)nc1)N1CC2(CCN(c3cnc4cnn(CC(F)F)c4n3)CC2)CC1=O] | 914 |
| 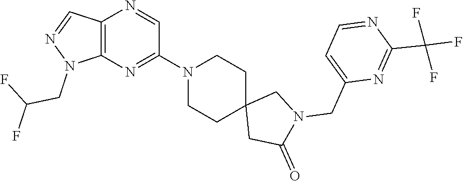 [SMILES: O=C1CC2(CCN(c3cnc4cnn(CC(F)F)c4n3)CC2)CN1Cc1ccnc(C(F)(F)F)n1] | 915 |
| 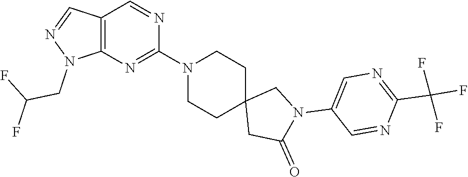 [SMILES: O=C1CC2(CCN(c3ncc4cnn(CC(F)F)c4n3)CC2)CN1c1cnc(C(F)(F)F)nc1] | 916 |
| 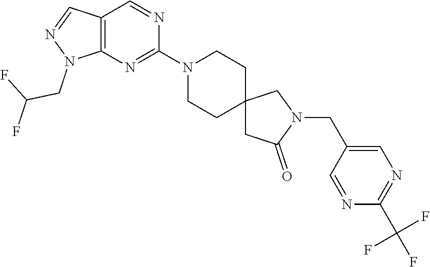 [SMILES: O=C1CC2(CCN(c3ncc4cnn(CC(F)F)c4n3)CC2)CN1Cc1cnc(C(F)(F)F)nc1] | 917 |
| 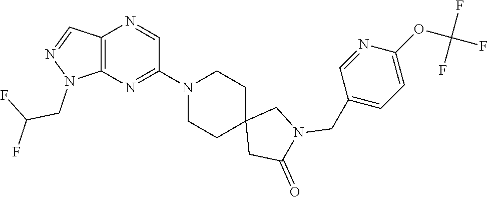 [SMILES: O=C1CC2(CCN(c3cnc4cnn(CC(F)F)c4n3)CC2)CN1Cc1ccc(OC(F)(F)F)nc1] | 918 |
| 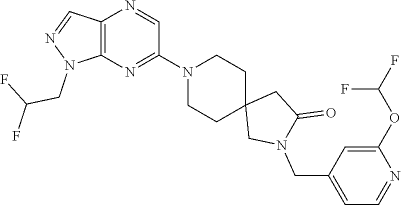 [SMILES: O=C1CC2(CCN(c3cnc4cnn(CC(F)F)c4n3)CC2)CN1Cc1ccnc(OC(F)F)c1] | 919 |
| 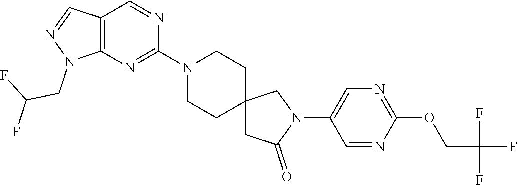 [SMILES: O=C1CC2(CCN(c3ncc4cnn(CC(F)F)c4n3)CC2)CN1c1cnc(OCC(F)(F)F)nc1] | 920 |
| 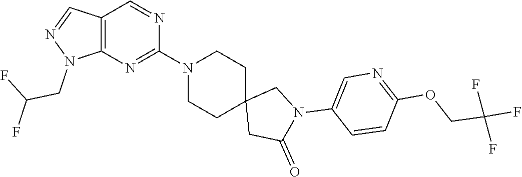 [SMILES: O=C1CC2(CCN(c3ncc4cnn(CC(F)F)c4n3)CC2)CN1c1ccc(OCC(F)(F)F)nc1] | 921 |
| 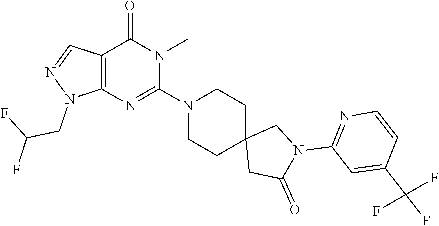 [SMILES: Cn1c(N2CCC3(CC2)CC(=O)N(c2cc(C(F)(F)F)ccn2)C3)nc2c(cnn2CC(F)F)c1=O] | 922 |
| 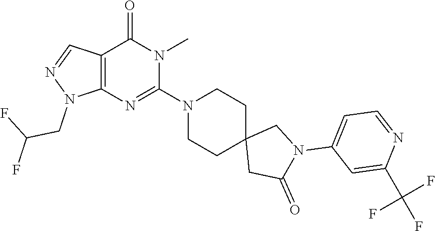 [SMILES: Cn1c(N2CCC3(CC2)CC(=O)N(c2ccnc(C(F)(F)F)c2)C3)nc2c(cnn2CC(F)F)c1=O] | 923 |
| 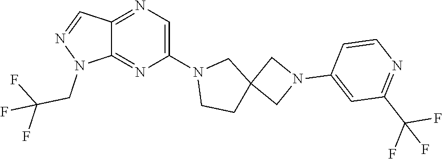 [SMILES: FC(F)(F)Cn1ncc2ncc(N3CCC4(CN(c5ccnc(C(F)(F)F)c5)C4)C3)nc21] | 924 |
| 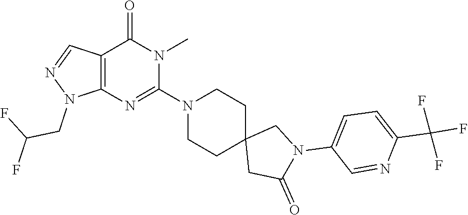 [SMILES: Cn1c(N2CCC3(CC2)CC(=O)N(c2ccc(C(F)(F)F)nc2)C3)nc2c(cnn2CC(F)F)c1=O] | 925 |
| 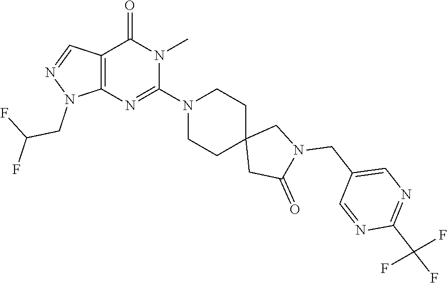 [SMILES: Cn1c(N2CCC3(CC2)CC(=O)N(Cc2cnc(C(F)(F)F)nc2)C3)nc2c(cnn2CC(F)F)c1=O] | 926 |
| 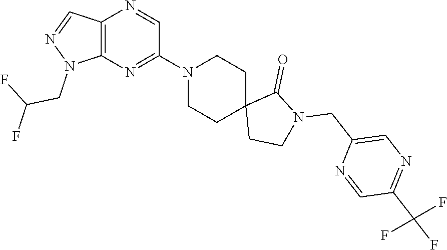 [SMILES: O=C1N(Cc2cnc(C(F)(F)F)cn2)CCC12CCN(c1cnc3cnn(CC(F)F)c3n1)CC2] | 927 |
| 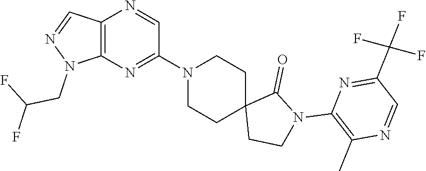 [SMILES: Cc1ncc(C(F)(F)F)nc1N1CCC2(CCN(c3cnc4cnn(CC(F)F)c4n3)CC2)C1=O] | 928 |
| 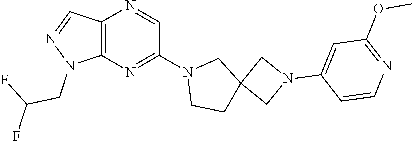 [SMILES: COc1cc(N2CC3(CCN(c4cnc5cnn(CC(F)F)c5n4)C3)C2)ccn1] | 929 |
| 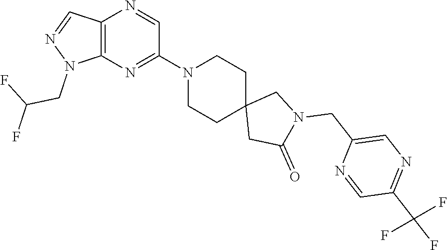 [SMILES: O=C1CC2(CCN(c3cnc4cnn(CC(F)F)c4n3)CC2)CN1Cc1cnc(C(F)(F)F)cn1] | 930 |
| 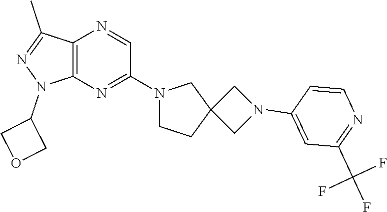 [SMILES: Cc1nn(C2COC2)c2nc(N3CCC4(CN(c5ccnc(C(F)(F)F)c5)C4)C3)cnc12] | 931 |
| 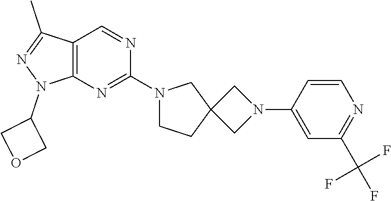 [SMILES: Cc1nn(C2COC2)c2nc(N3CCC4(CN(c5ccnc(C(F)(F)F)c5)C4)C3)ncc12] | 932 |
| 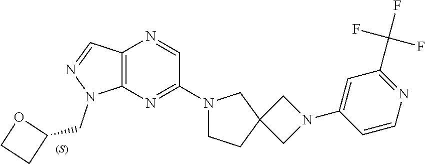 | 933 |
| 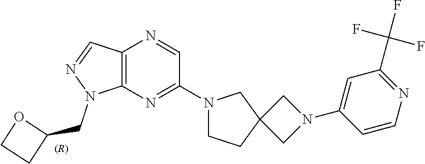 | 934 |
| 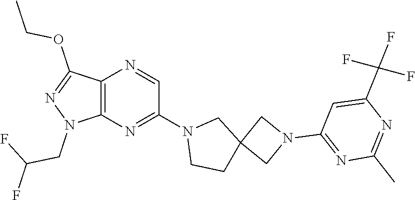 [SMILES: CCOc1nn(CC(F)F)c2nc(N3CCC4(CN(c5cc(C(F)(F)F)nc(C)n5)C4)C3)cnc12] | 935 |
| 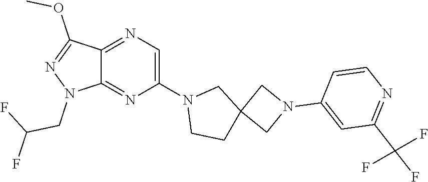 [SMILES: COc1nn(CC(F)F)c2nc(N3CCC4(CN(c5ccnc(C(F)(F)F)c5)C4)C3)cnc12] | 936 |
| 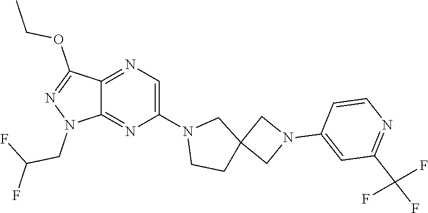 [SMILES: CCOc1nn(CC(F)F)c2nc(N3CCC4(CN(c5ccnc(C(F)(F)F)c5)C4)C3)cnc12] | 937 |
| 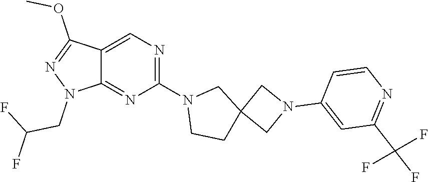 [SMILES: COc1nn(CC(F)F)c2nc(N3CCC4(CN(c5ccnc(C(F)(F)F)c5)C4)C3)ncc12] | 938 |
| 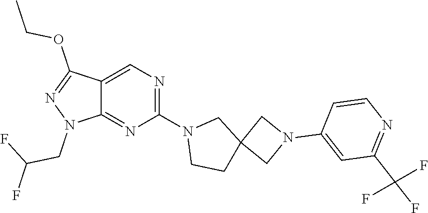 [SMILES: CCOc1nn(CC(F)F)c2nc(N3CCC4(CN(c5ccnc(C(F)(F)F)c5)C4)C3)ncc12] | 939 |
| 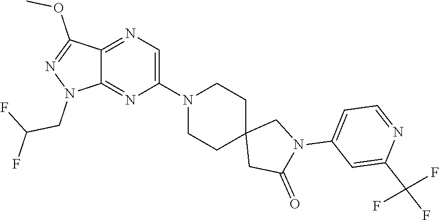 [SMILES: COc1nn(CC(F)F)c2nc(N3CCC4(CC3)CC(=O)N(c3ccnc(C(F)(F)F)c3)C4)cnc12] | 940 |
| 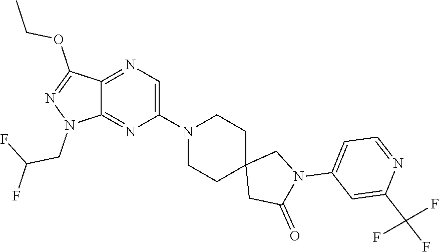 [SMILES: CCOc1nn(CC(F)F)c2nc(N3CCC4(CC3)CC(=O)N(c3ccnc(C(F)(F)F)c3)C4)cnc12] | 941 |
| 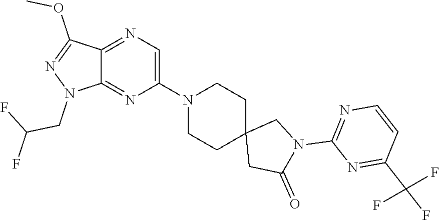 [SMILES: COc1nn(CC(F)F)c2nc(N3CCC4(CC3)CC(=O)N(c3nccc(C(F)(F)F)n3)C4)cnc12] | 942 |
| 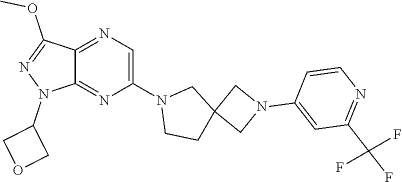 [SMILES: COc1nn(C2COC2)c2nc(N3CCC4(CN(c5ccnc(C(F)(F)F)c5)C4)C3)cnc12] | 943 |
| 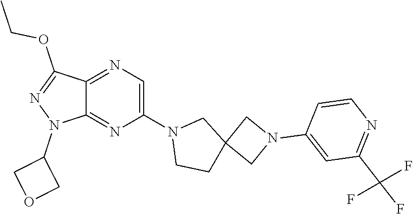 [SMILES: CCOc1nn(C2COC2)c2nc(N3CCC4(CN(c5ccnc(C(F)(F)F)c5)C4)C3)cnc12] | 944 |
| 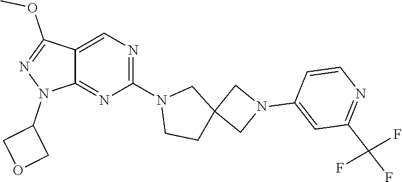 [SMILES: COc1nn(C2COC2)c2nc(N3CCC4(CN(c5ccnc(C(F)(F)F)c5)C4)C3)ncc12] | 945 |
| 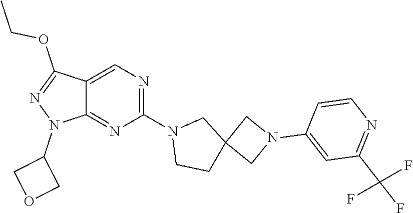 [SMILES: CCOc1nn(C2COC2)c2nc(N3CCC4(CN(c5ccnc(C(F)(F)F)c5)C4)C3)ncc12] | 946 |
| 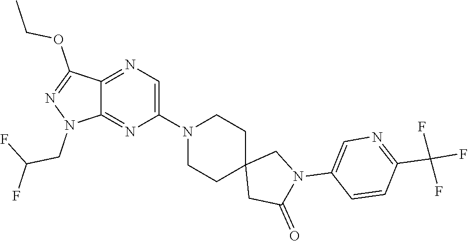 [SMILES: CCOc1nn(CC(F)F)c2nc(N3CCC4(CC3)CC(=O)N(c3ccc(C(F)(F)F)nc3)C4)cnc12] | 947 |
| 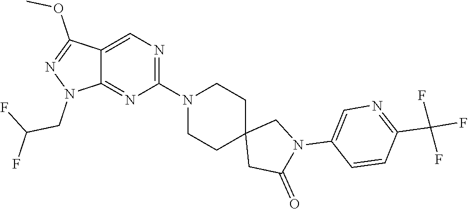 [SMILES: COc1nn(CC(F)F)c2nc(N3CCC4(CC3)CC(=O)N(c3ccc(C(F)(F)F)nc3)C4)ncc12] | 948 |
| 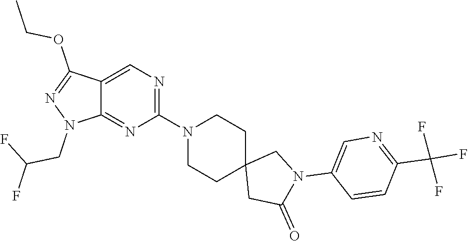 [SMILES: CCOc1nn(CC(F)F)c2nc(N3CCC4(CC3)CC(=O)N(c3ccc(C(F)(F)F)nc3)C4)ncc12] | 949 |
| 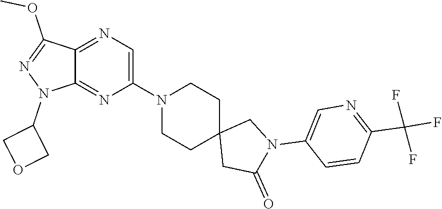 [SMILES: COc1nn(C2COC2)c2nc(N3CCC4(CC3)CC(=O)N(c3ccc(C(F)(F)F)nc3)C4)cnc12] | 950 |
| 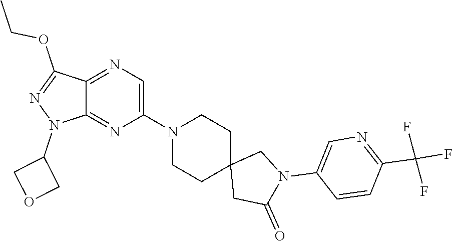 [SMILES: CCOc1nn(C2COC2)c2nc(N3CCC4(CC3)CC(=O)N(c3ccc(C(F)(F)F)nc3)C4)cnc12] | 951 |
| 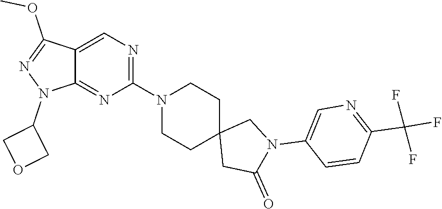 [SMILES: COc1nn(C2COC2)c2nc(N3CCC4(CC3)CC(=O)N(c3ccc(C(F)(F)F)nc3)C4)ncc12] | 952 |
| 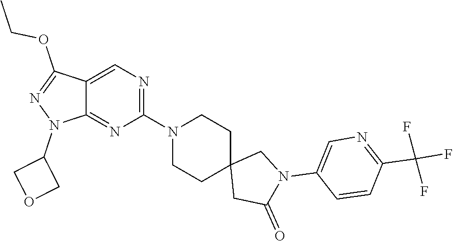 [SMILES: CCOc1nn(C2COC2)c2nc(N3CCC4(CC3)CC(=O)N(c3ccc(C(F)(F)F)nc3)C4)ncc12] | 953 |
| 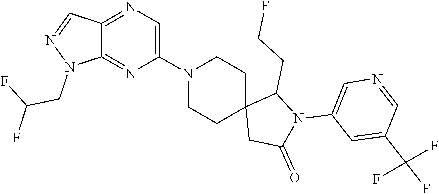 [SMILES: O=C1CC2(CCN(c3cnc4cnn(CC(F)F)c4n3)CC2)C(CCF)N1c1cncc(C(F)(F)F)c1] | 954 |
| 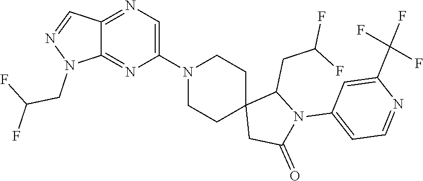 [SMILES: O=C1CC2(CCN(c3cnc4cnn(CC(F)F)c4n3)CC2)C(CC(F)F)N1c1ccnc(C(F)(F)F)c1] | 955 |
| 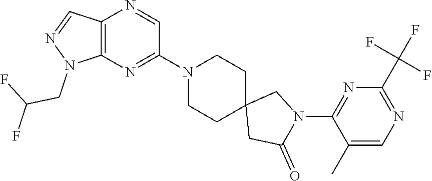 [SMILES: Cc1cnc(C(F)(F)F)nc1N1CC2(CCN(c3cnc4cnn(CC(F)F)c4n3)CC2)CC1=O] | 956 |
| 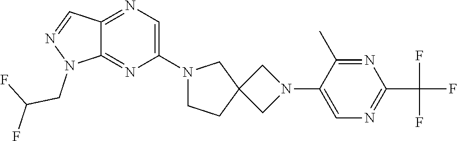 [SMILES: Cc1nc(C(F)(F)F)ncc1N1CC2(CCN(c3cnc4cnn(CC(F)F)c4n3)C2)C1] | 957 |
| 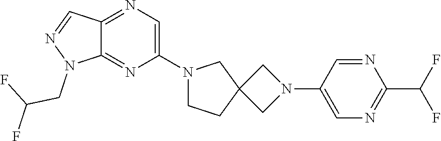 [SMILES: FC(F)Cn1ncc2ncc(N3CCC4(CN(c5cnc(C(F)F)nc5)C4)C3)nc21] | 958 |
| 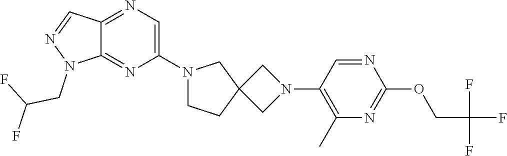 [SMILES: Cc1nc(OCC(F)(F)F)ncc1N1CC2(CCN(c3cnc4cnn(CC(F)F)c4n3)C2)C1] | 959 |
| 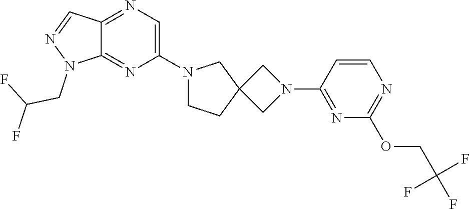 [SMILES: FC(F)Cn1ncc2ncc(N3CCC4(CN(c5ccnc(OCC(F)(F)F)n5)C4)C3)nc21] | 960 |
| 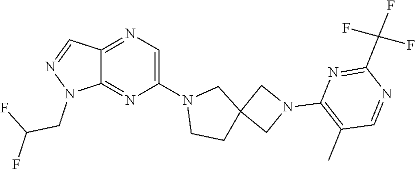 [SMILES: Cc1cnc(C(F)(F)F)nc1N1CC2(CCN(c3cnc4cnn(CC(F)F)c4n3)C2)C1] | 961 |
| 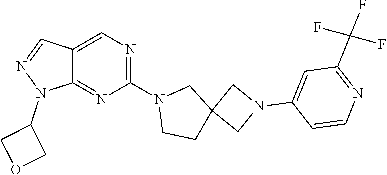 [SMILES: FC(F)(F)c1cc(N2CC3(CCN(c4ncc5cnn(C6COC6)c5n4)C3)C2)ccn1] | 962 |
| 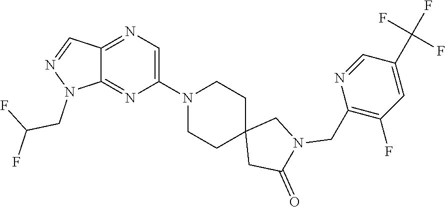 [SMILES: O=C1CC2(CCN(c3cnc4cnn(CC(F)F)c4n3)CC2)CN1Cc1ncc(C(F)(F)F)cc1F] | 963 |
| 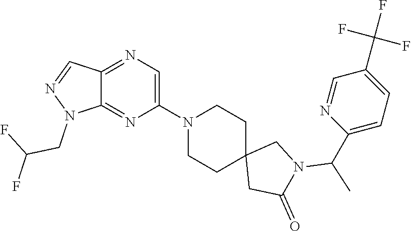 [SMILES: CC(c1ccc(C(F)(F)F)cn1)N1CC2(CCN(c3cnc4cnn(CC(F)F)c4n3)CC2)CC1=O] | 964 |
| 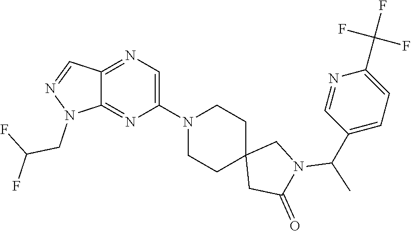 [SMILES: CC(c1ccc(C(F)(F)F)nc1)N1CC2(CCN(c3cnc4cnn(CC(F)F)c4n3)CC2)CC1=O] | 965 |
| 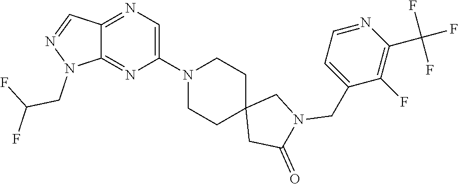 [SMILES: O=C1CC2(CCN(c3cnc4cnn(CC(F)F)c4n3)CC2)CN1Cc1ccnc(C(F)(F)F)c1F] | 966 |
| 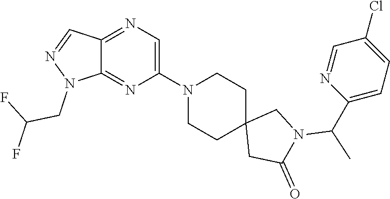 [SMILES: CC(c1ccc(Cl)cn1)N1CC2(CCN(c3cnc4cnn(CC(F)F)c4n3)CC2)CC1=O] | 967 |
| 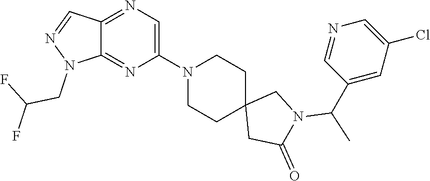 [SMILES: CC(c1cncc(Cl)c1)N1CC2(CCN(c3cnc4cnn(CC(F)F)c4n3)CC2)CC1=O] | 968 |
| 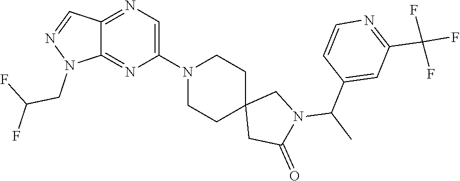 [SMILES: CC(c1ccnc(C(F)(F)F)c1)N1CC2(CCN(c3cnc4cnn(CC(F)F)c4n3)CC2)CC1=O] | 969 |
| 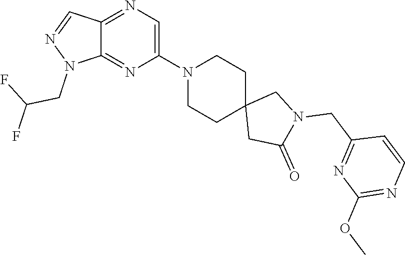 [SMILES: COc1nccc(CN2CC3(CCN(c4cnc5cnn(CC(F)F)c5n4)CC3)CC2=O)n1] | 970 |
| 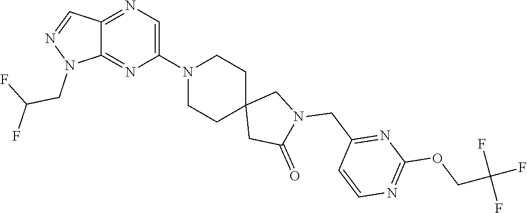 [SMILES: O=C1CC2(CCN(c3cnc4cnn(CC(F)F)c4n3)CC2)CN1Cc1ccnc(OCC(F)(F)F)n1] | 971 |
| 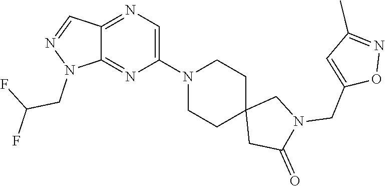 [SMILES: Cc1cc(CN2CC3(CCN(c4cnc5cnn(CC(F)F)c5n4)CC3)CC2=O)on1] | 972 |
| 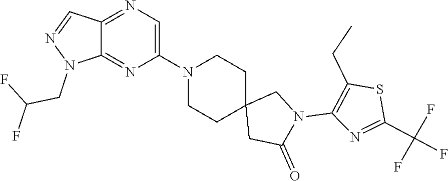 [SMILES: CCc1sc(C(F)(F)F)nc1N1CC2(CCN(c3cnc4cnn(CC(F)F)c4n3)CC2)CC1=O] | 973 |
| 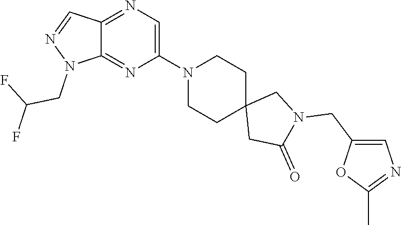 [SMILES: Cc1ncc(CN2CC3(CCN(c4cnc5cnn(CC(F)F)c5n4)CC3)CC2=O)o1] | 974 |
| 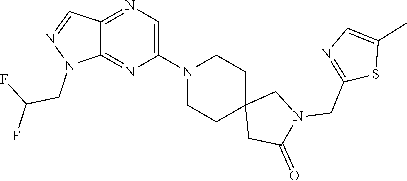 [SMILES: Cc1cnc(CN2CC3(CCN(c4cnc5cnn(CC(F)F)c5n4)CC3)CC2=O)s1] | 975 |
| 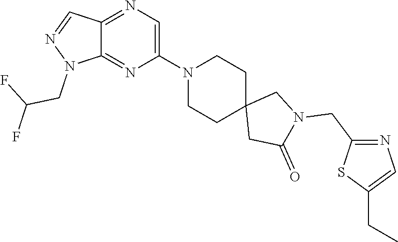 [SMILES: CCc1cnc(CN2CC3(CCN(c4cnc5cnn(CC(F)F)c5n4)CC3)CC2=O)s1] | 976 |
| 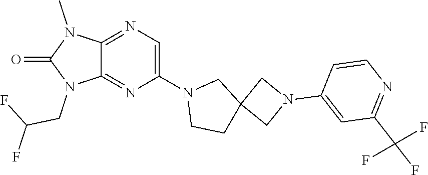 [SMILES: Cn1c(=O)n(CC(F)F)c2nc(N3CCC4(CN(c5ccnc(C(F)(F)F)c5)C4)C3)cnc21] | 977 |
| 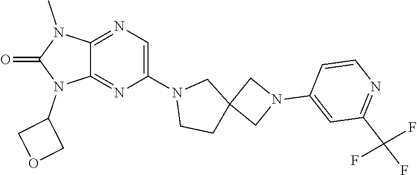 [SMILES: Cn1c(=O)n(C2COC2)c2nc(N3CCC4(CN(c5ccnc(C(F)(F)F)c5)C4)C3)cnc21] | 978 |
| 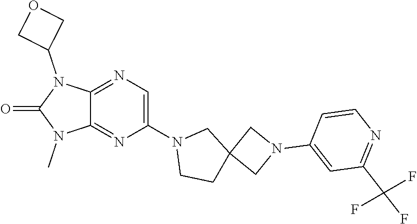 [SMILES: Cn1c(=O)n(C2COC2)c2ncc(N3CCC4(CN(c5ccnc(C(F)(F)F)c5)C4)C3)nc21] | 979 |
| 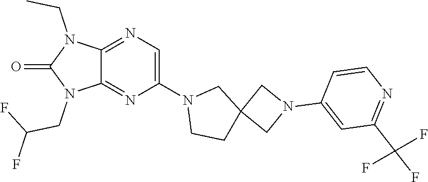 [SMILES: CCn1c(=O)n(CC(F)F)c2nc(N3CCC4(CN(c5ccnc(C(F)(F)F)c5)C4)C3)cnc21] | 980 |
| 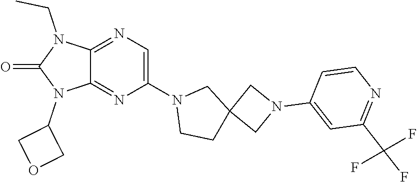 [SMILES: CCn1c(=O)n(C2COC2)c2nc(N3CCC4(CN(c5ccnc(C(F)(F)F)c5)C4)C3)cnc21] | 981 |
| 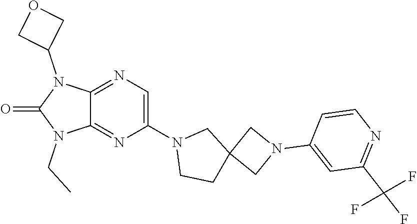 [SMILES: CCn1c(=O)n(C2COC2)c2ncc(N3CCC4(CN(c5ccnc(C(F)(F)F)c5)C4)C3)nc21] | 982 |
| 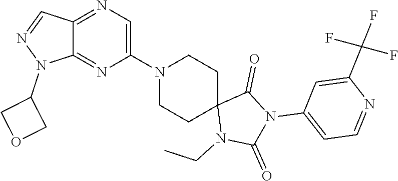 [SMILES: CCN1C(=O)N(c2ccnc(C(F)(F)F)c2)C(=O)C12CCN(c1cnc3cnn(C4COC4)c3n1)CC2] | 983 |
| 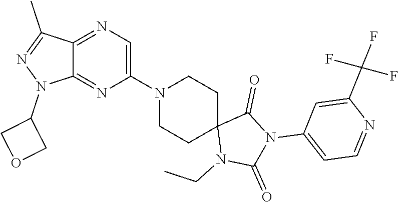 [SMILES: CCN1C(=O)N(c2ccnc(C(F)(F)F)c2)C(=O)C12CCN(c1cnc3c(C)nn(C4COC4)c3n1)CC2] | 984 |
| 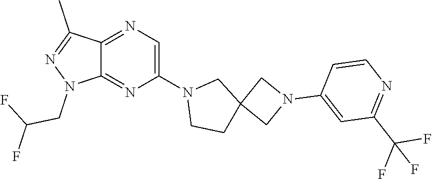 [SMILES: Cc1nn(CC(F)F)c2nc(N3CCC4(CN(c5ccnc(C(F)(F)F)c5)C4)C3)cnc12] | 985 |
| 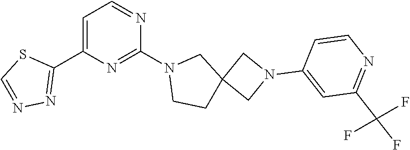 [SMILES: FC(F)(F)c1cc(N2CC3(CCN(c4nccc(-c5nncs5)n4)C3)C2)ccn1] | 986 |
| 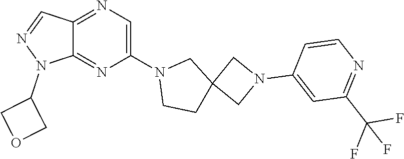 [SMILES: FC(F)(F)c1cc(N2CC3(CCN(c4cnc5cnn(C6COC6)c5n4)C3)C2)ccn1] | 987 |
| 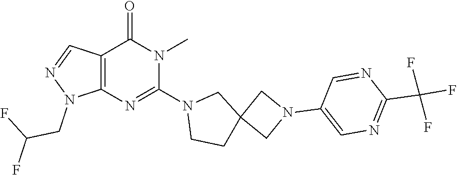 [SMILES: Cn1c(N2CCC3(CN(c4cnc(C(F)(F)F)nc4)C3)C2)nc2c(cnn2CC(F)F)c1=O] | 988 |
| 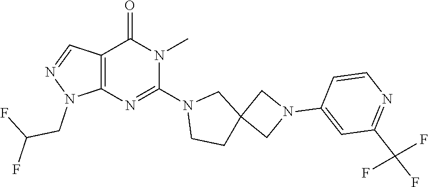 [SMILES: Cn1c(N2CCC3(CN(c4ccnc(C(F)(F)F)c4)C3)C2)nc2c(cnn2CC(F)F)c1=O] | 989 |
| 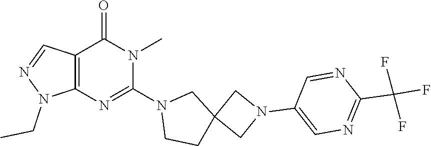 [SMILES: CCn1ncc2c(=O)n(C)c(N3CCC4(CN(c5cnc(C(F)(F)F)nc5)C4)C3)nc21] | 990 |
| 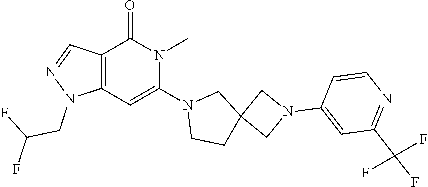 [SMILES: Cn1c(N2CCC3(CN(c4ccnc(C(F)(F)F)c4)C3)C2)cc2c(cnn2CC(F)F)c1=O] | 991 |
| 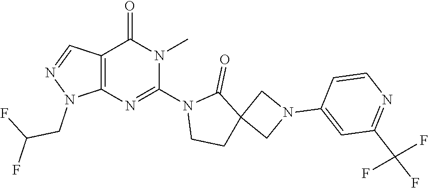 [SMILES: Cn1c(N2CCC3(CN(c4ccnc(C(F)(F)F)c4)C3)C2=O)nc2c(cnn2CC(F)F)c1=O] | 992 |
| 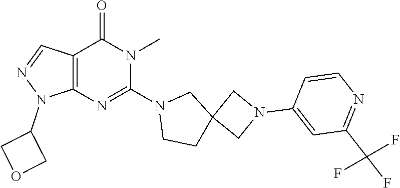 [SMILES: Cn1c(N2CCC3(CN(c4ccnc(C(F)(F)F)c4)C3)C2)nc2c(cnn2C2COC2)c1=O] | 993 |
| 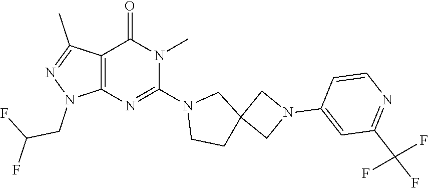 [SMILES: Cc1nn(CC(F)F)c2nc(N3CCC4(CN(c5ccnc(C(F)(F)F)c5)C4)C3)n(C)c(=O)c12] | 994 |
| 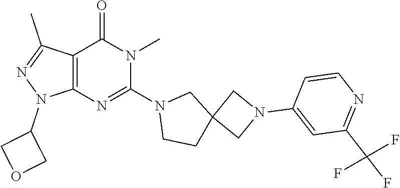 [SMILES: Cc1nn(C2COC2)c2nc(N3CCC4(CN(c5ccnc(C(F)(F)F)c5)C4)C3)n(C)c(=O)c12] | 995 |
| 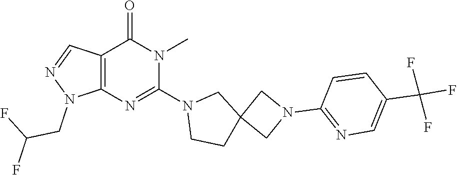 [SMILES: Cn1c(N2CCC3(CN(c4ccc(C(F)(F)F)cn4)C3)C2)nc2c(cnn2CC(F)F)c1=O] | 996 |
| 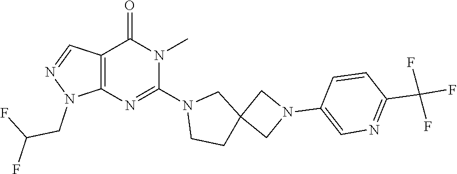 [SMILES: Cn1c(N2CCC3(CN(c4ccc(C(F)(F)F)nc4)C3)C2)nc2c(cnn2CC(F)F)c1=O] | 997 |
| 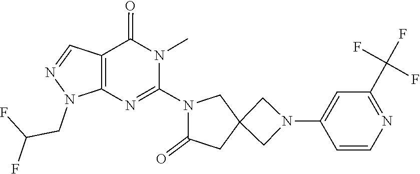 [SMILES: Cn1c(N2CC3(CC2=O)CN(c2ccnc(C(F)(F)F)c2)C3)nc2c(cnn2CC(F)F)c1=O] | 998 |
| 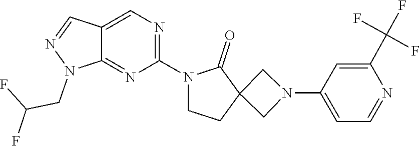 [SMILES: O=C1N(c2ncc3cnn(CC(F)F)c3n2)CCC12CN(c1ccnc(C(F)(F)F)c1)C2] | 999 |
| 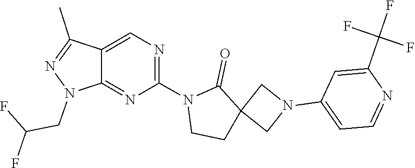 [SMILES: Cc1nn(CC(F)F)c2nc(N3CCC4(CN(c5ccnc(C(F)(F)F)c5)C4)C3=O)ncc12] | 1000 |
| 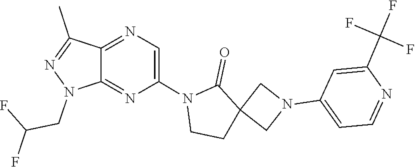 [SMILES: Cc1nn(CC(F)F)c2nc(N3CCC4(CN(c5ccnc(C(F)(F)F)c5)C4)C3=O)cnc12] | 1001 |
| 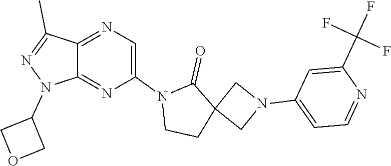 [SMILES: Cc1nn(C2COC2)c2nc(N3CCC4(CN(c5ccnc(C(F)(F)F)c5)C4)C3=O)cnc12] | 1002 |
| 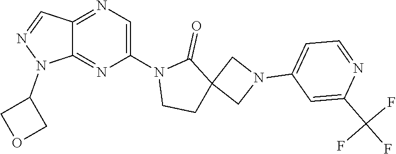 [SMILES: O=C1N(c2cnc3cnn(C4COC4)c3n2)CCC12CN(c1ccnc(C(F)(F)F)c1)C2] | 1003 |
| 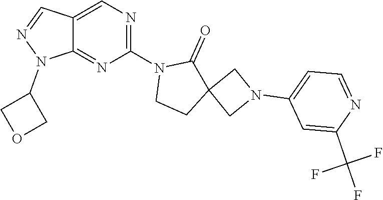 [SMILES: O=C1N(c2ncc3cnn(C4COC4)c3n2)CCC12CN(c1ccnc(C(F)(F)F)c1)C2] | 1004 |
| 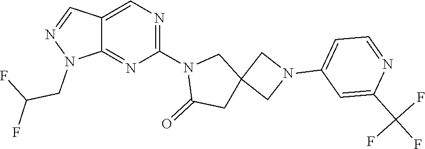 [SMILES: O=C1CC2(CN(c3ccnc(C(F)(F)F)c3)C2)CN1c1ncc2cnn(CC(F)F)c2n1] | 1005 |
| 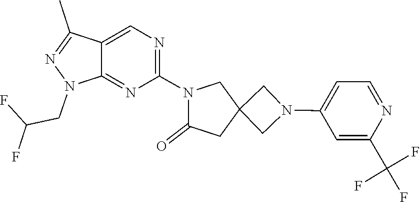 [SMILES: Cc1nn(CC(F)F)c2nc(N3CC4(CC3=O)CN(c3ccnc(C(F)(F)F)c3)C4)ncc12] | 1006 |
| 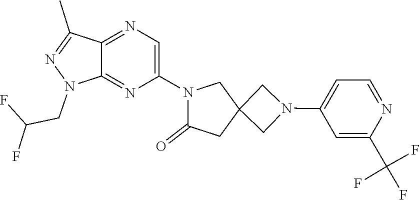 [SMILES: Cc1nn(CC(F)F)c2nc(N3CC4(CC3=O)CN(c3ccnc(C(F)(F)F)c3)C4)cnc12] | 1007 |
| 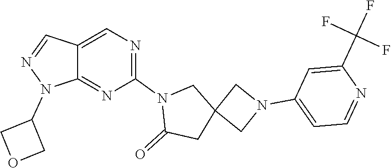 [SMILES: O=C1CC2(CN(c3ccnc(C(F)(F)F)c3)C2)CN1c1ncc2cnn(C3COC3)c2n1] | 1008 |
| 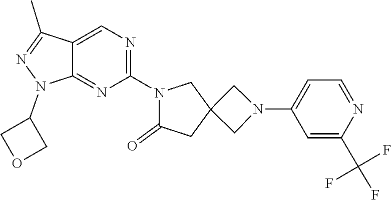 [SMILES: Cc1nn(C2COC2)c2nc(N3CC4(CC3=O)CN(c3ccnc(C(F)(F)F)c3)C4)ncc12] | 1009 |
| 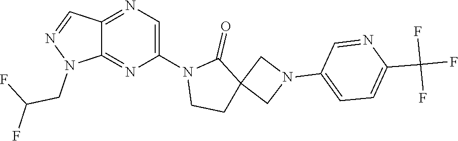 [SMILES: O=C1N(c2cnc3cnn(CC(F)F)c3n2)CCC12CN(c1ccc(C(F)(F)F)nc1)C2] | 1010 |
| 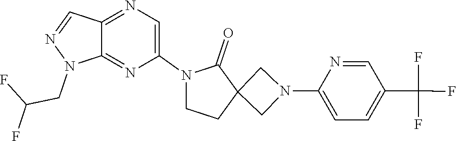 [SMILES: O=C1N(c2cnc3cnn(CC(F)F)c3n2)CCC12CN(c1ccc(C(F)(F)F)cn1)C2] | 1011 |
| 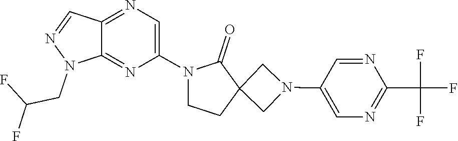 [SMILES: O=C1N(c2cnc3cnn(CC(F)F)c3n2)CCC12CN(c1cnc(C(F)(F)F)nc1)C2] | 1012 |
| 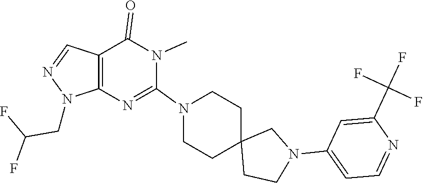 [SMILES: Cn1c(N2CCC3(CCN(c4ccnc(C(F)(F)F)c4)C3)CC2)nc2c(cnn2CC(F)F)c1=O] | 1013 |
| 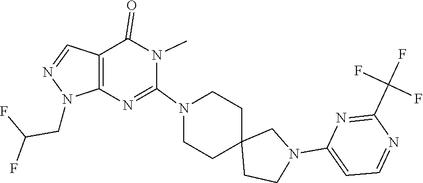 [SMILES: Cn1c(N2CCC3(CCN(c4ccnc(C(F)(F)F)n4)C3)CC2)nc2c(cnn2CC(F)F)c1=O] | 1014 |
| 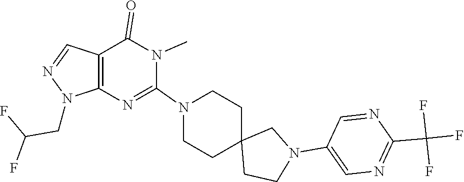 [SMILES: Cn1c(N2CCC3(CCN(c4cnc(C(F)(F)F)nc4)C3)CC2)nc2c(cnn2CC(F)F)c1=O] | 1015 |
| 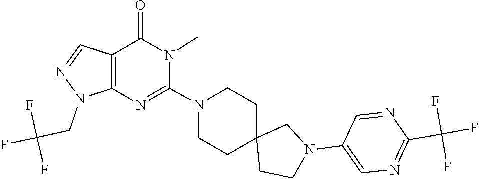 [SMILES: Cn1c(N2CCC3(CCN(c4cnc(C(F)(F)F)nc4)C3)CC2)nc2c(cnn2CC(F)(F)F)c1=O] | 1016 |
| 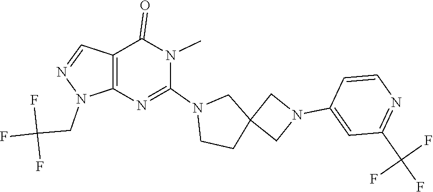 [SMILES: Cn1c(N2CCC3(CN(c4ccnc(C(F)(F)F)c4)C3)C2)nc2c(cnn2CC(F)(F)F)c1=O] | 1017 |
| 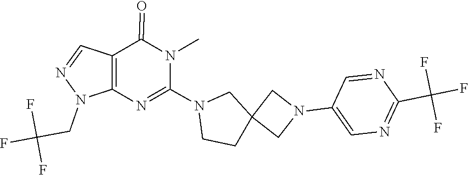 [SMILES: Cn1c(N2CCC3(CN(c4cnc(C(F)(F)F)nc4)C3)C2)nc2c(cnn2CC(F)(F)F)c1=O] | 1018 |
| 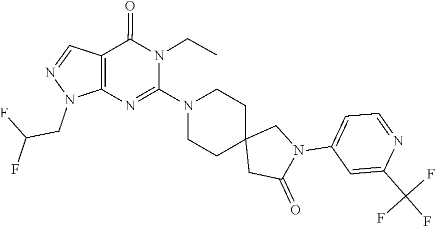 [SMILES: CCn1c(N2CCC3(CC2)CC(=O)N(c2ccnc(C(F)(F)F)c2)C3)nc2c(cnn2CC(F)F)c1=O] | 1019 |
| 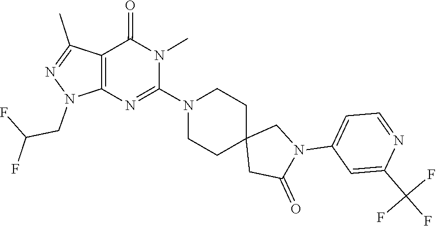 [SMILES: Cc1nn(CC(F)F)c2nc(N3CCC4(CC3)CC(=O)N(c3ccnc(C(F)(F)F)c3)C4)n(C)c(=O)c12] | 1020 |
| 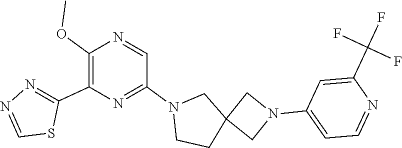 [SMILES: COc1ncc(N2CCC3(CN(c4ccnc(C(F)(F)F)c4)C3)C2)nc1-c1nncs1] | 1021 |
| 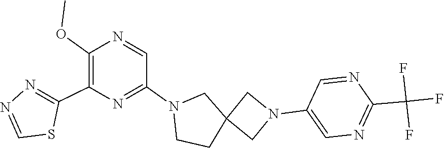 [SMILES: COc1ncc(N2CCC3(CN(c4cnc(C(F)(F)F)nc4)C3)C2)nc1-c1nncs1] | 1022 |
| 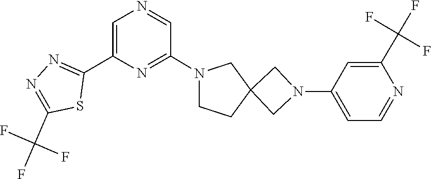 [SMILES: FC(F)(F)c1cc(N2CC3(CCN(c4cncc(-c5nnc(C(F)(F)F)s5)n4)C3)C2)ccn1] | 1023 |
| 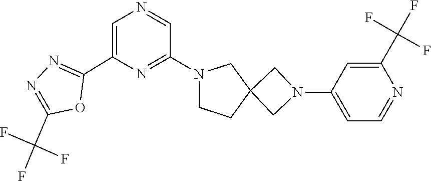 [SMILES: FC(F)(F)c1cc(N2CC3(CCN(c4cncc(-c5nnc(C(F)(F)F)o5)n4)C3)C2)ccn1] | 1024 |
| 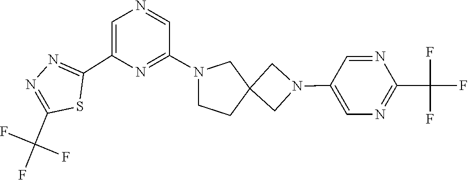 [SMILES: FC(F)(F)c1ncc(N2CC3(CCN(c4cncc(-c5nnc(C(F)(F)F)s5)n4)C3)C2)cn1] | 1025 |
| 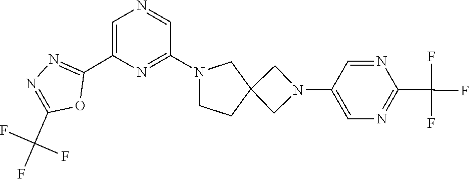 [SMILES: FC(F)(F)c1ncc(N2CC3(CCN(c4cncc(-c5nnc(C(F)(F)F)o5)n4)C3)C2)cn1] | 1026 |
| 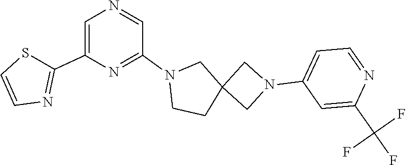 [SMILES: FC(F)(F)c1cc(N2CC3(CCN(c4cncc(-c5nccs5)n4)C3)C2)ccn1] | 1027 |
| 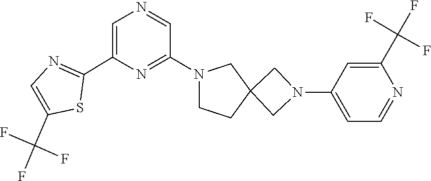 [SMILES: FC(F)(F)c1cc(N2CC3(CCN(c4cncc(-c5ncc(C(F)(F)F)s5)n4)C3)C2)ccn1] | 1028 |
| 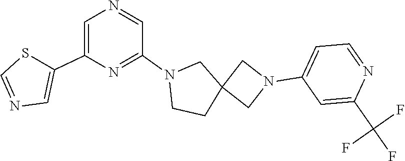 [SMILES: FC(F)(F)c1cc(N2CC3(CCN(c4cncc(-c5cncs5)n4)C3)C2)ccn1] | 1029 |
| 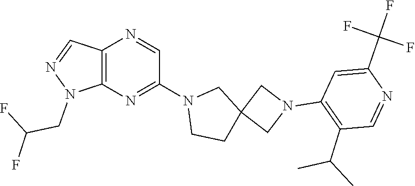 [SMILES: CC(C)c1cnc(C(F)(F)F)cc1N1CC2(CCN(c3cnc4cnn(CC(F)F)c4n3)C2)C1] | 1030 |
| 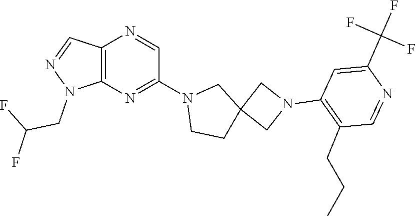 [SMILES: CCCc1cnc(C(F)(F)F)cc1N1CC2(CCN(c3cnc4cnn(CC(F)F)c4n3)C2)C1] | 1031 |
| 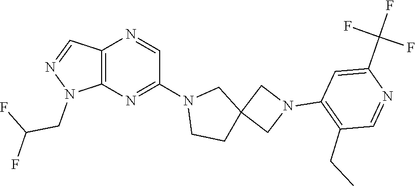 [SMILES: CCc1cnc(C(F)(F)F)cc1N1CC2(CCN(c3cnc4cnn(CC(F)F)c4n3)C2)C1] | 1032 |
| 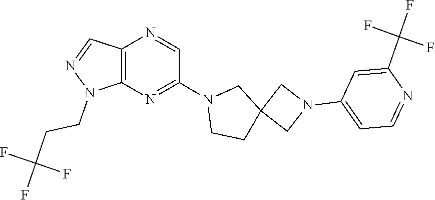 [SMILES: FC(F)(F)CCn1ncc2ncc(N3CCC4(CN(c5ccnc(C(F)(F)F)c5)C4)C3)nc21] | 1033 |
| 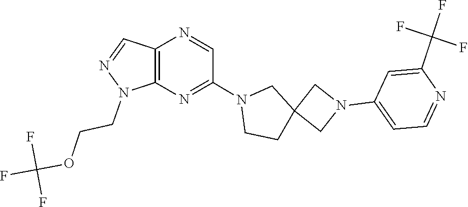 [SMILES: FC(F)(F)OCCn1ncc2ncc(N3CCC4(CN(c5ccnc(C(F)(F)F)c5)C4)C3)nc21] | 1034 |
| 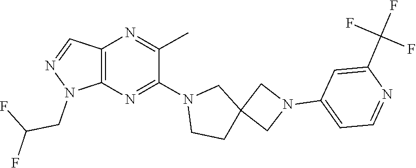 [SMILES: Cc1nc2cnn(CC(F)F)c2nc1N1CCC2(CN(c3ccnc(C(F)(F)F)c3)C2)C1] | 1035 |
| 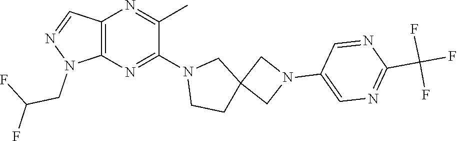 [SMILES: Cc1nc2cnn(CC(F)F)c2nc1N1CCC2(CN(c3cnc(C(F)(F)F)nc3)C2)C1] | 1036 |
| 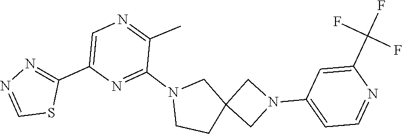 [SMILES: Cc1ncc(-c2nncs2)nc1N1CCC2(CN(c3ccnc(C(F)(F)F)c3)C2)C1] | 1037 |
| 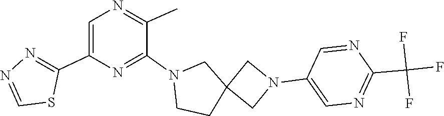 [SMILES: Cc1ncc(-c2nncs2)nc1N1CCC2(CN(c3cnc(C(F)(F)F)nc3)C2)C1] | 1038 |
| 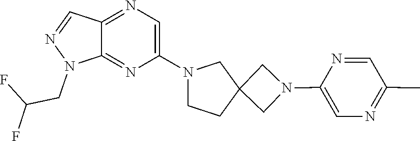 [SMILES: Cc1cnc(N2CC3(CCN(c4cnc5cnn(CC(F)F)c5n4)C3)C2)cn1] | 1039 |
| 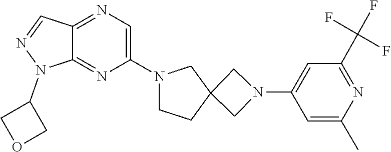 [SMILES: Cc1cc(N2CC3(CCN(c4cnc5cnn(C6COC6)c5n4)C3)C2)cc(C(F)(F)F)n1] | 1040 |
| 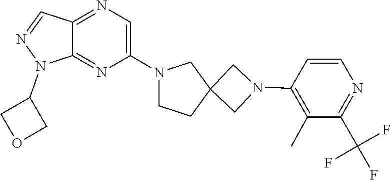 [SMILES: Cc1c(N2CC3(CCN(c4cnc5cnn(C6COC6)c5n4)C3)C2)ccnc1C(F)(F)F] | 1041 |
| 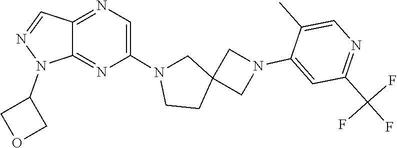 [SMILES: Cc1cnc(C(F)(F)F)cc1N1CC2(CCN(c3cnc4cnn(C5COC5)c4n3)C2)C1] | 1042 |
| 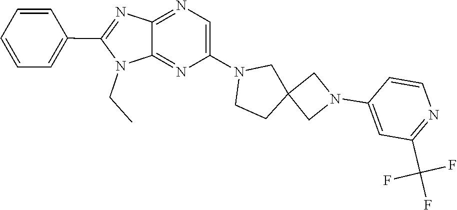 [SMILES: CCn1c(-c2ccccc2)nc2ncc(N3CCC4(CN(c5ccnc(C(F)(F)F)c5)C4)C3)nc21] | 1043 |
| 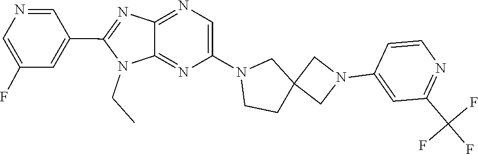 [SMILES: CCn1c(-c2cncc(F)c2)nc2ncc(N3CCC4(CN(c5ccnc(C(F)(F)F)c5)C4)C3)nc21] | 1044 |
| 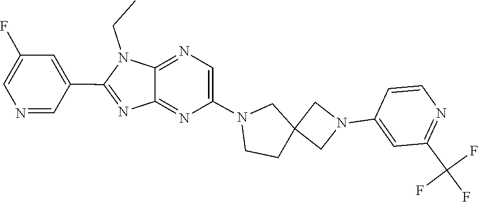 [SMILES: CCn1c(-c2cncc(F)c2)nc2nc(N3CCC4(CN(c5ccnc(C(F)(F)F)c5)C4)C3)cnc21] | 1045 |
| 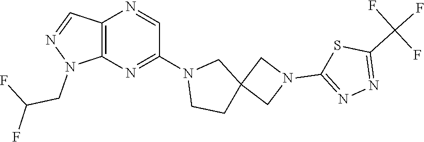 [SMILES: FC(F)Cn1ncc2ncc(N3CCC4(C3)CN(c3nnc(C(F)(F)F)s3)C4)nc21] | 1046 |
| 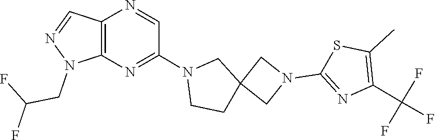 [SMILES: Cc1sc(N2CC3(CCN(c4cnc5cnn(CC(F)F)c5n4)C3)C2)nc1C(F)(F)F] | 1047 |
| 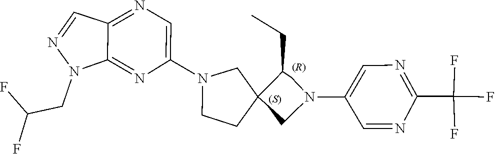 | 1048 |
| 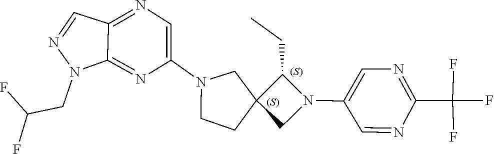 | 1049 |
| 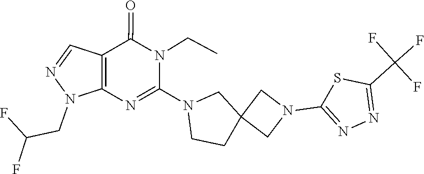 [SMILES: CCn1c(N2CCC3(CN(c4nnc(C(F)(F)F)s4)C3)C2)nc2c(cnn2CC(F)F)c1=O] | 1050 |
| 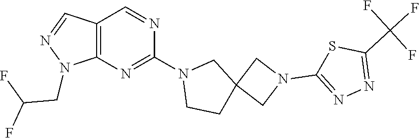 [SMILES: FC(F)Cn1ncc2cnc(N3CCC4(C3)CN(c3nnc(C(F)(F)F)s3)C4)nc21] | 1051 |
| 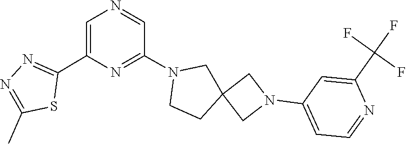 [SMILES: Cc1nnc(-c2cncc(N3CCC4(CN(c5ccnc(C(F)(F)F)c5)C4)C3)n2)s1] | 1052 |
| 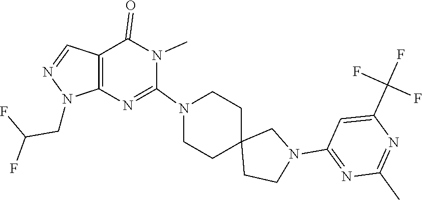 [SMILES: Cc1nc(N2CCC3(CCN(c4nc5c(cnn5CC(F)F)c(=O)n4C)CC3)C2)cc(C(F)(F)F)n1] | 1053 |
| 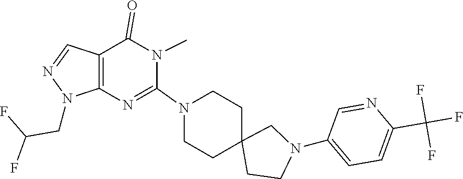 [SMILES: Cn1c(N2CCC3(CCN(c4ccc(C(F)(F)F)nc4)C3)CC2)nc2c(cnn2CC(F)F)c1=O] | 1054 |
| 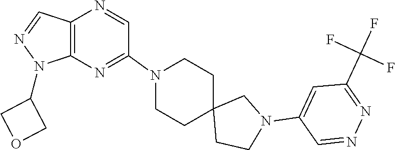 [SMILES: FC(F)(F)c1cc(N2CCC3(CCN(c4cnc5cnn(C6COC6)c5n4)CC3)C2)cnn1] | 1055 |
| 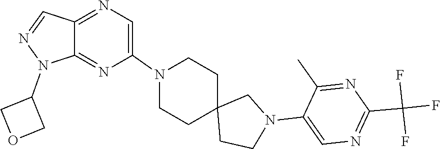 [SMILES: Cc1nc(C(F)(F)F)ncc1N1CCC2(CCN(c3cnc4cnn(C5COC5)c4n3)CC2)C1] | 1056 |
| 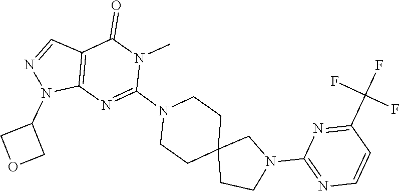 [SMILES: Cn1c(N2CCC3(CCN(c4nccc(C(F)(F)F)n4)C3)CC2)nc2c(cnn2C2COC2)c1=O] | 1057 |
| 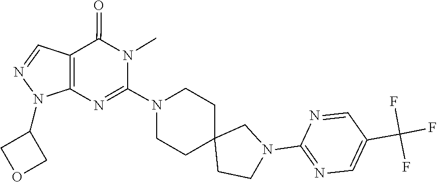 [SMILES: Cn1c(N2CCC3(CCN(c4ncc(C(F)(F)F)cn4)C3)CC2)nc2c(cnn2C2COC2)c1=O] | 1058 |
| 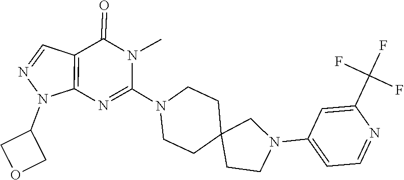 [SMILES: Cn1c(N2CCC3(CCN(c4ccnc(C(F)(F)F)c4)C3)CC2)nc2c(cnn2C2COC2)c1=O] | 1059 |
| 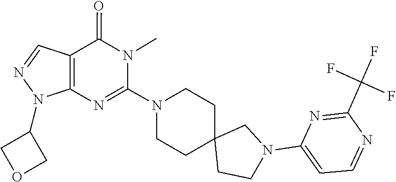 [SMILES: Cn1c(N2CCC3(CCN(c4ccnc(C(F)(F)F)n4)C3)CC2)nc2c(cnn2C2COC2)c1=O] | 1060 |
| 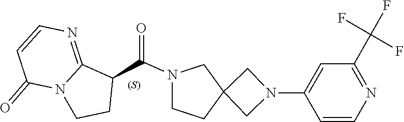 | 1061 |
| 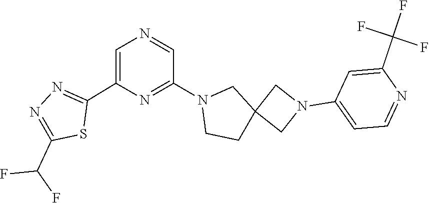 [SMILES: FC(F)c1nnc(-c2cncc(N3CCC4(CN(c5ccnc(C(F)(F)F)c5)C4)C3)n2)s1] | 1062 |
| 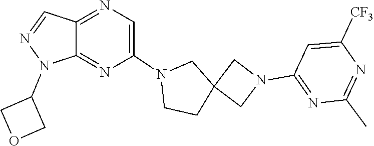 [SMILES: Cc1nc(N2CC3(CCN(c4cnc5cnn(C6COC6)c5n4)C3)C2)cc(C(F)(F)F)n1] | 1063 |
| 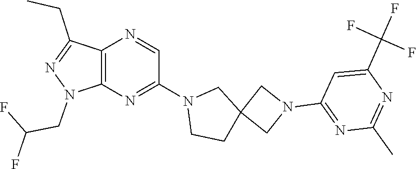 [SMILES: CCc1nn(CC(F)F)c2nc(N3CCC4(CN(c5cc(C(F)(F)F)nc(C)n5)C4)C3)cnc12] | 1064 |
| 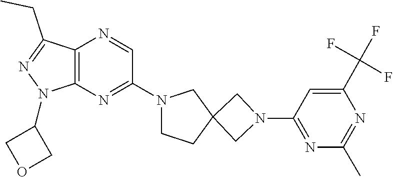 [SMILES: CCc1nn(C2COC2)c2nc(N3CCC4(CN(c5cc(C(F)(F)F)nc(C)n5)C4)C3)cnc12] | 1065 |
| 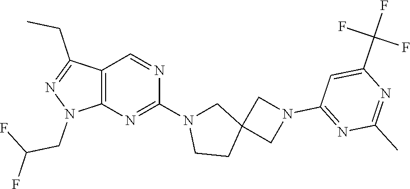 [SMILES: CCc1nn(CC(F)F)c2nc(N3CCC4(CN(c5cc(C(F)(F)F)nc(C)n5)C4)C3)ncc12] | 1066 |
| 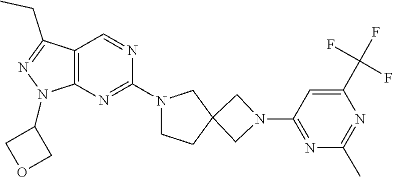 [SMILES: CCc1nn(C2COC2)c2nc(N3CCC4(CN(c5cc(C(F)(F)F)nc(C)n5)C4)C3)ncc12] | 1067 |
| 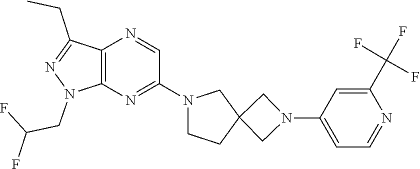 [SMILES: CCc1nn(CC(F)F)c2nc(N3CCC4(CN(c5ccnc(C(F)(F)F)c5)C4)C3)cnc12] | 1068 |
| 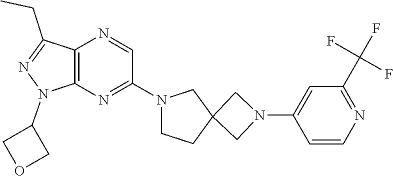 [SMILES: CCc1nn(C2COC2)c2nc(N3CCC4(CN(c5ccnc(C(F)(F)F)c5)C4)C3)cnc12] | 1069 |
| 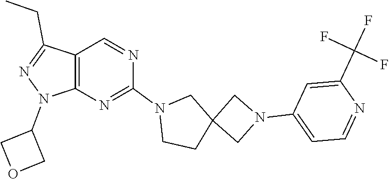 [SMILES: CCc1nn(C2COC2)c2nc(N3CCC4(CN(c5ccnc(C(F)(F)F)c5)C4)C3)ncc12] | 1070 |
| 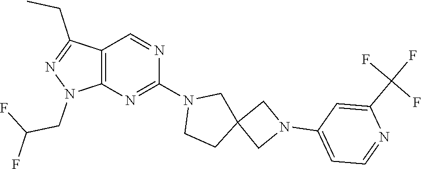 [SMILES: CCc1nn(CC(F)F)c2nc(N3CCC4(CN(c5ccnc(C(F)(F)F)c5)C4)C3)ncc12] | 1071 |
| 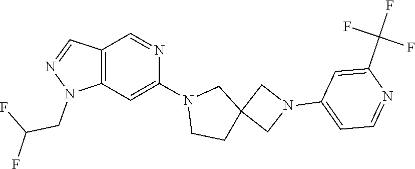 [SMILES: FC(F)Cn1ncc2cnc(N3CCC4(CN(c5ccnc(C(F)(F)F)c5)C4)C3)cc21] | 1072 |
| 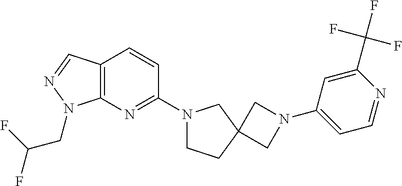 [SMILES: FC(F)Cn1ncc2ccc(N3CCC4(CN(c5ccnc(C(F)(F)F)c5)C4)C3)nc21] | 1073 |
| 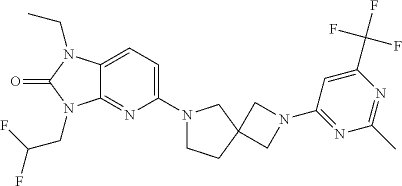 [SMILES: CCn1c(=O)n(CC(F)F)c2nc(N3CCC4(CN(c5cc(C(F)(F)F)nc(C)n5)C4)C3)ccc21] | 1074 |
| 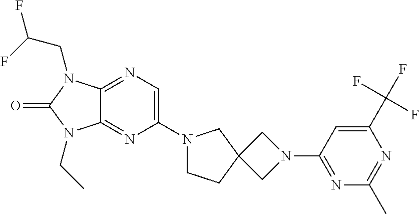 [SMILES: CCn1c(=O)n(CC(F)F)c2ncc(N3CCC4(CN(c5cc(C(F)(F)F)nc(C)n5)C4)C3)nc21] | 1075 |
| 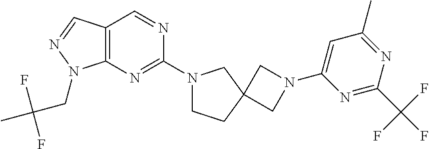 [SMILES: Cc1cc(N2CC3(CCN(c4ncc5cnn(CC(C)(F)F)c5n4)C3)C2)nc(C(F)(F)F)n1] | 1076 |
| 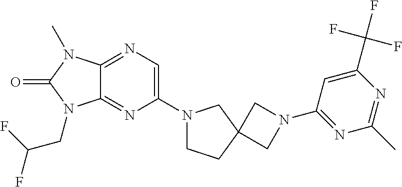 [SMILES: Cc1nc(N2CC3(CCN(c4cnc5c(n4)n(CC(F)F)c(=O)n5C)C3)C2)cc(C(F)(F)F)n1] | 1077 |
| 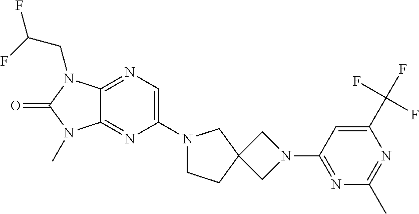 [SMILES: Cc1nc(N2CC3(CCN(c4cnc5c(n4)n(C)c(=O)n5CC(F)F)C3)C2)cc(C(F)(F)F)n1] | 1078 |
| 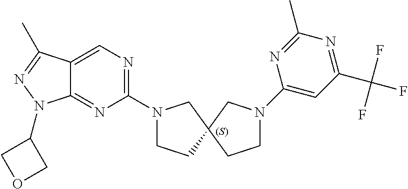 | 1079 |
| 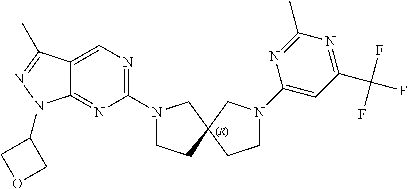 | 1080 |
| 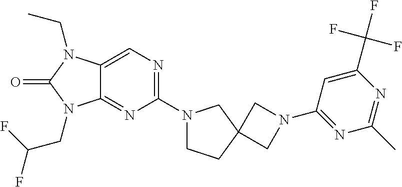 [SMILES: CCn1c(=O)n(CC(F)F)c2nc(N3CCC4(CN(c5cc(C(F)(F)F)nc(C)n5)C4)C3)ncc21] | 1081 |
| 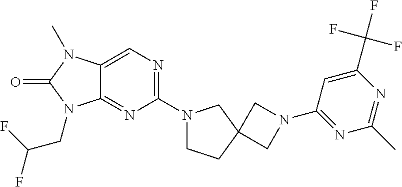 [SMILES: Cc1nc(N2CC3(CCN(c4ncc5c(n4)n(CC(F)F)c(=O)n5C)C3)C2)cc(C(F)(F)F)n1] | 1082 |
| 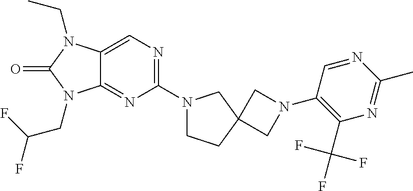 [SMILES: CCn1c(=O)n(CC(F)F)c2nc(N3CCC4(C3)CN(c3cnc(C)nc3C(F)(F)F)C4)ncc21] | 1083 |
| 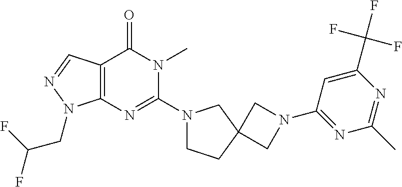 [SMILES: Cc1nc(N2CC3(CCN(c4nc5c(cnn5CC(F)F)c(=O)n4C)C3)C2)cc(C(F)(F)F)n1] | 1084 |
| 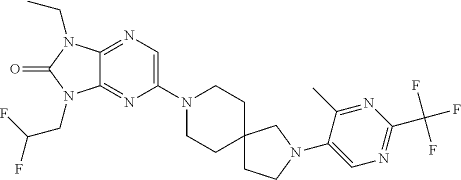 [SMILES: CCn1c(=O)n(CC(F)F)c2nc(N3CCC4(CC3)CCN(c3cnc(C(F)(F)F)nc3C)C4)cnc21] | 1085 |
| 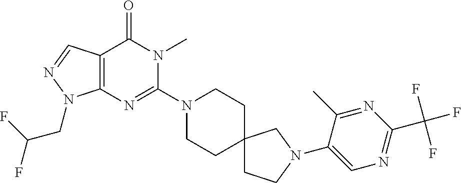 [SMILES: Cc1nc(C(F)(F)F)ncc1N1CCC2(CCN(c3nc4c(cnn4CC(F)F)c(=O)n3C)CC2)C1] | 1086 |
| 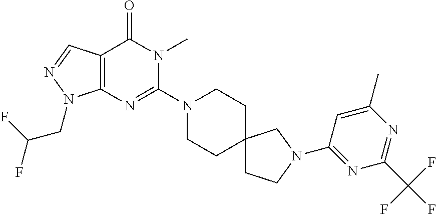 [SMILES: Cc1cc(N2CCC3(CCN(c4nc5c(cnn5CC(F)F)c(=O)n4C)CC3)C2)nc(C(F)(F)F)n1] | 1087 |
| 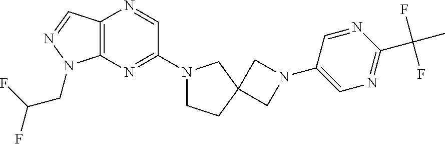 [SMILES: CC(F)(F)c1ncc(N2CC3(CCN(c4cnc5cnn(CC(F)F)c5n4)C3)C2)cn1] | 1088 |
| 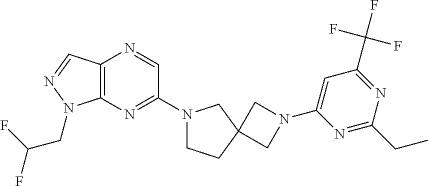 [SMILES: CCc1nc(N2CC3(CCN(c4cnc5cnn(CC(F)F)c5n4)C3)C2)cc(C(F)(F)F)n1] | 1089 |
| 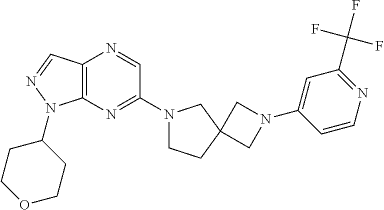 [SMILES: FC(F)(F)c1cc(N2CC3(CCN(c4cnc5cnn(C6CCOCC6)c5n4)C3)C2)ccn1] | 1090 |
| 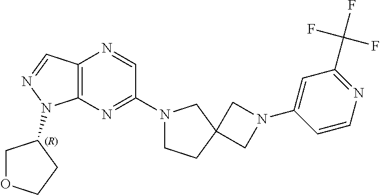 | 1091 |
| 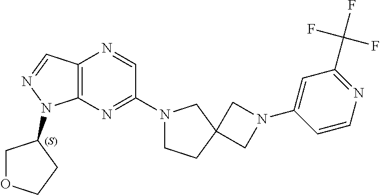 | 1092 |
| 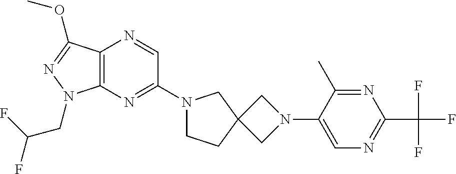 [SMILES: COc1nn(CC(F)F)c2nc(N3CCC4(C3)CN(c3cnc(C(F)(F)F)nc3C)C4)cnc12] | 1093 |
| 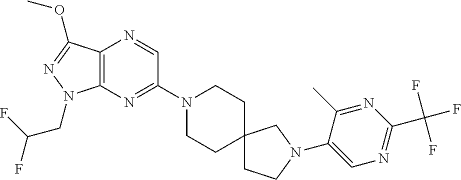 [SMILES: COc1nn(CC(F)F)c2nc(N3CCC4(CC3)CCN(c3cnc(C(F)(F)F)nc3C)C4)cnc12] | 1094 |
| 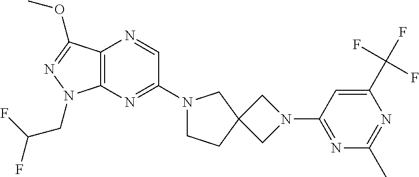 [SMILES: COc1nn(CC(F)F)c2nc(N3CCC4(CN(c5cc(C(F)(F)F)nc(C)n5)C4)C3)cnc12] | 1095 |
| 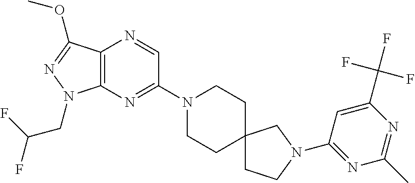 [SMILES: COc1nn(CC(F)F)c2nc(N3CCC4(CCN(c5cc(C(F)(F)F)nc(C)n5)C4)CC3)cnc12] | 1096 |
| 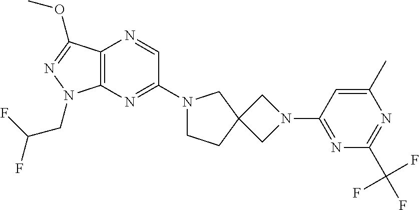 [SMILES: COc1nn(CC(F)F)c2nc(N3CCC4(CN(c5cc(C)nc(C(F)(F)F)n5)C4)C3)cnc12] | 1097 |
| 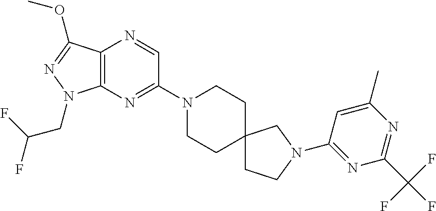 [SMILES: COc1nn(CC(F)F)c2nc(N3CCC4(CCN(c5cc(C)nc(C(F)(F)F)n5)C4)CC3)cnc12] | 1098 |
| 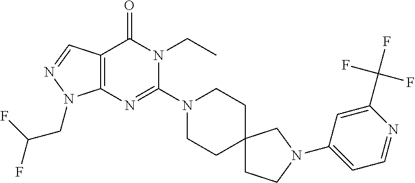 [SMILES: CCn1c(N2CCC3(CCN(c4ccnc(C(F)(F)F)c4)C3)CC2)nc2c(cnn2CC(F)F)c1=O] | 1099 |
| 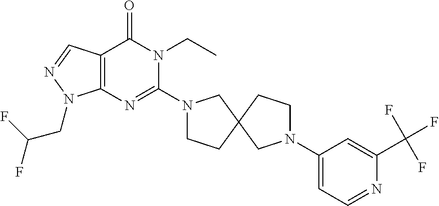 [SMILES: CCn1c(N2CCC3(CCN(c4ccnc(C(F)(F)F)c4)C3)C2)nc2c(cnn2CC(F)F)c1=O] | 1100 |
| 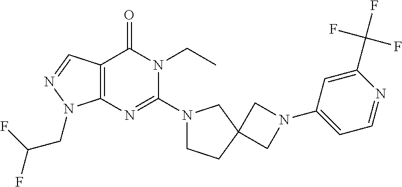 [SMILES: CCn1c(N2CCC3(CN(c4ccnc(C(F)(F)F)c4)C3)C2)nc2c(cnn2CC(F)F)c1=O] | 1101 |
| 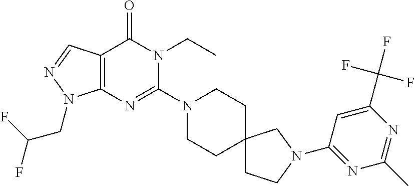 [SMILES: CCn1c(N2CCC3(CCN(c4cc(C(F)(F)F)nc(C)n4)C3)CC2)nc2c(cnn2CC(F)F)c1=O] | 1102 |
| 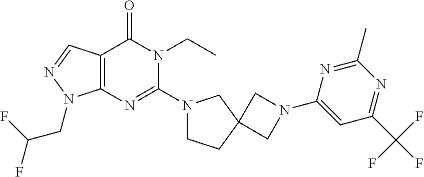 [SMILES: CCn1c(N2CCC3(CN(c4cc(C(F)(F)F)nc(C)n4)C3)C2)nc2c(cnn2CC(F)F)c1=O] | 1103 |
| 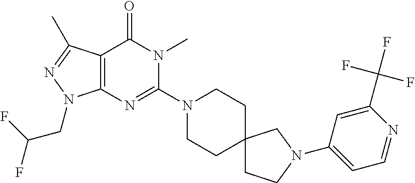 [SMILES: Cc1nn(CC(F)F)c2nc(N3CCC4(CCN(c5ccnc(C(F)(F)F)c5)C4)CC3)n(C)c(=O)c12] | 1104 |
| 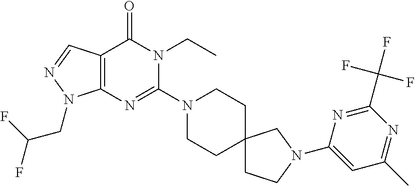 [SMILES: CCn1c(N2CCC3(CCN(c4cc(C)nc(C(F)(F)F)n4)C3)CC2)nc2c(cnn2CC(F)F)c1=O] | 1105 |
| 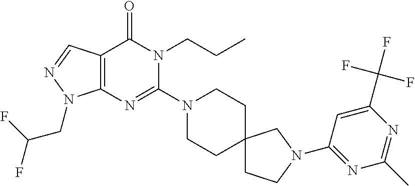 [SMILES: CCCn1c(N2CCC3(CCN(c4cc(C(F)(F)F)nc(C)n4)C3)CC2)nc2c(cnn2CC(F)F)c1=O] | 1106 |
| 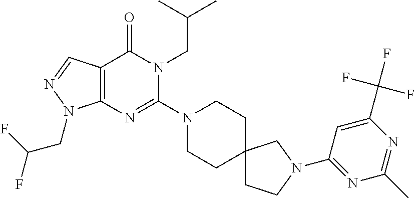 [SMILES: Cc1nc(N2CCC3(CCN(c4nc5c(cnn5CC(F)F)c(=O)n4CC(C)C)CC3)C2)cc(C(F)(F)F)n1] | 1107 |
| 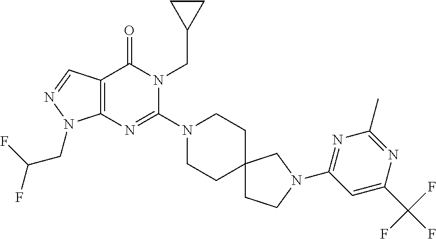 [SMILES: Cc1nc(N2CCC3(CCN(c4nc5c(cnn5CC(F)F)c(=O)n4CC4CC4)CC3)C2)cc(C(F)(F)F)n1] | 1108 |
| 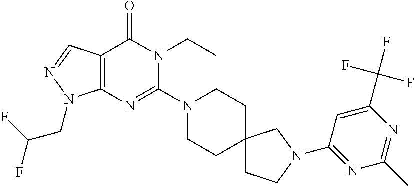 [SMILES: CCn1c(N2CCC3(CCN(c4cc(C(F)(F)F)nc(C)n4)C3)CC2)nc2c(cnn2CC(F)F)c1=O] | 1109 |
| 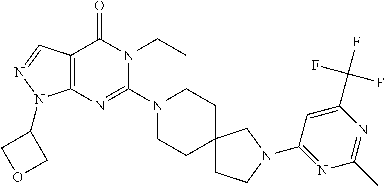 [SMILES: CCn1c(N2CCC3(CCN(c4cc(C(F)(F)F)nc(C)n4)C3)CC2)nc2c(cnn2C2COC2)c1=O] | 1110 |
| 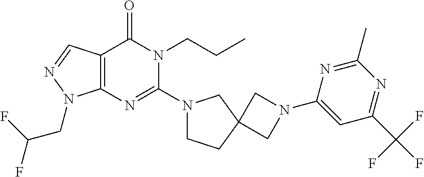 [SMILES: CCCn1c(N2CCC3(CN(c4cc(C(F)(F)F)nc(C)n4)C3)C2)nc2c(cnn2CC(F)F)c1=O] | 1111 |
| 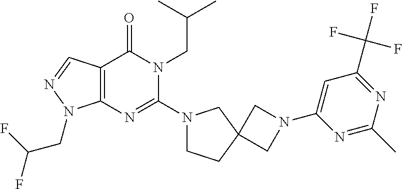 [SMILES: Cc1nc(N2CC3(CCN(c4nc5c(cnn5CC(F)F)c(=O)n4CC(C)C)C3)C2)cc(C(F)(F)F)n1] | 1112 |
| 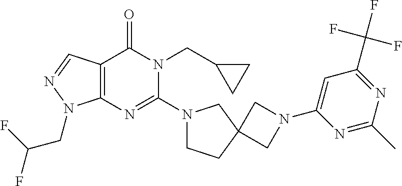 [SMILES: Cc1nc(N2CC3(CCN(c4nc5c(cnn5CC(F)F)c(=O)n4CC4CC4)C3)C2)cc(C(F)(F)F)n1] | 1113 |
| 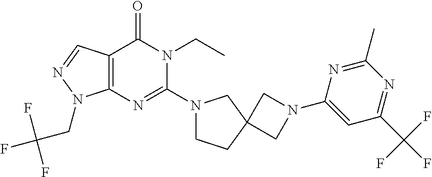 [SMILES: CCn1c(N2CCC3(CN(c4cc(C(F)(F)F)nc(C)n4)C3)C2)nc2c(cnn2CC(F)(F)F)c1=O] | 1114 |
| 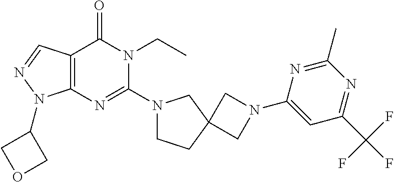 [SMILES: CCn1c(N2CCC3(CN(c4cc(C(F)(F)F)nc(C)n4)C3)C2)nc2c(cnn2C2COC2)c1=O] | 1115 |
| 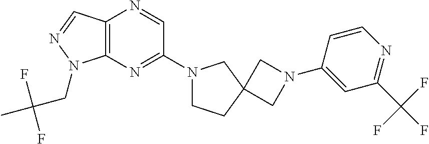 [SMILES: CC(F)(F)Cn1ncc2ncc(N3CCC4(CN(c5ccnc(C(F)(F)F)c5)C4)C3)nc21] | 1116 |
| 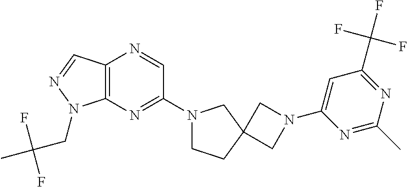 [SMILES: Cc1nc(N2CC3(CCN(c4cnc5cnn(CC(C)(F)F)c5n4)C3)C2)cc(C(F)(F)F)n1] | 1117 |
| 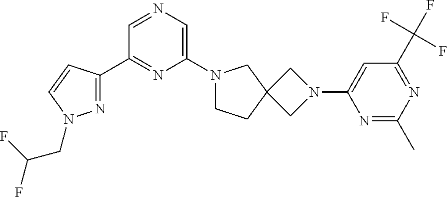 [SMILES: Cc1nc(N2CC3(CCN(c4cncc(-c5ccn(CC(F)F)n5)n4)C3)C2)cc(C(F)(F)F)n1] | 1118 |
| 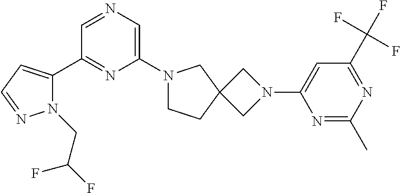 [SMILES: Cc1nc(N2CC3(CCN(c4cncc(-c5ccnn5CC(F)F)n4)C3)C2)cc(C(F)(F)F)n1] | 1119 |
| 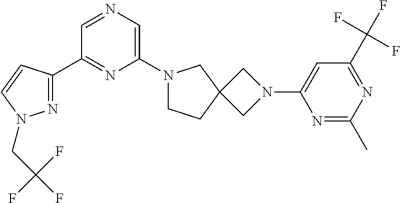 [SMILES: Cc1nc(N2CC3(CCN(c4cncc(-c5ccn(CC(F)(F)F)n5)n4)C3)C2)cc(C(F)(F)F)n1] | 1120 |
| 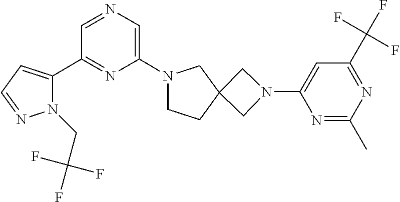 [SMILES: Cc1nc(N2CC3(CCN(c4cncc(-c5ccnn5CC(F)(F)F)n4)C3)C2)cc(C(F)(F)F)n1] | 1121 |
| 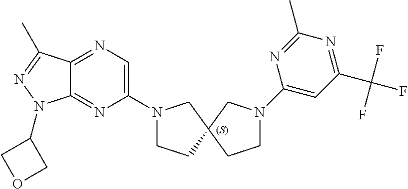 | 1122 |
| 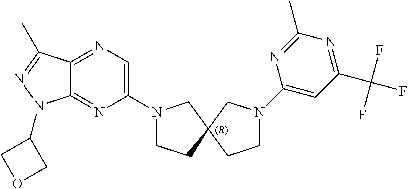 | 1123 |
| 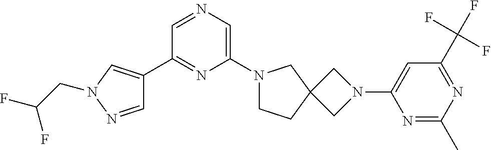 [SMILES: Cc1nc(N2CC3(CCN(c4cncc(-c5cnn(CC(F)F)c5)n4)C3)C2)cc(C(F)(F)F)n1] | 1124 |
| 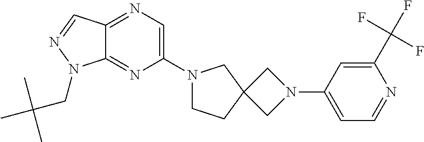 [SMILES: CC(C)(C)Cn1ncc2ncc(N3CCC4(CN(c5ccnc(C(F)(F)F)c5)C4)C3)nc21] | 1125 |
| 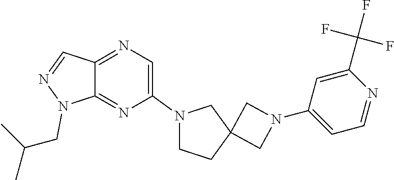 [SMILES: CC(C)Cn1ncc2ncc(N3CCC4(CN(c5ccnc(C(F)(F)F)c5)C4)C3)nc21] | 1126 |
| 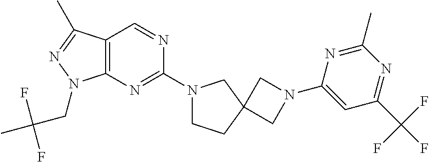 [SMILES: Cc1nc(N2CC3(CCN(c4ncc5c(C)nn(CC(C)(F)F)c5n4)C3)C2)cc(C(F)(F)F)n1] | 1127 |
| 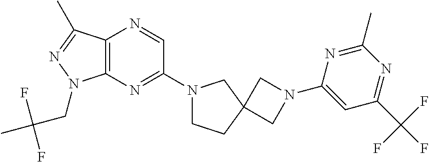 [SMILES: Cc1nc(N2CC3(CCN(c4cnc5c(C)nn(CC(C)(F)F)c5n4)C3)C2)cc(C(F)(F)F)n1] | 1128 |
| 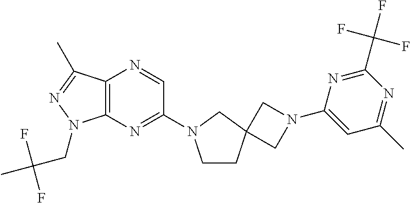 [SMILES: Cc1cc(N2CC3(CCN(c4cnc5c(C)nn(CC(C)(F)F)c5n4)C3)C2)nc(C(F)(F)F)n1] | 1129 |
| 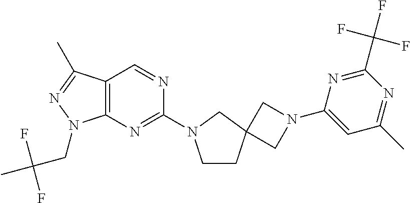 [SMILES: Cc1cc(N2CC3(CCN(c4ncc5c(C)nn(CC(C)(F)F)c5n4)C3)C2)nc(C(F)(F)F)n1] | 1130 |
| 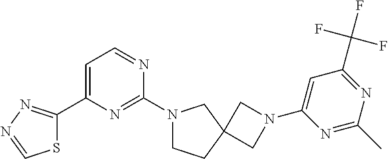 [SMILES: Cc1nc(N2CC3(CCN(c4nccc(-c5nncs5)n4)C3)C2)cc(C(F)(F)F)n1] | 1131 |
| 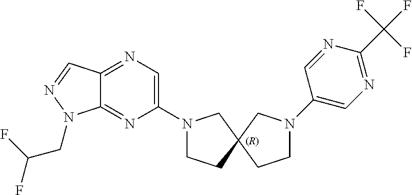 | 1132 |
| 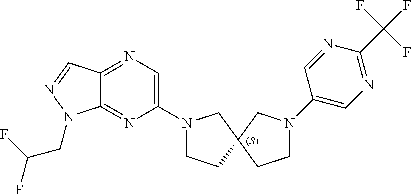 | 1133 |
| 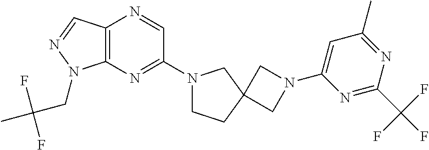 [SMILES: Cc1cc(N2CC3(CCN(c4cnc5cnn(CC(C)(F)F)c5n4)C3)C2)nc(C(F)(F)F)n1] | 1134 |
| 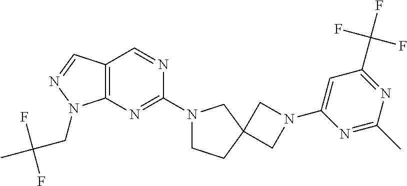 [SMILES: Cc1nc(N2CC3(CCN(c4ncc5cnn(CC(C)(F)F)c5n4)C3)C2)cc(C(F)(F)F)n1] | 1135 |
| 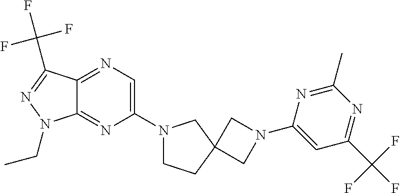 [SMILES: CCn1nc(C(F)(F)F)c2ncc(N3CCC4(CN(c5cc(C(F)(F)F)nc(C)n5)C4)C3)nc21] | 1136 |
| 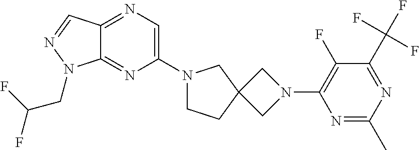 [SMILES: Cc1nc(N2CC3(CCN(c4cnc5cnn(CC(F)F)c5n4)C3)C2)c(F)c(C(F)(F)F)n1] | 1137 |
| 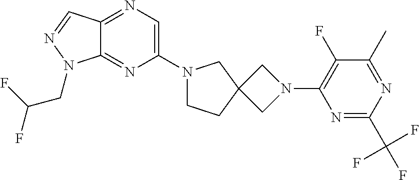 [SMILES: Cc1nc(C(F)(F)F)nc(N2CC3(CCN(c4cnc5cnn(CC(F)F)c5n4)C3)C2)c1F] | 1138 |
| 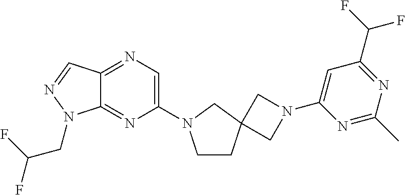 [SMILES: Cc1nc(C(F)F)cc(N2CC3(CCN(c4cnc5cnn(CC(F)F)c5n4)C3)C2)n1] | 1139 |
| 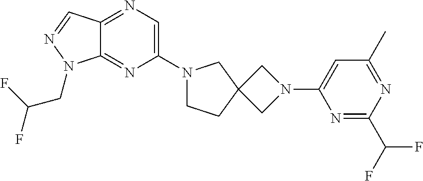 [SMILES: Cc1cc(N2CC3(CCN(c4cnc5cnn(CC(F)F)c5n4)C3)C2)nc(C(F)F)n1] | 1140 |
| 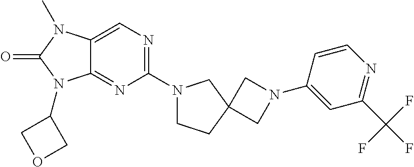 [SMILES: Cn1c(=O)n(C2COC2)c2nc(N3CCC4(CN(c5ccnc(C(F)(F)F)c5)C4)C3)ncc21] | 1141 |
| 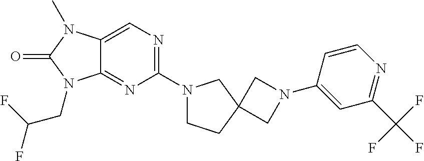 [SMILES: Cn1c(=O)n(CC(F)F)c2nc(N3CCC4(CN(c5ccnc(C(F)(F)F)c5)C4)C3)ncc21] | 1142 |
| 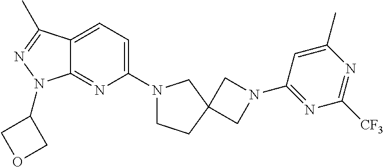 [SMILES: Cc1cc(N2CC3(CCN(c4ccc5c(C)nn(C6COC6)c5n4)C3)C2)nc(C(F)(F)F)n1] | 1143 |
| 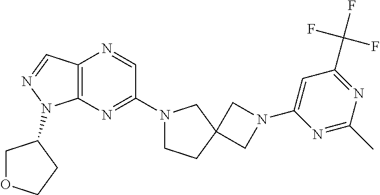 [SMILES: Cc1nc(N2CC3(CCN(c4cnc5cnn([C@@H]6CCOC6)c5n4)C3)C2)cc(C(F)(F)F)n1] | 1144 |
| 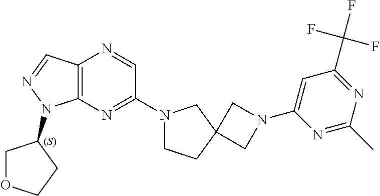 | 1145 |
| 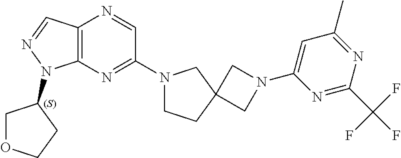 | 1146 |
| 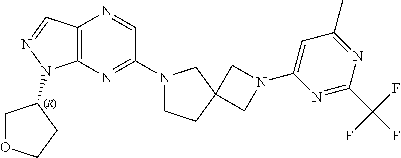 | 1147 |

In certain embodiments, the provided compounds (e.g., compounds of Formula (I)), activate GCase with an EC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

### Pharmaceutical Compositions, Kits, and Administration

The present disclosure provides pharmaceutical compositions comprising a disclosed compound (e.g., a compound of Formula (I)), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound of Formula (I) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a disease or disorder in a subject in need thereof. In certain embodiments, the effective amount is an amount effective for treating a neurological disease or disorder in a subject in need thereof. In certain embodiments, the effective amount is an amount effective for preventing a neurological disease or disorder in a subject in need thereof.

In certain embodiments, the effective amount is an amount effective for reducing the risk of developing a disease (e.g., neurological disease or disorder) in a subject in need thereof.

In certain embodiments, the effective amount is an amount effective for increasing the activity of GCase in a subject, tissue, biological sample, or cell.

In certain embodiments, the subject being treated or administered a compound described herein is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for increasing the activity of GCase by at least about 10%, at least about 20%, at least about 30%, at least about 40%, at least about 50%, at least about 60%, at least about 70%, at least about 80%, at least about 90%, at least about 95%, at least about 100%, at least about 150%, at least about 200%, at least about 250%, at least about 300%, at least about 400%, at least about 500%, or at least about 1000%. In certain embodiments, the effective amount is an amount effective for increasing the activity of GCase by a range between a percentage described in this paragraph and another percentage described in this paragraph, inclusive.

The present disclosure provides pharmaceutical compositions comprising a compound that interacts with (e.g., activates) GCase for use in treating a GCase-related disease or disorder in a subject in need thereof. The present disclosure provides pharmaceutical compositions comprising a compound that interacts with (e.g., activates) GCase for use in treating a disease or disorder associated with aberrant activity of GCase in a subject in need thereof. The present disclosure provides pharmaceutical compositions comprising a compound that interacts with (e.g., activates) GCase for use in treating a disease or disorder associated with mutated GCase in a subject in need thereof.

In certain embodiments, the composition is for use in treating a disease or disorder. In certain embodiments, the composition is for use in treating a neurological disease or disorder. In certain embodiments, the composition is for use in treating Gaucher's disease or Parkinson's disease. In certain embodiments, the composition is for use in treating Gaucher's disease. In certain embodiments, the composition is for use in treating Parkinson's disease.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological disease or disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of the present disclosure can be administered to humans and other animals orally, parenterally, intracisternally, intraperitoneally, topically, bucally, or the like, depending on the disease or condition being treated.

In certain embodiments, a pharmaceutical composition comprising a compound of Formula (I) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., neurological disease or disorder) in a subject in need thereof. In certain embodiments, the kits are useful for preventing a disease (e.g., neurological disease or disorder) in a subject in need thereof. In certain embodiments, the kits are useful for reducing the risk of developing a disease (e.g., neurological disease or disorder) in a subject in need thereof. In certain embodiments, the kits are useful for increasing the activity of GCase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., neurological disease or disorder) in a subject in need thereof. In certain embodiments, the kits and instructions provide for preventing a disease (e.g., neurological disease or disorder) in a subject in need thereof. In certain embodiments, the kits and instructions provide for reducing the risk of developing a disease (e.g., neurological disease or disorder) in a subject in need thereof. In certain embodiments, the kits and instructions provide for increasing the activity of GCase in a subject or cell. A kit described herein may include one or more additional pharmaceutical agents described herein as a separate composition.

### Methods of Treatment

The present disclosure provides methods for treating a disease or disorder in a subject in need thereof. In certain embodiments, the present disclosure provides methods for treating a disease or disorder associated with GCase activity. In certain embodiments, the application provides a method of treating a neurological disease or disorder. In certain embodiments, the application provides a method of treating Gaucher's disease or Parkinson's disease. In certain embodiments, the application provides a method of treating Gaucher's disease. In certain embodiments, the application provides a method of treating Parkinson's disease.

The present disclosure provides a method of activating GCase. The present disclosure provides a method of increasing the activity of GCase. In certain embodiments, the application provides a method of activating GCase (e.g., increasing the activity of GCase) in vitro. In certain embodiments, the application provides a method of activating GCase (e.g., increasing the activity of GCase) in vivo. In certain embodiments, the application provides a method of increasing the activity of GCase in a cell. In certain embodiments, the application provides a method of increasing the activity of GCase in a human cell.

In certain embodiments, the methods comprise administering to a subject in need thereof (e.g., a subject with a neurological disease or disorder) a compound that interacts with GCase, for example, a compound that is a modulator of GCase (e.g., an activator of GCase), a binder of GCase, or a compound that modifies GCase. In certain embodiments, the methods comprise administering a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof.

Another object of the present disclosure is the use of a compound as described herein (e.g., of any formulae herein) in the manufacture of a medicament for use in the treatment of a disorder or disease described herein. Another object of the present disclosure is the use of a compound as described herein (e.g., of any formulae herein) for use in the treatment of a disorder or disease described herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Synthetic Methods

Compounds of Formula (I) were prepared following the synthetic schemes and procedures described in detail below. The examples described in this application are offered to illustrate the compounds, pharmaceutical compositions, and methods provided herein and are not to be construed in any way as limiting their scope. Compounds of the disclosure that are not explicitly described in the following procedures may be prepared by analogous methods. Those having ordinary skill in the art would understand how to make such compounds from the disclosure provided herein and by means known in the art of organic synthesis. For example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof are representative and instructive. Methods for optimizing reaction conditions, if necessary minimizing competing by products, are known in the art. Embodiments of this disclosure include methods of synthesizing compounds delineated herein using any of the compounds, reactants, and/or processes delineated herein.

In the naming of certain compounds, the word “assumed” is written immediately after the name of the compound. This is meant to designate that the assigned stereochemical centers of that compound are assumed to be the orientation named in the compound name.

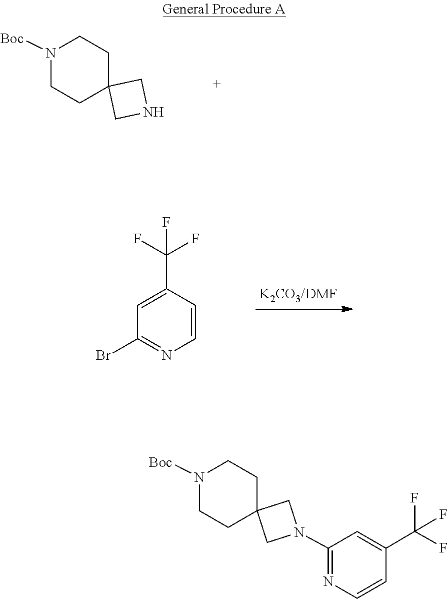 [SMILES: CC(C)(C)OC(=O)N1CCC2(CC1)CNC2 ; FC(F)(F)c1ccnc(Br)c1 ; CC(C)(C)OC(=O)N1CCC2(CC1)CN(c1cc(C(F)(F)F)ccn1)C2]

tert-Butyl 2-[4-(trifluoromethyl)pyridin-2-yl]-2,7-diazaspiro[3.5]nonane-7-carboxylate: A mixture of tert-butyl 2,7-diazaspiro[3.5]nonane-7-carboxylate (100 mg, 442 μmol, 1.0 equiv.), 2-bromo-4-(trifluoromethyl)pyridine (99.9 mg, 442 μmol, 1.0 equiv.), and K₂CO₃ (183 mg, 1.33 mmol, 3.0 equiv.) in DMF (1 mL) was heated at 75° C. overnight. The reaction was monitored by LCMS. The mixture was diluted with water (5 mL), and extracted with EtOAc (5 mL×2). The organic layer was combined, washed with brine, dried, evaporated, and purified with a silico gel column, eluted with gradient of Hexane/EtOAc. The fractions were collected, and concentrated to give tert-butyl 2-[4-(trifluoromethyl)pyridin-2-yl]-2,7-diazaspiro[3.5]nonane-7-carboxylate (140 mg, 85%) as a colorless oil.

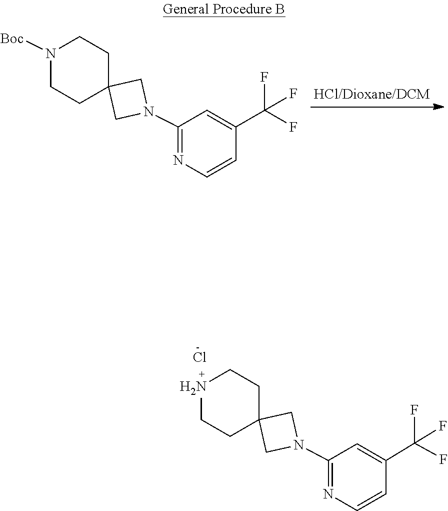 [SMILES: CC(C)(C)OC(=O)N1CCC2(CC1)CN(c1cc(C(F)(F)F)ccn1)C2 ; Cl ; FC(F)(F)c1ccnc(N2CC3(CCNCC3)C2)c1]

2-[4-(Trifluoromethyl)pyridin-2-yl]-2,7-diazaspiro[3.5]nonane hydrochloride: To a stirred solution of tert-butyl 2-[4-(trifluoromethyl)pyridin-2-yl]-2,7-diazaspiro[3.5]nonane-7-carboxylate (140 mg, 377 μmol, 1.0 equiv.) in DCM (2.5 mL) was added HCl (gas) in dioxane (4M, 1.88 mL, 20 equiv.) in a ice bath. The mixture was allowed to stir at room temperature for 2 h. After removing the solvent, the crude product 2-[4-(trifluoromethyl)pyridin-2-yl]-2,7-diazaspiro[3.5]nonane hydrochloride (102 mg) was directly used in next step without further purification.

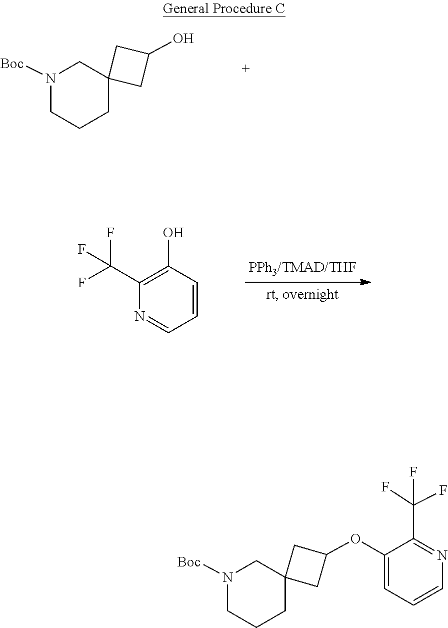 [SMILES: CC(C)(C)OC(=O)N1CCCC2(CC(O)C2)C1 ; Oc1cccnc1C(F)(F)F ; CC(C)(C)OC(=O)N1CCCC2(CC(Oc3cccnc3C(F)(F)F)C2)C1]

tert-Butyl 2-{[2-(trifluoromethyl)pyridin-3-yl]oxy}-6-azaspiro[3.5]nonane-6-carboxylate: 2-(Trifluoromethyl)pyridin-3-ol (67.6 mg, 414 μmol) and triphenylphosphane (174 mg, 663 μmol, 1.6 equiv.) were added to a solution of tert-butyl 2-hydroxy-6-azaspiro[3.5]nonane-6-carboxylate (100 mg, 414 μmol, 1.0 equiv.) in anhydrous THF (10.0 mL) under an Argon atmosphere. The reaction mixture was cooled to 0° C. and 1,1′-Azobis(N,N-dimethylformamide) (114 mg, 663 μmol, 1.6 equiv.) was added. The solution was allowed to warm to room temperature and stirred overnight. The reaction mixture was concentrated under reduced pressure then stirred with 10 mL Hexane:EtOAc (3:1) mixture, and filtered. The filtrate was concentrated. The residue was purified on column chromatography to give tert-butyl 2-{[2-(trifluoromethyl)pyridin-3-yl]oxy}-6-azaspiro[3.5]nonane-6-carboxylate (148 mg, 92%) as a light-yellow oil.

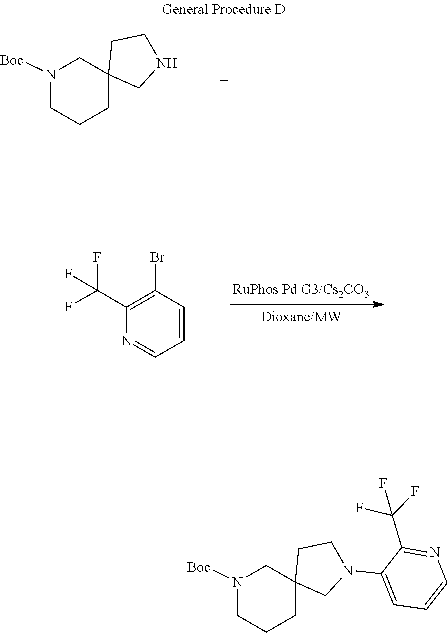 [SMILES: CC(C)(C)OC(=O)N1CCCC2(CCNC2)C1 ; FC(F)(F)c1ncccc1Br ; CC(C)(C)OC(=O)N1CCCC2(CCN(c3cccnc3C(F)(F)F)C2)C1]

tert-Butyl 2-[2-(trifluoromethyl)pyridin-3-yl]-2,7-diazaspiro[4.5]decane-7-carboxylate: A mixture of tert-butyl 2,7-diazaspiro[4.5]decane-7-carboxylate (198 mg, 825 μmol, 1.0 equiv), 3-bromo-2-(trifluoromethyl)pyridine (280 mg, 825 μmol, 1.0 equiv.), RuPhos Pd G3 (69 mg, 82.5 μmol, 0.1 equiv.), and Cs₂CO₃ (538 mg, 1.65 mmol, 2.0 equiv.) in 1,4-dioxane (3 mL) was heated in a microwave reactor at 150° C. for 20 min. The reaction was monitored by LCMS. The mixture was filtered through Celite, and washed with EtOAc (10 mL). The organic layer was combined, evaporated, and purified with a silico gel column, eluted with gradient of Hexane/EtOAc. The fractions were collected, and concentrated to give tert-butyl 2-[2-(trifluoromethyl)pyridin-3-yl]-2,7-diazaspiro[4.5]decane-7-carboxylate (243 mg, 61%) as a yellow oil.

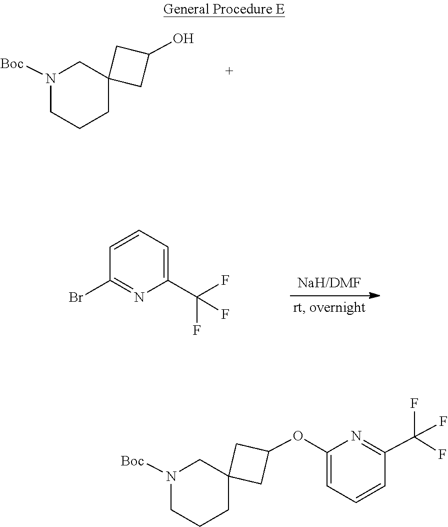 [SMILES: CC(C)(C)OC(=O)N1CCCC2(CC(O)C2)C1 ; FC(F)(F)c1cccc(Br)n1 ; CC(C)(C)OC(=O)N1CCCC2(CC(Oc3cccc(C(F)(F)F)n3)C2)C1]

To a stirred solution of tert-butyl 2-hydroxy-6-azaspiro[3.5]nonane-6-carboxylate (100 mg, 414 μmol, in DMF (3.00 mL) was added NaH (60% w/w, 18.2 mg, 456 μmol, 1.1 equiv.) at 0° C., and allowed to warm up to room temperature and stirred for 15 min. 2-bromo-6-(trifluoromethyl)pyridine (93.6 mg, 414 μmol, 1 equiv.) was added to the mixture, and the mixture was heated at rt for 20 h. The reaction was monitored by LCMS. The mixture was diluted with water, and extracted with EtOAc (20 mL×2). The organic layer was washed with brine, dried, filtered, evaporated, and purified by Combi-Flash to give tert-butyl 2-{[6-(trifluoromethyl)pyridin-2-yl]oxy}-6-azaspiro[3.5]nonane-6-carboxylate (138 mg, 86%) as a colorless oil.

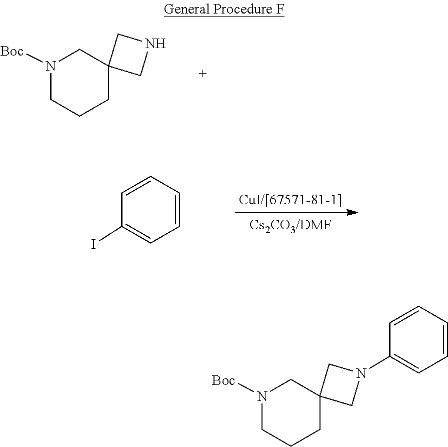 [SMILES: CC(C)(C)OC(=O)N1CCCC2(CNC2)C1 ; Ic1ccccc1 ; CC(C)(C)OC(=O)N1CCCC2(C1)CN(c1ccccc1)C2]

To a stirred solution of tert-butyl 2,6-diazaspiro[3.5]nonane-6-carboxylate hydrochloride (500 mg, 1.9 mmol, 1 equiv.) and iodobenzene (776 mg, 1.9 mmom, 1 equiv.) in DMF (2 mL) were added caesium carbonate (1.86 g, 5.71 mmol, 3 equiv.), iodocopper (+1) (36.2 mmol, 0.19 mmol, 1 equiv.), rel-(1R,2R)—N1,N2-dimethylcyclohexane-1,2-diamine (27 mg, 0.19 mmol, 1 equiv.). The resulting mixture was heated at 120° C. for 30 h under an Argon atmosphere. The mixture was cooled to rt, filtered through Celite, and washed with EtOAc. The filtrate was concentrated, and purified with Combi-flash to give tert-butyl 2-phenyl-2,6-diazaspiro[3.5]nonane-6-carboxylate (200 mg, 35%) as a white solid.

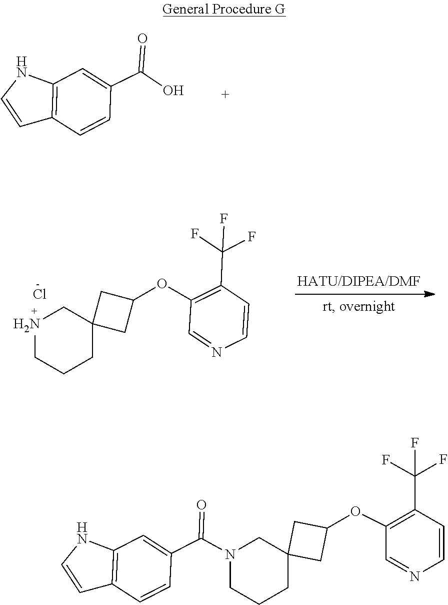 [SMILES: O=C(O)c1ccc2cc[nH]c2c1 ; FC(F)(F)c1ccncc1OC1CC2(CCCNC2)C1 ; Cl ; O=C(c1ccc2cc[nH]c2c1)N1CCCC2(CC(Oc3cnccc3C(F)(F)F)C2)C1]

A mixture of 1H-indole-6-carboxylic acid (12 mg, 77.5 μmo, 1 equiv.) and HATU (32.4 mg, 85.2 μmol, 1.1 equiv.) were added to DMF (1.00 mL) followed by DIPEA (40.5 μL, 232 μmol, 3 equiv.) and 2-{[4-(trifluoromethyl)pyridin-3-yl]oxy}-6-azaspiro[3.5]nonane hydrochloride (25 mg, 77.5 μmol, 1 equiv.) at room temperature. The mixture was stirred at room temperature for 16 h. The reaction was monitored by LCMS. The mixture was diluted with water (15 mL), and extracted with EtOAc (15 mL×2). The organic layer was combined, washed with brine, dried, evaporated, and purified with Combi-flash (4 g silico gel column), eluted with Hex:EtOAc. The fractions were collected, and concentrated to afford the product 6-(1H-indole-6-carbonyl)-2-{[4-(trifluoromethyl)pyridin-3-yl]oxy}-6-azaspiro[3.5]nonane (25 mg, 75%, two isomers in a ratio of 1:5) as a white solid.

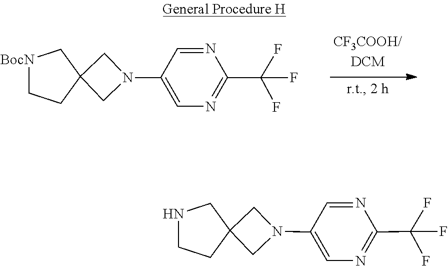 [SMILES: CC(C)(C)OC(=O)N1CCC2(C1)CN(c1cnc(C(F)(F)F)nc1)C2 ; FC(F)(F)c1ncc(N2CC3(CCNC3)C2)cn1]

2-(2-(trifluoromethyl)pyrimidin-5-yl)-2,6-diazaspiro[3.4]octane: To a stirred solution of tert-butyl 2-(2-(trifluoromethyl)pyrimidin-5-yl)-2,6-diazaspiro[3.4]octane-6-carboxylate (5 g, 13.9 mmol, 1 equiv) in DCM (50 mL) was added TFA (50 mL) dropwise at 0° C. The resulting mixture was stirred for additional 2 h at room temperature. The resulting mixture was concentrated under reduced pressure. The crude product 2-(2-(trifluoromethyl)pyrimidin-5-yl)-2,6-diazaspiro[3.4]octane (10 g, crude) was used in the next step directly without further purification.

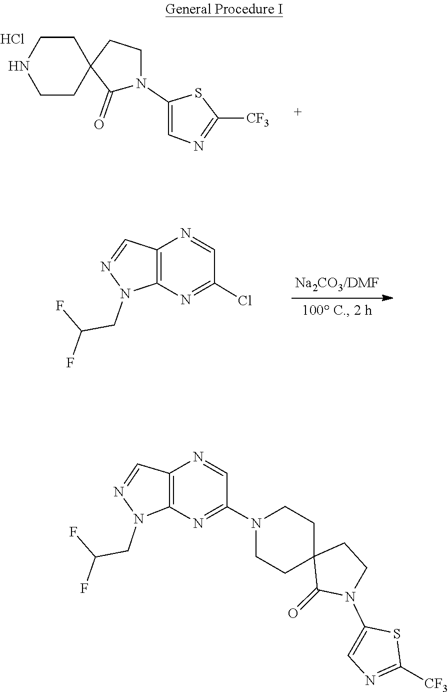

8-(1-(2,2-difluoroethyl)-1H-pyrazolo[3,4-b]pyrazin-6-yl)-2-(2-(trifluoromethyl)thiazol-5-yl)-2,8-diazaspiro[4.5]decan-1-one: To a stirred solution of 2-(2-(trifluoromethyl)thiazol-5-yl)-2,8-diaza-spiro[4.5]decan-1-one hydrochloride (93.8 mg, 0.275 mmol, 1.0 equiv) and 6-chloro-1-(2,2-difluoroethyl)-1H-pyrazolo[3,4-b]pyrazine (60 mg, 0.275 mmol, 1.0 equiv) in DMF (1.00 mL) was added Na₂CO₃ (87.4 mg, 0.825 mmol, 3.0 equiv). The resulting mixture was stirred for 2 h at 100° C. Desired product could be detected by LCMS. The mixture was diluted with EtOAc (15 mL), washed with water and brine, dried, evaporated, and purified with Combi-flash (40 g silico gel column), eluted with Hex:EtOAc. The fractions were collected, and concentrated to give 8-(1-(2,2-difluoroethyl)-1H-pyrazolo[3,4-b]pyrazin-6-yl)-2-(2-(trifluoromethyl)thiazol-5-yl)-2,8-diazaspiro[4.5]decan-1-one (57.7 mg, 43.0%) as a white solid.

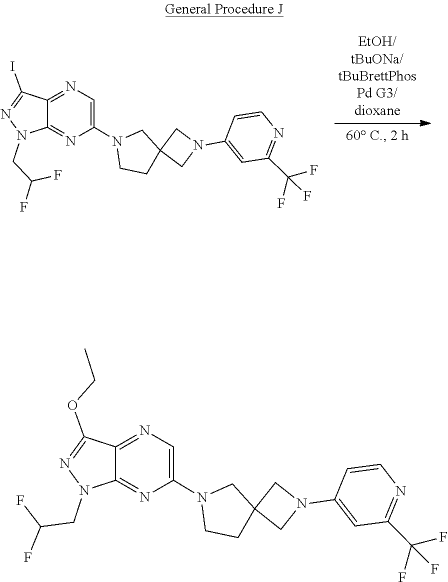 [SMILES: FC(F)Cn1nc(I)c2ncc(N3CCC4(CN(c5ccnc(C(F)(F)F)c5)C4)C3)nc21 ; CCOc1nn(CC(F)F)c2nc(N3CCC4(CN(c5ccnc(C(F)(F)F)c5)C4)C3)cnc12]

1-(2,2-difluoroethyl)-3-ethoxy-6-(2-(2-(trifluoromethyl)pyridin-4-yl)-2,6-diazaspiro[3.4]octan-6-yl)-1H-pyrazolo[3,4-b]pyrazine: To the solution of 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine (100 mg, 0.177 mmol, 1 equiv) and EtOH (14.6 mg, 0.318 mmol, 2 equiv) in dioxane (1 mL) were added tBuBrettPhos Pd G3 (15.1 mg, 0.018 mmol, 0.1 equiv) and tBuONa (34.00 mg, 0.354 mmol, 2 equiv) under N₂ atmosphere. The result mixture was heated to 60° C. and stirred for 2 h. Desired product could be detected by LCMS. The reaction mixture was diluted by EtOAc (20 mL), washed by water (2×15 mL) and brine (1×15 mL), dried over anhydrous sodium sulfate. After filtration, the filtrate was concentrated. The residue was purified with Combi-flash (40 g silico gel column), eluted with Hex:EtOAc. The fractions were collected, and concentrated to give 1-(2,2-difluoroethyl)-3-ethoxy-6-(2-(2-(trifluoromethyl)pyridin-4-yl)-2,6-diazaspiro[3.4]octan-6-yl)-1H-pyrazolo[3,4-b]pyrazine (18.1 mg, 21.2%) as a white solid.

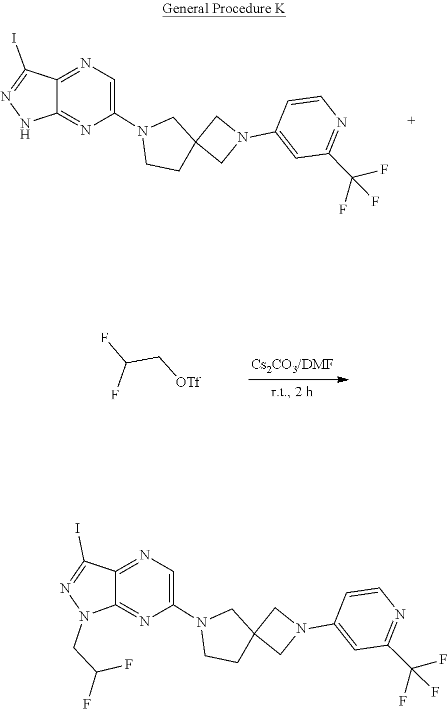

1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine: To a stirred mixture of 3-iodo-6-(2-(2-(trifluoromethyl)pyridin-4-yl)-2,6-diazaspiro[3.4]octan-6-yl)-1H-pyrazolo[3,4-b]pyrazine (240 mg, 0.479 mmol, 1 equiv) and Cs₂CO₃ (312 mg, 0.958 mmol, 2 equiv) in DMF (2.5 mL) was added 2,2-difluoroethyl trifluoromethanesulfonate (112 mg, 0.527 mmol, 1.1 equiv) at 0° C. The resulting mixture was stirred for 1 h at room temperature. Desired product could be detected by LCMS. The mixture was diluted with EtOAc (15 mL), washed with water and brine, dried, evaporated, and purified with Combi-flash (40 g silico gel column), eluted with Hex:EtOAc. The fractions were collected, and concentrated to give 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine to afford (100 mg, 36.9%) as a white solid.

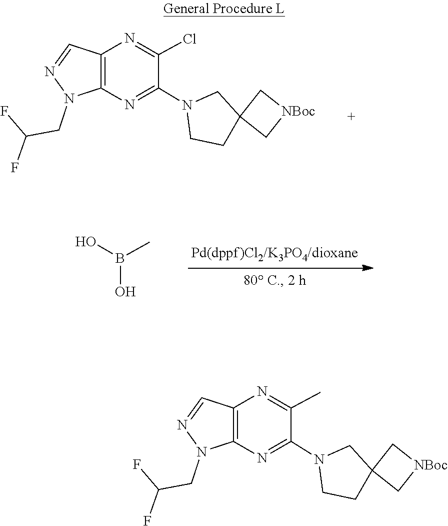

tert-butyl 6-(1-(2,2-difluoroethyl)-5-methyl-1H-pyrazolo[3,4-b]pyrazin-6-yl)-2,6-diazaspiro[3.4]octane-2-carboxylate: To a stirred solution of tert-butyl 6-[5-chloro-1-(2,2-difluoroethyl)pyrazolo[3,4-b]pyrazin-6-yl]-2,6-diazaspiro[3.4]octane-2-carboxylate (85 mg, 0.19 mmol, 1 equiv) and methylboronic acid (59.3 mg, 0.99 mmol, 5 equiv) in dioxane (1 mL) were added Na₂CO₃ (63.0 mg, 0.59 mmol, 3 equiv) and Pd(dppf)Cl₂ (14.5 mg, 0.02 mmol, 0.1 equiv) at room temperature under argon atmosphere. The resulting mixture was stirred for overnight at 80° C. under argon atmosphere. The resulting mixture was diluted with EtOAc (20 mL), washed with water (2×15 mL) and brine (15 mL), dried over anhydrous Na₂SO₄. After filtration, the filtrate was concentrated under reduced pressure. The residue was purified by Combi-flash (40 g silico gel column), eluted with Hex:EtOAc, to afford tert-butyl 6-[1-(2,2-difluoroethyl)-5-methylpyrazolo[3,4-b]pyrazin-6-yl]-2,6-diazaspiro[3.4]octane-2-carboxylate (40 mg, 51.4%) as a white solid.

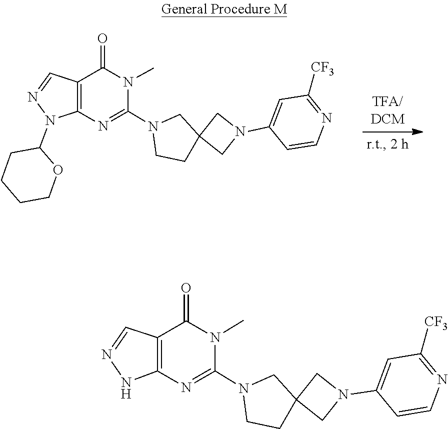

5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: A solution of 5-methyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (280 mg, 0.531 mmol, 1.00 equiv) in TFA (2 mL) and DCM (2 mL) was stirred for 2 h at room temperature under air atmosphere. After removing the solvent, the residual was basified by TEA (1.5 mL). The resulting mixture was purified by reverse flash chromatography with the following conditions: column, C18 silica gel; mobile phase, MeCN in Water, 5% to 95% gradient in 20 min; detector, UV 254/220 nm. This resulted in 5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (150 mg, 70.0%) as a white solid.

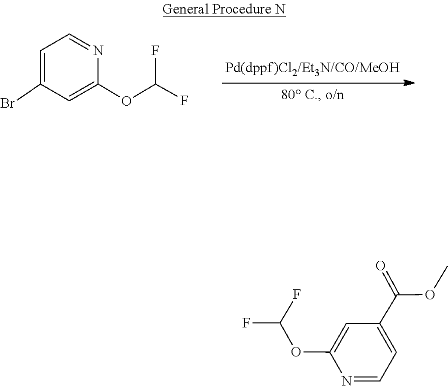

methyl 2-(difluoromethoxy)isonicotinate: A mixture of 4-bromo-2-(difluoromethoxy)pyridine (500 mg, 2.24 mmol, 1.00 equiv), Pd(dppf)Cl2 (174 mg, 0.24 mmol, 0.100 equiv) and Et₃N (678 mg, 6.72 mmol, 3.00 equiv) in MeOH (15 mL) was stirred for overnight at 80° C. under carbon dioxide atmosphere (50 atm). The resulting mixture was concentrated under reduced pressure. The residue was purified by Combi-flash (40 g silico gel column), eluted with Hex:EtOAc, to afford methyl 2-(difluoromethoxy)pyridine-4-carboxylate (320 mg, 70.3%) as a white solid.

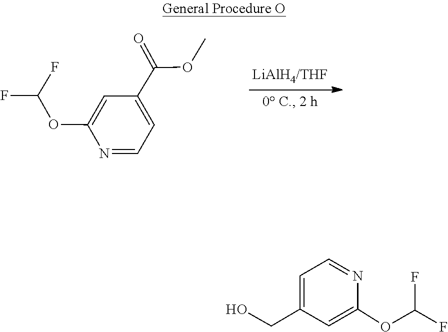

(2-(difluoromethoxy)pyridin-4-yl)methanol: To a stirred solution of methyl 2-(difluoromethoxy)pyridine-4-carboxylate (320 mg, 1.58 mmol, 1.00 equiv) in THF (4 mL) was added LiAlH₄ (1.58 mmol, 1M in THF, 1.00 equiv) dropwise at 0° C. under air atmosphere. The reaction was quenched with water (0.1 mL), 10% NaOH (0.2 mL) and water (0.3 mL) sequentially. The resulting mixture was filtrated trough celite, washed by EtOAc (3×5 mL). The combined organic layers were dried over anhydrous Na₂SO₄. After filtration, the filtrate was concentrated under reduced pressure. This resulted in (2-(difluoromethoxy)pyridin-4-yl)methanol (200 mg, 72.3%) as a colorless oil.

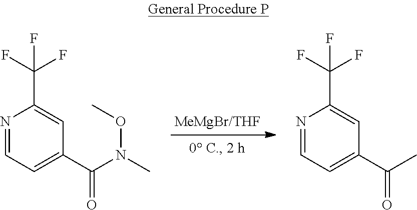 [SMILES: CON(C)C(=O)c1ccnc(C(F)(F)F)c1 ; CC(=O)c1ccnc(C(F)(F)F)c1]

1-(2-(trifluoromethyl)pyridin-4-yl)ethan-1-one: A solution of N-methoxy-N-methyl-2-(trifluoromethyl)isonicotinamide (500 mg, 2.13 mmol, 1 equiv) in THF (10 mL) was treated with methyl magnesium bromide (2.13 mL, 3M in THF, 6.41 mmol, 3 equiv) for 1 h at 0° C. under argon atmosphere. The reaction was quenched with saturated NH₄Cl aq. (10 mL) at 0° C. The resulting mixture was extracted with EtOAc (3×10 mL). The combined organic layers were washed with brine (20 mL), dried over anhydrous Na₂SO₄. After filtration, the filtrate was concentrated under reduced pressure. The residue was purified by Combi-flash (40 g silico gel column), eluted with Hex:EtOAc, to afford 1-(2-(trifluoromethyl)pyridin-4-yl)ethan-1-one (400 mg, 99.0%) as a white solid.

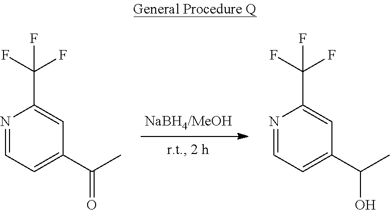

1-(2-(trifluoromethyl)pyridin-4-yl)ethan-1-ol: A solution of 1-(2-(trifluoromethyl)pyridin-4-yl)ethan-1-one (400 mg, 2.12 mmol, 1 equiv) in MeOH (4 mL) was added NaBH₄ (400 mg, 10.6 mmol, 5 equiv) in portions at 0° C. The resulting mixture was allowed to stir for 2 h at 0° C. under nitrogen atmosphere. The reaction was quenched with saturated NH₄Cl aq. (10 mL) at 0° C. The resulting mixture was extracted with EtOAc (3×10 mL). The combined organic layers were washed with brine (20 mL), dried over anhydrous Na₂SO₄. After filtration, the filtrate was concentrated under reduced pressure. The residue was purified by Combi-flash (40 g silico gel column), eluted with Hex:EtOAc, to afford 1-[2-(trifluoromethyl)pyridin-4-yl]ethanol (320 mg, 79.2%) as a colorless oil.

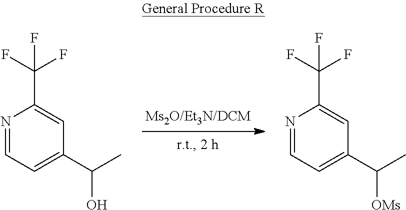 [SMILES: CC(O)c1ccnc(C(F)(F)F)c1 ; CC(OS(C)(=O)=O)c1ccnc(C(F)(F)F)c1]

1-(2-(trifluoromethyl)pyridin-4-yl)ethyl methanesulfonate: To a solution of 1-[2-(trifluoromethyl)pyridin-4-yl]ethanol (320 mg, 1.67 mmol, 1 equiv) and Et₃N (339 mg, 3.35 mmol, 2 equiv) in DCM (4 mL) was added methanesulfonic anhydride (350 mg, 2.00 mmol, 1.2 equiv) at 0° C. in portions. The resulting mixture was stirred for 2 h at 0° C. under nitrogen atmosphere. The resulting mixture was concentrated under reduced pressure. The residue was purified by Combi-flash (40 g silico gel column), eluted with Hex:EtOAc. The fractions were collected, and concentrated to give product 1-(2-(trifluoromethyl)pyridin-4-yl)ethyl methanesulfonate (200 mg, 44.3%) as a colorless oil.

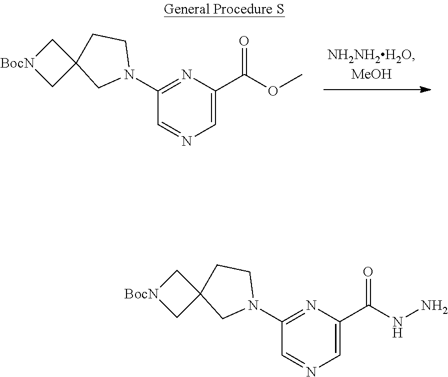 [SMILES: COC(=O)c1cncc(N2CCC3(CN(C(=O)OC(C)(C)C)C3)C2)n1 ; CC(C)(C)OC(=O)N1CC2(CCN(c3cncc(C(=O)NN)n3)C2)C1]

tert-butyl 6-(6-(hydrazinecarbonyl)pyrazin-2-yl)-2,6-diazaspiro[3.4]octane-2-carboxylate: A mixture of tert-butyl 6-(6-(methoxycarbonyl)pyrazin-2-yl)-2,6-diazaspiro[3.4]octane-2-carboxylate (500 mg, 1.44 mmol, 1.00 equiv) in MeOH (3.00 mL) was added hydrazine hydrate (86.0 mg, 1.72 mmol, 1.20 equiv) at 0° C. under N₂ atmosphere. The mixture was stirred at room temperature for 2 h. Desired product could be detected by LCMS. The residue was purified by reverse flash chromatography with the following conditions (column, C18 gel; mobile phase, B phase: MeCN, A phase: water; 5% to 100% B gradient in 20 min; detector: UV 254/220 nm). This resulted in tert-butyl 6-(6-(hydrazinecarbonyl)pyrazin-2-yl)-2,6-diazaspiro[3.4]octane-2-carboxylate (450 mg, 90.0%) as a white solid.

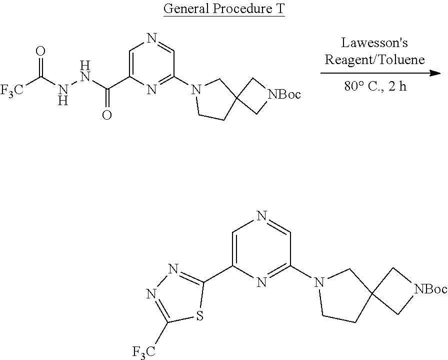 [SMILES: CC(C)(C)OC(=O)N1CC2(CCN(c3cncc(C(=O)NNC(=O)C(F)(F)F)n3)C2)C1 ; CC(C)(C)OC(=O)N1CC2(CCN(c3cncc(-c4nnc(C(F)(F)F)s4)n3)C2)C1]

tert-butyl 6-(6-(5-(trifluoromethyl)-1,3,4-thiadiazol-2-yl)pyrazin-2-yl)-2,6-diazaspiro[3.4]octane-2-carboxylate: A mixture of tert-butyl 6-(6-(2-(2,2,2-trifluoroacetyl)hydrazine-1-carbonyl)pyrazin-2-yl)-2,6-diazaspiro[3.4]octane-2-carboxylate (500 mg, 1.13 mmol, 1.00 equiv) in Toluene (5.00 mL) was added Lawesson's Reagent (273 mg, 0.674 mmol, 0.600 equiv) under N₂ atmosphere. The resulting mixture was stirred for 2 h at 80° C. under nitrogen atmosphere. Desired product could be detected by LCMS. The reaction system was concentrated and the residue was purified by Combi-flash (40 g silico gel column), eluted with DCM:MeOH. The fractions were collected, and concentrated to give product tert-butyl 6-(6-(5-(trifluoromethyl)-1,3,4-thiadiazol-2-yl)pyrazin-2-yl)-2,6-diazaspiro[3.4]octane-2-carboxylate (90 mg, 18.1%) as a yellow green solid.

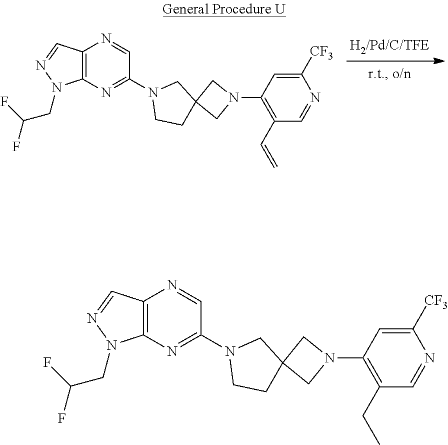 [SMILES: C=Cc1cnc(C(F)(F)F)cc1N1CC2(CCN(c3cnc4cnn(CC(F)F)c4n3)C2)C1 ; CCc1cnc(C(F)(F)F)cc1N1CC2(CCN(c3cnc4cnn(CC(F)F)c4n3)C2)C1]

1-(2,2-difluoroethyl)-6-(2-(5-ethyl-2-(trifluoromethyl)pyridin-4-yl)-2,6-diazaspiro[3.4]octan-6-yl)-1H-pyrazolo[3,4-b]pyrazine: To the solution of 1-(2,2-difluoroethyl)-6-(2-(2-(trifluoromethyl)-5-vinylpyridin-4-yl)-2,6-diazaspiro[3.4]octan-6-yl)-1H-pyrazolo[3,4-b]pyrazine (100 mg, 0.215 mmol, 1 equiv) in 2,2,2-trifluoroethanol (3 mL) was added Pd/C (10 mg, 10% Pd on carbon, wetted with water). The resulted mixture was hydrogenated overnight at room temperature. Desired product could be detected by LCMS. The reaction system was filtrated through celite and the filtrate was concentrated. The residue was purified by Combi-flash (12 g silico gel column), eluted with Hex:EtOAc. The fractions were collected, and concentrated to give product 1-(2,2-difluoroethyl)-6-(2-(5-ethyl-2-(trifluoromethyl)pyridin-4-yl)-2,6-diazaspiro[3.4]octan-6-yl)-1H-pyrazolo[3,4-b]pyrazine (40.4 mg, 40.2%) as a white solid.

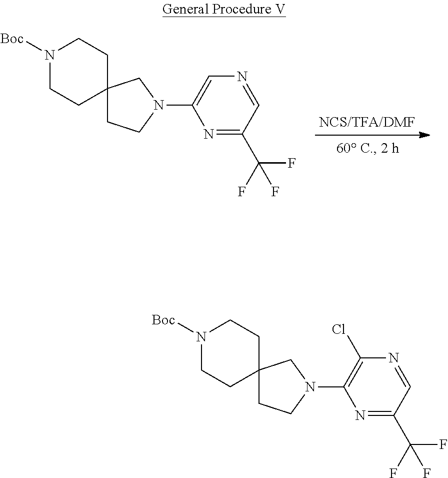 [SMILES: CC(C)(C)OC(=O)N1CCC2(CC1)CCN(c1cncc(C(F)(F)F)n1)C2 ; CC(C)(C)OC(=O)N1CCC2(CC1)CCN(c1nc(C(F)(F)F)cnc1Cl)C2]

tert-butyl 2-(3-chloro-6-(trifluoromethyl)pyrazin-2-yl)-2,8-diazaspiro[4.5]decane-8-carboxylate: To the solution of tert-butyl 2-(6-(trifluoromethyl)pyrazin-2-yl)-2,8-diazaspiro[4.5]decane-8-carboxylate (200 mg, 0.518 mmol, 1 equiv) in DMF (3 mL) was added N-chlorosuccinimide (62.2 mg, 0.466 mmol, 0.9 eq) and TFA (6 mg, 0.051 mmol, 0.1 eq). The resulted mixture was stirred for 2 h at 60° C. Desired product could be detected by LCMS. The mixture was diluted with EtOAc (15 mL), washed with water and brine, dried, evaporated, and purified with Combi-flash (40 g silico gel column), eluted with Hex:EtOAc. The fractions were collected, and concentrated to give product tert-butyl 2-(3-chloro-6-(trifluoromethyl)pyrazin-2-yl)-2,8-diazaspiro[4.5]decane-8-carboxylate (170 mg, 78.1%) as a white solid.

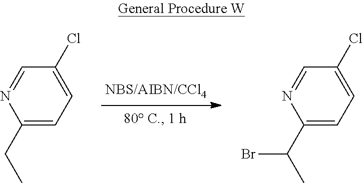

2-(1-bromoethyl)-5-chloropyridine: To the solution of 5-chloro-2-ethylpyridine (150 mg, 1.06 mmol, 1 equiv) in CCl₄ (3 mL) was added N-bromosuccinimide (283 mg, 1.59 mmol, 1.5 eq) and AIBN (174 mg, 10.6 mmol, 1 eq). The resulted mixture was stirred for 1 h at 80° C. Desired product could be detected by LCMS. The mixture was diluted with EtOAc (15 mL), washed with water and brine, dried, evaporated, and purified with Combi-flash (40 g silico gel column), eluted with Hex:EtOAc. The fractions were collected, and concentrated to give product 2-(1-bromoethyl)-5-chloropyridine (150 mg, 64.6%) as a colorless oil.

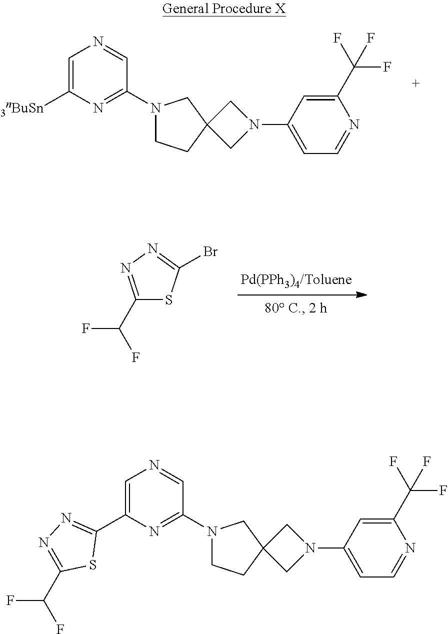

A mixture of 6-(6-(tributylstannyl)pyrazin-2-yl)-2-(2-(trifluoromethyl)pyridin-4-yl)-2,6-diazaspiro[3.4]octane (50 mg, 80 μmol, 1.0 equiv.), 2-bromo-5-(difluoromethyl)-1,3,4-thiadiazole (20.5 mg, 96 μmol, 1.2 equiv.) and Pd(PPh₃)₄ (9.2 mg, 8.0 μmol, 0.1 equiv.) in toluene (1 mL) was heated at 80° C. overnight. The reaction was monitored by LCMS. The mixture was diluted with water (5 mL), and extracted with EtOAc (5 mL×2). The organic layer was combined, washed with brine, dried, evaporated, and purified with a Combi-flash (12 g silico gel column), eluted with Hex:EtOAc. The fractions were collected, and concentrated to give tert-butyl 2-[4-(trifluoromethyl)pyridin-2-yl]-2,7-diazaspiro[3.5]nonane-7-carboxylate (5.8 mg, 15.4%) as a white solid.

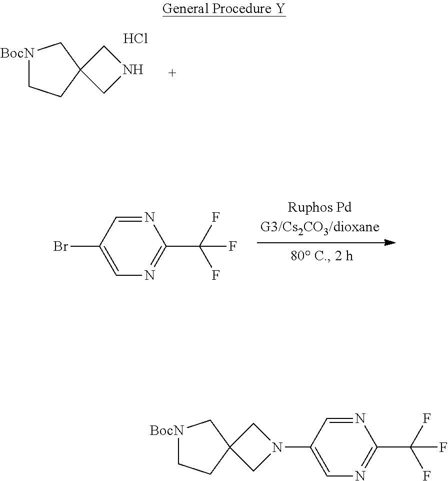 [SMILES: Cl ; CC(C)(C)OC(=O)N1CCC2(CNC2)C1 ; FC(F)(F)c1ncc(Br)cn1 ; CC(C)(C)OC(=O)N1CCC2(C1)CN(c1cnc(C(F)(F)F)nc1)C2]

tert-butyl 2-(2-(trifluoromethyl)pyrimidin-5-yl)-2,6-diazaspiro[3.4]octane-6-carboxylate: A solution of tert-butyl 2,6-diazaspiro[3.4]octane-6-carboxylate (10 g, 47.1 mmol, 1 equiv), 5-bromo-2-(trifluoromethyl)pyrimidine(16.0 g, 70.6 mmol, 1.5 equiv), RuPhos Pd G3 (1.97 g, 2.35 mmol, 0.05 equiv) and Cs₂CO₃ (46.0 g, 141 mmol, 3 equiv) in dioxane (100 mL) was stirred for 2 h at 100° C. The resulting mixture was diluted with EtOAc (200 mL). The combined organic layers were washed with water (3×200 mL) and brine (100 mL), dried over anhydrous Na₂SO₄. After filtration, the filtrate was concentrated under reduced pressure. The residue was purified with a Combi-flash (330 g silico gel column), eluted with a gradient of Hex/EtOAc. The fractions were collected, and concentrated to give tert-butyl 2-(2-(trifluoromethyl)pyrimidin-5-yl)-2,6-diazaspiro[3.4]octane-6-carboxylate (16 g, 94.8%) as a yellow solid.

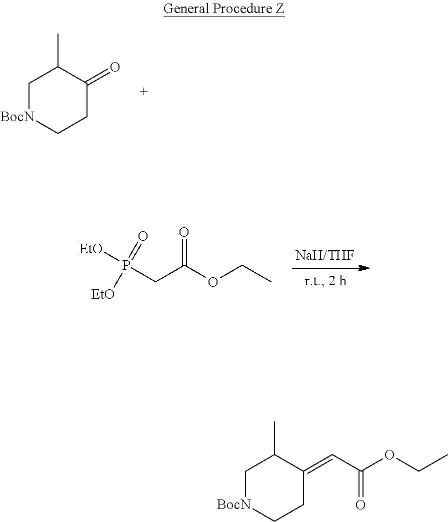 [SMILES: CC1CN(C(=O)OC(C)(C)C)CCC1=O ; CCOC(=O)CP(=O)(OCC)OCC ; CCOC(=O)/C=C1\CCN(C(=O)OC(C)(C)C)CC1C]

tert-butyl 4-(2-ethoxy-2-oxoethylidene)-3-methylpiperidine-1-carboxylate: To a stirred mixture of ethyl 2-(diethoxyphosphoryl)acetate (2.31 g, 10.3 mmol, 1.10 equiv.) in THF (20.0 mL) was added NaH (60% w/w, 356 mg, 8.89 mmol, 0.95 equiv.) in portions at 0° C. under N₂ atmosphere. The reaction system was stirred at 0° C. for 10 min followed by adding tert-butyl 3-methyl-4-oxopiperidine-1-carboxylate (2 g, 9.39 mmol, 1.00 equiv) in THF (10.0 mL) dropwise at 0° C. under N₂ atmosphere. The resulting mixture was allowed to warm to room temperature and was stirred for 2 h under N₂ atmosphere. Desired product could be detected by LCMS. The reaction was quenched with saturated NH₄Cl aq. at 0° C. The resulting mixture was extracted with EtOAc (3×15 mL). The combined organic layers were washed with water and brine dried, evaporated, and purified with a Combi-flash (80 g silico gel column), eluted with a gradient of Hex/EtOAc to give tert-butyl 4-(2-ethoxy-2-oxoethylidene)-3-methylpiperidine-1-carboxylate (1.60 g, 60.2%) as a colorless oil.

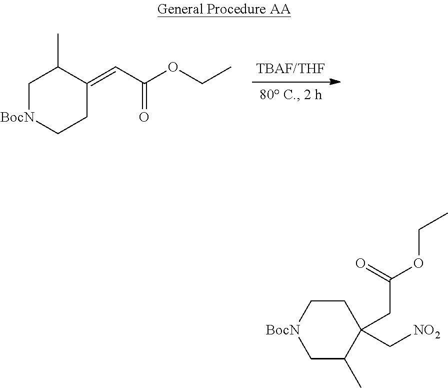 [SMILES: CCOC(=O)/C=C1\CCN(C(=O)OC(C)(C)C)CC1C ; CCOC(=O)CC1(C[N+](=O)[O-])CCN(C(=O)OC(C)(C)C)CC1C]

tert-butyl 4-(2-ethoxy-2-oxoethyl)-3-methyl-4-(nitromethyl)piperidine-1-carboxylate: To a stirred mixture of nitromethane (1.29 g, 21.2 mmol, 10 equiv.) in TBAF in THF (1 M, 10.5 mL, 5 equiv.) was added tert-butyl 4-(2-ethoxy-2-oxoethylidene)-3-methylpiperidine-1-carboxylate (600 mg, 2.12 mmol, 1.00 equiv) in THF (1 mL) dropwise at 0° C. under N₂ atmosphere. The resulting mixture was allowed to warm to 60° C. and was stirred overnight. Desired product could be detected by LCMS. The resulting mixture was concentrated and the residue was purified with a Combi-flash (40 g silico gel column), eluted with a gradient of Hex/EtOAc to give tert-butyl 4-(2-ethoxy-2-oxoethyl)-3-methyl-4-(nitromethyl)piperidine-1-carboxylate (300 mg, 41%) as a colorless oil

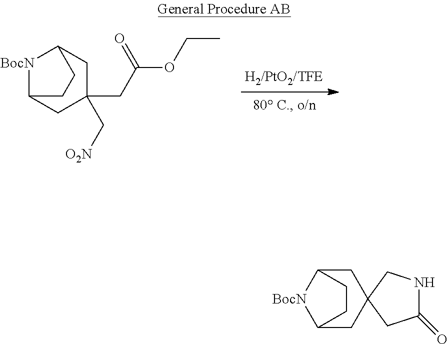 [SMILES: CCOC(=O)CC1(C[N+](=O)[O-])CC2CCC(C1)N2C(=O)OC(C)(C)C ; CC(C)(C)OC(=O)N1C2CCC1CC1(CNC(=O)C1)C2]

tert-butyl 5′-oxo-8-azaspiro[bicyclo[3.2.1]octane-3,3′-pyrrolidine]-8-carboxylate: To the solution of tert-butyl 3-(2-ethoxy-2-oxoethyl)-3-(nitromethyl)-8-azabicyclo[3.2.1]octane-8-carboxylate (300 mg, 0.842 mmol, 1 equiv.) in 2,2,2-trifluoroethanol (3 mL) was added PtO₂ (30 mg, 10 w %). The resulted mixture was hydrogenated (30 atm) overnight at 80° C. Desired product could be detected by LCMS. The reaction system was filtrated through celite and the filtrate was concentrated. The resulting mixture was purified by reverse flash chromatography with the following conditions: column, C18 silica gel; mobile phase, MeCN in Water, 5% to 95% gradient in 20 min; detector, UV 254/220 nm. This resulted in tert-butyl 5′-oxo-8-azaspiro[bicyclo[3.2.1]octane-3,3′-pyrrolidine]-8-carboxylate (150 mg, 54%) as a colorless oil.

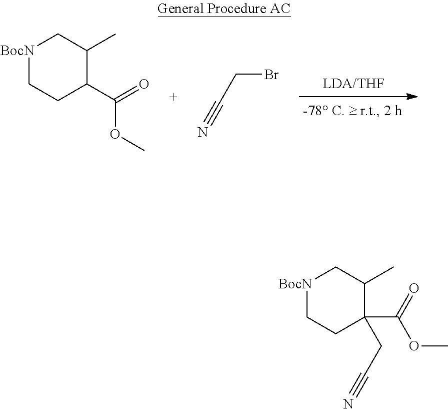

To a stirred solution of 1-(tert-butyl) 4-methyl 3-methylpiperidine-1,4-dicarboxylate (1.00 g, 3.89 mmol, 1.00 equiv.) in THF (10 mL) under argon and cooled to −78° C. was added a solution of LDA in THF (1 M, 11.7 mmol, 11.7 mL, 3 equiv.) in dropwise. The mixture was warmed up to −40° C. stirred for 0.5 h. The mixture was then cooled to −78° C. and 2-bromoacetonitrile (925 mg, 7.79 mmol, 2 equiv.) was added. The mixture was warmed to ambient temperature and stirred for 2 h. Desired product could be detected by LCMS. The reaction was quenched with saturated NH₄Cl aq. at 0° C. The resulting mixture was extracted with EtOAc (3×10 mL). The combined organic layers were washed with water and brine dried, evaporated, and purified with a Combi-flash (80 g silico gel column), eluted with a gradient of Hex/EtOAc to give 1-(tert-butyl) 4-methyl 4-(cyanomethyl)-3-methylpiperidine-1,4-dicarboxylate (250 mg, 22%) as a brown oil.

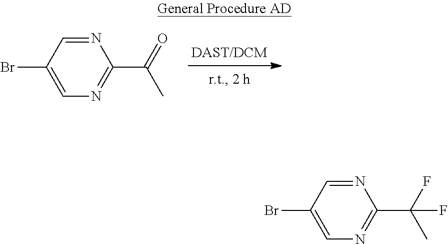

5-bromo-2-(1,1-difluoroethyl)pyrimidine: To a stirred solution of 1-(5-bromopyrimidin-2-yl)ethan-1-one (200 mg, 1.00 mmol, 1.00 equiv.) in DCM (3.00 mL) was added DAST (1.61 g, 10.0 mmol, 10.0 equiv) dropwise at 0° C. under N₂ atmosphere. The resulting mixture was stirred for 2 h at room temperature. Desired product could be detected by LCMS. The reaction was quenched with water at 0° C. The resulting mixture was extracted with EtOAc (3×20 mL). The combined organic layers were washed with water and brine, dried, evaporated, and purified with a Combi-flash (80 g silico gel column), eluted with a gradient of Hex/EtOAc to give 5-bromo-2-(1,1-difluoroethyl)pyrimidine (120 mg, 54%) as an off-white solid.

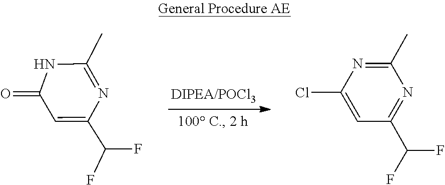

4-chloro-6-(difluoromethyl)-2-methylpyrimidine: To a stirred solution of 6-(difluoromethyl)-2-methylpyrimidin-4(3H)-one (400 mg, 2.50 mmol, 1.00 equiv.) in POCl₃ (4.00 mL) was added DIPEA (990 mg, 7.50 mmol, 3.00 equiv.) dropwise at 0° C. under N₂ atmosphere. The resulting mixture was stirred for 2 h at 100° C. Desired product could be detected by LCMS. The resulting mixture was concentrated under vacuum, neutralized with saturated NaHCO₃aq., and was extracted with DCM (3×10 mL). The combined organic layers were washed with water and brine, dried, evaporated, and purified with a Combi-flash (40 g silico gel column), eluted with a gradient of Hex/DCM to give 4-chloro-6-(difluoromethyl)-2-methylpyrimidine (300 mg, 67%) as a colorless liquid.

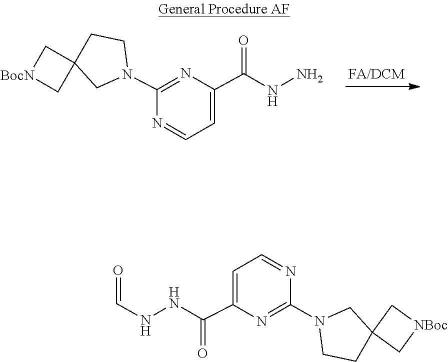 [SMILES: CC(C)(C)OC(=O)N1CC2(CCN(c3nccc(C(=O)NN)n3)C2)C1 ; CC(C)(C)OC(=O)N1CC2(CCN(c3nccc(C(=O)NNC=O)n3)C2)C1]

tert-butyl 6-(4-(2-formylhydrazine-1-carbonyl)pyrimidin-2-yl)-2,6-diazaspiro[3.4]octane-2-carboxylate: To a stirred solution of tert-butyl 6-(4-(hydrazinecarbonyl)pyrimidin-2-yl)-2,6-diazaspiro[3.4]octane-2-carboxylate (300 mg, 0.862 mmol, 1.00 equiv.) in DCM (5.00 mL) was added formic acid (119 mg, 2.57 mmol, 3.00 equiv.). The resulting mixture was stirred overnight at 60° C. Desired product could be detected by LCMS. The resulting mixture was concentrated and was purified with a Combi-flash (40 g silico gel column), eluted with a gradient of DCM/MeOH to give tert-butyl 6-(4-(2-formylhydrazine-1-carbonyl)pyrimidin-2-yl)-2,6-diazaspiro[3.4]octane-2-carboxylate (300 mg, 67%) as a colorless oil.

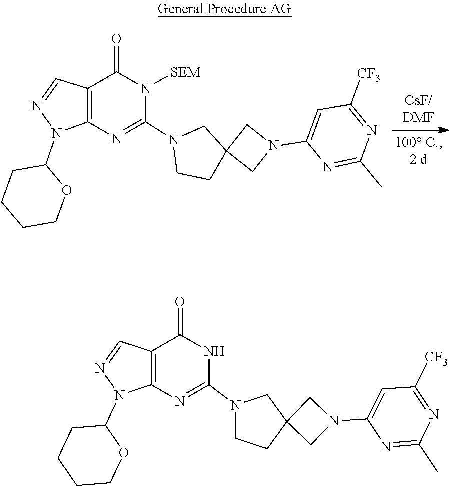

6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: A solution of 6-(2-(2-methyl-6-(trifluoromethyl)pyrimidin-4-yl)-2,6-diazaspiro[3.4]octan-6-yl)-1-(tetrahydro-2H-pyran-2-yl)-5-((2-(trimethylsilyl)ethoxy)methyl)-1,5-dihydro-4H-pyrazolo[3,4-d]pyrimidin-4-one (250 mg, 0.403 mmol, 1.00 equiv.) and CsF (760 mg, 2.01 mmol, 5.00 equiv.) in DMF (3 mL) was stirred for 2 d at 100° C. Desired product could be detected by LCMS. The resulting mixture was purified by reverse flash chromatography with the following conditions: column, C18 silica gel; mobile phase, MeCN in Water, 5% to 95% gradient in 20 min; detector, UV 254/220 nm. This resulted in 6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (170 mg, 86%) as a white solid.

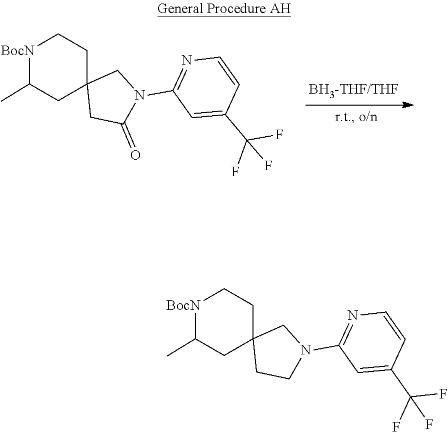 [SMILES: CC1CC2(CCN1C(=O)OC(C)(C)C)CC(=O)N(c1cc(C(F)(F)F)ccn1)C2 ; CC1CC2(CCN(c3cc(C(F)(F)F)ccn3)C2)CCN1C(=O)OC(C)(C)C]

tert-butyl 7-methyl-3-oxo-2-(4-(trifluoromethyl)pyridin-2-yl)-2,8-diazaspiro[4.5]decane-8-carboxylate: To a stirred solution of tert-butyl 7-methyl-3-oxo-2-(4-(trifluoromethyl)pyridin-2-yl)-2,8-diazaspiro[4.5]decane-8-carboxylate (120 mg, 0.290 mmol, 1.00 eq.) in THF (2 mL) under argon at 0° C. was added a solution of BH₃ in THF (1 M, 2.90 mmol, 2.9 mL, 10 equiv.) in dropwise. The mixture was warmed up to room temperature stirred overnight. Desired product could be detected by LCMS. The reaction was quenched with saturated NH₄Cl aq. at 0° C. The resulting mixture was extracted with EtOAc (3×10 mL). The combined organic layers were washed with water and brine, dried, evaporated, and purified with a Combi-flash (80 g silico gel column), eluted with a gradient of Hex/EtOAc to give tert-butyl 7-methyl-2-(4-(trifluoromethyl)pyridin-2-yl)-2,8-diazaspiro[4.5]decane-8-carboxylate (50 mg, 43%) as a white solid.

### 7-[1-(2,2-difluoroethyl)-1H-pyrazolo[3,4-b]pyrazin-6-yl]-2-[4-(trifluoromethyl)pyridin-2-yl]-2,7-diazaspiro[3.5]nonane (1)

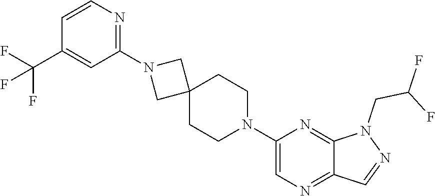 [SMILES: FC(F)Cn1ncc2ncc(N3CCC4(CC3)CN(c3cc(C(F)(F)F)ccn3)C4)nc21]

Step 1: tert-Butyl 2-[4-(trifluoromethyl)pyridin-2-yl]-2,7-diazaspiro[3.5]nonane-7-carboxylate: Followed the general procedure A using tert-butyl 2,7-diazaspiro[3.5]nonane-7-carboxylate (100 mg, 442 μmol, 1.0 equiv) and 2-bromo-4-(trifluoromethyl)pyridine (99.9 mg, 442 μmol, 1.0 equiv.) as the starting materials to give tert-butyl 2-[4-(trifluoromethyl)pyridin-2-yl]-2,7-diazaspiro[3.5]nonane-7-carboxylate (140 mg, 85%) as a colorless oil. MS m/z: 372 [M+H]⁺.

Step 2: 2-[4-(trifluoromethyl)pyridin-2-yl]-2,7-diazaspiro[3.5]nonane hydrochloride: Followed the general procedure B using tert-butyl 2-[4-(trifluoromethyl)pyridin-2-yl]-2,7-diazaspiro[3.5]nonane-7-carboxylate (102 mg) as the starting material to give the crude product 2-[4-(trifluoromethyl)pyridin-2-yl]-2,7-diazaspiro[3.5]nonane hydrochloride (102 mg). MS m/z: 272 [M+H]⁺.

Step 3: 7-[1-(2,2-difluoroethyl)-1H-pyrazolo[3,4-b]pyrazin-6-yl]-2-[4-(trifluoromethyl)pyridin-2-yl]-2,7-diazaspiro[3.5]nonane: Followed the general procedure A using 2-[4-(trifluoromethyl)pyridin-2-yl]-2,7-diazaspiro[3.5]nonane hydrochloride (145 mg, 471 μmol, 1.0 equiv) and 6-chloro-1-(2,2-difluoroethyl)-1H-pyrazolo[3,4-b]pyrazine (103 mg, 471 μmol, 1.0 equiv) as the starting materials to give 7-[l-(2,2-difluoroethyl)-1H-pyrazolo[3,4-b]pyrazin-6-yl]-2-[4-(trifluoromethyl)pyridin-2-yl]-2,7-diazaspiro[3.5]nonane (130 mg, 61%) as a white powder. ¹H NMR (500 MHz, DMSO-d₆) δ 8.50 (s, 1H), 8.29 (d, J=5.2 Hz, 1H), 8.13 (s, 1H), 6.86 (dd, J=5.3, 1.5 Hz, 1H), 6.62 (s, 1H), 6.44 (tt, J=54.9, 3.8 Hz, 1H), 4.70 (td, J=15.0, 3.8 Hz, 2H), 3.83 (s, 4H), 3.77 (dd, J=4.9, 2.8 Hz, 4H), 1.89-1.83 (m, 4H). MS m/z: 454 [M+H]⁺.

### 6-[6-(1,3,4-thiadiazol-2-yl)pyrazin-2-yl]-2-{[2-(trifluoromethyl)pyridin-3-yl]oxy}-6-azaspiro[3.5]nonane (2)

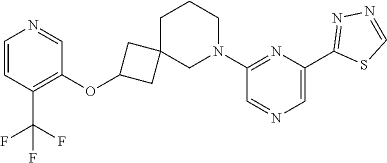 [SMILES: FC(F)(F)c1ccncc1OC1CC2(CCCN(c3cncc(-c4nncs4)n3)C2)C1]

Step 1: tert-butyl 2-{[4-(trifluoromethyl)pyridin-3-yl]oxy}-6-azaspiro[3.5]nonane-6-carboxylate: Followed the general procedure C using 4-(trifluoromethyl)pyridin-3-ol (67.6 mg, 414 μmol) and tert-butyl 2-hydroxy-6-azaspiro[3.5]nonane-6-carboxylate (100 mg, 414 μmol, 1.0 equiv.) as the starting materials to give tert-butyl 2-{[4-(trifluoromethyl)pyridin-3-yl]oxy}-6-azaspiro[3.5]nonane-6-carboxylate (100 mg, 92%) as a colorless oil MS m/z: 387 [M+H]⁺.

Step 2: 2-{[4-(trifluoromethyl)pyridin-3-yl]oxy}-6-azaspiro[3.5]nonane hydrochloride: Followed the general procedure B tert-butyl 2-{[4-(trifluoromethyl)pyridin-3-yl]oxy}-6-azaspiro[3.5]nonane-6-carboxylate (100 mg) as the starting material to give the crude product 2-{[2-(trifluoromethyl)pyridin-3-yl]oxy}-6-azaspiro[3.5]nonane hydrochloride (80 mg). MS m/z: 287 [M+H]⁺.

Step 3: 6-[6-(1,3,4-thiadiazol-2-yl)pyrazin-2-yl]-2-{[2-(trifluoromethyl)pyridin-3-yl]oxy}-6-azaspiro[3.5]nonane: Followed the general procedure A using 2-{[4-(trifluoromethyl)pyridin-3-yl]oxy}-6-azaspiro[3.5]nonanehydrochloride (24.2 mg, 85 μmol, 1.0 equiv) and 2-chloro-6-(1,3,4-thiadiazol-2-yl)pyrazine (16.8 mg, 85 μmol, 1.0 equiv.) as the starting materials to give 6-[6-(1,3,4-thiadiazol-2-yl)pyrazin-2-yl]-2-{[4-(trifluoromethyl)pyridin-3-yl]oxy}-6-azaspiro[3.5]nonane (14 mg, 37%, two isomers in a ratio of 2:3) as a colorless oil. ¹H NMR (major isomer, 500 MHz, DMSO-d₆) δ 9.71 (s, 1H), 8.63 (s, 1H), 8.60 (s, 1H), 8.49 (s, 1H), 8.40 (t, J=5.6 Hz, 2H), 7.65 (d, J=4.8 Hz, 2H), 5.26 (p, J=6.7 Hz, 1H), 3.70 (s, 2H), 3.62 (td, J=5.5, 2.0 Hz, 2H), 2.48-2.39 (m, 1H), 1.98-1.91 (m, 1H), 1.85-1.79 (m, 2H), 1.76 (dd, J=7.4, 4.4 Hz, 1H), 1.70 (dd, J=7.5, 4.4 Hz, 2H), 1.63 (dd, J=7.8, 4.0 Hz, 1H), 1.62-1.54 (m, 2H). MS m/z: 449 [M+H]⁺.

### 2-[1-(2,2-difluoroethyl)-1H-pyrazolo[3,4-b]pyrazin-6-yl]-7-[4-(trifluoromethyl)pyridin-2-yl]-2,7-diazaspiro[3.5]nonane (3)

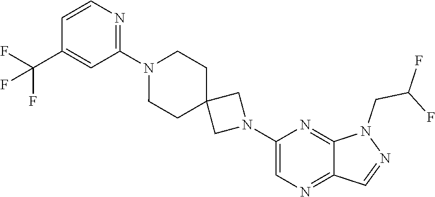 [SMILES: FC(F)Cn1ncc2ncc(N3CC4(CCN(c5cc(C(F)(F)F)ccn5)CC4)C3)nc21]

Step 1: tert-butyl 7-[4-(trifluoromethyl)pyridin-2-yl]-2,7-diazaspiro[3.5]nonane-2-carboxylate: Followed the general procedure A using tert-butyl 2,7-diazaspiro[3.5]nonane-2-carboxylate (200 mg, 884 μmol, 1.0 equiv) and 2-bromo-4-(trifluoromethyl)pyridine (200 mg, 884 μmol, 1.0 equiv.) as the starting materials to give tert-butyl 7-[4-(trifluoromethyl)pyridin-2-yl]-2,7-diazaspiro[3.5]nonane-2-carboxylate (175 mg, 53%) as a white solid. MS m/z: 372 [M+H]⁺.

Step 2: 7-[4-(trifluoromethyl)pyridin-2-yl]-2,7-diazaspiro[3.5]nonane hydrochloride: Followed the general procedure B using tert-butyl 7-[4-(trifluoromethyl)pyridin-2-yl]-2,7-diazaspiro[3.5]nonane-2-carboxylate (175 mg) as the starting material to give the crude product 7-[4-(trifluoromethyl)pyridin-2-yl]-2,7-diazaspiro[3.5]nonane hydrochloride (160 mg). MS m/z: 272 [M+H]⁺.

Step 3: 2-[1-(2,2-difluoroethyl)-1H-pyrazolo[3,4-b]pyrazin-6-yl]-7-[4-(trifluoromethyl)pyridin-2-yl]-2,7-diazaspiro[3.5]nonane: Followed the general procedure A using 7-[4-(trifluoromethyl)pyridin-2-yl]-2,7-diazaspiro[3.5]nonane hydrochloride (160 mg, 520 μmol, 1.0 equiv) and 6-chloro-1-(2,2-difluoroethyl)-1H-pyrazolo[3,4-b]pyrazine (114 mg, 520 μmol, 1.0 equiv) as the starting materials to give 2-[1-(2,2-difluoroethyl)-1H-pyrazolo[3,4-b]pyrazin-6-yl]-7-[4-(trifluoromethyl)pyridin-2-yl]-2,7-diazaspiro[3.5]nonane (113 mg, 48%) as a white powder. ¹H NMR (500 MHz, DMSO-d₆) δ 8.32 (d, J=5.2 Hz, 1H), 8.14 (s, 1H), 7.93 (s, 1H), 7.14 (s, 1H), 6.85 (dd, J=5.2, 1.4 Hz, 1H), 6.44 (tt, J=54.9, 3.8 Hz, 1H), 4.67 (td, J=15.0, 3.8 Hz, 2H), 3.98 (s, 4H), 3.71-3.60 (m, 4H), 1.83 (t, J=5.5 Hz, 4H). MS m/z: 454 [M+H]⁺.

### 6-[1-(2,2-Difluoroethyl)-1H-pyrazolo[3,4-b]pyrazin-6-yl]-2-{[2-(trifluoromethyl)pyridin-3-yl]oxy}-6-azaspiro[3.5]nonane (4)

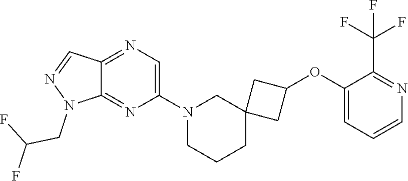 [SMILES: FC(F)Cn1ncc2ncc(N3CCCC4(CC(Oc5cccnc5C(F)(F)F)C4)C3)nc21]

Step 1: tert-butyl 2-{[2-(trifluoromethyl)308pyridine-3-yl]oxy}-6-azaspiro[3.5]nonane-6-carboxylate: Followed the general procedure C using 2-(trifluoromethyl)pyridin-3-ol (67.6 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-{[2-(trifluoromethyl)pyridin-3-yl]oxy}-6-azaspiro[3.5]nonane-6-carboxylate (148 mg, 92%) as a light-yellow oil MS m/z: 387 [M+H]⁺.

Step 2: 2-{[2-(trifluoromethyl)pyridin-3-yl]oxy}-6-azaspiro[3.5]nonane hydrochloride: Followed the general procedure B using tert-butyl 2-{[2-(trifluoromethyl)pyridin-3-yl]oxy}-6-azaspiro[3.5]nonane-6-carboxylate (148 mg) as the starting material to give the crude product 2-{[2-(trifluoromethyl)pyridin-3-yl]oxy}-6-azaspiro[3.5]nonane hydrochloride (120 mg). MS m/z: 287 [M+H]⁺.

Step 3: 6-[1-(2,2-difluoroethyl)-1H-pyrazolo[3,4-b]pyrazin-6-yl]-2-{[2-(trifluoromethyl)pyridin-3-yl]oxy}-6-azaspiro[3.5]nonane: Followed the general procedure A using 2-{[2-(trifluoromethyl)pyridin-3-yl]oxy}-6-azaspiro[3.5]nonane hydrochloride (18 mg, 55 μmol, 1.0 equiv) and 6-chloro-1-(2,2-difluoroethyl)-1H-pyrazolo[3,4-b]pyrazine (12 mg, 55 μmol, 1.0 equiv.) as the starting materials to give 6-[1-(2,2-difluoroethyl)-1H-pyrazolo[3,4-b]pyrazin-6-yl]-2-{[2-(trifluoromethyl)pyridin-3-yl]oxy}-6-azaspiro[3.5]nonane (19 mg, 74%, two isomers in a ratio of 2:3) as a colorless oil. ¹H NMR (minor isomer, 500 MHz, DMSO-d₆) δ 8.45 (s, 1H), 8.23 (dd, J=4.4, 1.2 Hz, 1H), 8.09 (s, 1H), 7.62 (dd, J=8.5, 4.5 Hz, 1H), 7.55 (dd, J=8.7, 1.2 Hz, 1H), 6.35 (tt, J=54.9, 3.8 Hz, 1H), 5.01-4.91 (m, 1H), 4.62 (td, J=14.9, 3.9 Hz, 2H), 3.78 (s, 2H), 3.70 (t, J=5.5 Hz, 2H), 2.41-2.31 (m, 2H), 2.00-1.89 (m, 2H), 1.75 (dd, J=7.5, 4.4 Hz, 2H), 1.63 (td, J=7.1, 6.6, 3.6 Hz, 2H). ¹H NMR (major isomer, 500 MHz, DMSO-d₆) δ 8.56 (s, 1H), 8.25 (dd, J=4.5, 1.2 Hz, 1H), 8.13 (s, 1H), 7.66 (dd, J=8.5, 4.5 Hz, 1H), 7.56 (d, J=8.5 Hz, 1H), 6.44 (tt, J=54.8, 3.8 Hz, 1H), 5.16 (p, J=6.7 Hz, 1H), 4.71 (td, J=15.0, 3.8 Hz, 2H), 3.79 (s, 2H), 3.70 (t, J=5.6 Hz, 2H), 2.48-2.41 (m, 2H), 1.83-1.74 (m, 2H), 1.71 (t, J=5.9 Hz, 2H), 1.58 (d, J=6.3 Hz, 2H). MS m/z: 469 [M+H]⁺.

### 7-[1-(2,2-difluoroethyl)-1H-pyrazolo[3,4-b]pyrazin-6-yl]-2-[2-(trifluoromethyl)309pyridine-3-yl]-2,7-diazaspiro[4.5]decane (5)

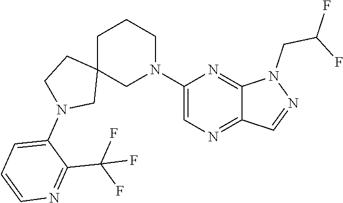 [SMILES: FC(F)Cn1ncc2ncc(N3CCCC4(CCN(c5cccnc5C(F)(F)F)C4)C3)nc21]

Step 1: tert-butyl 2-[2-(trifluoromethyl)pyridin-3-yl]-2,7-diazaspiro[4.5]decane-7-carboxylate: Followed the general procedure D using tert-butyl 2,7-diazaspiro[4.5]decane-7-carboxylate (198 mg, 825 μmol, 1.0 equiv) and 3-bromo-2-(trifluoromethyl)pyridine (280 mg, 825 μmol, 1.0 equiv.) as the starting materials to give tert-butyl 2-[2-(trifluoromethyl)pyridin-3-yl]-2,7-diazaspiro[4.5]decane-7-carboxylate (243 mg, 61%) as a yellow oil. MS m/z: 386 [M+H]⁺.

Step 2: 2-[2-(trifluoromethyl)pyridin-3-yl]-2,7-diazaspiro[4.5]decane hydrochloride: Followed the general procedure B using tert-butyl 2-[2-(trifluoromethyl)pyridin-3-yl]-2,7-diazaspiro[4.5]decane-7-carboxylate (243 mg) as the starting material to give the crude product 2-[2-(trifluoromethyl)pyridin-3-yl]-2,7-diazaspiro[4.5]decane hydrochloride (200 mg). MS m/z: 286 [M+H]⁺.

Step 3: 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decane: Followed the general procedure A using 2-[2-(trifluoromethyl)pyridin-3-yl]-2,7-diazaspiro[4.5]decane hydrochloride (17.7 mg, 55 μmol, 1.0 equiv) 6-chloro-1-(2,2-difluoroethyl)-1H-pyrazolo[3,4-b]pyrazine (12 mg, 55 μmol, 1.0 equiv) as the starting materials to give 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decane (17 mg, 66%) as a white powder. ¹H NMR (500 MHz, DMSO-d₆) δ 8.41 (s, 1H), 8.09 (s, 1H), 8.06 (dd, J=4.0, 1.4 Hz, 1H), 7.46 (qd, J=8.7, 2.7 Hz, 2H), 6.35 (tt, J=55.0, 3.9 Hz, 1H), 4.56 (tdd, J=14.8, 4.0, 1.4 Hz, 2H), 3.92-3.65 (m, 5H), 3.33-3.27 (m, 1H), 3.16 (q, J=9.7 Hz, 2H), 1.90 (ddd, J=11.7, 7.4, 3.8 Hz, 1H), 1.93-1.63 (m, 5H). MS m/z: 454 [M+H]⁺.

### 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decane (6)

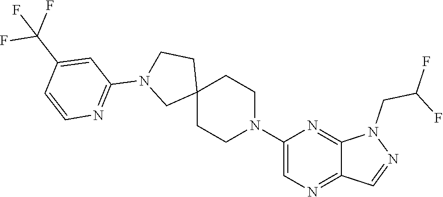 [SMILES: FC(F)Cn1ncc2ncc(N3CCC4(CCN(c5cc(C(F)(F)F)ccn5)C4)CC3)nc21]

Step 1: tert-butyl 2-[4-(trifluoromethyl)pyridin-2-yl]-2,8-diazaspiro[4.5]decane-8-carboxylate: Followed the general procedure A using tert-butyl 2,8-diazaspiro[4.5]decane-8-carboxylate (106 mg, 442 μmol, 1.0 equiv) and 2-bromo-4-(trifluoromethyl)pyridine (100 mg, 442 μmol, 1.0 equiv.) as the starting materials to give tert-butyl 2-[4-(trifluoromethyl)pyridin-2-yl]-2,8-diazaspiro[4.5]decane-8-carboxylate (115 mg, 68%) as a yellow oil. MS m/z: 386 [M+H]⁺.

Step 2: 2-[4-(trifluoromethyl)pyridin-2-yl]-2,8-diazaspiro[4.5]decane hydrochloride: Followed the general procedure B using tert-butyl 2-[4-(trifluoromethyl)pyridin-2-yl]-2,8-diazaspiro[4.5]decane-8-carboxylate (115 mg) as the starting material to give the crude product 2-[4-(trifluoromethyl)pyridin-2-yl]-2,8-diazaspiro[4.5]decane hydrochloride (88 mg). MS m/z: 286 [M+H]⁺.

Step 3: 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decane: Followed the general procedure A using 2-[4-(trifluoromethyl)pyridin-2-yl]-2,8-diazaspiro[4.5]decane hydrochloride (25.1 mg, 78 μmol, 1.0 equiv) and 6-chloro-1-(2,2-difluoroethyl)-1H-pyrazolo[3,4-b]pyrazine (17 mg, 78 μmol, 1.0 equiv) as the starting materials to give 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decane (21 mg, 58%) as a colorless oil. ¹H NMR (500 MHz, DMSO-d₆) δ 8.49 (s, 1H), 8.28 (d, J=5.2 Hz, 1H), 8.13 (s, 1H), 6.79 (dd, J=5.2, 1.5 Hz, 1H), 6.69 (d, J=1.6 Hz, 1H), 6.44 (tt, J=54.9, 3.8 Hz, 1H), 4.69 (td, J=15.0, 3.8 Hz, 2H), 3.93-3.88 (m, 2H), 3.77-3.71 (m, 2H), 3.54 (t, J=7.0 Hz, 2H), 3.42 (s, 2H), 1.94 (t, J=7.0 Hz, 2H), 1.72-1.61 (m, 4H). MS m/z: 468 [M+H]⁺.

### 2-[1-(2,2-difluoroethyl)-1H-pyrazolo[3,4-b]pyrazin-6-yl]-7-[2-(trifluoromethyl)pyridin-3-yl]-2,7-diazaspiro[4.5]decane (7)

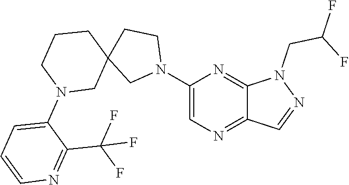 [SMILES: FC(F)Cn1ncc2ncc(N3CCC4(CCCN(c5cccnc5C(F)(F)F)C4)C3)nc21]

Step 1: tert-butyl 7-[2-(trifluoromethyl)pyridin-3-yl]-2,7-diazaspiro[4.5]decane-2-carboxylate: Followed the general procedure D using tert-butyl 2,7-diazaspiro[4.5]decane-2-carboxylate (198 mg, 825 μmol, 1.0 equiv) and 3-bromo-2-(trifluoromethyl)pyridine (280 mg, 825 μmol, 1.0 equiv.) as the starting materials to give tert-butyl 7-[2-(trifluoromethyl)pyridin-3-yl]-2,7-diazaspiro[4.5]decane-2-carboxylate (38 mg, 12%) as a white solid. MS m/z: 386 [M+H]⁺.

Step 2: 7-[2-(trifluoromethyl)pyridin-3-yl]-2,7-diazaspiro[4.5]decane hydrochloride: Followed the general procedure B using tert-butyl 7-[2-(trifluoromethyl)pyridin-3-yl]-2,7-diazaspiro[4.5]decane-2-carboxylate (38 mg) as the starting material to give the crude product 7-[2-(trifluoromethyl)pyridin-3-yl]-2,7-diazaspiro[4.5]decane hydrochloride (30 mg). MS m/z: 286 [M+H]⁺.

Step 3: 2-[1-(2,2-difluoroethyl)-1H-pyrazolo[3,4-b]pyrazin-6-yl]-7-[2-(trifluoromethyl)pyridin-3-yl]-2,7-diazaspiro[4.5]decane: Followed the general procedure A using 7-[2-(trifluoromethyl)pyridin-3-yl]-2,7-diazaspiro[4.5]decane hydrochloride (17.7 mg, 55 μmol, 1.0 equiv) 6-chloro-1-(2,2-difluoroethyl)-1H-pyrazolo[3,4-b]pyrazine (12 mg, 55 μmol, 1.0 equiv) as the starting materials to give 2-[1-(2,2-difluoroethyl)-1H-pyrazolo[3,4-b]pyrazin-6-yl]-7-[2-(trifluoromethyl)pyridin-3-yl]-2,7-diazaspiro[4.5]decane (17 mg, 66%) as a colorless oil. ¹H NMR (500 MHz, DMSO-d₆) δ 8.45 (dd, J=4.5, 1.4 Hz, 1H), 8.10 (s, 1H), 8.04 (d, J=2.4 Hz, 1H), 8.03 (s, 1H), 7.68 (dd, J=8.3, 4.5 Hz, 1H), 6.43 (tt, J=55.0, 3.9 Hz, 1H), 4.66 (tdd, J=15.0, 7.0, 3.9 Hz, 2H), 3.85 (d, J=11.2 Hz, 1H), 3.69-3.62 (m, 1H), 3.62-3.51 (m, 1H), 3.35-3.29 (m, 2H), 2.91-2.81 (m, 3H), 2.17-2.09 (m, 1H), 1.89 (q, J=11.1, 10.3 Hz, 1H), 1.78-1.56 (m, 4H). MS m/z: 468 [M+H]⁺.

### 2-[1-(2,2-difluoroethyl)-1H-pyrazolo[3,4-b]pyrazin-6-yl]-6-[2-(trifluoromethyl)pyridin-4-yl]-2,6-diazaspiro[3.5]nonane (8)

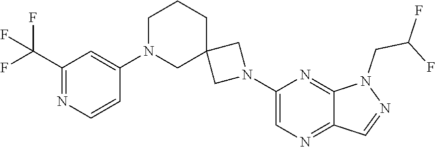 [SMILES: FC(F)Cn1ncc2ncc(N3CC4(CCCN(c5ccnc(C(F)(F)F)c5)C4)C3)nc21]

Step 1: tert-butyl 6-[2-(trifluoromethyl)pyridin-4-yl]-2,6-diazaspiro[3.5]nonane-2-carboxylate: Followed the general procedure D using tert-butyl 2,6-diazaspiro[3.5]nonane-2-carboxylate (200 mg, 884 μmol, 1.0 equiv) and 4-bromo-2-(trifluoromethyl)pyridine (200 mg, 884 μmol, 1.0 equiv.) as the starting materials to give tert-butyl 6-[2-(trifluoromethyl)pyridin-4-yl]-2,6-diazaspiro[3.5]nonane-2-carboxylate (250 mg, 76%) as a white solid. MS m/z: 372 [M+H]⁺.

Step 2: 6-[2-(trifluoromethyl)pyridin-4-yl]-2,6-diazaspiro[3.5]nonane hydrochloride: Followed the general procedure B using tert-butyl 6-[2-(trifluoromethyl)pyridin-4-yl]-2,6-diazaspiro[3.5]nonane-2-carboxylate (250 mg) as the starting material to give the crude product 6-[2-(trifluoromethyl)pyridin-4-yl]-2,6-diazaspiro[3.5]nonane hydrochloride (200 mg). MS m/z: 272 [M+H]⁺.

Step 3: 2-[1-(2,2-difluoroethyl)-1H-pyrazolo[3,4-b]pyrazin-6-yl]-6-[2-(trifluoromethyl)pyridin-4-yl]-2,6-diazaspiro[3.5]nonane: Followed the general procedure A using 6-[2-(trifluoromethyl)pyridin-4-yl]-2,6-diazaspiro[3.5]nonanehydrochloride (200 mg, 650 μmol, 1.0 equiv) 6-chloro-1-(2,2-difluoroethyl)-1H-pyrazolo[3,4-b]pyrazine (142 mg, 650 μmol, 1.0 equiv) as the starting materials to give 2-[1-(2,2-difluoroethyl)-1H-pyrazolo[3,4-b]pyrazin-6-yl]-6-[2-(trifluoromethyl)pyridin-4-yl]-2,6-diazaspiro[3.5]nonane (150 mg, 51%) as a white solid. ¹H NMR (500 MHz, DMSO-d₆) δ 8.24 (d, J=6.0 Hz, 1H), 8.14 (s, 1H), 7.94 (s, 1H), 7.33 (d, J=2.6 Hz, 1H), 7.14 (dd, J=6.1, 2.6 Hz, 1H), 6.42 (tt, J=54.9, 3.8 Hz, 1H), 4.65 (td, J=15.0, 3.8 Hz, 2H), 3.98-3.81 (m, 4H), 3.68 (s, 2H), 3.40 (t, J=5.6 Hz, 2H), 1.90 (t, J=5.9 Hz, 2H), 1.70-1.60 (m, 2H). MS m/z: 454 [M+H]⁺.

### 2-[1-(2,2-difluoroethyl)-1H-pyrazolo[3,4-b]pyrazin-6-yl]-6-[2-(trifluoromethyl)pyridin-3-yl]-2,6-diazaspiro[3.5]nonane (9)

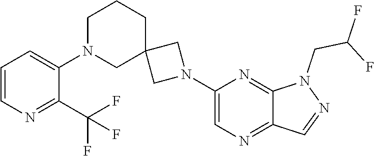 [SMILES: FC(F)Cn1ncc2ncc(N3CC4(CCCN(c5cccnc5C(F)(F)F)C4)C3)nc21]

Step 1: tert-butyl 6-[2-(trifluoromethyl)pyridin-3-yl]-2,6-diazaspiro[3.5]nonane-2-carboxylate: Followed the general procedure D using tert-butyl 2,6-diazaspiro[3.5]nonane-2-carboxylate (50 mg, 221 μmol, 1.0 equiv) and 3-bromo-2-(trifluoromethyl)pyridine (50 mg, 221 μmol, 1.0 equiv.) as the starting materials to give tert-butyl 6-[2-(trifluoromethyl)pyridin-3-yl]-2,6-diazaspiro[3.5]nonane-2-carboxylate (11 mg, 13%) as a white solid. MS m/z: 372 [+H]⁺.

Step 2: 6-[2-(trifluoromethyl)pyridin-3-yl]-2,6-diazaspiro[3.5]nonane hydrochloride: Followed the general procedure B using tert-butyl 6-[2-(trifluoromethyl)pyridin-3-yl]-2,6-diazaspiro[3.5]nonane-2-carboxylate (11 mg) as the starting material to give the crude product 6-[2-(trifluoromethyl)pyridin-3-yl]-2,6-diazaspiro[3.5]nonane hydrochloride (12 mg). MS m/z: 272 [M+H]⁺.

Step 3: 2-[1-(2,2-difluoroethyl)-1H-pyrazolo[3,4-b]pyrazin-6-yl]-6-[2-(trifluoromethyl)pyridin-4-yl]-2,6-diazaspiro[3.5]nonane: Followed the general procedure A using 6-[2-(trifluoromethyl)pyridin-3-yl]-2,6-diazaspiro[3.5]nonane hydrochloride (17 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 2-[1-(2,2-difluoroethyl)-1H-pyrazolo[3,4-b]pyrazin-6-yl]-6-[2-(trifluoromethyl)pyridin-3-yl]-2,6-diazaspiro[3.5]nonane (17 mg, 68%) as a colorless oil. ¹H NMR (500 MHz, DMSO-d₆) δ 8.48 (dd, J=4.5, 1.4 Hz, 1H), 8.12 (s, 1H), 8.08 (dd, J=8.3, 1.4 Hz, 1H), 7.93 (s, 1H), 7.72 (dd, J=8.3, 4.5 Hz, 1H), 6.42 (tt, J=54.9, 3.8 Hz, 1H), 4.66 (td, J=15.1, 3.8 Hz, 2H), 3.94 (q, J=8.7 Hz, 4H), 3.10 (s, 2H), 2.80 (t, J=5.2 Hz, 2H), 1.82 (s, 2H), 1.69 (q, J=5.5 Hz, 2H). MS m/z: 454 [M+H]⁺.

### 6-[1-(2,2,2-trifluoroethyl)-1H-pyrazolo[3,4-b]pyrazin-6-yl]-2-{[2-(trifluoromethyl)pyridin-3-yl]oxy}-6-azaspiro[3.5]nonane (10)

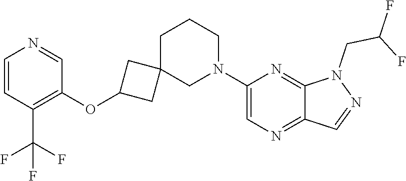 [SMILES: FC(F)Cn1ncc2ncc(N3CCCC4(CC(Oc5cnccc5C(F)(F)F)C4)C3)nc21]

Followed the general procedure A using 2-{[4-(trifluoromethyl)pyridin-3-yl]oxy}-6-azaspiro[3.5]nonane hydrochloride (22 mg, 69 μmol, 1.0 equiv) and 6-chloro-1-(2,2-difluoroethyl)-1H-pyrazolo[3,4-b]pyrazine (15 mg, 69 μmol, 1.0 equiv) as the starting materials to give 6-[1-(2,2-difluoroethyl)-1H-pyrazolo[3,4-b]pyrazin-6-yl]-2-{[4-(trifluoromethyl)pyridin-3-yl]oxy}-6-azaspiro[3.5]nonane (28 mg, 87%, two isomers in a ratio of 2:3) as a colorless oil. ¹H NMR (major isomer, 500 MHz, DMSO-d₆) δ 8.57 (s, 1H), 8.48 (s, 1H), 8.44 (d, J=3.9 Hz, 1H), 8.40 (J=4.3 Hz, 1H), 8.13 (s, 1H), 8.09 (s, 1H), 7.65 (d, J=4.8 Hz, 2H), 6.44 (tt, J=54.9, 3.8 Hz, 2H), 5.29 (p, J=6.7 Hz, 1H), 4.71 (td, J=15.1, 3.8 Hz, 2H), 3.79 (s, 2H), 3.70 (t, J=5.5 Hz, 2H), 2.49-2.44 (m, 1H), 2.44-2.33 (m, 1H), 1.99-1.92 (m, 1H), 1.87-1.49 (m, 5H). MS m/z: 469 [M+H]⁺.

### 6-[1-(2,2,2-trifluoroethyl)-1H-pyrazolo[3,4-b]pyrazin-6-yl]-2-{[2-(trifluoromethyl)pyridin-3-yl]oxy}-6-azaspiro[3.5]nonane (11)

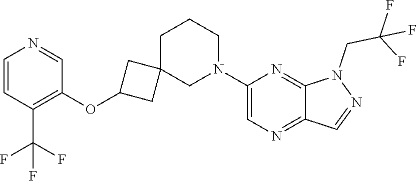 [SMILES: FC(F)(F)Cn1ncc2ncc(N3CCCC4(CC(Oc5cnccc5C(F)(F)F)C4)C3)nc21]

Followed the general procedure A using 2-{[4-(trifluoromethyl)pyridin-3-yl]oxy}-6-azaspiro[3.5]nonane hydrochloride (24 mg, 74.4 μmol, 1.0 equiv) and 6-chloro-1-(2,2,2-trifluoroethyl)-1H-pyrazolo[3,4-b]pyrazine (17.6 mg, 74.4 μmol, 1.0 equiv) as the starting materials to give 6-[1-(2,2,2-trifluoroethyl)-1H-pyrazolo[3,4-b]pyrazin-6-yl]-2-{[4-(trifluoromethyl)pyridin-3-yl]oxy}-6-azaspiro[3.5]nonane (14 mg, 39%, two isomers in a ratio of 2:3) as a colorless oil. ¹H NMR (major isomer, 500 MHz, DMSO-d₆) δ 8.59 (s, 1H), 8.47 (s, 1H), 8.43 (s, 1H), 8.40 (d, J=5.3 Hz, 1H), 8.18 (s, 1H), 7.65 (dd, J=4.9, 1H), 5.34-5.01 (m, 3H), 3.82 (s, 2H), 3.77-3.67 (m, 2H), 2.47-2.35 (m, 2H), 2.04-1.93 (m, 2H), 1.86-1.53 (m, 6H). MS m/z: 487 [M+H]⁺.

### 6-[1-(2,2-difluoroethyl)-1H-pyrazolo[3,4-b]pyrazin-6-yl]-2-[2-(trifluoromethyl)pyridin-4-yl]-2,6-diazaspiro[3.5]nonane (12)

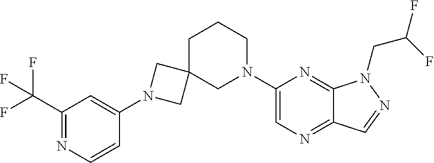 [SMILES: FC(F)Cn1ncc2ncc(N3CCCC4(CN(c5ccnc(C(F)(F)F)c5)C4)C3)nc21]

Step 1: tert-butyl 2-[2-(trifluoromethyl)pyridin-4-yl]-2,6-diazaspiro[3.5]nonane-6-carboxylate: Followed the general procedure D using tert-butyl 2,6-diazaspiro[3.5]nonane-6-carboxylate (50 mg, 221 μmol, 1.0 equiv) and 4-bromo-2-(trifluoromethyl)pyridine (50 mg, 221 μmol, 1.0 equiv.) as the starting materials to give tert-butyl 2-[2-(trifluoromethyl)pyridin-4-yl]-2,6-diazaspiro[3.5]nonane-6-carboxylate (20 mg, 24%) as a white solid. MS m/z: 372 [M+H]⁺.

Step 2: 7-[4-(trifluoromethyl)pyridin-2-yl]-2,7-diazaspiro[3.5]nonane hydrochloride: Followed the general procedure B using tert-butyl 2-[2-(trifluoromethyl)pyridin-4-yl]-2,6-diazaspiro[3.5]nonane-6-carboxylate (20 mg) as the starting material to give the crude product 2-[2-(trifluoromethyl)pyridin-4-yl]-2,6-diazaspiro[3.5]nonane hydrochloride (20 mg). MS m/z: 272 [M+H]⁺.

Step 3: 6-[1-(2,2-difluoroethyl)-1H-pyrazolo[3,4-b]pyrazin-6-yl]-2-[2-(trifluoromethyl)pyridin-4-yl]-2,6-diazaspiro[3.5]nonane: Followed the general procedure A using 2-[2-(trifluoromethyl)pyridin-4-yl]-2,6-diazaspiro[3.5]nonane hydrochloride (20 mg, 65 μmol, 1.0 equiv) and 6-chloro-1-(2,2-difluoroethyl)-1H-pyrazolo[3,4-b]pyrazine (14.2 mg, 65 μmol, 1.0 equiv) as the starting materials to give 6-[1-(2,2-difluoroethyl)-1H-pyrazolo[3,4-b]pyrazin-6-yl]-2-[2-(trifluoromethyl)pyridin-4-yl]-2,6-diazaspiro[3.5]nonane (18 mg, 61%) as a colorless oil. ¹H NMR (500 MHz, DMSO-d₆) δ 8.56 (s, 1H), 8.21 (d, J=5.6 Hz, 1H), 8.13 (s, 1H), 6.73 (d, J=2.2 Hz, 1H), 6.54 (dd, J=5.7, 2.3 Hz, 1H), 6.40 (tt, J=54.9, 3.8 Hz, 1H), 4.67 (td, J=15.1, 3.8 Hz, 2H), 3.98 (s, 2H), 3.80 (d, J=8.2 Hz, 2H), 3.74 (t, J=5.6 Hz, 2H), 3.70 (d, J=8.3 Hz, 2H), 1.96-1.87 (m, 2H), 1.65 (dt, J=7.2, 3.0 Hz, 2H). MS m/z: 454 [M+H]⁺.

### 6-[1-(2,2-difluoroethyl)-1H-pyrazolo[3,4-b]pyrazin-6-yl]-2-{[2-(trifluoromethyl)pyridin-4-yl]oxy}-6-azaspiro[3.5]nonane (13)

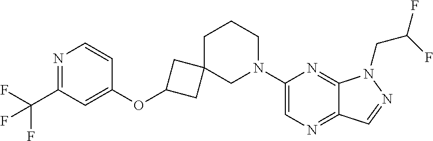 [SMILES: FC(F)Cn1ncc2ncc(N3CCCC4(CC(Oc5ccnc(C(F)(F)F)c5)C4)C3)nc21]

Step 1: tert-butyl 2-{[2-(trifluoromethyl)pyridin-4-yl]oxy}-6-azaspiro[3.5]nonane-6-carboxylate: Followed the general procedure C using 2-(trifluoromethyl)pyridin-4-ol (67.6 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-{[2-(trifluoromethyl)pyridin-4-yl]oxy}-6-azaspiro[3.5]nonane-6-carboxylate (105 mg, 66%) as a light-yellow oil. MS m/z: 387 [M+H]⁺.

Step 2: 2-{[2-(trifluoromethyl)pyridin-4-yl]oxy}-6-azaspiro[3.5]nonane hydrochloride: Followed the general procedure B using tert-butyl 2-{[2-(trifluoromethyl)pyridin-4-yl]oxy}-6-azaspiro[3.5]nonane-6-carboxylate (105 mg) as the starting material to give the crude product 2-{[2-(trifluoromethyl)pyridin-4-yl]oxy}-6-azaspiro[3.5]nonane hydrochloride (80 mg). MS m/z: 287 [M+H]⁺.

Step 3: 6-[1-(2,2-difluoroethyl)-1H-pyrazolo[3,4-b]pyrazin-6-yl]-2-{[2-(trifluoromethyl)pyridin-4-yl]oxy}-6-azaspiro[3.5]nonane: Followed the general procedure A using 2-{[2-(trifluoromethyl)pyridin-4-yl]oxy}-6-azaspiro[3.5]nonane hydrochloride (22.1 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-{[2-(trifluoromethyl)pyridin-4-yl]oxy}-6-azaspiro[3.5]nonane (28 mg, 87%, two isomers in a ratio of 2:3) as a colorless oil. ¹H NMR (minor isomer, 500 MHz, DMSO-d₆) δ 8.45 (s, 1H), 8.23 (dd, J=4.4, 1.2 Hz, 1H), 8.09 (s, 1H), 7.62 (dd, J=8.5, 4.5 Hz, 1H), 7.55 (dd, J=8.7, 1.2 Hz, 1H), 6.35 (tt, J=54.9, 3.8 Hz, 1H), 5.01-4.91 (m, 1H), 4.62 (td, J=14.9, 3.9 Hz, 2H), 3.78 (s, 2H), 3.70 (t, J=5.5 Hz, 2H), 2.41-2.31 (m, 2H), 2.00-1.89 (m, 2H), 1.75 (dd, J=7.5, 4.4 Hz, 2H), 1.63 (td, J=7.1, 6.6, 3.6 Hz, 2H). ¹H NMR (major isomer, 500 MHz, DMSO-d₆) δ 8.57 (s, 1H), 8.55 (d, J=5.7 Hz, 1H), 8.13 (s, 1H), 7.32 (t, J=2.4 Hz, 1H), 7.16 (dd, J=5.8, 2.4 Hz, 1H), 6.44 (tt, J=54.9, 3.8 Hz, 1H), 5.20 (p, J=6.9 Hz, 1H), 4.71 (td, J=15.1, 3.8 Hz, 3H), 3.79 (s, 2H), 3.70 (td, J=5.5, 2.2 Hz, 2H), 2.49-2.43 (m, 2H), 2.03-1.94 (m, 1H), 1.90-1.79 (m, 1H), 1.79-1.73 (m, 1H), 1.70 (dd, J=7.5, 4.4 Hz, 1H), 1.67-1.62 (m, 1H), 1.57 (dt, J=7.1, 3.5 Hz, 1H). MS m/z: 469 [M+H]⁺.

### 2-[1-(2,2-difluoroethyl)-1H-pyrazolo[3,4-b]pyrazin-6-yl]-6-[4-(trifluoromethyl)pyridin-2-yl]-2,6-diazaspiro[3.5]nonane (14)

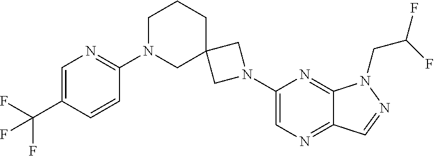 [SMILES: FC(F)Cn1ncc2ncc(N3CC4(CCCN(c5ccc(C(F)(F)F)cn5)C4)C3)nc21]

Step 1: tert-butyl 2-[1-(2,2-difluoroethyl)-1H-pyrazolo[3,4-b]pyrazin-6-yl]-2,6-diazaspiro[3.5]nonane-6-carboxylate: Followed the general procedure A using tert-butyl 2,6-diazaspiro[3.5]nonane-6-carboxylate hydrochloride (120 mg, 457 μmol, 1.0 equiv) and 6-chloro-1-(2,2-difluoroethyl)-1H-pyrazolo[3,4-b]pyrazine (100 mg, 457 μmol, 1.0 equiv.) as the starting materials to give tert-butyl 2-[1-(2,2-difluoroethyl)-1H-pyrazolo[3,4-b]pyrazin-6-yl]-2,6-diazaspiro[3.5]nonane-6-carboxylate (180 mg, 96%) as a colorless oil. MS m/z: 409 [M+H]⁺.

Step 2: 2-[1-(2,2-difluoroethyl)-1H-pyrazolo[3,4-b]pyrazin-6-yl]-2,6-diazaspiro[3.5]nonane hydrochloride: Followed the general procedure B using tert-butyl 2-[1-(2,2-difluoroethyl)-1H-pyrazolo[3,4-b]pyrazin-6-yl]-2,6-diazaspiro[3.5]nonane-6-carboxylate (180 mg) as the starting material to give the crude product 2-[4-(trifluoromethyl)pyridin-2-yl]-2,7-diazaspiro[3.5]nonane hydrochloride (170 mg). MS m/z: 309 [M+H]⁺.

Step 3: 2-[1-(2,2-difluoroethyl)-1H-pyrazolo[3,4-b]pyrazin-6-yl]-6-[5-(trifluoromethyl)pyridin-2-yl]-2,6-diazaspiro[3.5]nonane: Followed the general procedure A using 2-[1-(2,2-difluoroethyl)-1H-pyrazolo[3,4-b]pyrazin-6-yl]-2,6-diazaspiro[3.5]nonane hydrochloride (20 mg, 58 μmol, 1.0 equiv) and 2-bromo-5-(trifluoromethyl)pyridine (13.1 mg, 58 μmol, 1.0 equiv) as the starting materials to give 2-[1-(2,2-difluoroethyl)-1H-pyrazolo[3,4-b]pyrazin-6-yl]-6-[5-(trifluoromethyl)pyridin-2-yl]-2,6-diazaspiro[3.5]nonane (130 mg, 61%) as a colorless oil. ¹H NMR (500 MHz, DMSO-d₆) δ 8.37 (dd, J=1.8, 0.9 Hz, 1H), 8.14 (s, 1H), 7.93 (s, 1H), 7.76 (dd, J=9.2, 2.6 Hz, 1H), 7.07 (d, J=9.2 Hz, 1H), 6.42 (tt, J=54.9, 3.8 Hz, 1H), 4.65 (td, J=15.0, 3.8 Hz, 2H), 3.97-3.79 (m, 4H), 3.74-3.43 (m, 4H), 1.91 (t, J=6.0 Hz, 2H), 1.67-1.54 (m, 2H). MS m/z: 454 [M+H]⁺.

### 6-[1-(2,2-difluoroethyl)-1H-pyrazolo[3,4-b]pyrazin-6-yl]-2-[5-(trifluoromethyl)pyridin-3-yl]-2,6-diazaspiro[3.5]nonane (15)

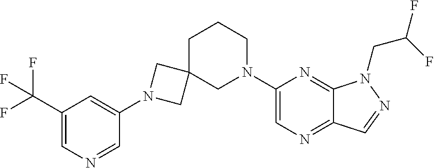 [SMILES: FC(F)Cn1ncc2ncc(N3CCCC4(CN(c5cncc(C(F)(F)F)c5)C4)C3)nc21]

Followed the general procedure D using 6-[1-(2,2-difluoroethyl)-1H-pyrazolo[3,4-b]pyrazin-6-yl]-2,6-diazaspiro[3.5]nonane hydrochloride (20 mg, 58 μmol, 1.0 equiv) and 3-bromo-5-(trifluoromethyl)pyridine (13.1 mg, 58 μmol, 1.0 equiv.) as the starting materials to give 6-[1-(2,2-difluoroethyl)-1H-pyrazolo[3,4-b]pyrazin-6-yl]-2-[5-(trifluoromethyl)pyridin-3-yl]-2,6-diazaspiro[3.5]nonane (13 mg, 49%) as a colorless oil. ¹H NMR (500 MHz, DMSO-d₆) δ 8.55 (s, 1H), 8.21-8.17 (m, 1H), 8.13 (s, 1H), 8.06 (d, J=2.7 Hz, 1H), 7.06 (t, J=2.4 Hz, 1H), 6.40 (tt, J=54.9, 3.8 Hz, 2H), 4.67 (td, J=15.1, 3.8 Hz, 2H), 3.99 (s, 2H), 3.79 (d, J=7.7 Hz, 2H), 3.74 (t, J=5.6 Hz, 2H), 3.67 (d, J=7.7 Hz, 2H), 1.95-1.88 (m, 2H), 1.68-1.61 (m, 2H). MS m/z: 454 [M+H]⁺.

### 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decane (16)

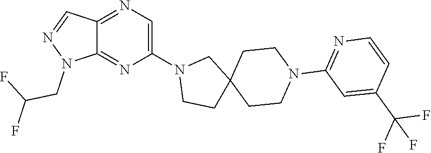 [SMILES: FC(F)Cn1ncc2ncc(N3CCC4(CCN(c5cc(C(F)(F)F)ccn5)CC4)C3)nc21]

Step 1: tert-butyl 8-[4-(trifluoromethyl)pyridin-2-yl]-2,8-diazaspiro[4.5]decane-2-carboxylate: Followed the general procedure A using tert-butyl 2,8-diazaspiro[4.5]decane-2-carboxylate (106 mg, 442 μmol, 1.0 equiv) and 2-bromo-4-(trifluoromethyl)pyridine (100 mg, 442 μmol, 1.0 equiv.) as the starting materials to give tert-butyl 8-[4-(trifluoromethyl)pyridin-2-yl]-2,8-diazaspiro[4.5]decane-2-carboxylate (130 mg, 76%) as a colorless oil. MS m/z: 386 [M+H]⁺.

Step 2: 8-[4-(trifluoromethyl)pyridin-2-yl]-2,8-diazaspiro[4.5]decane hydrochloride: Followed the general procedure B using tert-butyl 8-[4-(trifluoromethyl)pyridin-2-yl]-2,8-diazaspiro[4.5]decane-2-carboxylate (130 mg) as the starting material to give the crude product 2-[4-(trifluoromethyl)pyridin-2-yl]-2,7-diazaspiro[3.5]nonane hydrochloride (100 mg). MS m/z: 286 [M+H]⁺.

Step 3: 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decane: Followed the general procedure A using 8-[4-(trifluoromethyl)pyridin-2-yl]-2,8-diazaspiro[4.5]decane hydrochloride (25 mg, 78 μmol, 1.0 equiv) and 6-chloro-1-(2,2-difluoroethyl)-1H-pyrazolo[3,4-b]pyrazine (17 mg, 78 μmol, 1.0 equiv) as the starting materials to give 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decane (18 mg, 49%) as a white solid. ¹H NMR (500 MHz, DMSO-d₆) δ 8.32 (d, J=5.1 Hz, 1H), 7.11 (d, J=1.5 Hz, 1H), 6.84 (dd, J=5.2, 1.4 Hz, 1H), 6.45 (tt, J=55.0, 3.9 Hz, 1H), 4.68 (td, J=14.9, 3.9 Hz, 2H), 3.72 (br s, 2H), 3.68 (t, J=7.0 Hz, 4H), 3.54 (s, 2H), 1.96 (br s, 2H), 1.71-1.55 (m, 4H). MS m/z: 468 [M+H]⁺.

### 6-[1-(2,2-difluoroethyl)-1H-pyrazolo[3,4-b]pyrazin-6-yl]-2-[4-(trifluoromethyl)pyridin-3-yl]-2,6-diazaspiro[3.5]nonane (17)

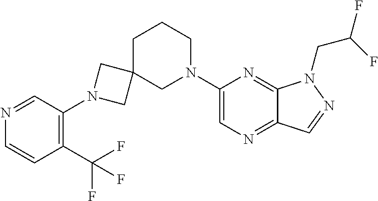 [SMILES: FC(F)Cn1ncc2ncc(N3CCCC4(C3)CN(c3cnccc3C(F)(F)F)C4)nc21]

Followed the general procedure D using 6-[1-(2,2-difluoroethyl)-1H-pyrazolo[3,4-b]pyrazin-6-yl]-2,6-diazaspiro[3.5]nonane hydrochloride (20 mg, 58 μmol, 1.0 equiv) and 3-bromo-4-(trifluoromethyl)pyridine (13.1 mg, 58 μmol, 1.0 equiv.) as the starting materials to give 6-[1-(2,2-difluoroethyl)-1H-pyrazolo[3,4-b]pyrazin-6-yl]-2-[4-(trifluoromethyl)pyridin-3-yl]-2,6-diazaspiro[3.5]nonane (14 mg, 53%) as a colorless oil. ¹H NMR (500 MHz, DMSO-d₆) δ 8.55 (s, 1H), 8.13 (s, 1H), 8.01 (s, 1H), 7.99 (d, J=5.1 Hz, 1H), 7.40 (d, J=5.2 Hz, 1H), 6.41 (tt, J=54.9, 3.8 Hz, 1H), 4.67 (td, J=15.0, 3.8 Hz, 2H), 3.99 (s, 2H), 3.92 (d, J=7.8 Hz, 2H), 3.81 (d, J=7.8 Hz, 2H), 3.73 (t, J=5.5 Hz, 2H), 1.95-1.90 (m, 2H), 1.67-1.64 (m, 2H). MS m/z: 454 [M+H]⁺.

### 6-[1-(2,2-difluoroethyl)-1H-pyrazolo[3,4-b]pyrazin-6-yl]-2-[3-(trifluoromethyl)pyridine-2-yl]-2,6-diazaspiro[3.5]nonane (18)

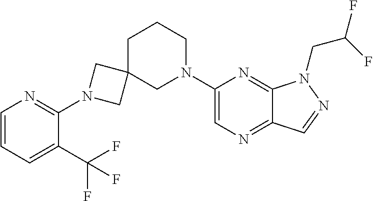 [SMILES: FC(F)Cn1ncc2ncc(N3CCCC4(C3)CN(c3ncccc3C(F)(F)F)C4)nc21]

Followed the general procedure A using 6-[1-(2,2-difluoroethyl)-1H-pyrazolo[3,4-b]pyrazin-6-yl]-2,6-diazaspiro[3.5]nonane hydrochloride (20 mg, 58 μmol, 1.0 equiv) and 2-bromo-3-(trifluoromethyl)pyridine (13 mg, 58 μmol, 1.0 equiv) as the starting materials to give 6-[1-(2,2-difluoroethyl)-1H-pyrazolo[3,4-b]pyrazin-6-yl]-2-[3-(trifluoromethyl)pyridin-2-yl]-2,6-diazaspiro[3.5]nonane (15 mg, 57%) as a white solid. ¹H NMR (500 MHz, DMSO-d₆) δ 8.55 (s, 1H), 8.33 (dd, J=4.8, 1.7 Hz, 1H), 8.13 (s, 1H), 7.86 (dd, J=7.7, 1.7 Hz, 1H), 6.77 (dd, J=7.7, 4.8 Hz, 1H), 6.40 (tt, J=54.9, 3.8 Hz, 1H), 4.67 (td, J=15.0, 3.8 Hz, 2H), 3.97 (s, 2H), 3.91 (d, J=8.6 Hz, 2H), 3.80 (d, J=8.6 Hz, 2H), 3.73 (t, J=5.6 Hz, 2H), 1.90 (t, J=5.9 Hz, 2H), 1.66 (tt, J=8.0, 4.3 Hz, 2H). MS m/z: 454 [M+H]⁺.

### 6-[1-(2,2-difluoroethyl)-1H-pyrazolo[3,4-b]pyrazin-6-yl]-2-phenyl-2,6-diazaspiro[3.5]nonane (19)

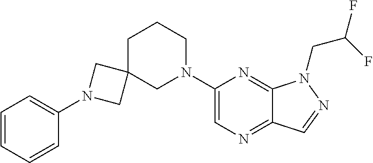 [SMILES: FC(F)Cn1ncc2ncc(N3CCCC4(CN(c5ccccc5)C4)C3)nc21]

Step 1: tert-butyl 2-phenyl-2,6-diazaspiro[3.5]nonane-6-carboxylate: Followed the general procedure F using tert-butyl 2,6-diazaspiro[3.5]nonane-6-carboxylate hydrochloride (500 mg, 1.9 mmol, 1 equiv.) and iodobenzene (776 mg, 1.9 mmom, 1 equiv.) as the starting materials to give tert-butyl 2-phenyl-2,6-diazaspiro[3.5]nonane-6-carboxylate (200 mg, 35%) a white solid. MS m/z: 303 [M+H]⁺.

Step 2: 2-phenyl-2,6-diazaspiro[3.5]nonane hydrochloride: Followed the general procedure B using tert-butyl 2-phenyl-2,6-diazaspiro[3.5]nonane-6-carboxylate (200 mg) as the starting material to give the crude product 2-[4-(trifluoromethyl)pyridin-2-yl]-2,7-diazaspiro[3.5]nonane hydrochloride (180 mg). MS m/z: 203 [M+H]⁺.

Step 3: 6-[1-(2,2-difluoroethyl)-1H-pyrazolo[3,4-b]pyrazin-6-yl]-2-phenyl-2,6-diazaspiro[3.5]nonane: Followed the general procedure A using 2-phenyl-2,6-diazaspiro[3.5]nonane hydrochloride (13.1 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 6-[1-(2,2-difluoroethyl)-1H-pyrazolo[3,4-b]pyrazin-6-yl]-2-phenyl-2,6-diazaspiro[3.5]nonane (15 mg, 71%) as a colorless oil. ¹H NMR (500 MHz, DMSO-d₆) δ 8.54 (s, 1H), 8.12 (s, 1H), 7.14 (dd, J=8.5, 7.3 Hz, 2H), 6.65 (ddd, J=7.3, 6.2, 1.1 Hz, 1H), 6.44-6.39 (m, 2H), 6.40 (tt, J=54.9, 3.8 Hz, 1H), 4.67 (td, J=15.1, 3.8 Hz, 2H), 3.97 (s, 2H), 3.74 (t, J=5.6 Hz, 2H), 3.62 (d, J=7.1 Hz, 2H), 3.48 (d, J=7.1 Hz, 2H), 1.89 (dd, J=7.4, 4.6 Hz, 2H), 1.73-1.56 (m, 2H). MS m/z: 385 [M+H]⁺.

### 2-[1-(2,2-difluoroethyl)-1H-pyrazolo[3,4-b]pyrazin-6-yl]-6-[6-(trifluoromethyl)pyridin-3-yl]-2,6-diazaspiro[3.5]nonane (20)

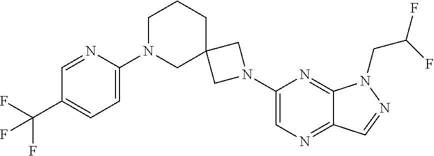 [SMILES: FC(F)Cn1ncc2ncc(N3CC4(CCCN(c5ccc(C(F)(F)F)cn5)C4)C3)nc21]

Followed the general procedure D using 2-[1-(2,2-difluoroethyl)-1H-pyrazolo[3,4-b]pyrazin-6-yl]-2,6-diazaspiro[3.5]nonane hydrochloride (20 mg, 58 μmol, 1.0 equiv) and 5-bromo-2-(trifluoromethyl)pyridine (13.1 mg, 58 μmol, 1.0 equiv.) as the starting materials to give 2-[1-(2,2-difluoroethyl)-1H-pyrazolo[3,4-b]pyrazin-6-yl]-6-[6-(trifluoromethyl)pyridin-3-yl]-2,6-diazaspiro[3.5]nonane (8 mg, 30%) as a colorless oil. ¹H NMR (500 MHz, DMSO-d₆) δ 8.50 (d, J=2.9 Hz, 1H), 8.14 (s, 1H), 7.94 (s, 1H), 7.62 (d, J=8.8 Hz, 1H), 7.52 (dd, J=8.9, 2.9 Hz, 1H), 6.43 (tt, J=54.9, 3.8 Hz, 1H), 4.66 (td, J=15.0, 3.8 Hz, 2H), 4.01-3.84 (m, 4H), 3.57 (s, 2H), 3.32-3.24 (m, 2H), 1.87 (t, J=5.9 Hz, 2H), 1.69 (t, J=6.0 Hz, 2H). MS m/z: 454 [M+H]⁺.

### 2-[1-(2,2-difluoroethyl)-1H-pyrazolo[3,4-b]pyrazin-6-yl]-6-[4-(trifluoromethyl)pyridin-2-yl]-2,6-diazaspiro[3.5]nonane (21)

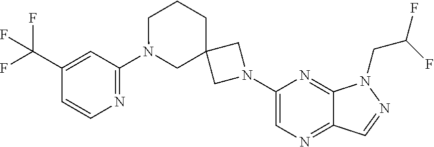 [SMILES: FC(F)Cn1ncc2ncc(N3CC4(CCCN(c5cc(C(F)(F)F)ccn5)C4)C3)nc21]

Followed the general procedure A using 2-[1-(2,2-difluoroethyl)-1H-pyrazolo[3,4-b]pyrazin-6-yl]-2,6-diazaspiro[3.5]nonane hydrochloride (75 mg, 218 μmol, 1.0 equiv) and 2-bromo-4-(trifluoromethyl)pyridine (49 mg, 218 μmol, 1.0 equiv) as the starting materials to give 2-[1-(2,2-difluoroethyl)-1H-pyrazolo[3,4-b]pyrazin-6-yl]-6-[4-(trifluoromethyl)pyridin-2-yl]-2,6-diazaspiro[3.5]nonane (8 mg, 8%) as a colorless oil. ¹H NMR (500 MHz, DMSO-d₆) δ 8.29 (d, J=5.1 Hz, 1H), 8.14 (s, 1H), 7.93 (s, 1H), 7.20 (s, 1H), 6.85 (dd, J=5.1, 1.3 Hz, 1H), 6.42 (tt, J=54.9, 3.8 Hz, 2H), 4.65 (td, J=15.0, 3.8 Hz, 2H), 3.96-3.81 (m, 6H), 3.57 (t, J=5.6 Hz, 2H), 1.90 (dd, J=7.7, 4.2 Hz, 2H), 1.62 (s, 2H). MS m/z: 454 [M+H]⁺.

### 6-[1-(2,2-difluoroethyl)-1H-pyrazolo[3,4-b]pyrazin-6-yl]-2-{[6-(trifluoromethyl)pyridin-3-yl]oxy}-6-azaspiro[3.5]nonane (22)

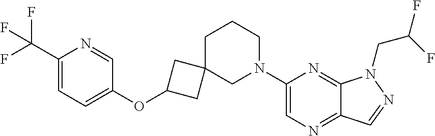 [SMILES: FC(F)Cn1ncc2ncc(N3CCCC4(CC(Oc5ccc(C(F)(F)F)nc5)C4)C3)nc21]

Step 1: tert-butyl 2-{[6-(trifluoromethyl)pyridin-3-yl]oxy}-6-azaspiro[3.5]nonane-6-carboxylate: Followed the general procedure C using 6-(trifluoromethyl)pyridin-3-ol (67.6 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-{[6-(trifluoromethyl)pyridin-3-yl]oxy}-6-azaspiro[3.5]nonane-6-carboxylate (88 mg, 55%) as a light-yellow oil. MS m/z: 387 [M+H]⁺.

Step 2: 2-{[6-(trifluoromethyl)pyridin-3-yl]oxy}-6-azaspiro[3.5]nonane hydrochloride: Followed the general procedure B tert-butyl 2-{[6-(trifluoromethyl)pyridin-3-yl]oxy}-6-azaspiro[3.5]nonane-6-carboxylate (88 mg) as the starting material to give the crude product 2-{[6-(trifluoromethyl)pyridin-3-yl]oxy}-6-azaspiro[3.5]nonane hydrochloride (80 mg). MS m/z: 287 [M+H]+

Step 3: 6-[1-(2,2-difluoroethyl)-1H-pyrazolo[3,4-b]pyrazin-6-yl]-2-{[6-(trifluoromethyl)pyridin-3-yl]oxy}-6-azaspiro[3.5]nonane: Followed the general procedure A using 2-{[6-(trifluoromethyl)pyridin-3-yl]oxy}-6-azaspiro[3.5]nonane hydrochloride (22.1 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-{[6-(trifluoromethyl)pyridin-3-yl]oxy}-6-azaspiro[3.5]nonane (24 mg, 75%, two isomers in a ratio of 2:3) as a colorless oil. ¹H NMR (major isomer, 500 MHz, DMSO-d₆) δ 8.57 (s, 1H), 8.37 (d, J=2.6 Hz, 1H), 8.13 (s, 1H), 7.82 (d, J=8.7 Hz, 1H), 7.46 (dd, J=8.8, 2.9 Hz, 1H), 6.44 (tt, J=54.9, 3.8 Hz, 2H), 5.15 (p, J=6.8 Hz, 1H), 4.71 (td, J=15.1, 3.8 Hz, 2H), 3.80 (s, 2H), 3.74-3.67 (m, 2H), 2.49-2.42 (m, 2H), 1.86-1.79 (m, 2H), 1.71 (dd, J=7.4, 4.5 Hz, 2H), 1.60-1.56 (m, 2H). MS m/z: 469 [M+H]⁺.

### 6-[1-(2,2-difluoroethyl)-1H-pyrazolo[3,4-b]pyrazin-6-yl]-2-[6-(trifluoromethyl)pyridin-2-yl]-2,6-diazaspiro[3.5]nonane (23)

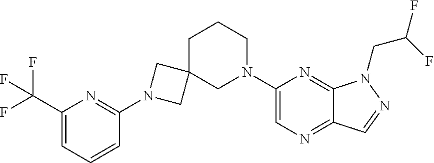 [SMILES: FC(F)Cn1ncc2ncc(N3CCCC4(CN(c5cccc(C(F)(F)F)n5)C4)C3)nc21]

Step 1: tert-butyl 2-[6-(trifluoromethyl)pyridin-2-yl]-2,6-diazaspiro[3.5]nonane-6-carboxylate: Followed the general procedure A using tert-butyl 2,8-diazaspiro[4.5]decane-2-carboxylate (50 mg, 190 μmol, 1.0 equiv) 2-bromo-6-(trifluoromethyl)pyridine (43 mg, 190 μmol, 1.0 equiv.) as the starting materials to give tert-butyl 2-[6-(trifluoromethyl)pyridin-2-yl]-2,6-diazaspiro[3.5]nonane-6-carboxylate (23 mg, 32%) as a colorless oil. MS m/z: 372 [M+H]⁺.

Step 2: 2-[6-(trifluoromethyl)pyridin-2-yl]-2,6-diazaspiro[3.5]nonane hydrochloride: Followed the general procedure B using tert-butyl 2-[6-(trifluoromethyl)pyridin-2-yl]-2,6-diazaspiro[3.5]nonane-6-carboxylate (23 mg) as the starting material to give the crude product 2-[6-(trifluoromethyl)pyridin-2-yl]-2,6-diazaspiro[3.5]nonane hydrochloride (18 mg). MS m/z: 272 [M+H]⁺.

Step 3: 6-[1-(2,2-difluoroethyl)-1H-pyrazolo[3,4-b]pyrazin-6-yl]-2-[6-(trifluoromethyl)pyridin-2-yl]-2,6-diazaspiro[3.5]nonane: Followed the general procedure A using 2-[6-(trifluoromethyl)pyridin-2-yl]-2,6-diazaspiro[3.5]nonane hydrochloride (17 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 6-[1-(2,2-difluoroethyl)-1H-pyrazolo[3,4-b]pyrazin-6-yl]-2-[6-(trifluoromethyl)pyridin-2-yl]-2,6-diazaspiro[3.5]nonane (24 mg, 96%) as a white solid. ¹H NMR (500 MHz, DMSO-d₆) δ 8.32 (d, J=5.1 Hz, 1H), 7.11 (d, J=1.5 Hz, 1H), 6.84 (dd, J=5.2, 1.4 Hz, 1H), 6.45 (tt, J=55.0, 3.9 Hz, 1H), 4.68 (td, J=14.9, 3.9 Hz, 2H), 3.72 (br s, 2H), 3.68 (t, J=7.0 Hz, 4H), 3.54 (s, 2H), 1.96 (br s, 2H), 1.71-1.55 (m, 4H). ¹H NMR (500 MHz, DMSO-d₆) δ 8.14 (s, 1H), 7.93 (s, 1H), 7.72 (dd, J=8.8, 7.2 Hz, 1H), 7.24 (d, J=8.8 Hz, 1H), 7.01 (d, J=7.3 Hz, 1H), 6.42 (tt, J=54.9, 3.8 Hz, 1H), 4.65 (td, J=15.0, 3.8 Hz, 2H), 3.92 (d, J=8.7 Hz, 2H), 3.87 (d, J=8.7 Hz, 2H), 3.81 (s, 2H), 3.56 (t, J=5.5 Hz, 2H), 1.93-1.84 (m, 2H), 1.66-1.54 (m, 2H). MS m/z: 454 [M+H]⁺.

### 6-[1-(2,2-difluoroethyl)-1H-pyrazolo[3,4-b]pyrazin-6-yl]-2-(2,5-difluorophenoxy)-6-azaspiro[3.5]nonane (24)

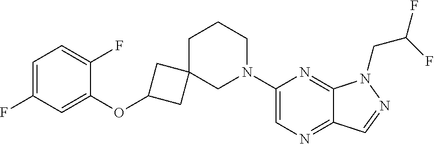 [SMILES: Fc1ccc(F)c(OC2CC3(CCCN(c4cnc5cnn(CC(F)F)c5n4)C3)C2)c1]

Step 1: tert-butyl 2-(2,5-difluorophenoxy)-6-azaspiro[3.5]nonane-6-carboxylate: Followed the general procedure C using 2,5-difluorophenol (53.9 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-(2,5-difluorophenoxy)-6-azaspiro[3.5]nonane-6-carboxylate (83 mg, 57%) as a colorless solid. MS m/z: 354 [M+H]⁺.

Step 2: 2-(2,5-difluorophenoxy)-6-azaspiro[3.5]nonane hydrochloride: Followed the general procedure B tert-butyl 2-(2,5-difluorophenoxy)-6-azaspiro[3.5]nonane-6-carboxylate (83 mg) as the starting material to give the crude product 2-(2,5-difluorophenoxy)-6-azaspiro[3.5]nonane hydrochloride (65 mg). MS m/z: 254 [M+H]⁺.

Step 3: 6-[1-(2,2-difluoroethyl)-1H-pyrazolo[3,4-b]pyrazin-6-yl]-2-{[6-(trifluoromethyl)pyridin-3-yl]oxy}-6-azaspiro[3.5]nonane: Followed the general procedure A using 2-(2,5-difluorophenoxy)-6-azaspiro[3.5]nonane hydrochloride (19.9 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-(2,5-difluorophenoxy)-6-azaspiro[3.5]nonane (28 mg, 94%, two isomers in a ratio of 3:4) as a colorless oil. ¹H NMR (major isomer, 500 MHz, DMSO-d₆) δ 8.24 (d, J=6.0 Hz, 1H), 8.14 (s, 1H), 8.03 (s, 1H), 7.94 (s, 1H), 7.33 (d, J=2.6 Hz, 1H), 7.00 (dd, J=6.1, 2.6 Hz, 1H), 6.42 (tt, J=54.9, 3.8 Hz, 3H), 4.65 (td, J=15.1, 3.8 Hz, 3H), 4.40 (p, J=6.0 Hz, 1H), 3.97-3.83 (m, 4H), 3.68 (s, 2H), 3.55-3.37 (m, 2H), 1.93-1.87 (m, 2H), 1.68-1.64 (m, 2H). MS m/z: 436 [M+H]⁺.

### 2-[1-(2,2-difluoroethyl)-1H-pyrazolo[3,4-b]pyrazin-6-yl]-6-[6-(trifluoromethyl)pyridin-2-yl]-2,6-diazaspiro[3.5]nonane (25)

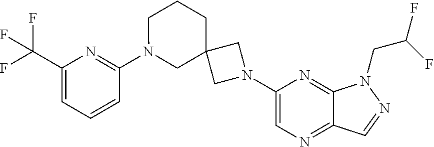 [SMILES: FC(F)Cn1ncc2ncc(N3CC4(CCCN(c5cccc(C(F)(F)F)n5)C4)C3)nc21]

Step 1: tert-butyl 2-[6-(trifluoromethyl)pyridin-2-yl]-2,6-diazaspiro[3.5]nonane-6-carboxylate: Followed the general procedure A using tert-butyl 2,8-diazaspiro[4.5]decane-2-carboxylate (50 mg, 190 μmol, 1.0 equiv) and 2-bromo-6-(trifluoromethyl)pyridine (43 mg, 190 μmol, 1.0 equiv.) as the starting materials to give tert-butyl 6-[6-(trifluoromethyl)pyridin-2-yl]-2,6-diazaspiro[3.5]nonane-2-carboxylate (57 mg, 80%) as a colorless oil. MS m/z: 372 [M+H]⁺.

Step 2: 6-[6-(trifluoromethyl)pyridin-2-yl]-2,6-diazaspiro[3.5]nonane hydrochloride: Followed the general procedure B using tert-butyl 6-[6-(trifluoromethyl)pyridin-2-yl]-2,6-diazaspiro[3.5]nonane-2-carboxylate (57 mg) as the starting material to give the crude product 6-[6-(trifluoromethyl)pyridin-2-yl]-2,6-diazaspiro[3.5]nonane hydrochloride (45 mg). MS m/z: 272 [M+H]⁺.

Step 3: 2-[1-(2,2-difluoroethyl)-1H-pyrazolo[3,4-b]pyrazin-6-yl]-6-[6-(trifluoromethyl)pyridin-2-yl]-2,6-diazaspiro[3.5]nonane: Followed the general procedure A using 6-[6-(trifluoromethyl)pyridin-2-yl]-2,6-diazaspiro[3.5]nonane hydrochloride (17 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 2-[1-(2,2-difluoroethyl)-1H-pyrazolo[3,4-b]pyrazin-6-yl]-6-[6-(trifluoromethyl)pyridin-2-yl]-2,6-diazaspiro[3.5]nonane (23 mg, 92%) as a white solid. ¹H NMR (500 MHz, DMSO-d₆) δ 8.56 (s, 1H), 8.12 (s, 1H), 7.69 (dd, J=8.5, 7.2 Hz, 1H), 7.01 (d, J=7.3 Hz, 1H), 6.63 (d, J=8.5 Hz, 1H), 6.38 (tt, J=54.9, 3.8 Hz, 1H), 4.67 (td, J=15.1, 3.8 Hz, 2H), 3.98 (s, 2H), 3.79 (d, J=8.1 Hz, 2H), 3.74 (dd, J=8.5, 3.5 Hz, 2H), 3.69 (d, J=8.2 Hz, 2H), 1.91 (t, J=5.9 Hz, 2H), 1.72-1.60 (m, 2H). MS m/z: 454 [M+H]⁺.

### 6-[1-(2,2-difluoroethyl)-1H-pyrazolo[3,4-b]pyrazin-6-yl]-2-[2-(trifluoromethyl)pyridin-3-yl]-2,6-diazaspiro[3.5]nonane (26)

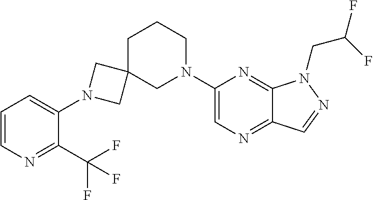 [SMILES: FC(F)Cn1ncc2ncc(N3CCCC4(C3)CN(c3cccnc3C(F)(F)F)C4)nc21]

Step 1: tert-butyl 2-[2-(trifluoromethyl)pyridin-3-yl]-2,6-diazaspiro[3.5]nonane-6-carboxylate: Followed the general procedure D using tert-butyl 2,6-diazaspiro[3.5]nonane-6-carboxylate (50 mg, 221 μmol, 1.0 equiv) and 3-bromo-2-(trifluoromethyl)pyridine (50 mg, 221 μmol, 1.0 equiv.) as the starting materials to give tert-butyl 2-[2-(trifluoromethyl)pyridin-3-yl]-2,6-diazaspiro[3.5]nonane-6-carboxylate (53 mg, 75%) as a white solid. MS m/z: 372 [M+H]⁺.

Step 2: 2-[2-(trifluoromethyl)pyridin-3-yl]-2,6-diazaspiro[3.5]nonane hydrochloride: Followed the general procedure B using tert-butyl 2-[2-(trifluoromethyl)pyridin-3-yl]-2,6-diazaspiro[3.5]nonane-6-carboxylate (53 mg) as the starting material to give the crude product 2-[2-(trifluoromethyl)pyridin-3-yl]-2,6-diazaspiro[3.5]nonane hydrochloride (45 mg). MS m/z: 272 [M+H]⁺.

Step 3: 6-[1-(2,2-difluoroethyl)-1H-pyrazolo[3,4-b]pyrazin-6-yl]-2-[2-(trifluoromethyl)pyridin-3-yl]-2,6-diazaspiro[3.5]nonane: Followed the general procedure A using 2-[2-(trifluoromethyl)pyridin-3-yl]-2,6-diazaspiro[3.5]nonane hydrochloride (17 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 6-[1-(2,2-difluoroethyl)-1H-pyrazolo[3,4-b]pyrazin-6-yl]-2-[2-(trifluoromethyl)pyridin-3-yl]-2,6-diazaspiro[3.5]nonane (6.5 mg, 26%) as a colorless oil. ¹H NMR (500 MHz, DMSO-d₆) δ 8.55 (s, 1H), 8.13 (s, 1H), 7.98 (dd, J=4.3, 1.2 Hz, 1H), 7.45 (dd, J=8.5, 4.3 Hz, 1H), 7.08 (dd, J=8.6, 1.3 Hz, 1H), 6.41 (tt, J=54.8, 3.8 Hz, 2H), 4.67 (td, J=15.0, 3.8 Hz, 2H), 3.98 (s, 2H), 3.84 (d, J=7.8 Hz, 2H), 3.73 (t, J=6.0 Hz, 4H), 1.91 (dd, J=8.3, 3.8 Hz, 2H), 1.66 (d, J=6.0 Hz, 2H). MS m/z: 454 [M+H]⁺.

### 6-[1-(2,2-difluoroethyl)-1H-pyrazolo[3,4-b]pyrazin-6-yl]-2-{[6-(trifluoromethyl)pyridin-2-yl]oxy}-6-azaspiro[3.5]nonane (27)

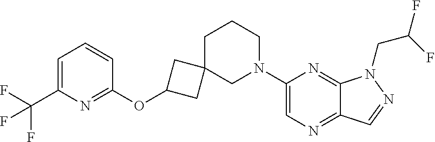 [SMILES: FC(F)Cn1ncc2ncc(N3CCCC4(CC(Oc5cccc(C(F)(F)F)n5)C4)C3)nc21]

Step 1: tert-butyl 2-{[6-(trifluoromethyl)pyridin-2-yl]oxy}-6-azaspiro[3.5]nonane-6-carboxylate: Followed the general procedure E using 2-bromo-6-(trifluoromethyl)pyridine (93.6 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-{[6-(trifluoromethyl)pyridin-2-yl]oxy}-6-azaspiro[3.5]nonane-6-carboxylate (138 mg, 86%) as a colorless oil. MS m/z: 387 [M+H]⁺.

Step 2: 2-{[6-(trifluoromethyl)pyridin-2-yl]oxy}-6-azaspiro[3.5]nonane hydrochloride: Followed the general procedure B tert-butyl 2-{[6-(trifluoromethyl)pyridin-2-yl]oxy}-6-azaspiro[3.5]nonane-6-carboxylate (138 mg) as the starting material to give the crude product 2-{[6-(trifluoromethyl)pyridin-2-yl]oxy}-6-azaspiro[3.5]nonane hydrochloride (100 mg). MS m/z: 286 [M+H]⁺.

Step 3: 6-[1-(2,2-difluoroethyl)-1H-pyrazolo[3,4-b]pyrazin-6-yl]-2-{[6-(trifluoromethyl)pyridin-2-yl]oxy}-6-azaspiro[3.5]nonane: Followed the general procedure A using 2-{[6-(trifluoromethyl)pyridin-2-yl]oxy}-6-azaspiro[3.5]nonane hydrochloride (22.1 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-{[6-(trifluoromethyl)pyridin-2-yl]oxy}-6-azaspiro[3.5]nonane (23 mg, 72%, two isomers in a ratio of 3:4) as a colorless oil. ¹H NMR (major isomer, 500 MHz, DMSO-d₆) δ 8.50 (s, 1H), 8.09 (s, 1H), 7.98-7.93 (m, 1H), 7.45 (d, J=7.4 Hz, 1H), 7.12 (ddd, J=9.5, 8.5, 0.9 Hz, 1H), 6.40 (tt, J=54.9, 3.8 Hz, 1H), 5.14 (p, J=6.9 Hz, 1H), 4.66 (td, J=14.8, 3.8 Hz, 2H), 3.77 (s, 2H), 3.70 (q, J=6.0, 5.0 Hz, 2H), 2.34-2.26 (m, 2H), 2.04-1.94 (m, 2H), 1.76 (t, J=5.9 Hz, 2H), 1.64 (td, J=6.6, 3.7 Hz, 2H). MS m/z: 469 [M+H]⁺.

### 6-[1-(2,2-difluoroethyl)-1H-pyrazolo[3,4-b]pyrazin-6-yl]-2-{[5-(trifluoromethyl)pyridin-2-yl]oxy}-6-azaspiro[3.5]nonane (28)

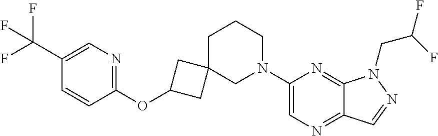 [SMILES: FC(F)Cn1ncc2ncc(N3CCCC4(CC(Oc5ccc(C(F)(F)F)cn5)C4)C3)nc21]

Step 1: tert-butyl 2-{[5-(trifluoromethyl)pyridin-2-yl]oxy}-6-azaspiro[3.5]nonane-6-carboxylate: Followed the general procedure E using 2-bromo-5-(trifluoromethyl)pyridine (93.6 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-{[5-(trifluoromethyl)pyridin-2-yl]oxy}-6-azaspiro[3.5]nonane-6-carboxylate (130 mg, 81%) as a colorless oil. MS m/z: 387 [M+H]⁺.

Step 2: 2-{[5-(trifluoromethyl)pyridin-2-yl]oxy}-6-azaspiro[3.5]nonane hydrochloride: Followed the general procedure B tert-butyl 2-{[6-(trifluoromethyl)pyridin-2-yl]oxy}-6-azaspiro[3.5]nonane-6-carboxylate (136 mg) as the starting material to give the crude product 2-{[6-(trifluoromethyl)pyridin-2-yl]oxy}-6-azaspiro[3.5]nonane hydrochloride (105 mg). MS m/z: 286 [M+H]⁺.

Step 3: 6-[1-(2,2-difluoroethyl)-1H-pyrazolo[3,4-b]pyrazin-6-yl]-2-{[5-(trifluoromethyl)pyridin-2-yl]oxy}-6-azaspiro[3.5]nonane: Followed the general procedure A using 2-{[5-(trifluoromethyl)pyridin-2-yl]oxy}-6-azaspiro[3.5]nonane hydrochloride (22.1 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-{[5-(trifluoromethyl)pyridin-2-yl]oxy}-6-azaspiro[3.5]nonane (23 mg, 72%, two isomers in a ratio of 3:4) as a colorless oil. ¹HNMR (major isomer, 500 MHz, DMSO-d₆) δ 8.58 (s, 1H), 8.39 (d, J=5.3, 1H), 8.12 (s, 1H), 7.30 (d, J=5.0, 1H), 7.17 (s, 1H), 6.45 (tt, J=54.9, 3.8 Hz, 3H), 5.41 (p, J=7.0 Hz, 1H), 4.71 (td, J=14.8, 3.8 Hz, 2H), 3.81 (s, 2H), 3.71 (d, J=5.8 Hz, 2H), 2.45-2.37 (m, 2H), 2.03-1.93 (m, 2H), 1.85-1.78 (m, 2H), 1.77-1.70 (m, 2H), 1.66-1.64 (m, 2H). MS m/z: 469 [M+H]⁺.

### 6-[1-(2,2-difluoroethyl)-1H-pyrazolo[3,4-b]pyrazin-6-yl]-2-{[4-(trifluoromethyl)pyridin-2-yl]oxy}-6-azaspiro[3.5]nonane (29)

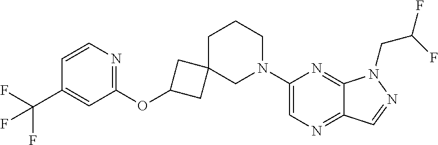 [SMILES: FC(F)Cn1ncc2ncc(N3CCCC4(CC(Oc5cc(C(F)(F)F)ccn5)C4)C3)nc21]

Step 1: tert-butyl 2-{[4-(trifluoromethyl)pyridin-2-yl]oxy}-6-azaspiro[3.5]nonane-6-carboxylate: Followed the general procedure E using 2-bromo-4-(trifluoromethyl)pyridine (93.6 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-{[5-(trifluoromethyl)pyridin-2-yl]oxy}-6-azaspiro[3.5]nonane-6-carboxylate (130 mg, 81%) as a colorless oil. MS m/z: 387 [M+H]⁺.

Step 2: 2-{[4-(trifluoromethyl)pyridin-2-yl]oxy}-6-azaspiro[3.5]nonane hydrochloride: Followed the general procedure B using tert-butyl 2-{[4-(trifluoromethyl)pyridin-2-yl]oxy}-6-azaspiro[3.5]nonane-6-carboxylate (130 mg) as the starting material to give the crude product 2-{[4-(trifluoromethyl)pyridin-2-yl]oxy}-6-azaspiro[3.5]nonane hydrochloride (100 mg). MS m/z: 286 [M+H]⁺.

Step 3: 6-[1-(2,2-difluoroethyl)-1H-pyrazolo[3,4-b]pyrazin-6-yl]-2-{[4-(trifluoromethyl)pyridin-2-yl]oxy}-6-azaspiro[3.5]nonane: Followed the general procedure A using 2-{[4-(trifluoromethyl)pyridin-2-yl]oxy}-6-azaspiro[3.5]nonane hydrochloride (22.1 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-{[4-(trifluoromethyl)pyridin-2-yl]oxy}-6-azaspiro[3.5]nonane (18 mg, 56%, two isomers in a ratio of 3:4) as a colorless oil. ¹H NMR (major isomer, 500 MHz, DMSO-d₆) δ 8.58 (s, 1H), 8.53 (s, 1H), 8.12 (s, 1H), 8.06 (dt, J=8.8, 2.7 Hz, 1H), 7.00 (t, J=8.3 Hz, 1H), 6.45 (tt, J=54.9, 3.8 Hz, 1H), 5.42 (p, J=7.1 Hz, 2H), 4.72 (td, J=14.8, 3.8 Hz, 2H), 3.81 (s, 2H), 3.70 (q, J=5.7 Hz, 2H), 2.44-2.38 (m, 2H), 2.01-1.97 (m, 2H), 1.78-1.74 (m, 2H), 1.66-1.62 (m, 2H). MS m/z: 469 [M+H]⁺.

### 2-(2,5-difluorophenoxy)-6-[1-(2,2,2-trifluoroethyl)-1H-pyrazolo[3,4-b]pyrazin-6-yl]-6-azaspiro[3.5]nonane (30)

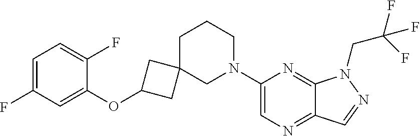 [SMILES: Fc1ccc(F)c(OC2CC3(CCCN(c4cnc5cnn(CC(F)(F)F)c5n4)C3)C2)c1]

Followed the general procedure A using 2-(2,5-difluorophenoxy)-6-azaspiro[3.5]nonane (21.4 mg, 84.5 μmol, 1.0 equiv) and 6-chloro-1-(2,2-difluoroethyl)-1H-pyrazolo[3,4-b]pyrazine (20 mg, 84.5 μmol, 1.0 equiv.) as the starting materials to give 2-(2,5-difluorophenoxy)-6-[1-(2,2,2-trifluoroethyl)-1H-pyrazolo[3,4-b]pyrazin-6-yl]-6-azaspiro[3.5]nonane (19 mg, 50%, two isomers in a ratio of 1:2) as a colorless oil. ¹H NMR (major isomer, 500 MHz, DMSO-d₆) δ 8.58 (s, 1H), 8.17 (s, 1H), 7.22-7.27 (m, 1H), 6.91-6.85 (m, 2H), 6.78-6.69 (m, 1H), 5.18 (q, J=9.1 Hz, 2H), 5.04 (p, J=6.8 Hz, 1H), 3.81 (s, 2H), 3.71 (dq, J=6.1, 3.0 Hz, 2H), 2.45-2.38 (m, 2H), 1.84-1.77 (m, 2H), 1.72-1.70 (m, 2H), 1.66-1.53 (m, 2H). MS m/z: 454 [M+H]⁺.

### 6-[1-(2,2-difluoroethyl)-1H-pyrazolo[3,4-b]pyrazin-6-yl]-2-(3,5-difluorophenoxy)-6-azaspiro[3.5]nonane (31)

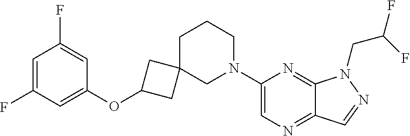 [SMILES: Fc1cc(F)cc(OC2CC3(CCCN(c4cnc5cnn(CC(F)F)c5n4)C3)C2)c1]

Step 1: tert-butyl 2-(3,5-difluorophenoxy)-6-azaspiro[3.5]nonane-6-carboxylate: Followed the general procedure C using 3,5-difluorophenol (53.9 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-(2,5-difluorophenoxy)-6-azaspiro[3.5]nonane-6-carboxylate (74 mg, 51%) as a colorless solid. MS m/z: 354 [M+H]⁺.

Step 2: 2-(3,5-difluorophenoxy)-6-azaspiro[3.5]nonane hydrochloride: Followed the general procedure B tert-butyl 2-(3,5-difluorophenoxy)-6-azaspiro[3.5]nonane-6-carboxylate (74 mg) as the starting material to give the crude product 2-(2,5-difluorophenoxy)-6-azaspiro[3.5]nonane hydrochloride (55 mg). MS m/z: 254 [M+H]⁺.

Step 3: 6-[1-(2,2-difluoroethyl)-1H-pyrazolo[3,4-b]pyrazin-6-yl]-2-(3,5-difluorophenoxy)-6-azaspiro[3.5]nonane: Followed the general procedure A using 2-(3,5-difluorophenoxy)-6-azaspiro[3.5]nonane hydrochloride (19.9 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-(3,5-difluorophenoxy)-6-azaspiro[3.5]nonane (30 mg, 100%, two isomers in a ratio of 3:10) as a colorless oil. ¹H NMR (major isomer, 500 MHz, DMSO-d₆) δ 8.56 (s, 1H), 8.13 (s, 1H), 6.79-6.74 (m, 1H), 6.60 (dd, J=9.3, 2.3 Hz, 2H), 6.44 (tt, J=54.9, 3.8 Hz, 1H), 5.00 (p, J=6.9 Hz, 1H), 4.71 (td, J=15.1, 3.8 Hz, 2H), 3.78 (s, 2H), 3.70 (t, J=5.5 Hz, 2H), 2.45-2.36 (m, 2H), 1.76 (ddd, J=12.4, 6.1, 2.8 Hz, 2H), 1.69 (dd, J=7.5, 4.4 Hz, 2H), 1.57 (dt, J=7.1, 3.7 Hz, 2H). MS m/z: 436 [M+H]⁺.

### 6-[1-(2,2-difluoroethyl)-1H-pyrazolo[3,4-b]pyrazin-6-yl]-2-{[3-(trifluoromethyl)pyridin-2-yl]oxy}-6-azaspiro[3.5]nonane (32)

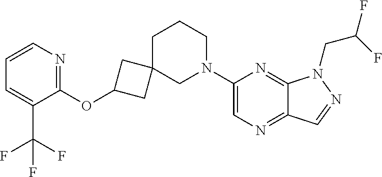 [SMILES: FC(F)Cn1ncc2ncc(N3CCCC4(CC(Oc5ncccc5C(F)(F)F)C4)C3)nc21]

Step 1: tert-butyl 2-{[3-(trifluoromethyl)pyridin-2-yl]oxy}-6-azaspiro[3.5]nonane-6-carboxylate: Followed the general procedure E using 2-bromo-3-(trifluoromethyl)pyridine (93.6 mg, 414 μmol, 1 equiv.) and tert-butyl 2-hydroxy-6-azaspiro[3.5]nonane-6-carboxylate (100 mg, 414 μmol, 1.0 equiv.) as the starting materials to give tert-butyl 2-{[3-(trifluoromethyl)pyridin-2-yl]oxy}-6-azaspiro[3.5]nonane-6-carboxylate (128 mg, 80%) as a colorless oil. MS m/z: 387 [M+H]⁺.

Step 2: tert-butyl 2-{[3-(trifluoromethyl)pyridin-2-yl]oxy}-6-azaspiro[3.5]nonane hydrochloride: Followed the general procedure B using tert-butyl 2-{[3-(trifluoromethyl)pyridin-2-yl]oxy}-6-azaspiro[3.5]nonane-6-carboxylate (127 mg) as the starting material to give the crude product 2-{[3-(trifluoromethyl)pyridin-2-yl]oxy}-6-azaspiro[3.5]nonane hydrochloride (100 mg). MS m/z: 286 [M+H]⁺.

Step 3: 6-[1-(2,2-difluoroethyl)-1H-pyrazolo[3,4-b]pyrazin-6-yl]-2-{[3-(trifluoromethyl)pyridin-2-yl]oxy}-6-azaspiro[3.5]nonane: Followed the general procedure A using 2-{[3-(trifluoromethyl)pyridin-2-yl]oxy}-6-azaspiro[3.5]nonane hydrochloride (22.1 mg, 68.6 μmol, 1.0 equiv) and 6-chloro-1-(2,2-difluoroethyl)-1H-pyrazolo[3,4-b]pyrazine (15 mg, 68.6 μmol, 1.0 equiv.) as the starting materials to give 6-[1-(2,2-difluoroethyl)-1H-pyrazolo[3,4-b]pyrazin-6-yl]-2-{[4-(trifluoromethyl)pyridin-2-yl]oxy}-6-azaspiro[3.5]nonane (16 mg, 50%, two isomers in a ratio of 2:3) as a light-yellow oil. ¹H NMR (major isomer, 500 MHz, DMSO-d₆) δ 8.46 (s, 1H), 8.40-8.36 (m, 1H), 8.11-8.06 (m, 1H), 8.09 (s, 1H), 7.18-7.13 (m, 1H), 6.36 (tt, J=55.0, 3.9 Hz, 1H), 5.30 (tt, J=7.2, 5.9 Hz, 1H), 4.62 (td, J=14.9, 3.9 Hz, 2H), 3.79 (s, 2H), 3.69 (dd, J=6.6, 4.2 Hz, 2H), 2.35-2.27 (m, 2H), 1.99-1.92 (m, 2H), 1.76-1.73 (m, 2H), 1.65-1.61 (m, 2H). MS m/z: 469 [M+H]⁺.

### 2-(3,5-difluorophenoxy)-6-[1-(2,2,2-trifluoroethyl)-1H-pyrazolo[3,4-b]pyrazin-6-yl]-6-azaspiro[3.5]nonane (33)

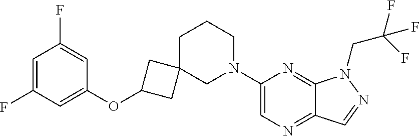 [SMILES: Fc1cc(F)cc(OC2CC3(CCCN(c4cnc5cnn(CC(F)(F)F)c5n4)C3)C2)c1]

Followed the general procedure A using 2-(3,5-difluorophenoxy)-6-azaspiro[3.5]nonane hydrochloride (24.5 mg, 84.5 μmol, 1.0 equiv) and 6-chloro-1-(2,2,2-trifluoroethyl)-1H-pyrazolo[3,4-b]pyrazine (20 mg, 84.5 μmol, 1.0 equiv.) as the starting materials to give 2-(3,5-difluorophenoxy)-6-[1-(2,2,2-trifluoroethyl)-1H-pyrazolo[3,4-b]pyrazin-6-yl]-6-azaspiro[3.5]nonane (20 mg, 52%, two isomers in a ratio of 1:5) as a colorless oil. ¹H NMR (major isomer, 500 MHz, DMSO-d₆) δ 8.58 (s, 1H), 8.17 (s, 1H), 6.77 (tt, J=9.3, 2.3 Hz, 1H), 6.59 (dt, J=9.4, 2.0 Hz, 3H), 5.18 (q, J=9.1 Hz, 2H), 5.00 (p, J=6.9 Hz, 1H), 3.81 (s, 2H), 3.71 (dd, J=6.6, 4.2 Hz, 2H), 2.44-2.36 (m, 2H), 1.80-1.72 (m, 2H), 1.69 (t, J=6.0 Hz, 2H), 1.60-1.52 (m, 2H). MS m/z: 454 [M+H]⁺.

### 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decane (34)

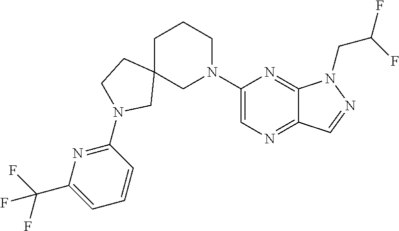 [SMILES: FC(F)Cn1ncc2ncc(N3CCCC4(CCN(c5cccc(C(F)(F)F)n5)C4)C3)nc21]

Step 1: tert-butyl 2-[6-(trifluoromethyl)pyridin-2-yl]-2,7-diazaspiro[4.5]decane-7-carboxylate: Followed the general procedure A tert-butyl 2,7-diazaspiro[4.5]decane-7-carboxylate (200 mg, 832 μmol, 1.0 equiv) and 2-bromo-6-(trifluoromethyl)pyridine (188 mg, 832 μmol, 1.0 equiv.) as the starting materials to give tert-butyl 2-[6-(trifluoromethyl)pyridin-2-yl]-2,7-diazaspiro[4.5]decane-7-carboxylate (260 mg, 81%) as a colorless oil. MS m/z: 386 [M+H]⁺.

Step 2: 2-[6-(trifluoromethyl)pyridin-2-yl]-2,7-diazaspiro[4.5]decane hydrochloride: Followed the general procedure B using tert-butyl 2-[6-(trifluoromethyl)pyridin-2-yl]-2,7-diazaspiro[4.5]decane-7-carboxylate (260 mg) as the starting material to give the crude product 2-[6-(trifluoromethyl)pyridin-2-yl]-2,7-diazaspiro[4.5]decane hydrochloride (250 mg). MS m/z: 286 [M+H]⁺.

Step 3: 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decane: Followed the general procedure A using 2-[6-(trifluoromethyl)pyridin-2-yl]-2,7-diazaspiro[4.5]decane hydrochloride (17.7 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decane (20 mg, 78%) as a white solid. ¹H NMR (500 MHz, DMSO-d₆) δ 8.45 (s, 1H), 8.08 (s, 1H), 7.66 (t, J=7.9 Hz, 1H), 6.94 (d, J=7.2 Hz, 1H), 6.68 (d, J=8.6 Hz, 1H), 6.32 (tt, J=55.0, 3.9 Hz, 1H), 4.50 (tt, J=14.8, 3.7 Hz, 2H), 3.75 (dd, J=22.9, 14.3 Hz, 4H), 3.67-3.57 (m, 1H), 3.54 (d, J=9.8 Hz, 1H), 3.45 (d, J=11.0 Hz, 1H), 3.14 (d, J=10.9 Hz, 1H), 1.96-1.91 (m, 1H), 1.84-1.62 (m, 5H). MS m/z: 468 [M+H]⁺.

### 6-[1-(2,2-difluoroethyl)-1H-pyrazolo[3,4-b]pyrazin-6-yl]-2-[3-(trifluoromethyl)pyridin-4-yl]-2,6-diazaspiro[3.5]nonane (35)

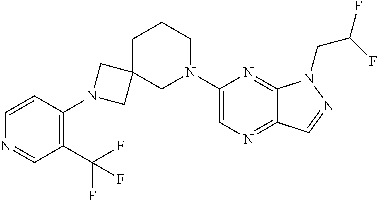 [SMILES: FC(F)Cn1ncc2ncc(N3CCCC4(C3)CN(c3ccncc3C(F)(F)F)C4)nc21]

Step 1: tert-butyl 2-[3-(trifluoromethyl)pyridin-4-yl]-2,6-diazaspiro[3.5]nonane-6-carboxylate: Followed the general procedure D using tert-butyl 2,6-diazaspiro[3.5]nonane-6-carboxylate (50 mg, 221 μmol, 1.0 equiv) and 4-bromo-3-(trifluoromethyl)pyridine (50 mg, 221 μmol, 1.0 equiv.) as the starting materials to give tert-butyl 2-[3-(trifluoromethyl)pyridin-4-yl]-2,6-diazaspiro[3.5]nonane-6-carboxylate (80 mg, 98%) as a white solid. MS m/z: 372 [M+H]⁺.

Step 2: 2-[3-(trifluoromethyl)pyridin-4-yl]-2,6-diazaspiro[3.5]nonane hydrochloride: Followed the general procedure B using tert-butyl 2-[3-(trifluoromethyl)pyridin-4-yl]-2,6-diazaspiro[3.5]nonane-6-carboxylate (80 mg) as the starting material to give the crude product 2-[2-(trifluoromethyl)pyridin-3-yl]-2,6-diazaspiro[3.5]nonane hydrochloride (70 mg). MS m/z: 272 [M+H]⁺.

Step 3: 6-[1-(2,2-difluoroethyl)-1H-pyrazolo[3,4-b]pyrazin-6-yl]-2-[3-(trifluoromethyl)pyridin-4-yl]-2,6-diazaspiro[3.5]nonane: Followed the general procedure A using 2-[3-(trifluoromethyl)pyridin-4-yl]-2,6-diazaspiro[3.5]nonanehydrochloride (70 mg, 227 μmol, 1.0 equiv) and 6-chloro-1-(2,2-difluoroethyl)-1H-pyrazolo[3,4-b]pyrazine (50 mg, 227 μmol, 1.0 equiv) as the starting materials to give 6-[1-(2,2-difluoroethyl)-1H-pyrazolo[3,4-b]pyrazin-6-yl]-2-[3-(trifluoromethyl)pyridin-4-yl]-2,6-diazaspiro[3.5]nonane (62 mg, 60%) as a white solid. ¹H NMR (500 MHz, DMSO-d₆) δ 8.56 (s, 1H), 8.21 (d, J=5.7 Hz, 1H), 8.13 (s, 1H), 6.73 (d, J=2.3 Hz, 1H), 6.54 (dd, J=5.7, 2.3 Hz, 1H), 6.40 (tt, J=54.9, 3.7 Hz, 1H), 4.67 (td, J=15.1, 3.8 Hz, 2H), 3.98 (s, 2H), 3.80 (d, J=8.2 Hz, 2H), 3.74 (t, J=5.6 Hz, 2H), 3.70 (d, J=8.2 Hz, 2H), 1.96-1.88 (m, 2H), 1.65 (tt, J=8.2, 4.1 Hz, 2H). MS m/z: 454 [M+H]⁺.

### 2-[1-(2,2-difluoroethyl)-1H-pyrazolo[3,4-b]pyrazin-6-yl]-7-[6-(trifluoromethyl)pyridin-2-yl]-2,7-diazaspiro[4.5]decane (36)

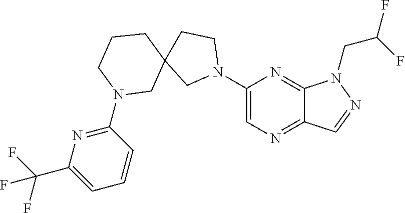 [SMILES: FC(F)Cn1ncc2ncc(N3CCC4(CCCN(c5cccc(C(F)(F)F)n5)C4)C3)nc21]

Step 1: tert-butyl 7-[6-(trifluoromethyl)pyridin-2-yl]-2,7-diazaspiro[4.5]decane-2-carboxylate: Followed the general procedure A using tert-butyl 2,7-diazaspiro[4.5]decane-2-carboxylate (200 mg, 832 μmol, 1.0 equiv) and 2-bromo-6-(trifluoromethyl)pyridine (188 mg, 832 μmol, 1.0 equiv.) as the starting materials to give tert-butyl 7-[6-(trifluoromethyl)pyridin-2-yl]-2,7-diazaspiro[4.5]decane-2-carboxylate (162 mg, 51%) as a colorless oil. MS m/z: 386 [M+H]⁺.

Step 2: 7-[6-(trifluoromethyl)pyridin-2-yl]-2,7-diazaspiro[4.5]decane hydrochloride: Followed the general procedure B using tert-butyl 7-[6-(trifluoromethyl)pyridin-2-yl]-2,7-diazaspiro[4.5]decane-2-carboxylate (162 mg) as the starting material to give the crude product 7-[6-(trifluoromethyl)pyridin-2-yl]-2,7-diazaspiro[4.5]decane hydrochloride (150 mg). MS m/z: 286 [M+H]⁺.

Step 3: 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decane: Followed the general procedure A using 7-[6-(trifluoromethyl)pyridin-2-yl]-2,7-diazaspiro[4.5]decane hydrochloride (17.7 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 2-[1-(2,2-difluoroethyl)-1H-pyrazolo[3,4-b]pyrazin-6-yl]-7-[6-(trifluoromethyl)pyridin-2-yl]-2,7-diazaspiro[4.5]decane (21 mg, 82%) as a white solid. ¹H NMR (500 MHz, DMSO-d₆) δ 8.11 (s, 1H), 8.05 (s, 1H), 7.65 (dd, J=8.7, 7.3 Hz, 1H), 7.13 (d, J=8.8 Hz, 1H), 6.92 (d, J=7.2 Hz, 1H), 6.40 (t, J=54.9 Hz, 1H), 4.64 (td, J=15.1, 3.8 Hz, 2H), 3.71 (dd, J=8.7, 5.5 Hz, 2H), 3.66-3.59 (m, 3H), 3.55 (t, J=11.5 Hz, 2H), 3.28 (d, J=11.1 Hz, 1H), 1.98-1.88 (m, 1H), 1.77 (dt, J=12.0, 5.9 Hz, 2H), 1.73-1.60 (m, 3H). MS m/z: 468 [M+H]⁺.

### 6-[1-(2,2-difluoroethyl)-1H-pyrazolo[3,4-b]pyrazin-6-yl]-2-[6-(trifluoromethyl)pyridin-3-yl]-2,6-diazaspiro[3.5]nonane (37)

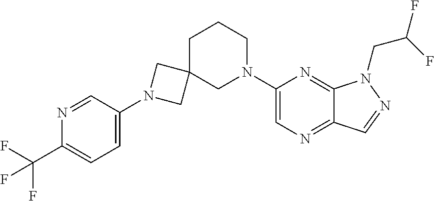 [SMILES: FC(F)Cn1ncc2ncc(N3CCCC4(CN(c5ccc(C(F)(F)F)nc5)C4)C3)nc21]

Step 1: tert-butyl 2-[6-(trifluoromethyl)pyridin-3-yl]-2,6-diazaspiro[3.5]nonane-6-carboxylate: Followed the general procedure D using tert-butyl 2,6-diazaspiro[3.5]nonane-6-carboxylate (50 mg, 221 μmol, 1.0 equiv) and 5-bromo-2-(trifluoromethyl)pyridine (50 mg, 221 μmol, 1.0 equiv.) as the starting materials to give tert-butyl 2-[6-(trifluoromethyl)pyridin-3-yl]-2,6-diazaspiro[3.5]nonane-6-carboxylate (52 mg, 63%) as a colorless oil. MS m/z: 372 [M+H]⁺.

Step 2: 2-[6-(trifluoromethyl)pyridin-3-yl]-2,6-diazaspiro[3.5]nonane hydrochloride: Followed the general procedure B using tert-butyl 2-[6-(trifluoromethyl)pyridin-3-yl]-2,6-diazaspiro[3.5]nonane-6-carboxylate (52 mg) as the starting material to give the crude product 2-[6-(trifluoromethyl)pyridin-3-yl]-2,6-diazaspiro[3.5]nonane hydrochloride (52 mg). MS m/z: 272 [M+H]⁺.

Step 3: 6-[1-(2,2-difluoroethyl)-1H-pyrazolo[3,4-b]pyrazin-6-yl]-2-[6-(trifluoromethyl)pyridin-3-yl]-2,6-diazaspiro[3.5]nonane: Followed the general procedure A using 2-[6-(trifluoromethyl)pyridin-3-yl]-2,6-diazaspiro[3.5]nonane hydrochloride (52 mg, 169 μmol, 1.0 equiv) and 6-chloro-1-(2,2-difluoroethyl)-1H-pyrazolo[3,4-b]pyrazine (37 mg, 169 μmol, 1.0 equiv) as the starting materials to give 6-[1-(2,2-difluoroethyl)-1H-pyrazolo[3,4-b]pyrazin-6-yl]-2-[6-(trifluoromethyl)pyridin-3-yl]-2,6-diazaspiro[3.5]nonane (11 mg, 14%) as a white solid. ¹H NMR (500 MHz, DMSO-d₆) δ 8.56 (s, 1H), 8.13 (s, 1H), 7.89 (d, J=2.8 Hz, 1H), 7.58 (d, J=8.5 Hz, 1H), 6.90 (dd, J=8.6, 2.8 Hz, 1H), 6.41 (tt, J=54.9, 3.8 Hz, 1H), 4.67 (td, J=15.0, 3.8 Hz, 2H), 3.99 (s, 2H), 3.80 (d, J=7.8 Hz, 2H), 3.74 (t, J=5.6 Hz, 2H), 3.69 (d, J=7.8 Hz, 2H), 1.93 (t, J=6.0 Hz, 2H), 1.66 (dq, J=10.7, 3.6 Hz, 2H). MS m/z: 454 [M+H]⁺.

### 6-[1-(2,2-difluoroethyl)-1H-pyrazolo[3,4-b]pyrazin-6-yl]-2-[4-(trifluoromethyl)pyridin-2-yl]-2,6-diazaspiro[3.5]nonane (38)

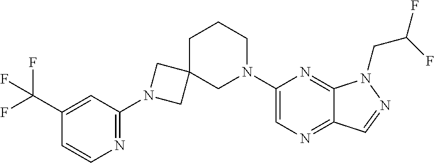 [SMILES: FC(F)Cn1ncc2ncc(N3CCCC4(CN(c5cc(C(F)(F)F)ccn5)C4)C3)nc21]

Followed the general procedure A using 6-[1-(2,2-difluoroethyl)-1H-pyrazolo[3,4-b]pyrazin-6-yl]-2,6-diazaspiro[3.5]nonane hydrochloride (20 mg, 58 μmol, 1.0 equiv) and 2-bromo-4-(trifluoromethyl)pyridine (13 mg, 58 μmol, 1.0 equiv) as the starting materials to give 6-[1-(2,2-difluoroethyl)-1H-pyrazolo[3,4-b]pyrazin-6-yl]-2-[4-(trifluoromethyl)pyridin-2-yl]-2,6-diazaspiro[3.5]nonane (14 mg, 53%) as a white solid. ¹H NMR (500 MHz, DMSO-d₆) δ 8.56 (s, 1H), 8.27 (d, J=5.2 Hz, 1H), 8.13 (s, 1H), 6.85 (dd, J=5.3, 1.6 Hz, 1H), 6.66-6.59 (m, 1H), 6.40 (tt, J=54.9, 3.8 Hz, 1H), 4.67 (td, J=15.1, 3.8 Hz, 2H), 3.98 (s, 2H), 3.81 (d, J=8.2 Hz, 2H), 3.74 (t, J=5.5 Hz, 2H), 3.71 (d, J=8.2 Hz, 2H), 1.91 (dd, J=8.0, 4.1 Hz, 2H), 1.66 (p, J=5.7, 5.3 Hz, 2H). MS m/z: 454 [M+H]⁺.

### 2-[1-(2,2-difluoroethyl)-1H-pyrazolo[3,4-b]pyrazin-6-yl]-6-[3-(trifluoromethyl)pyridin-2-yl]-2,6-diazaspiro[3.5]nonane (39)

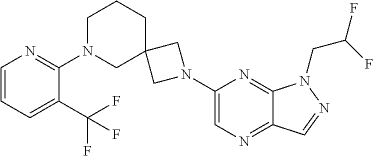 [SMILES: FC(F)Cn1ncc2ncc(N3CC4(CCCN(c5ncccc5C(F)(F)F)C4)C3)nc21]

Followed the general procedure A using 6-[1-(2,2-difluoroethyl)-1H-pyrazolo[3,4-b]pyrazin-6-yl]-2,6-diazaspiro[3.5]nonane hydrochloride (75 mg, 218 μmol, 1.0 equiv) and 2-bromo-3-(trifluoromethyl)pyridine (49 mg, 218 μmol, 1.0 equiv) as the starting materials to give 2-[1-(2,2-difluoroethyl)-1H-pyrazolo[3,4-b]pyrazin-6-yl]-6-[3-(trifluoromethyl)pyridin-2-yl]-2,6-diazaspiro[3.5]nonane (8 mg, 8%) as a white solid. ¹H NMR (500 MHz, DMSO-d₆) δ 8.56 (dd, J=4.9, 1.8 Hz, 1H), 8.12 (s, 1H), 8.09 (dd, J=7.8, 1.9 Hz, 1H), 7.92 (s, 1H), 7.28-7.19 (m, 1H), 6.42 (tt, J=54.9, 3.8 Hz, 1H), 4.66 (td, J=15.0, 3.8 Hz, 2H), 3.92 (q, J=8.7 Hz, 4H), 3.02 (t, J=5.2 Hz, 2H), 1.84 (t, J=5.9 Hz, 2H), 1.68 (dd, J=8.2, 3.9 Hz, 2H). MS m/z: 454 [M+H]⁺.

### 6-(1H-indole-6-carbonyl)-2-{[4-(trifluoromethyl)pyridin-3-yl]oxy}-6-azaspiro[3.5]nonane (40)

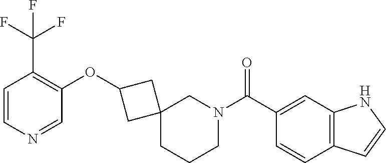 [SMILES: O=C(c1ccc2cc[nH]c2c1)N1CCCC2(CC(Oc3cnccc3C(F)(F)F)C2)C1]

Followed the general procedure G using 1H-indole-6-carboxylic acid (12 mg, 77.5 μmo, 1 equiv.) and 2-{[4-(trifluoromethyl)pyridin-3-yl]oxy}-6-azaspiro[3.5]nonane hydrochloride (25 mg, 77.5 μmol, 1 equiv.) as the starting materials to give 6-(1H-indole-6-carbonyl)-2-{[4-(trifluoromethyl)pyridin-3-yl]oxy}-6-azaspiro[3.5]nonane (25 mg, 75%, two isomers in a ratio of 1:5) as a white solid. ¹H NMR (major isomer, 500 MHz, DMSO-d₆) δ 11.27 (s, 1H), 8.39 (d, J=4.9 Hz, 2H), 7.64 (d, J=4.8 Hz, 1H), 7.59 (d, J=8.1 Hz, 1H), 7.45 (t, J=2.8 Hz, 1H), 7.43 (s, 1H), 7.02 (dd, J=8.1, 1.4 Hz, 1H), 6.47 (ddd, J=3.0, 1.9, 0.9 Hz, 1H), 5.03 (br s, 1H), 3.56 (s, 4H), 1.86-1.63 (m, 5H), 1.50 (d, J=23.4 Hz, 3H). MS m/z: 430 [M+H]⁺.

### 6-[1-(2,2-difluoroethyl)-1H-pyrazolo[3,4-b]pyrazin-6-yl]-2-[5-(trifluoromethyl)pyridin-2-yl]-2,6-diazaspiro[3.5]nonane (41)

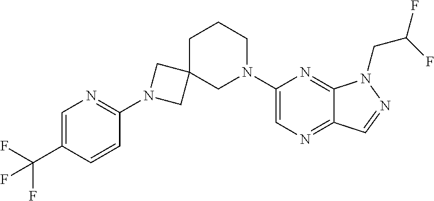 [SMILES: FC(F)Cn1ncc2ncc(N3CCCC4(CN(c5ccc(C(F)(F)F)cn5)C4)C3)nc21]

Followed the general procedure A using 6-[1-(2,2-difluoroethyl)-1H-pyrazolo[3,4-b]pyrazin-6-yl]-2,6-diazaspiro[3.5]nonane hydrochloride (25 mg, 73 μmol, 1.0 equiv) and 2-bromo-5-(trifluoromethyl)pyridine (16.4 mg, 73 μmol, 1.0 equiv) as the starting materials to give 6-[1-(2,2-difluoroethyl)-1H-pyrazolo[3,4-b]pyrazin-6-yl]-2-[5-(trifluoromethyl)pyridin-2-yl]-2,6-diazaspiro[3.5]nonane (19 mg, 58%) as a white solid. ¹H NMR (500 MHz, DMSO-d₆) δ 8.56 (s, 1H), 8.36 (dt, J=2.3, 1.0 Hz, 1H), 8.13 (s, 1H), 7.75 (dd, J=8.9, 2.5 Hz, 1H), 6.47 (d, J=8.9 Hz, 1H), 6.40 (tt, J=54.9, 3.8 Hz, 1H), 4.67 (td, J=15.0, 3.8 Hz, 2H), 3.98 (s, 2H), 3.83 (d, J=8.5 Hz, 2H), 3.73 (d, J=8.3 Hz, 4H), 1.91 (t, J=6.0 Hz, 2H), 1.66 (t, J=6.0 Hz, 2H). MS m/z: 454 [M+H]⁺.

### 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decan-3-one (42)

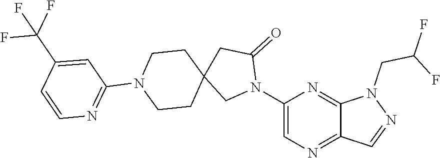 [SMILES: O=C1CC2(CCN(c3cc(C(F)(F)F)ccn3)CC2)CN1c1cnc2cnn(CC(F)F)c2n1]

Step 1: 8-[4-(trifluoromethyl)pyridine-2-yl]-2,8-diazaspiro[4.5]decan-3-one: Followed the general procedure A using 2,8-diazaspiro[4.5]decan-3-one hydrochloride (50 mg, 262 μmol, 1.0 equiv.) and 2-bromo-4-(trifluoromethyl)pyridine (59.3 mg, 262 μmol, 1.0 equiv.) as the starting materials to give 8-[4-(trifluoromethyl)pyridin-2-yl]-2,8-diazaspiro[4.5]decan-3-one (55 mg, 70%) as a off-white solid. MS m/z: 300 [M+H]⁺.

Step 2: 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decan-3-one: Followed the general procedure D using 8-[4-(trifluoromethyl)pyridin-2-yl]-2,8-diazaspiro[4.5]decan-3-one (28.5 mg, 95 μmol, 1.0 equiv.) and 6-chloro-1-(2,2-difluoroethyl)-1H-pyrazolo[3,4-b]pyrazine (25 mg, 95 μmol, 1.0 equiv.) as the starting materials to 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decan-3-one (36 mg, 78%) as an off-white solid. ¹H NMR (500 MHz, DMSO-d₆) δ 9.65 (s, 1H), 8.48 (s, 1H), 8.33 (d, J=5.1 Hz, 1H), 7.13 (d, J=1.6 Hz, 1H), 6.86 (dd, J=5.2, 1.5 Hz, 1H), 6.52 (tt, J=54.7, 3.7 Hz, 1H), 4.87 (td, J=15.1, 3.7 Hz, 2H), 3.97 (s, 2H), 3.93-3.84 (m, 2H), 3.53 (dt, J=13.1, 6.0 Hz, 2H), 2.75 (s, 2H), 1.72 (t, J=5.6 Hz, 4H). MS m/z: 482 [M+H]⁺.

### 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decan-3-one (43)

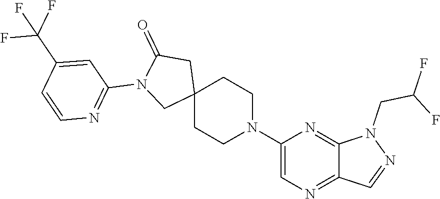 [SMILES: O=C1CC2(CCN(c3cnc4cnn(CC(F)F)c4n3)CC2)CN1c1cc(C(F)(F)F)ccn1]

Step 1: 8-[1-(2,2-difluoroethyl)-1H-pyrazolo[3,4-b]pyrazin-6-yl]-2,8-diazaspiro[4.5]decan-3-one: Followed the general procedure A using 2,8-diazaspiro[4.5]decan-3-one hydrochloride (87 mg, 457 μmol, 1.0 equiv.) and 6-chloro-1-(2,2-difluoroethyl)-1H-pyrazolo[3,4-b]pyrazine (100 mg, 457 μmol, 1.0 equiv.) as the starting materials to give 8-[1-(2,2-difluoroethyl)-1H-pyrazolo[3,4-b]pyrazin-6-yl]-2,8-diazaspiro[4.5]decan-3-one (143 mg, 93%) as a white solid. MS m/z: 337 [M+H]⁺.

Step 2: 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decan-3-one: Followed the general procedure D using 8-[1-(2,2-difluoroethyl)-1H-pyrazolo[3,4-b]pyrazin-6-yl]-2,8-diazaspiro[4.5]decan-3-one (20 mg, 60 μmol, 1.0 equiv.) and 2-bromo-4-(trifluoromethyl)pyridine (16 mg, 71 μmol, 1.2 equiv.) as the starting materials to 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decan-3-one (21 mg, 74%) as a colorless oil. ¹H NMR (500 MHz, DMSO-d₆) δ 8.69-8.64 (m, 2H), 8.67-8.63 (m, 2H), 8.50 (s, 1H), 8.13 (s, 1H), 7.52 (dd, J=4.9, 1.0 Hz, 1H), 6.44 (tt, J=54.9, 3.8 Hz, 2H), 4.69 (td, J=15.0, 3.8 Hz, 2H), 4.01-3.95 (m, 2H), 3.94 (s, 2H), 3.72-3.67 (m, 2H), 2.71 (s, 2H), 1.81-1.67 (m, 4H). MS m/z: 482 [M+H]⁺.

### 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decan-1-one (44)

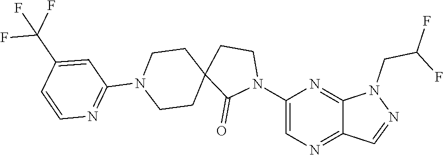 [SMILES: O=C1N(c2cnc3cnn(CC(F)F)c3n2)CCC12CCN(c1cc(C(F)(F)F)ccn1)CC2]

Step 1: 8-[4-(trifluoromethyl)pyridin-2-yl]-2,8-diazaspiro[4.5]decan-1-one: Followed the general procedure A using 2,8-diazaspiro[4.5]decan-1-one hydrochloride (50 mg, 262 μmol, 1.0 equiv.) and 2-bromo-4-(trifluoromethyl)pyridine (59.3 mg, 262 μmol, 1.0 equiv.) as the starting materials to give 8-[4-(trifluoromethyl)pyridin-2-yl]-2,8-diazaspiro[4.5]decan-1-one (55 mg, 70%) as a off-white solid. MS m/z: 300 [M+H]⁺.

Step 2: 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decan-3-one: Followed the general procedure D using 8-[4-(trifluoromethyl)pyridin-2-yl]-2,8-diazaspiro[4.5]decan-1-one (28.5 mg, 95 μmol, 1.0 equiv.) and 6-chloro-1-(2,2-difluoroethyl)-1H-pyrazolo[3,4-b]pyrazine (25 mg, 95 μmol, 1.0 equiv.) as the starting materials to 2-[1-(2,2-difluoroethyl)-1H-pyrazolo[3,4-b]pyrazin-6-yl]-8-[4-(trifluoromethyl)pyridin-2-yl]-2,8-diazaspiro[4.5]decan-1-one (39 mg, 85%) as a yellow oil. ¹H NMR (500 MHz, DMSO-d₆) δ 9.66 (s, 1H), 8.49 (s, 1H), 8.34 (d, J=5.1 Hz, 1H), 7.16 (s, 1H), 6.87 (dd, J=5.2, 1.3 Hz, 1H), 6.52 (tt, J=54.6, 3.6 Hz, 1H), 4.88 (td, J=15.1, 3.6 Hz, 2H), 4.32 (dt, J=13.6, 4.1 Hz, 2H), 4.10 (dd, J=7.9, 6.1 Hz, 2H), 3.22 (ddd, J=13.9, 11.3, 3.0 Hz, 2H), 2.30-2.23 (m, 2H), 1.80 (ddd, J=13.1, 11.3, 4.2 Hz, 2H), 1.74-1.62 (m, 2H). MS m/z: 482 [M+H]⁺.

### 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decan-1-one (45)

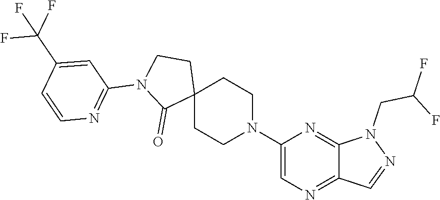 [SMILES: O=C1N(c2cc(C(F)(F)F)ccn2)CCC12CCN(c1cnc3cnn(CC(F)F)c3n1)CC2]

Step 1: 8-[1-(2,2-difluoroethyl)-1H-pyrazolo[3,4-b]pyrazin-6-yl]-2,8-diazaspiro[4.5]decan-1-one: Followed the general procedure A using 2,8-diazaspiro[4.5]decan-1-one hydrochloride (262 mg, 1.37 mmol, 1.0 equiv.) and 6-chloro-1-(2,2-difluoroethyl)-1H-pyrazolo[3,4-b]pyrazine (300 mg, 1.37 mmol, 1.0 equiv.) as the starting materials to give 8-[1-(2,2-difluoroethyl)-1H-pyrazolo[3,4-b]pyrazin-6-yl]-2,8-diazaspiro[4.5]decan-1-one (420 mg, 91%) as a off-white solid. MS m/z: 337 [M+H]⁺.

Step 2: 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decan-1-one: Followed the general procedure D using 8-[1-(2,2-difluoroethyl)-1H-pyrazolo[3,4-b]pyrazin-6-yl]-2,8-diazaspiro[4.5]decan-1-one (25 mg, 74 μmol, 1.0 equiv.) and 2-bromo-4-(trifluoromethyl)pyridine (20 mg, 89 μmol, 1.2 equiv.) as the starting materials to 8-[1-(2,2-difluoroethyl)-1H-pyrazolo[3,4-b]pyrazin-6-yl]-2-[4-(trifluoromethyl)pyridin-2-yl]-2,8-diazaspiro[4.5]decan-1-one (22 mg, 61%) as a light-yellow solid. ¹H NMR (500 MHz, DMSO-d₆) δ 8.70 (d, J=5.3 Hz, 1H), 8.67 (dt, J=1.7, 0.8 Hz, 1H), 8.52 (s, 1H), 8.15 (s, 1H), 7.54 (dd, J=5.4, 1.6 Hz, 1H), 6.45 (tt, J=54.9, 3.8 Hz, 1H), 4.71 (td, J=15.0, 3.8 Hz, 2H), 4.43 (dt, J=13.6, 4.1 Hz, 2H), 4.12-4.03 (m, 2H), 3.41-3.34 (m, 2H), 2.21 (t, J=7.0 Hz, 2H), 1.84 (ddd, J=13.3, 11.2, 4.2 Hz, 2H), 1.77-1.65 (m, 2H). MS m/z: 482 [M+H]⁺.

### 6-(1H-indole-6-carbonyl)-2-{[2-(trifluoromethyl)pyridin-3-yl]oxy}-6-azaspiro[3.5]nonane (46)

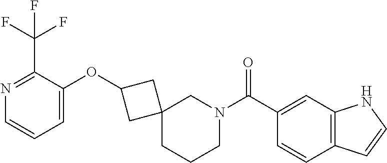 [SMILES: O=C(c1ccc2cc[nH]c2c1)N1CCCC2(CC(Oc3cccnc3C(F)(F)F)C2)C1]

Followed the general procedure G using 1H-indole-6-carboxylic acid (10 mg, 62 μmol, 1 equiv.) and 2-{[2-(trifluoromethyl)pyridin-3-yl]oxy}-6-azaspiro[3.5]nonane hydrochloride (20 mg, 62 μmol, 1 equiv.) as the starting materials to give 6-(1H-indole-6-carbonyl)-2-{[2-(trifluoromethyl)pyridin-3-yl]oxy}-6-azaspiro[3.5]nonane (18 mg, 67%, two isomers in a ratio of 2:3) as a white solid. ¹H NMR (major isomer, 500 MHz, DMSO-d₆) δ 11.22 (s, 1H), 8.23 (d, J=4.5 Hz, 1H), 7.64 (t, J=6.5 Hz, 1H), 7.59-7.54 (m, 1H), 7.51 (d, J=7.0 Hz, 1H), 7.43 (t, J=2.8 Hz, 1H), 7.35 (s, 1H), 6.93 (d, J=8.1 Hz, 1H), 6.45 (t, J=2.6 Hz, 1H), 4.93 (s, 1H), 3.61-3.33 (m, 4H), 2.44-2.29 (m, 2H), 2.06-1.76 (m, 2H), 1.71 (t, J=5.9 Hz, 2H), 1.52 (s, 2H). ¹H NMR (minor isomer, 500 MHz, DMSO-d₆) δ 11.27 (s, 1H), 8.24 (d, J=4.5 Hz, 1H), 7.64 (s, 1H), 7.59 (d, J=8.1 Hz, 1H), 7.56 (br s, 1H), 7.46 (t, J=2.7 Hz, 1H), 7.43 (s, 1H), 7.03 (dd, J=7.5, 5.6 Hz, 1H), 6.47 (ddd, J=3.0, 1.9, 0.9 Hz, 1H), 5.02 (br s, 1H), 3.55-3.38 (m, 4H), 2.47 (s, 2H), 1.77 (d, J=11.8 Hz, 2H), 1.67 (t, J=5.9 Hz, 2H), 1.48 (s, 2H). MS m/z: 430 [M+H]⁺.

### 6-[6-(1,3,4-thiadiazol-2-yl)pyrazin-2-yl]-2-{[2-(trifluoromethyl)pyridin-3-yl]oxy}-6-azaspiro[3.5]nonane (47)

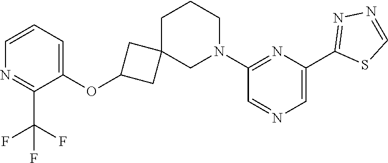 [SMILES: FC(F)(F)c1ncccc1OC1CC2(CCCN(c3cncc(-c4nncs4)n3)C2)C1]

Followed the general procedure A using 2-{[2-(trifluoromethyl)pyridin-3-yl]oxy}-6-azaspiro[3.5]nonane hydrochloride (30 mg, 93 μmol, 1.0 equiv.) and 2-chloro-6-(1,3,4-thiadiazol-2-yl)pyrazine (18.5 mg, 93 μmol, 1.0 equiv.) as the starting materials to give 6-[6-(1,3,4-thiadiazol-2-yl)pyrazin-2-yl]-2-{[2-(trifluoromethyl)pyridin-3-yl]oxy}-6-azaspiro[3.5]nonane (5.3 mg, 13%, two isomers in a ratio of 1:5) as a light-yellow oil. ¹H NMR (major isomer, 500 MHz, DMSO-d₆) δ 9.73 (d, J=4.1 Hz, 1H), 8.62 (d, J=12.0 Hz, 2H), 8.26 (dd, J=4.5, 1.2 Hz, 1H), 7.66 (dd, J=8.5, 4.5 Hz, 1H), 7.60-7.56 (m, 1H), 5.14 (p, J=6.6 Hz, 1H), 3.71 (s, 2H), 3.63 (t, J=5.6 Hz, 2H), 2.47 (td, J=7.4, 3.6 Hz, 2H), 1.85-1.79 (m, 2H), 1.71 (dd, J=7.5, 4.4 Hz, 2H), 1.62-1.51 (m, 2H). MS m/z: 449 [M+H]⁺.

### 6-[1-(2,2,2-trifluoroethyl)-1H-pyrazolo[3,4-b]pyrazin-6-yl]-2-{[2-(trifluoromethyl)pyridin-3-yl]oxy}-6-azaspiro[3.5]nonane (48)

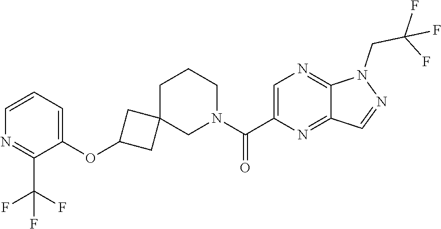 [SMILES: O=C(c1cnc2c(cnn2CC(F)(F)F)n1)N1CCCC2(CC(Oc3cccnc3C(F)(F)F)C2)C1]

Followed the general procedure A using 2-{[2-(trifluoromethyl)pyridin-3-yl]oxy}-6-azaspiro[3.5]nonane hydrochloride (30 mg, 93 μmol, 1.0 equiv.) and 6-chloro-1-(2,2,2-trifluoroethyl)-1H-pyrazolo[3,4-b]pyrazine (22 mg, 93 μmol, 1.0 equiv.) as the starting materials to give 6-[1-(2,2,2-trifluoroethyl)-1H-pyrazolo[3,4-b]pyrazin-6-yl]-2-{[2-(trifluoromethyl)pyridin-3-yl]oxy}-6-azaspiro[3.5]nonane (9 mg, 20%, two isomers in a ratio of 1:5) as a colorless oil. ¹H NMR (major isomer, 500 MHz, DMSO-d₆) δ 8.58 (s, 1H), 8.25 (dd, J=4.5, 1.1 Hz, 1H), 8.17 (s, 1H), 7.66 (dd, J=8.6, 4.5 Hz, 1H), 7.55 (dd, J=8.7, 1.1 Hz, 1H), 5.23-5.12 (m, 3H), 3.81 (s, 2H), 3.71 (t, J=5.5 Hz, 2H), 2.48-2.40 (m, 2H), 1.82-1.75 (m, 2H), 1.70 (d, J=6.4 Hz, 2H), 1.58 (dq, J=7.1, 3.9 Hz, 2H). MS m/z: 449 [M+H]⁺.

### 2-[1-(2,2-difluoroethyl)-1H-pyrazolo[3,4-b]pyrazin-6-yl]-6-[3-(trifluoromethyl)pyridin-4-yl]-2,6-diazaspiro[3.5]nonane (49)

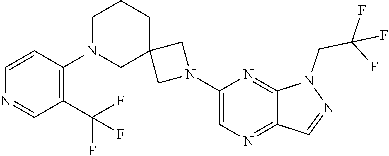 [SMILES: FC(F)(F)Cn1ncc2ncc(N3CC4(CCCN(c5ccncc5C(F)(F)F)C4)C3)nc21]

Step 1: tert-butyl 6-[3-(trifluoromethyl)pyridin-4-yl]-2,6-diazaspiro[3.5]nonane-2-carboxylate: Followed the general procedure D using tert-butyl 2,6-diazaspiro[3.5]nonane-6-carboxylate (50 mg, 221 μmol, 1.0 equiv) and 4-bromo-3-(trifluoromethyl)pyridine (50 mg, 221 μmol, 1.0 equiv.) as the starting materials to give tert-butyl 6-[3-(trifluoromethyl)pyridin-4-yl]-2,6-diazaspiro[3.5]nonane-2-carboxylate (10 mg, 12%) as a colorless oil. MS m/z: 372 [M+H]⁺.

Step 2: 6-[3-(trifluoromethyl)pyridin-4-yl]-2,6-diazaspiro[3.5]nonane hydrochloride: Followed the general procedure B using tert-butyl 6-[3-(trifluoromethyl)pyridin-4-yl]-2,6-diazaspiro[3.5]nonane-2-carboxylate (10 mg) as the starting material to give the crude product 6-[3-(trifluoromethyl)pyridin-4-yl]-2,6-diazaspiro[3.5]nonane hydrochloride (10 mg). MS m/z: 272 [M+H]⁺.

Step 3: 6-[1-(2,2-difluoroethyl)-1H-pyrazolo[3,4-b]pyrazin-6-yl]-2-[6-(trifluoromethyl)pyridin-3-yl]-2,6-diazaspiro[3.5]nonane: Followed the general procedure A using 6-[3-(trifluoromethyl)pyridin-4-yl]-2,6-diazaspiro[3.5]nonane hydrochloride (10 mg, 32.5 μmol, 1.0 equiv) and 6-chloro-1-(2,2-difluoroethyl)-1H-pyrazolo[3,4-b]pyrazine (7.1 mg, 32.5 μmol, 1.0 equiv) as the starting materials to give 2-[1-(2,2-difluoroethyl)-1H-pyrazolo[3,4-b]pyrazin-6-yl]-6-[3-(trifluoromethyl)pyridin-4-yl]-2,6-diazaspiro[3.5]nonane (7 mg, 48%) as a colorless oil. ¹H NMR (500 MHz, DMSO-d₆) δ 8.62 (d, J=5.7 Hz, 1H), 8.12 (s, 1H), 7.93 (s, 1H), 7.31 (d, J=5.7 Hz, 1H), 6.42 (tt, J=54.9, 3.8 Hz, 1H), 4.66 (td, J=15.0, 3.8 Hz, 2H), 3.91 (s, 4H), 3.29 (s, 2H), 3.02 (t, J=5.2 Hz, 2H), 1.85 (t, J=5.9 Hz, 2H), 1.75-1.60 (m, 2H). MS m/z: 454 [M+H]⁺.

### 2-[1-(2,2-difluoroethyl)-1H-pyrazolo[3,4-b]pyrazin-6-yl]-6-[4-(trifluoromethyl)pyridin-3-yl]-2,6-diazaspiro[3.5]nonane (50)

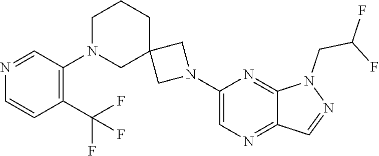 [SMILES: FC(F)Cn1ncc2ncc(N3CC4(CCCN(c5cnccc5C(F)(F)F)C4)C3)nc21]

Step 1: tert-butyl 6-[4-(trifluoromethyl)pyridin-3-yl]-2,6-diazaspiro[3.5]nonane-2-carboxylate: Followed the general procedure D using tert-butyl 2,6-diazaspiro[3.5]nonane-6-carboxylate (50 mg, 221 μmol, 1.0 equiv) and 3-bromo-4-(trifluoromethyl)pyridine (50 mg, 221 μmol, 1.0 equiv.) as the starting materials to give tert-butyl 6-[4-(trifluoromethyl)pyridin-3-yl]-2,6-diazaspiro[3.5]nonane-2-carboxylate (10 mg, 12%) as a colorless oil. MS m/z: 372 [M+H]⁺.

Step 2: 6-[4-(trifluoromethyl)pyridin-3-yl]-2,6-diazaspiro[3.5]nonane hydrochloride: Followed the general procedure B using tert-butyl 6-[4-(trifluoromethyl)pyridin-3-yl]-2,6-diazaspiro[3.5]nonane-2-carboxylate (10 mg) as the starting material to give the crude product 6-[4-(trifluoromethyl)pyridin-3-yl]-2,6-diazaspiro[3.5]nonane hydrochloride (10 mg). MS m/z: 272 [M+H]⁺.

Step 3: 2-[1-(2,2-difluoroethyl)-1H-pyrazolo[3,4-b]pyrazin-6-yl]-6-[4-(trifluoromethyl)pyridin-3-yl]-2,6-diazaspiro[3.5]nonane: Followed the general procedure A using 6-[4-(trifluoromethyl)pyridin-3-yl]-2,6-diazaspiro[3.5]nonane hydrochloride (10 mg, 32.5 μmol, 1.0 equiv) and 6-chloro-1-(2,2-difluoroethyl)-1H-pyrazolo[3,4-b]pyrazine (7.1 mg, 32.5 μmol, 1.0 equiv) as the starting materials to give 2-[1-(2,2-difluoroethyl)-1H-pyrazolo[3,4-b]pyrazin-6-yl]-6-[4-(trifluoromethyl)pyridin-3-yl]-2,6-diazaspiro[3.5]nonane (8 mg, 54%) as a colorless oil. ¹H NMR (500 MHz, DMSO-d₆) δ 8.87 (s, 1H), 8.60 (d, J=5.0 Hz, 1H), 8.12 (s, 1H), 7.92 (s, 1H), 7.67 (d, J=5.0 Hz, 1H), 6.42 (tt, J=54.9, 3.8 Hz, 1H), 4.66 (td, J=15.0, 3.8 Hz, 2H), 3.94 (t, J=7.0 Hz, 4H), 3.22 (s, 2H), 2.88 (t, J=5.2 Hz, 2H), 1.83 (s, 2H), 1.68 (q, J=5.6 Hz, 2H). MS m/z: 454 [M+H]⁺.

### 2-[1-(2,2-difluoroethyl)-1H-pyrazolo[3,4-b]pyrazin-6-yl]-6-[5-(trifluoromethyl)pyridin-3-yl]-2,6-diazaspiro[3.5]nonane (51)

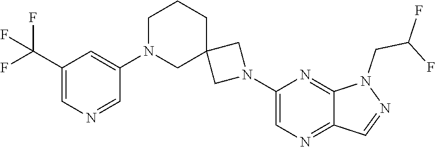 [SMILES: FC(F)Cn1ncc2ncc(N3CC4(CCCN(c5cncc(C(F)(F)F)c5)C4)C3)nc21]

Step 1: tert-butyl 6-[5-(trifluoromethyl)pyridin-3-yl]-2,6-diazaspiro[3.5]nonane-2-carboxylate: Followed the general procedure D using tert-butyl 2,6-diazaspiro[3.5]nonane-6-carboxylate (50 mg, 221 μmol, 1.0 equiv) and 3-bromo-5-(trifluoromethyl)pyridine (50 mg, 221 μmol, 1.0 equiv.) as the starting materials to give tert-butyl 6-[5-(trifluoromethyl)pyridin-3-yl]-2,6-diazaspiro[3.5]nonane-2-carboxylate (65 mg, 79%) as a colorless oil. MS m/z: 372 [M+H]⁺.

Step 2: 6-[5-(trifluoromethyl)pyridin-3-yl]-2,6-diazaspiro[3.5]nonane hydrochloride: Followed the general procedure B using tert-butyl 6-[5-(trifluoromethyl)pyridin-3-yl]-2,6-diazaspiro[3.5]nonane-2-carboxylate (65 mg) as the starting material to give the crude product 6-[5-(trifluoromethyl)pyridin-3-yl]-2,6-diazaspiro[3.5]nonane hydrochloride (50 mg). MS m/z: 272 [M+H]⁺.

Step 3: 2-[1-(2,2-difluoroethyl)-1H-pyrazolo[3,4-b]pyrazin-6-yl]-6-[5-(trifluoromethyl)pyridin-3-yl]-2,6-diazaspiro[3.5]nonane: Followed the general procedure A using 6 6-[5-(trifluoromethyl)pyridin-3-yl]-2,6-diazaspiro[3.5]nonane hydrochloride (30 mg, 97.5 μmol, 1.0 equiv) and 6-chloro-1-(2,2-difluoroethyl)-1H-pyrazolo[3,4-b]pyrazine (21.3 mg, 97.5 μmol, 1.0 equiv) as the starting materials to give 2-[1-(2,2-difluoroethyl)-1H-pyrazolo[3,4-b]pyrazin-6-yl]-6-[5-(trifluoromethyl)pyridin-3-yl]-2,6-diazaspiro[3.5]nonane (16 mg, 36%) as a colorless oil. ¹H NMR (500 MHz, DMSO-d₆) δ 8.65 (d, J=2.8 Hz, 1H), 8.29 (dd, J=1.9, 0.9 Hz, 1H), 8.14 (s, 1H), 7.95 (s, 1H), 7.68 (t, J=2.5 Hz, 1H), 6.43 (tt, J=54.9, 3.8 Hz, 1H), 4.66 (td, J=15.0, 3.8 Hz, 2H), 4.06-3.81 (m, 4H), 3.50 (s, 2H), 3.24 (d, J=6.2 Hz, 2H), 1.85 (t, J=5.9 Hz, 2H), 1.70 (t, J=6.0 Hz, 2H). MS m/z: 454 [M+H]⁺.

### 6-[3-(propan-2-yl)-1H-pyrazole-5-carbonyl]-2-{[4-(trifluoromethyl)pyridin-3-yl]oxy}-6-azaspiro[3.5]nonane (52)

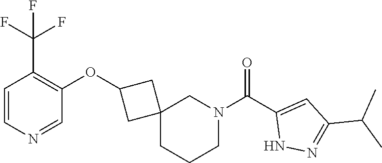 [SMILES: CC(C)c1cc(C(=O)N2CCCC3(CC(Oc4cnccc4C(F)(F)F)C3)C2)[nH]n1]

Followed the general procedure G using 3-(propan-2-yl)-1H-pyrazole-5-carboxylic acid (12 mg, 77.5 μmol, 1 equiv.) and 2-{[4-(trifluoromethyl)pyridin-3-yl]oxy}-6-azaspiro[3.5]nonane hydrochloride (20 mg, 62 μmol, 1 equiv.) as the starting materials to give 6-[3-(propan-2-yl)-1H-pyrazole-5-carbonyl]-2-{[4-(trifluoromethyl)pyridin-3-yl]oxy}-6-azaspiro[3.5]nonane (15 mg, 46%, two isomers in a ratio of 1:4) as a white solid. MS m/z: 423 [M+H]⁺.

### 7-[1-(2,2-difluoroethyl)-1H-pyrazolo[3,4-b]pyrazin-6-yl]-2-phenyl-2,7-diazaspiro[4.5]decane (53)

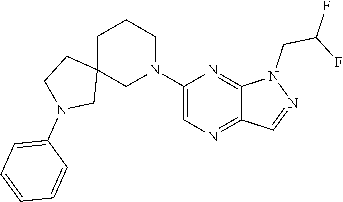 [SMILES: FC(F)Cn1ncc2ncc(N3CCCC4(CCN(c5ccccc5)C4)C3)nc21]

Step 1: tert-butyl 2-phenyl-2,7-diazaspiro[4.5]decane-7-carboxylate: Followed the general procedure F using tert-butyl 2,7-diazaspiro[4.5]decane-7-carboxylate (340 mg, 1.4 mmol, 1 equiv.) and iodobenzene (557 mg, 1.4 mmom, 1 equiv.) as the starting materials to give tert-butyl 2-phenyl-2,7-diazaspiro[4.5]decane-7-carboxylate (320 mg, 72%) as a white solid. MS m/z: 317 [M+H]⁺.

Step 2: 2-phenyl-2,7-diazaspiro[4.5]decane hydrochloride: Followed the general procedure B using tert-butyl 2-phenyl-2,7-diazaspiro[4.5]decane-7-carboxylate (320 mg) as the starting material to give the crude product 2-phenyl-2,7-diazaspiro[4.5]decane hydrochloride (250 mg). MS m/z: 217 [M+H]⁺.

Step 3: 7-[1-(2,2-difluoroethyl)-1H-pyrazolo[3,4-b]pyrazin-6-yl]-2-phenyl-2,7-diazaspiro[4.5]decane: Followed the general procedure A using 2-phenyl-2,7-diazaspiro[4.5]decane hydrochloride (14 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 7-[1-(2,2-difluoroethyl)-1H-pyrazolo[3,4-b]pyrazin-6-yl]-2-phenyl-2,7-diazaspiro[4.5]decane (10 mg, 46%) as a colorless oil. ¹H NMR (500 MHz, DMSO-d₆) δ 8.43 (s, 1H), 8.08 (s, 1H), 7.12 (dd, J=8.6, 7.2 Hz, 2H), 6.59-6.53 (m, 1H), 6.52-6.44 (m, 2H), 6.31 (tt, J=54.9, 3.9 Hz, 1H), 4.48 (td, J=15.1, 3.9 Hz, 2H), 3.86-3.75 (m, 2H), 3.74 (s, 2H), 3.47-3.38 (m, 1H), 3.33-3.26 (m, 1H), 3.19 (d, J=9.5 Hz, 1H), 2.99 (d, J=9.7 Hz, 1H), 1.92 (ddd, J=11.6, 7.3, 3.8 Hz, 1H), 1.82-1.56 (m, 5H). MS m/z: 399 [M+H]⁺.

### 2-[1-(2,2-difluoroethyl)-1H-pyrazolo[3,4-b]pyrazin-6-yl]-7-phenyl-2,7-diazaspiro[4.5]decane (54)

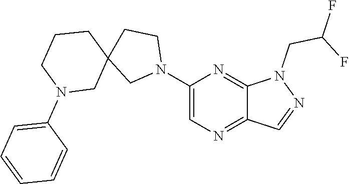 [SMILES: FC(F)Cn1ncc2ncc(N3CCC4(CCCN(c5ccccc5)C4)C3)nc21]

Step 1: tert-butyl 7-phenyl-2,7-diazaspiro[4.5]decane-2-carboxylate: Followed the general procedure F using tert-butyl 2,7-diazaspiro[4.5]decane-2-carboxylate (340 mg, 1.4 mmol, 1 equiv.) and iodobenzene (557 mg, 1.4 mmom, 1 equiv.) as the starting materials to give tert-butyl 7-phenyl-2,7-diazaspiro[4.5]decane-2-carboxylate (170 mg, 38%) as a white solid. MS m/z: 317 [M+H]⁺.

Step 2: 7-phenyl-2,7-diazaspiro[4.5]decane hydrochloride: Followed the general procedure B using tert-butyl 7-phenyl-2,7-diazaspiro[4.5]decane-2-carboxylate (160 mg) as the starting material to give the crude product 7-phenyl-2,7-diazaspiro[4.5]decane hydrochloride (140 mg). MS m/z: 217 [M+H]⁺.

Step 3: 2-[1-(2,2-difluoroethyl)-1H-pyrazolo[3,4-b]pyrazin-6-yl]-7-phenyl-2,7-diazaspiro[4.5]decane: Followed the general procedure A using 7-phenyl-2,7-diazaspiro[4.5]decane hydrochloride (14 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 2-[1-(2,2-difluoroethyl)-1H-pyrazolo[3,4-b]pyrazin-6-yl]-7-phenyl-2,7-diazaspiro[4.5]decane (14 mg, 64%) as a colorless oil. ¹H NMR (500 MHz, DMSO-d₆) δ 8.11 (s, 1H), 8.09 (s, 1H), 7.22-7.09 (m, 2H), 6.99-6.90 (m, 2H), 6.75 (tt, J=7.2, 1.1 Hz, 1H), 6.43 (tt, J=54.9, 3.9 Hz, 1H), 4.67 (td, J=15.0, 3.9 Hz, 2H), 3.72 (t, J=7.1 Hz, 2H), 3.66 (d, J=11.2 Hz, 1H), 3.40 (s, 1H), 3.24-3.18 (m, 1H), 3.11 (d, J=12.0 Hz, 1H), 3.03-2.97 (m, 1H), 2.95 (d, J=12.0 Hz, 1H), 2.11-2.01 (m, 1H), 1.86 (d, J=9.6 Hz, 1H), 1.79-1.64 (m, 3H), 1.60-1.54 (m, 1H). MS m/z: 399 [M+H]⁺.

### 7-[1-(2,2-difluoroethyl)-1H-pyrazolo[3,4-b]pyrazin-6-yl]-2-phenyl-2,7-diazaspiro[3.5]nonane (55)

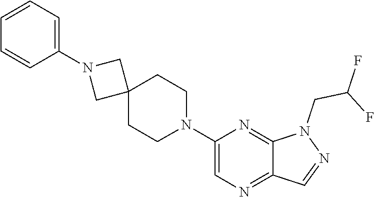 [SMILES: FC(F)Cn1ncc2ncc(N3CCC4(CC3)CN(c3ccccc3)C4)nc21]

Step 1: tert-butyl 2-phenyl-2,7-diazaspiro[3.5]nonane-7-carboxylate: Followed the general procedure F using tert-butyl 2,7-diazaspiro[3.5]nonane-7-carboxylate (320 mg, 1.4 mmol, 1 equiv.) and iodobenzene (557 mg, 1.4 mmom, 1 equiv.) as the starting materials to give tert-butyl 2-phenyl-2,7-diazaspiro[3.5]nonane-7-carboxylate (68 mg, 16%) as a white solid. MS m/z: 303 [M+H]⁺.

Step 2: 7-phenyl-2,7-diazaspiro[3.5]nonane hydrochloride: Followed the general procedure B using tert-butyl 2-phenyl-2,7-diazaspiro[3.5]nonane-7-carboxylate (68 mg) as the starting material to give the crude product 2-phenyl-2,7-diazaspiro[3.5]nonane hydrochloride (50 mg). MS m/z: 203 [M+H]⁺.

Step 3: 7-[1-(2,2-difluoroethyl)-1H-pyrazolo[3,4-b]pyrazin-6-yl]-2-phenyl-2,7-diazaspiro[3.5]nonane: Followed the general procedure A using 2-phenyl-2,7-diazaspiro[3.5]nonane hydrochloride (13.1 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 7-[1-(2,2-difluoroethyl)-1H-pyrazolo[3,4-b]pyrazin-6-yl]-2-phenyl-2,7-diazaspiro[3.5]nonane (10.4 mg, 49%) as a colorless oil. ¹H NMR (500 MHz, DMSO-d₆) δ 8.50 (s, 1H), 8.13 (s, 1H), 7.17 (t, J=7.7 Hz, 2H), 6.66 (t, J=7.3 Hz, 1H), 6.43 (d, J=7.6 Hz, 2H), 6.43 (tt, J=54.9, 3.9 Hz, 1H), 4.70 (td, J=15.0, 3.9 Hz, 2H), 3.82-3.69 (m, 4H), 3.62 (s, 4H), 1.89-1.81 (m, 4H). MS m/z: 385 [M+H]⁺.

### 2-[1-(2,2-difluoroethyl)-1H-pyrazolo[3,4-b]pyrazin-6-yl]-7-phenyl-2,7-diazaspiro[3.5]nonane (56)

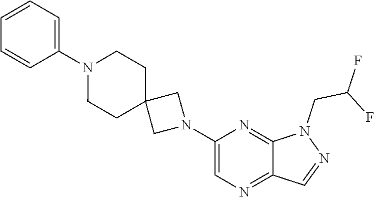 [SMILES: FC(F)Cn1ncc2ncc(N3CC4(CCN(c5ccccc5)CC4)C3)nc21]

Step 1: tert-butyl 7-phenyl-2,7-diazaspiro[3.5]nonane-2-carboxylate: Followed the general procedure F using tert-butyl 2,7-diazaspiro[3.5]nonane-2-carboxylate (320 mg, 1.4 mmol, 1 equiv.) and iodobenzene (557 mg, 1.4 mmol, 1 equiv.) as the starting materials to give tert-butyl 7-phenyl-2,7-diazaspiro[3.5]nonane-2-carboxylate (37 mg, 9%) as a white solid. MS m/z: 303 [M+H]⁺.

Step 2: 7-phenyl-2,7-diazaspiro[3.5]nonane hydrochloride: Followed the general procedure B using tert-butyl 7-phenyl-2,7-diazaspiro[3.5]nonane-2-carboxylate (37 mg) as the starting material to give the crude product 7-phenyl-2,7-diazaspiro[3.5]nonane hydrochloride (30 mg). MS m/z: 203 [M+H]⁺.

Step 3: 2-[1-(2,2-difluoroethyl)-1H-pyrazolo[3,4-b]pyrazin-6-yl]-7-phenyl-2,7-diazaspiro[3.5]nonane: Followed the general procedure A using 7-phenyl-2,7-diazaspiro[3.5]nonane hydrochloride (13.1 mg, 55 μmol, 1.0 equiv) and 6-chloro-1-(2,2-difluoroethyl)-1H-pyrazolo[3,4-b]pyrazine (12 mg, 55 μmol, 1.0 equiv) as the starting materials to give 2-[1-(2,2-difluoroethyl)-1H-pyrazolo[3,4-b]pyrazin-6-yl]-7-phenyl-2,7-diazaspiro[3.5]nonane (8 mg, 38%) as a colorless oil. ¹H NMR (500 MHz, DMSO-d₆) δ 8.14 (s, 1H), 7.93 (s, 1H), 7.26-7.12 (m, 2H), 7.01-6.91 (m, 2H), 6.81-6.67 (m, 1H), 6.44 (tt, J=54.9, 3.8 Hz, 1H), 4.66 (td, J=15.0, 3.8 Hz, 2H), 3.96 (s, 4H), 3.27-3.08 (m, 4H), 1.97-1.79 (m, 4H). MS m/z: 385 [M+H]⁺.

### 2-(2-phenyl-1,3-thiazol-4-yl)-1-{2-phenyl-2,6-diazaspiro[3.5]nonan-6-yl}ethan-1-one (57)

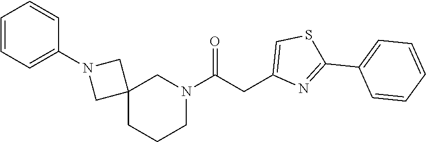 [SMILES: O=C(Cc1csc(-c2ccccc2)n1)N1CCCC2(C1)CN(c1ccccc1)C2]

Followed the general procedure G using 2-phenyl-2,6-diazaspiro[3.5]nonane hydrochloride (16.3 mg, 68.4 μmol, 1 equiv.) and 2-(2-phenyl-1,3-thiazol-4-yl)acetic acid (15 mg, 68.4 μmol, 1 equiv.) as the starting materials to give 2-(2-phenyl-1,3-thiazol-4-yl)-1-{2-phenyl-2,6-diazaspiro[3.5]nonan-6-yl}ethan-1-one (7 mg, 25%) as a white solid. ¹H NMR (500 MHz, DMSO-d₆) δ 7.98-7.93 (m, 1H), 7.92-7.86 (m, 1H), 7.55-7.43 (m, 4H), 7.16-7.10 (m, 2H), 6.67-6.60 (m, 1H), 6.66-6.61 (m, 2H), 3.94 (d, J=9.4 Hz, 2H), 3.75 (s, 1H), 3.68 (s, 1H), 3.56 (t, J=6.8 Hz, 2H), 3.52-3.44 (m, 2H), 3.41 (d, J=7.2 Hz, 1H), 3.31 (d, J=7.2 Hz, 1H), 1.80 (t, J=6.0 Hz, 1H), 1.72 (t, J=6.0 Hz, 1H), 1.47 (s, 1H), 1.37-1.30 (m, 1H). MS m/z: 404 [M+H]⁺.

### 2-(2-phenyl-1,3-thiazol-4-yl)-1-{6-phenyl-2,6-diazaspiro[3.5]nonan-2-yl}ethan-1-one (58)

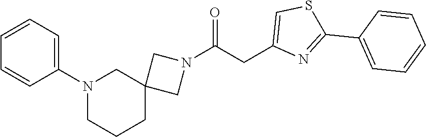 [SMILES: O=C(Cc1csc(-c2ccccc2)n1)N1CC2(CCCN(c3ccccc3)C2)C1]

Step 1: tert-butyl 6-phenyl-2,6-diazaspiro[3.5]nonane-2-carboxylate: Followed the general procedure F using tert-butyl 2,6-diazaspiro[3.5]nonane-2-carboxylate (111 mg, 491 μmol, 1 equiv.) and iodobenzene (200 mg, 982 μmol, 1 equiv.) as the starting materials to give tert-butyl 6-phenyl-2,6-diazaspiro[3.5]nonane-2-carboxylate (34 mg, 23%) as a white solid. MS m/z: 303 [M+H]⁺.

Step 2: 6-phenyl-2,6-diazaspiro[3.5]nonane hydrochloride: Followed the general procedure B using tert-butyl 6-phenyl-2,6-diazaspiro[3.5]nonane-2-carboxylate (34 mg) as the starting material to give the crude product 6-phenyl-2,6-diazaspiro[3.5]nonane hydrochloride (30 mg). MS m/z: 203 [M+H]⁺.

Step 3: 2-(2-phenyl-1,3-thiazol-4-yl)-1-{6-phenyl-2,6-diazaspiro[3.5]nonan-2-yl}ethan-1-one: Followed the general procedure G using 6-phenyl-2,6-diazaspiro[3.5]nonane hydrochloride (16.3 mg, 68.4 μmol, 1 equiv.) and 2-(2-phenyl-1,3-thiazol-4-yl)acetic acid (15 mg, 68.4 μmol, 1 equiv.) as the starting materials to give 2-(2-phenyl-1,3-thiazol-4-yl)-1-{6-phenyl-2,6-diazaspiro[3.5]nonan-2-yl}ethan-1-one (15 mg, 54%) as a white solid. ¹H NMR (500 MHz, DMSO-d₆) δ 7.94-7.87 (m, 2H), 7.50 (s, 1H), 7.49-7.42 (m, 3H), 7.21-7.14 (m, 2H), 7.00-6.94 (m, 2H), 6.83-6.74 (m, 1H), 4.04 (d, J=8.4 Hz, 1H), 3.98 (d, J=8.4 Hz, 1H), 3.67-3.61 (m, 3H), 3.58 (d, J=9.5 Hz, 1H), 3.26 (d, J=11.8 Hz, 1H), 3.13-3.04 (m, 2H), 2.96-2.91 (m, 1H), 1.78-1.74 (m, 1H), 1.69-1.63 (m, 3H). MS m/z: 404 [M+H]⁺.

### 1-{7-benzyl-2,7-diazaspiro[4.5]decan-2-yl}-2-(2-phenyl-1,3-thiazol-4-yl)ethan-1-one (59)

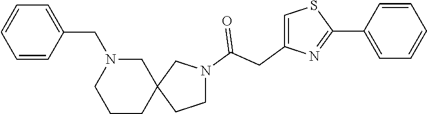 [SMILES: O=C(Cc1csc(-c2ccccc2)n1)N1CCC2(CCCN(Cc3ccccc3)C2)C1]

Followed the general procedure G using 7-benzyl-2,7-diazaspiro[4.5]decane (15.8 mg, 68.4 μmol, 1 equiv.) and 2-(2-phenyl-1,3-thiazol-4-yl)acetic acid (15 mg, 68.4 μmol, 1 equiv.) as the starting materials to give 1-{7-benzyl-2,7-diazaspiro[4.5]decan-2-yl}-2-(2-phenyl-1,3-thiazol-4-yl)ethan-1-one (19 mg, 64%) as a white solid. ¹H NMR (500 MHz, DMSO-d₆) δ 7.93-7.86 (m, 2H), 7.53-7.45 (m, 3H), 7.32-7.17 (m, 6H), 3.77 (d, J=14.4 Hz, 2H), 3.71-3.35 (m, 5H), 2.47-2.01 (m, 4H), 2.00-1.25 (m, 7H). MS m/z: 432 [M+H]⁺.

### 1-{2-benzyl-2,6-diazaspiro[3.5]nonan-6-yl}-2-(2-phenyl-1,3-thiazol-4-yl)ethan-1-one (60)

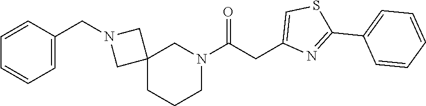 [SMILES: O=C(Cc1csc(-c2ccccc2)n1)N1CCCC2(CN(Cc3ccccc3)C2)C1]

Followed the general procedure G using 2-benzyl-2,6-diazaspiro[3.5]nonane dihydrochloride (33 mg, 114 μmol, 1 equiv.) and 2-(2-phenyl-1,3-thiazol-4-yl)acetic acid (25 mg, 114 μmol, 1 equiv.) as the starting materials to give 1-{2-benzyl-2,6-diazaspiro[3.5]nonan-6-yl}-2-(2-phenyl-1,3-thiazol-4-yl)ethan-1-one (19 mg, 40%) as a white solid. ¹H NMR (500 MHz, DMSO-d₆) δ 7.93-7.84 (m, 2H), 7.49-7.45 (m, 4H), 7.38-7.25 (m, 5H), 3.93 (d, J=39.6 Hz, 4H), 3.69 (d, J=18.9 Hz, 2H), 3.43 (dt, J=26.8, 5.6 Hz, 4H), 1.75 (dt, J=28.0, 6.0 Hz, 2H), 1.48-1.30 (m, 2H), 1.24 (t, J=5.4 Hz, 2H). MS m/z: 418 [M+H]⁺.

### 8-((1H-indol-6-yl)sulfonyl)-2-(6-(trifluoromethyl)pyridin-2-yl)-2,8-diazaspiro[4.5]decan-1-one (61)

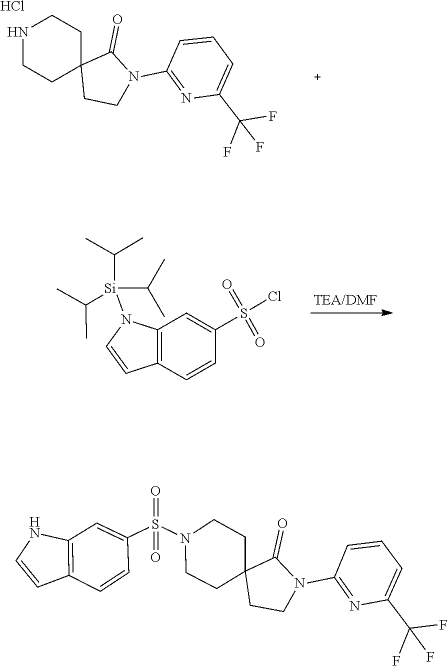 [SMILES: Cl ; O=C1N(c2cccc(C(F)(F)F)n2)CCC12CCNCC2 ; CC(C)[Si](C(C)C)(C(C)C)n1ccc2ccc(S(=O)(=O)Cl)cc21 ; O=C1N(c2cccc(C(F)(F)F)n2)CCC12CCN(S(=O)(=O)c1ccc3cc[nH]c3c1)CC2]

To a stirred solution of 2-[6-(trifluoromethyl)pyridin-2-yl]-2,8-diazaspiro[4.5]decan-1-one (60.0 mg, 0.200 mmol, 1 equiv) and 1-(triisopropylsilyl)indole-6-sulfonyl chloride (82.03 mg, 0.220 mmol, 1.1 equiv) in DMF (1.00 mL) was added TEA (40.57 mg, 0.400 mmol, 2 equiv) at 0° C. The solution was allowed to warm to room temperature and stirred overnight. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 8-((1H-indol-6-yl)sulfonyl)-2-(6-(trifluoromethyl)pyridin-2-yl)-2,8-diazaspiro[4.5]decan-1-one (20 mg, 10.7%) as an white solid. ¹H NMR (400 MHz, DMSO-d₆) δ 11.63 (s, 1H), 8.51 (d, J=8.6 Hz, 1H), 8.05 (t, J=8.0 Hz, 1H), 7.83 (dd, J=1.7, 0.9 Hz, 1H), 7.78 (d, J=8.3 Hz, 1H), 7.68 (t, J=2.8 Hz, 1H), 7.62 (d, J=7.4 Hz, 1H), 7.36 (dd, J=8.3, 1.7 Hz, 1H), 6.62-6.60 (m, J=3.0, 1.9, 0.9 Hz, 1H), 3.86 (t, J=7.0 Hz, 2H), 3.53-3.45 (m, 2H), 2.57 (t, J=10.4 Hz, 2H), 1.88-1.77 (m, 4H), 1.67 (d, J=13.1 Hz, 2H). MS m/z: 479.1 [M+H]⁺.

### 8-(1H-indole-6-carbonyl)-2-(6-(trifluoromethyl)pyridin-2-yl)-2,8-diazaspiro[4.5]decan-1-one (62)

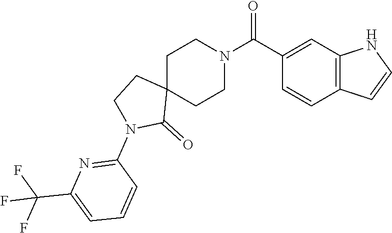 [SMILES: O=C(c1ccc2cc[nH]c2c1)N1CCC2(CC1)CCN(c1cccc(C(F)(F)F)n1)C2=O]

Followed the general procedure G using 1H-indole-6-carboxylic acid (38.8 mg, 0.240 mmol, 1.2 equiv) and 2-[6-(trifluoromethyl)pyridin-2-yl]-2,8-diazaspiro[4.5]decan-1-one (60.0 mg, 0.200 mmol, 1 equiv) as the starting materials to give 8-(1H-indole-6-carbonyl)-2-(6-(trifluoromethyl)pyridin-2-yl)-2,8-diazaspiro[4.5]decan-1-one (10 mg, 10.7%) as a white solid. ¹H NMR (400 MHz, DMSO-d₆) δ 11.28 (s, 1H), 8.60 (d, J=8.5 Hz, 1H), 8.11 (t, J=8.1 Hz, 1H), 7.65 (d, J=7.5 Hz, 1H), 7.59 (d, J=8.1 Hz, 1H), 7.49-7.43 (m, 2H), 7.05 (dd, J=8.2, 1.4 Hz, 1H), 6.48 (s, 1H), 4.00 (t, J=7.1 Hz, 2H), 3.21 (s, 2H), 2.49 (s, 2H), 2.16 (t, J=7.0 Hz, 2H), 1.75 (d, J=11.9 Hz, 2H), 1.65 (s, 2H). MS m/z: 443.1 [M+H]⁺.

### 8-(1-((2-oxaspiro[3.3]heptan-6-yl)methyl)-1H-pyrazolo[3,4-b]pyrazin-6-yl)-2-(6-(trifluoromethyl)pyridin-2-yl)-2,8-diazaspiro[4.5]decan-1-one (63)

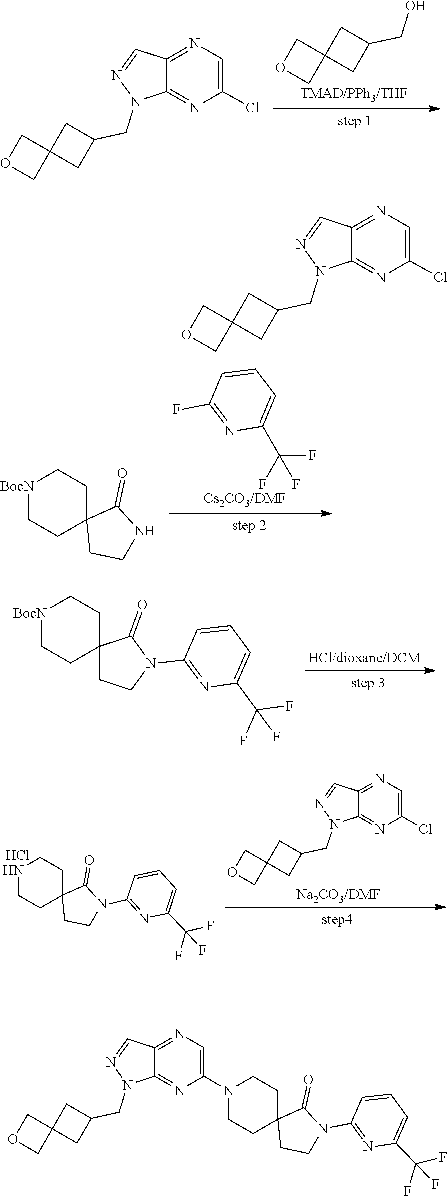

Step 1: 1-((2-oxaspiro[3.3]heptan-6-yl)methyl)-6-chloro-1H-pyrazolo[3,4-b]pyrazine: Followed the general procedure C using 6-chloro-1H-pyrazolo[3,4-b]pyrazine (154.2 mg, 0.998 mmol, 1 equiv) and 2-oxaspiro[3.3]heptan-6-ylmethanol (140.7 mg, 1.098 mmol, 1.1 equiv) as the starting materials to give 6-chloro-1-{2-oxaspiro[3.3]heptan-6-ylmethyl}pyrazolo[3,4-b]pyrazine (134.2 mg, 50.82%) as a yellow oil. LCMS (ES, m/z): 265 [M+H]⁺.

Step 2: tert-butyl 1-oxo-2-(6-(trifluoromethyl)pyridin-2-yl)-2,8-diazaspiro[4.5]decane-8-carboxylate: A mixture of tert-butyl 1-oxo-2,8-diazaspiro[4.5]decane-8-carboxylate (300 mg, 1.180 mmol, 1 equiv), 2-fluoro-6-(trifluoromethyl)pyridine (214.21 mg, 1.298 mmol, 1.1 equiv) and Cs₂CO₃ (768.65 mg, 2.360 mmol, 2 equiv) in DMF (5 mL) was heated at 100° C. overnight. The reaction was monitored by LCMS. The mixture was diluted with water (20 mL), and extracted with EtOAc (20 mL×2). The organic layer was combined, washed with brine, dried, evaporated, and purified with a silica gel column, eluted with gradient of Hexane/EtOAc. The fractions were collected, and concentrated to give tert-butyl 1-oxo-2-(6-(trifluoromethyl)pyridin-2-yl)-2,8-diazaspiro[4.5]decane-8-carboxylate (220 mg, 46%) as a colorless oil. MS m/z: 400 [M−tBu+H]⁺.

Step 3: 2-(6-(trifluoromethyl)pyridin-2-yl)-2,8-diazaspiro[4.5]decan-1-one hydrochloride: Followed the general procedure B using tert-butyl 1-oxo-2-[6-(trifluoromethyl)pyridin-2-yl]-2,8-diazaspiro[4.5]decane-8-carboxylate (220 mg, 0.551 mmol, 1 equiv) as the starting material to give the crude product 2-(6-(trifluoromethyl)pyridin-2-yl)-2,8-diazaspiro[4.5]decan-1-one hydrochloride (210 mg). MS m/z: 300 [M+H]⁺.

Step 4: 8-(1-((2-oxaspiro[3.3]heptan-6-yl)methyl)-1H-pyrazolo[3,4-b]pyrazin-6-yl)-2-(6-(trifluoromethyl)pyridin-2-yl)-2,8-diazaspiro[4.5]decan-1-one: To a stirred solution of 1-((2-oxaspiro[3.3]heptan-6-yl)methyl)-6-chloro-1H-pyrazolo[3,4-b]pyrazine (60 mg, 0.227 mmol, 1 equiv) and 2-[6-(trifluoromethyl)pyridin-2-yl]-2,8-diazaspiro[4.5]decan-1-one hydrochloride (83.5 mg, 0.250 mmol, 1.1 equiv) in DMF (1.00 mL) was added Na₂CO₃ (48.0 mg, 0.454 mmol, 2 equiv) at room temperature. The resulting mixture was stirred for 2 h at 100° C. under nitrogen atmosphere. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fractions were concentrated under vacuum to afford 8-(1-((2-oxaspiro[3.3]heptan-6-yl)methyl)-1H-pyrazolo[3,4-b]pyrazin-6-yl)-2-(6-(trifluoromethyl)pyridin-2-yl)-2,8-diazaspiro[4.5]decan-1-one (20 mg, 16.7%) as a white solid. ¹H NMR (400 MHz, DMSO-d₆) δ 8.60 (d, J=8.6 Hz, 1H), 8.47 (s, 1H), 8.15-8.06 (m, 1H), 8.03 (s, 1H), 7.66 (d, J=7.4 Hz, 1H), 4.51 (s, 2H), 4.43 (d, J=19.5 Hz, 4H), 4.21 (d, J=7.2 Hz, 2H), 4.04 (t, J=7.0 Hz, 2H), 3.36 (d, J=3.0 Hz, 1H), 3.30 (s, 1H), 2.62-2.59 (m, J=15.2, 7.7 Hz, 1H), 2.29-2.18 (m, 4H), 2.06-1.97 (m, 2H), 1.90-1.78 (m, 2H), 1.73 (d, J=13.3 Hz, 2H). MS m/z: 528.2 [M+H]⁺.

### 8-[1-(2,2-difluoroethyl)-1H-pyrazolo[3,4-b]pyrazin-6-yl]-2-[2-(trifluoromethyl)pyridin-3-yl]-2,8-diazaspiro[4.5]decan-3-one (64)

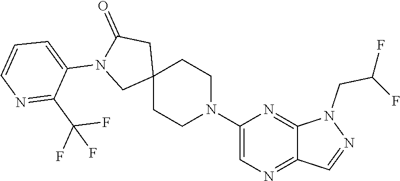 [SMILES: O=C1CC2(CCN(c3cnc4cnn(CC(F)F)c4n3)CC2)CN1c1cccnc1C(F)(F)F]

Step 1: 8-[1-(2,2-difluoroethyl)-1H-pyrazolo[3,4-b]pyrazin-6-yl]-2,8-diazaspiro[4.5]decan-3-one: Followed the general procedure A using 6-chloro-1-(2,2-difluoroethyl)-1H-pyrazolo[3,4-b]pyrazine (100 mg, 457 μmol, 1.0 equiv) and 2,8-diazaspiro[4.5]decan-3-one hydrochloride (87.2 mg, 457 μmol, 1.0 equiv.) as the starting materials to give 8-[1-(2,2-difluoroethyl)-1H-pyrazolo[3,4-b]pyrazin-6-yl]-2,8-diazaspiro[4.5]decan-3-one (143 mg, 93%) as a colorless oil. MS m/z: 337 [M+H]⁺.

Step 2: 8-[1-(2,2-difluoroethyl)-1H-pyrazolo[3,4-b]pyrazin-6-yl]-2-[2-(trifluoromethyl)pyridin-3-yl]-2,8-diazaspiro[4.5]decan-3-one: Followed the general procedure D using 8-[1-(2,2-difluoroethyl)-1H-pyrazolo[3,4-b]pyrazin-6-yl]-2,8-diazaspiro[4.5]decan-3-one (25 mg, 74.3 μmol, 1.0 equiv) and 3-bromo-2-(trifluoromethyl)pyridine (16.8 mg, 74.3 μmol, 1.0 equiv) as the starting materials to give 8-[1-(2,2-difluoroethyl)-1H-pyrazolo[3,4-b]pyrazin-6-yl]-2-[2-(trifluoromethyl)pyridin-3-yl]-2,8-diazaspiro[4.5]decan-3-one (12 mg, 34%) as a white solid. ¹H NMR (500 MHz, DMSO-d₆) δ 8.69 (d, J=5.7 Hz, 1H), 8.51 (s, 1H), 8.27 (d, J=2.1 Hz, 1H), 8.14 (s, 1H), 7.89 (dd, J=5.7, 2.2 Hz, 1H), 6.44 (tt, J=54.9, 3.8 Hz, 1H), 4.70 (td, J=15.1, 3.8 Hz, 2H), 3.95-3.84 (m, 2H), 3.84-3.72 (m, 2H), 3.35-3.30 (m, 2H), 2.71-2.58 (m, 3H), 1.79-1.67 (m, 3H). MS m/z: 482 [M+H]⁺.

### 7-[1-(2,2-difluoroethyl)-1H-pyrazolo[3,4-b]pyrazin-6-yl]-2-[2-(trifluoromethyl)pyridin-3-yl]-2,7-diazaspiro[3.5]nonane (65)

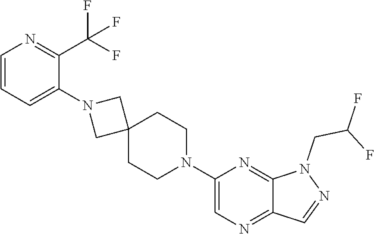 [SMILES: FC(F)Cn1ncc2ncc(N3CCC4(CC3)CN(c3cccnc3C(F)(F)F)C4)nc21]

Step 1: tert-butyl 7-[1-(2,2-difluoroethyl)-1H-pyrazolo[3,4-b]pyrazin-6-yl]-2,7-diazaspiro[3.5]nonane-2-carboxylate: Followed the general procedure A using tert-butyl 2,7-diazaspiro[3.5]nonane-2-carboxylate (200 mg, 884 μmol, 1.0 equiv) and 6-chloro-1-(2,2-difluoroethyl)-1H-pyrazolo[3,4-b]pyrazine (193 mg, 884 μmol, 1.0 equiv.) as the starting materials to give tert-butyl 7-[1-(2,2-difluoroethyl)-1H-pyrazolo[3,4-b]pyrazin-6-yl]-2,7-diazaspiro[3.5]nonane-2-carboxylate (314 mg, 87%) as a white solid. MS m/z: 409 [M+H]⁺.

Step 2: 7-[1-(2,2-difluoroethyl)-1H-pyrazolo[3,4-b]pyrazin-6-yl]-2,7-diazaspiro[3.5]nonane hydrochloride: Followed the general procedure B using tert-butyl 7-[1-(2,2-difluoroethyl)-1H-pyrazolo[3,4-b]pyrazin-6-yl]-2,7-diazaspiro[3.5]nonane-2-carboxylate (314 mg) as the starting material to give the 7-[1-(2,2-difluoroethyl)-1H-pyrazolo[3,4-b]pyrazin-6-yl]-2,7-diazaspiro[3.5]nonane hydrochloride (260 mg). MS m/z: 309 [M+H]⁺.

Step 3: 7-[1-(2,2-difluoroethyl)-1H-pyrazolo[3,4-b]pyrazin-6-yl]-2-[2-(trifluoromethyl)pyridin-3-yl]-2,7-diazaspiro[3.5]nonane: Followed the general procedure A using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 3-fluoro-2-(trifluoromethyl)pyridine (14.4 mg, 87 μmol, 1.0 equiv) as the starting materials to give 7-[1-(2,2-difluoroethyl)-1H-pyrazolo[3,4-b]pyrazin-6-yl]-2-[2-(trifluoromethyl)pyridin-3-yl]-2,7-diazaspiro[3.5]nonane (18 mg, 46%) as a colorless oil. ¹H NMR (500 MHz, DMSO-d₆) δ 8.50 (s, 1H), 8.13 (s, 1H), 7.99 (dd, J=4.4, 1.2 Hz, 1H), 7.48 (dd, J=8.5, 4.3 Hz, 1H), 7.10 (dd, J=8.7, 1.3 Hz, 1H), 6.44 (tt, J=54.9, 3.8 Hz, 1H), 4.70 (td, J=15.0, 3.8 Hz, 2H), 3.85 (d, J=1.6 Hz, 4H), 3.82-3.72 (m, 4H), 1.92-1.76 (m, 4H). MS m/z: 454 [M+H]⁺.

### 7-[1-(2,2-difluoroethyl)-1H-pyrazolo[3,4-b]pyrazin-6-yl]-2-[6-(trifluoromethyl)pyridin-3-yl]-2,7-diazaspiro[3.5]nonane (66)

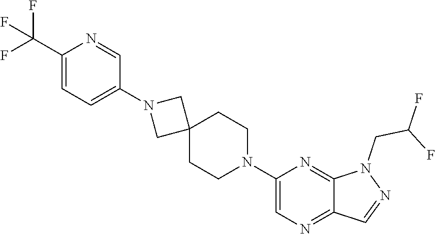 [SMILES: FC(F)Cn1ncc2ncc(N3CCC4(CC3)CN(c3ccc(C(F)(F)F)nc3)C4)nc21]

Followed the general procedure A using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 5-fluoro-2-(trifluoromethyl)pyridine (14.4 mg, 87 μmol, 1.0 equiv) as the starting materials to give 7-[1-(2,2-difluoroethyl)-1H-pyrazolo[3,4-b]pyrazin-6-yl]-2-[6-(trifluoromethyl)pyridin-3-yl]-2,7-diazaspiro[3.5]nonane (20 mg, 51%) as a colorless oil. ¹H NMR (500 MHz, DMSO-d₆) δ 8.51 (s, 1H), 8.14 (s, 1H), 7.91 (d, J=2.7 Hz, 1H), 7.61 (d, J=8.6 Hz, 1H), 6.92 (dd, J=8.6, 2.8 Hz, 1H), 6.44 (tt, J=54.9, 3.8 Hz, 2H), 4.70 (td, J=15.1, 3.8 Hz, 2H), 3.82 (s, 4H), 3.80-3.74 (m, 4H), 1.99-1.77 (m, 4H). MS m/z: 454 [M+H]⁺.

### 7-[1-(2,2-difluoroethyl)-1H-pyrazolo[3,4-b]pyrazin-6-yl]-2-[3-(trifluoromethyl)pyridin-4-yl]-2,7-diazaspiro[3.5]nonane (67)

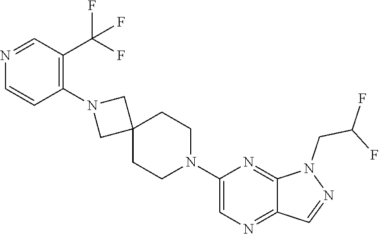 [SMILES: FC(F)Cn1ncc2ncc(N3CCC4(CC3)CN(c3ccncc3C(F)(F)F)C4)nc21]

Followed the general procedure D using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 4-bromo-3-(trifluoromethyl)pyridine hydrobromide (26.7 mg, 87 μmol, 1.0 equiv) as the starting materials to give 7-[1-(2,2-difluoroethyl)-1H-pyrazolo[3,4-b]pyrazin-6-yl]-2-[3-(trifluoromethyl)pyridin-4-yl]-2,7-diazaspiro[3.5]nonane (24 mg, 61%) as a colorless oil. ¹H NMR (500 MHz, DMSO-d₆) δ 8.50 (s, 1H), 8.42 (s, 1H), 8.27 (d, J=6.0 Hz, 1H), 8.13 (s, 1H), 6.48 (d, J=6.0 Hz, 1H), 6.44 (tt, J=54.9, 3.8 Hz, 2H), 4.70 (td, J=15.0, 3.8 Hz, 2H), 3.94 (s, 4H), 3.84-3.67 (m, 4H), 1.93-1.82 (m, 4H). MS m/z: 454 [M+H]⁺.

### 7-[1-(2,2-difluoroethyl)-1H-pyrazolo[3,4-b]pyrazin-6-yl]-2-[2-(trifluoromethyl)pyridin-4-yl]-2,7-diazaspiro[3.5]nonane (68)

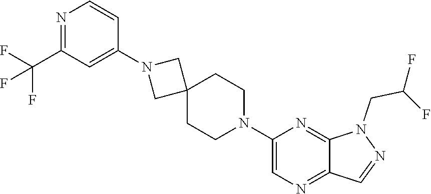 [SMILES: FC(F)Cn1ncc2ncc(N3CCC4(CC3)CN(c3ccnc(C(F)(F)F)c3)C4)nc21]

Followed the general procedure D using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 4-bromo-2-(trifluoromethyl)pyridine (19.7 mg, 87 μmol, 1.0 equiv) as the starting materials to give 7-[1-(2,2-difluoroethyl)-1H-pyrazolo[3,4-b]pyrazin-6-yl]-2-[2-(trifluoromethyl)pyridin-4-yl]-2,7-diazaspiro[3.5]nonane (30 mg, 76%) as a colorless oil. ¹H NMR (500 MHz, DMSO-d₆) δ 8.51 (s, 1H), 8.23 (d, J=5.6 Hz, 1H), 8.14 (s, 1H), 6.73 (d, J=2.2 Hz, 1H), 6.55 (dd, J=5.8, 2.3 Hz, 1H), 6.38 (tt, J=54.9, 3.8 Hz, 1H), 4.70 (td, J=15.0, 3.8 Hz, 2H), 3.82 (s, 4H), 3.80-3.73 (m, 4H), 1.92-1.75 (m, 4H). MS m/z: 454 [M+H]⁺.

### 7-[1-(2,2-difluoroethyl)-1H-pyrazolo[3,4-b]pyrazin-6-yl]-2-[5-(trifluoromethyl)pyridin-3-yl]-2,7-diazaspiro[3.5]nonane (69)

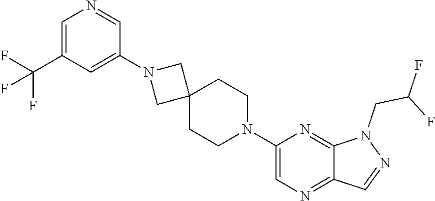 [SMILES: FC(F)Cn1ncc2ncc(N3CCC4(CC3)CN(c3cncc(C(F)(F)F)c3)C4)nc21]

Followed the general procedure D using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 3-bromo-5-(trifluoromethyl)pyridine (19.7 mg, 87 μmol, 1.0 equiv) as the starting materials to give 7-[1-(2,2-difluoroethyl)-1H-pyrazolo[3,4-b]pyrazin-6-yl]-2-[5-(trifluoromethyl)pyridin-3-yl]-2,7-diazaspiro[3.5]nonane (14 mg, 36%) as a colorless oil. ¹H NMR (500 MHz, DMSO-d₆) δ 8.51 (s, 1H), 8.24-8.17 (m, 1H), 8.14 (s, 1H), 8.09 (d, J=2.7 Hz, 1H), 7.07 (t, J=2.4 Hz, 1H), 6.44 (tt, J=54.9, 3.8 Hz, 1H), 4.70 (td, J=15.0, 3.8 Hz, 2H), 3.80 (s, 4H), 3.79-3.71 (m, 4H), 1.93-1.84 (m, 4H). MS m/z: 454 [M+H]⁺.

### 1-benzyl-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (70)

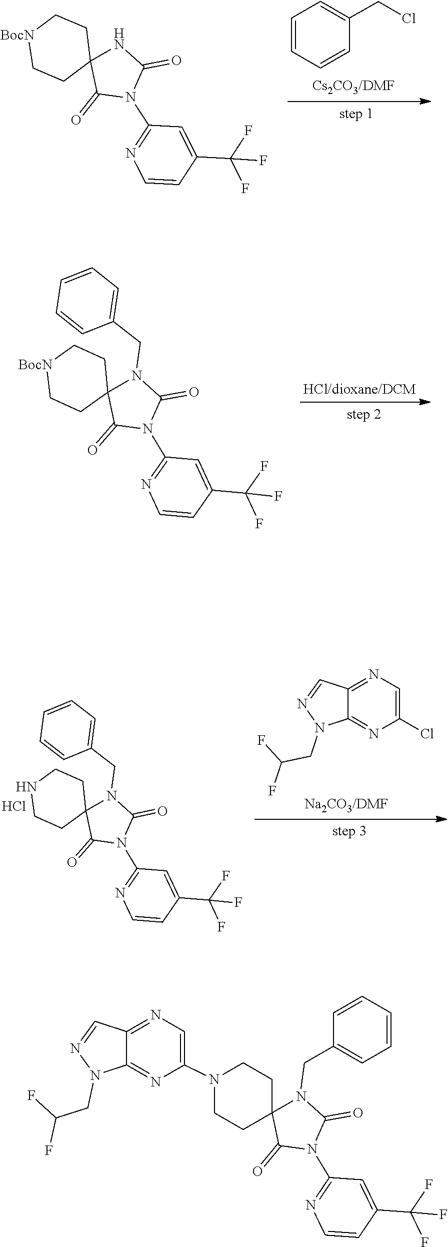

Step 1: tert-butyl 1-benzyl-2,4-dioxo-3-(4-(trifluoromethyl)pyridin-2-yl)-1,3,8-triazaspiro[4.5]decane-8-carboxylate: A solution of tert-butyl 2,4-dioxo-3-(4-(trifluoromethyl)pyridin-2-yl)-1,3,8-triazaspiro[4.5]decane-8-carboxylate (80 mg, 0.193 mmol, 1 equiv), benzyl chloride (26.88 mg, 0.212 mmol, 1.1 equiv) and Cs₂CO₃ (126 mg, 0.386 mmol, 2 equiv) in DMF (1 mL) was stirred for 1.5 h at 50° C. The resulting mixture was diluted with EtOAc (30 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 100% gradient in 30 min; detector, UV 254 nm. This resulted in tert-butyl 1-benzyl-2,4-dioxo-3-(4-(trifluoromethyl)pyridin-2-yl)-1,3,8-triazaspiro[4.5]decane-8-carboxylate (60 mg, 61.60%) as a white solid. MS m/z: 505 [M+H]⁺.

Step 2: 1-benzyl-3-(4-(trifluoromethyl)pyridin-2-yl)-1,3,8-triazaspiro[4.5]decane-2,4-dione hydrochloride: Followed the general procedure B using tert-butyl 1-benzyl-2,4-dioxo-3-(4-(trifluoromethyl)pyridin-2-yl)-1,3,8-triazaspiro[4.5]decane-8-carboxylate (60 mg, 0.119 mmol, 1 equiv) as the starting material to give the crude product 1-benzyl-3-(4-(trifluoromethyl)pyridin-2-yl)-1,3,8-triazaspiro[4.5]decane-2,4-dione hydrochloride (32.6 mg). MS m/z: 405 [M+H]⁺.

Step 3: 1-benzyl-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: A solution of 1-benzyl-3-(4-(trifluoromethyl)pyridin-2-yl)-1,3,8-triazaspiro[4.5]decane-2,4-dione hydrochloride (32.6 mg, 0.074 mmol, 1 equiv) and Na₂CO₃ (15.73 mg, 0.148 mmol, 2 equiv) in DMF (1 mL) was stirred for 2 h at 100° C. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reverse flash chromatography with the following conditions: column, C18 silica gel; mobile phase, MeCN in water, 10% to 100% gradient in 30 min; detector, UV 254 nm. This resulted in 1-benzyl-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (9.0 mg, 20.56%) as a white solid. ¹H NMR (400 MHz, Methanol-d₄) δ 8.87 (d, J=5.2 Hz, 1H), 8.35 (s, 1H), 8.04 (s, 1H), 8.00 (s, 1H), 7.86-7.77 (m, 1H), 7.31 (dd, J=6.7, 2.9 Hz, 2H), 7.24 (dd, J=4.9, 2.0 Hz, 3H), 6.25 (tt, J=55.4, 4.1 Hz, 1H), 4.75-4.62 (m, 4H), 4.57 (d, J=14.1 Hz, 2H), 3.82-3.69 (m, 2H), 2.16-1.97 (m, 4H). MS m/z: 587.1 [M+H]⁺.

### 8-(1-(2,2-difluoroethyl)-1H-pyrazolo[3,4-b]pyrazin-6-yl)-1-(2,2,2-trifluoroethyl)-3-(4-(trifluoromethyl)pyridin-2-yl)-1,3,8-triazaspiro[4.5]decane-2,4-dione (71)

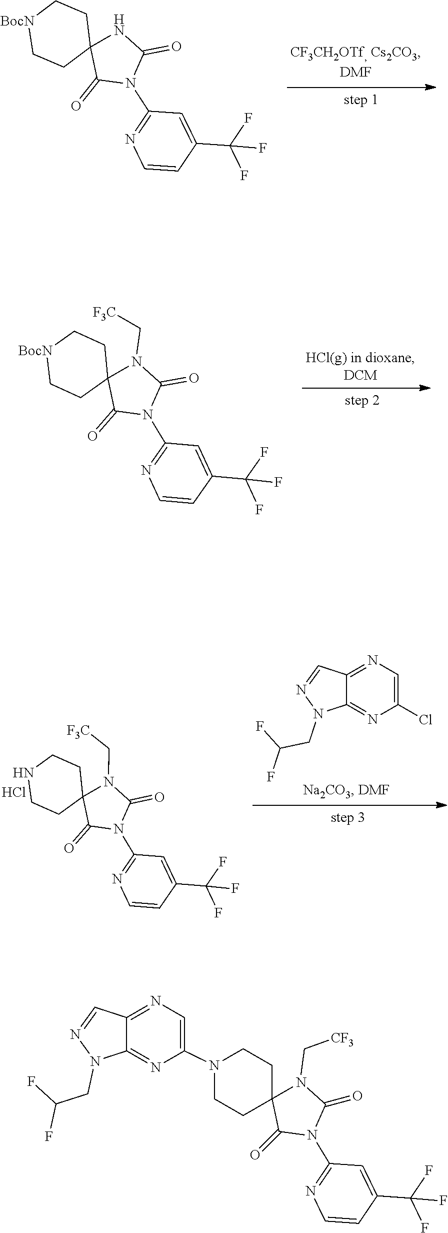 [SMILES: CC(C)(C)OC(=O)N1CCC2(CC1)NC(=O)N(c1cc(C(F)(F)F)ccn1)C2=O ; CC(C)(C)OC(=O)N1CCC2(CC1)C(=O)N(c1cc(C(F)(F)F)ccn1)C(=O)N2CC(F)(F)F ; Cl ; O=C1N(c2cc(C(F)(F)F)ccn2)C(=O)C2(CCNCC2)N1CC(F)(F)F ; FC(F)Cn1ncc2ncc(Cl)nc21 ; O=C1N(c2cc(C(F)(F)F)ccn2)C(=O)C2(CCN(c3cnc4cnn(CC(F)F)c4n3)CC2)N1CC(F)(F)F]

Step 1: tert-butyl 2,4-dioxo-1-(2,2,2-trifluoroethyl)-3-(4-(trifluoromethyl)pyridin-2-yl)-1,3,8-triazaspiro[4.5]decane-8-carboxylate: A solution of tert-butyl 2,4-dioxo-3-(4-(trifluoromethyl)pyridin-2-yl)-1,3,8-triazaspiro[4.5]decane-8-carboxylate (80 mg, 0.193 mmol, 1 equiv), 2,2,2-trifluoroethyl trifluoromethanesulfonate (53.8 mg, 0.232 mmol, 1.2 equiv) and Cs₂CO₃ (126 mg, 0.386 mmol, 2 equiv) in DMF (2 mL) was stirred for 1.5 h at 50° C. The resulting mixture was extracted with EtOAc (30 mL). The combined organic layers were washed with water (3×10 mL), dried over anhydrous Na₂SO₄. After filtration, the filtrate was concentrated under reduced pressure. The residue was purified by silica gel column chromatography, eluted with PE:EtOAc (1:1) to afford tert-butyl 2,4-dioxo-1-(2,2,2-trifluoroethyl)-3-(4-(trifluoromethyl)pyridin-2-yl)-1,3,8-triazaspiro[4.5]decane-8-carboxylate (40 mg, 41.74%) as a white solid. MS m/z: 497 [M+H]⁺.

Step 2: 1-(2,2,2-trifluoroethyl)-3-(4-(trifluoromethyl)pyridin-2-yl)-1,3,8-triazaspiro[4.5]decane-2,4-dione hydrochloride: Followed the general procedure B using tert-butyl 2,4-dioxo-1-(2,2,2-trifluoroethyl)-3-(4-(trifluoromethyl)pyridin-2-yl)-1,3,8-triazaspiro[4.5]decane-8-carboxylate (40 mg, 0.081 mmol, 1 equiv) as the starting material to give the crude product 1-(2,2,2-trifluoroethyl)-3-(4-(trifluoromethyl)pyridin-2-yl)-1,3,8-triazaspiro[4.5]decane-2,4-dione hydrochloride (27.2 mg) was used in the next step directly without further purification. MS m/z: 397 [M+H]⁺.

Step 3: 8-(1-(2,2-difluoroethyl)-1H-pyrazolo[3,4-b]pyrazin-6-yl)-1-(2,2,2-trifluoroethyl)-3-(4-(trifluoromethyl)pyridin-2-yl)-1,3,8-triazaspiro[4.5]decane-2,4-dione: A solution of 1-(2,2,2-trifluoroethyl)-3-(4-(trifluoromethyl)pyridin-2-yl)-1,3,8-triazaspiro[4.5]decane-2,4-dione hydrochloride (27.2 mg, 0.063 mmol, 1 equiv), 6-chloro-1-(2,2-difluoroethyl)pyrazolo[3,4-b]pyrazine (15.17 mg, 0.069 mmol, 1.1 equiv) and Na₂CO₃ (13.37 mg, 0.126 mmol, 2 equiv) in DMF (1 mL) was stirred for 2 h at 100° C. The residue was purified by reverse flash chromatography with the following conditions: column, C18 silica gel; mobile phase, MeCN in water, 10% to 100% gradient in 30 min; detector, UV 254 nm. This resulted in 8-(1-(2,2-difluoroethyl)-1H-pyrazolo[3,4-b]pyrazin-6-yl)-1-(2,2,2-trifluoroethyl)-3-(4-(trifluoromethyl)pyridin-2-yl)-1,3,8-triazaspiro[4.5]decane-2,4-dione (9 mg, 24.42%) as a white solid. ¹H NMR (400 MHz, Methanol-d₄) δ 8.86 (d, J=5.2 Hz, 1H), 8.43 (s, 1H), 8.03 (s, 1H), 7.96 (s, 1H), 7.83 (d, J=5.1 Hz, 1H), 6.27 (tt, J=55.3, 4.1 Hz, 1H), 4.76-4.64 (m, 4H), 4.19 (t, J=9.0 Hz, 2H), 3.85-3.77 (m, 2H), 2.31-2.17 (m, 4H). MS m/z: 579.1 [M+H]+

### 8-(1-(2,2-difluoroethyl)-1H-pyrazolo[3,4-b]pyrazin-6-yl)-1-ethyl-3-(4-(trifluoromethyl)pyridin-2-yl)-1,3,8-triazaspiro[4.5]decane-2,4-dione (72)

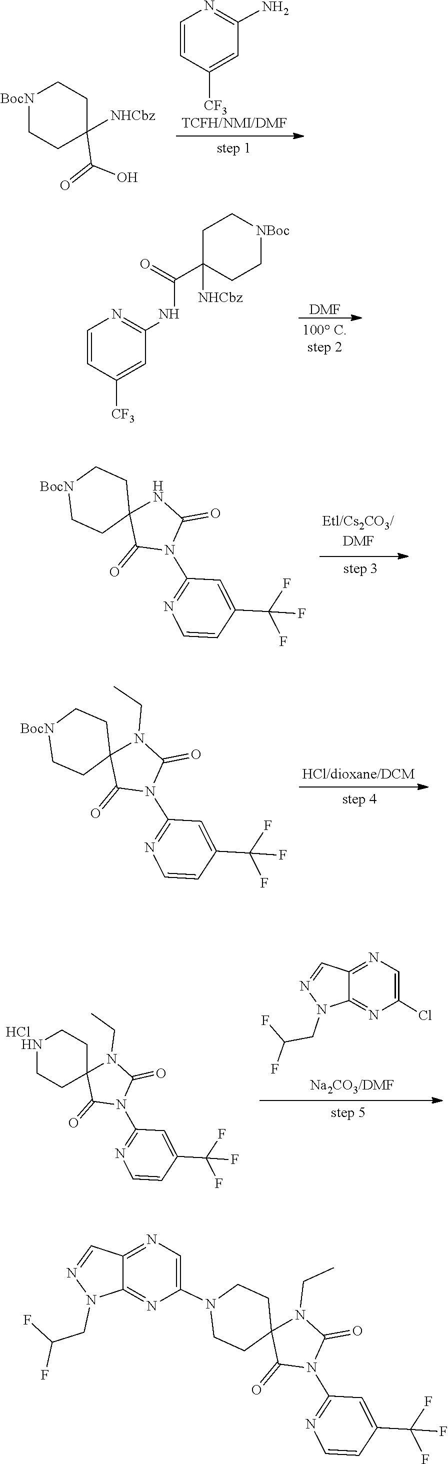

Step 1: tert-butyl 4-(((benzyloxy)carbonyl)amino)-4-((4-(trifluoromethyl)pyridin-2-yl)carbamoyl)piperidine-1-carboxylate: A solution of 4-(((benzyloxy)carbonyl)amino)-1-(tert-butoxycarbonyl)piperidine-4-carboxylic acid (1 g, 2.643 mmol, 1 equiv), 4-(trifluoromethyl)pyridin-2-amine (0.51 g, 3.17 mmol, 1.2 equiv), TCFH (1.11 g, 3.964 mmol, 1.5 equiv) and NMI (0.65 g, 7.93 mmol, 3 equiv) in ACN (10 mL) was stirred for overnight at 60° C. The resulting mixture was concentrated under reduced pressure. The residue was purified by silica gel column chromatography, eluted with PE/EtOAc (1:1) to afford tert-butyl 4-(((benzyloxy)carbonyl)amino)-4-((4-(trifluoromethyl)pyridin-2-yl)carbamoyl)piperidine-1-carboxylate (450 mg, 32.6%) as a colorless oil. MS m/z: 523 [M+H]⁺.

Step 2: tert-butyl 2,4-dioxo-3-(4-(trifluoromethyl)pyridin-2-yl)-1,3,8-triazaspiro[4.5]decane-8-carboxylate: A solution of tert-butyl 4-(((benzyloxy)carbonyl)amino)-4-((4-(trifluoromethyl)pyridin-2-yl)carbamoyl)piperidine-1-carboxylate (450 mg, 0.861 mmol, 1 equiv) in DMF (5 mL) was stirred for overnight at 120° C. The residue was purified by reverse flash chromatography with the following conditions: column, C18 silica gel; mobile phase, MeCN in water, 10% to 100% gradient in 30 min; detector, UV 254 nm. This resulted in tert-butyl 2,4-dioxo-3-(4-(trifluoromethyl)pyridin-2-yl)-1,3,8-triazaspiro[4.5]decane-8-carboxylate (250 mg, 70.0%) as a white solid. MS m/z: 415 [M+H]⁺.

Step 3: tert-butyl 1-ethyl-2,4-dioxo-3-(4-(trifluoromethyl)pyridin-2-yl)-1,3,8-triazaspiro[4.5]decane-8-carboxylate: A solution of tert-butyl 2,4-dioxo-3-(4-(trifluoromethyl)pyridin-2-yl)-1,3,8-triazaspiro[4.5]decane-8-carboxylate (80 mg, 0.193 mmol, 1 equiv), iodoethane (36.13 mg, 0.232 mmol, 1.2 equiv) and Cs₂CO₃ (125.80 mg, 0.386 mmol, 2 equiv) in DMF (2 mL) was stirred for 1.5 h at 50° C. The resulting mixture was extracted with EtOAc (30 mL). The combined organic layers were washed with water (3×10 mL), dried over anhydrous Na₂SO₄. After filtration, the filtrate was concentrated under reduced pressure. The residue was purified by silica gel column chromatography, eluted with PE/EtOAc (1:1) to afford tert-butyl 1-ethyl-2,4-dioxo-3-(4-(trifluoromethyl)pyridin-2-yl)-1,3,8-triazaspiro[4.5]decane-8-carboxylate (60 mg, 70.24%) as a white solid. MS m/z: 443 [M+H]⁺.

Step 4: 1-ethyl-3-(4-(trifluoromethyl)pyridin-2-yl)-1,3,8-triazaspiro[4.5]decane-2,4-dione hydrochloride: Followed the general procedure B using tert-butyl 1-ethyl-2,4-dioxo-3-[4-(trifluoromethyl)pyridin-2-yl]-1,3,8-triazaspiro[4.5]decane-8-carboxylate (60 mg, 0.136 mmol, 1 equiv) as the starting material to give crude product 1-ethyl-3-(4-(trifluoromethyl)pyridin-2-yl)-1,3,8-triazaspiro[4.5]decane-2,4-dione hydrochloride (30 mg) was used in the next step directly without further purification. MS m/z: 343 [M+H]⁺.

Step 5: 8-(1-(2,2-difluoroethyl)-1H-pyrazolo[3,4-b]pyrazin-6-yl)-1-ethyl-3-(4-(trifluoromethyl)pyridin-2-yl)-1,3,8-triazaspiro[4.5]decane-2,4-dione: A solution of 1-ethyl-3-(4-(trifluoromethyl)pyridin-2-yl)-1,3,8-triazaspiro[4.5]decane-2,4-dione (27.6 mg, 0.073 mmol, 1 equiv), 6-chloro-1-(2,2-difluoroethyl)pyrazolo[3,4-b]pyrazine (17.56 mg, 0.080 mmol, 1.1 equiv) and Na₂CO₃ (15.48 mg, 0.146 mmol, 2 equiv) in DMF (1 mL) was stirred for 2 h at 100° C. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reverse flash chromatography with the following conditions: column, C18 silica gel; mobile phase, MeCN in water, 10% to 100% gradient in 30 min; detector, UV 254 nm. This resulted in 8-(1-(2,2-difluoroethyl)-1H-pyrazolo[3,4-b]pyrazin-6-yl)-1-ethyl-3-(4-(trifluoromethyl)pyridin-2-yl)-1,3,8-triazaspiro[4.5]decane-2,4-dione (10.7 mg, 27.82%) as a white solid. ¹H NMR (400 MHz, Methanol-d₄) δ 8.85 (d, J=5.2 Hz, 1H), 8.43 (d, J=2.5 Hz, 1H), 8.03 (d, J=1.3 Hz, 1H), 7.96-7.90 (m, 1H), 7.87-7.72 (m, 1H), 6.28 (tt, J=55.3, 4.1 Hz, 1H), 4.78-4.65 (m, 4H), 3.83-3.76 (m, 2H), 3.39 (q, J=7.1 Hz, 2H), 2.27-2.08 (m, 4H), 1.26 (t, J=7.1 Hz, 3H). MS m/z: 525.1 [M+H]⁺.

### 8-(1-(2,2-difluoroethyl)-1H-pyrazolo[3,4-b]pyrazin-6-yl)-4,4-dimethyl-2-(4-(trifluoromethyl)pyridin-2-yl)-2,8-diazaspiro[4.5]decan-3-one (73)

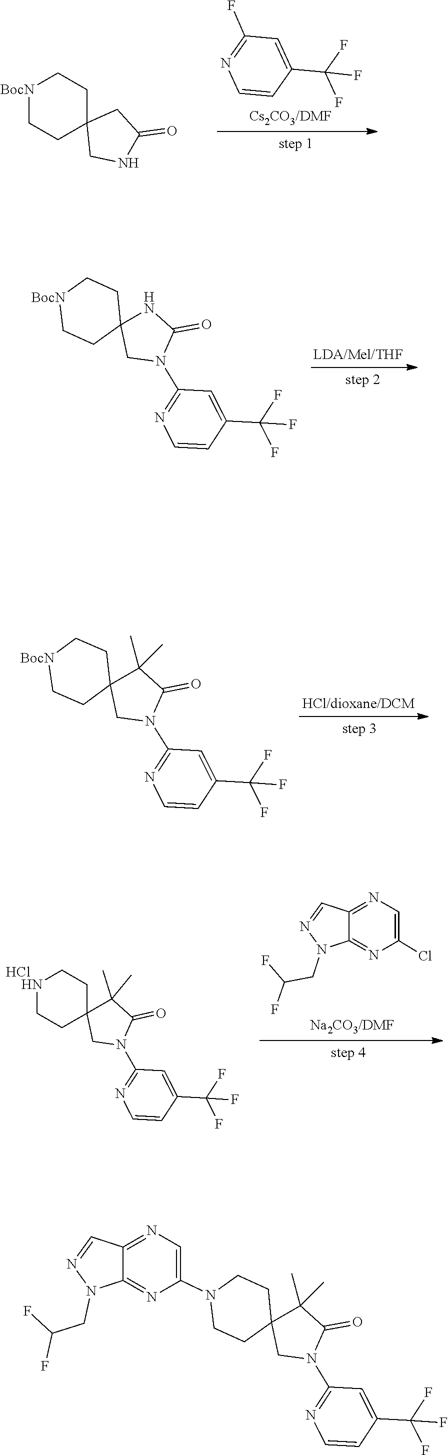

Step 1: tert-butyl 3-oxo-2-(4-(trifluoromethyl)pyridin-2-yl)-2,8-diazaspiro[4.5]decane-8-carboxylate: To a stirred solution of tert-butyl 3-oxo-2,8-diazaspiro[4.5]decane-8-carboxylate (500 mg, 1.966 mmol, 1 equiv) and 2-fluoro-4-(trifluoromethyl)pyridine (389.47 mg, 2.359 mmol, 1.2 equiv) in DMF (10 mL) was added Cs₂CO₃ (1281.09 mg, 3.932 mmol, 2 equiv). The resulting mixture was stirred for 16 h at 100° C. under nitrogen atmosphere. The reaction was diluted with water (20 mL) and extracted with EtOAc (25 mL×2). The combined organic phases were washed with water (40 mL), brine (40 mL), dried over anhydrous Na₂SO₄, filtered and concentrated to dryness under vacuum. The residue was purified by chromatography on silica gel (EtOAc:PE=1:1) to afford tert-butyl 3-oxo-2-(4-(trifluoromethyl)pyridin-2-yl)-2,8-diazaspiro[4.5]decane-8-carboxylate (400 mg, 50.94%) as a white solid. MS m/z: [M−tBu+H]⁺.

Step 2: tert-butyl 4,4-dimethyl-3-oxo-2-(4-(trifluoromethyl)pyridin-2-yl)-2,8-diazaspiro[4.5]decane-8-carboxylate: To a stirred solution of tert-butyl 3-oxo-2-(4-(trifluoromethyl)pyridin-2-yl)-2,8-diazaspiro[4.5]decane-8-carboxylate (200 mg, 0.500 mmol, 1 equiv) in THF (3 mL) was added a solution of lithium diisopropylamide (1 M in THF, 1.1 mmol, 1.1 mL, 2.2 equiv) under nitrogen atmosphere at −40° C. dropwise. The mixture was warmed up to ambient temperature and stirred for 1 h. The mixture was then cooled to 0° C. and iodomethane (212 mg, 1.50 mmol, 3 equiv) was added. The mixture was warmed to ambient temperature and stirred overnight. The mixture was quenched by sat. NH₄Cl (aq.) (5 mL), extracted with EtOAc (10 mL×2). The combined organic phase was washed with water (20 mL), brine (20 mL), dried over anhydrous Na₂SO₄, filtered and concentrated to dryness under vacuum. The residue was purified by chromatography on silica gel (EtOAc:PE=1:1) to afforded tert-butyl 4,4-dimethyl-3-oxo-2-(4-(trifluoromethyl)pyridin-2-yl)-2,8-diazaspiro[4.5]decane-8-carboxylate (60 mg, 28.1%). MS m/z: 428 [M−tBu+H]⁺.

Step 3: 4,4-dimethyl-2-[4-(trifluoromethyl)pyridin-2-yl]-2,8-diazaspiro[4.5]decan-3-one hydrochloride: Followed the general procedure B using tert-butyl 4,4-dimethyl-3-oxo-2-[4-(trifluoromethyl)pyridin-2-yl]-2,8-diazaspiro[4.5]decane-8-carboxylate (60 mg, 0.140 mmol, 1 equiv) as the starting material to afford crude product 4,4-dimethyl-2-[4-(trifluoromethyl)pyridin-2-yl]-2,8-diazaspiro[4.5]decan-3-one hydrochloride (40 mg) as a white solid. MS m/z: 328 [M+H]⁺.

Step 4: 8-(1-(2,2-difluoroethyl)-1H-pyrazolo[3,4-b]pyrazin-6-yl)-4,4-dimethyl-2-(4-(trifluoromethyl)pyridin-2-yl)-2,8-diazaspiro[4.5]decan-3-one: To a stirred solution of 4,4-dimethyl-2-[4-(trifluoromethyl)pyridin-2-yl]-2,8-diazaspiro[4.5]decan-3-one hydrochloride (40 mg, 0.110 mmol, 1 equiv) and 6-chloro-1-(2,2-difluoroethyl)pyrazolo[3,4-b]pyrazine (28.84 mg, 0.132 mmol, 1.2 equiv) in DMF (1 mL) was added Na₂CO₃ (34.96 mg, 0.330 mmol, 3 equiv). The resulting mixture was stirred for 2 h at 100° C. under nitrogen atmosphere. The resulting mixture was diluted with water (20 mL) and extracted with EtOAc (15 mL×3). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 8-(1-(2,2-difluoroethyl)-1H-pyrazolo[3,4-b]pyrazin-6-yl)-4,4-dimethyl-2-(4-(trifluoromethyl)pyridin-2-yl)-2,8-diazaspiro[4.5]decan-3-one (10.7 mg, 18.93%) as a white solid. ¹H NMR (400 MHz, DMSO-d6) δ 8.73-8.64 (m, 2H), 8.48 (s, 1H), 8.13 (s, 1H), 7.56-7.52 (m, 1H), 6.60-6.26 (m, 1H), 4.76-4.64 (m, 2H), 4.55 (d, J=13.5 Hz, 2H), 4.14 (s, 2H), 3.20-3.08 (m, 2H), 1.69-1.59 (m, 4H), 1.05 (s, 6H). MS m/z: 510.2 [M+H]⁺.

### 8-(1-(2,2-difluoroethyl)-1H-pyrazolo[3,4-b]pyrazin-6-yl)-1-isopropyl-3-(4-(trifluoromethyl)pyridin-2-yl)-1,3,8-triazaspiro[4.5]decane-2,4-dione (74)

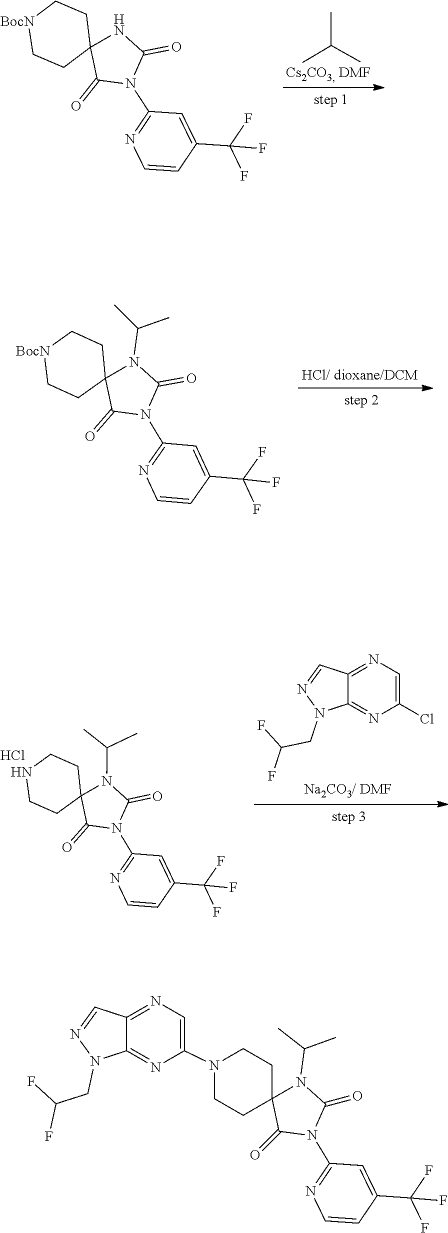

Step 1: tert-butyl 1-isopropyl-2,4-dioxo-3-(4-(trifluoromethyl)pyridin-2-yl)-1,3,8-triazaspiro[4.5]decane-8-carboxylate: A solution of tert-butyl 2,4-dioxo-3-(4-(trifluoromethyl)pyridin-2-yl)-1,3,8-triazaspiro[4.5]decane-8-carboxylate (80 mg, 0.193 mmol, 1 equiv), 2-iodopropane (39.38 mg, 0.232 mmol, 1.2 equiv) and Cs₂CO₃ (125.80 mg, 0.386 mmol, 2 equiv) in DMF (2 mL) was stirred for 1.5 h at 50° C. The resulting mixture was diluted with EtOAc (30 mL). The combined organic layers were washed with water (3×10 mL), dried over anhydrous Na₂SO₄. After filtration, the filtrate was concentrated under reduced pressure. The residue was purified by silica gel column chromatography, eluted with PE/EtOAc (1:1) to afford tert-butyl 1-isopropyl-2,4-dioxo-3-(4-(trifluoromethyl)pyridin-2-yl)-1,3,8-triazaspiro[4.5]decane-8-carboxylate as a white solid (35 mg, 39.72%). MS m/z: 457 [M+H]⁺.

Step 2: 1-isopropyl-3-(4-(trifluoromethyl)pyridin-2-yl)-1,3,8-triazaspiro[4.5]decane-2,4-dione hydrochloride: Followed the general procedure B using tert-butyl 1-isopropyl-2,4-dioxo-3-(4-(trifluoromethyl)pyridin-2-yl)-1,3,8-triazaspiro[4.5]decane-8-carboxylate (35 mg, 0.077 mmol, 1 equiv) as starting material to afford crude product 1-isopropyl-3-(4-(trifluoromethyl)pyridin-2-yl)-1,3,8-triazaspiro[4.5]decane-2,4-dione hydrochloride (22.0 mg) was used in the next step directly without further purification. MS m/z: 357[M+H]⁺.

Step 3: 8-(1-(2,2-difluoroethyl)-1H-pyrazolo[3,4-b]pyrazin-6-yl)-1-isopropyl-3-(4-(trifluoromethyl)pyridin-2-yl)-1,3,8-triazaspiro[4.5]decane-2,4-dione: A solution of 1-isopropyl-3-(4-(trifluoromethyl)pyridin-2-yl)-1,3,8-triazaspiro[4.5]decane-2,4-dione hydrochloride (22.0 mg, 0.056 mmol, 1 equiv) and Na₂CO₃ (11.90 mg, 0.112 mmol, 2 equiv) in DMF (1 mL) was stirred for 2 h at 100° C. The resulting mixture was diluted with water (20 mL) and extracted with EtOAc (15 mL×3). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). This resulted in 8-(1-(2,2-difluoroethyl)-1H-pyrazolo[3,4-b]pyrazin-6-yl)-1-isopropyl-3-(4-(trifluoromethyl)pyridin-2-yl)-1,3,8-triazaspiro[4.5]decane-2,4-dione (5 mg, 15.72%) as a white solid. ¹H NMR (400 MHz, Methanol-d₄) δ 8.85 (d, J=5.2 Hz, 1H), 8.43 (s, 1H), 8.03 (s, 1H), 7.92 (s, 1H), 7.80 (d, J=5.3 Hz, 1H), 6.28 (tt, J=55.4, 4.0 Hz, 1H), 4.73-4.58 (m, 4H), 3.87-3.75 (m, 2H), 3.58-3.50 (m, 1H), 2.25-2.10 (m, 4H), 1.45 (d, J=6.8 Hz, 6H). MS m/z: 539.2 [M+H]+

### 8-(1-(2,2-difluoroethyl)-1H-pyrazolo[3,4-b]pyrazin-6-yl)-4-ethyl-2-(4-(trifluoromethyl)pyridin-2-yl)-2,8-diazaspiro[4.5]decan-3-one (75)

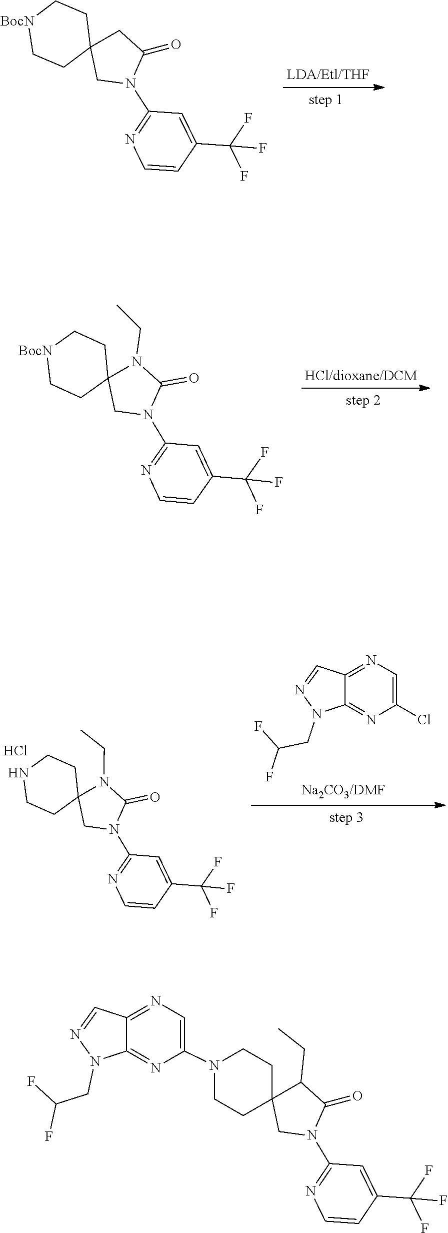 [SMILES: CC(C)(C)OC(=O)N1CCC2(CC1)CC(=O)N(c1cc(C(F)(F)F)ccn1)C2 ; CCN1C(=O)N(c2cc(C(F)(F)F)ccn2)CC12CCN(C(=O)OC(C)(C)C)CC2 ; Cl ; CCN1C(=O)N(c2cc(C(F)(F)F)ccn2)CC12CCNCC2 ; FC(F)Cn1ncc2ncc(Cl)nc21 ; CCC1C(=O)N(c2cc(C(F)(F)F)ccn2)CC12CCN(c1cnc3cnn(CC(F)F)c3n1)CC2]

Step 1: tert-butyl 4-ethyl-3-oxo-2-(4-(trifluoromethyl)pyridin-2-yl)-2,8-diazaspiro[4.5]decane-8-carboxylate: To a stirred solution of tert-butyl 3-oxo-2-(4-(trifluoromethyl)pyridin-2-yl)-2,8-diazaspiro[4.5]decane-8-carboxylate (200 mg, 0.500 mmol, 1 equiv) in THF (3 mL) was added a solution of lithium diisopropylamide (1 M in THF, 0.55 mmol, 0.550 mL, 1.1 equiv) under nitrogen atmosphere at −40° C. dropwise. The mixture was warmed up to ambient temperature and stirred for 1 h. The mixture was then cooled to O ° C. and iodoethane (117 mg, 0.750 mmol, 1.50 equiv) was added. The mixture was warmed to ambient temperature and stirred overnight. The mixture was quenched by sat. NH₄Cl (aq.) (5 mL), extracted with EtOAc (10 mL×2). The combined organic phase was washed with water (20 mL), brine (20 mL), dried over anhydrous Na₂SO₄, filtered and concentrated to dryness under vacuum. The residue was purified by chromatography on silica gel (EtOAc:PE=1:1) to afforded tert-butyl 4-ethyl-3-oxo-2-[4-(trifluoromethyl)pyridin-2-yl]-2,8-diazaspiro[4.5]decane-8-carboxylate (100 mg, 46.8%). MS m/z: 428 [M−tBu+H]⁺.

Step 2: 4-ethyl-2-[4-(trifluoromethyl)pyridin-2-yl]-2,8-diazaspiro[4.5]decan-3-one hydrochloride: Followed the general procedure B using tert-butyl 4-ethyl-3-oxo-2-[4-(trifluoromethyl)pyridin-2-yl]-2,8-diazaspiro[4.5]decane-8-carboxylate (60 mg, 0.140 mmol, 1 equiv) as the starting material to afford the crude product 4-ethyl-2-[4-(trifluoromethyl)pyridin-2-yl]-2,8-diazaspiro[4.5]decan-3-one hydrochloride (50 mg) as a white solid. MS m/z: 328 [M+H]⁺.

Step 3: 8-(1-(2,2-difluoroethyl)-1H-pyrazolo[3,4-b]pyrazin-6-yl)-4-ethyl-2-(4-(trifluoromethyl)pyridin-2-yl)-2,8-diazaspiro[4.5]decan-3-one: To a stirred solution of 4-ethyl-2-[4-(trifluoromethyl)pyridin-2-yl]-2,8-diazaspiro[4.5]decan-3-one hydrochloride (50 mg, 0.137 mmol, 1 equiv) and 6-chloro-1-(2,2-difluoroethyl)pyrazolo[3,4-b]pyrazine (36.05 mg, 0.164 mmol, 1.2 equiv) in DMF (1 mL) was added Na₂CO₃ (43.70 mg, 0.411 mmol, 3 equiv). The resulting mixture was stirred for 2 h at 100° C. under nitrogen atmosphere. The resulting mixture was diluted with water (20 mL) and extracted with EtOAc (15 mL×3). The combined organic layers were washed with brine (30 mL), dried over anhydrous Na₂SO₄. After filtration, the filtrate was concentrated to dryness under reduced pressure. The residue was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 8-(1-(2,2-difluoroethyl)-1H-pyrazolo[3,4-b]pyrazin-6-yl)-4-ethyl-2-(4-(trifluoromethyl)pyridin-2-yl)-2,8-diazaspiro[4.5]decan-3-one (30 mg, 42.33%) as a white solid. ¹H NMR (400 MHz, DMSO-d6) δ 8.71-8.63 (m, 2H), 8.49 (s, 1H), 8.13 (s, 1H), 7.54-7.50 (m, 1H), 6.60-6.25 (m, 1H), 4.76-4.62 (m, 2H), 4.40-4.17 (m, 3H), 3.85 (d, J=11.3 Hz, 1H), 3.40 (dd, J=14.2, 3.4 Hz, 2H), 2.48-2.44 (m, 1H), 1.87-1.73 (m, 2H), 1.69-1.46 (m, 4H), 1.05 (t, J=7.4 Hz, 3H). MS m/z: 510.2 [M+H]⁺.

### 7-[1-(2,2-difluoroethyl)-1H-pyrazolo[3,4-b]pyrazin-6-yl]-2-[4-(trifluoromethyl)pyridin-3-yl]-2,7-diazaspiro[3.5]nonane (76)

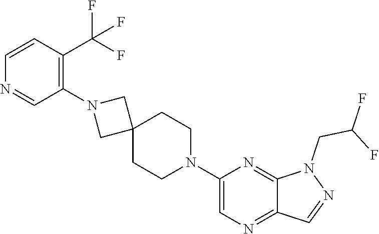 [SMILES: FC(F)Cn1ncc2ncc(N3CCC4(CC3)CN(c3cnccc3C(F)(F)F)C4)nc21]

Followed the general procedure A using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 3-fluoro-4-(trifluoromethyl)pyridine (14.4 mg, 87 μmol, 1.0 equiv) as the starting materials to give 7-[1-(2,2-difluoroethyl)-1H-pyrazolo[3,4-b]pyrazin-6-yl]-2-[4-(trifluoromethyl)pyridin-3-yl]-2,7-diazaspiro[3.5]nonane (16 mg, 41%) as a colorless oil. ¹H NMR (500 MHz, DMSO-d₆) δ 8.50 (s, 1H), 8.13 (s, 1H), 8.03 (s, 1H), 8.00 (d, J=5.1 Hz, 1H), 7.41 (d, J=5.1 Hz, 1H), 6.44 (tt, J=54.9, 3.8 Hz, 2H), 4.70 (td, J=15.0, 3.8 Hz, 2H), 3.93 (d, J=1.8 Hz, 4H), 3.86-3.73 (m, 4H), 1.94-1.83 (m, 4H). MS m/z: 454 [M+H]⁺.

### 7-[1-(2,2-difluoroethyl)-1H-pyrazolo[3,4-b]pyrazin-6-yl]-2-[6-(trifluoromethyl)pyridin-2-yl]-2,7-diazaspiro[3.5]nonane (77)

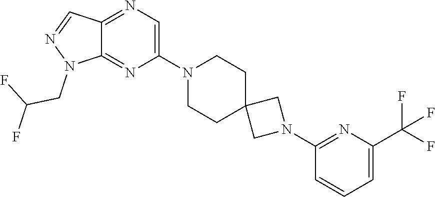 [SMILES: FC(F)Cn1ncc2ncc(N3CCC4(CC3)CN(c3cccc(C(F)(F)F)n3)C4)nc21]

Followed the general procedure A using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 2-bromo-6-(trifluoromethyl)pyridine (19.7 mg, 87 μmol, 1.0 equiv) as the starting materials to give 7-[1-(2,2-difluoroethyl)-1H-pyrazolo[3,4-b]pyrazin-6-yl]-2-[6-(trifluoromethyl)pyridin-2-yl]-2,7-diazaspiro[3.5]nonane (22 mg, 41%) as a colorless oil. ¹H NMR (500 MHz, DMSO-d₆) δ 8.50 (s, 1H), 8.13 (s, 1H), 7.72 (t, J=7.9 Hz, 1H), 7.02 (d, J=7.2 Hz, 1H), 6.65 (d, J=8.5 Hz, 1H), 6.44 (tt, J=54.8, 3.8 Hz, 1H), 4.70 (td, J=15.0, 3.8 Hz, 2H), 3.81 (s, 4H), 3.80-3.71 (m, 4H), 1.86 (t, J=5.6 Hz, 4H). MS m/z: 454 [M+H]⁺.

### 6-[1-(2,2-difluoroethyl)-1H-pyrazolo[3,4-b]pyrazin-6-yl]-2-[5-(trifluoromethyl)pyridin-2-yl]-2,6-diazaspiro[3.5]nonane (78)

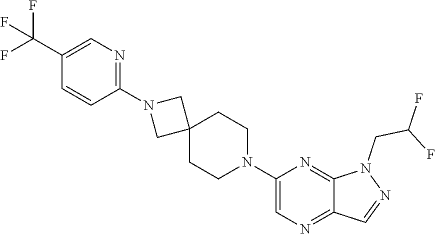 [SMILES: FC(F)Cn1ncc2ncc(N3CCC4(CC3)CN(c3ccc(C(F)(F)F)cn3)C4)nc21]

Followed the general procedure A using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 2-bromo-5-(trifluoromethyl)pyridine (19.7 mg, 87 μmol, 1.0 equiv) as the starting materials to give 7-[1-(2,2-difluoroethyl)-1H-pyrazolo[3,4-b]pyrazin-6-yl]-2-[5-(trifluoromethyl)pyridin-2-yl]-2,7-diazaspiro[3.5]nonane (24 mg, 61%) as a colorless oil. ¹H NMR (500 MHz, DMSO-d₆) δ 8.51 (s, 1H), 8.37 (dt, J=2.4, 1.0 Hz, 1H), 8.13 (s, 1H), 7.78 (dd, J=8.9, 2.5 Hz, 1H), 6.48 (d, J=8.8 Hz, 1H), 6.44 (tt, J=54.9, 3.8 Hz, 2H), 4.70 (td, J=15.1, 3.8 Hz, 2H), 3.85 (s, 4H), 3.81-3.69 (m, 4H), 1.91-1.82 (m, 4H). MS m/z: 454 [M+H]⁺.

### 7-[1-(2,2-difluoroethyl)-1H-pyrazolo[3,4-b]pyrazin-6-yl]-2-[3-(trifluoromethyl)pyridin-2-yl]-2,7-diazaspiro[3.5]nonane (79)

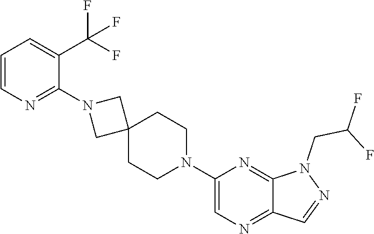 [SMILES: FC(F)Cn1ncc2ncc(N3CCC4(CC3)CN(c3ncccc3C(F)(F)F)C4)nc21]

Followed the general procedure A using 7-[1-(2,2-difluoroethyl)-1H-pyrazolo[3,4-b]pyrazin-6-yl]-2,7-diazaspiro[3.5]nonane hydrochloride (30 mg, 87 μmol, 1.0 equiv) and 2-bromo-3-(trifluoromethyl)pyridine (19.7 mg, 87 μmol, 1.0 equiv) as the starting materials to give 7-[1-(2,2-difluoroethyl)-1H-pyrazolo[3,4-b]pyrazin-6-yl]-2-[3-(trifluoromethyl)pyridin-2-yl]-2,7-diazaspiro[3.5]nonane (23 mg, 58%) as a colorless oil. ¹H NMR (500 MHz, DMSO-d₆) δ 8.50 (s, 1H), 8.35 (dd, J=4.8, 1.7 Hz, 1H), 8.13 (s, 1H), 7.87 (dd, J=7.8, 1.8 Hz, 1H), 6.78 (dd, J=7.7, 4.8 Hz, 1H), 6.44 (tt, J=54.9, 3.8 Hz, 1H), 4.70 (td, J=15.0, 3.8 Hz, 2H), 3.92 (d, J=1.6 Hz, 4H), 3.83-3.73 (m, 4H), 1.92-1.79 (m, 4H). MS m/z: 454 [M+H]⁺.

### 8-[1-(2,2-difluoroethyl)-1H-pyrazolo[3,4-b]pyrazin-6-yl]-2-{[6-(trifluoromethyl)pyridin-2-yl]oxy}-8-azaspiro[4.5]decane (80)

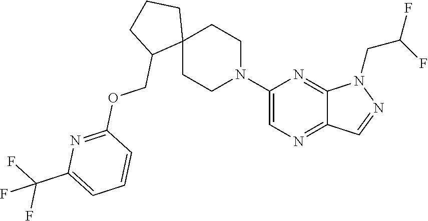 [SMILES: FC(F)Cn1ncc2ncc(N3CCC4(CCCC4COc4cccc(C(F)(F)F)n4)CC3)nc21]

Step 1: tert-butyl 1-({[6-(trifluoromethyl)pyridin-2-yl]oxy}methyl)-8-azaspiro[4.5]decane-8-carboxylate: Followed the general procedure E using tert-butyl 1-(hydroxymethyl)-8-azaspiro[4.5]decane-8-carboxylate (50 mg, 221 μmol, 1.0 equiv) and 2-bromo-6-(trifluoromethyl)pyridine (41.9 mg, 221 μmol, 1.0 equiv.) as the starting materials to tert-butyl 1-({[6-(trifluoromethyl)pyridin-2-yl]oxy}methyl)-8-azaspiro[4.5]decane-8-carboxylate (16 mg, 21%) as a colorless oil. MS m/z: 415 [M+H]⁺.

Step 2: 1-({[6-(trifluoromethyl)pyridin-2-yl]oxy}methyl)-8-azaspiro[4.5]decane hydrochloride: Followed the general procedure B using tert-butyl 1-({[6-(trifluoromethyl)pyridin-2-yl]oxy}methyl)-8-azaspiro[4.5]decane-8-carboxylate (16 mg) as the starting material to give the 1-({[6-(trifluoromethyl)pyridin-2-yl]oxy}methyl)-8-azaspiro[4.5]decane hydrochloride (16 mg). MS m/z: 315 [M+H]⁺.

Step 3: 8-[1-(2,2-difluoroethyl)-1H-pyrazolo[3,4-b]pyrazin-6-yl]-2-{[6-(trifluoromethyl)pyridin-2-yl]oxy}-8-azaspiro[4.5]decane: Followed the general procedure A using 6-chloro-1-(2,2-difluoroethyl)-1H-pyrazolo[3,4-b]pyrazine (10 mg, 46 μmol, 1.0 equiv) and 1-({[6-(trifluoromethyl)pyridin-2-yl]oxy}methyl)-8-azaspiro[4.5]decane hydrochloride (16 mg, 46 μmol, 1.0 equiv) as the starting materials to give 8-[1-(2,2-difluoroethyl)-1H-pyrazolo[3,4-b]pyrazin-6-yl]-1-({[6-(trifluoromethyl)pyridin-2-yl]oxy}methyl)-8-azaspiro[4.5]decane (15 mg, 66%) as a colorless oil. ¹H NMR (500 MHz, DMSO-d₆) δ 8.44 (s, 1H), 8.11 (s, 1H), 7.86 (t, J=7.9 Hz, 1H), 7.41 (d, J=7.3 Hz, 1H), 7.00 (d, J=8.4 Hz, 1H), 6.41 (tt, J=54.9, 3.8 Hz, 2H), 4.67 (td, J=15.0, 3.8 Hz, 2H), 4.39 (dd, J=10.7, 6.4 Hz, 1H), 4.32 (td, J=9.1, 4.6 Hz, 2H), 4.16 (dd, J=10.7, 7.4 Hz, 1H), 3.31 (s, 1H), 3.27-3.16 (m, 2H), 2.04 (p, J=7.6 Hz, 1H), 1.93-1.81 (m, 3H), 1.76-1.67 (m, 1H), 1.66-1.47 (m, 4H), 1.44 (d, J=13.3 Hz, 1H). MS m/z: 497 [M+H]⁺.

### 8-[1-(2,2-difluoroethyl)-1H-pyrazolo[3,4-b]pyrazin-6-yl]-2-{[6-(trifluoromethyl)pyridin-2-yl]oxy}-8-azaspiro[4.5]decane (81)

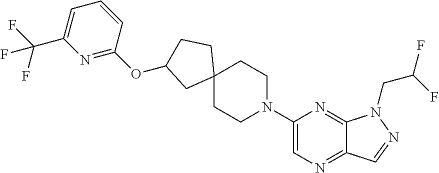 [SMILES: FC(F)Cn1ncc2ncc(N3CCC4(CCC(Oc5cccc(C(F)(F)F)n5)C4)CC3)nc21]

Step 1: tert-butyl 2-{[6-(trifluoromethyl)pyridin-2-yl]oxy}-8-azaspiro[4.5]decane-8-carboxylate: Followed the general procedure E using tert-butyl 2-hydroxy-8-azaspiro[4.5]decane-8-carboxylate (50 mg, 196 μmol, 1.0 equiv) and 2-bromo-6-(trifluoromethyl)pyridine (44.3 mg, 196 μmol, 1.0 equiv.) as the starting materials to give tert-butyl 2-{[6-(trifluoromethyl)pyridin-2-yl]oxy}-8-azaspiro[4.5]decane-8-carboxylate (15 mg, 19%) as a colorless oil. MS m/z: 401 [M+H]⁺.

Step 2: 2-{[6-(trifluoromethyl)pyridin-2-yl]oxy}-8-azaspiro[4.5]decane hydrochloride: Followed the general procedure B using tert-butyl 2-{[6-(trifluoromethyl)pyridin-2-yl]oxy}-8-azaspiro[4.5]decane-8-carboxylate (15 mg) as the starting material to give 2-{[6-(trifluoromethyl)pyridin-2-yl]oxy}-8-azaspiro[4.5]decane hydrochloride (15 mg). MS m/z: 301 [M+H]⁺.

Step 3: 8-[1-(2,2-difluoroethyl-1H-pyrazolo[3,4-b]pyrazin-6-yl]-2-{[6-(trifluoromethyl)pyridin-2-yl]oxy}-8-azaspiro[4.5]decane: Followed the general procedure A using 6-chloro-1-(2,2-difluoroethyl)-1H-pyrazolo[3,4-b]pyrazine (13.6 mg, 62.4 μmol, 1.0 equiv) and 2-{[6-(trifluoromethyl)pyridin-2-yl]oxy}-8-azaspiro[4.5]decane hydrochloride (15 mg, 62.4 μmol, 1.0 equiv) as the starting materials to give 8-[1-(2,2-difluoroethyl)-1H-pyrazolo[3,4-b]pyrazin-6-yl]-2-{[6-(trifluoromethyl)pyridin-2-yl]oxy}-8-azaspiro[4.5]decane (15 mg, 50%) as a colorless oil. ¹H NMR (500 MHz, DMSO-d₆) δ 8.46 (s, 1H), 8.11 (s, 1H), 7.95 (ddd, J=8.2, 7.3, 0.8 Hz, 1H), 7.45 (d, J=7.3 Hz, 1H), 7.11 (d, J=8.4 Hz, 1H), 6.43 (tt, J=54.9, 3.8 Hz, 2H), 5.47-5.32 (m, 1H), 4.68 (td, J=15.0, 3.8 Hz, 2H), 3.83-3.67 (m, 4H), 3.32 (m, 1H), 2.18 (dq, J=14.2, 7.4 Hz, 1H), 2.07 (dd, J=14.1, 6.8 Hz, 1H), 1.83 (dt, J=14.4, 5.6 Hz, 1H), 1.78-1.51 (m, 6H). MS m/z: 483 [M+H]⁺.

### 7-[1-(2,2-difluoroethyl)-1H-pyrazolo[3,4-b]pyrazin-6-yl]-1-({[6-(trifluoromethyl)pyridin-2-yl]oxy}methyl)-7-azaspiro[3.5]nonane (82)

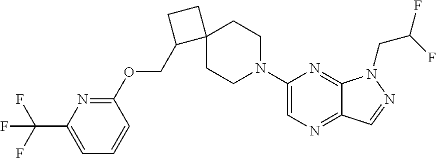 [SMILES: FC(F)Cn1ncc2ncc(N3CCC4(CCC4COc4cccc(C(F)(F)F)n4)CC3)nc21]

Step 1: tert-butyl 1-({[6-(trifluoromethyl)pyridin-2-yl]oxy}methyl)-7-azaspiro[3.5]nonane-7-carboxylate: Followed the general procedure E using tert-butyl 1-(hydroxymethyl)-7-azaspiro[3.5]nonane-7-carboxylate (50 mg, 184 μmol, 1.0 equiv) and 2-bromo-6-(trifluoromethyl)pyridine (41.6 mg, 184 μmol, 1.0 equiv.) as the starting materials to give tert-butyl 1-({[6-(trifluoromethyl)pyridin-2-yl]oxy}methyl)-7-azaspiro[3.5]nonane-7-carboxylate (42 mg, 57%) as a colorless oil. MS m/z: 401 [M+H]⁺.

Step 2: 1-({[6-(trifluoromethyl)pyridin-2-yl]oxy}methyl)-7-azaspiro[3.5]nonane hydrochloride: Followed the general procedure B using tert-butyl 1-({[6-(trifluoromethyl)pyridin-2-yl]oxy}methyl)-7-azaspiro[3.5]nonane-7-carboxylate (42 mg) as the starting material to give 1-({[6-(trifluoromethyl)pyridin-2-yl]oxy}methyl)-7-azaspiro[3.5]nonane hydrochloride (42 mg). MS m/z: 301 [M+H]⁺.

Step 3: 7-[1-(2,2-difluoroethyl)-1H-pyrazolo[3,4-b]pyrazin-6-yl]-1-({[6-(trifluoromethyl)pyridin-2-yl]oxy}methyl)-7-azaspiro[3.5]nonane: Followed the general procedure A using 6-chloro-1-(2,2-difluoroethyl)-1H-pyrazolo[3,4-b]pyrazine (13.6 mg, 62.4 μmol, 1.0 equiv) and 1-({[6-(trifluoromethyl)pyridin-2-yl]oxy}methyl)-7-azaspiro[3.5]nonane hydrochloride (21 mg, 62.4 μmol, 1.0 equiv) as the starting materials to give 7-[1-(2,2-difluoroethyl)-1H-pyrazolo[3,4-b]pyrazin-6-yl]-1-({[6-(trifluoromethyl)pyridin-2-yl]oxy}methyl)-7-azaspiro[3.5]nonane (20 mg, 66%) as a colorless oil. ¹H NMR (500 MHz, DMSO-d₆) δ 8.45 (s, 1H), 8.11 (s, 1H), 7.94-7.84 (m, 1H), 7.44 (d, J=7.2 Hz, 1H), 7.06 (d, J=8.4 Hz, 1H), 6.42 (tt, J=54.9, 3.8 Hz, 1H), 4.67 (td, J=15.0, 3.8 Hz, 2H), 4.39 (dd, J=10.9, 8.6 Hz, 1H), 4.28 (dd, J=10.9, 6.2 Hz, 1H), 4.26-4.14 (m, 2H), 3.27-3.12 (m, 2H), 2.48-2.41 (m, 1H), 2.10-1.99 (m, 1H), 1.97-1.86 (m, 1H), 1.85-1.75 (m, 3H), 1.70 (dd, J=7.4, 4.2 Hz, 2H), 1.63 (td, J=12.8, 12.1, 4.0 Hz, 1H). MS m/z: 483 [M+H]⁺.

### 7-[1-(2,2-difluoroethyl)-1H-pyrazolo[3,4-b]pyrazin-6-yl]-1-{[6-(trifluoromethyl)pyridin-2-yl]oxy}-7-azaspiro[3.5]nonane (83)

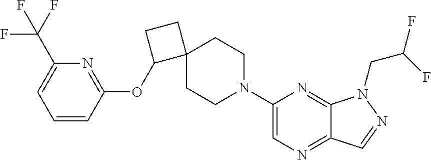 [SMILES: FC(F)Cn1ncc2ncc(N3CCC4(CCC4Oc4cccc(C(F)(F)F)n4)CC3)nc21]

Step 1: tert-butyl 1-{[6-(trifluoromethyl)pyridin-2-yl]oxy}-7-azaspiro[3.5]nonane-7-carboxylate: Followed the general procedure E using tert-butyl 1-hydroxy-7-azaspiro[3.5]nonane-7-carboxylate (50 mg, 207 μmol, 1.0 equiv) and 2-bromo-6-(trifluoromethyl)pyridine (46.8 mg, 207 μmol, 1.0 equiv.) as the starting materials to give tert-butyl 1-{[6-(trifluoromethyl)pyridin-2-yl]oxy}-7-azaspiro[3.5]nonane-7-carboxylate (27 mg, 34%) as a colorless oil. MS m/z: 387 [M+H]⁺.

Step 2: 1-{[6-(trifluoromethyl)pyridin-2-yl]oxy}-7-azaspiro[3.5]nonane hydrochloride: Followed the general procedure B using tert-butyl 1-{[6-(trifluoromethyl)pyridin-2-yl]oxy}-7-azaspiro[3.5]nonane-7-carboxylate (27 mg) as the starting material to give 1-({[6-(trifluoromethyl)pyridin-2-yl]oxy}methyl)-7-azaspiro[3.5]nonane hydrochloride (27 mg). MS m/z: 287 [M+H]⁺.

Step 3: 7-[1-(2,2-difluoroethyl)-1H-pyrazolo[3,4-b]pyrazin-6-yl]-1-{[6-(trifluoromethyl)pyridin-2-yl]oxy}-7-azaspiro[3.5]nonane: Followed the general procedure A using 6-chloro-1-(2,2-difluoroethyl)-1H-pyrazolo[3,4-b]pyrazine (18.3 mg, 83.6 μmol, 1.0 equiv) and 1-{[6-(trifluoromethyl)pyridin-2-yl]oxy}-7-azaspiro[3.5]nonane hydrochloride (27 mg, 93.6 μmol, 1.0 equiv) as the starting materials to give 7-[1-(2,2-difluoroethyl)-1H-pyrazolo[3,4-b]pyrazin-6-yl]-1-{[6-(trifluoromethyl)pyridin-2-yl]oxy}-7-azaspiro[3.5]nonane (29 mg, 74%) as a colorless oil. ¹H NMR (500 MHz, DMSO-d₆) δ 8.43 (s, 1H), 8.10 (s, 1H), 7.92 (t, J=7.9 Hz, 1H), 7.41 (d, J=7.2 Hz, 1H), 7.11 (d, J=8.4 Hz, 1H), 6.40 (tt, J=54.9, 3.8 Hz, 1H), 5.01 (t, J=7.7 Hz, 1H), 4.66 (td, J=15.0, 3.8 Hz, 2H), 4.33-4.16 (m, 2H), 3.31-3.23 (m, 1H), 3.18-3.08 (m, 1H), 2.45-2.33 (m, 1H), 2.22-2.09 (m, 1H), 2.02-1.87 (m, 2H), 1.78 (dd, J=37.4, 13.4 Hz, 2H), 1.64-1.50 (m, 2H). MS m/z: 469 [M+H]⁺.

### 7-[1-(2,2-difluoroethyl)-1H-pyrazolo[3,4-b]pyrazin-6-yl]-2-{[6-(trifluoromethyl)pyridin-2-yl]oxy}-7-azaspiro[3.5]nonane (84)

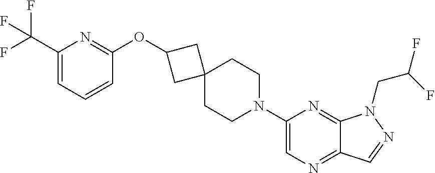 [SMILES: FC(F)Cn1ncc2ncc(N3CCC4(CC3)CC(Oc3cccc(C(F)(F)F)n3)C4)nc21]

Step 1: tert-butyl 2-{[6-(trifluoromethyl)pyridin-2-yl]oxy}-7-azaspiro[3.5]nonane-7-carboxylate: Followed the general procedure E using tert-butyl 2-hydroxy-7-azaspiro[3.5]nonane-7-carboxylate (50 mg, 207 μmol, 1.0 equiv) and 2-bromo-6-(trifluoromethyl)pyridine (46.8 mg, 207 μmol, 1.0 equiv.) as the starting materials to give tert-butyl 2-{[6-(trifluoromethyl)pyridin-2-yl]oxy}-7-azaspiro[3.5]nonane-7-carboxylate (51 mg, 64%) as a colorless oil. MS m/z: 387 [M+H]⁺.

Step 2: 2-{[6-(trifluoromethyl)pyridin-2-yl]oxy}-7-azaspiro[3.5]nonane hydrochloride: Followed the general procedure B using tert-butyl 2-{[6-(trifluoromethyl)pyridin-2-yl]oxy}-7-azaspiro[3.5]nonane-7-carboxylate (51 mg) as the starting material to give 2-{[6-(trifluoromethyl)pyridin-2-yl]oxy}-7-azaspiro[3.5]nonane hydrochloride (51 mg). MS m/z: 287 [M+H]⁺.

Step 3: 7-[1-(2,2-difluoroethyl)-1H-pyrazolo[3,4-b]pyrazin-6-yl]-2-{[6-(trifluoromethyl)pyridin-2-yl]oxy}-7-azaspiro[3.5]nonane: Followed the general procedure A using 6-chloro-1-(2,2-difluoroethyl)-1H-pyrazolo[3,4-b]pyrazine (17.6 mg, 80.5 μmol, 1.0 equiv) and 2-{[6-(trifluoromethyl)pyridin-2-yl]oxy}-7-azaspiro[3.5]nonane hydrochloride (26 mg, 80.5 μmol, 1.0 equiv) as the starting materials to give 7-[1-(2,2-difluoroethyl)-1H-pyrazolo[3,4-b]pyrazin-6-yl]-2-{[6-(trifluoromethyl)pyridin-2-yl]oxy}-7-azaspiro[3.5]nonane (28 mg, 74%) as a colorless oil. ¹H NMR (500 MHz, DMSO-d₆) δ 8.47 (s, 1H), 8.12 (s, 1H), 8.01-7.91 (m, 1H), 7.47 (d, J=7.3 Hz, 1H), 7.14 (dd, J=8.4, 0.9 Hz, 1H), 6.43 (tt, J=54.9, 3.8 Hz, 1H), 5.20 (p, J=7.0 Hz, 1H), 4.68 (td, J=15.0, 3.8 Hz, 2H), 3.79-3.71 (m, 2H), 3.71-3.59 (m, 2H), 2.47 (dd, J=7.4, 2.8 Hz, 2H), 1.97-1.87 (m, 2H), 1.75-1.69 (m, 2H), 1.69-1.61 (m, 2H). MS m/z: 469 [M+H]⁺.

### 7-[1-(2,2-difluoroethyl)-1H-pyrazolo[3,4-b]pyrazin-6-yl]-2-({[6-(trifluoromethyl)pyridin-2-yl]oxy}methyl)-7-azaspiro[3.5]nonane (85)

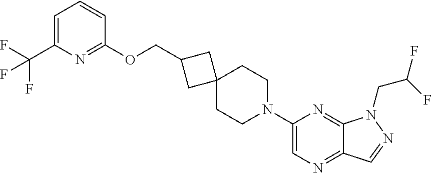 [SMILES: FC(F)Cn1ncc2ncc(N3CCC4(CC3)CC(COc3cccc(C(F)(F)F)n3)C4)nc21]

Step 1: tert-butyl 2-({[6-(trifluoromethyl)pyridin-2-yl]oxy}methyl)-7-azaspiro[3.5]nonane-7-carboxylate: Followed the general procedure E using tert-butyl 2-(hydroxymethyl)-7-azaspiro[3.5]nonane-7-carboxylate (50 mg, 196 μmol, 1.0 equiv) and 2-bromo-6-(trifluoromethyl)pyridine (44.3 mg, 196 μmol, 1.0 equiv.) as the starting materials to give tert-butyl 2-({[6-(trifluoromethyl)pyridin-2-yl]oxy}methyl)-7-azaspiro[3.5]nonane-7-carboxylate (57 mg, 73%) as a colorless oil. MS m/z: 401 [M+H]⁺.

Step 2: 2-({[6-(trifluoromethyl)pyridin-2-yl]oxy}methyl)-7-azaspiro[3.5]nonane hydrochloride: Followed the general procedure B using tert-butyl 2-({[6-(trifluoromethyl)pyridin-2-yl]oxy}methyl)-7-azaspiro[3.5]nonane-7-carboxylate (57 mg) as the starting material to give 2-({[6-(trifluoromethyl)pyridin-2-yl]oxy}methyl)-7-azaspiro[3.5]nonane hydrochloride (57 mg). MS m/z: 301 [M+H]⁺.

Step 3: 7-[1-(2,2-difluoroethyl)-1H-pyrazolo[3,4-b]pyrazin-6-yl]-2-({[6-(trifluoromethyl)pyridin-2-yl]oxy}methyl)-7-azaspiro[3.5]nonane: Followed the general procedure A using 6-chloro-1-(2,2-difluoroethyl)-1H-pyrazolo[3,4-b]pyrazine (18.8 mg, 86.1 μmol, 1.0 equiv) and 2-({[6-(trifluoromethyl)pyridin-2-yl]oxy}methyl)-7-azaspiro[3.5]nonane hydrochloride (29 mg, 86.1 μmol, 1.0 equiv) as the starting materials to give 7-[1-(2,2-difluoroethyl)-1H-pyrazolo[3,4-b]pyrazin-6-yl]-2-({[6-(trifluoromethyl)pyridin-2-yl]oxy}methyl)-7-azaspiro[3.5]nonane (29 mg, 70%) as a colorless oil. ¹H NMR (500 MHz, DMSO-d₆) δ 8.46 (s, 1H), 8.11 (s, 1H), 7.96 (t, J=7.9 Hz, 1H), 7.47 (d, J=7.3 Hz, 1H), 7.14 (d, J=8.4 Hz, 1H), 6.43 (tt, J=54.9, 3.8 Hz, 1H), 4.68 (td, J=15.0, 3.8 Hz, 2H), 4.31 (d, J=6.7 Hz, 2H), 3.75-3.69 (m, 2H), 3.68-3.59 (m, 2H), 2.75 (ddd, J=15.6, 8.8, 7.1 Hz, 1H), 2.04-1.95 (m, 2H), 1.73-1.65 (m, 4H), 1.63-1.57 (m, 2H). MS m/z: 469 [M+H]⁺.

### 8-[1-(2,2-difluoroethyl)-1H-pyrazolo[3,4-b]pyrazin-6-yl]-2-{[2-(trifluoromethyl)pyridin-3-yl]oxy}-8-azaspiro[4.5]decane (86)

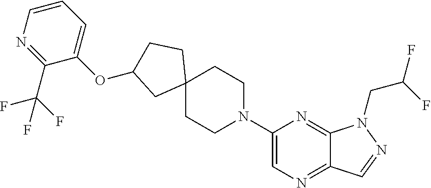 [SMILES: FC(F)Cn1ncc2ncc(N3CCC4(CCC(Oc5cccnc5C(F)(F)F)C4)CC3)nc21]

Step 1: tert-butyl 2-{[2-(trifluoromethyl)pyridin-3-yl]oxy}-8-azaspiro[4.5]decane-8-carboxylate: Followed the general procedure E using tert-butyl 2-hydroxy-8-azaspiro[4.5]decane-8-carboxylate (50 mg, 196 μmol, 1.0 equiv) and 3-fluoro-2-(trifluoromethyl)pyridine (32.3 mg, 196 μmol, 1.0 equiv.) as the starting materials to give tert-butyl 2-{[2-(trifluoromethyl)pyridin-3-yl]oxy}-8-azaspiro[4.5]decane-8-carboxylate (51 mg, 65%) as a colorless oil. MS m/z: 401 [M+H]⁺.

Step 2: 2-{[2-(trifluoromethyl)pyridin-3-yl]oxy}-8-azaspiro[4.5]decane hydrochloride: Followed the general procedure B using tert-butyl 2-{[2-(trifluoromethyl)pyridin-3-yl]oxy}-8-azaspiro[4.5]decane-8-carboxylate (51 mg) as the starting material to give 2-{[2-(trifluoromethyl)pyridin-3-yl]oxy}-8-azaspiro[4.5]decane hydrochloride (51 mg). MS m/z: 301 [M+H]⁺.

Step 3: 8-[1-(2,2-difluoroethyl)-1H-pyrazolo[3,4-b]pyrazin-6-yl]-2-{[2-(trifluoromethyl)pyridin-3-yl]oxy}-8-azaspiro[4.5]decane: Followed the general procedure A using 6-chloro-1-(2,2-difluoroethyl)-1H-pyrazolo[3,4-b]pyrazine (17.8 mg, 81.7 μmol, 1.0 equiv) and 2-{[2-(trifluoromethyl)pyridin-3-yl]oxy}-8-azaspiro[4.5]decane hydrochloride (27.5 mg, 81.7 μmol, 1.0 equiv) as the starting materials to give 8-[1-(2,2-difluoroethyl)-1H-pyrazolo[3,4-b]pyrazin-6-yl]-2-{[2-(trifluoromethyl)pyridin-3-yl]oxy}-8-azaspiro[4.5]decane (23 mg, 58%) as a colorless oil. ¹H NMR (500 MHz, DMSO-d₆) δ 8.46 (s, 1H), 8.23 (dd, J=4.5, 1.2 Hz, 1H), 8.11 (s, 1H), 7.81-7.76 (m, 1H), 7.67 (dd, J=8.6, 4.5 Hz, 1H), 6.43 (tt, J=54.9, 3.8 Hz, 1H), 5.13 (dq, J=6.1, 2.9 Hz, 1H), 4.68 (td, J=15.1, 3.8 Hz, 2H), 3.85-3.75 (m, 2H), 3.75-3.64 (m, 2H), 2.15 (dtd, J=14.1, 8.3, 5.8 Hz, 1H), 2.05-1.96 (m, 1H), 1.89-1.50 (m, 8H). MS m/z: 469 [M+H]⁺.

### 7-[1-(2,2-difluoroethyl)-1H-pyrazolo[3,4-b]pyrazin-6-yl]-1-{[2-(trifluoromethyl)pyridin-3-yl]oxy}-7-azaspiro[3.5]nonane (87)

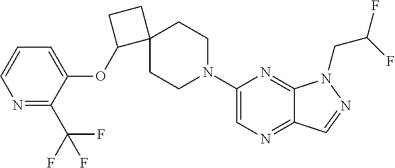 [SMILES: FC(F)Cn1ncc2ncc(N3CCC4(CCC4Oc4cccnc4C(F)(F)F)CC3)nc21]

Step 1: tert-butyl 1-{[2-(trifluoromethyl)pyridin-3-yl]oxy}-7-azaspiro[3.5]nonane-7-carboxylate: Followed the general procedure E using tert-butyl 1-hydroxy-7-azaspiro[3.5]nonane-7-carboxylate (50 mg, 207 μmol, 1.0 equiv) and 3-fluoro-2-(trifluoromethyl)pyridine (34.2 mg, 207 μmol, 1.0 equiv.) as the starting materials to give tert-butyl 1-{[2-(trifluoromethyl)pyridin-3-yl]oxy}-7-azaspiro[3.5]nonane-7-carboxylate (78 mg, 97%) as a colorless oil. MS m/z: 387 [M+H]

Step 2: 1-{[2-(trifluoromethyl)pyridin-3-yl]oxy}-7-azaspiro[3.5]nonane hydrochloride: Followed the general procedure B using tert-butyl 1-{[2-(trifluoromethyl)pyridin-3-yl]oxy}-7-azaspiro[3.5]nonane-7-carboxylate (78 mg) as the starting material to give 1-{[2-(trifluoromethyl)pyridin-3-yl]oxy}-7-azaspiro[3.5]nonane hydrochloride (75 mg). MS m/z: 287 [M+H]⁺.

Step 3: 7-[1-(2,2-difluoroethyl)-1H-pyrazolo[3,4-b]pyrazin-6-yl]-1-{[2-(trifluoromethyl)pyridin-3-yl]oxy}-7-azaspiro[3.5]nonane: Followed the general procedure A using 6-chloro-1-(2,2-difluoroethyl)-1H-pyrazolo[3,4-b]pyrazine (28.4 mg, 130 μmol, 1.0 equiv) and 1-{[2-(trifluoromethyl)pyridin-3-yl]oxy}-7-azaspiro[3.5]nonane hydrochloride (42 mg, 130 μmol, 1.0 equiv) as the starting materials to give 7-[1-(2,2-difluoroethyl)-1H-pyrazolo[3,4-b]pyrazin-6-yl]-1-{[2-(trifluoromethyl)pyridin-3-yl]oxy}-7-azaspiro[3.5]nonane (36 mg, 59%) as a colorless oil. ¹H NMR (500 MHz, DMSO-d₆) δ 8.49 (s, 1H), 8.24 (dd, J=3.9, 1.7 Hz, 1H), 8.12 (s, 1H), 7.69-7.62 (m, 2H), 6.43 (tt, J=54.9, 3.8 Hz, 1H), 4.79 (t, J=6.7 Hz, 1H), 4.69 (td, J=15.0, 3.8 Hz, 2H), 4.19 (dt, J=13.5, 4.8 Hz, 1H), 4.04 (dt, J=13.1, 4.6 Hz, 1H), 3.53 (ddd, J=13.2, 9.5, 3.5 Hz, 1H), 3.39 (ddd, J=13.2, 8.3, 4.6 Hz, 1H), 2.56-2.51 (m, 1H), 2.01-1.71 (m, 6H), 1.60 (dt, J=10.4, 8.1 Hz, 1H). MS m/z: 469 [M+H]⁺.

### 7-[1-(2,2-difluoroethyl)-1H-pyrazolo[3,4-b]pyrazin-6-yl]-2-{[2-(trifluoromethyl)pyridin-3-yl]oxy}-7-azaspiro[3.5]nonane (88)

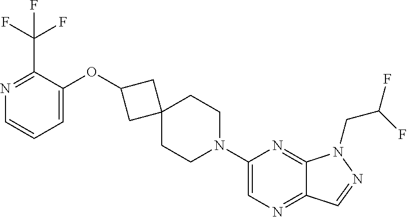 [SMILES: FC(F)Cn1ncc2ncc(N3CCC4(CC3)CC(Oc3cccnc3C(F)(F)F)C4)nc21]

Step 1: tert-butyl 2-{[2-(trifluoromethyl)pyridin-3-yl]oxy}-7-azaspiro[3.5]nonane-7-carboxylate: Followed the general procedure E using tert-butyl 2-hydroxy-7-azaspiro[3.5]nonane-7-carboxylate (50 mg, 207 μmol, 1.0 equiv) and 3-fluoro-2-(trifluoromethyl)pyridine (34.2 mg, 207 μmol, 1.0 equiv.) as the starting materials to give tert-butyl 1-{[2-(trifluoromethyl)pyridin-3-yl]oxy}-7-azaspiro[3.5]nonane-7-carboxylate (68 mg, 85%) as a colorless oil. MS m/z: 387 [M+H]⁺.

Step 2: 2-{[2-(trifluoromethyl)pyridin-3-yl]oxy}-7-azaspiro[3.5]nonane hydrochloride: Followed the general procedure B using tert-butyl 2-{[2-(trifluoromethyl)pyridin-3-yl]oxy}-7-azaspiro[3.5]nonane-7-carboxylate (68 mg) as the starting material to give 2-{[2-(trifluoromethyl)pyridin-3-yl]oxy}-7-azaspiro[3.5]nonane hydrochloride (65 mg). MS m/z: 287 [M+H]⁺.

Step 3: 7-[1-(2,2-difluoroethyl)-1H-pyrazolo[3,4-b]pyrazin-6-yl]-2-{[2-(trifluoromethyl)pyridin-3-yl]oxy}-7-azaspiro[3.5]nonane: Followed the general procedure A using 6-chloro-1-(2,2-difluoroethyl)-1H-pyrazolo[3,4-b]pyrazine (19.6 mg, 89.8 μmol, 1.0 equiv) and 2-{[2-(trifluoromethyl)pyridin-3-yl]oxy}-7-azaspiro[3.5]nonane hydrochloride (29 mg, 89.8 μmol, 1.0 equiv) as the starting materials to give 7-[l-(2,2-difluoroethyl)-1H-pyrazolo[3,4-b]pyrazin-6-yl]-2-{[2-(trifluoromethyl)pyridin-3-yl]oxy}-7-azaspiro[3.5]nonane (34 mg, 81%) as a colorless oil. ¹H NMR (500 MHz, DMSO-d₆) δ 8.47 (s, 1H), 8.26 (dd, J=4.5, 1.3 Hz, 1H), 8.12 (s, 1H), 7.67 (dd, J=8.6, 4.4 Hz, 1H), 7.63 (dd, J=8.7, 1.3 Hz, 1H), 6.43 (tt, J=54.9, 3.8 Hz, 1H), 5.02 (p, J=6.5 Hz, 1H), 4.69 (td, J=15.1, 3.8 Hz, 2H), 3.78-3.71 (m, 2H), 3.71-3.60 (m, 2H), 2.56-2.51 (m, 2H), 1.94-1.81 (m, 2H), 1.76-1.69 (m, 2H), 1.69-1.60 (m, 2H). MS m/z: 469 [M+H]⁺.

### 7-[1-(2,2-difluoroethyl)-1H-pyrazolo[3,4-b]pyrazin-6-yl]-2-({[2-(trifluoromethyl)pyridin-3-yl]oxy}methyl)-7-azaspiro[3.5]nonane (89)

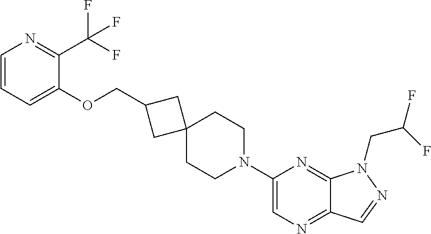 [SMILES: FC(F)Cn1ncc2ncc(N3CCC4(CC3)CC(COc3cccnc3C(F)(F)F)C4)nc21]

Step 1: tert-butyl 2-({[2-(trifluoromethyl)pyridin-3-yl]oxy}methyl)-7-azaspiro[3.5]nonane-7-carboxylate: Followed the general procedure E using tert-butyl 2-(hydroxymethyl)-7-azaspiro[3.5]nonane-7-carboxylate (50 mg, 196 μmol, 1.0 equiv) and 3-fluoro-2-(trifluoromethyl)pyridine (32.3 mg, 196 μmol, 1.0 equiv.) as the starting materials to give tert-butyl 2-({[2-(trifluoromethyl)pyridin-3-yl]oxy}methyl)-7-azaspiro[3.5]nonane-7-carboxylate (65 mg, 83%) as a colorless oil. MS m/z: 401 [M+H]⁺.

Step 2: 2-({[2-(trifluoromethyl)pyridin-3-yl]oxy}methyl)-7-azaspiro[3.5]nonane hydrochloride: Followed the general procedure B using tert-butyl 2-({[2-(trifluoromethyl)pyridin-3-yl]oxy}methyl)-7-azaspiro[3.5]nonane-7-carboxylate (65 mg) as the starting material to give 2-({[2-(trifluoromethyl)pyridin-3-yl]oxy}methyl)-7-azaspiro[3.5]nonane hydrochloride (50 mg). MS m/z: 301 [M+H]⁺.

Step 3: 7-[1-(2,2-difluoroethyl)-1H-pyrazolo[3,4-b]pyrazin-6-yl]-2-({[2-(trifluoromethyl)pyridin-3-yl]oxy}methyl)-7-azaspiro[3.5]nonane: Followed the general procedure A using 6-chloro-1-(2,2-difluoroethyl)-1H-pyrazolo[3,4-b]pyrazine (16.2 mg, 74.2 μmol, 1.0 equiv) and 2-({[2-(trifluoromethyl)pyridin-3-yl]oxy}methyl)-7-azaspiro[3.5]nonane hydrochloride (25 mg, 74.2 μmol, 1.0 equiv) as the starting materials to give 7-[1-(2,2-difluoroethyl)-1H-pyrazolo[3,4-b]pyrazin-6-yl]-2-({[2-(trifluoromethyl)pyridin-3-yl]oxy}methyl)-7-azaspiro[3.5]nonane (27 mg, 75%) as a colorless oil. ¹H NMR (500 MHz, DMSO-d₆) δ 8.46 (s, 1H), 8.25 (dd, J=4.5, 1.2 Hz, 1H), 8.11 (s, 1H), 7.80 (d, J=8.5 Hz, 1H), 7.69 (dd, J=8.6, 4.5 Hz, 1H), 6.43 (tt, J=54.9, 3.8 Hz, 1H), 4.68 (td, J=15.0, 3.8 Hz, 2H), 4.16 (d, J=5.5 Hz, 2H), 3.78-3.69 (m, 2H), 3.69-3.59 (m, 2H), 2.79 (tt, J=8.6, 5.5 Hz, 1H), 1.96 (ddd, J=12.5, 7.4, 3.0 Hz, 2H), 1.83-1.74 (m, 2H), 1.74-1.65 (m, 2H), 1.65-1.55 (m, 2H). MS m/z: 483 [M+H]⁺.

### 1-(2,2-difluoroethyl)-6-(2-((4-(trifluoromethyl)pyridin-2-yl)methyl)-2,7-diazaspiro[3.5]nonan-7-yl)-1H-pyrazolo[3,4-b]pyrazine (90)

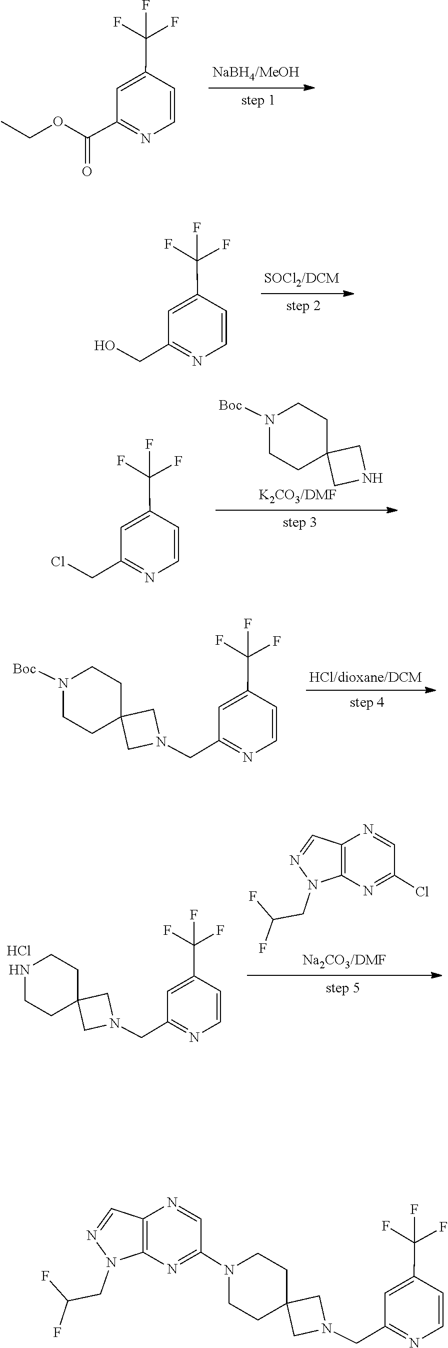

Step 1: (4-(trifluoromethyl)pyridin-2-yl)methanol: To a stirred solution of ethyl 4-(trifluoromethyl)pyridine-2-carboxylate (300 mg, 1.37 mmol, 1 equiv) in MeOH (3 mL) was added NaBH₄ (103 mg, 2.74 mmol, 2 equiv) at 0° C. The resulting mixture was stirred for 2 h at room temperature. The reaction was quenched by the addition of sat. NH₄Cl (aq.) (2 mL) at 0° C. The resulting mixture was extracted with EtOAc (3×10 mL). The combined organic layers were washed with brine (1×10 mL), dried over anhydrous Na₂SO4. After filtration, the filtrate was concentrated under reduced pressure. The residue was purified by silica gel column chromatography, eluted with PE/EtOAc (1:1) to afford [4-(trifluoromethyl)pyridin-2-yl]methanol (200 mg, 82.5%) as a colorless oil. MS m/z: 178 [M+H]⁺.

Step 2: 2-(chloromethyl)-4-(trifluoromethyl)pyridine: To a stirred solution of [4-(trifluoromethyl)pyridin-2-yl]methanol (100 mg, 0.565 mmol, 1 equiv) in DCM (1 mL) was added SOCl₂ (335 mg, 2.82 mmol, 5 equiv) dropwise at 0° C. The resulting mixture was stirred for 2 h at room temperature. The resulting mixture was concentrated under reduced pressure. The residue was purified by silica gel column chromatography, eluted with PE/EtOAc (5:1) to afford 2-(chloromethyl)-4-(trifluoromethyl)pyridine (80 mg, 72.4%) as a colorless oil. MS m/z: 196 [M+H]⁺.

Step 3: tert-butyl 2-{[4-(trifluoromethyl)pyridin-2-yl]methyl}-2,7-diazaspiro[3.5]nonane-7-carboxylate: Followed the general procedure A using 2-(chloromethyl)-4-(trifluoromethyl)pyridine (70 mg, 0.358 mmol, 1 equiv) and tert-butyl 2,7-diazaspiro[3.5]nonane-7-carboxylate (89.0 mg, 0.394 mmol, 1.1 equiv) as the starting material to afford tert-butyl 2-{[4-(trifluoromethyl)pyridin-2-yl]methyl}-2,7-diazaspiro[3.5]nonane-7-carboxylate (70 mg, 50.7%) as a white solid. MS m/z: 386 [M+H]⁺.

Step 4: 2-((4-(trifluoromethyl)pyridin-2-yl)methyl)-2,7-diazaspiro[3.5]nonane hydrochloride: Followed the general procedure B using tert-butyl 2-{[4-(trifluoromethyl)pyridin-2-yl]methyl}-2,7-diazaspiro[3.5]nonane-7-carboxylate (60 mg, 0.156 mmol, 1 equiv) as the starting material to afford the crude product 2-((4-(trifluoromethyl)pyridin-2-yl)methyl)-2,7-diazaspiro[3.5]nonane hydrochloride (60 mg) was directly used in next step without further purification MS m/z: 322 [M+H]⁺.

Step 5: 1-(2,2-difluoroethyl)-6-(2-((4-(trifluoromethyl)pyridin-2-yl)methyl)-2,7-diazaspiro[3.5]nonan-7-yl)-1H-pyrazolo[3,4-b]pyrazine: To a stirred solution of 2-((4-(trifluoromethyl)pyridin-2-yl)methyl)-2,7-diazaspiro[3.5]nonane hydrochloride (60 mg, 0.186 mmol, 1 equiv) and 6-chloro-1-(2,2-difluoroethyl)pyrazolo[3,4-b]pyrazine (40.7 mg, 0.186 mmol, 1 equiv) in DMF (2 mL) was added Na₂CO₃ (59.8 mg, 0.558 mmol, 3 equiv) at room temperature. The resulting mixture was stirred for 2 h at 100° C. The resulting mixture was diluted with water (20 mL) and extracted with EtOAc (15 mL×3). The combined organic layers were washed with brine (30 mL), dried over anhydrous Na₂SO₄. After filtration, the filtrate was concentrated to dryness under reduced pressure. The residue was purified by reverse flash chromatography with the following conditions: column, silica gel; mobile phase, MeCN in water, 10% to 50% gradient in 20 min; detector, UV 254 nm to afford 1-(2,2-difluoroethyl)-6-(2-((4-(trifluoromethyl)pyridin-2-yl)methyl)-2,7-diazaspiro[3.5]nonan-7-yl)-1H-pyrazolo[3,4-b]pyrazine (64.6 mg, 74.0%) as a off-white solid. ¹H NMR (400 MHz, DMSO-d₆) δ 8.79 (d, J=5.2 Hz, 1H), 8.46 (s, 1H), 8.11 (s, 1H), 7.65 (d, J=4.1 Hz, 2H), 6.59-6.27 (m, 1H), 4.74-4.63 (m, 2H), 3.86 (s, 2H), 3.76-3.67 (m, 4H), 3.14 (s, 4H), 1.86-1.75 (m, 4H). MS m/z: 468.2 [M+H]⁺.

### 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decan-3-one (91)

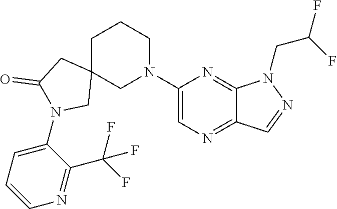 [SMILES: O=C1CC2(CCCN(c3cnc4cnn(CC(F)F)c4n3)C2)CN1c1cccnc1C(F)(F)F]

Step 1: tert-butyl 3-oxo-2-[2-(trifluoromethyl)pyridin-3-yl]-2,7-diazaspiro[4.5]decane-7-carboxylate: Followed the general procedure E using tert-butyl 3-oxo-2,7-diazaspiro[4.5]decane-7-carboxylate (50 mg, 197 μmol, 1.0 equiv) and 3-fluoro-2-(trifluoromethyl)pyridine (32.5 mg, 197 μmol, 1.0 equiv.) as the starting materials to give tert-butyl 3-oxo-2-[2-(trifluoromethyl)pyridin-3-yl]-2,7-diazaspiro[4.5]decane-7-carboxylate (60 mg, 76%) as a colorless oil. MS m/z: 400 [M+H]⁺.

Step 2: 2-[2-(trifluoromethyl)pyridin-3-yl]-2,7-diazaspiro[4.5]decan-3-one hydrochloride: Followed the general procedure B using tert-butyl 3-oxo-2-[2-(trifluoromethyl)pyridin-3-yl]-2,7-diazaspiro[4.5]decane-7-carboxylate (60 mg) as the starting material to give 2-[2-(trifluoromethyl)pyridin-3-yl]-2,7-diazaspiro[4.5]decan-3-one hydrochloride (50 mg). MS m/z: 300 [M+H]⁺.

Step 3: 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decan-3-one: Followed the general procedure A using 6-chloro-1-(2,2-difluoroethyl)-1H-pyrazolo[3,4-b]pyrazine (19.5 mg, 89.4 μmol, 1.0 equiv) and 2-[2-(trifluoromethyl)pyridin-3-yl]-2,7-diazaspiro[4.5]decan-3-one hydrochloride (30 mg, 89.4 μmol, 1.0 equiv) as the starting materials to give 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decan-3-one (29 mg, 67%) as a colorless oil. ¹H NMR (500 MHz, DMSO-d₆) δ 8.73 (dd, J=4.7, 1.5 Hz, 1H), 8.49 (s, 1H), 8.13 (s, 1H), 8.12-8.04 (m, 1H), 7.85 (dd, J=8.1, 4.6 Hz, 1H), 6.41 (tt, J=55.0, 4.0 Hz, 1H), 4.72-4.60 (m, 2H), 4.10-4.02 (m, 1H), 3.85 (dt, J=11.1, 5.2 Hz, 1H), 3.75-3.65 (m, 2H), 3.62 (d, J=9.4 Hz, 1H), 3.54 (d, J=9.4 Hz, 1H), 2.46-2.32 (m, 2H), 1.86-1.77 (m, 2H), 1.72-1.68 (m, 2H). MS m/z: 482 [M+H]⁺.

### 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decan-1-one (92)

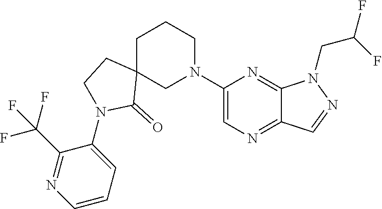 [SMILES: O=C1N(c2cccnc2C(F)(F)F)CCC12CCCN(c1cnc3cnn(CC(F)F)c3n1)C2]

Step 1: tert-butyl 1-oxo-2-[2-(trifluoromethyl)pyridin-3-yl]-2,7-diazaspiro[4.5]decane-7-carboxylate: Followed the general procedure E using tert-butyl 1-oxo-2,7-diazaspiro[4.5]decane-7-carboxylate (50 mg, 197 μmol, 1.0 equiv) and 3-fluoro-2-(trifluoromethyl)pyridine (32.5 mg, 197 μmol, 1.0 equiv.) as the starting materials to give tert-butyl 1-oxo-2-[2-(trifluoromethyl)pyridin-3-yl]-2,7-diazaspiro[4.5]decane-7-carboxylate (42 mg, 53%) as a colorless oil. MS m/z: 400 [M+H]⁺.

Step 2: 2-[2-(trifluoromethyl)pyridin-3-yl]-2,7-diazaspiro[4.5]decan-1-one hydrochloride: Followed the general procedure B using tert-butyl 1-oxo-2-[2-(trifluoromethyl)pyridin-3-yl]-2,7-diazaspiro[4.5]decane-7-carboxylate (42 mg) as the starting material to give 2-[2-(trifluoromethyl)pyridin-3-yl]-2,7-diazaspiro[4.5]decan-1-one hydrochloride (34 mg). MS m/z: 300 [M+H]⁺.

Step 3: 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decan-1-one: Followed the general procedure A using 6-chloro-1-(2,2-difluoroethyl)-1H-pyrazolo[3,4-b]pyrazine (22.8 mg, 104 μmol, 1.0 equiv) and 2-[2-(trifluoromethyl)pyridin-3-yl]-2,7-diazaspiro[4.5]decan-1-one hydrochloride (35 mg, 104 μmol, 1.0 equiv) as the starting materials to give 7-[1-(2,2-difluoroethyl)-1H-pyrazolo[3,4-b]pyrazin-6-yl]-2-[2-(trifluoromethyl)pyridin-3-yl]-2,7-diazaspiro[4.5]decan-1-one (42 mg, 84%) as a colorless oil. ¹H NMR (500 MHz, DMSO-d₆) δ 8.76 (dd, J=4.7, 1.5 Hz, 1H), 8.54 (s, 1H), 8.16 (dd, J=8.1, 1.5 Hz, 1H), 8.13 (s, 1H), 7.90 (dd, J=8.1, 4.6 Hz, 1H), 6.43 (tt, J=54.9, 3.8 Hz, 1H), 4.75-4.60 (m, 3H), 4.54-4.42 (m, 1H), 3.83 (dt, J=9.4, 7.5 Hz, 1H), 3.62 (td, J=9.0, 3.9 Hz, 1H), 3.24-3.14 (m, 2H), 2.14 (ddd, J=12.8, 7.7, 4.0 Hz, 1H), 2.03-1.88 (m, 3H), 1.82 (dt, J=13.4, 3.6 Hz, 1H), 1.77-1.57 (m, 2H). MS m/z: 482 [M+H]⁺.

### 1-(2,2-difluoroethyl)-6-(2-((2-(trifluoromethyl)pyridin-3-yl)methyl)-2,7-diazaspiro[3.5]nonan-7-yl)-1H-pyrazolo[3,4-b]pyrazine (93)

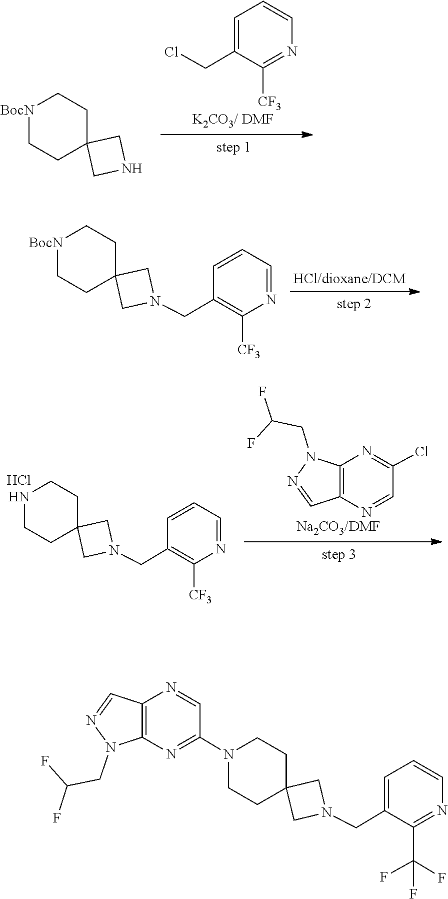

Step 1: tert-butyl 2-((2-(trifluoromethyl)pyridin-3-yl)methyl)-2,7-diazaspiro[3.5]nonane-7-carboxylate: Followed the general procedure A using tert-butyl 2,7-diazaspiro[3.5]nonane-7-carboxylate (90.0 mg, 0.398 mmol, 1.00 equiv) and 3-(chloromethyl)-2-(trifluoromethyl)pyridine (93.33 mg, 0.478 mmol, 1.2 equiv) as the starting materials to afford tert-butyl 2-((2-(trifluoromethyl)pyridin-3-yl)methyl)-2,7-diazaspiro[3.5]nonane-7-carboxylate (60.0 mg, 39.15%) as a yellow oil. MS m/z: 330 [M−tBu+H]⁺.

Step 2: 2-((2-(trifluoromethyl)pyridin-3-yl)methyl)-2,7-diazaspiro[3.5]nonane hydrochloride: Followed the general procedure B using tert-butyl 2-((2-(trifluoromethyl)pyridin-3-yl)methyl)-2,7-diazaspiro[3.5]nonane-7-carboxylate (60.0 mg, 0.156 mmol, 1.00 equiv) as the starting materials to afford the crude product 2-((2-(trifluoromethyl)pyridin-3-yl)methyl)-2,7-diazaspiro[3.5]nonane hydrochloride (50 mg). MS m/z: 286 [M+H]⁺.

Step 3: 1-(2,2-difluoroethyl)-6-(2-((2-(trifluoromethyl)pyridin-3-yl)methyl)-2,7-diazaspiro[3.5]nonan-7-yl)-1H-pyrazolo[3,4-b]pyrazine: To a stirred solution of 2-((2-(trifluoromethyl)pyridin-3-yl)methyl)-2,7-diazaspiro[3.5]nonane hydrochloride (50 mg, 0.156 mmol, 1.00 equiv), 6-chloro-1-(2,2-difluoroethyl)-1H-pyrazolo[3,4-b]pyrazine (44.7 mg, 0.175 mmol, 1.12 equiv) and Na₂CO₃ (55.72 mg, 0.525 mmol, 3.00 equiv) in DMF (1.00 mL) at room temperature. The resulting mixture was stirred for 2 h at 100° C. The resulting mixture was diluted with water (20 mL) and extracted with EtOAc (15 mL×3). The combined organic layers were washed with brine (30 mL), dried over anhydrous Na₂SO₄. After filtration, the filtrate was concentrated to dryness under reduced pressure. The residue was purified by reversed phase Combi-flash chromatography with the following conditions (column, C18 gel; mobile phase, B phase: MeCN, A phase: water; 5% to 100% B gradient in 15 min; detector: UV 254/220 nm). The pure fraction was concentrated under vacuum to afford 1-(2,2-difluoroethyl)-6-(2-((2-(trifluoromethyl)pyridin-3-yl)methyl)-2,7-diazaspiro[3.5]nonan-7-yl)-1H-pyrazolo[3,4-b]pyrazine (18 mg, 24.6%) as a yellow semi-solid. ¹H NMR (400 MHz, DMSO-d₆) δ 8.60 (d, J=4.4 Hz, 1H), 8.46 (s, 1H), 8.17-8.09 (m, 2H), 7.75-7.67 (m, 1H), 6.61-6.27 (m, 1H), 4.75-4.62 (m, 2H), 3.83 (s, 2H), 3.75-3.67 (m, 4H), 3.10 (s, 4H), 1.81 (t, J=5.6 Hz, 4H). MS m/z: 468.1 [M+H]⁺.

### 1-(2,2-difluoroethyl)-6-(2-((6-(trifluoromethyl)pyridin-2-yl)methyl)-2,7-diazaspiro[3.5]nonan-7-yl)-1H-pyrazolo[3,4-b]pyrazine (94)

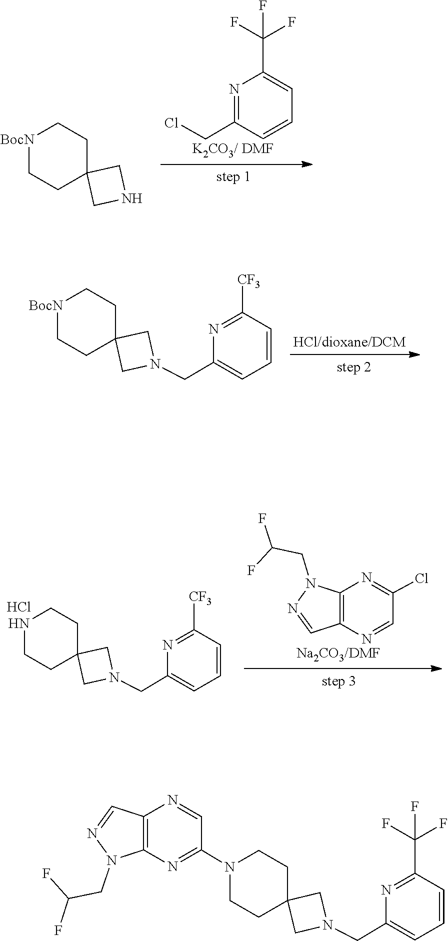

Step 1: tert-butyl 2-((6-(trifluoromethyl)pyridin-2-yl)methyl)-2,7-diazaspiro[3.5]nonane-7-carboxylate: Followed the general procedure A using tert-butyl 2,7-diazaspiro[3.5]nonane-7-carboxylate (120 mg, 0.530 mmol, 1.00 equiv) and 2-(chloromethyl)-6-(trifluoromethyl)pyridine (124.43 mg, 0.636 mmol, 1.2 equiv) as the starting materials to afford tert-butyl 2-((6-(trifluoromethyl)pyridin-2-yl)methyl)-2,7-diazaspiro[3.5]nonane-7-carboxylate (100 mg, 48.93%) as a yellow oil. MS m/z: 330 [M−tBu+H]⁺.

Step 2: 2-((6-(trifluoromethyl)pyridin-2-yl)methyl)-2,7-diazaspiro[3.5]nonane: Followed the general procedure B using tert-butyl 2-((6-(trifluoromethyl)pyridin-2-yl)methyl)-2,7-diazaspiro[3.5]nonane-7-carboxylate (100 mg, 0.259 mmol, 1.00 equiv) as the starting materials to afford the crude product 2-((6-(trifluoromethyl)pyridin-2-yl)methyl)-2,7-diazaspiro[3.5]nonane (90.0 mg). MS m/z: 286 [M+H]⁺.

Step 3: 1-(2,2-difluoroethyl)-6-(2-((6-(trifluoromethyl)pyridin-2-yl)methyl)-2,7-diaza spiro[3.5]nonan-7-yl)-1H-pyrazolo[3,4-b]pyrazine: To a stirred solution of 2-((6-(trifluoromethyl)pyridin-2-yl)methyl)-2,7-diaza spiro[3.5]nonane hydrochloride (50.0 mg, 0.155 mmol, 1.00 equiv), 6-chloro-1-(2,2-difluoroethyl)-1H-pyrazolo[3,4-b]pyrazine (33.97 mg, 0.155 mmol, 1.00 equiv) and Na₂CO₃ (49.41 mg, 0.465 mmol, 3.00 equiv) in DMF (1.00 mL) at room temperature. The resulting mixture was stirred for 2 h at 100° C. The resulting mixture was diluted with water (20 mL) and extracted with EtOAc (15 mL×3). The combined organic layers were washed with brine (30 mL), dried over anhydrous Na₂SO₄. After filtration, the filtrate was concentrated to dryness under reduced pressure. The residue was purified by reversed phase Combi-flash chromatography with the following conditions (column, C18 gel; mobile phase, B phase: MeCN, A phase: water; 5% to 100% B gradient in 15 min; detector: UV 254/220 nm). The pure fraction was concentrated under vacuum to afford 1-(2,2-difluoroethyl)-6-(2-((6-(trifluoromethyl) pyridin-2-yl) methyl)-2,7-diazaspiro[3.5]nonan-7-yl)-1H-pyrazolo[3,4-b]pyrazine (29 mg, 37.61%) as an off-white oil. ¹H NMR (400 MHz, DMSO-d₆) δ 8.46 (s, 1H), 8.11 (s, 1H), 8.10-8.04 (m, 1H), 7.78 (d, J=7.7 Hz, 1H), 7.70 (d, J=8.0 Hz, 1H), 6.64-6.12 (m, −1H), 4.75-4.62 (m, 2H), 3.83 (s, 2H), 3.71 (t, J=5.6 Hz, 4H), 1.80 (s, 4H). MS m/z: 468.2 [M+H]⁺.

### 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decan-3-one (95)

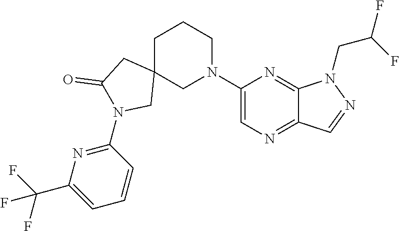 [SMILES: O=C1CC2(CCCN(c3cnc4cnn(CC(F)F)c4n3)C2)CN1c1cccc(C(F)(F)F)n1]

Step 1: tert-butyl 3-oxo-2-[6-(trifluoromethyl)pyridin-2-yl]-2,7-diazaspiro[4.5]decane-7-carboxylate: Followed the general procedure D using tert-butyl 3-oxo-2,7-diazaspiro[4.5]decane-7-carboxylate (100 mg, 393 μmol, 1.0 equiv) and 2-bromo-6-(trifluoromethyl)pyridine (88.9 mg, 393 μmol, 1.0 equiv.) as the starting materials to give tert-butyl 3-oxo-2-[6-(trifluoromethyl)pyridin-2-yl]-2,7-diazaspiro[4.5]decane-7-carboxylate (144 mg, 92%) as a colorless oil. MS m/z: 400 [M+H]⁺.

Step 2: 2-[6-(trifluoromethyl)pyridin-2-yl]-2,7-diazaspiro[4.5]decan-3-one hydrochloride: Followed the general procedure B using tert-butyl 3-oxo-2-[6-(trifluoromethyl)pyridin-2-yl]-2,7-diazaspiro[4.5]decane-7-carboxylate (142 mg) as the starting material to give 2-[6-(trifluoromethyl)pyridin-2-yl]-2,7-diazaspiro[4.5]decan-3-one hydrochloride (110 mg). MS m/z: 300 [M+H]⁺.

Step 3: 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decan-3-one: Followed the general procedure A using 6-chloro-1-(2,2-difluoroethyl)-1H-pyrazolo[3,4-b]pyrazine (15 mg, 68.6 μmol, 1.0 equiv) and 2-[6-(trifluoromethyl)pyridin-2-yl]-2,7-diazaspiro[4.5]decan-3-one hydrochloride (23 mg, 68.6 μmol, 1.0 equiv) as the starting materials to give 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decan-3-one (22 mg, 67%) as a colorless oil. ¹H NMR (500 MHz, DMSO-d₆) δ 8.61 (d, J=8.6 Hz, 1H), 8.49 (s, 1H), 8.16-8.00 (m, 2H), 7.66-7.58 (m, 1H), 6.33 (tt, J=55.0, 3.9 Hz, 1H), 4.53 (tdd, J=15.0, 7.4, 4.0 Hz, 2H), 3.95-3.82 (m, 3H), 3.80-3.61 (m, 3H), 2.62-2.52 (m, 2H), 1.89-1.76 (m, 2H), 1.72-1.67 (m, 2H). MS m/z: 482 [M+H]⁺.

### 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decan-1-one (96)

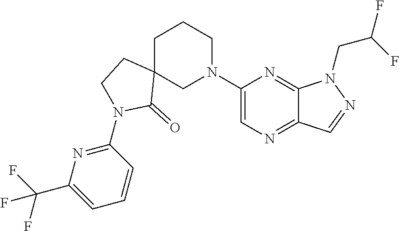 [SMILES: O=C1N(c2cccc(C(F)(F)F)n2)CCC12CCCN(c1cnc3cnn(CC(F)F)c3n1)C2]

Step 1: tert-butyl 1-oxo-2-[6-(trifluoromethyl)pyridin-2-yl]-2,7-diazaspiro[4.5]decane-7-carboxylate: Followed the general procedure D using tert-butyl 1-oxo-2,7-diazaspiro[4.5]decane-7-carboxylate (100 mg, 393 μmol, 1.0 equiv) and 2-bromo-6-(trifluoromethyl)pyridine (88.9 mg, 393 μmol, 1.0 equiv.) as the starting materials to give tert-butyl 1-oxo-2-[6-(trifluoromethyl)pyridin-2-yl]-2,7-diazaspiro[4.5]decane-7-carboxylate (45 mg, 29%) as a colorless oil. MS m/z: 400 [M+H]⁺.

Step 2: 2-[6-(trifluoromethyl)pyridin-2-yl]-2,7-diazaspiro[4.5]decan-1-one hydrochloride: Followed the general procedure B using tert-butyl 1-oxo-2-[6-(trifluoromethyl)pyridin-2-yl]-2,7-diazaspiro[4.5]decane-7-carboxylate (45 mg) as the starting material to give 2-[6-(trifluoromethyl)pyridin-2-yl]-2,7-diazaspiro[4.5]decan-1-one hydrochloride (40 mg). MS m/z: 300 [M+H]⁺.

Step 3: 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decan-1-one: Followed the general procedure A using 6-chloro-1-(2,2-difluoroethyl)-1H-pyrazolo[3,4-b]pyrazine (15 mg, 68.6 μmol, 1.0 equiv) and 2-[6-(trifluoromethyl)pyridin-2-yl]-2,7-diazaspiro[4.5]decan-1-one hydrochloride (23 mg, 68.6 μmol, 1.0 equiv) as the starting materials to give 7-[1-(2,2-difluoroethyl)-1H-pyrazolo[3,4-b]pyrazin-6-yl]-2-[6-(trifluoromethyl)pyridin-2-yl]-2,7-diazaspiro[4.5]decan-1-one (22 mg, 67%) as a colorless oil. ¹H NMR (500 MHz, DMSO-d₆) δ 8.59 (d, J=8.6 Hz, 1H), 8.53 (s, 1H), 8.15-8.07 (m, 2H), 7.66 (d, J=7.5 Hz, 1H), 6.39 (tt, J=54.9, 3.8 Hz, 1H), 4.65 (tdd, J=15.1, 3.9, 1.8 Hz, 2H), 4.56 (d, J=13.4 Hz, 1H), 4.45 (d, J=13.4 Hz, 1H), 4.08-3.94 (m, 2H), 3.25 (ddd, J=13.9, 11.5, 2.9 Hz, 2H), 2.07 (ddd, J=12.8, 8.0, 4.8 Hz, 1H), 1.98-1.88 (m, 2H), 1.88-1.75 (m, 2H), 1.63 (q, J=12.0 Hz, 1H). MS m/z: 482 [M+H]⁺.

### 7-[1-(2,2-difluoroethyl)-1H-pyrazolo[3,4-b]pyrazin-6-yl]-2-[4-(trifluoromethyl)pyridin-2-yl]-2,7-diazaspiro[4.5]decan-3-one (97)

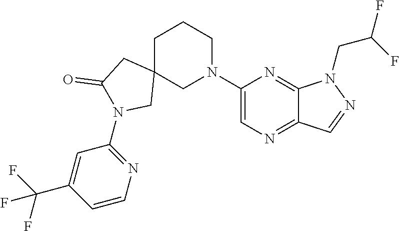 [SMILES: O=C1CC2(CCCN(c3cnc4cnn(CC(F)F)c4n3)C2)CN1c1cc(C(F)(F)F)ccn1]

Step 1: tert-butyl 3-oxo-2-[4-(trifluoromethyl)pyridin-2-yl]-2,7-diazaspiro[4.5]decane-7-carboxylate: Followed the general procedure D using tert-butyl 3-oxo-2,7-diazaspiro[4.5]decane-7-carboxylate (100 mg, 393 μmol, 1.0 equiv) and 2-bromo-4-(trifluoromethyl)pyridine (88.9 mg, 393 μmol, 1.0 equiv.) as the starting materials to give tert-butyl 3-oxo-2-[4-(trifluoromethyl)pyridin-2-yl]-2,7-diazaspiro[4.5]decane-7-carboxylate (92 mg, 59%) as a colorless oil. MS m/z: 400 [M+H]⁺.

Step 2: 2-[4-(trifluoromethyl)pyridin-2-yl]-2,7-diazaspiro[4.5]decan-3-one hydrochloride: Followed the general procedure B using tert-butyl 3-oxo-2-[4-(trifluoromethyl)pyridin-2-yl]-2,7-diazaspiro[4.5]decane-7-carboxylate (92 mg) as the starting material to give 2-[4-(trifluoromethyl)pyridin-2-yl]-2,7-diazaspiro[4.5]decan-3-one hydrochloride (80 mg). MS m/z: 300 [M+H]⁺.

Step 3: 7-[1-(2,2-difluoroethyl)-1H-pyrazolo[3,4-b]pyrazin-6-yl]-2-[4-(trifluoromethyl)pyridin-2-yl]-2,7-diazaspiro[4.5]decan-3-one: Followed the general procedure A using 6-chloro-1-(2,2-difluoroethyl)-1H-pyrazolo[3,4-b]pyrazine (15 mg, 68.6 μmol, 1.0 equiv) and 2-[4-(trifluoromethyl)pyridin-2-yl]-2,7-diazaspiro[4.5]decan-3-one hydrochloride (23 mg, 68.6 μmol, 1.0 equiv) as the starting materials to give 7-[1-(2,2-difluoroethyl)-1H-pyrazolo[3,4-b]pyrazin-6-yl]-2-[4-(trifluoromethyl)pyridin-2-yl]-2,7-diazaspiro[4.5]decan-3-one (18 mg, 54%) as a colorless oil. ¹H NMR (500 MHz, DMSO-d₆) δ 8.68 (dd, J=1.6, 0.9 Hz, 1H), 8.57 (d, J=5.2 Hz, 1H), 8.49 (s, 1H), 8.10 (s, 1H), 7.50 (dd, J=5.3, 1.6 Hz, 1H), 6.29 (tt, J=55.0, 4.0 Hz, 1H), 4.51 (tdd, J=14.9, 8.6, 4.0 Hz, 2H), 3.97 (dd, J=12.2, 3.7 Hz, 2H), 3.90 (dt, J=13.1, 5.3 Hz, 1H), 3.75 (d, J=11.2 Hz, 1H), 3.72-3.63 (m, 2H), 2.66-2.52 (m, 2H), 1.90-1.73 (m, 2H), 1.69 (p, J=5.8 Hz, 2H). MS m/z: 482 [M+H]⁺.

### 7-[1-(2,2-difluoroethyl)-1H-pyrazolo[3,4-b]pyrazin-6-yl]-2-[4-(trifluoromethyl)pyridin-2-yl]-2,7-diazaspiro[4.5]decan-1-one (98)

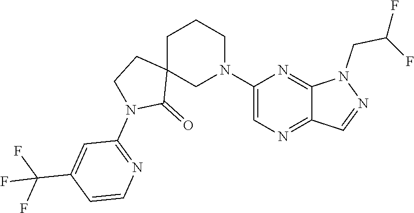 [SMILES: O=C1N(c2cc(C(F)(F)F)ccn2)CCC12CCCN(c1cnc3cnn(CC(F)F)c3n1)C2]

Step 1: tert-butyl 1-oxo-2-[4-(trifluoromethyl)pyridin-2-yl]-2,7-diazaspiro[4.5]decane-7-carboxylate: Followed the general procedure D using tert-butyl 1-oxo-2,7-diazaspiro[4.5]decane-7-carboxylate (100 mg, 393 μmol, 1.0 equiv) and 2-bromo-4-(trifluoromethyl)pyridine (88.9 mg, 393 μmol, 1.0 equiv.) as the starting materials to give tert-butyl 1-oxo-2-[4-(trifluoromethyl)pyridin-2-yl]-2,7-diazaspiro[4.5]decane-7-carboxylate (76 mg, 48%) as a colorless oil. MS m/z: 400 [M+H]⁺.

Step 2: 2-[4-(trifluoromethyl)pyridin-2-yl]-2,7-diazaspiro[4.5]decan-1-one hydrochloride: Followed the general procedure B using tert-butyl 1-oxo-2-[4-(trifluoromethyl)pyridin-2-yl]-2,7-diazaspiro[4.5]decane-7-carboxylate (76 mg) as the starting material to give 2-[4-(trifluoromethyl)pyridin-2-yl]-2,7-diazaspiro[4.5]decan-1-one hydrochloride (65 mg). MS m/z: 300 [M+H]⁺.

Step 3: 7-[1-(2,2-difluoroethyl)-1H-pyrazolo[3,4-b]pyrazin-6-yl]-2-[4-(trifluoromethyl)pyridin-2-yl]-2,7-diazaspiro[4.5]decan-1-one: Followed the general procedure A using 6-chloro-1-(2,2-difluoroethyl)-1H-pyrazolo[3,4-b]pyrazine (15 mg, 68.6 μmol, 1.0 equiv) and 2-[4-(trifluoromethyl)pyridin-2-yl]-2,7-diazaspiro[4.5]decan-1-one hydrochloride (23 mg, 68.6 μmol, 1.0 equiv) as the starting materials to give 7-[1-(2,2-difluoroethyl)-1H-pyrazolo[3,4-b]pyrazin-6-yl]-2-[4-(trifluoromethyl)pyridin-2-yl]-2,7-diazaspiro[4.5]decan-1-one (17 mg, 51%) as a colorless oil. ¹H NMR (500 MHz, DMSO-d₆) δ 8.71 (d, J=5.2 Hz, 1H), 8.66 (dd, J=1.8, 1.0 Hz, 1H), 8.52 (s, 1H), 8.12 (s, 1H), 7.54 (dd, J=5.0, 1.6 Hz, 1H), 6.39 (tt, J=54.9, 3.8 Hz, 1H), 4.71-4.61 (m, 2H), 4.57 (d, J=13.4 Hz, 1H), 4.50-4.40 (m, 1H), 4.10-3.98 (m, 2H), 3.33-3.20 (m, 2H), 2.07 (ddd, J=12.8, 7.7, 5.2 Hz, 1H), 2.01-1.88 (m, 2H), 1.82 (ddt, J=27.3, 12.9, 3.9 Hz, 2H), 1.70-1.53 (m, 1H). MS m/z: 482 [M+H]⁺.

### 2-[1-(2,2-difluoroethyl)-1H-pyrazolo[3,4-b]pyrazin-6-yl]-8-[6-(trifluoromethyl)pyridin-2-yl]-2,8-diazaspiro[4.5]decan-1-one (99)

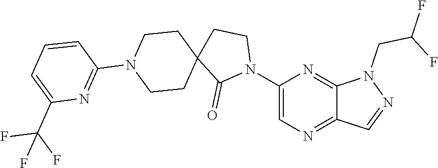 [SMILES: O=C1N(c2cnc3cnn(CC(F)F)c3n2)CCC12CCN(c1cccc(C(F)(F)F)n1)CC2]

Step 1: tert-butyl 1-oxo-2-[4-(trifluoromethyl)pyridin-2-yl]-2,7-diazaspiro[4.5]decane-7-carboxylate: Followed the general procedure A using 2,8-diazaspiro[4.5]decan-1-one hydrochloride (100 mg, 524 μmol, 1.0 equiv) and 2-bromo-6-(trifluoromethyl)pyridine (119 mg, 524 μmol, 1.0 equiv.) as the starting materials to give 8-[6-(trifluoromethyl)pyridin-2-yl]-2,8-diazaspiro[4.5]decan-1-one (109 mg, 69%) as a white solid. MS m/z: 300 [M+H]⁺.

Step 2: 2-[1-(2,2-difluoroethyl)-1H-pyrazolo[3,4-b]pyrazin-6-yl]-8-[6-(trifluoromethyl)pyridin-2-yl]-2,8-diazaspiro[4.5]decan-1-one: Followed the general procedure D using 6-chloro-1-(2,2-difluoroethyl)-1H-pyrazolo[3,4-b]pyrazine (25 mg, 114 μmol, 1.2 equiv) and 8-[6-(trifluoromethyl)pyridin-2-yl]-2,8-diazaspiro[4.5]decan-1-one (28.5 mg, 95.3 μmol, 1.0 equiv) as the starting materials to give 2-[1-(2,2-difluoroethyl)-1H-pyrazolo[3,4-b]pyrazin-6-yl]-8-[6-(trifluoromethyl)pyridin-2-yl]-2,8-diazaspiro[4.5]decan-1-one (34 mg, 74%) as a colorless oil. ¹H NMR (500 MHz, DMSO-d₆) δ 9.66 (s, 1H), 8.49 (s, 1H), 7.80-7.70 (m, 1H), 7.20 (d, J=8.8 Hz, 1H), 7.04 (d, J=7.3 Hz, 1H), 6.52 (tt, J=54.6, 3.6 Hz, 1H), 4.88 (td, J=15.1, 3.6 Hz, 2H), 4.25 (dt, J=13.5, 4.1 Hz, 2H), 4.10 (dd, J=7.8, 6.4 Hz, 2H), 3.20 (ddd, J=13.9, 11.4, 3.0 Hz, 2H), 2.30-2.22 (m, 2H), 1.80 (ddd, J=13.2, 11.3, 4.2 Hz, 2H), 1.74-1.65 (m, 2H). MS m/z: 482 [M+H]⁺.

### 2-[1-(2,2-difluoroethyl)-1H-pyrazolo[3,4-b]pyrazin-6-yl]-8-[6-(trifluoromethyl)pyridin-2-yl]-2,8-diazaspiro[4.5]decan-3-one (100)

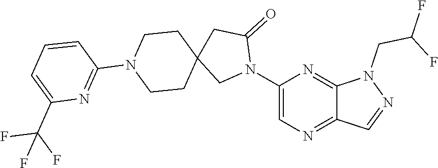 [SMILES: O=C1CC2(CCN(c3cccc(C(F)(F)F)n3)CC2)CN1c1cnc2cnn(CC(F)F)c2n1]

Step 1: tert-butyl 1-oxo-2-[4-(trifluoromethyl)pyridin-2-yl]-2,7-diazaspiro[4.5]decane-7-carboxylate: Followed the general procedure A using 2,8-diazaspiro[4.5]decan-3-one hydrochloride (100 mg, 524 μmol, 1.0 equiv) and 2-bromo-6-(trifluoromethyl)pyridine (119 mg, 524 μmol, 1.0 equiv.) as the starting materials to give 8-[6-(trifluoromethyl)pyridin-2-yl]-2,8-diazaspiro[4.5]decan-3-one (81 mg, 51%) as a white solid. MS m/z: 300 [M+H]⁺.

Step 2: 2-[1-(2,2-difluoroethyl)-1H-pyrazolo[3,4-b]pyrazin-6-yl]-8-[6-(trifluoromethyl)pyridin-2-yl]-2,8-diazaspiro[4.5]decan-3-one: Followed the general procedure D using 6-chloro-1-(2,2-difluoroethyl)-1H-pyrazolo[3,4-b]pyrazine (25 mg, 114 μmol, 1.2 equiv) and 8-[6-(trifluoromethyl)pyridin-2-yl]-2,8-diazaspiro[4.5]decan-3-one (28.5 mg, 95.3 μmol, 1.0 equiv) as the starting materials to give 2-[1-(2,2-difluoroethyl)-1H-pyrazolo[3,4-b]pyrazin-6-yl]-8-[6-(trifluoromethyl)pyridin-2-yl]-2,8-diazaspiro[4.5]decan-3-one (32 mg, 70%) as a colorless oil. ¹H NMR (500 MHz, DMSO-d₆) δ 9.65 (d, J=1.5 Hz, 1H), 8.53-8.43 (m, 1H), 7.74 (t, J=7.6 Hz, 1H), 7.18 (dd, J=8.9, 2.4 Hz, 1H), 7.02 (dd, J=7.1, 2.1 Hz, 1H), 6.51 (tt, J=54.6, 3.7 Hz, 1H), 4.87 (td, J=15.1, 3.7 Hz, 2H), 3.97 (s, 2H), 3.84 (dt, J=13.6, 5.0 Hz, 2H), 3.49 (dt, J=13.1, 6.1 Hz, 2H), 2.75 (s, 2H), 1.73 (t, J=5.6 Hz, 4H). MS m/z: 482 [M+H]⁺.

### 8-[1-(2,2-difluoroethyl)-1H-pyrazolo[3,4-b]pyrazin-6-yl]-2-[6-(trifluoromethyl)pyridin-2-yl]-2,8-diazaspiro[4.5]decan-3-one (101)

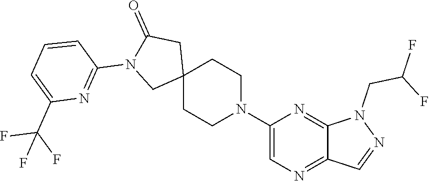 [SMILES: O=C1CC2(CCN(c3cnc4cnn(CC(F)F)c4n3)CC2)CN1c1cccc(C(F)(F)F)n1]

Followed the general procedure D using 8-[1-(2,2-difluoroethyl)-1H-pyrazolo[3,4-b]pyrazin-6-yl]-2,8-diazaspiro[4.5]decan-3-one (25 mg, 74.3 μmol, 1.0 equiv) and 2-bromo-6-(trifluoromethyl)pyridine (16.8 mg, 74.3 μmol, 1.0 equiv) as the starting materials to give 8-[1-(2,2-difluoroethyl)-1H-pyrazolo[3,4-b]pyrazin-6-yl]-2-[6-(trifluoromethyl)pyridin-2-yl]-2,8-diazaspiro[4.5]decan-3-one (28 mg, 78%) as a white solid. ¹H NMR (500 MHz, DMSO-d₆) δ 8.63-8.54 (m, 1H), 8.50 (s, 1H), 8.14 (s, 1H), 8.12-8.08 (m, 1H), 7.68-7.61 (m, 1H), 6.44 (tt, J=54.9, 3.8 Hz, 1H), 4.70 (td, J=15.0, 3.8 Hz, 2H), 4.07 (dt, J=13.9, 4.8 Hz, 2H), 3.88 (s, 2H), 3.57 (ddd, J=13.3, 8.6, 4.0 Hz, 2H), 2.72 (s, 2H), 1.87-1.69 (m, 4H). MS m/z: 482 [M+H]⁺.

### 8-[1-(2,2-difluoroethyl)-1H-pyrazolo[3,4-b]pyrazin-6-yl]-2-[6-(trifluoromethyl)pyridin-2-yl]-2,8-diazaspiro[4.5]decan-1-one (102)

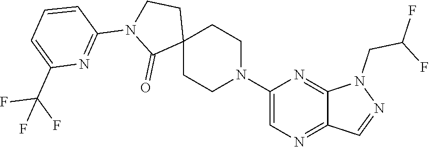 [SMILES: O=C1N(c2cccc(C(F)(F)F)n2)CCC12CCN(c1cnc3cnn(CC(F)F)c3n1)CC2]

Followed the general procedure D using 8-[1-(2,2-difluoroethyl)-1H-pyrazolo[3,4-b]pyrazin-6-yl]-2,8-diazaspiro[4.5]decan-1-one (25 mg, 74.3 μmol, 1.0 equiv.) and 2-bromo-6-(trifluoromethyl)pyridine (16.8 mg, 74.3 μmol, 1.0 equiv.) as the starting materials to give 8-[1-(2,2-difluoroethyl)-1H-pyrazolo[3,4-b]pyrazin-6-yl]-2-[6-(trifluoromethyl)pyridin-2-yl]-2,8-diazaspiro[4.5]decan-1-one (30 mg, 84%) as a colorless oil. ¹H NMR (500 MHz, DMSO-d₆) δ 8.63-8.56 (m, 1H), 8.52 (s, 1H), 8.15 (s, 1H), 8.10 (ddd, J=8.7, 7.7, 0.8 Hz, 1H), 7.65 (dd, J=7.5, 0.7 Hz, 1H), 6.45 (tt, J=54.9, 3.8 Hz, 1H), 4.71 (td, J=15.0, 3.8 Hz, 2H), 4.44 (dt, J=13.7, 4.1 Hz, 2H), 4.07-3.98 (m, 2H), 3.36-3.31 (m, 2H, overlapped with water peak), 2.21 (t, J=7.0 Hz, 2H), 1.83 (ddd, J=13.3, 11.4, 4.2 Hz, 2H), 1.79-1.66 (m, 2H). MS m/z: 482 [M+H]⁺.

### 2-{2-benzyl-2H-pyrazolo[3,4-b]pyrazin-6-yl}-6-[2-(trifluoromethyl)pyridin-4-yl]-2,6-diazaspiro[3.5]nonane (103)

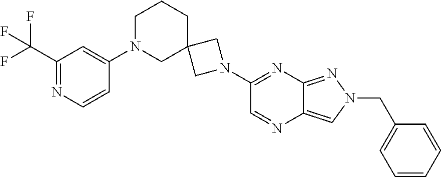

Followed the general procedure A using 2-benzyl-6-chloro-2H-pyrazolo[3,4-b]pyrazine (15.9 mg, 65 μmol, 1.0 equiv.) and 6-[2-(trifluoromethyl)pyridin-4-yl]-2,6-diazaspiro[3.5]nonane hydrochloride (20 mg, 65 μmol, 1.0 equiv.) as the starting materials to give 2-{2-benzyl-2H-pyrazolo[3,4-b]pyrazin-6-yl}-6-[2-(trifluoromethyl)pyridin-4-yl]-2,6-diazaspiro[3.5]nonane (20 mg, 64%) as a colorless oil. ¹H NMR (500 MHz, DMSO-d₆) δ 8.24 (d, J=6.0 Hz, 1H), 8.08 (s, 1H), 7.92 (s, 1H), 7.34-7.24 (m, 4H), 7.24-7.20 (m, 2H), 7.14 (dd, J=6.1, 2.6 Hz, 1H), 5.43 (s, 2H), 3.97-3.82 (m, 4H), 3.69 (s, 2H), 3.40 (t, J=5.6 Hz, 2H), 1.94-1.86 (m, 2H), 1.69-1.59 (m, 2H). MS m/z: 480 [M+H]⁺.

### 2-{1-benzyl-1H-pyrazolo[3,4-b]pyrazin-6-yl}-6-[2-(trifluoromethyl)pyridin-4-yl]-2,6-diazaspiro[3.5]nonane (104)

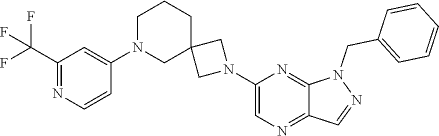 [SMILES: FC(F)(F)c1cc(N2CCCC3(C2)CN(c2cnc4cnn(Cc5ccccc5)c4n2)C3)ccn1]

Followed the general procedure A using 1-benzyl-6-chloro-1H-pyrazolo[3,4-b]pyrazine (20 mg, 82 μmol, 1.0 equiv.) and 6-[2-(trifluoromethyl)pyridin-4-yl]-2,6-diazaspiro[3.5]nonane hydrochloride (25.2 mg, 82 μmol, 1.0 equiv.) as the starting materials to give 2-{1-benzyl-1H-pyrazolo[3,4-b]pyrazin-6-yl}-6-[2-(trifluoromethyl)pyridin-4-yl]-2,6-diazaspiro[3.5]nonane (15 mg, 38%) as a colorless oil. ¹H NMR (500 MHz, DMSO-d₆) δ 8.58 (s, 1H), 8.24 (d, J=6.0 Hz, 1H), 7.97 (s, 1H), 7.37-7.25 (m, 6H), 7.13 (dd, J=6.1, 2.6 Hz, 1H), 5.47 (s, 2H), 3.92-3.77 (m, 4H), 3.65 (s, 2H), 3.38 (t, J=5.4 Hz, 5H), 1.88 (t, J=5.9 Hz, 2H), 1.64 (t, J=6.0 Hz, 2H). MS m/z: 480 [M+H]⁺.

### 2-{5-methyl-6-phenyl-5H-pyrrolo[2,3-b]pyrazine-7-carbonyl}-6-[2-(trifluoromethyl)pyridin-3-yl]-2,6-diazaspiro[3.5]nonane (105)

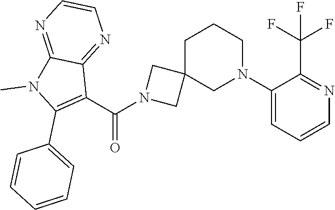 [SMILES: Cn1c(-c2ccccc2)c(C(=O)N2CC3(CCCN(c4cccnc4C(F)(F)F)C3)C2)c2nccnc21]

Followed the general procedure G using 5-methyl-6-phenyl-5H-pyrrolo[2,3-b]pyrazine-7-carboxylic acid (9.05 mg, 36 μmol, 1.0 equiv.) 6-[2-(trifluoromethyl)pyridin-3-yl]-2,6-diazaspiro[3.5]nonane hydrochloride (11 mg, 36 μmol, 1.0 equiv.) as the starting materials to give 2-{5-methyl-6-phenyl-5H-pyrrolo[2,3-b]pyrazine-7-carbonyl}-6-[2-(trifluoromethyl)pyridin-3-yl]-2,6-diazaspiro[3.5]nonane (11 mg, 61%) as a colorless oil. ¹H NMR (500 MHz, DMSO-d₆) δ 8.52-8.47 (m, 2H), 8.39 (d, J=2.6 Hz, 1H), 7.99 (dd, J=8.3, 1.4 Hz, 1H), 7.72 (dd, J=8.2, 4.5 Hz, 1H), 7.61-7.56 (m, 2H), 7.50-7.45 (m, 2H), 7.43-7.37 (m, 1H), 3.72 (s, 3H), 3.65 (s, 2H), 3.62-3.53 (m, 2H), 2.86 (d, J=10.9 Hz, 1H), 2.78 (d, J=10.9 Hz, 1H), 2.72-2.64 (m, 2H), 1.63-1.43 (m, 4H). MS m/z: 507 [M+H]⁺.

### 5-methyl-6-phenyl-N-{2-[2-(trifluoromethyl)pyridin-3-yl]-2-azaspiro[3.5]nonan-7-yl}-5H-pyrrolo[2,3-b]pyrazine-7-carboxamide (106)

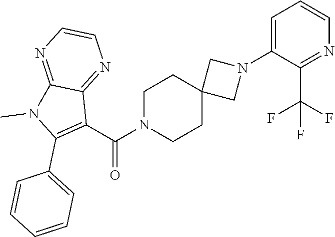 [SMILES: Cn1c(-c2ccccc2)c(C(=O)N2CCC3(CC2)CN(c2cccnc2C(F)(F)F)C3)c2nccnc21]

Step 1: tert-butyl 2-[2-(trifluoromethyl)pyridin-3-yl]-2,7-diazaspiro[3.5]nonane-7-carboxylate: Followed the general procedure D using tert-butyl 2,7-diazaspiro[3.5]nonane-7-carboxylate (50 mg, 221 μmol, 1.0 equiv) and 3-bromo-2-(trifluoromethyl)pyridine (50 mg, 221 μmol, 1.0 equiv.) as the starting materials to give tert-butyl 2-[2-(trifluoromethyl)pyridin-3-yl]-2,7-diazaspiro[3.5]nonane-7-carboxylate (70 mg, 85%) as a colorless oil. MS m/z: 372 [M+H]⁺.

Step 2: 2-[2-(trifluoromethyl)pyridin-3-yl]-2,7-diazaspiro[3.5]nonane hydrochloride: Followed the general procedure B using tert-butyl 2-[2-(trifluoromethyl)pyridin-3-yl]-2,7-diazaspiro[3.5]nonane-7-carboxylate (70 mg) as the starting material to give 2-[2-(trifluoromethyl)pyridin-3-yl]-2,7-diazaspiro[3.5]nonane hydrochloride (65 mg). MS m/z: 272 [M+H]⁺.

Step 3: 5-methyl-6-phenyl-N-{2-[2-(trifluoromethyl)pyridin-3-yl]-2-azaspiro[3.5]nonan-7-yl}-5H-pyrrolo[2,3-b]pyrazine-7-carboxamide: Followed the general procedure G using 5-methyl-6-phenyl-5H-pyrrolo[2,3-b]pyrazine-7-carboxylic acid (16.5 mg, 65 μmol, 1.0 equiv) and 2-[2-(trifluoromethyl)pyridin-3-yl]-2,7-diazaspiro[3.5]nonane hydrochloride (20 mg, 65 μmol, 1.0 equiv) as the starting materials to give 7-{5-methyl-6-phenyl-5H-pyrrolo[2,3-b]pyrazine-7-carbonyl}-2-[2-(trifluoromethyl)pyridin-3-yl]-2,7-diazaspiro[3.5]nonane (12 mg, 36%) as a colorless oil. ¹H NMR (500 MHz, DMSO) δ 8.50 (d, J=2.6 Hz, 1H), 8.39 (d, J=2.6 Hz, 1H), 7.97 (dd, J=4.3, 1.2 Hz, 1H), 7.63-7.53 (m, 5H), 7.45 (dd, J=8.5, 4.3 Hz, 1H), 7.04 (dd, J=8.7, 1.3 Hz, 1H), 3.77 (s, 3H), 3.76 (s, 2H), 3.70 (m, 2H), 3.59 (s, 2H), 3.32-3.17 (m, 2H), 1.70 (t, J=5.7 Hz, 2H), 1.43 (s, 2H). MS m/z: 507 [M+H]⁺.

### 5-methyl-6-phenyl-N-{2-[2-(trifluoromethyl)pyridin-3-yl]-2-azaspiro[3.5]nonan-7-yl}-5H-pyrrolo[2,3-b]pyrazine-7-carboxamide (107)

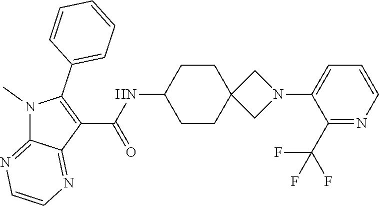 [SMILES: Cn1c(-c2ccccc2)c(C(=O)NC2CCC3(CC2)CN(c2cccnc2C(F)(F)F)C3)c2nccnc21]

Step 1: tert-butyl N-{2-[2-(trifluoromethyl)pyridin-3-yl]-2-azaspiro[3.5]nonan-7-yl}carbamate: Followed the general procedure D using tert-butyl 2,7-diazaspiro[3.5]nonane-7-carboxylate (53 mg, 221 μmol, 1.0 equiv) and 3-bromo-2-(trifluoromethyl)pyridine (50 mg, 221 μmol, 1.0 equiv.) as the starting materials to give tert-butyl N-{2-[2-(trifluoromethyl)pyridin-3-yl]-2-azaspiro[3.5]nonan-7-yl}carbamate (57 mg, 67%) as a colorless oil. MS m/z: 386 [M+H]⁺.

Step 2: 2-[2-(trifluoromethyl)pyridin-3-yl]-2-azaspiro[3.5]nonan-7-amine hydrochloride: Followed the general procedure B using tert-butyl N-{2-[2-(trifluoromethyl)pyridin-3-yl]-2-azaspiro[3.5]nonan-7-yl}carbamate (57 mg) as the starting material to give 2-[2-(trifluoromethyl)pyridin-3-yl]-2-azaspiro[3.5]nonan-7-amine hydrochloride (50 mg). MS m/z: 286 [M+H]⁺.

Step 3: 5-methyl-6-phenyl-N-{2-[2-(trifluoromethyl)pyridin-3-yl]-2-azaspiro[3.5]nonan-7-yl}-5H-pyrrolo[2,3-b]pyrazine-7-carboxamide: Followed the general procedure G using 5-methyl-6-phenyl-5H-pyrrolo[2,3-b]pyrazine-7-carboxylic acid (15.7 mg, 62 μmol, 1.0 equiv) and 2-[2-(trifluoromethyl)pyridin-3-yl]-2-azaspiro[3.5]nonan-7-amine hydrochloride (20 mg, 62.2 μmol, 1.0 equiv) as the starting materials to give 5-methyl-6-phenyl-N-{2-[2-(trifluoromethyl)pyridin-3-yl]-2-azaspiro[3.5]nonan-7-yl}-5H-pyrrolo[2,3-b]pyrazine-7-carboxamide (28 mg, 87%) as a colorless oil. ¹H NMR (500 MHz, DMSO-d₆) δ 8.63 (d, J=2.6 Hz, 1H), 8.52 (d, J=7.8 Hz, 1H), 8.49 (d, J=2.6 Hz, 1H), 7.92 (dd, J=4.3, 1.2 Hz, 1H), 7.58-7.51 (m, 5H), 7.46 (t, J=4.0 Hz, 1H), 7.08 (dd, J=8.7, 1.3 Hz, 1H), 5.12 (t, J=6.1 Hz, 1H), 3.72 (s, 2H), 3.64 (s, 2H), 3.61 (s, 3H), 1.96-1.87 (m, 1H), 1.80-1.71 (m, 2H), 1.70-1.54 (m, 3H), 1.48-1.33 (m, 2H). MS m/z: 521 [M+H]⁺.

### 6-{5-methyl-6-phenyl-5H-pyrrolo[2,3-b]pyrazine-7-carbonyl}-2-{[2-(trifluoromethyl)pyridin-3-yl]oxy}-6-azaspiro[3.5]nonane (108)

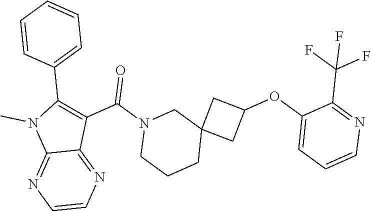 [SMILES: Cn1c(-c2ccccc2)c(C(=O)N2CCCC3(CC(Oc4cccnc4C(F)(F)F)C3)C2)c2nccnc21]

Followed the general procedure G using 5-methyl-6-phenyl-5H-pyrrolo[2,3-b]pyrazine-7-carboxylic acid (20 mg, 79 μmol, 1.0 equiv) and 2-{[2-(trifluoromethyl)pyridin-3-yl]oxy}-6-azaspiro[3.5]nonane hydrochloride (25.5 mg, 79 μmol, 1.0 equiv) as the starting materials to give 6-{5-methyl-6-phenyl-5H-pyrrolo[2,3-b]pyrazine-7-carbonyl}-2-{[2-(trifluoromethyl)pyridin-3-yl]oxy}-6-azaspiro[3.5]nonane (5.2 mg, 13%) as a colorless oil. MS m/z: 522 [M+H]⁺.

### Example 109: N-[1-(2,2-difluoroethyl)-1H-pyrazolo[3,4-b]pyrazin-6-yl]-2-[2-(trifluoromethyl)pyridin-3-yl]-2-azaspiro[3.5]nonan-7-amine

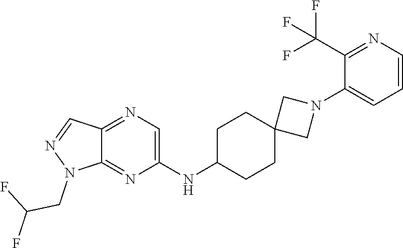 [SMILES: FC(F)Cn1ncc2ncc(NC3CCC4(CC3)CN(c3cccnc3C(F)(F)F)C4)nc21]

Followed the General Procedure A to afford the desired product as a colorless oil (7 mg, 12%). ¹H NMR (500 MHz, DMSO-d₆) δ 8.03 (s, 1H), 7.98 (dd, J=4.3, 1.2 Hz, 1H), 7.93 (s, 1H), 7.71 (d, J=7.2 Hz, 1H), 7.46 (dd, J=8.5, 4.3 Hz, 1H), 7.10 (dd, J=8.6, 1.3 Hz, 1H), 6.43 (tt, J=54.9, 3.9 Hz, 1H), 4.64 (td, J=15.0, 3.9 Hz, 2H), 3.83 (d, J=7.0 Hz, 1H), 3.80 (s, 2H), 3.74 (s, 2H), 1.96 (td, J=11.9, 10.4, 4.6 Hz, 4H), 1.73-1.61 (m, 2H), 1.43-1.32 (m, 2H). MS m/z: 468 [M+H]⁺.

### Example 110: 2-[1-(2,2-difluoroethyl)-1H-pyrazolo[3,4-b]pyrazin-6-yl]-8-[6-(trifluoromethyl)pyridin-3-yl]-2,8-diazaspiro[4.5]decan-3-one

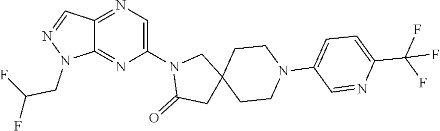 [SMILES: O=C1CC2(CCN(c3ccc(C(F)(F)F)nc3)CC2)CN1c1cnc2cnn(CC(F)F)c2n1]

Followed the General Procedure A to afford the desired product as a white solid (10 mg, 39%). ¹H NMR (500 MHz, DMSO-d₆) δ 9.65 (s, 1H), 8.49 (s, 1H), 8.47 (d, J=3.0 Hz, 1H), 7.64 (d, J=8.9 Hz, 1H), 7.48 (dd, J=8.9, 2.9 Hz, 1H), 6.52 (tt, J=54.7, 3.7 Hz, 1H), 4.87 (td, J=15.1, 3.7 Hz, 2H), 3.97 (s, 2H), 3.61 (dt, J=13.4, 5.1 Hz, 2H), 3.37 (dd, J=4.7, 3.2 Hz, 2H), 2.75 (s, 2H), 1.85-1.72 (m, 4H). MS m/z: 482 [M+H]⁺.

### Example 111: 2-[1-(2,2-difluoroethyl)-1H-pyrazolo[3,4-b]pyrazin-6-yl]-8-[5-(trifluoromethyl)pyridin-3-yl]-2,8-diazaspiro[4.5]decan-3-one

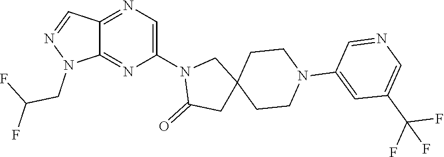 [SMILES: O=C1CC2(CCN(c3cncc(C(F)(F)F)c3)CC2)CN1c1cnc2cnn(CC(F)F)c2n1]

Followed the General Procedure A to afford the desired product as a white solid (5 mg, 19%). MS m/z: 482 [M+H]⁺.

### Example 112: 2-[1-(2,2-difluoroethyl)-1H-pyrazolo[3,4-b]pyrazin-6-yl]-8-[5-(trifluoromethyl)pyridin-2-yl]-2,8-diazaspiro[4.5]decan-3-one

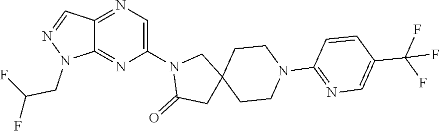 [SMILES: O=C1CC2(CCN(c3ccc(C(F)(F)F)cn3)CC2)CN1c1cnc2cnn(CC(F)F)c2n1]

Followed the General Procedure A to afford the desired product as a white solid (13 mg, 50%). ¹H NMR (500 MHz, DMSO-d₆) δ 9.65 (s, 1H), 8.49 (s, 1H), 8.42 (dd, J=1.7, 0.9 Hz, 1H), 7.79 (dd, J=9.1, 2.6 Hz, 1H), 7.02 (d, J=9.1 Hz, 1H), 6.66-6.39 (m, 1H), 4.88 (td, J=15.1, 3.7 Hz, 2H), 3.98 (s, 2H), 3.93 (dt, J=13.6, 5.0 Hz, 2H), 3.57 (dt, J=13.0, 6.0 Hz, 2H), 2.76 (s, 2H), 1.73 (t, J=5.6 Hz, 4H). MS m/z: 482 [M+H]⁺.

### Example 113: 2-[1-(2,2-difluoroethyl)-1H-pyrazolo[3,4-b]pyrazin-6-yl]-8-[3-(trifluoromethyl)pyridin-2-yl]-2,8-diazaspiro[4.5]decan-3-one

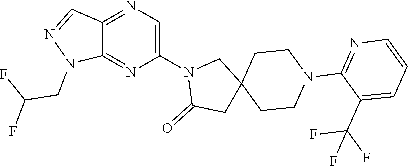

Followed the General Procedure A to afford the desired product as a white solid (10 mg, 39%). ¹H NMR (500 MHz, DMSO-d₆) δ 9.65 (s, 1H), 8.53 (dd, J=4.8, 1.8 Hz, 1H), 8.49 (s, 1H), 8.07 (dd, J=7.8, 1.9 Hz, 1H), 7.19 (dd, J=7.9, 4.6 Hz, 1H), 6.53 (tt, J=54.6, 3.7 Hz, 1H), 4.90 (td, J=15.1, 3.7 Hz, 2H), 4.00 (s, 2H), 3.32-3.24 (m, 2H), 3.19 (ddd, J=12.5, 7.8, 3.6 Hz, 2H), 2.73 (s, 2H), 1.83 (dq, J=16.4, 7.2, 5.5 Hz, 4H). MS m/z: 482 [M+H]⁺.

### Example 114: 2-[1-(2,2-difluoroethyl)-1H-pyrazolo[3,4-b]pyrazin-6-yl]-6-[6-(trifluoromethyl)pyridin-2-yl]-2,6-diazaspiro[3.5]nonan-7-one

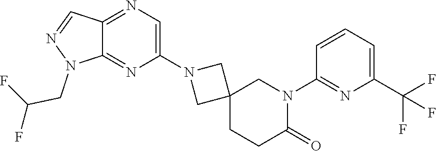 [SMILES: O=C1CCC2(CN(c3cnc4cnn(CC(F)F)c4n3)C2)CN1c1cccc(C(F)(F)F)n1]

Followed the General Procedure D to afford the desired product as a colorless oil (28 mg, 81%). ¹H NMR (500 MHz, DMSO-d₆) δ 8.15 (s, 1H), 8.10-8.01 (m, 2H), 7.93 (s, 1H), 7.71 (dd, J=7.4, 1.0 Hz, 1H), 6.43 (tt, J=54.9, 3.8 Hz, 1H), 4.66 (td, J=14.9, 3.9 Hz, 2H), 4.13-4.11 (m, 6H), 2.65 (t, J=6.8 Hz, 2H), 2.20 (t, J=6.9 Hz, 2H). MS m/z: 468 [M+H]⁺.

### Example 115: 2-[1-(2,2-difluoroethyl)-1H-pyrazolo[3,4-b]pyrazin-6-yl]-6-[6-(trifluoromethyl)pyridin-2-yl]-2,6-diazaspiro[3.5]nonan-5-one

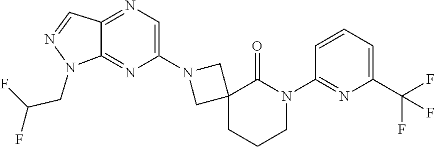

Followed the General Procedure D to afford the desired product as an off-white solid (29 mg, 84%). ¹H NMR (500 MHz, DMSO-d₆) δ 8.17 (s, 1H), 8.12-8.02 (m, 2H), 7.99 (s, 1H), 7.73 (dd, J=7.2, 1.1 Hz, 1H), 6.45 (tt, J=54.9, 3.8 Hz, 1H), 4.69 (td, J=15.0, 3.8 Hz, 2H), 4.50 (d, J=8.5 Hz, 2H), 4.04 (dd, J=13.5, 7.7 Hz, 2H), 3.89 (t, J=6.0 Hz, 2H), 2.34-2.28 (m, 2H), 1.95 (dd, J=7.7, 4.2 Hz, 2H). MS m/z: 468 [M+H]⁺.

### Example 116: 8-[1-(2,2-difluoroethyl)-1H-pyrazolo[3,4-b]pyrazin-6-yl]-2-(2-methoxyethyl)-2,8-diazaspiro[4.5]decan-1-one

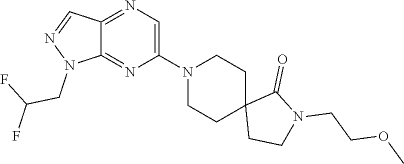 [SMILES: COCCN1CCC2(CCN(c3cnc4cnn(CC(F)F)c4n3)CC2)C1=O]

Followed the General Procedure E to afford the desired product as a colorless oil (17 mg, 58%). ¹H NMR (500 MHz, DMSO-d₆) δ 8.48 (s, 1H), 8.14 (s, 1H), 6.44 (tt, J=54.9, 3.8 Hz, 1H), 4.69 (td, J=15.0, 3.8 Hz, 2H), 4.39 (dt, J=13.6, 4.2 Hz, 2H), 3.43 (t, J=5.5 Hz, 2H), 3.39-3.34 (m, 4H), 3.28 (ddd, J=14.0, 11.5, 3.0 Hz, 2H), 3.24 (s, 3H), 2.01 (t, J=6.9 Hz, 2H), 1.70 (ddd, J=13.4, 11.5, 4.2 Hz, 2H), 1.51-1.41 (m, 2H). MS m/z: 395 [M+H]⁺.

### Example 117: 2-[1-(2,2-difluoroethyl)-1H-pyrazolo[3,4-b]pyrazin-6-yl]-7-[2-(trifluoromethyl)pyridin-4-yl]-2,7-diazaspiro[3.5]nonane

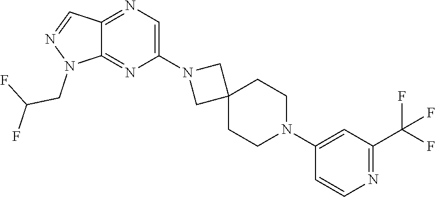 [SMILES: FC(F)Cn1ncc2ncc(N3CC4(CCN(c5ccnc(C(F)(F)F)c5)CC4)C3)nc21]

Followed the General Procedure D to afford the desired product as a white solid (17 mg, 43%). ¹H NMR (500 MHz, DMSO-d₆) δ 8.25 (d, J=6.0 Hz, 1H), 8.14 (s, 1H), 7.92 (s, 1H), 7.25 (d, J=2.6 Hz, 1H), 7.09 (dd, J=6.1, 2.6 Hz, 1H), 6.44 (tt, J=54.8, 3.7 Hz, 1H), 4.66 (td, J=15.0, 3.8 Hz, 2H), 3.98 (s, 4H), 3.51 (dd, J=7.0, 4.3 Hz, 4H), 1.85 (t, J=5.6 Hz, 4H). MS m/z: 454 [M+H]⁺.

### Example 118: 2-[1-(2,2-difluoroethyl)-1H-pyrazolo[3,4-b]pyrazin-6-yl]-8-[2-(trifluoromethyl)pyridin-4-yl]-2,8-diazaspiro[4.5]decan-1-one

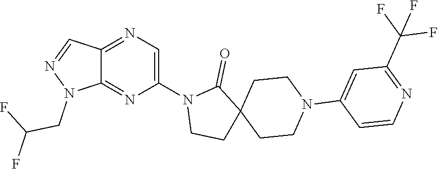 [SMILES: O=C1N(c2cnc3cnn(CC(F)F)c3n2)CCC12CCN(c1ccnc(C(F)(F)F)c1)CC2]

Followed the General Procedure D to afford the desired product as an off-white solid (10 mg, 31%). ¹H NMR (500 MHz, DMSO-d₆) δ 9.66 (s, 1H), 8.50 (s, 1H), 8.28 (d, J=6.0 Hz, 1H), 7.27 (d, J=2.6 Hz, 1H), 7.10 (dd, J=6.0, 2.6 Hz, 1H), 6.52 (tt, J=54.6, 3.6 Hz, 1H), 4.88 (td, J=15.1, 3.6 Hz, 2H), 4.14-4.07 (m, 2H), 4.02 (dd, J=13.7, 3.9 Hz, 2H), 3.26-3.18 (m, 2H), 2.25 (t, J=7.1 Hz, 2H), 1.83 (td, J=12.5, 11.3, 4.1 Hz, 2H), 1.72 (d, J=13.5 Hz, 2H). MS m/z: 482 [M+H]⁺.

### Example 119: 2-[1-(2,2-difluoroethyl)-1H-pyrazolo[3,4-b]pyrazin-6-yl]-8-[6-(trifluoromethyl)pyridin-3-yl]-2,8-diazaspiro[4.5]decan-1-one

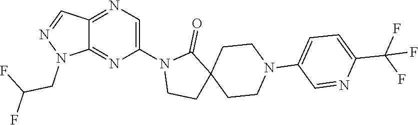 [SMILES: O=C1N(c2cnc3cnn(CC(F)F)c3n2)CCC12CCN(c1ccc(C(F)(F)F)nc1)CC2]

Followed the General Procedure A to afford the desired product as a white solid (19 mg, 59%). ¹H NMR (500 MHz, DMSO-d₆) δ 9.67 (s, 1H), 8.50 (s, 1H), 8.48 (d, J=2.9 Hz, 1H), 7.65 (d, J=8.8 Hz, 1H), 7.50 (dd, J=8.9, 2.9 Hz, 1H), 6.52 (tt, J=54.6, 3.6 Hz, 1H), 4.88 (td, J=15.2, 3.6 Hz, 2H), 4.10 (dd, J=7.8, 6.3 Hz, 2H), 3.93 (dt, J=13.3, 4.1 Hz, 2H), 3.15 (ddd, J=13.5, 11.4, 2.9 Hz, 2H), 2.25 (t, J=7.1 Hz, 2H), 1.89 (ddd, J=13.3, 11.4, 4.2 Hz, 2H), 1.74 (d, J=13.0 Hz, 2H). MS m/z: 482 [M+H]⁺.

### Example 120: 2-[1-(2,2-difluoroethyl)-1H-pyrazolo[3,4-b]pyrazin-6-yl]-8-[3-(trifluoromethyl)pyridin-2-yl]-2,8-diazaspiro[4.5]decan-1-one

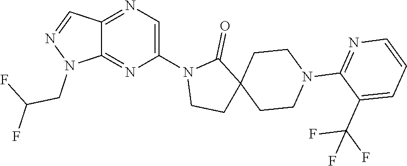 [SMILES: O=C1N(c2cnc3cnn(CC(F)F)c3n2)CCC12CCN(c1ncccc1C(F)(F)F)CC2]

Followed the General Procedure A to afford the desired product as a white solid (17 mg, 53%). ¹H NMR (500 MHz, DMSO-d₆) δ 9.69 (s, 1H), 8.54 (dd, J=4.3, 1.5 Hz, 1H), 8.50 (s, 1H), 8.08 (dd, J=7.8, 1.9 Hz, 1H), 7.20 (ddd, J=7.8, 4.8, 0.9 Hz, 1H), 6.52 (tt, J=54.6, 3.6 Hz, 1H), 4.88 (td, J=15.1, 3.6 Hz, 2H), 4.09 (dd, J=7.8, 6.4 Hz, 2H), 3.50 (dd, J=13.1, 3.9 Hz, 2H), 3.09 (td, J=12.6, 2.5 Hz, 2H), 2.27-2.18 (m, 2H), 1.96 (td, J=12.5, 3.9 Hz, 2H), 1.73 (d, J=14.1 Hz, 2H). MS m/z: 482 [M+H]⁺.

### Example 121: 2-[1-(2,2-difluoroethyl)-1H-pyrazolo[3,4-b]pyrazin-6-yl]-8-[5-(trifluoromethyl)pyridin-2-yl]-2,8-diazaspiro[4.5]decan-1-one

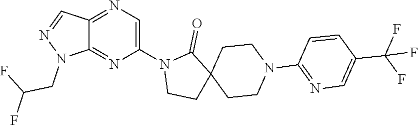 [SMILES: O=C1N(c2cnc3cnn(CC(F)F)c3n2)CCC12CCN(c1ccc(C(F)(F)F)cn1)CC2]

Followed the General Procedure A to afford the desired product as a white solid (25 mg, 77%). ¹H NMR (500 MHz, DMSO-d₆) δ 9.67 (s, 1H), 8.50 (s, 1H), 8.43 (s, 1H), 7.81 (dd, J=9.1, 2.7 Hz, 1H), 7.04 (d, J=9.1 Hz, 1H), 6.68-6.38 (m, 1H), 4.89 (td, J=15.1, 3.6 Hz, 2H), 4.34 (dt, J=13.9, 4.0 Hz, 2H), 4.11 (t, J=7.0 Hz, 2H), 3.33-3.23 (m, 2H), 2.28 (t, J=7.1 Hz, 2H), 1.84-1.75 (m, 2H), 1.75-1.67 (m, 2H). MS m/z: 482 [M+H]⁺.

### Example 122: 2-[1-(2,2-difluoroethyl)-1H-pyrazolo[3,4-b]pyrazin-6-yl]-8-[5-(trifluoromethyl)pyridin-3-yl]-2,8-diazaspiro[4.5]decan-1-one

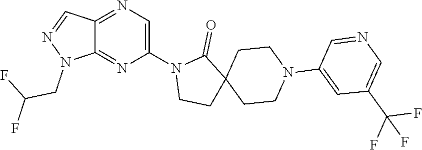 [SMILES: O=C1N(c2cnc3cnn(CC(F)F)c3n2)CCC12CCN(c1cncc(C(F)(F)F)c1)CC2]

Followed the General Procedure A to afford the desired product as a white solid (7 mg, 22%). ¹H NMR (500 MHz, DMSO-d₆) δ 9.69 (s, 1H), 8.65 (d, J=2.8 Hz, 1H), 8.51 (s, 1H), 8.29 (s, 1H), 7.64 (s, 1H), 6.66-6.40 (m, 1H), 4.89 (td, J=15.1, 3.6 Hz, 2H), 4.15-4.07 (m, 2H), 3.96-3.86 (m, 2H), 3.15-3.04 (m, 2H), 2.24 (t, J=7.1 Hz, 2H), 1.96-1.86 (m, 2H), 1.80-1.70 (m, 2H). MS m/z: 482 [M+H]⁺.

### Example 123: 2-[1-(2,2-difluoroethyl)-1H-pyrazolo[3,4-b]pyrazin-6-yl]-7-[3-(trifluoromethyl)pyridin-2-yl]-2,7-diazaspiro[3.5]nonane

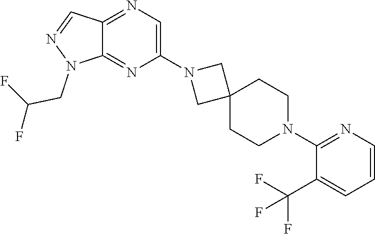 [SMILES: FC(F)Cn1ncc2ncc(N3CC4(CCN(c5ncccc5C(F)(F)F)CC4)C3)nc21]

Followed the General Procedure A to afford the desired product as a colorless oil (23 mg, 74%). ¹H NMR (500 MHz, DMSO-d₆) δ 8.53 (dd, J=4.3, 1.5 Hz, 1H), 8.15 (s, 1H), 8.07 (dd, J=7.8, 1.8 Hz, 1H), 7.95 (s, 1H), 7.25-7.15 (m, 1H), 6.59-6.31 (m, 1H), 4.68 (td, J=15.0, 3.8 Hz, 2H), 3.98 (s, 4H), 3.22-3.13 (m, 4H), 1.97-1.87 (m, 4H). MS m/z: 454 [M+H]⁺.

### Example 124: 2-[1-(2,2-difluoroethyl)-1H-pyrazolo[3,4-b]pyrazin-6-yl]-7-[5-(trifluoromethyl)pyridin-2-yl]-2,7-diazaspiro[3.5]nonane

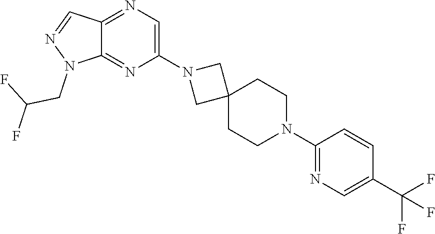 [SMILES: FC(F)Cn1ncc2ncc(N3CC4(CCN(c5ccc(C(F)(F)F)cn5)CC4)C3)nc21]

Followed the General Procedure A to afford the desired product as a white solid (22 mg, 71%). ¹H NMR (500 MHz, DMSO-d₆) δ 8.40 (dq, J=2.8, 0.9 Hz, 1H), 8.14 (s, 1H), 7.93 (s, 1H), 7.81-7.72 (m, 1H), 7.02 (d, J=9.1 Hz, 1H), 6.44 (tt, J=54.8, 3.8 Hz, 1H), 4.67 (td, J=15.0, 3.8 Hz, 2H), 3.98 (s, 4H), 3.69 (dd, J=7.3, 3.5 Hz, 4H), 1.83 (t, J=5.5 Hz, 4H). MS m/z: 454 [M+H]⁺.

### Example 125: 2-[1-(2,2-difluoroethyl)-1H-pyrazolo[3,4-b]pyrazin-6-yl]-7-[6-(trifluoromethyl)pyridin-2-yl]-2,7-diazaspiro[3.5]nonane

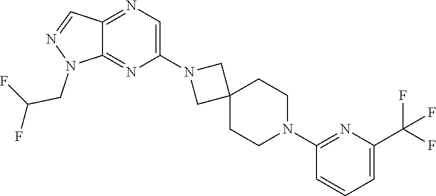 [SMILES: FC(F)Cn1ncc2ncc(N3CC4(CCN(c5cccc(C(F)(F)F)n5)CC4)C3)nc21]

Followed the General Procedure A to afford the desired product as a white solid (25 mg, 80%). ¹H NMR (500 MHz, DMSO-d₆) δ 8.15 (s, 1H), 7.94 (s, 1H), 7.77-7.70 (m, 1H), 7.19 (d, J=8.8 Hz, 1H), 7.01 (d, J=7.2 Hz, 1H), 6.58-6.30 (m, 1H), 4.67 (td, J=15.0, 3.8 Hz, 2H), 3.98 (s, 4H), 3.66-3.60 (m, 4H), 1.84 (t, J=5.5 Hz, 4H). MS m/z: 454 [M+H]⁺.

### Example 126: 2-[1-(2,2-difluoroethyl)-1H-pyrazolo[3,4-b]pyrazin-6-yl]-7-[5-(trifluoromethyl)pyridin-3-yl]-2,7-diazaspiro[3.5]nonane

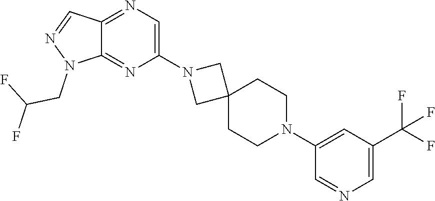 [SMILES: FC(F)Cn1ncc2ncc(N3CC4(CCN(c5cncc(C(F)(F)F)c5)CC4)C3)nc21]

Followed the General Procedure A to afford the desired product as a white solid (25 mg, 80%). ¹H NMR (500 MHz, DMSO-d₆) δ 8.62 (d, J=2.8 Hz, 1H), 8.26 (dd, J=1.9, 0.9 Hz, 1H), 8.14 (s, 1H), 7.93 (s, 1H), 7.60 (t, J=2.5 Hz, 1H), 6.44 (tt, J=54.9, 3.8 Hz, 1H), 4.67 (td, J=15.0, 3.8 Hz, 2H), 3.97 (s, 4H), 3.44-3.36 (m, 4H), 1.97-1.83 (m, 4H). MS m/z: 454 [M+H]⁺.

### Example 127: 2-[1-(2,2-difluoroethyl)-1H-pyrazolo[3,4-b]pyrazin-6-yl]-7-[6-(trifluoromethyl)pyridin-3-yl]-2,7-diazaspiro[3.5]nonane

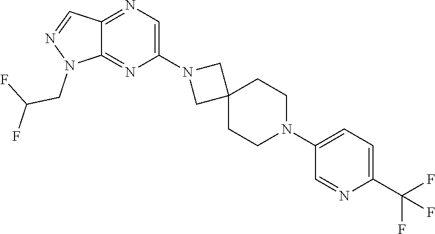 [SMILES: FC(F)Cn1ncc2ncc(N3CC4(CCN(c5ccc(C(F)(F)F)nc5)CC4)C3)nc21]

Followed the General Procedure A to afford the desired product as a white solid (27 mg, 81%). ¹H NMR (500 MHz, DMSO-d₆) δ 8.46 (d, J=2.9 Hz, 1H), 8.14 (s, 1H), 7.93 (s, 1H), 7.63 (d, J=8.8 Hz, 1H), 7.47 (dd, J=8.9, 2.9 Hz, 1H), 6.44 (tt, J=54.9, 3.8 Hz, 1H), 4.67 (td, J=15.0, 3.8 Hz, 2H), 3.98 (s, 4H), 3.48-3.38 (m, 4H), 1.93-1.81 (m, 4H). MS m/z: 454 [M+H]⁺.

### Example 128: 2-[1-(2,2-difluoroethyl)-1H-pyrazolo[3,4-b]pyrazin-6-yl]-7-[4-(trifluoromethyl)pyridin-3-yl]-2,7-diazaspiro[3.5]nonane

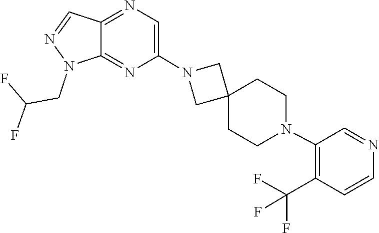 [SMILES: FC(F)Cn1ncc2ncc(N3CC4(CCN(c5cnccc5C(F)(F)F)CC4)C3)nc21]

Followed the General Procedure A to afford the desired product as a white solid (23 mg, 74%). ¹H NMR (500 MHz, DMSO-d₆) δ 8.83 (s, 1H), 8.57 (d, J=5.0 Hz, 1H), 8.15 (s, 1H), 7.95 (s, 1H), 7.67 (d, J=5.0 Hz, 1H), 6.62-6.31 (m, 1H), 4.68 (td, J=15.0, 3.8 Hz, 2H), 4.00 (s, 4H), 3.00 (t, J=5.3 Hz, 4H), 1.94 (t, J=5.3 Hz, 4H). MS m/z: 454 [M+H]⁺.

### Example 129: 2-[1-(2,2-difluoroethyl)-1H-pyrazolo[3,4-b]pyrazin-6-yl]-7-[2-(trifluoromethyl)pyridin-3-yl]-2,7-diazaspiro[3.5]nonane

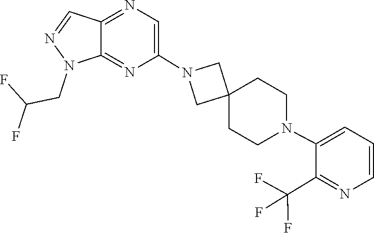 [SMILES: FC(F)Cn1ncc2ncc(N3CC4(CCN(c5cccnc5C(F)(F)F)CC4)C3)nc21]

Followed the General Procedure A to afford the desired product as a white solid (22 mg, 71%). ¹H NMR (500 MHz, DMSO-d₆) δ 8.45 (dd, J=4.5, 1.4 Hz, 1H), 8.14 (s, 1H), 8.04-7.98 (m, 1H), 7.94 (s, 1H), 7.70 (dd, J=8.2, 4.5 Hz, 1H), 6.58-6.31 (m, 1H), 4.67 (td, J=15.0, 3.8 Hz, 2H), 3.98 (s, 4H), 2.90 (t, J=5.3 Hz, 4H), 1.94 (t, J=5.3 Hz, 4H). MS m/z: 454 [M+H]⁺.

### Example 130: N-[1-(2,2-difluoroethyl)-1H-pyrazolo[3,4-b]pyrazin-6-yl]-2-[6-(trifluoromethyl)pyridin-2-yl]-2-azaspiro[3.5]nonan-7-amine

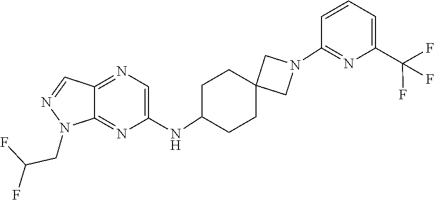 [SMILES: FC(F)Cn1ncc2ncc(NC3CCC4(CC3)CN(c3cccc(C(F)(F)F)n3)C4)nc21]

Followed the General Procedure A to afford the desired product as a colorless oil (1.8 mg, 4%). MS m/z: 469 [M+H]⁺.

### Example 131: 2-[1-(2,2-difluoroethyl)-1H-pyrazolo[3,4-b]pyrazin-6-yl]-6-({[2-(trifluoromethyl)pyridin-3-yl]oxy}methyl)-2-azaspiro[3.3]heptane

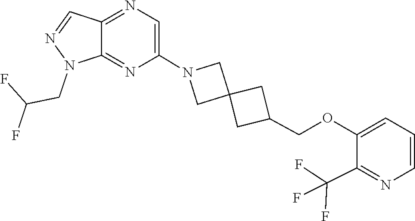 [SMILES: FC(F)Cn1ncc2ncc(N3CC4(CC(COc5cccnc5C(F)(F)F)C4)C3)nc21]

Followed the General Procedure A to afford the desired product as a white solid (10 mg, 24%). ¹H NMR (500 MHz, DMSO-d₆) δ 8.26 (dd, J=4.6, 1.1 Hz, 1H), 8.13 (s, 1H), 7.89 (s, 1H), 7.80 (d, J=8.0 Hz, 1H), 7.70 (dd, J=8.6, 4.5 Hz, 1H), 6.43 (tt, J=54.9, 3.8 Hz, 1H), 4.66 (td, J=15.0, 3.8 Hz, 2H), 4.25 (s, 2H), 4.14 (d, J=6.0 Hz, 2H), 4.12 (s, 2H), 2.69 (dq, J=8.2, 6.2 Hz, 1H), 2.43-2.33 (m, 2H), 2.23-2.14 (m, 2H). MS m/z: 455 [M+H]⁺.

### Example 132: 2-[1-(2,2-difluoroethyl)-1H-pyrazolo[3,4-b]pyrazin-6-yl]-6-({[4-(trifluoromethyl)pyridin-2-yl]oxy}methyl)-2-azaspiro[3.3]heptane

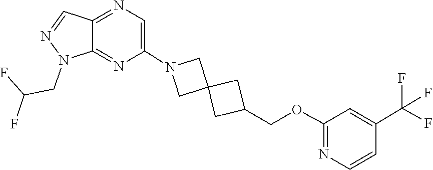 [SMILES: FC(F)Cn1ncc2ncc(N3CC4(CC(COc5cc(C(F)(F)F)ccn5)C4)C3)nc21]

Followed the General Procedure A to afford the desired product as a colorless oil (5 mg, 12%). MS m/z: 455 [M+H]⁺.

### Example 133: 2-[1-(2,2-difluoroethyl)-1H-pyrazolo[3,4-b]pyrazin-6-yl]-6-({[6-(trifluoromethyl)pyridin-3-yl]oxy}methyl)-2-azaspiro[3.3]heptane

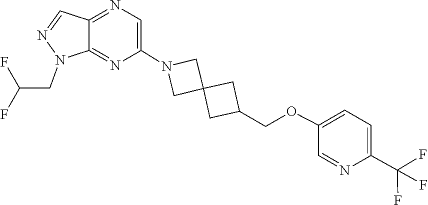 [SMILES: FC(F)Cn1ncc2ncc(N3CC4(CC(COc5ccc(C(F)(F)F)nc5)C4)C3)nc21]

Followed the General Procedure A to afford the desired product as a colorless oil (8 mg, 19%). ¹H NMR (500 MHz, DMSO-d₆) δ 8.50 (d, J=2.8 Hz, 1H), 8.16 (s, 1H), 7.94 (s, 1H), 7.88 (d, J=8.7 Hz, 1H), 7.65 (dd, J=8.7, 2.8 Hz, 1H), 6.58-6.33 (m, 1H), 4.69 (td, J=15.0, 3.8 Hz, 2H), 4.25 (s, 2H), 4.21 (s, 2H), 4.17 (d, J=6.4 Hz, 2H), 2.75-2.64 (m, 1H), 2.50-2.42 (m, 2H), 2.22-2.14 (m, 2H). MS m/z: 455 [M+H]⁺.

### Example 134: 2-[1-(2,2-difluoroethyl)-1H-pyrazolo[3,4-b]pyrazin-6-yl]-6-({[6-(trifluoromethyl)pyridin-2-yl]oxy}methyl)-2-azaspiro[3.3]heptane

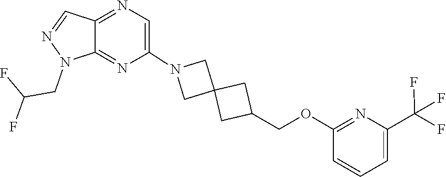 [SMILES: FC(F)Cn1ncc2ncc(N3CC4(CC(COc5cccc(C(F)(F)F)n5)C4)C3)nc21]

Followed the General Procedure A to afford the desired product as a colorless oil (15 mg, 36%). ¹H NMR (500 MHz, DMSO-d₆) δ 8.13 (s, 1H), 7.97 (ddd, J=8.3, 7.4, 0.8 Hz, 1H), 7.90 (s, 1H), 7.48 (d, J=7.3 Hz, 1H), 7.17 (dd, J=8.4, 0.9 Hz, 1H), 6.43 (tt, J=54.9, 3.8 Hz, 1H), 4.66 (td, J=15.0, 3.8 Hz, 2H), 4.28 (d, J=6.6 Hz, 2H), 4.22 (s, 2H), 4.16 (s, 2H), 2.69-2.61 (m, 1H), 2.45-2.38 (m, 2H), 2.19-2.11 (m, 2H). MS m/z: 455 [M+H]⁺.

### Example 135: 8-[1-(2,2-difluoroethyl)-1H-pyrazolo[3,4-b]pyrazin-6-yl]-2-[2-(trifluoromethyl)pyridin-3-yl]-2,8-diazaspiro[4.5]decan-1-one

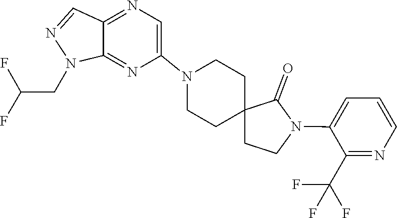

Followed the General Procedure E to afford the desired product as a colorless oil (7 mg, 20%). ¹H NMR (500 MHz, DMSO-d₆) δ 8.75 (dd, J=4.5, 1.4 Hz, 1H), 8.52 (s, 1H), 8.15 (s, 1H), 8.10 (dd, J=8.2, 1.5 Hz, 1H), 7.87 (dd, J=8.1, 4.6 Hz, 1H), 6.45 (tt, J=54.9, 3.8 Hz, 1H), 4.71 (td, J=15.0, 3.8 Hz, 2H), 4.41 (dt, J=13.6, 4.3 Hz, 2H), 3.74 (t, J=6.8 Hz, 2H), 3.44-3.38 (m, 2H), 2.28 (t, J=6.8 Hz, 2H), 1.80 (ddd, J=13.3, 11.1, 4.2 Hz, 2H), 1.68 (dt, J=13.4, 3.5 Hz, 2H). MS m/z: 482 [M+H]⁺.

### Example 136: 2-[1-(2,2,2-trifluoroethyl)-1H-pyrazolo[3,4-b]pyrazin-6-yl]-6-[6-(trifluoromethyl)pyridin-3-yl]-2,6-diazaspiro[3.5]nonane

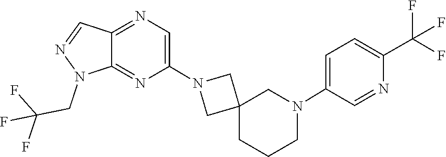 [SMILES: FC(F)(F)Cn1ncc2ncc(N3CC4(CCCN(c5ccc(C(F)(F)F)nc5)C4)C3)nc21]

Followed the General Procedure A to afford the desired product as a white solid (39 mg, 85%). ¹H NMR (500 MHz, DMSO-d₆) δ 8.50 (d, J=2.9 Hz, 1H), 8.19 (s, 1H), 7.97 (s, 1H), 7.62 (d, J=8.8 Hz, 1H), 7.53 (dd, J=8.9, 2.9 Hz, 1H), 5.11 (q, J=9.1 Hz, 2H), 3.93 (d, J=27.0 Hz, 4H), 3.57 (s, 2H), 3.32-3.24 (m, 2H), 1.89 (dd, J=12.7, 6.9 Hz, 2H), 1.69 (s, 2H). MS m/z: 473 [M+H]⁺.

### Example 137: 2-[1-(2,2-difluoroethyl)-3-methyl-1H-pyrazolo[3,4-b]pyrazin-6-yl]-6-[6-(trifluoromethyl)pyridin-3-yl]-2,6-diazaspiro[3.5]nonane

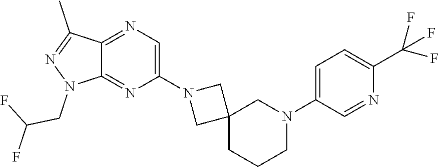 [SMILES: Cc1nn(CC(F)F)c2nc(N3CC4(CCCN(c5ccc(C(F)(F)F)nc5)C4)C3)cnc12]

Followed the General Procedure A to afford the desired product as a white solid (29 mg, 64%). ¹H NMR (500 MHz, DMSO-d₆) δ 8.50 (d, J=2.8 Hz, 1H), 7.86 (s, 1H), 7.62 (d, J=8.8 Hz, 1H), 7.53 (dd, J=8.9, 2.9 Hz, 1H), 6.40 (tt, J=55.0, 3.9 Hz, 1H), 4.57 (td, J=14.9, 3.9 Hz, 2H), 4.00-3.83 (m, 4H), 3.56 (s, 2H), 3.32-3.23 (m, 2H), 2.41 (s, 3H), 1.87 (t, J=5.9 Hz, 2H), 1.70 (q, J=5.8 Hz, 2H). MS m/z: 469 [M+H]⁺.

### Example 138: 2-[6-(1,3,4-thiadiazol-2-yl)pyrazin-2-yl]-6-[6-(trifluoromethyl)pyridin-3-yl]-2,6-diazaspiro[3.5]nonane

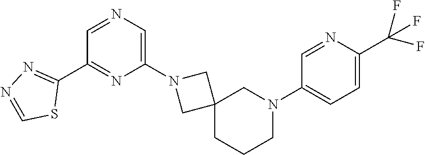 [SMILES: FC(F)(F)c1ccc(N2CCCC3(C2)CN(c2cncc(-c4nncs4)n2)C3)cn1]

Followed the General Procedure A to afford the desired product as a yellow solid (31 mg, 73%). ¹H NMR (500 MHz, DMSO-d₆) δ 9.71 (s, 1H), 8.65 (s, 1H), 8.50 (d, J=2.9 Hz, 1H), 8.07 (s, 1H), 7.63 (d, J=8.9 Hz, 1H), 7.53 (dd, J=8.9, 2.9 Hz, 1H), 3.92 (d, J=8.4 Hz, 2H), 3.86 (d, J=8.4 Hz, 2H), 3.57 (s, 2H), 3.30 (t, J=5.6 Hz, 2H), 1.88 (t, J=5.9 Hz, 2H), 1.70 (q, J=5.7 Hz, 2H). MS m/z: 434 [M+H]⁺.

### Example 139: 2-[1-(2,2,2-trifluoroethyl)-1H-pyrazolo[3,4-b]pyrazin-6-yl]-8-[4-(trifluoromethyl)pyridin-2-yl]-2,8-diazaspiro[4.5]decane

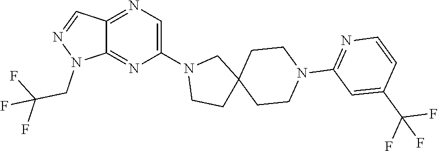 [SMILES: FC(F)(F)Cn1ncc2ncc(N3CCC4(CCN(c5cc(C(F)(F)F)ccn5)CC4)C3)nc21]

Followed the General Procedure A to afford the desired product as a white solid (40 mg, 88%). ¹H NMR (500 MHz, DMSO-d₆) δ 8.32 (d, J=5.1 Hz, 1H), 8.17 (s, 1H), 8.13 (s, 1H), 7.16-7.07 (m, 1H), 6.84 (dd, J=5.2, 1.4 Hz, 1H), 5.12 (q, J=9.1 Hz, 2H), 3.81-3.59 (m, 6H), 3.54 (s, 2H), 1.97 (s, 2H), 1.73-1.55 (m, 4H). MS m/z: 487 [M+H]⁺.

### Example 140: 2-[1-(2,2-difluoroethyl)-3-methyl-1H-pyrazolo[3,4-b]pyrazin-6-yl]-8-[4-(trifluoromethyl)pyridin-2-yl]-2,8-diazaspiro[4.5]decane

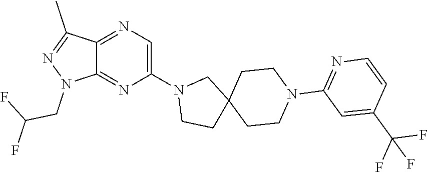 [SMILES: Cc1nn(CC(F)F)c2nc(N3CCC4(CCN(c5cc(C(F)(F)F)ccn5)CC4)C3)cnc12]

Followed the General Procedure A to afford the desired product as a white solid (30 mg, 67%). ¹H NMR (500 MHz, DMSO-d₆) δ 8.32 (d, J=5.1 Hz, 1H), 8.02 (s, 1H), 7.11 (s, 1H), 6.84 (dd, J=5.1, 1.4 Hz, 1H), 6.42 (tt, J=55.1, 4.0 Hz, 1H), 4.59 (td, J=14.8, 4.0 Hz, 2H), 3.68 (q, J=10.9, 7.1 Hz, 6H), 3.52 (s, 2H), 2.41 (s, 3H), 1.96 (d, J=8.1 Hz, 2H), 1.69-1.55 (m, 4H). MS m/z: 483 [M+H]⁺.

### Example 141: 2-[6-(1,3,4-thiadiazol-2-yl)pyrazin-2-yl]-8-[4-(trifluoromethyl)pyridin-2-yl]-2,8-diazaspiro[4.5]decane

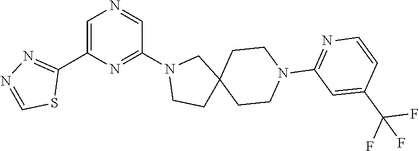 [SMILES: FC(F)(F)c1ccnc(N2CCC3(CC2)CCN(c2cncc(-c4nncs4)n2)C3)c1]

Followed the General Procedure A to afford the desired product as a white solid (25 mg, 60%). ¹H NMR (500 MHz, DMSO-d₆) δ 9.70 (s, 1H), 8.59 (s, 1H), 8.32 (d, J=5.1 Hz, 1H), 8.16 (s, 1H), 7.14-7.05 (m, 1H), 6.84 (dd, J=5.2, 1.4 Hz, 1H), 3.77-3.59 (m, 6H), 3.47 (s, 2H), 1.97 (t, J=7.0 Hz, 2H), 1.70-1.53 (m, 4H). MS m/z: 449 [M+H]⁺.

### Example 142: 8-[1-(2,2,2-trifluoroethyl)-1H-pyrazolo[3,4-b]pyrazin-6-yl]-2-[4-(trifluoromethyl)pyridin-2-yl]-2,8-diazaspiro[4.5]decan-1-one

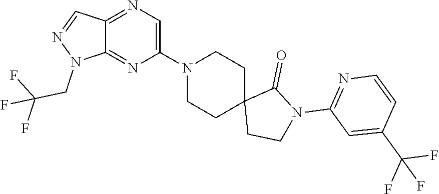 [SMILES: O=C1N(c2cc(C(F)(F)F)ccn2)CCC12CCN(c1cnc3cnn(CC(F)(F)F)c3n1)CC2]

Followed the General Procedure D to afford the desired product as a white solid (5.5 mg, 15%). ¹H NMR (500 MHz, DMSO-d⁶) δ 8.70 (d, J=5.4 Hz, 1H), 8.67 (dt, J=1.7, 0.8 Hz, 1H), 8.55 (s, 1H), 8.20 (s, 1H), 7.55-7.52 (m, 1H), 5.18 (q, J=9.1 Hz, 2H), 4.45 (d, J=13.8 Hz, 2H), 4.06 (dd, J=7.8, 6.3 Hz, 2H), 3.41-3.36 (m, 2H), 2.21 (t, J=7.1 Hz, 2H), 1.88-1.79 (m, 2H), 1.73 (d, J=13.4 Hz, 2H). MS m/z: 500 [M+H]⁺.

### Example 143: 8-[1-(2,2-difluoroethyl)-1H-pyrazolo[3,4-b]pyrazin-6-yl]-2-[6-(trifluoromethyl)pyridin-2-yl]-2,8-diazaspiro[4.5]decane

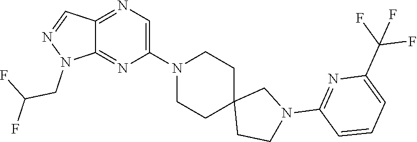 [SMILES: FC(F)Cn1ncc2ncc(N3CCC4(CCN(c5cccc(C(F)(F)F)n5)C4)CC3)nc21]

Followed the General Procedure A to afford the desired product as a light-yellow oil (30 mg, 70%). ¹H NMR (500 MHz, DMSO-d₆) δ 8.49 (s, 1H), 8.13 (s, 1H), 7.69 (dd, J=8.6, 7.3 Hz, 1H), 6.95 (d, J=7.2 Hz, 1H), 6.74 (d, J=8.6 Hz, 1H), 6.44 (tt, J=54.9, 3.8 Hz, 1H), 4.69 (td, J=15.0, 3.8 Hz, 2H), 3.82 (t, J=5.7 Hz, 4H), 3.52 (t, J=7.0 Hz, 2H), 3.38 (s, 2H), 1.95 (t, J=7.0 Hz, 2H), 1.73-1.61 (m, 4H). MS m/z: 468 [M+H]⁺.

### Example 144: 8-[1-(2,2-difluoroethyl)-1H-pyrazolo[3,4-b]pyrazin-6-yl]-2-[5-(trifluoromethyl)pyridin-2-yl]-2,8-diazaspiro[4.5]decane

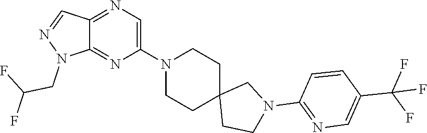 [SMILES: FC(F)Cn1ncc2ncc(N3CCC4(CCN(c5ccc(C(F)(F)F)cn5)C4)CC3)nc21]

Followed the General Procedure A to afford the desired product as a light-yellow oil (35 mg, 82%). ¹H NMR (500 MHz, DMSO-d₆) δ 8.49 (s, 1H), 8.40-8.35 (m, 1H), 8.13 (s, 1H), 7.76 (dd, J=9.0, 2.6 Hz, 1H), 6.59 (d, J=9.0 Hz, 1H), 6.44 (tt, J=54.9, 3.8 Hz, 1H), 4.69 (td, J=15.0, 3.8 Hz, 2H), 3.88 (ddd, J=13.4, 6.8, 4.4 Hz, 2H), 3.76 (ddd, J=13.5, 7.4, 4.3 Hz, 2H), 3.55 (s, 2H), 3.43 (s, 2H), 1.95 (t, J=7.0 Hz, 2H), 1.73-1.59 (m, 4H). MS m/z: 468 [M+H]⁺.

### Example 145: 8-[1-(2,2-difluoroethyl)-1H-pyrazolo[3,4-b]pyrazin-6-yl]-2-[3-(trifluoromethyl)pyridin-2-yl]-2,8-diazaspiro[4.5]decane

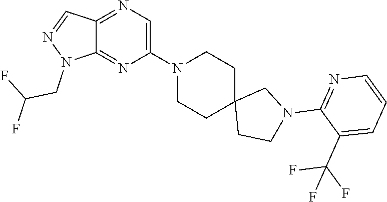 [SMILES: FC(F)Cn1ncc2ncc(N3CCC4(CC3)CCN(c3ncccc3C(F)(F)F)C4)nc21]

Followed the General Procedure A to afford the desired product as a light-yellow solid (35 mg, 75%). ¹H NMR (500 MHz, DMSO-d⁶) δ 8.48 (s, 1H), 8.34 (dd, J=4.7, 1.8 Hz, 1H), 8.13 (s, 1H), 7.93 (dd, J=7.8, 1.8 Hz, 1H), 6.81 (dd, J=7.8, 4.7 Hz, 1H), 6.44 (tt, J=54.9, 3.8 Hz, 1H), 4.69 (td, J=15.0, 3.8 Hz, 2H), 3.89-3.71 (m, 4H), 3.62 (t, J=7.0 Hz, 2H), 3.45 (s, 2H), 1.92 (t, J=7.1 Hz, 2H), 1.71-1.56 (m, 4H). MS m/z: 468 [M+H]⁺.

### Example 146: 8-[1-(2,2-difluoroethyl)-1H-pyrazolo[3,4-b]pyrazin-6-yl]-2-[2-(trifluoromethyl)pyridin-3-yl]-2,8-diazaspiro[4.5]decane

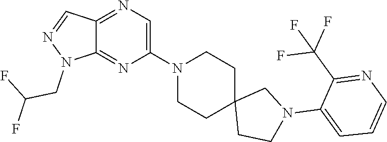 [SMILES: FC(F)Cn1ncc2ncc(N3CCC4(CC3)CCN(c3cccnc3C(F)(F)F)C4)nc21]

Followed the General Procedure A to afford the desired product as a light-yellow oil (24 mg, 56%). ¹H NMR (500 MHz, DMSO-d₆) δ 8.48 (s, 1H), 8.13 (s, 1H), 8.08 (dd, J=4.1, 1.4 Hz, 1H), 7.51 (dd, J=8.7, 1.4 Hz, 1H), 7.48 (dd, J=8.6, 4.0 Hz, 1H), 6.44 (tt, J=54.9, 3.8 Hz, 1H), 4.69 (td, J=15.0, 3.8 Hz, 2H), 3.81 (q, J=5.2 Hz, 4H), 3.45 (t, J=7.0 Hz, 2H), 3.24 (s, 2H), 1.92 (t, J=7.0 Hz, 2H), 1.67 (dt, J=7.5, 4.3 Hz, 4H). MS m/z: 468 [M+H]⁺.

### Example 147: 8-[1-(2,2-difluoroethyl)-1H-pyrazolo[3,4-b]pyrazin-6-yl]-2-[6-(trifluoromethyl)pyridin-3-yl]-2,8-diazaspiro[4.5]decane

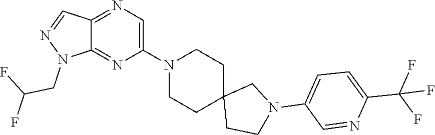 [SMILES: FC(F)Cn1ncc2ncc(N3CCC4(CCN(c5ccc(C(F)(F)F)nc5)C4)CC3)nc21]

Followed the General Procedure A to afford the desired product as a light-yellow solid (30 mg, 70%). ¹H NMR (500 MHz, DMSO-d₆) δ 8.50 (s, 1H), 8.13 (s, 1H), 8.04 (d, J=2.8 Hz, 1H), 7.59 (d, J=8.8 Hz, 1H), 7.00 (dd, J=8.8, 2.8 Hz, 1H), 6.44 (tt, J=54.9, 3.8 Hz, 1H), 4.69 (td, J=15.0, 3.8 Hz, 2H), 3.90 (ddd, J=13.5, 6.6, 4.3 Hz, 2H), 3.76 (ddd, J=13.5, 7.4, 4.2 Hz, 2H), 3.45 (t, J=7.0 Hz, 2H), 3.33 (s, 3H), 1.97 (t, J=7.0 Hz, 2H), 1.74-1.58 (m, 4H). MS m/z: 468 [M+H]⁺.

### Example 148: 8-[1-(2,2-difluoroethyl)-1H-pyrazolo[3,4-b]pyrazin-6-yl]-2-[5-(trifluoromethyl)pyridin-3-yl]-2,8-diazaspiro[4.5]decane

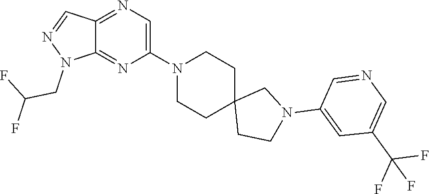 [SMILES: FC(F)Cn1ncc2ncc(N3CCC4(CCN(c5cncc(C(F)(F)F)c5)C4)CC3)nc21]

Followed the General Procedure A to afford the desired product as a light-yellow solid (26 mg, 61%). ¹H NMR (500 MHz, DMSO-d₆) δ 8.50 (s, 1H), 8.20 (d, J=2.7 Hz, 1H), 8.13 (s, 1H), 8.13-8.10 (m, 1H), 7.11 (t, J=2.4 Hz, 1H), 6.44 (tt, J=54.9, 3.8 Hz, 1H), 4.69 (td, J=15.0, 3.8 Hz, 2H), 3.93 (ddd, J=13.6, 6.5, 4.2 Hz, 2H), 3.73 (ddd, J=13.5, 7.9, 4.1 Hz, 2H), 3.45 (t, J=7.0 Hz, 2H), 3.31 (s, 2H), 1.95 (t, J=7.0 Hz, 2H), 1.73-1.59 (m, 4H). MS m/z: 468 [M+H]⁺.

### Example 149: 2-[1-(2,2-difluoroethyl)-1H-pyrazolo[3,4-b]pyrazin-6-yl]-8-[6-(trifluoromethyl)pyridin-2-yl]-2,8-diazaspiro[4.5]decane

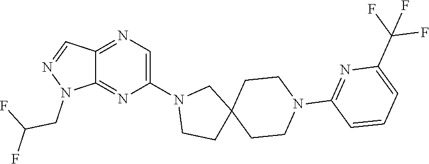 [SMILES: FC(F)Cn1ncc2ncc(N3CCC4(CCN(c5cccc(C(F)(F)F)n5)CC4)C3)nc21]

Followed the General Procedure A to afford the desired product as a white solid (26 mg, 61%). ¹H NMR (500 MHz, DMSO-d₆) δ 8.12 (s, 1H), 8.11 (s, 1H), 7.73 (ddd, J=8.8, 7.2, 0.8 Hz, 1H), 7.16 (d, J=8.8 Hz, 1H), 7.01 (d, J=7.2 Hz, 1H), 6.45 (tt, J=55.0, 3.9 Hz, 1H), 4.68 (td, J=14.9, 3.9 Hz, 2H), 3.67 (dt, J=19.8, 10.3 Hz, 6H), 3.54 (s, 2H), 1.98 (d, J=8.5 Hz, 2H), 1.73-1.56 (m, 4H). MS m/z: 468 [M+H]⁺.

### Example 150: 2-[1-(2,2-difluoroethyl)-1H-pyrazolo[3,4-b]pyrazin-6-yl]-8-[5-(trifluoromethyl)pyridin-2-yl]-2,8-diazaspiro[4.5]decane

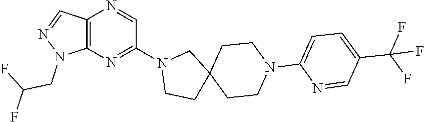 [SMILES: FC(F)Cn1ncc2ncc(N3CCC4(CCN(c5ccc(C(F)(F)F)cn5)CC4)C3)nc21]

Followed the General Procedure A to afford the desired product as a white solid (31 mg, 72%). ¹H NMR (500 MHz, DMSO-d₆) δ 8.40 (dt, J=2.8, 0.9 Hz, 1H), 8.12 (s, 1H), 8.10 (s, 1H), 7.77 (dd, J=9.2, 2.6 Hz, 1H), 6.99 (d, J=9.1 Hz, 1H), 6.45 (tt, J=55.0, 3.9 Hz, 2H), 4.68 (td, J=14.9, 3.9 Hz, 2H), 3.86-3.63 (m, 6H), 3.54 (s, 2H), 1.98 (d, J=8.7 Hz, 2H), 1.69-1.56 (m, 4H). MS m/z: 468 [M+H]⁺.

### Example 151: 2-[1-(2,2-difluoroethyl)-1H-pyrazolo[3,4-b]pyrazin-6-yl]-8-[3-(trifluoromethyl)pyridin-2-yl]-2,8-diazaspiro[4.5]decane

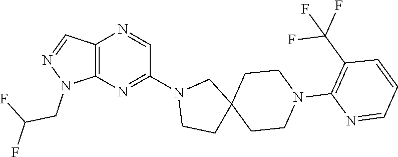 [SMILES: FC(F)Cn1ncc2ncc(N3CCC4(CCN(c5ncccc5C(F)(F)F)CC4)C3)nc21]

Followed the General Procedure A to afford the desired product as a white foam (33 mg, 77%). ¹H NMR (500 MHz, DMSO-d₆) δ 8.52 (dd, J=4.9, 1.8 Hz, 1H), 8.12 (s, 2H), 8.06 (dd, J=7.8, 1.9 Hz, 1H), 7.18 (ddd, J=7.8, 4.8, 0.9 Hz, 1H), 6.46 (tt, J=54.9, 3.9 Hz, 1H), 4.68 (td, J=14.9, 3.9 Hz, 2H), 3.67 (t, J=7.0 Hz, 2H), 3.54 (s, 2H), 3.29-3.18 (m, 4H), 1.96 (d, J=7.6 Hz, 2H), 1.78-1.63 (m, 4H). MS m/Z: 468 [M+H]⁺.

### Example 152: 2-[1-(2,2-difluoroethyl)-1H-pyrazolo[3,4-b]pyrazin-6-yl]-8-[6-(trifluoromethyl)pyridin-3-yl]-2,8-diazaspiro[4.5]decane

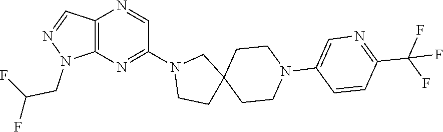 [SMILES: FC(F)Cn1ncc2ncc(N3CCC4(CCN(c5ccc(C(F)(F)F)nc5)CC4)C3)nc21]

Followed the General Procedure A to afford the desired product as a white solid (33 mg, 77%). ¹H NMR (500 MHz, DMSO-d₆) δ 8.46 (d, J=2.9 Hz, 1H), 8.12 (s, 1H), 8.11 (s, 1H), 7.63 (d, J=8.8 Hz, 1H), 7.46 (dd, J=8.9, 2.9 Hz, 1H), 6.45 (tt, J=55.0, 3.9 Hz, 1H), 4.68 (td, J=14.9, 3.9 Hz, 2H), 3.68 (t, J=7.0 Hz, 2H), 3.58-3.39 (m, 6H), 1.98 (d, J=8.5 Hz, 2H), 1.80-1.62 (m, 4H). MS m/z: 468 [M+H]⁺.

### Example 153: 2-[1-(2,2-difluoroethyl)-1H-pyrazolo[3,4-b]pyrazin-6-yl]-8-[5-(trifluoromethyl)pyridin-3-yl]-2,8-diazaspiro[4.5]decane

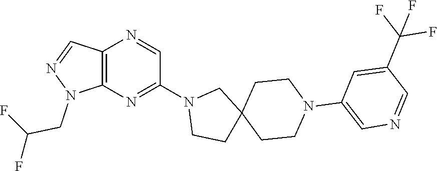 [SMILES: FC(F)Cn1ncc2ncc(N3CCC4(CCN(c5cncc(C(F)(F)F)c5)CC4)C3)nc21]

Followed the General Procedure A to afford the desired product as a white solid (33 mg, 77%). ¹H NMR (500 MHz, DMSO-d₆) δ 8.62 (d, J=2.8 Hz, 1H), 8.30-8.22 (m, 1H), 8.12 (s, 1H), 8.11 (s, 1H), 7.59 (t, J=2.4 Hz, 1H), 6.45 (tt, J=54.9, 3.9 Hz, 1H), 4.68 (td, J=14.9, 3.9 Hz, 2H), 3.68 (t, J=7.0 Hz, 2H), 3.53 (s, 2H), 3.49-3.36 (m, 4H), 1.97 (s, 2H), 1.75-1.66 (m, 4H). MS m/z: 468 [M+H]⁺.

### Example 154: 8-[1-(2,2-difluoroethyl)-1H-pyrazolo[3,4-b]pyrazin-6-yl]-2-[2-(trifluoromethyl)pyridin-4-yl]-2,8-diazaspiro[4.5]decane

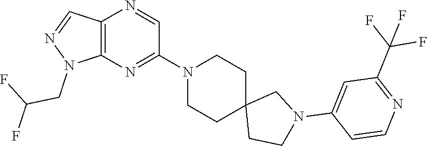 [SMILES: FC(F)Cn1ncc2ncc(N3CCC4(CCN(c5ccnc(C(F)(F)F)c5)C4)CC3)nc21]

Followed the General Procedure A to afford the desired product as a white solid (23 mg, 54%). ¹H NMR (500 MHz, DMSO-d₆) δ 8.50 (s, 1H), 8.21 (d, J=5.8 Hz, 1H), 8.13 (s, 1H), 6.86 (d, J=2.4 Hz, 1H), 6.68 (dd, J=5.9, 2.4 Hz, 1H), 6.44 (tt, J=54.9, 3.8 Hz, 1H), 4.69 (td, J=15.0, 3.8 Hz, 2H), 3.90 (t, J=9.7 Hz, 2H), 3.77-3.71 (m, 2H), 3.45 (t, J=7.0 Hz, 2H), 3.32 (s, 2H), 1.96 (t, J=7.0 Hz, 2H), 1.74-1.59 (m, 4H). MS m/z: 468 [M+H]⁺.

### Example 155: 2-[1-(2,2-difluoroethyl)-1H-pyrazolo[3,4-b]pyrazin-6-yl]-8-[2-(trifluoromethyl)pyridin-4-yl]-2,8-diazaspiro[4.5]decane

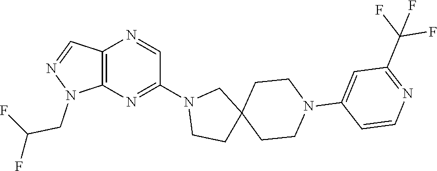 [SMILES: FC(F)Cn1ncc2ncc(N3CCC4(CCN(c5ccnc(C(F)(F)F)c5)CC4)C3)nc21]

Followed the General Procedure A to afford the desired product as a colorless oil (21 mg, 21%). ¹H NMR (500 MHz, DMSO-d₆) δ 8.26 (d, J=5.9 Hz, 1H), 8.12 (s, 1H), 8.10 (s, 1H), 7.23 (d, J=2.6 Hz, 1H), 7.07 (dd, J=6.1, 2.6 Hz, 1H), 6.45 (tt, J=55.0, 3.9 Hz, 2H), 4.68 (td, J=14.9, 3.9 Hz, 2H), 3.68 (t, J=7.0 Hz, 2H), 3.63-3.44 (m, 6H), 2.03-1.92 (m, 2H), 1.73-1.56 (m, 4H). MS m/z: 468 [M+H]⁺.

### Example 156: 8-[1-(2,2-difluoroethyl)-1H-pyrazolo[3,4-b]pyrazin-6-yl]-2-[3-(trifluoromethyl)pyridin-4-yl]-2,8-diazaspiro[4.5]decane

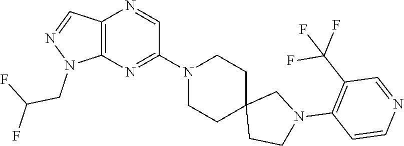 [SMILES: FC(F)Cn1ncc2ncc(N3CCC4(CC3)CCN(c3ccncc3C(F)(F)F)C4)nc21]

Followed the General Procedure A to afford the desired product as a colorless oil (10 mg, 69%). ¹H NMR (500 MHz, DMSO-d₆) δ 8.50 (s, 1H), 8.49 (s, 1H), 8.27 (d, J=6.2 Hz, 1H), 8.13 (s, 1H), 6.82 (d, J=6.2 Hz, 2H), 6.44 (tt, J=54.9, 3.5 Hz, 3H), 4.69 (td, J=15.0, 3.8 Hz, 3H), 3.81 (h, J=8.0 Hz, 4H), 3.57 (t, J=6.9 Hz, 2H), 3.36 (s, 2H), 1.96 (t, J=7.0 Hz, 2H), 1.74-1.59 (m, 4H). MS m/z: 468 [M+H]⁺.

### Example 157: 2-[1-(2,2-difluoroethyl)-1H-pyrazolo[3,4-b]pyrazin-6-yl]-6-[6-(difluoromethyl)pyridin-3-yl]-2,6-diazaspiro[3.5]nonane

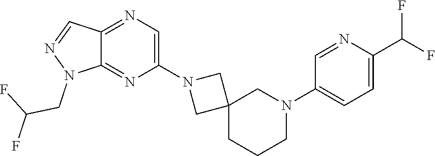 [SMILES: FC(F)Cn1ncc2ncc(N3CC4(CCCN(c5ccc(C(F)F)nc5)C4)C3)nc21]

Followed the General Procedure A to afford the desired product as a colorless oil (32 mg, 62%). ¹H NMR (500 MHz, DMSO-d⁶) δ 8.43 (d, J=2.8 Hz, 1H), 8.14 (s, 1H), 7.95 (s, 1H), 7.52 (dd, J=8.8, 2.8 Hz, 1H), 7.48 (d, J=8.8 Hz, 1H), 6.82 (t, J=55.4 Hz, 2H), 6.43 (tt, J=54.9, 3.8 Hz, 1H), 4.66 (td, J=15.0, 3.8 Hz, 2H), 3.94 (d, J=26.5 Hz, 4H), 3.49 (s, 2H), 3.23 (s, 2H), 1.86 (t, J=6.0 Hz, 2H), 1.69 (s, 2H). MS m/z: 436 [M+H]⁺.

### Example 158: 6-[6-(difluoromethyl)pyridin-3-yl]-2-[1-(2,2,2-trifluoroethyl)-1H-pyrazolo[3,4-b]pyrazin-6-yl]-2,6-diazaspiro[3.5]nonane

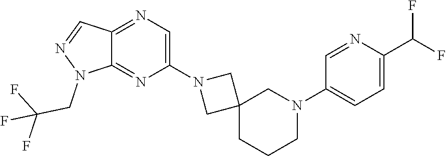 [SMILES: FC(F)c1ccc(N2CCCC3(C2)CN(c2cnc4cnn(CC(F)(F)F)c4n2)C3)cn1]

Followed the General Procedure A to afford the desired product as a light brown oil (34 mg, 63%). ¹H NMR (500 MHz, DMSO-d₆) δ 8.43 (d, J=2.8 Hz, 1H), 8.14 (s, 1H), 7.95 (s, 1H), 7.52 (dd, J=8.8, 2.8 Hz, 1H), 7.48 (d, J=8.8 Hz, 1H), 6.82 (t, J=55.4 Hz, 2H), 6.43 (tt, J=54.9, 3.8 Hz, 1H), 4.66 (td, J=15.0, 3.8 Hz, 2H), 3.94 (d, J=26.5 Hz, 4H), 3.49 (s, 2H), 3.23 (s, 2H), 1.86 (t, J=6.0 Hz, 2H), 1.69 (s, 2H). MS m/z: 454 [M+H]⁺.

### Example 159: 2-[1-(2,2-difluoroethyl)-3-methyl-1H-pyrazolo[3,4-b]pyrazin-6-yl]-6-[6-(difluoromethyl)pyridin-3-yl]-2,6-diazaspiro[3.5]nonane

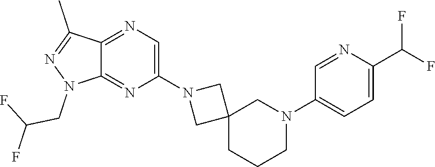 [SMILES: Cc1nn(CC(F)F)c2nc(N3CC4(CCCN(c5ccc(C(F)F)nc5)C4)C3)cnc12]

Followed the General Procedure A to afford the desired product as a white solid (31 mg, 58%). ¹H NMR (500 MHz, DMSO-d₆) δ 8.43 (d, J=2.8 Hz, 1H), 8.14 (s, 1H), 7.95 (s, 1H), 7.52 (dd, J=8.8, 2.8 Hz, 1H), 7.48 (d, J=8.8 Hz, 1H), 6.82 (t, J=55.4 Hz, 2H), 6.43 (tt, J=54.9, 3.8 Hz, 1H), 4.66 (td, J=15.0, 3.8 Hz, 2H), 3.94 (d, J=26.5 Hz, 4H), 3.49 (s, 2H), 3.23 (s, 2H), 1.86 (t, J=6.0 Hz, 2H), 1.69 (s, 2H). MS m/z: 451 [M+H]⁺.

### Example 160: 6-[6-(difluoromethyl)pyridin-3-yl]-2-[6-(1,3,4-thiadiazol-2-yl)pyrazin-2-yl]-2,6-diazaspiro[3.5]nonane

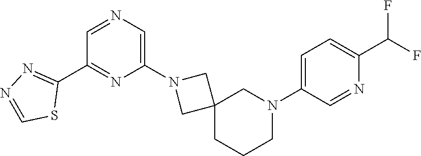 [SMILES: FC(F)c1ccc(N2CCCC3(C2)CN(c2cncc(-c4nncs4)n2)C3)cn1]

Followed the General Procedure A to afford the desired product as a yellow solid (33 mg, 67%). ¹H NMR (500 MHz, DMSO-d₆) δ 9.71 (s, 1H), 8.64 (s, 1H), 8.43 (d, J=2.6 Hz, 1H), 8.07 (s, 1H), 7.52 (dd, J=8.8, 2.8 Hz, 1H), 7.48 (d, J=8.7 Hz, 1H), 6.82 (t, J=55.4 Hz, 1H), 3.92 (d, J=8.4 Hz, 2H), 3.86 (d, J=8.4 Hz, 2H), 3.49 (s, 2H), 3.23 (t, J=5.5 Hz, 2H), 1.86 (t, J=5.9 Hz, 2H), 1.73-1.64 (m, 2H). MS m/z: 417 [M+H]⁺.

### Example 161: 2-[1-(2,2-difluoroethyl)-1H-pyrazolo[3,4-b]pyrazin-6-yl]-8-[4-(difluoromethyl)pyridin-2-yl]-2,8-diazaspiro[4.5]decane

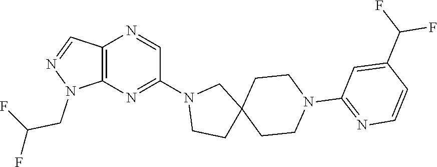 [SMILES: FC(F)Cn1ncc2ncc(N3CCC4(CCN(c5cc(C(F)F)ccn5)CC4)C3)nc21]

Followed the General Procedure A to afford the desired product as a colorless oil (24 mg, 59%). ¹H NMR (500 MHz, DMSO-d₆) δ 8.23 (d, J=5.1 Hz, 1H), 8.12 (s, 1H), 8.11 (s, 1H), 7.00 (s, 1H), 6.92 (t, J=55.5 Hz, 1H), 6.73 (d, J=5.1 Hz, 1H), 6.45 (tt, J=55.0, 3.9 Hz, 1H), 4.68 (td, J=14.9, 3.9 Hz, 2H), 3.68 (t, J=7.0 Hz, 4H), 3.62 (d, J=6.8 Hz, 2H), 3.53 (s, 2H), 1.96 (s, 2H), 1.68-1.55 (m, 4H). MS m/z: 451 [M+H]⁺.

### Example 162: 8-[4-(difluoromethyl)pyridin-2-yl]-2-[1-(2,2,2-trifluoroethyl)-1H-pyrazolo[3,4-b]pyrazin-6-yl]-2,8-diazaspiro[4.5]decane

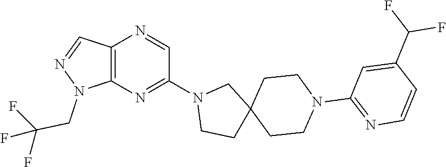 [SMILES: FC(F)c1ccnc(N2CCC3(CC2)CCN(c2cnc4cnn(CC(F)(F)F)c4n2)C3)c1]

Followed the General Procedure A to afford the desired product as a white solid (25 mg, 59%). ¹H NMR (500 MHz, DMSO-d₆) δ 8.23 (d, J=5.1 Hz, 1H), 8.18 (s, 1H), 8.13 (s, 1H), 7.00 (s, 1H), 6.92 (t, J=55.5 Hz, 1H), 6.76-6.70 (m, 1H), 5.13 (q, J=9.1 Hz, 2H), 3.68 (t, J=6.8 Hz, 4H), 3.61 (s, 2H), 3.54 (s, 2H), 1.97 (s, 2H), 1.71-1.54 (m, 4H). MS m/z: 468 [M+H]⁺.

### Example 163: 2-[1-(2,2-difluoroethyl)-3-methyl-1H-pyrazolo[3,4-b]pyrazin-6-yl]-8-[4-(difluoromethyl)pyridin-2-yl]-2,8-diazaspiro[4.5]decane

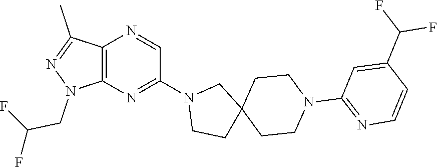 [SMILES: Cc1nn(CC(F)F)c2nc(N3CCC4(CCN(c5cc(C(F)F)ccn5)CC4)C3)cnc12]

Followed the General Procedure A to afford the desired product as a white solid (24 mg, 57%). ¹H NMR (500 MHz, DMSO-d₆) δ 8.23 (d, J=5.1 Hz, 1H), 8.02 (s, 1H), 7.00 (s, 1H), 6.92 (t, J=55.5 Hz, 1H), 6.73 (dd, J=5.1, 1.2 Hz, 1H), 6.42 (tt, J=55.0, 3.9 Hz, 1H), 4.59 (td, J=14.8, 4.0 Hz, 2H), 3.65 (dd, J=14.4, 7.3 Hz, 6H), 3.52 (s, 2H), 1.96 (s, 2H), 1.69-1.55 (m, 4H). MS m/z: 465 [M+H]⁺.

### Example 164: 8-[4-(difluoromethyl)pyridin-2-yl]-2-[6-(1,3,4-thiadiazol-2-yl)pyrazin-2-yl]-2,8-diazaspiro[4.5]decane

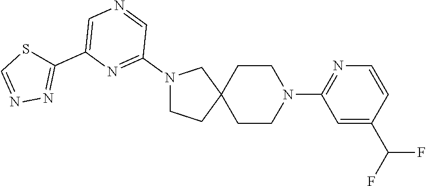 [SMILES: FC(F)c1ccnc(N2CCC3(CC2)CCN(c2cncc(-c4nncs4)n2)C3)c1]

Followed the General Procedure A to afford the desired product as a yellow solid (19 mg, 49%). ¹H NMR (500 MHz, DMSO-d₆) δ 9.71 (s, 1H), 8.59 (s, 1H), 8.23 (d, J=5.1 Hz, 1H), 8.16 (s, 1H), 7.00 (s, 1H), 6.92 (t, J=55.5 Hz, 1H), 6.73 (dd, J=5.2, 1.1 Hz, 1H), 3.67 (s, 2H), 3.62 (t, J=7.0 Hz, 4H), 3.47 (s, 2H), 1.96 (s, 2H), 1.72-1.54 (m, 4H). MS m/z: 431 [M+H]⁺.

### Example 165: 8-[1-(2,2-difluoroethyl)-1H-pyrazolo[3,4-b]pyrazin-6-yl]-2-[4-(difluoromethyl)pyridin-2-yl]-2,8-diazaspiro[4.5]decan-1-one

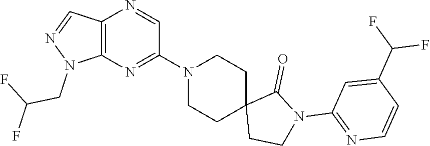 [SMILES: O=C1N(c2cc(C(F)F)ccn2)CCC12CCN(c1cnc3cnn(CC(F)F)c3n1)CC2]

Followed the General Procedure A to afford the desired product as a white solid (10 mg, 30%). ¹H NMR (500 MHz, DMSO-d₆) δ 8.58 (dd, J=5.1, 0.8 Hz, 1H), 8.54 (d, J=1.2 Hz, 1H), 8.53 (s, 1H), 8.16 (s, 1H), 7.34 (dd, J=5.3, 1.4 Hz, 1H), 7.14 (t, J=55.1 Hz, 2H), 6.45 (tt, J=54.9, 3.8 Hz, 2H), 4.71 (td, J=15.0, 3.8 Hz, 2H), 4.44 (dt, J=13.7, 4.1 Hz, 2H), 4.05 (dd, J=7.7, 6.3 Hz, 2H), 3.38-3.32 (m, 2H), 2.20 (t, J=7.0 Hz, 2H), 1.83 (ddd, J=13.2, 11.2, 4.2 Hz, 2H), 1.75-1.63 (m, 2H). MS m/z: 464 [M+H]⁺.

### Example 166: 2-[4-(difluoromethyl)pyridin-2-yl]-8-[1-(2,2,2-trifluoroethyl)-1H-pyrazolo[3,4-b]pyrazin-6-yl]-2,8-diazaspiro[4.5]decan-1-one

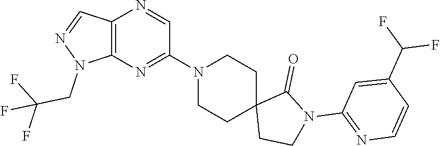 [SMILES: O=C1N(c2cc(C(F)F)ccn2)CCC12CCN(c1cnc3cnn(CC(F)(F)F)c3n1)CC2]

Followed the General Procedure A to afford the desired product as a white solid (21 mg, 60%). ¹H NMR (500 MHz, DMSO-d₆) δ 8.58 (dd, J=5.1, 0.8 Hz, 1H), 8.55 (s, 1H), 8.54 (t, J=1.2 Hz, 1H), 8.21 (s, 1H), 7.37-7.30 (m, 1H), 7.14 (t, J=55.1 Hz, 2H), 5.18 (q, J=9.1 Hz, 2H), 4.46 (dt, J=13.5, 4.2 Hz, 2H), 4.05 (dd, J=7.8, 6.2 Hz, 2H), 3.39-3.33 (m, 2H), 2.20 (t, J=7.0 Hz, 2H), 1.83 (ddd, J=13.3, 11.3, 4.2 Hz, 2H), 1.78-1.61 (m, 2H). MS m/z: 482 [M+H]⁺.

### Example 167: 8-[1-(2,2-difluoroethyl)-3-methyl-1H-pyrazolo[3,4-b]pyrazin-6-yl]-2-[4-(difluoromethyl)pyridin-2-yl]-2,8-diazaspiro[4.5]decan-1-one

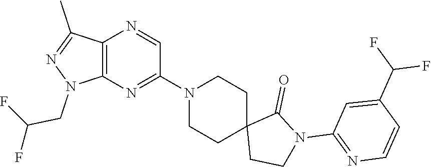 [SMILES: Cc1nn(CC(F)F)c2nc(N3CCC4(CC3)CCN(c3cc(C(F)F)ccn3)C4=O)cnc12]

Followed the General Procedure A to afford the desired product as a white solid (19 mg, 55%). ¹H NMR (500 MHz, DMSO-d₆) δ 8.58 (dd, J=5.2, 0.8 Hz, 1H), 8.54 (t, J=1.2 Hz, 1H), 8.44 (s, 1H), 7.34 (dd, J=5.2, 1.4 Hz, 1H), 7.14 (t, J=55.1 Hz, 2H), 6.42 (tt, J=55.0, 3.8 Hz, 1H), 4.62 (td, J=15.0, 3.9 Hz, 2H), 4.42 (dt, J=13.6, 4.1 Hz, 2H), 4.04 (dd, J=7.9, 6.1 Hz, 2H), 3.34-3.29 (m, 2H), 2.20 (t, J=7.0 Hz, 2H), 1.83 (ddd, J=13.1, 11.2, 4.2 Hz, 2H), 1.75-1.65 (m, 2H). MS m/z: 479 [M+H]⁺.

### Example 168: 8-[1-(2,2-difluoroethyl)-3-methyl-1H-pyrazolo[3,4-b]pyrazin-6-yl]-2-[4-(trifluoromethyl)pyridin-2-yl]-2,8-diazaspiro[4.5]decan-1-one

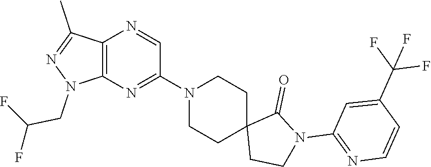 [SMILES: Cc1nn(CC(F)F)c2nc(N3CCC4(CC3)CCN(c3cc(C(F)(F)F)ccn3)C4=O)cnc12]

Followed the General Procedure A to afford the desired product as a white solid (29 mg, 66%). ¹H NMR (500 MHz, DMSO-d₆) δ 8.70 (dd, J=5.2, 1.0 Hz, 1H), 8.67 (dt, J=1.7, 0.8 Hz, 1H), 8.44 (s, 1H), 7.54 (dd, J=5.4, 1.6 Hz, 1H), 6.41 (tt, J=55.0, 3.9 Hz, 2H), 4.62 (td, J=14.9, 3.9 Hz, 2H), 4.41 (dt, J=13.6, 4.2 Hz, 2H), 4.06 (dd, J=7.9, 6.1 Hz, 2H), 3.40-3.34 (m, 2H), 2.42 (s, 3H), 2.21 (t, J=7.0 Hz, 2H), 1.84 (ddd, J=13.4, 11.2, 4.2 Hz, 2H), 1.77-1.67 (m, 2H). MS m/z: 496 [M+H]⁺.

### Example 169: 8-[6-(1,3,4-thiadiazol-2-yl)pyrazin-2-yl]-2-[4-(trifluoromethyl)pyridin-2-yl]-2,8-diazaspiro[4.5]decan-1-one

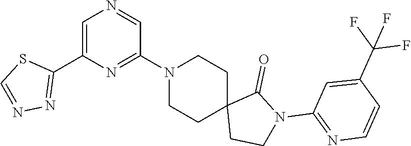 [SMILES: O=C1N(c2cc(C(F)(F)F)ccn2)CCC12CCN(c1cncc(-c3nncs3)n1)CC2]

Followed the General Procedure A to afford the desired product as a light-yellow solid (24 mg, 58%). ¹H NMR (500 MHz, DMSO-d₆) δ 9.72 (s, 1H), 8.70 (d, J=5.2 Hz, 1H), 8.67 (dt, J=1.5, 0.8 Hz, 1H), 8.64 (s, 1H), 8.59 (s, 1H), 7.54 (dd, J=5.3, 1.6 Hz, 1H), 4.32 (dt, J=13.7, 4.2 Hz, 2H), 4.06 (dd, J=7.7, 6.4 Hz, 2H), 3.33-3.26 (m, 2H), 2.21 (t, J=7.0 Hz, 2H), 1.84 (ddd, J=13.2, 11.2, 4.3 Hz, 2H), 1.78-1.69 (m, 2H). MS m/z: 463 [M+H]⁺.

### Example 170: 2-[1-(2,2-difluoroethyl)-1H-pyrazolo[3,4-b]pyrazin-6-yl]-8-[2-(trifluoromethyl)pyridin-3-yl]-2,8-diazaspiro[4.5]decane

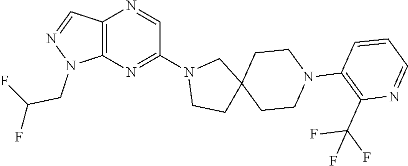 [SMILES: FC(F)Cn1ncc2ncc(N3CCC4(CCN(c5cccnc5C(F)(F)F)CC4)C3)nc21]

Followed the General Procedure A to afford the desired product as a light-yellow solid (29 mg, 80%). ¹H NMR (500 MHz, DMSO-d6) δ 8.45 (dd, J=4.5, 1.4 Hz, 1H), 8.13 (s, 1H), 8.12 (s, 1H), 8.08 (dd, J=8.2, 1.4 Hz, 1H), 7.70 (dd, J=8.3, 4.5 Hz, 1H), 6.46 (tt, J=54.9, 3.9 Hz, 1H), 4.69 (td, J=14.9, 3.9 Hz, 2H), 3.67 (t, J=7.0 Hz, 2H), 3.56 (s, 2H), 2.95 (h, J=7.5 Hz, 4H), 1.99 (s, 3H), 1.73 (h, J=8.5 Hz, 4H). MS m/z: 468 [M+H]⁺.

### Example 171: 2-[1-(2,2-difluoroethyl)-1H-pyrazolo[3,4-b]pyrazin-6-yl]-8-[4-(trifluoromethyl)pyridin-2-yl]-5-oxa-2,8-diazaspiro[3.5]nonane

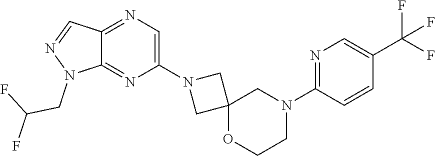 [SMILES: FC(F)Cn1ncc2ncc(N3CC4(CN(c5ccc(C(F)(F)F)cn5)CCO4)C3)nc21]

Followed the General Procedure A to afford the desired product as a colorless oil (4.5 mg, 17%). ¹H NMR (500 MHz, CDCl₃) δ 8.33 (d, J=5.1 Hz, 1H), 8.06 (s, 1H), 7.83 (s, 1H), 6.89 (dd, J=5.1, 1.3 Hz, 1H), 6.84 (s, 1H), 6.21 (tt, J=55.6, 4.5 Hz, 1H), 4.66 (td, J=13.3, 4.5 Hz, 2H), 4.21-4.13 (m, 4H), 3.92-3.88 (m, 2H), 3.87 (s, 2H), 3.60-3.52 (m, 2H). MS m/z: 456 [M+H]⁺.

### Example 172: 2-[1-(2,2-difluoroethyl)-1H-pyrazolo[3,4-b]pyrazin-6-yl]-8-[5-(trifluoromethyl)pyridin-2-yl]-5-oxa-2,8-diazaspiro[3.5]nonane

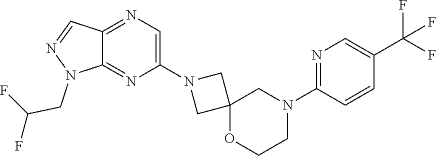 [SMILES: FC(F)Cn1ncc2ncc(N3CC4(CN(c5ccc(C(F)(F)F)cn5)CCO4)C3)nc21]

Followed the General Procedure A to afford the desired product as a colorless oil (12 mg, 46%). ¹H NMR (500 MHz, DMSO-d₆) δ 8.44 (dt, J=2.0, 0.9 Hz, 1H), 8.17 (s, 1H), 7.99 (s, 1H), 7.86 (dd, J=9.2, 2.6 Hz, 1H), 7.09 (d, J=9.1 Hz, 1H), 6.44 (tt, J=54.8, 3.8 Hz, 1H), 4.68 (td, J=15.0, 3.8 Hz, 2H), 4.18-4.05 (m, 4H), 3.91 (s, 2H), 3.79 (t, J=4.9 Hz, 2H), 3.65 (dd, J=6.1, 3.7 Hz, 2H). MS m/z: 456 [M+H]⁺.

### Example 173: 2-[1-(2,2-difluoroethyl)-1H-pyrazolo[3,4-b]pyrazin-6-yl]-8-[6-(trifluoromethyl)pyridin-3-yl]-5-oxa-2,8-diazaspiro[3.5]nonane

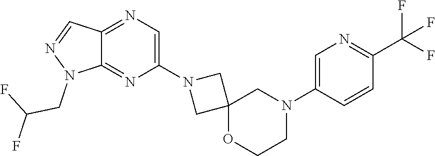 [SMILES: FC(F)Cn1ncc2ncc(N3CC4(CN(c5ccc(C(F)(F)F)nc5)CCO4)C3)nc21]

Followed the General Procedure A to afford the desired product as a white solid (5.2 mg, 20%). ¹H NMR (500 MHz, CDCl₃) δ 8.39 (d, J=2.9 Hz, 1H), 8.08 (s, 1H), 7.86 (s, 1H), 7.58 (d, J=8.7 Hz, 1H), 7.28 (dd, J=9.0, 3.2 Hz, 1H), 6.22 (tt, J=55.6, 4.5 Hz, 1H), 4.67 (td, J=13.3, 4.6 Hz, 2H), 4.19 (d, J=1.6 Hz, 4H), 3.97-3.92 (m, 2H), 3.51 (s, 2H), 3.35-3.28 (m, 2H). MS m/z: 456 [M+H]⁺.

### Example 174: 2-[1-(2,2-difluoroethyl)-1H-pyrazolo[3,4-b]pyrazin-6-yl]-9-[4-(trifluoromethyl)pyridin-2-yl]-6-oxa-2,9-diazaspiro[4.5]decane

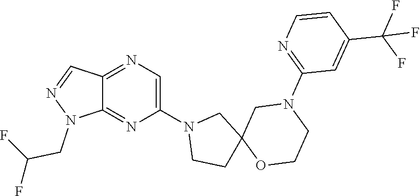 [SMILES: FC(F)Cn1ncc2ncc(N3CCC4(CN(c5cc(C(F)(F)F)ccn5)CCO4)C3)nc21]

Followed the General Procedure A to afford the desired product as a colorless oil (6.9 mg, 27%). ¹H NMR (500 MHz, CDCl₃) δ 8.32 (d, J=5.1 Hz, 1H), 8.04 (s, 1H), 7.95 (s, 1H), 6.87 (dd, J=5.1, 1.3 Hz, 1H), 6.83 (s, 1H), 6.21 (tt, J=55.7, 4.5 Hz, 1H), 4.65 (td, J=13.3, 4.5 Hz, 2H), 3.98-3.75 (m, 7H), 3.70-3.61 (m, 3H), 3.53 (ddd, J=12.7, 6.7, 4.0 Hz, 1H), 2.40-2.31 (m, 1H), 2.19 (dt, J=13.0, 8.0 Hz, 1H). MS m/z: 470 [M+H]⁺.

### Example 175: 2-[1-(2,2-difluoroethyl)-1H-pyrazolo[3,4-b]pyrazin-6-yl]-9-[5-(trifluoromethyl)pyridin-2-yl]-6-oxa-2,9-diazaspiro[4.5]decane

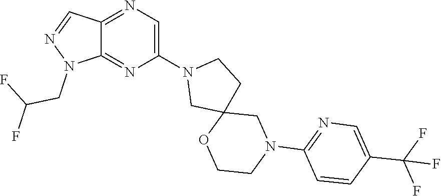 [SMILES: FC(F)Cn1ncc2ncc(N3CCC4(CN(c5ccc(C(F)(F)F)cn5)CCO4)C3)nc21]

Followed the General Procedure A to afford the desired product as a colorless oil (12 mg, 46%). ¹H NMR (500 MHz, DMSO-d₆) δ 8.43 (dt, J=2.7, 0.9 Hz, 1H), 8.13 (s, 1H), 8.11 (s, 1H), 7.85 (dd, J=9.2, 2.6 Hz, 1H), 7.06 (d, J=9.1 Hz, 1H), 6.43 (tt, J=54.9, 3.8 Hz, 1H), 4.67 (td, J=15.0, 3.8 Hz, 2H), 3.92-3.57 (m, 10H), 2.22 (s, 1H), 2.17-2.04 (m, 1H). MS m/z: 470 [M+H]⁺.

### Example 176: 2-[1-(2,2-difluoroethyl)-1H-pyrazolo[3,4-b]pyrazin-6-yl]-9-[6-(trifluoromethyl)pyridin-3-yl]-6-oxa-2,9-diazaspiro[4.5]decane

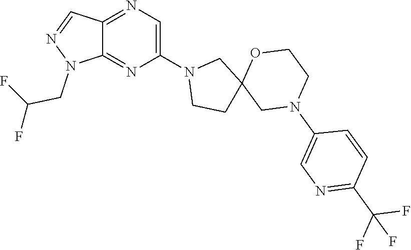 [SMILES: FC(F)Cn1ncc2ncc(N3CCC4(CN(c5ccc(C(F)(F)F)nc5)CCO4)C3)nc21]

Followed the General Procedure A to afford the desired product as a colorless oil (3.5 mg, 13%). ¹H NMR (500 MHz, CDCl₃) δ 8.38 (d, J=2.8 Hz, 1H), 8.05 (s, 1H), 7.98 (s, 1H), 7.57 (d, J=8.7 Hz, 1H), 7.24 (d, J=2.9 Hz, 1H), 6.22 (tt, J=55.7, 4.5 Hz, 1H), 4.67 (td, J=13.3, 4.5 Hz, 2H), 4.05-3.89 (m, 3H), 3.80 (td, J=10.5, 9.1, 4.6 Hz, 2H), 3.69 (d, J=12.0 Hz, 1H), 3.45-3.25 (m, 4H), 2.43 (ddd, J=12.6, 7.4, 4.7 Hz, 1H), 2.22-2.14 (m, 1H). MS m/z: 470 [M+H]⁺.

### Example 177: 8-[1-(2,2,2-trifluoroethyl)-1H-pyrazolo[3,4-b]pyrazin-6-yl]-2-[4-(trifluoromethyl)pyridin-2-yl]-2,8-diazaspiro[4.5]decane

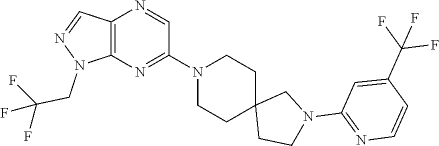 [SMILES: FC(F)(F)Cn1ncc2ncc(N3CCC4(CCN(c5cc(C(F)(F)F)ccn5)C4)CC3)nc21]

Followed the General Procedure A to afford the desired product as a colorless oil (33 mg, 73%). ¹H NMR (500 MHz, DMSO-d₆) δ 8.52 (s, 1H), 8.28 (d, J=5.2 Hz, 1H), 8.19 (s, 1H), 6.79 (dd, J=5.2, 1.5 Hz, 1H), 6.69 (s, 1H), 5.16 (q, J=9.1 Hz, 2H), 3.92 (ddd, J=13.6, 6.8, 4.2 Hz, 2H), 3.76 (ddd, J=13.6, 7.8, 4.0 Hz, 2H), 3.54 (t, J=6.9 Hz, 2H), 3.42 (s, 2H), 1.93 (q, J=8.1, 7.5 Hz, 2H), 1.66 (pt, J=7.7, 4.1 Hz, 4H). MS m/z: 486 [M+H]⁺.

### Example 178: 8-[1-(2,2-difluoroethyl)-3-methyl-1H-pyrazolo[3,4-b]pyrazin-6-yl]-2-[4-(trifluoromethyl)pyridin-2-yl]-2,8-diazaspiro[4.5]decane

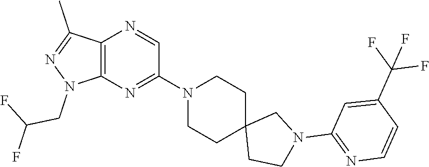 [SMILES: Cc1nn(CC(F)F)c2nc(N3CCC4(CCN(c5cc(C(F)(F)F)ccn5)C4)CC3)cnc12]

Followed the General Procedure A to afford the desired product as a colorless oil (33 mg, 74%). ¹H NMR (500 MHz, DMSO-d₆) δ 8.41 (s, 1H), 8.28 (d, J=5.2 Hz, 1H), 6.79 (dd, J=5.3, 1.5 Hz, 1H), 6.69 (s, 1H), 6.41 (tt, J=55.0, 3.9 Hz, 1H), 4.60 (td, J=15.0, 3.9 Hz, 2H), 3.89 (ddd, J=13.4, 6.7, 4.2 Hz, 2H), 3.72 (ddd, J=13.5, 7.8, 4.0 Hz, 2H), 3.54 (t, J=7.0 Hz, 2H), 3.42 (s, 2H), 2.41 (s, 3H), 1.93 (t, J=7.0 Hz, 2H), 1.66 (pt, J=7.7, 4.1 Hz, 4H). MS m/z: 483 [M+H]⁺.

### Example 179: 8-[1-(2,2-difluoroethyl)-5-methyl-1H-pyrazolo[3,4-b]pyrazin-6-yl]-2-[4-(trifluoromethyl)pyridin-2-yl]-2,8-diazaspiro[4.5]decane

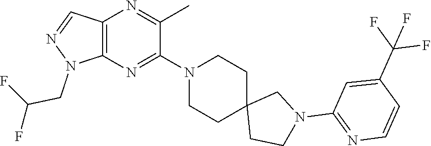 [SMILES: Cc1nc2cnn(CC(F)F)c2nc1N1CCC2(CCN(c3cc(C(F)(F)F)ccn3)C2)CC1]

Followed the General Procedure A to afford the desired product as a colorless oil (30 mg, 81%). ¹H NMR (500 MHz, CDCl₃) δ 8.29 (d, J=5.2 Hz, 1H), 8.07 (s, 1H), 6.72 (dd, J=5.2, 1.4 Hz, 1H), 6.54 (s, 1H), 6.23 (tt, J=55.5, 4.5 Hz, 2H), 4.72 (td, J=13.3, 4.5 Hz, 2H), 3.60 (t, J=7.0 Hz, 2H), 3.50 (s, 2H), 3.42 (dt, J=13.2, 5.1 Hz, 2H), 3.29 (dt, J=12.9, 5.9 Hz, 2H), 2.65 (s, 3H), 2.03 (t, J=7.0 Hz, 2H), 1.85 (dd, J=6.5, 4.6 Hz, 4H). MS m/z: 483 [M+H]⁺.

### Example 180: 8-[6-(1,3,4-thiadiazol-2-yl)pyrazin-2-yl]-2-[4-(trifluoromethyl)pyridin-2-yl]-2,8-diazaspiro[4.5]decane

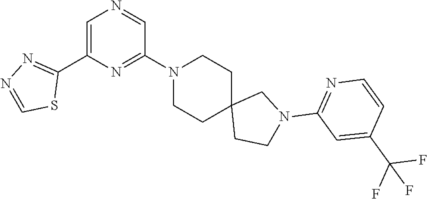 [SMILES: FC(F)(F)c1ccnc(N2CCC3(CCN(c4cncc(-c5nncs5)n4)CC3)C2)c1]

Followed the General Procedure A to afford the desired product as a yellow solid (29 mg, 70%). ¹H NMR (500 MHz, DMSO-d₆) δ 9.71 (s, 1H), 8.61 (s, 1H), 8.55 (s, 1H), 8.28 (d, J=5.2 Hz, 1H), 6.79 (dd, J=5.2, 1.5 Hz, 1H), 6.69 (s, 1H), 3.82 (ddd, J=13.4, 6.6, 4.2 Hz, 2H), 3.67 (ddd, J=13.4, 7.8, 4.1 Hz, 2H), 3.55 (t, J=7.0 Hz, 2H), 3.43 (s, 2H), 1.94 (t, J=7.0 Hz, 2H), 1.74-1.57 (m, 4H). MS m/z: 449 [M+H]⁺.

### Example 181: 2-[1-(2,2-difluoroethyl)-1H-pyrazolo[3,4-b]pyrazin-6-yl]-6-[4-(trifluoromethyl)pyridin-2-yl]-2,6-diazaspiro[3.4]octane

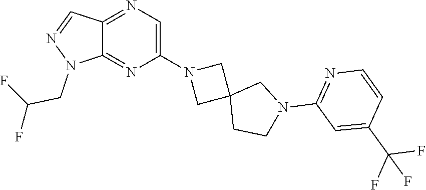 [SMILES: FC(F)Cn1ncc2ncc(N3CC4(CCN(c5cc(C(F)(F)F)ccn5)C4)C3)nc21]

Followed the General Procedure A to afford the desired product as a colorless oil (25 mg, 56%). ¹H NMR (500 MHz, CDCl₃) δ 8.29 (d, J=5.2 Hz, 1H), 8.06 (s, 1H), 7.81 (s, 1H), 6.79-6.73 (m, 1H), 6.56-6.52 (m, 1H), 6.22 (tt, J=55.6, 4.5 Hz, 1H), 4.66 (td, J=13.3, 4.5 Hz, 2H), 4.27-4.18 (m, 4H), 3.81 (s, 2H), 3.63 (t, J=6.8 Hz, 2H), 2.38 (t, J=6.9 Hz, 2H). MS m/z: 440 [M+H]⁺.

### Example 182: 2-[1-(2,2-difluoroethyl)-1H-pyrazolo[3,4-b]pyrazin-6-yl]-6-[5-(trifluoromethyl)pyridin-2-yl]-2,6-diazaspiro[3.4]octane

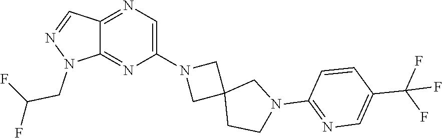 [SMILES: FC(F)Cn1ncc2ncc(N3CC4(CCN(c5ccc(C(F)(F)F)cn5)C4)C3)nc21]

Followed the General Procedure A to afford the desired product as a white solid (23 mg, 51%). ¹H NMR (500 MHz, CDCl₃) δ 8.40 (dq, J=2.5, 0.9 Hz, 1H), 8.07 (s, 1H), 7.81 (s, 1H), 7.66-7.61 (m, 1H), 6.40 (d, J=8.9 Hz, 1H), 6.22 (tt, J=55.6, 4.5 Hz, 1H), 4.66 (td, J=13.3, 4.5 Hz, 2H), 4.27-4.17 (m, 4H), 3.82 (s, 2H), 3.63 (t, J=6.9 Hz, 2H), 2.38 (t, J=6.9 Hz, 2H). MS m/z: 440 [M+H]⁺.

### Example 183: 2-[1-(2,2-difluoroethyl)-1H-pyrazolo[3,4-b]pyrazin-6-yl]-6-[6-(trifluoromethyl)pyridin-3-yl]-2,6-diazaspiro[3.4]octane

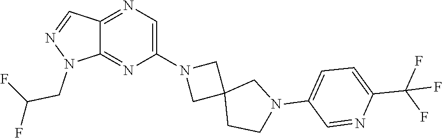 [SMILES: FC(F)Cn1ncc2ncc(N3CC4(CCN(c5ccc(C(F)(F)F)nc5)C4)C3)nc21]

Followed the General Procedure A to afford the desired product as a colorless oil (24 mg, 53%). ¹H NMR (500 MHz, CDCl₃) δ 8.07 (s, 1H), 8.04 (d, J=2.8 Hz, 1H), 7.82 (s, 1H), 7.51 (d, J=8.6 Hz, 1H), 6.86 (dd, J=8.7, 2.9 Hz, 1H), 6.22 (tt, J=55.6, 4.5 Hz, 1H), 4.66 (td, J=13.3, 4.5 Hz, 2H), 4.24 (q, J=9.0 Hz, 4H), 3.67 (s, 2H), 3.53 (t, J=6.8 Hz, 2H), 2.41 (t, J=6.9 Hz, 2H). MS m/z: 440 [M+H]⁺.

### Example 184: 2-[1-(2,2-difluoroethyl)-1H-pyrazolo[3,4-b]pyrazin-6-yl]-6-[5-(trifluoromethyl)pyridin-3-yl]-2,6-diazaspiro[3.4]octane

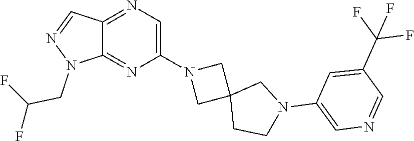 [SMILES: FC(F)Cn1ncc2ncc(N3CC4(CCN(c5cncc(C(F)(F)F)c5)C4)C3)nc21]

Followed the General Procedure A to afford the desired product as an off-white solid (24 mg, 53%). ¹H NMR (500 MHz, CDCl₃) δ 8.23 (s, 1H), 8.15 (d, J=2.8 Hz, 1H), 8.07 (s, 1H), 7.82 (s, 1H), 6.97 (t, J=2.4 Hz, 1H), 6.22 (tt, J=55.5, 4.4 Hz, 1H), 4.66 (td, J=13.3, 4.5 Hz, 2H), 4.24 (q, J=8.7 Hz, 4H), 3.67 (s, 2H), 3.53 (t, J=6.8 Hz, 2H), 2.41 (t, J=6.8 Hz, 2H). MS m/z: 440 [M+H]⁺.

### Example 185: 2-[1-(2,2-difluoroethyl)-1H-pyrazolo[3,4-b]pyrazin-6-yl]-6-[6-(trifluoromethyl)pyridin-2-yl]-2,6-diazaspiro[3.4]octane

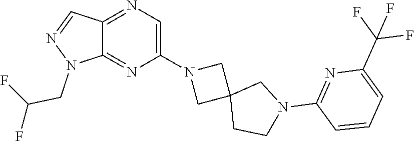 [SMILES: FC(F)Cn1ncc2ncc(N3CC4(CCN(c5cccc(C(F)(F)F)n5)C4)C3)nc21]

Followed the General Procedure A to afford the desired product as a white solid (23 mg, 51%). ¹H NMR (500 MHz, CDCl₃) δ 8.06 (s, 1H), 7.81 (s, 1H), 7.57 (ddd, J=8.6, 7.5, 0.8 Hz, 1H), 6.93 (d, J=7.3 Hz, 1H), 6.52 (d, J=8.5 Hz, 1H), 6.22 (tt, J=55.6, 4.5 Hz, 1H), 4.66 (td, J=13.3, 4.5 Hz, 2H), 4.24 (d, J=9.0 Hz, 2H), 4.19 (d, J=9.0 Hz, 2H), 3.82 (s, 2H), 3.62 (t, J=6.8 Hz, 2H), 2.36 (t, J=6.8 Hz, 2H). MS m/z: 440 [M+H]⁺.

### Example 186: 6-[1-(2,2-difluoroethyl)-1H-pyrazolo[3,4-b]pyrazin-6-yl]-2-[5-(trifluoromethyl)pyridin-2-yl]-2,6-diazaspiro[3.4]octane

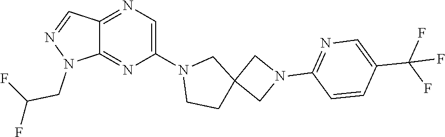 [SMILES: FC(F)Cn1ncc2ncc(N3CCC4(CN(c5ccc(C(F)(F)F)cn5)C4)C3)nc21]

Followed the General Procedure A to afford the desired product as a white solid (28 mg, 62%). ¹H NMR (500 MHz, CDCl₃) δ 8.40 (dt, J=2.0, 1.0 Hz, 1H), 8.06 (s, 1H), 7.97 (s, 1H), 7.67-7.59 (m, 1H), 6.32 (d, J=8.6 Hz, 1H), 6.35-6.09 (m, 1H), 4.67 (td, J=13.4, 4.5 Hz, 2H), 4.15 (d, J=8.3 Hz, 2H), 4.10 (d, J=8.3 Hz, 2H), 3.89 (s, 2H), 3.75 (t, J=6.9 Hz, 2H), 2.39 (t, J=6.9 Hz, 2H). MS m/z: 440 [M+H]⁺.

### Example 187: 6-[1-(2,2-difluoroethyl)-1H-pyrazolo[3,4-b]pyrazin-6-yl]-2-[4-(trifluoromethyl)pyridin-2-yl]-2,6-diazaspiro[3.4]octane

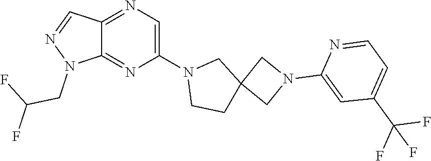 [SMILES: FC(F)Cn1ncc2ncc(N3CCC4(CN(c5cc(C(F)(F)F)ccn5)C4)C3)nc21]

Followed the General Procedure A to afford the desired product as a yellow oil (28 mg, 62%). ¹H NMR (500 MHz, CDCl₃) δ 8.30 (d, J=5.3 Hz, 1H), 8.06 (s, 1H), 7.97 (s, 1H), 6.86-6.79 (m, 1H), 6.50-6.44 (m, 1H), 6.23 (tt, J=55.7, 4.5 Hz, 1H), 4.67 (td, J=13.4, 4.5 Hz, 2H), 4.13 (d, J=8.1 Hz, 2H), 4.09 (d, J=8.1 Hz, 2H), 3.89 (s, 2H), 3.75 (t, J=6.9 Hz, 2H), 2.39 (t, J=6.9 Hz, 2H). MS m/z: 440 [M+H]⁺.

### Example 188: 7-[1-(2,2-difluoroethyl)-1H-pyrazolo[3,4-b]pyrazin-6-yl]-1-[2-(trifluoromethyl)pyridin-4-yl]-1,7-diazaspiro[4.4]nonane

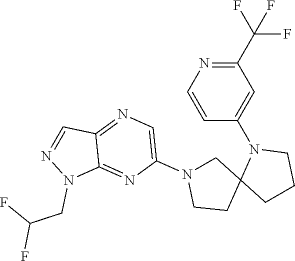 [SMILES: FC(F)Cn1ncc2ncc(N3CCC4(CCCN4c4ccnc(C(F)(F)F)c4)C3)nc21]

Followed the General Procedure A to afford the desired product as a light-yellow oil (11 mg, 62%). ¹H NMR (500 MHz, CDCl₃) δ 8.29 (d, J=6.0 Hz, 1H), 8.09 (s, 1H), 8.03 (s, 1H), 6.85 (d, J=2.5 Hz, 1H), 6.60 (dd, J=6.0, 2.5 Hz, 1H), 6.22 (tt, J=55.6, 4.5 Hz, 1H), 4.67 (tdd, J=13.4, 6.4, 4.5 Hz, 2H), 4.28 (d, J=12.0 Hz, 1H), 4.19 (ddd, J=10.9, 9.3, 1.5 Hz, 1H), 3.65-3.52 (m, 3H), 3.43 (d, J=12.0 Hz, 1H), 2.94 (ddd, J=13.2, 10.8, 9.3 Hz, 1H), 2.31-2.21 (m, 2H), 2.19-2.08 (m, 2H), 1.93-1.83 (m, 1H). MS m/z: 454 [M+H]⁺.

### Example 189: N-[1-(2,2-difluoroethyl)-1H-pyrazolo[3,4-b]pyrazin-6-yl]-2-[6-(trifluoromethyl)pyridin-3-yl]-2-azaspiro[3.4]octan-6-amine

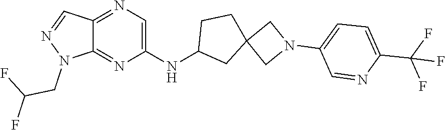 [SMILES: FC(F)Cn1ncc2ncc(NC3CCC4(C3)CN(c3ccc(C(F)(F)F)nc3)C4)nc21]

Followed the General Procedure A to afford the desired product as a white solid (11 mg, 33%). ¹H NMR (500 MHz, CDCl₃) δ 8.03 (s, 1H), 7.87 (d, J=2.8 Hz, 1H), 7.85 (s, 1H), 7.47 (d, J=8.5 Hz, 1H), 6.73 (dd, J=8.5, 2.8 Hz, 1H), 6.22 (tt, J=55.6, 4.4 Hz, 1H), 5.01 (d, J=6.2 Hz, 1H), 4.67 (td, J=13.4, 4.5 Hz, 2H), 4.39 (h, J=7.0 Hz, 1H), 3.97-3.90 (m, 4H), 2.57 (dd, J=13.6, 7.4 Hz, 1H), 2.34 (dtd, J=13.5, 7.7, 5.8 Hz, 1H), 2.16 (ddd, J=14.0, 8.5, 5.8 Hz, 1H), 2.06 (dt, J=13.4, 7.6 Hz, 1H), 1.91 (dd, J=13.6, 6.9 Hz, 1H), 1.65 (ddd, J=13.4, 8.5, 6.9 Hz, 1H). MS m/z: 454 [M+H]⁺.

### Example 190: N-[1-(2,2-difluoroethyl)-1H-pyrazolo[3,4-b]pyrazin-6-yl]-2-[4-(trifluoromethyl)pyridin-2-yl]-2-azaspiro[3.4]octan-6-amine

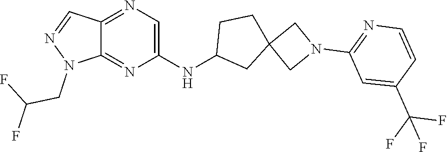 [SMILES: FC(F)Cn1ncc2ncc(NC3CCC4(C3)CN(c3cc(C(F)(F)F)ccn3)C4)nc21]

Followed the General Procedure A to afford the desired product as a light-yellow oil (12 mg, 12%). ¹H NMR (500 MHz, CDCl₃) δ 8.26 (d, J=5.3 Hz, 1H), 8.02 (s, 1H), 7.84 (s, 1H), 6.76 (dd, J=5.3, 1.4 Hz, 1H), 6.44 (s, 1H), 6.21 (tt, J=55.6, 4.5 Hz, 1H), 5.02 (d, J=6.3 Hz, 1H), 4.66 (td, J=13.4, 4.5 Hz, 2H), 4.40 (h, J=6.9 Hz, 1H), 4.05-3.96 (m, 4H), 2.55 (dd, J=13.5, 7.4 Hz, 1H), 2.34 (dtd, J=13.5, 7.7, 5.8 Hz, 1H), 2.15 (ddd, J=14.0, 8.5, 5.8 Hz, 1H), 2.05 (dt, J=13.3, 7.6 Hz, 1H), 1.90 (dd, J=13.6, 6.7 Hz, 1H), 1.64 (ddd, J=15.4, 13.7, 7.4 Hz, 2H). MS m/z: 454 [M+H]⁺.

### Example 191: N-[1-(2,2-difluoroethyl)-1H-pyrazolo[3,4-b]pyrazin-6-yl]-2-[6-(trifluoromethyl)pyridin-3-yl]-5-oxa-2-azaspiro[3.4]octan-7-amine

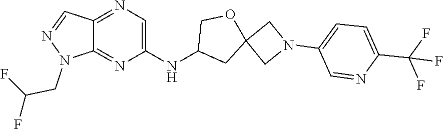 [SMILES: FC(F)Cn1ncc2ncc(NC3COC4(C3)CN(c3ccc(C(F)(F)F)nc3)C4)nc21]

Followed the General Procedure A to afford the desired product as a colorless oil (17 mg, 52%). ¹H NMR (500 MHz, CDCl₃) δ 8.06 (s, 1H), 7.90 (s, 1H), 7.89 (d, J=2.8 Hz, 1H), 7.48 (d, J=8.5 Hz, 1H), 6.76 (dd, J=8.5, 2.8 Hz, 1H), 6.21 (tt, J=55.6, 4.5 Hz, 1H), 5.19 (d, J=5.9 Hz, 1H), 4.68 (td, J=13.4, 4.4 Hz, 3H), 4.25 (dd, J=9.7, 5.6 Hz, 1H), 4.19-4.08 (m, 4H), 3.90 (dd, J=9.7, 3.5 Hz, 1H), 2.73 (dd, J=13.7, 6.9 Hz, 1H), 2.40 (dd, J=13.7, 4.2 Hz, 1H). MS m/z: 456 [M+H]⁺.

### Example 192: N-[1-(2,2-difluoroethyl)-1H-pyrazolo[3,4-b]pyrazin-6-yl]-2-[4-(trifluoromethyl)pyridin-2-yl]-5-oxa-2-azaspiro[3.4]octan-7-amine

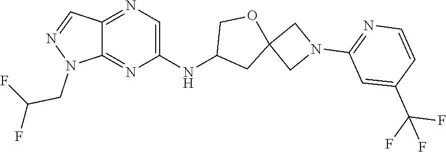 [SMILES: FC(F)Cn1ncc2ncc(NC3COC4(C3)CN(c3cc(C(F)(F)F)ccn3)C4)nc21]

Followed the General Procedure A to afford the desired product as a light-yellow solid (15 mg, 46%). ¹H NMR (500 MHz, CDCl₃) δ 8.27 (d, J=5.3 Hz, 1H), 8.05 (s, 1H), 7.89 (s, 1H), 6.80 (dd, J=5.3, 1.5 Hz, 1H), 6.49-6.44 (m, 1H), 6.21 (tt, J=55.5, 4.4 Hz, 1H), 5.17 (d, J=6.0 Hz, 1H), 4.73-4.63 (m, 3H), 4.29-4.20 (m, 3H), 4.16 (ddd, J=18.2, 8.6, 1.1 Hz, 2H), 3.91 (dd, J=9.7, 3.4 Hz, 1H), 2.71 (dd, J=13.7, 6.9 Hz, 1H), 2.38 (dd, J=13.7, 4.1 Hz, 1H). MS m/z: 456 [M+H]⁺.

### Example 193: N-[1-(2,2-difluoroethyl)-1H-pyrazolo[3,4-b]pyrazin-6-yl]-2-[6-(trifluoromethyl)pyridin-3-yl]-2-azaspiro[4.4]nonan-7-amine

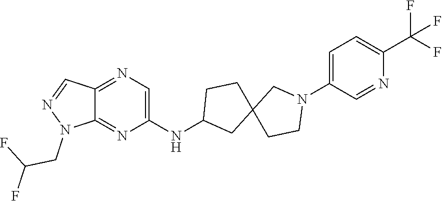 [SMILES: FC(F)Cn1ncc2ncc(NC3CCC4(CCN(c5ccc(C(F)(F)F)nc5)C4)C3)nc21]

Followed the General Procedure A to afford the desired product as a colorless oil (24 mg, 74%). ¹H NMR (500 MHz, CDCl₃) δ 8.02 (s, 1H), 7.97 (d, J=2.8 Hz, 1H), 7.85 (s, 1H), 7.46 (d, J=8.6 Hz, 1H), 6.79 (dd, J=8.7, 2.9 Hz, 1H), 6.22 (tt, J=55.6, 4.5 Hz, 1H), 5.16 (d, J=6.3 Hz, 1H), 4.66 (td, J=13.3, 4.5 Hz, 2H), 4.46 (dt, J=14.4, 7.3 Hz, 1H), 3.46 (t, J=6.9 Hz, 2H), 3.35-3.26 (m, 2H), 2.45-2.30 (m, 2H), 2.04 (t, J=6.8 Hz, 2H), 1.90 (ddd, J=13.4, 8.4, 5.4 Hz, 1H), 1.78 (dt, J=13.0, 7.6 Hz, 1H), 1.72-1.65 (m, 2H). MS m/z: 468 [M+H]⁺.

### Example 194: N-[1-(2,2-difluoroethyl)-1H-pyrazolo[3,4-b]pyrazin-6-yl]-2-[4-(trifluoromethyl)pyridin-2-yl]-2-azaspiro[4.4]nonan-7-amine

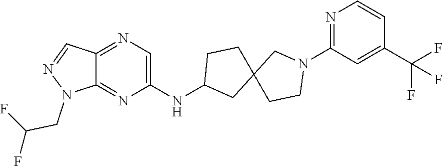 [SMILES: FC(F)Cn1ncc2ncc(NC3CCC4(CCN(c5cc(C(F)(F)F)ccn5)C4)C3)nc21]

Followed the General Procedure A to afford the desired product as a colorless oil (17 mg, 52%). ¹H NMR (500 MHz, CDCl₃) δ 8.26 (d, J=5.2 Hz, 1H), 8.01 (s, 1H), 7.84 (s, 1H), 6.70 (dd, J=5.3, 1.5 Hz, 1H), 6.50 (s, 1H), 6.22 (tt, J=55.6, 4.5 Hz, 1H), 5.10 (d, J=6.5 Hz, 1H), 4.66 (td, J=13.4, 4.5 Hz, 2H), 4.47 (h, J=7.2 Hz, 1H), 3.63-3.41 (m, 4H), 2.46-2.28 (m, 2H), 2.05-2.01 (m, 2H), 1.90 (ddd, J=12.9, 8.2, 5.0 Hz, 1H), 1.79-1.64 (m, 4H). MS m/z: 468 [M+H]⁺.

### Example 195: N-[1-(2,2-difluoroethyl)-1H-pyrazolo[3,4-b]pyrazin-6-yl]-8-[6-(trifluoromethyl)pyridin-3-yl]-8-azaspiro[4.5]decan-2-amine

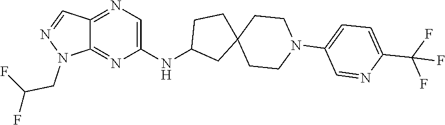 [SMILES: FC(F)Cn1ncc2ncc(NC3CCC4(CCN(c5ccc(C(F)(F)F)nc5)CC4)C3)nc21]

Followed the General Procedure A to afford the desired product as a colorless oil (17 mg, 53%). ¹H NMR (500 MHz, CDCl₃) δ 8.34 (d, J=2.8 Hz, 1H), 8.01 (s, 1H), 7.83 (s, 1H), 7.50 (d, J=8.8 Hz, 1H), 7.26 (s, 1H), 7.19 (dd, J=8.8, 2.9 Hz, 1H), 6.21 (tt, J=55.6, 4.5 Hz, 1H), 5.03 (d, J=6.6 Hz, 1H), 4.66 (td, J=13.3, 4.5 Hz, 2H), 4.41 (h, J=7.4 Hz, 1H), 3.42-3.24 (m, 4H), 2.39-2.29 (m, 1H), 2.26 (dd, J=13.2, 7.5 Hz, 1H), 1.82-1.70 (m, 5H), 1.69-1.63 (m, 2H), 1.43 (dd, J=13.2, 7.9 Hz, 1H). MS m/z: 483 [M+H]⁺.

### Example 196: N-[1-(2,2-difluoroethyl)-1H-pyrazolo[3,4-b]pyrazin-6-yl]-8-[4-(trifluoromethyl)pyridin-2-yl]-8-azaspiro[4.5]decan-2-amine

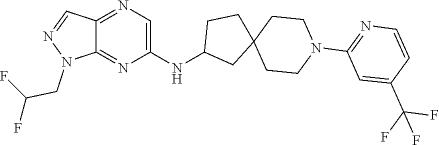 [SMILES: FC(F)Cn1ncc2ncc(NC3CCC4(CCN(c5cc(C(F)(F)F)ccn5)CC4)C3)nc21]

Followed the General Procedure A to afford the desired product as a colorless oil (18 mg, 56%). ¹H NMR (500 MHz, CDCl₃) δ 8.28 (d, J=5.1 Hz, 1H), 8.01 (s, 1H), 7.83 (s, 1H), 6.81 (d, J=1.5 Hz, 1H), 6.73 (dd, J=5.2, 1.3 Hz, 1H), 6.22 (tt, J=55.6, 4.5 Hz, 1H), 5.03 (d, J=6.6 Hz, 1H), 4.66 (td, J=13.4, 4.5 Hz, 2H), 4.41 (h, J=7.3 Hz, 1H), 3.68-3.53 (m, 4H), 2.38-2.29 (m, 1H), 2.26 (dd, J=13.2, 7.5 Hz, 1H), 1.80-1.58 (m, 8H), 1.43 (dd, J=13.2, 7.9 Hz, 1H). MS m/z: 483 [M+H]⁺.

### Example 197: (5r,8r)-N-[1-(2,2-difluoroethyl)-1H-pyrazolo[3,4-b]pyrazin-6-yl]-2-[4-(trifluoromethyl)pyridin-2-yl]-2-azaspiro[4.5]decan-8-amine

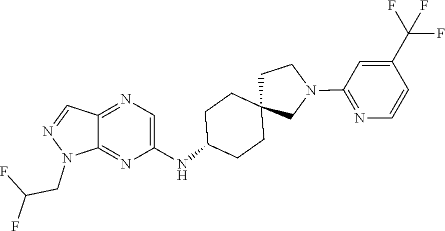 [SMILES: FC(F)Cn1ncc2ncc(N[C@H]3CC[C@@]4(CCN(c5cc(C(F)(F)F)ccn5)C4)CC3)nc21]

Followed the General Procedure A to afford the desired product as a white solid (14 mg, 43%). ¹H NMR (500 MHz, CDCl₃) δ 8.28 (d, J=5.2 Hz, 1H), 8.01 (s, 1H), 7.81 (s, 1H), 6.71 (dd, J=5.3, 1.5 Hz, 1H), 6.52 (s, 1H), 6.21 (tt, J=55.6, 4.5 Hz, 1H), 4.88 (d, J=7.5 Hz, 1H), 4.65 (td, J=13.3, 4.5 Hz, 2H), 3.97 (dq, J=10.8, 3.3 Hz, 1H), 3.54 (t, J=7.0 Hz, 2H), 3.46 (s, 2H), 2.13 (dt, J=12.8, 4.0 Hz, 2H), 1.93 (t, J=7.1 Hz, 2H), 1.84-1.75 (m, 2H), 1.63 (td, J=13.3, 3.7 Hz, 3H), 1.49-1.36 (m, 2H). MS m/z: 483 [M+H]⁺.

### Example 198: N-[1-(2,2-difluoroethyl)-1H-pyrazolo[3,4-b]pyrazin-6-yl]-2-[5-(trifluoromethyl)pyridin-2-yl]-2-azaspiro[3.5]nonan-7-amine

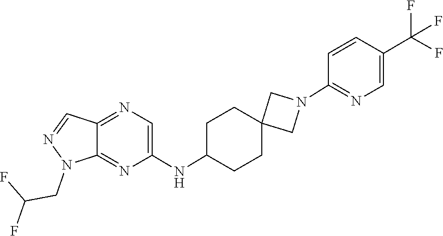 [SMILES: FC(F)Cn1ncc2ncc(NC3CCC4(CC3)CN(c3ccc(C(F)(F)F)cn3)C4)nc21]

Followed the General Procedure A to afford the desired product as a light-yellow oil (26 mg, 60%). ¹H NMR (500 MHz, CDCl₃) δ 8.36 (dt, J=2.0, 1.0 Hz, 1H), 8.01 (s, 1H), 7.82 (s, 1H), 7.60 (dd, J=8.8, 2.4 Hz, 1H), 6.28 (d, J=8.8 Hz, 1H), 6.21 (tt, J=55.6, 4.5 Hz, 1H), 4.84 (d, J=7.4 Hz, 1H), 4.65 (td, J=13.3, 4.5 Hz, 2H), 4.01-3.90 (m, 1H), 3.85 (s, 2H), 3.81 (s, 2H), 2.22-2.09 (m, 2H), 2.09-2.01 (m, 2H), 1.84-1.74 (m, 2H), 1.39 (t, J=12.1 Hz, 2H). MS m/z: 469 [M+H]⁺.

### Example 199: N-[1-(2,2-difluoroethyl)-1H-pyrazolo[3,4-b]pyrazin-6-yl]-2-[5-(trifluoromethyl)pyridin-3-yl]-2-azaspiro[3.5]nonan-7-amine

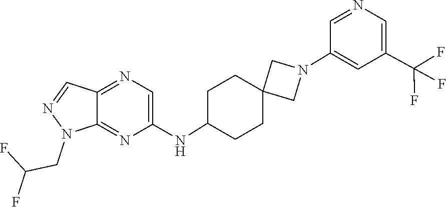 [SMILES: FC(F)Cn1ncc2ncc(NC3CCC4(CC3)CN(c3cncc(C(F)(F)F)c3)C4)nc21]

Followed the General Procedure A to afford the desired product as a light-yellow solid (14 mg, 39%). MS m/z: 469 [M+H]⁺.

### Example 200: N-[1-(2,2-difluoroethyl)-1H-pyrazolo[3,4-b]pyrazin-6-yl]-2-[6-(trifluoromethyl)pyridin-3-yl]-2-azaspiro[3.5]nonan-7-amine

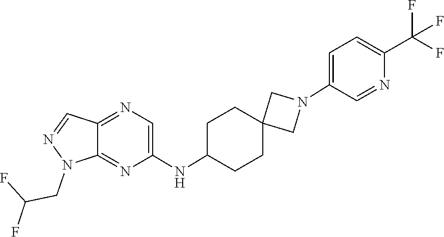 [SMILES: FC(F)Cn1ncc2ncc(NC3CCC4(CC3)CN(c3ccc(C(F)(F)F)nc3)C4)nc21]

Followed the General Procedure A to afford the desired product as a light-yellow oil (17 mg, 47%). ¹H NMR (500 MHz, CDCl₃) δ 8.02 (s, 1H), 7.87 (d, J=2.7 Hz, 1H), 7.82 (s, 1H), 7.47 (d, J=8.6 Hz, 1H), 6.72 (dd, J=8.6, 2.8 Hz, 1H), 6.22 (tt, J=55.6, 4.5 Hz, 1H), 4.82 (d, J=7.4 Hz, 2H), 4.66 (td, J=13.3, 4.5 Hz, 2H), 4.02-3.88 (m, 1H), 3.77 (s, 2H), 3.74 (s, 2H), 2.17-2.11 (m, 2H), 2.08 (d, J=13.5 Hz, 2H), 1.85-1.75 (m, 2H), 1.39 (q, J=12.0 Hz, 2H). MS m/z: 469 [M+H]⁺.

### Example 201: 7-[1-(2,2-difluoroethyl)-1H-pyrazolo[3,4-b]pyrazin-6-yl]-2-[6-(trifluoromethyl)pyridin-3-yl]-2,7-diazaspiro[4.4]nonan-1-one

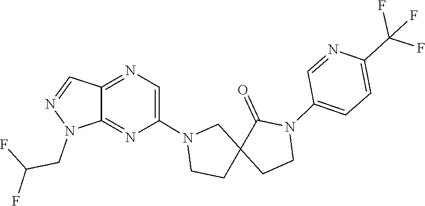 [SMILES: O=C1N(c2ccc(C(F)(F)F)nc2)CCC12CCN(c1cnc3cnn(CC(F)F)c3n1)C2]

Followed the General Procedure A to afford the desired product as a colorless oil (30 mg, 65%). ¹H NMR (500 MHz, CDCl₃) δ 8.88 (d, J=2.6 Hz, 1H), 8.53 (dd, J=8.7, 2.6 Hz, 1H), 8.06 (s, 1H), 7.99 (s, 1H), 7.73 (d, J=8.8 Hz, 1H), 6.22 (tt, J=55.6, 4.5 Hz, 1H), 4.66 (tdd, J=13.4, 4.5, 1.7 Hz, 2H), 4.07-3.93 (m, 4H), 3.83-3.68 (m, 2H), 2.63-2.50 (m, 1H), 2.43-2.32 (m, 2H), 2.18 (ddd, J=12.0, 7.2, 4.3 Hz, 1H). MS m/z: 469 [M+H]⁺.

### Example 202: 7-[1-(2,2-difluoroethyl)-1H-pyrazolo[3,4-b]pyrazin-6-yl]-2-[5-(trifluoromethyl)pyridin-2-yl]-2,7-diazaspiro[4.4]nonan-1-one

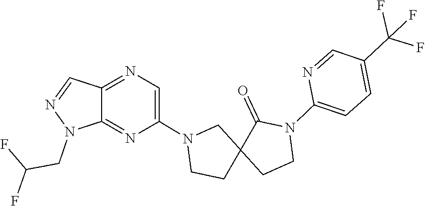 [SMILES: O=C1N(c2ccc(C(F)(F)F)cn2)CCC12CCN(c1cnc3cnn(CC(F)F)c3n1)C2]

Followed the General Procedure A to afford the desired product as a colorless oil (20 mg, 69%). ¹H NMR (500 MHz, CDCl₃) δ 8.65 (q, J=1.1 Hz, 1H), 8.58 (d, J=8.9 Hz, 1H), 8.06 (s, 1H), 7.99 (s, 1H), 7.94 (dd, J=8.9, 2.5 Hz, 1H), 6.22 (tt, J=55.6, 4.5 Hz, 1H), 4.66 (td, J=13.3, 4.5 Hz, 2H), 4.24-4.11 (m, 2H), 4.00 (t, J=8.7 Hz, 2H), 3.81-3.70 (m, 2H), 2.55 (dt, J=15.2, 8.0 Hz, 1H), 2.29 (tt, J=13.1, 7.3 Hz, 2H), 2.18 (ddd, J=12.3, 7.3, 4.5 Hz, 1H). MS m/z: 468 [M+H]⁺.

### Example 203: 7-[1-(2,2-difluoroethyl)-1H-pyrazolo[3,4-b]pyrazin-6-yl]-2-[6-(trifluoromethyl)pyridin-2-yl]-2,7-diazaspiro[4.4]nonan-1-one

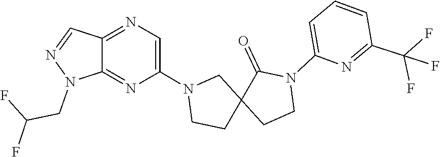 [SMILES: O=C1N(c2cccc(C(F)(F)F)n2)CCC12CCN(c1cnc3cnn(CC(F)F)c3n1)C2]

Followed the General Procedure A to afford the desired product as a colorless oil (33 mg, 65%). ¹H NMR (500 MHz, CDCl₃) δ 8.62 (d, J=8.5 Hz, 1H), 8.04 (s, 1H), 7.97 (s, 1H), 7.87 (t, J=8.0 Hz, 1H), 7.44 (d, J=7.3 Hz, 1H), 6.34-6.07 (m, 1H), 4.64 (td, J=13.3, 4.5 Hz, 2H), 4.24-4.12 (m, 2H), 4.01-3.92 (m, 2H), 3.79-3.69 (m, 2H), 2.57-2.48 (m, 1H), 2.32-2.21 (m, 2H), 2.20-2.12 (m, 1H). MS m/z: 468 [M+H]⁺.

### Example 204: 7-[1-(2,2-difluoroethyl)-1H-pyrazolo[3,4-b]pyrazin-6-yl]-2-[5-(trifluoromethyl)pyridin-3-yl]-2,7-diazaspiro[4.4]nonan-1-one

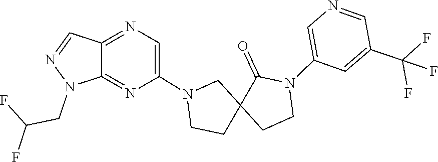 [SMILES: O=C1N(c2cncc(C(F)(F)F)c2)CCC12CCN(c1cnc3cnn(CC(F)F)c3n1)C2]

Followed the General Procedure A to afford the desired product as a white foam (16 mg, 50%). ¹H NMR (500 MHz, CDCl₃) δ 8.97 (d, J=2.5 Hz, 1H), 8.68 (s, 1H), 8.58 (t, J=2.0 Hz, 1H), 8.04 (s, 1H), 7.97 (s, 1H), 6.38-6.05 (m, 1H), 4.71-4.58 (m, 2H), 4.05-3.91 (m, 4H), 3.83-3.67 (m, 2H), 2.60-2.47 (m, 1H), 2.44-2.30 (m, 2H), 2.22-2.13 (m, 1H). MS m/z: 468 [M+H]⁺.

### Example 205: 9-[1-(2,2-difluoroethyl)-1H-pyrazolo[3,4-b]pyrazin-6-yl]-4-[6-(trifluoromethyl)pyridin-3-yl]-1-oxa-4,9-diazaspiro[5.5]undecane

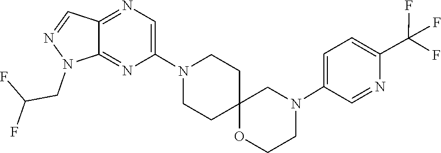 [SMILES: FC(F)Cn1ncc2ncc(N3CCC4(CC3)CN(c3ccc(C(F)(F)F)nc3)CCO4)nc21]

Followed the General Procedure A to afford the desired product as a colorless oil (27 mg, 75%). ¹H NMR (500 MHz, CDCl₃) δ 8.30 (d, J=2.9 Hz, 1H), 8.23 (s, 1H), 8.02 (s, 1H), 7.51 (d, J=8.8 Hz, 1H), 7.16 (dd, J=8.7, 2.9 Hz, 1H), 6.35-6.07 (m, 1H), 4.64 (td, J=13.3, 4.5 Hz, 2H), 4.24 (dt, J=14.1, 3.8 Hz, 2H), 3.98-3.89 (m, 2H), 3.43 (ddd, J=13.4, 11.9, 2.7 Hz, 2H), 3.34-3.29 (m, 2H), 3.13 (s, 2H), 2.17 (d, J=12.0 Hz, 2H), 1.63 (ddd, J=13.8, 12.1, 4.5 Hz, 2H). MS m/z: 484 [M+H]⁺.

### Example 206: 9-[1-(2,2-difluoroethyl)-1H-pyrazolo[3,4-b]pyrazin-6-yl]-4-[5-(trifluoromethyl)pyridin-3-yl]-1-oxa-4,9-diazaspiro[5.5]undecane

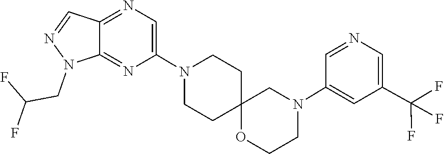 [SMILES: FC(F)Cn1ncc2ncc(N3CCC4(CC3)CN(c3cncc(C(F)(F)F)c3)CCO4)nc21]

Followed the General Procedure A to afford the desired product as a white solid (26 mg, 73%). ¹H NMR (500 MHz, CDCl₃) δ 8.42 (d, J=2.8 Hz, 1H), 8.35 (s, 1H), 8.24 (s, 1H), 8.02 (s, 1H), 7.27 (t, J=2.4 Hz, 1H), 6.34-6.06 (m, 1H), 4.64 (td, J=13.3, 4.5 Hz, 2H), 4.25 (dt, J=13.5, 3.8 Hz, 2H), 3.99-3.92 (m, 2H), 3.44 (ddd, J=13.3, 11.9, 2.7 Hz, 2H), 3.30-3.23 (m, 2H), 3.08 (s, 2H), 2.24-2.16 (m, 2H), 1.65 (ddd, J=13.8, 12.0, 4.5 Hz, 2H). MS m/z: 484 [M+H]⁺.

### Example 207: 9-[1-(2,2-difluoroethyl)-1H-pyrazolo[3,4-b]pyrazin-6-yl]-4-[5-(trifluoromethyl)pyridin-2-yl]-1-oxa-4,9-diazaspiro[5.5]undecane

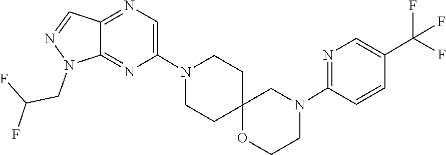 [SMILES: FC(F)Cn1ncc2ncc(N3CCC4(CC3)CN(c3ccc(C(F)(F)F)cn3)CCO4)nc21]

Followed the General Procedure A to afford the desired product as a light-yellow oil (40 mg, 80%). ¹H NMR (500 MHz, CDCl₃) δ 8.37-8.33 (m, 1H), 8.22 (s, 1H), 8.01 (s, 1H), 7.62 (dd, J=9.1, 2.5 Hz, 1H), 6.60 (d, J=9.0 Hz, 1H), 6.33-6.06 (m, 1H), 4.63 (td, J=13.4, 4.5 Hz, 2H), 4.14 (dt, J=13.6, 4.2 Hz, 2H), 3.87 (dd, J=5.9, 4.3 Hz, 2H), 3.67-3.62 (m, 2H), 3.59 (s, 2H), 3.49 (ddd, J=13.9, 11.3, 3.0 Hz, 2H), 2.03 (d, J=13.6 Hz, 2H), 1.65 (ddd, J=14.5, 11.4, 4.4 Hz, 2H). MS m/z: 484 [M+H]⁺.

### Example 208: 9-[1-(2,2-difluoroethyl)-1H-pyrazolo[3,4-b]pyrazin-6-yl]-4-[6-(trifluoromethyl)pyridin-2-yl]-1-oxa-4,9-diazaspiro[5.5]undecane

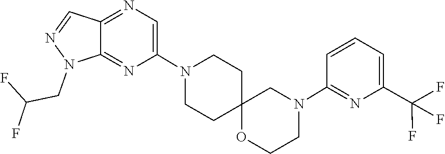 [SMILES: FC(F)Cn1ncc2ncc(N3CCC4(CC3)CN(c3cccc(C(F)(F)F)n3)CCO4)nc21]

Followed the General Procedure A to afford the desired product as a white foam (23 mg, 64%). ¹H NMR (500 MHz, CDCl₃) δ 8.22 (s, 1H), 8.01 (s, 1H), 7.57 (t, J=8.0 Hz, 1H), 6.94 (d, J=7.2 Hz, 1H), 6.73 (d, J=8.7 Hz, 1H), 6.32-6.06 (m, 1H), 4.63 (td, J=13.3, 4.5 Hz, 2H), 4.13 (dt, J=13.3, 3.9 Hz, 2H), 3.90-3.83 (m, 2H), 3.65-3.59 (m, 2H), 3.54 (s, 2H), 3.51 (ddd, J=13.8, 11.2, 3.1 Hz, 2H), 2.04 (d, J=13.6 Hz, 2H), 1.66 (ddd, J=14.6, 11.1, 4.4 Hz, 2H). MS m/z: 484 [M+H]⁺.

### Example 209: 4-[1-(2,2-difluoroethyl)-1H-pyrazolo[3,4-b]pyrazin-6-yl]-9-[6-(trifluoromethyl)pyridin-3-yl]-1-oxa-4,9-diazaspiro[5.5]undecane

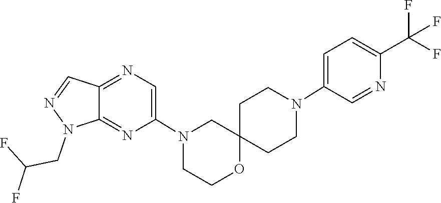 [SMILES: FC(F)Cn1ncc2ncc(N3CCOC4(CCN(c5ccc(C(F)(F)F)nc5)CC4)C3)nc21]

Followed the General Procedure A to afford the desired product as a white foam (28 mg, 70%). ¹H NMR (500 MHz, CDCl₃) δ 8.34 (d, J=2.9 Hz, 1H), 8.17 (s, 1H), 8.04 (s, 1H), 7.49 (d, J=8.8 Hz, 1H), 7.19 (dd, J=8.8, 2.9 Hz, 1H), 6.32-6.04 (m, 1H), 4.63 (td, J=13.4, 4.4 Hz, 2H), 3.89 (dd, J=6.1, 4.1 Hz, 2H), 3.77 (dd, J=6.0, 4.1 Hz, 2H), 3.66 (s, 2H), 3.55 (dt, J=12.8, 4.1 Hz, 2H), 3.27 (td, J=12.3, 2.9 Hz, 2H), 2.05 (d, J=13.8 Hz, 2H), 1.80-1.68 (m, 2H). MS m/z: 484 [M+H]⁺.

### Example 210: 4-[1-(2,2-difluoroethyl)-1H-pyrazolo[3,4-b]pyrazin-6-yl]-9-[5-(trifluoromethyl)pyridin-3-yl]-1-oxa-4,9-diazaspiro[5.5]undecane

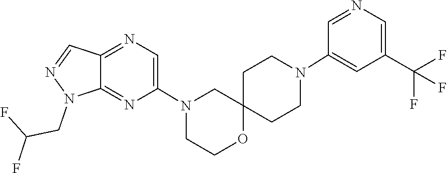 [SMILES: FC(F)Cn1ncc2ncc(N3CCOC4(CCN(c5cncc(C(F)(F)F)c5)CC4)C3)nc21]

Followed the General Procedure A to afford the desired product as a light-yellow oil (26 mg, 70%). ¹H NMR (500 MHz, CDCl₃) δ 8.46 (d, J=2.8 Hz, 1H), 8.29 (d, J=1.6 Hz, 1H), 8.18 (s, 1H), 8.05 (s, 1H), 7.31 (t, J=2.5 Hz, 1H), 6.33-6.05 (m, 1H), 4.63 (td, J=13.4, 4.5 Hz, 2H), 3.89 (dd, J=5.9, 4.1 Hz, 2H), 3.77 (dd, J=6.0, 4.1 Hz, 2H), 3.67 (s, 2H), 3.53-3.46 (m, 2H), 3.23 (td, J=12.0, 2.9 Hz, 2H), 2.07 (d, J=13.7 Hz, 2H), 1.80-1.69 (m, 2H). MS m/z: 484 [M+H]⁺.

### Example 211: 4-[1-(2,2-difluoroethyl)-1H-pyrazolo[3,4-b]pyrazin-6-yl]-9-[6-(trifluoromethyl)pyridin-2-yl]-1-oxa-4,9-diazaspiro[5.5]undecane

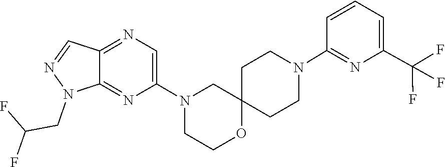 [SMILES: FC(F)Cn1ncc2ncc(N3CCOC4(CCN(c5cccc(C(F)(F)F)n5)CC4)C3)nc21]

Followed the General Procedure A to afford the desired product as a colorless oil (38 mg, 76%). ¹H NMR (500 MHz, CDCl₃) δ 8.16 (s, 1H), 8.03 (s, 1H), 7.55 (t, J=8.0 Hz, 1H), 6.90 (d, J=7.2 Hz, 1H), 6.78 (d, J=8.7 Hz, 1H), 6.30-6.04 (m, 1H), 4.62 (td, J=13.4, 4.4 Hz, 2H), 4.00 (dt, J=13.4, 4.2 Hz, 2H), 3.89 (dd, J=5.9, 4.3 Hz, 2H), 3.77 (dd, J=6.0, 4.1 Hz, 2H), 3.64 (s, 2H), 3.38 (ddd, J=13.6, 11.1, 3.0 Hz, 2H), 1.98 (d, J=13.2 Hz, 2H), 1.68-1.60 (m, 2H). MS m/z: 484 [M+H]⁺.

### Example 212: 4-[1-(2,2-difluoroethyl)-1H-pyrazolo[3,4-b]pyrazin-6-yl]-9-[5-(trifluoromethyl)pyridin-2-yl]-1-oxa-4,9-diazaspiro[5.5]undecane

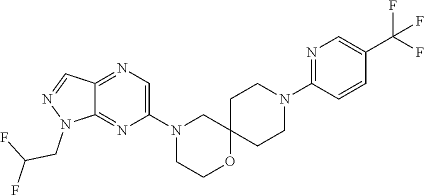 [SMILES: FC(F)Cn1ncc2ncc(N3CCOC4(CCN(c5ccc(C(F)(F)F)cn5)CC4)C3)nc21]

Followed the General Procedure A to afford the desired product as a colorless oil (37 mg, 86%). ¹H NMR (500 MHz, CDCl₃) δ 8.39-8.35 (m, 1H), 8.16 (s, 1H), 8.04 (s, 1H), 7.60 (dd, J=9.1, 2.5 Hz, 1H), 6.65 (d, J=9.0 Hz, 1H), 6.31-6.04 (m, 1H), 4.62 (td, J=13.4, 4.5 Hz, 2H), 4.06 (dt, J=13.5, 3.9 Hz, 2H), 3.94-3.85 (m, 2H), 3.77 (dd, J=6.1, 4.0 Hz, 2H), 3.64 (s, 2H), 3.39 (ddd, J=13.8, 11.4, 2.9 Hz, 2H), 2.00 (d, J=13.5 Hz, 2H), 1.68-1.57 (m, 2H). MS m/z: 484 [M+H]⁺.

### Example 213: 9-[1-(2,2-difluoroethyl)-1H-pyrazolo[3,4-b]pyrazin-6-yl]-2-[5-(trifluoromethyl)pyridin-2-yl]-2,9-diazaspiro[5.5]undecane

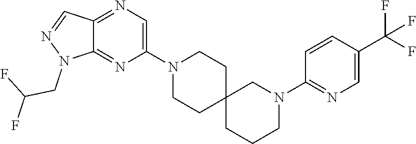 [SMILES: FC(F)Cn1ncc2ncc(N3CCC4(CCCN(c5ccc(C(F)(F)F)cn5)C4)CC3)nc21]

Followed the General Procedure A to afford the desired product as a colorless oil (28 mg, 65%). ¹H NMR (500 MHz, CDCl₃) δ 8.34 (dt, J=2.7, 0.9 Hz, 1H), 8.21 (s, 1H), 8.02 (s, 1H), 7.59 (dd, J=9.1, 2.6 Hz, 1H), 6.63 (d, J=9.1 Hz, 1H), 6.21 (tt, J=55.6, 4.5 Hz, 1H), 4.64 (td, J=13.4, 4.5 Hz, 2H), 3.91 (ddd, J=13.7, 6.4, 4.2 Hz, 2H), 3.75 (s, 2H), 3.68 (ddd, J=13.3, 9.2, 3.6 Hz, 2H), 3.59 (t, J=5.6 Hz, 2H), 1.77-1.59 (m, 6H), 1.54 (td, J=9.3, 4.6 Hz, 2H). MS m/z: 483 [M+H]⁺.

### Example 214: 9-[1-(2,2-difluoroethyl)-1H-pyrazolo[3,4-b]pyrazin-6-yl]-2-[6-(trifluoromethyl)pyridin-3-yl]-2,9-diazaspiro[5.5]undecane

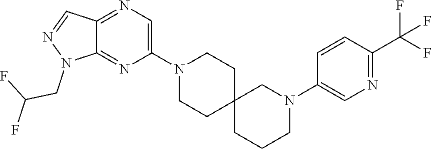 [SMILES: FC(F)Cn1ncc2ncc(N3CCC4(CCCN(c5ccc(C(F)(F)F)nc5)C4)CC3)nc21]

Followed the General Procedure A to afford the desired product as a colorless oil (28 mg, 65%). ¹H NMR (500 MHz, CDCl₃) δ 8.32 (d, J=2.9 Hz, 1H), 8.20 (s, 1H), 8.02 (s, 1H), 7.47 (d, J=8.7 Hz, 1H), 7.16 (dd, J=8.7, 2.9 Hz, 1H), 6.34-6.05 (m, 1H), 4.64 (td, J=13.4, 4.5 Hz, 2H), 3.78 (dt, J=13.6, 5.7 Hz, 2H), 3.70 (dt, J=13.7, 5.9 Hz, 2H), 3.30 (t, J=5.7 Hz, 2H), 3.18 (s, 2H), 1.85-1.74 (m, 2H), 1.68 (t, J=5.9 Hz, 4H), 1.64 (dd, J=7.8, 5.0 Hz, 2H). MS m/z: 483 [M+H]⁺.

### Example 215: 9-[1-(2,2-difluoroethyl)-1H-pyrazolo[3,4-b]pyrazin-6-yl]-2-[5-(trifluoromethyl)pyridin-3-yl]-2,9-diazaspiro[5.5]undecane

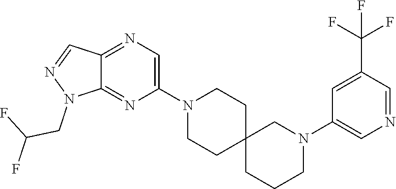 [SMILES: FC(F)Cn1ncc2ncc(N3CCC4(CCCN(c5cncc(C(F)(F)F)c5)C4)CC3)nc21]

Followed the General Procedure A to afford the desired product as a colorless oil (27 mg, 63%). ¹H NMR (500 MHz, CDCl₃) δ 8.46 (d, J=2.8 Hz, 1H), 8.30 (d, J=1.7 Hz, 1H), 8.23 (s, 1H), 8.04 (s, 1H), 7.30 (d, J=2.5 Hz, 1H), 6.22 (tt, J=55.6, 4.5 Hz, 1H), 4.66 (td, J=13.4, 4.5 Hz, 2H), 3.82 (dt, J=13.7, 5.7 Hz, 2H), 3.73 (dt, J=13.8, 6.0 Hz, 2H), 3.26 (t, J=5.6 Hz, 2H), 3.13 (s, 2H), 1.83 (q, J=5.8 Hz, 2H), 1.73 (t, J=5.9 Hz, 4H), 1.68-1.61 (m, 2H). MS m/z: 483 [M+H]⁺.

### Example 216: 9-[1-(2,2-difluoroethyl)-1H-pyrazolo[3,4-b]pyrazin-6-yl]-2-[6-(trifluoromethyl)pyridin-2-yl]-2,9-diazaspiro[5.5]undecane

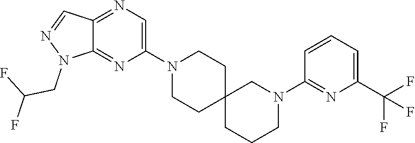 [SMILES: FC(F)Cn1ncc2ncc(N3CCC4(CCCN(c5cccc(C(F)(F)F)n5)C4)CC3)nc21]

Followed the General Procedure A to afford the desired product as a colorless oil (23 mg, 54%). ¹H NMR (500 MHz, CDCl₃) δ 8.18 (s, 1H), 8.00 (s, 1H), 7.51 (t, J=8.0 Hz, 1H), 6.84 (d, J=7.2 Hz, 1H), 6.74 (d, J=8.7 Hz, 1H), 6.36-6.05 (m, 1H), 4.62 (td, J=13.3, 4.5 Hz, 2H), 3.95 (dt, J=13.7, 5.0 Hz, 2H), 3.73 (s, 2H), 3.63 (ddd, J=13.5, 9.8, 3.4 Hz, 2H), 3.55 (t, J=5.6 Hz, 2H), 1.73-1.61 (m, 4H), 1.61-1.55 (m, 2H), 1.49 (ddd, J=13.8, 9.8, 4.2 Hz, 2H). MS m/z: 483 [M+H]⁺.

### Example 217: 2-[1-(2,2-difluoroethyl)-1H-pyrazolo[3,4-b]pyrazin-6-yl]-9-[4-(trifluoromethyl)pyridin-2-yl]-2,9-diazaspiro[5.5]undecane

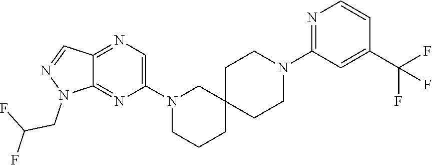 [SMILES: FC(F)Cn1ncc2ncc(N3CCCC4(CCN(c5cc(C(F)(F)F)ccn5)CC4)C3)nc21]

Followed the General Procedure A to afford the desired product as a colorless oil (33 mg, 64%). ¹H NMR (500 MHz, CDCl₃) δ 8.27 (d, J=5.0 Hz, 1H), 8.20 (s, 1H), 8.00 (s, 1H), 6.78 (s, 1H), 6.73 (d, J=5.2 Hz, 1H), 6.31-6.02 (m, 1H), 4.60 (td, J=13.4, 4.4 Hz, 2H), 3.76-3.64 (m, 6H), 3.59 (ddd, J=13.1, 8.4, 3.9 Hz, 2H), 1.79-1.71 (m, 2H), 1.68-1.59 (m, 4H), 1.59-1.50 (m, 2H). MS m/z: 483 [M+H]⁺.

### Example 218: 2-[1-(2,2-difluoroethyl)-1H-pyrazolo[3,4-b]pyrazin-6-yl]-9-[5-(trifluoromethyl)pyridin-2-yl]-2,9-diazaspiro[5.5]undecane

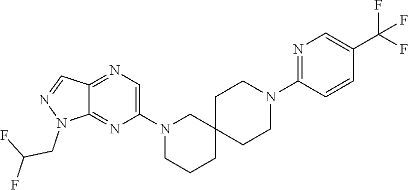 [SMILES: FC(F)Cn1ncc2ncc(N3CCCC4(CCN(c5ccc(C(F)(F)F)cn5)CC4)C3)nc21]

Followed the General Procedure A to afford the desired product as a colorless oil (37 mg, 66%). ¹H NMR (500 MHz, CDCl₃) δ 8.38 (dt, J=2.8, 0.9 Hz, 1H), 8.21 (s, 1H), 8.02 (s, 1H), 7.61 (dd, J=9.1, 2.6 Hz, 1H), 6.63 (d, J=9.0 Hz, 1H), 6.19 (tt, J=55.6, 4.4 Hz, 1H), 4.62 (td, J=13.4, 4.4 Hz, 2H), 3.74 (q, J=8.8, 7.2 Hz, 6H), 3.64 (ddd, J=13.2, 8.5, 3.8 Hz, 2H), 1.76 (q, J=7.0, 5.9 Hz, 2H), 1.64 (ddd, J=20.2, 7.0, 4.0 Hz, 4H), 1.54 (dd, J=13.6, 4.5 Hz, 2H). MS m/z: 483 [M+H]⁺.

### Example 219: 2-[1-(2,2-difluoroethyl)-1H-pyrazolo[3,4-b]pyrazin-6-yl]-9-[6-(trifluoromethyl)pyridin-2-yl]-2,9-diazaspiro[5.5]undecane

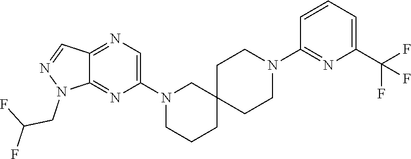 [SMILES: FC(F)Cn1ncc2ncc(N3CCCC4(CCN(c5cccc(C(F)(F)F)n5)CC4)C3)nc21]

Followed the General Procedure A to afford the desired product as a colorless oil (35 mg, 64%). ¹H NMR (500 MHz, CDCl₃) δ 8.19 (s, 1H), 8.00 (s, 1H), 7.54 (t, J=8.0 Hz, 1H), 6.90 (d, J=7.2 Hz, 1H), 6.76 (d, J=8.7 Hz, 1H), 6.29-6.03 (m, 1H), 4.60 (td, J=13.4, 4.4 Hz, 2H), 3.79-3.64 (m, 6H), 3.58 (ddd, J=13.1, 8.6, 3.8 Hz, 2H), 1.78-1.70 (m, 2H), 1.67-1.59 (m, 4H), 1.58-1.48 (m, 2H). MS m/z: 483 [M+H]⁺.

### Example 220: 2-[1-(2,2-difluoroethyl)-1H-pyrazolo[3,4-b]pyrazin-6-yl]-9-[6-(trifluoromethyl)pyridin-3-yl]-2,9-diazaspiro[5.5]undecane

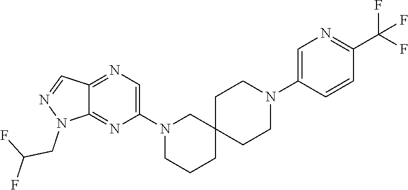 [SMILES: FC(F)Cn1ncc2ncc(N3CCCC4(CCN(c5ccc(C(F)(F)F)nc5)CC4)C3)nc21]

Followed the General Procedure A to afford the desired product as a colorless oil (28 mg, 61%). ¹H NMR (500 MHz, CDCl₃) δ 8.32 (d, J=2.9 Hz, 1H), 8.20 (s, 1H), 8.00 (s, 1H), 7.49 (d, J=8.8 Hz, 1H), 7.17 (dd, J=8.7, 2.9 Hz, 1H), 6.30-6.03 (m, 1H), 4.59 (td, J=13.4, 4.4 Hz, 2H), 3.76-3.70 (m, 4H), 3.43-3.31 (m, 4H), 1.79-1.57 (m, 8H). MS m/z: 483 [M+H]⁺.

### Example 221: 2-[1-(2,2-difluoroethyl)-1H-pyrazolo[3,4-b]pyrazin-6-yl]-9-[5-(trifluoromethyl)pyridin-3-yl]-2,9-diazaspiro[5.5]undecane

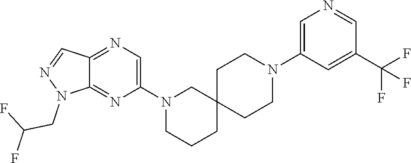 [SMILES: FC(F)Cn1ncc2ncc(N3CCCC4(CCN(c5cncc(C(F)(F)F)c5)CC4)C3)nc21]

Followed the General Procedure A to afford the desired product as a colorless oil (19 mg, 44%). ¹H NMR (500 MHz, CDCl₃) δ 8.44 (d, J=2.8 Hz, 1H), 8.28 (s, 1H), 8.20 (s, 1H), 8.00 (s, 1H), 7.30 (t, J=2.5 Hz, 1H), 6.29-6.01 (m, 1H), 4.59 (td, J=13.4, 4.4 Hz, 2H), 3.78-3.68 (m, 4H), 3.39-3.27 (m, 4H), 1.80-1.68 (m, 4H), 1.68-1.59 (m, 4H). MS m/z: 483 [M+H]⁺.

### Example 222: 2-[1-(2,2-difluoroethyl)-1H-pyrazolo[3,4-b]pyrazin-6-yl]-6-[2-(trifluoromethyl)pyridin-4-yl]-2,6-diazaspiro[3.4]octane

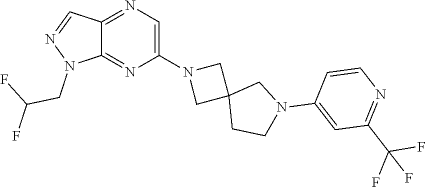 [SMILES: FC(F)Cn1ncc2ncc(N3CC4(CCN(c5ccnc(C(F)(F)F)c5)C4)C3)nc21]

Followed the General Procedure A to afford the desired product as a colorless oil (29 mg, 65%). ¹H NMR (500 MHz, CDCl₃) δ 8.33 (d, J=5.8 Hz, 1H), 8.08 (s, 1H), 7.82 (s, 1H), 6.77 (d, J=2.4 Hz, 1H), 6.51 (dd, J=5.8, 2.4 Hz, 1H), 6.22 (tt, J=55.6, 4.5 Hz, 1H), 4.66 (td, J=13.4, 4.5 Hz, 2H), 4.28-4.19 (m, 3H), 3.68 (s, 2H), 3.53 (t, J=6.9 Hz, 2H), 2.41 (t, J=6.9 Hz, 2H). MS m/z: 440 [M+H]⁺.

### Example 223: 6-[1-(2,2-difluoroethyl)-1H-pyrazolo[3,4-b]pyrazin-6-yl]-2-[2-(trifluoromethyl)pyridin-4-yl]-2,6-diazaspiro[3.4]octane

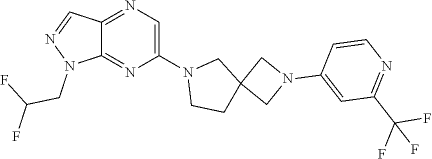 [SMILES: FC(F)Cn1ncc2ncc(N3CCC4(CN(c5ccnc(C(F)(F)F)c5)C4)C3)nc21]

Followed the General Procedure A to afford the desired product as a colorless oil (32 mg, 71%). ¹H NMR (500 MHz, CDCl₃) δ 8.34 (d, J=5.6 Hz, 1H), 8.07 (s, 1H), 7.97 (s, 1H), 6.64 (d, J=2.3 Hz, 1H), 6.39 (dd, J=5.6, 2.3 Hz, 1H), 6.22 (tt, J=55.6, 4.5 Hz, 2H), 4.67 (td, J=13.3, 4.5 Hz, 2H), 4.09 (d, J=8.0 Hz, 2H), 4.04 (d, J=7.9 Hz, 2H), 3.91 (s, 2H), 3.76 (t, J=6.9 Hz, 2H), 2.41 (t, J=6.9 Hz, 2H). MS m/z: 440 [M+H]⁺.

### Example 224: 9-[1-(2,2-difluoroethyl)-1H-pyrazolo[3,4-b]pyrazin-6-yl]-2-[4-(trifluoromethyl)pyridin-2-yl]-2,9-diazaspiro[5.5]undecane

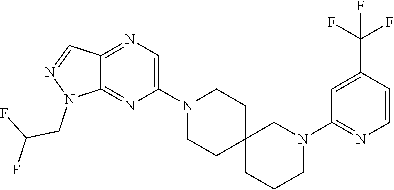 [SMILES: FC(F)Cn1ncc2ncc(N3CCC4(CCCN(c5cc(C(F)(F)F)ccn5)C4)CC3)nc21]

Followed the General Procedure A to afford the desired product as a colorless oil (32 mg, 65%). ¹H NMR (500 MHz, CDCl₃) δ 8.22 (d, J=5.1 Hz, 1H), 8.19 (s, 1H), 8.00 (s, 1H), 6.76 (s, 1H), 6.68 (d, J=3.9 Hz, 1H), 6.32-6.04 (m, 1H), 4.62 (td, J=13.3, 4.5 Hz, 2H), 3.92-3.82 (m, 2H), 3.72-3.63 (m, 4H), 3.53 (t, J=5.6 Hz, 2H), 1.76-1.63 (m, 4H), 1.61 (dd, J=7.6, 4.6 Hz, 2H), 1.56-1.49 (m, 2H). MS m/z: 483 [M+H]⁺.

### Example 225: 9-[1-(2,2-difluoroethyl)-1H-pyrazolo[3,4-b]pyrazin-6-yl]-2-[2-(trifluoromethyl)pyridin-4-yl]-2,9-diazaspiro[5.5]undecane

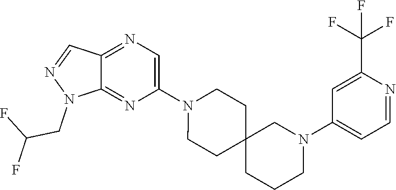 [SMILES: FC(F)Cn1ncc2ncc(N3CCC4(CCCN(c5ccnc(C(F)(F)F)c5)C4)CC3)nc21]

Followed the General Procedure A to afford the desired product as a colorless oil (22 mg, 51%). ¹H NMR (500 MHz, CDCl₃) δ 8.31 (d, J=6.0 Hz, 1H), 8.22 (s, 1H), 8.04 (s, 1H), 6.99 (d, J=2.6 Hz, 1H), 6.75 (dd, J=6.0, 2.7 Hz, 1H), 6.21 (tt, J=55.6, 4.5 Hz, 1H), 4.65 (td, J=13.4, 4.5 Hz, 2H), 3.84 (dt, J=13.7, 5.2 Hz, 2H), 3.68 (ddd, J=13.7, 7.3, 5.0 Hz, 2H), 3.44 (t, J=5.6 Hz, 2H), 3.31 (s, 2H), 1.82-1.75 (m, 2H), 1.75-1.68 (m, 2H), 1.67-1.64 (m, 2H), 1.62-1.53 (m, 2H). MS m/z: 483 [M+H]⁺.

### Example 226: 2-[1-(2,2-difluoroethyl)-1H-pyrazolo[3,4-b]pyrazin-6-yl]-9-[2-(trifluoromethyl)pyridin-4-yl]-2,9-diazaspiro[5.5]undecane

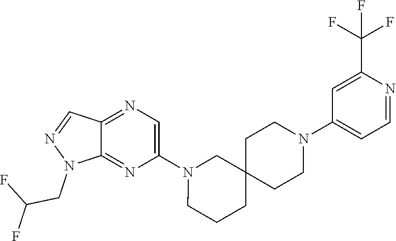 [SMILES: FC(F)Cn1ncc2ncc(N3CCCC4(CCN(c5ccnc(C(F)(F)F)c5)CC4)C3)nc21]

Followed the General Procedure A to afford the desired product as a colorless oil (4.4 mg, 10%). ¹H NMR (500 MHz, CDCl₃) δ 8.33 (d, J=5.9 Hz, 1H), 8.22 (s, 1H), 8.03 (s, 1H), 7.01 (d, J=2.6 Hz, 1H), 6.74 (dd, J=6.0, 2.6 Hz, 1H), 6.18 (tt, J=55.6, 4.4 Hz, 1H), 4.61 (td, J=13.4, 4.4 Hz, 2H), 3.75 (d, J=6.5 Hz, 4H), 3.57-3.40 (m, 4H), 1.81-1.73 (m, 2H), 1.73-1.65 (m, 2H), 1.59 (ddt, J=13.4, 8.3, 5.3 Hz, 4H). MS m/z: 483 [M+H]⁺.

### Example 227: 7-[1-(2,2-difluoroethyl)-1H-pyrazolo[3,4-b]pyrazin-6-yl]-N-[5-(trifluoromethyl)pyridin-2-yl]-7-azaspiro[3.5]nonan-2-amine

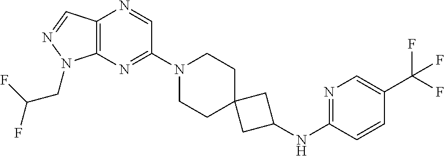 [SMILES: FC(F)Cn1ncc2ncc(N3CCC4(CC3)CC(Nc3ccc(C(F)(F)F)cn3)C4)nc21]

Followed the General Procedure A to afford the desired product as a colorless oil (10 mg, 31%). ¹H NMR (500 MHz, CDCl₃) δ 8.34 (dt, J=2.1, 1.0 Hz, 1H), 8.23 (s, 1H), 8.02 (s, 1H), 7.60 (dd, J=8.8, 2.4 Hz, 1H), 6.34 (d, J=8.8 Hz, 1H), 6.32 (tt, J=55.7, 4.5 Hz, 1H), 5.07 (d, J=6.3 Hz, 1H), 4.65 (td, J=13.4, 4.5 Hz, 2H), 4.28 (q, J=7.5 Hz, 1H), 3.78-3.72 (m, 2H), 3.70-3.64 (m, 2H), 2.52 (ddt, J=9.2, 7.9, 2.0 Hz, 2H), 1.84-1.70 (m, 6H). MS m/z: 468 [M+H]⁺.

### Example 228: 7-[1-(2,2-difluoroethyl)-1H-pyrazolo[3,4-b]pyrazin-6-yl]-N-[5-(trifluoromethyl)pyridin-3-yl]-7-azaspiro[3.5]nonan-2-amine

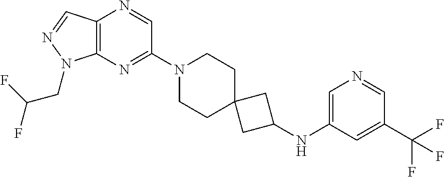 [SMILES: FC(F)Cn1ncc2ncc(N3CCC4(CC3)CC(Nc3cncc(C(F)(F)F)c3)C4)nc21]

Followed the General Procedure A to afford the desired product as a colorless oil (3.75 mg, 18%). ¹H NMR (500 MHz, CDCl₃) δ 8.23 (s, 1H), 8.22 (dd, J=2.0, 0.9 Hz, 1H), 8.12 (d, J=2.7 Hz, 1H), 8.03 (s, 1H), 6.92 (t, J=2.4 Hz, 1H), 6.22 (tt, J=55.6, 4.5 Hz, 1H), 4.66 (td, J=13.3, 4.5 Hz, 2H), 4.16 (d, J=5.6 Hz, 1H), 3.99 (pd, J=7.5, 5.5 Hz, 1H), 3.82-3.73 (m, 2H), 3.73-3.63 (m, 2H), 2.59-2.48 (m, 2H), 1.88-1.79 (m, 2H), 1.79-1.66 (m, 4H). MS m/z: 468 [M+H]⁺.

### Example 229: 7-[1-(2,2-difluoroethyl)-1H-pyrazolo[3,4-b]pyrazin-6-yl]-N-[2-(trifluoromethyl)pyridin-4-yl]-7-azaspiro[3.5]nonan-2-amine

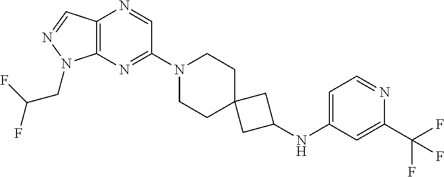 [SMILES: FC(F)Cn1ncc2ncc(N3CCC4(CC3)CC(Nc3ccnc(C(F)(F)F)c3)C4)nc21]

Followed the General Procedure A to afford the desired product as a colorless oil (21 mg, 40%). ¹H NMR (500 MHz, CDCl₃) δ 8.29 (d, J=5.7 Hz, 1H), 8.23 (s, 1H), 8.03 (s, 1H), 6.74 (d, J=2.3 Hz, 1H), 6.49 (dd, J=5.7, 2.3 Hz, 1H), 6.22 (tt, J=55.6, 4.5 Hz, 1H), 4.71-4.58 (m, 3H), 4.05 (td, J=7.6, 5.6 Hz, 1H), 3.79-3.73 (m, 2H), 3.71-3.65 (m, 2H), 2.58-2.50 (m, 2H), 1.85-1.71 (m, 6H). MS m/z: 468 [M+H]⁺.

### Example 230: 7-[1-(2,2-difluoroethyl)-1H-pyrazolo[3,4-b]pyrazin-6-yl]-N-[6-(trifluoromethyl)pyridin-3-yl]-7-azaspiro[3.5]nonan-2-amine

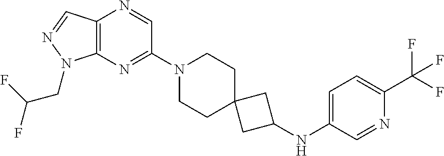 [SMILES: FC(F)Cn1ncc2ncc(N3CCC4(CC3)CC(Nc3ccc(C(F)(F)F)nc3)C4)nc21]

Followed the General Procedure A to afford the desired product as a white solid (24 mg, 55%). ¹H NMR (500 MHz, CDCl₃) δ 8.23 (s, 1H), 8.03 (s, 1H), 8.02 (d, J=2.8 Hz, 1H), 7.46 (d, J=8.6 Hz, 1H), 6.82 (dd, J=8.6, 2.8 Hz, 1H), 6.22 (tt, J=55.6, 4.5 Hz, 1H), 4.65 (td, J=13.4, 4.5 Hz, 2H), 4.28 (d, J=5.5 Hz, 1H), 4.01 (td, J=7.6, 5.6 Hz, 1H), 3.79-3.73 (m, 2H), 3.71-3.65 (m, 2H), 2.56-2.48 (m, 2H), 1.85-1.79 (m, 2H), 1.79-1.72 (m, 4H). MS m/z: 468 [M+H]⁺.

### Example 231: 7-[1-(2,2-difluoroethyl)-1H-pyrazolo[3,4-b]pyrazin-6-yl]-N-[4-(trifluoromethyl)pyridin-2-yl]-7-azaspiro[3.5]nonan-2-amine

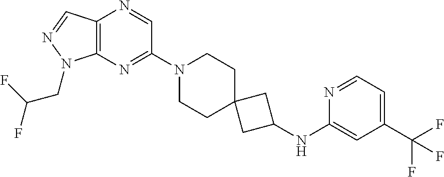 [SMILES: FC(F)Cn1ncc2ncc(N3CCC4(CC3)CC(Nc3cc(C(F)(F)F)ccn3)C4)nc21]

Followed the General Procedure A to afford the desired product as a colorless oil (5.6 mg, 35%). ¹H NMR (500 MHz, CDCl₃) δ 8.23 (s, 1H), 8.22 (s, 1H), 8.02 (s, 1H), 6.77 (dd, J=5.3, 1.5 Hz, 1H), 6.50-6.46 (m, 1H), 6.22 (tt, J=55.6, 4.5 Hz, 1H), 4.95 (d, J=6.4 Hz, 1H), 4.65 (td, J=13.4, 4.5 Hz, 2H), 4.31-4.20 (m, 1H), 3.78-3.72 (m, 2H), 3.70-3.64 (m, 2H), 2.57-2.49 (m, 2H), 1.85-1.79 (m, 2H), 1.79-1.71 (m, 4H). MS m/z: 468 [M+H]⁺.

### Example 232: 7-[1-(2,2-difluoroethyl)-1H-pyrazolo[3,4-b]pyrazin-6-yl]-N-[6-(trifluoromethyl)pyridin-2-yl]-7-azaspiro[3.5]nonan-2-amine

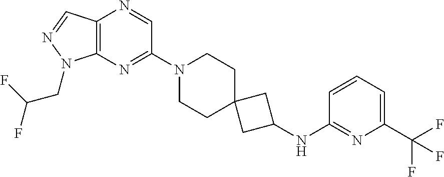 [SMILES: FC(F)Cn1ncc2ncc(N3CCC4(CC3)CC(Nc3cccc(C(F)(F)F)n3)C4)nc21]

Followed the General Procedure A to afford the desired product as a colorless oil (27 mg, 53%). ¹H NMR (500 MHz, CDCl₃) δ 8.23 (s, 1H), 8.02 (s, 1H), 7.55 (t, J=7.9 Hz, 1H), 6.95 (d, J=7.3 Hz, 1H), 6.46 (d, J=8.5 Hz, 1H), 6.22 (tt, J=55.6, 4.5 Hz, 2H), 4.98 (d, J=6.2 Hz, 1H), 4.65 (td, J=13.4, 4.5 Hz, 2H), 4.20 (h, J=7.5 Hz, 1H), 3.80-3.71 (m, 2H), 3.70-3.61 (m, 2H), 2.55-2.45 (m, 2H), 1.83-1.69 (m, 6H). MS m/z: 468 [M+H]⁺.

### Example 233: 8-[6-(1,3,4-thiadiazol-2-yl)pyrazin-2-yl]-2-[6-(trifluoromethyl)pyridin-3-yl]-2,8-diazaspiro[4.5]decane

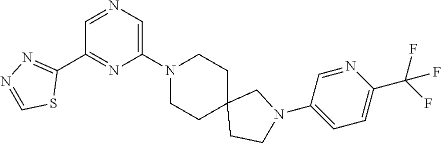 [SMILES: FC(F)(F)c1ccc(N2CCC3(CCN(c4cncc(-c5nncs5)n4)CC3)C2)cn1]

Followed the General Procedure A to afford the desired product as a yellow solid (25 mg, 74%). ¹H NMR (500 MHz, CDCl₃) δ 9.19 (s, 1H), 8.86 (d, J=0.5 Hz, 1H), 8.29 (d, J=0.6 Hz, 1H), 8.02 (d, J=2.8 Hz, 1H), 7.49 (d, J=8.7 Hz, 1H), 6.83 (dd, J=8.6, 2.9 Hz, 1H), 3.79 (ddd, J=13.6, 6.9, 4.5 Hz, 2H), 3.70 (ddd, J=13.6, 7.2, 4.5 Hz, 2H), 3.51 (t, J=7.0 Hz, 2H), 3.32 (s, 2H), 2.05 (t, J=7.0 Hz, 2H), 1.86-1.73 (m, 4H). MS m/z: 449 [M+H]⁺.

### Example 234: 8-[6-(1,3,4-thiadiazol-2-yl)pyrazin-2-yl]-2-[5-(trifluoromethyl)pyridin-2-yl]-2,8-diazaspiro[4.5]decane

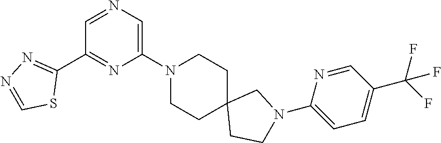 [SMILES: FC(F)(F)c1ccc(N2CCC3(CCN(c4cncc(-c5nncs5)n4)CC3)C2)nc1]

Followed the General Procedure A to afford the desired product as a yellow solid (25 mg, 74%). ¹H NMR (500 MHz, CDCl₃) δ 9.19 (s, 1H), 8.85 (s, 1H), 8.39 (dt, J=2.7, 0.9 Hz, 1H), 8.28 (d, J=0.6 Hz, 1H), 7.62 (dd, J=9.0, 2.5 Hz, 1H), 6.39 (d, J=8.9 Hz, 1H), 3.86 (dt, J=11.4, 5.3 Hz, 2H), 3.67-3.58 (m, 3H), 3.52 (s, 1H), 2.02 (t, J=7.0 Hz, 2H), 1.83-1.72 (m, 4H). MS m/z: 449 [M+H]⁺.

### Example 235: 8-[6-(1,3,4-thiadiazol-2-yl)pyrazin-2-yl]-2-[5-(trifluoromethyl)pyridin-3-yl]-2,8-diazaspiro[4.5]decane

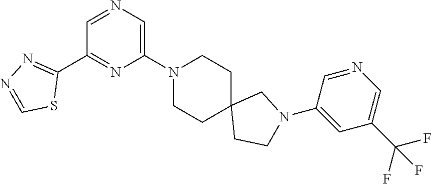 [SMILES: FC(F)(F)c1cncc(N2CCC3(CCN(c4cncc(-c5nncs5)n4)CC3)C2)c1]

Followed the General Procedure A to afford the desired product as a yellow solid (22 mg, 65%). ¹H NMR (500 MHz, CDCl₃) δ 9.19 (s, 1H), 8.86 (s, 1H), 8.29 (d, J=0.6 Hz, 1H), 8.22-8.18 (m, 1H), 8.13 (d, J=2.8 Hz, 1H), 6.95 (t, J=2.4 Hz, 1H), 3.80 (ddd, J=13.6, 6.9, 4.5 Hz, 2H), 3.70 (ddd, J=13.6, 7.3, 4.4 Hz, 2H), 3.50 (t, J=7.0 Hz, 2H), 3.31 (s, 2H), 2.05 (t, J=7.0 Hz, 2H), 1.86-1.74 (m, 4H). MS m/z: 449 [M+H]⁺.

### Example 236: 8-[6-(1,3,4-thiadiazol-2-yl)pyrazin-2-yl]-2-[6-(trifluoromethyl)pyridin-3-yl]-2,8-diazaspiro[4.5]decan-3-one

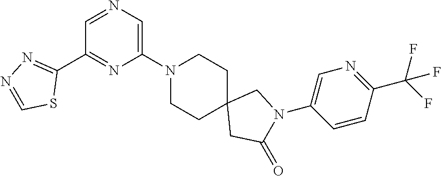 [SMILES: O=C1CC2(CCN(c3cncc(-c4nncs4)n3)CC2)CN1c1ccc(C(F)(F)F)nc1]

Followed the General Procedure A to afford the desired product as a yellow solid (23 mg, 66%). ¹H NMR (500 MHz, CDCl₃) δ 9.20 (s, 1H), 8.89 (d, J=0.6 Hz, 1H), 8.80 (d, J=2.5 Hz, 1H), 8.51-8.45 (m, 1H), 8.31 (d, J=0.6 Hz, 1H), 7.71 (dd, J=8.8, 0.7 Hz, 1H), 3.97 (dt, J=13.8, 5.0 Hz, 2H), 3.80 (s, 2H), 3.57 (ddd, J=13.8, 7.2, 5.3 Hz, 2H), 2.70 (s, 2H), 1.93-1.87 (m, 4H). MS m/z: 463 [M+H]⁺.

### Example 237: 8-[6-(1,3,4-thiadiazol-2-yl)pyrazin-2-yl]-2-[5-(trifluoromethyl)pyridin-3-yl]-2,8-diazaspiro[4.5]decan-3-one

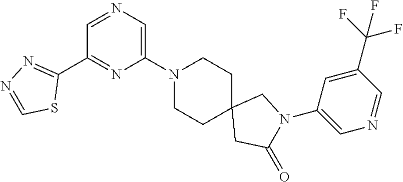 [SMILES: O=C1CC2(CCN(c3cncc(-c4nncs4)n3)CC2)CN1c1cncc(C(F)(F)F)c1]

Followed the General Procedure A to afford the desired product as a yellow oil (19 mg, 55%). ¹H NMR (500 MHz, CDCl₃) δ 9.20 (s, 1H), 8.96 (d, J=2.5 Hz, 1H), 8.89 (d, J=0.6 Hz, 1H), 8.68 (dd, J=2.0, 1.0 Hz, 1H), 8.49 (s, 1H), 8.31 (d, J=0.6 Hz, 1H), 3.97 (dt, J=13.8, 5.1 Hz, 2H), 3.80 (s, 2H), 3.58 (ddd, J=13.2, 7.3, 5.2 Hz, 2H), 2.69 (s, 2H), 1.96-1.79 (m, 4H). MS m/z: 463 [M+H]⁺.

### Example 238: 8-[6-(1H-imidazol-1-yl)pyrazin-2-yl]-2-[6-(trifluoromethyl)pyridin-3-yl]-2,8-diazaspiro[4.5]decane

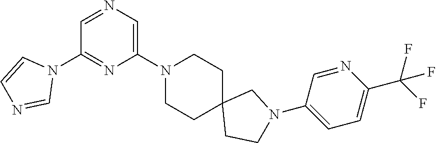 [SMILES: FC(F)(F)c1ccc(N2CCC3(CCN(c4cncc(-n5ccnc5)n4)CC3)C2)cn1]

Followed the General Procedure A to afford the desired product as a colorless oil (26 mg, 97%). ¹H NMR (500 MHz, CDCl₃) δ 8.31 (s, 1H), 8.09 (s, 1H), 8.01 (d, J=2.8 Hz, 1H), 7.99 (s, 1H), 7.58 (d, J=1.6 Hz, 1H), 7.49 (d, J=8.7 Hz, 1H), 7.20 (s, 1H), 6.83 (dd, J=8.7, 2.8 Hz, 1H), 3.74 (m, 4H), 3.51 (t, J=7.0 Hz, 2H), 3.31 (s, 2H), 2.05 (t, J=6.9 Hz, 2H), 1.84-1.72 (m, 4H). MS m/z: 431 [M+H]⁺.

### Example 239: 8-[6-(1H-pyrazol-1-yl)pyrazin-2-yl]-2-[6-(trifluoromethyl)pyridin-3-yl]-2,8-diazaspiro[4.5]decane

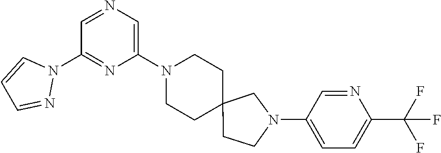 [SMILES: FC(F)(F)c1ccc(N2CCC3(CCN(c4cncc(-n5cccn5)n4)CC3)C2)cn1]

Followed the General Procedure A to afford the desired product as a colorless oil (8 mg, 30%). ¹H NMR (500 MHz, CDCl₃) δ 8.55 (s, 1H), 8.41 (d, J=2.6 Hz, 1H), 8.06 (s, 1H), 8.02 (d, J=2.8 Hz, 1H), 7.76 (d, J=1.6 Hz, 1H), 7.49 (d, J=8.7 Hz, 1H), 6.83 (dd, J=8.7, 2.8 Hz, 1H), 6.46 (t, J=2.2 Hz, 1H), 3.80-3.65 (m, 4H), 3.50 (t, J=7.0 Hz, 2H), 3.31 (s, 2H), 2.04 (t, J=7.0 Hz, 2H), 1.83-1.73 (m, 4H). MS m/z: 431 [M+H]⁺.

### Example 240: 8-{imidazo[1,5-a]pyrazin-8-yl}-2-[6-(trifluoromethyl)pyridin-3-yl]-2,8-diazaspiro[4.5]decane

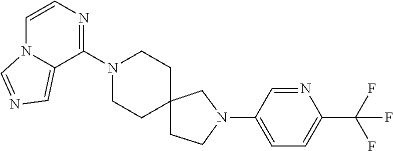 [SMILES: FC(F)(F)c1ccc(N2CCC3(CCN(c4nccn5cncc45)CC3)C2)cn1]

Followed the General Procedure A to afford the desired product as a brown oil (12 mg, 60%). ¹H NMR (500 MHz, CDCl₃) δ 8.07 (s, 1H), 8.02 (d, J=2.8 Hz, 1H), 7.64 (s, 1H), 7.49 (d, J=8.7 Hz, 1H), 7.37 (d, J=4.8 Hz, 1H), 7.15 (d, J=4.8 Hz, 1H), 6.83 (dd, J=8.7, 2.9 Hz, 1H), 3.87 (m, 4H), 3.50 (t, J=7.0 Hz, 2H), 3.31 (s, 2H), 2.05 (dd, J=8.0, 6.0 Hz, 2H), 1.85-1.77 (m, 4H). MS m/z: 403 [M+H]⁺.

### Example 241: 8-[6-(1H-pyrazol-1-yl)pyrazin-2-yl]-2-[4-(trifluoromethyl)pyridin-2-yl]-2,8-diazaspiro[4.5]decan-1-one

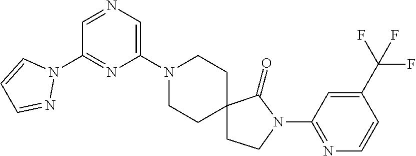 [SMILES: O=C1N(c2cc(C(F)(F)F)ccn2)CCC12CCN(c1cncc(-n3cccn3)n1)CC2]

Followed the General Procedure A to afford the desired product as a white wax (24 mg, 67%). ¹H NMR (500 MHz, CDCl₃) δ 8.76 (s, 1H), 8.57-8.51 (m, 2H), 8.42 (d, J=2.6 Hz, 1H), 8.08 (s, 1H), 7.76 (d, J=1.6 Hz, 1H), 7.27 (s, 1H), 6.46 (s, 1H), 4.26 (dt, J=13.7, 4.8 Hz, 2H), 4.12 (t, J=7.0 Hz, 2H), 3.42 (ddd, J=13.5, 10.2, 3.3 Hz, 2H), 2.18 (t, J=7.1 Hz, 2H), 2.12 (ddd, J=14.0, 10.2, 4.2 Hz, 2H), 1.73 (dd, J=13.7, 4.4 Hz, 2H). MS m/z: 444 [M+H]⁺.

### Example 242: 8-[6-(1H-imidazol-1-yl)pyrazin-2-yl]-2-[4-(trifluoromethyl)pyridin-2-yl]-2,8-diazaspiro[4.5]decan-1-one

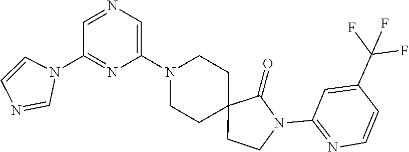 [SMILES: O=C1N(c2cc(C(F)(F)F)ccn2)CCC12CCN(c1cncc(-n3ccnc3)n1)CC2]

Followed the General Procedure A to afford the desired product as a white wax (20 mg, 56%). ¹H NMR (500 MHz, CDCl₃) δ 8.75 (s, 1H), 8.54 (d, J=5.2 Hz, 1H), 8.31 (s, 1H), 8.11 (s, 1H), 8.00 (s, 1H), 7.59 (s, 1H), 7.28 (d, J=1.5 Hz, 1H), 7.20 (s, 1H), 4.26 (dt, J=13.6, 4.8 Hz, 2H), 4.13 (t, J=7.0 Hz, 2H), 3.45 (ddd, J=13.5, 10.0, 3.4 Hz, 2H), 2.19 (t, J=7.0 Hz, 2H), 2.11 (ddd, J=14.0, 10.0, 4.1 Hz, 2H), 1.73 (dt, J=13.6, 4.4 Hz, 2H). MS m/z: 444 [M+H]⁺.

### Example 243: 8-[6-(2,2,2-trifluoroethoxy)pyrazin-2-yl]-2-[6-(trifluoromethyl)pyridin-3-yl]-2,8-diazaspiro[4.5]decane

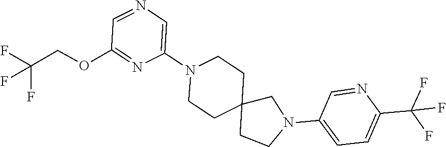 [SMILES: FC(F)(F)COc1cncc(N2CCC3(CCN(c4ccc(C(F)(F)F)nc4)C3)CC2)n1]

Followed the General Procedure A to afford the desired product as a white wax (17 mg, 47%). ¹H NMR (500 MHz, CDCl₃) δ 8.01 (d, J=2.8 Hz, 1H), 7.78 (s, 1H), 7.62 (s, 1H), 7.49 (d, J=8.7 Hz, 1H), 6.82 (dd, J=8.7, 2.8 Hz, 1H), 4.68 (q, J=8.4 Hz, 2H), 3.71-3.55 (m, 4H), 3.49 (t, J=7.0 Hz, 2H), 3.29 (s, 2H), 2.03 (t, J=6.9 Hz, 2H), 1.81-1.70 (m, 4H). MS m/z: 462 [M+H]⁺.

### Example 244: 8-[6-(2,2,2-trifluoroethoxy)pyrazin-2-yl]-2-[4-(trifluoromethyl)pyridin-2-yl]-2,8-diazaspiro[4.5]decan-1-one

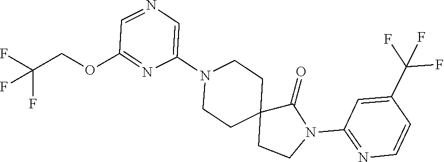 [SMILES: O=C1N(c2cc(C(F)(F)F)ccn2)CCC12CCN(c1cncc(OCC(F)(F)F)n1)CC2]

Followed the General Procedure A to afford the desired product as a off-white wax (14 mg, 40%). ¹H NMR (500 MHz, CDCl₃) δ 8.76 (s, 1H), 8.53 (d, J=5.2 Hz, 1H), 7.80 (s, 1H), 7.63 (s, 1H), 7.27 (s, 1H), 4.69 (q, J=8.5 Hz, 2H), 4.20-4.09 (m, 4H), 3.32 (ddd, J=13.5, 10.2, 3.3 Hz, 2H), 2.17 (t, J=7.1 Hz, 2H), 2.08 (ddd, J=14.1, 10.3, 4.2 Hz, 2H), 1.69 (dt, J=13.7, 4.1 Hz, 2H). MS m/z: 476 [M+H]⁺.

### Example 245: 8-[6-(pyrrolidin-1-yl)pyrazin-2-yl]-2-[4-(trifluoromethyl)pyridin-2-yl]-2,8-diazaspiro[4.5]decan-1-one

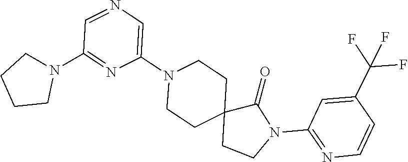 [SMILES: O=C1N(c2cc(C(F)(F)F)ccn2)CCC12CCN(c1cncc(N3CCCC3)n1)CC2]

Followed the General Procedure D to afford the desired product as a colorless oil (5.6 mg, 17%). ¹H NMR (500 MHz, CDCl₃) δ 8.77 (s, 1H), 8.52 (d, J=5.3 Hz, 1H), 7.40 (s, 1H), 7.25 (s, 1H), 7.20 (s, 1H), 4.20 (dt, J=13.6, 4.5 Hz, 2H), 4.09 (t, J=7.1 Hz, 2H), 3.48-3.42 (m, 4H), 3.19 (ddd, J=13.7, 10.8, 3.1 Hz, 2H), 2.16 (t, J=7.1 Hz, 2H), 2.11-2.03 (m, 4H), 2.01-1.97 (m, 4H). MS m/z: 448 [M+H]⁺.

### Example 246: 8-[6-(morpholin-4-yl)pyrazin-2-yl]-2-[4-(trifluoromethyl)pyridin-2-yl]-2,8-diazaspiro[4.5]decan-1-one

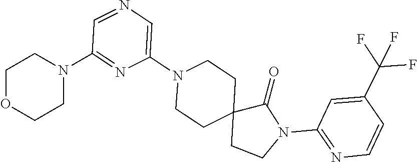 [SMILES: O=C1N(c2cc(C(F)(F)F)ccn2)CCC12CCN(c1cncc(N3CCOCC3)n1)CC2]

Followed the General Procedure D to afford the desired product as a colorless oil (8 mg, 23%). ¹H NMR (500 MHz, CDCl₃) δ 8.76 (s, 1H), 8.52 (d, J=5.2 Hz, 1H), 7.54 (s, 1H), 7.43 (s, 1H), 7.25 (s, 1H), 4.17 (dt, J=13.6, 4.5 Hz, 2H), 4.09 (t, J=7.1 Hz, 2H), 3.82 (q, J=4.6 Hz, 5H), 3.51 (q, J=5.1 Hz, 5H), 3.22 (ddd, J=13.6, 10.7, 3.2 Hz, 2H), 2.16 (t, J=7.1 Hz, 2H), 2.06 (ddd, J=14.4, 10.7, 4.2 Hz, 2H). MS m/z: 464 [M+H]⁺.

### Example 247: 8-[1-(2,2-difluoroethyl)-1H-pyrazolo[3,4-b]pyrazin-6-yl]-4-phenyl-2-[6-(trifluoromethyl)pyridin-3-yl]-2,8-diazaspiro[4.5]decan-1-one

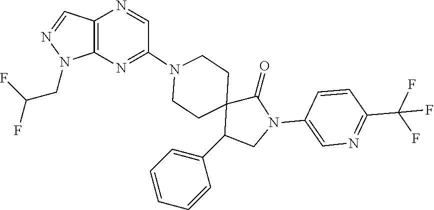 [SMILES: O=C1N(c2ccc(C(F)(F)F)nc2)CC(c2ccccc2)C12CCN(c1cnc3cnn(CC(F)F)c3n1)CC2]

Followed the General Procedure A to afford the desired product as a colorless oil (11 mg, 58%). ¹H NMR (500 MHz, CDCl₃) δ 8.93 (d, J=2.5 Hz, 1H), 8.58 (dd, J=8.7, 2.5 Hz, 1H), 8.15 (s, 1H), 8.01 (s, 1H), 7.75 (d, J=8.7 Hz, 1H), 7.39-7.30 (m, 3H), 7.24-7.19 (m, 2H), 6.18 (tt, J=55.6, 4.5 Hz, 1H), 4.62 (td, J=13.3, 4.5 Hz, 2H), 4.30 (dd, J=10.0, 7.3 Hz, 1H), 4.20-4.11 (m, 2H), 4.03 (dt, J=13.5, 5.0 Hz, 1H), 3.86-3.73 (m, 2H), 3.53 (dd, J=7.3, 5.5 Hz, 1H), 2.17-2.10 (m, 1H), 1.95-1.83 (m, 2H), 1.43 (ddd, J=13.7, 9.0, 4.3 Hz, 1H). MS m/z: 559 [M+H]⁺.

### Example 248: 8-[6-(pyrrolidin-1-yl)pyrazin-2-yl]-2-[6-(trifluoromethyl)pyridin-3-yl]-2,8-diazaspiro[4.5]decane

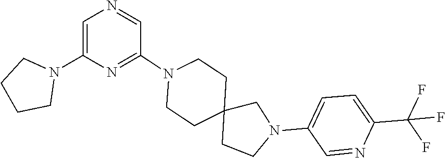 [SMILES: FC(F)(F)c1ccc(N2CCC3(CCN(c4cncc(N5CCCC5)n4)CC3)C2)cn1]

Followed the General Procedure D to afford the desired product as a light-yellow oil (3.2 mg, 16%). MS m/z: 433 [M+H]⁺.

### Example 249: 8-[6-(morpholin-4-yl)pyrazin-2-yl]-2-[6-(trifluoromethyl)pyridin-3-yl]-2,8-diazaspiro[4.5]decane

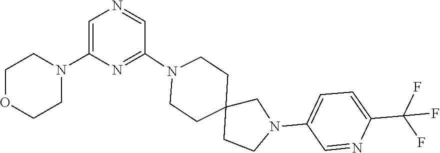 [SMILES: FC(F)(F)c1ccc(N2CCC3(CCN(c4cncc(N5CCOCC5)n4)CC3)C2)cn1]

Followed the General Procedure D to afford the desired product as a light-brown solid (7.7 mg, 23%). ¹H NMR (500 MHz, CDCl₃) δ 8.00 (d, J=2.9 Hz, 1H), 7.52 (d, J=2.1 Hz, 1H), 7.48 (dd, J=8.7, 2.1 Hz, 1H), 7.42 (d, J=2.1 Hz, 1H), 6.86-6.77 (m, 1H), 3.87-3.76 (m, 4H), 3.68-3.52 (m, 4H), 3.48 (dt, J=13.5, 5.6 Hz, 5H), 3.27 (d, J=2.0 Hz, 2H), 2.07-1.96 (m, 3H), 1.72 (q, J=5.7 Hz, 4H). MS m/z: 450 [M+H]⁺.

### Example 250: 8-(6-(2-methyl-1H-imidazol-1-yl)pyrazin-2-yl)-2-(6-(trifluoromethyl)pyridine-3-yl)-2,8-diazaspiro[4.5]decane

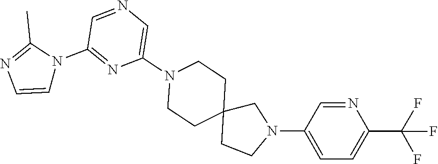 [SMILES: Cc1nccn1-c1cncc(N2CCC3(CCN(c4ccc(C(F)(F)F)nc4)C3)CC2)n1]

Followed the General Procedure A to afford the desired product as a light-brown solid (20 mg, 73%). ¹H NMR (500 MHz, CDCl₃) δ 8.12 (d, J=0.6 Hz, 1H), 8.02 (d, J=2.8 Hz, 1H), 7.93 (d, J=0.6 Hz, 1H), 7.49 (d, J=8.7 Hz, 1H), 7.28 (d, J=1.6 Hz, 1H), 7.03 (d, J=1.5 Hz, 1H), 6.83 (dd, J=8.6, 2.8 Hz, 1H), 3.76 (ddd, J=13.6, 6.9, 4.5 Hz, 2H), 3.68 (ddd, J=13.6, 7.2, 4.4 Hz, 2H), 3.50 (t, J=7.0 Hz, 2H), 3.31 (s, 2H), 2.63 (s, 3H), 2.04 (t, J=7.0 Hz, 2H), 1.84-1.71 (m, 4H). MS m/z: 445 [M+H]⁺.

### Example 251: 8-(6-phenylpyrazin-2-yl)-2-[6-(trifluoromethyl)pyridin-3-yl]-2,8-diazaspiro[4.5]decane

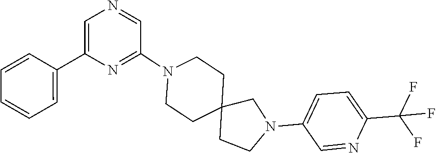 [SMILES: FC(F)(F)c1ccc(N2CCC3(CCN(c4cncc(-c5ccccc5)n4)CC3)C2)cn1]

Followed the General Procedure A to afford the desired product as a light-yellow solid (12 mg, 44%). ¹H NMR (500 MHz, CDCl₃) δ 8.35 (s, 1H), 8.13 (s, 1H), 8.05-7.96 (m, 3H), 7.51-7.40 (m, 4H), 6.83 (dd, J=8.6, 2.8 Hz, 1H), 3.76 (m, 5H), 3.50 (t, J=7.0 Hz, 2H), 3.31 (s, 2H), 2.04 (t, J=7.0 Hz, 2H), 1.86-1.73 (m, 4H). MS m/z: 441 [M+H]⁺.

### Example 252: 8-[6-(pyrimidin-2-yl)pyrazin-2-yl]-2-[6-(trifluoromethyl)pyridin-3-yl]-2,8-diazaspiro[4.5]decane

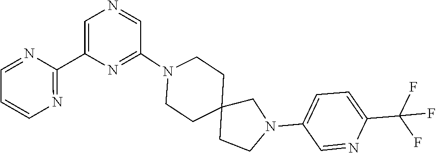 [SMILES: FC(F)(F)c1ccc(N2CCC3(CCN(c4cncc(-c5ncccn5)n4)CC3)C2)cn1]

Followed the General Procedure A to afford the desired product as a light-yellow solid (10 mg, 36%). ¹H NMR (500 MHz, CDCl₃) δ 8.95 (s, 1H), 8.92 (d, J=4.9 Hz, 2H), 8.32 (s, 1H), 8.01 (d, J=2.8 Hz, 1H), 7.48 (d, J=8.7 Hz, 1H), 7.33 (t, J=4.8 Hz, 1H), 6.82 (dd, J=8.7, 2.9 Hz, 1H), 3.79 (qdd, J=13.2, 9.1, 4.6 Hz, 4H), 3.50 (t, J=7.0 Hz, 2H), 3.30 (s, 2H), 2.04 (t, J=7.0 Hz, 2H), 1.86-1.75 (m, 4H). MS m/z: 443 [M+H]⁺.

### Example 253: 8-[6-(1H-1,2,4-triazol-1-yl)pyrazin-2-yl]-2-[6-(trifluoromethyl)pyridin-3-yl]-2,8-diazaspiro[4.5]decane

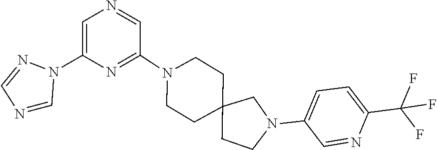 [SMILES: FC(F)(F)c1ccc(N2CCC3(CCN(c4cncc(-n5cncn5)n4)CC3)C2)cn1]

Followed the General Procedure A to afford the desired product as a white solid (12 mg, 45%). ¹H NMR (500 MHz, CDCl₃) δ 9.02 (s, 1H), 8.45 (s, 1H), 8.19 (s, 1H), 8.12 (s, 1H), 8.02 (d, J=2.8 Hz, 1H), 7.49 (d, J=8.7 Hz, 1H), 6.83 (dd, J=8.7, 2.9 Hz, 1H), 3.75 (m, 4H), 3.51 (t, J=7.0 Hz, 2H), 3.32 (s, 2H), 2.05 (t, J=7.0 Hz, 2H), 1.85-1.75 (m, 4H). MS m/z: 431 [M+H]⁺.

### Example 254: 8-[6-(propan-2-yloxy)pyrazin-2-yl]-2-[6-(trifluoromethyl)pyridin-3-yl]-2,8-diazaspiro[4.5]decane

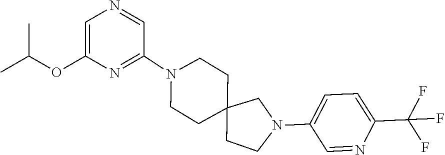 [SMILES: CC(C)Oc1cncc(N2CCC3(CCN(c4ccc(C(F)(F)F)nc4)C3)CC2)n1]

Followed the General Procedure A to afford the desired product as a white solid (9.7 mg, 32%). ¹H NMR (500 MHz, CDCl₃) δ 8.01 (d, J=2.8 Hz, 1H), 7.64 (s, 1H), 7.49 (s, 1H), 7.47 (d, J=1.5 Hz, 1H), 6.82 (dd, J=8.7, 2.9 Hz, 1H), 5.17 (p, J=6.2 Hz, 1H), 3.60 (m, 4H), 3.48 (t, J=7.0 Hz, 2H), 3.28 (s, 2H), 2.01 (t, J=7.0 Hz, 2H), 1.79-1.68 (m, 4H), 1.34 (d, J=6.2 Hz, 6H). MS m/z: 423 [M+H]⁺.

### Example 255: 8-[6-(trifluoromethyl)pyrazin-2-yl]-2-[6-(trifluoromethyl)pyridin-3-yl]-2,8-diazaspiro[4.5]decane

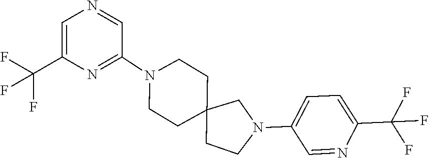 [SMILES: FC(F)(F)c1ccc(N2CCC3(CCN(c4cncc(C(F)(F)F)n4)CC3)C2)cn1]

Followed the General Procedure A to afford the desired product as a light-yellow oil (20 mg, 75%). ¹H NMR (500 MHz, CDCl₃) δ 8.33 (s, 1H), 8.15 (s, 1H), 8.01 (d, J=2.8 Hz, 1H), 7.49 (d, J=8.7 Hz, 1H), 6.82 (dd, J=8.7, 2.9 Hz, 1H), 3.81-3.66 (m, 4H), 3.50 (t, J=7.0 Hz, 2H), 3.30 (s, 2H), 2.04 (t, J=7.0 Hz, 2H), 1.83-1.70 (m, 4H). MS m/z: 432 [M+H]⁺.

### Example 256: 8-[6-(piperidin-1-yl)pyrazin-2-yl]-2-[6-(trifluoromethyl)pyridin-3-yl]-2,8-diazaspiro[4.5]decane

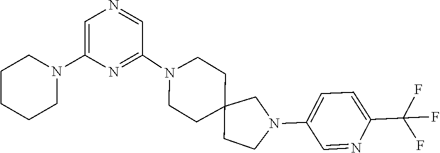 [SMILES: FC(F)(F)c1ccc(N2CCC3(CCN(c4cncc(N5CCCCC5)n4)CC3)C2)cn1]

Followed the General Procedure A to afford the desired product as a light-yellow oil (4 mg, 50%). ¹H NMR (500 MHz, CDCl₃) δ 8.00 (d, J=2.8 Hz, 1H), 7.52-7.44 (m, 2H), 7.42 (s, 1H), 6.81 (dd, J=8.7, 2.9 Hz, 1H), 3.65-3.58 (m, 2H), 3.57-3.43 (m, 8H), 3.26 (s, 2H), 2.00 (t, J=7.0 Hz, 2H), 1.76-1.68 (m, 4H), 1.68-1.58 (m, 6H). MS m/z: 447 [M+H]⁺.

### Example 257: 8-[1-(2,2-difluoroethyl)-3-methyl-1H-pyrazolo[3,4-b]pyrazin-6-yl]-2-[6-(trifluoromethyl)pyridin-3-yl]-2,8-diazaspiro[4.5]decane

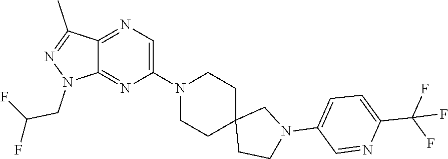 [SMILES: Cc1nn(CC(F)F)c2nc(N3CCC4(CCN(c5ccc(C(F)(F)F)nc5)C4)CC3)cnc12]

Followed the General Procedure A to afford the desired product as a light-yellow solid (22 mg, 74%). ¹H NMR (500 MHz, CDCl₃) δ 8.18 (s, 1H), 8.02 (d, J=2.8 Hz, 1H), 7.49 (d, J=8.7 Hz, 1H), 6.83 (dd, J=8.7, 2.8 Hz, 1H), 6.19 (tt, J=55.7, 4.5 Hz, 1H), 4.59 (td, J=13.4, 4.5 Hz, 2H), 3.80 (m, 4H), 3.50 (t, J=7.0 Hz, 2H), 3.31 (s, 2H), 2.55 (s, 3H), 2.04 (t, J=7.0 Hz, 2H), 1.84-1.73 (m, 4H), 1.59 (s, 2H). MS m/z: 483 [M+H]⁺.

### Example 258: 8-[1-(2,2-difluoroethyl)-3-methyl-1H-pyrazolo[3,4-b]pyrazin-6-yl]-2-[6-(trifluoromethyl)pyridin-3-yl]-2,8-diazaspiro[4.5]decan-3-one

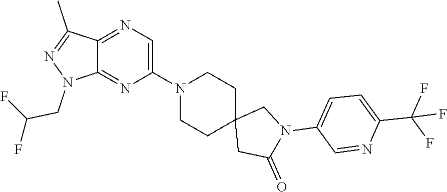 [SMILES: Cc1nn(CC(F)F)c2nc(N3CCC4(CC3)CC(=O)N(c3ccc(C(F)(F)F)nc3)C4)cnc12]

Followed the General Procedure A to afford the desired product as a white solid (21 mg, 68%). ¹H NMR (500 MHz, CDCl₃) δ 8.80 (d, J=2.6 Hz, 1H), 8.47 (dd, J=8.7, 2.6 Hz, 1H), 8.19 (s, 1H), 7.71 (d, J=8.7 Hz, 1H), 6.19 (tt, J=55.7, 4.5 Hz, 1H), 4.59 (td, J=13.4, 4.5 Hz, 2H), 4.03 (dt, J=13.9, 5.0 Hz, 2H), 3.79 (s, 2H), 3.61 (dt, J=13.2, 6.2 Hz, 2H), 2.69 (s, 2H), 2.55 (s, 3H), 1.89 (t, J=5.6 Hz, 4H). MS m/z: 497 [M+H]⁺.

### Example 259: 1′-[1-(2,2-difluoroethyl)-1H-pyrazolo[3,4-b]pyrazin-6-yl]-1-[4-(trifluoromethyl)pyridin-2-yl]-1,2-dihydrospiro[indole-3,4′-piperidine]

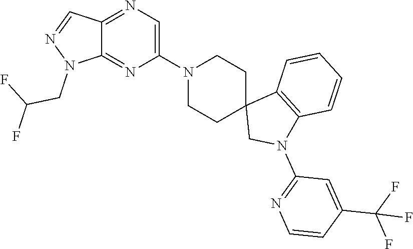 [SMILES: FC(F)Cn1ncc2ncc(N3CCC4(CC3)CN(c3cc(C(F)(F)F)ccn3)c3ccccc34)nc21]

Followed the General Procedure A to afford the desired product as a light-yellow oil (22 mg, 79%). ¹H NMR (500 MHz, CDCl₃) δ 8.51 (d, J=5.1 Hz, 1H), 8.32 (s, 1H), 8.17 (d, J=8.1 Hz, 1H), 8.07 (s, 1H), 7.30-7.27 (m, 1H), 7.17 (dd, J=7.5, 1.3 Hz, 1H), 7.06 (s, 1H), 7.03-6.96 (m, 2H), 6.23 (tt, J=55.6, 4.5 Hz, 1H), 4.68 (td, J=13.3, 4.5 Hz, 2H), 4.62-4.55 (m, 2H), 4.09 (s, 2H), 3.29 (ddd, J=14.2, 12.7, 2.7 Hz, 2H), 2.09 (td, J=13.3, 4.5 Hz, 2H), 1.94 (d, J=13.8 Hz, 2H). MS m/z: 517 [M+H]⁺.

### Example 260: 8-[1-(2,2-difluoroethyl)-3-methyl-1H-pyrazolo[3,4-b]pyrazin-6-yl]-2-[6-(trifluoromethyl)pyridin-3-yl]-2,8-diazaspiro[4.5]decan-1-one

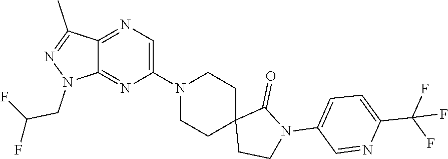 [SMILES: Cc1nn(CC(F)F)c2nc(N3CCC4(CC3)CCN(c3ccc(C(F)(F)F)nc3)C4=O)cnc12]

Followed the General Procedure A to afford the desired product as a white solid (24 mg, 78%). ¹H NMR (500 MHz, CDCl₃) δ 8.40 (d, J=2.9 Hz, 1H), 8.22 (s, 1H), 8.03 (s, 1H), 7.41 (td, J=8.3, 2.9 Hz, 1H), 7.32 (dd, J=8.6, 4.4 Hz, 1H), 6.20 (tt, J=55.6, 4.5 Hz, 1H), 4.65 (td, J=13.3, 4.5 Hz, 2H), 4.59 (s, 2H), 3.83 (ddd, J=13.8, 6.7, 4.1 Hz, 2H), 3.65 (ddd, J=13.7, 7.9, 4.0 Hz, 2H), 3.31 (s, 2H), 2.45 (s, 2H), 1.80-1.70 (m, 4H). MS m/z: 497 [M+H]⁺.

### Example 261: 1′-[1-(2,2-difluoroethyl)-1H-pyrazolo[3,4-b]pyrazin-6-yl]-1-[6-(trifluoromethyl)pyridin-3-yl]-1,2-dihydrospiro[indole-3,4′-piperidin]-2-one

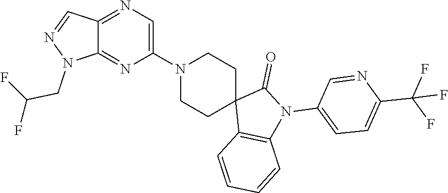 [SMILES: O=C1N(c2ccc(C(F)(F)F)nc2)c2ccccc2C12CCN(c1cnc3cnn(CC(F)F)c3n1)CC2]

Followed the General Procedure A to afford the desired product as a white solid (24 mg, 84%). ¹H NMR (500 MHz, CDCl₃) δ 8.92 (d, J=2.4 Hz, 1H), 8.33 (s, 1H), 8.07 (d, J=8.3 Hz, 2H), 7.90 (dd, J=8.4, 0.8 Hz, 1H), 7.36 (dd, J=7.5, 1.3 Hz, 1H), 7.31 (td, J=7.8, 1.2 Hz, 1H), 7.20 (td, J=7.5, 1.0 Hz, 1H), 6.99 (d, J=7.9 Hz, 1H), 6.24 (tt, J=55.6, 4.5 Hz, 1H), 4.69 (td, J=13.3, 4.5 Hz, 2H), 4.34 (dt, J=13.8, 4.6 Hz, 2H), 4.14 (ddd, J=13.8, 8.4, 5.3 Hz, 2H), 2.18-2.08 (m, 4H). MS m/z: 531 [M+H]⁺.

### Example 262: 8-[6-(2-ethyl-1H-imidazol-1-yl)pyrazin-2-yl]-2-[4-(trifluoromethyl)pyridin-2-yl]-2,8-diazaspiro[4.5]decan-1-one

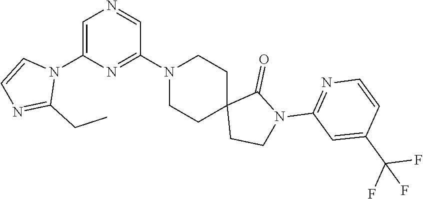 [SMILES: CCc1nccn1-c1cncc(N2CCC3(CC2)CCN(c2cc(C(F)(F)F)ccn2)C3=O)n1]

Followed the General Procedure A to afford the desired product as a colorless oil (22 mg, 63%). ¹H NMR (500 MHz, CDCl₃) δ 8.77-8.73 (m, 1H), 8.53 (d, J=5.2 Hz, 1H), 8.14 (s, 1H), 7.91 (s, 1H), 7.27 (d, J=1.5 Hz, 1H), 7.24 (d, J=1.5 Hz, 1H), 7.06 (d, J=1.5 Hz, 1H), 4.24 (dt, J=13.7, 4.8 Hz, 2H), 4.12 (dd, J=8.0, 6.1 Hz, 2H), 3.44 (ddd, J=13.5, 10.0, 3.4 Hz, 2H), 2.98 (q, J=7.5 Hz, 2H), 2.18 (t, J=7.0 Hz, 2H), 2.10 (ddd, J=13.9, 10.0, 4.1 Hz, 2H), 1.72 (dt, J=13.7, 4.3 Hz, 2H), 1.33 (t, J=7.5 Hz, 3H). MS m/z: 473 [M+H]⁺.

### Example 263: 8-[6-(2-ethyl-1H-imidazol-1-yl)pyrazin-2-yl]-2-[6-(trifluoromethyl)pyridin-3-yl]-2,8-diazaspiro[4.5]decane

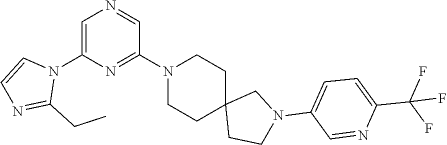 [SMILES: CCc1nccn1-c1cncc(N2CCC3(CCN(c4ccc(C(F)(F)F)nc4)C3)CC2)n1]

Followed the General Procedure A to afford the desired product as a colorless oil (26 mg, 73%). ¹H NMR (500 MHz, CDCl₃) δ 8.13 (s, 1H), 8.02 (d, J=2.8 Hz, 1H), 7.91 (s, 1H), 7.49 (d, J=8.7 Hz, 1H), 7.24 (d, J=1.5 Hz, 1H), 7.06 (d, J=1.5 Hz, 1H), 6.83 (dd, J=8.7, 2.9 Hz, 1H), 3.72 (m, 4H), 3.50 (t, J=7.0 Hz, 2H), 3.31 (s, 2H), 2.98 (q, J=7.5 Hz, 2H), 2.04 (t, J=7.0 Hz, 2H), 1.83-1.72 (m, 4H), 1.34 (t, J=7.5 Hz, 3H). MS m/z: 459 [M+H]⁺.

### Example 264: 8-{imidazo[1,5-a]pyrazin-8-yl}-2-[4-(trifluoromethyl)pyridin-2-yl]-2,8-diazaspiro[4.5]decan-1-one

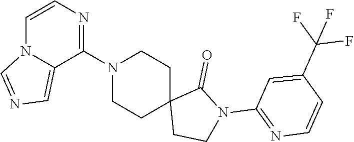 [SMILES: O=C1N(c2cc(C(F)(F)F)ccn2)CCC12CCN(c1nccn3cncc13)CC2]

Followed the General Procedure A to afford the desired product as a colorless oil (19 mg, 61%). ¹H NMR (500 MHz, CDCl₃) δ 8.78-8.75 (m, 1H), 8.53 (d, J=5.2 Hz, 1H), 8.09 (s, 1H), 7.66 (s, 1H), 7.36 (dd, J=4.8, 1.0 Hz, 1H), 7.25 (d, J=2.0 Hz, 1H), 7.16 (d, J=4.8 Hz, 1H), 4.41 (dt, J=13.6, 4.6 Hz, 2H), 4.11 (t, J=7.0 Hz, 2H), 3.55 (ddd, J=13.6, 10.3, 3.1 Hz, 2H), 2.22-2.14 (m, 4H), 1.73 (dt, J=14.8, 4.2 Hz, 2H). MS m/z: 417 [M+H]⁺.

### Example 265: 8-[1-(2,2-difluoroethyl)-1H-pyrazolo[3,4-b]pyrazin-6-yl]-3-methyl-2-[6-(trifluoromethyl)pyridin-3-yl]-2,8-diazaspiro[4.5]decane

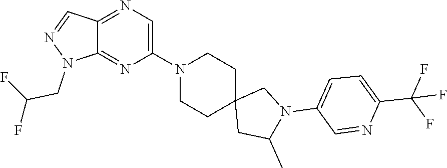 [SMILES: CC1CC2(CCN(c3cnc4cnn(CC(F)F)c4n3)CC2)CN1c1ccc(C(F)(F)F)nc1]

Followed the General Procedure A to afford the desired product as a colorless oil (19 mg, 66%). ¹H NMR (500 MHz, CDCl₃) δ 8.24 (s, 1H), 8.07 (d, J=2.9 Hz, 1H), 8.04 (s, 1H), 7.49 (d, J=8.7 Hz, 1H), 6.89 (dd, J=8.7, 2.9 Hz, 1H), 6.21 (tt, J=55.6, 4.5 Hz, 1H), 4.65 (td, J=13.3, 4.5 Hz, 2H), 4.06 (q, J=6.6 Hz, 1H), 3.91-3.65 (m, 4H), 3.45-3.35 (m, 2H), 2.33 (dd, J=12.9, 7.5 Hz, 1H), 1.92-1.79 (m, 2H), 1.69 (dd, J=12.9, 7.0 Hz, 1H), 1.66-1.61 (m, 2H), 1.32 (d, J=6.0 Hz, 3H). MS m/z: 482 [M+H]⁺.

### Example 266: 8-[1-(2,2-difluoroethyl)-1H-pyrazolo[3,4-b]pyrazin-6-yl]-6,6-difluoro-2-[6-(trifluoromethyl)pyridin-3-yl]-2,8-diazaspiro[4.5]decane

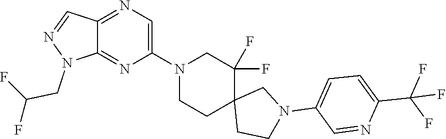 [SMILES: FC(F)Cn1ncc2ncc(N3CCC4(CCN(c5ccc(C(F)(F)F)nc5)C4)C(F)(F)C3)nc21]

Followed the General Procedure A to afford the desired product as a colorless oil (4 mg, 14%). ¹H NMR (500 MHz, CDCl₃) δ 8.30 (s, 1H), 8.10 (s, 1H), 8.05 (d, J=2.9 Hz, 1H), 7.52 (d, J=8.7 Hz, 1H), 6.87 (dd, J=8.7, 2.9 Hz, 1H), 6.21 (tt, J=55.5, 4.5 Hz, 1H), 4.69 (td, J=13.3, 4.4 Hz, 2H), 4.17 (td, J=15.1, 6.6 Hz, 1H), 4.05-3.92 (m, 2H), 3.87-3.73 (m, 2H), 3.63-3.53 (m, 2H), 3.33 (d, J=10.1 Hz, 1H), 2.52 (dt, J=13.1, 8.0 Hz, 1H), 2.10 (ddd, J=12.7, 7.2, 5.0 Hz, 1H), 2.06-1.98 (m, 2H). MS m/z: 504 [M+H]⁺.

### Example 267: 8-[5-methyl-3-(3-methylphenyl)-1,2-oxazole-4-carbonyl]-2-[4-(trifluoromethyl)pyridin-2-yl]-2,8-diazaspiro[4.5]decan-1-one

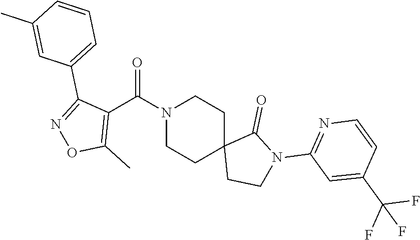 [SMILES: Cc1cccc(-c2noc(C)c2C(=O)N2CCC3(CC2)CCN(c2cc(C(F)(F)F)ccn2)C3=O)c1]

Followed the General Procedure G to afford the desired product as a colorless oil (21 mg, 70%). ¹H NMR (500 MHz, CDCl₃) δ 8.69 (s, 1H), 8.50 (dt, J=5.2, 0.8 Hz, 1H), 7.55 (td, J=1.8, 0.9 Hz, 1H), 7.52 (d, J=7.7 Hz, 1H), 7.38 (t, J=7.6 Hz, 1H), 7.31 (d, J=7.7 Hz, 1H), 7.25 (d, J=1.6 Hz, 1H), 4.41 (s, 1H), 4.09-3.97 (m, 2H), 3.66 (d, J=13.2 Hz, 1H), 3.52 (s, 1H), 3.14 (s, 1H), 2.43 (s, 3H), 2.36 (s, 3H), 2.05 (dtd, J=14.2, 10.0, 9.4, 5.2 Hz, 4H), 1.75-1.63 (m, 2H). MS m/z: 500 [M+H]⁺.

### Example 268: 8-[1-(3-fluoro-5-methylphenyl)-1H-1,2,3-triazole-4-carbonyl]-2-[4-(trifluoromethyl)pyridin-2-yl]-2,8-diazaspiro[4.5]decan-1-one

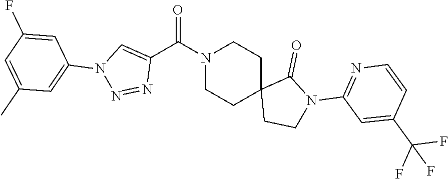 [SMILES: Cc1cc(F)cc(-n2cc(C(=O)N3CCC4(CC3)CCN(c3cc(C(F)(F)F)ccn3)C4=O)nn2)c1]

Followed the General Procedure G to afford the desired product as a colorless oil (20 mg, 67%). ¹H NMR (500 MHz, CDCl₃) δ 8.75 (dt, J=1.7, 0.9 Hz, 1H), 8.53 (d, J=5.2 Hz, 1H), 8.51 (s, 1H), 7.38-7.32 (m, 2H), 7.27 (d, J=1.7 Hz, 1H), 7.01 (dd, J=9.6, 1.4 Hz, 1H), 4.95 (dt, J=14.1, 5.0 Hz, 1H), 4.47-4.39 (m, 1H), 4.11 (t, J=7.0 Hz, 2H), 3.95 (ddd, J=13.5, 9.3, 3.1 Hz, 1H), 3.50 (ddd, J=13.3, 9.5, 3.5 Hz, 1H), 2.51-2.43 (m, 3H), 2.19 (td, J=6.9, 1.8 Hz, 2H), 2.11 (tdd, J=13.9, 9.5, 4.0 Hz, 2H), 1.75 (td, J=14.2, 12.1, 7.5 Hz, 2H). MS m/z: 504 [M+H]⁺.

### Example 269: 8-(3-cyclopropylquinoline-2-carbonyl)-2-[4-(trifluoromethyl)pyridin-2-yl]-2,8-diazaspiro[4.5]decan-1-one

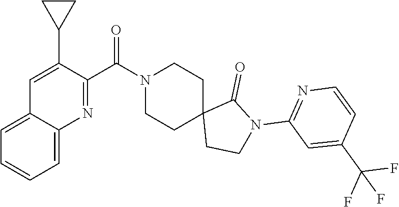 [SMILES: O=C(c1nc2ccccc2cc1C1CC1)N1CCC2(CC1)CCN(c1cc(C(F)(F)F)ccn1)C2=O]

Followed the General Procedure G to afford the desired product as a colorless oil (20 mg, 68%). ¹H NMR (500 MHz, CDCl₃) δ 8.74 (s, 1H), 8.52 (d, J=5.1 Hz, 1H), 8.06 (d, J=8.5 Hz, 1H), 7.75 (dd, J=8.2, 1.4 Hz, 1H), 7.72 (s, 1H), 7.66 (ddd, J=8.5, 6.8, 1.4 Hz, 1H), 7.54 (ddd, J=8.1, 6.8, 1.2 Hz, 1H), 7.26 (s, 1H), 4.56 (dt, J=13.6, 4.9 Hz, 1H), 4.09 (dd, J=7.8, 6.3 Hz, 2H), 3.66-3.52 (m, 2H), 3.27 (ddd, J=13.6, 9.9, 3.5 Hz, 1H), 2.24-2.15 (m, 2H), 2.15-2.08 (m, 2H), 2.06-1.96 (m, 1H), 1.84-1.76 (m, 1H), 1.14 (dddd, J=18.2, 8.6, 6.0, 4.5 Hz, 3H), 0.91 (dd, J=9.7, 4.8 Hz, 1H), 0.80-0.71 (m, 1H). MS m/z: 496 [M+H]⁺.

### Example 270: 8-(4-methyl-1-phenyl-1H-pyrazole-3-carbonyl)-2-[4-(trifluoromethyl)pyridin-2-yl]-2,8-diazaspiro[4.5]decan-1-one

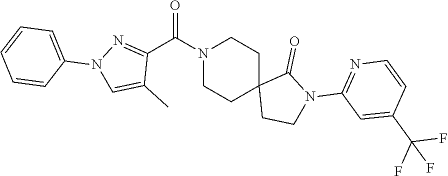 [SMILES: Cc1cn(-c2ccccc2)nc1C(=O)N1CCC2(CC1)CCN(c1cc(C(F)(F)F)ccn1)C2=O]

Followed the General Procedure G to afford the desired product as a colorless oil (20 mg, 69%). ¹H NMR (500 MHz, CDCl₃) δ 8.79-8.74 (m, 1H), 8.52 (dd, J=5.1, 0.7 Hz, 1H), 7.75 (t, J=0.9 Hz, 1H), 7.68-7.62 (m, 2H), 7.47-7.41 (m, 2H), 7.31-7.27 (m, 1H), 7.25 (d, J=1.5 Hz, 1H), 4.49 (dt, J=13.3, 4.8 Hz, 1H), 4.40 (dt, J=13.8, 4.8 Hz, 1H), 4.10 (t, J=7.0 Hz, 2H), 3.55 (ddd, J=13.6, 10.0, 3.2 Hz, 1H), 3.41 (ddd, J=13.4, 10.0, 3.4 Hz, 1H), 2.27 (d, J=0.9 Hz, 3H), 2.24-2.05 (m, 4H), 1.73 (d, J=13.8 Hz, 1H), 1.66 (d, J=13.5 Hz, 1H). MS m/z: 485 [M+H]⁺.

### Example 271: 8-[1-(2,2-difluoroethyl)-3-methyl-1H-pyrazolo[3,4-b]pyrazin-6-yl]-2-[6-(trifluoromethyl)pyrazin-2-yl]-2,8-diazaspiro[4.5]decan-1-one

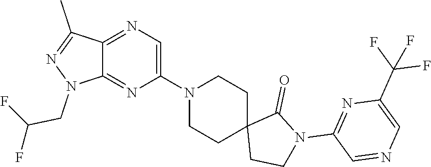 [SMILES: Cc1nn(CC(F)F)c2nc(N3CCC4(CC3)CCN(c3cncc(C(F)(F)F)n3)C4=O)cnc12]

Followed the General Procedure D to afford the desired product as a colorless oil (14 mg, 71%). ¹H NMR (500 MHz, CDCl₃) δ 9.97 (s, 1H), 8.68 (s, 1H), 8.19 (s, 1H), 6.19 (tt, J=55.7, 4.5 Hz, 1H), 4.59 (td, J=13.4, 4.5 Hz, 2H), 4.36 (dt, J=13.8, 4.7 Hz, 2H), 4.13-4.07 (m, 2H), 3.48 (ddd, J=13.6, 10.2, 3.3 Hz, 2H), 2.55 (s, 3H), 2.27-2.22 (m, 2H), 2.13 (ddd, J=14.0, 10.2, 4.1 Hz, 2H), 1.75 (dtd, J=13.4, 3.6, 1.7 Hz, 2H). MS m/z: 497 [M+H]⁺.

### Example 272: 8-[1-(2,2,2-trifluoroethyl)-1H-pyrazolo[3,4-b]pyrazin-6-yl]-2-[6-(trifluoromethyl)pyrazin-2-yl]-2,8-diazaspiro[4.5]decan-1-one

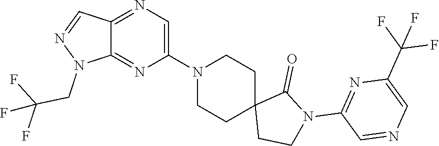 [SMILES: O=C1N(c2cncc(C(F)(F)F)n2)CCC12CCN(c1cnc3cnn(CC(F)(F)F)c3n1)CC2]

Followed the General Procedure D to afford the desired product as a colorless oil (16 mg, 57%). ¹H NMR (500 MHz, CDCl₃) δ 9.97 (s, 1H), 8.68 (s, 1H), 8.28 (d, J=1.9 Hz, 1H), 8.09 (d, J=1.9 Hz, 1H), 4.89 (q, J=8.4 Hz, 2H), 4.36 (dd, J=12.1, 6.7 Hz, 2H), 4.15-4.06 (m, 2H), 3.52 (ddt, J=13.0, 10.2, 2.6 Hz, 2H), 2.29-2.19 (m, 2H), 2.13 (td, J=11.9, 10.1, 3.9 Hz, 2H), 1.75 (dt, J=13.4, 4.1 Hz, 2H). MS m/z: 501 [M+H]⁺.

### Example 273: 8-[1-(oxetan-3-yl)-1H-pyrazolo[3,4-b]pyrazin-6-yl]-2-[4-(trifluoromethyl)pyridin-2-yl]-2,8-diazaspiro[4.5]decan-1-one

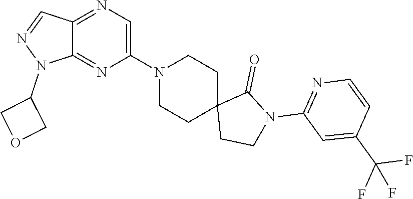 [SMILES: O=C1N(c2cc(C(F)(F)F)ccn2)CCC12CCN(c1cnc3cnn(C4COC4)c3n1)CC2]

Followed the General Procedure A to afford the desired product as a colorless oil (14 mg, 50%). ¹H NMR (500 MHz, CDCl₃) δ 8.75 (dt, J=1.6, 0.8 Hz, 1H), 8.54 (dt, J=5.1, 0.8 Hz, 1H), 8.26 (s, 1H), 8.10 (s, 1H), 7.27 (dd, J=5.0, 1.4 Hz, 1H), 5.95 (tt, J=8.0, 6.7 Hz, 1H), 5.34 (t, J=6.7 Hz, 2H), 5.09-5.03 (m, 2H), 4.36 (dt, J=13.7, 4.7 Hz, 2H), 4.13 (dd, J=7.5, 6.6 Hz, 2H), 3.50 (ddd, J=13.5, 10.1, 3.3 Hz, 2H), 2.19 (t, J=7.0 Hz, 2H), 2.12 (ddd, J=13.9, 10.1, 4.1 Hz, 2H). MS m/z: 475 [M+H]⁺.

### Example 274: 8-[1-(2,2-difluoroethyl)-1H-pyrazolo[3,4-b]pyrazin-6-yl]-2-[6-(trifluoromethyl)pyrazin-2-yl]-2,8-diazaspiro[4.5]decan-3-one

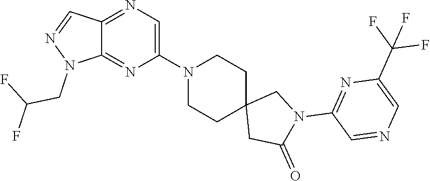 [SMILES: O=C1CC2(CCN(c3cnc4cnn(CC(F)F)c4n3)CC2)CN1c1cncc(C(F)(F)F)n1]

Followed the General Procedure A to afford the desired product as a yellow oil (20 mg, 70%). ¹H NMR (500 MHz, CDCl₃) δ 9.98 (s, 1H), 8.69 (s, 1H), 8.27 (s, 1H), 8.06 (s, 1H), 6.22 (tt, J=55.6, 4.4 Hz, 1H), 4.67 (td, J=13.3, 4.5 Hz, 2H), 3.99 (s, 4H), 3.72 (ddd, J=13.1, 7.7, 4.1 Hz, 2H), 2.73 (s, 2H), 1.89 (dt, J=10.2, 5.0 Hz, 4H). MS m/z: 483 [M+H]⁺.

### Example 275: 8-(1-(2,2-difluoroethyl)-1H-pyrazolo[3,4-b]pyrazin-6-yl)-2-(4-(trifluoromethyl)pyrimidin-2-yl)-2,8-diazaspiro[4.5]decan-3-one

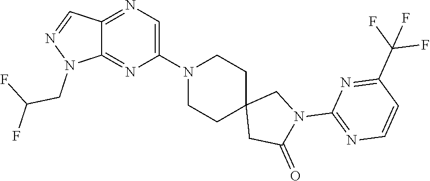 [SMILES: O=C1CC2(CCN(c3cnc4cnn(CC(F)F)c4n3)CC2)CN1c1nccc(C(F)(F)F)n1]

Followed the General procedure A to afford the desired product as a colorless oil (14 mg, 49%). ¹H NMR (500 MHz, CDCl₃) δ 9.00 (d, J=4.9 Hz, 1H), 8.27 (s, 1H), 8.06 (s, 1H), 7.39 (d, J=4.9 Hz, 1H), 6.22 (tt, J=55.6, 4.5 Hz, 1H), 4.67 (td, J=13.3, 4.5 Hz, 2H), 4.02 (s, 2H), 4.00-3.95 (m, 2H), 3.70 (ddd, J=13.9, 7.4, 4.6 Hz, 2H), 2.74 (s, 2H), 1.88 (dq, J=4.7, 3.0, 2.3 Hz, 4H). MS m/z: 483 [M+H]⁺.

### Example 276: 8-[1-(oxetan-3-yl)-1H-pyrazolo[3,4-b]pyrazin-6-yl]-2-[6-(trifluoromethyl)pyrazin-2-yl]-2,8-diazaspiro[4.5]decan-1-one

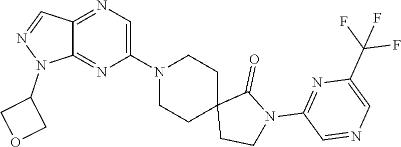 [SMILES: O=C1N(c2cncc(C(F)(F)F)n2)CCC12CCN(c1cnc3cnn(C4COC4)c3n1)CC2]

Followed the General procedure A to afford the desired product as a light yellow oil (20 mg, 71%). ¹H NMR (500 MHz, CDCl₃) δ 9.97 (s, 1H), 8.69 (s, 1H), 8.26 (s, 1H), 8.10 (s, 1H), 5.95 (p, J=7.3 Hz, 1H), 5.34 (t, J=6.6 Hz, 2H), 5.06 (t, J=7.2 Hz, 2H), 4.37 (dt, J=13.9, 4.8 Hz, 2H), 4.11 (t, J=7.1 Hz, 2H), 3.49 (ddd, J=13.7, 10.1, 3.2 Hz, 2H), 2.26 (t, J=7.1 Hz, 2H), 2.13 (ddd, J=14.1, 10.2, 4.1 Hz, 2H), 1.75 (dt, J=14.0, 4.0 Hz, 2H) MS m/z: 475 [M+H]⁺.

### Example 277: 8-[1-(oxetan-3-yl)-1H-pyrazolo[3,4-b]pyrazin-6-yl]-2-[6-(trifluoromethyl)pyrazin-2-yl]-2,8-diazaspiro[4.5]decan-3-one

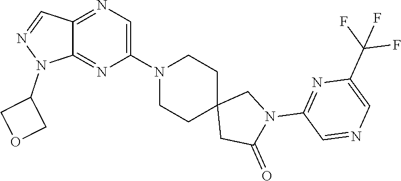 [SMILES: O=C1CC2(CCN(c3cnc4cnn(C5COC5)c4n3)CC2)CN1c1cncc(C(F)(F)F)n1]

Followed the General procedure A to afford the desired product as a yellow oil (18 mg, 64%). ¹H NMR (500 MHz, CDCl₃) δ 9.97 (s, 1H), 8.69 (s, 1H), 8.26 (s, 1H), 8.10 (s, 1H), 5.98-5.90 (m, 1H), 5.33 (t, J=6.6 Hz, 2H), 5.06 (dd, J=7.9, 6.5 Hz, 2H), 3.98 (s, 2H), 3.97-3.93 (m, 2H), 3.71 (ddd, J=13.8, 7.7, 4.1 Hz, 2H), 2.73 (s, 2H), 1.88 (td, J=6.8, 6.2, 4.2 Hz, 4H) MS m/z: 475 [M+H]⁺.

### Example 278: 8-[1-(oxetan-3-yl)-1H-pyrazolo[3,4-b]pyrazin-6-yl]-2-[4-(trifluoromethyl)pyrimidin-2-yl]-2,8-diazaspiro[4.5]decan-1-one

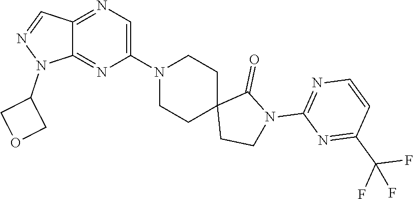 [SMILES: O=C1N(c2nccc(C(F)(F)F)n2)CCC12CCN(c1cnc3cnn(C4COC4)c3n1)CC2]

Followed the General procedure A to afford the desired product as a yellow oil (18 mg, 57%). ¹H NMR (500 MHz, CDCl₃) δ 8.98 (d, J=4.9 Hz, 1H), 8.24 (s, 1H), 8.09 (s, 1H), 7.38 (d, J=4.9 Hz, 1H), 5.94 (tt, J=7.9, 6.6 Hz, 1H), 5.34 (t, J=6.6 Hz, 2H), 5.08-5.03 (m, 2H), 4.32 (ddd, J=13.6, 6.2, 4.0 Hz, 2H), 4.13 (dd, J=7.8, 6.3 Hz, 2H), 3.58 (ddd, J=13.3, 9.4, 3.4 Hz, 2H), 2.21-2.12 (m, 4H), 1.78-1.72 (m, 2H). MS m/z: 475 [M+H]⁺.

### Example 279: 8-[1-(oxetan-3-yl)-1H-pyrazolo[3,4-b]pyrazin-6-yl]-2-[4-(trifluoromethyl)pyrimidin-2-yl]-2,8-diazaspiro[4.5]decan-3-one

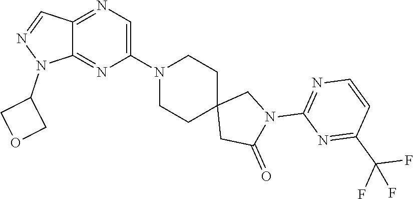 [SMILES: O=C1CC2(CCN(c3cnc4cnn(C5COC5)c4n3)CC2)CN1c1nccc(C(F)(F)F)n1]

Followed the General procedure A to afford the desired product as a colorless oil (16 mg, 57%). ¹H NMR (500 MHz, CDCl₃) δ 9.00 (d, J=4.9 Hz, 1H), 8.25 (s, 1H), 8.10 (s, 1H), 7.39 (d, J=4.9 Hz, 1H), 5.94 (p, J=7.3 Hz, 1H), 5.32 (t, J=6.6 Hz, 2H), 5.06 (t, J=7.2 Hz, 2H), 4.01 (s, 2H), 3.97 (dt, J=13.8, 5.1 Hz, 2H), 3.73-3.62 (m, 2H), 2.73 (s, 2H), 1.88 (q, J=4.9, 4.4 Hz, 4H). MS m/z: 475 [M+H]⁺.

### Example 280: 8-[1-(2,2-difluoroethyl)-3-methyl-1H-pyrazolo[3,4-b]pyrazin-6-yl]-2-[6-(trifluoromethyl)pyrazin-2-yl]-2,8-diazaspiro[4.5]decan-3-one

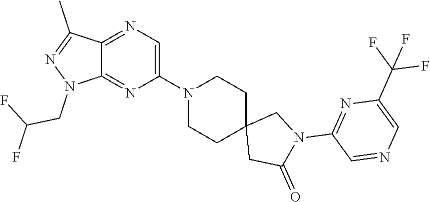 [SMILES: Cc1nn(CC(F)F)c2nc(N3CCC4(CC3)CC(=O)N(c3cncc(C(F)(F)F)n3)C4)cnc12]

Followed the General procedure A to afford the desired product as a colorless oil (23 mg, 78%). ¹H NMR (500 MHz, CDCl₃) δ 9.97 (s, 1H), 8.69 (s, 1H), 8.19 (s, 1H), 6.19 (tt, J=55.7, 4.5 Hz, 1H), 4.59 (td, J=13.4, 4.5 Hz, 2H), 3.98 (s, 2H), 3.97-3.91 (m, 2H), 3.70 (ddd, J=13.9, 7.7, 4.2 Hz, 2H), 2.73 (s, 2H), 2.55 (s, 3H), 1.88 (td, J=6.9, 6.3, 4.1 Hz, 4H). MS m/z: 497 [M+H]⁺.

### Example 281: 8-[1-(2,2-difluoroethyl)-3-methyl-1H-pyrazolo[3,4-b]pyrazin-6-yl]-2-[4-(trifluoromethyl)pyrimidin-2-yl]-2,8-diazaspiro[4.5]decan-1-one

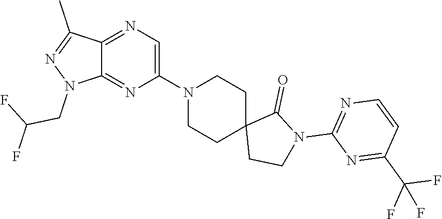 [SMILES: Cc1nn(CC(F)F)c2nc(N3CCC4(CC3)CCN(c3nccc(C(F)(F)F)n3)C4=O)cnc12]

Followed the General procedure A to afford the desired product as a colorless oil (21 mg, 71%). ¹H NMR (500 MHz, CDCl₃) δ 8.98 (d, J=4.9 Hz, 1H), 8.18 (s, 1H), 7.38 (d, J=4.9 Hz, 1H), 6.19 (tt, J=55.8, 4.5 Hz, 1H), 4.59 (td, J=13.4, 4.5 Hz, 2H), 4.31 (ddd, J=13.6, 6.3, 4.1 Hz, 2H), 4.17-4.08 (m, 2H), 3.57 (ddd, J=13.3, 9.4, 3.4 Hz, 2H), 2.55 (s, 3H), 2.23-2.09 (m, 4H), 1.80-1.70 (m, 2H). MS m/z: 497 [M+H]⁺.

### Example 282: 8-[1-(2,2-difluoroethyl)-3-methyl-1H-pyrazolo[3,4-b]pyrazin-6-yl]-2-[4-(trifluoromethyl)pyrimidin-2-yl]-2,8-diazaspiro[4.5]decan-3-one

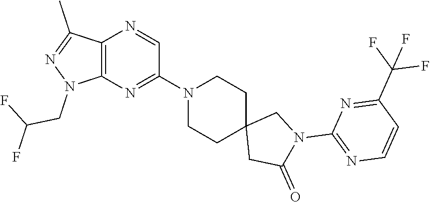 [SMILES: Cc1nn(CC(F)F)c2nc(N3CCC4(CC3)CC(=O)N(c3nccc(C(F)(F)F)n3)C4)cnc12]

Followed the General procedure A to afford the desired product as a colorless oil (18 mg, 61%). ¹H NMR (500 MHz, CDCl₃) δ 9.00 (d, J=4.9 Hz, 1H), 8.19 (s, 1H), 7.39 (d, J=4.9 Hz, 1H), 6.19 (tt, J=55.7, 4.5 Hz, 1H), 4.59 (td, J=13.3, 4.5 Hz, 2H), 4.01 (s, 2H), 4.00-3.92 (m, 2H), 3.68 (ddd, J=13.8, 7.5, 4.6 Hz, 2H), 2.73 (s, 2H), 2.55 (s, 3H), 1.88 (dt, J=6.9, 4.2 Hz, 4H). MS m/z: 497 [M+H]⁺.

### Example 283: 8-[1-(2,2-difluoroethyl)-3-methyl-1H-pyrazolo[3,4-b]pyrazin-6-yl]-2-[4-(trifluoromethyl)pyridin-2-yl]-2,8-diazaspiro[4.5]decan-3-one

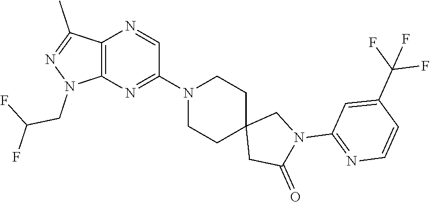 [SMILES: Cc1nn(CC(F)F)c2nc(N3CCC4(CC3)CC(=O)N(c3cc(C(F)(F)F)ccn3)C4)cnc12]

Followed the General procedure A to afford the desired product as a colorless oil (16 mg, 54%). ¹H NMR (500 MHz, CDCl₃) δ 8.76-8.73 (m, 1H), 8.51 (d, J=5.2 Hz, 1H), 8.19 (s, 1H), 7.29-7.27 (m, 1H), 6.19 (tt, J=55.7, 4.5 Hz, 1H), 4.59 (td, J=13.4, 4.5 Hz, 2H), 4.03 (s, 2H), 3.90 (ddd, J=13.8, 6.6, 4.5 Hz, 2H), 3.74 (ddd, J=13.8, 7.0, 4.6 Hz, 2H), 2.70 (s, 2H), 2.55 (s, 3H), 1.86 (dt, J=7.1, 3.8 Hz, 4H). MS m/z: 496 [M+H]⁺.

### Example 284: 8-[1-(oxetan-3-yl)-1H-pyrazolo[3,4-b]pyrazin-6-yl]-2-[4-(trifluoromethyl)pyridin-2-yl]-2,8-diazaspiro[4.5]decan-3-one

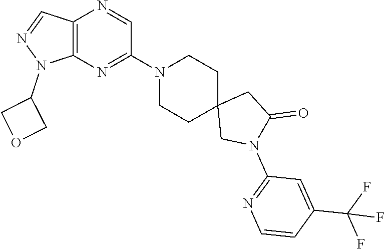 [SMILES: O=C1CC2(CCN(c3cnc4cnn(C5COC5)c4n3)CC2)CN1c1cc(C(F)(F)F)ccn1]

Followed the General procedure A to afford the desired product as a colorless oil (14 mg, 50%). ¹H NMR (500 MHz, CDCl₃) δ 8.75 (s, 1H), 8.51 (d, J=5.2 Hz, 1H), 8.25 (s, 1H), 8.10 (s, 1H), 7.28 (dd, J=5.3, 1.7 Hz, 7H), 5.94 (tt, J=8.0, 6.7 Hz, 1H), 5.33 (t, J=6.6 Hz, 2H), 5.06 (dd, J=7.9, 6.6 Hz, 2H), 4.04 (s, 2H), 3.91 (ddd, J=13.8, 6.6, 4.6 Hz, 2H), 3.75 (ddd, J=13.8, 7.1, 4.6 Hz, 2H), 2.70 (s, 2H), 1.89-1.82 (m, 4H). MS m/z: 474 [M+H]⁺.

### Example 285: 8-[1-(2,2-difluoroethyl)-1H-pyrazolo[3,4-d]pyrimidin-6-yl]-2-[4-(trifluoromethyl)pyridin-2-yl]-2,8-diazaspiro[4.5]decan-1-one

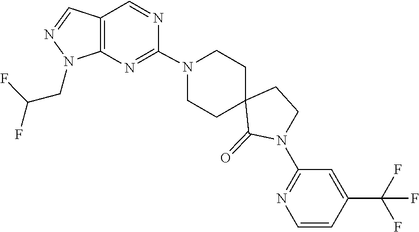 [SMILES: O=C1N(c2cc(C(F)(F)F)ccn2)CCC12CCN(c1ncc3cnn(CC(F)F)c3n1)CC2]

Followed the General procedure A to afford the desired product as a colorless oil (12 mg, 42%). ¹H NMR (500 MHz, CDCl₃) δ 8.77 (d, J=1.5 Hz, 2H), 8.53 (d, J=5.2 Hz, 1H), 7.89 (s, 1H), 7.25 (d, J=1.7 Hz, 1H), 6.22 (tt, J=55.6, 4.5 Hz, 1H), 4.71 (dt, J=13.8, 4.5 Hz, 2H), 4.63 (td, J=13.3, 4.5 Hz, 2H), 4.17-4.06 (m, 2H), 3.45 (ddd, J=13.8, 10.7, 3.2 Hz, 2H), 2.24-2.16 (m, 2H), 2.05 (ddd, J=13.5, 10.7, 4.3 Hz, 2H), 1.67 (dtd, J=13.3, 3.3, 1.6 Hz, 2H). MS m/z: 482 [M+H]⁺.

### Example 286: 8-[1-(2,2-difluoroethyl)-1H-pyrazolo[3,4-d]pyrimidin-6-yl]-2-[4-(trifluoromethyl)pyridin-2-yl]-2,8-diazaspiro[4.5]decan-3-one

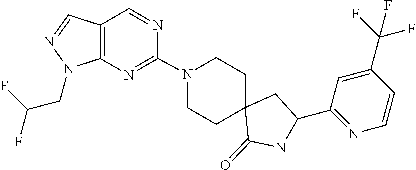 [SMILES: O=C1NC(c2cc(C(F)(F)F)ccn2)CC12CCN(c1ncc3cnn(CC(F)F)c3n1)CC2]

Followed the General procedure A to afford the desired product as a colorless oil (11 mg, 39%). ¹H NMR (500 MHz, CDCl₃) δ 8.76 (d, J=3.7 Hz, 2H), 8.51 (d, J=5.2 Hz, 1H), 7.89 (s, 1H), 7.27 (s, 1H), 6.21 (tt, J=55.6, 4.5 Hz, 1H), 4.63 (td, J=13.3, 4.5 Hz, 2H), 4.17-4.06 (m, 2H), 4.02 (s, 2H), 3.90 (ddd, J=13.8, 6.9, 4.7 Hz, 2H), 2.69 (s, 2H), 1.85-1.73 (m, 4H). MS m/z: 482 [M+H]⁺.

### Example 287: 8-[1-(2,2-difluoroethyl)-1H-pyrazolo[3,4-d]pyrimidin-6-yl]-2-[6-(trifluoromethyl)pyrazin-2-yl]-2,8-diazaspiro[4.5]decan-1-one

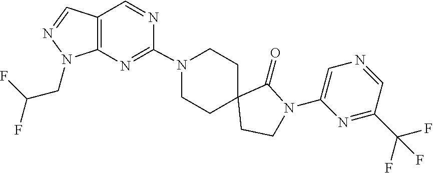 [SMILES: O=C1N(c2cncc(C(F)(F)F)n2)CCC12CCN(c1ncc3cnn(CC(F)F)c3n1)CC2]

Followed the General procedure A to afford the desired product as a colorless oil (11 mg, 39%). ¹H NMR (500 MHz, CDCl₃) δ 9.97 (s, 1H), 8.77 (s, 1H), 8.67 (s, 1H), 7.89 (s, 1H), 6.21 (tt, J=55.6, 4.5 Hz, 1H), 4.73 (dt, J=13.8, 4.5 Hz, 2H), 4.63 (td, J=13.3, 4.5 Hz, 2H), 4.14-4.04 (m, 2H), 3.45 (ddd, J=13.9, 10.8, 3.1 Hz, 2H), 2.26 (t, J=7.1 Hz, 2H), 2.06 (ddd, J=13.6, 10.7, 4.3 Hz, 2H), 1.68 (d, J=13.5 Hz, 2H). MS m/z: 483 [M+H]⁺.

### Example 288: 8-[1-(2,2-difluoroethyl)-1H-pyrazolo[3,4-d]pyrimidin-6-yl]-2-[6-(trifluoromethyl)pyrazin-2-yl]-2,8-diazaspiro[4.5]decan-3-one

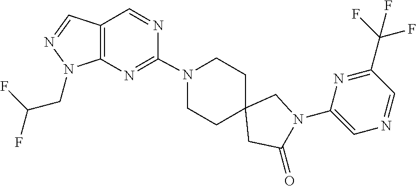 [SMILES: O=C1CC2(CCN(c3ncc4cnn(CC(F)F)c4n3)CC2)CN1c1cncc(C(F)(F)F)n1]

Followed the General procedure A to afford the desired product as a colorless oil (9 mg, 31%). ¹H NMR (500 MHz, CDCl₃) δ 9.98 (s, 1H), 8.77 (s, 1H), 8.68 (s, 1H), 7.89 (s, 1H), 6.21 (tt, J=55.6, 4.5 Hz, 1H), 4.63 (td, J=13.4, 4.5 Hz, 2H), 4.18 (ddd, J=13.9, 6.4, 4.4 Hz, 2H), 3.97 (s, 2H), 3.84 (ddd, J=13.7, 7.6, 4.3 Hz, 2H), 2.72 (s, 2H), 1.85-1.77 (m, 4H). MS m/z: 483 [M+H]⁺.

### Example 289: 8-[1-(2,2-difluoroethyl)-1H-pyrazolo[3,4-d]pyrimidin-6-yl]-2-[4-(trifluoromethyl)pyrimidin-2-yl]-2,8-diazaspiro[4.5]decan-1-one

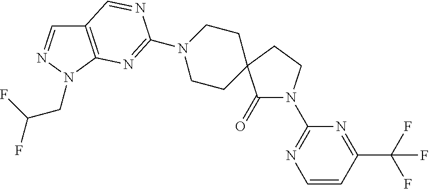 [SMILES: O=C1N(c2nccc(C(F)(F)F)n2)CCC12CCN(c1ncc3cnn(CC(F)F)c3n1)CC2]

Followed the General procedure A to afford the desired product as a colorless oil (11 mg, 38%). ¹H NMR (500 MHz, CDCl₃) δ 8.97 (d, J=4.9 Hz, 1H), 8.76 (s, 1H), 7.88 (s, 1H), 7.37 (d, J=4.9 Hz, 1H), 6.21 (tt, J=55.6, 4.5 Hz, 1H), 4.65 (ddd, J=13.0, 8.3, 4.7 Hz, 4H), 4.12 (t, J=7.0 Hz, 2H), 3.54 (ddd, J=13.6, 10.1, 3.3 Hz, 2H), 2.20 (t, J=7.1 Hz, 2H), 2.09 (ddd, J=14.0, 10.1, 4.2 Hz, 2H), 1.73-1.65 (m, 2H). MS m/z: 483 [M+H]⁺.

### Example 290: 8-[1-(2,2-difluoroethyl)-1H-pyrazolo[3,4-d]pyrimidin-6-yl]-2-[4-(trifluoromethyl)pyrimidin-2-yl]-2,8-diazaspiro[4.5]decan-3-one

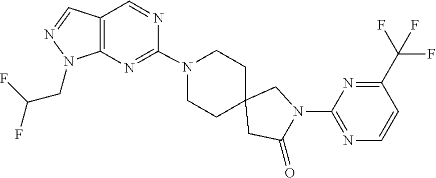 [SMILES: O=C1CC2(CCN(c3ncc4cnn(CC(F)F)c4n3)CC2)CN1c1nccc(C(F)(F)F)n1]

Followed the General procedure A to afford the desired product as a colorless oil (9 mg, 31%). ¹H NMR (500 MHz, CDCl₃) δ 9.00 (d, J=4.9 Hz, 1H), 8.76 (s, 1H), 7.89 (s, 1H), 7.38 (d, J=4.9 Hz, 1H), 6.21 (tt, J=55.6, 4.5 Hz, 1H), 4.63 (td, J=13.3, 4.5 Hz, 2H), 4.19 (dt, J=13.9, 5.3 Hz, 2H), 4.00 (s, 2H), 3.82 (ddd, J=13.9, 7.1, 4.9 Hz, 2H), 2.72 (s, 2H), 1.85-1.76 (m, 4H). MS m/z: 483 [M+H]⁺.

### Example 291: 8-[1-(2,2-difluoroethyl)-1H-pyrazolo[3,4-b]pyrazin-6-yl]-3-phenyl-2-[6-(trifluoromethyl)pyridin-3-yl]-2,8-diazaspiro[4.5]decan-1-one

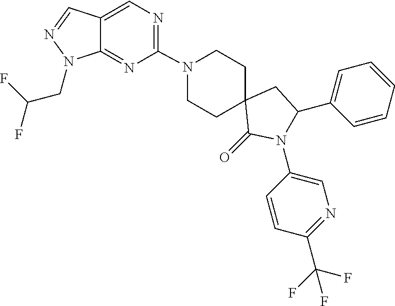 [SMILES: O=C1N(c2ccc(C(F)(F)F)nc2)C(c2ccccc2)CC12CCN(c1ncc3cnn(CC(F)F)c3n1)CC2]

Followed the General procedure A to afford the desired product as a colorless oil (18 mg, 66%). MS m/z: 558 [M+H]⁺.

### Example 292: 8-[1-(2,2-difluoroethyl)-1H-pyrazolo[3,4-b]pyrazin-6-yl]-3-phenyl-2-[5-(trifluoromethyl)pyridin-2-yl]-2,8-diazaspiro[4.5]decan-1-one

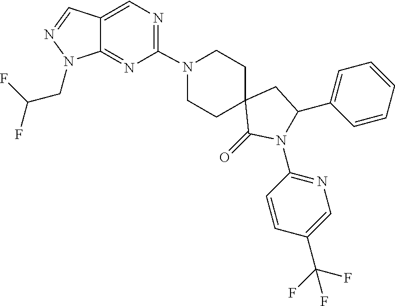 [SMILES: O=C1N(c2ccc(C(F)(F)F)cn2)C(c2ccccc2)CC12CCN(c1ncc3cnn(CC(F)F)c3n1)CC2]

Followed the General procedure A to afford the desired product as a colorless oil (13 mg, 64%). MS m/z: 558 [M+H]⁺.

### Example 293: 9-[1-(2,2-difluoroethyl)-1H-pyrazolo[3,4-b]pyrazin-6-yl]-3-[5-(trifluoromethyl)pyridin-2-yl]-3,9-diazaspiro[5.5]undecan-2-one

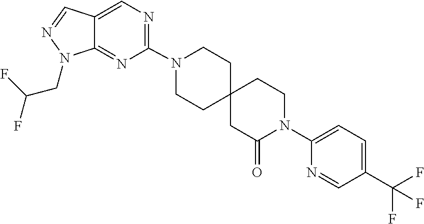 [SMILES: O=C1CC2(CCN(c3ncc4cnn(CC(F)F)c4n3)CC2)CCN1c1ccc(C(F)(F)F)cn1]

Followed the General procedure A to afford the desired product as a colorless oil (21 mg, 59%). ¹H NMR (500 MHz, CDCl₃) δ 8.72-8.63 (m, 1H), 8.24 (s, 1H), 8.14 (d, J=8.7 Hz, 1H), 8.04 (d, J=1.0 Hz, 1H), 7.90 (dd, J=8.8, 2.5 Hz, 1H), 6.21 (tt, J=55.6, 4.5 Hz, 1H), 4.65 (td, J=13.4, 4.5 Hz, 2H), 4.10 (dd, J=7.1, 5.5 Hz, 2H), 3.81 (dtd, J=25.6, 13.5, 5.7 Hz, 4H), 2.63 (s, 2H), 1.97 (t, J=6.4 Hz, 2H), 1.73 (t, J=5.7 Hz, 4H). MS m/z: 496 [M+H]⁺.

### Example 294: 9-[1-(2,2-difluoroethyl)-1H-pyrazolo[3,4-b]pyrazin-6-yl]-2-[5-(trifluoromethyl)pyridin-2-yl]-2,9-diazaspiro[5.5]undecan-3-one

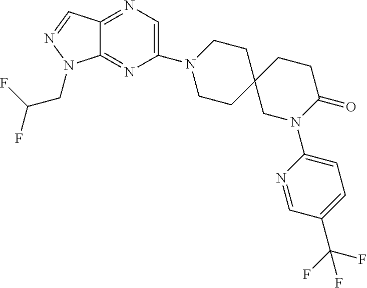 [SMILES: O=C1CCC2(CCN(c3cnc4cnn(CC(F)F)c4n3)CC2)CN1c1ccc(C(F)(F)F)cn1]

Followed the General procedure A to afford the desired product as a colorless oil (14 mg, 40%). ¹H NMR (500 MHz, CDCl₃) δ 8.64 (d, J=2.4 Hz, 1H), 8.23 (s, 1H), 8.18 (d, J=8.8 Hz, 1H), 8.04 (s, 1H), 7.90 (dd, J=8.8, 2.5 Hz, 1H), 6.21 (tt, J=55.6, 4.5 Hz, 1H), 4.65 (td, J=13.3, 4.5 Hz, 2H), 4.04 (s, 2H), 3.87 (ddd, J=13.9, 7.3, 4.2 Hz, 2H), 3.76 (ddd, J=13.8, 7.6, 4.2 Hz, 2H), 2.71 (t, J=7.2 Hz, 2H), 1.91 (t, J=7.2 Hz, 2H), 1.75 (qdt, J=11.3, 7.2, 4.1 Hz, 4H). MS m/z: 496 [M+H]⁺.

### Example 295: 6-[1-(2,2-difluoroethyl)-1H-pyrazolo[3,4-d]pyrimidin-6-yl]-2-[2-(trifluoromethyl)pyridin-4-yl]-2,6-diazaspiro[3.4]octane

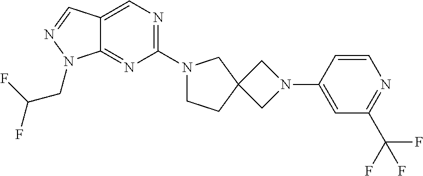 [SMILES: FC(F)Cn1ncc2cnc(N3CCC4(CN(c5ccnc(C(F)(F)F)c5)C4)C3)nc21]

Followed the General procedure A to afford the desired product as an off-white solid (12.6 mg, 30%). ¹H NMR (500 MHz, CDCl₃) δ 8.78 (s, 1H), 8.31 (d, J=5.6 Hz, 1H), 7.90 (d, J=1.3 Hz, 1H), 6.63 (d, J=2.3 Hz, 1H), 6.38 (dd, J=5.9, 2.2 Hz, 1H), 6.22 (tt, J=55.6, 4.6 Hz, 1H), 4.64 (td, J=13.4, 4.4 Hz, 2H), 4.09-3.97 (m, 4H), 3.92 (s, 2H), 3.79 (s, 2H), 2.33 (t, J=6.9 Hz, 2H). MS m/z: 440 [M+H]⁺.

### Example 296: 6-[1-(2,2-difluoroethyl)-3-methyl-1H-pyrazolo[3,4-d]pyrimidin-6-yl]-2-[2-(trifluoromethyl)pyridin-4-yl]-2,6-diazaspiro[3.4]octane

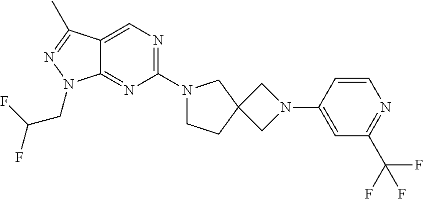 [SMILES: Cc1nn(CC(F)F)c2nc(N3CCC4(CN(c5ccnc(C(F)(F)F)c5)C4)C3)ncc12]

Followed the General procedure A to afford the desired product as a colorless oil (22 mg, 71%). ¹H NMR (500 MHz, CDCl₃) δ 8.68 (s, 1H), 8.32 (d, J=5.6 Hz, 1H), 6.62 (d, J=2.2 Hz, 1H), 6.38 (dd, J=5.7, 2.3 Hz, 1H), 6.19 (tt, J=55.7, 4.5 Hz, 1H), 4.55 (td, J=13.4, 4.5 Hz, 2H), 4.09-3.97 (m, 4H), 3.92 (s, 2H), 3.78 (s, 2H), 2.49 (s, 3H), 2.33 (t, J=6.9 Hz, 2H). MS m/z: 454 [M+H]⁺.

### Example 297: 6-[1-(2,2-difluoroethyl)-4-methoxy-1H-pyrazolo[3,4-d]pyrimidin-6-yl]-2-[2-(trifluoromethyl)pyridin-4-yl]-2,6-diazaspiro[3.4]octane

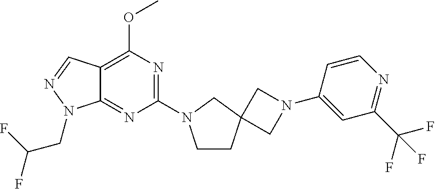 [SMILES: COc1nc(N2CCC3(CN(c4ccnc(C(F)(F)F)c4)C3)C2)nc2c1cnn2CC(F)F]

Followed the General procedure A to afford the desired product as a colorless oil (16 mg, 50%). ¹H NMR (500 MHz, CDCl₃) δ 8.32 (d, J=5.6 Hz, 1H), 7.82 (s, 1H), 6.62 (d, J=2.2 Hz, 1H), 6.38 (dd, J=5.8, 2.3 Hz, 1H), 6.21 (tt, J=55.8, 4.6 Hz, 1H), 4.59 (td, J=13.3, 4.5 Hz, 2H), 4.05 (d, J=4.6 Hz, 5H), 4.00 (d, J=7.9 Hz, 2H), 3.90 (s, 2H), 3.76 (s, 2H), 2.30 (t, J=6.9 Hz, 2H). MS m/z: 470 [M+H]⁺.

### Example 298: 6-[1-(2,2-difluoroethyl)-1H-pyrazolo[3,4-b]pyrazin-6-yl]-2-[6-(trifluoromethyl)pyrazin-2-yl]-2,6-diazaspiro[3.4]octane

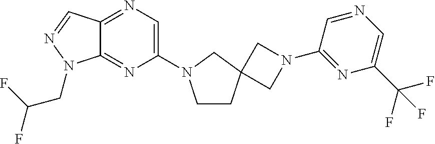 [SMILES: FC(F)Cn1ncc2ncc(N3CCC4(CN(c5cncc(C(F)(F)F)n5)C4)C3)nc21]

Followed the General procedure A to afford the desired product as a colorless oil (2.4 mg, 8%). ¹H NMR (500 MHz, CDCl₃) δ 8.18 (s, 1H), 8.16 (s, 1H), 8.05 (d, J=1.2 Hz, 1H), 7.96-7.91 (m, 1H), 6.35-6.08 (m, 1H), 5.27 (q, J=7.6 Hz, 1H), 4.66 (td, J=13.4, 4.5 Hz, 2H), 3.89 (ddd, J=11.8, 8.5, 6.0 Hz, 2H), 3.75-3.69 (m, 4H), 3.60 (d, J=11.7 Hz, 1H), 2.12 (dtd, J=17.7, 13.2, 6.6 Hz, 2H). MS m/z: 441 [M+H]⁺.

### Example 299: 6-[1-(2,2-difluoroethyl)-1H-pyrazolo[3,4-b]pyrazin-6-yl]-2-[4-(trifluoromethyl)pyrimidin-2-yl]-2,6-diazaspiro[3.4]octane

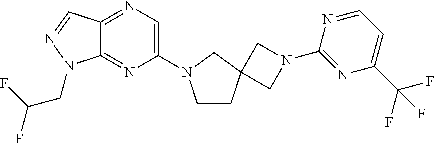 [SMILES: FC(F)Cn1ncc2ncc(N3CCC4(C3)CN(c3nccc(C(F)(F)F)n3)C4)nc21]

Followed the General procedure A to afford the desired product as a colorless oil (17 mg, 57%). ¹H NMR (500 MHz, CDCl₃) δ 8.57-8.47 (m, 1H), 8.05 (d, J=1.6 Hz, 1H), 7.97 (d, J=1.6 Hz, 1H), 6.86 (dd, J=5.0, 1.7 Hz, 1H), 6.22 (tdd, J=55.6, 5.7, 2.9 Hz, 1H), 4.72-4.59 (m, 2H), 4.29-4.14 (m, 4H), 3.88 (s, 2H), 3.74 (t, J=6.9 Hz, 2H), 2.38 (t, J=6.9 Hz, 2H). MS m/z: 441 [M+H]⁺.

### Example 300: 2-[1-(2,2-difluoroethyl)-1H-pyrazolo[3,4-b]pyrazin-6-yl]-5-(propan-2-yl)-7-[4-(trifluoromethyl)pyridin-2-yl]-2,5,7-triazaspiro[3.4]octane-6,8-dione

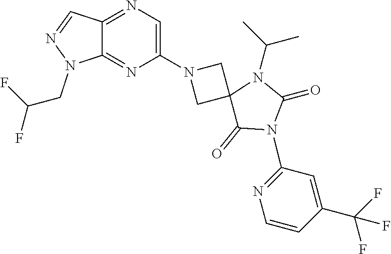 [SMILES: CC(C)N1C(=O)N(c2cc(C(F)(F)F)ccn2)C(=O)C12CN(c1cnc3cnn(CC(F)F)c3n1)C2]

Followed the General procedure A to afford the desired product as a colorless oil (2.6 mg, 12%). MS m/z: 511 [M+H]⁺.

### Example 301: 8-[1-(2,2-difluoroethyl)-3-methyl-1H-pyrazolo[3,4-d]pyrimidin-6-yl]-2-[4-(trifluoromethyl)pyridin-2-yl]-2,8-diazaspiro[4.5]decan-1-one

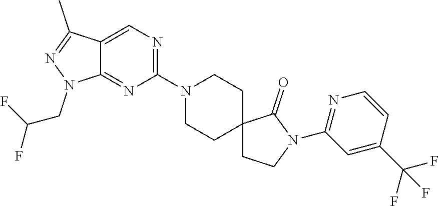 [SMILES: Cc1nn(CC(F)F)c2nc(N3CCC4(CC3)CCN(c3cc(C(F)(F)F)ccn3)C4=O)ncc12]

Followed the General procedure A to afford the desired product as a colorless oil (20 mg, 68%). ¹H NMR (500 MHz, CDCl₃) δ 8.76 (s, 1H), 8.67 (s, 1H), 8.52 (d, J=5.1 Hz, 1H), 7.25 (s, 1H), 6.19 (ddt, J=60.2, 55.8, 3.8 Hz, 1H), 4.78-4.67 (m, 2H), 4.59-4.50 (m, 2H), 4.11 (t, J=7.2 Hz, 2H), 3.42 (t, J=12.3 Hz, 2H), 2.48 (s, 3H), 2.20 (t, J=7.2 Hz, 2H), 2.08-1.99 (m, 2H). MS m/z: 496 [M+H]⁺.

### Example 302: 8-[1-(2,2-difluoroethyl)-3-methyl-1H-pyrazolo[3,4-d]pyrimidin-6-yl]-2-[4-(trifluoromethyl)pyridin-2-yl]-2,8-diazaspiro[4.5]decan-3-one

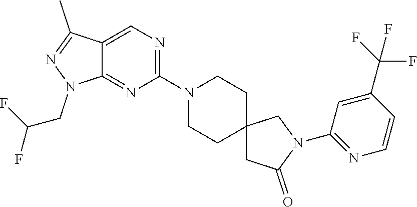 [SMILES: Cc1nn(CC(F)F)c2nc(N3CCC4(CC3)CC(=O)N(c3cc(C(F)(F)F)ccn3)C4)ncc12]

Followed the General procedure A to afford the desired product as a colorless oil (22 mg, 75%). ¹H NMR (500 MHz, CDCl₃) δ 8.75 (dt, J=1.6, 0.9 Hz, 1H), 8.66 (s, 1H), 8.51 (d, J=5.2 Hz, 1H), 7.27 (d, J=1.6 Hz, 1H), 6.18 (tt, J=55.7, 4.5 Hz, 1H), 4.55 (td, J=13.4, 4.5 Hz, 2H), 4.17-4.06 (m, 2H), 4.02 (s, 2H), 3.88 (ddd, J=13.8, 6.9, 4.7 Hz, 2H), 2.69 (s, 2H), 2.48 (s, 3H), 1.78 (ddd, J=6.7, 4.5, 2.4 Hz, 4H). MS m/z: 496 [M+H]⁺.

### Example 303: 8-[1-(2,2-difluoroethyl)-4-methoxy-1H-pyrazolo[3,4-d]pyrimidin-6-yl]-2-[4-(trifluoromethyl)pyridin-2-yl]-2,8-diazaspiro[4.5]decan-1-one

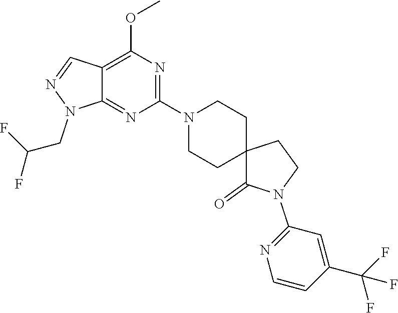 [SMILES: COc1nc(N2CCC3(CC2)CCN(c2cc(C(F)(F)F)ccn2)C3=O)nc2c1cnn2CC(F)F]

Followed the General procedure A to afford the desired product as a colorless oil (22 mg, 72%). ¹H NMR (500 MHz, CDCl₃) δ 8.77 (dd, J=1.6, 0.9 Hz, 1H), 8.53 (d, J=5.2 Hz, 1H), 7.81 (s, 1H), 7.26 (d, J=6.2 Hz, 1H, overlapped with CDCl₃ peak), 6.20 (tt, J=55.8, 4.6 Hz, 1H), 4.68 (dt, J=13.7, 4.5 Hz, 2H), 4.60 (td, J=13.3, 4.5 Hz, 2H), 4.13-4.09 (m, 2H), 4.04 (s, 3H), 3.40 (ddd, J=13.8, 10.8, 3.1 Hz, 2H), 2.20 (t, J=7.1 Hz, 2H), 2.03 (ddd, J=13.5, 10.8, 4.3 Hz, 2H), 1.65 (dt, J=13.8, 3.6 Hz, 2H). MS m/z: 512 [M+H]⁺.

### Example 304: 8-[1-(2,2-difluoroethyl)-4-methoxy-1H-pyrazolo[3,4-d]pyrimidin-6-yl]-2-[4-(trifluoromethyl)pyridin-2-yl]-2,8-diazaspiro[4.5]decan-3-one

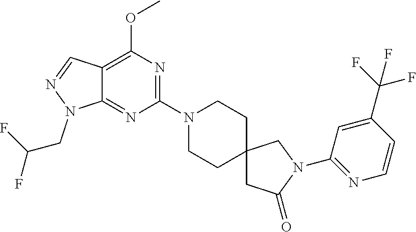 [SMILES: COc1nc(N2CCC3(CC2)CC(=O)N(c2cc(C(F)(F)F)ccn2)C3)nc2c1cnn2CC(F)F]

Followed the General procedure A to afford the desired product as a colorless oil (23 mg, 75%). ¹H NMR (500 MHz, CDCl₃) δ 8.73 (dt, J=1.7, 0.8 Hz, 1H), 8.49 (dt, J=5.3, 0.7 Hz, 1H), 7.79 (s, 1H), 7.26-7.24 (m, 1H), 6.18 (tt, J=55.7, 4.5 Hz, 1H), 4.57 (td, J=13.3, 4.5 Hz, 2H), 4.11-4.05 (m, 4H), 4.02 (s, 3H), 4.00 (s, 2H), 3.83 (ddd, J=13.7, 7.0, 4.7 Hz, 2H), 2.67 (s, 2H), 1.75 (td, J=4.6, 1.9 Hz, 4H). MS m/z: 512 [M+H]⁺.

### Example 305: 8-[1-(2,2-difluoroethyl)-1H-pyrazolo[3,4-b]pyrazin-6-yl]-2-[2-(trifluoromethyl)pyrimidin-5-yl]-2,8-diazaspiro[4.5]decan-3-one

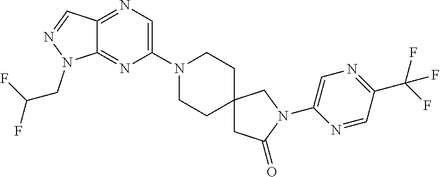 [SMILES: O=C1CC2(CCN(c3cnc4cnn(CC(F)F)c4n3)CC2)CN1c1cnc(C(F)(F)F)cn1]

Followed the General procedure A to afford the desired product as a colorless oil (5 mg, 96%). ¹H NMR (500 MHz, CDCl₃) δ 9.26 (s, 2H), 8.28 (s, 1H), 8.07 (s, 1H), 6.21 (tt, J=55.6, 4.5 Hz, 1H), 4.67 (td, J=13.3, 4.5 Hz, 2H), 4.08 (dt, J=14.0, 5.0 Hz, 2H), 3.78 (s, 2H), 3.69-3.55 (m, 2H), 2.72 (s, 2H), 1.91 (dd, J=6.8, 4.6 Hz, 4H). MS m/z: 483 [M+H]⁺.

### Example 306: 8-[1-(2,2-difluoroethyl)-1H-pyrazolo[3,4-b]pyrazin-6-yl]-2-[2-(trifluoromethyl)pyrimidin-5-yl]-2,8-diazaspiro[4.5]decan-1-one

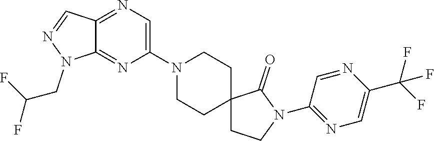 [SMILES: O=C1N(c2cnc(C(F)(F)F)cn2)CCC12CCN(c1cnc3cnn(CC(F)F)c3n1)CC2]

Followed the General procedure A to afford the desired product as a white solid (12 mg, 42%). ¹H NMR (500 MHz, CDCl₃) δ 9.31 (s, 2H), 8.27 (s, 1H), 8.06 (s, 1H), 6.22 (tt, J=55.6, 4.5 Hz, 1H), 4.67 (td, J=13.3, 4.5 Hz, 2H), 4.35 (dt, J=13.8, 4.8 Hz, 2H), 3.95 (t, J=7.0 Hz, 2H), 3.53 (ddd, J=13.5, 9.9, 3.3 Hz, 2H), 2.33 (t, J=7.0 Hz, 2H), 2.13 (ddd, J=13.8, 9.9, 4.0 Hz, 2H), 1.78-1.71 (m, 2H). MS m/z: 483 [M+H]⁺.

### Example 307: 8-[1-(2,2-difluoroethyl)-1H-pyrazolo[3,4-b]pyrazin-6-yl]-2-{[6-(trifluoromethyl)pyridin-3-yl]methyl}-2,8-diazaspiro[4.5]decan-3-one

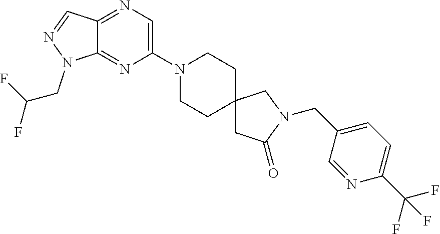 [SMILES: O=C1CC2(CCN(c3cnc4cnn(CC(F)F)c4n3)CC2)CN1Cc1ccc(C(F)(F)F)nc1]

Followed the General procedure A to afford the desired product as a colorless oil (16 mg, 54%). ¹H NMR (500 MHz, CDCl₃) δ 8.63 (d, J=2.1 Hz, 1H), 8.22 (s, 1H), 8.04 (s, 1H), 7.82 (dd, J=8.1, 2.2 Hz, 1H), 7.70 (dd, J=8.1, 0.8 Hz, 1H), 6.19 (tt, J=55.6, 4.4 Hz, 1H), 4.64 (td, J=13.4, 4.5 Hz, 2H), 4.58 (s, 2H), 3.89 (ddd, J=13.8, 6.4, 4.1 Hz, 2H), 3.58 (ddd, J=13.9, 8.6, 3.7 Hz, 2H), 3.18 (s, 2H), 2.48 (s, 2H), 1.73 (m, 4H). MS m/z: 496 [M+H]⁺.

### Example 308: 8-[1-(2,2-difluoroethyl)-1H-pyrazolo[3,4-b]pyrazin-6-yl]-2-{[6-(trifluoromethyl)pyridin-3-yl]methyl}-2,8-diazaspiro[4.5]decan-1-one

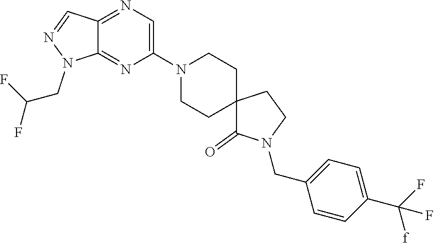

Followed the General procedure A to afford the desired product as a colorless oil (24 mg, 81%). ¹H NMR (500 MHz, CDCl₃) δ 8.62 (d, J=2.1 Hz, 1H), 8.24 (s, 1H), 8.04 (s, 1H), 7.79 (dd, J=8.1, 2.2 Hz, 1H), 7.71-7.67 (m, 1H), 6.21 (tt, J=55.6, 4.5 Hz, 1H), 4.66 (td, J=13.3, 4.5 Hz, 2H), 4.57 (s, 2H), 4.35 (dt, J=13.7, 4.7 Hz, 2H), 3.44 (ddd, J=13.6, 10.2, 3.3 Hz, 2H), 3.30 (t, J=6.9 Hz, 2H), 2.10-2.00 (m, 4H), 1.62-1.56 (m, 2H). MS m/z: 496 [M+H]⁺.

### Example 309: 8-[1-(2,2-difluoroethyl)-1H-pyrazolo[3,4-b]pyrazin-6-yl]-2-{[5-(trifluoromethyl)pyridin-2-yl]methyl}-2,8-diazaspiro[4.5]decan-3-one

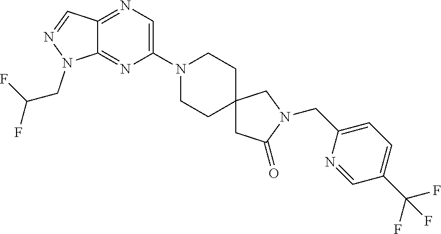 [SMILES: O=C1CC2(CCN(c3cnc4cnn(CC(F)F)c4n3)CC2)CN1Cc1ccc(C(F)(F)F)cn1]

Followed the General procedure A to afford the desired product as a colorless oil (14 mg, 48%). ¹H NMR (500 MHz, CDCl₃) δ 8.82 (s, 1H), 8.23 (s, 1H), 8.04 (s, 1H), 7.93 (dd, J=8.2, 2.3 Hz, 1H), 7.43 (d, J=8.1 Hz, 1H), 6.20 (tt, J=55.6, 4.5 Hz, 1H), 4.73-4.58 (m, 4H), 3.86 (ddd, J=13.8, 6.6, 4.2 Hz, 2H), 3.65 (ddd, J=13.8, 8.0, 4.0 Hz, 2H), 3.35 (s, 2H), 2.48 (s, 2H), 1.77 (qt, J=13.3, 6.7 Hz, 4H). MS m/z: 496 [M+H]⁺.

### Example 310: 8-[1-(2,2-difluoroethyl)-1H-pyrazolo[3,4-b]pyrazin-6-yl]-2-{[5-(trifluoromethyl)pyridin-2-yl]methyl}-2,8-diazaspiro[4.5]decan-1-one

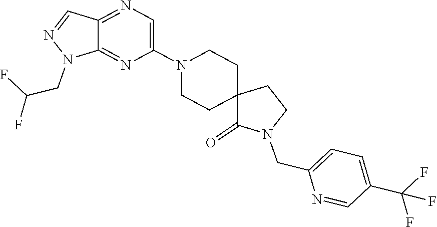 [SMILES: O=C1N(Cc2ccc(C(F)(F)F)cn2)CCC12CCN(c1cnc3cnn(CC(F)F)c3n1)CC2]

Followed the General procedure A to afford the desired product as a colorless oil (11 mg, 37%). ¹H NMR (500 MHz, CDCl₃) δ 8.82 (s, 1H), 8.24 (s, 1H), 8.04 (s, 1H), 7.92 (dd, J=8.2, 2.3 Hz, 1H), 7.38 (d, J=8.1 Hz, 1H), 6.21 (tt, J=55.6, 4.5 Hz, 1H), 4.70-4.62 (m, 4H), 4.35 (dt, J=13.6, 4.8 Hz, 2H), 3.52-3.41 (m, 4H), 2.13-2.02 (m, 4H), 1.67-1.62 (m, 2H). MS m/z: 496 [M+H]⁺.

### Example 311: 8-[1-(2,2-difluoroethyl)-1H-pyrazolo[3,4-b]pyrazin-6-yl]-12-[4-(trifluoromethyl)pyridin-2-yl]-8,12-diazadispiro[2.1.5⁵.2³]dodecan-11-one

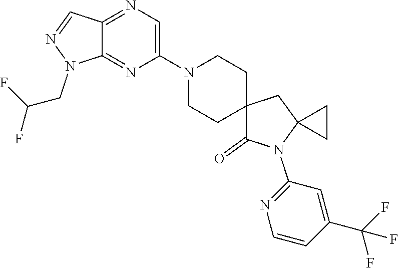 [SMILES: O=C1N(c2cc(C(F)(F)F)ccn2)C2(CC2)CC12CCN(c1cnc3cnn(CC(F)F)c3n1)CC2]

Followed the General procedure A to afford the desired product as a colorless oil (10 mg, 71%). ¹H NMR (500 MHz, CDCl₃) δ 8.51 (d, J=5.1 Hz, 1H), 8.26 (s, 1H), 8.04 (d, J=1.4 Hz, 2H), 7.30 (dd, J=5.1, 1.5 Hz, 1H), 6.22 (tt, J=55.6, 4.5 Hz, 1H), 4.67 (td, J=13.3, 4.5 Hz, 2H), 4.33 (ddd, J=13.6, 6.0, 4.1 Hz, 2H), 3.55 (ddd, J=13.3, 9.6, 3.4 Hz, 2H), 2.25-2.17 (m, 4H), 1.90-1.79 (m, 4H), 0.70-0.63 (m, 2H). MS m/z: 508 [M+H]⁺.

### Example 312: 8-[1-(2,2-difluoroethyl)-1H-pyrazolo[3,4-b]pyrazin-6-yl]-12-[5-(trifluoromethyl)pyridin-2-yl]-8,12-diazadispiro[2.1.5⁵.2³]dodecan-11-one

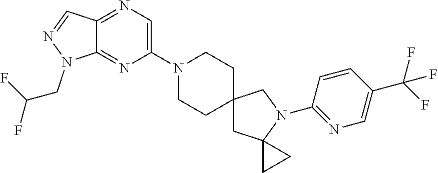 [SMILES: FC(F)Cn1ncc2ncc(N3CCC4(CC3)CN(c3ccc(C(F)(F)F)cn3)C3(CC3)C4)nc21]

Followed the General procedure A to afford the desired product as a colorless oil (5 mg, 56%). ¹H NMR (500 MHz, CDCl₃) δ 8.59 (s, 1H), 8.24 (s, 1H), 8.03 (s, 1H), 7.98-7.83 (m, 2H), 6.20 (tt, J=55.6, 4.5 Hz, 1H), 4.64 (td, J=13.3, 4.5 Hz, 2H), 4.32 (dt, J=13.7, 4.9 Hz, 2H), 3.50 (ddd, J=13.4, 9.8, 3.4 Hz, 2H), 2.23-2.09 (m, 4H), 1.94-1.77 (m, 4H), 0.71-0.60 (m, 2H). MS m/z: 508 [M+H]⁺.

### Example 313: 8-[1-(2,2-difluoroethyl)-1H-pyrazolo[3,4-b]pyrazin-6-yl]-2-[6-(trifluoromethyl)pyridine-3-carbonyl]-2,8-diazaspiro[4.5]decane

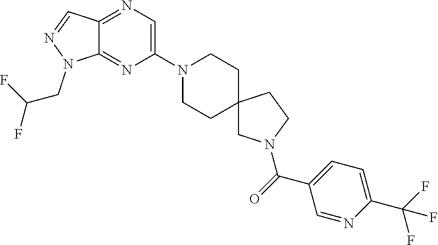 [SMILES: O=C(c1ccc(C(F)(F)F)nc1)N1CCC2(CCN(c3cnc4cnn(CC(F)F)c4n3)CC2)C1]

Followed the General procedure A to afford the desired product as a colorless oil (14 mg, 51%). ¹H NMR (500 MHz, CDCl₃) δ 8.88 (dd, J=8.2, 2.1 Hz, 1H), 8.23 (d, J=24.8 Hz, 1H), 8.10-7.99 (m, 2H), 7.78 (ddd, J=8.1, 2.3, 0.9 Hz, 1H), 6.20 (tdt, J=55.6, 12.9, 4.5 Hz, 1H), 4.65 (qd, J=13.4, 4.5 Hz, 2H), 3.99 (ddd, J=13.7, 6.4, 4.0 Hz, 1H), 3.83 (dd, J=8.0, 6.6 Hz, 1H), 3.75-3.62 (m, 4H), 3.60 (t, J=7.0 Hz, 1H), 3.37 (s, 1H), 2.01 (dd, J=8.1, 6.6 Hz, 1H), 1.93 (t, J=7.0 Hz, 1H), 1.87-1.64 (m, 4H). MS m/z: 496 [M+H]⁺.

### Example 314: 8-[1-(2,2-difluoroethyl)-1H-pyrazolo[3,4-b]pyrazin-6-yl]-2-[5-(trifluoromethyl)pyridine-2-carbonyl]-2,8-diazaspiro[4.5]decane

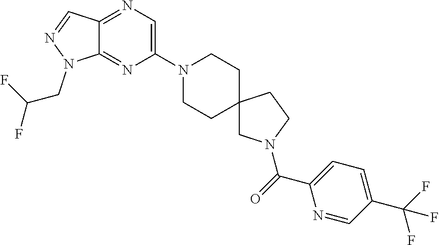 [SMILES: O=C(c1ccc(C(F)(F)F)cn1)N1CCC2(CCN(c3cnc4cnn(CC(F)F)c4n3)CC2)C1]

Followed the General procedure A to afford the desired product as a colorless oil (23 mg, 83%). ¹H NMR (500 MHz, CDCl₃) δ 8.86 (s, 1H), 8.23 (d, J=14.2 Hz, 1H), 8.08-8.00 (m, 3H), 6.21 (tdt, J=55.6, 8.7, 4.5 Hz, 1H), 4.65 (tdd, J=13.8, 9.7, 4.5 Hz, 2H), 4.02-3.93 (m, 2H), 3.87-3.62 (m, 6H), 1.93 (q, J=7.3 Hz, 2H), 1.85-1.69 (m, 4H). MS m/z: 496 [M+H]⁺.

### Example 315: 2-[1-(2,2-difluoroethyl)-1H-pyrazolo[3,4-b]pyrazin-6-yl]-6-[5-(trifluoromethyl)pyridin-2-yl]-2,6-diazaspiro[3.5]nonan-7-one

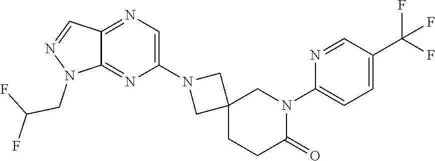 [SMILES: O=C1CCC2(CN(c3cnc4cnn(CC(F)F)c4n3)C2)CN1c1ccc(C(F)(F)F)cn1]

Followed the General procedure A to afford the desired product as a colorless oil (20 mg, 62%). ¹H NMR (500 MHz, CDCl₃) δ 8.66 (dt, J=2.7, 1.0 Hz, 1H), 8.12 (d, J=8.8 Hz, 1H), 8.06 (s, 1H), 7.92 (dd, J=8.8, 2.5 Hz, 1H), 7.81 (s, 1H), 6.21 (tt, J=55.6, 4.4 Hz, 1H), 4.65 (td, J=13.3, 4.5 Hz, 2H), 4.31 (s, 2H), 4.20-4.08 (m, 4H), 2.78 (t, J=7.0 Hz, 2H), 2.27 (t, J=7.0 Hz, 2H). MS m/z: 468 [M+H]⁺.

### Example 316: 6-[1-(2,2-difluoroethyl)-1H-pyrazolo[3,4-b]pyrazin-6-yl]-2-[2-(trifluoromethyl)pyrimidin-5-yl]-2,6-diazaspiro[3.4]octane

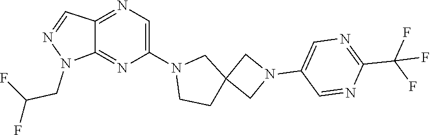

Followed the General procedure A to afford the desired product as a white wax (8 mg, 30.04%). ¹H NMR (500 MHz, CDCl₃) δ 8.05 (s, 1H), 8.01 (s, 2H), 7.95 (s, 1H), 6.20 (tt, J=55.6, 4.5 Hz, 1H), 4.65 (td, J=13.3, 4.5 Hz, 2H), 4.16-4.06 (m, 4H), 3.91 (s, 2H), 3.74 (t, J=6.9 Hz, 2H), 2.42 (t, J=6.9 Hz, 2H). MS m/z: 441 [M+H]⁺.

### Example 317: 6-[1-(2,2-difluoroethyl)-1H-pyrazolo[3,4-b]pyrazin-6-yl]-2-[5-(trifluoromethyl)pyrazin-2-yl]-2,6-diazaspiro[3.4]octane

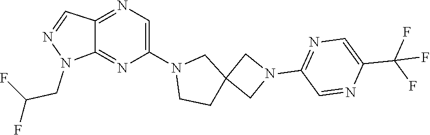

Followed the General procedure A to afford the desired product as a colorless oil (19 mg, 71.35%). ¹H NMR (500 MHz, CDCl₃) δ 8.37 (d, J=1.4 Hz, 1H), 8.06 (s, 1H), 7.97 (s, 1H), 7.81 (d, J=1.4 Hz, 1H), 6.22 (tt, J=55.6, 4.5 Hz, 1H), 4.67 (td, J=13.3, 4.5 Hz, 2H), 4.29-4.18 (m, 4H), 3.92 (s, 2H), 3.76 (t, J=6.9 Hz, 2H), 2.42 (t, J=6.9 Hz, 2H). MS m/z: 441 [M+H]⁺.

### Example 318: 6-[1-(2,2-difluoroethyl)-1H-pyrazolo[3,4-b]pyrazin-6-yl]-2-[2-(trifluoromethyl)pyrimidin-4-yl]-2,6-diazaspiro[3.4]octane

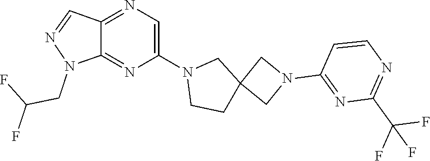

Followed the General procedure A to afford the desired product as a colorless oil (16 mg, 60.09%). ¹H NMR (500 MHz, CDCl₃) δ 8.30 (d, J=5.9 Hz, 1H), 8.06 (s, 1H), 7.97 (s, 1H), 6.31 (d, J=5.9 Hz, 1H), 6.22 (tt, J=55.7, 4.5 Hz, 1H), 4.67 (td, J=13.3, 4.5 Hz, 2H), 4.32-4.09 (m, 4H), 3.90 (s, 2H), 3.75 (t, J=6.9 Hz, 2H), 2.40 (t, J=6.9 Hz, 2H). MS m/z: 441 [M+H]⁺.

### Example 319: 6-[1-(2,2-difluoroethyl)-1H-pyrazolo[3,4-b]pyrazin-6-yl]-2-[5-(trifluoromethyl)pyrimidin-2-yl]-2,6-diazaspiro[3.4]octane

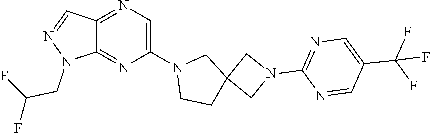 [SMILES: FC(F)Cn1ncc2ncc(N3CCC4(C3)CN(c3ncc(C(F)(F)F)cn3)C4)nc21]

Followed the General procedure A to afford the desired product as a white solid (15 mg, 56.33%). ¹H NMR (500 MHz, CDCl₃) δ 8.55-8.49 (m, 2H), 8.06 (s, 1H), 7.97 (s, 1H), 6.22 (tt, J=55.7, 4.5 Hz, 1H), 4.67 (td, J=13.3, 4.5 Hz, 2H), 4.29-4.18 (m, 4H), 3.90 (s, 2H), 3.75 (t, J=6.9 Hz, 2H), 2.39 (t, J=6.9 Hz, 2H). MS m/z: 441 [M+H]⁺.

### Example 320: 6-[1-(2,2-difluoroethyl)-1H-pyrazolo[3,4-b]pyrazin-6-yl]-2-{[6-(trifluoromethyl)pyridin-3-yl]methyl}-2,6-diazaspiro[3.4]octane

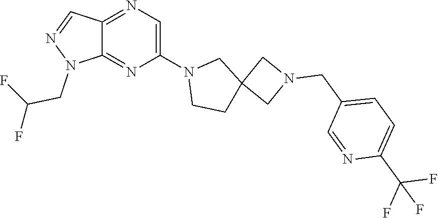 [SMILES: FC(F)Cn1ncc2ncc(N3CCC4(CN(Cc5ccc(C(F)(F)F)nc5)C4)C3)nc21]

Followed the General procedure A to afford the desired product as a colorless oil (12 mg, 43.77%). ¹H NMR (500 MHz, CDCl₃) δ 8.69-8.65 (m, 1H), 8.04 (s, 1H), 7.93 (s, 2H), 7.68 (d, J=8.0 Hz, 1H), 6.22 (tt, J=55.7, 4.5 Hz, 1H), 4.66 (td, J=13.4, 4.5 Hz, 2H), 3.84 (s, 2H), 3.79 (s, 2H), 3.65 (t, J=6.9 Hz, 2H), 3.44-3.39 (m, 4H), 2.32 (t, J=6.6 Hz, 2H). MS m/z: 454 [M+H]⁺.

### Example 321: 6-[1-(2,2-difluoroethyl)-1H-pyrazolo[3,4-b]pyrazin-6-yl]-2-{[5-(trifluoromethyl)pyridin-2-yl]methyl}-2,6-diazaspiro[3.4]octane

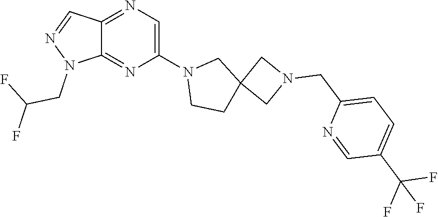 [SMILES: FC(F)Cn1ncc2ncc(N3CCC4(CN(Cc5ccc(C(F)(F)F)cn5)C4)C3)nc21]

Followed the General procedure A to afford the desired product as a colorless oil (10 mg, 36.47%). ¹H NMR (500 MHz, CDCl₃) δ 8.82-8.78 (m, 1H), 8.02 (s, 1H), 7.92 (d, J=5.2 Hz, 2H), 7.57 (d, J=8.2 Hz, 1H), 6.20 (tt, J=55.6, 4.5 Hz, 1H), 4.64 (td, J=13.3, 4.5 Hz, 2H), 4.00 (s, 2H), 3.80 (s, 2H), 3.63 (t, J=6.9 Hz, 2H), 3.60-3.53 (m, 4H), 2.32 (d, J=7.1 Hz, 2H). MS m/z: 454 [M+H]⁺.

### Example 322: 6-[1-(2,2-difluoroethyl)-1H-pyrazolo[3,4-b]pyrazin-6-yl]-2-[6-(trifluoromethyl)pyridine-3-carbonyl]-2,6-diazaspiro[3.4]octane

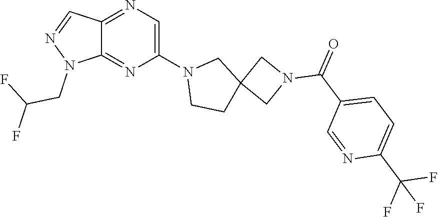 [SMILES: O=C(c1ccc(C(F)(F)F)nc1)N1CC2(CCN(c3cnc4cnn(CC(F)F)c4n3)C2)C1]

Followed the General procedure A to afford the desired product as a colorless oil (9 mg, 31.85%). ¹H NMR (500 MHz, CDCl₃) δ 8.97 (d, J=2.0 Hz, 1H), 8.21 (dd, J=8.1, 2.1 Hz, 1H), 8.06 (s, 1H), 7.96 (s, 1H), 7.79 (d, J=8.1 Hz, 1H), 6.22 (tt, J=55.6, 4.5 Hz, 1H), 4.66 (td, J=13.4, 4.5 Hz, 2H), 4.46-4.23 (m, 4H), 3.89 (s, 2H), 3.73 (dt, J=18.4, 6.8 Hz, 2H), 2.44-2.34 (m, 2H). MS m/z: 468 [M+H]⁺.

### Example 323: 6-[1-(2,2-difluoroethyl)-1H-pyrazolo[3,4-b]pyrazin-6-yl]-2-[5-(trifluoromethyl)pyridine-2-carbonyl]-2,6-diazaspiro[3.4]octane

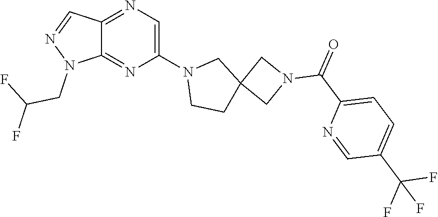 [SMILES: O=C(c1ccc(C(F)(F)F)cn1)N1CC2(CCN(c3cnc4cnn(CC(F)F)c4n3)C2)C1]

Followed the General procedure A to afford the desired product as a colorless oil (18 mg, 63.69%). ¹H NMR (500 MHz, CDCl₃) δ 8.83 (s, 1H), 8.30 (d, J=8.2 Hz, 1H), 8.08 (dd, J=8.2, 2.3 Hz, 1H), 8.06 (s, 1H), 7.97 (s, 1H), 6.22 (tt, J=55.6, 4.5 Hz, 1H), 4.83-4.72 (m, 2H), 4.67 (td, J=13.3, 4.5 Hz, 2H), 4.34-4.22 (m, 2H), 3.88 (q, J=11.2 Hz, 2H), 3.74 (dtd, J=17.8, 10.9, 6.8 Hz, 2H), 2.37 (t, J=6.8 Hz, 2H). MS m/z: 468 [M+H]⁺.

### Example 324: 8-[1-(2,2-difluoroethyl)-1H-pyrazolo[3,4-b]pyrazin-6-yl]-2-[2-(trifluoromethyl)pyrimidine-5-carbonyl]-2,8-diazaspiro[4.5]decane

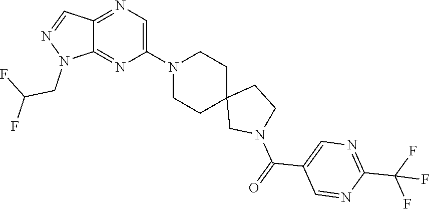 [SMILES: O=C(c1cnc(C(F)(F)F)nc1)N1CCC2(CCN(c3cnc4cnn(CC(F)F)c4n3)CC2)C1]

Followed the General procedure A to afford the desired product as a colorless oil (23 mg, 83.12%). ¹H NMR (500 MHz, CDCl₃) δ 9.09 (s, 2H), 8.26 (s, 1H), 8.06 (s, 1H), 6.22 (tt, J=55.6, 4.5 Hz, 1H), 4.67 (td, J=13.3, 4.5 Hz, 2H), 4.01-3.96 (m, 1H), 3.84 (t, J=7.3 Hz, 1H), 3.80-3.73 (m, 1H), 3.73-3.61 (m, 4H), 3.41 (s, 1H), 2.05-1.96 (m, 2H), 1.85-1.68 (m, 4H). MS m/z: 497 [M+H]⁺.

### Example 325: 6-[1-(2,2-difluoroethyl)-1H-pyrazolo[3,4-b]pyrazin-6-yl]-2-[2-(trifluoromethyl)pyrimidine-5-carbonyl]-2,6-diazaspiro[3.4]octane

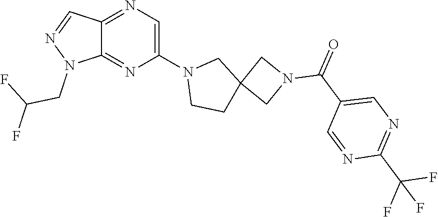 [SMILES: O=C(c1cnc(C(F)(F)F)nc1)N1CC2(CCN(c3cnc4cnn(CC(F)F)c4n3)C2)C1]

Followed the General procedure A to afford the desired product as a colorless oil (13 mg, 45.9%). ¹H NMR (500 MHz, CDCl₃) δ 9.17 (s, 2H), 8.07 (s, 1H), 7.96 (s, 1H), 6.22 (tt, J=55.6, 4.5 Hz, 1H), 4.67 (td, J=13.3, 4.5 Hz, 2H), 4.49-4.25 (m, 4H), 3.91 (s, 2H), 3.74 (dt, J=14.9, 6.8 Hz, 2H), 2.41 (q, J=6.9 Hz, 2H). MS m/z: 469 [M+H]⁺.

### Example 326: 7-[1-(2,2-difluoroethyl)-1H-pyrazolo[3,4-b]pyrazin-6-yl]-2-[2-(trifluoromethyl)pyrimidine-5-carbonyl]-2,7-diazaspiro[3.5]nonane

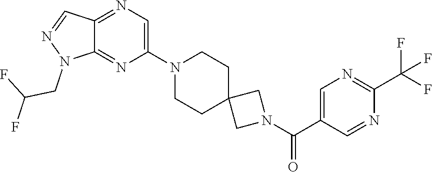 [SMILES: O=C(c1cnc(C(F)(F)F)nc1)N1CC2(CCN(c3cnc4cnn(CC(F)F)c4n3)CC2)C1]

Followed the General procedure A to afford the desired product as a colorless oil (12 mg, 42.88%). ¹H NMR (500 MHz, CDCl₃) δ 9.18 (s, 2H), 8.25 (s, 1H), 8.06 (s, 1H), 6.21 (tt, J=55.6, 4.5 Hz, 1H), 4.66 (td, J=13.3, 4.5 Hz, 2H), 4.17 (s, 2H), 4.09 (s, 2H), 3.85-3.66 (m, 4H), 2.04-1.93 (m, 4H). MS m/z: 483 [M+H]⁺.

### Example 327: 7-[1-(2,2-difluoroethyl)-1H-pyrazolo[3,4-b]pyrazin-6-yl]-2-[6-(trifluoromethyl)pyridine-3-carbonyl]-2,7-diazaspiro[3.5]nonane

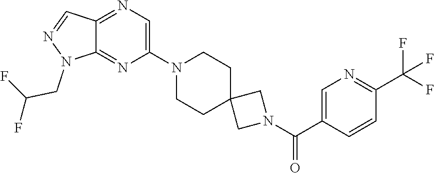 [SMILES: O=C(c1ccc(C(F)(F)F)nc1)N1CC2(CCN(c3cnc4cnn(CC(F)F)c4n3)CC2)C1]

Followed the General procedure A to afford the desired product as a white foam (11 mg, 39.39%). ¹H NMR (500 MHz, CDCl₃) δ 8.97 (d, J=2.1 Hz, 1H), 8.25 (s, 1H), 8.24-8.20 (m, 1H), 8.05 (s, 1H), 7.79 (dd, J=8.1, 0.9 Hz, 1H), 6.21 (tt, J=55.6, 4.5 Hz, 1H), 4.66 (td, J=13.3, 4.5 Hz, 2H), 4.13 (s, 2H), 4.07 (s, 2H), 3.83-3.66 (m, 4H), 2.02-1.91 (m, 4H). MS m/z: 482 [M+H]⁺.

### Example 328: 7-[1-(2,2-difluoroethyl)-1H-pyrazolo[3,4-b]pyrazin-6-yl]-2-[5-(trifluoromethyl)pyridine-2-carbonyl]-2,7-diazaspiro[3.5]nonane

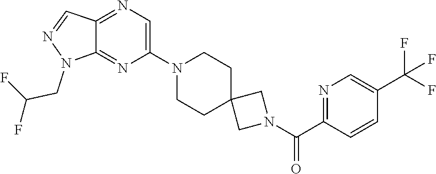

Followed the General procedure A to afford the desired product as a colorless oil (19 mg, 68.04%). ¹H NMR (500 MHz, CDCl₃) δ 8.86 (d, J=2.0 Hz, 1H), 8.29 (d, J=8.2 Hz, 1H), 8.26 (s, 1H), 8.08 (dd, J=8.3, 2.3 Hz, 1H), 8.05 (s, 1H), 6.21 (tt, J=55.6, 4.4 Hz, 1H), 4.66 (td, J=13.4, 4.5 Hz, 2H), 4.54 (s, 2H), 4.05 (s, 2H), 3.77 (t, J=5.5 Hz, 4H), 1.96 (dd, J=7.3, 4.1 Hz, 4H). MS m/z: 482 [M+H]⁺.

### Example 329: 2-[1-(2,2-difluoroethyl)-1H-pyrazolo[3,4-b]pyrazin-6-yl]-7-[2-(trifluoromethyl)pyrimidine-5-carbonyl]-2,7-diazaspiro[3.5]nonane

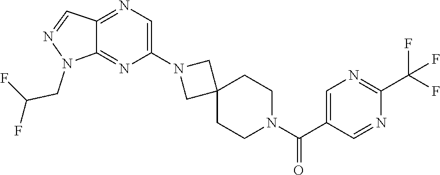

Followed the General procedure A to afford the desired product as a colorless oil (8 mg, 28.59%). ¹H NMR (500 MHz, CDCl₃) δ 8.97 (s, 2H), 8.07 (s, 1H), 7.81 (s, 1H), 6.22 (tt, J=55.6, 4.5 Hz, 1H), 4.66 (td, J=13.3, 4.5 Hz, 2H), 4.03 (d, J=11.1 Hz, 4H), 3.84 (s, 2H), 3.46 (s, 2H), 2.08-1.90 (m, 4H). MS m/z: 483 [M+H]⁺.

### Example 330: 2-[1-(2,2-difluoroethyl)-1H-pyrazolo[3,4-b]pyrazin-6-yl]-7-[5-(trifluoromethyl)pyridine-2-carbonyl]-2,7-diazaspiro[3.5]nonane

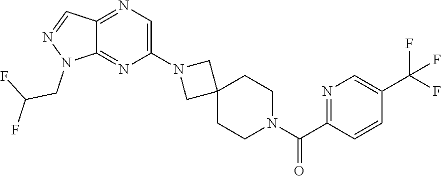 [SMILES: O=C(c1ccc(C(F)(F)F)cn1)N1CCC2(CC1)CN(c1cnc3cnn(CC(F)F)c3n1)C2]

Followed the General procedure A to afford the desired product as a colorless oil (9 mg, 32.23%). ¹H NMR (500 MHz, CDCl₃) δ 8.89-8.85 (m, 1H), 8.09-8.06 (m, 1H), 8.05 (s, 1H), 7.82-7.78 (m, 2H), 6.22 (tt, J=55.6, 4.5 Hz, 1H), 4.66 (td, J=13.3, 4.5 Hz, 2H), 4.07-3.96 (m, 4H), 3.83 (t, J=5.6 Hz, 2H), 3.58-3.51 (m, 2H), 2.05-1.92 (m, 4H). MS m/z: 482 [M+H]⁺.

### Example 331: 8-[1-(2,2-difluoroethyl)-1H-pyrazolo[3,4-b]pyrazin-6-yl]-2-[(5-fluoropyridin-3-yl)methyl]-2,8-diazaspiro[4.5]decan-1-one

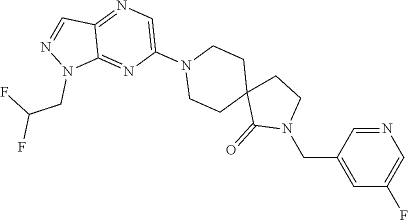 [SMILES: O=C1N(Cc2cncc(F)c2)CCC12CCN(c1cnc3cnn(CC(F)F)c3n1)CC2]

Followed the General procedure A to afford the desired product as a colorless oil (15 mg, 56.63%). ¹H NMR (500 MHz, CDCl₃) δ 8.45 (d, J=2.7 Hz, 1H), 8.36 (d, J=1.6 Hz, 1H), 8.25 (s, 1H), 8.04 (s, 1H), 7.39 (dt, J=6.8, 2.2 Hz, 1H), 6.22 (tt, J=55.6, 4.5 Hz, 1H), 4.66 (td, J=13.3, 4.5 Hz, 2H), 4.53 (s, 2H), 4.36 (dt, J=13.7, 4.7 Hz, 2H), 3.44 (ddd, J=13.6, 10.2, 3.3 Hz, 2H), 3.31 (t, J=6.9 Hz, 2H), 2.11-2.01 (m, 4H), 1.64-1.55 (m, 2H). MS m/z: 446 [M+H]⁺.

### Example 332: 8-[1-(2,2-difluoroethyl)-1H-pyrazolo[3,4-b]pyrazin-6-yl]-2-{[5-(trifluoromethyl)pyridin-3-yl]methyl}-2,8-diazaspiro[4.5]decan-1-one

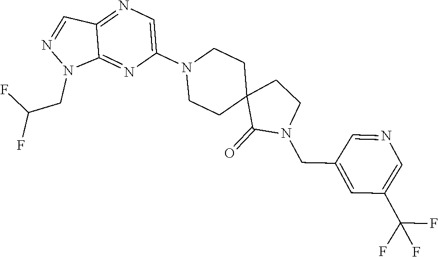 [SMILES: O=C1N(Cc2cncc(C(F)(F)F)c2)CCC12CCN(c1cnc3cnn(CC(F)F)c3n1)CC2]

Followed the General procedure A to afford the desired product as a colorless oil (14 mg, 47.52%). ¹H NMR (500 MHz, CDCl₃) δ 8.89-8.82 (m, 1H), 8.71 (d, J=2.1 Hz, 1H), 8.25 (s, 1H), 8.05 (s, 1H), 7.83 (d, J=2.3 Hz, 1H), 6.22 (tt, J=55.6, 4.5 Hz, 1H), 4.66 (td, J=13.3, 4.5 Hz, 2H), 4.58 (s, 2H), 4.36 (dt, J=13.6, 4.8 Hz, 2H), 3.45 (ddd, J=13.6, 10.1, 3.3 Hz, 2H), 3.31 (t, J=6.9 Hz, 2H), 2.12-2.01 (m, 4H), 1.65-1.55 (m, 2H). MS m/z: 496 [M+H]⁺.

### Example 333: 2-[(5-chloropyridin-3-yl)methyl]-8-[1-(2,2-difluoroethyl)-1H-pyrazolo[3,4-b]pyrazin-6-yl]-2,8-diazaspiro[4.5]decan-1-one

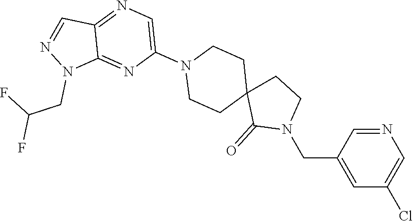 [SMILES: O=C1N(Cc2cncc(Cl)c2)CCC12CCN(c1cnc3cnn(CC(F)F)c3n1)CC2]

Followed the General procedure A to afford the desired product as a colorless oil (16 mg, 58.25%). ¹H NMR (500 MHz, CDCl₃) δ 8.54 (d, J=2.3 Hz, 1H), 8.40 (d, J=1.9 Hz, 1H), 8.25 (s, 1H), 8.04 (s, 1H), 7.62 (s, 1H), 6.21 (tt, J=55.6, 4.5 Hz, 1H), 4.66 (td, J=13.3, 4.5 Hz, 2H), 4.50 (s, 2H), 4.36 (dt, J=13.7, 4.7 Hz, 2H), 3.44 (ddd, J=13.6, 10.2, 3.3 Hz, 2H), 3.30 (t, J=6.9 Hz, 2H), 2.10-2.02 (m, 4H), 1.63-1.56 (m, 2H). MS m/z: 462 [M+H]⁺.

### Example 334: 8-[1-(2,2-difluoroethyl)-1H-pyrazolo[3,4-b]pyrazin-6-yl]-2-[(5-fluoropyridin-3-yl)methyl]-2,8-diazaspiro[4.5]decan-3-one

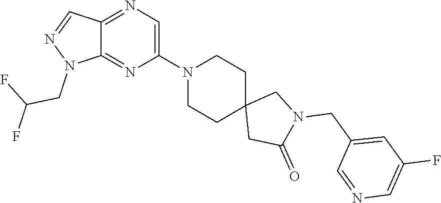 [SMILES: O=C1CC2(CCN(c3cnc4cnn(CC(F)F)c4n3)CC2)CN1Cc1cncc(F)c1]

Followed the General procedure A to afford the desired product as a colorless oil (12 mg, 45.3%). ¹H NMR (500 MHz, CDCl₃) δ 8.43 (d, J=2.7 Hz, 1H), 8.34 (d, J=1.7 Hz, 1H), 8.20 (s, 1H), 8.02 (s, 1H), 7.37 (ddd, J=8.8, 2.7, 1.8 Hz, 1H), 6.18 (tt, J=55.6, 4.5 Hz, 1H), 4.63 (td, J=13.4, 4.5 Hz, 2H), 4.51 (s, 2H), 3.87 (ddd, J=14.0, 6.5, 4.0 Hz, 2H), 3.57 (ddd, J=13.8, 8.5, 3.7 Hz, 2H), 3.15 (s, 2H), 2.46 (s, 2H), 1.74-1.63 (m, 4H). MS m/z: 446 [M+H]⁺.

### Example 335: 8-[1-(2,2-difluoroethyl)-1H-pyrazolo[3,4-b]pyrazin-6-yl]-2-{[5-(trifluoromethyl)pyridin-3-yl]methyl}-2,8-diazaspiro[4.5]decan-3-one

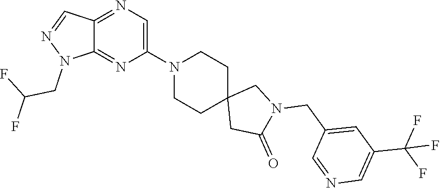 [SMILES: O=C1CC2(CCN(c3cnc4cnn(CC(F)F)c4n3)CC2)CN1Cc1cncc(C(F)(F)F)c1]

Followed the General procedure A to afford the desired product as a colorless oil (16 mg, 54.31%). ¹H NMR (500 MHz, CDCl₃) δ 8.90-8.82 (m, 1H), 8.73 (d, J=1.9 Hz, 1H), 8.22 (s, 1H), 8.04 (s, 1H), 7.86 (t, J=2.2 Hz, 1H), 6.20 (tt, J=55.6, 4.5 Hz, 1H), 4.65 (td, J=13.4, 4.5 Hz, 2H), 4.58 (s, 2H), 3.90 (ddd, J=13.8, 6.4, 4.0 Hz, 2H), 3.58 (ddd, J=13.8, 8.7, 3.6 Hz, 2H), 3.18 (s, 2H), 2.49 (s, 2H), 1.86-1.73 (m, 4H). MS m/z: 496 [M+H]⁺.

### Example 336: 2-[(5-chloropyridin-3-yl)methyl]-8-[1-(2,2-difluoroethyl)-1H-pyrazolo[3,4-b]pyrazin-6-yl]-2,8-diazaspiro[4.5]decan-3-one

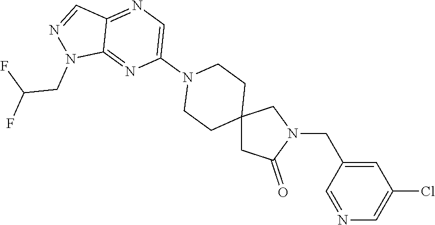 [SMILES: O=C1CC2(CCN(c3cnc4cnn(CC(F)F)c4n3)CC2)CN1Cc1cncc(Cl)c1]

Followed the General procedure A to afford the desired product as a colorless oil (16 mg, 58.25%). ¹H NMR (500 MHz, CDCl₃) δ 8.54 (d, J=2.3 Hz, 1H), 8.41 (d, J=1.9 Hz, 1H), 8.22 (s, 1H), 8.04 (s, 1H), 7.65 (t, J=2.1 Hz, 1H), 6.20 (tt, J=55.6, 4.5 Hz, 1H), 4.65 (td, J=13.3, 4.5 Hz, 2H), 4.50 (s, 2H), 3.89 (ddd, J=13.9, 6.4, 4.0 Hz, 2H), 3.59 (ddd, J=13.8, 8.5, 3.7 Hz, 2H), 3.17 (s, 2H), 2.48 (s, 2H), 1.77-1.65 (m, 4H). MS m/z: 462 [M+H]⁺.

### Example 337: 2-[1-(2,2-difluoroethyl)-1H-pyrazolo[3,4-b]pyrazin-6-yl]-6-[2-(trifluoromethyl)pyridin-4-yl]-2,6-diazaspiro[3.4]octan-5-one

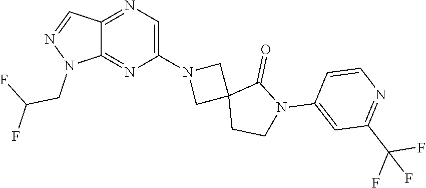 [SMILES: O=C1N(c2ccnc(C(F)(F)F)c2)CCC12CN(c1cnc3cnn(CC(F)F)c3n1)C2]

Followed the General procedure A to afford the desired product as a colorless oil (14 mg, 47.51%). ¹H NMR (500 MHz, CDCl₃) δ 8.70 (d, J=5.6 Hz, 1H), 8.09 (s, 1H), 8.05 (d, J=2.2 Hz, 1H), 7.87 (dd, J=5.7, 2.2 Hz, 1H), 7.85 (s, 1H), 6.21 (tt, J=55.6, 4.5 Hz, 1H), 4.67 (td, J=13.3, 4.5 Hz, 2H), 4.57 (d, J=8.4 Hz, 2H), 4.21 (d, J=8.4 Hz, 2H), 3.94 (t, J=6.9 Hz, 2H), 2.70 (t, J=6.9 Hz, 2H). MS m/z: 454 [M+H]⁺.

### Example 338: 2-[1-(2,2-difluoroethyl)-1H-pyrazolo[3,4-b]pyrazin-6-yl]-6-[2-(trifluoromethyl)pyridin-4-yl]-2,6-diazaspiro[3.4]octan-7-one

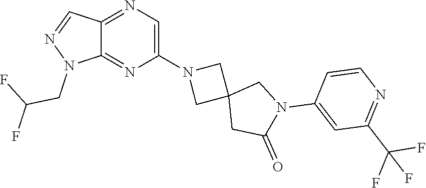 [SMILES: O=C1CC2(CN(c3cnc4cnn(CC(F)F)c4n3)C2)CN1c1ccnc(C(F)(F)F)c1]

Followed the General procedure A to afford the desired product as a colorless oil (10 mg, 33.93%). ¹H NMR (500 MHz, CDCl₃) δ 8.69 (d, J=5.5 Hz, 1H), 8.10 (s, 1H), 7.97 (d, J=2.0 Hz, 1H), 7.87-7.79 (m, 2H), 6.21 (tt, J=55.5, 4.4 Hz, 1H), 4.67 (td, J=13.3, 4.5 Hz, 2H), 4.33 (s, 4H), 4.21 (s, 2H), 3.07 (s, 2H). MS m/z: 454 [M+H]⁺.

### Example 339: 6-[1-(2,2-difluoroethyl)-1H-pyrazolo[3,4-b]pyrazin-6-yl]-2-[2-(trifluoromethyl)pyridin-4-yl]-2,6-diazaspiro[3.4]octan-5-one

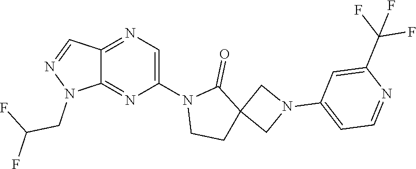 [SMILES: O=C1N(c2cnc3cnn(CC(F)F)c3n2)CCC12CN(c1ccnc(C(F)(F)F)c1)C2]

Followed the General procedure A to afford the desired product as a colorless oil (7 mg, 16.35%). ¹H NMR (500 MHz, CDCl₃) δ 9.78 (s, 1H), 8.31 (s, 1H), 8.30 (d, J=5.8 Hz, 1H), 6.93 (d, J=2.4 Hz, 1H), 6.69 (dd, J=5.9, 2.4 Hz, 1H), 6.22 (tt, J=55.3, 4.3 Hz, 1H), 4.78 (td, J=13.3, 4.4 Hz, 2H), 4.22 (ddd, J=8.0, 6.4, 1.7 Hz, 2H), 3.86 (q, J=11.4 Hz, 2H), 3.76 (dd, J=13.4, 8.1 Hz, 1H), 3.51 (dd, J=13.4, 3.9 Hz, 1H), 2.58 (ddd, J=13.7, 7.7, 6.1 Hz, 1H), 2.27-2.20 (m, 1H). MS m/z: 454 [M+H]⁺.

### Example 340: 6-[1-(2,2-difluoroethyl)-1H-pyrazolo[3,4-b]pyrazin-6-yl]-2-[2-(trifluoromethyl)pyridin-4-yl]-2,6-diazaspiro[3.4]octan-7-one

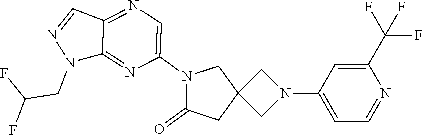 [SMILES: O=C1CC2(CN(c3ccnc(C(F)(F)F)c3)C2)CN1c1cnc2cnn(CC(F)F)c2n1]

Followed the General procedure A to afford the desired product as a colorless oil (3.3 mg, 13%). MS m/z: 454 [M+H]⁺.

### Example 341: 2-[1-(2,2-difluoroethyl)-1H-pyrazolo[3,4-b]pyrazin-6-yl]-6-[5-(trifluoromethyl)pyridin-2-yl]-2,6-diazaspiro[3.5]nonan-5-one

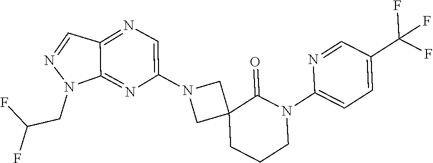 [SMILES: O=C1N(c2ccc(C(F)(F)F)cn2)CCCC12CN(c1cnc3cnn(CC(F)F)c3n1)C2]

Followed the General procedure A to afford the desired product as a light-yellow oil (23 mg, 79.3%). ¹H NMR (500 MHz, CDCl₃) δ 8.68 (dt, J=2.7, 0.9 Hz, 1H), 8.10-8.02 (m, 2H), 7.94-7.89 (m, 1H), 7.84 (s, 1H), 6.22 (tt, J=55.6, 4.5 Hz, 1H), 4.72-4.61 (m, 4H), 4.10-3.98 (m, 4H), 2.44-2.35 (m, 2H), 2.14-2.05 (m, 2H). MS m/z: 468 [M+H]⁺.

### Example 342: 6-[1-(2,2-difluoroethyl)-1H-pyrazolo[3,4-b]pyrazin-6-yl]-2-[6-(trifluoromethyl)pyridin-3-yl]-2,6-diazaspiro[3.5]nonan-5-one

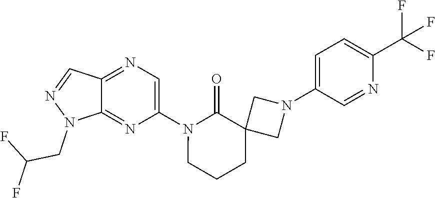 [SMILES: O=C1N(c2cnc3cnn(CC(F)F)c3n2)CCCC12CN(c1ccc(C(F)(F)F)nc1)C2]

Followed the General procedure A to afford the desired product as a light-yellow oil (19 mg, 89.2%). ¹H NMR (500 MHz, CDCl₃) δ 9.13 (s, 1H), 8.29 (s, 1H), 7.93 (d, J=2.8 Hz, 1H), 7.50 (d, J=8.5 Hz, 1H), 6.81 (dd, J=8.5, 2.8 Hz, 1H), 6.22 (tt, J=55.4, 4.4 Hz, 1H), 4.87-4.76 (m, 2H), 4.48 (d, J=7.0 Hz, 2H), 4.08 (t, J=6.1 Hz, 2H), 3.90 (d, J=7.0 Hz, 2H), 2.46-2.39 (m, 2H), 2.13 (td, J=7.3, 4.4 Hz, 2H). MS m/z: 468 [M+H]⁺.

### Example 343: 6-[1-(2,2-difluoroethyl)-1H-pyrazolo[3,4-b]pyrazin-6-yl]-2-[5-(trifluoromethyl)pyridin-2-yl]-2,6-diazaspiro[3.5]nonan-5-one

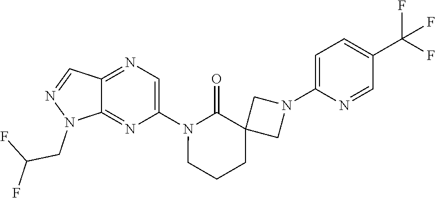 [SMILES: O=C1N(c2cnc3cnn(CC(F)F)c3n2)CCCC12CN(c1ccc(C(F)(F)F)cn1)C2]

Followed the General procedure A to afford the desired product as a light-yellow oil (18 mg, 84.51%). ¹H NMR (500 MHz, CDCl₃) δ 9.13 (s, 1H), 8.39 (dt, J=2.1, 1.0 Hz, 1H), 8.28 (s, 1H), 7.71-7.62 (m, 1H), 6.39 (d, J=8.8 Hz, 1H), 6.22 (tt, J=55.4, 4.4 Hz, 1H), 4.79 (td, J=13.2, 4.4 Hz, 2H), 4.61 (d, J=7.9 Hz, 2H), 4.07 (t, J=6.1 Hz, 2H), 3.98 (s, 2H), 2.46-2.37 (m, 2H), 2.18-2.05 (m, 2H). MS m/z: 468 [M+H]⁺.

### Example 344: 2-[1-(oxetan-3-yl)-1H-pyrazolo[3,4-b]pyrazin-6-yl]-6-[2-(trifluoromethyl)pyridin-4-yl]-2,6-diazaspiro[3.4]octane

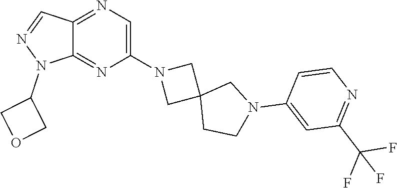 [SMILES: FC(F)(F)c1cc(N2CCC3(C2)CN(c2cnc4cnn(C5COC5)c4n2)C3)ccn1]

Followed the General procedure A to afford the desired product as a colorless oil (15 mg, 51.06%). ¹H NMR (500 MHz, CDCl₃) δ 8.33 (d, J=5.8 Hz, 1H), 8.13 (s, 1H), 7.81 (s, 1H), 6.77 (d, J=2.4 Hz, 1H), 6.52 (dd, J=6.0, 2.4 Hz, 1H), 5.96-5.90 (m, 1H), 5.31 (t, J=6.7 Hz, 2H), 5.08-5.01 (m, 2H), 4.27-4.17 (m, 4H), 3.68 (s, 2H), 3.54 (t, J=6.9 Hz, 2H), 2.41 (t, J=6.9 Hz, 2H). MS m/z: 432 [M+H]⁺.

### Example 345: 2-[3-methyl-1-(oxetan-3-yl)-1H-pyrazolo[3,4-b]pyrazin-6-yl]-6-[2-(trifluoromethyl)pyridin-4-yl]-2,6-diazaspiro[3.4]octane

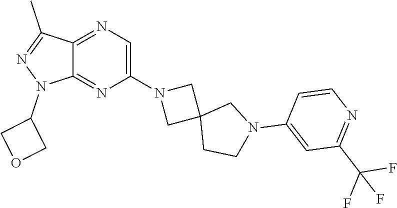 [SMILES: Cc1nn(C2COC2)c2nc(N3CC4(CCN(c5ccnc(C(F)(F)F)c5)C4)C3)cnc12]

Followed the General procedure A to afford the desired product as a colorless oil (8 mg, 26.38%). ¹H NMR (500 MHz, CDCl₃) δ 8.33 (d, J=4.9 Hz, 1H), 7.73 (s, 1H), 6.76 (s, 1H), 6.51 (d, J=5.2 Hz, 1H), 5.87 (tt, J=7.9, 6.7 Hz, 1H), 5.30 (t, J=6.6 Hz, 2H), 5.02 (dd, J=7.9, 6.5 Hz, 2H), 4.21 (q, J=8.6 Hz, 4H), 3.67 (s, 2H), 3.54 (t, J=6.7 Hz, 2H), 2.60 (s, 3H), 2.41 (t, J=6.5 Hz, 2H). MS m/z: 446 [M+H]⁺.

### Example 346: 2-[1-(oxetan-3-yl)-1H-pyrazolo[3,4-b]pyrazin-6-yl]-6-[2-(trifluoromethyl)pyridin-4-yl]-2,6-diazaspiro[3.4]octan-5-one

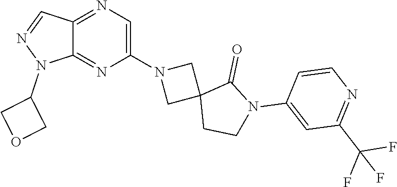 [SMILES: O=C1N(c2ccnc(C(F)(F)F)c2)CCC12CN(c1cnc3cnn(C4COC4)c3n1)C2]

Followed the General procedure A to afford the desired product as a colorless oil (13 mg, 44.9%). ¹H NMR (500 MHz, CDCl₃) δ 8.70 (d, J=5.6 Hz, 1H), 8.14 (s, 1H), 8.05 (d, J=2.2 Hz, 1H), 7.86 (dd, J=5.7, 2.2 Hz, 1H), 7.84 (s, 1H), 5.95 (tt, J=8.0, 6.7 Hz, 1H), 5.32 (t, J=6.7 Hz, 2H), 5.09-5.00 (m, 2H), 4.57 (d, J=8.4 Hz, 2H), 4.21 (d, J=8.7 Hz, 2H), 3.94 (t, J=6.9 Hz, 2H), 2.70 (t, J=6.9 Hz, 2H). MS m/z: 446 [M+H]⁺.

### Example 347: 2-[3-methyl-1-(oxetan-3-yl)-1H-pyrazolo[3,4-b]pyrazin-6-yl]-6-[2-(trifluoromethyl)pyridin-4-yl]-2,6-diazaspiro[3.4]octan-5-one

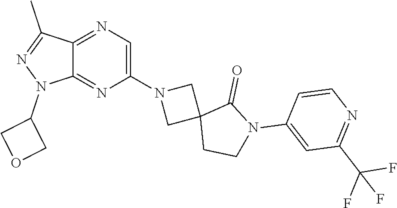 [SMILES: Cc1nn(C2COC2)c2nc(N3CC4(CCN(c5ccnc(C(F)(F)F)c5)C4=O)C3)cnc12]

Followed the General procedure A to afford the desired product as a colorless oil (12 mg, 40.18%). ¹H NMR (500 MHz, CDCl₃) δ 8.70 (d, J=5.6 Hz, 1H), 8.05 (d, J=2.2 Hz, 1H), 7.86 (dd, J=5.7, 2.2 Hz, 1H), 7.76 (s, 1H), 5.88 (tt, J=7.9, 6.7 Hz, 1H), 5.30 (t, J=6.7 Hz, 2H), 5.06-4.99 (m, 2H), 4.55 (d, J=8.3 Hz, 2H), 4.19 (d, J=8.4 Hz, 2H), 3.93 (t, J=6.9 Hz, 2H), 2.70 (t, J=6.9 Hz, 2H), 2.60 (s, 3H). MS m/z: 460 [M+H]⁺.

### Example 348: 2-[1-(oxetan-3-yl)-1H-pyrazolo[3,4-b]pyrazin-6-yl]-6-[2-(trifluoromethyl)pyridin-4-yl]-2,6-diazaspiro[3.4]octan-7-one

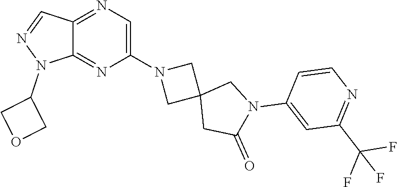 [SMILES: O=C1CC2(CN(c3cnc4cnn(C5COC5)c4n3)C2)CN1c1ccnc(C(F)(F)F)c1]

Followed the General procedure A to afford the desired product as a white solid (4 mg, 13.82%). ¹H NMR (500 MHz, CDCl₃) δ 8.69 (d, J=5.6 Hz, 1H), 8.15 (s, 1H), 7.96 (d, J=2.2 Hz, 1H), 7.83 (q, J=2.5 Hz, 2H), 5.94 (tt, J=8.0, 6.7 Hz, 1H), 5.31 (t, J=6.6 Hz, 2H), 5.07-5.01 (m, 2H), 4.31 (s, 4H), 4.20 (s, 2H), 3.06 (s, 2H). MS m/z: 446 [M+H]⁺.

### Example 349: 2-[3-methyl-1-(oxetan-3-yl)-1H-pyrazolo[3,4-b]pyrazin-6-yl]-6-[2-(trifluoromethyl)pyridin-4-yl]-2,6-diazaspiro[3.4]octan-7-one

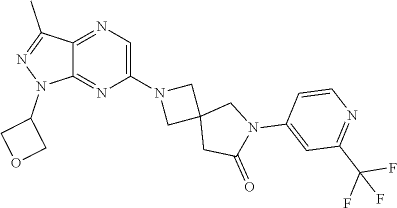 [SMILES: Cc1nn(C2COC2)c2nc(N3CC4(CC(=O)N(c5ccnc(C(F)(F)F)c5)C4)C3)cnc12]

Followed the General procedure A to afford the desired product as a colorless oil (8 mg, 26.79%). ¹H NMR (500 MHz, CDCl₃) δ 8.69 (d, J=5.7 Hz, 1H), 7.96 (d, J=2.2 Hz, 1H), 7.83 (dd, J=5.7, 2.1 Hz, 1H), 7.75 (s, 1H), 5.87 (tt, J=7.9, 6.7 Hz, 1H), 5.30 (t, J=6.6 Hz, 2H), 5.03 (dd, J=7.9, 6.6 Hz, 2H), 4.30 (s, 4H), 4.20 (s, 2H), 3.05 (s, 2H), 2.60 (s, 3H). MS m/z: 460 [M+H]⁺.

### Example 350: 2-[1-(2,2,2-trifluoroethyl)-1H-pyrazolo[3,4-b]pyrazin-6-yl]-6-[2-(trifluoromethyl)pyridin-4-yl]-2,6-diazaspiro[3.4]octane

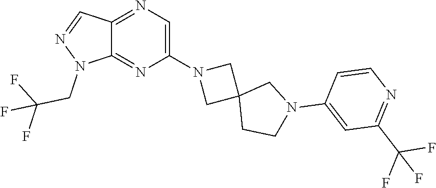 [SMILES: FC(F)(F)Cn1ncc2ncc(N3CC4(CCN(c5ccnc(C(F)(F)F)c5)C4)C3)nc21]

Followed the General procedure A to afford the desired product as a colorless oil (16 mg, 51.37%). ¹H NMR (500 MHz, CDCl₃) δ 8.33 (d, J=5.8 Hz, 1H), 8.11 (s, 1H), 7.83 (s, 1H), 6.76 (d, J=2.4 Hz, 1H), 6.50 (dd, J=5.9, 2.4 Hz, 1H), 4.89 (q, J=8.4 Hz, 2H), 4.24 (q, J=8.8 Hz, 4H), 3.68 (s, 2H), 3.53 (t, J=6.8 Hz, 2H), 2.41 (t, J=6.9 Hz, 2H). MS m/z: 458 [M+H]⁺.

### Example 351: 2-[1-(2,2-difluoroethyl)-1H-pyrazolo[3,4-d]pyrimidin-6-yl]-6-[2-(trifluoromethyl)pyridin-4-yl]-2,6-diazaspiro[3.4]octane

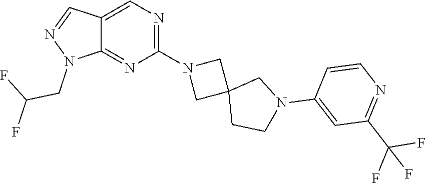 [SMILES: FC(F)Cn1ncc2cnc(N3CC4(CCN(c5ccnc(C(F)(F)F)c5)C4)C3)nc21]

Followed the General procedure A to afford the desired product as a colorless oil (14 mg, 46.79%). ¹H NMR (500 MHz, CDCl₃) δ 8.79 (s, 1H), 8.32 (d, J=5.8 Hz, 1H), 7.93 (s, 1H), 6.76 (d, J=2.4 Hz, 1H), 6.50 (dd, J=5.9, 2.4 Hz, 1H), 6.21 (tt, J=55.6, 4.4 Hz, 1H), 4.63 (td, J=13.3, 4.4 Hz, 2H), 4.27-4.14 (m, 4H), 3.64 (s, 2H), 3.51 (t, J=6.8 Hz, 2H), 2.38 (t, J=6.9 Hz, 2H). MS m/z: 440 [M+H]⁺.

### Example 352: 2-[1-(2,2-difluoroethyl)-3-methyl-1H-pyrazolo[3,4-d]pyrimidin-6-yl]-6-[2-(trifluoromethyl)pyridin-4-yl]-2,6-diazaspiro[3.4]octane

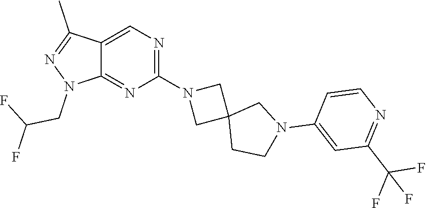 [SMILES: Cc1nn(CC(F)F)c2nc(N3CC4(CCN(c5ccnc(C(F)(F)F)c5)C4)C3)ncc12]

Followed the General procedure A to afford the desired product as a colorless oil (12 mg, 38.87%). ¹H NMR (500 MHz, CDCl₃) δ 8.68 (s, 1H), 8.31 (d, J=5.8 Hz, 1H), 6.76 (d, J=2.4 Hz, 1H), 6.49 (dd, J=5.9, 2.4 Hz, 1H), 6.18 (tt, J=55.6, 4.4 Hz, 1H), 4.54 (td, J=13.3, 4.4 Hz, 2H) 4.26-4.17 (m, 4H), 3.64 (s, 2H), 3.51 (t, J=6.9 Hz, 2H), 2.50 (s, 3H), 2.37 (t, J=6.9 Hz, 2H). MS m/z: 454 [M+H]⁺.

### Example 353: 2-[1-(2,2-difluoroethyl)-3-methyl-1H-pyrazolo[3,4-b]pyrazin-6-yl]-6-[2-(trifluoromethyl)pyridin-4-yl]-2,6-diazaspiro[3.4]octane

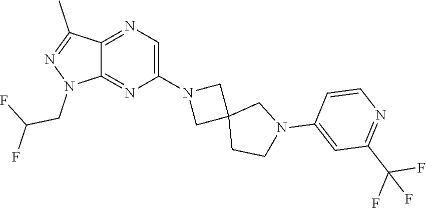 [SMILES: Cc1nn(CC(F)F)c2nc(N3CC4(CCN(c5ccnc(C(F)(F)F)c5)C4)C3)cnc12]

Followed the General procedure A to afford the desired product as a colorless oil (15 mg, 48.58%). ¹H NMR (500 MHz, CDCl₃) δ 8.33 (d, J=5.8 Hz, 1H), 7.75 (s, 1H), 6.76 (d, J=2.5 Hz, 1H), 6.50 (dd, J=5.8, 2.4 Hz, 1H), 6.19 (tt, J=55.8, 4.6 Hz, 1H), 4.59 (td, J=13.4, 4.5 Hz, 2H), 4.27-4.16 (m, 4H), 3.67 (s, 2H), 3.53 (t, J=6.9 Hz, 2H), 2.55 (s, 3H), 2.41 (t, J=6.9 Hz, 2H). MS m/z: 454 [M+H]⁺.

### Example 354: 8-[1-(2,2-difluoroethyl)-1H-pyrazolo[3,4-b]pyrazin-6-yl]-2-{[6-(trifluoromethyl)pyridin-2-yl]methyl}-2,8-diazaspiro[4.5]decan-1-one

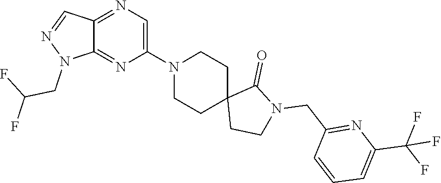 [SMILES: O=C1N(Cc2cccc(C(F)(F)F)n2)CCC12CCN(c1cnc3cnn(CC(F)F)c3n1)CC2]

Followed the General procedure A to afford the desired product as a colorless oil (13 mg, 44.13%). ¹H NMR (500 MHz, CDCl₃) δ 8.24 (s, 1H), 8.04 (s, 1H), 7.91-7.81 (m, 1H), 7.60 (dd, J=7.8, 0.9 Hz, 1H), 7.43 (d, J=7.9 Hz, 1H), 6.22 (tt, J=55.6, 4.5 Hz, 1H), 4.72-4.61 (m, 4H), 4.39-4.23 (m, 2H), 3.54-3.44 (m, 4H), 2.13-2.00 (m, 4H), 1.65 (dd, J=5.6, 3.5 Hz, 2H). MS m/z: 496 [M+H]⁺.

### Example 355: 8-[1-(2,2-difluoroethyl)-1H-pyrazolo[3,4-b]pyrazin-6-yl]-2-{[4-(trifluoromethyl)pyridin-2-yl]methyl}-2,8-diazaspiro[4.5]decan-1-one

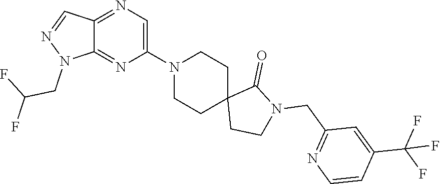 [SMILES: O=C1N(Cc2cc(C(F)(F)F)ccn2)CCC12CCN(c1cnc3cnn(CC(F)F)c3n1)CC2]

Followed the General procedure A to afford the desired product as a colorless oil (20 mg, 67.89%). ¹H NMR (500 MHz, CDCl₃) δ 8.75 (d, J=5.0 Hz, 1H), 8.25 (s, 1H), 8.04 (s, 1H), 7.51-7.40 (m, 2H), 6.22 (tt, J=55.6, 4.5 Hz, 1H), 4.69 (s, 2H), 4.68-4.62 (m, 2H), 4.35 (td, J=13.6, 4.8 Hz, 2H), 3.51-3.41 (m, 4H), 2.13-1.99 (m, 6H). MS m/z: 496 [M+H]⁺.

### Example 356: 8-[1-(2,2-difluoroethyl)-1H-pyrazolo[3,4-b]pyrazin-6-yl]-2-[(6-ethoxypyridin-3-yl)methyl]-2,8-diazaspiro[4.5]decan-1-one

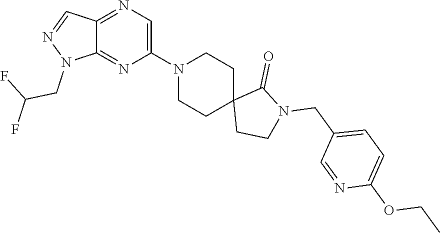 [SMILES: CCOc1ccc(CN2CCC3(CCN(c4cnc5cnn(CC(F)F)c5n4)CC3)C2=O)cn1]

Followed the General procedure A to afford the desired product as a colorless oil (17 mg, 60.63%). ¹H NMR (500 MHz, CDCl₃) δ 8.24 (s, 1H), 8.06-7.99 (m, 2H), 7.49 (dd, J=8.6, 2.4 Hz, 1H), 6.72 (d, J=8.5 Hz, 1H), 6.21 (tt, J=55.6, 4.5 Hz, 1H), 4.65 (td, J=13.3, 4.5 Hz, 2H), 4.40 (s, 2H), 4.38-4.31 (m, 4H), 3.42 (ddd, J=13.6, 10.2, 3.3 Hz, 2H), 3.24 (t, J=6.9 Hz, 2H), 2.07-1.98 (m, 4H), 1.60-1.52 (m, 2H), 1.40 (t, J=7.1 Hz, 3H). MS m/z: 472 [M+H]⁺.

### Example 357: 8-[1-(2,2-difluoroethyl)-1H-pyrazolo[3,4-b]pyrazin-6-yl]-2-{[2-(trifluoromethyl)pyridin-4-yl]methyl}-2,8-diazaspiro[4.5]decan-1-one

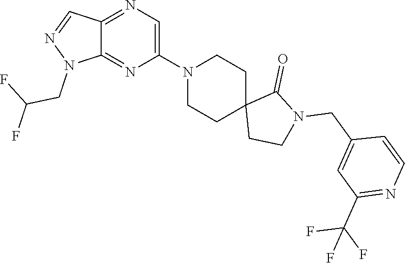 [SMILES: O=C1N(Cc2ccnc(C(F)(F)F)c2)CCC12CCN(c1cnc3cnn(CC(F)F)c3n1)CC2]

Followed the General procedure A to afford the desired product as a colorless oil (12 mg, 40.73%). ¹H NMR (500 MHz, CDCl₃) δ 8.72 (d, J=5.0 Hz, 1H), 8.25 (s, 1H), 8.05 (s, 1H), 7.58-7.49 (m, 1H), 7.39-7.32 (m, 1H), 6.22 (tt, J=55.6, 4.5 Hz, 1H), 4.66 (td, J=13.3, 4.5 Hz, 2H), 4.57 (s, 2H), 4.37 (dt, J=13.7, 4.7 Hz, 2H), 3.46 (ddd, J=13.5, 10.2, 3.3 Hz, 2H), 3.32 (t, J=6.9 Hz, 2H), 2.17-2.02 (m, 4H). MS m/z: 496 [M+H]⁺.

### Example 358: 8-[1-(2,2-difluoroethyl)-1H-pyrazolo[3,4-b]pyrazin-6-yl]-2-[(3,5-difluorophenyl)methyl]-2,8-diazaspiro[4.5]decan-1-one

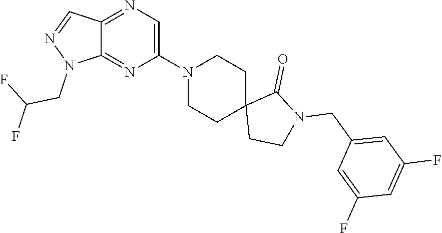 [SMILES: O=C1N(Cc2cc(F)cc(F)c2)CCC12CCN(c1cnc3cnn(CC(F)F)c3n1)CC2]

Followed the General procedure A to afford the desired product as a colorless oil (21 mg, 76.37%). ¹H NMR (500 MHz, CDCl₃) δ 8.75 (d, J=5.0 Hz, 1H), 8.25 (s, 1H), 8.04 (s, 1H), 7.51-7.40 (m, 2H), 6.22 (tt, J=55.6, 4.5 Hz, 1H), 4.69 (s, 2H), 4.68-4.62 (m, 2H), 4.35 (td, J=13.6, 4.8 Hz, 2H), 3.51-3.41 (m, 4H), 2.13-1.99 (m, 6H). MS m/z: 463 [M+H]⁺.

### Example 359: 8-[1-(2,2-difluoroethyl)-1H-pyrazolo[3,4-b]pyrazin-6-yl]-2-{[4-(trifluoromethyl)pyridin-2-yl]methyl}-2,8-diazaspiro[4.5]decan-3-one

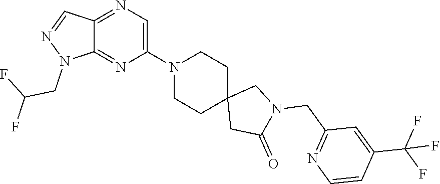 [SMILES: O=C1CC2(CCN(c3cnc4cnn(CC(F)F)c4n3)CC2)CN1Cc1cc(C(F)(F)F)ccn1]

Followed the General procedure A to afford the desired product as a colorless oil (22 mg, 74.67%). ¹H NMR (500 MHz, CDCl₃) δ 8.75 (d, J=5.1 Hz, 1H), 8.23 (s, 1H), 8.04 (s, 1H), 7.50 (d, J=1.7 Hz, 1H), 7.47 (dd, J=4.9, 1.5 Hz, 1H), 6.20 (tt, J=55.6, 4.4 Hz, 1H), 4.70 (s, 2H), 4.65 (td, J=13.4, 4.5 Hz, 2H), 3.86 (ddd, J=13.7, 6.6, 4.1 Hz, 2H), 3.65 (ddd, J=13.8, 8.1, 3.9 Hz, 2H), 3.34 (s, 2H), 2.49 (s, 2H), 1.81-1.74 (m, 4H). MS m/z: 496 [M+H]⁺.

### Example 360: 8-[1-(2,2-difluoroethyl)-1H-pyrazolo[3,4-b]pyrazin-6-yl]-2-{[6-(trifluoromethyl)pyridin-2-yl]methyl}-2,8-diazaspiro[4.5]decan-3-one

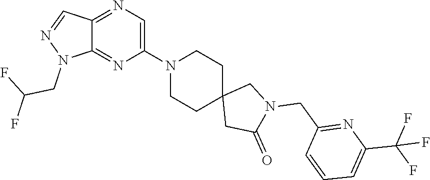 [SMILES: O=C1CC2(CCN(c3cnc4cnn(CC(F)F)c4n3)CC2)CN1Cc1cccc(C(F)(F)F)n1]

Followed the General procedure A to afford the desired product as a colorless oil (20 mg, 67.89%). ¹H NMR (500 MHz, CDCl₃) δ 8.23 (s, 1H), 8.04 (s, 1H), 7.87 (t, J=7.8 Hz, 1H), 7.61 (d, J=7.7 Hz, 1H), 7.48 (d, J=7.8 Hz, 1H), 6.21 (tt, J=55.6, 4.5 Hz, 1H), 4.67 (s, 2H), 4.65 (td, td, J=13.3, 4.4 Hz, 2H), 3.76 (ddt, J=36.0, 13.9, 5.7 Hz, 4H), 3.45 (s, 2H), 2.45 (s, 2H), 1.79 (t, J=5.7 Hz, 4H). MS m/z: 496 [M+H]⁺.

### Example 361: 8-[1-(2,2-difluoroethyl)-1H-pyrazolo[3,4-b]pyrazin-6-yl]-2-[(6-ethoxypyridin-3-yl)methyl]-2,8-diazaspiro[4.5]decan-3-one

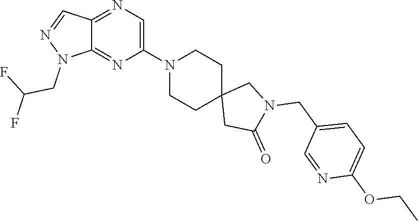 [SMILES: CCOc1ccc(CN2CC3(CCN(c4cnc5cnn(CC(F)F)c5n4)CC3)CC2=O)cn1]

Followed the General procedure A to afford the desired product as a colorless oil (17 mg, 60.63%). ¹H NMR (500 MHz, CDCl₃) δ 8.21 (s, 1H), 8.07-7.98 (m, 2H), 7.53 (dd, J=8.5, 2.5 Hz, 1H), 6.77-6.70 (m, 1H), 6.19 (tt, J=55.6, 4.5 Hz, 1H), 4.64 (td, J=13.3, 4.5 Hz, 2H), 4.40 (s, 2H), 4.35 (q, J=7.0 Hz, 2H), 3.84 (ddd, J=13.8, 6.5, 4.0 Hz, 2H), 3.59 (ddd, J=13.7, 8.4, 3.7 Hz, 2H), 3.12 (s, 2H), 2.44 (s, 2H), 1.77-1.68 (m, 4H), 1.40 (t, J=7.1 Hz, 3H). MS m/z: 472 [M+H]⁺.

### Example 362: 8-[1-(2,2-difluoroethyl)-1H-pyrazolo[3,4-b]pyrazin-6-yl]-2-{[2-(trifluoromethyl)pyridin-4-yl]methyl}-2,8-diazaspiro[4.5]decan-3-one

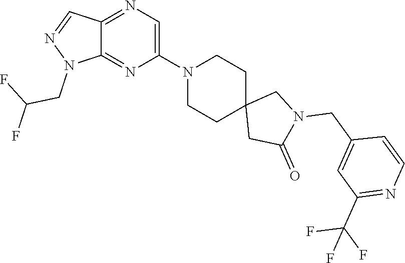 [SMILES: O=C1CC2(CCN(c3cnc4cnn(CC(F)F)c4n3)CC2)CN1Cc1ccnc(C(F)(F)F)c1]

Followed the General procedure A to afford the desired product as a colorless oil (12 mg, 40.73%). ¹H NMR (500 MHz, CDCl₃) δ 8.73 (d, J=5.0 Hz, 1H), 8.23 (s, 1H), 8.04 (s, 1H), 7.55 (d, J=1.6 Hz, 1H), 7.39 (dd, J=4.9, 1.6 Hz, 1H), 6.20 (tt, J=55.6, 4.5 Hz, 1H), 4.65 (td, J=13.4, 4.5 Hz, 2H), 4.57 (s, 2H), 3.91 (ddd, J=13.9, 6.4, 4.0 Hz, 2H), 3.60 (ddd, J=13.9, 8.7, 3.6 Hz, 2H), 3.19 (s, 2H), 2.52 (s, 2H), 1.82-1.71 (m, 4H). MS m/z: 496 [M+H]⁺.

### Example 363: 8-[1-(2,2-difluoroethyl)-1H-pyrazolo[3,4-b]pyrazin-6-yl]-2-[(3,5-difluorophenyl)methyl]-2,8-diazaspiro[4.5]decan-3-one

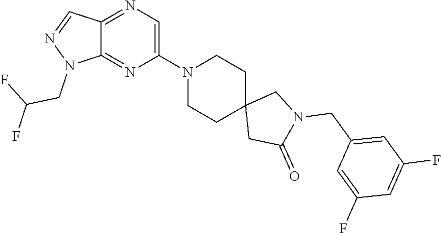 [SMILES: O=C1CC2(CCN(c3cnc4cnn(CC(F)F)c4n3)CC2)CN1Cc1cc(F)cc(F)c1]

Followed the General procedure A to afford the desired product as a colorless oil (23 mg, 83.64%). ¹H NMR (500 MHz, CDCl₃) δ 8.22 (s, 1H), 8.04 (s, 1H), 6.81-6.72 (m, 3H), 6.20 (tt, J=55.6, 4.5 Hz, 1H), 4.65 (td, J=13.4, 4.5 Hz, 2H), 4.45 (s, 2H), 3.88 (ddd, J=13.8, 6.5, 4.0 Hz, 2H), 3.60 (ddd, J=13.9, 8.6, 3.7 Hz, 2H), 3.15 (s, 2H), 2.48 (s, 2H), 1.79-1.68 (m, 4H). MS m/z: 463 [M+H]⁺.

### Example 364: 8-[1-(2,2-difluoroethyl)-1H-pyrazolo[3,4-b]pyrazin-6-yl]-2-[(4-ethyl-1,3-thiazol-2-yl)methyl]-2,8-diazaspiro[4.5]decan-1-one

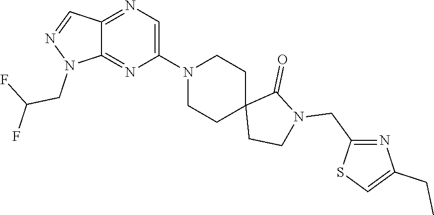 [SMILES: CCc1csc(CN2CCC3(CCN(c4cnc5cnn(CC(F)F)c5n4)CC3)C2=O)n1]

Followed the General procedure A to afford the desired product as a colorless oil (16 mg, 58.3%). ¹H NMR (500 MHz, CDCl₃) δ 8.24 (s, 1H), 8.04 (s, 1H), 6.86 (t, J=1.0 Hz, 1H), 6.21 (tt, J=55.6, 4.5 Hz, 1H), 4.77 (s, 2H), 4.66 (td, J=13.3, 4.5 Hz, 2H), 4.33 (dt, J=13.6, 4.9 Hz, 2H), 3.55-3.40 (m, 4H), 2.79 (qd, J=7.6, 1.0 Hz, 2H), 2.05 (ddd, J=12.9, 8.4, 3.7 Hz, 4H), 1.62 (s, 2H), 1.29 (t, J=7.5 Hz, 3H). MS m/z: 462 [M+H]⁺.

### Example 365: 8-[1-(2,2-difluoroethyl)-1H-pyrazolo[3,4-b]pyrazin-6-yl]-2-{[4-(trifluoromethyl)-1,3-thiazol-2-yl]methyl}-2,8-diazaspiro[4.5]decan-1-one

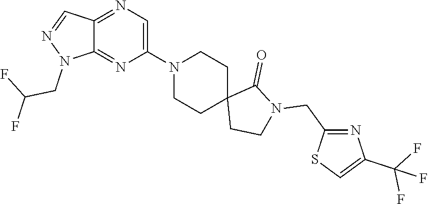 [SMILES: O=C1N(Cc2nc(C(F)(F)F)cs2)CCC12CCN(c1cnc3cnn(CC(F)F)c3n1)CC2]

Followed the General procedure A to afford the desired product as a colorless oil (6 mg, 20%). ¹H NMR (500 MHz, CDCl₃) δ 8.24 (s, 1H), 8.04 (s, 1H), 7.76 (d, J=1.0 Hz, 1H), 6.21 (tt, J=55.6, 4.5 Hz, 1H), 4.82 (s, 2H), 4.66 (td, J=13.3, 4.5 Hz, 2H), 4.33 (dt, J=13.7, 4.8 Hz, 2H), 3.55-3.43 (m, 4H), 2.10 (t, J=6.9 Hz, 2H), 2.05 (ddd, J=13.8, 9.9, 4.0 Hz, 2H), 1.64 (d, J=5.3 Hz, 2H). MS m/z: 502 [M+H]⁺.

### Example 366: 8-[1-(2,2-difluoroethyl)-1H-pyrazolo[3,4-b]pyrazin-6-yl]-2-{[2-(trifluoromethyl)-1,3-thiazol-5-yl]methyl}-2,8-diazaspiro[4.5]decan-1-one

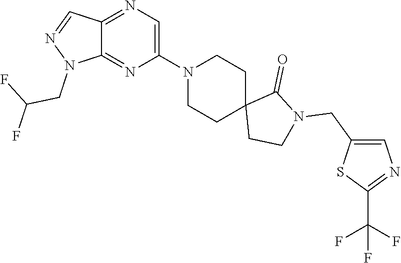 [SMILES: O=C1N(Cc2cnc(C(F)(F)F)s2)CCC12CCN(c1cnc3cnn(CC(F)F)c3n1)CC2]

Followed the General procedure A to afford the desired product as a colorless oil (4 mg, 13%). MS m/z: 502 [M+H]⁺.

### Example 367: 8-[1-(2,2-difluoroethyl)-1H-pyrazolo[3,4-b]pyrazin-6-yl]-2-[(4-ethyl-1,3-thiazol-2-yl)methyl]-2,8-diazaspiro[4.5]decan-3-one

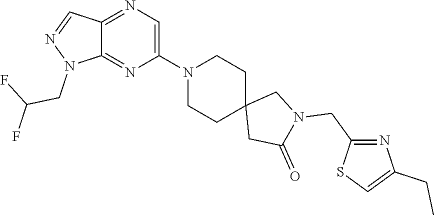 [SMILES: CCc1csc(CN2CC3(CCN(c4cnc5cnn(CC(F)F)c5n4)CC3)CC2=O)n1]

Followed the General procedure A to afford the desired product as a colorless oil (19 mg, 69.23%). ¹H NMR (500 MHz, CDCl₃) δ 8.22 (s, 1H), 8.04 (s, 1H), 6.87 (s, 1H), 6.20 (tt, J=55.5, 4.4 Hz, 1H), 4.78 (s, 2H), 4.65 (td, J=13.3, 4.5 Hz, 2H), 3.85-3.80 (m, 2H), 3.70-3.65 (m, 2H), 3.35 (s, 2H), 2.79 (qd, J=7.6, 1.0 Hz, 2H), 2.45 (s, 2H), 1.77 (q, J=5.6 Hz, 4H), 1.29 (s, 3H). MS m/z: 462 [M+H]⁺.

### Example 368: 8-[1-(2,2-difluoroethyl)-1H-pyrazolo[3,4-b]pyrazin-6-yl]-2-{[4-(trifluoromethyl)-1,3-thiazol-2-yl]methyl}-2,8-diazaspiro[4.5]decan-3-one

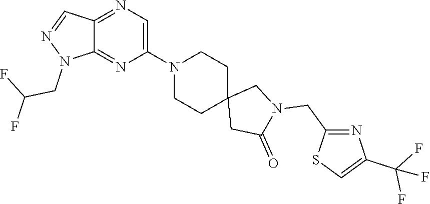 [SMILES: O=C1CC2(CCN(c3cnc4cnn(CC(F)F)c4n3)CC2)CN1Cc1nc(C(F)(F)F)cs1]

Followed the General procedure A to afford the desired product as a colorless oil (8 mg, 26.83%). ¹H NMR (500 MHz, CDCl₃) δ 8.23 (s, 1H), 8.05 (s, 1H), 7.77 (q, J=0.9 Hz, 1H), 6.20 (tt, J=55.6, 4.5 Hz, 1H), 4.82 (s, 2H), 4.65 (td, J=13.3, 4.5 Hz, 2H), 3.89-3.78 (m, 2H), 3.69 (dt, J=13.9, 5.4 Hz, 2H), 3.41 (s, 2H), 2.46 (s, 2H), 1.78 (t, J=5.5 Hz, 4H). MS m/z: 502 [M+H]⁺.

### Example 369: 8-[1-(2,2-difluoroethyl)-1H-pyrazolo[3,4-b]pyrazin-6-yl]-2-{[2-(trifluoromethyl)-1,3-thiazol-5-yl]methyl}-2,8-diazaspiro[4.5]decan-3-one

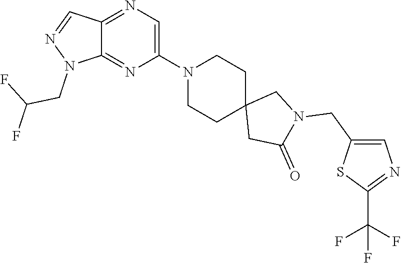 [SMILES: O=C1CC2(CCN(c3cnc4cnn(CC(F)F)c4n3)CC2)CN1Cc1cnc(C(F)(F)F)s1]

Followed the General procedure A to afford the desired product as a colorless oil (3 mg, 10%). MS m/z: 502 [M+H]⁺.

### Example 370: 6-[1-(2,2-difluoroethyl)-1H-pyrazolo[3,4-b]pyrazin-6-yl]-2-[4-(trifluoromethoxy)benzoyl]-2,6-diazaspiro[3.4]octane

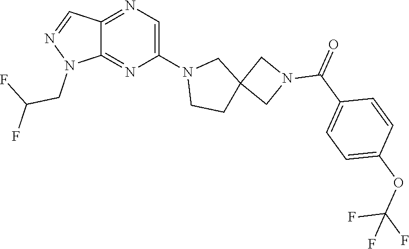 [SMILES: O=C(c1ccc(OC(F)(F)F)cc1)N1CC2(CCN(c3cnc4cnn(CC(F)F)c4n3)C2)C1]

Followed the General procedure A to afford the desired product as a off-white wax (22 mg, 78.16%). ¹H NMR (500 MHz, CDCl₃) δ 8.06 (s, 1H), 7.95 (s, 1H), 7.72 (d, J=2.1 Hz, 1H), 7.71 (d, J=2.1 Hz, 1H), 7.29-7.27 (m, 1H), 7.26 (s, 1H), 6.22 (tt, J=55.6, 4.5 Hz, 1H), 4.66 (td, J=13.4, 4.5 Hz, 2H), 4.40-4.19 (m, 4H), 3.87 (d, J=2.3 Hz, 2H), 3.71 (d, J=22.7 Hz, 2H), 2.35 (q, J=6.8 Hz, 2H). MS m/z: 483 [M+H]⁺.

### Example 371: 2-(3-chlorobenzoyl)-6-[1-(2,2-difluoroethyl)-1H-pyrazolo[3,4-b]pyrazin-6-yl]-2,6-diazaspiro[3.4]octane

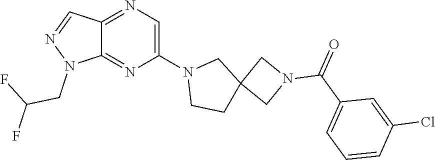 [SMILES: O=C(c1cccc(Cl)c1)N1CC2(CCN(c3cnc4cnn(CC(F)F)c4n3)C2)C1]

Followed the General procedure A to afford the desired product as a off-white wax (16 mg, 63.04%). ¹H NMR (500 MHz, CDCl₃) δ 8.05 (s, 1H), 7.95 (s, 1H), 7.64 (t, J=1.9 Hz, 1H), 7.54 (dt, J=7.7, 1.4 Hz, 1H), 7.45 (ddd, J=8.0, 2.1, 1.1 Hz, 1H), 7.37 (t, J=7.8 Hz, 1H), 6.22 (tt, J=55.6, 4.5 Hz, 1H), 4.66 (td, J=13.4, 4.5 Hz, 2H), 4.37-4.21 (m, 4H), 3.86 (d, J=4.4 Hz, 2H), 3.71 (dt, J=22.2, 7.1 Hz, 2H), 2.35 (q, J=6.6 Hz, 2H). MS m/z: 433 [M+H]⁺.

### Example 372: 6-[1-(2,2-difluoroethyl)-1H-pyrazolo[3,4-b]pyrazin-6-yl]-2-[4-(trifluoromethyl)benzenesulfonyl]-2,6-diazaspiro[3.4]octane

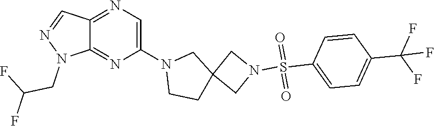 [SMILES: O=S(=O)(c1ccc(C(F)(F)F)cc1)N1CC2(CCN(c3cnc4cnn(CC(F)F)c4n3)C2)C1]

Followed the General procedure A to afford the desired product as a white solid (13.3 mg, 43.78%). ¹H NMR (500 MHz, CDCl₃) δ 8.04 (s, 1H), 8.03-7.97 (m, 2H), 7.89 (t, J=4.1 Hz, 3H), 6.20 (tt, J=55.6, 4.5 Hz, 1H), 4.63 (td, J=13.3, 4.5 Hz, 2H), 3.91-3.82 (m, 4H), 3.69-3.59 (m, 4H), 2.19 (t, J=6.9 Hz, 2H). MS m/z: 503 [M+H]⁺.

### Example 373: 6-[1-(2,2-difluoroethyl)-1H-pyrazolo[3,4-b]pyrazin-6-yl]-2-(4-fluorobenzenesulfonyl)-2,6-diazaspiro[3.4]octane

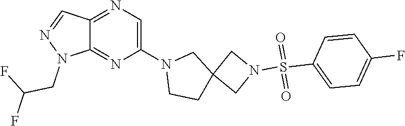 [SMILES: O=S(=O)(c1ccc(F)cc1)N1CC2(CCN(c3cnc4cnn(CC(F)F)c4n3)C2)C1]

Followed the General procedure A to afford the desired product as a white solid (14 mg, 51.17%). ¹H NMR (500 MHz, CDCl₃) δ 8.04 (s, 1H), 7.93-7.89 (m, 2H), 7.88 (s, 1H), 7.33-7.28 (m, 2H), 6.20 (tt, J=55.6, 4.5 Hz, 1H), 4.64 (td, J=13.4, 4.5 Hz, 2H), 3.81 (s, 4H), 3.66-3.57 (m, 4H), 2.17 (t, J=6.9 Hz, 2H). MS m/z: 453 [M+H]⁺.

### Example 374: 6-[1-(2,2-difluoroethyl)-1H-pyrazolo[3,4-b]pyrazin-6-yl]-2-[3-(trifluoromethyl)benzenesulfonyl]-2,6-diazaspiro[3.4]octane

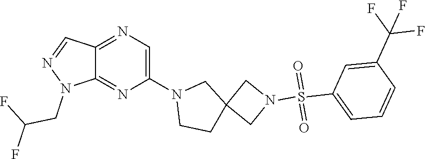 [SMILES: O=S(=O)(c1cccc(C(F)(F)F)c1)N1CC2(CCN(c3cnc4cnn(CC(F)F)c4n3)C2)C1]

Followed the General procedure A to afford the desired product as a off-white oil (13.7 mg, 45.09%). ¹H NMR (500 MHz, CDCl₃) δ 8.15 (td, J=1.8, 1.0 Hz, 1H), 8.08 (dt, J=7.8, 1.6 Hz, 1H), 8.04 (s, 1H), 7.96 (ddt, J=7.8, 1.8, 1.0 Hz, 1H), 7.88 (s, 1H), 7.81-7.75 (m, 1H), 6.20 (tt, J=55.6, 4.5 Hz, 1H), 4.64 (td, J=13.3, 4.5 Hz, 2H), 3.91-3.83 (m, 4H), 3.67-3.60 (m, 4H), 2.19 (t, J=7.0 Hz, 2H). MS m/z: 503 [M+H]⁺.

### Example 375: 6-[1-(2,2-difluoroethyl)-1H-pyrazolo[3,4-b]pyrazin-6-yl]-2-(3-fluorobenzenesulfonyl)-2,6-diazaspiro[3.4]octane

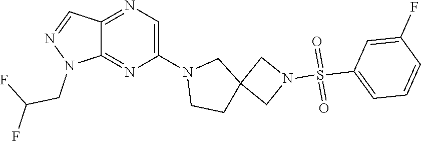 [SMILES: O=S(=O)(c1cccc(F)c1)N1CC2(CCN(c3cnc4cnn(CC(F)F)c4n3)C2)C1]

Followed the General procedure A to afford the desired product as a white solid (13.5 mg, 49.35%). ¹H NMR (500 MHz, CDCl₃) δ 8.04 (s, 1H), 7.88 (s, 1H), 7.68 (dt, J=7.8, 1.3 Hz, 1H), 7.65-7.61 (m, 1H), 7.59 (ddd, J=7.9, 2.6, 1.5 Hz, 1H), 7.42 (tdd, J=8.2, 2.6, 1.1 Hz, 1H), 6.20 (tt, J=55.6, 4.5 Hz, 1H), 4.64 (td, J=13.4, 4.5 Hz, 2H), 3.84 (s, 4H), 3.66-3.56 (m, 4H), 2.17 (t, J=6.9 Hz, 2H). MS m/z: 453 [M+H]⁺.

### Example 376: 2-[1-(oxetan-3-yl)-1H-pyrazolo[3,4-d]pyrimidin-6-yl]-6-[2-(trifluoromethyl)pyridin-4-yl]-2,6-diazaspiro[3.4]octane

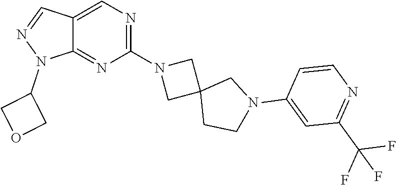

Followed the General procedure A to afford the desired product as a colorless oil (10.23 mg, 34.82%). ¹H NMR (500 MHz, CDCl₃) δ 8.78 (s, 1H), 8.32 (d, J=5.8 Hz, 1H), 7.98 (s, 1H), 6.75 (d, J=2.4 Hz, 1H), 6.49 (dd, J=5.9, 2.4 Hz, 1H), 5.93 (tt, J=7.9, 6.6 Hz, 1H), 5.28 (t, J=6.6 Hz, 2H), 5.10-4.99 (m, 2H), 4.26-4.15 (m, 4H), 3.63 (s, 2H), 3.51 (t, J=6.8 Hz, 2H), 2.37 (t, J=6.9 Hz, 2H). MS m/z: 432 [M+H]⁺.

### Example 377: 6-[1-(2,2-difluoroethyl)-1H-pyrazolo[3,4-b]pyrazin-6-yl]-2-[4-(trifluoromethyl)benzoyl]-2,6-diazaspiro[3.4]octane

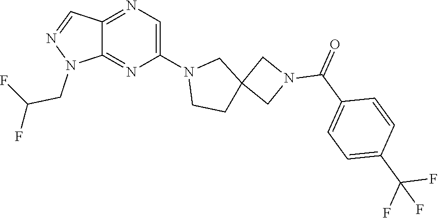

Followed the General procedure A to afford the desired product as a white wax (18 mg, 64%). ¹H NMR (500 MHz, CDCl₃) δ 8.06 (s, 1H), 7.95 (s, 1H), 7.79-7.76 (m, 2H), 7.69 (d, J=8.2 Hz, 2H), 6.22 (tt, J=55.6, 4.5 Hz, 1H), 4.66 (td, J=13.4, 4.5 Hz, 2H), 4.35 (d, J=8.9 Hz, 1H), 4.32-4.20 (m, 3H), 3.87 (s, 2H), 3.79-3.64 (m, 2H), 2.36 (q, J=7.1 Hz, 2H). MS m/z: 467 [M+H]⁺.

### Example 378: 2-(4-chlorobenzoyl)-6-[1-(2,2-difluoroethyl)-1H-pyrazolo[3,4-b]pyrazin-6-yl]-2,6-diazaspiro[3.4]octane

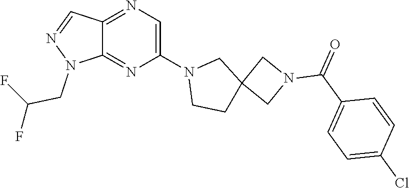 [SMILES: O=C(c1ccc(Cl)cc1)N1CC2(CCN(c3cnc4cnn(CC(F)F)c4n3)C2)C1]

Followed the General procedure A to afford the desired product as a white wax (20 mg, 76%). ¹H NMR (500 MHz, CDCl₃) δ 8.05 (s, 1H), 7.95 (s, 1H), 7.64-7.59 (m, 2H), 7.43-7.37 (m, 2H), 6.22 (tt, J=55.6, 4.5 Hz, 1H), 4.66 (td, J=13.3, 4.5 Hz, 2H), 4.35 (d, J=8.8 Hz, 1H), 4.25 (dd, J=16.4, 8.5 Hz, 3H), 3.86 (d, J=2.5 Hz, 2H), 3.71 (dt, J=23.3, 6.9 Hz, 2H), 2.35 (q, J=7.0 Hz, 2H). MS m/z: 433 [M+H]⁺.

### Example 379: 6-[1-(2,2-difluoroethyl)-1H-pyrazolo[3,4-b]pyrazin-6-yl]-2-(4-methoxybenzoyl)-2,6-diazaspiro[3.4]octane

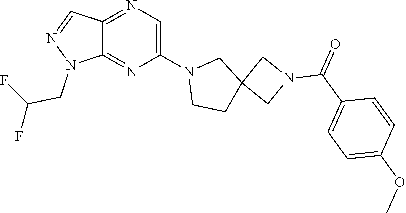 [SMILES: COc1ccc(C(=O)N2CC3(CCN(c4cnc5cnn(CC(F)F)c5n4)C3)C2)cc1]

Followed the General procedure A to afford the desired product as a off-white wax (21 mg, 81%). ¹H NMR (500 MHz, CDCl₃) δ 8.05 (s, 1H), 7.95 (s, 1H), 7.68-7.62 (m, 2H), 6.94-6.89 (m, 2H), 6.22 (tt, J=55.6, 4.5 Hz, 1H), 4.66 (td, J=13.4, 4.5 Hz, 2H), 4.42-4.18 (m, 4H), 3.84 (s, 5H), 3.77-3.62 (m, 2H), 2.34 (q, J=6.5 Hz, 2H). MS m/z: 429 [M+H]⁺.

### Example 380: 6-[1-(2,2-difluoroethyl)-1H-pyrazolo[3,4-b]pyrazin-6-yl]-2-(3-methoxybenzoyl)-2,6-diazaspiro[3.4]octane

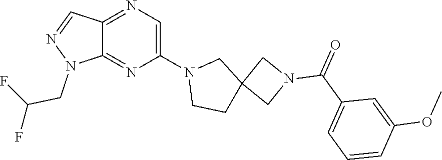 [SMILES: COc1cccc(C(=O)N2CC3(CCN(c4cnc5cnn(CC(F)F)c5n4)C3)C2)c1]

Followed the General procedure A to afford the desired product as a white wax (19 mg, 73%). ¹H NMR (500 MHz, CDCl₃) δ 8.05 (s, 1H), 7.94 (s, 1H), 7.32 (t, J=7.9 Hz, 1H), 7.22 (dd, J=2.7, 1.5 Hz, 1H), 7.19 (dt, J=7.6, 1.2 Hz, 1H), 7.01 (ddd, J=8.3, 2.6, 1.0 Hz, 1H), 6.22 (tt, J=55.6, 4.5 Hz, 1H), 4.66 (td, J=13.4, 4.5 Hz, 2H), 4.36-4.20 (m, 4H), 3.84 (s, 5H), 3.70 (dt, J=25.1, 6.9 Hz, 2H), 2.34 (q, J=7.1 Hz, 2H). MS m/z: 429 [M+H]⁺.

### Example 381: 6-[1-(2,2-difluoroethyl)-1H-pyrazolo[3,4-b]pyrazin-6-yl]-2-(3-methylbenzoyl)-2,6-diazaspiro[3.4]octane

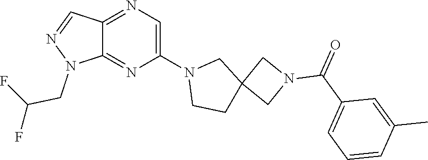 [SMILES: Cc1cccc(C(=O)N2CC3(CCN(c4cnc5cnn(CC(F)F)c5n4)C3)C2)c1]

Followed the General procedure A to afford the desired product as a white wax (17 mg, 68%). ¹H NMR (500 MHz, CDCl₃) δ 8.05 (s, 1H), 7.94 (s, 1H), 7.49 (d, J=1.4 Hz, 1H), 7.43-7.40 (m, 1H), 7.32-7.27 (m, 2H), 6.22 (tt, J=55.6, 4.5 Hz, 1H), 4.66 (td, J=13.4, 4.5 Hz, 2H), 4.38-4.19 (m, 4H), 3.85 (d, J=4.1 Hz, 2H), 3.70 (dt, J=25.8, 7.1 Hz, 2H), 2.38 (s, 3H), 2.34 (q, J=6.9 Hz, 2H). MS m/z: 413 [M+H]⁺.

### Example 382: 6-[1-(2,2-difluoroethyl)-1H-pyrazolo[3,4-b]pyrazin-6-yl]-2-[3-(trifluoromethyl)benzoyl]-2,6-diazaspiro[3.4]octane

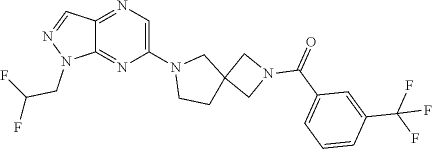 [SMILES: O=C(c1cccc(C(F)(F)F)c1)N1CC2(CCN(c3cnc4cnn(CC(F)F)c4n3)C2)C1]

Followed the General procedure A to afford the desired product as a white wax (20 mg, 71%). ¹H NMR (500 MHz, CDCl₃) δ 8.05 (s, 1H), 7.95 (s, 1H), 7.92 (d, J=1.8 Hz, 1H), 7.86 (dt, J=7.8, 1.4 Hz, 1H), 7.74 (dt, J=8.0, 1.2 Hz, 1H), 7.57 (t, J=7.8 Hz, 1H), 6.22 (tt, J=55.6, 4.5 Hz, 1H), 4.66 (td, J=13.4, 4.5 Hz, 2H), 4.37 (d, J=8.8 Hz, 1H), 4.27 (q, J=10.1 Hz, 3H), 3.88 (d, J=3.1 Hz, 2H), 3.72 (dt, J=21.8, 6.9 Hz, 2H), 2.36 (q, J=6.6 Hz, 2H). MS m/z: 467 [M+H]⁺.

### Example 383: 2-(2-chlorobenzoyl)-6-[1-(2,2-difluoroethyl)-1H-pyrazolo[3,4-b]pyrazin-6-yl]-2,6-diazaspiro[3.4]octane

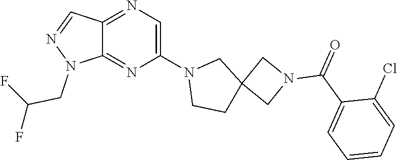 [SMILES: O=C(c1ccccc1Cl)N1CC2(CCN(c3cnc4cnn(CC(F)F)c4n3)C2)C1]

Followed the General procedure A to afford the desired product as a white wax (20 mg, 76%). ¹H NMR (500 MHz, CDCl₃) δ 8.05 (s, 1H), 7.94 (s, 1H), 7.42-7.29 (m, 4H), 6.22 (tt, J=55.6, 4.5 Hz, 1H), 4.66 (td, J=13.4, 4.5 Hz, 2H), 4.31-4.18 (m, 2H), 4.05-3.90 (m, 2H), 3.83 (s, 2H), 3.73 (ddd, J=10.7, 7.5, 5.9 Hz, 1H), 3.65 (dt, J=10.7, 7.1 Hz, 1H), 2.40-2.27 (m, 2H). MS m/z: 433 [M+H]⁺.

### Example 384: 6-[1-(2,2-difluoroethyl)-1H-pyrazolo[3,4-b]pyrazin-6-yl]-2-(2-methylbenzoyl)-2,6-diazaspiro[3.4]octane

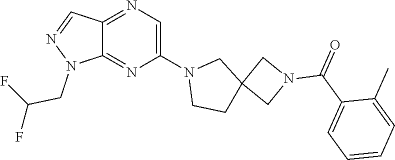 [SMILES: Cc1ccccc1C(=O)N1CC2(CCN(c3cnc4cnn(CC(F)F)c4n3)C2)C1]

Followed the General procedure A to afford the desired product as a off-white wax (22 mg, 88%). ¹H NMR (500 MHz, CDCl₃) δ 8.05 (s, 1H), 7.93 (s, 1H), 7.33-7.26 (m, 2H), 7.25-7.16 (m, 2H), 6.22 (tt, J=55.6, 4.5 Hz, 1H), 4.66 (td, J=13.4, 4.5 Hz, 2H), 4.22 (q, J=10.2 Hz, 2H), 4.02-3.86 (m, 2H), 3.83 (t, J=8.1 Hz, 2H), 3.76-3.68 (m, 1H), 3.64 (dt, J=10.6, 7.0 Hz, 1H), 2.43 (s, 3H), 2.31 (ddt, J=25.9, 12.7, 6.6 Hz, 2H). MS m/z: 413 [M+H]⁺.

### Example 385: 6-[1-(2,2-difluoroethyl)-1H-pyrazolo[3,4-b]pyrazin-6-yl]-2-(4-methylbenzenesulfonyl)-2,6-diazaspiro[3.4]octane

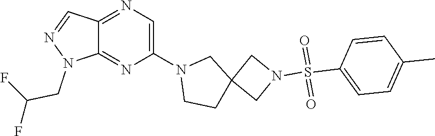

Followed the General procedure A to afford the desired product as a white wax (19 mg, 70%). ¹H NMR (500 MHz, CDCl₃) δ 8.03 (s, 1H), 7.85 (s, 1H), 7.78-7.74 (m, 2H), 7.43-7.40 (m, 2H), 6.20 (tt, J=55.6, 4.5 Hz, 1H), 4.63 (td, J=13.3, 4.5 Hz, 2H), 3.79 (s, 4H), 3.59 (t, J=6.9 Hz, 2H), 3.56 (s, 2H), 2.49 (s, 3H), 2.13 (t, J=6.9 Hz, 2H). MS m/z: 449 [M+H]⁺.

### Example 386: 2-(4-chlorobenzenesulfonyl)-6-[1-(2,2-difluoroethyl)-1H-pyrazolo[3,4-b]pyrazin-6-yl]-2,6-diazaspiro[3.4]octane

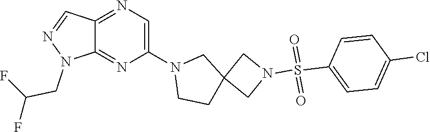

Followed the General procedure A to afford the desired product as a white wax (26 mg, 78%). ¹H NMR (500 MHz, CDCl₃) δ 8.04 (s, 1H), 7.88 (s, 1H), 7.84-7.80 (m, 2H), 7.61-7.57 (m, 2H), 6.20 (tt, J=55.6, 4.4 Hz, 1H), 4.64 (td, J=13.3, 4.5 Hz, 2H), 3.83-3.79 (m, 4H), 3.64-3.59 (m, 4H), 2.17 (t, J=6.9 Hz, 2H). MS m/z: 469 [M+H]⁺.

### Example 387: 6-[1-(2,2-difluoroethyl)-1H-pyrazolo[3,4-b]pyrazin-6-yl]-2-[4-(difluoromethoxy)benzoyl]-2,6-diazaspiro[3.4]octane

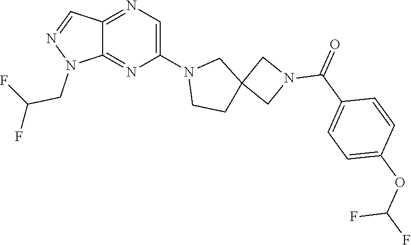 [SMILES: O=C(c1ccc(OC(F)F)cc1)N1CC2(CCN(c3cnc4cnn(CC(F)F)c4n3)C2)C1]

Followed the General procedure A to afford the desired product as a white wax (18 mg, 64%). ¹H NMR (500 MHz, CDCl₃) δ 8.05 (s, 1H), 7.95 (s, 1H), 7.72-7.66 (m, 2H), 7.18-7.12 (m, 2H), 6.56 (t, J=73.1 Hz, 1H), 6.22 (tt, J=55.6, 4.5 Hz, 1H), 4.66 (td, J=13.4, 4.5 Hz, 2H), 4.41-4.19 (m, 4H), 3.86 (d, J=2.6 Hz, 2H), 3.79-3.62 (m, 2H), 2.35 (q, J=6.9 Hz, 2H). MS m/z: 465 [M+H]⁺.

### Example 388: 6-[1-(2,2-difluoroethyl)-1H-pyrazolo[3,4-b]pyrazin-6-yl]-2-[3-(trifluoromethoxy)benzoyl]-2,6-diazaspiro[3.4]octane

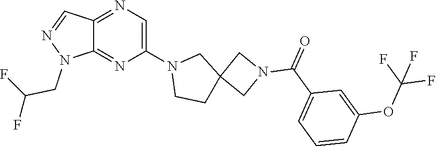 [SMILES: O=C(c1cccc(OC(F)(F)F)c1)N1CC2(CCN(c3cnc4cnn(CC(F)F)c4n3)C2)C1]

Followed the General procedure A to afford the desired product as a colorless oil (19 mg, 65.14%). ¹H NMR (500 MHz, CDCl₃) δ 8.06 (s, 1H), 7.95 (s, 1H), 7.60 (dt, J=7.8, 1.2 Hz, 1H), 7.52 (dt, J=2.5, 1.2 Hz, 1H), 7.47 (t, J=8.0 Hz, 1H), 7.34 (ddt, J=8.2, 2.4, 1.0 Hz, 1H), 6.22 (tt, J=55.6, 4.5 Hz, 1H), 4.66 (td, J=13.4, 4.5 Hz, 2H), 4.42-4.19 (m, 4H), 3.87 (d, J=2.3 Hz, 2H), 3.71 (dt, J=21.2, 6.8 Hz, 2H), 2.36 (q, J=6.6 Hz, 2H). MS m/z: 483 [M+H]⁺.

### Example 389: 6-[1-(2,2-difluoroethyl)-1H-pyrazolo[3,4-b]pyrazin-6-yl]-2-[2-(difluoromethoxy)benzoyl]-2,6-diazaspiro[3.4]octane

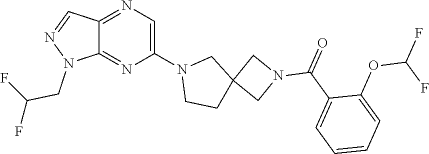 [SMILES: O=C(c1ccccc1OC(F)F)N1CC2(CCN(c3cnc4cnn(CC(F)F)c4n3)C2)C1]

Followed the General procedure A to afford the desired product as a colorless oil (19 mg, 67.66%). ¹H NMR (500 MHz, CDCl₃) δ 8.05 (s, 1H), 7.94 (s, 1H), 7.51-7.42 (m, 2H), 7.30 (td, J=7.6, 1.0 Hz, 1H), 7.20 (dd, J=8.2, 1.1 Hz, 1H), 6.59 (t, J=74.0 Hz, 1H), 6.22 (tt, J=55.6, 4.5 Hz, 1H), 4.66 (td, J=13.3, 4.5 Hz, 2H), 4.27-4.17 (m, 2H), 4.06 (d, J=9.0 Hz, 1H), 4.01-3.97 (m, 1H), 3.83 (s, 2H), 3.73 (ddd, J=10.7, 7.5, 5.9 Hz, 1H), 3.65 (dt, J=10.8, 7.1 Hz, 1H), 2.39-2.27 (m, 2H). MS m/z: 465 [M+H]⁺.

### Example 390: 6-[1-(2,2-difluoroethyl)-1H-pyrazolo[3,4-b]pyrazin-6-yl]-2-[2-(trifluoromethoxy)benzoyl]-2,6-diazaspiro[3.4]octane

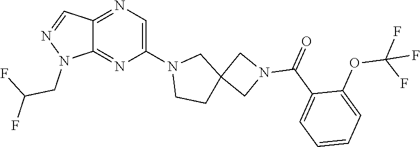 [SMILES: O=C(c1ccccc1OC(F)(F)F)N1CC2(CCN(c3cnc4cnn(CC(F)F)c4n3)C2)C1]

Followed the General procedure A to afford the desired product as a colorless oil (20 mg, 69%). ¹H NMR (500 MHz, CDCl₃) δ 8.05 (s, 1H), 7.94 (s, 1H), 7.53 (dd, J=7.6, 1.8 Hz, 1H), 7.48 (ddd, J=8.3, 7.5, 1.8 Hz, 1H), 7.38 (td, J=7.5, 1.1 Hz, 1H), 7.31 (dt, J=8.3, 1.4 Hz, 1H), 6.22 (tt, J=55.6, 4.5 Hz, 1H), 4.66 (td, J=13.4, 4.5 Hz, 2H), 4.29-4.18 (m, 2H), 4.05 (d, J=9.0 Hz, 1H), 4.00-3.95 (m, 1H), 3.82 (s, 2H), 3.73 (ddd, J=10.7, 7.5, 5.8 Hz, 1H), 3.65 (dt, J=10.8, 7.1 Hz, 1H), 2.40-2.26 (m, 2H). MS m/z: 483 [M+H]⁺.

### Example 391: 6-[1-(2,2-difluoroethyl)-1H-pyrazolo[3,4-b]pyrazin-6-yl]-2-(2-methoxybenzoyl)-2,6-diazaspiro[3.4]octane

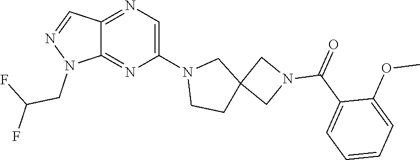 [SMILES: COc1ccccc1C(=O)N1CC2(CCN(c3cnc4cnn(CC(F)F)c4n3)C2)C1]

Followed the General procedure A to afford the desired product as a colorless oil (19 mg, 73%). ¹H NMR (500 MHz, CDCl₃) δ 8.05 (s, 1H), 7.94 (s, 1H), 7.42-7.35 (m, 2H), 7.00 (td, J=7.5, 1.0 Hz, 1H), 6.93 (d, J=8.3 Hz, 1H), 6.22 (tt, J=55.6, 4.5 Hz, 1H), 4.66 (tdd, J=13.5, 4.5, 1.5 Hz, 2H), 4.22 (q, J=10.2 Hz, 2H), 4.03 (d, J=9.1 Hz, 1H), 3.93 (d, J=9.0 Hz, 1H), 3.89 (s, 3H), 3.82 (s, 2H), 3.77-3.68 (m, 1H), 3.64 (dt, J=10.7, 7.1 Hz, 1H), 2.31 (ddt, J=25.8, 12.7, 6.5 Hz, 2H). MS m/z: 429 [M+H]⁺.

### Example 392: 6-[1-(2,2-difluoroethyl)-1H-pyrazolo[3,4-b]pyrazin-6-yl]-2-[2-(trifluoromethyl)benzoyl]-2,6-diazaspiro[3.4]octane

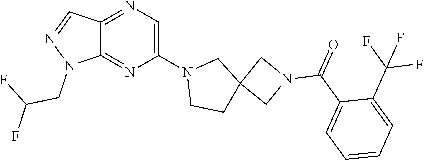 [SMILES: O=C(c1ccccc1C(F)(F)F)N1CC2(CCN(c3cnc4cnn(CC(F)F)c4n3)C2)C1]

Followed the General procedure A to afford the desired product as a colorless oil (15 mg, 53%). ¹H NMR (500 MHz, CDCl₃) δ 8.05 (s, 1H), 7.93 (s, 1H), 7.73 (d, J=7.8 Hz, 1H), 7.60 (td, J=7.5, 1.3 Hz, 1H), 7.57-7.51 (m, 1H), 7.42 (d, J=7.5 Hz, 1H), 6.22 (tt, J=55.6, 4.5 Hz, 1H), 4.67 (td, J=13.4, 4.5 Hz, 2H), 4.23 (q, J=10.3 Hz, 2H), 3.92 (d, J=9.0 Hz, 1H), 3.84 (d, J=4.9 Hz, 1H), 3.83-3.80 (m, 2H), 3.72 (ddd, J=10.7, 7.6, 5.8 Hz, 1H), 3.63 (dt, J=10.8, 7.1 Hz, 1H), 2.46-2.22 (m, 2H). MS m/z: 467 [M+H]⁺.

### Example 393: 2-(2-chlorobenzenesulfonyl)-6-[1-(2,2-difluoroethyl)-1H-pyrazolo[3,4-b]pyrazin-6-yl]-2,6-diazaspiro[3.4]octane

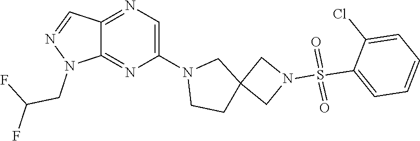 [SMILES: O=S(=O)(c1ccccc1Cl)N1CC2(CCN(c3cnc4cnn(CC(F)F)c4n3)C2)C1]

Followed the General procedure A to afford the desired product as a colorless oil (18 mg, 63.49%). ¹H NMR (500 MHz, CDCl₃) δ 8.06 (dd, J=7.9, 1.6 Hz, 1H), 8.05 (s, 1H), 7.92 (s, 1H), 7.61-7.50 (m, 2H), 7.42 (ddd, J=7.9, 7.2, 1.5 Hz, 1H), 6.22 (tt, J=55.6, 4.5 Hz, 1H), 4.66 (td, J=13.3, 4.5 Hz, 2H), 4.07 (q, J=8.0 Hz, 4H), 3.77 (s, 2H), 3.67 (t, J=6.9 Hz, 2H), 2.30 (t, J=6.9 Hz, 2H). MS m/z: 469 [M+H]⁺.

### Example 394: 6-[1-(2,2-difluoroethyl)-1H-pyrazolo[3,4-b]pyrazin-6-yl]-2-(2-methylbenzenesulfonyl)-2,6-diazaspiro[3.4]octane

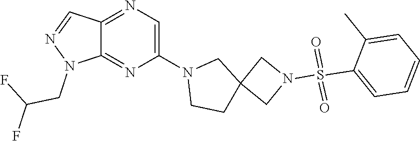 [SMILES: Cc1ccccc1S(=O)(=O)N1CC2(CCN(c3cnc4cnn(CC(F)F)c4n3)C2)C1]

Followed the General procedure A to afford the desired product as a colorless oil (2 mg, 22%). ¹H NMR (500 MHz, CDCl₃) δ 8.03 (d, J=4.2 Hz, 1H), 7.96 (dd, J=7.9, 1.4 Hz, 1H), 7.91 (s, 1H), 7.50 (td, J=7.5, 1.4 Hz, 1H), 7.37-7.31 (m, 2H), 6.21 (tt, J=55.6, 4.5 Hz, 1H), 4.65 (td, J=13.4, 4.5 Hz, 2H), 3.94-3.83 (m, 4H), 3.82-3.70 (m, 3H), 3.70-3.52 (m, 3H), 2.67 (s, 3H), 2.27 (t, J=6.9 Hz, 2H). MS m/z: 449 [M+H]⁺.

### Example 395: 6-[1-(2,2-difluoroethyl)-1H-pyrazolo[3,4-b]pyrazin-6-yl]-2-(2-fluorobenzenesulfonyl)-2,6-diazaspiro[3.4]octane

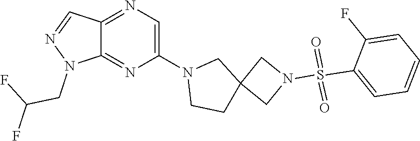 [SMILES: O=S(=O)(c1ccccc1F)N1CC2(CCN(c3cnc4cnn(CC(F)F)c4n3)C2)C1]

Followed the General procedure A to afford the desired product as a white wax (15 mg, 54.83%). ¹H NMR (500 MHz, CDCl₃) δ 8.04 (s, 1H), 7.92-7.87 (m, 2H), 7.69-7.63 (m, 1H), 7.34 (td, J=7.6, 1.1 Hz, 1H), 7.30 (ddd, J=9.6, 8.3, 1.1 Hz, 1H), 6.21 (tt, J=55.6, 4.5 Hz, 1H), 4.65 (td, J=13.3, 4.5 Hz, 2H), 4.03-3.95 (m, 4H), 3.69 (s, 2H), 3.65 (t, J=6.9 Hz, 2H), 2.25 (t, J=6.9 Hz, 2H). MS m/z: 453 [M+H]⁺.

### Example 396: 6-[1-(2,2-difluoroethyl)-1H-pyrazolo[3,4-b]pyrazin-6-yl]-2-[2-(trifluoromethyl)benzenesulfonyl]-2,6-diazaspiro[3.4]octane

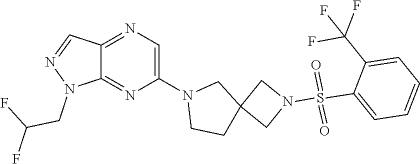 [SMILES: O=S(=O)(c1ccccc1C(F)(F)F)N1CC2(CCN(c3cnc4cnn(CC(F)F)c4n3)C2)C1]

Followed the General procedure A to afford the desired product as a white wax (19 mg, 62.54%). ¹H NMR (500 MHz, CDCl₃) δ 8.27-8.16 (m, 1H), 8.05 (s, 1H), 7.98-7.86 (m, 2H), 7.82-7.67 (m, 2H), 6.21 (tt, J=55.6, 4.5 Hz, 1H), 4.66 (td, J=13.3, 4.5 Hz, 2H), 4.02 (q, J=7.9 Hz, 4H), 3.76 (s, 2H), 3.67 (t, J=6.9 Hz, 2H), 2.30 (t, J=6.9 Hz, 2H). MS m/z: 503 [M+H]⁺.

### Example 397: 8-[1-(2,2-difluoroethyl)-1H-pyrazolo[3,4-b]pyrazin-6-yl]-2-[(5-fluoropyridin-2-yl)methyl]-2,8-diazaspiro[4.5]decan-3-one

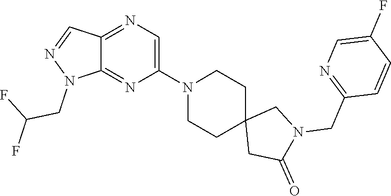 [SMILES: O=C1CC2(CCN(c3cnc4cnn(CC(F)F)c4n3)CC2)CN1Cc1ccc(F)cn1]

Followed the General procedure A to afford the desired product as a white wax (22 mg, 55.37%). ¹H NMR (500 MHz, CDCl₃) δ 8.40 (d, J=2.9 Hz, 1H), 8.22 (s, 1H), 8.03 (s, 1H), 7.41 (td, J=8.3, 2.9 Hz, 1H), 7.32 (dd, J=8.6, 4.4 Hz, 1H), 6.20 (tt, J=55.6, 4.5 Hz, 1H), 4.65 (td, J=13.3, 4.5 Hz, 2H), 4.59 (s, 2H), 3.83 (ddd, J=13.8, 6.7, 4.1 Hz, 2H), 3.65 (ddd, J=13.7, 7.9, 4.0 Hz, 2H), 3.31 (s, 2H), 2.45 (s, 2H), 1.80-1.70 (m, 4H). MS m/z: 446 [M+H]⁺.

### Example 398: 8-[1-(2,2-difluoroethyl)-1H-pyrazolo[3,4-b]pyrazin-6-yl]-2-[(5-methyl-1,2-oxazol-3-yl)methyl]-2,8-diazaspiro[4.5]decan-3-one

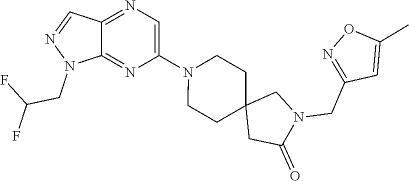 [SMILES: Cc1cc(CN2CC3(CCN(c4cnc5cnn(CC(F)F)c5n4)CC3)CC2=O)no1]

Followed the General procedure A to afford the desired product as a colorless oil (14 mg, 36.38%). ¹H NMR (500 MHz, CDCl₃) δ 8.22 (s, 1H), 8.04 (s, 1H), 6.20 (tt, J=55.6, 4.5 Hz, 1H), 5.96 (d, J=1.0 Hz, 1H), 4.70-4.62 (m, 2H), 4.50 (s, 2H), 3.83 (dt, J=13.8, 5.0 Hz, 2H), 3.65 (ddd, J=13.9, 7.3, 4.6 Hz, 2H), 3.27 (s, 2H), 2.46-2.40 (m, 5H), 1.74 (dq, J=7.7, 3.1 Hz, 4H). MS m/z: 432 [M+H]⁺.

### Example 399: 8-[1-(2,2-difluoroethyl)-1H-pyrazolo[3,4-b]pyrazin-6-yl]-2-[(4-methyl-1,3-thiazol-2-yl)methyl]-2,8-diazaspiro[4.5]decan-3-one

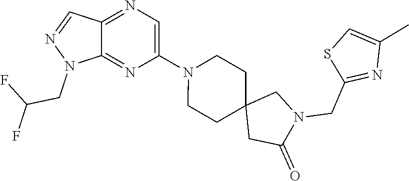 [SMILES: Cc1csc(CN2CC3(CCN(c4cnc5cnn(CC(F)F)c5n4)CC3)CC2=O)n1]

Followed the General procedure A to afford the desired product as a light yellow wax (14 mg, 35.07%). ¹H NMR (500 MHz, CDCl₃) δ 8.22 (s, 1H), 8.04 (s, 1H), 6.87 (q, J=1.0 Hz, 1H), 6.20 (tt, J=55.6, 4.5 Hz, 1H), 4.77 (s, 2H), 4.65 (td, J=13.3, 4.5 Hz, 2H), 3.82 (ddd, J=13.8, 6.6, 4.4 Hz, 2H), 3.67 (ddd, J=13.8, 7.6, 4.3 Hz, 2H), 3.34 (s, 2H), 2.48-2.41 (m, 5H), 1.82-1.71 (m, 4H). MS m/z: 448 [M+H]⁺.

### Example 400: 2-[(5-chloropyridin-2-yl)methyl]-8-[1-(2,2-difluoroethyl)-1H-pyrazolo[3,4-b]pyrazin-6-yl]-2,8-diazaspiro[4.5]decan-3-one

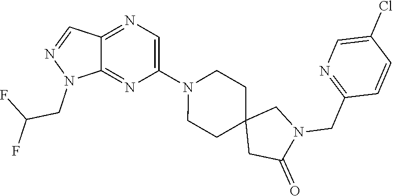 [SMILES: O=C1CC2(CCN(c3cnc4cnn(CC(F)F)c4n3)CC2)CN1Cc1ccc(Cl)cn1]

Followed the General procedure A to afford the desired product as a colorless oil (4.7 mg, 17.11%). ¹H NMR (500 MHz, CDCl₃) δ 8.50 (dd, J=2.5, 0.7 Hz, 1H), 8.24 (s, 1H), 8.03 (s, 1H), 7.65 (dd, J=8.3, 2.5 Hz, 1H), 7.21 (dd, J=8.3, 0.7 Hz, 1H), 6.21 (tt, J=55.6, 4.5 Hz, 1H), 4.65 (td, J=13.3, 4.5 Hz, 2H), 4.58 (s, 2H), 4.34 (dt, J=13.6, 4.7 Hz, 2H), 3.50-3.36 (m, 4H), 2.12-1.98 (m, 4H), 1.61-1.55 (m, 2H). MS m/z: 462 [M+H]⁺.

### Example 401: 2-(5-chloropyridin-3-yl)-6-[1-(2,2-difluoroethyl)-1H-pyrazolo[3,4-b]pyrazin-6-yl]-2,6-diazaspiro[3.4]octane

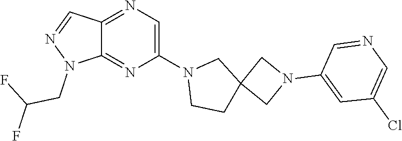 [SMILES: FC(F)Cn1ncc2ncc(N3CCC4(CN(c5cncc(Cl)c5)C4)C3)nc21]

Followed the General procedure A to afford the desired product as a white wax (4.2 mg, 11.41%). ¹H NMR (500 MHz, CDCl₃) δ 8.06 (s, 1H), 7.99 (d, J=2.1 Hz, 1H), 7.97 (s, 1H), 7.76 (d, J=2.5 Hz, 1H), 6.74 (t, J=2.2 Hz, 1H), 6.22 (tt, J=55.6, 4.5 Hz, 1H), 4.67 (td, J=13.4, 4.5 Hz, 2H), 4.03-3.95 (m, 4H), 3.88 (s, 2H), 3.74 (t, J=6.9 Hz, 2H), 2.39 (t, J=6.9 Hz, 2H). MS m/z: 406 [M+H]⁺.

### Example 402: 2-(5-chloro-4-methylpyrimidin-2-yl)-6-[1-(2,2-difluoroethyl)-1H-pyrazolo[3,4-b]pyrazin-6-yl]-2,6-diazaspiro[3.4]octane

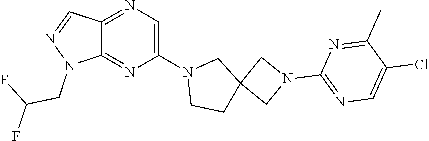 [SMILES: Cc1nc(N2CC3(CCN(c4cnc5cnn(CC(F)F)c5n4)C3)C2)ncc1Cl]

Followed the General procedure A to afford the desired product as a white foam (25 mg, 65.5%). ¹H NMR (500 MHz, CDCl₃) δ 8.16 (s, 1H), 8.05 (s, 1H), 7.96 (s, 1H), 6.23 (tt, J=55.7, 4.5 Hz, 1H), 4.66 (td, J=13.3, 4.5 Hz, 2H), 4.18-4.09 (m, 4H), 3.86 (s, 2H), 3.73 (t, J=6.9 Hz, 2H), 2.44 (s, 3H), 2.36 (t, J=6.9 Hz, 2H). MS m/z: 421 [M+H]⁺.

### Example 403: 2-(5-chloropyridin-2-yl)-6-[1-(2,2-difluoroethyl)-1H-pyrazolo[3,4-b]pyrazin-6-yl]-2,6-diazaspiro[3.4]octane

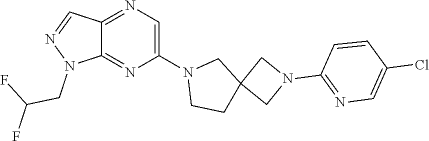 [SMILES: FC(F)Cn1ncc2ncc(N3CCC4(CN(c5ccc(Cl)cn5)C4)C3)nc21]

Followed the General procedure A to afford the desired product as a off-white wax (17 mg, 46.18%). ¹H NMR (500 MHz, CDCl₃) δ 8.10 (dd, J=2.6, 0.7 Hz, 1H), 8.05 (s, 1H), 7.96 (s, 1H), 7.43 (dd, J=8.8, 2.6 Hz, 1H), 6.27 (dd, J=8.8, 0.7 Hz, 1H), 6.22 (tt, J=55.7, 4.5 Hz, 1H), 4.66 (td, J=13.4, 4.5 Hz, 2H), 4.10-3.96 (m, 4H), 3.87 (s, 2H), 3.73 (t, J=6.9 Hz, 2H), 2.37 (t, J=6.8 Hz, 2H). MS m/z: 406 [M+H]⁺.

### Example 404: 6-[1-(2,2-difluoroethyl)-1H-pyrazolo[3,4-b]pyrazin-6-yl]-2-[6-methyl-2-(trifluoromethyl)pyrimidin-4-yl]-2,6-diazaspiro[3.4]octane

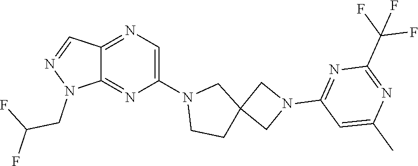 [SMILES: Cc1cc(N2CC3(CCN(c4cnc5cnn(CC(F)F)c5n4)C3)C2)nc(C(F)(F)F)n1]

Followed the General procedure A to afford the desired product as a white wax (28 mg, 67.94%). ¹H NMR (500 MHz, CDCl₃) δ 8.06 (s, 1H), 7.96 (s, 1H), 6.22 (tt, J=55.7, 4.5 Hz, 1H), 6.15 (s, 1H), 4.66 (td, J=13.4, 4.5 Hz, 2H), 4.29-4.02 (m, 4H), 3.89 (s, 2H), 3.75 (t, J=6.8 Hz, 2H), 2.47-2.41 (m, 3H), 2.39 (t, J=6.9 Hz, 2H). MS m/z: 455 [M+H]⁺.

### Example 405: 6-[1-(2,2-difluoroethyl)-1H-pyrazolo[3,4-b]pyrazin-6-yl]-2-[6-(trifluoromethoxy)pyridin-2-yl]-2,6-diazaspiro[3.4]octane

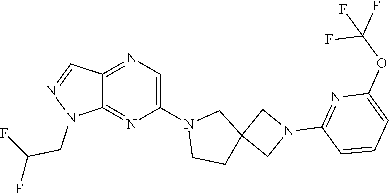 [SMILES: FC(F)Cn1ncc2ncc(N3CCC4(CN(c5cccc(OC(F)(F)F)n5)C4)C3)nc21]

Followed the General procedure A to afford the desired product as a colorless oil (5 mg, 12.11%). ¹H NMR (500 MHz, CDCl₃) δ 8.05 (s, 1H), 7.96 (s, 1H), 7.53 (t, J=7.9 Hz, 1H), 6.30 (d, J=3.0 Hz, 1H), 6.22 (tt, J=55.7, 4.5 Hz, 1H), 6.17 (d, J=3.0 Hz, 1H), 4.66 (td, J=13.3, 4.5 Hz, 2H), 4.10-3.99 (m, 4H), 3.86 (s, 2H), 3.73 (t, J=6.9 Hz, 2H), 2.37 (t, J=6.8 Hz, 2H). MS m/z: 456 [M+H]⁺.

### Example 406: 6-[1-(2,2-difluoroethyl)-1H-pyrazolo[3,4-b]pyrazin-6-yl]-2-[6-(difluoromethoxy)pyridin-2-yl]-2,6-diazaspiro[3.4]octane

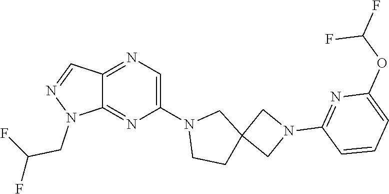 [SMILES: FC(F)Cn1ncc2ncc(N3CCC4(CN(c5cccc(OC(F)F)n5)C4)C3)nc21]

Followed the General procedure A to afford the desired product as a white wax (8 mg, 20.17%). ¹H NMR (500 MHz, CDCl₃) δ 8.05 (s, 1H), 7.96 (s, 1H), 7.57-7.26 (m, 2H), 6.22 (tt, J=55.7, 4.5 Hz, 1H), 6.20 (d, J=3.0 Hz, 1H), 6.05 (d, J=3.0 Hz, 1H), 4.66 (td, J=13.3, 4.5 Hz, 2H), 4.08-3.98 (m, 4H), 3.86 (s, 2H), 3.73 (t, J=6.9 Hz, 2H), 2.36 (t, J=6.9 Hz, 2H). MS m/z: 438 [M+H]⁺.

### Example 407: 2-(5-chloro-3-fluoropyridin-2-yl)-6-[1-(2,2-difluoroethyl)-1H-pyrazolo[3,4-b]pyrazin-6-yl]-2,6-diazaspiro[3.4]octane

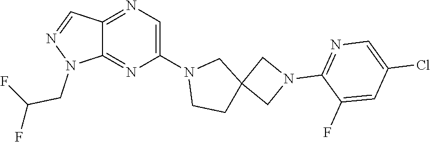 [SMILES: Fc1cc(Cl)cnc1N1CC2(CCN(c3cnc4cnn(CC(F)F)c4n3)C2)C1]

Followed the General procedure A to afford the desired product as a white wax (7.5 mg, 19.51%). ¹H NMR (500 MHz, CDCl₃) δ 8.05 (s, 1H), 7.96 (s, 1H), 7.92 (dd, J=2.1, 0.7 Hz, 1H), 7.22 (dd, J=11.0, 2.1 Hz, 1H), 6.23 (tt, J=55.7, 4.5 Hz, 1H), 4.66 (td, J=13.4, 4.5 Hz, 2H), 4.21-4.12 (m, 4H), 3.87 (s, 2H), 3.73 (t, J=6.9 Hz, 2H), 2.38 (t, J=6.9 Hz, 2H). MS m/z: 424 [M+H]⁺.

### Example 408: 2-(5-chloro-3-methylpyridin-2-yl)-6-[1-(2,2-difluoroethyl)-1H-pyrazolo[3,4-b]pyrazin-6-yl]-2,6-diazaspiro[3.4]octane

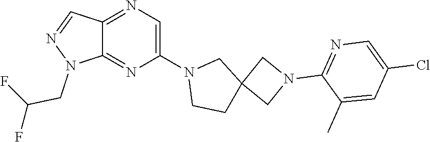 [SMILES: Cc1cc(Cl)cnc1N1CC2(CCN(c3cnc4cnn(CC(F)F)c4n3)C2)C1]

Followed the General procedure A to afford the desired product as a colorless oil (5.5 mg, 14.44%). ¹H NMR (500 MHz, CDCl₃) δ 8.05 (s, 1H), 7.99 (dd, J=2.5, 0.7 Hz, 1H), 7.96 (s, 1H), 6.23 (tt, J=55.6, 4.5 Hz, 1H), 4.67 (td, J=13.4, 4.5 Hz, 2H), 4.15-4.07 (m, 4H), 3.86 (s, 2H), 3.72 (t, J=6.9 Hz, 2H), 2.36 (t, J=6.9 Hz, 2H), 2.19 (t, J=0.8 Hz, 3H). MS m/z: 420 [M+H]⁺.

### Example 409: 6-[1-(2,2-difluoroethyl)-1H-pyrazolo[3,4-b]pyrazin-6-yl]-2-[6-(2,2,2-trifluoroethoxy)pyridin-2-yl]-2,6-diazaspiro[3.4]octane

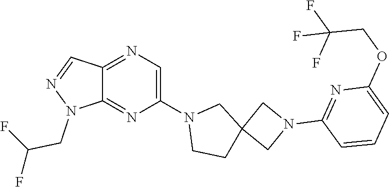 [SMILES: FC(F)Cn1ncc2ncc(N3CCC4(CN(c5cccc(OCC(F)(F)F)n5)C4)C3)nc21]

Followed the General procedure A to afford the desired product as a colorless oil (6 mg, 14.09%). ¹H NMR (500 MHz, CDCl₃) δ 8.05 (s, 1H), 7.96 (s, 1H), 7.44 (t, J=7.9 Hz, 1H), 6.23 (tt, J=55.6, 4.5 Hz, 1H), 6.20 (d, J=7.8 Hz, 1H) 5.94 (dd, J=7.8, 0.6 Hz, 1H), 4.75-4.62 (m, 4H), 4.07-3.97 (m, 4H), 3.86 (s, 2H), 3.73 (t, J=6.9 Hz, 2H), 2.37 (t, J=6.9 Hz, 2H). MS m/z: 470 [M+H]⁺.

### Example 410: 6-[1-(2,2-difluoroethyl)-1H-pyrazolo[3,4-b]pyrazin-6-yl]-2-(2-methoxypyrimidin-5-yl)-2,6-diazaspiro[3.4]octane

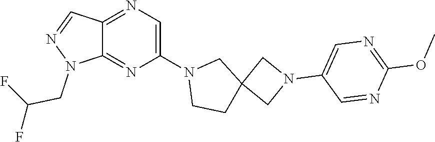 [SMILES: COc1ncc(N2CC3(CCN(c4cnc5cnn(CC(F)F)c5n4)C3)C2)cn1]

Followed the General procedure A to afford the desired product as a colorless oil (8 mg, 21.92%). ¹H NMR (500 MHz, CDCl₃) δ 8.05 (s, 1H), 7.96 (s, 1H), 7.85 (s, 2H), 6.22 (tt, J=55.6, 4.5 Hz, 1H), 4.66 (td, J=13.3, 4.5 Hz, 2H), 3.96-3.91 (m, 7H), 3.88 (s, 2H), 3.73 (t, J=6.9 Hz, 2H), 2.39 (t, J=6.9 Hz, 2H). MS m/z: 403 [M+H]⁺.

### Example 411: 6-[1-(2,2-difluoroethyl)-1H-pyrazolo[3,4-b]pyrazin-6-yl]-2-[2-(trifluoromethoxy)pyridin-4-yl]-2,6-diazaspiro[3.4]octane

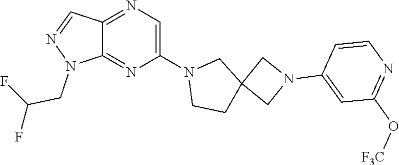 [SMILES: FC(F)Cn1ncc2ncc(N3CCC4(CN(c5ccnc(OC(F)(F)F)c5)C4)C3)nc21]

Followed the General procedure A to afford the desired product as a colorless oil (9 mg, 456.6%). ¹H NMR (500 MHz, CDCl₃) δ 8.06 (s, 1H), 7.99-7.96 (m, 2H), 6.35-6.10 (m, 2H), 5.93 (d, J=2.0 Hz, 1H), 4.67 (td, J=13.3, 4.5 Hz, 2H), 4.07-3.97 (m, 4H), 3.89 (s, 2H), 3.75 (t, J=6.9 Hz, 2H), 2.39 (t, J=6.9 Hz, 2H). MS m/z: 22 [M+H]⁺.

### Example 412: 6-[1-(2,2-difluoroethyl)-1H-pyrazolo[3,4-b]pyrazin-6-yl]-2-[2-(2,2,2-trifluoroethoxy)pyridin-4-yl]-2,6-diazaspiro[3.4]octane

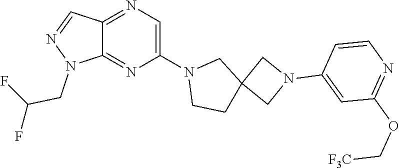 [SMILES: FC(F)Cn1ncc2ncc(N3CCC4(CN(c5ccnc(OCC(F)(F)F)c5)C4)C3)nc21]

Followed the General procedure A to afford the desired product as a colorless oil (6 mg, 470.62%). ¹H NMR (500 MHz, CDCl₃) δ 8.06 (s, 1H), 7.96 (s, 1H), 7.83 (d, J=5.7 Hz, 1H), 6.22 (tt, J=55.7, 4.5 Hz, 1H), 6.08 (dd, J=5.9, 2.1 Hz, 1H), 5.80 (d, J=2.0 Hz, 1H), 4.75-4.63 (m, 4H), 4.02-3.92 (m, 4H), 3.87 (s, 2H), 3.74 (t, J=6.9 Hz, 2H), 2.37 (t, J=6.9 Hz, 2H). MS m/z: 14 [M+H]⁺.

### Example 413: 6-[1-(2,2-difluoroethyl)-1H-pyrazolo[3,4-b]pyrazin-6-yl]-2-{[5-(trifluoromethyl)pyridin-3-yl]sulfonyl}-2,6-diazaspiro[3.4]octane

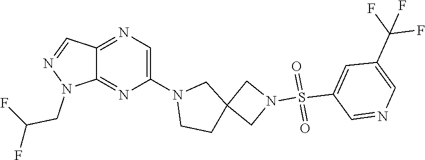 [SMILES: O=S(=O)(c1cncc(C(F)(F)F)c1)N1CC2(CCN(c3cnc4cnn(CC(F)F)c4n3)C2)C1]

Followed the General procedure A to afford the desired product as a white wax (9 mg, 504.55%). ¹H NMR (500 MHz, CDCl₃) δ 9.26 (d, J=2.1 Hz, 1H), 9.19-9.12 (m, 1H), 8.44-8.35 (m, 1H), 8.05 (s, 1H), 7.91 (s, 1H), 6.20 (tt, J=55.6, 4.5 Hz, 1H), 4.65 (td, J=13.3, 4.5 Hz, 2H), 3.95 (q, J=8.1 Hz, 4H), 3.72 (s, 2H), 3.66 (t, J=6.9 Hz, 2H), 2.25 (t, J=6.9 Hz, 2H). MS m/z: 30 [M+H]⁺.

### Example 414: 6-[1-(2,2-difluoroethyl)-1H-pyrazolo[3,4-b]pyrazin-6-yl]-2-{[6-(trifluoromethyl)pyridin-3-yl]sulfonyl}-2,6-diazaspiro[3.4]octane

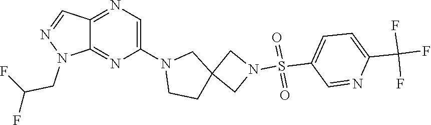 [SMILES: O=S(=O)(c1ccc(C(F)(F)F)nc1)N1CC2(CCN(c3cnc4cnn(CC(F)F)c4n3)C2)C1]

Followed the General procedure A to afford the desired product as a white wax (9.9 mg, 504.54%). ¹H NMR (500 MHz, CDCl₃) δ 9.16 (d, J=2.2 Hz, 1H), 8.34 (dd, J=8.1, 2.2 Hz, 1H), 8.03 (s, 1H), 7.92 (dd, J=8.2, 0.8 Hz, 1H), 7.89 (s, 1H), 6.18 (tt, J=55.5, 4.4 Hz, 1H), 4.62 (td, J=13.4, 4.5 Hz, 2H), 3.92 (q, J=8.1 Hz, 4H), 3.69 (s, 2H), 3.64 (t, J=7.0 Hz, 2H), 2.22 (t, J=6.9 Hz, 2H). MS m/z: 33 [M+H]⁺.

### Example 415: 6-[1-(2,2-difluoroethyl)-1H-pyrazolo[3,4-b]pyrazin-6-yl]-2-(3,3,3-trifluoropropanesulfonyl)-2,6-diazaspiro[3.4]octane

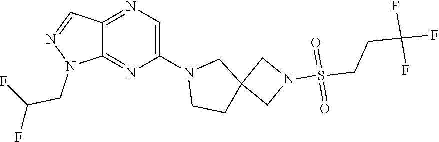 [SMILES: O=S(=O)(CCC(F)(F)F)N1CC2(CCN(c3cnc4cnn(CC(F)F)c4n3)C2)C1]

Followed the General procedure A to afford the desired product as a white wax (9.9 mg, 455.54%). ¹H NMR (500 MHz, CDCl₃) δ 8.06 (s, 1H), 7.95 (s, 1H), 6.22 (tt, J=55.6, 4.5 Hz, 1H), 4.67 (td, J=13.3, 4.5 Hz, 2H), 4.09-3.96 (m, 4H), 3.84 (s, 2H), 3.70 (t, J=6.9 Hz, 2H), 3.26-3.12 (m, 2H), 2.64 (dddd, J=16.7, 14.6, 12.1, 7.2 Hz, 2H), 2.35 (t, J=6.9 Hz, 2H). MS m/z: 36 [M+H]⁺

### Example 416: 6-[1-(2,2-difluoroethyl)-1H-pyrazolo[3,4-b]pyrazin-6-yl]-2-[(6-methylpyridin-3-yl)sulfonyl]-2,6-diazaspiro[3.4]octane

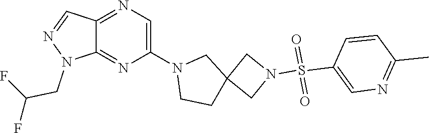 [SMILES: Cc1ccc(S(=O)(=O)N2CC3(CCN(c4cnc5cnn(CC(F)F)c5n4)C3)C2)cn1]

Followed the General procedure A to afford the desired product as a white wax (13 mg, 450.56%). ¹H NMR (500 MHz, CDCl₃) δ 8.97 (d, J=2.3 Hz, 1H), 8.03 (d, J=8.8 Hz, 2H), 7.88 (s, 1H), 7.40 (d, J=8.1 Hz, 1H), 6.20 (tt, J=55.6, 4.5 Hz, 1H), 4.64 (td, J=13.4, 4.5 Hz, 2H), 3.91-3.79 (m, 4H), 3.63 (d, J=7.4 Hz, 4H), 2.71 (s, 3H), 2.19 (t, J=6.9 Hz, 2H). MS m/z: 49 [M+H]⁺.

### Example 417: 6-[1-(2,2-difluoroethyl)-1H-pyrazolo[3,4-b]pyrazin-6-yl]-2-(3-methylbenzenesulfonyl)-2,6-diazaspiro[3.4]octane

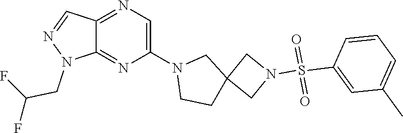 [SMILES: Cc1cccc(S(=O)(=O)N2CC3(CCN(c4cnc5cnn(CC(F)F)c5n4)C3)C2)c1]

Followed the General procedure A to afford the desired product as a white wax (15 mg, 449.65%). ¹H NMR (500 MHz, CDCl₃) δ 8.04 (s, 1H), 7.86 (s, 1H), 7.70-7.65 (m, 2H), 7.51 (dd, J=5.3, 1.1 Hz, 2H), 6.20 (tt, J=55.7, 4.5 Hz, 1H), 4.63 (td, J=13.3, 4.5 Hz, 2H), 3.81 (s, 4H), 3.60 (t, J=6.9 Hz, 2H), 3.55 (s, 2H), 2.48 (s, 3H), 2.14 (t, J=6.9 Hz, 2H). MS m/z: 54 [M+H]⁺.

### Example 418: 2-(3-chlorobenzenesulfonyl)-6-[1-(2,2-difluoroethyl)-1H-pyrazolo[3,4-b]pyrazin-6-yl]-2,6-diazaspiro[3.4]octane

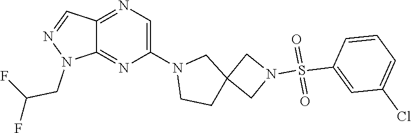 [SMILES: O=S(=O)(c1cccc(Cl)c1)N1CC2(CCN(c3cnc4cnn(CC(F)F)c4n3)C2)C1]

Followed the General procedure to afford the desired product as a white wax (15 mg, 469.56%). ¹H NMR (500 MHz, CDCl₃) δ 8.04 (s, 1H), 7.89-7.86 (m, 2H), 7.76 (dt, J=7.8, 1.4 Hz, 1H), 7.68 (ddd, J=8.0, 2.0, 1.1 Hz, 1H), 7.57 (t, J=7.9 Hz, 1H), 6.20 (tt, J=55.6, 4.5 Hz, 1H), 4.64 (td, J=13.3, 4.5 Hz, 2H), 3.85 (s, 4H), 3.68-3.53 (m, 4H), 2.18 (t, J=7.0 Hz, 2H). MS m/z: 53 [M+H]⁺.

### Example 419: 6-[1-(2,2-difluoroethyl)-1H-pyrazolo[3,4-b]pyrazin-6-yl]-2-(4-methylbenzoyl)-2,6-diazaspiro[3.4]octane

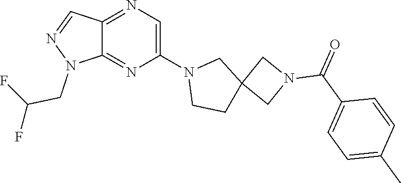 [SMILES: Cc1ccc(C(=O)N2CC3(CCN(c4cnc5cnn(CC(F)F)c5n4)C3)C2)cc1]

Followed the General procedure A to afford the desired product as a colorless oil (36.6 mg, 413.59%). ¹H NMR (500 MHz, CDCl₃) δ 8.05 (s, 1H), 7.94 (s, 1H), 7.56 (d, J=8.2 Hz, 2H), 7.22 (d, J=7.9 Hz, 2H), 6.22 (tt, J=55.6, 4.5 Hz, 1H), 4.66 (td, J=13.3, 4.5 Hz, 2H), 4.39-4.19 (m, 4H), 3.85 (d, J=3.1 Hz, 2H), 3.76-3.64 (m, 2H), 2.39 (s, 3H), 2.33 (q, J=7.1 Hz, 2H). MS m/z: 59 [M+H]⁺.

### Example 420: 2-(4,6-dichloropyridine-2-carbonyl)-6-[1-(2,2-difluoroethyl)-1H-pyrazolo[3,4-b]pyrazin-6-yl]-2,6-diazaspiro[3.4]octane

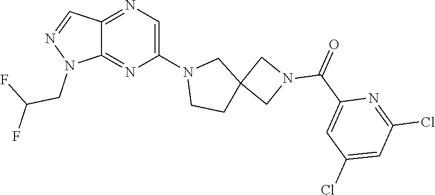 [SMILES: O=C(c1cc(Cl)cc(Cl)n1)N1CC2(CCN(c3cnc4cnn(CC(F)F)c4n3)C2)C1]

Followed the General procedure A to afford the desired product as a white solid (38.4 mg, 468.69%). ¹H NMR (500 MHz, CDCl₃) δ 8.10 (d, J=1.8 Hz, 1H), 8.05 (s, 1H), 7.96 (s, 1H), 7.45 (d, J=1.7 Hz, 1H), 6.22 (tt, J=55.6, 4.5 Hz, 1H), 4.80-4.71 (m, 2H), 4.66 (td, J=13.4, 4.5 Hz, 2H), 4.30-4.19 (m, 2H), 3.94-3.81 (m, 2H), 3.74 (td, J=7.0, 3.9 Hz, 2H), 2.37 (td, J=6.8, 3.7 Hz, 2H). MS m/z: 54 [M+H]⁺.

### Example 421: 2-[(4-chloro-2-fluorophenyl)methyl]-8-[1-(2,2-difluoroethyl)-1H-pyrazolo[3,4-b]pyrazin-6-yl]-2,8-diazaspiro[4.5]decan-3-one

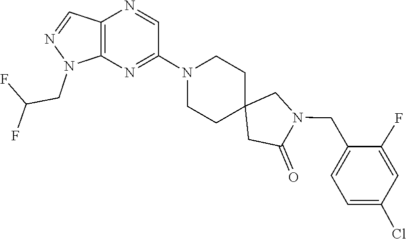 [SMILES: O=C1CC2(CCN(c3cnc4cnn(CC(F)F)c4n3)CC2)CN1Cc1ccc(Cl)cc1F]

Followed the General procedure A to afford the desired product as a colorless oil (44.4 mg, 479.75%). ¹H NMR (500 MHz, CDCl₃) δ 8.21 (s, 1H), 8.03 (s, 1H), 7.27 (t, J=8.1 Hz, 1H), 7.12 (ddd, J=14.4, 8.8, 2.0 Hz, 2H), 6.19 (tt, J=55.6, 4.5 Hz, 1H), 4.64 (td, J=13.3, 4.5 Hz, 2H), 4.50 (d, J=1.2 Hz, 2H), 3.86 (ddd, J=13.8, 6.5, 4.1 Hz, 2H), 3.59 (ddd, J=13.8, 8.4, 3.9 Hz, 2H), 3.17 (s, 2H), 2.42 (s, 2H), 1.70 (dqt, J=13.2, 8.3, 4.0 Hz, 4H). MS m/z: 62 [M+H]⁺.

### Example 422: 6-[1-(2,2-difluoroethyl)-1H-pyrazolo[3,4-b]pyrazin-6-yl]-2-[2-methyl-6-(trifluoromethyl)pyrimidin-4-yl]-2,6-diazaspiro[3.4]octane

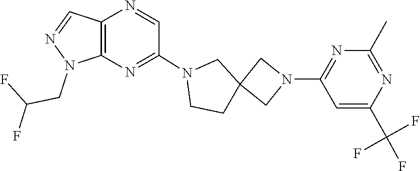 [SMILES: Cc1nc(N2CC3(CCN(c4cnc5cnn(CC(F)F)c5n4)C3)C2)cc(C(F)(F)F)n1]

Followed the General procedure A to afford the desired product as a colorless oil (49.7 mg, 455.49%). ¹H NMR (500 MHz, CDCl₃) δ 8.06 (s, 1H), 7.97 (s, 1H), 6.35 (s, 1H), 6.22 (tt, J=55.6, 4.5 Hz, 1H), 4.67 (td, J=13.3, 4.5 Hz, 2H), 4.19 (s, 4H), 3.90 (s, 2H), 3.75 (t, J=6.9 Hz, 2H), 2.58 (s, 3H), 2.40 (t, J=6.9 Hz, 2H). MS m/z: 72 [M+H]⁺.

### Example 423: 8-[1-(2,2-difluoroethyl)-1H-pyrazolo[3,4-b]pyrazin-6-yl]-2-[(2-methyl-1,3-thiazol-5-yl)methyl]-2,8-diazaspiro[4.5]decan-3-one

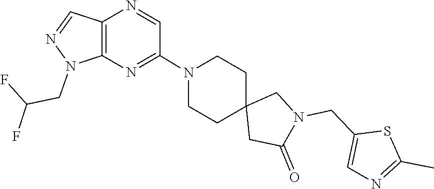 [SMILES: Cc1ncc(CN2CC3(CCN(c4cnc5cnn(CC(F)F)c5n4)CC3)CC2=O)s1]

Followed the General procedure A to afford the desired product as a brown oil (8.8 mg, 448.57%). ¹H NMR (500 MHz, CDCl₃) δ 8.22 (s, 1H), 8.04 (s, 1H), 7.48 (s, 1H), 6.20 (tt, J=55.6, 4.5 Hz, 1H), 4.69-4.59 (m, 4H), 3.86 (ddd, J=13.9, 6.5, 4.2 Hz, 2H), 3.60 (ddd, J=13.8, 8.2, 3.9 Hz, 2H), 3.19 (s, 2H), 2.69 (s, 3H), 2.42 (s, 2H), 1.77-1.68 (m, 4H). MS m/z: 13 [M+H]⁺.

### Example 424: 6-[1-(2,2,2-trifluoroethyl)-1H-pyrazolo[3,4-d]pyrimidin-6-yl]-2-[2-(trifluoromethyl)pyridin-4-yl]-2,6-diazaspiro[3.4]octane

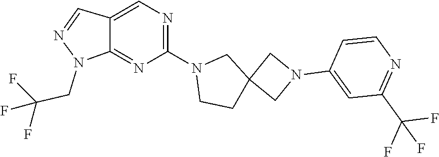

Followed the General procedure A to afford the desired product as a colorless oil (11 mg, 458.5%). ¹H NMR (500 MHz, CDCl₃) δ 8.79 (s, 1H), 8.32 (d, J=5.7 Hz, 1H), 7.94 (s, 1H), 6.63 (d, J=2.3 Hz, 1H), 6.38 (dd, J=5.7, 2.3 Hz, 1H), 4.87 (q, J=8.4 Hz, 2H), 4.12-3.98 (m, 4H), 3.86 (d, J=67.9 Hz, 4H), 2.34 (t, J=6.9 Hz, 2H). MS m/z: 24 [M+H]⁺.

### Example 425: 6-[6-(1,3,4-thiadiazol-2-yl)pyrazin-2-yl]-2-[2-(trifluoromethyl)pyridin-4-yl]-2,6-diazaspiro[3.4]octane

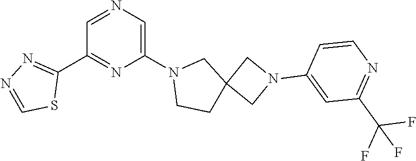 [SMILES: FC(F)(F)c1cc(N2CC3(CCN(c4cncc(-c5nncs5)n4)C3)C2)ccn1]

Followed the General procedure A to afford the desired product as a yellow wax (16 mg, 420.52%). ¹H NMR (500 MHz, CDCl₃) δ 9.20 (s, 1H), 8.88 (s, 1H), 8.33 (d, J=5.6 Hz, 1H), 8.01 (s, 1H), 6.64 (d, J=2.3 Hz, 1H), 6.39 (dd, J=5.6, 2.3 Hz, 1H), 4.11-4.02 (m, 4H), 3.85 (s, 2H), 3.70 (t, J=6.9 Hz, 2H), 2.41 (t, J=6.9 Hz, 2H). MS m/z: 37 [M+H]⁺.

### Example 426: 6-[1-(2,2,2-trifluoroethyl)-1H-pyrazolo[3,4-b]pyrazin-6-yl]-2-[2-(trifluoromethyl)pyrimidin-5-yl]-2,6-diazaspiro[3.4]octane

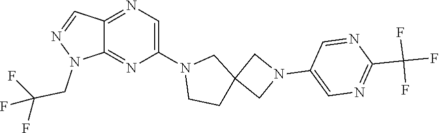 [SMILES: FC(F)(F)Cn1ncc2ncc(N3CCC4(CN(c5cnc(C(F)(F)F)nc5)C4)C3)nc21]

Followed the General procedure A to afford the desired product as a white wax (25 mg, 459.59%). ¹H NMR (500 MHz, CDCl₃) δ 8.10 (s, 1H), 8.03 (s, 2H), 7.99 (s, 1H), 4.89 (q, J=8.4 Hz, 2H), 4.18-4.10 (m, 4H), 3.93 (s, 2H), 3.76 (t, J=6.9 Hz, 2H), 2.44 (t, J=6.9 Hz, 2H). MS m/z: 60 [M+H]⁺.

### Example 427: 6-[1-(oxetan-3-yl)-1H-pyrazolo[3,4-b]pyrazin-6-yl]-2-[2-(trifluoromethyl)pyrimidin-5-yl]-2,6-diazaspiro[3.4]octane

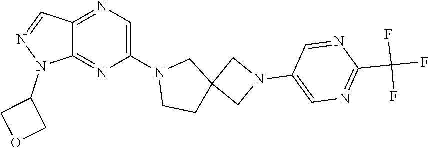 [SMILES: FC(F)(F)c1ncc(N2CC3(CCN(c4cnc5cnn(C6COC6)c5n4)C3)C2)cn1]

Followed the General procedure A to afford the desired product as a off-white solid (22.5 mg, 45%). ¹H NMR (500 MHz, DMSO-d₆) δ 8.11 (s, 1H), 8.02 (s, 2H), 7.96 (s, 1H), 5.97-5.88 (m, 1H), 5.35 (t, J=6.6 Hz, 2H), 5.05 (dd, J=7.9, 6.6 Hz, 2H), 4.18-4.08 (m, 4H), 3.92 (s, 2H), 3.76 (t, J=6.9 Hz, 2H). MS m/z: 433 [M+H]⁺.

### Example 428: 6-[3-methyl-1-(oxetan-3-yl)-1H-pyrazolo[3,4-b]pyrazin-6-yl]-2-[2-(trifluoromethyl)pyrimidin-5-yl]-2,6-diazaspiro[3.4]octane

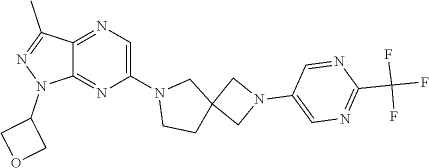 [SMILES: Cc1nn(C2COC2)c2nc(N3CCC4(CN(c5cnc(C(F)(F)F)nc5)C4)C3)cnc12]

Followed the General procedure A to afford the desired product as a white solid (25 mg, 49%). ¹H NMR (500 MHz, CDCl₃) δ 8.02 (s, 2H), 7.88 (s, 1H), 5.87 (tt, J=7.9, 6.8 Hz, 1H), 5.33 (t, J=6.6 Hz, 2H), 5.03 (dd, J=8.1, 6.7 Hz, 2H), 4.19-4.03 (m, 4H), 3.91 (s, 2H), 3.74 (t, J=6.9 Hz, 2H), 2.59 (s, 3H), 2.42 (t, J=6.9 Hz, 2H). MS m/z: 447 [M+H]⁺.

### Example 429: 6-[1-(2,2-difluoroethyl)-3-methyl-1H-pyrazolo[3,4-b]pyrazin-6-yl]-2-[2-(trifluoromethyl)pyrimidin-5-yl]-2,6-diazaspiro[3.4]octane

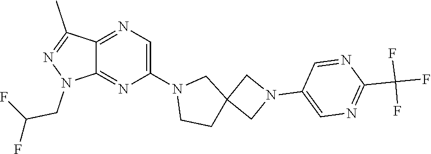 [SMILES: Cc1nn(CC(F)F)c2nc(N3CCC4(CN(c5cnc(C(F)(F)F)nc5)C4)C3)cnc12]

Followed the General procedure A to afford the desired product as a colorless oil (32 mg, 54%). ¹H NMR (500 MHz, CDCl₃) δ 8.00 (s, 2H), 7.87 (s, 1H), 6.18 (tt, J=55.7, 4.5 Hz, 2H), 4.57 (td, J=13.4, 4.5 Hz, 2H), 4.19-4.04 (m, 4H), 3.90 (s, 2H), 3.73 (t, J=6.9 Hz, 2H), 2.54 (s, 3H), 2.41 (t, J=6.9 Hz, 2H). MS m/z: 455 [M+H]⁺.

### Example 430: 6-[6-(1,3,4-thiadiazol-2-yl)pyrazin-2-yl]-2-[2-(trifluoromethyl)pyrimidin-5-yl]-2,6-diazaspiro[3.4]octane

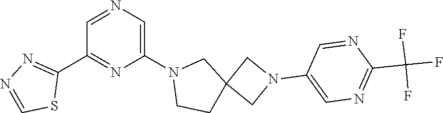 [SMILES: FC(F)(F)c1ncc(N2CC3(CCN(c4cncc(-c5nncs5)n4)C3)C2)cn1]

Followed the General procedure A to afford the desired product as a light yellow wax (22 mg, 45%). ¹H NMR (500 MHz, CDCl₃) δ 9.18 (s, 1H), 8.87 (s, 1H), 8.01 (s, 2H), 7.99 (s, 1H), 4.17-4.07 (m, 4H), 3.86 (s, 2H), 3.69 (t, J=6.9 Hz, 2H), 2.42 (t, J=6.9 Hz, 2H). MS m/z: 421 [M+H]⁺.

### Example 431: 6-[1-(2,2-difluoroethyl)-1H-pyrazolo[3,4-d]pyrimidin-6-yl]-2-[2-(trifluoromethyl)pyrimidin-5-yl]-2,6-diazaspiro[3.4]octane

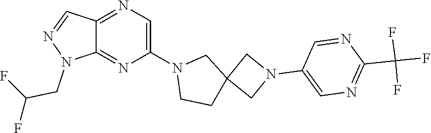 [SMILES: FC(F)Cn1ncc2ncc(N3CCC4(CN(c5cnc(C(F)(F)F)nc5)C4)C3)nc21]

Followed the General procedure A to afford the desired product as a colorless oil (7.7 mg, 15%). MS m/z: 441 [M+H]⁺.

### Example 432: 6-[1-(2,2,2-trifluoroethyl)-1H-pyrazolo[3,4-d]pyrimidin-6-yl]-2-[2-(trifluoromethyl)pyrimidin-5-yl]-2,6-diazaspiro[3.4]octane

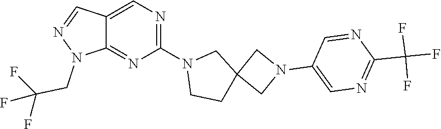 [SMILES: FC(F)(F)Cn1ncc2cnc(N3CCC4(CN(c5cnc(C(F)(F)F)nc5)C4)C3)nc21]

Followed the General procedure A to afford the desired product as a colorless oil (12 mg, 22%). ¹H NMR (500 MHz, CDCl₃) δ 8.79 (s, 1H), 8.01 (s, 2H), 7.95 (s, 1H), 4.87 (q, J=8.4 Hz, 2H), 4.19-4.05 (m, 4H), 3.95 (s, 2H), 3.79 (s, 2H), 2.37 (t, J=6.9 Hz, 2H). MS m/z: 459 [M+H]⁺.

### Example 433: 6-[1-(2,2-difluoroethyl)-3-methyl-1H-pyrazolo[3,4-d]pyrimidin-6-yl]-2-[2-(trifluoromethyl)pyrimidin-5-yl]-2,6-diazaspiro[3.4]octane

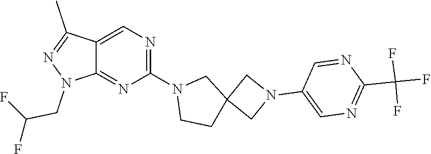 [SMILES: Cc1nn(CC(F)F)c2nc(N3CCC4(CN(c5cnc(C(F)(F)F)nc5)C4)C3)ncc12]

Followed the General procedure A to afford the desired product as a colorless oil (27 mg, 52%). ¹H NMR (500 MHz, CDCl₃) δ 8.68 (s, 1H), 8.01 (s, 2H), 6.19 (tt, J=55.7, 4.5 Hz, 1H), 4.55 (td, J=13.4, 4.5 Hz, 2H), 4.18-4.02 (m, 4H), 3.94 (s, 2H), 3.78 (s, 2H), 2.49 (s, 3H), 2.35 (t, J=6.9 Hz, 2H). MS m/z: 455 [M+H]⁺.

### Example 434: 6-[1-(oxetan-3-yl)-1H-pyrazolo[3,4-d]pyrimidin-6-yl]-2-[2-(trifluoromethyl)pyrimidin-5-yl]-2,6-diazaspiro[3.4]octane

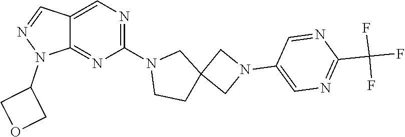 [SMILES: FC(F)(F)c1ncc(N2CC3(CCN(c4ncc5cnn(C6COC6)c5n4)C3)C2)cn1]

Followed the General procedure A to afford the desired product as a white solid (10.5 mg, 21%). ¹H NMR (500 MHz, CDCl₃) δ 8.78 (s, 1H), 8.01 (s, 2H), 7.96 (s, 1H), 5.95 (q, J=7.4 Hz, 1H), 5.31 (t, J=6.7 Hz, 2H), 5.05 (dd, J=7.9, 6.6 Hz, 2H), 4.14-4.06 (m, 4H), 3.94 (s, 2H), 3.78 (s, 2H), 2.36 (t, J=6.9 Hz, 2H). MS m/z: 433 [M+H]⁺.

### Example 435: 6-[1-(2,2-difluoroethyl)-1H-pyrazolo[3,4-b]pyrazin-6-yl]-2-[3-(difluoromethoxy)benzoyl]-2,6-diazaspiro[3.4]octane

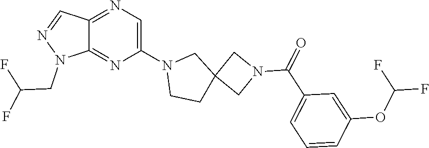 [SMILES: O=C(c1cccc(OC(F)F)c1)N1CC2(CCN(c3cnc4cnn(CC(F)F)c4n3)C2)C1]

Followed the General procedure A to afford the desired product as a colorless oil (18 mg, 64%). ¹H NMR (600 MHz, CDCl₃) δ 8.05 (s, OH), 7.95 (s, 1H), 7.50 (dt, J=7.7, 1.3 Hz, 1H), 7.45-7.40 (m, 2H), 7.24 (dd, J=8.2, 2.5 Hz, 1H), 6.55 (t, J=73.3 Hz, 1H), 6.22 (tt, J=55.7, 4.5 Hz, 1H), 4.66 (td, J=13.3, 4.5 Hz, 2H), 4.41-4.18 (m, 4H), 3.91-3.82 (m, 2H), 3.71 (dt, J=27.3, 7.3 Hz, 2H), 2.35 (q, J=7.3 Hz, 2H). MS m/z: 441 [M+H]⁺.

### Example 436: 6-[1-(2,2-difluoroethyl)-1H-pyrazolo[3,4-b]pyrazin-6-yl]-2-[6-(2,2,2-trifluoroethoxy)pyridin-3-yl]-2,6-diazaspiro[3.4]octane

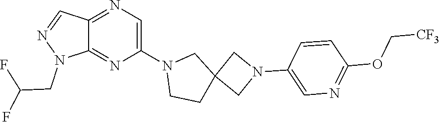 [SMILES: FC(F)Cn1ncc2ncc(N3CCC4(CN(c5ccc(OCC(F)(F)F)nc5)C4)C3)nc21]

Followed the General procedure A to afford the desired product as a colorless oil (5 mg, 12%). ¹H NMR (600 MHz, CDCl₃) δ 8.05 (s, 1H), 7.96 (s, 1H), 7.38 (d, J=2.9 Hz, 1H), 6.89 (dd, J=8.8, 3.0 Hz, 1H), 6.77 (d, J=8.8 Hz, 1H), 6.23 (tt, J=55.6, 4.5 Hz, 2H), 4.71-4.63 (m, 4H), 3.94-3.82 (m, 6H), 3.73 (t, J=6.9 Hz, 2H), 2.38 (t, J=6.9 Hz, 2H). MS m/z: 471 [M+H]⁺.

### Example 437: 6-[1-(2,2-difluoroethyl)-1H-pyrazolo[3,4-b]pyrazin-6-yl]-2-[3-methyl-5-(trifluoromethyl)pyridin-2-yl]-2,6-diazaspiro[3.4]octane

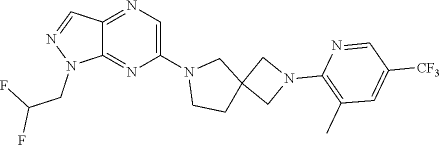 [SMILES: Cc1cc(C(F)(F)F)cnc1N1CC2(CCN(c3cnc4cnn(CC(F)F)c4n3)C2)C1]

Followed the General procedure A to afford the desired product as a white wax (14 mg, 34%). ¹H NMR (600 MHz, CDCl₃) δ 8.29 (s, 1H), 8.05 (s, 1H), 7.97 (s, 1H), 7.41 (d, J=2.3 Hz, 1H), 6.23 (tt, J=55.6, 4.5 Hz, 1H), 4.67 (td, J=13.4, 4.5 Hz, 2H), 4.29-4.17 (m, 4H), 3.88 (s, 2H), 3.74 (t, J=6.9 Hz, 2H), 2.37 (t, J=6.9 Hz, 2H), 2.26 (s, 3H). MS m/z: 455 [M+H]⁺.

### Example 438: 2-(5-chloropyrimidin-2-yl)-6-[1-(2,2-difluoroethyl)-1H-pyrazolo[3,4-b]pyrazin-6-yl]-2,6-diazaspiro[3.4]octane

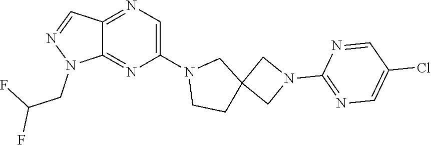 [SMILES: FC(F)Cn1ncc2ncc(N3CCC4(C3)CN(c3ncc(Cl)cn3)C4)nc21]

Followed the General procedure A to afford the desired product as a white solid (22 mg, 60%). ¹H NMR (600 MHz, CDCl₃) δ 8.27 (s, 1H), 8.05 (s, 1H), 7.97 (s, 1H), 6.23 (tt, J=55.7, 4.5 Hz, 1H), 4.66 (td, J=13.3, 4.5 Hz, 2H), 4.21-4.10 (m, 4H), 3.87 (s, 2H), 3.74 (t, J=6.9 Hz, 2H), 2.37 (t, J=6.8 Hz, 2H). MS m/z: 408 [M+H]⁺.

### Example 439: 6-[1-(2,2-difluoroethyl)-1H-pyrazolo[3,4-b]pyrazin-6-yl]-2-[2-(2,2,2-trifluoroethoxy)pyrimidin-5-yl]-2,6-diazaspiro[3.4]octane

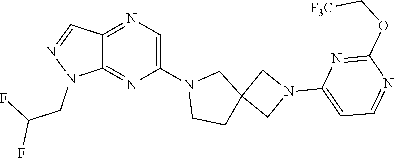 [SMILES: FC(F)Cn1ncc2ncc(N3CCC4(CN(c5ccnc(OCC(F)(F)F)n5)C4)C3)nc21]

Followed the General procedure A to afford the desired product as a white wax (24 mg, 56%). ¹H NMR (500 MHz, CDCl₃) δ 8.06 (s, 1H), 7.96 (s, 1H), 7.84 (s, 2H), 6.22 (tt, J=55.6, 4.5 Hz, 1H), 4.73 (q, J=8.4 Hz, 2H), 4.66 (td, J=13.3, 4.5 Hz, 2H), 3.96 (q, J=7.1 Hz, 4H), 3.89 (s, 2H), 3.74 (t, J=6.9 Hz, 2H), 2.40 (t, J=6.9 Hz, 2H). MS m/z: 472 [M+H]⁺.

### Example 440: 6-[1-(2,2-difluoroethyl)-1H-pyrazolo[3,4-b]pyrazin-6-yl]-2-[6-(trifluoromethyl)pyridazin-3-yl]-2,6-diazaspiro[3.4]octane

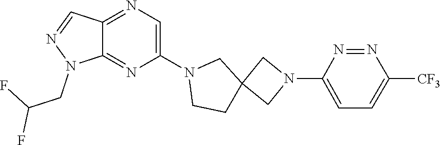 [SMILES: FC(F)Cn1ncc2ncc(N3CCC4(CN(c5ccc(C(F)(F)F)nn5)C4)C3)nc21]

Followed the General procedure A to afford the desired product as a off-white wax (12 mg, 30%). ¹H NMR (600 MHz, CDCl₃) δ 8.06 (s, 1H), 7.98 (s, 1H), 7.50 (d, J=9.2 Hz, 1H), 6.60 (d, J=9.2 Hz, 1H), 6.23 (tt, J=55.6, 4.5 Hz, 1H), 4.67 (td, J=13.3, 4.5 Hz, 2H), 4.31-4.21 (m, 4H), 3.93 (s, 2H), 3.77 (t, J=6.9 Hz, 2H), 2.43 (t, J=6.9 Hz, 2H). MS m/z: 442 [M+H]⁺.

### Example 441: 8-[1-(2,2-difluoroethyl)-1H-pyrazolo[3,4-b]pyrazin-6-yl]-2-{[4-(trifluoromethoxy)phenyl]methyl}-2,8-diazaspiro[4.5]decan-3-one

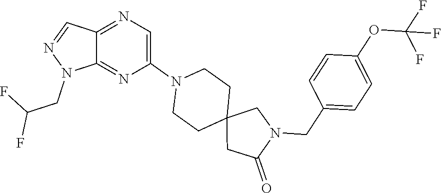 [SMILES: O=C1CC2(CCN(c3cnc4cnn(CC(F)F)c4n3)CC2)CN1Cc1ccc(OC(F)(F)F)cc1]

Followed the General procedure A to afford the desired product as a off-white wax (31 mg, 68%). ¹H NMR (600 MHz, CDCl₃) δ 8.21 (s, 1H), 8.03 (s, 1H), 7.28 (d, J=8.5 Hz, 2H), 7.20 (d, J=8.2 Hz, 2H), 6.19 (tt, J=55.6, 4.5 Hz, 1H), 4.64 (td, J=13.3, 4.5 Hz, 2H), 4.48 (s, 2H), 3.85 (ddd, J=13.8, 6.7, 3.9 Hz, 2H), 3.64-3.54 (m, 2H), 3.13 (s, 2H), 2.46 (s, 2H), 1.80-1.64 (m, 4H). MS m/z: 512 [M+H]⁺.

### Example 442: 8-[1-(2,2-difluoroethyl)-1H-pyrazolo[3,4-b]pyrazin-6-yl]-2-{[4-(difluoromethoxy)phenyl]methyl}-2,8-diazaspiro[4.5]decan-3-one

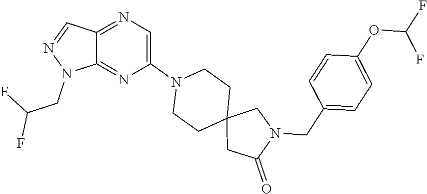 [SMILES: O=C1CC2(CCN(c3cnc4cnn(CC(F)F)c4n3)CC2)CN1Cc1ccc(OC(F)F)cc1]

Followed the General procedure A to afford the desired product as a colorless oil (30 mg, 30%). ¹H NMR (600 MHz, CDCl₃) δ 8.21 (s, 1H), 8.03 (s, 1H), 7.25 (d, J=8.6 Hz, 2H), 7.10 (d, J=8.4 Hz, 2H), 6.51 (t, J=73.7 Hz, 1H), 6.20 (tt, J=55.6, 4.5 Hz, 1H), 4.64 (td, J=13.3, 4.5 Hz, 2H), 4.46 (s, 2H), 3.84 (ddd, J=13.8, 6.6, 3.9 Hz, 2H), 3.65-3.55 (m, 2H), 3.12 (s, 2H), 2.46 (s, 2H), 1.77-1.61 (m, 4H). MS m/z: 494 [M+H]⁺.

### Example 443: 8-[1-(2,2-difluoroethyl)-1H-pyrazolo[3,4-b]pyrazin-6-yl]-2-{[4-(trifluoromethyl)phenyl]methyl}-2,8-diazaspiro[4.5]decan-3-one

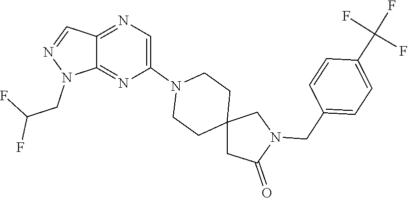 [SMILES: O=C1CC2(CCN(c3cnc4cnn(CC(F)F)c4n3)CC2)CN1Cc1ccc(C(F)(F)F)cc1]

Followed the General procedure A to afford the desired product as a white foam (28 mg, 64%). ¹H NMR (600 MHz, CDCl₃) δ 8.21 (s, 1H), 8.03 (s, OH), 7.62 (d, J=7.9 Hz, 2H), 7.37 (d, J=7.9 Hz, 2H), 6.19 (tt, J=55.6, 4.5 Hz, 1H), 4.64 (td, J=13.3, 4.5 Hz, 2H), 4.53 (s, 2H), 3.86 (ddd, J=13.7, 6.6, 3.9 Hz, 2H), 3.63-3.56 (m, 2H), 3.13 (s, 2H), 2.48 (s, 2H), 1.84-1.64 (m, 4H). MS m/z: 496 [M+H]⁺.

### Example 444: 8-[1-(2,2-difluoroethyl)-1H-pyrazolo[3,4-b]pyrazin-6-yl]-2-[(4-fluorophenyl)methyl]-2,8-diazaspiro[4.5]decan-3-one

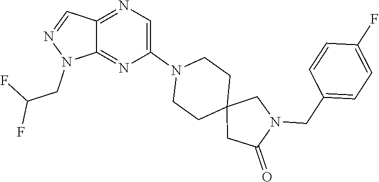 [SMILES: O=C1CC2(CCN(c3cnc4cnn(CC(F)F)c4n3)CC2)CN1Cc1ccc(F)cc1]

Followed the General procedure A to afford the desired product as a white wax (27 mg, 68%). ¹H NMR (600 MHz, CDCl₃) δ 8.21 (s, 1H), 8.03 (s, 1H), 7.25-7.20 (m, 2H), 7.07-7.01 (m, 2H), 6.20 (tt, J=55.6, 4.5 Hz, 1H), 4.64 (td, J=13.3, 4.5 Hz, 2H), 4.44 (s, 2H), 3.83 (ddd, J=13.8, 6.6, 3.9 Hz, 2H), 3.65-3.54 (m, 2H), 3.11 (s, 2H), 2.45 (s, 2H), 1.77-1.62 (m, 4H). MS m/z: 446 [M+H]⁺.

### Example 445: 2-[(4-chlorophenyl)methyl]-8-[1-(2,2-difluoroethyl)-1H-pyrazolo[3,4-b]pyrazin-6-yl]-2,8-diazaspiro[4.5]decan-3-one

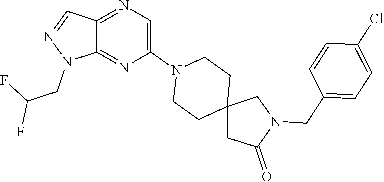 [SMILES: O=C1CC2(CCN(c3cnc4cnn(CC(F)F)c4n3)CC2)CN1Cc1ccc(Cl)cc1]

Followed the General procedure A to afford the desired product as a white wax (20 mg, 49%). ¹H NMR (600 MHz, CDCl₃) δ 8.21 (s, 1H), 8.03 (s, 1H), 7.32 (d, J=8.4 Hz, 2H), 7.19 (d, J=8.4 Hz, 2H), 6.20 (tt, J=55.6, 4.5 Hz, 1H), 4.64 (td, J=13.3, 4.5 Hz, 2H), 4.44 (s, 2H), 3.84 (ddd, J=13.8, 6.7, 3.9 Hz, 2H), 3.60 (ddd, J=13.8, 8.6, 3.6 Hz, 2H), 3.11 (s, 2H), 2.45 (s, 2H), 1.79-1.61 (m, 4H). MS m/z: 462 [M+H]⁺.

### Example 446: 8-[1-(2,2-difluoroethyl)-1H-pyrazolo[3,4-b]pyrazin-6-yl]-2-{[2-fluoro-4-(trifluoromethyl)phenyl]methyl}-2,8-diazaspiro[4.5]decan-3-one

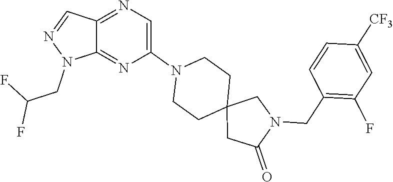 [SMILES: O=C1CC2(CCN(c3cnc4cnn(CC(F)F)c4n3)CC2)CN1Cc1ccc(C(F)(F)F)cc1F]

Followed the General procedure A to afford the desired product as a off-white wax (30 mg, 66%). ¹H NMR (600 MHz, CDCl₃) δ 8.22 (s, 1H), 8.04 (s, 1H), 7.52-7.39 (m, 2H), 7.39-7.31 (m, 1H), 6.20 (tt, J=55.6, 4.5 Hz, 1H), 4.64 (td, J=13.3, 4.5 Hz, 2H), 4.59 (s, 2H), 3.88 (ddd, J=13.8, 6.5, 4.0 Hz, 2H), 3.60 (ddd, J=13.2, 8.5, 3.8 Hz, 2H), 3.21 (s, 2H), 2.45 (s, 2H), 1.81-1.65 (m, 4H). MS m/z: 514 [M+H]⁺.

### Example 447: 8-[1-(2,2-difluoroethyl)-1H-pyrazolo[3,4-b]pyrazin-6-yl]-2-[(2,5-difluorophenyl)methyl]-2,8-diazaspiro[4.5]decan-3-one

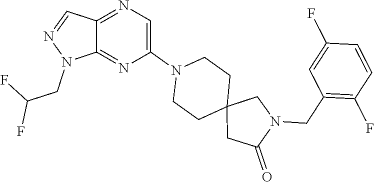 [SMILES: O=C1CC2(CCN(c3cnc4cnn(CC(F)F)c4n3)CC2)CN1Cc1cc(F)ccc1F]

Followed the General procedure A to afford the desired product as a white wax (32 mg, 78%). ¹H NMR (600 MHz, CDCl₃) δ 8.22 (s, 1H), 8.03 (s, 1H), 7.09-6.94 (m, 3H), 6.20 (tt, J=55.6, 4.5 Hz, 1H), 4.64 (td, J=13.4, 4.5 Hz, 2H), 4.53-4.50 (m, 2H), 3.87 (ddd, J=13.8, 6.5, 4.0 Hz, 2H), 3.60 (ddd, J=13.8, 8.5, 3.7 Hz, 2H), 3.19 (s, 2H), 2.45 (s, 2H), 1.79-1.67 (m, 4H). MS m/z: 464 [M+H]⁺.

### Example 448: 2-[(4-chloro-3-fluorophenyl)methyl]-8-[1-(2,2-difluoroethyl)-1H-pyrazolo[3,4-b]pyrazin-6-yl]-2,8-diazaspiro[4.5]decan-3-one

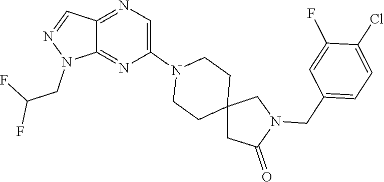 [SMILES: O=C1CC2(CCN(c3cnc4cnn(CC(F)F)c4n3)CC2)CN1Cc1ccc(Cl)c(F)c1]

Followed the General procedure A to afford the desired product as a colorless oil (34 mg, 80%). ¹H NMR (600 MHz, CDCl₃) δ 8.22 (s, 1H), 8.03 (s, 1H), 7.38 (t, J=7.8 Hz, 1H), 7.05 (dd, J=9.5, 2.0 Hz, 1H), 6.99 (dd, J=8.1, 2.0 Hz, 1H), 6.20 (tt, J=55.6, 4.5 Hz, 1H), 4.64 (td, J=13.4, 4.5 Hz, 2H), 4.44 (s, 2H), 3.86 (ddd, J=13.8, 6.6, 3.9 Hz, 2H), 3.64-3.54 (m, 2H), 3.13 (s, 2H), 2.46 (s, 2H), 1.80-1.64 (m, 4H). MS m/z: 480 [M+H]⁺.

### Example 449: 2-[(3-chloro-4-fluorophenyl)methyl]-8-[1-(2,2-difluoroethyl)-1H-pyrazolo[3,4-b]pyrazin-6-yl]-2,8-diazaspiro[4.5]decan-3-one

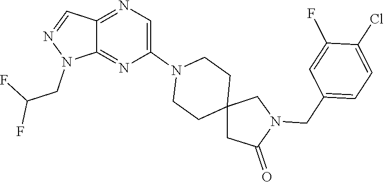 [SMILES: O=C1CC2(CCN(c3cnc4cnn(CC(F)F)c4n3)CC2)CN1Cc1ccc(Cl)c(F)c1]

Followed the General procedure A to afford the desired product as a white foam (36 mg, 84%). ¹H NMR (600 MHz, CDCl₃) δ 8.22 (s, 1H), 8.03 (s, 1H), 7.31 (dd, J=7.1, 1.8 Hz, 1H), 7.15-7.09 (m, 2H), 6.20 (tt, J=55.6, 4.5 Hz, 1H), 4.64 (td, J=13.3, 4.5 Hz, 2H), 4.42 (s, 2H), 3.86 (ddd, J=13.7, 6.5, 3.9 Hz, 2H), 3.60 (ddd, J=13.9, 8.6, 3.6 Hz, 2H), 3.13 (s, 2H), 2.46 (s, 2H), 1.79-1.62 (m, 4H). MS m/z: 480 [M+H]⁺.

### Example 450: 6-[1-(2,2-difluoroethyl)-1H-pyrazolo[3,4-b]pyrazin-6-yl]-2-[6-(difluoromethoxy)pyridin-3-yl]-2,6-diazaspiro[3.4]octane

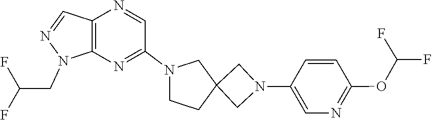 [SMILES: FC(F)Cn1ncc2ncc(N3CCC4(CN(c5ccc(OC(F)F)nc5)C4)C3)nc21]

Followed the General procedure A to afford the desired product as a colorless oil (12 mg, 18%). ¹H NMR (600 MHz, CDCl₃) δ 8.05 (s, 1H), 7.96 (s, 1H), 7.43 (d, J=3.0 Hz, 1H), 7.29 (t, J=73.8 Hz, 1H), 6.89 (dd, J=8.7, 3.0 Hz, 1H), 6.81 (d, J=8.7 Hz, 1H), 6.22 (tt, J=55.6, 4.5 Hz, 1H), 4.66 (td, J=13.3, 4.6 Hz, 2H), 3.96-3.85 (m, 6H), 3.73 (t, J=6.9 Hz, 2H), 2.38 (t, J=6.9 Hz, 2H). MS m/z: 439 [M+H]⁺.

### Example 451: 6-[1-(2,2-difluoroethyl)-1H-pyrazolo[3,4-b]pyrazin-6-yl]-2-[6-(trifluoromethoxy)pyridin-3-yl]-2,6-diazaspiro[3.4]octane

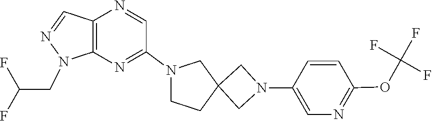 [SMILES: FC(F)Cn1ncc2ncc(N3CCC4(CN(c5ccc(OC(F)(F)F)nc5)C4)C3)nc21]

Followed the General procedure A to afford the desired product as a white wax (4.5 mg, 10%). ¹H NMR (500 MHz, CDCl₃) δ 8.06 (s, 1H), 7.96 (s, 1H), 7.55 (dd, J=3.1, 0.6 Hz, 1H), 6.94 (d, J=8.7 Hz, 1H), 6.87 (dd, J=8.7, 3.0 Hz, 1H), 6.35-6.09 (m, 1H), 4.66 (td, J=13.3, 4.5 Hz, 2H), 4.03-3.92 (m, 4H), 3.88 (s, 2H), 3.74 (t, J=6.9 Hz, 2H), 2.39 (t, J=6.9 Hz, 2H). MS m/z: 457 [M+H]⁺.

### Example 452: 6-[1-(2,2-difluoroethyl)-1H-pyrazolo[3,4-b]pyrazin-6-yl]-2-[2-(difluoromethoxy)pyridin-4-yl]-2,6-diazaspiro[3.4]octane

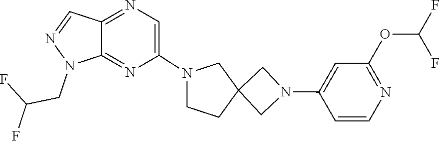 [SMILES: FC(F)Cn1ncc2ncc(N3CCC4(CN(c5ccnc(OC(F)F)c5)C4)C3)nc21]

Followed the General procedure A to afford the desired product as a yellow oil (36 mg, 43%). ¹H NMR (600 MHz, CDCl₃) δ 8.05 (s, 1H), 7.96 (s, 1H), 7.85 (d, J=5.8 Hz, 1H), 7.43 (t, J=73.6 Hz, 1H), 6.22 (tt, J=55.6, 4.5 Hz, 2H), 6.13 (dd, J=5.9, 2.1 Hz, 1H), 5.81 (d, J=2.1 Hz, 1H), 4.66 (td, J=13.3, 4.5 Hz, 2H), 4.01 (d, J=7.7 Hz, 2H), 3.97 (d, J=7.8 Hz, 2H), 3.88 (s, 2H), 3.74 (t, J=6.8 Hz, 2H), 2.38 (t, J=6.8 Hz, 2H). MS m/z: 438 [M+H]⁺.

### Example 453: 6-[1-(2,2-difluoroethyl)-1H-pyrazolo[3,4-b]pyrazin-6-yl]-2-[5-fluoro-4-(trifluoromethyl)pyridin-2-yl]-2,6-diazaspiro[3.4]octane

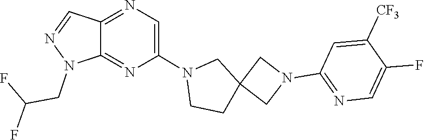 [SMILES: Fc1cnc(N2CC3(CCN(c4cnc5cnn(CC(F)F)c5n4)C3)C2)cc1C(F)(F)F]

Followed the General procedure A to afford the desired product as a 10 (10 mg, 24%). ¹H NMR (600 MHz, CDCl₃) δ 8.17 (d, J=2.1 Hz, 1H), 8.05 (s, 1H), 7.97 (s, 1H), 6.44 (d, J=4.3 Hz, 1H), 6.23 (tt, J=55.6, 4.5 Hz, 1H), 4.66 (td, J=13.3, 4.5 Hz, 2H), 4.13-4.01 (m, 4H), 3.88 (s, 2H), 3.74 (t, J=6.9 Hz, 2H), 2.38 (t, J=6.9 Hz, 2H). MS m/z: 458 [M+H]⁺.

### Example 454: 2-[6-methyl-2-(trifluoromethyl)pyrimidin-4-yl]-6-[1-(oxetan-3-yl)-1H-pyrazolo[3,4-b]pyrazin-6-yl]-2,6-diazaspiro[3.4]octane

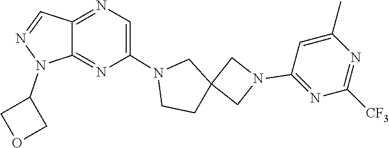 [SMILES: Cc1cc(N2CC3(CCN(c4cnc5cnn(C6COC6)c5n4)C3)C2)nc(C(F)(F)F)n1]

Followed the General procedure A to afford the desired product as a light yellow oil (14 mg, 40%). ¹H NMR (600 MHz, CDCl₃) δ 8.10 (s, 1H), 7.95 (s, 1H), 6.15 (s, 1H), 5.98-5.90 (m, 1H), 5.35 (t, J=6.6 Hz, 2H), 5.05 (dd, J=7.9, 6.6 Hz, 2H), 4.16 (d, J=22.5 Hz, 4H), 3.88 (s, 2H), 3.74 (t, J=6.9 Hz, 2H), 2.44 (s, 3H), 2.39 (t, J=6.9 Hz, 2H). MS m/z: 447 [M+H]⁺.

### Example 455: 6-[3-methyl-1-(oxetan-3-yl)-1H-pyrazolo[3,4-b]pyrazin-6-yl]-2-[2-methyl-6-(trifluoromethyl)pyrimidin-4-yl]-2,6-diazaspiro[3.4]octane

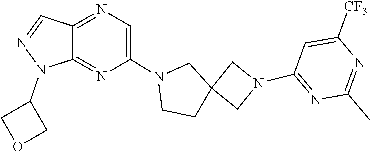 [SMILES: Cc1nc(N2CC3(CCN(c4cnc5cnn(C6COC6)c5n4)C3)C2)cc(C(F)(F)F)n1]

Followed the General procedure A to afford the desired product as a white wax (19 mg, 53%). ¹H NMR (600 MHz, CDCl₃) δ 7.88 (s, 1H), 6.34 (s, 1H), 5.91-5.83 (m, 1H), 5.33 (t, J=6.6 Hz, 2H), 5.03 (dd, J=7.9, 6.5 Hz, 2H), 4.15 (dd, J=22.4, 14.4 Hz, 4H), 3.88 (s, 2H), 3.73 (t, J=6.9 Hz, 2H), 2.59 (s, 3H), 2.57 (s, 3H), 2.38 (t, J=6.9 Hz, 2H). MS m/z: 461 [M+H]⁺.

### Example 456: 2-[6-methyl-2-(trifluoromethyl)pyrimidin-4-yl]-8-[1-(oxetan-3-yl)-1H-pyrazolo[3,4-b]pyrazin-6-yl]-2,8-diazaspiro[4.5]decan-3-one

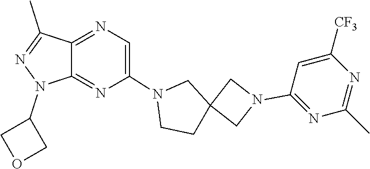 [SMILES: Cc1nc(N2CC3(CCN(c4cnc5c(C)nn(C6COC6)c5n4)C3)C2)cc(C(F)(F)F)n1]

Followed the General procedure A to afford the desired product as a white wax (19 mg, 56%). ¹H NMR (600 MHz, CDCl₃) δ 8.41 (s, 1H), 8.25 (s, 1H), 8.10 (s, 1H), 5.97-5.89 (m, 1H), 5.34 (t, J=6.6 Hz, 2H), 5.06 (dd, J=8.0, 6.7 Hz, 2H), 4.02 (s, 2H), 3.95 (ddd, J=13.7, 6.9, 4.0 Hz, 2H), 3.71 (ddd, J=13.7, 8.0, 3.8 Hz, 2H), 2.70 (s, 2H), 2.63 (s, 3H), 1.86 (m, 4H). MS m/z: 489 [M+H]⁺.

### Example 457: 2-[2-methyl-6-(trifluoromethyl)pyrimidin-4-yl]-8-[1-(oxetan-3-yl)-1H-pyrazolo[3,4-b]pyrazin-6-yl]-2,8-diazaspiro[4.5]decan-3-one

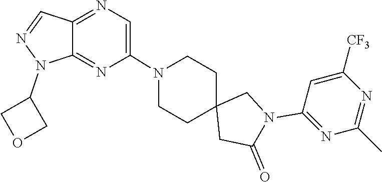 [SMILES: Cc1nc(N2CC3(CCN(c4cnc5cnn(C6COC6)c5n4)CC3)CC2=O)cc(C(F)(F)F)n1]

Followed the General procedure A to afford the desired product as a colorless oil (18 mg, 53%). ¹H NMR (600 MHz, CDCl₃) δ 8.57 (s, 1H), 8.26 (s, 1H), 8.10 (s, 1H), 5.99-5.87 (m, 1H), 5.34 (t, J=6.6 Hz, 2H), 5.06 (dd, J=7.9, 6.6 Hz, 2H), 4.01 (s, 2H), 3.94 (ddd, J=13.8, 6.8, 4.3 Hz, 2H), 3.73 (ddd, J=13.7, 7.6, 4.1 Hz, 2H), 2.72 (s, 3H), 2.71 (s, 2H), 1.93-1.79 (m, 4H). MS m/z: 489 [M+H]⁺.

### Example 458: 8-[3-methyl-1-(oxetan-3-yl)-1H-pyrazolo[3,4-b]pyrazin-6-yl]-2-[6-methyl-2-(trifluoromethyl)pyrimidin-4-yl]-2,8-diazaspiro[4.5]decan-3-one

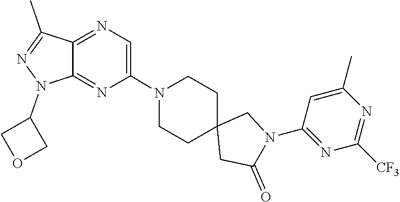 [SMILES: Cc1cc(N2CC3(CCN(c4cnc5c(C)nn(C6COC6)c5n4)CC3)CC2=O)nc(C(F)(F)F)n1]

Followed the General procedure A to afford the desired product as a colorless oil (15 mg, 43%). ¹H NMR (600 MHz, CDCl₃) δ 8.41 (s, 1H), 8.17 (s, 1H), 5.87 (tt, J=8.0, 6.7 Hz, 1H), 5.32 (t, J=6.7 Hz, 2H), 5.03 (dd, J=7.9, 6.5 Hz, 2H), 4.01 (s, 2H), 3.93 (ddd, J=13.9, 6.9, 3.9 Hz, 2H), 3.69 (ddd, J=13.7, 8.0, 3.8 Hz, 2H), 2.70 (s, 2H), 2.63 (s, 3H), 2.59 (s, 3H), 1.93-1.79 (m, 4H). MS m/z: 503 [M+H]⁺.

### Example 459: 8-[3-methyl-1-(oxetan-3-yl)-1H-pyrazolo[3,4-b]pyrazin-6-yl]-2-[2-methyl-6-(trifluoromethyl)pyrimidin-4-yl]-2,8-diazaspiro[4.5]decan-3-one

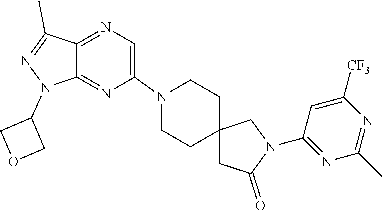 [SMILES: Cc1nc(N2CC3(CCN(c4cnc5c(C)nn(C6COC6)c5n4)CC3)CC2=O)cc(C(F)(F)F)n1]

Followed the General procedure A to afford the desired product as a colorless oil (14 mg, 40%). ¹H NMR (500 MHz, CDCl₃) δ 8.57 (s, 1H), 8.18 (s, 1H), 5.87 (p, J=7.3 Hz, 1H), 5.32 (t, J=6.6 Hz, 2H), 5.04 (t, J=7.2 Hz, 2H), 4.01 (s, 2H), 3.92 (ddd, J=13.9, 6.7, 4.3 Hz, 2H), 3.71 (ddd, J=13.7, 7.4, 4.2 Hz, 2H), 2.72 (s, 3H), 2.70 (s, 2H), 2.59 (s, 3H), 1.95-1.76 (m, 4H). MS m/z: 503 [M+H]⁺.

### Example 460: 6-[3-methyl-1-(oxetan-3-yl)-1H-pyrazolo[3,4-d]pyrimidin-6-yl]-2-[2-(trifluoromethyl)pyrimidin-5-yl]-2,6-diazaspiro[3.4]octane

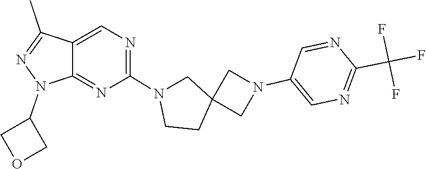 [SMILES: Cc1nn(C2COC2)c2nc(N3CCC4(CN(c5cnc(C(F)(F)F)nc5)C4)C3)ncc12]

Followed the General procedure A to afford the desired product as a white wax (28.9 mg, 56%). ¹H NMR (600 MHz, CDCl₃) δ 8.67 (s, 1H), 8.00 (s, 2H), 5.90-5.83 (m, 1H), 5.29 (t, J=6.6 Hz, 2H), 5.02 (dd, J=7.8, 6.5 Hz, 2H), 4.15-4.05 (m, 4H), 3.93 (s, 2H), 3.77 (t, J=7.0 Hz, 2H), 2.53 (s, 3H), 2.35 (t, J=6.9 Hz, 2H). MS m/z: 447 [M+H]⁺.

### Example 461: 6-[1-(2,2-difluoroethyl)-1H-pyrazolo[3,4-b]pyrazin-6-yl]-2-(2-methylpyrimidin-5-yl)-2,6-diazaspiro[3.4]octane

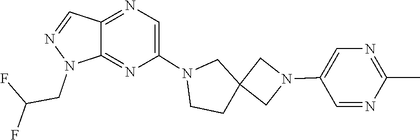 [SMILES: Cc1ncc(N2CC3(CCN(c4cnc5cnn(CC(F)F)c5n4)C3)C2)cn1]

Followed the General procedure A to afford the desired product as a off-white wax (13.2 mg, 25%). ¹H NMR (600 MHz, CDCl₃) δ 8.05 (s, 1H), 7.97 (s, 1H), 7.94 (s, 2H), 6.22 (tt, J=55.6, 4.5 Hz, 1H), 4.66 (td, J=13.3, 4.5 Hz, 2H), 4.06-3.97 (m, 4H), 3.89 (s, 2H), 3.74 (t, J=6.9 Hz, 2H), 2.64 (s, 3H), 2.40 (t, J=6.9 Hz, 2H). MS m/z: 387 [M+H]⁺.

### Example 462: 6-[1-(2,2-difluoroethyl)-1H-pyrazolo[3,4-b]pyrazin-6-yl]-2-(2-ethylpyrimidin-5-yl)-2,6-diazaspiro[3.4]octane

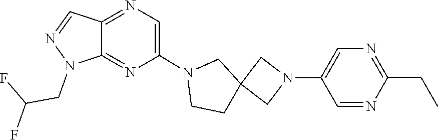 [SMILES: CCc1ncc(N2CC3(CCN(c4cnc5cnn(CC(F)F)c5n4)C3)C2)cn1]

Followed the General procedure A to afford the desired product as a off-white wax (12.6 mg, 23%). ¹H NMR (600 MHz, CDCl₃) δ 8.05 (s, 1H), 7.97 (s, 3H), 6.22 (tt, J=55.7, 4.5 Hz, 1H), 4.66 (td, J=13.4, 4.5 Hz, 2H), 4.06-3.94 (m, 4H), 3.89 (s, 2H), 3.74 (t, J=6.9 Hz, 2H), 2.90 (q, J=7.6 Hz, 2H), 2.40 (t, J=6.9 Hz, 2H), 1.33 (t, J=7.6 Hz, 3H). MS m/z: 401 [M+H]⁺.

### Example 463: 2-[6-methyl-2-(trifluoromethyl)pyrimidin-4-yl]-6-[1-(2,2,2-trifluoroethyl)-1H-pyrazolo[3,4-b]pyrazin-6-yl]-2,6-diazaspiro[3.4]octane

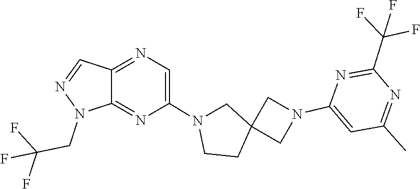 [SMILES: Cc1cc(N2CC3(CCN(c4cnc5cnn(CC(F)(F)F)c5n4)C3)C2)nc(C(F)(F)F)n1]

Followed the General procedure A to afford the desired product as a colorless oil (15.2 mg, 29%). ¹H NMR (600 MHz, CDCl₃) δ 8.10 (s, 1H), 7.98 (s, 1H), 6.15 (s, 1H), 4.89 (q, J=8.4 Hz, 2H), 4.16 (d, J=24.6 Hz, 4H), 3.89 (s, 2H), 3.75 (t, J=6.9 Hz, 2H), 2.44 (s, 3H), 2.39 (t, J=6.9 Hz, 2H). MS m/z: 473 [M+H]⁺.

### Example 464: 6-[1-(2,2-difluoroethyl)-3-methyl-1H-pyrazolo[3,4-b]pyrazin-6-yl]-2-[6-methyl-2-(trifluoromethyl)pyrimidin-4-yl]-2,6-diazaspiro[3.4]octane

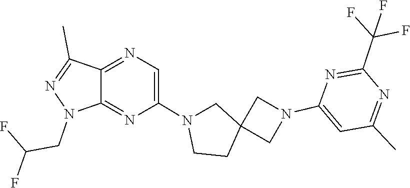

Followed the General procedure A to afford the desired product as a white solid (13.3 mg, 26%). ¹H NMR (600 MHz, CDCl₃) δ 7.89 (s, 1H), 6.22 (tt, J=55.7, 4.5 Hz, 1H), 6.17 (s, 1H), 4.59 (td, J=13.4, 4.5 Hz, 2H), 4.16 (d, J=26.9 Hz, 4H), 3.88 (s, 2H), 3.74 (t, J=6.9 Hz, 2H), 2.55 (s, 3H), 2.44 (s, 3H), 2.38 (t, J=6.9 Hz, 2H). MS m/z: 469 [M+H]⁺.

### Example 465: 2-[6-methyl-2-(trifluoromethyl)pyrimidin-4-yl]-6-[6-(1,3,4-thiadiazol-2-yl)pyrazin-2-yl]-2,6-diazaspiro[3.4]octane

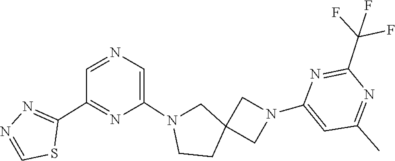 [SMILES: Cc1cc(N2CC3(CCN(c4cncc(-c5nncs5)n4)C3)C2)nc(C(F)(F)F)n1]

Followed the General procedure A to afford the desired product as a yellow wax (4.8 mg, 10%). ¹H NMR (600 MHz, CDCl₃) δ 9.19 (s, 1H), 8.88 (s, 1H), 8.01 (s, 1H), 6.15 (s, 1H), 4.17 (d, J=26.1 Hz, 4H), 3.84 (s, 2H), 3.69 (t, J=6.9 Hz, 2H), 2.44 (s, 3H), 2.39 (t, J=6.9 Hz, 2H). MS m/z: 435 [M+H]⁺.

### Example 466: 6-[1-(2,2-difluoroethyl)-1H-pyrazolo[3,4-d]pyrimidin-6-yl]-2-[6-methyl-2-(trifluoromethyl)pyrimidin-4-yl]-2,6-diazaspiro[3.4]octane

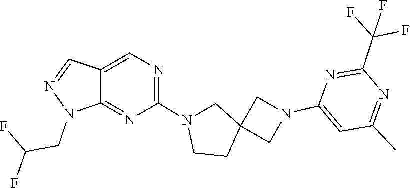

Followed the General procedure A to afford the desired product as a yellow wax (7 mg, 11%). ¹H NMR (500 MHz, CDCl₃) δ 8.78 (s, 1H), 7.90 (s, 1H), 6.36-6.10 (m, 2H), 4.64 (td, J=13.3, 4.5 Hz, 2H), 4.14 (s, 4H), 3.91 (s, 2H), 3.78 (s, 2H), 2.46-2.39 (m, 3H), 2.32 (t, J=6.9 Hz, 2H). MS m/z: 455 [M+H]⁺.

### Example 467: 2-[6-methyl-2-(trifluoromethyl)pyrimidin-4-yl]-6-[1-(2,2,2-trifluoroethyl)-1H-pyrazolo[3,4-d]pyrimidin-6-yl]-2,6-diazaspiro[3.4]octane

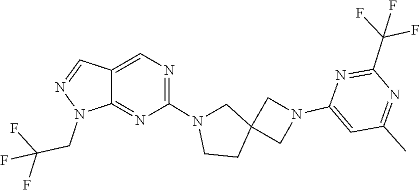 [SMILES: Cc1cc(N2CC3(CCN(c4ncc5cnn(CC(F)(F)F)c5n4)C3)C2)nc(C(F)(F)F)n1]

Followed the General procedure A to afford the desired product as a colorless oil (6.5 mg, 12%). ¹H NMR (600 MHz, CDCl₃) δ 8.79 (s, 1H), 7.94 (s, 1H), 6.13 (s, 1H), 4.86 (q, J=8.4 Hz, 2H), 4.14 (m, 4H), 3.92 (s, 2H), 3.78 (s, 2H), 2.43 (s, 3H), 2.32 (t, J=6.9 Hz, 2H). MS m/z: 473 [M+H]⁺.

### Example 468: 6-[1-(2,2-difluoroethyl)-3-methyl-1H-pyrazolo[3,4-d]pyrimidin-6-yl]-2-[6-methyl-2-(trifluoromethyl)pyrimidin-4-yl]-2,6-diazaspiro[3.4]octane

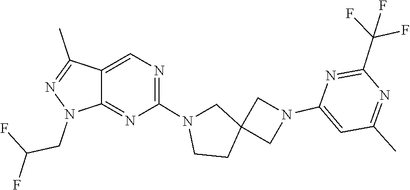 [SMILES: Cc1cc(N2CC3(CCN(c4ncc5c(C)nn(CC(F)F)c5n4)C3)C2)nc(C(F)(F)F)n1]

Followed the General procedure A to afford the desired product as a colorless oil (14.8 mg, 29%). ¹H NMR (600 MHz, CDCl₃) δ 8.68 (s, 1H), 6.22 (tt, J=55.7, 4.5 Hz, 1H), 6.13 (s, 1H), 4.55 (td, J=13.4, 4.5 Hz, 3H), 4.13 (d, J=28.1 Hz, 4H), 3.91 (s, 2H), 3.77 (s, 2H), 2.49 (s, 3H), 2.43 (s, 3H), 2.31 (t, J=6.9 Hz, 2H). MS m/z: 469 [M+H]⁺.

### Example 469: 6-[1-(oxan-2-yl)-1H-pyrazolo[3,4-d]pyrimidin-6-yl]-2-[2-(trifluoromethyl)pyrimidin-5-yl]-2,6-diazaspiro[3.4]octane

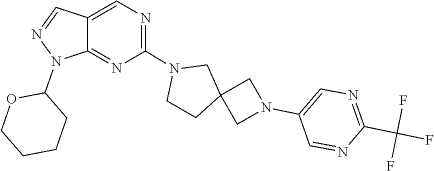 [SMILES: FC(F)(F)c1ncc(N2CC3(CCN(c4ncc5cnn(C6CCCCO6)c5n4)C3)C2)cn1]

Followed the General procedure A to afford the desired product as a off-white solid (6.5 mg, 42%). ¹H NMR (500 MHz, DMSO) δ 8.82 (dd, J=3.5, 1.8 Hz, 1H), 8.20 (d, J=12.8 Hz, 2H), 7.87 (d, J=3.2 Hz, 1H), 5.42 (ddd, J=10.6, 4.8, 2.1 Hz, 1H), 4.22-4.00 (m, 7H), 3.87 (td, J=10.9, 5.7 Hz, 2H), 3.60-3.50 (m, 1H), 2.44 (t, J=7.3 Hz, 1H), 2.31 (t, J=7.0 Hz, 1H), 2.16-2.06 (m, 1H), 1.91 (dd, J=7.2, 3.4 Hz, 1H), 1.62 (dt, J=8.7, 2.8 Hz, 2H), 1.47 (s, 2H). MS m/z: 461 [M+H]⁺.

### Example 470: 6-[1-(oxan-2-yl)-1H-pyrazolo[3,4-b]pyrazin-6-yl]-2-[2-(trifluoromethyl)pyrimidin-5-yl]-2,6-diazaspiro[3.4]octane

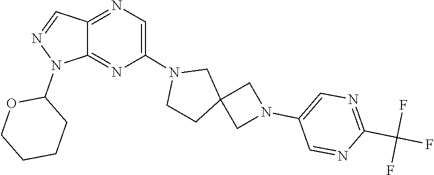 [SMILES: FC(F)(F)c1ncc(N2CC3(CCN(c4cnc5cnn(C6CCCCO6)c5n4)C3)C2)cn1]

Followed the General procedure A to afford the desired product as a yellow wax (6.5 mg, 47%). ¹H NMR (500 MHz, DMSO) δ 8.19 (s, 2H), 8.09 (d, J=3.1 Hz, 2H), 5.74 (dd, J=10.4, 2.5 Hz, 1H), 4.15-4.06 (m, 4H), 3.93 (d, J=11.6 Hz, 1H), 3.86 (s, 2H), 3.69-3.58 (m, 3H), 2.88 (s, 1H), 2.72 (d, J=0.6 Hz, 1H), 2.32 (s, 1H), 2.01 (d, J=13.4 Hz, 1H), 1.83 (dd, J=12.9, 3.1 Hz, 1H), 1.77-1.66 (m, 1H), 1.57-1.51 (m, 2H). MS m/z: 461 [M+H]⁺.

### Example 471: 6-[3-methyl-1-(oxetan-3-yl)-1H-pyrazolo[3,4-d]pyrimidin-6-yl]-2-[6-methyl-2-(trifluoromethyl)pyrimidin-4-yl]-2,6-diazaspiro[3.4]octane

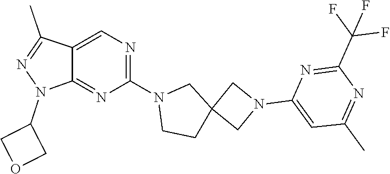

Followed the General procedure A to afford the desired product as a colorless oil (31 mg, 36%). ¹H NMR (500 MHz, CDCl₃) δ 8.66 (s, 1H), 6.13 (s, 1H), 5.86 (p, J=7.3 Hz, 1H), 5.29 (t, J=6.5 Hz, 2H), 5.02 (t, J=7.2 Hz, 2H), 4.13 (s, 4H), 3.90 (s, 2H), 3.76 (s, 2H), 2.53 (s, 3H), 2.43 (s, 3H), 2.30 (t, J=6.9 Hz, 2H). MS m/z: 461 [M+H]⁺.

### Example 472: 2-[1-(2,2-difluoroethyl)-1H-pyrazolo[3,4-b]pyrazin-6-yl]-7-[2-(trifluoromethyl)pyridin-4-yl]-2,7-diazaspiro[4.4]nonane

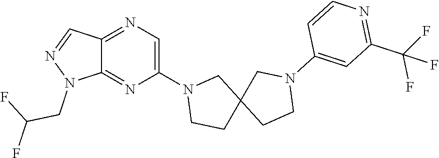 [SMILES: FC(F)Cn1ncc2ncc(N3CCC4(CCN(c5ccnc(C(F)(F)F)c5)C4)C3)nc21]

Followed the General procedure A to afford the desired product as a white wax (20 mg, 81%). ¹H NMR (600 MHz, CDCl₃) δ 8.32 (d, J=5.8 Hz, 1H), 8.06 (s, 1H), 7.97 (s, 1H), 6.76 (d, J=2.4 Hz, 1H), 6.50 (dd, J=5.9, 2.4 Hz, 1H), 6.22 (tt, J=55.6, 4.5 Hz, 1H), 4.71-4.60 (m, 2H), 3.80 (dtd, J=17.9, 9.3, 4.5 Hz, 2H), 3.70-3.62 (m, 2H), 3.58 (t, J=7.0 Hz, 2H), 3.51-3.34 (m, 2H), 2.31-2.06 (m, 4H). MS m/z: 454 [M+H]⁺.

### Example 473: 2-[1-(2,2,2-trifluoroethyl)-1H-pyrazolo[3,4-b]pyrazin-6-yl]-7-[2-(trifluoromethyl)pyridin-4-yl]-2,7-diazaspiro[4.4]nonane

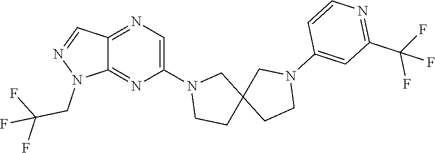 [SMILES: FC(F)(F)Cn1ncc2ncc(N3CCC4(CCN(c5ccnc(C(F)(F)F)c5)C4)C3)nc21]

Followed the General procedure A to afford the desired product as a white wax (20 mg, 78%). ¹H NMR (600 MHz, CDCl₃) δ 8.32 (d, J=5.8 Hz, 1H), 8.09 (s, 1H), 7.98 (s, 1H), 6.76 (d, J=2.4 Hz, 1H), 6.50 (dd, J=5.9, 2.4 Hz, 1H), 4.89 (dd, J=8.5, 2.1 Hz, 2H), 3.80 (dt, J=11.6, 4.4 Hz, 2H), 3.66 (d, J=4.0 Hz, 2H), 3.58 (t, J=7.0 Hz, 2H), 3.44 (q, J=9.9 Hz, 2H), 2.25-2.13 (m, 4H). MS m/z: 472 [M+H]⁺.

### Example 474: 2-[1-(oxetan-3-yl)-1H-pyrazolo[3,4-d]pyrimidin-6-yl]-7-[2-(trifluoromethyl)pyridin-4-yl]-2,7-diazaspiro[4.4]nonane

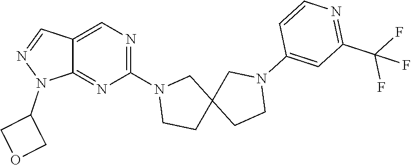 [SMILES: FC(F)(F)c1cc(N2CCC3(CCN(c4ncc5cnn(C6COC6)c5n4)C3)C2)ccn1]

Followed the General procedure A to afford the desired product as a colorless oil (2 mg, 4.87%). ¹H NMR (500 MHz, CDCl₃) δ 8.77 (s, 1H), 8.30 (d, J=5.9 Hz, 1H), 7.95 (s, 1H), 6.73 (s, 1H), 6.48 (s, 1H), 5.99-5.89 (m, 1H), 5.30 (s, 2H), 5.05 (t, J=7.3 Hz, 2H), 3.89-3.62 (m, 4H), 3.56 (d, J=7.2 Hz, 2H), 3.40 (q, J=9.8 Hz, 2H), 2.25-2.06 (m, 4H). MS m/z: 446 [M+H]⁺.

### Example 475: 2-[3-methyl-1-(oxetan-3-yl)-1H-pyrazolo[3,4-d]pyrimidin-6-yl]-7-[2-(trifluoromethyl)pyridin-4-yl]-2,7-diazaspiro[4.4]nonane

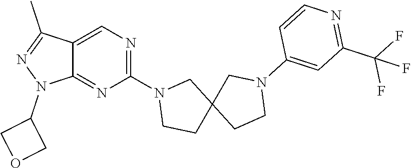 [SMILES: Cc1nn(C2COC2)c2nc(N3CCC4(CCN(c5ccnc(C(F)(F)F)c5)C4)C3)ncc12]

Followed the General procedure A to afford the desired product as a white wax (14 mg, 56%). ¹H NMR (600 MHz, CDCl₃) δ 8.66 (s, 1H), 8.30 (d, J=5.8 Hz, 1H), 6.85-6.61 (m, 1H), 6.53-6.32 (m, 1H), 5.87 (q, J=7.2 Hz, 1H), 5.28 (t, J=6.4 Hz, 2H), 5.01 (t, J=7.2 Hz, 2H), 3.87-3.61 (m, 4H), 3.54 (t, J=7.0 Hz, 2H), 3.49-3.25 (m, 2H), 2.52 (s, 3H), 2.22-1.99 (m, 4H). MS m/z: 460 [M+H]⁺.

### Example 476: 2-[3-methyl-1-(oxetan-3-yl)-1H-pyrazolo[3,4-b]pyrazin-6-yl]-7-[2-(trifluoromethyl)pyridin-4-yl]-2,7-diazaspiro[4.4]nonane

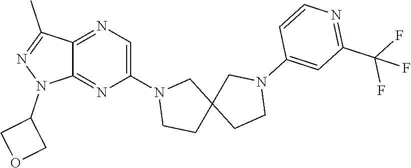 [SMILES: Cc1nn(C2COC2)c2nc(N3CCC4(CCN(c5ccnc(C(F)(F)F)c5)C4)C3)cnc12]

Followed the General procedure A to afford the desired product as a colorless oil (19 mg, 44.81%). ¹H NMR (500 MHz, CDCl₃) δ 8.31 (d, J=5.8 Hz, 1H), 7.87 (s, 1H), 6.75 (d, J=2.4 Hz, 1H), 6.49 (dd, J=6.0, 2.4 Hz, 1H), 5.88 (ddd, J=8.0, 6.8, 1.2 Hz, 1H), 5.32 (t, J=6.6 Hz, 2H), 5.02 (ddd, J=7.9, 6.7, 1.0 Hz, 2H), 3.78 (q, J=7.3 Hz, 2H), 3.68-3.60 (m, 2H), 3.58 (t, J=7.0 Hz, 2H), 3.47-3.38 (m, 2H), 2.59 (s, 3H), 2.26-2.12 (m, 4H). MS m/z: 460 [M+H]⁺.

### Example 477: 7-[1-(2,2,2-trifluoroethyl)-1H-pyrazolo[3,4-b]pyrazin-6-yl]-2-[4-(trifluoromethyl)pyridin-2-yl]-2,7-diazaspiro[3.5]nonane

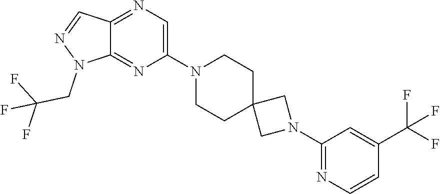 [SMILES: FC(F)(F)Cn1ncc2ncc(N3CCC4(CC3)CN(c3cc(C(F)(F)F)ccn3)C4)nc21]

Followed the General procedure A to afford the desired product as a white wax (14 mg, 14%). ¹H NMR (600 MHz, CDCl₃) δ 8.28 (d, J=6.3 Hz, 2H), 8.08 (s, 1H), 6.79 (dd, J=5.2, 1.4 Hz, 1H), 6.47 (s, 1H), 4.89 (q, J=8.4 Hz, 2H), 3.90 (s, 4H), 3.85-3.74 (m, 4H), 1.98 (dd, J=6.7, 4.6 Hz, 4H). MS m/z: 472 [M+H]⁺.

### Example 478: 7-[1-(oxetan-3-yl)-1H-pyrazolo[3,4-d]pyrimidin-6-yl]-2-[4-(trifluoromethyl)pyridin-2-yl]-2,7-diazaspiro[3.5]nonane

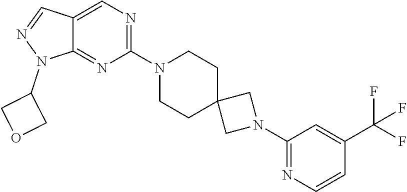 [SMILES: FC(F)(F)c1ccnc(N2CC3(CCN(c4ncc5cnn(C6COC6)c5n4)CC3)C2)c1]

Followed the General procedure A to afford the desired product as a white wax (8 mg, 33%). ¹H NMR (600 MHz, CDCl₃) δ 8.75 (s, 1H), 8.27 (d, J=5.3 Hz, 1H), 7.92 (s, 1H), 6.77 (dd, J=5.3, 1.5 Hz, 1H), 6.46 (s, 1H), 6.00-5.88 (m, 1H), 5.30 (t, J=6.6 Hz, 2H), 5.05 (dd, J=7.9, 6.6 Hz, 2H), 3.99-3.91 (m, 5H), 3.88 (s, 4H), 1.91 (dd, J=6.7, 4.5 Hz, 4H). MS m/z: 446 [M+H]⁺.

### Example 479: 7-[3-methyl-1-(oxetan-3-yl)-1H-pyrazolo[3,4-d]pyrimidin-6-yl]-2-[4-(trifluoromethyl)pyridin-2-yl]-2,7-diazaspiro[3.5]nonane

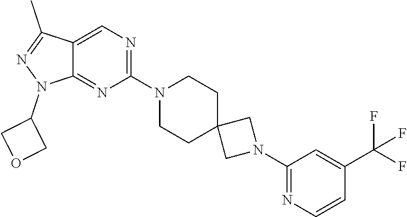 [SMILES: Cc1nn(C2COC2)c2nc(N3CCC4(CC3)CN(c3cc(C(F)(F)F)ccn3)C4)ncc12]

Followed the General procedure A to afford the desired product as a colorless oil (16 mg, 64%). ¹H NMR (600 MHz, CDCl₃) δ 8.65 (s, 1H), 8.27 (d, J=5.3 Hz, 1H), 6.76 (dd, J=5.3, 1.5 Hz, 1H), 6.46 (s, 1H), 5.90-5.80 (m, 1H), 5.28 (t, J=6.6 Hz, 2H), 5.02 (dd, J=7.9, 6.5 Hz, 2H), 3.94-3.91 (m, 4H), 3.88 (s, 4H), 2.52 (s, 3H), 1.91 (dd, J=6.7, 4.4 Hz, 4H). MS m/z: 460 [M+H]⁺.

### Example 480: 7-[3-methyl-1-(oxetan-3-yl)-1H-pyrazolo[3,4-b]pyrazin-6-yl]-2-[4-(trifluoromethyl)pyridin-2-yl]-2,7-diazaspiro[3.5]nonane

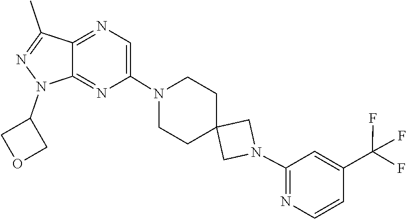 [SMILES: Cc1nn(C2COC2)c2nc(N3CCC4(CC3)CN(c3cc(C(F)(F)F)ccn3)C4)cnc12]

Followed the General procedure A to afford the desired product as a white wax (14 mg, 56%). ¹H NMR (600 MHz, CDCl₃) δ 8.28 (d, J=5.3 Hz, 1H), 8.17 (s, 1H), 6.78 (dd, J=5.3, 1.5 Hz, 1H), 6.46 (s, 1H), 5.92-5.83 (m, 1H), 5.32 (t, J=6.6 Hz, 2H), 5.04 (dd, J=8.0, 6.5 Hz, 2H), 3.89 (s, 4H), 3.77-3.74 (m, 4H), 2.59 (s, 3H), 1.99-1.96 (m, 4H). MS m/z: 460 [M+H]⁺.

### Example 481: 2-[1-(2,2-difluoroethyl)-1H-pyrazolo[3,4-b]pyrazin-6-yl]-7-[2-(trifluoromethyl)pyrimidin-5-yl]-2,7-diazaspiro[4.4]nonane

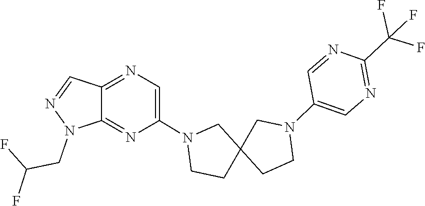 [SMILES: FC(F)Cn1ncc2ncc(N3CCC4(CCN(c5cnc(C(F)(F)F)nc5)C4)C3)nc21]

Followed the General procedure A to afford the desired product as a colorless oil (124 mg, 60.03%). ¹H NMR (500 MHz, CDCl₃) δ 8.12 (s, 2H), 8.06 (s, 1H), 7.97 (s, 1H), 6.22 (tt, J=55.6, 4.5 Hz, 1H), 4.66 (td, J=13.3, 4.5 Hz, 2H), 3.87-3.76 (m, 2H), 3.72-3.65 (m, 2H), 3.62 (t, J=7.0 Hz, 2H), 3.46 (q, J=9.6 Hz, 2H), 2.30-2.14 (m, 4H). MS m/z: 455 [M+H]⁺.

### Example 482: 2-[1-(2,2-difluoroethyl)-1H-pyrazolo[3,4-b]pyrazin-6-yl]-7-[6-(trifluoromethyl)pyridin-3-yl]-2,7-diazaspiro[4.4]nonane

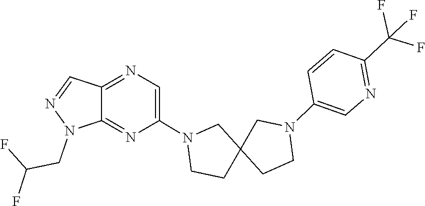 [SMILES: FC(F)Cn1ncc2ncc(N3CCC4(CCN(c5ccc(C(F)(F)F)nc5)C4)C3)nc21]

Followed the General procedure A to afford the desired product as a white wax (13 mg, 50%). ¹H NMR (600 MHz, CDCl₃) δ 8.11-8.00 (m, 2H), 7.97 (s, 1H), 7.50 (d, J=8.6 Hz, 1H), 6.85 (dd, J=8.7, 2.9 Hz, 1H), 6.22 (tt, J=55.7, 4.5 Hz, 1H), 4.66 (td, J=13.4, 4.5 Hz, 2H), 3.85-3.74 (m, 2H), 3.67 (q, J=10.9 Hz, 2H), 3.58 (t, J=6.9 Hz, 2H), 3.50-3.37 (m, 2H), 2.25-2.13 (m, 4H). MS m/z: 454 [M+H]⁺.

### Example 483: 2-[1-(2,2-difluoroethyl)-1H-pyrazolo[3,4-b]pyrazin-6-yl]-7-[5-(trifluoromethyl)pyridin-2-yl]-2,7-diazaspiro[4.4]nonane

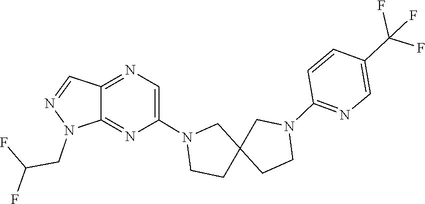 [SMILES: FC(F)Cn1ncc2ncc(N3CCC4(CCN(c5ccc(C(F)(F)F)cn5)C4)C3)nc21]

Followed the General procedure A to afford the desired product as a colorless oil (16 mg, 62%). ¹H NMR (600 MHz, CDCl₃) δ 8.39 (d, J=2.4 Hz, 1H), 8.05 (s, 1H), 7.96 (s, 1H), 7.63 (dd, J=8.9, 2.5 Hz, 1H), 6.40 (d, J=8.9 Hz, 1H), 6.22 (tt, J=55.7, 4.6 Hz, 1H), 4.66 (tdd, J=13.3, 4.5, 1.6 Hz, 2H), 3.78 (q, J=6.7 Hz, 2H), 3.71-3.54 (m, 6H), 2.23-2.10 (m, 4H). MS m/z: 454 [M+H]⁺.

### Example 484: 2-[1-(2,2-difluoroethyl)-1H-pyrazolo[3,4-b]pyrazin-6-yl]-7-[2-(trifluoromethyl)pyrimidin-4-yl]-2,7-diazaspiro[4.4]nonane

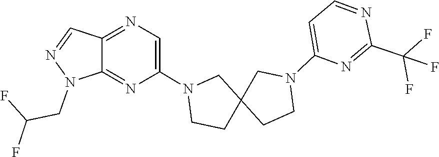 [SMILES: FC(F)Cn1ncc2ncc(N3CCC4(CCN(c5ccnc(C(F)(F)F)n5)C4)C3)nc21]

Followed the General procedure A to afford the desired product as a colorless oil (18 mg, 70%). ¹H NMR (600 MHz, CDCl₃) δ 8.31 (d, J=7.5 Hz, 1H), 8.05 (s, 1H), 7.96 (s, 1H), 6.40 (d, J=47.0 Hz, 1H), 6.22 (tt, J=55.6, 4.5 Hz, 1H), 4.66 (td, J=13.3, 4.5 Hz, 2H), 3.95-3.37 (m, 8H), 2.21 (dt, J=13.4, 6.6 Hz, 4H). MS m/z: 455 [M+H]⁺.

### Example 485: 2-[1-(2,2-difluoroethyl)-3-methyl-1H-pyrazolo[3,4-b]pyrazin-6-yl]-7-[2-(trifluoromethyl)pyridin-4-yl]-2,7-diazaspiro[4.4]nonane

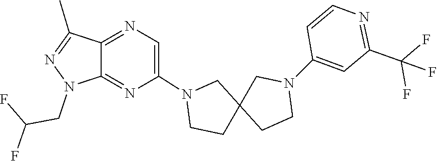 [SMILES: Cc1nn(CC(F)F)c2nc(N3CCC4(CCN(c5ccnc(C(F)(F)F)c5)C4)C3)cnc12]

Followed the General procedure A to afford the desired product as a white wax (16 mg, 63%). ¹H NMR (600 MHz, CDCl₃) δ 8.31 (d, J=5.8 Hz, 1H), 7.89 (s, 1H), 6.75 (d, J=2.4 Hz, 1H), 6.50 (dd, J=5.8, 2.5 Hz, 1H), 6.20 (tt, J=55.8, 4.5 Hz, 1H), 4.65-4.52 (m, 2H), 3.84-3.74 (m, 2H), 3.69-3.62 (m, 2H), 3.58 (t, J=7.0 Hz, 2H), 3.47-3.39 (m, 2H), 2.55 (s, 3H), 2.25-2.12 (m, 4H). MS m/z: 468 [M+H]⁺.

### Example 486: 2-[1-(2,2-difluoroethyl)-3-methyl-1H-pyrazolo[3,4-d]pyrimidin-6-yl]-7-[2-(trifluoromethyl)pyridin-4-yl]-2,7-diazaspiro[4.4]nonane

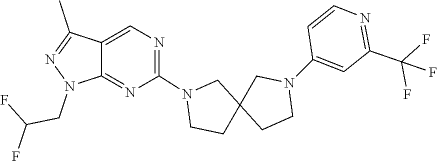 [SMILES: Cc1nn(CC(F)F)c2nc(N3CCC4(CCN(c5ccnc(C(F)(F)F)c5)C4)C3)ncc12]

Followed the General procedure A to afford the desired product as a white foam (14 mg, 55%). ¹H NMR (600 MHz, CDCl₃) δ 8.67 (s, 1H), 8.30 (d, J=5.8 Hz, 1H), 6.74 (s, 1H), 6.52-6.45 (m, 1H), 6.20 (t, J=55.7 Hz, 1H), 4.54 (dt, J=13.8, 8.2 Hz, 2H), 3.89-3.62 (m, 4H), 3.55 (t, J=7.0 Hz, 2H), 3.48-3.35 (m, 2H), 2.48 (s, 3H), 2.24-2.06 (m, 4H). MS m/z: 468 [M+H]⁺.

### Example 487: 2-[1-(oxetan-3-yl)-1H-pyrazolo[3,4-b]pyrazin-6-yl]-7-[2-(trifluoromethyl)pyridin-4-yl]-2,7-diazaspiro[4.4]nonane

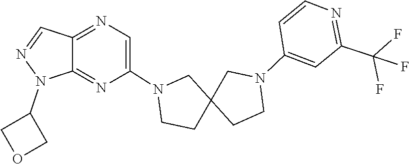 [SMILES: FC(F)(F)c1cc(N2CCC3(CCN(c4cnc5cnn(C6COC6)c5n4)C3)C2)ccn1]

Followed the General procedure A to afford the desired product as a colorless oil (14 mg, 58%). ¹H NMR (600 MHz, CDCl₃) δ 8.32 (d, J=5.8 Hz, 1H), 8.10 (s, 1H), 7.95 (s, 1H), 6.75 (d, J=2.4 Hz, 1H), 6.49 (dd, J=5.9, 2.5 Hz, 1H), 6.00-5.91 (m, 1H), 5.34 (t, J=6.6 Hz, 2H), 5.09-5.02 (m, 2H), 3.88-3.75 (m, 2H), 3.66 (q, J=10.9 Hz, 2H), 3.58 (t, J=7.0 Hz, 2H), 3.43 (q, J=9.9 Hz, 2H), 2.25-2.13 (m, 4H). MS m/z: 446 [M+H]⁺.

### Example 488: 2-[1-(2,2-difluoroethyl)-1H-pyrazolo[3,4-d]pyrimidin-6-yl]-7-[2-(trifluoromethyl)pyridin-4-yl]-2,7-diazaspiro[4.4]nonane

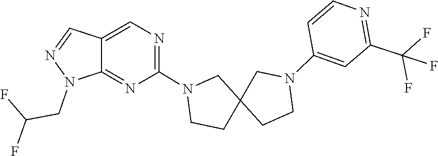 [SMILES: FC(F)Cn1ncc2cnc(N3CCC4(CCN(c5ccnc(C(F)(F)F)c5)C4)C3)nc21]

Followed the General procedure A to afford the desired product as a colorless oil (6 mg, 24%). ¹H NMR (600 MHz, CDCl₃) δ 8.77 (s, 1H), 8.30 (d, J=5.8 Hz, 1H), 7.90 (s, 1H), 6.74 (s, 1H), 6.48 (s, 1H), 6.23 (t, J=55.6 Hz, 1H), 4.63 (t, J=12.6 Hz, 2H), 3.76 (d, J=100.8 Hz, 4H), 3.56 (d, J=7.6 Hz, 2H), 3.45-3.36 (m, 2H), 2.26-2.06 (m, 4H). MS m/z: 454 [M+H]⁺.

### Example 489: 2-[1-(2,2,2-trifluoroethyl)-1H-pyrazolo[3,4-d]pyrimidin-6-yl]-7-[2-(trifluoromethyl)pyridin-4-yl]-2,7-diazaspiro[4.4]nonane

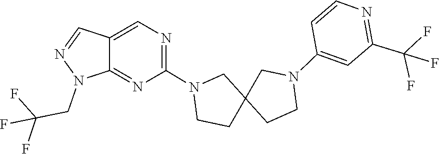 [SMILES: FC(F)(F)Cn1ncc2cnc(N3CCC4(CCN(c5ccnc(C(F)(F)F)c5)C4)C3)nc21]

Followed the General procedure A to afford the desired product as a colorless oil (5.6 mg, 12.87%). ¹H NMR (500 MHz, CDCl₃) δ 8.78 (s, 1H), 8.30 (d, J=5.8 Hz, 1H), 7.94 (s, 1H), 6.74 (s, 1H), 6.48 (s, 1H), 4.86 (br, 2H), 3.92-3.64 (m, 4H), 3.55 (s, 2H), 3.41 (q, J=9.8 Hz, 2H), 2.24-2.08 (m, 4H). MS m/z: 472 [M+H]⁺.

### Example 490: 7-[1-(2,2-difluoroethyl)-3-methyl-1H-pyrazolo[3,4-b]pyrazin-6-yl]-2-[4-(trifluoromethyl)pyridin-2-yl]-2,7-diazaspiro[3.5]nonane

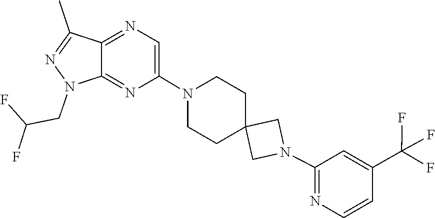 [SMILES: Cc1nn(CC(F)F)c2nc(N3CCC4(CC3)CN(c3cc(C(F)(F)F)ccn3)C4)cnc12]

Followed the General procedure A to afford the desired product as a white solid (14 mg, 55%). ¹H NMR (600 MHz, CDCl₃) δ 8.28 (d, J=5.3 Hz, 1H), 8.18 (s, 1H), 6.78 (dd, J=5.3, 1.5 Hz, 1H), 6.46 (s, 1H), 6.19 (tt, J=55.7, 4.5 Hz, 1H), 4.59 (td, J=13.4, 4.5 Hz, 2H), 3.89 (s, 4H), 3.78-3.74 (m, 4H), 2.55 (s, 3H), 1.99-1.96 (m, 4H). MS m/z: 468 [M+H]⁺.

### Example 491: 7-[1-(2,2-difluoroethyl)-3-methyl-1H-pyrazolo[3,4-d]pyrimidin-6-yl]-2-[4-(trifluoromethyl)pyridin-2-yl]-2,7-diazaspiro[3.5]nonane

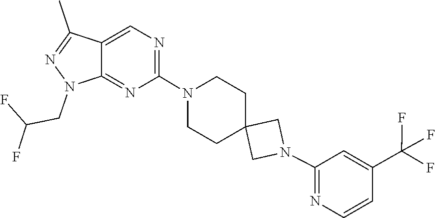 [SMILES: Cc1nn(CC(F)F)c2nc(N3CCC4(CC3)CN(c3cc(C(F)(F)F)ccn3)C4)ncc12]

Followed the General procedure A to afford the desired product as a white solid (20 mg, 79%). ¹H NMR (600 MHz, CDCl₃) δ 8.66 (s, 1H), 8.27 (d, J=5.3 Hz, 1H), 6.76 (dd, J=5.3, 1.5 Hz, 1H), 6.46 (s, 1H), 6.19 (tt, J=55.7, 4.5 Hz, 1H), 4.54 (td, J=13.4, 4.5 Hz, 2H), 3.96-3.91 (m, 4H), 3.88 (s, 4H), 2.48 (s, 3H), 1.94-1.88 (m, 4H). MS m/z: 468 [M+H]⁺.

### Example 492: 7-[1-(2,2-difluoroethyl)-1H-pyrazolo[3,4-d]pyrimidin-6-yl]-2-[4-(trifluoromethyl)pyridin-2-yl]-2,7-diazaspiro[3.5]nonane

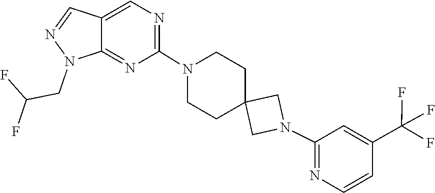 [SMILES: FC(F)Cn1ncc2cnc(N3CCC4(CC3)CN(c3cc(C(F)(F)F)ccn3)C4)nc21]

Followed the General procedure A to afford the desired product as a white solid (8 mg, 32%). ¹H NMR (600 MHz, CDCl₃) δ 8.75 (s, 1H), 8.27 (d, J=5.3 Hz, 1H), 7.87 (s, 1H), 6.77 (dd, J=5.3, 1.5 Hz, 1H), 6.46 (s, 1H), 6.22 (tt, J=55.6, 4.5 Hz, 1H), 4.63 (td, J=13.4, 4.5 Hz, 2H), 4.00-3.92 (m, 4H), 3.89 (s, 4H), 1.97-1.87 (m, 4H). MS m/z: 454 [M+H]⁺.

### Example 493: 7-[1-(oxetan-3-yl)-1H-pyrazolo[3,4-b]pyrazin-6-yl]-2-[4-(trifluoromethyl)pyridin-2-yl]-2,7-diazaspiro[3.5]nonane

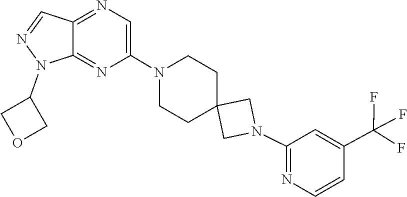 [SMILES: FC(F)(F)c1ccnc(N2CC3(CCN(c4cnc5cnn(C6COC6)c5n4)CC3)C2)c1]

Followed the General procedure A to afford the desired product as a white solid (15 mg, 52%). ¹H NMR (600 MHz, CDCl₃) δ 8.31-8.22 (m, 2H), 8.09 (s, 1H), 6.79 (dd, J=5.3, 1.6 Hz, 1H), 6.47 (s, 1H), 5.94 (tt, J=7.8, 6.6 Hz, 1H), 5.34 (t, J=6.6 Hz, 2H), 5.06 (dd, J=7.9, 6.6 Hz, 2H), 3.90 (s, 4H), 3.82-3.74 (m, 4H), 2.03-1.96 (m, 4H). MS m/z: 446 [M+H]⁺.

### Example 494: 7-[1-(2,2-difluoroethyl)-1H-pyrazolo[3,4-b]pyrazin-6-yl]-2-[2-(trifluoromethyl)pyridin-4-yl]-2,7-diazaspiro[4.4]nonan-3-one

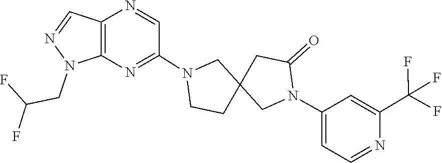 [SMILES: O=C1CC2(CCN(c3cnc4cnn(CC(F)F)c4n3)C2)CN1c1ccnc(C(F)(F)F)c1]

Followed the General procedure A to afford the desired product as a light yellow oil (16 mg, 63%). ¹H NMR (500 MHz, CDCl₃) δ 8.67 (d, J=5.6 Hz, 1H), 8.08 (s, 1H), 7.99 (s, 1H), 7.96 (d, J=2.2 Hz, 1H), 7.85 (dd, J=5.7, 2.1 Hz, 1H), 6.22 (tt, J=55.6, 4.5 Hz, 2H), 4.67 (td, J=13.4, 4.5 Hz, 2H), 3.92 (q, J=9.8 Hz, 2H), 3.87-3.72 (m, 4H), 2.90-2.77 (m, 2H), 2.29 (dt, J=7.2, 4.1 Hz, 2H). MS m/z: 468 [M+H]⁺.

### Example 495: 7-[1-(2,2-difluoroethyl)-3-methyl-1H-pyrazolo[3,4-b]pyrazin-6-yl]-2-[2-(trifluoromethyl)pyridin-4-yl]-2,7-diazaspiro[4.4]nonan-3-one

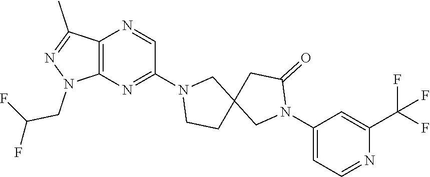 [SMILES: Cc1nn(CC(F)F)c2nc(N3CCC4(CC(=O)N(c5ccnc(C(F)(F)F)c5)C4)C3)cnc12]

Followed the General procedure A to afford the desired product as a colorless oil (15 mg, 57%). ¹H NMR (500 MHz, CDCl₃) δ 8.67 (d, J=5.6 Hz, 1H), 7.96 (d, J=2.2 Hz, 1H), 7.91 (s, 1H), 7.85 (dd, J=5.7, 2.2 Hz, 1H), 6.19 (tt, J=55.7, 4.5 Hz, 1H), 4.59 (td, J=13.4, 4.5 Hz, 2H), 3.96-3.85 (m, 2H), 3.85-3.68 (m, 4H), 2.90-2.76 (m, 2H), 2.28 (td, J=7.1, 2.1 Hz, 2H). MS m/z: 482 [M+H]⁺.

### Example 496: 7-[1-(2,2-difluoroethyl)-3-methyl-1H-pyrazolo[3,4-d]pyrimidin-6-yl]-2-[2-(trifluoromethyl)pyridin-4-yl]-2,7-diazaspiro[4.4]nonan-3-one

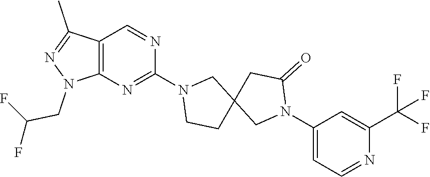 [SMILES: Cc1nn(CC(F)F)c2nc(N3CCC4(CC(=O)N(c5ccnc(C(F)(F)F)c5)C4)C3)ncc12]

Followed the General procedure A to afford the desired product as a colorless oil (17.6 mg, 67%). ¹H NMR (500 MHz, CDCl₃) δ 8.68 (s, 1H), 8.66 (d, J=5.6 Hz, 1H), 7.98-7.92 (m, 1H), 7.84 (dd, J=5.7, 2.1 Hz, 1H), 6.19 (t, J=55.6 Hz, 1H), 4.55 (t, J=12.6 Hz, 2H), 3.93-3.70 (m, 6H), 2.87-2.74 (m, 2H), 2.21 (dd, J=7.7, 6.3 Hz, 2H). MS m/z: 482 [M+H]⁺.

### Example 497: 7-[1-(2,2-difluoroethyl)-1H-pyrazolo[3,4-b]pyrazin-6-yl]-2-[5-(trifluoromethyl)pyridin-2-yl]-2,7-diazaspiro[4.4]nonan-3-one

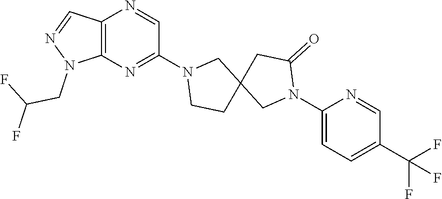 [SMILES: O=C1CC2(CCN(c3cnc4cnn(CC(F)F)c4n3)C2)CN1c1ccc(C(F)(F)F)cn1]

Followed the General procedure A to afford the desired product as a colorless oil (16 mg, 62%). ¹H NMR (500 MHz, CDCl₃) δ 8.62-8.54 (m, 2H), 8.06 (s, 1H), 7.97 (s, 1H), 7.94 (dd, J=9.0, 2.3 Hz, 1H), 6.22 (tt, J=55.6, 4.5 Hz, 1H), 4.66 (td, J=13.3, 4.5 Hz, 2H), 4.17 (q, J=11.5 Hz, 2H), 3.88-3.68 (m, 4H), 2.90-2.79 (m, 2H), 2.25 (hept, J=7.2, 6.7 Hz, 2H). MS m/z: 468 [M+H]⁺.

### Example 498: 7-[1-(2,2-difluoroethyl)-1H-pyrazolo[3,4-b]pyrazin-6-yl]-2-[6-(trifluoromethyl)pyridin-3-yl]-2,7-diazaspiro[4.4]nonan-3-one

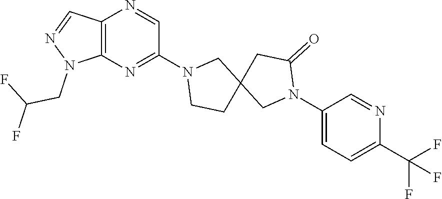 [SMILES: O=C1CC2(CCN(c3cnc4cnn(CC(F)F)c4n3)C2)CN1c1ccc(C(F)(F)F)nc1]

Followed the General procedure A to afford the desired product as a colorless oil (17.5 mg, 69%). ¹H NMR (500 MHz, CDCl₃) δ 8.80 (d, J=2.6 Hz, 1H), 8.48 (dd, J=8.7, 2.6 Hz, 1H), 8.08 (s, 1H), 7.99 (s, 1H), 7.72 (d, J=8.6 Hz, 1H), 6.22 (tt, J=55.6, 4.5 Hz, 1H), 4.67 (td, J=13.3, 4.5 Hz, 2H), 3.95 (q, J=9.7 Hz, 2H), 3.87-3.72 (m, 4H), 2.88-2.76 (m, 2H), 2.30 (td, J=6.9, 3.0 Hz, 2H). MS m/z: 468 [M+H]⁺.

### Example 499: 2-[2-methyl-6-(trifluoromethyl)pyrimidin-4-yl]-6-[1-(2,2,2-trifluoroethyl)-1H-pyrazolo[3,4-b]pyrazin-6-yl]-2,6-diazaspiro[3.4]octane

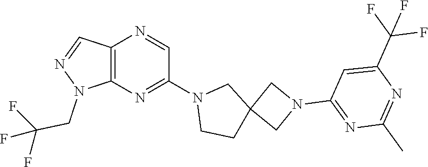 [SMILES: Cc1nc(N2CC3(CCN(c4cnc5cnn(CC(F)(F)F)c5n4)C3)C2)cc(C(F)(F)F)n1]

Followed the General procedure A to afford the desired product as a colorless oil (15 mg, 58%). ¹H NMR (500 MHz, CDCl₃) δ 8.10 (s, 1H), 7.98 (s, 1H), 6.35 (s, 1H), 4.89 (q, J=8.4 Hz, 2H), 4.19 (s, 4H), 3.90 (s, 2H), 3.76 (t, J=6.9 Hz, 2H), 2.58 (s, 3H), 2.40 (t, J=6.9 Hz, 2H). MS m/z: 473 [M+H]⁺.

### Example 500: 2-[2-methyl-6-(trifluoromethyl)pyrimidin-4-yl]-6-[1-(2,2,2-trifluoroethyl)-1H-pyrazolo[3,4-d]pyrimidin-6-yl]-2,6-diazaspiro[3.4]octane

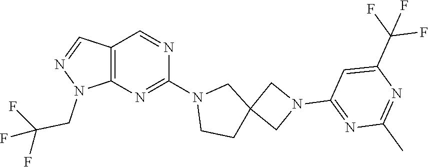 [SMILES: Cc1nc(N2CC3(CCN(c4ncc5cnn(CC(F)(F)F)c5n4)C3)C2)cc(C(F)(F)F)n1]

Followed the General procedure A to afford the desired product as a colorless oil (6.5 mg, 25%). ¹H NMR (500 MHz, CDCl₃) δ 8.77 (s, 1H), 7.92 (s, 1H), 6.32 (s, 1H), 4.85 (q, J=8.4 Hz, 2H), 4.13 (s, 4H), 3.90 (s, 2H), 3.77 (s, 2H), 2.55 (s, 3H), 2.31 (t, J=6.9 Hz, 2H). MS m/z: 473 [M+H]⁺.

### Example 501: 6-[1-(2,2-difluoroethyl)-1H-pyrazolo[3,4-d]pyrimidin-6-yl]-2-[2-methyl-6-(trifluoromethyl)pyrimidin-4-yl]-2,6-diazaspiro[3.4]octane

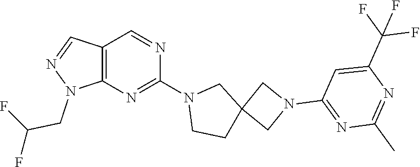 [SMILES: Cc1nc(N2CC3(CCN(c4ncc5cnn(CC(F)F)c5n4)C3)C2)cc(C(F)(F)F)n1]

Followed the General procedure A to afford the desired product as a colorless oil (5.7 mg, 23%). ¹H NMR (500 MHz, CDCl₃) δ 8.81 (s, 1H), 7.93 (s, 1H), 7.29 (s, 1H), 6.36 (d, J=6.8 Hz, 1H), 6.22 (dd, J=32.2, 27.8 Hz, 1H), 4.66 (td, J=13.3, 4.4 Hz, 2H), 4.18 (s, 4H), 3.94 (s, 2H), 3.81 (s, 2H), 2.60 (s, 3H), 2.35 (t, J=6.9 Hz, 2H). MS m/z: 455 [M+H]⁺.

### Example 502: 6-[1-(2,2-difluoroethyl)-3-methyl-1H-pyrazolo[3,4-d]pyrimidin-6-yl]-2-[2-methyl-6-(trifluoromethyl)pyrimidin-4-yl]-2,6-diazaspiro[3.4]octane

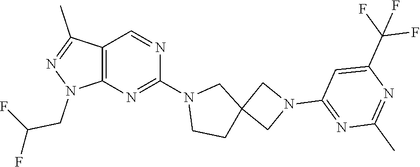 [SMILES: Cc1nc(N2CC3(CCN(c4ncc5c(C)nn(CC(F)F)c5n4)C3)C2)cc(C(F)(F)F)n1]

Followed the General procedure A to afford the desired product as a white foam (18.4 mg, 72%). ¹H NMR (500 MHz, CDCl₃) δ 8.68 (s, 1H), 6.34 (s, 1H), 6.20 (tt, J=55.7, 4.5 Hz, 1H), 4.55 (td, J=13.4, 4.5 Hz, 2H), 4.15 (s, 4H), 3.91 (s, 2H), 3.77 (s, 2H), 2.57 (s, 3H), 2.49 (s, 3H), 2.32 (t, J=6.9 Hz, 2H). MS m/z: 469 [M+H]⁺.

### Example 503: 6-[1-(2,2-difluoroethyl)-3-methyl-1H-pyrazolo[3,4-b]pyrazin-6-yl]-2-[2-methyl-6-(trifluoromethyl)pyrimidin-4-yl]-2,6-diazaspiro[3.4]octane

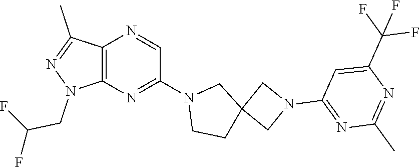 [SMILES: Cc1nc(N2CC3(CCN(c4cnc5c(C)nn(CC(F)F)c5n4)C3)C2)cc(C(F)(F)F)n1]

Followed the General procedure A to afford the desired product as a colorless oil (16 mg, 63%). ¹H NMR (500 MHz, CDCl₃) δ 7.89 (s, 1H), 6.35 (s, 1H), 6.20 (tt, J=55.8, 4.5 Hz, 1H), 4.59 (td, J=13.4, 4.5 Hz, 2H), 4.19 (s, 4H), 3.88 (s, 2H), 3.74 (t, J=6.9 Hz, 2H), 2.58 (s, 3H), 2.55 (s, 3H), 2.39 (t, J=6.9 Hz, 2H). MS m/z: 469 [M+H]⁺.

### Example 504: 6-[3-methyl-1-(oxetan-3-yl)-1H-pyrazolo[3,4-d]pyrimidin-6-yl]-2-[2-methyl-6-(trifluoromethyl)pyrimidin-4-yl]-2,6-diazaspiro[3.4]octane

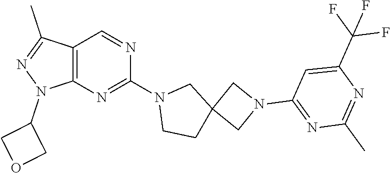 [SMILES: Cc1nc(N2CC3(CCN(c4ncc5c(C)nn(C6COC6)c5n4)C3)C2)cc(C(F)(F)F)n1]

Followed the General procedure A to afford the desired product as a colorless oil (20 mg, 80%). ¹H NMR (500 MHz, CDCl₃) δ 8.67 (s, 1H), 6.33 (s, 1H), 5.87 (p, J=7.3 Hz, 1H), 5.29 (t, J=6.6 Hz, 2H), 5.03 (dd, J=7.9, 6.5 Hz, 2H), 4.14 (s, 4H), 3.90 (s, 2H), 3.76 (s, 2H), 2.57 (s, 3H), 2.53 (s, 3H), 2.31 (t, J=6.9 Hz, 2H). MS m/z: 461 [M+H]⁺.

### Example 505: 2-[2-methyl-6-(trifluoromethyl)pyrimidin-4-yl]-6-[6-(1,3,4-thiadiazol-2-yl)pyrazin-2-yl]-2,6-diazaspiro[3.4]octane

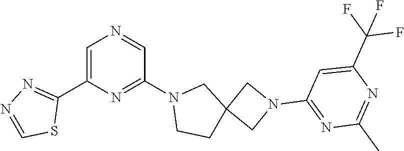 [SMILES: Cc1nc(N2CC3(CCN(c4cncc(-c5nncs5)n4)C3)C2)cc(C(F)(F)F)n1]

Followed the General procedure A to afford the desired product as a light yellow oil (16 mg, 67%). ¹H NMR (500 MHz, CDCl₃) δ 9.20 (s, 1H), 8.88 (s, 1H), 8.01 (s, 1H), 6.35 (s, 1H), 4.19 (s, 4H), 3.85 (s, 2H), 3.70 (t, J=6.9 Hz, 2H), 2.58 (s, 3H), 2.40 (t, J=6.9 Hz, 2H). MS m/z: 435 [M+H]⁺.

### Example 506: 2-[1-(2,2-difluoroethyl)-1H-pyrazolo[3,4-b]pyrazin-6-yl]-7-[2-(trifluoromethyl)pyridin-4-yl]-2,7-diazaspiro[4.4]nonan-3-one

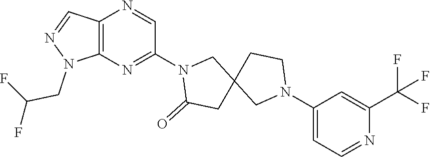 [SMILES: O=C1CC2(CCN(c3ccnc(C(F)(F)F)c3)C2)CN1c1cnc2cnn(CC(F)F)c2n1]

Followed the General procedure A to afford the desired product as a white solid (14 mg, 8%). ¹H NMR (500 MHz, CDCl₃) δ 9.78 (s, 1H), 8.30 (d, J=5.8 Hz, 1H), 8.26 (s, 1H), 6.75 (d, J=2.4 Hz, 1H), 6.49 (dd, J=6.0, 2.4 Hz, 1H), 6.19 (tt, J=55.3, 4.4 Hz, 1H), 4.73 (tdd, J=13.4, 4.4, 3.2 Hz, 2H), 4.13 (d, J=1.3 Hz, 2H), 3.71-3.56 (m, 2H), 3.55 (d, J=9.9 Hz, 1H), 3.48 (d, J=9.9 Hz, 1H), 2.85 (d, J=3.4 Hz, 2H), 2.25 (ddt, J=19.9, 12.7, 7.2 Hz, 2H). MS m/z: 468 [M+H]⁺.

### Example 507: 2-[1-(2,2-difluoroethyl)-1H-pyrazolo[3,4-b]pyrazin-6-yl]-7-[6-(trifluoromethyl)pyridin-3-yl]-2,7-diazaspiro[4.4]nonan-3-one

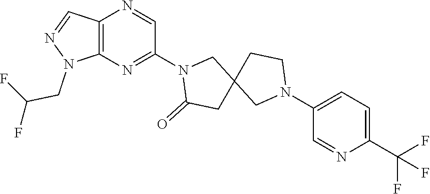 [SMILES: O=C1CC2(CCN(c3ccc(C(F)(F)F)nc3)C2)CN1c1cnc2cnn(CC(F)F)c2n1]

Followed the General procedure A to afford the desired product as a white solid (10.5 mg, 29%). ¹H NMR (500 MHz, CDCl₃) δ 9.83 (s, 1H), 8.30 (s, 1H), 8.06 (d, J=2.8 Hz, 1H), 7.52 (d, J=8.7 Hz, 1H), 6.88 (dd, J=8.7, 2.9 Hz, 1H), 6.22 (tt, J=55.4, 4.4 Hz, 2H), 4.76 (ddt, J=17.3, 13.4, 3.7 Hz, 2H), 4.20-4.12 (m, 2H), 3.69-3.59 (m, 2H), 3.59-3.48 (m, 2H), 2.94-2.83 (m, 2H), 2.28 (ddt, J=19.9, 12.6, 7.2 Hz, 2H). MS m/z: 468 [M+H]⁺.

### Example 508: 2-[1-(2,2-difluoroethyl)-1H-pyrazolo[3,4-b]pyrazin-6-yl]-7-[5-(trifluoromethyl)pyridin-2-yl]-2,7-diazaspiro[4.4]nonan-3-one

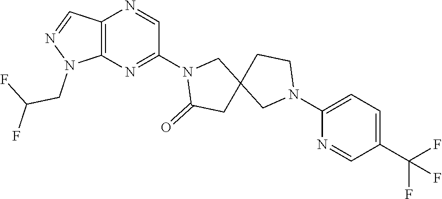 [SMILES: O=C1CC2(CCN(c3ccc(C(F)(F)F)cn3)C2)CN1c1cnc2cnn(CC(F)F)c2n1]

Followed the General procedure A to afford the desired product as a white solid (11 mg, 30%). ¹H NMR (500 MHz, CDCl₃) δ 9.82 (s, 1H), 8.40 (t, J=1.7 Hz, 1H), 8.29 (s, 1H), 7.66 (dd, J=8.9, 2.5 Hz, 1H), 6.43 (d, J=8.9 Hz, 1H), 6.22 (tt, J=55.3, 4.4 Hz, 1H), 4.76 (tt, J=13.5, 4.5 Hz, 2H), 4.14 (s, 2H), 3.80-3.59 (m, 4H), 2.95-2.79 (m, 2H), 2.32-2.18 (m, 2H). MS m/z: 468 [M+H]⁺.

### Example 509: 2-[1-(2,2-difluoroethyl)-1H-pyrazolo[3,4-b]pyrazin-6-yl]-7-[2-methyl-6-(trifluoromethyl)pyrimidin-4-yl]-2,7-diazaspiro[4.4]nonan-3-one

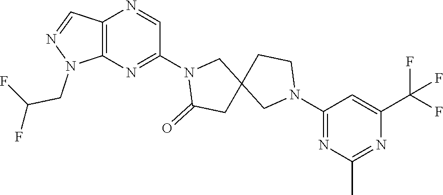 [SMILES: Cc1nc(N2CCC3(CC(=O)N(c4cnc5cnn(CC(F)F)c5n4)C3)C2)cc(C(F)(F)F)n1]

Followed the General procedure A to afford the desired product as a white solid (11.3 mg, 30%). ¹H NMR (500 MHz, CDCl₃) δ 9.82 (s, 1H), 8.30 (s, 1H), 6.48 (d, J=38.1 Hz, 1H), 6.36-6.05 (m, 2H), 4.76 (tt, J=13.5, 3.8 Hz, 2H), 4.13 (s, 2H), 3.95-3.74 (m, 2H), 3.59 (d, J=47.5 Hz, 2H), 2.86 (d, J=12.2 Hz, 2H), 2.59 (d, J=17.2 Hz, 3H), 2.30 (d, J=19.2 Hz, 3H). MS m/z: 483 [M+H]⁺.

### Example 510: 7-[1-(2,2-difluoroethyl)-1H-pyrazolo[3,4-b]pyrazin-6-yl]-2-[2-methyl-6-(trifluoromethyl)pyrimidin-4-yl]-2,7-diazaspiro[4.4]nonan-3-one

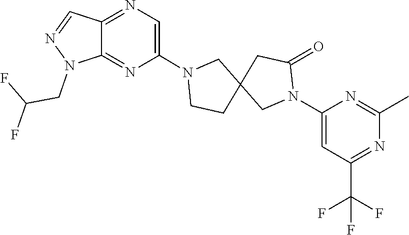 [SMILES: Cc1nc(N2CC3(CCN(c4cnc5cnn(CC(F)F)c5n4)C3)CC2=O)cc(C(F)(F)F)n1]

Followed the General procedure A to afford the desired product as a white solid (12.3 mg, 35%). ¹H NMR (500 MHz, CDCl₃) δ 8.58 (s, 1H), 8.07 (s, 1H), 7.98 (s, 1H), 6.22 (tt, J=55.6, 4.5 Hz, 1H), 4.66 (td, J=13.3, 4.5 Hz, 2H), 4.14 (q, J=11.7 Hz, 2H), 3.84 (q, J=6.3, 5.6 Hz, 2H), 3.81-3.68 (m, 2H), 2.92-2.79 (m, 2H), 2.71 (s, 3H), 2.25 (dq, J=16.6, 6.1, 5.7 Hz, 2H). MS m/z: 483 [M+H]⁺.

### Example 511: 8-[1-(2,2-difluoroethyl)-1H-pyrazolo[3,4-b]pyrazin-6-yl]-2-[2-methyl-6-(trifluoromethyl)pyrimidin-4-yl]-2,8-diazaspiro[4.5]decane

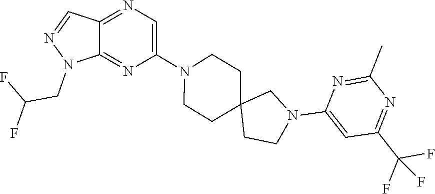 [SMILES: Cc1nc(N2CCC3(CCN(c4cnc5cnn(CC(F)F)c5n4)CC3)C2)cc(C(F)(F)F)n1]

Followed the General procedure A to afford the desired product as a white solid (27 mg, 67%). ¹H NMR (500 MHz, CDCl₃) δ 8.25 (s, 1H), 8.05 (s, 1H), 6.44 (d, J=9.2 Hz, 1H), 6.22 (tt, J=55.6, 4.5 Hz, 1H), 4.66 (td, J=13.3, 4.5 Hz, 2H), 3.99-3.62 (m, 6H), 3.52 (t, J=7.1 Hz, 1H), 3.32 (s, 1H), 2.58 (s, 3H), 2.02 (dt, J=26.2, 7.1 Hz, 2H), 1.77 (dq, J=12.7, 6.4, 5.8 Hz, 4H). MS m/z: 483 [M+H]⁺.

### Example 512: 8-[1-(2,2-difluoroethyl)-1H-pyrazolo[3,4-b]pyrazin-6-yl]-2-[2-methyl-6-(trifluoromethyl)pyrimidin-4-yl]-2,8-diazaspiro[4.5]decan-1-one

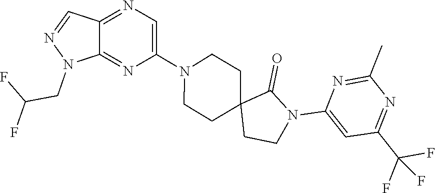 [SMILES: Cc1nc(N2CCC3(CCN(c4cnc5cnn(CC(F)F)c5n4)CC3)C2=O)cc(C(F)(F)F)n1]

Followed the General procedure A to afford the desired product as a yellow solid (11 mg, 25%). ¹H NMR (500 MHz, CDCl₃) δ 8.59 (s, 1H), 8.27 (s, 1H), 8.06 (s, 1H), 6.22 (tt, J=55.6, 4.5 Hz, 1H), 4.67 (td, J=13.3, 4.5 Hz, 1H), 4.33 (dt, J=13.8, 4.8 Hz, 2H), 4.16-4.07 (m, 2H), 3.54 (ddd, J=13.4, 9.8, 3.3 Hz, 2H), 2.22-2.15 (m, 2H), 2.11 (ddd, J=13.8, 9.8, 4.0 Hz, 2H), 1.78-1.68 (m, 2H). MS m/z: 497 [M+H]⁺.

### Example 513: 8-[1-(2,2-difluoroethyl)-1H-pyrazolo[3,4-b]pyrazin-6-yl]-2-[2-methyl-6-(trifluoromethyl)pyrimidin-4-yl]-2,8-diazaspiro[4.5]decan-3-one

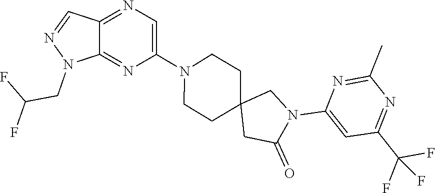 [SMILES: Cc1nc(N2CC3(CCN(c4cnc5cnn(CC(F)F)c5n4)CC3)CC2=O)cc(C(F)(F)F)n1]

Followed the General procedure A to afford the desired product as a yellow solid (32 mg, 73%). ¹H NMR (500 MHz, CDCl₃) δ 8.57 (s, 1H), 8.27 (s, 1H), 8.06 (s, 1H), 6.21 (tt, J=55.5, 4.5 Hz, 2H), 4.97-4.73 (m, 15H), 4.67 (td, J=13.3, 4.5 Hz, 3H), 4.02 (s, 2H), 3.94 (ddd, J=13.9, 6.6, 4.4 Hz, 2H), 3.74 (ddd, J=13.8, 7.4, 4.3 Hz, 2H), 2.72 (s, 3H), 2.71 (s, 2H), 1.86 (dt, J=6.6, 4.6 Hz, 4H). MS m/z: 497 [M+H]⁺.

### Example 514: 7-[1-(2,2-difluoroethyl)-3-methyl-1H-pyrazolo[3,4-b]pyrazin-6-yl]-2-[2-(trifluoromethyl)pyridin-4-yl]-2,7-diazaspiro[4.4]nonan-1-one

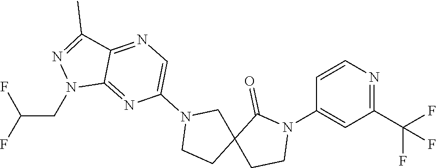 [SMILES: Cc1nn(CC(F)F)c2nc(N3CCC4(CCN(c5ccnc(C(F)(F)F)c5)C4=O)C3)cnc12]

Followed the General procedure A to afford the desired product as a white solid (23 mg, 64%). ¹H NMR (500 MHz, CDCl₃) δ 8.69 (d, J=5.6 Hz, 1H), 8.09 (d, J=2.1 Hz, 1H), 7.91 (s, 1H), 7.84 (dd, J=5.7, 2.2 Hz, 1H), 6.19 (tt, J=55.7, 4.5 Hz, 1H), 4.58 (tdd, J=13.5, 4.6, 2.2 Hz, 2H), 4.02-3.91 (m, 4H), 3.77 (dt, J=11.0, 8.0 Hz, 2H), 2.55 (s, 4H), 2.44-2.28 (m, 2H), 2.17 (ddd, J=12.2, 7.3, 4.5 Hz, 1H). MS m/z: 482 [M+H]⁺.

### Example 515: 7-[1-(2,2-difluoroethyl)-3-methyl-1H-pyrazolo[3,4-d]pyrimidin-6-yl]-2-[2-(trifluoromethyl)pyridin-4-yl]-2,7-diazaspiro[4.4]nonan-1-one

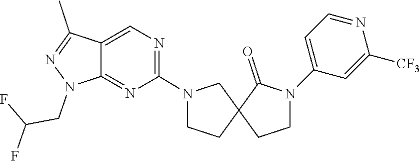 [SMILES: Cc1nn(CC(F)F)c2nc(N3CCC4(CCN(c5ccnc(C(F)(F)F)c5)C4=O)C3)ncc12]

Followed the General procedure A to afford the desired product as a white solid (22 mg, 64%). ¹H NMR (500 MHz, CDCl₃) δ 8.69 (d, J=5.6 Hz, 1H), 8.09 (d, J=2.1 Hz, 1H), 7.91 (s, 1H), 7.84 (dd, J=5.7, 2.2 Hz, 1H), 6.19 (tt, J=55.7, 4.5 Hz, 1H), 4.58 (tdd, J=13.5, 4.6, 2.2 Hz, 2H), 4.02-3.91 (m, 4H), 3.77 (dt, J=11.0, 8.0 Hz, 2H), 2.55 (s, 4H), 2.44-2.28 (m, 2H), 2.17 (ddd, J=12.2, 7.3, 4.5 Hz, 1H). MS m/z: 482 [M+H]⁺.

### Example 516: 7-[1-(2,2-difluoroethyl)-1H-pyrazolo[3,4-b]pyrazin-6-yl]-2-[2-methyl-6-(trifluoromethyl)pyrimidin-4-yl]-2,7-diazaspiro[4.4]nonan-1-one

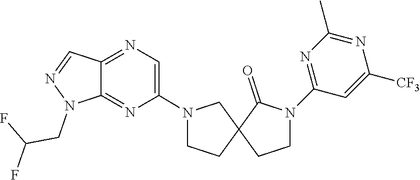 [SMILES: Cc1nc(N2CCC3(CCN(c4cnc5cnn(CC(F)F)c5n4)C3)C2=O)cc(C(F)(F)F)n1]

Followed the General procedure A to afford the desired product as a white solid (22 mg, 61%). ¹H NMR (500 MHz, CDCl₃) δ 8.58 (s, 1H), 8.06 (s, 1H), 7.99 (s, 1H), 6.22 (tt, J=55.6, 4.5 Hz, 1H), 4.66 (td, J=13.4, 4.5 Hz, 2H), 4.24-4.11 (m, 2H), 4.03-3.94 (m, 2H), 3.83-3.72 (m, 2H), 2.75 (s, 3H), 2.54 (dt, J=15.5, 7.8 Hz, 1H), 2.37-2.24 (m, 2H), 2.19 (ddd, J=12.5, 7.4, 5.0 Hz, 1H), 2.01 (s, 2H). MS m/z: 483 [M+H]⁺.

### Example 517: 7-[1-(2,2-difluoroethyl)-1H-pyrazolo[3,4-b]pyrazin-6-yl]-2-[2-(trifluoromethyl)pyridin-4-yl]-2,7-diazaspiro[4.4]nonan-1-one

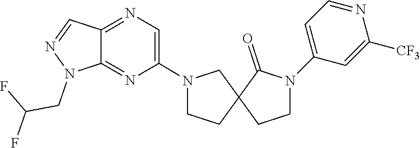 [SMILES: O=C1N(c2ccnc(C(F)(F)F)c2)CCC12CCN(c1cnc3cnn(CC(F)F)c3n1)C2]

Followed the General procedure A to afford the desired product as a white solid (24 mg, 68%). ¹H NMR (500 MHz, CDCl₃) δ 8.69 (d, J=5.6 Hz, 1H), 8.09 (d, J=2.2 Hz, 1H), 8.06 (s, 1H), 7.99 (s, 1H), 7.84 (dd, J=5.6, 2.1 Hz, 1H), 6.22 (tt, J=55.6, 4.5 Hz, 1H), 4.66 (tdd, J=13.5, 4.6, 2.1 Hz, 2H), 4.02-3.92 (m, 4H), 3.83-3.72 (m, 2H), 2.56 (dt, J=12.8, 8.1 Hz, 1H), 2.44-2.30 (m, 2H), 2.18 (ddd, J=12.3, 7.2, 4.6 Hz, 1H). MS m/z : 468 [M+H]⁺.

### Example 518: 2-[1-(2,2-difluoroethyl)-1H-pyrazolo[3,4-b]pyrazin-6-yl]-7-[2-(trifluoromethyl)pyridin-4-yl]-2,7-diazaspiro[4.4]nonan-1-one

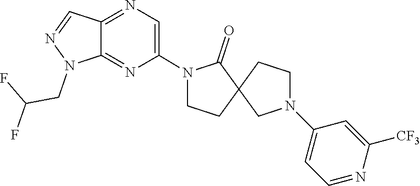 [SMILES: O=C1N(c2cnc3cnn(CC(F)F)c3n2)CCC12CCN(c1ccnc(C(F)(F)F)c1)C2]

Followed the General procedure A to afford the desired product as a white solid (12 mg, 37%). ¹H NMR (500 MHz, CDCl₃) δ 9.83 (s, 1H), 8.33 (d, J=5.8 Hz, 1H), 8.30 (s, 1H), 6.78 (d, J=2.4 Hz, 1H), 6.52 (dd, J=5.9, 2.4 Hz, 1H), 6.37-6.12 (m, 1H), 4.79 (td, J=13.3, 4.4 Hz, 2H), 4.30-4.14 (m, 2H), 3.83 (d, J=9.9 Hz, 1H), 3.74 (td, J=9.0, 4.3 Hz, 1H), 3.58 (dt, J=9.7, 7.6 Hz, 1H), 3.48 (d, J=10.0 Hz, 1H), 2.56 (dt, J=12.6, 8.1 Hz, 1H), 2.34 (t, J=7.0 Hz, 2H), 2.20 (ddd, J=12.1, 7.2, 4.2 Hz, 1H). MS m/z: 468 [M+H]⁺.

### Example 519: 2-[1-(2,2-difluoroethyl)-1H-pyrazolo[3,4-b]pyrazin-6-yl]-7-[6-(trifluoromethyl)pyridin-3-yl]-2,7-diazaspiro[4.4]nonan-1-one

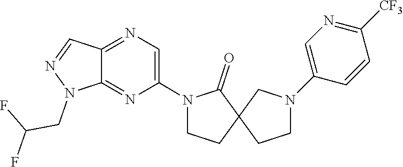 [SMILES: O=C1N(c2cnc3cnn(CC(F)F)c3n2)CCC12CCN(c1ccc(C(F)(F)F)nc1)C2]

Followed the General procedure A to afford the desired product as a white solid (5 mg, 19%). ¹H NMR (500 MHz, CDCl₃) δ 9.84 (s, 1H), 8.30 (s, 1H), 8.05 (d, J=2.9 Hz, 1H), 7.52 (d, J=8.7 Hz, 1H), 6.87 (dd, J=8.7, 2.8 Hz, 1H), 6.35-6.11 (m, 2H), 4.79 (td, J=13.3, 4.4 Hz, 2H), 4.21 (ddt, J=19.0, 11.8, 6.3 Hz, 2H), 3.83 (d, J=9.6 Hz, 1H), 3.72 (td, J=8.8, 4.0 Hz, 1H), 3.60 (q, J=8.1 Hz, 1H), 3.48 (d, J=9.6 Hz, 1H), 2.56 (dt, J=12.6, 8.2 Hz, 1H), 2.34 (t, J=6.9 Hz, 2H), 2.20 (ddd, J=12.0, 7.1, 4.1 Hz, 1H). MS m/z: 268 [M+H]⁺.

### Example 520: 2-[1-(2,2-difluoroethyl)-1H-pyrazolo[3,4-b]pyrazin-6-yl]-7-[5-(trifluoromethyl)pyridin-2-yl]-2,7-diazaspiro[4.4]nonan-1-one

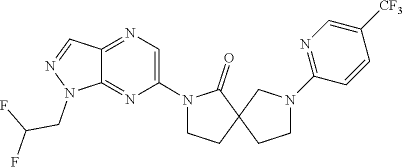 [SMILES: O=C1N(c2cnc3cnn(CC(F)F)c3n2)CCC12CCN(c1ccc(C(F)(F)F)cn1)C2]

Followed the General procedure A to afford the desired product as a white solid (11 mg, 34%). ¹H NMR (500 MHz, CDCl₃) δ 9.84 (d, J=0.9 Hz, 1H), 8.46-8.34 (m, 1H), 8.29 (d, J=1.0 Hz, 1H), 7.64 (dd, J=9.1, 2.5 Hz, 1H), 6.42 (d, J=8.9 Hz, 1H), 6.24 (tt, J=55.4, 4.4 Hz, 1H), 4.79 (td, J=13.3, 4.4 Hz, 2H), 4.19 (tt, J=8.9, 4.2 Hz, 2H), 3.98-3.83 (m, 2H), 3.66 (dq, J=17.4, 9.3, 8.5 Hz, 2H), 2.55 (dt, J=12.6, 8.1 Hz, 1H), 2.39-2.26 (m, 2H), 2.17 (ddd, J=12.2, 7.2, 4.4 Hz, 1H). MS m/z: 468 [M+H]⁺.

### Example 521: 2-[1-(2,2-difluoroethyl)-1H-pyrazolo[3,4-b]pyrazin-6-yl]-7-[2-methyl-6-(trifluoromethyl)pyrimidin-4-yl]-2,7-diazaspiro[4.4]nonane

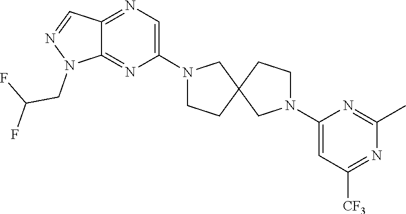 [SMILES: Cc1nc(N2CCC3(CCN(c4cnc5cnn(CC(F)F)c5n4)C3)C2)cc(C(F)(F)F)n1]

Followed the General procedure A to afford the desired product as a white solid (21 mg, 60%). ¹H NMR (500 MHz, CDCl₃) δ 8.05 (s, 1H), 7.96 (s, 1H), 6.45 (d, J=38.3 Hz, 1H), 6.22 (tt, J=55.6, 4.5 Hz, 1H), 4.66 (td, J=13.4, 4.5 Hz, 2H), 3.93-3.40 (m, 9H), 2.58 (d, J=16.3 Hz, 3H), 2.30-2.07 (m, 4H). MS m/z: 469 [M+H]⁺.

### Example 522: 7-[1-(2,2-difluoroethyl)-1H-pyrazolo[3,4-b]pyrazin-6-yl]-2-[2-methyl-6-(trifluoromethyl)pyrimidin-4-yl]-2,7-diazaspiro[3.5]nonane

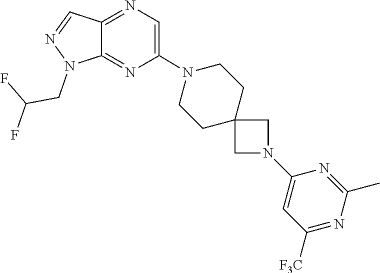 [SMILES: Cc1nc(N2CC3(CCN(c4cnc5cnn(CC(F)F)c5n4)CC3)C2)cc(C(F)(F)F)n1]

Followed the General procedure A to afford the desired product as a white solid (13 mg, 37%). ¹H NMR (500 MHz, CDCl₃) δ 8.26 (s, 1H), 8.05 (d, J=1.2 Hz, 1H), 6.35 (s, 1H), 6.34-6.08 (m, 1H), 4.67 (td, J=13.4, 4.5 Hz, 2H), 3.86 (dd, J=84.1, 51.4 Hz, 8H), 2.58 (s, 3H), 1.98 (t, J=5.5 Hz, 4H). MS m/z: 469 [M+H]⁺.

### Example 523: 2-[1-(2,2-difluoroethyl)-1H-pyrazolo[3,4-b]pyrazin-6-yl]-7-[2-methyl-6-(trifluoromethyl)pyrimidin-4-yl]-2,7-diazaspiro[4.4]nonan-1-one

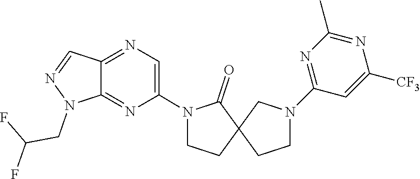 [SMILES: Cc1nc(N2CCC3(CCN(c4cnc5cnn(CC(F)F)c5n4)C3=O)C2)cc(C(F)(F)F)n1]

Followed the General procedure A to afford the desired product as a white solid (65 mg, 29.4%). ¹H NMR (500 MHz, CDCl₃) δ 9.82 (s, 1H), 8.30 (s, 1H), 6.53-6.44 (m, 1H), 6.24 (tt, J=55.4, 4.4 Hz, 1H), 4.79 (td, J=13.3, 4.4 Hz, 2H), 4.21 (s, 2H), 4.14-3.98 (m, 1H), 3.87 (d, J=11.8 Hz, 1H), 3.79 (s, 1H), 3.53 (dd, J=59.7, 9.5 Hz, 1H), 2.58 (d, J=12.6 Hz, 4H), 2.34 (t, J=6.7 Hz, 2H), 2.19 (d, J=14.7 Hz, 1H). MS m/z: 483 [M+H]⁺.

### Example 524: 6-[1-(2,2-difluoroethyl)-1H-pyrazolo[3,4-b]pyrazin-6-yl]-2-[2-methyl-6-(trifluoromethyl)pyrimidin-4-yl]-2,6-diazaspiro[3.4]octan-5-one

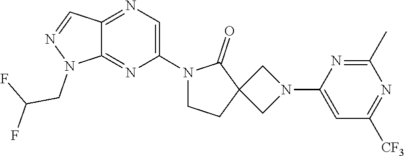 [SMILES: Cc1nc(N2CC3(CCN(c4cnc5cnn(CC(F)F)c5n4)C3=O)C2)cc(C(F)(F)F)n1]

Followed the General procedure A to afford the desired product as a white solid (28 mg, 87%). ¹H NMR (500 MHz, CDCl₃) δ 9.85 (d, J=1.4 Hz, 1H), 8.30 (d, J=1.4 Hz, 1H), 6.39 (s, 1H), 6.23 (tt, J=55.4, 4.5 Hz, 1H), 4.78 (td, J=13.3, 4.4 Hz, 2H), 4.59-4.47 (m, 2H), 4.19 (t, J=7.1 Hz, 4H), 2.65 (t, J=7.0 Hz, 2H), 2.59 (s, 3H). MS m/z: 469 [M+H]⁺.

### Example 525: 6-[1-(2,2-difluoroethyl)-1H-pyrazolo[3,4-b]pyrazin-6-yl]-2-[2-methyl-6-(trifluoromethyl)pyrimidin-4-yl]-2,6-diazaspiro[3.4]octan-7-one

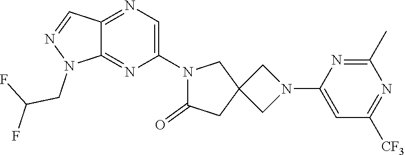 [SMILES: Cc1nc(N2CC3(CC(=O)N(c4cnc5cnn(CC(F)F)c5n4)C3)C2)cc(C(F)(F)F)n1]

Followed the General procedure A to afford the desired product as a white solid (24 mg, 72%). ¹H NMR (500 MHz, CDCl₃) δ 9.79 (s, 1H), 8.30 (s, 1H), 6.38 (s, 1H), 6.22 (tt, J=55.4, 4.4 Hz, 1H), 4.77 (td, J=13.3, 4.4 Hz, 2H), 4.44 (s, 2H), 4.35-4.20 (m, 4H), 3.11 (s, 2H), 2.59 (s, 3H). MS m/z: 469 [M+H]⁺.

### Example 526: 7-[1-(2,2-difluoroethyl)-1H-pyrazolo[3,4-b]pyrazin-6-yl]-2-[2-(trifluoromethyl)pyrimidin-5-yl]-2,7-diazaspiro[3.5]nonane

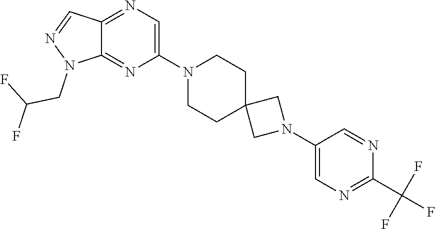 [SMILES: FC(F)Cn1ncc2ncc(N3CCC4(CC3)CN(c3cnc(C(F)(F)F)nc3)C4)nc21]

Followed the General procedure A to afford the desired product as a white solid (5 mg, 13%). ¹H NMR (500 MHz, CDCl₃) δ 8.27 (s, 1H), 8.06 (s, 1H), 8.01 (s, 2H), 6.23 (tt, J=55.4, 4.5 Hz, 1H), 4.67 (td, J=13.4, 4.5 Hz, 2H), 3.91 (s, 4H), 3.84-3.71 (m, 4H), 2.05-1.95 (m, 3H). MS m/z: 455 [M+H]⁺.

### Example 527: 7-[1-(2,2-difluoroethyl)-1H-pyrazolo[3,4-b]pyrazin-6-yl]-2-[6-methyl-2-(trifluoromethyl)pyrimidin-4-yl]-2,7-diazaspiro[3.5]nonane

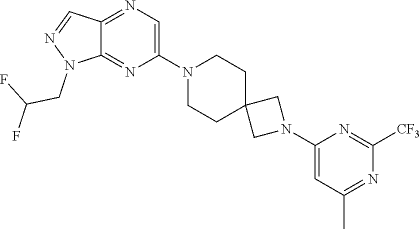 [SMILES: Cc1cc(N2CC3(CCN(c4cnc5cnn(CC(F)F)c5n4)CC3)C2)nc(C(F)(F)F)n1]

Followed the General procedure A to afford the desired product as a white solid (20 mg, 49%). ¹H NMR (500 MHz, CDCl₃) δ 8.26 (s, 1H), 8.05 (s, 1H), 6.21 (tt, J=55.6, 4.5 Hz, 1H), 6.14 (s, 1H), 4.67 (td, J=13.3, 4.5 Hz, 2H), 3.89 (s, 8H), 2.43 (s, 3H), 1.97 (t, J=5.5 Hz, 4H) MS m/z: 469 [M+H]⁺.

### Example 528: 8-[1-(2,2-difluoroethyl)-1H-pyrazolo[3,4-b]pyrazin-6-yl]-2-[6-methyl-2-(trifluoromethyl)pyrimidin-4-yl]-2,8-diazaspiro[4.5]decane

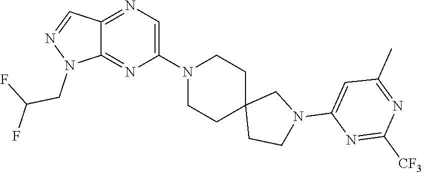 [SMILES: Cc1cc(N2CCC3(CCN(c4cnc5cnn(CC(F)F)c5n4)CC3)C2)nc(C(F)(F)F)n1]

Followed the General procedure A to afford the desired product as a off-white solid (20 mg, 72%). ¹H NMR (500 MHz, CDCl₃) δ 8.25 (s, 1H), 8.05 (s, 1H), 6.23 (s, 1H), 6.21 (tt, J=55.6, 4.5 Hz, 1H), 4.66 (td, J=13.3, 4.5 Hz, 2H), 4.00-3.61 (m, 6H), 3.49 (t, J=7.2 Hz, 1H), 3.29 (s, 1H), 2.44 (s, 3H), 2.01 (t, J=11.0 Hz, 2H), 1.85-1.67 (m, 4H). MS m/z: 483 [M+H]⁺.

### Example 529: 8-[1-(2,2-difluoroethyl)-1H-pyrazolo[3,4-b]pyrazin-6-yl]-2-[6-methyl-2-(trifluoromethyl)pyrimidin-4-yl]-2,8-diazaspiro[4.5]decan-3-one

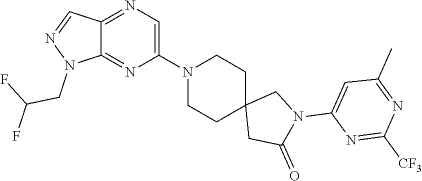 [SMILES: Cc1cc(N2CC3(CCN(c4cnc5cnn(CC(F)F)c5n4)CC3)CC2=O)nc(C(F)(F)F)n1]

Followed the General procedure A to afford the desired product as a white solid (27 mg, 78%). ¹H NMR (500 MHz, CDCl₃) δ 8.39 (s, 1H), 8.25 (s, 1H), 8.04 (s, 1H), 6.19 (tt, J=55.5, 4.5 Hz, 1H), 4.65 (td, J=13.3, 4.5 Hz, 2H), 4.00 (s, 2H), 3.94 (ddd, J=13.9, 6.8, 4.0 Hz, 2H), 3.70 (ddd, J=13.8, 7.9, 3.9 Hz, 2H), 2.69 (s, 2H), 2.61 (s, 3H), 1.84 (qdt, J=10.7, 6.9, 4.0 Hz, 4H). MS m/z: 497 [M+H]⁺.

### Example 530: 2-[1-(2,2-difluoroethyl)-3-methyl-1H-pyrazolo[3,4-b]pyrazin-6-yl]-7-[2-(trifluoromethyl)pyrimidin-5-yl]-2,7-diazaspiro[4.4]nonane

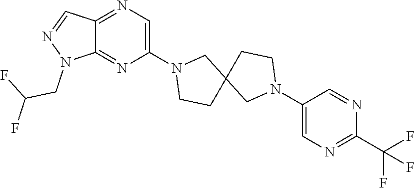 [SMILES: FC(F)Cn1ncc2ncc(N3CCC4(CCN(c5cnc(C(F)(F)F)nc5)C4)C3)nc21]

Followed the General procedure A to afford the desired product as a white solid (18.7 mg, 51%). ¹H NMR (500 MHz, CDCl₃) δ 8.12 (s, 2H), 7.89 (s, 1H), 6.20 (tt, J=55.7, 4.5 Hz, 1H), 4.59 (td, J=13.4, 4.5 Hz, 2H), 3.80 (q, J=7.1 Hz, 2H), 3.67 (d, J=5.4 Hz, 2H), 3.62 (t, J=7.0 Hz, 2H), 3.51-3.39 (m, 2H), 2.55 (s, 3H), 2.33-2.12 (m, 4H). MS m/z: 469 [M+H]⁺.

### Example 531: 2-[1-(2,2,2-trifluoroethyl)-1H-pyrazolo[3,4-b]pyrazin-6-yl]-7-[2-(trifluoromethyl)pyrimidin-5-yl]-2,7-diazaspiro[4.4]nonane

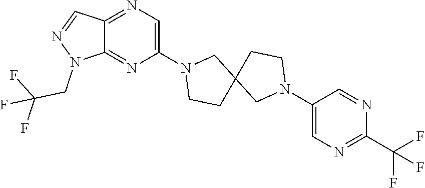 [SMILES: FC(F)(F)Cn1ncc2ncc(N3CCC4(CCN(c5cnc(C(F)(F)F)nc5)C4)C3)nc21]

Followed the General procedure A to afford the desired product as a white solid (18.9 mg, 52%). ¹H NMR (500 MHz, CDCl₃) δ 8.12 (s, 2H), 8.10 (s, 1H), 7.99 (s, 1H), 4.89 (q, J=8.4 Hz, 2H), 3.81 (q, J=7.4 Hz, 2H), 3.74-3.66 (m, 2H), 3.62 (t, J=6.9 Hz, 2H), 3.47 (q, J=9.6 Hz, 2H), 2.40-2.08 (m, 4H). MS m/z: 473 [M+H]⁺.

### Example 532: 2-[6-(1,3,4-thiadiazol-2-yl)pyrazin-2-yl]-7-[2-(trifluoromethyl)pyrimidin-5-yl]-2,7-diazaspiro[4.4]nonane

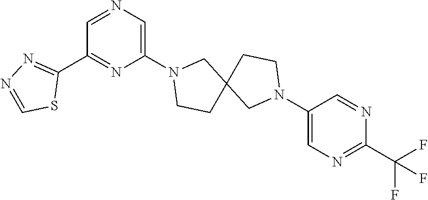 [SMILES: FC(F)(F)c1ncc(N2CCC3(CCN(c4cncc(-c5nncs5)n4)C3)C2)cn1]

Followed the General procedure A to afford the desired product as a white solid (15.6 mg, 46%). ¹H NMR (500 MHz, CDCl₃) δ 9.19 (s, 1H), 8.87 (s, 1H), 8.12 (s, 2H), 8.01 (s, 1H), 3.76 (q, J=6.6 Hz, 2H), 3.68-3.56 (m, 4H), 3.56-3.37 (m, 2H), 2.36-2.10 (m, 4H). MS m/z: 435 [M+H]⁺.

### Example 533: 2-[1-(oxetan-3-yl)-1H-pyrazolo[3,4-b]pyrazin-6-yl]-7-[2-(trifluoromethyl)pyrimidin-5-yl]-2,7-diazaspiro[4.4]nonane

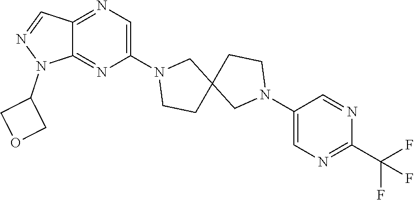 [SMILES: FC(F)(F)c1ncc(N2CCC3(CCN(c4cnc5cnn(C6COC6)c5n4)C3)C2)cn1]

Followed the General procedure A to afford the desired product as a white solid (21.5 mg, 62%). ¹H NMR (500 MHz, CDCl₃) δ 8.12 (s, 2H), 8.10 (s, 1H), 7.96 (s, 1H), 5.98-5.90 (m, 1H), 5.34 (t, J=6.6 Hz, 2H), 5.08-5.01 (m, 2H), 3.80 (q, J=7.2 Hz, 2H), 3.73-3.59 (m, 4H), 3.46 (q, J=9.6 Hz, 2H), 2.28-2.16 (m, 4H). MS m/z: 447 [M+H]⁺.

### Example 534: 2-[3-methyl-1-(oxetan-3-yl)-1H-pyrazolo[3,4-b]pyrazin-6-yl]-7-[2-(trifluoromethyl)pyrimidin-5-yl]-2,7-diazaspiro[4.4]nonane

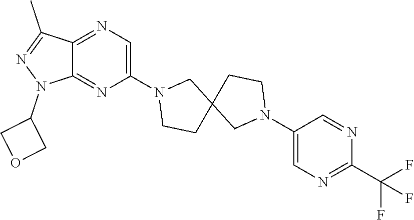 [SMILES: Cc1nn(C2COC2)c2nc(N3CCC4(CCN(c5cnc(C(F)(F)F)nc5)C4)C3)cnc12]

Followed the General procedure A to afford the desired product as a white solid (18.9 mg, 19%). ¹H NMR (500 MHz, CDCl₃) δ 8.11 (s, 2H), 7.88 (s, 1H), 5.87 (tt, J=7.9, 6.8 Hz, 1H), 5.32 (t, J=6.6 Hz, 2H), 5.02 (ddd, J=8.4, 6.5, 2.2 Hz, 2H), 3.96-3.70 (m, 2H), 3.70-3.54 (m, 4H), 3.45 (q, J=9.6 Hz, 2H), 2.59 (s, 3H), 2.33-2.08 (m, 4H). MS m/z: 461 [M+H]⁺.

### Example 535: 2-[1-(oxetan-3-yl)-1H-pyrazolo[3,4-d]pyrimidin-6-yl]-7-[2-(trifluoromethyl)pyrimidin-5-yl]-2,7-diazaspiro[4.4]nonane

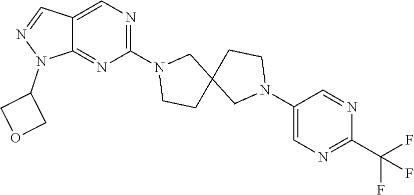 [SMILES: FC(F)(F)c1ncc(N2CCC3(CCN(c4ncc5cnn(C6COC6)c5n4)C3)C2)cn1]

Followed the General procedure A to afford the desired product as a white foam (9 mg, 26%). ¹H NMR (500 MHz, CDCl₃) δ 8.77 (s, 1H), 8.10 (s, 2H), 7.95 (s, 1H), 6.05-5.87 (m, 1H), 5.30 (t, J=6.6 Hz, 2H), 5.05 (t, J=7.3 Hz, 2H), 3.76 (d, J=74.2 Hz, 4H), 3.59 (t, J=7.0 Hz, 2H), 3.43 (q, J=9.6 Hz, 2H), 2.35-1.99 (m, 4H). MS m/z: 447 [M+H]⁺.

### Example 536: 2-[3-methyl-1-(oxetan-3-yl)-1H-pyrazolo[3,4-d]pyrimidin-6-yl]-7-[2-(trifluoromethyl)pyrimidin-5-yl]-2,7-diazaspiro[4.4]nonane

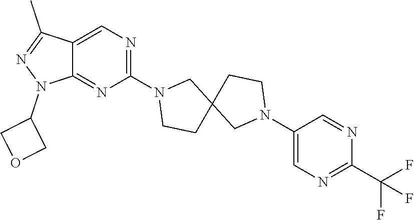 [SMILES: Cc1nn(C2COC2)c2nc(N3CCC4(CCN(c5cnc(C(F)(F)F)nc5)C4)C3)ncc12]

Followed the General procedure A to afford the desired product as a white solid (18 mg, 50%). ¹H NMR (500 MHz, CDCl₃) δ 8.66 (s, 1H), 8.09 (s, 2H), 5.87 (t, J=7.3 Hz, 1H), 5.28 (t, J=6.5 Hz, 2H), 5.14-4.89 (m, 2H), 3.75 (d, J=74.8 Hz, 4H), 3.58 (t, J=6.9 Hz, 2H), 3.42 (q, J=9.6 Hz, 2H), 2.53 (s, 3H), 2.33-1.92 (m, 4H). MS m/z: 461 [M+H]⁺.

### Example 537: 2-[1-(2,2-difluoroethyl)-3-methyl-1H-pyrazolo[3,4-b]pyrazin-6-yl]-7-[2-methyl-6-(trifluoromethyl)pyrimidin-4-yl]-2,7-diazaspiro[4.4]nonane

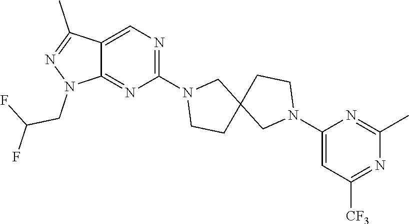 [SMILES: Cc1nc(N2CCC3(CCN(c4ncc5c(C)nn(CC(F)F)c5n4)C3)C2)cc(C(F)(F)F)n1]

Followed the General procedure A to afford the desired product as a white solid (16.4 mg, 45%). ¹H NMR (500 MHz, CDCl₃) δ 7.88 (s, 1H), 6.45 (d, J=38.1 Hz, 1H), 6.20 (tt, J=55.7, 4.5 Hz, 1H), 4.58 (td, J=13.4, 4.5 Hz, 2H), 3.97-3.36 (m, 8H), 2.60-2.55 (m, 2H), 2.55 (s, 3H), 2.27-2.07 (m, 4H). MS m/z: 483 [M+H]⁺.

### Example 538: 2-[2-methyl-6-(trifluoromethyl)pyrimidin-4-yl]-7-[1-(2,2,2-trifluoroethyl)-1H-pyrazolo[3,4-b]pyrazin-6-yl]-2,7-diazaspiro[4.4]nonane

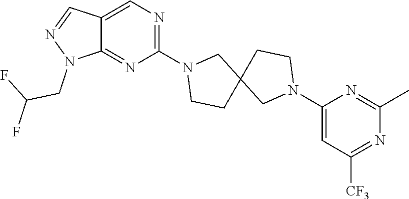 [SMILES: Cc1nc(N2CCC3(CCN(c4ncc5cnn(CC(F)F)c5n4)C3)C2)cc(C(F)(F)F)n1]

Followed the General procedure A to afford the desired product as a white solid (17 mg, 47%). ¹H NMR (500 MHz, CDCl₃) δ 8.09 (s, 1H), 7.97 (s, 1H), 6.45 (d, J=37.2 Hz, 1H), 5.01-4.72 (m, 2H), 4.03-3.30 (m, 8H), 2.58 (d, J=16.4 Hz, 3H), 2.36-1.97 (m, 4H). MS m/z: 487 [M+H]⁺.

### Example 539: 2-[2-methyl-6-(trifluoromethyl)pyrimidin-4-yl]-7-[6-(1,3,4-thiadiazol-2-yl)pyrazin-2-yl]-2,7-diazaspiro[4.4]nonane

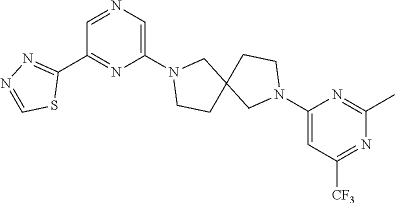 [SMILES: Cc1nc(N2CCC3(CCN(c4cncc(-c5nncs5)n4)C3)C2)cc(C(F)(F)F)n1]

Followed the General procedure A to afford the desired product as a white solid (14.4 mg, 45%). ¹H NMR (500 MHz, CDCl₃) δ 9.17 (s, 1H), 8.84 (s, 1H), 7.98 (s, 1H), 6.43 (d, J=35.0 Hz, 1H), 3.90-3.37 (m, 8H), 2.56 (d, J=15.9 Hz, 3H), 2.14 (d, J=19.2 Hz, 4H). MS m/z: 449 [M+H]⁺.

### Example 540: 2-[1-(2,2-difluoroethyl)-1H-pyrazolo[3,4-d]pyrimidin-6-yl]-7-[2-methyl-6-(trifluoromethyl)pyrimidin-4-yl]-2,7-diazaspiro[4.4]nonane

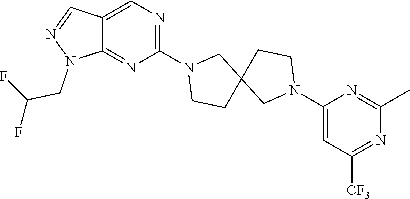 [SMILES: Cc1nc(N2CCC3(CCN(c4ncc5cnn(CC(F)F)c5n4)C3)C2)cc(C(F)(F)F)n1]

Followed the General procedure A to afford the desired product as a white solid (10 mg, 29%). ¹H NMR (500 MHz, CDCl₃) δ 8.77 (s, 1H), 7.90 (s, 1H), 6.44 (d, J=32.2 Hz, 1H), 6.22 (t, J=55.6 Hz, 1H), 4.63 (s, 2H), 3.93-3.37 (m, 8H), 2.57 (d, J=15.2 Hz, 3H), 2.26-2.02 (m, 4H). MS m/z: 469 [M+H]⁺.

### Example 541: 2-[1-(2,2-difluoroethyl)-3-methyl-1H-pyrazolo[3,4-d]pyrimidin-6-yl]-7-[2-methyl-6-(trifluoromethyl)pyrimidin-4-yl]-2,7-diazaspiro[4.4]nonane

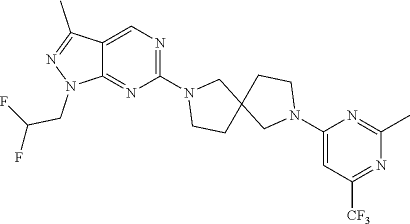 [SMILES: Cc1nc(N2CCC3(CCN(c4ncc5c(C)nn(CC(F)F)c5n4)C3)C2)cc(C(F)(F)F)n1]

Followed the General procedure A to afford the desired product as a white solid (14 mg, 40%). ¹H NMR (500 MHz, CDCl₃) δ 8.67 (s, 1H), 6.44 (d, J=32.1 Hz, 1H), 6.19 (t, J=55.6 Hz, 1H), 4.55 (s, 2H), 3.88-3.38 (m, 8H), 2.57 (d, J=14.9 Hz, 3H), 2.49 (s, 3H), 2.12 (q, J=14.8 Hz, 4H). MS m/z: 483 [M+H]⁺.

### Example 542: 2-[2-methyl-6-(trifluoromethyl)pyrimidin-4-yl]-7-[1-(oxetan-3-yl)-1H-pyrazolo[3,4-b]pyrazin-6-yl]-2,7-diazaspiro[4.4]nonane

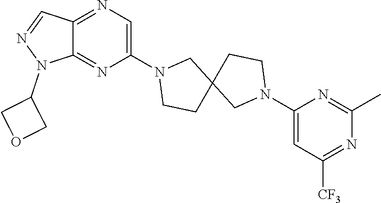 [SMILES: Cc1nc(N2CCC3(CCN(c4cnc5cnn(C6COC6)c5n4)C3)C2)cc(C(F)(F)F)n1]

Followed the General procedure A to afford the desired product as a off-white solid (16.2 mg, 49%). ¹H NMR (500 MHz, CDCl₃) δ 8.08 (s, 1H), 7.93 (s, 1H), 6.43 (d, J=41.3 Hz, 1H), 5.92 (p, J=7.3 Hz, 1H), 5.31 (td, J=6.6, 1.8 Hz, 2H), 5.03 (t, J=7.2 Hz, 2H), 3.96-3.33 (m, 8H), 2.56 (d, J=18.1 Hz, 3H), 2.25-1.96 (m, 4H). MS m/z: 461 [M+H]⁺.

### Example 543: 2-[3-methyl-1-(oxetan-3-yl)-1H-pyrazolo[3,4-b]pyrazin-6-yl]-7-[2-methyl-6-(trifluoromethyl)pyrimidin-4-yl]-2,7-diazaspiro[4.4]nonane

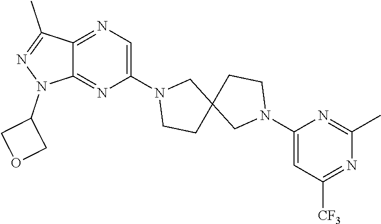 [SMILES: Cc1nc(N2CCC3(CCN(c4cnc5c(C)nn(C6COC6)c5n4)C3)C2)cc(C(F)(F)F)n1]

Followed the General procedure A to afford the desired product as a white solid (14.7 mg, 43%). ¹H NMR (500 MHz, CDCl₃) δ 7.86 (s, 1H), 6.45 (d, J=41.1 Hz, 1H), 5.98-5.81 (m, 1H), 5.32 (td, J=6.6, 1.6 Hz, 2H), 5.02 (t, J=7.2 Hz, 2H), 3.97-3.38 (m, 8H), 2.59 (s, 3H), 2.57 (M, 2H), 2.15 (m, 4H). MS m/z: 475 [M+H]⁺.

### Example 544: 2-[3-methyl-1-(oxetan-3-yl)-1H-pyrazolo[3,4-d]pyrimidin-6-yl]-7-[2-methyl-6-(trifluoromethyl)pyrimidin-4-yl]-2,7-diazaspiro[4.4]nonane

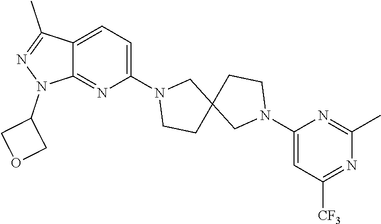 [SMILES: Cc1nc(N2CCC3(CCN(c4ccc5c(C)nn(C6COC6)c5n4)C3)C2)cc(C(F)(F)F)n1]

Followed the General procedure A to afford the desired product as a white solid (9 mg, 26%). ¹H NMR (500 MHz, CDCl₃) δ 8.63 (s, 1H), 6.41 (d, J=36.3 Hz, 2H), 5.95-5.79 (m, 1H), 5.26 (s, 2H), 5.00 (t, J=7.1 Hz, 2H), 3.90-3.38 (m, 8H), 2.69-2.41 (m, 2H), 2.40 (s, 3H), 2.11 (m, 4H). MS m/z: 475 [M+H]⁺.

### Example 545: 2-[2-methyl-6-(trifluoromethyl)pyrimidin-4-yl]-7-[1-(oxetan-3-yl)-1H-pyrazolo[3,4-d]pyrimidin-6-yl]-2,7-diazaspiro[4.4]nonane

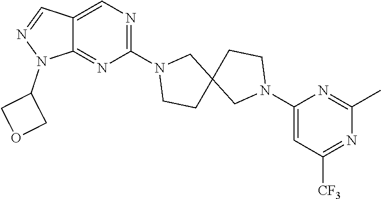 [SMILES: Cc1nc(N2CCC3(CCN(c4ncc5cnn(C6COC6)c5n4)C3)C2)cc(C(F)(F)F)n1]

Followed the General procedure A to afford the desired product as a white solid (12.2 mg, 37%). ¹H NMR (500 MHz, CDCl₃) δ 8.76 (s, 1H), 6.43 (d, J=36.3 Hz, 1H), 6.04-5.88 (m, 1H), 5.30 (s, 2H), 5.14-4.95 (m, 2H), 3.88-3.37 (m, 8H), 2.57 (d, J=17.4 Hz, 3H), 2.16 (d, J=38.3 Hz, 4H). MS m/z: 461 [M+H]⁺.

### Example 546: 2-[1-(2,2-difluoroethyl)-1H-pyrazolo[3,4-d]pyrimidin-6-yl]-7-[2-(trifluoromethyl)pyrimidin-5-yl]-2,7-diazaspiro[4.4]nonane

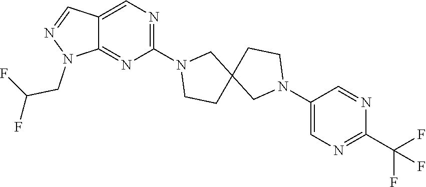 [SMILES: FC(F)Cn1ncc2cnc(N3CCC4(CCN(c5cnc(C(F)(F)F)nc5)C4)C3)nc21]

Followed the General procedure A to afford the desired product as a white solid (10.2 mg, 29%). ¹H NMR (500 MHz, CDCl₃) δ 8.78 (s, 1H), 8.10 (s, 2H), 7.90 (s, 1H), 6.22 (t, J=55.6 Hz, 1H), 4.64 (s, 2H), 4.06-3.52 (m, 6H), 3.52-3.26 (m, 2H), 2.37-2.05 (m, 4H). MS m/z: 455 [M+H]⁺.

### Example 547: 2-[1-(2,2-difluoroethyl)-3-methyl-1H-pyrazolo[3,4-d]pyrimidin-6-yl]-7-[2-(trifluoromethyl)pyrimidin-5-yl]-2,7-diazaspiro[4.4]nonane

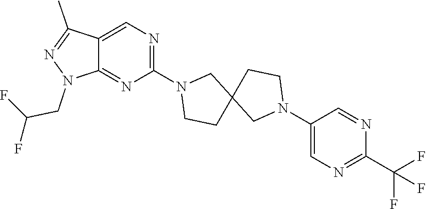 [SMILES: Cc1nn(CC(F)F)c2nc(N3CCC4(CCN(c5cnc(C(F)(F)F)nc5)C4)C3)ncc12]

Followed the General procedure A to afford the desired product as a white solid (27.8 mg, 76%). ¹H NMR (500 MHz, CDCl₃) δ 8.67 (s, 1H), 8.10 (s, 2H), 6.19 (t, J=55.7 Hz, 1H), 4.55 (t, J=12.6 Hz, 2H), 3.97-3.37 (m, 8H), 2.49 (s, 3H), 2.32-1.98 (m, 4H). MS m/z: 469 [M+H]⁺.

### Example 548: 2-[2-methyl-6-(trifluoromethyl)pyrimidin-4-yl]-7-[1-(2,2,2-trifluoroethyl)-1H-pyrazolo[3,4-d]pyrimidin-6-yl]-2,7-diazaspiro[4.4]nonane

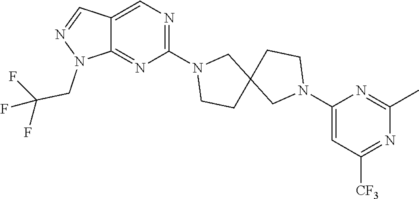 [SMILES: Cc1nc(N2CCC3(CCN(c4ncc5cnn(CC(F)(F)F)c5n4)C3)C2)cc(C(F)(F)F)n1]

Followed the General procedure A to afford the desired product as a white solid (14 mg, 40%). ¹H NMR (500 MHz, CDCl₃) δ 8.78 (d, J=14.1 Hz, 1H), 7.94 (s, 1H), 6.44 (d, J=31.0 Hz, 1H), 4.86 (s, 2H), 3.99-3.36 (m, 8H), 2.57 (d, J=15.0 Hz, 3H), 2.35-1.94 (m, 4H). MS m/z: 487 [M+H]⁺.

### Example 549: 8-[3-methyl-1-(oxetan-3-yl)-1H-pyrazolo[3,4-b]pyrazin-6-yl]-2-[2-methyl-6-(trifluoromethyl)pyrimidin-4-yl]-2,8-diazaspiro[4.5]decane

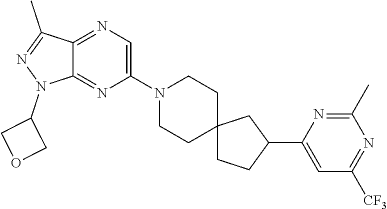 [SMILES: Cc1nc(C2CCC3(CCN(c4cnc5c(C)nn(C6COC6)c5n4)CC3)C2)cc(C(F)(F)F)n1]

Followed the General procedure A to afford the desired product as a white solid (16 mg, 46%). ¹H NMR (500 MHz, CDCl₃) δ 8.16 (s, 1H), 6.44 (d, J=10.8 Hz, 1H), 5.87 (p, J=7.3 Hz, 1H), 5.32 (t, J=6.6 Hz, 2H), 5.03 (dd, J=7.9, 6.4 Hz, 2H), 3.98-3.22 (m, 8H), 2.59 (s, 3H), 2.57 (s, 3H), 2.14-1.92 (m, 2H), 1.78 (dd, J=12.9, 7.1 Hz, 4H). MS m/z: 489 [M+H]⁺.

### Example 550: 8-[3-methyl-1-(oxetan-3-yl)-1H-pyrazolo[3,4-d]pyrimidin-6-yl]-2-[2-methyl-6-(trifluoromethyl)pyrimidin-4-yl]-2,8-diazaspiro[4.5]decane

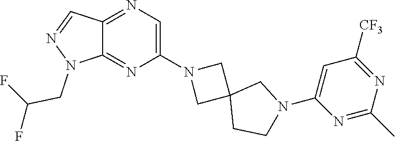 [SMILES: Cc1nc(N2CCC3(C2)CN(c2cnc4cnn(CC(F)F)c4n2)C3)cc(C(F)(F)F)n1]

Followed the General procedure A to afford the desired product as a white solid (16 mg, 45%). ¹H NMR (500 MHz, CDCl₃) δ 8.65 (s, 1H), 6.44 (d, J=7.6 Hz, 1H), 5.86 (p, J=7.3 Hz, 1H), 5.27 (t, J=6.5 Hz, 2H), 5.02 (t, J=7.2 Hz, 2H), 4.20-3.73 (m, 5H), 3.63 (s, 1H), 3.59-3.23 (m, 2H), 2.57 (s, 3H), 2.52 (s, 3H), 2.09-1.91 (m, 2H), 1.69 (dq, J=12.7, 6.8 Hz, 4H). MS m/z: 489 [M+H]⁺.

### Example 551: 2-[1-(2,2-difluoroethyl)-1H-pyrazolo[3,4-b]pyrazin-6-yl]-6-[2-methyl-6-(trifluoromethyl)pyrimidin-4-yl]-2,6-diazaspiro[3.4]octane

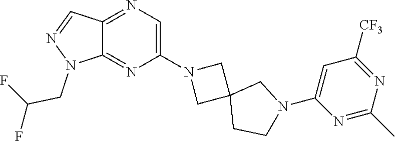 [SMILES: Cc1nc(N2CCC3(C2)CN(c2cnc4cnn(CC(F)F)c4n2)C3)cc(C(F)(F)F)n1]

Followed the General procedure A to afford the desired product as a white solid (16 mg, 45%). ¹H NMR (500 MHz, CDCl₃) δ 8.07 (s, 1H), 7.81 (s, 1H), 6.47 (s, 1H), 6.21 (tt, J=55.6, 4.5 Hz, 1H), 4.66 (td, J=13.3, 4.5 Hz, 2H), 4.22 (q, J=9.0 Hz, 4H), 4.08-3.40 (m, 4H), 2.58 (s, 3H), 2.38 (d, J=41.9 Hz, 2H). MS m/z: 455 [M+H]⁺.

### Example 552: 6-[2-methyl-6-(trifluoromethyl)pyrimidin-4-yl]-2-[1-(2,2,2-trifluoroethyl)-1H-pyrazolo[3,4-b]pyrazin-6-yl]-2,6-diazaspiro[3.4]octane

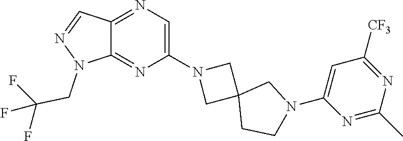 [SMILES: Cc1nc(N2CCC3(C2)CN(c2cnc4cnn(CC(F)(F)F)c4n2)C3)cc(C(F)(F)F)n1]

Followed the General procedure A to afford the desired product as a white solid (15 mg, 41%). ¹H NMR (500 MHz, CDCl₃) δ 8.11 (s, 1H), 7.82 (s, 1H), 6.46 (s, 1H), 4.88 (q, J=8.4 Hz, 2H), 4.29-4.18 (m, 4H), 4.05-3.44 (m, 4H), 2.58 (s, 3H), 2.50-2.24 (m, 2H). MS m/z: 473 [M+H]⁺.

### Example 553: 2-[1-(2,2-difluoroethyl)-1H-pyrazolo[3,4-d]pyrimidin-6-yl]-6-[2-methyl-6-(trifluoromethyl)pyrimidin-4-yl]-2,6-diazaspiro[3.4]octane

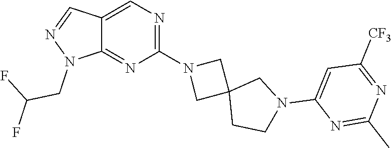 [SMILES: Cc1nc(N2CCC3(C2)CN(c2ncc4cnn(CC(F)F)c4n2)C3)cc(C(F)(F)F)n1]

Followed the General procedure A to afford the desired product as a white solid (12 mg, 34%). ¹H NMR (500 MHz, CDCl₃) δ 8.78 (s, 1H), 7.92 (s, 1H), 6.45 (d, J=10.8 Hz, 1H), 6.21 (tt, J=55.6, 4.5 Hz, 1H), 4.63 (td, J=13.3, 4.5 Hz, 2H), 4.19 (d, J=15.1 Hz, 4H), 4.03-3.47 (m, 4H), 2.58 (d, J=5.4 Hz, 3H), 2.47-2.22 (m, 2H). MS m/z: 455 [M+H]⁺.

### Example 554: 6-[2-methyl-6-(trifluoromethyl)pyrimidin-4-yl]-2-[1-(2,2,2-trifluoroethyl)-1H-pyrazolo[3,4-d]pyrimidin-6-yl]-2,6-diazaspiro[3.4]octane

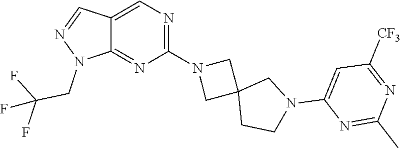 [SMILES: Cc1nc(N2CCC3(C2)CN(c2ncc4cnn(CC(F)(F)F)c4n2)C3)cc(C(F)(F)F)n1]

Followed the General procedure A to afford the desired product as a white solid (12 mg, 33%). ¹H NMR (500 MHz, CDCl₃) δ 8.79 (s, 1H), 7.96 (s, 1H), 6.46 (d, J=8.6 Hz, 1H), 4.86 (q, J=8.4 Hz, 2H), 4.30-3.43 (m, 8H), 2.58 (d, J=5.5 Hz, 3H), 2.51-2.21 (m, 2H). MS m/z: 473 [M+H]⁺.

### Example 555: 6-[2-methyl-6-(trifluoromethyl)pyrimidin-4-yl]-2-[1-(oxetan-3-yl)-1H-pyrazolo[3,4-b]pyrazin-6-yl]-2,6-diazaspiro[3.4]octane

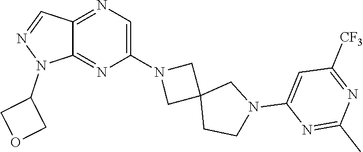 [SMILES: Cc1nc(N2CCC3(C2)CN(c2cnc4cnn(C5COC5)c4n2)C3)cc(C(F)(F)F)n1]

Followed the General procedure A to afford the desired product as a white solid (18 mg, 52%). ¹H NMR (500 MHz, CDCl₃) δ 8.12 (s, 1H), 7.80 (s, 1H), 6.46 (d, J=11.3 Hz, 1H), 5.93 (p, J=7.3 Hz, 1H), 5.31 (t, J=6.6 Hz, 2H), 5.05 (t, J=7.2 Hz, 2H), 4.33-3.46 (m, 9H), 2.57 (s, 3H), 2.48-2.23 (m, 2H). MS m/z: 447 [M+H]⁺.

### Example 556: 2-[3-methyl-1-(oxetan-3-yl)-1H-pyrazolo[3,4-b]pyrazin-6-yl]-6-[2-methyl-6-(trifluoromethyl)pyrimidin-4-yl]-2,6-diazaspiro[3.4]octane

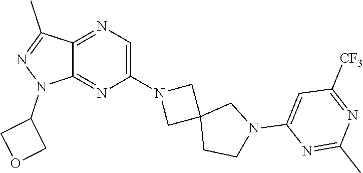 [SMILES: Cc1nc(N2CCC3(C2)CN(c2cnc4c(C)nn(C5COC5)c4n2)C3)cc(C(F)(F)F)n1]

Followed the General procedure A to afford the desired product as a white solid (13 mg, 36%). ¹H NMR (500 MHz, CDCl₃) δ 7.73 (s, 1H), 6.46 (s, 1H), 5.87 (p, J=7.3 Hz, 1H), 5.30 (t, J=6.6 Hz, 2H), 5.02 (t, J=7.2 Hz, 2H), 4.27-3.46 (m, 8H), 2.59 (s, 3H), 2.57 (d, J=10.8 Hz, 2H), 2.37 (m, 2H). MS m/z: 461 [M+H]⁺.

### Example 557: 6-[2-methyl-6-(trifluoromethyl)pyrimidin-4-yl]-2-[1-(oxetan-3-yl)-1H-pyrazolo[3,4-d]pyrimidin-6-yl]-2,6-diazaspiro[3.4]octane

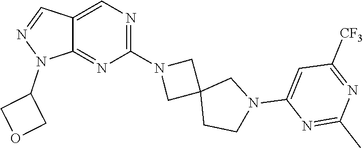 [SMILES: Cc1nc(N2CCC3(C2)CN(c2ncc4cnn(C5COC5)c4n2)C3)cc(C(F)(F)F)n1]

Followed the General procedure A to afford the desired product as a white solid (13 mg, 38%). ¹H NMR (500 MHz, CDCl₃) δ 8.78 (s, 1H), 7.98 (s, 1H), 6.45 (d, J=14.8 Hz, 1H), 6.05-5.77 (m, 1H), 5.27 (t, J=6.6 Hz, 2H), 5.05 (dd, J=7.8, 6.6 Hz, 2H), 4.34-3.50 (m, 8H), 2.58 (s, 3H), 2.44-2.14 (m, 2H). MS m/z: 447 [M+H]⁺.

### Example 558: 2-[3-methyl-1-(oxetan-3-yl)-1H-pyrazolo[3,4-d]pyrimidin-6-yl]-6-[2-methyl-6-(trifluoromethyl)pyrimidin-4-yl]-2,6-diazaspiro[3.4]octane

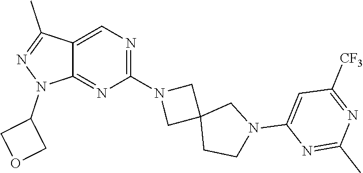 [SMILES: Cc1nc(N2CCC3(C2)CN(c2ncc4c(C)nn(C5COC5)c4n2)C3)cc(C(F)(F)F)n1]

Followed the General procedure A to afford the desired product as a white solid (15 mg, 42%). ¹H NMR (500 MHz, CDCl₃) δ 8.67 (s, 1H), 6.45 (d, J=15.7 Hz, 1H), 5.86 (p, J=7.2 Hz, 1H), 5.25 (t, J=6.5 Hz, 2H), 5.02 (t, J=7.2 Hz, 2H), 4.30-3.48 (m, 8H), 2.58 (d, J=7.0 Hz, 2H), 2.54 (s, 3H), 2.46-2.18 (m, 2H). MS m/z: 461 [M+H]⁺.

### Example 559: 6-[1-(2,2-difluoroethyl)-1H-pyrazolo[3,4-b]pyrazin-6-yl]-2-[2-(propan-2-yl)pyrimidin-5-yl]-2,6-diazaspiro[3.4]octane

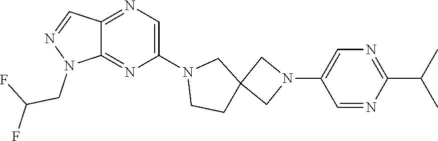 [SMILES: CC(C)c1ncc(N2CC3(CCN(c4cnc5cnn(CC(F)F)c5n4)C3)C2)cn1]

Followed the General procedure A to afford the desired product as a white solid (12 mg, 21%). ¹H NMR (500 MHz, CDCl₃) δ 8.05 (s, 1H), 7.98 (s, 2H), 7.96 (s, 1H), 6.22 (tt, J=55.7, 4.5 Hz, 1H), 4.66 (td, J=13.3, 4.5 Hz, 2H), 3.99 (q, J=7.2 Hz, 4H), 3.88 (s, 2H), 3.73 (t, J=6.9 Hz, 2H), 3.14 (p, J=6.9 Hz, 1H), 2.39 (t, J=6.9 Hz, 2H), 1.31 (s, 3H), 1.30 (s, 3H). MS m/z: 415 [M+H]⁺.

### Example 560: 2-(2-cyclopropylpyrimidin-5-yl)-6-[1-(2,2-difluoroethyl)-1H-pyrazolo[3,4-b]pyrazin-6-yl]-2,6-diazaspiro[3.4]octane

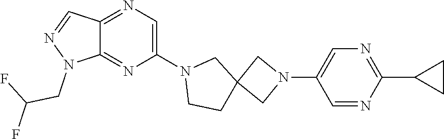 [SMILES: FC(F)Cn1ncc2ncc(N3CCC4(CN(c5cnc(C6CC6)nc5)C4)C3)nc21]

Followed the General procedure A to afford the desired product as a white solid (10 mg, 18%). ¹H NMR (500 MHz, CDCl₃) δ 8.05 (s, 1H), 7.96 (s, 1H), 7.89 (s, 2H), 6.22 (tt, J=55.6, 4.5 Hz, 1H), 4.66 (td, J=13.3, 4.5 Hz, 2H), 3.96 (q, J=7.2 Hz, 4H), 3.87 (s, 2H), 3.73 (t, J=6.9 Hz, 2H), 2.39 (t, J=6.9 Hz, 2H), 2.16 (tt, J=7.9, 5.2 Hz, 1H), 1.04-0.94 (m, 4H). MS m/z: 413 [M+H]⁺.

### Example 561: 7-[1-(2,2-difluoroethyl)-1H-pyrazolo[3,4-b]pyrazin-6-yl]-2-[2-methyl-4-(trifluoromethyl)pyrimidin-5-yl]-2,7-diazaspiro[3.5]nonane

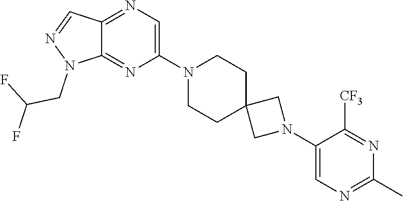 [SMILES: Cc1ncc(N2CC3(CCN(c4cnc5cnn(CC(F)F)c5n4)CC3)C2)c(C(F)(F)F)n1]

Followed the General procedure A to afford the desired product as a white solid (4.6 mg, 11%). ¹H NMR (500 MHz, CDCl₃) δ 8.26 (s, 1H), 8.09 (s, 1H), 8.05 (s, 1H), 6.34-6.13 (m, 1H), 4.67 (td, J=13.3, 4.5 Hz, 2H), 3.93 (s, 4H), 3.78 (t, J=5.7 Hz, 4H), 2.67 (s, 3H), 2.03-1.93 (m, 4H). MS m/z: 469 [M+H]⁺.

### Example 562: 2-[2-methyl-6-(trifluoromethyl)pyrimidin-4-yl]-7-[1-(oxetan-3-yl)-1H-pyrazolo[3,4-d]pyrimidin-6-yl]-2,7-diazaspiro[3.5]nonane

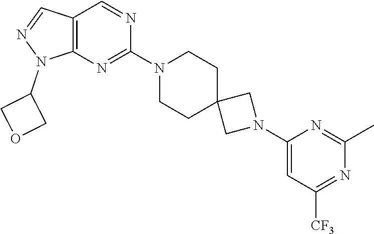 [SMILES: Cc1nc(N2CC3(CCN(c4ncc5cnn(C6COC6)c5n4)CC3)C2)cc(C(F)(F)F)n1]

Followed the General procedure A to afford the desired product as a white solid (10 mg, 39%). ¹H NMR (500 MHz, CDCl₃) δ 8.75 (s, 1H), 7.93 (d, J=0.5 Hz, 1H), 6.34 (s, 1H), 5.94 (tt, J=7.9, 6.6 Hz, 1H), 5.29 (t, J=6.6 Hz, 2H), 5.10-5.00 (m, 2H), 3.94 (d, J=63.2 Hz, 8H), 2.57 (s, 3H), 1.91 (t, J=5.8 Hz, 4H). MS m/z: 461 [M+H]⁺.

### Example 563: 2-[2-methyl-6-(trifluoromethyl)pyrimidin-4-yl]-7-[1-(oxetan-3-yl)-1H-pyrazolo[3,4-b]pyrazin-6-yl]-2,7-diazaspiro[3.5]nonane

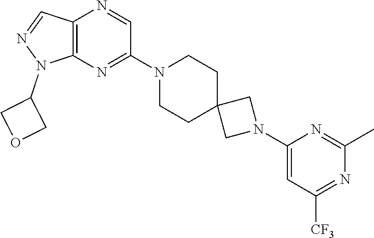 [SMILES: Cc1nc(N2CC3(CCN(c4cnc5cnn(C6COC6)c5n4)CC3)C2)cc(C(F)(F)F)n1]

Followed the General procedure A to afford the desired product as a white solid (14 mg, 57%). ¹H NMR (500 MHz, CDCl₃) δ 8.25 (s, 1H), 8.10 (s, 1H), 6.35 (s, 1H), 6.00-5.89 (m, 1H), 5.33 (t, J=6.6 Hz, 2H), 5.06 (dd, J=7.9, 6.7 Hz, 2H), 3.86 (dd, J=88.4, 49.9 Hz, 8H), 2.58 (s, 3H), 1.98 (t, J=5.7 Hz, 4H). MS m/z: 461 [M+H]⁺.

### Example 564: 7-[1-(2,2-difluoroethyl)-1H-pyrazolo[3,4-d]pyrimidin-6-yl]-2-[2-methyl-6-(trifluoromethyl)pyrimidin-4-yl]-2,7-diazaspiro[3.5]nonane

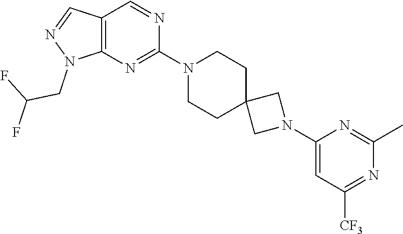 [SMILES: Cc1nc(N2CC3(CCN(c4ncc5cnn(CC(F)F)c5n4)CC3)C2)cc(C(F)(F)F)n1]

Followed the General procedure A to afford the desired product as a white solid (11.6 mg, 45%). ¹H NMR (500 MHz, CDCl₃) δ 8.76 (s, 1H), 7.88 (s, 1H), 6.34 (s, 1H), 6.21 (tt, J=55.6, 4.4 Hz, 1H), 4.63 (td, J=13.4, 4.5 Hz, 2H), 3.94 (d, J=64.7 Hz, 8H), 2.57 (s, 3H), 1.92 (d, J=5.8 Hz, 4H). MS m/z: 469 [M+H]⁺.

### Example 565: 7-[3-methyl-1-(oxetan-3-yl)-1H-pyrazolo[3,4-b]pyrazin-6-yl]-2-[2-methyl-6-(trifluoromethyl)pyrimidin-4-yl]-2,7-diazaspiro[3.5]nonane

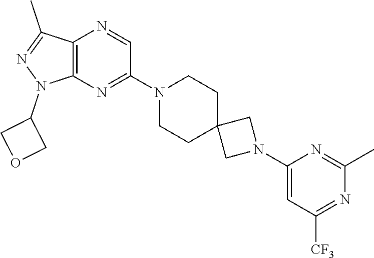 [SMILES: Cc1nc(N2CC3(CCN(c4cnc5c(C)nn(C6COC6)c5n4)CC3)C2)cc(C(F)(F)F)n1]

Followed the General procedure A to afford the desired product as a white solid (12.2 mg, 47%). ¹H NMR (500 MHz, CDCl₃) δ 8.17 (s, 1H), 6.34 (s, 1H), 5.87 (tt, J=7.9, 6.7 Hz, 1H), 5.32 (t, J=6.6 Hz, 2H), 5.04 (dd, J=7.9, 6.6 Hz, 2H), 3.85 (dd, J=96.5, 49.3 Hz, 8H), 2.59 (s, 3H), 2.57 (s, 3H), 1.97 (t, J=5.7 Hz, 4H). MS m/z: 475 [M+H]⁺.

### Example 566: 2-[2-methyl-6-(trifluoromethyl)pyrimidin-4-yl]-7-[1-(2,2,2-trifluoroethyl)-1H-pyrazolo[3,4-d]pyrimidin-6-yl]-2,7-diazaspiro[3.5]nonane

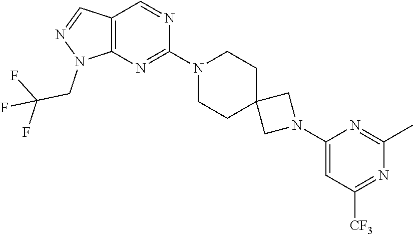 [SMILES: Cc1nc(N2CC3(CCN(c4ncc5cnn(CC(F)(F)F)c5n4)CC3)C2)cc(C(F)(F)F)n1]

Followed the General procedure A to afford the desired product as a white solid (18 mg, 68%). ¹H NMR (500 MHz, CDCl₃) δ 8.76 (s, 1H), 7.92 (s, 1H), 6.34 (s, 1H), 4.86 (q, J=8.4 Hz, 2H), 3.94 (d, J=65.3 Hz, 8H), 2.57 (s, 3H), 1.91 (t, J=5.7 Hz, 4H). MS m/z: 487 [M+H]⁺.

### Example 567: 2-[2-methyl-6-(trifluoromethyl)pyrimidin-4-yl]-7-[1-(2,2,2-trifluoroethyl)-1H-pyrazolo[3,4-b]pyrazin-6-yl]-2,7-diazaspiro[3.5]nonane

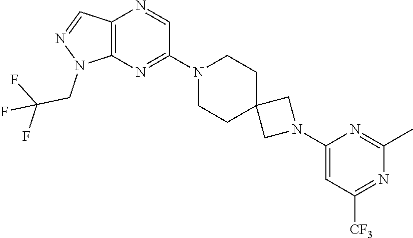 [SMILES: Cc1nc(N2CC3(CCN(c4cnc5cnn(CC(F)(F)F)c5n4)CC3)C2)cc(C(F)(F)F)n1]

Followed the General procedure A to afford the desired product as a white solid (13.3 mg, 50%). ¹H NMR (500 MHz, CDCl₃) δ 8.27 (s, 1H), 8.08 (s, 1H), 6.35 (s, 1H), 4.89 (q, J=8.4 Hz, 2H), 3.87 (dd, J=81.1, 50.3 Hz, 8H), 2.57 (s, 3H), 1.98 (t, J=5.7 Hz, 4H). MS m/z: 487 [M+H]⁺.

### Example 568: 7-[1-(2,2-difluoroethyl)-3-methyl-1H-pyrazolo[3,4-b]pyrazin-6-yl]-2-[2-methyl-6-(trifluoromethyl)pyrimidin-4-yl]-2,7-diazaspiro[3.5]nonane

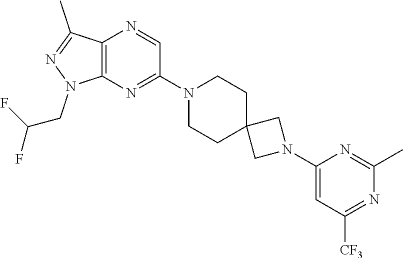 [SMILES: Cc1nc(N2CC3(CCN(c4cnc5c(C)nn(CC(F)F)c5n4)CC3)C2)cc(C(F)(F)F)n1]

Followed the General procedure A to afford the desired product as a white solid (14 mg, 53%). ¹H NMR (500 MHz, CDCl₃) δ 8.18 (s, 1H), 6.34 (s, 1H), 6.19 (tt, J=55.7, 4.5 Hz, 1H), 4.59 (td, J=13.4, 4.5 Hz, 2H), 3.85 (dd, J=91.4, 50.4 Hz, 8H), 2.57 (s, 3H), 2.55 (s, 3H), 1.97 (t, J=5.6 Hz, 4H). MS m/z: 483 [M+H]⁺.

### Example 569: 7-[1-(2,2-difluoroethyl)-3-methyl-1H-pyrazolo[3,4-d]pyrimidin-6-yl]-2-[2-methyl-6-(trifluoromethyl)pyrimidin-4-yl]-2,7-diazaspiro[3.5]nonane

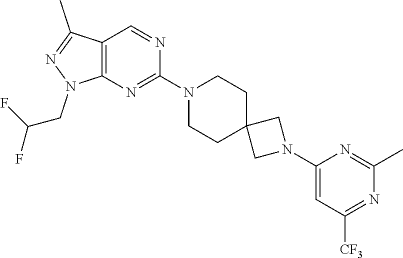 [SMILES: Cc1nc(N2CC3(CCN(c4ncc5c(C)nn(CC(F)F)c5n4)CC3)C2)cc(C(F)(F)F)n1]

Followed the General procedure A to afford the desired product as a white solid (17.6 mg, 67%). ¹H NMR (500 MHz, CDCl₃) δ 8.66 (s, 1H), 6.34 (s, 1H), 6.31-6.03 (m, 1H), 4.54 (td, J=13.4, 4.5 Hz, 2H), 3.93 (d, J=64.5 Hz, 8H), 2.57 (s, 3H), 2.48 (s, 3H), 1.90 (t, J=5.8 Hz, 4H). MS m/z: 483 [M+H]⁺.

### Example 570: 7-[3-methyl-1-(oxetan-3-yl)-1H-pyrazolo[3,4-d]pyrimidin-6-yl]-2-[2-methyl-6-(trifluoromethyl)pyrimidin-4-yl]-2,7-diazaspiro[3.5]nonane

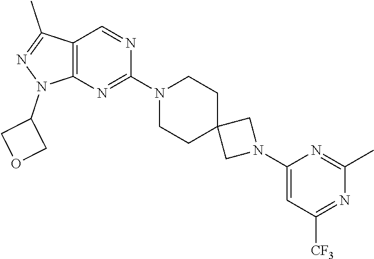 [SMILES: Cc1nc(N2CC3(CCN(c4ncc5c(C)nn(C6COC6)c5n4)CC3)C2)cc(C(F)(F)F)n1]

Followed the General procedure A to afford the desired product as a white solid (14.5 mg, 56%). ¹H NMR (500 MHz, CDCl₃) δ 8.65 (s, 1H), 6.34 (s, 1H), 5.86 (tt, J=7.9, 6.7 Hz, 1H), 5.27 (t, J=6.7 Hz, 2H), 5.08-4.96 (m, 2H), 3.93 (d, J=64.1 Hz, 8H), 2.57 (s, 3H), 2.52 (s, 3H), 1.90 (t, J=5.6 Hz, 4H). MS m/z: 475 [M+H]⁺.

### Example 571: 2-[2-methyl-6-(trifluoromethyl)pyrimidin-4-yl]-8-[1-(2,2,2-trifluoroethyl)-1H-pyrazolo[3,4-b]pyrazin-6-yl]-2,8-diazaspiro[4.5]decane

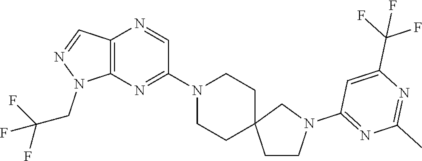 [SMILES: Cc1nc(N2CCC3(CCN(c4cnc5cnn(CC(F)(F)F)c5n4)CC3)C2)cc(C(F)(F)F)n1]

Followed the General procedure A to afford the desired product as a white solid (16.3 mg, 60%). ¹H NMR (500 MHz, CDCl₃) δ 8.65 (s, 1H), 6.34 (s, 1H), 5.86 (tt, J=7.9, 6.7 Hz, 1H), 5.27 (t, J=6.7 Hz, 2H), 5.08-4.96 (m, 2H), 3.93 (d, J=64.1 Hz, 8H), 2.57 (s, 3H), 2.52 (s, 3H), 1.90 (t, J=5.6 Hz, 4H). MS m/z: 501 [M+H]⁺.

### Example 572: 2-[2-methyl-6-(trifluoromethyl)pyrimidin-4-yl]-8-[1-(2,2,2-trifluoroethyl)-1H-pyrazolo[3,4-d]pyrimidin-6-yl]-2,8-diazaspiro[4.5]decane

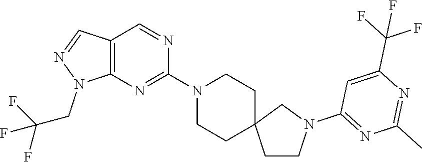 [SMILES: Cc1nc(N2CCC3(CCN(c4ncc5cnn(CC(F)(F)F)c5n4)CC3)C2)cc(C(F)(F)F)n1]

Followed the General procedure A to afford the desired product as a white solid (17.6 mg, 49%). ¹H NMR (500 MHz, CDCl₃) δ 8.76 (s, 1H), 7.91 (s, 1H), 6.44 (d, J=6.6 Hz, 1H), 4.85 (q, J=8.4 Hz, 2H), 4.13 (dt, J=11.5, 5.0 Hz, 1H), 4.07-3.90 (m, 2H), 3.90-3.73 (m, 2H), 3.64 (s, 1H), 3.51 (t, J=7.1 Hz, 1H), 3.31 (s, 1H), 2.58 (s, 3H), 2.00 (dt, J=27.4, 7.1 Hz, 2H), 1.71 (dd, J=12.0, 6.4 Hz, 4H). MS m/z: 501 [M+H]⁺.

### Example 573: 2-[2-methyl-6-(trifluoromethyl)pyrimidin-4-yl]-8-[1-(oxetan-3-yl)-1H-pyrazolo[3,4-d]pyrimidin-6-yl]-2,8-diazaspiro[4.5]decane

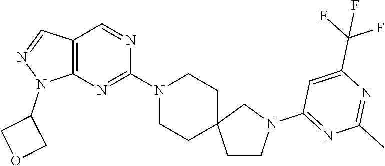 [SMILES: Cc1nc(N2CCC3(CCN(c4ncc5cnn(C6COC6)c5n4)CC3)C2)cc(C(F)(F)F)n1]

Followed the General procedure A to afford the desired product as a white solid (8.8 mg, 26%). ¹H NMR (500 MHz, CDCl₃) δ 8.75 (s, 1H), 7.93 (s, 1H), 6.44 (d, J=8.3 Hz, 1H), 5.94 (p, J=7.2 Hz, 1H), 5.29 (t, J=6.6 Hz, 2H), 5.05 (dd, J=7.9, 6.6 Hz, 2H), 4.18-4.05 (m, 1H), 4.03-3.89 (m, 2H), 3.88-3.72 (m, 2H), 3.64 (s, 1H), 3.51 (t, J=7.1 Hz, 1H), 3.30 (s, 1H), 2.58 (s, 3H), 2.00 (dt, J=28.5, 7.0 Hz, 2H), 1.76-1.63 (m, 4H). MS m/z: 475 [M+H]⁺.

### Example 574: 2-[2-methyl-6-(trifluoromethyl)pyrimidin-4-yl]-8-[1-(oxetan-3-yl)-1H-pyrazolo[3,4-b]pyrazin-6-yl]-2,8-diazaspiro[4.5]decane

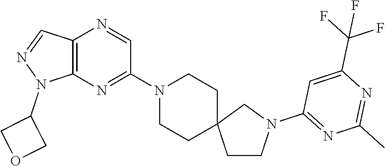 [SMILES: Cc1nc(N2CCC3(CCN(c4cnc5cnn(C6COC6)c5n4)CC3)C2)cc(C(F)(F)F)n1]

Followed the General procedure A to afford the desired product as a white solid (16.2 mg, 47%). ¹H NMR (500 MHz, CDCl₃) δ 8.24 (s, 1H), 8.09 (s, 1H), 6.44 (d, J=10.9 Hz, 1H), 6.00-5.85 (m, 1H), 5.34 (t, J=6.6 Hz, 2H), 5.12-5.00 (m, 2H), 3.97-3.64 (m, 6H), 3.52 (t, J=7.0 Hz, 1H), 3.32 (s, 1H), 2.58 (s, 3H), 2.02 (dt, J=19.4, 7.2 Hz, 2H), 1.81-1.70 (m, 4H). MS m/z: 475 [M+H]⁺.

### Example 575: 8-[1-(2,2-difluoroethyl)-3-methyl-1H-pyrazolo[3,4-b]pyrazin-6-yl]-2-[2-methyl-6-(trifluoromethyl)pyrimidin-4-yl]-2,8-diazaspiro[4.5]decane

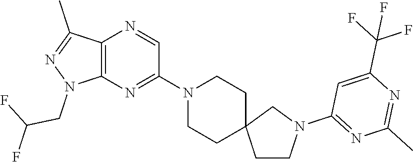 [SMILES: Cc1nc(N2CCC3(CCN(c4cnc5c(C)nn(CC(F)F)c5n4)CC3)C2)cc(C(F)(F)F)n1]

Followed the General procedure A to afford the desired product as a white solid (18 mg, 50%). ¹H NMR (500 MHz, CDCl₃) δ 8.18 (s, 1H), 6.44 (d, J=8.7 Hz, 1H), 6.19 (tt, J=55.7, 4.5 Hz, 1H), 4.59 (td, J=13.4, 4.5 Hz, 2H), 3.97-3.61 (m, 6H), 3.52 (t, J=7.0 Hz, 1H), 3.32 (s, 1H), 2.58 (s, 3H), 2.55 (s, 3H), 2.09-1.91 (m, 2H), 1.78 (dd, J=13.3, 7.2 Hz, 4H). MS m/z: 497 [M+H]⁺.

### Example 576: 8-[1-(2,2-difluoroethyl)-3-methyl-1H-pyrazolo[3,4-d]pyrimidin-6-yl]-2-[2-methyl-6-(trifluoromethyl)pyrimidin-4-yl]-2,8-diazaspiro[4.5]decane

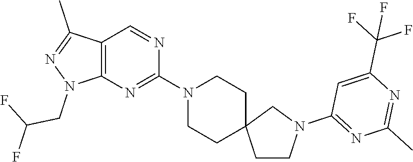 [SMILES: Cc1nc(N2CCC3(CCN(c4ncc5c(C)nn(CC(F)F)c5n4)CC3)C2)cc(C(F)(F)F)n1]

Followed the General procedure A to afford the desired product as a white solid (13 mg, 36%). ¹H NMR (500 MHz, CDCl₃) δ 8.66 (s, 1H), 6.44 (d, J=6.5 Hz, 1H), 6.19 (tt, J=55.7, 4.5 Hz, 1H), 4.54 (td, J=13.4, 4.5 Hz, 2H), 4.16-4.07 (m, 1H), 4.04-3.90 (m, 2H), 3.88-3.74 (m, 2H), 3.63 (s, 1H), 3.51 (t, J=7.1 Hz, 1H), 3.30 (s, 1H), 2.58 (s, 3H), 2.48 (s, 3H), 2.06-1.93 (m, 2H), 1.69 (dq, J=13.3, 8.4, 7.1 Hz, 4H). MS m/z: 497 [M+H]⁺.

### Example 577: 2-[1-(2,2-difluoroethyl)-3-methyl-1H-pyrazolo[3,4-d]pyrimidin-6-yl]-6-[2-methyl-6-(trifluoromethyl)pyrimidin-4-yl]-2,6-diazaspiro[3.4]octane

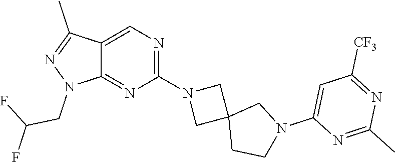 [SMILES: Cc1nc(N2CCC3(C2)CN(c2ncc4c(C)nn(CC(F)F)c4n2)C3)cc(C(F)(F)F)n1]

Followed the General procedure A to afford the desired product as a white solid (15.1 mg, 42%). ¹H NMR (500 MHz, CDCl₃) δ 8.68 (s, 1H), 6.45 (d, J=11.1 Hz, 1H), 6.18 (tt, J=55.7, 4.5 Hz, 1H), 4.55 (td, J=13.3, 4.5 Hz, 2H), 4.24-4.11 (m, 4H), 3.93 (s, 1H), 3.79 (s, 1H), 3.66 (s, 1H), 3.53 (s, 1H), 2.57 (s, 3H), 2.49 (s, 3H), 2.45-2.25 (m, 2H). MS m/z: 469 [M+H]⁺.

### Example 578: 2-[1-(2,2-difluoroethyl)-3-methyl-1H-pyrazolo[3,4-b]pyrazin-6-yl]-6-[2-methyl-6-(trifluoromethyl)pyrimidin-4-yl]-2,6-diazaspiro[3.4]octane

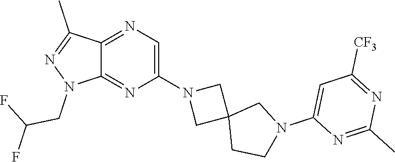 [SMILES: Cc1nc(N2CCC3(C2)CN(c2cnc4c(C)nn(CC(F)F)c4n2)C3)cc(C(F)(F)F)n1]

Followed the General procedure A to afford the desired product as a white solid (15 mg, 42%). ¹H NMR (500 MHz, CDCl₃) δ 7.74 (s, 1H), 6.46 (s, 1H), 6.19 (tt, J=55.7, 4.5 Hz, 1H), 4.58 (td, J=13.3, 4.5 Hz, 2H), 4.26-4.17 (m, 4H), 3.96 (s, 1H), 3.81 (s, 1H), 3.69 (s, 1H), 3.54 (s, 1H), 2.58 (d, J=3.2 Hz, 3H), 2.55 (s, 3H), 2.38 (d, J=42.4 Hz, 2H). MS m/z: 469 [M+H]⁺.

### Example 579: 2-[1-(2,2-difluoroethyl)-1H-pyrazolo[3,4-b]pyrazin-6-yl]-8-[2-methyl-6-(trifluoromethyl)pyrimidin-4-yl]-2,8-diazaspiro[4.5]decane

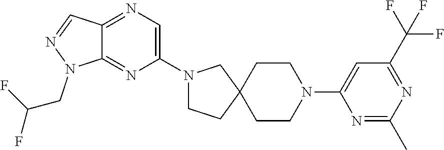 [SMILES: Cc1nc(N2CCC3(CC2)CCN(c2cnc4cnn(CC(F)F)c4n2)C3)cc(C(F)(F)F)n1]

Followed the General procedure A to afford the desired product as a white solid (19 mg, 51%). ¹H NMR (500 MHz, CDCl₃) δ 8.05 (s, 1H), 7.96 (s, 1H), 6.68 (s, 1H), 6.23 (tt, J=55.7, 4.5 Hz, 1H), 4.67 (td, J=13.3, 4.5 Hz, 2H), 3.74 (t, J=7.1 Hz, 6H), 3.57 (s, 2H), 2.57 (s, 3H), 2.05 (t, J=7.0 Hz, 2H), 1.79-1.69 (m, 4H). MS m/z: 483 [M+H]⁺.

### Example 580: 8-[2-methyl-6-(trifluoromethyl)pyrimidin-4-yl]-2-[1-(2,2,2-trifluoroethyl)-1H-pyrazolo[3,4-b]pyrazin-6-yl]-2,8-diazaspiro[4.5]decane

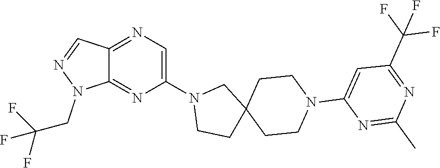 [SMILES: Cc1nc(N2CCC3(CC2)CCN(c2cnc4cnn(CC(F)(F)F)c4n2)C3)cc(C(F)(F)F)n1]

Followed the General procedure A to afford the desired product as a white solid (16.2 mg, 42%). ¹H NMR (500 MHz, CDCl₃) δ 8.09 (s, 1H), 7.98 (s, 1H), 6.68 (s, 1H), 4.90 (q, J=8.4 Hz, 2H), 3.91-3.65 (m, 6H), 3.57 (s, 2H), 2.57 (s, 3H), 2.06 (t, J=7.0 Hz, 2H), 1.81-1.70 (m, 4H). MS m/z: 501 [M+H]⁺.

### Example 581: 2-[1-(2,2-difluoroethyl)-1H-pyrazolo[3,4-d]pyrimidin-6-yl]-8-[2-methyl-6-(trifluoromethyl)pyrimidin-4-yl]-2,8-diazaspiro[4.5]decane

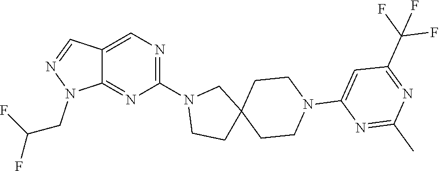 [SMILES: Cc1nc(N2CCC3(CC2)CCN(c2ncc4cnn(CC(F)F)c4n2)C3)cc(C(F)(F)F)n1]

Followed the General procedure A to afford the desired product as a white solid (6.4 mg, 17%). ¹H NMR (500 MHz, CDCl₃) δ 8.78 (s, 1H), 7.89 (s, 1H), 6.67 (s, 1H), 6.23 (tt, J=55.5, 4.6 Hz, 1H), 4.64 (td, J=13.4, 4.5 Hz, 2H), 4.02-3.54 (m, 8H), 2.56 (s, 3H), 1.97 (t, J=7.1 Hz, 2H), 1.72 (dtt, J=21.8, 7.7, 4.0 Hz, 4H). MS m/z: 483 [M+H]⁺.

### Example 582: 2-[1-(2,2-difluoroethyl)-3-methyl-1H-pyrazolo[3,4-b]pyrazin-6-yl]-8-[2-methyl-6-(trifluoromethyl)pyrimidin-4-yl]-2,8-diazaspiro[4.5]decane

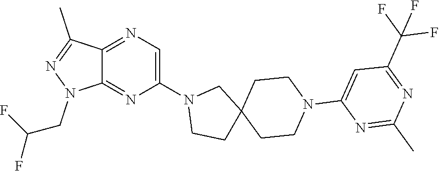 [SMILES: Cc1nc(N2CCC3(CC2)CCN(c2cnc4c(C)nn(CC(F)F)c4n2)C3)cc(C(F)(F)F)n1]

Followed the General procedure A to afford the desired product as a white solid (8.2 mg, 21%). ¹H NMR (500 MHz, CDCl₃) δ 7.88 (s, 1H), 6.68 (s, 1H), 6.21 (tt, J=55.8, 4.5 Hz, 2H), 4.60 (td, J=13.4, 4.5 Hz, 2H), 3.92-3.67 (m, 6H), 3.56 (s, 2H), 2.57 (s, 3H), 2.55 (s, 3H), 2.04 (t, J=7.0 Hz, 2H), 1.75 (ddt, J=17.0, 13.6, 6.7 Hz, 4H). MS m/z: 497 [M+H]⁺.

### Example 583: 8-[2-methyl-6-(trifluoromethyl)pyrimidin-4-yl]-2-[1-(oxetan-3-yl)-1H-pyrazolo[3,4-b]pyrazin-6-yl]-2,8-diazaspiro[4.5]decane

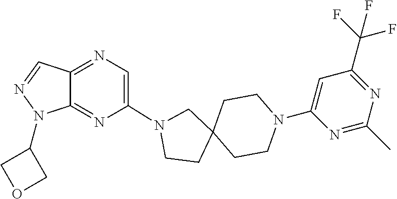 [SMILES: Cc1nc(N2CCC3(CC2)CCN(c2cnc4cnn(C5COC5)c4n2)C3)cc(C(F)(F)F)n1]

Followed the General procedure A to afford the desired product as a white solid (16.5 mg, 45%). ¹H NMR (500 MHz, CDCl₃) δ 8.10 (s, 1H), 7.95 (s, 1H), 6.68 (s, 1H), 5.96 (p, J=7.3 Hz, 1H), 5.34 (t, J=6.6 Hz, 2H), 5.06 (dd, J=7.9, 6.5 Hz, 2H), 3.88-3.67 (m, 6H), 3.56 (s, 2H), 2.57 (s, 3H), 2.05 (t, J=7.0 Hz, 2H), 1.74 (td, J=7.7, 4.4 Hz, 4H). MS m/z: 475 [M+H]⁺.

### Example 584: 7-[2-methyl-6-(trifluoromethyl)pyrimidin-4-yl]-2-[1-(oxetan-3-yl)-1H-pyrazolo[3,4-b]pyrazin-6-yl]-2,7-diazaspiro[3.5]nonane

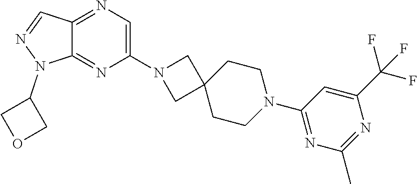 [SMILES: Cc1nc(N2CCC3(CC2)CN(c2cnc4cnn(C5COC5)c4n2)C3)cc(C(F)(F)F)n1]

Followed the General procedure A to afford the desired product as a white solid (16.4 mg, 47%). ¹H NMR (500 MHz, CDCl₃) δ 8.11 (s, 1H), 7.80 (s, 1H), 6.69 (s, 1H), 5.94 (tt, J=7.8, 6.6 Hz, 1H), 5.32 (t, J=6.6 Hz, 2H), 5.05 (dd, J=7.9, 6.6 Hz, 2H), 4.02 (s, 4H), 3.76 (s, 4H), 2.57 (s, 3H), 1.99-1.90 (m, 4H). MS m/z: 461 [M+H]⁺.

### Example 585: 2-[1-(2,2-difluoroethyl)-3-methyl-1H-pyrazolo[3,4-b]pyrazin-6-yl]-7-[2-methyl-6-(trifluoromethyl)pyrimidin-4-yl]-2,7-diazaspiro[3.5]nonane

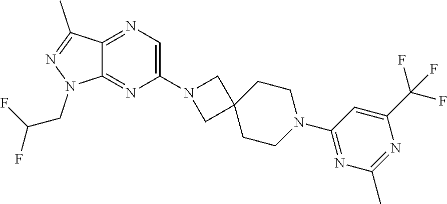 [SMILES: Cc1nc(N2CCC3(CC2)CN(c2cnc4c(C)nn(CC(F)F)c4n2)C3)cc(C(F)(F)F)n1]

Followed the General procedure A to afford the desired product as a white solid (16.6 mg, 44%). ¹H NMR (500 MHz, CDCl₃) δ 7.74 (s, 1H), 6.69 (s, 1H), 6.34-6.05 (m, 1H), 4.58 (td, J=13.3, 4.5 Hz, 2H), 4.01 (s, 4H), 3.75 (s, 4H), 2.57 (s, 3H), 2.55 (s, 3H), 1.98-1.92 (m, 4H). MS m/z: 483 [M+H]⁺.

### Example 586: 2-[1-(2,2-difluoroethyl)-1H-pyrazolo[3,4-d]pyrimidin-6-yl]-7-[2-methyl-6-(trifluoromethyl)pyrimidin-4-yl]-2,7-diazaspiro[3.5]nonane

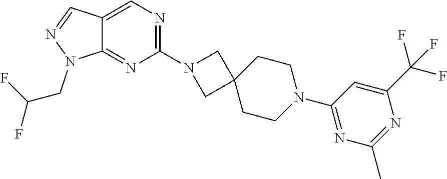 [SMILES: Cc1nc(N2CCC3(CC2)CN(c2ncc4cnn(CC(F)F)c4n2)C3)cc(C(F)(F)F)n1]

Followed the General procedure A to afford the desired product as a white solid (11.5 mg, 32%). ¹H NMR (500 MHz, CDCl₃) δ 8.77 (s, 1H), 7.91 (s, 1H), 6.69 (s, 1H), 6.22 (tt, J=55.6, 4.5 Hz, 1H), 4.63 (td, J=13.3, 4.5 Hz, 2H), 4.00 (s, 4H), 3.74 (s, 4H), 2.56 (s, 3H), 1.97-1.88 (m, 4H). MS m/z: 469 [M+H]⁺.

### Example 587: 7-[2-methyl-6-(trifluoromethyl)pyrimidin-4-yl]-2-[1-(2,2,2-trifluoroethyl)-1H-pyrazolo[3,4-b]pyrazin-6-yl]-2,7-diazaspiro[3.5]nonane

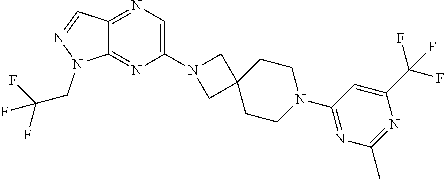 [SMILES: Cc1nc(N2CCC3(CC2)CN(c2cnc4cnn(CC(F)(F)F)c4n2)C3)cc(C(F)(F)F)n1]

Followed the General procedure A to afford the desired product as a white solid (16.7 mg, 44%). ¹H NMR (500 MHz, CDCl₃) δ 8.09 (s, 1H), 7.82 (s, 1H), 6.69 (s, 1H), 4.89 (q, J=8.4 Hz, 2H), 4.02 (s, 4H), 3.76 (s, 2H), 2.57 (s, 3H), 1.99-1.93 (m, 4H). MS m/z: 487 [M+H]⁺.

### Example 588: 2-[1-(2,2-difluoroethyl)-1H-pyrazolo[3,4-b]pyrazin-6-yl]-7-[2-methyl-6-(trifluoromethyl)pyrimidin-4-yl]-2,7-diazaspiro[3.5]nonane

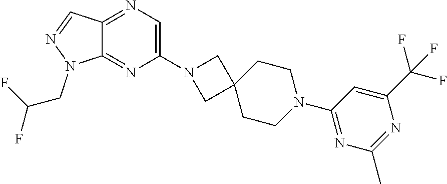 [SMILES: Cc1nc(N2CCC3(CC2)CN(c2cnc4cnn(CC(F)F)c4n2)C3)cc(C(F)(F)F)n1]

Followed the General procedure A to afford the desired product as a white solid (18.4 mg, 51%). ¹H NMR (500 MHz, CDCl₃) δ 8.06 (s, 1H), 7.81 (s, 1H), 6.69 (s, 1H), 6.22 (t, J=4.5 Hz, 1H), 4.66 (td, J=13.3, 4.5 Hz, 2H), 4.02 (s, 4H), 3.76 (s, 2H), 2.57 (s, 3H), 1.98-1.93 (m, 4H). MS m/z: 469 [M+H]⁺.

### Example 589: 2-[2-methyl-6-(trifluoromethyl)pyrimidin-4-yl]-6-[1-(oxetan-3-yl)-1H-pyrazolo[3,4-d]pyrimidin-6-yl]-2,6-diazaspiro[3.4]octane

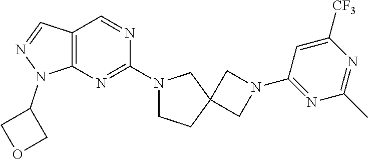 [SMILES: Cc1nc(N2CC3(CCN(c4ncc5cnn(C6COC6)c5n4)C3)C2)cc(C(F)(F)F)n1]

Followed the General procedure A to afford the desired product as a white solid (8.9 mg, 37%). ¹H NMR (500 MHz, CDCl₃) δ 8.79 (s, 1H), 7.98 (s, 1H), 6.34 (s, 1H), 5.94 (p, J=7.3 Hz, 1H), 5.31 (t, J=6.6 Hz, 2H), 5.06 (dd, J=7.9, 6.6 Hz, 2H), 4.16 (s, 4H), 3.93 (s, 2H), 3.80 (s, 2H), 2.58 (s, 3H), 2.33 (t, J=6.9 Hz, 2H). MS m/z: 447 [M+H]⁺.

### Example 590: 2-[6-methyl-2-(trifluoromethyl)pyrimidin-4-yl]-6-[1-(oxetan-3-yl)-1H-pyrazolo[3,4-d]pyrimidin-6-yl]-2,6-diazaspiro[3.4]octane

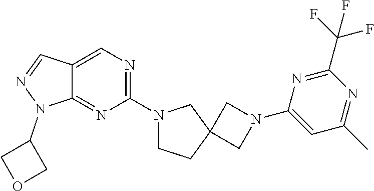 [SMILES: Cc1cc(N2CC3(CCN(c4ncc5cnn(C6COC6)c5n4)C3)C2)nc(C(F)(F)F)n1]

Followed the General procedure A to afford the desired product as a white solid (7.1 mg, 14%). ¹H NMR (500 MHz, CDCl₃) δ 8.77 (s, 1H), 7.95 (s, 1H), 6.13 (s, 1H), 5.94 (p, J=7.3 Hz, 1H), 5.31 (t, J=6.6 Hz, 2H), 5.06 (t, J=7.2 Hz, 2H), 4.13 (s, 4H), 3.91 (s, 2H), 3.77 (s, 2H), 2.43 (s, 3H), 2.31 (t, J=6.9 Hz, 2H). MS m/z: 447 [M+H]⁺.

### Example 591: 6-[1-(2,2-difluoroethyl)-3-methyl-1H-pyrazolo[3,4-d]pyrimidin-6-yl]-2-[2-methyl-4-(trifluoromethyl)pyrimidin-5-yl]-2,6-diazaspiro[3.4]octane

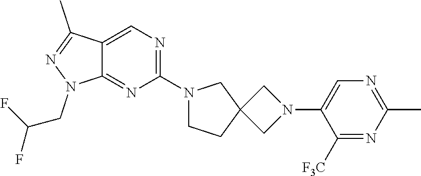 [SMILES: Cc1ncc(N2CC3(CCN(c4ncc5c(C)nn(CC(F)F)c5n4)C3)C2)c(C(F)(F)F)n1]

Followed the General procedure A to afford the desired product as a white solid (14 mg, 38.48%). ¹H NMR (500 MHz, CDCl₃) δ 8.68 (s, 1H), 8.09 (s, 1H), 6.19 (tt, J=55.7, 4.5 Hz, 1H), 4.55 (td, J=13.4, 4.5 Hz, 2H), 4.19-4.06 (m, 4H), 3.91 (s, 2H), 3.77 (s, 2H), 2.67 (s, 3H), 2.49 (s, 3H), 2.32 (t, J=6.9 Hz, 2H). MS m/z: 469 [M+H]⁺.

### Example 592: 6-[1-(2,2-difluoroethyl)-1H-pyrazolo[3,4-b]pyrazin-6-yl]-2-[2-methyl-4-(trifluoromethyl)pyrimidin-5-yl]-2,6-diazaspiro[3.4]octane

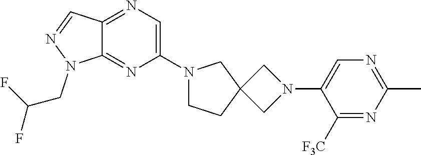 [SMILES: Cc1ncc(N2CC3(CCN(c4cnc5cnn(CC(F)F)c5n4)C3)C2)c(C(F)(F)F)n1]

Followed the General procedure A to afford the desired product as a white solid (16 mg, 45.34%). ¹H NMR (500 MHz, CDCl₃) δ 8.10 (s, 1H), 8.06 (s, 1H), 7.97 (s, 1H), 6.22 (tt, J=55.7, 4.5 Hz, 1H), 4.67 (td, J=13.3, 4.5 Hz, 2H), 4.20-4.10 (m, 4H), 3.90 (s, 2H), 3.75 (t, J=6.9 Hz, 2H), 2.68 (s, 3H), 2.40 (t, J=6.9 Hz, 2H). MS m/z: 455 [M+H]⁺.

### Example 593: 6-[1-(2,2-difluoroethyl)-3-methyl-1H-pyrazolo[3,4-b]pyrazin-6-yl]-2-[2-methyl-4-(trifluoromethyl)pyrimidin-5-yl]-2,6-diazaspiro[3.4]octane

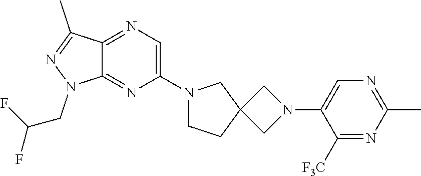 [SMILES: Cc1ncc(N2CC3(CCN(c4cnc5c(C)nn(CC(F)F)c5n4)C3)C2)c(C(F)(F)F)n1]

Followed the General procedure A to afford the desired product as a white solid (11 mg, 30.24%). ¹H NMR (500 MHz, CDCl₃) δ 8.09 (s, 1H), 7.89 (s, 1H), 6.20 (tt, J=55.7, 4.5 Hz, 1H), 4.59 (td, J=13.4, 4.5 Hz, 2H), 4.21-4.09 (m, 4H), 3.88 (s, 2H), 3.74 (t, J=6.9 Hz, 2H), 2.67 (s, 3H), 2.55 (s, 3H), 2.39 (t, J=6.9 Hz, 2H). MS m/z: 469 [M+H]⁺.

### Example 594: 2-[2-methyl-4-(trifluoromethyl)pyrimidin-5-yl]-6-[1-(2,2,2-trifluoroethyl)-1H-pyrazolo[3,4-b]pyrazin-6-yl]-2,6-diazaspiro[3.4]octane

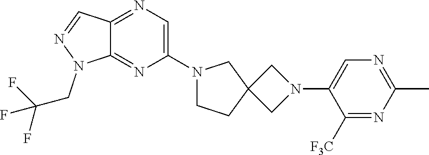 [SMILES: Cc1ncc(N2CC3(CCN(c4cnc5cnn(CC(F)(F)F)c5n4)C3)C2)c(C(F)(F)F)n1]

Followed the General procedure A to afford the desired product as a white solid (13 mg, 35.44%). ¹H NMR (500 MHz, CDCl₃) δ 8.10 (d, J=1.0 Hz, 2H), 7.98 (s, 1H), 4.89 (q, J=8.4 Hz, 2H), 4.22-4.09 (m, 4H), 3.89 (s, 2H), 3.75 (t, J=6.9 Hz, 2H), 2.68 (s, 3H), 2.40 (t, J=6.9 Hz, 2H). MS m/z: 473 [M+H]⁺.

### Example 595: 6-[1-(2,2-difluoroethyl)-1H-pyrazolo[3,4-d]pyrimidin-6-yl]-2-[2-methyl-4-(trifluoromethyl)pyrimidin-5-yl]-2,6-diazaspiro[3.4]octane

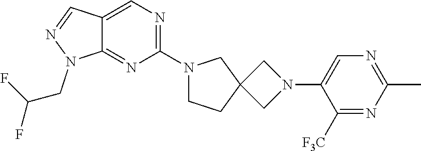 [SMILES: Cc1ncc(N2CC3(CCN(c4ncc5cnn(CC(F)F)c5n4)C3)C2)c(C(F)(F)F)n1]

Followed the General procedure A to afford the desired product as a white solid (8 mg, 22.67%). ¹H NMR (500 MHz, CDCl₃) δ 8.78 (s, 1H), 8.09 (s, 1H), 7.90 (s, 1H), 6.22 (tt, J=55.6, 4.5 Hz, 1H), 4.64 (td, J=13.4, 4.5 Hz, 2H), 4.17-4.09 (m, 4H), 3.92 (s, 2H), 3.78 (s, 2H), 2.67 (s, 3H), 2.33 (t, J=7.0 Hz, 2H). MS m/z: 455 [M+H]⁺.

### Example 596: 2-[2-methyl-4-(trifluoromethyl)pyrimidin-5-yl]-6-[1-(2,2,2-trifluoroethyl)-1H-pyrazolo[3,4-d]pyrimidin-6-yl]-2,6-diazaspiro[3.4]octane

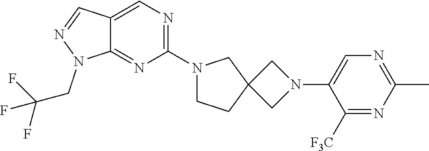 [SMILES: Cc1ncc(N2CC3(CCN(c4ncc5cnn(CC(F)(F)F)c5n4)C3)C2)c(C(F)(F)F)n1]

Followed the General procedure A to afford the desired product as a white solid (7 mg, 19.08%). ¹H NMR (500 MHz, CDCl₃) δ 8.79 (s, 1H), 8.09 (s, 1H), 7.94 (s, 1H), 4.87 (q, J=8.4 Hz, 2H), 4.12 (t, J=11.3 Hz, 4H), 3.92 (s, 2H), 3.78 (s, 2H), 2.67 (s, 3H), 2.33 (t, J=7.0 Hz, 2H). MS m/z: 473 [M+H]⁺.

### Example 597: 2-[2-methyl-4-(trifluoromethyl)pyrimidin-5-yl]-6-[1-(oxetan-3-yl)-1H-pyrazolo[3,4-b]pyrazin-6-yl]-2,6-diazaspiro[3.4]octane

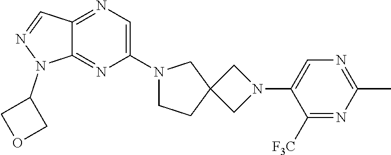 [SMILES: Cc1ncc(N2CC3(CCN(c4cnc5cnn(C6COC6)c5n4)C3)C2)c(C(F)(F)F)n1]

Followed the General procedure A to afford the desired product as a white solid (13 mg, 37.5%). ¹H NMR (500 MHz, CDCl₃) δ 8.10 (d, J=3.0 Hz, 2H), 7.96 (s, 1H), 5.94 (p, J=7.3 Hz, 1H), 5.35 (t, J=6.6 Hz, 2H), 5.06 (dd, J=7.9, 6.5 Hz, 2H), 4.19-4.10 (m, 4H), 3.89 (s, 2H), 3.74 (t, J=6.9 Hz, 2H), 2.68 (s, 3H), 2.40 (t, J=6.9 Hz, 2H). MS m/z: 447 [M+H]⁺.

### Example 598: 2-[2-methyl-4-(trifluoromethyl)pyrimidin-5-yl]-6-[1-(oxetan-3-yl)-1H-pyrazolo[3,4-d]pyrimidin-6-yl]-2,6-diazaspiro[3.4]octane

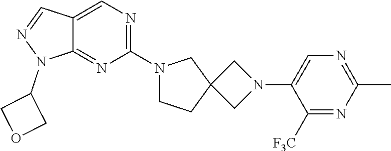 [SMILES: Cc1ncc(N2CC3(CCN(c4ncc5cnn(C6COC6)c5n4)C3)C2)c(C(F)(F)F)n1]

Followed the General procedure A to afford the desired product as a white solid (7 mg, 20.19%). ¹H NMR (500 MHz, CDCl₃) δ 8.77 (s, 1H), 8.09 (s, 1H), 7.95 (s, 1H), 5.94 (p, J=7.3 Hz, 1H), 5.31 (t, J=6.6 Hz, 2H), 5.05 (dd, J=7.9, 6.6 Hz, 2H), 4.18-4.05 (m, 4H), 3.91 (s, 2H), 3.77 (s, 2H), 2.67 (s, 3H), 2.33 (t, J=6.9 Hz, 2H). MS m/z: 447 [M+H]⁺.

### Example 599: 6-[3-methyl-1-(oxetan-3-yl)-1H-pyrazolo[3,4-b]pyrazin-6-yl]-2-[2-methyl-4-(trifluoromethyl)pyrimidin-5-yl]-2,6-diazaspiro[3.4]octane

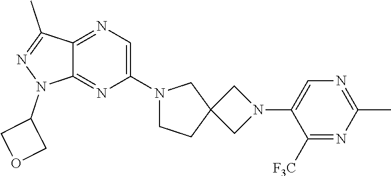 [SMILES: Cc1ncc(N2CC3(CCN(c4cnc5c(C)nn(C6COC6)c5n4)C3)C2)c(C(F)(F)F)n1]

Followed the General procedure A to afford the desired product as a white solid (12 mg, 33.56%). ¹H NMR (500 MHz, CDCl₃) δ 8.09 (s, 1H), 7.87 (s, 1H), 5.87 (tt, J=8.0, 6.8 Hz, 1H), 5.33 (t, J=6.6 Hz, 2H), 5.06-4.99 (m, 2H), 4.20-4.08 (m, 4H), 3.88 (s, 2H), 3.73 (t, J=6.9 Hz, 2H), 2.67 (s, 3H), 2.59 (s, 3H), 2.39 (t, J=6.9 Hz, 2H). MS m/z: 461 [M+H]⁺.

### Example 600: 6-[3-methyl-1-(oxetan-3-yl)-1H-pyrazolo[3,4-d]pyrimidin-6-yl]-2-[2-methyl-4-(trifluoromethyl)pyrimidin-5-yl]-2,6-diazaspiro[3.4]octane

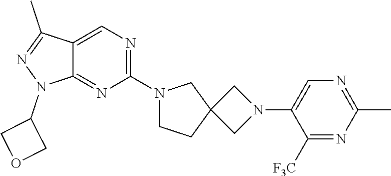 [SMILES: Cc1ncc(N2CC3(CCN(c4ncc5c(C)nn(C6COC6)c5n4)C3)C2)c(C(F)(F)F)n1]

Followed the General procedure A to afford the desired product as a white solid (14 mg, 39.15%). ¹H NMR (500 MHz, CDCl₃) δ 8.66 (s, 1H), 8.08 (s, 1H), 5.86 (tt, J=7.9, 6.7 Hz, 1H), 5.29 (t, J=6.6 Hz, 2H), 5.02 (dd, J=7.9, 6.5 Hz, 2H), 4.18-4.04 (m, 4H), 3.90 (s, 2H), 3.77 (d, J=8.4 Hz, 2H), 2.67 (s, 3H), 2.53 (s, 3H), 2.32 (t, J=6.9 Hz, 2H). MS m/z: 461 [M+H]⁺.

### Example 601: 8-[1-(2,2-difluoroethyl)-1H-pyrazolo[3,4-b]pyrazin-6-yl]-2-[2-methyl-4-(trifluoromethyl)pyrimidin-5-yl]-2,8-diazaspiro[4.5]decane

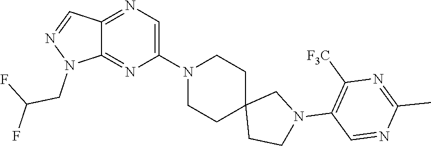 [SMILES: Cc1ncc(N2CCC3(CCN(c4cnc5cnn(CC(F)F)c5n4)CC3)C2)c(C(F)(F)F)n1]

Followed the General procedure A to afford the desired product as a white solid (23 mg, 65.84%). ¹H NMR (500 MHz, CDCl₃) δ 8.38 (s, 1H), 8.25 (s, 1H), 8.04 (s, 1H), 6.21 (tt, J=55.6, 4.5 Hz, 1H), 4.66 (td, J=13.4, 4.5 Hz, 2H), 3.80 (qdd, J=13.7, 6.9, 4.5 Hz, 4H), 3.56 (t, J=7.0 Hz, 2H), 3.33 (s, 2H), 2.67 (s, 3H), 1.98 (t, J=7.0 Hz, 2H), 1.84-1.70 (m, 4H). MS m/z: 483 [M+H]⁺.

### Example 602: 8-[1-(2,2-difluoroethyl)-3-methyl-1H-pyrazolo[3,4-b]pyrazin-6-yl]-2-[2-methyl-4-(trifluoromethyl)pyrimidin-5-yl]-2,8-diazaspiro[4.5]decane

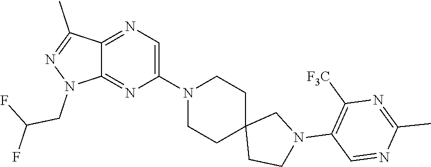 [SMILES: Cc1ncc(N2CCC3(CCN(c4cnc5c(C)nn(CC(F)F)c5n4)CC3)C2)c(C(F)(F)F)n1]

Followed the General procedure A to afford the desired product as a white solid (21 mg, 58.42%). ¹H NMR (500 MHz, CDCl₃) δ 8.38 (s, 1H), 8.17 (s, 1H), 6.19 (tt, J=55.7, 4.5 Hz, 1H), 4.58 (td, J=13.4, 4.5 Hz, 2H), 3.78 (qdd, J=13.7, 6.9, 4.5 Hz, 4H), 3.55 (t, J=7.0 Hz, 2H), 3.32 (s, 2H), 2.67 (s, 3H), 2.54 (s, 3H), 1.97 (t, J=7.0 Hz, 2H), 1.82-1.71 (m, 4H). MS m/z: 497 [M+H]⁺.

### Example 603: 2-[3-methyl-1-(2,2,2-trifluoroethyl)-1H-pyrazolo[3,4-b]pyrazin-6-yl]-7-[2-(trifluoromethyl)pyrimidin-5-yl]-2,7-diazaspiro[4.4]nonane

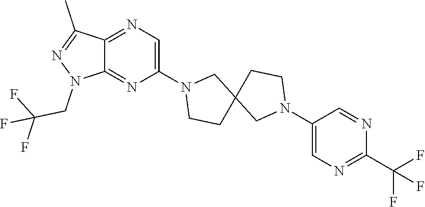 [SMILES: Cc1nn(CC(F)(F)F)c2nc(N3CCC4(CCN(c5cnc(C(F)(F)F)nc5)C4)C3)cnc12]

Followed the General procedure A to afford the desired product as a white solid (10.5 mg, 27.8%). ¹H NMR (500 MHz, CDCl₃) δ 8.12 (d, J=2.3 Hz, 2H), 7.91 (s, 1H), 4.81 (q, J=8.5 Hz, 2H), 3.80 (q, J=7.2 Hz, 2H), 3.70-3.56 (m, 4H), 3.46 (q, J=9.6 Hz, 2H), 2.57 (s, 3H), 2.29-2.16 (m, 4H). MS m/z: 487 [M+H]⁺.

### Example 604: 2-[3-methyl-1-(2,2,2-trifluoroethyl)-1H-pyrazolo[3,4-b]pyrazin-6-yl]-7-[2-methyl-6-(trifluoromethyl)pyrimidin-4-yl]-2,7-diazaspiro[4.4]nonane

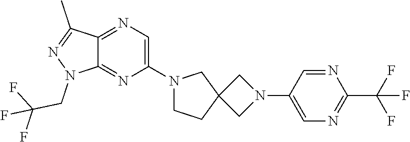 [SMILES: Cc1nn(CC(F)(F)F)c2nc(N3CCC4(CN(c5cnc(C(F)(F)F)nc5)C4)C3)cnc12]

Followed the General procedure A to afford the desired product as a white solid (9.8 mg, 26.13%). ¹H NMR (500 MHz, CDCl₃) δ 7.89 (s, 1H), 6.49 (s, 1H), 4.88-4.74 (m, 2H), 3.9-3.45 (m, 8H), 2.6-2.56 (m, 7H). MS m/z: 501 [M+H]⁺.

### Example 605: 6-[3-methyl-1-(2,2,2-trifluoroethyl)-1H-pyrazolo[3,4-b]pyrazin-6-yl]-2-[2-(trifluoromethyl)pyrimidin-5-yl]-2,6-diazaspiro[3.4]octane

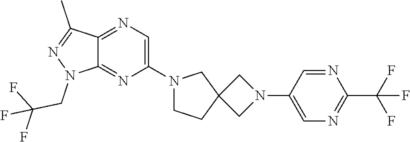 [SMILES: Cc1nn(CC(F)(F)F)c2nc(N3CCC4(CN(c5cnc(C(F)(F)F)nc5)C4)C3)cnc12]

Followed the General procedure A to afford the desired product as a white solid (13.2 mg, 37.29%). ¹H NMR (500 MHz, CDCl₃) δ 8.02 (d, J=1.3 Hz, 2H), 7.91 (s, 1H), 4.82 (q, J=8.5 Hz, 2H), 4.21-4.08 (m, 4H), 3.91 (s, 2H), 3.75 (t, J=6.9 Hz, 2H), 2.57 (s, 3H), 2.43 (t, J=6.9 Hz, 2H) MS m/z: 473 [M+H]⁺.

### Example 606: 6-[3-methyl-1-(2,2,2-trifluoroethyl)-1H-pyrazolo[3,4-b]pyrazin-6-yl]-2-[2-methyl-4-(trifluoromethyl)pyrimidin-5-yl]-2,6-diazaspiro[3.4]octane

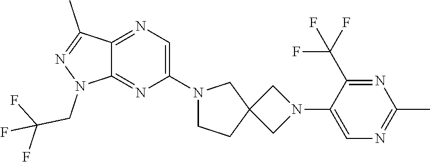 [SMILES: Cc1ncc(N2CC3(CCN(c4cnc5c(C)nn(CC(F)(F)F)c5n4)C3)C2)c(C(F)(F)F)n1]

Followed the General procedure A to afford the desired product as a white solid (9.3 mg, 24.62%). ¹H NMR (500 MHz, CDCl₃) δ 8.10 (s, 1H), 7.90 (s, 1H), 4.82 (q, J=8.5 Hz, 2H), 4.21-4.10 (m, 4H), 3.88 (s, 2H), 3.74 (t, J=6.9 Hz, 2H), 2.68 (s, 3H), 2.57 (s, 3H), 2.40 (t, J=6.9 Hz, 2H). MS m/z: 487 [M+H]⁺.

### Example 607: 6-[3-methyl-1-(2,2,2-trifluoroethyl)-1H-pyrazolo[3,4-b]pyrazin-6-yl]-2-[6-methyl-2-(trifluoromethyl)pyrimidin-4-yl]-2,6-diazaspiro[3.4]octane

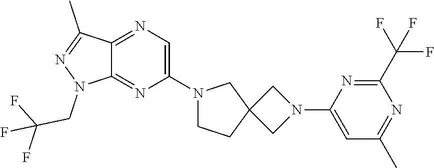 [SMILES: Cc1cc(N2CC3(CCN(c4cnc5c(C)nn(CC(F)(F)F)c5n4)C3)C2)nc(C(F)(F)F)n1]

Followed the General procedure A to afford the desired product as a white solid (14 mg, 37.06%). ¹H NMR (500 MHz, CDCl₃) δ 7.90 (s, 1H), 6.15 (s, 1H), 4.82 (q, J=8.5 Hz, 2H), 4.22-4.09 (m, 4H), 3.87 (s, 2H), 3.74 (t, J=6.9 Hz, 2H), 2.57 (s, 3H), 2.45-2.43 (m, 3H), 2.38 (t, J=6.9 Hz, 2H). MS m/z: 487 [M+H]⁺.

### Example 608: 6-[3-methyl-1-(2,2,2-trifluoroethyl)-1H-pyrazolo[3,4-b]pyrazin-6-yl]-2-[2-methyl-6-(trifluoromethyl)pyrimidin-4-yl]-2,6-diazaspiro[3.4]octane

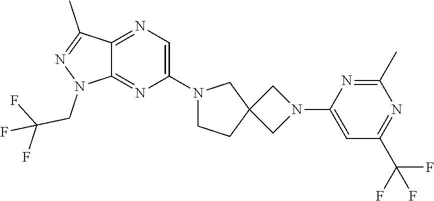 [SMILES: Cc1nc(N2CC3(CCN(c4cnc5c(C)nn(CC(F)(F)F)c5n4)C3)C2)cc(C(F)(F)F)n1]

Followed the General procedure A to afford the desired product as a white solid (14.9 mg, 39.44%). ¹H NMR (500 MHz, CDCl₃) δ 7.90 (s, 1H), 6.35 (d, J=2.2 Hz, 1H), 4.82 (q, J=8.5 Hz, 2H), 4.26-4.10 (m, 4H), 3.88 (s, 2H), 3.74 (t, J=6.9 Hz, 2H), 2.57 (d, J=7.0 Hz, 7H), 2.39 (t, J=6.9 Hz, 2H). MS m/z: 487 [M+H]⁺.

### Example 609: 2-[3-methyl-1-(2,2,2-trifluoroethyl)-1H-pyrazolo[3,4-b]pyrazin-6-yl]-6-[2-methyl-6-(trifluoromethyl)pyrimidin-4-yl]-2,6-diazaspiro[3.4]octane

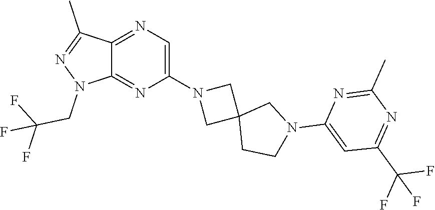 [SMILES: Cc1nc(N2CCC3(C2)CN(c2cnc4c(C)nn(CC(F)(F)F)c4n2)C3)cc(C(F)(F)F)n1]

Followed the General procedure A to afford the desired product as a white solid (15.4 mg, 40.77%). ¹H NMR (500 MHz, CDCl₃) δ 7.75 (s, 1H), 6.46 (s, 1H), 4.81 (q, J=8.5 Hz, 2H), 4.26-4.15 (m, 4H), 3.97 (br, 1H), 3.81 (br, 1H), 3.70 (br, 1H), 3.55 (br, 1H), 2.57 (d, J=8.5 Hz, 6H), 2.45-2.30 (m, 2H). MS m/z: 487 [M+H]⁺.

### Example 610: 7-[3-methyl-1-(2,2,2-trifluoroethyl)-1H-pyrazolo[3,4-b]pyrazin-6-yl]-2-[2-methyl-6-(trifluoromethyl)pyrimidin-4-yl]-2,7-diazaspiro[3.5]nonane

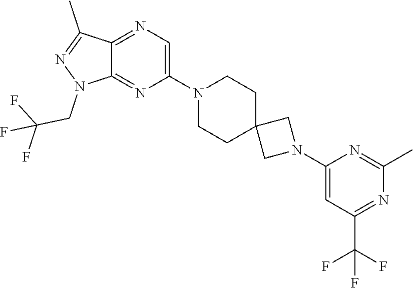 [SMILES: Cc1nc(N2CC3(CCN(c4cnc5c(C)nn(CC(F)(F)F)c5n4)CC3)C2)cc(C(F)(F)F)n1]

Followed the General procedure A to afford the desired product as a white solid (13.1 mg, 33.71%). ¹H NMR (500 MHz, CDCl₃) δ 8.20 (s, 1H), 6.35 (s, 1H), 4.82 (q, J=8.4 Hz, 2H), 4.07-3.66 (m, 8H), 2.57 (d, J=12.1 Hz, 6H), 1.98 (t, J=5.8 Hz, 4H). MS m/z: 501 [M+H]⁺.

### Example 611: 8-[3-methyl-1-(2,2,2-trifluoroethyl)-1H-pyrazolo[3,4-b]pyrazin-6-yl]-2-[2-methyl-6-(trifluoromethyl)pyrimidin-4-yl]-2,8-diazaspiro[4.5]decane

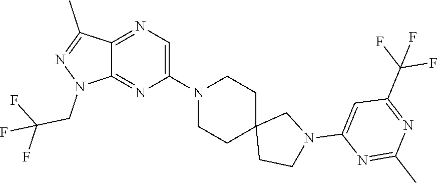 [SMILES: Cc1nc(N2CCC3(CCN(c4cnc5c(C)nn(CC(F)(F)F)c5n4)CC3)C2)cc(C(F)(F)F)n1]

Followed the General procedure D to afford the desired product as a white solid (18.7 mg, 50.2%). ¹H NMR (500 MHz, CDCl₃) δ 8.19 (s, 1H), 6.45 (d, J=7.9 Hz, 1H), 4.81 (q, J=8.5 Hz, 2H), 4.00-3.26 (m, 8H), 2.59 (s, 3H), 2.56 (s, 3H), 2.07-1.95 (m, 2H), 1.81-1.72 (m, 4H). MS m/z: 515 [M+H]⁺.

### Example 612: 6-[1-(2,2-difluoroethyl)-3-methyl-1H-pyrazolo[4,3-c]pyridin-6-yl]-2-[2-methyl-6-(trifluoromethyl)pyrimidin-4-yl]-2,6-diazaspiro[3.4]octane

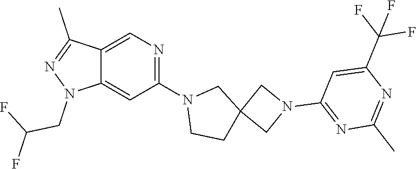 [SMILES: Cc1nc(N2CC3(CCN(c4cc5c(cn4)c(C)nn5CC(F)F)C3)C2)cc(C(F)(F)F)n1]

Followed the General procedure D to afford the desired product as a white solid (4.6 mg, 12.67%). ¹H NMR (500 MHz, CDCl₃) δ 8.44 (s, 1H), 6.33 (d, J=5.2 Hz, 1H), 6.14 (tt, d=55.4, 5.4 Hz, 1H), 6.09 (br, 1H), 4.51 (td, J=13.0, 3.9 Hz, 2H), 4.17 (d, J=33.2 Hz, 4H), 3.99-3.55 (m, 4H), 2.58 (s, 3H), 2.54 (s, 3H), 244-2.39 (m, 2H). MS m/z: 468 [M+H]⁺.

### Example 613: 7-[1-(2,2-difluoroethyl)-3-methyl-1H-pyrazolo[4,3-c]pyridin-6-yl]-2-[2-methyl-6-(trifluoromethyl)pyrimidin-4-yl]-2,7-diazaspiro[3.5]nonane

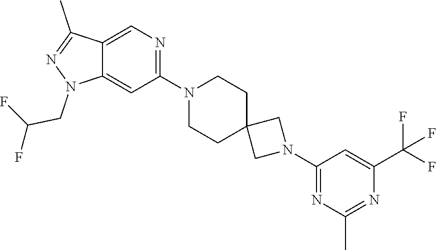 [SMILES: Cc1nc(N2CC3(CCN(c4cc5c(cn4)c(C)nn5CC(F)F)CC3)C2)cc(C(F)(F)F)n1]

Followed the General procedure D to afford the desired product as a white solid (7.8 mg, 21.62%). ¹H NMR (500 MHz, CDCl₃) δ 8.64 (s, 1H), 6.36 (s, 1H), 6.34 (s, 1H), 6.12 (tt, J=55.5, 4.3 Hz, 1H), 4.50 (td, J=13.6, 4.3 Hz, 2H), 3.78 (dd, J=151.2, 51.4 Hz, 8H), 2.55 (d, J=17.9 Hz, 6H), 1.98 (t, J=4.5 Hz, 4H). MS m/z: 482 [M+H]⁺.

### Example 614: 8-[1-(2,2-difluoroethyl)-3-methyl-1H-pyrazolo[4,3-c]pyridin-6-yl]-2-[2-methyl-6-(trifluoromethyl)pyrimidin-4-yl]-2,8-diazaspiro[4.5]decan-3-one

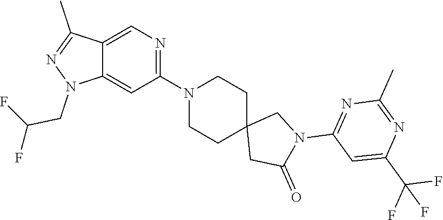 [SMILES: Cc1nc(N2CC3(CCN(c4cc5c(cn4)c(C)nn5CC(F)F)CC3)CC2=O)cc(C(F)(F)F)n1]

Followed the General procedure D to afford the desired product as a white solid (3.7 mg, 10.37%). ¹H NMR (500 MHz, CDCl₃) δ 8.69 (br, 1H), 8.58 (d, J=4.7 Hz, 1H), 6.39 (s, 1H), 6.13 (tt, J=55.4, 4.3 Hz, 1H), 4.52 (t, J=12.7 Hz, 2H), 4.01 (d, J=9.0 Hz, 2H), 3.82 (s, 2H), 3.61 (s, 2H), 2.72 (s, 3H), 2.70 (s, 2H), 2.55 (s, 3H), 1.94-1.87 (m, 4H). MS m/z: 510 [M+H]⁺.

### Example 615: 2-[3-methyl-1-(2,2,2-trifluoroethyl)-1H-pyrazolo[3,4-d]pyrimidin-6-yl]-7-[2-(trifluoromethyl)pyrimidin-5-yl]-2,7-diazaspiro[4.4]nonane

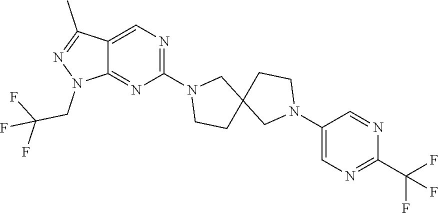 [SMILES: Cc1nn(CC(F)(F)F)c2nc(N3CCC4(CCN(c5cnc(C(F)(F)F)nc5)C4)C3)ncc12]

Followed the General procedure A to afford the desired product as a white solid (20 mg, 37.32%). ¹H NMR (500 MHz, CDCl₃) δ 8.72 (s, 1H), 8.10 (s, 2H), 4.78 (s, 2H), 3.96-3.65 (m, 4H), 3.59 (t, J=6.9 Hz, 2H), 3.51-3.36 (m, 2H), 2.51 (s, 3H), 2.25-2.08 (m, 4H). MS m/z: 487 [M+H]⁺.

### Example 616: 2-[3-methyl-1-(2,2,2-trifluoroethyl)-1H-pyrazolo[3,4-d]pyrimidin-6-yl]-7-[2-methyl-6-(trifluoromethyl)pyrimidin-4-yl]-2,7-diazaspiro[4.4]nonane

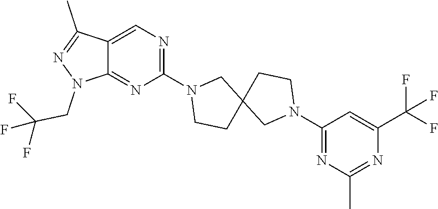 [SMILES: Cc1nc(N2CCC3(CCN(c4ncc5c(C)nn(CC(F)(F)F)c5n4)C3)C2)cc(C(F)(F)F)n1]

Followed the General procedure A to afford the desired product as a white solid (22.3 mg, 42.53%). ¹H NMR (500 MHz, CDCl₃) δ 8.68 (br, 1H), 6.44 (d, J=31.2 Hz, 1H), 4.78 (s, 2H), 3.89-3.34 (m, 8H), 2.60-2.54 (m, 3H), 2.50 (s, 3H), 2.25-2.05 (m, 4H). MS m/z: 501 [M+H]⁺.

### Example 617: 2-[3-methyl-1-(2,2,2-trifluoroethyl)-1H-pyrazolo[3,4-d]pyrimidin-6-yl]-6-[2-methyl-6-(trifluoromethyl)pyrimidin-4-yl]-2,6-diazaspiro[3.4]octane

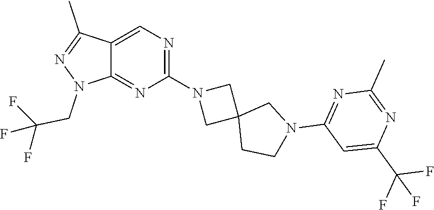 [SMILES: Cc1nc(N2CCC3(C2)CN(c2ncc4c(C)nn(CC(F)(F)F)c4n2)C3)cc(C(F)(F)F)n1]

Followed the General procedure A to afford the desired product as a white solid (17.1 mg, 31.91%). ¹H NMR (500 MHz, CDCl₃) δ 8.69 (s, 1H), 6.46-6.44 (m, 1H), 4.77 (q, J=8.4 Hz, 2H), 4.23-4.19 (m, 4H), 3.93 (s, 1H), 3.79 (t, J=5.5 Hz, 1H), 3.66 (s, 1H), 3.53 (t, J=5.5 Hz, 1H), 2.57 (s, 3H), 2.51 (s, 3H), 2.43-2.22 (m, 2H). MS m/z: 487 [M+H]⁺.

### Example 618: 7-[3-methyl-1-(2,2,2-trifluoroethyl)-1H-pyrazolo[3,4-d]pyrimidin-6-yl]-2-[2-methyl-6-(trifluoromethyl)pyrimidin-4-yl]-2,7-diazaspiro[3.5]nonane

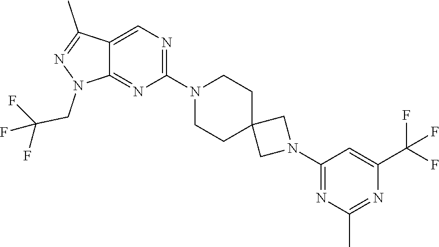 [SMILES: Cc1nc(N2CC3(CCN(c4ncc5c(C)nn(CC(F)(F)F)c5n4)CC3)C2)cc(C(F)(F)F)n1]

Followed the General procedure A to afford the desired product as a white solid (17.2 mg, 32.8%). ¹H NMR (500 MHz, CDCl₃) δ 8.67 (s, 1H), 6.34 (s, 1H), 4.77 (q, J=8.5 Hz, 2H), 4.10-3.77 (m, 8H), 2.57 (s, 3H), 2.49 (s, 3H), 1.90 (t, J=5.8 Hz, 4H). MS m/z: 501 [M+H]⁺.

### Example 619: 8-[3-methyl-1-(2,2,2-trifluoroethyl)-1H-pyrazolo[3,4-d]pyrimidin-6-yl]-2-[2-methyl-6-(trifluoromethyl)pyrimidin-4-yl]-2,8-diazaspiro[4.5]decane

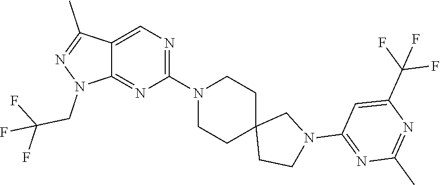 [SMILES: Cc1nc(N2CCC3(CCN(c4ncc5c(C)nn(CC(F)(F)F)c5n4)CC3)C2)cc(C(F)(F)F)n1]

Followed the General procedure A to afford the desired product as a white solid (18.9 mg, 36.78%). ¹H NMR (500 MHz, CDCl₃) δ 8.68 (s, 1H), 6.45-6.43 (m, 1H), 4.77 (q, J=8.5 Hz, 2H), 4.15-4.10 (m, 1H), 4.03-3.92 (m, 2H), 3.89-3.74 (m, 2H), 3.64 (s, 1H), 3.51 (t, J=7.1 Hz, 1H), 3.30 (s, 1H), 2.58 (s, 3H), 2.50 (s, 3H), 2.07-1.93 (m, 2H), 1.72-1.67 (m, 4H). MS m/z: 515 [M+H]⁺.

### Example 620: 6-[3-methyl-1-(2,2,2-trifluoroethyl)-1H-pyrazolo[3,4-d]pyrimidin-6-yl]-2-[2-methyl-4-(trifluoromethyl)pyrimidin-5-yl]-2,6-diazaspiro[3.4]octane

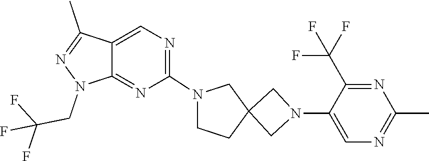 [SMILES: Cc1ncc(N2CC3(CCN(c4ncc5c(C)nn(CC(F)(F)F)c5n4)C3)C2)c(C(F)(F)F)n1]

Followed the General procedure A to afford the desired product as a white solid (6.7 mg, 12.5%). ¹H NMR (500 MHz, CDCl₃) δ 8.72 (s, 1H), 8.09 (s, 1H), 4.78 (q, J=8.4 Hz, 2H), 4.19-4.07 (m, 4H), 4.00-3.71 (m, 4H), 2.67 (s, 3H), 2.51 (s, 3H), 2.34 (t, J=7.0 Hz, 2H). MS m/z: 487 [M+H]⁺.

### Example 621: 6-[1-(2,2-difluoroethyl)-1H-pyrazolo[3,4-b]pyridin-6-yl]-2-[2-methyl-6-(trifluoromethyl)pyrimidin-4-yl]-2,6-diazaspiro[3.4]octane

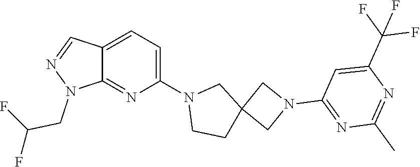 [SMILES: Cc1nc(N2CC3(CCN(c4ccc5cnn(CC(F)F)c5n4)C3)C2)cc(C(F)(F)F)n1]

Followed the General procedure A to afford the desired product as a white solid (14.5 mg, 30.52%). ¹H NMR (500 MHz, CDCl₃) δ 7.81 (s, 1H), 7.77 (d, J=8.8 Hz, 1H), 6.34 (t, J=4.4 Hz, 2H), 6.25 (td, J=55.6, 4.5 Hz, 1H), 4.70 (td, J=13.4, 4.6 Hz, 2H), 4.27-4.06 (m, 4H), 3.84 (s, 2H), 3.67 (t, J=6.9 Hz, 2H), 2.57 (s, 3H), 2.35 (t, J=6.9 Hz, 2H). MS m/z: 454 [M+H]⁺.

### Example 622: 6-[1-(2,2-difluoroethyl)-3-methyl-1H-pyrazolo[3,4-b]pyridin-6-yl]-2-[2-methyl-6-(trifluoromethyl)pyrimidin-4-yl]-2,6-diazaspiro[3.4]octane

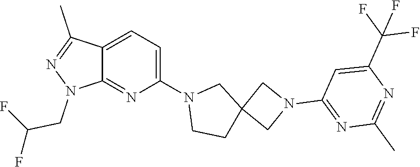 [SMILES: Cc1nc(N2CC3(CCN(c4ccc5c(C)nn(CC(F)F)c5n4)C3)C2)cc(C(F)(F)F)n1]

Followed the General procedure A to afford the desired product as a white solid (8.3 mg, 16.95%). ¹H NMR (500 MHz, CDCl₃) δ 7.70 (d, J=8.8 Hz, 1H), 6.34 (s, 1H), 6.28 (d, J=8.7 Hz, 1H), 6.22 (tt, J=55.7, 4.5 Hz, 1H)), 4.62 (td, J=13.4, 4.6 Hz, 2H), 4.25-4.06 (m, 4H), 3.83 (s, 2H), 3.66 (t, J=6.9 Hz, 2H), 2.57 (s, 3H), 2.46 (s, 3H), 2.34 (t, J=6.8 Hz, 2H). MS m/z: 468 [M+H]⁺.

### Example 623: 7-[1-(2,2-difluoroethyl)-1H-pyrazolo[3,4-b]pyridin-6-yl]-2-[2-methyl-6-(trifluoromethyl)pyrimidin-4-yl]-2,7-diazaspiro[3.5]nonane

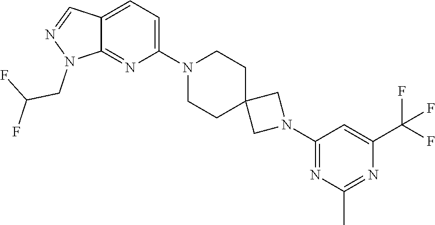 [SMILES: Cc1nc(N2CC3(CCN(c4ccc5cnn(CC(F)F)c5n4)CC3)C2)cc(C(F)(F)F)n1]

Followed the General procedure A to afford the desired product as a white solid (4 mg, 8%). MS m/z: 468 [M+H]⁺.

### Example 624: 7-[1-(2,2-difluoroethyl)-3-methyl-1H-pyrazolo[3,4-b]pyridin-6-yl]-2-[2-methyl-6-(trifluoromethyl)pyrimidin-4-yl]-2,7-diazaspiro[3.5]nonane

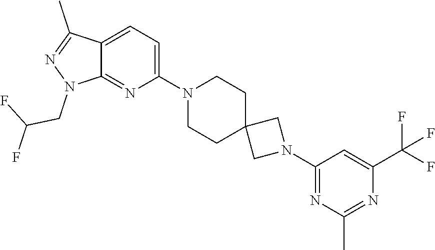 [SMILES: Cc1nc(N2CC3(CCN(c4ccc5c(C)nn(CC(F)F)c5n4)CC3)C2)cc(C(F)(F)F)n1]

Followed the General procedure A to afford the desired product as a white solid (2 mg, 4%). MS m/z: 482 [M+H]⁺.

### Example 625: 8-[1-(2,2-difluoroethyl)-1H-pyrazolo[3,4-b]pyridin-6-yl]-2-[2-methyl-6-(trifluoromethyl)pyrimidin-4-yl]-2,8-diazaspiro[4.5]decane

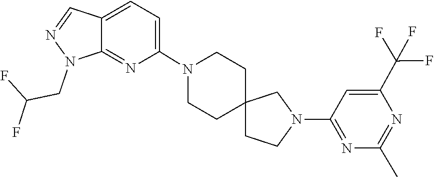 [SMILES: Cc1nc(N2CCC3(CCN(c4ccc5cnn(CC(F)F)c5n4)CC3)C2)cc(C(F)(F)F)n1]

Followed the General procedure A to afford the desired product as a white solid (10.1 mg, 20.02%). ¹H NMR (500 MHz, CDCl₃) δ 7.80 (s, 1H), 7.78 (d, J=9.1 Hz, 1H), 6.65 (d, J=9.0 Hz, 1H), 6.44 (d, J=8.1 Hz, 1H), 6.24 (tt, J=55.8, 4.6 Hz, 1H), 4.70 (td, J=13.4, 4.5 Hz, 2H), 3.93-3.68 (m, 4H), 3.64 (s, 2H), 3.51 (t, J=7.1 Hz, 1H), 3.30 (s, 1H), 2.57 (s, 3H), 2.06-1.92 (m, 2H), 1.73 (td, J=10.9, 4.9 Hz, 4H). MS m/z: 482 [M+H]⁺.

### Example 626: 8-[1-(2,2-difluoroethyl)-3-methyl-1H-pyrazolo[3,4-b]pyridin-6-yl]-2-[2-methyl-6-(trifluoromethyl)pyrimidin-4-yl]-2,8-diazaspiro[4.5]decane

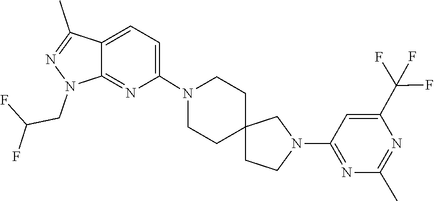 [SMILES: Cc1nc(N2CCC3(CCN(c4ccc5c(C)nn(CC(F)F)c5n4)CC3)C2)cc(C(F)(F)F)n1]

Followed the General procedure A to afford the desired product as a white solid (4.1 mg, 8%). MS m/z: 496 [M+H]⁺.

### Example 627: 8-[1-(2,2-difluoroethyl)-1H-pyrazolo[3,4-b]pyridin-6-yl]-2-[2-methyl-6-(trifluoromethyl)pyrimidin-4-yl]-2,8-diazaspiro[4.5]decan-3-one

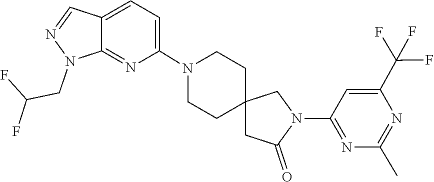 [SMILES: Cc1nc(N2CC3(CCN(c4ccc5cnn(CC(F)F)c5n4)CC3)CC2=O)cc(C(F)(F)F)n1]

Followed the General procedure A to afford the desired product as a white solid (4.4 mg, 8%). MS m/z: 496 [M+H]⁺.

### Example 628: 8-[1-(2,2-difluoroethyl)-3-methyl-1H-pyrazolo[3,4-b]pyridin-6-yl]-2-[2-methyl-6-(trifluoromethyl)pyrimidin-4-yl]-2,8-diazaspiro[4.5]decan-3-one

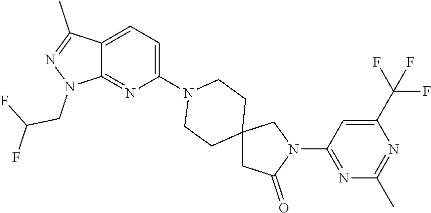 [SMILES: Cc1nc(N2CC3(CCN(c4ccc5c(C)nn(CC(F)F)c5n4)CC3)CC2=O)cc(C(F)(F)F)n1]

Followed the General procedure A to afford the desired product as a white solid (3.6 mg, 7%). MS m/z: 510 [M+H]⁺.

### Example 629: 6-{1-tert-butyl-1H-pyrazolo[3,4-d]pyrimidin-6-yl}-2-[2-methyl-6-(trifluoromethyl)pyrimidin-4-yl]-2,6-diazaspiro[3.4]octane

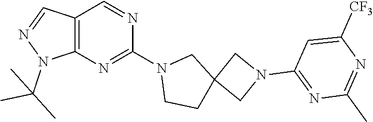 [SMILES: Cc1nc(N2CC3(CCN(c4ncc5cnn(C(C)(C)C)c5n4)C3)C2)cc(C(F)(F)F)n1]

Followed the General procedure A to afford the desired product as a white solid (29 mg, 62.2%). ¹H NMR (500 MHz, CDCl₃) δ 8.76 (s, 1H), 7.80 (s, 1H), 6.34 (s, 1H), 4.13 (dd, J=17.8, 10.7 Hz, 4H), 3.84 (d, J=62.2 Hz, 4H), 2.57 (s, 3H), 2.32 (t, J=6.9 Hz, 2H), 1.77 (s, 9H). MS m/z: 447 [M+H]⁺.

### Example 630: 8-[1-(2,2-difluoroethyl)-3-methyl-1H-pyrazolo[4,3-c]pyridin-6-yl]-2-[2-methyl-6-(trifluoromethyl)pyrimidin-4-yl]-2,8-diazaspiro[4.5]decane

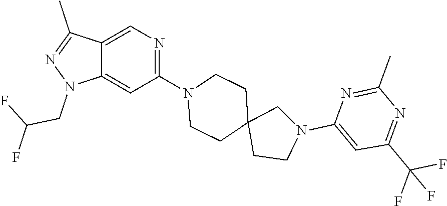 [SMILES: Cc1nc(N2CCC3(CCN(c4cc5c(cn4)c(C)nn5CC(F)F)CC3)C2)cc(C(F)(F)F)n1]

Followed the General procedure D to afford the desired product as a white solid (5.7 mg, 11.52%). ¹H NMR (500 MHz, CDCl₃) δ 8.65 (br, 1H), 6.47-6.40 (m, 1H), 6.35 (s, 1H), 6.12 (tt, J=55.5, 4.3 Hz, 1H), 4.50 (td, J=13.5, 4.3 Hz, 2H), 3.88-3.31 (m, 8H), 2.57 (s, 3H), 2.54 (s, 3H), 2.05-1.96 (m, 2H), 1.86-1.75 (m, 4H). MS m/z: 496 [M+H]⁺.

### Example 631: 6-[1-(2,2-difluoroethyl)-1H-pyrazolo[4,3-c]pyridin-6-yl]-2-[2-methyl-6-(trifluoromethyl)pyrimidin-4-yl]-2,6-diazaspiro[3.4]octane

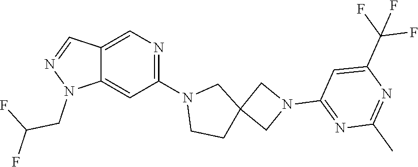 [SMILES: Cc1nc(N2CC3(CCN(c4cc5c(cn4)cnn5CC(F)F)C3)C2)cc(C(F)(F)F)n1]

Followed the General procedure D to afford the desired product as a light blue solid (18.6 mg, 37.23%). ¹H NMR (500 MHz, CDCl₃) δ 8.70 (s, 1H), 8.00 (s, 1H), 6.34 (s, 1H), 6.14 (tt, J=55.4, 4.3 Hz, 1H), 6.06 (d, J=13.8 Hz, 1H), 4.57 (td, J=13.5, 4.3 Hz, 2H), 4.27-4.00 (m, 4H), 3.84 (s, 2H), 3.65 (t, J=6.8 Hz, 2H), 2.57 (s, 3H), 2.36 (t, J=6.8 Hz, 2H). MS m/z: 454 [M+H]⁺.

### Example 632: 7-[1-(2,2-difluoroethyl)-1H-pyrazolo[4,3-c]pyridin-6-yl]-2-[2-methyl-6-(trifluoromethyl)pyrimidin-4-yl]-2,7-diazaspiro[3.5]nonane

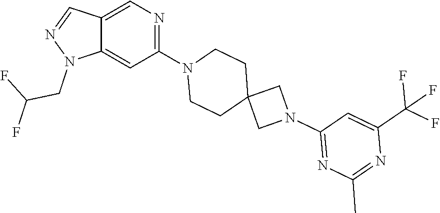 [SMILES: Cc1nc(N2CC3(CCN(c4cc5c(cn4)cnn5CC(F)F)CC3)C2)cc(C(F)(F)F)n1]

Followed the General procedure D to afford the desired product as a white solid (9.6 mg, 19.6%). ¹H NMR (500 MHz, CDCl₃) δ 8.73 (s, 1H), 8.00 (s, 1H), 6.42 (s, 1H), 6.34 (s, 1H), 6.14 (tt, J=55.4, 4.3 Hz, 1H), 4.59 (td, J=13.6, 4.3 Hz, 2H), 4.00-3.58 (m, 8H), 2.57 (s, 3H), 1.98 (t, J=5.6 Hz, 4H). MS m/z: 468 [M+H]⁺.

### Example 633: 8-[1-(2,2-difluoroethyl)-1H-pyrazolo[4,3-c]pyridin-6-yl]-2-[2-methyl-6-(trifluoromethyl)pyrimidin-4-yl]-2,8-diazaspiro[4.5]decane

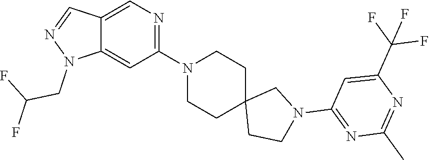 [SMILES: Cc1nc(N2CCC3(CCN(c4cc5c(cn4)cnn5CC(F)F)CC3)C2)cc(C(F)(F)F)n1]

Followed the General procedure A to afford the desired product as a white solid (13.7 mg, 28.49%). ¹H NMR (500 MHz, CDCl₃) δ 8.73 (s, 1H), 8.00 (s, 1H), 6.44-6.43 (m, 1H), 6.41 (s, 1H), 6.14 (tt, J=55.4, 4.3 Hz, 1H), 4.59 (td, J=13.6, 4.3 Hz, 2H), 3.86-3.30 (m, 8H), 2.57 (s, 3H), 2.00 (dt, J=29.5, 7.1 Hz, 2H), 1.85-1.75 (m, 4H). MS m/z: 482 [M+H]⁺.

### Example 634: 8-[1-(2,2-difluoroethyl)-1H-pyrazolo[4,3-c]pyridin-6-yl]-2-[2-methyl-6-(trifluoromethyl)pyrimidin-4-yl]-2,8-diazaspiro[4.5]decan-3-one

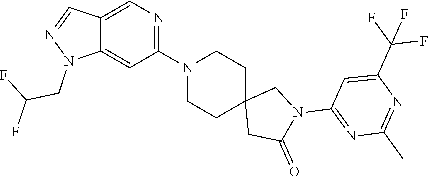 [SMILES: Cc1nc(N2CC3(CCN(c4cc5c(cn4)cnn5CC(F)F)CC3)CC2=O)cc(C(F)(F)F)n1]

Followed the General procedure D to afford the desired product as a white solid (4.6 mg, 9.73%). ¹H NMR (500 MHz, CDCl₃) δ 8.74 (s, 1H), 8.58 (s, 1H), 8.01 (s, 1H), 6.43 (s, 1H), 6.15 (tt, J=55.4, 4.3 Hz, 1H), 4.60 (td, J=13.6, 4.3 Hz, 2H), 4.00 (s, 2H), 3.86-3.72 (m, 2H), 3.60-3.55 (m, 2H), 2.72 (s, 3H), 2.69 (s, 2H), 1.88 (d, J=11.4 Hz, 4H). MS m/z: 496 [M+H]⁺.

### Example 635: 6-[1-(2,2-difluoroethyl)-1H-pyrazolo[4,3-b]pyridin-6-yl]-2-[2-(trifluoromethyl)pyridin-4-yl]-2,6-diazaspiro[3.4]octane

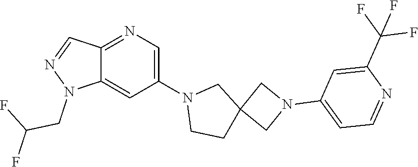 [SMILES: FC(F)Cn1ncc2ncc(N3CCC4(C3)CN(c3ccnc(C(F)(F)F)c3)C4)cc21]

Followed the General procedure D to afford the desired product as a white solid (28.4 mg, 80.17%). ¹H NMR (500 MHz, CDCl₃) δ 8.33 (d, J=5.6 Hz, 1H), 8.15 (J=2.4 Hz, 1H), 8.13 (d J=1.7 Hz, 1H), 6.63 (d, J=2.3 Hz, 1H), 6.60 (d J=1.9 Hz, 1H), 6.39 (dd, J=5.7, 2.3 Hz, 1H), 6.13 (tt, J=55.5, 4.3 Hz, 1H), 4.61 (td, J=13.6, 4.3 Hz, 2H), 4.12-4.01 (m, 4H), 3.71 (s, 2H), 3.58 (t, J=6.8 Hz, 2H), 2.41 (t, J=6.8 Hz, 2H). MS m/z: 439 [M+H]⁺.

### Example 636: 6-[1-(2,2-difluoroethyl)-1H-pyrazolo[4,3-b]pyridin-6-yl]-2-[2-methyl-6-(trifluoromethyl)pyrimidin-4-yl]-2,6-diazaspiro[3.4]octane

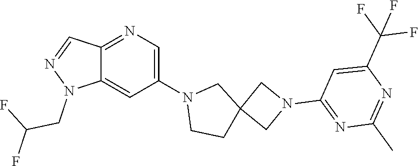 [SMILES: Cc1nc(N2CC3(CCN(c4cnc5cnn(CC(F)F)c5c4)C3)C2)cc(C(F)(F)F)n1]

Followed the General procedure D to afford the desired product as a white solid (12.2 mg, 34.65%). ¹H NMR (500 MHz, CDCl₃) δ 8.15 (d, J=7.0 Hz, 1H), 8.14 (d, J=7.0 Hz, 1H), 6.62 (d, J=2.4 Hz, 1H), 6.35 (s, 1H), 6.13 (tt, J=55.5, 4.3 Hz, 1H), 4.62 (td, J=13.6, 4.3 Hz, 2H), 4.27-4.07 (m, 4H), 3.70 (s, 2H), 3.58 (t, J=6.8 Hz, 2H), 2.58 (s, 3H), 2.40 (t, J=6.8 Hz, 2H). MS m/z: 454 [M+H]⁺.

### Example 637: 6-{imidazo[1,2-b]pyridazin-6-yl}-2-[2-methyl-6-(trifluoromethyl)pyrimidin-4-yl]-2,6-diazaspiro[3.4]octane

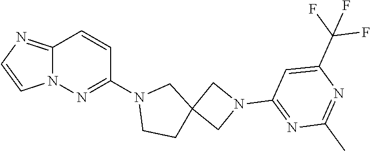 [SMILES: Cc1nc(N2CC3(CCN(c4ccc5nccn5n4)C3)C2)cc(C(F)(F)F)n1]

Followed the General procedure A to afford the desired product as a white solid (12.2 mg, 28.44%). ¹H NMR (500 MHz, CDCl₃) δ 7.81 (d, J=9.8 Hz, 1H), 7.68 (s, 1H), 7.57-7.52 (m, 1H), 6.62 (d, J=9.8 Hz, 1H), 6.34 (s, 1H), 4.21-4.07 (m, 4H), 3.77 (s, 2H), 3.63 (t, J=6.9 Hz, 2H), 2.57 (s, 3H), 2.36 (t, J=6.9 Hz, 2H). MS m/z: 390 [M+H]⁺.

### Example 638: 2-[2-methyl-6-(trifluoromethyl)pyrimidin-4-yl]-6-{2-methylimidazo[1,2-b]pyridazin-6-yl}-2,6-diazaspiro[3.4]octane

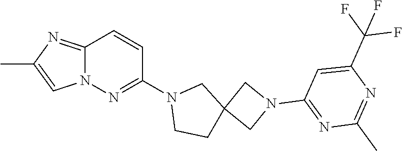 [SMILES: Cc1cn2nc(N3CCC4(CN(c5cc(C(F)(F)F)nc(C)n5)C4)C3)ccc2n1]

Followed the General procedure A to afford the desired product as a white solid (11.6 mg, 26.1%). ¹H NMR (500 MHz, CDCl₃) δ 7.74-7.65 (m, 1H), 7.44 (t, J=0.8 Hz, 1H), 6.53 (d, J=9.7 Hz, 1H), 6.34 (s, 1H), 4.25-4.08 (m, 4H), 3.74 (s, 2H), 3.61 (t, J=6.9 Hz, 2H), 2.57 (s, 3H), 2.43 (d, J=0.9 Hz, 3H), 2.34 (t, J=6.9 Hz, 2H). MS m/z: 404 [M+H]⁺.

### Example 639: 6-[1-(2,2-difluoroethyl)-1H-pyrazolo[3,4-b]pyrazin-6-yl]-2-(2,6-dimethylpyrimidin-4-yl)-2,6-diazaspiro[3.4]octane

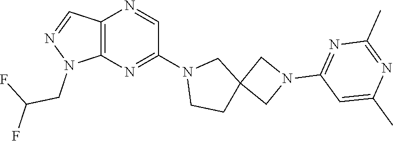 [SMILES: Cc1cc(N2CC3(CCN(c4cnc5cnn(CC(F)F)c5n4)C3)C2)nc(C)n1]

Followed the General procedure A to afford the desired product as a white solid (14.3 mg, 39.56%). ¹H NMR (500 MHz, CDCl₃) δ 8.06 (s, 1H), 7.96 (s, 1H), 6.22 (tt, J=55.7, 4.5 Hz, 1H), 5.92 (s, 1H), 4.66 (td, J=13.4, 4.5 Hz, 2H), 4.18-4.03 (m, 4H), 3.87 (s, 2H), 3.74 (t, J=6.9 Hz, 2H), 2.53 (s, 3H), 2.38-2.36 (m, 5H). MS m/z: 401 [M+H]⁺.

### Example 640: 6-[1-(2,2-difluoroethyl)-3-methyl-1H-pyrazolo[3,4-b]pyrazin-6-yl]-2-(2,6-dimethylpyrimidin-4-yl)-2,6-diazaspiro[3.4]octane

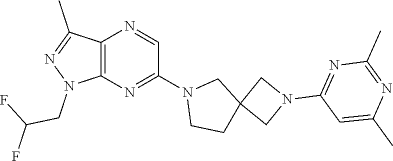 [SMILES: Cc1cc(N2CC3(CCN(c4cnc5c(C)nn(CC(F)F)c5n4)C3)C2)nc(C)n1]

Followed the General procedure A to afford the desired product as a white solid (13.8 mg, 36.88%). ¹H NMR (500 MHz, CDCl₃) δ 7.89 (s, 1H), 6.20 (tt, J=55.7, 4.5 Hz, 1H), 5.92 (s, 1H), 4.59 (td, J=13.4, 4.5 Hz, 2H), 4.15 (d, J=17.4 Hz, 4H), 3.87 (s, 2H), 3.74 (t, J=6.8 Hz, 2H), 2.59 (s, 3H), 2.55 (s, 3H), 2.48-2.35 (m, 5H). MS m/z: 415 [M+H]⁺.

### Example 641: 6-[1-(2,2-difluoroethyl)-3-methyl-1H-pyrazolo[3,4-d]pyrimidin-6-yl]-2-(2,6-dimethylpyrimidin-4-yl)-2,6-diazaspiro[3.4]octane

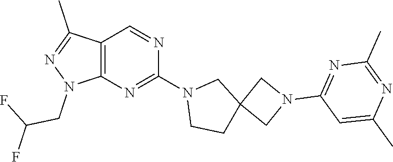 [SMILES: Cc1cc(N2CC3(CCN(c4ncc5c(C)nn(CC(F)F)c5n4)C3)C2)nc(C)n1]

Followed the General procedure A to afford the desired product as a white solid (12.8 mg, 34.21%). ¹H NMR (500 MHz, CDCl₃) δ 8.67 (s, 1H), 6.19 (tt, J=55.7, 4.5 Hz, 1H), 5.90 (s, 1H), 4.55 (td, J=13.4, 4.5 Hz, 2H), 4.12-4.04 (m, 4H), 3.89 (s, 2H), 3.76 (s, 2H), 2.53 (s, 3H), 2.49 (s, 3H), 2.36 (s, 3H), 2.29 (t, J=6.9 Hz, 2H). MS m/z: 415 [M+H]⁺.

### Example 642: 6-[1-(2,2-difluoroethyl)-1H-pyrazolo[3,4-d]pyrimidin-6-yl]-2-(2,6-dimethylpyrimidin-4-yl)-2,6-diazaspiro[3.4]octane

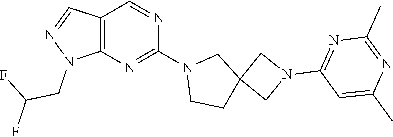 [SMILES: Cc1cc(N2CC3(CCN(c4ncc5cnn(CC(F)F)c5n4)C3)C2)nc(C)n1]

Followed the General procedure A to afford the desired product as a white solid (13.8 mg, 38.18%). ¹H NMR (500 MHz, CDCl₃) δ 8.78 (s, 1H), 7.90 (s, 1H), 6.39-6.08 (m, 1H), 5.91 (s, 1H), 4.64 (td, J=13.4, 4.5 Hz, 2H), 4.12-4.05 (m, 4H), 3.90 (s, 2H), 3.77 (s, 2H), 2.53 (s, 3H), 2.37 (s, 3H), 2.30 (t, J=6.9 Hz, 2H). MS m/z: 401 [M+H]⁺.

### Example 643: 2-(2,6-dimethylpyrimidin-4-yl)-6-[1-(oxetan-3-yl)-1H-pyrazolo[3,4-d]pyrimidin-6-yl]-2,6-diazaspiro[3.4]octane

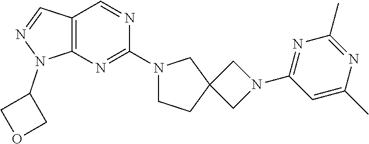 [SMILES: Cc1cc(N2CC3(CCN(c4ncc5cnn(C6COC6)c5n4)C3)C2)nc(C)n1]

Followed the General procedure A to afford the desired product as a white solid (10.8 mg, 30.48%). ¹H NMR (500 MHz, CDCl₃) δ 8.77 (s, 1H), 7.95 (d, J=0.5 Hz, 1H), 5.97-5.91 (m, 1H), 5.91 (s, 1H), 5.31 (t, J=6.6 Hz, 2H), 5.05 (dd, J=7.9, 6.6 Hz, 2H), 4.15-4.06 (m, 4H), 3.90 (s, 2H), 3.77 (t, J=7 Hz, 2H), 2.56 (s, 3H), 2.40 (s, 3H), 2.31 (t, J=6.9 Hz, 2H) MS m/z: 393 [M+H]⁺.

### Example 644: 2-(2,6-dimethylpyrimidin-4-yl)-6-[3-methyl-1-(oxetan-3-yl)-1H-pyrazolo[3,4-d]pyrimidin-6-yl]-2,6-diazaspiro[3.4]octane

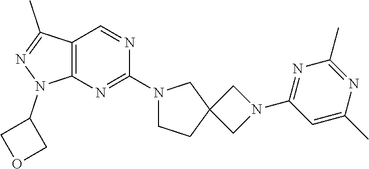 [SMILES: Cc1cc(N2CC3(CCN(c4ncc5c(C)nn(C6COC6)c5n4)C3)C2)nc(C)n1]

Followed the General procedure A to afford the desired product as a white solid (11.3 mg, 30.79%). ¹H NMR (500 MHz, CDCl₃) δ 8.66 (s, 1H), 5.90 (s, 1H), 5.89-5.83 (m, 1H), 5.28 (t, J=6.7 Hz, 2H), 5.02 (dd, J=8.0, 6.6 Hz, 2H), 4.13-4.05 (m, 4H), 3.89 (s, 2H), 3.75 (s, 2H), 2.55 (s, 3H), 2.53 (s, 3H), 2.38 (s, 3H), 2.29 (t, J=6.9 Hz, 2H). MS m/z: 407 [M+H]⁺.

### Example 645: 2-(2,6-dimethylpyrimidin-4-yl)-6-[3-methyl-1-(oxetan-3-yl)-1H-pyrazolo[3,4-b]pyrazin-6-yl]-2,6-diazaspiro[3.4]octane

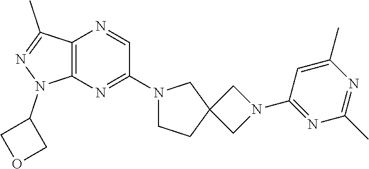 [SMILES: Cc1cc(N2CC3(CCN(c4cnc5c(C)nn(C6COC6)c5n4)C3)C2)nc(C)n1]

Followed the General procedure A to afford the desired product as a white solid (3.7 mg, 10.08%). ¹H NMR (500 MHz, CDCl₃) δ 7.87 (s, 1H), 5.92 (s, 1H), 5.90-5.84 (m, 1H), 5.33 (t, J=6.6 Hz, 2H), 5.03 (dd, J=7.9, 6.5 Hz, 2H), 4.16-4.10 (m, 4H), 3.87 (s, 2H), 3.73 (t, J=6.9 Hz, 2H), 2.58 (d, J=9.0 Hz, 5H), 2.42 (s, 3H), 2.37 (t, J=6.9 Hz, 2H). MS m/z: 407 [M+H]⁺.

### Example 646: 2-(2,6-dimethylpyrimidin-4-yl)-6-[1-(oxetan-3-yl)-1H-pyrazolo[3,4-b]pyrazin-6-yl]-2,6-diazaspiro[3.4]octane

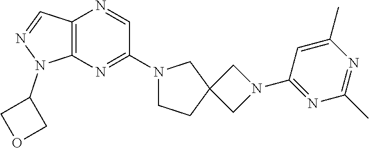 [SMILES: Cc1cc(N2CC3(CCN(c4cnc5cnn(C6COC6)c5n4)C3)C2)nc(C)n1]

Followed the General procedure A to afford the desired product as a white solid (10.5 mg, 29.64%). ¹H NMR (500 MHz, CDCl₃) δ 8.10 (s, 1H), 7.95 (s, 1H), 5.99-5.89 (m, 2H), 5.34 (t, J=6.7 Hz, 2H), 5.09-5.02 (m, 2H), 4.20-4.08 (m, 4H), 3.88 (s, 2H), 3.74 (t, J=6.9 Hz, 2H), 2.58 (s, 3H), 2.43 (s, 3H), 2.38 (t, J=6.9 Hz, 2H). MS m/z: 393 [M+H]⁺.

### Example 647: 6-{1-methyl-1H-pyrazolo[3,4-d]pyrimidin-6-yl}-2-[2-methyl-6-(trifluoromethyl)pyrimidin-4-yl]-2,6-diazaspiro[3.4]octane

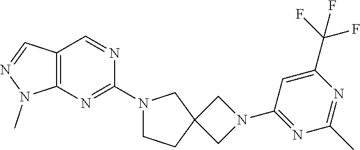 [SMILES: Cc1nc(N2CC3(CCN(c4ncc5cnn(C)c5n4)C3)C2)cc(C(F)(F)F)n1]

Followed the General procedure A to afford the desired product as a white solid (4.5 mg, 10.66%). ¹H NMR (500 MHz, CDCl₃) δ 8.76 (s, 1H), 7.85 (s, 1H), 6.33 (s, 1H), 4.25-4.04 (m, 4H), 3.92 (d, J=5.2 Hz, 5H), 3.79 (t, J=6.8 Hz, 2H), 2.57 (s, 3H), 2.32 (t, J=6.9 Hz, 2H). MS m/z: 405 [M+H]⁺.

### Example 648: 6-{1,3-dimethyl-1H-pyrazolo[3,4-d]pyrimidin-6-yl}-2-[2-methyl-6-(trifluoromethyl)pyrimidin-4-yl]-2,6-diazaspiro[3.4]octane

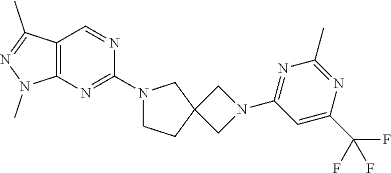 [SMILES: Cc1nc(N2CC3(CCN(c4ncc5c(C)nn(C)c5n4)C3)C2)cc(C(F)(F)F)n1]

Followed the General procedure A to afford the desired product as a white solid (12 mg, 48.44%). ¹H NMR (500 MHz, CDCl₃) δ 8.68 (s, 1H), 6.33 (s, 1H), 4.16 (s, 4H), 4.02-3.90 (m, 2H), 3.84 (s, 3H), 3.80 (s, 2H), 2.57 (s, 3H), 2.49 (s, 3H), 2.32 (t, J=6.9 Hz, 2H). MS m/z: 419 [M+H]⁺.

### Example 649: 6-{1-methyl-1H-pyrazolo[3,4-b]pyrazin-6-yl}-2-[2-methyl-6-(trifluoromethyl)pyrimidin-4-yl]-2,6-diazaspiro[3.4]octane

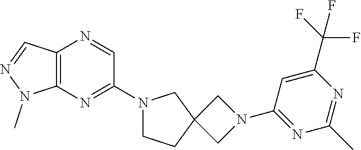 [SMILES: Cc1nc(N2CC3(CCN(c4cnc5cnn(C)c5n4)C3)C2)cc(C(F)(F)F)n1]

Followed the General procedure A to afford the desired product as a white solid (27 mg, 56%). ¹H NMR (500 MHz, CDCl₃) δ 8.00 (s, 1H), 7.93 (s, 1H), 6.35 (s, 1H), 4.18 (s, 4H), 3.97 (s, 3H), 3.90 (s, 2H), 3.75 (t, J=6.9 Hz, 2H), 2.58 (s, 3H), 2.39 (t, J=6.9 Hz, 2H). MS m/z: 405 [M+H]⁺.

### Example 650: 6-[1-(difluoromethyl)-1H-pyrazolo[3,4-d]pyrimidin-6-yl]-2-[2-methyl-6-(trifluoromethyl)pyrimidin-4-yl]-2,6-diazaspiro[3.4]octane

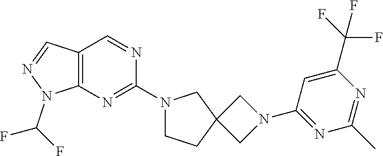 [SMILES: Cc1nc(N2CC3(CCN(c4ncc5cnn(C(F)F)c5n4)C3)C2)cc(C(F)(F)F)n1]

Followed the General procedure A to afford the desired product as a white solid (7.1 mg, 47.11%). 1H NMR (500 MHz, CDCl3) δ 8.80 (s, 1H), 8.02 (s, 1H), 7.57 (t, J=58.0 Hz, 1H), 6.35 (s, 1H), 4.28-4.04 (m, 4H), 3.97-3.73 (m, 4H), 2.59 (s, 3H), 2.34 (t, J=6.9 Hz, 2H). MS m/z: 441 [M+H]⁺.

### Example 651: 6-[2-(difluoromethyl)-2H-pyrazolo[3,4-d]pyrimidin-6-yl]-2-[2-methyl-6-(trifluoromethyl)pyrimidin-4-yl]-2,6-diazaspiro[3.4]octane

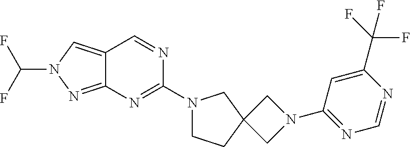 [SMILES: FC(F)n1cc2cnc(N3CCC4(CN(c5cc(C(F)(F)F)ncn5)C4)C3)nc2n1]

Followed the General procedure A to afford the desired product as a white solid (12.3 mg, 47.61%). ¹H NMR (500 MHz, CDCl₃) δ 9.12 (s, 1H), 8.29 (s, 1H), 7.41 (d, J=60.8 Hz, 1H), 6.34 (s, 1H), 4.29-4.06 (m, 4H), 4.01-3.81 (m, 4H), 2.58 (s, 3H), 2.33 (t, J=6.9 Hz, 2H). MS m/z: 441 [M+H]⁺.

### Example 652: 2-[2-methyl-4-(trifluoromethyl)pyrimidin-5-yl]-6-{1-[(3S)-oxolan-3-yl]-1H-pyrazolo[3,4-b]pyrazin-6-yl}-2,6-diazaspiro[3.4]octane

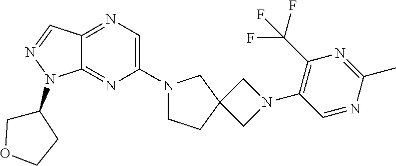 [SMILES: Cc1ncc(N2CC3(CCN(c4cnc5cnn([C@H]6CCOC6)c5n4)C3)C2)c(C(F)(F)F)n1]

Followed the General procedure A to afford the desired product as a off-white solid (12.6 mg, 0.21%). ¹H NMR (500 MHz, CDCl₃) δ 8.10 (s, 1H), 8.03 (s, 1H), 7.95 (s, 1H), 5.42 (ddt, J=9.1, 6.9, 4.7 Hz, 1H), 4.26 (q, J=7.7 Hz, 1H), 4.22-4.08 (m, 6H), 4.03 (td, J=8.2, 5.3 Hz, 1H), 3.89 (d, J=1.3 Hz, 2H), 3.75 (t, J=6.9 Hz, 2H), 2.69 (s, 3H), 2.55 (ddt, J=12.6, 7.6, 4.9 Hz, 1H), 2.50-2.42 (m, 1H), 2.40 (t, J=6.9 Hz, 2H). MS m/z: 461 [M+H]⁺.

### Example 653: 2-[2-methyl-4-(trifluoromethyl)pyrimidin-5-yl]-6-{1-[(3R)-oxolan-3-yl]-1H-pyrazolo[3,4-b]pyrazin-6-yl}-2,6-diazaspiro[3.4]octane

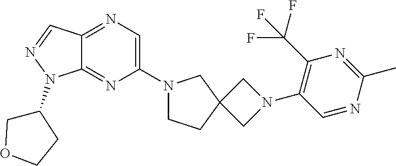 [SMILES: Cc1ncc(N2CC3(CCN(c4cnc5cnn([C@@H]6CCOC6)c5n4)C3)C2)c(C(F)(F)F)n1]

Followed the General procedure A to afford the desired product as a off-white solid (12 mg, 0.2%). ¹H NMR (500 MHz, CDCl₃) δ 8.10 (s, 1H), 8.03 (s, 1H), 7.95 (s, 1H), 5.42 (ddt, J=9.1, 6.9, 4.7 Hz, 1H), 4.30-4.22 (m, 1H), 4.22-4.07 (m, 6H), 4.03 (td, J=8.2, 5.3 Hz, 1H), 3.89 (d, J=1.2 Hz, 2H), 3.74 (t, J=6.9 Hz, 2H), 2.68 (s, 3H), 2.58-2.50 (m, 1H), 2.49-2.42 (m, 1H), 2.40 (t, J=6.9 Hz, 2H). MS m/z: 461 [M+H]⁺.

### Example 654: 6-{1-[(3S)-oxolan-3-yl]-1H-pyrazolo[3,4-b]pyrazin-6-yl}-2-[2-(trifluoromethyl)pyrimidin-5-yl]-2,6-diazaspiro[3.4]octane

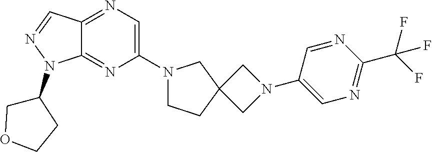 [SMILES: FC(F)(F)c1ncc(N2CC3(CCN(c4cnc5cnn([C@H]6CCOC6)c5n4)C3)C2)cn1]

Followed the General procedure A to afford the desired product as a white solid (21 mg, 0.36%). ¹H NMR (500 MHz, CDCl₃) δ 8.04 (s, 1H), 8.03 (s, 2H), 7.95 (s, 1H), 5.41 (ddt, J=9.1, 6.9, 4.7 Hz, 1H), 4.25 (q, J=7.7 Hz, 1H), 4.21-4.09 (m, 6H), 4.03 (td, J=8.2, 5.3 Hz, 1H), 3.92 (d, J=1.5 Hz, 2H), 3.76 (t, J=6.9 Hz, 2H), 2.55 (ddt, J=12.7, 7.6, 4.9 Hz, 1H), 2.49-2.40 (m, 3H). MS m/z: 447 [M+H]⁺.

### Example 655: 6-{1-[(3R)-oxolan-3-yl]-1H-pyrazolo[3,4-b]pyrazin-6-yl}-2-[2-(trifluoromethyl)pyrimidin-5-yl]-2,6-diazaspiro[3.4]octane

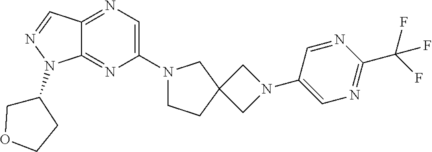 [SMILES: FC(F)(F)c1ncc(N2CC3(CCN(c4cnc5cnn([C@@H]6CCOC6)c5n4)C3)C2)cn1]

Followed the General procedure A to afford the desired product as a off-white solid (20.7 mg, 0.36%). ¹H NMR (500 MHz, CDCl₃) δ 8.04 (s, 1H), 8.02 (s, 2H), 7.95 (s, 1H), 5.41 (ddt, J=9.1, 6.9, 4.7 Hz, 1H), 4.29-4.22 (m, 1H), 4.21-4.08 (m, 6H), 4.03 (td, J=8.2, 5.3 Hz, 1H), 3.94-3.89 (m, 2H), 3.75 (t, J=6.9 Hz, 2H), 2.59-2.50 (m, 1H), 2.50-2.39 (m, 3H). MS m/z: 447 [M+H]⁺.

### Example 656: 2-[4-methyl-2-(trifluoromethyl)pyrimidin-5-yl]-6-{1-[(3S)-oxolan-3-yl]-1H-pyrazolo[3,4-b]pyrazin-6-yl}-2,6-diazaspiro[3.4]octane

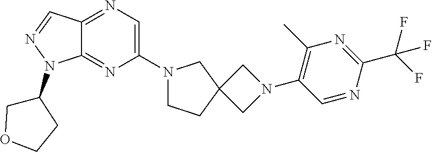 [SMILES: Cc1nc(C(F)(F)F)ncc1N1CC2(CCN(c3cnc4cnn([C@H]5CCOC5)c4n3)C2)C1]

Followed the General procedure A to afford the desired product as an off-white solid (24.2 mg, 0.41%). ¹H NMR (500 MHz, CDCl₃) δ 8.04 (s, 1H), 7.95 (s, 1H), 7.86 (s, 1H), 5.42 (ddt, J=9.0, 6.9, 4.7 Hz, 1H), 4.26 (dt, J=8.6, 7.5 Hz, 1H), 4.22-4.08 (m, 6H), 4.03 (td, J=8.2, 5.3 Hz, 1H), 3.91 (d, J=1.4 Hz, 2H), 3.75 (t, J=6.9 Hz, 2H), 2.54 (s, 4H), 2.49-2.39 (m, 3H). MS m/z: 461 [M+H]⁺.

### Example 657: 2-[4-methyl-2-(trifluoromethyl)pyrimidin-5-yl]-6-{1-[(3R)-oxolan-3-yl]-1H-pyrazolo[3,4-b]pyrazin-6-yl}-2,6-diazaspiro[3.4]octane

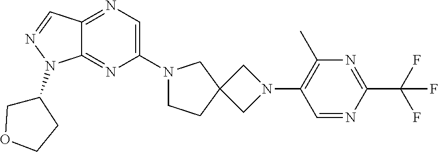 [SMILES: Cc1nc(C(F)(F)F)ncc1N1CC2(CCN(c3cnc4cnn([C@@H]5CCOC5)c4n3)C2)C1]

Followed the General procedure A to afford the desired product as an off-white solid (22.5 mg, 0.38%). ¹H NMR (500 MHz, CDCl₃) δ 8.04 (s, 1H), 7.95 (s, 1H), 7.86 (s, 1H), 5.42 (ddt, J=9.0, 6.9, 4.7 Hz, 1H), 4.26 (dt, J=8.5, 7.4 Hz, 1H), 4.22-4.09 (m, 6H), 4.03 (td, J=8.2, 5.3 Hz, 1H), 3.91 (d, J=1.4 Hz, 2H), 3.75 (t, J=6.9 Hz, 2H), 2.54 (s, 4H), 2.49-2.40 (m, 3H). MS m/z: 461 [M+H]⁺.

### Example 658: 2-[3-methyl-1-(2,2,2-trifluoroethyl)-1H-pyrazolo[3,4-d]pyrimidin-6-yl]-7-[4-methyl-2-(trifluoromethyl)pyrimidin-5-yl]-2,7-diazaspiro[4.4]nonane

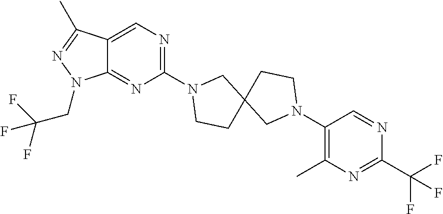 [SMILES: Cc1nc(C(F)(F)F)ncc1N1CCC2(CCN(c3ncc4c(C)nn(CC(F)(F)F)c4n3)C2)C1]

Followed the General procedure A to afford the desired product as a white solid (35.7 mg, 0.55%). ¹H NMR (500 MHz, CDCl₃) δ 8.69 (s, 1H), 8.07 (s, 1H), 4.78 (q, J=8.5 Hz, 2H), 3.92-3.53 (m, 6H), 3.45 (s, 2H), 2.67 (s, 3H), 2.50 (s, 3H), 2.12 (dtt, J=25.9, 12.5, 7.4 Hz, 4H). MS m/z: 501 [M+H]⁺.

### Example 659: 2-[3-methyl-1-(2,2,2-trifluoroethyl)-1H-pyrazolo[3,4-d]pyrimidin-6-yl]-7-[2-methyl-4-(trifluoromethyl)pyrimidin-5-yl]-2,7-diazaspiro[4.4]nonane

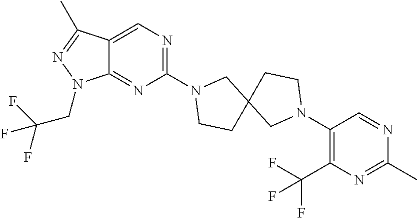 [SMILES: Cc1ncc(N2CCC3(CCN(c4ncc5c(C)nn(CC(F)(F)F)c5n4)C3)C2)c(C(F)(F)F)n1]

Followed the General procedure A to afford the desired product as a white solid (6.5 mg, 0.1%). ¹H NMR (500 MHz, CDCl₃) δ 8.69 (s, 1H), 8.38 (s, 1H), 4.78 (q, J=8.5 Hz, 2H), 3.95-3.52 (m, 6H), 3.44 (s, 2H), 2.67 (s, 3H), 2.50 (s, 3H), 2.19-2.00 (m, 4H). MS m/z: 501 [M+H]⁺.

### Example 660: 6-[1-(oxetan-3-yl)-1H-pyrazolo[3,4-b]pyrazin-6-yl]-2-[6-(trifluoromethyl)pyridin-3-yl]-2,6-diazaspiro[3.4]octane

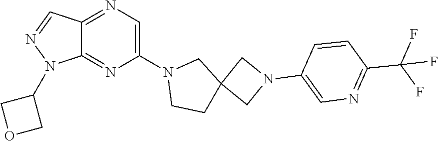 [SMILES: FC(F)(F)c1ccc(N2CC3(CCN(c4cnc5cnn(C6COC6)c5n4)C3)C2)cn1]

Followed the General procedure A to afford the desired product as a white solid (44.3 mg, 0.76%). ¹H NMR (500 MHz, CDCl₃) δ 8.10 (s, 1H), 7.96 (s, 1H), 7.91 (d, J=2.7 Hz, 1H), 7.52-7.47 (m, 1H), 6.81-6.74 (m, 1H), 5.94 (tt, J=8.0, 6.7 Hz, 1H), 5.34 (t, J=6.8 Hz, 2H), 5.10-5.02 (m, 2H), 4.11-4.00 (m, 4H), 3.90 (s, 2H), 3.74 (t, J=6.9 Hz, 2H), 2.41 (t, J=6.9 Hz, 2H). MS m/z: 432 [M+H]⁺.

### Example 661: 8-(1-(2,2-difluoroethyl)-1H-pyrazolo[3,4-b]pyrazin-6-yl)-3,3-dimethyl-2-(4-(trifluoromethyl)pyridin-2-yl)-2,8-diazaspiro[4.5]decan-1-one

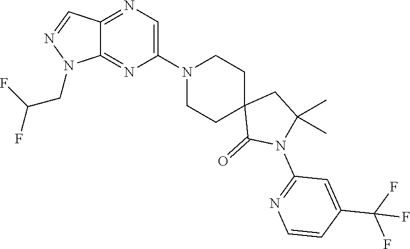 [SMILES: CC1(C)CC2(CCN(c3cnc4cnn(CC(F)F)c4n3)CC2)C(=O)N1c1cc(C(F)(F)F)ccn1]

Step 1: 1-benzyl-4-(2-methylallyl)piperidine-4-carbonitrile: Followed the general procedure AC using 1-benzylpiperidine-4-carbonitrile (2 g, 10 mmol, 1.0 equiv.) and 3-bromo-2-methylprop-1-ene (1.49 g, 11 mmol, 1.1 equiv.) as the starting materials to give 1-benzyl-4-(2-methylallyl)piperidine-4-carbonitrile (1.2 g, 47%) as a colorless oil. MS m/z: 255 [M+H]⁺.

Step 2: 8-benzyl-3,3-dimethyl-2,8-diazaspiro[4.5]decan-1-one: A solution of 1-benzyl-4-(2-methylallyl)piperidine-4-carbonitrile (1.2 g, 4.72 mmol, 1.0 equiv.) in H₂SO₄ (2 mL)/CH₃COOH (4 mL) was stirred for overnight at room temperature. The reaction was quenched with sat. NaHCO₃(aq.) at 0° C. The resulting mixture was extracted with EtOAc (100 mL). The combined organic layers were washed with sat. NaHCO₃(aq.) (2×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 100% gradient in 30 min; detector, UV 254 nm. This resulted in 8-benzyl-3,3-dimethyl-2,8-diazaspiro[4.5]decan-1-one as a colorless oil (800 mg, 62%). MS m/z: 273 [M+H]⁺.

Step 3: 8-benzyl-3,3-dimethyl-2-(4-(trifluoromethyl)pyridin-2-yl)-2,8-diazaspiro[4.5]decan-1-one: Followed the general procedure Y using 8-benzyl-3,3-dimethyl-2,8-diazaspiro[4.5]decan-1-one (200 mg, 0.73 mmol, 1.0 equiv.) and 2-bromo-4-(trifluoromethyl)pyridine (247 mg, 1.09 mmol, 1.5 equiv.) as the starting materials, 1612891-29-8 (14 mg, 18 μmmol, 0.025 equiv.) as the catalyst to give 8-benzyl-3,3-dimethyl-2-(4-(trifluoromethyl)pyridin-2-yl)-2,8-diazaspiro[4.5]decan-1-one (120 mg, 40%) as a white solid MS m/z: 418 [M+H]⁺.

Step 4: 3,3-dimethyl-2-(4-(trifluoromethyl)pyridin-2-yl)-2,8-diazaspiro[4.5]decan-1-one: Followed the general procedure U using 8-benzyl-3,3-dimethyl-2-(4-(trifluoromethyl)pyridin-2-yl)-2,8-diazaspiro[4.5]decan-1-one (70 mg, 0.167 mmol, 1.0 equiv.) as the starting material to give 3,3-dimethyl-2-(4-(trifluoromethyl)pyridin-2-yl)-2,8-diazaspiro[4.5]decan-1-one (40 mg, 73%) as a white solid. MS m/z: 328 [M+H]⁺.

Step 5: 8-(1-(2,2-difluoroethyl)-1H-pyrazolo[3,4-b]pyrazin-6-yl)-3,3-dimethyl-2-(4-(trifluoromethyl)pyridin-2-yl)-2,8-diazaspiro[4.5]decan-1-one: Followed the general procedure I using 3,3-dimethyl-2-(4-(trifluoromethyl)pyridin-2-yl)-2,8-diazaspiro[4.5]decan-1-one (40 mg, 0.121 mmol, 1.0 equiv.) and 6-chloro-1-(2,2-difluoroethyl)-1H-pyrazolo[3,4-b]pyrazine (100 mg, 0.134 mmol, 1.1 equiv.) as the starting materials to give 8-(1-(2,2-difluoroethyl)-1H-pyrazolo[3,4-b]pyrazin-6-yl)-3,3-dimethyl-2-(4-(trifluoromethyl)pyridin-2-yl)-2,8-diazaspiro[4.5]decan-1-one (14.9 mg, 24%) as a white solid. ¹H NMR (400 MHz, DMSO-d₆) δ 8.59-8.48 (m, 2H), 8.15 (s, 1H), 7.35 (dd, J=5.2, 1.6 Hz, 1H), 7.17 (s, 1H), 6.45 (tt, J=54.9, 3.8 Hz, 1H), 4.71 (td, J=15.0, 3.8 Hz, 2H), 4.51 (dt, J=13.7, 4.1 Hz, 2H), 3.38-3.34 (m, 1H), 3.31-3.28 (m, 1H), 2.25 (s, 2H), 2.06-1.97 (m, 2H), 1.95-1.89 (m, 2H), 1.41 (s, 6H). MS m/z: 510.25 [M+H]⁺.

### Example 662: 8-(1-(2,2-difluoroethyl)-1H-pyrazolo[3,4-b]pyrazin-6-yl)-1,1-dimethyl-2-(4-(trifluoromethyl)pyridin-2-yl)-2,8-diazaspiro[4.5]decan-3-one

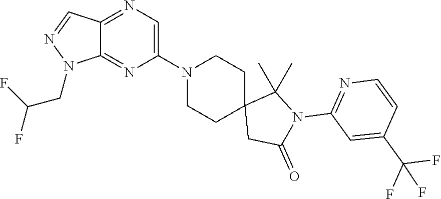 [SMILES: CC1(C)N(c2cc(C(F)(F)F)ccn2)C(=O)CC12CCN(c1cnc3cnn(CC(F)F)c3n1)CC2]

Step 1: 2-(1-benzylpiperidin-4-ylidene)acetonitrile: Followed the general procedure Z using 1-benzylpiperidin-4-one (5 g, 26.4 mmol, 1.0 equiv.) and dimethyl (cyanomethyl)phosphonate (3.94 g, 26.4 mmol, 1.0 equiv.) as the starting materials to give 2-(1-benzylpiperidin-4-ylidene)acetonitrile (5 g, 89%) as a white solid. MS m/z: 213 [M+H]⁺.

Step 2: 2-(1-benzyl-4-(prop-1-en-2-yl)piperidin-4-yl)acetonitrile: To a stirred solution of 2-(1-benzylpiperidin-4-ylidene)acetonitrile (1 g, 4.71 mmol, 1.0 equiv.) and CuI (1.35 g, 7.06 mmol, 1.5 equiv.) in Et₂O (40 mL) were added bromo(prop-1-en-2-yl)magnesium/THF (0.5 M) (23.6 mL, 11.7 mmol, 2.5 equiv.) dropwise at −30° C. under argon atmosphere. The reaction mixture was stirred for 2 h at −30° C. then poured into aq. NH₃(20 ml). The resulting mixture was extracted with EtOAc (50 mL). The combined organic layers were washed with water (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 100% gradient in 40 min; detector, UV 254 nm. This resulted in 2-(1-benzyl-4-(prop-1-en-2-yl)piperidin-4-yl)acetonitrile (0.8 g, 67%) as a colorless oil. MS m/z: 255 [M+H]⁺.

Step 3: 8-benzyl-1,1-dimethyl-2,8-diazaspiro[4.5]decan-3-one: A solution of 2-(1-benzyl-4-(prop-1-en-2-yl)piperidin-4-yl)acetonitrile (800 mg, 3.14 mmol, 1.0 equiv.) in H₂SO₄ (1 mL)/AcOH (2 mL) was stirred for overnight at room temperature. The reaction was quenched by the addition of sat. NaHCO₃(aq.) (50 mL) at 0° C. The resulting mixture was extracted with EtOAc (100 mL). The combined organic layers were washed with sat. NaHCO₃(aq.) (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 100% gradient in 30 min; detector, UV 254 nm. This resulted in 8-benzyl-1,1-dimethyl-2,8-diazaspiro[4.5]decan-3-one (400 mg, 47%) as a colorless oil. MS m/z: 273 [M+H]⁺.

Step 4: 8-benzyl-1,1-dimethyl-2-(4-(trifluoromethyl)pyridin-2-yl)-2,8-diazaspiro[4.5]decan-3-one: Followed the general procedure Y using 8-benzyl-1,1-dimethyl-2,8-diazaspiro[4.5]decan-3-one (200 mg, 0.73 mmol, 1.0 equiv.) and 2-bromo-4-(trifluoromethyl)pyridine (247 mg, 1.1 mmol, 1.5 equiv.) as the starting materials, 1612891-29-8 (30 mg, 0.04 mmol, 0.05 equiv.) as the catalyst to give 8-benzyl-1,1-dimethyl-2-(4-(trifluoromethyl)pyridin-2-yl)-2,8-diazaspiro[4.5]decan-3-one (213 mg, 71%) as a white solid. MS m/z: 418 [M+H]⁺.

Step 5: 1,1-dimethyl-2-(4-(trifluoromethyl)pyridin-2-yl)-2,8-diazaspiro[4.5]decan-3-one: Followed the general procedure U using 8-benzyl-1,1-dimethyl-2-(4-(trifluoromethyl)pyridin-2-yl)-2,8-diazaspiro[4.5]decan-3-one (213 mg, 0.51 mmol, 1.0 equiv.) as the starting material to give 1,1-dimethyl-2-(4-(trifluoromethyl)pyridin-2-yl)-2,8-diazaspiro[4.5]decan-3-one (100 mg, 60%) as a colorless oil. MS m/z: 328 [M+H]⁺.

Step 6: 8-(1-(2,2-difluoroethyl)-1H-pyrazolo[3,4-b]pyrazin-6-yl)-1,1-dimethyl-2-(4-(trifluoromethyl)pyridin-2-yl)-2,8-diazaspiro[4.5]decan-3-one: Followed the general procedure I using 1,1-dimethyl-2-(4-(trifluoromethyl)pyridin-2-yl)-2,8-diazaspiro[4.5]decan-3-one (100 mg, 0.31 mmol, 1.0 equiv.) and 6-chloro-1-(2,2-difluoroethyl)-1H-pyrazolo[3,4-b]pyrazine (73 mg, 414 μmol, 1.1 equiv.) as the starting materials to give 8-(1-(2,2-difluoroethyl)-1H-pyrazolo[3,4-b]pyrazin-6-yl)-1,1-dimethyl-2-(4-(trifluoromethyl)pyridin-2-yl)-2,8-diazaspiro[4.5]decan-3-one (84 mg, 54%) as a white solid. ¹H NMR (400 MHz, DMSO-d₆) δ 8.59 (d, J=5.4 Hz, 1H), 8.47 (d, J=24.6 Hz, 1H), 8.13 (d, J=6.8 Hz, 1H), 7.43-7.33 (m, 1H), 7.21 (d, J=19.3 Hz, 1H), 6.61-6.25 (m, 1H), 4.80-4.45 (m, 4H), 3.18-3.06 (m, 2H), 3.05-2.89 (m, 2H), 1.60 (d, J=18.0 Hz, 4H), 1.27 (d, J=35.1 Hz, 6H). MS m/z: 510.2 [M+H]⁺.

### Example 663: 8-(1-(2,2-difluoroethyl)-1H-pyrazolo[3,4-b]pyrazin-6-yl)-1-ethyl-2-(4-(trifluoromethyl)pyridin-2-yl)-2,8-diazaspiro[4.5]decan-3-one

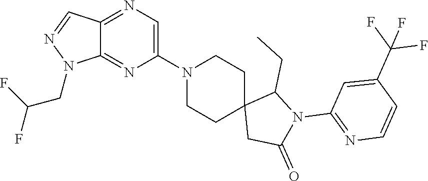 [SMILES: CCC1N(c2cc(C(F)(F)F)ccn2)C(=O)CC12CCN(c1cnc3cnn(CC(F)F)c3n1)CC2]

Step 1: tert-butyl 4-(2-ethoxy-2-oxoethylidene)piperidine-1-carboxylate: Followed the general procedure Z using tert-butyl 4-oxopiperidine-1-carboxylate (3 g, 15 mmol, 1.0 equiv.) and ethyl 2-(diethoxyphosphoryl)acetate (3.37 g, 15 mmol, 1.0 equiv.) as the starting materials to give tert-butyl 4-(2-ethoxy-2-oxoethylidene)piperidine-1-carboxylate (3.2 g, 80%) as a white solid. MS m/z: 270 [M+H]⁺.

Step 2: tert-butyl 4-(2-ethoxy-2-oxoethyl)-4-(1-nitropropyl)piperidine-1-carboxylate: A solution of tert-butyl 4-(2-ethoxy-2-oxoethylidene)piperidine-1-carboxylate (3 g, 11.1 mmol, 1 equiv.), TBAF (55.7 mL, 55.6 mmol, 5 equiv.) and 1-nitropropane (2.48 g, 27.8 mmol, 2.5 equiv) in THF (100 mL) was stirred for overnight at 60° C. The resulting mixture was concentrated under vacuum. The resulting mixture was extracted with EtOAc (200 mL). The combined organic layers were washed with water (3×40 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 100% gradient in 40 min; detector, UV 254 nm. This resulted in tert-butyl 4-(2-ethoxy-2-oxoethyl)-4-(1-nitropropyl)piperidine-1-carboxylate (2 g, 50%) as a colorless oil. MS m/z: 359 [M+H]⁺.

Step 3: tert-butyl 1-ethyl-3-oxo-2,8-diazaspiro[4.5]decane-8-carboxylate: Followed the general procedure AB using tert-butyl 4-(2-ethoxy-2-oxoethyl)-4-(1-nitropropyl)piperidine-1-carboxylate (2 g, 5.5 mmol, 1.0 equiv.) as the starting material to give tert-butyl 1-ethyl-3-oxo-2,8-diazaspiro[4.5]decane-8-carboxylate (1.3 g, 86%) as a colorless oil. MS m/z: 283 [M+H]⁺.

Step 4: tert-butyl 1-ethyl-3-oxo-2-(4-(trifluoromethyl)pyridin-2-yl)-2,8-diazaspiro[4.5]decane-8-carboxylate: Followed the general procedure Y using tert-butyl 1-ethyl-3-oxo-2,8-diazaspiro[4.5]decane-8-carboxylate (1.3 g, 4.7 mmol, 1.0 equiv.) and 2-bromo-4-(trifluoromethyl)pyridine (1.58 g, 7.0 mmol, 1.5 equiv.) as the starting materials as the catalyst to give tert-butyl 1-ethyl-3-oxo-2-(4-(trifluoromethyl)pyridin-2-yl)-2,8-diazaspiro[4.5]decane-8-carboxylate (1.5 g, 75%) as a white solid. MS m/z: 428 [M+H]⁺.

Step 5: 1-ethyl-2-(4-(trifluoromethyl)pyridin-2-yl)-2,8-diazaspiro[4.5]decan-3-one hydrochloride: Followed the general procedure B using tert-butyl 1-ethyl-3-oxo-2-(4-(trifluoromethyl)pyridin-2-yl)-2,8-diazaspiro[4.5]decane-8-carboxylate (1.2 g) as the starting material to give the crude product 1-ethyl-2-(4-(trifluoromethyl)pyridin-2-yl)-2,8-diazaspiro[4.5]decan-3-one hydrochloride (1.2 g). MS m/z: 328 [M+H]⁺.

Step 6: 8-(1-(2,2-difluoroethyl)-1H-pyrazolo[3,4-b]pyrazin-6-yl)-1-ethyl-2-(4-(trifluoromethyl)pyridin-2-yl)-2,8-diazaspiro[4.5]decan-3-one: Followed the general procedure I using 1-ethyl-2-(4-(trifluoromethyl)pyridin-2-yl)-2,8-diazaspiro[4.5]decan-3-one hydrochloride (100 mg, 0.3 mmol, 1.0 equiv.) and 6-chloro-1-(2,2-difluoroethyl)-1H-pyrazolo[3,4-b]pyrazine (72 mg, 0.33 mmol, 1.1 equiv.) as the starting materials to give 8-(1-(2,2-difluoroethyl)-1H-pyrazolo[3,4-b]pyrazin-6-yl)-1-ethyl-2-(4-(trifluoromethyl)pyridin-2-yl)-2,8-diazaspiro[4.5]decan-3-one (70 mg, 45%) as a white solid. ¹H NMR (400 MHz, DMSO-d₆) δ 8.69-8.61 (m, 2H), 8.49 (s, 1H), 8.14 (s, 1H), 7.52 (dd, J=5.1, 1.6 Hz, 1H), 6.44 (tt, J=54.9, 3.8 Hz, 1H), 4.69 (td, J=15.0, 3.8 Hz, 2H), 4.59 (t, J=5.4 Hz, 1H), 4.30-4.10 (m, 2H), 3.60-3.45 (m, 1H), 3.44-3.35 (m, 1H), 2.83 (d, J=17.5 Hz, 1H), 2.71 (d, J=17.5 Hz, 1H), 2.01-1.91 (m, 1H), 1.86-1.77 (m, 2H), 1.72-1.60 (m, 3H), 0.78 (t, J=7.5 Hz, 3H). MS m/z: 509.85 [M+H]⁺.

### Example 664: 8-(1-(2,2-difluoroethyl)-1H-pyrazolo[3,4-b]pyrazin-6-yl)-2-(5-methyl-4-(trifluoromethyl)pyrimidin-2-yl)-2,8-diazaspiro[4.5]decane

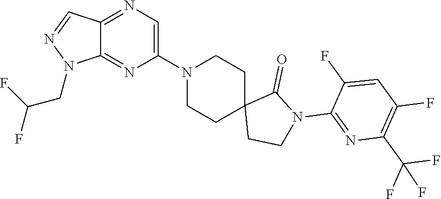 [SMILES: O=C1N(c2nc(C(F)(F)F)c(F)cc2F)CCC12CCN(c1cnc3cnn(CC(F)F)c3n1)CC2]

Step 1: tert-butyl 2-(6-bromo-3,5-difluoropyridin-2-yl)-1-oxo-2,8-diazaspiro[4.5]decane-8-carboxylate: A mixture of 2-bromo-3,5,6-trifluoropyridine (500 mg, 2.36 mmol, 1.0 equiv.), tert-butyl 1-oxo-2,8-diazaspiro[4.5]decane-8-carboxylate (600 mg, 2.36 mmol, 1.0 equiv.) and Cs₂CO₃ (2306 mg, 7.08 mmol, 3.0 equiv.) in DMF (5 mL) was stirred for 3 h at 60° C. under air atmosphere. Desired product could be detected by LCMS. The resulting mixture was extracted with EtOAc (3×20 mL). The combined organic layers were washed with water (2×10 mL), dried over anhydrous Na2SO4. After filtration, the filtrate was concentrated under reduced pressure. The residue was purified by silica gel column chromatography, eluted with EtOAc/PE=1/1 to afford tert-butyl 2-(6-bromo-3,5-difluoropyridin-2-yl)-1-oxo-2,8-diazaspiro[4.5]decane-8-carboxylate (430 mg, 40.9%) as a light yellow oil. MS m/z: 446 [M+H]⁺.

Step 2: tert-butyl 2-(3,5-difluoro-6-(trifluoromethyl)pyridin-2-yl)-1-oxo-2,8-diazaspiro[4.5]decane-8-carboxylate: A mixture of tert-butyl 2-(6-bromo-3,5-difluoropyridin-2-yl)-1-oxo-2,8-diazaspiro[4.5]decane-8-carboxylate (430 mg, 0.963 mmol, 1.0 equiv.), trifluoroiodomethane (944 mg, 4.82 mmol, 5.0 equiv.) and Cu (612 mg, 9.63 mmol, 10.0 equiv.) in DMF (5 mL) was stirred for overnight at 120° C. under argon atmosphere. The resulting mixture was extracted with EtOAc (3×20 mL). The combined organic layers were washed with water (2×10 mL), dried over anhydrous Na₂SO₄. After filtration, the filtrate was concentrated under reduced pressure. The residue was purified by silica gel column chromatography, eluted with EtOAc/PE=1/1 to afford tert-butyl 2-(3,5-difluoro-6-(trifluoromethyl)pyridin-2-yl)-1-oxo-2,8-diazaspiro[4.5]decane-8-carboxylate (65.0 mg, 15.5%) as a light yellow oil. MS m/z: 436 [M+H]⁺.

Step 3: 2-(3,5-difluoro-6-(trifluoromethyl)pyridin-2-yl)-2,8-diazaspiro[4.5]decan-1-one hydrochloride: Followed the general procedure B using tert-butyl 2-(3,5-difluoro-6-(trifluoromethyl)pyridin-2-yl)-1-oxo-2,8-diazaspiro[4.5]decane-8-carboxylate (65.0 mg, 0.149 mmol, 1.0 equiv.) as the starting material to give the crude product 2-(3,5-difluoro-6-(trifluoromethyl)pyridin-2-yl)-2,8-diazaspiro[4.5]decan-1-one hydrochloride (45.0 mg). MS m/z: 336 [M+H]⁺.

Step 4: 2-(3,5-difluoro-6-(trifluoromethyl)pyridin-2-yl)-8-(1-(2,2-difluoroethyl)-1H-pyrazolo[3,4-b]pyrazin-6-yl)-2,8-diazaspiro[4.5]decan-1-one: Followed the general procedure I using 2-(3,5-difluoro-6-(trifluoromethyl)pyridin-2-yl)-2,8-diazaspiro[4.5]decan-1-one hydrochloride (45.0 mg, 0.134 mmol, 1.0 equiv.) and 6-chloro-1-(2,2-difluoroethyl)-1H-pyrazolo[3,4-b]pyrazine (29.3 mg, 0.134 mmol, 1.0 equiv.) as the starting materials to give 2-(3,5-difluoro-6-(trifluoromethyl)pyridin-2-yl)-8-(1-(2,2-difluoroethyl)-1H-pyrazolo[3,4-b]pyrazin-6-yl)-2,8-diazaspiro[4.5]decan-1-one (6.30 mg, 9.01%) as a light yellow oil. ¹H NMR (400 MHz, Chloroform-d) δ 8.24 (d, 1H), 8.06 (s, 1H), 7.49 (t, 1H), 6.22 (tt, 1H), 4.66 (td, 2H), 4.36-4.25 (m, 2H), 4.02 (t, 2H), 3.65-3.53 (m, 2H), 2.28 (t, 2H), 2.18-2.07 (m, 2H), 1.80-1.72 (m, 2H). MS m/z: 518.05 [M+H]⁺.

### Example 665: 8-(1-(2,2-difluoroethyl)-1H-pyrazolo[3,4-b]pyrazin-6-yl)-2-(4-methoxy-6-(trifluoromethyl)pyridin-2-yl)-2,8-diazaspiro[4.5]decan-1-one; Example 666: 8-(1-(2,2-difluoroethyl)-1H-pyrazolo[3,4-b]pyrazin-6-yl)-2-(1-methyl-4-oxo-6-(trifluoromethyl)-1,4-dihydropyridin-2-yl)-2,8-diazaspiro[4.5]decan-1-one

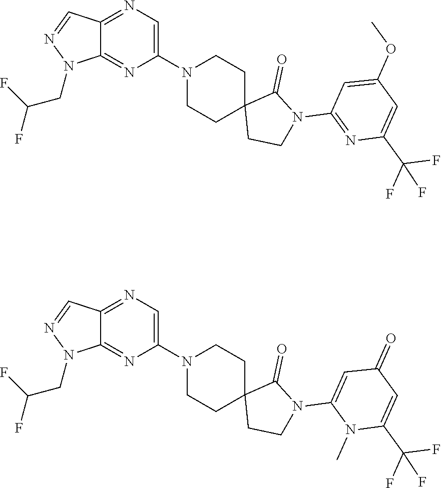 [SMILES: COc1cc(N2CCC3(CCN(c4cnc5cnn(CC(F)F)c5n4)CC3)C2=O)nc(C(F)(F)F)c1 ; Cn1c(N2CCC3(CCN(c4cnc5cnn(CC(F)F)c5n4)CC3)C2=O)cc(=O)cc1C(F)(F)F]

Step 1: tert-butyl 2-(4-hydroxy-6-(trifluoromethyl)pyridin-2-yl)-1-oxo-2,8-diazaspiro[4.5]decane-8-carboxylate: Followed the general procedure I using 2-chloro-6-(trifluoromethyl)pyridin-4-ol (100 mg, 0.506 mmol, 1.0 equiv.) and tert-butyl 1-oxo-2,8-diazaspiro[4.5]decane-8-carboxylate (154 mg, 0.607 mmol, 1.2 equiv.) as the starting materials to give 2-[4-hydroxy-6-(trifluoromethyl)pyridin-2-yl]-1-oxo-2,8-diazaspiro[4.5]decane-8-carboxylate (180 mg, 85.59%) as a light yellow solid. MS m/z: 416 [M+H]⁺.

Step 2: tert-butyl 2-(4-methoxy-6-(trifluoromethyl)pyridin-2-yl)-1-oxo-2,8-diazaspiro[4.5]decane-8-carboxylate; tert-butyl 2-(1-methyl-4-oxo-6-(trifluoromethyl)-1,4-dihydropyridin-2-yl)-1-oxo-2,8-diazaspiro[4.5]decane-8-carboxylate: Followed the general procedure K using CH₃I (111 mg, 0.783 mmol, 1.0 equiv.) and tert-butyl 2-(4-hydroxy-6-(trifluoromethyl)pyridin-2-yl)-1-oxo-2,8-diazaspiro[4.5]decane-8-carboxylate (250 mg, 0.602 mmol, 1.0 equiv.) as the starting materials to give the mixture of tert-butyl 2-[4-methoxy-6-(trifluoromethyl)pyridin-2-yl]-1-oxo-2,8-diazaspiro[4.5]decane-8-carboxylate and tert-butyl 2-(1-methyl-4-oxo-6-(trifluoromethyl)-1,4-dihydropyridin-2-yl)-1-oxo-2,8-diazaspiro[4.5]decane-8-carboxylate (200 mg, 77%) as a light yellow solid. MS m/z: 430 [M+H]⁺.

Step 3: 2-[4-methoxy-6-(trifluoromethyl)pyridin-2-yl]-2,8-diazaspiro[4.5]decan-1-one hydrochloride; 2-(1-methyl-4-oxo-6-(trifluoromethyl)-1,4-dihydropyridin-2-yl)-2,8-diazaspiro[4.5]decan-1-one hydrochloride: Followed the general procedure B using the mixture of tert-butyl 2-[4-methoxy-6-(trifluoromethyl)pyridin-2-yl]-1-oxo-2,8-diazaspiro[4.5]decane-8-carboxylate and tert-butyl 2-(1-methyl-4-oxo-6-(trifluoromethyl)-1,4-dihydropyridin-2-yl)-1-oxo-2,8-diazaspiro[4.5]decane-8-carboxylate (180 mg) as the starting material to give the crude product 2-[4-methoxy-6-(trifluoromethyl)pyridin-2-yl]-2,8-diazaspiro[4.5]decan-1-one hydrochloride and 2-(1-methyl-4-oxo-6-(trifluoromethyl)-1,4-dihydropyridin-2-yl)-2,8-diazaspiro[4.5]decan-1-one hydrochloride (150 mg). MS m/z: 330 [M+H]⁺.

Step 4: 8-(1-(2,2-difluoroethyl)-1H-pyrazolo[3,4-b]pyrazin-6-yl)-2-(4-methoxy-6-(trifluoromethyl)pyridin-2-yl)-2,8-diazaspiro[4.5]decan-1-one; 8-(1-(2,2-difluoroethyl)-1H-pyrazolo[3,4-b]pyrazin-6-yl)-2-(1-methyl-4-oxo-6-(trifluoromethyl)-1,4-dihydropyridin-2-yl)-2,8-diazaspiro[4.5]decan-1-one: Followed the general procedure I using the mixture of 2-[4-methoxy-6-(trifluoromethyl)pyridin-2-yl]-2,8-diazaspiro[4.5]decan-1-one hydrochloride and 2-(1-methyl-4-oxo-6-(trifluoromethyl)-1,4-dihydropyridin-2-yl)-2,8-diazaspiro[4.5]decan-1-one hydrochloride (70 mg, 0.191 mmol, 1.0 equiv.) and 6-chloro-1-(2,2-difluoroethyl)pyrazolo[3,4-b]pyrazine (41.8 mg, 0.191 mmol, 1 equiv.) as the starting materials to give the crude mixture. The crude mixture was further purified by prep-HPLC to give 8-[1-(2,2-difluoroethyl)pyrazolo[3,4-b]pyrazin-6-yl]-2-[4-methoxy-6-(trifluoromethyl)pyridin-2-yl]-2,8-diazaspiro[4.5]decan-1-one (24 mg, 24%) and 8-[1-(2,2-difluoroethyl)pyrazolo[3,4-b]pyrazin-6-yl]-2-[1-methyl-4-oxo-6-(trifluoromethyl)pyridin-2-yl]-2,8-diazaspiro[4.5]decan-1-one (10 mg, 10%) as white solids.

8-(1-(2,2-difluoroethyl)-1H-pyrazolo[3,4-b]pyrazin-6-yl)-2-(4-methoxy-6-(trifluoromethyl)pyridin-2-yl)-2,8-diazaspiro[4.5]decan-1-one: ¹H NMR (400 MHz, Methanol-d₄) δ 8.36 (d, J=2.4 Hz, 1H), 8.03-7.95 (m, 2H), 7.13 (s, 1H), 6.26 (tt, J=55.2 Hz, 4.0 Hz, 1H), 4.75-4.57 (m, 2H), 4.45-4.42 (m, 2H), 4.01-3.86 (m, 5H), 3.46 (t, J=10.2 Hz, 2H), 2.28 (t, J=6.4, 2H), 2.09-1.92 (m, 2H), 1.76-1.73 (m, 2H). MS m/z: 512.2 [M+H]⁺.

8-(1-(2,2-difluoroethyl)-1H-pyrazolo[3,4-b]pyrazin-6-yl)-2-(1-methyl-4-oxo-6-(trifluoromethyl)-1,4-dihydropyridin-2-yl)-2,8-diazaspiro[4.5]decan-1-one: ¹H NMR (400 MHz, Methanol-d₄) δ 8.38 (s, 1H), 8.01-8.00 (m, 2H), 6.58 (s, 1H), 6.26 (tt, J=55.2 Hz, 4.0 Hz, 1H), 4.72-4.64 (m, 2H), 4.46-4.41 (m, 2H), 3.86 (t, J=4.2, 2H), 3.60 (s, 3H), 3.51-3.45 (t, J=6.8, 2H), 2.28 (t, J=7.0, 2H), 2.03-1.95 (m, 2H), 1.78-1.73 (m, 2H). MS m/z: 512.2 [M+H]⁺.

### Example 667: 7-(1-(2,2-difluoroethyl)-1H-pyrazolo[3,4-b]pyrazin-6-yl)-2-(6-(trifluoromethyl)pyridin-2-yl)-2,7-diazaspiro[3.5]nonan-6-one

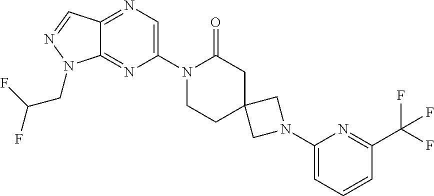 [SMILES: O=C1CC2(CCN1c1cnc3cnn(CC(F)F)c3n1)CN(c1cccc(C(F)(F)F)n1)C2]

Step 1: 2-benzhydryl-7-(1-(2,2-difluoroethyl)-1H-pyrazolo[3,4-b]pyrazin-6-yl)-2,7-diazaspiro[3.5]nonan-6-one: Followed the general procedure I using 2-benzhydryl-2,7-diazaspiro[3.5]nonan-6-one (100 mg, 0.326 mmol, 1.0 equiv.) and 6-chloro-1-(2,2-difluoroethyl)-1H-pyrazolo[3,4-b]pyrazine (78.5 mg, 0.359 mmol, 1.1 equiv.) as the starting materials to give 2-benzhydryl-7-(1-(2,2-difluoroethyl)-1H-pyrazolo[3,4-b]pyrazin-6-yl)-2,7-diazaspiro[3.5]nonan-6-one (80 mg, 50%) as a white solid. MS m/z: 489 [M+H]⁺.

Step 2: 7-(1-(2,2-difluoroethyl)-1H-pyrazolo[3,4-b]pyrazin-6-yl)-2,7-diazaspiro[3.5]nonan-6-one: Followed the general procedure U using 2-benzhydryl-7-(1-(2,2-difluoroethyl)-1H-pyrazolo[3,4-b]pyrazin-6-yl)-2,7-diazaspiro[3.5]nonan-6-one (80 mg, 0.164 mmol, 1.0 equiv.) as the starting material to give 7-(1-(2,2-difluoroethyl)-1H-pyrazolo[3,4-b]pyrazin-6-yl)-2,7-diazaspiro[3.5]nonan-6-one (40 mg, 75%) as a white solid. MS m/z: 323 [M+H]⁺.

Step 3: 7-(1-(2,2-difluoroethyl)-1H-pyrazolo[3,4-b]pyrazin-6-yl)-2-(6-(trifluoromethyl)pyridin-2-yl)-2,7-diazaspiro[3.5]nonan-6-one: Followed the general procedure I using 7-(1-(2,2-difluoroethyl)-1H-pyrazolo[3,4-b]pyrazin-6-yl)-2,7-diazaspiro[3.5]nonan-6-one (40 mg, 0.124 mmol, 1.0 equiv.) and 2-fluoro-6-(trifluoromethyl)pyridine (24.7 mg, 0.149 mmol, 1.2 equiv.) as the starting materials to give 7-(1-(2,2-difluoroethyl)-1H-pyrazolo[3,4-b]pyrazin-6-yl)-2-(6-(trifluoromethyl)pyridin-2-yl)-2,7-diazaspiro[3.5]nonan-6-one (8.8 mg, 15%) as a white solid. ¹H NMR (400 MHz, DMSO-d₆) δ 9.03 (s, 1H), 8.50 (s, 1H), 7.74 (t, J=7.9 Hz, 1H), 7.05 (d, J=7.3 Hz, 1H), 6.73-6.35 (m, 2H), 4.97-4.84 (m, 2H), 4.06-4.00 (m, 2H), 3.97 (d, J=8.2 Hz, 2H), 3.90 (d, J=8.2 Hz, 2H), 2.97 (s, 2H), 2.27 (dd, J=7.0, 5.2 Hz, 2H). MS m/z: 468.05 [M+H]⁺.

### Example 668: 8-(1-(2,2-difluoroethyl)-1H-pyrazolo[3,4-b]pyrazin-6-yl)-7-methyl-2-(6-(trifluoromethyl)pyridin-2-yl)-2,8-diazaspiro[4.5]decan-1-one

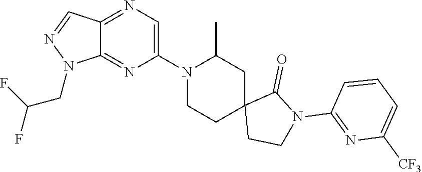 [SMILES: CC1CC2(CCN(c3cccc(C(F)(F)F)n3)C2=O)CCN1c1cnc2cnn(CC(F)F)c2n1]

Step 1: methyl 2-methylpiperidine-4-carboxylate: Followed the general procedure AB using methyl 2-methylisonicotinate (2 g) as the starting materials to give the crude product methyl 2-methylpiperidine-4-carboxylate (2 g) as a colorless oil. MS m/z: 158 [M+H]⁺.

Step 2: 1-(tert-butyl) 4-methyl 2-methylpiperidine-1,4-dicarboxylate: Followed the general procedure R using methyl 2-methylpiperidine-4-carboxylate (1.8 g, 11.4 mmol, 1.0 equiv.) and Boc₂O (2.75 g, 12.6 mmol, 1.1 equiv.) as the starting materials to give 1-(tert-butyl) 4-methyl 2-methylpiperidine-1,4-dicarboxylate (1.5 g, 52%) as a colorless oil. MS m/z: 258 [M+H]⁺.

Step 3: 1-(tert-butyl) 4-methyl 4-(cyanomethyl)-2-methylpiperidine-1,4-dicarboxylate: Followed the general procedure AC using 1-(tert-butyl) 4-methyl 2-methylpiperidine-1,4-dicarboxylate (1 g, 3.89 mmol, 1.0 equiv.) and 2-bromoacetonitrile (926 mg, 7.78 mmol, 2.0 equiv.) as the starting materials to give 1-(tert-butyl) 4-methyl 4-(cyanomethyl)-2-methylpiperidine-1,4-dicarboxylate (500 mg, 43%) as a colorless oil. MS m/z: 297 [M+H]⁺.

Step 4: tert-butyl 7-methyl-1-oxo-2,8-diazaspiro[4.5]decane-8-carboxylate: Followed the general procedure AB using 1-(tert-butyl) 4-methyl 4-(cyanomethyl)-2-methylpiperidine-1,4-dicarboxylate (250 mg) as the starting material to give the crude product tert-butyl 7-methyl-1-oxo-2,8-diazaspiro[4.5]decane-8-carboxylate (150 mg) as a colorless oil. MS m/z: 269 [M+H]⁺.

Step 5: tert-butyl 7-methyl-1-oxo-2-(6-(trifluoromethyl)pyridin-2-yl)-2,8-diazaspiro[4.5]decane-8-carboxylate: Followed the general procedure E using tert-butyl 7-methyl-1-oxo-2,8-diazaspiro[4.5]decane-8-carboxylate (330 g, 1.23 mmol, 1.0 equiv.) and 2-fluoro-6-(trifluoromethyl)pyridine (223 mg, 1.35 mmol, 1.1 equiv.) as the starting materials to give tert-butyl 7-methyl-1-oxo-2-(6-(trifluoromethyl)pyridin-2-yl)-2,8-diazaspiro[4.5]decane-8-carboxylate (130 mg, 25%) as a colorless oil. MS m/z: 414 [M+H]⁺.

Step 6: 7-methyl-2-(6-(trifluoromethyl)pyridin-2-yl)-2,8-diazaspiro[4.5]decan-1-one hydrochloride: Followed the general procedure B using tert-butyl 7-methyl-1-oxo-2-(6-(trifluoromethyl)pyridin-2-yl)-2,8-diazaspiro[4.5]decane-8-carboxylate (120 mg) as the starting material to give the crude product 7-methyl-2-(6-(trifluoromethyl)pyridin-2-yl)-2,8-diazaspiro[4.5]decan-1-one hydrochloride (100 mg). MS m/z: 314 [M+H]⁺.

Step 7: 8-(1-(2,2-difluoroethyl)-1H-pyrazolo[3,4-b]pyrazin-6-yl)-7-methyl-2-(6-(trifluoromethyl)pyridin-2-yl)-2,8-diazaspiro[4.5]decan-1-one: Followed the general procedure I using 7-methyl-2-(6-(trifluoromethyl)pyridin-2-yl)-2,8-diazaspiro[4.5]decan-1-one hydrochloride (100 mg, 0.286 mmol, 1.0 equiv.) and 6-chloro-1-(2,2-difluoroethyl)-1H-pyrazolo[3,4-b]pyrazine (68 mg, 0.315 mmol, 1.1 equiv.) as the starting materials to give 8-(1-(2,2-difluoroethyl)-1H-pyrazolo[3,4-b]pyrazin-6-yl)-7-methyl-2-(6-(trifluoromethyl)pyridin-2-yl)-2,8-diazaspiro[4.5]decan-1-one (25 mg, 17%) as a light yellow solid. ¹H NMR (400 MHz, DMSO-d₆) δ 8.72-8.66 (m, 2H), 8.37 (s, 1H), 8.13 (s, 1H), 7.59-7.47 (m, 1H), 6.45 (tt, J=54.9, 3.8 Hz, 1H), 4.70 (td, J=15.1, 3.8 Hz, 2H), 4.60-4.50 (m, 1H), 4.43-4.32 (m, 1H), 4.09-3.89 (m, 2H), 3.63-3.46 (m, 1H), 2.09-2.00 (m, 2H), 2.00-1.92 (m, 2H), 1.92-1.78 (m, 2H), 1.31 (d, J=6.3 Hz, 3H). MS m/z: 496.05 [M+H]⁺.

### Example 669: rac-(5R,6R)-8-(1-(2,2-difluoroethyl)-1H-pyrazolo[3,4-b]pyrazin-6-yl)-6-methyl-2-(6-(trifluoromethyl)pyridin-2-yl)-2,8-diazaspiro[4.5]decan-1-one, assumed; Example 670: rac-(5S,6R)-8-(1-(2,2-difluoroethyl)-1H-pyrazolo[3,4-b]pyrazin-6-yl)-6-methyl-2-(6-(trifluoromethyl)pyridin-2-yl)-2,8-diazaspiro[4.5]decan-1-one, assumed

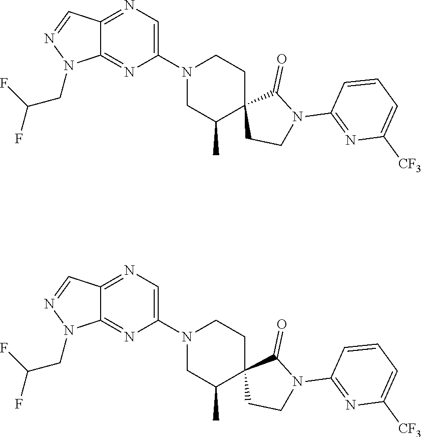 [SMILES: C[C@H]1CN(c2cnc3cnn(CC(F)F)c3n2)CC[C@@]12CCN(c1cccc(C(F)(F)F)n1)C2=O ; C[C@H]1CN(c2cnc3cnn(CC(F)F)c3n2)CC[C@]12CCN(c1cccc(C(F)(F)F)n1)C2=O]

Step 1: methyl 3-methylpiperidine-4-carboxylate: Followed the general procedure AB using methyl 3-methylpyridine-4-carboxylate (2 g, 6.61 mmol, 1.0 equiv.) as the starting material to give methyl 3-methylpiperidine-4-carboxylate (2 g, 95%) as a light yellow oil. MS m/z: 158 [M+H]⁺.

Step 2: 1-tert-butyl 4-methyl 3-methylpiperidine-1,4-dicarboxylate: Followed the general procedure R using methyl 3-methylpiperidine-4-carboxylate (2 g, 12.7 mmol, 1.0 equiv.) and Boc₂O (4.16 g, 19.1 mmol, 1.5 equiv.) as the starting materials to give 1-tert-butyl 4-methyl 3-methylpiperidine-1,4-dicarboxylate (1.6 g, 48%) as a white solid. MS m/z: 258 [M+H]⁺.

Step 3: 1-tert-butyl 4-methyl 4-(cyanomethyl)-3-methylpiperidine-1,4-dicarboxylate: Followed the general procedure AC using 1-tert-butyl 4-methyl 3-methylpiperidine-1,4-dicarboxylate (500 mg, 1.94 mmol, 1.0 equiv.) and 2-bromoacetonitrile (277 mg, 2.33 mmol, 1.2 equiv.) as the starting materials to give 1-tert-butyl 4-methyl 4-(cyanomethyl)-3-methylpiperidine-1,4-dicarboxylate (150 mg, 26%) as a yellow solid. MS m/z: 297 [M+H]⁺.

Step 4: tert-butyl 6-methyl-1-oxo-2,8-diazaspiro[4.5]decane-8-carboxylate: Followed the general procedure AB using 1-tert-butyl 4-methyl 4-(cyanomethyl)-3-methylpiperidine-1,4-dicarboxylate (200 mg, 0.675 mmol, 1.0 equiv.) as the starting material to give tert-butyl 6-methyl-1-oxo-2,8-diazaspiro[4.5]decane-8-carboxylate (130 mg, 71%) as an off-white solid. MS m/z: 269 [M+H]⁺.

Step 5: tert-butyl 6-methyl-1-oxo-2-[6-(trifluoromethyl)pyridin-2-yl]-2,8-diazaspiro[4.5]decane-8-carboxylate: Followed the general procedure E using tert-butyl 6-methyl-1-oxo-2,8-diazaspiro[4.5]decane-8-carboxylate (80 mg, 0.298 mmol, 1.0 equiv.) and 2-fluoro-6-(trifluoromethyl)pyridine (59 mg, 0.358 mmol, 1.2 equiv.) as the starting materials to give tert-butyl 6-methyl-1-oxo-2-[6-(trifluoromethyl)pyridin-2-yl]-2,8-diazaspiro[4.5]decane-8-carboxylate (70 mg, 57%) as an off-white solid. MS m/z: 414 [M+H]⁺.

Step 6: 6-methyl-2-[6-(trifluoromethyl)pyridin-2-yl]-2,8-diazaspiro[4.5]decan-1-one hydrochloride: Followed the general procedure B using tert-butyl 6-methyl-1-oxo-2-[6-(trifluoromethyl)pyridin-2-yl]-2,8-diazaspiro[4.5]decane-8-carboxylate (70 mg) as the starting material to give the crude product 6-methyl-2-[6-(trifluoromethyl)pyridin-2-yl]-2,8-diazaspiro[4.5]decan-1-one hydrochloride (65 mg). MS m/z: 314 [M+H]⁺.

Step 7: rac-(5R,6R)-8-(1-(2,2-difluoroethyl)-1H-pylacolo[3,4-b]pyrazin-6-yl)-6-methyl-2-(6-(trifluoromethyl)pyridin-2-yl)-2,8-diazaspiro[4.5]decan-1-one; rac-(5S,6R)-8-(1-(2,2-difluoroethyl)-1H-pyrazolo[3,4-b]pyrazin-6-yl)-6-methyl-2-(6-(trifluoromethyl)pyridin-2-yl)-2,8-diazaspiro[4.5]decan-1-one: Followed the general procedure I using 6-methyl-2-[6-(trifluoromethyl)pyridin-2-yl]-2,8-diazaspiro[4.5]decan-1-one hydrochloride (65 mg, 0.186 mmol, 1.0 equiv.) and 6-chloro-1-(2,2-difluoroethyl)pyrazolo[3,4-b]pyrazine (40.6 mg, 0.186 mmol, 1 equiv.) as the starting materials to give the crude mixture. The crude mixture was further purified by prep-HPLC to give rac-(5R,6R)-8-(1-(2,2-difluoroethyl)-1H-pyrazolo[3,4-b]pyrazin-6-yl)-6-methyl-2-(6-(trifluoromethyl)pyridin-2-yl)-2,8-diazaspiro[4.5]decan-1-one, assumed (12 mg, 13%) and rac-(5S,6R)-8-(1-(2,2-difluoroethyl)-1H-pyrazolo[3,4-b]pyrazin-6-yl)-6-methyl-2-(6-(trifluoromethyl)pyridin-2-yl)-2,8-diazaspiro[4.5]decan-1-one, assumed (30 mg, 32%) as white solids.

rac-(5R,6R)-8-(1-(2,2-difluoroethyl)-1H-pyrazolo[3,4-b]pyrazin-6-yl)-6-methyl-2-(6-(trifluoromethyl)pyridin-2-yl)-2,8-diazaspiro[4.5]decan-1-one, assumed ¹H NMR (400 MHz, Methanol-d₄) δ 8.62 (d, J=8.8 Hz, 1H), 8.39 (s, 1H), 8.07-7.90 (m, 2H), 7.54 (d, J=7.2, 1H), 6.27 (tt, J=55.2 Hz, 4.0 Hz, 1H), 4.79-4.58 (m, 3H), 4.49-4.45 (m, 1H), 4.11 (t, J=7.2 Hz, 2H), 3.23-3.16 (m, 1H), 2.92-2.86 (m, 1H), 2.40-2.33 (m, 1H), 2.26-2.14 (m, 2H), 2.01-1.83 (m, 2H), 0.96 (d, J=6.8 Hz, 3H). MS m/z: 496.20 [M+H]⁺.

rac-(5S,6R)-8-(1-(2,2-difluoroethyl)-1H-pyrazolo[3,4-b]pyrazin-6-yl)-6-methyl-2-(6-(trifluoromethyl)pyridin-2-yl)-2,8-diazaspiro[4.5]decan-1-one, assumed ¹H NMR (400 MHz, DMSO-d₆) δ 8.64 (d, J=8.8 Hz, 1H), 8.51 (s, 1H), 8.19-8.05 (m, 2H), 7.66 (d, J=7.2, 1H), 6.45 (tt, J=55.2 Hz, 4.0 Hz, 1H), 4.69 (td, J=15.2 Hz, 4.0 Hz, 2H), 4.17-4.13 (m, 2H), 4.02-3.96 (m, 2H), 3.84-3.65 (m, 2H), 2.35-2.15 (m, 2H), 1.96-1.83 (m, 2H), 1.69-1.66 (m, 1H), 1.00 (d, J=6.8 Hz, 3H). MS m/z: 496.20 [M+H]⁺.

### Example 671: 6-(8-(1-(2,2-difluoroethyl)-1H-pyrazolo[3,4-b]pyrazin-6-yl)-1-oxo-2,8-diazaspiro[4.5]decan-2-yl)-N,N-dimethylpicolinamide

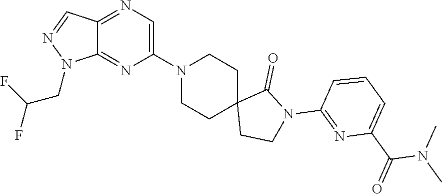 [SMILES: CN(C)C(=O)c1cccc(N2CCC3(CCN(c4cnc5cnn(CC(F)F)c5n4)CC3)C2=O)n1]

Step 1: 6-fluoro-N,N-dimethylpicolinamide: Followed the general procedure G using 6-fluoropicolinic acid (300 mg, 2.12 mmol, 1.0 equiv.) and dimethylamine (107 mg, 2.33 mmol, 1.1 equiv.) as the starting materials to give 6-fluoro-N,N-dimethylpicolinamide (220 mg, 61%) as a white solid. MS m/z: 169 [M+H]⁺.

Step 2: tert-butyl 2-(6-(dimethylcarbamoyl)pyridin-2-yl)-1-oxo-2,8-diazaspiro[4.5]decane-8-carboxylate: Followed the general procedure E using 6-fluoro-N,N-dimethylpicolinamide (220 mg, 1.31 mmol, 1.0 equiv.) and tert-butyl 1-oxo-2,8-diazaspiro[4.5]decane-8-carboxylate (365 mg, 1.43 mmol, 1.1 equiv.) as the starting materials to give tert-butyl 2-(6-(dimethylcarbamoyl)pyridin-2-yl)-1-oxo-2,8-diazaspiro[4.5]decane-8-carboxylate (90 mg, 17%) as a white solid. MS m/z: 403 [M+H]⁺.

Step 3: N,N-dimethyl-6-(1-oxo-2,8-diazaspiro[4.5]decan-2-yl)picolinamide hydrochloride: Followed the general procedure B using tert-butyl 2-(6-(dimethylcarbamoyl)pyridin-2-yl)-1-oxo-2,8-diazaspiro[4.5]decane-8-carboxylate (90 mg) as the starting material to give the crude product N,N-dimethyl-6-(1-oxo-2,8-diazaspiro[4.5]decan-2-yl)picolinamide hydrochloride (60 mg). MS m/z: 303 [M+H]⁺.

Step 4: 6-(8-(1-(2,2-difluoroethyl)-1H-pyrazolo[3,4-b]pyrazin-6-yl)-1-oxo-2,8-diazaspiro[4.5]decan-2-yl)-N,N-dimethylpicolinamide: Followed the general procedure I using N,N-dimethyl-6-(1-oxo-2,8-diazaspiro[4.5]decan-2-yl)picolinamide hydrochloride (60 mg, 0.198 mmol, 1.0 equiv.) and 6-chloro-1-(2,2-difluoroethyl)-1H-pyrazolo[3,4-b]pyrazine (47.7 mg, 0.218 mmol, 1.1 equiv.) as the starting materials to give 6-(8-(1-(2,2-difluoroethyl)-1H-pyrazolo[3,4-b]pyrazin-6-yl)-1-oxo-2,8-diazaspiro[4.5]decan-2-yl)-N,N-dimethylpicolinamide (5.5 mg, 6%) as a white solid. ¹H NMR (400 MHz, DMSO-d₆) δ 8.52 (s, 1H), 8.37 (dd, J=8.4, 0.9 Hz, 1H), 8.15 (s, 1H), 7.97-7.89 (m, 1H), 7.32 (dd, J=7.5, 0.9 Hz, 1H), 6.45 (tt, J=54.9, 3.8 Hz, 1H), 4.77-4.64 (m, 2H), 4.45 (d, J=13.7 Hz, 2H), 4.06-3.98 (m, 2H), 3.33 (d, J=10.7 Hz, 2H), 3.01 (d, J=5.7 Hz, 6H), 2.19 (t, J=7.0 Hz, 2H), 1.90-1.78 (m, 2H), 1.75-1.60 (m, 2H). MS m/z: 485.2 [M+H]⁺.

### Example 672: 8-(1-(2,2-difluoroethyl)-1H-pyrazolo[3,4-b]pyrazin-6-yl)-2-(6-(2-methoxyethoxy)pyridin-2-yl)-2,8-diazaspiro[4.5]decan-1-one

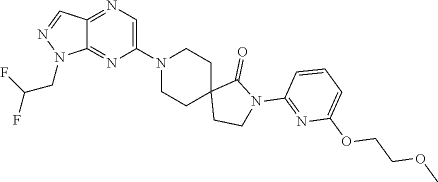 [SMILES: COCCOc1cccc(N2CCC3(CCN(c4cnc5cnn(CC(F)F)c5n4)CC3)C2=O)n1]

Step 1: 2-chloro-6-(2-methoxyethoxy)pyridine: Followed the general procedure C using 6-chloropyridin-2-ol (500 mg, 3.86 mmol, 1.0 equiv.) and 2-methoxyethan-1-ol (442 mg, 5.79 mmol, 1.5 equiv.) as the starting materials to give 2-chloro-6-(2-methoxyethoxy)pyridine (400 mg, 55%) as a yellow oil. MS m/z: 188 [M+H]⁺.

Step 2: tert-butyl 2-(6-(2-methoxyethoxy)pyridin-2-yl)-1-oxo-2,8-diazaspiro[4.5]decane-8-carboxylate: Followed the general procedure Y using 2-chloro-6-(2-methoxyethoxy)pyridine (150 mg, 0.799 mmol, 1.0 equiv.) and tert-butyl 1-oxo-2,8-diazaspiro[4.5]decane-8-carboxylate (224 mg, 0.879 mmol, 1.1 equiv.) as the starting materials, 1612891-29-8 (67.4 mg, 0.08 mmol, 0.1 equiv.) as the catalyst to give tert-butyl 2-(6-(2-methoxyethoxy)pyridin-2-yl)-1-oxo-2,8-diazaspiro[4.5]decane-8-carboxylate as a yellow solid. MS m/z: 406 [M+H]⁺

Step 3: 2-(6-(2-methoxyethoxy)pyridin-2-yl)-2,8-diazaspiro[4.5]decan-1-one hydrochloride: Followed the general procedure B using tert-butyl 2-(6-(2-methoxyethoxy)pyridin-2-yl)-1-oxo-2,8-diazaspiro[4.5]decane-8-carboxylate (150 mg) as the starting material to give the crude product 2-(6-(2-methoxyethoxy)pyridin-2-yl)-2,8-diazaspiro[4.5]decan-1-one hydrochloride (150 mg). MS m/z: 306 [M+H]⁺.

Step 4: 8-(1-(2,2-difluoroethyl)-1H-pyrazolo[3,4-b]pyrazin-6-yl)-2-(6-(2-methoxyethoxy)pyridin-2-yl)-2,8-diazaspiro[4.5]decan-1-one: Followed the general procedure I using 2-(6-(2-methoxyethoxy)pyridin-2-yl)-2,8-diazaspiro[4.5]decan-1-one hydrochloride (50 mg, 0.164 mmol, 1.0 equiv.) and 6-chloro-1-(2,2-difluoroethyl)-1H-pyrazolo[3,4-b]pyrazine (35.8 mg, 0.164 mmol, 1.0 equiv.) as the starting materials to give 8-(1-(2,2-difluoroethyl)-1H-pyrazolo[3,4-b]pyrazin-6-yl)-2-(6-(2-methoxyethoxy)pyridin-2-yl)-2,8-diazaspiro[4.5]decan-1-one (36 mg, 45%) as a white solid. ¹H NMR (400 MHz, DMSO-d₆) δ 8.52 (s, 1H), 8.15 (s, 1H), 7.88 (d, J=7.9 Hz, 1H), 7.71 (t, J=8.0 Hz, 1H), 6.61-6.27 (m, 2H), 4.71 (td, J=15.1, 3.8 Hz, 2H), 4.49-4.35 (m, 4H), 4.02 (t, J=7.0 Hz, 2H), 3.71-3.64 (m, 2H), 3.36 (s, 2H), 3.30 (s, 3H), 2.18 (t, J=7.0 Hz, 2H), 1.88-1.76 (m, 2H), 1.73-1.64 (m, 2H). MS m/z: 488.2 [M+H]⁺.

### Example 673: 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-1-one

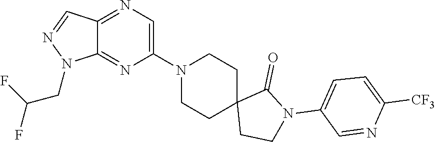 [SMILES: O=C1N(c2ccc(C(F)(F)F)nc2)CCC12CCN(c1cnc3cnn(CC(F)F)c3n1)CC2]

Step 1: tert-butyl 1-oxo-2-(6-(trifluoromethyl)pyridin-3-yl)-2,8-diazaspiro[4.5]decane-8-carboxylate: Followed the general procedure I using tert-butyl 1-oxo-2,8-diazaspiro[4.5]decane-8-carboxylate (100 mg, 0.392 mmol, 1.0 equiv.) and 5-fluoro-2-(trifluoromethyl)pyridine (78.2 mg, 0.471 mmol, 1.2 equiv.) as the starting materials to give tert-butyl 1-oxo-2-(6-(trifluoromethyl)pyridin-3-yl)-2,8-diazaspiro[4.5]decane-8-carboxylate (80 mg, 51%) as a white solid. MS m/z: 400 [M+H]⁺.

Step 2: 2-(6-(trifluoromethyl)pyridin-3-yl)-2,8-diazaspiro[4.5]decan-1-one hydrochloride: Followed the general procedure B using tert-butyl 1-oxo-2-(6-(trifluoromethyl)pyridin-3-yl)-2,8-diazaspiro[4.5]decane-8-carboxylate (80 mg) as the starting material to give the crude product 2-(6-(trifluoromethyl)pyridin-3-yl)-2,8-diazaspiro[4.5]decan-1-one hydrochloride (50 mg). MS m/z: 300 [M+H]⁺.

Step 3: 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-1-one: Followed the general procedure I using 2-(6-(trifluoromethyl)pyridin-3-yl)-2,8-diazaspiro[4.5]decan-1-one hydrochloride (50 mg, 0.167 mmol, 1.0 equiv.) and 6-chloro-1-(2,2-difluoroethyl)-1H-pyrazolo[3,4-b]pyrazine (40.2 mg, 0.184 mmol, 1.1 equiv.) as the starting materials to give 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-1-one (9 mg, 11%) as a white solid. ¹H NMR (400 MHz, DMSO-d₆) δ 9.10 (d, J=2.6 Hz, 1H), 8.50 (d, J=6.0 Hz, 1H), 8.43-8.36 (m, 1H), 8.15 (d, J=3.5 Hz, 1H), 7.95 (dd, J=8.6, 2.8 Hz, 1H), 6.59-6.26 (m, 1H), 4.77-4.63 (m, 2H), 4.44 (d, J=13.5 Hz, 2H), 4.02-3.93 (m, 2H), 3.42-3.31 (m, 2H), 2.28 (t, J=6.9 Hz, 2H), 1.90-1.78 (m, 2H), 1.72 (d, J=13.3 Hz, 2H). MS m/z: 482.1 [M+H]⁺.

### Example 674: 8-(1-(2,2-difluoroethyl)-1H-pyrazolo[3,4-b]pyrazin-6-yl)-2-(5-(trifluoromethyl)pyridin-3-yl)-2,8-diazaspiro[4.5]decan-1-one

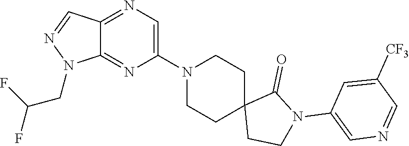 [SMILES: O=C1N(c2cncc(C(F)(F)F)c2)CCC12CCN(c1cnc3cnn(CC(F)F)c3n1)CC2]

Step 1: tert-butyl 1-oxo-2-(5-(trifluoromethyl)pyridin-3-yl)-2,8-diazaspiro[4.5]decane-8-carboxylate: Followed the general procedure F using tert-butyl 1-oxo-2,8-diazaspiro[4.5]decane-8-carboxylate (97.3 mg, 0.590 mmol, 1.5 equiv.) and 3-bromo-5-(trifluoromethyl)pyridine (100 mg, 0.393 mmol, 1.0 equiv.) as the starting materials, 1612891-29-8 (33.0 mg, 0.228 mmol, 0.05 equiv.) as the catalyst to give tert-butyl 1-oxo-2-(5-(trifluoromethyl)pyridin-3-yl)-2,8-diazaspiro[4.5]decane-8-carboxylate (120 mg, 76%) as a white solid. MS m/z: 400 [M+H]⁺.

Step 2: 2-(5-(trifluoromethyl)pyridin-3-yl)-2,8-diazaspiro[4.5]decan-1-one hydrochloride: Followed the general procedure B using tert-butyl 1-oxo-2-(5-(trifluoromethyl)pyridin-3-yl)-2,8-diazaspiro[4.5]decane-8-carboxylate (120 mg) as the starting material to give the crude product 2-(5-(trifluoromethyl)pyridin-3-yl)-2,8-diazaspiro[4.5]decan-1-one hydrochloride (100 mg) as a light brown oil. MS m/z: 300 [M+H]⁺.

Step 3: 8-(1-(2,2-difluoroethyl)-1H-pyrazolo[3,4-b]pyrazin-6-yl)-2-(5-(trifluoromethyl)pyridin-3-yl)-2,8-diazaspiro[4.5]decan-1-one: Followed the general procedure I using 2-(5-(trifluoromethyl)pyridin-3-yl)-2,8-diazaspiro[4.5]decan-1-one hydrochloride (80 mg, 0.238 mmol, 1.0 equiv.) and 6-chloro-1-(2,2-difluoroethyl)-1H-pyrazolo[3,4-b]pyrazine (52 mg, 0.238 mmol, 1.0 equiv.) as the starting materials to give 8-(1-(2,2-difluoroethyl)-1H-pyrazolo[3,4-b]pyrazin-6-yl)-2-(5-(trifluoromethyl)pyridin-3-yl)-2,8-diazaspiro[4.5]decan-1-one (60 mg, 52%) as a white solid. ¹H NMR (400 MHz, DMSO-d₆) δ 9.13 (d, J=2.4 Hz, 1H), 8.75 (d, J=1.9 Hz, 1H), 8.62 (t, J=2.3 Hz, 1H), 8.52 (s, 1H), 8.15 (s, 1H), 6.60-6.16 (m, 1H), 4.78-4.65 (m, 2H), 4.48-4.38 (m, 2H), 4.00 (t, J=6.9 Hz, 2H), 3.41-3.36 (m, 2H), 2.25 (t, J=6.9 Hz, 2H), 1.89-1.78 (m, 2H), 1.76-1.66 (m, 2H). MS m/z: 482.1 [M+H]⁺.

### Example 675: 8-(1-(2,2-difluoroethyl)-1H-pyrazolo[3,4-b]pyrazin-6-yl)-2-(4-(trifluoromethyl)pyridin-3-yl)-2,8-diazaspiro[4.5]decan-1-one

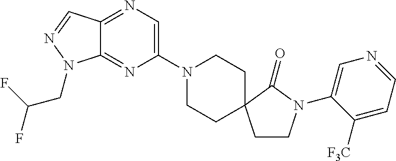 [SMILES: O=C1N(c2cnccc2C(F)(F)F)CCC12CCN(c1cnc3cnn(CC(F)F)c3n1)CC2]

Step 1: tert-butyl 1-oxo-2-(4-(trifluoromethyl)pyridin-3-yl)-2,8-diazaspiro[4.5]decane-8-carboxylate: Followed the general procedure I using tert-butyl 1-oxo-2,8-diazaspiro[4.5]decane-8-carboxylate (120 mg, 0.472 mmol, 1.0 equiv.) and 3-fluoro-4-(trifluoromethyl)pyridine (85 mg, 0.519 mmol, 1.1 equiv.) as the starting materials to give tert-butyl 1-oxo-2-(4-(trifluoromethyl)pyridin-3-yl)-2,8-diazaspiro[4.5]decane-8-carboxylate (60 mg, 32%) as a colorless oil. MS m/z: 400 [M+H]⁺.

Step 2: 2-(4-(trifluoromethyl)pyridin-3-yl)-2,8-diazaspiro[4.5]decan-3-one hydrochloride: Followed the general procedure B using tert-butyl 1-oxo-2-(4-(trifluoromethyl)pyridin-3-yl)-2,8-diazaspiro[4.5]decane-8-carboxylate (60 mg) as the starting material to give the crude product 2-(4-(trifluoromethyl)pyridin-3-yl)-2,8-diazaspiro[4.5]decan-1-one hydrochloride (50 mg). MS m/z: 300 [M+H]⁺.

Step 3: 8-(1-(2,2-difluoroethyl)-1H-pyrazolo[3,4-b]pyrazin-6-yl)-2-(4-(trifluoromethyl)pyridin-3-yl)-2,8-diazaspiro[4.5]decan-1-one: Followed the general procedure I using 2-(4-(trifluoromethyl)pyridin-3-yl)-2,8-diazaspiro[4.5]decan-1-one hydrochloride (50 mg, 0.149 mmol, 1.0 equiv.) and 6-chloro-1-(2,2-difluoroethyl)-1H-pyrazolo[3,4-b]pyrazine (36 mg, 0.164 mol, 1.1 equiv.) as the starting materials to give 8-(1-(2,2-difluoroethyl)-1H-pyrazolo[3,4-b]pyrazin-6-yl)-2-(4-(trifluoromethyl)pyridin-3-yl)-2,8-diazaspiro[4.5]decan-1-one (35 mg, 49%) as a white solid. ¹H NMR (400 MHz, DMSO-d₆) δ 8.92-8.75 (m, 2H), 8.51 (s, 1H), 8.14 (s, 1H), 7.87 (d, J=5.1 Hz, 1H), 6.45 (tt, J=54.9, 3.8 Hz, 1H), 4.71 (td, J=15.1, 3.8 Hz, 2H), 4.50-4.32 (m, 2H), 3.75 (t, J=6.8 Hz, 2H), 2.30 (t, J=6.8 Hz, 2H), 1.88-1.77 (m, 2H), 1.75-1.66 (m, 2H). MS m/z: 482.2[M+H]⁺.

### Example 676: 8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-1-one

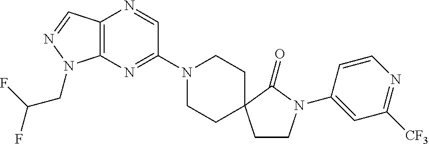 [SMILES: O=C1N(c2ccnc(C(F)(F)F)c2)CCC12CCN(c1cnc3cnn(CC(F)F)c3n1)CC2]

Step 1: tert-butyl 1-oxo-2-(2-(trifluoromethyl)pyridin-4-yl)-2,8-diazaspiro[4.5]decane-8-carboxylate: Followed the general procedure I using tert-butyl 1-oxo-2,8-diazaspiro[4.5]decane-8-carboxylate (100 mg, 0392 mmol, 1.0 equiv.) and 4-fluoro-2-(trifluoromethyl)pyridine (78.1 mg, 0.471 mmol, 1.2 equiv.) as the starting materials to give tert-butyl 1-oxo-2-(2-(trifluoromethyl)pyridin-4-yl)-2,8-diazaspiro[4.5]decane-8-carboxylate (70.0 mg, 44.6%) as a light yellow oil. MS m/z: 400 [M+H]⁺.

Step 2: 2-(2-(trifluoromethyl)pyridin-4-yl)-2,8-diazaspiro[4.5]decan-1-one hydrochloride: Followed the general procedure B using tert-butyl 1-oxo-2-(2-(trifluoromethyl)pyridin-4-yl)-2,8-diazaspiro[4.5]decane-8-carboxylate (70.0 mg, 0.175 mmol, 1.0 equiv.) as the starting material to give the crude product 2-(2-(trifluoromethyl)pyridin-4-yl)-2,8-diazaspiro[4.5]decan-1-one hydrochloride (60.0 mg). MS m/z: 300 [M+H]⁺.

Step 3: 8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-1-one: Followed the general procedure I using 2-(2-(trifluoromethyl)pyridin-4-yl)-2,8-diazaspiro[4.5]decan-1-one hydrochloride (60.0 mg, 0.200 mmol, 1.0 equiv.) and 6-chloro-1-(2,2-difluoroethyl)-1H-pyrazolo[3,4-b]pyrazine (43.8 mg, 0.200 mmol, 1.0 equiv.) as the starting materials to give 8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-1-one (34.2 mg, 39.2%) as a white solid. ¹H NMR (400 MHz, DMSO-d₆) δ 8.93-8.66 (m, 1H), 8.55 (d, J=20.4 Hz, 1H), 8.35 (d, J=2.1 Hz, 1H), 8.15 (s, 1H), 7.97-7.68 (m, 1H), 6.45 (tt, J=54.8, 3.7 Hz, 1H), 4.71 (td, J=15.0, 3.8 Hz, 2H), 4.42 (d, J=13.7 Hz, 2H), 3.96 (t, J=7.0 Hz, 2H), 3.46-3.34 (m, 2H), 2.24 (t, J=7.0 Hz, 2H), 2.02-1.77 (m, 2H), 1.72 (d, J=13.5 Hz, 2H). MS m/z: 482.2 [M+H]⁺.

### Example 677: 8-(1-(2,2-difluoroethyl)-1H-pyrazolo[3,4-b]pyrazin-6-yl)-2-(3-(trifluoromethyl)pyridin-4-yl)-2,8-diazaspiro[4.5]decan-1-one

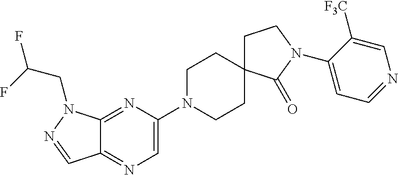 [SMILES: O=C1N(c2ccncc2C(F)(F)F)CCC12CCN(c1cnc3cnn(CC(F)F)c3n1)CC2]

Step 1: tert-butyl 1-oxo-2-(3-(trifluoromethyl)pyridin-4-yl)-2,8-diazaspiro[4.5]decane-8-carboxylate: Followed the general procedure I using tert-butyl 1-oxo-2,8-diazaspiro[4.5]decane-8-carboxylate (100 mg, 0.393 mmol, 1.0 equiv.) and 4-chloro-3-(trifluoromethyl)pyridine (79 mg, 0.432 mmol, 1.1 equiv.) as the starting materials to give tert-butyl 1-oxo-2-(3-(trifluoromethyl)pyridin-4-yl)-2,8-diazaspiro[4.5]decane-8-carboxylate (140 mg, 89%) as a white solid. MS m/z: 400 [M+H]⁺.

Step 2: 2-(3-(trifluoromethyl)pyridin-4-yl)-2,8-diazaspiro[4.5]decan-1-one hydrochloride: Followed the general procedure B using tert-butyl 1-oxo-2-(3-(trifluoromethyl)pyridin-4-yl)-2,8-diazaspiro[4.5]decane-8-carboxylate (140 mg) as the starting material to give the crude product 2-(3-(trifluoromethyl)pyridin-4-yl)-2,8-diazaspiro[4.5]decan-1-one hydrochloride (100 mg). MS m/z: 300 [M+H]⁺.

Step 3: 8-(1-(2,2-difluoroethyl)-1H-pyrazolo[3,4-b]pyrazin-6-yl)-2-(3-(trifluoromethyl)pyridin-4-yl)-2,8-diazaspiro[4.5]decan-1-one: Followed the general procedure I using 2-(3-(trifluoromethyl)pyridin-4-yl)-2,8-diazaspiro[4.5]decan-1-one (100 mg, 0.334 mmol, 1.0 equiv.) and 6-chloro-1-(2,2-difluoroethyl)-1H-pyrazolo[3,4-b]pyrazine (73 mg, 0.334 mmol, 1.0 equiv.) as the starting materials to give 8-(1-(2,2-difluoroethyl)-1H-pyrazolo[3,4-b]pyrazin-6-yl)-2-(3-(trifluoromethyl)pyridin-4-yl)-2,8-diazaspiro[4.5]decan-1-one (38.5 mg, 24%) as a white solid. ¹H NMR (400 MHz, DMSO-d₆) δ 9.03 (s, 1H), 8.97 (d, J=5.3 Hz, 1H), 8.51 (s, 1H), 8.14 (s, 1H), 7.67 (d, J=5.3 Hz, 1H), 6.45 (tt, J=54.9, 3.8 Hz, 1H), 4.83-4.61 (m, 2H), 4.55-4.32 (m, 2H), 3.78 (t, J=6.8 Hz, 2H), 3.51-3.36 (m, 2H), 2.28 (t, J=6.8 Hz, 2H), 1.90-1.73 (m, 2H), 1.69 (d, J=13.4 Hz, 2H). MS m/z: 482.05 [M+H]⁺.

### Example 678: 8-(1-(2,2-difluoroethyl)-1H-pyrazolo[3,4-b]pyrazin-6-yl)-2-(5-(trifluoromethyl)pyridin-2-yl)-2,8-diazaspiro[4.5]decan-1-one

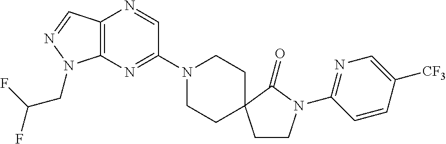 [SMILES: O=C1N(c2ccc(C(F)(F)F)cn2)CCC12CCN(c1cnc3cnn(CC(F)F)c3n1)CC2]

Step 1: tert-butyl 1-oxo-2-(5-(trifluoromethyl)pyridin-2-yl)-2,8-diazaspiro[4.5]decane-8-carboxylate: Followed the general procedure I using tert-butyl 1-oxo-2,8-diazaspiro[4.5]decane-8-carboxylate (120 mg, 0.472 mmol, 1.0 equiv.) and 2-fluoro-5-(trifluoromethyl)pyridine (93.4 mg, 0.566 mmol, 1.2 equiv.) as the starting materials to give tert-butyl 1-oxo-2-(5-(trifluoromethyl)pyridin-2-yl)-2,8-diazaspiro[4.5]decane-8-carboxylate (150 mg, 79%) as a yellow solid. MS m/z: 400 [M+H]⁺.

Step 2: 2-(5-(trifluoromethyl)pyridin-2-yl)-2,8-diazaspiro[4.5]decan-1-one hydrochloride: Followed the general procedure B using tert-butyl 1-oxo-2-(5-(trifluoromethyl)pyridin-2-yl)-2,8-diazaspiro[4.5]decane-8-carboxylate (150 mg) as the starting material to give the crude product 2-(5-(trifluoromethyl)pyridin-2-yl)-2,8-diazaspiro[4.5]decan-1-one hydrochloride (150 mg). MS m/z: 300 [M+H]⁺.

Step 3: 8-(1-(2,2-difluoroethyl)-1H-pyrazolo[3,4-b]pyrazin-6-yl)-2-(5-(trifluoromethyl)pyridin-2-yl)-2,8-diazaspiro[4.5]decan-1-one: Followed the general procedure I using 2-(5-(trifluoromethyl)pyridin-2-yl)-2,8-diazaspiro[4.5]decan-1-one hydrochloride (50 mg, 0.149 mmol, 1.0 equiv.) and 6-chloro-1-(2,2-difluoroethyl)-1H-pyrazolo[3,4-b]pyrazine (32.6 mg, 0.149 mmol, 1.0 equiv.) as the starting materials to give 8-(1-(2,2-difluoroethyl)-1H-pyrazolo[3,4-b]pyrazin-6-yl)-2-(5-(trifluoromethyl)pyridin-2-yl)-2,8-diazaspiro[4.5]decan-1-one (43 mg, 60%) as a white solid. ¹H NMR (400 MHz, DMSO-d₆) δ 8.82 (s, 1H), 8.56-8.49 (m, 2H), 8.22 (dd, J=9.2, 2.5 Hz, 1H), 8.15 (s, 1H), 6.45 (tt, J=55.0, 3.8 Hz, 1H), 4.71 (td, J=15.0, 3.8 Hz, 2H), 4.49-4.39 (m, 2H), 4.07 (t, 2H), 3.41-3.34 (m, 2H), 2.22 (t, J=7.0 Hz, 2H), 1.90-1.78 (m, 2H), 1.78-1.69 (m, 2H). MS m/z: 482.2 [M+H]⁺.

### Example 679: 8-(1-(2,2-difluoroethyl)-1H-pyrazolo[3,4-b]pyrazin-6-yl)-2-(3-(trifluoromethyl)pyridin-2-yl)-2,8-diazaspiro[4.5]decan-1-one

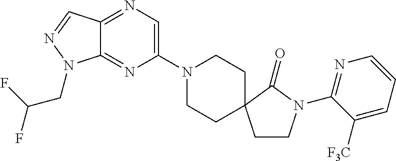 [SMILES: O=C1N(c2ncccc2C(F)(F)F)CCC12CCN(c1cnc3cnn(CC(F)F)c3n1)CC2]

Step 1: tert-butyl 1-oxo-2-(3-(trifluoromethyl)pyridin-2-yl)-2,8-diazaspiro[4.5]decane-8-carboxylate: Followed the general procedure I using 2-fluoro-3-(trifluoromethyl)pyridine (97.3 mg, 0.590 mmol, 1.0 equiv.) and tert-butyl 1-oxo-2,8-diazaspiro[4.5]decane-8-carboxylate (100 mg, 0.393 mmol, 1.0 equiv.) as the starting materials to give tert-butyl 1-oxo-2-[3-(trifluoromethyl)pyridin-2-yl]-2,8-diazaspiro[4.5]decane-8-carboxylate (120 mg, 76%) as a white solid. MS m/z: 400 [M+H]⁺.

Step 2: 2-(3-(trifluoromethyl)pyridin-2-yl)-2,8-diazaspiro[4.5]decan-1-one hydrochloride: Followed the general procedure B using tert-butyl 1-oxo-2-[3-(trifluoromethyl)pyridin-2-yl]-2,8-diazaspiro[4.5]decane-8-carboxylate (120 mg) as the starting material to give the crude product 2-[3-(trifluoromethyl)pyridin-2-yl]-2,8-diazaspiro[4.5]decan-1-one hydrochloride (100 mg). MS m/z: 300 [M+H]⁺.

Step 3: 8-(1-(2,2-difluoroethyl)-1H-pyrazolo[3,4-b]pyrazin-yl)-2-(3-(trifluoromethyl)pyridin-2-yl)-2,8-diazaspiro[4.5]decan-1-one: Followed the general procedure I using 2-[3-(trifluoromethyl)pyridin-2-yl]-2,8-diazaspiro[4.5]decan-1-one hydrochloride (80 mg, 0.238 mmol, 1.0 equiv.) and 6-chloro-1-(2,2-difluoroethyl)pyrazolo[3,4-b]pyrazine (52.1 mg, 0.238 mmol, 1 equiv.) as the starting materials to give 8-(1-(2,2-difluoroethyl)-1H-pyrazolo[3,4-b]pyrazin-6-yl)-2-(3-(trifluoromethyl)pyridin-2-yl)-2,8-diazaspiro[4.5]decan-1-one (60 mg, 52%) as a white solid. ¹H NMR (400 MHz, DMSO-d₆) δ 8.83 (d, 1H), 8.51 (s, 1H), 8.33 (d, 1H), 8.14 (s, 1H), 7.66 (d, 1H), 6.45 (tt, 1H), 4.70 (td, 2H), 4.53-4.34 (m, 2H), 3.86-3.83 (m, 2H), 3.43-3.36 (m, 2H), 2.31-3.27 (m, 2H), 1.88-1.75 (m, 2H), 1.69-1.66 (m, 2H). MS m/z: 482.2 [M+H]⁺.

### Example 680: 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-3-one

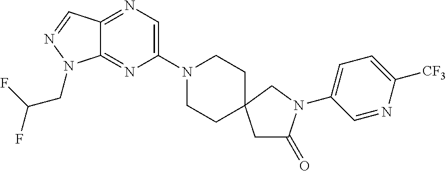 [SMILES: O=C1CC2(CCN(c3cnc4cnn(CC(F)F)c4n3)CC2)CN1c1ccc(C(F)(F)F)nc1]

Step 1: tert-butyl 3-oxo-2-(6-(trifluoromethyl)pyridin-3-yl)-2,8-diazaspiro[4.5]decane-8-carboxylate: Followed the general procedure I using tert-butyl 3-oxo-2,8-diazaspiro[4.5]decane-8-carboxylate (100 mg, 0.392 mmol, 1.0 equiv.) and 5-fluoro-2-(trifluoromethyl)pyridine (78.2 mg, 0.471 mmol, 1.2 equiv.) as the starting materials to give tert-butyl 3-oxo-2-(6-(trifluoromethyl)pyridin-3-yl)-2,8-diazaspiro[4.5]decane-8-carboxylate (80 mg, 51%) as a white solid. MS m/z: 400 [M+H]⁺.

Step 2: 2-(6-(trifluoromethyl)pyridin-3-yl)-2,8-diazaspiro[4.5]decan-3-one hydrochloride: Followed the general procedure B using tert-butyl 3-oxo-2-(6-(trifluoromethyl)pyridin-3-yl)-2,8-diazaspiro[4.5]decane-8-carboxylate (80 mg) as the starting material to give the crude product 2-(6-(trifluoromethyl)pyridin-3-yl)-2,8-diazaspiro[4.5]decan-3-one hydrochloride (50 mg). MS m/z: 300 [M+H]⁺.

Step 3: 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-3-one: Followed the general procedure I using 2-(6-(trifluoromethyl)pyridin-3-yl)-2,8-diazaspiro[4.5]decan-3-one hydrochloride (50 mg, 0.167 mmol, 1.00 equiv.) and 6-chloro-1-(2,2-difluoroethyl)-1H-pyrazolo[3,4-b]pyrazine (40.2 mg, 0.184 mmol, 1.10 equiv.) as the starting materials to give 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-3-one (22 mg, 27%) as a white solid. ¹H NMR (400 MHz, DMSO-d₆) δ 9.05 (d, J=2.5 Hz, 1H), 8.51 (s, 1H), 8.40 (d, J=8.8 Hz, 1H), 8.14 (s, 1H), 7.94 (d, J=8.7 Hz, 1H), 6.60-6.27 (m, 1H), 4.77-4.64 (m, 2H), 3.96-3.86 (m, 4H), 3.81-3.73 (m, 2H), 2.64 (s, 2H), 1.76 (t, J=5.6 Hz, 4H). MS m/z: 482.1 [M+H]⁺.

### Example 681: 8-(1-(2,2-difluoroethyl)-1H-pyrazolo[3,4-b]pyrazin-6-yl)-2-(5-(trifluoromethyl)pyridin-3-yl)-2,8-diazaspiro[4.5]decan-3-one

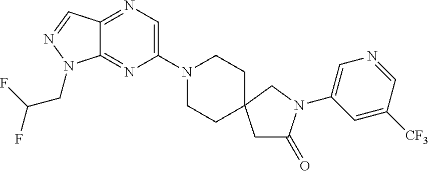 [SMILES: O=C1CC2(CCN(c3cnc4cnn(CC(F)F)c4n3)CC2)CN1c1cncc(C(F)(F)F)c1]

Step 1: tert-butyl 3-oxo-2-(5-(trifluoromethyl)pyridin-3-yl)-2,8-diazaspiro[4.5]decane-8-carboxylate: Followed the general procedure F using tert-butyl 3-oxo-2,8-diazaspiro[4.5]decane-8-carboxylate (97.3 mg, 0.590 mmol, 1.5 equiv.) and 3-bromo-5-(trifluoromethyl)pyridine (100 mg, 0.393 mmol, 1.0 equiv.) as the starting materials, 1612891-29-8 (33 mg, 0.228 mmol, 0.05 equiv.) as the catalyst to give tert-butyl 3-oxo-2-(5-(trifluoromethyl)pyridin-3-yl)-2,8-diazaspiro[4.5]decane-8-carboxylate (120 mg, 76%) as a white m/z: 400[M+H]⁺.

Step 2: 2-(5-(trifluoromethyl)pyridin-3-yl)-2,8-diazaspiro[4.5]decan-3-one hydrochloride: Followed the general procedure B using tert-butyl 3-oxo-2-(5-(trifluoromethyl)pyridin-3-yl)-2,8-diazaspiro[4.5]decane-8-carboxylate (120 mg) as the starting material to give the crude product 2-(5-(trifluoromethyl)pyridin-3-yl)-2,8-diazaspiro[4.5]decan-3-one hydrochloride (100 mg). MS m/z: 300 [M+H]⁺.

Step 3: 8-(1-(2,2-difluoroethyl)-1H-pyrazolo[3,4-b]pyrazin-6-yl)-2-(5-(trifluoromethyl)pyridin-3-yl)-2,8-diazaspiro[4.5]decan-3-one: Followed the general procedure I using 2-(5-(trifluoromethyl)pyridin-3-yl)-2,8-diazaspiro[4.5]decan-3-one hydrochloride (80 mg, 0.238 mmol, 1.0 equiv.) and 6-chloro-1-(2,2-difluoroethyl)-1H-pyrazolo[3,4-b]pyrazine (52 mg, 0.238 mmol, 1.0 equiv.) as the starting materials to give 8-(1-(2,2-difluoroethyl)-1H-pyrazolo[3,4-b]pyrazin-6-yl)-2-(5-(trifluoromethyl)pyridin-3-yl)-2,8-diazaspiro[4.5]decan-3-one (60 mg, 52%) as a white solid. ¹H NMR (400 MHz, DMSO-d₆) δ 9.15 (d, J=2.4 Hz, 1H), 8.74 (d, J=2.0 Hz, 1H), 8.56 (d, J=2.3 Hz, 1H), 8.51 (s, 1H), 8.14 (s, 1H), 6.70-6.23 (m, 1H), 4.77-4.64 (m, 2H), 3.95-3.75 (m, 6H), 2.62 (s, 2H), 1.83-1.69 (m, 4H). MS m/z: 482.1 [M+H]⁺.

### Example 682: 8-(1-(2,2-difluoroethyl)-1H-pyrazolo[3,4-b]pyrazin-6-yl)-2-(4-(trifluoromethyl)pyridin-3-yl)-2,8-diazaspiro[4.5]decan-3-one

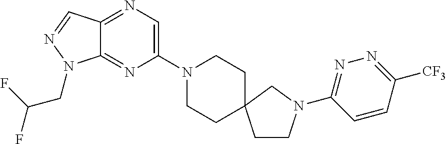 [SMILES: FC(F)Cn1ncc2ncc(N3CCC4(CCN(c5ccc(C(F)(F)F)nn5)C4)CC3)nc21]

Step 1: tert-butyl 3-oxo-2-(4-(trifluoromethyl)pyridin-3-yl)-2,8-diazaspiro[4.5]decane-8-carboxylate: Followed the general procedure I using tert-butyl 3-oxo-2,8-diazaspiro[4.5]decane-8-carboxylate (120 mg, 0.472 mmol, 1.0 equiv.) and 3-fluoro-4-(trifluoromethyl)pyridine (85 mg, 0.519 mmol, 1.1 equiv.) as the starting materials to give tert-butyl 3-oxo-2-(4-(trifluoromethyl)pyridin-3-yl)-2,8-diazaspiro[4.5]decane-8-carboxylate (60 mg, 32%) as a colorless oil. MS m/z: 400 [M+H]⁺.

Step 2: 2-(4-(trifluoromethyl)pyridin-3-yl)-2,8-diazaspiro[4.5]decan-3-one hydrochloride: Followed the general procedure B using tert-butyl 3-oxo-2-(4-(trifluoromethyl)pyridin-3-yl)-2,8-diazaspiro[4.5]decane-8-carboxylate (60 mg) as the starting material to give the crude product 2-(4-(trifluoromethyl)pyridin-3-yl)-2,8-diazaspiro[4.5]decan-3-one hydrochloride (50 mg). MS m/z: 300 [M+H]⁺.

Step 3: 8-(1-(2,2-difluoroethyl)-1H-pyrazolo[3,4-b]pyrazin-6-yl)-2-(4-(trifluoromethyl)pyridin-3-yl)-2,8-diazaspiro[4.5]decan-3-one: Followed the general procedure I using 2-(4-(trifluoromethyl)pyridin-3-yl)-2,8-diazaspiro[4.5]decan-3-one hydrochloride (50 mg, 0.149 mmol, 1.0 equiv.) and 6-chloro-1-(2,2-difluoroethyl)-1H-pyrazolo[3,4-b]pyrazine (36 mg, 0.164 mol, 1.1 equiv.) as the starting materials to give 8-(1-(2,2-difluoroethyl)-1H-pyrazolo[3,4-b]pyrazin-6-yl)-2-(4-(trifluoromethyl)pyridin-3-yl)-2,8-diazaspiro[4.5]decan-3-one (14 mg, 20%) as a white solid. ¹H NMR (400 MHz, DMSO-d₆) δ 8.88-8.84 (m, 2H), 8.51 (s, 1H), 8.14 (s, 1H), 7.88 (d, J=5.2 Hz, 1H), 6.45 (tt, J=54.9, 3.8 Hz, 1H), 4.71 (td, J=15.0, 3.8 Hz, 2H), 4.00-3.88 (m, 2H), 3.82-3.69 (m, 2H), 3.65 (s, 2H), 2.55 (s, 2H), 1.79 (t, J=5.7 Hz, 4H). MS m/z: 482.2[M+H]⁺.

### Example 683: 8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-3-one

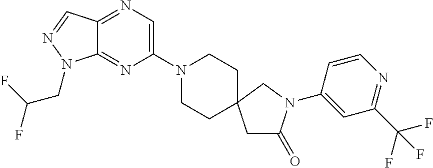 [SMILES: O=C1CC2(CCN(c3cnc4cnn(CC(F)F)c4n3)CC2)CN1c1ccnc(C(F)(F)F)c1]

Step 1: tert-butyl 3-oxo-2-(2-(trifluoromethyl)pyridin-4-yl)-2,8-diazaspiro[4.5]decane-8-carboxylate: Followed the general procedure I using tert-butyl 3-oxo-2,8-diazaspiro[4.5]decane-8-carboxylate (200 mg, 0.786 mmol, 1.0 equiv.) and 4-fluoro-2-(trifluoromethyl)pyridine (195 mg, 1.18 mmol, 1.5 equiv.) as the starting materials to give tert-butyl 3-oxo-2-(2-(trifluoromethyl)pyridin-4-yl)-2,8-diazaspiro[4.5]decane-8-carboxylate (130 mg, 41.4%) as a light yellow oil. MS m/z: 400 [M+H]⁺.

Step 2: 2-(2-(trifluoromethyl)pyridin-4-yl)-2,8′diazaspiro[4.5]decan-3-one hydrochloride: Followed the general procedure B using tert-butyl 3-oxo-2-(2-(trifluoromethyl)pyridin-4-yl)-2,8-diazaspiro[4.5]decane-8-carboxylate (130 mg, 0.325 mmol, 1.0 equiv.) as the starting material to give the crude product 2-(2-(trifluoromethyl)pyridin-4-yl)-2,8-diazaspiro[4.5]decan-3-one hydrochloride (100 mg). MS m/z: 300 [M+H]⁺.

Step 3: 8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-3-one: Followed the general procedure I using 2-(2-(trifluoromethyl)pyridin-4-yl)-2,8-diazaspiro[4.5]decan-3-one hydrochloride (100 mg, 0.298 mmol, 1.0 equiv.) and 6-chloro-1-(2,2-difluoroethyl)-1H-pyrazolo[3,4-b]pyrazine (65.1 mg, 0.298 mmol, 1.0 equiv.) as the starting materials to give 8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-3-one (31.4 mg, 21.5%) as a white solid. ¹H NMR (400 MHz, DMSO-d₆) δ 8.69 (d, J=5.6 Hz, 1H), 8.51 (s, 1H), 8.27 (d, J=2.1 Hz, 1H), 8.14 (s, 1H), 7.89 (dd, J=5.7, 2.2 Hz, 1H), 6.41 (m, 1H), 4.70 (td, J=15.0, 3.8 Hz, 2H), 3.99-3.68 (m, 6H), 2.67 (s, 2H), 1.74 (d, J=6.7 Hz, 4H). MS m/z: 482.2 [M+H]⁺.

### Example 684: 8-(1-(2,2-difluoroethyl)-1H-pyrazolo[3,4-b]pyrazin-6-yl)-2-(3-(trifluoromethyl)pyridin-4-yl)-2,8-diazaspiro[4.5]decan-1-one

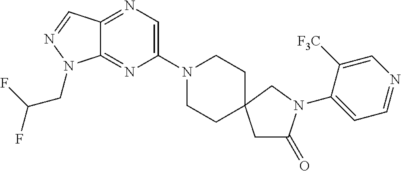 [SMILES: O=C1CC2(CCN(c3cnc4cnn(CC(F)F)c4n3)CC2)CN1c1ccncc1C(F)(F)F]

Step 1: tert-butyl 3-oxo-2-(3-(trifluoromethyl)pyridin-4-yl)-2,8-diazaspiro[4.5]decane-8-carboxylate: Followed the general procedure I using tert-butyl 3-oxo-2,8-diazaspiro[4.5]decane-8-carboxylate (100 mg, 0.393 mmol, 1.0 equiv.) and 4-chloro-3-(trifluoromethyl)pyridine (79 mg, 0.432 mmol, 1.1 equiv.) as the starting materials to give tert-butyl 3-oxo-2-(3-(trifluoromethyl)pyridin-4-yl)-2,8-diazaspiro[4.5]decane-8-carboxylate (130 mg, 83%) as a white solid. MS m/z: 400 [M+H]⁺.

Step 2: 2-(3-(trifluoromethyl)pyridin-4-yl)-2,8-diazaspiro[4.5]decan-3-one hydrochloride: Followed the general procedure B using tert-butyl 3-oxo-2-(3-(trifluoromethyl)pyridin-4-yl)-2,8-diazaspiro[4.5]decane-8-carboxylate (130 mg) as the starting material to give the crude product 2-(3-(trifluoromethyl)pyridin-4-yl)-2,8-diazaspiro[4.5]decan-3-one (80 mg). MS m/z: 300 [M+H]⁺.

Step 3: 8-(1-(2,2-difluoroethyl)-1H-pyrazolo[3,4-b]pyrazin-6-yl)-2-(3-(trifluoromethyl)pyridin-4-yl)-2,8-diazaspiro[4.5]decan-3-one: Followed the general procedure I using 2-(3-(trifluoromethyl)pyridin-4-yl)-2,8-diazaspiro[4.5]decan-3-one hydrochloride (80 mg, 0.267 mmol, 1.0 equiv.) and 6-chloro-1-(2,2-difluoroethyl)-1H-pyrazolo[3,4-b]pyrazine (58.4 mg, 0.267 mmol, 1.0 equiv.) as the starting materials to give 8-(1-(2,2-difluoroethyl)-1H-pyrazolo[3,4-b]pyrazin-6-yl)-2-(3-(trifluoromethyl)pyridin-4-yl)-2,8-diazaspiro[4.5]decan-3-one (18.4 mg, 14%) as a white solid. ¹H NMR (400 MHz, DMSO-d₆) δ 9.02 (s, 1H), 8.96 (d, J=5.3 Hz, 1H), 8.50 (s, 1H), 8.13 (s, 1H), 7.67 (d, J=5.3 Hz, 1H), 6.44 (tt, J=54.9, 3.8 Hz, 1H), 4.77-4.64 (m, 2H), 3.99-3.89 (m, 2H), 3.77-3.71 (m, 2H), 3.69-3.64 (m, 2H), 2.54 (s, 2H), 1.80-1.75 (m, 4H). MS m/z: 482.15 [M+H]⁺.

### Example 685: 8-(1-(2,2-difluoroethyl)-1H-pyrazolo[3,4-b]pyrazin-6-yl)-2-(5-(trifluoromethyl)pyridin-2-yl)-2,8-diazaspiro[4.5]decan-3-one

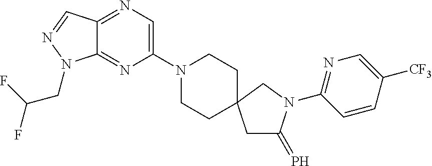

Step 1: tert-butyl 1-oxo-2-(5-(trifluoromethyl)pyridin-2-yl)-2,8-diazaspiro[4.5]decane-8-carboxylate: Followed the general procedure I using tert-butyl 3-oxo-2,8-diazaspiro[4.5]decane-8-carboxylate (120 mg, 0.472 mmol, 1.0 equiv.) and 2-fluoro-5-(trifluoromethyl)pyridine (93.4 mg, 0.566 mmol, 1.2 equiv.) as the starting materials to give tert-butyl 3-oxo-2-(5-(trifluoromethyl)pyridin-2-yl)-2,8-diazaspiro[4.5]decane-8-carboxylate (157 mg, 83%) as a white solid. MS m/z: 400 [M+H]⁺.

Step 2: 2-(5-(trifluoromethyl)pyridin-2-yl)-2,8-diazaspiro[4.5]decan-3-one hydrochloride: Followed the general procedure B using tert-butyl 3-oxo-2-(5-(trifluoromethyl)pyridin-2-yl)-2,8-diazaspiro[4.5]decane-8-carboxylate (157 mg) as the starting material to give the crude product 2-(5-(trifluoromethyl)pyridin-2-yl)-2,8-diazaspiro[4.5]decan-3-one hydrochloride (157 mg). MS m/z: 300 [M+H]⁺.

Step 3: 8-(1-(2,2-difluoroethyl)-1H-pyrazolo[3,4-b]pyrazin-6-yl)-2-(5-(trifluoromethyl)pyridin-2-yl)-2,8-diazaspiro[4.5]decan-3-one: Followed the general procedure I using 2-(5-(trifluoromethyl)pyridin-2-yl)-2,8-diazaspiro[4.5]decan-3-one hydrochloride (50 mg, 0.149 mmol, 1.0 equiv.) and 6-chloro-1-(2,2-difluoroethyl)-1H-pyrazolo[3,4-b]pyrazine (32.6 mg, 0.149 mmol, 1.0 equiv.) as the starting materials to give 8-(1-(2,2-difluoroethyl)-1H-pyrazolo[3,4-b]pyrazin-6-yl)-2-(5-(trifluoromethyl)pyridin-2-yl)-2,8-diazaspiro[4.5]decan-3-one (43 mg, 60%) as a white solid. ¹H NMR (400 MHz, DMSO-d₆) δ 8.80-8.75 (m, 1H), 8.56-8.47 (m, 2H), 8.26-8.19 (m, 1H), 8.13 (s, 1H), 6.44 (tt, J=54.9, 3.8 Hz, 1H), 4.70 (td, J=15.0, 3.8 Hz, 2H), 4.09-3.90 (m, 4H), 3.74-3.64 (m, 2H), 2.72 (s, 2H), 1.82-1.69 (m, 4H). MS m/z: 482.1 [M+H]⁺.

### Example 686: 8-(1-(2,2-difluoroethyl)-1H-pyrazolo[3,4-b]pyrazin-6-yl)-2-(3-(trifluoromethyl)pyridin-2-yl)-2,8-diazaspiro[4.5]decan-3-one

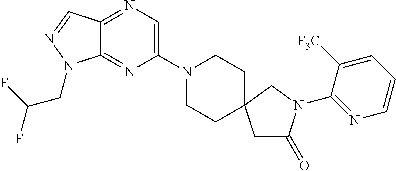 [SMILES: O=C1CC2(CCN(c3cnc4cnn(CC(F)F)c4n3)CC2)CN1c1ncccc1C(F)(F)F]

Step 1: 8-(1-(2,2-difluoroethyl)-1H-pyrazolo[3,4-b]pyrazin-6-yl)-2-(3-(trifluoromethyl)pyridin-2-yl)-2,8-diazaspiro[4.5]decan-3-one: Followed the general procedure I using 8-(1-(2,2-difluoroethyl)-1H-pyrazolo[3,4-b]pyrazin-6-yl)-2,8-diazaspiro[4.5]decan-3-one (80 mg, 0.238 mmol, 1.0 equiv.) and 2-fluoro-3-(trifluoromethyl)pyridine (39 mg, 0.238 mmol, 1.0 equiv.) as the starting materials to give 8-(1-(2,2-difluoroethyl)-1H-pyrazolo[3,4-b]pyrazin-6-yl)-2-(3-(trifluoromethyl)pyridin-2-yl)-2,8-diazaspiro[4.5]decan-3-one (60 mg, 52%) as a white solid. ¹H NMR (400 MHz, DMSO-d₆) δ 8.81 (d, 1H), 8.51 (s, 1H), 8.33 (d, 1H), 8.13 (s, 1H), 7.67 (d, 1H), 6.44 (tt, 1H), 4.70 (td, 2H), 3.98-3.92 (m, 2H), 3.77-3.71 (m, 4H), 2.55 (s, 2H), 1.80-1.78 (m, 4H). MS m/z: 482.1 [M+H]⁺.

### Example 687: 8-(1-(2,2-difluoroethyl)-1H-pyrazolo[3,4-b]pyrazin-6-yl)-2-((3-(trifluoromethyl)pyridin-4-yl)oxy)-8-azaspiro[4.5]decane

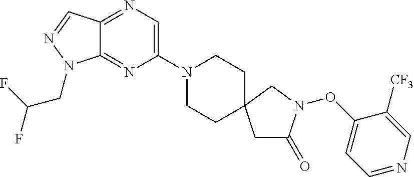 [SMILES: O=C1CC2(CCN(c3cnc4cnn(CC(F)F)c4n3)CC2)CN1Oc1ccncc1C(F)(F)F]

Step 1: tert-butyl 2-((3-(trifluoromethyl)pyridin-4-yl)oxy)-8-azaspiro[4.5]decane-8-carboxylate: Followed the general procedure E using tert-butyl 2-hydroxy-8-azaspiro[4.5]decane-8-carboxylate (100 mg, 0.392 mmol, 1.0 equiv.) and 4-chloro-3-(trifluoromethyl)pyridine (60 mg, 0.392 mmol, 1.0 equiv.) as the starting materials to give tert-butyl 2-((3-(trifluoromethyl)pyridin-4-yl)oxy)-8-azaspiro[4.5]decane-8-carboxylate (80 mg, 51%) as a white solid. MS m/z: 401 [M+H]+.

Step 2: 2-((3-(trifluoromethyl)pyridin-4-yl)oxy)-8-azaspiro[4.5]decane hydrochloride: Followed the general procedure B using tert-butyl 2-((3-(trifluoromethyl)pyridin-4-yl)oxy)-8-azaspiro[4.5]decane-8-carboxylate (80 mg) as the starting material to give the crude product 2-((3-(trifluoromethyl)pyridin-4-yl)oxy)-8-azaspiro[4.5]decane hydrochloride (55 mg). MS m/z: 301 [M+H]⁺.

Step 3: 8-(1-(2,2-difluoroethyl)-1H-pyrazolo[3,4-b]pyrazin-6-yl)-2-((3-(trifluoromethyl)pyridin-4-yl)oxy)-8-azaspiro[4.5]decane: Followed the general procedure I using 2-((3-(trifluoromethyl)pyridin-4-yl)oxy)-8-azaspiro[4.5]decane hydrochloride (55 mg, 0.183 mmol, 1.0 equiv.) and 6-chloro-1-(2,2-difluoroethyl)-1H-pyrazolo[3,4-b]pyrazine (40 mg, 0.183 mmol, 1.0 equiv.) as the starting materials to give 8-(1-(2,2-difluoroethyl)-1H-pyrazolo[3,4-b]pyrazin-6-yl)-2-((3-(trifluoromethyl)pyridin-4-yl)oxy)-8-azaspiro[4.5]decane (43 mg, 49%) as a white solid. ¹H NMR (400 MHz, DMSO-d₆) δ 8.68 (t, J=3.0 Hz, 2H), 8.46 (s, 1H), 8.11 (s, 1H), 7.33 (d, J=5.9 Hz, 1H), 6.43 (tt, J=55.0, 3.9 Hz, 1H), 5.20 (tt, J=5.8, 2.5 Hz, 1H), 4.75-4.62 (m, 2H), 3.86-3.64 (m, 4H), 2.26-2.12 (m, 1H), 2.03 (dd, J=14.4, 6.2 Hz, 1H), 1.90-1.73 (m, 2H), 1.71-1.50 (m, 6H). MS m/z: 483.2 [M+H]⁺.

### Example 688: 8-(1-(2,2-difluoroethyl)-1H-pyrazolo[3,4-b]pyrazin-6-yl)-2-((4-(trifluoromethyl)pyridin-3-yl)oxy)-8-azaspiro[4.5]decane

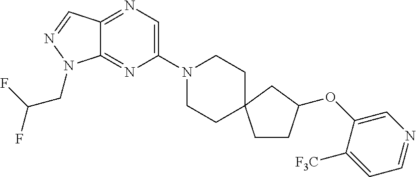 [SMILES: FC(F)Cn1ncc2ncc(N3CCC4(CCC(Oc5cnccc5C(F)(F)F)C4)CC3)nc21]

Step 1: tert-butyl 2-((4-(trifluoromethyl)pyridin-3-yl)oxy)-8-azaspiro[4.5]decane-8-carboxylate: Followed the general procedure C using 4-(trifluoromethyl)pyridin-3-ol (100 mg, 613 μmol, 1.0 equiv.) and tert-butyl 2-hydroxy-8-azaspiro[4.5]decane-8-carboxylate (156 mg, 611 μmol, 1.0 equiv.) as the starting materials to give tert-butyl 2-((4-(trifluoromethyl)pyridin-3-yl)oxy)-8-azaspiro[4.5]decane-8-carboxylate (100 mg, 40%) as a yellow oil. MS m/z: 401 [M+H]⁺.

Step 2: 2-((4-(trifluoromethyl)pyridin-3-yl)oxy)-8-azaspiro[4.5]decane hydrochloride: Followed the general procedure B using tert-butyl 2-((4-(trifluoromethyl)pyridin-3-yl)oxy)-8-azaspiro[4.5]decane-8-carboxylate (100 mg) as the starting material to give the crude product 2-((4-(trifluoromethyl)pyridin-3-yl)oxy)-8-azaspiro[4.5]decane hydrochloride (80 mg) as a yellow solid. MS m/z: 301 [M+H]⁺

Step 3: 8-(1-(2,2-difluoroethyl)-1H-pyrazolo[3,4-b]pyrazin-6-yl)-2-((4-(trifluoromethyl)pyridin-3-yl)oxy)-8-azaspiro[4.5]decane: Followed the general procedure I using 2-((4-(trifluoromethyl)pyridin-3-yl)oxy)-8-azaspiro[4.5]decane hydrochloride (70 mg, 233 μmol, 1.0 equiv.) and 6-chloro-1-(2,2-difluoroethyl)-1H-pyrazolo[3,4-b]pyrazine (50.8 mg, 233 μmol, 1.0 equiv.) as the starting materials to give 8-(1-(2,2-difluoroethyl)-1H-pyrazolo[3,4-b]pyrazin-6-yl)-2-((4-(trifluoromethyl)pyridin-3-yl)oxy)-8-azaspiro[4.5]decane (26.9 mg, 24%) as a white solid. ¹H NMR (400 MHz, DMSO-d₆) δ 8.69 (s, 1H), 8.46 (s, 1H), 8.39 (d, J=4.9 Hz, 1H), 8.11 (s, 1H), 7.64 (d, J=4.9 Hz, 1H), 6.43 (tt, J=54.9, 3.8 Hz, 1H), 5.31-5.23 (m, 1H), 4.68 (td, J=15.0, 3.8 Hz, 2H), 3.84-3.67 (m, 4H), 2.25-2.15 (m, 1H), 2.10-2.02 (m, 1H), 1.93-1.84 (m, 1H), 1.82-1.78 (m, 1H), 1.75-1.69 (m, 1H), 1.67-1.62 (m, 3H), 1.60-1.53 (m, 2H). MS m/z: 483.2 [M+H]⁺.

### Example 689: 8-(1-(2,2-difluoroethyl)-1H-pyrazolo[3,4-b]pyrazin-6-yl)-2-((3-(trifluoromethyl)pyridin-2-yl)oxy)-8-azaspiro[4.5]decane

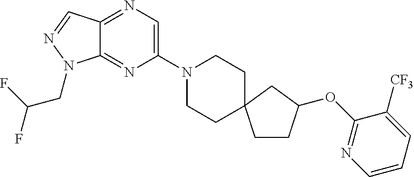 [SMILES: FC(F)Cn1ncc2ncc(N3CCC4(CCC(Oc5ncccc5C(F)(F)F)C4)CC3)nc21]

Step 1: 8-(1-(2,2-difluoroethyl)-1H-pyrazolo[3,4-b]pyrazin-6-yl)-2-((3-(trifluoromethyl)pyridin-2-yl)oxy)-8-azaspiro[4.5]decane: Followed the general procedure I using 2-((3-(trifluoromethyl)pyridin-2-yl)oxy)-8-azaspiro[4.5]decane hydrochloride (60 mg, 0.202 mmol, 1.0 equiv.) and 6-chloro-1-(2,2-difluoroethyl)pyrazolo[3,4-b]pyrazine (44 mg, 0.202 mmol, 1 equiv.) as the starting materials to give 8-(1-(2,2-difluoroethyl)-1H-pyrazolo[3,4-b]pyrazin-6-yl)-2-((3-(trifluoromethyl)pyridin-2-yl)oxy)-8-azaspiro[4.5]decane (80 mg, 82%) as a white solid. ¹H NMR (400 MHz, DMSO-d₆) δ 8.46-8.43 (m, 2H), 8.11-8.07 (m, 2H), 7.14 (d, 1H), 6.42 (tt, 1H), 5.68-5.52 (m, 1H), 4.69 (td, 2H), 3.82-3.69 (m, 4H), 2.18-1.97 (m, 2H), 1.90-1.70 (m, 3H), 1.69-1.60 (m, 3H), 1.59-1.53 (m, 2H). MS m/z: 483.20 [M+H]⁺.

### Example 690: 8-(1-(2,2-difluoroethyl)-1H-pyrazolo[3,4-b]pyrazin-6-yl)-2-((2-(trifluoromethyl)pyridin-4-yl)oxy)-8-azaspiro[4.5]decane

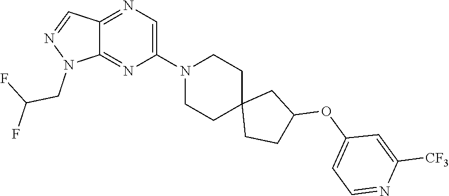 [SMILES: FC(F)Cn1ncc2ncc(N3CCC4(CCC(Oc5ccnc(C(F)(F)F)c5)C4)CC3)nc21]

Step 1: tert-butyl 2-((2-(trifluoromethyl)pyridin-4-yl)oxy)-8-azaspiro[4.5]decane-8-carboxylate: Followed the general procedure E using tert-butyl 2-hydroxy-8-azaspiro[4.5]decane-8-carboxylate (150 mg, 0.586 mmol, 1.0 equiv.) and 4-chloro-2-(trifluoromethyl)pyridine (128 mg, 0.703 mmol, 1.2 equiv.) as the starting materials to give tert-butyl 2-((2-(trifluoromethyl)pyridin-4-yl)oxy)-8-azaspiro[4.5]decane-8-carboxylate (150 mg, 63.8%) as a light yellow oil. MS m/z: 401 [M+H]⁺.

Step 2: 2-((2-(trifluoromethyl)pyridin-4-yl)oxy)-8-azaspiro[4.5]decane hydrochloride: Followed the general procedure B using tert-butyl 2-((2-(trifluoromethyl)pyridin-4-yl)oxy)-8-azaspiro[4.5]decane-8-carboxylate (150 mg, 0.374 mmol, 1.0 equiv.) as the starting material to give the crude product 2-((2-(trifluoromethyl)pyridin-4-yl)oxy)-8-azaspiro[4.5]decane hydrochloride (120 mg). MS m/z: 301 [M+H]⁺.

Step 3: 8-(1-(2,2-difluoroethyl)-1H-pyrazolo[3,4-b]pyrazin-6-yl)-2-((2-(trifluoromethyl)pyridin-4-yl)oxy)-8-azaspiro[4.5]decane: Followed the general procedure I using 2-((2-(trifluoromethyl)pyridin-4-yl)oxy)-8-azaspiro[4.5]decane hydrochloride (120 mg, 0.399 mmol, 1.0 equiv.) and 6-chloro-1-(2,2-difluoroethyl)-1H-pyrazolo[3,4-b]pyrazine (87.3 mg, 0.399 mmol, 1.0 equiv.) as the starting materials to give 8-(1-(2,2-difluoroethyl)-1H-pyrazolo[3,4-b]pyrazin-6-yl)-2-((2-(trifluoromethyl)pyridin-4-yl)oxy)-8-azaspiro[4.5]decane (66.2 mg, 34.3%) as a white solid. ¹H NMR (400 MHz, DMSO-d₆) δ 8.55 (d, J=5.7 Hz, 1H), 8.46 (s, 1H), 8.11 (s, 1H), 7.38 (d, J=2.4 Hz, 1H), 7.26 (dd, J=5.7, 2.4 Hz, 1H), 6.43 (tt, J=54.9, 3.8 Hz, 1H), 5.15 (tt, J=6.6, 3.4 Hz, 1H), 4.68 (td, J=15.0, 3.8 Hz, 2H), 3.88-3.66 (m, 4H), 2.28-2.16 (m, 1H), 2.13-2.05 (m, 1H), 1.89-1.79 (m, 1H), 1.78-1.69 (m, 2H), 1.65 (q, J=7.4, 6.4 Hz, 3H), 1.57 (q, J=5.7 Hz, 2H). MS m/z: 483.25 [M+H]⁺.

### Example 691: 8-(1-(2,2-difluoroethyl)-1H-pyrazolo[3,4-b]pyrazin-6-yl)-2-((5-(trifluoromethyl)pyridin-3-yl)oxy)-8-azaspiro[4.5]decane

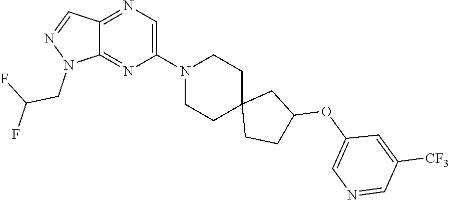 [SMILES: FC(F)Cn1ncc2ncc(N3CCC4(CCC(Oc5cncc(C(F)(F)F)c5)C4)CC3)nc21]

Step 1: tert-butyl 2-((5-(trifluoromethyl)pyridin-3-yl)oxy)-8-azaspiro[4.5]decane-8-carboxylate: Followed the general procedure C using tert-butyl 2-hydroxy-8-azaspiro[4.5]decane-8-carboxylate (250 mg, 981 μmol, 1.0 equiv.) and 5-(trifluoromethyl)pyridin-3-ol (160 mg, 981 μmol, 1.0 equiv.) as the starting materials to give tert-butyl 2-((5-(trifluoromethyl)pyridin-3-yl)oxy)-8-azaspiro[4.5]decane-8-carboxylate (354 mg, 90%). MS m/z: 401 [M+H]⁺.

Step 2: 2-((5-(trifluoromethyl)pyridin-3-yl)oxy)-8-azaspiro[4.5]decane hydrochloride: Followed the general procedure B using tert-butyl 2-((5-(trifluoromethyl)pyridin-3-yl)oxy)-8-azaspiro[4.5]decane-8-carboxylate (354 mg) as the starting material to give the crude product 2-((5-(trifluoromethyl)pyridin-3-yl)oxy)-8-azaspiro[4.5]decane hydrochloride (354 mg). MS m/z: 301 [M+H]⁺.

Step 3: 8-(1-(2,2-difluoroethyl)-1H-pyrazolo[3,4-b]pyrazin-6-yl)-2-((5-(trifluoromethyl)pyridin-3-yl)oxy)-8-azaspiro[4.5]decane: Followed the general procedure I using 2-((5-(trifluoromethyl)pyridin-3-yl)oxy)-8-azaspiro[4.5]decane hydrochloride (111 mg, 329 μmol, 1.2 equiv.) and 6-chloro-1-(2,2-difluoroethyl)-1H-pyrazolo[3,4-b]pyrazine (60 mg, 274 μmol, 1.0 equiv.) as the starting materials to give 8-(1-(2,2-difluoroethyl)-1H-pyrazolo[3,4-b]pyrazin-6-yl)-2-((5-(trifluoromethyl)pyridin-3-yl)oxy)-8-azaspiro[4.5]decane (95 mg, 70%) as a white solid. ¹H NMR (400 MHz, DMSO-d₆) δ 8.60-8.51 (m, 2H), 8.46 (s, 1H), 8.11 (d, J=0.7 Hz, 1H), 7.72 (t, J=2.4 Hz, 1H), 6.43 (tt, J=54.9, 3.8 Hz, 1H), 5.14 (tt, J=6.6, 3.5 Hz, 1H), 4.68 (td, J=15.0, 3.8 Hz, 2H), 3.75 (tt, J=13.7, 6.9 Hz, 4H), 2.27-2.13 (m, 1H), 2.08 (dd, J=14.0, 6.5 Hz, 1H), 1.83 (dd, J=13.4, 4.5 Hz, 1H), 1.74 (dt, J=10.1, 4.8 Hz, 2H), 1.69-1.61 (m, 3H), 1.57 (q, J=5.7 Hz, 2H). MS m/z: 483.2 [M+H]⁺.

### Example 692: 8-(1-(2,2-difluoroethyl)-1H-pyrazolo[3,4-b]pyrazin-6-yl)-2-((4-(trifluoromethyl)pyridin-2-yl)oxy)-8-azaspiro[4.5]decane

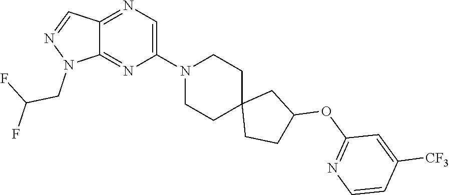 [SMILES: FC(F)Cn1ncc2ncc(N3CCC4(CCC(Oc5cc(C(F)(F)F)ccn5)C4)CC3)nc21]

Step 1: tert-butyl 2-((4-(trifluoromethyl)pyridin-2-yl)oxy)-8-azaspiro[4.5]decane-8-carboxylate: Followed the general procedure E using tert-butyl 2-hydroxy-8-azaspiro[4.5]decane-8-carboxylate (120 mg, 0.470 mmol, 1 equiv) and 2-fluoro-4-(trifluoromethyl)pyridine (85.34 mg, 0.517 mmol, 1.1 equiv) as the starting materials to give tert-butyl 2-((4-(trifluoromethyl)pyridin-2-yl)oxy)-8-azaspiro[4.5]decane-8-carboxylate (80 mg, 42%) as a white solid. MS m/z: 401 [M+H]⁺.

Step 2: 2-((4-(trifluoromethyl)pyridin-2-yl)oxy)-8-azaspiro[4.5]decane hydrochloride: Followed the general procedure B using tert-butyl 2-((4-(trifluoromethyl)pyridin-2-yl)oxy)-8-azaspiro[4.5]decane-8-carboxylate (200 mg, 0.485 mmol, 1 equiv) as the starting material to give the crude product 2-((4-(trifluoromethyl)pyridin-2-yl)oxy)-8-azaspiro[4.5]decane hydrochloride (150 mg). MS m/z: 301 [M+H]⁺.

Step 3: 8-(1-(2,2-difluoroethyl)-1H-pyrazolo[3,4-b]pyrazin-6-yl)-2-((4-(trifluoromethyl)pyridin-2-yl)oxy)-8-azaspiro[4.5]decane: Followed the general procedure I using 2-((4-(trifluoromethyl)pyridin-2-yl)oxy)-8-azaspiro[4.5]decane hydrochloride (80 mg, 0.266 mmol, 1 equiv) and 6-chloro-1-(2,2-difluoroethyl)-1H-pyrazolo[3,4-b]pyrazine (58.23 mg, 0.266 mmol, 1 equiv) as the starting materials to give 8-(1-(2,2-difluoroethyl)-1H-pyrazolo[3,4-b]pyrazin-6-yl)-2-((4-(trifluoromethyl)pyridin-2-yl)oxy)-8-azaspiro[4.5]decane (60 mg, 45%) as a white solid. ¹H NMR (400 MHz, DMSO-d₆) δ 8.49-8.40 (m, 2H), 8.11 (s, 1H), 7.29 (dd, J=5.4, 1.5 Hz, 1H), 7.14 (s, 1H), 6.43 (tt, J=54.9, 3.8 Hz, 1H), 5.53-5.43 (m, 1H), 4.68 (td, J=15.0, 3.8 Hz, 2H), 3.84-3.66 (m, 4H), 2.24-2.12 (m, 1H), 2.12-2.02 (m, 1H), 1.89-1.81 (m, 1H), 1.79-1.62 (m, 5H), 1.60-1.52 (m, 2H). MS m/z: 483.4 [M+H]⁺.

### Example 693: 8-(1-(2,2-difluoroethyl)-1H-pyrazolo[3,4-b]pyrazin-6-yl)-2-((6-(trifluoromethyl)pyridin-3-yl)oxy)-8-azaspiro[4.5]decane

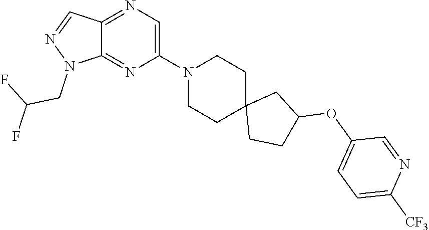 [SMILES: FC(F)Cn1ncc2ncc(N3CCC4(CCC(Oc5ccc(C(F)(F)F)nc5)C4)CC3)nc21]

Step 1: tert-butyl 2-((6-(trifluoromethyl)pyridin-3-yl)oxy)-8-azaspiro[4.5]decane-8-carboxylate: Followed the general procedure E using tert-butyl 2-hydroxy-8-azaspiro[4.5]decane-8-carboxylate (90 mg, 0.352 mmol, 1.0 equiv.) and 5-fluoro-2-(trifluoromethyl)pyridine (70.1 mg, 0.422 mmol, 1.2 equiv.) as the starting materials to give tert-butyl 2-((6-(trifluoromethyl)pyridin-3-yl)oxy)-8-azaspiro[4.5]decane-8-carboxylate (100 mg, 63%) as a white solid. MS m/z: 401 [M+H]⁺.

Step 2: 2-((6-(trifluoromethyl)pyridin-3-yl)oxy)-8-azaspiro[4.5]decane hydrochloride: Followed the general procedure B using tert-butyl 2-((6-(trifluoromethyl)pyridin-3-yl)oxy)-8-azaspiro[4.5]decane-8-carboxylate (100 mg) as the starting material to give the crude product 2-((6-(trifluoromethyl)pyridin-3-yl)oxy)-8-azaspiro[4.5]decane hydrochloride (70 mg). MS m/z: 301 [M+H]⁺.

Step 3: 8-(1-(2,2-difluoroethyl)-1H-pyrazolo[3,4-b]pyrazin-6-yl)-2-((6-(trifluoromethyl)pyridin-3-yl)oxy)-8-azaspiro[4.5]decane: Followed the general procedure I using 2-((6-(trifluoromethyl)pyridin-3-yl)oxy)-8-azaspiro[4.5]decane hydrochloride (70 mg, 0.233 mmol, 1.0 equiv.) and 6-chloro-1-(2,2-difluoroethyl)-1H-pyrazolo[3,4-b]pyrazine (56.1 mg, 0.256 mmol, 1.1 equiv.) as the starting materials to give 8-(1-(2,2-difluoroethyl)-1H-pyrazolo[3,4-b]pyrazin-6-yl)-2-((6-(trifluoromethyl)pyridin-3-yl)oxy)-8-azaspiro[4.5]decane (86.9 mg, 77%) as a white solid. ¹H NMR (400 MHz, DMSO-d₆) δ 8.48-8.39 (m, 2H), 8.11 (s, 1H), 7.83 (d, J=8.8 Hz, 1H), 7.63-7.55 (m, 1H), 6.61-6.24 (m, 1H), 5.09 (s, 1H), 4.75-4.62 (m, 2H), 3.82-3.67 (m, 4H), 2.26-2.16 (m, 1H), 2.14-2.05 (m, 1H), 1.87 (s, 1H), 1.78-1.71 (m, 2H), 1.65 (t, J=5.8 Hz, 3H), 1.57 (d, J=5.6 Hz, 2H). MS m/z: 483.3 [M+H]⁺.

### Example 694: 8-(1-(2,2-difluoroethyl)-1H-pyrazolo[3,4-b]pyrazin-6-yl)-2-((5-(trifluoromethyl)pyridin-2-yl)oxy)-8-azaspiro[4.5]decane

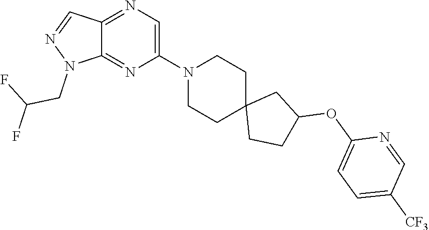 [SMILES: FC(F)Cn1ncc2ncc(N3CCC4(CCC(Oc5ccc(C(F)(F)F)cn5)C4)CC3)nc21]

Step 1: tert-butyl 2-((5-(trifluoromethyl)pyridin-2-yl)oxy)-8-azaspiro[4.5]decane-8-carboxylate: Followed the general procedure I using tert-butyl 2-hydroxy-8-azaspiro[4.5]decane-8-carboxylate (150 mg, 0.587 mmol, 1.0 equiv.) and 2-fluoro-5-(trifluoromethyl)pyridine (116 mg, 0.704 mmol, 1.2 equiv.) as the starting materials to give tert-butyl 2-((5-(trifluoromethyl)pyridin-2-yl)oxy)-8-azaspiro[4.5]decane-8-carboxylate (210 mg, 89%) as a white solid. MS m/z: 401 [M+H]⁺.

Step 2: 2-((5-(trifluoromethyl)pyridin-2-yl)oxy)-8-azaspiro[4.5]decane hydrochloride: Followed the general procedure B using tert-butyl 2-((5-(trifluoromethyl)pyridin-2-yl)oxy)-8-azaspiro[4.5]decane-8-carboxylate (210 mg) as the starting material to give the crude product 2-((5-(trifluoromethyl)pyridin-2-yl)oxy)-8-azaspiro[4.5]decane hydrochloride (210 mg). MS m/z: 301 [M+H]⁺.

Step 3: 8-(1-(2,2-difluoroethyl)-1H-pyrazolo[3,4-b]pyrazin-6-yl)-2-((5-(trifluoromethyl)pyridin-2-yl)oxy)-8-azaspiro[4.5]decane: Followed the general procedure I using 2-((5-(trifluoromethyl)pyridin-2-yl)oxy)-8-azaspiro[4.5]decane hydrochloride (92.4 mg, 0.274 mmol, 1.0 equiv.) and 6-chloro-1-(2,2-difluoroethyl)-1H-pyrazolo[3,4-b]pyrazine (60 mg, 0.274 mmol, 1.0 equiv.) as the starting materials to give 8-(1-(2,2-difluoroethyl)-1H-pyrazolo[3,4-b]pyrazin-6-yl)-2-((5-(trifluoromethyl)pyridin-2-yl)oxy)-8-azaspiro[4.5]decane (86.8 mg, 65%) as a yellow oil. ¹H NMR (400 MHz, DMSO-d₆) δ 8.61-8.55 (m, 1H), 8.46 (s, 1H), 8.14-8.00 (m, 2H), 6.98 (d, J=8.7 Hz, 1H), 6.43 (tt, J=54.9, 3.8 Hz, 1H), 5.54-5.44 (m, 1H), 4.68 (td, J=15.0, 3.8 Hz, 2H), 3.84-3.66 (m, 4H), 2.24-2.11 (m, 1H), 2.08 (dd, J=14.1, 6.7 Hz, 1H), 1.90-1.81 (m, 1H), 1.79-1.60 (m, 5H), 1.58-1.54 (m, 2H). MS m/z: 483.3 [M+H]⁺.

### Example 695: 1-(2,2-difluoroethyl)-6-(2-(((2-(trifluoromethyl)pyridin-4-yl)oxy)methyl)-7-azaspiro[3.5]nonan-7-yl)-1H-pyrazolo[3,4-b]pyrazine

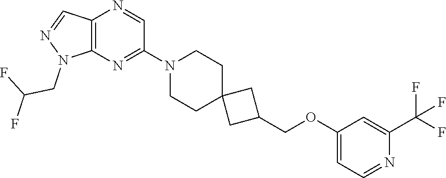 [SMILES: FC(F)Cn1ncc2ncc(N3CCC4(CC3)CC(COc3ccnc(C(F)(F)F)c3)C4)nc21]

Step 1: tert-butyl 2-(((2-(trifluoromethyl)pyridin-4-yl)oxy)methyl)-7-azaspiro[3.5]nonane-7-carboxylate: Followed the general procedure E using tert-butyl 2-(hydroxymethyl)-7-azaspiro[3.5]nonane-7-carboxylate (150 mg, 0.587 mmol, 1.0 equiv.) and 4-chloro-2-(trifluoromethyl)pyridine (160 mg, 0.880 mmol, 1.5 equiv.) as the starting materials to give tert-butyl 2-(((2-(trifluoromethyl)pyridin-4-yl)oxy)methyl)-7-azaspiro[3.5]nonane-7-carboxylate (150 mg, 63.8%) as a light yellow oil. MS m/z: 401 [M+H]⁺.

Step 2: 2-(((2-(trifluoromethyl)pyridin-4-yl)oxy)methyl)-7-azaspiro[3.5]nonane hydrochloride: Followed the general procedure B using tert-butyl 2-(((2-(trifluoromethyl)pyridin-4-yl)oxy)methyl)-7-azaspiro[3.5]nonane-7-carboxylate (150 mg, 0.374 mmol, 1.0 equiv.) as the starting material to give the crude product 2-(((2-(trifluoromethyl)pyridin-4-yl)oxy)methyl)-7-azaspiro[3.5]nonane hydrochloride (100 mg). MS m/z: 301 [M+H]⁺.

Step 3: 1-(2,2-difluoroethyl)-6-(2-(((2-(trifluoromethyl)pyridin-4-yl)oxy)methyl)-7-azaspiro[3.5]nonan-7-yl)-1H-pyrazolo[3,4-b]pyrazine: Followed the general procedure I using 2-(((2-(trifluoromethyl)pyridin-4-yl)oxy)methyl)-7-azaspiro[3.5]nonane hydrochloride (92.4 mg, 0.274 mmol, 1.0 equiv.) and 6-chloro-1-(2,2-difluoroethyl)-1H-pyrazolo[3,4-b]pyrazine (60.0 mg, 0.274 mmol, 1.0 equiv.) as the starting materials to give 1-(2,2-difluoroethyl)-6-(2-(((2-(trifluoromethyl)pyridin-4-yl)oxy)methyl)-7-azaspiro[3.5]nonan-7-yl)-1H-pyrazolo[3,4-b]pyrazine (68.5 mg, 51.2%) as a white solid. ¹H NMR (400 MHz, DMSO-d₆) δ 8.56 (d, J=5.7 Hz, 1H), 8.46 (s, 1H), 8.11 (s, 1H), 7.44 (d, J=2.4 Hz, 1H), 7.28 (dd, J=5.8, 2.5 Hz, 1H), 6.43 (tt, J=54.8, 3.8 Hz, 1H), 4.68 (td, J=15.0, 3.8 Hz, 2H), 4.20 (d, J=6.6 Hz, 2H), 3.74 (t, J=5.6 Hz, 2H), 3.65 (t, J=5.6 Hz, 2H), 2.84-2.68 (m, 1H), 2.10-1.96 (m, 2H), 1.76-1.52 (m, 6H). MS m/z: 483.3 [M+H]⁺.

### Example 696: 1-(2,2-difluoroethyl)-6-(2-(((5-(trifluoromethyl)pyridin-3-yl)oxy)methyl)-7-azaspiro[3.5]nonan-7-yl)-1H-pyrazolo[3,4-b]pyrazine

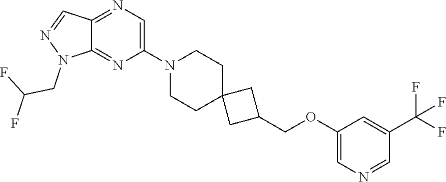 [SMILES: FC(F)Cn1ncc2ncc(N3CCC4(CC3)CC(COc3cncc(C(F)(F)F)c3)C4)nc21]

Step 1. tert-butyl 2-(((5-(trifluoromethyl)pyridin-3-yl)oxy)methyl)-7-azaspiro[3.5]nonane-7-carboxylate: Followed the general procedure X using tert-butyl 2-(hydroxymethyl)-7-azaspiro[3.5]nonane-7-carboxylate (200 mg, 1.29 mmol, 1.0 equiv.) and 3-fluoro-5-(trifluoromethyl)pyridine (274 mg, 2.59 mmol, 2.0 equiv.) as the starting materials to give tert-butyl 2-((5-(trifluoromethyl)pyridin-3-yl)oxy)-7-azaspiro[3.5]nonane-7-carboxylate (140 mg, 55%) as a white solid. MS m/z: 401 [M+H]⁺

Step 2. 2-(((5-(trifluoromethyl)pyridin-3-yl)oxy)methyl)-7-azaspiro[3.5]nonane hydrochloride: Followed the general procedure B using tert-butyl 2-((5-(trifluoromethyl)pyridin-3-yl)oxy)-7-azaspiro[3.5]nonane-7-carboxylate (130 mg) as the starting material to give the crude product 2-((5-(trifluoromethyl)pyridin-3-yl)oxy)-7-azaspiro[3.5]nonane hydrochloride (100 mg). MS m/z: 301 [M+H]⁺.

Step 3. 1-(2,2-difluoroethyl)-6-(2-(((5-(trifluoromethyl)pyridin-3-yl)oxy)methyl)-7-azaspiro[3.5]nonan-7-yl)-1H-pyrazolo[3,4-b]pyrazine: Followed the general procedure I using 2-((5-(trifluoromethyl)pyridin-3-yl)oxy)-7-azaspiro[3.5]nonane hydrochloride (90 mg, 0.238 mmol, 1.0 equiv.) and 6-chloro-1-(2,2-difluoroethyl)-1H-pyrazolo[3,4-b]pyrazine (52 mg, 0.238 mmol, 1.0 equiv.) as the starting materials to give 1-(2,2-difluoroethyl)-6-(2-(((5-(trifluoromethyl)pyridin-3-yl)oxy)methyl)-7-azaspiro[3.5]nonan-7-yl)-1H-pyrazolo[3,4-b]pyrazine (60 mg, 90%) as a white solid. ¹H NMR (400 MHz, DMSO-d₆) δ 8.60 (d, J=2.7 Hz, 1H), 8.57-8.52 (m, 1H), 8.47 (s, 1H), 8.11 (d, J=0.9 Hz, 1H), 7.77 (t, J=2.4 Hz, 1H), 6.43 (tt, J=55.0, 3.9 Hz, 1H), 4.75-4.62 (m, 2H), 4.18 (d, J=6.6 Hz, 2H), 3.74 (t, J=5.6 Hz, 2H), 3.66 (t, J=5.6 Hz, 2H), 2.83-2.69 (m, 1H), 2.10-1.97 (m, 2H), 1.76-1.67 (m, 4H), 1.63 (t, J=5.7 Hz, 2H). MS m/z: 483.2 [M+H]⁺.

### Example 697: 1-(2,2-difluoroethyl)-6-(2-(((4-(trifluoromethyl)pyridin-2-yl)oxy)methyl)-7-azaspiro[3.5]nonan-7-yl)-1H-pyrazolo[3,4-b]pyrazine

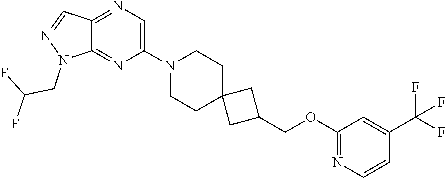 [SMILES: FC(F)Cn1ncc2ncc(N3CCC4(CC3)CC(COc3cc(C(F)(F)F)ccn3)C4)nc21]

Step 1: tert-butyl 2-(((4-(trifluoromethyl)pyridin-2-yl)oxy)methyl)-7-azaspiro[3.5]nonane-7-carboxylate: Followed the general procedure E using tert-butyl 2-(hydroxymethyl)-7-azaspiro[3.5]nonane-7-carboxylate (120 mg, 0.470 mmol, 1 equiv) and 2-fluoro-4-(trifluoromethyl)pyridine (85.34 mg, 0.517 mmol, 1.1 equiv) as the starting materials to give tert-butyl 2-(((4-(trifluoromethyl)pyridin-2-yl)oxy)methyl)-7-azaspiro[3.5]nonane-7-carboxylate (100 mg, 42.5%) as a white solid. MS m/z: 401 [M+H]⁺.

Step 2: 2-(((4-(trifluoromethyl)pyridin-2-yl)oxy)methyl)-7-azaspiro[3.5]nonane hydrochloride: Followed the general procedure B using tert-butyl 2-(((4-(trifluoromethyl)pyridin-2-yl)oxy)methyl)-7-azaspiro[3.5]nonane-7-carboxylate (110 mg, 0.275 mmol, 1 equiv) as the starting material to give the crude product 2-(((4-(trifluoromethyl)pyridin-2-yl)oxy)methyl)-7-azaspiro[3.5]nonane hydrochloride (80 mg). MS m/z: 301 [M+H]⁺.

Step 3: 8-(1-(2,2-difluoroethyl)-1H-pyrazolo[3,4-b]pyrazin-6-yl)-2-((4-(trifluoromethyl)pyridin-2-yl)oxy)-8-azaspiro[4.5]decane: Followed the general procedure I using 2-(((4-(trifluoromethyl)pyridin-2-yl)oxy)methyl)-7-azaspiro[3.5]nonane hydrochloride (80 mg, 0.266 mmol, 1 equiv) and 6-chloro-1-(2,2-difluoroethyl)-1H-pyrazolo[3,4-b]pyrazine (58.23 mg, 0.266 mmol, 1 equiv) as the starting materials to give 1-(2,2-difluoroethyl)-6-(2-(((4-(trifluoromethyl)pyridin-2-yl)oxy)methyl)-7-azaspiro[3.5]nonan-7-yl)-1H-pyrazolo[3,4-b]pyrazine (60 mg, 44%) as a white solid. ¹H NMR (400 MHz, DMSO-d₆) δ 8.48-8.40 (m, 2H), 8.11 (s, 1H), 7.32 (d, J=5.3 Hz, 1H), 7.19 (s, 1H), 6.43 (tt, J=55.0, 3.9 Hz, 1H), 4.68 (td, J=15.0, 3.8 Hz, 2H), 4.35 (d, J=6.6 Hz, 2H), 3.69 (dt, J=32.8, 6.0 Hz, 4H), 2.82-2.70 (m, 1H), 2.04-1.95 (m, 2H), 1.73-1.65 (m, 4H), 1.65-1.58 (m, 2H). MS m/z: 483.4 [M+H]⁺.

### Example 698: 1-(2,2-difluoroethyl)-6-(2-(((3-(trifluoromethyl)pyridin-4-yl)oxy)methyl)-7-azaspiro[3.5]nonan-7-yl)-1H-pyrazolo[3,4-b]pyrazine

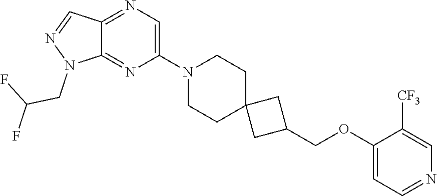 [SMILES: FC(F)Cn1ncc2ncc(N3CCC4(CC3)CC(COc3ccncc3C(F)(F)F)C4)nc21]

Step 1: tert-butyl 2-(((3-(trifluoromethyl)pyridin-4-yl)oxy)methyl)-7-azaspiro[3.5]nonane-7-carboxylate: Followed the general procedure E using tert-butyl 2-(hydroxymethyl)-7-azaspiro[3.5]nonane-7-carboxylate (100 mg, 0.392 mmol, 1.0 equiv.) and 4-chloro-3-(trifluoromethyl)pyridine (60 mg, 0.392 mmol, 1.0 equiv.) as the starting materials to give tert-butyl 2-(((3-(trifluoromethyl)pyridin-4-yl)oxy)methyl)-7-azaspiro[3.5]nonane-7-carboxylate (120 mg, 77%) as a white solid. MS m/z: 401 [M+H]⁺.

Step 2: 2-(((3-(trifluoromethyl)pyridin-4-yl)oxy)methyl)-7-azaspiro[3.5]nonane hydrochloride: Followed the general procedure B using tert-butyl 2-(((3-(trifluoromethyl)pyridin-4-yl)oxy)methyl)-7-azaspiro[3.5]nonane-7-carboxylate (120 mg) as the starting material to give the crude product 2-(((3-(trifluoromethyl)pyridin-4-yl)oxy)methyl)-7-azaspiro[3.5]nonane hydrochloride (90 mg). MS m/z: 301 [M+H]⁺.

Step 3: 1-(2,2-difluoroethyl)-6-(2-(((3-(trifluoromethyl)pyridin-oxy(methyl) 7-azaspiro[3.5]nonan-7-yl)-1H-pyrazolo[3,4-b]pyrazine: Followed the general procedure I using 2-(((3-(trifluoromethyl)pyridin-4-yl)oxy)methyl)-7-azaspiro[3.5]nonane hydrochloride (90 mg, 0.3 mmol, 1.0 equiv.) and 6-chloro-1-(2,2-difluoroethyl)-1H-pyrazolo[3,4-b]pyrazine (65 mg, 0.3 mmol, 1.0 equiv.) as the starting materials to give 1-(2,2-difluoroethyl)-6-(2-(((3-(trifluoromethyl)pyridin-4-yl)oxy)methyl)-7-azaspiro[3.5]nonan-7-yl)-1H-pyrazolo[3,4-b]pyrazine (60 mg, 42%) as a white solid. ¹H NMR (400 MHz, DMSO-d₆) δ 8.73-8.67 (m, 2H), 8.46 (s, 1H), 8.11 (s, 1H), 7.34 (d, J=5.9 Hz, 1H), 6.43 (tt, J=54.9, 3.9 Hz, 1H), 4.69 (td, J=15.0, 3.8 Hz, 2H), 4.22 (d, J=5.5 Hz, 2H), 3.83-3.56 (m, 4H), 2.86-2.74 (m, 1H), 2.01-1.91 (m, 2H), 1.83-1.66 (m, 4H), 1.63-1.56 (m, 2H). MS m/z: 483.2 [M+H]⁺.

### Example 699: 1-(2,2-difluoroethyl)-6-(2-(((4-(trifluoromethyl)pyridin-3-yl)oxy)methyl)-7-azaspiro[3.5]nonan-7-yl)-1H-pyrazolo[3,4-b]pyrazine

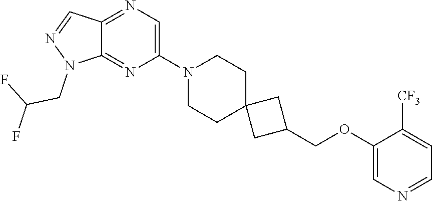 [SMILES: FC(F)Cn1ncc2ncc(N3CCC4(CC3)CC(COc3cnccc3C(F)(F)F)C4)nc21]

Step 1: tert-butyl 2-(((4-(trifluoromethyl)pyridin-3-yl)oxy)methyl)-7-azaspiro[3.5]nonane-7-carboxylate: Followed the general procedure C using 4-(trifluoromethyl)pyridin-3-ol (100 mg, 613 μmol, 1.0 equiv.) and tert-butyl 2-(hydroxymethyl)-7-azaspiro[3.5]nonane-7-carboxylate (156 mg, 613 μmol, 1.0 equiv.) as the starting materials to give tert-butyl 2-(((4-(trifluoromethyl)pyridin-3-yl)oxy)methyl)-7-azaspiro[3.5]nonane-7-carboxylate (80 mg, 32%) as a yellow oil. MS m/z: 401 [M+H]⁺.

Step 2: 2-(((4-(trifluoromethyl)pyridin-3-yl)oxy)methyl)-7-azaspiro[3.5]nonane hydrochloride: Followed the general procedure B using tert-butyl 2-(((4-(trifluoromethyl)pyridin-3-yl)oxy)methyl)-7-azaspiro[3.5]nonane-7-carboxylate (80 mg) as the starting material to give the crude product 2-(((4-(trifluoromethyl)pyridin-3-yl)oxy)methyl)-7-azaspiro[3.5]nonane hydrochloride (70 mg) as a yellow solid. MS m/z: 301 [M+H]⁺.

Step 3: 1-(2,2-difluoroethyl)-6-(2-(((4-(trifluoromethyl)pyridin-3-yl)oxy)methyl)-7-azaspiro[3.5]nonan-7-yl)-1H-pyrazolo[3,4-b]pyrazine: Followed the general procedure I using 2-(((4-(trifluoromethyl)pyridin-3-yl)oxy)methyl)-7-azaspiro[3.5]nonane hydrochloride (70 mg, 233 μmol, 1.0 equiv.) and 6-chloro-1-(2,2-difluoroethyl)-1H-pyrazolo[3,4-b]pyrazine (61 mg, 279 μmol, 1.2 equiv.) as the starting materials to give 1-(2,2-difluoroethyl)-6-(2-(((4-(trifluoromethyl)pyridin-3-yl)oxy)methyl)-7-azaspiro[3.5]nonan-7-yl)-1H-pyrazolo[3,4-b]pyrazine (31.9 mg, 28%) as a white solid. ¹H NMR (400 MHz, DMSO-d₆) δ 8.70 (s, 1H), 8.47 (s, 1H), 8.41 (d, J=4.9 Hz, 1H), 8.11 (s, 1H), 7.65 (d, J=4.9 Hz, 1H), 6.43 (tt, J=54.9, 3.8 Hz, 1H), 4.69 (td, J=15.0, 3.8 Hz, 2H), 4.27 (d, J=5.5 Hz, 2H), 3.80-3.60 (m, 4H), 2.86-2.74 (m, 1H), 2.00-1.93 (m, 2H), 1.81-1.70 (m, 4H), 1.63-1.56 (m, 2H). MS m/z: 483.2 [M+H]⁺.

### Example 700: 1-(2,2-difluoroethyl)-6-(2-(((3-(trifluoromethyl)pyridin-2-yl)oxy)methyl)-7-azaspiro[3.5]nonan-7-yl)-1H-pyrazolo[3,4-b]pyrazine

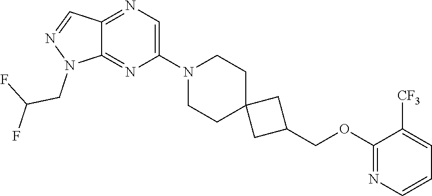 [SMILES: FC(F)Cn1ncc2ncc(N3CCC4(CC3)CC(COc3ncccc3C(F)(F)F)C4)nc21]

Step 1: 1-(2,2-difluoroethyl)-6-(2-(((3-(trifluoromethyl)pyridin-2-yl)oxy)methyl)-7-azaspiro[3.5]nonan-7-yl)-1H-pyrazolo[3,4-b]pyrazine: Followed the general procedure I using 2-(((3-(trifluoromethyl)pyridin-2-yl)oxy)methyl)-7-azaspiro[3.5]nonane (60 mg, 0.202 mmol, 1.0 equiv.) and 6-chloro-1-(2,2-difluoroethyl)pyrazolo[3,4-b]pyrazine (44 mg, 0.202 mmol, 1 equiv.) as the starting materials to give 1-(2,2-difluoroethyl)-6-(2-(((3-(trifluoromethyl)pyridin-2-yl)oxy)methyl)-7-azaspiro[3.5]nonan-7-yl)-1H-pyrazolo[3,4-b]pyrazine (39 mg, 40%) as a white solid. ¹H NMR (400 MHz, DMSO-d₆) δ 8.49-8.39 (m, 2H), 8.19-8.05 (m, 2H), 7.26-7.08 (m, 1H), 6.58-6.28 (m, 1H), 4.73-4.64 (m, 2H), 4.40-4.39 (m, 2H), 3.75-3.72 (m, 2H), 3.66-3.63 (m, 2H), 2.81-2.74 (m, 1H), 2.05-1.91 (m, 2H), 1.82-1.67 (m, 4H), 1.60-1.57 (m, 2H). MS m/z :483.4 [M+H]⁺.

### Example 701: 1-(2,2-difluoroethyl)-6-(2-(((6-(trifluoromethyl)pyridin-3-yl)oxy)methyl)-7-azaspiro[3.5]nonan-7-yl)-1H-pyrazolo[3,4-b]pyrazine

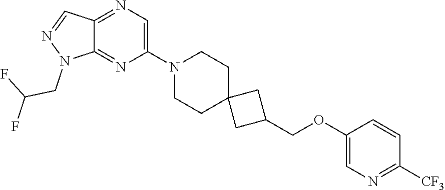 [SMILES: FC(F)Cn1ncc2ncc(N3CCC4(CC3)CC(COc3ccc(C(F)(F)F)nc3)C4)nc21]

Step 1: tert-butyl 2-(((6-(trifluoromethyl)pyridin-3-yl)oxy)methyl)-7-azaspiro[3.5]nonane-7-carboxylate: Followed the general procedure E using tert-butyl 3-oxo-2,8-diazaspiro[4.5]decane-8-carboxylate (100 mg, 0.391 mmol, 1.0 equiv.) and 5-fluoro-2-(trifluoromethyl)pyridine (77.8 mg, 0.469 mmol, 1.2 equiv.) as the starting materials to give tert-butyl 2-(((6-(trifluoromethyl)pyridin-3-yl)oxy)methyl)-7-azaspiro[3.5]nonane-7-carboxylate (70 mg, 45%) as a white solid. MS m/z: 401 [M+H]⁺.

Step 2: 2-(((6-(trifluoromethyl)pyridin-3-yl)oxy)methyl)-7-azaspiro[3.5]nonane hydrochloride: Followed the general procedure B using tert-butyl 2-(((6-(trifluoromethyl)pyridin-3-yl)oxy)methyl)-7-azaspiro[3.5]nonane-7-carboxylate (70 mg) as the starting material to give the crude product 2-(((6-(trifluoromethyl)pyridin-3-yl)oxy)methyl)-7-azaspiro[3.5]nonane hydrochloride (40 mg). MS m/z: 301 [M+H]⁺.

Step 3: 1-(2,2-difluoroethyl)-6-(2-(((6-(trifluoromethyl)pyridin-3-yl)oxy)methyl)-7-azaspiro[3.5]nonan-7-yl)-1H-pyrazolo[3,4-b]pyrazine: Followed the general procedure I using 2-(((6-(trifluoromethyl)pyridin-3-yl)oxy)methyl)-7-azaspiro[3.5]nonane hydrochloride (40 mg, 0.133 mmol, 1.0 equiv.) and 6-chloro-1-(2,2-difluoroethyl)-1H-pyrazolo[3,4-b]pyrazine (32.1 mg, 0.146 mmol, 1.1 equiv.) as the starting materials to give 1-(2,2-difluoroethyl)-6-(2-(((6-(trifluoromethyl)pyridin-3-yl)oxy)methyl)-7-azaspiro[3.5]nonan-7-yl)-1H-pyrazolo[3,4-b]pyrazine (17.9 mg, 32%) as a white solid. ¹H NMR (400 MHz, DMSO-d₆) δ 8.49-8.43 (m, 2H), 8.11 (s, 1H), 7.84 (d, J=8.7 Hz, 1H), 7.61 (d, J=11.2 Hz, 1H), 6.36 (d, J=54.9 Hz, 1H), 4.75-4.62 (m, 2H), 4.17 (d, J=6.6 Hz, 2H), 3.74 (s, 2H), 3.66 (s, 2H), 2.85-2.80 (m, 1H), 2.01 (d, J=10.6 Hz, 2H), 1.71 (s, 4H), 1.63 (s, 2H). MS m/z: 483.4 [M+H]⁺.

### Example 702: 1-(2,2-difluoroethyl)-6-(2-(((5-(trifluoromethyl)pyridin-2-yl)oxy)methyl)-7-azaspiro[3.5]nonan-7-yl)-1H-pyrazolo[3,4-b]pyrazine

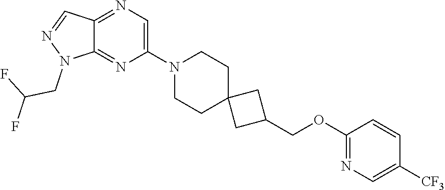 [SMILES: FC(F)Cn1ncc2ncc(N3CCC4(CC3)CC(COc3ccc(C(F)(F)F)cn3)C4)nc21]

Step 1: tert-butyl 2-(((5-(trifluoromethyl)pyridin-2-yl)oxy)methyl)-7-azaspiro[3.5]nonane-7-carboxylate: Followed the general procedure I using tert-butyl 2-(hydroxymethyl)-7-azaspiro[3.5]nonane-7-carboxylate (200 mg, 0.783 mmol, 1.0 equiv.) and 2-fluoro-5-(trifluoromethyl)pyridine (155 mg, 0.941 mmol, 1.2 equiv.) as the starting materials to give tert-butyl 2-(((5-(trifluoromethyl)pyridin-2-yl)oxy)methyl)-7-azaspiro[3.5]nonane-7-carboxylate (305 mg, 97%) as a yellow solid. MS m/z: 401 [M+H]⁺.

Step 2: 2-(((5-(trifluoromethyl)pyridin-2-yl)oxy)methyl)-7-azaspiro[3.5]nonane hydrochloride: Followed the general procedure B using tert-butyl 2-(((5-(trifluoromethyl)pyridin-2-yl)oxy)methyl)-7-azaspiro[3.5]nonane-7-carboxylate (305 mg) as the starting material to give the crude product 2-((5-(trifluoromethyl)pyridin-2-yl)oxy)-8-azaspiro[4.5]decane hydrochloride (305 mg). MS m/z: 301 [M+H]⁺.

Step 3: 1-(2,2-difluoroethyl)-6-(2-(((5-(trifluoromethyl)pyridin-2-yl)oxy)methyl)-7-azaspiro[3.5]nonan-7-yl)-1H-pyrazolo[3,4-b]pyrazine: Followed the general procedure I using 2-((5-(trifluoromethyl)pyridin-2-yl)oxy)-8-azaspiro[4.5]decane hydrochloride (92.4 mg, 0.274 mmol, 1.0 equiv.) and 6-chloro-1-(2,2-difluoroethyl)-1H-pyrazolo[3,4-b]pyrazine (60 mg, 0.274 mmol, 1.0 equiv.) as the starting materials to give 1-(2,2-difluoroethyl)-6-(2-(((5-(trifluoromethyl)pyridin-2-yl)oxy)methyl)-7-azaspiro[3.5]nonan-7-yl)-1H-pyrazolo[3,4-b]pyrazine (94.3 mg, 71%) as a white solid. ¹H NMR (400 MHz, DMSO-d₆) δ 8.58 (s, 1H), 8.46 (s, 1H), 8.14-8.02 (m, 2H), 7.02 (d, J=8.8 Hz, 1H), 6.43 (tt, J=54.9, 3.8 Hz, 1H), 4.68 (td, J=15.1, 3.8 Hz, 2H), 4.36 (d, J=6.9 Hz, 2H), 3.68 (dt, 4H), 2.84-2.68 (m, 1H), 2.00 (t, 2H), 1.73-1.64 (m, 4H), 1.64-1.57 (m, 2H). MS m/z: 483.3 [M+H]⁺.

### Example 703: 1-(2,2-difluoroethyl)-6-(2-((2-(trifluoromethyl)pyridin-4-yl)oxy)-7-azaspiro[3.5]nonan-7-yl)-1H-pyrazolo[3,4-b]pyrazine

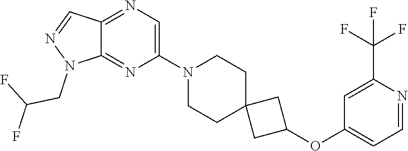 [SMILES: FC(F)Cn1ncc2ncc(N3CCC4(CC3)CC(Oc3ccnc(C(F)(F)F)c3)C4)nc21]

Step 1: tert-butyl 2-((2-(trifluoromethyl)pyridin-4-yl)oxy)-7-azaspiro[3.5]nonane-7-carboxylate: Followed the general procedure E using tert-butyl 2-hydroxy-7-azaspiro[3.5]nonane-7-carboxylate (150 mg, 0.620 mmol, 1.0 equiv.) and 4-chloro-2-(trifluoromethyl)pyridine (135 mg, 0.744 mmol, 1.2 equiv.) as the starting materials to give tert-butyl 2-((2-(trifluoromethyl)pyridin-4-yl)oxy)-7-azaspiro[3.5]nonane-7-carboxylate (150 mg, 62.5%) as a light yellow oil. MS m/z: 387 [M+H]⁺.

Step 2: 2-((2-(trifluoromethyl)pyridin-4-yl)oxy)-7-azaspiro[3.5]nonane hydrochloride: Followed the general procedure B using tert-butyl 2-((2-(trifluoromethyl)pyridin-4-yl)oxy)-7-azaspiro[3.5]nonane-7-carboxylate (150 mg, 0.388 mmol, 1.0 equiv.) as the starting material to give the crude product 2-((2-(trifluoromethyl)pyridin-4-yl)oxy)-7-azaspiro[3.5]nonane hydrochloride (120 mg). MS m/z: 287 [M+H]⁺.

Step 3: 1-(2,2-difluoroethyl)-6-(2-((2-(trifluoromethyl)pyridin-4-yl)oxy)-7-azaspiro[3.5]nonan-7-yl)-1H-pyrazolo[3,4-b]pyrazine: Followed the general procedure I using 2-((2-(trifluoromethyl)pyridin-4-yl)oxy)-7-azaspiro[3.5]nonane hydrochloride (120 mg, 0.418 mmol, 1.0 equiv.) and 6-chloro-1-(2,2-difluoroethyl)-1H-pyrazolo[3,4-b]pyrazine (91.6 mg, 0.418 mmol, 1.0 equiv.) as the starting materials to give 1-(2,2-difluoroethyl)-6-(2-((2-(trifluoromethyl)pyridin-4-yl)oxy)-7-azaspiro[3.5]nonan-7-yl)-1H-pyrazolo[3,4-b]pyrazine (61.1 mg, 31.1%) as a white solid. ¹H NMR (400 MHz, DMSO-d₆) δ 8.56 (d, J=5.7 Hz, 1H), 8.48 (s, 1H), 8.12 (s, 1H), 7.36 (d, J=2.4 Hz, 1H), 7.20 (dd, J=5.8, 2.4 Hz, 1H), 6.43 (tt, J=55.0, 3.9 Hz, 1H), 5.06 (p, J=6.8 Hz, 1H), 4.69 (td, J=15.0, 3.8 Hz, 2H), 3.72 (dt, J=32.0, 5.5 Hz, 4H), 2.61-2.52 (m, 2H), 1.97-1.89 (m, 2H), 1.73 (t, J=5.6 Hz, 2H), 1.67 (t, J=5.7 Hz, 2H). MS m/z: 469.2 [M+H]⁺.

### Example 704: 1-(2,2-difluoroethyl)-6-(2-((5-(trifluoromethyl)pyridin-3-yl)oxy)-7-azaspiro[3.5]nonan-7-yl)-1H-pyrazolo[3,4-b]pyrazine

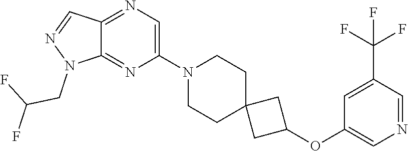 [SMILES: FC(F)Cn1ncc2ncc(N3CCC4(CC3)CC(Oc3cncc(C(F)(F)F)c3)C4)nc21]

Step 1: tert-butyl 2-((5-(trifluoromethyl)pyridin-3-yl)oxy)-7-azaspiro[3.5]nonane-7-carboxylate: Followed the general procedure E using tert-butyl 2-hydroxy-7-azaspiro[3.5]nonane-7-carboxylate (200 mg, 1.29 mmol, 1.0 equiv.) and 3-fluoro-5-(trifluoromethyl)pyridine (274 mg, 2.59 mmol, 2.0 equiv.) as the starting materials, to give tert-butyl 2-((5-(trifluoromethyl)pyridin-3-yl)oxy)-7-azaspiro[3.5]nonane-7-carboxylate (120 mg, 22%) as a white solid. MS m/z: 387 [M+H]⁺.

Step 2: 2-((5-(trifluoromethyl)pyridin-3-yl)oxy)-7-azaspiro[3.5]nonane hydrochloride: Followed the general procedure B using tert-butyl 2-((5-(trifluoromethyl)pyridin-3-yl)oxy)-7-azaspiro[3.5]nonane-7-carboxylate (120 mg) as the starting material to give the crude product 2-((5-(trifluoromethyl)pyridin-3-yl)oxy)-7-azaspiro[3.5]nonane hydrochloride (100 mg). MS m/z: 287 [M+H]⁺.

Step 3: 1-(2,2-difluoroethyl)-6-(2-((5-(trifluoromethyl)pyridin-3-yl)oxy)-7-azaspiro[3.5]nonan-7-yl)-1H-pyrazolo[3,4-b]pyrazine: Followed the general procedure I using 2-((5-(trifluoromethyl)pyridin-3-yl)oxy)-7-azaspiro[3.5]nonane hydrochloride (90 mg, 0.238 mmol, 1.0 equiv.) and 6-chloro-1-(2,2-difluoroethyl)-1H-pyrazolo[3,4-b]pyrazine (52 mg, 0.238 mmol, 1.0 equiv.) as the starting materials to give 1-(2,2-difluoroethyl)-6-(2-((5-(trifluoromethyl)pyridin-3-yl)oxy)-7-azaspiro[3.5]nonan-7-yl)-1H-pyrazolo[3,4-b]pyrazine (50 mg, 52%) as a white solid. ¹H NMR (400 MHz, DMSO-d₆) δ 8.55 (d, J=2.9 Hz, 2H), 8.48 (s, 1H), 8.12 (s, 1H), 7.65 (t, J=2.5 Hz, 1H), 6.70-6.19 (m, 1H), 5.10-4.98 (m, 1H), 4.69 (td, J=15.0, 3.8 Hz, 2H), 3.80-3.73 (m, 2H), 3.72-3.65 (m, 2H), 2.59-2.52 (m, 2H), 1.96-1.86 (m, 2H), 1.77-1.70 (m, 2H), 1.70-1.63 (m, 2H). MS m/z: 469.4 [M+H]⁺.

### Example 705: 1-(2,2-difluoroethyl)-6-(2-((4-(trifluoromethyl)pyridin-2-yl)oxy)-7-azaspiro[3.5]nonan-7-yl)-1H-pyrazolo[3,4-b]pyrazine

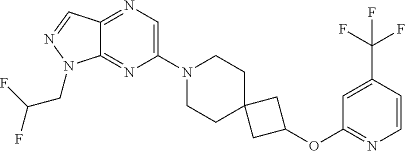 [SMILES: FC(F)Cn1ncc2ncc(N3CCC4(CC3)CC(Oc3cc(C(F)(F)F)ccn3)C4)nc21]

Step 1: tert-butyl 2-((4-(trifluoromethyl)pyridin-2-yl)oxy)-7-azaspiro[3.5]nonane-7-carboxylate: Followed the general procedure E using tert-butyl 2-hydroxy-7-azaspiro[3.5]nonane-7-carboxylate (120 mg, 0.497 mmol, 1 equiv) and 2-fluoro-4-(trifluoromethyl)pyridine (90.30 mg, 0.547 mmol, 1.1 equiv) as the starting materials to give tert-butyl 2-((4-(trifluoromethyl)pyridin-2-yl)oxy)-7-azaspiro[3.5]nonane-7-carboxylate (110 mg, 46%) as a white solid. MS m/z: 387 [M+H]⁺.

Step 2: 2-((4-(trifluoromethyl)pyridin-2-yl)oxy)-7-azaspiro[3.5]nonane hydrochloride: Followed the general procedure B using tert-butyl 2-((4-(trifluoromethyl)pyridin-2-yl)oxy)-7-azaspiro[3.5]nonane-7-carboxylate (110 mg, 0.284 mmol, 1 equiv) as the starting material to give the crude product 2-((4-(trifluoromethyl)pyridin-2-yl)oxy)-7-azaspiro[3.5]nonane hydrochloride (70 mg). MS m/z: 287 [M+H]⁺.

Step 3: 1-(2,2-difluoroethyl)-6-(2-((4-(trifluoromethyl)pyridin-2-yl)oxy)-7-azaspiro[3.5]nonan-7-yl)-1H-pyrazolo[3,4-b]pyrazine: Followed the general procedure I using 2-((4-(trifluoromethyl)pyridin-2-yl)oxy)-7-azaspiro[3.5]nonane hydrochloride (70 mg, 0.245 mmol, 1 equiv) and 6-chloro-1-(2,2-difluoroethyl)-1H-pyrazolo[3,4-b]pyrazine (64.13 mg, 0.294 mmol, 1.2 equiv) as the starting materials to give 1-(2,2-difluoroethyl)-6-(2-((4-(trifluoromethyl)pyridin-2-yl)oxy)-7-azaspiro[3.5]nonan-7-yl)-1H-pyrazolo[3,4-b]pyrazine (80 mg, 68%) as a white solid. ¹H NMR (400 MHz, DMSO-d₆) δ 8.47 (s, 1H), 8.42 (d, J=5.3 Hz, 1H), 8.12 (s, 1H), 7.32 (d, 1H), 7.19 (s, 1H), 6.43 (tt, J=54.8, 3.8 Hz, 1H), 5.34-5.23 (m, 1H), 4.69 (td, J=15.0, 3.8 Hz, 2H), 3.76 (t, J=5.6 Hz, 2H), 3.69 (t, J=5.7 Hz, 2H), 2.49-2.46 (m, 2H), 1.97-1.88 (m, 2H), 1.72 (t, J=5.7 Hz, 2H), 1.67 (t, J=5.7 Hz, 2H). MS m/z: 469.4 [M+H]⁺.

### Example 706: 1-(2,2-difluoroethyl)-6-(2-((3-(trifluoromethyl)pyridin-4-yl)oxy)-7-azaspiro[3.5]nonan-7-yl)-1H-pyrazolo[3,4-b]pyrazine

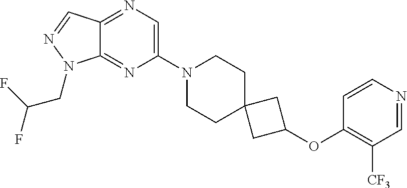 [SMILES: FC(F)Cn1ncc2ncc(N3CCC4(CC3)CC(Oc3ccncc3C(F)(F)F)C4)nc21]

Step 1: tert-butyl 2-((3-(trifluoromethyl)pyridin-4-yl)oxy)-7-azaspiro[3.5]nonane-7-carboxylate: Followed the general procedure E using tert-butyl 2-hydroxy-7-azaspiro[3.5]nonane-7-carboxylate (100 mg, 0.414 mmol, 1 equiv.) and 4-chloro-3-(trifluoromethyl)pyridine (63.3 mg, 0.414 mmol, 1.0 equiv.) as the starting materials to give tert-butyl 2-((3-(trifluoromethyl)pyridin-4-yl)oxy)-7-azaspiro[3.5]nonane-7-carboxylate (100 mg, 63%) as a white solid. MS m/z: 387 [M+H]⁺.

Step 2: 2-((3-(trifluoromethyl)pyridin-4-yl)oxy)-7-azaspiro[3.5]nonane hydrochloride: Followed the general procedure B using tert-butyl 2-((3-(trifluoromethyl)pyridin-4-yl)oxy)-7-azaspiro[3.5]nonane-7-carboxylate (100 mg) as the starting material to give the crude product 2-((3-(trifluoromethyl)pyridin-4-yl)oxy)-7-azaspiro[3.5]nonane hydrochloride (80 mg). MS m/z: 287 [M+H]⁺.

Step 3: 1-(2,2-difluoroethyl)-6-(2-((3-(trifluoromethyl)pyridin-4-yl)oxy)-7-azaspiro[3.5]nonan-7-yl)-1H-pyrazolo[3,4-b]pyrazine: Followed the general procedure I using 2-((3-(trifluoromethyl)pyridin-4-yl)oxy)-7-azaspiro[3.5]nonane hydrochloride (80 mg, 0.287 mmol, 1.0 equiv.) and 6-chloro-1-(2,2-difluoroethyl)-1H-pyrazolo[3,4-b]pyrazine (55.6 mg, 0.3 mmol, 1.0 equiv.) as the starting materials to give 1-(2,2-difluoroethyl)-6-(2-((3-(trifluoromethyl)pyridin-4-yl)oxy)-7-azaspiro[3.5]nonan-7-yl)-1H-pyrazolo[3,4-b]pyrazine (60 mg, 46%) as a white solid. ¹H NMR (400 MHz, DMSO-d₆) δ 8.72-8.65 (m, 2H), 8.48 (s, 1H), 8.12 (s, 1H), 7.18 (d, J=5.9 Hz, 1H), 6.43 (tt, J=54.8, 3.8 Hz, 1H), 5.14-5.03 (m, 1H), 4.75-4.62 (m, 2H), 3.79-3.66 (m, 4H), 2.61-2.52 (m, 2H), 1.95-1.86 (m, 2H), 1.75-1.63 (m, 4H). MS m/z: 469.1 [M+H]⁺.

### Example 707: 1-(2,2-difluoroethyl)-6-(2-((4-(trifluoromethyl)pyridin-3-yl)oxy)-7-azaspiro[3.5]nonan-7-yl)-1H-pyrazolo[3,4-b]pyrazine

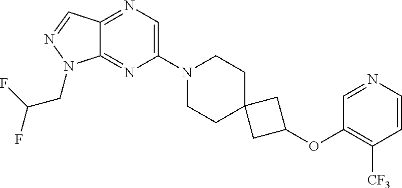 [SMILES: FC(F)Cn1ncc2ncc(N3CCC4(CC3)CC(Oc3cnccc3C(F)(F)F)C4)nc21]

Step 1: tert-butyl 2-((4-(trifluoromethyl)pyridin-3-yl)oxy)-7-azaspiro[3.5]nonane-7-carboxylate: Followed the general procedure C using tert-butyl 2-hydroxy-7-azaspiro[3.5]nonane-7-carboxylate (170 mg, 0.704 mmol, 1 equiv) and 4-(trifluoromethyl)pyridin-3-ol (172 mg, 1.05 mmol, 1.5 equiv) as the starting materials to give tert-butyl 2-((4-(trifluoromethyl)pyridin-3-yl)oxy)-7-azaspiro[3.5]nonane-7-carboxylate (100 mg, 55%) as a white solid. MS m/z: 387 [M+H]⁺.

Step 2: 2-((4-(trifluoromethylpyridin-3-yl)oxy)-7-azaspiro[3.5]nonane hydrochloride: Followed the general procedure B using tert-butyl 2-((4-(trifluoromethyl)pyridin-2-yl)oxy)-8-azaspiro[4.5]decane-8-carboxylate (100 mg, 0.348 mmol, 1 equiv) as the starting material to give the crude product 2-((4-(trifluoromethyl)pyridin-3-yl)oxy)-7-azaspiro[3.5]nonane hydrochloride (65 mg). MS m/z: 287 [M+H]⁺.

Step 3: 1-(2,2-difluoroethyl)-6-(2-((4-(trifluoromethyl)pyridin-3-yl)oxy)-7-azaspiro[3.5]nonan-7-yl)-1H-pyrazolo[3,4-b]pyrazine: Followed the general procedure I using 2-((4-(trifluoromethyl)pyridin-3-yl)oxy)-7-azaspiro[3.5]nonane hydrochloride (65 mg, 0.227 mmol, 1 equiv) and 6-chloro-1-(2,2-difluoroethyl)-1H-pyrazolo[3,4-b]pyrazine (59.55 mg, 0.272 mmol, 1.2 equiv) as the starting materials to give 1-(2,2-difluoroethyl)-6-(2-((4-(trifluoromethyl)pyridin-3-yl)oxy)-7-azaspiro[3.5]nonan-7-yl)-1H-pyrazolo[3,4-b]pyrazine (30 mg, 40%) as a white solid. ¹H NMR (400 MHz, DMSO-d₆) δ 8.55-8.46 (m, 2H), 8.41 (d, J=4.8 Hz, 1H), 8.12 (s, 1H), 7.66 (d, J=4.9 Hz, 1H), 6.43 (tt, 1H), 5.18-5.09 (m, 1H), 4.69 (td, J=15.0, 3.8 Hz, 2H), 3.72 (dt, J=24.6, 5.5 Hz, 4H), 2.60-2.54 (m, 2H), 1.95-1.86 (m, 2H), 1.75-1.64 (m, 4H). MS m/z: 469.4 [M+H]⁺.

### Example 708: 1-(2,2-difluoroethyl)-6-(2-((3-(trifluoromethyl)pyridin-2-yl)oxy)-7-azaspiro[3.5]nonan-7-yl)-1H-pyrazolo[3,4-b]pyrazine

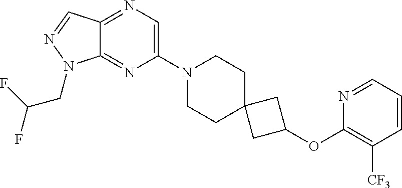 [SMILES: FC(F)Cn1ncc2ncc(N3CCC4(CC3)CC(Oc3ncccc3C(F)(F)F)C4)nc21]

Step 1: 1-(2,2-difluoroethyl)-6-(2-((3-(trifluoromethyl)pyridin-2-yl)oxy)-7-azaspiro[3.5]nonan-7-yl)-1H-pyrazolo[3,4-b]pyrazine: Followed the general procedure I using 2-((3-(trifluoromethyl)pyridin-2-yl)oxy)-7-azaspiro[3.5]nonane hydrochloride (60 mg, 0.202 mmol, 1.0 equiv.) and 6-chloro-1-(2,2-difluoroethyl)pyrazolo[3,4-b]pyrazine (44 mg, 0.202 mmol, 1 equiv.) as the starting materials to give 1-(2,2-difluoroethyl)-6-(2-((3-(trifluoromethyl)pyridin-2-yl)oxy)-7-azaspiro[3.5]nonan-7-yl)-1H-pyrazolo[3,4-b]pyrazine (80 mg, 81%) as a white solid. ¹H NMR (400 MHz, DMSO-d₆) δ 8.52-8.38 (m, 2H), 8.18-8.06 (m, 2H), 7.19-7.16 (m, 1H), 6.58-6.29 (m, 1H), 5.41-5.34 (m, 1H), 4.73-4.65 (m, 2H), 3.85-3.61 (m, 4H), 2.53-2.51 (m, 1H), 2.49 (s, 1H), 2.05-1.83 (m, 2H), 1.83-1.62 (m, 4H). MS m/z: 469.15 [M+H]⁺.

### Example 709: 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-3-one

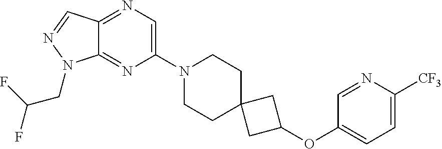 [SMILES: FC(F)Cn1ncc2ncc(N3CCC4(CC3)CC(Oc3ccc(C(F)(F)F)nc3)C4)nc21]

Step 1: tert-butyl 3-oxo-2-(6-(trifluoromethyl)pyridin-3-yl)-2,8-diazaspiro[4.5]decane-8-carboxylate: Followed the general procedure E using tert-butyl 2-hydroxy-7-azaspiro[3.5]nonane-7-carboxylate (100 mg, 0.413 mmol, 1.0 equiv.) and 5-fluoro-2-(trifluoromethyl)pyridine (82.4 mg, 0.496 mmol, 1.2 equiv.) as the starting materials to give tert-butyl 2-((6-(trifluoromethyl)pyridin-3-yl)oxy)-7-azaspiro[3.5]nonane-7-carboxylate (80 mg, 50%) as a white solid. MS m/z: 387 [M+H]⁺.

Step 2: 2-((6-(trifluoromethyl)pyridin-3-yl)oxy)-7-azaspiro[3.5]nonane hydrochloride: Followed the general procedure B using tert-butyl 2-((6-(trifluoromethyl)pyridin-3-yl)oxy)-7-azaspiro[3.5]nonane-7-carboxylate (80 mg) as the starting material to give the crude product 2-((6-(trifluoromethyl)pyridin-3-yl)oxy)-7-azaspiro[3.5]nonane hydrochloride (50 mg). MS m/z: 287 [M+H]⁺.

Step 3: 1-(2,2-difluoroethyl)-6-(2-((6-(trifluoromethyl)pyridin-3-yl)oxy)-7-azaspiro[3.5]nonan-7-yl)-1H-pyrazolo[3,4-b]pyrazine: Followed the general procedure I using 2-((6-(trifluoromethyl)pyridin-3-yl)oxy)-7-azaspiro[3.5]nonane hydrochloride (50 mg, 0.174 mmol, 1.0 equiv.) and 6-chloro-1-(2,2-difluoroethyl)-1H-pyrazolo[3,4-b]pyrazine (42 mg, 0.192 mmol, 1.1 equiv.) as the starting materials to give 1-(2,2-difluoroethyl)-6-(2-((6-(trifluoromethyl)pyridin-3-yl)oxy)-7-azaspiro[3.5]nonan-7-yl)-1H-pyrazolo[3,4-b]pyrazine (52.8 mg, 64%) as a white solid. ¹H NMR (400 MHz, DMSO-d₆) δ 8.48 (s, 1H), 8.40 (d, J=2.8 Hz, 1H), 8.12 (s, 1H), 7.83 (d, J=8.7 Hz, 1H), 7.52 (dd, J=8.5, 2.8 Hz, 1H), 6.61-6.25 (m, 1H), 5.06-4.95 (m, 1H), 4.69 (td, J=15.1, 3.8 Hz, 2H), 3.76 (t, J=5.6 Hz, 2H), 3.72-3.65 (m, 2H), 2.58-2.52 (m, 2H), 1.98-1.88 (m, 2H), 1.76-1.65 (m, 4H). MS m/z: 469.1 [M+H]⁺.

### Example 710: 1-(2,2-difluoroethyl)-6-(2-((5-(trifluoromethyl)pyridin-2-yl)oxy)-7-azaspiro[3.5]nonan-7-yl)-1H-pyrazolo[3,4-b]pyrazine

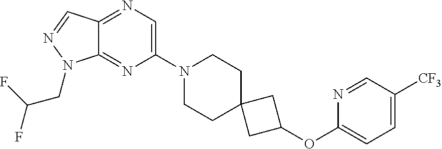 [SMILES: FC(F)Cn1ncc2ncc(N3CCC4(CC3)CC(Oc3ccc(C(F)(F)F)cn3)C4)nc21]

Step 1: tert-butyl 2-((5-(trifluoromethyl)pyridin-2-yl)oxy)-7-azaspiro[3.5]nonane-7-carboxylate: Followed the general procedure I using tert-butyl 2-hydroxy-7-azaspiro[3.5]nonane-7-carboxylate (300 mg, 1.24 mmol, 1.0 equiv.) and 2-fluoro-5-(trifluoromethyl)pyridine (246 mg, 1.492 mmol, 1.2 equiv.) as the starting materials to give tert-butyl 2-((5-(trifluoromethyl)pyridin-2-yl)oxy)-7-azaspiro[3.5]nonane-7-carboxylate (445 mg, 93%) as a yellow solid. MS m/z: 387 [M+H]⁺.

Step 2: 2-((5-(trifluoromethyl)pyridin-2-yl)oxy)-7-azaspiro[3.5]nonane hydrochloride: Followed the general procedure B using tert-butyl 2-((5-(trifluoromethyl)pyridin-2-yl)oxy)-7-azaspiro[3.5]nonane-7-carboxylate (445 mg) as the starting material to give the crude product 2-((5-(trifluoromethyl)pyridin-2-yl)oxy)-7-azaspiro[3.5]nonane hydrochloride (305 mg). MS m/z: 287 [M+H]⁺.

Step 3: 1-(2,2-difluoroethyl)-6-(2-((5-(trifluoromethyl)pyridin-2-yl)oxy)-7-azaspiro[3.5]nonan-7-yl)-1H-pyrazolo[3,4-b]pyrazine: Followed the general procedure I using 2-((5-(trifluoromethyl)pyridin-2-yl)oxy)-7-azaspiro[3.5]nonane hydrochloride (88.6 mg, 0.274 mmol, 1.0 equiv.) and 6-chloro-1-(2,2-difluoroethyl)-1H-pyrazolo[3,4-b]pyrazine (60 mg, 0.274 mmol, 1.0 equiv.) as the starting materials to give 1-(2,2-difluoroethyl)-6-(2-((5-(trifluoromethyl)pyridin-2-yl)oxy)-7-azaspiro[3.5]nonan-7-yl)-1H-pyrazolo[3,4-b]pyrazine (93.5 mg, 72%) as a white solid. ¹H NMR (400 MHz, Methanol-d₄) δ 8.46-8.41 (m, 1H), 8.34 (s, 1H), 7.98 (s, 1H), 7.92 (dd, J=8.9, 2.6 Hz, 1H), 6.92 (d, J=8.8 Hz, 1H), 6.26 (tt, J=55.4, 4.1 Hz, 1H), 5.34 (p, J=7.0 Hz, 1H), 4.67 (td, J=14.1, 4.1 Hz, 2H), 3.87-3.80 (m, 2H), 3.80-3.72 (m, 2H), 2.62-2.52 (m, 2H), 2.06-1.96 (m, 2H), 1.84-1.73 (m, 4H). MS m/z: 510.4 [M+H]⁺.

### Example 711: 1-(cyclopropylmethyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

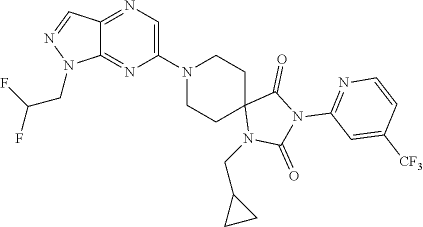 [SMILES: O=C1N(c2cc(C(F)(F)F)ccn2)C(=O)C2(CCN(c3cnc4cnn(CC(F)F)c4n3)CC2)N1CC1CC1]

Step 1: tert-butyl 4-(((benzyloxy)carbonyl)amino)-4-((4-(trifluoromethyl)pyridin-2-yl)carbamoyl)piperidine-1-carboxylate: A solution of4-(((benzyloxy)carbonyl)amino)-1-(tert-butoxycarbonyl)piperidine-4-carboxylic acid (2000 mg, 5.28 mmol, 1.0 equiv.), NMI (1300 mg, 15.8 mmol, 3.0 equiv.) and 4-(trifluoromethyl)pyridin-2-amine (1020 mg, 6.34 mmol, 1.2 equiv.) in ACN (20 mL) was stirred for overnight at 60° C. under air atmosphere. Desired product could be detected by LCMS. The residue was purified by silica gel column chromatography, eluted with PE/EtOAc (5:1) to afford tert-butyl 4-(((benzyloxy)carbonyl)amino)-4-((4-(trifluoromethyl)pyridin-2-yl)carbamoyl)piperidine-1-carboxylate (413 mg, 15%) as a white solid. MS m/z: 523 [M+H]⁺

Step 2: tert-butyl 2,4-dioxo-3-(4-(trifluoromethyl)pyridin-2-yl)-1,3,8-triazaspiro[4.5]decane-8-carboxylate: A solution of tert-butyl 4-(((benzyloxy)carbonyl)amino)-4-((4-(trifluoromethyl)pyridin-2-yl)carbamoyl)piperidine-1-carboxylate (120 mg, 0.23 mmol, 1.0 equiv.) in DMF (2 mL) was stirred for overnight at 60° C. under air atmosphere. Desired product could be detected by LCMS. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 0% to 100% gradient in 30 min; detector, UV 254 nm. The resulting mixture was concentrated under reduced pressure. This resulted in tert-butyl 2,4-dioxo-3-(4-(trifluoromethyl)pyridin-2-yl)-1,3,8-triazaspiro[4.5]decane-8-carboxylate (73 mg, 76%) as a white solid. MS m/z: 415 [M+H]⁺.

Step 3: tert-butyl 1-(cyclopropylmethyl)-2,4-dioxo-3-(4-(trifluoromethyl)pyridin-2-yl)-1,3,8-triazaspiro[4.5]decane-8-carboxylate: Followed the general procedure I using tert-butyl 2,4-dioxo-3-(4-(trifluoromethyl)pyridin-2-yl)-1,3,8-triazaspiro[4.5]decane-8-carboxylate (73 mg, 176 μmol) and (bromomethyl)cyclopropane (35.7 mg, 264 μmol, 1.5 equiv.) as the starting materials to give tert-butyl 1-(cyclopropylmethyl)-2,4-dioxo-3-(4-(trifluoromethyl)pyridin-2-yl)-1,3,8-triazaspiro[4.5]decane-8-carboxylate (60 mg, 73%) as a white solid. MS m/z: 469 [M+H]⁺.

Step 4: 1-(cyclopropylmethyl)-3-(4-(trifluoromethyl)pyridin-2-yl)-1,3,8-triazaspiro[4.5]decane-2,4-dione hydrochloride: Followed the general procedure B using tert-butyl 1-(cyclopropylmethyl)-2,4-dioxo-3-(4-(trifluoromethyl)pyridin-2-yl)-1,3,8-triazaspiro[4.5]decane-8-carboxylate (60 mg) as the starting material to give the crude product 1-(cyclopropylmethyl)-3-(4-(trifluoromethyl)pyridin-2-yl)-1,3,8-triazaspiro[4.5]decane-2,4-dione hydrochloride (50 mg). MS m/z: 369 [M+H]⁺.

Step 5: 1-(cyclopropylmethyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(cyclopropylmethyl)-3-(4-(trifluoromethyl)pyridin-2-yl)-1,3,8-triazaspiro[4.5]decane-2,4-dione hydrochloride (50 mg, 136 μmol, 1.0 equiv.) and 6-chloro-1-(2,2-difluoroethyl)-1H-pyrazolo[3,4-b]pyrazine (35.6 mg, 163 μmol, 1.2 equiv.) as the starting materials to give 1-(cyclopropylmethyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (29.7 mg, 40%) as a white solid. ¹H NMR (400 MHz, DMSO-d₆) δ 8.92 (d, J=5.2 Hz, 1H), 8.54 (s, 1H), 8.16 (s, 1H), 8.04 (s, 1H), 7.94 (dd, J=5.3, 1.7 Hz, 1H), 6.45 (tt, J=54.8, 3.8 Hz, 1H), 4.85-4.56 (m, 4H), 3.63 (t, J=12.6 Hz, 2H), 3.19 (d, J=6.8 Hz, 2H), 2.26-2.15 (m, 2H), 2.10 (d, J=13.2 Hz, 2H), 1.08 (d, J=6.9 Hz, 1H), 0.50-0.40 (m, 2H), 0.30-0.21 (m, 2H). MS m/z: 551.2 [M+H]⁺.

### Example 712: 1-(2-cyclopropylethyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

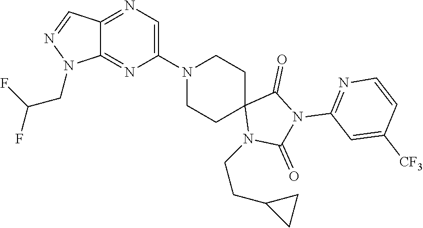 [SMILES: O=C1N(c2cc(C(F)(F)F)ccn2)C(=O)C2(CCN(c3cnc4cnn(CC(F)F)c4n3)CC2)N1CCC1CC1]

Step 1: tert-butyl 1-(2-cyclopropylethyl)-2,4-dioxo-3-(4-(trifluoromethyl)pyridin-2-yl)-1,3,8-triazaspiro[4.5]decane-8-carboxylate: A solution of tert-butyl 2,4-dioxo-3-(4-(trifluoromethyl)pyridin-2-yl)-1,3,8-triazaspiro[4.5]decane-8-carboxylate (100 mg, 0.241 mmol, 1.0 equiv.) and Cs₂CO₃ (157 mg, 0.482 mmol, 2.0 equiv.) in DMF (2 mL) was stirred for 1 h at 50° C. The resulting mixture was extracted with EtOAc (20 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (1:1) to afford tert-butyl 1-(2-cyclopropylethyl)-2,4-dioxo-3-(4-(trifluoromethyl)pyridin-2-yl)-1,3,8-triazaspiro[4.5]decane-8-carboxylate (60 mg, 52%) as a white solid. MS m/z: 483 [M+H]⁺.

Step 2: 1-(2-cyclopropylethyl)-3-(4-(trifluoromethyl)pyridin-2-yl)-1,3,8-triazaspiro[4.5]decane-2,4-dione hydrochloride: Followed the general procedure B using tert-butyl 1-(2-cyclopropylethyl)-2,4-dioxo-3-(4-(trifluoromethyl)pyridin-2-yl)-1,3,8-triazaspiro[4.5]decane-8-carboxylate (60 mg) as the starting material to give the crude product 1-(2-cyclopropylethyl)-3-(4-(trifluoromethyl)pyridin-2-yl)-1,3,8-triazaspiro[4.5]decane-2,4-dione hydrochloride (60 mg). MS m/z: 383 [M+H]⁺.

Step 3: 1-(2-cyclopropylethyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(2-cyclopropylethyl)-3-(4-(trifluoromethyl)pyridin-2-yl)-1,3,8-triazaspiro[4.5]decane-2,4-dione hydrochloride (60 mg, 0.15 mmol, 1.0 equiv.) and 6-chloro-1-(2,2-difluoroethyl)-1H-pyrazolo[3,4-b]pyrazine (38 mg, 0.17 mmol, 1.1 equiv.) as the starting materials to give 1-(2-cyclopropylethyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (50 mg, 56%) as a white solid. ¹H NMR (400 MHz, DMSO-d₆) δ 8.91 (d, J=5.1 Hz, 1H), 8.54 (s, 1H), 8.16 (s, 1H), 8.00 (s, 1H), 7.92 (dd, J=5.1, 1.7 Hz, 1H), 6.45 (tt, J=54.8, 3.8 Hz, 1H), 4.88-4.43 (m, 4H), 3.67-3.55 (m, 2H), 3.36-3.34 (m, 1H), 3.33-3.31 (m, 1H) (s, 1H), 2.25-2.04 (m, 4H), 1.51 (q, J=7.4 Hz, 2H), 0.76-0.63 (m, 1H), 0.42-0.32 (m, 2H), 0.11-0.03 (m, 2H). MS m/z: 565.4 [M+H]⁺.

### Example 713: 8-(1-(2,2-difluoroethyl)-1H-pyrazolo[3,4-b]pyrazin-6-yl)-1-(2-methoxyethyl)-3-(4-(trifluoromethyl)pyridin-2-yl)-1,3,8-triazaspiro[4.5]decane-2,4-dione

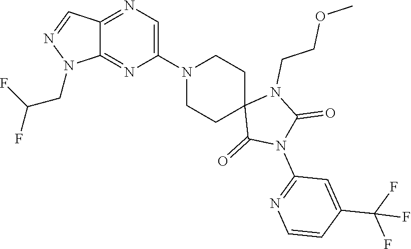 [SMILES: COCCN1C(=O)N(c2cc(C(F)(F)F)ccn2)C(=O)C12CCN(c1cnc3cnn(CC(F)F)c3n1)CC2]

Step 1: tert-butyl 1-(2-methoxyethyl)-2,4-dioxo-3-(4-(trifluoromethyl)pyridin-2-yl)-1,3,8-triazaspiro[4.5]decane-8-carboxylate: Followed the general procedure K using tert-butyl 2,4-dioxo-3-(4-(trifluoromethyl)pyridin-2-yl)-1,3,8-triazaspiro[4.5]decane-8-carboxylate (120 mg, 0.289 mmol, 1.0 equiv.) and 1-bromo-2-methoxyethane (80 mg, 0.579 mmol, 2.0 equiv.) as the starting materials to give tert-butyl 1-(2-methoxyethyl)-2,4-dioxo-3-(4-(trifluoromethyl)pyridin-2-yl)-1,3,8-triazaspiro[4.5]decane-8-carboxylate (95 mg, 69%) as a white solid. MS m/z: 473 [M+H]⁺.

Step 2: 1-(2-methoxyethyl)-3-(4-(trifluoromethyl)pyridin-2-yl)-1,3,8-triazaspiro[4.5]decane-2,4-dione hydrochloride: Followed the general procedure B using tert-butyl 1-(2-methoxyethyl)-2,4-dioxo-3-(4-(trifluoromethyl)pyridin-2-yl)-1,3,8-triazaspiro[4.5]decane-8-carboxylate (95 mg) as the starting material to give the crude product 1-(2-methoxyethyl)-3-(4-(trifluoromethyl)pyridin-2-yl)-1,3,8-triazaspiro[4.5]decane-2,4-dione hydrochloride (75 mg). MS m/z: 373 [M+H]⁺.

Step 3: 8-(1-(2,2-difluoroethyl)-1H-pyrazolo[3,4-b]pyrazin-6-yl)-1-(2-methoxyethyl)-3-(4-(trifluoromethyl)pyridin-2-yl)-1,3,8-triazaspiro[4.5]decane-2,4-dione: Followed the general procedure I using 1-(2-methoxyethyl)-3-(4-(trifluoromethyl)pyridin-2-yl)-1,3,8-triazaspiro[4.5]decane-2,4-dione hydrochloride (75 mg, 0.18 mmol, 1.0 equiv.) and 6-chloro-3-methyl-1-(oxetan-3-yl)-1H-pyrazolo[3,4-b]pyrazine (44 mg, 0.20 mmol, 1.1 equiv.) as the starting materials to give 8-(1-(2,2-difluoroethyl)-1H-pyrazolo[3,4-b]pyrazin-6-yl)-1-(2-methoxyethyl)-3-(4-(trifluoromethyl)pyridin-2-yl)-1,3,8-triazaspiro[4.5]decane-2,4-dione (28 mg, 28%) as a white solid. ¹H NMR (400 MHz, DMSO-d₆) δ 8.92 (d, J=5.1 Hz, 1H), 8.53 (s, 1H), 8.16 (s, 1H), 8.02 (d, J=1.6 Hz, 1H), 7.98-7.92 (m, 1H), 6.45 (tt, J=55.0, 3.8 Hz, 1H), 4.82-4.58 (m, 4H), 3.62 (t, J=12.6 Hz, 2H), 3.49-4.38 (m, 4H), 3.21 (s, 3H), 2.26-2.12 (m, 2H), 2.10-2.01 (m, 2H). MS m/z: 555.2 [M+H]⁺.

### Example 714: 1-(2,4-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

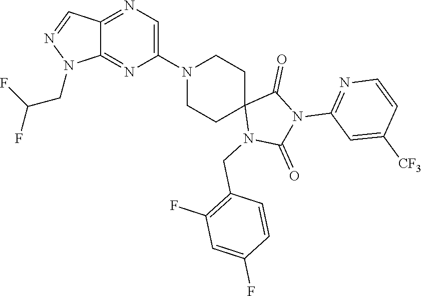 [SMILES: O=C1N(c2cc(C(F)(F)F)ccn2)C(=O)C2(CCN(c3cnc4cnn(CC(F)F)c4n3)CC2)N1Cc1ccc(F)cc1F]

Step 1: tert-butyl 1-(2,4-difluorobenzyl)-2,4-dioxo-3-(4-(trifluoromethyl)pyridin-2-yl)-1,3,8-triazaspiro[4.5]decane-8-carboxylate: Followed the general procedure K using tert-butyl 2,4-dioxo-3-(4-(trifluoromethyl)pyridin-2-yl)-1,3,8-triazaspiro[4.5]decane-8-carboxylate (50 mg, 121 μmol, 1.0 equiv.) and 1-(bromomethyl)-2,4-difluorobenzene (37.1 mg, 181 μmol, 1.5 equiv.) as the starting materials to give tert-butyl 1-(2,4-difluorobenzyl)-2,4-dioxo-3-(4-(trifluoromethyl)pyridin-2-yl)-1,3,8-triazaspiro[4.5]decane-8-carboxylate (60 mg, 92%) as a yellow oil. MS m/z: 541 [M+H]⁺.

Step 2: 1-(2,4-difluorobenzyl)-3-(4-(trifluoromethyl)pyridin-2-yl)-1,3,8-triazaspiro[4.5]decane-2,4-dione hydrochloride: Followed the general procedure B using tert-butyl 1-(2,4-difluorobenzyl)-2,4-dioxo-3-(4-(trifluoromethyl)pyridin-2-yl)-1,3,8-triazaspiro[4.5]decane-8-carboxylate (60 mg) as the starting material to give the crude product 1-(2,4-difluorobenzyl)-3-(4-(trifluoromethyl)pyridin-2-yl)-1,3,8-triazaspiro[4.5]decane-2,4-dione hydrochloride (50 mg) as a yellow solid. MS m/z: 441 [M+H]⁺.

Step 3: 1-(2,4-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(2,4-difluorobenzyl)-3-(4-(trifluoromethyl)pyridin-2-yl)-1,3,8-triazaspiro[4.5]decane-2,4-dione hydrochloride (40 mg, 90.9 μmol, 1.0 equiv.) and 6-chloro-1-(2,2-difluoroethyl)-1H-pyrazolo[3,4-b]pyrazine (23.8 mg, 109 μmol, 1.2 equiv.) as the starting materials to give 1-(2,4-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (24.1 mg, 42%) as a white solid. ¹H NMR (400 MHz, DMSO-d₆) δ 8.93 (d, J=5.1 Hz, 1H), 8.50 (s, 1H), 8.16 (s, 1H), 8.06 (s, 1H), 7.95 (d, J=5.1, 1.6 Hz, 1H), 7.54-7.45 (m, 1H), 7.22-7.14 (m, 1H), 7.00 (td, J=8.5, 2.6 Hz, 1H), 6.43 (tt, J=54.9, 3.8 Hz, 1H), 4.77-4.56 (m, 6H), 3.65-3.56 (m, 2H), 2.16-2.03 (m, 4H). MS m/z: 623.1 [M+H]⁺.

### Example 715: 1-(2,6-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

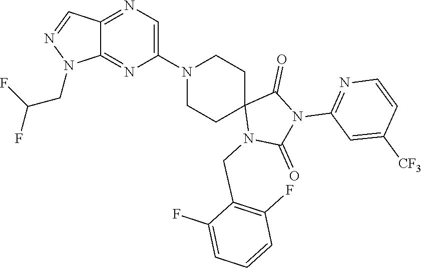 [SMILES: O=C1N(c2cc(C(F)(F)F)ccn2)C(=O)C2(CCN(c3cnc4cnn(CC(F)F)c4n3)CC2)N1Cc1c(F)cccc1F]

Step 1: tert-butyl 1-(2,6-difluorobenzyl)-2,4-dioxo-3-(4-(trifluoromethyl)pyridin-2-yl)-1,3,8-triazaspiro[4.5]decane-8-carboxylate: Followed the general procedure K using tert-butyl 2,4-dioxo-3-(4-(trifluoromethyl)pyridin-2-yl)-1,3,8-triazaspiro[4.5]decane-8-carboxylate (70 mg, 0.169 mmol, 1 equiv) and 2-(bromomethyl)-1,3-difluorobenzene (42 mg, 0.203 mmol, 1.2 equiv) as the starting materials to give tert-butyl 1-(2,6-difluorobenzyl)-2,4-dioxo-3-(4-(trifluoromethyl)pyridin-2-yl)-1,3,8-triazaspiro[4.5]decane-8-carboxylate (65 mg, 72%) as a white solid. MS m/z: 541 [M+H]⁺.

Step 2: 1-(2,6-difluorobenzyl)-3-(4-(trifluoromethyl)pyridin-2-yl)-1,3,8-triazaspiro[4.5]decane-2,4-dione hydrochloride: Followed the general procedure B using tert-butyl 1-(2,6-difluorobenzyl)-2,4-dioxo-3-(4-(trifluoromethyl)pyridin-2-yl)-1,3,8-triazaspiro[4.5]decane-8-carboxylate (65 mg, 0.12 mmol, 1 equiv) as the starting material to give the crude product 1-(2,6-difluorobenzyl)-3-(4-(trifluoromethyl)pyridin-2-yl)-1,3,8-triazaspiro[4.5]decane-2,4-dione hydrochloride (45 mg). MS m/z: 441 [M+H]⁺.

Step 3: 1-(2,6-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(2,6-difluorobenzyl)-3-(4-(trifluoromethyl)pyridin-2-yl)-1,3,8-triazaspiro[4.5]decane-2,4-dione hydrochloride (45 mg, 0.102 mmol, 1 equiv) and 6-chloro-1-(2,2-difluoroethyl)-1H-pyrazolo[3,4-b]pyrazine (26 mg, 0.122 mmol, 1.2 equiv) as the starting materials to give 1-(2,6-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (20 mg, 51%) as a white solid. ¹H NMR (400 MHz, DMSO-d₆) δ 8.91 (d, J=5.1 Hz, 1H), 8.55-8.50 (m, 1H), 8.20 (s, 1H), 8.05-7.91 (m, 2H), 7.42-7.30 (m, 1H), 6.87 (t, J=8.1 Hz, 2H), 6.45 (tt, J=54.8, 3.8 Hz, 1H), 4.80-4.53 (m, 6H), 3.66-3.55 (m, 2H), 2.16-1.95 (m, 4H). MS m/z: 623.5 [M+H]⁺.

### Example 716: 1-(3,5-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

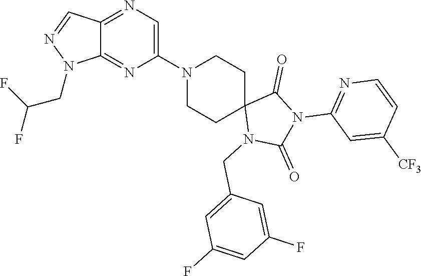 [SMILES: O=C1N(c2cc(C(F)(F)F)ccn2)C(=O)C2(CCN(c3cnc4cnn(CC(F)F)c4n3)CC2)N1Cc1cc(F)cc(F)c1]

Step 1: tert-butyl 1-(3,5-difluorobenzyl)-2,4-dioxo-3-(4-(trifluoromethyl)pyridin-2-yl)-1,3,8-triazaspiro[4.5]decane-8-carboxylate: A solution of tert-butyl 2,4-dioxo-3-(4-(trifluoromethyl)pyridin-2-yl)-1,3,8-triazaspiro[4.5]decane-8-carboxylate (80 mg, 0.193 mmol, 1.0 equiv.), 1-(bromomethyl)-3,5-difluorobenzene (60 mg, 0.289 mmol, 1.5 equiv.) and Cs₂CO₃ (125 mg, 0.386 mmol, 2.0 equiv) in DMF (1 mL) was stirred for 2 h at 50° C. The residue was purified by reversed-phase flash chromatography with the following conditions: column, C18 silica gel; mobile phase, MeCN in Water (0.1% NH₃·H₂O), 10% to 100% gradient in 30 min; detector, UV 254 nm. This resulted in tert-butyl 1-(3,5-difluorobenzyl)-2,4-dioxo-3-(4-(trifluoromethyl)pyridin-2-yl)-1,3,8-triazaspiro[4.5]decane-8-carboxylate (65 mg, 62%) as a white solid. MS m/z: 541 [M+H]⁺.

Step 2: 1-(3,5-difluorobenzyl)-3-(4-(trifluoromethyl)pyridin-2-yl)-1,3,8-triazaspiro[4.5]decane-2,4-dione hydrochloride: Followed the general procedure B using tert-butyl 1-(3,5-difluorobenzyl)-2,4-dioxo-3-(4-(trifluoromethyl)pyridin-2-yl)-1,3,8-triazaspiro[4.5]decane-8-carboxylate (50 mg) as the starting material to give the crude product 1-(3,5-difluorobenzyl)-3-(4-(trifluoromethyl)pyridin-2-yl)-1,3,8-triazaspiro[4.5]decane-2,4-dione hydrochloride (55 mg). MS m/z: 441 [M+H]⁺.

Step 3: 1-(3,5-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(3,5-difluorobenzyl)-3-(4-(trifluoromethyl)pyridin-2-yl)-1,3,8-triazaspiro[4.5]decane-2,4-dione hydrochloride (55 mg, 0.12 mmol, 1.0 equiv.) and 6-chloro-1-(2,2-difluoroethyl)-1H-pyrazolo[3,4-b]pyrazine (30 mg, 0.14 mmol, 1.1 equiv.) as the starting materials to give 1-(3,5-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (25 mg, 32%) as a white solid. ¹H NMR (400 MHz, DMSO-d₆) δ 8.94 (d, J=5.1 Hz, 1H), 8.50 (s, 1H), 8.14 (s, 1H), 8.08 (s, 1H), 7.95 (dd, J=5.2, 1.6 Hz, 1H), 7.17-7.06 (m, 3H), 6.42 (tt, J=54.9, 3.8 Hz, 1H), 4.79-4.55 (m, 6H), 3.73-3.50 (m, 2H), 2.18-2.10 (m, 2H), 2.09-2.01 (m, 2H). MS m/z: 623.4 [M+H]⁺.

### Example 717: 1-(2,3-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

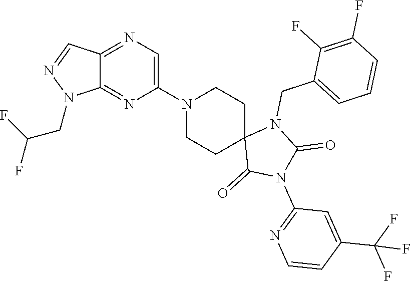 [SMILES: O=C1N(c2cc(C(F)(F)F)ccn2)C(=O)C2(CCN(c3cnc4cnn(CC(F)F)c4n3)CC2)N1Cc1cccc(F)c1F]

Step 1: tert-butyl 1-(2,3-difluorobenzyl)-2,4-dioxo-3-(4-(trifluoromethyl)pyridin-2-yl)-1,3,8-triazaspiro[4.5]decane-8-carboxylate: Followed the general procedure K using tert-butyl 2,4-dioxo-3-(4-(trifluoromethyl)pyridin-2-yl)-1,3,8-triazaspiro[4.5]decane-8-carboxylate (60 mg, 0.145 mmol) and 1-(bromomethyl)-2,3-difluorobenzene (35 mg, 0.174 mmol, 1.2 equiv.) as the starting materials to give tert-butyl 1-(2,3-difluorobenzyl)-2,4-dioxo-3-(4-(trifluoromethyl)pyridin-2-yl)-1,3,8-triazaspiro[4.5]decane-8-carboxylate (60 mg, 76%) as a white solid. MS m/z: 541 [M+H]⁺.

Step 2: 1-(2,3-difluorobenzyl)-3-(4-(trifluoromethyl)pyridin-2-yl)-1,3,8-triazaspiro[4.5]decane-2,4-dione hydrochloride: Followed the general procedure B using tert-butyl 1-(2,3-difluorobenzyl)-2,4-dioxo-3-(4-(trifluoromethyl)pyridin-2-yl)-1,3,8-triazaspiro[4.5]decane-8-carboxylate (60 mg, 0.111 mmol, 1 equiv) as the starting material to give the crude product 1-(2,3-difluorobenzyl)-3-(4-(trifluoromethyl)pyridin-2-yl)-1,3,8-triazaspiro[4.5]decane-2,4-dione hydrochloride (40 mg). MS m/z: 441 [M+H]⁺.

Step 3: 1-(2,3-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(2,3-difluorobenzyl)-3-(4-(trifluoromethyl)pyridin-2-yl)-1,3,8-triazaspiro[4.5]decane-2,4-dione hydrochloride (40 mg, 0.09 mmol, 1 equiv) and 6-chloro-1-(2,2-difluoroethyl)-1H-pyrazolo[3,4-b]pyrazine (23 mg, 0.108 mmol, 1.2 equiv) as the starting materials to give 1-(2,3-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (38 mg, 67%) as a white solid. ¹H NMR (400 MHz, DMSO-d₆) δ 8.93 (d, J=5.1 Hz, 1H), 8.50 (s, 1H), 8.15 (s, 1H), 8.06 (s, 1H), 7.95 (dd, J=5.3, 1.6 Hz, 1H), 7.39-7.21 (m, 2H), 7.18-7.08 (m, 1H), 6.43 (tt, J=54.9, 3.8 Hz, 1H), 4.80-4.58 (m, 6H), 3.67-3.55 (m, 2H), 2.20-2.03 (m, 4H). MS m/z: 623.10 [M+H]⁺.

### Example 718: 1-(2,5-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

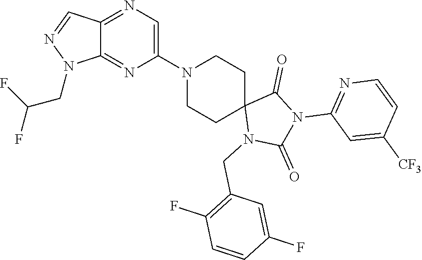 [SMILES: O=C1N(c2cc(C(F)(F)F)ccn2)C(=O)C2(CCN(c3cnc4cnn(CC(F)F)c4n3)CC2)N1Cc1cc(F)ccc1F]

Step 1: tert-butyl 1-(2,5-difluorobenzyl)-2,4-dioxo-3-(4-(trifluoromethyl)pyridin-2-yl)-1,3,8-triazaspiro[4.5]decane-8-carboxylate: Followed the general procedure I using tert-butyl 2,4-dioxo-3-(4-(trifluoromethyl)pyridin-2-yl)-1,3,8-triazaspiro[4.5]decane-8-carboxylate (70 mg, 0.169 mmol, 1 equiv.) and 2-(bromomethyl)-1,4-difluorobenzene (34.8 mg, 0.169 mmol, 1.0 equiv.) as the starting materials to give tert-butyl 1-(2,5-difluorobenzyl)-2,4-dioxo-3-(4-(trifluoromethyl)pyridin-2-yl)-1,3,8-triazaspiro[4.5]decane-8-carboxylate (50 mg, 63%) as a white solid. MS m/z: 541 [M+H]⁺.

Step 2: 1-(2,5-difluorobenzyl)-3-(4-(trifluoromethyl)pyridin-2-yl)-1,3,8-triazaspiro[4.5]decane-2,4-dione hydrochloride: Followed the general procedure B using tert-butyl 1-(2,5-difluorobenzyl)-2,4-dioxo-3-(4-(trifluoromethyl)pyridin-2-yl)-1,3,8-triazaspiro[4.5]decane-8-carboxylate (50 mg) as the starting material to give the crude product 1-(2,5-difluorobenzyl)-3-(4-(trifluoromethyl)pyridin-2-yl)-1,3,8-triazaspiro[4.5]decane-2,4-dione hydrochloride (40 mg). MS m/z: 441 [M+H]⁺.

Step 3: 1-(2,5-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(2,5-difluorobenzyl)-3-(4-(trifluoromethyl)pyridin-2-yl)-1,3,8-triazaspiro[4.5]decane-2,4-dione hydrochloride (40 mg, 0.091 mmol, 1 equiv.) and 6-chloro-1-(2,2-difluoroethyl)-1H-pyrazolo[3,4-b]pyrazine (20 mg, 0.091 mmol, 1 equiv.) as the starting materials to give 1-(2,5-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (31.4 mg, 56%) as a white solid. ¹H NMR (400 MHz, DMSO-d₆) δ 8.93 (d, J=5.1 Hz, 1H), 8.49 (s, 1H), 8.14 (s, 1H), 8.06 (s, 1H), 7.98-7.92 (m, 1H), 7.39-7.30 (m, 1H), 7.25-7.09 (m, 2H), 6.43 (tt, J=55.0, 3.8 Hz, 1H), 4.78-4.56 (m, 6H), 3.61 (t, J=12.6 Hz, 2H), 2.26-1.98 (m, 4H). MS m/z: 623.2 [M+H]⁺.

### Example 719: 1-(3,4-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

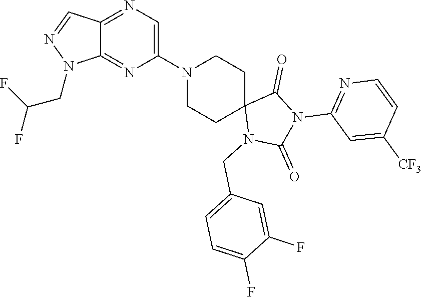 [SMILES: O=C1N(c2cc(C(F)(F)F)ccn2)C(=O)C2(CCN(c3cnc4cnn(CC(F)F)c4n3)CC2)N1Cc1ccc(F)c(F)c1]

Step 1: tert-butyl 1-(3,4-difluorobenzyl)-2,4-dioxo-3-(4-(trifluoromethyl)pyridin-2-yl)-1,3,8-triazaspiro[4.5]decane-8-carboxylate: Followed the general procedure K using tert-butyl 2,4-dioxo-3-(4-(trifluoromethyl)pyridin-2-yl)-1,3,8-triazaspiro[4.5]decane-8-carboxylate (80 mg, 193 μmol, 1.0 equiv.) and 4-(bromomethyl)-1,2-difluorobenzene (43.8 mg, 212 μmol, 1.1 equiv.) as the starting materials to give tert-butyl 1-(3,4-difluorobenzyl)-2,4-dioxo-3-(4-(trifluoromethyl)pyridin-2-yl)-1,3,8-triazaspiro[4.5]decane-8-carboxylate (60 mg, 58%) as a yellow oil. MS m/z: 541 [M+H]⁺.

Step 2: 1-(3,4-difluorobenzyl)-3-(4-trifluoromethyl)pyridin-2-yl)-1,3,8-triazaspiro[4.5]decane-2,4-dione hydrochloride: Followed the general procedure B using tert-butyl 1-(3,4-difluorobenzyl)-2,4-dioxo-3-(4-(trifluoromethyl)pyridin-2-yl)-1,3,8-triazaspiro[4.5]decane-8-carboxylate (60 mg) as the starting material to give the crude product 1-(3,4-difluorobenzyl)-3-(4-(trifluoromethyl)pyridin-2-yl)-1,3,8-triazaspiro[4.5]decane-2,4-dione hydrochloride (40 mg) as a yellow solid. MS m/z: 441 [M+H]⁺.

Step 3: 1-(3,4-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione: Followed the general procedure I using 1-(3,4-difluorobenzyl)-3-(4-(trifluoromethyl)pyridin-2-yl)-1,3,8-triazaspiro[4.5]decane-2,4-dione hydrochloride (40 mg, 90.9 μmol, 1.0 equiv.) and 6-chloro-1-(2,2-difluoroethyl)-1H-pyrazolo[3,4-b]pyrazine (23.8 mg, 109 μmol, 1.2 equiv.) as the starting materials to give 1-(3,4-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione (24.0 mg, 42%) as a white solid. ¹H NMR (400 MHz, DMSO-d₆) δ 8.93 (d, J=5.1 Hz, 1H), 8.49 (s, 1H), 8.14 (s, 1H), 8.08 (s, 1H), 7.95 (d, 1H), 7.48-7.34 (m, 2H), 7.24-7.18 (m, 1H), 6.42 (tt, J=54.9, 3.8 Hz, 1H), 4.77-4.69 (m, 2H), 4.66-4.58 (m, 4H), 3.65-3.54 (m, 2H), 2.14-2.01 (m, 4H). MS m/z: 623.1 [M+H]⁺.

### Example 720: 3-(1-(2,2-difluoroethyl)-1H-pyrazolo[3,4-b]pyrazin-6-yl)-1′-(4-(trifluoromethyl)pyridin-2-yl)-3-azaspiro[bicyclo[3.1.1]heptane-6,3′-pyrrolidin]-5′-one

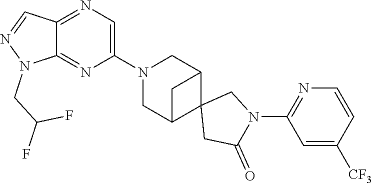 [SMILES: O=C1CC2(CN1c1cc(C(F)(F)F)ccn1)C1CC2CN(c2cnc3cnn(CC(F)F)c3n2)C1]

Step 1: benzyl 5′-oxo-1′-(4-(trifluoromethyl)pyridin-2-yl)-3-azaspiro[bicyclo[3.1.1]heptane-6,3′-pyrrolidine]-3-carboxylate: Followed the general procedure E using benzyl 5′-oxo-3-azaspiro[bicyclo[3.1.1]heptane-6,3′-pyrrolidine]-3-carboxylate (200 mg, 667 μmol, 1.0 equiv.) and 2-fluoro-4-(trifluoromethyl)pyridine (132 mg, 800 μmol, 1.2 equiv.) as the starting materials to give benzyl 5′-oxo-1′-(4-(trifluoromethyl)pyridin-2-yl)-3-azaspiro[bicyclo[3.1.1]heptane-6,3′-pyrrolidine]-3-carboxylate (80 mg, 27%) as a yellow solid. MS m/z: 446 [M+H]⁺.

Step 2: 1′-(4-(trifluoromethyl)pyridin-2-yl)-3-azaspiro[bicyclo[3.1.1]heptane-6,3′-pyrrolidin]-5′-one: Followed the general procedure U using benzyl 5′-oxo-1′-(4-(trifluoromethyl)pyridin-2-yl)-3-azaspiro[bicyclo[3.1.1]heptane-6,3′-pyrrolidine]-3-carboxylate (80 mg, 179 μmol) as the starting material to give 1′-(4-(trifluoromethyl)pyridin-2-yl)-3-azaspiro[bicyclo[3.1.1]heptane-6,3′-pyrrolidin]-5′-one (50 mg, 89%) as a yellow oil. MS m/z: 312 [M+H]⁺.

Step 3: 3-(1-(2,2-difluoroethyl)-1H-pyrazolo[3,4-b]pyrazin-6-yl)-1′-(4-(trifluoromethyl)pyridin-2-yl)-3-azaspiro[bicyclo[3.1.1]heptane-6,3′-pyrrolidin]-5′-one: Followed the general procedure I using 1′-(4-(trifluoromethyl)pyridin-2-yl)-3-azaspiro[bicyclo[3.1.1]heptane-6,3′-pyrrolidin]-5′-one (50 mg, 161 μmol, 1.0 equiv.) and 6-chloro-1-(2,2-difluoroethyl)-1H-pyrazolo[3,4-b]pyrazine (35 mg, 161 μmol, 1.0 equiv.) as the starting materials to give 3-(1-(2,2-difluoroethyl)-1H-pyrazolo[3,4-b]pyrazin-6-yl)-1′-(4-(trifluoromethyl)pyridin-2-yl)-3-azaspiro[bicyclo[3.1.1]heptane-6,3′-pyrrolidin]-5′-one (22 mg, 80%) as a white solid. ¹H NMR (400 MHz, Methanol-d₄) δ 8.68 (s, 1H), 8.63 (d, J=5.2 Hz, 1H), 8.25 (s, 1H), 8.06 (s, 1H), 7.40 (d, J=5.1 Hz, 1H), 6.30 (tt, 1H), 4.72 (td, 2H), 4.46 (s, 2H), 3.97 (s, 4H), 2.66 (s, 4H), 2.59 (s, 1H), 1.64 (d, 1H). MS m/z: 535.15 [M+H]⁺.

### Example 721: 3-(1-(2,2-difluoroethyl)-1H-pyrazolo[3,4-b]pyrazin-6-yl)-1′-(2-(trifluoromethyl)pyridin-4-yl)-3-azaspiro[bicyclo[3.1.1]heptane-6,3′-pyrrolidin]-5′-one

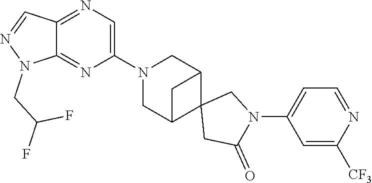 [SMILES: O=C1CC2(CN1c1ccnc(C(F)(F)F)c1)C1CC2CN(c2cnc3cnn(CC(F)F)c3n2)C1]

Step 1: benzyl 5′-oxo-1′-(2-(trifluoromethyl)pyridin-4-yl)-3-azaspiro[bicyclo[3.1.1]heptane-6,3′-pyrrolidine]-3-carboxylate: Followed the general procedure X using benzyl 5′-oxo-3-azaspiro[bicyclo[3.1.1]heptane-6,3′-pyrrolidine]-3-carboxylate (100 mg, 0.33 mmol, 1.0 equiv.) and 4-chloro-2-(trifluoromethyl)pyridine (72.4 mg, 0.36 mmol, 1.2 equiv.) as the starting materials, Xphos Pd G3 (28.2 mg, 0.03 mmol, 0.1 equiv.) as the catalyst to give benzyl 5′-oxo-1′-(2-(trifluoromethyl)pyridin-4-yl)-3-azaspiro[bicyclo[3.1.1]heptane-6,3′-pyrrolidine]-3-carboxylate (50 mg, 34%) as a yellow solid. MS m/z: 446 [M+H]⁺.

Step 2: 1′-(2-(trifluoromethyl)pyridin-4-yl)-3-azaspiro[bicyclo[3.1.1]heptane-6,3′-pyrrolidin]-5′-one: Followed the general procedure U using benzyl 5′-oxo-1′-(2-(trifluoromethyl)pyridin-4-yl)-3-azaspiro[bicyclo[3.1.1]heptane-6,3′-pyrrolidine]-3-carboxylate (50 mg, 0.11 mmol) as the starting material to give 1′-(2-(trifluoromethyl)pyridin-4-yl)-3-azaspiro[bicyclo[3.1.1]heptane-6,3′-pyrrolidin]-5′-one (25 mg, 70%) as a yellow oil. MS m/z: 312 [M+H]⁺.

Step 3: 3-(1-(2,2-difluoroethyl)-1H-pyrazolo[3,4-b]pyrazin-6-yl)-1′-(2-(trifluoromethyl)pyridin-4-yl)-3-azaspiro[bicyclo[3.1.1]heptane-6,3′-pyrrolidin]-5′-one: Followed the general procedure I using 1′-(2-(trifluoromethyl)pyridin-4-yl)-3-azaspiro[bicyclo[3.1.1]heptane-6,3′-pyrrolidin]-5′-one (25 mg, 0.08 mmol, 1.0 equiv.) and 6-chloro-1-(2,2-difluoroethyl)-1H-pyrazolo[3,4-b]pyrazine (21 mg, 0.1 mmol, 1.2 equiv.) as the starting materials to give 3-(1-(2,2-difluoroethyl)-1H-pyrazolo[3,4-b]pyrazin-6-yl)-1′-(2-(trifluoromethyl)pyridin-4-yl)-3-azaspiro[bicyclo[3.1.1]heptane-6,3′-pyrrolidin]-5′-one (18.7 mg, 47%) as a white solid. ¹H NMR (400 MHz, DMSO-d₆) δ 8.71 (d, J=5.7 Hz, 1H), 8.31-8.24 (m, 2H), 8.18 (s, 1H), 7.97-7.91 (m, 1H), 6.48 (td, J=55.0, 4.4 Hz, 1H), 4.72 (td, J=14.9, 3.8 Hz, 2H), 4.26 (s, 2H), 3.87 (br, 4H), 2.65-2.56 (m, 5H), 1.53 (d, J=9.9 Hz, 1H). MS m/z: 494.2 [M+H]⁺.

### Example 722: 3-(1-(2,2-difluoroethyl)-1H-pyrazolo[3,4-b]pyrazin-6-yl)-1′-(4-fluoro-3-(trifluoromethyl)phenyl)-3-azaspiro[bicyclo[3.1.1]heptane-6,3′-pyrrolidin]-5′-one

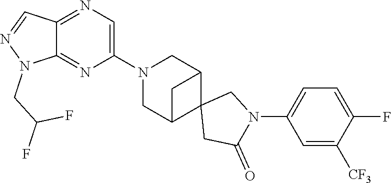 [SMILES: O=C1CC2(CN1c1ccc(F)c(C(F)(F)F)c1)C1CC2CN(c2cnc3cnn(CC(F)F)c3n2)C1]

Step 1: benzyl 6-(2-ethoxy-2-oxoethylidene)-3-azabicyclo[3.1.1]heptane-3-carboxylate: Followed the general procedure Z using benzyl 6-oxo-3-azabicyclo[3.1.1]heptane-3-carboxylate (1 g, 4.08 mmol, 1.0 equiv.) and ethyl 2-(diethoxyphosphoryl)acetate (1095 mg, 4.88 μmol, 1.1 equiv.) as the starting materials to give benzyl 6-(2-ethoxy-2-oxoethylidene)-3-azabicyclo[3.1.1]heptane-3-carboxylate (600 mg, 47%) as a yellow oil. MS m/z: 316 [M+H]⁺.

Step 2: benzyl 6-(2-ethoxy-2-oxoethyl)-6-(nitromethyl)-3-azabicyclo[3.1.1]heptane-3-carboxylate: Followed the general procedure AA using benzyl 6-(2-ethoxy-2-oxoethylidene)-3-azabicyclo[3.1.1]heptane-3-carboxylate (600 mg, 1.91 mmol) as the starting material to give benzyl 6-(2-ethoxy-2-oxoethyl)-6-(nitromethyl)-3-azabicyclo[3.1.1]heptane-3-carboxylate (510 mg, 71%) as a yellow oil. MS m/z, 377 [M+H]⁺.

Step 3: benzyl 5′-oxo-3-azaspiro[bicyclo[3.1.1]heptane-6,3′-pyrrolidine]-3-carboxylate: A solution of benzyl 6-(2-ethoxy-2-oxoethyl)-6-(nitromethyl)-3-azabicyclo[3.1.1]heptane-3-carboxylate (400 mg, 0.989 mmol, 1 equiv.) and Zn (646 mg, 9.89 mmol, 10 equiv.) and HCl (3 mL) in i-PrOH (6 mL) was stirred for overnight at 60° C. to give the crude product benzyl 5′-oxo-3-azaspiro[bicyclo[3.1.1]heptane-6,3′-pyrrolidine]-3-carboxylate (250 mg). MS m/z: 301 [M+H]⁺.

Step 4: benzyl 1′-(4-fluoro-3-(trifluoromethyl)phenyl)-5′-oxo-3-azaspiro[bicyclo[3.1.1]heptane-6,3′-pyrrolidine]-3-carboxylate: Followed the general procedure Y using benzyl 5′-oxo-3-azaspiro[bicyclo[3.1.1]heptane-6,3′-pyrrolidine]-3-carboxylate (150 mg, 0.499 mmol, 1.0 equiv.) and 4-bromo-1-fluoro-2-(trifluoromethyl)benzene (146 mg, 0.599 mmol, 1.2 equiv.) as the starting materials 1612891-29-8 (21 mg, 25 μmol, 0.05 equiv.) as the catalyst to give benzyl 1′-(4-fluoro-3-(trifluoromethyl)phenyl)-5′-oxo-3-azaspiro[bicyclo[3.1.1]heptane-6,3′-pyrrolidine]-3-carboxylate (80 mg, 35%) as a white solid. MS m/z: 463 [M+H]⁺.

Step 5: 1′-(4-fluoro-3-(trifluoromethyl)phenyl)-3-azaspiro[bicyclo[3.1.1]heptane-6,3′-pyrrolidin]-5′-one: Followed the general procedure U using benzyl 1′-(4-fluoro-3-(trifluoromethyl)phenyl)-5′-oxo-3-azaspiro[bicyclo[3.1.1]heptane-6,3′-pyrrolidine]-3-carboxylate (80 mg, 173 μmol) as the starting material to give 1′-(4-fluoro-3-(trifluoromethyl)phenyl)-3-azaspiro[bicyclo[3.1.1]heptane-6,3′-pyrrolidin]-5′-one (30 mg, 52%) as a yellow oil. MS m/z: 329 [M+H]⁺.

Step 6: 3-(1-(2,2-difluoroethyl)-1H-pyrazolo[3,4-b]pyrazin-6-yl)-1′-(4-fluoro-3-(trifluoromethyl)phenyl)-3-azaspiro[bicyclo[3.1.1]heptane-6,3′-pyrrolidin]-5′-one: Followed the general procedure I using 1′-(4-fluoro-3-(trifluoromethyl)phenyl)-3-azaspiro[bicyclo[3.1.1]heptane-6,3′-pyrrolidin]-5′-one (30 mg, 0.09 mmol, 1.0 equiv.) and 6-chloro-1-(2,2-difluoroethyl)-1H-pyrazolo[3,4-b]pyrazine (20 mg, 0.09 mmol, 1.0 equiv.) as the starting materials to give 3-(1-(2,2-difluoroethyl)-1H-pyrazolo[3,4-b]pyrazin-6-yl)-1′-(4-fluoro-3-(trifluoromethyl)phenyl)-3-azaspiro[bicyclo[3.1.1]heptane-6,3′-pyrrolidin]-5′-one (9.5 mg, 20%) as a white solid. ¹H NMR (400 MHz, Methanol-d₄) δ 8.24 (s, 1H), 8.15 (dd, J=6.4, 2.8 Hz, 1H), 8.05 (s, 1H), 7.90 (dt, J=9.0, 3.5 Hz, 1H), 7.38 (t, J=9.6 Hz, 1H), 6.30 (tt, J=55.4, 4.2 Hz, 1H), 4.72 (td, J=14.0, 4.2 Hz, 2H), 4.27 (s, 2H), 3.96 (s, 4H), 2.71-2.65 (m, 2H), 2.61-2.54 (m, 3H), 1.53 (d, J=9.9 Hz, 1H). MS m/z: 511.1 [M+H]⁺.

### Example 723: 8-(1-(2,2-difluoroethyl)-1H-pyrazolo[3,4-b]pyrazin-6-yl)-1′-(4-(trifluoromethyl)pyridin-2-yl)-8-azaspiro[bicyclo[3.2.1]octane-3,3′-pyrrolidin]-5′-one

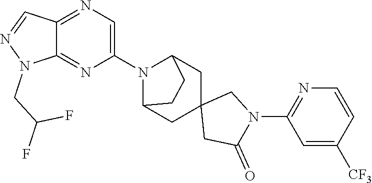 [SMILES: O=C1CC2(CC3CCC(C2)N3c2cnc3cnn(CC(F)F)c3n2)CN1c1cc(C(F)(F)F)ccn1]

Step 1: tert-butyl 3-(2-ethoxy-2-oxoethylidene)-8-azabicyclo[3.2.1]octane-8-carboxylate: Followed the general procedure Z using tert-butyl 3-oxo-8-azabicyclo[3.2.1]octane-8-carboxylate (5 g, 22.1 mmol, 1.0 equiv.) and ethyl 2-(diethoxyphosphoryl)acetate (100 mg, 26.5 mmol, 1.2 equiv.) as the starting materials to give tert-butyl 3-(2-ethoxy-2-oxoethylidene)-8-azabicyclo[3.2.1]octane-8-carboxylate (3.03 g, 46%) as a white solid. MS m/z: 296 [M+H]⁺.

Step 2: tert-butyl 3-(2-ethoxy-2-oxoethyl)-3-(nitromethyl)-8-azabicyclo[3.2.1]octane-8-carboxylate: Followed the general procedure AA using tert-butyl 3-(2-ethoxy-2-oxoethylidene)-8-azabicyclo[3.2.1]octane-8-carboxylate (3.03 g, 10.3 mmol, 1.0 equiv.) as the starting material to give tert-butyl 3-(2-ethoxy-2-oxoethyl)-3-(nitromethyl)-8-azabicyclo[3.2.1]octane-8-carboxylate (1.33 g, 36%) as a white solid. MS m/z: 357 [M+H]⁺.

Step 3: tert-butyl 5′-oxo-8-azaspiro[bicyclo[3.2.1]octane-3,3′-pyrrolidine]-8-carboxylate: Followed the general procedure AB using tert-butyl 3-(2-ethoxy-2-oxoethyl)-3-(nitromethyl)-8-azabicyclo[3.2.1]octane-8-carboxylate (400 mg, 1.12 mmol, 1.0 equiv.) as the starting material to give tert-butyl 5′-oxo-8-azaspiro[bicyclo[3.2.1]octane-3,3′-pyrrolidine]-8-carboxylate (160 mg, 50%) as a white solid. MS m/z: 281 [M+H]⁺.

Step 4: tert-butyl 3-oxo-2-(6-(trifluoromethyl)pyridin-3-yl)-2,8-diazaspiro[4.5]decane-8-carboxylate: Followed the general procedure Y using tert-butyl 5′-oxo-8-azaspiro[bicyclo[3.2.1]octane-3,3′-pyrrolidine]-8-carboxylate (160 mg, 0.569 mmol, 1.0 equiv.) and 2-bromo-4-(trifluoromethyl)pyridine (155 mg, 0.683 mmol, 1.2 equiv.) as the starting materials, Xphos Pd G3 (48.3 mg, 0.057 mmol, 0.1 equiv.) as the catalyst to give tert-butyl 5′-oxo-1′-(4-(trifluoromethyl)pyridin-2-yl)-8-azaspiro[bicyclo[3.2.1]octane-3,3′-pyrrolidine]-8-carboxylate (110 mg, 45%) as a white solid. MS m/z: 426 [M+H]⁺.

Step 5: 1′-(4-(trifluoromethyl)pyridin-2-yl)-8-azaspiro[bicyclo[3.2.1]octane-3,3′-pyrrolidin]-5′-one hydrochloride: Followed the general procedure B using tert-butyl 5′-oxo-1′-(4-(trifluoromethyl)pyridin-2-yl)-8-azaspiro[bicyclo[3.2.1]octane-3,3′-pyrrolidine]-8-carboxylate (110 mg) as the starting material to give the crude product 1′-(4-(trifluoromethyl)pyridin-2-yl)-8-azaspiro[bicyclo[3.2.1]octane-3,3′-pyrrolidin]-5′-one hydrochloride (70 mg). MS m/z: 32[MH].

Step 6: 8-(1-(2,2-difluoroethyl)-1H-pyrazolo[3,4-b]pyrazin-6-yl)-1′-(4-(trifluoromethyl)pyridin-2-yl)-8-azaspiro[bicyclo[3.2.1]octane-3,3′-pyrrolidin]-5′-one: Followed the general procedure I using 1′-(4-(trifluoromethyl)pyridin-2-yl)-8-azaspiro[bicyclo[3.2.1]octane-3,3′-pyrrolidin]-5′-one hydrochloride (70 mg, 0.215 mmol, 1.0 equiv.) and 6-chloro-1-(2,2-difluoroethyl)-1H-pyrazolo[3,4-b]pyrazine (51.7 mg, 0.236 mmol, 1.1 equiv.) as the starting materials to give 8-(1-(2,2-difluoroethyl)-1H-pyrazolo[3,4-b]pyrazin-6-yl)-1′-(4-(trifluoromethyl)pyridin-2-yl)-8-azaspiro[bicyclo[3.2.1]octane-3,3′-pyrrolidin]-5′-one (47.9 mg, 44%) as a white solid. ¹H NMR (400 MHz, DMSO-d₆) δ 8.59-8.52 (m, 2H), 8.32 (s, 1H), 8.13 (s, 1H), 7.46 (dd, J=5.1, 1.6 Hz, 1H), 6.43 (tt, J=54.8, 3.8 Hz, 1H), 4.83 (s, 2H), 4.68 (td, J=14.9, 3.8 Hz, 2H), 3.63 (s, 2H), 3.02 (s, 2H), 2.07-1.92 (m, 6H), 1.87 (d, J=13.8 Hz, 2H). MS m/z: 508.05 [M+H]⁺.

### Example 724: 3-(1-(2,2-difluoroethyl)-1H-pyrazolo[3,4-b]pyrazin-6-yl)-1′-(4-(trifluoromethyl)pyridin-2-yl)-3-azaspiro[bicyclo[3.2.1]octane-8,3′-pyrrolidin]-5′-one

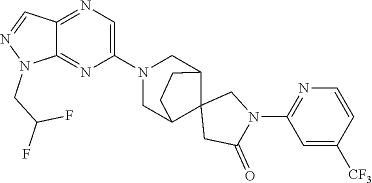 [SMILES: O=C1CC2(CN1c1cc(C(F)(F)F)ccn1)C1CCC2CN(c2cnc3cnn(CC(F)F)c3n2)C1]

Step 1: tert-butyl 8-(2-ethoxy-2-oxoethylidene)-3-azabicyclo[3.2.1]octane-3-carboxylate: Followed the general procedure Z using tert-butyl (1R,5S)-8-oxo-3-azabicyclo[3.2.1]octane-3-carboxylate (1.00 g, 4.44 mmol, 1.0 equiv.) and ethyl 2-(diethoxyphosphoryl)acetate (1095 mg, 4.88 mmol, 1.1 equiv.) as the starting materials to give tert-butyl 8-(2-ethoxy-2-oxoethylidene)-3-azabicyclo[3.2.1]octane-3-carboxylate (600 mg, 47%) as a yellow oil. MS m/z: 296 [M+H]⁺.

Step 2: tert-butyl 8-(2-ethoxy-2-oxoethyl)-8-(nitromethyl)-3-azabicyclo[3.2.1]octane-3-carboxylate: Followed the general procedure AA using tert-butyl 8-(2-ethoxy-2-oxoethylidene)-3-azabicyclo[3.2.1]octane-3-carboxylate (600 mg, 1.91 mmol) as the starting material to give tert-butyl 8-(2-ethoxy-2-oxoethyl)-8-(nitromethyl)-3-azabicyclo[3.2.1]octane-3-carboxylate (510 mg, 71%) as a yellow oil. MS m/z: 357 [M+H]⁺.

Step 3: tert-butyl 5′-oxo-3-azaspiro[bicyclo[3.2.1]octane-8,3′-pyrrolidine]-3-carboxylate: A solution of tert-butyl 8-(2-ethoxy-2-oxoethyl)-8-(nitromethyl)-3-azabicyclo[3.2.1]octane-3-carboxylate (500 mg, 1.41 mmol, 1.0 equiv.) and Zn (913 mg, 14.1 mmol, 10 equiv.) and HCl (3 mL) in i-PrOH (6 mL) was stirred for overnight at 60° C. to give the crude product tert-butyl 5′-oxo-3-azaspiro[bicyclo[3.2.1]octane-8,3′-pyrrolidine]-3-carboxylate (100 mg). MS m/z: 281 [M+H]⁺.

Step 4: tert-butyl 5′-oxo-2-(6-(trifluoromethyl)pyridin-2-yl)-3-azaspiro[bicyclo[3.2.1]octane-8,3′-pyrrolidine]-3-carboxylate: Followed the general procedure E using tert-butyl 5′-oxo-3-azaspiro[bicyclo[3.2.1]octane-8,3′-pyrrolidine]-3-carboxylate (100 mg, 0.357 mmol, 1.0 equiv.) and 2-fluoro-4-(trifluoromethyl)pyridine (71 mg, 0.428 mmol, 1.2 equiv.) as the starting materials to give tert-butyl 5′-oxo-1′-(4-(trifluoromethyl)pyridin-2-yl)-3-azaspiro[bicyclo[3.2.1]octane-8,3′-pyrrolidine]-3-carboxylate (50 mg, 33%) as a white solid. MS m/z: 426 [M+H]⁺.

Step 5: 1′-(4-(trifluoromethyl)pyridin-2-yl)-3-azaspiro[bicyclo[3.2.1]octane-8,3′-pyrrolidin]-5′-one hydrochloride: Followed the general procedure B using tert-butyl 5′-oxo-1′-(4-(trifluoromethyl)pyridin-2-yl)-3-azaspiro[bicyclo[3.2.1]octane-8,3′-pyrrolidine]-3-carboxylate (50 mg) as the starting material to give the crude product 1′-(4-(trifluoromethyl)pyridin-2-yl)-3-azaspiro[bicyclo[3.2.1]octane-8,3′-pyrrolidin]-5′-one hydrochloride (40 mg). MS m/z: 326 [M+H]⁺.

Step 6: 3-(1-(2,2-difluoroethyl)-1H-pyrazolo[3,4-b]pyrazin-6-yl)-1′-(4-(trifluoromethyl)pyridin-2-yl)-3-azaspiro[bicyclo[3.2.1]octane-8,3′-pyrrolidin]-5′-one: Followed the general procedure I using 1′-(4-(trifluoromethyl)pyridin-2-yl)-3-azaspiro[bicyclo[3.2.1]octane-8,3′-pyrrolidin]-5′-one hydrochloride (40 mg, 0.11 mmol, 1.0 equiv.) and 6-chloro-1-(2,2-difluoroethyl)-1H-pyrazolo[3,4-b]pyrazine (24.2 mg, 0.11 mmol, 1.0 equiv.) as the starting materials to give 3-(1-(2,2-difluoroethyl)-1H-pyrazolo[3,4-b]pyrazin-6-yl)-1′-(4-(trifluoromethyl)pyridin-2-yl)-3-azaspiro[bicyclo[3.2.1]octane-8,3′-pyrrolidin]-5′-one (20 mg, 35%) as a white solid. ¹H NMR (400 MHz, DMSO-d₆) δ 8.71-8.64 (m, 2H), 8.39 (s, 1H), 8.15 (s, 1H), 7.54 (dd, J=5.4, 1.7 Hz, 1H), 6.45 (tt, J=54.9, 3.8 Hz, 1H), 4.70 (td, J=14.9, 3.9 Hz, 2H), 4.13 (d, J=12.9 Hz, 2H), 3.86 (s, 2H), 3.34-3.24 (m, 2H), 2.98 (s, 2H), 2.26 (s, 2H), 1.95-1.88 (m, 2H), 1.61 (d, J=8.3 Hz, 2H). MS m/z: 508.1 [M+H]⁺.

### Example 725: 8-(1-(2,2-difluoroethyl)-1H-pyrazolo[3,4-b]pyrazin-6-yl)-6-methyl-2-(4-(trifluoromethyl)pyridin-2-yl)-2,8-diazaspiro[4.5]decan-3-one

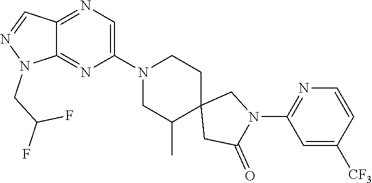 [SMILES: CC1CN(c2cnc3cnn(CC(F)F)c3n2)CCC12CC(=O)N(c1cc(C(F)(F)F)ccn1)C2]

Step 1: tert-butyl 4-(2-ethoxy-2-oxoethylidene)-3-methylpiperidine-1-carboxylate: Followed the general procedure Z using tert-butyl 3-methyl-4-oxopiperidine-1-carboxylate (2000 mg, 9.38 mmol, 1.0 equiv.) and ethyl 2-(diethoxyphosphoryl)acetate (2310 mg, 10.3 mmol, 1.1 equiv.) as the starting materials to give tert-butyl 4-(2-ethoxy-2-oxoethylidene)-3-methylpiperidine-1-carboxylate (1850 mg, 69.6%) as a white solid. MS m/z: 284 [M+H]⁺.

Step 2: tert-butyl 4-(2-ethoxy-2-oxoethyl)-3-methyl-4-(nitromethyl)piperidine-1-carboxylate: Followed the general procedure AA using tert-butyl 4-(2-ethoxy-2-oxoethylidene)-3-methylpiperidine-1-carboxylate (1850 mg, 6.51 mmol, 1.0 equiv.) as the starting material to give tert-butyl 4-(2-ethoxy-2-oxoethyl)-3-methyl-4-(nitromethyl)piperidine-1-carboxylate (300 mg, 13.3%) as a light yellow solid. MS m/z: 345 [M+H]⁺.

Step 3: tert-butyl 6-methyl-3-oxo-2,8-diazaspiro[4.5]decane-8-carboxylate: A mixture of tert-butyl 4-(2-ethoxy-2-oxoethyl)-3-methyl-4-(nitromethyl)piperidine-1-carboxylate (300 mg, 0.870 mmol, 1.0 equiv) and Zn (284 mg, 8.70 mmol, 10.0 equiv.) in 2-Propanol (7 mL) and HCl (6 M, 4 mL) was stirred for overnight at 80° C. under nitrogen atmosphere. The resulting mixture was extracted with EtOAc (3×20 mL). The combined organic layers were washed with water (2×10 mL), dried over anhydrous Na₂SO₄. After filtration, the filtrate was concentrated under reduced pressure. The residue was purified by silica gel column chromatography, eluted with EtOAc/PE=1/1 to afford tert-butyl 6-methyl-3-oxo-2,8-diazaspiro[4.5]decane-8-carboxylate (150 mg, 64.1%) as a white solid. MS m/z: 269 [M+H]⁺.

Step 4: tert-butyl 6-methyl-3-oxo-2-(4-(trifluoromethyl)pyridin-2-yl)-2,8-diazaspiro[4.5]decane-8-carboxylate: Followed the general procedure E using tert-butyl 6-methyl-3-oxo-2,8-diazaspiro[4.5]decane-8-carboxylate (150 mg, 0.558, 1.0 equiv.) and 2-fluoro-4-(trifluoromethyl)pyridine (120 mg, 0.725, 1.3 equiv.) as the starting material to give product tert-butyl 6-methyl-3-oxo-2-(4-(trifluoromethyl)pyridin-2-yl)-2,8-diazaspiro[4.5]decane-8-carboxylate (70.0 mg, 30.3%). MS m/z: 414 [M+H]⁺.

Step 5: 6-methyl-2-(4-(trifluoromethyl)pyridin-2-yl)-2,8-diazaspiro[4.5]decan-3-one hydrochloride: Followed the general procedure B using tert-butyl 6-methyl-3-oxo-2-(4-(trifluoromethyl)pyridin-2-yl)-2,8-diazaspiro[4.5]decane-8-carboxylate (70.0 mg, 0.169 mmol, 1.0 equiv.) as the starting material to give the crude product 6-methyl-2-(4-(trifluoromethyl)pyridin-2-yl)-2,8-diazaspiro[4.5]decan-3-one hydrochloride (50.0 mg). MS m/z: 314 [M+H]⁺.

Step 6: 8-(1-(2,2-difluoroethyl)-1H-pyrazolo[3,4-b]pyrazin-6-yl)-6-methyl-2-(4-(trifluoromethyl)pyridin-2-yl)-2,8-diazaspiro[4.5]decan-3-one: Followed the general procedure I using 6-methyl-2-(4-(trifluoromethyl)pyridin-2-yl)-2,8-diazaspiro[4.5]decan-3-one hydrochloride (50.0 mg, 0.160 mmol, 1.0 equiv.) and 6-chloro-1-(2,2-difluoroethyl)-1H-pyrazolo[3,4-b]pyrazine (34.9 mg, 0.160 mmol, 1.00 equiv.) as the starting materials to give 8-(1-(2,2-difluoroethyl)-1H-pyrazolo[3,4-b]pyrazin-6-yl)-6-methyl-2-(4-(trifluoromethyl)pyridin-2-yl)-2,8-diazaspiro[4.5]decan-3-one (35.9 mg, 44.5%) as a white solid. ¹H NMR (400 MHz, DMSO-d₆) δ 8.73-8.62 (m, 2H), 8.52 (s, 1H), 8.13 (d, J=1.1 Hz, 1H), 7.53 (dd, J=5.3, 1.6 Hz, 1H), 6.44 (tt, J=54.9, 3.8 Hz, 1H), 4.70 (td, J=15.0, 3.8 Hz, 2H), 4.28 (dt, J=13.7, 4.4 Hz, 1H), 4.20-4.06 (m, 1H), 3.97 (d, J=11.5 Hz, 1H), 3.91-3.81 (m, 1H), 3.33-3.25 (m, 1H), 3.17 (dd, J=13.8, 9.9 Hz, 1H), 2.92-2.54 (m, 2H), 1.98-1.80 (m, 2H), 1.78-1.58 (m, 1H), 0.90 (dd, J=12.5, 6.8 Hz, 3H). MS m/z: 496.05 [M+H]⁺.

### Example 726: 8-(1-(2,2-difluoroethyl)-1H-pyrazolo[3,4-b]pyrazin-6-yl)-7-methyl-2-(4-(trifluoromethyl)pyridin-2-yl)-2,8-diazaspiro[4.5]decan-3-one

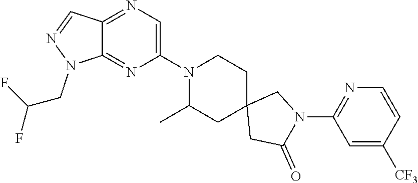 [SMILES: CC1CC2(CCN1c1cnc3cnn(CC(F)F)c3n1)CC(=O)N(c1cc(C(F)(F)F)ccn1)C2]

Step 1: tert-butyl-4-(2-ethoxy-2-oxoethylidene)-2-methylpiperidine-1-carboxylate: Followed the general procedure Z using tert-butyl 2-methyl-4-oxopiperidine-1-carboxylate (2 g, 9.39 mmol, 1.0 equiv.) and ethyl 2-(diethoxyphosphoryl)acetate (2.3 g, 10.3 mmol, 1.1 equiv.) as the starting materials to give tert-butyl-4-(2-ethoxy-2-oxoethylidene)-2-methylpiperidine-1-carboxylate (1.2 g, 45%) as a colorless oil. MS m/z: 284 [M+H]⁺.

Step 2: tert-butyl 4-(2-ethoxy-2-oxoethyl)-2-methyl-4-(nitromethyl)piperidine-1-carboxylate: Followed the general procedure AA using tert-butyl-4-(2-ethoxy-2-oxoethylidene)-2-methylpiperidine-1-carboxylate (600 mg, 2.12 mmol, 1.0 equiv.) as the starting material to give tert-butyl 4-(2-ethoxy-2-oxoethyl)-2-methyl-4-(nitromethyl)piperidine-1-carboxylate (200 mg, 27%) as a colorless oil. MS m/z: 345 [M+H]⁺.

Step 3: tert-butyl 7-methyl-3-oxo-2,8-diazaspiro[4.5]decane-8-carboxylate: To a stirred solution of tert-butyl 4-(2-ethoxy-2-oxoethyl)-2-methyl-4-(nitromethyl)piperidine-1-carboxylate (200 mg, 0.581 mmol, 1 equiv) and Zn (379 mg, 5.81 mmol, 10 equiv) in i-PrOH (2 mL) was added HCl (1 mol/L, 2 mL) at room temperature. The resulting mixture was stirred for overnight at 60° C. Desired product could be detected by LCMS. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:1) to afford tert-butyl 7-methyl-3-oxo-2,8-diazaspiro[4.5]decane-8-carboxylate (100 mg, 64%) as a colorless oil. MS m/z: 269 [M+H]⁺.

Step 4: tert-butyl 7-methyl-3-oxo-2-(4-(trifluoromethyl)pyridin-2-yl)-2,8-diazaspiro[4.5]decane-8-carboxylate: Followed the general procedure E using tert-butyl 7-methyl-3-oxo-2,8-diazaspiro[4.5]decane-8-carboxylate (100 mg, 1.66 mmol, 1.0 equiv.) and 2-fluoro-4-(trifluoromethyl)pyridine (68 mg, 1.83 mmol, 1.1 equiv.) as the starting materials to give tert-butyl 7-methyl-3-oxo-2-(4-(trifluoromethyl)pyridin-2-yl)-2,8-diazaspiro[4.5]decane-8-carboxylate (60 mg, 38%) as a colorless oil. MS m/z: [M+H]⁺.

Step 5: 7-methyl-2-(4-(trifluoromethyl)pyridin-2-yl)-2,8-diazaspiro[4.5]decan-3-one hydrochloride: Followed the general procedure B using tert-butyl 7-methyl-3-oxo-2-(4-(trifluoromethyl)pyridin-2-yl)-2,8-diazaspiro[4.5]decane-8-carboxylate (60 mg) as the starting material to give the crude product 7-methyl-2-(4-(trifluoromethyl)pyridin-2-yl)-2,8-diazaspiro[4.5]decan-3-one hydrochloride (40 mg). MS m/z: 314 [M+H]⁺.

Step 6: 8-(1-(2,2-difluoroethyl)-1H-pyrazolo[3,4-b]pyrazin-6-yl)-7-methyl-2-(4-(trifluoromethyl)pyridin-2-yl)-2,8-diazaspiro[4.5]decan-3-one: Followed the general procedure I using 7-methyl-2-(4-(trifluoromethyl)pyridin-2-yl)-2,8-diazaspiro[4.5]decan-3-one hydrochloride (40 mg, 0.114 mmol, 1.0 equiv.) and 6-chloro-1-(2,2-difluoroethyl)-1H-pyrazolo[3,4-b]pyrazine (27 mg, 0.126 mmol, 1.1 equiv.) as the starting materials to give 8-(1-(2,2-difluoroethyl)-1H-pyrazolo[3,4-b]pyrazin-6-yl)-7-methyl-2-(4-(trifluoromethyl)pyridin-2-yl)-2,8-diazaspiro[4.5]decan-3-one (8 mg, 14%) as a colorless semi-solid. ¹H NMR (400 MHz, DMSO-d₆) δ 8.65 (s, 2H), 8.44 (s, 1H), 8.13 (s, 1H), 7.52 (d, J=5.2 Hz, 1H), 6.44 (t, J=54.9 Hz, 1H), 4.93-4.87 (m, 1H), 4.79-4.62 (m, 2H), 4.44 (d, J=14.0 Hz, 1H), 3.95-3.70 (m, 2H), 3.01-2.79 (m, 2H), 2.05-1.61 (m, 4H), 1.25 (d, J=7.1 Hz, 3H). MS m/z: 496.3[M+H]⁺.

### Example 727: 8-(1-(2,2-difluoroethyl)-1H-pyrazolo[3,4-b]pyrazin-6-yl)-7,9-dimethyl-2-(4-(trifluoromethyl)pyridin-2-yl)-2,8-diazaspiro[4.5]decan-3-one

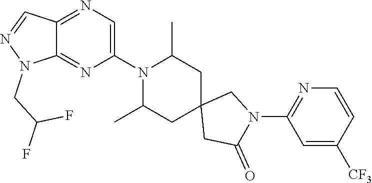 [SMILES: CC1CC2(CC(=O)N(c3cc(C(F)(F)F)ccn3)C2)CC(C)N1c1cnc2cnn(CC(F)F)c2n1]

Step 1: tert-butyl 4-(2-ethoxy-2-oxoethylidene)-2,6-dimethylpiperidine-1-carboxylate: Followed the general procedure Z using tert-butyl 2,6-dimethyl-4-oxopiperidine-1-carboxylate (2000 mg, 8.77 mmol, 1.0 equiv.) and ethyl 2-(diethoxyphosphoryl)acetate (2171 mg, 9.65 mmol, 1.1 equiv.) as the starting materials to give tert-butyl 4-(2-ethoxy-2-oxoethylidene)-2,6-dimethylpiperidine-1-carboxylate (1770 mg, 67.7%) as a white solid. MS m/z: 298 [M+H]⁺.

Step 2: tert-butyl 4-(2-ethoxy-2-oxoethyl)-2,6-dimethyl-4-(nitromethyl)piperidine-1-carboxylate: Followed the general procedure AA using tert-butyl 4-(2-ethoxy-2-oxoethylidene)-2,6-dimethylpiperidine-1-carboxylate (1770 mg, 5.94 mmol, 1.0 equiv.) as the starting material to give tert-butyl 4-(2-ethoxy-2-oxoethyl)-2,6-dimethyl-4-(nitromethyl)piperidine-1-carboxylate (600 mg, 28.1%) as a light yellow solid. MS m/z: 359 [M+H]⁺.

Step 3: tert-butyl 7,9-dimethyl-3-oxo-2,8-diazaspiro[4.5]decane-8-carboxylate: A mixture of tert-butyl 4-(2-ethoxy-2-oxoethyl)-2,6-dimethyl-4-(nitromethyl)piperidine-1-carboxylate (600 mg, 1.67 mmol, 1.0 equiv) and Zn (547 mg, 8.37 mmol, 5.0 equiv.) in 2-Propanol (36 mL) and HCl (6 M, 18 mL) was stirred for overnight at 80° C. under nitrogen atmosphere. The resulting mixture was extracted with EtOAc (3×20 mL). The combined organic layers were washed with water (2×10 mL), dried over anhydrous Na₂SO₄. After filtration, the filtrate was concentrated under reduced pressure. The residue was purified by silica gel column chromatography, eluted with EtOAc/PE=1/1 to afford tert-butyl 7,9-dimethyl-3-oxo-2,8-diazaspiro[4.5]decane-8-carboxylate (430 mg, 90.9%) as a white solid. MS m/z: 283 [M+H]⁺.

Step 4: tert-butyl 7,9-dimethyl-3-oxo-2-(4-(trifluoromethyl)pyridin-2-yl)-2,8-diazaspiro[4.5]decane-8-carboxylate: Followed the general procedure E using tert-butyl 7,9-dimethyl-3-oxo-2,8-diazaspiro[4.5]decane-8-carboxylate (200 mg, 0.707, 1.0 equiv.) and 2-fluoro-4-(trifluoromethyl)pyridine (129 mg, 0.777, 1.1 equiv.) as the starting material to give product tert-butyl 7,9-dimethyl-3-oxo-2-(4-(trifluoromethyl)pyridin-2-yl)-2,8-diazaspiro[4.5]decane-8-carboxylate (150 mg, 49.6%). MS m/z: 428 [M+H]⁺.

Step 5: 7,9-dimethyl-2-(4-(trifluoromethyl)pyridin-2-yl)-2,8-diazaspiro[4.5]decan-3-one hydrochloride: Followed the general procedure B using tert-butyl 7,9-dimethyl-3-oxo-2-(4-(trifluoromethyl)pyridin-2-yl)-2,8-diazaspiro[4.5]decane-8-carboxylate (150 mg, 0.350 mmol, 1.0 equiv.) as the starting material to give the crude product 7,9-dimethyl-2-(4-(trifluoromethyl)pyridin-2-yl)-2,8-diazaspiro[4.5]decan-3-one hydrochloride (130 mg). MS m/z: 328 [M+H]⁺.

Step 6: 8-(1-(2,2-difluoroethyl)-1H-pyrazolo[3,4-b]pyrazin-6-yl)-7,9-dimethyl-2-(4-(trifluoromethyl)pyridin-2-yl)-2,8-diazaspiro[4.5]decan-3-one: Followed the general procedure Y using 7,9-dimethyl-2-(4-(trifluoromethyl)pyridin-2-yl)-2,8-diazaspiro[4.5]decan-3-one hydrochloride (100 mg, 0.305 mmol, 1.0 equiv.) and 6-chloro-1-(2,2-difluoroethyl)-1H-pyrazolo[3,4-b]pyrazine (66.8 mg, 0.305 mmol, 1.0 equiv.) as the starting materials, Pd-PEPPSI-IPent as catalyst to give 8-(1-(2,2-difluoroethyl)-1H-pyrazolo[3,4-b]pyrazin-6-yl)-7,9-dimethyl-2-(4-(trifluoromethyl)pyridin-2-yl)-2,8-diazaspiro[4.5]decan-3-one (26.2 mg, 18.4%) as a white solid. ¹H NMR (400 MHz, DMSO-d₆) δ 8.82-8.48 (m, 2H), 8.44-8.00 (m, 2H), 7.54 (d, J=5.2 Hz, 1H), 6.43 (t, J=55.0 Hz, 1H), 4.94-4.38 (m, 4H), 4.07 (dd, J=64.4, 10.9 Hz, 2H), 3.00-2.61 (m, 2H), 2.39 (dd, J=14.0, 4.7 Hz, 1H), 2.30-2.18 (m, 1H), 2.06 (dd, J=28.9, 14.7 Hz, 2H), 1.48-1.26 (m, 6H). MS m/z: 510.25 [M+H]⁺.

### Example 728: 8-(1-(2,2-difluoroethyl)-1H-pyrazolo[3,4-b]pyrazin-6-yl)-2-(2,4-difluorophenyl)-2,8-diazaspiro[4.5]decan-3-one

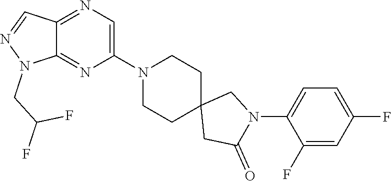 [SMILES: O=C1CC2(CCN(c3cnc4cnn(CC(F)F)c4n3)CC2)CN1c1ccc(F)cc1F]

Step 1: tert-butyl 2-(2,4-difluorophenyl)-3-oxo-2,8-diazaspiro[4.5]decane-8-carboxylate: Followed the general procedure Y using tert-butyl 3-oxo-2,8-diazaspiro[4.5]decane-8-carboxylate (200 mg, 0.786 mol, 1.0 equiv.) and 2,4-difluoro-1-iodobenzene (283 mg, 1.17 mol, 1.5 equiv.) as the starting materials, 1612891-29-8 (33 mg, 0.039 mmol, 0.05 equiv.) as the catalyst to give tert-butyl 2-(2,4-difluorophenyl)-3-oxo-2,8-diazaspiro[4.5]decane-8-carboxylate (89.5 mg, 31%) as a yellow solid. MS m/z: 367 [M+H]⁺.

Step 2: 2-(2,4-difluorophenyl)-2,8-diazaspiro[4.5]decan-3-one hydrochloride: Followed the general procedure B using tert-butyl 2-(2,4-difluorophenyl)-3-oxo-2,8-diazaspiro[4.5]decane-8-carboxylate (89.5 mg, 0.244 mmol, 1.0 equiv.) as the starting material to give the crude product 2-(2,4-difluorophenyl)-2,8-diazaspiro[4.5]decan-3-one hydrochloride (67.7 mg). MS m/z: 267 [M+H]⁺.

Step 3: 8-(1-(2,2-difluoroethyl)-1H-pyrazolo[3,4-b]pyrazin-6-yl)-2-(2,4-difluorophenyl)-2,8-diazaspiro[4.5]decan-3-one: Followed the general procedure I using 2-(2,4-difluorophenyl)-2,8-diazaspiro[4.5]decan-3-one hydrochloride (67.7 mg, 0.224 mmol, 1.0 equiv.) and 6-chloro-1-(2,2-difluoroethyl)-1H-pyrazolo[3,4-b]pyrazine (53.7 mg, 0.246 mmol, 1.1 equiv.) as the starting materials to give 8-(1-(2,2-difluoroethyl)-1H-pyrazolo[3,4-b]pyrazin-6-yl)-2-(2,4-difluorophenyl)-2,8-diazaspiro[4.5]decan-3-one (42.5 mg, 41%) as a white solid. ¹H NMR (400 MHz, DMSO-d₆) δ 8.50 (s, 1H), 8.13 (s, 1H), 7.55-7.49 (m, 1H), 7.42-7.36 (m, 1H), 7.18-7.13 (m, 1H), 6.59-6.29 (m, 1H), 4.74-4.65 (m, 2H), 3.94-3.87 (m, 2H), 3.78-3.71 (m, 2H), 3.65 (s, 2H), 2.52-2.51 (m, 2H), 1.76 (t, J=5.7 Hz, 4H). MS m/z: 449.3 [M+H]⁺.

### Example 729: 8-(1-(2,2-difluoroethyl)-1H-pyrazolo[3,4-b]pyrazin-6-yl)-2-(2-fluoro-4-(trifluoromethyl)phenyl)-2,8-diazaspiro[4.5]decan-3-one

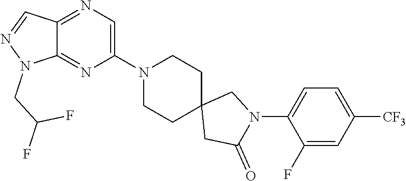 [SMILES: O=C1CC2(CCN(c3cnc4cnn(CC(F)F)c4n3)CC2)CN1c1ccc(C(F)(F)F)cc1F]

Step 1: tert-butyl 2-(2-fluoro-4-(trifluoromethyl)phenyl)-3-oxo-2,8-diazaspiro[4.5]decane-8-carboxylate: Followed the general procedure I using tert-butyl 3-oxo-2,8-diazaspiro[4.5]decane-8-carboxylate (462 mg, 1590 μmol, 1.5 equiv.) and 2-fluoro-1-iodo-4-(trifluoromethyl)benzene (462 mg, 1590 μmol, 1.5 equiv.) as the starting materials to give tert-butyl 2-(2-fluoro-4-(trifluoromethyl)phenyl)-3-oxo-2,8-diazaspiro[4.5]decane-8-carboxylate (439 mg, 45%). MS m/z: 417 [M+H]⁺.

Step 2: 2-(2-fluoro-4-(trifluoromethyl)phenyl)-2,8-diazaspiro[4.5]decan-3-one hydrochloride: Followed the general procedure B using tert-butyl 2-(2-fluoro-4-(trifluoromethyl)phenyl)-3-oxo-2,8-diazaspiro[4.5]decane-8-carboxylate (210 mg, 504 μmol, 1.0 equiv.) as the starting material to give the crude product 2-(2-fluoro-4-(trifluoromethyl)phenyl)-2,8-diazaspiro[4.5]decan-3-one hydrochloride (210 mg). MS m/z: 317 [M+H]⁺.

Step 3: 8-(1-(2,2-difluoroethyl)-1H-pyrazolo[3,4-b]pyrazin-6-yl)-2-(2-fluoro-4-(trifluoromethyl)phenyl)-2,8-diazaspiro[4.5]decan-3-one: Followed the general procedure I using 2-(2-fluoro-4-(trifluoromethyl)phenyl)-2,8-diazaspiro[4.5]decan-3-one hydrogen chloride (117 mg, 332 μmol, 1.2 equiv.) and 6-chloro-1-(2,2-difluoroethyl)-1H-pyrazolo[3,4-b]pyrazine (60 mg, 277 μmol, 1.0 equiv.) as the starting materials to give 8-(1-(2,2-difluoroethyl)-1H-pyrazolo[3,4-b]pyrazin-6-yl)-2-(2-fluoro-4-(trifluoromethyl)phenyl)-2,8-diazaspiro[4.5]decan-3-one (99.7 mg, 71%) as a white solid. ¹H NMR (400 MHz, DMSO-d₆) δ 8.50 (s, 1H), 8.13 (s, 1H), 7.84-7.64 (m, 3H), 6.44 (tt, J=54.9, 3.8 Hz, 1H), 4.70 (td, J=15.0, 3.9 Hz, 2H), 3.92 (dt, J=13.8, 5.3 Hz, 2H), 3.74 (d, J=14.1 Hz, 4H), 2.55 (s, 2H), 1.77 (t, J=5.6 Hz, 4H). MS m/z: 499.4 [M+H]⁺.

### Example 730: 8-(1-(2,2-difluoroethyl)-1H-pyrazolo[3,4-b]pyrazin-6-yl)-2-(2,4-difluorophenyl)-2,8-diazaspiro[4.5]decane

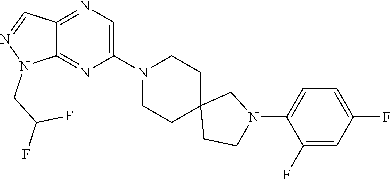 [SMILES: Fc1ccc(N2CCC3(CCN(c4cnc5cnn(CC(F)F)c5n4)CC3)C2)c(F)c1]

Step 1: tert-butyl 2-((5-(trifluoromethyl)pyridin-3-yl)oxy)-8-azaspiro[4.5]decane-8-carboxylate: Followed the general procedure C using tert-butyl 2-hydroxy-8-azaspiro[4.5]decane-8-carboxylate (250 mg, 981 μmol, 1.0 equiv.) and 5-(trifluoromethyl)pyridin-3-ol (160 mg, 981 μmol, 1.0 equiv.) as the starting materials to give tert-butyl 2-((5-(trifluoromethyl)pyridin-3-yl)oxy)-8-azaspiro[4.5]decane-8-carboxylate (354 mg, 90%). MS m/z: 353 [M+H]⁺.

Step 2: 2-((5-(trifluoromethyl)pyridin-3-yl)oxy)-8-azaspiro[4.5]decane hydrochloride: Followed the general procedure B using tert-butyl 2-((5-(trifluoromethyl)pyridin-3-yl)oxy)-8-azaspiro[4.5]decane-8-carboxylate (354 mg) as the starting material to give the crude product 2-((5-(trifluoromethyl)pyridin-3-yl)oxy)-8-azaspiro[4.5]decane hydrochloride (354 mg). MS m/z: 253 [M+H]⁺.

Step 3: 8-(1-(2,2-difluoroethyl)-1H-pyrazolo[3,4-b]pyrazin-6-yl)-2-(2,4-difluorophenyl)-2,8-diazaspiro[4.5]decane: Followed the general procedure I using 2-(2,4-difluorophenyl)-2,8-diazaspiro[4.5]decane hydrochloride (111 mg, 329 μmol, 1.2 equiv.) and 6-chloro-1-(2,2-difluoroethyl)-1H-pyrazolo[3,4-b]pyrazine (60 mg, 274 μmol, 1.0 equiv.) as the starting materials to give 8-(1-(2,2-difluoroethyl)-1H-pyrazolo[3,4-b]pyrazin-6-yl)-2-(2,4-difluorophenyl)-2,8-diazaspiro[4.5]decane (95 mg, 70%) as a white solid. ¹H NMR (400 MHz, DMSO-d₆) δ 8.48 (s, 1H), 8.13 (s, 1H), 7.16-7.05 (m, 1H), 6.96-6.86 (m, 1H), 6.82-6.70 (m, 1H), 6.61-6.28 (m, 1H), 4.69 (td, J=15.0, 3.8 Hz, 2H), 3.80 (t, J=5.6 Hz, 4H), 3.40 (td, J=7.0, 2.6 Hz, 2H), 3.21 (d, J=2.0 Hz, 2H), 1.87 (t, J=7.0 Hz, 2H), 1.74-1.59 (m, 4H). MS m/z: 435.3 [M+H]⁺.

### Example 731: 8-(1-(2,2-difluoroethyl)-1H-pyrazolo[3,4-b]pyrazin-6-yl)-2-(6-(trifluoromethyl)pyridazin-4-yl)-2,8-diazaspiro[4.5]decane

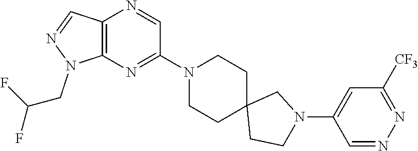 [SMILES: FC(F)Cn1ncc2ncc(N3CCC4(CCN(c5cnnc(C(F)(F)F)c5)C4)CC3)nc21]

Step 1: 8-[1-(2,2-difluoroethyl)pyrazolo[3,4-b]pyrazin-6-yl]-2-[6-(trifluoromethyl)pyridazin-4-yl]-2,8-diazaspiro[4.5]decane: Followed the general procedure I using 2-(6-(trifluoromethyl)pyridazin-4-yl)-2,8-diazaspiro[4.5]decane hydrochloride (40 mg, 0.124 mmol, 1.0 equiv.) and 6-chloro-1-(2,2-difluoroethyl)pyrazolo[3,4-b]pyrazine (27 mg, 0.124 mmol, 1.0 equiv.) as the starting materials to give 8-[1-(2,2-difluoroethyl)pyrazolo[3,4-b]pyrazin-6-yl]-2-[6-(trifluoromethyl)pyridazin-4-yl]-2,8-diazaspiro[4.5]decane (5 mg, 8%) as a white solid. ¹H NMR (400 MHz, Methanol-d₄) δ 8.74 (s, 1H), 8.36 (s, 1H), 8.00 (s, 1H), 6.99 (s, 1H), 6.26 (tt, 1H), 4.66 (td, 2H), 4.00-3.93 (m, 2H), 3.87-3.81 (m, 2H), 3.72-3.38 (m, 4H), 2.12-2.08 (m, 2H), 1.86-1.72 (m, 4H). MS m/z: 469.2 [M+H]⁺.

### Example 732: 8-(1-(2,2-difluoroethyl)-1H-pyrazolo[3,4-b]pyrazin-6-yl)-2-(6-(trifluoromethyl)pyridazin-3-yl)-2,8-diazaspiro[4.5]decane

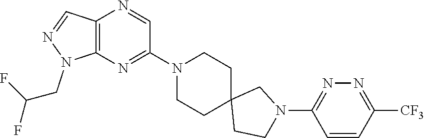 [SMILES: FC(F)Cn1ncc2ncc(N3CCC4(CCN(c5ccc(C(F)(F)F)nn5)C4)CC3)nc21]

Step 1: tert-butyl 2-(6-(trifluoromethyl)pyridazin-3-yl)-2,8-diazaspiro[4.5]decane-8-carboxylate: Followed the general procedure I using tert-butyl 2,8-diazaspiro[4.5]decane-8-carboxylate (200 mg, 0.833 mmol, 1.0 equiv.) and 3-bromo-6-(trifluoromethyl)pyridazine (207 mg, 0.916 mmol, 1.1 equiv.) as the starting materials to give tert-butyl 2-(6-(trifluoromethyl)pyridazin-3-yl)-2,8-diazaspiro[4.5]decane-8-carboxylate (180 mg, 56%) as a colorless oil. MS m/z: 387 [M+H]⁺.

Step 2: 2-(2-(2,2,2-trifluoroethoxy)pyrimidin-5-yl)-2,8-diazaspiro[4.5]decane hydrochloride: Followed the general procedure B using tert-butyl 2-(6-(trifluoromethyl)pyridazin-3-yl)-2,8-diazaspiro[4.5]decane-8-carboxylate (180 mg) as the starting material to give the crude product 2-(6-(trifluoromethyl)pyridazin-3-yl)-2,8-diazaspiro[4.5]decane hydrochloride (160 mg). MS m/z: 287 [M+H]⁺.

Step 3: 8-(1-(2,2-difluoroethyl)-1H-pyrazolo[3,4-b]pyrazin-6-yl)-2-(2-(2,2,2-trifluoroethoxy)pyrimidin-5-yl)-2,8-diazaspiro[4.5]decane: Followed the general procedure I using 2-(6-(trifluoromethyl)pyridazin-3-yl)-2,8-diazaspiro[4.5]decane hydrochloride (60 mg, 0.186 mmol, 1.0 equiv.) and 6-chloro-1-(2,2-difluoroethyl)-1H-pyrazolo[3,4-b]pyrazine (45 mg, 0.204 mol, 1.1 equiv.) as the starting materials to give 8-(1-(2,2-difluoroethyl)-1H-pyrazolo[3,4-b]pyrazin-6-yl)-2-(6-(trifluoromethyl)pyridazin-3-yl)-2,8-diazaspiro[4.5]decane (35 mg, 40%) as a white solid. ¹H NMR (300 MHz, DMSO-d₆) δ 8.57 (s, 1H), 8.21 (s, 1H), 7.85 (d, J=9.5 Hz, 1H), 7.10 (d, J=9.5 Hz, 1H), 6.52 (tt, J=54.9, 3.8 Hz, 1H), 4.77 (td, J=15.0, 3.8 Hz, 2H), 4.05-3.53 (m, 8H), 2.07 (t, J=7.0 Hz, 2H), 1.76 (d, J=8.6 Hz, 4H). MS m/z: 469.3[M+H]⁺.

### Example 733: 8-(1-(2,2-difluoroethyl)-1H-pyrazolo[3,4-b]pyrazin-6-yl)-2-(6-(trifluoromethyl)pyrazin-2-yl)-2,8-diazaspiro[4.5]decane

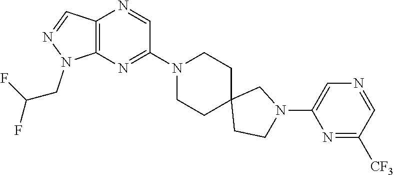 [SMILES: FC(F)Cn1ncc2ncc(N3CCC4(CCN(c5cncc(C(F)(F)F)n5)C4)CC3)nc21]

Step 1: tert-butyl 2-(6-(trifluoromethyl)pyrazin-2-yl)-2,8-diazaspiro[4.5]decane-8-carboxylate: Followed the general procedure I using tert-butyl 2,8-diazaspiro[4.5]decane-8-carboxylate (100 mg, 0.414 mmol, 1.0 equiv.) and 2-chloro-6-(trifluoromethyl)pyrazine (91.1 mg, 0.498 mmol, 1.2 equiv.) as the starting materials to give tert-butyl 2-(6-(trifluoromethyl)pyrazin-2-yl)-2,8-diazaspiro[4.5]decane-8-carboxylate (90.0 mg, 56.0%) as a light yellow oil. MS m/z: 387 [M+H]⁺.

Step 2: 2-(6-(trifluoromethyl)pyrazin-2-yl)-2,8-diazaspiro[4.5]decane hydrochloride: Followed the general procedure B using tert-butyl 2-(6-(trifluoromethyl)pyrazin-2-yl)-2,8-diazaspiro[4.5]decane-8-carboxylate (90.0 mg, 0.233 mmol, 1.0 equiv.) as the starting material to give the crude product 2-(6-(trifluoromethyl)pyrazin-2-yl)-2,8-diazaspiro[4.5]decane hydrochloride (70.0 mg). MS m/z: 287 [M+H]⁺.

Step 3: 8-(1-(2,2-difluoroethyl)-1H-pyrazolo[3,4-b]pyrazin-6-yl)-2-(6-(trifluoromethyl)pyrazin-2-yl)-2,8-diazaspiro[4.5]decane: Followed the general procedure I using 2-(6-(trifluoromethyl)pyrazin-2-yl)-2,8-diazaspiro[4.5]decane hydrochloride (70.0 mg, 0.244 mmol, 1.0 equiv.) and 6-chloro-1-(2,2-difluoroethyl)-1H-pyrazolo[3,4-b]pyrazine (53.4 mg, 0.244 mmol, 1.0 equiv.) as the starting materials to give 8-(1-(2,2-difluoroethyl)-1H-pyrazolo[3,4-b]pyrazin-6-yl)-2-(6-(trifluoromethyl)pyrazin-2-yl)-2,8-diazaspiro[4.5]decane (67.0 mg, 58.4%) as a white semi-solid. ¹H NMR (400 MHz, DMSO-d₆) δ 8.49 (s, 1H), 8.30 (s, 1H), 8.17 (s, 1H), 8.13 (s, 1H), 6.44 (tt, J=54.9, 3.8 Hz, 1H), 4.70 (td, J=15.0, 3.8 Hz, 2H), 3.82 (d, J=13.7 Hz, 4H), 3.61 (t, J=7.0 Hz, 2H), 3.47 (s, 2H), 1.98 (d, J=7.2 Hz, 2H), 1.70 (dt, J=13.0, 7.9 Hz, 4H). MS m/z: 469.4 [M+H]⁺.

### Example 734: 8-(1-(2,2-difluoroethyl)-1H-pyrazolo[3,4-b]pyrazin-6-yl)-2-(5-(trifluoromethyl)pyrazin-2-yl)-2,8-diazaspiro[4.5]decane

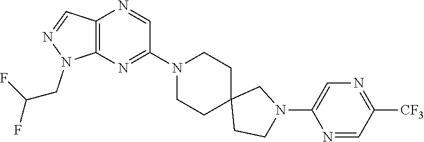 [SMILES: FC(F)Cn1ncc2ncc(N3CCC4(CCN(c5cnc(C(F)(F)F)cn5)C4)CC3)nc21]

Step 1: tert-butyl 2-(5-(trifluoromethyl)pyrazin-2-yl)-2,8-diazaspiro[4.5]decane-8-carboxylate: Followed the general procedure I using tert-butyl 2,8-diazaspiro[4.5]decane-8-carboxylate (100 mg, 0.415 mmol, 1.0 equiv.) and 2-chloro-5-(trifluoromethyl)pyrazine (91.1 mg, 0.498 mmol, 1.2 equiv.) as the starting materials to give tert-butyl 2-(5-(trifluoromethyl)pyrazin-2-yl)-2,8-diazaspiro[4.5]decane-8-carboxylate (120 mg, 74%) as a white solid. MS m/z: 387 [M+H]⁺.

Step 2: 2-(5-(trifluoromethyl)pyrazin-2-yl)-2,8-diazaspiro[4.5]decane hydrochloride: Followed the general procedure B using tert-butyl 2-(5-(trifluoromethyl)pyrazin-2-yl)-2,8-diazaspiro[4.5]decane-8-carboxylate (120 mg) as the starting material to give the crude product 2-(5-(trifluoromethyl)pyrazin-2-yl)-2,8-diazaspiro[4.5]decane hydrochloride (80 mg). MS m/z: 287 [M+H]⁺.

Step 3: 8-(1-(2,2-difluoroethyl)-1H-pyrazolo[3,4-b]pyrazin-6-yl)-2-(5-(trifluoromethyl)pyrazin-2-yl)-2,8-diazaspiro[4.5]decane: Followed the general procedure I using 2-(5-(trifluoromethyl)pyrazin-2-yl)-2,8-diazaspiro[4.5]decane hydrochloride (80 mg, 0.279 mmol, 1.0 equiv.) and 6-chloro-1-(2,2-difluoroethyl)-1H-pyrazolo[3,4-b]pyrazine (67.2 mg, 0.307 mmol, 1.1 equiv.) as the starting materials to give 8-(1-(2,2-difluoroethyl)-1H-pyrazolo[3,4-b]pyrazin-6-yl)-2-(5-(trifluoromethyl)pyrazin-2-yl)-2,8-diazaspiro[4.5]decane (81.3 mg, 62%) as a white solid. ¹H NMR (400 MHz, DMSO-d₆) δ 8.48 (d, J=11.3 Hz, 2H), 8.11 (d, J=18.5 Hz, 2H), 6.44 (tt, J=54.9, 3.8 Hz, 1H), 4.70 (td, J=15.0, 3.8 Hz, 2H), 3.95-3.84 (m, 2H), 3.81-3.70 (m, 2H), 3.64 (t, J=7.1 Hz, 2H), 3.51 (s, 2H), 1.98 (s, 2H), 1.76-1.60 (m, 4H). MS m/z: 469.2 [M+H]⁺.

### Example 735: 8-(1-(2,2-difluoroethyl)-1H-pyrazolo[3,4-b]pyrazin-6-yl)-2-(3-(trifluoromethyl)pyrazin-2-yl)-2,8-diazaspiro[4.5]decane

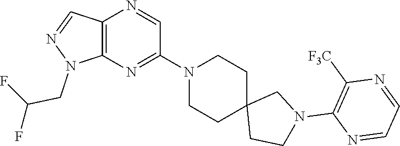 [SMILES: FC(F)Cn1ncc2ncc(N3CCC4(CC3)CCN(c3nccnc3C(F)(F)F)C4)nc21]

Step 1: tert-butyl 2-(3-(trifluoromethyl)pyrazin-2-yl)-2,8-diazaspiro[4.5]decane-8-carboxylate: Followed the general procedure I using tert-butyl 2,8-diazaspiro[4.5]decane-8-carboxylate (150 mg, 0.662 mmol, 1.0 equiv.) and 2-chloro-3-(trifluoromethyl)pyrazine (137 mg, 0.747 mmol, 1.2 equiv.) as the starting materials to give tert-butyl 2-(3-(trifluoromethyl)pyrazin-2-yl)-2,8-diazaspiro[4.5]decane-8-carboxylate (120 mg, 49.8%) as a light yellow oil. MS m/z: 387 [M+H]⁺.

Step 2: 2-(3-(trifluoromethyl)pyrazin-2-yl)-2,8-diazaspiro[4.5]decane hydrochloride: Followed the general procedure B using tert-butyl 2-(3-(trifluoromethyl)pyrazin-2-yl)-2,8-diazaspiro[4.5]decane-8-carboxylate (120 mg, 0.310 mmol, 1.0 equiv.) as the starting material to give the crude product 2-(3-(trifluoromethyl)pyrazin-2-yl)-2,8-diazaspiro[4.5]decane hydrochloride (70.0 mg). MS m/z: 287 [M+H]⁺.

Step 3: 8-(1-(2,2-difluoroethyl)-1H-pyrazolo[3,4-b]pyrazin-6-yl)-2-(3-(trifluoromethyl)pyrazin-2-yl)-2,8-diazaspiro[4.5]decane: Followed the general procedure I using 2-(3-(trifluoromethyl)pyrazin-2-yl)-2,8-diazaspiro[4.5]decane hydrochloride (90.0 mg, 0.314 mmol, 1.0 equiv.) and 6-chloro-1-(2,2-difluoroethyl)-1H-pyrazolo[3,4-b]pyrazine (53.4 mg, 0.314 mmol, 1.0 equiv.) as the starting materials to give 8-(1-(2,2-difluoroethyl)-1H-pyrazolo[3,4-b]pyrazin-6-yl)-2-(3-(trifluoromethyl)pyrazin-2-yl)-2,8-diazaspiro[4.5]decane (37.9 mg, 32.7%) as a white solid. ¹H NMR (400 MHz, DMSO-d₆) δ 8.48 (s, 1H), 8.39 (d, J=2.2 Hz, 1H), 8.13 (s, 1H), 8.02 (d, J=2.2 Hz, 1H), 6.44 (tt, J=54.9, 3.8 Hz, 1H), 4.69 (td, J=15.0, 3.8 Hz, 2H), 3.90-3.73 (m, 4H), 3.65 (t, J=7.0 Hz, 2H), 3.46 (s, 2H), 1.95 (t, J=7.0 Hz, 2H), 1.72-1.60 (m, 4H). MS m/z: 469.4 [M+H]⁺.

### Example 736: 8-(1-(2,2-difluoroethyl)-1H-pyrazolo[3,4-b]pyrazin-6-yl)-2-(2-(trifluoromethyl)pyrimidin-5-yl)-2,8-diazaspiro[4.5]decane

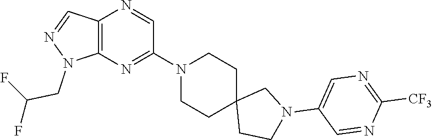 [SMILES: FC(F)Cn1ncc2ncc(N3CCC4(CCN(c5cnc(C(F)(F)F)nc5)C4)CC3)nc21]

Step 1: tert-butyl 2-(2-(trifluoromethyl)pyrimidin-5-yl)-2,8-diazaspiro[4.5]decane-8-carboxylate: Followed the general procedure I using tert-butyl 2,8-diazaspiro[4.5]decane-8-carboxylate (100 mg, 416 μmol, 1.0 equiv.) and 5-bromo-2-(trifluoromethyl)pyrimidine (103 mg, 458 μmol, 1.1 equiv.) as the starting materials to give tert-butyl 2-(2-(trifluoromethyl)pyrimidin-5-yl)-2,8-diazaspiro[4.5]decane-8-carboxylate (100 mg, 62%) as a yellow oil. MS m/z: 387 [M+H]⁺.

Step 1: 2-(2-(trifluoromethyl)pyrimidin-5-yl)-2,8-diazaspiro[4.5]decane hydrogen chloride: Followed the general procedure B using tert-butyl 2-[2-(trifluoromethyl) pyrimidin-5-yl]-2,8-diazaspiro[4.5]decane-8-carboxylate (100 mg, 259 μmol, 1.0 equiv.) as the starting material to give the crude product 2-(2-(trifluoromethyl)pyrimidin-5-yl)-2,8-diazaspiro[4.5]decane hydrochloride (100 mg). MS m/z: 287 [M+H]⁺.

Step 3: 8-(1-(2,2-difluoroethyl)-1H-pyrazolo[3,4-b]pyrazin-6-yl)-2-(2-(trifluoromethyl)pyrimidin-5-yl)-2,8-diazaspiro[4.5]decane: Followed the general procedure I using 6-chloro-1-(2,2-difluoroethyl)pyrazolo[3,4-b]pyrazine (70 mg, 320 μmol, 1.0 equiv.) and 2-(2-(trifluoromethyl)pyrimidin-5-yl)-2,8-diazaspiro[4.5]decane hydrochloride (100 mg, 352 μmol, 1.1 equiv.) as the starting materials to give 8-[1-(2,2-difluoroethyl)pyrazolo[3,4-b]pyrazin-6-yl]-2-[2-(trifluoromethyl)pyrimidin-5-yl]-2,8-diazaspiro[4.5]decane (36.7 mg, 24%) as a white solid. ¹H NMR (400 MHz, DMSO-d₆) δ 8.48 (s, 1H), 8.22 (s, 2H), 8.12 (s, 1H), 6.44 (tt, J=54.9, 3.8 Hz, 1H), 4.70 (td, J=15.0, 3.8 Hz, 2H), 3.90 (dt, J=13.4, 4.8 Hz, 2H), 3.74 (ddd, J=12.8, 7.2, 4.2 Hz, 2H), 3.52 (s, 2H), 1.98 (t, J=7.0 Hz, 2H), 1.72-1.60 (m, 4H). MS m/z: 469.25 [M+H]⁺

### Example 737: 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-3-one

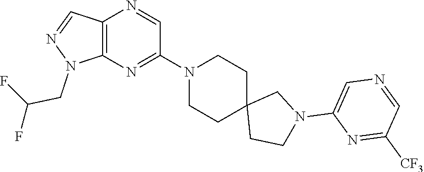 [SMILES: FC(F)Cn1ncc2ncc(N3CCC4(CCN(c5cncc(C(F)(F)F)n5)C4)CC3)nc21]

Step 1: tert-butyl 2-(2-(trifluoromethyl)pyrimidin-4-yl)-2,8-diazaspiro[4.5]decane-8-carboxylate: Followed the general procedure I using tert-butyl 2,8-diazaspiro[4.5]decane-8-carboxylate (100 mg, 0.413 mmol, 1.0 equiv.) and 4-fluoro-2-(trifluoromethyl)pyrimidine (82.4 mg, 0.496 mmol, 1.2 equiv.) as the starting materials to give tert-butyl 2-(2-(trifluoromethyl)pyrimidin-4-yl)-2,8-diazaspiro[4.5]decane-8-carboxylate (120 mg, 74%) as a white solid. MS m/z: 387 [M+H]⁺.

Step 2: 2-(2-(trifluoromethyl)pyrimidin-4-yl)-2,8-diazaspiro[4.5]decane hydrochloride: Followed the general procedure B using tert-butyl 2-(2-(trifluoromethyl)pyrimidin-4-yl)-2,8-diazaspiro[4.5]decane-8-carboxylate (120 mg) as the starting material to give the crude product 2-(2-(trifluoromethyl)pyrimidin-4-yl)-2,8-diazaspiro[4.5]decane hydrochloride (80 mg). MS m/z: 287 [M+H]⁺.

Step 3: 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decane: Followed the general procedure I using 2-((6-(trifluoromethyl)pyridin-3-yl)oxy)-7-azaspiro[3.5]nonane (80 mg, 0.279 mmol, 1.0 equiv.) and 6-chloro-1-(2,2-difluoroethyl)-1H-pyrazolo[3,4-b]pyrazine (67.2 mg, 0.307 mmol, 1.1 equiv.) as the starting materials to give 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decane (96.6 mg, 74%) as a white solid. ¹H NMR (400 MHz, DMSO-d₆) δ 8.49 (d, J=4.0 Hz, 1H), 8.30 (d, J=6.1 Hz, 1H), 8.13 (s, 1H), 6.73 (d, J=6.2 Hz, 1H), 6.61-6.28 (m, 1H), 4.75-4.63 (m, 2H), 3.92-3.71 (m, 4H), 3.64 (t, J=7.2 Hz, 1H), 3.53 (t, J=7.1 Hz, 1H), 3.48 (s, 1H), 3.39 (s, 1H), 2.01 (t, J=7.0 Hz, 1H), 1.93 (t, J=7.1 Hz, 1H), 1.68 (s, 4H). MS m/z: 469.1 [M+H]⁺.

### Example 738: 8-(1-(2,2-difluoroethyl)-1H-pyrazolo[3,4-b]pyrazin-6-yl)-2-(2-(2,2,2-trifluoroethoxy)pyrimidin-4-yl)-2,8-diazaspiro[4.5]decane

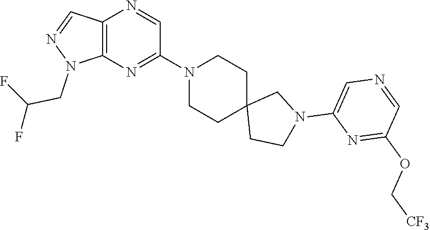 [SMILES: FC(F)Cn1ncc2ncc(N3CCC4(CCN(c5cncc(OCC(F)(F)F)n5)C4)CC3)nc21]

Step 1: tert-butyl 2-(2-chloropyrimidin-4-yl)-2,8-diazaspiro[4.5]decane-8-carboxylate: A mixture of tert-butyl 2,8-diazaspiro[4.5]decane-8-carboxylate (100 mg, 0.416 mmol, 1.0 equiv.), 2,4-dichloropyrimidine (74.4 mg, 0.499 mmol, 1.2 equiv.) and Cs₂CO₃ (407 mg, 1.25 mmol, 3.0 equiv.) in 1,4-dioxane (2 mL) was stirred for 2 h at 100° C. under air atmosphere. Desired product could be detected by LCMS. The resulting mixture was extracted with EtOAc (3×20 mL). The combined organic layers were washed with water (2×10 mL), dried over anhydrous Na₂SO₄. After filtration, the filtrate was concentrated under reduced pressure. The residue was purified by silica gel column chromatography, eluted with EtOAc/PE=1/1 to afford tert-butyl 2-(2-chloropyrimidin-4-yl)-2,8-diazaspiro[4.5]decane-8-carboxylate (90.0 mg, 61.3%) as a light yellow oil. MS m/z: 353 [M+H]⁺.

Step 2: 2-(2-chloropyrimidin-4-yl)-2,8-diazaspiro[4.5]decane hydrochloride: Followed the general procedure B using tert-butyl 2-(2-chloropyrimidin-4-yl)-2,8-diazaspiro[4.5]decane-8-carboxylate (90.0 mg, 0.255 mmol, 1.0 equiv.) as the starting material to give the crude product 2-(2-chloropyrimidin-4-yl)-2,8-diazaspiro[4.5]decane hydrochloride (70.0 mg). MS m/z: 253 [M+H]⁺.

Step 3: 2-(2-chloropyrimidin-4-yl)-8-(1-(2,2-difluoroethyl)-1H-pyrazolo[3,4-b]pyrazin-6-yl)-2,8-diazaspiro[4.5]decane: Followed the general procedure I using 2-(2-chloropyrimidin-4-yl)-2,8-diazaspiro[4.5]decane hydrochloride (70.0 mg, 0.275 mmol, 1.0 equiv.) and 6-chloro-1-(2,2-difluoroethyl)-1H-pyrazolo[3,4-b]pyrazine (60.1 mg, 0.314 mmol, 1.0 equiv.) as the starting materials to give 2-(2-chloropyrimidin-4-yl)-8-(1-(2,2-difluoroethyl)-1H-pyrazolo[3,4-b]pyrazin-6-yl)-2,8-diazaspiro[4.5]decane (50.0 mg, 41.5%) as a white solid. MS m/z: 435 [M+H]⁺.

Step 4: 8-(1-(2,2-difluoroethyl)-1H-pyrazolo[3,4-b]pyrazin-6-yl)-2-(2-(2,2,2-trifluoroethoxy)pyrimidin-4-yl)-2,8-diazaspiro[4.5]decane: Followed the general procedure E using 2-(2-chloropyrimidin-4-yl)-8-(1-(2,2-difluoroethyl)-1H-pyrazolo[3,4-b]pyrazin-6-yl)-2,8-diazaspiro[4.5]decane (50.0 mg, 0.115 mmol, 1.0 equiv.) and TFE (17.2 mg, 0.172 mmol, 1.5 equiv.) as the starting materials to give 8-(1-(2,2-difluoroethyl)-1H-pyrazolo[3,4-b]pyrazin-6-yl)-2-(2-(2,2,2-trifluoroethoxy)pyrimidin-4-yl)-2,8-diazaspiro[4.5]decane (24.1 mg, 41.9%) as a white solid. ¹H NMR (400 MHz, DMSO-d₆) δ 8.49 (s, 1H), 8.13 (s, 1H), 8.00 (d, J=6.0 Hz, 1H), 6.46 (tt, 1H), 6.27 (d, J=6.1 Hz, 1H), 4.91 (q, J=9.2 Hz, 2H), 4.69 (td, J=15.1, 3.9 Hz, 2H), 3.92-3.82 (m, 2H), 3.76 (d, J=13.7 Hz, 2H), 3.61 (s, 1H), 3.48 (s, 2H), 3.36 (s, 1H), 1.94 (d, J=21.6 Hz, 2H), 1.65 (s, 4H). MS m/z: 499.3 [M+H]⁺.

### Example 739: 8-(1-(2,2-difluoroethyl)-1H-pyrazolo[3,4-b]pyrazin-6-yl)-2-(2-(2,2,2-trifluoroethoxy)pyrimidin-5-yl)-2,8-diazaspiro[4.5]decane

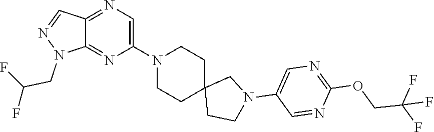 [SMILES: FC(F)Cn1ncc2ncc(N3CCC4(CCN(c5cnc(OCC(F)(F)F)nc5)C4)CC3)nc21]

Step 1: tert-butyl 2-(2-(2,2,2-trifluoroethoxy)pyrimidin-5-yl)-2,8-diazaspiro[4.5]decane-8-carboxylate: Followed the general procedure Y using tert-butyl 2,8-diazaspiro[4.5]decane-8-carboxylate (100 mg, 0.416 mmol, 1.0 equiv.) and 5-bromo-2-(2,2,2-trifluoroethoxy)pyrimidine (117 mg, 0.458 mmol, 1.1 equiv.) as the starting materials, Xphos Pd G3 (35 mg, 41 umol, 0.1 equiv.) as the catalyst to give tert-butyl 2-(2-(2,2,2-trifluoroethoxy)pyrimidin-5-yl)-2,8-diazaspiro[4.5]decane-8-carboxylate (80 mg, 46%) as a colorless oil. MS m/z: 416 [M+H]⁺.

Step 2: 2-(2-(2,2,2-trifluoroethoxy)pyrimidin-5-yl)-2,8-diazaspiro[4.5]decane hydrochloride: Followed the general procedure B using tert-butyl 2-(2-(2,2,2-trifluoroethoxy)pyrimidin-5-yl)-2,8-diazaspiro[4.5]decane-8-carboxylate (80 mg) as the starting material to give the crude product 2-(2-(2,2,2-trifluoroethoxy)pyrimidin-5-yl)-2,8-diazaspiro[4.5]decane hydrochloride (70 mg). MS m/z: 316 [M+H]⁺.

Step 3: 8-(1-(2,2-difluoroethyl)-1H-pyrazolo[3,4-b]pyrazin-6-yl)-2-(2-(2,2,2-trifluoroethoxy)pyrimidin-5-yl)-2,8-diazaspiro[4.5]decane: Followed the general procedure I using 2-(2-(2,2,2-trifluoroethoxy)pyrimidin-5-yl)-2,8-diazaspiro[4.5]decane hydrochloride (70 mg, 0.199 mmol, 1.0 equiv.) and 6-chloro-1-(2,2-difluoroethyl)-1H-pyrazolo[3,4-b]pyrazine (47 mg, 0.219 mol, 1.1 equiv.) as the starting materials to give 8-(1-(2,2-difluoroethyl)-1H-pyrazolo[3,4-b]pyrazin-6-yl)-2-(2-(2,2,2-trifluoroethoxy)pyrimidin-5-yl)-2,8-diazaspiro[4.5]decane (40 mg, 40%) as a white solid. ¹H NMR (400 MHz, DMSO-d₆) δ 8.50 (s, 1H), 8.14 (s, 1H), 8.02 (s, 2H), 6.61-6.28 (m, 1H), 4.91 (q, J=9.0 Hz, 2H), 4.70 (td, J=15.0, 3.8 Hz, 2H), 3.95-3.83 (m, 2H), 3.82-3.69 (m, 2H), 3.39-3.36 (m, 2H), 3.24 (s, 2H), 1.94 (t, J=6.9 Hz, 2H), 1.71-1.63 (m, 4H). MS m/z: 499.25[M+H]⁺.

### Example 740: 8-(1-(2,2-difluoroethyl)-1H-pyrazolo[3,4-b]pyrazin-6-yl)-2-(5-fluoro-4-(trifluoromethyl)pyridin-2-yl)-2,8-diazaspiro[4.5]decane

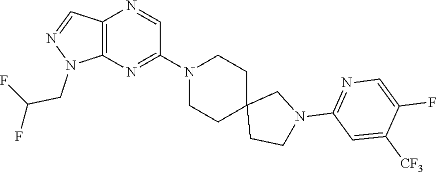 [SMILES: Fc1cnc(N2CCC3(CCN(c4cnc5cnn(CC(F)F)c5n4)CC3)C2)cc1C(F)(F)F]

Step 1: tert-butyl 2-(5-fluoro-4-(trifluoromethyl)pyridin-2-yl)-2,8-diazaspiro[4.5]decane-8-carboxylate: Followed the general procedure Y using tert-butyl 2,8-diazaspiro[4.5]decane-8-carboxylate (130 mg, 0.539 mmol, 1.0 equiv.) and 2-chloro-5-fluoro-4-(trifluoromethyl)pyridine (129 mg, 0.647 mmol, 1.2 equiv.) as the starting materials, Xphos Pd G3 (45.7 mg, 0.054 mmol, 0.1 equiv.) as the catalyst to give tert-butyl 2-(5-fluoro-4-(trifluoromethyl)pyridin-2-yl)-2,8-diazaspiro[4.5]decane-8-carboxylate (100 mg, 45%) as a white solid. MS m/z: 404 [M+H]⁺..

Step 2: 2-(5-fluoro-4-(trifluoromethyl)pyridin-2-yl)-2,8-diazaspiro[4.5]decane hydrochloride: Followed the general procedure B using tert-butyl 2-(5-fluoro-4-(trifluoromethyl)pyridin-2-yl)-2,8-diazaspiro[4.5]decane-8-carboxylate (100 mg) as the starting material to give the crude product 2-(5-fluoro-4-(trifluoromethyl)pyridin-2-yl)-2,8-diazaspiro[4.5]decane hydrochloride (60 mg). MS m/z: 304 [M+H]⁺.

Step 3: 8-(1-(2,2-difluoroethyl)-1H-pyrazolo[3,4-b]pyrazin-6-yl)-2-(5-fluoro-4-(trifluoromethyl)pyridin-2-yl)-2,8-diazaspiro[4.5]decane: Followed the general procedure I using 2-(5-fluoro-4-(trifluoromethyl)pyridin-2-yl)-2,8-diazaspiro[4.5]decane hydrochloride (60 mg, 0.179 mmol, 1.0 equiv.) and 6-chloro-1-(2,2-difluoroethyl)-1H-pyrazolo[3,4-b]pyrazine (47.6 mg, 0.217 mmol, 1.1 equiv.) as the starting materials to give 8-(1-(2,2-difluoroethyl)-1H-pyrazolo[3,4-b]pyrazin-6-yl)-2-(5-fluoro-4-(trifluoromethyl)pyridin-2-yl)-2,8-diazaspiro[4.5]decane (54.8 mg, 57%) as a white solid. ¹H NMR (400 MHz, DMSO-d₆) δ 8.49 (s, 1H), 8.34 (d, J=2.2 Hz, 1H), 8.13 (s, 1H), 6.70 (d, J=4.4 Hz, 1H), 6.44 (tt, J=54.8, 3.8 Hz, 1H), 4.69 (td, J=15.1, 3.8 Hz, 2H), 3.95-3.87 (m, 2H), 3.79-3.68 (m, 2H), 3.53 (t, J=7.0 Hz, 2H), 3.41 (s, 2H), 1.94 (t, J=7.0 Hz, 2H), 1.65 (d, J=9.0 Hz, 4H). MS m/z: 486.3 [M+H]⁺.

### Example 741: 8-(1-(2,2-difluoroethyl)-1H-pyrazolo[3,4-b]pyrazin-6-yl)-2-(5-fluoro-2-(trifluoromethyl)pyridin-4-yl)-2,8-diazaspiro[4.5]decane

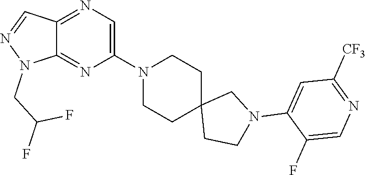 [SMILES: Fc1cnc(C(F)(F)F)cc1N1CCC2(CCN(c3cnc4cnn(CC(F)F)c4n3)CC2)C1]

Step 1: tert-butyl 2-(5-fluoro-2-(trifluoromethyl)pyridin-4-yl)-2,8-diazaspiro[4.5]decane-8-carboxylate: Followed the general procedure Y using tert-butyl 2,8-diazaspiro[4.5]decane-8-carboxylate (100 mg, 0.415 mmol, 1.0 equiv.) and 4-bromo-5-fluoro-2-(trifluoromethyl)pyridine (121 mg, 0.498 mmol, 1.2 equiv.) as the starting materials, XPhos Pd G3 (35.1 mg, 0.239 mmol, 0.1 equiv.) as catalyst to give tert-butyl 2-(5-fluoro-2-(trifluoromethyl)pyridin-4-yl)-2,8-diazaspiro[4.5]decane-8-carboxylate (120 mg, 71.6%) as a light yellow oil. MS m/z: 404 [M+H]⁺.

Step 2: 2-(5-fluoro-2-(trifluoromethyl)pyridin-4-yl)-2,8-diazaspiro[4.5]decane hydrochloride: Followed the general procedure B using tert-butyl 2-(5-fluoro-2-(trifluoromethyl)pyridin-4-yl)-2,8-diazaspiro[4.5]decane-8-carboxylate (120 mg, 0.297 mmol, 1.0 equiv.) as the starting material to give the crude product 2-(5-fluoro-2-(trifluoromethyl)pyridin-4-yl)-2,8-diazaspiro[4.5]decane hydrochloride (100 mg). MS m/z: 304 [M+H]⁺.

Step 3: 8-(1-(2,2-difluoroethyl)-1H-pyrazolo[3,4-b]pyrazin-6-yl)-2-(5-fluoro-2-(trifluoromethyl)pyridin-4-yl)-2,8-diazaspiro[4.5]decane: Followed the general procedure I using 2-(5-fluoro-2-(trifluoromethyl)pyridin-4-yl)-2,8-diazaspiro[4.5]decane hydrochloride (100 mg, 0.329 mmol, 1.0 equiv.) and 6-chloro-1-(2,2-difluoroethyl)-1H-pyrazolo[3,4-b]pyrazine (72.0 mg, 0.329 mmol, 1.0 equiv.) as the starting materials to give 8-(1-(2,2-difluoroethyl)-1H-pyrazolo[3,4-b]pyrazin-6-yl)-2-(5-fluoro-2-(trifluoromethyl)pyridin-4-yl)-2,8-diazaspiro[4.5]decane (29.5 mg, 18.4%) as a white solid. ¹H NMR (400 MHz, DMSO-d₆) δ 8.49 (s, 1H), 8.26 (d, J=5.7 Hz, 1H), 8.13 (s, 1H), 7.01 (d, J=7.3 Hz, 1H), 6.44 (tt, J=55.0, 3.8 Hz, 1H), 4.69 (td, J=15.0, 3.8 Hz, 2H), 3.90-3.65 (m, 6H), 3.49 (s, 2H), 1.92 (t, J=7.0 Hz, 2H), 1.74-1.60 (m, 4H). MS m/z: 486.25 [M+H]⁺.

### Example 742: 8-(1-(2,2-difluoroethyl)-1H-pyrazolo[3,4-b]pyrazin-6-yl)-2-(5-fluoro-6-(trifluoromethyl)pyridin-3-yl)-2,8-diazaspiro[4.5]decane

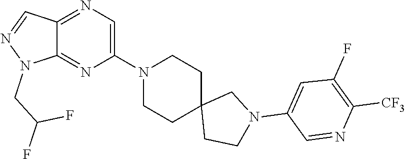 [SMILES: Fc1cc(N2CCC3(CCN(c4cnc5cnn(CC(F)F)c5n4)CC3)C2)cnc1C(F)(F)F]

Step 1: tert-butyl 2-(5-fluoro-6-(trifluoromethyl)pyridin-3-yl)-2,8-diazaspiro[4.5]decane-8-carboxylate: Followed the general procedure I using 5-bromo-3-fluoro-2-(trifluoromethyl)pyridine (200 mg, 819 μmol 1.0 equiv.) and tert-butyl 2,8-diazaspiro[4.5]decane-8-carboxylate (196 mg, 819 μmol, 1.0 equiv.) as the starting materials to give tert-butyl 2-(5-fluoro-6-(trifluoromethyl)pyridin-3-yl)-2,8-diazaspiro[4.5]decane-8-carboxylate (200 mg, 60%) as a white solid. MS m/z: 404 [M+H]⁺.

Step 2: 2-(5-fluoro-6-(trifluoromethyl)pyridin-3-yl)-2,8-diazaspiro[4.5]decane: Followed the general procedure H using tert-butyl 2-(5-fluoro-6-(trifluoromethyl)pyridin-3-yl)-2,8-diazaspiro[4.5]decane-8-carboxylate (200 mg) as the starting material to give the crude product 2-(5-fluoro-6-(trifluoromethyl)pyridin-3-yl)-2,8-diazaspiro[4.5]decane (150 mg). MS m/z: 304 [M+H]⁺.

Step 3: 8-(1-(2,2-difluoroethyl)-1H-pyrazolo[3,4-b]pyrazin-6-yl)-2-(5-fluoro-6-(trifluoromethyl)pyridin-3-yl)-2,8-diazaspiro[4.5]decane: Followed the general procedure I using 2-(5-fluoro-6-(trifluoromethyl)pyridin-3-yl)-2,8-diazaspiro[4.5]decane (90 mg, 296 μmol, 1.0 equiv.) and 6-chloro-1-(2,2-difluoroethyl)-1H-pyrazolo[3,4-b]pyrazine (65 mg, 296 μmol, 1.0 equiv.) as the starting materials to give 8-(1-(2,2-difluoroethyl)-1H-pyrazolo[3,4-b]pyrazin-6-yl)-2-(5-fluoro-6-(trifluoromethyl)pyridin-3-yl)-2,8-diazaspiro[4.5]decane (35 mg, 24%) as a white solid. ¹H NMR (400 MHz, DMSO-d₆) δ 8.50 (s, 1H), 8.14 (s, 1H), 7.88 (t, J=2.1 Hz, 1H), 6.97 (dd, J=14.2, 2.2 Hz, 1H), 6.44 (tt, J=54.9, 3.8 Hz, 1H), 4.70 (td, J=15.0, 3.8 Hz, 2H), 3.92 (dt, J=13.6, 5.4 Hz, 2H), 3.79-3.68 (m, 2H), 3.48 (t, J=7.0 Hz, 2H), 3.36 (s, 2H), 1.97 (t, J=7.0 Hz, 2H), 1.73-1.59 (m, 4H). MS m/z: 486.10 [M+H]⁺.

### Example 743: 8-(1-(2,2-difluoroethyl)-1H-pyrazolo[3,4-b]pyrazin-6-yl)-2-(3-fluoro-5-(trifluoromethyl)pyridin-2-yl)-2,8-diazaspiro[4.5]decane

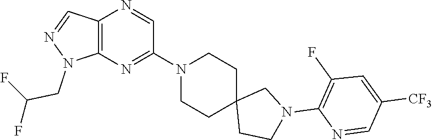 [SMILES: Fc1cc(C(F)(F)F)cnc1N1CCC2(CCN(c3cnc4cnn(CC(F)F)c4n3)CC2)C1]

Step 1: tert-butyl 2-(3-fluoro-5-(trifluoromethyl)pyridin-2-yl)-2,8-diazaspiro[4.5]decane-8-carboxylate: Followed the general procedure E using tert-butyl 2,8-diazaspiro[4.5]decane-8-carboxylate (150 mg, 0.622 mmol, 1.0 equiv.) and 2,3-difluoro-5-(trifluoromethyl)pyridine (137 mg, 0.747 mmol, 1.2 equiv.) as the starting materials to give tert-butyl 2-(3-fluoro-5-(trifluoromethyl)pyridin-2-yl)-2,8-diazaspiro[4.5]decane-8-carboxylate (120 mg, 47.7%) as a light yellow oil. MS m/z: 404 [M+H]⁺.

Step 2: 2-(3-fluoro-5-(trifluoromethyl)pyridin-2-yl)-2,8-diazaspiro[4.5]decane hydrochloride: Followed the general procedure B using tert-butyl 2-(3-fluoro-5-(trifluoromethyl)pyridin-2-yl)-2,8-diazaspiro[4.5]decane-8-carboxylate (120 mg, 0.297 mmol, 1.0 equiv.) as the starting material to give the crude product 2-(3-fluoro-5-(trifluoromethyl)pyridin-2-yl)-2,8-diazaspiro[4.5]decane hydrochloride (90.0 mg). MS m/z: 304 [M+H]⁺.

Step 3: 8-(1-(2,2-difluoroethyl)-1H-pyrazolo[3,4-b]pyrazin-6-yl)-2-(3-fluoro-5-(trifluoromethyl)pyridin-2-yl)-2,8-diazaspiro[4.5]decane: Followed the general procedure I using 2-(3-fluoro-5-(trifluoromethyl)pyridin-2-yl)-2,8-diazaspiro[4.5]decane hydrochloride (90.0 mg, 0.296 mmol, 1.0 equiv.) and 6-chloro-1-(2,2-difluoroethyl)-1H-pyrazolo[3,4-b]pyrazine (64.8 mg, 0.296 mmol, 1.0 equiv.) as the starting materials to give 8-(1-(2,2-difluoroethyl)-1H-pyrazolo[3,4-b]pyrazin-6-yl)-2-(3-fluoro-5-(trifluoromethyl)pyridin-2-yl)-2,8-diazaspiro[4.5]decane (75.1 mg, 52.1%) as a white solid. ¹H NMR (400 MHz, DMSO-d₆) δ 8.48 (s, 1H), 8.24 (p, J=1.2 Hz, 1H), 8.13 (s, 1H), 7.79 (dd, J=13.6, 2.0 Hz, 1H), 6.44 (tt, J=54.9, 3.8 Hz, 1H), 4.69 (td, J=15.0, 3.8 Hz, 2H), 3.88-3.72 (m, 6H), 3.58 (d, J=2.4 Hz, 2H), 1.92 (t, J=7.1 Hz, 2H), 1.67 (q, J=6.1 Hz, 4H). MS m/z: 486.4 [M+H]⁺.

### Example 745: 8-(1-(2,2-difluoroethyl)-1H-pyrazolo[3,4-b]pyrazin-6-yl)-7-methyl-2-(4-(trifluoromethyl)pyridin-2-yl)-2,8-diazaspiro[4.5]decane

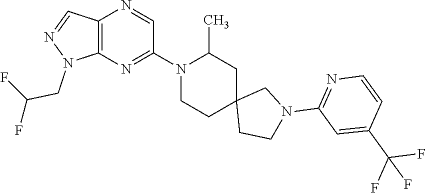 [SMILES: CC1CC2(CCN(c3cc(C(F)(F)F)ccn3)C2)CCN1c1cnc2cnn(CC(F)F)c2n1]

Step 1: tert-butyl 7-methyl-3-oxo-2-(4-(trifluoromethyl)pyridin-2-yl)-2,8-diazaspiro[4.5]decane-8-carboxylate: Followed the general procedure E using tert-butyl 7-methyl-3-oxo-2,8-diazaspiro[4.5]decane-8-carboxylate (100 mg, 0.373 mmol, 1.0 equiv.) and 2-fluoro-4-(trifluoromethyl)pyridine (61 mg, 0.373 mmol, 1.0 equiv.) as the starting materials to give tert-butyl 7-methyl-3-oxo-2-(4-(trifluoromethyl)pyridin-2-yl)-2,8-diazaspiro[4.5]decane-8-carboxylate (140 mg, 91%) as a colorless oil. MS m/z: 414 [M+H]⁺.

Step 2: tert-butyl 7-methyl-2-(4-(trifluoromethyl)pyridin-2-yl)-2,8-diazaspiro[4.5]decane-8-carboxylate: Followed the general procedure AH using tert-butyl 7-methyl-3-oxo-2-(4-(trifluoromethyl)pyridin-2-yl)-2,8-diazaspiro[4.5]decane-8-carboxylate (120 mg, 0.29 mmol, 1.0 equiv.) as the starting material to give tert-butyl 7-methyl-2-(4-(trifluoromethyl)pyridin-2-yl)-2,8-diazaspiro[4.5]decane-8-carboxylate (50 mg, 43%) as a colorless oil. MS m/z: 400 [M+H]⁺.

Step 3: 7-methyl-2-(4-(trifluoromethyl)pyridin-2-yl)-2,8-diazaspiro[4.5]decane hydrochloride: Followed the general procedure B using tert-butyl 7-methyl-2-(4-(trifluoromethyl)pyridin-2-yl)-2,8-diazaspiro[4.5]decane-8-carboxylate (50 mg) as the starting material to give the crude product 7-methyl-2-(4-(trifluoromethyl)pyridin-2-yl)-2,8-diazaspiro[4.5]decane hydrochloride (40 mg). MS m/z: 300 [M+H]⁺.

Step 4: 8-(1-(2,2-difluoroethyl)-1H-pyrazolo[3,4-b]pyrazin-6-yl)-7-methyl-2-(4-(trifluoromethyl)pyridin-2-yl)-2,8-diazaspiro[4.5]decane: Followed the general procedure I using 7-methyl-2-(4-(trifluoromethyl)pyridin-2-yl)-2,8-diazaspiro[4.5]decane hydrochloride (40 mg, 0.119 mmol, 1.0 equiv.) and 6-chloro-1-(2,2-difluoroethyl)-1H-pyrazolo[3,4-b]pyrazine (28 mg, 0.131 mmol, 1.1 equiv.) as the starting materials to give 8-(1-(2,2-difluoroethyl)-1H-pyrazolo[3,4-b]pyrazin-6-yl)-7-methyl-2-(4-(trifluoromethyl)pyridin-2-yl)-2,8-diazaspiro[4.5]decane (4 mg, 7%) as a brown solid. ¹H NMR (400 MHz, DMSO-d₆) δ 8.42 (s, 1H), 8.26 (d, J=5.2 Hz, 1H), 8.12 (s, 1H), 6.80-6.76 (m, 1H), 6.62 (s, 1H), 6.43 (tt, J=54.9, 3.8 Hz, 1H), 4.86-4.76 (m, 1H), 4.69 (td, J=15.1, 3.8 Hz, 2H), 4.39 (dt, J=14.1, 3.9 Hz, 1H), 3.68-3.59 (m, 1H), 3.59-3.49 (m, 1H), 3.31-3.17 (m, 3H), 2.35-2.25 (m, 1H), 2.03-1.90 (m, 2H), 1.84-1.76 (m, 1H), 1.74-1.67 (m, 1H), 1.62-1.51 (m, 1H), 1.28 (d, J=6.8 Hz, 3H). MS m/z: 482.25 [M+H]⁺.

### Example 746: 8-(1-(2,2-difluoroethyl)-1H-pyrazolo[3,4-b]pyrazin-6-yl)-6-methyl-2-(4-(trifluoromethyl)pyridin-2-yl)-2,8-diazaspiro[4.5]decane

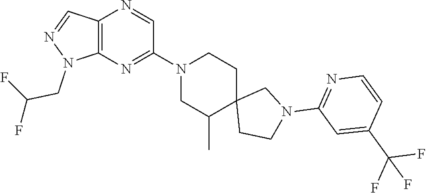 [SMILES: CC1CN(c2cnc3cnn(CC(F)F)c3n2)CCC12CCN(c1cc(C(F)(F)F)ccn1)C2]

Step 1: tert-butyl 4-(2-ethoxy-2-oxoethylidene)-3-methylpiperidine-1-carboxylate: Followed the general procedure Z using tert-butyl 3-methyl-4-oxopiperidine-1-carboxylate (2 g, 9.39 mmol, 1.0 equiv.) and ethyl 2-(diethoxyphosphoryl)acetate (2.1 g, 9.39 mol, 1.0 equiv.) as the starting materials to give tert-butyl 4-(2-ethoxy-2-oxoethylidene)-3-methylpiperidine-1-carboxylate (1.2 g, 45%) as a white solid. MS m/z: 284 [M+H]⁺.

Step 2: tert-butyl 4-(2-methoxy-2-oxoethyl)-3-methyl-4-(nitromethyl)piperidine-1-carboxylate: Followed the general procedure AA using tert-butyl 4-(2-ethoxy-2-oxoethylidene)-3-methylpiperidine-1-carboxylate (1.2 g, 4.24 mmol, 1.0 equiv.) as the starting material to give tert-butyl 4-(2-methoxy-2-oxoethyl)-3-methyl-4-(nitromethyl)piperidine-1-carboxylate (800 mg, 57%) as a white solid. MS m/z: 331 [M+H]⁺.

Step 3: tert-butyl 6-methyl-3-oxo-2,8-diazaspiro[4.5]decane-8-carboxylate: Followed the general procedure AB using tert-butyl 4-(2-methoxy-2-oxoethyl)-3-methyl-4-(nitromethyl)piperidine-1-carboxylate (800 mg, 2.42 mmol, 1.0 equiv.) as the starting material to give tert-butyl 6-methyl-3-oxo-2,8-diazaspiro[4.5]decane-8-carboxylate (460 mg, 71%) as a white solid. MS m/z: 269 [M+H]⁺.

Step 4: tert-butyl 6-methyl-3-oxo-2-(4-(trifluoromethyl)pyridin-2-yl)-2,8-diazaspiro[4.5]decane-8-carboxylate: Followed the general procedure E using tert-butyl 6-methyl-3-oxo-2,8-diazaspiro[4.5]decane-8-carboxylate (460 mg, 1.72 mmol, 1.0 equiv.) and 2-fluoro-4-(trifluoromethyl)pyridine (284 mg, 1.72 mmol, 1.0 equiv.) as the starting materials to give tert-butyl 6-methyl-3-oxo-2-(4-(trifluoromethyl)pyridin-2-yl)-2,8-diazaspiro[4.5]decane-8-carboxylate (200 mg, 28%) as a white solid. MS m/z: 414 [M+H]⁺.

Step 5: tert-butyl 6-methyl-2-(4-(trifluoromethyl)pyridin-2-yl)-2,8-diazaspiro[4.5]decane-8-carboxylate: Followed the general procedure AH using tert-butyl 6-methyl-3-oxo-2-(4-(trifluoromethyl)pyridin-2-yl)-2,8-diazaspiro[4.5]decane-8-carboxylate (200 mg, 0.484 mmol) as the starting material, BH₃-DMS (10 M, 0.1 mL, 1.5 equiv.) as the reductant to give tert-butyl 6-methyl-2-(4-(trifluoromethyl)pyridin-2-yl)-2,8-diazaspiro[4.5]decane-8-carboxylate (90 mg, 46%) as a white solid. MS m/z: 400 [M+H]⁺.

Step 6: 6-methyl-2-(4-(trifluoromethyl)pyridin-2-yl)-2,8-diazaspiro[4.5]decane hydrochloride: Followed the general procedure B using tert-butyl 6-methyl-2-(4-(trifluoromethyl)pyridin-2-yl)-2,8-diazaspiro[4.5]decane-8-carboxylate (90 mg) as the starting material to give the crude product 6-methyl-2-(4-(trifluoromethyl)pyridin-2-yl)-2,8-diazaspiro[4.5]decane hydrochloride (65 mg). MS m/z: 299 [M+H]⁺.

Step 7: 8-(1-(2,2-difluoroethyl)-1H-pyrazolo[3,4-b]pyrazin-6-yl)-6-methyl-2-(4-(trifluoromethyl)pyridin-2-yl)-2,8-diazaspiro[4.5]decane: Followed the general procedure I using 6-methyl-2-(4-(trifluoromethyl)pyridin-2-yl)-2,8-diazaspiro[4.5]decane hydrochloride (65 mg, 0.217 mmol, 1.0 equiv.) and 6-chloro-1-(2,2-difluoroethyl)-1H-pyrazolo[3,4-b]pyrazine (48 mg, 0.217 mol, 1.0 equiv.) as the starting materials to give 8-(1-(2,2-difluoroethyl)-1H-pyrazolo[3,4-b]pyrazin-6-yl)-6-methyl-2-(4-(trifluoromethyl)pyridin-2-yl)-2,8-diazaspiro[4.5]decane (52.8 mg, 50%) as an off-white solid. ¹H NMR (400 MHz, DMSO-d₆) δ 8.49 (s, 1H), 8.29 (d, J=5.2 Hz, 1H), 8.11 (s, 1H), 6.79 (dd, J=5.2, 1.4 Hz, 1H), 6.71 (d, J=11.0 Hz, 1H), 6.43 (tt, J=55.0, 3.8 Hz, 1H), 4.75-4.62 (m, 2H), 3.93-3.81 (m, 1H), 3.81-3.64 (m, 3H), 3.63-3.45 (m, 3H), 3.38 (d, J=10.9 Hz, 1H), 2.07-1.93 (m, 1H), 1.92-1.69 (m, 3H), 1.62-1.51 (m, 1H), 1.00-0.92 (m, 3H). MS m/z: 482.2 [M+H]⁺.

### Example 747: 8-(1-(2,2-difluoroethyl)-1H-pyrazolo[3,4-b]pyrazin-6-yl)-1-methyl-2-(4-(trifluoromethyl)pyridin-2-yl)-2,8-diazaspiro[4.5]decane

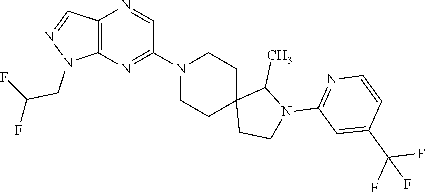 [SMILES: CC1N(c2cc(C(F)(F)F)ccn2)CCC12CCN(c1cnc3cnn(CC(F)F)c3n1)CC2]

Step 1: tert-butyl 4-(2-ethoxy-2-oxoethyl)-4-(1-nitroethyl)piperidine-1-carboxylate: A solution of tert-butyl 4-(2-ethoxy-2-oxoethylidene)piperidine-1-carboxylate (3 g, 11.1 mmol, 1.0 equiv.), TBAF (55.7 mL, 55.6 mmol, 5.0 equiv.) and nitroethane (2.09 g, 27.8 mmol, 2.5 equiv.) in THF (100 mL) was stirred for overnight at 60° C. The resulting mixture was concentrated under vacuum. The resulting mixture was extracted with EtOAc (200 mL). The combined organic layers were washed with water (3×40 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 100% gradient in 40 min; detector, UV 254 nm. This resulted in tert-butyl 4-(2-ethoxy-2-oxoethyl)-4-(1-nitroethyl)piperidine-1-carboxylate (1.89 g, 50%) as a colorless oil. MS m/z: 345 [M+H]⁺.

Step 2: tert-butyl 1-methyl-3-oxo-2,8-diazaspiro[4.5]decane-8-carboxylate: Followed the general procedure AB using tert-butyl 4-(2-ethoxy-2-oxoethyl)-4-(1-nitroethyl)piperidine-1-carboxylate (1.89 g, 5.57 mmol, 1.0 equiv.) as the starting material to give tert-butyl 1-methyl-3-oxo-2,8-diazaspiro[4.5]decane-8-carboxylate (1 g, 68%) as a colorless oil. MS m/z: 269 [M+H]⁺.

Step 3: tert-butyl 1-methyl-2,8-diazaspiro[4.5]decane-8-carboxylate: Followed the general procedure AH using tert-butyl 1-methyl-3-oxo-2,8-diazaspiro[4.5]decane-8-carboxylate (400 mg, 1.49 mmol, 1.0 equiv.) as the starting material to give tert-butyl 1-methyl-2,8-diazaspiro[4.5]decane-8-carboxylate (140 mg, 37%) as a colorless oil. MS m/z: 255 [M+H]⁺.

Step 4: tert-butyl 1-methyl-2-(4-(trifluoromethyl)pyridin-2-yl)-2,8-diazaspiro[4.5]decane-8-carboxylate: Followed the general procedure I using tert-butyl 1-methyl-2,8-diazaspiro[4.5]decane-8-carboxylate (70 mg, 0.27 mmol, 1.0 equiv.) and 2-fluoro-4-(trifluoromethyl)pyridine (68 mg, 0.41 mmol, 1.5 equiv.) as the starting materials to give tert-butyl 1-methyl-2-(4-(trifluoromethyl)pyridin-2-yl)-2,8-diazaspiro[4.5]decane-8-carboxylate (40 mg, 37%) as a colorless oil. MS m/z: 400 [M+H]⁺.

Step 5: 1-methyl-2-(4-(trifluoromethyl)pyridin-2-yl)-2,8-diazaspiro[4.5]decane hydrochloride: Followed the general procedure B using tert-butyl 1-methyl-2-(4-(trifluoromethyl)pyridin-2-yl)-2,8-diazaspiro[4.5]decane-8-carboxylate as the starting material (40 mg) to give the crude product 1-methyl-2-(4-(trifluoromethyl)pyridin-2-yl)-2,8-diazaspiro[4.5]decane hydrochloride (30 mg). MS m/z: 300 [M+H]⁺.

Step 6: 8-(1-(2,2-difluoroethyl)-1H-pyrazolo[3,4-b]pyrazin-6-yl)-1-methyl-2-(4-(trifluoromethyl)pyridin-2-yl)-2,8-diazaspiro[4.5]decane: Followed the general procedure I using 1-methyl-2-(4-(trifluoromethyl)pyridin-2-yl)-2,8-diazaspiro[4.5]decane hydrochloride (30 mg, 0.09 mmol, 1.0 equiv.) and 6-chloro-1-(2,2-difluoroethyl)-1H-pyrazolo[3,4-b]pyrazine (21 mg, 0.1 mmol, 1.1 equiv.) as the starting materials to give 8-(1-(2,2-difluoroethyl)-1H-pyrazolo[3,4-b]pyrazin-6-yl)-1-methyl-2-(4-(trifluoromethyl)pyridin-2-yl)-2,8-diazaspiro[4.5]decane (10 mg, 23%) as a white solid. ¹H NMR (400 MHz, DMSO-d₆) δ 8.47 (s, 1H), 8.28 (d, J=5.1 Hz, 1H), 8.13 (s, 1H), 6.76 (d, J=5.3 Hz, 1H), 6.66 (s, 1H), 6.59-6.27 (m, 1H), 4.68 (td, J=15.0, 3.8 Hz, 2H), 4.13 (d, J=6.4 Hz, 1H), 3.94-3.83 (m, 2H), 3.81-3.64 (m, 2H), 3.55-3.46 (m, 1H), 3.45-3.37 (m, 1H), 2.10-2.00 (m, 1H), 1.99-1.90 (m, 1H), 1.85-1.73 (m, 1H), 1.69-1.59 (m, 1H), 1.57-1.47 (m, 2H), 1.09 (d, J=6.3 Hz, 3H). MS m/z: 482.15 [M+H]⁺.

### Example 748: 8-(1-(2,2-difluoroethyl)-1H-pyrazolo[3,4-b]pyrazin-6-yl)-4-methyl-2-(4-(trifluoromethyl)pyridin-2-yl)-2,8-diazaspiro[4.5]decane

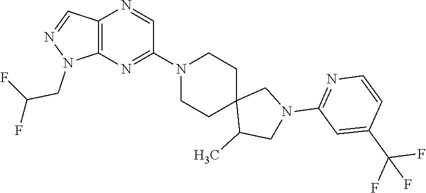 [SMILES: CC1CN(c2cc(C(F)(F)F)ccn2)CC12CCN(c1cnc3cnn(CC(F)F)c3n1)CC2]

Step 1: tert-butyl 3-oxo-2-(4-(trifluoromethyl)pyridin-2-yl)-2,8-diazaspiro[4.5]decane-8-carboxylate: Followed the general procedure K using tert-butyl 3-oxo-2,8-diazaspiro[4.5]decane-8-carboxylate (300 mg, 1.18 mmol, 1.0 equiv.) and 2-fluoro-4-(trifluoromethyl)pyridine (214 mg, 1.30 mmol, 1.1 equiv.) as the starting materials to give tert-butyl 3-oxo-2-(4-(trifluoromethyl)pyridin-2-yl)-2,8-diazaspiro[4.5]decane-8-carboxylate (220 mg, 46%) as a yellow oil. MS m/z: 400 [M+H]⁺.

Step 2: tert-butyl 4-methyl-3-oxo-2-(4-(trifluoromethyl)pyridin-2-yl)-2,8-diazaspiro[4.5]decane-8-carboxylate: Followed the general procedure AC using tert-butyl 3-oxo-2-(4-(trifluoromethyl)pyridin-2-yl)-2,8-diazaspiro[4.5]decane-8-carboxylate (200 mg, 0.501 mmol, 1.0 equiv.) and iodomethane (106 mg, 0.752 mmol, 1.2 equiv.) as the starting materials to give tert-butyl 4-methyl-3-oxo-2-(4-(trifluoromethyl)pyridin-2-yl)-2,8-diazaspiro[4.5]decane-8-carboxylate (120 mg, 58%) as a yellow solid. MS m/z: 414 [M+H]⁺.

Step 3: tert-butyl 4-methyl-2-(4-(trifluoromethyl)pyridin-2-yl)-2,8-diazaspiro[4.5]decane-8-carboxylate: Followed the general procedure AH using tert-butyl 4-methyl-3-oxo-2-(4-(trifluoromethyl)pyridin-2-yl)-2,8-diazaspiro[4.5]decane-8-carboxylate (120 mg, 0.501 mmol, 1.0 equiv.) as the starting material to give tert-butyl 4-methyl-2-(4-(trifluoromethyl)pyridin-2-yl)-2,8-diazaspiro[4.5]decane-8-carboxylate (55 mg, 46%) as a yellow solid. MS m/z: 400 [M+H]⁺.

Step 4: 4-methyl-2-(4-(trifluoromethyl)pyridin-2-yl)-2,8-diazaspiro[4.5]decane hydrochloride: Followed the general procedure B using tert-butyl 4-methyl-2-(4-(trifluoromethyl)pyridin-2-yl)-2,8-diazaspiro[4.5]decane-8-carboxylate (55 mg) as the starting material to give the crude product 4-methyl-2-(4-(trifluoromethyl)pyridin-2-yl)-2,8-diazaspiro[4.5]decane hydrochloride (40 mg) as a yellow solid. MS m/z: 300 [M+H]⁺.

Step 5: 8-(1-(2,2-difluoroethyl)-1H-pyrazolo[3,4-b]pyrazin-6-yl)-4-metyl-2-(4-(trifluoromethyl)pyridin-2-yl)-2,8-diazaspiro[4.5]decane: Followed the general procedure I using 4-methyl-2-(4-(trifluoromethyl)pyridin-2-yl)-2,8-diazaspiro[4.5]decane hydrochloride (40 mg, 0.134 mmol, 1.0 equiv.) and 6-chloro-1-(2,2-difluoroethyl)-1H-pyrazolo[3,4-b]pyrazine (32 mg, 0.147 mmol, 1.1 equiv.) as the starting materials to give 8-(1-(2,2-difluoroethyl)-1H-pyrazolo[3,4-b]pyrazin-6-yl)-4-methyl-2-(4-(trifluoromethyl)pyridin-2-yl)-2,8-diazaspiro[4.5]decane (32.5 mg, 50%) as a white solid. ¹H NMR (400 MHz, DMSO-d₆) δ 8.48 (s, 1H), 8.28 (d, J=5.2 Hz, 1H), 8.13 (s, 1H), 6.80-6.77 (m, 1H), 6.71 (s, 1H), 6.37 (dt, J=54.8, 3.8 Hz, 1H), 4.69 (td, J=15.0, 3.8 Hz, 2H), 4.36 (t, J=12.6 Hz, 2H), 3.86-3.69 (m, 2H), 3.49-3.34 (m, 1H), 3.30-3.20 (m, 2H), 3.19-3.11 (m, 1H), 2.13 (q, J=7.2 Hz, 1H), 1.77-1.67 (m, 1H), 1.62-1.44 (m, 3H), 0.95 (d, J=6.9 Hz, 3H). MS m/z: 482.15 [M+H]⁺.

### Example 749: 8-(1-(2,2-difluoroethyl)-1H-pyrazolo[3,4-b]pyrazin-6-yl)-3-methyl-2-(4-(trifluoromethyl)pyridin-2-yl)-2,8-diazaspiro[4.5]decane

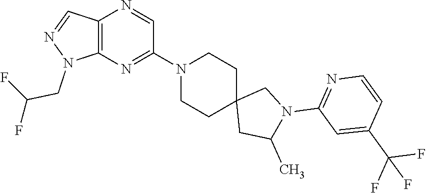 [SMILES: CC1CC2(CCN(c3cnc4cnn(CC(F)F)c4n3)CC2)CN1c1cc(C(F)(F)F)ccn1]

Step 1: tert-butyl 4-(2-oxopropylidene)piperidine-1-carboxylate: Followed the general procedure Z using tert-butyl 4-oxopiperidine-1-carboxylate (1 g, 5.03 mmol, 1.0 equiv.) and dimethyl (2-oxopropyl)phosphonate (1.1 g, 4.77 mmol, 0.95 equiv.) as the starting materials to give tert-butyl 4-(2-oxopropylidene)piperidine-1-carboxylate (1 g, 83%) as a yellow oil. MS m/z: 240 [M+H]⁺.

Step 2: tert-butyl 4-(nitromethyl)-4-(2-oxopropyl)piperidine-1-carboxylate: Followed the general procedure AA using tert-butyl 3-oxo-2-(4-(trifluoromethyl)pyridin-2-yl)-2,8-diazaspiro[4.5]decane-8-carboxylate (1000 mg, 4.18 mmol) as the starting material to give tert-butyl 4-(nitromethyl)-4-(2-oxopropyl)piperidine-1-carboxylate (200 mg, 16%) as a yellow solid. MS m/z: 301 [M+H]⁺.

Step 3: tert-butyl 3-methyl-2,8-diazaspiro[4.5]dec-2-ene-8-carboxylate: To a stirred solution of tert-butyl 4-(nitromethyl)-4-(2-oxopropyl)piperidine-1-carboxylate (200 mg, 0.667 mmol, 1.0 equiv.) and Zn (427 mg, 6.67 mmol, 10 equiv.) in i-PrOH (2 mL) was added HCl (1 mol/L, 2 mL) at room temperature. The resulting mixture was stirred for overnight at 60° C. Desired product could be detected by LCMS. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:1) to afford tert-butyl 3-methyl-2,8-diazaspiro[4.5]dec-2-ene-8-carboxylate (130 mg, 77%) as a colorless oil. MS m/z: 253 [M+H]⁺.

Step 4: tert-butyl 3-methyl-2,8-diazaspiro[4.5]decane-8-carboxylate: Followed the general procedure Q using tert-butyl 3-methyl-2,8-diazaspiro[4.5]dec-2-ene-8-carboxylate (130 mg, 0.516 mmol, 1.0 equiv.) as the starting material to give tert-butyl 3-methyl-2,8-diazaspiro[4.5]decane-8-carboxylate (110 mg, 76%) as a yellow solid. MS m/z: 255 [M+H]⁺.

Step 5: tert-butyl 3-methyl-2-(4-(trifluoromethyl)pyridin-2-yl)-2,8-diazaspiro[4.5]decane-8-carboxylate: Followed the general procedure I using tert-butyl 3-methyl-2,8-diazaspiro[4.5]decane-8-carboxylate (110 mg, 0.433 mmol, 1.0 equiv.) and 2-fluoro-4-(trifluoromethyl)pyridine (78.6 mg, 0.476 mmol, 1.1 equiv.) as the starting materials to give tert-butyl 3-methyl-2-(4-(trifluoromethyl)pyridin-2-yl)-2,8-diazaspiro[4.5]decane-8-carboxylate (100 mg, 58%) as a yellow oil. MS m/z: 400 [M+H]⁺.

Step 6: 3-methyl-2-(4-(trifluoromethyl)pyridin-2-yl)-2,8-diazaspiro[4.5]decane hydrochloride: Followed the general procedure B using tert-butyl 3-methyl-2,8-diazaspiro[4.5]decane-8-carboxylate (100 mg) as the starting material to give the crude product 3-methyl-2-(4-(trifluoromethyl)pyridin-2-yl)-2,8-diazaspiro[4.5]decane hydrochloride (80 mg) as a yellow solid. MS m/z: 300 [M+H]⁺.

Step 7: 8-(1-(2,2-difluoroethyl)-1H-pyrazolo[3,4-b]pyrazin-6-yl)-3-methyl-2-(4-(trifluoromethyl)pyridin-2-yl)-2,8-diazaspiro[4.5]decane: Followed the general procedure I using 3-methyl-2-(4-(trifluoromethyl)pyridin-2-yl)-2,8-diazaspiro[4.5]decane hydrochloride (80 mg, 0.268 mmol, 1.0 equiv.) and 6-chloro-1-(2,2-difluoroethyl)-1H-pyrazolo[3,4-b]pyrazine (64.2 mg, 0.294 mmol, 1.1 equiv.) as the starting materials to give 8-(1-(2,2-difluoroethyl)-1H-pyrazolo[3,4-b]pyrazin-6-yl)-3-methyl-2-(4-(trifluoromethyl)pyridin-2-yl)-2,8-diazaspiro[4.5]decane (44.6 mg, 34%) as a white solid. ¹H NMR (400 MHz, DMSO-d₆) δ 8.47 (s, 1H), 8.30 (d, J=5.2 Hz, 1H), 8.13 (s, 1H), 6.79 (dd, J=5.1, 1.4 Hz, 1H), 6.74 (s, 1H), 6.43 (tt, J=54.9, 3.8 Hz, 1H), 4.68 (td, J=15.0, 3.8 Hz, 2H), 4.18 (q, J=6.7 Hz, 1H), 3.95-3.84 (m, 2H), 3.76-3.64 (m, 3H), 3.38 (d, J=10.9 Hz, 1H), 2.26 (dd, J=12.8, 7.8 Hz, 1H), 1.81-1.67 (m, 2H), 1.63-1.56 (m, 1H), 1.51 (t, J=5.8 Hz, 2H), 1.29 (d, J=6.0 Hz, 3H). MS m/z: 482.1 [M+H]⁺.

### Example 750: 8-(1-(2-methoxyethyl)-1H-pyrazolo[3,4-b]pyrazin-6-yl)-2-(4-(trifluoromethyl)pyridin-2-yl)-2,8-diazaspiro[4.5]decane

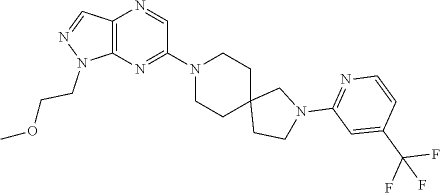 [SMILES: COCCn1ncc2ncc(N3CCC4(CCN(c5cc(C(F)(F)F)ccn5)C4)CC3)nc21]

Step 1: tert-butyl 2-(4-(trifluoromethyl)pyridin-2-yl)-2,8-diazaspiro[4.5]decane-8-carboxylate: Followed the general procedure K using tert-butyl 2,8-diazaspiro[4.5]decane-8-carboxylate (500 mg, 2.07 mmol, 1.0 equiv.) and 2-bromo-4-(trifluoromethyl)pyridine (563 mg, 2.49 mmol, 1.2 equiv.) as the starting materials to give tert-butyl 2-(4-(trifluoromethyl)pyridin-2-yl)-2,8-diazaspiro[4.5]decane-8-carboxylate (360 mg, 45.0%) as a light yellow solid. MS m/z: 386 [M+H]⁺.

Step 2: 2-(4-(trifluoromethyl)pyridin-2-yl)-2,8-diazaspiro[4.5]decane hydrochloride: Followed the general procedure B using tert-butyl 2-(4-(trifluoromethyl)pyridin-2-yl)-2,8-diazaspiro[4.5]decane-8-carboxylate (360 mg, 0.933 mmol, 1.0 equiv.) as the starting material to give the crude product 2-(4-(trifluoromethyl)pyridin-2-yl)-2,8-diazaspiro[4.5]decane hydrochloride (320 mg). MS m/z: 286 [M+H]⁺.

Step 3: 8-(1-(2-methoxyethyl)-1H-pyrazolo[3,4-b]pyrazin-6-yl)-2-(4-(trifluoromethyl)pyridin-2-yl)-2,8-diazaspiro[4.5]decane: Followed the general procedure I using 2-(4-(trifluoromethyl)pyridin-2-yl)-2,8-diazaspiro[4.5]decane hydrochloride (100 mg, 0.350 mmol, 1.0 equiv.) and 6-chloro-1-(2-methoxyethyl)-1H-pyrazolo[3,4-b]pyrazine (74.5 mg, 0.350 mmol, 1.0 equiv.) as the starting materials to give 8-(1-(2-methoxyethyl)-1H-pyrazolo[3,4-b]pyrazin-6-yl)-2-(4-(trifluoromethyl)pyridin-2-yl)-2,8-diazaspiro[4.5]decane (80.7 mg, 49.8%) as a yellow semi-solid. ¹H NMR (400 MHz, DMSO-d₆) δ 8.44 (s, 1H), 8.28 (d, J=5.2 Hz, 1H), 8.04 (s, 1H), 6.79 (dd, J=5.2, 1.5 Hz, 1H), 6.69 (s, 1H), 4.40 (t, J=5.5 Hz, 2H), 3.89 (dt, J=13.7, 5.2 Hz, 2H), 3.82-3.66 (m, 4H), 3.55 (t, J=7.0 Hz, 2H), 3.43 (s, 2H), 3.21 (s, 3H), 1.94 (t, J=7.0 Hz, 2H), 1.76-1.50 (m, 4H). MS m/z: 462.25 [M+H]⁺.

### Example 751: 8-(1-(2-(difluoromethoxy)ethyl)-1H-pyrazolo[3,4-b]pyrazin-6-yl)-2-(4-(trifluoromethyl)pyridin-2-yl)-2,8-diazaspiro[4.5]decane

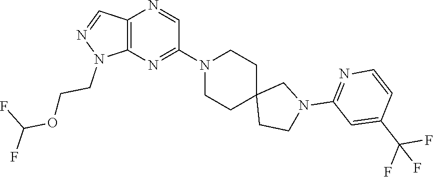 [SMILES: FC(F)OCCn1ncc2ncc(N3CCC4(CCN(c5cc(C(F)(F)F)ccn5)C4)CC3)nc21]

Step 1: 2-(6-chloro-1H-pyrazolo[3,4-b]pyrazin-1-yl)ethan-1-ol:

Followed the general procedure C using 6-chloro-1H-pyrazolo[3,4-b]pyrazine (300 mg, 1.94 mmol, 1.0 equiv.) and ethane-1,2-diol (146 mg, 2.32 mmol, 1.2 equiv.) as the starting materials to give 2-(6-chloro-1H-pyrazolo[3,4-b]pyrazin-1-yl)ethan-1-ol (250 mg, 64.9%) as a light yellow solid. MS m/z: 199 [M+H]⁺.

Step 2: 6-chloro-1-(2-(difluoromethoxy)ethyl)-1H-pyrazolo[3,4-b]pyrazine: A mixture of 2-(6-chloro-1H-pyrazolo[3,4-b]pyrazin-1-yl)ethan-1-ol (250 mg, 1.26 mmol, 1.0 equiv.), 2,2-difluoro-2-(fluorosulfonyl)acetic acid (336 mg, 1.89 mmol, 1.5 equiv.) and CuI (48.0 mg, 0.252 mmol, 0.2 equiv.) in ACN (3 mL) was stirred for 2 h at 50° C. under air atmosphere. Desired product could be detected by LCMS. The resulting mixture was concentrated under reduced pressure. The resulting mixture was extracted with EtOAc (3×20 mL). The combined organic layers were washed with water (2×10 mL), dried over anhydrous Na₂SO₄. After filtration, the filtrate was concentrated under reduced pressure. The residue was purified by silica gel column chromatography, eluted with EtOAc/PE=1/1 to afford 6-chloro-1-(2-(difluoromethoxy)ethyl)-1H-pyrazolo[3,4-b]pyrazine (30.0 mg, 9.59%) as a light yellow solid. MS m/z: 249 [M+H]⁺.

Step 3: 8-(1-(2-(difluoromethoxy)ethyl)-1H-pyrazolo[3,4-b]pyrazin-6-yl)-2-(4-(trifluoromethyl)pyridin-2-yl)-2,8-diazaspiro[4.5]decane: Followed the general procedure I using 6-chloro-1-(2-(difluoromethoxy)ethyl)-1H-pyrazolo[3,4-b]pyrazine (30.0 mg, 0.120 mmol, 1.0 equiv.) and 2-(4-(trifluoromethyl)pyridin-2-yl)-2,8-diazaspiro[4.5]decane hydrochloride (34.5 mg, 0.464 mmol, 1.0 equiv.) as the starting materials to give 8-(1-(2-(difluoromethoxy)ethyl)-1H-pyrazolo[3,4-b]pyrazin-6-yl)-2-(4-(trifluoromethyl)pyridin-2-yl)-2,8-diazaspiro[4.5]decane (4.30 mg, 7.20%) as a yellow solid. ¹H NMR (400 MHz, Methanol-d₄) δ 8.34 (s, 1H), 8.21 (d, J=5.4 Hz, 1H), 7.96 (s, 1H), 6.76 (dd, J=5.4, 1.5 Hz, 1H), 6.70 (s, 1H), 6.29 (t, J=74.8 Hz, 1H), 4.55 (t, J=5.5 Hz, 2H), 4.30 (t, J=5.5 Hz, 2H), 4.01-3.90 (m, 2H), 3.88-3.74 (m, 2H), 3.60 (t, J=7.1 Hz, 2H), 3.48 (s, 2H), 2.05 (t, J=7.1 Hz, 2H), 1.89-1.69 (m, 4H). MS m/z: 498.1 [M+H]⁺.

### Example 752: 8-(1-(1,1-difluoropropan-2-yl)-1H-pyrazolo[3,4-b]pyrazin-6-yl)-2-(4-(trifluoromethyl)pyridin-2-yl)-2,8-diazaspiro[4.5]decane

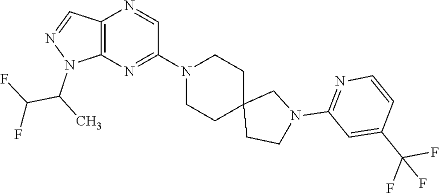 [SMILES: CC(C(F)F)n1ncc2ncc(N3CCC4(CCN(c5cc(C(F)(F)F)ccn5)C4)CC3)nc21]

Step 1: 6-chloro-1-(1,1-difluoropropan-2-yl)-1H-pyrazolo[3,4-b]pyrazine: Followed the general procedure C using 6-chloro-1H-pyrazolo[3,4-b]pyrazine (100 mg, 0.645 mmol, 1.0 equiv.) and 1,1-difluoropropan-2-ol (75.1 mg, 0.774 mmol, 1.2 equiv.) as the starting materials to give 6-chloro-1-(1,1-difluoropropan-2-yl)-1H-pyrazolo[3,4-b]pyrazine (90.0 mg, 59%) as a light yellow solid. MS m/z: 233 [M+H]⁺.

Step 2: 8-(1-(1,1-difluoropropan-2-yl)-1H-pyrazolo[3,4-b]pyrazin-6-yl)-2-(4-(trifluoromethyl)pyridin-2-yl)-2,8-diazaspiro[4.5]decane: Followed the general procedure I using 6-chloro-1-(1,1-difluoropropan-2-yl)-1H-pyrazolo[3,4-b]pyrazine (90.0 mg, 0.386 mmol, 1.0 equiv.) and 2-(4-(trifluoromethyl)pyridin-2-yl)-2,8-diazaspiro[4.5]decane hydrochloride (133 mg, 0.464 mmol, 1.2 equiv.) as the starting materials to give 8-(1-(1,1-difluoropropan-2-yl)-1H-pyrazolo[3,4-b]pyrazin-6-yl)-2-(4-(trifluoromethyl)pyridin-2-yl)-2,8-diazaspiro[4.5]decane (66.5 mg, 35.6%) as a white solid. ¹H NMR (400 MHz, DMSO-d₆) δ 8.48 (s, 1H), 8.28 (d, J=5.2 Hz, 1H), 8.12 (s, 1H), 6.79 (dd, J=5.2, 1.5 Hz, 1H), 6.69 (s, 1H), 6.33 (td, J=55.5, 4.9 Hz, 1H), 5.07 (tt, J=12.0, 6.0 Hz, 1H), 3.90 (d, J=13.6 Hz, 2H), 3.74 (td, J=7.8, 3.8 Hz, 2H), 3.55 (t, J=7.0 Hz, 2H), 3.43 (s, 2H), 1.94 (t, J=7.0 Hz, 2H), 1.67 (q, J=6.8, 6.0 Hz, 4H), 1.59 (d, J=7.1 Hz, 3H). MS m/z: 482.2 [M+H]⁺.

### Example 753: 8-(1-(2,2-difluoroethyl)-1H-pyrazolo[3,4-b]pyrazin-6-yl)-2-(4-methyl-6-(trifluoromethyl)pyridin-3-yl)-2,8-diazaspiro[4.5]decane

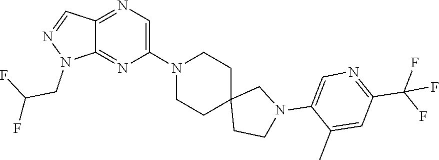 [SMILES: Cc1cc(C(F)(F)F)ncc1N1CCC2(CCN(c3cnc4cnn(CC(F)F)c4n3)CC2)C1]

Step 1: tert-butyl 2-(4-methyl-6-(trifluoromethyl)pyridin-3-yl)-2,8-diazaspiro[4.5]decane-8-carboxylate: Followed the general procedure Y using tert-butyl 2,8-diazaspiro[4.5]decane-8-carboxylate (120 mg, 0.5 mmol, 1 equiv) and 5-chloro-4-methyl-2-(trifluoromethyl)pyridine (117 mg, 0.6 mmol, 1.2 equiv.) as the starting materials, XPhos Pd G3 (43 mg, 0.05 mmol, 0.1 equiv.) as the catalyst to give tert-butyl 2-(4-methyl-6-(trifluoromethyl)pyridin-3-yl)-2,8-diazaspiro[4.5]decane-8-carboxylate (140 mg, 76%) as a white solid. MS m/z: 400 [M+H]⁺.

Step 2: 2-(4-methyl-6-(trifluoromethyl)pyridin-3-yl)-2,8-diazaspiro[4.5]decane hydrochloride: Followed the general procedure B using tert-butyl 2-(4-methyl-6-(trifluoromethyl)pyridin-3-yl)-2,8-diazaspiro[4.5]decane-8-carboxylate (140 mg, 0.35 mmol, 1 equiv) as the starting material to give the crude product 2-(4-methyl-6-(trifluoromethyl)pyridin-3-yl)-2,8-diazaspiro[4.5]decane hydrochloride (100 mg). MS m/z: 300 [M+H]⁺.

Step 3: 8-(1-(2,2-difluoroethyl)-1H-pyrazolo[3,4-b]pyrazin-6-yl)-2-(4-methyl-6-(trifluoromethyl)pyridin-3-yl)-2,8-diazaspiro[4.5]decane: Followed the general procedure I using 1-(2,3-difluorobenzyl)-3-(4-(trifluoromethyl)pyridin-2-yl)-1,3,8-triazaspiro[4.5]decane-2,4-dione hydrochloride (100 mg, 0.334 mmol, 1 equiv) and 6-chloro-1-(2,2-difluoroethyl)-1H-pyrazolo[3,4-b]pyrazine (87 mg, 0.401 mmol, 1.2 equiv) as the starting materials to give 8-(1-(2,2-difluoroethyl)-1H-pyrazolo[3,4-b]pyrazin-6-yl)-2-(4-methyl-6-(trifluoromethyl)pyridin-3-yl)-2,8-diazaspiro[4.5]decane (85 mg, 64%) as a white solid. ¹H NMR (400 MHz, DMSO-d₆) δ 8.50 (s, 1H), 8.13 (s, 1H), 8.06 (s, 1H), 7.49 (s, 1H), 6.44 (tt, J=54.9, 3.8 Hz, 1H), 4.70 (td, J=15.0, 3.8 Hz, 2H), 3.82 (q, J=5.9 Hz, 4H), 3.56 (t, J=6.9 Hz, 2H), 3.45-3.36 (m, 2H), 2.43 (s, 3H), 1.89 (t, J=6.9 Hz, 2H), 1.76-1.61 (m, 4H). MS m/z: 482.4 [M+H]⁺.

### Example 754: 2-(4-chloro-6-(trifluoromethyl)pyridin-3-yl)-8-(1-(2,2-difluoroethyl)-1H-pyrazolo[3,4-b]pyrazin-6-yl)-2,8-diazaspiro[4.5]decane

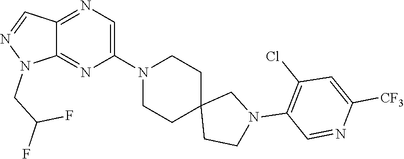 [SMILES: FC(F)Cn1ncc2ncc(N3CCC4(CC3)CCN(c3cnc(C(F)(F)F)cc3Cl)C4)nc21]

Step 1: tert-butyl 2-(4-chloro-6-(trifluoromethyl)pyridin-3-yl)-2,8-diazaspiro[4.5]decane-8-carboxylate: Followed the general procedure V using tert-butyl 2-(6-(trifluoromethyl)pyridin-3-yl)-2,8-diazaspiro[4.5]decane-8-carboxylate (300 mg, 0.778 mmol, 1.0 equiv) as the starting material, NCS (104 mg, 0.778 mmol, 1.0 equiv) as the reagent to give tert-butyl 2-(4-chloro-6-(trifluoromethyl)pyridin-3-yl)-2,8-diazaspiro[4.5]decane-8-carboxylate (35.0 mg, 10.7%) as a white solid. MS m/z: 364 [M-tBu+H]⁺.

Step 2: 2-(4-chloro-6-(trifluoromethyl)pyridin-3-yl)-2,8-diazaspiro[4.5]decane hydrochloride: Followed the general procedure B using tert-butyl 2-(4-chloro-6-(trifluoromethyl)pyridin-3-yl)-2,8-diazaspiro[4.5]decane-8-carboxylate (35.0 mg, 0.083 mmol, 1.00 equiv) as the starting material to give the crude product 2-(4-chloro-6-(trifluoromethyl)pyridin-3-yl)-2,8-diazaspiro[4.5]decane hydrochloride (30.0 mg) (40.0 mg). MS m/z: 320 [M+H]⁺

Step 3: 2-(4-chloro-6-(trifluoromethyl)pyridin-3-yl)-8-(1-(2,2-difluoroethyl)-1H-pyrazolo[3,4-b]pyrazin-6-yl)-2,8-diazaspiro[4.5]decane: Followed the general procedure I using 2-(4-chloro-6-(trifluoromethyl)pyridin-3-yl)-2,8-diazaspiro[4.5]decane hydrochloride (35.0 mg, 0.109 mmol, 1.0 equiv) and 6-chloro-1-(2,2-difluoroethyl)-1H-pyrazolo[3,4-b]pyrazine (28.7 mg, 0.131 mmol, 1.2 equiv) as the starting materials to give 2-(4-chloro-6-(trifluoromethyl)pyridin-3-yl)-8-(1-(2,2-difluoroethyl)-1H-pyrazolo[3,4-b]pyrazin-6-yl)-2,8-diazaspiro[4.5]decane (9.2 mg, 21.2%) as a white solid. ¹H NMR (300 MHz, Methanol-d₄) δ 8.36 (s, 1H), 8.16 (s, 1H), 7.99 (s, 1H), 7.65 (s, 1H), 6.46-6.06 (m, 1H), 4.78-4.58 (m, 2H), 4.18-3.67 (m, 6H), 3.58 (s, 2H), 2.00 (t, 2H), 1.91-1.64 (m, 4H). MS m/z: 502.05 [M+H]⁺.

### Example 755: 8-(1-(2,2-difluoroethyl)-1H-pyrazolo[3,4-b]pyrazin-6-yl)-2-(4,6-dimethylpyridazin-3-yl)-2,8-diazaspiro[4.5]decane

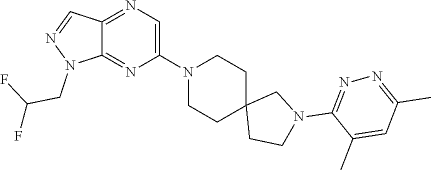 [SMILES: Cc1cc(C)c(N2CCC3(CCN(c4cnc5cnn(CC(F)F)c5n4)CC3)C2)nn1]

Step 1: tert-butyl 2-(4,6-dimethylpyridazin-3-yl)-2,8-diazaspiro[4.5]decane-8-carboxylate: Followed the general procedure Y using tert-butyl 2,8-diazaspiro[4.5]decane-8-carboxylate (200 mg, 0.83 mmol, 1 equiv) and 3-chloro-4,6-dimethylpyridazine (130 mg, 0.91 mmol, 1.1 equiv.) as the starting materials, RuPhos Pd G3 (70 mg, 0.08 mmol, 0.1 equiv.) as the catalyst to give tert-butyl 2-(4,6-dimethylpyridazin-3-yl)-2,8-diazaspiro[4.5]decane-8-carboxylate (150 mg, 52%) as a white solid. MS m/z: 347 [M+H]⁺.

Step 2: 2-(4,6-dimethylpyridazin-3-yl)-2,8-diazaspiro[4.5]decane hydrochloride: Followed the general procedure B using tert-butyl 2-(4,6-dimethylpyridazin-3-yl)-2,8-diazaspiro[4.5]decane-8-carboxylate (150 mg, 0.43 mmol, 1 equiv) as the starting material to give the crude product 2-(4,6-dimethylpyridazin-3-yl)-2,8-diazaspiro[4.5]decane hydrochloride (100 mg). MS m/z: 247 [M+H]⁺.

Step 3: 8-(1-(2,2-difluoroethyl)-1H-pyrazolo[3,4-b]pyrazin-6-yl)-2-(4,6-dimethylpyridazin-3-yl)-2,8-diazaspiro[4.5]decane: Followed the general procedure I using 2-(4,6-dimethylpyridazin-3-yl)-2,8-diazaspiro[4.5]decane hydrochloride (60 mg, 0.243 mmol, 1 equiv) and 6-chloro-1-(2,2-difluoroethyl)-1H-pyrazolo[3,4-b]pyrazine (63 mg, 0.291 mmol, 1.2 equiv) as the starting materials to give 8-(1-(2,2-difluoroethyl)-1H-pyrazolo[3,4-b]pyrazin-6-yl)-2-(4,6-dimethylpyridazin-3-yl)-2,8-diazaspiro[4.5]decane (30 mg, 30%) as a white solid. ¹H NMR (400 MHz, DMSO-d₆) δ 8.48 (s, 1H), 8.16-8.10 (m, 1H), 7.09 (s, 1H), 6.44 (tt, J=54.9, 3.8 Hz, 1H), 4.69 (td, J=15.0, 3.8 Hz, 2H), 3.89-3.74 (m, 4H), 3.65 (t, J=7.0 Hz, 2H), 3.47 (s, 2H), 2.39 (s, 3H), 2.31 (s, 3H), 1.88 (t, J=7.0 Hz, 2H), 1.78-1.60 (m, 4H). MS m/z: 429.15 [M+H]⁺.

### Example 756: 8-(1-(2,2-difluoroethyl)-1H-pyrazolo[3,4-b]pyrazin-6-yl)-2-(4-methyl-6-(trifluoromethyl)pyridazin-3-yl)-2,8-diazaspiro[4.5]decane

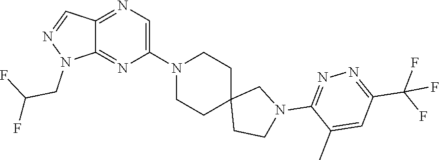 [SMILES: Cc1cc(C(F)(F)F)nnc1N1CCC2(CCN(c3cnc4cnn(CC(F)F)c4n3)CC2)C1]

Step 1: 4-methyl-6-(trifluoromethyl)pyridazin-3-amine: Followed the general procedure L using 4-bromo-6-(trifluoromethyl)pyridazin-3-amine (100 mg, 0.413 mmol, 1.0 equiv.) and methylboronic acid (123 mg, 2.06 mmol, 5.0 equiv.) as the starting materials to give 4-methyl-6-(trifluoromethyl)pyridazin-3-amine (70 mg, 95%) as a white solid. MS m/z: 178 [M+H]⁺.

Step 2: 3-chloro-4-methyl-6-(trifluoromethyl)pyridazine: To a stirred solution of 4-methyl-6-(trifluoromethyl)pyridazin-3-amine (80 mg, 0.452 mmol, 1.0 equiv.) and isopentyl nitrite (105 mg, 0.904 mmol, 2.0 equiv.) in THF (2.0 mL) was added CuCl2 (121 mg, 0.904 mmol, 2.0 equiv.) at 0° C. The resulting mixture was stirred overnight at room temperature. Desired product could be detected by LCMS. The resulting mixture was concentrated under vacuum. The residue was purified with Combi-flash (40 g silico gel column), eluted with gradient of Hex:EtOAc, to afford 3-chloro-4-methyl-6-(trifluoromethyl)pyridazine (60 mg, 67%) as a yellow solid. MS m/z: 197 [M+H]⁺.

Step 3: tert-butyl 2-[4-methyl-6-(trifluoromethyl)pyridazin-3-yl]-2,8-diazaspiro[4.5]decane-8-carboxylate: Followed the general procedure I using 3-chloro-4-methyl-6-(trifluoromethyl)pyridazine (30 mg, 0.153 mmol, 1.0 equiv.) and tert-butyl 2,8-diazaspiro[4.5]decane-8-carboxylate (44 mg, 0.184 mmol, 1.2 equiv.) as the starting materials to give tert-butyl 2-[4-methyl-6-(trifluoromethyl)pyridazin-3-yl]-2,8-diazaspiro[4.5]decane-8-carboxylate (20 mg, 32%) as a white solid. MS m/z: 401 [M+H]⁺.

Step 4: 2-[4-methyl-6-(trifluoromethyl)pyridazin-3-yl]-2,8-diazaspiro[4.5]decane hydrochloride: Followed the general procedure B using tert-butyl 2-[4-methyl-6-(trifluoromethyl)pyridazin-3-yl]-2,8-diazaspiro[4.5]decane-8-carboxylate (55 mg) as the starting material to give the crude product 2-[4-methyl-6-(trifluoromethyl)pyridazin-3-yl]-2,8-diazaspiro[4.5]decane hydrochloride (50 mg). MS m/z: 301 [M+H]⁺.

Step 5: 8-(1-(2,2-difluoroethyl)-1H-pyrazolo[3,4-b]pyrazin-6-yl)-2-(4-methyl-6-(trifluoromethyl)pyridazin-3-yl)-2,8-diazaspiro[4.5]decane: Followed the general procedure I using 2-[4-methyl-6-(trifluoromethyl)pyridazin-3-yl]-2,8-diazaspiro[4.5]decane hydrochloride (55 mg, 0.163 mmol, 1.0 equiv.) and 6-chloro-1-(2,2-difluoroethyl)pyrazolo[3,4-b]pyrazine (35 mg, 0.163 mmol, 1.0 equiv.) as the starting materials to give 8-(1-(2,2-difluoroethyl)-1H-pyrazolo[3,4-b]pyrazin-6-yl)-2-(4-methyl-6-(trifluoromethyl)pyridazin-3-yl)-2,8-diazaspiro[4.5]decane (25 mg, 31%) as a white solid. ¹H NMR (300 MHz, DMSO-d₆) δ 8.50 (s, 1H), 8.13 (s, 1H), 7.61 (s, 1H), 6.44 (tt, 1H), 4.69 (td, 2H), 3.88-3.82 (m, 6H), 3.67 (s, 2H), 1.95-1.91 (m, 2H), 1.75-1.65 (m, 4H). MS m/z: 483.25 [M+H]⁺.

### Example 757: 8-(1-(2,2-difluoroethyl)-1H-pyrazolo[3,4-b]pyrazin-6-yl)-2-(6-methylpyridazin-3-yl)-2,8-diazaspiro[4.5]decane

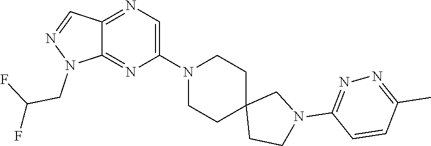 [SMILES: Cc1ccc(N2CCC3(CCN(c4cnc5cnn(CC(F)F)c5n4)CC3)C2)nn1]

Step 1: tert-butyl 2-(6-methylpyridazin-3-yl)-2,8-diazaspiro[4.5]decane-8-carboxylate: Followed the general procedure I using tert-butyl 2,8-diazaspiro[4.5]decane-8-carboxylate (200 mg, 829 μmol, 1.0 equiv.) and 3-chloro-6-methylpyridazine (108 mg, 829 μmol, 1.0 equiv.) as the starting materials to give tert-butyl 2-(6-methylpyridazin-3-yl)-2,8-diazaspiro[4.5]decane-8-carboxylate (210 mg, 75%) as a white solid. MS m/z: 333⁺.

Step 2: 2-(6-methylpyridazin-3-yl)-2,8-diazaspiro[4.5]decane: Followed the general procedure H using tert-butyl 2-(6-methylpyridazin-3-yl)-2,8-diazaspiro[4.5]decane-8-carboxylate (210 mg) as the starting material to give the crude product 2-(6-methylpyridazin-3-yl)-2,8-diazaspiro[4.5]decane (160 mg). MS m/z: 233 [M+H]⁺.

Step 3: 8-(1-(2,2-difluoroethyl)-1H-pyrazolo[3,4-b]pyrazin-6-yl)-2-(6-methylpyridazin-3-yl)-2,8-diazaspiro[4.5]decane: Followed the general procedure I using 2-(6-methylpyridazin-3-yl)-2,8-diazaspiro[4.5]decane (70 mg, 300 μmol, 1.0 equiv.) and 6-chloro-1-(2,2-difluoroethyl)-1H-pyrazolo[3,4-b]pyrazine (66 mg, 300 μmol, 1.0 equiv.) as the starting materials to give 8-(1-(2,2-difluoroethyl)-1H-pyrazolo[3,4-b]pyrazin-6-yl)-2-(6-methylpyridazin-3-yl)-2,8-diazaspiro[4.5]decane (20 mg, 16%) as a white solid. ¹H NMR (400 MHz, DMSO-d₆) δ 8.49 (s, 1H), 8.16 (d, J=17.4 Hz, 1H), 7.23 (d, J=9.1 Hz, 1H), 6.81 (d, J=9.2 Hz, 1H), 6.58-6.31 (m, 1H), 4.70 (td, J=15.0, 3.8 Hz, 2H), 3.89 (dt, J=11.7, 5.2 Hz, 2H), 3.76 (dt, J=12.8, 5.2 Hz, 2H), 3.52 (t, J=7.0 Hz, 4H), 2.41 (s, 3H), 1.95 (t, J=7.0 Hz, 2H), 1.67 (dt, J=8.2, 4.2 Hz, 4H). MS m/z: 415.15 [M+H]⁺.

### Example 758: 8-(1-(2,2-difluoroethyl)-1H-pyrazolo[3,4-b]pyrazin-6-yl)-2-(2-methylpyrimidin-4-yl)-2,8-diazaspiro[4.5]decane

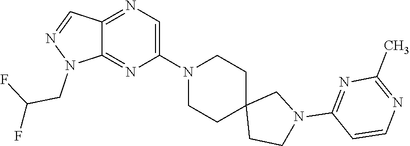 [SMILES: Cc1nccc(N2CCC3(CCN(c4cnc5cnn(CC(F)F)c5n4)CC3)C2)n1]

Step 1: tert-butyl 2-(2-methylpyrimidin-4-yl)-2,8-diazaspiro[4.5]decane-8-carboxylate: Followed the general procedure I using tert-butyl 2,8-diazaspiro[4.5]decane-8-carboxylate (100 mg, 414 μmol. 1.0 equiv.) and 4-chloro-2-methylpyrimidine (103.88 mg, 458 μmol, 1.1 equiv.) as the starting materials to give tert-butyl 2-(2-methylpyrimidin-4-yl)-2,8-diazaspiro[4.5]decane-8-carboxylate (120 mg, 86%) as a yellow oil. MS m/z: 333 [M+H]⁺.

Step 2: 2-(2-methylpyrimidin-4-yl)-2,8-diazaspiro[4.5]decane hydrogen chloride: Followed the general procedure B using tert-butyl 2-(2-methylpyrimidin-4-yl)-2,8-diazaspiro[4.5]decane-8-carboxylate (120 mg) as the starting material to give the crude product 2-(2-methylpyrimidin-4-yl)-2,8-diazaspiro[4.5]decane hydrochloride (120 mg). MS m/z: 233 [M+H]⁺.

Step 3: 8-(1-(2,2-difluoroethyl)-1H-pyrazolo[3,4-b]pyrazin-6-yl)-2-(2-methylpyrimidin-4-yl)-2,8-diazaspiro[4.5]decane: Followed the general procedure I using 6-chloro-1-(2,2-difluoroethyl)pyrazolo[3,4-b]pyrazine (60 mg, 274 μmol, 1.0 equiv.) and 2-(2-methylpyrimidin-4-yl)-2,8-diazaspiro[4.5]decane hydrochloride (100 mg, 301 μmol, 1.1 equiv.) as the starting materials to give 8-[1-(2,2-difluoroethyl)pyrazolo[3,4-b]pyrazin-6-yl]-2-(2-methylpyrimidin-4-yl)-2,8-diazaspiro[4.5]decane (38.6 mg, 33%) as a white solid. ¹H NMR (400 MHz, DMSO-d₆) δ 8.48 (s, 1H), 8.14 (d, J=5.2 Hz, 2H), 8.04 (d, J=6.0 Hz, 1H), 6.61-6.23 (m, 2H), 4.69 (td, J=15.0, 3.8 Hz, 2H), 3.80 (dd, J=12.8, 6.5 Hz, 4H), 3.56 (s, 1H), 3.36-3.29 (m, 2H), 2.35 (s, 3H), 1.93 (s, 2H), 1.65 (q, J=5.4 Hz, 4H). MS m/z: 415.10 [M+H]⁺.

### Example 759: 8-(1-(2,2-difluoroethyl)-1H-pyrazolo[3,4-b]pyrazin-6-yl)-2-((4-(trifluoromethyl)pyridin-3-yl)oxy)-8-azaspiro[4.5]decane

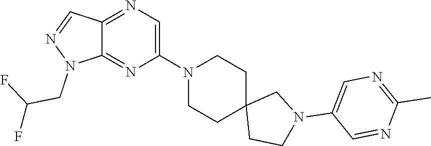 [SMILES: Cc1ncc(N2CCC3(CCN(c4cnc5cnn(CC(F)F)c5n4)CC3)C2)cn1]

Step 1: tert-butyl 2-(2-methylpyrimidin-5-yl)-2,8-diazaspiro[4.5]decane-8-carboxylate: Followed the general procedure Y using tert-butyl 2,8-diazaspiro[4.5]decane-8-carboxylate (120 mg, 500 μmol, 1.0 equiv.) and 5-bromo-2-methylpyrimidine (86 mg, 500 μmol, 1.0 equiv.) as the starting materials, Xphos Pd G3 (42.3 mg, 50 μmol, 0.1 equiv.) as the catalyst to give tert-butyl 2-(2-methylpyrimidin-5-yl)-2,8-diazaspiro[4.5]decane-8-carboxylate (80 mg, 48%) as a yellow oil. MS m/z: 333 [M+H]⁺.

Step 2: 2-(2-methylpyrimidin-5-yl)-2,8-diazaspiro[4.5]decane hydrochloride: Followed the general procedure B using tert-butyl 2-(2-methylpyrimidin-5-yl)-2,8-diazaspiro[4.5]decane-8-carboxylate (80 mg) as the starting material to give the crude product 2-(2-methylpyrimidin-5-yl)-2,8-diazaspiro[4.5]decane hydrochloride (60 mg) as a yellow solid. MS m/z: 233 [M+H]⁺.

Step 3: 8-(1-(2,2-difluoroethyl)-1H-pyrazolo[3,4-b]pyrazin-6-yl)-2-(2-methylpyrimidin-5-yl)-2,8-diazaspiro[4.5]decane: Followed the general procedure I using 2-(2-methylpyrimidin-5-yl)-2,8-diazaspiro[4.5]decane hydrochloride (60 mg, 259 μmol, 1.0 equiv.) and 6-chloro-1-(2,2-difluoroethyl)-1H-pyrazolo[3,4-b]pyrazine (67.7 mg, 310 μmol, 1.2 equiv.) as the starting materials to give 8-(1-(2,2-difluoroethyl)-1H-pyrazolo[3,4-b]pyrazin-6-yl)-2-(2-methylpyrimidin-5-yl)-2,8-diazaspiro[4.5]decane (28.8 mg, 27%) as a white solid. ¹H NMR (400 MHz, DMSO-d₆) δ 8.49 (s, 1H), 8.13 (s, 1H), 8.03 (s, 2H), 6.44 (tt, J=55.0, 3.8 Hz, 1H), 4.69 (td, J=15.0, 3.8 Hz, 2H), 3.94-3.85 (m, 2H), 3.80-3.71 (m, 2H), 3.39-3.34 (m, 2H), 3.24 (s, 2H), 2.46 (s, 3H), 1.93 (t, J=7.0 Hz, 2H), 1.71-1.60 (m, 4H). MS m/z: 415.1 [M+H]⁺.

### Example 760: 8-(1-(2,2-difluoroethyl)-1H-pyrazolo[3,4-b]pyrazin-6-yl)-2-(2-ethylpyrimidin-5-yl)-2,8-diazaspiro[4.5]decane

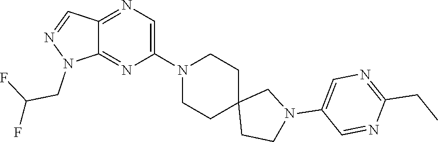 [SMILES: CCc1ncc(N2CCC3(CCN(c4cnc5cnn(CC(F)F)c5n4)CC3)C2)cn1]

Step 1: tert-butyl 2-(2-ethylpyrimidin-5-yl)-2,8-diazaspiro[4.5]decane-8-carboxylate: Followed the general procedure I using tert-butyl 2,8-diazaspiro[4.5]decane-8-carboxylate (100 mg, 0.416 mmol, 1 equiv.) and 5-bromo-2-ethylpyrimidine (78 mg, 0.416 mmol, 1.0 equiv.) as the starting materials to give tert-butyl 2-(2-ethylpyrimidin-5-yl)-2,8-diazaspiro[4.5]decane-8-carboxylate (65 mg, 45%) as a white solid. MS m/z: 347 [M+H]⁺.

Step 2: 2-(2-ethylpyrimidin-5-yl)-2,8-diazaspiro[4.5]decane hydrochloride: Followed the general procedure B using tert-butyl 2-(2-ethylpyrimidin-5-yl)-2,8-diazaspiro[4.5]decane-8-carboxylate (65 mg) as the starting material to give the crude product 2-(2-ethylpyrimidin-5-yl)-2,8-diazaspiro[4.5]decane hydrochloride (50 mg). MS m/z: 247 [M+H]⁺.

Step 3: 8-(1-(2,2-difluoroethyl)-1H-pyrazolo[3,4-b]pyrazin-6-yl)-2-(2-ethylpyrimidin-5-yl)-2,8-diazaspiro[4.5]decane: Followed the general procedure I using 2-(2-ethylpyrimidin-5-yl)-2,8-diazaspiro[4.5]decane hydrochloride (50 mg, 0.203 mmol, 1 equiv.) and 6-chloro-1-(2,2-difluoroethyl)-1H-pyrazolo[3,4-b]pyrazine (45 mg, 0.203 mmol, 1 equiv.) as the starting materials to give 8-(1-(2,2-difluoroethyl)-1H-pyrazolo[3,4-b]pyrazin-6-yl)-2-(2-ethylpyrimidin-5-yl)-2,8-diazaspiro[4.5]decane (48.6 mg, 56%) as a light yellow solid. ¹H NMR (400 MHz, DMSO-d₆) δ 8.49 (s, 1H), 8.13 (s, 1H), 8.05 (s, 2H), 6.44 (tt, J=54.9, 3.8 Hz, 1H), 4.76-4.63 (m, 2H), 3.89 (dt, J=11.4, 5.2 Hz, 2H), 3.81-3.70 (m, 2H), 3.37 (t, J=6.9 Hz, 2H), 3.20 (s, 2H), 2.79-2.69 (m, 2H), 1.94 (t, J=7.0 Hz, 2H), 1.72-1.60 (m, 4H), 1.21 (t, J=7.6 Hz, 3H). MS m/z: 429.2 [M+H]⁺.

### Example 761: 8-(1-(2,2-difluoroethyl)-1H-pyrazolo[3,4-b]pyrazin-6-yl)-2-(4-methyl-2-(trifluoromethyl)pyrimidin-5-yl)-2,8-diazaspiro[4.5]decane

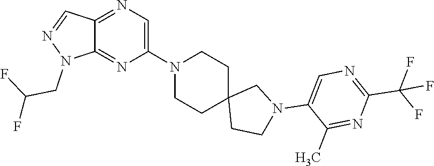 [SMILES: Cc1nc(C(F)(F)F)ncc1N1CCC2(CCN(c3cnc4cnn(CC(F)F)c4n3)CC2)C1]

Step 1: tert-butyl 2-(4-methyl-2-(trifluoromethyl)pyrimidin-5-yl)-2,8-diazaspiro[4.5]decane-8-carboxylate: Followed the general procedure L using tert-butyl 2-(4-bromo-2-(trifluoromethyl)pyrimidin-5-yl)-2,8-diazaspiro[4.5]decane-8-carboxylate (100 mg, 0.215 mmol, 1.0 equiv.) and methylboronic acid (64.4 mg, 1.07 mmol, 5.0 equiv.) as the starting materials to give tert-butyl 2-(4-methyl-2-(trifluoromethyl)pyrimidin-5-yl)-2,8-diazaspiro[4.5]decane-8-carboxylate (60.0 mg, 69.7%) as a light yellow solid. MS m/z: 401 [M+H]⁺.

Step 2: 2-(4-methyl-2-(trifluoromethyl)pyrimidin-5-yl)-2,8-diazaspiro[4.5]decane hydrochloride: Followed the general procedure B using tert-butyl 2-(4-methyl-2-(trifluoromethyl)pyrimidin-5-yl)-2,8-diazaspiro[4.5]decane-8-carboxylate (60.0 mg, 0.150 mmol, 1.0 equiv.) as the starting materials to give the crude product 2-(4-methyl-2-(trifluoromethyl)pyrimidin-5-yl)-2,8-diazaspiro[4.5]decane hydrochloride (50.0 mg) as a light yellow solid. MS m/z: 301 [M+H]⁺

Step 3: 8-(1-(2,2-difluoroethyl)-1H-pyrazolo[3,4-b]pyrazin-6-yl)-2-(4-methyl-2-(trifluoromethyl)pyrimidin-5-yl)-2,8-diazaspiro[4.5]decane: Followed the general procedure I using 2-(4-methyl-2-(trifluoromethyl)pyrimidin-5-yl)-2,8-diazaspiro[4.5]decane hydrochloride (50.0 mg, 0.166 mmol, 1.0 equiv.) and 6-chloro-1-(2,2-difluoroethyl)-1H-pyrazolo[3,4-b]pyrazine (43.7 mg, 0.299 mmol, 1.2 equiv.) as the starting materials to give 8-(1-(2,2-difluoroethyl)-1H-pyrazolo[3,4-b]pyrazin-6-yl)-2-(4-methyl-2-(trifluoromethyl)pyrimidin-5-yl)-2,8-diazaspiro[4.5]decane (20.0 mg, 24.9%) as a white solid. ¹H NMR (400 MHz, DMSO-d₆) δ 8.50 (s, 1H), 8.14 (d, J=5.1 Hz, 2H), 6.44 (tt, J=55.0, 3.8 Hz, 1H), 4.69 (td, J=15.1, 3.8 Hz, 2H), 3.94-3.74 (m, 4H), 3.60 (t, J=6.9 Hz, 2H), 3.42 (s, 2H), 2.65 (s, 3H), 1.91 (t, J=6.9 Hz, 2H), 1.76-1.60 (m, 4H). MS m/z: 483.25 [M+H]⁺.

### Example 762: 8-(1-(2,2-difluoroethyl)-1H-pyrazolo[3,4-b]pyrazin-6-yl)-2-(2-methoxypyrimidin-5-yl)-2,8-diazaspiro[4.5]decane

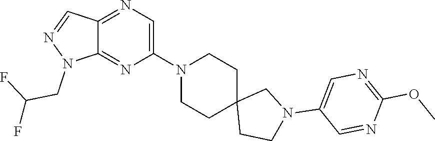 [SMILES: COc1ncc(N2CCC3(CCN(c4cnc5cnn(CC(F)F)c5n4)CC3)C2)cn1]

Step 1: tert-butyl 2-(2-methoxypyrimidin-5-yl)-2,8-diazaspiro[4.5]decane-8-carboxylate: Followed the general procedure Y using tert-butyl 2,8-diazaspiro[4.5]decane-8-carboxylate (120 mg, 500 μmol, 1.0 equiv.) and 5-iodo-2-methoxypyrimidine (118 mg, 500 μmol, 1.0 equiv.) as the starting materials, Xphos Pd G3 (42.3 mg, 50 μmol, 0.1 equiv.) as the catalyst to give tert-butyl 2-(2-methoxypyrimidin-5-yl)-2,8-diazaspiro[4.5]decane-8-carboxylate (80 mg, 46%) as a yellow oil. MS m/z: 349 [M+H]⁺.

Step 2: 2-(2-methoxypyrimidin-5-yl)-2,8-diazaspiro[4.5]decane hydrochloride: Followed the general procedure B using tert-butyl 2-(2-methoxypyrimidin-5-yl)-2,8-diazaspiro[4.5]decane-8-carboxylate (80 mg) as the starting material to give the crude product 2-(2-methoxypyrimidin-5-yl)-2,8-diazaspiro[4.5]decane hydrochloride (60 mg) as a yellow solid. MS m/z: 249 [M+H]⁺.

Step 3: 8-(1-(2,2-difluoroethyl)-1H-pyrazolo[3,4-b]pyrazin-6-yl)-2-(2-methoxypyrimidin-5-yl)-2,8-diazaspiro[4.5]decane: Followed the general procedure I using 2-(2-methoxypyrimidin-5-yl)-2,8-diazaspiro[4.5]decane hydrochloride (60 mg, 242 μmol, 1.0 equiv.) and 6-chloro-1-(2,2-difluoroethyl)-1H-pyrazolo[3,4-b]pyrazine (63.3 mg, 290 μmol, 1.2 equiv.) as the starting materials to give 8-(1-(2,2-difluoroethyl)-1H-pyrazolo[3,4-b]pyrazin-6-yl)-2-(2-methoxypyrimidin-5-yl)-2,8-diazaspiro[4.5]decane (7.2 mg, 6.9%) as a white solid. ¹H NMR (400 MHz, DMSO-d₆) δ 8.49 (s, 1H), 8.13 (s, 1H), 7.98 (s, 2H), 6.41 (tt, 1H), 4.69 (td, J=15.0, 3.8 Hz, 2H), 3.92-3.83 (m, 2H), 3.81 (s, 3H), 3.79-3.71 (m, 2H), 3.40-3.34 (m, 2H), 3.20 (s, 2H), 1.93 (t, J=6.9 Hz, 2H), 1.68-1.63 (m, 4H). MS m/z: 431.1 [M+H]⁺.

### Example 763: 8-(1-(2,2-difluoroethyl)-1H-pyrazolo[3,4-b]pyrazin-6-yl)-2-(2-ethoxypyrimidin-5-yl)-2,8-diazaspiro[4.5]decane

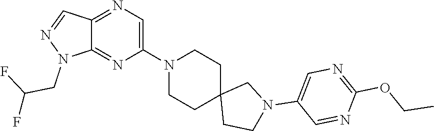 [SMILES: CCOc1ncc(N2CCC3(CCN(c4cnc5cnn(CC(F)F)c5n4)CC3)C2)cn1]

Step 1: tert-butyl 2-(2-ethoxypyrimidin-5-yl)-2,8-diazaspiro[4.5]decane-8-carboxylate: Followed the general procedure Y using tert-butyl 2,8-diazaspiro[4.5]decane-8-carboxylate (120 mg, 500 μmol, 1.0 equiv.) and 5-bromo-2-ethoxypyrimidine (101 mg, 500 μmol, 1.0 equiv.) as the starting materials, Xphos Pd G3 (42.3 mg, 50 μmol, 0.1 equiv.) as the catalyst to give tert-butyl 2-(2-ethoxypyrimidin-5-yl)-2,8-diazaspiro[4.5]decane-8-carboxylate (80 mg, 44%) as a yellow oil. MS m/z: 363 [M+H]⁺.

Step 2: 2-(2-ethoxypyrimidin-5-yl)-2,8-diazaspiro[4.5]decane hydrochloride: Followed the general procedure B using tert-butyl 2-(2-ethoxypyrimidin-5-yl)-2,8-diazaspiro[4.5]decane-8-carboxylate (80 mg) as the starting material to give the crude product 2-(2-ethoxypyrimidin-5-yl)-2,8-diazaspiro[4.5]decane hydrochloride (70 mg) as a yellow solid. MS m/z: 263 [M+H]⁺.

Step 3: 8-(1-(2,2-difluoroethyl)-1H-pyrazolo[3,4-b]pyrazin-6-yl)-2-(2-ethoxypyrimidin-5-yl)-2,8-diazaspiro[4.5]decane: Followed the general procedure I using 2-(2-ethoxypyrimidin-5-yl)-2,8-diazaspiro[4.5]decane hydrochloride (70 mg, 266 μmol, 1.0 equiv.) and 6-chloro-1-(2,2-difluoroethyl)-1H-pyrazolo[3,4-b]pyrazine (69.6 mg, 319 μmol, 1.2 equiv.) as the starting materials to give 8-(1-(2,2-difluoroethyl)-1H-pyrazolo[3,4-b]pyrazin-6-yl)-2-(2-ethoxypyrimidin-5-yl)-2,8-diazaspiro[4.5]decane (27.2 mg, 22%) as a white solid. ¹H NMR (400 MHz, DMSO-d₆) δ 8.49 (s, 1H), 8.14 (s, 1H), 7.97 (s, 2H), 6.44 (tt, J=54.9, 3.8 Hz, 1H), 4.70 (td, J=15.0, 3.8 Hz, 2H), 4.22 (q, J=7.0 Hz, 2H), 3.94-3.83 (m, 2H), 3.80-3.70 (m, 2H), 3.33-3.32 (m, 1H), 3.32-3.29 (m, 1H), 3.20 (s, 2H), 1.92 (t, J=6.9 Hz, 2H), 1.71-1.60 (m, 4H), 1.29 (t, J=7.0 Hz, 3H). MS m/z: 445.2 [M+H]⁺.

### Example 764: 8-(1-(2,2-difluoroethyl)-1H-pyrazolo[3,4-b]pyrazin-6-yl)-2-(5-methyl-6-(trifluoromethyl)pyrazin-2-yl)-2,8-diazaspiro[4.5]decane

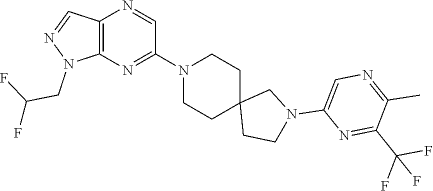 [SMILES: Cc1ncc(N2CCC3(CCN(c4cnc5cnn(CC(F)F)c5n4)CC3)C2)nc1C(F)(F)F]

Step 1: tert-butyl 2-(5-chloro-6-(trifluoromethyl)pyrazin-2-yl)-2,8-diazaspiro[4.5]decane-8-carboxylate: Followed the general procedure V using tert-butyl 2-(6-(trifluoromethyl)pyrazin-2-yl)-2,8-diazaspiro[4.5]decane-8-carboxylate (200 mg, 0.517 mmol, 1.0 equiv.) as the starting material, NCS (88.1 mg, 0.620 mmol, 1.2 equiv.) as the reagent to give tert-butyl 2-(5-chloro-6-(trifluoromethyl)pyrazin-2-yl)-2,8-diazaspiro[4.5]decane-8-carboxylate (170 mg, 78.1%) as a white solid. MS m/z: 421 [M+H]⁺.

Step 2: tert-butyl 2-(5-methyl-6-(trifluoromethyl)pyrazin-2-yl)-2,8-diazaspiro[4.5]decane-8-carboxylate: Followed the general procedure L using tert-butyl 2-(5-chloro-6-(trifluoromethyl)pyrazin-2-yl)-2,8-diazaspiro[4.5]decane-8-carboxylate (170 mg, 0.404 mmol, 1.0 equiv.) and methylboronic acid (242 mg, 4.04 mmol, 10.0 equiv.) as the starting materials to give tert-butyl 2-(5-methyl-6-(trifluoromethyl)pyrazin-2-yl)-2,8-diazaspiro[4.5]decane-8-carboxylate (100 mg, 61.8%) as a light yellow solid. MS m/z: 401 [M+H]⁺.

Step 3: 2-(5-methyl-6-(trifluoromethyl)pyrazin-2-yl)-2,8-diazaspiro[4.5]decane hydrochloride: Followed the general procedure B using tert-butyl 2-(5-methyl-6-(trifluoromethyl)pyrazin-2-yl)-2,8-diazaspiro[4.5]decane-8-carboxylate (100 mg, 0.249 mmol, 1.0 equiv.) as the starting materials to give the crude product 2-(5-methyl-6-(trifluoromethyl)pyrazin-2-yl)-2,8-diazaspiro[4.5]decane hydrochloride (90.0 mg) as a light yellow solid. MS m/z: 301 [M+H]⁺

Step 4: 8-(1-(2,2-difluoroethyl)-1H-pyrazolo[3,4-b]pyrazin-6-yl)-2-(5-methyl-6-(trifluoromethyl)pyrazin-2-yl)-2,8-diazaspiro[4.5]decane: Followed the general procedure I using 2-(5-methyl-6-(trifluoromethyl)pyrazin-2-yl)-2,8-diazaspiro[4.5]decane hydrochloride (90.0 mg, 0.299 mmol, 1.0 equiv.) and 6-chloro-1-(2,2-difluoroethyl)-1H-pyrazolo[3,4-b]pyrazine (65.5 mg, 0.299 mmol, 1.0 equiv.) as the starting materials to give 8-(1-(2,2-difluoroethyl)-1H-pyrazolo[3,4-b]pyrazin-6-yl)-2-(5-methyl-6-(trifluoromethyl)pyrazin-2-yl)-2,8-diazaspiro[4.5]decane (13.4 mg, 16.9%) as a white solid. ¹H NMR (400 MHz, DMSO-d₆) δ 8.49 (s, 1H), 8.15 (d, J=13.6 Hz, 2H), 6.44 (tt, J=54.9, 3.8 Hz, 1H), 4.69 (td, J=15.0, 3.8 Hz, 2H), 3.81 (q, J=7.5, 5.2 Hz, 4H), 3.57 (t, J=7.0 Hz, 2H), 3.42 (s, 2H), 2.46 (t, J=2.3 Hz, 3H), 1.96 (t, J=7.0 Hz, 2H), 1.68 (q, J=6.4 Hz, 4H). MS m/z: 483.2 [M+H]⁺.

### Example 765: 8-(1-(2,2-difluoroethyl)-1H-pyrazolo[3,4-b]pyrazin-6-yl)-2-(3-methyl-6-(trifluoromethyl)pyrazin-2-yl)-2,8-diazaspiro[4.5]decane

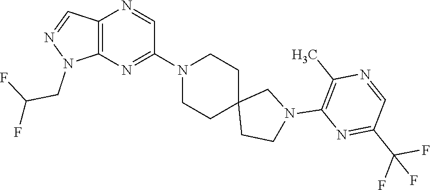 [SMILES: Cc1ncc(C(F)(F)F)nc1N1CCC2(CCN(c3cnc4cnn(CC(F)F)c4n3)CC2)C1]

Step 1: tert-butyl 2-(3,5-dichloro-6-(trifluoromethyl)pyrazin-2-yl)-2,8-diazaspiro[4.5]decane-8-carboxylate: Followed the general procedure V using tert-butyl 2-(6-(trifluoromethyl)pyrazin-2-yl)-2,8-diazaspiro[4.5]decane-8-carboxylate (300 mg, 0.776 mmol, 1.0 equiv) as the starting material, NCS (100 mg, 414 μmol, 1.0 equiv.) as the reagent to give tert-butyl 2-(3,5-dichloro-6-(trifluoromethyl)pyrazin-2-yl)-2,8-diazaspiro[4.5]decane-8-carboxylate (300 mg, 84.9%) as a white solid. MS m/z: 400 [M−tBu+H]⁺.

Step 2: tert-butyl 2-(5-chloro-3-methyl-6-(trifluoromethyl)pyrazin-2-yl)-2,8-diazaspiro[4.5]decane-8-carboxylate: Followed the general procedure L using tert-butyl 2-(3,5-dichloro-6-(trifluoromethyl)pyrazin-2-yl)-2,8-diazaspiro[4.5]decane-8-carboxylate (300 mg, 0.659 mmol, 1.00 equiv) and methylboronic acid (39.4 mg, 0.659 mmol, 1.0 equiv) as the starting materials to give tert-butyl 2-(5-chloro-3-methyl-6-(trifluoromethyl)pyrazin-2-yl)-2,8-diazaspiro[4.5]decane-8-carboxylate (150 mg, 52.4%) as a white solid. MS m/z: 379 [M−tBu+H]⁺.

Step 3: tert-butyl 2-(3-methyl-6-(trifluoromethyl)pyrazin-2-yl)-2,8-diazaspiro[4.5]decane-8-carboxylate: Followed the general procedure U using tert-butyl 2-(5-chloro-3-methyl-6-(trifluoromethyl)pyrazin-2-yl)-2,8-diazaspiro[4.5]decane-8-carboxylate (150 mg, 0.345 mmol, 1.0 equiv.) as the starting material, Pd(OH)₂/C (15 mg, 20% Pd on carbon, wetted with ca. 50% H₂O) as catalyst to give tert-butyl 2-(3-methyl-6-(trifluoromethyl)pyrazin-2-yl)-2,8-diazaspiro[4.5]decane-8-carboxylate (50.0 mg, 60.4%) as a light yellow oil. MS m/z: 345 [M−tBu+H]+.

Step 4: 2-(3-methyl-6-(trifluoromethyl)pyrazin-2-yl)-2,8-diazaspiro[4.5]decane hydrochloride: Followed the general procedure B using tert-butyl 2-(3-methyl-6-(trifluoromethyl)pyrazin-2-yl)-2,8-diazaspiro[4.5]decane-8-carboxylate (50.0 mg, 0.125 mmol, 1.0 equiv) as the starting material to give the crude product 2-(3-methyl-6-(trifluoromethyl)pyrazin-2-yl)-2,8-diazaspiro[4.5]decane hydrochloride (50.0 mg). MS m/z: 301 [M+H]⁺.

Step 5: 8-(1-(2,2-difluoroethyl)-1H-pyrazolo[3,4-b]pyrazin-6-yl)-2-(3-methyl-6-(trifluoromethyl)pyrazin-2-yl)-2,8-diazaspiro[4.5]decane: Followed the general procedure I using 2-(3-methyl-6-(trifluoromethyl)pyrazin-2-yl)-2,8-diazaspiro[4.5]decane hydrochloride (50.0 mg, 0.148 mmol, 1.0 equiv) and 6-chloro-1-(2,2-difluoroethyl)pyrazolo[3,4-b]pyrazine (38.9 mg, 0.178 mmol, 1.2 equiv) as the starting materials to give 8-(1-(2,2-difluoroethyl)-1H-pyrazolo[3,4-b]pyrazin-6-yl)-2-(3-methyl-6-(trifluoromethyl)pyrazin-2-yl)-2,8-diazaspiro[4.5]decane (16.6 mg, 23.2%) as a white solid. ¹H NMR (300 MHz, Methanol-d₄) δ 8.38 (s, 1H), 8.02 (d, 2H), 6.28 (tt, 1H), 4.75-4.64 (m, 2H), 3.93-3.82 (m, 6H), 3.65 (s, 2H), 2.72 (d, 3H), 2.02 (t, 2H), 1.84-1.78 (m, 4H). MS m/z: 483.1 [M+H]⁺.

### Example 766: 8-(1-(2,2-difluoroethyl)-1H-pyrazolo[3,4-b]pyrazin-6-yl)-2-(3-fluoro-6-(trifluoromethyl)pyrazin-2-yl)-2,8-diazaspiro[4.5]decane

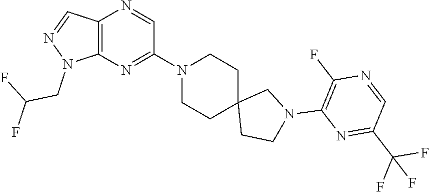 [SMILES: Fc1ncc(C(F)(F)F)nc1N1CCC2(CCN(c3cnc4cnn(CC(F)F)c4n3)CC2)C1]

Step 1: tert-butyl 2-(6-(trifluoromethyl)pyrazin-2-yl)-2,8-diazaspiro[4.5]decane-8-carboxylate: Followed the general procedure I using tert-butyl 2,8-diazaspiro[4.5]decane-8-carboxylate (1.2 g, 5 mmol, 1.0 equiv.) and 2-chloro-6-(trifluoromethyl)pyrazine (0.91 g, 5 mmol, 1.0 equiv.) as the starting materials to give tert-butyl 2-(6-(trifluoromethyl)pyrazin-2-yl)-2,8-diazaspiro[4.5]decane-8-carboxylate (1.1 g, 52%) as a white solid. MS m/z: 387 [M+H]⁺.

Step 2: tert-butyl 2-(3-fluoro-6-(trifluoromethyl)pyrazin-2-yl)-2,8-diazaspiro[4.5]decane-8-carboxylate: A solution of tert-butyl 2-(6-(trifluoromethyl)pyrazin-2-yl)-2,8-diazaspiro[4.5]decane-8-carboxylate (500 mg, 1.29 mmol, 1.0 equiv.) and Selectfluor (688 mg, 1.94 mmol, 1.5 equiv.) in ACN (10 mL) was stirred for 2 h at 80° C. The resulting mixture was concentrated under vacuum. The residue was purified by silica gel column chromatography, eluted with PE/EA (1:1) to afford crude product. The crude product (200 mg) was purified by Prep-HPLC to afford tert-butyl 2-(3-fluoro-6-(trifluoromethyl)pyrazin-2-yl)-2,8-diazaspiro[4.5]decane-8-carboxylate (80 mg, 15%) as a colorless oil. MS m/z: 405 [M+H]⁺.

Step 3: 2-(3-fluoro-6-(trifluoromethyl)pyrazin-2-yl)-2,8-diazaspiro[4.5]decane hydrochloride: Followed the general procedure B using tert-butyl 2-(3-fluoro-6-(trifluoromethyl)pyrazin-2-yl)-2,8-diazaspiro[4.5]decane-8-carboxylate (80 mg) as the starting material to give the crude product 2-(3-fluoro-6-(trifluoromethyl)pyrazin-2-yl)-2,8-diazaspiro[4.5]decane hydrochloride (45 mg). MS m/z: 305 [M+H]⁺.

Step 4: 8-(1-(2,2-difluoroethyl)-1H-pyrazolo[3,4-b]pyrazin-6-yl)-2-(3-fluoro-6-(trifluoromethyl)pyrazin-2-yl)-2,8-diazaspiro[4.5]decane: Followed the general procedure I using 2-(3-fluoro-6-(trifluoromethyl)pyrazin-2-yl)-2,8-diazaspiro[4.5]decane hydrochloride (45 mg, 0.15 mmol, 1.0 equiv.) and 6-chloro-1-(2,2-difluoroethyl)-1H-pyrazolo[3,4-b]pyrazine (35 mg, 0.15 mmol, 1.1 equiv.) as the starting materials to give 8-(1-(2,2-difluoroethyl)-1H-pyrazolo[3,4-b]pyrazin-6-yl)-2-(3-fluoro-6-(trifluoromethyl)pyrazin-2-yl)-2,8-diazaspiro[4.5]decane (5 mg, 6%) as a white solid. ¹H NMR (300 MHz, Methanol-d₄) δ 8.38 (s, 1H), 8.01 (s, 1H), 7.66 (d, J=2.7 Hz, 1H), 6.28 (tt, J=55.5, 4.1 Hz, 1H), 4.69 (td, J=14.1, 4.1 Hz, 2H), 3.96-3.84 (m, 6H), 3.70 (d, J=2.0 Hz, 2H), 2.05 (t, J=7.2 Hz, 2H), 1.86-1.75 (m, 4H). MS m/z: 487.10 [M+H]⁺.

### Example 767: 8-(1-(2,2-difluoroethyl)-1H-pyrazolo[3,4-b]pyrazin-6-yl)-2-(5-fluoro-6-(trifluoromethyl)pyrazin-2-yl)-2,8-diazaspiro[4.5]decane

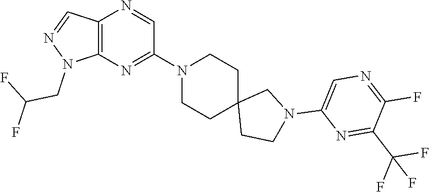 [SMILES: Fc1ncc(N2CCC3(CCN(c4cnc5cnn(CC(F)F)c5n4)CC3)C2)nc1C(F)(F)F]

Step 1: 2-(5-fluoro-6-(trifluoromethyl)pyrazin-2-yl)-2,8-diazaspiro[4.5]decane hydrochloride: Followed the general procedure B using tert-butyl 2-(5-fluoro-6-(trifluoromethyl)pyrazin-2-yl)-2,8-diazaspiro[4.5]decane-8-carboxylate (150 mg) as the starting material to give the crude product 2-(5-fluoro-6-(trifluoromethyl)pyrazin-2-yl)-2,8-diazaspiro[4.5]decane hydrochloride (120 mg) as a yellow solid. MS m/z: 305 [M+H]⁺.

Step 2: 8-(1-(2,2-difluoroethyl)-1H-pyrazolo[3,4-b]pyrazin-6-yl)-2-(5-fluoro-6-(trifluoromethyl)pyrazin-2-yl)-2,8-diazaspiro[4.5]decane: Followed the general procedure I using 2-(5-fluoro-6-(trifluoromethyl)pyrazin-2-yl)-2,8-diazaspiro[4.5]decane (110 mg, 362 μmol, 1.0 equiv.) and 6-chloro-1-(2,2-difluoroethyl)-1H-pyrazolo[3,4-b]pyrazine (78.9 mg, 362 μmol, 1.2 equiv.) as the starting materials to give 8-(1-(2,2-difluoroethyl)-1H-pyrazolo[3,4-b]pyrazin-6-yl)-2-(5-fluoro-6-(trifluoromethyl)pyrazin-2-yl)-2,8-diazaspiro[4.5]decane (88.9 mg, 50%) as an off-white solid. ¹H NMR (300 MHz, DMSO-d₆) δ 8.49 (s, 1H), 8.13 (s, 1H), 8.00-7.86 (m, 1H), 6.71-6.15 (m, 1H), 4.81-4.59 (m, 2H), 3.96-3.70 (m, 4H), 3.58 (t, J=7.0 Hz, 2H), 3.44 (s, 2H), 1.97 (t, J=7.0 Hz, 2H), 1.76-1.58 (m, 4H). MS m/z: 487.15 [M+H]⁺.

### Example 768: 8-(1-(oxetan-3-yl)-1H-pyrazolo[3,4-b]pyrazin-6-yl)-2-(6-(trifluoromethyl)pyrazin-2-yl)-2,8-diazaspiro[4.5]decane

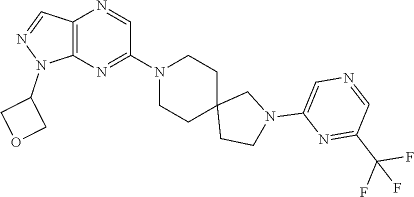 [SMILES: FC(F)(F)c1cncc(N2CCC3(CCN(c4cnc5cnn(C6COC6)c5n4)CC3)C2)n1]

Step 1: tert-butyl 2-(6-(trifluoromethyl)pyrazin-2-yl)-2,8-diazaspiro[4.5]decane-8-carboxylate: Followed the general procedure I using tert-butyl 2,8-diazaspiro[4.5]decane-8-carboxylate (100 mg, 410 μmol, 1.0 equiv.) and 2-chloro-6-(trifluoromethyl)pyrazine (91 mg, 490 μmol, 1.2 equiv.) as the starting materials to give tert-butyl 2-(6-(trifluoromethyl)pyrazin-2-yl)-2,8-diazaspiro[4.5]decane-8-carboxylate (90 mg, 24%) as a white solid. MS m/z: 387 [M+H]⁺.

Step 2: 2-(6-(trifluoromethyl)pyrazin-2-yl)-2,8-diazaspiro[4.5]decane hydrochloride: Followed the general procedure B using tert-butyl 2-(6-(trifluoromethyl)pyrazin-2-yl)-2,8-diazaspiro[4.5]decane-8-carboxylate (90 mg, 230 μmol, 1.0 equiv.) as the starting material to give the crude product 2-(6-(trifluoromethyl)pyrazin-2-yl)-2,8-diazaspiro[4.5]decane hydrochloride (90 mg). MS m/z: 282 [M+H]⁺.

Step 3: 8-(1-(oxetan-3-yl)-1H-pyrazolo[3,4-b]pyrazin-6-yl)-1-(6-(trifluoromethyl)pyrazin-2-yl)-2,8-diazaspiro[4.5]decane: Followed the general procedure I using 6-chloro-1-(oxetan-3-yl)pyrazolo[3,4-b]pyrazine (60 mg, 285 μmol, 1.0 equiv.) and 2-[6-(trifluoromethyl)pyrazin-2-yl]-2,8-diazaspiro[4.5]decane hydrogen chloride (89 mg, 314 μmol, 1.1 equiv.) as the starting materials to give 8-(1-(oxetan-3-yl)-1H-pyrazolo[3,4-b]pyrazin-6-yl)-2-(6-(trifluoromethyl)pyrazin-2-yl)-2,8-diazaspiro[4.5]decane (37.7 mg, 28%) as a white solid. ¹H NMR (400 MHz, DMSO-d₆) δ 8.48 (s, 1H), 8.30 (s, 1H), 8.18 (d, J=7.0 Hz, 2H), 5.95-5.86 (m, 1H), 5.06 (t, J=6.4 Hz, 2H), 4.98 (dd, J=7.9, 6.3 Hz, 2H), 3.90-3.75 (m, 4H), 3.61 (t, J=7.0 Hz, 2H), 3.46 (s, 2H), 2.02-1.93 (m, 2H), 1.77-1.61 (m, 4H). MS m/z: 461.10 [M+H]⁺.

### Example 769: 8-(1-(oxetan-3-yl)-1H-pyrazolo[3,4-b]pyrazin-6-yl)-2-(2-(trifluoromethyl)pyridin-4-yl)-2,8-diazaspiro[4.5]decan-3-one

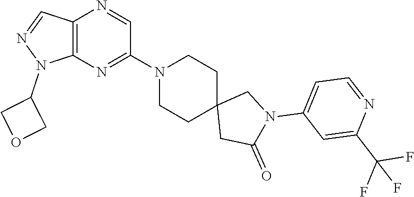 [SMILES: O=C1CC2(CCN(c3cnc4cnn(C5COC5)c4n3)CC2)CN1c1ccnc(C(F)(F)F)c1]

Step 1: tert-butyl 3-oxo-2-(2-(trifluoromethyl)pyridin-4-yl)-2,8-diazaspiro[4.5]decane-8-carboxylate: Followed the general procedure I using 4-chloro-2-(trifluoromethyl)pyridine (100 mg, 0.549 mmol, 1.0 equiv.) and tert-butyl 3-oxo-2,8-diazaspiro[4.5]decane-8-carboxylate (154 mg, 0.604 mmol, 1.1 equiv.) as the starting materials to give tert-butyl 3-oxo-2-(2-(trifluoromethyl)pyridin-4-yl)-2,8-diazaspiro[4.5]decane-8-carboxylate (70.0 mg, 31.9%) as a light yellow oil. MS m/z: 400 [M+H]⁺.

Step 2: 2-(2-(trifluoromethyl)pyridin-4-yl)-2,8-diazaspiro[4.5]decan-3-one hydrochloride: Followed the general procedure B using tert-butyl 3-oxo-2-(2-(trifluoromethyl)pyridin-4-yl)-2,8-diazaspiro[4.5]decane-8-carboxylate (70.0 mg, 0.175 mmol, 1.0 equiv.) as the starting materials to give the crude product tert-butyl 3-oxo-2-(2-(trifluoromethyl)pyridin-4-yl)-2,8-diazaspiro[4.5]decane-8-carboxylate (50.0 mg) as a light yellow solid. MS m/z: 300 [M+H]⁺

Step 3: 8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-3-one: Followed the general procedure I using tert-butyl 3-oxo-2-(2-(trifluoromethyl)pyridin-4-yl)-2,8-diazaspiro[4.5]decane-8-carboxylate (50.0 mg, 0.167 mmol, 1.0 equiv.) and 6-chloro-1-(oxetan-3-yl)-1H-pyrazolo[3,4-b]pyrazine (35.2 mg, 0.133 mmol, 1.0 equiv.) as the starting materials to give 8-(1-(oxetan-3-yl)-1H-pyrazolo[3,4-b]pyrazin-6-yl)-2-(2-(trifluoromethyl)pyridin-4-yl)-2,8-diazaspiro[4.5]decan-3-one (13.4 mg, 16.9%) as a light yellow solid. ¹H NMR (400 MHz, DMSO-d₆) δ 8.69 (d, J=5.6 Hz, 1H), 8.49 (s, 1H), 8.27 (d, J=2.1 Hz, 1H), 8.19 (s, 1H), 7.88 (dd, J=5.6, 2.2 Hz, 1H), 5.96-5.85 (m, 1H), 5.05 (t, J=6.4 Hz, 2H), 4.97 (dd, J=7.9, 6.4 Hz, 2H), 3.96-3.84 (m, 4H), 3.82-3.73 (m, 2H), 2.66 (s, 2H), 1.74 (s, 4H). MS m/z: 474.2 [M+H]⁺.

### Example 770: 1-(2,2-difluoroethyl)-6-(7-(5-(trifluoromethyl)pyrazin-2-yl)-2,7-diazaspiro[3.5]nonan-2-yl)-1H-pyrazolo[3,4-b]pyrazine

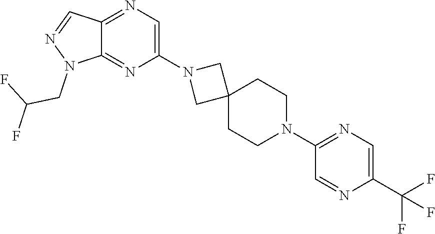 [SMILES: FC(F)Cn1ncc2ncc(N3CC4(CCN(c5cnc(C(F)(F)F)cn5)CC4)C3)nc21]

Step 1: tert-butyl 7-(5-(trifluoromethyl)pyrazin-2-yl)-2,7-diazaspiro[3.5]nonane-2-carboxylate: Followed the general procedure I using tert-butyl 2,7-diazaspiro[3.5]nonane-2-carboxylate (100 mg, 440 μmol, 1.0 equiv.) and 2-chloro-5-(trifluoromethyl)pyrazine (97 mg, 520 μmol, 1.2 equiv.) as the starting materials to give tert-butyl 7-(5-(trifluoromethyl)pyrazin-2-yl)-2,7-diazaspiro[3.5]nonane-2-carboxylate (60 mg, 37%) as a white solid. MS m/z: 373 [M+H]⁺.

Step 2: 7-(5-(trifluoromethyl)pyrazin-2-yl)-2,7-diazaspiro[3.5]nonane hydrochloride: Followed the general procedure B using tert-butyl 7-(5-(trifluoromethyl)pyrazin-2-yl)-2,7-diazaspiro[3.5]nonane-2-carboxylate (60 mg) as the starting material to give the crude product 7-(5-(trifluoromethyl)pyrazin-2-yl)-2,7-diazaspiro[3.5]nonane hydrochloride (60 mg). MS m/z: 273 [M+H]⁺.

Step 3: 1-(2,2-difluoroethyl)-6-(7-(5-(trifluoromethyl)pyrazin-2-yl)-2,7-diazaspiro[3.5]nonan-2-yl)-1H-pyrazolo[3,4-b]pyrazine: Followed the general procedure I using 7-(5-(trifluoromethyl)pyrazin-2-yl)-2,7-diazaspiro[3.5]nonane hydrochloride (60 mg, 195 μmol, 1.0 equiv.) and 6-chloro-1-(2,2-difluoroethyl)-1H-pyrazolo[3,4-b]pyrazine (71 mg, 215 μmol, 1.1 equiv.) as the starting materials to give 1-(2,2-difluoroethyl)-6-(7-(5-(trifluoromethyl)pyrazin-2-yl)-2,7-diazaspiro[3.5]nonan-2-yl)-1H-pyrazolo[3,4-b]pyrazine (7.4 mg, 8%) as a white solid. ¹H NMR (400 MHz, DMSO-d₆) δ 8.52-8.46 (m, 2H), 8.15 (s, 1H), 7.94 (s, 1H), 6.58-6.30 (m, 1H), 4.67 (td, J=15.0, 3.8 Hz, 2H), 4.00 (s, 4H), 3.78 (d, J=5.6 Hz, 4H), 1.88 (t, J=5.2 Hz, 4H). MS m/z: 455.1 [M+H]⁺.

### Example 771: 1-(2,2-difluoroethyl)-6-(7-(6-(trifluoromethyl)pyrazin-2-yl)-2,7-diazaspiro[3.5]nonan-2-yl)-1H-pyrazolo[3,4-b]pyrazine

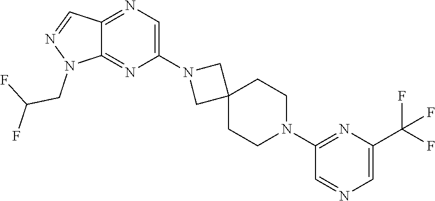 [SMILES: FC(F)Cn1ncc2ncc(N3CC4(CCN(c5cncc(C(F)(F)F)n5)CC4)C3)nc21]

Step 1: tert-butyl 7-(6-(trifluoromethyl)pyrazin-2-yl)-2,7-diazaspiro[3.5]nonane-2-carboxylate: Followed the general procedure I using tert-butyl 2,7-diazaspiro[3.5]nonane-2-carboxylate (100 mg, 450 μmol, 1.0 equiv.) and 2-bromo-6-(trifluoromethyl)pyrazine (102 mg, 540 μmol, 1.2 equiv.) as the starting materials to give tert-butyl 7-(6-(trifluoromethyl)pyrazin-2-yl)-2,7-diazaspiro[3.5]nonane-2-carboxylate (70 mg, 43%) as a white solid. MS m/z: 373 [M+H]⁺.

Step 2: 7-(6-(trifluoromethyl)pyrazin-2-yl)-2,7-diazaspiro[3.5]nonane hydrochloride: Followed the general procedure B using tert-butyl 7-(6-(trifluoromethyl)pyrazin-2-yl)-2,7-diazaspiro[3.5]nonane-2-carboxylate (70 mg, 188 μmol, 1.0 equiv. 1) as the starting material to give the crude product 7-(6-(trifluoromethyl)pyrazin-2-yl)-2,7-diazaspiro[3.5]nonane hydrochloride (70 mg). MS m/z: 273 [M+H]⁺.

Step 3: 1-(2,2-difluoroethyl)-6-(7-(6-(trifluoromethyl)pyrazin-2-yl)-2,7-diazaspiro[3.5]nonan-2-yl)-1H-pyrazolo[3,4-b]pyrazine: Followed the general procedure I using 7-(6-(trifluoromethyl)pyrazin-2-yl)-2,7-diazaspiro[3.5]nonane hydrogen chloride (70 mg, 171 μmol, 1.0 equiv.) and 6-chloro-1-(2,2-difluoroethyl)-1H-pyrazolo[3,4-b]pyrazine (83 mg, 342 μmol, 2.0 equiv.) as the starting materials to give 1-(2,2-difluoroethyl)-6-(7-(6-(trifluoromethyl)pyrazin-2-yl)-2,7-diazaspiro[3.5]nonan-2-yl)-1H-pyrazolo[3,4-b]pyrazine (70 mg, 43%) as a white solid. ¹H NMR (400 MHz, DMSO-d₆) δ 8.70 (s, 1H), 8.20 (s, 1H), 8.15 (s, 1H), 7.93 (s, 1H), 6.58-6.31 (m, 1H), 4.72-4.63 (m, 2H), 3.99 (s, 4H), 3.71 (t, J=5.4 Hz, 4H), 1.88 (t, J=5.4 Hz, 4H). MS m/z: 455.2 [M+H]⁺.

### Example 772: 2-(1-(2,2-difluoroethyl)-1H-pyrazolo[3,4-b]pyrazin-6-yl)-8-(5-(trifluoromethyl)pyrazin-2-yl)-2,8-diazaspiro[4.5]decan-1-one

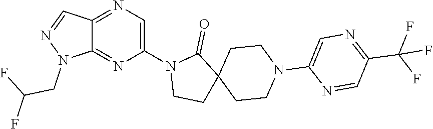 [SMILES: O=C1N(c2cnc3cnn(CC(F)F)c3n2)CCC12CCN(c1cnc(C(F)(F)F)cn1)CC2]

Step 1: tert-butyl 3-oxo-2-(6-(trifluoromethyl)pyridin-3-yl)-2,8-diazaspiro[4.5]decane-8-carboxylate: Followed the general procedure I using tert-butyl 1-oxo-2,8-diazaspiro[4.5]decane-8-carboxylate (100 mg, 0.392 mmol, 1.0 equiv.) and tert-butyl 1-oxo-2,8-diazaspiro[4.5]decane-8-carboxylate (94.5 mg, 0.432 mmol, 1.1 equiv.) as the starting materials, Xphos Pd G3 (33.2 mg, 0.039 mmol, 0.1 equiv.) as the catalyst to give tert-butyl 2-(1-(2,2-difluoroethyl)-1H-pyrazolo[3,4-b]pyrazin-6-yl)-1-oxo-2,8-diazaspiro[4.5]decane-8-carboxylate (118 mg, 68%) as a white solid. MS m/z: 437 [M+H]⁺.

Step 2: 2-(1-(2,2-difluoroethyl)-1H-pyrazolo[3,4-b]pyrazin-6-yl)-2,8-diazaspiro[4.5]decan-1-one hydrochloride: Followed the general procedure B using tert-butyl 2-(1-(2,2-difluoroethyl)-1H-pyrazolo[3,4-b]pyrazin-6-yl)-1-oxo-2,8-diazaspiro[4.5]decane-8-carboxylate (118 mg) as the starting material to give the crude product 2-(1-(2,2-difluoroethyl)-1H-pyrazolo[3,4-b]pyrazin-6-yl)-2,8-diazaspiro[4.5]decan-1-one hydrochloride (70 mg). MS m/z: 337 [M+H]⁺.

Step 3: 2-(1-(2,2-difluoroethyl)-1H-pyrazolo[3,4-b]pyrazin-6-yl)-8-(5-(trifluoromethyl)pyrazin-2-yl)-2,8-diazaspiro[4.5]decan-1-one: Followed the general procedure I using 2-(1-(2,2-difluoroethyl)-1H-pyrazolo[3,4-b]pyrazin-6-yl)-2,8-diazaspiro[4.5]decan-1-one hydrochloride (70 mg, 0.208 mmol, 1.00 equiv.) and 6-chloro-1-(2,2-difluoroethyl)-1H-pyrazolo[3,4-b]pyrazine (45.7 mg, 0.249 mmol, 1.20 equiv.) as the starting materials to give 2-(1-(2,2-difluoroethyl)-1H-pyrazolo[3,4-b]pyrazin-6-yl)-8-(5-(trifluoromethyl)pyrazin-2-yl)-2,8-diazaspiro[4.5]decan-1-one (47.9 mg, 47%) as a yellow green solid. ¹H NMR (400 MHz, DMSO-d₆) δ 9.67 (s, 1H), 8.50 (d, J=2.1 Hz, 3H), 6.72-6.37 (m, 1H), 4.95-4.82 (m, 2H), 4.38 (d, J=13.7 Hz, 2H), 4.12 (t, J=7.1 Hz, 2H), 3.41 (d, J=3.3 Hz, 2H), 2.28 (t, J=7.1 Hz, 2H), 1.90-1.71 (m, 4H). MS m/z: 483.05 [M+H]⁺.

### Example 773: 8-(1-(2,2-difluoroethyl)-1H-pyrazolo[3,4-b]pyrazin-6-yl)-2-(4-(trifluoromethyl)pyrimidin-2-yl)-2,8-diazaspiro[4.5]decan-1-one

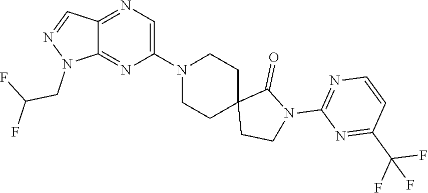 [SMILES: O=C1N(c2nccc(C(F)(F)F)n2)CCC12CCN(c1cnc3cnn(CC(F)F)c3n1)CC2]

Step 1: tert-butyl 1-oxo-2-(4-(trifluoromethyl)pyrimidin-2-yl)-2,8-diazaspiro[4.5]decane-8-carboxylate: Followed the general procedure I using tert-butyl 1-oxo-2,8-diazaspiro[4.5]decane-8-carboxylate (70 mg, 0.275 mmol, 1.0 equiv.) and 2-chloro-4-(trifluoromethyl)pyrimidine (50 mg, 0.275 mmol, 1.0 equiv.) as the starting materials to give tert-butyl 1-oxo-2-(4-(trifluoromethyl)pyrimidin-2-yl)-2,8-diazaspiro[4.5]decane-8-carboxylate (70 mg, 63%) as a white solid. MS m/z: 401 [M+H]⁺.

Step 2: 2-(4-(trifluoromethyl)pyrimidin-2-yl)-2,8-diazaspiro[4.5]decan-1-one hydrochloride: Followed the general procedure B using tert-butyl 1-oxo-2-(4-(trifluoromethyl)pyrimidin-2-yl)-2,8-diazaspiro[4.5]decane-8-carboxylate (70 mg) as the starting material to give the crude product 2-(4-(trifluoromethyl)pyrimidin-2-yl)-2,8-diazaspiro[4.5]decan-1-one hydrochloride (50 mg). MS m/z: 301 [M+H]⁺.

Step 3: 8-(1-(2,2-difluoroethyl)-1H-pyrazolo[3,4-b]pyrazin-6-yl)-2-(4-(trifluoromethyl)pyrimidin-2-yl)-2,8-diazaspiro[4.5]decan-1-one: Followed the general procedure I using 2-(4-(trifluoromethyl)pyrimidin-2-yl)-2,8-diazaspiro[4.5]decan-1-one hydrochloride (50 mg, 0.167 mmol, 1 equiv.) and 6-chloro-1-(2,2-difluoroethyl)-1H-pyrazolo[3,4-b]pyrazine (37 mg, 0.203 mmol, 1 equiv.) as the starting materials to give 8-(1-(2,2-difluoroethyl)-1H-pyrazolo[3,4-b]pyrazin-6-yl)-2-(4-(trifluoromethyl)pyrimidin-2-yl)-2,8-diazaspiro[4.5]decan-1-one (23.4 mg, 29%) as a white solid. ¹H NMR (400 MHz, DMSO-d₆) δ 9.10 (d, J=4.9 Hz, 1H), 8.52 (s, 1H), 8.15 (s, 1H), 7.73 (d, J=5.0 Hz, 1H), 6.45 (tt, J=54.9, 3.8 Hz, 1H), 4.77-4.64 (m, 2H), 4.43 (dd, J=13.9, 4.1 Hz, 2H), 4.03 (t, J=7.0 Hz, 2H), 3.41-3.34 (m, 2H), 2.20 (t, J=7.0 Hz, 2H), 1.89-1.77 (m, 2H), 1.77-1.69 (m, 2H). MS m/z: 483.1 [M+H]⁺.

### Example 774: 8-(1-(2,2-difluoroethyl)-1H-pyrazolo[3,4-b]pyrazin-6-yl)-2-(6-(trifluoromethyl)pyrazin-2-yl)-2,8-diazaspiro[4.5]decan-1-one

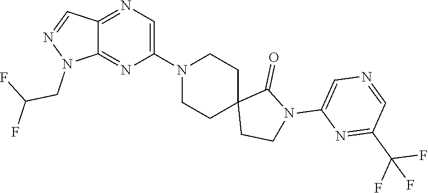 [SMILES: O=C1N(c2cncc(C(F)(F)F)n2)CCC12CCN(c1cnc3cnn(CC(F)F)c3n1)CC2]

Step 1: tert-butyl 1-oxo-2-(6-(trifluoromethyl)pyrazin-2-yl)-2,8-diazaspiro[4.5]decane-8-carboxylate: Followed the general procedure I using tert-butyl 1-oxo-2,8-diazaspiro[4.5]decane-8-carboxylate (100 mg, 390 μmol, 1.0 equiv.) and 2-chloro-6-(trifluoromethyl)pyrazine (86 mg, 470 μmol, 1.2 equiv.) as the starting materials to give tert-butyl 1-oxo-2-(6-(trifluoromethyl)pyrazin-2-yl)-2,8-diazaspiro[4.5]decane-8-carboxylate (80 mg, 52%) as a white solid. MS m/z: 401 [M+H]⁺.

Step 2: 2-(6-(trifluoromethyl)pyrazin-2-yl)-2,8-diazaspiro[4.5]decan-1-one hydrochloride: Followed the general procedure B using tert-butyl 1-oxo-2-(6-(trifluoromethyl)pyrazin-2-yl)-2,8-diazaspiro[4.5]decane-8-carboxylate (80 mg) as the starting material to give the crude product 2-(6-(trifluoromethyl)pyrazin-2-yl)-2,8-diazaspiro[4.5]decan-1-one hydrochloride (80 mg). MS m/z: 301 [M+H]⁺.

Step 3: 8-(1-(2,2-difluoroethyl)-1H-pyrazolo[3,4-b]pyrazin-6-yl)-2-(6-(trifluoromethyl)pyrazin-2-yl)-2,8-diazaspiro[4.5]decan-1-one: Followed the general procedure I using 6-chloro-1-(2,2-difluoroethyl)-1H-pyrazolo[3,4-b]pyrazine (60 mg, 274 μmol, 1.0 equiv.) and 2-(6-(trifluoromethyl)pyrazin-2-yl)-2,8-diazaspiro[4.5]decan-1-one hydrochloride (90 mg, 301 μmol, 1.1 equiv.) as the starting materials to give 8-(1-(2,2-difluoroethyl)-1H-pyrazolo[3,4-b]pyrazin-6-yl)-2-(6-(trifluoromethyl)pyrazin-2-yl)-2,8-diazaspiro[4.5]decan-1-one (36.7 mg, 27%) as a white solid. ¹H NMR (400 MHz, DMSO-d₆) δ 9.77 (s, 1H), 8.82 (s, 1H), 8.44 (s, 1H), 8.10 (s, 1H), 6.38 (tt, J=54.8, 3.6 Hz, 1H), 4.68 (td, J=15.2, 3.6 Hz, 2H), 4.45-4.33 (m, 2H), 3.97 (d, J=7.0 Hz, 2H), 3.32 (ddd, J=14.0, 11.0, 3.0 Hz, 2H), 2.24 (t, J=7.2 Hz, 2H), 1.82 (ddd, J=15.0, 10.8, 3.8 Hz, 2H), 1.72 (d, J=13.6 Hz, 2H). MS m/z: 483.05 [M+H]⁺.

### Example 775: 8-[1-(2,2-difluoroethyl)pyrazolo[3,4-b]pyrazin-6-yl]-2-[3-methyl-5-(trifluoromethyl)pyridin-2-yl]-2,8-diazaspiro[4.5]decan-1-one

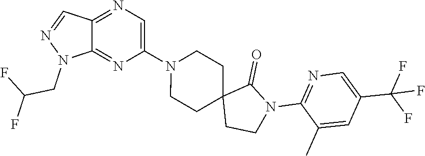 [SMILES: Cc1cc(C(F)(F)F)cnc1N1CCC2(CCN(c3cnc4cnn(CC(F)F)c4n3)CC2)C1=O]

Step 1: tert-butyl 2-[3-bromo-5-(trifluoromethyl)pyridin-2-yl]-1-oxo-2,8-diazaspiro[4.5]decane-8-carboxylate: Followed the general procedure I using tert-butyl 1-oxo-2,8-diazaspiro[4.5]decane-8-carboxylate (200 mg, 0.786 mmol, 1.0 equiv.) and 3-bromo-2-chloro-5-(trifluoromethyl)pyridine (225 mg, 0.865 mmol, 1.1 equiv.) as the starting materials to give tert-butyl 2-[3-bromo-5-(trifluoromethyl)pyridin-2-yl]-1-oxo-2,8-diazaspiro[4.5]decane-8-carboxylate (100 mg, 26%) as a white solid. MS m/z: 478 [M+H]⁺.

Step 2: tert-butyl 2-[3-methyl-5-(trifluoromethyl)pyridin-2-yl]-1-oxo-2,8-diazaspiro[4.5]decane-8-carboxylate: Followed the general procedure L using tert-butyl 2-[3-bromo-5-(trifluoromethyl)pyridin-2-yl]-1-oxo-2,8-diazaspiro[4.5]decane-8-carboxylate (100 mg, 0.209 mmol, 1.0 equiv.) and methylboronic acid (62.5 mg, 1.045 mmol, 5.0 equiv.) as the starting materials to give tert-butyl 2-[3-methyl-5-(trifluoromethyl)pyridin-2-yl]-1-oxo-2,8-diazaspiro[4.5]decane-8-carboxylate (80 mg, 92%) as a light yellow solid. MS m/z: 414 [M+H]⁺.

Step 3: 2-[3-methyl-5-(trifluoromethyl)pyridin-2-yl]-2,8-diazaspiro[4.5]decan-1-one hydrochloride: Followed the general procedure B using tert-butyl 2-[3-methyl-5-(trifluoromethyl)pyridin-2-yl]-1-oxo-2,8-diazaspiro[4.5]decane-8-carboxylate (80 mg) as the starting material to give the crude product 2-[3-methyl-5-(trifluoromethyl)pyridin-2-yl]-2,8-diazaspiro[4.5]decan-1-one hydrochloride (80 mg) as a white solid. MS m/z: 314 [M+H]⁺.

Step 4: 8-[1-(2,2-difluoroethyl)pyrazolo[3,4-b]pyrazin-6-yl]-2-[3-methyl-5-(trifluoromethyl)pyridin-2-yl]-2,8-diazaspiro[4.5]decan-1-one: Followed the general procedure I using 2-[3-methyl-5-(trifluoromethyl)pyridin-2-yl]-2,8-diazaspiro[4.5]decan-1-one hydrochloride (80 mg, 0.229 mmol, 1.0 equiv.) and 6-chloro-1-(2,2-difluoroethyl)pyrazolo[3,4-b]pyrazine (50 mg, 0.229 mmol, 1 equiv.) as the starting materials to give 8-[1-(2,2-difluoroethyl)pyrazolo[3,4-b]pyrazin-6-yl]-2-[3-methyl-5-(trifluoromethyl)pyridin-2-yl]-2,8-diazaspiro[4.5]decan-1-one (55 mg, 48%) as a white solid. ¹H NMR (300 MHz, DMSO-d₆) δ 8.73 (s, 1H), 8.52 (s, 1H), 8.21 (s, 1H), 8.15 (s, 1H), 6.43 (tt, 1H), 4.71 (td, 2H), 4.47-4.42 (m, 2H), 4.00-3.95 (m, 2H), 3.48-3.31 (m, 2H), 2.32-2.25 (m, 5H), 1.91-1.78 (m, 2H), 1.75-1.70 (m, 2H). MS m/z: 496.10 [M+H]⁺.

### Example 776: 2-(3-chloro-5-(trifluoromethyl)pyridin-2-yl)-8-(1-(2,2-difluoroethyl)-1H-pyrazolo[3,4-b]pyrazin-6-yl)-2,8-diazaspiro[4.5]decan-1-one

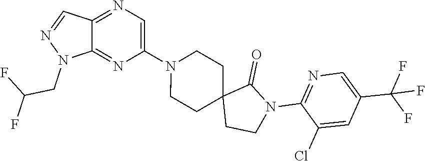 [SMILES: O=C1N(c2ncc(C(F)(F)F)cc2Cl)CCC12CCN(c1cnc3cnn(CC(F)F)c3n1)CC2]

Step 1: tert-butyl 2-(3-chloro-5-(trifluoromethyl)pyridin-2-yl)-1-oxo-2,8-diazaspiro[4.5]decane-8-carboxylate: Followed the general procedure E using tert-butyl 1-oxo-2,8-diazaspiro[4.5]decane-8-carboxylate (100 mg, 0.393 mmol, 1 equiv) and 2,3-dichloro-5-(trifluoromethyl)pyridine (101 mg, 0.472 mmol, 1.2 equiv) as the starting materials to give tert-butyl 2-(3-chloro-5-(trifluoromethyl)pyridin-2-yl)-1-oxo-2,8-diazaspiro[4.5]decane-8-carboxylate (75 mg, 44%) as a white solid. MS m/z: 434 [M+H]⁺.

Step 2: 2-(3-chloro-5-(trifluoromethyl)pyridin-2-yl)-2,8-diazaspiro[4.5]decan-1-one hydrochloride: Followed the general procedure B using tert-butyl 2-(3-chloro-5-(trifluoromethyl)pyridin-2-yl)-1-oxo-2,8-diazaspiro[4.5]decane-8-carboxylate (75 mg, 0.173 mmol, 1 equiv) as the starting material to give the crude product 2-(3-chloro-5-(trifluoromethyl)pyridin-2-yl)-2,8-diazaspiro[4.5]decan-1-one hydrochloride (50 mg) MS m/z: 334 [M+H]⁺.

Step 3: 2-(3-chloro-5-(trifluoromethyl)pyridin-2-yl)-8-(1-(2,2-difluoroethyl)-1H-pyrazolo[3,4-b]pyrazin-6-yl)-2,8-diazaspiro[4.5]decan-1-one: Followed the general procedure I using 2-(3-chloro-5-(trifluoromethyl)pyridin-2-yl)-2,8-diazaspiro[4.5]decan-1-one hydrochloride (50 mg, 0.150 mmol, 1 equiv) and 6-chloro-1-(2,2-difluoroethyl)-1H-pyrazolo[3,4-b]pyrazine (40 mg, 0.180 mmol, 1.2 equiv) as the starting materials to give 2-(3-chloro-5-(trifluoromethyl)pyridin-2-yl)-8-(1-(2,2-difluoroethyl)-1H-pyrazolo[3,4-b]pyrazin-6-yl)-2,8-diazaspiro[4.5]decan-1-one (30 mg, 39%) as a white solid. ¹H NMR (400 MHz, DMSO-d₆) δ 8.91 (s, 1H), 8.63-8.58 (m, 1H), 8.51 (s, 1H), 8.17-8.12 (m, 1H), 6.45 (tt, J=54.9, 3.9 Hz, 1H), 4.71 (td, J=15.0, 3.8 Hz, 2H), 4.41 (dt, J=13.9, 4.5 Hz, 2H), 3.96 (t, J=6.9 Hz, 2H), 3.45-3.37 (m, 2H), 2.31 (t, J=6.9 Hz, 2H), 1.89-1.72 (m, 2H), 1.76-1.67 (m, 2H). MS m/z: 516.0 [M+H]⁺.

### Example 777: 8-(1-(2,2-difluoroethyl)-1H-pyrazolo[3,4-b]pyrazin-6-yl)-2-(3-fluoro-5-(trifluoromethyl)pyridin-2-yl)-2,8-diazaspiro[4.5]decan-1-one

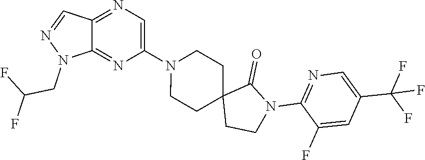 [SMILES: O=C1N(c2ncc(C(F)(F)F)cc2F)CCC12CCN(c1cnc3cnn(CC(F)F)c3n1)CC2]

Step 1: tert-butyl 2-(3-fluoro-5-(trifluoromethyl)pyridin-2-yl)-1-oxo-2,8-diazaspiro[4.5]decane-8-carboxylate: Followed the general procedure I using tert-butyl 1-oxo-2,8-diazaspiro[4.5]decane-8-carboxylate (100 mg, 0.393 mmol, 1.0 equiv.) and 2,3-difluoro-5-(trifluoromethyl)pyridine (72 mg, 0.393 mmol, 1.0 equiv.) as the starting materials to give tert-butyl 2-(3-fluoro-5-(trifluoromethyl)pyridin-2-yl)-1-oxo-2,8-diazaspiro[4.5]decane-8-carboxylate (70 mg, 42%) as a white solid. MS m/z: 418 [M+H]⁺.

Step 2: 2-(3-fluoro-5-(trifluoromethyl)pyridin-2-yl)-2,8-diazaspiro[4.5]decan-1-one hydrochloride: Followed the general procedure B using tert-butyl 2-(3-fluoro-5-(trifluoromethyl)pyridin-2-yl)-1-oxo-2,8-diazaspiro[4.5]decane-8-carboxylate (70 mg) as the starting material to give the crude product 2-(3-fluoro-5-(trifluoromethyl)pyridin-2-yl)-2,8-diazaspiro[4.5]decan-1-one hydrochloride (50 mg). MS m/z: 318 [M+H]⁺.

Step 3: 8-(1-(2,2-difluoroethyl)-1H-pyrazolo[3,4-b]pyrazin-6-yl)-2-(3-fluoro-5-(trifluoromethyl)pyridin-2-yl)-2,8-diazaspiro[4.5]decan-1-one: Followed the general procedure I using 2-(3-fluoro-5-(trifluoromethyl)pyridin-2-yl)-2,8-diazaspiro[4.5]decan-1-one hydrochloride (50 mg, 0.157 mmol, 1.0 equiv.) and 6-chloro-1-(2,2-difluoroethyl)-1H-pyrazolo[3,4-b]pyrazine (35 mg, 0.157 mmol, 1.0 equiv.) as the starting materials to give 8-(1-(2,2-difluoroethyl)-1H-pyrazolo[3,4-b]pyrazin-6-yl)-2-(4-(trifluoromethyl)pyrimidin-2-yl)-2,8-diazaspiro[4.5]decan-1-one (31.4 mg, 39%) as a white solid. ¹H NMR (400 MHz, DMSO-d₆) δ 8.76 (d, J=2.0 Hz, 1H), 8.52 (s, 1H), 8.43 (dd, J=10.0, 2.0 Hz, 1H), 8.15 (s, 1H), 6.45 (tt, J=54.9, 3.8 Hz, 1H), 4.77-4.64 (m, 2H), 4.47-4.37 (m, 2H), 4.00 (t, J=6.9 Hz, 2H), 3.44-3.37 (m, 2H), 2.30 (t, J=6.9 Hz, 2H), 1.88-1.77 (m, 2H), 1.77-1.68 (m, 2H). MS m/z: 500.10 [M+H]⁺.

### Example 778: 8-(1-(2,2-difluoroethyl)-1H-pyrazolo[3,4-b]pyrazin-6-yl)-2-(4-methyl-6-(trifluoromethyl)pyridin-3-yl)-2,8-diazaspiro[4.5]decan-3-one

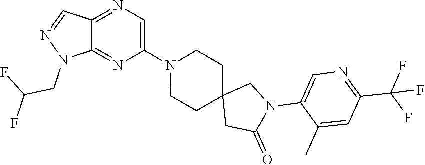 [SMILES: Cc1cc(C(F)(F)F)ncc1N1CC2(CCN(c3cnc4cnn(CC(F)F)c4n3)CC2)CC1=O]

Step 1: tert-butyl 2-(4-methyl-6-(trifluoromethyl)pyridin-3-yl)-3-oxo-2,8-diazaspiro[4.5]decane-8-carboxylate: Followed the general procedure Y using tert-butyl 3-oxo-2,8-diazaspiro[4.5]decane-8-carboxylate (100 mg, 393 μmol, 1.0 equiv.) and 5-bromo-4-methyl-2-(trifluoromethyl)pyridine (94 mg, 393 μmol, 1.0 equiv.) as the starting materials to give tert-butyl 2-(4-methyl-6-(trifluoromethyl)pyridin-3-yl)-3-oxo-2,8-diazaspiro[4.5]decane-8-carboxylate (40 mg, 25%) as a white solid. MS m/z: 414 [M+H]⁺.

Step 2: 2-(4-methyl-6-(trifluoromethyl)pyridin-3-yl)-2,8-diazaspiro[4.5]decan-3-one hydrochloride: Followed the general procedure B using tert-butyl 2-(4-methyl-6-(trifluoromethyl)pyridin-3-yl)-3-oxo-2,8-diazaspiro[4.5]decane-8-carboxylate (40 mg) as the starting material to give the crude product 2-(4-methyl-6-(trifluoromethyl)pyridin-3-yl)-2,8-diazaspiro[4.5]decan-3-one hydrochloride (40 mg). MS m/z: 314 [M+H]⁺.

Step 3: 8-(1-(2,2-difluoroethyl)-1H-pyrazolo[3,4-b]pyrazin-6-yl)-2-(4-methyl-6-(trifluoromethyl)pyridin-3-yl)-2,8-diazaspiro[4.5]decan-3-one: Followed the general procedure I using 2-(4-methyl-6-(trifluoromethyl)pyridin-3-yl)-2,8-diazaspiro[4.5]decan-3-one hydrochloride (40 mg, 0.13 mmol, 1.0 equiv.) and 6-chloro-1-(2,2-difluoroethyl)-1H-pyrazolo[3,4-b]pyrazine (27.8 mg, 0.11 mmol, 1.0 equiv.) as the starting materials to give 8-(1-(2,2-difluoroethyl)-1H-pyrazolo[3,4-b]pyrazin-6-yl)-2-(4-methyl-6-(trifluoromethyl)pyridin-3-yl)-2,8-diazaspiro[4.5]decan-3-one (10.2 mg, 16%) as a white solid. ¹H NMR (400 MHz, DMSO-d₆) δ 8.49 (s, 1H), 8.40 (s, 1H), 8.13 (s, 1H), 7.52 (s, 1H), 6.44 (tt, J=54.9, 3.8 Hz, 1H), 4.70 (td, J=14.9, 3.9 Hz, 2H), 4.10-4.02 (m, 2H), 3.87 (s, 2H), 3.62-3.52 (s, 2H), 2.76-2.66 (m, 2H), 2.46 (s, 3H), 1.81-1.71 (m, 4H). MS m/z: 496.2 [M+H]⁺.

### Example 779: 8-(1-(2,2-difluoroethyl)-1H-pyrazolo[3,4-b]pyrazin-6-yl)-2-(2-methoxy-6-(trifluoromethyl)pyridin-3-yl)-2,8-diazaspiro[4.5]decan-3-one

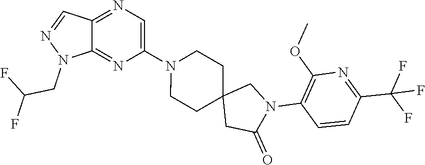 [SMILES: COc1nc(C(F)(F)F)ccc1N1CC2(CCN(c3cnc4cnn(CC(F)F)c4n3)CC2)CC1=O]

Step 1: 3-bromo-6-(trifluoromethyl)pyridin-2-ol: Followed the general procedure V using 6-(trifluoromethyl)pyridin-2-ol (2500 mg, 15.2 mmol, 1.0 equiv.) as the starting material, NBS (2985 mg, 16.8 mmol, 1.1 equiv.) as the reagent to give 3-bromo-6-(trifluoromethyl)pyridin-2-ol (550 mg, 14.9%) as a white solid. MS m/z: 242 [M+H]⁺.

Step 2: 3-bromo-2-methoxy-6-(trifluoromethyl)pyridine: Followed the general procedure K using 3-bromo-6-(trifluoromethyl)pyridin-2-ol (550 mg, 2.27 mmol, 1.0 equiv.) and Mel (355 mg, 2.50 mmol, 1.0 equiv.) as the starting material to give 3-bromo-2-methoxy-6-(trifluoromethyl)pyridine (210 mg, 36.0%) as a white solid. MS m/z: 257 [M+H]⁺

Step 3: tert-butyl 2-(2-methoxy-6-(trifluoromethyl)pyridin-3-yl)-3-oxo-2,8-diazaspiro[4.5]decane-8-carboxylate: Followed the general procedure Y using 3-bromo-2-methoxy-6-(trifluoromethyl)pyridine (210 mg, 0.817 mmol, 1.0 equiv.) and tert-butyl 3-oxo-2,8-diazaspiro[4.5]decane-8-carboxylate (299 mg, 0.899 mmol, 1.2 equiv.) as the starting materials to give tert-butyl 2-(2-methoxy-6-(trifluoromethyl)pyridin-3-yl)-3-oxo-2,8-diazaspiro[4.5]decane-8-carboxylate (50.0 mg, 14.2%) as a light yellow oil. MS m/z: 430 [M+H]⁺.

Step 4: 2-(2-methoxy-6-(trifluoromethyl)pyridin-3-yl)-2,8-diazaspiro[4.5]decan-3-one hydrochloride: Followed the general procedure B using tert-butyl 2-(2-methoxy-6-(trifluoromethyl)pyridin-3-yl)-3-oxo-2,8-diazaspiro[4.5]decane-8-carboxylate (50.0 mg, 0.116 mmol, 1.0 equiv.) as the starting materials to give the crude product 2-(2-methoxy-6-(trifluoromethyl)pyridin-3-yl)-2,8-diazaspiro[4.5]decan-3-one hydrochloride (40.0 mg) as a light yellow solid. MS m/z: 330 [M+H]⁺.

Step 5: 8-(1-(2,2-difluoroethyl)-1H-pyrazolo[3,4-b]pyrazin-6-yl)-2-(2-methoxy-6-(trifluoromethyl)pyridin-3-yl)-2,8-diazaspiro[4.5]decan-3-one: Followed the general procedure I using 2-(2-methoxy-6-(trifluoromethyl)pyridin-3-yl)-2,8-diazaspiro[4.5]decan-3-one hydrochloride (40.0 mg, 0.121 mmol, 1.0 equiv.) and 6-chloro-1-(2,2-difluoroethyl)-1H-pyrazolo[3,4-b]pyrazine (29.2 mg, 0.133 mmol, 1.1 equiv.) as the starting materials to give 8-(1-(2,2-difluoroethyl)-1H-pyrazolo[3,4-b]pyrazin-6-yl)-2-(2-methoxy-6-(trifluoromethyl)pyridin-3-yl)-2,8-diazaspiro[4.5]decan-3-one (14.4 mg, 21.4%) as a white solid. ¹H NMR (400 MHz, DMSO-d₆) δ 8.50 (s, 1H), 8.14 (s, 1H), 8.04 (d, J=1.7 Hz, 1H), 7.19 (d, J=1.7 Hz, 1H), 6.44 (tt, J=54.9, 3.8 Hz, 1H), 4.70 (td, J=15.1, 3.8 Hz, 2H), 3.90 (s, 3H), 3.89-3.73 (m, 6H), 2.65 (s, 2H), 1.73 (d, J=5.9 Hz, 4H). MS m/z: 553.05 [M+H]⁺.

### Example 780: 8-(1-(2,2-difluoroethyl)-1H-pyrazolo[3,4-b]pyrazin-6-yl)-2-(2-(difluoromethoxy)-6-(trifluoromethyl)pyridin-3-yl)-2,8-diazaspiro[4.5]decan-3-one

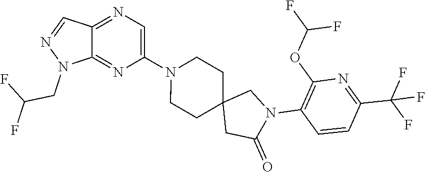 [SMILES: O=C1CC2(CCN(c3cnc4cnn(CC(F)F)c4n3)CC2)CN1c1ccc(C(F)(F)F)nc1OC(F)F]

Step 1: 3-bromo-2-(difluoromethoxy)-6-(trifluoromethyl)pyridine: A mixture of 3-bromo-6-(trifluoromethyl)pyridin-2-ol (600 mg, 2.5 mmol, 1.0 equiv.), methyl 2-chloro-2,2-difluoroacetate (540 mg, 3.75 mmol, 1.5 equiv.) and K₂CO₃ (1035 mg, 7.5 mmol, 3.0 equiv.) in DMF (10 mL) was heated at 90° C. 2 h. The reaction was monitored by LCMS. The mixture was diluted with water (50 mL), and extracted with EtOAc (50 mL×2). The organic layer was combined, washed with brine, dried, evaporated, and purified with a silico gel column, eluted with gradient of Hexane/EtOAc. The fractions were collected, and concentrated to give 3-bromo-2-(difluoromethoxy)-6-(trifluoromethyl)pyridine (380 mg, 55%) as a yellow oil. MS m/z: 292 [M+H]⁺.

Step 2: tert-butyl 2-(2-(difluoromethoxy)-6-(trifluoromethyl)pyridin-3-yl)-3-oxo-2,8-diazaspiro[4.5]decane-8-carboxylate: Followed the general procedure Y using 3-bromo-2-(difluoromethoxy)-6-(trifluoromethyl)pyridine (380 mg, 1.37 mmol, 1.0 equiv.) and tert-butyl 3-oxo-2,8-diazaspiro[4.5]decane-8-carboxylate (524 mg, 2.06 mmol, 1.5 equiv.) as the starting materials, Ephos Pd G4 (120 mg, 0.13 mmol, 0.1 equiv.) as the catalyst to give tert-butyl 2-(2-(difluoromethoxy)-6-(trifluoromethyl)pyridin-3-yl)-3-oxo-2,8-diazaspiro[4.5]decane-8-carboxylate (300 mg, 50%) as a yellow solid. MS m/z: 466 [M+H]⁺.

Step 3: 2-(2-(difluoromethoxy)-6-(trifluoromethyl)pyridin-3-yl)-2,8-diazaspiro[4.5]decan-3-one hydrochloride: Followed the general procedure B using tert-butyl 2-(2-(difluoromethoxy)-6-(trifluoromethyl)pyridin-3-yl)-3-oxo-2,8-diazaspiro[4.5]decane-8-carboxylate (300 mg) as the starting material to give the crude product 2-(2-(difluoromethoxy)-6-(trifluoromethyl)pyridin-3-yl)-2,8-diazaspiro[4.5]decan-3-one hydrochloride (300 mg). MS m/z: 366 [M+H]⁺.

Step 4: 8-(1-(2,2-difluoroethyl)-1H-pyrazolo[3,4-b]pyrazin-6-yl)-2-(2-(difluoromethoxy)-6-(trifluoromethyl)pyridin-3-yl)-2,8-diazaspiro[4.5]decan-3-one: Followed the general procedure I using 2-(2-(difluoromethoxy)-6-(trifluoromethyl)pyridin-3-yl)-2,8-diazaspiro[4.5]decan-3-one hydrochloride (120 mg, 0.30 mmol, 1.0 equiv.) and 6-chloro-1-(2,2-difluoroethyl)-1H-pyrazolo[3,4-b]pyrazine (98 mg, 0.50 mmol, 1.5 equiv.) as the starting materials to give 8-(1-(2,2-difluoroethyl)-1H-pyrazolo[3,4-b]pyrazin-6-yl)-2-(2-(difluoromethoxy)-6-(trifluoromethyl)pyridin-3-yl)-2,8-diazaspiro[4.5]decan-3-one (62.3 mg, 38%) as a white solid. ¹H NMR (400 MHz, DMSO-d₆) δ 8.51 (s, 1H), 8.24 (d, J=8.0 Hz, 1H), 8.13 (s, 1H), 7.91-7.51 (m, 2H), 6.44 (tt, J=54.9, 3.9 Hz, 1H), 4.70 (td, J=15.0, 3.9 Hz, 2H), 3.96-3.85 (m, 2H), 3.81-3.71 (m, 4H), 2.55 (s, 2H), 1.77 (t, J=5.7 Hz, 4H). MS m/z: 548.2 [M+H]⁺.

### Example 781: 7-[1-(2,2-difluoroethyl)-1H-pyrazolo[3,4-b]pyrazin-6-yl]-2-[4-(trifluoromethyl)pyridin-2-yl]-2,7-diazaspiro[3.5]nonane

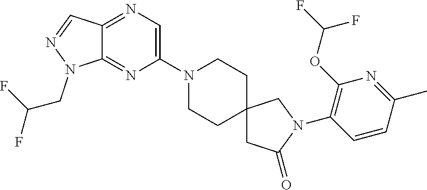 [SMILES: Cc1ccc(N2CC3(CCN(c4cnc5cnn(CC(F)F)c5n4)CC3)CC2=O)c(OC(F)F)n1]

Step 1: 3-bromo-2-(difluoromethoxy)-6-methylpyridine: A solution of 3-bromo-6-methylpyridin-2(1H)-one (100 mg, 0.532 mmol, 1.0 equiv.), K₂CO₃ (147 mg, 1.06 mmol, 2.0 equiv.) and methyl 2-chloro-2,2-difluoroacetate (115 mg, 0.798 mmol, 1.5 equiv.) in DMF (2 mL) was stirred for 4 h at 80° C. The resulting mixture was extracted with EtOAc (15 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/PE (1:1) to afford 3-bromo-2-(difluoromethoxy)-6-methylpyridine (80 mg, 63%) as a colorless oil. MS m/z: 238 [M+H]⁺.

Step 2: tert-butyl 2-(2-(difluoromethoxy)-6-methylpyridin-3-yl)-3-oxo-2,8-diazaspiro[4.5]decane-8-carboxylate: Followed the general procedure Y using 3-bromo-2-(difluoromethoxy)-6-methylpyridine (80 mg, 0.336 mmol, 1.0 equiv.) and tert-butyl 3-oxo-2,8-diazaspiro[4.5]decane-8-carboxylate (94.0 mg, 0.37 mmol, 1.1 equiv.) as the starting materials, Pd-PEPPSI-IPentCl 2-methylpyridine (o-picoline) (28.3 mg, 0.034 mmol, 0.1 equiv) as the catalyst to give tert-butyl 2-(2-(difluoromethoxy)-6-methylpyridin-3-yl)-3-oxo-2,8-diazaspiro[4.5]decane-8-carboxylate (90 mg, 65%) as a colorless oil. MS m/z: 412 [M+H]⁺.

Step 3: 2-(2-(difluoromethoxy)-6-methylpyridin-3-yl)-2,8-diazaspiro[4.5]decan-3-one hydrochloride: Followed the general procedure B using tert-butyl 2-(2-(difluoromethoxy)-6-methylpyridin-3-yl)-3-oxo-2,8-diazaspiro[4.5]decane-8-carboxylate (90 mg) as the starting material to give the crude product 2-(2-(difluoromethoxy)-6-methylpyridin-3-yl)-2,8-diazaspiro[4.5]decan-3-one hydrochloride (100 mg). MS m/z: 312 [M+H]⁺.

Step 4: 8-(1-(2,2-difluoroethyl)-1H-pyrazolo[3,4-b]pyrazin-6-yl)-2-(2-(difluoromethoxy)-6-methylpyridin-3-yl)-2,8-diazaspiro[4.5]decan-3-one: Followed the general procedure I using 8-(1-(2,2-difluoroethyl)-1H-pyrazolo[3,4-b]pyrazin-6-yl)-2-(2-(difluoromethoxy)-6-methylpyridin-3-yl)-2,8-diazaspiro[4.5]decan-3-one (50 mg, 0.16 mmol, 1.0 equiv.) and 6-chloro-1-(2,2-difluoroethyl)-1H-pyrazolo[3,4-b]pyrazine (38.4 mg, 0.175 mmol, 1.1 equiv.) as the starting materials to give 8-(1-(2,2-difluoroethyl)-1H-pyrazolo[3,4-b]pyrazin-6-yl)-2-(2-(difluoromethoxy)-6-methylpyridin-3-yl)-2,8-diazaspiro[4.5]decan-3-one (24 mg, 32%) as a white solid. ¹H NMR (400 MHz, DMSO-d₆) δ 8.50 (s, 1H), 8.13 (s, 1H), 7.87-7.47 (m, 2H), 7.20 (d, J=7.9 Hz, 1H), 6.44 (tt, J=54.9, 3.8 Hz, 1H), 4.70 (td, J=15.0, 3.8 Hz, 2H), 3.93-3.83 (m, 2H), 3.81-3.71 (m, 2H), 3.60 (s, 2H), 2.48 (s, 2H), 2.44 (s, 3H), 1.77 (t, J=5.6 Hz, 4H). m/z: 494.2 [M+H]⁺.

### Example 782: 8-(1-(2,2-difluoroethyl)-1H-pyrazolo[3,4-b]pyrazin-6-yl)-2-(6-methyl-2-(trifluoromethoxy)pyridin-3-yl)-2,8-diazaspiro[4.5]decan-3-one

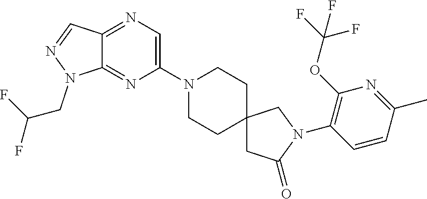 [SMILES: Cc1ccc(N2CC3(CCN(c4cnc5cnn(CC(F)F)c5n4)CC3)CC2=O)c(OC(F)(F)F)n1]

Step 1: 3-bromo-6-methyl-2-(trifluoromethoxy)pyridine: A solution of 3-bromo-6-methylpyridin-2(1H)-one (400 mg, 2.13 mmol, 1.0 equiv.) and 1-(trifluoromethyl)-1λ³-benzo[d][1,2]iodaoxol-3(1H)-one (1.01 g, 3.19 mmol, 1.5 equiv.) in CH₃NO₂ (10 mL) was stirred for overnight at 100° C. under O₂ atmosphere. The resulting mixture was concentrated under vacuum. The residue was purified by silica gel column chromatography, eluted with CH₂Cl₂/PE (1:1) to afford 3-bromo-6-methyl-2-(trifluoromethoxy)pyridine (200 mg, 37%) as a colorless oil. MS m/z: 256 [M+H]⁺.

Step 2: tert-butyl 2-(6-methyl-2-(trifluoromethoxy)pyridin-3-yl)-3-oxo-2,8-diazaspiro[4.5]decane-8-carboxylate: Followed the general procedure Y using 3-bromo-6-methyl-2-(trifluoromethoxy)pyridine (200, 0.784 mmol, 1.0 equiv.) and tert-butyl 3-oxo-2,8-diazaspiro[4.5]decane-8-carboxylate (219 mg, 0.862 mmol, 1.1 equiv.) as the starting materials, Ephos Pd G4 (36 mg, 39 μmol, 0.05 equiv.) as the catalyst to give tert-butyl 2-(6-methyl-2-(trifluoromethoxy)pyridin-3-yl)-3-oxo-2,8-diazaspiro[4.5]decane-8-carboxylate (250 mg, 74%) as a colorless oil. MS m/z: 430 [M+H]⁺.

Step 3: 2-(6-methyl-2-(trifluoromethoxy)pyridin-3-yl)-2,8-diazaspiro[4.5]decan-3-one hydrochloride: Followed the general procedure B using give tert-butyl 2-(6-methyl-2-(trifluoromethoxy)pyridin-3-yl)-3-oxo-2,8-diazaspiro[4.5]decane-8-carboxylate (100 mg) as the starting material to give the crude product 2-(6-methyl-2-(trifluoromethoxy)pyridin-3-yl)-2,8-diazaspiro[4.5]decan-3-one hydrochloride (80 mg). MS m/z: 330 [M+H]⁺

Step 4: 8-(1-(2,2-difluoroethyl)-1H-pyrazolo[3,4-b]pyrazin-6-yl)-2-(6-methyl-2-(trifluoromethoxy)pyridin-3-yl)-2,8-diazaspiro[4.5]decan-3-one: Followed the general procedure I using 2-(6-methyl-2-(trifluoromethoxy)pyridin-3-yl)-2,8-diazaspiro[4.5]decan-3-one hydrochloride (80 mg, 0.219 mmol, 1.0 equiv.) and 6-chloro-1-(2,2-difluoroethyl)-1H-pyrazolo[3,4-b]pyrazine (100 mg, 0.241 mmol, 1.0 equiv.) as the starting materials to give 8-(1-(2,2-difluoroethyl)-1H-pyrazolo[3,4-b]pyrazin-6-yl)-2-(6-methyl-2-(trifluoromethoxy)pyridin-3-yl)-2,8-diazaspiro[4.5]decan-3-one (61 mg, 54%) as a white solid. ¹H NMR (300 MHz, Chloroform-d) δ 8.27 (s, 1H), 8.05 (s, 1H), 7.70 (d, J=7.9 Hz, 1H), 7.12 (d, J=7.9 Hz, 1H), 6.22 (tt, J=55.6, 4.5 Hz, 1H), 4.66 (td, J=13.3, 4.4 Hz, 2H), 3.98-3.85 (m, 2H), 3.84-3.70 (m, 4H), 2.54 (d, J=12.7 Hz, 5H), 1.89 (t, J=5.6 Hz, 4H). MS m/z: 512.2 [M+H]⁺.

### Example 783: 8-(1-(2,2-difluoroethyl)-1H-pyrazolo[3,4-b]pyrazin-6-yl)-2-(6-methoxy-5-(trifluoromethyl)pyridin-2-yl)-2,8-diazaspiro[4.5]decan-1-one; Example 784: 8-(1-(2,2-difluoroethyl)-1H-pyrazolo[3,4-b]pyrazin-6-yl)-2-(6-methoxy-3-(trifluoromethyl)pyridin-2-yl)-2,8-diazaspiro[4.5]decan-1-one

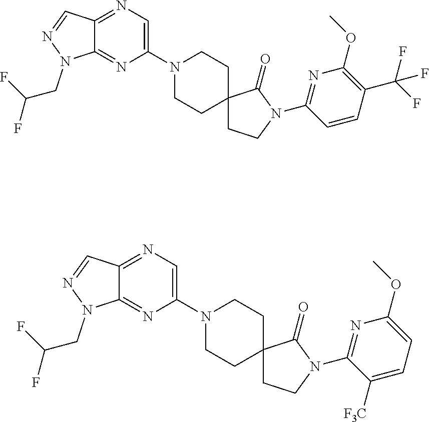 [SMILES: COc1nc(N2CCC3(CCN(c4cnc5cnn(CC(F)F)c5n4)CC3)C2=O)ccc1C(F)(F)F ; COc1ccc(C(F)(F)F)c(N2CCC3(CCN(c4cnc5cnn(CC(F)F)c5n4)CC3)C2=O)n1]

Step 1: 8-(1-(2,2-difluoroethyl)-1H-pyrazolo[3,4-b]pyrazin-6-yl)-2-(6-methoxy-5-(trifluoromethyl)pyridin-2-yl)-2,8-diazaspiro[4.5]decan-1-one: Followed the general procedure I using 2-(6-methoxy-5-(trifluoromethyl)pyridin-2-yl)-2,8-diazaspiro[4.5]decan-1-one hydrochloride (80 mg, 0.243 mmol, 1.0 equiv.) and 6-chloro-1-(2,2-difluoroethyl)-1H-pyrazolo[3,4-b]pyrazine (54 mg, 0.243 mmol, 1.0 equiv.) as the starting materials to give 8-(1-(2,2-difluoroethyl)-1H-pyrazolo[3,4-b]pyrazin-6-yl)-2-(6-methoxy-5-(trifluoromethyl)pyridin-2-yl)-2,8-diazaspiro[4.5]decan-1-one (40.8 mg, 32%) as a white solid and 8-(1-(2,2-difluoroethyl)-1H-pyrazolo[3,4-b]pyrazin-6-yl)-2-(6-methoxy-3-(trifluoromethyl)pyridin-2-yl)-2,8-diazaspiro[4.5]decan-1-one (45.9 mg, 36%) as a white solid.

### 8-(1-(2,2-difluoroethyl)-1H-pyrazolo[3,4-b]pyrazin-6-yl)-2-(6-methoxy-5-(trifluoromethyl)pyridin-2-yl)-2,8-diazaspiro[4.5]decan-1-one

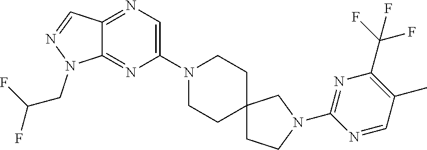 [SMILES: Cc1cnc(N2CCC3(CCN(c4cnc5cnn(CC(F)F)c5n4)CC3)C2)nc1C(F)(F)F]

Step 1: 5-bromo-2-iodo-4-(trifluoromethyl)pyrimidine: A solution of 5-bromo-4-(trifluoromethyl)pyrimidin-2-amine (500 mg, 2.07 mmol, 1.0 equiv.), CH₂I₂ (5534 mg, 20.7 mmol, 10.0 equiv.) and tBuONO (426 mg, 4.13 mmol, 2.0 equiv.) in ACN (10 mL) was stirred for 2 h at 70° C. under air atmosphere. Desired product could be detected by LCMS. The resulting mixture was extracted with EtOAc (3×30 mL). The combined organic layers were washed with water (2×10 mL), dried over anhydrous Na₂SO₄. After filtration, the filtrate was concentrated under reduced pressure. The residue was purified by silica gel column chromatography, eluted with EtOAc/PE=1/1 to afford 5-bromo-2-iodo-4-(trifluoromethyl)pyrimidine (400 mg, 54.9%) as a light yellow oil. MS m/z: 352 [M+H]⁺.

Step 2: tert-butyl 2-(5-methyl-4-(trifluoromethyl)pyrimidin-2-yl)-2,8-diazaspiro[4.5]decane-8-carboxylate: A mixture of 5-bromo-2-iodo-4-(trifluoromethyl)pyrimidine (400 mg, 1.13 mmol, 1.0 equiv.), methylboronic acid (102 mg, 1.70 mmol, 1.5 equiv.), Pd(dppf)Cl₂ (82.9 mg, 0.113 mmol, 0.1 equiv.) and Cs₂CO₃ (739 mg, 2.27 mmol, 2.0 equiv.) in 1,4-dioxane (5 mL) was stirred for 2 h at 80° C. under argon atmosphere. To the above mixture was added tert-butyl 2,8-diazaspiro[4.5]decane-8-carboxylate (272 mg, 1.13 mmol, 1.0 equiv.), XPhos (54.0 mg, 0.113 mmol, 0.10 equiv.), XPhos Pd G3 (96.0 mg, 0.113 mmol, 0.1 equiv.) in portions at room temperature. The resulting mixture was stirred for additional 2 h at 100° C. Desired product could be detected by LCMS. The resulting mixture was extracted with EtOAc (3×20 mL). The combined organic layers were washed with water (2×10 mL), dried over anhydrous Na₂SO₄. After filtration, the filtrate was concentrated under reduced pressure. The residue was purified by silica gel column chromatography, eluted with EtOAc/PE=1/1 to afford tert-butyl 2-(5-methyl-4-(trifluoromethyl)pyrimidin-2-yl)-2,8-diazaspiro[4.5]decane-8-carboxylate (50.0 mg, 11.0%) as a light yellow oil. MS m/z: 401 [M+H]⁺.

Step 3: 2-(5-methyl-4-(trifluoromethyl)pyrimidin-2-yl)-2,8-diazaspiro[4.5]decane hydrochloride: Followed the general procedure B using tert-butyl 2-(5-methyl-4-(trifluoromethyl)pyrimidin-2-yl)-2,8-diazaspiro[4.5]decane-8-carboxylate (50.0 mg, 0.125 mmol, 1.0 equiv.) as the starting material to give the crude product 2-(5-methyl-4-(trifluoromethyl)pyrimidin-2-yl)-2,8-diazaspiro[4.5]decane hydrochloride (40.0 mg). MS m/z: 301 [M+H]⁺.

Step 4: 8-(1-(2,2-difluoroethyl)-1H-pyrazolo[3,4-b]pyrazin-6-yl)-2-(5-methyl-4-(trifluoromethyl)pyrimidin-2-yl)-2,8-diazaspiro[4.5]decane: Followed the general procedure I using 2-(5-methyl-4-(trifluoromethyl)pyrimidin-2-yl)-2,8-diazaspiro[4.5]decane hydrochloride (40.0 mg, 0.133 mmol, 1.0 equiv) and 6-chloro-1-(2,2-difluoroethyl)-1H-pyrazolo[3,4-b]pyrazine (29.1 mg, 0.133 mmol, 1.0 equiv) as the starting materials to give 8-(1-(2,2-difluoroethyl)-1H-pyrazolo[3,4-b]pyrazin-6-yl)-2-(5-methyl-4-(trifluoromethyl)pyrimidin-2-yl)-2,8-diazaspiro[4.5]decane (3.00 mg, 4.7%) as a white solid. ¹H NMR (400 MHz, Chloroform-d) δ 8.29 (s, 2H), 8.08 (d, J=23.7 Hz, 1H), 6.22 (tt, J=55.6, 4.5 Hz, 1H), 4.66 (td, J=13.3, 4.5 Hz, 2H), 3.93 (dt, J=13.7, 5.4 Hz, 2H), 3.70 (q, J=9.6, 8.3 Hz, 4H), 3.57 (s, 2H), 2.26 (q, J=2.1 Hz, 3H), 1.96 (t, J=7.1 Hz, 2H), 1.85-1.70 (m, 4H). MS m/z: 483.05 [M+H]⁺.

### Example 786: 8-(1-(2,2-difluoroethyl)-1H-pyrazolo[3,4-b]pyrazin-6-yl)-2-(3-methyl-5-(trifluoromethyl)pyrazin-2-yl)-2,8-diazaspiro[4.5]decane

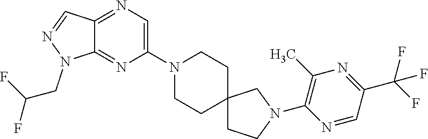 [SMILES: Cc1nc(C(F)(F)F)cnc1N1CCC2(CCN(c3cnc4cnn(CC(F)F)c4n3)CC2)C1]

Step 1: tert-butyl 2-(5-(trifluoromethyl)pyrazin-2-yl)-2,8-diazaspiro[4.5]decane-8-carboxylate: Followed the general procedure I using tert-butyl 2,8-diazaspiro[4.5]decane-8-carboxylate (300 mg, 1.25 mmol, 1.0 equiv), 2-chloro-5-(trifluoromethyl)pyrazine (251 mg, 1.37 mmol, 1.1 equiv) as the starting materials to give tert-butyl 2-(5-(trifluoromethyl)pyrazin-2-yl)-2,8-diazaspiro[4.5]decane-8-carboxylate (450 mg, 93.4%) as a white solid. MS m/z: 331 [M−tBu+H]⁺.

Step 2: tert-butyl 2-(3-chloro-5-(trifluoromethyl)pyrazin-2-yl)-2,8-diazaspiro[4.5]decane-8-carboxylate: Followed the general procedure V using tert-butyl 2-(5-(trifluoromethyl)pyrazin-2-yl)-2,8-diazaspiro[4.5]decane-8-carboxylate (300 mg, 0.775 mmol, 1.0 equiv) as the starting material, NCS (103 mg, 0.775 mmol, 1.0 equiv) as the reagent to give tert-butyl 2-(3-chloro-5-(trifluoromethyl)pyrazin-2-yl)-2,8-diazaspiro[4.5]decane-8-carboxylate (150 mg, 30.6%) as a white solid. MS m/z: 365 [M−tBu+H]⁺.

Step 3: tert-butyl 2-(3-methyl-5-(trifluoromethyl)pyrazin-2-yl)-2,8-diazaspiro[4.5]decane-8-carboxylate: Followed the general procedure L using tert-butyl 2-(3-chloro-5-(trifluoromethyl)pyrazin-2-yl)-2,8-diazaspiro[4.5]decane-8-carboxylate (150 mg, 0.356 mmol, 1.0 equiv) and methylboronic acid (21.3 mg, 0.356 mmol, 1.0 equiv) as the starting materials, Pd(PPh₃)₄ (41.2 mg, 0.036 mmol, 0.1 equiv) as the catalyst to give tert-butyl 2-(3-methyl-5-(trifluoromethyl)pyrazin-2-yl)-2,8-diazaspiro[4.5]decane-8-carboxylate (80.0 mg, 56.0%) as a white solid. MS m/z: 401 [M+H]⁺.

Step 4: 2-(3-methyl-5-(trifluoromethyl)pyrazin-2-yl)-2,8-diazaspiro[4.5]decane hydrochloride: Followed the general procedure B using tert-butyl 2-(3-methyl-5-(trifluoromethyl)pyrazin-2-yl)-2,8-diazaspiro[4.5]decane-8-carboxylate (80.0 mg, 0.200 mmol, 1.0 equiv) as the starting material to give the crude product 2-(3-methyl-5-(trifluoromethyl)pyrazin-2-yl)-2,8-diazaspiro[4.5]decane hydrochloride (50.0 mg). MS m/z: 301 [M+H]⁺

Step 5: 8-(1-(2,2-difluoroethyl)-1H-pyrazolo[3,4-b]pyrazin-6-yl)-2-(3-methyl-5-(trifluoromethyl)pyrazin-2-yl)-2,8-diazaspiro[4.5]decane: Followed the general procedure I using 2-(3-methyl-5-(trifluoromethyl)pyrazin-2-yl)-2,8-diazaspiro[4.5]decane hydrochloride (50.0 mg, 0.148 mmol, 1.0 equiv) and 6-chloro-1-(2,2-difluoroethyl)-1H-pyrazolo[3,4-b]pyrazine (32.5 mg, 0.148 mmol, 1.0 equiv) as the starting materials to give 8-(1-(2,2-difluoroethyl)-1H-pyrazolo[3,4-b]pyrazin-6-yl)-2-(3-methyl-5-(trifluoromethyl)pyrazin-2-yl)-2,8-diazaspiro[4.5]decane (40.7 mg, 56.0%) as a white solid. ¹H NMR (400 MHz, DMSO-d₆) δ 8.49 (s, 1H), 8.30 (s, 1H), 8.13 (s, 1H), 6.44 (tt, 3.8 Hz, 1H), 4.69 (td, 3.9 Hz, 2H), 3.82 (dt, 6H), 3.61 (s, 2H), 2.66 (s, 3H), 1.91 (t, 2H), 1.73-1.60 (m, 4H). MS m/z: 483.30 [M+H]⁺.

### Example 787: 8-(1-(2,2-difluoroethyl)-1H-pyrazolo[3,4-b]pyrazin-6-yl)-2-(4-methoxy-2-(trifluoromethyl)pyrimidin-5-yl)-2,8-diazaspiro[4.5]decane

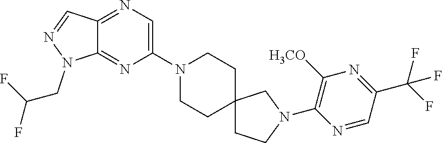 [SMILES: COc1nc(C(F)(F)F)cnc1N1CCC2(CCN(c3cnc4cnn(CC(F)F)c4n3)CC2)C1]

Step 1: tert-butyl 2-(2-(trifluoromethyl)pyrimidin-5-yl)-2,8-diazaspiro[4.5]decane-8-carboxylate: Followed the general procedure Y using tert-butyl 2,8-diazaspiro[4.5]decane-8-carboxylate (500 mg, 2.07 mmol, 1.0 equiv.) and 5-bromo-2-(trifluoromethyl)pyrimidine (470 mg, 2.07 mmol, 1.0 equiv.) as the starting materials, XPhos Pd G3 (175 mg, 0.207 mmol, 0.1 equiv.) as the catalyst to give tert-butyl 2-(2-(trifluoromethyl)pyrimidin-5-yl)-2,8-diazaspiro[4.5]decane-8-carboxylate (360 mg, 44.8%) as a white solid. MS m/z: 331 [M−tBu+H]⁺.

Step 2: tert-butyl 2-(4-bromo-2-(trifluoromethyl)pyrimidin-5-yl)-2,8-diazaspiro[4.5]decane-8-carboxylate: Followed the general procedure V using 2-(2-(trifluoromethyl)pyrimidin-5-yl)-2,8-diazaspiro[4.5]decane-8-carboxylate (360 mg, 0.930 mmol, 1.0 equiv.) as the starting material, NBS (331 mg, 1.86 mmol, 2.0 equiv.) as the reagent to give tert-butyl 2-(4-bromo-2-(trifluoromethyl)pyrimidin-5-yl)-2,8-diazaspiro[4.5]decane-8-carboxylate (120 mg, 27.2%) as a white solid. MS m/z: 409 [M−tBu+H]⁺.

Step 3: tert-butyl 2-(4-methoxy-2-(trifluoromethyl)pyrimidin-5-yl)-2,8-diazaspiro[4.5]decane-8-carboxylate: To a stirred solution of tert-butyl 2-(4-bromo-2-(trifluoromethyl)pyrimidin-5-yl)-2,8-diazaspiro[4.5]decane-8-carboxylate (120 mg, 0.258 mmol, 1.0 equiv) in MeOH (1.00 mL) was added MeONa (41.8 mg, 0.774 mmol, 3.0 equiv). The resulting mixture was stirred for 2 hours at 50° C. The resulting mixture was concentrated under reduced pressure. The residue was purified by silica gel column chromatography, eluted with EtOAc/PE (1/1) to afford tert-butyl 2-(4-methoxy-2-(trifluoromethyl)pyrimidin-5-yl)-2,8-diazaspiro[4.5]decane-8-carboxylate (50.0 mg, 46.5%) as a white solid. MS m/z: 417 [M−tBu+H]⁺.

Step 4: 2-(4-methoxy-2-(trifluoromethyl)pyrimidin-5-yl)-2,8-diazaspiro[4.5]decane hydrochloride: Followed the general procedure B using tert-butyl 2-(4-methoxy-2-(trifluoromethyl)pyrimidin-5-yl)-2,8-diazaspiro[4.5]decane-8-carboxylate (50.0 mg, 0.120 mmol, 1.0 equiv) as the starting material to give the crude product 2-(4-methoxy-2-(trifluoromethyl)pyrimidin-5-yl)-2,8-diazaspiro[4.5]decane hydrochloride (35.0 mg). MS m/z: 317 [M+H]⁺.

Step 5: 8-(1-(2,2-difluoroethyl)-1H-pyrazolo[3,4-b]pyrazin-6-yl)-2-(4-methoxy-2-(trifluoromethyl)pyrimidin-5-yl)-2,8-diazaspiro[4.5]decane: Followed the general procedure I using 2-(4-methoxy-2-(trifluoromethyl)pyrimidin-5-yl)-2,8-diazaspiro[4.5]decane hydrochloride (35.0 mg, 0.099 mmol, 1.00 equiv.) and 6-chloro-1-(2,2-difluoroethyl)-1H-pyrazolo[3,4-b]pyrazine (21.7 mg, 0.099 mmol, 1.00 equiv.) as the starting materials to give 8-(1-(2,2-difluoroethyl)-1H-pyrazolo[3,4-b]pyrazin-6-yl)-2-(4-methoxy-2-(trifluoromethyl)pyrimidin-5-yl)-2,8-diazaspiro[4.5]decane (10.0 mg, 20.2%) as a white solid. ¹H NMR (400 MHz, DMSO-d₆) δ 8.48 (s, 1H), 8.13 (s, 1H), 7.84 (s, 1H), 6.59-6.29 (m, 1H), 4.74-4.65 (m, 2H), 3.96 (s, 3H), 3.81 (t, 4H), 3.61 (t, 2H), 3.43 (s, 2H), 1.89 (t, 2H), 1.70-1.64 (m, 4H). MS m/z: 499.15 [M+H]⁺.

### Example 788: 8-(1-(2,2-difluoroethyl)-1H-pyrazolo[3,4-b]pyrazin-6-yl)-2-(2-methoxy-4-(trifluoromethyl)pyrimidin-5-yl)-2,8-diazaspiro[4.5]decane

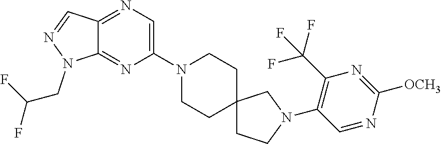 [SMILES: COc1ncc(N2CCC3(CCN(c4cnc5cnn(CC(F)F)c5n4)CC3)C2)c(C(F)(F)F)n1]

Step 1: tert-butyl 2-(2-(methylthio)-4-(trifluoromethyl)pyrimidin-5-yl)-2,8-diazaspiro[4.5]decane-8-carboxylate: Followed the general procedure Y using tert-butyl 2,8-diazaspiro[4.5]decane-8-carboxylate (264 mg, 1.09 mmol, 1.0 equiv.) and 5-chloro-2-(methylthio)-4-(trifluoromethyl)pyrimidine (250 mg, 1.09 mmol, 1.0 equiv.) as the starting materials, Ruphos Pd G3 (92 mg, 0.109 mmol, 0.1 equiv.) as the catalyst to give tert 2-(2-(methylthio)-4-(trifluoromethyl)pyrimidin-5-yl)-2,8-diazaspiro[4.5]decane-8-carboxylate (180 mg, 38%) as a white solid. MS m/z: 433 [M+H]⁺.

Step 2: tert-butyl 2-(2-(methylsulfonyl)-4-(trifluoromethyl)pyrimidin-5-yl)-2,8-diazaspiro[4.5]decane-8-carboxylate: To a stirred mixture of tert-butyl 2-(2-(methylthio)-4-(trifluoromethyl)pyrimidin-5-yl)-2,8-diazaspiro[4.5]decane-8-carboxylate (180 mg, 0.416 mmol, 1.0 equiv.) in MeOH (2 mL) was added Oxone (349.92 mg, 2.080 mmol, 5.0 equiv.) in portions at room temperature under air atmosphere. The resulting mixture was stirred for 2 h at room temperature under air atmosphere. The reaction was monitored by LCMS. Desired product could be detected by LCMS. The residue was purified by silica gel column chromatography, eluted with PE/EA (1:1) to afford tert-butyl 2-(2-(methylsulfonyl)-4-(trifluoromethyl)pyrimidin-5-yl)-2,8-diazaspiro[4.5]decane-8-carboxylate (160 mg, 83%) as a white solid. MS m/z: 433 [M+H]⁺.

Step 3: tert-butyl 2-(2-methoxy-4-(trifluoromethyl)pyrimidin-5-yl)-2,8-diazaspiro[4.5]decane-8-carboxylate: To a stirred solution of tert-butyl 2-(2-(methylsulfonyl)-4-(trifluoromethyl)pyrimidin-5-yl)-2,8-diazaspiro[4.5]decane-8-carboxylate (160 mg, 344 μmol, 1.0 equiv.) in MeOH (4 mL) was added NaOMe (20 mg, 344 μmol, 1.0 equiv.) dropwise at 60° C. under argon atmosphere. The mixture was allowed to cool down to room temperature. The reaction was monitored by LCMS. Desired product could be detected by LCMS. The residue was purified by reversed-phase flash chromatography with the following conditions: column, C18 silica gel; mobile phase, MeCN in Water (0.1% FA), 5% to 100% gradient in 20 min; detector, UV 254 nm. This resulted in tert-butyl 2-(2-methoxy-4-(trifluoromethyl)pyrimidin-5-yl)-2,8-diazaspiro[4.5]decane-8-carboxylate (120 mg, 837%) as a off-white oil. MS m/z: 417 [M+H]⁺.

Step 4: 2-(2-methoxy-4-(trifluoromethyl)pyrimidin-5-yl)-2,8-diazaspiro[4.5]decane: Followed the general procedure H using tert-butyl 2-(2-methoxy-4-(trifluoromethyl)pyrimidin-5-yl)-2,8-diazaspiro[4.5]decane-8-carboxylate (120 mg) as the starting material to give the crude product 2-(2-methoxy-4-(trifluoromethyl)pyrimidin-5-yl)-2,8-diazaspiro[4.5]decane (100 mg). MS m/z: 317 [M+H]⁺.

Step 5: 8-(1-(2,2-difluoroethyl)-1H-pyrazolo[3,4-b]pyrazin-6-yl)-2-(2-methoxy-4-(trifluoromethyl)pyrimidin-5-yl)-2,8-diazaspiro[4.5]decane: Followed the general procedure I using 2-(2-methoxy-4-(trifluoromethyl)pyrimidin-5-yl)-2,8-diazaspiro[4.5]decane (100 mg, 315 μmol, 1.0 equiv.) and 6-chloro-1-(2,2-difluoroethyl)-1H-pyrazolo[3,4-b]pyrazine (76 mg, 347 μmol, 1.1 equiv.) as the starting materials to give 8-(1-(2,2-difluoroethyl)-1H-pyrazolo[3,4-b]pyrazin-6-yl)-2-(2-methoxy-4-(trifluoromethyl)pyrimidin-5-yl)-2,8-diazaspiro[4.5]decane (120 mg, 75%) as a white solid. ¹H NMR (400 MHz, DMSO-d₆) δ 8.69 (s, 1H), 8.49 (s, 1H), 8.13 (s, 1H), 6.44 (tt, J=54.9, 3.8 Hz, 1H), 4.69 (td, J=15.0, 3.8 Hz, 2H), 3.89 (s, 3H), 3.80 (t, J=5.7 Hz, 4H), 3.42 (s, 2H), 3.22 (s, 2H), 1.90 (t, J=7.0 Hz, 2H), 1.67 (q, J=4.7 Hz, 4H). MS m/z: 499.15 [M+H]⁺.

### Example 789: 2-(1-(2,2-difluoroethyl)-1H-pyrazolo[3,4-b]pyrazin-6-yl)-8-(6-(trifluoromethyl) pyridin-3-yl)-2,8-diazaspiro[4.5]decan-7-one

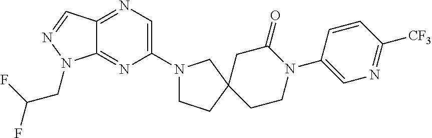 [SMILES: O=C1CC2(CCN(c3cnc4cnn(CC(F)F)c4n3)C2)CCN1c1ccc(C(F)(F)F)nc1]

Step 1: tert-butyl 7-oxo-8-(6-(trifluoromethyl)pyridin-3-yl)-2,8-diazaspiro[4.5]decane-2-carboxylate: Followed the general procedure Y using tert-butyl 7-oxo-2,8-diazaspiro[4.5]decane-2-carboxylate (100 mg, 0.39 mmol, 1.0 equiv.) and 5-bromo-2-(trifluoromethyl)pyridine (106 mg, 0.47 mmol, 1.2 equiv.) as the starting materials, EPhos Pd G4 (36 mg, 0.04 mmol, 0.1 equiv.) as the catalyst to give tert-butyl 7-oxo-8-(4-(trifluoromethyl)pyridin-2-yl)-2,8-diazaspiro[4.5]decane-2-carboxylate (70 mg, 44%) as a colorless oil. MS m/z: 400 [M+H]⁺.

Step 2: 8-(6-(trifluoromethyl)pyridin-3-yl)-2,8-diazaspiro[4.5]decan-7-one hydrochloride: Followed the general procedure B using tert-butyl 7-oxo-8-(6-(trifluoromethyl)pyridin-3-yl)-2,8-diazaspiro[4.5]decane-2-carboxylate (50 mg) as the starting material to give the crude product 8-(6-(trifluoromethyl)pyridin-3-yl)-2,8-diazaspiro[4.5]decan-7-one hydrochloride (50 mg). MS m/z: 300 [M+H]⁺.

Step 3: 2-(1-(2,2-difluoroethyl)-1H-pyrazolo[3,4-b]pyrazin-6-yl)-8-(6-(trifluoromethyl)pyridin-3-yl)-2,8-diazaspiro[4.5]decan-7-one: Followed the general procedure I using 8-(6-(trifluoromethyl)pyridin-3-yl)-2,8-diazaspiro[4.5]decan-7-one hydrochloride (20 mg, 0.06 mmol, 1.0 equiv.) and 6-chloro-1-(2,2-difluoroethyl)-1H-pyrazolo[3,4-b]pyrazine (15 mg, 0.07 mmol, 1.2 equiv.) as the starting materials to give 2-(1-(2,2-difluoroethyl)-1H-pyrazolo[3,4-b]pyrazin-6-yl)-8-(6-(trifluoromethyl)pyridin-3-yl)-2,8-diazaspiro[4.5]decan-7-one (9 mg, 30%) as a white solid. ¹H NMR (400 MHz, Methanol-d₄) δ 8.69 (d, J=2.4 Hz, 1H), 7.97 (d, J=8.4 Hz, 2H), 7.91 (s, 1H), 7.79 (d, J=8.3 Hz, 1H), 6.18 (tt, J=55.4, 4.2 Hz, 1H), 4.60 (td, J=14.1, 4.2 Hz, 2H), 3.87-3.80 (m, 2H), 3.74 (td, J=7.2, 6.5, 4.5 Hz, 2H), 3.60 (s, 2H), 2.62 (d, J=7.0 Hz, 2H), 2.13-2.02 (m, 4H). MS m/z: 482.15 [M+H]⁺.

### Example 790: 2-(1-(2,2-difluoroethyl)-1H-pyrazolo[3,4-b]pyrazin-6-yl)-7-(6-(trifluoromethyl)pyridin-3-yl)-2,7-diazaspiro[4.5]decan-8-one

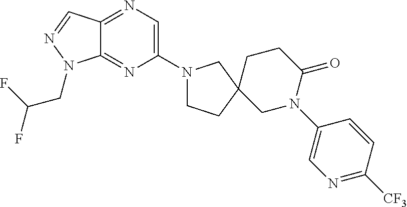 [SMILES: O=C1CCC2(CCN(c3cnc4cnn(CC(F)F)c4n3)C2)CN1c1ccc(C(F)(F)F)nc1]

Step 1: 7-(6-(trifluoromethyl)pyridin-3-yl)-2,7-diazaspiro[4.5]decan-8-one hydrochloride: Followed the general procedure B using tert-butyl 8-oxo-7-(6-(trifluoromethyl)pyridin-3-yl)-2,7-diazaspiro[4.5]decane-2-carboxylate (50 mg) as the starting material to give the crude product 7-(6-(trifluoromethyl)pyridin-3-yl)-2,7-diazaspiro[4.5]decan-8-one hydrochloride (50 mg). MS m/z: 300 [M+H]⁺

Step 2: 2-(1-(2,2-difluoroethyl)-1H-pyrazolo[3,4-b]pyrazin-6-yl)-7-(6-(trifluoromethyl)pyridin-3-yl)-2,7-diazaspiro[4.5]decan-8-one: Followed the general procedure I using 7-[6-(trifluoromethyl)pyridin-3-yl]-2,7-diazaspiro[4.5]decan-8-one hydrochloride (20 mg, 0.06 mmol, 1.0 equiv.) and 6-chloro-1-(2,2-difluoroethyl)-1H-pyrazolo[3,4-b]pyrazine (15 mg, 0.072 mmol, 1.2 equiv.) as the starting materials to give 2-(1-(2,2-difluoroethyl)-1H-pyrazolo[3,4-b]pyrazin-6-yl)-7-(6-(trifluoromethyl)pyridin-3-yl)-2,7-diazaspiro[4.5]decan-8-one (6.3 mg, 21%) as a white solid. ¹H NMR (400 MHz, Methanol-d₄) δ 8.69 (d, J=2.4 Hz, 1H), 7.97 (d, J=8.4 Hz, 2H), 7.91 (s, 1H), 7.79 (d, J=8.3 Hz, 1H), 6.18 (tt, J=55.4, 4.2 Hz, 1H), 4.60 (td, J=14.1, 4.2 Hz, 2H), 3.88-3.67 (m, 5H), 2.62 (d, J=7.0 Hz, 2H), 2.15-1.99 (m, 5H). MS m/z: 482.2 [M+H]⁺.

### Example 791: 2-(1-(2,2-difluoroethyl)-1H-pyrazolo[3,4-b]pyrazin-6-yl)-8-(5-(trifluoromethyl)pyridin-2-yl)-2,8-diazaspiro[4.5]decan-7-one

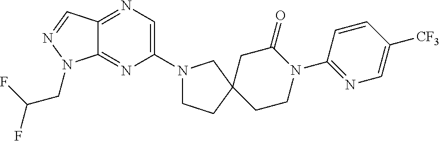 [SMILES: O=C1CC2(CCN(c3cnc4cnn(CC(F)F)c4n3)C2)CCN1c1ccc(C(F)(F)F)cn1]

Step 1: tert-butyl 7-oxo-8-(6-(trifluoromethyl)pyridin-3-yl)-2,8-diazaspiro[4.5]decane-2-carboxylate: Followed the general procedure Y using tert-butyl 7-oxo-2,8-diazaspiro[4.5]decane-2-carboxylate (150 mg, 0.59 mmol, 1.0 equiv.) and 2-bromo-5-(trifluoromethyl)pyridine (133 mg, 0.59 mmol, 1.0 equiv.) as the starting materials EPhos Pd G4 (36 mg, 0.06 mmol, 0.1 equiv.) to give tert-butyl 7-oxo-8-(5-(trifluoromethyl)pyridin-2-yl)-2,8-diazaspiro[4.5]decane-2-carboxylate (70 mg, 44%) as a colorless oil. MS m/z: 400 [M+H]⁺.

Step 2: 8-(5-(trifluoromethyl)pyridin-2-yl)-2,8-diazaspiro[4.5]decan-7-one hydrochloride: Followed the general procedure B using tert-butyl 7-oxo-8-(6-(trifluoromethyl)pyridin-2-yl)-2,8-diazaspiro[4.5]decane-2-carboxylate (40 mg) as the starting material to give the crude product 8-(5-(trifluoromethyl)pyridin-2-yl)-2,8-diazaspiro[4.5]decan-7-one hydrochloride (50 mg). MS m/z: 300 [M+H]⁺.

Step 3: 2-(1-(2,2-difluoroethyl)-1H-pyrazolo[3,4-b]pyrazin-6-yl)-8-(5-(trifluoromethyl)pyridin-2-yl)-2,8-diazaspiro[4.5]decan-7-one: Followed the general procedure I using 8-(5-(trifluoromethyl)pyridin-2-yl)-2,8-diazaspiro[4.5]decan-7-one hydrochloride (20 mg, 0.06 mmol, 1.0 equiv.) and 6-chloro-1-(2,2-difluoroethyl)-1H-pyrazolo[3,4-b]pyrazine (15 mg, 0.07 mmol, 1.2 equiv.) as the starting materials to give 2-(1-(2,2-difluoroethyl)-1H-pyrazolo[3,4-b]pyrazin-6-yl)-8-(5-(trifluoromethyl)pyridin-2-yl)-2,8-diazaspiro[4.5]decan-7-one (9 mg, 30%) as a white solid. ¹H NMR (300 MHz, DMSO-d₆) δ 8.86 (s, 1H), 8.21 (d, J=8.0 Hz, 1H), 8.12 (dd, J=7.3, 3.2 Hz, 3H), 6.45 (t, J=55.0 Hz, 1H), 4.77-4.60 (m, 2H), 4.10-4.01 (m, 2H), 3.73 (s, 2H), 3.58 (s, 2H), 2.71 (d, J=6.4 Hz, 2H), 2.06 (d, J=6.2 Hz, 4H). MS m/z: 482.15 [M+H]⁺.

### Example 792: 2-(1-(2,2-difluoroethyl)-1H-pyrazolo[3,4-b]pyrazin-6-yl)-7-(5-(trifluoromethyl)pyridin-2-yl)-2,7-diazaspiro[4.5]decan-8-one

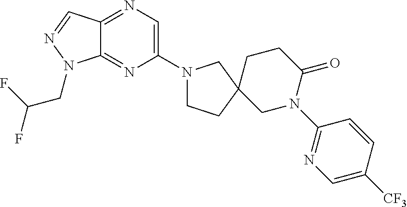 [SMILES: O=C1CCC2(CCN(c3cnc4cnn(CC(F)F)c4n3)C2)CN1c1ccc(C(F)(F)F)cn1]

Step 1: 7-(5-(trifluoromethyl)pyridin-2-yl)-2,7-diazaspiro[4.5]decan-8-one hydrochloride: Followed the general procedure B using tert-butyl 7-oxo-8-(6-(trifluoromethyl)pyridin-3-yl)-2,8-diazaspiro[4.5]decane-2-carboxylate (35 mg, 0.161 mmol, 1.0 equiv.) as the starting material to give the crude product 7-(5-(trifluoromethyl)pyridin-2-yl)-2,7-diazaspiro[4.5]decan-8-one hydrochloride (40 mg). MS m/z: 300 [M+H]⁺.

Step 2: 2-(1-(2,2-difluoroethyl)-1H-pyrazolo[3,4-b]pyrazin-6-yl)-7-(5-(trifluoromethyl)pyridin-2-yl)-2,7-diazaspiro[4.5]decan-8-one: Followed the general procedure I using 7-(5-(trifluoromethyl)pyridin-2-yl)-2,7-diazaspiro[4.5]decan-8-one hydrochloride (40 mg, 0.067 mmol, 1.00 equiv.) and 6-chloro-1-(2,2-difluoroethyl)-1H-pyrazolo[3,4-b]pyrazine (35 mg, 0.08 mmol, 1.2 equiv.) as the starting materials to give 2-(1-(2,2-difluoroethyl)-1H-pyrazolo[3,4-b]pyrazin-6-yl)-7-(5-(trifluoromethyl)pyridin-2-yl)-2,7-diazaspiro[4.5]decan-8-one (26.8 mg, 40%) as a white solid. ¹H NMR (300 MHz, DMSO-d₆) δ 8.77 (s, 1H), 8.24-8.01 (m, 4H), 6.66-6.19 (m, 1H), 4.67 (td, J=14.9, 3.8 Hz, 2H), 3.98 (s, 2H), 3.73 (d, J=11.5 Hz, 3H), 3.55 (d, J=11.3 Hz, 1H), 2.69 (t, J=7.0 Hz, 2H), 2.18-1.91 (m, 4H). MS m/z: 482.15 [M+H]⁺.

### Example 793: 2-(1-(2,2-difluoroethyl)-1H-pyrazolo[3,4-b]pyrazin-6-yl)-8-(4-(trifluoromethyl) pyridin-2-yl)-2,8-diazaspiro[4.5]decan-7-one

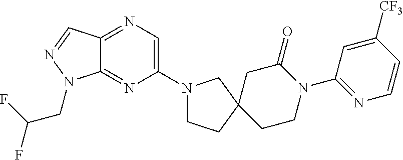 [SMILES: O=C1CC2(CCN(c3cnc4cnn(CC(F)F)c4n3)C2)CCN1c1cc(C(F)(F)F)ccn1]

Step 1: tert-butyl (Z)-7-(hydroxyimino)-2-azaspiro[4.4]nonane-2-carboxylate: To a stirred mixture of tert-butyl 7-oxo-2-azaspiro[4.4]nonane-2-carboxylate (500 mg, 2.08 mmol, 1.0 equiv) and hydroxylamine hydrochloride (174 mg, 2.50 mmol, 1.2 equiv) in EtOH:H₂O=10:1 (5 mL) was added K₂CO₃ (577 mg, 4.17 mmol, 2.0 equiv) in portions at room temperature under air atmosphere. The resulting mixture was stirred for overnight at 80° C. under inert atmosphere. The resulting mixture was purified by reverse phase flash with the following conditions (column, C18 silica gel; mobile phase, MeCN in water, 30% to 60% gradient in 15 min; detector, UV 220 nm.) to afford tert-butyl (Z)-7-(hydroxyimino)-2-azaspiro[4.4]nonane-2-carboxylate (500 mg, 94%) as a colorless oil.

Step 2: tert-butyl 7-oxo-2,8-diazaspiro[4.5]decane-2-carboxylate: To a stirred solution of tert-butyl (Z)-7-(hydroxyimino)-2-azaspiro[4.4]nonane-2-carboxylate (200 mg, 0.78 mmol, 1.0 equiv) and phosphorus oxychloride (300 mg, 1.95 mmol, 2.5 equiv) in 1,4-dioxane in portions at room temperature under air atmosphere. The mixture was neutralized to pH 8 with saturated NaHCO₃(aq.). The residue product was purified by reverse phase flash with the following conditions (The residue was purified by reverse flash chromatography with the following conditions: column, C18 silica gel; mobile phase, MeCN in Water (0.1% FA), 20% to 60% gradient in 10 min; detector, UV220 nm.) to afford tert-butyl 7-oxo-2,8-diazaspiro[4.5]decane-2-carboxylate (70 mg, 35.0%) as a light brown solid.

Step 3: tert-butyl 7-oxo-8-(4-(trifluoromethyl)pyridin-2-yl)-2,8-diazaspiro[4.5]decane-2-carboxylate: Followed the general procedure Y using tert-butyl 7-oxo-2,8-diazaspiro[4.5]decane-2-carboxylate (100 mg, 0.39 mmol, 1.0 equiv.) and 2-bromo-4-(trifluoromethyl)pyridine (106 mg, 0.47 mmol, 1.2 equiv.) as the starting materials, EPhos Pd G4 (36 mg, 0.04 mmol, 0.1 equiv.) as the catalyst to give tert-butyl 7-oxo-8-(4-(trifluoromethyl)pyridin-2-yl)-2,8-diazaspiro[4.5]decane-2-carboxylate (70 mg, 44%) as a colorless oil. MS m/z: 400 [M+H]⁺.

Step 4: 8-(4-(trifluoromethyl)pyridin-2-yl)-2,8-diazaspiro[4.5]decan-7-one hydrochloride: Followed the general procedure B using tert-butyl 7-oxo-8-(4-(trifluoromethyl)pyridin-2-yl)-2,8-diazaspiro[4.5]decane-2-carboxylate (50 mg) as the starting material to give the crude product 8-(4-(trifluoromethyl)pyridin-2-yl)-2,8-diazaspiro[4.5]decan-7-one hydrochloride (50 mg). MS m/z: 300 [M+H]⁺.

Step 5: 2-(1-(2,2-difluoroethyl)-1H-pyrazolo[3,4-b]pyrazin-6-yl)-8-(4-(trifluoromethyl)pyridin-2-yl)-2,8-diazaspiro[4.5]decan-7-one: Followed the general procedure I using 8-(4-(trifluoromethyl)pyridin-2-yl)-2,8-diazaspiro[4.5]decan-7-one hydrochloride (20 mg, 0.06 mmol, 1.0 equiv.) and 6-chloro-1-(2,2-difluoroethyl)-1H-pyrazolo[3,4-b]pyrazine (15 mg, 0.07 mmol, 1.2 equiv.) as the starting materials to give 2-(1-(2,2-difluoroethyl)-1H-pyrazolo[3,4-b]pyrazin-6-yl)-8-(4-(trifluoromethyl)pyridin-2-yl)-2,8-diazaspiro[4.5]decan-7-one (9 mg, 30%) as a white solid. ¹H NMR (400 MHz, DMSO-d₆) δ 8.73 (s, 1H), 8.22 (s, 1H), 8.12 (d, J=7.0 Hz, 2H), 7.58 (s, 1H), 6.45 (t, J=55.0 Hz, 1H), 4.74-4.62 (m, 2H), 4.04 (s, 2H), 3.74 (s, 2H), 3.58 (s, 2H), 2.69 (d, J=8.8 Hz, 2H), 2.04 (s, 4H). MS m/z: 482.15 [M+H]⁺.

### Example 794: 2-(1-(2,2-difluoroethyl)-1H-pyrazolo[3,4-b]pyrazin-6-yl)-7-(4-(trifluoromethyl)pyridin-2-yl)-2,7-diazaspiro[4.5]decan-8-one

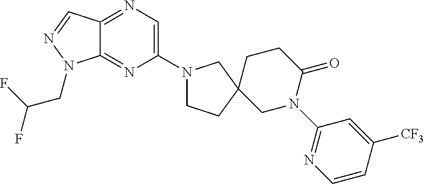 [SMILES: O=C1CCC2(CCN(c3cnc4cnn(CC(F)F)c4n3)C2)CN1c1cc(C(F)(F)F)ccn1]

Step 1: 7-(4-(trifluoromethyl)pyridin-2-yl)-2,7-diazaspiro[4.5]decan-8-one hydrochloride: Followed the general procedure B using tert-butyl 8-oxo-7-(4-(trifluoromethyl)pyridin-2-yl)-2,7-diazaspiro[4.5]decane-2-carboxylate (100 mg) as the starting material to give the crude product 7-(4-(trifluoromethyl)pyridin-2-yl)-2,7-diazaspiro[4.5]decan-8-one hydrochloride (50 mg). MS m/z: 300 [M+H]⁺.

Step 2: 2-(1-(2,2-difluoroethyl)-1H-pyrazolo[3,4-b]pyrazin-6-yl)-7-(4-(trifluoromethyl)pyridin-2-yl)-2,7-diazaspiro[4.5]decan-8-one: Followed the general procedure I using 7-(4-(trifluoromethyl)pyridin-2-yl)-2,7-diazaspiro[4.5]decan-8-one hydrochloride (20 mg, 0.06 mmol, 1.0 equiv.) and 6-chloro-1-(2,2-difluoroethyl)-1H-pyrazolo[3,4-b]pyrazine (15 mg, 0.07 mmol, 1.2 equiv.) as the starting materials to give 2-(1-(2,2-difluoroethyl)-1H-pyrazolo[3,4-b]pyrazin-6-yl)-7-(4-(trifluoromethyl)pyridin-2-yl)-2,7-diazaspiro[4.5]decan-8-one (9 mg, 30%) as a white solid. ¹H NMR (400 MHz, DMSO-d₆) δ 8.66 (s, 1H), 8.19 (s, 1H), 8.11 (d, J=7.2 Hz, 2H), 7.55 (s, 1H), 6.44 (t, J=56.0 Hz, 1H), 4.68 (t, J=15.6 Hz, 2H), 3.95 (s, 2H), 3.79-3.56 (m, 4H), 2.68 (t, J=6.2 Hz, 2H), 2.13 (s, 1H), 2.01 (s, 3H). 482.15 [M+H]⁺.

### Example 795: 2-(1-(2,2-difluoroethyl)-1H-pyrazolo[3,4-b]pyrazin-6-yl)-8-(5-(trifluoromethyl)pyridin-3-yl)-2,8-diazaspiro[4.5]decan-7-one

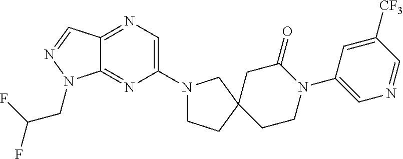 [SMILES: O=C1CC2(CCN(c3cnc4cnn(CC(F)F)c4n3)C2)CCN1c1cncc(C(F)(F)F)c1]

Step 1: tert-butyl 7-oxo-8-(5-(trifluoromethyl)pyridin-3-yl)-2,8-diazaspiro[4.5]decane-2-carboxylate: Followed the general procedure Y using tert-butyl 7-oxo-2,8-diazaspiro[4.5]decane-2-carboxylate (100 mg, 0.39 mmol, 1.0 equiv.) and 3-bromo-5-(trifluoromethyl)pyridine (106 mg, 0.47 mmol, 1.2 equiv.) as the starting materials EPhos Pd G4 (36 mg, 0.04 mmol, 0.1 equiv.) as the catalyst to give tert-butyl 7-oxo-8-(5-(trifluoromethyl)pyridin-3-yl)-2,8-diazaspiro[4.5]decane-2-carboxylate (50 mg, 32%) as a light brown solid. MS m/z: 400 [M+H]⁺.

Step 2: 8-(5-(trifluoromethyl)pyridin-3-yl)-2,8-diazaspiro[4.5]decan-7-one hydrochloride: Followed the general procedure B using tert-butyl 7-oxo-8-(5-(trifluoromethyl)pyridin-3-yl)-2,8-diazaspiro[4.5]decane-2-carboxylate (50 mg) as the starting material to give the crude product 8-(5-(trifluoromethyl)pyridin-3-yl)-2,8-diazaspiro[4.5]decan-7-one hydrochloride (50 mg). MS m/z: 300 [M+H]⁺.

Step 3: 2-(1-(2,2-difluoroethyl)-1H-pyrazolo[3,4-b]pyrazin-6-yl)-8-(4-(trifluoromethyl)pyridin-2-yl)-2,8-diazaspiro[4.5]decan-7-one: Followed the general procedure I using 8-(5-(trifluoromethyl)pyridin-3-yl)-2,8-diazaspiro[4.5]decan-7-one hydrochloride (50 mg, 0.17 mmol, 1.0 equiv.) and 6-chloro-1-(2,2-difluoroethyl)-1H-pyrazolo[3,4-b]pyrazine (44 mg, 0.20 mmol, 1.2 equiv.) as the starting materials to give 2-(1-(2,2-difluoroethyl)-1H-pyrazolo[3,4-b]pyrazin-6-yl)-8-(5-(trifluoromethyl)pyridin-3-yl)-2,8-diazaspiro[4.5]decan-7-one (24.4 mg, 30%) as a white solid. ¹H NMR (300 MHz, Chloroform-d) δ 8.73 (s, 1H), 8.09 (s, 1H), 8.01 (s, 1H), 7.80 (s, 1H), 7.60 (s, 1H), 6.23 (s, 1H), 4.75-4.61 (m, 2H), 3.95-3.75 (m, 4H), 3.65 (s, 2H), 2.75 (s, 2H), 2.16 (s, 4H). MS m/z: 482.1 [M+H]⁺.

### Example 796: 2-(1-(2,2-difluoroethyl)-1H-pyrazolo[3,4-b]pyrazin-6-yl)-7-(5-(trifluoromethyl) pyridin-3-yl)-2,7-diazaspiro[4.5]decan-8-one

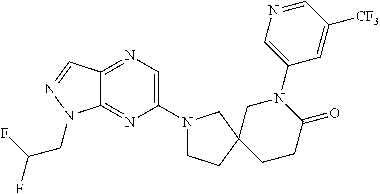 [SMILES: O=C1CCC2(CCN(c3cnc4cnn(CC(F)F)c4n3)C2)CN1c1cncc(C(F)(F)F)c1]

Step 1: 7-(5-(trifluoromethyl)pyridin-3-yl)-2,7-diazaspiro[4.5]decan-8-one hydrochloride: Followed the general procedure B using tert-butyl 8-oxo-7-(5-(trifluoromethyl)pyridin-3-yl)-2,7-diazaspiro[4.5]decane-2-carboxylate (40 mg) as the starting material to give the crude product 7-(5-(trifluoromethyl)pyridin-3-yl)-2,7-diazaspiro[4.5]decan-8-one hydrochloride (50 mg). MS m/z: 300 [M+H]⁺.

Step 2: 2-(1-(2,2-difluoroethyl)-1H-pyrazolo[3,4-b]pyrazin-6-yl)-7-(5-(trifluoromethyl)pyridin-3-yl)-2,7-diazaspiro[4.5]decan-8-one: Followed the general procedure I using 7-(5-(trifluoromethyl)pyridin-3-yl)-2,7-diazaspiro[4.5]decan-8-one hydrochloride (50 mg, 0.17 mmol, 1.0 equiv.) and 6-chloro-1-(2,2-difluoroethyl)-1H-pyrazolo[3,4-b]pyrazine (44 mg, 0.20 mmol, 1.2 equiv.) as the starting materials to give 2-(1-(2,2-difluoroethyl)-1H-pyrazolo[3,4-b]pyrazin-6-yl)-7-(5-(trifluoromethyl)pyridin-3-yl)-2,7-diazaspiro[4.5]decan-8-one (20.9 mg, 21%) as a white solid. ¹H NMR (300 MHz, Chloroform-d) δ 8.70 (d, J=5.4 Hz, 1H), 8.08 (s, 1H), 7.97 (s, 1H), 7.71 (d, J=2.0 Hz, 1H), 7.51 (d, J=5.5 Hz, 1H), 6.43-6.01 (m, 1H), 4.68 (td, J=13.3, 4.4 Hz, 2H), 3.86-3.59 (m, 6H), 2.80 (t, J=7.1 Hz, 2H), 2.18 (ddd, J=31.6, 12.3, 7.3 Hz, 4H). MS m/z: 482.1 [M+H]⁺.

### Example 797: 8-(1-(2,2-difluoroethyl)-1H-pyrazolo[3,4-b]pyrazin-6-yl)-1′-(6-(trifluoromethyl)pyridin-3-yl)-8-azaspiro[bicyclo[3.2.1]octane-3,3′-pyrrolidin]-2′-one

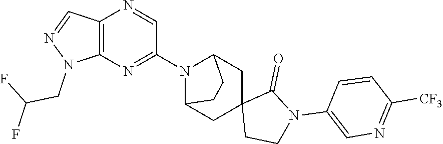 [SMILES: O=C1N(c2ccc(C(F)(F)F)nc2)CCC12CC1CCC(C2)N1c1cnc2cnn(CC(F)F)c2n1]

Step 1: tert-butyl 2′-oxo-1′-(6-(trifluoromethyl)pyridin-3-yl)-8-azaspiro[bicyclo[3.2.1]octane-3,3′-pyrrolidine]-8-carboxylate: Followed the general procedure E using tert-butyl 2′-oxo-8-azaspiro[bicyclo[3.2.1]octane-3,3′-pyrrolidine]-8-carboxylate (300 mg, 1.07 mmol, 1.0 equiv.) and 5-fluoro-2-(trifluoromethyl)pyridine (194 mg, 1.17 mmol, 1.1 equiv.) as the starting materials to give tert-butyl 2′-oxo-1′-(6-(trifluoromethyl)pyridin-3-yl)-8-azaspiro[bicyclo[3.2.1]octane-3,3′-pyrrolidine]-8-carboxylate (110 mg, 24%) as a colorless oil. MS m/z: 426 [M+H]⁺.

Step 2: 1′-(6-(trifluoromethyl)pyridin-3-yl)-8-azaspiro[bicyclo[3.2.1]octane-3,3′-pyrrolidin]-2′-one hydrochloride: Followed the general procedure B using tert-butyl 2′-oxo-1′-(6-(trifluoromethyl)pyridin-3-yl)-8-azaspiro[bicyclo[3.2.1]octane-3,3′-pyrrolidine]-8-carboxylate (110 mg) as the starting material to give the crude product 1′-(6-(trifluoromethyl)pyridin-3-yl)-8-azaspiro[bicyclo[3.2.1]octane-3,3′-pyrrolidin]-2′-one hydrochloride (90 mg). MS m/z: 326 [M+H]⁺.

Step 3: 8-(1-(2,2-difluoroethyl)-1H-pyrazolo[3,4-b]pyrazin-6-yl)-1′-(6-(trifluoromethyl)pyridin-3-yl)-8-azaspiro[bicyclo[3.2.1]octane-3,3′-pyrrolidin]-2′-one: Followed the general procedure I using 1′-(1-(2,2-difluoroethyl)-1H-pyrazolo[3,4-b]pyrazin-6-yl)-8-azaspiro[bicyclo[3.2.1]octane-3,3′-pyrrolidin]-2′-one hydrochloride (90 mg, 0.249 mmol, 1.0 equiv.) and 5-bromo-2-(trifluoromethyl)pyridine (60 mg, 0.274 mmol, 1.1 equiv.) as the starting materials to give 8-(1-(2,2-difluoroethyl)-1H-pyrazolo[3,4-b]pyrazin-6-yl)-1′-(6-(trifluoromethyl)pyridin-3-yl)-8-azaspiro[bicyclo[3.2.1]octane-3,3′-pyrrolidin]-2′-one (88 mg, 70%) as a white solid. ¹H NMR (400 MHz, DMSO-d₆) δ 9.05 (d, J=2.6 Hz, 1H), 8.45-8.38 (m, 1H), 8.36 (s, 1H), 8.14 (s, 1H), 7.96-7.88 (m, 1H), 6.45 (tt, J=54.9, 3.9 Hz, 1H), 4.89-4.80 (m, 2H), 4.71 (td, J=15.0, 3.8 Hz, 2H), 3.78 (t, J=6.4 Hz, 2H), 2.34-2.25 (m, 2H), 2.08-2.01 (m, 4H), 2.00-1.93 (m, 2H), 1.82 (t, J=6.4 Hz, 2H). MS m/z: 508.25 [M+H]⁺.

### Example 798: 8-(1-(2,2-difluoroethyl)-1H-pyrazolo[3,4-b]pyrazin-6-yl)-1′-(5-(trifluoromethyl)pyridin-2-yl)-8-azaspiro[bicyclo[3.2.1]octane-3,3′-pyrrolidin]-2′-one

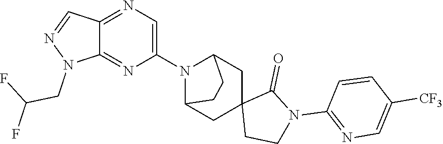 [SMILES: O=C1N(c2ccc(C(F)(F)F)cn2)CCC12CC1CCC(C2)N1c1cnc2cnn(CC(F)F)c2n1]

Step 1: tert-butyl 2′-oxo-1′-(5-(trifluoromethyl)pyridin-2-yl)-8-azaspiro[bicyclo[3.2.1]octane-3,3′-pyrrolidine]-8-carboxylate: Followed the general procedure E using tert-butyl 2′-oxo-8-azaspiro[bicyclo[3.2.1]octane-3,3′-pyrrolidine]-8-carboxylate (150 mg, 0.535 mmol, 1.0 equiv.) and 2-fluoro-5-(trifluoromethyl)pyridine (97 mg, 0.589 mmol, 1.1 equiv.) as the starting materials to give tert-butyl 2′-oxo-1′-(5-(trifluoromethyl)pyridin-2-yl)-8-azaspiro[bicyclo[3.2.1]octane-3,3′-pyrrolidine]-8-carboxylate (100 mg, 43%) as a colorless oil. MS m/z: 426 [M+H]⁺.

Step 2: 1′-(5-(trifluoromethyl)pyridin-2-yl)-8-azaspiro[bicyclo[3.2.1]octane-3,3′-pyrrolidin]-2′-one hydrochloride: Followed the general procedure B using tert-butyl 2′-oxo-1′-(5-(trifluoromethyl)pyridin-2-yl)-8-azaspiro[bicyclo[3.2.1]octane-3,3′-pyrrolidine]-8-carboxylate (100 mg) as the starting material to give the crude product 1′-(5-(trifluoromethyl)pyridin-2-yl)-8-azaspiro[bicyclo[3.2.1]octane-3,3′-pyrrolidin]-2′-one hydrochloride (90 mg). MS m/z: 326 [M+H]⁺.

Step 3: 2-(5-chloropyridin-3-yl)-8-(1-(2,2-difluoroethyl)-1H-pyrazolo[3,4-b]pyrazin-6-yl)-2,8-diazaspiro[4.5]decan-3-one: Followed the general procedure I using 1′-(5-(trifluoromethyl)pyridin-2-yl)-8-azaspiro[bicyclo[3.2.1]octane-3,3′-pyrrolidin]-2′-one hydrochloride (90 mg, 0.249 mmol, 1.0 equiv.) and 6-chloro-1-(2,2-difluoroethyl)-1H-pyrazolo[3,4-b]pyrazine (59 mg, 0.274 mmol, 1.2 equiv.) as the starting materials to give 8-(1-(2,2-difluoroethyl)-1H-pyrazolo[3,4-b]pyrazin-6-yl)-1′-(5-(trifluoromethyl)pyridin-2-yl)-8-azaspiro[bicyclo[3.2.1]octane-3,3′-pyrrolidin]-2′-one (44 mg, 34%) as a white solid. ¹H NMR (400 MHz, DMSO-d₆) δ 8.78 (s, 1H), 8.54 (d, J=8.9 Hz, 1H), 8.36 (s, 1H), 8.24-8.18 (m, 1H), 8.14 (s, 1H), 6.45 (tt, J=54.9, 3.8 Hz, 1H), 4.89-4.80 (m, 2H), 4.70 (td, J=15.0, 3.8 Hz, 2H), 3.86 (t, J=6.5 Hz, 2H), 2.36-2.22 (m, 2H), 2.12-2.02 (m, 4H), 2.02-1.93 (m, 2H), 1.77 (t, J=6.5 Hz, 2H). MS m/z: 508.25 [M+H]⁺.

### Example 799: 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.2.1]octane-8,3′-pyrrolidin]-2′-one

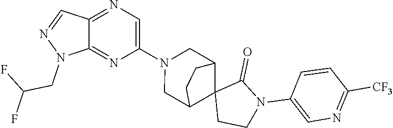 [SMILES: O=C1N(c2ccc(C(F)(F)F)nc2)CCC12C1CCC2CN(c2cnc3cnn(CC(F)F)c3n2)C1]

Step 1: 3-(tert-butyl) 8-methyl 8-(cyanomethyl)-3-azabicyclo[3.2.1]octane-3,8-dicarboxylate: Followed the general procedure AC using 3-(tert-butyl) 8-methyl 3-azabicyclo[3.2.1]octane-3,8-dicarboxylate (800 mg, 2.97 mmol, 1.0 equiv.) and 2-bromoacetonitrile (707 mg, 5.97 mmol, 2.0 equiv.) as the starting materials to give 3-(tert-butyl) 8-methyl 8-(cyanomethyl)-3-azabicyclo[3.2.1]octane-3,8-dicarboxylate (400 mg, 43%) as a colorless oil. MS m/z: 309 [M+H]⁺.

Step 2: tert-butyl 2′-oxo-3-azaspiro[bicyclo[3.2.1]octane-8,3′-pyrrolidine]-3-carboxylate: Followed the general procedure AB using 3-(tert-butyl) 8-methyl 8-(cyanomethyl)-3-azabicyclo[3.2.1]octane-3,8-dicarboxylate (400 mg, 1.29 mmol, 1.0 equiv.) as the starting material to give tert-butyl 2′-oxo-3-azaspiro[bicyclo[3.2.1]octane-8,3′-pyrrolidine]-3-carboxylate (250 mg, 68%) as a colorless oil. MS m/z: 281 [M+H]⁺.

Step 3: tert-butyl 2′-oxo-1′-(6-(trifluoromethyl)pyridin-3-yl)-3-azaspiro[bicyclo[3.2.1]octane-8,3′-pyrrolidine]-3-carboxylate: Followed the general procedure E using tert-butyl 2′-oxo-3-azaspiro[bicyclo[3.2.1]octane-8,3′-pyrrolidine]-3-carboxylate (50 mg, 0.178 mmol, 1.0 equiv.) and 5-fluoro-2-(trifluoromethyl)pyridine (44 mg, 0.268 mmol, 1.5 equiv.) as the starting materials to give tert-butyl 2′-oxo-1′-(6-(trifluoromethyl)pyridin-3-yl)-3-azaspiro[bicyclo[3.2.1]octane-8,3′-pyrrolidine]-3-carboxylate (30 mg, 40%) as a colorless oil. MS m/z: 426 [M+H]⁺.

Step 4: 1′-(6-(trifluoromethyl)pyridin-3-yl)-3-azaspiro[bicyclo[3.2.1]octane-8,3′-pyrrolidin]-2′-one hydrochloride: Followed the general procedure B using tert-butyl 2′-oxo-1′-(6-(trifluoromethyl)pyridin-3-yl)-3-azaspiro[bicyclo[3.2.1]octane-8,3′-pyrrolidine]-3-carboxylate (30 mg) as the starting material to give the crude product 1′-(6-(trifluoromethyl)pyridin-3-yl)-3-azaspiro[bicyclo[3.2.1]octane-8,3′-pyrrolidin]-2′-one hydrochloride (25 mg). MS m/z: 326 [M+H]⁺.

Step 5: 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.2.1]octane-8,3′-pyrrolidin]-2′-one: Followed the general procedure I using 1′-(6-(trifluoromethyl)pyridin-3-yl)-3-azaspiro[bicyclo[3.2.1]octane-8,3′-pyrrolidin]-2′-one hydrochloride (25 mg, 69 μmol, 1.0 equiv.) and 6-chloro-1-(2,2-difluoroethyl)-1H-pyrazolo[3,4-b]pyrazine (15 mg, 69 μmol, 1.0 equiv.) as the starting materials to give 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.2.1]octane-8,3′-pyrrolidin]-2′-one (9 mg, 25%) as a white solid. ¹H NMR (400 MHz, Chloroform-d) δ 8.81 (d, J=2.5 Hz, 1H), 8.53 (dd, J=8.7, 2.5 Hz, 1H), 8.19 (s, 1H), 8.08 (s, 1H), 7.68 (d, J=8.7 Hz, 1H), 6.23 (tt, J=55.6, 4.5 Hz, 1H), 4.66 (td, J=13.3, 4.5 Hz, 2H), 4.07-3.97 (m, 2H), 3.97-3.85 (m, 4H), 2.54-2.45 (m, 2H), 2.14 (t, J=6.9 Hz, 2H), 2.05-1.93 (m, 2H), 1.82-1.71 (m, 2H). MS m/z: 508.25 [M+H]⁺.

### Example 800: 3-(1-(2,2-difluoroethyl)-1H-pyrazolo[3,4-b]pyrazin-6-yl)-1′-(5-(trifluoromethyl)pyridin-2-yl)-3-azaspiro[bicyclo[3.2.1]octane-8,3′-pyrrolidin]-2′-one

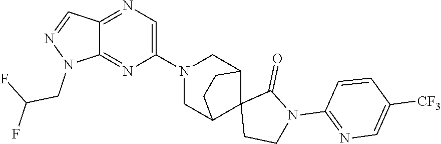 [SMILES: O=C1N(c2ccc(C(F)(F)F)cn2)CCC12C1CCC2CN(c2cnc3cnn(CC(F)F)c3n2)C1]

Step 1: tert-butyl 2′-oxo-1′-(5-(trifluoromethyl)pyridin-2-yl)-3-azaspiro[bicyclo[3.2.1]octane-8,3′-pyrrolidine]-3-carboxylate: Followed the general procedure E using tert-butyl 2′-oxo-3-azaspiro[bicyclo[3.2.1]octane-8,3′-pyrrolidine]-3-carboxylate (100 mg, 0.356 mmol, 1.0 equiv.) and 2-fluoro-5-(trifluoromethyl)pyridine (88 mg, 0.536 mmol, 1.5 equiv.) as the starting materials to give tert-butyl 2′-oxo-1′-(5-(trifluoromethyl)pyridin-3-yl)-3-azaspiro[bicyclo[3.2.1]octane-8,3′-pyrrolidine]-3-carboxylate (80 mg, 53%) as a colorless oil. MS m/z: 426 [M+H]⁺.

Step 2: 1′-(5-(trifluoromethyl)pyridin-2-yl)-3-azaspiro[bicyclo[3.2.1]octane-8,3′-pyrrolidin]-2′-one hydrochloride: Followed the general procedure B using tert-butyl 2′-oxo-1′-(5-(trifluoromethyl)pyridin-2-yl)-3-azaspiro[bicyclo[3.2.1]octane-8,3′-pyrrolidine]-3-carboxylate (80 mg) as the starting material to give the crude product 1′-(5-(trifluoromethyl)pyridin-2-yl)-3-azaspiro[bicyclo[3.2.1]octane-8,3′-pyrrolidin]-2′-one hydrochloride (65 mg). MS m/z: 326 [M+H]⁺.

Step 3: 3-(1-(2,2-difluoroethyl)-1H-pyrazolo[3,4-b]pyrazin-6-yl)-1′-(5-(trifluoromethyl)pyridin-2-yl)-3-azaspiro[bicyclo[3.2.1]octane-8,3′-pyrrolidin]-2′-one: Followed the general procedure I using 1′-(5-(trifluoromethyl)pyridin-2-yl)-3-azaspiro[bicyclo[3.2.1]octane-8,3′-pyrrolidin]-2′-one hydrochloride (65 mg, 180 μmol, 1.0 equiv.) and 5-bromo-2-(trifluoromethyl)pyridine (39 mg, 180 μmol, 1.0 equiv.) as the starting materials to give 3-(1-(2,2-difluoroethyl)-1H-pyrazolo[3,4-b]pyrazin-6-yl)-1′-(5-(trifluoromethyl)pyridin-2-yl)-3-azaspiro[bicyclo[3.2.1]octane-8,3′-pyrrolidin]-2′-one (25 mg, 27%) as a white solid. ¹H NMR (400 MHz, Chloroform-d) δ 8.65 (s, 1H), 8.55 (d, J=8.9 Hz, 1H), 8.23-8.14 (m, 2H), 7.89 (dd, J=8.9, 2.4 Hz, 1H), 6.23 (tt, J=55.5, 4.5 Hz, 1H), 4.69 (td, J=13.2, 4.4 Hz, 2H), 4.11 (t, J=7.0 Hz, 2H), 4.03-3.90 (m, 4H), 2.51 (s, 2H), 2.07 (t, J=7.1 Hz, 2H), 2.04-1.98 (m, 2H), 1.80-1.71 (m, 2H). MS m/z: 508.2 [M+H]⁺.

### Example 801: 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.1.1]heptane-6,3′-pyrrolidin]-5′-one

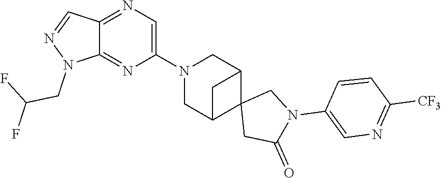 [SMILES: O=C1CC2(CN1c1ccc(C(F)(F)F)nc1)C1CC2CN(c2cnc3cnn(CC(F)F)c3n2)C1]

Step 1: benzyl 5′-oxo-1′-(6-(trifluoromethyl)pyridin-3-yl)-3-azaspiro[bicyclo[3.1.1]heptane-6,3′-pyrrolidine]-3-carboxylate: Followed the general procedure Y using benzyl 5′-oxo-3-azaspiro[bicyclo[3.1.1]heptane-6,3′-pyrrolidine]-3-carboxylate (150 mg, 499 μmol, 1.5 equiv.) and 5-bromo-2-(trifluoromethyl)pyridine (169 mg, 748 μmol, 1.5 equiv.) as the starting materials to give benzyl 5′-oxo-1′-(6-(trifluoromethyl)pyridin-3-yl)-3-azaspiro[bicyclo[3.1.1]heptane-6,3′-pyrrolidine]-3-carboxylate (50 mg, 22%) as a yellow oil. MS m/z: 446 [M+H]⁺.

Step 2: 1′-(6-(trifluoromethyl)pyridin-3-yl)-3-azaspiro[bicyclo[3.1.1]heptane-6,3′-pyrrolidin]-5′-one: Followed the general procedure U using benzyl 5′-oxo-1′-(6-(trifluoromethyl)pyridin-3-yl)-3-azaspiro[bicyclo[3.1.1]heptane-6,3′-pyrrolidine]-3-carboxylate (50 mg, 112 μmol) as the starting material to give 1′-(6-(trifluoromethyl)pyridin-3-yl)-3-azaspiro[bicyclo[3.1.1]heptane-6,3′-pyrrolidin]-5′-one (40 mg) as a yellow oil. MS m/z: 312 [M+H]⁺.

Step 3: 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.1.1]heptane-6,3′-pyrrolidin]-5′-one: Followed the general procedure I using 1′-(6-(trifluoromethyl)pyridin-3-yl)-3-azaspiro[bicyclo[3.1.1]heptane-6,3′-pyrrolidin]-5′-one (40 mg, 128 μmol, 1.0 equiv.) and 6-chloro-1-(2,2-difluoroethyl)-1H-pyrazolo[3,4-b]pyrazine (33.7 mg, 154 μmol, 1.2 equiv.) as the starting materials to give 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.1.1]heptane-6,3′-pyrrolidin]-5′-one (26.4 mg, 42%) as a white solid. ¹H NMR (400 MHz, DMSO-d₆) δ 9.13-9.05 (m, 1H), 8.43 (dd, J=8.7, 2.5 Hz, 1H), 8.27 (s, 1H), 8.18 (s, 1H), 7.96 (d, J=8.7 Hz, 1H), 6.49 (tt, J=55.0, 4.1 Hz, 1H), 4.73 (td, J=14.9, 4.0 Hz, 2H), 4.28 (s, 2H), 3.88 (s, 4H), 2.66-2.60 (m, 2H), 2.55 (s, 2H), 2.47 (s, 1H), 1.53 (d, J=10.0 Hz, 1H). MS m/z: 535.1 [M+H]⁺.

### Example 802: 3-(1-(2,2-difluoroethyl)-1H-pyrazolo[3,4-b]pyrazin-6-yl)-1′-(5-(trifluoromethyl)pyridin-2-yl)-3-azaspiro[bicyclo[3.1.1]heptane-6,3′-pyrrolidin]-5′-one

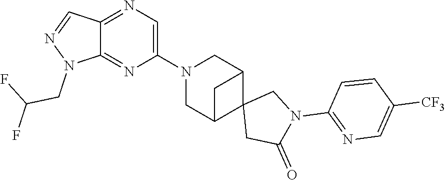 [SMILES: O=C1CC2(CN1c1ccc(C(F)(F)F)cn1)C1CC2CN(c2cnc3cnn(CC(F)F)c3n2)C1]

Step 1: benzyl 5′-oxo-1′-(5-(trifluoromethyl)pyridin-2-yl)-3-azaspiro[bicyclo[3.1.1]heptane-6,3′-pyrrolidine]-3-carboxylate: Followed the general procedure Y using benzyl 5′-oxo-3-azaspiro[bicyclo[3.1.1]heptane-6,3′-pyrrolidine]-3-carboxylate (150 mg, 0.5 mmol, 1.0 equiv.) and 2-bromo-5-(trifluoromethyl)pyridine (135 mg, 0.6 mmol, 1.2 equiv.) as the starting materials to give benzyl 5′-oxo-1′-(5-(trifluoromethyl)pyridin-2-yl)-3-azaspiro[bicyclo[3.1.1]heptane-6,3′-pyrrolidine]-3-carboxylate (70 mg, 32%) as a yellow solid. MS m/z: 456 [M+H]⁺.

Step 2: 1′-(5-(trifluoromethyl)pyridin-2-yl)-3-azaspiro[bicyclo[3.1.1]heptane-6,3′-pyrrolidin]-5′-one: Followed the general procedure U using benzyl 5′-oxo-1′-(5-(trifluoromethyl)pyridin-2-yl)-3-azaspiro[bicyclo[3.1.1]heptane-6,3′-pyrrolidine]-3-carboxylate (70 mg, 153 μmol) as the starting material to give 1′-(5-(trifluoromethyl)pyridin-2-yl)-3-azaspiro[bicyclo[3.1.1]heptane-6,3′-pyrrolidin]-5′-one (40 mg, 83%) as a yellow oil. MS m/z: 312 [M+H]⁺.

Step 3: 3-(1-(2,2-difluoroethyl)-1H-pyrazolo[3,4-b]pyrazin-6-yl)-1′-(5-(trifluoromethyl)pyridin-2-yl)-3-azaspiro[bicyclo[3.1.1]heptane-6,3′-pyrrolidin]-5′-one: Followed the general procedure I using 1′-(5-(trifluoromethyl)pyridin-2-yl)-3-azaspiro[bicyclo[3.1.1]heptane-6,3′-pyrrolidin]-5′-one (40 mg, 129 μmol, 1.0 equiv.) and 6-chloro-1-(2,2-difluoroethyl)-1H-pyrazolo[3,4-b]pyrazine (28 mg, 129 μmol, 1.0 equiv.) as the starting materials to give 3-(1-(2,2-difluoroethyl)-1H-pyrazolo[3,4-b]pyrazin-6-yl)-1′-(5-(trifluoromethyl)pyridin-2-yl)-3-azaspiro[bicyclo[3.1.1]heptane-6,3′-pyrrolidin]-5′-one (16.2 mg, 26%) as a yellow solid. ¹H NMR (400 MHz, DMSO-d₆) δ 8.75 (s, 1H), 8.44 (d, J=8.9 Hz, 1H), 8.26-8.09 (m, 3H), 6.41 (tt, J=54.9, 4.0 Hz, 1H), 4.66 (td, J=15.0, 3.9 Hz, 2H), 4.29 (s, 2H), 3.81 (br, 4H), 2.68-2.43 (m, 5H), 1.45 (d, J=10.0 Hz, 1H). MS m/z: 494.05 [M+H]⁺.

### Example 803: 1′-(1-(2,2-difluoroethyl)-1H-pyrazolo[3,4-b]pyrazin-6-yl)-8-(6-(trifluoromethyl)pyridin-3-yl)-8-azaspiro[bicyclo[3.2.1]octane-3,3′-pyrrolidin]-2′-one

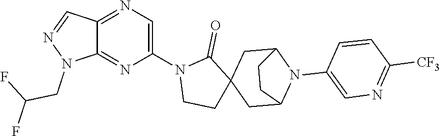 [SMILES: O=C1N(c2cnc3cnn(CC(F)F)c3n2)CCC12CC1CCC(C2)N1c1ccc(C(F)(F)F)nc1]

Step 1: 8-(tert-butyl) 3-methyl 3-(cyanomethyl)-8-azabicyclo[3.2.1]octane-3,8-dicarboxylate: Followed the general procedure AC using 8-(tert-butyl) 3-methyl 8-azabicyclo[3.2.1]octane-3,8-dicarboxylate (800 mg, 2.97 mmol, 1.0 equiv.) and 2-bromoacetonitrile (707 mg, 5.97 mmol, 2.0 equiv.) as the starting materials to give 8-(tert-butyl) 3-methyl 3-(cyanomethyl)-8-azabicyclo[3.2.1]octane-3,8-dicarboxylate (400 mg, 43%) as a colorless oil. MS m/z: 309 [M+H]⁺.

Step 2: tert-butyl 2′-oxo-8-azaspiro[bicyclo[3.2.1]octane-3,3′-pyrrolidine]-8-carboxylate: Followed the general procedure AB using 8-(tert-butyl) 3-methyl 3-(cyanomethyl)-8-azabicyclo[3.2.1]octane-3,8-dicarboxylate (400 mg, 1.29 mmol, 1.0 equiv.) as the starting material to give tert-butyl 2′-oxo-8-azaspiro[bicyclo[3.2.1]octane-3,3′-pyrrolidine]-8-carboxylate (250 mg, 68%) as a colorless oil. MS m/z: 281 [M+H]⁺.

Step 3: tert-butyl 1′-(1-(2,2-difluoroethyl)-1H-pyrazolo[3,4-b]pyrazin-6-yl)-2′-oxo-8-azaspiro[bicyclo[3.2.1]octane-3,3′-pyrrolidine]-8-carboxylate: Followed the general procedure Y using tert-butyl 2′-oxo-8-azaspiro[bicyclo[3.2.1]octane-3,3′-pyrrolidine]-8-carboxylate (300 mg, 1.07 mmol, 1.0 equiv.) and 6-chloro-1-(2,2-difluoroethyl)-1H-pyrazolo[3,4-b]pyrazine (233 mg, 1.07 mmol, 1.0 equiv.) as the starting materials to give tert-butyl 1′-(1-(2,2-difluoroethyl)-1H-pyrazolo[3,4-b]pyrazin-6-yl)-2′-oxo-8-azaspiro[bicyclo[3.2.1]octane-3,3′-pyrrolidine]-8-carboxylate (150 mg, 30%) as a colorless oil. MS m/z: 463 [M+H]⁺.

Step 4: 1′-(1-(2,2-difluoroethyl)-1H-pyrazolo[3,4-b]pyrazin-6-yl)-8-azaspiro[bicyclo[3.2.1]octane-3,3′-pyrrolidin]-2′-one hydrochloride: Followed the general procedure B using tert-butyl 1′-(1-(2,2-difluoroethyl)-1H-pyrazolo[3,4-b]pyrazin-6-yl)-2′-oxo-8-azaspiro[bicyclo[3.2.1]octane-3,3′-pyrrolidine]-8-carboxylate (150 mg) as the starting material to give the crude product 1′-(1-(2,2-difluoroethyl)-1H-pyrazolo[3,4-b]pyrazin-6-yl)-8-azaspiro[bicyclo[3.2.1]octane-3,3′-pyrrolidin]-2′-one hydrochloride (120 mg). MS m/z: 363 [M+H]⁺.

Step 5: 1′-(1-(2,2-difluoroethyl)-1H-pyrazolo[3,4-b]pyrazin-6-yl)-8-(6-(trifluoromethyl)pyridin-3-yl)-8-azaspiro[bicyclo[3.2.1]octane-3,3′-pyrrolidin]-2′-one: Followed the general procedure Y using 1′-(1-(2,2-difluoroethyl)-1H-pyrazolo[3,4-b]pyrazin-6-yl)-8-azaspiro[bicyclo[3.2.1]octane-3,3′-pyrrolidin]-2′-one hydrochloride (40 mg, 100 μmol, 1.0 equiv.) and 5-bromo-2-(trifluoromethyl)pyridine (25 mg, 110 μmol, 1.1 equiv.) as the starting materials to give 1′-(1-(2,2-difluoroethyl)-1H-pyrazolo[3,4-b]pyrazin-6-yl)-8-(6-(trifluoromethyl)pyridin-3-yl)-8-azaspiro[bicyclo[3.2.1]octane-3,3′-pyrrolidin]-2′-one (16 mg, 31%) as a white solid. ¹H NMR (400 MHz, DMSO-d₆) δ 9.67 (s, 1H), 8.48 (s, 1H), 8.34 (d, J=2.8 Hz, 1H), 7.63 (d, J=8.7 Hz, 1H), 7.36 (dd, J=8.7, 2.8 Hz, 1H), 6.49 (tt, J=54.6, 3.6 Hz, 1H), 4.85 (td, J=15.1, 3.7 Hz, 2H), 4.56-4.45 (m, 2H), 3.91 (t, J=6.5 Hz, 2H), 2.34-2.25 (m, 2H), 2.05-1.92 (m, 6H), 1.80 (t, J=6.5 Hz, 2H). MS m/z: 508.15 [M+H]⁺.

### Example 804: 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.2.1]octane-8,3′-pyrrolidin]-5′-one

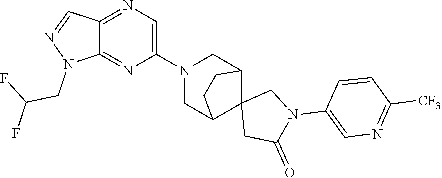 [SMILES: O=C1CC2(CN1c1ccc(C(F)(F)F)nc1)C1CCC2CN(c2cnc3cnn(CC(F)F)c3n2)C1]

Step 1: tert-butyl 5′-oxo-1′-(6-(trifluoromethyl)pyridin-3-yl)-3-azaspiro[bicyclo[3.2.1]octane-8,3′-pyrrolidine]-3-carboxylate: Followed the general procedure X using benzyl 5′-oxo-3-azaspiro[bicyclo[3.1.1]heptane-6,3′-pyrrolidine]-3-carboxylate (100 mg, 357 μmol, 1.0 equiv.) and 5-bromo-2-(trifluoromethyl)pyridine (121 mg, 535 μmol, 1.5 equiv.) as the starting materials, Xphos Pd G3 (60 mg, 71 μmol, 0.2 equiv.) as the catalyst to give tert-butyl 5′-oxo-1′-(6-(trifluoromethyl)pyridin-3-yl)-3-azaspiro[bicyclo[3.2.1]octane-8,3′-pyrrolidine]-3-carboxylate (80 mg, 53%) as a yellow solid. MS m/z: 426 [M+H]⁺.

Step 2: 1′-(6-(trifluoromethyl)pyridin-3-yl)-3-azaspiro[bicyclo[3.1.1]heptane-6,3′-pyrrolidin]-5′-one hydrochloride: Followed the general procedure B using tert-butyl 5′-oxo-1′-(6-(trifluoromethyl)pyridin-3-yl)-3-azaspiro[bicyclo[3.2.1]octane-8,3′-pyrrolidine]-3-carboxylate (80 mg) as the starting material to give the crude product 1′-(6-(trifluoromethyl)pyridin-3-yl)-3-azaspiro[bicyclo[3.1.1]heptane-6,3′-pyrrolidin]-5′-one hydrochloride (80 mg). MS m/z: 326 [M+H]⁺.

Step 3: 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.2.1]octane-8,3′-pyrrolidin]-5′-one: Followed the general procedure I using 1′-(6-(trifluoromethyl)pyridin-3-yl)-3-azaspiro[bicyclo[3.1.1]heptane-6,3′-pyrrolidin]-5′-one hydrochloride (74 mg, 229 μmol, 1.0 equiv.) and 6-chloro-1-(2,2-difluoroethyl)-1H-pyrazolo[3,4-b]pyrazine (50 mg, 229 μmol, 1.0 equiv.) as the starting materials to give 3-(1-(2,2-difluoroethyl)-1H-pyrazolo[3,4-b]pyrazin-6-yl)-1′-(6-(trifluoromethyl)pyridin-3-yl)-3-azaspiro[bicyclo[3.2.1]octane-8,3′-pyrrolidin]-5′-one (39.7 mg, 33%) as a white solid. ¹H NMR (400 MHz, DMSO-d₆) δ 8.82-8.77 (m, 1H), 8.54 (d, J=8.9 Hz, 1H), 8.39 (s, 1H), 8.23 (dd, J=9.0, 2.6 Hz, 1H), 8.15 (s, 1H), 6.45 (tt, J=55.0, 3.9 Hz, 1H), 4.70 (td, J=15.0, 3.9 Hz, 2H), 4.13 (d, J=12.8 Hz, 2H), 3.87 (s, 2H), 3.32-2.22 (m, 2H), 2.99 (s, 2H), 2.26 (s, 2H), 1.95-1.88 (m, 2H), 1.61 (d, J=8.2 Hz, 2H). MS m/z: 508.1 [M+H]⁺.

### Example 805: 3-(1-(2,2-difluoroethyl)-1H-pyrazolo[3,4-b]pyrazin-6-yl)-1′-(5-(trifluoromethyl)pyrazin-2-yl)-3-azaspiro[bicyclo[3.2.1]octane-8,3′-pyrrolidin]-5′-one

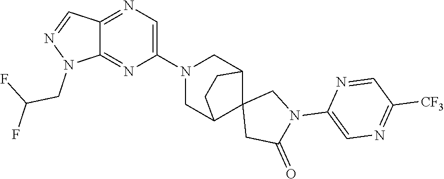 [SMILES: O=C1CC2(CN1c1cnc(C(F)(F)F)cn1)C1CCC2CN(c2cnc3cnn(CC(F)F)c3n2)C1]

Step 1: tert-butyl 5′-oxo-1′-(5-(trifluoromethyl)pyrazin-2-yl)-3-azaspiro[bicyclo[3.2.1]octane-8,3′-pyrrolidine]-3-carboxylate: Followed the general procedure X using tert-butyl 5′-oxo-3-azaspiro[bicyclo[3.2.1]octane-8,3′-pyrrolidine]-3-carboxylate (100 mg, 357 μmol, 1.0 equiv.) and 2-chloro-5-(trifluoromethyl)pyrazine (98 mg, 535 μmol, 1.5 equiv.) as the starting materials, Xphos Pd G3 (60 mg, 71 μmol, 0.2 equiv.) as the catalyst to give tert-butyl 5′-oxo-1′-(5-(trifluoromethyl)pyrazin-2-yl)-3-azaspiro[bicyclo[3.2.1]octane-8,3′-pyrrolidine]-3-carboxylate (80 mg, 53%) as a yellow oil. MS m/z: 427 [M+H]⁺.

Step 2: 1′-(5-(trifluoromethyl)pyrazin-2-yl)-3-azaspiro[bicyclo[3.2.1]octane-8,3′-pyrrolidin]-5′-one hydrochloride: Followed the general procedure B using tert-butyl 5′-oxo-1′-(5-(trifluoromethyl)pyrazin-2-yl)-3-azaspiro[bicyclo[3.2.1]octane-8,3′-pyrrolidine]-3-carboxylate (80 mg) as the starting material to give the crude product 1′-(5-(trifluoromethyl)pyrazin-2-yl)-3-azaspiro[bicyclo[3.2.1]octane-8,3′-pyrrolidin]-5′-one hydrochloride (80 mg). MS m/z: 327 [M+H]⁺.

Step 3: 3-(1-(2,2-difluoroethyl)-1H-pyrazolo[3,4-b]pyrazin-6-yl)-1′-(5-(trifluoromethyl)pyrazin-2-yl)-3-azaspiro[bicyclo[3.2.1]octane-8,3′-pyrrolidin]-5′-one: Followed the general procedure I using 1′-(5-(trifluoromethyl)pyrazin-2-yl)-3-azaspiro[bicyclo[3.2.1]octane-8,3′-pyrrolidin]-5′-one hydrochloride (90 mg, 274 μmol, 1.5 equiv.) and 6-chloro-1-(2,2-difluoroethyl)-1H-pyrazolo[3,4-b]pyrazine (40 mg, 183 μmol, 1.0 equiv.) as the starting materials to give 3-(1-(2,2-difluoroethyl)-1H-pyrazolo[3,4-b]pyrazin-6-yl)-1′-(5-(trifluoromethyl)pyrazin-2-yl)-3-azaspiro[bicyclo[3.2.1]octane-8,3′-pyrrolidin]-5′-one (44.2 mg, 48%) as a white solid. ¹H NMR (400 MHz, DMSO-d₆) δ 9.69 (s, 1H), 8.99 (s, 1H), 8.39 (s, 1H), 8.16 (s, 1H), 6.45 (tt, J=54.9, 3.7, 1H), 4.70 (td, J=14.9, 3.8 Hz, 2H), 4.14 (d, J=12.4 Hz, 2H), 3.83 (s, 2H), 3.32-3.26 (m, 2H), 3.03 (s, 2H), 2.29 (s, 2H), 1.95-1.88 (m, 2H), 1.64-1.58 (m, 2H). MS m/z: 509.10 [M+H]⁺.

### Example 806: 3-(1-(2,2-difluoroethyl)-1H-pyrazolo[3,4-b]pyrazin-6-yl)-1′-(6-(trifluoromethyl)pyrazin-2-yl)-3-azaspiro[bicyclo[3.2.1]octane-8,3′-pyrrolidin]-5′-one

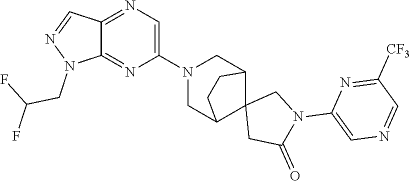 [SMILES: O=C1CC2(CN1c1cncc(C(F)(F)F)n1)C1CCC2CN(c2cnc3cnn(CC(F)F)c3n2)C1]

Step 1: tert-butyl 5′-oxo-1′-(6-(trifluoromethyl)pyrazin-2-yl)-3-azaspiro[bicyclo[3.2.1]octane-8,3′-pyrrolidine]-3-carboxylate: Followed the general procedure X using tert-butyl 5′-oxo-3-azaspiro[bicyclo[3.2.1]octane-8,3′-pyrrolidine]-3-carboxylate (100 mg, 357 μmol, 1.0 equiv.) and 2-chloro-6-(trifluoromethyl)pyrazine (98 mg, 535 μmol, 1.5 equiv.) as the starting materials, Xphos Pd G3 (60 mg, 71 μmol, 0.2 equiv.) as the catalyst to give tert-butyl 5′-oxo-1′-(6-(trifluoromethyl)pyrazin-2-yl)-3-azaspiro[bicyclo[3.2.1]octane-8,3′-pyrrolidine]-3-carboxylate (70 mg, 46%) as a yellow oil. MS m/z: 427 [M+H]⁺.

Step 2: 1′-(6-(trifluoromethyl)pyrazin-2-yl)-3-azaspiro[bicyclo[3.2.1]octane-8,3′-pyrrolidin]-5′-one hydrochloride: Followed the general procedure B using tert-butyl 5′-oxo-1′-(6-(trifluoromethyl)pyrazin-2-yl)-3-azaspiro[bicyclo[3.2.1]octane-8,3′-pyrrolidine]-3-carboxylate (70 mg) as the starting material to give the crude product 1′-(6-(trifluoromethyl)pyrazin-2-yl)-3-azaspiro[bicyclo[3.2.1]octane-8,3′-pyrrolidin]-5′-one hydrochloride (70 mg). MS m/z: 327 [M+H]⁺.

Step 3: 3-(1-(2,2-difluoroethyl)-1H-pyrazolo[3,4-b]pyrazin-6-yl)-1′-(6-(trifluoromethyl)pyrazin-2-yl)-3-azaspiro[bicyclo[3.2.1]octane-8,3′-pyrrolidin]-5′-one: Followed the general procedure I using 1′-(6-(trifluoromethyl)pyrazin-2-yl)-3-azaspiro[bicyclo[3.2.1]octane-8,3′-pyrrolidin]-5′-one hydrochloride (90 mg, 274 μmol, 1.5 equiv.) and 6-chloro-1-(2,2-difluoroethyl)-1H-pyrazolo[3,4-b]pyrazine (40 mg, 183 μmol, 1.0 equiv.) as the starting materials to give 3-(1-(2,2-difluoroethyl)-1H-pyrazolo[3,4-b]pyrazin-6-yl)-1′-(6-(trifluoromethyl)pyrazin-2-yl)-3-azaspiro[bicyclo[3.2.1]octane-8,3′-pyrrolidin]-5′-one (41.6 mg, 47%) as a white solid. ¹H NMR (400 MHz, DMSO-d₆) δ 9.82 (s, 1H), 8.90 (s, 1H), 8.39 (s, 1H), 8.16 (s, 1H), 6.46 (tt, J=54.9, 3.8 Hz, 1H), 4.70 (td, J=15.0, 3.9 Hz, 2H), 4.15 (d, 2H), 3.78 (s, 2H), 3.34-3.24 (m, 2H), 3.01 (s, 2H), 2.31 (s, 2H), 1.95-1.88 (m, 2H), 1.66-1.56 (m, 2H). MS m/z : 509.15 [M+H]⁺.

### Example 807: 3-(1-(2,2-difluoroethyl)-1H-pyrazolo[3,4-b]pyrazin-6-yl)-9-(6-(trifluoromethyl)pyridin-3-yl)-3,9-diazaspiro[5.5]undecane

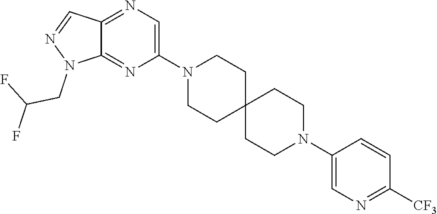 [SMILES: FC(F)Cn1ncc2ncc(N3CCC4(CCN(c5ccc(C(F)(F)F)nc5)CC4)CC3)nc21]

Step 1: tert-butyl 9-(6-(trifluoromethyl)pyridin-3-yl)-3,9-diazaspiro[5.5]undecane-3-carboxylate: Followed the general procedure Y using tert-butyl 3,9-diazaspiro[5.5]undecane-3-carboxylate (100 mg, 0.39 mmol, 1.0 equiv.) and 5-bromo-2-(trifluoromethyl)pyridine (106 mg, 0.47 mmol, 1.2 equiv.) as the starting materials EPhos Pd G4 (36 mg, 0.04 mmol, 0.1 equiv.) to give tert-butyl 9-(6-(trifluoromethyl)pyridin-3-yl)-3,9-diazaspiro[5.5]undecane-3-carboxylate (100 mg, 64%) as a colorless oil. MS m/z: 400 [M+H]⁺.

Step 2: 3-(6-(trifluoromethyl)pyridin-3-yl)-3,9-diazaspiro[5.5]undecane hydrochloride: Followed the general procedure B using tert-butyl 9-(6-(trifluoromethyl)pyridin-3-yl)-3,9-diazaspiro[5.5]undecane-3-carboxylate (100 mg) as the starting material to give the crude product 3-(6-(trifluoromethyl)pyridin-3-yl)-3,9-diazaspiro[5.5]undecane hydrochloride (100 mg). MS m/z: 300 [M+H]⁺.

Step 3: 3-(1-(2,2-difluoroethyl)-1H-pyrazolo[3,4-b]pyrazin-6-yl)-9-(6-(trifluoromethyl)pyridin-3-yl)-3,9-diazaspiro[5.5]undecane: Followed the general procedure I using 3-(6-(trifluoromethyl)pyridin-3-yl)-3,9-diazaspiro[5.5]undecane hydrochloride (70 mg, 0.234 mmol, 1.0 equiv.) and 6-chloro-1-(2,2-difluoroethyl)-1H-pyrazolo[3,4-b]pyrazine (56 mg, 0.257 mmol, 1.1 equiv.) as the starting materials to give 3-(1-(2,2-difluoroethyl)-1H-pyrazolo[3,4-b]pyrazin-6-yl)-9-(6-(trifluoromethyl)pyridin-3-yl)-3,9-diazaspiro[5.5]undecane (9 mg, 30%) as a white solid. ¹H NMR (400 MHz, DMSO-d₆) δ 8.48-8.40 (m, 2H), 8.13 (s, 1H), 7.62 (d, J=8.8 Hz, 1H), 7.44 (dd, J=8.8, 2.8 Hz, 1H), 6.61-6.25 (m, 1H), 4.69 (td, J=15.0, 3.8 Hz, 2H), 3.78 (t, J=5.8 Hz, 4H), 3.42 (d, J=11.6 Hz, 4H), 1.63 (dt, J=12.2, 5.4 Hz, 8H). MS m/z: 482.15 [M+H]⁺

### Example 808: 3-(1-(2,2-difluoroethyl)-1H-pyrazolo[3,4-b]pyrazin-6-yl)-9-(5-(trifluoromethyl)pyridin-3-yl)-3,9-diazaspiro[5.5]undecane

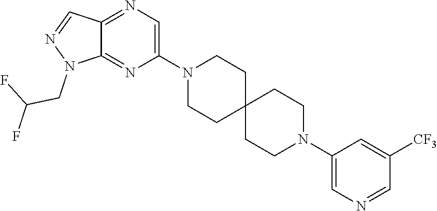 [SMILES: FC(F)Cn1ncc2ncc(N3CCC4(CCN(c5cncc(C(F)(F)F)c5)CC4)CC3)nc21]

Step 1: tert-butyl 9-(5-(trifluoromethyl)pyridin-3-yl)-3,9-diazaspiro[5.5]undecane-3-carboxylate: Followed the general procedure Y using tert-butyl 3,9-diazaspiro[5.5]undecane-3-carboxylate (120 mg, 0.472 mmol, 1.0 equiv.) and 3-bromo-5-(trifluoromethyl)pyridine (106 mg, 0.472 mmol, 1.0 equiv.) as the starting materials, XPhos (44.9 mg, 0.094 mmol, 0.2 equiv.), XPhos Pd G3 (39.9 mg, 0.047 mmol, 0.1 equiv.) as the catalysts to give tert-butyl 9-(5-(trifluoromethyl)pyridin-3-yl)-3,9-diazaspiro[5.5]undecane-3-carboxylate (160 mg, 84%) as a black solid. MS m/z: 400 [M+H]⁺.

Step 2: 3-(5-(trifluoromethyl)pyridin-3-yl)-3,9-diazaspiro[5.5]undecane hydrochloride: Followed the general procedure B using tert-butyl 9-(5-(trifluoromethyl)pyridin-3-yl)-3,9-diazaspiro[5.5]undecane-3-carboxylate (160 mg) as the starting material to give the crude product 3-(5-(trifluoromethyl)pyridin-3-yl)-3,9-diazaspiro[5.5]undecane hydrochloride (130 mg). MS m/z: 300 [M+H]⁺.

Step 3: 3-(1-(2,2-difluoroethyl)-1H-pyrazolo[3,4-b]pyrazin-6-yl)-9-(5-(trifluoromethyl)pyridin-3-yl)-3,9-diazaspiro[5.5]undecane: Followed the general procedure I using 3-(5-(trifluoromethyl)pyridin-3-yl)-3,9-diazaspiro[5.5]undecane hydrochloride (130 mg, 0.434 mmol, 1.0 equiv.) and 6-chloro-1-(2,2-difluoroethyl)-1H-pyrazolo[3,4-b]pyrazine (94.9 mg, 0.434 mmol, 1.0 equiv.) as the starting materials to give 3-(1-(2,2-difluoroethyl)-1H-pyrazolo[3,4-b]pyrazin-6-yl)-9-(5-(trifluoromethyl)pyridin-3-yl)-3,9-diazaspiro[5.5]undecane (77.2 mg, 18%) as a white solid. ¹H NMR (400 MHz, DMSO-d₆) δ 8.60 (d, J=2.8 Hz, 1H), 8.47 (s, 1H), 8.27-8.22 (m, 1H), 8.12 (s, 1H), 7.55 (t, J=2.4 Hz, 1H), 6.65-6.00 (m, 1H), 4.69 (td, J=15.0, 3.8 Hz, 2H), 3.82-3.74 (m, 4H), 3.41-3.34 (m, 4H), 1.68-1.57 (m, 8H). MS m/z: 482.15 [M+H]⁺.

### Example 809: 3-(1-(2,2-difluoroethyl)-1H-pyrazolo[3,4-b]pyrazin-6-yl)-9-(6-(trifluoromethyl)pyridin-2-yl)-3,9-diazaspiro[5.5]undecane

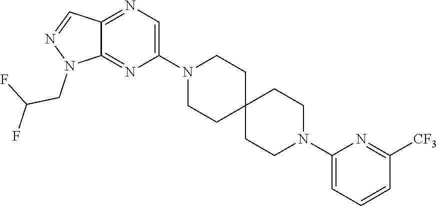 [SMILES: FC(F)Cn1ncc2ncc(N3CCC4(CCN(c5cccc(C(F)(F)F)n5)CC4)CC3)nc21]

Step 1: tert-butyl 9-(6-(trifluoromethyl)pyridin-2-yl)-3,9-diazaspiro[5.5]undecane-3-carboxylate: Followed the general procedure I using tert-butyl 3,9-diazaspiro[5.5]undecane-3-carboxylate (100 mg, 0.393 mmol, 1.0 equiv.) and 2,3-difluoro-5-(trifluoromethyl)pyridine (65 mg, 0.393 mmol, 1.0 equiv.) as the starting materials to give tert-butyl 9-(6-(trifluoromethyl)pyridin-2-yl)-3,9-diazaspiro[5.5]undecane-3-carboxylate (100 mg, 64%) as a white solid. MS m/z: 400 [M+H]⁺.

Step 2: 3-(6-(trifluoromethyl)pyridin-2-yl)-3,9-diazaspiro[5.5]undecane hydrochloride: Followed the general procedure B using tert-butyl 9-(6-(trifluoromethyl)pyridin-2-yl)-3,9-diazaspiro[5.5]undecane-3-carboxylate (100 mg) as the starting material to give the crude product 3-(6-(trifluoromethyl)pyridin-2-yl)-3,9-diazaspiro[5.5]undecane hydrochloride (75 mg). MS m/z: 300 [M+H]⁺.

Step 3: 3-(1-(2,2-difluoroethyl)-1H-pyrazolo[3,4-b]pyrazin-6-yl)-9-(6-(trifluoromethyl)pyridin-2-yl)-3,9-diazaspiro[5.5]undecane: Followed the general procedure I using 3-(6-(trifluoromethyl)748yridine-2-yl)-3,9-diazaspiro[5.5]undecane hydrochloride (75 mg, 0.25 mmol, 1 equiv.) and 6-chloro-1-(2,2-difluoroethyl)-1H-pyrazolo[3,4-b]pyrazine (55 mg, 0.25 mmol, 1 equiv.) as the starting materials to give 3-(1-(2,2-difluoroethyl)-1H-pyrazolo[3,4-b]pyrazin-6-yl)-9-(6-(trifluoromethyl)748yridine-2-yl)-3,9-diazaspiro[5.5]undecane (31.4 mg, 39%) as a white solid. ¹H NMR (400 MHz, DMSO-d₆) δ 8.47 (s, 1H), 8.12 (s, 1H), 7.77-7.66 (m, 1H), 7.13 (d, J=8.8 Hz, 1H), 6.99 (d, J=7.2 Hz, 1H), 6.44 (tt, J=54.9, 3.8 Hz, 1H), 4.69 (td, J=15.0, 3.8 Hz, 2H), 3.82-3.74 (m, 4H), 3.64-3.57 (m, 4H), 1.65-1.54 (m, 8H). MS m/z: 482.15 [M+H]⁺.

### Example 810: 3-(1-(2,2-difluoroethyl)-1H-pyrazolo[3,4-b]pyrazin-6-yl)-9-(5-(trifluoromethyl)pyridin-2-yl)-3,9-diazaspiro[5.5]undecane

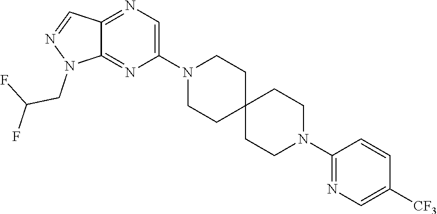 [SMILES: FC(F)Cn1ncc2ncc(N3CCC4(CCN(c5ccc(C(F)(F)F)cn5)CC4)CC3)nc21]

Step 1: tert-butyl 9-(5-(trifluoromethyl)pyridin-2-yl)-3,9-diazaspiro[5.5]undecane-3-carboxylate: Followed the general procedure I using tert-butyl 3,9-diazaspiro[5.5]undecane-3-carboxylate (461 mg, 1807 μmol, 1.0 equiv.) and 2-fluoro-5-(trifluoromethyl)pyridine (300 mg, 1807 μmol, 1.0 equiv.) as the starting materials to give tert-butyl 9-(5-(trifluoromethyl)pyridin-2-yl)-3,9-diazaspiro[5.5]undecane-3-carboxylate (240 mg, 33%) as a white solid. MS m/z: 400 [M+H]⁺.

Step 2: 3-(5-(trifluoromethyl)pyridin-2-yl)-3,9-diazaspiro[5.5]undecane: Followed the general procedure H using tert-butyl 9-(5-(trifluoromethyl)pyridin-2-yl)-3,9-diazaspiro[5.5]undecane-3-carboxylate (240 mg) as the starting material to give the crude product 3-(5-(trifluoromethyl)pyridin-2-yl)-3,9-diazaspiro[5.5]undecane (200 mg). MS m/z: 300 [M+H]⁺.

Step 3: 3-(1-(2,2-difluoroethyl)-1H-pyrazolo[3,4-b]pyrazin-6-yl)-9-(5-(trifluoromethyl)pyridin-2-yl)-3,9-diazaspiro[5.5]undecane: Followed the general procedure I using 3-(5-(trifluoromethyl)pyridin-2-yl)-3,9-diazaspiro[5.5]undecane (110 mg, 366 μmol, 1.0 equiv.) and 6-chloro-1-(2,2-difluoroethyl)-1H-pyrazolo[3,4-b]pyrazine (81 mg, 366 μmol, 1.0 equiv.) as the starting materials to give 3-(1-(2,2-difluoroethyl)-1H-pyrazolo[3,4-b]pyrazin-6-yl)-9-(5-(trifluoromethyl)pyridin-2-yl)-3,9-diazaspiro[5.5]undecane (60 mg, 33%) as a white solid. ¹H NMR (400 MHz, DMSO-d₆) δ 8.47 (s, 1H), 8.39 (d, J=2.6 Hz, 1H), 8.12 (s, 1H), 7.76 (dd, J=9.3, 2.6 Hz, 1H), 6.96 (d, J=9.1 Hz, 1H), 6.44 (tt, J=54.9, 3.8 Hz, 1H), 4.69 (td, J=15.0, 3.8 Hz, 2H), 3.81-3.74 (m, 4H), 3.68 (dd, J=6.9, 4.6 Hz, 4H), 1.65-1.53 (m, 8H). MS m/z: 482.25 [M+H]⁺.

### Example 811: 3-(1-(2,2-difluoroethyl)-1H-pyrazolo[3,4-b]pyrazin-6-yl)-9-(4-(trifluoromethyl)pyridin-2-yl)-3,9-diazaspiro[5.5]undecane

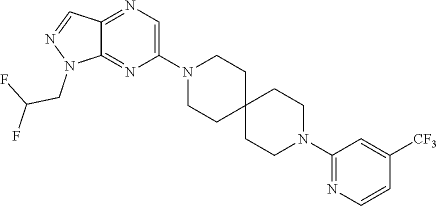 [SMILES: FC(F)Cn1ncc2ncc(N3CCC4(CCN(c5cc(C(F)(F)F)ccn5)CC4)CC3)nc21]

Step 1: tert-butyl 9-(4-(trifluoromethyl)pyridin-2-yl)-3,9-diazaspiro[5.5]undecane-3-carboxylate: Followed the general procedure I using tert-butyl 3,9-diazaspiro[5.5]undecane-3-carboxylate (150 mg, 0.590 mmol, 1 equiv) and 2-fluoro-4-(trifluoromethyl)pyridine (116 mg, 0.708 mmol, 1.2 equiv.) as the starting materials to give tert-butyl 9-(4-(trifluoromethyl)pyridin-2-yl)-3,9-diazaspiro[5.5]undecane-3-carboxylate (140 mg, 60%) as a white solid. MS m/z: 400 [M+H]⁺.

Step 2: 3-(4-(trifluoromethyl)pyridin-2-yl)-3,9-diazaspiro[5.5]undecane hydrochloride: Followed the general procedure B using tert-butyl 9-(4-(trifluoromethyl)pyridin-2-yl)-3,9-diazaspiro[5.5]undecane-3-carboxylate (140 mg, 0.350 mmol, 1 equiv) as the starting material to give the crude product 3-(4-(trifluoromethyl)pyridin-2-yl)-3,9-diazaspiro[5.5]undecane hydrochloride (100 mg). MS m/z: 300 [M+H]⁺.

Step 3: 3-(1-(2,2-difluoroethyl)-1H-pyrazolo[3,4-b]pyrazin-6-yl)-9-(4-(trifluoromethyl)pyridin-2-yl)-3,9-diazaspiro[5.5]undecane: Followed the general procedure I using 1-(2,3-difluorobenzyl)-3-(4-(trifluoromethyl)pyridin-2-yl)-1,3,8-triazaspiro[4.5]decane-2,4-dione hydrochloride (100 mg, 0.334 mmol, 1 equiv) and 6-chloro-1-(2,2-difluoroethyl)-1H-pyrazolo[3,4-b]pyrazine (87 mg, 0.401 mmol, 1.2 equiv) as the starting materials to give 3-(1-(2,2-difluoroethyl)-1H-pyrazolo[3,4-b]pyrazin-6-yl)-9-(4-(trifluoromethyl)pyridin-2-yl)-3,9-diazaspiro[5.5]undecane (30 mg, 21%) as a white solid. ¹H NMR (400 MHz, DMSO-d₆) δ 8.46 (s, 1H), 8.31 (d, J=5.1 Hz, 1H), 8.12 (s, 1H), 7.07 (s, 1H), 6.82 (dd, J=5.1, 1.4 Hz, 1H), 6.44 (tt, J=54.9, 3.8 Hz, 1H), 4.69 (td, J=15.0, 3.8 Hz, 2H), 3.86-3.72 (m, 4H), 3.70-3.57 (m, 4H), 1.72-1.48 (m, 8H). MS m/z: 482.15 [M+H]⁺.

### Example 812: 3-(1-(2,2-difluoroethyl)-1H-pyrazolo[3,4-b]pyrazin-6-yl)-9-(2-(trifluoromethyl)pyridin-4-yl)-3,9-diazaspiro[5.5]undecane

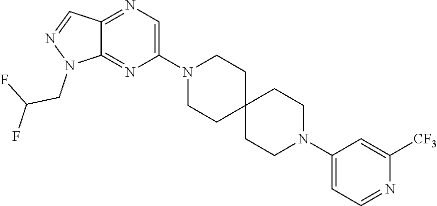 [SMILES: FC(F)Cn1ncc2ncc(N3CCC4(CCN(c5ccnc(C(F)(F)F)c5)CC4)CC3)nc21]

Step 1: tert-butyl 9-(2-(trifluoromethyl)pyridin-4-yl)-3,9-diazaspiro[5.5]undecane-3-carboxylate: Followed the general procedure I using tert-butyl 3,9-diazaspiro[5.5]undecane-3-carboxylate (100 mg, 0.393 mmol, 1.0 equiv.) and 4-chloro-2-(trifluoromethyl)pyridine (82 mg, 0.393 mmol, 1.0 equiv.) as the starting materials to give tert-butyl 9-(2-(trifluoromethyl)pyridin-4-yl)-3,9-diazaspiro[5.5]undecane-3-carboxylate (90 mg, 58%) as a white solid. MS m/z: 400 [M+H]⁺.

Step 2: 3-(2-(trifluoromethyl)pyridin-4-yl)-3,9-diazaspiro[5.5]undecane hydrochloride: Followed the general procedure B using tert-butyl 9-(2-(trifluoromethyl)pyridin-4-yl)-3,9-diazaspiro[5.5]undecane-3-carboxylate (90 mg) as the starting material to give the crude product 3-(2-(trifluoromethyl)pyridin-4-yl)-3,9-diazaspiro[5.5]undecane hydrochloride (60 mg). MS m/z: 300 [M+H]⁺.

Step 3: 3-(1-(2,2-difluoroethyl)-1H-pyrazolo[3,4-b]pyrazin-6-yl)-9-(2-(trifluoromethyl)pyridin-4-yl)-3,9-diazaspiro[5.5]undecane: Followed the general procedure I using 3-(2-(trifluoromethyl)pyridin-4-yl)-3,9-diazaspiro[5.5]undecane hydrochloride (60 mg, 0.2 mmol, 1.0 equiv.) and 6-chloro-1-(2,2-difluoroethyl)-1H-pyrazolo[3,4-b]pyrazine (45 mg, 0.2 mmol, 1.0 equiv.) as the starting materials to give 3-(1-(2,2-difluoroethyl)-1H-pyrazolo[3,4-b]pyrazin-6-yl)-9-(2-(trifluoromethyl)pyridin-4-yl)-3,9-diazaspiro[5.5]undecane (49.2 mg, 50%) as a white solid. ¹H NMR (400 MHz, DMSO-d₆) δ 8.47 (s, 1H), 8.25 (d, J=6.0 Hz, 1H), 8.12 (s, 1H), 7.20 (d, J=2.6 Hz, 1H), 7.04 (dd, J=6.1, 2.6 Hz, 1H), 6.44 (tt, J=54.9, 3.8 Hz, 1H), 4.69 (td, J=15.0, 3.8 Hz, 2H), 3.81-3.74 (m, 4H), 3.52-3.44 (m, 4H), 1.64-1.56 (m, 8H). MS m/z: 482.15 [M+H]⁺.

### Example 813: 3-(1-(2,2-difluoroethyl)-1H-pyrazolo[3,4-b]pyrazin-6-yl)-9-(6-(trifluoromethyl)pyrazin-2-yl)-3,9-diazaspiro[5.5]undecane

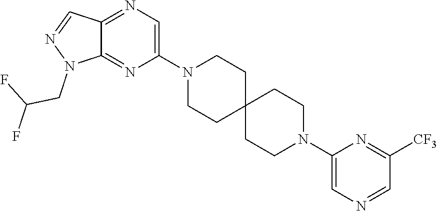 [SMILES: FC(F)Cn1ncc2ncc(N3CCC4(CCN(c5cncc(C(F)(F)F)n5)CC4)CC3)nc21]

Step 1: tert-butyl 9-(6-(trifluoromethyl)pyrazin-2-yl)-3,9-diazaspiro[5.5]undecane-3-carboxylate: Followed the general procedure I using tert-butyl 3,9-diazaspiro[5.5]undecane-3-carboxylate (100 mg, 392 μmol, 1.0 equiv.) and 2-chloro-6-(trifluoromethyl)pyrazine (71 mg, 392 μmol, 1.0 equiv.) as the starting materials to give tert-butyl 9-(6-(trifluoromethyl)pyrazin-2-yl)-3,9-diazaspiro[5.5]undecane-3-carboxylate (95 mg, 60%) as a white solid. MS m/z: 401 [M+H]⁺.

Step 2: 3-(6-(trifluoromethyl)pyrazin-2-yl)-3,9-diazaspiro[5.5]undecane: Followed the general procedure H using tert-butyl 9-(6-(trifluoromethyl)pyrazin-2-yl)-3,9-diazaspiro[5.5]undecane-3-carboxylate (95 mg) as the starting material to give the crude product 3-(6-(trifluoromethyl)pyrazin-2-yl)-3,9-diazaspiro[5.5]undecane (70 mg). MS m/z: 301 [M+H]⁺.

Step 3: 3-(1-(2,2-difluoroethyl)-1H-pyrazolo[3,4-b]pyrazin-6-yl)-9-(6-(trifluoromethyl)pyrazin-2-yl)-3,9-diazaspiro[5.5]undecane: Followed the general procedure I using 3-(6-(trifluoromethyl)pyrazin-2-yl)-3,9-diazaspiro[5.5]undecane (70 mg, 232 μmol, 1.0 equiv.) and 6-chloro-1-(2,2-difluoroethyl)-1H-pyrazolo[3,4-d]pyrimidine (51 mg, 300 μmol, 1.0 equiv.) as the starting materials to give 3-(1-(2,2-difluoroethyl)-1H-pyrazolo[3,4-b]pyrazin-6-yl)-9-(6-(trifluoromethyl)pyrazin-2-yl)-3,9-diazaspiro[5.5]undecane (65 mg, 58%) as a white solid. ¹H NMR (300 MHz, DMSO-d₆) δ 8.66 (s, 1H), 8.48 (s, 1H), 8.16 (d, J=16.5 Hz, 2H), 6.44 (tt, J=55.0, 3.8 Hz, 1H), 4.70 (td, J=15.0, 3.8 Hz, 2H), 3.79 (t, J=5.8 Hz, 4H), 3.70 (dd, J=7.0, 4.5 Hz, 4H), 1.62 (dt, J=8.2, 4.3 Hz, 8H). MS m/z: 483.15 [M+H]⁺.

### Example 814: 3-(1-(2,2-difluoroethyl)-1H-pyrazolo[3,4-b]pyrazin-6-yl)-9-(5-(trifluoromethyl)pyrazin-2-yl)-3,9-diazaspiro[5.5]undecane

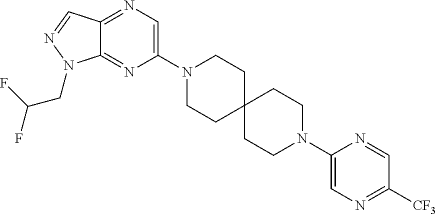 [SMILES: FC(F)Cn1ncc2ncc(N3CCC4(CCN(c5cnc(C(F)(F)F)cn5)CC4)CC3)nc21]

Step 1: tert-butyl 9-(5-(trifluoromethyl)pyrazin-2-yl)-3,9-diazaspiro[5.5]undecane-3-carboxylate: Followed the general procedure I using tert-butyl 3,9-diazaspiro[5.5]undecane-3-carboxylate (100 mg, 0.392 mmol, 1.0 equiv.) and 2-chloro-5-(trifluoromethyl)pyrazine (86.2 mg, 0.471 mmol, 1.2 equiv.) as the starting materials to give tert-butyl 9-(5-(trifluoromethyl)pyrazin-2-yl)-3,9-diazaspiro[5.5]undecane-3-carboxylate (100 mg, 57%) as a white solid. MS m/z: 401 [M+H]⁺.

Step 2: 3-(5-(trifluoromethyl)pyrazin-2-yl)-3,9-diazaspiro[5.5]undecane hydrochloride: Followed the general procedure B using tert-butyl 9-(5-(trifluoromethyl)pyrazin-2-yl)-3,9-diazaspiro[5.5]undecane-3-carboxylate (100 mg) as the starting material to give the crude product 3-(5-(trifluoromethyl)pyrazin-2-yl)-3,9-diazaspiro[5.5]undecane hydrochloride (65 mg). MS m/z: 301 [M+H]⁺.

Step 3: 3-(1-(2,2-difluoroethyl)-1H-pyrazolo[3,4-b]pyrazin-6-yl)-9-(5-(trifluoromethyl)pyrazin-2-yl)-3,9-diazaspiro[5.5]undecane: Followed the general procedure I using 3-(5-(trifluoromethyl)pyrazin-2-yl)-3,9-diazaspiro[5.5]undecane hydrochloride (65 mg, 0.216 mmol, 1.0 equiv.) and 6-chloro-1-(2,2-difluoroethyl)-1H-pyrazolo[3,4-b]pyrazine (52.1 mg, 0.238 mmol, 1.1 equiv.) as the starting materials to give 3-(1-(2,2-difluoroethyl)-1H-pyrazolo[3,4-b]pyrazin-6-yl)-9-(5-(trifluoromethyl)pyrazin-2-yl)-3,9-diazaspiro[5.5]undecane (61.3 mg, 58%) as a light yellow solid. ¹H NMR (400 MHz, DMSO-d₆) δ 8.50-8.42 (m, 3H), 8.12 (s, 1H), 6.44 (tt, J=54.9, 3.8 Hz, 1H), 4.76-4.63 (m, 2H), 3.82-3.72 (m, 8H), 1.66-1.57 (m, 8H). MS m/z: 483.15 [M+H]⁺.

### Example 815: 8-(1-(2,2-difluoroethyl)-1H-pyrazolo[3,4-b]pyrazin-6-yl)-3,3-dimethyl-2-(6-(trifluoromethyl)pyridin-3-yl)-2,8-diazaspiro[4.5]decan-1-one

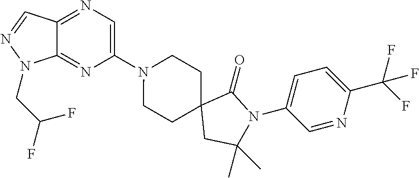 [SMILES: CC1(C)CC2(CCN(c3cnc4cnn(CC(F)F)c4n3)CC2)C(=O)N1c1ccc(C(F)(F)F)nc1]

Step 1: 8-benzyl-3,3-dimethyl-2-(6-(trifluoromethyl)pyridin-3-yl)-2,8-diazaspiro[4.5]decan-1-one: Followed the general procedure Y using 8-benzyl-3,3-dimethyl-2,8-diazaspiro[4.5]decan-1-one (130 mg, 0.48 mmol, 1.0 equiv.) and 5-bromo-2-(trifluoromethyl)pyridine (161 mg, 0.72 mmol, 1.5 equiv.) as the starting materials, 1612891-29-8 (20 mg, 0.024 mmol, 0.05 equiv.) as the catalyst to give 8-benzyl-3,3-dimethyl-2-(6-(trifluoromethyl)pyridin-3-yl)-2,8-diazaspiro[4.5]decan-1-one (120 mg, 60%) as a white solid. MS m/z: 418 [M+H]⁺.

Step 2: 3,3-dimethyl-2-(4-(trifluoromethyl)pyridin-2-yl)-2,8-diazaspiro[4.5]decan-1-one: Followed the general procedure U using 8-benzyl-3,3-dimethyl-2-(6-(trifluoromethyl)pyridin-3-yl)-2,8-diazaspiro[4.5]decan-1-one (120 mg, 0.29 mmol, 1.0 equiv.) as the starting material to give 3,3-dimethyl-2-(4-(trifluoromethyl)pyridin-2-yl)-2,8-diazaspiro[4.5]decan-1-one (80 mg, 84%) as a colorless oil. MS m/z: 328 [M+H]⁺.

Step 3: 8-(1-(2,2-difluoroethyl)-1H-pyrazolo[3,4-b]pyrazin-6-yl)-3,3-dimethyl-2-(6-(trifluoromethyl)pyridin-3-yl)-2,8-diazaspiro[4.5]decan-1-one: Followed the general procedure I using 3,3-dimethyl-2-(4-(trifluoromethyl)pyridin-2-yl)-2,8-diazaspiro[4.5]decan-1-one (80 mg, 0.24 mmol, 1.0 equiv.) and 6-chloro-1-(2,2-difluoroethyl)-1H-pyrazolo[3,4-b]pyrazine (58 mg, 0.27 mmol, 1.1 equiv.) as the starting materials to give 8-(1-(2,2-difluoroethyl)-1H-pyrazolo[3,4-b]pyrazin-6-yl)-3,3-dimethyl-2-(6-(trifluoromethyl)pyridin-3-yl)-2,8-diazaspiro[4.5]decan-1-one (66 mg, 53%) as a white solid. ¹H NMR (400 MHz, DMSO-d₆) δ 8.53 (s, 1H), 8.34 (d, J=2.3 Hz, 1H), 8.16 (s, 1H), 7.79 (d, J=8.4 Hz, 1H), 7.58 (dd, J=8.5, 2.4 Hz, 1H), 6.45 (tt, J=54.8, 3.8 Hz, 1H), 4.71 (td, J=15.0, 3.8 Hz, 2H), 4.57-4.49 (m, 2H), 3.31-3.26 (m, 2H), 2.27 (s, 2H), 2.05-1.96 (m, 2H), 1.94-1.87 (m, 2H), 1.45 (s, 6H). MS m/z: 510.1 [M+H]⁺.

### Example 816: 8-(1-(2,2-difluoroethyl)-1H-pyrazolo[3,4-b]pyrazin-6-yl)-1,1-dimethyl-2-(6-(trifluoromethyl)pyridin-3-yl)-2,8-diazaspiro[4.5]decan-3-one

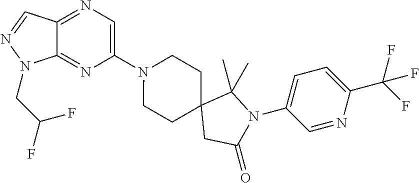 [SMILES: CC1(C)N(c2ccc(C(F)(F)F)nc2)C(=O)CC12CCN(c1cnc3cnn(CC(F)F)c3n1)CC2]

Step 1: 8-benzyl-1,1-dimethyl-2-(6-(trifluoromethyl)pyridin-3-yl)-2,8-diazaspiro[4.5]decan-3-one: Followed the general procedure Y using 8-benzyl-1,1-dimethyl-2,8-diazaspiro[4.5]decan-3-one (130 mg, 0.48 mmol, 1.0 equiv.) and 5-bromo-2-(trifluoromethyl)pyridine (161 mg, 0.72 mmol, 1.5 equiv.) as the starting materials, 1612891-29-8 (20 mg, 0.024 mmol, 0.05 equiv.) as the catalyst to give 8-benzyl-1,1-dimethyl-2-(6-(trifluoromethyl)pyridin-3-yl)-2,8-diazaspiro[4.5]decan-3-one (120 mg, 60%) as a white solid. MS m/z: 418 [M+H]⁺.

Step 2: 1,1-dimethyl-2-(6-(trifluoromethyl)pyridin-3-yl)-2,8-diazaspiro[4.5]decan-3-one: Followed the general procedure U using 8-benzyl-1,1-dimethyl-2-(6-(trifluoromethyl)pyridin-3-yl)-2,8-diazaspiro[4.5]decan-3-one (120 mg, 0.29 mmol, 1.0 equiv.) as the starting material to give 1,1-dimethyl-2-(6-(trifluoromethyl)pyridin-3-yl)-2,8-diazaspiro[4.5]decan-3-one (80 mg, 84%) as a colorless oil. MS m/z: 328 [M+H]⁺.

Step 3: 8-(1-(2,2-difluoroethyl)-1H-pyrazolo[3,4-b]pyrazin-6-yl)-1,1-dimethyl-2-(6-(trifluoromethyl)pyridin-3-yl)-2,8-diazaspiro[4.5]decan-3-one: Followed the general procedure I using 1,1-dimethyl-2-(6-(trifluoromethyl)pyridin-3-yl)-2,8-diazaspiro[4.5]decan-3-one (80 mg, 0.24 mmol, 1.0 equiv.) and 6-chloro-1-(2,2-difluoroethyl)-1H-pyrazolo[3,4-b]pyrazine (58 mg, 0.27 mmol, 1.1 equiv.) as the starting materials to give 8-(1-(2,2-difluoroethyl)-1H-pyrazolo[3,4-b]pyrazin-6-yl)-1,1-dimethyl-2-(6-(trifluoromethyl)pyridin-3-yl)-2,8-diazaspiro[4.5]decan-3-one (83 mg, 67%) as a white solid. ¹H NMR (400 MHz, DMSO-d₆) δ 8.54-8.29 (m, 2H), 8.13 (d, J=10.0 Hz, 1H), 7.86-7.77 (m, 1H), 7.63-7.55 (m, 1H), 6.62-6.28 (m, 1H), 4.77-4.44 (m, 4H), 3.20-2.83 (m, 4H), 1.68-1.54 (m, 4H), 1.32 (s, 2H), 1.26 (s, 4H). MS m/z: 510.2 [M+H]⁺.

### Example 817: 1-(3,5-difluorobenzyl)-8-(1-(2,2-difluoroethyl)-1H-pyrazolo[3,4-b]pyrazin-6-yl)-3-(2-(trifluoromethyl)pyridin-4-yl)-1,3,8-triazaspiro[4.5]decane-2,4-dione

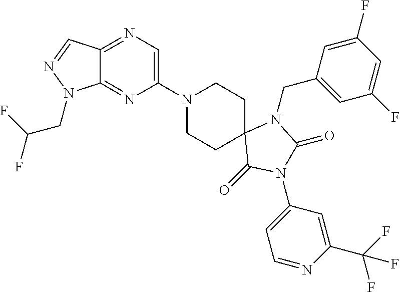 [SMILES: O=C1N(c2ccnc(C(F)(F)F)c2)C(=O)C2(CCN(c3cnc4cnn(CC(F)F)c4n3)CC2)N1Cc1cc(F)cc(F)c1]

Step 1: tert-butyl 2,4-dioxo-3-(2-(trifluoromethyl)pyridin-4-yl)-1,3,8-triazaspiro[4.5]decane-8-carboxylate: A mixture of 4-(((benzyloxy)carbonyl)amino)-1-(tert-butoxycarbonyl)piperidine-4-carboxylic acid (600 mg, 1.587 mmol, 1 equiv), 2-(trifluoromethyl)pyridin-4-amine (308 mg, 1.904 mmol, 1.2 equiv), TCFH (667 mg, 2.37 mmol, 1.5 equiv) and NMI (390 mg, 4.75 mmol, 3 equiv) in MeCN was stirred for 4 h at 80° C. under air atmosphere. The residue was purified by reversed-phase flash chromatography with the following conditions: column, C18 silica gel; mobile phase, MeCN in Water (0.1% FA), 0% to 100% gradient in 30 min; detector, UV 254 nm. This resulted in tert-butyl 2,4-dioxo-3-(2-(trifluoromethyl)pyridin-4-yl)-1,3,8-triazaspiro[4.5]decane-8-carboxylate (120 mg, 18%) as a white solid. MS m/z: 415 [M+H]⁺.

Step 2: tert-butyl 1-(3,5-difluorobenzyl)-2,4-dioxo-3-(2-(trifluoromethyl)pyridin-4-yl)-1,3,8-triazaspiro[4.5]decane-8-carboxylate: Followed the general procedure K using tert-butyl 2,4-dioxo-3-(2-(trifluoromethyl)pyridin-4-yl)-1,3,8-triazaspiro[4.5]decane-8-carboxylate (120 mg, 0.290 mmol, 1 equiv) and 1-(bromomethyl)-3,5-difluorobenzene (71 mg, 0.347 mmol, 1.2 equiv.) as the starting materials to give tert-butyl 1-(3,5-difluorobenzyl)-2,4-dioxo-3-(2-(trifluoromethyl)pyridin-4-yl)-1,3,8-triazaspiro[4.5]decane-8-carboxylate (80 mg, 51%) as a white solid. MS m/z: 541 [M+H]⁺.

Step 3: 1-(3,5-difluorobenzyl)-3-(2-(trifluoromethyl)pyridin-4-yl)-1,3,8-triazaspiro[4.5]decane-2,4-dione hydrochloride: Followed the general procedure B using tert-butyl 1-(3,5-difluorobenzyl)-2,4-dioxo-3-(2-(trifluoromethyl)pyridin-4-yl)-1,3,8-triazaspiro[4.5]decane-8-carboxylate (80 mg, 0.148 mmol, 1 equiv) as the starting material to give the crude product 1-(3,5-difluorobenzyl)-3-(2-(trifluoromethyl)pyridin-4-yl)-1,3,8-triazaspiro[4.5]decane-2,4-dione hydrochloride (60 mg). MS m/z: 441 [M+H]⁺.

Step 4: 1-(3,5-difluorobenzyl)-8-(1-(2,2-difluoroethyl)-1H-pyrazolo[3,4-b]pyrazin-6-yl)-3-(2-(trifluoromethyl)pyridin-4-yl)-1,3,8-triazaspiro[4.5]decane-2,4-dione: Followed the general procedure I using 1-(3,5-difluorobenzyl)-3-(2-(trifluoromethyl)pyridin-4-yl)-1,3,8-triazaspiro[4.5]decane-2,4-dione hydrochloride (60 mg, 0.136 mmol, 1 equiv) and 6-chloro-1-(2,2-difluoroethyl)-1H-pyrazolo[3,4-b]pyrazine (35 mg, 0.163 mmol, 1.2 equiv) as the starting materials to give 1-(3,5-difluorobenzyl)-8-(1-(2,2-difluoroethyl)-1H-pyrazolo[3,4-b]pyrazin-6-yl)-3-(2-(trifluoromethyl)pyridin-4-yl)-1,3,8-triazaspiro[4.5]decane-2,4-dione (50 mg, 59%) as a white solid. ¹H NMR (400 MHz, DMSO-d₆) δ 8.93 (d, J=5.4 Hz, 1H), 8.49 (s, 1H), 8.22 (d, J=1.9 Hz, 1H), 8.13 (s, 1H), 8.04 (dd, J=5.4, 1.9 Hz, 1H), 7.18-7.05 (m, 3H), 6.42 (tt, J=55.0, 3.8 Hz, 1H), 4.74-4.57 (m, 6H), 3.60 (t, J=12.8 Hz, 2H), 2.25-2.14 (m, 2H), 2.09-1.92 (m, 2H). MS m/z: 623.20 [M+H]⁺.

### Example 818: 1-(3,5-difluorobenzyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione

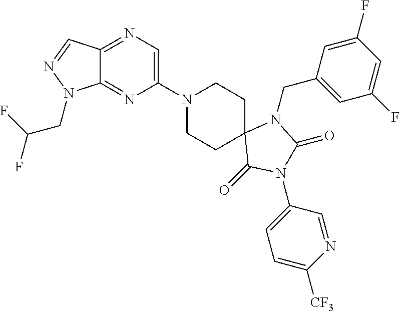 [SMILES: O=C1N(c2ccc(C(F)(F)F)nc2)C(=O)C2(CCN(c3cnc4cnn(CC(F)F)c4n3)CC2)N1Cc1cc(F)cc(F)c1]

Step 1: tert-butyl 4-(((benzyloxy)carbonyl)amino)-4-((6-(trifluoromethyl)pyridin-3-yl)carbamoyl)piperidine-1-carboxylate: Followed the general procedure G using 4-(((benzyloxy)carbonyl)amino)-1-(tert-butoxycarbonyl)piperidine-4-carboxylic acid (300 mg, 0.794 mmol, 1.0 equiv.) and 6-(trifluoromethyl)pyridin-3-amine (129 mg, 0.952 mmol, 1.2 equiv.) as the starting materials to give tert-butyl 4-(((benzyloxy)carbonyl)amino)-4-((6-(trifluoromethyl)pyridin-3-yl)carbamoyl)piperidine-1-carboxylate (170 mg, 41%) as a yellow oil. MS m/z: 523 [M+H]⁺.

Step 2: tert-butyl 2,4-dioxo-3-(6-(trifluoromethyl)pyridin-3-yl)-1,3,8-triazaspiro[4.5]decane-8-carboxylate: To a stirred solution of tert-butyl 4-(((benzyloxy)carbonyl)amino)-4-((6-(trifluoromethyl)pyridin-3-yl)carbamoyl)piperidine-1-carboxylate (170 mg, 0.325 mmol, 1 equiv.) in DMF (2 mL) at room temperature under nitrogen atmosphere. The resulting mixture was stirred for additional 2 h at 100° C. The resulting mixture was extracted with EtOAc (3×30 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (5:1) to afford tert-butyl 2,4-dioxo-3-[6-(trifluoromethyl)pyridin-3-yl]-1,3,8-triazaspiro[4.5]decane-8-carboxylate (65 mg, 48%) as a yellow oil. MS m/z: 415 [M+H]⁺.

Step 3: tert-butyl 1-(3,5-difluorobenzyl)-2,4-dioxo-3-(6-(trifluoromethyl)pyridin-3-yl)-1,3,8-triazaspiro[4.5]decane-8-carboxylate: Followed the general procedure K using 4-(((benzyloxy)carbonyl)amino)-1-(tert-butoxycarbonyl)piperidine-4-carboxylic acid (65 mg, 0.157 mmol, 1.0 equiv.) and 1-(bromomethyl)-3,5-difluorobenzene (42.0 mg, 0.204 mmol, 1.3 equiv.) as the starting materials to give tert-butyl 1-(3,5-difluorobenzyl)-2,4-dioxo-3-(6-(trifluoromethyl)pyridin-3-yl)-1,3,8-triazaspiro[4.5]decane-8-carboxylate (50 mg, 59%) as a yellow oil. MS m/z: 541 [M+H]⁺.

Step 4: 1-(3,5-difluorobenzyl)-3-(6-(trifluoromethyl)pyridin-3-yl)-1,3,8-triazaspiro[4.5]decane-2,4-dione hydrochloride: Followed the general procedure B using tert-butyl 3-methyl-2,8-diazaspiro[4.5]dec-2-ene-8-carboxylate (50 mg) as the starting material to give 1-(3,5-difluorobenzyl)-3-(6-(trifluoromethyl)pyridin-3-yl)-1,3,8-triazaspiro[4.5]decane-2,4-dione hydrochloride (40 mg) as a yellow solid. MS m/z: 441 [M+H]⁺.

Step 5: 1-(3,5-difluorobenzyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione: Followed the general procedure I using 1-(3,5-difluorobenzyl)-3-(6-(trifluoromethyl)pyridin-3-yl)-1,3,8-triazaspiro[4.5]decane-2,4-dione hydrochloride (40 mg, 0.09 mmol, 1.0 equiv.) and 6-chloro-1-(2,2-difluoroethyl)-1H-pyrazolo[3,4-b]pyrazine (78.6 mg, 0.1 mmol, 1.1 equiv.) as the starting materials to give 1-(3,5-difluorobenzyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione (18.3 mg, 32%) as a white solid. ¹H NMR (400 MHz, DMSO-d₆) δ 8.98 (d, J=2.3 Hz, 1H), 8.49 (s, 1H), 8.32 (dd, J=8.5, 2.3 Hz, 1H), 8.17-8.12 (m, 2H), 7.17-7.07 (m, 3H), 6.42 (tt, J=55.0, 3.8 Hz, 1H), 4.75-4.58 (m, 6H), 3.61 (t, J=12.8 Hz, 2H), 2.21 (d, J=13.1 Hz, 2H), 2.09-1.97 (m, 2H). MS m/z: 623.05 [M+H]⁺.

### Example 819: 1-(3,5-difluorobenzyl)-8-(1-(2,2-difluoroethyl)-1H-pyrazolo[3,4-b]pyrazin-6-yl)-3-(2-(trifluoromethyl)pyrimidin-5-yl)-1,3,8-triazaspiro[4.5]decane-2,4-dione

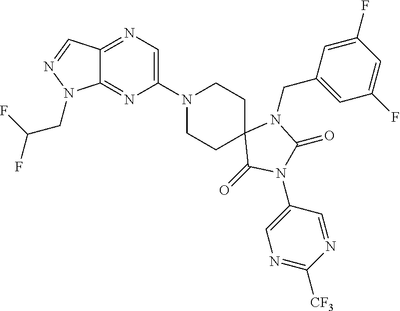 [SMILES: O=C1N(c2cnc(C(F)(F)F)nc2)C(=O)C2(CCN(c3cnc4cnn(CC(F)F)c4n3)CC2)N1Cc1cc(F)cc(F)c1]

Step 1: tert-butyl 4-(((benzyloxy)carbonyl)amino)-4-((2-(trifluoromethyl)pyrimidin-5-yl)carbamoyl)piperidine-1-carboxylate: Followed the general procedure G using 4-(((benzyloxy)carbonyl)amino)-1-(tert-butoxycarbonyl)piperidine-4-carboxylic acid (300 mg, 0.794 mmol, 1.0 equiv.) and 2-(trifluoromethyl)pyrimidin-5-amine (155 mg, 0.952 mmol, 1.2 equiv.) as the starting materials to give tert-butyl 4-(((benzyloxy)carbonyl)amino)-4-((2-(trifluoromethyl)pyrimidin-5-yl)carbamoyl)piperidine-1-carboxylate (85 mg, 20%) as a yellow oil. MS m/z: 524 [M+H]⁺.

Step 2: tert-butyl 2,4-dioxo-3-(6-(trifluoromethyl)pyridin-3-yl)-1,3,8-triazaspiro[4.5]decane-8-carboxylate: To a stirred solution of tert-butyl 4-(((benzyloxy)carbonyl)amino)-4-((2-(trifluoromethyl)pyrimidin-5-yl)carbamoyl)piperidine-1-carboxylate (85 mg, 0.162 mmol, 1 equiv.) in DMF (2 mL) at room temperature under nitrogen atmosphere. The resulting mixture was stirred for additional 2 h at 100° C. The resulting mixture was extracted with EtOAc (3×30 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (5:1) to afford tert-butyl 2,4-dioxo-3-[2-(trifluoromethyl)pyrimidin-5-yl]-1,3,8-triazaspiro[4.5]decane-8-carboxylate (55 mg, 82%) as a yellow oil. MS m/z: 416 [M+H]⁺.

Step 3: tert-butyl 1-(3,5-difluorobenzyl)-2,4-dioxo-3-(2-(trifluoromethyl)pyrimidin-5-yl)-1,3,8-triazaspiro[4.5]decane-8-carboxylate: Followed the general procedure K using 4-(((benzyloxy)carbonyl)amino)-1-(tert-butoxycarbonyl)piperidine-4-carboxylic acid (55 mg, 0.133 mmol, 1.0 equiv.) and 1-(bromomethyl)-3,5-difluorobenzene (35.5 mg, 0.172 mmol, 1.3 equiv.) as the starting materials to give tert-butyl 1-(3,5-difluorobenzyl)-2,4-dioxo-3-(2-(trifluoromethyl)pyrimidin-5-yl)-1,3,8-triazaspiro[4.5]decane-8-carboxylate (40 mg, 56%) as a yellow oil. MS m/z: 542 [M+H]⁺.

Step 4: 1-(3,5-difluorobenzyl)-3-(2-(trifluoromethyl)pyrimidin-5-yl)-1,3,8-triazaspiro[4.5]decane-2,4-dione hydrochloride: Followed the general procedure B using tert-butyl 3-methyl-2,8-diazaspiro[4.5]dec-2-ene-8-carboxylate (40 mg) as the starting material to give 1-(3,5-difluorobenzyl)-3-(2-(trifluoromethyl)pyrimidin-5-yl)-1,3,8-triazaspiro[4.5]decane-2,4-dione hydrochloride (30 mg) as a yellow solid. MS m/z: 442 [M+H]⁺.

Step 5: 1-(3,5-difluorobenzyl)-8-(1-(2,2-difluoroethyl)-1H-pyrazolo[3,4-b]pyrazin-6-yl)-3-(2-(trifluoromethyl)pyrimidin-5-yl)-1,3,8-triazaspiro[4.5]decane-2,4-dione: Followed the general procedure I using 1-(3,5-difluorobenzyl)-3-(2-(trifluoromethyl)pyrimidin-5-yl)-1,3,8-triazaspiro[4.5]decane-2,4-dione hydrochloride (30 mg, 0.07 mmol, 1.0 equiv.) and 6-chloro-1-(2,2-difluoroethyl)-1H-pyrazolo[3,4-b]pyrazine (78.6 mg, 0.08 mmol, 1.2 equiv.) as the starting materials to give 1-(3,5-difluorobenzyl)-8-(1-(2,2-difluoroethyl)-1H-pyrazolo[3,4-b]pyrazin-6-yl)-3-(2-(trifluoromethyl)pyrimidin-5-yl)-1,3,8-triazaspiro[4.5]decane-2,4-dione (15.5 mg, 36%) as a white solid. MS m/z: 400 [M+H]⁺. ¹H NMR (300 MHz, DMSO-d₆) δ 9.33 (s, 2H), 8.51 (s, 1H), 8.15 (s, 1H), 7.18-7.09 (m, 3H), 6.43 (tt, J=55.0, 3.8 Hz, 1H), 4.77-4.60 (m, 6H), 3.60 (t, J=12.7 Hz, 2H), 2.21 (d, J=13.0 Hz, 2H), 2.11-1.98 (m, 2H). MS m/z: 624.05 [M+H]⁺.

### Example 820: 1-(3,5-difluorobenzyl)-8-(1-(2,2-difluoroethyl)-1H-pyrazolo[3,4-b]pyrazin-6-yl)-3-(6-(trifluoromethyl)pyrazin-2-yl)-1,3,8-triazaspiro[4.5]decane-2,4-dione

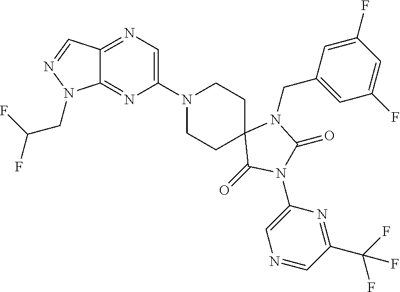 [SMILES: O=C1N(c2cncc(C(F)(F)F)n2)C(=O)C2(CCN(c3cnc4cnn(CC(F)F)c4n3)CC2)N1Cc1cc(F)cc(F)c1]

Step 1: tert-butyl 2,4-dioxo-3-(6-(trifluoromethyl)pyrazin-2-yl)-1,3,8-triazaspiro[4.5]decane-8-carboxylate: Followed the general procedure I using tert-butyl 2,4-dioxo-1,3,8-triazaspiro[4.5]decane-8-carboxylate (200 mg, 0.743 mmol, 1 equiv.) and 2-chloro-6-(trifluoromethyl)pyrazine (163 mg, 0.891 mmol, 1.2 equiv.) as the starting materials to give tert-butyl 2,4-dioxo-3-(6-(trifluoromethyl)pyrazin-2-yl)-1,3,8-triazaspiro[4.5]decane-8-carboxylate (70 mg, 23%) as a white solid. MS m/z: 416 [M+H]⁺.

Step 2: tert-butyl 1-(3,5-difluorobenzyl)-2,4-dioxo-3-(6-(trifluoromethyl)pyrazin-2-yl)-1,3,8-triazaspiro[4.5]decane-8-carboxylate: Followed the general procedure K using tert-butyl 2,4-dioxo-3-(6-(trifluoromethyl)pyrazin-2-yl)-1,3,8-triazaspiro[4.5]decane-8-carboxylate (70 mg, 0.168 mmol, 1.0 equiv.) and 1-(bromomethyl)-3,5-difluorobenzene (41.7 mg, 0.202 mmol, 1.2 equiv.) as the starting materials to give tert-butyl 1-(3,5-difluorobenzyl)-2,4-dioxo-3-(6-(trifluoromethyl)pyrazin-2-yl)-1,3,8-triazaspiro[4.5]decane-8-carboxylate (60 mg, 69%) as a white solid. MS m/z: 542 [M+H]⁺.

Step 3: 1-(3,5-difluorobenzyl)-3-(6-(trifluoromethyl)pyrazin-2-yl)-1,3,8-triazaspiro[4.5]decane-2,4-dione hydrochloride: Followed the general procedure B using tert-butyl 1-(3,5-difluorobenzyl)-2,4-dioxo-3-(6-(trifluoromethyl)pyrazin-2-yl)-1,3,8-triazaspiro[4.5]decane-8-carboxylate (60 mg) as the starting material to give the crude product 1-(3,5-difluorobenzyl)-3-(6-(trifluoromethyl)pyrazin-2-yl)-1,3,8-triazaspiro[4.5]decane-2,4-dione hydrochloride (45 mg). MS m/z: 442 [M+H]⁺.

Step 4: 1-(3,5-difluorobenzyl)-8-(1-(2,2-difluoroethyl)-1-pyrazolo[3,4-b]pyrazin-6-yl)-3-(6-(trifluoromethyl)pyrazin-2-yl)-1,3,8-triazaspiro[4.5]decane-2,4-dione: Followed the general procedure I using 1-(3,5-difluorobenzyl)-3-(6-(trifluoromethyl)pyrazin-2-yl)-1,3,8-triazaspiro[4.5]decane-2,4-dione hydrochloride (45 mg, 0.102 mmol, 1.0 equiv.) and 6-chloro-1-(2,2-difluoroethyl)-1H-pyrazolo[3,4-b]pyrazine (22 mg, 0.102 mmol, 1.0 equiv.) as the starting materials to give 1-(3,5-difluorobenzyl)-8-(1-(2,2-difluoroethyl)-1H-pyrazolo[3,4-b]pyrazin-6-yl)-3-(6-(trifluoromethyl)pyrazin-2-yl)-1,3,8-triazaspiro[4.5]decane-2,4-dione (32 mg, 50%) as a white solid. ¹H NMR (300 MHz, DMSO-d₆) δ 9.31 (s, 1H), 9.26 (s, 1H), 8.50 (s, 1H), 8.15 (s, 1H), 7.15 (d, J=7.9 Hz, 3H), 6.70-6.12 (m, 1H), 4.79-4.59 (m, 6H), 3.60 (t, J=12.6 Hz, 2H), 2.23 (d, J=13.5 Hz, 2H), 2.13-1.99 (m, 2H). MS m/z: 624.2 [M+H]⁺.

### Example 821: 1-(3,5-difluorobenzyl)-8-(1-(2,2-difluoroethyl)-1H-pyrazolo[3,4-b]pyrazin-6-yl)-3-(5-(trifluoromethyl)pyrazin-2-yl)-1,3,8-triazaspiro[4.5]decane-2,4-dione

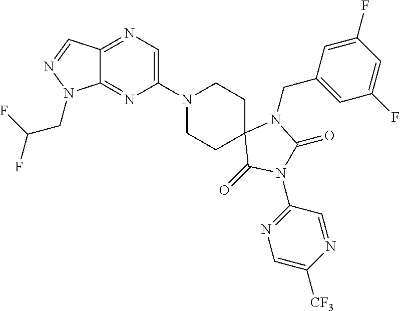 [SMILES: O=C1N(c2cnc(C(F)(F)F)cn2)C(=O)C2(CCN(c3cnc4cnn(CC(F)F)c4n3)CC2)N1Cc1cc(F)cc(F)c1]

Step 1: tert-butyl 2,4-dioxo-3-(5-(trifluoromethyl)pyrazin-2-yl)-1,3,8-triazaspiro[4.5]decane-8-carboxylate: Followed the general procedure I using tert-butyl 2,4-dioxo-1,3,8-triazaspiro[4.5]decane-8-carboxylate (200 mg, 0.743 mmol, 1.0 equiv.) and 2-chloro-6-(trifluoromethyl)pyrazine (163 mg, 0.891 mmol, 1.2 equiv.) as the starting materials to give tert-butyl 2,4-dioxo-3-(5-(trifluoromethyl)pyrazin-2-yl)-1,3,8-triazaspiro[4.5]decane-8-carboxylate (110 mg, 36%) as a white solid. MS m/z: 416 [M+H]⁺.

Step 2: tert-butyl 1-(3,5-difluorobenzyl)-2,4-dioxo-3-(5-(trifluoromethyl)pyrazin-2-yl)-1,3,8-triazaspiro[4.5]decane-8-carboxylate: Followed the general procedure K using tert-butyl 2,4-dioxo-3-(5-(trifluoromethyl)pyrazin-2-yl)-1,3,8-triazaspiro[4.5]decane-8-carboxylate (110 mg, 0.264 mmol, 1.0 equiv.) and 1-(bromomethyl)-3,5-difluorobenzene (65.5 mg, 0.317 mmol, 1.2 equiv.) as the starting materials to give tert-butyl 1-(3,5-difluorobenzyl)-2,4-dioxo-3-(5-(trifluoromethyl)pyrazin-2-yl)-1,3,8-triazaspiro[4.5]decane-8-carboxylate (90 mg, 62%) as a white solid. MS m/z: 542 [M+H]⁺.

Step 3: 1-(3,5-difluorobenzyl)-3-(5-(trifluoromethyl)pyrazin-2-yl)-1,3,8-triazaspiro[4.5]decane-2,4-dione hydrochloride: Followed the general procedure B using tert-butyl 1-(3,5-difluorobenzyl)-2,4-dioxo-3-(5-(trifluoromethyl)pyrazin-2-yl)-1,3,8-triazaspiro[4.5]decane-8-carboxylate (90 mg) as the starting material to give the crude product 1-(3,5-difluorobenzyl)-3-(5-(trifluoromethyl)pyrazin-2-yl)-1,3,8-triazaspiro[4.5]decane-2,4-dione hydrochloride (70 mg). MS m/z: 442 [M+H]⁺.

Step 4: 1-(3,5-difluorobenzyl)-8-(1-(2,2-difluoroethyl)-1H-pyrazolo[3,4-b]pyrazin-6-yl)-3-(5-(trifluoromethyl)pyrazin-2-yl)-1,3,8-triazaspiro[4.5]decane-2,4-dione: Followed the general procedure I using 1-(3,5-difluorobenzyl)-3-(5-(trifluoromethyl)pyrazin-2-yl)-1,3,8-triazaspiro[4.5]decane-2,4-dione hydrochloride (70 mg, 0.158 mmol, 1 equiv.) and 6-chloro-1-(2,2-difluoroethyl)-1H-pyrazolo[3,4-b]pyrazine (34.6 mg, 0.158 mmol, 1 equiv.) as the starting materials to give 1-(3,5-difluorobenzyl)-8-(1-(2,2-difluoroethyl)-1H-pyrazolo[3,4-b]pyrazin-6-yl)-3-(5-(trifluoromethyl)pyrazin-2-yl)-1,3,8-triazaspiro[4.5]decane-2,4-dione (54 mg, 54%) as a white solid. ¹H NMR (300 MHz, DMSO-d₆) δ 9.31 (s, 1H), 9.10 (s, 1H), 8.49 (s, 1H), 8.14 (s, 1H), 7.14 (d, J=7.9 Hz, 3H), 6.67-6.04 (m, 1H), 4.85-4.55 (m, 6H), 3.65-3.50 (m, 2H), 2.20-2.00 (m, 4H). MS m/z: 624.25 [M+H]⁺.

### Example 822: 8-(1-(2,2-difluoroethyl)-1H-pyrazolo[3,4-b]pyrazin-6-yl)-1-(2-ethoxyethyl)-3-(4-(trifluoromethyl)pyridin-2-yl)-1,3,8-triazaspiro[4.5]decane-2,4-dione

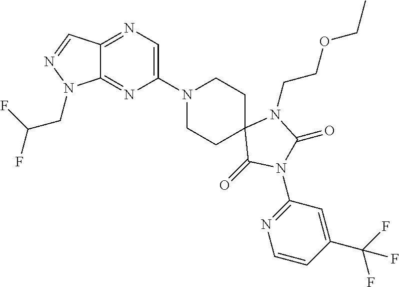 [SMILES: CCOCCN1C(=O)N(c2cc(C(F)(F)F)ccn2)C(=O)C12CCN(c1cnc3cnn(CC(F)F)c3n1)CC2]

Step 1: tert-butyl 1-(2-ethoxyethyl)-2,4-dioxo-3-(4-(trifluoromethyl)pyridin-2-yl)-1,3,8-triazaspiro[4.5]decane-8-carboxylate: Followed the general procedure K using tert-butyl 2,4-dioxo-3-(4-(trifluoromethyl)pyridin-2-yl)-1,3,8-triazaspiro[4.5]decane-8-carboxylate (60 mg, 0.145 mmol) and 1-bromo-2-ethoxyethane (26 mg, 0.174 mmol, 1.2 equiv.) as the starting materials to give tert-butyl 1-(2-ethoxyethyl)-2,4-dioxo-3-(4-(trifluoromethyl)pyridin-2-yl)-1,3,8-triazaspiro[4.5]decane-8-carboxylate (60 mg, 86%) as a white solid. MS m/z: 487 [M+H]⁺.

Step 2: 1-(2-ethoxyethyl)-3-(4-(trifluoromethyl)pyridin-2-yl)-1,3,8-triazaspiro[4.5]decane-2,4-dione hydrochloride: Followed the general procedure B using tert-butyl 1-(2-ethoxyethyl)-2,4-dioxo-3-(4-(trifluoromethyl)pyridin-2-yl)-1,3,8-triazaspiro[4.5]decane-8-carboxylate (60 mg, 0.123 mmol, 1 equiv) as the starting material to give the crude product 1-(2-ethoxyethyl)-3-(4-(trifluoromethyl)pyridin-2-yl)-1,3,8-triazaspiro[4.5]decane-2,4-dione hydrochloride (40 mg) MS m/z: 387 [M+H]⁺.

Step 3: 8-(1-(2,2-difluoroethyl)-1H-pyrazolo[3,4-b]pyrazin-6-yl)-1-(2-ethoxyethyl)-3-(4-(trifluoromethyl)pyridin-2-yl)-1,3,8-triazaspiro[4.5]decane-2,4-dione: Followed the general procedure I using 1-(2-ethoxyethyl)-3-(4-(trifluoromethyl)pyridin-2-yl)-1,3,8-triazaspiro[4.5]decane-2,4-dione hydrochloride (40 mg, 0.103 mmol, 1 equiv) and 6-chloro-1-(2,2-difluoroethyl)-1H-pyrazolo[3,4-b]pyrazine (27 mg, 0.124 mmol, 1.2 equiv) as the starting materials to give 8-(1-(2,2-difluoroethyl)-1H-pyrazolo[3,4-b]pyrazin-6-yl)-1-(2-ethoxyethyl)-3-(4-(trifluoromethyl)pyridin-2-yl)-1,3,8-triazaspiro[4.5]decane-2,4-dione (38 mg, 65%) as a white solid. ¹H NMR (400 MHz, DMSO-d₆) δ 8.91 (d, J=5.1 Hz, 1H), 8.53 (s, 1H), 8.16 (s, 1H), 8.02 (s, 1H), 7.93 (dd, J=5.0, 1.6 Hz, 1H), 6.45 (tt, J=54.9, 3.8 Hz, 1H), 4.78-4.59 (m, 4H), 3.63 (t, J=12.7 Hz, 2H), 3.55-3.48 (m, 2H), 3.48-3.37 (m, 4H), 2.25-2.13 (m, 2H), 2.10-2.02 (m, 2H), 1.04 (t, J=7.0 Hz, 3H). MS m/z: 569.15 [M+H]⁺.

### Example 823: 2-(6-(2-(2-(trifluoromethyl)pyrimidin-5-yl)-2,8-diazaspiro[4.5]decan-8-yl)pyrazin-2-yl)-1,3,4-thiadiazole

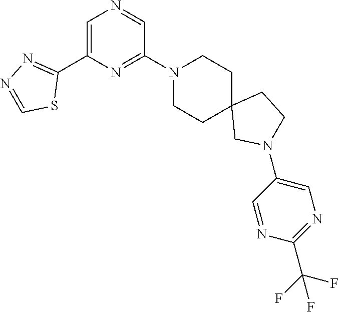 [SMILES: FC(F)(F)c1ncc(N2CCC3(CCN(c4cncc(-c5nncs5)n4)CC3)C2)cn1]

Step 1: tert-butyl 2-(2-(trifluoromethyl)pyrimidin-5-yl)-2,8-diazaspiro[4.5]decane-8-carboxylate: Followed the general procedure I using tert-butyl 2,8-diazaspiro[4.5]decane-8-carboxylate (100 mg, 0.42 mmol, 1.0 equiv.) and 5-bromo-2-(trifluoromethyl)pyrimidine (115 mg, 0.51 mmol, 1.1 equiv.) as the starting materials to give tert-butyl 2-(2-(trifluoromethyl)pyrimidin-5-yl)-2,8-diazaspiro[4.5]decane-8-carboxylate (80 mg, 49%) as a white solid. MS m/z: 387 [M+H]⁺.

Step 2: 2-(2-(trifluoromethyl)pyrimidin-5-yl)-2,8-diazaspiro[4.5]decane hydrochloride: Followed the general procedure B using tert-butyl 2-(2-(trifluoromethyl)pyrimidin-5-yl)-2,8-diazaspiro[4.5]decane-8-carboxylate (80 mg) as the starting material to give the crude product 2-(2-(trifluoromethyl)pyrimidin-5-yl)-2,8-diazaspiro[4.5]decane hydrochloride (80 mg). MS m/z: 287 [M+H]⁺.

Step 3: 2-(6-(2-(2-(trifluoromethyl)pyrimidin-5-yl)-2,8-diazaspiro[4.5]decan-8-yl)pyrazin-2-yl)-1,3,4-thiadiazole: Followed the general procedure I using 2-(6-chloropyrazin-2-yl)-1,3,4-thiadiazole (70 mg, 0.352 mmol, 1.0 equiv.) and 2-[2-(trifluoromethyl)pyrimidin-5-yl]-2,8-diazaspiro[4.5]decane (110 mg, 0.387 mmol, 1.1 equiv.) as the starting materials to give 2-(6-(2-(2-(trifluoromethyl)pyrimidin-5-yl)-2,8-diazaspiro[4.5]decan-8-yl)pyrazin-2-yl)-1,3,4-thiadiazole (90 mg, 56%) as a yellow green solid. ¹H NMR (400 MHz, DMSO-d₆) δ 9.71 (s, 1H), 8.62 (s, 1H), 8.56 (s, 1H), 8.24 (s, 2H), 3.83 (dt, J=11.9, 5.4 Hz, 2H), 3.71-3.63 (m, 2H), 3.50 (t, J=7.1 Hz, 2H), 3.38 (s, 2H), 1.98 (t, J=7.0 Hz, 2H), 1.71-1.66 (m, 4H). MS m/z: 449.05 [M+H]⁺

### Example 824: 2-(6-(2-(5-(trifluoromethyl)pyrazin-2-yl)-2,8-diazaspiro[4.5]decan-8-yl)pyrazin-2-yl)-1,3,4-thiadiazole

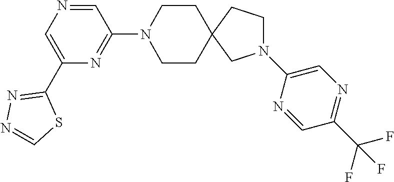 [SMILES: FC(F)(F)c1cnc(N2CCC3(CCN(c4cncc(-c5nncs5)n4)CC3)C2)cn1]

Step 1: tert-butyl 2-(5-(trifluoromethyl)pyrazin-2-yl)-2,8-diazaspiro[4.5]decane-8-carboxylate: Followed the general procedure I using tert-butyl 2,8-diazaspiro[4.5]decane-8-carboxylate (200 mg, 0.83 mmol, 1.0 equiv.) and 2-bromo-5-(trifluoromethyl)pyrazine (226 mg, 0.996 mmol, 1.2 equiv.) as the starting materials to give tert-butyl 2-(5-(trifluoromethyl)pyrazin-2-yl)-2,8-diazaspiro[4.5]decane-8-carboxylate (180 mg, 57%) as a white solid. MS m/z: 387 [M+H]⁺.

Step 2: 2-(5-(trifluoromethyl)pyrazin-2-yl)-2,8-diazaspiro[4.5]decane hydrochloride: Followed the general procedure B using tert-butyl 2-(5-(trifluoromethyl)pyrazin-2-yl)-2,8-diazaspiro[4.5]decane-8-carboxylate (180 mg) as the starting material to give the crude product 2-(5-(trifluoromethyl)pyrazin-2-yl)-2,8-diazaspiro[4.5]decane hydrochloride (120 mg). MS m/z: 287 [M+H]⁺.

Step 3: 2-(6-(2-(5-(trifluoromethyl)pyrazin-2-yl)-2,8-diazaspiro[4.5]decan-8-yl)pyrazin-2-yl)-1,3,4-thiadiazole: Followed the general procedure I using 2-(5-(trifluoromethyl)pyrazin-2-yl)-2,8-diazaspiro[4.5]decane hydrochloride (60 mg, 0.209 mmol, 1.0 equiv.) and 2-(6-chloropyrazin-2-yl)-1,3,4-thiadiazole (41.6 mg, 0.209 mmol, 1.0 equiv.) as the starting materials to give 2-(6-(2-(5-(trifluoromethyl)pyrazin-2-yl)-2,8-diazaspiro[4.5]decan-8-yl)pyrazin-2-yl)-1,3,4-thiadiazole (61 mg, 65%) as a yellow solid. ¹H NMR (400 MHz, DMSO-d₆) δ 9.71 (s, 1H), 8.62 (s, 1H), 8.55 (s, 1H), 8.46 (s, 1H), 8.09 (s, 1H), 3.84-3.77 (m, 2H), 3.71-3.63 (m, 4H), 3.51 (s, 2H), 1.98 (t, J=7.2 Hz, 2H), 1.72-1.65 (m, 4H). MS m/z: 449.05 [M+H]⁺.

### Example 825: 2-(6-(2-(6-(trifluoromethyl)pyridazin-3-yl)-2,8-diazaspiro[4.5]decan-8-yl)pyrazin-2-yl)-1,3,4-thiadiazole

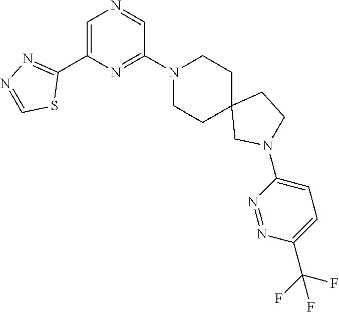 [SMILES: FC(F)(F)c1ccc(N2CCC3(CCN(c4cncc(-c5nncs5)n4)CC3)C2)nn1]

Step 1: 2-(6-(2-(6-(trifluoromethyl)pyridazin-3-yl)-2,8-diazaspiro[4.5]decan-8-yl)pyrazin-2-yl)-1,3,4-thiadiazole: Followed the general procedure I using 2-(6-(trifluoromethyl)pyridazin-3-yl)-2,8-diazaspiro[4.5]decane hydrochloride (100 mg, 0.31 mmol, 1.0 equiv.) and 2-(6-chloropyrazin-2-yl)-1,3,4-thiadiazole (67 mg, 0.341 mmol, 1.1 equiv.) as the starting materials to give 2-(6-(2-(6-(trifluoromethyl)pyridazin-3-yl)-2,8-diazaspiro[4.5]decan-8-yl)pyrazin-2-yl)-1,3,4-thiadiazole (80 mg, 57%) as a yellow green solid. ¹H NMR (400 MHz, DMSO-d₆) δ 9.80-9.58 (m, 1H), 8.76-8.38 (m, 2H), 7.85-7.68 (m, 1H), 7.10-6.91 (m, 1H), 3.86-3.47 (m, 8H), 2.03-1.96 (m, 2H), 1.76-1.62 (m, 4H). MS m/z: 449.2 [M+H]⁺.

### Example 826: 2-(6-(2-(2-(trifluoromethyl)pyrimidin-4-yl)-2,8-diazaspiro[4.5]decan-8-yl)pyridin-2-yl)-1,3,4-thiadiazole

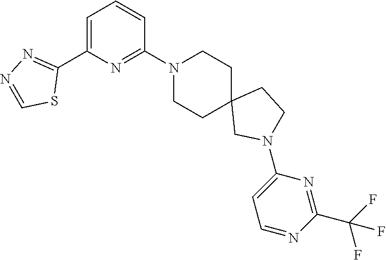 [SMILES: FC(F)(F)c1nccc(N2CCC3(CCN(c4cccc(-c5nncs5)n4)CC3)C2)n1]

Step 1: tert-butyl 2-(2-(trifluoromethyl)pyrimidin-4-yl)-2,8-diazaspiro[4.5]decane-8-carboxylate: Followed the general procedure I using tert-butyl 2,8-diazaspiro[4.5]decane-8-carboxylate (110 mg, 458 μmol, 1.0 equiv.) and 4-chloro-2-(trifluoromethyl)pyrimidine (84 mg, 458 μmol, 1.0 equiv.) as the starting materials to give 4-chloro-2-(trifluoromethyl)pyrimidine (120 mg, 67%) as a white solid. MS m/z: 387 [M+H]⁺.

Step 2: 2-(2-(trifluoromethyl)pyrimidin-4-yl)-2,8-diazaspiro[4.5]decane: Followed the general procedure H using 4-chloro-2-(trifluoromethyl)pyrimidine (120 mg) as the starting material to give the crude product 2-(2-(trifluoromethyl)pyrimidin-4-yl)-2,8-diazaspiro[4.5]decane (100 mg). MS m/z: 287 [M+H]⁺.

Step 3: 2-(6-(2-(2-(trifluoromethyl)pyrimidin-4-yl)-2,8-diazaspiro[4.5]decan-8-yl)pyridin-2-yl)-1,3,4-thiadiazole: Followed the general procedure I using 2-(2-(trifluoromethyl)pyrimidin-4-yl)-2,8-diazaspiro[4.5]decane (85 mg, 296 μmol, 1.0 equiv.) and 2-(6-chloropyrazin-2-yl)-1,3,4-thiadiazole (65 mg, 296 μmol, 1.0 equiv.) as the starting materials to give 2-(6-(2-(2-(trifluoromethyl)pyrimidin-4-yl)-2,8-diazaspiro[4.5]decan-8-yl)pyridin-2-yl)-1,3,4-thiadiazole (45 mg, 33%) as a white solid. ¹H NMR (400 MHz, DMSO-d₆) δ 9.71 (s, 1H), 8.62 (d, J=1.7 Hz, 1H), 8.55 (d, J=5.3 Hz, 1H), 8.30 (dd, J=6.2, 1.6 Hz, 1H), 6.73 (d, J=6.2 Hz, 1H), 3.85-3.60 (m, 5H), 3.53 (t, J=7.0 Hz, 3H), 2.01 (t, J=7.1 Hz, 1H), 1.93 (t, J=7.1 Hz, 1H), 1.68 (q, J=6.0, 4.7 Hz, 4H). MS m/z: 449.05 [M+H]⁺.

### Example 827: 8-(1-(2,2-difluoroethyl)-1H-pyrazolo[3,4-d]pyrimidin-6-yl)-2-(6-(trifluoromethyl)pyridin-3-yl)-2,8-diazaspiro[4.5]decane

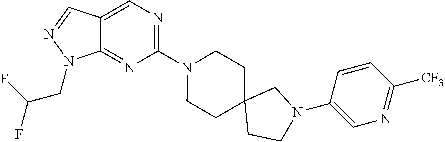 [SMILES: FC(F)Cn1ncc2cnc(N3CCC4(CCN(c5ccc(C(F)(F)F)nc5)C4)CC3)nc21]

Step 1: tert-butyl 2-(6-(trifluoromethyl)pyridin-3-yl)-2,8-diazaspiro[4.5]decane-8-carboxylate: Followed the general procedure I using tert-butyl 2,8-diazaspiro[4.5]decane-8-carboxylate (100 mg, 0.42 mmol, 1.0 equiv.) and 5-fluoro-2-(trifluoromethyl)pyridine (83 mg, 0.49 mmol, 1.1 equiv.) as the starting materials to give tert-butyl 2-(6-(trifluoromethyl)pyridin-3-yl)-2,8-diazaspiro[4.5]decane-8-carboxylate (80 mg, 51%) as a white solid. MS m/z: 386 [M+H]⁺.

Step 2: 2-(6-(trifluoromethyl)pyridin-3-yl)-2,8-diazaspiro[4.5]decane hydrochloride: Followed the general procedure B using tert-butyl 2-(6-(trifluoromethyl)pyridin-3-yl)-2,8-diazaspiro[4.5]decane-8-carboxylate (80 mg) as the starting material to give the crude product 2-(6-(trifluoromethyl)pyridin-3-yl)-2,8-diazaspiro[4.5]decane hydrochloride (80 mg). MS m/z: 286 [M+H]⁺.

Step 3: 8-(1-(2,2-difluoroethyl)-1H-pyrazolo[3,4-d]pyrimidin-6-yl)-2-(6-(trifluoromethyl)pyridin-3-yl)-2,8-diazaspiro[4.5]decane: Followed the general procedure I using 2-(6-(trifluoromethyl)pyridin-3-yl)-2,8-diazaspiro[4.5]decane hydrochloride (70 mg, 0.352 mmol, 1.0 equiv.) and 6-chloro-1-(2,2-difluoroethyl)-1H-pyrazolo[3,4-d]pyrimidine (110 mg, 0.387 mmol, 1.1 equiv.) as the starting materials to give 8-(1-(2,2-difluoroethyl)-1H-pyrazolo[3,4-d]pyrimidin-6-yl)-2-(6-(trifluoromethyl)pyridin-3-yl)-2,8-diazaspiro[4.5]decane (34.8 mg, 26%) as a white solid. ¹H NMR (400 MHz, DMSO-d₆) δ 8.91 (s, 1H), 8.09-8.02 (m, 2H), 7.59 (d, J=8.7 Hz, 1H), 6.99 (dd, J=8.8, 2.8 Hz, 1H), 6.44 (tt, J=54.9, 3.9 Hz, 1H), 4.67 (td, J=15.1, 3.8 Hz, 2H), 4.01 (dt, J=12.0, 5.0 Hz, 2H), 3.86 (dt, J=13.1, 5.0 Hz, 2H), 3.44 (t, J=6.9 Hz, 4H), 1.96 (t, J=6.9 Hz, 2H), 1.70-1.50 (m, 4H). MS m/z: 468.1 [M+H]⁺.

### Example 828: 8-(1-(2,2-difluoroethyl)-1H-pyrazolo[3,4-d]pyrimidin-6-yl)-2-(6-(trifluoromethyl)pyridin-3-yl)-2,8-diazaspiro[4.5]decan-3-one

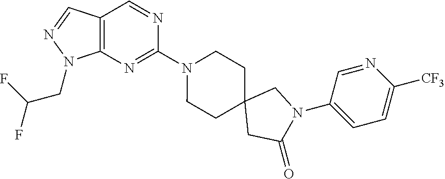 [SMILES: O=C1CC2(CCN(c3ncc4cnn(CC(F)F)c4n3)CC2)CN1c1ccc(C(F)(F)F)nc1]

Step 1: 8-(1-(2,2-difluoroethyl)-1H-pyrazolo[3,4-d]pyrimidin-6-yl)-2-(6-(trifluoromethyl)pyridin-3-yl)-2,8-diazaspiro[4.5]decan-3-one: Followed the general procedure I using 2-(6-(trifluoromethyl)pyridin-3-yl)-2,8-diazaspiro[4.5]decan-3-one hydrochloride (60.0 mg, 200 μmol, 1.2 equiv.) and 6-chloro-1-(2,2-difluoroethyl)-1H-pyrazolo[3,4-d]pyrimidine (36.5 mg, 167 μmol, 1.0 equiv.) as the starting materials to give 8-(1-(2,2-difluoroethyl)-1H-pyrazolo[3,4-d]pyrimidin-6-yl)-2-(6-(trifluoromethyl)pyridin-3-yl)-2,8-diazaspiro[4.5]decan-3-one (28.9 mg, 36%) as a yellow solid. ¹H NMR (400 MHz, DMSO-d₆) δ 9.06 (s, 1H), 8.92 (s, 1H), 8.44-8.38 (m, 1H), 8.08 (s, 1H), 7.94 (d, J=8.6 Hz, 1H), 6.64-6.27 (m, 1H), 4.82-4.57 (m, 2H), 4.08-4.01 (m, 2H), 3.91-3.80 (m, 4H), 2.64 (s, 2H), 1.71 (t, J=5.7 Hz, 4H). MS m/z: 482.05 [M+H]⁺.

### Example 829: 8-(1-(2,2-difluoroethyl)-3-methyl-1H-pyrazolo[3,4-b]pyrazin-6-yl)-2-(2-(trifluoromethyl)pyrimidin-5-yl)-2,8-diazaspiro[4.5]decane

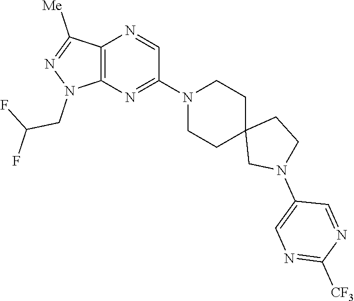 [SMILES: Cc1nn(CC(F)F)c2nc(N3CCC4(CCN(c5cnc(C(F)(F)F)nc5)C4)CC3)cnc12]

Step 1: 6-chloro-1-(2,2-difluoroethyl)-3-methyl-1H-pyrazolo[3,4-b]pyrazine: Followed the general procedure K using 6-chloro-3-methyl-1H-pyrazolo[3,4-b]pyrazine (300 mg, 1.79 mmol, 1.0 equiv.) and 2,2-difluoroethyl trifluoromethanesulfonate (573 mg, 2.69 mmol, 1.5 equiv.) as the starting materials to give 6-chloro-1-(2,2-difluoroethyl)-3-methyl-1H-pyrazolo[3,4-b]pyrazine (300 mg, 72%) as a white solid. MS m/z: 233 [M+H]⁺.

Step 2: 8-(1-(2,2-difluoroethyl)-3-methyl-1H-pyrazolo[3,4-b]pyrazin-6-yl)-2-(2-(trifluoromethyl)pyrimidin-5-yl)-2,8-diazaspiro[4.5]decane: A mixture of 6-chloro-1-(2,2-difluoroethyl)-3-methyl-1H-pyrazolo[3,4-b]pyrazine (70 mg, 301 μmol, 1.0 equiv.) and 2-(2-(trifluoromethyl)pyrimidin-5-yl)-2,8-diazaspiro[4.5]decane hydrochloride (129 mg, 452 μmol, 1.5 equiv.) as the starting materials to give 8-(1-(2,2-difluoroethyl)-3-methyl-1H-pyrazolo[3,4-b]pyrazin-6-yl)-2-(2-(trifluoromethyl)pyrimidin-5-yl)-2,8-diazaspiro[4.5]decane (70.6 mg, 47%) as a white solid. ¹H NMR (400 MHz, DMSO-d₆) δ 8.42 (s, 1H), 8.24 (s, 2H), 6.41 (tt, J=55.0, 3.9 Hz, 1H), 4.61 (td, J=15.0, 3.9 Hz, 2H), 3.95-3.86 (m, 2H), 3.78-3.67 (m, 2H), 3.50 (t, J=7.0 Hz, 2H), 3.38 (s, 2H), 2.42 (s, 3H), 1.97 (t, J=7.0 Hz, 2H), 1.74-1.60 (m, 4H). MS m/z: 509.15 [M+H]⁺.

### Example 830: 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decane

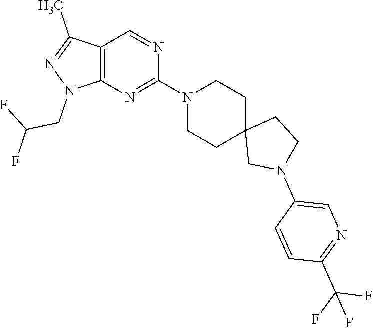 [SMILES: Cc1nn(CC(F)F)c2nc(N3CCC4(CCN(c5ccc(C(F)(F)F)nc5)C4)CC3)ncc12]

Step 1: 6-chloro-1-(2,2-difluoroethyl)-3-methyl-1H-pyrazolo[3,4-d]pyrimidine: Followed the general procedure K using 6-chloro-3-methyl-1H-pyrazolo[3,4-d]pyrimidine (150 mg, 0.89 mmol, 1.0 equiv.) and 2,2-difluoroethyl trifluoromethanesulfonate (285 mg, 1.34 mmol, 1.5 equiv.) as the starting materials to give 6-chloro-1-(2,2-difluoroethyl)-3-methyl-1H-pyrazolo[3,4-d]pyrimidine (40 mg, 20%) as a white solid. MS m/z: 387 [M+H]⁺.

Step 2: 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decane: Followed the general procedure I using 6-chloro-1-(2,2-difluoroethyl)-3-methyl-1H-pyrazolo[3,4-d]pyrimidine (30 mg, 0.129 mmol, 1.0 equiv.) and 2-(6-(trifluoromethyl)pyridin-3-yl)-2,8-diazaspiro[4.5]decane (44 mg, 0.155 mmol, 1.2 equiv.) as the starting materials to give 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decane (13.1 mg, 21%) as a white solid. ¹H NMR (400 MHz, DMSO-d₆) δ 8.88 (s, 1H), 8.09-8.02 (m, 1H), 7.60 (d, J=8.7 Hz, 1H), 7.00 (dd, J=8.9, 2.5 Hz, 1H), 6.41 (t, J=55.0 Hz, 1H), 4.63-4.51 (m, 2H), 4.00 (s, 2H), 3.85 (s, 2H), 3.45 (d, J=7.1 Hz, 2H), 3.35-3.37 (m, 2H), 2.42 (s, 3H), 1.97 (t, J=7.0 Hz, 2H), 1.62 (s, 4H). MS m/z: 482.05 [M+H]⁺

### Example 831: 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decan-1-one

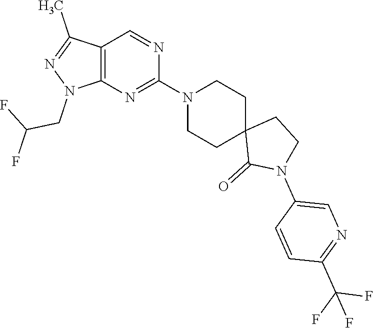 [SMILES: Cc1nn(CC(F)F)c2nc(N3CCC4(CC3)CCN(c3ccc(C(F)(F)F)nc3)C4=O)ncc12]

8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decan-1-one: Followed the general procedure I using 6-chloro-1-(2,2-difluoroethyl)-3-methyl-1H-pyrazolo[3,4-d]pyrimidine (50 mg, 0.252 mmol, 1.0 equiv.) and 2-(6-(trifluoromethyl)pyridin-3-yl)-2,8-diazaspiro[4.5]decan-1-one (77 mg, 0.258 mmol, 1.2 equiv.) as the starting materials to give 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decan-1-one (20.7 mg, 19%) as a white solid. ¹H NMR (400 MHz, DMSO-d₆) δ 9.09 (d, J=2.5 Hz, 1H), 8.90 (s, 1H), 8.42 (dd, J=8.6, 2.4 Hz, 1H), 7.94 (d, J=8.6 Hz, 1H), 6.59-6.24 (m, 1H), 4.71-4.53 (m, 4H), 3.97 (t, J=6.9 Hz, 2H), 3.27-3.32 (m, 2H), 2.43 (s, 3H), 2.26 (t, J=6.9 Hz, 2H), 1.79-1.69 (m, 2H), 1.66 (d, J=13.5 Hz, 2H). MS m/z: 496.25 [M+H]⁺.

### Example 832: 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decan-3-one

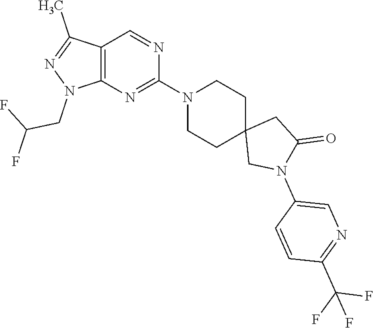 [SMILES: Cc1nn(CC(F)F)c2nc(N3CCC4(CC3)CC(=O)N(c3ccc(C(F)(F)F)nc3)C4)ncc12]

Step 1: 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decan-3-one: Followed the general procedure I using 6-chloro-1-(2,2-difluoroethyl)-3-methyl-1H-pyrazolo[3,4-d]pyrimidine (50 mg, 0.252 mmol, 1.0 equiv.) and 2-(6-(trifluoromethyl)pyridin-3-yl)-2,8-diazaspiro[4.5]decan-3-one (90 mg, 0.302 mmol, 1.2 equiv.) as the starting materials to give 8-(1-(2,2-difluoroethyl)-3-methyl-1H-pyrazolo[3,4-d]pyrimidin-6-yl)-2-(6-(trifluoromethyl)pyridin-3-yl)-2,8-diazaspiro[4.5]decan-3-one (71.9 mg, 57%) as a white solid. ¹H NMR (400 MHz, DMSO-d₆) δ 9.05 (d, J=2.5 Hz, 1H), 8.88 (s, 1H), 8.40 (dd, J=8.8, 2.5 Hz, 1H), 7.94 (d, J=8.8 Hz, 1H), 6.56-6.21 (m, 1H), 4.58 (td, J=15.0, 3.8 Hz, 2H), 4.09-4.00 (m, 2H), 3.85 (d, J=10.7 Hz, 4H), 2.63 (s, 2H), 2.42 (s, 3H), 1.69 (t, J=5.7 Hz, 4H). MS m/z: 496.15 [M+H]⁺.

### Example 833: 8-(1-(2,2-difluoroethyl)-3-methoxy-1H-pyrazolo[3,4-b]pyrazin-6-yl)-2-(6-(trifluoromethyl)pyridin-3-yl)-2,8-diazaspiro[4.5]decane

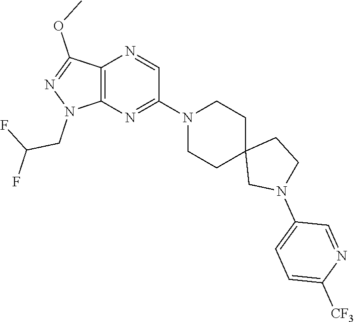 [SMILES: COc1nn(CC(F)F)c2nc(N3CCC4(CCN(c5ccc(C(F)(F)F)nc5)C4)CC3)cnc12]

Step 1: tert-butyl 2-(6-(trifluoromethyl)pyridin-3-yl)-2,8-diazaspiro[4.5]decane-8-carboxylate: Followed the general procedure I using tert-butyl 2,8-diazaspiro[4.5]decane-8-carboxylate (300 mg, 1.24 mmol, 1.0 equiv.) and 5-fluoro-2-(trifluoromethyl)pyridine (248 mg, 1.49 mmol, 1.2 equiv.) as the starting materials to give tert-butyl 2-(6-(trifluoromethyl)pyridin-3-yl)-2,8-diazaspiro[4.5]decane-8-carboxylate (400 mg, 83.2%) as a light yellow oil. MS m/z: 386 [M+H]⁺.

Step 2: 2-(6-(trifluoromethyl)pyridin-3-yl)-2,8-diazaspiro[4.5]decane hydrochloride: Followed the general procedure B using tert-butyl 2-(6-(trifluoromethyl)pyridin-3-yl)-2,8-diazaspiro[4.5]decane-8-carboxylate (400 mg, 1.04 mmol, 1.0 equiv.) as the starting material to give the crude product 2-(6-(trifluoromethyl)pyridin-3-yl)-2,8-diazaspiro[4.5]decane hydrochloride (300 mg) as a light yellow solid. MS m/z: 286 [M+H]⁺

Step 3: 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decane: Followed the general procedure I using 2-(6-(trifluoromethyl)pyridin-3-yl)-2,8-diazaspiro[4.5]decane hydrochloride (150 mg, 0.524 mmol, 1.0 equiv.) and 6-chloro-3-iodo-1-(tetrahydro-2H-pyran-2-yl)-1H-pyrazolo[3,4-b]pyrazine (230 mg, 0.629 mmol, 1.2 equiv.) as the starting materials to give 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decane (150 mg, 46.6%) as a light yellow oil. MS m/z: 614 [M+H]⁺.

Step 4: 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decane: Followed the general procedure J using 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decane (150 mg, 0.244 mmol, 1.0 equiv.) and MeOH (0.2 mL) as the starting materials to give 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decane (120 mg, 94.8%) as a white solid. MS m/z: 518 [M+H]⁺.

Step 5: 8-(3-methoxy-1H-pyrazolo[3,4-b]pyrazin-6-yl)-2-(6-(trifluoromethyl)pyridin-3-yl)-2,8-diazaspiro[4.5]decane: Followed the general procedure M using 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decane (120 mg, 0.232 mmol, 1.0 equiv.) as the starting material to give 8-(3-methoxy-1H-pyrazolo[3,4-b]pyrazin-6-yl)-2-(6-(trifluoromethyl)pyridin-3-yl)-2,8-diazaspiro[4.5]decane (90 mg, 89.5%) as a light yellow solid. MS m/z: 434 [M+H]⁺

Step 6: 8-(1-(2,2-difluoroethyl)-3-methoxy-1H-pyrazolo[3,4-b]pyrazin-6-yl)-2-(6-(trifluoromethyl)pyridin-3-yl)-2,8-diazaspiro[4.5]decane: Followed the general procedure K using 8-(3-methoxy-1H-pyrazolo[3,4-b]pyrazin-6-yl)-2-(6-(trifluoromethyl)pyridin-3-yl)-2,8-diazaspiro[4.5]decane (90 mg, 0.207 mmol, 1.0 equiv.) and 2,2-difluoroethyl trifluoromethanesulfonate (49.0 mg, 0.228 mmol, 1.1 equiv.) as the starting materials to give 8-(1-(2,2-difluoroethyl)-3-methoxy-1H-pyrazolo[3,4-b]pyrazin-6-yl)-2-(6-(trifluoromethyl)pyridin-3-yl)-2,8-diazaspiro[4.5]decane (58.8 mg, 56.8%) as a white solid. ¹H NMR (400 MHz, DMSO-d₆) δ 8.35 (s, 1H), 8.04 (d, 1H), 7.59 (d, 1H), 7.00 (dd, 1H), 6.37 (tt, 1H), 4.51 (td, 2H), 3.99 (s, 3H), 3.88 (dt, 5.2 Hz, 2H), 3.75 (dt, 2H), 3.45 (t, 2H), 3.35 (t, 2H), 1.97 (t, 2H), 1.66 (dt, 4H). MS m/z: 498.15 [M+H]⁺.

### Example 834: 8-(1-(2,2-difluoroethyl)-5-methoxy-1H-pyrazolo[3,4-b]pyrazin-6-yl)-2-(6-(trifluoromethyl)pyridin-3-yl)-2,8-diazaspiro[4.5]decan-1-one

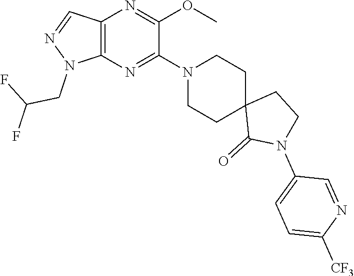 [SMILES: COc1nc2cnn(CC(F)F)c2nc1N1CCC2(CC1)CCN(c1ccc(C(F)(F)F)nc1)C2=O]

Step 1: 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-1-one: Followed the general procedure I using 2-(6-(trifluoromethyl)pyridin-3-yl)-2,8-diazaspiro[4.5]decan-1-one (100 mg, 0.334 mmol, 1 equiv) and 6-chloro-1-(2,2-difluoroethyl)-1H-pyrazolo[3,4-b]pyrazine (87 mg, 0.401 mmol, 1.2 equiv) as the starting materials to give 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-1-one (125 mg, 78%) as a white solid. MS m/z: 482 [M+H]⁺.

Step 2: 8-(1-(2,2-difluoroethyl)-3-fluoro-1H-pyrazolo[3,4-b]pyrazin-6-yl)-2-(6-(trifluoromethyl)pyridin-3-yl)-2,8-diazaspiro[4.5]decan-1-one: A mixture of 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-1-one (115 mg, 0.239 mmol, 1 equiv) and Selectfluor (126 mg, 0.358 mmol, 1.5 equiv) in MeCN was stirred for overnight at 80° C. under air atmosphere. The resulting mixture was concentrated under reduced pressure. The residue was purified by reversed-phase flash chromatography with the following conditions: column, C18 silica gel; mobile phase, MeCN in Water (0.1% FA), 0% to 100% gradient in 30 min; detector, UV 254 nm. The resulting mixture was concentrated under reduced pressure. This resulted in 8-(1-(2,2-difluoroethyl)-3-fluoro-1H-pyrazolo[3,4-b]pyrazin-6-yl)-2-(6-(trifluoromethyl)pyridin-3-yl)-2,8-diazaspiro[4.5]decan-1-one (100 mg, 83%) as a white solid. MS m/z: 500 [M+H]⁺.

Step 3: 8-(1-(2,2-difluoroethyl)-5-methoxy-1H-pyrazolo[3,4-b]pyrazin-6-yl)-2-(6-(trifluoromethyl)pyridin-3-yl)-2,8-diazaspiro[4.5]decan-1-one: A solution of 8-(1-(2,2-difluoroethyl)-3-fluoro-1H-pyrazolo[3,4-b]pyrazin-6-yl)-2-(6-(trifluoromethyl)pyridin-3-yl)-2,8-diazaspiro[4.5]decan-1-one (100 mg, 0.200 mmol, 1 equiv) and sodium methanolate (32.4 mg, 0.601 mmol, 3.00 equiv) in MeOH was stirred for 2 h at 60° C. under air atmosphere. The resulting mixture was concentrated under reduced pressure. The residue was purified by reverse flash chromatography with the following conditions: column, C18 silica gel; mobile phase, MeCN in Water (0.1% FA), 0% to 100% gradient in 30 min; detector, UV 254 nm. This resulted in 8-(1-(2,2-difluoroethyl)-5-methoxy-1H-pyrazolo[3,4-b]pyrazin-6-yl)-2-(6-(trifluoromethyl)pyridin-3-yl)-2,8-diazaspiro[4.5]decan-1-one (23 mg, 22%) as a white solid. ¹H NMR (400 MHz, DMSO-d₆) δ 9.09 (d, J=2.5 Hz, 1H), 8.43 (dd, J=8.7, 2.6 Hz, 1H), 8.07-7.87 (m, 2H), 6.44 (tt, J=55.0, 3.9 Hz, 1H), 4.71 (td, J=15.0, 3.9 Hz, 2H), 4.26 (dt, J=13.6, 4.0 Hz, 2H), 4.06-3.90 (m, 5H), 3.30-3.19 (m, 2H), 2.25 (t, J=6.9 Hz, 2H), 1.99-1.87 (m, 2H), 1.74-1.67 (m, 2H). MS m/z: 512.05 [M+H]⁺.

### Example 835: 8-(1-(2,2-difluoroethyl)-3-methoxy-1H-pyrazolo[3,4-b]pyrazin-6-yl)-2-(6-(trifluoromethyl)pyridin-3-yl)-2,8-diazaspiro[4.5]decan-1-one

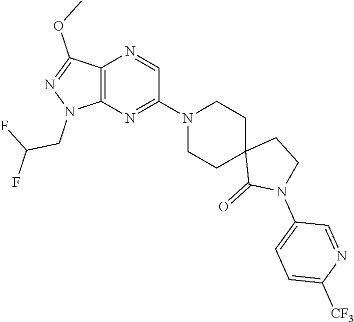 [SMILES: COc1nn(CC(F)F)c2nc(N3CCC4(CC3)CCN(c3ccc(C(F)(F)F)nc3)C4=O)cnc12]

Step 1: tert-butyl 1-oxo-2-(6-(trifluoromethyl)pyridin-3-yl)-2,8-diazaspiro[4.5]decane-8-carboxylate: Followed the general procedure Y using tert-butyl 1-oxo-2,8-diazaspiro[4.5]decane-8-carboxylate (300 mg, 1.18 mmol, 1.0 equiv.) and 5-bromo-2-(trifluoromethyl)pyridine (320 mg, 1.42 mmol, 1.2 equiv.) as the starting materials, RuPhos Pd G3 (98.7 mg, 0.118 mmol, 0.1 equiv.) as the catalyst to give tert-butyl 1-oxo-2-(6-(trifluoromethyl)pyridin-3-yl)-2,8-diazaspiro[4.5]decane-8-carboxylate (420 mg, 89%) as a white solid. MS m/z: 399 [M+H]⁺.

Step 2: 2-(6-(trifluoromethyl)pyridin-3-yl)-2,8-diazaspiro[4.5]decan-1-one hydrochloride: Followed the general procedure B using tert-butyl 1-oxo-2-(6-(trifluoromethyl)pyridin-3-yl)-2,8-diazaspiro[4.5]decane-8-carboxylate (410 mg) as the starting material to give the crude product 2-(6-(trifluoromethyl)pyridin-3-yl)-2,8-diazaspiro[4.5]decan-1-one hydrochloride (280 mg). MS m/z: 299 [M+H]⁺.

Step 3: 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-1-one: Followed the general procedure I using 2-(6-(trifluoromethyl)pyridin-3-yl)-2,8-diazaspiro[4.5]decan-1-one (270 mg, 0.902 mmol, 1.0 equiv.) and 6-chloro-3-iodo-1-(tetrahydro-2H-pyran-2-yl)-1H-pyrazolo[3,4-b]pyrazine (362 mg, 0.992 mmol, 1.1 equiv.) as the starting materials to give 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-1-one (510 mg, 90%) as a yellow solid. MS m/z: 627 [M+H]⁺.

Step 4: 3,5-dimethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure J using 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-1-one (200 mg, 0.319 mmol, 1.0 equiv.) and MeOH (25 mg, 0.781 mmol, 2.0 equiv.) as the starting materials to give 3,5-dimethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (75 mg, 44%) as a yellow oil. MS m/z: 531 [M+H]⁺.

Step 5: 8-(3-methoxy-1H-pyrazolo[3,4-b]pyrazin-6-yl)-2-(6-(trifluoromethyl)pyridin-3-yl)-2,8-diazaspiro[4.5]decan-1-one: Followed the general procedure M using 3,5-dimethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (75 mg, 0.141 mmol, 1 equiv.) as the starting material to give 8-(3-methoxy-1H-pyrazolo[3,4-b]pyrazin-6-yl)-2-(6-(trifluoromethyl)pyridin-3-yl)-2,8-diazaspiro[4.5]decan-1-one (40 mg, 63%) as a white solid. MS m/z: 447 [M+H]⁺.

Step 6: 8-(1-(2,2-difluoroethyl)-3-ethoxy-1H-pyrazolo[3,4-b]pyrazin-6-yl)-2-(6-(trifluoromethyl)pyridin-3-yl)-2,8-diazaspiro[4.5]decan-3-one: Followed the general procedure K using 8-(3-methoxy-1H-pyrazolo[3,4-b]pyrazin-6-yl)-2-(6-(trifluoromethyl)pyridin-3-yl)-2,8-diazaspiro[4.5]decan-1-one (30 mg, 0.067 mmol, 1 equiv.) and 2,2-difluoroethyl trifluoromethanesulfonate (21.5 mg, 0.101 mmol, 1.5 equiv.) as the starting materials to give 8-(1-(2,2-difluoroethyl)-3-ethoxy-1H-pyrazolo[3,4-b]pyrazin-6-yl)-2-(6-(trifluoromethyl)pyridin-3-yl)-2,8-diazaspiro[4.5]decan-3-one (18.2 mg, 53%) as a white solid. ¹H NMR (400 MHz, DMSO-d₆) δ 9.09 (d, J=2.5 Hz, 1H), 8.42 (d, J=9.0 Hz, 1H), 8.37 (s, 1H), 7.94 (d, J=8.7 Hz, 1H), 6.37 (tt, J=55.1 Hz, 3.5 Hz, 1H), 4.61-4.48 (m, 2H), 4.48-4.34 (m, 4H), 4.00-3.91 (m, 5H), 2.27-2.17 (m, 2H), 1.85-1.78 (m, 2H), 1.74-1.62 (m, 2H). MS m/z: 512.20 [M+H]⁺.

### Example 836: 8-(1-(2,2-difluoroethyl)-5-methoxy-1H-pyrazolo[3,4-b]pyrazin-6-yl)-2-(6-(trifluoromethyl)pyridin-3-yl)-2,8-diazaspiro[4.5]decan-3-one

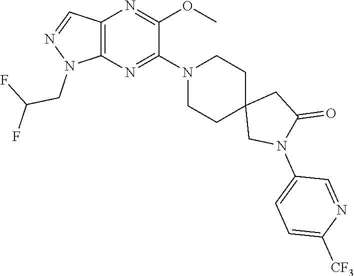 [SMILES: COc1nc2cnn(CC(F)F)c2nc1N1CCC2(CC1)CC(=O)N(c1ccc(C(F)(F)F)nc1)C2]

Step 1: 8-(1-(2,2-difluoroethyl)-5-methoxy-1H-pyrazolo[3,4-b]pyrazin-6-yl)-2-(6-(trifluoromethyl)pyridin-3-yl)-2,8-diazaspiro[4.5]decan-3-one: A solution of 8-(1-(2,2-difluoroethyl)-1H-pyrazolo[3,4-b]pyrazin-6-yl)-2-(6-(trifluoromethyl)pyridin-3-yl)-2,8-diazaspiro[4.5]decan-3-one (100 mg, 0.208 mmol, 1.0 equiv.) and Selectfluor (110 mg, 0.312 mmol, 1.5 equiv.) in ACN (5 mL) was stirred for overnight at 80° C. The resulting mixture was concentrated under vacuum. Then, The residue was dissolved in MeOH (5 mL). The resulting mixture were added CH₃ONa (33.7 mg, 0.624 mmol, 3.0 equiv.) was stirred for 2 h at 60° C. The resulting mixture was concentrated under vacuum. The residue was purified by silica gel column chromatography, eluted with PE/EA (1:1.5) to afford 8-(1-(2,2-difluoroethyl)-5-methoxy-1H-pyrazolo[3,4-b]pyrazin-6-yl)-2-(6-(trifluoromethyl)pyridin-3-yl)-2,8-diazaspiro[4.5]decan-3-one. ¹H NMR (400 MHz, DMSO-d₆) δ 9.06 (d, J=2.5 Hz, 1H), 8.41 (dd, J=8.7, 2.5 Hz, 1H), 8.03-7.86 (m, 2H), 6.45 (tt, J=54.9, 3.9 Hz, 1H), 4.71 (td, J=14.9, 3.9 Hz, 2H), 3.97 (s, 3H), 3.88 (s, 2H), 3.83-3.70 (m, 2H), 3.69-3.53 (m, 2H), 2.63 (s, 2H), 1.85-1.74 (m, 4H). MS m/z: 512.1 [M+H]⁺.

### Example 837: 8-(1-(2,2-difluoroethyl)-3-methoxy-1H-pyrazolo[3,4-b]pyrazin-6-yl)-2-(6-(trifluoromethyl)pyridin-3-yl)-2,8-diazaspiro[4.5]decan-3-one

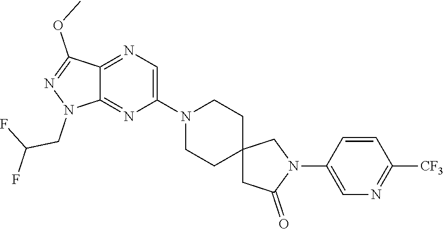 [SMILES: COc1nn(CC(F)F)c2nc(N3CCC4(CC3)CC(=O)N(c3ccc(C(F)(F)F)nc3)C4)cnc12]

Step 1: 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one: Followed the general procedure I using 2-(6-(trifluoromethyl)pyridin-3-yl)-2,8-diazaspiro[4.5]decan-3-one hydrochloride (390 mg, 1.30 mmol, 1.0 equiv.) and 6-chloro-3-iodo-1-(tetrahydro-2H-pyran-2-yl)-1H-pyrazolo[3,4-b]pyrazine (570 mg, 1.56 mmol, 1.2 equiv.) as the starting materials to give 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (500 mg, 61%) as a white solid. MS m/z: 628 [M+H].

Step 2: 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one: Followed the general procedure J using 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (240 mg, 0.383 mmol, 1.0 equiv.) and MeOH (25 mg, 0.781 mmol, 2.0 equiv.) as the starting materials to give 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (60 mg, 30%) as a white solid. MS m/z: 532 [M+H]⁺.

Step 3: 8-(3-methoxy-1H-pyrazolo[3,4-b]pyrazin-6-yl)-2-(6-(trifluoromethyl)pyridin-3-yl)-2,8-diazaspiro[4.5]decan-3-one: Followed the general procedure M using 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (50 mg, 0.094 mmol, 1.0 equiv.) as the starting material to give 8-(3-methoxy-1H-pyrazolo[3,4-b]pyrazin-6-yl)-2-(6-(trifluoromethyl)pyridin-3-yl)-2,8-diazaspiro[4.5]decan-3-one (30 mg, 71%) as a white solid. MS m/z: 448 [M+H]⁺.

Step 4: 8-(1-(2,2-difluoroethyl)-3-methoxy-1H-pyrazolo[3,4-b]pyrazin-6-yl)-2-(6-(trifluoromethyl)pyridin-3-yl)-2,8-diazaspiro[4.5]decan-3-one: Followed the general procedure K using 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (40.0 mg, 0.090 mmol, 1.0 equiv.) and 2,2-difluoroethyl trifluoromethanesulfonate (21.3 mg, 0.099 mmol, 1.1 equiv.) as the starting materials to give 8-(1-(2,2-difluoroethyl)-3-methoxy-1H-pyrazolo[3,4-b]pyrazin-6-yl)-2-(6-(trifluoromethyl)pyridin-3-yl)-2,8-diazaspiro[4.5]decan-3-one (23 mg, 98%) as a white solid. ¹H NMR (300 MHz, DMSO-d₆) δ 9.05 (d, J=2.5 Hz, 1H), 8.48-8.19 (m, 2H), 7.94 (d, J=8.8 Hz, 1H), 6.40 (tt, 1H), 4.51 (m, 2H), 3.99 (s, 3H), 3.98-3.81 (m, 4H), 3.80-3.71 (m, 2H), 2.64 (s, 2H), 1.75 (d, J=5.9 Hz, 4H). MS m/z: 512.15[M+H]⁺.

### Example 838: 8-(1-(2,2-difluoroethyl)-1H-pyrazolo[3,4-b]pyrazin-6-yl)-2-(4-(trifluoromethyl)oxazol-2-yl)-2,8-diazaspiro[4.5]decan-1-one

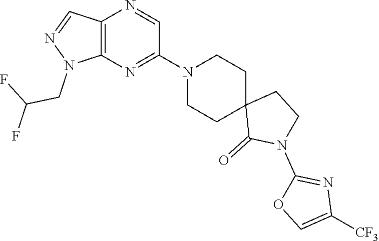 [SMILES: O=C1N(c2nc(C(F)(F)F)co2)CCC12CCN(c1cnc3cnn(CC(F)F)c3n1)CC2]

Step 1: ethyl 2-(8-(tert-butoxycarbonyl)-1-oxo-2,8-diazaspiro[4.5]decan-2-yl)-4-(trifluoromethyl)oxazole-5-carboxylate: Followed the general procedure I using tert-butyl 1-oxo-2,8-diazaspiro[4.5]decane-8-carboxylate (500 mg, 1.96 mmol, 1.0 equiv.) and ethyl 2-chloro-4-(trifluoromethyl)oxazole-5-carboxylate (530 mg, 2.16 mmol, 1.1 equiv.) as the starting materials to give ethyl 2-(8-(tert-butoxycarbonyl)-1-oxo-2,8-diazaspiro[4.5]decan-2-yl)-4-(trifluoromethyl)oxazole-5-carboxylate (260 mg, 28%) as a white solid. MS m/z: 462 [M+H]⁺.

Step 2: ethyl 2-(1-oxo-2,8-diazaspiro[4.5]decan-2-yl)-4-(trifluoromethyl)oxazole-5-carboxylate hydrochloride: Followed the general procedure B using ethyl 2-(8-(tert-butoxycarbonyl)-1-oxo-2,8-diazaspiro[4.5]decan-2-yl)-4-(trifluoromethyl)oxazole-5-carboxylate (260 mg) as the starting material to give the crude product ethyl 2-(1-oxo-2,8-diazaspiro[4.5]decan-2-yl)-4-(trifluoromethyl)oxazole-5-carboxylate hydrochloride (120 mg). MS m/z: 362 [M+H]⁺.

Step 3: 8-(1-(2,2-difluoroethyl)-1H-pyrazolo[3,4-b]pyrazin-6-yl)-2-(4-(trifluoromethyl)oxazol-2-yl)-2,8-diazaspiro[4.5]decan-1-one: Followed the general procedure I using ethyl 2-(1-oxo-2,8-diazaspiro[4.5]decan-2-yl)-4-(trifluoromethyl)oxazole-5-carboxylate hydrochloride (120 mg, 0.332 mmol, 1.0 equiv.) and 6-chloro-1-(2,2-difluoroethyl)-1H-pyrazolo[3,4-b]pyrazine (80 mg, 0.365 mmol, 1.1 equiv.) as the starting materials to give 8-(1-(2,2-difluoroethyl)-1H-pyrazolo[3,4-b]pyrazin-6-yl)-2-(4-(trifluoromethyl)oxazol-2-yl)-2,8-diazaspiro[4.5]decan-1-one (24 mg, 14%) as a white solid. ¹H NMR (400 MHz, DMSO-d₆) δ 8.86-8.66 (m, 1H), 8.51 (s, 1H), 8.15 (s, 1H), 6.76-6.16 (m, 1H), 4.71 (td, J=15.0, 3.8 Hz, 2H), 4.53-4.07 (m, 2H), 3.96 (t, J=7.0 Hz, 2H), 3.41-3.33 (m, 2H), 2.24 (t, J=7.0 Hz, 2H), 1.91-0.78 (m, 4H). MS m/z: 472 [M+H]⁺.

### Example 839: 8-(1-(2,2-difluoroethyl)-1H-pyrazolo[3,4-b]pyrazin-6-yl)-2-(5-(trifluoromethyl)-1,3,4-oxadiazol-2-yl)-2,8-diazaspiro[4.5]decan-1-one

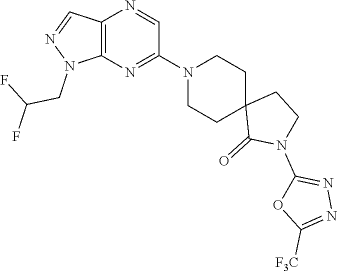 [SMILES: O=C1N(c2nnc(C(F)(F)F)o2)CCC12CCN(c1cnc3cnn(CC(F)F)c3n1)CC2]

Step 1: tert-butyl 1-oxo-2-(5-(trifluoromethyl)-1,3,4-oxadiazol-2-yl)-2,8-diazaspiro[4.5]decane-8-carboxylate: Followed the general procedure Y using tert-butyl 1-oxo-2,8-diazaspiro[4.5]decane-8-carboxylate (259 mg, 1013 μmol, 1.0 equiv.) and 2-bromo-5-(trifluoromethyl)-1,3,4-oxadiazole (200 mg, 921 μmol, 1.0 equiv.) as the starting materials, Ruphos Pd G3 (77 mg, 92 μmol, 0.1 equiv.) as the catalyst to give tert-butyl 1-oxo-2-(5-(trifluoromethyl)-1,3,4-oxadiazol-2-yl)-2,8-diazaspiro[4.5]decane-8-carboxylate (80 mg, 22%) as a white solid. MS m/z: 391 [M+H]⁺.

Step 2: 2-(5-(trifluoromethyl)-1,3,4-oxadiazol-2-yl)-2,8-diazaspiro[4.5]decan-1-one: Followed the general procedure H using tert-butyl 1-oxo-2-(5-(trifluoromethyl)-1,3,4-oxadiazol-2-yl)-2,8-diazaspiro[4.5]decane-8-carboxylate (80 mg) as the starting material to give the crude product 2-(5-(trifluoromethyl)-1,3,4-oxadiazol-2-yl)-2,8-diazaspiro[4.5]decan-1-one (60 mg). MS m/z: 291 [M+H]⁺.

Step 3: 8-(1-(2,2-difluoroethyl)-1H-pyrazolo[3,4-b]pyrazin-6-yl)-2-(5-(trifluoromethyl)-1,3,4-oxadiazol-2-yl)-2,8-diazaspiro[4.5]decan-1-one: Followed the general procedure I using 2-(5-(trifluoromethyl)-1,3,4-oxadiazol-2-yl)-2,8-diazaspiro[4.5]decan-1-one (60 mg, 206 μmol, 1.0 equiv.) and 6-chloro-1-(2,2-difluoroethyl)-1H-pyrazolo[3,4-b]pyrazine (50 mg, 226 μmol, 1.1 equiv.) as the starting materials to give 8-(1-(2,2-difluoroethyl)-1H-pyrazolo[3,4-b]pyrazin-6-yl)-2-(5-(trifluoromethyl)-1,3,4-oxadiazol-2-yl)-2,8-diazaspiro[4.5]decan-1-one (19.1 mg, 19%) as a white solid. ¹H NMR (400 MHz, DMSO-d₆) δ 8.52 (s, 1H), 8.15 (d, J=1.1 Hz, 1H), 6.58-6.31 (m, 1H), 4.71 (td, J=15.1, 3.8 Hz, 2H), 4.37 (dt, J=13.9, 4.4 Hz, 2H), 4.01 (t, J=7.0 Hz, 2H), 3.42 (s, 2H), 2.30 (t, J=7.1 Hz, 2H), 1.89-1.70 (m, 4H). MS m/z: 473.20 [M+H]⁺.

### Example 840: 8-(1-(2,2-difluoroethyl)-1H-pyrazolo[3,4-b]pyrazin-6-yl)-2-(5-(trifluoromethyl)-1,3,4-thiadiazol-2-yl)-2,8-diazaspiro[4.5]decan-1-one

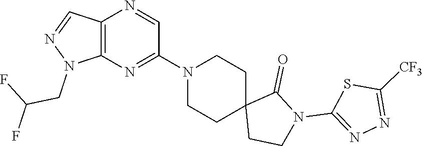 [SMILES: O=C1N(c2nnc(C(F)(F)F)s2)CCC12CCN(c1cnc3cnn(CC(F)F)c3n1)CC2]

Step 1: tert-butyl 1-oxo-2-(5-(trifluoromethyl)-1,3,4-thiadiazol-2-yl)-2,8-diazaspiro[4.5]decane-8-carboxylate: Followed the general procedure Y using tert-butyl 1-oxo-2,8-diazaspiro[4.5]decane-8-carboxylate (100 mg, 392 μmol, 1.0 equiv.) and 2-chloro-5-(trifluoromethyl)-1,3,4-thiadiazole (82 mg, 431 μmol, 1.1 equiv.) as the starting materials, Ephos Pd G4 (36 mg, 39 μmol, 0.1 equiv.) as the catalyst to give tert-butyl 1-oxo-2-(5-(trifluoromethyl)-1,3,4-thiadiazol-2-yl)-2,8-diazaspiro[4.5]decane-8-carboxylate (80 mg, 50%) as a white solid. MS m/z: 407 [M+H]⁺.

Step 2: 2-(5-(trifluoromethyl)-1,3,4-thiadiazol-2-yl)-2,8-diazaspiro[4.5]decan-1-one: Followed the general procedure H using tert-butyl 1-oxo-2-(5-(trifluoromethyl)-1,3,4-thiadiazol-2-yl)-2,8-diazaspiro[4.5]decane-8-carboxylate (80 mg) as the starting material to give the crude product (60 mg). MS m/z: 307 [M+H]⁺.

Step 3: 8-(1-(2,2-difluoroethyl)-1H-pyrazolo[3,4-b]pyrazin-6-yl)-2-(5-(trifluoromethyl)-1,3,4-thiadiazol-2-yl)-2,8-diazaspiro[4.5]decan-1-one: Followed the general procedure I using 2-(5-(trifluoromethyl)-1,3,4-thiadiazol-2-yl)-2,8-diazaspiro[4.5]decan-1-one (60 mg, 195 μmol, 1.0 equiv.) and 6-chloro-1-(2,2-difluoroethyl)-1H-pyrazolo[3,4-b]pyrazine (43 mg, 195 μmol, 1.0 equiv.) as the starting materials to give 8-(1-(2,2-difluoroethyl)-1H-pyrazolo[3,4-b]pyrazin-6-yl)-2-(5-(trifluoromethyl)-1,3,4-thiadiazol-2-yl)-2,8-diazaspiro[4.5]decan-1-one (18 mg, 18%) as a white solid. ¹H NMR (400 MHz, DMSO-d₆) δ 8.52 (s, 1H), 8.15 (s, 1H), 6.58-6.27 (m, 1H), 4.71 (td, J=15.0, 3.8 Hz, 2H), 4.36 (dt, J=13.3, 4.3 Hz, 2H), 4.22 (dd, J=8.6, 5.6 Hz, 2H), 3.46 (ddd, J=13.7, 10.3, 3.4 Hz, 2H), 2.37 (t, J=7.1 Hz, 2H), 2.07 (s, 2H), 1.84 (dtd, J=25.1, 9.7, 5.2 Hz, 4H). MS m/z: 488.95 [M+H]⁺.

### Example 841: 8-(1-(2,2-difluoroethyl)-1H-pyrazolo[3,4-b]pyrazin-6-yl)-2-(2-(trifluoromethyl)thiazol-5-yl)-2,8-diazaspiro[4.5]decan-1-one

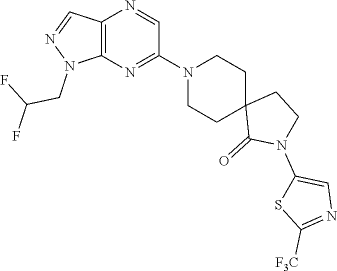 [SMILES: O=C1N(c2cnc(C(F)(F)F)s2)CCC12CCN(c1cnc3cnn(CC(F)F)c3n1)CC2]

Step 1: tert-butyl 1-oxo-2-(2-(trifluoromethyl)thiazol-5-yl)-2,8-diazaspiro[4.5]decane-8-carboxylate: Followed the general procedure Y using tert-butyl 1-oxo-2,8-diazaspiro[4.5]decane-8-carboxylate (120 mg, 0.472 mmol, 1.0 equiv.) and 5-bromo-2-(trifluoromethyl)thiazole (164 mg, 0.708 mmol, 1.5 equiv.) as the starting materials, EPhos (50 mg, 0.094 mmol, 0.2 equiv.), EPhos Pd G4 (44 mg, 0.047 mmol, 0.1 equiv.) as the catalysts to give tert-butyl 1-oxo-2-(2-(trifluoromethyl)thiazol-5-yl)-2,8-diazaspiro[4.5]decane-8-carboxylate (138 mg, 57%) as a white solid. MS m/z: 406 [M+H]⁺.

Step 2: 2-(2-(trifluoromethyl)thiazol-5-yl)-2,8-diazaspiro[4.5]decan-1-one hydrochloride: Followed the general procedure B using tert-butyl 1-oxo-2-(2-(trifluoromethyl)thiazol-5-yl)-2,8-diazaspiro[4.5]decane-8-carboxylate (138 mg) as the starting material to give the crude product 2-(2-(trifluoromethyl)thiazol-5-yl)-2,8-diazaspiro[4.5]decan-1-one hydrochloride (80 mg). MS m/z: 306 [M+H]⁺.

Step 3: 8-(1-(2,2-difluoroethyl)-1H-pyrazolo[3,4-b]pyrazin-6-yl)-2-(2-(trifluoromethyl)thiazol-5-yl)-2,8-diazaspiro[4.5]decan-1-one: Followed the general procedure I using 2-(2-(trifluoromethyl)thiazol-5-yl)-2,8-diazaspiro[4.5]decan-1-one hydrochloride (80 mg, 0.262 mmol, 1.0 equiv.) and 6-chloro-1-(2,2-difluoroethyl)-1H-pyrazolo[3,4-b]pyrazine (57.2 mg, 0.262 mmol, 1.0 equiv.) as the starting materials to give 8-(1-(2,2-difluoroethyl)-1H-pyrazolo[3,4-b]pyrazin-6-yl)-2-(2-(trifluoromethyl)thiazol-5-yl)-2,8-diazaspiro[4.5]decan-1-one (57.7 mg, 45%) as a white solid. ¹H NMR (400 MHz, DMSO-d₆) δ 8.52 (s, 1H), 8.15 (s, 1H), 7.93-7.88 (m, 1H), 6.62-6.23 (m, 1H), 4.71 (td, J=15.0, 3.8 Hz, 2H), 4.43-4.35 (m, 2H), 4.02 (t, J=7.1 Hz, 2H), 3.47-3.35 (m, 2H), 2.35 (d, J=7.1 Hz, 2H), 1.90-1.79 (m, 2H), 1.73 (d, J=13.4 Hz, 2H), 1.24 (s, 1H). MS m/z: 488.1 [M+H]⁺.

### Example 842: 8-(1-(2,2-difluoroethyl)-1H-pyrazolo[3,4-b]pyrazin-6-yl)-2-(5-methyl-2-(trifluoromethyl)pyrimidin-4-yl)-2,8-diazaspiro[4.5]decan-1-one; Example 843: 8-(1-(2,2-difluoroethyl)-1H-pyrazolo[3,4-b]pyrazin-6-yl)-2-((2-(trifluoromethyl)pyrimidin-5-yl)methyl)-2,8-diazaspiro[4.5]decan-1-one

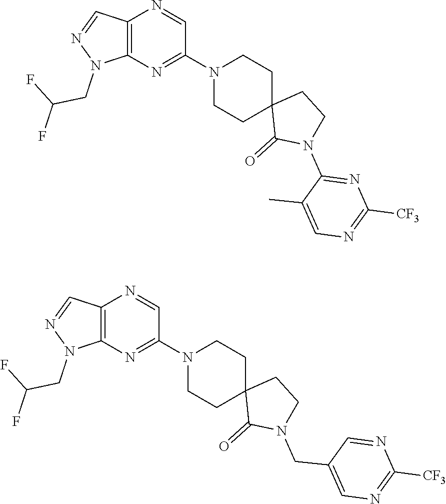 [SMILES: Cc1cnc(C(F)(F)F)nc1N1CCC2(CCN(c3cnc4cnn(CC(F)F)c4n3)CC2)C1=O ; O=C1N(Cc2cnc(C(F)(F)F)nc2)CCC12CCN(c1cnc3cnn(CC(F)F)c3n1)CC2]

Step 1: tert-butyl 2-(5-methyl-2-(trifluoromethyl)pyrimidin-4-yl)-1-oxo-2,8-diazaspiro[4.5]decane-8-carboxylate; tert-butyl 1-oxo-2-((2-(trifluoromethyl)pyrimidin-5-yl)methyl)-2,8-diazaspiro[4.5]decane-8-carboxylate: Followed the general procedure E using tert-butyl 1-oxo-2,8-diazaspiro[4.5]decane-8-carboxylate (500 mg, 1.96 mmol, 1.0 equiv.) and 5-(bromomethyl)-2-(trifluoromethyl)pyrimidine (567 mg, 2.35 mmol, 1.2 equiv.) as the starting materials to give tert-butyl 2-(5-methyl-2-(trifluoromethyl)pyrimidin-4-yl)-1-oxo-2,8-diazaspiro[4.5]decane-8-carboxylate and tert-butyl 1-oxo-2-((2-(trifluoromethyl)pyrimidin-5-yl)methyl)-2,8-diazaspiro[4.5]decane-8-carboxylate (215 mg, 26%) as a white solid. MS m/z: 415 [M+H]⁺.

Step 2: 2-(5-methyl-2-(trifluoromethyl)pyrimidin-4-yl)-2,8-diazaspiro[4.5]decan-1-one hydrochloride: 2-((2-(trifluoromethyl)pyrimidin-5-yl)methyl)-2,8-diazaspiro[4.5]decan-1-one: Followed the general procedure B using tert-butyl 2-(5-methyl-2-(trifluoromethyl)pyrimidin-4-yl)-1-oxo-2,8-diazaspiro[4.5]decane-8-carboxylate and tert-butyl 1-oxo-2-((2-(trifluoromethyl)pyrimidin-5-yl)methyl)-2,8-diazaspiro[4.5]decane-8-carboxylate (215 mg) as the starting material to give the crude product 2-(5-methyl-2-(trifluoromethyl)pyrimidin-4-yl)-2,8-diazaspiro[4.5]decan-1-one hydrochloride and 2-((2-(trifluoromethyl)pyrimidin-5-yl)methyl)-2,8-diazaspiro[4.5]decan-1-one (140 mg). MS m/z: 315 [M+H]⁺.

Step 3: 8-(1-(2,2-difluoroethyl)-1H-pyrazolo[3,4-b]pyrazin-6-yl)-2-(5-methyl-2-(trifluoromethyl)pyrimidin-4-yl)-2,8-diazaspiro[4.5]decan-1-one: 8-(1-(2,2-difluoroethyl)-1H-pyrazolo[3,4-b]pyrazin-6-yl)-2-((2-(trifluoromethyl)pyrimidin-5-yl)methyl)-2,8-diazaspiro[4.5]decan-1-one: Followed the general procedure I using 2-(5-methyl-2-(trifluoromethyl)pyrimidin-4-yl)-2,8-diazaspiro[4.5]decan-1-one hydrochloride and 2-((2-(trifluoromethyl)pyrimidin-5-yl)methyl)-2,8-diazaspiro[4.5]decan-1-one (140 mg, 0.174 mmol, 1.00 equiv.) and 6-chloro-1-(2,2-difluoroethyl)-1H-pyrazolo[3,4-b]pyrazine (42 mg, 0.192 mmol, 1.10 equiv.) as the starting materials to give 8-(1-(2,2-difluoroethyl)-1H-pyrazolo[3,4-b]pyrazin-6-yl)-2-(5-methyl-2-(trifluoromethyl)pyrimidin-4-yl)-2,8-diazaspiro[4.5]decan-1-one (22 mg, 20%) as a white solid and 8-(1-(2,2-difluoroethyl)-1H-pyrazolo[3,4-b]pyrazin-6-yl)-2-((2-(trifluoromethyl)pyrimidin-5-yl)methyl)-2,8-diazaspiro[4.5]decan-1-one (24.8 mg, 22%) as a white solid.

Example 842: ¹H NMR (400 MHz, DMSO-d₆) δ 8.94 (s, 1H), 8.52 (s, 1H), 8.15 (s, 1H), 6.60-6.28 (m, 1H), 4.71 (td, J=15.0, 3.8 Hz, 2H), 4.42 (d, J=13.6 Hz, 2H), 4.04 (t, J=6.9 Hz, 2H), 3.44-3.35 (m, 2H), 2.33-2.28 (m, 2H), 2.23 (s, 3H), 1.88-1.72 (m, 4H). MS m/z: 497.2 [M+H]⁺.

Example 843: ¹H NMR (400 MHz, DMSO-d₆) δ 8.93 (s, 2H), 8.49 (s, 1H), 8.14 (s, 1H), 6.44 (tt, J=54.9, 3.8 Hz, 1H), 4.70 (td, J=15.1, 3.8 Hz, 2H), 4.57 (s, 2H), 4.40 (d, J=13.3 Hz, 2H), 3.38 (t, J=6.8 Hz, 2H), 3.28 (d, J=7.6 Hz, 2H), 2.08 (t, J=6.8 Hz, 2H), 1.81-1.70 (m, 2H), 1.57 (d, J=13.4 Hz, 2H). MS m/z: 497.2 [M+H]⁺.

### Example 844: 8-(1-(2,2-difluoroethyl)-1H-pyrazolo[3,4-b]pyrazin-6-yl)-2-((2-(trifluoromethyl)pyrimidin-4-yl)methyl)-2,8-diazaspiro[4.5]decan-1-one

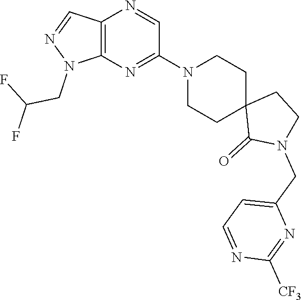 [SMILES: O=C1N(Cc2ccnc(C(F)(F)F)n2)CCC12CCN(c1cnc3cnn(CC(F)F)c3n1)CC2]

Step 1: (2-(trifluoromethyl)pyrimidin-4-yl)methanol: Followed the general procedure X using 4-chloro-2-(trifluoromethyl)pyrimidine (1000 mg, 5.48 mmol, 1.0 equiv.) and (tributylstannyl)methanol (3518 mg, 11.0 mmol, 2.0 equiv.) as the starting materials to give (2-(trifluoromethyl)pyrimidin-4-yl)methanol (600 mg, 61.5%) as a yellow oil. MS m/z: 179 [M+H]⁺.

Step 2: (2-(trifluoromethyl)pyrimidin-4-yl)methyl methanesulfonate: Followed the general procedure R using (2-(trifluoromethyl)pyrimidin-4-yl)methanol (600 mg, 3.35 mmol, 1.0 equiv.) and MsCl (461 mg, 4.02 mmol, 1.2 equiv.) as the starting materials to give (2-(trifluoromethyl)pyrimidin-4-yl)methyl methanesulfonate (250 mg, 29.0%) as a yellow oil. MS m/z: 257 [M+H]⁺.

Step 3: tert-butyl 1-oxo-2-((2-(trifluoromethyl)pyrimidin-4-yl)methyl)-2,8-diazaspiro[4.5]decane-8-carboxylate: Followed the general procedure E using (2-(trifluoromethyl)pyrimidin-4-yl)methyl methanesulfonate (250 mg, 0.973 mmol, 1.0 equiv.) and tert-butyl 1-oxo-2,8-diazaspiro[4.5]decane-8-carboxylate (495 mg, 1.95 mmol, 2.0 equiv.) as the starting materials to give tert-butyl 1-oxo-2-((2-(trifluoromethyl)pyrimidin-4-yl)methyl)-2,8-diazaspiro[4.5]decane-8-carboxylate (70.0 mg, 17.3%) as a white solid. MS m/z: 415 [M+H]⁺.

Step 4: 2-((2-(trifluoromethyl)pyrimidin-4-yl)methyl)-2,8-diazaspiro[4.5]decan-1-one hydrochloride: Followed the general procedure B using tert-butyl 1-oxo-2-((2-(trifluoromethyl)pyrimidin-4-yl)methyl)-2,8-diazaspiro[4.5]decane-8-carboxylate (70.0 mg, 0.168 mmol, 1.0 equiv) as the starting material to give the crude product 2-((2-(trifluoromethyl)pyrimidin-4-yl)methyl)-2,8-diazaspiro[4.5]decan-1-one hydrochloride (45.0 mg). MS m/z: 315 [M+H]⁺.

Step 5: 8-(1-(2,2-difluoroethyl)-1H-pyrazolo[3,4-b]pyrazin-6-yl)-2-((2-(trifluoromethyl)pyrimidin-4-yl)methyl)-2,8-diazaspiro[4.5]decan-1-one: Followed the general procedure I using 2-((2-(trifluoromethyl)pyrimidin-4-yl)methyl)-2,8-diazaspiro[4.5]decan-1-one hydrochloride (45.0 mg, 0.143 mmol, 1.0 equiv.) and 6-chloro-1-(2,2-difluoroethyl)-1H-pyrazolo[3,4-b]pyrazine (31.3 mg, 0.143 mmol, 1.0 equiv.) as the starting materials to give 8-(1-(2,2-difluoroethyl)-1H-pyrazolo[3,4-b]pyrazin-6-yl)-2-((2-(trifluoromethyl)pyrimidin-4-yl)methyl)-2,8-diazaspiro[4.5]decan-1-one (12.8 mg, 19.7%) as a white solid. ¹H NMR (400 MHz, DMSO-d₆) δ 9.01 (d, 1H), 8.50 (s, 1H), 8.13 (s, 1H), 7.68 (d, 1H), 6.44 (tt, 1H), 4.91-4.60 (m, 4H), 4.43-4.34 (m, 2H), 3.45 (t, 2H), 3.41-3.33 (m, 2H), 2.13 (t, 2H), 1.85-1.74 (m, 2H), 1.62 (dt, 3.5 Hz, 2H). MS m/z: 497.15 [M+H]⁺.

### Example 845: 8-(1-(2,2-difluoroethyl)-1H-pyrazolo[3,4-b]pyrazin-6-yl)-2-(1-methyl-6-oxo-1,6-dihydropyridin-3-yl)-2,8-diazaspiro[4.5]decan-1-one

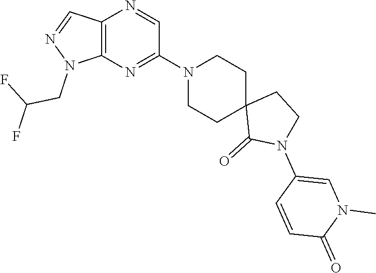 [SMILES: Cn1cc(N2CCC3(CCN(c4cnc5cnn(CC(F)F)c5n4)CC3)C2=O)ccc1=O]

Step 1: tert-butyl 2-(1-methyl-6-oxo-1,6-dihydropyridin-3-yl)-1-oxo-2,8-diazaspiro[4.5]decane-8-carboxylate: Followed the general procedure Y using tert-butyl 1-oxo-2,8-diazaspiro[4.5]decane-8-carboxylate (100 mg, 392 μmol, 1.0 equiv.) and 5-bromo-1-methylpyridin-2(1H)-one (81 mg, 431 μmol, 1.0 equiv.) as the starting materials, Ephos Pd G4 (36 mg, 39 μmol, 0.1 equiv.) as the catalyst to give tert-butyl 2-(1-methyl-6-oxo-1,6-dihydropyridin-3-yl)-1-oxo-2,8-diazaspiro[4.5]decane-8-carboxylate (100 mg, 70%) as a white solid. MS m/z: 362 [M+H]⁺.

Step 2: 2-(1-methyl-6-oxo-1,6-dihydropyridin-3-yl)-2,8-diazaspiro[4.5]decan-1-one: Followed the general procedure H using tert-butyl 2-(1-methyl-6-oxo-1,6-dihydropyridin-3-yl)-1-oxo-2,8-diazaspiro[4.5]decane-8-carboxylate (100 mg) as the starting material to give the crude product 2-(1-methyl-6-oxo-1,6-dihydropyridin-3-yl)-2,8-diazaspiro[4.5]decan-1-one (80 mg). MS m/z: 262 [M+H]⁺.

Step 3: 8-(1-(2,2-difluoroethyl)-1H-pyrazolo[3,4-b]pyrazin-6-yl)-2-(1-methyl-6-oxo-1,6-dihydropyridin-3-yl)-2,8-diazaspiro[4.5]decan-1-one: Followed the general procedure I using 2-(1-methyl-6-oxo-1,6-dihydropyridin-3-yl)-2,8-diazaspiro[4.5]decan-1-one (80 mg, 305 μmol, 1.0 equiv.) and 6-chloro-1-(2,2-difluoroethyl)-1H-pyrazolo[3,4-b]pyrazine (66 mg, 305 μmol, 1.0 equiv.) as the starting materials to give 8-(1-(2,2-difluoroethyl)-1H-pyrazolo[3,4-b]pyrazin-6-yl)-2-(1-methyl-6-oxo-1,6-dihydropyridin-3-yl)-2,8-diazaspiro[4.5]decan-1-one (39 mg, 28%) as a white solid. ¹H NMR (400 MHz, DMSO-d₆) δ 8.49 (d, J=10.9 Hz, 1H), 8.13 (d, J=4.4 Hz, 1H), 7.95 (d, J=2.9 Hz, 1H), 7.78 (dd, J=9.7, 3.0 Hz, 1H), 6.60-6.28 (m, 2H), 4.70 (tt, J=15.1, 3.9 Hz, 2H), 4.41 (dt, J=13.9, 4.2 Hz, 2H), 3.71 (t, J=6.9 Hz, 2H), 3.44 (s, 3H), 3.38-3.32 (m, 2H), 2.16 (t, J=6.9 Hz, 2H), 1.85-1.69 (m, 2H), 1.69-1.59 (m, 2H). MS m/z: 444.25 [M+H]⁺.

### Example 846: 8-(1-(2,2-difluoroethyl)-1H-pyrazolo[3,4-b]pyrazin-6-yl)-2-(1-ethyl-6-oxo-1,6-dihydropyridin-3-yl)-2,8-diazaspiro[4.5]decan-1-one

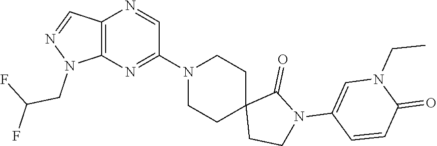 [SMILES: CCn1cc(N2CCC3(CCN(c4cnc5cnn(CC(F)F)c5n4)CC3)C2=O)ccc1=O]

Step 1: 5-bromo-1-ethylpyridin-2(1H)-one: Followed the general procedure I using 5-bromopyridin-2(1H)-one (150 mg, 0.862 mmol, 1.0 equiv.) and iodoethane (161 mg, 1.04 mmol, 1.2 equiv.) as the starting materials to give 5-bromo-1-ethylpyridin-2(1H)-one (110 mg, 63%) as a white solid. MS m/z: 202 [M+H]⁺.

Step 2: tert-butyl 2-(1-ethyl-6-oxo-1,6-dihydropyridin-3-yl)-1-oxo-2,8-diazaspiro[4.5]decane-8-carboxylate: Followed the general procedure Y using 5-bromo-1-ethylpyridin-2(1H)-one (100 mg, 0.495 mmol, 1.0 equiv.) and tert-butyl 1-oxo-2,8-diazaspiro[4.5]decane-8-carboxylate (139 mg, 0.545 mmol, 1.1 equiv.) as the starting materials, Ephos Pd G3 (45.5 mg, 0.054 mmol, 0.1 equiv.) as the catalyst to give tert-butyl 2-(1-ethyl-6-oxo-1,6-dihydropyridin-3-yl)-1-oxo-2,8-diazaspiro[4.5]decane-8-carboxylate (90 mg, 48%) as a white solid. MS m/z: 376 [M+H]⁺.

Step 3: 2-(1-ethyl-6-oxo-1,6-dihydropyridin-3-yl)-2,8-diazaspiro[4.5]decan-1-one hydrochloride: Followed the general procedure B using tert-butyl 2-(1-ethyl-6-oxo-1,6-dihydropyridin-3-yl)-1-oxo-2,8-diazaspiro[4.5]decane-8-carboxylate (90 mg) as the starting material to give the crude product 2-(1-ethyl-6-oxo-1,6-dihydropyridin-3-yl)-2,8-diazaspiro[4.5]decan-1-one hydrochloride (60 mg). MS m/z: 276 [M+H]⁺.

Step 4: 8-(1-(2,2-difluoroethyl)-1H-pyrazolo[3,4-b]pyrazin-6-1-2-(1-ethyl-6-oxo-1,6-dihydropyridin-3-yl)-2,8-diazaspiro[4.5]decan-1-one: Followed the general procedure I using 2-(1-ethyl-6-oxo-1,6-dihydropyridin-3-yl)-2,8-diazaspiro[4.5]decan-1-one hydrochloride (60 mg, 0.217 mmol, 1.0 equiv.) and 6-chloro-1-(2,2-difluoroethyl)-1H-pyrazolo[3,4-b]pyrazine (52.4 mg, 0.239 mmol, 1.1 equiv.) as the starting materials to give 8-(1-(2,2-difluoroethyl)-1H-pyrazolo[3,4-b]pyrazin-6-yl)-2-(1-ethyl-6-oxo-1,6-dihydropyridin-3-yl)-2,8-diazaspiro[4.5]decan-1-one (64.4 mg, 64%) as an off-white solid. ¹H NMR (400 MHz, DMSO-d₆) δ 8.50 (s, 1H), 8.15 (d, J=5.8 Hz, 1H), 7.96 (d, J=2.9 Hz, 1H), 7.78 (dd, J=9.7, 3.0 Hz, 1H), 6.63-6.28 (m, 2H), 4.77-4.64 (m, 2H), 4.47-4.37 (m, 2H), 3.96-3.87 (m, 2H), 3.73 (t, J=6.8 Hz, 2H), 3.34 (d, J=3.0 Hz, 2H), 2.17 (t, J=6.9 Hz, 2H), 1.85-1.74 (m, 2H), 1.68-1.58 (m, 2H), 1.22 (t, J=7.1 Hz, 3H). MS m/z: 458.1 [M+H]⁺.

### Example 847: 8-(1-(2,2-difluoroethyl)-1H-pyrazolo[3,4-b]pyrazin-6-yl)-2-(1-ethyl-6-oxo-1,6-dihydropyridazin-3-yl)-2,8-diazaspiro[4.5]decan-1-one

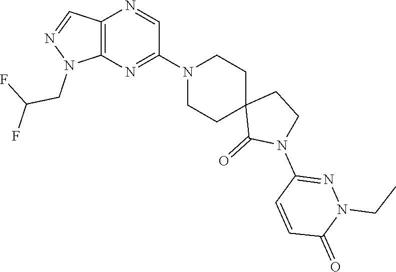 [SMILES: CCn1nc(N2CCC3(CCN(c4cnc5cnn(CC(F)F)c5n4)CC3)C2=O)ccc1=O]

Step 1: 6-chloro-2-ethylpyridazin-3(2H)-one: Followed the general procedure K using 6-chloropyridazin-3(2H)-one (500 mg, 3.81 mmol, 1.0 equiv.) and Iodoethane (655 mg, 4.19 mmol, 1.1 equiv.) as the starting materials to give 6-chloro-2-ethylpyridazin-3(2H)-one (450 mg, 74%) as a white solid. MS m/z: 159 [M+H]⁺.

Step 2: tert-butyl 2-(1-ethyl-6-oxo-1,6-dihydropyridazin-3-yl)-1-oxo-2,8-diazaspiro[4.5]decane-8-carboxylate: Followed the general procedure I using 6-chloro-2-ethylpyridazin-3(2H)-one (450 mg, 2.83 mmol, 1.0 equiv.) and tert-butyl 1-oxo-2,8-diazaspiro[4.5]decane-8-carboxylate (794 mg, 3.11 mmol, 1.1 equiv.) as the starting materials to give tert-butyl 2-(1-ethyl-6-oxo-1,6-dihydropyridazin-3-yl)-1-oxo-2,8-diazaspiro[4.5]decane-8-carboxylate (120 mg, 11%) as a white solid. MS m/z: 377 [M+H]⁺.

Step 3: 2-(1-ethyl-6-oxo-1,6-dihydropyridazin-3-yl)-2,8-diazaspiro[4.5]decan-1-one: Followed the general procedure H using tert-butyl 2-(1-ethyl-6-oxo-1,6-dihydropyridazin-3-yl)-1-oxo-2,8-diazaspiro[4.5]decane-8-carboxylate (120 mg) as the starting material to give the crude product 2-(1-ethyl-6-oxo-1,6-dihydropyridazin-3-yl)-2,8-diazaspiro[4.5]decan-1-one (90 mg). MS m/z: 277 [M+H]⁺.

Step 4: 8-(1-(2,2-difluoroethyl)-1H-pyrazolo[3,4-b]pyrazin-6-yl)-2-(1-ethyl-6-oxo-1,6-dihydropyridazin-3-yl)-2,8-diazaspiro[4.5]decan-1-one: Followed the general procedure I using 2-(1-ethyl-6-oxo-1,6-dihydropyridazin-3-yl)-2,8-diazaspiro[4.5]decan-1-one (90 mg, 324 μmol, 1.0 equiv.) and 6-chloro-1-(2,2-difluoroethyl)-1H-pyrazolo[3,4-b]pyrazine (79 mg, 357 μmol, 1.1 equiv.) as the starting materials to give 8-(1-(2,2-difluoroethyl)-1H-pyrazolo[3,4-b]pyrazin-6-yl)-2-(1-ethyl-6-oxo-1,6-dihydropyridazin-3-yl)-2,8-diazaspiro[4.5]decan-1-one (13 mg, 9%) as a white solid. ¹H NMR (400 MHz, DMSO-d₆) δ 8.51 (s, 1H), 8.35 (d, J=10.0 Hz, 1H), 8.15 (s, 1H), 7.00 (d, J=10.1 Hz, 1H), 6.57-6.32 (m, 1H), 4.70 (td, J=15.1, 3.9 Hz, 2H), 4.43 (d, J=13.7 Hz, 2H), 4.03 (q, J=7.2 Hz, 2H), 3.85 (t, J=7.0 Hz, 2H), 3.31 (s, 2H), 2.18 (t, J=7.0 Hz, 2H), 1.86-1.74 (m, 2H), 1.68 (d, J=13.4 Hz, 2H), 1.27 (d, J=7.1 Hz, 3H), 1.24 (d, J=3.7 Hz, 3H), 0.85 (t, J=6.5 Hz, 1H). MS m/z: 459.10 [M+H]⁺.

### Example 848: 1-(2,4-difluorobenzyl)-8-(1-(2,2-difluoroethyl)-1H-pyrazolo[3,4-b]pyrazin-6-yl)-3-(4-(trifluoromethyl)pyridin-2-yl)-1,3,8-triazaspiro[4.5]decane-2,4-dione

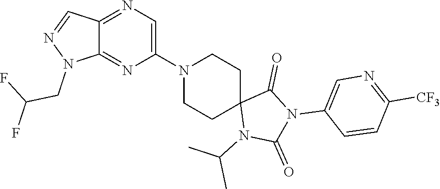 [SMILES: CC(C)N1C(=O)N(c2ccc(C(F)(F)F)nc2)C(=O)C12CCN(c1cnc3cnn(CC(F)F)c3n1)CC2]

Step 1: tert-butyl 1-isopropyl-2,4-dioxo-3-(6-(trifluoromethyl)pyridin-3-yl)-1,3,8-triazaspiro[4.5]decane-8-carboxylate: Followed the general procedure K using tert-butyl 2,4-dioxo-3-(6-(trifluoromethyl)pyridin-3-yl)-1,3,8-triazaspiro[4.5]decane-8-carboxylate (200 mg, 483 μmol, 1.0 equiv.) and 2-iodopropane (82.1 mg, 725 μmol, 1.5 equiv.) as the starting materials to give tert-butyl 1-isopropyl-2,4-dioxo-3-(6-(trifluoromethyl)pyridin-3-yl)-1,3,8-triazaspiro[4.5]decane-8-carboxylate (55 mg, 25%) as a yellow oil. MS m/z: 457 [M+H]⁺.

Step 2: 1-isopropyl-3-(6-(trifluoromethyl)pyridin-3-yl)-1,3,8-triazaspiro[4.5]decane-2,4-dione hydrochloride: Followed the general procedure B using tert-butyl 1-isopropyl-2,4-dioxo-3-(6-(trifluoromethyl)pyridin-3-yl)-1,3,8-triazaspiro[4.5]decane-8-carboxylate (55 mg) as the starting material to give the crude product 1-isopropyl-3-(6-(trifluoromethyl)pyridin-3-yl)-1,3,8-triazaspiro[4.5]decane-2,4-dione hydrochloride (45 mg) as a yellow solid. MS m/z: 357 [M+H]+.

Step 3: 8-(1-(2,2-difluoroethyl)-1H-pyrazolo[3,4-b]pyrazin-6-yl)-1-isopropyl-3-(6-(trifluoromethyl)pyridin-3-yl)-1,3,8-triazaspiro[4.5]decane-2,4-dione: Followed the general procedure I using 1-isopropyl-3-(6-(trifluoromethyl)pyridin-3-yl)-1,3,8-triazaspiro[4.5]decane-2,4-dione (40 mg, 112 μmol, 1.0 equiv.) and 6-chloro-1-(2,2-difluoroethyl)-1H-pyrazolo[3,4-b]pyrazine (29.4 mg, 135 μmol, 1.2 equiv.) as the starting materials to give 8-(1-(2,2-difluoroethyl)-1H-pyrazolo[3,4-b]pyrazin-6-yl)-1-isopropyl-3-(6-(trifluoromethyl)pyridin-3-yl)-1,3,8-triazaspiro[4.5]decane-2,4-dione (25 mg, 41%) as a white solid. ¹H NMR (400 MHz, DMSO-d₆) δ 8.91 (d, J=2.3 Hz, 1H), 8.54 (s, 1H), 8.25 (dd, J=8.5, 2.3 Hz, 1H), 8.16 (s, 1H), 8.11 (d, J=8.4 Hz, 1H), 6.45 (tt, J=54.9, 3.8 Hz, 1H), 4.79-4.61 (m, 4H), 3.68-3.55 (m, 3H), 2.17 (d, J=13.1 Hz, 2H), 2.12-2.03 (m, 2H), 1.35 (d, J=6.7 Hz, 6H). MS m/z: 539.05 [M+H]⁺.

### Example 849: 8-(1-(2,2-difluoroethyl)-1H-pyrazolo[3,4-b]pyrazin-6-yl)-1-ethyl-3-(6-(trifluoromethyl)pyridin-3-yl)-1,3,8-triazaspiro[4.5]decane-2,4-dione

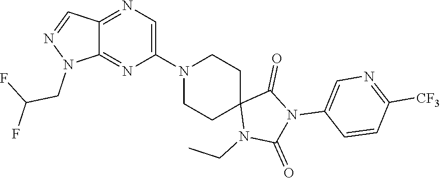 [SMILES: CCN1C(=O)N(c2ccc(C(F)(F)F)nc2)C(=O)C12CCN(c1cnc3cnn(CC(F)F)c3n1)CC2]

Step 1: tert-butyl 1-ethyl-2,4-dioxo-3-(6-(trifluoromethyl)pyridin-3-yl)-1,3,8-triazaspiro[4.5]decane-8-carboxylate: Followed the general procedure K using tert-butyl 2,4-dioxo-3-(6-(trifluoromethyl)pyridin-3-yl)-1,3,8-triazaspiro[4.5]decane-8-carboxylate (70 mg, 169 μmol, 1.0 equiv.) and iodoethane (39.6 mg, 254 μmol, 1.5 equiv.) as the starting materials to give tert-butyl 1-ethyl-2,4-dioxo-3-(6-(trifluoromethyl)pyridin-3-yl)-1,3,8-triazaspiro[4.5]decane-8-carboxylate (65 mg, 87%) as a yellow oil. MS m/z: 443 [M+H]⁺.

Step 2: 1-ethyl-3-(6-(trifluoromethyl)pyridin-3-yl)-1,3,8-triazaspiro[4.5]decane-2,4-dione hydrochloride: Followed the general procedure B using tert-butyl 1-ethyl-2,4-dioxo-3-(6-(trifluoromethyl)pyridin-3-yl)-1,3,8-triazaspiro[4.5]decane-8-carboxylate (65 mg) as the starting material to give the crude product 1-ethyl-3-(6-(trifluoromethyl)pyridin-3-yl)-1,3,8-triazaspiro[4.5]decane-2,4-dione hydrochloride (50 mg) as a yellow solid. MS m/z: 343 [M+H]+.

Step 3: 8-(1-(2,2-difluoroethyl)-1H-pyrazolo[3,4-b]pyrazin-6-yl)-1-ethyl-3-(6-(trifluoromethyl)pyridin-3-yl)-1,3,8-triazaspiro[4.5]decane-2,4-dione: Followed the general procedure I using 1-ethyl-3-(6-(trifluoromethyl)pyridin-3-yl)-1,3,8-triazaspiro[4.5]decane-2,4-dione hydrochloride (50 mg, 158 μmol, 1.0 equiv.) and 6-chloro-1-(2,2-difluoroethyl)-1H-pyrazolo[3,4-b]pyrazine (38.1 mg, 175 μmol, 1.2 equiv.) as the starting materials to give 8-(1-(2,2-difluoroethyl)-1H-pyrazolo[3,4-b]pyrazin-6-yl)-1-ethyl-3-(6-(trifluoromethyl)pyridin-3-yl)-1,3,8-triazaspiro[4.5]decane-2,4-dione (14.5 mg, 19%) as a white solid. ¹H NMR (400 MHz, DMSO-d₆) δ 8.93 (d, J=2.3 Hz, 1H), 8.54 (s, 1H), 8.26 (dd, J=8.5, 2.3 Hz, 1H), 8.16 (s, 1H), 8.12 (d, J=8.4 Hz, 1H), 6.44 (tt, J=55.0, 3.9 Hz, 1H), 4.77-4.60 (m, 4H), 3.69-3.57 (m, 2H), 3.37-3.34 (m, 1H), 3.29 (s, 1H), 2.20-2.07 (m, 4H), 1.16 (t, J=7.0 Hz, 3H). MS m/z: 525.00 [M+H]⁺.

### Example 850: 8-(1-(2,2-difluoroethyl)-1H-pyrazolo[3,4-b]pyrazin-6-yl)-1-ethyl-3-(2-(trifluoromethyl)pyridin-4-yl)-1,3,8-triazaspiro[4.5]decane-2,4-dione

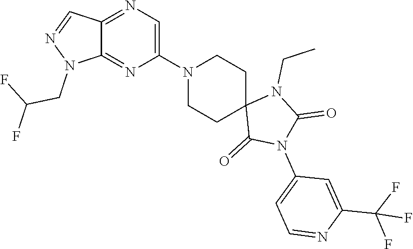 [SMILES: CCN1C(=O)N(c2ccnc(C(F)(F)F)c2)C(=O)C12CCN(c1cnc3cnn(CC(F)F)c3n1)CC2]

Step 1: tert-butyl 1-ethyl-2,4-dioxo-3-(2-(trifluoromethyl)pyridin-4-yl)-1,3,8-triazaspiro[4.5]decane-8-carboxylate: Followed the general procedure K using tert-butyl 2,4-dioxo-3-[2-(trifluoromethyl)pyridin-4-yl]-1,8-diazaspiro[4.5]decane-8-carboxylate (80 mg, 0.194 mmol, 1 equiv) and iodoethane (36.22 mg, 0.233 mmol, 1.2 equiv) as the starting materials to give tert-butyl 1-ethyl-2,4-dioxo-3-[2-(trifluoromethyl)pyridin-4-yl]-1,3,8-triazaspiro[4.5]decane-8-carboxylate (70 mg, 81%) as a white solid. MS m/z: 443 [M+H]⁺.

Step 2: 1-ethyl-3-(2-(trifluoromethyl)pyridin-4-yl)-1,3,8-triazaspiro[4.5]decane-2,4-dione hydrochloride: Followed the general procedure B using tert-butyl 1-ethyl-2,4-dioxo-3-[2-(trifluoromethyl)pyridin-4-yl]-1,3,8-triazaspiro[4.5]decane-8-carboxylate (70 mg, 0.158 mmol, 1 equiv) as the starting material to give the crude product 1-ethyl-3-(2-(trifluoromethyl)pyridin-4-yl)-1,3,8-triazaspiro[4.5]decane-2,4-dione hydrochloride (55 mg). MS m/z: 343 [M+H]⁺.

Step 3: 8-(1-(2,2-difluoroethyl)-1H-pyrazolo[3,4-b]pyrazin-6-yl)-1-ethyl-3-(2-(trifluoromethyl)pyridin-4-yl)-1,3,8-triazaspiro[4.5]decane-2,4-dione: Followed the general procedure I using 1-ethyl-3-[2-(trifluoromethyl)pyridin-4-yl]-1,3,8-triazaspiro[4.5]decane-2,4-dione hydrochloride (54 mg, 0.158 mmol, 1 equiv) and 6-chloro-1-(2,2-difluoroethyl)-1H-pyrazolo[3,4-b]pyrazine (41.38 mg, 0.190 mmol, 1.2 equiv) as the starting materials to give 8-(1-(2,2-difluoroethyl)-1H-pyrazolo[3,4-b]pyrazin-6-yl)-1-ethyl-3-(2-(trifluoromethyl)pyridin-4-yl)-1,3,8-triazaspiro[4.5]decane-2,4-dione (23 mg, 27%) as a white solid. ¹H NMR (400 MHz, DMSO-d₆) δ 8.91 (d, J=5.4 Hz, 1H), 8.54 (s, 1H), 8.21-8.14 (m, 2H), 7.99 (dd, J=5.3, 1.9 Hz, 1H), 6.45 (tt, J=54.9, 3.8 Hz, 1H), 4.80-4.52 (m, 4H), 3.68-3.57 (m, 2H), 3.37-3.34 (m, 1H), 3.32-3.29 (m, 1H), 2.20-2.03 (m, 4H), 1.15 (t, J=7.0 Hz, 3H). MS m/z: 525.25 [M+H]⁺.

### Example 851: 8-(1-(2,2-difluoroethyl)-1H-pyrazolo[3,4-b]pyrazin-6-yl)-1-ethyl-2-(5-(trifluoromethyl)pyridin-2-yl)-2,8-diazaspiro[4.5]decan-3-one

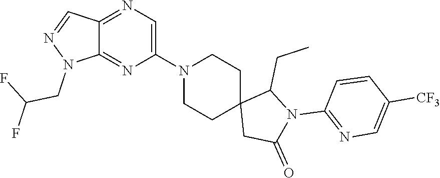 [SMILES: CCC1N(c2ccc(C(F)(F)F)cn2)C(=O)CC12CCN(c1cnc3cnn(CC(F)F)c3n1)CC2]

Step 1: tert-butyl 1-ethyl-3-oxo-2-(5-(trifluoromethyl)pyridin-2-yl)-2,8-diazaspiro[4.5]decane-8-carboxylate: Followed the general procedure E using tert-butyl 1-ethyl-3-oxo-2,8-diazaspiro[4.5]decane-8-carboxylate (100 mg, 0.354 mmol, 1 equiv) and 2-fluoro-5-(trifluoromethyl)pyridine (70.16 mg, 0.425 mmol, 1.2 equiv) as the starting materials to give tert-butyl 1-ethyl-3-oxo-2-(5-(trifluoromethyl)pyridin-2-yl)-2,8-diazaspiro[4.5]decane-8-carboxylate (110 mg, 72%) as a white solid. MS m/z: 428 [M+H]⁺.

Step 2: 1-ethyl-2-(5-(trifluoromethyl)pyridin-2-yl)-2,8-diazaspiro[4.5]decan-3-one hydrochloride: Followed the general procedure B using tert-butyl 1-ethyl-3-oxo-2-(5-(trifluoromethyl)pyridin-2-yl)-2,8-diazaspiro[4.5]decane-8-carboxylate (110 mg, 0.257 mmol, 1 equiv) as the starting material to give the crude product 1-ethyl-2-(5-(trifluoromethyl)pyridin-2-yl)-2,8-diazaspiro[4.5]decan-3-one hydrochloride (40 mg). MS m/z: [M+H]⁺.

Step 3: 8-(1-(2,2-difluoroethyl)-1H-pyrazolo[3,4-b]pyrazin-6-yl)-1-ethyl-2-(5-(trifluoromethyl)pyridin-2-yl)-2,8-diazaspiro[4.5]decan-3-one: Followed the general procedure I using 1-ethyl-2-[5-(trifluoromethyl)pyridin-2-yl]-2,8-diazaspiro[4.5]decan-3-one hydrochloride (40 mg, 0.122 mmol, 1 equiv) and 6-chloro-1-(2,2-difluoroethyl)-1H-pyrazolo[3,4-b]pyrazine (32.05 mg, 0.146 mmol, 1.2 equiv) as the starting materials to give 8-(1-(2,2-difluoroethyl)-1H-pyrazolo[3,4-b]pyrazin-6-yl)-1-ethyl-2-(5-(trifluoromethyl)pyridin-2-yl)-2,8-diazaspiro[4.5]decan-3-one (30 mg, 48%) as a white solid. ¹H NMR (400 MHz, DMSO-d₆) δ 8.80-8.74 (m, 1H), 8.53-8.44 (m, 2H), 8.22 (dd, J=9.0, 2.6 Hz, 1H), 8.13 (s, 1H), 6.44 (tt, J=54.9, 3.8 Hz, 1H), 4.74-4.59 (m, 3H), 4.28-4.14 (m, 2H), 3.59-3.47 (m, 1H), 3.40-3.32 (m, 1H), 2.88-2.66 (m, 2H), 2.01-1.60 (m, 6H), 0.78 (t, J=7.4 Hz, 3H). MS m/z: 510.25 [M+H]⁺.

### Example 852: 8-(1-(2,2-difluoroethyl)-1H-pyrazolo[3,4-b]pyrazin-6-yl)-1-ethyl-2-(6-(trifluoromethyl)pyridin-3-yl)-2,8-diazaspiro[4.5]decan-3-one

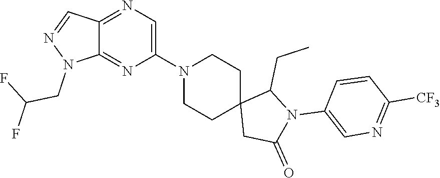 [SMILES: CCC1N(c2ccc(C(F)(F)F)nc2)C(=O)CC12CCN(c1cnc3cnn(CC(F)F)c3n1)CC2]

Step 1: tert-butyl 1-ethyl-3-oxo-2-(6-(trifluoromethyl)pyridin-3-yl)-2,8-diazaspiro[4.5]decane-8-carboxylate: Followed the general procedure Y using tert-butyl 1-ethyl-3-oxo-2,8-diazaspiro[4.5]decane-8-carboxylate (100 mg, 0.354 mmol, 1.0 equiv.) and 5-bromo-2-(trifluoromethyl)pyridine (88 mg, 0.53 mmol, 1.5 equiv.) as the starting materials to give tert-butyl 1-ethyl-3-oxo-2-(6-(trifluoromethyl)pyridin-3-yl)-2,8-diazaspiro[4.5]decane-8-carboxylate (65 mg, 43%) as a colorless oil. MS m/z: 428 [M+H]⁺.

Step 2: 1-ethyl-2-(6-(trifluoromethyl)pyridin-3-yl)-2,8-diazaspiro[4.5]decan-3-one hydrochloride: Followed the general procedure B using tert-butyl 1-ethyl-3-oxo-2-(6-(trifluoromethyl)pyridin-3-yl)-2,8-diazaspiro[4.5]decane-8-carboxylate (65 mg) as the starting material to give the crude product 1-ethyl-2-(6-(trifluoromethyl)pyridin-3-yl)-2,8-diazaspiro[4.5]decan-3-one hydrochloride (40 mg). MS m/z: 328 [M+H]⁺.

Step 3: 8-(1-(2,2-difluoroethyl)-1H-pyrazolo[3,4-b]pyrazin-6-yl)-1-ethyl-2-(6-(trifluoromethyl)pyridin-3-yl)-2,8-diazaspiro[4.5]decan-3-one: Followed the general procedure I using 1-ethyl-2-(6-(trifluoromethyl)pyridin-3-yl)-2,8-diazaspiro[4.5]decan-3-one hydrochloride (40 mg, 0.106 mmol, 1.0 equiv.) and 6-chloro-1-(2,2-difluoroethyl)-1H-pyrazolo[3,4-b]pyrazine (26 mg, 0.117 mmol, 1.1 equiv.) as the starting materials to give 8-(1-(2,2-difluoroethyl)-1H-pyrazolo[3,4-b]pyrazin-6-yl)-1-ethyl-2-(6-(trifluoromethyl)pyridin-3-yl)-2,8-diazaspiro[4.5]decan-3-one (17.6 mg, 32%) as a white solid. ¹H NMR (400 MHz, DMSO-d₆) δ 9.06 (d, J=2.5 Hz, 1H), 8.51 (s, 1H), 8.37 (dd, J=8.6, 2.5 Hz, 1H), 8.14 (s, 1H), 7.94 (d, J=8.7 Hz, 1H), 6.44 (tt, J=54.9, 3.8 Hz, 1H), 4.70 (td, J=15.0, 3.8 Hz, 2H), 4.40-4.19 (m, 3H), 3.63-3.52 (m, 1H), 3.45-3.35 (m, 1H), 2.76 (d, J=17.2 Hz, 1H), 2.61 (d, J=17.1 Hz, 1H), 1.84-1.67 (m, 6H), 0.76 (t, J=7.4 Hz, 3H). MS m/z: 510.15 [M+H]⁺.

### Example 853: 8-[1-(2,2-difluoroethyl)pyrazolo[3,4-b]pyrazin-6-yl]-1-ethyl-2-[2-(trifluoromethyl)pyridin-4-yl]-2,8-diazaspiro[4.5]decan-3-one

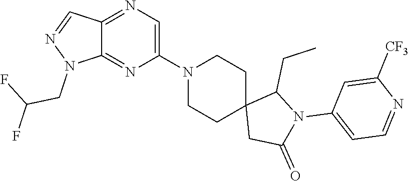 [SMILES: CCC1N(c2ccnc(C(F)(F)F)c2)C(=O)CC12CCN(c1cnc3cnn(CC(F)F)c3n1)CC2]

Step 1: tert-butyl 1-ethyl-3-oxo-2-[2-(trifluoromethyl)pyridin-4-yl]-2,8-diazaspiro[4.5]decane-8 carboxylate: Followed the general procedure Y using tert-butyl 1-ethyl-3-oxo-2,8-diazaspiro[4.5]decane-8-carboxylate (100 mg, 0.354 mmol, 1.0 equiv.) and 4-bromo-2-(trifluoromethyl)pyridine (88 mg, 0.389 mmol, 1.1 equiv.) as the starting materials EPhos Pd G4 (32.5 mg, 0.035 mmol, 0.1 equiv.) as the catalyst to give tert-butyl 1-ethyl-3-oxo-2-[2-(trifluoromethyl)pyridin-4-yl]-2,8-diazaspiro[4.5]decane-8-carboxylate (80 mg, 52%) as a white solid. MS m/z: 428 [M+H]⁺.

Step 2: 1-ethyl-2-[2-(trifluoromethyl)pyridin-4-yl]-2,8-diazaspiro[4.5]decan-3-one hydrochloride: Followed the general procedure B using tert-butyl 1-ethyl-3-oxo-2-[2-(trifluoromethyl)pyridin-4-yl]-2,8-diazaspiro[4.5]decane-8-carboxylate (80 mg) as the starting material to give the crude product 1-ethyl-2-[2-(trifluoromethyl)pyridin-4-yl]-2,8-diazaspiro[4.5]decan-3-one hydrochloride (60 mg). MS m/z: 328 [M+H]⁺.

Step 3: 8-[1-(2,2-difluoroethyl)pyrazolo[3,4-b]pyrazin-6-yl]-1-ethyl-2-[2-(trifluoromethyl)pyridin-4-yl]-2,8-diazaspiro[4.5]decan-3-one: Followed the general procedure I using 1-ethyl-2-[2-(trifluoromethyl)pyridin-4-yl]-2,8-diazaspiro[4.5]decan-3-one hydrochloride (50 mg, 0.137 mmol, 1.0 equiv.) and 6-chloro-1-(2,2-difluoroethyl)pyrazolo[3,4-b]pyrazine (30 mg, 0.137 mmol, 1.0 equiv.) as the starting materials to give 8-[1-(2,2-difluoroethyl)pyrazolo[3,4-b]pyrazin-6-yl]-1-ethyl-2-[2-(trifluoromethyl)pyridin-4-yl]-2,8-diazaspiro[4.5]decan-3-one (20 mg, 28%) as a white solid. ¹H NMR (300 MHz, DMSO-d₆) δ 8.70 (d, 1H), 8.51 (s, 1H), 8.38 (d, 1H), 8.15 (s, 1H), 7.90 (d, 1H), 6.45 (tt, 1H), 4.70 (td, 2H), 4.44-4.40 (m, 1H), 4.25-4.04 (m, 2H), 3.62-3.38 (m, 2H), 2.90-2.60 (m, 2H), 1.92-1.75 (m, 3H), 1.75-1.48 (m, 3H), 0.79 (t, 3H). MS m/z: 510.0 [M+H]⁺.

### Example 854: (R)-8-(1-(2,2-difluoroethyl)-1H-pyrazolo[3,4-b]pyrazin-6-yl)-1-ethyl-2-(2-(trifluoromethyl)pyridin-4-yl)-2,8-diazaspiro[4.5]decan-3-one; Example 855: (S)-8-(1-(2,2-difluoroethyl)-1H-pyrazolo[3,4-b]pyrazin-6-yl)-1-ethyl-2-(2-(trifluoromethyl)pyridin-4-yl)-2,8-diazaspiro[4.5]decan-3-one

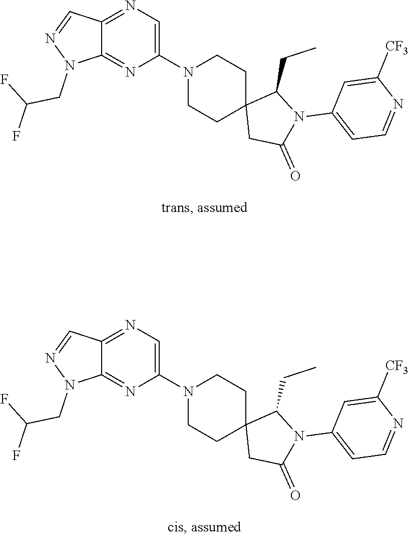 [SMILES: CC[C@H]1N(c2ccnc(C(F)(F)F)c2)C(=O)CC12CCN(c1cnc3cnn(CC(F)F)c3n1)CC2 ; CC[C@@H]1N(c2ccnc(C(F)(F)F)c2)C(=O)CC12CCN(c1cnc3cnn(CC(F)F)c3n1)CC2]

Step 1: 8-(1-(2,2-difluoroethyl)-1H-pyrazolo[3,4-b]pyrazin-6-yl)-1-ethyl-2-(2-(trifluoromethyl)pyridin-4-yl)-2,8-diazaspiro[4.5]decan-3-one: Followed the general procedure Y 1-ethyl-2-(2-(trifluoromethyl)pyridin-4-yl)-2,8-diazaspiro[4.5]decan-3-one (130 mg, 396 μmol, 1.0 equiv.) and 6-chloro-1-(2,2-difluoroethyl)-1H-pyrazolo[3,4-b]pyrazine (86.4 mg, 396 μmol, 1.0 equiv.) as the starting materials, EPhos Pd G4 (54.8 mg, 39.6 μmol, 0.1 equiv.) as the catalyst to give the crude product 8-(1-(2,2-difluoroethyl)-1H-pyrazolo[3,4-b]pyrazin-6-yl)-1-ethyl-2-(2-(trifluoromethyl)pyridin-4-yl)-2,8-diazaspiro[4.5]decan-3-one. The crude product was purified by Prep-HPLC with the following conditions (Column: CHIRAL ART Amylose-SA, 2*25 cm, 5 μm; Mobile Phase A: Hex (10 mM NH₃-MeOH), Mobile Phase B: EtOH—HPLC; Flow rate: 25 mL/min; Gradient: 30% B to 30% B in 22 min; Wave Length: 250/203 nm; RT1(min): 10.79; RT2(min): 14.845; Sample Solvent: MeOH-HPLC; Injection Volume: 1 mL; Number Of Runs: 3) to afford (R)-8-(1-(2,2-difluoroethyl)-1H-pyrazolo[3,4-b]pyrazin-6-yl)-1-ethyl-2-(2-(trifluoromethyl)pyridin-4-yl)-2,8-diazaspiro[4.5]decan-3-one (40.4 mg, 20%) as a white solid, and (S)-8-(1-(2,2-difluoroethyl)-1H-pyrazolo[3,4-b]pyrazin-6-yl)-1-ethyl-2-(2-(trifluoromethyl)pyridin-4-yl)-2,8-diazaspiro[4.5]decan-3-one (39.8 mg, 19%) as a white solid.

### (R)-8-(1-(2,2-difluoroethyl)-1H-pyrazolo[3,4-b]pyrazin-6-yl)-1-ethyl-2-(2-(trifluoromethyl)pyridin-4-yl)-2,8-diazaspiro[4.5]decan-3-one

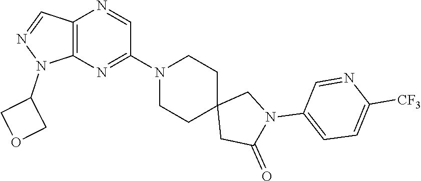 [SMILES: O=C1CC2(CCN(c3cnc4cnn(C5COC5)c4n3)CC2)CN1c1ccc(C(F)(F)F)nc1]

Step 1: tert-butyl 3-oxo-2-(6-(trifluoromethyl)pyridin-3-yl)-2,8-diazaspiro[4.5]decane-8-carboxylate: Followed the general procedure I using tert-butyl 3-oxo-2,8-diazaspiro[4.5]decane-8-carboxylate (150 mg, 588 μmol, 1.0 equiv.) and 5-fluoro-2-(trifluoromethyl)pyridine (108 mg, 647 μmol, 1.0 equiv.) as the starting materials to give tert-butyl 3-oxo-2-(6-(trifluoromethyl)pyridin-3-yl)-2,8-diazaspiro[4.5]decane-8-carboxylate (160 mg, 40%) as a white solid. MS m/z: 400 [M+H]⁺.

Step 2: 2-(6-(trifluoromethyl)pyridin-3-yl)-2,8-diazaspiro[4.5]decan-3-one: Followed the general procedure H using tert-butyl 3-oxo-2-(6-(trifluoromethyl)pyridin-3-yl)-2,8-diazaspiro[4.5]decane-8-carboxylate (160 mg) as the starting material to give the crude product 2-(6-(trifluoromethyl)pyridin-3-yl)-2,8-diazaspiro[4.5]decan-3-one. MS m/z: 300 [M+H]⁺.

Step 3: 8-(1-(oxetan-3-yl)-1H-pyrazolo[3,4-b]pyrazin-6-yl)-2-(6-(trifluoromethyl)pyridin-3-yl)-2,8-diazaspiro[4.5]decan-3-one: Followed the general procedure I using 2-(6-(trifluoromethyl)pyridin-3-yl)-2,8-diazaspiro[4.5]decan-3-one (80 mg, 266 μmol, 1.0 equiv.) and 6-chloro-1-(oxetan-3-yl)-1H-pyrazolo[3,4-b]pyrazine (62 mg, 293 μmol, 1.1 equiv.) as the starting materials to give 8-(1-(oxetan-3-yl)-1H-pyrazolo[3,4-b]pyrazin-6-yl)-2-(6-(trifluoromethyl)pyridin-3-yl)-2,8-diazaspiro[4.5]decan-3-one (84 mg, 66%) as a white solid. ¹H NMR (400 MHz, DMSO-d₆) δ 9.05 (d, J=2.5 Hz, 1H), 8.49 (d, J=1.7 Hz, 1H), 8.40 (dd, J=8.7, 2.5 Hz, 1H), 8.19 (d, J=1.6 Hz, 1H), 7.94 (d, J=8.6 Hz, 1H), 5.91-5.62 (m, J=7.2 Hz, 1H), 5.05 (t, J=6.4 Hz, 2H), 4.97 (t, J=7.2 Hz, 2H), 3.92 (dd, J=13.8, 5.9 Hz, 4H), 3.76 (dt, J=13.2, 5.7 Hz, 2H), 2.64 (s, 2H), 1.75 (t, J=5.6 Hz, 4H). MS m/z: 474.05 [M+H]⁺.

### Example 857: 8-(1-(oxetan-3-yl)-1H-pyrazolo[3,4-b]pyrazin-6-yl)-2-(5-(trifluoromethyl)pyridin-2-yl)-2,8-diazaspiro[4.5]decan-3-one

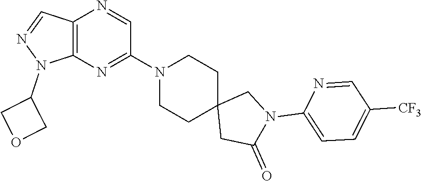 [SMILES: O=C1CC2(CCN(c3cnc4cnn(C5COC5)c4n3)CC2)CN1c1ccc(C(F)(F)F)cn1]

Step 1: tert-butyl 3-oxo-2-(5-(trifluoromethyl)pyridin-2-yl)-2,8-diazaspiro[4.5]decane-8-carboxylate: Followed the general procedure K using tert-butyl 3-oxo-2,8-diazaspiro[4.5]decane-8-carboxylate (160 mg, 0.63 mmol, 1.0 equiv.) and 2-fluoro-5-(trifluoromethyl)pyridine (114 mg, 0.693 mmol, 1.1 equiv.) as the starting materials to give tert-butyl 3-oxo-2-(5-(trifluoromethyl)pyridin-2-yl)-2,8-diazaspiro[4.5]decane-8-carboxylate (110 mg, 44%) as a colorless oil. MS m/z: 400[M+H]⁺.

Step 2: 2-(5-(trifluoromethyl)pyridin-2-yl)-2,8-diazaspiro[4.5]decan-3-one hydrochloride: Followed the general procedure B using tert-butyl 3-oxo-2-(5-(trifluoromethyl)pyridin-2-yl)-2,8-diazaspiro[4.5]decane-8-carboxylate (80 mg) as the starting material to give the crude product 2-(5-(trifluoromethyl)pyridin-2-yl)-2,8-diazaspiro[4.5]decan-3-one hydrochloride (80 mg). MS m/z: 300 [M+H]⁺.

Step 3: 8-(1-(oxetan-3-yl)-1H-pyrazolo[3,4-b]pyrazin-6-yl)-2-(5-(trifluoromethyl)pyridin-2-yl)-2,8-diazaspiro[4.5]decan-3-one: Followed the general procedure I using 2-(5-(trifluoromethyl)pyridin-2-yl)-2,8-diazaspiro[4.5]decan-3-one hydrochloride (80 mg, 0.267 mmol, 1.0 equiv.) and 6-chloro-1-(oxetan-3-yl)-1H-pyrazolo[3,4-b]pyrazine (61.8 mg, 0.294 mmol, 1.1 equiv.) as the starting materials to give 8-(1-(oxetan-3-yl)-1H-pyrazolo[3,4-b]pyrazin-6-yl)-2-(5-(trifluoromethyl)pyridin-2-yl)-2,8-diazaspiro[4.5]decan-3-one (54.8 mg, 43%) as a yellow solid. ¹H NMR (400 MHz, DMSO-d₆) δ 8.77 (s, 1H), 8.58-8.44 (m, 2H), 8.26-8.15 (m, 2H), 5.98-5.84 (m, 1H), 5.09-5.02 (m, 2H), 5.00-4.92 (m, 2H), 4.02-3.91 (m, 4H), 3.75-3.59 (m, 2H), 2.72 (s, 2H), 1.81-1.66 (m, 4H). MS m/z: 474.05 [M+H]⁺.

### Example 858: 1-ethyl-8-(1-(oxetan-3-yl)-1H-pyrazolo[3,4-b]pyrazin-6-yl)-2-(6-(trifluoromethyl)pyridin-3-yl)-2,8-diazaspiro[4.5]decan-3-one

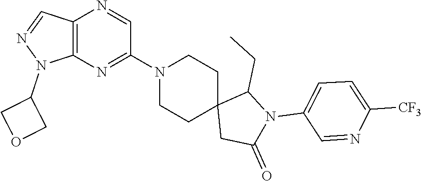 [SMILES: CCC1N(c2ccc(C(F)(F)F)nc2)C(=O)CC12CCN(c1cnc3cnn(C4COC4)c3n1)CC2]

Step 1: tert-butyl 1-ethyl-3-oxo-2-(6-(trifluoromethyl)pyridin-3-yl)-2,8-diazaspiro[4.5]decane-8-carboxylate: Followed the general procedure Y using tert-butyl 1-ethyl-3-oxo-2,8-diazaspiro[4.5]decane-8-carboxylate (100 mg, 355 μmol, 1.0 equiv.) and 5-bromo-2-(trifluoromethyl)pyridine (160 mg, 709 μmol, 2.0 equiv.) as the starting materials to give tert-butyl 1-ethyl-3-oxo-2-(6-(trifluoromethyl)pyridin-3-yl)-2,8-diazaspiro[4.5]decane-8-carboxylate (140 mg, 92%) as a yellow oil. MS m/z: 428 [M+H]⁺.

Step 2: 1-ethyl-2-(6-(trifluoromethyl)pyridin-3-yl)-2,8-diazaspiro[4.5]decan-3-one hydrochloride: Followed the general procedure B using tert-butyl 1-ethyl-3-oxo-2-(6-(trifluoromethyl)pyridin-3-yl)-2,8-diazaspiro[4.5]decane-8-carboxylate (140 mg) as the starting material to give the crude product 1-ethyl-2-(6-(trifluoromethyl)pyridin-3-yl)-2,8-diazaspiro[4.5]decan-3-one hydrochloride (110 mg) as a yellow solid. MS m/z: 328 [M+H]+.

Step 3: 1-ethyl-8-(1-(oxetan-3-yl)-1H-pyrazolo[3,4-b]pyrazin-6-yl)-2-(6-(trifluoromethyl)pyridin-3-yl)-2,8-diazaspiro[4.5]decan-3-one: Followed the general procedure I using 1-ethyl-2-(6-(trifluoromethyl)pyridin-3-yl)-2,8-diazaspiro[4.5]decan-3-one hydrochloride (110 mg, 336 μmol, 1.0 equiv.) and 6-chloro-1-(oxetan-3-yl)-1H-pyrazolo[3,4-b]pyrazine (84.8 mg, 404 μmol, 1.2 equiv.) as the starting materials to give 1-ethyl-8-(1-(oxetan-3-yl)-1H-pyrazolo[3,4-b]pyrazin-6-yl)-2-(6-(trifluoromethyl)pyridin-3-yl)-2,8-diazaspiro[4.5]decan-3-one (65.7 mg, 19%) as a white solid. ¹H NMR (400 MHz, DMSO-d₆) δ 9.06 (d, J=2.5 Hz, 1H), 8.50 (s, 1H), 8.37 (dd, J=8.7, 2.5 Hz, 1H), 8.20 (s, 1H), 7.95 (d, J=8.7 Hz, 1H), 5.95-5.86 (m, 1H), 5.06 (t, J=6.4 Hz, 2H), 4.98 (t, J=6.3 Hz, 2H), 4.35 (t, J=5.5 Hz, 1H), 4.30-4.14 (m, 2H), 3.60-3.49 (m, 1H), 3.44-3.36 (m, 1H), 2.76 (d, J=17.2 Hz, 1H), 2.61 (d, J=17.2 Hz, 1H), 1.83-1.55 (m, 6H), 0.76 (t, J=7.4 Hz, 3H). MS m/z: 502.1 [M+H]⁺.

### Example 859: 1-ethyl-8-(1-(oxetan-3-yl)-1H-pyrazolo[3,4-b]pyrazin-6-yl)-2-(5-(trifluoromethyl)pyridin-2-yl)-2,8-diazaspiro[4.5]decan-3-one

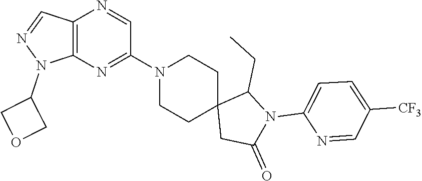 [SMILES: CCC1N(c2ccc(C(F)(F)F)cn2)C(=O)CC12CCN(c1cnc3cnn(C4COC4)c3n1)CC2]

Step 1: tert-butyl 1-ethyl-3-oxo-2-(5-(trifluoromethyl)pyridin-2-yl)-2,8-diazaspiro[4.5]decane-8-carboxylate: Followed the general procedure E using tert-butyl 1-ethyl-3-oxo-2,8-diazaspiro[4.5]decane-8-carboxylate (100 mg, 0.354 mmol, 1 equiv) and 2-bromo-5-(trifluoromethyl)pyridine (70.16 mg, 0.425 mmol, 1.2 equiv) as the starting materials to give tert-butyl 1-ethyl-3-oxo-2-(5-(trifluoromethyl)pyridin-2-yl)-2,8-diazaspiro[4.5]decane-8-carboxylate (120 mg, 65%) as a white solid. MS m/z: 428 [M+H]⁺.

Step 2: 1-ethyl-2-(5-(trifluoromethyl)pyridin-2-yl)-2,8-diazaspiro[4.5]decan-3-on hydrochloride: Followed the general procedure B using tert-butyl 1-ethyl-3-oxo-2,8-diazaspiro[4.5]decane-8-carboxylate (120 mg) as the starting material to give the crude product 1-ethyl-2-(5-(trifluoromethyl)pyridin-2-yl)-2,8-diazaspiro[4.5]decan-3-one hydrochloride (100 mg). MS m/z: 328 [M+H]⁺.

Step 3: 1-ethyl-8-(1-(oxetan-3-yl)-1H-pyrazolo[3,4-b]pyrazin-6-yl)-2-(5-(trifluoromethyl)pyridin-2-yl)-2,8-diazaspiro[4.5]decan-3-one: Followed the general procedure I using 1-ethyl-2-[5-(trifluoromethyl)pyridin-2-yl]-2,8-diazaspiro[4.5]decan-3-one hydrochloride (100 mg, 0.305 mmol, 1 equiv) and 6-chloro-1-(oxetan-3-yl)-1H-pyrazolo[3,4-b]pyrazine (77 mg, 0.366 mmol, 1.2 equiv) as the starting materials to give 1-ethyl-8-(1-(oxetan-3-yl)-1H-pyrazolo[3,4-b]pyrazin-6-yl)-2-(5-(trifluoromethyl)pyridin-2-yl)-2,8-diazaspiro[4.5]decan-3-one (90 mg, 58%) as a white solid. ¹H NMR (400 MHz, DMSO-d₆) δ 8.77 (s, 1H), 8.53-8.44 (m, 2H), 8.26-8.16 (m, 2H), 5.95-5.84 (m, 1H), 5.05 (t, J=6.4 Hz, 2H), 4.97 (t, J=7.1 Hz, 2H), 4.61 (t, J=5.4 Hz, 1H), 4.27-4.11 (m, 2H), 3.58-3.46 (m, 1H), 3.40-3.33 (m, 1H), 2.88-2.79 (m, 1H), 2.74-2.65 (m, 1H), 2.03-1.87 (m, 1H), 1.85-1.78 (m, 2H), 1.73-1.60 (m, 3H), 0.78 (t, J=7.4 Hz, 3H). MS m/z: 502.10 [M+H]⁺.

### Example 860: 3-(1-(2,2-difluoroethyl)-1H-pyrazolo[3,4-b]pyrazin-6-yl)-1-ethyl-8-(5-(trifluoromethyl)pyridin-2-yl)-1,3,8-triazaspiro[4.5]decane-2,4-dione

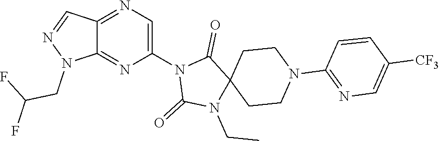 [SMILES: CCN1C(=O)N(c2cnc3cnn(CC(F)F)c3n2)C(=O)C12CCN(c1ccc(C(F)(F)F)cn1)CC2]

Step 1: tert-butyl 3-(1-(2,2-difluoroethyl)-1H-pyrazolo[3,4-b]pyrazin-6-yl)-2,4-dioxo-1,3,8-triazaspiro[4.5]decane-8-carboxylate: Followed the general procedure I using tert-butyl 2,4-dioxo-1,3,8-triazaspiro[4.5]decane-8-carboxylate (300 mg, 1.11 mmol, 1.0 equiv.) and 6-chloro-1-(2,2-difluoroethyl)-1H-pyrazolo[3,4-b]pyrazine (242 mg, 1.11 mmol, 1.0 equiv.) as the starting materials to give tert-butyl 3-(1-(2,2-difluoroethyl)-1H-pyrazolo[3,4-b]pyrazin-6-yl)-2,4-dioxo-1,3,8-triazaspiro[4.5]decane-8-carboxylate (230 mg, 46%) as a yellow oil. MS m/z: 452 [M+H]⁺.

Step 2: tert-butyl 3-(1-(2,2-difluoroethyl)-1H-pyrazolo[3,4-b]pyrazin-6-yl)-1-ethyl-2,4-dioxo-1,3,8-triazaspiro[4.5]decane-8-carboxylate: Followed the general procedure E using tert-butyl 3-(1-(2,2-difluoroethyl)-1H-pyrazolo[3,4-b]pyrazin-6-yl)-2,4-dioxo-1,3,8-triazaspiro[4.5]decane-8-carboxylate (220 mg, 0.488 mmol, 1.0 equiv.) and iodoethane (114 mg, 0.732 mmol, 1.5 equiv.) as the starting materials to give tert-butyl 3-(1-(2,2-difluoroethyl)-1H-pyrazolo[3,4-b]pyrazin-6-yl)-1-ethyl-2,4-dioxo-1,3,8-triazaspiro[4.5]decane-8-carboxylate (170 mg, 73%) as a yellow oil. MS m/z: 480 [M+H]⁺.

Step 3: 3-(1-(2,2-difluoroethyl)-1H-pyrazolo[3,4-b]pyrazin-6-yl)-1-ethyl-1,3,8-triazaspiro[4.5]decane-2,4-dione hydrochloride: Followed the general procedure B using tert-butyl 3-(1-(2,2-difluoroethyl)-1H-pyrazolo[3,4-b]pyrazin-6-yl)-1-ethyl-2,4-dioxo-1,3,8-triazaspiro[4.5]decane-8-carboxylate (170 mg) as the starting material to give the crude product 3-(1-(2,2-difluoroethyl)-1H-pyrazolo[3,4-b]pyrazin-6-yl)-1-ethyl-1,3,8-triazaspiro[4.5]decane-2,4-dione hydrochloride (130 mg) as a yellow solid. MS m/z: 380 [M+H]⁺.

Step 4: 3-(1-(2,2-difluoroethyl)-1H-pyrazolo[3,4-b]pyrazin-6-yl)-1-ethyl-8-(5-(trifluoromethyl)pyridin-2-yl)-1,3,8-triazaspiro[4.5]decane-2,4-dione: Followed the general procedure I using 3-(1-(2,2-difluoroethyl)-1H-pyrazolo[3,4-b]pyrazin-6-yl)-1-ethyl-1,3,8-triazaspiro[4.5]decane-2,4-dione (65 mg, 0.172 mmol, 1.0 equiv.) and 2-fluoro-5-(trifluoromethyl)pyridine (42.4 mg, 0.257 mmol, 1.5 equiv.) as the starting materials to give 3-(1-(2,2-difluoroethyl)-1H-pyrazolo[3,4-b]pyrazin-6-yl)-1-ethyl-8-(5-(trifluoromethyl)pyridin-2-yl)-1,3,8-triazaspiro[4.5]decane-2,4-dione (40 mg, 44%) as a white solid. ¹H NMR (400 MHz, DMSO-d₆) δ 8.88 (s, 1H), 8.70 (s, 1H), 8.43 (d, J=2.6 Hz, 1H), 7.89-7.75 (m, 1H), 7.06 (d, J=9.1 Hz, 1H), 6.76-6.29 (m, 1H), 5.06-4.92 (m, 2H), 4.53 (d, J=13.6 Hz, 2H), 3.62-3.47 (m, 2H), 3.35 (q, J=7.1 Hz, 2H), 2.18-2.01 (m, 4H), 1.18 (t, J=7.0 Hz, 3H). MS m/z: 525.2 [M+H]⁺.

### Example 861: 3-(1-(2,2-difluoroethyl)-1H-pyrazolo[3,4-b]pyrazin-6-yl)-1-ethyl-8-(6-(trifluoromethyl)pyridin-3-yl)-1,3,8-triazaspiro[4.5]decane-2,4-dione

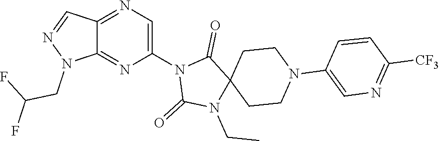 [SMILES: CCN1C(=O)N(c2cnc3cnn(CC(F)F)c3n2)C(=O)C12CCN(c1ccc(C(F)(F)F)nc1)CC2]

Step 1: 3-(1-(2,2-difluoroethyl)-1H-pyrazolo[3,4-b]pyrazin-6-yl)-1-ethyl-8-(6-(trifluoromethyl)pyridin-3-yl)-1,3,8-triazaspiro[4.5]decane-2,4-dione: Followed the general procedure I using 3-[1-(2,2-difluoroethyl)pyrazolo[3,4-b]pyrazin-6-yl]-1-ethyl-1,3,8-triazaspiro[4.5]decane-2,4-dione hydrochloride (50 mg, 0.13 mmol, 1 equiv.) and 5-bromo-2-(trifluoromethyl)pyridine (59.6 mg, 0.264 mmol, 2 equiv.) as the starting materials to give 3-(1-(2,2-difluoroethyl)-1H-pyrazolo[3,4-b]pyrazin-6-yl)-1-ethyl-8-(6-(trifluoromethyl)pyridin-3-yl)-1,3,8-triazaspiro[4.5]decane-2,4-dione (10.5 mg, 15%) as a white solid. ¹H NMR (400 MHz, Chloroform-d) δ 8.76 (s, 1H), 8.42 (d, J=7.1 Hz, 2H), 7.57 (d, J=8.7 Hz, 1H), 7.29 (d, J=3.0 Hz, 1H), 6.44-6.12 (m, 1H), 4.97-4.79 (m, 2H), 3.88 (d, J=12.3 Hz, 2H), 3.77 (t, J=12.2 Hz, 2H), 3.48-3.39 (m, 2H), 2.29-2.18 (m, 2H), 2.10 (d, J=13.4 Hz, 2H), 1.36 (t, J=7.1 Hz, 3H). MS m/z: 525.2 [M+H]⁺.

### Example 862: 8-(1-(oxetan-3-yl)-1H-pyrazolo[3,4-b]pyrazin-6-yl)-2-(5-(trifluoromethyl)pyrazin-2-yl)-2,8-diazaspiro[4.5]decan-3-one

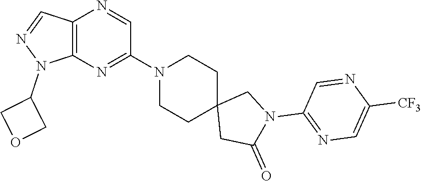 [SMILES: O=C1CC2(CCN(c3cnc4cnn(C5COC5)c4n3)CC2)CN1c1cnc(C(F)(F)F)cn1]

Step 1: tert-butyl 3-oxo-2-(5-(trifluoromethyl)pyrazin-2-yl)-2,8-diazaspiro[4.5]decane-8-carboxylate: Followed the general procedure I using tert-butyl 3-oxo-2,8-diazaspiro[4.5]decane-8-carboxylate (160 mg, 627 μmol, 1.0 equiv.) and 2-chloro-5-(trifluoromethyl)pyrazine (127 mg, 690 μmol, 1.1 equiv.) as the starting materials to give tert-butyl 3-oxo-2-(5-(trifluoromethyl)pyrazin-2-yl)-2,8-diazaspiro[4.5]decane-8-carboxylate (110 mg, 43%) as a white solid. MS m/z: 401 [M+H]⁺.

Step 2: 2-(5-(trifluoromethyl)pyrazin-2-yl)-2,8-diazaspiro[4.5]decan-3-one: Followed the general procedure H using tert-butyl 3-oxo-2-(5-(trifluoromethyl)pyrazin-2-yl)-2,8-diazaspiro[4.5]decane-8-carboxylate (110 mg) as the starting material to give the crude product 2-(5-(trifluoromethyl)pyrazin-2-yl)-2,8-diazaspiro[4.5]decan-3-one (80 mg). MS m/z: 301 [M+H]⁺.

Step 3: 8-(1-(oxetan-3-yl)-1H-pyrazolo[3,4-b]pyrazin-6-yl)-2-(5-(trifluoromethyl)pyrazin-2-yl)-2,8-diazaspiro[4.5]decan-3-one: Followed the general procedure I using 2-(5-(trifluoromethyl)pyrazin-2-yl)-2,8-diazaspiro[4.5]decan-3-one (80 mg, 265 μmol, 1.0 equiv.) and 6-chloro-1-(oxetan-3-yl)-1H-pyrazolo[3,4-b]pyrazine (62 mg, 292 μmol, 1.1 equiv.) as the starting materials to give 8-(1-(oxetan-3-yl)-1H-pyrazolo[3,4-b]pyrazin-6-yl)-2-(5-(trifluoromethyl)pyrazin-2-yl)-2,8-diazaspiro[4.5]decan-3-one (28.7 mg, 22%) as a white solid. ¹H NMR (400 MHz, DMSO-d₆) δ 9.60 (d, J=1.5 Hz, 1H), 8.89 (d, J=1.5 Hz, 1H), 8.41 (s, 1H), 8.11 (s, 1H), 5.83-5.62 (m, J=7.1 Hz, 1H), 4.98 (t, J=6.4 Hz, 2H), 4.90 (dd, J=7.9, 6.3 Hz, 2H), 3.93-3.81 (m, 4H), 3.67-3.56 (m, 2H), 2.68 (s, 2H), 1.70 (dt, J=7.1, 4.0 Hz, 4H). MS m/z: 475.20 [M+H]⁺.

### Example 863: (2R)-5-(1-(2,2-difluoroethyl)-1H-pyrazolo[3,4-b]pyrazin-6-yl)-1′-(5-(trifluoromethyl)pyridin-2-yl)-5-azaspiro[bicyclo[2.2.2]octane-2,3′-pyrrolidin]-5′-one, assumed

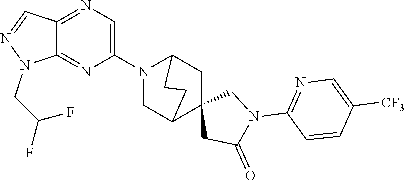 [SMILES: O=C1C[C@@]2(CC3CCC2CN3c2cnc3cnn(CC(F)F)c3n2)CN1c1ccc(C(F)(F)F)cn1]

Step 1: tert-butyl (2R)-5′-oxo-1′-(5-(trifluoromethyl)pyridin-2-yl)-5-azaspiro[bicyclo[2.2.2]octane-2,3′-pyrrolidine]-5-carboxylate, assumed: Followed the general procedure Y using tert-butyl 5′-oxo-5-azaspiro[bicyclo[2.2.2]octane-2,3′-pyrrolidine]-5-carboxylate (160 mg, 825 μmol, 1.0 equiv.) and 2-fluoro-5-(trifluoromethyl)pyridine (258 mg, 825 μmol, 2.0 equiv.) as the starting materials to give tert-butyl (2R)-5′-oxo-1′-(5-(trifluoromethyl)pyridin-2-yl)-5-azaspiro[bicyclo[2.2.2]octane-2,3′-pyrrolidine]-5-carboxylate (100 mg, 61%) as a yellow oil. MS m/z: 426 [M+H]⁺.

Step 2: (2R)-1′-(5-(trifluoromethyl)pyridin-2-yl)-5-azaspiro[bicyclo[2.2.2]octane-2,3′-pyrrolidin]-5′-one hydrochloride, assumed: Followed the general procedure B using tert-butyl (2R)-5′-oxo-1′-(5-(trifluoromethyl)pyridin-2-yl)-5-azaspiro[bicyclo[2.2.2]octane-2,3′-pyrrolidine]-5-carboxylate (100 mg,) as the starting material to give the crude product (2R)-1′-(5-(trifluoromethyl)pyridin-2-yl)-5-azaspiro[bicyclo[2.2.2]octane-2,3′-pyrrolidin]-5′-one hydrochloride (80 mg). MS m/z: 326 [M+H]⁺.

Step 3: (2R)-5-(1-(2,2-difluoroethyl)-1H-pyrazolo[3,4-b]pyrazin-6-yl)-1′-(5-(trifluoromethyl)pyridin-2-yl)-5-azaspiro[bicyclo[2.2.2]octane-2,3′-pyrrolidin]-5′-one, assumed: Followed the general procedure I using (2R)-1′-(5-(trifluoromethyl)pyridin-2-yl)-5-azaspiro[bicyclo[2.2.2]octane-2,3′-pyrrolidin]-5′-one hydrochloride (80 mg, 0.25 mmol, 1.0 equiv.) and 6-chloro-1-(2,2-difluoroethyl)-1H-pyrazolo[3,4-b]pyrazine (64.5 mg, 0.295 mmol, 1.2 equiv.) as the starting materials to give (2R)-5-(1-(2,2-difluoroethyl)-1H-pyrazolo[3,4-b]pyrazin-6-yl)-1′-(5-(trifluoromethyl)pyridin-2-yl)-5-azaspiro[bicyclo[2.2.2]octane-2,3′-pyrrolidin]-5′-one (47.5 mg, 38%) as a white solid. ¹H NMR (400 MHz, DMSO-d₆) δ 8.70 (d, J=2.5 Hz, 1H), 8.47 (d, J=8.9 Hz, 1H), 8.32-8.15 (m, 2H), 8.10 (s, 1H), 6.60-6.27 (m, 1H), 4.82-4.58 (m, 3H), 3.98 (s, 2H), 3.81 (d, J=11.6 Hz, 1H), 3.53 (d, J=11.8 Hz, 1H), 2.93 (d, J=16.5 Hz, 1H), 2.69 (d, J=16.0 Hz, 1H), 2.12 (s, 1H), 1.99 (d, J=13.6 Hz, 1H), 1.85 (d, J=13.6 Hz, 4H), 1.72 (d, J=12.6 Hz, 1H), 1.24 (s, 1H). MS m/z: 508.2 [M+H]⁺.

### Example 864: (2S)-5-(1-(2,2-difluoroethyl)-1H-pyrazolo[3,4-b]pyrazin-6-yl)-1′-(5-(trifluoromethyl)pyridin-2-yl)-5-azaspiro[bicyclo[2.2.2]octane-2,3′-pyrrolidin]-5′-one, assumed

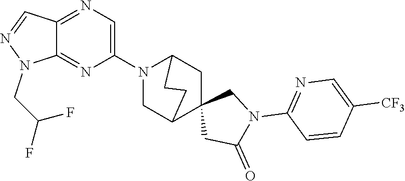 [SMILES: O=C1C[C@]2(CC3CCC2CN3c2cnc3cnn(CC(F)F)c3n2)CN1c1ccc(C(F)(F)F)cn1]

Step 1: tert-butyl (2S)-5′-oxo-1′-(5-(trifluoromethyl)pyridin-2-yl)-5-azaspiro[bicyclo[2.2.2]octane-2,3′-pyrrolidine]-5-carboxylate, assumed: Followed the general procedure Y using tert-butyl 5′-oxo-5-azaspiro[bicyclo[2.2.2]octane-2,3′-pyrrolidine]-5-carboxylate (160 mg, 825 μmol, 1.0 equiv.) and 2-fluoro-5-(trifluoromethyl)pyridine (258 mg, 825 μmol, 2.0 equiv.) as the starting materials to give tert-butyl (2S)-5′-oxo-1′-(5-(trifluoromethyl)pyridin-2-yl)-5-azaspiro[bicyclo[2.2.2]octane-2,3′-pyrrolidine]-5-carboxylate (51 mg, 61%) as a yellow oil. MS m/z: 426 [M+H]⁺.

Step 2: (2S)-1′-(5-(trifluoromethyl)pyridin-2-yl)-5-azaspiro[bicyclo[2.2.2]octane-2,3′-pyrrolidin]-5′-one hydrochloride, assumed: Followed the general procedure B using tert-butyl (2S)-5′-oxo-1′-(5-(trifluoromethyl)pyridin-2-yl)-5-azaspiro[bicyclo[2.2.2]octane-2,3′-pyrrolidine]-5-carboxylate (50 mg) as the starting material to give the crude product (2S)-1′-(5-(trifluoromethyl)pyridin-2-yl)-5-azaspiro[bicyclo[2.2.2]octane-2,3′-pyrrolidin]-5′-one hydrochloride (38 mg). MS m/z: 326 [M+H]⁺.

Step 3: (2S)-5-(1-(2,2-difluoroethyl)-1H-pyrazolo[3,4-b]pyrazin-6-yl)-1′-(5-(trifluoromethyl)pyridin-2-yl)-5-azaspiro[bicyclo[2.2.2]octane-2,3′-pyrrolidin]-5′-one, assumed: Followed the general procedure I using (2S)-1′-(5-(trifluoromethyl)pyridin-2-yl)-5-azaspiro[bicyclo[2.2.2]octane-2,3′-pyrrolidin]-5′-one hydrochloride (40 mg, 0.123 mmol, 1.0 equiv.) and 6-chloro-1-(2,2-difluoroethyl)-1H-pyrazolo[3,4-b]pyrazine (32.2 mg, 0.148 mmol, 1.2 equiv.) as the starting materials to give (2S)-5-(1-(2,2-difluoroethyl)-1H-pyrazolo[3,4-b]pyrazin-6-yl)-1′-(5-(trifluoromethyl)pyridin-2-yl)-5-azaspiro[bicyclo[2.2.2]octane-2,3′-pyrrolidin]-5′-one (25 mg, 39%) as a white solid. ¹H NMR (400 MHz, DMSO-d₆) δ 8.80 (d, J=2.3 Hz, 1H), 8.47 (d, J=8.9 Hz, 1H), 8.26-8.06 (m, 3H), 6.44 (t, J=55.0 Hz, 1H), 4.84-4.54 (m, 3H), 4.16 (d, J=10.7 Hz, 1H), 4.02 (d, J=10.8 Hz, 1H), 3.72 (d, J=11.4 Hz, 1H), 3.57 (d, J=11.8 Hz, 1H), 2.91 (d, J=16.5 Hz, 1H), 2.11 (s, 1H), 1.99-1.80 (m, 5H), 1.71 (d, J=13.3 Hz, 1H). MS m/z: 508.2 [M+H]⁺.

### Example 865: 5-(1-(2,2-difluoroethyl)-1H-pyrazolo[3,4-b]pyrazin-6-yl)-1′-(6-(trifluoromethyl)pyridin-3-yl)-5-azaspiro[bicyclo[2.2.2]octane-2,3′-pyrrolidin]-5′-one

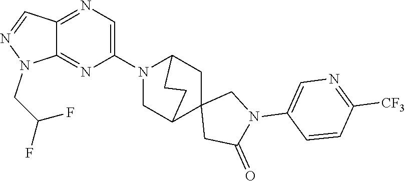 [SMILES: O=C1CC2(CC3CCC2CN3c2cnc3cnn(CC(F)F)c3n2)CN1c1ccc(C(F)(F)F)nc1]

Step 1: tert-butyl 5′-oxo-1′-(6-(trifluoromethyl)pyridin-3-yl)-5-azaspiro[bicyclo[2.2.2]octane-2,3′-pyrrolidine]-5-carboxylate: Followed the general procedure Y using tert-butyl 5′-oxo-5-azaspiro[bicyclo[2.2.2]octane-2,3′-pyrrolidine]-5-carboxylate (100 mg, 0.357 mmol, 1 equiv), and 5-bromo-2-(trifluoromethyl)pyridine (96.7 mg, 0.428 mmol, 1.2 equiv) as the starting materials, XPhos Pd G3 (30.2 mg, 0.036 mmol, 0.1 equiv) as the catalyst to give tert-butyl 5′-oxo-1′-(6-(trifluoromethyl)pyridin-3-yl)-5-azaspiro[bicyclo[2.2.2]octane-2,3′-pyrrolidine]-5-carboxylate (100 mg, 65%) as a white solid. MS m/z: 426 [M+H]⁺.

Step 2: 1′-(6-(trifluoromethyl)pyridin-3-yl)-5-azaspiro[bicyclo[2.2.2]octane-2,3′-pyrrolidin]-5′-one hydrochloride: Followed the general procedure B using tert-butyl 5′-oxo-1′-(6-(trifluoromethyl)pyridin-3-yl)-5-azaspiro[bicyclo[2.2.2]octane-2,3′-pyrrolidine]-5-carboxylate (100 mg) as the starting material to give the crude product 1′-(6-(trifluoromethyl)pyridin-3-yl)-5-azaspiro[bicyclo[2.2.2]octane-2,3′-pyrrolidin]-5′-one hydrochloride (70 mg). MS m/z: 326 [M+H]⁺.

Step 3: 5-(1-(2,2-difluoroethyl)-1H-pyrazolo[3,4-b]pyrazin-6-yl)-1′-(6-(trifluoromethyl)pyridin-3-yl)-5-azaspiro[bicyclo[2.2.2]octane-2,3′-pyrrolidin]-5′-one: Followed the general procedure I using 1′-(6-(trifluoromethyl)pyridin-3-yl)-5-azaspiro[bicyclo[2.2.2]octane-2,3′-pyrrolidin]-5′-one hydrochloride (66 mg, 0.203 mmol, 1 equiv) and 6-chloro-1-(2,2-difluoroethyl)-1H-pyrazolo[3,4-b]pyrazine (53.21 mg, 0.244 mmol, 1.2 equiv) as the starting materials to give 5-(1-(2,2-difluoroethyl)-1H-pyrazolo[3,4-b]pyrazin-6-yl)-1′-(6-(trifluoromethyl)pyridin-3-yl)-5-azaspiro[bicyclo[2.2.2]octane-2,3′-pyrrolidin]-5′-one (30 mg, 29%) as a white solid. ¹H NMR (400 MHz, DMSO-d₆) δ 9.10-9.00 (m, 1H), 8.44-8.30 (m, 1H), 8.29-8.11 (m, 1H), 8.11-8.03 (m, 1H), 7.98-7.86 (m, 1H), 6.44 (t, 1H), 5.02-4.73 (m, 1H), 4.71-4.58 (m, 2H), 4.00 (d, J=9.4 Hz, 1H), 3.93-3.65 (m, 2H), 3.61-3.53 (m, 1H), 2.89-2.79 (m, 1H), 2.67-2.58 (m, 1H), 2.14-2.06 (m, 1H), 2.04-1.96 (m, 1H), 1.93-1.77 (m, 4H), 1.75-1.65 (m, 1H). MS m/z: 508.20 [M+H]⁺.

### Example 866: 1-ethyl-8-(1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione

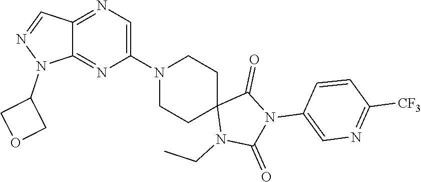 [SMILES: CCN1C(=O)N(c2ccc(C(F)(F)F)nc2)C(=O)C12CCN(c1cnc3cnn(C4COC4)c3n1)CC2]

Step 1: 1-ethyl-8-(1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione: Followed the general procedure I using 1-ethyl-3-(6-(trifluoromethyl)pyridin-3-yl)-1,3,8-triazaspiro[4.5]decane-2,4-dione (50 mg, 146 μmol, 1.0 equiv.) and 6-chloro-1-(oxetan-3-yl)-1H-pyrazolo[3,4-b]pyrazine (33.8 mg, 161 μmol, 1.1 equiv.) as the starting materials to give 1-ethyl-8-(1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione (25 mg, 26%) as a white solid. ¹H NMR (400 MHz, DMSO-d₆) δ 8.92 (d, J=2.3 Hz, 1H), 8.52 (s, 1H), 8.26 (dd, J=8.4, 2.3 Hz, 1H), 8.21 (s, 1H), 8.12 (d, J=8.5 Hz, 1H), 5.99-5.88 (m, 1H), 5.07 (t, J=6.4 Hz, 2H), 4.98 (t, J=6.3 Hz, 2H), 4.64 (d, J=13.5 Hz, 2H), 3.70-3.56 (m, 2H), 3.38-3.32 (m, 2H), 2.20-2.06 (m, 4H), 1.16 (t, J=7.0 Hz, 3H). MS m/z: 517.1 [M+H]⁺.

### Example 867: 1-isopropyl-8-(1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione

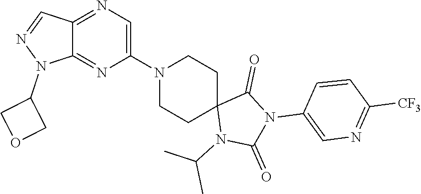 [SMILES: CC(C)N1C(=O)N(c2ccc(C(F)(F)F)nc2)C(=O)C12CCN(c1cnc3cnn(C4COC4)c3n1)CC2]

Step 1: tert-butyl 1-isopropyl-2,4-dioxo-3-(6-(trifluoromethyl)pyridin-3-yl)-1,3,8-triazaspiro[4.5]decane-8-carboxylate: Followed the general procedure I using tert-butyl 2,4-dioxo-3-(6-(trifluoromethyl)pyridin-3-yl)-1,3,8-triazaspiro[4.5]decane-8-carboxylate (150 mg, 361 μmol, 1.0 equiv.) and 2-iodopropane (68 mg, 397 μmol, 1.1 equiv.) as the starting materials to give tert-butyl 1-isopropyl-2,4-dioxo-3-(6-(trifluoromethyl)pyridin-3-yl)-1,3,8-triazaspiro[4.5]decane-8-carboxylate (110 mg, 66%) as a white solid. MS m/z: 457 [M+H]⁺.

Step 2: 1-isopropyl-3-(6-(trifluoromethyl)pyridin-3-yl)-1,3,8-triazaspiro[4.5]decane-2,4-dione: Followed the general procedure H using tert-butyl 1-isopropyl-2,4-dioxo-3-(6-(trifluoromethyl)pyridin-3-yl)-1,3,8-triazaspiro[4.5]decane-8-carboxylate (110 mg) as the starting material to give the crude product 1-isopropyl-3-(6-(trifluoromethyl)pyridin-3-yl)-1,3,8-triazaspiro[4.5]decane-2,4-dione (80 mg). MS m/z: 357 [M+H]⁺.

Step 3: 1-isopropyl-8-(1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione: Followed the general procedure I using 1-isopropyl-3-(6-(trifluoromethyl)pyridin-3-yl)-1,3,8-triazaspiro[4.5]decane-2,4-dione (80 mg, 224 μmol, 1.0 equiv.) and 6-chloro-1-(oxetan-3-yl)-1H-pyrazolo[3,4-b]pyrazine (52 mg, 246 μmol, 1.1 equiv.) as the starting materials to give 1-isopropyl-8-(1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione (43.4 mg, 36%) as a white solid. ¹H NMR (400 MHz, DMSO-d₆) δ 8.91 (d, J=2.3 Hz, 1H), 8.52 (d, J=1.7 Hz, 1H), 8.29-8.19 (m, 2H), 8.11 (dd, J=8.5, 1.7 Hz, 1H), 5.94-5.67 (td, J=7.1, 6.5, 1.6 Hz, 1H), 5.07 (td, J=6.5, 1.6 Hz, 2H), 4.99 (ddd, J=8.0, 6.4, 1.6 Hz, 2H), 4.70-4.61 (m, 2H), 3.63 (t, J=12.9 Hz, 3H), 2.17 (d, J=13.1 Hz, 2H), 2.06 (td, J=13.2, 4.5 Hz, 2H), 1.35 (dd, J=6.7, 1.7 Hz, 5H), 1.24 (d, J=1.8 Hz, 1H). MS m/z: 531.20 [M+H]⁺.

### Example 868: 8-(1-(2,2-difluoroethyl)-1H-pyrazolo[3,4-b]pyrazin-6-yl)-1-ethyl-3-(4-(trifluoromethyl)phenyl)-1,3,8-triazaspiro[4.5]decane-2,4-dione

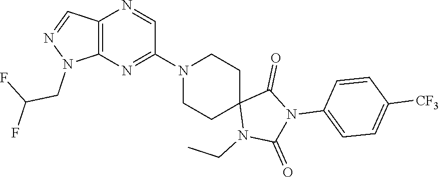 [SMILES: CCN1C(=O)N(c2ccc(C(F)(F)F)cc2)C(=O)C12CCN(c1cnc3cnn(CC(F)F)c3n1)CC2]

Step 1: 8-(1-(2,2-difluoroethyl)-1H-pyrazolo[3,4-b]pyrazin-6-yl)-1-ethyl-3-(4-(trifluoromethyl)phenyl)-1,3,8-triazaspiro[4.5]decane-2,4-dione: Followed the general procedure I using 1-ethyl-3-(4-(trifluoromethyl)phenyl)-1,3,8-triazaspiro[4.5]decane-2,4-dione (70 mg, 0.205 mmol, 1.0 equiv.) and 6-chloro-1-(2,2-difluoroethyl)-1H-pyrazolo[3,4-b]pyrazine (60 mg, 0.274 mmol, 1.3 equiv.) as the starting materials to give 8-(1-(2,2-difluoroethyl)-1H-pyrazolo[3,4-b]pyrazin-6-yl)-1-ethyl-3-(4-(trifluoromethyl)phenyl)-1,3,8-triazaspiro[4.5]decane-2,4-dione (38.2 mg, 35%) as a white solid. ¹H NMR (400 MHz, DMSO-d₆) δ 8.54 (s, 1H), 8.16 (s, 1H), 7.90 (d, J=8.4 Hz, 2H), 7.73 (d, J=8.4 Hz, 2H), 6.45 (t, J=3.8 Hz, 1H), 4.79-4.67 (m, 2H), 4.64 (d, J=13.5 Hz, 2H), 3.70-3.58 (m, 2H), 3.33 (s, 1H), 3.30 (s, 1H), 2.15-2.05 (m, 4H), 1.15 (t, J=7.0 Hz, 3H). MS m/z: 524.05 [M+H]⁺.

### Example 869: 1-ethyl-8-(1-(oxetan-3-yl)-1H-pyrazolo[3,4-b]pyrazin-6-yl)-3-(4-(trifluoromethyl)phenyl)-1,3,8-triazaspiro[4.5]decane-2,4-dione

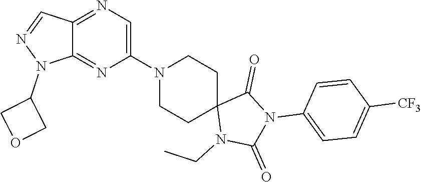 [SMILES: CCN1C(=O)N(c2ccc(C(F)(F)F)cc2)C(=O)C12CCN(c1cnc3cnn(C4COC4)c3n1)CC2]

Step 1: tert-butyl 4-(((benzyloxy)carbonyl)amino)-4-((4-(trifluoromethyl)phenyl)carbamoyl)piperidine-1-carboxylate: To a stirred mixture of 4-(((benzyloxy)carbonyl)amino)-1-(tert-butoxycarbonyl)piperidine-4-carboxylic acid (1 g, 0.132 mmol, 1.0 equiv.) and 4-(trifluoromethyl) aniline (410 mg, 0.132 mmol, 1.0 equiv.) in DMF (5 mL) were added TCFH (280 mg, 0.264 mmol, 2.0 equiv.) and NMI (1.3 g, 0.396 mmol, 3.0 equiv.) The resulting mixture was stirred for 1 h at room temperature under argon atmosphere. The resulting mixture was diluted with water (10 mL). The resulting mixture was extracted with EtOAc (3×20 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, ACN in water, 0% to 100% gradient in 10 min; detector, UV 254 nm. This resulted in tert-butyl 4-(((benzyloxy)carbonyl)amino)-4-((4-(trifluoromethyl)phenyl)carbamoyl)piperidine-1-carboxylate (600 mg, 56%) as a white solid. MS m/z: 522 [M+H]⁺.

Step 2: tert-butyl 2,4-dioxo-3-(4-(trifluoromethyl)phenyl)-1,3,8-triazaspiro[4.5]decane-8-carboxylate: To a stirred solution of tert-butyl 4-(((benzyloxy)carbonyl)amino)-4-((4-(trifluoromethyl)phenyl)carbamoyl)piperidine-1-carboxylate (600 mg, 0.383 mmol, 1.0 equiv.) in DMF (5 mL). The resulting mixture was stirred for 2 h at 120° C. The residue was purified by reverse flash chromatography with the following conditions (column, C18 gel; mobile phase, B phase: MeCN, A phase: water; 0% to 100% B gradient in 20 min; detector: UV 254/220 nm). This resulted in tert-butyl 2,4-dioxo-3-(4-(trifluoromethyl)phenyl)-1,3,8-triazaspiro[4.5]decane-8-carboxylate (400 mg, 43%) as a white solid. MS m/z: 414 [M+H]⁺.

Step 3: tert-butyl 1-ethyl-2,4-dioxo-3-(4-(trifluoromethyl)phenyl)-1,3,8-triazaspiro[4.5]decane-8-carboxylate: Followed the general procedure K using tert-butyl 2,4-dioxo-3-(4-(trifluoromethyl)phenyl)-1,3,8-triazaspiro[4.5]decane-8-carboxylate (400 mg, 2.17 mmol, 1.0 equiv.) and iodoethane (373 mg, 2.39 mmol, 1.1 equiv.) as the starting materials to give tert-butyl 1-ethyl-2,4-dioxo-3-(4-(trifluoromethyl)phenyl)-1,3,8-triazaspiro[4.5]decane-8-carboxylate (480 mg, 49%) as an off-white solid. MS m/z: 442 [M+H]⁺.

Step 4: 1-ethyl-3-(4-(trifluoromethyl)phenyl)-1,3,8-triazaspiro[4.5]decane-2,4-dione hydrochloride: Followed the general procedure B using tert-butyl 1-ethyl-2,4-dioxo-3-(4-(trifluoromethyl)phenyl)-1,3,8-triazaspiro[4.5]decane-8-carboxylate (480 mg) as the starting material to give the crude product 1-ethyl-3-(4-(trifluoromethyl)phenyl)-1,3,8-triazaspiro[4.5]decane-2,4-dione hydrochloride (460 mg). MS m/z: 342 [M+H]⁺.

Step 5: 1-ethyl-8-(1-(oxetan-3-yl)-1H-pyrazolo[3,4-b]pyrazin-6-yl)-3-(4-(trifluoromethyl)phenyl)-1,3,8-triazaspiro[4.5]decane-2,4-dione: Followed the general procedure I using 1-ethyl-3-(4-(trifluoromethyl)phenyl)-1,3,8-triazaspiro[4.5]decane-2,4-dione hydrochloride (100 mg, 0.293 mmol, 1.0 equiv.) and 6-chloro-1-(oxetan-3-yl)-1H-pyrazolo[3,4-b]pyrazine (68 mg, 0.322 mmol, 1.1 equiv.) as the starting materials to give 1-ethyl-8-(1-(oxetan-3-yl)-1H-pyrazolo[3,4-b]pyrazin-6-yl)-3-(4-(trifluoromethyl)phenyl)-1,3,8-triazaspiro[4.5]decane-2,4-dione (74.7 mg, 49%) as a white solid. ¹H NMR (300 MHz, DMSO-d₆) δ 8.47 (s, 1H), 8.18 (s, 1H), 7.88 (d, J=8.3 Hz, 2H), 7.70 (d, J=8.3 Hz, 2H), 5.99-5.84 (m, 1H), 5.02 (dt, J=23.4, 6.7 Hz, 4H), 4.59 (d, J=13.5 Hz, 2H), 3.71-3.67 (m, 1H), 3.62-3.53 (m, 1H), 3.36-3.23 (m, 2H), 2.13-2.01 (m, 4H), 1.14 (t, J=7.0 Hz, 3H). MS m/z: 516.1 [M+H]⁺.

### Example 870: 1-ethyl-8-(3-methyl-1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione

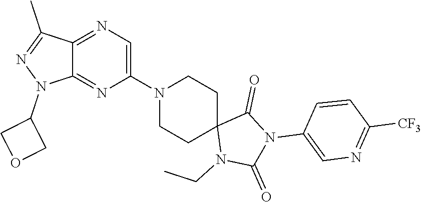 [SMILES: CCN1C(=O)N(c2ccc(C(F)(F)F)nc2)C(=O)C12CCN(c1cnc3c(C)nn(C4COC4)c3n1)CC2]

Step 1: tert-butyl 1-ethyl-2,4-dioxo-3-(6-(trifluoromethyl)pyridin-3-yl)-1,3,8-triazaspiro[4.5]decane-8-carboxylate: Followed the general procedure K using tert-butyl 2,4-dioxo-3-(6-(trifluoromethyl)pyridin-3-yl)-1,3,8-triazaspiro[4.5]decane-8-carboxylate (250 mg, 0.60 mmol, 1.0 equiv.) and EtI (188 mg, 1.2 mmol, 2.0 equiv.) as the starting materials to give tert-butyl 1-ethyl-2,4-dioxo-3-(6-(trifluoromethyl)pyridin-3-yl)-1,3,8-triazaspiro[4.5]decane-8-carboxylate (200 mg, 75%) as a white solid. MS m/z: 443 [M+H]⁺.

Step 2: 1-ethyl-3-(6-(trifluoromethyl)pyridin-3-yl)-1,3,8-triazaspiro[4.5]decane-2,4-dione hydrochloride: Followed the general procedure B using tert-butyl 1-ethyl-2,4-dioxo-3-(6-(trifluoromethyl)pyridin-3-yl)-1,3,8-triazaspiro[4.5]decane-8-carboxylate as the starting material (200 mg) to give the crude product 1-ethyl-3-(6-(trifluoromethyl)pyridin-3-yl)-1,3,8-triazaspiro[4.5]decane-2,4-dione hydrochloride (150 mg). MS m/z: 343 [M+H]⁺.

Step 3: 1-ethyl-8-(3-methyl-1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione: Followed the general procedure I using 1-ethyl-3-(6-(trifluoromethyl)pyridin-3-yl)-1,3,8-triazaspiro[4.5]decane-2,4-dione hydrochloride (70 mg, 0.18 mmol, 1.0 equiv.) and 6-chloro-3-methyl-1-(oxetan-3-yl)-1H-pyrazolo[3,4-b]pyrazine (44 mg, 0.20 mmol, 1.1 equiv.) as the starting materials to give 1-ethyl-8-(3-methyl-1-(oxetan-3-yl)-1H-pyrazolo[3,4-b]pyrazin-6-yl)-3-(6-(trifluoromethyl)pyridin-3-yl)-1,3,8-triazaspiro[4.5]decane-2,4-dione (65 mg, 67%) as a white solid. ¹H NMR (400 MHz, DMSO-d₆) δ 8.92 (d, J=2.2 Hz, 1H), 8.44 (s, 1H), 8.26 (dd, J=8.5, 2.3 Hz, 1H), 8.11 (d, J=8.5 Hz, 1H), 5.86 (p, J=7.1 Hz, 1H), 5.05 (t, J=6.4 Hz, 2H), 4.95 (t, J=7.0 Hz, 2H), 4.61 (d, J=13.6 Hz, 2H), 3.69-3.56 (m, 2H), 3.39-3.34 (m, 2H), 2.48 (s, 3H), 2.20-2.06 (m, 4H), 1.16 (t, J=7.0 Hz, 3H). MS m/z: 531.25 [M+H]⁺.

### Example 871: 8-(1-(2,2-difluoroethyl)-3-methyl-1H-pyrazolo[3,4-b]pyrazin-6-yl)-1-ethyl-3-(6-(trifluoromethyl)pyridin-3-yl)-1,3,8-triazaspiro[4.5]decane-2,4-dione

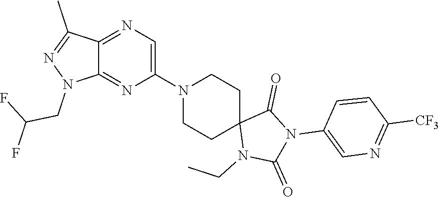 [SMILES: CCN1C(=O)N(c2ccc(C(F)(F)F)nc2)C(=O)C12CCN(c1cnc3c(C)nn(CC(F)F)c3n1)CC2]

Step 1: 8-(1-(2,2-difluoroethyl)-3-methyl-1H-pyrazolo[3,4-b]pyrazin-6-yl)-1-ethyl-3-(6-(trifluoromethyl)pyridin-3-yl)-1,3,8-triazaspiro[4.5]decane-2,4-dione: Followed the general procedure I using 1-ethyl-3-(6-(trifluoromethyl)pyridin-3-yl)-1,3,8-triazaspiro[4.5]decane-2,4-dione hydrochloride (70 mg, 0.18 mmol, 1.0 equiv.) and 6-chloro-1-(2,2-difluoroethyl)-1H-pyrazolo[3,4-b]pyrazine (44 mg, 0.198 mmol, 1.1 equiv.) as the starting materials to give 8-(1-(2,2-difluoroethyl)-3-methyl-1H-pyrazolo[3,4-b]pyrazin-6-yl)-1-ethyl-3-(6-(trifluoromethyl)pyridin-3-yl)-1,3,8-triazaspiro[4.5]decane-2,4-dione (60.5 mg, 61%) as a white solid. ¹H NMR (400 MHz, DMSO-d₆) δ 8.92 (d, J=2.3 Hz, 1H), 8.46 (s, 1H), 8.26 (dd, J=8.4, 2.3 Hz, 1H), 8.11 (d, J=8.4 Hz, 1H), 6.42 (tt, J=55.0, 3.9 Hz, 1H), 4.72-4.56 (m, 4H), 3.75-3.55 (m, 2H), 3.37-3.33 (m, 2H), 2.43 (s, 3H), 2.22-2.07 (m, 4H), 1.16 (t, J=7.0 Hz, 3H). MS m/z: 539.2 [M+H]⁺.

### Example 872: 8-(1-(2,2-difluoroethyl)-3-methyl-1H-pyrazolo[3,4-b]pyrazin-6-yl)-1-ethyl-3-(4-(trifluoromethyl)phenyl)-1,3,8-triazaspiro[4.5]decane-2,4-dione

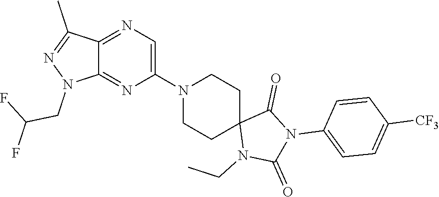 [SMILES: CCN1C(=O)N(c2ccc(C(F)(F)F)cc2)C(=O)C12CCN(c1cnc3c(C)nn(CC(F)F)c3n1)CC2]

Step 1: 8-(1-(2,2-difluoroethyl)-3-methyl-1H-pyrazolo[3,4-b]pyrazin-6-yl)-1-ethyl-3-(4-(trifluoromethyl)phenyl)-1,3,8-triazaspiro[4.5]decane-2,4-dione: Followed the general procedure I using 1-ethyl-3-(4-(trifluoromethyl)phenyl)-1,3,8-triazaspiro[4.5]decane-2,4-dione hydrochloride (100 mg, 0.293 mmol, 1.0 equiv.) and 6-chloro-1-(2,2-difluoroethyl)-3-methyl-1H-pyrazolo[3,4-b]pyrazine (75 mg, 0.322 mmol, 1.1 equiv.) as the starting materials to give 8-(1-(2,2-difluoroethyl)-3-methyl-1H-pyrazolo[3,4-b]pyrazin-6-yl)-1-ethyl-3-(4-(trifluoromethyl)phenyl)-1,3,8-triazaspiro[4.5]decane-2,4-dione (85.4 mg, 54%) as a white solid. ¹H NMR (300 MHz, DMSO-d₆) δ 8.46 (s, 1H), 7.90 (d, J=8.5 Hz, 2H), 7.74 (d, J=8.3 Hz, 2H), 6.68-6.15 (m, 1H), 4.64 (td, J=15.0, 3.9 Hz, 4H), 3.72-3.56 (m, 2H), 3.34 (s, 1H), 3.31 (s, 1H), 2.44 (s, 3H), 2.16-2.01 (m, 4H), 1.15 (t, J=7.0 Hz, 3H). MS m/z: 538.05 [M+H]⁺.

### Example 873: 8-(1-(2,2-difluoroethyl)-1H-pyrazolo[3,4-d]pyrimidin-6-yl)-1-ethyl-3-(6-(trifluoromethyl)pyridin-3-yl)-1,3,8-triazaspiro[4.5]decane-2,4-dione

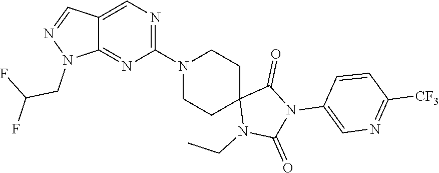 [SMILES: CCN1C(=O)N(c2ccc(C(F)(F)F)nc2)C(=O)C12CCN(c1ncc3cnn(CC(F)F)c3n1)CC2]

Step 1: 8-(1-(2,2-difluoroethyl)-1H-pyrazolo[3,4-d]pyrimidin-6-yl)-1-ethyl-3-(6-(trifluoromethyl)pyridin-3-yl)-1,3,8-triazaspiro[4.5]decane-2,4-dione: Followed the general procedure I using 1-ethyl-3-(6-(trifluoromethyl)pyridin-3-yl)-1,3,8-triazaspiro[4.5]decane-2,4-dione (60 mg, 0.175 mmol, 1 equiv) and 6-chloro-1-(2,2-difluoroethyl)-1H-pyrazolo[3,4-d]pyrimidine (45.98 mg, 0.21 mmol, 1.2 equiv) as the starting materials to give 8-(1-(2,2-difluoroethyl)-1H-pyrazolo[3,4-d]pyrimidin-6-yl)-1-ethyl-3-(6-(trifluoromethyl)pyridin-3-yl)-1,3,8-triazaspiro[4.5]decane-2,4-dione (30 mg, 32%) as a white solid. ¹H NMR (400 MHz, DMSO-d₆) δ 8.97-8.90 (m, 2H), 8.26 (dd, J=8.4, 2.3 Hz, 1H), 8.14-8.08 (m, 2H), 6.46 (tt, J=54.9, 3.8 Hz, 1H), 4.91 (d, J=13.2 Hz, 2H), 4.70 (td, J=15.0, 3.8 Hz, 2H), 3.64-3.53 (m, 2H), 3.37-3.32 (m, 2H), 2.18-2.10 (m, 2H), 2.10-1.97 (m, 2H), 1.15 (t, J=7.0 Hz, 3H). MS m/z: 525.10 [M+H]⁺.

### Example 874: 8-(1-(2,2-difluoroethyl)-1H-pyrazolo[3,4-b]pyrazin-6-yl)-1,1-dimethyl-2-(6-(trifluoromethyl)pyridin-3-yl)-2,8-diazaspiro[4.5]decan-3-one

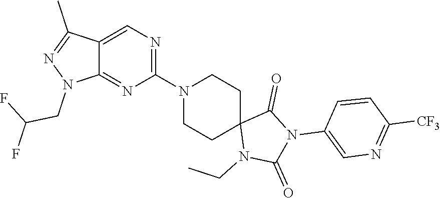 [SMILES: CCN1C(=O)N(c2ccc(C(F)(F)F)nc2)C(=O)C12CCN(c1ncc3c(C)nn(CC(F)F)c3n1)CC2]

Step 1: 8-benzyl-1,1-dimethyl-2-(6-(trifluoromethyl)pyridin-3-yl)-2,8-diazaspiro[4.5]decan-3-one: Followed the general procedure I using 1-ethyl-3-(6-(trifluoromethyl)pyridin-3-yl)-1,3,8-triazaspiro[4.5]decane-2,4-dione (30 mg, 0.09 mmol, 1.0 equiv.) and 6-chloro-1-(2,2-difluoroethyl)-3-methyl-1H-pyrazolo[3,4-d]pyrimidine (21 mg, 0.1 mmol, 1.1 equiv.) as the starting materials to give 8-(1-(2,2-difluoroethyl)-3-methyl-1H-pyrazolo[3,4-d]pyrimidin-6-yl)-1-ethyl-3-(6-(trifluoromethyl)pyridin-3-yl)-1,3,8-triazaspiro[4.5]decane-2,4-dione (15 mg, 32%) as a white solid. ¹H NMR (400 MHz, DMSO-d₆) δ 8.92 (d, J=3.1 Hz, 2H), 8.26 (dd, J=8.3, 2.3 Hz, 1H), 8.11 (d, J=8.5 Hz, 1H), 6.49 (dt, J=55.0, 3.9 Hz, 1H), 4.89 (d, J=13.2 Hz, 2H), 4.60 (td, J=15.0, 3.9 Hz, 2H), 3.67-3.50 (m, 2H), 3.36-3.32 (s, 2H), 2.44 (s, 3H), 2.14-1.99 (m, 4H), 1.15 (t, J=7.0 Hz, 3H). MS m/z: 539.05 [M+H]⁺.

### Example 875: 1-(2,2-difluoroethyl)-8-(1-(2,2-difluoroethyl)-1H-pyrazolo[3,4-d]pyrimidin-6-yl)-3-(6-(trifluoromethyl)pyridin-3-yl)-1,3,8-triazaspiro[4.5]decane-2,4-dione

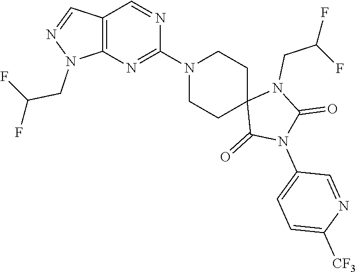 [SMILES: O=C1N(c2ccc(C(F)(F)F)nc2)C(=O)C2(CCN(c3ncc4cnn(CC(F)F)c4n3)CC2)N1CC(F)F]

Step 1: 1-(2,2-difluoroethyl)-8-(1-(2,2-difluoroethyl)-1H-pyrazolo[3,4-d]pyrimidin-6-yl)-3-(6-(trifluoromethyl)pyridin-3-yl)-1,3,8-triazaspiro[4.5]decane-2,4-dione: Followed the general procedure I using 1-(2,2-difluoroethyl)-3-(6-(trifluoromethyl)pyridin-3-yl)-1,3,8-triazaspiro[4.5]decane-2,4-dione hydrochloride (100 mg, 0.264 mmol, 1.0 equiv.) and 6-chloro-1-(2,2-difluoroethyl)-1H-pyrazolo[3,4-d]pyrimidine (69.3 mg, 0.317 mmol, 1.2 equiv.) as the starting materials to give 1-(2,2-difluoroethyl)-8-(1-(2,2-difluoroethyl)-1H-pyrazolo[3,4-d]pyrimidin-6-yl)-3-(6-(trifluoromethyl)pyridin-3-yl)-1,3,8-triazaspiro[4.5]decane-2,4-dione (22.4 mg, 15.1%) as a light yellow solid. ¹H NMR (400 MHz, DMSO-d₆) δ 9.05-8.89 (m, 2H), 8.32-8.12 (m, 2H), 8.10 (s, 1H), 6.64-6.33 (m, 1H), 6.32-6.04 (m, 1H), 4.93 (d, 2H), 4.70 (td, 2H), 3.83 (td, 2H), 3.57 (t, J=12.2 Hz, 2H), 2.20-2.01 (m, 4H). MS m/z: 561.2 [M+H]⁺.

### Example 876: 1-(2,2-difluoroethyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione

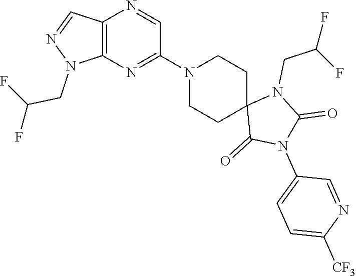 [SMILES: O=C1N(c2ccc(C(F)(F)F)nc2)C(=O)C2(CCN(c3cnc4cnn(CC(F)F)c4n3)CC2)N1CC(F)F]

Step 1: tert-butyl 1-(2,2-difluoroethyl)-2,4-dioxo-3-(6-(trifluoromethyl)pyridin-3-yl)-1,3,8-triazaspiro[4.5]decane-8-carboxylate: Followed the general procedure K using tert-butyl 2,4-dioxo-3-(2-(trifluoromethyl)pyridin-4-yl)-1,3,8-triazaspiro[4.5]decane-8-carboxylate (300 mg, 0.723 mmol, 1.0 equiv.) and 2,2-difluoroethyl trifluoromethanesulfonate (187 mg, 0.867 mmol, 1.2 equiv.) as the starting materials to give tert-butyl 1-(2,2-difluoroethyl)-2,4-dioxo-3-(6-(trifluoromethyl)pyridin-3-yl)-1,3,8-triazaspiro[4.5]decane-8-carboxylate (260 mg, 75.1%) as a light yellow oil. MS m/z: 479 [M+H]⁺.

Step 2: 1-(2,2-difluoroethyl)-3-(6-(trifluoromethyl)pyridin-3-yl)-1,3,8-triazaspiro[4.5]decane-2,4-dione hydrochloride: Followed the general procedure B using tert-butyl 1-(2,2-difluoroethyl)-2,4-dioxo-3-(6-(trifluoromethyl)pyridin-3-yl)-1,3,8-triazaspiro[4.5]decane-8-carboxylate (260 mg, 0.543 mmol, 1.0 equiv.) as the starting material to give the crude product 1-(2,2-difluoroethyl)-3-(6-(trifluoromethyl)pyridin-3-yl)-1,3,8-triazaspiro[4.5]decane-2,4-dione hydrochloride (200 mg). MS m/z: 379 [M+H]⁺.

Step 3: 1-(2,2-difluoroethyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione: Followed the general procedure I using 1-(2,2-difluoroethyl)-3-(6-(trifluoromethyl)pyridin-3-yl)-1,3,8-triazaspiro[4.5]decane-2,4-dione hydrochloride (100 mg, 0.264 mmol, 1.0 equiv.) and 6-chloro-1-(2,2-difluoroethyl)-1H-pyrazolo[3,4-b]pyrazine (69.3 mg, 0.317 mmol, 1.2 equiv.) as the starting materials to give 1-(2,2-difluoroethyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione (92.7 mg, 62.5%) as a white solid. ¹H NMR (300 MHz, DMSO-d₆) δ 8.94 (d, 1H), 8.54 (s, 1H), 8.28 (dd, 1H), 8.18-8.12 (m, 2H), 6.67-6.34 (m, 1H), 6.29-5.98 (m, 1H), 4.73 (td, 4H), 3.85 (td, 2H), 3.63 (dt, 2H), 2.23-2.12 (m, 4H). MS m/z: 561.2 [M+H]⁺.

### Example 877: 8-(1-(2,2-difluoroethyl)-1H-pyrazolo[3,4-b]pyrazin-6-yl)-1-(2-(trifluoromethoxy)ethyl)-3-(6-(trifluoromethyl)pyridin-3-yl)-1,3,8-triazaspiro[4.5]decane-2,4-dione

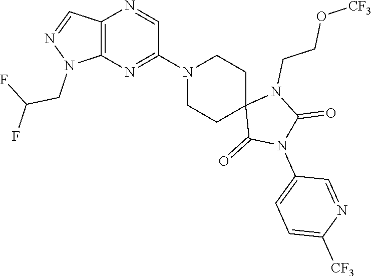 [SMILES: O=C1N(c2ccc(C(F)(F)F)nc2)C(=O)C2(CCN(c3cnc4cnn(CC(F)F)c4n3)CC2)N1CCOC(F)(F)F]

Step 1: tert-butyl 2,4-dioxo-1-(2-(trifluoromethoxy)ethyl)-3-(6-(trifluoromethyl)pyridin-3-yl)-1,3,8-triazaspiro[4.5]decane-8-carboxylate: Followed the general procedure K using tert-butyl 2,4-dioxo-3-(6-(trifluoromethyl)pyridin-3-yl)-1,3,8-triazaspiro[4.5]decane-8-carboxylate (210 mg, 0.507 mmol, 1.0 equiv.) and 1-bromo-2-(trifluoromethoxy)ethane (107 mg, 0.558 mmol, 1.1 equiv.), KI (168 mg, 1.01 mmol, 2 equiv.) as the starting materials to give tert-butyl 2,4-dioxo-1-(2-(trifluoromethoxy)ethyl)-3-(6-(trifluoromethyl)pyridin-3-yl)-1,3,8-triazaspiro[4.5]decane-8-carboxylate (100 mg, 60%) as a colorless oil. MS m/z: 527 [M+H]⁺.

Step 2: 1-(2-(trifluoromethoxy)ethyl)-3-(6-(trifluoromethyl)pyridin-3-yl)-1,3,8-triazaspiro[4.5]decane-2,4-dione hydrochloride: Followed the general procedure B using tert-butyl 2,4-dioxo-1-(2-(trifluoromethoxy)ethyl)-3-(6-(trifluoromethyl)pyridin-3-yl)-1,3,8-triazaspiro[4.5]decane-8-carboxylate (100 mg) as the starting material to give the crude product 1-(2-(trifluoromethoxy)ethyl)-3-(6-(trifluoromethyl)pyridin-3-yl)-1,3,8-triazaspiro[4.5]decane-2,4-dione hydrochloride (80 mg). MS m/z: 427 [M+H]⁺.

Step 3: 8-(1-(2,2-difluoroethyl)-1H-pyrazolo[3,4-b]pyrazin-6-yl)-1-(2-(trifluoromethoxy)ethyl)-3-(6-(trifluoromethyl)pyridin-3-yl)-1,3,8-triazaspiro[4.5]decane-2,4-dione: Followed the general procedure I using 1-(2-(trifluoromethoxy)ethyl)-3-(6-(trifluoromethyl)pyridin-3-yl)-1,3,8-triazaspiro[4.5]decane-2,4-dione hydrochloride (80 mg, 0.187 mmol, 1.0 equiv.) and 6-chloro-1-(2,2-difluoroethyl)-1H-pyrazolo[3,4-b]pyrazine (45 mg, 0.206 mmol, 1.1 equiv.) as the starting materials to give 8-(1-(2,2-difluoroethyl)-1H-pyrazolo[3,4-b]pyrazin-6-yl)-1-(2-(trifluoromethoxy)ethyl)-3-(6-(trifluoromethyl)pyridin-3-yl)-1,3,8-triazaspiro[4.5]decane-2,4-dione (26.8 mg, 48%) as a white solid. ¹H NMR (400 MHz, DMSO-d₆) δ 8.93 (s, 1H), 8.55 (s, 1H), 8.31-8.22 (m, 1H), 8.20-8.10 (m, 2H), 6.45 (tt, J=54.8, 3.8 Hz, 1H), 4.79-4.62 (m, 4H), 4.23 (t, J=5.6 Hz, 2H), 3.74-3.57 (m, 4H), 2.18-2.10 (m, 4H). MS m/z: 609.2 [M+H]⁺.

### Example 878: 1-ethyl-8-(3-methyl-1-(oxetan-3-yl)-1H-pyrazolo[3,4-d]pyrimidin-6-yl)-3-(6-(trifluoromethyl)pyridin-3-yl)-1,3,8-triazaspiro[4.5]decane-2,4-dione

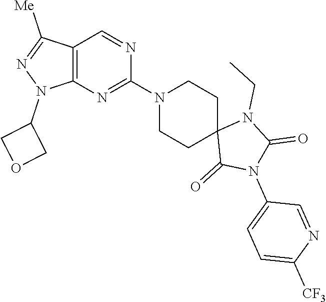 [SMILES: CCN1C(=O)N(c2ccc(C(F)(F)F)nc2)C(=O)C12CCN(c1ncc3c(C)nn(C4COC4)c3n1)CC2]

Step 1: 1-ethyl-8-(3-methyl-1-(oxetan-3-yl)-1H-pyrazolo[3,4-d]pyrimidin-6-yl)-3-(6-(trifluoromethyl)pyridin-3-yl)-1,3,8-triazaspiro[4.5]decane-2,4-dione: Followed the general procedure I using 6-chloro-3-methyl-1-(oxetan-3-yl)-1H-pyrazolo[3,4-d]pyrimidine (50 mg, 0.252 mmol, 1.0 equiv.) and 1-ethyl-3-(6-(trifluoromethyl)pyridin-3-yl)-1,3,8-triazaspiro[4.5]decane-2,4-dione (90 mg, 0.302 mmol, 1.2 equiv.) as the starting materials to give 1-ethyl-8-(3-methyl-1-(oxetan-3-yl)-1H-pyrazolo[3,4-d]pyrimidin-6-yl)-3-(6-(trifluoromethyl)pyridin-3-yl)-1,3,8-triazaspiro[4.5]decane-2,4-dione (71.9 mg, 57%) as a white solid. ¹H NMR (400 MHz, DMSO-d₆) δ 8.95-8.84 (m, 2H), 8.26 (dd, J=8.9, 2.3 Hz, 1H), 8.12 (d, J=8.5 Hz, 1H), 5.86 (p, J=7.2 Hz, 1H), 5.02 (t, J=6.4 Hz, 2H), 4.95 (t, J=7.1 Hz, 2H), 4.89 (d, J=13.2 Hz, 2H), 3.56 (t, J=12.4 Hz, 2H), 3.32-3.26 (m, 2H), 2.49 (s, 3H), 2.15-2.08 (m, 2H), 2.04-1.96 (m, 2H), 1.15 (t, J=7.0 Hz, 3H). MS m/z: 531.2 [M+H]⁺.

### Example 879: 8-(1-(2,2-difluoroethyl)-1H-pyrazolo[3,4-b]pyrazin-6-yl)-1-methyl-3-(6-(trifluoromethyl)pyridin-3-yl)-1,3,8-triazaspiro[4.5]decane-2,4-dione

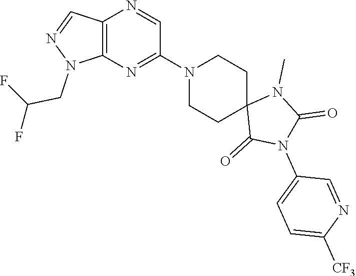

Step 1: tert-butyl 1-methyl-2,4-dioxo-3-(6-(trifluoromethyl)pyridin-3-yl)-1,3,8-triazaspiro[4.5]decane-8-carboxylate: Followed the general procedure K using tert-butyl 2,4-dioxo-3-(6-(trifluoromethyl)pyridin-3-yl)-1,3,8-triazaspiro[4.5]decane-8-carboxylate (150 mg, 0.362 mmol, 1.0 equiv.) and Mel (61.7 mg, 0.434 mmol, 1.2 equiv.) as the starting materials to give tert-butyl 1-methyl-2,4-dioxo-3-(6-(trifluoromethyl)pyridin-3-yl)-1,3,8-triazaspiro[4.5]decane-8-carboxylate (130 mg, 83.3%) as a light yellow solid. MS m/z: 429 [M+H]⁺.

Step 2: 1-methyl-3-(6-(trifluoromethyl)pyridin-3-yl)-1,3,8-triazaspiro[4.5]decane-2,4-dione hydrochloride: Followed the general procedure B using tert-butyl 1-methyl-2,4-dioxo-3-(6-(trifluoromethyl)pyridin-3-yl)-1,3,8-triazaspiro[4.5]decane-8-carboxylatea (130 mg, 0.303 mmol, 1.0 equiv.) as the starting material to give the crude product 1-methyl-3-(6-(trifluoromethyl)pyridin-3-yl)-1,3,8-triazaspiro[4.5]decane-2,4-dione hydrochloride (110 mg). MS m/z: 329 [M+H]⁺.

Step 3: 8-(1-(2,2-difluoroethyl)-1H-pyrazolo[3,4-b]pyrazin-6-yl)-1-methyl-3-(6-(trifluoromethyl)pyridin-3-yl)-1,3,8-triazaspiro[4.5]decane-2,4-dione: Followed the general procedure I using 1-methyl-3-(6-(trifluoromethyl)pyridin-3-yl)-1,3,8-triazaspiro[4.5]decane-2,4-dione hydrochloride (100 mg, 0.274 mmol, 1.0 equiv.) and 6-chloro-1-(2,2-difluoroethyl)-1H-pyrazolo[3,4-b]pyrazine (59.9 mg, 0.274 mmol, 1.0 equiv.) as the starting materials to give 8-(1-(2,2-difluoroethyl)-1H-pyrazolo[3,4-b]pyrazin-6-yl)-1-methyl-3-(6-(trifluoromethyl)pyridin-3-yl)-1,3,8-triazaspiro[4.5]decane-2,4-dione (85.4 mg, 60.6%) as a white solid. ¹H NMR (400 MHz, DMSO-d₆) δ 8.92 (d, 1H), 8.55 (s, 1H), 8.25 (dd, 1H), 8.16 (s, 1H), 8.12 (d, 1H), 6.46 (tt, 1H), 4.88-4.46 (m, 4H), 3.64 (dt, 2H), 2.86 (s, 3H), 2.25-2.05 (m, 4H). MS m/z: 511.2 [M+H]⁺.

### Example 880: 1-(2-cyclopropylethyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione

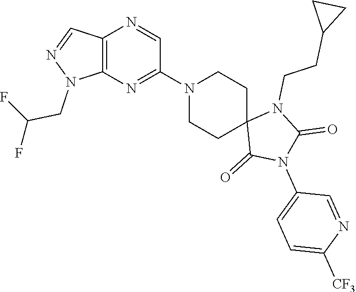

Step 1: tert-butyl 1-(2-cyclopropylethyl)-2,4-dioxo-3-(6-(trifluoromethyl)pyridin-3-yl)-1,3,8-triazaspiro[4.5]decane-8-carboxylate: Followed the general procedure I using tert-butyl 2,4-dioxo-3-(6-(trifluoromethyl)pyridin-3-yl)-1,3,8-triazaspiro[4.5]decane-8-carboxylate (150 mg, 361 μmol, 1.0 equiv.) and (2-bromoethyl)cyclopropane (60 mg, 397 μmol, 1.1 equiv.) as the starting materials to give tert-butyl 1-(2-cyclopropylethyl)-2,4-dioxo-3-(6-(trifluoromethyl)pyridin-3-yl)-1,3,8-triazaspiro[4.5]decane-8-carboxylate (100 mg, 57%) as a white solid. MS m/z: 483 [M+H]⁺.

Step 2: 1-(2-cyclopropylethyl)-3-(6-(trifluoromethyl)pyridin-3-yl)-1,3,8-triazaspiro[4.5]decane-2,4-dione: Followed the general procedure H using tert-butyl 1-(2-cyclopropylethyl)-2,4-dioxo-3-(6-(trifluoromethyl)pyridin-3-yl)-1,3,8-triazaspiro[4.5]decane-8-carboxylate (100 mg) as the starting material to give the crude product 1-(2-cyclopropylethyl)-3-(6-(trifluoromethyl)pyridin-3-yl)-1,3,8-triazaspiro[4.5]decane-2,4-dione (80 mg). MS m/z: 383 [M+H]⁺.

Step 3: 1-(2-cyclopropylethyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione: Followed the general procedure I using 1-(2-cyclopropylethyl)-3-(6-(trifluoromethyl)pyridin-3-yl)-1,3,8-triazaspiro[4.5]decane-2,4-dione (80 mg, 208 μmol, 1.0 equiv.) and 6-chloro-1-(2,2-difluoroethyl)-1H-pyrazolo[3,4-b]pyrazine (50 mg, 229 μmol, 1.1 equiv.) as the starting materials to give 1-(2-cyclopropylethyl)-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione (59 mg, 50%) as a white solid. ¹H NMR (400 MHz, DMSO-d₆) δ 8.92 (d, J=2.3 Hz, 1H), 8.54 (s, 1H), 8.28-8.21 (m, 1H), 8.18-8.08 (m, 2H), 6.58-6.33 (m, 1H), 4.78-4.61 (m, 4H), 3.68-3.56 (m, 2H), 3.34 (s, 2H), 2.11 (d, J=31.6 Hz, 5H), 1.51 (q, J=7.4 Hz, 2H), 0.76-0.64 (m, 1H), 0.44-0.35 (m, 2H), 0.09-0.03 (m, 2H). MS m/z: 565.00 [M+H]⁺.

### Example 881: 8-(1-(2,2-difluoroethyl)-1H-pyrazolo[3,4-b]pyrazin-6-yl)-1-(4,4,4-trifluorobutyl)-3-(6-(trifluoromethyl)pyridin-3-yl)-1,3,8-triazaspiro[4.5]decane-2,4-dione

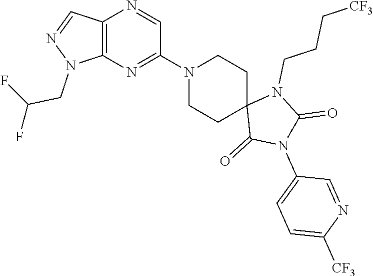

Step 1: tert-butyl 2,4-dioxo-1-(4,4,4-trifluorobutyl)-3-(6-(trifluoromethyl)pyridin-3-yl)-1,3,8-triazaspiro[4.5]decane-8-carboxylate: Followed the general procedure I using tert-butyl 2,4-dioxo-3-(6-(trifluoromethyl)pyridin-3-yl)-1,3,8-triazaspiro[4.5]decane-8-carboxylate (150 mg, 361 μmol, 1.0 equiv.) and 4-bromo-1,1,1-trifluorobutane (76 mg, 397 μmol, 1.1 equiv.) as the starting materials to give tert-butyl 2,4-dioxo-1-(4,4,4-trifluorobutyl)-3-(6-(trifluoromethyl)pyridin-3-yl)-1,3,8-triazaspiro[4.5]decane-8-carboxylate (130 mg, 68%) as a white solid. MS m/z: 525 [M+H]⁺.

Step 2: 1-(4,4,4-trifluorobutyl)-3-(6-(trifluoromethyl)pyridin-3-yl)-1,3,8-triazaspiro[4.5]decane-2,4-dione: Followed the general procedure H using tert-butyl 2,4-dioxo-1-(4,4,4-trifluorobutyl)-3-(6-(trifluoromethyl)pyridin-3-yl)-1,3,8-triazaspiro[4.5]decane-8-carboxylate (130 mg) as the starting material to give the crude product 1-(4,4,4-trifluorobutyl)-3-(6-(trifluoromethyl)pyridin-3-yl)-1,3,8-triazaspiro[4.5]decane-2,4-dione (100 mg). MS m/z: 425 [M+H]⁺.

Step 3: 8-(1-(2,2-difluoroethyl)-1H-pyrazolo[3,4-b]pyrazin-6-yl)-1-(4,4,4-trifluorobutyl)-3-(6-(trifluoromethyl)pyridin-3-yl)-1,3,8-triazaspiro[4.5]decane-2,4-dione: Followed the general procedure I using 1-(4,4,4-trifluorobutyl)-3-(6-(trifluoromethyl)pyridin-3-yl)-1,3,8-triazaspiro[4.5]decane-2,4-dione (80 mg, 188 μmol, 1.0 equiv.) and 6-chloro-1-(2,2-difluoroethyl)-1H-pyrazolo[3,4-b]pyrazine (46 mg, 207 μmol, 1.1 equiv.) as the starting materials to give 8-(1-(2,2-difluoroethyl)-1H-pyrazolo[3,4-b]pyrazin-6-yl)-1-(4,4,4-trifluorobutyl)-3-(6-(trifluoromethyl)pyridin-3-yl)-1,3,8-triazaspiro[4.5]decane-2,4-dione (99 mg, 86%) as a white solid. ¹H NMR (400 MHz, DMSO-d₆) δ 8.93 (d, J=2.3 Hz, 1H), 8.55 (s, 1H), 8.26 (dd, J=8.4, 2.4 Hz, 1H), 8.19-8.09 (m, 2H), 6.45 (tt, J=54.9, 3.8 Hz, 1H), 4.77-4.64 (m, 4H), 3.66-3.59 (m, 2H), 3.37 (t, J=7.5 Hz, 2H), 2.38-2.30 (m, 2H), 2.20 (d, J=12.9 Hz, 2H), 2.09 (d, J=12.3 Hz, 3H), 1.88-1.75 (m, 2H). MS m/z: 607.00 [M+H]⁺.

### Example 882: 1-cyclobutyl-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione

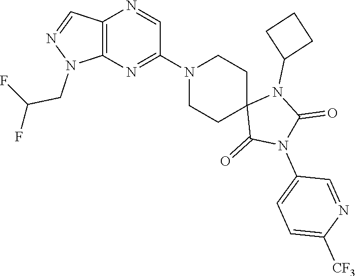

Step 1: tert-butyl 1-cyclobutyl-2,4-dioxo-3-(6-(trifluoromethyl)pyridin-3-yl)-1,3,8-triazaspiro[4.5]decane-8-carboxylate: Followed the general procedure K using tert-butyl 2,4-dioxo-3-(2-(trifluoromethyl)pyridin-4-yl)-1,3,8-triazaspiro[4.5]decane-8-carboxylate (210 mg, 0.507 mmol, 1.0 equiv.) and bromocyclobutane (81.5 mg, 0.608 mmol, 1.2 equiv.) as the starting materials to give tert-butyl 1-cyclobutyl-2,4-dioxo-3-(6-(trifluoromethyl)pyridin-3-yl)-1,3,8-triazaspiro[4.5]decane-8-carboxylate (60 mg, 32%) as a colorless oil. MS m/z: 469[M+H]⁺.

Step 2: 1-cyclobutyl-3-(6-(trifluoromethyl)pyridin-3-yl)-1,3,8-triazaspiro[4.5]decane-2,4-dione hydrochloride: Followed the general procedure B using tert-butyl 1-cyclobutyl-2,4-dioxo-3-(6-(trifluoromethyl)pyridin-3-yl)-1,3,8-triazaspiro[4.5]decane-8-carboxylate (60 mg) as the starting material to give the crude product 1-cyclobutyl-3-(6-(trifluoromethyl)pyridin-3-yl)-1,3,8-triazaspiro[4.5]decane-2,4-dione hydrochloride (50 mg). MS m/z: 369 [M+H]⁺.

Step 3: 1-cyclobutyl-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione: Followed the general procedure I using 1-cyclobutyl-3-(6-(trifluoromethyl)pyridin-3-yl)-1,3,8-triazaspiro[4.5]decane-2,4-dione hydrochloride (50 mg, 0.135 mmol, 1.0 equiv.) and 6-chloro-1-(2,2-difluoroethyl)-1H-pyrazolo[3,4-b]pyrazine (35.4 mg, 0.162 mmol, 1.2 equiv.) as the starting materials to give 1-cyclobutyl-8-(1-(2,2-difluoroethyl)-1H-pyrazolo[3,4-b]pyrazin-6-yl)-3-(6-(trifluoromethyl)pyridin-3-yl)-1,3,8-triazaspiro[4.5]decane-2,4-dione (16.4 mg, 49%) as a white solid. ¹H NMR (400 MHz, DMSO-d₆) δ 8.94 (s, 1H), 8.55 (s, 1H), 8.28 (dd, J=8.4, 2.4 Hz, 1H), 8.20-8.07 (m, 2H), 6.46 (tt, J=54.8, 3.8 Hz, 1H), 4.81-4.57 (m, 4H), 3.71-3.58 (m, 2H), 3.22 (d, J=6.8 Hz, 2H), 2.26-2.11 (m, 4H), 1.14-0.98 (m, 1H), 0.51-0.38 (m, 2H), 0.34-0.20 (m, 2H). MS m/z: 551.2 [M+H]⁺.

### Example 883: 8-(1-(2,2-difluoroethyl)-1H-pyrazolo[3,4-b]pyrazin-6-yl)-1-((5-fluoropyridin-3-yl)methyl)-3-(6-(trifluoromethyl)pyridin-3-yl)-1,3,8-triazaspiro[4.5]decane-2,4-dione

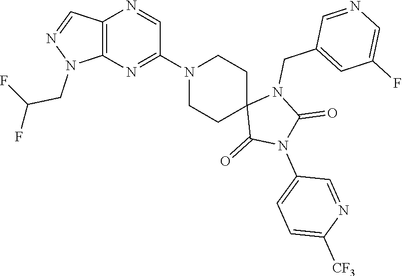

Step 1: tert-butyl 1-((5-fluoropyridin-3-yl)methyl)-2,4-dioxo-3-(6-(trifluoromethyl)pyridin-3-yl)-1,3,8-triazaspiro[4.5]decane-8-carboxylate: Followed the general procedure K using tert-butyl 2,4-dioxo-3-(6-(trifluoromethyl)pyridin-3-yl)-1,3,8-triazaspiro[4.5]decane-8-carboxylate (160 mg, 0.386 mmol, 1.0 equiv.) and 3-(chloromethyl)-5-fluoropyridine (61.6 mg, 0.425 mmol, 1.1 equiv.) as the starting materials to give tert-butyl 1-((5-fluoropyridin-3-yl)methyl)-2,4-dioxo-3-(6-(trifluoromethyl)pyridin-3-yl)-1,3,8-triazaspiro[4.5]decane-8-carboxylate (110 mg, 87%) as a colorless oil. MS m/z: [M+H]⁺.

Step 2: 1-((5-fluoropyridin-3-yl)methyl)-3-(6-(trifluoromethyl)pyridin-3-yl)-1,3,8-triazaspiro[4.5]decane-2,4-dione hydrochloride: Followed the general procedure B using tert-butyl 1-((5-fluoropyridin-3-yl)methyl)-2,4-dioxo-3-(6-(trifluoromethyl)pyridin-3-yl)-1,3,8-triazaspiro[4.5]decane-8-carboxylate (110 mg) as the starting material to give the crude product 1-((5-fluoropyridin-3-yl)methyl)-3-(6-(trifluoromethyl)pyridin-3-yl)-1,3,8-triazaspiro[4.5]decane-2,4-dione hydrochloride (80 mg). MS m/z: 424[M+H]⁺.

Step 3: 8-(1-(2,2-difluoroethyl)-1H-pyrazolo[3,4-b]pyrazin-6-yl)-1-((5-fluoropyridin-3-yl)methyl)-3-(6-(trifluoromethyl)pyridin-3-yl)-1,3,8-triazaspiro[4.5]decane-2,4-dione: Followed the general procedure I using 1-((5-fluoropyridin-3-yl)methyl)-3-(6-(trifluoromethyl)pyridin-3-yl)-1,3,8-triazaspiro[4.5]decane-2,4-dione hydrochloride (80 mg, 0.189 mmol, 1.0 equiv.) and 6-chloro-1-(2,2-difluoroethyl)-1H-pyrazolo[3,4-b]pyrazine (45.3 mg, 0.208 mmol, 1.1 equiv.) as the starting materials to give 8-(1-(2,2-difluoroethyl)-1H-pyrazolo[3,4-b]pyrazin-6-yl)-1-((5-fluoropyridin-3-yl)methyl)-3-(6-(trifluoromethyl)pyridin-3-yl)-1,3,8-triazaspiro[4.5]decane-2,4-dione (61.2 mg, 53%) as a white solid. ¹H NMR (400 MHz, DMSO-d₆) δ 8.98 (d, J=2.4 Hz, 1H), 8.55-8.45 (m, 3H), 8.32 (dd, J=8.4, 2.4 Hz, 1H), 8.20-8.09 (m, 2H), 7.83-7.74 (m, 1H), 6.43 (tt, J=54.8, 3.8 Hz, 1H), 4.77-4.59 (m, 6H), 3.61 (t, J=12.8 Hz, 2H), 2.28-2.17 (m, 2H), 2.11-2.05 (m, 2H). MS m/z: 606.3 [M+H]⁺.

### Example 884: 8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decan-1-one

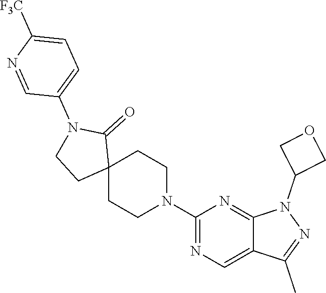 [SMILES: Cc1nn(C2COC2)c2nc(N3CCC4(CC3)CCN(c3ccc(C(F)(F)F)nc3)C4=O)ncc12]

Step 1: 8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decan-1-one: Followed the general procedure I using 2-(6-(trifluoromethyl)pyridin-3-yl)-2,8-diazaspiro[4.5]decan-1-one (100 mg, 0.334 mmol, 1.0 equiv.) and 6-chloro-3-methyl-1-(oxetan-3-yl)-1H-pyrazolo[3,4-d]pyrimidine (74.9 mg, 0.158 mmol, 1.0 equiv.) as the starting materials to give 8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decan-1-one (64.1 mg, 39%) as a white solid. ¹H NMR (400 MHz, DMSO-d₆) δ 8.88 (d, J=1.8 Hz, 1H), 8.81 (s, 1H), 8.52 (d, J=8.9 Hz, 1H), 8.25-8.18 (m, 1H), 5.89-5.77 (m, 1H), 5.01 (t, J=6.4 Hz, 2H), 4.94 (t, J=7.1 Hz, 2H), 4.64 (d, J=13.3 Hz, 2H), 4.06 (t, J=7.0 Hz, 2H), 3.31 (s, 2H), 2.48 (s, 3H), 2.21 (t, J=7.0 Hz, 2H), 1.81-1.62 (m, 4H). MS m/z: 488.3 [M+H]⁺.

### Example 885: 8-(3-methyl-1-(oxetan-3-yl)-1H-pyrazolo[3,4-b]pyrazin-6-yl)-2-(6-(trifluoromethyl)pyridin-3-yl)-2,8-diazaspiro[4.5]decan-1-one

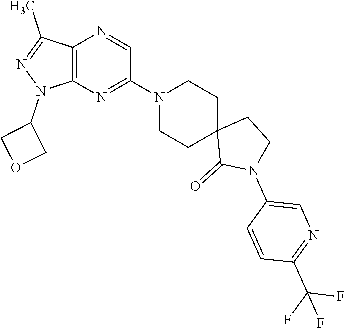 [SMILES: Cc1nn(C2COC2)c2nc(N3CCC4(CC3)CCN(c3ccc(C(F)(F)F)nc3)C4=O)cnc12]

Step 1: 6-chloro-3-methyl-1-(oxetan-3-yl)-1H-pyrazolo[3,4-b]pyrazine: Followed the general procedure K using 6-chloro-3-methyl-1H-pyrazolo[3,4-b]pyrazine (300 mg, 1.79 mmol, 1.0 equiv.) and oxetan-3-yl trifluoromethanesulfonate (552 mg, 2.69 mmol, 1.5 equiv.) as the starting materials to give 6-chloro-3-methyl-1-(oxetan-3-yl)-1H-pyrazolo[3,4-b]pyrazine (300 mg, 75%) as a white solid. MS m/z: 225 [M+H]⁺.

Step 2: 8-(3-methyl-1-(oxetan-3-yl)-1H-pyrazolo[3,4-b]pyrazin-6-yl)-2-(6-(trifluoromethyl)pyridin-3-yl)-2,8-diazaspiro[4.5]decan-1-one: Followed the general procedure I using 6-chloro-3-methyl-1-(oxetan-3-yl)-1H-pyrazolo[3,4-b]pyrazine (60 mg, 268 μmol, 1.0 equiv.) and 2-(6-(trifluoromethyl)pyridin-3-yl)-2,8-diazaspiro[4.5]decan-1-one hydrochloride (143 mg, 402 μmol, 1.5 equiv.) as the starting materials to give 8-(3-methyl-1-(oxetan-3-yl)-1H-pyrazolo[3,4-b]pyrazin-6-yl)-2-(6-(trifluoromethyl)pyridin-3-yl)-2,8-diazaspiro[4.5]decan-1-one (87.1 mg, 67%) as a white solid. ¹H NMR (400 MHz, DMSO-d₆) δ 9.09 (d, J=2.5 Hz, 1H), 8.46-8.38 (m, 2H), 7.94 (d, J=8.7 Hz, 1H), 5.84 (p, J=7.2 Hz, 1H), 5.08-5.01 (m, 2H), 4.98-4.90 (m, 2H), 4.45-4.35 (m, 2H), 3.98 (t, J=6.9 Hz, 2H), 3.40-3.36 (m, 1H), 3.32-3.29 (m, 1H), 2.47 (s, 3H), 2.26 (t, J=6.9 Hz, 2H), 1.88-1.76 (m, 2H), 1.76-1.66 (m, 2H). MS m/z: 488.05 [M+H]⁺.

### Example 886: 8-(1-(oxetan-3-yl)-1H-pyrazolo[3,4-b]pyrazin-6-yl)-2-(6-(trifluoromethyl)pyridin-3-yl)-2,8-diazaspiro[4.5]decan-1-one

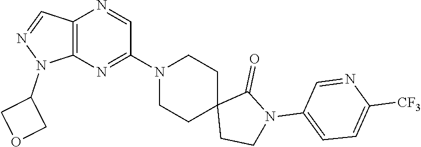 [SMILES: O=C1N(c2ccc(C(F)(F)F)nc2)CCC12CCN(c1cnc3cnn(C4COC4)c3n1)CC2]

Step 1: tert-butyl 1-oxo-2-(6-(trifluoromethyl)pyridin-3-yl)-2,8-diazaspiro[4.5]decane-8-carboxylate: Followed the general procedure I using tert-butyl 1-oxo-2,8-diazaspiro[4.5]decane-8-carboxylate (150 mg, 588 μmol, 1.0 equiv.) and 5-fluoro-2-(trifluoromethyl)pyridine (108 mg, 647 μmol, 1.1 equiv.) as the starting materials to give tert-butyl 1-oxo-2-(6-(trifluoromethyl)pyridin-3-yl)-2,8-diazaspiro[4.5]decane-8-carboxylate (110 mg, 46%) as a white solid. MS m/z: 400 [M+H]⁺.

Step 2: 2-(6-(trifluoromethyl)pyridin-3-yl)-2,8-diazaspiro[4.5]decan-1-one: Followed the general procedure H using tert-butyl 1-oxo-2-(6-(trifluoromethyl)pyridin-3-yl)-2,8-diazaspiro[4.5]decane-8-carboxylate (110 mg) as the starting material to give the crude product 2-(6-(trifluoromethyl)pyridin-3-yl)-2,8-diazaspiro[4.5]decan-1-one (80 mg). MS m/z: 300 [M+H]⁺.

Step 3: 8-(1-(oxetan-3-yl)-1H-pyrazolo[3,4-b]pyrazin-6-yl)-2-(6-(trifluoromethyl)pyridin-3-yl)-2,8-diazaspiro[4.5]decan-1-one: Followed the general procedure I using 2-(6-(trifluoromethyl)pyridin-3-yl)-2,8-diazaspiro[4.5]decan-1-one (80 mg, 266 μmol, 1.0 equiv.) and 6-chloro-1-(oxetan-3-yl)-1H-pyrazolo[3,4-b]pyrazine (62 mg, 293 μmol, 1.1 equiv.) as the starting materials to give 8-(1-(oxetan-3-yl)-1H-pyrazolo[3,4-b]pyrazin-6-yl)-2-(6-(trifluoromethyl)pyridin-3-yl)-2,8-diazaspiro[4.5]decan-1-one (77 mg, 61%) as a white solid. ¹H NMR (400 MHz, DMSO-d₆) δ 9.09 (d, J=2.5 Hz, 1H), 8.51 (s, 1H), 8.42 (dd, J=8.7, 2.5 Hz, 1H), 8.20 (s, 1H), 7.94 (d, J=8.7 Hz, 1H), 5.91-5.63 (m, J=7.1 Hz, 1H), 5.06 (t, J=6.4 Hz, 2H), 4.97 (dd, J=7.8, 6.4 Hz, 2H), 4.42 (dt, J=13.7, 4.2 Hz, 2H), 3.98 (t, J=6.9 Hz, 2H), 3.41-3.33 (m, 2H), 2.26 (t, J=6.9 Hz, 2H), 1.83 (ddd, J=15.0, 11.4, 4.2 Hz, 2H), 1.71 (dt, J=13.4, 3.7 Hz, 2H). MS m/z: 474.05 [M+H]⁺.

### Example 887: 8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decane

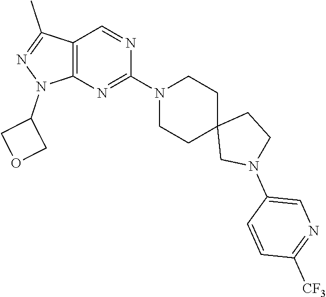 [SMILES: Cc1nn(C2COC2)c2nc(N3CCC4(CCN(c5ccc(C(F)(F)F)nc5)C4)CC3)ncc12]

8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decane: Followed the general procedure I using 2-(6-(trifluoromethyl)pyridin-3-yl)-2,8-diazaspiro[4.5]decane hydrochloride (80 mg, 0.28 mmol, 1.0 equiv.) and 6-chloro-3-methyl-1-(oxetan-3-yl)-1H-pyrazolo[3,4-d]pyrimidine (63 mg, 0.28 mmol, 1.0 equiv.) as the starting materials to give 8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decane (72.3 mg, 35%) as a white solid. ¹H NMR (400 MHz, DMSO-d₆) δ 8.86 (s, 1H), 8.04 (d, J=2.7 Hz, 1H), 7.59 (d, J=8.7 Hz, 1H), 7.00 (dd, J=8.8, 2.8 Hz, 1H), 6.03-5.60 (m, 1H), 5.20-4.80 (m, 4H), 4.14-3.90 (m, 2H), 3.83 (s, 2H), 3.45 (t, J=7.0 Hz, 4H), 2.47 (s, 3H), 1.96 (t, J=6.9 Hz, 2H), 1.78-1.41 (m, 4H). MS m/z: 474.25 [M+H]⁺.

### Example 888: 8-(3-methyl-1-(oxetan-3-yl)-1H-pyrazolo[3,4-b]pyrazin-6-yl)-2-(6-(trifluoromethyl)pyridin-3-yl)-2,8-diazaspiro[4.5]decane

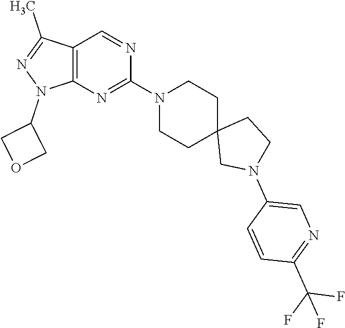 [SMILES: Cc1nn(C2COC2)c2nc(N3CCC4(CCN(c5ccc(C(F)(F)F)nc5)C4)CC3)ncc12]

Step 1: 8-(3-methyl-1-(oxetan-3-yl)-1H-pyrazolo[3,4-b]pyrazin-6-yl)-2-(6-(trifluoromethyl)pyridin-3-yl)-2,8-diazaspiro[4.5]decane: Followed the general procedure I using 6-chloro-3-methyl-1-(oxetan-3-yl)-1H-pyrazolo[3,4-b]pyrazine (60 mg, 268 μmol, 1.0 equiv.) and 2-(6-(trifluoromethyl)pyridin-3-yl)-2,8-diazaspiro[4.5]decane hydrochloride (137 mg, 402 μmol, 1.5 equiv.) as the starting materials to give 8-(3-methyl-1-(oxetan-3-yl)-1H-pyrazolo[3,4-b]pyrazin-6-yl)-2-(6-(trifluoromethyl)pyridin-3-yl)-2,8-diazaspiro[4.5]decane (106.3 mg, 84%) as a white solid. ¹H NMR (400 MHz, DMSO-d₆) δ 8.40 (s, 1H), 8.04 (d, J=2.7 Hz, 1H), 7.59 (d, J=8.7 Hz, 1H), 7.00 (dd, J=8.8, 2.8 Hz, 1H), 5.83 (p, J=7.2 Hz, 1H), 5.08-5.00 (m, 2H), 4.98-4.90 (m, 2H), 3.92-3.81 (m, 2H), 3.78-3.69 (m, 2H), 3.45 (t, J=7.0 Hz, 2H), 2.46 (s, 3H), 1.97 (t, J=7.0 Hz, 2H), 1.74-1.59 (m, 4H). MS m/z: 474.05 [M+H]⁺.

### Example 889: 8-(1-ethyl-1H-pyrazolo[3,4-d]pyrimidin-6-yl)-2-(6-(trifluoromethyl)pyridin-3-yl)-2,8-diazaspiro[4.5]decan-3-one

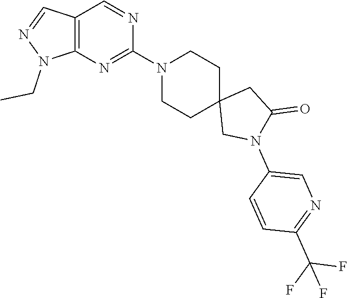 [SMILES: CCn1ncc2cnc(N3CCC4(CC3)CC(=O)N(c3ccc(C(F)(F)F)nc3)C4)nc21]

Step 1: 6-chloro-1-ethyl-1H-pyrazolo[3,4-d]pyrimidine: Followed the general procedure K using 6-chloro-1H-pyrazolo[3,4-d]pyrimidine (100 mg, 0.645 mmol, 1.0 equiv.) and EtI (110 mg, 0.710 mmol, 1.1 equiv.) as the starting materials to give 6-chloro-1-ethyl-1H-pyrazolo[3,4-d]pyrimidine (80.0 mg, 67.8%) as a light white solid. MS m/z: 183 [M+H]⁺.

Step 2: 8-(1-ethyl-1H-pyrazolo[3,4-d]pyrimidin-6-yl)-2-(6-(trifluoromethyl)pyridin-3-yl)-2,8-diazaspiro[4.5]decan-3-one: Followed the general procedure I using 6-chloro-1-ethyl-1H-pyrazolo[3,4-d]pyrimidine (80.0 mg, 0.523 mmol, 1.0 equiv.) and 2-(6-(trifluoromethyl)pyridin-3-yl)-2,8-diazaspiro[4.5]decan-3-one hydrochloride (157 mg, 0.523 mmol, 1.0 equiv.) as the starting materials to give 8-(1-ethyl-1H-pyrazolo[3,4-d]pyrimidin-6-yl)-2-(6-(trifluoromethyl)pyridin-3-yl)-2,8-diazaspiro[4.5]decan-3-one (31.6 mg, 16.2%) as a white solid. ¹H NMR (400 MHz, DMSO-d₆) δ 9.05 (d, 1H), 8.88 (s, 1H), 8.40 (dd, 1H), 7.98 (s, 1H), 7.94 (d, 1H), 4.25 (q, 2H), 4.03 (dt, 5.3 Hz, 2H), 3.94-3.77 (m, 4H), 2.63 (s, 2H), 1.70 (t, 4H), 1.37 (t, 3H). MS m/z: 446.25 [M+H]⁺.

### Example 890: 8-(1-ethyl-1H-pyrazolo[3,4-b]pyrazin-6-yl)-2-(6-(trifluoromethyl)pyridin-3-yl)-2,8-diazaspiro[4.5]decan-3-one

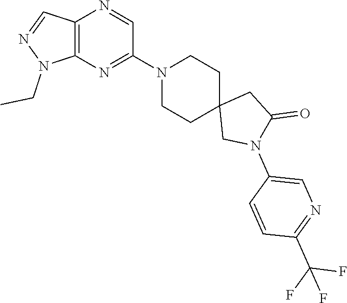 [SMILES: CCn1ncc2ncc(N3CCC4(CC3)CC(=O)N(c3ccc(C(F)(F)F)nc3)C4)nc21]

Step 1: 6-chloro-1-ethyl-1H-pyrazolo[3,4-b]pyrazine: Followed the general procedure K using 6-chloro-1H-pyrazolo[3,4-b]pyrazine (100 mg, 0.645 mmol, 1.0 equiv.) and EtI (110 mg, 0.710 mmol, 1.1 equiv.) as the starting materials to give 6-chloro-1-ethyl-1H-pyrazolo[3,4-b]pyrazine (80.0 mg, 67.8%) as a white solid. MS m/z: 183 [M+H]⁺.

Step 2: 8-(1-ethyl-1H-pyrazolo[3,4-b]pyrazin-6-yl)-2-(6-(trifluoromethyl)pyridin-3-yl)-2,8-diazaspiro[4.5]decan-3-one: Followed the general procedure I using 6-chloro-1-ethyl-1H-pyrazolo[3,4-b]pyrazine (80.0 mg, 0.523 mmol, 1.0 equiv.) and 2-(6-(trifluoromethyl)pyridin-3-yl)-2,8-diazaspiro[4.5]decan-3-one hydrochloride (157 mg, 0.523 mmol, 1.0 equiv.) as the starting materials to give 8-(1-ethyl-1H-pyrazolo[3,4-b]pyrazin-6-yl)-2-(6-(trifluoromethyl)pyridin-3-yl)-2,8-diazaspiro[4.5]decan-3-one (61.8 mg, 31.7%) as a white solid. ¹H NMR (400 MHz, DMSO-d₆) δ 9.05 (d, 1H), 8.47 (s, 1H), 8.40 (dd, 1H), 8.04 (s, 1H), 7.95 (d, 1H), 4.28 (q, 2H), 3.95-3.85 (m, 4H), 3.75 (dt, 2H), 2.64 (s, 2H), 1.82-1.70 (m, 4H), 1.39 (t, 3H). MS m/z: 446.3 [M+H]⁺.

### Example 891: 2-(6-chloropyridin-3-yl)-8-(1-(2,2-difluoroethyl)-1H-pyrazolo[3,4-b]pyrazin-6-yl)-2,8-diazaspiro[4.5]decan-3-one

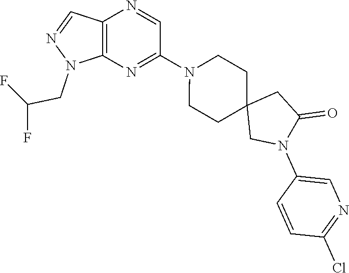 [SMILES: O=C1CC2(CCN(c3cnc4cnn(CC(F)F)c4n3)CC2)CN1c1ccc(Cl)nc1]

Step 1: tert-butyl 2-(6-chloropyridin-3-yl)-3-oxo-2,8-diazaspiro[4.5]decane-8-carboxylate: Followed the general procedure Y using tert-butyl 3-oxo-2,8-diazaspiro[4.5]decane-8-carboxylate (200 mg, 787 μmol, 1.0 equiv.) and 2-chloro-5-iodopyridine (376 mg, 1.57 mmol, 2.0 equiv.) as the starting materials, Ephos Pd G4 (72 mg, 78 umol, 0.1 equiv.) as the catalyst to give tert-butyl 2-(6-chloropyridin-3-yl)-3-oxo-2,8-diazaspiro[4.5]decane-8-carboxylate (60 mg, 20%) as a colorless oil. MS m/z: 366 [M+H]⁺.

Step 2: 2-(6-chloropyridin-3-yl)-2,8-diazaspiro[4.5]decan-3-one hydrochloride: Followed the general procedure B using tert-butyl 2-(6-chloropyridin-3-yl)-3-oxo-2,8-diazaspiro[4.5]decane-8-carboxylate (60 mg) as the starting material to give the crude product 2-(6-chloropyridin-3-yl)-2,8-diazaspiro[4.5]decan-3-one hydrochloride (40 mg). MS m/z: 266 [M+H]⁺.

Step 3: 2-(6-chloropyridin-3-yl)-8-(1-(2,2-difluoroethyl)-1H-pyrazolo[3,4-b]pyrazin-6-yl)-2,8-diazaspiro[4.5]decan-3-one: Followed the general procedure I using 2-(6-chloropyridin-3-yl)-2,8-diazaspiro[4.5]decan-3-one hydrochloride (40 mg, 132 μmol, 1.0 equiv.) and 6-chloro-1-(2,2-difluoroethyl)-1H-pyrazolo[3,4-b]pyrazine (35 mg, 159 μmol, 1.2 equiv.) as the starting materials to give 2-(6-chloropyridin-3-yl)-8-(1-(2,2-difluoroethyl)-1H-pyrazolo[3,4-b]pyrazin-6-yl)-2,8-diazaspiro[4.5]decan-3-one (22 mg, 37%) as a white solid. ¹H NMR (400 MHz, DMSO-d₆) δ 8.68 (d, J=2.8 Hz, 1H), 8.51 (s, 1H), 8.25 (dd, J=8.8, 2.9 Hz, 1H), 8.14 (s, 1H), 7.55 (d, J=8.8 Hz, 1H), 6.44 (tt, J=54.9, 3.8 Hz, 1H), 4.70 (td, J=15.1, 3.8 Hz, 2H), 3.91 (dt, J=13.9, 5.3 Hz, 2H), 3.86-3.71 (m, 4H), 2.59 (s, 2H), 1.81-1.68 (m, 4H). MS m/z: 448.2 [M+H]⁺.

### Example 892: 2-(6-chloropyridin-3-yl)-8-(1-(2,2-difluoroethyl)-1H-pyrazolo[3,4-b]pyrazin-6-yl)-2,8-diazaspiro[4.5]decan-3-one

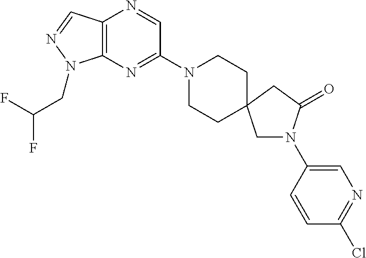 [SMILES: O=C1CC2(CCN(c3cnc4cnn(CC(F)F)c4n3)CC2)CN1c1ccc(Cl)nc1]

Step 1: tert-butyl 2-(5-chloropyridin-2-yl)-3-oxo-2,8-diazaspiro[4.5]decane-8-carboxylate: Followed the general procedure I using tert-butyl 3-oxo-2,8-diazaspiro[4.5]decane-8-carboxylate (120 mg, 0.47 mmol, 1.0 equiv.) and 5-chloro-2-fluoropyridine (80 mg, 0.61 mmol, 1.3 equiv.) as the starting materials to give tert-butyl 2-(5-chloropyridin-2-yl)-3-oxo-2,8-diazaspiro[4.5]decane-8-carboxylate (120 mg, 69%) as a white solid. MS m/z: 366 [M+H]⁺.

Step 2: 2-(5-chloropyridin-2-yl)-2,8-diazaspiro[4.5]decan-3-one hydrochloride: Followed the general procedure B using tert-butyl 2-(5-chloropyridin-2-yl)-3-oxo-2,8-diazaspiro[4.5]decane-8-carboxylate (120 mg, 0.327 mmol, 1.0 equiv.) as the starting material to give the crude product 2-(5-chloropyridin-2-yl)-2,8-diazaspiro[4.5]decan-3-one hydrochloride (120 mg). MS m/z: 266 [M+H]⁺.

Step 3: 2-(6-chloropyridin-3-yl)-8-(1-(2,2-difluoroethyl)-1H-pyrazolo[3,4-b]pyrazin-6-yl)-2,8-diazaspiro[4.5]decan-3-one: Followed the general procedure I using 2-(5-chloropyridin-2-yl)-2,8-diazaspiro[4.5]decan-3-one (110 mg, 0.414 mmol, 1.0 equiv.) and 6-chloro-1-(2,2-difluoroethyl)-1H-pyrazolo[3,4-b]pyrazine (108 mg, 0.497 mmol, 1.2 equiv.) as the starting materials to give 2-(6-chloropyridin-3-yl)-8-(1-(2,2-difluoroethyl)-1H-pyrazolo[3,4-b]pyrazin-6-yl)-2,8-diazaspiro[4.5]decan-3-one (63.6 mg, 34%) as a white solid. ¹H NMR (400 MHz, DMSO-d₆) δ 8.49 (s, 1H), 8.43 (d, J=2.6 Hz, 1H), 8.35 (d, J=9.1 Hz, 1H), 8.13 (s, 1H), 7.95 (dd, J=9.0, 2.7 Hz, 1H), 6.44 (tt, J=54.9, 3.8 Hz, 1H), 4.70 (td, J=15.0, 3.8 Hz, 2H), 3.97 (dt, J=14.0, 5.1 Hz, 2H), 3.89 (s, 2H), 3.69 (ddd, J=13.2, 7.2, 4.7 Hz, 2H), 2.67 (s, 2H), 1.82-1.67 (m, 4H). MS m/z: 448.2 [M+H]⁺.

### Example 893: 2-(5-chloropyridin-3-yl)-8-(1-(2,2-difluoroethyl)-1H-pyrazolo[3,4-b]pyrazin-6-yl)-2,8-diazaspiro[4.5]decan-3-one

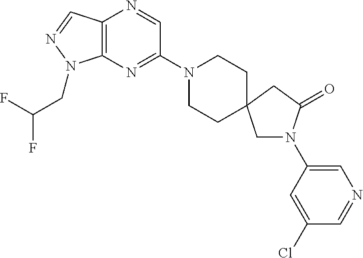 [SMILES: O=C1CC2(CCN(c3cnc4cnn(CC(F)F)c4n3)CC2)CN1c1cncc(Cl)c1]

Step 1: tert-butyl 2-(5-chloropyridin-3-yl)-3-oxo-2,8-diazaspiro[4.5]decane-8-carboxylate: Followed the general procedure Y using tert-butyl 3-oxo-2,8-diazaspiro[4.5]decane-8-carboxylate (100 mg, 339 μmol, 1.0 equiv.) and 3-bromo-5-chloropyridine (150 mg, 678 μmol, 2.0 equiv.) as the starting materials, Ephos Pd G4 (36 mg, 34 umol, 0.1 equiv.) as the catalyst to give tert-butyl 2-(5-chloropyridin-3-yl)-3-oxo-2,8-diazaspiro[4.5]decane-8-carboxylate (80 mg, 55%) as a colorless oil. MS m/z: 366 [M+H]⁺.

Step 2: 2-(5-chloropyridin-3-yl)-2,8-diazaspiro[4.5]decan-3-one hydrochloride: Followed the general procedure B using tert-butyl 2-(5-chloropyridin-3-yl)-3-oxo-2,8-diazaspiro[4.5]decane-8-carboxylate (80 mg) as the starting material to give the crude product 2-(5-chloropyridin-3-yl)-2,8-diazaspiro[4.5]decan-3-one hydrochloride (60 mg). MS m/z: 266 [M+H]⁺.

Step 3: 2-(5-chloropyridin-3-yl)-8-(1-(2,2-difluoroethyl)-1H-pyrazolo[3,4-b]pyrazin-6-yl)-2,8-diazaspiro[4.5]decan-3-one: Followed the general procedure I using 2-(5-chloropyridin-3-yl)-2,8-diazaspiro[4.5]decan-3-one hydrochloride (60 mg, 199 μmol, 1.0 equiv.) and 6-chloro-1-(2,2-difluoroethyl)-1H-pyrazolo[3,4-b]pyrazine (54 mg, 239 μmol, 1.2 equiv.) as the starting materials to give 2-(5-chloropyridin-3-yl)-8-(1-(2,2-difluoroethyl)-1H-pyrazolo[3,4-b]pyrazin-6-yl)-2,8-diazaspiro[4.5]decan-3-one (39 mg, 43%) as a white solid. ¹H NMR (400 MHz, DMSO-d₆) δ 8.86 (d, J=2.2 Hz, 1H), 8.51 (s, 1H), 8.41 (d, J=2.1 Hz, 1H), 8.31 (t, J=2.2 Hz, 1H), 8.14 (s, 1H), 6.44 (tt, J=54.9, 3.8 Hz, 1H), 4.70 (td, J=15.0, 3.8 Hz, 2H), 3.96-3.74 (m, 6H), 2.60 (s, 2H), 1.79-1.69 (m, 4H). MS m/z: 448.2 [M+H]⁺.

### Example 894: 8-(1-(2,2-difluoroethyl)-1H-pyrazolo[3,4-b]pyrazin-6-yl)-2-((2-(2,2,2-trifluoroethoxy)pyrimidin-5-yl)methyl)-2,8-diazaspiro[4.5]decan-3-one

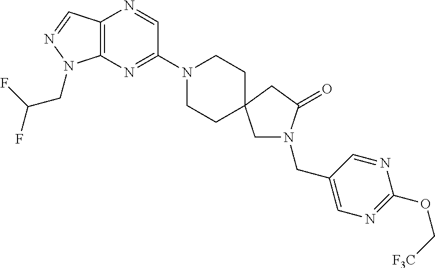 [SMILES: O=C1CC2(CCN(c3cnc4cnn(CC(F)F)c4n3)CC2)CN1Cc1cnc(OCC(F)(F)F)nc1]

Step 1: 5-methyl-2-(2,2,2-trifluoroethoxy)pyrimidine: Followed the general procedure E using 2-chloro-5-methylpyrimidine (1 g, 7.81 mmol, 1.0 equiv.) and CF₃CH₂OH (781 mg, 7.81 mmol, 1 equiv.) as the starting materials to give 5-methyl-2-(2,2,2-trifluoroethoxy)pyrimidine (650 mg, 43%) as a colorless oil. MS m/z: 193[M+H]⁺.

Step 2: 5-(chloromethyl)-2-(2,2,2-trifluoroethoxy)pyrimidine: Followed the general procedure W using 5-methyl-2-(2,2,2-trifluoroethoxy)pyrimidine (650 mg, 3.38 mmol, 1 equiv.) as the starting material, TCCA (787 mg, 3.38 mmol, 1.0 equiv.) as the reagent to give 5-(chloromethyl)-2-(2,2,2-trifluoroethoxy)pyrimidine (600 mg, 78%) as a colorless oil. MS m/z: 227[M+H]⁺.

Step 3: tert-butyl 3-oxo-2-((2-(2,2,2-trifluoroethoxy)pyrimidin-5-yl)methyl)-2,8-diazaspiro[4.5]decane-8-carboxylate: Followed the general procedure K using 5-(chloromethyl)-2-(2,2,2-trifluoroethoxy)pyrimidine (600 mg, 2.64 mmol, 1.0 equiv.) and tert-butyl 3-oxo-2,8-diazaspiro[4.5]decane-8-carboxylate (671 mg, 2.64 mmol, 1 equiv.) as the starting materials to give tert-butyl 3-oxo-2-((2-(2,2,2-trifluoroethoxy)pyrimidin-5-yl)methyl)-2,8-diazaspiro[4.5]decane-8-carboxylate (160 mg, 14%) as a colorless oil. MS m/z: 445[M+H]⁺.

Step 4: 2-((2-(2,2,2-trifluoroethoxy)pyrimidin-5-yl)methyl)-2,8-diazaspiro[4.5]decan-3-one hydrochloride: Followed the general procedure B using tert-butyl 3-oxo-2-((2-(2,2,2-trifluoroethoxy)pyrimidin-5-yl)methyl)-2,8-diazaspiro[4.5]decane-8-carboxylate (160 mg) as the starting material to give the crude product 2-((2-(2,2,2-trifluoroethoxy)pyrimidin-5-yl)methyl)-2,8-diazaspiro[4.5]decan-3-one hydrochloride (140 mg). MS m/z: 345 [M+H]⁺.

Step 5: 8-(1-(2,2-difluoroethyl)-1H-pyrazolo[3,4-b]pyrazin-6-yl)-2-((2-(2,2,2-trifluoroethoxy)pyrimidin-5-yl)methyl)-2,8-diazaspiro[4.5]decan-3-one: Followed the general procedure I using 2-((2-(2,2,2-trifluoroethoxy)pyrimidin-5-yl)methyl)-2,8-diazaspiro[4.5]decan-3-one hydrochloride (140 mg, 0.406 mmol, 1.0 equiv.) and 6-chloro-1-(2,2-difluoroethyl)-1H-pyrazolo[3,4-b]pyrazine (97.3 mg, 0.446 mmol, 1.1 equiv.) as the starting materials to give 8-(1-(2,2-difluoroethyl)-1H-pyrazolo[3,4-b]pyrazin-6-yl)-2-((2-(2,2,2-trifluoroethoxy)pyrimidin-5-yl)methyl)-2,8-diazaspiro[4.5]decan-3-one (50 mg, 23%) as a white solid. ¹H NMR (400 MHz, DMSO-d₆) δ 8.59 (s, 2H), 8.46 (s, 1H), 8.11 (s, 1H), 6.58-6.25 (m, 1H), 5.11-4.94 (m, 2H), 4.68 (td, J=15.0, 4.0 Hz, 2H), 4.40 (s, 2H), 3.91-3.79 (m, 2H), 3.68-3.60 (m, 2H), 3.29 (s, 1H), 3.19 (s, 1H), 2.35-2.33 (m, 2H), 1.67-1.57 (m, 4H). MS m/z: 527.15 [M+H]⁺.

### Example 895: 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decan-3-one

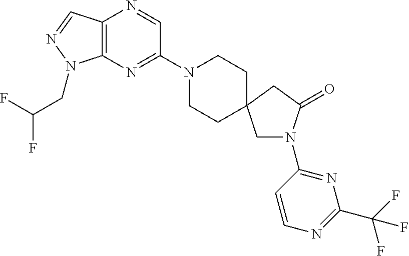 [SMILES: O=C1CC2(CCN(c3cnc4cnn(CC(F)F)c4n3)CC2)CN1c1ccnc(C(F)(F)F)n1]

Step 1: tert-butyl 3-oxo-2-(2-(trifluoromethyl)pyrimidin-4-yl)-2,8-diazaspiro[4.5]decane-8-carboxylate: Followed the general procedure I using tert-butyl 3-oxo-2,8-diazaspiro[4.5]decane-8-carboxylate (500 mg, 1.96 mmol, 1.0 equiv.) and 4-chloro-2-(trifluoromethyl)pyrimidine (359 mg, 1.96 mmol, 1.0 equiv.) as the starting materials to give tert-butyl 3-oxo-2-(2-(trifluoromethyl)pyrimidin-4-yl)-2,8-diazaspiro[4.5]decane-8-carboxylate (350 mg, 44%) as a white solid. MS m/z: 401 [M+H]⁺.

Step 2: 2-(2-(trifluoromethyl)pyrimidin-4-yl)-2,8-diazaspiro[4.5]decan-3-one: Followed the general procedure H using tert-butyl 3-oxo-2-(2-(trifluoromethyl)pyrimidin-4-yl)-2,8-diazaspiro[4.5]decane-8-carboxylate (350 mg) as the starting material to give the crude product 2-(2-(trifluoromethyl)pyrimidin-4-yl)-2,8-diazaspiro[4.5]decan-3-one (300 mg). MS m/z: 301 [M+H]⁺.

Step 3: 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decan-3-one: Followed the general procedure I using 2-(2-(trifluoromethyl)pyrimidin-4-yl)-2,8-diazaspiro[4.5]decan-3-one (80 mg, 266 μmol, 1.0 equiv.) and 6-chloro-1-(2,2-difluoroethyl)-1H-pyrazolo[3,4-b]pyrazine (65 mg, 293 μmol, 1.1 equiv.) as the starting materials to give 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decan-3-one (55 mg, 42%) as a white solid. ¹H NMR (400 MHz, DMSO-d₆) δ 8.89 (d, J=5.9 Hz, 1H), 8.55-8.48 (m, 2H), 8.14 (s, 1H), 6.44 (tt, J=54.9, 3.8 Hz, 1H), 4.70 (td, J=15.0, 3.8 Hz, 2H), 4.05 (dt, J=13.9, 4.8 Hz, 2H), 3.90 (s, 2H), 3.59 (ddd, J=13.2, 8.5, 3.9 Hz, 2H), 2.77 (s, 2H), 1.77 (dt, J=11.2, 5.1 Hz, 4H). MS m/z: 483.10 [M+H]⁺.

### Example 896: 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decan-1-one

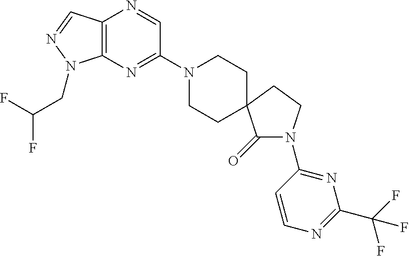 [SMILES: O=C1N(c2ccnc(C(F)(F)F)n2)CCC12CCN(c1cnc3cnn(CC(F)F)c3n1)CC2]

Step 1: tert-butyl 1-oxo-2-(2-(trifluoromethyl)pyrimidin-4-yl)-2,8-diazaspiro[4.5]decane-8-carboxylate: Followed the general procedure I using tert-butyl 1-oxo-2,8-diazaspiro[4.5]decane-8-carboxylate (500 mg, 1.96 mmol, 1.0 equiv.) and 4-chloro-2-(trifluoromethyl)pyrimidine (359 mg, 1.96 mmol, 1.0 equiv.) as the starting materials to give tert-butyl 1-oxo-2-(2-(trifluoromethyl)pyrimidin-4-yl)-2,8-diazaspiro[4.5]decane-8-carboxylate (350 mg, 44%) as a white solid. MS m/z: 401 [M+H]⁺.

Step 2: 2-(2-(trifluoromethyl)pyrimidin-4-yl)-2,8-diazaspiro[4.5]decan-1-one: Followed the general procedure H using tert-butyl 1-oxo-2-(2-(trifluoromethyl)pyrimidin-4-yl)-2,8-diazaspiro[4.5]decane-8-carboxylate (350 mg) as the starting material to give the crude product 2-(2-(trifluoromethyl)pyrimidin-4-yl)-2,8-diazaspiro[4.5]decan-1-one (300 mg). MS m/z: 301 [M+H]⁺.

Step 3: 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decan-1-one: Followed the general procedure I using 2-(2-(trifluoromethyl)pyrimidin-4-yl)-2,8-diazaspiro[4.5]decan-1-one (80 mg, 266 μmol, 1.0 equiv.) and 6-chloro-1-(2,2-difluoroethyl)-1H-pyrazolo[3,4-b]pyrazine (65 mg, 293 μmol, 1.1 equiv.) as the starting materials to give 8-(1-(2,2-difluoroethyl)-1H-pyrazolo[3,4-b]pyrazin-6-yl)-2-(2-(trifluoromethyl)pyrimidin-4-yl)-2,8-diazaspiro[4.5]decan-1-one (60 mg, 46%) as a white solid. ¹H NMR (400 MHz, DMSO-d₆) δ 8.89 (d, J=5.9 Hz, 1H), 8.53 (d, J=6.3 Hz, 2H), 8.16 (s, 1H), 6.45 (tt, J=54.9, 3.8 Hz, 1H), 4.71 (td, J=15.0, 3.8 Hz, 2H), 4.42 (dt, J=13.8, 4.2 Hz, 2H), 4.05 (t, J=7.0 Hz, 2H), 3.42-3.37 (m, 2H), 2.23 (t, J=7.1 Hz, 2H), 1.84 (ddd, J=14.9, 11.1, 4.1 Hz, 2H), 1.76 (dt, J=13.4, 3.7 Hz, 2H). MS m/z: 483.10 [M+H]⁺.

### Example 897: 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-1-one

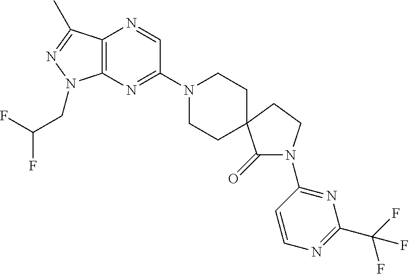 [SMILES: Cc1nn(CC(F)F)c2nc(N3CCC4(CC3)CCN(c3ccnc(C(F)(F)F)n3)C4=O)cnc12]

Step 1: 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-1-one: Followed the general procedure I using 2-(2-(trifluoromethyl)pyrimidin-4-yl)-2,8-diazaspiro[4.5]decan-1-one (80 mg, 265 μmol, 1.0 equiv.) and 6-chloro-1-(2,2-difluoroethyl)-3-methyl-1H-pyrazolo[3,4-b]pyrazine (69 mg, 292 μmol, 1.1 equiv.) as the starting materials to give 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-1-one (66.5 mg, 50%) as a white solid. ¹H NMR (400 MHz, DMSO-d₆) δ 8.89 (d, J=5.9 Hz, 1H), 8.53 (d, J=5.9 Hz, 1H), 8.44 (s, 1H), 6.42 (tt, J=55.0, 3.9 Hz, 1H), 4.62 (td, J=15.0, 3.9 Hz, 2H), 4.40 (d, J=13.8 Hz, 2H), 4.04 (t, J=7.1 Hz, 2H), 3.35 (s, 2H), 2.43 (s, 3H), 2.23 (t, J=7.1 Hz, 2H), 1.83 (td, J=12.3, 10.9, 4.0 Hz, 2H), 1.75 (d, J=13.5 Hz, 2H). MS m/z: 497.25 [M+H]⁺.

### Example 898: 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-3-one

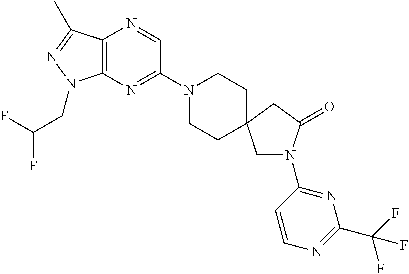 [SMILES: Cc1nn(CC(F)F)c2nc(N3CCC4(CC3)CC(=O)N(c3ccnc(C(F)(F)F)n3)C4)cnc12]

8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-3-one: Followed the general procedure I using 2-(2-(trifluoromethyl)pyrimidin-4-yl)-2,8-diazaspiro[4.5]decan-3-one hydrochloride (80 mg, 0.266 mmol, 1.0 equiv.) and 6-chloro-1-(2,2-difluoroethyl)-3-methyl-1H-pyrazolo[3,4-b]pyrazine (69 mg, 0.293 mmol, 1.1 equiv.) as the starting materials to give 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-3-one (77.2 mg, 58%) as a white solid. ¹H NMR (400 MHz, DMSO-d₆) δ 8.89 (d, J=6.0 Hz, 1H), 8.52 (d, J=5.9 Hz, 1H), 8.42 (s, 1H), 6.63-6.23 (m, 1H), 4.80-4.43 (m, 2H), 4.03 (dt, J=13.9, 4.9 Hz, 2H), 3.89 (s, 2H), 3.64-3.41 (m, 2H), 2.76 (s, 2H), 2.42 (s, 3H), 2.04-1.17 (m, 4H). MS m/z: 497.2 [M+H]⁺.

### Example 899: 7-methyl-2-(2-(2-methyl-6-(trifluoromethyl)pyrimidin-4-yl)-2,6-diazaspiro[3.4]octan-6-yl)-9-(oxetan-3-yl)-7,9-dihydro-8H-purin-8-one

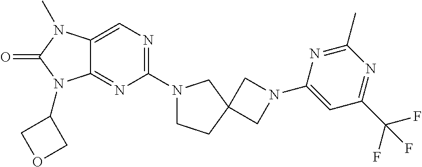 [SMILES: Cc1nc(N2CC3(CCN(c4ncc5c(n4)n(C4COC4)c(=O)n5C)C3)C2)cc(C(F)(F)F)n1]

Step 1: 7-methyl-2-(2-(2-methyl-6-(trifluoromethyl)pyrimidin-4-yl)-2,6-diazaspiro[3.4]octan-6-yl)-9-(oxetan-3-yl)-7,9-dihydro-8H-purin-8-one: Followed the general procedure I using 2-chloro-7-methyl-9-(oxetan-3-yl)-7,9-dihydro-8H-purin-8-one (100 mg, 0.416 mmol, 1.0 equiv.) and 2-(2-methyl-6-(trifluoromethyl)pyrimidin-4-yl)-2,6-diazaspiro[3.4]octane (124 mg, 0.458 mmol, 1.1 equiv.) as the starting materials to give 7-methyl-2-(2-(2-methyl-6-(trifluoromethyl)pyrimidin-4-yl)-2,6-diazaspiro[3.4]octan-6-yl)-9-(oxetan-3-yl)-7,9-dihydro-8H-purin-8-one (24.6 mg, 12%) as a white solid. ¹H NMR (400 MHz, DMSO-d₆) δ 8.06 (s, 1H), 6.64 (s, 1H), 5.52-5.37 (m, 1H), 5.38-5.25 (m, 2H), 4.84-4.68 (m, 2H), 4.11 (q, J=9.3 Hz, 4H), 3.78-3.68 (m, 2H), 3.57 (t, J=7.0 Hz, 2H), 3.27 (s, 3H), 2.42 (s, 3H), 2.23 (t, J=6.9 Hz, 2H). MS m/z: 477.35 [M+H]⁺.

### Example 900: 8-(1-(2,2-difluoroethyl)-1H-pyrazolo[3,4-d]pyrimidin-6-yl)-2-(2-(trifluoromethyl)pyrimidin-4-yl)-2,8-diazaspiro[4.5]decan-1-one

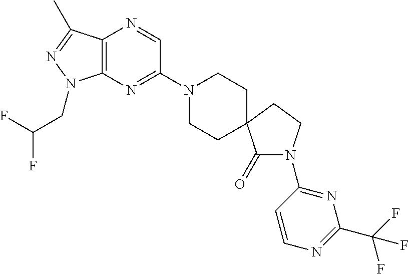 [SMILES: Cc1nn(CC(F)F)c2nc(N3CCC4(CC3)CCN(c3ccnc(C(F)(F)F)n3)C4=O)cnc12]

Step 1: 8-(1-(2,2-difluoroethyl)-1H-pyrazolo[3,4-d]pyrimidin-6-yl)-2-(2-(trifluoromethyl)pyrimidin-4-yl)-2,8-diazaspiro[4.5]decan-1-one: Followed the general procedure I using 2-(2-(trifluoromethyl)pyrimidin-4-yl)-2,8-diazaspiro[4.5]decan-1-one (80 mg, 0.20 mmol, 1.0 equiv.) and 6-chloro-1-(2,2-difluoroethyl)-1H-pyrazolo[3,4-d]pyrimidine (48 mg, 0.22 mmol, 1.1 equiv.) as the starting materials to give 8-(1-(2,2-difluoroethyl)-1H-pyrazolo[3,4-d]pyrimidin-6-yl)-2-(2-(trifluoromethyl)pyrimidin-4-yl)-2,8-diazaspiro[4.5]decan-1-one (19 mg, 15%) as a white solid. ¹H NMR (400 MHz, DMSO-d₆) δ 8.96-8.86 (m, 2H), 8.53 (d, J=5.9 Hz, 1H), 8.09 (s, 1H), 6.63-6.30 (m, 1H), 4.74-4.60 (m, 4H), 4.05 (t, J=7.1 Hz, 2H), 3.38 (s, 2H), 2.24 (t, J=7.1 Hz, 2H), 1.80-1.70 (m, 4H). MS m/z: 483.25 [M+H]⁺.

### Example 901: 8-(1-(2,2-difluoroethyl)-1H-pyrazolo[3,4-d]pyrimidin-6-yl)-2-(2-(trifluoromethyl)pyrimidin-4-yl)-2,8-diazaspiro[4.5]decan-3-one

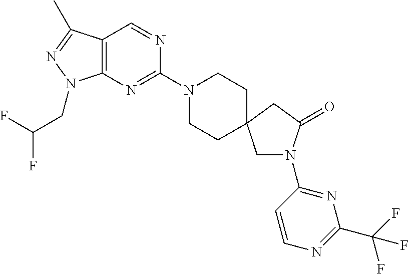 [SMILES: Cc1nn(CC(F)F)c2nc(N3CCC4(CC3)CC(=O)N(c3ccnc(C(F)(F)F)n3)C4)ncc12]

Step 1: 8-(1-(2,2-difluoroethyl)-1H-pyrazolo[3,4-d]pyrimidin-6-yl)-2-(2-(trifluoromethyl)pyrimidin-4-yl)-2,8-diazaspiro[4.5]decan-3-one: Followed the general procedure I using 2-(2-(trifluoromethyl)pyrimidin-4-yl)-2,8-diazaspiro[4.5]decan-3-one hydrochloride (80 mg, 0.266 mmol, 1.0 equiv.) and 6-chloro-1-(2,2-difluoroethyl)-1H-pyrazolo[3,4-d]pyrimidine (63.9 mg, 0.293 mmol, 1.1 equiv.) as the starting materials to give 8-(1-(2,2-difluoroethyl)-1H-pyrazolo[3,4-d]pyrimidin-6-yl)-2-(2-(trifluoromethyl)pyrimidin-4-yl)-2,8-diazaspiro[4.5]decan-3-one (52.3 mg, 41%) as a white solid. ¹H NMR (400 MHz, DMSO-d₆) δ 9.01-8.84 (m, 2H), 8.52 (d, J=6.0 Hz, 1H), 8.08 (s, 1H), 6.44 (tt, J=54.8, 3.6 Hz, 1H), 4.67 (td, J=15.0, 3.6 Hz, 2H), 4.26-4.06 (m, 2H), 3.89 (s, 2H), 3.70-3.56 (m, 2H), 2.76 (s, 2H), 1.83-1.62 (m, 4H). MS m/z: 483.1 [M+H]⁺.

### Example 902: 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-1-one

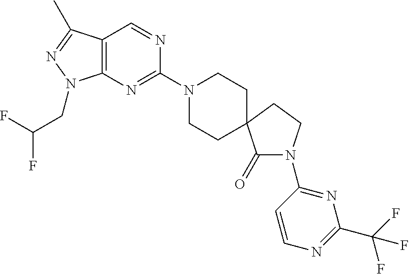 [SMILES: Cc1nn(CC(F)F)c2nc(N3CCC4(CC3)CCN(c3ccnc(C(F)(F)F)n3)C4=O)ncc12]

Step 1: 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-1-one: Followed the general procedure I using 2-(2-(trifluoromethyl)pyrimidin-4-yl)-2,8-diazaspiro[4.5]decan-3-one hydrochloride (120 mg, 0.40 mmol, 1.0 equiv.) and 6-chloro-1-(2,2-difluoroethyl)-3-methyl-1H-pyrazolo[3,4-b]pyrazine (103 mg, 0.44 mmol, 1.1 equiv.) as the starting materials to give 8-(1-(2,2-difluoroethyl)-3-methyl-1H-pyrazolo[3,4-b]pyrazin-6-yl)-2-(2-(trifluoromethyl)pyrimidin-4-yl)-2,8-diazaspiro[4.5]decan-1-one (72.3 mg, 35%) as a white solid. ¹H NMR (400 MHz, DMSO-d₆) δ 8.83 (s, 1H), 8.78 (d, J=6.0 Hz, 1H), 8.45 (d, J=5.9 Hz, 1H), 6.53-6.10 (m, 1H), 5.47-4.28 (m, 4H), 3.99 (t, J=7.1 Hz, 2H), 3.48-3.01 (m, 2H), 2.39 (s, 3H), 2.21 (t, J=7.1 Hz, 2H), 2.04-1.02 (m, 4H). MS m/z: 497.15 [M+H]⁺.

### Example 903: 8-(1-(2,2-difluoroethyl)-3-methyl-1H-pyrazolo[3,4-d]pyrimidin-6-yl)-2-(2-(trifluoromethyl)pyrimidin-4-yl)-2,8-diazaspiro[4.5]decan-3-one

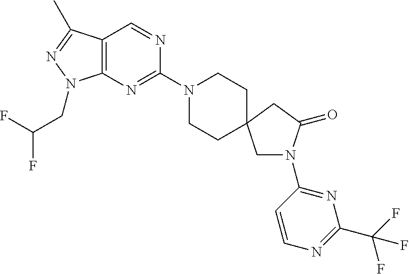 [SMILES: Cc1nn(CC(F)F)c2nc(N3CCC4(CC3)CC(=O)N(c3ccnc(C(F)(F)F)n3)C4)ncc12]

Step 1: 8-(1-(2,2-difluoroethyl)-3-methyl-1H-pyrazolo[3,4-d]pyrazin-6-yl)-2-(2-(trifluoromethyl)pyrimidin-4-yl)-2,8-diazaspiro[4.5]decan-1-one: Followed the general procedure I using 2-(2-(trifluoromethyl)pyrimidin-4-yl)-2,8-diazaspiro[4.5]decan-3-one hydrochloride (120 mg, 0.40 mmol, 1.0 equiv.) and 6-chloro-1-(2,2-difluoroethyl)-3-methyl-1H-pyrazolo[3,4-d]pyrimidine (103 mg, 0.44 mmol, 1.1 equiv.) as the starting materials to give 8-(1-(2,2-difluoroethyl)-3-methyl-1H-pyrazolo[3,4-d]pyrimidin-6-yl)-2-(2-(trifluoromethyl)pyrimidin-4-yl)-2,8-diazaspiro[4.5]decan-3-one (68.6 mg, 34%) as a white solid. ¹H NMR (400 MHz, DMSO-d₆) δ 8.89 (d, J=6.0 Hz, 2H), 8.51 (d, J=5.9 Hz, 1H), 6.70-6.19 (m, 1H), 4.57 (td, J=14.9, 3.8 Hz, 2H), 4.20 (d, J=13.8 Hz, 2H), 3.88 (s, 2H), 3.69-3.33 (m, 2H), 2.75 (s, 2H), 2.42 (s, 3H), 2.31-0.68 (m, 4H). MS m/z: 497.1 [M+H]⁺.

### Example 904: 8-(1-(2,2-difluoroethyl)-1H-pyrazolo[3,4-b]pyrazin-6-yl)-1-ethyl-2-(2-(trifluoromethyl)pyrimidin-4-yl)-2,8-diazaspiro[4.5]decan-3-one

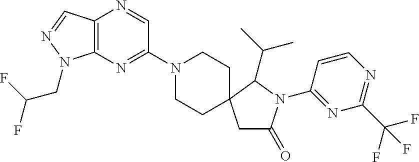 [SMILES: CC(C)C1N(c2ccnc(C(F)(F)F)n2)C(=O)CC12CCN(c1cnc3cnn(CC(F)F)c3n1)CC2]

Step 1: 8-(1-(2,2-difluoroethyl)-1H-pyrazolo[3,4-b]pyrazin-6-yl)-1-ethyl-2-(2-(trifluoromethyl)pyrimidin-4-yl)-2,8-diazaspiro[4.5]decan-3-one: Followed the general procedure I using 1-ethyl-2-(2-(trifluoromethyl)pyrimidin-4-yl)-2,8-diazaspiro[4.5]decan-3-one (75 mg, 0.228 mmol, 1 equiv) and 6-chloro-1-(2,2-difluoroethyl)-1H-pyrazolo[3,4-b]pyrazine (59.9 mg, 0.274 mmol, 1.2 equiv) as the starting materials to give 8-(1-(2,2-difluoroethyl)-1H-pyrazolo[3,4-b]pyrazin-6-yl)-1-ethyl-2-(2-(trifluoromethyl)pyrimidin-4-yl)-2,8-diazaspiro[4.5]decan-3-one (40 mg, 78%) as a white solid. ¹H NMR (400 MHz, DMSO-d₆) δ 8.90 (d, J=5.9 Hz, 1H), 8.54 (d, J=5.9 Hz, 1H), 8.48 (s, 1H), 8.13 (s, 1H), 6.44 (tt, J=54.9, 3.9 Hz, 1H), 4.69 (td, J=15.0, 3.9 Hz, 2H), 4.45 (t, J=5.5 Hz, 1H), 4.25 (dd, J=40.5, 13.7 Hz, 2H), 3.53-3.41 (m, 1H), 3.29-3.25 (m, 1H), 2.93-2.75 (m, 2H), 2.05-1.91 (m, 1H), 1.88-1.77 (m, 2H), 1.72-1.61 (m, 3H), 0.82 (t, J=7.5 Hz, 3H). MS m/z: 511.1 [M+H]⁺.

### Example 905: 8-(1-(2,2-difluoroethyl)-1H-pyrazolo[3,4-b]pyrazin-6-yl)-1-ethyl-2-(2-(trifluoromethyl)pyrimidin-5-yl)-2,8-diazaspiro[4.5]decan-3-one

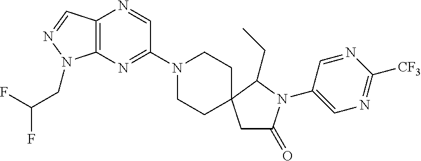 [SMILES: CCC1N(c2cnc(C(F)(F)F)nc2)C(=O)CC12CCN(c1cnc3cnn(CC(F)F)c3n1)CC2]

Step 1: tert-butyl 1-ethyl-3-oxo-2-(2-(trifluoromethyl)pyrimidin-5-yl)-2,8-diazaspiro[4.5]decane-8-carboxylate: Followed the general procedure Y using tert-butyl 1-ethyl-3-oxo-2,8-diazaspiro[4.5]decane-8-carboxylate (120 mg, 0.425 mmol, 1 equiv.) and 5-bromo-2-(trifluoromethyl)pyrimidine (193 mg, 0.85 mmol, 2 equiv.) as the starting materials to give tert-butyl 1-ethyl-3-oxo-2-[2-(trifluoromethyl)pyrimidin-5-yl]-2,8-diazaspiro[4.5]decane-8-carboxylate (104 mg, 57%) as a yellow oil. MS m/z: 428 [M+H]⁺.

Step 2: 1-ethyl-2-(2-(trifluoromethyl)pyrimidin-5-yl)-2,8-diazaspiro[4.5]decan-3-one hydrochloride: Followed the general procedure B using tert-butyl 1-ethyl-3-oxo-2-[2-(trifluoromethyl)pyrimidin-5-yl]-2,8-diazaspiro[4.5]decane-8-carboxylate (104 mg) as the starting material to give the crude product 1-ethyl-2-(2-(trifluoromethyl)pyrimidin-5-yl)-2,8-diazaspiro[4.5]decan-3-one hydrochloride (80 mg). MS m/z: 328 [M+H]⁺.

Step 3: 8-(1-(2,2-difluoroethyl)-1H-pyrazolo[3,4-b]pyrazin-6-yl)-1-ethyl-2-(2-(trifluoromethyl)pyrimidin-5-yl)-2,8-diazaspiro[4.5]decan-3-one: Followed the general procedure I using 1-ethyl-2-[2-(trifluoromethyl)pyrimidin-5-yl]-2,8-diazaspiro[4.5]decan-3-one hydrochloride (80 mg, 0.24 mmol, 1 equiv.) and 6-chloro-1-(2,2-difluoroethyl)pyrazolo[3,4-b]pyrazine (63.9 mg, 0.29 mmol, 1.2 equiv.) as the starting materials to give 8-[1-(2,2-difluoroethyl)-1H-pyrazolo[3,4-b]pyrazin-6-yl]-1-ethyl-2-[2-(trifluoromethyl)pyrimidin-5-yl]-2,8-diazaspiro[4.5]decan-3-one (26.4 mg, 21%) as a white solid. ¹H NMR (400 MHz, DMSO-d₆) δ 9.40 (s, 2H), 8.51 (s, 1H), 8.15 (s, 1H), 6.68-6.21 (m, 1H), 4.84-4.62 (m, 2H), 4.43 (t, J=5.5 Hz, 1H), 4.30-4.09 (m, 2H), 3.65-3.52 (m, 1H), 3.50-3.40 (m, 1H), 2.80 (d, J=17.3 Hz, 1H), 2.63 (d, J=17.3 Hz, 1H), 2.04-1.57 (m, 6H), 0.79 (t, J=7.4 Hz, 3H). MS m/z: 511.2 [M+H]⁺.

### Example 906: 8-(1-(2,2-difluoroethyl)-1H-pyrazolo[3,4-b]pyrazin-6-yl)-1-ethyl-3-(2-(trifluoromethyl)pyrimidin-4-yl)-1,3,8-triazaspiro[4.5]decane-2,4-dione

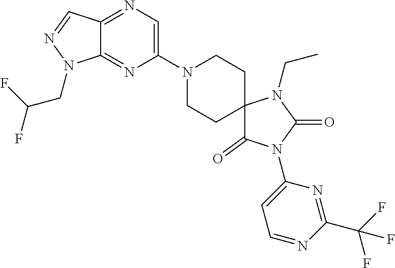 [SMILES: CCN1C(=O)N(c2ccnc(C(F)(F)F)n2)C(=O)C12CCN(c1cnc3cnn(CC(F)F)c3n1)CC2]

Step 1: tert-butyl 2,4-dioxo-3-(2-(trifluoromethyl)pyrimidin-4-yl)-1,3,8-triazaspiro[4.5]decane-8-carboxylate: Followed the general procedure I using 4-chloro-2-(trifluoromethyl)pyrimidine (170 mg, 0.931 mmol, 1 equiv) and tert-butyl 2,4-dioxo-1,3,8-triazaspiro[4.5]decane-8-carboxylate (300 mg, 1.117 mmol, 1.2 equiv) as the starting materials to give tert-butyl 2,4-dioxo-3-[2-(trifluoromethyl)pyrimidin-4-yl]-1,3,8-triazaspiro[4.5]decane-8-carboxylate (150 mg, 38%) as a white solid. MS m/z: 416 [M+H]⁺.

Step 2: tert-butyl 1-ethyl-2,4-dioxo-3-(2-(trifluoromethyl)pyrimidin-4-yl)-1,3,8-triazaspiro[4.5]decane-8-carboxylate: Followed the general procedure K using tert-butyl 2,4-dioxo-3-[2-(trifluoromethyl)pyrimidin-4-yl]-1,3,8-triazaspiro[4.5]decane-8-carboxylate (100 mg, 0.241 mmol, 1 equiv) and ethyl iodide (45 mg, 0.29 mmol, 1.2 equiv) as the starting materials to give tert-butyl 1-ethyl-2,4-dioxo-3-[2-(trifluoromethyl)pyrimidin-4-yl]-1,3,8-triazaspiro[4.5]decane-8-carboxylate (90 mg, 84%) as a white solid. MS m/z: 444 [M+H]⁺.

Step 3: 1-ethyl-3-(2-(trifluoromethyl)pyrimidin-4-yl)-1,3,8-triazaspiro[4.5]decane-2,4-dione hydrochloride: Followed the general procedure B using tert-butyl 1-ethyl-2,4-dioxo-3-[2-(trifluoromethyl)pyrimidin-4-yl]-1,3,8-triazaspiro[4.5]decane-8-carboxylate (95 mg, 0.214 mmol, 1 equiv) as the starting material to give the crude product 1-ethyl-3-(2-(trifluoromethyl)pyrimidin-4-yl)-1,3,8-triazaspiro[4.5]decane-2,4-dione hydrochloride (70 mg). MS m/z: 344 [M+H]⁺.

Step 4: 8-(1-(2,2-difluoroethyl)-1H-pyrazolo[3,4-b]pyrazin-6-yl)-1-ethyl-3-(2-(trifluoromethyl)pyrimidin-4-yl)-1,3,8-triazaspiro[4.5]decane-2,4-dione: Followed the general procedure I using 1-ethyl-3-[2-(trifluoromethyl)pyrimidin-4-yl]-1,3,8-triazaspiro[4.5]decane-2,4-dione hydrochloride (70 mg, 0.204 mmol, 1 equiv) and 6-chloro-1-(2,2-difluoroethyl)-1H-pyrazolo[3,4-b]pyrazine (53.48 mg, 0.245 mmol, 1.2 equiv) as the starting materials to give 8-(1-(2,2-difluoroethyl)-1H-pyrazolo[3,4-b]pyrazin-6-yl)-1-ethyl-3-(2-(trifluoromethyl)pyrimidin-4-yl)-1,3,8-triazaspiro[4.5]decane-2,4-dione (40 mg, 36%) as a white solid. ¹H NMR (400 MHz, DMSO-d₆) δ 9.24 (d, J=5.5 Hz, 1H), 8.53 (s, 1H), 8.15 (s, 1H), 8.02 (d, J=5.5 Hz, 1H), 6.45 (tt, J=54.8, 3.8 Hz, 1H), 4.81-4.58 (m, 4H), 3.70-3.52 (m, 2H), 3.39-3.32 (m, 2H), 2.21-2.04 (m, 4H), 1.16 (t, 3H). MS m/z: 526.2 [M+H]⁺.

### Example 907: 8-(1-(oxetan-3-yl)-1H-pyrazolo[3,4-b]pyrazin-6-yl)-2-(2-(trifluoromethyl)pyrimidin-4-yl)-2,8-diazaspiro[4.5]decan-3-one

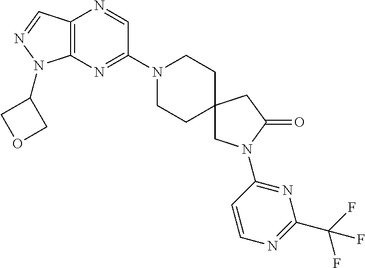 [SMILES: O=C1CC2(CCN(c3cnc4cnn(C5COC5)c4n3)CC2)CN1c1ccnc(C(F)(F)F)n1]

8-(1-(oxetan-3-yl)-1H-pyrazolo[3,4-b]pyrazin-6-yl)-2-(2-(trifluoromethyl)pyrimidin-4-yl)-2,8-diazaspiro[4.5]decan-3-one: Followed the general procedure I using 2-(2-(trifluoromethyl)pyrimidin-4-yl)-2,8-diazaspiro[4.5]decan-3-one hydrochloride (80 mg, 0.266 mmol, 1.0 equiv.) and 6-chloro-1-(oxetan-3-yl)-1H-pyrazolo[3,4-b]pyrimidine (62 mg, 0.293 mmol, 1.1 equiv.) as the starting materials to give 8-(1-(oxetan-3-yl)-1H-pyrazolo[3,4-b]pyrazin-6-yl)-2-(2-(trifluoromethyl)pyrimidin-4-yl)-2,8-diazaspiro[4.5]decan-3-one (63.6 mg, 49%) as a white solid. ¹H NMR (400 MHz, DMSO-d₆) δ 8.89 (s, 1H), 8.88 (s, 1H), 8.52 (s, 1H), 8.51 (s, 1H), 6.40 (t, J=3.8 Hz, 1H), 4.57 (td, J=14.9, 3.8 Hz, 4H), 4.20 (d, J=13.8 Hz, 2H), 3.88 (s, 2H), 3.64 (dd, J=13.8, 6.2 Hz, 2H), 2.75 (s, 2H), 1.70 (d, J=4.8 Hz, 4H). MS m/z: 475.2 [M+H]⁺.

### Example 908: 8-(1-(oxetan-3-yl)-1H-pyrazolo[3,4-d]pyrimidin-6-yl)-2-(2-(trifluoromethyl)pyrimidin-4-yl)-2,8-diazaspiro[4.5]decan-1-one

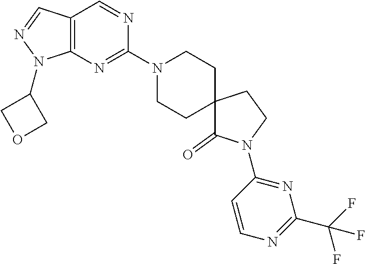 [SMILES: O=C1N(c2ccnc(C(F)(F)F)n2)CCC12CCN(c1ncc3cnn(C4COC4)c3n1)CC2]

8-(1-(oxetan-3-yl)-1H-pyrazolo[3,4-d]pyrimidin-6-yl)-2-(2-(trifluoromethyl)pyrimidin-4-yl)-2,8-diazaspiro[4.5]decan-1-one: Followed the general procedure I using 2-(2-(trifluoromethyl)pyrimidin-4-yl)-2,8-diazaspiro[4.5]decan-1-one hydrochloride (80 mg, 0.266 mmol, 1.0 equiv.) and 6-chloro-1-(oxetan-3-yl)-1H-pyrazolo[3,4-d]pyrimidine (62 mg, 0.293 mmol, 1.1 equiv.) as the starting materials to give 8-(1-(oxetan-3-yl)-1H-pyrazolo[3,4-d]pyrimidin-6-yl)-2-(2-(trifluoromethyl)pyrimidin-4-yl)-2,8-diazaspiro[4.5]decan-1-one (44.9 mg, 35%) as a white solid. ¹H NMR (400 MHz, DMSO-d₆) δ 8.89 (d, J=6.0 Hz, 1H), 8.62-8.36 (m, 2H), 8.21 (s, 1H), 5.99-5.80 (m, 1H), 5.06 (t, J=6.4 Hz, 2H), 5.00-4.86 (m, 2H), 4.54-4.30 (m, 2H), 4.04 (t, J=7.1 Hz, 2H), 3.41-3.35 (m, 2H), 2.23 (t, J=7.1 Hz, 2H), 1.87-1.79 (m, 2H), 1.79-1.70 (m, 2H). MS m/z: 475.25 [M+H]⁺.

### Example 909: 1-ethyl-8-(1-(oxetan-3-yl)-1H-pyrazolo[3,4-b]pyrazin-6-yl)-3-(2-(trifluoromethyl)pyrimidin-4-yl)-1,3,8-triazaspiro[4.5]decane-2,4-dione

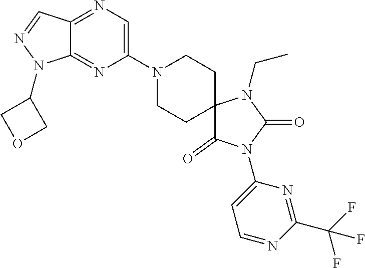 [SMILES: CCN1C(=O)N(c2ccnc(C(F)(F)F)n2)C(=O)C12CCN(c1cnc3cnn(C4COC4)c3n1)CC2]

1-ethyl-8-(1-(oxetan-3-yl)-1H-pyrazolo[3,4-b]pyrazin-6-yl)-3-(2-(trifluoromethyl)pyrimidin-4-yl)-1,3,8-triazaspiro[4.5]decane-2,4-dione: Followed the general procedure I using 1-ethyl-3-[2-(trifluoromethyl)pyrimidin-4-yl]-1,3,8-triazaspiro[4.5]decane-2,4-dione (70 mg, 0.204 mmol, 1 equiv) and 6-chloro-1-(oxetan-3-yl)-1H-pyrazolo[3,4-b]pyrazine (51.7 mg, 0.245 mmol, 1.2 equiv) as the starting materials to give 1-ethyl-8-(1-(oxetan-3-yl)-1H-pyrazolo[3,4-b]pyrazin-6-yl)-3-(2-(trifluoromethyl)pyrimidin-4-yl)-1,3,8-triazaspiro[4.5]decane-2,4-dione (40 mg, 36%) as a white solid. ¹H NMR (400 MHz, DMSO-d₆) δ 9.24 (d, J=5.4 Hz, 1H), 8.51 (s, 1H), 8.21 (s, 1H), 8.02 (d, J=5.5 Hz, 1H), 5.99-5.88 (m, 1H), 5.10-5.02 (m, 2H), 5.02-4.94 (m, 2H), 4.64 (d, J=13.6 Hz, 2H), 3.67-3.55 (m, 2H), 3.37-3.30 (m, 2H), 2.19-2.04 (m, 4H), 1.15 (t, J=7.0 Hz, 3H). MS m/z: 518.1 [M+H]⁺.

### Example 910: 2-(1-(2,2-difluoroethyl)-1H-pyrazolo[3,4-b]pyrazin-6-yl)-7-(6-(trifluoromethyl)pyridin-3-yl)-2,7-diazaspiro[3.5]nonan-6-one

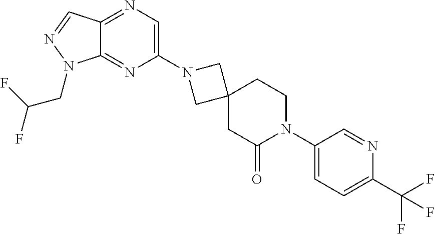 [SMILES: O=C1CC2(CCN1c1ccc(C(F)(F)F)nc1)CN(c1cnc3cnn(CC(F)F)c3n1)C2]

Step 1: 2-benzhydryl-7-(6-(trifluoromethyl)pyridin-3-yl)-2,7-diazaspiro[3.5]nonan-6-one: Followed the general procedure K using 2-benzhydryl-2,7-diazaspiro[3.5]nonan-6-one (210 mg, 0.686 mmol, 1.0 equiv.) and 5-fluoro-2-(trifluoromethyl)pyridine (125 mg, 0.755 mmol, 1.1 equiv.) as the starting materials to give 2-benzhydryl-7-(6-(trifluoromethyl)pyridin-3-yl)-2,7-diazaspiro[3.5]nonan-6-one (110 mg, 35%) as a colorless oil. MS m/z: 452[M+H]⁺.

Step 2: 7-(6-(trifluoromethyl)pyridin-3-yl)-2,7-diazaspiro[3.5]nonan-6-one: Followed the general procedure U using 2-benzhydryl-7-(6-(trifluoromethyl)pyridin-3-yl)-2,7-diazaspiro[3.5]nonan-6-one (110 mg, 0.244 mmol, 1.0 equiv.) as the starting material to give the product 7-(6-(trifluoromethyl)pyridin-3-yl)-2,7-diazaspiro[3.5]nonan-6-one (60 mg, 86%). MS m/z: 286 [M+H]⁺.

Step 3: 2-(1-(2,2-difluoroethyl)-1H-pyrazolo[3,4-b]pyrazin-6-yl)-7-(6-(trifluoromethyl)pyridin-3-yl)-2,7-diazaspiro[3.5]nonan-6-one: Followed the general procedure I using 7-(6-(trifluoromethyl)pyridin-3-yl)-2,7-diazaspiro[3.5]nonan-6-one (60 mg, 0.211 mmol, 1.0 equiv.) and 6-chloro-1-(2,2-difluoroethyl)-1H-pyrazolo[3,4-b]pyrazine (50.5 mg, 0.232 mmol, 1.1 equiv.) as the starting materials to give 2-(1-(2,2-difluoroethyl)-1H-pyrazolo[3,4-b]pyrazin-6-yl)-7-(6-(trifluoromethyl)pyridin-3-yl)-2,7-diazaspiro[3.5]nonan-6-one (37.9 mg, 39%) as a white solid. ¹H NMR (400 MHz, DMSO-d₆) δ 8.80 (d, J=2.4 Hz, 1H), 8.16 (s, 1H), 8.09 (dd, J=8.4, 2.4 Hz, 1H), 8.01-7.93 (m, 2H), 6.45 (tt, J=54.8, 3.6 Hz, 1H), 4.68 (td, J=15.0, 3.6 Hz, 2H), 4.15 (d, J=8.8 Hz, 2H), 4.06 (d, J=8.8 Hz, 2H), 3.85 (t, J=6.0 Hz, 2H), 2.88 (s, 2H), 2.26 (t, J=6.0 Hz, 2H). MS m/z: 468.15 [M+H]⁺.

### Example 911: 7-(1-(2,2-difluoroethyl)-1H-pyrazolo[3,4-b]pyrazin-6-yl)-2-(5-(trifluoromethyl)pyridin-2-yl)-2,7-diazaspiro[3.5]nonan-6-one

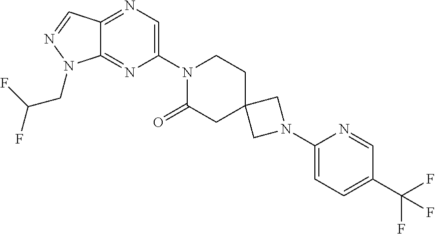 [SMILES: O=C1CC2(CCN1c1cnc3cnn(CC(F)F)c3n1)CN(c1ccc(C(F)(F)F)cn1)C2]

Step 1: 7-(1-(2,2-difluoroethyl)-1H-pyrazolo[3,4-b]pyrazin-6-yl)-2-(5-(trifluoromethyl)pyridin-2-yl)-2,7-diazaspiro[3.5]nonan-6-one: Followed the general procedure I using 2-fluoro-5-(trifluoromethyl)pyridine (50 mg, 0.229 mmol, 1.0 equiv.) and 7-(1-(2,2-difluoroethyl)-1H-pyrazolo[3,4-b]pyrazin-6-yl)-2,7-diazaspiro[3.5]nonan-6-one (78 mg, 0.275 mmol, 1.2 equiv.) as the starting materials to give 7-(1-(2,2-difluoroethyl)-1H-pyrazolo[3,4-b]pyrazin-6-yl)-2-(5-(trifluoromethyl)pyridin-2-yl)-2,7-diazaspiro[3.5]nonan-6-one (19 mg, 13%) as a white solid. ¹H NMR (400 MHz, DMSO-d₆) δ 9.03 (d, J=1.9 Hz, 1H), 8.50 (d, J=1.9 Hz, 1H), 8.40 (s, 1H), 7.80 (dt, J=8.9, 2.2 Hz, 1H), 6.69-6.36 (m, 2H), 4.98-4.84 (m, 2H), 4.03 (dd, J=13.8, 7.5 Hz, 4H), 3.94 (d, J=8.5 Hz, 2H), 3.00-2.95 (m, 2H), 2.28 (t, J=6.1 Hz, 2H). MS m/z: 468.05 [M+H]⁺.

### Example 912: 2-(1-(2,2-difluoroethyl)-1H-pyrazolo[3,4-b]pyrazin-6-yl)-7-(5-(trifluoromethyl)pyridin-2-yl)-2,7-diazaspiro[3.5]nonan-6-one

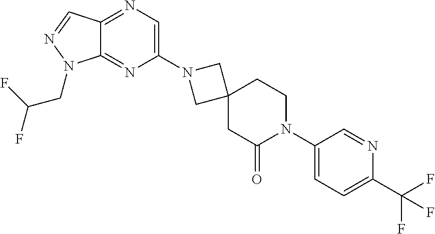 [SMILES: O=C1CC2(CCN1c1ccc(C(F)(F)F)nc1)CN(c1cnc3cnn(CC(F)F)c3n1)C2]

Step 1: 2-benzhydryl-7-(5-(trifluoromethyl)pyridin-2-yl)-2,7-diazaspiro[3.5]nonan-6-one: Followed the general procedure K using 2-benzhydryl-2,7-diazaspiro[3.5]nonan-6-one (210 mg, 0.686 mmol, 1.0 equiv.) and 2-fluoro-5-(trifluoromethyl)pyridine (125 mg, 0.755 mmol, 1.1 equiv.) as the starting materials to give 2-benzhydryl-7-(5-(trifluoromethyl)pyridin-2-yl)-2,7-diazaspiro[3.5]nonan-6-one (110 mg, 35%) as a colorless oil. MS m/z: 452[M+H]⁺.

Step 2: 7-(5-(trifluoromethyl)pyridin-2-yl)-2,7-diazaspiro[3.5]nonan-6-one: Followed the general procedure U using 2-benzhydryl-7-(5-(trifluoromethyl)pyridin-2-yl)-2,7-diazaspiro[3.5]nonan-6-one (110 mg, 0.244 mmol, 1.0 equiv.) as the starting material to give the product 7-(5-(trifluoromethyl)pyridin-2-yl)-2,7-diazaspiro[3.5]nonan-6-one (60 mg, 86%). MS m/z: 286 [M+H]⁺

Step 3: 2-(1-(2,2-difluoroethyl)-1H-pyrazolo[3,4-b]pyrazin-6-yl)-7-(5-(trifluoromethyl)pyridin-2-yl)-2,7-diazaspiro[3.5]nonan-6-one: Followed the general procedure I using 7-(5-(trifluoromethyl)pyridin-2-yl)-2,7-diazaspiro[3.5]nonan-6-one (60 mg, 0.211 mmol, 1.0 equiv.) and 6-chloro-1-(2,2-difluoroethyl)-1H-pyrazolo[3,4-b]pyrazine (50.5 mg, 0.232 mmol, 1.1 equiv.) as the starting materials to give 2-(1-(2,2-difluoroethyl)-1H-pyrazolo[3,4-b]pyrazin-6-yl)-7-(5-(trifluoromethyl)pyridin-2-yl)-2,7-diazaspiro[3.5]nonan-6-one (23.6 mg, 24%) as a white solid. ¹H NMR (400 MHz, DMSO-d₆) δ 8.86 (d, J=2.4 Hz, 1H), 8.24-8.18 (m, 1H), 8.16 (s, 1H), 8.07 (d, J=8.8 Hz, 1H), 7.95 (s, 1H), 6.45 (tt, J=54.8, 3.6 Hz, 1H), 4.68 (td, J=15.0, 3.6 Hz, 2H), 4.18-4.10 (m, 2H), 4.09-3.96 (m, 4H), 2.97 (s, 2H), 2.26 (t, J=6.0 Hz, 2H). MS m/z: 468.15 [M+H]⁺.

### Example 913: 7-(1-(2,2-difluoroethyl)-1H-pyrazolo[3,4-b]pyrazin-6-yl)-2-(6-(trifluoromethyl)pyridin-3-yl)-2,7-diazaspiro[3.5]nonan-6-one

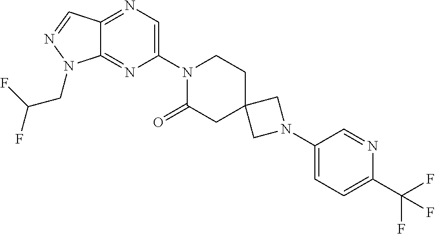 [SMILES: O=C1CC2(CCN1c1cnc3cnn(CC(F)F)c3n1)CN(c1ccc(C(F)(F)F)nc1)C2]

Step 1: 7-(1-(2,2-difluoroethyl)-1H-pyrazolo[3,4-b]pyrazin-6-yl)-2-(6-(trifluoromethyl)pyridin-3-yl)-2,7-diazaspiro[3.5]nonan-6-one: Followed the general procedure I using 5-chloro-2-(trifluoromethyl)pyridine (50 mg, 0.275 mmol, 1.0 equiv.) and 7-(1-(2,2-difluoroethyl)-1H-pyrazolo[3,4-b]pyrazin-6-yl)-2,7-diazaspiro[3.5]nonan-6-one (89 mg, 0.275 mmol, 1.0 equiv.) as the starting materials to give 7-(1-(2,2-difluoroethyl)-1H-pyrazolo[3,4-b]pyrazin-6-yl)-2-(6-(trifluoromethyl)pyridin-3-yl)-2,7-diazaspiro[3.5]nonan-6-one (27 mg, 21%) as a white solid. ¹H NMR (400 MHz, DMSO-d₆) δ 9.03 (s, 1H), 8.51 (s, 1H), 7.94 (d, J=2.8 Hz, 1H), 7.64 (d, J=8.6 Hz, 1H), 6.95 (dd, J=8.6, 2.7 Hz, 1H), 6.53 (tt, J=54.5, 3.6 Hz, 1H), 4.91 (td, J=15.2, 3.6 Hz, 2H), 4.04 (t, J=6.0 Hz, 2H), 3.98 (d, J=7.8 Hz, 2H), 3.90 (d, J=7.8 Hz, 2H), 2.99 (s, 2H), 2.29 (t, J=6.0 Hz, 2H). MS m/z: 468.05 [M+H]⁺.

### Example 914: 8-(1-(2,2-difluoroethyl)-1H-pyrazolo[3,4-b]pyrazin-6-yl)-2-(1-(2-(trifluoromethyl)pyrimidin-5-yl)ethyl)-2,8-diazaspiro[4.5]decan-3-one

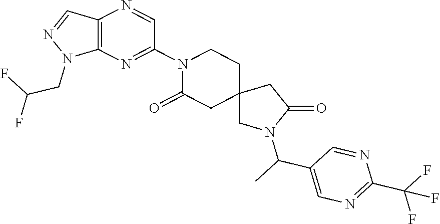 [SMILES: CC(c1cnc(C(F)(F)F)nc1)N1CC2(CCN(c3cnc4cnn(CC(F)F)c4n3)C(=O)C2)CC1=O]

Step 1: 1-(2-(trifluoromethyl)pyrimidin-5-yl)ethan-1-ol: A solution of 1-(2-(trifluoromethyl)pyrimidin-5-yl)ethan-1-one (1 g, 5.24 mmol, 1.0 equiv.) in THF (7.5 mL) was added LiBH₄ (483 mg, 21 mmol, 4.0 equiv.) in portions at 0° C. The resulting mixture was allowed to stir for 2 h at 0° C. under nitrogen atmosphere. The reaction was quenched with saturated NH₄Cl aq. (10 mL) at 0° C. The resulting mixture was extracted with EtOAc (3×10 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc=1/1 to afford 1-(2-(trifluoromethyl)pyrimidin-5-yl)ethan-1-ol (560 mg, 55%) as a colorless oil. MS m/z: 193 [M+H]⁺.

Step 2: 1-(2-(trifluoromethyl)pyrimidin-5-yl)ethyl methanesulfonate: Followed the general procedure R using 1-(2-(trifluoromethyl)pyrimidin-5-yl)ethan-1-ol (560 mg, 2.91 mmol, 1.0 equiv.) and methanesulfonic anhydride (545 mg, 3.11 mmol, 1.2 equiv.) as the starting materials to give 1-(2-(trifluoromethyl)pyrimidin-5-yl)ethyl methanesulfonate (460 mg, 58%) as a colorless oil. MS m/z: 271 [M+H]⁺.

Step 3: 8-(1-(2,2-difluoroethyl)-11-pyrazolo[3,4-b]pyrazin-6-yl)-2-(1-(2-(trifluoromethyl)pyrimidin-5-yl)ethyl)-2,8-diazaspiro[4.5]decan-3-one: Followed the general procedure E using 8-(1-(2,2-difluoroethyl)-1H-pyrazolo[3,4-b]pyrazin-6-yl)-2,8-diazaspiro[4.5]decan-3-one (150 mg, 0.443 mmol, 1.2 equiv.) and 1-(2-(trifluoromethyl)pyrimidin-5-yl)ethyl methanesulfonate (100 mg, 0.369 mmol, 1.0 equiv.) as the starting materials to give 8-(1-(2,2-difluoroethyl)-1H-pyrazolo[3,4-b]pyrazin-6-yl)-2-(1-(2-(trifluoromethyl)pyrimidin-5-yl)ethyl)-2,8-diazaspiro[4.5]decan-3-one (10.6 mg, 4%) as a white solid. ¹H NMR (400 MHz, DMSO-d₆) δ 9.02 (s, 2H), 8.47 (s, 1H), 8.12 (s, 1H), 6.43 (tt, J=54.9, 3.8 Hz, 1H), 5.42-5.32 (m, 1H), 4.75-4.62 (m, 2H), 3.96-3.78 (m, 2H), 3.71-3.61 (m, 2H), 3.34-3.32 (m, 1H), 3.11 (d, J=9.8 Hz, 1H), 2.44-2.27 (m, 2H), 1.66 (t, J=5.7 Hz, 2H), 1.58 (d, J=7.2 Hz, 5H). MS m/z: 511.2 [M+H]⁺.

### Example 915: 8-(1-(2,2-difluoroethyl)-1H-pyrazolo[3,4-b]pyrazin-6-yl)-2-((2-(trifluoromethyl)pyrimidin-4-yl)methyl)-2,8-diazaspiro[4.5]decan-3-one

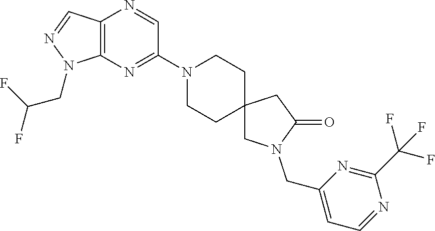 [SMILES: O=C1CC2(CCN(c3cnc4cnn(CC(F)F)c4n3)CC2)CN1Cc1ccnc(C(F)(F)F)n1]

Step 1: (2-(trifluoromethyl)pyrimidin-4-yl)methanol: Followed the general procedure X using 4-chloro-2-(trifluoromethyl)pyrimidine (200 mg, 1.09 mmol, 1.0 equiv.) and 4-chloro-2-(trifluoromethyl)pyrimidine (700 mg, 2.18 mmol, 2.0 equiv.) as the starting materials to give (2-(trifluoromethyl)pyrimidin-4-yl)methanol (150 mg, 78%) as a white solid. MS m/z: 179 [M+H]⁺.

Step 2: (2-(trifluoromethyl)pyrimidin-4-yl)methyl methanesulfonate: Followed the general procedure R using (2-(trifluoromethyl)pyrimidin-4-yl)methanol (150 mg, 0.843 mmol, 1.0 equiv.) and MsCl (96 mg, 0.843 mmol, 1.0 equiv.) as the starting materials to give (2-(trifluoromethyl)pyrimidin-4-yl)methyl methanesulfonate (80 mg, 37%) as a white solid. MS m/z: 257 [M+H]⁺.

Step 3: tert-butyl 3-oxo-2-((2-(trifluoromethyl)pyrimidin-4-yl)methyl)-2,8-diazaspiro[4.5]decane-8-carboxylate: Followed the general procedure E using (2-(trifluoromethyl)pyrimidin-4-yl)methyl methanesulfonate (80 mg, 0.312 mmol, 1.0 equiv.) and tert-butyl 3-oxo-2,8-diazaspiro[4.5]decane-8-carboxylate (80 mg, 0.312 mmol, 1.0 equiv.) as the starting materials to give tert-butyl 3-oxo-2-((2-(trifluoromethyl)pyrimidin-4-yl)methyl)-2,8-diazaspiro[4.5]decane-8-carboxylate (100 mg, 78%) as a white solid. MS m/z: 415 [M+H]⁺.

Step 4: 2-((2-(trifluoromethyl)pyrimidin-4-yl)methyl)-2,8-diazaspiro[4.5]decan-3-one hydrochloride: Followed the general procedure B using tert-butyl 3-oxo-2-((2-(trifluoromethyl)pyrimidin-4-yl)methyl)-2,8-diazaspiro[4.5]decane-8-carboxylate (100 mg) as the starting material to give the crude product 2-((2-(trifluoromethyl)pyrimidin-4-yl)methyl)-2,8-diazaspiro[4.5]decan-3-one hydrochloride (60 mg). MS m/z: 315 [M+H]⁺.

Step 5: 1-(3,5-difluorobenzyl)-8-(1-(2,2-difluoroethyl)-1H-pyrazolo[3,4-b]pyrazin-6-yl)-3-(5-(trifluoromethyl)pyrazin-2-yl)-1,3,8-triazaspiro[4.5]decane-2,4-dione: Followed the general procedure I using 2-((2-(trifluoromethyl)pyrimidin-4-yl)methyl)-2,8-diazaspiro[4.5]decan-3-one hydrochloride (60 mg, 0.191 mmol, 1.0 equiv.) and 6-chloro-1-(2,2-difluoroethyl)-1H-pyrazolo[3,4-b]pyrazine (41 mg, 0.191 mmol, 1.0 equiv.) as the starting materials to give 8-(1-(2,2-difluoroethyl)-1H-pyrazolo[3,4-b]pyrazin-6-yl)-2-((2-(trifluoromethyl)pyrimidin-4-yl)methyl)-2,8-diazaspiro[4.5]decan-3-one (31 mg, 33%) as a light yellow solid. ¹H NMR (400 MHz, DMSO-d₆) δ 9.02 (d, J=5.2 Hz, 1H), 8.48 (s, 1H), 8.12 (s, 1H), 7.74 (d, J=5.2 Hz, 1H), 6.43 (tt, J=54.9, 3.8 Hz, 1H), 4.75-4.63 (m, 4H), 3.88-3.68 (m, 4H), 3.35 (s, 2H), 2.40 (s, 2H), 1.72 (t, J=5.6 Hz, 4H). MS m/z: 497.15 [M+H]⁺.

### Example 916: 8-(1-(2,2-difluoroethyl)-1H-pyrazolo[3,4-d]pyrimidin-6-yl)-2-(2-(trifluoromethyl)pyrimidin-5-yl)-2,8-diazaspiro[4.5]decan-3-one

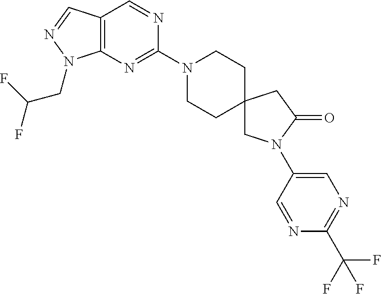 [SMILES: O=C1CC2(CCN(c3ncc4cnn(CC(F)F)c4n3)CC2)CN1c1cnc(C(F)(F)F)nc1]

Step 1: tert-butyl 3-oxo-2-(2-(trifluoromethyl)pyrimidin-5-yl)-2,8-diazaspiro[4.5]decane-8-carboxylate: Followed the general procedure Y using tert-butyl 3-oxo-2,8-diazaspiro[4.5]decane-8-carboxylate (150 mg, 0.588 mmol, 1.00 equiv.) and 5-bromo-2-(trifluoromethyl)pyrimidine (134 mg, 0.588 mmol, 1.00 equiv.) as the starting materials, Xphos Pd G3 (49.8 mg, 0.059 mmol, 0.10 equiv.) as the catalyst to give tert-butyl 3-oxo-2-(2-(trifluoromethyl)pyrimidin-5-yl)-2,8-diazaspiro[4.5]decane-8-carboxylate (150 mg, 63.6%) as a light yellow oil. MS m/z: 401 [M+H]⁺.

Step 2: 2-(2-(trifluoromethyl)pyrimidin-5-yl)-2,8-diazaspiro[4.5]decan-3-one hydrochloride: Followed the general procedure B using tert-butyl 3-oxo-2-(2-(trifluoromethyl)pyrimidin-5-yl)-2,8-diazaspiro[4.5]decane-8-carboxylate (150 mg, 0.374 mmol, 1.00 equiv.) as the starting material to give the crude product 2-(2-(trifluoromethyl)pyrimidin-5-yl)-2,8-diazaspiro[4.5]decan-3-one hydrochloride (110 mg) as a light yellow solid. MS m/z: 301 [M+H]⁺.

Step 3: 8-(1-(2,2-difluoroethyl)-1H-pyrazolo[3,4-d]pyrimidin-6-yl)-2-(2-(trifluoromethyl)pyrimidin-5-yl)-2,8-diazaspiro[4.5]decan-3-one: Followed the general procedure I using 2-(2-(trifluoromethyl)pyrimidin-5-yl)-2,8-diazaspiro[4.5]decan-3-one hydrochloride (110 mg, 0.365 mmol, 1.00 equiv.) and 6-chloro-1-(2,2-difluoroethyl)-1H-pyrazolo[3,4-d]pyrimidine (80.0 mg, 0.365 mmol, 1.00 equiv.) as the starting materials to give 8-(1-(2,2-difluoroethyl)-1H-pyrazolo[3,4-d]pyrimidin-6-yl)-2-(2-(trifluoromethyl)pyrimidin-5-yl)-2,8-diazaspiro[4.5]decan-3-one (72.8 mg, 41.2%) as a white solid. ¹H NMR (400 MHz, DMSO-d6) δ 9.36 (s, 2H), 8.92 (s, 1H), 8.08 (s, 1H), 6.44 (tt, 1H), 4.68 (td, 2H), 4.09-3.97 (m, 2H), 3.90 (s, 4H), 2.65 (s, 2H), 1.71 (t, 4H). MS m/z: 483.25 [M+H]⁺.

### Example 917: 8-(1-(2,2-difluoroethyl)-1H-pyrazolo[3,4-d]pyrimidin-6-yl)-2-((2-(trifluoromethyl)pyrimidin-5-yl)methyl)-2,8-diazaspiro[4.5]decan-3-one

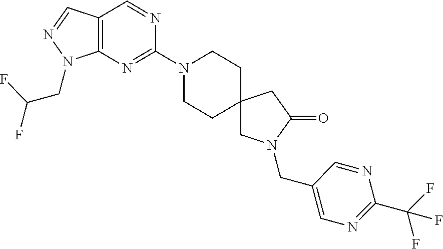 [SMILES: O=C1CC2(CCN(c3ncc4cnn(CC(F)F)c4n3)CC2)CN1Cc1cnc(C(F)(F)F)nc1]

Step 1: tert-butyl 3-oxo-2-((2-(trifluoromethyl)pyrimidin-5-yl)methyl)-2,8-diazaspiro[4.5]decane-8-carboxylate: Followed the general procedure E using tert-butyl 3-oxo-2,8-diazaspiro[4.5]decane-8-carboxylate (1 g, 3.94 mmol, 1.0 equiv.) and 5-(bromomethyl)-2-(trifluoromethyl)pyrimidine (1.04 g, 4.33 mmol, 1.1 equiv.) as the starting materials to give tert-butyl 3-oxo-2-((2-(trifluoromethyl)pyrimidin-5-yl)methyl)-2,8-diazaspiro[4.5]decane-8-carboxylate (400 mg, 25%) as a colorless oil. MS m/z: 415[M+H]⁺.

Step 2: 2-((2-(trifluoromethyl)pyrimidin-5-yl)methyl)-2,8-diazaspiro[4.5]decan-3-one hydrochloride: Followed the general procedure B using tert-butyl 3-oxo-2-((2-(trifluoromethyl)pyrimidin-5-yl)methyl)-2,8-diazaspiro[4.5]decane-8-carboxylate (200 mg) as the starting material to give the crude product 2-((2-(trifluoromethyl)pyrimidin-5-yl)methyl)-2,8-diazaspiro[4.5]decan-3-one hydrochloride (150 mg). MS m/z: 315 [M+H]⁺.

Step 3: 8-(1-(2,2-difluoroethyl pyrazolo[3,4-d]pyrimidin-6-yl)-2-(trifluoromethyl)pyrimidin-5-yl)methyl)-2,8-diazaspiro[4.5]decan-3-one: Followed the general procedure I using 2-((2-(trifluoromethyl)pyrimidin-5-yl)methyl)-2,8-diazaspiro[4.5]decan-3-one hydrochloride (80 mg, 0.254 mmol, 1.0 equiv.) and 6-chloro-1-(2,2-difluoroethyl)-1H-pyrazolo[3,4-d]pyrimidine (61 mg, 0.279 mmol, 1.1 equiv.) as the starting materials to give 8-(1-(2,2-difluoroethyl)-1H-pyrazolo[3,4-d]pyrimidin-6-yl)-2-((2-(trifluoromethyl)pyrimidin-5-yl)methyl)-2,8-diazaspiro[4.5]decan-3-one (37.9 mg, 30%) as a white solid. ¹H NMR (400 MHz, DMSO-d₆) δ 8.97 (s, 2H), 8.89 (s, 1H), 8.06 (s, 1H), 6.58-6.24 (m, 1H), 4.72-4.61 (m, 2H), 4.57 (s, 2H), 4.04-3.94 (m, 2H), 3.78-3.69 (m, 2H), 3.32 (s, 2H), 2.37 (s, 2H), 1.60 (s, 4H). MS m/z: 497.15 [M+H]⁺.

### Example 918: 8-(1-(2,2-difluoroethyl)-1H-pyrazolo[3,4-b]pyrazin-6-yl)-2-((6-(trifluoromethoxy)pyridin-3-yl)methyl)-2,8-diazaspiro[4.5]decan-3-one

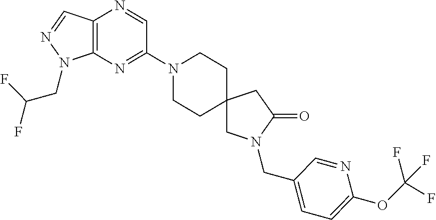 [SMILES: O=C1CC2(CCN(c3cnc4cnn(CC(F)F)c4n3)CC2)CN1Cc1ccc(OC(F)(F)F)nc1]

Step 1: methyl 6-(trifluoromethoxy)nicotinate: Followed the general procedure N using 5-bromo-2-(trifluoromethoxy)pyridine (300 mg, 1.24 mmol, 1.0 equiv) as the starting material, to give methyl 6-(trifluoromethoxy)nicotinate (160 mg, 57%) as a white solid. MS m/z: 222 [M+H]⁺.

Step 2: (6-(trifluoromethoxy)pyridin-3-yl)methanol: Followed the general procedure 0 using methyl 6-(trifluoromethoxy)nicotinate (160 mg, 0.724 mmol, 1.0 equiv) as the starting material to give (6-(trifluoromethoxy)pyridin-3-yl)methanol (120 mg, 85%) as a colorless oil. MS m/z: 194 [M+H]⁺.

Step 3: (6-(trifluoromethoxy)pyridin-3-yl)methyl methanesulfonate: Followed the general procedure R using (6-(trifluoromethoxy)pyridin-3-yl)methanol (120 mg, 0.621 mmol, 1.0 equiv) and Ms₂O (129 mg, 0.745 mmol, 1.2 equiv) as the starting materials to give (6-(trifluoromethoxy)pyridin-3-yl)methyl methanesulfonate (110 mg, 65%) as a white solid. MS m/z: 272 [M+H]⁺.

Step 4: 8-(1-(2,2-difluoroethyl)-1H-pyrazolo[3,4-b]pyrazin-6-yl)-2-((6-(trifluoromethoxy)pyridin-3-yl)methyl)-2,8-diazaspiro[4.5]decan-3-one: Followed the general procedure E using (6-(trifluoromethoxy)pyridin-3-yl)methyl methanesulfonate (89 mg, 0.328 mmol, 1.0 equiv) and 8-(1-(2,2-difluoroethyl)-1H-pyrazolo[3,4-b]pyrazin-6-yl)-2,8-diazaspiro[4.5]decan-3-one (110 mg, 0.328 mmol, 1.0 equiv) as the starting materials to give 8-(1-(2,2-difluoroethyl)-1H-pyrazolo[3,4-b]pyrazin-6-yl)-2-((6-(trifluoromethoxy)pyridin-3-yl)methyl)-2,8-diazaspiro[4.5]decan-3-one (51.8 mg, 30%) as a yellow solid. ¹H NMR (300 MHz, DMSO-d₆) δ 8.46 (s, 1H), 8.27 (d, J=2.4 Hz, 1H), 8.11 (s, 1H), 7.94-7.82 (m, 1H), 7.33-7.26 (m, 1H), 6.41 (tt, 1H), 4.65 (td, 2H), 4.46 (s, 2H), 3.93-3.73 (m, 2H), 3.73-3.59 (m, 2H), 3.18 (s, 2H), 2.37 (s, 2H), 1.67-1.50 (m, 4H). MS m/z: 512.10 [M+H]⁺.

### Example 919: 8-(1-(2,2-difluoroethyl)-1H-pyrazolo[3,4-b]pyrazin-6-yl)-2-((2-(difluoromethoxy)pyridin-4-yl)methyl)-2,8-diazaspiro[4.5]decan-3-one

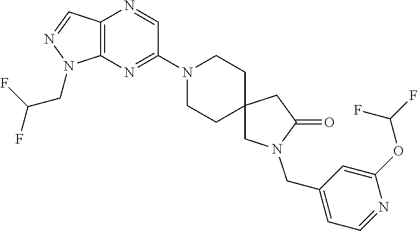 [SMILES: O=C1CC2(CCN(c3cnc4cnn(CC(F)F)c4n3)CC2)CN1Cc1ccnc(OC(F)F)c1]

Step 1: methyl 2-(difluoromethoxy)isonicotinate: Followed the general procedure N using 4-bromo-2-(difluoromethoxy)pyridine (400 mg, 0.45 mmol, 1.0 equiv.) as the starting material to give methyl 2-(difluoromethoxy)isonicotinate (320 mg, 71%) as a white solid. MS m/z: 203 [M+H]⁺.

Step 2: (2-(difluoromethoxy)pyridin-4-yl)methanol: Followed the general procedure O using methyl 2-(difluoromethoxy)isonicotinate (320 mg, 1.58 mmol, 1.0 equiv.) as the starting material to give (2-(difluoromethoxy)pyridin-4-yl)methanol (276 mg, 60%) as a white solid. MS m/z: 176 [M+H]⁺.

Step 3: (2-(difluoromethoxy)pyridin-4-yl)methyl methanesulfonate: Followed the general procedure R using (2-(difluoromethoxy)pyridin-4-yl)methanol (276 mg, 1.58 mmol, 1.0 equiv.) and MsCl (146 uL, 1.89 mmol, 1.2 equiv.) as the starting materials to give (2-(difluoromethoxy)pyridin-4-yl)methyl methanesulfonate (100 mg, 40%) as a white solid. MS m/z: 254 [M+H]⁺.

Step 4: 8-(1-(2,2-difluoroethyl)-1H-pyrazolo[3,4-b]pyrazin-6-yl)-2-((2-(difluoromethoxy)pyridin-4-yl)methyl)-2,8-diazaspiro[4.5]decan-3-one: Followed the general procedure E using (2-(difluoromethoxy)pyridin-4-yl)methyl methanesulfonate (60 mg, 0.237 mmol, 1.0 equiv.) and 8-(1-(2,2-difluoroethyl)-1H-pyrazolo[3,4-b]pyrazin-6-yl)-2,8-diazaspiro[4.5]decan-3-one (79.7 mg, 0.237 mmol, 1.0 equiv.) as the starting materials to give 8-(1-(2,2-difluoroethyl)-1H-pyrazolo[3,4-b]pyrazin-6-yl)-2-((2-(difluoromethoxy)pyridin-4-yl)methyl)-2,8-diazaspiro[4.5]decan-3-one (46.7 mg, 39%) as a white solid. ¹H NMR (400 MHz, DMSO-d₆) δ 8.47 (s, 1H), 8.24 (d, 1H), 8.12 (s, 1H), 7.72 (t, 1H), 7.20-7.07 (m, 1H), 6.93 (s, 1H), 6.44 (tt, 1H), 4.68 (td, 2H), 4.46 (s, 2H), 3.99-3.75 (m, 2H), 3.75-3.57 (m, 2H), 3.20 (s, 2H), 2.41 (s, 2H), 1.79-1.54 (m, 4H). MS m/z: 494.2 [M+H]⁺.

### Example 920: 8-(1-(2,2-difluoroethyl)-1H-pyrazolo[3,4-d]pyrimidin-6-yl)-2-(2-(2,2,2-trifluoroethoxy)pyrimidin-5-yl)-2,8-diazaspiro[4.5]decan-3-one

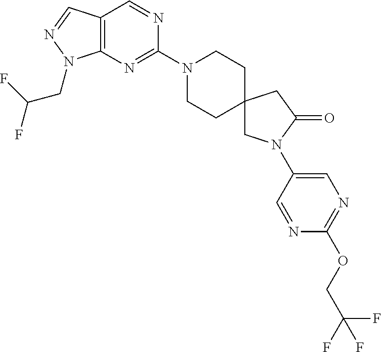 [SMILES: O=C1CC2(CCN(c3ncc4cnn(CC(F)F)c4n3)CC2)CN1c1cnc(OCC(F)(F)F)nc1]

Step 1: 5-bromo-2-(2,2,2-trifluoroethoxy)pyrimidine: Followed the general procedure E using 5-bromo-2-chloropyrimidine (300 mg, 1.55 mmol, 1.00 equiv.) and 2,2,2-trifluoroethan-1-ol (156 mg, 1.55 mmol, 1.00 equiv.) as the starting materials to give 5-bromo-2-(2,2,2-trifluoroethoxy)pyrimidine (200 mg, 50.1%) as a white solid. MS m/z: 258 [M+H]⁺.

Step 2: tert-butyl 3-oxo-2-(2-(2,2,2-trifluoroethoxy)pyrimidin-5-yl)-2,8-diazaspiro[4.5]decane-8-carboxylate: Followed the general procedure Y using 5-bromo-2-(2,2,2-trifluoroethoxy)pyrimidine (200 mg, 0.786 mmol, 1.00 equiv) and tert-butyl 3-oxo-2,8-diazaspiro[4.5]decane-8-carboxylate (202 mg, 0.786 mmol, 1.00 equiv) as the starting materials, EPhos Pd G4 (72.2 mg, 0.079 mmol, 0.10 equiv) as the catalyst to give tert-butyl 3-oxo-2-(2-(2,2,2-trifluoroethoxy)pyrimidin-5-yl)-2,8-diazaspiro[4.5]decane-8-carboxylate (85.0 mg, 25.1%) as a white solid. MS m/z: 431 [M+H]⁺.

Step 3: 2-(2-(2,2,2-trifluoroethoxy)pyrimidin-5-yl)-2,8-diazaspiro[4.5]decan-3-one hydrochloride: Followed the general procedure B using tert-butyl 3-oxo-2-(2-(2,2,2-trifluoroethoxy)pyrimidin-5-yl)-2,8-diazaspiro[4.5]decane-8-carboxylate (85.0 mg, 0.197 mmol, 1.00 equiv.) as the starting material to give the crude product 2-(2-(2,2,2-trifluoroethoxy)pyrimidin-5-yl)-2,8-diazaspiro[4.5]decan-3-one hydrochloride (60 mg). MS m/z: 331 [M+H]⁺.

Step 4: 8-(1-(2,2-difluoroethyl)-1H-pyrazolo[3,4-d]pyrimidin-6-yl)-2-(2-(2,2,2-trifluoroethoxy)pyrimidin-5-yl)-2,8-diazaspiro[4.5]decan-3-one: Followed the general procedure I using 2-(2-(2,2,2-trifluoroethoxy)pyrimidin-5-yl)-2,8-diazaspiro[4.5]decan-3-one hydrochloride (60.0 mg, 0.164 mmol, 1.00 equiv) and 6-chloro-1-(2,2-difluoroethyl)-1H-pyrazolo[3,4-b]pyrazine (35.8 mg, 0.164 mmol, 1.00 equiv) as the starting materials to give 8-(1-(2,2-difluoroethyl)-1H-pyrazolo[3,4-d]pyrimidin-6-yl)-2-(2-(2,2,2-trifluoroethoxy)pyrimidin-5-yl)-2,8-diazaspiro[4.5]decan-3-one (45.3 mg, 53.8%) as a white solid. ¹H NMR (300 MHz, DMSO-d₆) δ 8.98 (s, 2H), 8.52 (s, 1H), 8.15 (s, 1H), 6.45 (tt, 1H), 5.05 (q, 2H), 4.71 (td, 2H), 3.95-3.86 (m, 2H), 3.81 (s, 4H), 2.58 (s, 2H), 1.76 (t, 4H). MS m/z: 513.25 [M+H]⁺.

### Example 921: 8-(1-(2,2-difluoroethyl)-1H-pyrazolo[3,4-d]pyrimidin-6-yl)-2-(6-(2,2,2-trifluoroethoxy)pyridin-3-yl)-2,8-diazaspiro[4.5]decan-3-one

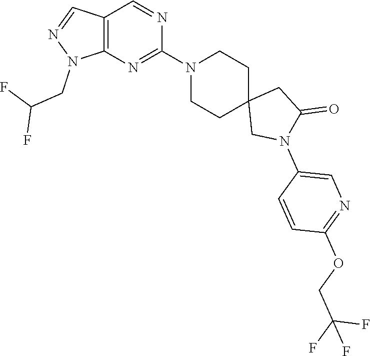 [SMILES: O=C1CC2(CCN(c3ncc4cnn(CC(F)F)c4n3)CC2)CN1c1ccc(OCC(F)(F)F)nc1]

Step 1: tert-butyl 3-oxo-2-(6-(2,2,2-trifluoroethoxy)pyridin-3-yl)-2,8-diazaspiro[4.5]decane-8-carboxylate: Followed the general procedure Y using tert-butyl 3-oxo-2,8-diazaspiro[4.5]decane-8-carboxylate (200 mg, 0.784 mmol, 1.00 equiv.) and 5-bromo-2-(2,2,2-trifluoroethoxy)pyridine (202 mg, 0.784 mmol, 1.00 equiv.) as the starting materials, Ephos Pd G4 (72.0 mg, 0.078 mmol, 0.100 eq), EPhos (41.9 mg, 0.078 mmol, 0.100 eq), as the catalyst to give tert-butyl 3-oxo-2-(6-(2,2,2-trifluoroethoxy)pyridin-3-yl)-2,8-diazaspiro[4.5]decane-8-carboxylate (200 mg, 59.3%) as a light yellow oil. MS m/z: 430 [M+H]⁺.

Step 2: 2-(6-(2,2,2-trifluoroethoxy)pyridin-3-yl)-2,8-diazaspiro[4.5]decan-3-one hydrochloride: Followed the general procedure B using tert-butyl 3-oxo-2-(6-(2,2,2-trifluoroethoxy)pyridin-3-yl)-2,8-diazaspiro[4.5]decane-8-carboxylate (200 mg, 0.465 mmol, 1.00 equiv.) as the starting material to give the crude product 2-(6-(2,2,2-trifluoroethoxy)pyridin-3-yl)-2,8-diazaspiro[4.5]decan-3-one hydrochloride (150 mg) as a light yellow solid. MS m/z: 330 [M+H]⁺

Step 3: 8-(1-(2,2-difluoroethyl)-1H-pyrazolo[3,4-d]pyrimidin-6-yl)-2-(6-(2,2,2-trifluoroethoxy)pyridin-3-yl)-2,8-diazaspiro[4.5]decan-3-one: Followed the general procedure I using 2-(6-(2,2,2-trifluoroethoxy)pyridin-3-yl)-2,8-diazaspiro[4.5]decan-3-one hydrochloride (150 mg, 0.455 mmol, 1.00 equiv.) and 6-chloro-1-(2,2-difluoroethyl)-1H-pyrazolo[3,4-d]pyrimidine (99.5 mg, 0.455 mmol, 1.00 equiv.) as the starting materials to give 8-(1-(2,2-difluoroethyl)-1H-pyrazolo[3,4-d]pyrimidin-6-yl)-2-(6-(2,2,2-trifluoroethoxy)pyridin-3-yl)-2,8-diazaspiro[4.5]decan-3-one (28 mg, 12.0%) as a white solid. ¹H NMR (400 MHz, DMSO-d₆) δ 8.91 (s, 1H), 8.40 (d, 1H), 8.19 (dd, 1H), 8.07 (s, 1H), 7.04 (d, 1H), 6.62-6.25 (m, 1H), 4.98 (q, 2H), 4.67 (td, 2H), 4.14-3.97 (m, 2H), 3.82 (dd, 6.2 Hz, 2H), 3.76 (s, 2H), 2.55 (s, 2H), 1.69 (t, 4H). MS m/z: 512.3 [M+H]⁺.

### Example 922: 8-(1-(2,2-difluoroethyl)-5-methyl-4-oxo-4,5-dihydro-1H-pyrazolo[3,4-d]pyrimidin-6-yl)-2-(4-(trifluoromethyl)pyridin-2-yl)-2,8-diazaspiro[4.5]decan-3-one

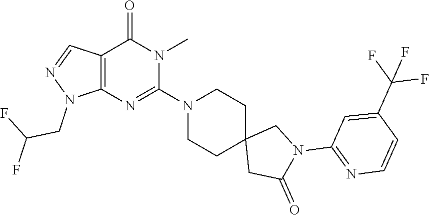 [SMILES: Cn1c(N2CCC3(CC2)CC(=O)N(c2cc(C(F)(F)F)ccn2)C3)nc2c(cnn2CC(F)F)c1=O]

Step 1: tert-butyl 3-oxo-2-(4-(trifluoromethyl)pyridin-2-yl)-2,8-diazaspiro[4.5]decane-8-carboxylate: Followed the general procedure E using tert-butyl 3-oxo-2,8-diazaspiro[4.5]decane-8-carboxylate (160 mg, 0.63 mmol, 1.0 equiv.) and 2-fluoro-4-(trifluoromethyl)pyridine (114 mg, 0.693 mmol, 1.1 equiv.) as the starting materials to give tert-butyl 3-oxo-2-(4-(trifluoromethyl)pyridin-2-yl)-2,8-diazaspiro[4.5]decane-8-carboxylate (110 mg, 44%) as a colorless oil. MS m/z: 400[M+H]⁺.

Step 2: 2-(4-(trifluoromethyl)pyridin-2-yl)-2,8-diazaspiro[4.5]decan-3-one hydrochloride: Followed the general procedure B using tert-butyl 3-oxo-2-(4-(trifluoromethyl)pyridin-2-yl)-2,8-diazaspiro[4.5]decane-8-carboxylate (110 mg) as the starting material to give the crude product 2-(4-(trifluoromethyl)pyridin-2-yl)-2,8-diazaspiro[4.5]decan-3-one hydrochloride (80 mg). MS m/z: 300 [M+H]⁺.

Step 3: 8-(1-(2,2-difluoroethyl)-5-methyl-4-oxo-4,5-dihydro-1H-pyrazolo[3,4-d]pyrimidin-6-yl)-2-(4-(trifluoromethyl)pyridin-2-yl)-2,8-diazaspiro[4.5]decan-3-one: Followed the general procedure I using 2-(4-(trifluoromethyl)pyridin-2-yl)-2,8-diazaspiro[4.5]decan-3-one hydrochloride (80 mg, 0.268 mmol, 1.0 equiv.) and 6-chloro-1-(2,2-difluoroethyl)-5-methyl-1,5-dihydro-4H-pyrazolo[3,4-d]pyrimidin-4-one (73 mg, 0.294 mmol, 1.1 equiv.) as the starting materials to give 8-(1-(2,2-difluoroethyl)-5-methyl-4-oxo-4,5-dihydro-1H-pyrazolo[3,4-d]pyrimidin-6-yl)-2-(4-(trifluoromethyl)pyridin-2-yl)-2,8-diazaspiro[4.5]decan-3-one (45.6 mg, 34%) as a white solid. ¹H NMR (400 MHz, DMSO-d₆) δ 8.70-8.62 (m, 2H), 8.02 (s, 1H), 7.52 (d, 1H), 6.60-6.26 (m, 1H), 4.64 (td, J=15.0, 4.0 Hz, 2H), 3.95 (s, 2H), 3.43 (s, 3H), 3.38-3.32 (m, 2H), 3.27-3.17 (m, 2H), 2.70 (s, 2H), 1.89-1.77 (m, 4H). MS m/z: 512.2 [M+H]⁺.

### Example 923: 8-(1-(2,2-difluoroethyl)-5-methyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one

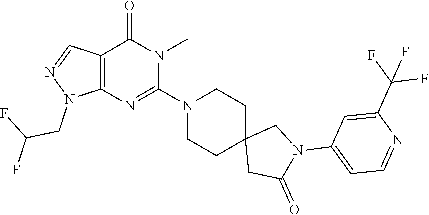 [SMILES: Cn1c(N2CCC3(CC2)CC(=O)N(c2ccnc(C(F)(F)F)c2)C3)nc2c(cnn2CC(F)F)c1=O]

Step 1: tert-butyl 3-oxo-2-(2-(trifluoromethyl)pyridin-4-yl)-2,8-diazaspiro[4.5]decane-8-carboxylate: Followed the general procedure I using tert-butyl 3-oxo-2,8-diazaspiro[4.5]decane-8-carboxylate (200 mg, 0.786 mmol, 1.0 equiv.) and 4-chloro-2-(trifluoromethyl)pyridine (171.31 mg, 0.943 mmol, 1.2 equiv.) as the starting materials to give tert-butyl 3-oxo-2-(2-(trifluoromethyl)pyridin-4-yl)-2,8-diazaspiro[4.5]decane-8-carboxylate (110 mg, 35%) as a white solid. MS m/z: 400 [M+H]⁺.

Step 2: 2-(2-(trifluoromethyl)pyridin-4-yl)-2,8-diazaspiro[4.5]decan-3-one: Followed the general procedure H using tert-butyl 3-oxo-2-(2-(trifluoromethyl)pyridin-4-yl)-2,8-diazaspiro[4.5]decane-8-carboxylate (110 mg) as the starting material to give the crude product 2-(2-(trifluoromethyl)pyridin-4-yl)-2,8-diazaspiro[4.5]decan-3-one (80 mg). MS m/z: 300 [M+H]⁺.

Step 3: 8-(1-(2,2-difluoroethyl)-5-methyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure I using 2-(2-(trifluoromethyl)pyridin-4-yl)-2,8-diazaspiro[4.5]decan-3-one (80 mg, 0.267 mmol, 1.0 equiv.) and 6-chloro-1-(2,2-difluoroethyl)-5-methyl-1,5-dihydro-4H-pyrazolo[3,4-d]pyrimidin-4-one (79 mg, 0.320 mmol, 1.2 equiv.) as the starting materials to give 8-(1-(2,2-difluoroethyl)-5-methyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one (69 mg, 50%) as a white solid. ¹H NMR (400 MHz, DMSO-d₆) δ 8.69 (d, J=5.6 Hz, 1H), 8.28 (d, J=2.1 Hz, 1H), 8.03 (s, 1H), 7.92 (dd, J=5.7, 2.0 Hz, 1H), 6.45 (tt, J=54.8, 3.8 Hz, 1H), 4.63 (td, J=14.8, 3.9 Hz, 2H), 3.87 (s, 2H), 3.43 (s, 3H), 3.29 (d, J=5.6 Hz, 4H), 2.66 (s, 2H), 1.81 (q, J=5.9, 5.4 Hz, 4H). MS m/z: 512.20 [M+H]⁺.

### Example 924: 6-[1-(2,2,2-trifluoroethyl)-1H-pyrazolo[3,4-b]pyrazin-6-yl]-2-[2-(trifluoromethyl)pyridin-4-yl]-2,6-diazaspiro[3.4]octane

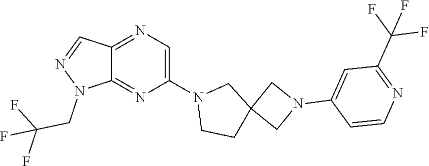 [SMILES: FC(F)(F)Cn1ncc2ncc(N3CCC4(CN(c5ccnc(C(F)(F)F)c5)C4)C3)nc21]

Followed the general procedure A to afford the desired product as a colorless oil (22 mg, 71%). MS m/z: 458 [M+H]⁺.

### Example 925: 8-(1-(2,2-difluoroethyl)-5-methyl-4-oxo-4,5-dihydro-1H-pyrazolo[3,4-d]pyrimidin-6-yl)-2-(6-(trifluoromethyl)pyridin-3-yl)-2,8-diazaspiro[4.5]decan-3-one

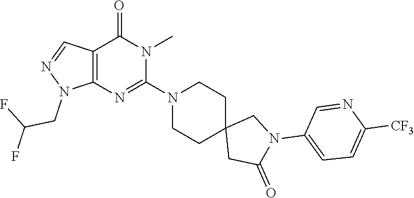 [SMILES: Cn1c(N2CCC3(CC2)CC(=O)N(c2ccc(C(F)(F)F)nc2)C3)nc2c(cnn2CC(F)F)c1=O]

Step 1: tert-butyl 3-oxo-2-(6-(trifluoromethyl)pyridin-3-yl)-2,8-diazaspiro[4.5]decane-8-carboxylate: Followed the general procedure E using tert-butyl 3-oxo-2,8-diazaspiro[4.5]decane-8-carboxylate (1 g, 3.93 mmol, 1.0 equiv.) and 5-fluoro-2-(trifluoromethyl)pyridine (710 mg, 4.32 mmol, 1.1 equiv.) as the starting materials to give tert-butyl 3-oxo-2-(6-(trifluoromethyl)pyridin-3-yl)-2,8-diazaspiro[4.5]decane-8-carboxylate (560 mg, 30%) as a white solid. MS m/z: 400 [M+H]⁺.

Step 2: 2-(6-(trifluoromethyl)pyridin-3-yl)-2,8-diazaspiro[4.5]decan-3-one hydrochloride: Followed the general procedure B using tert-butyl 3-oxo-2-(6-(trifluoromethyl)pyridin-3-yl)-2,8-diazaspiro[4.5]decane-8-carboxylate (560 mg) as the starting material to give the crude product 2-(6-(trifluoromethyl)pyridin-3-yl)-2,8-diazaspiro[4.5]decan-3-one hydrochloride (520 mg). MS m/z: 300 [M+H]⁺.

Step 3: 8-(1-(2,2-difluoroethyl)-5-methyl-4-oxo-4,5-dihydro-1H-pyrazolo[3,4-d]pyrimidin-6-yl)-2-(6-(trifluoromethyl)pyridin-3-yl)-2,8-diazaspiro[4.5]decan-3-one: Followed the general procedure I using 2-(6-(trifluoromethyl)pyridin-3-yl)-2,8-diazaspiro[4.5]decan-3-one hydrochloride (80 mg, 0.280 mmol, 1.0 equiv.) and 6-chloro-1-(2,2-difluoroethyl)-5-methyl-1,5-dihydro-4H-pyrazolo[3,4-d]pyrimidin-4-one (106 mg, 0.354 mmol, 1.1 equiv.) as the starting materials to give 8-(1-(2,2-difluoroethyl)-5-methyl-4-oxo-4,5-dihydro-1H-pyrazolo[3,4-d]pyrimidin-6-yl)-2-(6-(trifluoromethyl)pyridin-3-yl)-2,8-diazaspiro[4.5]decan-3-one (73.7 mg, 44%) as a white solid. ¹H NMR (400 MHz, DMSO-d₆) δ 9.07 (d, J=2.5 Hz, 1H), 8.59-8.32 (m, 1H), 7.99 (d, J=30.7 Hz, 1H), 7.93 (s, 1H), 6.70-6.23 (m, 1H), 4.63 (td, J=14.9, 3.9 Hz, 2H), 3.88 (s, 2H), 3.43 (s, 3H), 3.33-3.23 (m, 4H), 2.63 (s, 2H), 2.01-1.08 (m, 4H). MS m/z: 512.1 [M+H]⁺.

### Example 926: 8-(1-(2,2-difluoroethyl)-5-methyl-4-oxo-4,5-dihydro-1H-pyrazolo[3,4-d]pyrimidin-6-yl)-2-((2-(trifluoromethyl)pyrimidin-5-yl)methyl)-2,8-diazaspiro[4.5]decan-3-one

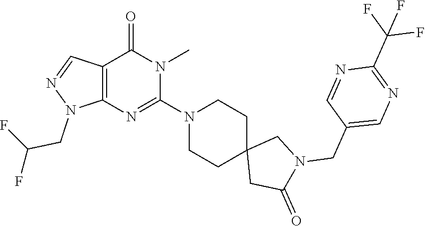 [SMILES: Cn1c(N2CCC3(CC2)CC(=O)N(Cc2cnc(C(F)(F)F)nc2)C3)nc2c(cnn2CC(F)F)c1=O]

Step 1: 8-(1-(2,2-difluoroethyl)-5-methyl-4-oxo-4,5-dihydro-1H-pyrazolo[3,4-d]pyrimidin-6-yl)-2-((2-(trifluoromethyl)pyrimidin-5-yl)methyl)-2,8-diazaspiro[4.5]decan-3-one: Followed the general procedure I using 2-((2-(trifluoromethyl)pyrimidin-5-yl)methyl)-2,8-diazaspiro[4.5]decan-3-one hydrochloride (70 mg, 0.223 mmol, 1.0 equiv.) and 6-chloro-1-(2,2-difluoroethyl)-5-methyl-1,5-dihydro-4H-pyrazolo[3,4-d]pyrimidin-4-one (60.9 mg, 0.245 mmol, 1.1 equiv.) as the starting materials to give 8-(1-(2,2-difluoroethyl)-5-methyl-4-oxo-4,5-dihydro-1H-pyrazolo[3,4-d]pyrimidin-6-yl)-2-((2-(trifluoromethyl)pyrimidin-5-yl)methyl)-2,8-diazaspiro[4.5]decan-3-one (55.5 mg, 40%) as a white solid. ¹H NMR (300 MHz, DMSO-d₆) δ 8.97 (s, 2H), 8.01 (s, 1H), 6.63-6.22 (m, 1H), 4.59 (d, J=15.5 Hz, 4H), 3.40 (s, 3H), 3.27 (s, 2H), 3.23-3.18 (m, 4H), 2.36 (s, 2H), 1.72 (t, J=5.4 Hz, 4H). MS m/z: 527.2 [M+H]⁺.

### Example 927: 8-(1-(2,2-difluoroethyl)-1H-pyrazolo[3,4-b]pyrazin-6-yl)-2-((5-(trifluoromethyl)pyrazin-2-yl)methyl)-2,8-diazaspiro[4.5]decan-1-one; Example 928: 8-(1-(2,2-difluoroethyl)-1H-pyrazolo[3,4-b]pyrazin-6-yl)-2-(3-methyl-6-(trifluoromethyl)pyrazin-2-yl)-2,8-diazaspiro[4.5]decan-1-one

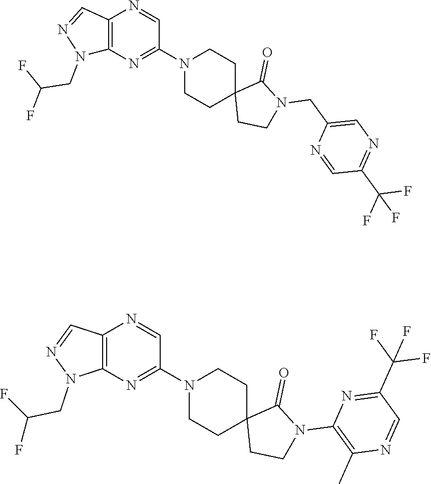

Step 1: 8-(1-(2,2-difluoroethyl)-1H-pyrazolo[3,4-b]pyrazin-6-yl)-2,8-diazaspiro[4.5]decan-1-one: Followed the general procedure I using 2,8-diazaspiro[4.5]decan-1-one (200 mg, 1.05 mmol, 1.0 equiv.) and 6-chloro-1-(2,2-difluoroethyl)-1H-pyrazolo[3,4-b]pyrazine (229 mg, 1.05 mmol, 1.00 equiv.) as the starting materials to give 8-(1-(2,2-difluoroethyl)-1H-pyrazolo[3,4-b]pyrazin-6-yl)-2,8-diazaspiro[4.5]decan-1-one (300 mg, 85%) as a white solid. MS m/z: 337 [M+H]⁺.

Step 2: (5-(trifluoromethyl)pyrazin-2-yl)methanol: Followed the general procedure X using 2-chloro-5-(trifluoromethyl)pyrazine (500 mg, 2.75 mmol, 1.0 equiv.) and (tributylstannyl)methanol (1.06 g, 3.30 mmol, 1.2 equiv.) as the starting materials to give (5-(trifluoromethyl)pyrazin-2-yl)methanol (300 mg, 61%) as a colorless oil. MS m/z: 179 [M+H]⁺.

Step 3: (5-(trifluoromethyl)pyrazin-2-yl)methyl methanesulfonate: Followed the general procedure R using (5-(trifluoromethyl)pyrazin-2-yl)methanol (300 mg, 1.68, 1.0 equiv.) and Ms₂O (323 mg, 1.85, 1.10 equiv.) as the starting materials to give (5-(trifluoromethyl)pyrazin-2-yl)methyl methanesulfonate (150 mg, 35%) as a colorless oil. MS m/z: 257 [M+H]⁺.

Step 4: 8-(1-(2,2-difluoroethyl)-1H-pyrazolo[3,4-b]pyrazin-6-yl)-2-((5-(trifluoromethyl)pyrazin-2-yl)methyl)-2,8-diazaspiro[4.5]decan-1-one: 8-(1-(2,2-difluoroethyl)-1H-pyrazolo[3,4-b]pyrazin-6-yl)-2-(3-methyl-6-(trifluoromethyl)pyrazin-2-yl)-2,8-diazaspiro[4.5]decan-1-one: To the solution of 8-(1-(2,2-difluoroethyl)-1H-pyrazolo[3,4-b]pyrazin-6-yl)-2,8-diazaspiro[4.5]decan-1-one (98.4 mg, 0.293 mmol, 1.0 equiv) and NaH (60% w/w, 35.2 mg, 0.879 mmol, 3.0 equiv.) in THF (2 mL) were added (5-(trifluoromethyl)pyrazin-2-yl)methyl methanesulfonate (150 mg, 0.586 mmol, 2.0 equiv.) in THF (1 mL) dropwise under N₂ atmosphere at 0° C. The result mixture was warmed to room temperature and stirred for 2 h. Desired product could be detected by LCMS. The mixture was quenched with saturated NH₄Cl aq., and extracted with EtOAc (2×10 mL). The combined organic layers was washed with brine, dried, filtered, evaporated, and purified by Combi-Flash to give 8-(1-(2,2-difluoroethyl)-1H-pyrazolo[3,4-b]pyrazin-6-yl)-2-((5-(trifluoromethyl)pyrazin-2-yl)methyl)-2,8-diazaspiro[4.5]decan-1-one (9.5 mg, 6.5%) and 8-(1-(2,2-difluoroethyl)-1H-pyrazolo[3,4-b]pyrazin-6-yl)-2-(3-methyl-6-(trifluoromethyl)pyrazin-2-yl)-2,8-diazaspiro[4.5]decan-1-one (20 mg, 13.8%) as white solids.

8-(1-(2,2-difluoroethyl)-1H-pyrazolo[3,4-b]pyrazin-6-yl)-2-((5-(trifluoromethyl)pyrazin-2-yl)methyl)-2,8-diazaspiro[4.5]decan-1-one: ¹H NMR (400 MHz, Methanol-d4) δ 8.98 (d, J=1.4 Hz, 1H), 8.72 (d, J=1.5 Hz, 1H), 8.37 (s, 1H), 8.00 (s, 1H), 6.26 (tt, J=55.4, 4.1 Hz, 1H), 4.77 (s, 2H), 4.68 (td, J=14.1, 4.1 Hz, 2H), 4.48 (dt, J=14.0, 4.4 Hz, 2H), 3.56 (t, J=6.9 Hz, 2H), 3.41 (ddd, J=14.0, 11.2, 3.1 Hz, 2H), 2.22 (t, J=7.0 Hz, 2H), 2.05-1.91 (m, 2H), 1.74-1.65 (m, 2H). MS m/z: 497.10 [M+H]⁺.

8-(1-(2,2-difluoroethyl)-1H-pyrazolo[3,4-b]pyrazin-6-yl)-2-(3-methyl-6-(trifluoromethyl)pyrazin-2-yl)-2,8-diazaspiro[4.5]decan-1-one: ¹H NMR (400 MHz, Chloroform-d) δ 8.75 (s, 1H), 8.26 (s, 1H), 8.06 (s, 1H), 6.22 (tt, J=55.6, 4.5 Hz, 1H), 4.67 (td, J=13.3, 4.5 Hz, 2H), 4.35 (dt, J=13.7, 4.8 Hz, 2H), 4.08 (t, J=7.0 Hz, 2H), 3.55 (ddd, J=13.5, 9.8, 3.4 Hz, 2H), 2.61 (s, 3H), 2.30 (t, J=7.0 Hz, 2H), 2.14 (ddd, J=13.7, 9.8, 4.0 Hz, 2H), 1.78 (dt, J=13.8, 4.7 Hz, 2H). MS m/z: 497.20 [M+H]⁺.

### Example 929: 1-(2,2-difluoroethyl)-6-(2-(2-methoxypyridin-4-yl)-2,6-diazaspiro[3.4]octan-6-yl)-1H-pyrazolo[3,4-b]pyrazine

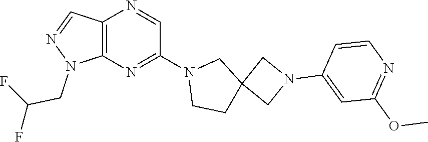 [SMILES: COc1cc(N2CC3(CCN(c4cnc5cnn(CC(F)F)c5n4)C3)C2)ccn1]

Step 1: 1-(2,2-difluoroethyl)-6-(2,6-diazaspiro[3.4]octan-6-yl)-1H-pyrazolo[3,4-b]pyrazine: Followed the general procedure H using tert-butyl 6-(1-(2,2-difluoroethyl)-1H-pyrazolo[3,4-b]pyrazin-6-yl)-2,6-diazaspiro[3.4]octane-2-carboxylate (100 mg) as the starting material to give the crude product 1-(2,2-difluoroethyl)-6-(2,6-diazaspiro[3.4]octan-6-yl)-1H-pyrazolo[3,4-b]pyrazine (100 mg). MS m/z: 309 [M+H]⁺.

Step 2: 1-(2,2-difluoroethyl)-6-(2-(2-methoxypyridin-4-yl)-2,6-diazaspiro[3.4]octan-6-yl)-1H-pyrazolo[3,4-b]pyrazine: Followed the general procedure I using 1-(2,2-difluoroethyl)-6-(2,6-diazaspiro[3.4]octan-6-yl)-1H-pyrazolo[3,4-b]pyrazine (150 mg, 0.59 mmol, 1.0 equiv.) and 4-fluoro-2-methoxypyridine (154 mg, 0.71 mmol, 1.2 equiv.) as the starting materials to give 1-(2,2-difluoroethyl)-6-(2-(2-methoxypyridin-4-yl)-2,6-diazaspiro[3.4]octan-6-yl)-1H-pyrazolo[3,4-b]pyrazine (130 mg, 50%) as a white solid. ¹H NMR (400 MHz, DMSO-d₆) δ 8.19-8.14 (s, 2H), 8.10 (s, 1H), 6.59 (s, 1H), 6.47-6.31 (m, 2H), 4.68 (td, J=14.8, 3.9 Hz, 2H), 4.16-4.04 (m, 4H), 3.86 (s, 2H), 3.67 (t, J=6.8 Hz, 2H), 2.32 (s, 5H). MS m/z: 402.1 [M+H]⁺.

### Example 930: 8-(1-(2,2-difluoroethyl)-1H-pyrazolo[3,4-b]pyrazin-6-yl)-2-((5-(trifluoromethyl)pyrazin-2-yl)methyl)-2,8-diazaspiro[4.5]decan-3-one

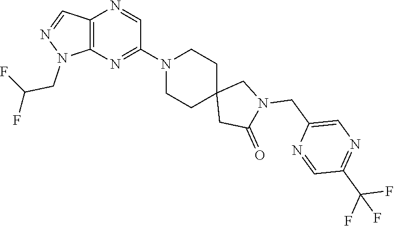 [SMILES: O=C1CC2(CCN(c3cnc4cnn(CC(F)F)c4n3)CC2)CN1Cc1cnc(C(F)(F)F)cn1]

Step 1: 8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decan-1-one: Followed the general procedure E using (5-(trifluoromethyl)pyrazin-2-yl)methyl methanesulfonate (100 mg, 0.39 mmol) and 8-(1-(2,2-difluoroethyl)-1H-pyrazolo[3,4-b]pyrazin-6-yl)-2,8-diazaspiro[4.5]decan-3-one (131 mg, 0.39 mmol, 1.0 equiv.) as the starting materials to give 8-(3-methyl-1-(oxetan-3-yl)-1H-pyrazolo[3,4-d]pyrimidin-6-yl)-2-(6-(trifluoromethyl)pyridin-3-yl)-2,8-diazaspiro[4.5]decan-1-one (17 mg, 8%) as a white solid. ¹H NMR (400 MHz, DMSO-d₆) δ 9.16 (s, 1H), 8.81 (s, 1H), 8.47 (d, J=2.2 Hz, 1H), 8.12 (d, J=2.2 Hz, 1H), 6.63-6.24 (m, 1H), 4.73-4.67 (m, 4H), 3.86-3.80 (m, 2H), 3.74-3.69 (m, 2H), 3.33-3.32 (m, 2H), 2.40-2.36 (m, 2H), 1.71-1.65 (m, 4H). MS m/z: 497.2 [M+H]⁺.

### Example 931: 3-methyl-1-(oxetan-3-yl)-6-(2-(2-(trifluoromethyl)pyridin-4-yl)-2,6-diazaspiro[3.4]octan-6-yl)-1H-pyrazolo[3,4-b]pyrazine

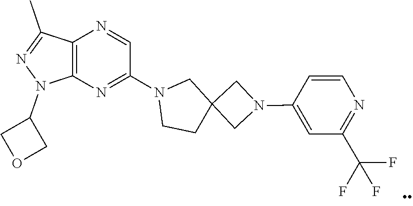 [SMILES: Cc1nn(C2COC2)c2nc(N3CCC4(CN(c5ccnc(C(F)(F)F)c5)C4)C3)cnc12]

Step 1: tert-butyl 2-(2-(trifluoromethyl)pyridin-4-yl)-2,6-diazaspiro[3.4]octane-6-carboxylate: Followed the general procedure Y using tert-butyl 2,6-diazaspiro[3.4]octane-6-carboxylate (500 mg, 2.36 mmol, 1.0 equiv.) and 4-bromo-2-(trifluoromethyl)pyridine (531 mg, 2.36 mmol, 1.0 equiv.) as the starting materials to give tert-butyl 2-(2-(trifluoromethyl)pyridin-4-yl)-2,6-diazaspiro[3.4]octane-6-carboxylate (750 mg, 89%) as a yellow solid. MS m/z: 358 [M+H]⁺.

Step 2: 2-(2-(trifluoromethyl)pyridin-4-yl)-2,6-diazaspiro[3.4]octane: Followed the general procedure H using tert-butyl 2-(2-(trifluoromethyl)pyridin-4-yl)-2,6-diazaspiro[3.4]octane-6-carboxylate (750 mg) as the starting material to give the crude product 2-(2-(trifluoromethyl)pyridin-4-yl)-2,6-diazaspiro[3.4]octane (750 mg). MS m/z: 258 [M+H]⁺.

Step 3: 3-methyl-1-(oxetan-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure I using 6-chloro-3-methyl-1-(oxetan-3-yl)-1H-pyrazolo[3,4-b]pyrazine (50 mg, 223 μmol, 1.0 equiv.) and 2-(2-(trifluoromethyl)pyridin-4-yl)-2,6-diazaspiro[3.4]octane (85.6 mg, 335 μmol, 1.5 equiv.) as the starting materials to give 3-methyl-1-(oxetan-3-yl)-6-(2-(2-(trifluoromethyl)pyridin-4-yl)-2,6-diazaspiro[3.4]octan-6-yl)-1H-pyrazolo[3,4-b]pyrazine (35.7 mg, 36%) as a white solid. ¹H NMR (400 MHz, DMSO-d₆) δ 8.25 (d, J=5.7 Hz, 1H), 8.01 (s, 1H), 6.76 (d, J=2.3 Hz, 1H), 6.58 (dd, J=5.7, 2.3 Hz, 1H), 5.81 (p, J=7.2 Hz, 1H), 5.10-5.00 (m, 2H), 4.97-4.89 (m, 2H), 4.04 (q, J=8.4 Hz, 4H), 3.84 (s, 2H), 3.65 (t, J=6.9 Hz, 2H), 2.46 (s, 3H), 2.30 (t, J=6.9 Hz, 2H). MS m/z: 446.15 [M+H]⁺.

### Example 932: 3-methyl-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine

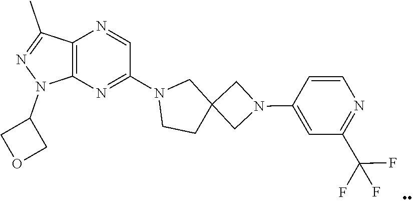 [SMILES: Cc1nn(C2COC2)c2nc(N3CCC4(CN(c5ccnc(C(F)(F)F)c5)C4)C3)cnc12]

Step 1: 3-methyl-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine: Followed the general procedure I using 6-chloro-3-methyl-1-(oxetan-3-yl)-1H-pyrazolo[3,4-d]pyrimidine (50 mg, 223 μmol, 1.0 equiv.) and 2-(2-(trifluoromethyl)pyridin-4-yl)-2,6-diazaspiro[3.4]octane (85.6 mg, 335 μmol, 1.5 equiv.) as the starting materials to give 3-methyl-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine (37.5 mg, 38%) as a white solid. ¹H NMR (400 MHz, DMSO-d₆) δ 8.87 (s, 1H), 8.25 (d, J=5.7 Hz, 1H), 6.75 (d, J=2.3 Hz, 1H), 6.57 (dd, J=5.7, 2.3 Hz, 1H), 5.85-5.73 (m, 1H), 5.06-4.98 (m, 2H), 4.97-4.89 (m, 2H), 4.08-3.96 (m, 4H), 3.82 (s, 2H), 3.65 (br, 2H), 2.47 (s, 3H), 2.31-2.22 (m, 2H). MS m/z: 446.15 [M+H]⁺.

### Example 933: (S)-1-(oxetan-2-ylmethyl)-6-(2-(2-(trifluoromethyl)pyridin-4-yl)-2,6-diazaspiro[3.4]octan-6-yl)-1H-pyrazolo[3,4-b]pyrazine

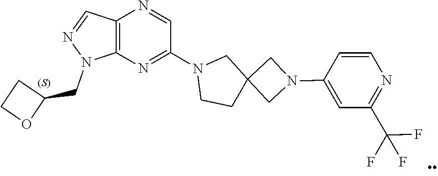

Step 1: (S)-6-chloro-1-(oxetan-2-ylmethyl)-1H-pyrazolo[3,4-b]pyrazine: Followed the general procedure C using (S)-oxetan-2-ylmethanol (200 mg, 2.27 mmol, 1.0 equiv.) and 6-chloro-1H-pyrazolo[3,4-b]pyrazine (526 mg, 3.41 mmol, 1.5 equiv.) as the starting materials to give (S)-6-chloro-1-(oxetan-2-ylmethyl)-1H-pyrazolo[3,4-b]pyrazine (300 mg, 59%) as a yellow solid. MS m/z: 225 [M+H]⁺.

Step 2: (S)-1-(oxetan-2-ylmethyl)-6-(2-(2-(trifluoromethyl)pyridin-4-yl)-2,6-diazaspiro[3.4]octan-6-yl)-1H-pyrazolo[3,4-b]pyrazine: Followed the general procedure I using (S)-6-chloro-1-(oxetan-2-ylmethyl)-1H-pyrazolo[3,4-b]pyrazine (100 mg, 445 μmol, 1.0 equiv.) and 2-(2-(trifluoromethyl)pyridin-4-yl)-2,6-diazaspiro[3.4]octane (172 mg, 667 μmol, 1.5 equiv.) as the starting materials to give (S)-1-(oxetan-2-ylmethyl)-6-(2-(2-(trifluoromethyl)pyridin-4-yl)-2,6-diazaspiro[3.4]octan-6-yl)-1H-pyrazolo[3,4-b]pyrazine (118 mg, 59%) as a yellow solid. ¹H NMR (400 MHz, DMSO-d₆) δ 8.25 (d, J=5.7 Hz, 1H), 8.09-8.04 (m, 2H), 6.76 (d, J=2.3 Hz, 1H), 6.58 (dd, J=5.7, 2.3 Hz, 1H), 5.16-5.05 (m, 1H), 4.56-4.40 (m, 3H), 4.39-4.31 (m, 1H), 4.11-4.00 (m, 4H), 3.85 (s, 2H), 3.67 (t, J=6.9 Hz, 2H), 2.71-2.63 (m, 1H), 2.62-2.54 (m, 1H), 2.31 (t, J=6.9 Hz, 2H). MS m/z: 446.3 [M+H]⁺.

### Example 934: (R)-1-(oxetan-2-ylmethyl)-6-(2-(2-(trifluoromethyl)pyridin-4-yl)-2,6-diazaspiro[3.4]octan-6-yl)-1H-pyrazolo[3,4-b]pyrazine

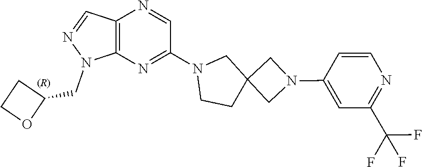

Step 1: (R)-6-chloro-1-(oxetan-2-ylmethyl)-1H-pyrazolo[3,4-b]pyrazine: Followed the general procedure C using (R)-6-chloro-1-(oxetan-2-ylmethyl)-1H-pyrazolo[3,4-b]pyrazine (60 mg, 0.68 mmol, 1.0 equiv.) and 6-chloro-1H-pyrazolo[3,4-b]pyrazine (148 mg, 0.68 mmol, 1.0 equiv.) as the starting materials to give (R)-6-chloro-1-(oxetan-2-ylmethyl)-1H-pyrazolo[3,4-b]pyrazine (100 mg, 65%) as a white solid. MS m/z: 225 [M+H]⁺.

Step 2: (R)-1-(oxetan-2-ylmethyl)-6-(2-(2-(trifluoromethyl)pyridin-4-yl)-2,6-diazaspiro[3.4]octan-6-yl)-1H-pyrazolo[3,4-b]pyrazine: Followed the general procedure I using (R)-6-chloro-1-(oxetan-2-ylmethyl)-1H-pyrazolo[3,4-b]pyrazine (60 mg, 0.27 mmol, 1.0 equiv.) and 2-(2-(trifluoromethyl)pyridin-4-yl)-2,6-diazaspiro[3.4]octane (89 mg, 0.35 mmol, 1.3 equiv.) as the starting materials to give (R)-1-(oxetan-2-ylmethyl)-6-(2-(2-(trifluoromethyl)pyridin-4-yl)-2,6-diazaspiro[3.4]octan-6-yl)-1H-pyrazolo[3,4-b]pyrazine (50 mg, 42%) as a white solid. ¹H NMR (400 MHz, DMSO-d₆) δ 8.25 (d, J=5.6 Hz, 1H), 8.06 (d, J=3.8 Hz, 2H), 6.76 (d, J=2.2 Hz, 1H), 6.58 (dd, J=5.7, 2.2 Hz, 1H), 5.10 (p, J=6.2 Hz, 1H), 4.56-4.41 (m, 3H), 4.39-4.32 (m, 1H), 4.13-3.99 (m, 4H), 3.85 (s, 2H), 3.67 (t, J=6.9 Hz, 2H), 2.72-2.54 (m, 2H), 2.31 (t, J=6.9 Hz, 2H). MS m/z: 446.15 [M+H]⁺.

### Example 935: 1-(2,2-difluoroethyl)-3-ethoxy-6-(2-(2-methyl-6-(trifluoromethyl)pyrimidin-4-yl)-2,6-diazaspiro[3.4]octan-6-yl)-1H-pyrazolo[3,4-b]pyrazine

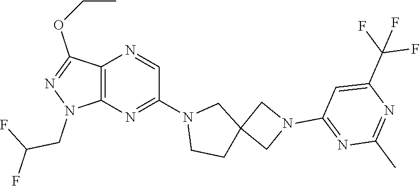 [SMILES: CCOc1nn(CC(F)F)c2nc(N3CCC4(CN(c5cc(C(F)(F)F)nc(C)n5)C4)C3)cnc12]

Step 1: 1-(2,2-difluoroethyl)-3-ethoxy-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure I using 2-(2-methyl-6-(trifluoromethyl)pyrimidin-4-yl)-2,6-diazaspiro[3.4]octane (110 mg, 0404 mmol, 1.0 equiv.) and 6-chloro-1-(2,2-difluoroethyl)-3-iodo-1H-pyrazolo[3,4-b]pyrazine (167 mg, 0.485 mmol, 1.5 equiv.) as the starting materials to give 1-(2,2-difluoroethyl)-3-ethoxy-6-(2-(2-methyl-6-(trifluoromethyl)pyrimidin-4-yl)-2,6-diazaspiro[3.4]octan-6-yl)-1H-pyrazolo[3,4-b]pyrazine (100 mg, 43%) as a colorless oil. MS m/z: 581 [M+H]⁺.

Step 2: 1-(2,2-difluoroethyl)-3-ethoxy-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure J using 1-(2,2-difluoroethyl)-3-ethoxy-6-(2-(2-methyl-6-(trifluoromethyl)pyrimidin-4-yl)-2,6-diazaspiro[3.4]octan-6-yl)-1H-pyrazolo[3,4-b]pyrazine (100 mg, 0.345 mmol, 2.0 equiv.) and EtOH (16 mg, 0.208 mmol, 1.1 equiv.) as the starting materials to give 1-(2,2-difluoroethyl)-3-ethoxy-6-(2-(2-methyl-6-(trifluoromethyl)pyrimidin-4-yl)-2,6-diazaspiro[3.4]octan-6-yl)-1H-pyrazolo[3,4-b]pyrazine (56.9 mg, 53%) as a white solid. ¹H NMR (400 MHz, DMSO-d₆) δ 7.94 (s, 1H), 6.66 (s, 1H), 6.37 (tt, J=55.0, 4.0 Hz, 1H), 4.49 (td, J=14.8, 3.9 Hz, 2H), 4.37 (q, J=7.0 Hz, 2H), 4.21-4.07 (m, 4H), 3.83 (s, 2H), 3.65 (t, J=6.8 Hz, 2H), 2.43 (s, 3H), 2.34-2.24 (m, 2H), 1.39 (t, J=7.0 Hz, 3H). MS m/z: 499.35 [M+H]⁺.

### Example 936: 1-(2,2-difluoroethyl)-3-methoxy-6-(2-(2-(trifluoromethyl)pyridin-4-yl)-2,6-diazaspiro[3.4]octan-6-yl)-1H-pyrazolo[3,4-b]pyrazine

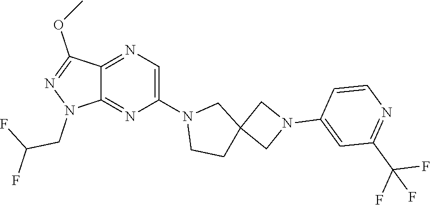 [SMILES: COc1nn(CC(F)F)c2nc(N3CCC4(CN(c5ccnc(C(F)(F)F)c5)C4)C3)cnc12]

Step 1: 6-chloro-3-iodo-1H-pyrazolo[3,4-b]pyrazine: Followed the general procedure V using 6-chloro-1H-pyrazolo[3,4-b]pyrazine (300 mg, 1.93 mmol, 1.0 equiv.) as the starting material, NIS (334 mg, 1.93 mol, 1.0 equiv.) as the reagent to give 6-chloro-3-iodo-1H-pyrazolo[3,4-b]pyrazine (480 mg, 88%) as a yellow solid. MS m/z: 281 [M+H]⁺.

Step 2: 3-iodo-6-(2-(2-(trifluoromethyl)pyridin-4-yl)-2,6-diazaspiro[3.4]octan-6-yl)-1H-pyrazolo[3,4-b]pyrazine: Followed the general procedure I using 6-chloro-3-iodo-1H-pyrazolo[3,4-b]pyrazine (480 mg, 1.71 mmol, 1.0 equiv.) and 2-(2-(trifluoromethyl)pyridin-4-yl)-2,6-diazaspiro[3.4]octane (440 mg, 1.71 mmol, 1.0 equiv.) as the starting materials to give 3-iodo-6-(2-(2-(trifluoromethyl)pyridin-4-yl)-2,6-diazaspiro[3.4]octan-6-yl)-1H-pyrazolo[3,4-b]pyrazine (145 mg, 17%) as a white solid. MS m/z: 502 [M+H]⁺.

Step 3: 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine: Followed the general procedure K using 3-iodo-6-(2-(2-(trifluoromethyl)pyridin-4-yl)-2,6-diazaspiro[3.4]octan-6-yl)-1H-pyrazolo[3,4-b]pyrazine (145 mg, 0.289 mmol, 1.0 equiv.) and 2,2-difluoroethyl trifluoromethanesulfonate (61.9 mg, 0.289 mol, 1.0 equiv.) as the starting materials to give 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine (100 mg, 61%) as a white solid. MS m/z: 566 [M+H]⁺.

Step 4: 1-(2,2-difluoroethyl)-3-methoxy-6-(2-(2-(trifluoromethyl)pyridin-4-yl)-2,6-diazaspiro[3.4]octan-6-yl)-1H-pyrazolo[3,4-b]pyrazine: Followed the general procedure J using 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine (100 mg, 0.177 mol) and methanol (100 mg, 0.167 mmol, 1.0 equiv.) as the starting materials to give 1-(2,2-difluoroethyl)-3-methoxy-6-(2-(2-(trifluoromethyl)pyridin-4-yl)-2,6-diazaspiro[3.4]octan-6-yl)-1H-pyrazolo[3,4-b]pyrazine (17.6 mg, 21%) as a white solid. ¹H NMR (300 MHz, DMSO-d₆) δ 8.25 (d, J=5.7 Hz, 1H), 7.96 (s, 1H), 6.77 (d, J=2.2 Hz, 1H), 6.62-6.10 (m, 2H), 4.50 (td, J=14.6, 3.9 Hz, 2H), 4.15-4.02 (m, 5H), 3.99 (s, 3H), 3.84 (s, 3H), 2.32-2.27 (m, 2H). MS m/z: 470.15 [M+H]⁺.

### Example 937: 1-(2,2-difluoroethyl)-3-ethoxy-6-(2-(2-(trifluoromethyl)pyridin-4-yl)-2,6-diazaspiro[3.4]octan-6-yl)-1H-pyrazolo[3,4-b]pyrazine

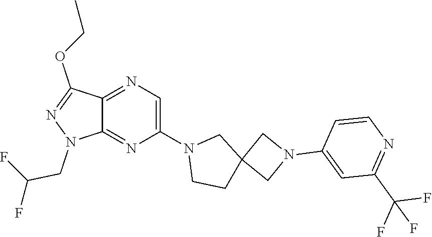 [SMILES: CCOc1nn(CC(F)F)c2nc(N3CCC4(CN(c5ccnc(C(F)(F)F)c5)C4)C3)cnc12]

1-(2,2-difluoroethyl)-3-ethoxy-6-(2-(2-(trifluoromethyl)pyridin-4-yl)-2,6-diazaspiro[3.4]octan-6-yl)-1H-pyrazolo[3,4-b]pyrazine: Followed the general procedure J using 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine (50 mg, 0.088 mmol, 1 equiv.) and ethanol (0.5 mL, 8.67 mmol, 48 equiv.) as the starting materials to give 1-(2,2-difluoroethyl)-3-ethoxy-6-(2-(2-(trifluoromethyl)pyridin-4-yl)-2,6-diazaspiro[3.4]octan-6-yl)-1H-pyrazolo[3,4-b]pyrazine (18.1 mg, 42%) as a white solid. ¹H NMR (300 MHz, DMSO-d₆) δ 8.25 (d, J=5.6 Hz, 1H), 7.95 (s, 1H), 6.76 (d, J=2.2 Hz, 1H), 6.61-6.14 (m, 2H), 4.56-4.41 (m, 2H), 4.41-4.31 (m, 2H), 4.10-3.97 (m, 4H), 3.84 (s, 2H), 3.65 (t, J=6.9 Hz, 2H), 2.30 (t, J=6.8 Hz, 2H), 1.39 (t, J=7.0 Hz, 3H). MS m/z: 484.15 [M+H]⁺.

### Example 938: 1-(2,2-difluoroethyl)-3-methoxy-6-(2-(2-(trifluoromethyl)pyridin-4-yl)-2,6-diazaspiro[3.4]octan-6-yl)-1H-pyrazolo[3,4-d]pyrimidine

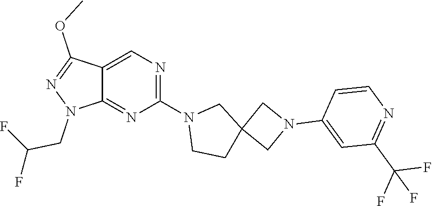 [SMILES: COc1nn(CC(F)F)c2nc(N3CCC4(CN(c5ccnc(C(F)(F)F)c5)C4)C3)ncc12]

Step 1: 1-(2,2-difluoroethyl)-3-methoxy-6-(2-(2-(trifluoromethyl)pyridin-4-yl)-2,6-diazaspiro[3.4]octan-6-yl)-1H-pyrazolo[3,4-d]pyrimidine: Followed the general procedure J using 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-d]pyrimidine (55 mg, 0.1 mmol, 1 equiv.) and MeOH (6.24 mg, 0.19 mmol, 2 equiv.) as the starting materials to give 1-(2,2-difluoroethyl)-3-methoxy-6-(2-(2-(trifluoromethyl)pyridin-4-yl)-2,6-diazaspiro[3.4]octan-6-yl)-1H-pyrazolo[3,4-d]pyrimidine (8.9 mg, 19%) as a white solid. ¹H NMR (400 MHz, DMSO-d₆) δ 8.76 (s, 1H), 8.24 (d, J=5.7 Hz, 1H), 6.75 (d, J=2.2 Hz, 1H), 6.63-6.49 (m, 1H), 6.41-6.22 (m, 1H), 4.58-4.41 (m, 2H), 4.04 (s, 2H), 4.00 (d, J=8.3 Hz, 2H), 3.96 (s, 3H), 3.82 (d, J=12.8 Hz, 2H), 3.64 (d, J=21.1 Hz, 2H), 2.26 (t, J=6.9 Hz, 2H). MS m/z: 470.15 [M+H]⁺.

### Example 939: 1-(2,2-difluoroethyl)-3-ethoxy-6-(2-(2-(trifluoromethyl)pyridin-4-yl)-2,6-diazaspiro[3.4]octan-6-yl)-1H-pyrazolo[3,4-d]pyrimidine

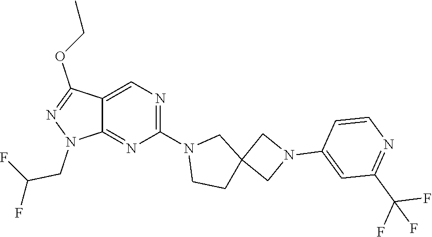 [SMILES: CCOc1nn(CC(F)F)c2nc(N3CCC4(CN(c5ccnc(C(F)(F)F)c5)C4)C3)ncc12]

Step 1: 3-iodo-6-(2-(2-(trifluoromethyl)pyridin-4-yl)-2,6-diazaspiro[3.4]octan-6-yl)-1H-pyrazolo[3,4-d]pyrimidine: Followed the general procedure I using 6-chloro-3-iodo-1H-pyrazolo[3,4-d]pyrimidine (150 mg, 0.535 mmol, 1.0 equiv.) and 2-(2-(trifluoromethyl)pyridin-4-yl)-2,6-diazaspiro[3.4]octane hydrochloride (138 mg, 0.535 mmol, 1.0 equiv.) as the starting materials to give 3-iodo-6-(2-(2-(trifluoromethyl)pyridin-4-yl)-2,6-diazaspiro[3.4]octan-6-yl)-1H-pyrazolo[3,4-d]pyrimidine (158 mg, 59%) as a yellow oil. MS m/z: 502 [M+H]⁺.

Step 2: 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-d]pyrimidine: Followed the general procedure K using 3-iodo-6-(2-(2-(trifluoromethyl)pyridin-4-yl)-2,6-diazaspiro[3.4]octan-6-yl)-1H-pyrazolo[3,4-d]pyrimidine (160 mg, 0.32 mmol, 1.0 equiv.) and 2,2-difluoroethyl trifluoromethanesulfonate (68.3 mg, 0.32 mmol, 1.0 equiv.) as the starting materials to give 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-d]pyrimidine (130 mg, 72%) as a colorless oil. MS m/z: 566 [M+H]⁺.

Step 3: 1-(2,2-difluoroethyl)-3-ethoxy-6-(2-(2-(trifluoromethyl)pridin-4-yl)-2,6-diazaspiro[3.4]octan-6-yl)-1H-pyrazolo[3,4-d]pyrimidine: Followed the general procedure J using 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-d]pyrimidine (55 mg, 0.097 mmol, 1.0 equiv.) and EtOH (8.96 mg, 0.194 mmol, 2.0 equiv.) as the starting materials to give 1-(2,2-difluoroethyl)-3-ethoxy-6-(2-(2-(trifluoromethyl)pyridin-4-yl)-2,6-diazaspiro[3.4]octan-6-yl)-1H-pyrazolo[3,4-d]pyrimidine (13.6 mg, 28%) as a white solid. ¹H NMR (400 MHz, DMSO-d₆) δ 8.75 (s, 1H), 8.24 (d, J=5.7 Hz, 1H), 6.75 (d, J=2.3 Hz, 1H), 6.60-6.55 (m, 1H), 6.53-6.22 (m, 1H), 4.54-4.41 (m, 2H), 4.37-4.30 (m, 2H), 4.01 (t, J=11.3 Hz, 4H), 3.88-3.78 (m, 2H), 3.64 (d, J=20.9 Hz, 2H), 2.26 (t, J=6.9 Hz, 2H), 1.38 (t, J=7.0 Hz, 3H). MS m/z: 484.2 [M+H]⁺.

### Example 940: 8-(1-(2,2-difluoroethyl)-3-methoxy-1H-pyrazolo[3,4-b]pyrazin-6-yl)-2-(2-(trifluoromethyl)pyridin-4-yl)-2,8-diazaspiro[4.5]decan-3-one

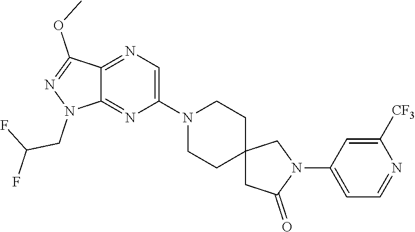 [SMILES: COc1nn(CC(F)F)c2nc(N3CCC4(CC3)CC(=O)N(c3ccnc(C(F)(F)F)c3)C4)cnc12]

Step 1: tert-butyl 3-oxo-2-(2-(trifluoromethyl)pyridin-4-yl)-2,8-diazaspiro[4.5]decane-8-carboxylate: Followed the general procedure Y using tert-butyl 3-oxo-2,8-diazaspiro[4.5]decane-8-carboxylate (350 mg, 1.376 mmol, 1 equiv) and 4-bromo-2-(trifluoromethyl)pyridine (373 mg, 1.651 mmol, 1.2 equiv) as the starting materials, RuPhos Pd G3 (115 mg, 0.138 mmol, 0.1 equiv) as the catalyst to give tert-butyl 3-oxo-2-(2-(trifluoromethyl)pyridin-4-yl)-2,8-diazaspiro[4.5]decane-8-carboxylate (370 mg, 67%) as a white solid. MS m/z: 400 [M+H]⁺.

Step 2: 2-(2-(trifluoromethyl)pyridin-4-yl)-2,8-diazaspiro[4.5]decan-3-one hydrochloride: Followed the general procedure B using tert-butyl 3-oxo-2-[2-(trifluoromethyl)pyridin-4-yl]-2,8-diazaspiro[4.5]decane-8-carboxylate (370 mg, 0.926 mmol, 1 equiv) as the starting material to give the crude product 2-(2-(trifluoromethyl)pyridin-4-yl)-2,8-diazaspiro[4.5]decan-3-one hydrochloride (100 mg). MS m/z: 300 [M+H]⁺.

Step 3: 8-(3-iodo-1-(tetrahydro-2H-pyran-3-yl)-1H-pyrazolo[3,4-b]pyrazin-6-yl)-2-(2-(trifluoromethyl)pyridin-4-yl)-2,8-diazaspiro[4.5]decan-3-one: Followed the general procedure I using 2-(2-(trifluoromethyl)pyridin-4-yl)-2,8-diazaspiro[4.5]decan-3-one hydrochloride (100 mg, 0.334 mmol, 1 equiv) and 6-chloro-3-iodo-1-(tetrahydro-2H-pyran-3-yl)-1H-pyrazolo[3,4-b]pyrazine (146 mg, 0.401 mmol, 1.2 equiv) as the starting materials to give 8-(3-iodo-1-(tetrahydro-2H-pyran-3-yl)-1H-pyrazolo[3,4-b]pyrazin-6-yl)-2-(2-(trifluoromethyl)pyridin-4-yl)-2,8-diazaspiro[4.5]decan-3-one (120 mg, 52%) as a white solid. MS m/z: 628 [M+H]⁺.

Step 4: 8-(3-methoxy-1-(tetrahydro-2H-pyran-3-yl)-1H-pyrazolo[3,4-b]pyrazin-6-yl)-2-(2-(trifluoromethyl)pyridin-4-yl)-2,8-diazaspiro[4.5]decan-3-one: Followed the general procedure J using 8-(3-iodo-1-(tetrahydro-2H-pyran-3-yl)-1H-pyrazolo[3,4-b]pyrazin-6-yl)-2-(2-(trifluoromethyl)pyridin-4-yl)-2,8-diazaspiro[4.5]decan-3-one (120 mg, 0.191 mmol, 1 equiv) and MeOH (18.4 mg, 0.573 mmol, 3 equiv) as the starting materials to give 8-(3-methoxy-1-(tetrahydro-2H-pyran-3-yl)-1H-pyrazolo[3,4-b]pyrazin-6-yl)-2-(2-(trifluoromethyl)pyridin-4-yl)-2,8-diazaspiro[4.5]decan-3-one (90 mg, 88%) as a white solid. MS m/z: 532 [M+H]⁺.

Step 5: 8-(3-methoxy-1H-pyrazolo[3,4-b]pyrazin-6-yl)-2-(2-(trifluoromethyl)pyridin-4-yl)-2,8-diazaspiro[4.5]decan-3-one: Followed the general procedure M using 8-(3-methoxy-1-(tetrahydro-2H-pyran-3-yl)-1H-pyrazolo[3,4-b]pyrazin-6-yl)-2-(2-(trifluoromethyl)pyridin-4-yl)-2,8-diazaspiro[4.5]decan-3-one (44 mg, 0.083 mmol, 1 equiv) as the starting material to give 8-(3-methoxy-1H-pyrazolo[3,4-b]pyrazin-6-yl)-2-(2-(trifluoromethyl)pyridin-4-yl)-2,8-diazaspiro[4.5]decan-3-one (35 mg, 97%) as a white solid. MS m/z: 447.4 [M+H]⁺.

Step 6: 8-(1-(2,2-difluoroethyl)-3-methoxy-1H-pyrazolo[3,4-b]pyrazin-6-yl)-2-(2-(trifluoromethyl)pyridin-4-yl)-2,8-diazaspiro[4.5]decan-3-one: Followed the general procedure K using 8-(3-methoxy-1H-pyrazolo[3,4-b]pyrazin-6-yl)-2-(2-(trifluoromethyl)pyridin-4-yl)-2,8-diazaspiro[4.5]decan-3-one (35 mg, 0.078 mmol, 1 equiv) and 2,2-difluoroethyl trifluoromethanesulfonate (20.1 mg, 0.094 mmol, 1.2 equiv) as the starting materials to give 8-(1-(2,2-difluoroethyl)-3-methoxy-1H-pyrazolo[3,4-b]pyrazin-6-yl)-2-(2-(trifluoromethyl)pyridin-4-yl)-2,8-diazaspiro[4.5]decan-3-one (15 mg, 49%) as a white solid. ¹H NMR (400 MHz, DMSO-d₆) δ 8.69 (d, J=5.7 Hz, 1H), 8.36 (s, 1H), 8.27 (d, J=2.2 Hz, 1H), 7.88 (dd, J=4.2 Hz, 1H), 6.39 (tt, 1H), 4.52 (td, J=14.9, 4.0 Hz, 2H), 4.01-3.97 (m, 3H), 3.91-3.83 (m, 4H), 3.81-3.73 (m, 2H), 2.66 (s, 2H), 1.77-1.71 (m, 4H). MS m/z: 512.25 [M+H]⁺.

### Example 941: 8-(1-(2,2-difluoroethyl)-3-ethoxy-1H-pyrazolo[3,4-b]pyrazin-6-yl)-2-(2-(trifluoromethyl)pyridin-4-yl)-2,8-diazaspiro[4.5]decan-3-one

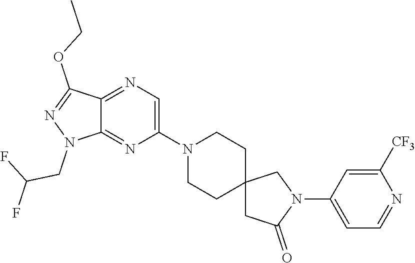 [SMILES: CCOc1nn(CC(F)F)c2nc(N3CCC4(CC3)CC(=O)N(c3ccnc(C(F)(F)F)c3)C4)cnc12]

Step 1: 8-(3-ethoxy-1-(tetrahydro-2H-pyran-2-yl)-1H-pyrazolo[3,4-b]pyrazin-6-yl)-2-(2-(trifluoromethyl)pyridin-4-yl)-2,8-diazaspiro[4.5]decan-3-one: Followed the general procedure J using 8-(3-iodo-1-(tetrahydro-2H-pyran-2-yl)-1H-pyrazolo[3,4-b]pyrazin-6-yl)-2-(2-(trifluoromethyl)pyridin-4-yl)-2,8-diazaspiro[4.5]decan-3-one (100 mg, 0.159 mmol, 1 equiv) and EtOH (6.13 mg, 0.191 mmol, 1.2 equiv) as the starting materials to give 8-(3-ethoxy-1-(tetrahydro-2H-pyran-2-yl)-1H-pyrazolo[3,4-b]pyrazin-6-yl)-2-(2-(trifluoromethyl)pyridin-4-yl)-2,8-diazaspiro[4.5]decan-3-one (100 mg, 88%) as a white solid. MS m/z: 546 [M+H]⁺.

Step 2: 8-(3-ethoxy-1H-pyrazolo[3,4-b]pyrazin-6-yl)-2-(2-(trifluoromethyl)pyridin-4-yl)-2,8-diazaspiro[4.5]decan-3-one: Followed the general procedure M using 8-(3-ethoxy-1-(tetrahydro-2H-pyran-2-yl)-1H-pyrazolo[3,4-b]pyrazin-6-yl)-2-(2-(trifluoromethyl)pyridin-4-yl)-2,8-diazaspiro[4.5]decan-3-one (100 mg, 0.183 mmol, 1 equiv) as the starting material to give 8-(3-ethoxy-1H-pyrazolo[3,4-b]pyrazin-6-yl)-2-(2-(trifluoromethyl)pyridin-4-yl)-2,8-diazaspiro[4.5]decan-3-one (85 mg, 97%) as a white solid. MS m/z: 462 [M+H]⁺.

Step 3: 8-(1-(2,2-difluoroethyl)-3-ethoxy-1H-pyrazolo[3,4-b]pyrazin-6-yl)-2-(2-(trifluoromethyl)pyridin-4-yl)-2,8-diazaspiro[4.5]decan-3-one: Followed the general procedure K using 8-(3-ethoxy-1H-pyrazolo[3,4-b]pyrazin-6-yl)-2-(2-(trifluoromethyl)pyridin-4-yl)-2,8-diazaspiro[4.5]decan-3-one (85 mg, 0.184 mmol, 1 equiv) and 2,2-difluoroethyl trifluoromethanesulfonate (47.3 mg, 0.221 mmol, 1.2 equiv) as the starting materials to give 8-(1-(2,2-difluoroethyl)-3-ethoxy-1H-pyrazolo[3,4-b]pyrazin-6-yl)-2-(2-(trifluoromethyl)pyridin-4-yl)-2,8-diazaspiro[4.5]decan-3-one (30 mg, 40%) as a white solid. ¹H NMR (400 MHz, DMSO-d₆) δ 8.69 (d, J=5.6 Hz, 1H), 8.35 (s, 1H), 8.27 (d, J=2.1 Hz, 1H), 7.89 (dd, J=5.8, 2.1 Hz, 1H), 6.36 (tt, J=55.0, 3.8 Hz, 1H), 4.51 (td, J=14.9, 3.9 Hz, 2H), 4.37 (q, J=7.0 Hz, 2H), 3.93-3.83 (m, 4H), 3.81-3.72 (m, 2H), 2.66 (s, 2H), 1.74 (d, J=6.5 Hz, 4H), 1.39 (t, J=7.0 Hz, 3H). MS m/z: 526.15 [M+H]⁺.

### Example 942: 8-(1-(2,2-difluoroethyl)-3-methoxy-1H-pyrazolo[3,4-b]pyrazin-6-yl)-2-(4-(trifluoromethyl)pyrimidin-2-yl)-2,8-diazaspiro[4.5]decan-3-one

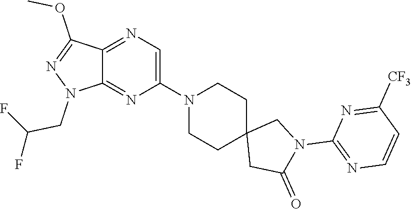 [SMILES: COc1nn(CC(F)F)c2nc(N3CCC4(CC3)CC(=O)N(c3nccc(C(F)(F)F)n3)C4)cnc12]

Step 1: 8-(3-iodo-1-(tetrahydro-2H-pyran-2-yl)-1H-pyrazolo[3,4-b]pyrazin-6-yl)-2,8-diazaspiro[4.5]decan-3-one: Followed the general procedure I using 2,8-diazaspiro[4.5]decan-3-one (400 mg, 2.58 mmol, 1.0 equiv.) and 6-chloro-3-iodo-1-(tetrahydro-2H-pyran-3-yl)-1H-pyrazolo[3,4-b]pyrazine (942 mg, 2.58 mmol, 1.0 equiv.) as the starting materials to give 8-(3-iodo-1-(tetrahydro-2H-pyran-2-yl)-1H-pyrazolo[3,4-b]pyrazin-6-yl)-2,8-diazaspiro[4.5]decan-3-one (750 mg, 60%) as a white solid. MS m/z: 483 [M+H]⁺.

Step 2: 8-(3-methoxy-1-(tetrahydro-2H-pyran-2-yl)-1H-pyrazolo[3,4-b]pyrazin-6-yl)-2,8-diazaspiro[4.5]decan-3-one: Followed the general procedure J using 8-(3-iodo-1-(tetrahydro-2H-pyran-2-yl)-1H-pyrazolo[3,4-b]pyrazin-6-yl)-2,8-diazaspiro[4.5]decan-3-one (370 mg, 0.766 mmol, 1.0 equiv.) and MeOH (26 mg, 0.766 mmol, 1.0 equiv.) as the starting materials to give 8-(3-methoxy-1-(tetrahydro-2H-pyran-2-yl)-1H-pyrazolo[3,4-b]pyrazin-6-yl)-2,8-diazaspiro[4.5]decan-3-one (40 mg, 14%) as a white solid. MS m/z: 387 [M+H]⁺.

Step 3: 8-(3-methoxy-1-(tetrahydro-2H-pyran-2-yl)-1H-pyrazolo[3,4-b]pyrazin-6-yl)-2-(4-(trifluoromethyl)pyrimidin-2-yl)-2,8-diazaspiro[4.5]decan-3-one: Followed the general procedure Y using 8-(3-methoxy-1-(tetrahydro-2H-pyran-2-yl)-1H-pyrazolo[3,4-b]pyrazin-6-yl)-2,8-diazaspiro[4.5]decan-3-one (40 mg, 0.103 mmol, 1.0 equiv.) and 2-chloro-4-(trifluoromethyl)pyrimidine (20 mg, 0.103 mmol, 1.0 equiv.) as the starting materials, XPhos Pd G3 (8.75 mg, 0.01 mmol, 0.01 equiv.) as the catalyst to give 8-(3-methoxy-1-(tetrahydro-2H-pyran-2-yl)-1H-pyrazolo[3,4-b]pyrazin-6-yl)-2-(4-(trifluoromethyl)pyrimidin-2-yl)-2,8-diazaspiro[4.5]decan-3-one (30 mg, 56%) as a white solid. MS m/z: 533 [M+H]⁺.

Step 4: 8-(3-methoxy-1H-pyrazolo[3,4-b]pyrazin-6-yl)-2-(4-(trifluoromethyl)pyrimidin-2-yl)-2,8-diazaspiro[4.5]decan-3-one: Followed the general procedure M using 8-(3-methoxy-1-(tetrahydro-2H-pyran-2-yl)-1H-pyrazolo[3,4-b]pyrazin-6-yl)-2-(4-(trifluoromethyl)pyrimidin-2-yl)-2,8-diazaspiro[4.5]decan-3-one (30 mg, 0.056 mmol, 1.0 equiv.) as the starting material to give 8-(3-methoxy-1H-pyrazolo[3,4-b]pyrazin-6-yl)-2-(4-(trifluoromethyl)pyrimidin-2-yl)-2,8-diazaspiro[4.5]decan-3-one (20 mg, 80%) as a white solid. MS m/z: 449 [M+H]⁺.

Step 5: 8-(1-(2,2-difluoroethyl)-3-methoxy-1H-pyrazolo[3,4-b]pyrazin-6-yl)-2-(4-(trifluoromethyl)pyrimidin-2-yl)-2,8-diazaspiro[4.5]decan-3-one: Followed the general procedure K using 8-(3-methoxy-1H-pyrazolo[3,4-b]pyrazin-6-yl)-2-(4-(trifluoromethyl)pyrimidin-2-yl)-2,8-diazaspiro[4.5]decan-3-one (20.0 mg, 0.045 mmol, 1.0 equiv.) and 2,2-difluoroethyl trifluoromethanesulfonate (28.7 mg, 0.134 μmol, 3.0 equiv.) as the starting materials to give (4.5 mg, 20%) as a white solid. ¹H NMR (300 MHz, Chloroform-d) δ 9.02 (d, J=4.9 Hz, 1H), 8.14 (s, 1H), 7.41 (d, J=4.9 Hz, 1H), 6.19 (tt, 1H), 4.50 (td, J=13.3, 4.5 Hz, 2H), 4.12 (s, 3H), 3.99 (d, J=22.3 Hz, 4H), 3.78-3.62 (m, 2H), 2.75 (s, 2H), 1.97-1.77 (m, 4H), 1.28 (s, 2H). MS m/z: 513.10 [M+H]⁺.

### Example 943: 3-methoxy-1-(oxetan-3-yl)-6-(2-(2-(trifluoromethyl)pyridin-4-yl)-2,6-diazaspiro[3.4]octan-6-yl)-1H-pyrazolo[3,4-b]pyrazine

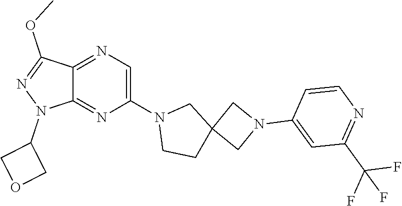 [SMILES: COc1nn(C2COC2)c2nc(N3CCC4(CN(c5ccnc(C(F)(F)F)c5)C4)C3)cnc12]

Step 1: 3-methoxy-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure J using 3-iodo-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine (210 mg, 0.358 mmol, 1.0 equiv.) and sodium 2-methylpropan-2-olate (103 mg, 1.07 mmol, 3.0 equiv.) as the starting materials to give 3-methoxy-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine (150 mg, 85%) as a yellow solid. MS m/z: 499 [M+H]⁺.

Step 2: 3-methoxy-6-(2-(2-(trifluoromethyl)pyridin-4-yl)-2,6-diazaspiro[3.4]octan-6-yl)-1H-pyrazolo[3,4-b]pyrazine: Followed the general procedure H using 3-methoxy-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine (150 mg) as the starting material to give the crude product 3-methoxy-6-(2-(2-(trifluoromethyl)pyridin-4-yl)-2,6-diazaspiro[3.4]octan-6-yl)-1H-pyrazolo[3,4-b]pyrazine (80 mg). MS m/z: 406 [M+H]⁺.

Step 3: 3-methoxy-1-(oxetan-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure K using 3-methoxy-6-(2-(2-(trifluoromethyl)pyridin-4-yl)-2,6-diazaspiro[3.4]octan-6-yl)-1H-pyrazolo[3,4-b]pyrazine (40 mg, 0.099 mmol, 1.0 equiv.) and oxetan-3-yl trifluoromethanesulfonate (30.5 mg, 0.149 mmol, 1.5 equiv.) as the starting materials to give 3-methoxy-1-(oxetan-3-yl)-6-(2-(2-(trifluoromethyl)pyridin-4-yl)-2,6-diazaspiro[3.4]octan-6-yl)-1H-pyrazolo[3,4-b]pyrazine (6.1 mg, 12%) as a yellow solid. ¹H NMR (300 MHz, Chloroform-d) δ 8.33 (d, J=5.2 Hz, 1H), 7.81 (s, 1H), 6.64 (s, 1H), 6.43 (s, 1H), 5.86-5.76 (m, 1H), 5.31 (t, J=6.5 Hz, 2H), 5.00-4.96 (m, 2H), 4.13 (d, J=10.5 Hz, 7H), 3.89 (s, 2H), 3.73 (t, J=6.7 Hz, 2H), 2.40 (t, J=6.8 Hz, 2H). MS m/z: 462.1 [M+H]⁺.

### Example 944: 3-ethoxy-1-(oxetan-3-yl)-6-(2-(2-(trifluoromethyl)pyridin-4-yl)-2,6-diazaspiro[3.4]octan-6-yl)-1H-pyrazolo[3,4-b]pyrazine

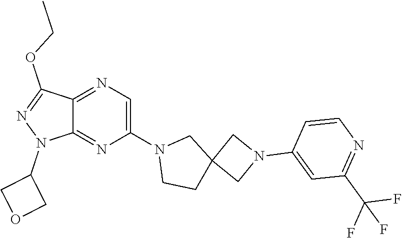 [SMILES: CCOc1nn(C2COC2)c2nc(N3CCC4(CN(c5ccnc(C(F)(F)F)c5)C4)C3)cnc12]

Step 1: 3-ethoxy-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure J using 3-iodo-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine (150 mg, 0.256 mmol, 1.0 equiv.) and sodium 2-methylpropan-2-olate (73.8 mg, 0.768 mmol, 3.0 equiv.) as the starting materials to give 3-ethoxy-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine (100 mg, 77%) as a yellow solid. MS m/z: 504 [M+H]⁺.

Step 2: 3-ethoxy-6-(2-(2-(trifluoromethyl)pyridin-4-yl)-2,6-diazaspiro[3.4]octan-6-yl)-1H-pyrazolo[3,4-b]pyrazine: Followed the general procedure H using 3-ethoxy-1-(tetrahydro-2H-pyran-3-yl)-6-(2-(2-(trifluoromethyl)pyridin-4-yl)-2,6-diazaspiro[3.4]octan-6-yl)-1H-pyrazolo[3,4-b]pyrazine (100 mg, 0.198 mmol, 1.0 equiv.) as the starting material to give the crude product 3-ethoxy-6-(2-(2-(trifluoromethyl)pyridin-4-yl)-2,6-diazaspiro[3.4]octan-6-yl)-1H-pyrazolo[3,4-b]pyrazine (80 mg). MS m/z: 420 [M+H]⁺.

Step 3: 3-ethoxy-1-(oxetan-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure K using 3-ethoxy-6-(2-(2-(trifluoromethyl)pyridin-4-yl)-2,6-diazaspiro[3.4]octan-6-yl)-1H-pyrazolo[3,4-b]pyrazine (40 mg, 0.095 mmol, 1.0 equiv.) and 3-iodooxetane (26.3 mg, 0.143 mmol, 1.5 equiv.) as the starting materials to give 3-ethoxy-1-(oxetan-3-yl)-6-(2-(2-(trifluoromethyl)pyridin-4-yl)-2,6-diazaspiro[3.4]octan-6-yl)-1H-pyrazolo[3,4-b]pyrazine (10.7 mg, 23%) as a white solid. ¹H NMR (300 MHz, DMSO-d₆) δ 8.24 (d, J=5.6 Hz, 1H), 7.92 (s, 1H), 6.74 (s, 1H), 6.57 (d, J=5.6 Hz, 1H), 5.74 (s, 1H), 5.01 (t, J=6.4 Hz, 2H), 4.90-4.85 (m, 2H), 4.43 (d, J=7.0 Hz, 2H), 4.06-3.99 (m, 4H), 3.82 (s, 2H), 3.64 (d, J=7.0 Hz, 2H), 2.30-2.25 (m, 2H), 1.40 (t, J=7.0 Hz, 3H). MS m/z: 476.1 [M+H]⁺.

### Example 945: 3-methoxy-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine

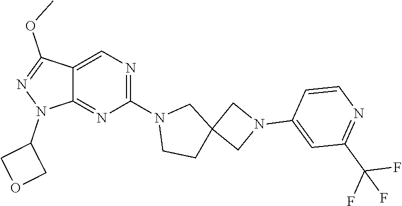 [SMILES: COc1nn(C2COC2)c2nc(N3CCC4(CN(c5ccnc(C(F)(F)F)c5)C4)C3)ncc12]

Step 1: 6-chloro-3-iodo-1-(tetrahydro-2H-pyran-2-yl)-1H-pyrazolo[3,4-d]pyrimidine: A solution of 6-chloro-3-iodo-1H-pyrazolo[3,4-d]pyrimidine (700 mg, 2.50 mmol, 1.0 equiv.), TsOH (43.0 mg, 0.25 mmol, 0.1 equiv.) and DHP (420 mg, 4.99 mmol, 2.0 equiv.) in DCM (3 mL) was stirred for 2 h at room temperature under air atmosphere. Desired product could be detected by LCMS. The resulting mixture was concentrated under reduced pressure. The residue was purified by silica gel column chromatography, eluted with PE/EA (1:1) to afford 6-chloro-3-iodo-1-(tetrahydro-2H-pyran-2-yl)-1H-pyrazolo[3,4-d]pyrimidine (510 mg, 56%) as a yellow oil. MS m/z: 365 [M+H]⁺.

Step 2: 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine: Followed the general procedure I using 6-chloro-3-iodo-1-(tetrahydro-2H-pyran-2-yl)-1H-pyrazolo[3,4-d]pyrimidine (500 mg, 1.37 mmol, 1.0 equiv.) and 2-(2-(trifluoromethyl)pyridin-4-yl)-2,6-diazaspiro[3.4]octane (353 mg, 1.37 mmol, 1.0 equiv.) as the starting materials to give 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (600 mg, 75%) as a yellow solid. MS m/z: 586 [M+H]⁺.

Step 3: 3-methoxy-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine: Followed the general procedure J using 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (350 mg, 0.598 mmol, 1.0 equiv.) and MeOH (38 mg, 1.2 mmol, 2.0 equiv.) as the starting materials to give 3-methoxy-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (230 mg, 78%) as a white solid. m/z: 490 [M+H]⁺.

Step 4: 3-methoxy-6-(2-(2-(trifluoromethyl)pyridin-4-yl)-2,6-diazaspiro[3.4]octan-6-yl)-1H-pyrazolo[3,4-d]pyrimidine: Followed the general procedure M using -methoxy-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (230 mg, 0.470 mmol, 1.0 equiv.) as the starting material to give 3-methoxy-6-(2-(2-(trifluoromethyl)pyridin-4-yl)-2,6-diazaspiro[3.4]octan-6-yl)-1H-pyrazolo[3,4-d]pyrimidine (170 mg, 89%) as a yellow oil. MS m/z: 406 [M+H]⁺.

Step 5: 3-methoxy-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine: Followed the general procedure I using 3-methoxy-6-(2-(2-(trifluoromethyl)871pyridine-4-yl)-2,6-diazaspiro[3.4]octan-6-yl)-1H-pyrazolo[3,4-d]pyrimidine (60 mg, 0.148 mmol, 1.0 equiv.) and 3-iodooxetane (29.9 mg, 0.163 mmol, 1.1 equiv.) as the starting materials to give 3-methoxy-1-(oxetan-3-yl)-6-(2-(2-(trifluoromethyl)871pyridine-4-yl)-2,6-diazaspiro[3.4]octan-6-yl)-1H-pyrazolo[3,4-d]pyrimidine (40.5 mg, 59%) as a white solid. ¹H NMR (300 MHz, Chloroform-d) 68.64 (s, 1H), 8.32 (d, J=5.1 Hz, 1H), 6.62 (s, 1H), 6.38 (s, 1H), 5.89-5.74 (m, 1H), 5.28 (t, J=6.5 Hz, 2H), 4.97 (dd, J=7.9, 6.3 Hz, 2H), 4.09 (s, 3H), 4.02 (q, J=7.9 Hz, 4H), 3.92 (s, 2H), 3.78 (s, 2H), 2.32 (t, J=6.8 Hz, 2H). MS m/z: 462.15 [M+H]⁺.

### Example 946: 3-ethoxy-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine

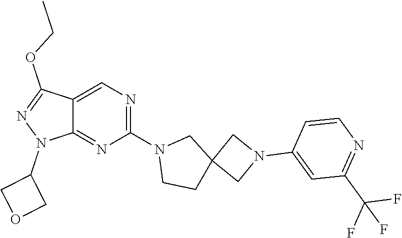 [SMILES: CCOc1nn(C2COC2)c2nc(N3CCC4(CN(c5ccnc(C(F)(F)F)c5)C4)C3)ncc12]

Step 1: 3-ethoxy-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine: Followed the general procedure J using 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (300 mg, 0.512 mmol, 1.0 equiv.) and EtOH (47.2 mg, 1.02 mmol, 2.0 equiv.) as the starting materials to give 3-ethoxy-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (180 mg, 34%) as white solid. MS m/z: 504 [M+H]⁺.

Step 2: 3-ethoxy-6-(2-(2-(trifluoromethyl)pyridin-4-yl)-2,6-diazaspiro[3.4]octan-6-yl)-1H-pyrazolo[3,4-d]pyrimidine: Followed the general procedure M using 3-ethoxy-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (220 mg, 0.437 mmol, 1 equiv.) as the starting material to give 3-ethoxy-6-(2-(2-(trifluoromethyl)pyridin-4-yl)-2,6-diazaspiro[3.4]octan-6-yl)-1H-pyrazolo[3,4-d]pyrimidine (150 mg, 82%) as a white solid. MS m/z: 420 [M+H]⁺.

Step 3: 3-ethoxy-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine: Followed the general procedure I using 3-ethoxy-6-(2-(2-(trifluoromethyl)pyridin-4-yl)-2,6-diazaspiro[3.4]octan-6-yl)-1H-pyrazolo[3,4-d]pyrimidine (75 mg, 0.179 mmol, 1.0 equiv.) and 3-iodooxetane (39.5 mg, 0.215 mmol, 1.2 equiv.) as the starting materials to give 3-ethoxy-1-(oxetan-3-yl)-6-(2-(2-(trifluoromethyl)pyridin-4-yl)-2,6-diazaspiro[3.4]octan-6-yl)-1H-pyrazolo[3,4-d]pyrimidine (26.8 mg, 31%) as a white solid. ¹H NMR (400 MHz, Chloroform-d) δ 8.65 (s, 1H), 8.32 (d, J=5.6 Hz, 1H), 6.62 (d, J=2.2 Hz, 1H), 6.37 (dd, J=5.8, 2.2 Hz, 1H), 5.88-5.72 (m, 1H), 5.26 (t, J=6.5 Hz, 2H), 4.97 (dd, J=7.9, 6.4 Hz, 2H), 4.47 (q, J=7.0 Hz, 2H), 4.07-3.97 (m, 4H), 3.92 (s, 2H), 3.79 (s, 2H), 2.32 (t, J=6.9 Hz, 2H), 1.48 (t, J=7.1 Hz, 3H). MS m/z: 476.15 [M+H]⁺.

### Example 947: 8-(1-(2,2-difluoroethyl)-3-ethoxy-1H-pyrazolo[3,4-b]pyrazin-6-yl)-2-(6-(trifluoromethyl)pyridin-3-yl)-2,8-diazaspiro[4.5]decan-3-one

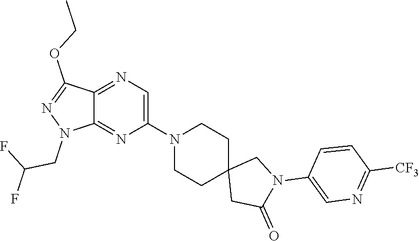 [SMILES: CCOc1nn(CC(F)F)c2nc(N3CCC4(CC3)CC(=O)N(c3ccc(C(F)(F)F)nc3)C4)cnc12]

Step 1: 8-(3-ethoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one: Followed the general procedure J using 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (240 mg, 0.383 mmol, 1.0 equiv.) and EtOH (35 mg, 0.761 mmol, 2.0 equiv.) as the starting materials to give 8-(3-ethoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (60 mg, 29%) as a white solid. MS m/z: 546 [M+H]⁺.

Step 2: 8-(3-ethoxy-1H-pyrazolo[3,4-b]pyrazin-6-yl)-2-(6-(trifluoromethyl)pyridin-3-yl)-2,8-diazaspiro[4.5]decan-3-one: Followed the general procedure M using 8-(3-ethoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (50 mg, 0.092 mmol, 1 equiv.) as the starting material to give 8-(3-ethoxy-1H-pyrazolo[3,4-b]pyrazin-6-yl)-2-(6-(trifluoromethyl)pyridin-3-yl)-2,8-diazaspiro[4.5]decan-3-one (40 mg, 95%) as a white solid. MS m/z: 462 [M+H]⁺.

Step 3: 8-(1-(2,2-difluoroethyl)-3-ethoxy-1H-pyrazolo[3,4-b]pyrazin-6-yl)-2-(6-(trifluoromethyl)pyridin-3-yl)-2,8-diazaspiro[4.5]decan-3-one: Followed the general procedure K using 8-(3-ethoxy-1H-pyrazolo[3,4-b]pyrazin-6-yl)-2-(6-(trifluoromethyl)pyridin-3-yl)-2,8-diazaspiro[4.5]decan-3-one (30 mg, 0.065 mmol, 1.0 equiv.) and 2,2-difluoroethyl trifluoromethanesulfonate (15.3 mg, 0.072 mmol, 1.1 equiv.) as the starting materials to give 8-(1-(2,2-difluoroethyl)-3-ethoxy-1H-pyrazolo[3,4-b]pyrazin-6-yl)-2-(6-(trifluoromethyl)pyridin-3-yl)-2,8-diazaspiro[4.5]decan-3-one (15.8 mg, 46%) as a white solid. ¹H NMR (300 MHz, DMSO-d₆) δ 9.05 (d, J=2.5 Hz, 1H), 8.46-8.27 (m, 2H), 7.94 (d, J=8.7 Hz, 1H), 6.41 (tt, 1H), 4.5 (td, 2H), 4.37 (q, J=7.0 Hz, 2H), 3.98-3.80 (m, 4H), 3.78-3.69 (m, 2H), 2.64 (s, 2H), 1.75 (d, J=5.9 Hz, 4H), 1.39 (t, J=7.0 Hz, 3H). MS m/z: 526.15[M+H]⁺.

### Example 948: 8-(1-(2,2-difluoroethyl)-3-methoxy-1H-pyrazolo[3,4-d]pyrimidin-6-yl)-2-(6-(trifluoromethyl)pyridin-3-yl)-2,8-diazaspiro[4.5]decan-3-one

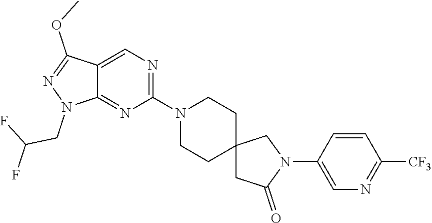 [SMILES: COc1nn(CC(F)F)c2nc(N3CCC4(CC3)CC(=O)N(c3ccc(C(F)(F)F)nc3)C4)ncc12]

Step 1: tert-butyl 3-oxo-2-(6-(trifluoromethyl)pyridin-3-yl)-2,8-diazaspiro[4.5]decane-8-carboxylate: Followed the general procedure I using tert-butyl 3-oxo-2,8-diazaspiro[4.5]decane-8-carboxylate (1000 mg, 3.92 mmol, 1.00 equiv.) and 5-fluoro-2-(trifluoromethyl)pyridine (781 mg, 4.71 mmol, 1.20 equiv.) as the starting materials to give tert-butyl 3-oxo-2-(6-(trifluoromethyl)pyridin-3-yl)-2,8-diazaspiro[4.5]decane-8-carboxylate (900 mg, 57.4%) as a light yellow oil. MS m/z: 400 [M+H]⁺.

Step 2: 2-(6-(trifluoromethyl)pyridin-3-yl)-2,8-diazaspiro[4.5]decan-3-one hydrochloride: Followed the general procedure B using tert-butyl 3-oxo-2-(6-(trifluoromethyl)pyridin-3-yl)-2,8-diazaspiro[4.5]decane-8-carboxylate (900 mg, 2.25 mmol, 1.00 equiv.) as the starting material to give the crude product 2-(6-(trifluoromethyl)pyridin-3-yl)-2,8-diazaspiro[4.5]decan-3-one hydrochloride (800 mg) as a light yellow solid. MS m/z: 300 [M+H]⁺

Step 3: 8-(3-iodo-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one: Followed the general procedure I using 2-(6-(trifluoromethyl)pyridin-3-yl)-2,8-diazaspiro[4.5]decan-3-one hydrochloride (400 mg, 1.33 mmol, 1.00 equiv.) and 6-chloro-3-iodo-1-(tetrahydro-2H-pyran-2-yl)-1H-pyrazolo[3,4-d]pyrimidine (583 mg, 1.60 mmol, 1.20 equiv.) as the starting materials to give 8-(3-iodo-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one (350 mg, 41.8%) as a light yellow oil. MS m/z: 628 [M+H]⁺.

Step 4: 8-(3-methoxy-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one: Followed the general procedure J using 8-(3-iodo-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one (350 mg, 0.557 mmol, 1.00 equiv.) and MeOH (0.2 mL) as the starting materials to give 8-(3-methoxy-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one (130 mg, 43.9%) as a white solid. MS m/z: 532 [M+H]⁺

Step 5: 8-(3-methoxy-1H-pyrazolo[3,4-d]pyrimidin-6-yl)-2-(6-(trifluoromethyl)pyridin-3-yl)-2,8-diazaspiro[4.5]decan-3-one: Followed the general procedure M using 8-(3-methoxy-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one (130 mg, 0.244 mmol, 1.00 equiv.) as the starting material to give 8-(3-methoxy-1H-pyrazolo[3,4-d]pyrimidin-6-yl)-2-(6-(trifluoromethyl)pyridin-3-yl)-2,8-diazaspiro[4.5]decan-3-one (70 mg, 63.9%) as a white solid. MS m/z: 448 [M+H]⁺

Step 6: 8-(1-(2,2-difluoroethyl)-3-methoxy-1H-pyrazolo[3,4-d]pyrimidin-6-yl)-2-(6-(trifluoromethyl)pyridin-3-yl)-2,8-diazaspiro[4.5]decan-3-one: Followed the general procedure K using 8-(3-methoxy-1H-pyrazolo[3,4-d]pyrimidin-6-yl)-2-(6-(trifluoromethyl)pyridin-3-yl)-2,8-diazaspiro[4.5]decan-3-one 40 mg, 0.089 mmol, 1.00 equiv.) and 2,2-difluoroethyl trifluoromethanesulfonate (21.1 mg, 0.098 mmol, 1.1 equiv.) as the starting materials to give 8-(1-(2,2-difluoroethyl)-3-methoxy-1H-pyrazolo[3,4-d]pyrimidin-6-yl)-2-(6-(trifluoromethyl)pyridin-3-yl)-2,8-diazaspiro[4.5]decan-3-one (19.0 mg, 41.6%) as a white solid. ¹H NMR (400 MHz, DMSO-d6) δ 9.05 (d, 1H), 8.75 (s, 1H), 8.39 (dd, 1H), 7.94 (d, 1H), 6.37 (tt, 1H), 4.49 (td, 2H), 4.12-3.98 (m, 2H), 3.96 (s, 3H), 3.90-3.72 (m, 4H), 2.63 (s, 2H), 1.69 (t, 4H). MS m/z: 512.15 [M+H]⁺.

### Example 949: 8-(1-(2,2-difluoroethyl)-3-ethoxy-1H-pyrazolo[3,4-d]pyrimidin-6-yl)-2-(6-(trifluoromethyl)pyridin-3-yl)-2,8-diazaspiro[4.5]decan-3-one

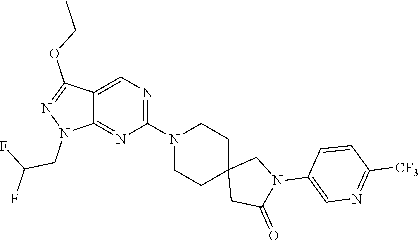 [SMILES: CCOc1nn(CC(F)F)c2nc(N3CCC4(CC3)CC(=O)N(c3ccc(C(F)(F)F)nc3)C4)ncc12]

Step 1: 8-(3-ethoxy-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one: Followed the general procedure J using 8-(3-iodo-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one (150 mg, 0.239 mmol, 1.00 equiv.) and EtOH (0.2 mL) as the starting materials to give 8-(3-ethoxy-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one (80 mg, 61.3%) as a white solid. MS m/z: 546 [M+H]⁺

Step 2: 8-(3-ethoxy-1H-pyrazolo[3,4-d]pyrimidin-6-yl)-2-(6-(trifluoromethyl)pyridin-3-yl)-2,8-diazaspiro[4.5]decan-3-one: Followed the general procedure M using 8-(3-ethoxy-1-(tetrahydro-2H-pyran-2-yl)-1H-pyrazolo[3,4-d]pyrimidin-6-yl)-2-(6-(trifluoromethyl)pyridin-3-yl)-2,8-diazaspiro[4.5]decan-3-one (80 mg, 0.147 mmol, 1.00 equiv.) as the starting material to give 8-(3-ethoxy-1H-pyrazolo[3,4-d]pyrimidin-6-yl)-2-(6-(trifluoromethyl)pyridin-3-yl)-2,8-diazaspiro[4.5]decan-3-one (40 mg, 59.2%) as a white solid. MS m/z: 462 [M+H]⁺.

Step 3: 8-(1-(2,2-difluoroethyl)-3-ethoxy-1H-pyrazolo[3,4-d]pyrimidin-6-yl)-2-(6-(trifluoromethyl)pyridin-3-yl)-2,8-diazaspiro[4.5]decan-3-one: Followed the general procedure K using 8-(3-ethoxy-1H-pyrazolo[3,4-d]pyrimidin-6-yl)-2-(6-(trifluoromethyl)pyridin-3-yl)-2,8-diazaspiro[4.5]decan-3-one 40 mg, 0.087 mmol, 1.00 equiv.) and 2,2-difluoroethyl trifluoromethanesulfonate (20.5 mg, 0.095 mmol, 1.1 equiv.) as the starting materials to give 8-(1-(2,2-difluoroethyl)-3-ethoxy-1H-pyrazolo[3,4-d]pyrimidin-6-yl)-2-(6-(trifluoromethyl)pyridin-3-yl)-2,8-diazaspiro[4.5]decan-3-one (22.5 mg, 49.4%) as a white solid. ¹H NMR (400 MHz, DMSO-d6) δ 9.05 (d, 1H), 8.68 (d, 1H), 8.39 (dd, 1H), 7.94 (d, 1H), 6.54-6.17 (m, 1H), 4.72-4.42 (m, 2H), 4.34 (q, 2H), 4.09-3.97 (m, 2H), 3.90-3.79 (m, 4H), 2.63 (d, 2H), 1.75-1.65 (m, 4H), 1.38 (t, 3H). MS m/z: 526.15 [M+H]⁺.

### Example 950: 8-(3-methoxy-1-(oxetan-3-yl)-1H-pyrazolo[3,4-b]pyrazin-6-yl)-2-(6-(trifluoromethyl)pyridin-3-yl)-2,8-diazaspiro[4.5]decan-3-one

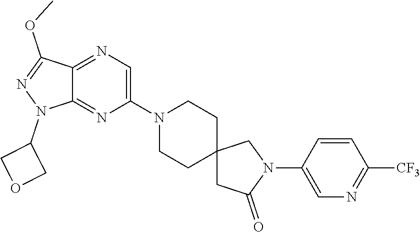 [SMILES: COc1nn(C2COC2)c2nc(N3CCC4(CC3)CC(=O)N(c3ccc(C(F)(F)F)nc3)C4)cnc12]

Step 1: 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one: Followed the general procedure J using 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (200 mg, 0.177 mmol, 1.0 equiv.) and MeOH (0.1 mL) as the starting materials to give 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (67 mg, 40%) as a white solid. MS m/z: 532 [M+H]⁺.

Step 2: 8-(3-methoxy-1H-pyrazolo[3,4-b]pyrazin-6-yl)-2-(6-(trifluoromethyl)pyridin-3-yl)-2,8-diazaspiro[4.5]decan-3-one: Followed the general procedure M using 8-(3-methoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (67 mg, 0.126 mmol, 1.0 equiv.) as the starting material to give 8-(3-methoxy-1H-pyrazolo[3,4-b]pyrazin-6-yl)-2-(6-(trifluoromethyl)pyridin-3-yl)-2,8-diazaspiro[4.5]decan-3-one (40 mg, 71%) as a yellow oil. MS m/z: 448 [M+H]⁺.

Step 3: 8-(3-methoxy-1-(oxetan-3-yl)-1H-pyrazolo[3,4-b]pyrazin-6-yl)-2-(6-(trifluoromethyl)pyridin-3-yl)-2,8-diazaspiro[4.5]decan-3-one: Followed the general procedure K using 8-(3-methoxy-1H-pyrazolo[3,4-b]pyrazin-6-yl)-2-(6-(trifluoromethyl)pyridin-3-yl)-2,8-diazaspiro[4.5]decan-3-one (42 mg, 0.094 mmol, 1.0 equiv.) and oxetan-3-yl trifluoromethanesulfonate (38.7 mg, 0.188 mmol, 2.0 equiv.) as the starting materials to give 8-(3-methoxy-1-(oxetan-3-yl)-1H-pyrazolo[3,4-b]pyrazin-6-yl)-2-(6-(trifluoromethyl)pyridin-3-yl)-2,8-diazaspiro[4.5]decan-3-one (20.7 mg, 43%) as a white solid. ¹H NMR (400 MHz, DMSO-d₆) δ 9.05 (d, J=2.5 Hz, 1H), 8.43-8.37 (m, 1H), 8.34 (s, 1H), 7.94 (d, J=8.7 Hz, 1H), 5.85-5.73 (m, 1H), 5.03 (t, J=6.4 Hz, 2H), 4.95-4.81 (m, 2H), 4.05 (s, 3H), 3.96-3.82 (m, 4H), 3.80-3.66 (m, 2H), 2.63 (s, 2H), 1.74 (t, J=5.7 Hz, 4H). MS m/z: 504.1 [M+H]⁺.

### Example 951: 8-(3-ethoxy-1-(oxetan-3-yl)-1H-pyrazolo[3,4-b]pyrazin-6-yl)-2-(6-(trifluoromethyl)pyridin-3-yl)-2,8-diazaspiro[4.5]decan-3-one

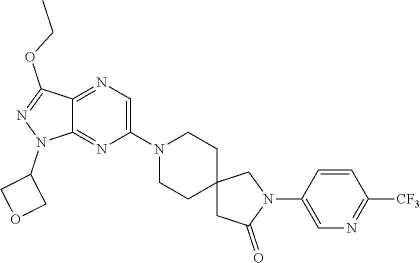 [SMILES: CCOc1nn(C2COC2)c2nc(N3CCC4(CC3)CC(=O)N(c3ccc(C(F)(F)F)nc3)C4)cnc12]

Step 1: tert-butyl 3-oxo-2-(6-(trifluoromethyl)pyridin-3-yl)-2,8-diazaspiro[4.5]decane-8-carboxylate: Followed the general procedure Y using tert-butyl 3-oxo-2,8-diazaspiro[4.5]decane-8-carboxylate (400 mg, 1.57 mmol, 1.0 equiv.) and 5-bromo-2-(trifluoromethyl)pyridine (427 mg, 1.9 mmol, 1.2 equiv.) as the starting materials to give tert-butyl 3-oxo-2-(6-(trifluoromethyl)pyridin-3-yl)-2,8-diazaspiro[4.5]decane-8-carboxylate (435 mg, 70%) as a white solid. MS m/z: 400 [M+H]⁺.

Step 2: 2-(6-(trifluoromethyl)pyridin-3-yl)-2,8-diazaspiro[4.5]decan-3-one hydrochloride: Followed the general procedure B using tert-butyl 3-oxo-2-(6-(trifluoromethyl)pyridin-3-yl)-2,8-diazaspiro[4.5]decane-8-carboxylate (435 mg) as the starting material to give the crude product 2-(6-(trifluoromethyl)pyridin-3-yl)-2,8-diazaspiro[4.5]decan-3-one hydrochloride (320 mg). MS m/z: 300 [M+H]⁺.

Step 3: 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one: Followed the general procedure I using 2-(6-(trifluoromethyl)pyridin-3-yl)-2,8-diazaspiro[4.5]decan-3-one hydrochloride (320 mg, 1.07 mmol, 1.0 equiv.) and 6-chloro-3-iodo-1-(tetrahydro-2H-pyran-2-yl)-1H-pyrazolo[3,4-b]pyrazine (477 mg, 1.28 mmol, 1.2 equiv.) as the starting materials to give 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (600 mg, 90%) as a white solid. MS m/z: 628 [M+H]⁺.

Step 4: 8-(3-ethoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one: Followed the general procedure J using 8-(3-iodo-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (300 mg, 414 μmol, 1.0 equiv.) and EtOH (0.1 mL) as the starting materials to give 8-(3-ethoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (150 mg, 58%) as a white solid. MS m/z: 546 [M+H]⁺.

Step 5: 8-(3-ethoxy-1H-pyrazolo[3,4-b]pyrazin-6-yl)-2-(6-(trifluoromethyl)pyridin-3-yl)-2,8-diazaspiro[4.5]decan-3-one: Followed the general procedure M using 8-(3-ethoxy-1-(tetrahydro-2H-pyran-2-yl)-1H-pyrazolo[3,4-b]pyrazin-6-yl)-2-(6-(trifluoromethyl)pyridin-3-yl)-2,8-diazaspiro[4.5]decan-3-one (150 mg, 414 μmol, 1.0 equiv.) as the starting material to give 8-(3-ethoxy-1H-pyrazolo[3,4-b]pyrazin-6-yl)-2-(6-(trifluoromethyl)pyridin-3-yl)-2,8-diazaspiro[4.5]decan-3-one (125 mg, 65%) as a white solid. MS m/z: 462 [M+H]⁺.

Step 6: 8-(3-ethoxy-1-(oxetan-3-yl)-1H-pyrazolo[3,4-b]pyrazin-6-yl)-2-(6-(trifluoromethyl)pyridin-3-yl)-2,8-diazaspiro[4.5]decan-3-one: Followed the general procedure K using 8-(3-ethoxy-1H-pyrazolo[3,4-b]pyrazin-6-yl)-2-(6-(trifluoromethyl)pyridin-3-yl)-2,8-diazaspiro[4.5]decan-3-one (125 mg, 271 μmol, 1.0 equiv.) and oxetan-3-yl trifluoromethanesulfonate (112 mg, 542 μmol, 2.0 equiv.) as the starting materials to give 8-(3-ethoxy-1-(oxetan-3-yl)-1H-pyrazolo[3,4-b]pyrazin-6-yl)-2-(6-(trifluoromethyl)pyridin-3-yl)-2,8-diazaspiro[4.5]decan-3-one (54.9 mg, 39%) as a white solid. ¹H NMR (400 MHz, DMSO-d₆) δ 9.05 (d, J=2.5 Hz, 1H), 8.40 (dd, J=8.7, 2.5 Hz, 1H), 8.33 (s, 1H), 7.95 (d, J=8.7 Hz, 1H), 5.85-5.72 (m, 1H), 5.01 (t, J=6.4 Hz, 2H), 4.89 (dd, J=7.9, 6.2 Hz, 2H), 4.45 (q, J=7.0 Hz, 2H), 3.89 (d, J=14.5 Hz, 4H), 3.78-3.67 (m, 2H), 2.63 (s, 2H), 1.74 (t, J=5.8 Hz, 4H), 1.42 (t, J=7.0 Hz, 3H). MS m/z: 518.19 [M+H]⁺.

### Example 952: 8-(3-methoxy-1-(oxetan-3-yl)-1H-pyrazolo[3,4-d]pyrimidin-6-yl)-2-(6-(trifluoromethyl)pyridin-3-yl)-2,8-diazaspiro[4.5]decan-3-one

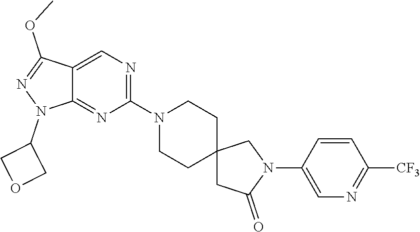 [SMILES: COc1nn(C2COC2)c2nc(N3CCC4(CC3)CC(=O)N(c3ccc(C(F)(F)F)nc3)C4)ncc12]

Step 1: 8-(3-methoxy-1-(oxetan-3-yl)-1H-pyrazolo[3,4-d]pyrimidin-6-yl)-2-(6-(trifluoromethyl)pyridin-3-yl)-2,8-diazaspiro[4.5]decan-3-one: Followed the general procedure K using 8-(3-methoxy-1H-pyrazolo[3,4-d]pyrimidin-6-yl)-2-(6-(trifluoromethyl)pyridin-3-yl)-2,8-diazaspiro[4.5]decan-3-one (40 mg, 0.089 mmol, 1.00 equiv.) and 3-iodooxetane (18.1 mg, 0.098 mmol, 1.10 equiv.) as the starting materials to give 8-(3-methoxy-1-(oxetan-3-yl)-1H-pyrazolo[3,4-d]pyrimidin-6-yl)-2-(6-(trifluoromethyl)pyridin-3-yl)-2,8-diazaspiro[4.5]decan-3-one (2.2 mg, 27.1%) as a white solid. ¹H NMR (400 MHz, DMSO-d6) δ 9.05 (d, 1H), 8.73 (s, 1H), 8.39 (dd, 1H), 7.94 (d, 1H), 5.90-5.69 (m, 1H), 5.01 (t, 2H), 4.90 (dd, 2H), 4.03 (s, 5H), 3.88-3.77 (m, 4H), 2.62 (s, 2H), 1.69 (t, 4H). MS m/z: 504.15 [M+H]⁺.

### Example 953: 8-(3-ethoxy-1-(oxetan-3-yl)-1H-pyrazolo[3,4-d]pyrimidin-6-yl)-2-(6-(trifluoromethyl)pyridin-3-yl)-2,8-diazaspiro[4.5]decan-3-one

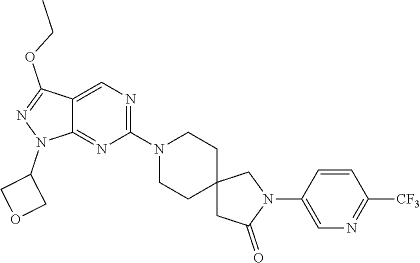 [SMILES: CCOc1nn(C2COC2)c2nc(N3CCC4(CC3)CC(=O)N(c3ccc(C(F)(F)F)nc3)C4)ncc12]

Step 1: 8-(3-ethoxy-1-(oxetan-3-yl)-1H-pyrazolo[3,4-d]pyrimidin-6-yl)-2-(6-(trifluoromethyl)pyridin-3-yl)-2,8-diazaspiro[4.5]decan-3-one: Followed the general procedure K using 8-(3-ethoxy-1H-pyrazolo[3,4-d]pyrimidin-6-yl)-2-(6-(trifluoromethyl)pyridin-3-yl)-2,8-diazaspiro[4.5]decan-3-one (40 mg, 0.087 mmol, 1.0 equiv.) and 3-iodooxetane (19.7 mg, 0.096 mmol, 1.1 equiv.) as the starting materials to give 8-(3-ethoxy-1-(oxetan-3-yl)-1H-pyrazolo[3,4-d]pyrimidin-6-yl)-2-(6-(trifluoromethyl)pyridin-3-yl)-2,8-diazaspiro[4.5]decan-3-one (17.2 mg, 38%) as a white solid. ¹H NMR (300 MHz, DMSO-d₆) δ 9.05 (d, J=2.5 Hz, 1H), 8.72 (s, 1H), 8.44-8.33 (m, 1H), 7.94 (d, J=8.5 Hz, 1H), 5.88-5.72 (m, 1H), 5.05-4.82 (m, 4H), 4.41 (q, J=7.0 Hz, 2H), 4.11-3.93 (m, 2H), 3.89-3.74 (m, 4H), 2.62 (s, 2H), 1.68 (t, J=5.7 Hz, 4H), 1.41 (t, J=7.0 Hz, 3H). MS m/z: 518.1 [M+H]⁺.

### Example 954: 8-(1-(2,2-difluoroethyl)-1H-pyrazolo[3,4-b]pyrazin-6-yl)-1-(2-fluoroethyl)-2-(2-(trifluoromethyl)pyridin-4-yl)-2,8-diazaspiro[4.5]decan-3-one

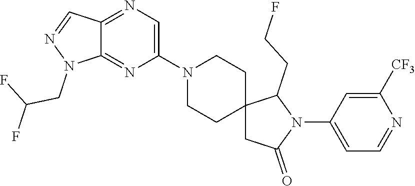 [SMILES: O=C1CC2(CCN(c3cnc4cnn(CC(F)F)c4n3)CC2)C(CCF)N1c1ccnc(C(F)(F)F)c1]

Step 1: 8-(1-(2,2-difluoroethyl)-1H-pyrazolo[3,4-b]pyrazin-6-yl)-1-(2-hydroxyethyl)-2-(2-(trifluoromethyl)pyridin-4-yl)-2,8-diazaspiro[4.5]decan-3-one: Followed the general procedure Q using 2-(8-(1-(2,2-difluoroethyl)-1H-pyrazolo[3,4-b]pyrazin-6-yl)-3-oxo-2-(2-(trifluoromethyl)pyridin-4-yl)-2,8-diazaspiro[4.5]decan-1-yl)acetaldehyde (60 mg, 0.114 mmol, 1.0 equiv.) as the starting material to give 8-(1-(2,2-difluoroethyl)-1H-pyrazolo[3,4-b]pyrazin-6-yl)-1-(2-hydroxyethyl)-2-(2-(trifluoromethyl)pyridin-4-yl)-2,8-diazaspiro[4.5]decan-3-one (45 mg, 75%) as a colorless oil. MS m/z: 526 [M+H]⁺.

Step 2: 8-(1-(2,2-difluoroethyl)-1H-pyrazolo[3,4-b]pyrazin-6-yl)-1-(2-fluoroethyl)-2-(2-(trifluoromethyl)pyridin-4-yl)-2,8-diazaspiro[4.5]decan-3-one: Followed the general procedure AD using 8-(1-(2,2-difluoroethyl)-1H-pyrazolo[3,4-b]pyrazin-6-yl)-1-(2-hydroxyethyl)-2-(2-(trifluoromethyl)pyridin-4-yl)-2,8-diazaspiro[4.5]decan-3-one (45 mg, 0.086 mmol, 1.0 equiv.) as the starting material to give 8-(1-(2,2-difluoroethyl)-1H-pyrazolo[3,4-b]pyrazin-6-yl)-1-(2-fluoroethyl)-2-(2-(trifluoromethyl)pyridin-4-yl)-2,8-diazaspiro[4.5]decan-3-one (20.9 mg, 46%) as a white solid. ¹H NMR (400 MHz, DMSO-d₆) δ 8.70 (d, J=5.6 Hz, 1H), 8.50 (s, 1H), 8.31 (d, J=2.1 Hz, 1H), 8.15 (s, 1H), 7.92 (dd, J=5.7, 2.2 Hz, 1H), 6.45 (tt, J=54.9, 3.7 Hz, 1H), 4.70 (td, J=15.0, 3.8 Hz, 2H), 4.55-4.17 (m, 5H), 3.58-3.47 (m, 1H), 3.36-3.33 (m, 1H), 2.86 (d, J=17.3 Hz, 1H), 2.71 (d, J=17.3 Hz, 1H), 2.33-2.16 (m, 1H), 2.03-1.88 (m, 1H), 1.84-1.70 (m, 3H), 1.69-1.59 (m, 1H). MS m/z: 528.05 [M+H]⁺.

### Example 955: 1-(2,2-difluoroethyl)-8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-3-one

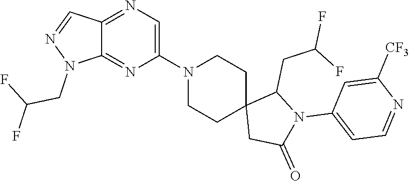 [SMILES: O=C1CC2(CCN(c3cnc4cnn(CC(F)F)c4n3)CC2)C(CC(F)F)N1c1ccnc(C(F)(F)F)c1]

Step 1: ethyl 2-(1-benzylpiperidin-4-ylidene)acetate: Followed the general procedure Z using 1-benzylpiperidin-4-one (10 g, 52.9 mmol, 1.0 equiv.) and ethyl 2-(diethoxyphosphoryl)acetate (11.9 mg, 52.9 mmol, 1.0 equiv.) as the starting materials to give ethyl 2-(1-benzylpiperidin-4-ylidene)acetate (10 g, 73%) as a colorless oil. MS m/z: 260 [M+H]⁺.

Step 2: ethyl 2-(4-(2-(1,3-dioxolan-2-yl)-1-nitroethyl)-1-benzylpiperidin-4-yl)acetate: A solution of ethyl 2-(1-benzylpiperidin-4-ylidene)acetate (3 g, 11.5 mmol, 1.0 equiv), TBAF (55.7 mL, 55.6 mmol, 5 equiv) and 2-(2-nitroethyl)-1,3-dioxolane (3.39 g, 23 mmol, 2.0 equiv) in THF (100 mL) was stirred for overnight at 60° C. The resulting mixture was concentrated under vacuum. The resulting mixture was extracted with EtOAc (200 mL). The combined organic layers were washed with water (3×40 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 100% gradient in 40 min; detector, UV 254 nm. This resulted in ethyl 2-(4-(2-(1,3-dioxolan-2-yl)-1-nitroethyl)-1-benzylpiperidin-4-yl)acetate (1.2 g, 25%) as a colorless oil. MS m/z: 407 [M+H]⁺.

Step 3: 1-((1,3-dioxolan-2-yl)methyl)-2,8-diazaspiro[4.5]decan-3-one: A solution of ethyl 2-(4-(2-(1,3-dioxolan-2-yl)-1-nitroethyl)-1-benzylpiperidin-4-yl)acetate (1.2 g, 2.95 mmol, 1.0 equiv.) and Pd/C (120 mg) in CF₃CH₂OH (10 mL) was stirred for overnight at 80° C. under hydrogen atmosphere. The resulting mixture was concentrated under vacuum. The crude product (0.65 g) was used in the next step directly without further purification. MS m/z: 241 [M+H]⁺.

Step 4: 1-((1,3-dioxolan-2-yl)methyl)-8-(1-(2,2-difluoroethyl)-1H-pyrazolo[3,4-b]pyrazin-6-yl)-2,8-diazaspiro[4.5]decan-3-one: Followed the general procedure I using 1-((1,3-dioxolan-2-yl)methyl)-2,8-diazaspiro[4.5]decan-3-one (0.65 g, 2.69 mmol, 1.0 equiv.) and 6-chloro-1-(2,2-difluoroethyl)-1H-pyrazolo[3,4-b]pyrazine (647 mg, 2.96 mmol, 1.1 equiv.) as the starting materials to give 1-((1,3-dioxolan-2-yl)methyl)-8-(1-(2,2-difluoroethyl)-1H-pyrazolo[3,4-b]pyrazin-6-yl)-2,8-diazaspiro[4.5]decan-3-one (700 mg, 61%) as a white solid. MS m/z: 423 [M+H]⁺.

Step 5: 1-((1,3-dioxolan-2-yl)methyl)-8-(1-(2,2-difluoroethyl)-1H-pyrazolo[3,4-b]pyrazin-6-yl)-2,8-diazaspiro[4.5]decan-3-one: Followed the general procedure Y using 1-((1,3-dioxolan-2-yl)methyl)-8-(1-(2,2-difluoroethyl)-1H-pyrazolo[3,4-b]pyrazin-6-yl)-2,8-diazaspiro[4.5]decan-3-one (700 mg, 1.65 mmol, 1.0 equiv.) and 4-bromo-2-(trifluoromethyl)pyridine (558 mg, 2.48 mmol, 1.5 equiv.) as the starting materials to give 1-((1,3-dioxolan-2-yl)methyl)-8-(1-(2,2-difluoroethyl)-1H-pyrazolo[3,4-b]pyrazin-6-yl)-2,8-diazaspiro[4.5]decan-3-one (600 mg, 64%) as a white solid. MS m/z: 568 [M+H]⁺.

Step 6: 2-(8-(1-(2,2-difluoroethyl)-1H-pyrazolo[3,4-b]pyrazin-6-yl)-3-oxo-2-(2-(trifluoromethyl)pyridin-4-yl)-2,8-diazaspiro[4.5]decan-1-yl)acetaldehyde: A solution of 1-((1,3-dioxolan-2-yl)methyl)-8-(1-(2,2-difluoroethyl)-1H-pyrazolo[3,4-b]pyrazin-6-yl)-2,8-diazaspiro[4.5]decan-3-one (600 mg, 1.06 mmol, 1.0 equiv.) and TsOH (182 mg, 1.06 mmol, 1.0 equiv) in acetone (10 mL)/H₂O (5 mL) was stirred for overnight at 60° C. The resulting mixture was concentrated under vacuum. The residue was purified by silica gel column chromatography, eluted with CH₂Cl₂/MeOH (10:1) to afford 2-(8-(1-(2,2-difluoroethyl)-1H-pyrazolo[3,4-b]pyrazin-6-yl)-3-oxo-2-(2-(trifluoromethyl)pyridin-4-yl)-2,8-diazaspiro[4.5]decan-1-yl)acetaldehyde (100 mg, 18%) as a yellow solid. MS m/z: 524 [M+H]⁺.

Step 7: 1-(2,2-difluoroethyl)-8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-3-one: Followed the general procedure AD using 2-(8-(1-(2,2-difluoroethyl)-1H-pyrazolo[3,4-b]pyrazin-6-yl)-3-oxo-2-(2-(trifluoromethyl)pyridin-4-yl)-2,8-diazaspiro[4.5]decan-1-yl)acetaldehyde (100 mg, 0.176 mmol, 1.0 equiv.) as the starting material to give 1-(2,2-difluoroethyl)-8-(1-(2,2-difluoroethyl)-1H-pyrazolo[3,4-b]pyrazin-6-yl)-2-(2-(trifluoromethyl)pyridin-4-yl)-2,8-diazaspiro[4.5]decan-3-one (20.8 mg, 22%) as a white solid. ¹H NMR (300 MHz, DMSO-d₆) δ 8.71 (d, J=5.6 Hz, 1H), 8.50 (s, 1H), 8.26 (d, J=2.1 Hz, 1H), 8.15 (s, 1H), 7.88 (dd, J=5.8, 2.1 Hz, 1H), 6.66-5.89 (m, 2H), 4.70 (td, J=15.0, 3.8 Hz, 2H), 4.58 (t, J=6.0 Hz, 1H), 4.34-4.21 (m, 2H), 3.58-3.42 (m, 1H), 3.36-3.33 (m, 1H), 2.91 (d, J=17.3 Hz, 1H), 2.74 (d, J=17.2 Hz, 1H), 2.47-2.35 (m, 1H), 2.32-2.11 (t, J=16.2 Hz, 1H), 1.89-1.72 (d, J=11.2 Hz, 3H), 1.71-1.61 (d, J=13.4 Hz, 1H). MS m/z: 546.1 [M+H]⁺.

### Example 956: 8-(1-(2,2-difluoroethyl)-1H-pyrazolo[3,4-b]pyrazin-6-yl)-2-(5-methyl-2-(trifluoromethyl)pyrimidin-4-yl)-2,8-diazaspiro[4.5]decan-3-one

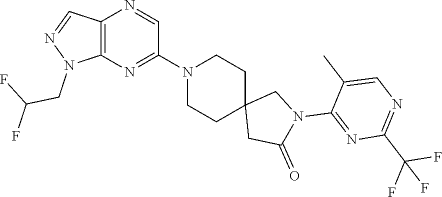 [SMILES: Cc1cnc(C(F)(F)F)nc1N1CC2(CCN(c3cnc4cnn(CC(F)F)c4n3)CC2)CC1=O]

Step 1: 2-(5-methyl-2-(trifluoromethyl)pyrimidin-4-yl)-2,8-diazaspiro[4.5]decan-3-one: Followed the general procedure H using tert-butyl 2-(5-methyl-2-(trifluoromethyl)pyrimidin-4-yl)-3-oxo-2,8-diazaspiro[4.5]decane-8-carboxylate (100 mg) as the starting material to give the crude product 2-(5-methyl-2-(trifluoromethyl)pyrimidin-4-yl)-2,8-diazaspiro[4.5]decan-3-one (80 mg). MS m/z: 315 [M+H]⁺.

Step 2: 8-(1-(2,2-difluoroethyl)-1H-pyrazolo[3,4-b]pyrazin-6-yl)-2-(5-methyl-2-(trifluoromethyl)pyrimidin-4-yl)-2,8-diazaspiro[4.5]decan-3-one: Followed the general procedure I using 2-[5-methyl-2-(trifluoromethyl)pyrimidin-4-yl]-2,8-diazaspiro[4.5]decan-3-one (80 mg, 0.255 mmol, 1.0 equiv.) and 6-chloro-1-(2,2-difluoroethyl)pyrazolo[3,4-b]pyrazine (66.76 mg, 0.306 mmol, 1.2 equiv.) as the starting materials to give 8-(1-(2,2-difluoroethyl)-1H-pyrazolo[3,4-b]pyrazin-6-yl)-2-(5-methyl-2-(trifluoromethyl)pyrimidin-4-yl)-2,8-diazaspiro[4.5]decan-3-one (91.5 mg, 72%) as a white solid. ¹H NMR (400 MHz, DMSO-d₆) δ 8.93 (s, 1H), 8.50 (s, 1H), 8.14 (s, 1H), 6.44 (tt, J=54.9, 3.8 Hz, 1H), 4.70 (td, J=15.0, 3.8 Hz, 2H), 3.96 (dt, J=13.9, 5.2 Hz, 2H), 3.91 (s, 2H), 3.71 (dt, J=13.2, 5.7 Hz, 2H), 2.66 (s, 2H), 2.29 (s, 3H), 1.80 (t, J=5.7 Hz, 4H). MS m/z: 497.15 [M+H]⁺.

### Example 957: 1-(2,2-difluoroethyl)-6-(2-(4-methyl-2-(trifluoromethyl)pyrimidin-5-yl)-2,6-diazaspiro[3.4]octan-6-yl)-1H-pyrazolo[3,4-b]pyrazine

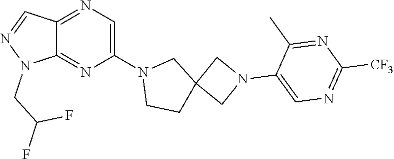 [SMILES: Cc1nc(C(F)(F)F)ncc1N1CC2(CCN(c3cnc4cnn(CC(F)F)c4n3)C2)C1]

Step 1: tert-butyl 2-(2-(trifluoromethyl)pyrimidin-5-yl)-2,6-diazaspiro[3.4]octane-6-carboxylate: Followed the general procedure Y using tert-butyl 2,6-diazaspiro[3.4]octane-6-carboxylate (200 mg, 0.942 mmol, 1.00 equiv.) and 5-bromo-2-(trifluoromethyl)pyrimidine (214 mg, 0.942 mmol, 1.0 equiv.) as the starting materials, Xphos (63.3 mg, 0.094 mmol, 0.100 equiv.) and Xphos Pd G3 (79.7 mg, 0.094 mmol, 0.100 equiv.) as the catalyst to give tert-butyl 2-(2-(trifluoromethyl)pyrimidin-5-yl)-2,6-diazaspiro[3.4]octane-6-carboxylate (250 mg, 74.2%) as a light yellow solid. MS m/z: 359 [M+H]⁺.

Step 2: tert-butyl 2-(4-bromo-2-(trifluoromethyl)pyrimidin-5-yl)-2,6-diazaspiro[3.4]octane-6-carboxylate: Followed the general procedure V using tert-butyl 2-(2-(trifluoromethyl)pyrimidin-5-yl)-2,6-diazaspiro[3.4]octane-6-carboxylate (250 mg, 0.572 mmol, 1.00 equiv.) as the starting material, NBS (102 mg, 0.572 μmol, 1.0 equiv.) as the reagent to give tert-butyl 2-(4-bromo-2-(trifluoromethyl)pyrimidin-5-yl)-2,6-diazaspiro[3.4]octane-6-carboxylate (110 mg, 36.1%) as a light yellow solid. MS m/z: 438 [M+H]⁺.

Step 3: tert-butyl 2-(4-methyl-2-(trifluoromethyl)pyrimidin-5-yl)-2,6-diazaspiro[3.4]octane-6-carboxylate: Followed the general procedure L using tert-butyl 2-(4-bromo-2-(trifluoromethyl)pyrimidin-5-yl)-2,6-diazaspiro[3.4]octane-6-carboxylate (110 mg, 0.251 mmol, 1.00 equiv.) and methylboronic acid (75.3 mg, 1.26 mmol, 5.00 equiv.) as the starting materials to give tert-butyl 2-(4-methyl-2-(trifluoromethyl)pyrimidin-5-yl)-2,6-diazaspiro[3.4]octane-6-carboxylate (80 mg, 85.4%) as a light yellow solid. MS m/z: 373 [M+H]⁺.

Step 4: 2-(4-methyl-2-(trifluoromethyl)pyrimidin-5-yl)-2,6-diazaspiro[3.4]octane: Followed the general procedure H using tert-butyl 2-(4-methyl-2-(trifluoromethyl)pyrimidin-5-yl)-2,6-diazaspiro[3.4]octane-6-carboxylate (80 mg, 0.214, 1.00 equiv.) as the starting material to give the crude product 2-(4-methyl-2-(trifluoromethyl)pyrimidin-5-yl)-2,6-diazaspiro[3.4]octane (65.0 mg). MS m/z: 273 [M+H]⁺.

Step 5: 1-(2,2-difluoroethyl)-6-(2-(4-methyl-2-(trifluoromethyl)pyrimidin-5-yl)-2,6-diazaspiro[3.4]octan-6-yl)-1H-pyrazolo[3,4-b]pyrazine: Followed the general procedure I using 2-(4-methyl-2-(trifluoromethyl)pyrimidin-5-yl)-2,6-diazaspiro[3.4]octane (65.0 mg, 0.239 mmol, 1.00 equiv.) and 6-chloro-1-(2,2-difluoroethyl)-1H-pyrazolo[3,4-b]pyrazine (52.2 mg, 0.239 mmol, 1.00 equiv.) as the starting materials to give 1-(2,2-difluoroethyl)-6-(2-(4-methyl-2-(trifluoromethyl)pyrimidin-5-yl)-2,6-diazaspiro[3.4]octan-6-yl)-1H-pyrazolo[3,4-b]pyrazine (20.0 mg, 17.1%) as a white solid. ¹H NMR (300 MHz, DMSO-d6) δ 8.13 (d, 2H), 7.98 (s, 1H), 6.67-6.26 (m, 1H), 4.79-4.59 (m, 2H), 4.24-4.11 (m, 4H), 3.86 (s, 2H), 3.68 (t, 2H), 2.46 (s, 3H), 2.34 (d, 2H). MS m/z: 455.1 [M+H]⁺.

### Example 958: 1-(2,2-difluoroethyl)-6-(2-(2-(difluoromethyl)pyrimidin-5-yl)-2,6-diazaspiro[3.4]octan-6-yl)-1H-pyrazolo[3,4-b]pyrazine

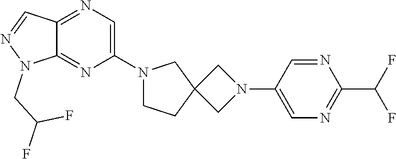 [SMILES: FC(F)Cn1ncc2ncc(N3CCC4(CN(c5cnc(C(F)F)nc5)C4)C3)nc21]

Step 1: 1-(2,2-difluoroethyl)-6-(2-(2-(difluoromethyl)pyrimidin-5-yl)-2,6-diazaspiro[3.4]octan-6-yl)-1H-pyrazolo[3,4-b]pyrazine: Followed the general procedure Y using 1-(2,2-difluoroethyl)-6-(2,6-diazaspiro[3.4]octan-6-yl)-1H-pyrazolo[3,4-b]pyrazine (50 mg, 170 μmol, 1.0 equiv.) and 5-bromo-2-(difluoromethyl)pyrimidine (71 mg, 340 μmol, 2.0 equiv.) as the starting materials to give 1-(2,2-difluoroethyl)-6-(2-(2-(difluoromethyl)pyrimidin-5-yl)-2,6-diazaspiro[3.4]octan-6-yl)-1H-pyrazolo[3,4-b]pyrazine (12.8 mg, 18%) as a white solid. ¹H NMR (300 MHz, DMSO-d₆) δ 8.22-8.06 (m, 4H), 7.06-6.19 (m, 2H), 4.79-4.60 (m, 2H), 4.15-3.99 (m, 4H), 3.87 (s, 2H), 3.67 (t, J=6.8 Hz, 2H), 2.33 (t, J=6.8 Hz, 2H). MS m/z: 423.15 [M+H]⁺.

### Example 959: 1-(2,2-difluoroethyl)-6-(2-(4-methyl-2-(2,2,2-trifluoroethoxy)pyrimidin-5-yl)-2,6-diazaspiro[3.4]octan-6-yl)-1H-pyrazolo[3,4-b]pyrazine

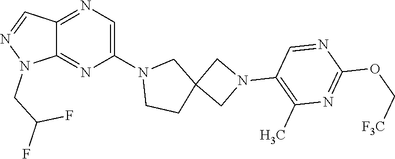 [SMILES: Cc1nc(OCC(F)(F)F)ncc1N1CC2(CCN(c3cnc4cnn(CC(F)F)c4n3)C2)C1]

Step 1: 5-bromo-4-methyl-2-(2,2,2-trifluoroethoxy)pyrimidine: Followed the general procedure E using 5-bromo-2-chloro-4-methylpyrimidine (250 mg, 1.21 mmol, 1.0 equiv.) and trifluoroethanol (145 mg, 1.45 mmol, 1.20 equiv.) and NaH (96.4 mg, 2.41 mmol, 2 equiv, 60%) as the starting materials to give 5-bromo-4-methyl-2-(2,2,2-trifluoroethoxy)pyrimidine (100 mg, 30%) as a white solid. MS m/z: 271 [M+H]⁺.

Step 2: tert-butyl 2-(4-methyl-2-(2,2,2-trifluoroethoxy)pyrimidin-5-yl)-2,6-diazaspiro[3.4]octane-6-carboxylate: Followed the general procedure Y using 5-bromo-4-methyl-2-(2,2,2-trifluoroethoxy)pyrimidine (100 mg, 0.369 mmol, 1.0 equiv.) and tert-butyl 2,6-diazaspiro[3.4]octane-6-carboxylate (94.0 mg, 0.443 mmol, 1.2 equiv.) as the starting materials, XPhos Pd G3 (62.5 mg, 0.0740 mmol, 0.2 equiv) as the catalyst to give tert-butyl 2-(4-methyl-2-(2,2,2-trifluoroethoxy)pyrimidin-5-yl)-2,6-diazaspiro[3.4]octane-6-carboxylate (100 mg, 62%) as a white solid. MS m/z: 403 [M+H]⁺.

Step 3: 2-(4-methyl-2-(2,2,2-trifluoroethoxy)pyrimidin-5-yl)-2,6-diazaspiro[3.4]octane: Followed the general procedure H using 2-[4-methyl-2-(2,2,2-trifluoroethoxy)pyrimidin-5-yl]-2,6-diazaspiro[3.4]octane-6-carboxylate (100 mg) as the starting material to give the crude product 2-(4-methyl-2-(2,2,2-trifluoroethoxy)pyrimidin-5-yl)-2,6-diazaspiro[3.4]octane (80 mg). MS m/z: 303 [M+H]⁺.

Step 4: 1-(2,2-difluoroethyl)-6-(2-(4-methyl-2-(2,2,2-trifluoroethoxy)pyrimidin-5-yl)-2,6-diazaspiro[3.4]octan-6-yl)-1H-pyrazolo[3,4-b]pyrazine: Followed the general procedure I using 2-(4-methyl-2-(2,2,2-trifluoroethoxy)pyrimidin-5-yl)-2,6-diazaspiro[3.4]octane (80 mg, 0.264 mmol, 1.0 equiv.) and 6-chloro-1-(2,2-difluoroethyl)-1H-pyrazolo[3,4-b]pyrazine (70 mg, 0.316 mmol, 1.2 equiv.) as the starting materials to give 1-(2,2-difluoroethyl)-6-(2-(4-methyl-2-(2,2,2-trifluoroethoxy)pyrimidin-5-yl)-2,6-diazaspiro[3.4]octan-6-yl)-1H-pyrazolo[3,4-b]pyrazine (44 mg, 34%) as a white solid. ¹H NMR (400 MHz, DMSO-d₆) δ 8.12 (d, J=10.5 Hz, 2H), 7.82 (s, 1H), 6.60-6.32 (m, 1H), 4.92 (q, J=9.1 Hz, 2H), 4.69 (td, J=14.9, 3.8 Hz, 2H), 3.94 (q, J=7.2 Hz, 4H), 3.84 (s, 2H), 3.67 (t, J=6.9 Hz, 2H), 2.33 (d, J=13.2 Hz, 5H). MS m/z: 485.10 [M+H]⁺.

### Example 960: 1-(2,2-difluoroethyl)-6-(2-(2-(2,2,2-trifluoroethoxy)pyrimidin-4-yl)-2,6-diazaspiro[3.4]octan-6-yl)-1H-pyrazolo[3,4-b]pyrazine

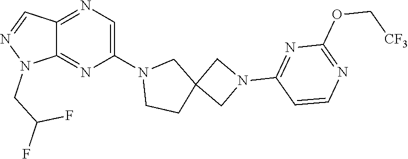 [SMILES: FC(F)Cn1ncc2ncc(N3CCC4(CN(c5ccnc(OCC(F)(F)F)n5)C4)C3)nc21]

Step 1: tert-butyl 6-(1-(2,2-difluoroethyl)-1H-pyrazolo[3,4-b]pyrazin-6-yl)-2,6-diazaspiro[3.4]octane-2-carboxylate: Followed the general procedure I using tert-butyl 2,6-diazaspiro[3.4]octane-2-carboxylate (225 mg, 1.06 mmol, 1.0 equiv) and 6-chloro-1-(2,2-difluoroethyl)-1H-pyrazolo[3,4-b]pyrazine (230 mg, 1.06 mmol, 1.0 equiv.) as the starting materials to give tert-butyl 6-(1-(2,2-difluoroethyl)-1H-pyrazolo[3,4-b]pyrazin-6-yl)-2,6-diazaspiro[3.4]octane-2-carboxylate (270 mg, 65%) as a white solid. MS m/z: 395 [M+H]⁺.

Step 2: 1-(2,2-difluoroethyl)-6-(2,6-diazaspiro[3.4]octan-6-yl)-1H-pyrazolo[3,4-b]pyrazine: Followed the general procedure H using tert-butyl 6-(1-(2,2-difluoroethyl)-1H-pyrazolo[3,4-b]pyrazin-6-yl)-2,6-diazaspiro[3.4]octane-2-carboxylate (270 mg) as the starting material to give the crude product 1-(2,2-difluoroethyl)-6-(2,6-diazaspiro[3.4]octan-6-yl)-1H-pyrazolo[3,4-b]pyrazine (350 mg). MS m/z: 295 [M+H]⁺.

Step 3: 6-(2-(2-chloropyrimidin-4-yl)-2,6-diazaspiro[3.4]octan-6-yl)-1-(2,2-difluoroethyl)-1H-pyrazolo[3,4-b]pyrazine: Followed the general procedure Y using 1-(2,2-difluoroethyl)-6-(2,6-diazaspiro[3.4]octan-6-yl)-1H-pyrazolo[3,4-b]pyrazine (350 mg, 0.68 mmol, 1.0 equiv.) and 4-bromo-2-chloropyrimidine (200 mg, 1.02 mmol, 1.5 equiv.) as the starting materials to give 6-(2-(2-chloropyrimidin-4-yl)-2,6-diazaspiro[3.4]octan-6-yl)-1-(2,2-difluoroethyl)-1H-pyrazolo[3,4-b]pyrazine (110 mg, 40%) as a white solid. MS m/z: 407 [M+H]⁺.

Step 4: 1-(2,2-difluoroethyl)-6-(2-(2-(2,2,2-trifluoroethoxy)pyrimidin-4-yl)-2,6-diazaspiro[3.4]octan-6-yl)-1H-pyrazolo[3,4-b]pyrazine: Followed the general procedure E using 6-(2-(2-chloropyrimidin-4-yl)-2,6-diazaspiro[3.4]octan-6-yl)-1-(2,2-difluoroethyl)-1H-pyrazolo[3,4-b]pyrazine (55 mg, 0.14 mmol, 1.0 equiv.) and CF₂CH₂OH (20 mg, 0.2 mmol, 1.5 equiv.) as the starting materials to give 1-(2,2-difluoroethyl)-6-(2-(2-(2,2,2-trifluoroethoxy)pyrimidin-4-yl)-2,6-diazaspiro[3.4]octan-6-yl)-1H-pyrazolo[3,4-b]pyrazine (30 mg, 47%) as a white solid. ¹H NMR (400 MHz, DMSO-d₆) δ 8.14 (s, 1H), 8.10 (s, 1H), 8.03 (d, J=5.9 Hz, 1H), 6.46 (tt, J=54.9, 3.9 Hz, 1H), 6.18 (d, J=5.8 Hz, 1H), 4.91 (q, J=9.1 Hz, 2H), 4.68 (td, J=14.9, 3.9 Hz, 2H), 4.22-4.01 (m, 4H), 3.86 (s, 2H), 3.67 (t, J=6.8 Hz, 2H), 2.37-2.24 (m, 2H). MS m/z: 471.1 [M+H]⁺.

### Example 961: 1-(2,2-difluoroethyl)-6-(2-(5-methyl-2-(trifluoromethyl)pyrimidin-4-yl)-2,6-diazaspiro[3.4]octan-6-yl)-1H-pyrazolo[3,4-b]pyrazine

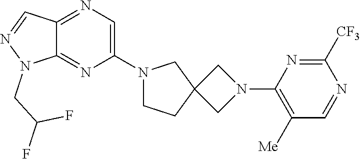 [SMILES: Cc1cnc(C(F)(F)F)nc1N1CC2(CCN(c3cnc4cnn(CC(F)F)c4n3)C2)C1]

Step 1: tert-butyl 2-(5-bromo-2-(trifluoromethyl)pyrimidin-4-yl)-2,6-diazaspiro[3.4]octane-6-carboxylate: Followed the general procedure W using tert-butyl 2-(2-(trifluoromethyl)pyrimidin-4-yl)-2,6-diazaspiro[3.4]octane-6-carboxylate (200 mg, 0.558 mmol, 1 equiv) as the starting material, NBS (298.00 mg, 1.674 mmol, 3 equiv.) as the reagent to give tert-butyl 2-(5-bromo-2-(trifluoromethyl)pyrimidin-4-yl)-2,6-diazaspiro[3.4]octane-6-carboxylate (150 mg, 71%) as a white solid MS m/z: 437 [M+H]⁺.

Step 2: tert-butyl 2-(5-methyl-2-(trifluoromethyl)pyrimidin-4-yl)-2,6-diazaspiro[3.4]octane-6-carboxylate: Followed the general procedure L using tert-butyl 2-[5-bromo-2-(trifluoromethyl)pyrimidin-4-yl]-2,6-diazaspiro[3.4]octane-6-carboxylate (150 mg, 0.343 mmol, 1 equiv) and methylboronic acid (25 mg, 0.412 mmol, 1.2 equiv.) as the starting materials, Pd(dppf)Cl₂ (22 mg, 0.034 mmol, 0.1) equiv. as the catalyst to give tert-butyl 2-(5-methyl-2-(trifluoromethyl)pyrimidin-4-yl)-2,6-diazaspiro[3.4]octane-6-carboxylate (100 mg, 78%) as a white solid MS m/z: 373 [M+H]⁺.

Step 3: 2-(5-methyl-2-(trifluoromethyl)pyrimidin-4-yl)-2,6-diazaspiro[3.4]octane: Followed the general procedure H using tert-butyl 2-(5-methyl-2-(trifluoromethyl)pyrimidin-4-yl)-2,6-diazaspiro[3.4]octane-6-carboxylate (100 mg, 0.269 mmol, 1 equiv) as the starting material to give the crude product 2-(5-methyl-2-(trifluoromethyl)pyrimidin-4-yl)-2,6-diazaspiro[3.4]octane (70 mg). MS m/z: 273 [M+H]⁺.

Step 4: 1-(2,2-difluoroethyl)-6-(2-(5-methyl-2-(trifluoromethyl)pyrimidin-4-yl)-2,6-diazaspiro[3.4]octan-6-yl)-1H-pyrazolo[3,4-b]pyrazine: Followed the general procedure I using 2-(5-methyl-2-(trifluoromethyl)pyrimidin-4-yl)-2,6-diazaspiro[3.4]octane (70 mg, 0.257 mmol, 1 equiv) and 6-chloro-1-(2,2-difluoroethyl)-1H-pyrazolo[3,4-b]pyrazine (67.4 mg, 0.308 mmol, 1.2 equiv) as the starting materials to give 1-(2,2-difluoroethyl)-6-(2-(5-methyl-2-(trifluoromethyl)pyrimidin-4-yl)-2,6-diazaspiro[3.4]octan-6-yl)-1H-pyrazolo[3,4-b]pyrazine (40 mg, 34%) as a white solid. ¹H NMR (400 MHz, DMSO-d₆) δ 8.14 (s, 1H), 8.10-8.07 (m, 2H), 6.46 (tt, J=55.0, 3.9 Hz, 1H), 4.68 (td, J=14.9, 3.9 Hz, 2H), 4.58-4.12 (m, 4H), 3.86 (s, 2H), 3.67 (t, J=6.9 Hz, 2H), 2.30 (t, J=6.8 Hz, 2H), 2.23 (s, 3H). MS m/z: 455.10 [M+H]⁺.

### Example 962: 1-(oxetan-3-yl)-6-(2-(2-(trifluoromethyl)pyridin-4-yl)-2,6-diazaspiro[3.4]octan-6-yl)-1H-pyrazolo[3,4-d]pyrimidine

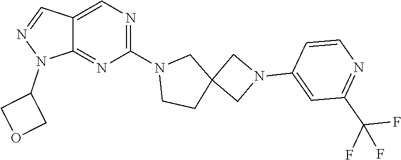 [SMILES: FC(F)(F)c1cc(N2CC3(CCN(c4ncc5cnn(C6COC6)c5n4)C3)C2)ccn1]

Step 1: 6-chloro-1-(2,2-difluoroethyl)-3-methyl-1H-pyrazolo[3,4-d]pyrimidine: Followed the general procedure K using 6-chloro-3-methyl-1H-pyrazolo[3,4-d]pyrimidine (150 mg, 0.86 mmol, 1.0 equiv.) and 2,2-difluoroethyl trifluoromethanesulfonate (211 mg, 1.02 mmol, 1.5 equiv.) as the starting materials to give 6-chloro-1-(2,2-difluoroethyl)-3-methyl-1H-pyrazolo[3,4-d]pyrimidine (70 mg, 49%) as a white solid. MS m/z: 211 [M+H]⁺.

Step 2: 1-(oxetan-3-yl)-6-(2-(2-(trifluoromethyl)pyridin-4-yl)-2,6-diazaspiro[3.4]octan-6-yl)-1H-pyrazolo[3,4-d]pyrimidine: Followed the general procedure I using 6-chloro-1-(2,2-difluoroethyl)-3-methyl-1H-pyrazolo[3,4-d]pyrimidine (70 mg, 0.322 mmol, 1.0 equiv.) and 2-(2-(trifluoromethyl)pyridin-4-yl)-2,6-diazaspiro[3.4]octane (103 mg, 0.398 mmol, 1.2 equiv.) as the starting materials to give 1-(oxetan-3-yl)-6-(2-(2-(trifluoromethyl)pyridin-4-yl)-2,6-diazaspiro[3.4]octan-6-yl)-1H-pyrazolo[3,4-d]pyrimidine (32.5 mg, 23%) as a white solid. ¹H NMR (400 MHz, DMSO-d₆) δ 8.91 (s, 1H), 8.25 (d, J=5.7 Hz, 1H), 8.15 (d, J=7.0 Hz, 1H), 6.75 (d, J=2.3 Hz, 1H), 6.57 (dd, J=5.7, 2.2 Hz, 1H), 5.87 (p, J=7.2 Hz, 1H), 5.04 (t, J=6.4 Hz, 2H), 4.96 (t, J=7.1 Hz, 2H), 4.11-3.94 (m, 4H), 3.83 (s, 2H), 3.66 (s, 2H), 2.27 (t, J=6.9 Hz, 2H). MS m/z: 432.15 [M+H]⁺.

### Example 963: 8-(1-(2,2-difluoroethyl)-1H-pyrazolo[3,4-b]pyrazin-6-yl)-2-((3-fluoro-5-(trifluoromethyl)pyridin-2-yl)methyl)-2,8-diazaspiro[4.5]decan-3-one

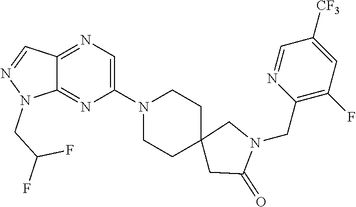 [SMILES: O=C1CC2(CCN(c3cnc4cnn(CC(F)F)c4n3)CC2)CN1Cc1ncc(C(F)(F)F)cc1F]

Step 1: (3-fluoro-5-(trifluoromethyl)pyridin-2-yl)methyl methanesulfonate: A mixture of 2-bromo-3-fluoro-5-(trifluoromethyl)pyridine (600 mg, 2.45 mmol, 1.00 equiv.), Pd(PPh₃)₄ (284 mg, 0.246 mmol, 0.100 equiv.) and (tributylstannyl)methanol (1579 mg, 4.91 mmol, 2.00 equiv.) in Toluene (6 mL) was stirred for 16 h at 80° C. under argon atmosphere. To the above mixture was added TEA (497 mg, 4.91 mmol, 2.00 equiv) and methanesulfonic anhydride (514 mg, 2.95 mmol, 1.20 equiv.) in DCM (6 mL) at room temperature. The resulting mixture was stirred for additional 2 h at room temperature. The resulting mixture was extracted with EtOAc (3×20 mL). The combined organic layers were washed with water (2×20 mL), dried over anhydrous Na₂SO₄. After filtration, the filtrate was concentrated under reduced pressure. The residue was purified by silica gel column chromatography, eluted with EtOAc/PE=1/1 to afford (3-fluoro-5-(trifluoromethyl)pyridin-2-yl)methyl methanesulfonate (60 mg, 8.93%) as a white solid. MS m/z: 274 [M+H]⁺.

Step 2: 8-(1-(2,2-difluoroethyl)-1H-pyrazolo[3,4-b]pyrazin-6-yl)-2-((3-fluoro-5-(trifluoromethyl)pyridin-2-yl)methyl)-2,8-diazaspiro[4.5]decan-3-one: Followed the general procedure E using 8-(1-(2,2-difluoroethyl)-1H-pyrazolo[3,4-b]pyrazin-6-yl)-2,8-diazaspiro[4.5]decan-3-one hydrochloride (75.0 mg, 0.201 mmol, 1.10 equiv.) and (3-fluoro-5-(trifluoromethyl)pyridin-2-yl)methyl methanesulfonate (50.0 mg, 0.183 mmol, 1.00 equiv.) as the starting materials to give 8-(1-(2,2-difluoroethyl)-1H-pyrazolo[3,4-b]pyrazin-6-yl)-2-((3-fluoro-5-(trifluoromethyl)pyridin-2-yl)methyl)-2,8-diazaspiro[4.5]decan-3-one (26.2 mg, 26.1%) as a white solid. ¹H NMR (400 MHz, DMSO-d₆) δ 8.83 (s, 1H), 8.47 (s, 1H), 8.37-8.29 (m, 1H), 8.12 (s, 1H), 6.60-6.27 (m, 1H), 4.76-4.63 (m, 4H), 3.88-3.79 (m, 2H), 3.75-3.64 (m, 2H), 2.34 (s, 2H), 1.67 (t, 4H). MS m/z: 518.2 [M+H]⁺.

### Example 964: 8-(1-(2,2-difluoroethyl)-1H-pyrazolo[3,4-b]pyrazin-6-yl)-2-(1-(5-(trifluoromethyl)pyridin-2-yl)ethyl)-2,8-diazaspiro[4.5]decan-3-one

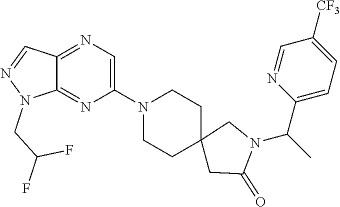 [SMILES: CC(c1ccc(C(F)(F)F)cn1)N1CC2(CCN(c3cnc4cnn(CC(F)F)c4n3)CC2)CC1=O]

Step 1: 1-(5-(trifluoromethyl)pyridin-2-yl)ethan-1-ol: Followed the general procedure O using 1-(5-(trifluoromethyl)pyridin-2-yl)ethan-1-one (500 mg, 2.644 mmol, 1.0 equiv.) as the starting material to give 1-(5-(trifluoromethyl)pyridin-2-yl)ethan-1-ol (320 mg, 44%) as a colorless oil. MS m/z: 192 [M+H]⁺.

Step 2: 1-(5-(trifluoromethyl)pyridin-2-yl)ethyl methanesulfonate: Followed the general procedure R using 1-(5-(trifluoromethyl)pyridin-2-yl)ethanol (300 mg, 3.13 mmol, 1 equiv.) and methanesulfonic anhydride (410 mg, 4.70 mmol, 1.5 equiv.) as the starting materials to give 1-(5-(trifluoromethyl)pyridin-2-yl)ethyl methanesulfonate (120 mg, 39%) as a light yellow oil. MS m/z: 270 [M+H]⁺.

Step 3: 8-(1-(2,2-difluoroethyl)-1H-pyrazolo[3,4-b]pyrazin-6-yl)-2-(1-(5-(trifluoromethyl)pyridin-2-yl)ethyl)-2,8-diazaspiro[4.5]decan-3-one: Followed the general procedure Y using 1-(5-(trifluoromethyl)pyridin-2-yl)ethyl methanesulfonate (100 mg, 0.371 mmol, 1.0 equiv.) and 8-(1-(2,2-difluoroethyl)-1H-pyrazolo[3,4-b]pyrazin-6-yl)-2,8-diazaspiro[4.5]decan-3-one (124 mg, 0.371 mmol, 1.0 equiv.) as the starting materials, xPhos (35.4 mg, 0.074 mmol, 0.2 equiv.), xPhos Pd G3 (31.4 mg, 0.037 mmol, 0.1 equiv.) as the catalysts to give 8-(1-(2,2-difluoroethyl)-1H-pyrazolo[3,4-b]pyrazin-6-yl)-2-(1-(5-(trifluoromethyl)pyridin-2-yl)ethyl)-2,8-diazaspiro[4.5]decan-3-one (7.7 mg, 3.9%) as a white solid. ¹H NMR (400 MHz, Chloroform-d) δ 8.83 (s, 2H), 8.13-7.84 (m, 2H), 7.61-7.32 (m, 1H), 6.40-6.08 (m, 1H), 5.54 (d, J=7.2 Hz, 1H), 4.81-4.49 (m, 2H), 4.00-3.77 (m, 2H), 3.74-3.55 (m, 2H), 3.41-3.19 (m, 2H), 2.54-2.25 (m, 2H), 1.89-1.73 (m, 2H), 1.68-1.63 (m, 3H), 1.21-1.08 (m, 1H), 1.08-0.91 (m, 1H). MS m/z: 510.1 [M+H]⁺.

### Example 965: 8-(1-(2,2-difluoroethyl)-1H-pyrazolo[3,4-b]pyrazin-6-yl)-2-(1-(6-(trifluoromethyl)pyridin-3-yl)ethyl)-2,8-diazaspiro[4.5]decan-3-one

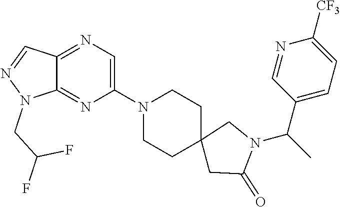 [SMILES: CC(c1ccc(C(F)(F)F)nc1)N1CC2(CCN(c3cnc4cnn(CC(F)F)c4n3)CC2)CC1=O]

Step 1: 1-(6-(trifluoromethyl)pyridin-3-yl)ethyl methanesulfonate: Followed the general procedure R using 1-(6-(trifluoromethyl)pyridin-3-yl)ethan-1-ol (90 mg, 471 μmol, 1.0 equiv.) and MsCl (64.7 mg, 565 μmol, 1.2 equiv.) as the starting materials to give 1-(6-(trifluoromethyl)pyridin-3-yl)ethyl methanesulfonate (115 mg, 91%) as a white solid. MS m/z: 270 [M+H]⁺.

Step 2: tert-butyl 3-oxo-2-(1-(6-(trifluoromethyl)pyridin-3-yl)ethyl)-2,8-diazaspiro[4.5]decane-8-carboxylate: Followed the general procedure E using tert-butyl 3-oxo-2,8-diazaspiro[4.5]decane-8-carboxylate (100 mg, 371 μmol, 1.0 equiv.) and 1-(6-(trifluoromethyl)pyridin-3-yl)ethyl methanesulfonate (115 mg, 371 μmol, 1.0 equiv.) as the starting materials to give tert-butyl 3-oxo-2-(1-(6-(trifluoromethyl)pyridin-3-yl)ethyl)-2,8-diazaspiro[4.5]decane-8-carboxylate (125 mg, 79%) as a white solid. MS m/z: [M+H]⁺.

Step 3: 2-(1-(6-(trifluoromethyl)pyridin-3-yl)ethyl)-2,8-diazaspiro[4.5]decan-3-one: Followed the general procedure B using tert-butyl 3-oxo-2-(1-(6-(trifluoromethyl)pyridin-3-yl)ethyl)-2,8-diazaspiro[4.5]decane-8-carboxylate (125 mg) as the starting material to give the crude product 2-(1-(6-(trifluoromethyl)pyridin-3-yl)ethyl)-2,8-diazaspiro[4.5]decan-3-one hydrochloride (100 mg). MS m/z: 328 [M+H]⁺.

Step 4: 8-(1-(2,2-difluoroethyl)-1H-pyrazolo[3,4-b]pyrazin-6-yl)-2-(1-(6-(trifluoromethyl)pyridin-3-yl)ethyl)-2,8-diazaspiro[4.5]decan-3-one: Followed the general procedure I using 2-(1-(6-(trifluoromethyl)pyridin-3-yl)ethyl)-2,8-diazaspiro[4.5]decan-3-one hydrochloride (100 mg, 299 μmol, 1.0 equiv.) and 6-chloro-1-(2,2-difluoroethyl)-1H-pyrazolo[3,4-b]pyrazine (78.5 mg, 359 μmol, 1.2 equiv.) as the starting materials to give 8-(1-(2,2-difluoroethyl)-1H-pyrazolo[3,4-b]pyrazin-6-yl)-2-(1-(6-(trifluoromethyl)pyridin-3-yl)ethyl)-2,8-diazaspiro[4.5]decan-3-one (78 mg, 50%) as a white solid. ¹H NMR (300 MHz, DMSO-d₆) δ 8.72 (d, J=2.2 Hz, 1H), 8.47 (s, 1H), 8.13 (s, 1H), 8.02 (dd, J=8.2, 2.2 Hz, 1H), 7.92 (d, J=8.2 Hz, 1H), 6.67-6.20 (m, 1H), 5.37 (q, J=7.1 Hz, 1H), 4.69 (td, J=15.1, 3.8 Hz, 2H), 4.01-3.78 (m, 2H), 3.73-3.55 (m, 2H), 3.32 (s, 1H), 3.00 (d, J=9.9 Hz, 1H), 2.36 (d, J=2.0 Hz, 2H), 1.66 (t, J=5.7 Hz, 2H), 1.55 (d, J=7.2 Hz, 5H). MS m/z: 510.2 [M+H]⁺.

### Example 966: 8-(1-(2,2-difluoroethyl)-1H-pyrazolo[3,4-b]pyrazin-6-yl)-2-((3-fluoro-2-(trifluoromethyl)pyridin-4-yl)methyl)-2,8-diazaspiro[4.5]decan-3-one

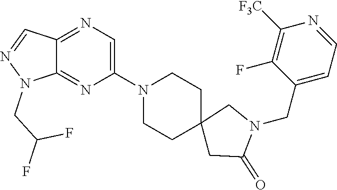 [SMILES: O=C1CC2(CCN(c3cnc4cnn(CC(F)F)c4n3)CC2)CN1Cc1ccnc(C(F)(F)F)c1F]

Step 1: (3-fluoro-2-(trifluoromethyl)pyridin-4-yl)methanol: Followed the general procedure AH using 3-fluoro-2-(trifluoromethyl)isonicotinic acid (150 mg, 0.718 mmol, 1.0 equiv.) as the starting material, BH₃-DMS (10 M, 0.2 mL, 4.0 equiv.) as the reductant to give (3-fluoro-2-(trifluoromethyl)pyridin-4-yl)methanol (90 mg, 60%) as a colorless oil. MS m/z: 196 [M+H]⁺.

Step 2: (3-fluoro-2-(trifluoromethyl)pyridin-4-yl)methyl methanesulfonate: Followed the general procedure R using (3-fluoro-2-(trifluoromethyl)pyridin-4-yl)methanol (90 mg, 0.459 mmol, 1.0 equiv.) and Ms₂O (161 mg, 0.920 mmol, 2.0 equiv.) as the starting materials to give (3-fluoro-2-(trifluoromethyl)pyridin-4-yl)methyl methanesulfonate (70.0 mg, 60%) as a white solid. MS m/z: 274 [M+H]⁺.

Step 3: 8-(1-(2,2-difluoroethyl)-1H-pyrazolo[3,4-b]pyrazin-6-yl)-2-((3-fluoro-2-(trifluoromethyl)pyridin-4-yl)methyl)-2,8-diazaspiro[4.5]decan-3-one: Followed the general procedure E using (3-fluoro-2-(trifluoromethyl)pyridin-4-yl)methyl methanesulfonate (70 mg, 0.255 mmol, 1.0 equiv.) and 8-(1-(2,2-difluoroethyl)-1H-pyrazolo[3,4-b]pyrazin-6-yl)-2,8-diazaspiro[4.5]decan-3-one (86 mg, 0.255 mmol, 1.0 equiv.) as the starting materials to give 8-(1-(2,2-difluoroethyl)-1H-pyrazolo[3,4-b]pyrazin-6-yl)-2-((3-fluoro-2-(trifluoromethyl)pyridin-4-yl)methyl)-2,8-diazaspiro[4.5]decan-3-one (42.6 mg, 60%) as a white solid. ¹H NMR (400 MHz, DMSO-d₆) δ 8.57 (d, 1H), 8.47 (s, 1H), 8.12 (s, 1H), 7.69 (t, 1H), 6.45 (tt, 1H), 4.71 (td, 2H), 4.60 (s, 2H), 3.93-3.79 (m, 2H), 3.75-3.57 (m, 2H), 3.25 (s, 2H), 2.40 (s, 2H), 1.67 (q, 4H). MS m/z: 514.15 [M+H]⁺.

### Example 967: 2-(1-(5-chloropyridin-2-yl)ethyl)-8-(1-(2,2-difluoroethyl)-1H-pyrazolo[3,4-b]pyrazin-6-yl)-2,8-diazaspiro[4.5]decan-3-one

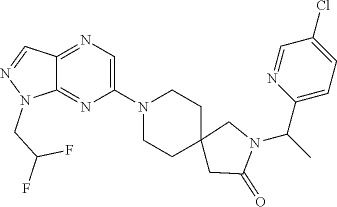 [SMILES: CC(c1ccc(Cl)cn1)N1CC2(CCN(c3cnc4cnn(CC(F)F)c4n3)CC2)CC1=O]

Step 1: 2-(1-bromoethyl)-5-chloropyridine: Followed the general procedure W using 5-chloro-2-ethylpyridine (150 mg, 1.06 mmol, 1.0 equiv.) as the starting material, NBS (283 mg, 1.59 mmol, 1.5 equiv.) as the reagent to give 2-(1-bromoethyl)-5-chloropyridine (150 mg, 64%) as a white solid. MS m/z: 220 [M+H]⁺.

Step 2: tert-butyl 2-(1-(5-chloropyridin-2-yl)ethyl)-3-oxo-2,8-diazaspiro[4.5]decane-8-carboxylate: Followed the general procedure E using 2-(1-bromoethyl)-5-chloropyridine (110 mg, 507 μmol, 1.5 equiv.) and tert-butyl 3-oxo-2,8-diazaspiro[4.5]decane-8-carboxylate (86 mg, 338 μmol, 1.0 equiv.) as the starting materials to give tert-butyl 2-(1-(5-chloropyridin-2-yl)ethyl)-3-oxo-2,8-diazaspiro[4.5]decane-8-carboxylate (80 mg, 60%) as a white solid. MS m/z: 394 [M+H]⁺.

Step 3: 2-(1-(5-chloropyridin-2-yl)ethyl)-2,8-diazaspiro[4.5]decan-3-one hydrochloride: Followed the general procedure B using tert-butyl 2-(1-(5-chloropyridin-2-yl)ethyl)-3-oxo-2,8-diazaspiro[4.5]decane-8-carboxylate (80 mg) as the starting material to give the crude product 2-(1-(5-chloropyridin-2-yl)ethyl)-2,8-diazaspiro[4.5]decan-3-one hydrochloride (55 mg). MS m/z: 294 [M+H]⁺.

Step 4: 2-(1-(5-chloropyridin-2-yl)ethyl)-8-(1-(2,2-difluoroethyl)-1H-pyrazolo[3,4-b]pyrazin-6-yl)-2,8-diazaspiro[4.5]decan-3-one: Followed the general procedure I using 2-(1-(5-chloropyridin-2-yl)ethyl)-2,8-diazaspiro[4.5]decan-3-one hydrochloride (55 mg, 187 μmol, 1.0 equiv.) and 6-chloro-1-(2,2-difluoroethyl)-1H-pyrazolo[3,4-b]pyrazine (49.1 mg, 224 μmol, 1.2 equiv.) as the starting materials to give 2-(1-(5-chloropyridin-2-yl)ethyl)-8-(1-(2,2-difluoroethyl)-1H-pyrazolo[3,4-b]pyrazin-6-yl)-2,8-diazaspiro[4.5]decan-3-one (43.7 mg, 48%) as a white solid. ¹H NMR (300 MHz, DMSO-d₆) δ 8.60 (d, J=2.5 Hz, 1H), 8.46 (s, 1H), 8.13 (s, 1H), 7.93 (dd, J=8.4, 2.6 Hz, 1H), 7.37 (d, J=8.5 Hz, 1H), 6.64-6.19 (m, 1H), 5.38-5.22 (m, 1H), 4.79-4.59 (m, 2H), 3.98-3.74 (m, 2H), 3.72-3.56 (m, 2H), 3.32-3.26 (m, 1H), 3.07 (d, J=9.8 Hz, 1H), 2.33 (d, J=2.1 Hz, 2H), 1.66 (t, J=5.6 Hz, 2H), 1.56 (s, 2H), 1.48 (d, J=7.1 Hz, 3H). MS m/z: 476.15 [M+H]⁺.

### Example 968: 2-(1-(5-chloropyridin-3-yl)ethyl)-8-(1-(2,2-difluoroethyl)-1H-pyrazolo[3,4-b]pyrazin-6-yl)-2,8-diazaspiro[4.5]decan-3-one

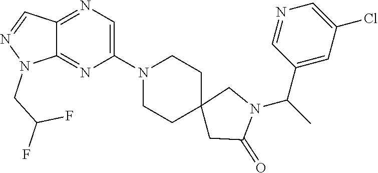 [SMILES: CC(c1cncc(Cl)c1)N1CC2(CCN(c3cnc4cnn(CC(F)F)c4n3)CC2)CC1=O]

Step 1: 1-(5-chloropyridin-3-yl)ethan-1-ol: Followed the general procedure P using 5-chloronicotinaldehyde (500 mg, 3.52 mmol, 1.0 equiv.) as the starting material to give 1-(5-chloropyridin-3-yl)ethan-1-ol (390 mg, 70%) as a white solid. MS m/z: 142 [M+H]⁺.

Step 2: 1-(5-chloropyridin-3-yl)ethyl methanesulfonate: Followed the general procedure R using 1-(5-chloropyridin-3-yl)ethan-1-ol (390 mg, 2.47 mmol, 1.0 equiv.) and methanesulfonic anhydride (519 mg, 2.96 mmol, 1.2 equiv.) as the starting materials to give 1-(5-chloropyridin-3-yl)ethyl methanesulfonate (400 mg, 68%) as a white solid. MS m/z: 158 [M+H]⁺.

Step 3: 2-(1-(5-chloropyridin-3-yl)ethyl)-8-(1-(2,2-difluoroethyl)-1H-pyrazolo[3,4-b]pyrazin-6-yl)-2,8-diazaspiro[4.5]decan-3-one: Followed the general procedure E using 8-(1-(2,2-difluoroethyl)-1H-pyrazolo[3,4-b]pyrazin-6-yl)-2,8-diazaspiro[4.5]decan-3-one (172 mg, 0.509 mmol, 1.2 equiv.) and 1-(5-chloropyridin-3-yl)ethyl methanesulfonate (100 mg, 0.424 mmol, 1.0 equiv.) as the starting materials to give 8-(1-(2,2-difluoroethyl)-1H-pyrazolo[3,4-b]pyrazin-6-yl)-2-(1-(2-(trifluoromethyl)pyrimidin-5-yl)ethyl)-2,8-diazaspiro[4.5]decan-3-one (10.6 mg, 5.6%) as an off-white solid. ¹H NMR (400 MHz, DMSO-d₆) δ 8.57 (d, J=2.3 Hz, 1H), 8.51-8.44 (m, 2H), 8.12 (s, 1H), 7.89-7.84 (m, 1H), 6.43 (tt, J=54.9, 3.8 Hz, 1H), 5.35-5.25 (m, 1H), 4.68 (td, J=15.1, 3.8 Hz, 2H), 3.94-3.76 (m, 2H), 3.71-3.57 (m, 2H), 3.3 (s, 1H), 2.99 (d, J=9.9 Hz, 1H), 2.34 (d, J=5.6 Hz, 2H), 1.65 (t, J=5.7 Hz, 2H), 1.58-1.45 (m, 5H). MS m/z: 476.1 [M+H]⁺.

### Example 969: 8-(1-(2,2-difluoroethyl)-1H-pyrazolo[3,4-b]pyrazin-6-yl)-2-(1-(2-(trifluoromethyl)pyridin-4-yl)ethyl)-2,8-diazaspiro[4.5]decan-3-one

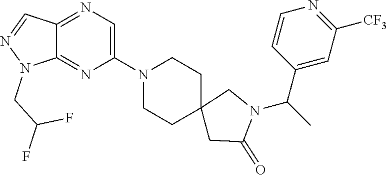 [SMILES: CC(c1ccnc(C(F)(F)F)c1)N1CC2(CCN(c3cnc4cnn(CC(F)F)c4n3)CC2)CC1=O]

Step 1: N-methoxy-N-methyl-2-(trifluoromethyl)isonicotinamide: Followed the general procedure G using 2-(trifluoromethyl)isonicotinic acid (80 mg, 0.325 mmol, 1 equiv) and N,O-dimethylhydroxylamine (500 mg, 0.488 mmol, 1.5 equiv) as the starting materials to give N-methoxy-N-methyl-2-(trifluoromethyl)isonicotinamide (500 mg, 81%) as a white liquid. MS m/z: 235 [M+H]⁺.

Step 2: 1-(2-(trifluoromethyl)pyridin-4-yl)ethan-1-one: Followed the general procedure P using N-methoxy-N-methyl-2-(trifluoromethyl)isonicotinamide (500 mg, 2.13 mmol, 1 equiv) as the starting material to give 1-(2-(trifluoromethyl)pyridin-4-yl)ethan-1-one (400 mg, 99%) as a white liquid. MS m/z: 190 [M+H]⁺.

Step 3: 1-(2-(trifluoromethyl)pyridin-4-yl)ethan-1-ol: Followed the general procedure Q using 1-(2-(trifluoromethyl)pyridin-4-yl)ethan-1-one (400 mg, 2.12 mmol, 1 equiv) and NaBH₄ (400 mg, 10.6 mmol, 5 equiv) as the starting material to give 1-(2-(trifluoromethyl)pyridin-4-yl)ethan-1-ol (320 mg, 79%) as a white liquid MS m/z: 192 [M+H]⁺.

Step 4: 1-(2-(trifluoromethyl)pyridin-4-yl)ethyl methanesulfonate: Followed the general procedure R using 1-[2-(trifluoromethyl)pyridin-4-yl]ethanol (320 mg, 1.67 mmol, 1 equiv) and Ms₂O (350 mg, 2.009 mmol, 1.2 equiv.) as the starting materials to give 1-(2-(trifluoromethyl)pyridin-4-yl)ethyl methanesulfonate (200 mg, 44%) as a white liquid. MS m/z: 270 [M+H]⁺.

Step 5: 8-(1-(2,2-difluoroethyl)-1H-pyrazolo[3,4-b]pyrazin-6-yl)-2-(1-(2-(trifluoromethyl)pyridin-4-yl)ethyl)-2,8-diazaspiro[4.5]decan-3-one: Followed the general procedure E using 8-(1-(2,2-difluoroethyl)-1H-pyrazolo[3,4-b]pyrazin-6-yl)-2,8-diazaspiro[4.5]decan-3-one (300 mg, 0.892 mmol, 1.2 equiv) and 1-(2-(trifluoromethyl)pyridin-4-yl)ethyl methanesulfonate (200 mg, 0.743 mmol, 1 equiv) as the starting materials to give 8-(1-(2,2-difluoroethyl)-1H-pyrazolo[3,4-b]pyrazin-6-yl)-2-(1-(2-(trifluoromethyl)pyridin-4-yl)ethyl)-2,8-diazaspiro[4.5]decan-3-one (100 mg, 25.6%) as a white solid. ¹H NMR (400 MHz, DMSO-d₆) δ 8.76 (d, J=5.0 Hz, 1H), 8.47 (s, 1H), 8.12 (s, 1H), 7.74 (s, 1H), 7.63 (d, 1H), 6.43 (tt, J=54.8, 3.8 Hz, 1H), 5.36-5.26 (m, 1H), 4.68 (td, J=15.0, 3.8 Hz, 2H), 3.95-3.78 (m, 2H), 3.69-3.59 (m, 2H), 3.32-3.27 (m, 1H), 3.06-2.99 (m, 1H), 2.43-2.32 (m, 2H), 1.71-1.64 (m, 2H), 1.64-1.55 (m, 2H), 1.54-1.49 (m, 3H). MS m/z: 510.15 [M+H]⁺.

### Example 970: 8-(1-(2,2-difluoroethyl)-1H-pyrazolo[3,4-b]pyrazin-6-yl)-2-((2-methoxypyrimidin-4-yl)methyl)-2,8-diazaspiro[4.5]decan-3-one

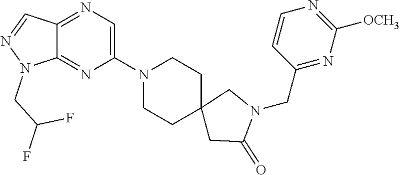 [SMILES: COc1nccc(CN2CC3(CCN(c4cnc5cnn(CC(F)F)c5n4)CC3)CC2=O)n1]

Step 1: (2-methoxypyrimidin-4-yl)methyl methanesulfonate: Followed the general procedure R using (2-methoxypyrimidin-4-yl)methanol (200 mg, 1.427 mmol, 1 equiv) and Ms₂O (296 mg, 1.71 mmol, 1.2 equiv.) as the starting materials to give (2-methoxypyrimidin-4-yl)methyl methanesulfonate (170 mg, 54%) as a white liquid. MS m/z: 219 [M+H]⁺.

Step 2: tert-butyl 2-((2-methoxypyrimidin-4-yl)methyl)-3-oxo-2,8-diazaspiro[4.5]decane-8-carboxylate: Followed the general procedure E using (2-methoxypyrimidin-4-yl)methyl methanesulfonate (170 mg, 0.779 mmol, 1 equiv) and tert-butyl 3-oxo-2,8-diazaspiro[4.5]decane-8-carboxylate (237 mg, 0.935 mmol, 1.2 equiv) as the starting materials to give tert-butyl 2-((2-methoxypyrimidin-4-yl)methyl)-3-oxo-2,8-diazaspiro[4.5]decane-8-carboxylate (200 mg, 68%) as a white solid. MS m/z: 377 [M+H]⁺.

Step 3: 2-((2-methoxypyrimidin-4-yl)methyl)-2,8-diazaspiro[4.5]decan-3-one hydrochloride: Followed the general procedure B using tert-butyl 2-[(2-methoxypyrimidin-4-yl)methyl]-3-oxo-2,8-diazaspiro[4.5]decane-8-carboxylate (200 mg, 0.531 mmol, 1 equiv) as the starting material to give the crude product 2-((2-methoxypyrimidin-4-yl)methyl)-2,8-diazaspiro[4.5]decan-3-one hydrochloride (70 mg) MS m/z: 277 [M+H]⁺.

Step 4: 8-(1-(2,2-difluoroethyl)-1H-pyrazolo[3,4-b]pyrazin-6-yl)-2-((2-methoxypyrimidin-4-yl)methyl)-2,8-diazaspiro[4.5]decan-3-one: Followed the general procedure I using 2-((2-methoxypyrimidin-4-yl)methyl)-2,8-diazaspiro[4.5]decan-3-one hydrochloride (70 mg, 0.253 mmol, 1 equiv) and 6-chloro-1-(2,2-difluoroethyl)-1H-pyrazolo[3,4-b]pyrazine (66.4 mg, 0.304 mmol, 1.2 equiv) as the starting materials to give 8-(1-(2,2-difluoroethyl)-1H-pyrazolo[3,4-b]pyrazin-6-yl)-2-((2-methoxypyrimidin-4-yl)methyl)-2,8-diazaspiro[4.5]decan-3-one (17.6 mg, 15%) as a white solid. ¹H NMR (400 MHz, DMSO-d₆) δ 8.56 (d, J=5.0 Hz, 1H), 8.47 (s, 1H), 8.12 (s, 1H), 7.01 (d, J=5.0 Hz, 1H), 6.43 (tt, J=55.0, 3.9 Hz, 1H), 4.69 (td, J=15.0, 3.8 Hz, 2H), 4.44 (s, 2H), 3.94-3.89 (m, 3H), 3.89-3.82 (m, 2H), 3.69 (dt, J=13.2, 5.6 Hz, 2H), 3.35-3.31 (m, 2H), 2.41-2.36 (m, 2H), 1.73-1.66 (m, 4H). MS m/z: 459.25 [M+H]⁺.

### Example 971: 8-(1-(2,2-difluoroethyl)-1H-pyrazolo[3,4-b]pyrazin-6-yl)-2-((2-(2,2,2-trifluoroethoxy)pyrimidin-4-yl)methyl)-2,8-diazaspiro[4.5]decan-3-one

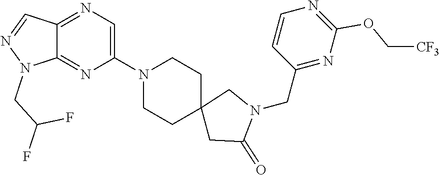 [SMILES: O=C1CC2(CCN(c3cnc4cnn(CC(F)F)c4n3)CC2)CN1Cc1ccnc(OCC(F)(F)F)n1]

Step 1: 4-methyl-2-(2,2,2-trifluoroethoxy)pyrimidine: Followed the general procedure E using 2-chloro-4-methylpyrimidine (1.00 g, 7.78 mmol, 1.0 equiv.) and trifluoroethanol (1.56 g, 15.5 mmol, 2.0 equiv.) as the starting materials to give 4-methyl-2-(2,2,2-trifluoroethoxy)pyrimidine (1.20 g, 76%) as a brown oil. MS m/z: 193 [M+H]⁺

Step 2: 4-(bromomethyl)-2-(2,2,2-trifluoroethoxy)pyrimidine: To a stirred solution of 4-methyl-2-(2,2,2-trifluoroethoxy)pyrimidine (1.2 g, 6.24 mmol, 1.0 equiv.) and AcOK (1.23 g, 12.5 mmol, 2.0 equiv.) in AcOH (8 mL) was added Br₂ (1.00 g, 6.24 mmol, 1.0 equiv.) dropwise at room temperature. The resulting mixture was stirred for 2 h at 80° C. The desired product was detected by LCMS. The mixture was allowed to cool down to room temperature and was basified to pH˜8 with saturated NaHCO₃ (aq.). The aqueous layer was extracted with CH₂Cl₂ (2×40 mL). The resulting mixture was concentrated under reduced pressure. The residue was purified by silica gel column chromatography, eluted with PE/EA (10:1) to afford crude product 4-(bromomethyl)-2-(2,2,2-trifluoroethoxy)pyrimidine (700 mg) as a brown oil. MS m/z: 271 [M+H]⁺.

Step 3: 8-(1-(2,2-difluoroethyl)-1H-pyrazolo[3,4-b]pyrazin-6-yl)-2-((2-(2,2,2-trifluoroethoxy)pyrimidin-4-yl)methyl)-2,8-diazaspiro[4.5]decan-3-one: A solution of 8-[1-(2,2-difluoroethyl)pyrazolo[3,4-b]pyrazin-6-yl]-2,8-diazaspiro[4.5]decan-3-one (74.4 mg, 0.221 mmol, 1 equiv) and NaH (60% w/w, 17.7 mg, 0.442 mmol, 2.00 equiv) in THF (5 mL) was stirred for 1 h at room temperature under N₂ atmosphere. To the above mixture was added 4-(bromomethyl)-2-(2,2,2-trifluoroethoxy)pyrimidine (700 mg, crude) in portions at 0° C. The resulting mixture was stirred for additional 2 h at room temperature. Desired product could be detected by LCMS. The mixture was quenched with saturated NH₄Cl aq., and extracted with EtOAc (2×20 mL). The combined organic layers was washed with brine, dried, filtered, evaporated, and purified by Combi-Flash to give 8-(1-(2,2-difluoroethyl)-1H-pyrazolo[3,4-b]pyrazin-6-yl)-2-((2-(2,2,2-trifluoroethoxy)pyrimidin-4-yl)methyl)-2,8-diazaspiro[4.5]decan-3-one (32.5 mg, 27.7%) as an off-white solid. ¹H NMR (300 MHz, DMSO-d₆) δ 8.64 (d, J=5.0 Hz, 1H), 8.48 (s, 1H), 8.13 (s, 1H), 7.16 (d, J=5.0 Hz, 1H), 6.42 (tt, J=55.4, 4.1 Hz, 1H), 5.04 (q, J=9.0 Hz, 2H), 4.69 (td, J=15.0, 3.8 Hz, 2H), 4.48 (s, 2H), 3.93-3.62 (m, 4H), 3.33 (s, 2H), 2.40 (s, 2H), 1.71 (t, J=5.6 Hz, 4H). MS m/z: 527.15 [M+H]⁺.

### Example 972: 8-(1-(2,2-difluoroethyl)-1H-pyrazolo[3,4-b]pyrazin-6-yl)-2-((3-methylisoxazol-5-yl)methyl)-2,8-diazaspiro[4.5]decan-3-one

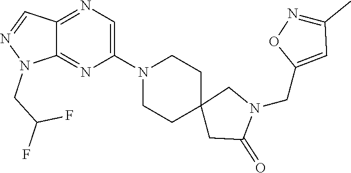 [SMILES: Cc1cc(CN2CC3(CCN(c4cnc5cnn(CC(F)F)c5n4)CC3)CC2=O)on1]

Step 1: 8-(1-(2,2-difluoroethyl)-1H-pyrazolo[3,4-b]pyrazin-6-yl)-2-((3-methylisoxazol-5-yl)methyl)-2,8-diazaspiro[4.5]decan-3-one: Followed the general procedure I using 8-(1-(2,2-difluoroethyl)-1H-pyrazolo[3,4-b]pyrazin-6-yl)-2,8-diazaspiro[4.5]decan-3-one (60 mg, 0.127 mmol, 1.0 equiv.) and 5-(bromomethyl)-3-methylisoxazole (100 mg, 0.152 mmol, 1.2 equiv.) as the starting materials to give 8-(1-(2,2-difluoroethyl)-1H-pyrazolo[3,4-b]pyrazin-6-yl)-2-((3-methylisoxazol-5-yl)methyl)-2,8-diazaspiro[4.5]decan-3-one (20.3 mg, 48%) as a white solid. ¹H NMR (400 MHz, DMSO-d₆) δ 8.47 (d, J=1.0 Hz, 1H), 8.12 (d, J=1.2 Hz, 1H), 6.72-6.03 (m, 2H), 4.75-4.63 (m, 2H), 4.53 (s, 2H), 3.91-3.83 (m, 2H), 3.70-3.62 (m, 2H), 3.42-3.31 (m, 2H), 2.34 (s, 2H), 2.22 (d, J=1.2 Hz, 3H), 1.65 (d, J=5.8 Hz, 4H). MS m/z: 432.05 [M+H]⁺.

### Example 973: 8-(1-(2,2-difluoroethyl)-1H-pyrazolo[3,4-b]pyrazin-6-yl)-2-(5-ethyl-2-(trifluoromethyl) thiazol-4-yl)-2,8-diazaspiro[4.5]decan-3-one

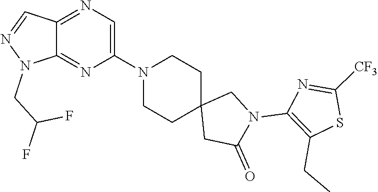 [SMILES: CCc1sc(C(F)(F)F)nc1N1CC2(CCN(c3cnc4cnn(CC(F)F)c4n3)CC2)CC1=O]

Step 1: N-methoxy-N-methyl-2-(trifluoromethyl)thiazole-5-carboxamide: Followed the general procedure G using 2-(trifluoromethyl)thiazole-5-carboxylic acid (550 mg, 2.79 mmol, 1.0 equiv.) and N,O-dimethylhydroxylamine (255 mg, 4.18 mmol, 1.5 equiv.) as the starting materials to give N-methoxy-N-methyl-2-(trifluoromethyl)thiazole-5-carboxamide (540 mg, 80%) as a yellow solid. MS m/z: 241 [M+H]⁺.

Step 2: 1-(2-(trifluoromethyl)thiazol-5-yl)ethan-1-one: Followed the general procedure P using N-methoxy-N-methyl-2-(trifluoromethyl)thiazole-5-carboxamide (540 mg, 2.25 mmol, 1.0 equiv.) as the starting material to give 1-(2-(trifluoromethyl)thiazol-5-yl)ethan-1-one (190 mg, 43%) as a yellow solid. MS m/z: 196 [M+H]⁺.

Step 3: 1-(2-(trifluoromethyl)thiazol-5-yl)ethan-1-ol: Followed the general procedure Q using 1-(2-(trifluoromethyl)thiazol-5-yl)ethan-1-one (190 mg, 0.974 mmol, 1.0 equiv.) as the starting material to give 1-(2-(trifluoromethyl)thiazol-5-yl)ethan-1-ol (130 mg, 67%) as a yellow oil. MS m/z: 198 [M+H]⁺.

Step 4: 1-(2-(trifluoromethyl)thiazol-5-yl)ethyl methanesulfonate: Followed the general procedure R using 1-(2-(trifluoromethyl)thiazol-5-yl)ethan-1-ol (130 mg, 0.659 mmol, 1.0 equiv.) and methanesulfonic anhydride (344 mg, 1.97 mmol, 3.0 equiv.) as the starting materials to give 1-(2-(trifluoromethyl)thiazol-5-yl)ethyl methanesulfonate (60 mg, 33%) as a yellow oil. MS m/z: 276 [M+H]⁺.

Step 5: 8-(1-(2,2-difluoroethyl)-1H-pyrazolo[3,4-b]pyrazin-6-yl)-2-(5-ethyl-2-(trifluoromethyl) thiazol-4-yl)-2,8-diazaspiro[4.5]decan-3-one: Followed the general procedure E using 1-(2-(trifluoromethyl)thiazol-5-yl)ethyl methanesulfonate (60 mg, 0.218 mmol, 1.0 equiv.) and 8-(1-(2,2-difluoroethyl)-1H-pyrazolo[3,4-b]pyrazin-6-yl)-2,8-diazaspiro[4.5]decan-3-one (73.3 mg, 0.218 mmol, 1.0 equiv.) as the starting materials to give 8-(1-(2,2-difluoroethyl)-1H-pyrazolo[3,4-b]pyrazin-6-yl)-2-(5-ethyl-2-(trifluoromethyl) thiazol-4-yl)-2,8-diazaspiro[4.5]decan-3-one (28.1 mg, 23%) as a yellow solid. ¹H NMR (300 MHz, DMSO-d₆) δ 8.48 (s, 1H), 8.12 (s, 1H), 6.62-6.23 (m, 1H), 4.74-4.63 (m, 2H), 3.96-3.88 (m, 2H), 3.76-3.68 (m, 4H), 2.80 (q, J=7.5 Hz, 2H), 2.54 (s, 2H), 1.76 (t, J=5.6 Hz, 4H), 1.24 (t, J=7.5 Hz, 3H). MS m/z: 516 [M+H]⁺.

### Example 974: 8-(1-(2,2-difluoroethyl)-1H-pyrazolo[3,4-b]pyrazin-6-yl)-2-((2-methyloxazol-5-yl)methyl)-2,8-diazaspiro[4.5]decan-3-one

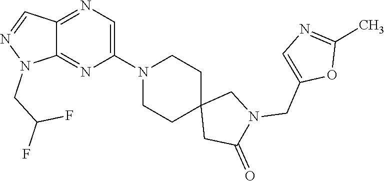 [SMILES: Cc1ncc(CN2CC3(CCN(c4cnc5cnn(CC(F)F)c5n4)CC3)CC2=O)o1]

Step 1: 8-(1-(2,2-difluoroethyl)-1H-pyrazolo[3,4-b]pyrazin-6-yl)-2-((2-methyloxazol-5-yl)methyl)-2,8-diazaspiro[4.5]decan-3-one: Followed the general procedure E using 8-(1-(2,2-difluoroethyl)-1H-pyrazolo[3,4-b]pyrazin-6-yl)-2,8-diazaspiro[4.5]decan-3-one (70 mg, 0.208 mmol, 1.0 equiv.) and 5-(chloromethyl)-2-methyloxazole (32 mg, 0.250 mmol, 1.2 equiv.) as the starting materials to give 8-(1-(2,2-difluoroethyl)-1H-pyrazolo[3,4-b]pyrazin-6-yl)-2-((2-methyloxazol-5-yl)methyl)-2,8-diazaspiro[4.5]decan-3-one (20 mg, 37%) as a yellow oil. ¹H NMR (400 MHz, DMSO-d₆) δ 8.47 (s, 1H), 8.13 (d, J=4.0 Hz, 1H), 6.93 (s, 1H), 6.57-6.30 (m, 1H), 4.68 (td, J=15.1, 3.9 Hz, 2H), 4.43 (s, 2H), 3.85 (dt, J=13.9, 5.3 Hz, 2H), 3.65 (dt, J=13.1, 5.8 Hz, 2H), 3.18 (s, 2H), 2.37 (s, 3H), 2.32 (s, 2H), 1.62 (t, J=5.6 Hz, 4H). MS m/z: 432.10 [M+H]⁺.

### Example 975: 8-(1-(2,2-difluoroethyl)-1H-pyrazolo[3,4-b]pyrazin-6-yl)-2-((5-methylthiazol-2-yl)methyl)-2,8-diazaspiro[4.5]decan-3-one

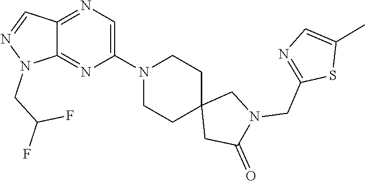 [SMILES: Cc1cnc(CN2CC3(CCN(c4cnc5cnn(CC(F)F)c5n4)CC3)CC2=O)s1]

Step 1: tert-butyl 2-((5-methylthiazol-2-yl)methyl)-3-oxo-2,8-diazaspiro[4.5]decane-8-carboxylate: Followed the general procedure I using tert-butyl 3-oxo-2,8-diazaspiro[4.5]decane-8-carboxylate (200 mg, 0.786 mmol, 1.0 equiv.) and 2-(chloromethyl)-5-methylthiazole (128 mg, 0.865 mmol, 1.1 equiv.) as the starting materials to give tert-butyl 2-((5-methylthiazol-2-yl)methyl)-3-oxo-2,8-diazaspiro[4.5]decane-8-carboxylate (120 mg, 41%) as a white solid. MS m/z: 366 [M+H]⁺.

Step 2: 2-((5-methylthiazol-2-yl)methyl)-2,8-diazaspiro[4.5]decan-3-one hydrochloride: Followed the general procedure B using tert-butyl 2-((5-methylthiazol-2-yl)methyl)-3-oxo-2,8-diazaspiro[4.5]decane-8-carboxylate (120 mg) as the starting material to give the crude product 2-((5-methylthiazol-2-yl)methyl)-2,8-diazaspiro[4.5]decan-3-one hydrochloride (80 mg) as a white solid. MS m/z: 266 [M+H]⁺.

Step 3: 8-(1-(2,2-difluoroethyl)-1H-pyrazolo[3,4-b]pyrazin-6-yl)-2-((5-methylthiazol-2-yl)methyl)-2,8-diazaspiro[4.5]decan-3-one: Followed the general procedure I using 2-((5-methylthiazol-2-yl)methyl)-2,8-diazaspiro[4.5]decan-3-one (80 mg, 0.301 mmol, 1.0 equiv.) and 6-chloro-1-(2,2-difluoroethyl)-1H-pyrazolo[3,4-b]pyrazine (72.5 mg, 0.331 mmol, 1.1 equiv.) as the starting materials to give 8-(1-(2,2-difluoroethyl)-1H-pyrazolo[3,4-b]pyrazin-6-yl)-2-((5-methylthiazol-2-yl)methyl)-2,8-diazaspiro[4.5]decan-3-one (28.5 mg, 20%) as a white solid. ¹H NMR (400 MHz, DMSO-d₆) δ 8.47 (s, 1H), 8.13 (s, 1H), 7.42 (s, 1H), 6.44 (tt, J=54.9, 3.8 Hz, 1H), 4.69 (td, J=15.0, 3.8 Hz, 2H), 4.62 (s, 2H), 3.92-3.82 (m, 2H), 3.73-3.62 (m, 2H), 3.29 (s, 2H), 2.43 (s, 3H), 2.36 (s, 2H), 1.66 (t, J=5.5 Hz, 4H). MS m/z: 448.05 [M+H]⁺.

### Example 976: 8-(1-(2,2-difluoroethyl)-1H-pyrazolo[3,4-b]pyrazin-6-yl)-2-((5-ethylthiazol-2-yl)methyl)-2,8-diazaspiro[4.5]decan-3-one

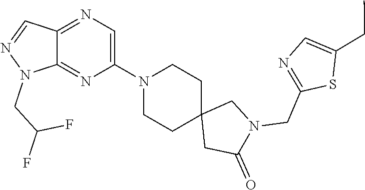 [SMILES: CCc1cnc(CN2CC3(CCN(c4cnc5cnn(CC(F)F)c5n4)CC3)CC2=O)s1]

Step 1: tert-butyl 2-((5-chlorothiazol-2-yl)methyl)-3-oxo-2,8-diazaspiro[4.5]decane-8-carboxylate: Followed the general procedure K using tert-butyl 2,8-diazaspiro[4.5]decane-8-carboxylate (300 mg, 1.18 mmol, 1.0 equiv.) and 5-chloro-2-(chloromethyl)thiazole (238 mg, 1.42 mmol, 1.2 equiv.) as the starting materials to give tert-butyl 2-((5-chlorothiazol-2-yl)methyl)-3-oxo-2,8-diazaspiro[4.5]decane-8-carboxylate (160 mg, 35%) as a white solid. MS m/z: 386 [M+H]⁺.

Step 2: tert-butyl 3-oxo-2-((5-vinylthiazol-2-yl)methyl)-2,8-diazaspiro[4.5]decane-8-carboxylate: Followed the general procedure L using 2-((5-chlorothiazol-2-yl)methyl)-3-oxo-2,8-diazaspiro[4.5]decane-8-carboxylate (160 mg, 0.415 mmol, 1.0 equiv.) and 2-ethenyl-4,4,5,5-tetramethyl-1,3,2-dioxaborolane (76 mg, 0.498 mmol, 1.2 equiv.) as the starting materials, Pd(dppf)Cl₂ (60 mg, 0.083 mmol, 0.2 equiv.) as the catalyst to give tert-butyl 3-oxo-2-((5-vinylthiazol-2-yl)methyl)-2,8-diazaspiro[4.5]decane-8-carboxylate (70 mg, 44%) as a white solid. MS m/z: 378 [M+H]⁺.

Step 3: tert-butyl 2-((5-ethylthiazol-2-yl)methyl)-3-oxo-2,8-diazaspiro[4.5]decane-8-carboxylate: Followed the general procedure U using tert-butyl 3-oxo-2-((5-vinylthiazol-2-yl)methyl)-2,8-diazaspiro[4.5]decane-8-carboxylate (70.0 mg, 0.185 mmol, 1.0 equiv.) as the starting material to give tert-butyl 2-((5-ethylthiazol-2-yl)methyl)-3-oxo-2,8-diazaspiro[4.5]decane-8-carboxylate (50 mg, 71%) as a white solid. MS m/z: 380 [M+H]⁺.

Step 4: 2-((5-ethylthiazol-2-yl)methyl)-2,8-diazaspiro[4.5]decan-3-one: Followed the general procedure H using tert-butyl 2-((5-ethylthiazol-2-yl)methyl)-3-oxo-2,8-diazaspiro[4.5]decane-8-carboxylate (50 mg) as the starting material to give the crude product 2-((5-ethylthiazol-2-yl)methyl)-2,8-diazaspiro[4.5]decan-3-one (40 mg). MS m/z: 280 [M+H]⁺.

Step 5: 8-(1-(2,2-difluoroethyl)-1H-pyrazolo[3,4-b]pyrazin-6-yl)-2-((5-ethylthiazol-2-yl)methyl)-2,8-diazaspiro[4.5]decan-3-one: Followed the general procedure I using 2-((5-ethylthiazol-2-yl)methyl)-2,8-diazaspiro[4.5]decan-3-one (40 mg, 142 μmol, 1.0 equiv.) and 6-chloro-1-(2,2-difluoroethyl)-1H-pyrazolo[3,4-b]pyrazine (32 mg, 142 μmol, 1.0 equiv.) as the starting materials to give 8-(1-(2,2-difluoroethyl)-1H-pyrazolo[3,4-b]pyrazin-6-yl)-2-((5-ethylthiazol-2-yl)methyl)-2,8-diazaspiro[4.5]decan-3-one (23 mg, 34%) as a white solid. ¹H NMR (400 MHz, Chloroform-d) δ 8.22 (s, 1H), 8.04 (s, 1H), 7.53 (s, 1H), 6.20 (tt, J=55.5, 4.4 Hz, 1H), 4.69-4.61 (m, 4H), 3.87 (d, J=13.7 Hz, 2H), 3.65-3.57 (m, 2H), 3.21 (s, 2H), 3.05 (q, J=7.4 Hz, 2H), 2.43 (s, 2H), 1.73 (s, 4H), 1.40 (t, J=7.4 Hz, 3H). MS m/z: 462.10 [M+H]⁺.

### Example 977: 3-(2,2-difluoroethyl)-1-methyl-5-(2-(2-(trifluoromethyl)pyridin-4-yl)-2,6-diazaspiro[3.4]octan-6-yl)-1,3-dihydro-2H-imidazo[4,5-b]pyrazin-2-one

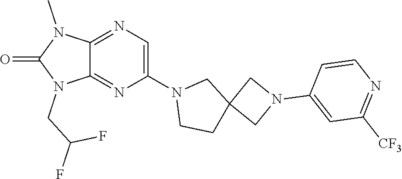 [SMILES: Cn1c(=O)n(CC(F)F)c2nc(N3CCC4(CN(c5ccnc(C(F)(F)F)c5)C4)C3)cnc21]

Step 1: 5-bromo-1,3-dihydro-2H-imidazo[4,5-b]pyrazin-2-one: To a stirred solution of 5-bromopyrazine-2,3-diamine (1.00 g, 5.31 mmol, 1.0 equiv.) and di(1H-imidazol-1-yl)methanone (1.29 g, 7.96 mmol, 1.5 equiv.) in THF (15.0 mL). The resulting mixture was stirred for overnight at 60° C. The reaction mixture was diluted with water (50 mL), extracted with EtOAc (3×80 mL). The combined organic layers were washed with brine (50 mL), dried over anhydrous Na₂SO₄. After filtration, the filtrate was concentrated to dryness under reduced pressure. The residue was purified by silica gel column chromatography, eluted with DCM/MeOH (0-30% 20 min) to afford 5-bromo-1,3-dihydro-2H-imidazo[4,5-b]pyrazin-2-one (600 mg) as a white solid. MS m/z: 215 [M+H]⁺.

Step 2: 6-bromo-1-(2,2-difluoroethyl)-1,3-dihydro-2H-imidazo[4,5-b]pyrazin-2-one: Followed the general procedure K using 5-bromo-1,3-dihydro-2H-imidazo[4,5-b]pyrazin-2-one (600 mg, 2.80 mmol, 1.0 equiv.) and 2,2-difluoroethyl trifluoromethanesulfonate (600 mg, 2.80 mmol, 1.0 equiv.) as the starting material to give 6-bromo-1-(2,2-difluoroethyl)-1,3-dihydro-2H-imidazo[4,5-b]pyrazin-2-one (120 mg, 15%) as a white solid. MS m/z: 279 [M+H]⁺.

Step 3: 5-bromo-3-(2,2-difluoroethyl)-1-methyl-1,3-dihydro-2H-imidazo[4,5-b]pyrazin-2-one: Followed the general procedure K using 6-bromo-1-(2,2-difluoroethyl)-1,3-dihydro-2H-imidazo[4,5-b]pyrazin-2-one (120 mg, 0.431 mmol, 1.0 equiv) and iodomethane (92 mg, 0.646 mmol, 1.5 equiv) as the starting materials to give 5-bromo-3-(2,2-difluoroethyl)-1-methyl-1,3-dihydro-2H-imidazo[4,5-b]pyrazin-2-one (90 mg, 71%) as a yellow oil. MS m/z: 293 [M+H]⁺.

Step 4: 3-(2,2-difluoroethyl)-1-methyl-5-(2-(2-(trifluoromethyl)pyridin-4-yl)-2,6-diazaspiro[3.4]octan-6-yl)-1,3-dihydro-2H-imidazo[4,5-b]pyrazin-2-one: Followed the general procedure Y using 5-bromo-3-(2,2-difluoroethyl)-1-methyl-1,3-dihydro-2H-imidazo[4,5-b]pyrazin-2-one (90 mg, 0.308 mmol, 1.0 equiv.) and 2-(2-(trifluoromethyl) pyridin-4-yl)-2,6-diazaspiro[3.4]octane hydrochloride (99.3 mg, 0.338 mmol, 1.1 equiv.) as the starting materials to give 3-(2,2-difluoroethyl)-1-methyl-5-(2-(2-(trifluoromethyl)pyridin-4-yl)-2,6-diazaspiro[3.4]octan-6-yl)-1,3-dihydro-2H-imidazo[4,5-b]pyrazin-2-one (52.6 mg, 52%) as a white solid. ¹H NMR (300 MHz, DMSO-d₆) δ 8.24 (d, J=5.7 Hz, 1H), 7.28 (s, 1H), 6.75 (d, J=2.2 Hz, 1H), 6.59-6.19 (m, 2H), 4.27-4.15 (m, 2H), 4.06-3.99 (m, 4H), 3.67 (s, 2H), 3.46 (t, J=6.8 Hz, 2H), 3.31 (s, 3H), 2.27 (t, J=6.8 Hz, 2H). MS m/z: 470.1 [M+H]⁺.

### Example 978: 1-methyl-3-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one; Example 979: 3-methyl-1-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one

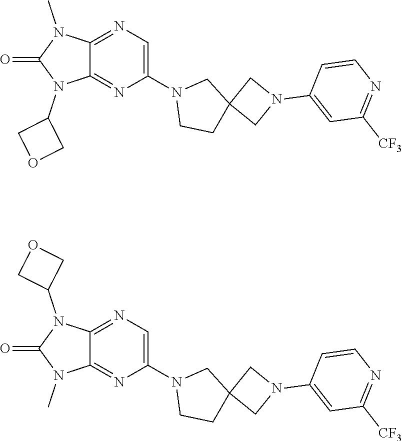 [SMILES: Cn1c(=O)n(C2COC2)c2nc(N3CCC4(CN(c5ccnc(C(F)(F)F)c5)C4)C3)cnc21 ; Cn1c(=O)n(C2COC2)c2ncc(N3CCC4(CN(c5ccnc(C(F)(F)F)c5)C4)C3)nc21]

Step 1: 5-bromo-1-methyl-1,3-dihydro-2H-imidazo[4,5-b]pyrazin-2-one; 6-bromo-1-methyl-1,3-dihydro-2H-imidazo[4,5-b]pyrazin-2-one: Followed the general procedure K using 5-bromo-1,3-dihydro-2H-imidazo[4,5-b]pyrazin-2-one (550 mg, 2.33 mmol, 1.0 equiv.) and iodomethane (165 mg, 1.16 mmol, 0.5 equiv.) as the starting materials to give 5-bromo-1-methyl-1,3-dihydro-2H-imidazo[4,5-b]pyrazin-2-one and 6-bromo-1-methyl-1,3-dihydro-2H-imidazo[4,5-b]pyrazin-2-one two isomers (120 mg, 22%) as a yellow solid. MS m/z: 229 M

Step 2: 5-bromo-1-methyl-3-(oxetan-3-yl)-1,3-dihydro-2H-imidazo[4,5-b]pyrazin-2-one: 5-bromo-3-methyl-1-(oxetan-3-yl)-1,3-dihydro-2H-imidazo[4,5-b]pyrazin-2-one: Followed the general procedure K using 5-bromo-1-methyl-1,3-dihydro-2H-imidazo[4,5-b]pyrazin-2-one and 6-bromo-1-methyl-1,3-dihydro-2H-imidazo[4,5-b]pyrazin-2-one two isomers (120 mg, 0.526 mmol, 1.0 equiv.) and oxetan-3-yl trifluoromethanesulfonate (130 mg, 0.631 mmol, 1.2 equiv.) as the starting materials to give 5-bromo-1-methyl-3-(oxetan-3-yl)-1,3-dihydro-2H-imidazo[4,5-b]pyrazin-2-one and 5-bromo-3-methyl-1-(oxetan-3-yl)-1,3-dihydro-2H-imidazo[4,5-b]pyrazin-2-one two isomers (60 mg, 40%) as a yellow oil. MS m/z: 285 [M+H]⁺.

Step 3: 1-methyl-3-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one: 3-methyl-1-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one: Followed the general procedure Y using 5-bromo-1-methyl-3-(oxetan-3-yl)-1,3-dihydro-2H-imidazo[4,5-b]pyrazin-2-one and 5-bromo-3-methyl-1-(oxetan-3-yl)-1,3-dihydro-2H-imidazo[4,5-b]pyrazin-2-one two isomers (60 mg, 0.211 mmol, 1.0 equiv.) and 2-(2-(trifluoromethyl)pyridin-4-yl)-2,6-diazaspiro[3.4]octane hydrochloride (68 mg, 0.232 mmol, 1.1 equiv.) as the starting materials to give the crude product. The crude product was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 24% B to 34% B in 8 min, 34% B; Wave Length: 254/220 nm; RT1(min): 8.63; Number Of Runs: 0) to afford 1-methyl-3-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one (6.6 mg, 7%) as a white solid, and 3-methyl-1-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one (14 mg, 14%) as a white solid.

### Example 980: 3-(2,2-difluoroethyl)-1-ethyl-5-(2-(2-(trifluoromethyl)pyridin-4-yl)-2,6-diazaspiro[3.4]octan-6-yl)-1,3-dihydro-2H-imidazo[4,5-b]pyrazin-2-one

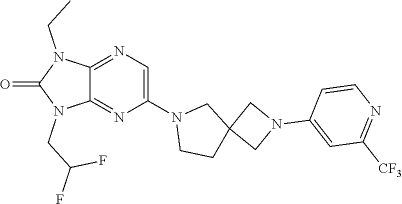 [SMILES: CCn1c(=O)n(CC(F)F)c2nc(N3CCC4(CN(c5ccnc(C(F)(F)F)c5)C4)C3)cnc21]

Step 1: 5-bromo-3-(2,2-difluoroethyl)-1-ethyl-1,3-dihydro-2H-imidazo[4,5-b]pyrazin-2-one: Followed the general procedure K using 6-bromo-1-(2,2-difluoroethyl)-1,3-dihydro-2H-imidazo[4,5-b]pyrazin-2-one (100 mg, 0.358 mmol, 1.0 equiv.) and iodoethane (83.8 mg, 0.537 mmol, 1.5 equiv.) as the starting materials to give 5-bromo-3-(2,2-difluoroethyl)-1-ethyl-1,3-dihydro-2H-imidazo[4,5-b]pyrazin-2-one (60 mg, 54%) as a yellow oil. MS m/z: 307 [M+H]⁺

Step 2: 3-(2,2-difluoroethyl)-1-ethyl-5-(2-(2-(trifluoromethyl)pyridin-4-yl)-2,6-diazaspiro[3.4]octan-6-yl)-1,3-dihydro-2H-imidazo[4,5-b]pyrazin-2-one: Followed the general procedure Y using 5-bromo-3-(2,2-difluoroethyl)-1-ethyl-1,3-dihydro-2H-imidazo[4,5-b]pyrazin-2-one (60 mg, 0.195 mmol, 1.0 equiv.) and 2-(2-(trifluoromethyl)pyridin-4-yl)-2,6-diazaspiro[3.4]octane hydrochloride (68.9 mg, 0.195 mmol, 1.0 equiv.) as the starting materials to give 3-(2,2-difluoroethyl)-1-ethyl-5-(2-(2-(trifluoromethyl)pyridin-4-yl)-2,6-diazaspiro[3.4]octan-6-yl)-1,3-dihydro-2H-imidazo[4,5-b]pyrazine-2-one (25.6 mg, 30%) as a white solid. ¹H NMR (300 MHz, DMSO-d₆) δ 8.25 (d, J=5.7 Hz, 1H), 7.29 (s, 1H), 6.77 (d, J=3.0 Hz, 1H), 6.78-6.22 (m, 2H), 4.27-4.15 (m, 2H), 4.03 (t, J=3.3 Hz, 4H), 3.86 (q, J=7.2 Hz, 2H), 3.67 (s, 2H), 3.47 (d, J=6.5 Hz, 2H), 2.28 (t, J=6.9 Hz, 2H), 1.26 (t, J=7.1 Hz, 3H). MS m/z: 470.1 [M+H]⁺.

### Example 981: 1-ethyl-3-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one; Example 982: 3-ethyl-1-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one

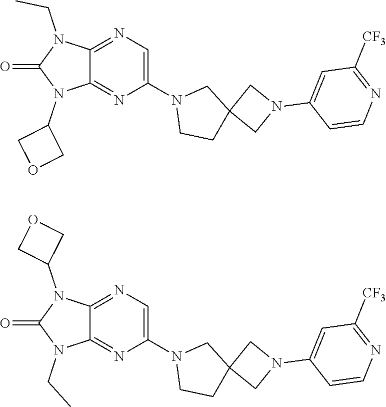 [SMILES: CCn1c(=O)n(C2COC2)c2nc(N3CCC4(CN(c5ccnc(C(F)(F)F)c5)C4)C3)cnc21 ; CCn1c(=O)n(C2COC2)c2ncc(N3CCC4(CN(c5ccnc(C(F)(F)F)c5)C4)C3)nc21]

Step 1: 5-bromo-1-ethyl-1,3-dihydro-2H-imidazo[4,5-b]pyrazin-2-one; 6-bromo-1-ethyl-1,3-dihydro-2H-imidazo[4,5-b]pyrazin-2-one: Followed the general procedure K using 5-bromo-1,3-dihydro-2H-imidazo[4,5-b]pyrazin-2-one (220 mg, 1.02 mmol, 1.0 equiv.) and iodoethane (80.1 mg, 0.51 mmol, 0.5 equiv.) as the starting materials to give 5-bromo-1-ethyl-1,3-dihydro-2H-imidazo[4,5-b]pyrazin-2-one and 6-bromo-1-ethyl-1,3-dihydro-2H-imidazo[4,5-b]pyrazin-2-one two isomers (80 mg, 32%) as a yellow solid. MS m/z: 243 [M+H]⁺.

Step 2: 5-bromo-1-ethyl-3-(oxetan-3-yl)-1,3-dihydro-2H-imidazo[4,5-b]pyrazin-2-one: 5-bromo-3-ethyl-1-(oxetan-3-yl)-1,3-dihydro-2H-imidazo[4,5-b]pyrazin-2-one: Followed the general procedure K using 5-bromo-1-ethyl-1,3-dihydro-2H-imidazo[4,5-b]pyrazin-2-one and 6-bromo-1-ethyl-1,3-dihydro-2H-imidazo[4,5-b]pyrazin-2-one two isomers (80 mg, 0.330 mmol, 1.0 equiv.) and oxetan-3-yl trifluoromethanesulfonate (81.7 mg, 0.396 mmol, 1.2 equiv.) as the starting materials to give 5-bromo-1-ethyl-3-(oxetan-3-yl)-1,3-dihydro-2H-imidazo[4,5-b]pyrazin-2-one and 5-bromo-3-ethyl-1-(oxetan-3-yl)-1,3-dihydro-2H-imidazo[4,5-b]pyrazin-2-one two isomers (60 mg, 60%) as a yellow oil. MS m/z: 299 [M+H]⁺.

Step 3: 1-ethyl-3-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one: 3-ethyl-1-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one: Followed the general procedure Y using 5-bromo-1-ethyl-3-(oxetan-3-yl)-1,3-dihydro-2H-imidazo[4,5-b]pyrazin-2-one and 5-bromo-3-ethyl-1-(oxetan-3-yl)-1,3-dihydro-2H-imidazo[4,5-b]pyrazin-2-one two isomers (60 mg, 0.201 mmol, 1.0 equiv.) and 2-(2-(trifluoromethyl)pyridin-4-yl)-2,6-diazaspiro[3.4]octane hydrochloride (59 mg, 0.201 mmol, 1.0 equiv.) as the starting materials to give the crude product. The crude product was purified by Prep-HPLC to afford 1-ethyl-3-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one (5.4 mg, 8.4%) as a white solid, and 3-ethyl-1-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one (9.5 mg, 14%) as a white solid.

1-ethyl-3-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one ¹H NMR (400 MHz, Chloroform-d) δ 8.35 (s, 1H), 7.22 (s, 1H), 6.64 (s, 1H), 6.44 (s, 1H), 5.64 (q, J=7.7 Hz, 1H), 5.54 (t, J=6.7 Hz, 2H), 4.95-4.91 (m, 2H), 4.17-4.10 (m, 4H), 3.97 (q, J=7.2 Hz, 2H), 3.79 (s, 2H), 3.60 (t, J=6.7 Hz, 2H), 2.39 (t, J=6.9 Hz, 2H), 1.36 (t, J=7.2 Hz, 3H). MS m/z: 476.15 [M+H]⁺.

3-ethyl-1-(oxetan-3-yl)-5-(2-(2-(trifluoromethyl)pyridin-4-yl)-2,6-diazaspiro[3.4]octan-6-yl)-1,3-dihydro-2H-imidazo[4,5-b]pyrazin-2-one ¹H NMR (400 MHz, Chloroform-d) δ 8.36 (s, 1H), 7.24 (s, 1H), 6.65 (s, 1H), 6.49 (s, 1H), 5.63 (t, J=7.6 Hz, 1H), 5.46 (t, J=6.7 Hz, 2H), 4.98-4.94 (m, 2H), 4.20 (s, 4H), 3.96 (d, J=7.0 Hz, 2H), 3.79 (s, 2H), 3.60 (s, 2H), 2.40 (s, 2H), 1.37 (t, J=7.1 Hz, 3H). MS m/z: 476.15 [M+H]⁺.

### Example 983: 1-ethyl-8-(1-(oxetan-3-yl)-1H-pyrazolo[3,4-b]pyrazin-6-yl)-3-(2-(trifluoromethyl)pyridin-4-yl)-1,3,8-triazaspiro[4.5]decane-2,4-dione

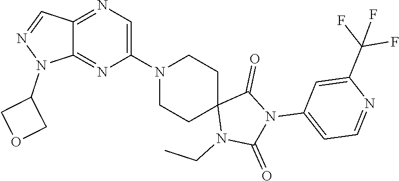 [SMILES: CCN1C(=O)N(c2ccnc(C(F)(F)F)c2)C(=O)C12CCN(c1cnc3cnn(C4COC4)c3n1)CC2]

1-ethyl-8-(1-(oxetan-3-yl)-1H-pyrazolo[3,4-b]pyrazin-6-yl)-3-(2-(trifluoromethyl)pyridin-4-yl)-1,3,8-triazaspiro[4.5]decane-2,4-dione: Followed the general procedure I using 1-ethyl-3-(2-(trifluoromethyl)pyridin-4-yl)-1,3,8-triazaspiro[4.5]decane-2,4-dione (80 mg, 0.234 mmol, 1 equiv) and 6-chloro-1-(oxetan-3-yl)-1H-pyrazolo[3,4-b]pyrazine (26 mg, 0.122 mmol, 1.2 equiv) as the starting materials to give 1-ethyl-8-(1-(oxetan-3-yl)-1H-pyrazolo[3,4-b]pyrazin-6-yl)-3-(2-(trifluoromethyl)pyridin-4-yl)-1,3,8-triazaspiro[4.5]decane-2,4-dione (20 mg, 51%) as a white solid. ¹H NMR (400 MHz, DMSO-d₆) δ 8.90 (d, J=5.4 Hz, 1H), 8.52 (s, 1H), 8.21 (s, 1H), 8.18 (d, J=1.9 Hz, 1H), 7.98 (dd, J=5.4, 1.9 Hz, 1H), 5.99-5.87 (m, 1H), 5.07 (t, J=6.4 Hz, 2H), 5.02-4.94 (m, 2H), 4.67-4.59 (m, 2H), 3.68-3.56 (m, 2H), 3.37-3.32 (m, 2H), 2.19-2.02 (m, 4H), 1.15 (t, J=7.0 Hz, 3H). MS m/z: 517.15 [M+H]⁺.

### Example 984: 1-ethyl-8-(3-methyl-1-(oxetan-3-yl)-1H-pyrazolo[3,4-b]pyrazin-6-yl)-3-(2-(trifluoromethyl)pyridin-4-yl)-1,3,8-triazaspiro[4.5]decane-2,4-dione

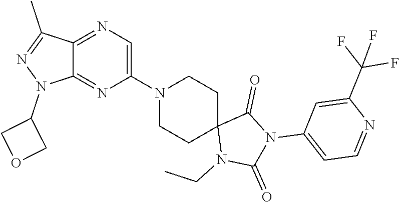 [SMILES: CCN1C(=O)N(c2ccnc(C(F)(F)F)c2)C(=O)C12CCN(c1cnc3c(C)nn(C4COC4)c3n1)CC2]

Step 1: tert-butyl 2,4-dioxo-3-(2-(trifluoromethyl)pyridin-4-yl)-1,3,8-triazaspiro[4.5]decane-8-carboxylate: A solution of 4-(((benzyloxy)carbonyl)amino)-1-(tert-butoxycarbonyl)piperidine-4-carboxylic acid (1 g, 2.64 mmol, 1.0 equiv.), 2-(trifluoromethyl)pyridin-4-amine (0.51 g, 3.17 mmol, 1.2 equiv.), TCFH (1.11 g, 3.96 mmol, 1.5 equiv.) and NMI (0.65 g, 7.93 mmol, 3.0 equiv) in ACN (15 mL) was stirred for overnight at 60° C. The resulting mixture was concentrated under vacuum. The residue was purified by reversed-phase flash chromatography with the following conditions: column, C18 silica gel; mobile phase, MeCN in Water (0.1% NH₃·H₂O), 10% to 100% gradient in 30 min; detector, UV 254 nm. This resulted in tert-butyl 2,4-dioxo-3-(2-(trifluoromethyl)pyridin-4-yl)-1,3,8-triazaspiro[4.5]decane-8-carboxylate (200 mg, 18%) as a white solid. MS m/z: 415 [M+H]⁺.

Step 2: tert-butyl 1-ethyl-2,4-dioxo-3-(2-(trifluoromethyl)pyridin-4-yl)-1,3,8-triazaspiro[4.5]decane-8-carboxylate: Followed the general procedure K using tert-butyl 2,4-dioxo-3-(2-(trifluoromethyl)pyridin-4-yl)-1,3,8-triazaspiro[4.5]decane-8-carboxylate (200 mg, 0.48 mmol, 1.0 equiv.) and EtI (150 mg, 0.96 mmol, 2.0 equiv.) as the starting materials to give tert-butyl 1-ethyl-2,4-dioxo-3-(2-(trifluoromethyl)pyridin-4-yl)-1,3,8-triazaspiro[4.5]decane-8-carboxylate (150 mg, 70%) as a white solid. MS m/z: 443 [M+H]⁺.

Step 3: 1-ethyl-3-(2-(trifluoromethyl)pyridin-4-yl)-1,3,8-triazaspiro[4.5]decane-2,4-dione hydrochloride: Followed the general procedure B using tert-butyl 1-ethyl-2,4-dioxo-3-(2-(trifluoromethyl)pyridin-4-yl)-1,3,8-triazaspiro[4.5]decane-8-carboxylate (150 mg) as the starting material to give the crude product 1-ethyl-3-(2-(trifluoromethyl)pyridin-4-yl)-1,3,8-triazaspiro[4.5]decane-2,4-dione hydrochloride (120 mg). MS m/z: 343 [M+H]⁺.

Step 4: 1-ethyl-8-(3-methyl-1-(oxetan-3-yl)-1H-pyrazolo[3,4-b]pyrazin-6-yl)-3-(2-(trifluoromethyl)pyridin-4-yl)-1,3,8-triazaspiro[4.5]decane-2,4-dione: Followed the general procedure I using 1-ethyl-3-(2-(trifluoromethyl)pyridin-4-yl)-1,3,8-triazaspiro[4.5]decane-2,4-dione hydrochloride (50 mg, 0.145 mmol, 1.0 equiv.) and 6-chloro-3-methyl-1-(oxetan-3-yl)-1H-pyrazolo[3,4-b]pyrazine (35 mg, 0.16 mmol, 1.1 equiv.) as the starting materials to give 1-ethyl-8-(3-methyl-1-(oxetan-3-yl)-1H-pyrazolo[3,4-b]pyrazin-6-yl)-3-(2-(trifluoromethyl)pyridin-4-yl)-1,3,8-triazaspiro[4.5]decane-2,4-dione (28 mg, 38%) as a white solid. ¹H NMR (300 MHz, DMSO-d₆) δ 8.91 (d, J=5.4 Hz, 1H), 8.44 (s, 1H), 8.18 (d, J=1.9 Hz, 1H), 7.98 (dd, J=5.4, 1.9 Hz, 1H), 5.97-5.78 (m, 1H), 5.16-4.85 (m, 4H), 4.62 (d, J=13.5 Hz, 2H), 3.61 (t, J=12.0 Hz, 2H), 3.33-3.27 (m, 2H), 2.48 (s, 3H), 2.20-2.00 (m, 4H), 1.15 (t, J=7.0 Hz, 3H). MS m/z: 531.15 [M+H]⁺.

### Example 985: 1-(2,2-difluoroethyl)-3-methyl-6-(2-(2-(trifluoromethyl)pyridin-4-yl)-2,6-diazaspiro[3.4]octan-6-yl)-1H-pyrazolo[3,4-b]pyrazine

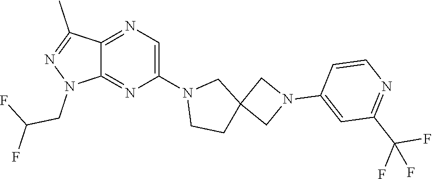 [SMILES: Cc1nn(CC(F)F)c2nc(N3CCC4(CN(c5ccnc(C(F)(F)F)c5)C4)C3)cnc12]

Step 1: tert-butyl 2-(2-(trifluoromethyl)pyridin-4-yl)-2,6-diazaspiro[3.4]octane-6-carboxylate: Followed the general procedure Y using tert-butyl 2,6-diazaspiro[3.4]octane-6-carboxylate hydrochloride (26 g, 104 mmol, 1.0 equiv.) and 4-bromo-2-(trifluoromethyl)pyridine (35.4 g, 156 mmol, 1.5 equiv.), as the starting materials to give tert-butyl 2-(2-(trifluoromethyl)pyridin-4-yl)-2,6-diazaspiro[3.4]octane-6-carboxylate (34 g, 90%) as a yellow solid. MS m/z: 358 [M+H]⁺.

Step 2: 2-(2-(trifluoromethyl)pyridin-4-yl)-2,6-diazaspiro[3.4]octane hydrochloride: Followed the general procedure B using tert-butyl 2-(2-(trifluoromethyl)pyridin-4-yl)-2,6-diazaspiro[3.4]octane-6-carboxylate (34 g) as the starting material to give the crude product 2-(2-(trifluoromethyl)pyridin-4-yl)-2,6-diazaspiro[3.4]octane hydrochloride (34 g). MS m/z: 258 [M+H]⁺.

Step 3: 6-chloro-3-iodo-1H-pyrazolo[3,4-b]pyrazine: To a stirred solution of 6-chloro-1H-pyrazolo[3,4-b]pyrazine (25 g, 162 mmol, 1.0 equiv.) in DMF (125 mL) was added NIS (47.4 g, 210 mmol, 1.3 equiv.) and TFA (16.2 mL, 0.1 equiv.) at room temperature. The resulting mixture was stirred for 2 h at 80° C. The resulting mixture was then pured in to 10% Na₂SO₃ aq. at 0° C., stirred for 1 h, and filtered. The filtrate was triturated with H₂O:ACN=2:1 (150 mL) to afford the product 6-chloro-3-iodo-1H-pyrazolo[3,4-b]pyrazine (38.9 g, 88%) as a yellow solid. MS m/z: 281 [M+H]⁺.

Step 4: 3-iodo-6-(2-(2-(trifluoromethyl)pyridin-4-yl)-2,6-diazaspiro[3.4]octan-6-yl)-1H-pyrazolo[3,4-b]pyrazine: Followed the general procedure I using 2-(2-(trifluoromethyl)pyridin-4-yl)-2,6-diazaspiro[3.4]octane hydrochloride (34 g, 132 mmol, 1.0 equiv.) and 6-chloro-3-iodo-1H-pyrazolo[3,4-b]pyrazine (31.9 g, 158 mmol, 1.2 equiv.) as the starting materials to give 3-iodo-6-(2-(2-(trifluoromethyl)pyridin-4-yl)-2,6-diazaspiro[3.4]octan-6-yl)-1H-pyrazolo[3,4-b]pyrazine (34 g, 73%) as yellow solid. MS m/z: 502 [M+H]⁺.

Step 5: 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine: Followed the general procedure K using 3-iodo-6-(2-(2-(trifluoromethyl)pyridin-4-yl)-2,6-diazaspiro[3.4]octan-6-yl)-1H-pyrazolo[3,4-b]pyrazine (25 g, 49.9 mmol, 1.0 equiv.) and 2,2-difluoroethyl trifluoromethanesulfonate (16.0 g, 74.8 mmol, 1.5 equiv.) as the starting materials to give 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine (20 g, 72%) as yellow solid. MS m/z: 566 [M+H]⁺.

Step 6: 1-(2,2-difluoroethyl)-3-methyl-6-(2-(2-(trifluoromethyl)pyridin-4-yl)-2,6-diazaspiro[3.4]octan-6-yl)-1H-pyrazolo[3,4-b]pyrazine: A solution of 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine (20 g, 35.3 mmol, 1.0 equiv.) and methylboronic acid (10.6 g, 176.9 mmol, 5.0 equiv.) and Pd(dppf)Cl₂·CH₂Cl₂ (1.44 g, 1.76 mmol, 0.05 equiv.) and K₃PO₄ (22.5 g, 106 mmol, 3.0 equiv.) in dioxane (200 mL) was stirred for 2 h at 80° C. under nitrogen atmosphere. Desired product could be detected by LCMS. The reaction mixture was filtrated and concentrated. The residue was purified by silica gel column chromatography eluted with EtOAc/PE=7/3 to afford impure product (9.1 g). The impure product (9.1 g) was desolved in EtOH (63 mL) at 80° C. To the resulting solution, charcoal (2.7 g, 30% w) was added, and the mixture was allowed to reflux for 2 h. After filtration, the filtrate was slowly cooled to 0° C. to crystalize the pure compound. The precipitate was collected, washed with cold EtOH (10 mL) and dried under rotary evaporator to affored 1-(2,2-difluoroethyl)-3-methyl-6-(2-(2-(trifluoromethyl)pyridin-4-yl)-2,6-diazaspiro[3.4]octan-6-yl)-1H-pyrazolo[3,4-b]pyrazine (8 g, 49.8%) as an white solid. ¹H NMR (400 MHz, DMSO-d₆) δ 8.25 (d, 1H), 8.02 (s, 1H), 6.76 (d, 1H), 6.66-6.24 (m, 2H), 4.59 (td, 2H), 4.10-3.99 (m, 4H), 3.85 (s, 2H), 3.66 (t, 2H), 2.42 (s, 3H), 2.31 (t, 2H). MS m/z: 454.00 [M+H]⁺.

### Example 986: 2-(2-(2-(2-(trifluoromethyl)pyridin-4-yl)-2,6-diazaspiro[3.4]octan-6-yl)pyrimidin-4-yl)-1,3,4-thiadiazole

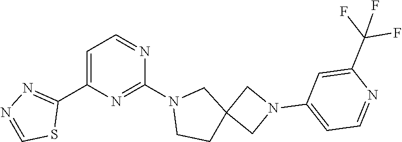 [SMILES: FC(F)(F)c1cc(N2CC3(CCN(c4nccc(-c5nncs5)n4)C3)C2)ccn1]

Step 1: methyl 2-(2-(2-(trifluoromethyl)pyridin-4-yl)-2,6-diazaspiro[3.4]octan-6-yl)pyrimidine-4-carboxylate: Followed the general procedure I using methyl 2-chloropyrimidine-4-carboxylate (300 mg, 1.73 mmol, 1.0 equiv.) and 2-(2-(trifluoromethyl)pyridin-4-yl)-2,6-diazaspiro[3.4]octane (493 mg, 1.90 μmol, 1.1 equiv.) as the starting materials to give methyl 2-(2-(2-(trifluoromethyl)pyridin-4-yl)-2,6-diazaspiro[3.4]octan-6-yl)pyrimidine-4-carboxylate (300 mg, 44%) as a white solid. MS m/z: 394 [M+H]⁺.

Step 2: 2-(2-(2-(trifluoromethyl)pyridin-4-yl)-2,6-diazaspiro[3.4]octan-6-yl)pyrimidine-4-carbohydrazide: Followed the general procedure S using methyl 2-(2-(2-(trifluoromethyl)pyridin-4-yl)-2,6-diazaspiro[3.4]octan-6-yl)pyrimidine-4-carboxylate (300 mg, 761 μmol, 1.0 equiv.) and hydrazine hydrate (39 mg, 961 μmol, 1.0 equiv.) as the starting materials to give 2-(2-(2-(trifluoromethyl)pyridin-4-yl)-2,6-diazaspiro[3.4]octan-6-yl)pyrimidine-4-carbohydrazide (250 mg, 83%) as a white solid. MS m/z: 394 [M+H]⁺.

Step 3: N′-formyl-2-(2-(2-(trifluoromethyl)pyridin-4-yl)-2,6-diazaspiro[3.4]octan-6-yl)pyrimidine-4-carbohydrazide: Followed the general procedure AF using 2-(2-(2-(trifluoromethyl)pyridin-4-yl)-2,6-diazaspiro[3.4]octan-6-yl)pyrimidine-4-carbohydrazide (250 mg, 634 μmol, 1.0 equiv.) as the starting material to give N′-formyl-2-(2-(2-(trifluoromethyl)pyridin-4-yl)-2,6-diazaspiro[3.4]octan-6-yl)pyrimidine-4-carbohydrazide (200 mg, 74%) as a colorless oil. MS m/z: 422 [M+H]⁺.

Step 4: 2-(2-(2-(2-(trifluoromethyl)pyridin-4-yl)-2,6-diazaspiro[3.4]octan-6-yl)pyrimidin-4-yl)-1,3,4-thiadiazole: Followed the general procedure T using N′-formyl-2-(2-(2-(trifluoromethyl)pyridin-4-yl)-2,6-diazaspiro[3.4]octan-6-yl)pyrimidine-4-carbohydrazide (200 mg, 473 μmol, 1.0 equiv.) as the starting material to give 2-(2-(2-(2-(trifluoromethyl)pyridin-4-yl)-2,6-diazaspiro[3.4]octan-6-yl)pyrimidin-4-yl)-1,3,4-thiadiazole (65.4 mg, 32%) as a white solid. ¹H NMR (400 MHz, DMSO-d₆) δ 9.76 (s, 1H), 8.61 (d, J=4.9 Hz, 1H), 8.25 (d, J=5.7 Hz, 1H), 7.42 (d, J=4.9 Hz, 1H), 6.75 (d, J=2.3 Hz, 1H), 6.57 (dd, J=5.7, 2.3 Hz, 1H), 4.07 (s, 2H), 4.01 (d, J=8.3 Hz, 2H), 3.82 (s, 2H), 3.64 (t, J=6.9 Hz, 2H), 2.29 (t, J=6.9 Hz, 2H). MS m/z: 420.05 [M+H]⁺.

### Example 987: 1-(oxetan-3-yl)-6-(2-(2-(trifluoromethyl)pyridin-4-yl)-2,6-diazaspiro[3.4]octan-6-yl)-1H-pyrazolo[3,4-b]pyrazine

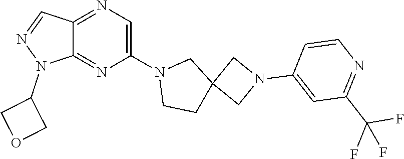 [SMILES: FC(F)(F)c1cc(N2CC3(CCN(c4cnc5cnn(C6COC6)c5n4)C3)C2)ccn1]

Step 1: 1-(oxetan-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure I using 2-(2-(trifluoromethyl)pyridin-4-yl)-2,6-diazaspiro[3.4]octane hydrochloride (3.84 g, 13.1 mmol, 1.1 equiv.) and 6-chloro-1-(oxetan-3-yl)-1H-pyrazolo[3,4-b]pyrazine (2.5 g, 11.8 mmol, 1.0 equiv.) as the starting materials to give 1-(oxetan-3-yl)-6-(2-(2-(trifluoromethyl)pyridin-4-yl)-2,6-diazaspiro[3.4]octan-6-yl)-1H-pyrazolo[3,4-b]pyrazine (3.45 g, 67%) as a white solid. ¹H NMR (400 MHz, DMSO-d₆) δ 8.25 (d, J=5.6 Hz, 1H), 8.19 (s, 1H), 8.09 (s, 1H), 6.76 (d, J=2.2 Hz, 1H), 6.58 (dd, J=5.7, 2.2 Hz, 1H), 5.94-5.82 (m, 1H), 5.12-5.02 (m, 2H), 5.02-4.92 (m, 2H), 4.04 (q, J=8.4 Hz, 4H), 3.86 (s, 2H), 3.66 (t, J=6.8 Hz, 2H), 2.31 (t, J=7.0 Hz, 2H). MS m/z: 432.15 [M+H]⁺.

### Example 988: 1-(2,2-difluoroethyl)-5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one

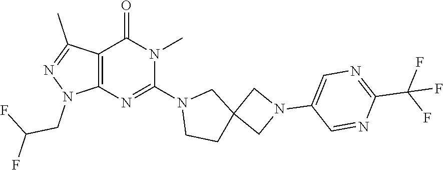 [SMILES: Cc1nn(CC(F)F)c2nc(N3CCC4(CN(c5cnc(C(F)(F)F)nc5)C4)C3)n(C)c(=O)c12]

Step 1: tert-butyl 2-(2-(trifluoromethyl)pyrimidin-5-yl)-2,6-diazaspiro[3.4]octane-6-carboxylate: Followed the general procedure Y using tert-butyl 2,6-diazaspiro[3.4]octane-6-carboxylate (200 mg, 0.943 mmol, 1.0 equiv.) and 5-bromo-2-(trifluoromethyl)pyrimidine (214 mg, 0.943 mmol, 1.0 equiv.) as the starting materials to give tert-butyl 2-(2-(trifluoromethyl)pyrimidin-5-yl)-2,6-diazaspiro[3.4]octane-6-carboxylate (200 mg, 80%) as a white solid. MS m/z: 359 [M+H]⁺.

Step 2: 2-(2-(trifluoromethyl)pyrimidin-5-yl)-2,6-diazaspiro[3.4]octane: Followed the general procedure H using tert-butyl 2-(2-(trifluoromethyl)pyrimidin-5-yl)-2,6-diazaspiro[3.4]octane-6-carboxylate (100 mg) as the starting material to give the crude product 2-(2-(trifluoromethyl)pyrimidin-5-yl)-2,6-diazaspiro[3.4]octane (100 mg). MS m/z: 259 [M+H]⁺.

Step 3: 1-(2,2-difluoroethyl)-5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one: Followed the general procedure I using 2-(2-(trifluoromethyl)pyrimidin-5-yl)-2,6-diazaspiro[3.4]octane (100 mg, 0.386 mmol, 1.0 equive.) and 6-chloro-1-(2,2-difluoroethyl)-5-methyl-1,5-dihydro-4H-pyrazolo[3,4-d]pyrimidin-4-one (96.1 mg, 0.386 mmol, 1.0 equiv.) as the starting materials to give 1-(2,2-difluoroethyl)-5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (40 mg, 21%) as a white solid. ¹H NMR (300 MHz, DMSO-d₆) δ 8.17 (s, 2H), 7.96 (s, 1H), 6.42 (tt, 1H), 4.58 (td, 2H), 4.08 (q, 4H), 3.83 (s, 2H), 3.66 (t, 2H), 3.40 (s, 3H), 2.23 (t, 2H). MS m/z: 471.15 [M+H]⁺.

### Example 989: 1-(2,2-difluoroethyl)-5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one

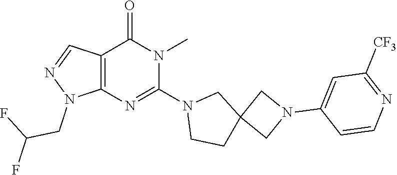 [SMILES: Cn1c(N2CCC3(CN(c4ccnc(C(F)(F)F)c4)C3)C2)nc2c(cnn2CC(F)F)c1=O]

Step 1: methyl 6-(trifluoromethoxy)nicotinate: Followed the general procedure I using 2-(2-(trifluoromethyl)pyridin-4-yl)-2,6-diazaspiro[3.4]octane (70 mg, 272 μmol, 1.0 equiv.) and 6-chloro-1-(2,2-difluoroethyl)-5-methyl-1,5-dihydro-4H-pyrazolo[3,4-d]pyrimidin-4-one (68 mg, 272 μmol, 1.0 equiv.) as the starting materials to give 1-(2,2-difluoroethyl)-5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (45.1 mg, 45%) as a white solid. ¹H NMR (300 MHz, DMSO-d₆) δ 8.25 (d, 1H), 7.96 (s, 1H), 6.75 (d, 1H), 6.62-6.23 (m, 2H), 4.59 (m, 2H), 4.02 (q, 4H), 3.82 (s, 2H), 3.65 (t, 2H), 3.40 (s, 3H), 2.21 (t, 2H). MS m/z: 470.15 [M+H]⁺.

### Example 990: 1-ethyl-5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one

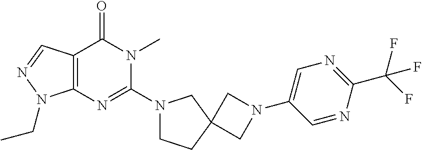 [SMILES: CCn1ncc2c(=O)n(C)c(N3CCC4(CN(c5cnc(C(F)(F)F)nc5)C4)C3)nc21]

Step 1: 2-(2-(trifluoromethyl)pyrimidin-5-yl)-2,6-diazaspiro[3.4]octane: Followed the general procedure H using tert-butyl 2-(2-(trifluoromethyl)pyrimidin-5-yl)-2,6-diazaspiro[3.4]octane-6-carboxylate (120 mg, 0.335 mmol, 1.0 equiv.) as the starting material to give the crude product 2-(2-(trifluoromethyl)pyrimidin-5-yl)-2,6-diazaspiro[3.4]octane (80 mg). MS m/z: 259 [M+H]⁺

Step 2: 5-methyl-1-(tetrahydro-2H-pyran-2-yl)-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one: Followed the general procedure I using 2-(2-(trifluoromethyl)pyrimidin-5-yl)-2,6-diazaspiro[3.4]octane (80 mg, 0.31 mmol, 1.0 equiv.) and 6-chloro-5-methyl-1-(tetrahydro-2H-pyran-2-yl)-1,5-dihydro-4H-pyrazolo[3,4-d]pyrimidin-4-one (83 mg, 0.31 mmol, 1.0 equiv.) as the starting materials to give 5-methyl-1-(tetrahydro-2H-pyran-2-yl)-6-(2-(2-(trifluoromethyl) pyrimidin-5-yl)-2,6-diazaspiro[3.4]octan-6-yl)-1,5-dihydro-4H-pyrazolo[3,4-d]pyrimidin-4-one (70 mg, 46%) as a yellow solid. MS m/z: 491 [M+H]⁺

Step 3: 5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one: Followed the general procedure H using 5-methyl-1-(tetrahydro-2H-pyran-2-yl)-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (70 mg, 0.142 mmol, 1.0 equiv.) as the starting material to give the crude product 5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (60 mg). MS m/z: 407 [M+H]⁺

Step 4: 1-ethyl-5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one: Followed the general procedure K using 5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (40 mg, 0.098 mmol, 1.0 equiv.) and iodoethane (15.3 mg, 0.098 mmol, 1.0 equiv.) as the starting materials to give 1-ethyl-5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (10.4 mg, 24%) as a white solid. ¹H NMR (400 MHz, DMSO-d₆) δ 8.17 (s, 2H), 7.85 (d, J=1.1 Hz, 1H), 4.16 (q, J=7.2 Hz, 2H), 4.07 (q, J=8.2 Hz, 4H), 3.79 (s, 2H), 3.62 (t, J=6.8 Hz, 2H), 3.38 (s, 3H), 2.21 (t, J=6.9 Hz, 2H), 1.37-1.33 (m, 3H). MS m/z: 435.05 [M+H]⁺.

### Example 991: 1-(2,2-difluoroethyl)-5-methyl-6-(2-(2-(trifluoromethyl)pyrimidin-4-yl)-2,6-diazaspiro[3.4]octan-6-yl)-1,5-dihydro-4H-pyrazolo[3,4-d]pyrimidin-4-one

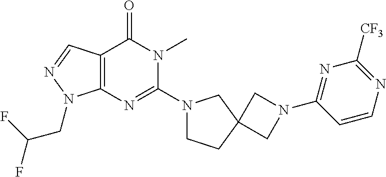 [SMILES: Cn1c(N2CCC3(CN(c4ccnc(C(F)(F)F)n4)C3)C2)nc2c(cnn2CC(F)F)c1=O]

Step 1: tert-butyl 2-(2-(trifluoromethyl)pyrimidin-4-yl)-2,6-diazaspiro[3.4]octane-6-carboxylate: Followed the general procedure I using tert-butyl 2,6-diazaspiro[3.4]octane-6-carboxylate (100 mg, 471 μmol, 1.0 equiv.) and 4-chloro-2-(trifluoromethyl)pyrimidine (86 mg, 471 μmol, 1.0 equiv.) as the starting materials to give tert-butyl 2-(2-(trifluoromethyl)pyrimidin-4-yl)-2,6-diazaspiro[3.4]octane-6-carboxylate (110 mg, 65%) as a white solid. MS m/z: 359 [M+H]⁺.

Step 2: 2-(2-(trifluoromethyl)pyrimidin-4-yl)-2,6-diazaspiro[3.4]octane: Followed the general procedure H using tert-butyl 2-(2-(trifluoromethyl)pyrimidin-4-yl)-2,6-diazaspiro[3.4]octane-6-carboxylate (100 mg) as the starting material to give the crude product 2-(2-(trifluoromethyl)pyrimidin-4-yl)-2,6-diazaspiro[3.4]octane (70 mg). MS m/z: 259 [M+H]⁺.

Step 3: 1-(2,2-difluoroethyl)-5-methyl-6-(2-(2-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure I using 2-(2-(trifluoromethyl)pyrimidin-4-yl)-2,6-diazaspiro[3.4]octane (65 mg, 252 μmol, 1.0 equiv.) and 6-chloro-1-(2,2-difluoroethyl)-5-methyl-1,5-dihydro-4H-pyrazolo[3,4-d]pyrimidin-4-one (62.6 mg, 252 μmol, 1.0 equiv.) as the starting materials to give 1-(2,2-difluoroethyl)-5-methyl-6-(2-(2-(trifluoromethyl)pyrimidin-4-yl)-2,6-diazaspiro[3.4]octan-6-yl)-1,5-dihydro-4H-pyrazolo[3,4-d]pyrimidin-4-one (88 mg, 74%) as a white solid. ¹H NMR (400 MHz, DMSO-d₆) δ 8.31 (d, J=6.0 Hz, 1H), 7.95 (s, 1H), 6.62 (d, J=6.0 Hz, 1H), 6.58-6.26 (m, 1H), 4.58 (td, J=14.8, 4.0 Hz, 2H), 4.19-4.02 (m, 4H), 3.82 (s, 2H), 3.65 (t, J=6.9 Hz, 2H), 3.39 (s, 3H), 2.20 (t, J=6.8 Hz, 2H). MS m/z: 471.15 [M+H]⁺.

### Example 992: 1-(2,2-difluoroethyl)-5-methyl-6-(5-oxo-2-(2-(trifluoromethyl)pyridin-4-yl)-2,6-diazaspiro[3.4]octan-6-yl)-1,5-dihydro-4H-pyrazolo[3,4-d]pyrimidin-4-one

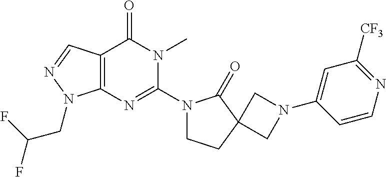 [SMILES: Cn1c(N2CCC3(CN(c4ccnc(C(F)(F)F)c4)C3)C2=O)nc2c(cnn2CC(F)F)c1=O]

Step 1: 1-(2,2-difluoroethyl)-5-methyl-6-(5-oxo-2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure Y using 2-(2-(trifluoromethyl)pyridin-4-yl)-2,6-diazaspiro[3.4]octan-5-one (80 mg, 0.295 mmol, 1.0 equiv.) and 6-chloro-1-(2,2-difluoroethyl)-5-methylpyrazolo[3,4-d]pyrimidin-4-one (88.0 mg, 0.354 mmol, 1.2 equiv.) as the starting materials, EPhos Pd G4 (27.1 mg, 0.029 mmol, 0.1 equiv.) as the catalyst to give 1-(2,2-difluoroethyl)-5-methyl-6-(5-oxo-2-(2-(trifluoromethyl)pyridin-4-yl)-2,6-diazaspiro[3.4]octan-6-yl)-1,5-dihydro-4H-pyrazolo[3,4-d]pyrimidin-4-one (11 mg, 7%) as a white solid. ¹H NMR (400 MHz, DMSO-d₆) δ 8.31-8.21 (m, 2H), 6.82 (d, J=2.3 Hz, 1H), 6.64-6.32 (m, 2H), 4.72 (td, J=15.0, 3.7 Hz, 2H), 4.20 (d, J=8.5 Hz, 2H), 4.12 (d, J=8.5 Hz, 2H), 3.97 (s, 2H), 3.42 (s, 3H), 2.63 (t, J=6.9 Hz, 2H). MS m/z: 484.00 [M+H]⁺.

### Example 993: 5-methyl-1-(oxetan-3-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one

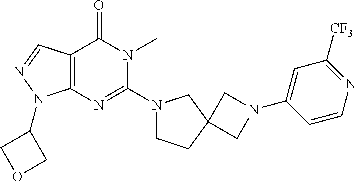 [SMILES: Cn1c(N2CCC3(CN(c4ccnc(C(F)(F)F)c4)C3)C2)nc2c(cnn2C2COC2)c1=O]

Step 1: 5-methyl-1-(oxetan-3-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure K using 5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (80.0 mg, 0.197 mmol, 1.00 equiv) and 3-iodooxetane (182 mg, 0.985 mmol, 5.00 equiv) as the starting materials to give 5-methyl-1-(oxetan-3-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (20.6 mg, 21.6%) as a white solid. ¹H NMR (300 MHz, Chloroform-d) δ 8.34 (d, 1H), 8.04 (s, 1H), 6.63 (s, 1H), 6.39 (d, 1H), 5.85-5.75 (m, 1H), 5.33-5.23 (m, 2H), 5.05-4.99 (m, 2H), 4.10-4.00 (m, 4H), 3.80 (s, 2H), 3.67 (t, 2H), 3.51 (s, 3H), 2.30 (t, 2H). MS m/z: 462.05 [M+H]⁺.

### Example 994: 1-(2,2-difluoroethyl)-3,5-dimethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one

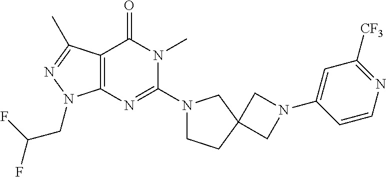 [SMILES: Cc1nn(CC(F)F)c2nc(N3CCC4(CN(c5ccnc(C(F)(F)F)c5)C4)C3)n(C)c(=O)c12]

Step 1: 3-iodo-5-methyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure I using 2-(2-(trifluoromethyl)pyridin-4-yl)-2,6-diazaspiro[3.4]octane hydrochloride (250 mg, 972 μmol, 1.0 equiv.) and 6-chloro-3-iodo-5-methyl-1-(tetrahydro-2H-pyran-2-yl)-1,5-dihydro-4H-pyrazolo[3,4-d]pyrimidin-4-one (460 mg, 1.16 mmol, 1.2 equiv.) as the starting materials to give 3-iodo-5-methyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (500 mg, 84%) as a white solid. MS m/z: 616 [M+H]⁺.

Step 2: 3,5-dimethyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure L using 3-iodo-5-methyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (300 mg, 487 μmol, 1.0 equiv.) and methylboronic acid (58.4 mg, 974 μmol, 2.0 equiv.) as the starting materials to give 3,5-dimethyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (160 mg, 65%) as a white solid. MS m/z: 504 [M+H]⁺.

Step 3: 3,5-dimethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure M using 3,5-dimethyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (150 mg, 298 mmol, 1.0 equiv.) as the starting material to give 3,5-dimethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (70 mg, 56%) as a white solid. MS m/z: 420 [M+H]⁺.

Step 4: 1-(2,2-difluoroethyl)-3,5-dimethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure K using 3,5-dimethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (70 mg, 167 μmol, 1.0 equiv.) and 2,2-difluoroethyl trifluoromethanesulfonate (71.5 mg, 334 μmol, 2.0 equiv.) as the starting materials to give 1-(2,2-difluoroethyl)-3,5-dimethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (19.1 mg, 23%) as a white solid. ¹H NMR (400 MHz, DMSO-d₆) δ 8.24 (d, J=5.6 Hz, 1H), 6.74 (d, J=2.2 Hz, 1H), 6.61-6.22 (m, 2H), 4.56-4.41 (m, 2H), 4.08-3.95 (m, 4H), 3.80 (s, 2H), 3.63 (t, J=6.8 Hz, 2H), 3.37 (s, 3H), 2.36 (s, 3H), 2.19 (t, J=6.8 Hz, 2H). MS m/z: 484.1 [M+H]⁺.

### Example 995: 3,5-dimethyl-1-(oxetan-3-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one

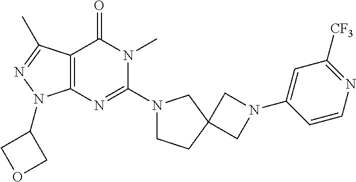 [SMILES: Cc1nn(C2COC2)c2nc(N3CCC4(CN(c5ccnc(C(F)(F)F)c5)C4)C3)n(C)c(=O)c12]

Step 1: 3,5-dimethyl-1-(oxetan-3-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure K using 3,5-dimethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (70 mg, 167 μmol, 1.0 equiv.) and oxetan-3-yl trifluoromethanesulfonate (68.8 mg, 334 μmol, 2.0 equiv.) as the starting materials to give 3,5-dimethyl-1-(oxetan-3-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (18.3 mg, 23%) as a white solid. ¹H NMR (400 MHz, DMSO-d₆) δ 8.24 (d, J=5.6 Hz, 1H), 6.74 (d, J=2.2 Hz, 1H), 6.57 (dd, J=5.7, 2.3 Hz, 1H), 5.75-5.58 (m, 1H), 4.97 (t, J=6.4 Hz, 2H), 4.90 (dd, J=7.8, 6.3 Hz, 2H), 4.10-3.92 (m, 4H), 3.78 (s, 2H), 3.61 (t, J=6.8 Hz, 2H), 3.35 (s, 3H), 2.41 (s, 3H), 2.19 (t, J=6.8 Hz, 2H). MS m/z: 476.19 [M+H]⁺.

### Example 996: 1-(2,2-difluoroethyl)-5-methyl-6-(2-(5-(trifluoromethyl)pyridin-2-yl)-2,6-diazaspiro[3.4]octan-6-yl)-1,5-dihydro-4H-pyrazolo[3,4-d]pyrimidin-4-one

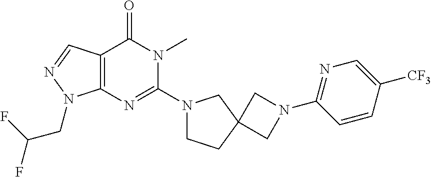 [SMILES: Cn1c(N2CCC3(CN(c4ccc(C(F)(F)F)cn4)C3)C2)nc2c(cnn2CC(F)F)c1=O]

Step 1: tert-butyl 2-(5-(trifluoromethyl)pyridin-2-yl)-2,6-diazaspiro[3.4]octane-6-carboxylate: Followed the general procedure Y using tert-butyl 2,6-diazaspiro[3.4]octane-6-carboxylate (200 mg, 942 μmol, 1.0 equiv.) and 2-bromo-5-(trifluoromethyl)pyridine (425 mg, 1.88 mmol, 2.0 equiv.) as the starting materials to give tert-butyl 2-(5-(trifluoromethyl)pyridin-2-yl)-2,6-diazaspiro[3.4]octane-6-carboxylate (330 mg, 98%) as a white solid. MS m/z: 358 [M+H]⁺.

Step 2: 2-(5-(trifluoromethyl)pyridin-2-yl)-2,6-diazaspiro[3.4]octane: Followed the general procedure H using tert-butyl 2-(5-(trifluoromethyl)pyridin-2-yl)-2,6-diazaspiro[3.4]octane-6-carboxylate (104 mg) as the starting material to give the crude product 2-(5-(trifluoromethyl)pyridin-2-yl)-2,6-diazaspiro[3.4]octane (75 mg). MS m/z: 258 [M+H]⁺.

Step 3: 1-(2,2-difluoroethyl)-5-methyl-6-(2-(5-(trifluoromethyl)pyridin-2-yl)-2,6-diazaspiro[3.4]octan-6-yl)-1,5-dihydro-4H-pyrazolo[3,4-d]pyrimidin-4-one: Followed the general procedure I using 2-(5-(trifluoromethyl)pyridin-2-yl)-2,6-diazaspiro[3.4]octane (65 mg, 253 μmol, 1.0 equiv.) and 6-chloro-1-(2,2-difluoroethyl)-5-methyl-1,5-dihydro-4H-pyrazolo[3,4-d]pyrimidin-4-one (62.8 mg, 253 μmol, 1.0 equiv.) as the starting materials to give 1-(2,2-difluoroethyl)-5-methyl-6-(2-(5-(trifluoromethyl)pyridin-2-yl)-2,6-diazaspiro[3.4]octan-6-yl)-1,5-dihydro-4H-pyrazolo[3,4-d]pyrimidin-4-one (99.6 mg, 82%) as a white solid. ¹H NMR (400 MHz, DMSO-d₆) δ 8.38 (d, J=2.4 Hz, 1H), 7.95 (s, 1H), 7.78 (dd, J=9.0, 2.5 Hz, 1H), 6.63-6.20 (m, 2H), 4.72-4.46 (m, 2H), 4.10-3.98 (m, 4H), 3.82 (s, 2H), 3.66 (t, J=6.8 Hz, 2H), 3.40 (s, 3H), 2.20 (t, J=6.8 Hz, 2H). MS m/z: 470.15 [M+H]⁺.

### Example 997: 1-(2,2-difluoroethyl)-5-methyl-6-(2-(6-(trifluoromethyl)pyridin-3-yl)-2,6-diazaspiro[3.4]octan-6-yl)-1,5-dihydro-4H-pyrazolo[3,4-d]pyrimidin-4-one

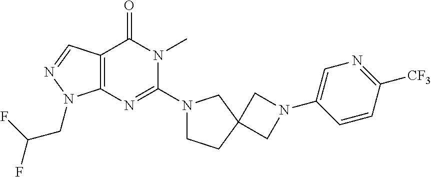 [SMILES: Cn1c(N2CCC3(CN(c4ccc(C(F)(F)F)nc4)C3)C2)nc2c(cnn2CC(F)F)c1=O]

Step 1. tert-butyl 2-(6-(trifluoromethyl)pyridin-3-yl)-2,6-diazaspiro[3.4]octane-6-carboxylate: Followed the general procedure Y using tert-butyl 2,6-diazaspiro[3.4]octane-6-carboxylate (100 mg, 0.472 mmol, 1.0 equiv.) and 5-bromo-2-(trifluoromethyl)pyridine (107 mg, 0.473 mmol, 1.0 equiv.) as the starting materials to give tert-butyl 2-(6-(trifluoromethyl)pyridin-3-yl)-2,6-diazaspiro[3.4]octane-6-carboxylate (200 mg, 90%) as a white solid. MS m/z: 358 [M+H]⁺.

Step 2. 2-(6-(trifluoromethyl)pyridin-3-yl)-2,6-diazaspiro[3.4]octane: Followed the general procedure H using tert-butyl 2-(6-(trifluoromethyl)pyridin-3-yl)-2,6-diazaspiro[3.4]octane-6-carboxylate (200 mg) as the starting material to give the crude product 2-(6-(trifluoromethyl)pyridin-3-yl)-2,6-diazaspiro[3.4]octane (200 mg). MS m/z: 258 [M+H]⁺.

Step 3. 1-(2,2-difluoroethyl)-5-methyl-6-(2-(6-(trifluoromethyl)pyridin-3-yl)-2,6-diazaspiro[3.4]octan-6-yl)-1,5-dihydro-4H-pyrazolo[3,4-d]pyrimidin-4-one: Followed the general procedure I using 2-(6-(trifluoromethyl)pyridin-3-yl)-2,6-diazaspiro[3.4]octane (100 mg, 0.388 μmol, 1.0 equiv.) and 6-chloro-1-(2,2-difluoroethyl)-5-methyl-1,5-dihydro-4H-pyrazolo[3,4-d]pyrimidin-4-on (96.5 mg, 0.388 mmol, 1.0 equiv.) as the starting materials to give 1-(2,2-difluoroethyl)-5-methyl-6-(2-(6-(trifluoromethyl)pyridin-3-yl)-2,6-diazaspiro[3.4]octan-6-yl)-1,5-dihydro-4H-pyrazolo[3,4-d]pyrimidin-4-one (30 mg, 36%) as a white solid. ¹H NMR (400 MHz, DMSO-d₆) δ 8.03-7.85 (m, 2H), 7.61 (d, 1H), 6.99-6.89 (m, 1H), 6.08 (tt, 1H), 4.57 (td, 2H), 4.00 (q, 4H), 3.82 (s, 2H), 3.65 (t, 2H), 3.40 (s, 3H), 2.21 (t, 2H). MS m/z: 470.15 [M+H]⁺.

### Example 998: 8-(1-(2,2-difluoroethyl)-1H-pyrazolo[3,4-b]pyrazin-6-yl)-2-((6-(trifluoromethoxy)pyridin-3-yl)methyl)-2,8-diazaspiro[4.5]decan-3-one

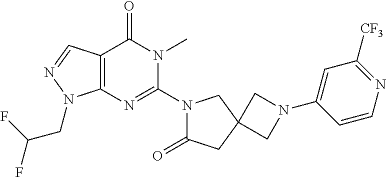 [SMILES: Cn1c(N2CC3(CC2=O)CN(c2ccnc(C(F)(F)F)c2)C3)nc2c(cnn2CC(F)F)c1=O]

Step 1: 2-(2-(trifluoromethyl)pyridin-4-yl)-2,6-diazaspiro[3.4]octan-7-one: Followed the general procedure I using 2,6-diazaspiro[3.4]octan-7-one (500 mg, 3.97 mmol, 1.0 equiv.) and 4-chloro-2-(trifluoromethyl)pyridine (722 mg, 4.01 mmol, 1.0 equiv.) as the starting materials to give 2-(2-(trifluoromethyl)pyridin-4-yl)-2,6-diazaspiro[3.4]octan-7-one (450 mg, 60%) as a white solid. MS m/z: 272 [M+H]⁺.

Step 2: 1-(2,2-difluoroethyl)-5-methyl-6-(7-oxo-2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure Y using 2-(2-(trifluoromethyl)pyridin-4-yl)-2,6-diazaspiro[3.4]octan-7-one (70.0 mg, 0.257 mmol, 1.0 equiv.) and 6-chloro-1-(2,2-difluoroethyl)-5-methyl-1,5-dihydro-4H-pyrazolo[3,4-d]pyrimidin-4-one (64.1 mg, 0.257 mmol, 1.0 equiv.) as the starting materials, EPhos Pd G4 (2.37 mg, 0.03 mmol, 0.1 equiv.) as the catalyst to give 1-(2,2-difluoroethyl)-5-methyl-6-(7-oxo-2-(2-(trifluoromethyl)pyridin-4-yl)-2,6-diazaspiro[3.4]octan-6-yl)-1,5-dihydro-4H-pyrazolo[3,4-d]pyrimidin-4-one (47.4 mg, 39%) as a white solid. ¹H NMR (400 MHz, DMSO-d₆) δ 8.26 (d, 1H), 8.22 (s, 1H), 6.78 (d, Hz, 1H), 6.64-6.28 (m, 2H), 4.71 (td, 2H), 4.25 (s, 2H), 4.14 (q, Hz, 4H), 3.41 (s, 3H), 3.02 (s, 2H). MS m/z: 484.1 [M+H]⁺.

### Example 999: 6-(1-(2,2-difluoroethyl)-1H-pyrazolo[3,4-d]pyrimidin-6-yl)-2-(2-(trifluoromethyl)pyridin-4-yl)-2,6-diazaspiro[3.4]octan-5-one

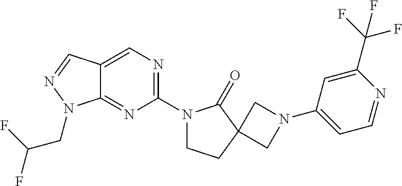 [SMILES: O=C1N(c2ncc3cnn(CC(F)F)c3n2)CCC12CN(c1ccnc(C(F)(F)F)c1)C2]

Step 1. 6-(1-(2,2-difluoroethyl)-1H-pyrazolo[3,4-d]pyrimidin-6-yl)-2-(2-(trifluoromethyl)pyridin-4-yl)-2,6-diazaspiro[3.4]octan-5-one: Followed the general procedure Y using 2-(2-(trifluoromethyl)pyridin-4-yl)-2,6-diazaspiro[3.4]octan-5-one (80 mg, 0.295 mmol, 1.0 equiv.) and 6-chloro-1-(2,2-difluoroethyl)-1H-pyrazolo[3,4-d]pyrimidine (71 mg, 0.325 mmol, 1.1 equiv.) as the starting materials, EPhos Pd G3 (18.4 mg, 0.071 mmol, 0.1 equiv.), EPhos (12.6 mg, 0.076 mmol, 0.2 equiv.) as the catalysts to give 6-(1-(2,2-difluoroethyl)-1H-pyrazolo[3,4-d]pyrimidin-6-yl)-2-(2-(trifluoromethyl)pyridin-4-yl)-2,6-diazaspiro[3.4]octan-5-one (12.5 mg, 15%) as white solid. ¹H NMR (400 MHz, DMSO-d₆) δ 9.31 (s, 1H), 8.41 (s, 1H), 8.28 (d, J=5.7 Hz, 1H), 6.81 (d, J=2.2 Hz, 1H), 6.76-6.56 (m, 1H), 6.54 (s, 1H), 5.03-4.63 (m, 2H), 4.21 (d, J=8.4 Hz, 2H), 4.17-3.37 (m, 4H), 2.58-2.52 (m, 2H). MS m/z: 454.05 [M+H]⁺.

### Example 1000: 6-(1-(2,2-difluoroethyl)-3-methyl-1H-pyrazolo[3,4-d]pyrimidin-6-yl)-2-(2-(trifluoromethyl)pyridin-4-yl)-2,6-diazaspiro[3.4]octan-5-one

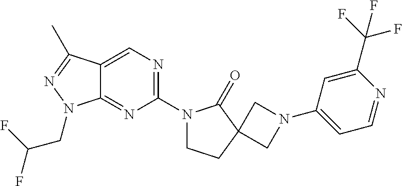 [SMILES: Cc1nn(CC(F)F)c2nc(N3CCC4(CN(c5ccnc(C(F)(F)F)c5)C4)C3=O)ncc12]

Step 1. 6-(1-(2,2-difluoroethyl)-3-methyl-1H-pyrazolo[3,4-d]pyrimidin-6-yl)-2-(2-(trifluoromethyl)pyridin-4-yl)-2,6-diazaspiro[3.4]octan-5-one: Followed the general procedure Y using 2-(2-(trifluoromethyl)pyridin-4-yl)-2,6-diazaspiro[3.4]octan-5-one (80 mg, 0.295 mmol, 1.0 equiv.) and 6-chloro-1-(2,2-difluoroethyl)-3-methyl-1H-pyrazolo[3,4-d]pyrimidine (71 mg, 0.325 mmol, 1.1 equiv.) as the starting materials, EPhos Pd G3 (20.4 mg, 0.03 mmol, 0.1 equiv.), EPhos (14.2 mg, 0.06 mmol, 0.2 equiv.) as the catalysts to give 6-(1-(2,2-difluoroethyl)-3-methyl-1H-pyrazolo[3,4-d]pyrimidin-6-yl)-2-(2-(trifluoromethyl)pyridin-4-yl)-2,6-diazaspiro[3.4]octan-5-one (13.4 mg, 15%) as a white solid. ¹H NMR (400 MHz, DMSO-d₆) δ 9.48-9.15 (m, 1H), 8.28 (d, J=5.5 Hz, 1H), 6.81 (d, J=2.6 Hz, 1H), 6.66-6.26 (m, 2H), 4.74 (dd, J=16.6, 13.3 Hz, 2H), 4.21 (d, J=8.6 Hz, 2H), 4.11-4.01 (m, 4H), 2.57-2.55 (m, 3H), 2.53 (s, 2H). MS m/z: 468.1 [M+H]⁺.

### Example 1001: 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-5-one

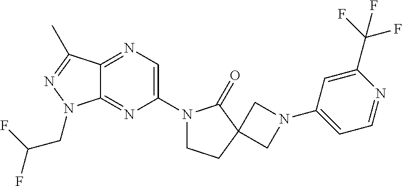 [SMILES: Cc1nn(CC(F)F)c2nc(N3CCC4(CN(c5ccnc(C(F)(F)F)c5)C4)C3=O)cnc12]

Step 1: tert-butyl 6-(1-(2,2-difluoroethyl)-3-methyl-1H-pyrazolo[3,4-b]pyrazin-6-yl)-5-oxo-2,6-diazaspiro[3.4]octane-2-carboxylate: Followed the general procedure Y using 6-chloro-1-(2,2-difluoroethyl)-3-methyl-1H-pyrazolo[3,4-b]pyrazine (70 mg, 0.301 mmol, 1.0 equiv.) and tert-butyl 5-oxo-2,6-diazaspiro[3.4]octane-2-carboxylate (82 mg, 0.361 mmol, 1.2 equiv.) as the starting materials, EPhos (33.3 mg, 0.062 mmol, 0.2 equiv.), EPhos Pd G4 (28.6 mg, 0.031 mmol, 0.1 equiv.) to give tert-butyl 6-(1-(2,2-difluoroethyl)-3-methyl-1H-pyrazolo[3,4-b]pyrazin-6-yl)-5-oxo-2,6-diazaspiro[3.4]octane-2-carboxylate (50 mg, 80%) as white solid. MS m/z: 423 [M+H]⁺.

Step 2. 6-(1-(2,2-difluoroethyl)-3-methyl-1H-pyrazolo[3,4-b]pyrazin-6-yl)-2,6-diazaspiro[3.4]octan-5-one: Followed the general procedure H using tert-butyl 6-(1-(2,2-difluoroethyl)-3-methyl-1H-pyrazolo[3,4-b]pyrazin-6-yl)-5-oxo-2,6-diazaspiro[3.4]octane-2-carboxylate (50 mg) as the starting material to give the crude product 6-(1-(2,2-difluoroethyl)-3-methyl-1H-pyrazolo[3,4-b]pyrazin-6-yl)-2,6-diazaspiro[3.4]octan-5-one (40 mg) as a brow oil. MS m/z: 323 [M+H]⁺.

Step 3. 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-5-one: Followed the general procedure I using 6-(1-(2,2-difluoroethyl)-3-methyl-1H-pyrazolo[3,4-b]pyrazin-6-yl)-2,6-diazaspiro[3.4]octan-5-one (40 mg, 0.124 mmol, 1.0 equiv.) and 4-chloro-2-(trifluoromethyl)pyridine (27 mg, 0.149 mmol, 1.2 equiv.) as the starting materials to give 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-5-one (13.6 mg, 30%) as a white solid. ¹H NMR (400 MHz, DMSO-d₆) δ 9.64 (s, 1H), 8.28 (d, J=5.6 Hz, 1H), 6.82 (d, J=2.2 Hz, 1H), 6.67-6.59 (m, 1H), 6.50-6.33 (m, 1H), 4.86-4.73 (m, 2H), 4.25 (d, J=8.5 Hz, 2H), 4.14-4.02 (m, 4H), 2.59 (d, J=6.9 Hz, 3H), 2.55 (s, 2H). MS m/z: 468.05[M+H]⁺.

### Example 1002: 6-(3-methyl-1-(oxetan-3-yl)-1H-pyrazolo[3,4-b]pyrazin-6-yl)-2-(2-(trifluoromethyl)pyridin-4-yl)-2,6-diazaspiro[3.4]octan-5-one

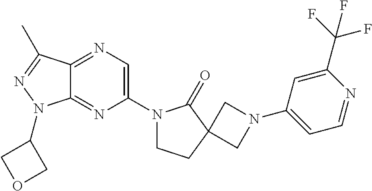 [SMILES: Cc1nn(C2COC2)c2nc(N3CCC4(CN(c5ccnc(C(F)(F)F)c5)C4)C3=O)cnc12]

Step 1: tert-butyl 6-(3-methyl-1-(oxetan-3-yl)-1H-pyrazolo[3,4-b]pyrazin-6-yl)-5-oxo-2,6-diazaspiro[3.4]octane-2-carboxylate: Followed the general procedure Y using 6-chloro-3-methyl-1-(oxetan-3-yl)-1H-pyrazolo[3,4-b]pyrazine (70 mg, 0.312 mmol, 1.0 equiv.) and tert-butyl 5-oxo-2,6-diazaspiro[3.4]octane-2-carboxylate (71 mg, 0.312 mmol, 1.0 equiv.) as the starting materials, EPhos (33.3 mg, 0.062 mmol, 0.2 equiv.), EPhos Pd G4 (28.6 mg, 0.031 mmol, 0.1 equiv.) as the catalysts to give tert-butyl 6-(3-methyl-1-(oxetan-3-yl)-1H-pyrazolo[3,4-b]pyrazin-6-yl)-5-oxo-2,6-diazaspiro[3.4]octane-2-carboxylate (60 mg, 28%) as a brown oil. MS m/z: 415 [M+H]⁺.

Step 2: 6-(3-methyl-1-(oxetan-3-yl)-1H-pyrazolo[3,4-b]pyrazin-6-yl)-2,6-diazaspiro[3.4]octan-5-one hydrochloride: Followed the general procedure B using tert-butyl 6-(3-methyl-1-(oxetan-3-yl)-1H-pyrazolo[3,4-b]pyrazin-6-yl)-5-oxo-2,6-diazaspiro[3.4]octane-2-carboxylate (60 mg) as the starting material to give the crude product 6-(3-methyl-1-(oxetan-3-yl)-1H-pyrazolo[3,4-b]pyrazin-6-yl)-2,6-diazaspiro[3.4]octan-5-one hydrochloride (40 mg). MS m/z: 315 [M+H]⁺.

Step 3: 6-(3-methyl-1-(oxetan-3-yl)-1H-pyrazolo[3,4-b]pyrazin-6-yl)-2-(2-(trifluoromethyl)pyridin-4-yl)-2,6-diazaspiro[3.4]octan-5-one: Followed the general procedure I using 6-(3-methyl-1-(oxetan-3-yl)-1H-pyrazolo[3,4-b]pyrazin-6-yl)-2,6-diazaspiro[3.4]octan-5-one hydrochloride (40 mg, 0.127 mmol, 1.0 equiv.) and 4-bromo-2-(trifluoromethyl)pyridine (27.7 mg, 0.152 mmol, 1.2 equiv.) as the starting materials to give 6-(3-methyl-1-(oxetan-3-yl)-1H-pyrazolo[3,4-b]pyrazin-6-yl)-2-(2-(trifluoromethyl)pyridin-4-yl)-2,6-diazaspiro[3.4]octan-5-one (29.5 mg, 48%) as a white solid. ¹H NMR (400 MHz, DMSO-d₆) δ 9.61 (s, 1H), 8.28 (d, J=5.6 Hz, 1H), 6.82 (d, J=2.2 Hz, 1H), 6.66-6.60 (m, 1H), 6.00-5.91 (m, 1H), 5.09 (t, J=6.4 Hz, 2H), 5.00 (t, J=7.1 Hz, 2H), 4.24 (d, J=8.5 Hz, 2H), 4.18-4.05 (m, 4H), 2.61-2.58 (m, 5H). MS m/z: 460 [M+H]⁺.

### Example 1003: 6-(1-(oxetan-3-yl)-1H-pyrazolo[3,4-b]pyrazin-6-yl)-2-(2-(trifluoromethyl)pyridin-4-yl)-2,6-diazaspiro[3.4]octan-5-one

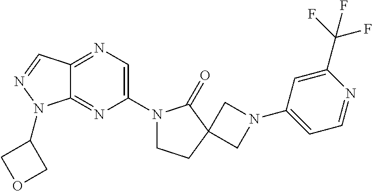 [SMILES: O=C1N(c2cnc3cnn(C4COC4)c3n2)CCC12CN(c1ccnc(C(F)(F)F)c1)C2]

Step 1: 6-(1-(oxetan-3-yl)-1H-pyrazolo[3,4-b]pyrazin-6-yl)-2-(2-(trifluoromethyl)pyridin-4-yl)-2,6-diazaspiro[3.4]octan-5-one: Followed the general procedure Y using 2-(2-(trifluoromethyl)pyridin-4-yl)-2,6-diazaspiro[3.4]octan-5-one (40 mg, 0.147 mmol, 1.0 equiv.) and 6-chloro-1-(oxetan-3-yl)-1H-pyrazolo[3,4-b]pyrazine (37.3 mg, 0.176 mmol, 1.2 equiv) as the starting materials, EPhos Pd G4 (27.1 mg, 0.0290 mmol, 0.2 equiv) as the catalyst to give 6-(1-(oxetan-3-yl)-1H-pyrazolo[3,4-b]pyrazin-6-yl)-2-(2-(trifluoromethyl)pyridin-4-yl)-2,6-diazaspiro[3.4]octan-5-one (25 mg, 57%) as a white solid. ¹H NMR (400 MHz, DMSO-d₆) δ 9.67 (s, 1H), 8.56 (s, 1H), 8.28 (d, J=5.6 Hz, 1H), 6.82 (d, J=2.3 Hz, 1H), 6.63 (dd, J=5.7, 2.3 Hz, 1H), 6.04-5.93 (m, J=6.9 Hz, 1H), 5.11 (t, J=6.4 Hz, 2H), 5.03 (t, J=7.2 Hz, 2H), 4.25 (d, J=8.5 Hz, 2H), 4.08 (dd, J=20.9, 7.8 Hz, 4H), 2.59 (t, J=7.0 Hz, 2H). MS m/z: 446.05 [M+H]⁺.

### Example 1004: 6-(1-(oxetan-3-yl)-1H-pyrazolo[3,4-d]pyrimidin-6-yl)-2-(2-(trifluoromethyl)pyridin-4-yl)-2,6-diazaspiro[3.4]octan-5-one

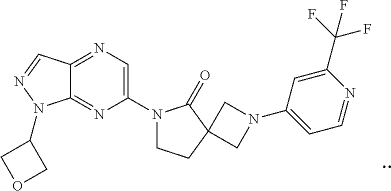 [SMILES: O=C1N(c2cnc3cnn(C4COC4)c3n2)CCC12CN(c1ccnc(C(F)(F)F)c1)C2]

Step 1: 6-chloro-1-(oxetan-3-yl)-1H-pyrazolo[3,4-d]pyrimidine: Followed the general procedure K using 6-chloro-1H-pyrazolo[3,4-d]pyrimidine (500 mg, 3.25 mmol, 1.0 equiv.) and oxetan-3-yl trifluoromethanesulfonate (803 mg, 3.9 mmol, 1.2 equiv.) as the starting materials to give 6-chloro-1-(oxetan-3-yl)-1H-pyrazolo[3,4-d]pyrimidine (210 mg, 31%) as a white solid. MS m/z: 211 [M+H]⁺.

Step 2: tert-butyl 6-(1-(oxetan-3-yl)-1H-pyrazolo[3,4-d]pyrimidin-6-yl)-5-oxo-2,6-diazaspiro[3.4]octane-2-carboxylate: Followed the general procedure Y using 6-chloro-1-(oxetan-3-yl)-1H-pyrazolo[3,4-d]pyrimidine (100 mg, 476 μmol, 1.0 equiv.) and tert-butyl 5-oxo-2,6-diazaspiro[3.4]octane-2-carboxylate (129 mg, 571 μmol, 1.2 equiv.) as the starting materials to give tert-butyl 6-(1-(oxetan-3-yl)-1H-pyrazolo[3,4-d]pyrimidin-6-yl)-5-oxo-2,6-diazaspiro[3.4]octane-2-carboxylate (70 mg, 37%) as a yellow solid. MS m/z: 401 [M+H]⁺.

Step 3: 6-(1-(oxetan-3-yl)-1H-pyrazolo[3,4-d]pyrimidin-6-yl)-2,6-diazaspiro[3.4]octan-5-one: Followed the general procedure H using tert-butyl 6-(1-(oxetan-3-yl)-1H-pyrazolo[3,4-d]pyrimidin-6-yl)-5-oxo-2,6-diazaspiro[3.4]octane-2-carboxylate (70 mg) as the starting material to give the crude product 6-(1-(oxetan-3-yl)-1H-pyrazolo[3,4-d]pyrimidin-6-yl)-2,6-diazaspiro[3.4]octan-5-one (70 mg). MS m/z: 301 [M+H]⁺.

Step 4: 6-(1-(oxetan-3-yl)-1H-pyrazolo[3,4-d]pyrimidin-6-yl)-2-(2-(trifluoromethyl)pyridin-4-yl)-2,6-diazaspiro[3.4]octan-5-one: Followed the general procedure I using 6-(1-(oxetan-3-yl)-1H-pyrazolo[3,4-d]pyrimidin-6-yl)-2,6-diazaspiro[3.4]octan-5-one (70 mg, 233 μmol, 1.0 equiv.) and 4-chloro-2-(trifluoromethyl)pyridine (63 mg, 350 μmol, 1.5 equiv.) as the starting materials to give 6-(1-(oxetan-3-yl)-1H-pyrazolo[3,4-d]pyrimidin-6-yl)-2-(2-(trifluoromethyl)pyridin-4-yl)-2,6-diazaspiro[3.4]octan-5-one (16.1 mg, 16%) as a white solid. ¹H NMR (400 MHz, DMSO-d₆) δ 9.29 (s, 1H), 8.46 (s, 1H), 8.28 (d, J=5.7 Hz, 1H), 6.81 (d, J=2.3 Hz, 1H), 6.62 (dd, J=5.6, 2.3 Hz, 1H), 6.04-5.92 (m, 1H), 5.14-5.04 (m, 2H), 5.04-4.9 (m, 2H), 4.20 (d, J=8.4 Hz, 2H), 4.11-4.01 (m, 4H). 2.53 (s, 2H). MS m/z: 446.15 [M+H]⁺.

### Example 1005: 6-(1-(2,2-difluoroethyl)-1H-pyrazolo[3,4-d]pyrimidin-6-yl)-2-(2-(trifluoromethyl)pyridin-4-yl)-2,6-diazaspiro[3.4]octan-7-one

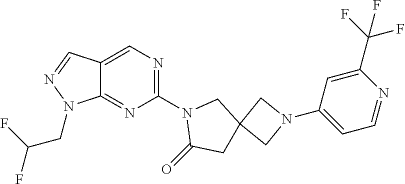 [SMILES: O=C1CC2(CN(c3ccnc(C(F)(F)F)c3)C2)CN1c1ncc2cnn(CC(F)F)c2n1]

Step 1: 6-(1-(2,2-difluoroethyl)-1H-pyrazolo[3,4-d]pyrimidin-6-yl)-2-(2-(trifluoromethyl)pyridin-4-yl)-2,6-diazaspiro[3.4]octan-7-one: Followed the general procedure Y using 2-(2-(trifluoromethyl)pyridin-4-yl)-2,6-diazaspiro[3.4]octan-7-one (70 mg, 0.258 mmol) and 6-chloro-1-(2,2-difluoroethyl)-1H-pyrazolo[3,4-d]pyrimidine (57 mg, 0.258 mmol, 1.0 equiv.) as the starting materials, EPhos Pd G4 (23.7 mg, 0.03 mmol, 0.1 equiv.) to give 6-(1-(2,2-difluoroethyl)-1H-pyrazolo[3,4-d]pyrimidin-6-yl)-2-(2-(trifluoromethyl)pyridin-4-yl)-2,6-diazaspiro[3.4]octan-7-one (34.0 mg, 20%) as a white solid. ¹H NMR (400 MHz, DMSO-d₆) δ 9.29 (s, 1H), 8.40 (s, 1H), 8.26 (d, 1H), 6.77 (d, 1H), 6.67-6.45 (m, 2H), 4.81 (td, 2H), 4.35 (s, 2H), 4.11 (q, 4H), 3.02 (s, 2H). MS m/z: 454.1 [M+H]⁺.

### Example 1006: 6-(1-(2,2-difluoroethyl)-3-methyl-1H-pyrazolo[3,4-d]pyrimidin-6-yl)-2-(2-(trifluoromethyl)pyridin-4-yl)-2,6-diazaspiro[3.4]octan-7-one

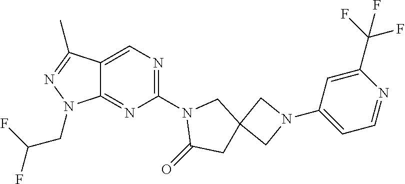 [SMILES: Cc1nn(CC(F)F)c2nc(N3CC4(CC3=O)CN(c3ccnc(C(F)(F)F)c3)C4)ncc12]

Step 1: 6-(1-(2,2-difluoroethyl)-3-methyl-1H-pyrazolo[3,4-d]pyrimidin-6-yl)-2-(2-(trifluoromethyl)pyridin-4-yl)-2,6-diazaspiro[3.4]octan-7-one.

Followed the general procedure Y using 2-(2-(trifluoromethyl)pyridin-4-yl)-2,6-diazaspiro[3.4]octan-7-one (70.0 mg, 0.258 mmol, 1.0 equiv.) and 6-chloro-1-(2,2-difluoroethyl)-3-methyl-1H-pyrazolo[3,4-d]pyrimidine (60.2 mg, 0.258 mmol, 1.0 equiv.) as the starting materials, EPhos Pd G4 (23.7 mg, 0.03 mmol, 0.1 equiv.) as the catalyst to give 6-(1-(2,2-difluoroethyl)-3-methyl-1H-pyrazolo[3,4-d]pyrimidin-6-yl)-2-(2-(trifluoromethyl)pyridin-4-yl)-2,6-diazaspiro[3.4]octan-7-one (30 mg, 25%) as a white solid. ¹H NMR (400 MHz, DMSO-d₆) δ 9.26 (s, 1H), 8.25 (d, 1H), 6.77 (d, 1H), 6.64-6.32 (m, 2H), 4.73 (td, 2H), 4.34 (s, 2H), 4.11 (q, 4H), 3.01 (s, 2H), 2.55 (s, 3H). MS m/z: 468.1 [M+H]⁺.

### Example 1007: 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-7-one

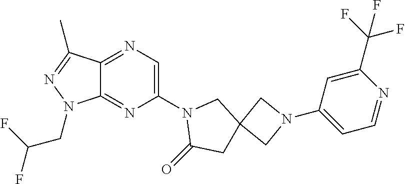 [SMILES: Cc1nn(CC(F)F)c2nc(N3CC4(CC3=O)CN(c3ccnc(C(F)(F)F)c3)C4)cnc12]

Step 1: 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-7-one: Followed the general procedure Y using 2-[2-(trifluoromethyl)pyridin-4-yl]-2,6-diazaspiro[3.4]octan-7-one (80 mg, 0.295 mmol, 1.0 equiv) and 6-chloro-1-(2,2-difluoroethyl)-3-methylpyrazolo[3,4-b]pyrazine (68.6 mg, 0.295 mmol, 1.0 equiv) as the starting materials, EPhos (15.8 mg, 0.03 mmol, 0.1 equiv) and EPhos Pd G4 (27.1 mg, 0.03 mmol, 0.1 equiv) as the catalysts to give 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-7-one (41.3 mg, 30%) as a white solid. ¹H NMR (400 MHz, DMSO-d₆) δ 9.56 (s, 1H), 8.26 (d, 1H), 6.77 (d, 1H), 6.61-6.34 (m, 2H), 4.79 (td, 2H), 4.38 (s, 2H), 4.18-4.10 (m, 4H), 3.12 (s, 2H), 2.55 (s, 3H). MS m/z: 468.1 [M+H]⁺.

### Example 1008: 6-(1-(oxetan-3-yl)-1H-pyrazolo[3,4-d]pyrimidin-6-yl)-2-(2-(trifluoromethyl)pyridin-4-yl)-2,6-diazaspiro[3.4]octan-7-one

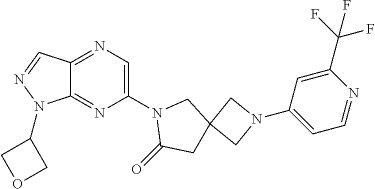 [SMILES: O=C1CC2(CN(c3ccnc(C(F)(F)F)c3)C2)CN1c1cnc2cnn(C3COC3)c2n1]

Step 1: 6-(1-(oxetan-3-yl)-1H-pyrazolo[3,4-d]pyrimidin-6-yl)-2-(2-(trifluoromethyl)pyridin-4-yl)-2,6-diazaspiro[3.4]octan-7-one: Followed the general procedure Y using 2-(2-(trifluoromethyl)pyridin-4-yl)-2,6-diazaspiro[3.4]octan-7-one (80 mg, 0.295 mmol, 1.0 equiv.) and 6-chloro-1-(oxetan-3-yl)-1H-pyrazolo[3,4-d]pyrimidine (62 mg, 0.295 mmol, 1.0 equiv.) as the starting materials, EPhos Pd G4 (27.1 mg, 0.03 mmol, 0.1 equiv.) as the catalyst to give 6-(1-(oxetan-3-yl)-1H-pyrazolo[3,4-d]pyrimidin-6-yl)-2-(2-(trifluoromethyl)pyridin-4-yl)-2,6-diazaspiro[3.4]octan-7-one (28.2 mg, 20%) as a white solid. ¹H NMR (400 MHz, DMSO-d₆) δ 9.27 (s, 1H), 8.45 (s, 1H), 8.26 (d, 1H), 6.78 (d, 1H), 6.59 (dd, 1H), 5.99 (tt, 1H), 5.08 (t, 2H), 5.01 (dd, 2H), 4.34 (s, 2H), 4.11 (q, 4H), 3.02 (s, 2H). MS m/z: 446.2 [M+H]⁺.

### Example 1009: 6-(3-methyl-1-(oxetan-3-yl)-1H-pyrazolo[3,4-d]pyrimidin-6-yl)-2-(2-(trifluoromethyl)pyridin-4-yl)-2,6-diazaspiro[3.4]octan-7-one

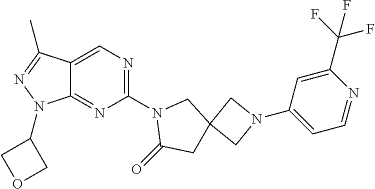 [SMILES: Cc1nn(C2COC2)c2nc(N3CC4(CC3=O)CN(c3ccnc(C(F)(F)F)c3)C4)ncc12]

Step 1: 6-(3-methyl-1-(oxetan-3-yl)-1H-pyrazolo[3,4-d]pyrimidin-6-yl)-2-(2-(trifluoromethyl)pyridin-4-yl)-2,6-diazaspiro[3.4]octan-7-one: Followed the general procedure Y using 2-(2-(trifluoromethyl)pyridin-4-yl)-2,6-diazaspiro[3.4]octan-7-one (70 mg, 0.258 mmol, 1.0 equiv.) and 6-chloro-3-methyl-1-(oxetan-3-yl)-1H-pyrazolo[3,4-d]pyrimidine (59 mg, 0.258 mmol, 1.0 equiv.) as the starting materials, EPhos Pd G4 (23.7 mg, 0.03 mmol, 0.1 equiv.) as the catalyst to give 6-(3-methyl-1-(oxetan-3-yl)-1H-pyrazolo[3,4-d]pyrimidin-6-yl)-2-(2-(trifluoromethyl)pyridin-4-yl)-2,6-diazaspiro[3.4]octan-7-one (10.0 mg, 20%) as a white solid. ¹H NMR (400 MHz, DMSO-d₆) δ 9.24 (s, 1H), 8.26 (d, 1H), 6.77 (d, 1H), 6.59 (dd, 1H), 5.91 (tt, J=7.9, 6.4 Hz, 1H), 5.06 (t, 2H), 4.98 (dd, 2H), 4.33 (s, 2H), 4.10 (q, 4H), 3.01 (s, 2H), 2.60 (s, 3H). MS m/z: 460.15 [M+H]⁺.

### Example 1010: 6-(1-(2,2-difluoroethyl)-1H-pyrazolo[3,4-b]pyrazin-6-yl)-2-(6-(trifluoromethyl)pyridin-3-yl)-2,6-diazaspiro[3.4]octan-5-one

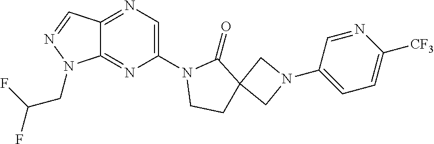 [SMILES: O=C1N(c2cnc3cnn(CC(F)F)c3n2)CCC12CN(c1ccc(C(F)(F)F)nc1)C2]

Step 1: 6-(1-(2,2-difluoroethyl)-1H-pyrazolo[3,4-b]pyrazin-6-yl)-2-(6-(trifluoromethyl)pyridin-3-yl)-2,6-diazaspiro[3.4]octan-5-one: Followed the general procedure I using 6-(1-(2,2-difluoroethyl)-1H-pyrazolo[3,4-b]pyrazin-6-yl)-2,6-diazaspiro[3.4]octan-5-one (80 mg, 258 μmol, 1.0 equiv.) and 5-chloro-2-(trifluoromethyl)pyridine (52 mg, 285 μmol, 1.1 equiv.) as the starting materials to give 6-(1-(2,2-difluoroethyl)-1H-pyrazolo[3,4-b]pyrazin-6-yl)-2-(6-(trifluoromethyl)pyridin-3-yl)-2,6-diazaspiro[3.4]octan-5-one (43 mg, 37%) as a white solid. ¹H NMR (400 MHz, DMSO-d₆) δ 9.70 (s, 1H), 8.51 (s, 1H), 7.98 (d, J=2.8 Hz, 1H), 7.65 (d, J=8.6 Hz, 1H), 7.01 (dd, J=8.4, 2.8 Hz, 1H), 6.55-6.48 (m, 1H), 4.89 (td, J=15.2, 3.6 Hz, 2H), 4.23 (d, J=8.0 Hz, 2H), 4.08 (dd, J=15.1, 7.7 Hz, 4H), 2.60 (t, J=7.0 Hz, 2H). MS m/z: 454.00 [M+H]⁺.

### Example 1011: 6-(1-(2,2-difluoroethyl)-1H-pyrazolo[3,4-b]pyrazin-6-yl)-2-(5-(trifluoromethyl)pyridin-2-yl)-2,6-diazaspiro[3.4]octan-5-one

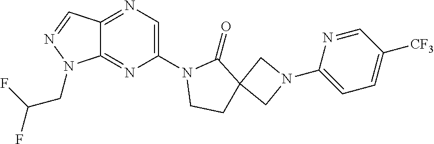 [SMILES: O=C1N(c2cnc3cnn(CC(F)F)c3n2)CCC12CN(c1ccc(C(F)(F)F)cn1)C2]

Step 1: 6-(1-(2,2-difluoroethyl)-1H-pyrazolo[3,4-b]pyrazin-6-yl)-2-(5-(trifluoromethyl)pyridin-2-yl)-2,6-diazaspiro[3.4]octan-5-one: Followed the general procedure I using 6-(1-(2,2-difluoroethyl)-1H-pyrazolo[3,4-b]pyrazin-6-yl)-2,6-diazaspiro[3.4]octan-5-one (80 mg, 258 μmol, 1.0 equiv.) and 2-chloro-5-(trifluoromethyl)pyridine (52 mg, 285 μmol, 1.1 equiv.) as the starting materials to give 6-(1-(2,2-difluoroethyl)-1H-pyrazolo[3,4-b]pyrazin-6-yl)-2-(5-(trifluoromethyl)pyridin-2-yl)-2,6-diazaspiro[3.4]octan-5-one (45.7 mg, 38%) as a white solid. ¹H NMR (400 MHz, DMSO-d₆) δ 9.70 (s, 1H), 8.50 (s, 1H), 8.42 (dt, J=2.1, 1.0 Hz, 1H), 7.83 (dd, J=8.9, 2.5 Hz, 1H), 6.60-6.49 (m, 2H), 4.88 (td, J=15.1, 3.6 Hz, 2H), 4.29 (d, J=8.7 Hz, 2H), 4.12 (d, J=8.7 Hz, 2H), 4.09-4.03 (m, 2H), 2.59 (t, J=7.0 Hz, 2H). MS m/z: 454.00 [M+H]⁺.

### Example 1012: 6-(1-(2,2-difluoroethyl)-1H-pyrazolo[3,4-b]pyrazin-6-yl)-2-(2-(trifluoromethyl)pyrimidin-5-yl)-2,6-diazaspiro[3.4]octan-5-one

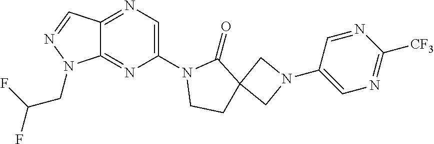 [SMILES: O=C1N(c2cnc3cnn(CC(F)F)c3n2)CCC12CN(c1cnc(C(F)(F)F)nc1)C2]

Step 1: 6-(1-(2,2-difluoroethyl)-1H-pyrazolo[3,4-b]pyrazin-6-yl)-2-(2-(trifluoromethyl)pyrimidin-5-yl)-2,6-diazaspiro[3.4]octan-5-one: Followed the general procedure Y using 6-(1-(2,2-difluoroethyl)-1H-pyrazolo[3,4-b]pyrazin-6-yl)-2,6-diazaspiro[3.4]octan-5-one (80 mg, 258 μmol, 1.0 equiv.) and 5-bromo-2-(trifluoromethyl)pyrimidine (226 mg, 284 μmol, 1.1 equiv.) as the starting materials, Ruphos Pd G3 (22 mg, 25 μmol, 0.1 equiv.) as the catalyst to give 6-(1-(2,2-difluoroethyl)-1H-pyrazolo[3,4-b]pyrazin-6-yl)-2-(2-(trifluoromethyl)pyrimidin-5-yl)-2,6-diazaspiro[3.4]octan-5-one (20.4 mg, 17%) as a white solid. ¹H NMR (400 MHz, DMSO-d₆) δ 9.71 (s, 1H), 8.51 (s, 1H), 8.24 (s, 2H), 6.65-6.38 (m, 1H), 4.89 (td, J=15.2, 3.6 Hz, 2H), 4.33 (d, J=8.3 Hz, 2H), 4.17 (d, J=8.4 Hz, 2H), 4.06 (dd, J=8.2, 5.8 Hz, 2H), 2.60 (t, J=7.0 Hz, 2H). MS m/z: 455.00 [M+H]⁺.

### Example 1013: 1-(2,2-difluoroethyl)-5-methyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one

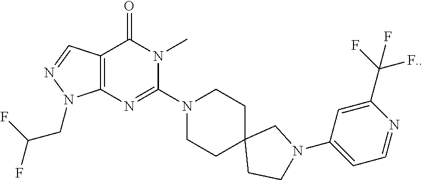 [SMILES: Cn1c(N2CCC3(CCN(c4ccnc(C(F)(F)F)c4)C3)CC2)nc2c(cnn2CC(F)F)c1=O]

Step 1: tert-butyl 2-(2-(trifluoromethyl)pyridin-4-yl)-2,8-diazaspiro[4.5]decane-8-carboxylate: Followed the general procedure I using tert-butyl 2,8-diazaspiro[4.5]decane-8-carboxylate (100 mg, 0.416 mmol, 1.0 equiv.) and 4-bromo-2-(trifluoromethyl)pyridine (141 mg, 0.624 mmol, 1.5 equiv.) as the starting materials to give tert-butyl 2-(2-(trifluoromethyl)pyridin-4-yl)-2,8-diazaspiro[4.5]decane-8-carboxylate (100 mg, 62%) as a white solid. MS m/z: 386 [M+H]⁺.

Step 2: 2-(2-(trifluoromethyl)pyridin-4-yl)-2,8-diazaspiro[4.5]decane hydrochloride: Followed the general procedure B using tert-butyl 2-(2-(trifluoromethyl)pyridin-4-yl)-2,8-diazaspiro[4.5]decane-8-carboxylate (100 mg) as the starting material to give the crude product 2-(2-(trifluoromethyl)pyridin-4-yl)-2,8-diazaspiro[4.5]decane hydrochloride (100 mg). MS m/z: 286 [M+H]⁺

Step 3: 1-(2,2-difluoroethyl)-5-methyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure I using 6-chloro-1-(2,2-difluoroethyl)-5-methyl-1,5-dihydro-4H-pyrazolo[3,4-d]pyrimidin-4-one (60 mg, 0.241 mmol, 1.0 equiv.) and 2-(2-(trifluoromethyl)pyridin-4-yl)-2,8-diazaspiro[4.5]decane hydrochloride (77.6 mg, 0.241 mmol, 1.0 equiv.) as the starting materials to give 1-(2,2-difluoroethyl)-5-methyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (96 mg, 78%) as a white solid. ¹H NMR (400 MHz, DMSO-d₆) δ 8.21 (d, J=5.8 Hz, 1H), 8.02 (s, 1H), 6.88 (s, 1H), 6.69 (dd, J=5.9, 2.3 Hz, 1H), 6.44 (tt, J=54.9, 3.9 Hz, 1H), 4.63 (td, J=14.9, 3.9 Hz, 2H), 3.49-3.41 (m, 5H), 3.40-3.33 (m, 4H), 3.29-3.19 (m, 2H), 1.96 (t, J=7.0 Hz, 2H), 1.79-1.69 (m, 4H). MS m/z: 498.2 [M+H]⁺.

### Example 1014: 1-(2,2-difluoroethyl)-5-methyl-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one

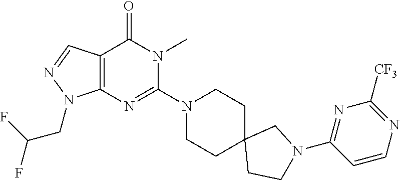 [SMILES: Cn1c(N2CCC3(CCN(c4ccnc(C(F)(F)F)n4)C3)CC2)nc2c(cnn2CC(F)F)c1=O]

Step 1: tert-butyl 2-(2-(trifluoromethyl)pyrimidin-4-yl)-2,8-diazaspiro[4.5]decane-8-carboxylate: Followed the general procedure I using tert-butyl 2,8-diazaspiro[4.5]decane-8-carboxylate (200 mg, 0.83 mmol, 1.0 equiv.), and 4-chloro-2-(trifluoromethyl)pyrimidine (152 mg, 0.83 mmol, 1.0 equiv.) as the starting materials to give tert-butyl 2-(2-(trifluoromethyl)pyrimidin-4-yl)-2,8-diazaspiro[4.5]decane-8-carboxylate (195 mg, 61%) as a white solid. MS m/z: 387 [M+H]⁺.

Step 2: 2-(2-(trifluoromethyl)pyrimidin-4-yl)-2,8-diazaspiro[4.5]decane hydrochloride: Followed the general procedure B using tert-butyl 2-(2-(trifluoromethyl)pyrimidin-4-yl)-2,8-diazaspiro[4.5]decane-8-carboxylate (195 mg) as the starting material to give the crude product 2-(2-(trifluoromethyl)pyrimidin-4-yl)-2,8-diazaspiro[4.5]decane hydrochlorid (180 mg). MS m/z: 287 [M+H]⁺.

Step 3: 1-(2,2-difluoroethyl)-5-methyl-6-(2-(2-trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure I using 2-(2-(trifluoromethyl)pyrimidin-4-yl)-2,8-diazaspiro[4.5]decane hydrochloride (57.6 mg, 0.2 mmol, 1 equiv.) and 6-chloro-1-(2,2-difluoroethyl)-5-methyl-1,5-dihydro-4H-pyrazolo[3,4-d]pyrimidin-4-one (50 mg, 0.2 mmol, 1.0 equiv.) as the starting materials to give 1-(2,2-difluoroethyl)-5-methyl-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one (64.2 mg, 63%) as a white solid. ¹H NMR (300 MHz, DMSO-d₆) δ 8.30 (d, J=6.1 Hz, 1H), 8.02 (s, 1H), 6.82-6.69 (m, 1H), 6.66-6.22 (m, 1H), 4.75-4.52 (m, 2H), 3.63 (t, J=7.1 Hz, 1H), 3.50 (d, J=12.4 Hz, 2H), 3.41 (d, J=10.7 Hz, 4H), 2.05-1.87 (m, 2H), 1.74 (s, 4H). MS m/z: 499 [M+H]⁺.

### Example 1015: 1-(2,2-difluoroethyl)-5-methyl-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one

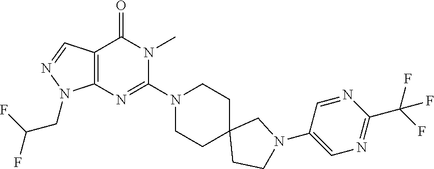 [SMILES: Cn1c(N2CCC3(CCN(c4cnc(C(F)(F)F)nc4)C3)CC2)nc2c(cnn2CC(F)F)c1=O]

Step 1: tert-butyl 2-(2-(trifluoromethyl)pyrimidin-5-yl)-2,8-diazaspiro[4.5]decane-8-carboxylate: Followed the general procedure Y using tert-butyl 2,8-diazaspiro[4.5]decane-8-carboxylate (300 mg, 1.25 mmol, 1.0 equiv.) and 5-bromo-2-(trifluoromethyl)pyrimidine (283 mg, 1.25 mmol, 1.0 equiv.) as the starting materials to give tert-butyl 2-(2-(trifluoromethyl)pyrimidin-5-yl)-2,8-diazaspiro[4.5]decane-8-carboxylate (480 mg, 60%) as a white solid. MS m/z: 387 [M+H]⁺.

Step 2: 2-(2-(trifluoromethyl)pyrimidin-5-yl)-2,8-diazaspiro[4.5]decane: Followed the general procedure H using tert-butyl 2-(2-(trifluoromethyl)pyrimidin-5-yl)-2,8-diazaspiro[4.5]decane-8-carboxylate (100 mg) as the starting material to give the crude product 2-(2-(trifluoromethyl)pyrimidin-5-yl)-2,8-diazaspiro[4.5]decane (100 mg). MS m/z: 287 [M+H]⁺.

Step 3: 1-(2,2-difluoroethyl)-5-methyl-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one: Followed the general procedure Y using 2-(2-(trifluoromethyl)pyrimidin-5-yl)-2,8-diazaspiro[4.5]decane (100 mg, 0.348 mmol, 1.0 equiv.) and 6-chloro-1-(2,2-difluoroethyl)-5-methyl-1,5-dihydro-4H-pyrazolo[3,4-d]pyrimidin-4-one (87 mg, 0.349 mmol, 1.0 equiv.) as the starting materials, EPhos Pd G4 (32.1 mg, 0.035 mmol, 0.1 equiv.) as the catalyst to give 1-(2,2-difluoroethyl)-5-methyl-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one (35 mg, 35%) as a white solid. ¹H NMR (300 MHz, DMSO-d₆) δ 8.25 (s, 2H), 8.03 (s, 1H), 6.43 (tt, 1H), 4.62 (td, 2H), 3.49 (t, 2H), 3.49-3.18 (m, 9H), 1.98 (t, 2H), 1.74 (t, 4H). MS m/z: 499.2 [M+H]⁺.

### Example 1016: 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-7-one

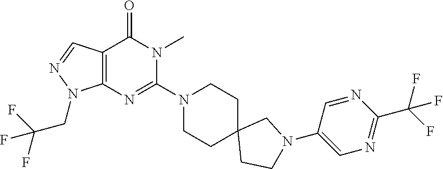 [SMILES: Cn1c(N2CCC3(CCN(c4cnc(C(F)(F)F)nc4)C3)CC2)nc2c(cnn2CC(F)(F)F)c1=O]

Step 1: 2-(2-(trifluoromethyl)pyrimidin-5-yl)-2,8-diazaspiro[4.5]decane: Followed the general procedure H using tert-butyl 2-(2-(trifluoromethyl)pyrimidin-5-yl)-2,8-diazaspiro[4.5]decane-8-carboxylate (450 mg) as the starting material to give the crude product 2-(2-(trifluoromethyl)pyrimidin-5-yl)-2,8-diazaspiro[4.5]decane (450 mg). MS m/z: 287 [M+H]⁺.

Step 2: 5-methyl-1-(tetrahydro-2H-pyran-2-yl)-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one: Followed the general procedure I using 2-(2-(trifluoromethyl)pyrimidin-5-yl)-2,8-diazaspiro[4.5]decane (100 mg, 414 μmol, 1.0 equiv.) and 6-chloro-5-methyl-1-(tetrahydro-2H-pyran-2-yl)-1,5-dihydro-4H-pyrazolo[3,4-d]pyrimidin-4-one (94 mg, 0.349 mmol, 1.0 equiv.) as the starting materials to give 5-methyl-1-(tetrahydro-2H-pyran-2-yl)-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one (130 mg, 60%) as a white solid. MS m/z: 519 [M+H]⁺.

Step 3: 5-methyl-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one: Followed the general procedure M using 5-methyl-1-(tetrahydro-2H-pyran-2-yl)-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one (130 mg, 0.251 mmol, 1.0 equiv.) as the starting material to give 5-methyl-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one (90 mg, 70%) as a white solid. MS m/z: 435 [M+H]⁺.

Step 4: 5-methyl-1-(2,2,2-trifluoroethyl)-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one: Followed the general procedure K using 5-methyl-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one (90 mg, 0.207 mmol, 1.0 equiv.) and 2,2,2-trifluoroethyl trifluoromethanesulfonate (240 mg, 1.03 mmol, 5.0 equiv.) as the starting materials to give 5-methyl-1-(2,2,2-trifluoroethyl)-6-(2-(2-(trifluoromethyl)pyrimidin-5-yl)-2,8-diazaspiro[4.5]decan-8-yl)-1,5-dihydro-4H-pyrazolo[3,4-d]pyrimidin-4-one (6 mg, 20%) as a white solid. ¹H NMR (300 MHz, Chloroform-d) δ 8.11 (d, J=16.6 Hz, 3H), 4.84 (q, 2H), 3.61-3.51 (m, 5H), 3.41-3.21 (m, 6H), 2.11 (t, 2H), 1.87 (t, J=5.7 Hz, 4H). MS m/z: 517.05 [M+H]⁺.

### Example 1017: 5-methyl-1-(2,2,2-trifluoroethyl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one

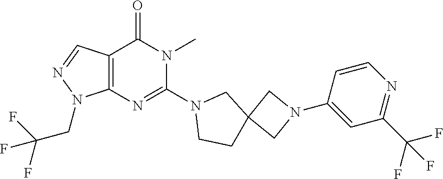 [SMILES: Cn1c(N2CCC3(CN(c4ccnc(C(F)(F)F)c4)C3)C2)nc2c(cnn2CC(F)(F)F)c1=O]

Step 1: 5-methyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure I using 2-(2-(trifluoromethyl)pyridin-4-yl)-2,6-diazaspiro[3.4]octane hydrochloride (200 mg, 0.681 mmol, 1.00 equiv) and 6-chloro-5-methyl-1-(tetrahydro-2H-pyran-2-yl)-1,5-dihydro-4H-pyrazolo[3,4-d]pyrimidin-4-one (183 mg, 0.681 mmol, 1.00 equiv) as the starting materials to give 5-methyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (280 mg, 84.0%) as a light yellow oil. MS m/z: 490 [M+H]⁺.

Step 2: 5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure H using 5-methyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (280 mg, 0.531 mmol, 1.00 equiv) as the starting material to give the crude product 5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (160 mg). MS m/z: 406 [M+H]⁺.

Step 3: 5-methyl-1-(2,2,2-trifluoroethyl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure K using 5-methyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (80.0 mg, 0.197 mmol, 1.00 equiv) and 2,2,2-trifluoroethyl trifluoromethanesulfonate (229 mg, 0.985 mmol, 5.00 equiv) as the starting materials to give 5-methyl-1-(2,2,2-trifluoroethyl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (17.1 mg, 17.8%) as a white solid. ¹H NMR (400 MHz, Chloroform-d) δ 8.35 (d, 1H), 8.02 (s, 1H), 6.63 (d, 1H), 6.41-6.38 (m, 1H), 4.84-4.74 (m, 2H), 4.10-4.01 (m, 4H), 3.83 (s, 2H), 3.69 (t, 2H), 3.52 (s, 3H), 2.31 (t, 2H). MS m/z: 499.1 [M+H]⁺.

### Example 1018: 6-(1-(oxetan-3-yl)-1H-pyrazolo[3,4-d]pyrimidin-6-yl)-2-(2-(trifluoromethyl)pyridin-4-yl)-2,6-diazaspiro[3.4]octan-7-one

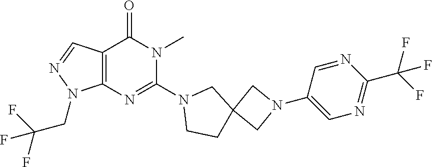 [SMILES: Cn1c(N2CCC3(CN(c4cnc(C(F)(F)F)nc4)C3)C2)nc2c(cnn2CC(F)(F)F)c1=O]

Step 1: 5-methyl-1-(tetrahydro-2H-pyran-2-yl)-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one: Followed the general procedure I using 6-chloro-5-methyl-1-(tetrahydro-2H-pyran-2-yl)-1,5-dihydro-4H-pyrazolo[3,4-d]pyrimidin-4-one (100 mg, 0.372 mmol, 1.0 equiv.) and 2-(2-(trifluoromethyl)pyrimidin-5-yl)-2,6-diazaspiro[3.4]octane (96 mg, 0.372 mmol, 1.0 equiv.) as the starting materials to give (100 mg, 60%) as a white solid. MS m/z: 491 [M+H]⁺.

Step 2: 5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one: Followed the general procedure M using 5-methyl-1-(tetrahydro-2H-pyran-2-yl)-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (130 mg, 0.265 mmol, 1.0 equiv.) as the starting material to give 5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (80.0 mg, 60%) as a white solid. MS m/z: 407 [M+H]⁺.

Step 3: 5-methyl-1-(2,2,2-trifluoroethyl)-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one: Followed the general procedure K using 5-methyl-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (80.0 mg, 0.197 mmol, 1.0 equiv.) and 2,2,2-trifluoroethyl trifluoromethanesulfonate (50.2 mg, 0.216 mmol, 1.1 equiv.) as the starting materials to give 5-methyl-1-(2,2,2-trifluoroethyl)-6-(2-(2-(trifluoromethyl)pyrimidin-5-yl)-2,6-diazaspiro[3.4]octan-6-yl)-1,5-dihydro-4H-pyrazolo[3,4-d]pyrimidin-4-one (20 mg, 30%) as a white solid. ¹H NMR (300 MHz, Chloroform-d) δ 8.02 (s, 3H), 4.79 (q, 2H), 4.12 (q, 4H), 3.86 (s, 2H), 3.70 (t, 2H), 3.53 (s, 3H), 2.34 (t, 2H). MS m/z: 489.05 [M+H]⁺.

### Example 1019: 8-(1-(2,2-difluoroethyl)-5-ethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one

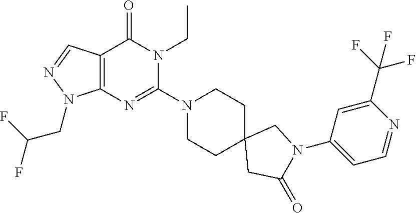 [SMILES: CCn1c(N2CCC3(CC2)CC(=O)N(c2ccnc(C(F)(F)F)c2)C3)nc2c(cnn2CC(F)F)c1=O]

Step 1: tert-butyl 3-oxo-2-(2-(trifluoromethyl)pyridin-4-yl)-2,8-diazaspiro[4.5]decane-8-carboxylate: Followed the general procedure Y using tert-butyl 3-oxo-2,8-diazaspiro[4.5]decane-8-carboxylate (100 mg, 0.394 mmol, 1.0 equiv.) and 4-chloro-2-(trifluoromethyl)pyridine (71.7 mg, 0.394 mmol, 1.0 equiv.) as the starting materials, XPhos Pd G3 (37.6 mg, 0.04 mmol, 0.1 equiv.) as the catalyst to give tert-butyl 3-oxo-2-(2-(trifluoromethyl)pyridin-4-yl)-2,8-diazaspiro[4.5]decane-8-carboxylate (140 mg, 80%) as a white solid. MS m/z: 400 [M+H]⁺.

Step 2: 2-(2-(trifluoromethyl)pyridin-4-yl)-2,8-diazaspiro[4.5]decan-3-one: Followed the general procedure H using tert-butyl 3-oxo-2-(2-(trifluoromethyl)pyridin-4-yl)-2,8-diazaspiro[4.5]decane-8-carboxylate (140 mg) as the starting material to give the crude product 2-(2-(trifluoromethyl)pyridin-4-yl)-2,8-diazaspiro[4.5]decan-3-one (140 mg). MS m/z: 300 [M+H]⁺.

Step 3: 8-(5-ethyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure I using 2-(2-(trifluoromethyl)pyridin-4-yl)-2,8-diazaspiro[4.5]decan-3-one (140 mg, 0.467 mmol, 1.0 equiv.) and 6-chloro-5-ethyl-1-(tetrahydro-2H-pyran-2-yl)-1,5-dihydro-4H-pyrazolo[3,4-d]pyrimidin-4-one (132 mg, 0.467 mmol, 1.0 equiv.) as the starting materials to give 8-(5-ethyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one (120 mg, 60%) as a white solid. MS m/z: 546 [M+H]⁺.

Step 4: 8-(5-ethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure M using 8-(5-ethyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one (120 mg, 0.220 mmol, 1.0 equiv.) as the starting material to give 8-(5-ethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one (100 mg, 90%) as a white solid. MS m/z: 462 [M+H]⁺.

Step 5: 8-(1-(2,2-difluoroethyl)-5-ethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure K using 8-(5-ethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one (100 mg, 0.216 mmol, 1.0 equiv.) and CHF₂CH₂OTf (138 mg, 0.649 mmol, 3.0 equiv.) as the starting materials to give 8-(1-(2,2-difluoroethyl)-5-ethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one (24.5 mg, 30%) as a white solid. ¹H NMR (300 MHz, DMSO-d₆) δ 8.69 (d, 1H), 8.28 (d, 1H), 8.04 (s, 1H), 7.92 (dd, 1H), 6.5 (tt, 1H), 4.6 (td, 2H), 4.03 (q, 2H), 3.87 (s, 2H), 3.29-3.05 (m, 4H), 2.65 (d, 2H), 1.81 (d, 4H), 1.19 (q, 3H). MS m/z: 526.2 [M+H]⁺.

### Example 1020: 8-(1-(2,2-difluoroethyl)-3,5-dimethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one

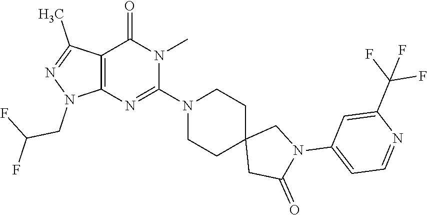 [SMILES: Cc1nn(CC(F)F)c2nc(N3CCC4(CC3)CC(=O)N(c3ccnc(C(F)(F)F)c3)C4)n(C)c(=O)c12]

Step 1: 4,6-dichloro-3-iodo-1H-pyrazolo[3,4-d]pyrimidine: Followed the general procedure V using 4,6-dichloro-1H-pyrazolo[3,4-d]pyrimidine (5 g, 26.5 mmol, 1.0 equiv.) as the starting material, NIS (11.9 g, 52.9 mmol, 2.0 equiv.) as the reagent to give 4,6-dichloro-3-iodo-1H-pyrazolo[3,4-d]pyrimidine (5 g, 60%) as a white solid. MS m/z: 315 [M+H]⁺.

Step 2: 4,6-dichloro-3-iodo-1-(tetrahydro-2H-pyran-2-yl)-1H-pyrazolo[3,4-d]pyrimidine. A solution of 4,6-dichloro-3-iodo-1H-pyrazolo[3,4-d]pyrimidine (4.5 g, 14.3 mmol, 1.0 equiv.) and TsOH (0.25 g, 1.43 mmol, 0.1 equiv.) in DCM (20 mL) was stirred for 2 h at room temperature under air atmosphere. Desired product could be detected by LCMS. The resulting mixture was concentrated under reduced pressure. The residue was purified by silica gel column chromatography, eluted with PE/EA (1:1) to afford 4,6-dichloro-3-iodo-1-(tetrahydro-2H-pyran-2-yl)-1H-pyrazolo[3,4-d]pyrimidine (4.5 g, 79%) as a white solid. MS m/z: 399 [M+H]⁺.

Step 3: 6-chloro-3-iodo-1-(tetrahydro-2H-pyran-2-yl)-1H-pyrazolo[3,4-d]pyrimidin-4-ol. A solution of 4,6-dichloro-3-iodo-1-(tetrahydro-2H-pyran-2-yl)-1H-pyrazolo[3,4-d]pyrimidine (4.5 g, 11.3 mmol, 1.0 equiv.) and KOH (17.2 g, 306 mmol, 27.2 equiv.) in H₂O (100 mL) was stirred for 2 h at 60° C. under air atmosphere. Desired product could be detected by LCMS. The mixture was acidified neutralized to pH 3 with HCl (aq.). The precipitated solids were collected by filtration and washed with MeCN (3×10 mL). This resulted in 6-chloro-3-iodo-1-(tetrahydro-2H-pyran-2-yl)-1H-pyrazolo[3,4-d]pyrimidin-4-ol (3.8 g, 88%) as a white solid. MS m/z: 381 [M+H]⁺.

Step 4: 6-chloro-3-iodo-5-methyl-1-(tetrahydro-2H-pyran-2-yl)-1,5-dihydro-4H-pyrazolo[3,4-d]pyrimidin-4-one: Followed the general procedure K using 6-chloro-3-iodo-1-(tetrahydro-2H-pyran-2-yl)-1H-pyrazolo[3,4-d]pyrimidin-4-ol (3.8 g, 9.98 mmol, 1.0 equiv.) and Mel (2.83 g, 20 mmol, 2.0 equiv.) as the starting materials to give 6-chloro-3-iodo-5-methyl-1-(tetrahydro-2H-pyran-2-yl)-1,5-dihydro-4H-pyrazolo[3,4-d]pyrimidin-4-one (3.8 g, 96%) as a white solid. MS m/z: 395 [M+H]⁺.

Step 5: tert-butyl 3-oxo-2-(2-(trifluoromethyl)pyridin-4-yl)-2,8-diazaspiro[4.5]decane-8-carboxylate: Followed the general procedure Y using tert-butyl 3-oxo-2,8-diazaspiro[4.5]decane-8-carboxylate (300 mg, 1.18 mmol, 1.0 equiv.) and 4-bromo-2-(trifluoromethyl)pyridine (533 mg, 2.36 mmol, 2.0 equiv.) as the starting materials to give tert-butyl 3-oxo-2-(2-(trifluoromethyl)pyridin-4-yl)-2,8-diazaspiro[4.5]decane-8-carboxylate (370 mg, 79%) as a white solid. MS m/z: 400 [M+H]⁺.

Step 6: 2-(2-(trifluoromethyl)pyridin-4-yl)-2,8-diazaspiro[4.5]decan-3-one hydrochloride: Followed the general procedure B using tert-butyl 3-oxo-2-(2-(trifluoromethyl)pyridin-4-yl)-2,8-diazaspiro[4.5]decane-8-carboxylate (370 mg) as the starting material to give the crude product 2-(2-(trifluoromethyl)pyridin-4-yl)-2,8-diazaspiro[4.5]decan-3-one hydrochloride (280 mg). MS m/z: 300 [M+H]⁺.

Step 7: 8-(3-iodo-5-methyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure I using 2-(2-(trifluoromethyl)pyridin-4-yl)-2,8-diazaspiro[4.5]decan-3-one hydrochloride (260 mg, 869 μmol, 1.0 equiv.) and 6-chloro-3-iodo-5-methyl-1-(tetrahydro-2H-pyran-2-yl)-1,5-dihydro-4H-pyrazolo[3,4-d]pyrimidin-4-one (411 mg, 1.04 mmol, 1.2 equiv.) as the starting materials to give 8-(3-iodo-5-methyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one (490 mg, 86%) as a white solid. MS m/z: 658 [M+H]⁺.

Step 8: 8-(3,5-dimethyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure L using 8-(3-iodo-5-methyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one (469 mg, 713 μmol, 1.0 equiv.) and methylboronic acid (85.4 mg, 1.43 mmol, 2.0 equiv.) as the starting materials to give 8-(3,5-dimethyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one (280 mg, 72%) as a white solid. MS m/z: 546 [M+H]⁺.

Step 9: 8-(3,5-dimethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure M using 8-(3,5-dimethyl-4-oxo-1-(tetrahydro-2H-pyran-2-yl)-4,5-dihydro-1H-pyrazolo[3,4-d]pyrimidin-6-yl)-2-(2-(trifluoromethyl)pyridin-4-yl)-2,8-diazaspiro[4.5]decan-3-one (270 mg, 495 μmol, 1.0 equiv.) as the starting material to give 8-(3,5-dimethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one (120 mg, 53%) as a white solid. MS m/z: 462 [M+H]⁺.

Step 10: 8-(1-(2,2-difluoroethyl)-3,5-dimethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one: Followed the general procedure K using 8-(3,5-dimethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one (110 mg, 238 μmol, 1.0 equiv.) and 2,2-difluoroethyl trifluoromethanesulfonate (102 mg, 476 μmol, 2.0 equiv.) as the starting materials to give 8-(1-(2,2-difluoroethyl)-3,5-dimethyl-4-oxo-4,5-dihydro-1H-pyrazolo[3,4-d]pyrimidin-6-yl)-2-(2-(trifluoromethyl)pyridin-4-yl)-2,8-diazaspiro[4.5]decan-3-one (53.6 mg, 42%) as a white solid. ¹H NMR (400 MHz, DMSO-d₆) δ 8.69 (d, J=5.6 Hz, 1H), 8.28 (d, J=2.1 Hz, 1H), 7.92 (dd, J=5.7, 2.1 Hz, 1H), 6.60-6.17 (m, 1H), 4.54 (td, J=14.8, 3.9 Hz, 2H), 3.87 (s, 2H), 3.40 (s, 3H), 3.28 (s, 4H), 2.65 (s, 2H), 2.39 (s, 3H), 1.97-1.68 (m, J=7.4, 6.3 Hz, 4H). MS m/z: 526.19 [M+H]⁺.

### Example 1021: 2-(3-methoxy-6-(2-(2-(trifluoromethyl)pyridin-4-yl)-2,6-diazaspiro[3.4]octan-6-yl)pyrazin-2-yl)-1,3,4-thiadiazole

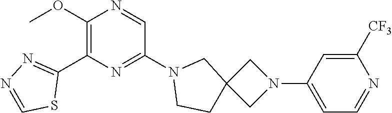 [SMILES: COc1ncc(N2CCC3(CN(c4ccnc(C(F)(F)F)c4)C3)C2)nc1-c1nncs1]

Step 1: methyl 6-bromo-3-methoxypyrazine-2-carboxylate. A mixture of methyl 6-bromo-3-chloropyrazine-2-carboxylate (2 g, 7.97 mmol, 1.0 equiv.) and sodium methanolate (526 mg, 9.57 mmol, 1.2 equiv.) in MeOH (13 mL) was stirred for 2 h at 100° C. under air atmosphere. After filtration, the filtrate was concentrated under reduced pressure. The residue was purified by reverse flash chromatography with the following conditions (column, C18 gel; mobile phase, B phase: MeCN, A phase: water; 5% to 100% B gradient in 20 min; detector: UV 254/220 nm). This resulted in methyl 6-bromo-3-methoxypyrazine-2-carboxylate (650 mg, 32%) as a white solid. MS m/z: 247 [1H]⁺.

Step 2: tert-butyl 6-(5-methoxy-6-(methoxycarbonyl)pyrazin-2-yl)-2,6-diazaspiro[3.4]octane-2-carboxylate: Followed the general procedure Y using methyl 6-bromo-3-methoxypyrazine-2-carboxylate (650 mg, 2.63 mmol, 1.0 equiv.) and tert-butyl 2,6-diazaspiro[3.4]octane-2-carboxylate (673 mg, 3.16 mmol, 1.2 equiv.) as the starting materials, to give tert-butyl 6-(5-methoxy-6-(methoxycarbonyl)pyrazin-2-yl)-2,6-diazaspiro[3.4]octane-2-carboxylate (550 mg, 55%) as a white solid. MS m/z: 379 [M+H]⁺.

Step 3: tert-butyl 6-(6-(hydrazinecarbonyl)-5-methoxypyrazin-2-yl)-2,6-diazaspiro[3.4]octane-2-carboxylate: Followed the general procedure S using tert-butyl 6-(5-methoxy-6-(methoxycarbonyl)pyrazin-2-yl)-2,6-diazaspiro[3.4]octane-2-carboxylate (550 mg, 1.58 mmol, 1.0 equiv.) as the starting material to give tert-butyl 6-(6-(hydrazinecarbonyl)-5-methoxypyrazin-2-yl)-2,6-diazaspiro[3.4]octane-2-carboxylate (500 mg, 90%) as a white solid. MS m/z: 379 [M+H]⁺.

Step 4: tert-butyl 6-(6-(2-formylhydrazine-1-carbonyl)-5-methoxypyrazin-2-yl)-2,6-diazaspiro[3.4]octane-2-carboxylate: Followed the general procedure AF using tert-butyl 6-(6-(hydrazinecarbonyl)-5-methoxypyrazin-2-yl)-2,6-diazaspiro[3.4]octane-2-carboxylate (500 mg, 1.32 mmol, 1.0 equiv.) as the starting material to give tert-butyl 6-(6-(2-formylhydrazine-1-carbonyl)-5-methoxypyrazin-2-yl)-2,6-diazaspiro[3.4]octane-2-carboxylate (520 mg, 96%) as a yellow oil. MS m/z: 407 [M+H]⁺.

Step 5: tert-butyl 6-(5-methoxy-6-(1,3,4-thiadiazol-2-yl)pyrazin-2-yl)-2,6-diazaspiro[3.4]octane-2-carboxylate: Followed the general procedure T using tert-butyl 6-(6-(2-formylhydrazine-1-carbonyl)-5-methoxypyrazin-2-yl)-2,6-diazaspiro[3.4]octane-2-carboxylate (520 mg, 1.28 mmol, 1.0 equiv.) as the starting material to give tert-butyl 6-(5-methoxy-6-(1,3,4-thiadiazol-2-yl)pyrazin-2-yl)-2,6-diazaspiro[3.4]octane-2-carboxylate (300 mg, 58%) as a yellow green solid. MS m/z: 405 [M+H]⁺.

Step 6: 2-(6-(2,6-diazaspiro[3.4]octan-6-yl)pyrazin-2-yl)-5-(trifluoromethyl)-1,3,4-oxadiazole: Followed the general procedure B using tert-butyl 6-(6-(5-(trifluoromethyl)-1,3,4-thiadiazol-2-yl)pyrazin-2-yl)-2,6-diazaspiro[3.4]octane-2-carboxylate (300 mg) as the starting material to give the crude product 2-(3-methoxy-6-(2,6-diazaspiro[3.4]octan-6-yl)pyrazin-2-yl)-1,3,4-thiadiazole (200 mg) as a white solid. MS m/z: 305 [M+H]⁺.

Step 7: 2-(trifluoromethyl)-5-(6-(2-(2-(trifluoromethyl)pyrimidin-5-yl)-2,6-diazaspiro[3.4]octan-6-yl)pyrazin-2-yl)-1,3,4-thiadiazole: Followed the general procedure I using 2-(3-methoxy-6-(2,6-diazaspiro[3.4]octan-6-yl)pyrazin-2-yl)-1,3,4-thiadiazole (100 mg, 0.328 mmol, 1.0 equiv.) and 4-chloro-2-(trifluoromethyl)pyridine (71.6 mg, 0.394 mmol, 1.2 equiv.) as the starting materials to give 2-(3-methoxy-6-(2-(2-(trifluoromethyl)pyridin-4-yl)-2,6-diazaspiro[3.4]octan-6-yl)pyrazin-2-yl)-1,3,4-thiadiazole (51.7 mg, 35%) as a yellow solid. ¹H NMR (400 MHz, DMSO-d₆) δ 9.68 (s, 1H), 8.25 (d, J=5.7 Hz, 1H), 7.85 (s, 1H), 6.76 (d, J=2.3 Hz, 1H), 6.58 (dd, J=5.8, 2.3 Hz, 1H), 4.08-4.00 (m, 4H), 3.99 (s, 3H), 3.74 (s, 2H), 3.55 (t, J=6.9 Hz, 2H), 2.30 (t, J=6.8 Hz, 2H). MS m/z: 450 [M+H]⁺.

### Example 1022: 2-(3-methoxy-6-(2-(2-(trifluoromethyl)pyrimidin-5-yl)-2,6-diazaspiro[3.4]octan-6-yl)pyrazin-2-yl)-1,3,4-thiadiazole

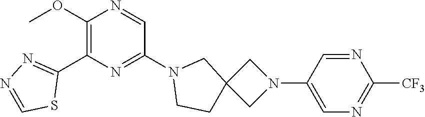 [SMILES: COc1ncc(N2CCC3(CN(c4cnc(C(F)(F)F)nc4)C3)C2)nc1-c1nncs1]

Step 1: 2-(3-methoxy-6-(2-(2-(trifluoromethyl)pyrimidin-5-yl)-2,6-diazaspiro[3.4]octan-6-yl)pyrazin-2-yl)-1,3,4-thiadiazole: Followed the general procedure Y using 2-(3-methoxy-6-(2,6-diazaspiro[3.4]octan-6-yl)pyrazin-2-yl)-1,3,4-thiadiazole (100 mg, 0.328 mmol, 1.0 equiv.) and 5-bromo-2-(trifluoromethyl)pyrimidine (89.4 mg, 0.394 mmol, 1.2 equiv.) as the starting materials to give 2-(3-methoxy-6-(2-(2-(trifluoromethyl)pyrimidin-5-yl)-2,6-diazaspiro[3.4]octan-6-yl)pyrazin-2-yl)-1,3,4-thiadiazole (28.6 mg, 19%) as a yellow solid. ¹H NMR (400 MHz, DMSO-d₆) δ 9.68 (s, 1H), 8.18 (s, 2H), 7.85 (s, 1H), 4.14-4.07 (m, 4H), 3.99 (s, 3H), 3.75 (s, 2H), 3.58-3.52 (m, 2H), 2.36-2.29 (m, 2H). MS m/z: 450.95 [M+H]⁺.

### Example 1023: 2-(trifluoromethyl)-5-(6-(2-(2-(trifluoromethyl)pyridin-4-yl)-2,6-diazaspiro[3.4]octan-6-yl)pyrazin-2-yl)-1,3,4-thiadiazole

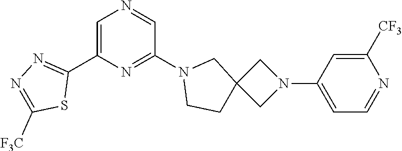 [SMILES: FC(F)(F)c1cc(N2CC3(CCN(c4cncc(-c5nnc(C(F)(F)F)s5)n4)C3)C2)ccn1]

Step 1: tert-butyl 6-(6-(methoxycarbonyl)pyrazin-2-yl)-2,6-diazaspiro[3.4]octane-2-carboxylate: Followed the general procedure I using 6-chloropyrazine-2-carboxylate (500 mg, 2.89 mmol, 1.0 equiv.) and tert-butyl 2,6-diazaspiro[3.4]octane-2-carboxylate (739 mg, 3.47 mmol, 1.1 equiv.) as the starting materials to give tert-butyl 6-(6-(methoxycarbonyl)pyrazin-2-yl)-2,6-diazaspiro[3.4]octane-2-carboxylate (500 mg, 49%) as a white solid. MS m/z: 349 [M+H]⁺.

Step 2: tert-butyl 6-(6-(hydrazinecarbonyl)pyrazin-2-yl)-2,6-diazaspiro[3.4]octane-2-carboxylate: Followed the general procedure S using tert-butyl 6-(6-(methoxycarbonyl)pyrazin-2-yl)-2,6-diazaspiro[3.4]octane-2-carboxylate (500 mg, 1.44 mmol, 1.0 equiv.) as the starting material to give tert-butyl 6-(6-(hydrazinecarbonyl)pyrazin-2-yl)-2,6-diazaspiro[3.4]octane-2-carboxylate (450 mg, 90%) as a white solid. MS m/z: 349 [M+H]⁺

Step 3: tert-butyl 6-(6-(2-(2,2,2-trifluoroacetyl)hydrazine-1-carbonyl)pyrazin-2-yl)-2,6-diazaspiro[3.4]octane-2-carboxylate: Followed the general procedure AF using tert-butyl 6-(6-(hydrazinecarbonyl)pyrazin-2-yl)-2,6-diazaspiro[3.4]octane-2-carboxylate (410 mg, 1.18 mmol, 1.0 equiv.) as the starting material to give tert-butyl 6-(6-(2-(2,2,2-trifluoroacetyl)hydrazine-1-carbonyl)pyrazin-2-yl)-2,6-diazaspiro[3.4]octane-2-carboxylate (500 mg, 90%) as a yellow oil. MS m/z: 445 [M+H]⁺

Step 4: tert-butyl 6-(6-(5-(trifluoromethyl)-1,3,4-thiadiazol-2-yl)pyrazin-2-yl)-2,6-diazaspiro[3.4]octane-2-carboxylate: Followed the general procedure T using tert-butyl 6-(6-(2-(2,2,2-trifluoroacetyl)hydrazine-1-carbonyl)pyrazin-2-yl)-2,6-diazaspiro[3.4]octane-2-carboxylate (500 mg, 1.13 mmol, 1.0 equiv.) as the starting material to give tert-butyl 6-(6-(5-(trifluoromethyl)-1,3,4-thiadiazol-2-yl)pyrazin-2-yl)-2,6-diazaspiro[3.4]octane-2-carboxylate (90 mg, 18%) as a yellow green solid. MS m/z: 443 [M+H]⁺.

Step 5: 2-(6-(2,6-diazaspiro[3.4]octan-6-yl)pyrazin-2-yl)-5-(trifluoromethyl)-1,3,4-thiadiazole: Followed the general procedure H using tert-butyl 6-(6-(5-(trifluoromethyl)-1,3,4-thiadiazol-2-yl)pyrazin-2-yl)-2,6-diazaspiro[3.4]octane-2-carboxylate (90 mg) as the starting material to give the crude product 2-(6-(2,6-diazaspiro[3.4]octan-6-yl)pyrazin-2-yl)-5-(trifluoromethyl)-1,3,4-thiadiazole (60 mg). MS m/z: 333 [M+H]⁺.

Step 6: 2-(trifluoromethyl)-5-(6-(2-(2-(trifluoromethyl)pyridin-4-diazaspiro[3.4]octan-6-yl)pyrazin-2-yl)-1,3,4-thiadiazole: Followed the general procedure I using 2-(6-(2,6-diazaspiro[3.4]octan-6-yl)pyrazin-2-yl)-5-(trifluoromethyl)-1,3,4-thiadiazole (60 mg, 0.175 mmol, 1.0 equiv.) and 4-chloro-2-(trifluoromethyl)pyridine (38.3 mg, 0.21 mmol, 1.2 equiv.) as the starting materials to give 2-(trifluoromethyl)-5-(6-(2-(2-(trifluoromethyl)pyridin-4-yl)-2,6-diazaspiro[3.4]octan-6-yl)pyrazin-2-yl)-1,3,4-thiadiazole (31.2 mg, 36%) as a yellow solid. ¹H NMR (400 MHz, DMSO-d₆) δ 8.66 (s, 1H), 8.30-8.23 (m, 2H), 6.77 (d, J=2.3 Hz, 1H), 6.59 (dd, J=5.7, 2.2 Hz, 1H), 4.10-4.00 (m, 4H), 3.83 (s, 2H), 3.63 (t, J=6.9 Hz, 2H), 2.32 (t, J=6.9 Hz, 2H). MS m/z: 488 [M+H]⁺.

### Example 1024: 2-(trifluoromethyl)-5-(6-(2-(2-(trifluoromethyl)pyridin-4-yl)-2,6-diazaspiro[3.4]octan-6-yl)pyrazin-2-yl)-1,3,4-oxadiazole

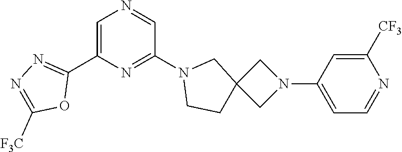 [SMILES: FC(F)(F)c1cc(N2CC3(CCN(c4cncc(-c5nnc(C(F)(F)F)o5)n4)C3)C2)ccn1]

Step 1: tert-butyl 6-(6-(5-(trifluoromethyl)-1,3,4-oxadiazol-2-yl)pyrazin-2-yl)-2,6-diazaspiro[3.4]octane-2-carboxylate. A mixture of tert-butyl 6-(6-(2-(2,2,2-trifluoroacetyl)hydrazine-1-carbonyl)pyrazin-2-yl)-2,6-diazaspiro[3.4]octane-2-carboxylate (500 mg, 1.13 mmol, 1.0 equiv) in THF (9.00 mL) was added Lawesson's Reagent (1.34 g, 5.62 mmol, 5.0 equiv) under N₂ atmosphere. The resulting mixture was stirred for overnight at 70° C. under nitrogen atmosphere. Desired product could be detected by LCMS. The residue was purified by silica gel column chromatography, eluted with DCM:MeOH=5/1 to afford tert-butyl 6-(6-(5-(trifluoromethyl)-1,3,4-oxadiazol-2-yl)pyrazin-2-yl)-2,6-diazaspiro[3.4]octane-2-carboxylate (170 mg, 35%) as a yellow solid. MS m/z: 427 [M+H]⁺.

Step 2: 2-(6-(2,6-diazaspiro[3.4]octan-6-yl)pyrazin-2-yl)-5-(trifluoromethyl)-1,3,4-oxadiazole: Followed the general procedure H using tert-butyl 6-(6-(5-(trifluoromethyl)-1,3,4-oxadiazol-2-yl)pyrazin-2-yl)-2,6-diazaspiro[3.4]octane-2-carboxylate (170 mg) as the starting material to give the crude product 2-(6-(2,6-diazaspiro[3.4]octan-6-yl)pyrazin-2-yl)-5-(trifluoromethyl)-1,3,4-oxadiazole (100 mg). MS m/z: 327 [M+H]⁺.

Step 3: 2-(trifluoromethyl)-5-(6-(2-(2-(trifluoromethyl)pyridin-4-yl)-2,6-diazaspiro[3.4]octan-6-yl)pyrazin-2-yl)-1,3,4-oxadiazole: Followed the general procedure I using 2-(6-(2,6-diazaspiro[3.4]octan-6-yl)pyrazin-2-yl)-5-(trifluoromethyl)-1,3,4-oxadiazole (50 mg, 0.153 mmol, 1.0 equiv.) and 4-chloro-2-(trifluoromethyl)pyridine (33.4 mg, 0.183 mmol, 1.2 equiv.) as the starting materials to give 2-(trifluoromethyl)-5-(6-(2-(2-(trifluoromethyl)pyridin-4-yl)-2,6-diazaspiro[3.4]octan-6-yl)pyrazin-2-yl)-1,3,4-oxadiazole (10 mg, 13%) as a yellow green solid. ¹H NMR (400 MHz, DMSO-d₆) δ 8.58 (s, 1H), 8.29 (s, 1H), 8.25 (d, J=5.6 Hz, 1H), 6.75 (d, J=2.3 Hz, 1H), 6.58 (dd, J=5.8, 2.2 Hz, 1H), 4.08 (d, J=8.4 Hz, 2H), 4.02 (d, J=8.3 Hz, 2H), 3.83 (s, 2H), 3.64 (s, 2H), 2.32 (t, J=6.8 Hz, 2H). MS m/z: 472 [M+H]⁺.

### Example 1025: 2-(trifluoromethyl)-5-(6-(2-(2-(trifluoromethyl)pyrimidin-5-yl)-2,6-diazaspiro[3.4]octan-6-yl)pyrazin-2-yl)-1,3,4-thiadiazole

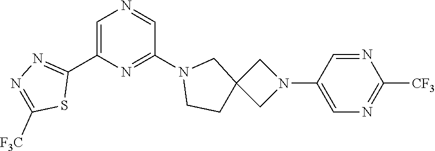 [SMILES: FC(F)(F)c1ncc(N2CC3(CCN(c4cncc(-c5nnc(C(F)(F)F)s5)n4)C3)C2)cn1]

Step 1: tert-butyl 6-(6-(methoxycarbonyl)pyrazin-2-yl)-2,6-diazaspiro[3.4]octane-2-carboxylate: Followed the general procedure I using 6-chloropyrazine-2-carboxylate (1 g, 5.78 mmol, 1.0 equiv.) and tert-butyl 2,6-diazaspiro[3.4]octane-2-carboxylate (1 g, 5.78 mmol, 1.0 equiv.) as the starting materials to give tert-butyl 6-(6-(methoxycarbonyl)pyrazin-2-yl)-2,6-diazaspiro[3.4]octane-2-carboxylate (1 g, 49%) as a white solid. MS m/z: 349 [M+H]⁺.

Step 2: tert-butyl 6-(6-(hydrazinecarbonyl)pyrazin-2-yl)-2,6-diazaspiro[3.4]octane-2-carboxylate: Followed the general procedure S using tert-butyl 6-(6-(methoxycarbonyl)pyrazin-2-yl)-2,6-diazaspiro[3.4]octane-2-carboxylate (1 g, 2.88 mmol, 1.0 equiv.) as the starting material to give tert-butyl 6-(6-(hydrazinecarbonyl)pyrazin-2-yl)-2,6-diazaspiro[3.4]octane-2-carboxylate (900 mg, 90%) as a white solid. MS m/z: 349 [M+H]⁺

Step 3: tert-butyl 6-(6-(2-(2,2,2-trifluoroacetyl)hydrazine-1-carbonyl)pyrazin-2-yl)-2,6-diazaspiro[3.4]octane-2-carboxylate: Followed the general procedure AF using tert-butyl 6-(6-(hydrazinecarbonyl)pyrazin-2-yl)-2,6-diazaspiro[3.4]octane-2-carboxylate (900 mg, 2.58 mmol, 1.0 equiv.) as the starting material to give tert-butyl 6-(6-(2-(2,2,2-trifluoroacetyl)hydrazine-1-carbonyl)pyrazin-2-yl)-2,6-diazaspiro[3.4]octane-2-carboxylate (1 g, 90%) as a yellow oil. MS m/z: 445 [M+H]⁺

Step 4: tert-butyl 6-(6-(5-(trifluoromethyl)-1,3,4-oxadiazol-2-yl)pyrazin-2-yl)-2,6-diazaspiro[3.4]octane-2-carboxylate: Followed the general procedure T using tert-butyl 6-(6-(2-(2,2,2-trifluoroacetyl)hydrazine-1-carbonyl)pyrazin-2-yl)-2,6-diazaspiro[3.4]octane-2-carboxylate (500 mg, 1.13 mmol, 1.0 equiv.) as the starting material to give tert-butyl 6-(6-(5-(trifluoromethyl)-1,3,4-thiadiazol-2-yl)pyrazin-2-yl)-2,6-diazaspiro[3.4]octane-2-carboxylate (160 mg, 32%) as a yellow green solid. MS m/z: 443 [M+H]⁺.

Step 5: 2-(6-(2,6-diazaspiro[3.4]octan-6-yl)pyrazin-2-yl)-5-(trifluoromethyl)-1,3,4-oxadiazole: Followed the general procedure H using tert-butyl 6-(6-(5-(trifluoromethyl)-1,3,4-thiadiazol-2-yl)pyrazin-2-yl)-2,6-diazaspiro[3.4]octane-2-carboxylate (160 mg) as the starting material to give the crude product 2-(6-(2,6-diazaspiro[3.4]octan-6-yl)pyrazin-2-yl)-5-(trifluoromethyl)-1,3,4-thiadiazole (100 mg). MS m/z: 333 [M+H]⁺.

Step 6: 2-(trifluoromethyl)-5-(6-(2-(2-(trifluoromethyl)pyrimidin-5-yl)-2,6-diazaspiro[3.4]octan-6-yl)pyrazin-2-yl)-1,3,4-thiadiazole: Followed the general procedure Y using 2-(6-(2,6-diazaspiro[3.4]octan-6-yl)pyrazin-2-yl)-5-(trifluoromethyl)-1,3,4-oxadiazole (80 mg, 0.245 mmol, 1.0 equiv.) and 5-bromo-2-(trifluoromethyl)pyrimidine (66.7 mg, 0.294 mmol, 1.2 equiv.) as the starting materials to give 2-(trifluoromethyl)-5-(6-(2-(2-(trifluoromethyl)pyrimidin-5-yl)-2,6-diazaspiro[3.4]octan-6-yl)pyrazin-2-yl)-1,3,4-thiadiazole (65.1 mg, 57%) as a yellow solid. ¹H NMR (400 MHz, DMSO-d₆) δ 8.67 (s, 1H), 8.28 (s, 1H), 8.19 (s, 2H), 4.16-4.06 (m, 4H), 3.84 (s, 2H), 3.63 (t, J=6.9 Hz, 2H), 2.34 (t, J=7.0 Hz, 2H). MS m/z: 489 [M+H]⁺.

### Example 1026: 2-(trifluoromethyl)-5-(6-(2-(2-(trifluoromethyl)pyrimidin-5-yl)-2,6-diazaspiro[3.4]octan-6-yl)pyrazin-2-yl)-1,3,4-oxadiazole

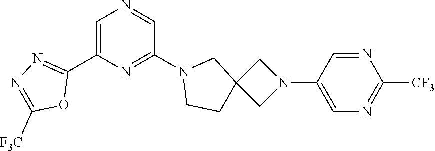 [SMILES: FC(F)(F)c1ncc(N2CC3(CCN(c4cncc(-c5nnc(C(F)(F)F)o5)n4)C3)C2)cn1]

Step 1: 2-(trifluoromethyl)-5-(6-(2-(2-(trifluoromethyl)pyrimidin-5-yl)-2,6-diazaspiro[3.4]octan-6-yl)pyrazin-2-yl)-1,3,4-oxadiazole: Followed the general procedure Y using 2-(6-(2,6-diazaspiro[3.4]octan-6-yl)pyrazin-2-yl)-5-(trifluoromethyl)-1,3,4-oxadiazole (80 mg, 0.245 mmol, 1.0 equiv.) and 5-bromo-2-(trifluoromethyl)pyrimidine (66.7 mg, 0.294 mmol, 1.2 equiv.) as the starting materials to give 2-(trifluoromethyl)-5-(6-(2-(2-(trifluoromethyl)pyrimidin-5-yl)-2,6-diazaspiro[3.4]octan-6-yl)pyrazin-2-yl)-1,3,4-oxadiazole (15.3 mg, 13%) as a off-white solid. ¹H NMR (400 MHz, DMSO-d₆) δ 8.58 (s, 1H), 8.29 (s, 1H), 8.18 (s, 2H), 4.14 (d, J=8.2 Hz, 2H), 4.09 (d, J=8.1 Hz, 2H), 3.84 (s, 2H), 3.65 (t, J=6.9 Hz, 2H), 2.34 (t, J=6.9 Hz, 2H). MS m/z: 473.05 [M+H]⁺.

### Example 1027: 2-(6-(2-(2-(trifluoromethyl)pyridin-4-yl)-2,6-diazaspiro[3.4]octan-6-yl)pyrazin-2-yl)thiazole

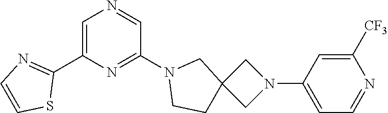 [SMILES: FC(F)(F)c1cc(N2CC3(CCN(c4cncc(-c5nccs5)n4)C3)C2)ccn1]

Step 1: 6-(6-bromopyrazin-2-yl)-2-(2-(trifluoromethyl)pyridin-4-yl)-2,6-diazaspiro[3.4]octane: Followed the general procedure I using 2-(2-(trifluoromethyl)pyridin-4-yl)-2,6-diazaspiro[3.4]octane (400 mg, 1.36 mmol, 1.00 equiv.) and 2,6-dibromopyrazine (388 mg, 1.63 mmol, 1.20 equiv.) as the starting materials to give 6-(6-bromopyrazin-2-yl)-2-(2-(trifluoromethyl)pyridin-4-yl)-2,6-diazaspiro[3.4]octane (400 mg, 70.9%) as a white solid. MS m/z: 414 [M+H]⁺.

Step 2: 2-(6-(2-(2-(trifluoromethyl)pyridin-4-yl)-2,6-diazaspiro[3.4]octan-6-yl)pyrazin-2-yl)thiazole: Followed the general procedure X using 6-(6-bromopyrazin-2-yl)-2-(2-(trifluoromethyl)pyridin-4-yl)-2,6-diazaspiro[3.4]octane (150 mg, 0.362 mmol, 1.00 equiv.) and 2-(tributylstannyl)thiazole (203 mg, 0.543 mmol, 1.50 equiv.) as the starting materials, Pd(PPh₃)₂Cl₂ ((25.4 mg, 0.036 mmol, 0.100 equiv.) as the catalyst to give 2-(6-(2-(2-(trifluoromethyl)pyridin-4-yl)-2,6-diazaspiro[3.4]octan-6-yl)pyrazin-2-yl)thiazole (39.8 mg, 26.1%) as a yellow solid. ¹H NMR (400 MHz, Chloroform-d) δ 8.72 (s, 1H), 8.33 (d, J=5.6 Hz, 1H), 7.98-7.88 (m, 2H), 7.45 (d, J=2.4 Hz, 1H), 6.64 (dd, J=5.6 Hz, 2.4 Hz, 1H), 6.44-6.32 (m, 1H), 4.19-3.90 (m, 4H), 3.87 (s, 2H), 3.71 (t, 2H), 2.40 (t, 2H). MS m/z: 419.10 [M+H]⁺.

### Example 1028: 5-(trifluoromethyl)-2-(6-(2-(2-(trifluoromethyl)pyridin-4-yl)-2,6-diazaspiro[3.4]octan-6-yl)pyrazin-2-yl)thiazole

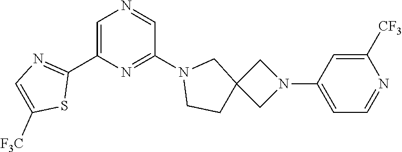 [SMILES: FC(F)(F)c1cc(N2CC3(CCN(c4cncc(-c5ncc(C(F)(F)F)s5)n4)C3)C2)ccn1]

Step 1: 6-(6-(tributylstannyl)pyrazin-2-yl)-2-(2-(trifluoromethyl)pyridin-4-yl)-2,6-diazaspiro[3.4]octane: Followed the general procedure X using 6-(6-bromopyrazin-2-yl)-2-(2-(trifluoromethyl)pyridin-4-yl)-2,6-diazaspiro[3.4]octane (200 mg, 0.48 mmol, 1.0 equiv.) and Sn₂nBu₆ (560 mg, 0.96 mmol, 2.0 equiv.) as the starting materials to give 6-(6-(tributylstannyl)pyrazin-2-yl)-2-(2-(trifluoromethyl)pyridin-4-yl)-2,6-diazaspiro[3.4]octane (120 mg, 40%) as a colorless oil. MS m/z: 626 [M+H]⁺.

Step 2: 5-(trifluoromethyl)-2-(6-(2-(2-(trifluoromethyl)pyridin-4-yl)-2,6-diazaspiro[3.4]octan-6-yl)pyrazin-2-yl)thiazole: Followed the general procedure X using 6-(6-(tributylstannyl)pyrazin-2-yl)-2-(2-(trifluoromethyl)pyridin-4-yl)-2,6-diazaspiro[3.4]octane (120 mg, 0.192 mmol, 1.0 equiv.) and 2-bromo-5-(trifluoromethyl)thiazole (50 mg, 0.21 mmol, 1.1 equiv.) as the starting materials to give 5-(trifluoromethyl)-2-(6-(2-(2-(trifluoromethyl)pyridin-4-yl)-2,6-diazaspiro[3.4]octan-6-yl)pyrazin-2-yl)thiazole (19.4 mg, 21%) as a white solid. ¹H NMR (300 MHz, DMSO-d₆) δ 8.60 (q, J=1.3 Hz, 1H), 8.53 (s, 1H), 8.26 (d, J=5.6 Hz, 1H), 8.21 (s, 1H), 6.76 (d, J=2.2 Hz, 1H), 6.59 (dd, J=5.7, 2.2 Hz, 1H), 4.05 (q, J=8.4 Hz, 4H), 3.82 (s, 2H), 3.63 (t, J=6.8 Hz, 2H), 2.32 (t, J=6.8 Hz, 2H). MS m/z: 487.15 [M+H]⁺.

### Example 1029: 5-(6-(2-(2-(trifluoromethyl)pyridin-4-yl)-2,6-diazaspiro[3.4]octan-6-yl)pyrazin-2-yl)thiazole

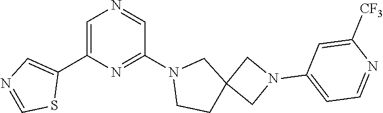 [SMILES: FC(F)(F)c1cc(N2CC3(CCN(c4cncc(-c5cncs5)n4)C3)C2)ccn1]

Step 1: 5-(4,4,5,5-tetramethyl-1,3,2-dioxaborolan-2-yl)thiazole: Followed the general procedure L using 5-bromothiazole (50 mg, 0.305 mmol, 1.0 equiv.) and 4,4,4′,4′,5,5,5′,5′-octamethyl-2,2′-bi(1,3,2-dioxaborolane) (232 mg, 0.915 mmol, 3.0 equiv.) as the starting materials to give 5-(4,4,5,5-tetramethyl-1,3,2-dioxaborolan-2-yl)thiazole (50 mg, 77%) as a white solid. MS m/z: 212 [M+H]⁺.

Step 2: 5-(6-(2-(2-(trifluoromethyl)pyridin-4-yl)-2,6-diazaspiro[3.4]octan-6-yl)pyrazin-2-yl)thiazole: Followed the general procedure L using 5-(4,4,5,5-tetramethyl-1,3,2-dioxaborolan-2-yl)thiazole (50 mg, 0.237 mmol, 1.0 equiv.) and 6-(6-bromopyrazin-2-yl)-2-(2-(trifluoromethyl)pyridin-4-yl)-2,6-diazaspiro[3.4]octane (98.1 mg, 0.237 mmol, 1.0 equiv.) as the starting materials to give 5-(6-(2-(2-(trifluoromethyl)pyridin-4-yl)-2,6-diazaspiro[3.4]octan-6-yl)pyrazin-2-yl)thiazole (53 mg, 53%) as a yellow solid. ¹H NMR (300 MHz, DMSO-d₆) δ 9.17 (d, J=0.6 Hz, 1H), 8.61 (d, J=0.7 Hz, 1H), 8.43 (s, 1H), 8.25 (d, J=5.7 Hz, 1H), 7.93 (s, 1H), 6.76 (d, J=2.2 Hz, 1H), 6.58 (dd, J=5.6, 2.3 Hz, 1H), 4.04 (q, J=8.4 Hz, 4H), 3.77 (s, 2H), 3.58 (t, J=6.9 Hz, 2H), 2.29 (t, J=6.8 Hz, 2H). MS m/z: 419 [M+H]⁺.

### Example 1030: 1-(2,2-difluoroethyl)-6-(2-(5-isopropyl-2-(trifluoromethyl)pyridin-4-yl)-2,6-diazaspiro[3.4]octan-6-yl)-1H-pyrazolo[3,4-b]pyrazine

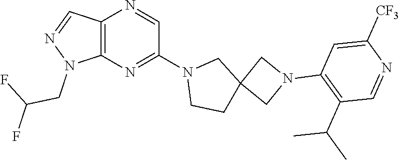 [SMILES: CC(C)c1cnc(C(F)(F)F)cc1N1CC2(CCN(c3cnc4cnn(CC(F)F)c4n3)C2)C1]

Step 1: 1-(2,2-difluoroethyl)-6-(2-(5-(prop-1-en-2-yl)-2-(trifluoromethyl)pyridin-4-yl)-2,6-diazaspiro[3.4]octan-6-yl)-1H-pyrazolo[3,4-b]pyrazine: Followed the general procedure L using 6-(2-(5-bromo-2-(trifluoromethyl)pyridin-4-yl)-2,6-diazaspiro[3.4]octan-6-yl)-1-(2,2-difluoroethyl)-1H-pyrazolo[3,4-b]pyrazine (150 mg, 0.289 mmol, 1.0 equiv.) and 4,4,5,5-tetramethyl-2-(prop-1-en-2-yl)-1,3,2-dioxaborolane (97.3 mg, 0.578 mmol, 2.0 equiv.) as the starting materials to give 1-(2,2-difluoroethyl)-6-(2-(5-(prop-1-en-2-yl)-2-(trifluoromethyl)pyridin-4-yl)-2,6-diazaspiro[3.4]octan-6-yl)-1H-pyrazolo[3,4-b]pyrazine (120 mg, 86%) as a white solid. MS m/z: 480 [M+H]⁺.

Step 2: 1-(2,2-difluoroethyl)-6-(2-(5-isopropyl-2-(trifluoromethyl)pyridin-4-yl)-2,6-diazaspiro[3.4]octan-6-yl)-1H-pyrazolo[3,4-b]pyrazine: Followed the general procedure U using 1-(2,2-difluoroethyl)-6-(2-(5-(prop-1-en-2-yl)-2-(trifluoromethyl)pyridin-4-yl)-2,6-diazaspiro[3.4]octan-6-yl)-1H-pyrazolo[3,4-b]pyrazine (100 mg, 0.209 mmol, 1.0 equiv.) as the starting material to give 1-(2,2-difluoroethyl)-6-(2-(5-isopropyl-2-(trifluoromethyl)pyridin-4-yl)-2,6-diazaspiro[3.4]octan-6-yl)-1H-pyrazolo[3,4-b]pyrazine (26.8 mg, 25%) as a white solid. ¹H NMR (400 MHz, DMSO-d₆) δ 8.25 (s, 1H), 8.15-8.00 (m, 2H), 6.80-6.17 (m, 2H), 4.68 (td, 2H), 4.25-4.00 (m, 4H), 3.87 (s, 2H), 3.68 (t, J=6.9 Hz, 2H), 3.18-2.99 (m, 1H), 2.31 (t, J=6.9 Hz, 2H), 1.24 (d, J=6.8 Hz, 6H). MS m/z: 482.10 [M+H]⁺.

### Example 1031: 1-(2,2-difluoroethyl)-6-(2-(5-propyl-2-(trifluoromethyl)pyridin-4-yl)-2,6-diazaspiro[3.4]octan-6-yl)-1H-pyrazolo[3,4-b]pyrazine

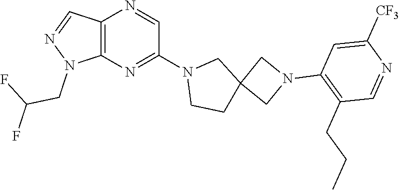 [SMILES: CCCc1cnc(C(F)(F)F)cc1N1CC2(CCN(c3cnc4cnn(CC(F)F)c4n3)C2)C1]

Step 1: (E)-1-(2,2-difluoroethyl)-6-(2-(5-(prop-1-en-1-yl)-2-(trifluoromethyl)pyridin-4-yl)-2,6-diazaspiro[3.4]octan-6-yl)-1H-pyrazolo[3,4-b]pyrazine: Followed the general procedure L using 6-(2-(5-bromo-2-(trifluoromethyl)pyridin-4-yl)-2,6-diazaspiro[3.4]octan-6-yl)-1-(2,2-difluoroethyl)-1H-pyrazolo[3,4-b]pyrazine (150 mg, 0.289 mmol, 1.0 equiv.) and (E)-4,4,5,5-tetramethyl-2-(prop-1-en-1-yl)-1,3,2-dioxaborolane (97.3 mg, 0.578 mmol, 2.0 equiv.) as the starting materials to give (E)-1-(2,2-difluoroethyl)-6-(2-(5-(prop-1-en-1-yl)-2-(trifluoromethyl)pyridin-4-yl)-2,6-diazaspiro[3.4]octan-6-yl)-1H-pyrazolo[3,4-b]pyrazine (140 mg, 85%) as a white solid. MS m/z: 480 [M+H]⁺.

Step 2: 1-(2,2-difluoroethyl)-6-(2-(5-propyl-2-(trifluoromethyl)pyridin-4-yl)-2,6-diazaspiro[3.4]octan-6-yl)-1H-pyrazolo[3,4-b]pyrazine: Followed the general procedure U using (E)-1-(2,2-difluoroethyl)-6-(2-(5-(prop-1-en-1-yl)-2-(trifluoromethyl)pyridin-4-yl)-2,6-diazaspiro[3.4]octan-6-yl)-1H-pyrazolo[3,4-b]pyrazine (100 mg, 0.217 mmol, 1.0 equiv.) as the starting material to give 1-(2,2-difluoroethyl)-6-(2-(5-propyl-2-(trifluoromethyl)pyridin-4-yl)-2,6-diazaspiro[3.4]octan-6-yl)-1H-pyrazolo[3,4-b]pyrazine (22.1 mg, 21%) as a white solid. ¹H NMR (300 MHz, DMSO-d₆) δ 8.23-7.96 (m, 3H), 6.80-6.15 (m, 2H), 4.73 (td, 2H), 4.35-4.10 (m, 4H), 3.87 (s, 2H), 3.69 (t, J=6.7 Hz, 2H), 2.63-2.53 (m, 2H), 2.32 (t, J=7.1 Hz, 2H), 1.55 (q, J=7.5 Hz, 2H), 0.92 (t, J=7.3 Hz, 3H). MS m/z: 482.10 [M+H]⁺.

### Example 1032: 1-(2,2-difluoroethyl)-6-(2-(5-ethyl-2-(trifluoromethyl)pyridin-4-yl)-2,6-diazaspiro[3.4]octan-6-yl)-1H-pyrazolo[3,4-b]pyrazine

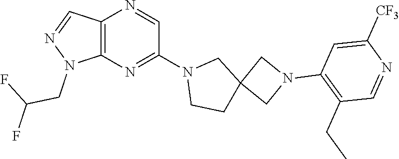 [SMILES: CCc1cnc(C(F)(F)F)cc1N1CC2(CCN(c3cnc4cnn(CC(F)F)c4n3)C2)C1]

Step 1:1-(2,2-difluoroethyl)-6-(2-(2-(trifluoromethyl)-5-vinylpyridin-4-yl)-2,6-diazaspiro[3.4]octan-6-yl)-1H-pyrazolo[3,4-b]pyrazine: Followed the general procedure L using 6-(2-(5-bromo-2-(trifluoromethyl)pyridin-4-yl)-2,6-diazaspiro[3.4]octan-6-yl)-1-(2,2-difluoroethyl)-1H-pyrazolo[3,4-b]pyrazine (150 mg, 0.289 mmol, 1.0 equiv.) and 4,4,5,5-tetramethyl-2-vinyl-1,3,2-dioxaborolane (72.9 mg, 0.578 mmol, 2.0 equiv.) as the starting materials to give 1-(2,2-difluoroethyl)-6-(2-(2-(trifluoromethyl)-5-vinylpyridin-4-yl)-2,6-diazaspiro[3.4]octan-6-yl)-1H-pyrazolo[3,4-b]pyrazine (100 mg, 74%) as a light yellow solid. MS m/z: 466 [M+H]⁺.

Step 2: 1-(2,2-difluoroethyl)-6-(2-(5-ethyl-2-(trifluoromethyl)pyridin-4-yl)-2,6-diazaspiro[3.4]octan-6-yl)-1H-pyrazolo[3,4-b]pyrazine: Followed the general procedure U using 1-(2,2-difluoroethyl)-6-(2-(2-(trifluoromethyl)-5-vinylpyridin-4-yl)-2,6-diazaspiro[3.4]octan-6-yl)-1H-pyrazolo[3,4-b]pyrazine (100 mg, 0.215 mmol, 1.0 equiv) as the starting material to give 1-(2,2-difluoroethyl)-6-(2-(5-ethyl-2-(trifluoromethyl)pyridin-4-yl)-2,6-diazaspiro[3.4]octan-6-yl)-1H-pyrazolo[3,4-b]pyrazine (40.4 mg, 39%) as a white solid. ¹H NMR (400 MHz, DMSO-d₆) δ 8.19-8.10 (m, 3H), 6.64-6.22 (m, 2H), 4.69 (td, 2H), 4.25-4.10 (m, 4H), 3.86 (s, 2H), 3.67 (d, J=7.0 Hz, 2H), 2.61 (q, J=7.4 Hz, 2H), 2.31 (t, J=6.5 Hz, 2H), 1.15 (t, J=7.4 Hz, 3H). MS m/z: 468.15 [M+H]⁺.

### Example 1033: 6-(2-(2-(trifluoromethyl)pyridin-4-yl)-2,6-diazaspiro[3.4]octan-6-yl)-1-(3,3,3-trifluoropropyl)-1H-pyrazolo[3,4-b]pyrazine

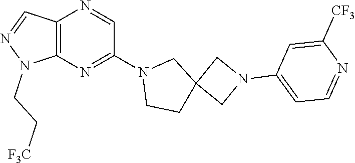 [SMILES: FC(F)(F)CCn1ncc2ncc(N3CCC4(CN(c5ccnc(C(F)(F)F)c5)C4)C3)nc21]

Step 1: 6-chloro-1-(3,3,3-trifluoropropyl)-1H-pyrazolo[3,4-b]pyrazine: Followed the general procedure K using 6-chloro-1H-pyrazolo[3,4-b]pyrazine (400 mg, 2.59 mmol, 1.0 equiv.), and 1,1,1-trifluoro-3-iodopropane (696 mg, 3.11 mmol, 1.2 equiv.) as the starting materials to give 6-chloro-1-(3,3,3-trifluoropropyl)-1H-pyrazolo[3,4-b]pyrazine (250 mg, 39%) as a yellow oil. MS m/z: 251 [M+H]⁺

Step 2: 6-(2-(2-(trifluoromethyl)pyridin-4-yl)-2,6-diazaspiro[3.4]octan-6-yl)-1-(3,3,3-trifluoropropyl)-1H-pyrazolo[3,4-b]pyrazine: Followed the general procedure I using 6-chloro-1-(3,3,3-trifluoropropyl)-1H-pyrazolo[3,4-b]pyrazine (50 mg, 0.2 mmol, 1.0 equiv.) and 2-(2-(trifluoromethyl)pyridin-4-yl)-2,6-diazaspiro[3.4]octane (51.3 mg, 0.2 mmol, 1.0 equiv.) as the starting materials to give 6-(2-(2-(trifluoromethyl)pyridin-4-yl)-2,6-diazaspiro[3.4]octan-6-yl)-1-(3,3,3-trifluoropropyl)-1H-pyrazolo[3,4-b]pyrazine (38 mg, 40%) as a white solid. ¹H NMR (300 MHz, DMSO-d₆) δ 8.26 (d, J=5.7 Hz, 1H), 8.09 (d, J=1.9 Hz, 2H), 6.77 (d, J=2.2 Hz, 1H), 6.65-6.53 (m, 1H), 4.51 (t, J=6.6 Hz, 2H), 4.12-3.99 (m, 4H), 3.86 (s, 2H), 3.68 (t, J=6.9 Hz, 2H), 3.02-2.83 (m, 2H), 2.32 (t, J=6.9 Hz, 2H). MS m/z: 472.2 [M+H]⁺.

### Example 1034: 1-(2-(trifluoromethoxy)ethyl)-6-(2-(2-(trifluoromethyl)pyridin-4-yl)-2,6-diazaspiro[3.4]octan-6-yl)-1H-pyrazolo[3,4-b]pyrazine

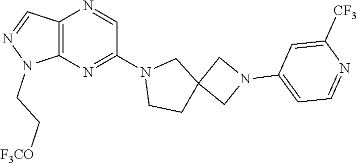 [SMILES: FC(F)(F)OCCn1ncc2ncc(N3CCC4(CN(c5ccnc(C(F)(F)F)c5)C4)C3)nc21]

Step 1: 6-chloro-1-(2-(trifluoromethoxy)ethyl)-1H-pyrazolo[3,4-b]pyrazine: Followed the general procedure I using 1-bromo-2-(trifluoromethoxy)ethane (200 mg, 1.04 mmol, 1.0 equiv.) and 6-chloro-1H-pyrazolo[3,4-b]pyrazine (192 mg, 1.24 mmol, 1.2 equiv.) as the starting materials to give 6-chloro-1-(2-(trifluoromethoxy)ethyl)-1H-pyrazolo[3,4-b]pyrazine (60 mg, 22%) as a white solid. MS m/z: 267 [M+H]⁺.

Step 2: 1-(2-(trifluoromethoxy)ethyl)-6-(2-(2-(trifluoromethyl)pyridin-4-yl)-2,6-diazaspiro[3.4]octan-6-yl)-1H-pyrazolo[3,4-b]pyrazine: Followed the general procedure I using 6-chloro-1-(2-(trifluoromethoxy)ethyl)-1H-pyrazolo[3,4-b]pyrazine (50 mg, 0.188 mmol, 1.0 equiv.) and 2-(2-(trifluoromethyl)pyridin-4-yl)-2,6-diazaspiro[3.4]octane (57.9 mg, 0.226 mmol, 1.2 equiv.) as the starting materials to give 1-(2-(trifluoromethoxy)ethyl)-6-(2-(2-(trifluoromethyl)pyridin-4-yl)-2,6-diazaspiro[3.4]octan-6-yl)-1H-pyrazolo[3,4-b]pyrazine (28.2 mg, 30%) as a white solid. ¹H NMR (300 MHz, DMSO-d₆) δ 8.26 (d, J=5.6 Hz, 1H), 8.10 (d, J=5.3 Hz, 2H), 6.77 (d, J=2.2 Hz, 1H), 6.59 (dd, J=5.7, 2.3 Hz, 1H), 4.56 (s, 4H), 4.12-3.97 (m, 4H), 3.86 (s, 2H), 3.67 (t, J=6.9 Hz, 2H), 2.32 (t, J=6.9 Hz, 2H). MS m/z: 488 [M+H]⁺.

### Example 1035: 1-(2,2-difluoroethyl)-5-methyl-6-(2-(2-(trifluoromethyl)pyridin-4-yl)-2,6-diazaspiro[3.4]octan-6-yl)-1H-pyrazolo[3,4-b]pyrazine

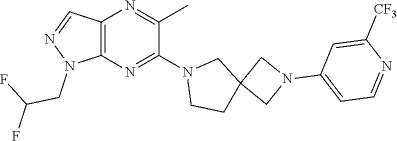 [SMILES: Cc1nc2cnn(CC(F)F)c2nc1N1CCC2(CN(c3ccnc(C(F)(F)F)c3)C2)C1]

Step 1: 1-(2,2-difluoroethyl)-5-methyl-6-(2-(2-(trifluoromethyl)pyridin-4-yl)-2,6-diazaspiro[3.4]octan-6-yl)-1H-pyrazolo[3,4-b]pyrazine: Followed the general procedure D using 1-(2,2-difluoroethyl)-5-methyl-6-(2,6-diazaspiro[3.4]octan-6-yl)-1H-pyrazolo[3,4-b]pyrazine (100 mg, 325 μmol, 1.0 equiv) and 4-bromo-2-(trifluoromethyl)pyridine (73 mg, 324 μmol, 1.0 equiv) as the starting materials to give 1-(2,2-difluoroethyl)-5-methyl-6-(2-(2-(trifluoromethyl)pyrimidin-5-yl)-2,6-diazaspiro[3.4]octan-6-yl)-1H-pyrazolo[3,4-b]pyrazine (18.4 mg, 39%) as a yellow solid. ¹H NMR (400 MHz, DMSO-d₆) δ 8.24 (d, J=5.7 Hz, 1H), 8.04 (s, 1H), 6.75 (d, J=2.2 Hz, 1H), 6.60-6.56 (m, 1H), 6.46-6.29 (m, 1H), 4.68 (td, J=14.9, 3.9 Hz, 2H), 4.06 (d, J=8.3 Hz, 2H), 4.01 (d, J=8.3 Hz, 2H), 3.97 (s, 2H), 3.78 (t, J=6.8 Hz, 2H), 2.69 (s, 3H), 2.23 (t, J=6.8 Hz, 2H). MS m/z: 454.15 [M+H]⁺.

### Example 1036: 1-(2,2-difluoroethyl)-5-methyl-6-(2-(2-(trifluoromethyl)pyrimidin-5-yl)-2,6-diazaspiro[3.4]octan-6-yl)-1H-pyrazolo[3,4-b]pyrazine

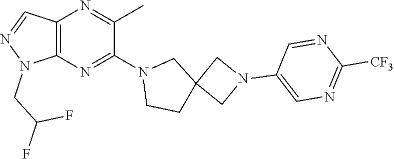 [SMILES: Cc1nc2cnn(CC(F)F)c2nc1N1CCC2(CN(c3cnc(C(F)(F)F)nc3)C2)C1]

Step 1: tert-butyl 6-(1-(2,2-difluoroethyl)-1H-pyrazolo[3,4-b]pyrazin-6-yl)-2,6-diazaspiro[3.4]octane-2-carboxylate: Followed the general procedure I using 6-chloro-1-(2,2-difluoroethyl)-1H-pyrazolo[3,4-b]pyrazine (500 mg, 2.29 mmol, 1.0 equiv.) and tert-butyl 2,6-diazaspiro[3.4]octane-2-carboxylate (533 mg, 2.52 mmol, 1.1 equiv.) as the starting materials to give tert-butyl 6-(1-(2,2-difluoroethyl)-1H-pyrazolo[3,4-b]pyrazin-6-yl)-2,6-diazaspiro[3.4]octane-2-carboxylate (600 mg, 32%) as a yellow solid. MS m/z: 395 [M+H]⁺.

Step 2: tert-butyl 4-(6-oxo-5-((3-(trifluoromethyl)pyrazin-2-yl)methyl)-5,6-dihydropyrido[2,3-b]pyrazin-7-yl)piperazine-1-carboxylate: Followed the general procedure V using tert-butyl 6-(1-(2,2-difluoroethyl)-1H-pyrazolo[3,4-b]pyrazin-6-yl)-2,6-diazaspiro[3.4]octane-2-carboxylate (200 mg, 0.51 mmol, 1.0 equiv.) as the starting materials, NCS (32.4 mg, 0.56 mmol, 1.1 equiv.) as the reagent to give tert-butyl 4-(6-oxo-5-((3-(trifluoromethyl)pyrazin-2-yl)methyl)-5,6-dihydropyrido[2,3-b]pyrazin-7-yl)piperazine-1-carboxylate (170 mg, 50%) as a yellow solid. MS m/z: 429 [M+H]⁺.

Step 3: tert-butyl 6-(1-(2,2-difluoroethyl)-5-methyl-1H-pyrazolo[3,4-b]pyrazin-6-yl)-2,6-diazaspiro[3.4]octane-2-carboxylate: Followed the general procedure L using tert-butyl 4-(6-oxo-5-((3-(trifluoromethyl)pyrazin-2-yl)methyl)-5,6-dihydropyrido[2,3-b]pyrazin-7-yl)piperazine-1-carboxylate (170 mg, 0.40 mmol, 1.0 equiv.) and methylboronic acid (119 mg, 1.98 mmol, 5.0 equiv.) as the starting materials to give the crude product tert-butyl 6-(1-(2,2-difluoroethyl)-5-methyl-1H-pyrazolo[3,4-b]pyrazin-6-yl)-2,6-diazaspiro[3.4]octane-2-carboxylate (100 mg) as a yellow oil. MS m/z: 409 [M+H]⁺.

Step 4: 1-(2,2-difluoroethyl)-5-methyl-6-(2,6-diazaspiro[3.4]octan-6-yl)-1H-pyrazolo[3,4-b]pyrazine: Followed the general procedure H using tert-butyl 6-(1-(2,2-difluoroethyl)-5-methyl-1H-pyrazolo[3,4-b]pyrazin-6-yl)-2,6-diazaspiro[3.4]octane-2-carboxylate (100 mg) as the starting material to give the crude product 1-(2,2-difluoroethyl)-5-methyl-6-(2,6-diazaspiro[3.4]octan-6-yl)-1H-pyrazolo[3,4-b]pyrazine (100 mg). MS m/z: 309 [M+H]⁺.

Step 5: 1-(2,2-difluoroethyl)-5-methyl-6-(2-(2-(trifluoromethyl)pyrimidin-5-yl)-2,6-diazaspiro[3.4]octan-6-yl)-1H-pyrazolo[3,4-b]pyrazine: Followed the general procedure I using 1-(2,2-difluoroethyl)-5-methyl-6-(2,6-diazaspiro[3.4]octan-6-yl)-1H-pyrazolo[3,4-b]pyrazine (100 mg, 0.33 mmol, 1.0 equiv) and 5-bromo-2-(trifluoromethyl)pyrimidine (73.3 mg, 0.32 mmol, 1.0 equiv) as the starting materials to give 1-(2,2-difluoroethyl)-5-methyl-6-(2-(2-(trifluoromethyl)pyrimidin-5-yl)-2,6-diazaspiro[3.4]octan-6-yl)-1H-pyrazolo[3,4-b]pyrazine (18.4 mg, 39%) as a yellow solid. ¹H NMR (400 MHz, Methanol-d₄) δ 8.12 (s, 2H), 7.95 (s, 1H), 6.47-6.15 (m, 1H), 4.73-4.62 (m, 2H), 4.21-4.11 (m, 4H), 4.05 (s, 2H), 3.91 (t, J=6.8 Hz, 2H), 2.77 (s, 3H), 2.36 (t, J=6.8 Hz, 2H). MS m/z: 455.15 [M+H]⁺.

### Example 1037: 2-(5-methyl-6-(2-(2-(trifluoromethyl)pyridin-4-yl)-2,6-diazaspiro[3.4]octan-6-yl)pyrazin-2-yl)-1,3,4-thiadiazole

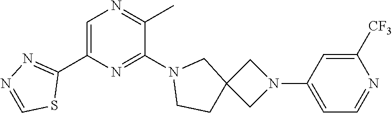 [SMILES: Cc1ncc(-c2nncs2)nc1N1CCC2(CN(c3ccnc(C(F)(F)F)c3)C2)C1]

Step 1: tert-butyl 6-(6-chloro-3-methylpyrazin-2-yl)-2,6-diazaspiro[3.4]octane-2-carboxylate: Followed the general procedure I using 3,5-dichloro-2-methylpyrazine (1 g, 6.14 mmol, 1.0 equiv.) and tert-butyl 2,6-diazaspiro[3.4]octane-2-carboxylate (1.57 g, 7.36 mmol, 1.2 equiv.) as the starting materials to give tert-butyl 6-(6-chloro-3-methylpyrazin-2-yl)-2,6-diazaspiro[3.4]octane-2-carboxylate (1.4 g, 67%) as a white solid. MS m/z: 339 [M+H]⁺.

Step 2: tert-butyl 6-(6-(methoxycarbonyl)-3-methylpyrazin-2-yl)-2,6-diazaspiro[3.4]octane-2-carboxylate: Followed the general procedure N using tert-butyl 6-(6-chloro-3-methylpyrazin-2-yl)-2,6-diazaspiro[3.4]octane-2-carboxylate (1.4 g, 4.13 mmol, 1.0 equiv.) as the starting material to give tert-butyl 6-(6-(methoxycarbonyl)-3-methylpyrazin-2-yl)-2,6-diazaspiro[3.4]octane-2-carboxylate (600 mg, 40%) as a white solid. MS m/z: 363 [M+H]⁺.

Step 3: tert-butyl 6-(6-(hydrazinecarbonyl)-3-methylpyrazin-2-yl)-2,6-diazaspiro[3.4]octane-2-carboxylate: Followed the general procedure S using tert-butyl 6-(6-(methoxycarbonyl)-3-methylpyrazin-2-yl)-2,6-diazaspiro[3.4]octane-2-carboxylate (600 mg, 1.66 mmol, 1.0 equiv.) as the starting material to give tert-butyl 6-(6-(hydrazinecarbonyl)-3-methylpyrazin-2-yl)-2,6-diazaspiro[3.4]octane-2-carboxylate (540 mg, 90%) as a white oil. MS m/z: 363 [M+H]⁺

Step 4: tert-butyl 6-(6-(2-formylhydrazine-1-carbonyl)-3-methylpyrazin-2-yl)-2,6-diazaspiro[3.4]octane-2-carboxylate: Followed the general procedure AF using tert-butyl 6-(6-(hydrazinecarbonyl)-5-methoxypyrazin-2-yl)-2,6-diazaspiro[3.4]octane-2-carboxylate (540 mg, 1.49 mmol, 1.0 equiv.) as the starting material to give tert-butyl 6-(6-(2-formylhydrazine-1-carbonyl)-3-methylpyrazin-2-yl)-2,6-diazaspiro[3.4]octane-2-carboxylate (350 mg, 60%) as a yellow solid. MS m/z: 391 [M+H]⁺

Step 5: tert-butyl 6-(3-methyl-6-(1,3,4-thiadiazol-2-yl)pyrazin-2-yl)-2,6-diazaspiro[3.4]octane-2-carboxylate: Followed the general procedure T using tert-butyl 6-(6-(2-formylhydrazine-1-carbonyl)-3-methylpyrazin-2-yl)-2,6-diazaspiro[3.4]octane-2-carboxylate (350 mg, 0.895 mmol, 1.0 equiv.) as the starting material to give tert-butyl 6-(3-methyl-6-(1,3,4-thiadiazol-2-yl)pyrazin-2-yl)-2,6-diazaspiro[3.4]octane-2-carboxylate (180 mg, 51%) as a yellow green solid. MS m/z: 389 [M+H]⁺.

Step 6: 2-(5-methyl-6-(2,6-diazaspiro[3.4]octan-6-yl)pyrazin-2-yl)-1,3,4-thiadiazole: Followed the general procedure H using tert-butyl 6-(3-methyl-6-(1,3,4-thiadiazol-2-yl)pyrazin-2-yl)-2,6-diazaspiro[3.4]octane-2-carboxylate (180 mg) as the starting material to give the crude product 2-(3-methoxy-6-(2,6-diazaspiro[3.4]octan-6-yl)pyrazin-2-yl)-1,3,4-thiadiazole (120 mg). MS m/z: 289 [M+H]⁺.

Step 7: 2-(5-methyl-6-(2-(2-(trifluoromethyl)pyridin-4-yl)-2,6-diazaspiro[3.4]octan-6-yl)pyrazin-2-yl)-1,3,4-thiadiazole: Followed the general procedure I using 2-(3-methoxy-6-(2,6-diazaspiro[3.4]octan-6-yl)pyrazin-2-yl)-1,3,4-thiadiazole (60 mg, 0.208 mmol, 1.0 equiv.) and 4-chloro-2-(trifluoromethyl)pyridine (45.4 mg, 0.250 mmol, 1.2 equiv.) as the starting materials to give 2-(5-methyl-6-(2-(2-(trifluoromethyl)pyridin-4-yl)-2,6-diazaspiro[3.4]octan-6-yl)pyrazin-2-yl)-1,3,4-thiadiazole (27.1 mg, 30%) as a yellow solid. ¹H NMR (400 MHz, DMSO-d₆) δ 9.68 (s, 1H), 8.57 (s, 1H), 8.25 (d, J=5.6 Hz, 1H), 6.75 (d, J=2.3 Hz, 1H), 6.57 (dd, J=5.7, 2.3 Hz, 1H), 4.08-3.99 (m, 4H), 3.92 (s, 2H), 3.72 (t, J=6.8 Hz, 2H), 2.66 (s, 3H), 2.24 (t, J=6.8 Hz, 2H). MS m/z: 434.05 [M+H]⁺.

### Example 1038: 2-(5-methyl-6-(2-(2-(trifluoromethyl)pyrimidin-5-yl)-2,6-diazaspiro[3.4]octan-6-yl)pyrazin-2-yl)-1,3,4-thiadiazole

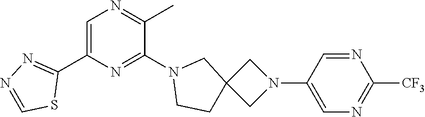 [SMILES: Cc1ncc(-c2nncs2)nc1N1CCC2(CN(c3cnc(C(F)(F)F)nc3)C2)C1]

Step 1: 2-(5-methyl-6-(2-(2-(trifluoromethyl)pyrimidin-5-yl)-2,6-diazaspiro[3.4]octan-6-yl)pyrazin-2-yl)-1,3,4-thiadiazole: Followed the general procedure Y using 2-(5-methyl-6-(2,6-diazaspiro[3.4]octan-6-yl)pyrazin-2-yl)-1,3,4-thiadiazole (60 mg, 0.208 mmol, 1.0 equiv.) and 5-bromo-2-(trifluoromethyl)pyrimidine (56.6 mg, 0.25 mmol, 1.2 equiv.) as the starting materials to give 2-(5-methyl-6-(2-(2-(trifluoromethyl)pyrimidin-5-yl)-2,6-diazaspiro[3.4]octan-6-yl)pyrazin-2-yl)-1,3,4-thiadiazole (24.6 mg, 27%) as a yellow solid. ¹H NMR (400 MHz, DMSO-d₆) δ 9.68 (s, 1H), 8.57 (s, 1H), 8.18 (s, 2H), 4.16-4.05 (m, 4H), 3.93 (s, 2H), 3.72 (t, J=6.9 Hz, 2H), 2.67 (s, 3H), 2.26 (t, J=6.8 Hz, 2H). MS m/z: 435 [M+H]⁺.

### Example 1039: 6-(1-(2,2-difluoroethyl)-3-methyl-1H-pyrazolo[3,4-b]pyrazin-6-yl)-2-(2-(trifluoromethyl)pyridin-4-yl)-2,6-diazaspiro[3.4]octan-7-one

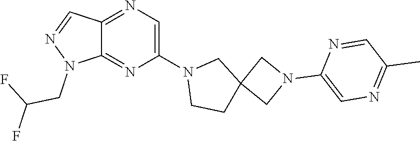 [SMILES: Cc1cnc(N2CC3(CCN(c4cnc5cnn(CC(F)F)c5n4)C3)C2)cn1]

Step 1: tert-butyl 2-(5-methylpyrazin-2-yl)-2,6-diazaspiro[3.4]octane-6-carboxylate: Followed the general procedure Y using tert-butyl 2,6-diazaspiro[3.4]octane-6-carboxylate (200 mg, 0.943 mmol, 1.0 equiv.) and 2-bromo-5-methylpyrazine (164 mg, 0.943 mmol, 1.0 equiv.) as the starting materials, XPhos Pd G3 (80.3 mg, 0.095 mmol, 0.1 equiv.) as the catalyst to give tert-butyl 2-(5-methylpyrazin-2-yl)-2,6-diazaspiro[3.4]octane-6-carboxylate (150 mg, 60%) as a white solid. MS m/z: 305 [M+H]⁺.

Step 2: 2-(5-methylpyrazin-2-yl)-2,6-diazaspiro[3.4]octane: Followed the general procedure H using tert-butyl 2-(5-methylpyrazin-2-yl)-2,6-diazaspiro[3.4]octane-6-carboxylate (150 mg) as the starting material to give the crude product 2-(5-methylpyrazin-2-yl)-2,6-diazaspiro[3.4]octane (150 mg). MS m/z: 205 [M+H]⁺.

Step 3: 1-(2,2-difluoroethyl)-6-(2-(5-methylpyrazin-2-yl)-2,6-diazaspiro[3.4]octan-6-yl)-1H-pyrazolo[3,4-b]pyrazine: Followed the general procedure I using 2-(5-methylpyrazin-2-yl)-2,6-diazaspiro[3.4]octane (150 mg, 0.489 mmol, 1.0 equiv.) and 6-chloro-1-(2,2-difluoroethyl)-1H-pyrazolo[3,4-b]pyrazine (107 mg, 0.489 mmol, 1.0 equiv.) as the starting materials to give 1-(2,2-difluoroethyl)-6-(2-(5-methylpyrazin-2-yl)-2,6-diazaspiro[3.4]octan-6-yl)-1H-pyrazolo[3,4-b]pyrazine (100.2 mg, 65%) as a white solid. ¹H NMR (300 MHz, DMSO-d₆) δ 8.12 (d, 2H), 7.97 (s, 1H), 7.82 (d, 1H), 6.41 (tt, 1H), 4.68 (td, 2H), 4.01 (q, 4H), 3.85 (s, 2H), 3.67 (t, 2H), 2.40-2.20 (m, 5H). MS m/z: 387.2 [M+H]⁺.

### Example 1040: 6-(2-(2-methyl-6-(trifluoromethyl)pyridin-4-yl)-2,6-diazaspiro[3.4]octan-6-yl)-1-(oxetan-3-yl)-1H-pyrazolo[3,4-b]pyrazine

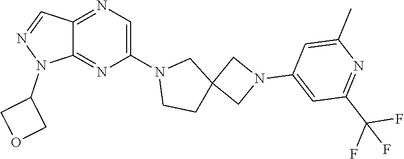 [SMILES: Cc1cc(N2CC3(CCN(c4cnc5cnn(C6COC6)c5n4)C3)C2)cc(C(F)(F)F)n1]

Step 1: tert-butyl 2-(2-methyl-6-(trifluoromethyl)pyridin-4-yl)-2,6-diazaspiro[3.4]octane-6-carboxylate: Followed the general procedure Y using tert-butyl 2,6-diazaspiro[3.4]octane-6-carboxylate (48.7 mg, 229 μmol, 1.0 equiv.) and 4-bromo-2-methyl-6-(trifluoromethyl)pyridine (50 mg, 229 μmol, 1.0 equiv.) as the starting materials to give tert-butyl 2-(2-methyl-6-(trifluoromethyl)pyridin-4-yl)-2,6-diazaspiro[3.4]octane-6-carboxylate (65 mg, 88%) as a white solid. MS m/z: 371 [M+H]⁺.

Step 2: 2-(2-methyl-6-(trifluoromethyl)pyridin-4-yl)-2,6-diazaspiro[3.4]octane hydrochloride: Followed the general procedure B using tert-butyl 2-(2-methyl-6-(trifluoromethyl)pyridin-4-yl)-2,6-diazaspiro[3.4]octane-6-carboxylate (65 mg) as the starting material to give the crude product 2-(2-methyl-6-(trifluoromethyl)pyridin-4-yl)-2,6-diazaspiro[3.4]octane hydrochloride (50 mg). MS m/z: 272 [M+H]⁺.

Step 3: 6-(2-(2-methyl-6-(trifluoromethyl)pyridin-4-yl)-2,6-diazaspiro[3.4]octan-6-yl)-1-(oxetan-3-yl)-1H-pyrazolo[3,4-b]pyrazine: Followed the general procedure I using 2-(2-methyl-6-(trifluoromethyl)pyridin-4-yl)-2,6-diazaspiro[3.4]octane hydrochloride (40 mg, 147 μmol, 1.0 equiv.) and 6-chloro-1-(oxetan-3-yl)-1H-pyrazolo[3,4-b]pyrazine (34.2 mg, 162 μmol, 1.1 equiv.) as the starting materials to give 6-(2-(2-methyl-6-(trifluoromethyl)pyridin-4-yl)-2,6-diazaspiro[3.4]octan-6-yl)-1-(oxetan-3-yl)-1H-pyrazolo[3,4-b]pyrazine (44.8 mg, 68%) as a white solid. ¹H NMR (400 MHz, DMSO-d₆) δ 8.19 (s, 1H), 8.09 (s, 1H), 6.60 (d, J=2.1 Hz, 1H), 6.45 (d, J=2.0 Hz, 1H), 5.88 (tt, J=7.9, 6.4 Hz, 1H), 5.07 (t, J=6.4 Hz, 2H), 4.97 (dd, J=7.8, 6.4 Hz, 2H), 4.12-3.95 (m, 4H), 3.85 (s, 2H), 3.66 (t, J=6.9 Hz, 2H), 2.38 (s, 3H), 2.30 (t, J=6.9 Hz, 2H). MS m/z: 446.2 [M+H]⁺.

### Example 1041: 6-(2-(3-methyl-2-(trifluoromethyl)pyridin-4-yl)-2,6-diazaspiro[3.4]octan-6-yl)-1-(oxetan-3-yl)-1H-pyrazolo[3,4-b]pyrazine

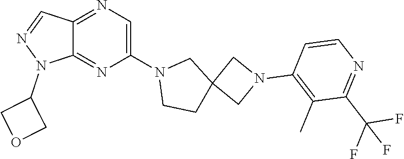 [SMILES: Cc1c(N2CC3(CCN(c4cnc5cnn(C6COC6)c5n4)C3)C2)ccnc1C(F)(F)F]

Step 1: 4-(6-(tert-butoxycarbonyl)-2,6-diazaspiro[3.4]octan-2-yl)-2-(trifluoromethyl)nicotinic acid: Followed the general procedure I using 4-chloro-2-(trifluoromethyl)nicotinic acid (350 mg, 1.55 mmol, 1.0 equiv.) and tert-butyl 2,6-diazaspiro[3.4]octane-6-carboxylate hydrochloride (386 mg, 1.55 mmol, 1.0 equiv.) as the starting materials to give 4-(6-(tert-butoxycarbonyl)-2,6-diazaspiro[3.4]octan-2-yl)-2-(trifluoromethyl)nicotinic acid (400 mg, 64%) as a white solid. MS m/z: 402 [M+H]⁺.

Step 2: tert-butyl 2-(3-(hydroxymethyl)-2-(trifluoromethyl)pyridin-4-yl)-2,6-diazaspiro[3.4]octane-6-carboxylate: Followed the general procedure Q using 4-(6-(tert-butoxycarbonyl)-2,6-diazaspiro[3.4]octan-2-yl)-2-(trifluoromethyl)nicotinic acid (400 mg, 997 μmol) as the starting material to give tert-butyl 2-(3-(hydroxymethyl)-2-(trifluoromethyl)pyridin-4-yl)-2,6-diazaspiro[3.4]octane-6-carboxylate (200 mg, 52%) as a yellow oil. MS m/z: 388 [M+H]⁺.

Step 3: tert-butyl 2-(3-methyl-2-(trifluoromethyl)pyridin-4-yl)-2,6-diazaspiro[3.4]octane-6-carboxylate. A solution of tert-butyl 2-(3-(hydroxymethyl)-2-(trifluoromethyl)pyridin-4-yl)-2,6-diazaspiro[3.4]octane-6-carboxylate (100 mg, 0.258 mmol, 1.0 equiv.) and Et₃SiH (240 mg, 2.06 mmol, 8.0 equiv.) and PdCl₂ (4.58 mg, 0.026 mmol, 0.1 equiv.) in EtOH (2 mL) was stirred for 2 h at 80° C. Desired product could be detected by LCMS. The resulting mixture was filtrated through a thin pad of Celite, and the filtrate was concentrated. The residue as purified by reverse phase Combi-flash, eluented with a gradient of MeCN: Water. This resulted in tert-butyl 2-(3-methyl-2-(trifluoromethyl)pyridin-4-yl)-2,6-diazaspiro[3.4]octane-6-carboxylate (30 mg, 31%). MS m/z: 372 [M+H]⁺.

Step 4: 2-(3-methyl-2-(trifluoromethyl)pyridin-4-yl)-2,6-diazaspiro[3.4]octane: Followed the general procedure H using tert-butyl 2-(3-methyl-2-(trifluoromethyl)pyridin-4-yl)-2,6-diazaspiro[3.4]octane-6-carboxylate (30 mg) as the starting material to give the crude product 2-(3-methyl-2-(trifluoromethyl)pyridin-4-yl)-2,6-diazaspiro[3.4]octane (30 mg). MS m/z: 272 [M+H]⁺.

Step 5: 6-(2-(3-methyl-2-(trifluoromethyl)pyridin-4-yl)-2,6-diazaspiro[3.4]octan-6-yl)-1-(oxetan-3-yl)-1H-pyrazolo[3,4-b]pyrazine: Followed the general procedure I using 2-(3-methyl-2-(trifluoromethyl)pyridin-4-yl)-2,6-diazaspiro[3.4]octane (30 mg, 0.081 mmol, 1.0 equiv.) and 6-chloro-1-(oxetan-3-yl)-1H-pyrazolo[3,4-b]pyrazine (25.6 mg, 0.121 mmol, 1.5 equiv.) as the starting materials to give 6-(2-(3-methyl-2-(trifluoromethyl)pyridin-4-yl)-2,6-diazaspiro[3.4]octan-6-yl)-1-(oxetan-3-yl)-1H-pyrazolo[3,4-b]pyrazine (16 mg, 42%) as a yellow solid. ¹H NMR (400 MHz, DMSO-d₆) δ 8.21-8.12 (m, 2H), 8.09 (s, 1H), 6.59 (d, J=5.6 Hz, 1H), 5.88 (p, J=7.2 Hz, 1H), 5.12-5.02 (m, 2H), 5.02-4.92 (m, 2H), 4.17 (q, J=8.1 Hz, 4H), 3.84 (s, 2H), 3.66 (t, J=6.9 Hz, 2H), 2.30 (t, J=7.4 Hz, 2H), 2.24 (s, 3H). MS m/z: 446.35 [M+H]⁺.

### Example 1042: 6-(2-(5-methyl-2-(trifluoromethyl)pyridin-4-yl)-2,6-diazaspiro[3.4]octan-6-yl)-1-(oxetan-3-yl)-1H-pyrazolo[3,4-b]pyrazine

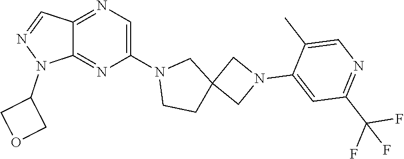 [SMILES: Cc1cnc(C(F)(F)F)cc1N1CC2(CCN(c3cnc4cnn(C5COC5)c4n3)C2)C1]

Step 1: 6-(2-(5-bromo-2-(trifluoromethyl)pyridin-4-yl)-2,6-diazaspiro[3.4]octan-6-yl)-1-(oxetan-3-yl)-1H-pyrazolo[3,4-b]pyrazine: Followed the general procedure I using 2-(5-bromo-2-(trifluoromethyl)pyridin-4-yl)-2,6-diazaspiro[3.4]octane (75 mg, 0.223 mmol, 1.0 equiv.) and 6-chloro-1-(oxetan-3-yl)-1H-pyrazolo[3,4-b]pyrazine (56.4 mg, 0.268 mmol, 1.2 equiv.) as the starting materials to give 6-(2-(5-bromo-2-(trifluoromethyl)pyridin-4-yl)-2,6-diazaspiro[3.4]octan-6-yl)-1-(oxetan-3-yl)-1H-pyrazolo[3,4-b]pyrazine (95 mg, 83%) as a white solid. MS m/z: 510 [M+H]⁺.

Step 2: 6-(2-(5-methyl-2-(trifluoromethyl)pyridin-4-yl)-2,6-diazaspiro[3.4]octan-6-yl)-1-(oxetan-3-yl)-1H-pyrazolo[3,4-b]pyrazine: Followed the general procedure L using 6-(2-(5-bromo-2-(trifluoromethyl)pyridin-4-yl)-2,6-diazaspiro[3.4]octan-6-yl)-1-(oxetan-3-yl)-1H-pyrazolo[3,4-b]pyrazine (75 mg, 0.147 mmol, 1.0 equiv.) and trimethyl-1,3,5,2,4,6-trioxatriborinane (55.3 mg, 0.441 mmol, 3.0 equiv.) as the starting materials to give 6-(2-(5-methyl-2-(trifluoromethyl)pyridin-4-yl)-2,6-diazaspiro[3.4]octan-6-yl)-1-(oxetan-3-yl)-1H-pyrazolo[3,4-b]pyrazine (16.4 mg, 23%) as a white solid. ¹H NMR (400 MHz, DMSO-d₆) δ 8.19 (s, 1H), 8.07 (d, J=13.0 Hz, 2H), 6.61 (s, 1H), 5.96-5.73 (m, 1H), 5.12-5.02 (m, 2H), 5.02-4.92 (m, 2H), 4.22 (dd, J=8.1 Hz, 4H), 3.85 (s, 2H), 3.66 (t, J=6.9 Hz, 2H), 2.30 (t, J=6.9 Hz, 2H), 2.25 (s, 3H). MS m/z: 446.15[M+H]⁺.

### Example 1043: 1-ethyl-2-phenyl-6-(2-(2-(trifluoromethyl)pyridin-4-yl)-2,6-diazaspiro[3.4]octan-6-yl)-1H-imidazo[4,5-b]pyrazine

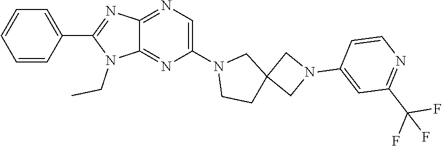 [SMILES: CCn1c(-c2ccccc2)nc2ncc(N3CCC4(CN(c5ccnc(C(F)(F)F)c5)C4)C3)nc21]

Step 1: ethyl (E)-N-(3-amino-5-bromopyrazin-2-yl)benzimidate. A solution of 5-bromopyrazine-2,3-diamine (500 mg, 2.645 mmol, 1 equiv) in (triethoxymethyl)benzene was stirred for 3 days at 100° C. under air atmosphere. The residue was purified by silica gel column chromatography, eluted with PE/EA (1:1) to ethyl (E)-N-(3-amino-5-bromopyrazin-2-yl)benzimidate (500 mg, 58%) as a white solid. MS m/z: 321 [M+H]⁺.

Step 2: 6-bromo-2-phenyl-1H-imidazo[4,5-b]pyrazine. A mixture of ethyl (E)-N-(3-amino-5-bromopyrazin-2-yl)benzimidate (500 mg, 1.557 mmol, 1 equiv) in DMF was stirred for overnight at 120° C. under air atmosphere. The resulting mixture was extracted with EtOAc (3×5 mL). The combined organic layers were washed with water (3×3 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 30 min; detector, UV 254 nm. The resulting mixture was concentrated under reduced pressure. This resulted in 6-bromo-2-phenyl-1H-imidazo[4,5-b]pyrazine (300 mg, 70%) as a white solid. MS m/z: 275 [M+H]⁺.

Step 3: 6-bromo-1-ethyl-2-phenyl-1H-imidazo[4,5-b]pyrazine: Followed the general procedure K using 6-bromo-2-phenyl-1H-imidazo[4,5-b]pyrazine (120 mg, 0.436 mmol, 1 equiv) and ethyl iodide (81.6 mg, 0.523 mmol, 1.2 equiv) as the starting materials to give 6-bromo-1-ethyl-2-phenyl-1H-imidazo[4,5-b]pyrazine (100 mg, 75%) as a white solid. MS m/z: 303 [M+H]⁺.

Step 4: 1-ethyl-2-phenyl-6-(2-(2-(trifluoromethyl)pyridin-4-yl)-2,6-diazaspiro[3.4]octan-6-yl)-1H-imidazo[4,5-b]pyrazine: Followed the general procedure Y using 6-bromo-1-ethyl-2-phenyl-1H-imidazo[4,5-b]pyrazine (98.99 mg, 0.326 mmol, 1.2 equiv) and 2-(2-(trifluoromethyl)pyridin-4-yl)-2,6-diazaspiro[3.4]octane (70 mg, 0.272 mmol, 1.00 equiv) as the starting materials, RuPhos Pd G3 (12.70 mg, 0.027 mmol, 0.1 equiv), as the catalyst to give 1-ethyl-2-phenyl-6-(2-(2-(trifluoromethyl)pyridin-4-yl)-2,6-diazaspiro[3.4]octan-6-yl)-1H-imidazo[4,5-b]pyrazine (11.8 mg, 9%) as a light yellow solid. ¹H NMR (400 MHz, Chloroform-d) δ 8.32 (d, J=5.6 Hz, 1H), 7.86 (s, 1H), 7.82-7.75 (m, 2H), 7.57-7.46 (m, 3H), 6.64 (d, J=2.3 Hz, 1H), 6.39 (dd, J=5.6, 2.3 Hz, 1H), 4.39-4.29 (m, 2H), 4.11-4.01 (m, 4H), 3.87 (s, 2H), 3.71 (t, J=6.9 Hz, 2H), 2.39 (t, J=6.9 Hz, 2H), 1.46 (t, J=7.2 Hz, 3H). MS m/z: 480.10 [M+H]⁺.

### Example 1044: 1-ethyl-2-(5-fluoropyridin-3-yl)-6-(2-(2-(trifluoromethyl)pyridin-4-yl)-2,6-diazaspiro[3.4]octan-6-yl)-1H-imidazo[4,5-b]pyrazine, Example 1045: 1-ethyl-2-(5-fluoropyridin-3-yl)-5-(2-(2-(trifluoromethyl)pyridin-4-yl)-2,6-diazaspiro[3.4]octan-6-yl)-1H-imidazo[4,5-b]pyrazine

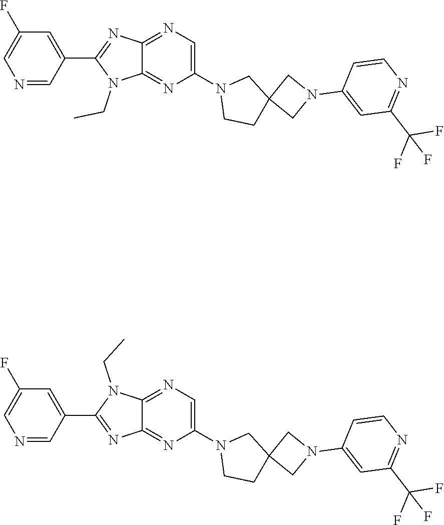 [SMILES: CCn1c(-c2cncc(F)c2)nc2ncc(N3CCC4(CN(c5ccnc(C(F)(F)F)c5)C4)C3)nc21 ; CCn1c(-c2cncc(F)c2)nc2nc(N3CCC4(CN(c5ccnc(C(F)(F)F)c5)C4)C3)cnc21]

Step 1: N-(3-amino-5-bromopyrazin-2-yl)-5-fluoronicotinamide: Followed the general procedure G using 5-fluoronicotinic acid (1 g, 7.09 mmol, 1.0 equiv.) and 5-bromopyrazine-2,3-diamine (2.13 g, 11.3 mmol, 1.6 equiv.) as the starting materials to give N-(3-amino-5-bromopyrazin-2-yl)-5-fluoronicotinamide (500 mg, 22%) as a light yellow solid. MS m/z: 312 [M+H]⁺.

Step 2: 5-bromo-2-(5-fluoropyridin-3-yl)-1H-imidazo[4,5-b]pyrazine. To a stirred mixture of N-(3-amino-5-bromopyrazin-2-yl)-5-fluoronicotinamide (500 mg, 1.60 mmol, 1.0 equiv.) in AcOH (5 mL) at 0° C. under air atmosphere. The resulting mixture was stirred for overnight at 120° C. under air atmosphere. The mixture was allowed to cool down to room temperature. The mixture was neutralized to pH 7 with saturated NaHCO₃ (aq.). The resulting mixture was extracted with DCM:MeOH=10/1 (3×60 mL). The combined organic layers were washed with brine (2×60 mL), dried over anhydrous Na₂SO₄. After filtration, the filtrate was concentrated under reduced pressure. The residue was purified by silica gel column chromatography, eluted with CH₂Cl₂/MeOH (0-30% in 20 min) to afford 5-bromo-2-(5-fluoropyridin-3-yl)-1H-imidazo[4,5-b]pyrazine (220 mg, 46%) as a yellow oil. MS m/z: 294 [M+H]⁺.

Step 3: 5-bromo-1-ethyl-2-(5-fluoropyridin-3-yl)-1H-imidazo[4,5-b]pyrazine: 6-bromo-1-ethyl-2-(5-fluoropyridin-3-yl)-1H-imidazo[4,5-b]pyrazine: Followed the general procedure K using 5-bromo-2-(5-fluoropyridin-3-yl)-1H-imidazo[4,5-b]pyrazine (220 mg, 0.75 mmol, 1.0 equiv.) and iodoethane (175 mg, 1.12 mmol, 1.5 equiv.) as the starting materials to give 5-bromo-1-ethyl-2-(5-fluoropyridin-3-yl)-1H-imidazo[4,5-b]pyrazine and 6-bromo-1-ethyl-2-(5-fluoropyridin-3-yl)-1H-imidazo[4,5-b]pyrazine two isomers (80 mg, 33%) as a yellow oil. MS m/z: 322 [M+H]⁺.

Step 4: 1-ethyl-2-(5-fluoropyridin-3-yl)-6-(2-(2-(trifluoromethyl)pyridin-4-yl)-2,6-diazaspiro[3.4]octan-6-yl)-1H-imidazo[4,5-b]pyrazine: 1-ethyl-2-(5-fluoropyridin-3-yl)-5-(2-(2-(trifluoromethyl) pyridin-4-yl)-2,6-diazaspiro[3.4]octan-6-yl)-1H-imidazo[4,5-b]pyrazine: Followed the general procedure Y using 5-bromo-1-ethyl-2-(5-fluoropyridin-3-yl)-1H-imidazo[4,5-b]pyrazine and 6-bromo-1-ethyl-2-(5-fluoropyridin-3-yl)-1H-imidazo[4,5-b]pyrazine two isomers (80 mg, 0.249 mmol, 1.0 equiv.) and 2-(2-(trifluoromethyl)pyridin-4-yl)-2,6-diazaspiro[3.4]octane hydrochloride (73 mg, 0.249 mmol, 1.0 equiv.) as the starting materials to give the crude product. The crude product was purified by Prep-HPLC to afford 1-ethyl-2-(5-fluoropyridin-3-yl)-6-(2-(2-(trifluoromethyl)pyridin-4-yl)-2,6-diazaspiro[3.4]octan-6-yl)-1H-imidazo[4,5-b]pyrazine (16.1 mg, 24%) as a yellow solid, and 1-ethyl-2-(5-fluoropyridin-3-yl)-5-(2-(2-(trifluoromethyl)pyridin-4-yl)-2,6-diazaspiro[3.4]octan-6-yl)-1H-imidazo[4,5-b]pyrazine (34.4 mg, 43%) as a yellow solid.

1-ethyl-2-(5-fluoropyridin-3-yl)-6-(2-(2-(trifluoromethyl)pyridin-4-yl)-2,6-diazaspiro[3.4]octan-6-yl)-1H-imidazo[4,5-b]pyrazine ¹H NMR (400 MHz, Chloroform-d) δ 8.84 (t, J=1.6 Hz, 1H), 8.59 (d, J=2.7 Hz, 1H), 8.32 (d, J=5.7 Hz, 1H), 7.96-7.93 (m, 1H), 7.90 (s, 1H), 6.62 (d, J=2.3 Hz, 1H), 6.40-6.38 (m, 1H), 4.37 (q, J=7.2 Hz, 2H), 4.11-4.04 (m, 4H), 3.88 (s, 2H), 3.72 (t, J=6.9 Hz, 2H), 2.41 (t, J=6.9 Hz, 2H), 1.49 (t, J=7.2 Hz, 3H). MS m/z: 499.15 [M+H]⁺.

1-ethyl-2-(5-fluoropyridin-3-yl)-5-(2-(2-(trifluoromethyl)pyridin-4-yl)-2,6-diazaspiro[3.4]octan-6-yl)-1H-imidazo[4,5-b]pyrazine ¹H NMR (400 MHz, Chloroform-d) δ 8.90 (t, J=1.5 Hz, 1H), 8.64 (d, J=2.8 Hz, 1H), 8.36 (d, J=5.7 Hz, 1H), 8.03-8.00 (m, 1H), 7.76 (s, 1H), 6.63 (d, J=2.2 Hz, 1H), 6.42-6.40 (m, 1H), 4.46 (q, J=7.2 Hz, 2H), 4.11-4.04 (m, 4H), 3.89 (s, 2H), 3.73 (t, J=6.8 Hz, 2H), 2.40 (t, J=6.9 Hz, 2H), 1.55 (t, J=7.2 Hz, 3H). MS m/z: 499.15 [M+H]⁺.

### Example 1046: 2-(6-(1-(2,2-difluoroethyl)-1H-pyrazolo[3,4-b]pyrazin-6-yl)-2,6-diazaspiro[3.4]octan-2-yl)-5-(trifluoromethyl)-1,3,4-thiadiazole

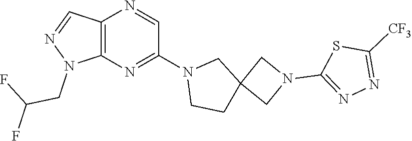 [SMILES: FC(F)Cn1ncc2ncc(N3CCC4(C3)CN(c3nnc(C(F)(F)F)s3)C4)nc21]

Step 1: 2-(6-(1-(2,2-difluoroethyl)-1H-pyrazolo[3,4-b]pyrazin-6-yl)-2,6-diazaspiro[3.4]octan-2-yl)-5-(trifluoromethyl)-1,3,4-thiadiazole: Followed the general procedure I using 2-(2,6-diazaspiro[3.4]octan-2-yl)-5-(trifluoromethyl)-1,3,4-thiadiazole (90 mg, 0.248 mmol, 1.0 equiv.) and 6-chloro-1-(2,2-difluoroethyl)-1H-pyrazolo[3,4-b]pyrazine (60 mg, 273 μmol, 1.1 equiv.) as the starting materials to give 2-(6-(1-(2,2-difluoroethyl)-1H-pyrazolo[3,4-b]pyrazin-6-yl)-2,6-diazaspiro[3.4]octan-2-yl)-5-(trifluoromethyl)-1,3,4-thiadiazole (62.7 mg, 56%) as a white solid. ¹H NMR (300 MHz, DMSO-d₆) δ 8.19-8.05 (m, 2H), 6.66-6.29 (m, 1H), 4.69 (td, J=14.9, 3.9 Hz, 2H), 4.34-4.17 (m, 4H), 3.90 (s, 2H), 3.67 (t, J=6.9 Hz, 2H), 2.36 (t, J=6.9 Hz, 2H). MS m/z: 447.15 [M+H]⁺.

### Example 1047: 2-(6-(1-(2,2-difluoroethyl)-1H-pyrazolo[3,4-b]pyrazin-6-yl)-2,6-diazaspiro[3.4]octan-2-yl)-5-methyl-4-(trifluoromethyl)thiazole

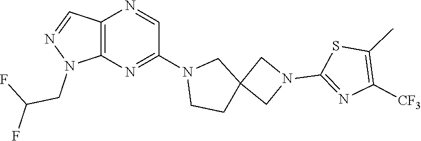 [SMILES: Cc1sc(N2CC3(CCN(c4cnc5cnn(CC(F)F)c5n4)C3)C2)nc1C(F)(F)F]

Step 1: tert-butyl 6-(1-(2,2-difluoroethyl)-1H-pyrazolo[3,4-b]pyrazin-6-yl)-2,6-diazaspiro[3.4]octane-2-carboxylate: Followed the general procedure I using 6-chloro-1-(2,2-difluoroethyl)pyrazolo[3,4-b]pyrazine (200 mg, 0.915 mmol, 1.0 equiv.) and tert-butyl 2,6-diazaspiro[3.4]octane-2-carboxylate (233 mg, 1.09 mmol, 1.2 equiv.) as the starting materials to give tert-butyl 6-(1-(2,2-difluoroethyl)-1H-pyrazolo[3,4-b]pyrazin-6-yl)-2,6-diazaspiro[3.4]octane-2-carboxylate (250 mg, 69%) as a yellow oil. MS m/z: 395 [M+H]⁺.

Step 2: 1-(2,2-difluoroethyl)-6-(2,6-diazaspiro[3.4]octan-6-yl)-1H-pyrazolo[3,4-b]pyrazine: Followed the general procedure H using 6-(1-(2,2-difluoroethyl)-1H-pyrazolo[3,4-b]pyrazin-6-yl)-2,6-diazaspiro[3.4]octane-2-carboxylate (240 mg) as the starting material to give the crude product 1-(2,2-difluoroethyl)-6-(2,6-diazaspiro[3.4]octan-6-yl)-1H-pyrazolo[3,4-b]pyrazine (200 mg). MS m/z: 295 [M+H]⁺.

Step 3: 2-(6-(1-(2,2-difluoroethyl)-1H-pyrazolo[3,4-b]pyrazin-6-yl)-2,6-diazaspiro[3.4]octan-2-yl)-5-methyl-4-(trifluoromethyl)thiazole: Followed the general procedure Y using 1-(2,2-difluoroethyl)-6-(2,6-diazaspiro[3.4]octan-6-yl)-1H-pyrazolo[3,4-b]pyrazine (200 mg, 0.680 mmol, 1.0 equiv.) and 2-bromo-5-methyl-4-(trifluoromethyl)-1,3-thiazole (167 mg, 0.680 mmol, 1.0 equiv.) as the starting materials, RuPhos Palladacycle Gen. 3 (56 mg, 0.068 mmol, 0.1 equiv.) as the catalyst to give 2-(6-(1-(2,2-difluoroethyl)-1H-pyrazolo[3,4-b]pyrazin-6-yl)-2,6-diazaspiro[3.4]octan-2-yl)-5-methyl-4-(trifluoromethyl)thiazole (39 mg, 12%) as a white solid. ¹H NMR (300 MHz, DMSO-d₆) δ 8.13 (s, 1H), 8.08 (s, 1H), 6.46 (tt, 1H), 4.76-4.61 (m, 2H), 4.08-4.00 (m, 4H), 3.85 (s, 2H), 3.65 (t, J=6.9 Hz, 2H), 2.39 (d, J=2.5 Hz, 3H), 2.30 (t, J=7.0 Hz, 2H). MS m/z: 460.15 [M+H]⁺.

### Example 1048: 1-(2,2-difluoroethyl)-6-((1R,4S)-1-ethyl-2-(2-(trifluoromethyl)pyrimidin-5-yl)-2,6-diazaspiro[3.4]octan-6-yl)-1H-pyrazolo[3,4-b]pyrazine Example 1049: 1-(2,2-difluoroethyl)-6-((1S,4S)-1-ethyl-2-(2-(trifluoromethyl)pyrimidin-5-yl)-2,6-diazaspiro[3.4]octan-6-yl)-1H-pyrazolo[3,4-b]pyrazine

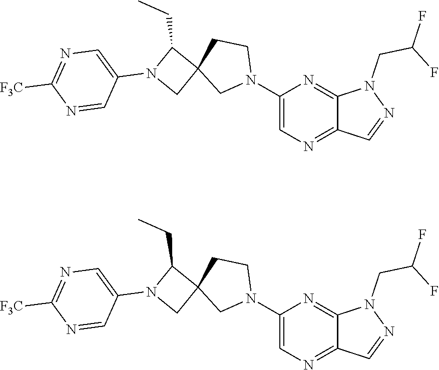 [SMILES: CC[C@H]1N(c2cnc(C(F)(F)F)nc2)C[C@]12CCN(c1cnc3cnn(CC(F)F)c3n1)C2 ; CC[C@@H]1N(c2cnc(C(F)(F)F)nc2)C[C@]12CCN(c1cnc3cnn(CC(F)F)c3n1)C2]

Step 1: (Z)-2-methyl-N-propylidenepropane-2-sulfinamide. A solution of 2-methylpropane-2-sulfinamide (6.57 g, 54.2 mmol, 1.05 equiv.), propionaldehyde (3 g, 51.6 mmol, 1.00 equiv.) and Ti(OEt)₄ (17.7 g, 77.5 mmol, 1.5 equiv) in THF (30 mL) was stirred for 4 h at 60° C. The resulting mixture was concentrated under vacuum. The residue was purified by silica gel column chromatography, eluted with PE/EA (5:1) to afford (Z)-2-methyl-N-propylidenepropane-2-sulfinamide (7 g, 84%) as a colorless oil. MS m/z: 162 [M+H]⁺.

Step 2: ethyl 1-benzyl-3-(1-((tert-butylsulfinyl)amino)propyl)pyrrolidine-3-carboxylate. A solution of ethyl 1-benzylpyrrolidine-3-carboxylate (6.94 g, 29.8 mmol, 3.0 equiv.) in THF (20 mL) was treated with LDA (39.7 mL, 39.7 mmol, 4.0 equiv.) for 1.5 h at −78° C. under nitrogen atmosphere followed by the addition of (Z)-2-methyl-N-propylidenepropane-2-sulfinamide (1.6 g, 9.9 mmol, 1.0 equiv.) dropwise at −78° C. The reaction was quenched with sat. NH₄Cl (aq.) at 0° C. The resulting mixture was extracted with EtOAc (100 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (1:1) to afford ethyl 1-benzyl-3-(1-((tert-butylsulfinyl)amino)propyl)pyrrolidine-3-carboxylate (800 mg, 20%) as a brown oil. MS m/z: 395 [M+H]⁺.

Step 3: N-(1-(1-benzyl-3-(hydroxymethyl)pyrrolidin-3-yl)propyl)-2-methylpropane-2-sulfinamide: Followed the general procedure Q using ethyl 1-benzyl-3-(1-((tert-butylsulfinyl)amino)propyl)pyrrolidine-3-carboxylate (350 mg, 0.88 mmol, 1.0 equiv.) as the starting material to give N-(1-(1-benzyl-3-(hydroxymethyl)pyrrolidin-3-yl)propyl)-2-methylpropane-2-sulfinamide (210 mg, 67%) as a colorless oil. MS m/z: 353 [M+H]⁺.

Step 4: 6-benzyl-2-(tert-butylsulfinyl)-1-ethyl-2,6-diazaspiro[3.4]octane. A solution of N-(1-(1-benzyl-3-(hydroxymethyl)pyrrolidin-3-yl)propyl)-2-methylpropane-2-sulfinamide (210 mg, 0.596 mmol, 1 equiv.), TsCl (136 mg, 0.715 mmol, 1.2 equiv.) and NaH (36 mg, 1.49 mmol, 2.5 equiv.) in DMF (3 mL) was stirred for overnight at room temperature. The resulting mixture was extracted with EtOAc (30 mL). The combined organic layers were washed with water (3×15 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 100% gradient in 30 min; detector, UV 254 nm. This resulted in 6-benzyl-2-(tert-butylsulfinyl)-1-ethyl-2,6-diazaspiro[3.4]octane (165 mg, 82.80%) as a colorless oil. MS m/z: 335 [M+H]⁺.

Step 5: 6-benzyl-1-ethyl-2,6-diazaspiro[3.4]octane hydrochloride. A solution of 6-benzyl-2-(tert-butylsulfinyl)-1-ethyl-2,6-diazaspiro[3.4]octane (165 mg, 0.493 mmol, 1.0 equiv.) and HCl(gas) in 1,4-dioxane (0.49 mL, 1.97 mmol, 4.0 equiv.) in Et₂O (2 mL) was stirred for overnight at 0° C. The resulting mixture was concentrated under vacuum. The crude product (100 mg) was used in the next step directly without further purification. MS m/z: 231 [M+H]⁺.

Step 6: 6-benzyl-1-ethyl-2-(2-(trifluoromethyl)pyrimidin-5-yl)-2,6-diazaspiro[3.4]octane: Followed the general procedure Y using 6-benzyl-1-ethyl-2,6-diazaspiro[3.4]octane hydrochloride (100 mg, 0.433 mmol, 1.0 equiv.) and 5-bromo-2-(trifluoromethyl)pyrimidine (147 mg, 0.649 mmol, 1.5 equiv.) as the starting materials to give 6-benzyl-1-ethyl-2-(2-(trifluoromethyl)pyrimidin-5-yl)-2,6-diazaspiro[3.4]octane (90 mg, 55%) as a colorless oil. MS m/z: 377 [M+H]⁺.

Step 7: 1-ethyl-2-(2-(trifluoromethyl)pyrimidin-5-yl)-2,6-diazaspiro[3.4]octane: Followed the general procedure U using 6-benzyl-1-ethyl-2-(2-(trifluoromethyl)pyrimidin-5-yl)-2,6-diazaspiro[3.4]octane (90 mg, 0.239 mmol, 1.0 equiv.) as the starting material to give 1-ethyl-2-(2-(trifluoromethyl)pyrimidin-5-yl)-2,6-diazaspiro[3.4]octane (45 mg, 66%) as a white solid. MS m/z: 287 [M+H]⁺.

Step 8: 1-(2,2-difluoroethyl)-6-(1-ethyl-2-(2-(trifluoromethyl)pyrimidin-5-yl)-2,6-diazaspiro[3.4]octan-6-yl)-1H-pyrazolo[3,4-b]pyrazine: Followed the general procedure I using 1-ethyl-2-(2-(trifluoromethyl)pyrimidin-5-yl)-2,6-diazaspiro[3.4]octane (45 mg, 0.157 mmol, 1.0 equiv.) and 5-chloro-1-(2,2-difluoroethyl)-1H-pyrazolo[3,4-b]pyrazine (38 mg, 0.173 mmol, 1.1 equiv.) as the starting materials to give 1-(2,2-difluoroethyl)-6-(1-ethyl-2-(2-(trifluoromethyl)pyrimidin-5-yl)-2,6-diazaspiro[3.4]octan-6-yl)-1H-pyrazolo[3,4-b]pyrazine (30 mg). The crude product (30 mg) was purified by Prep-HPLC with the following conditions (Column: XBridge Prep OBD C18 Column, 30*150 mm, 5 m; Mobile Phase A: 10 mmol NH₄HCO₃+0.05% NH₃H₂O, Mobile Phase B: ACN; Flow rate: 60 mL/min; Gradient: 48% B to 54% B in 10 min, 54% B; Wave Length: 254/220 nm; RT1(min): 8.57/9.35; Number Of Runs: 0) to afford 1-(2,2-difluoroethyl)-6-((1S,4S)-1-ethyl-2-(2-(trifluoromethyl)pyrimidin-5-yl)-2,6-diazaspiro[3.4]octan-6-yl)-1H-pyrazolo[3,4-b]pyrazine (9 mg, 12%) and 1-(2,2-difluoroethyl)-6-((1R,4S)-1-ethyl-2-(2-(trifluoromethyl)pyrimidin-5-yl)-2,6-diazaspiro[3.4]octan-6-yl)-1H-pyrazolo[3,4-b]pyrazine (9 mg, 12%) as a white solid.

1-(2,2-difluoroethyl)-6-((1R,4S)-1-ethyl-2-(2-(trifluoromethyl)pyrimidin-5-yl)-2,6-diazaspiro[3.4]octan-6-yl)-1H-pyrazolo[3,4-b]pyrazine MS m/z: 469.20 [M+H]⁺. ¹H NMR (400 MHz, DMSO-d₆) δ 8.25 (s, 2H), 8.11 (d, J=19.1 Hz, 2H), 6.61-6.28 (m, 1H), 4.67 (td, J=14.8, 3.9 Hz, 2H), 4.39-4.23 (m, 1H), 4.01 (dd, J=36.8, 9.7 Hz, 2H), 3.88-3.80 (m, 2H), 3.65-3.49 (m, 2H), 2.36-2.27 (m, 2H), 2.07-1.92 (m, 1H), 1.91-1.81 (m, 1H), 0.98 (t, J=7.4 Hz, 3H). MS m/z: 469.25 [M+H]⁺.

1-(2,2-difluoroethyl)-6-((1S,4S)-1-ethyl-2-(2-(trifluoromethyl)pyrimidin-5-yl)-2,6-diazaspiro[3.4]octan-6-yl)-1H-pyrazolo[3,4-b]pyrazine MS m/z: 469.20 [M+H]⁺. ¹H NMR (400 MHz, DMSO-d₆) δ 8.27-8.13 (m, 4H), 6.61-6.28 (m, 1H), 4.67 (td, J=14.8, 3.9 Hz, 2H), 4.26-4.19 (m, 1H), 4.16-4.09 (d, J=8.3 Hz, 1H), 3.96-3.80 (m, 3H), 3.66-3.56 (s, 2H), 2.37-2.17 (m, 2H), 2.04-1.92 (m, 1H), 1.89-1.77 (m, 1H), 0.88 (t, J=7.4 Hz, 3H). MS m/z: 469.2 [M+H]⁺.

### Example 1050: 1-(2,2-difluoroethyl)-5-ethyl-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one

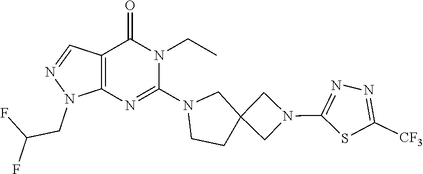 [SMILES: CCn1c(N2CCC3(CN(c4nnc(C(F)(F)F)s4)C3)C2)nc2c(cnn2CC(F)F)c1=O]

Step 1: tert-butyl 2-(5-(trifluoromethyl)-1,3,4-thiadiazol-2-yl)-2,6-diazaspiro[3.4]octane-6-carboxylate: Followed the general procedure I using tert-butyl 2,6-diazaspiro[3.4]octane-6-carboxylate (500 mg, 2.35 mmol, 1.0 equiv.) and 2-chloro-5-(trifluoromethyl)-1,3,4-thiadiazole (488 mg, 2.59 mmol, 1.1 equiv) as the starting materials to give tert-butyl 2-(2-methyl-6-(trifluoromethyl)pyrimidin-4-yl)-2,8-diazaspiro[4.5]decane-8-carboxylate (380 mg, 44%) as a yellow oil. MS m/z: 365 [M+H]⁺.

Step 2: 2-(2,6-diazaspiro[3.4]octan-2-yl)-5-(trifluoromethyl)-1,3,4-thiadiazole: Followed the general procedure H using tert-butyl 2-(2-methyl-6-(trifluoromethyl)pyrimidin-4-yl)-2,8-diazaspiro[4.5]decane-8-carboxylate (370 mg) as the starting material to give the crude product 2-(2,6-diazaspiro[3.4]octan-2-yl)-5-(trifluoromethyl)-1,3,4-thiadiazole (300 mg). MS m/z: 265 [H+M]⁺.

Step 3: 5-ethyl-1-(tetrahydro-2H-pyran-2-yl)-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one: Followed the general procedure I using 2-(2,6-diazaspiro[3.4]octan-2-yl)-5-(trifluoromethyl)-1,3,4-thiadiazole (100 mg, 0.276 mmol, 1.0 equiv.) and 6-chloro-5-ethyl-1-(oxan-2-yl)pyrazolo[3,4-d]pyrimidin-4-one (93 mg, 0.331 mmol, 1.2 equiv.) as the starting materials to give 5-ethyl-1-(tetrahydro-2H-pyran-2-yl)-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one (100 mg, 70%) as a yellow oil. MS m/z: 511 [M+H]⁺.

Step 4: 5-ethyl-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one: Followed the general procedure H using 5-ethyl-1-(tetrahydro-2H-pyran-2-yl)-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one (100 mg) as the starting material to give the crude product 5-ethyl-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one (70 mg, 83%). MS m/z: 427 [M+H]⁺.

Step 5: 1-(2,2-difluoroethyl)-5-ethyl-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one: Followed the general procedure K using 5-ethyl-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one (70 mg, 0.164 mmol, 1.0 equiv.) and 2,2-difluoroethyl trifluoromethanesulfonate (38 mg, 180 μmol, 1.1 equiv.) as the starting materials to give 1-(2,2-difluoroethyl)-5-ethyl-6-(2-(5-(trifluoromethyl)-1,3,4-thiadiazol-2-yl)-2,6-diazaspiro[3.4]octan-6-yl)-1,5-dihydro-4H-pyrazolo[3,4-d]pyrimidin-4-one (20.6 mg, 25%) as a white solid. ¹H NMR (300 MHz, DMSO-d₆) δ 7.95 (s, 1H), 6.43 (tt, J=55.0, 4.0 Hz, 1H), 4.58 (td, J=14.9, 3.9 Hz, 2H), 4.28 (d, J=8.4 Hz, 2H), 4.19 (d, J=8.4 Hz, 2H), 4.04 (q, J=6.9 Hz, 2H), 3.85 (s, 2H), 3.66 (d, J [002468]=6.8 Hz, 2H), 2.26 (t, J=6.7 Hz, 2H), 1.27 (t, J=6.9 Hz, 3H). MS m/z: 491.25 [M+H]⁺.

### Example 1051: 2-(6-(1-(2,2-difluoroethyl)-1H-pyrazolo[3,4-d]pyrimidin-6-yl)-2,6-diazaspiro[3.4]octan-2-yl)-5-(trifluoromethyl)-1,3,4-thiadiazole

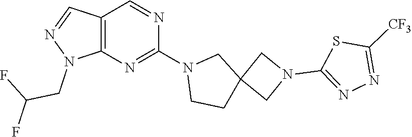 [SMILES: FC(F)Cn1ncc2cnc(N3CCC4(C3)CN(c3nnc(C(F)(F)F)s3)C4)nc21]

Step 1: 2-(6-(1-(2,2-difluoroethyl)-1H-pyrazolo[3,4-d]pyrimidin-6-yl)-2,6-diazasp: Followed the general procedure I using 6-chloro-1-(2,2-difluoroethyl)-1H-pyrazolo[3,4-d]pyrimidine (90 mg, 0.412 mmol, 1.0 equiv.) and 2-(2,6-diazaspiro[3.4]octan-2-yl)-5-(trifluoromethyl)-1,3,4-thiadiazole (180 mg, 453 μmol, 1.1 equiv.) as the starting materials to give 2-(6-(1-(2,2-difluoroethyl)-1H-pyrazolo[3,4-d]pyrimidin-6-yl)-2,6-diazasp (30 mg, 16%) as a white solid. ¹H NMR (300 MHz, DMSO-d₆) δ 8.93 (s, 1H), 8.09 (s, 1H), 6.66-6.28 (m, 1H), 4.72-4.60 (m, 2H), 4.30-4.17 (m, 4H), 3.88 (s, 2H), 3.66 (br, 2H), 2.32 (t, J=6.9 Hz, 2H). MS m/z: 447.20 [M+H]⁺.

### Example 1052: 2-methyl-5-(6-(2-(2-(trifluoromethyl)pyridin-4-yl)-2,6-diazaspiro[3.4]octan-6-yl)pyrazin-2-yl)-1,3,4-thiadiazole

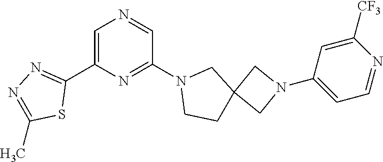 [SMILES: Cc1nnc(-c2cncc(N3CCC4(CN(c5ccnc(C(F)(F)F)c5)C4)C3)n2)s1]

Step 1: tert-butyl 6-(6-(methoxycarbonyl)pyrazin-2-yl)-2,6-diazaspiro[3.4]octane-2-carboxylate: Followed the general procedure I using (500 mg, 2.89 mmol, 1.0 equiv.) and tert-butyl 2,6-diazaspiro[3.4]octane-2-carboxylate (739 mg, 3.47 mmol, 1.2 equiv.) as the starting materials to give tert-butyl 6-(6-(methoxycarbonyl)pyrazin-2-yl)-2,6-diazaspiro[3.4]octane-2-carboxylate (650 mg, 64%) as a white solid. MS m/z: 349 [M+H]⁺.

Step 2: tert-butyl 6-(6-(hydrazinecarbonyl)pyrazin-2-yl)-2,6-diazaspiro[3.4]octane-2-carboxylate: Followed the general procedure S using tert-butyl 6-(6-(methoxycarbonyl)pyrazin-2-yl)-2,6-diazaspiro[3.4]octane-2-carboxylate (650 mg, 1.87 mmol, 1.0 equiv.) as the starting material to give tert-butyl 6-(6-(hydrazinecarbonyl)pyrazin-2-yl)-2,6-diazaspiro[3.4]octane-2-carboxylate (500 mg, 77.0%) as a white solid. MS m/z: 349 [M+H]⁺

Step 3: tert-butyl 6-(6-(2-acetylhydrazine-1-carbonyl)pyrazin-2-yl)-2,6-diazaspiro[3.4]octane-2-carboxylate: Followed the general procedure AF using tert-butyl 6-(6-(hydrazinecarbonyl)pyrazin-2-yl)-2,6-diazaspiro[3.4]octane-2-carboxylate (480 mg, 1.38 mmol, 1.0 equiv.) as the starting material to give tert-butyl 6-(6-(2-acetylhydrazine-1-carbonyl)pyrazin-2-yl)-2,6-diazaspiro[3.4]octane-2-carboxylate (400 mg, 74%) as a yellow solid. MS m/z: 391 [M+H]⁺

Step 4: tert-butyl 6-(6-(5-methyl-1,3,4-thiadiazol-2-yl)pyrazin-2-yl)-2,6-diazaspiro[3.4]octane-2-carboxylate: Followed the general procedure T using tert-butyl 6-(6-(2-formylhydrazine-1-carbonyl)-5-methoxypyrazin-2-yl)-2,6-diazaspiro[3.4]octane-2-carboxylate (400 mg, 1.03 mmol, 1.0 equiv.) as the starting material to give tert-butyl 6-(6-(5-methyl-1,3,4-thiadiazol-2-yl)pyrazin-2-yl)-2,6-diazaspiro[3.4]octane-2-carboxylate (100 mg, 25%) as a yellow green solid. MS m/z: 389 [M+H]⁺.

Step 5: 2-(6-(2,6-diazaspiro[3.4]octan-6-yl)pyrazin-2-yl)-5-methyl-1,3,4-thiadiazole: Followed the general procedure H using tert-butyl 6-(6-(5-methyl-1,3,4-thiadiazol-2-yl)pyrazin-2-yl)-2,6-diazaspiro[3.4]octane-2-carboxylate (100 mg) as the starting material to give the crude product 2-(6-(2,6-diazaspiro[3.4]octan-6-yl)pyrazin-2-yl)-5-methyl-1,3,4-thiadiazole (60 mg). MS m/z: 289 [M+H]⁺.

Step 6: 2-methyl-5-(6-(2-(2-(trifluoromethyl)pyridin-4-yl)-2,6-diazaspiro[3.4]octan-6-yl)pyrazin-2-yl)-1,3,4-thiadiazole: Followed the general procedure I using 2-(6-(2,6-diazaspiro[3.4]octan-6-yl)pyrazin-2-yl)-5-methyl-1,3,4-thiadiazole (60 mg, 0.208 mmol, 1.0 equiv.) and 4-chloro-2-(trifluoromethyl)pyridine (45.6 mg, 0.249 mmol, 1.2 equiv.) as the starting materials to give 2-methyl-5-(6-(2-(2-(trifluoromethyl)pyridin-4-yl)-2,6-diazaspiro[3.4]octan-6-yl)pyrazin-2-yl)-1,3,4-thiadiazole (18.1 mg, 20%) as a yellow solid. ¹H NMR (400 MHz, DMSO-d₆) δ 8.56 (s, 1H), 8.25 (d, J=5.6 Hz, 1H), 8.13 (s, 1H), 6.76 (d, J=2.2 Hz, 1H), 6.58 (dd, J=5.7, 2.3 Hz, 1H), 4.09-3.99 (m, 4H), 3.79 (s, 2H), 3.60 (t, J=6.9 Hz, 2H), 2.78 (s, 3H), 2.31 (t, J=6.9 Hz, 2H). MS m/z: 434.1 [M+H]⁺.

### Example 1053: 1-(2,2-difluoroethyl)-5-methyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one

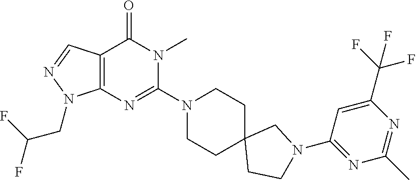 [SMILES: Cc1nc(N2CCC3(CCN(c4nc5c(cnn5CC(F)F)c(=O)n4C)CC3)C2)cc(C(F)(F)F)n1]

Step 1: tert-butyl 2-(2-methyl-6-(trifluoromethyl)pyrimidin-4-yl)-2,8-diazaspiro[4.5]decane-8-carboxylate: Followed the general procedure I using 4-chloro-2-methyl-6-(trifluoromethyl)pyrimidine (100 mg, 0.51 mmol, 1.0 equiv.) and tert-butyl 2,8-diazaspiro[4.5]decane-8-carboxylate (534 mg, 0.56 mmol, 1.1 equiv.) as the starting materials to give tert-butyl 2-(2-methyl-6-(trifluoromethyl)pyrimidin-4-yl)-2,8-diazaspiro[4.5]decane-8-carboxylate (90 mg, 32%) as a yellow solid. MS m/z: 401 [M+H]⁺.

Step 2: 2-(2-methyl-6-(trifluoromethyl)pyrimidin-4-yl)-2,8-diazaspiro[4.5]decane hydrochloride: Followed the general procedure B using tert-butyl 2-(2-methyl-6-(trifluoromethyl)pyrimidin-4-yl)-2,8-diazaspiro[4.5]decane-8-carboxylate (90 mg) as the starting material to give the crude product 2-(2-methyl-6-(trifluoromethyl)pyrimidin-4-yl)-2,8-diazaspiro[4.5]decane hydrochloride (90 mg). MS m/z: 301 [M+H]⁺.

Step 3: 1-(2,2-difluoroethyl)-5-methyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure I using 2-(2-methyl-6-(trifluoromethyl)pyrimidin-4-yl)-2,8-diazaspiro[4.5]decane hydrochloride (90 mg, 0.30 mmol, 1.0 equiv.) and 6-chloro-1-(2,2-difluoroethyl)-5-methyl-1,5-dihydro-4H-pyrazolo[3,4-d]pyrimidin-4-one (81.8 mg, 0.33 mmol, 1.1 equiv.) as the starting materials to give 1-(2,2-difluoroethyl)-5-methyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (46.4 mg, 92%) as a white solid. ¹H NMR (400 MHz, DMSO-d₆) δ 8.02 (s, 1H), 6.76 (d, J=42.2 Hz, 1H), 6.44 (tt, J=54.8, 3.9 Hz, 1H), 4.63 (t, J=14.7 Hz, 2H), 3.65 (t, J=7.0 Hz, 1H), 3.53 (d, J=11.6 Hz, 2H), 3.44 (s, 4H), 3.38 (s, 1H), 3.20 (s, 3H), 2.43 (s, 3H), 1.98 (t, J=7.1 Hz, 1H), 1.90 (t, J=7.1 Hz, 1H), 1.73 (s, 4H). MS m/z: 513.15[M+H]⁺.

### Example 1054: 1-(2,2-difluoroethyl)-5-methyl-6-(2-(6-(trifluoromethyl)pyridin-3-yl)-2,8-diazaspiro[4.5]decan-8-yl)-1,5-dihydro-4H-pyrazolo[3,4-d]pyrimidin-4-one

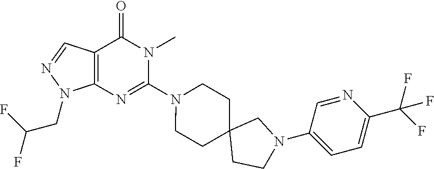 [SMILES: Cn1c(N2CCC3(CCN(c4ccc(C(F)(F)F)nc4)C3)CC2)nc2c(cnn2CC(F)F)c1=O]

Step 1: tert-butyl 2-(6-(trifluoromethyl)pyridin-3-yl)-2,8-diazaspiro[4.5]decane-8-carboxylate: Followed the general procedure I using tert-butyl 2,8-diazaspiro[4.5]decane-8-carboxylate (100 mg, 0.415 mmol, 1.0 equiv.) and 5-fluoro-2-(trifluoromethyl)pyridine (100 mg, 0.415 mmol, 1.0 equiv.) as the starting materials to give tert-butyl 2-(6-(trifluoromethyl)pyridin-3-yl)-2,8-diazaspiro[4.5]decane-8-carboxylate (140 mg, 70%) as a white solid. MS m/z: 386 [M+H]⁺.

Step 2: 2-(6-(trifluoromethyl)pyridin-3-yl)-2,8-diazaspiro[4.5]decane hydrochloride: Followed the general procedure B using tert-butyl 2-(6-(trifluoromethyl)pyridin-3-yl)-2,8-diazaspiro[4.5]decane-8-carboxylate (140 mg) as the starting material to give the crude product 2-(6-(trifluoromethyl)pyridin-3-yl)-2,8-diazaspiro[4.5]decane hydrochloride (140 mg). MS m/z: 286 [M+H]⁺.

Step 3: 5-methyl-1-(tetrahydro-2H-pyran-2-yl)-6-(2-(6-(trifluoromethyl)pyridin-3-yl)-2,8-diazaspiro[4.5]decan-8-yl)-1,5-dihydro-4H-pyrazolo[3,4-d]pyrimidin-4-one: Followed the general procedure I using 2-(6-(trifluoromethyl)pyridin-3-yl)-2,8-diazaspiro[4.5]decane hydrochloride (140 mg, 0.490 mmol, 1.0 equiv.) and 6-chloro-5-methyl-1-(tetrahydro-2H-pyran-2-yl)-1,5-dihydro-4H-pyrazolo[3,4-d]pyrimidin-4-one (70 mg, 0.261 mmol, 1.0 equiv.) as the starting materials to give 5-methyl-1-(tetrahydro-2H-pyran-2-yl)-6-(2-(6-(trifluoromethyl)pyridin-3-yl)-2,8-diazaspiro[4.5]decan-8-yl)-1,5-dihydro-4H-pyrazolo[3,4-d]pyrimidin-4-one (110 mg, 60%) as a white solid. MS m/z: 518 [M+H]⁺.

Step 4: 5-methyl-6-(2-(6-(trifluoromethyl)pyridin-3-yl)-2,8-diazaspiro[4.5]decan-8-yl)-1,5-dihydro-4H-pyrazolo[3,4-d]pyrimidin-4-one: Followed the general procedure M using 5-methyl-1-(tetrahydro-2H-pyran-2-yl)-6-(2-(6-(trifluoromethyl)pyridin-3-yl)-2,8-diazaspiro[4.5]decan-8-yl)-1,5-dihydro-4H-pyrazolo[3,4-d]pyrimidin-4-one (110 mg, 0.212 mmol, 1.0 equiv.) as the starting material to give 5-methyl-6-(2-(6-(trifluoromethyl)pyridin-3-yl)-2,8-diazaspiro[4.5]decan-8-yl)-1,5-dihydro-4H-pyrazolo[3,4-d]pyrimidin-4-one (70 mg, 65%) as a white solid. MS m/z: 434 [M+H]⁺.

Step 5: 1-(2,2-difluoroethyl)-5-methyl-6-(2-(6-(trifluoromethyl)pyridin-3-yl)-2,8-diazaspiro[4.5]decan-8-yl)-1,5-dihydro-4H-pyrazolo[3,4-d]pyrimidin-4-one: Followed the general procedure K using 5-methyl-6-(2-(6-(trifluoromethyl)pyridin-3-yl)-2,8-diazaspiro[4.5]decan-8-yl)-1,5-dihydro-4H-pyrazolo[3,4-d]pyrimidin-4-one (70.0 mg, 0.162 mmol, 1.0 equiv.) and CHF₂CH₂OTf (69.4 mg, 0.324 mmol, 2.0 equiv.) as the starting materials to give 1-(2,2-difluoroethyl)-5-methyl-6-(2-(6-(trifluoromethyl)pyridin-3-yl)-2,8-diazaspiro[4.5]decan-8-yl)-1,5-dihydro-4H-pyrazolo[3,4-d]pyrimidin-4-one (20.2 mg, 35%) as a white solid. ¹H NMR (300 MHz, Chloroform-d) δ 8.03 (d, 2H), 7.51 (d, 1H), 6.91-6.82 (m, 1H), 6.20 (tt, 1H), 4.59 (td, 2H), 3.61-3.41 (m, 5H), 3.39-3.15 (m, 6H), 2.06 (t, 2H), 1.99-1.85 (m, 4H). MS m/z: 498 [M+H]⁺.

### Example 1055: 8-(1-(oxetan-3-yl)-1H-pyrazolo[3,4-b]pyrazin-6-yl)-2-(6-(trifluoromethyl)pyridazin-4-yl)-2,8-diazaspiro[4.5]decane

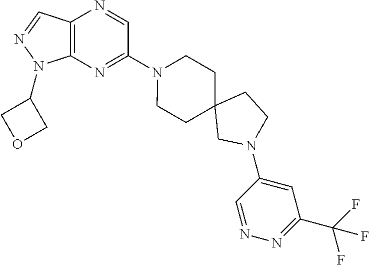 [SMILES: FC(F)(F)c1cc(N2CCC3(CCN(c4cnc5cnn(C6COC6)c5n4)CC3)C2)cnn1]

Step 1: 4-bromo-6-(trifluoromethyl)pyridazin-3-amine. To a stirred solution of 6-(trifluoromethyl)pyridazin-3-amine (1000 mg, 6.13 mmol, 1.0 equiv.) and NaHCO₃ (1.55 g, 18.3 mmol, 3.0 equiv.) in MeOH (10 mL) was added Br₂ (980 mg, 6.13 mmol, 1.0 equiv) dropwise at 0° C. The resulting mixture was stirred for 2 h at room temperature. Desired product could be detected by LCMS. The resulting mixture was diluted with EtOAc (30 mL), washed with water and brine, dried over anhydrous Na₂SO₄. After filtration, the filtrate was concentrated under reduced pressure. The residue was purified with Combi-flash (40 g silico gel column), eluted with Hex:EtOAc to afford 4-bromo-6-(trifluoromethyl)pyridazin-3-amine (750 mg, 42%) as a yellow solid. MS m/z: 242 [M+H]⁺.

Step 2: tert-butyl 2-[3-amino-6-(trifluoromethyl)pyridazin-4-yl]-2,8-diazaspiro[4.5]decane-8-carboxylate: Followed the general procedure I using 4-bromo-6-(trifluoromethyl)pyridazin-3-amine (750 mg, 3.10 mmol, 1.0 equiv.) and tert-butyl 2,8-diazaspiro[4.5]decane-8-carboxylate (744 mg, 3.10 mmol, 1.0 equiv.) as the starting materials to give tert-butyl 2-[3-amino-6-(trifluoromethyl)pyridazin-4-yl]-2,8-diazaspiro[4.5]decane-8-carboxylate (700 mg, 56%) as a white solid. MS m/z: 402 [M+H]⁺.

Step 3: tert-butyl 2-[6-(trifluoromethyl)pyridazin-4-yl]-2,8-diazaspiro[4.5]decane-8-carboxylate. To a stirred solution of tert-butyl 2-[3-amino-6-(trifluoromethyl)pyridazin-4-yl]-2,8-diazaspiro[4.5]decane-8-carboxylate (700 mg, 1.75 mmol, 1.0 equiv) and tert-butyl nitrite (360 mg, 3.50 mmol, 2.0 equiv) in THF (10 mL) was added salicyclic acid (23.4 mg, 0.17 mmol, 0.10 equiv) at 0° C. The resulting mixture was stirred overnight at room temperature. Desired product could be detected by LCMS. The resulting mixture was concentrated under vacuum. The residue was purified with Combi-flash (40 g silico gel column), eluted with gradient of Hex:EtOAc to afford tert-butyl 2-[6-(trifluoromethyl)pyridazin-4-yl]-2,8-diazaspiro[4.5]decane-8-carboxylate (120 mg, 17%) as a colorless semi-solid. MS m/z: 387 [M+H]⁺.

Step 4: 2-[6-(trifluoromethyl)pyridazin-4-yl]-2,8-diazaspiro[4.5]decane hydrochloride: Followed the general procedure B using tert-butyl 2-[6-(trifluoromethyl)pyridazin-4-yl]-2,8-diazaspiro[4.5]decane-8-carboxylate (120 mg) as the starting material to give the crude product 2-[6-(trifluoromethyl)pyridazin-4-yl]-2,8-diazaspiro[4.5]decane hydrochloride (120 mg). MS m/z: 287 [M+H]⁺.

Step 5: 8-(1-(oxetan-3-yl)-1H-pyrazolo[3,4-b]pyrazin-6-yl)-2-(6-(trifluoromethyl)pyridazin-4-yl)-2,8-diazaspiro[4.5]decane: Followed the general procedure I using 2-[6-(trifluoromethyl)pyridazin-4-yl]-2,8-diazaspiro[4.5]decane hydrochloride (60 mg, 0.155 mmol, 1.0 equiv.) and 6-chloro-1-(oxetan-3-yl)-1H-pyrazolo[3,4-b]pyrazine (32.6 mg, 0.155 mmol, 1.0 equiv.) as the starting materials to give 8-(1-(oxetan-3-yl)-1H-pyrazolo[3,4-b]pyrazin-6-yl)-2-(6-(trifluoromethyl)pyridazin-4-yl)-2,8-diazaspiro[4.5]decane (11.5 mg, 16.0%) as a white solid. ¹H NMR (400 MHz, Chloroform-d) δ 8.77 (s, 1H), 8.26 (s, 1H), 8.11 (s, 1H), 6.76 (s, 1H), 5.93 (p, J=7.3 Hz, 1H), 5.34 (t, J=6.6 Hz, 2H), 5.06 (dd, J=7.9, 6.6 Hz, 2H), 3.83 (dtd, J=19.4, 13.5, 5.0 Hz, 4H), 3.63 (br, 1H), 3.42 (br, 1H), 2.12 (t, J=6.9 Hz, 2H), 1.82 (t, J=5.3 Hz, 4H). MS m/z: 461.15 [M+H]⁺.

### Example 1056: 2-(4-methyl-2-(trifluoromethyl)pyrimidin-5-yl)-8-(1-(oxetan-3-yl)-1H-pyrazolo[3,4-b]pyrazin-6-yl)-2,8-diazaspiro[4.5]decane

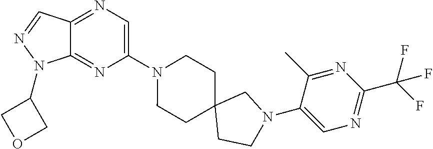 [SMILES: Cc1nc(C(F)(F)F)ncc1N1CCC2(CCN(c3cnc4cnn(C5COC5)c4n3)CC2)C1]

Step 1: 2-(4-methyl-2-(trifluoromethyl)pyrimidin-5-yl)-8-(1-(oxetan-3-yl)-1H-pyrazolo[3,4-b]pyrazin-6-yl)-2,8-diazaspiro[4.5]decane: Followed the general procedure I using 2-(4-methyl-2-(trifluoromethyl)pyrimidin-5-yl)-2,8-diazaspiro[4.5]decane hydrochloride (30 mg, 0.10 mmol, 1.00 equiv.) and 6-chloro-1-(oxetan-3-yl)-1H-pyrazolo[3,4-b]pyrazine (21.1 mg, 0.10 mmol, 1.00 equiv.) as the starting materials to give 2-(4-methyl-2-(trifluoromethyl)pyrimidin-5-yl)-8-(1-(oxetan-3-yl)-1H-pyrazolo[3,4-b]pyrazin-6-yl)-2,8-diazaspiro[4.5]decane (36.0 mg, 76%) as a white solid. ¹H NMR (400 MHz, DMSO-d₆) δ 8.48 (s, 1H), 8.16 (d, 2H), 5.89 (h, 1H), 5.06 (t, 2H), 5.00-4.91 (m, 2H), 3.94-3.70 (m, 4H), 3.60 (t, 2H), 3.42 (s, 2H), 2.65 (s, 3H), 1.91 (t, 2H), 1.76-1.59 (m, 4H). MS m/z: 475.15 [M+H]⁺.

### Example 1057: 5-methyl-1-(oxetan-3-yl)-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one

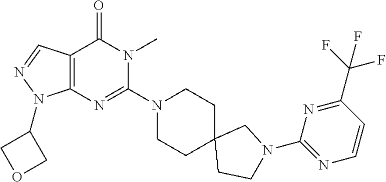 [SMILES: Cn1c(N2CCC3(CCN(c4nccc(C(F)(F)F)n4)C3)CC2)nc2c(cnn2C2COC2)c1=O]

Step 1: tert-butyl 2-(4-(trifluoromethyl)pyrimidin-2-yl)-2,8-diazaspiro[4.5]decane-8-carboxylate: Followed the general procedure I using tert-butyl 2,8-diazaspiro[4.5]decane-8-carboxylate (100 mg, 0.415 mmol, 1.0 equiv.) and 2-chloro-4-(trifluoromethyl)pyrimidine (76 mg, 0.415 mmol, 1.0 equiv.) as the starting materials to give tert-butyl 2-(4-(trifluoromethyl)pyrimidin-2-yl)-2,8-diazaspiro[4.5]decane-8-carboxylate (120 mg, 75%) as a white solid. MS m/z: 387 [M+H]⁺.

Step 2: 2-(4-(trifluoromethyl)pyrimidin-2-yl)-2,8-diazaspiro[4.5]decane hydrochloride: Followed the general procedure B using tert-butyl 2-(4-(trifluoromethyl)pyrimidin-2-yl)-2,8-diazaspiro[4.5]decane-8-carboxylate (120 mg) as the starting material to give the crude product 2-(4-(trifluoromethyl)pyrimidin-2-yl)-2,8-diazaspiro[4.5]decane hydrochloride (120 mg). MS m/z: 287 [M+H]⁺.

Step 3: 5-methyl-1-(tetrahydro-2H-pyran-2-yl)-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one: Followed the general procedure I using 2-(4-(trifluoromethyl)pyrimidin-2-yl)-2,8-diazaspiro[4.5]decane hydrochloride (120 mg, 0.418 mmol, 1.0 equiv.) and 6-chloro-5-methyl-1-(tetrahydro-2H-pyran-2-yl)-1,5-dihydro-4H-pyrazolo[3,4-d]pyrimidin-4-one (112 mg, 0.418 mmol, 1.0 equiv.) as the starting materials to give 5-methyl-1-(tetrahydro-2H-pyran-2-yl)-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one (100 mg, 50%) as a white solid. MS m/z: 519 [M+H]⁺.

Step 4: 5-methyl-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one: Followed the general procedure M using 5-methyl-1-(tetrahydro-2H-pyran-2-yl)-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one (100 mg, 0.193 mmol, 1.0 equiv.) as the starting material to give 5-methyl-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one (80 mg, 70%) as a white solid. MS m/z: 435 [M+H]⁺.

Step 5: 5-methyl-1-(oxetan-3-yl)-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one: Followed the general procedure K using 5-methyl-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one (80 mg, 0.184 mmol, 1.0 equiv.) and oxetan-3-yl trifluoromethanesulfonate (114 mg, 0.552 mmol, 1.0 equiv.) as the starting materials to give 5-methyl-1-(oxetan-3-yl)-6-(2-(4-(trifluoromethyl)pyrimidin-2-yl)-2,8-diazaspiro[4.5]decan-8-yl)-1,5-dihydro-4H-pyrazolo[3,4-d]pyrimidin-4-one (17.2 mg, 20%) as a white solid. ¹H NMR (300 MHz, Chloroform-d) δ 8.53 (d, 1H), 8.09 (s, 1H), 6.81 (d, 1H), 5.85 (q, 1H), 5.30 (t, 2H), 5.11-5.01 (m, 2H), 3.75 (t, 2H), 3.61 (s, 2H), 3.55 (s, 3H), 3.40-3.22 (m, 4H), 2.01 (t, 2H), 1.86-1.79 (m, 4H). MS m/z: 491.2 [M+H]⁺.

### Example 1058: 5-methyl-1-(oxetan-3-yl)-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one

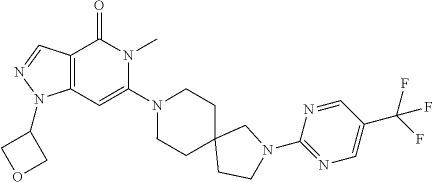 [SMILES: Cn1c(N2CCC3(CCN(c4ncc(C(F)(F)F)cn4)C3)CC2)cc2c(cnn2C2COC2)c1=O]

Step 1: tert-butyl 2-(5-(trifluoromethyl)pyrimidin-2-yl)-2,8-diazaspiro[4.5]decane-8-carboxylate: Followed the general procedure I using tert-butyl 2,8-diazaspiro[4.5]decane-8-carboxylate (100 mg, 0.415 mmol, 1.0 equiv.) and 2-chloro-5-(trifluoromethyl)pyrimidine (75.9 mg, 0.415 mmol, 1.0 equiv.) as the starting materials to give tert-butyl 2-(5-(trifluoromethyl)pyrimidin-2-yl)-2,8-diazaspiro[4.5]decane-8-carboxylate (130 mg, 60%) as a white solid. MS m/z: 387 [M+H]⁺.

Step 2: 2-(5-(trifluoromethyl)pyrimidin-2-yl)-2,8-diazaspiro[4.5]decane hydrochloride: Followed the general procedure B using tert-butyl 2-(5-(trifluoromethyl)pyrimidin-2-yl)-2,8-diazaspiro[4.5]decane-8-carboxylate (130 mg) as the starting material to give the crude product 2-(5-(trifluoromethyl)pyrimidin-2-yl)-2,8-diazaspiro[4.5]decane hydrochloride (130 mg). MS m/z: 287 [M+H]⁺.

Step 3: 5-methyl-1-(tetrahydro-2H-pyran-2-yl)-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one: Followed the general procedure I using 2-(5-(trifluoromethyl)pyrimidin-2-yl)-2,8-diazaspiro[4.5]decane hydrochloride (130 mg, 0.453 mmol, 1.8 equiv.) and 6-chloro-5-methyl-1-(tetrahydro-2H-pyran-2-yl)-1,5-dihydro-4H-pyrazolo[3,4-d]pyrimidin-4-one (65.6 mg, 0.244 mmol, 1.0 equiv.) as the starting materials to give 5-methyl-1-(tetrahydro-2H-pyran-2-yl)-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one (100 mg, 70%) as a white solid. MS m/z: 519 [M+H]⁺.

Step 4: 5-methyl-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one: Followed the general procedure M using 5-methyl-1-(tetrahydro-2H-pyran-2-yl)-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one (100 mg, 0.193 mmol, 1.0 equiv.) as the starting material to give 5-methyl-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one (70.0 mg, 85%) as a white solid. MS m/z: 435 [M+H]⁺.

Step 5: 5-methyl-1-(oxetan-3-yl)-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one: Followed the general procedure K using 5-methyl-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one (70.0 mg, 0.161 mmol, 1.0 equiv.) and oxetan-3-yl trifluoromethanesulfonate (100 mg, 0.483 mmol, 1.0 equiv.) as the starting materials to give 5-methyl-1-(oxetan-3-yl)-6-(2-(5-(trifluoromethyl)pyrimidin-2-yl)-2,8-diazaspiro[4.5]decan-8-yl)-1,5-dihydro-4H-pyrazolo[3,4-d]pyrimidin-4-one (13.5 mg, 30%) as a white solid. ¹H NMR (400 MHz, Chloroform-d) δ 8.52 (d, 2H), 8.07 (s, 1H), 5.89-5.78 (m, 1H), 5.28 (t, 2H), 5.03 (dd, 2H), 3.75 (t, 2H), 3.61 (s, 2H), 3.53 (s, 3H), 3.37-3.25 (m, 2H), 3.26-3.14 (m, 2H), 2.01 (t, 2H), 1.82 (t, 4H). MS m/z: 491.05 [M+H]⁺.

### Example 1059: 5-methyl-1-(oxetan-3-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one

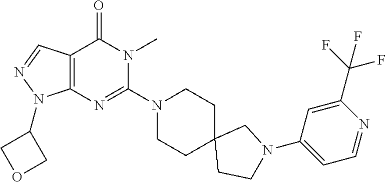 [SMILES: Cn1c(N2CCC3(CCN(c4ccnc(C(F)(F)F)c4)C3)CC2)nc2c(cnn2C2COC2)c1=O]

Step 1: tert-butyl 2-(2-(trifluoromethyl)pyridin-4-yl)-2,8-diazaspiro[4.5]decane-8-carboxylate: Followed the general procedure I using tert-butyl 2,8-diazaspiro[4.5]decane-8-carboxylate (300 mg, 1.245 mmol, 1.0 equiv.) and 4-chloro-2-(trifluoromethyl)pyridine (226 mg, 1.245 mmol, 1.0 equiv.) as the starting materials to give tert-butyl 2-(2-(trifluoromethyl)pyridin-4-yl)-2,8-diazaspiro[4.5]decane-8-carboxylate (340 mg, 60%) as a white solid. MS m/z: 386 [M+H]⁺.

Step 2: 2-(2-(trifluoromethyl)pyridin-4-yl)-2,8-diazaspiro[4.5]decane hydrochloride: Followed the general procedure B using tert-butyl 2-(2-(trifluoromethyl)pyridin-4-yl)-2,8-diazaspiro[4.5]decane-8-carboxylate (340 mg) as the starting material to give the crude product 2-(2-(trifluoromethyl)pyridin-4-yl)-2,8-diazaspiro[4.5]decane hydrochloride (340 mg). MS m/z: 286 [M+H]⁺.

Step 3: 5-methyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure I using 2-(2-(trifluoromethyl)pyridin-4-yl)-2,8-diazaspiro[4.5]decane hydrochloride (340 mg, 1.19 mmol, 1.0 equiv.) and 6-chloro-5-methyl-1-(tetrahydro-2H-pyran-2-yl)-1,5-dihydro-4H-pyrazolo[3,4-d]pyrimidin-4-one (320 mg, 1.19 mmol, 1.0 equiv.) as the starting materials to give 5-methyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (290 mg, 50%) as a white solid. MS m/z: 518 [M+H]⁺.

Step 4: 5-methyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure M using 5-methyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (290 mg, 0.56 mmol, 1.0 equiv.) as the starting material to give 5-methyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (180 mg, 55%) as a white solid. MS m/z: 434 [M+H]⁺.

Step 5: 5-methyl-1-(oxetan-3-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure K using 5-methyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (180 mg, 0.415 mmol, 1.0 equiv.) and oxetan-3-yl trifluoromethanesulfonate (256 mg, 1.24 mmol, 3.0 equiv.) as the starting materials to give 5-methyl-1-(oxetan-3-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (14.6 mg, 25%) as a white solid. ¹H NMR (300 MHz, DMSO-d₆) δ 8.21 (d, 1H), 8.08 (s, 1H), 6.88 (s, 1H), 6.69 (dd, 1H), 5.80 (p, 1H), 5.11-4.80 (m, 4H), 3.45 (d, 8H), 3.25-3.02 (m, 3H), 1.96 (t, 2H), 1.72 (t, 4H). MS m/z: 490.15 [M+H]⁺.

### Example 1060: 5-methyl-1-(oxetan-3-yl)-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one

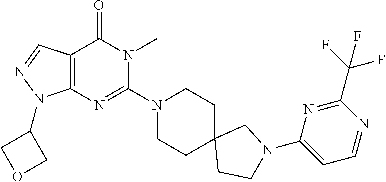 [SMILES: Cn1c(N2CCC3(CCN(c4ccnc(C(F)(F)F)n4)C3)CC2)nc2c(cnn2C2COC2)c1=O]

Step 1: tert-butyl 2-(2-(trifluoromethyl)pyrimidin-4-yl)-2,8-diazaspiro[4.5]decane-8-carboxylate: Followed the general procedure I using tert-butyl 2,8-diazaspiro[4.5]decane-8-carboxylate (300 mg, 1.25 mmol, 1.0 equiv.) and 4-chloro-2-(trifluoromethyl)pyrimidine (226 mg, 1.25 mmol, 1.0 equiv.) as the starting materials to give tert-butyl 2-(2-(trifluoromethyl)pyrimidin-4-yl)-2,8-diazaspiro[4.5]decane-8-carboxylate (360 mg, 70%) as a white solid. MS m/z: 387 [M+H]⁺

Step 2: 2-(2-(trifluoromethyl)pyrimidin-4-yl)-2,8-diazaspiro[4.5]decane: Followed the general procedure H using tert-butyl 2-(2-(trifluoromethyl)pyrimidin-4-yl)-2,8-diazaspiro[4.5]decane-8-carboxylate (360 mg) as the starting material to give the crude product 2-(2-(trifluoromethyl)pyrimidin-4-yl)-2,8-diazaspiro[4.5]decane (360 mg). MS m/z: 287 [M+H]⁺.

Step 3: 5-methyl-1-(tetrahydro-2H-pyran-2-yl)-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure I using 2-(2-(trifluoromethyl)pyrimidin-4-yl)-2,8-diazaspiro[4.5]decane (360 mg, 1.25 mmol, 1.0 equiv.) and 6-chloro-5-methyl-1-(tetrahydro-2H-pyran-2-yl)-1,5-dihydro-4H-pyrazolo[3,4-d]pyrimidin-4-one (330 mg, 1.25 mmol, 1.0 equiv.) as the starting materials to give 5-methyl-1-(tetrahydro-2H-pyran-2-yl)-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one (300 mg, 60%) as a white solid. MS m/z: 519 [M+H]⁺.

Step 4: 5-methyl-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure M using 5-methyl-1-(tetrahydro-2H-pyran-2-yl)-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one (300 mg, 0.578 mmol, 1.0 equiv.) as the starting material to give 5-methyl-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one (210 mg, 60%) as a white solid. MS m/z: 435 [M+H]⁺.

Step 5: 5-methyl-1-(oxetan-3-yl)-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure K using 5-methyl-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one (210 mg, 0.483 mmol, 1.0 equiv.) and oxetan-3-yl trifluoromethanesulfonate (298 mg, 1.45 mmol, 3.0 equiv.) as the starting materials to give 5-methyl-1-(oxetan-3-yl)-6-(2-(2-(trifluoromethyl)pyrimidin-4-yl)-2,8-diazaspiro[4.5]decan-8-yl)-1,5-dihydro-4H-pyrazolo[3,4-d]pyrimidin-4-one (33.9 mg, 35%) as a white solid. ¹H NMR (400 MHz, DMSO-d₆) δ 8.30 (d, 1H), 8.08 (s, 1H), 6.75 (dd, 1H), 5.86-5.75 (m, 1H), 5.04-4.91 (m, 4H), 3.71-3.49 (m, 2H), 3.48 (s, 1H), 3.42 (s, 3H), 3.39 (s, 1H), 3.30 (d, 1H), 3.27-3.14 (m, 3H), 2.00 (t, 1H), 1.92 (t, 1H), 1.82-1.64 (m, 4H). MS m/z: 491.15 [M+H]⁺.

### Example 1061: 8-(2-(2-(trifluoromethyl)pyridin-4-yl)-2,6-diazaspiro[3.4]octane-6-carbonyl)-7,8-dihydropyrrolo[1,2-a]pyrimidin-4(6H)-one

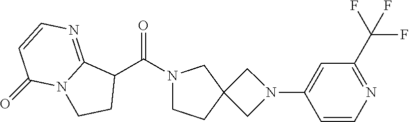 [SMILES: O=C(C1CCn2c1nccc2=O)N1CCC2(C1)CN(c1ccnc(C(F)(F)F)c1)C2]

Step 1: 8-(2-(2-(trifluoromethyl)pyridin-4-yl)-2,6-diazaspiro[3.4]octane-6-carbonyl)-7,8-dihydropyrrolo[1,2-a]pyrimidin-4(6H)-one: Followed the general procedure G using (S)-4-oxo-4,6,7,8-tetrahydropyrrolo[1,2-a]pyrimidine-8-carboxylic acid (80 mg, 0.44 mmol, 1.0 equiv.) and 2-(2-(trifluoromethyl)pyridin-4-yl)-2,6-diazaspiro[3.4]octane hydrogen chloride (130 mg, 0.44 mmol, 1.0 equiv.) as the starting materials to give 8-(2-(2-(trifluoromethyl)pyridin-4-yl)-2,6-diazaspiro[3.4]octane-6-carbonyl)-7,8-dihydropyrrolo[1,2-a]pyrimidin-4(6H)-one (76.9 mg, 41%) as a yellow solid. ¹H NMR (400 MHz, DMSO-d₆) δ 8.25 (t, J=5.4 Hz, 1H), 7.90 (d, J=6.6 Hz, 1H), 6.78-6.73 (m, 1H), 6.60-6.55 (m, 1H), 6.22 (d, J=6.6 Hz, 1H), 4.05-3.96 (m, 5H), 3.79 (t, J=9.8 Hz, 2H), 3.63-3.56 (m, 2H), 3.05 (t, J=9.6 Hz, 2H), 2.28 (d, J=6.8 Hz, 1H), 2.16-2.09 (m, 3H). MS m/z: 420.1 [M+H]⁺.

### Example 1062: 2-(difluoromethyl)-5-(6-(2-(2-(trifluoromethyl)pyridin-4-yl)-2,6-diazaspiro[3.4]octan-6-yl)pyrazin-2-yl)-1,3,4-thiadiazole

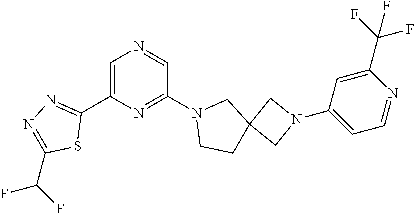 [SMILES: FC(F)c1nnc(-c2cncc(N3CCC4(CN(c5ccnc(C(F)(F)F)c5)C4)C3)n2)s1]

Step 1: 2-(difluoromethyl)-5-(6-(2-(2-(trifluoromethyl)pyridin-4-yl)-2,6-diazaspiro[3.4]octan-6-yl)pyrazin-2-yl)-1,3,4-thiadiazole: Followed the general procedure X using 6-(6-(tributylstannyl)pyrazin-2-yl)-2-(2-(trifluoromethyl)pyridin-4-yl)-2,6-diazaspiro[3.4]octane (40 mg, 64 μmol, 1.0 equiv.) and 2-bromo-5-(difluoromethyl)-1,3,4-thiadiazole (16.5 mg, 77 μmol, 1.2 equiv.) as the starting materials to give 2-(difluoromethyl)-5-(6-(2-(2-(trifluoromethyl)pyridin-4-yl)-2,6-diazaspiro[3.4]octan-6-yl)pyrazin-2-yl)-1,3,4-thiadiazole (5.8 mg, 19%) as a yellow solid. ¹H NMR (400 MHz, Chloroform-d) δ 8.87 (s, 1H), 8.33 (s, 1H), 8.05 (s, 1H), 7.05 (t, J=53.9 Hz, 1H), 6.64 (s, 1H), 6.40 (s, 1H), 4.07 (d, J=12.6 Hz, 4H), 3.86 (s, 2H), 3.71 (s, 2H), 2.42 (s, 2H). MS m/z: 470.05 [M+H]⁺.

### Example 1063: 6-(2-(2-methyl-6-(trifluoromethyl)pyrimidin-4-yl)-2,6-diazaspiro[3.4]octan-6-yl)-1-(oxetan-3-yl)-1H-pyrazolo[3,4-b]pyrazine

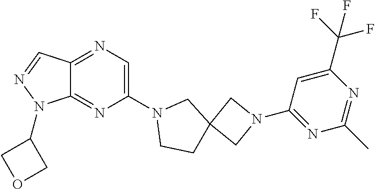 [SMILES: Cc1nc(N2CC3(CCN(c4cnc5cnn(C6COC6)c5n4)C3)C2)cc(C(F)(F)F)n1]

Step 1: tert-butyl 2-(2-methyl-6-(trifluoromethyl)pyrimidin-4-yl)-2,6-diazaspiro[3.4]octane-6-carboxylate: Followed the general procedure I using tert-butyl 2,6-diazaspiro[3.4]octane-6-carboxylate (4 g, 18.8 mmol, 1.0 equiv.) and 4-chloro-2-methyl-6-(trifluoromethyl)pyrimidine (4.07 g, 20.7 mmol, 1.1 equiv.) as the starting materials to give tert-butyl 2-(2-methyl-6-(trifluoromethyl)pyrimidin-4-yl)-2,6-diazaspiro[3.4]octane-6-carboxylate (5 g, 71%) as a white solid. MS m/z: 373 [M+H]⁺.

Step 2: 2-(2-methyl-6-(trifluoromethyl)pyrimidin-4-yl)-2,6-diazaspiro[3.4]octane hydrochloride: Followed the general procedure H using tert-butyl 2-(2-methyl-6-(trifluoromethyl)pyrimidin-4-yl)-2,6-diazaspiro[3.4]octane-6-carboxylate (5 g) as the starting material to give the crude product 2-(2-methyl-6-(trifluoromethyl)pyrimidin-4-yl)-2,6-diazaspiro[3.4]octane hydrochloride (3.4 g). MS m/z: 273 [M+H]⁺.

Step 3: 6-(2-(2-methyl-6-(trifluoromethyl)pyridimin)-2,6-diazaspiro[3.4]octan-6-yl)-1-(oxetan-3-yl)-1H-pyrazolo[3,4-b]pyrazine.

Followed the general procedure I using 2-(2-methyl-6-(trifluoromethyl)pyrimidin-4-yl)-2,6-diazaspiro[3.4]octane hydrochloride (3.4 g, 12.5 mmol, 1.0 equiv.) and 6-chloro-1-(oxetan-3-yl)-1H-pyrazolo[3,4-b]pyrazine (2.89 g, 13.7 mmol, 1.1 equiv.) as the starting materials to give the crude product. The crude product was purified by trituration with EtOH (6 mL) to afford 6-(2-(2-methyl-6-(trifluoromethyl)pyrimidin-4-yl)-2,6-diazaspiro[3.4]octan-6-yl)-1-(oxetan-3-yl)-1H-pyrazolo[3,4-b]pyrazine (2.2 g, 39%) as a white solid. ¹H NMR (400 MHz, DMSO-d₆) δ 8.19 (s, 1H), 8.08 (s, 1H), 6.66 (s, 1H), 5.94-5.82 (m, 1H), 5.07 (t, J=6.4 Hz, 2H), 4.97 (dd, J=7.8, 6.4 Hz, 2H), 4.20-4.09 (m, 4H), 3.85 (s, 2H), 3.66 (t, J=6.9 Hz, 2H), 2.43 (s, 3H), 2.30 (t, J=6.9 Hz, 2H). MS m/z: 447.1 [M+H]⁺.

### Example 1064: 1-(2,2-difluoroethyl)-3-ethyl-6-(2-(2-methyl-6-(trifluoromethyl)pyrimidin-4-yl)-2,6-diazaspiro[3.4]octan-6-yl)-1H-pyrazolo[3,4-b]pyrazine

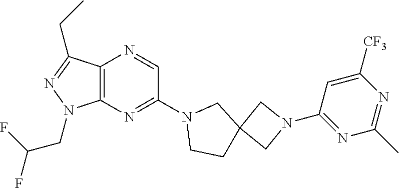 [SMILES: CCc1nn(CC(F)F)c2nc(N3CCC4(CN(c5cc(C(F)(F)F)nc(C)n5)C4)C3)cnc12]

Step 1: 6-(2-(2-methyl-6-(trifluoromethyl)pyrimidin-4-yl)-2,6-diazaspiro[3.4]octan-6-yl)-1-(tetrahydro-2H-pyran-2-yl)-3-vinyl-1H-pyrazolo[3,4-b]pyrazine: Followed the general procedure L using 3-iodo-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-b]pyrazine (240 mg, 0.400 mmol, 1.0 equiv.) and 4,4,5,5-tetramethyl-2-vinyl-1,3,2-dioxaborolane (308 mg, 2.00 mmol, 5.0 equiv.) as the starting materials to give 6-(2-(2-methyl-6-(trifluoromethyl)pyrimidin-4-yl)-2,6-diazaspiro[3.4]octan-6-yl)-1-(tetrahydro-2H-pyran-2-yl)-3-vinyl-1H-pyrazolo[3,4-b]pyrazine (140 mg, 66%) as a brown oil. MS m/z: 501 [M+H]⁺.

Step 2: 3-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-b]pyrazine: Followed the general procedure U using 6-(2-(2-methyl-6-(trifluoromethyl)pyrimidin-4-yl)-2,6-diazaspiro[3.4]octan-6-yl)-1-(tetrahydro-2H-pyran-2-yl)-3-vinyl-1H-pyrazolo[3,4-b]pyrazine (140 mg, 0.280 mmol, 1.0 equiv.) as the starting material to give 3-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-b]pyrazine (100 mg, 64%) as a brown oil. MS m/z: 503 [M+H]⁺.

Step 3: 3-ethyl-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure M using 3-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-b]pyrazine (100 mg, 0.199 mmol, 1.0 equiv.) as the starting material to give 3-ethyl-6-(2-(2-methyl-6-(trifluoromethyl)pyrimidin-4-yl)-2,6-diazaspiro[3.4]octan-6-yl)-1H-pyrazolo[3,4-b]pyrazine (65 mg, 54%) as a colorless oil. MS m/z: 419 [M+H]⁺.

Step 4: 1-(2,2-difluoroethyl)-3-ethyl-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure K using 3-ethyl-6-(2-(2-methyl-6-(trifluoromethyl)pyrimidin-4-yl)-2,6-diazaspiro[3.4]octan-6-yl)-1H-pyrazolo[3,4-b]pyrazine (60 mg, 0.143 mmol, 1.0 equiv.) and 2,2-difluoroethyl trifluoromethanesulfonate (92.1 mg, 0.429 mmol, 3.0 equiv.) as the starting materials to give 1-(2,2-difluoroethyl)-3-ethyl-6-(2-(2-methyl-6-(trifluoromethyl)pyrimidin-4-yl)-2,6-diazaspiro[3.4]octan-6-yl)-1H-pyrazolo[3,4-b]pyrazine (13.8 mg, 19.6%) as a white solid. ¹H NMR (300 MHz, DMSO-d₆) δ 8.02 (s, 1H), 6.66-6.22 (m, 2H), 4.61 (td, J=14.8, 3.9 Hz, 2H), 4.15 (q, J=9.3 Hz, 4H), 3.85 (s, 2H), 3.66 (t, J=6.9 Hz, 2H), 2.87 (q, J=7.5 Hz, 2H), 2.43 (s, 3H), 2.30 (t, J=6.9 Hz, 2H), 1.32 (t, J=7.6 Hz, 3H). MS m/z: 483.20 [M+H]⁺

### Example 1065: 3-ethyl-6-(2-(2-methyl-6-(trifluoromethyl)pyrimidin-4-yl)-2,6-diazaspiro[3.4]octan-6-yl)-1-(oxetan-3-yl)-1H-pyrazolo[3,4-b]pyrazine

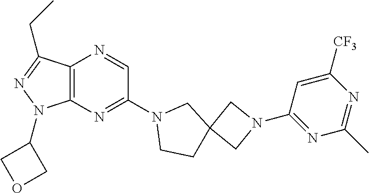 [SMILES: CCc1nn(C2COC2)c2nc(N3CCC4(CN(c5cc(C(F)(F)F)nc(C)n5)C4)C3)cnc12]

Step 1: 3-ethyl-6-(2-(2-methyl-6-(trifluoromethyl)pyrimidin-4-yl)-2,6-diazaspiro[3.4]octan-6-yl)-1-(oxetan-3-yl)-1H-pyrazolo[3,4-b]pyrazine: Followed the general procedure K using 3-ethyl-6-(2-(2-methyl-6-(trifluoromethyl)pyrimidin-4-yl)-2,6-diazaspiro[3.4]octan-6-yl)-1H-pyrazolo[3,4-b]pyrazine (160 mg, 0.382 mmol, 1.0 equiv.) and oxetan-3-yl trifluoromethanesulfonate (157 mg, 0.764 mmol, 2.0 equiv) as the starting materials to give 3-ethyl-6-(2-(2-methyl-6-(trifluoromethyl)pyrimidin-4-yl)-2,6-diazaspiro[3.4]octan-6-yl)-1-(oxetan-3-yl)-1H-pyrazolo[3,4-b]pyrazine (26.6 mg, 14.5%) as an off-white solid. ¹H NMR (400 MHz, DMSO-d₆) δ 8.00 (s, 1H), 6.66 (s, 1H), 5.87-5.74 (m, 1H), 5.07 (t, J=6.4 Hz, 2H), 4.96-4.91 (m, 2H), 4.20-4.10 (m, 4H), 3.84 (s, 2H), 3.65 (t, J=6.9 Hz, 2H), 2.90 (q, J=7.6 Hz, 3H), 2.43 (s, 3H), 2.30 (q, J=7.5, 6.9 Hz, 2H), 1.35 (t, J=7.5 Hz, 3H). MS m/z: 495.20 [M+H]⁺.

### Example 1066: 1-(2,2-difluoroethyl)-3-ethyl-6-(2-(2-methyl-6-(trifluoromethyl)pyrimidin-4-yl)-2,6-diazaspiro[3.4]octan-6-yl)-1H-pyrazolo[3,4-d]pyrimidine

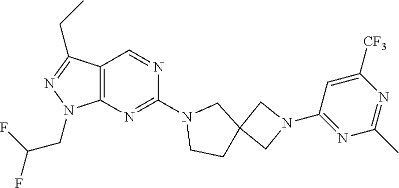 [SMILES: CCc1nn(CC(F)F)c2nc(N3CCC4(CN(c5cc(C(F)(F)F)nc(C)n5)C4)C3)ncc12]

Step 1: tert-butyl 2-(2-methyl-6-(trifluoromethyl)pyrimidin-4-yl)-2,6-diazaspiro[3.4]octane-6-carboxylate: Followed the general procedure I using tert-butyl 2,6-diazaspiro[3.4]octane-6-carboxylate hydrochloride (250 mg, 1.00 mmol, 1.0 equiv.) and 4-chloro-2-methyl-6-(trifluoromethyl)pyrimidine (217 mg, 1.10 mmol, 1.1 equiv.) as the starting materials to give tert-butyl 2-(2-methyl-6-(trifluoromethyl)pyrimidin-4-yl)-2,6-diazaspiro[3.4]octane-6-carboxylate (300 mg, 80%) as a yellow solid. MS m/z: 373 [M+H]⁺.

Step 2: 2-(2-methyl-6-(trifluoromethyl)pyrimidin-4-yl)-2,6-diazaspiro[3.4]octane: Followed the general procedure H using tert-butyl 2-(2-methyl-6-(trifluoromethyl)pyrimidin-4-yl)-2,6-diazaspiro[3.4]octane-6-carboxylate (300 mg) as the starting material to give the crude product 2-(2-methyl-6-(trifluoromethyl)pyrimidin-4-yl)-2,6-diazaspiro[3.4]octane (300 mg). MS m/z: 273 [M+H]⁺.

Step 3: 3-iodo-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-d]pyrimidine: Followed the general procedure I using 2-(2-methyl-6-(trifluoromethyl)pyrimidin-4-yl)-2,6-diazaspiro[3.4]octane (300 mg, 1.10 mmol, 1.0 equiv.) and 6-chloro-3-iodo-1-(tetrahydro-2H-pyran-2-yl)-1H-pyrazolo[3,4-d]pyrimidine (441 mg, 1.21 mmol, 1.1 equiv.) as the starting materials to give 3-iodo-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-d]pyrimidine (400 mg, 44%) as a yellow solid. MS m/z: 601 [M+H]⁺.

Step 4: 6-(2-(2-methyl-6-(trifluoromethyl)pyrimidin-4-yl)-2,6-diazaspiro[3.4]octan-6-yl)-1-(tetrahydro-2H-pyran-2-yl)-3-vinyl-1H-pyrazolo[3,4-d]pyrimidine: Followed the general procedure L using 3-iodo-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-d]pyrimidine (400 mg, 0.67 mmol, 1.0 equiv.) and 4,4,5,5-tetramethyl-2-vinyl-1,3,2-dioxaborolane (513 mg, 3.33 mmol, 5.0 equiv.) as the starting materials to give the crude product 6-(2-(2-methyl-6-(trifluoromethyl)pyrimidin-4-yl)-2,6-diazaspiro[3.4]octan-6-yl)-1-(tetrahydro-2H-pyran-2-yl)-3-vinyl-1H-pyrazolo[3,4-d]pyrimidine (300 mg) as a yellow oil. MS m/z: 501 [M+H]⁺.

Step 5: 3-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-d]pyrimidine: Followed the general procedure U using 6-(2-(2-methyl-6-(trifluoromethyl)pyrimidin-4-yl)-2,6-diazaspiro[3.4]octan-6-yl)-1-(tetrahydro-2H-pyran-2-yl)-3-vinyl-1H-pyrazolo[3,4-d]pyrimidine (300 mg, 0.60 mmol, 1.0 equiv.) as the starting material to give crude product 3-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-d]pyrimidine (300 mg) as a white solid. MS m/z: 503 [M+H]⁺.

Step 6: 3-ethyl-6-(2-(2-methyl-6-(trifluoromethyl)pyrimidin-4-yl)-2,6-diazaspiro[3.4]octan-6-yl)-1H-pyrazolo[3,4-d]pyrimidine: Followed the general procedure M using 3-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1H-pyrazolo[3,4-d]pyrimidine (300 mg, 0.59 mmol, 1.0 equiv.) as the starting material to give 3-ethyl-6-(2-(2-methyl-6-(trifluoromethyl)pyrimidin-4-yl)-2,6-diazaspiro[3.4]octan-6-yl)-1H-pyrazolo[3,4-d]pyrimidine (130 mg, 52%) as a white solid. MS m/z: 419 [M+H]⁺.

Step 7: 8-(2-(2-(trifluoromethyl)pyridin-4-yl)-2,6-diazaspiro[3.4]octane-6-carbonyl)-7,8-dihydropyrrolo[1,2-a]pyrimidin-4(6H)-one: Followed the general procedure K using 3-ethyl-6-(2-(2-methyl-6-(trifluoromethyl)pyrimidin-4-yl)-2,6-diazaspiro[3.4]octan-6-yl)-1H-pyrazolo[3,4-d]pyrimidine (65 mg, 0.16 mmol, 1.0 equiv) and 2,2-difluoroethyl trifluoromethanesulfonate (40 mg, 0.19 mmol, 1.2 equiv) as the starting materials to give 1-(2,2-difluoroethyl)-3-ethyl-6-(2-(2-methyl-6-(trifluoromethyl)pyrimidin-4-yl)-2,6-diazaspiro[3.4]octan-6-yl)-1H-pyrazolo[3,4-d]pyrimidine (17.9 mg, 24%) as a yellow solid. ¹H NMR (400 MHz, Chloroform-d₆) δ 8.85 (s, 1H), 6.35 (s, 1H), 4.61-4.53 (m, 2H), 4.18 (d, J=21.4 Hz, 4H), 4.06 (d, J=44.4 Hz, 4H), 2.96-2.85 (m, 2H), 2.59 (s, 3H), 2.41-2.26 (m, 2H), 1.39 (d, J=7.6 Hz, 3H), 1.28-1.26 (m, 1H). MS m/z: 483.2 [M+H]⁺.

### Example 1067: 3-ethyl-6-(2-(2-methyl-6-(trifluoromethyl)pyrimidin-4-yl)-2,6-diazaspiro[3.4]octan-6-yl)-1-(oxetan-3-yl)-1H-pyrazolo[3,4-d]pyrimidine

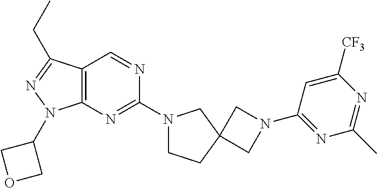 [SMILES: CCc1nn(C2COC2)c2nc(N3CCC4(CN(c5cc(C(F)(F)F)nc(C)n5)C4)C3)ncc12]

Step 1: 3-ethyl-6-(2-(2-methyl-6-(trifluoromethyl)pyrimidin-4-yl)-2,6-diazaspiro[3.4]octan-6-yl)-1-(oxetan-3-yl)-1H-pyrazolo[3,4-d]pyrimidine: Followed the general procedure K using 3-ethyl-6-(2-(2-methyl-6-(trifluoromethyl)pyrimidin-4-yl)-2,6-diazaspiro[3.4]octan-6-yl)-1H-pyrazolo[3,4-d]pyrimidine (65 mg, 0.16 mmol, 1.0 equiv.) and oxetan-3-yl trifluoromethanesulfonate (38 mg, 0.18 μmol, 1.2 equiv.) as the starting materials to give 3-ethyl-6-(2-(2-methyl-6-(trifluoromethyl)pyrimidin-4-yl)-2,6-diazaspiro[3.4]octan-6-yl)-1-(oxetan-3-yl)-1H-pyrazolo[3,4-d]pyrimidine (7.8 mg, 24%) as a yellow solid. ¹H NMR (400 MHz, Chloroform-d₆) δ 8.85 (s, 1H), 6.35 (s, 1H), 5.31-5.25 (m, 2H), 4.61-4.53 (m, 2H), 4.18 (d, J=21.4 Hz, 4H), 4.06 (d, J=44.4 Hz, 4H), 2.96-2.85 (m, 2H), 2.59 (s, 3H), 2.41-2.26 (m, 2H), 1.39 (d, J=7.6 Hz, 3H), 1.28-1.26 (m, 1H). MS m/z: 475.2 [M+H]⁺.

### Example 1068: 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-b]pyrazine

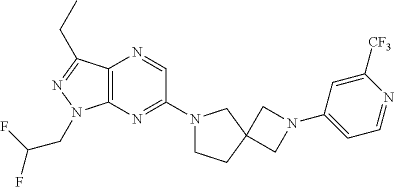 [SMILES: CCc1nn(CC(F)F)c2nc(N3CCC4(CN(c5ccnc(C(F)(F)F)c5)C4)C3)cnc12]

Step 1: 1-(2,2-difluoroethyl)-6-(2-(2-(trifluoromethyl)pyridin-4-yl)-2,6-diazaspiro[3.4]octan-6-yl)-3-vinyl-1H-pyrazolo[3,4-b]pyrazine: Followed the general procedure L using 1-(2,2-difluoroethyl)-3-iodo-6-(2-(2-(trifluoromethyl)pyridin-4-yl)-2,6-diazaspiro[3.4]octan-6-yl)-1H-pyrazolo[3,4-b]pyrazine (100 mg, 0.176 mmol, 1.0 equiv.) and 4,4,5,5-tetramethyl-2-vinyl-1,3,2-dioxaborolane (136 mg, 0.880 mmol, 5 equiv.) as the starting materials to give 1-(2,2-difluoroethyl)-6-(2-(2-(trifluoromethyl)pyridin-4-yl)-2,6-diazaspiro[3.4]octan-6-yl)-3-vinyl-1H-pyrazolo[3,4-b]pyrazine (50 mg, 60%) as a yellow solid. MS m/z: 466 [M+H]⁺

Step 2: 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-b]pyrazine: Followed the general procedure U using 1-(2,2-difluoroethyl)-6-(2-(2-(trifluoromethyl)pyridin-4-yl)-2,6-diazaspiro[3.4]octan-6-yl)-3-vinyl-1H-pyrazolo[3,4-b]pyrazine (40 mg, 0.086 mmol, 1.0 equiv.) as the starting material to give 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-b]pyrazine (18.5 mg, 42%) as a light yellow solid. 1H NMR (400 MHz, DMSO-d₆) δ 8.25 (d, J=5.7 Hz, 1H), 8.02 (s, 1H), 6.76 (d, J=2.2 Hz, 1H), 6.59-6.28 (m, 2H), 4.64-4.56 (m, 2H), 4.05 (q, J=8.4 Hz, 4H), 3.85 (s, 2H), 3.66 (t, J=6.9 Hz, 2H), 2.86 (q, J=7.6 Hz, 2H), 2.31 (t, J=7.1 Hz, 2H), 1.32 (t, J=7.5 Hz, 3H). MS m/z: 468.1 [M+H]⁺.

### Example 1069: 3-ethyl-1-(oxetan-3-yl)-6-(2-(2-(trifluoromethyl)pyridin-4-yl)-2,6-diazaspiro[3.4]octan-6-yl)-1H-pyrazolo[3,4-b]pyrazine

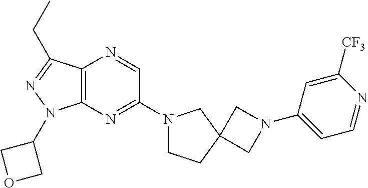 [SMILES: CCc1nn(C2COC2)c2nc(N3CCC4(CN(c5ccnc(C(F)(F)F)c5)C4)C3)cnc12]

Step 1: 2-(2-(trifluoromethyl)pyridin-4-yl)-2,6-diazaspiro[3.4]octane hydrochloride: Followed the general procedure B using tert-butyl 2-(2-(trifluoromethyl)pyridin-4-yl)-2,6-diazaspiro[3.4]octane-6-carboxylate (300 mg) as the starting material to give crude product 2-(2-(trifluoromethyl)pyridin-4-yl)-2,6-diazaspiro[3.4]octane hydrochloride (300 mg) as a white solid. MS m/z: 258 [M+H]⁺.

Step 2: 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-b]pyrazine: Followed the general procedure I using 2-(2-(trifluoromethyl)pyridin-4-yl)-2,6-diazaspiro[3.4]octane hydrochloride (300 mg, 1.16 mmol, 1.0 equiv.) and 6-chloro-3-iodo-1-(tetrahydro-2H-pyran-2-yl)-1H-pyrazolo[3,4-b]pyrazine (467 mg, 1.28 mmol, 1.1 equiv.) as the starting materials to give 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-b]pyrazine (300 mg, 46%) as a yellow solid. MS m/z: 586 [M+H]⁺.

Step 3: 1-(tetrahydro-2H-pyran-2-yl)-6-(2-(2-(trifluoromethyl)pyridin-4-yl)-2,6-diazaspiro[3.4]octan-6-yl)-3-vinyl-1H-pyrazolo[3,4-b]pyrazine: Followed the general procedure L using 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-b]pyrazine (300 mg, 0.51 mmol, 1.0 equiv.) and 4,4,5,5-tetramethyl-2-vinyl-1,3,2-dioxaborolane (395 mg, 2.55 mmol, 5.0 equiv.) as the starting materials to give 1-(tetrahydro-2H-pyran-2-yl)-6-(2-(2-(trifluoromethyl)pyridin-4-yl)-2,6-diazaspiro[3.4]octan-6-yl)-3-vinyl-1H-pyrazolo[3,4-b]pyrazine (300 mg, 83%) as a yellow oil. MS m/z: 486 [M+H]⁺.

Step 4: 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-b]pyrazine: Followed the general procedure U using 1-(tetrahydro-2H-pyran-2-yl)-6-(2-(2-(trifluoromethyl)pyridin-4-yl)-2,6-diazaspiro[3.4]octan-6-yl)-3-vinyl-1H-pyrazolo[3,4-b]pyrazine (300 mg, 0.62 mmol, 1.0 equiv.) as the starting material to give crude product 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-b]pyrazine (300 mg) as a white solid. MS m/z: 488 [M+]⁺.

Step 5: 3-ethyl-6-(2-(2-(trifluoromethyl)pyridin-4-yl)-2,6-diazaspiro[3.4]octan-6-yl)-1H-pyrazolo[3,4-b]pyrazine: Followed the general procedure M using 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-b]pyrazine (300 mg, 0.62 mmol, 1.0 equiv.) as the starting material to give 3-ethyl-6-(2-(2-(trifluoromethyl)pyridin-4-yl)-2,6-diazaspiro[3.4]octan-6-yl)-1H-pyrazolo[3,4-b]pyrazine (130 mg, 28%) as a white solid. MS m/z: 404 [M+H]⁺.

Step 6: 3-ethyl-1-(oxetan-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure K using 3-ethyl-6-(2-(2-(trifluoromethyl)pyridin-4-yl)-2,6-diazaspiro[3.4]octan-6-yl)-1H-pyrazolo[3,4-b]pyrazine (70 mg, 0.17 mmol, 1.0 equiv.) and oxetan-3-yl trifluoromethanesulfonate (43 mg, 0.21 mmol, 1.2 equiv.) as the starting materials to give 3-ethyl-1-(oxetan-3-yl)-6-(2-(2-(trifluoromethyl)pyridin-4-yl)-2,6-diazaspiro[3.4]octan-6-yl)-1H-pyrazolo[3,4-b]pyrazine (13 mg, 16%) as a yellow solid. ¹H NMR (400 MHz, DMSO-d₆) δ 8.25 (d, J=5.6 Hz, 1H), 8.01 (s, 1H), 6.76 (d, J=2.2 Hz, 1H), 6.61-6.55 (m, 1H), 5.86-5.75 (m, 1H), 5.07 (t, J=6.4 Hz, 2H), 4.97-4.89 (m, 2H), 4.10-3.99 (m, 4H), 3.84 (s, 2H), 3.65 (t, J=6.8 Hz, 2H), 2.95-2.79 (m, 2H), 2.30 (t, J=6.8 Hz, 2H), 1.35 (t, J=7.6 Hz, 3H). MS m/z: 460.1 [M+H]⁺.

### Example 1070: 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine

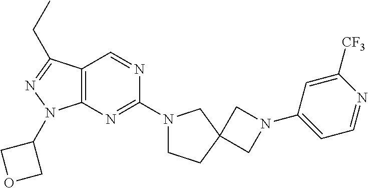 [SMILES: CCc1nn(C2COC2)c2nc(N3CCC4(CN(c5ccnc(C(F)(F)F)c5)C4)C3)ncc12]

Step 1: 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine: Followed the general procedure I using 2-(2-(trifluoromethyl)pyridin-4-yl)-2,6-diazaspiro[3.4]octane hydrogen chloride (300 mg, 1.12 mmol, 1.0 equiv.) and 6-chloro-3-iodo-1-(tetrahydro-2H-pyran-2-yl)-1H-pyrazolo[3,4-d]pyrimidine (467 mg, 1.28 mmol, 1.1 equiv.) as the starting materials to give 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (300 mg, 46%) as a yellow solid. MS m/z: 586 [M+H]⁺.

Step 2: 1-(tetrahydro-2H-pyran-2-yl)-6-(2-(2-(trifluoromethyl)pyridin-4-yl)-2,6-diazaspiro[3.4]octan-6-yl)-3-vinyl-1H-pyrazolo[3,4-d]pyrimidine: Followed the general procedure L using 3-iodo-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (300 mg, 0.51 mmol, 1.0 equiv.) and 4,4,5,5-tetramethyl-2-vinyl-1,3,2-dioxaborolane (395 mg, 2.56 mmol, 5.0 equiv.) as the starting materials to give 1-(tetrahydro-2H-pyran-2-yl)-6-(2-(2-(trifluoromethyl)pyridin-4-yl)-2,6-diazaspiro[3.4]octan-6-yl)-3-vinyl-1H-pyrazolo[3,4-d]pyrimidine (300 mg, 46%) as a yellow oil. MS m/z: 486 [M+H]⁺.

Step 3: 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine: Followed the general procedure U using 1-(tetrahydro-2H-pyran-2-yl)-6-(2-(2-(trifluoromethyl)pyridin-4-yl)-2,6-diazaspiro[3.4]octan-6-yl)-3-vinyl-1H-pyrazolo[3,4-d]pyrimidin (300 mg, 0.62 mmol, 1.0 equiv.) as the starting material to give crude product 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (300 mg) as a white solid. MS m/z: 488 [M+H]⁺.

Step 4: 3-ethyl-6-(2-(2-(trifluoromethyl)pyridin-4-yl)-2,6-diazaspiro[3.4]octan-6-yl)-1H-pyrazolo[3,4-d]pyrimidine: Followed the general procedure M using 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (300 mg, 616 μmol, 1.0 equiv.) as the starting material to give 3-ethyl-6-(2-(2-(trifluoromethyl)pyridin-4-yl)-2,6-diazaspiro[3.4]octan-6-yl)-1H-pyrazolo[3,4-d]pyrimidine (130 mg, 28%) as a white solid. MS m/z: 404 [M+H]⁺.

Step 5: 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine: Followed the general procedure K using 3-ethyl-6-(2-(2-(trifluoromethyl)pyridin-4-yl)-2,6-diazaspiro[3.4]octan-6-yl)-1H-pyrazolo[3,4-d]pyrimidine (70 mg, 0.17 mmol, 1.0 equiv.) and oxetan-3-yl trifluoromethanesulfonate (43 mg, 0.21 mmol, 1.2 equiv.) as the starting materials to give 3-ethyl-1-(tetrahydro-2H-pyran-2-yl)-6-(2-(2-(trifluoromethyl)pyridin-4-yl)-2,6-diazaspiro[3.4]octan-6-yl)-1H-pyrazolo[3,4-d]pyrimidine (4.4 mg, 5%) as a white solid. ¹H NMR (400 MHz, DMSO-d₆) δ 8.25 (d, J=5.6 Hz, 1H), 8.01 (s, 1H), 6.76 (d, J=2.4 Hz, 1H), 6.61-6.55 (m, 1H), 5.86-5.75 (m, 1H), 5.07 (t, J=6.4 Hz, 2H), 4.97-4.89 (m, 2H), 4.10-3.99 (m, 4H), 3.84 (s, 2H), 3.65 (t, J=6.8 Hz, 2H), 2.95-2.79 (m, 2H), 2.30 (t, J=6.8 Hz, 2H), 1.35 (t, J=7.6 Hz, 3H). MS m/z: 460.15 [M+H]⁺.

### Example 1071: 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-d]pyrimidine

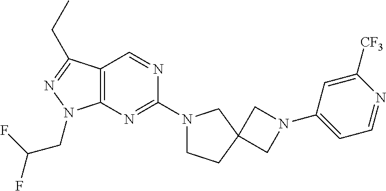 [SMILES: CCc1nn(CC(F)F)c2nc(N3CCC4(CN(c5ccnc(C(F)(F)F)c5)C4)C3)ncc12]

Step 1: 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-d]pyrimidine: Followed the general procedure K using 3-ethyl-6-(2-(2-(trifluoromethyl)pyridin-4-yl)-2,6-diazaspiro[3.4]octan-6-yl)-1H-pyrazolo[3,4-d]pyrimidine (100 mg, 0.25 mmol, 1.0 equiv.) and 2,2-difluoroethyl trifluoromethanesulfonate (53 mg, 0.25 mmol, 1.0 equiv.) as the starting materials to give 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-d]pyrimidine (11.4 mg, 10%) as a white solid. ¹H NMR (400 MHz, Chloroform-d₆) δ 8.74 (s, 1H), 8.32 (d, J=5.4 Hz, 1H), 6.63 (d, J=2.0 Hz, 1H), 6.43-6.06 (m, 2H), 4.56 (td, J=13.2, 4.5 Hz, 2H), 4.10-3.99 (m, 4H), 3.93 (s, 2H), 3.79 (s, 2H), 2.95-2.85 (m, 2H), 2.33 (t, J=6.8 Hz, 2H), 1.38 (t, J=7.6 Hz, 3H). MS m/z: 468.1 [M+H]⁺.

### Example 1072: 1-(2,2-difluoroethyl)-6-(2-(2-(trifluoromethyl)pyridin-4-yl)-2,6-diazaspiro[3.4]octan-6-yl)-1H-pyrazolo[4,3-c]pyridine

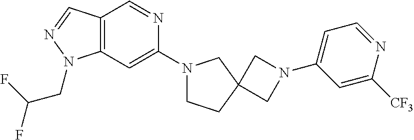 [SMILES: FC(F)Cn1ncc2cnc(N3CCC4(CN(c5ccnc(C(F)(F)F)c5)C4)C3)cc21]

Step 1: 6-chloro-1-(2,2-difluoroethyl)-1H-pyrazolo[4,3-c]pyridine: Followed the general procedure K using 6-chloro-1H-pyrazolo[4,3-c]pyridine (300 mg, 1.95 mmol, 1.0 equiv.) and 2,2-difluoroethyl trifluoromethanesulfonate (627 mg, 2.93 mmol, 1.5 equiv.) as the starting materials to give 6-chloro-1-(2,2-difluoroethyl)-1H-pyrazolo[4,3-c]pyridine (310 mg, 73%) as a white solid. MS m/z: 218 [M+H]⁺.

Step 2: 1-(2,2-difluoroethyl)-6-(2-(2-(trifluoromethyl)pyridin-4-yl)-2,6-diazaspiro[3.4]octan-6-yl)-1H-pyrazolo[4,3-c]pyridine: Followed the general procedure Y using 6-chloro-1-(2,2-difluoroethyl)-1H-pyrazolo[4,3-c]pyridine (50 mg, 0.23 mmol, 1.2 equiv.) and 2-(2-(trifluoromethyl)pyridin-4-yl)-2,6-diazaspiro[3.4]octane hydrochloride (49.3 mg, 0.192 mmol, 1 equiv.) as the starting materials, Pd₂(dba)₃ (17.5 mg, 0.019 mmol, 0.1 equiv.) and BINAP (12.4 mg, 0.019 mmol, 0.1 equiv.) as the catalyst, as the ligand to give 1-(2,2-difluoroethyl)-6-(2-(2-(trifluoromethyl)pyridin-4-yl)-2,6-diazaspiro[3.4]octan-6-yl)-1H-pyrazolo[4,3-c]pyridine (60.2 mg, 69%) as a white solid. ¹H NMR (400 MHz, DMSO-d₆) δ 8.67 (s, 1H), 8.25 (d, J=5.7 Hz, 1H), 8.08 (s, 1H), 6.77 (d, J=2.3 Hz, 1H), 6.59 (d, J=6.0 Hz, 1H), 6.54-6.19 (m, 2H), 4.76 (td, J=13.4 Hz, 3.4 Hz, 2H), 4.11-3.99 (m, 4H), 3.71 (s, 2H), 3.52 (t, J=6.8 Hz, 2H), 2.29 (t, J=6.8 Hz, 2H). MS m/z: 439.15 [M+H]⁺.

### Example 1073: 1-(2,2-difluoroethyl)-6-(2-(2-(trifluoromethyl)pyridin-4-yl)-2,6-diazaspiro[3.4]octan-6-yl)-1H-pyrazolo[3,4-b]pyridine

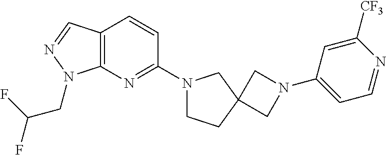 [SMILES: FC(F)Cn1ncc2ccc(N3CCC4(CN(c5ccnc(C(F)(F)F)c5)C4)C3)nc21]

Step 1: 6-chloro-1-(2,2-difluoroethyl)-1H-pyrazolo[3,4-b]pyridine: Followed the general procedure K using 6-chloro-1H-pyrazolo[3,4-b]pyridine (200 mg, 1.30 mmol, 1.0 equiv.) and 2,2-difluoroethyl trifluoromethanesulfonate (334 mg, 1.56 mmol, 1.2 equiv.) as the starting materials to give 6-chloro-1-(2,2-difluoroethyl)-1H-pyrazolo[3,4-b]pyridine (80 mg, 28%) as a white solid. MS m/z: 218 [M+H]⁺.

Step 2: 1-(2,2-difluoroethyl)-6-(2-(2-(trifluoromethyl)pyridin-4-yl)-2,6-diazaspiro[3.4]octan-6-yl)-1H-pyrazolo[3,4-b]pyridine: Followed the general procedure I using 2-(2-(trifluoromethyl)pyridin-4-yl)-2,6-diazaspiro[3.4]octane (113 mg, 0.442 mmol, 1.2 equiv.) and 6-chloro-1-(2,2-difluoroethyl)pyrazolo[3,4-b]pyridine hydrochloride (80 mg, 0.368 mmol, 1.0 equiv.) as the starting materials to give 1-(2,2-difluoroethyl)-6-(2-(2-(trifluoromethyl)pyridin-4-yl)-2,6-diazaspiro[3.4]octan-6-yl)-1H-pyrazolo[3,4-b]pyridine (22 mg, 11%) as a white solid. ¹H NMR (400 MHz, DMSO-d₆) δ 8.25 (d, J=5.7 Hz, 1H), 7.93-7.85 (m, 2H), 6.76 (d, J=2.3 Hz, 1H), 6.59 (dt, J=5.7, 3.1 Hz, 1H), 6.52-6.42 (m, 2H), 4.68 (td, J=14.7, 4.1 Hz, 2H), 4.04 (q, J=8.3 Hz, 4H), 3.79 (s, 2H), 3.59 (t, J=6.9 Hz, 2H), 2.29 (t, J=6.9 Hz, 2H). MS m/z: 439.10 [M+H]⁺.

### Example 1074: 3-(2,2-difluoroethyl)-1-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one; Example 1075: 1-(2,2-difluoroethyl)-3-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one

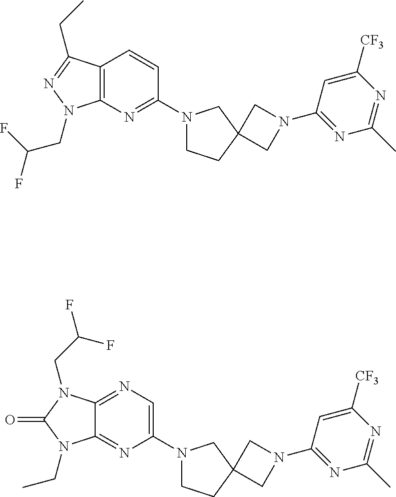 [SMILES: CCc1nn(CC(F)F)c2nc(N3CCC4(CN(c5cc(C(F)(F)F)nc(C)n5)C4)C3)ccc12 ; CCn1c(=O)n(CC(F)F)c2ncc(N3CCC4(CN(c5cc(C(F)(F)F)nc(C)n5)C4)C3)nc21]

Step 1: 3-(2,2-difluoroethyl)-1-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one; 1-(2,2-difluoroethyl)-3-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one: Followed the general procedure Y using 5-bromo-3-(2,2-difluoroethyl)-1-ethyl-1,3-dihydro-2H-imidazo[4,5-b]pyrazin-2-one and 5-bromo-1-(2,2-difluoroethyl)-3-ethyl-1,3-dihydro-2H-imidazo[4,5-b]pyrazin-2-one two isomers (55 mg, 0.179 mmol, 1.0 equiv.) and 2-(2-methyl-6-(trifluoromethyl)pyrimidin-4-yl)-2,6-diazaspiro[3.4]octane (48.8 mg, 0.179 mmol, 1.0 equiv.) as the starting materials to give the crude product. The crude product was purified by Prep-HPLC to afford 3-(2,2-difluoroethyl)-1-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one (19.3 mg, 22%) as a white solid, and 1-(2,2-difluoroethyl)-3-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one (10.9 mg, 12%) as a white solid.

3-(2,2-difluoroethyl)-1-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one ¹H NMR (300 MHz, Chloroform-d) δ 7.20 (d, J=1.4 Hz, 1H), 6.41-6.00 (m, 2H), 4.31-4.13 (m, 6H), 4.03-3.96 (m, 2H), 3.72 (s, 2H), 3.58-3.52 (m, 2H), 2.59 (d, J=1.4 Hz, 3H), 2.34 (t, J=6.4 Hz, 2H), 1.41-1.35 (m, 3H). MS m/z: 499.15 [M+H]⁺.

1-(2,2-difluoroethyl)-3-ethyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one ¹H NMR (300 MHz, Chloroform-d) δ 7.16 (s, 1H), 6.37-5.97 (m, 2H), 4.32-4.17 (m, 6H), 3.98 (q, J=7.2 Hz, 2H), 3.74 (s, 2H), 3.56 (t, J=6.8 Hz, 2H), 2.63 (s, 3H), 2.35 (t, J=6.8 Hz, 2H), 1.39 (t, J=7.2 Hz, 3H). MS m/z: 499.15 [M+H]⁺.

### Example 1076: 1-(2,2-difluoropropyl)-6-(2-(6-methyl-2-(trifluoromethyl)pyrimidin-4-yl)-2,6-diazaspiro[3.4]octan-6-yl)-1H-pyrazolo[3,4-d]pyrimidine

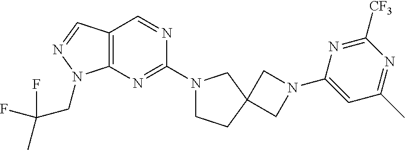 [SMILES: Cc1cc(N2CC3(CCN(c4ncc5cnn(CC(C)(F)F)c5n4)C3)C2)nc(C(F)(F)F)n1]

Step 1: 6-chloro-1-(2,2-difluoropropyl)-1H-pyrazolo[3,4-d]pyrimidine: Followed the general procedure K using 6-chloro-1H-pyrazolo[3,4-d]pyrimidine (100 mg, 647 μmol, 1.0 equiv.) and 2,2-difluoropropyl trifluoromethanesulfonate (177 mg, 776 μmol, 1.2 equiv.) as the starting materials to give 6-chloro-1-(2,2-difluoropropyl)-1H-pyrazolo[3,4-d]pyrimidine (130 mg, 86%) as a white solid. MS m/z: 233 [M+H]⁺

Step 2: 1-(2,2-difluoropropyl)-6-(2-(6-methyl-2-(trifluoromethyl)pyrimidin-4-yl)-2,6-diazaspiro[3.4]octan-6-yl)-1H-pyrazolo[3,4-d]pyrimidine: Followed the general procedure I using 6-chloro-1-(2,2-difluoropropyl)-1H-pyrazolo[3,4-d]pyrimidine (100 mg, 430 μmol, 1.0 equiv.) and 2-(6-methyl-2-(trifluoromethyl)pyrimidin-4-yl)-2,6-diazaspiro[3.4]octane (128 mg, 473 μmol, 1.1 equiv.) as the starting materials to give 1-(2,2-difluoropropyl)-6-(2-(6-methyl-2-(trifluoromethyl)pyrimidin-4-yl)-2,6-diazaspiro[3.4]octan-6-yl)-1H-pyrazolo[3,4-d]pyrimidine (40.1 mg, 20%) as a white solid. ¹H NMR (400 MHz, DMSO-d₆) δ 8.92 (s, 1H), 8.08 (s, 1H), 6.49 (s, 1H), 4.67 (t, J=12.8 Hz, 2H), 4.17-4.01 (m, 4H), 3.84 (s, 2H), 3.67 (s, 2H), 2.34 (s, 3H), 2.27 (t, J=6.9 Hz, 2H), 1.66 (t, J=19.2 Hz, 3H). MS m/z: 469.30 [M+H]⁺.

### Example 1077: 3-(2,2-difluoroethyl)-1-methyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one; Example 1078: 1-(2,2-difluoroethyl)-3-methyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one

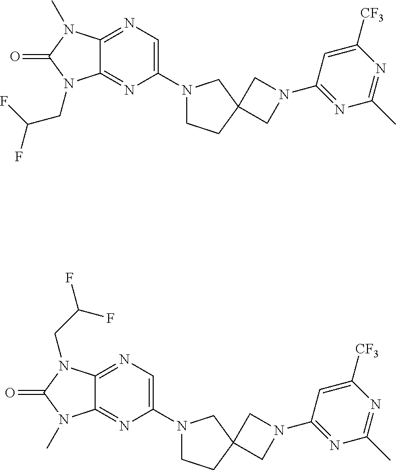 [SMILES: Cc1nc(N2CC3(CCN(c4cnc5c(n4)n(CC(F)F)c(=O)n5C)C3)C2)cc(C(F)(F)F)n1 ; Cc1nc(N2CC3(CCN(c4cnc5c(n4)n(C)c(=O)n5CC(F)F)C3)C2)cc(C(F)(F)F)n1]

Step 1: 3-(2,2-difluoroethyl)-1-methyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one; 1-(2,2-difluoroethyl)-3-methyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one: Followed the general procedure Y using 5-bromo-3-(2,2-difluoroethyl)-1-methyl-1,3-dihydro-2H-imidazo[4,5-b]pyrazin-2-one and 5-bromo-1-(2,2-difluoroethyl)-3-methyl-1,3-dihydro-2H-imidazo[4,5-b]pyrazin-2-one two isomers (85.8 mg, 0.294 mmol, 1.0 equiv.) and 2-(2-methyl-6-(trifluoromethyl)pyrimidin-4-yl)-2,6-diazaspiro[3.4]octane (80 mg, 0.294 mmol, 1.0 equiv.) as the starting materials to give the crude product. The crude product was purified by Prep-HPLC to afford 3-(2,2-difluoroethyl)-1-methyl-5-(2-(2-methyl-6-(trifluoromethyl) pyrimidin-4-yl)-2,6-diazaspiro[3.4]octan-6-yl)-1,3-dihydro-2H-imidazo[4,5-b]pyrazin-2-one (26.3 mg, 31%) as a white solid, and 1-(2,2-difluoroethyl)-3-methyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one (12.4 mg, 23%) as a white solid.

3-(2,2-difluoroethyl)-1-methyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one ¹H NMR (300 MHz, Chloroform-d) δ 7.20 (s, 1H), 6.40-6.00 (m, 2H), 4.32-4.17 (m, 6H), 3.73 (s, 2H), 3.57 (t, J=6.8 Hz, 2H), 3.46 (s, 3H), 2.61 (s, 3H), 2.35 (t, J=6.9 Hz, 2H). MS m/z: 485.1 [M+H]⁺.

1-(2,2-difluoroethyl)-3-methyl-5-(2-(2-methyl-6-(trifluoromethyl)pyrimidin-4-yl)-2,6-diazaspiro[3.4]octan-6-yl)-1,3-dihydro-2H-imidazo[4,5-b]pyrazin-2-one ¹H NMR (300 MHz, Chloroform-d) δ 7.16 (s, 1H), 6.37-5.96 (m, 2H), 4.33-4.15 (m, 6H), 3.74 (s, 2H), 3.57 (t, J=6.9 Hz, 2H), 3.43 (s, 3H), 2.59 (s, 3H), 2.34 (t, J=6.9 Hz, 2H). MS m/z: 485.1 [M+H]⁺.

### Example 1079: (S)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-d]pyrimidine; Example 1080: (R)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-d]

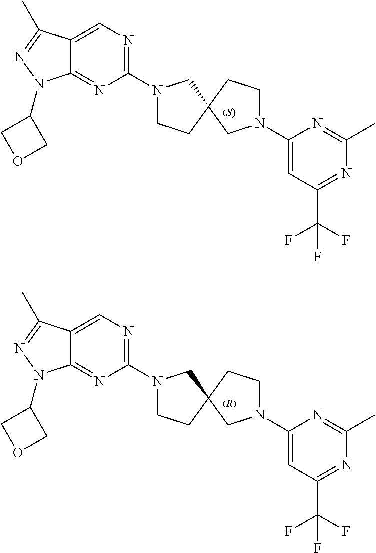

Step 1: 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-d]pyrimidine. The crude 1-(2,2-difluoroethyl)-3-ethyl-6-(2-(2-(trifluoromethyl)pyridin-4-yl)-2,6-diazaspiro[3.4]octan-6-yl)-1H-pyrazolo[3,4-d]pyrimidine was purified by Prep-HPLC with the following conditions (Column: NB_CHIRALPAK AD-H, 3*25 cm, 5 m; Mobile Phase A: CO₂, Mobile Phase B: IPA; Flow rate: 100 mL/min; Gradient: isocratic 25% B; Column Temperature (° C.): 35; Back Pressure (bar): 100; Wave Length: 246/210 nm; RT1(min): 4.87; RT2(min): 6.7; Sample Solvent: MeOH-HPLC; Injection Volume: 3 mL; Number Of Runs: 3) to afford (R)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-d]pyrimidine (32.2 mg, 22.23%) as a white solid, and (S)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-d]pyrimidine (30.2 mg, 22.23%) as a white solid.

(S)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-d]pyrimidine ¹H NMR (400 MHz, DMSO-d₆) δ 8.86 (s, 1H), 6.73 (d, J=4.8 Hz, 1H), 5.79 (t, J=7.2 Hz, 1H), 4.96 (d, J=31.8 Hz, 4H), 3.74-3.49 (m, 8H), 2.43 (d, J=14.4 Hz, 3H), 2.10-1.98 (m, 4H), MS m/z: 475.3 [M+H]⁺.

(R)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-d]pyrimidine ¹H NMR (400 MHz, DMSO-d₆) δ 8.86 (s, 1H), 6.73 (d, J=5.0 Hz, 1H), 5.79 (p, J=7.1 Hz, 1H), 5.09-4.85 (m, 4H), 3.76-3.45 (m, 8H), 2.41 (s, 3H), 2.10-1.97 (m, 4H), MS m/z: 475.2 [M+H]⁺.

### Example 1081: 9-(2,2-difluoroethyl)-7-ethyl-2-(2-(2-methyl-6-(trifluoromethyl)pyrimidin-4-yl)-2,6-diazaspiro[3.4]octan-6-yl)-7,9-dihydro-8H-purin-8-one

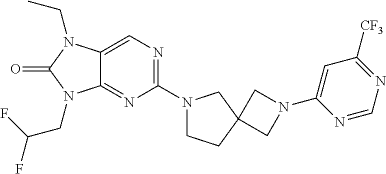 [SMILES: CCn1c(=O)n(CC(F)F)c2nc(N3CCC4(CN(c5cc(C(F)(F)F)ncn5)C4)C3)ncc21]

Step 1: 9-(2,2-difluoroethyl)-7-ethyl-2-(2-(2-methyl-6-(trifluoromethyl)pyrimidin-4-yl)-2,6-diazaspiro[3.4]octan-6-yl)-7,9-dihydro-8H-purin-8-one: Followed the general procedure I using 2-chloro-9-(2,2-difluoroethyl)-7-ethyl-7,9-dihydro-8H-purin-8-one (70 mg, 0.267 mmol, 1.0 equiv.) and 2-(2-methyl-6-(trifluoromethyl)pyrimidin-4-yl)-2,6-diazaspiro[3.4]octane (87 mg, 0.320 mmol, 1.2 equiv.) as the starting materials to give 9-(2,2-difluoroethyl)-7-ethyl-2-(2-(2-methyl-6-(trifluoromethyl)pyrimidin-4-yl)-2,6-diazaspiro[3.4]octan-6-yl)-7,9-dihydro-8H-purin-8-one (44 mg, 33%) as a white solid. ¹H NMR (400 MHz, DMSO-d₆) δ 8.15 (s, 1H), 6.64 (s, 1H), 6.59-6.19 (m, 1H), 4.24-4.01 (m, 6H), 3.83 (t, J=7.2 Hz, 2H), 3.80-3.66 (m, 2H), 3.61-3.50 (m, 2H), 2.42 (s, 3H), 2.22 (t, J=6.9 Hz, 2H), 1.22 (t, J=7.1 Hz, 3H). MS m/z: 499.1 [M+H]⁺.

### Example 1082: 9-(2,2-difluoroethyl)-7-methyl-2-(2-(2-methyl-6-(trifluoromethyl)pyrimidin-4-yl)-2,6-diazaspiro[3.4]octan-6-yl)-7,9-dihydro-8H-purin-8-one

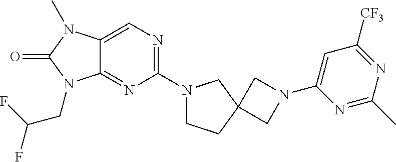 [SMILES: Cc1nc(N2CC3(CCN(c4ncc5c(n4)n(CC(F)F)c(=O)n5C)C3)C2)cc(C(F)(F)F)n1]

Step 1: 9-(2,2-difluoroethyl)-7-methyl-2-(2-(2-methyl-6-(trifluoromethyl)pyrimidin-4-yl)-2,6-diazaspiro[3.4]octan-6-yl)-7,9-dihydro-8H-purin-8-one: Followed the general procedure I using 2-chloro-9-(2,2-difluoroethyl)-7-methyl-7,9-dihydro-8H-purin-8-one (80 mg, 0.322 mmol, 1.0 equiv.) and 2-(2-methyl-6-(trifluoromethyl)pyrimidin-4-yl)-2,6-diazaspiro[3.4]octane (105 mg, 0.386 mmol, 1.2 equiv.) as the starting materials to give 9-(2,2-difluoroethyl)-7-methyl-2-(2-(2-methyl-6-(trifluoromethyl)pyrimidin-4-yl)-2,6-diazaspiro[3.4]octan-6-yl)-7,9-dihydro-8H-purin-8-one (32.5 mg, 20%) as a white solid. ¹H NMR (400 MHz, DMSO-d₆) δ 8.08 (s, 1H), 6.64 (s, 1H), 6.43-6.23 (m, 1H), 4.73-3.91 (m, 6H), 3.73 (d, J=4.0 Hz, 2H), 3.55 (t, J=6.8 Hz, 2H), 3.30 (s, 3H), 2.48 (s, 3H), 2.42 (s, 2H). MS m/z: 485.1 [M+H]⁺.

### Example 1083: 9-(2,2-difluoroethyl)-7-ethyl-2-(2-(2-methyl-4-(trifluoromethyl)pyrimidin-5-yl)-2,6-diazaspiro[3.4]octan-6-yl)-7,9-dihydro-8H-purin-8-one

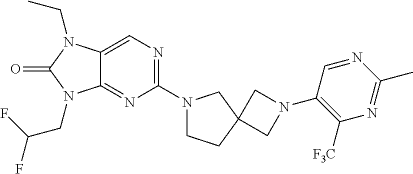 [SMILES: CCn1c(=O)n(CC(F)F)c2nc(N3CCC4(C3)CN(c3cnc(C)nc3C(F)(F)F)C4)ncc21]

Step 1: 9-(2,2-difluoroethyl)-7-ethyl-2-(2-(2-methyl-4-(trifluoromethyl)pyrimidin-5-yl)-2,6-diazaspiro[3.4]octan-6-yl)-7,9-dihydro-8H-purin-8-one: Followed the general procedure I using 2-chloro-9-(2,2-difluoroethyl)-7-ethyl-7,9-dihydro-8H-purin-8-one (50 mg, 0.19 mmol, 1.0 equiv.) and 2-(2-methyl-4-(trifluoromethyl)pyrimidin-5-yl)-2,6-diazaspiro[3.4]octane (67.4 mg, 0.247 mmol, 1.3 equiv.) as the starting materials to give 9-(2,2-difluoroethyl)-7-ethyl-2-(2-(2-methyl-4-(trifluoromethyl)pyrimidin-5-yl)-2,6-diazaspiro[3.4]octan-6-yl)-7,9-dihydro-8H-purin-8-one (4.7 mg, 5%) as a white solid. ¹H NMR (400 MHz, DMSO-d₆) δ 8.30 (s, 1H), 8.15 (s, 1H), 6.73-6.16 (m, 1H), 4.25-4.12 (m, 2H), 4.08 (q, J=7.9 Hz, 4H), 3.82 (q, J=7.2 Hz, 2H), 3.72 (s, 2H), 3.55 (t, J=6.9 Hz, 2H), 2.54 (s, 3H), 2.24 (t, J=6.8 Hz, 2H), 1.21 (t, J=7.2 Hz, 3H). MS m/z: 499.15 [M+H]⁺.

### Example 1084: 1-(2,2-difluoroethyl)-5-methyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one

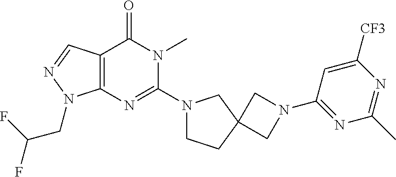 [SMILES: Cc1nc(N2CC3(CCN(c4nc5c(cnn5CC(F)F)c(=O)n4C)C3)C2)cc(C(F)(F)F)n1]

Step 1: 2-(2-methyl-6-(trifluoromethyl)pyrimidin-4-yl)-2,6-diazaspiro[3.4]octane: Followed the general procedure H using tert-butyl 2-(2-methyl-6-(trifluoromethyl)pyrimidin-4-yl)-2,6-diazaspiro[3.4]octane-6-carboxylate (150 mg) as the starting material to give the crude product 2-(2-methyl-6-(trifluoromethyl)pyrimidin-4-yl)-2,6-diazaspiro[3.4]octane (150 mg). MS m/z: 273 [M+H]⁺.

Step 2: 5-methyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure I using 2-(2-methyl-6-(trifluoromethyl)pyrimidin-4-yl)-2,6-diazaspiro[3.4]octane (150 mg, 0.55 mmol, 1.0 equiv.) and 6-chloro-5-methyl-1-(tetrahydro-2H-pyran-2-yl)-1,5-dihydro-4H-pyrazolo[3,4-d]pyrimidin-4-one (147 mg, 0.550 mmol, 1.0 equiv.) as the starting materials to give 5-methyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (125 mg, 50%) as a white solid. MS m/z: 505 [M+H]⁺.

Step 3: 5-methyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure M using 5-methyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (125 mg, 0.248 mmol, 1.0 equiv.) as the starting material to give 5-methyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (80.0 mg, 50%) as a white solid. MS m/z: 421 [M+H]⁺.

Step 4: 1-(2,2-difluoroethyl)-5-methyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure K using 5-methyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (80.0 mg, 0.19 mmol, 1.0 equiv.) and 2,2-difluoroethyl trifluoromethanesulfonate (122 mg, 0.57 mmol, 3.0 equiv.) as the starting materials to give 1-(2,2-difluoroethyl)-5-methyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (47.2 mg, 50%) as a white solid. ¹H NMR (400 MHz, DMSO-d₆) δ 7.95 (s, 1H), 6.64 (s, 1H), 6.43 (tt, 1H), 4.59 (td, 2H), 4.18-3.98 (m, 4H), 3.81 (d, 2H), 3.65 (t, 2H), 3.44 (s, 3H), 2.42 (s, 3H), 2.19 (t, 2H). MS m/z: 485.1 [M+H]⁺.

### Example 1085: 3-(2,2-difluoroethyl)-1-ethyl-5-(2-(4-methyl-2-(trifluoromethyl)pyrimidin-5-yl)-2,8-diazaspiro[4.5]decan-8-yl)-1,3-dihydro-2H-imidazo[4,5-b]pyrazin-2-one

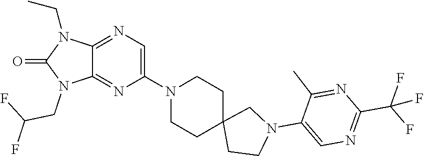 [SMILES: CCn1c(=O)n(CC(F)F)c2nc(N3CCC4(CC3)CCN(c3cnc(C(F)(F)F)nc3C)C4)cnc21]

Step 1: 3-(2,2-difluoroethyl)-1-ethyl-5-(2-(4-methyl-2-(trifluoromethyl)pyrimidin-5-yl)-2,8-diazaspiro[4.5]decan-8-yl)-1,3-dihydro-2H-imidazo[4,5-b]pyrazin-2-one: Followed the general procedure Y using 2-(4-methyl-2-(trifluoromethyl)pyrimidin-5-yl)-2,8-diazaspiro[4.5]decane hydrochloride (40.0 mg, 0.133 mmol, 1.0 equiv.) and 5-bromo-3-(2,2-difluoroethyl)-1-ethyl-1,3-dihydro-2H-imidazo[4,5-b]pyrazin-2-one (40.8 mg, 0.133 mmol, 1.0 equiv.) as the starting materials to give 3-(2,2-difluoroethyl)-1-ethyl-5-(2-(4-methyl-2-(trifluoromethyl)pyrimidin-5-yl)-2,8-diazaspiro[4.5]decan-8-yl)-1,3-dihydro-2H-imidazo[4,5-b]pyrazin-2-one (20 mg, 29%) as a white solid. ¹H NMR (400 MHz, DMSO-d₆) δ 8.14 (s, 1H), 7.65 (s, 1H), 6.37 (tt, 1H), 4.23 (td, 2H), 3.86 (q, 2H), 3.63-3.47 (m, 6H), 3.39 (s, 2H), 2.64 (s, 3H), 1.88 (t, 2H), 1.79-1.55 (m, 4H), 1.27 (t, 3H). MS m/z: 527.35 [M+H]⁺.

### Example 1086: 1-(2,2-difluoroethyl)-5-methyl-6-(2-(4-methyl-2-(trifluoromethyl)pyrimidin-5-yl)-2,8-diazaspiro[4.5]decan-8-yl)-1,5-dihydro-4H-pyrazolo[3,4-d]pyrimidin-4-one

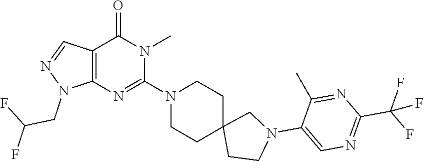 [SMILES: Cc1nc(C(F)(F)F)ncc1N1CCC2(CCN(c3nc4c(cnn4CC(F)F)c(=O)n3C)CC2)C1]

Step 1: tert-butyl 2-(2-(trifluoromethyl)pyrimidin-5-yl)-2,8-diazaspiro[4.5]decane-8-carboxylate: Followed the general procedure Y using tert-butyl 2,8-diazaspiro[4.5]decane-8-carboxylate (600 mg, 2.49 mmol, 1.0 equiv.) and 5-bromo-2-(trifluoromethyl)pyrimidine (566 mg, 2.49 mmol, 1.0 equiv.) as the starting materials, XPhos Pd G3 (105 mg, 0.124 mmol, 0.05 equiv.) as the catalyst to give tert-butyl 2-(2-(trifluoromethyl)pyrimidin-5-yl)-2,8-diazaspiro[4.5]decane-8-carboxylate (870 mg, 80%) as a white solid. MS m/z: 387 [M+H]⁺.

Step 2: tert-butyl 2-(4-chloro-2-(trifluoromethyl)pyrimidin-5-yl)-2,8-diazaspiro[4.5]decane-8-carboxylate: Followed the general procedure V using tert-butyl 2-(2-(trifluoromethyl)pyrimidin-5-yl)-2,8-diazaspiro[4.5]decane-8-carboxylate (870 mg, 2.25 mmol, 1.0 equiv.) as the starting material, NCS (144 mg, 2.47 μmol, 1.1 equiv.) as the reagent to give tert-butyl 2-(4-chloro-2-(trifluoromethyl)pyrimidin-5-yl)-2,8-diazaspiro[4.5]decane-8-carboxylate (710 mg, 60%) as a white solid. MS m/z: 421 [M+H]⁺.

Step 3: 2-(4-methyl-2-(trifluoromethyl)pyrimidin-5-yl)-2,8-diazaspiro[4.5]decane hydrochloride: Followed the general procedure L using tert-butyl 2-(4-chloro-2-(trifluoromethyl)pyrimidin-5-yl)-2,8-diazaspiro[4.5]decane-8-carboxylate (710 mg, 1.69 mmol, 1.0 equiv.) and methylboronic acid (102 mg, 1.69 mol, 1.0 equiv.) as the starting materials, to give tert-butyl 2-(4-methyl-2-(trifluoromethyl)pyrimidin-5-yl)-2,8-diazaspiro[4.5]decane-8-carboxylate (640 mg, 70%) as a white solid. MS m/z: 401 [M+H]⁺.

Step 4: 2-(4-methyl-2-(trifluoromethyl)pyrimidin-5-yl)-2,8-diazaspiro[4.5]decane hydrochloride: Followed the general procedure H using tert-butyl 2-(4-methyl-2-(trifluoromethyl)pyrimidin-5-yl)-2,8-diazaspiro[4.5]decane-8-carboxylate (640 mg) as the starting material to give the crude product 2-(4-methyl-2-(trifluoromethyl)pyrimidin-5-yl)-2,8-diazaspiro[4.5]decane hydrochloride (640 mg). MS m/z: 301[M+H]⁺.

Step 5: 5-methyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure I using 2-(4-methyl-2-(trifluoromethyl)pyrimidin-5-yl)-2,8-diazaspiro[4.5]decane hydrochloride (120 mg, 0.399 mmol, 1.0 equiv.) and 6-chloro-5-methyl-1-(tetrahydro-2H-pyran-2-yl)-1,5-dihydro-4H-pyrazolo[3,4-d]pyrimidin-4-one (107 mg, 0.399 mmol, 1.0 equiv.) as the starting materials to give 5-methyl-6-(2-(4-methyl-2-(trifluoromethyl)pyrimidin-5-yl)-2,8-diazaspiro[4.5]decan-8-yl)-1-(tetrahydro-2H-pyran-2-yl)-1,5-dihydro-4H-pyrazolo[3,4-d]pyrimidin-4-one (180 mg, 50%) as a white solid. MS m/z: 533 [M+H]⁺.

Step 6: 5-methyl-6-(2-(4-methyl-2-(trifluoromethyl)pyrimidin-5-yl)-2,8-diazaspiro[4.5]decan-8-yl)-1,5-dihydro-4H-pyrazolo[3,4-d]pyrimidin-4-one: Followed the general procedure M using 5-methyl-6-(2-(4-methyl-2-(trifluoromethyl)pyrimidin-5-yl)-2,8-diazaspiro[4.5]decan-8-yl)-1-(tetrahydro-2H-pyran-2-yl)-1,5-dihydro-4H-pyrazolo[3,4-d]pyrimidin-4-one (180 mg, 0.338 mmol, 1.0 equiv.) as the starting material to give 5-methyl-6-(2-(4-methyl-2-(trifluoromethyl)pyrimidin-5-yl)-2,8-diazaspiro[4.5]decan-8-yl)-1,5-dihydro-4H-pyrazolo[3,4-d]pyrimidin-4-one (120 mg, 60%) as a white solid. MS m/z: 449 [M+H]⁺.

Step 7: 1-(2,2-difluoroethyl)-5-methyl-6-(2-(4-methyl-2-(trifluoromethyl)pyrimidin-5-yl)-2,8-diazaspiro[4.5]decan-8-yl)-1,5-dihydro-4H-pyrazolo[3,4-d]pyrimidin-4-one: Followed the general procedure K using 5-methyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (110 mg, 0.245 mmol, 1.0 equiv.) and 2,2-difluoroethyl trifluoromethanesulfonate (157 mg, 0.735 mmol, 3.0 equiv.) as the starting materials to give 1-(2,2-difluoroethyl)-5-methyl-6-(2-(4-methyl-2-(trifluoromethyl)pyrimidin-5-yl)-2,8-diazaspiro[4.5]decan-8-yl)-1,5-dihydro-4H-pyrazolo[3,4-d]pyrimidin-4-one (62.8 mg, 60%) as a white solid. ¹H NMR (400 MHz, DMSO-d₆) δ 8.16 (s, 1H), 8.02 (s, 1H), 6.44 (tt, 1H), 4.66 (td, 2H), 3.59 (t, 2H), 3.42 (s, 5H), 3.32-3.22 (m, 4H), 2.65 (s, 3H), 1.91 (t, 2H), 1.75 (q, 4H). MS m/z: 513.20 [M+H]⁺.

### Example 1087: 1-(2,2-difluoroethyl)-5-methyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one

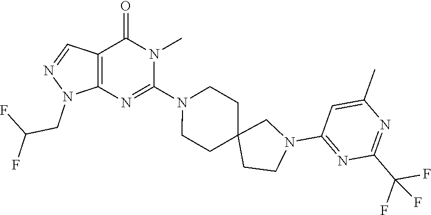 [SMILES: Cc1cc(N2CCC3(CCN(c4nc5c(cnn5CC(F)F)c(=O)n4C)CC3)C2)nc(C(F)(F)F)n1]

Step 1: tert-butyl 2-(6-methyl-2-(trifluoromethyl)pyrimidin-4-yl)-2,8-diazaspiro[4.5]decane-8-carboxylate: Followed the general procedure I using tert-butyl 2,8-diazaspiro[4.5]decane-8-carboxylate (250 mg, 1.04 mmol, 1.0 equiv.) and 4-chloro-6-methyl-2-(trifluoromethyl)pyrimidine (245.4 mg, 1.25 mmol, 1.2 equiv.) as the starting materials to give tert-butyl 2-(6-methyl-2-(trifluoromethyl)pyrimidin-4-yl)-2,8-diazaspiro[4.5]decane-8-carboxylate (360 mg, 86%) as a white solid. MS m/z: 401 [M+H]⁺.

Step 2: 2-(6-methyl-2-(trifluoromethyl)pyrimidin-4-yl)-2,8-diazaspiro[4.5]decane hydrochloride: Followed the general procedure B using tert-butyl 2-(6-methyl-2-(trifluoromethyl)pyrimidin-4-yl)-2,8-diazaspiro[4.5]decane-8-carboxylate (350 mg) as the starting material to give the crude product 2-(6-methyl-2-(trifluoromethyl)pyrimidin-4-yl)-2,8-diazaspiro[4.5]decane hydrochloride (295 mg). MS m/z: 301 [M+H]⁺.

Step 3: 5-methyl-6-(2-(6-methyl-2-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one: Followed the general procedure I using 2-(6-methyl-2-(trifluoromethyl)pyrimidin-4-yl)-2,8-diazaspiro[4.5]decane hydrochloride (110 mg, 366 μmol, 1.0 equiv.) and 6-chloro-5-methyl-1-(tetrahydro-2H-pyran-2-yl)-1,5-dihydro-4H-pyrazolo[3,4-d]pyrimidin-4-one (98.4 mg, 366 μmol, 1.0 equiv.) as the starting materials to give 5-methyl-6-(2-(6-methyl-2-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (190 mg, 97%) as a white solid. MS m/z: 533 [M+H]⁺.

Step 4: 5-methyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure M using 5-methyl-6-(2-(6-methyl-2-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (170 mg, 319 μmol, 1.0 equiv.) as the starting material to give 5-methyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one (70 mg, 19%) as a white solid. MS m/z: 449 [M+H]⁺.

Step 5: 1-(2,2-difluoroethyl)-5-methyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure K using 5-methyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one (70 mg, 156 μmol, 1.0 equiv.) and 2,2-difluoroethyl trifluoromethanesulfonate (66.8 mg, 312 μmol, 2.0 equiv.) as the starting materials to give 1-(2,2-difluoroethyl)-5-methyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one (26.8 mg, 33%) as a white solid. ¹H NMR (400 MHz, DMSO-d₆) δ 8.03 (d, J=2.0 Hz, 1H), 6.70-6.23 (m, 2H), 4.63 (td, J=14.9, 3.8 Hz, 2H), 3.61 (d, J=7.5 Hz, 1H), 3.48 (d, J=10.7 Hz, 2H), 3.43 (s, 3H), 3.38 (s, 1H), 3.22 (s, 3H), 2.38-2.30 (m, 3H), 2.04-1.86 (m, 2H), 1.74 (s, 4H). MS m/z: 513.1 [M+H]⁺.

### Example 1088: 1-(2,2-difluoroethyl)-6-(2-(2-(1,1-difluoroethyl)pyrimidin-5-yl)-2,6-diazaspiro[3.4]octan-6-yl)-1H-pyrazolo[3,4-b]pyrazine

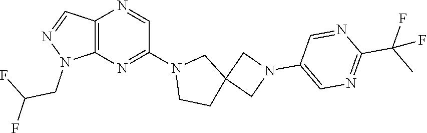 [SMILES: CC(F)(F)c1ncc(N2CC3(CCN(c4cnc5cnn(CC(F)F)c5n4)C3)C2)cn1]

Step 1: 5-bromo-2-(1,1-difluoroethyl)pyrimidine: Followed the general procedure AD using 1-(5-bromopyrimidin-2-yl)ethan-1-one (200 mg, 0.995 mmol, 1.0 equiv.) as the starting material to give 5-bromo-2-(1,1-difluoroethyl)pyrimidine (120 mg, 54%) as a white solid. MS m/z: 223 [M+H]⁺.

Step 2: tert-butyl 2-(2-(1,1-difluoroethyl)pyrimidin-5-yl)-2,6-diazaspiro[3.4]octane-6-carboxylate: Followed the general procedure Y using 5-bromo-2-(1,1-difluoroethyl)pyrimidine (120 mg, 0.538 mmol, 1.0 equiv.) and tert-butyl 2,6-diazaspiro[3.4]octane-6-carboxylate (115 mg, 0.538 mmol, 1.0 equiv.) as the starting materials to give tert-butyl 2-(2-(1,1-difluoroethyl)pyrimidin-5-yl)-2,6-diazaspiro[3.4]octane-6-carboxylate (153 mg, 80%) as a white solid. MS m/z: 355 [M+H]⁺.

Step 3: 2-(2-(1,1-difluoroethyl)pyrimidin-5-yl)-2,6-diazaspiro[3.4]octane: Followed the general procedure H using tert-butyl 2-(2-(1,1-difluoroethyl)pyrimidin-5-yl)-2,6-diazaspiro[3.4]octane-6-carboxylate (153 mg) as the starting material to give the crude product 2-(2-(1,1-difluoroethyl)pyrimidin-5-yl)-2,6-diazaspiro[3.4]octane (153 mg). MS m/z: 255 [M+H]⁺.

Step 4: 1-(2,2-difluoroethyl)-6-(2-(2-(1,1-difluoroethyl)pyrimidin-5-yl)-2,6-diazaspiro[3.4]octan-6-yl)-1H-pyrazolo[3,4-b]pyrazine: Followed the general procedure I using 2-(2-(1,1-difluoroethyl)pyrimidin-5-yl)-2,6-diazaspiro[3.4]octane (110 mg, 0.431 mmol, 1.0 equiv.) and 6-chloro-1-(2,2-difluoroethyl)-1H-pyrazolo[3,4-b]pyrazine (94.5 mg, 0.431 mmol, 1.0 equiv.) as the starting materials to give 1-(2,2-difluoroethyl)-6-(2-(2-(1,1-difluoroethyl)pyrimidin-5-yl)-2,6-diazaspiro[3.4]octan-6-yl)-1H-pyrazolo[3,4-b]pyrazine (112.8 mg, 59%) as a white solid. ¹H NMR (300 MHz, DMSO-d₆) δ 8.20-8.05 (d, 4H), 6.46 (tt, 1H), 4.68 (td, 2H), 4.15-3.96 (m, 4H), 3.86 (s, 2H), 3.67 (t, 2H), 2.33 (t, 2H), 1.99 (t, 3H). MS m/z: 437.3 [M+H]⁺.

### Example 1089: 1-(2,2-difluoroethyl)-6-(2-(2-ethyl-6-(trifluoromethyl)pyrimidin-4-yl)-2,6-diazaspiro[3.4]octan-6-yl)-1H-pyrazolo[3,4-b]pyrazine

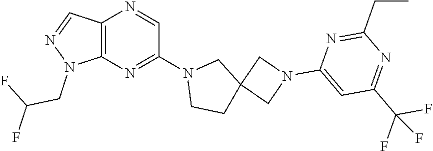 [SMILES: CCc1nc(N2CC3(CCN(c4cnc5cnn(CC(F)F)c5n4)C3)C2)cc(C(F)(F)F)n1]

Step 1: tert-butyl 2-(2-chloro-6-(trifluoromethyl)pyrimidin-4-yl)-2,6-diazaspiro[3.4]octane-6-carboxylate: Followed the general procedure I using tert-butyl 2,6-diazaspiro[3.4]octane-6-carboxylate (320 mg, 1.5 mmol, 1.0 equiv.) and 2,4-dichloro-6-(trifluoromethyl)pyrimidine (391 mg, 1.8 mmol, 1.2 equiv.) as the starting materials to give tert-butyl 2-(2-chloro-6-(trifluoromethyl)pyrimidin-4-yl)-2,6-diazaspiro[3.4]octane-6-carboxylate (380 mg, 65%) as a colorless oil. MS m/z: 393[M+H]⁺.

Step 2: tert-butyl 2-(6-(trifluoromethyl)-2-vinylpyrimidin-4-yl)-2,6-diazaspiro[3.4]octane-6-carboxylate: Followed the general procedure L using tert-butyl 2-(2-chloro-6-(trifluoromethyl)pyrimidin-4-yl)-2,6-diazaspiro[3.4]octane-6-carboxylate (380 mg, 0.97 mmol, 1.0 equiv.) and 4,4,5,5-tetramethyl-2-vinyl-1,3,2-dioxaborolane (179 mg, 1.16 mmol, 1.2 equiv.) as the starting materials to give tert-butyl 2-(6-(trifluoromethyl)-2-vinylpyrimidin-4-yl)-2,6-diazaspiro[3.4]octane-6-carboxylate (250 mg, 67%) as a white solid. MS m/z: 385[M+H]⁺.

Step 3: tert-butyl 2-(2-ethyl-6-(trifluoromethyl)pyrimidin-4-yl)-2,6-diazaspiro[3.4]octane-6-carboxylate: Followed the general procedure U using tert-butyl 2-(6-(trifluoromethyl)-2-vinylpyrimidin-4-yl)-2,6-diazaspiro[3.4]octane-6-carboxylate (250 mg, 0.65 mmol, 1.0 equiv.) as the starting material to give tert-butyl 2-(2-ethyl-6-(trifluoromethyl)pyrimidin-4-yl)-2,6-diazaspiro[3.4]octane-6-carboxylate (200 mg, 80%) as a white solid. MS m/z: 387 [M+H]⁺.

Step 4: 2-(2-ethyl-6-(trifluoromethyl)pyrimidin-4-yl)-2,6-diazaspiro[3.4]octane: Followed the general procedure H using tert-butyl 2-(2-ethyl-6-(trifluoromethyl)pyrimidin-4-yl)-2,6-diazaspiro[3.4]octane-6-carboxylate (200 mg) as the starting material to give the crude product 2-(2-ethyl-6-(trifluoromethyl)pyrimidin-4-yl)-2,6-diazaspiro[3.4]octane (80 mg). MS m/z: 287 [M+H]⁺.

Step 5: 1-(2,2-difluoroethyl)-6-(2-(2-ethyl-6-(trifluoromethyl)pyrimidin-4-yl)-2,6-diazaspiro[3.4]octan-6-yl)-1H-pyrazolo[3,4-b]pyrazine: Followed the general procedure I using 2-(2-ethyl-6-(trifluoromethyl)pyrimidin-4-yl)-2,6-diazaspiro[3.4]octane (80 mg, 0.279 mmol, 1.0 equiv.) and 6-chloro-1-(2,2-difluoroethyl)-1H-pyrazolo[3,4-b]pyrazine (51 mg, 0.232 mmol, 1.2 equiv.) as the starting materials to give 1-(2,2-difluoroethyl)-6-(2-(2-ethyl-6-(trifluoromethyl)pyrimidin-4-yl)-2,6-diazaspiro[3.4]octan-6-yl)-1H-pyrazolo[3,4-b]pyrazine (52.9 mg, 40%) as a white solid. ¹H NMR (400 MHz, DMSO-d₆) δ 8.16-8.07 (m, 2H), 6.67 (s, 1H), 6.62-6.30 (m, 1H), 4.75-4.62 (m, 2H), 4.23-4.10 (m, 4H), 3.87 (s, 2H), 3.68 (t, J=6.8 Hz, 2H), 2.70 (q, J=7.6 Hz, 2H), 2.32 (t, J=6.8 Hz, 2H), 1.22 (t, J=7.6 Hz, 3H). MS m/z: 469.1 [M+H]⁺.

### Example 1090: 1-(tetrahydro-2H-pyran-4-yl)-6-(2-(2-(trifluoromethyl)pyridin-4-yl)-2,6-diazaspiro[3.4]octan-6-yl)-1H-pyrazolo[3,4-b]pyrazine

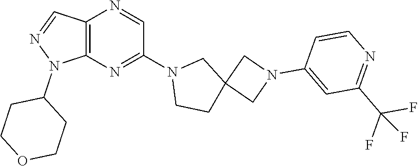 [SMILES: FC(F)(F)c1cc(N2CC3(CCN(c4cnc5cnn(C6CCOCC6)c5n4)C3)C2)ccn1]

Step 1: 6-chloro-1-(tetrahydro-2H-pyran-4-yl)-1H-pyrazolo[3,4-b]pyrazine: Followed the general procedure K using 6-chloro-1H-pyrazolo[3,4-b]pyrazine (100 mg, 0.648 mmol, 1.0 equiv.) and 4-iodotetrahydro-2H-pyran (165 mg, 0.778 mmol, 1.2 equiv.) as the starting materials to give 6-chloro-1-(tetrahydro-2H-pyran-4-yl)-1H-pyrazolo[3,4-b]pyrazine (45 mg, 29%) as a light yellow solid. MS m/z: 239 [M+H]⁺

Step 2: 1-(tetrahydro-2H-pyran-4-yl)-6-(2-(2-(trifluoromethyl)pyridin-4-yl)-2,6-diazaspiro[3.4]octan-6-yl)-1H-pyrazolo[3,4-b]pyrazine: Followed the general procedure I using 6-chloro-1-(tetrahydro-2H-pyran-4-yl)-1H-pyrazolo[3,4-b]pyrazine (40 mg, 0.168 mmol, 1.0 equiv.) and 2-(2-(trifluoromethyl)pyridin-4-yl)-2,6-diazaspiro[3.4]octane hydrochloride (49.2 mg, 0.168 mmol, 1.0 equiv.) as the starting materials to give 1-(tetrahydro-2H-pyran-4-yl)-6-(2-(2-(trifluoromethyl)pyridin-4-yl)-2,6-diazaspiro[3.4]octan-6-yl)-1H-pyrazolo[3,4-b]pyrazine (19.3 mg, 24%) as a white solid. ¹H NMR (400 MHz, DMSO-d₆) δ 8.25 (d, J=5.6 Hz, 1H), 8.06 (d, J=6.5 Hz, 1H), 6.76 (d, J=2.3 Hz, 1H), 6.59-6.57 (m, 1H), 4.79-4.71 (m, 1H), 4.09-3.97 (m, 6H), 3.85 (s, 2H), 3.66 (t, J=6.9 Hz, 2H), 3.55-3.49 (m, 2H), 2.31 (t, J=6.8 Hz, 2H), 2.25-2.15 (m, 2H), 1.87-1.83 (m, 2H), 1.24-1.15 (m, 1H). MS m/z: 460.15 [M+H]⁺.

### Example 1091: (R)-1-(tetrahydrofuran-3-yl)-6-(2-(2-(trifluoromethyl)pyridin-4-yl)-2,6-diazaspiro[3.4]octan-6-yl)-1H-pyrazolo[3,4-b]pyrazine

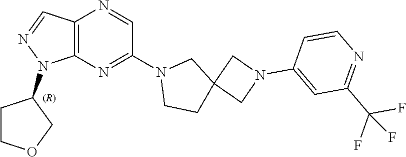

Step 1: (R)-6-chloro-1-(tetrahydrofuran-3-yl)-1H-pyrazolo[3,4-b]pyrazine: Followed the general procedure C using 6-chloro-1H-pyrazolo[3,4-b]pyrazine (200 mg, 1.29 mmol, 1.0 equiv.) and (S)-tetrahydrofuran-3-ol (137 mg, 1.55 mmol, 1.2 equiv.) as the starting materials to give (R)-6-chloro-1-(tetrahydrofuran-3-yl)-1H-pyrazolo[3,4-b]pyrazine (230 mg, 79%) as a yellow solid. MS m/z: 224 [M+H]⁺.

Step 2: (R)-1-(tetrahydrofuran-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure I using (R)-6-chloro-1-(tetrahydrofuran-3-yl)-1H-pyrazolo[3,4-b]pyrazine (80 mg, 0.356 mmol, 1.0 equiv.) and 2-(2-(trifluoromethyl)pyridin-4-yl)-2,6-diazaspiro[3.4]octane (101 mg, 0.392 mmol, 1.1 equiv.) as the starting materials to give (R)-1-(tetrahydrofuran-3-yl)-6-(2-(2-(trifluoromethyl)pyridin-4-yl)-2,6-diazaspiro[3.4]octan-6-yl)-1H-pyrazolo[3,4-b]pyrazine (44 mg, 27%) as a white solid. ¹H NMR (400 MHz, DMSO-d₆) δ 8.25 (d, J=5.7 Hz, 1H), 8.07 (d, J=3.1 Hz, 2H), 6.76 (d, J=2.3 Hz, 1H), 6.58 (s, 1H), 5.36-5.21 (m, 1H), 4.11-3.99 (m, 6H), 3.93-3.81 (m, 4H), 3.66 (t, J=6.9 Hz, 2H), 2.42-2.35 (m, 2H), 2.31 (t, J=6.8 Hz, 2H). MS m/z: 446.15 [M+H]⁺.

### Example 1092: (S)-1-(tetrahydrofuran-3-yl)-6-(2-(2-(trifluoromethyl)pyridin-4-yl)-2,6-diazaspiro[3.4]octan-6-yl)-1H-pyrazolo[3,4-b]pyrazine

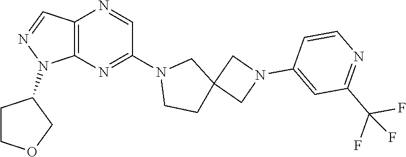 [SMILES: FC(F)(F)c1cc(N2CC3(CCN(c4cnc5cnn([C@H]6CCOC6)c5n4)C3)C2)ccn1]

Step 1: (S)-6-chloro-1-(tetrahydrofuran-3-yl)-1H-pyrazolo[3,4-b]pyrazine: Followed the general procedure C using 6-chloro-1H-pyrazolo[3,4-b]pyrazine (220 mg, 1.42 mmol, 1.0 equiv.) and (R)-tetrahydrofuran-3-ol (150 mg, 1.70 mmol, 1.2 equiv.) as the starting materials to give (S)-6-chloro-1-(tetrahydrofuran-3-yl)-1H-pyrazolo[3,4-b]pyrazine (230 mg, 71%) as a light yellow solid. MS m/z: 225 [M+H]⁺

Step 2: (S)-1-(tetrahydrofuran-3-yl)-6-(2-(2-(trifluoromethyl)pyridin-4-yl)-2,6-diazaspiro[3.4]octan-6-yl)-1H-pyrazolo[3,4-b]pyrazine: Followed the general procedure I using 2-(2-(trifluoromethyl)pyridin-4-yl)-2,6-diazaspiro[3.4]octane hydrochloride (120 mg, 0.409 mmol, 1.0 equiv.) and (S)-6-chloro-1-(tetrahydrofuran-3-yl)-1H-pyrazolo[3,4-b]pyrazine (110 mg, 0.490 mmol, 1.2 equiv.) as the starting materials to give (S)-1-(tetrahydrofuran-3-yl)-6-(2-(2-(trifluoromethyl)pyridin-4-yl)-2,6-diazaspiro[3.4]octan-6-yl)-1H-pyrazolo[3,4-b]pyrazine (80 mg, 43%) as a white solid. ¹H NMR (300 MHz, DMSO-d₆) δ 8.26 (d, J=5.7 Hz, 1H), 8.08 (d, J=2.4 Hz, 2H), 6.77 (d, J=2.2 Hz, 1H), 6.58 (d, J=5.4 Hz, 1H), 5.36-5.31 (m, 1H), 4.12-4.01 (m, 6H), 3.94-3.86 (m, 4H), 3.67 (t, J=6.9 Hz, 2H), 2.40 (t, J=7.0 Hz, 2H), 2.34-2.28 (m, 2H). MS m/z: 446.2 [M+H]⁺.

### Example 1093: 1-(2,2-difluoroethyl)-3-methoxy-6-(2-(4-methyl-2-(trifluoromethyl)pyrimidin-5-yl)-2,6-diazaspiro[3.4]octan-6-yl)-1H-pyrazolo[3,4-b]pyrazine

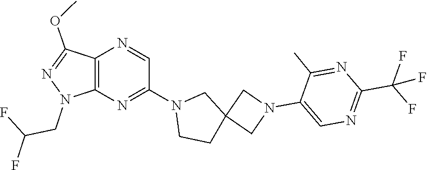 [SMILES: COc1nn(CC(F)F)c2nc(N3CCC4(C3)CN(c3cnc(C(F)(F)F)nc3C)C4)cnc12]

Step 1: 3-iodo-6-(2-(4-methyl-2-(trifluoromethyl)pyrimidin-5-yl)-2,6-diazaspiro[3.4]octan-6-yl)-1H-pyrazolo[3,4-b]pyrazine: Followed the general procedure I using 2-(4-methyl-2-(trifluoromethyl)pyrimidin-5-yl)-2,6-diazaspiro[3.4]octane (80.0 mg, 0.293 mmol, 1.0 equiv.) and 6-chloro-3-iodo-1H-pyrazolo[3,4-b]pyrazine (82.3 mg, 0.293 mmol, 1.0 equiv.) as the starting materials to give 3-iodo-6-(2-(4-methyl-2-(trifluoromethyl)pyrimidin-5-yl)-2,6-diazaspiro[3.4]octan-6-yl)-1H-pyrazolo[3,4-b]pyrazine (130 mg, 86%) as a white solid. MS m/z: 517 [M+H]⁺.

Step 2: 1-(2,2-difluoroethyl)-3-iodo-6-(2-(4-methyl-2-(trifluoromethyl)pyrimidin-5-yl)-2,6-diazaspiro[3.4]octan-6-yl)-1H-pyrazolo[3,4-b]pyrazine: Followed the general procedure K using 3-iodo-6-(2-(4-methyl-2-(trifluoromethyl)pyrimidin-5-yl)-2,6-diazaspiro[3.4]octan-6-yl)-1H-pyrazolo[3,4-b]pyrazine (120 mg, 0.232 mmol, 1.0 equiv.) and 2,2-difluoroethyl trifluoromethanesulfonate (149 mg, 0.696 mmol, 3.0 equiv.) as the starting materials to give 1-(2,2-difluoroethyl)-3-iodo-6-(2-(4-methyl-2-(trifluoromethyl)pyrimidin-5-yl)-2,6-diazaspiro[3.4]octan-6-yl)-1H-pyrazolo[3,4-b]pyrazine (90 mg, 65%) as a white solid. MS m/z: 581 [M+H]⁺.

Step 3: 1-(2,2-difluoroethyl)-3-methoxy-6-(2-(4-methyl-2-(trifluoromethyl)pyrimidin-5-yl)-2,6-diazaspiro[3.4]octan-6-yl)-1H-pyrazolo[3,4-b]pyrazine: Followed the general procedure J using 1-(2,2-difluoroethyl)-3-iodo-6-(2-(4-methyl-2-(trifluoromethyl)pyrimidin-5-yl)-2,6-diazaspiro[3.4]octan-6-yl)-1H-pyrazolo[3,4-b]pyrazine (80.0 mg, 0.138 mmol, 1.0 equiv.) and MeOH (4.41 mg, 0.138 mmol, 1.0 equiv.) as the starting materials to give 1-(2,2-difluoroethyl)-3-methoxy-6-(2-(4-methyl-2-(trifluoromethyl)pyrimidin-5-yl)-2,6-diazaspiro[3.4]octan-6-yl)-1H-pyrazolo[3,4-b]pyrazine (55.8 mg, 85%) as a white solid. ¹H NMR (400 MHz, DMSO-d₆) δ 7.97 (d, 2H), 6.38 (tt, 1H), 4.49 (td, 2H), 4.27-4.02 (m, 4H), 3.99 (s, 3H), 3.84 (s, 2H), 3.66 (t, 2H), 2.46 (s, 3H), 2.32 (d, 2H). MS m/z: 485.1 [M+H]⁺.

### Example 1094: 8-(1-(2,2-difluoroethyl)-3-methoxy-1H-pyrazolo[3,4-b]pyrazin-6-yl)-2-(4-methyl-2-(trifluoromethyl)pyrimidin-5-yl)-2,8-diazaspiro[4.5]decane

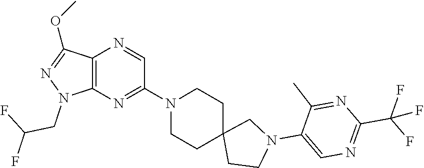 [SMILES: COc1nn(CC(F)F)c2nc(N3CCC4(CC3)CCN(c3cnc(C(F)(F)F)nc3C)C4)cnc12]

Step 1: 8-(3-iodo-1-(tetrahydro-2H-pyran-2-yl)-1H-pyrazolo[3,4-b]pyrazin-6-yl)-2-(4-methyl-2-(trifluoromethyl)pyrimidin-5-yl)-2,8-diazaspiro[4.5]decane: Followed the general procedure I using 2-(4-methyl-2-(trifluoromethyl)pyrimidin-5-yl)-2,8-diazaspiro[4.5]decane (120 mg, 0.399 mmol, 1.0 equiv.) and 6-chloro-3-iodo-1-(tetrahydro-2H-pyran-2-yl)-1H-pyrazolo[3,4-b]pyrazine (146 mg, 0.399 mmol, 1.0 equiv.) as the starting materials to give 8-(3-iodo-1-(tetrahydro-2H-pyran-2-yl)-1H-pyrazolo[3,4-b]pyrazin-6-yl)-2-(4-methyl-2-(trifluoromethyl)pyrimidin-5-yl)-2,8-diazaspiro[4.5]decane (220 mg, 88%) as a white solid. MS m/z: 628 [M+H]⁺.

Step 2: 8-(3-methoxy-1-(tetrahydro-2H-pyran-2-yl)-1H-pyrazolo[3,4-b]pyrazin-6-yl)-2-(4-methyl-2-(trifluoromethyl)pyrimidin-5-yl)-2,8-diazaspiro[4.5]decane: Followed the general procedure J using 8-(3-iodo-1-(tetrahydro-2H-pyran-2-yl)-1H-pyrazolo[3,4-b]pyrazin-6-yl)-2-(4-methyl-2-(trifluoromethyl)pyrimidin-5-yl)-2,8-diazaspiro[4.5]decane (210 mg, 0.334 mmol, 1.0 equiv.) and MeOH (11 mg, 0.334 mmol, 1.0 equiv.) as the starting materials to give 8-(3-methoxy-1-(tetrahydro-2H-pyran-2-yl)-1H-pyrazolo[3,4-b]pyrazin-6-yl)-2-(4-methyl-2-(trifluoromethyl)pyrimidin-5-yl)-2,8-diazaspiro[4.5]decane (140 mg, 79%) as a white solid. MS m/z: 533 [M+H⁺.

Step 3: 8-(3-methoxy-1H-pyrazolo[3,4-b]pyrazin-6-yl)-2-(4-methyl-2-(trifluoromethyl)pyrimidin-5-yl)-2,8-diazaspiro[4.5]decane: Followed the general procedure M using 8-(3-methoxy-1-(tetrahydro-2H-pyran-2-yl)-1H-pyrazolo[3,4-b]pyrazin-6-yl)-2-(4-methyl-2-(trifluoromethyl)pyrimidin-5-yl)-2,8-diazaspiro[4.5]decane (130 mg, 0.244 mmol, 1.0 equiv.) as the starting material to give 8-(3-methoxy-1H-pyrazolo[3,4-b]pyrazin-6-yl)-2-(4-methyl-2-(trifluoromethyl)pyrimidin-5-yl)-2,8-diazaspiro[4.5]decane (90 mg, 82%) as a white solid. MS m/z: 449 [M+H]⁺.

Step 4: 8-(1-(2,2-difluoroethyl)-3-methoxy-1H-pyrazolo[3,4-b]pyrazin-6-yl)-2-(4-methyl-2-(trifluoromethyl)pyrimidin-5-yl)-2,8-diazaspiro[4.5]decane: Followed the general procedure K using 8-(3-methoxy-1H-pyrazolo[3,4-b]pyrazin-6-yl)-2-(4-methyl-2-(trifluoromethyl)pyrimidin-5-yl)-2,8-diazaspiro[4.5]decane (80 mg, 0.178 mmol, 1.0 equiv.) and 2,2-difluoroethyl trifluoromethanesulfonate (115 mg, 0.535 mol, 3.0 equiv.) as the starting materials to give 8-(1-(2,2-difluoroethyl)-3-methoxy-1H-pyrazolo[3,4-b]pyrazin-6-yl)-2-(4-methyl-2-(trifluoromethyl)pyrimidin-5-yl)-2,8-diazaspiro[4.5]decane (23.6 mg, 26%) as a white solid. ¹H NMR (400 MHz, DMSO-d₆) δ 8.34 (s, 1H), 8.14 (s, 1H), 6.37 (tt, 1H), 4.52 (td, 2H), 3.99 (s, 3H), 3.90-3.68 (m, 4H), 3.60 (t, 2H), 3.42 (s, 2H), 2.64 (s, 3H), 1.90 (t, 2H), 1.67 (p, 4H). MS m/z: 513.2 [M+H]⁺.

### Example 1095: 1-(2,2-difluoroethyl)-3-methoxy-6-(2-(2-methyl-6-(trifluoromethyl)pyrimidin-4-yl)-2,6-diazaspiro[3.4]octan-6-yl)-1H-pyrazolo[3,4-b]pyrazine

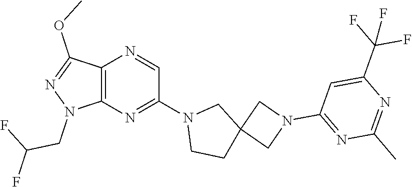 [SMILES: COc1nn(CC(F)F)c2nc(N3CCC4(CN(c5cc(C(F)(F)F)nc(C)n5)C4)C3)cnc12]

Step 1: 3-iodo-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure I using 2-[2-methyl-6-(trifluoromethyl)pyrimidin-4-yl]-2,6-diazaspiro[3.4]octane (200 mg, 0.735 mmol, 1.0 equiv.) and 6-chloro-3-iodo-1H-pyrazolo[3,4-b]pyrazine (247 mg, 0.882 mmol, 1.2 equiv.) as the starting materials to give 3-iodo-6-(2-(2-methyl-6-(trifluoromethyl)pyrimidin-4-yl)-2,6-diazaspiro[3.4]octan-6-yl)-1H-pyrazolo[3,4-b]pyrazine (100 mg, 26%) as a white solid. MS m/z: 517 [M+H]⁺.

Step 2: 1-(2,2-difluoroethyl)-3-iodo-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure K using 3-iodo-6-(2-(2-methyl-6-(trifluoromethyl)pyrimidin-4-yl)-2,6-diazaspiro[3.4]octan-6-yl)-1H-pyrazolo[3,4-b]pyrazine (100 mg, 0.194 mmol, 1.0 equiv.) and 2,2-difluoroethyl trifluoromethanesulfonate (49.7 mg, 0.233 mmol, 1.2 equiv.) as the starting materials to give 1-(2,2-difluoroethyl)-3-iodo-6-(2-(2-methyl-6-(trifluoromethyl)pyrimidin-4-yl)-2,6-diazaspiro[3.4]octan-6-yl)-1H-pyrazolo[3,4-b]pyrazine (100 mg, 36.9%) as a white solid. MS m/z: 581 [M+H]⁺.

Step 3: 1-(2,2-difluoroethyl)-5-ethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure J using 6-[1-(2,2-difluoroethyl)-3-iodopyrazolo[3,4-b]pyrazin-6-yl]-2-[2-methyl-6-(trifluoromethyl)pyrimidin-4-yl]-2,6-diazaspiro[3.4]octane (100 mg, 172 μmol, 1.0 equiv.) and MeOH (11 mg, 344 μmol, 2.0 equiv.) as the starting materials to give 1-(2,2-difluoroethyl)-3-methoxy-6-(2-(2-methyl-6-(trifluoromethyl)pyrimidin-4-yl)-2,6-diazaspiro[3.4]octan-6-yl)-1H-pyrazolo[3,4-b]pyrazine (57.2 mg, 56.2%) as a white solid. ¹H NMR (400 MHz, DMSO-d₆) δ 7.95 (s, 1H), 6.66 (s, 1H), 6.38 (tt, J=55.1, 3.9 Hz, 1H), 4.50 (td, J=14.7, 4.0 Hz, 2H), 4.20-4.08 (m, 4H), 3.99 (s, 3H), 3.84 (s, 2H), 3.65 (t, J=6.9 Hz, 2H), 2.43 (s, 3H), 2.29 (s, 2H). MS m/z: 485.10 [M+H]⁺.

### Example 1096: 8-(1-(2,2-difluoroethyl)-3-methoxy-1H-pyrazolo[3,4-b]pyrazin-6-yl)-2-(2-methyl-6-(trifluoromethyl)pyrimidin-4-yl)-2,8-diazaspiro[4.5]decane

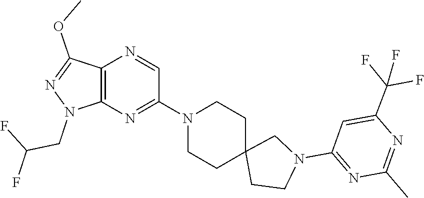 [SMILES: COc1nn(CC(F)F)c2nc(N3CCC4(CCN(c5cc(C(F)(F)F)nc(C)n5)C4)CC3)cnc12]

Step 1: 8-(3-iodo-1-(tetrahydro-2H-pyran-2-yl)-1H-pyrazolo[3,4-b]pyrazin-6-yl)-2-(2-methyl-6-(trifluoromethyl)pyrimidin-4-yl)-2,8-diazaspiro[4.5]decane: Followed the general procedure I using 2-(2-methyl-6-(trifluoromethyl)pyrimidin-4-yl)-2,8-diazaspiro[4.5]decane (197 mg, 0.659 mmol, 1.2 equiv.) and 6-chloro-3-iodo-1-(tetrahydro-2H-pyran-2-yl)-1H-pyrazolo[3,4-b]pyrazine (200 mg, 0.549 mmol, 1.0 equiv.) as the starting materials to give 8-(3-iodo-1-(tetrahydro-2H-pyran-2-yl)-1H-pyrazolo[3,4-b]pyrazin-6-yl)-2-(2-methyl-6-(trifluoromethyl)pyrimidin-4-yl)-2,8-diazaspiro[4.5]decane (220 mg, 56%) as a light yellow oil.. MS m/z: 629 [M+H]⁺

Step 2: 8-(3-methoxy-1-(tetrahydro-2H-pyran-2-yl)-1H-pyrazolo[3,4-b]pyrazin-6-yl)-2-(2-methyl-6-(trifluoromethyl)pyrimidin-4-yl)-2,8-diazaspiro[4.5]decane: Followed the general procedure J using 8-(3-iodo-1-(tetrahydro-2H-pyran-2-yl)-1H-pyrazolo[3,4-b]pyrazin-6-yl)-2-(2-methyl-6-(trifluoromethyl)pyrimidin-4-yl)-2,8-diazaspiro[4.5]decane (220 mg, 0.350 mmol, 1.0 equiv.) and MeOH (22 mg, 0.700 mmol, 2.0 equiv.) as the starting materials to give 8-(3-methoxy-1-(tetrahydro-2H-pyran-2-yl)-1H-pyrazolo[3,4-b]pyrazin-6-yl)-2-(2-methyl-6-(trifluoromethyl)pyrimidin-4-yl)-2,8-diazaspiro[4.5]decane (200 mg, 34%) as colorless oil. MS m/z: 533 [M+H]⁺.

Step 3: 8-(3-methoxyl-1H-pyrazolo[3,4-b]pyrazin-6-yl)-2-(2-methyl-6-(trifluoromethyl)pyrimidin-4-yl)-2,8-diazaspiro[4.5]decane: Followed the general procedure M using 8-(3-methoxy-1-(tetrahydro-2H-pyran-2-yl)-1H-pyrazolo[3,4-b]pyrazin-6-yl)-2-(2-methyl-6-(trifluoromethyl)pyrimidin-4-yl)-2,8-diazaspiro[4.5]decane (180 mg, 0.485 mmol, 1.0 equiv.) as the starting material to give 8-(3-methoxy-1H-pyrazolo[3,4-b]pyrazin-6-yl)-2-(2-methyl-6-(trifluoromethyl)pyrimidin-4-yl)-2,8-diazaspiro[4.5]decane (100 mg, 92%) as a light yellow oil. MS m/z: 449 [M+H]⁺.

Step 4: 8-(1-(2,2-difluoroethyl)-3-methoxy-1H-pyrazolo[3,4-b]pyrazin-6-yl)-2-(2-methyl-6-(trifluoromethyl)pyrimidin-4-yl)-2,8-diazaspiro[4.5]decane: Followed the general procedure K using 8-(3-methoxy-1H-pyrazolo[3,4-b]pyrazin-6-yl)-2-(2-methyl-6-(trifluoromethyl)pyrimidin-4-yl)-2,8-diazaspiro[4.5]decane (100 mg, 0.446 mmol, 1.0 equiv.) and 2,2-difluoroethyl trifluoromethanesulfonate (250 mg, 2.23 mmol, 5.0 equiv.) as the starting materials to give 8-(1-(2,2-difluoroethyl)-3-methoxy-1H-pyrazolo[3,4-b]pyrazin-6-yl)-2-(2-methyl-6-(trifluoromethyl)pyrimidin-4-yl)-2,8-diazaspiro[4.5]decane (25.4 mg, 11%) as a white solid. ¹H NMR (400 MHz, DMSO-d₆) δ 8.34 (d, J=2.7 Hz, 1H), 6.74 (d, J=20.7 Hz, 1H), 6.60-6.20 (m, 1H), 4.57-4.45 (m, 2H), 3.99 (s, 3H), 3.93-3.89 (m, 1H), 3.79 (t, J=5.9 Hz, 2H), 3.65 (t, J=7.3 Hz, 2H), 3.53 (d, J=14.7 Hz, 2H), 3.44 (s, 1H), 2.43 (d, J=2.3 Hz, 3H), 1.98 (t, J=7.0 Hz, 1H), 1.89 (t, J=7.1 Hz, 1H), 1.75-1.50 (m, 4H). MS m/z: 513.05 [M+H]⁺.

### Example 1097: 1-(2,2-difluoroethyl)-3-methoxy-6-(2-(6-methyl-2-(trifluoromethyl)pyrimidin-4-yl)-2,6-diazaspiro[3.4]octan-6-yl)-1H-pyrazolo[3,4-b]pyrazine

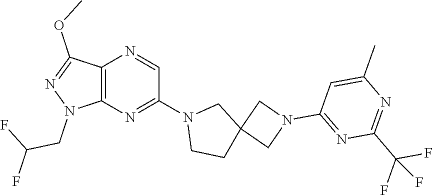 [SMILES: COc1nn(CC(F)F)c2nc(N3CCC4(CN(c5cc(C)nc(C(F)(F)F)n5)C4)C3)cnc12]

Step 1: 1-(2,2-difluoroethyl)-3-iodo-6-(2-(6-methyl-2-(trifluoromethyl)pyrimidin-4-yl)-2,6-diazaspiro[3.4]octan-6-yl)-1H-pyrazolo[3,4-b]pyrazine: Followed the general procedure I using 2-(6-methyl-2-(trifluoromethyl)pyrimidin-4-yl)-2,6-diazaspiro[3.4]octane (100 mg, 0.37 mmol, 1.0 equiv.) and 6-chloro-1-(2,2-difluoroethyl)-3-iodo-1H-pyrazolo[3,4-b]pyrazine (139 mg, 0.41 mmol, 1.1 equiv.) as the starting materials to give 1-(2,2-difluoroethyl)-3-iodo-6-(2-(6-methyl-2-(trifluoromethyl)pyrimidin-4-yl)-2,6-diazaspiro[3.4]octan-6-yl)-1H-pyrazolo[3,4-b]pyrazine (100 mg, 37%) as a yellow solid. MS m/z: 581 [M+H]⁺.

Step 2: 1-(2,2-difluoroethyl)-3-methoxy-6-(2-(6-methyl-2-(trifluoromethyl)pyrimidin-4-yl)-2,6-diazaspiro[3.4]octan-6-yl)-1H-pyrazolo[3,4-b]pyrazine: Followed the general procedure J using 1-(2,2-difluoroethyl)-3-iodo-6-(2-(6-methyl-2-(trifluoromethyl)pyrimidin-4-yl)-2,6-diazaspiro[3.4]octan-6-yl)-1H-pyrazolo[3,4-b]pyrazine (100 mg, 0.17 μmol, 1.0 equiv.) and sodium 2-methylpropan-2-olate (33 mg, 0.34 μmol, 2.0 equiv.) as the starting materials to give 1-(2,2-difluoroethyl)-3-methoxy-6-(2-(6-methyl-2-(trifluoromethyl)pyrimidin-4-yl)-2,6-diazaspiro[3.4]octan-6-yl)-1H-pyrazolo[3,4-b]pyrazine (46.4 mg, 25%) as a white solid. ¹H NMR (300 MHz, Methanol-d₄) δ 7.87 (d, J=1.7 Hz, 1H), 6.40 (d, J=1.3 Hz, 1H), 6.13 (dt, J=55.7, 4.3 Hz, 1H), 4.49 (td, J=13.7, 4.3 Hz, 2H), 4.25-4.10 (m, 4H), 4.06 (s, 3H), 3.89 (s, 2H), 3.74 (t, J=6.9 Hz, 2H), 2.38 (s, 5H). MS m/z: 485.1[M+H]⁺.

### Example 1098: 8-(1-(2,2-difluoroethyl)-3-methoxy-1H-pyrazolo[3,4-b]pyrazin-6-yl)-2-(6-methyl-2-(trifluoromethyl)pyrimidin-4-yl)-2,8-diazaspiro[4.5]decane

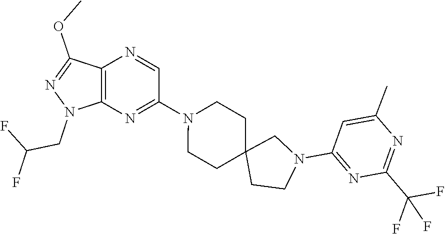 [SMILES: COc1nn(CC(F)F)c2nc(N3CCC4(CCN(c5cc(C)nc(C(F)(F)F)n5)C4)CC3)cnc12]

Step 1: 8-(3-iodo-1-(tetrahydro-2H-pyran-2-yl)-1H-pyrazolo[3,4-b]pyrazin-6-yl)-2-(6-methyl-2-(trifluoromethyl)pyrimidin-4-yl)-2,8-diazaspiro[4.5]decane: Followed the general procedure I using 2-(6-methyl-2-(trifluoromethyl)pyrimidin-4-yl)-2,8-diazaspiro[4.5]decane (100 mg, 333 μmol, 1.0 equiv.) and 6-chloro-3-iodo-1-(tetrahydro-2H-pyran-2-yl)-1H-pyrazolo[3,4-b]pyrazine (121 mg, 333 μmol, 1.0 equiv.) as the starting materials to give 8-(3-iodo-1-(tetrahydro-2H-pyran-2-yl)-1H-pyrazolo[3,4-b]pyrazin-6-yl)-2-(6-methyl-2-(trifluoromethyl)pyrimidin-4-yl)-2,8-diazaspiro[4.5]decane (190 mg, 91%) as a white solid. MS m/z: 629 [M+H]⁺.

Step 2: 8-(3-methoxy-1-(tetrahydro-2H-pyran-2-yl)-1H-pyrazolo[3,4-b]pyrazin-6-yl)-2-(6-methyl-2-(trifluoromethyl)pyrimidin-4-yl)-2,8-diazaspiro[4.5]decane: Followed the general procedure J using 8-(3-iodo-1-(tetrahydro-2H-pyran-2-yl)-1H-pyrazolo[3,4-b]pyrazin-6-yl)-2-(6-methyl-2-(trifluoromethyl)pyrimidin-4-yl)-2,8-diazaspiro[4.5]decane (180 mg, 286 μmol, 1.0 equiv.) and MeOH (0.4 mL) as the starting materials to give 8-(3-methoxy-1-(tetrahydro-2H-pyran-2-yl)-1H-pyrazolo[3,4-b]pyrazin-6-yl)-2-(6-methyl-2-(trifluoromethyl)pyrimidin-4-yl)-2,8-diazaspiro[4.5]decane (140 mg, 92%) as a white solid. MS m/z: 533 [M+H]⁺.

Step 3: 8-(3-methoxy-1H-pyrazolo[3,4-b]pyrazin-6-yl)-2-(6-methyl-2-(trifluoromethyl)pyrimidin-4-yl)-2,8-diazaspiro[4.5]decane: Followed the general procedure M using 8-(3-methoxy-1-(tetrahydro-2H-pyran-2-yl)-1H-pyrazolo[3,4-b]pyrazin-6-yl)-2-(6-methyl-2-(trifluoromethyl)pyrimidin-4-yl)-2,8-diazaspiro[4.5]decane (130 mg, 244 μmol, 1.0 equiv.) as the starting material to give 8-(3-methoxy-1H-pyrazolo[3,4-b]pyrazin-6-yl)-2-(6-methyl-2-(trifluoromethyl)pyrimidin-4-yl)-2,8-diazaspiro[4.5]decane (85 mg, 78%) as a white solid. MS m/z: 449 [M+H]⁺.

Step 4: 8-(1-(2,2-difluoroethyl)-3-methoxy-1H-pyrazolo[3,4-b]pyrazin-6-yl)-2-(6-methyl-2-(trifluoromethyl)pyrimidin-4-yl)-2,8-diazaspiro[4.5]decane: Followed the general procedure K using 8-(3-methoxy-1H-pyrazolo[3,4-b]pyrazin-6-yl)-2-(6-methyl-2-(trifluoromethyl)pyrimidin-4-yl)-2,8-diazaspiro[4.5]decane (85 mg, 190 μmol, 1.0 equiv.) and 2,2-difluoroethyl trifluoromethanesulfonate (84.2 mg, 380 μmol, 2.0 equiv.) as the starting materials to give 8-(1-(2,2-difluoroethyl)-3-methoxy-1H-pyrazolo[3,4-b]pyrazin-6-yl)-2-(6-methyl-2-(trifluoromethyl)pyrimidin-4-yl)-2,8-diazaspiro[4.5]decane (13.6 mg, 14%) as a white solid. ¹H NMR (400 MHz, DMSO-d₆) δ 8.34 (d, J=4.2 Hz, 1H), 6.60 (s, 1H), 6.53-6.14 (m, 1H), 4.62-4.38 (m, 2H), 3.99 (s, 3H), 3.86 (t, J=18.1 Hz, 2H), 3.73 (s, 2H), 3.62 (s, 1H), 3.48 (s, 2H), 3.37 (s, 1H), 2.34 (d, J=4.6 Hz, 3H), 1.95 (d, J=36.6 Hz, 2H), 1.66 (d, J=4.7 Hz, 4H). MS m/z: 513.3 [M+H]⁺.

### Example 1099: 1-(2,2-difluoroethyl)-5-ethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one

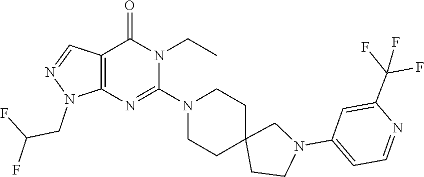 [SMILES: CCn1c(N2CCC3(CCN(c4ccnc(C(F)(F)F)c4)C3)CC2)nc2c(cnn2CC(F)F)c1=O]

Step 1: 5-ethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure I using 6-chloro-5-ethyl-1-(tetrahydro-2H-pyran-2-yl)-1,5-dihydro-4H-pyrazolo[3,4-d]pyrimidin-4-one (100 mg, 0.223 mmol, 1.0 equiv.) and 2-(2-(trifluoromethyl)pyridin-4-yl)-2,8-diazaspiro[4.5]decane (57.4 mg, 0.268 mmol, 1.2 equiv.) as the starting materials to give 5-ethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (100 mg, 84%) as a white solid. MS m/z: 532 [M+H]⁺.

Step 2: 5-ethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure H using tert-butyl 5-ethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (100 mg) as the starting material to give the crude product 5-ethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (80 mg). MS m/z: 448 [M+H]⁺.

Step 3: 1-(2,2-difluoroethyl)-5-ethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure K using 3-iodo-6-(2-(2-(trifluoromethyl)pyridin-4-yl)-2,6-diazaspiro[3.4]octan-6-yl)-1H-pyrazolo[3,4-b]pyrazine (240 mg, 0.479 mmol, 1.0 equiv.) and 2,2-difluoroethyl trifluoromethanesulfonate (112 mg, 0.527 mmol, 1.1 equiv.) as the starting materials to give 1-(2,2-difluoroethyl)-5-ethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (100 mg, 36.9%) as a white solid. ¹H NMR (400 MHz, DMSO-d₆) δ 8.21 (d, J=5.8 Hz, 1H), 8.04 (s, 1H), 6.88 (s, 1H), 6.69 (d, J=6.2 Hz, 1H), 6.54-6.32 (m, 1H), 4.64 (td, J=14.8, 3.8 Hz, 2H), 4.03 (q, J=6.9 Hz, 2H), 3.45 (t, J=7.0 Hz, 2H), 3.36 (s, 2H), 3.26 (s, 2H), 3.23-3.15 (m, 2H), 1.96 (t, J=7.0 Hz, 2H), 1.74 (d, J=5.5 Hz, 4H), 1.21 (t, J=6.9 Hz, 3H). MS m/z: 512.15 [M+H]⁺.

### Example 1100: 1-(2,2-difluoroethyl)-5-ethyl-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one

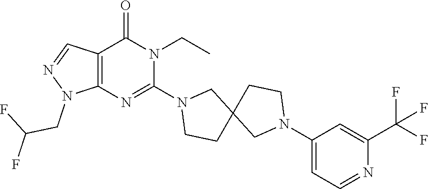 [SMILES: CCn1c(N2CCC3(CCN(c4ccnc(C(F)(F)F)c4)C3)C2)nc2c(cnn2CC(F)F)c1=O]

Step 1: tert-butyl 7-(2-(trifluoromethyl)pyridin-4-yl)-2,7-diazaspiro[4.4]nonane-2-carboxylate: Followed the general procedure I using tert-butyl 2,7-diazaspiro[4.4]nonane-2-carboxylate (250 mg, 1.1 mmol, 1.0 equiv.) and 4-chloro-2-(trifluoromethyl)pyridine (238 mg, 1.32 mmol, 1.2 equiv.) as the starting materials to give tert-butyl 7-(2-(trifluoromethyl)pyridin-4-yl)-2,7-diazaspiro[4.4]nonane-2-carboxylate (315 mg, 76%) as a white solid. MS m/z: 372 [M+H]⁺.

Step 2: 2-(2-(trifluoromethyl)pyridin-4-yl)-2,7-diazaspiro[4.4]nonane: Followed the general procedure H using tert-butyl 7-(2-(trifluoromethyl)pyridin-4-yl)-2,7-diazaspiro[4.4]nonane-2-carboxylate (315 mg, 0.848 mmol, 1.0 equiv.) as the starting material to give the crude product 2-(2-(trifluoromethyl)pyridin-4-yl)-2,7-diazaspiro[4.4]nonane (210 mg), as a white solid. MS m/z: 272 [M+H]⁺.

Step 3: 5-ethyl-1-(tetrahydro-2H-pyran-2-yl)-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one: Followed the general procedure I using 2-(2-(trifluoromethyl)pyridin-4-yl)-2,7-diazaspiro[4.4]nonane (210 mg, 0.772 mmol, 1.0 equiv.) and 6-chloro-5-ethyl-1-(tetrahydro-2H-pyran-2-yl)-1,5-dihydro-4H-pyrazolo[3,4-d]pyrimidin-4-one (263 mg, 0.927 mmol, 1.2 equiv.) as the starting materials to give 5-ethyl-1-(tetrahydro-2H-pyran-2-yl)-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one (280 mg, 70%) as a white solid. MS m/z: 518 [M+H]⁺.

Step 4: 5-ethyl-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one: Followed the general procedure M using 5-ethyl-1-(tetrahydro-2H-pyran-2-yl)-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one (280 mg, 0.541 mmol, 1.0 equiv.) as the starting material to give 5-ethyl-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one (180 mg, 76%) as a white solid. MS m/z: 434 [M+H]⁺.

Step 5: 1-(2,2-difluoroethyl)-5-ethyl-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one: Followed the general procedure K using 5-ethyl-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one (180 mg, 0.415 mmol, 1.0 equiv.) and 2,2-difluoroethyl trifluoromethanesulfonate (98.1 mg, 0.456 mmol, 1.1 equiv.) as the starting materials to give 1-(2,2-difluoroethyl)-5-ethyl-6-(7-(2-(trifluoromethyl)pyridin-4-yl)-2,7-diazaspiro[4.4]nonan-2-yl)-1,5-dihydro-4H-pyrazolo[3,4-d]pyrimidin-4-one (25.1 mg, 12%) as a yellow solid. MS m/z: 498.15 [M+H]⁺. ¹H NMR (400 MHz, DMSO-d₆) δ 8.22 (d, J=5.8 Hz, 1H), 7.94 (s, 1H), 6.83 (d, J=2.1 Hz, 1H), 6.67 (d, J=6.3 Hz, 1H), 6.57-6.26 (m, 1H), 4.56 (td, J=14.9, 4.1 Hz, 2H), 4.06 (d, J=10.9 Hz, 2H), 3.74 (t, J=6.8 Hz, 2H), 3.59 (s, 2H), 3.49 (t, J=6.8 Hz, 3H), 3.44-3.39 (m, 1H), 2.09-1.97 (m, 4H), 1.26 (t, J=6.9 Hz, 3H). MS m/z: 498.15 [M+H]⁺.

### Example 1101: 1-(2,2-difluoroethyl)-5-ethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one

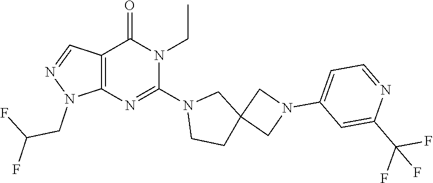 [SMILES: CCn1c(N2CCC3(CN(c4ccnc(C(F)(F)F)c4)C3)C2)nc2c(cnn2CC(F)F)c1=O]

Step 1: 5-ethyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure I using 6-chloro-5-ethyl-1-(oxan-2-yl)pyrazolo[3,4-d]pyrimidin-4-one (100 mg, 0.354 mmol, 1.0 equiv.) and 2-[2-(trifluoromethyl)pyridin-4-yl]-2,6-diazaspiro[3.4]octane (110 mg, 0.425 mmol, 1.2 equiv.) as the starting materials to give 5-ethyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (100 mg, 55%) as a white solid. MS m/z: 504 [M+H]⁺.

Step 2: 5-ethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure M using tert-butyl 5-ethyl-1-(tetrahydro-2H-pyran-2-yl)-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (100 mg, 0.199 mmol, 1.0 equiv.) as the starting material to give 5-ethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (80 mg, 96%) as a white solid. MS m/z: 420 [M+H]⁺.

Step 3: 1-(2,2-difluoroethyl)-5-ethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one: Followed the general procedure K using 5-ethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (80 mg, 0.190 mmol, 1.0 equiv.) and 2,2-difluoroethyl trifluoromethanesulfonate (50 mg, 0.228 mmol, 1.2 equiv.) as the starting materials to give 1-(2,2-difluoroethyl)-5-ethyl-6-(2-(2-(trifluoromethyl)pyridin-4-yl)-2,6-diazaspiro[3.4]octan-6-yl)-1,5-dihydro-4H-pyrazolo[3,4-d]pyrimidin-4-one (49 mg, 53%) as a white solid. ¹H NMR (400 MHz, DMSO-d₆) δ 8.24 (d, J=5.7 Hz, 1H), 7.95 (s, 1H), 6.75 (d, J=2.2 Hz, 1H), 6.58-6.29 (m, 2H), 4.57 (td, J=14.8, 3.9 Hz, 2H), 4.09-3.96 (m, 6H), 3.81 (s, 2H), 3.65 (t, J=6.7 Hz, 2H), 2.22 (t, J=6.8 Hz, 2H), 1.26 (t, J=7.0 Hz, 3H). MS m/z: 484.10 [M+H]⁺.

### Example 1102: 1-(2,2-difluoroethyl)-5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one

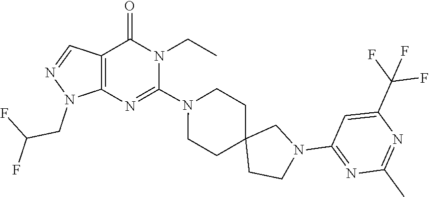 [SMILES: CCn1c(N2CCC3(CCN(c4cc(C(F)(F)F)nc(C)n4)C3)CC2)nc2c(cnn2CC(F)F)c1=O]

Step 1: 5-ethyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one: Followed the general procedure I using 6-chloro-5-ethyl-1-(tetrahydro-2H-pyran-2-yl)-1,5-dihydro-4H-pyrazolo[3,4-d]pyrimidin-4-one (110 mg, 0.389 mmol, 1.0 equiv.) and 2-(2-methyl-6-(trifluoromethyl)pyrimidin-4-yl)-2,8-diazaspiro[4.5]decane (128 mg, 0.428 mmol, 1.1 equiv.) as the starting materials to give 5-ethyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (100 mg, 43%) as a white solid. MS m/z: 547 [M+H]⁺.

Step 2: 5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure M using 5-ethyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (100 mg, 0.183 mmol, 1.0 equiv.) as the starting material to give 5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (100 mg, 90%) as a brown oil solid. MS m/z: 463 [M+H]⁺.

Step 3: 1-(2,2-difluoroethyl)-5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure K using 5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (100 mg, 0.216 mmol, 1.0 equiv.) and 2,2-difluoroethyl trifluoromethanesulfonate (56 mg, 0.259 mmol, 1.2 equiv.) as the starting materials to give 1-(2,2-difluoroethyl)-5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (100 mg, 36%) as a white solid. ¹H NMR (400 MHz, DMSO-d₆) δ 8.04 (s, 1H), 6.76 (d, J=44.6 Hz, 1H), 6.63-6.16 (m, 1H), 6.65-6.25 (m, 1H), 4.05-3.98 (m, 2H), 3.63 (d, J=7.6 Hz, 1H), 3.53 (d, J=10.7 Hz, 2H), 3.43 (s, 1H), 3.18 (s, 4H), 2.43 (s, 3H), 1.98 (s, 1H), 1.90 (t, J=6.9 Hz, 1H), 1.73 (s, 4H), 1.21 (t, J=6.9 Hz, 3H). MS m/z: 527.15 [M+H]⁺.

### Example 1103: 1-(2,2-difluoroethyl)-5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one

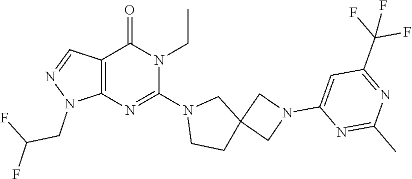 [SMILES: CCn1c(N2CCC3(CN(c4cc(C(F)(F)F)nc(C)n4)C3)C2)nc2c(cnn2CC(F)F)c1=O]

Step 1: 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure I using 6-chloro-5-ethyl-1-(tetrahydro-2H-pyran-2-yl)-1,5-dihydro-4H-pyrazolo[3,4-d]pyrimidin-4-one (110 mg, 0.389 mmol, 1.0 equiv.) and 2-(2-methyl-6-(trifluoromethyl)pyrimidin-4-yl)-2,6-diazaspiro[3.4]octane (117 mg, 0.428 mmol, 1.1 equiv.) as the starting materials to give 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (106 mg, 43%) as a light brown oil. MS m/z: 519 [M+H]⁺.

Step 2: 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure M using 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (106 mg, 0.183 mmol, 1.0 equiv.) as the starting material to give 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (100 mg, 92%) as a brown oil. MS m/z: 435 [M+H]⁺.

Step 3: 1-(2,2-difluoroethyl)-5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure K using 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (100 mg, 0.230 mmol, 1.0 equiv.) and 2,2-difluoroethyl trifluoromethanesulfonate (60 mg, 0.276 mmol, 1.2 equiv.) as the starting materials to give 1-(2,2-difluoroethyl)-5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (28.3 mg, 36%) as a white solid. ¹H NMR (400 MHz, DMSO-d₆) δ 7.94 (s, 1H), 6.75-6.55 (m, 1H), 6.52-6.13 (m, 1H), 4.66-4.47 (m, 2H), 4.16 (d, J=9.2 Hz, 2H), 4.14-3.97 (m, 4H), 3.80 (s, 2H), 3.65 (t, J=6.8 Hz, 2H), 2.42 (s, 3H), 2.21 (t, J=6.9 Hz, 2H), 1.25 (t, J=6.9 Hz, 3H). MS m/z: 499 [M+H]⁺.

### Example 1104: 1-(2,2-difluoroethyl)-3,5-dimethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one

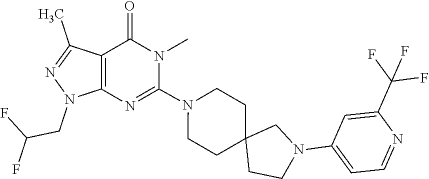 [SMILES: Cc1nn(CC(F)F)c2nc(N3CCC4(CCN(c5ccnc(C(F)(F)F)c5)C4)CC3)n(C)c(=O)c12]

Step 1: tert-butyl 2-(2-(trifluoromethyl)pyridin-4-yl)-2,8-diazaspiro[4.5]decane-8-carboxylate: Followed the general procedure I using tert-butyl 2,8-diazaspiro[4.5]decane-8-carboxylate (200 mg, 0.832 mmol, 1.0 equiv.) and 4-chloro-2-(trifluoromethyl)pyridine (181 mg, 0.998 mmol, 1.2 equiv.) as the starting materials to give tert-butyl 2-(2-(trifluoromethyl)pyridin-4-yl)-2,8-diazaspiro[4.5]decane-8-carboxylate (195 mg, 60%) as a white solid. MS m/z: 385 [M+H]⁺

Step 2: 2-(2-(trifluoromethyl)pyridin-4-yl)-2,8-diazaspiro[4.5]decane hydrochloride: Followed the general procedure B using tert-butyl 2-(2-(trifluoromethyl)pyridin-4-yl)-2,8-diazaspiro[4.5]decane-8-carboxylate (175 mg, 0.454 mmol, 1 equiv.) as the starting material to give the crude product 2-(2-(trifluoromethyl)pyridin-4-yl)-2,8-diazaspiro[4.5]decane hydrochloride (130 mg) was used for next step without further purification. MS m/z: 285 [M+H]⁺.

Step 3: 3-iodo-5-methyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure I using 2-(2-(trifluoromethyl)pyridin-4-yl)-2,8-diazaspiro[4.5]decane hydrochloride (130 mg, 0.456 mmol, 1.0 equiv.) and 6-chloro-3-iodo-5-methyl-1-(tetrahydro-2H-pyran-2-yl)-1,5-dihydro-4H-pyrazolo[3,4-d]pyrimidin-4-one (197 mg, 0.502 mmol, 1.1 equiv.) as the starting materials to give 3-iodo-5-methyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (280 mg, 95%) as a white solid. MS m/z: 643 [M+H]⁺.

Step 4: 3,5-dimethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure L using 3-iodo-5-methyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (260 mg, 0.404 mmol, 1.0 equiv.) and 2,4,6-trimethyl-1,3,5,2,4,6-trioxatriborinane (152 mg, 1.21 mmol, 3.0 equiv.) as the starting materials to give 3,5-dimethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (170 mg, 79%) as a yellow oil. MS m/z: 531 [M+H]⁺.

Step 5: 3,5-dimethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one: Followed the general procedure M using 3,5-dimethyl-1-(tetrahydro-2H-pyran-2-yl)-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (170 mg, 0.32 mmol, 1.0 equiv.) as the starting material to give 3,5-dimethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (140 mg, 98%) as a white solid. MS m/z: 447 [M+H]⁺.

Step 6: 8-(1-(2,2-difluoroethyl)-3-ethoxy-1H-pyrazolo[3,4-b]pyrazin-6-yl)-2-(6-(trifluoromethyl)pyridin-3-yl)-2,8-diazaspiro[4.5]decan-3-one: Followed the general procedure K using 3,5-dimethyl-6-(2-(2-(trifluoromethyl)pyridin-4-yl)-2,8-diazaspiro[4.5]decan-8-yl)-1,5-dihydro-4H-pyrazolo[3,4-d]pyrimidin-4-one (80 mg, 0.179 mmol, 1.0 equiv.) and 2,2-difluoroethyl trifluoromethanesulfonate (57.4 mg, 0.268 mmol, 1.5 equiv.) as the starting materials to give 8-(1-(2,2-difluoroethyl)-3-ethoxy-1H-pyrazolo[3,4-b]pyrazin-6-yl)-2-(6-(trifluoromethyl)pyridin-3-yl)-2,8-diazaspiro[4.5]decan-3-one (45.6 mg, 49%) as a white solid. ¹H NMR (400 MHz, DMSO-d₆) δ 8.21 (d, J=5.8 Hz, 1H), 6.88 (s, 1H), 6.69 (d, J=5.4 Hz, 1H), 6.56-6.21 (m, 1H), 4.58-4.49 (m, 2H), 3.49-3.42 (m, 4H), 3.40 (s, 3H) 3.26-3.17 (m, 4H), 2.39 (s, 3H), 1.96 (t, J=7.0 Hz, 2H), 1.72 (t, J=5.6 Hz, 4H). MS m/z: 512.05[M+H]⁺.

### Example 1105: 1-(2,2-difluoroethyl)-5-ethyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one

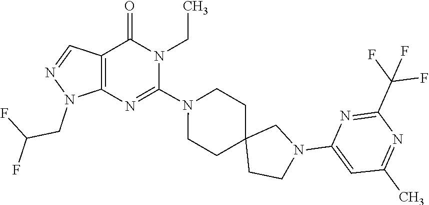 [SMILES: CCn1c(N2CCC3(CCN(c4cc(C)nc(C(F)(F)F)n4)C3)CC2)nc2c(cnn2CC(F)F)c1=O]

Step 1: 5-ethyl-6-(2-(6-methyl-2-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one: Followed the general procedure I using 2-(6-methyl-2-(trifluoromethyl)pyrimidin-4-yl)-2,8-diazaspiro[4.5]decane (110 mg, 366 μmol, 1.0 equiv.) and 6-chloro-5-ethyl-1-(tetrahydro-2H-pyran-2-yl)-1,5-dihydro-4H-pyrazolo[3,4-d]pyrimidin-4-one (103 mg, 366 μmol, 1.0 equiv.) as the starting materials to give 5-ethyl-6-(2-(6-methyl-2-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (180 mg, 90%) as a white solid. MS m/z: 547 [M+H]⁺.

Step 2: 5-ethyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure M using 5-ethyl-6-(2-(6-methyl-2-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (170 mg, 311 μmol, 1.0 equiv.) as the starting material to give 5-ethyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one (140 mg, 97%) as a white solid. MS m/z: 463 [M+H]⁺.

Step 3: 1-(2,2-difluoroethyl)-5-ethyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure K using 5-ethyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one (140 mg, 303 μmol, 1.0 equiv.) and 2,2-difluoroethyl trifluoromethanesulfonate (129 mg, 606 μmol, 2.0 equiv.) as the starting materials to give 1-(2,2-difluoroethyl)-5-ethyl-6-(2-(6-methyl-2-(trifluoromethyl)pyrimidin-4-yl)-2,8-diazaspiro[4.5]decan-8-yl)-1,5-dihydro-4H-pyrazolo[3,4-d]pyrimidin-4-one (58.8 mg, 37%) as a white solid. ¹H NMR (400 MHz, DMSO-d₆) δ 8.04 (d, J=2.0 Hz, 1H), 6.71-6.26 (m, 2H), 4.64 (td, J=14.9, 3.8 Hz, 2H), 4.03 (q, J=6.9 Hz, 2H), 3.62 (t, J=6.9 Hz, 1H), 3.50 (d, J=6.8 Hz, 2H), 3.37 (s, 1H), 3.19 (d, J=7.3 Hz, 4H), 2.34 (s, 3H), 1.95 (dt, J=35.6, 7.2 Hz, 2H), 1.74 (s, 4H), 1.21 (t, J=6.9 Hz, 3H). MS m/z: 527.1 [M+H]⁺.

### Example 1106: 1-(2,2-difluoroethyl)-6-(2-(2-methyl-6-(trifluoromethyl)pyrimidin-4-yl)-2,8-diazaspiro[4.5]decan-8-yl)-5-propyl-1,5-dihydro-4H-pyrazolo[3,4-d]pyrimidin-4-one

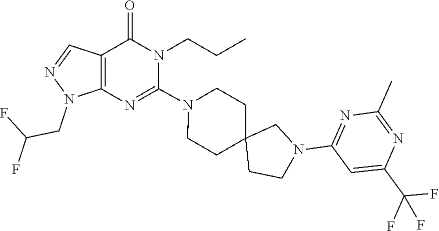 [SMILES: CCCn1c(N2CCC3(CCN(c4cc(C(F)(F)F)nc(C)n4)C3)CC2)nc2c(cnn2CC(F)F)c1=O]

Step 1: 6-chloro-1-(tetrahydro-2H-pyran-2-yl)-5-((2-(trimethylsilyl)ethoxy)methyl)-1,5-dihydro-4H-pyrazolo[3,4-d]pyrimidin-4-one: Followed the general procedure R using 6-chloro-1-(tetrahydro-2H-pyran-2-yl)-1,5-dihydro-4H-pyrazolo[3,4-d]pyrimidin-4-one (1000 mg, 3.92 mmol, 1.0 equiv.) and SEMCl (654 mg, 3.92 mmol, 1.0 equiv.) as the starting materials to give 6-chloro-1-(tetrahydro-2H-pyran-2-yl)-5-((2-(trimethylsilyl)ethoxy)methyl)-1,5-dihydro-4H-pyrazolo[3,4-d]pyrimidin-4-one (800 mg, 53%) as a white solid. MS m/z: 385 [M+H]⁺.

Step 2: 6-(2-(2-methyl-6-(trifluoromethyl)pyrimidin-4-yl)-2,8-diazaspiro[4.5]decan-8-yl)-1-(tetrahydro-2H-pyran-2-yl)-5-((2-(trimethylsilyl)ethoxy)methyl)-1,5-dihydro-4H-pyrazolo[3,4-d]pyrimidin-4-one: Followed the general procedure I using 6-chloro-1-(tetrahydro-2H-pyran-2-yl)-5-((2-(trimethylsilyl)ethoxy)methyl)-1,5-dihydro-4H-pyrazolo[3,4-d]pyrimidin-4-one (800 mg, 2.07 mmol) and 2-(2-methyl-6-(trifluoromethyl)pyrimidin-4-yl)-2,8-diazaspiro[4.5]decane (625 mg, 2.07 mmol, 1.0 equiv.) as the starting materials to give 6-(2-(2-methyl-6-(trifluoromethyl)pyrimidin-4-yl)-2,8-diazaspiro[4.5]decan-8-yl)-1-(tetrahydro-2H-pyran-2-yl)-5-((2-(trimethylsilyl)ethoxy)methyl)-1,5-dihydro-4H-pyrazolo[3,4-d]pyrimidin-4-one (650 mg, 48%) as a white solid. MS m/z: 649 [M+H]⁺.

Step 3: 6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one: Followed the general procedure AG using 6-(2-(2-methyl-6-(trifluoromethyl)pyrimidin-4-yl)-2,8-diazaspiro[4.5]decan-8-yl)-1-(tetrahydro-2H-pyran-2-yl)-5-((2-(trimethylsilyl)ethoxy)methyl)-1,5-dihydro-4H-pyrazolo[3,4-d]pyrimidin-4-one (650 mg, 1.00 mmol, 1.0 equiv.) as the starting material to give 6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (400 mg, 77%) as a colorless oil. MS m/z: 519 [M+H]⁺

Step 4: 6-(2-(2-methyl-6-(trifluoromethyl)pyrimidin-4-yl)-2,8-diazaspiro[4.5]decan-8-yl)-5-propyl-1-(tetrahydro-2H-pyran-2-yl)-1,5-dihydro-4H-pyrazolo[3,4-d]pyrimidin-4-one: Followed the general procedure K using 6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (200 mg, 385 μmol, 1.0 equiv.) and 1-iodopropane (66 mg, 385 μmol, 1.0 equiv.) as the starting materials to give 6-(2-(2-methyl-6-(trifluoromethyl)pyrimidin-4-yl)-2,8-diazaspiro[4.5]decan-8-yl)-5-propyl-1-(tetrahydro-2H-pyran-2-yl)-1,5-dihydro-4H-pyrazolo[3,4-d]pyrimidin-4-one (150 mg, 69%) as a white solid. MS m/z: 561 [M+H]⁺.

Step 5: 6-(2-methyl-6-(trifluoromethyl)pyrimidin-4-yl)-2,8-diazaspiro[4.5]decan-8-yl)-5-propyl-1,5-dihydro-4H-pyrazolo[3,4-d]pyrimidin-4-one: Followed the general procedure M using 6-(2-(2-methyl-6-(trifluoromethyl)pyrimidin-4-yl)-2,8-diazaspiro[4.5]decan-8-yl)-5-propyl-1-(tetrahydro-2H-pyran-2-yl)-1,5-dihydro-4H-pyrazolo[3,4-d]pyrimidin-4-one (150 mg, 267 μmol, 1.0 equiv.) as the starting material to give 6-(2-(2-methyl-6-(trifluoromethyl)pyrimidin-4-yl)-2,8-diazaspiro[4.5]decan-8-yl)-5-propyl-1,5-dihydro-4H-pyrazolo[3,4-d]pyrimidin-4-one (120 mg, 93%) as a white solid. MS m/z: 477 [M+H]⁺.

Step 6: 1-(2,2-difluoroethyl)-6-(2-(2-methyl-6-(trifluoromethyl)pyrimidin-4-yl)-2,8-diazaspiro[4.5]decan-8-yl)-5-propyl-1,5-dihydro-4H-pyrazolo[3,4-d]pyrimidin-4-one: Followed the general procedure K using 6-(2-(2-methyl-6-(trifluoromethyl)pyrimidin-4-yl)-2,8-diazaspiro[4.5]decan-8-yl)-5-propyl-1,5-dihydro-4H-pyrazolo[3,4-d]pyrimidin-4-one (120 mg, 251 μmol, 1.0 equiv.) and 2,2-difluoroethyl trifluoromethanesulfonate (55 mg, 251 μmol, 1.0 equiv.) as the starting materials to give 1-(2,2-difluoroethyl)-6-(2-(2-methyl-6-(trifluoromethyl)pyrimidin-4-yl)-2,8-diazaspiro[4.5]decan-8-yl)-5-propyl-1,5-dihydro-4H-pyrazolo[3,4-d]pyrimidin-4-one (48 mg, 92%) as a white solid. ¹H NMR (400 MHz, DMSO-d₆) δ 7.93 (s, 1H), 6.74 (d, J=20.4 Hz, 1H), 6.57-6.24 (m, 1H), 4.62 (td, J=14.8, 3.7 Hz, 2H), 4.42 (t, J=6.6 Hz, 2H), 4.01 (d, J=12.9 Hz, 1H), 3.87 (s, 2H), 3.78-3.37 (m, 5H), 2.43 (d, J=1.6 Hz, 3H), 2.09-1.73 (m, 4H), 1.60 (d, J=6.7 Hz, 4H), 0.98 (t, J=7.4 Hz, 3H). MS m/z: 541.20 [M+H]⁺.

### Example 1107: 1-(2,2-difluoroethyl)-5-isobutyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one

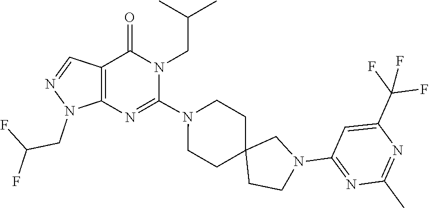 [SMILES: Cc1nc(N2CCC3(CCN(c4nc5c(cnn5CC(F)F)c(=O)n4CC(C)C)CC3)C2)cc(C(F)(F)F)n1]

Step 1: 5-isobutyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one: Followed the general procedure K using 6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (200 mg, 385 μmol, 1.0 equiv.) and 1-iodo-2-methylpropane (72 mg, 385 μmol, 1.0 equiv.) as the starting materials to give 5-isobutyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (150 mg, 67%) as a white solid. MS m/z: 575 [M+H]⁺.

Step 2: 5-isobutyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure M using 5-isobutyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (150 mg, 260 μmol, 1.0 equiv.) as the starting material to give 5-isobutyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (120 mg, 93%) as a white solid. MS m/z: 491 [M+H]⁺.

Step 3: 1-(2,2-difluoroethyl)-5-isobutyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure K using 5-isobutyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (120 mg, 244 μmol) and 2,2-difluoroethyl trifluoromethanesulfonate (53 mg, 244 μmol, 1.0 equiv.) as the starting materials to give 1-(2,2-difluoroethyl)-5-isobutyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (48 mg, 35%) as a white solid. ¹H NMR (400 MHz, DMSO-d₆) δ 7.95 (s, 1H), 6.74 (d, J=20.6 Hz, 1H), 6.56-6.26 (m, 1H), 4.62 (td, J=14.8, 3.8 Hz, 2H), 4.25 (d, J=6.5 Hz, 2H), 4.00 (d, J=11.8 Hz, 1H), 3.86 (s, 2H), 3.64 (tt, J=42.7, 7.3 Hz, 5H), 2.43 (d, J=1.6 Hz, 3H), 2.11 (tt, J=13.3, 6.9 Hz, 1H), 1.92 (dt, J=34.2, 7.0 Hz, 2H), 1.61 (t, J=6.1 Hz, 4H), 0.99 (d, J=6.7 Hz, 6H). MS m/z: 555.35 [M+H]⁺.

### Example 1108: 5-(cyclopropylmethyl)-1-(2,2-difluoroethyl)-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one

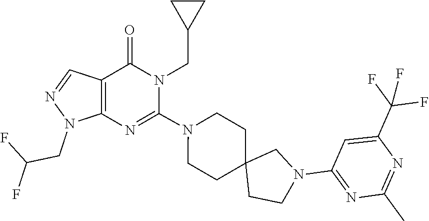 [SMILES: Cc1nc(N2CCC3(CCN(c4nc5c(cnn5CC(F)F)c(=O)n4CC4CC4)CC3)C2)cc(C(F)(F)F)n1]

Step 1: 6-chloro-5-(cyclopropylmethyl)-1-(tetrahydro-2H-pyran-2-yl)-1,5-dihydro-4H-pyrazolo[3,4-d]pyrimidin-4-one: Followed the general procedure K using 6-chloro-1-(tetrahydro-2H-pyran-2-yl)-1,5-dihydro-4H-pyrazolo[3,4-d]pyrimidin-4-one (500 mg, 1.96 mmol, 1.0 equiv.) and (iodomethyl)cyclopropane (431 mg, 2.36 mmol, 1.2 equiv.) as the starting materials to give 6-chloro-5-(cyclopropylmethyl)-1-(tetrahydro-2H-pyran-2-yl)-1,5-dihydro-4H-pyrazolo[3,4-d]pyrimidin-4-one (240 mg, 39%) as a white solid. MS m/z: 309 [M+H]⁺.

Step 2: 5-(cyclopropylmethyl)-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one: Followed the general procedure I using 6-chloro-5-(cyclopropylmethyl)-1-(tetrahydro-2H-pyran-2-yl)-1,5-dihydro-4H-pyrazolo[3,4-d]pyrimidin-4-one (240 mg, 0.777 mmol, 1.0 equiv.) and 2-(2-methyl-6-(trifluoromethyl)pyrimidin-4-yl)-2,8-diazaspiro[4.5]decane (281 mg, 0.932 mmol, 1.2 equiv.) as the starting materials to give 5-(cyclopropylmethyl)-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (145 mg, 32%) as a white solid. MS m/z: 573 [M+H]⁺.

Step 3: 5-(cyclopropylmethyl)-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure M using 5-(cyclopropylmethyl)-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one[3,4-d]pyrimidin-4-one (145 mg, 0.253 mmol, 1.0 equiv.) as the starting material to give 5-(cyclopropylmethyl)-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (100 mg, 80%) as a white solid. MS m/z: 489 [M+H]⁺.

Step 4: 5-(cyclopropylmethyl)-1-(2,2-difluoroethyl)-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure K using 5-(cyclopropylmethyl)-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (100 mg, 0.205 mmol, 1.0 equiv.) and 2,2-difluoroethyl trifluoromethanesulfonate (52.8 mg, 0.246 mmol, 1.2 equiv.) as the starting materials to give 5-(cyclopropylmethyl)-1-(2,2-difluoroethyl)-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (38.2 mg, 33%) as a white solid. ¹H NMR (400 MHz, DMSO-d₆) δ 8.06 (s, 1H), 6.76 (d, J=44.2 Hz, 1H), 6.47 (tt, J=54.7, 3.7 Hz, 1H), 4.72-4.60 (m, 2H), 3.89 (d, J=6.9 Hz, 2H), 3.64 (t, J=7.1 Hz, 1H), 3.52 (d, J=10.2 Hz, 2H), 3.42 (s, 1H), 3.27-3.09 (m, 4H), 2.43 (s, 3H), 1.93 (dt, J=32.9, 7.1 Hz, 2H), 1.69 (s, 4H), 1.24 (s, 1H), 0.41 (d, J=7.8 Hz, 2H), 0.30 (s, 2H). MS m/z: 553.25 [M+H]⁺.

### Example 1109: 5-ethyl-6-(2-(2-methyl-6-(trifluoromethyl)pyrimidin-4-yl)-2,8-diazaspiro[4.5]decan-8-yl)-1-(2,2,2-trifluoroethyl)-1,5-dihydro-4H-pyrazolo[3,4-d]pyrimidin-4-one

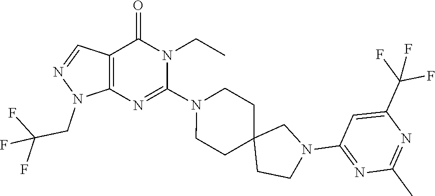 [SMILES: CCn1c(N2CCC3(CCN(c4cc(C(F)(F)F)nc(C)n4)C3)CC2)nc2c(cnn2CC(F)(F)F)c1=O]

Step 1: 5-ethyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one: Followed the general procedure I using 2-[2-methyl-6-(trifluoromethyl)pyrimidin-4-yl]-2,8-diazaspiro[4.5]decane (400 mg, 1.33 mmol, 1.0 equiv.) and 6-chloro-5-ethyl-1-(oxan-2-yl)pyrazolo[3,4-d]pyrimidin-4-one (452 mg, 1.59 mmol, 1.2 equiv.) as the starting materials to give 5-ethyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (240 mg, 43.0%) as a white solid. MS m/z: 547 [M+H]⁺.

Step 2: 5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one: Followed the general procedure M using 5-ethyl-6-(2-(2-methyl-6-(trifluoromethyl)pyrimidin-4-yl)-2,8-diazaspiro[4.5]decan-8-yl)-1-(tetrahydro-2H-pyran-2-yl)-1,5-dihydro-4H-pyrazolo[3,4-d]pyrimidin-4-one (340 mg, 0.622 mmol, 1.0 equiv.) as the starting material to give 5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (240 mg, 83%) as a white solid. MS m/z: 463 [M+H]⁺.

Step 3: 5-ethyl-6-(2-(2-methyl-6-(trifluoromethyl)pyrimidin-4-yl)-2,8-diazaspiro[4.5]decan-8-yl)-1-(2,2,2-trifluoroethyl)-1,5-dihydro-4H-pyrazolo[3,4-d]pyrimidin-4-one: Followed the general procedure K using 5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (80 mg, 0.173 mmol, 1.0 equiv.) and 2,2,2-trifluoroethyl trifluoromethanesulfonate (48 mg, 0.208 mmol, 1.2 equiv.) as the starting materials to give 5-ethyl-6-(2-(2-methyl-6-(trifluoromethyl)pyrimidin-4-yl)-2,8-diazaspiro[4.5]decan-8-yl)-1-(2,2,2-trifluoroethyl)-1,5-dihydro-4H-pyrazolo[3,4-d]pyrimidin-4-one (19 mg, 21%) as a white solid. ¹H NMR (400 MHz, DMSO-d₆) δ 8.09 (s, 1H), 6.76 (d, J=41.9 Hz, 1H), 5.09 (q, J=8.6 Hz, 2H), 4.02 (q, J=6.9 Hz, 2H), 3.72-3.58 (m, 2H), 3.55 (d, J=6.8 Hz, 2H), 3.19 (d, J=6.5 Hz, 4H), 2.43 (s, 3H), 1.98 (t, J=7.0 Hz, 1H), 1.89 (t, J=7.2 Hz, 1H), 1.73 (s, 4H), 1.22 (t, J=6.9 Hz, 3H). MS m/z: 512.15 [M+H]⁺.

### Example 1110: 5-ethyl-6-(2-(2-methyl-6-(trifluoromethyl)pyrimidin-4-yl)-2,8-diazaspiro[4.5]decan-8-yl)-1-(oxetan-3-yl)-1,5-dihydro-4H-pyrazolo[3,4-d]pyrimidin-4-one

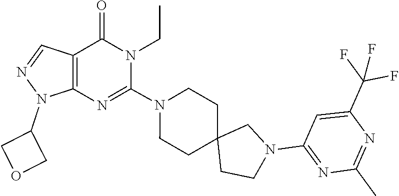 [SMILES: CCn1c(N2CCC3(CCN(c4cc(C(F)(F)F)nc(C)n4)C3)CC2)nc2c(cnn2C2COC2)c1=O]

Step 1: 5-ethyl-6-(2-(2-methyl-6-(trifluoromethyl)pyrimidin-4-yl)-2,8-diazaspiro[4.5]decan-8-yl)-1-(oxetan-3-yl)-1,5-dihydro-4H-pyrazolo[3,4-d]pyrimidin-4-one: Followed the general procedure K using 5-ethyl-6-(2-(2-methyl-6-(trifluoromethyl)pyrimidin-4-yl)-2,8-diazaspiro[4.5]decan-8-yl)-1,5-dihydro-4H-pyrazolo[3,4-d]pyrimidin-4-one (80 mg, 0.173 mmol, 1.0 equiv.) and oxetan-3-yl trifluoromethanesulfonate (42 mg, 0.208 mmol, 1.2 equiv.) as the starting materials to give 5-ethyl-6-(2-(2-methyl-6-(trifluoromethyl)pyrimidin-4-yl)-2,8-diazaspiro[4.5]decan-8-yl)-1-(oxetan-3-yl)-1,5-dihydro-4H-pyrazolo[3,4-d]pyrimidin-4-one (11 mg, 12%) as a white solid. ¹H NMR (400 MHz, DMSO-d₆) δ 8.10 (s, 1H), 6.76 (d, J=45.1 Hz, 1H), 5.81-5.62 (m, J=7.0 Hz, 1H), 4.97 (dt, J=18.5, 6.6 Hz, 4H), 4.02 (q, J=6.8 Hz, 2H), 3.67-3.48 (m, 4H), 3.20 (d, J=35.2 Hz, 4H), 2.43 (s, 3H), 1.98 (t, J=6.9 Hz, 1H), 1.89 (t, J=7.3 Hz, 1H), 1.72 (s, 4H), 1.18 (t, J=6.9 Hz, 3H). MS m/z: 519.15 [M+H]⁺.

### Example 1111: 1-(2,2-difluoroethyl)-6-(2-(2-methyl-6-(trifluoromethyl)pyrimidin-4-yl)-2,6-diazaspiro[3.4]octan-6-yl)-5-propyl-1,5-dihydro-4H-pyrazolo[3,4-d]pyrimidin-4-one

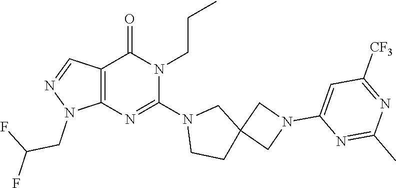 [SMILES: CCCn1c(N2CCC3(CN(c4cc(C(F)(F)F)nc(C)n4)C3)C2)nc2c(cnn2CC(F)F)c1=O]

Step 1: 6-(2-(2-methyl-6-(trifluoromethyl)pyrimidin-4-yl)-2,6-diazaspiro[3.4]octan-6-yl)-1-(tetrahydro-2H-pyran-2-yl)-5-((2-(trimethylsilyl)ethoxy)methyl)-1,5-dihydro-4H-pyrazolo[3,4-d]pyrimidin-4-one: Followed the general procedure I using 6-chloro-1-(tetrahydro-2H-pyran-2-yl)-5-((2-(trimethylsilyl)ethoxy)methyl)-1,5-dihydro-4H-pyrazolo[3,4-d]pyrimidin-4-one (600 mg, 1.55 mmol, 1.0 equiv.) and 2-(2-methyl-6-(trifluoromethyl)pyrimidin-4-yl)-2,6-diazaspiro[3.4]octane (509 mg, 1.87 mmol, 1.2 equiv.) as the starting materials to give 6-(2-(2-methyl-6-(trifluoromethyl)pyrimidin-4-yl)-2,6-diazaspiro[3.4]octan-6-yl)-1-(tetrahydro-2H-pyran-2-yl)-5-((2-(trimethylsilyl)ethoxy)methyl)-1,5-dihydro-4H-pyrazolo[3,4-d]pyrimidin-4-one (610 mg, 60%) as an off-white oil. MS m/z: 621 [M+H]⁺.

Step 2: 6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure AG using 6-(2-(2-methyl-6-(trifluoromethyl)pyrimidin-4-yl)-2,6-diazaspiro[3.4]octan-6-yl)-1-(tetrahydro-2H-pyran-2-yl)-5-((2-(trimethylsilyl)ethoxy)methyl)-1,5-dihydro-4H-pyrazolo[3,4-d]pyrimidin-4-one (600 mg, 0.967 mmol, 1.0 equiv.) as the starting material to give 6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (360 mg, 34%) as a colorless oil. MS m/z: 491 [M+H]⁺.

Step 3: 6-(2-(2-methyl-6-(trifluoromethyl)pyrimidin-4-yl)-2,6-diazaspiro[3.4]octan-6-yl)-5-propyl-1-(tetrahydro-2H-pyran-2-yl)-1,5-dihydro-4H-pyrazolo[3,4-d]pyrimidin-4-one: Followed the general procedure K using 6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (170 mg, 0.347 mmol, 1.0 equiv.) and 1-iodopropane (71 mg, 0.416 mmol, 1.2 equiv.) as the starting materials to give 6-(2-(2-methyl-6-(trifluoromethyl)pyrimidin-4-yl)-2,6-diazaspiro[3.4]octan-6-yl)-5-propyl-1-(tetrahydro-2H-pyran-2-yl)-1,5-dihydro-4H-pyrazolo[3,4-d]pyrimidin-4-one (90 mg, 46%) as a light yellow oil. MS m/z: 533 [M+H]⁺.

Step 4: 6-(2-(2-methyl-6-(trifluoromethyl)pyrimidin-4-yl)-2,6-diazaspiro[3.4]octan-6-yl)-5-propyl-1,5-dihydro-4H-pyrazolo[3,4-d]pyrimidin-4-one: Followed the general procedure M using 6-(2-(2-methyl-6-(trifluoromethyl)pyrimidin-4-yl)-2,6-diazaspiro[3.4]octan-6-yl)-5-propyl-1-(tetrahydro-2H-pyran-2-yl)-1,5-dihydro-4H-pyrazolo[3,4-d]pyrimidin-4-one (90 mg, 0.169 mmol, 1.0 equiv.) as the starting material to give 6-(2-(2-methyl-6-(trifluoromethyl)pyrimidin-4-yl)-2,6-diazaspiro[3.4]octan-6-yl)-5-propyl-1,5-dihydro-4H-pyrazolo[3,4-d]pyrimidin-4-one hydrochloride (80 mg) as a light brown oil. MS m/z: 449 [M+H]⁺.

Step 5: 1-(2,2-difluoroethyl)-6-(2-(2-methyl-6-(trifluoromethyl)pyrimidin-4-yl)-2,6-diazaspiro[3.4]octan-6-yl)-5-propyl-1,5-dihydro-4H-pyrazolo[3,4-d]pyrimidin-4-one: Followed the general procedure K using 6-(2-(2-methyl-6-(trifluoromethyl)pyrimidin-4-yl)-2,6-diazaspiro[3.4]octan-6-yl)-5-propyl-1,5-dihydro-4H-pyrazolo[3,4-d]pyrimidin-4-one hydrochloride (80 mg, 0.178 mmol, 1.0 equiv.) and 2,2-difluoroethyl trifluoromethanesulfonate (46 mg, 0.214 mmol, 1.2 equiv.) as the starting materials to give 1-(2,2-difluoroethyl)-6-(2-(2-methyl-6-(trifluoromethyl)pyrimidin-4-yl)-2,6-diazaspiro[3.4]octan-6-yl)-5-propyl-1,5-dihydro-4H-pyrazolo[3,4-d]pyrimidin-4-one (26.5 mg, 28%) as a white solid. ¹H NMR (400 MHz, DMSO-d₆) δ 7.94 (d, J=1.2 Hz, 1H), 6.64 (s, 1H), 6.43 (s, 1H), 4.65-4.59 (m, 2H), 4.45 (t, J=6.7 Hz, 2H), 4.15 (s, 2H), 4.11 (s, 2H), 3.80 (s, 2H), 3.64 (s, 2H), 2.42 (s, 3H), 2.23 (d, J=6.9 Hz, 2H), 1.80 (d, J=7.1 Hz, 2H), 0.98 (t, J=7.4 Hz, 3H). MS m/z: 513.15 [M+H]⁺.

### Example 1112: 1-(2,2-difluoroethyl)-5-isobutyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one

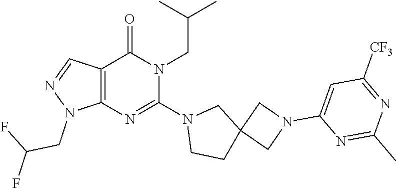 [SMILES: Cc1nc(N2CC3(CCN(c4nc5c(cnn5CC(F)F)c(=O)n4CC(C)C)C3)C2)cc(C(F)(F)F)n1]

Step 1: 5-isobutyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure I using 6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (75 mg, 0.153 mmol, 1.0 equiv.) and 1-iodo-2-methylpropane (33 mg, 0.184 mmol, 1.2 equiv.) as the starting materials to give 5-isobutyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (60 mg, 71%) as an off-white solid. MS m/z: 547 [M+H]⁺.

Step 2: 5-isobutyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure M using 5-isobutyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (60 mg, 0.169 mmol, 1.0 equiv.) as the starting material to give 5-isobutyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (50 mg, 92%) as a light brown oil. MS m/z: 463 [M+H]⁺.

Step 3: 1-(2,2-difluoroethyl)-5-isobutyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure K using 5-isobutyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one hydrochloride (50 mg, 0.108 mmol, 1.0 equiv.) and 2,2-difluoroethyl trifluoromethanesulfonate (27 mg, 0.130 mmol, 1.2 equiv.) as the starting materials to give 1-(2,2-difluoroethyl)-5-isobutyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (11.6 mg, 20%) as a white solid. ¹H NMR (400 MHz, DMSO-d₆) δ 7.95 (s, 1H), 6.64 (s, 1H), 6.56-6.27 (m, 1H), 4.71-4.51 (m, 2H), 4.28 (d, J=6.6 Hz, 2H), 4.16 (d, J=9.4 Hz, 2H), 4.09 (d, J=9.2 Hz, 2H), 3.82 (d, J=14.2 Hz, 2H), 3.64 (s, 2H), 2.43 (s, 3H), 2.24 (t, J=6.9 Hz, 2H), 2.11 (d, J=6.8 Hz, 1H), 0.99 (d, J=6.6 Hz, 6H). MS m/z: 527.35 [M+H]⁺.

### Example 1113: 5-(cyclopropylmethyl)-1-(2,2-difluoroethyl)-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one

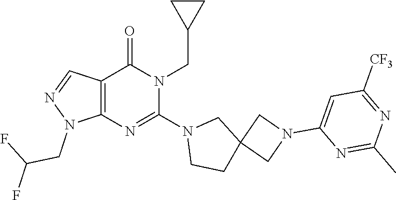 [SMILES: Cc1nc(N2CC3(CCN(c4nc5c(cnn5CC(F)F)c(=O)n4CC4CC4)C3)C2)cc(C(F)(F)F)n1]

Step 1: 6-chloro-5-(cyclopropylmethyl)-1-(tetrahydro-2H-pyran-2-yl)-1,5-dihydro-4H-pyrazolo[3,4-d]pyrimidin-4-one: Followed the general procedure K using 6-chloro-1-(tetrahydro-2H-pyran-2-yl)-1,5-dihydro-4H-pyrazolo[3,4-d]pyrimidin-4-one (500 mg, 2.0 mmol, 1.0 equiv.) and (iodomethyl)cyclopropane (430 mg, 2.4 mmol, 1.2 equiv.) as the starting materials to give 6-chloro-5-(cyclopropylmethyl)-1-(tetrahydro-2H-pyran-2-yl)-1,5-dihydro-4H-pyrazolo[3,4-d]pyrimidin-4-one (240 mg, 40%) as a colorless oil. MS m/z: 309[M+H]⁺.

Step 2: 5-(cyclopropylmethyl)-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure I using 6-chloro-5-(cyclopropylmethyl)-1-(tetrahydro-2H-pyran-2-yl)-1,5-dihydro-4H-pyrazolo[3,4-d]pyrimidin-4-one (240 mg, 0.78 mmol, 1.0 equiv.) and 2-(2-methyl-6-(trifluoromethyl)pyrimidin-4-yl)-2,6-diazaspiro[3.4]octane (212 mg, 0.78 mmol, 1.0 equiv.) as the starting materials to give 5-(cyclopropylmethyl)-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (145 mg, 34%) as a white solid. MS m/z: 545[M+H]⁺.

Step 3: 5-(cyclopropylmethyl)-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure H using 5-(cyclopropylmethyl)-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (145 mg) as the starting material to give the crude product 5-(cyclopropylmethyl)-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (100 mg). MS m/z: 461[M+H]⁺.

Step 4: 5-(cyclopropylmethyl)-1-(2,2-difluoroethyl)-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure K using 5-(cyclopropylmethyl)-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (100 mg, 0.217 mmol, 1.0 equiv.) and 2,2-difluoroethyl trifluoromethanesulfonate (70 mg, 0.326 mmol, 1.5 equiv.) as the starting materials to give 5-(cyclopropylmethyl)-1-(2,2-difluoroethyl)-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (39.3 mg, 40%) as a white solid. ¹H NMR (400 MHz, DMSO-d₆) δ 7.96 (s, 1H), 6.65 (s, 1H), 6.61-6.30 (m, 1H), 4.65-4.52 (m, 2H), 4.18-4.07 (m, 4H), 4.02 (d, J=6.8 Hz, 2H), 3.81 (s, 2H), 3.67-3.64 (m, 2H), 2.42 (s, 3H), 2.21 (t, J=6.4 Hz, 2H), 1.22-1.11 (m, 1H), 0.41 (d, J=8.0 Hz, 2H), 0.25 (d, J=5.2 Hz, 2H). MS m/z: 525.05 [M+H]⁺.

### Example 1114: 5-ethyl-6-(2-(2-methyl-6-(trifluoromethyl)pyrimidin-4-yl)-2,6-diazaspiro[3.4]octan-6-yl)-1-(2,2,2-trifluoroethyl)-1,5-dihydro-4H-pyrazolo[3,4-d]pyrimidin-4-one

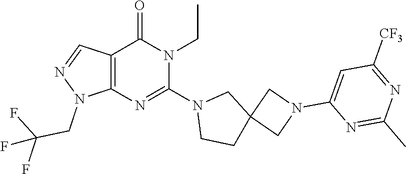 [SMILES: CCn1c(N2CCC3(CN(c4cc(C(F)(F)F)nc(C)n4)C3)C2)nc2c(cnn2CC(F)(F)F)c1=O]

Step 1: 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure I using 2-(2-methyl-6-(trifluoromethyl)pyrimidin-4-yl)-2,6-diazaspiro[3.4]octane (400 mg, 1.47 mmol, 1.0 equiv.) and 6-chloro-5-ethyl-1-(tetrahydro-2H-pyran-2-yl)-1,5-dihydro-4H-pyrazolo[3,4-d]pyrimidin-4-one (498 mg, 1.76 mmol, 1.2 equiv.) as the starting materials to give 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (515 mg, 67%) as a white solid. MS m/z: 519 [M+H]⁺.

Step 2: 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure M using 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (515 mg, 0.993 mmol, 1.0 equiv.) as the starting material to give 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (300 mg, 69%) as a white solid. MS m/z: 435 [M+H]⁺.

Step 3: 5-ethyl-6-(2-(2-methyl-6-(trifluoromethyl)pyrimidin-4-yl)-2,6-diazaspiro[3.4]octan-6-yl)-1-(2,2,2-trifluoroethyl)-1,5-dihydro-4H-pyrazolo[3,4-d]pyrimidin-4-one: Followed the general procedure K using 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (150 mg, 0.345 mmol, 1.0 equiv.) and 2,2-difluoroethyl trifluoromethanesulfonate (96.4 mg, 0.414 mmol, 1.2 equiv.) as the starting materials to give 5-ethyl-6-(2-(2-methyl-6-(trifluoromethyl)pyrimidin-4-yl)-2,6-diazaspiro[3.4]octan-6-yl)-1-(2,2,2-trifluoroethyl)-1,5-dihydro-4H-pyrazolo[3,4-d]pyrimidin-4-one (37.8 mg, 21%) as a white solid. ¹H NMR (400 MHz, DMSO-d₆) δ 7.99 (s, 1H), 6.64 (s, 1H), 5.08-4.97 (m, 2H), 4.16 (d, J=9.4 Hz, 2H), 4.09 (d, J=9.3 Hz, 2H), 4.06-4.01 (m, 2H), 3.82 (d, J=5.2 Hz, 2H), 3.66 (s, 2H), 2.42 (s, 3H), 2.21 (t, J=6.9 Hz, 2H), 1.26 (t, J=6.9 Hz, 3H). MS m/z: 517.1 [M+H]⁺.

### Example 1115: 5-ethyl-6-(2-(2-methyl-6-(trifluoromethyl)pyrimidin-4-yl)-2,6-diazaspiro[3.4]octan-6-yl)-1-(oxetan-3-yl)-1,5-dihydro-4H-pyrazolo[3,4-d]pyrimidin-4-one

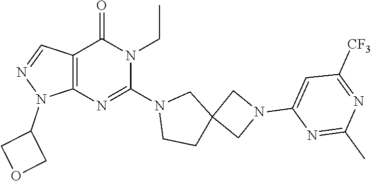 [SMILES: CCn1c(N2CCC3(CN(c4cc(C(F)(F)F)nc(C)n4)C3)C2)nc2c(cnn2C2COC2)c1=O]

Step 1: 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one: Followed the general procedure I using 2-(2-methyl-6-(trifluoromethyl)pyrimidin-4-yl)-2,6-diazaspiro[3.4]octane (210 mg, 0.77 mmol, 1.0 equiv.) and 6-chloro-5-ethyl-1-(tetrahydro-2H-pyran-2-yl)-1,5-dihydro-4H-pyrazolo[3,4-d]pyrimidin-4-one (261 mg, 0.926 mmol, 1.2 equiv.) as the starting materials to give 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (255 mg, 64%) as a colorless oil. MS m/z: 519[M+H]⁺.

Step 2: 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one: Followed the general procedure M using 5-ethyl-6-(2-(2-methyl-6-(trifluoromethyl)pyrimidin-4-yl)-2,6-diazaspiro[3.4]octan-6-yl)-1-(tetrahydro-2H-pyran-2-yl)-1,5-dihydro-4H-pyrazolo[3,4-d]pyrimidin-4-one (255 mg) as the starting material to give the crude product 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (150 mg) MS m/z: 435 [M+H]⁺.

Step 3: 5-ethyl-6-(2-(2-methyl-6-(trifluoromethyl)pyrimidin-4-yl)-2,6-diazaspiro[3.4]octan-6-yl)-1-(oxetan-3-yl)-1,5-dihydro-4H-pyrazolo[3,4-d]pyrimidin-4-one: Followed the general procedure K using 5-ethyl-6-(2-(2-methyl-6-(trifluoromethyl)pyrimidin-4-yl)-2,6-diazaspiro[3.4]octan-6-yl)-1,5-dihydro-4H-pyrazolo[3,4-d]pyrimidin-4-one (150 mg, 0.346 mmol, 1.0 equiv.) and oxetan-3-ylmethyl trifluoromethanesulfonate (114 mg, 0.518 mmol, 1.5 equiv.) as the starting materials to give 5-ethyl-6-(2-(2-methyl-6-(trifluoromethyl)pyrimidin-4-yl)-2,6-diazaspiro[3.4]octan-6-yl)-1-(oxetan-3-yl)-1,5-dihydro-4H-pyrazolo[3,4-d]pyrimidin-4-one (55.6 mg, 33%) as a white solid. ¹H NMR (400 MHz, DMSO-d₆) δ 8.00 (s, 1H), 6.65 (s, 1H), 5.82-5.69 (m, 1H), 5.03-4.89 (m, 4H), 4.19-3.99 (m, 6H), 3.79 (s, 2H), 3.63 (t, J=6.8 Hz, 2H), 2.42 (s, 3H), 2.20 (t, J=7.0 Hz, 2H), 1.23 (t, J=6.8 Hz, 3H). MS m/z: 491.05 [M+H]⁺.

### Example 1116: 1-(2,2-difluoropropyl)-6-(2-(2-(trifluoromethyl)pyridin-4-yl)-2,6-diazaspiro[3.4]octan-6-yl)-1H-pyrazolo[3,4-b]pyrazine

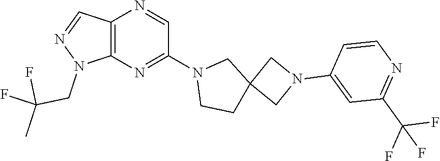 [SMILES: CC(F)(F)Cn1ncc2ncc(N3CCC4(CN(c5ccnc(C(F)(F)F)c5)C4)C3)nc21]

Step 1: 1-(6-chloro-1H-pyrazolo[3,4-b]pyrazin-1-yl)propan-2-one. A solution of 6-chloro-1H-pyrazolo[3,4-b]pyrazine (250 mg, 1.62 mmol, 1 equiv.), 1-bromopropan-2-one (332 mg, 2.42 mmol, 1.5 equiv.) and Cs₂CO₃ (1.05 g, 3.23 mmol, 2 equiv.) in DMF (3 mL) were stirred for 3 h at 60° C. Desired product could be detected by LCMS. The residue was purified by silica gel column chromatography, eluted with EtOAc/PE=2/1, to afford 1-(6-chloro-1H-pyrazolo[3,4-b]pyrazin-1-yl)propan-2-one (140 mg, 41%) as a yellow solid. MS m/z: 210 [M+H]⁺

Step 2: 1-(6-(2-(2-(trifluoromethyl)pyridin-4-yl)-2,6-diazaspiro[3.4]octan-6-yl)-1H-pyrazolo[3,4-b]pyrazin-1-yl)propan-2-one: Followed the general procedure I using 1-(6-chloro-1H-pyrazolo[3,4-b]pyrazin-1-yl)propan-2-one (70 mg, 0.332 mmol, 1 equiv.) and 2-(2-(trifluoromethyl)pyridin-4-yl)-2,6-diazaspiro[3.4]octane (94.0 mg, 0.365 mmol, 1.1 equiv.) as the starting materials to give 1-(6-(2-(2-(trifluoromethyl)pyridin-4-yl)-2,6-diazaspiro[3.4]octan-6-yl)-1H-pyrazolo[3,4-b]pyrazin-1-yl)propan-2-one (140 mg, 98%) as a yellow solid. MS m/z: 431[M+H]⁺.

Step 3: 1-(2,2-difluoropropyl)-6-(2-(2-(trifluoromethyl)pyridin-4-yl)-2,6-diazaspiro[3.4]octan-6-yl)-1H-pyrazolo[3,4-b]pyrazine: Followed the general procedure AD using 1-(6-(2-(2-(trifluoromethyl)pyridin-4-yl)-2,6-diazaspiro[3.4]octan-6-yl)-1H-pyrazolo[3,4-b]pyrazin-1-yl)propan-2-one (100 mg, 0.232 mmol, 1 equiv.) as the starting material to give 1-(2,2-difluoropropyl)-6-(2-(2-(trifluoromethyl)pyridin-4-yl)-2,6-diazaspiro[3.4]octan-6-yl)-1H-pyrazolo[3,4-b]pyrazine (26.5 mg, 24%) as a white solid. ¹H NMR (400 MHz, DMSO-d₆) δ 8.25 (d, J=5.6 Hz, 1H), 8.21-8.05 (m, 2H), 6.76 (d, J=2.3 Hz, 1H), 6.58 (dd, J=5.7, 2.3 Hz, 1H), 4.69 (t, J=12.9 Hz, 2H), 4.17-3.97 (m, 4H), 3.86 (s, 2H), 3.67 (t, J=6.9 Hz, 2H), 2.30 (d, J=7.0 Hz, 2H), 1.65 (t, J=19.1 Hz, 3H). MS m/z: 454.20[M+H]⁺.

### Example 1117: 1-(2,2-difluoropropyl)-6-(2-(2-methyl-6-(trifluoromethyl)pyrimidin-4-yl)-2,6-diazaspiro[3.4]octan-6-yl)-1H-pyrazolo[3,4-b]pyrazine

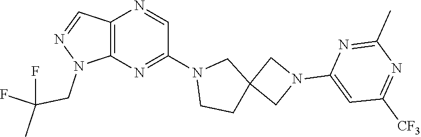 [SMILES: Cc1nc(N2CC3(CCN(c4cnc5cnn(CC(C)(F)F)c5n4)C3)C2)cc(C(F)(F)F)n1]

Step 1: 1-(6-(2-(2-methyl-6-(trifluoromethyl)pyrimidin-4-yl)-2,6-diazaspiro[3.4]octan-6-yl)-1H-pyrazolo[3,4-b]pyrazin-1-yl)propan-2-one: Followed the general procedure I using 1-(6-chloro-1H-pyrazolo[3,4-b]pyrazin-1-yl)propan-2-one (50 mg, 0.237 mmol, 1.0 equiv.) and 2-(2-methyl-6-(trifluoromethyl)pyrimidin-4-yl)-2,6-diazaspiro[3.4]octane (77.5 mg, 0.284 mmol, 1.2 equiv.) as the starting materials to give 1-(6-(2-(2-methyl-6-(trifluoromethyl)pyrimidin-4-yl)-2,6-diazaspiro[3.4]octan-6-yl)-1H-pyrazolo[3,4-b]pyrazin-1-yl)propan-2-one (50 mg, 47%) as a white solid. MS m/z: 447 [M+H]⁺.

Step 2: 1-(2,2-difluoropropyl)-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure AD using 1-(6-(2-(2-methyl-6-(trifluoromethyl)pyrimidin-4-yl)-2,6-diazaspiro[3.4]octan-6-yl)-1H-pyrazolo[3,4-b]pyrazin-1-yl)propan-2-one (50 mg, 0.112 mmol, 1.0 equiv.) as the starting material to give 1-(2,2-difluoropropyl)-6-(2-(2-methyl-6-(trifluoromethyl)pyrimidin-4-yl)-2,6-diazaspiro[3.4]octan-6-yl)-1H-pyrazolo[3,4-b]pyrazine (22 mg, 42%) as a white solid. ¹H NMR (400 MHz, DMSO-d₆) δ 8.11 (d, J=17.7 Hz, 2H), 6.66 (s, 1H), 4.69 (t, J=12.8 Hz, 2H), 4.18 (d, J=9.4 Hz, 2H), 4.12 (d, J=9.3 Hz, 2H), 3.85 (s, 2H), 3.67 (t, J=6.9 Hz, 2H), 2.43 (s, 3H), 2.31 (d, J=6.2 Hz, 2H), 1.65 (t, J=19.2 Hz, 3H). MS m/z: 469.15 [M+H]⁺.

### Example 1118: 6-(6-(1-(2,2-difluoroethyl)-1H-pyrazol-3-yl)pyrazin-2-yl)-2-(2-methyl-6-(trifluoromethyl)pyrimidin-4-yl)-2,6-diazaspiro[3.4]octane

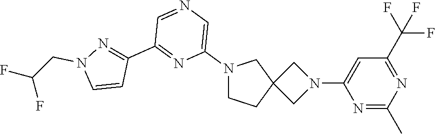 [SMILES: Cc1nc(N2CC3(CCN(c4cncc(-c5ccn(CC(F)F)n5)n4)C3)C2)cc(C(F)(F)F)n1]

Step 1: 6-(6-(1-(2,2-difluoroethyl)-1H-pyrazol-3-yl)pyrazin-2-yl)-2-(2-methyl-6-(trifluoromethyl)pyrimidin-4-yl)-2,6-diazaspiro[3.4]octane: Followed the general procedure K using 6-(6-(1H-pyrazol-5-yl)pyrazin-2-yl)-2-(2-methyl-6-(trifluoromethyl)pyrimidin-4-yl)-2,6-diazaspiro[3.4]octane (100 mg, 0.24 mmol, 1.0 equiv.) and 2,2-difluoroethyl trifluoromethanesulfonate (77 mg, 0.36 mmol, 1.5 equiv.) as the starting materials to give 6-(6-(1-(2,2-difluoroethyl)-1H-pyrazol-3-yl)pyrazin-2-yl)-2-(2-methyl-6-(trifluoromethyl)pyrimidin-4-yl)-2,6-diazaspiro[3.4]octane (28.7 mg, 25%) as a white solid. ¹H NMR (400 MHz, DMSO-d₆) δ 8.31 (s, 1H), 7.92-7.82 (m, 2H), 6.86 (d, J=2.4 Hz, 1H), 6.66 (s, 1H), 6.60-6.23 (m, 1H), 4.80-4.64 (m, 2H), 4.21-4.08 (m, 4H), 3.78 (s, 2H), 3.60-3.57 (m, 2H), 2.43 (s, 3H), 2.32-2.24 (m, 2H). MS m/z: 481.15 [M+H]⁺.

### Example 1119: 6-(6-(1-(2,2-difluoroethyl)-1H-pyrazol-5-yl)pyrazin-2-yl)-2-(2-methyl-6-(trifluoromethyl)pyrimidin-4-yl)-2,6-diazaspiro[3.4]octane

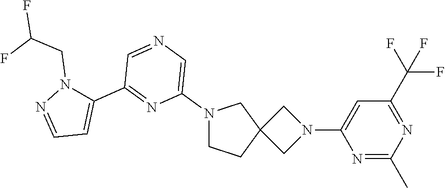 [SMILES: Cc1nc(N2CC3(CCN(c4cncc(-c5ccnn5CC(F)F)n4)C3)C2)cc(C(F)(F)F)n1]

Step 1: 6-(6-(1-(2,2-difluoroethyl)-1H-pyrazol-5-yl)pyrazin-2-yl)-2-(2-methyl-6-(trifluoromethyl)pyrimidin-4-yl)-2,6-diazaspiro[3.4]octane: Followed the general procedure K using 6-(6-(1H-pyrazol-5-yl)pyrazin-2-yl)-2-(2-methyl-6-(trifluoromethyl)pyrimidin-4-yl)-2,6-diazaspiro[3.4]octane (100 mg, 0.24 mmol, 1.0 equiv.) and 2,2-difluoroethyl trifluoromethanesulfonate (77 mg, 0.36 mmol, 1.5 equiv.) as the starting materials, to give 6-(6-(1-(2,2-difluoroethyl)-1H-pyrazol-5-yl)pyrazin-2-yl)-2-(2-methyl-6-(trifluoromethyl)pyrimidin-4-yl)-2,6-diazaspiro[3.4]octane (44.7 mg, 39%) as a white solid. ¹H NMR (400 MHz, DMSO-d₆) δ 8.28 (s, 1H), 7.96 (s, 1H), 7.67-7.61 (m, 1H), 7.00 (d, J=2.0 Hz, 1H), 6.67 (s, 1H), 6.59-6.26 (m, 1H), 5.24-5.09 (m, 2H), 4.17-4.12 (m, 4H), 3.77 (s, 2H), 3.59-3.58 (m, 2H), 2.43 (s, 3H), 2.35-2.28 (m, 2H). MS m/z: 481.15 [M+H]⁺.

### Example 1120: 2-(2-methyl-6-(trifluoromethyl)pyrimidin-4-yl)-6-(6-(1-(2,2,2-trifluoroethyl)-1H-pyrazol-3-yl)pyrazin-2-yl)-2,6-diazaspiro[3.4]octane; Example 1121: 2-(2-methyl-6-(trifluoromethyl)pyrimidin-4-yl)-6-(6-(1-(2,2,2-trifluoroethyl)-1H-pyrazol-5-yl)pyrazin-2-yl)-2,6-diazaspiro[3.4]octane

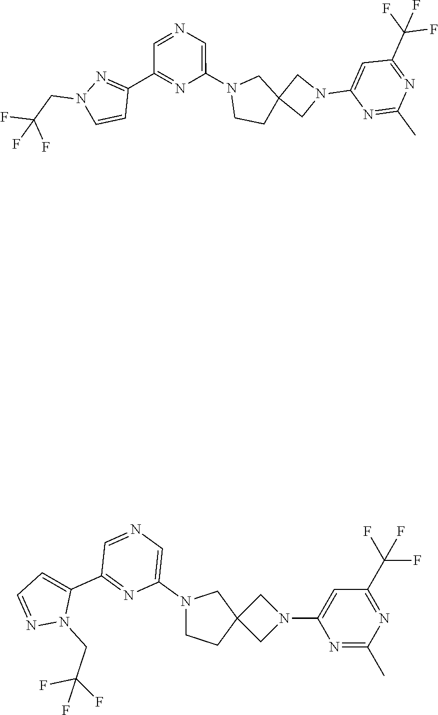 [SMILES: Cc1nc(N2CC3(CCN(c4cncc(-c5ccn(CC(F)(F)F)n5)n4)C3)C2)cc(C(F)(F)F)n1 ; Cc1nc(N2CC3(CCN(c4cncc(-c5ccnn5CC(F)(F)F)n4)C3)C2)cc(C(F)(F)F)n1]

Step 1: 6-(6-bromopyrazin-2-yl)-2-(2-methyl-6-(trifluoromethyl)pyrimidin-4-yl)-2,6-diazaspiro[3.4]octane: Followed the general procedure I using 2-(2-methyl-6-(trifluoromethyl)pyrimidin-4-yl)-2,6-diazaspiro[3.4]octane (110 mg, 0.404 mmol, 1.0 equiv.) and 2,6-dibromopyrazine (115 mg, 0.49 mmol, 1.2 equiv.) as the starting materials to give 6-(6-bromopyrazin-2-yl)-2-(2-methyl-6-(trifluoromethyl)pyrimidin-4-yl)-2,6-diazaspiro[3.4]octane (130 mg, 75%) as a colorless oil. MS m/z: 429[M+H]⁺.

Step 2: 2-(2-methyl-6-(trifluoromethyl)pyrimidin-4-yl)-6-(6-(1-(tetrahydro-2H-pyran-2-yl)-1H-pyrazol-5-yl)pyrazin-2-yl)-2,6-diazaspiro[3.4]octane: Followed the general procedure L using 6-(6-bromopyrazin-2-yl)-2-(2-methyl-6-(trifluoromethyl)pyrimidin-4-yl)-2,6-diazaspiro[3.4]octane (110 mg, 0.256 mmol, 1.0 equiv.) and 1-(tetrahydro-2H-pyran-2-yl)-5-(4,4,5,5-tetramethyl-1,3,2-dioxaborolan-2-yl)-1H-pyrazole (86 mg, 0.308 mmol, 1.2 equiv.) as the starting materials to give 2-(2-methyl-6-(trifluoromethyl)pyrimidin-4-yl)-6-(6-(1-(tetrahydro-2H-pyran-2-yl)-1H-pyrazol-5-yl)pyrazin-2-yl)-2,6-diazaspiro[3.4]octane (120 mg, 93%) as a white solid. MS m/z: 501[M+H]⁺.

Step 3: 6-(6-(1H-pyrazol-5-yl)pyrazin-2-yl)-2-(2-methyl-6-(trifluoromethyl)pyrimidin-4-yl)-2,6-diazaspiro[3.4]octane: Followed the general procedure H using 2-(2-methyl-6-(trifluoromethyl)pyrimidin-4-yl)-6-(6-(1-(tetrahydro-2H-pyran-2-yl)-1H-pyrazol-5-yl)pyrazin-2-yl)-2,6-diazaspiro[3.4]octane (120 mg, 0.24 mmol, 1.0 equiv.) as the starting material to give 6-(6-(1H-pyrazol-5-yl)pyrazin-2-yl)-2-(2-methyl-6-(trifluoromethyl)pyrimidin-4-yl)-2,6-diazaspiro[3.4]octane (95 mg, 95%) as a white solid. MS m/z: 417 [M+H]⁺.

Step 4: 2-(2-methyl-6-(trifluoromethyl)pyrimidin-4-yl)-6-(6-(1-(2,2,2-trifluoroethyl)-1H-pyrazol-3-yl)pyrazin-2-yl)-2,6-diazaspiro[3.4]octane: Followed the general procedure K using 6-(6-(1H-pyrazol-5-yl)pyrazin-2-yl)-2-(2-methyl-6-(trifluoromethyl)pyrimidin-4-yl)-2,6-diazaspiro[3.4]octane (95 mg, 0.228 mmol, 1.0 equiv.) and 2,2,2-trifluoroethyl trifluoromethanesulfonate (79 mg, 0.342 mmol, 1.5 equiv.) as the starting materials to give the crude product. The crude product was purified by Prep-HPLC with the following conditions (Column: Xselect CSH C18 OBD Column 30*150 mm 5 m, n; Mobile Phase A: Water (0.05% TFA), Mobile Phase B: ACN; Flow rate: 60 mL/min; Gradient: 36% B to 47% B in 8 min, 47% B; Wave Length: 254/220 nm; RT1(min): 9.95/10.78; Number Of Runs: 0) to afford 2-(2-methyl-6-(trifluoromethyl)pyrimidin-4-yl)-6-(6-(1-(2,2,2-trifluoroethyl)-1H-pyrazol-3-yl)pyrazin-2-yl)-2,6-diazaspiro[3.4]octane (45.5 mg, 40%) as a white solid and 2-(2-methyl-6-(trifluoromethyl)pyrimidin-4-yl)-6-(6-(1-(2,2,2-trifluoroethyl)-1H-pyrazol-5-yl)pyrazin-2-yl)-2,6-diazaspiro[3.4]octane (15.6 mg, 14%) as a white solid.

2-(2-methyl-6-(trifluoromethyl)pyrimidin-4-yl)-6-(6-(1-(2,2,2-trifluoroethyl)-1H-pyrazol-3-yl)pyrazin-2-yl)-2,6-diazaspiro[3.4]octane ¹H NMR (400 MHz, DMSO-d₆) δ 8.31 (s, 1H), 7.95 (d, J=2.4 Hz, 1H), 7.90 (s, 1H), 6.91 (d, J=2.4 Hz, 1H), 6.66 (s, 1H), 5.31-5.19 (m, 2H), 4.21-4.07 (m, 4H), 3.78 (s, 2H), 3.61-3.56 (m, 2H), 2.43 (s, 3H), 2.31-2.24 (m, 2H). MS m/z: 499.1 [M+H]⁺.

2-(2-methyl-6-(trifluoromethyl)pyrimidin-4-yl)-6-(6-(1-(2,2,2-trifluoroethyl)-1H-pyrazol-5-yl)pyrazin-2-yl)-2,6-diazaspiro[3.4]octane ¹H NMR (400 MHz, DMSO-d₆) δ 8.33 (s, 1H), 7.98 (s, 1H), 7.70 (d, J=2.0 Hz, 1H), 7.07 (d, J=2.0 Hz, 1H), 6.68 (s, 1H), 5.82-5.69 (m, 2H), 4.19-4.09 (m, 4H), 3.77 (s, 2H), 3.61-3.56 (m, 2H) 2.43 (s, 3H), 2.31 (t, J=6.8 Hz, 2H). MS m/z: 499.1 [M+H]⁺.

### Example 1122: (S)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine, assumed; Example 1123: (R)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine, assumed

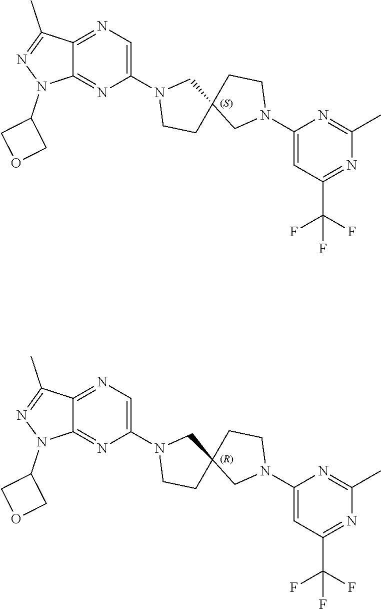

Step 1: (S)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine; (R)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine. 3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine was purified by prep. HPLC with the following conditions: Column: CHIRAL ART Cellulose-SZ, 4.6*50 mm, 3 m; Mobile Phase A: Hex (0.1% DEA): EtOH=70:30; Flow rate: 1 mL/min; Gradient: 0% B to 0% B; Injection Volume: Sul mL to afford (S)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine, assumed (24 mg, 26%) and (R)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine, assumed (22.5 mg, 25%) as solids.

(S)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine, assumed ¹H NMR (400 MHz, Chloroform-d) δ 7.87 (s, 1H), 6.60-6.40 (m, 1H), 5.95-5.78 (m, 1H), 5.37-5.24 (m, 2H), 5.11-4.93 (m, 2H), 3.95-3.48 (m, 8H), 2.68-2.50 (m, 6H), 2.24-2.03 (m, 4H). MS m/z: 475.35 [M+H]⁺.

(R)-3-methyl-6-(7-(2-methyl-6-(trifluoromethyl)pyrimidin-4-yl)-2,7-diazaspiro[4.4]nonan-2-yl)-1-(oxetan-3-yl)-1H-pyrazolo[3,4-b]pyrazine LCMS (ES, m/z): 475.35 [M+H]⁺. ¹H NMR (400 MHz, Chloroform-d) δ 7.87 (s, 1H), 6.60-6.40 (m, 1H), 5.95-5.78 (m, 1H), 5.37-5.24 (m, 2H), 5.11-4.93 (m, 2H), 3.95-3.48 (m, 8H), 2.68-2.50 (m, 6H), 2.24-2.03 (m, 4H). MS m/z: 475.35 [M+H]⁺.

### Example 1124: 6-(6-(1-(2,2-difluoroethyl)-1H-pyrazol-4-yl)pyrazin-2-yl)-2-(2-methyl-6-(trifluoromethyl)pyrimidin-4-yl)-2,6-diazaspiro[3.4]octane

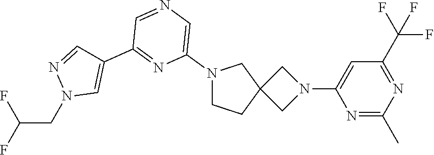 [SMILES: Cc1nc(N2CC3(CCN(c4cncc(-c5cnn(CC(F)F)c5)n4)C3)C2)cc(C(F)(F)F)n1]

Step 1: 6-(6-(1-(2,2-difluoroethyl)-1H-pyrazol-4-yl)pyrazin-2-yl)-2-(2-methyl-6-(trifluoromethyl)pyrimidin-4-yl)-2,6-diazaspiro[3.4]octane: Followed the general procedure L using 6-(6-bromopyrazin-2-yl)-2-(2-methyl-6-(trifluoromethyl)pyrimidin-4-yl)-2,6-diazaspiro[3.4]octane (110 mg, 0.267 mmol, 1.0 equiv.) and 1-(2,2-difluoroethyl)-4-(4,4,5,5-tetramethyl-1,3,2-dioxaborolan-2-yl)-1H-pyrazole (79.5 mg, 0.308 mmol, 1.2 equiv.) as the starting materials to give 6-(6-(1-(2,2-difluoroethyl)-1H-pyrazol-4-yl)pyrazin-2-yl)-2-(2-methyl-6-(trifluoromethyl)pyrimidin-4-yl)-2,6-diazaspiro[3.4]octane (75.2 mg, 77%) as a white solid. ¹H NMR (400 MHz, DMSO-d₆) δ 8.35 (s, 1H), 8.15 (s, 1H), 8.11 (s, 1H), 7.78 (s, 1H), 6.66 (s, 1H), 6.58-6.18 (m, 1H), 4.75-4.61 (m, 2H), 4.21-4.07 (m, 4H), 3.76 (s, 2H), 3.57 (t, J=6.8 Hz, 2H), 2.43 (s, 3H), 2.28 (t, J=6.8 Hz, 2H). MS m/z: 481.15 [M+H]⁺.

### Example 1125: 1-neopentyl-6-(2-(2-(trifluoromethyl)pyridin-4-yl)-2,6-diazaspiro[3.4]octan-6-yl)-1H-pyrazolo[3,4-b]pyrazine

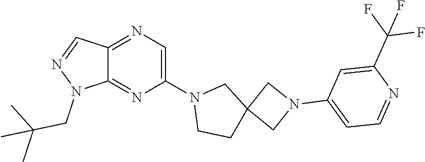 [SMILES: CC(C)(C)Cn1ncc2ncc(N3CCC4(CN(c5ccnc(C(F)(F)F)c5)C4)C3)nc21]

Step 1: octane-1-sulfonohydrazide. A solution of octane-1-sulfonyl chloride (2 g, 9.40 mmol, 1.0 equiv.) and hydrazine (0.32 g, 9.87 mmol, 1.05 equiv.) in THF (10 mL) was stirred for 4 h at 0° C. under argon atmosphere. The resulting mixture was concentrated under vacuum. The resulting mixture was extracted with EtOAc (40 mL). The combined organic layers were washed with water (2×20 mL), dried over anhydrous Na₂SO₄. After filtration, the filtrate was concentrated under reduced pressure. The crude product (1.8 g) was used in the next step directly without further purification. MS m/z: 209 [M+H]⁺.

Step 2: N′-methyleneoctane-1-sulfonohydrazide. A solution of octane-1-sulfonohydrazide (400 mg, 1.92 mmol, 1.0 equiv.) and formaldehyde solution (346 mg, 11.5 mmol, 6.0 equiv.) in THF (10 mL) was stirred for 3 h at room temperature. The resulting mixture was concentrated under vacuum. The crude product was used in the next step directly without further purification. MS m/z: 220 [M+H]⁺

Step 3: 1-neopentyl-6-(2-(2-(trifluoromethyl)pyridin-4-yl)-2,6-diazaspiro[3.4]octan-6-yl)-1H-pyrazolo[3,4-b]pyrazine. A solution of 1-(2-methylallyl)-6-(2-(2-(trifluoromethyl)pyridin-4-yl)-2,6-diazaspiro[3.4]octan-6-yl)-1H-pyrazolo[3,4-b]pyrazine (117 mg, 0.272 mmol, 1.0 equiv.), N′-methyleneoctane-1-sulfonohydrazide (300 mg, 1.36 mmol, 5.0 equiv.), Fe(acac)₃ (240 mg, 0.681 mmol, 2.5 equiv.) and phenylsilane (118 mg, 1.09 mmol, 5.0 equiv.) in MeOH (10 mL) was stirred for overnight at room temperature under argon atmosphere. The resulting mixture was concentrated under vacuum. The residue was dissolved in MeOH (10 mL). The resulting mixture was stirred for 2 h at 60° C. Then, The resulting mixture was concentrated under vacuum. The residue was purified by silica gel column chromatography, eluted with PE/EA (1:1) to afford crude product. The crude product (130 mg) was purified by Prep-HPLC to afford 1-neopentyl-6-(2-(2-(trifluoromethyl)pyridin-4-yl)-2,6-diazaspiro[3.4]octan-6-yl)-1H-pyrazolo[3,4-b]pyrazine (24 mg, 3%) as a white solid. ¹H NMR (400 MHz, DMSO-d₆) δ 8.25 (d, J=5.7 Hz, 1H), 8.04 (d, J=1.2 Hz, 2H), 6.76 (d, J=2.2 Hz, 1H), 6.58 (dd, J=5.7, 2.3 Hz, 1H), 4.14-3.99 (m, 6H), 3.84 (s, 2H), 3.65 (t, J=6.9 Hz, 2H), 2.31 (t, J=6.8 Hz, 2H), 0.94 (s, 9H). MS m/z: 446.3 [M+H]⁺.

### Example 1126: 1-isobutyl-6-(2-(2-(trifluoromethyl)pyridin-4-yl)-2,6-diazaspiro[3.4]octan-6-yl)-1H-pyrazolo[3,4-b]pyrazine

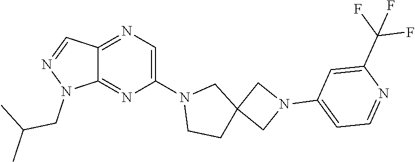 [SMILES: CC(C)Cn1ncc2ncc(N3CCC4(CN(c5ccnc(C(F)(F)F)c5)C4)C3)nc21]

Step 1: 6-chloro-1-(2-methylallyl)-1H-pyrazolo[3,4-b]pyrazine: Followed the general procedure I using 6-chloro-1H-pyrazolo[3,4-b]pyrazine (500 mg, 3.32 mmol, 1.0 equiv.) and 3-bromo-2-methylprop-1-ene (874 mg, 6.47 mmol, 2.0 equiv.) as the starting materials to give 6-chloro-1-(2-methylallyl)-1H-pyrazolo[3,4-b]pyrazine (500 mg, 74%) as a white solid. MS m/z: 209 [M+H]⁺.

Step 2: 1-(2-methylallyl)-6-(2-(2-(trifluoromethyl)pyridin-4-yl)-2,6-diazaspiro[3.4]octan-6-yl)-1H-pyrazolo[3,4-b]pyrazine: Followed the general procedure I using 6-chloro-1-(2-methylallyl)-1H-pyrazolo[3,4-b]pyrazine (300 mg, 1.43 mmol, 1.0 equiv.) and 2-(2-(trifluoromethyl)pyridin-4-yl)-2,6-diazaspiro[3.4]octane (370 mg, 1.43 mmol, 1.0 equiv.) as the starting materials to give 1-(2-methylallyl)-6-(2-(2-(trifluoromethyl)pyridin-4-yl)-2,6-diazaspiro[3.4]octan-6-yl)-1H-pyrazolo[3,4-b]pyrazine (450 mg, 73%) as a white solid. MS m/z: 429 [M+H]⁺.

Step 3: 1-isobutyl-6-(2-(2-(trifluoromethyl)pyridin-4-yl)-2,6-diazaspiro[3.4]octan-6-yl)-1H-pyrazolo[3,4-b]pyrazine: Followed the general procedure U using 1-(2-methylallyl)-6-(2-(2-(trifluoromethyl)pyridin-4-yl)-2,6-diazaspiro[3.4]octan-6-yl)-1H-pyrazolo[3,4-b]pyrazine (120 mg, 279 μmol, 1.0 equiv.) as the starting material to give 1-isobutyl-6-(2-(2-(trifluoromethyl)pyridin-4-yl)-2,6-diazaspiro[3.4]octan-6-yl)-1H-pyrazolo[3,4-b]pyrazine (60 mg, 50%) as a white solid. ¹H NMR (400 MHz, DMSO-d₆) δ 8.25 (d, J=5.6 Hz, 1H), 8.13-7.97 (m, 2H), 6.76 (d, J=2.2 Hz, 1H), 6.58 (dd, J=5.7, 2.3 Hz, 1H), 4.15-3.95 (m, 6H), 3.84 (s, 2H), 3.66 (t, J=6.9 Hz, 2H), 2.28 (dt, J=19.2, 6.8 Hz, 3H), 0.84 (d, J=6.7 Hz, 6H). MS m/z: 432.1 [M+H]⁺.

### Example 1127: 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-d]pyrimidine

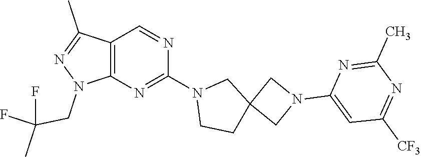 [SMILES: Cc1nc(N2CC3(CCN(c4ncc5c(C)nn(CC(C)(F)F)c5n4)C3)C2)cc(C(F)(F)F)n1]

Step 1: 1-(6-chloro-3-methyl-1H-pyrazolo[3,4-d]pyrimidin-1-yl)propan-2-one: Followed the general procedure K using 6-chloro-3-methyl-1H-pyrazolo[3,4-d]pyrimidine (200 mg, 1.18 mmol, 1.0 equiv.) and bromoacetone (195 mg, 1.42 mmol, 1.2 equiv.) as the starting materials to give 1-(6-chloro-3-methyl-1H-pyrazolo[3,4-d]pyrimidin-1-yl)propan-2-one (120 mg, 45%) as a yellow solid. MS m/z: 225 [M+H]⁺.

Step 2: 1-(3-methyl-6-(2-(2-methyl-6-(trifluoromethyl)pyrimidin-4-yl)-2,6-diazaspiro[3.4]octan-6-yl)-1H-pyrazolo[3,4-d]pyrimidin-1-yl)propan-2-one: Followed the general procedure I using 1-(6-chloro-3-methyl-1H-pyrazolo[3,4-d]pyrimidin-1-yl)propan-2-one (60 mg, 0.267 mmol, 1.0 equiv.) and 2-(2-methyl-6-(trifluoromethyl)pyrimidin-4-yl)-2,6-diazaspiro[3.4]octane (87 mg, 0.320 mmol, 1.2 equiv.) as the starting materials to give 1-(3-methyl-6-(2-(2-methyl-6-(trifluoromethyl)pyrimidin-4-yl)-2,6-diazaspiro[3.4]octan-6-yl)-1H-pyrazolo[3,4-d]pyrimidin-1-yl)propan-2-one (100 mg, 81%) as a yellow oil. MS m/z: 461 [M+H]⁺.

Step 3: 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-d]pyrimidine: Followed the general procedure AD using 1-(3-methyl-6-(2-(2-methyl-6-(trifluoromethyl)pyrimidin-4-yl)-2,6-diazaspiro[3.4]octan-6-yl)-1H-pyrazolo[3,4-d]pyrimidin-1-yl)propan-2-one (90 mg, 0.195 mmol, 1.0 equiv.) as the starting material to give 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-d]pyrimidine (21.2 mg, 22%) as a white solid. ¹H NMR (400 MHz, DMSO-d₆) δ 8.88 (s, 1H), 6.64 (s, 1H), 4.57 (t, J=12.7 Hz, 2H), 4.19-4.08 (m, 4H), 3.92-3.55 (m, 4H), 2.45-2.39 (m, 6H), 2.25 (t, J=6.9 Hz, 2H), 1.65 (t, J=19.1 Hz, 3H). MS m/z: 483.30 [M+H]⁺.

### Example 1128: 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-b]pyrazine

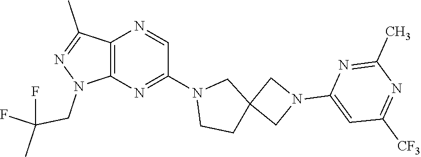 [SMILES: Cc1nc(N2CC3(CCN(c4cnc5c(C)nn(CC(C)(F)F)c5n4)C3)C2)cc(C(F)(F)F)n1]

Step 1: 1-(6-chloro-3-methyl-1H-pyrazolo[3,4-b]pyrazin-1-yl)propan-2-one: Followed the general procedure K using 6-chloro-3-methyl-1H-pyrazolo[3,4-b]pyrazine (100 mg, 0.593 mmol, 1.0 equiv.) and bromoacetone (97.5 mg, 0.712 mmol, 1.2 equiv.) as the starting materials to give 1-(6-chloro-3-methyl-1H-pyrazolo[3,4-b]pyrazin-1-yl)propan-2-one (100 mg, 75%) as a yellow solid. MS m/z: 225 [M+H]⁺.

Step 2: 1-(3-methyl-6-(2-(2-methyl-6-(trifluoromethyl)pyrimidin-4-yl)-2,6-diazaspiro[3.4]octan-6-yl)-1H-pyrazolo[3,4-b]pyrazin-1-yl)propan-2-one: Followed the general procedure I using 1-(6-chloro-3-methyl-1H-pyrazolo[3,4-b]pyrazin-1-yl)propan-2-one (50 mg, 0.223 mmol, 1.0 equiv.) and 2-[2-methyl-6-(trifluoromethyl)pyrimidin-4-yl]-2,6-diazaspiro[3.4]octane (72.7 mg, 0.268 mmol, 1.2 equiv.) as the starting materials to give 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-b]pyrazine (100 mg, 97%) as a yellow oil. MS m/z: 461 [M+H]⁺.

Step 3: 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-b]pyrazine: Followed the general procedure AD using 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-b]pyrazine (150 mg, 0.326 mmol, 1.0 equiv.) as the starting material to give 1-(2,2-difluoropropyl)-3-methyl-6-(2-(2-methyl-6-(trifluoromethyl)pyrimidin-4-yl)-2,6-diazaspiro[3.4]octan-6-yl)-1H-pyrazolo[3,4-b]pyrazine (10.3 mg, 6.5%) as a white solid. ¹H NMR (400 MHz, DMSO-d₆) δ 8.01 (s, 1H), 6.66 (s, 1H), 4.60 (t, J=12.8 Hz, 2H), 4.21-4.08 (m, 4H), 3.84 (s, 2H), 3.65 (t, J=7.0 Hz, 2H), 2.45-2.39 (m, 6H), 2.30 (t, J=6.9 Hz, 2H), 1.64 (t, J=19.1 Hz, 3H). MS m/z: 483.30 [M+H]⁺.

### Example 1129: 1-(2,2-difluoropropyl)-3-methyl-6-(2-(6-methyl-2-(trifluoromethyl)pyrimidin-4-yl)-2,6-diazaspiro[3.4]octan-6-yl)-1H-pyrazolo[3,4-b]pyrazine

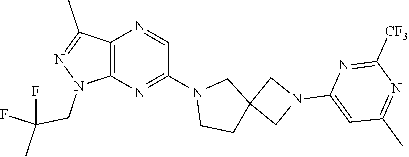 [SMILES: Cc1cc(N2CC3(CCN(c4cnc5c(C)nn(CC(C)(F)F)c5n4)C3)C2)nc(C(F)(F)F)n1]

Step 1; 1-(3-methyl-6-(2-(6-methyl-2-(trifluoromethyl)pyrimidin-4-yl)-2,6-diazaspiro[3.4]octan-6-yl)-1H-pyrazolo[3,4-b]pyrazin-1-yl)propan-2-one: Followed the general procedure I using 2-(6-methyl-2-(trifluoromethyl)pyrimidin-4-yl)-2,6-diazaspiro[3.4]octane (50 mg, 223 μmol, 1.0 equiv.) and 1-(6-chloro-3-methyl-1H-pyrazolo[3,4-b]pyrazin-1-yl)propan-2-one (72.7 mg, 268 μmol, 1.2 equiv.) as the starting materials to give 1-(3-methyl-6-(2-(6-methyl-2-(trifluoromethyl)pyrimidin-4-yl)-2,6-diazaspiro[3.4]octan-6-yl)-1H-pyrazolo[3,4-b]pyrazin-1-yl)propan-2-one (90 mg, 88%) as a white solid. MS m/z: 461 [M+H]⁺.

Step 2: 1-(2,2-difluoropropyl)-3-methyl-6-(2-(6-methyl-2-(trifluoromethyl)pyrimidin-4-yl)-2,6-diazaspiro[3.4]octan-6-yl)-1H-pyrazolo[3,4-b]pyrazine: Followed the general procedure AB using 1-(3-methyl-6-(2-(6-methyl-2-(trifluoromethyl)pyrimidin-4-yl)-2,6-diazaspiro[3.4]octan-6-yl)-1H-pyrazolo[3,4-b]pyrazin-1-yl)propan-2-one (65 mg, 141 μmol, 1.0 equiv.) as the starting material to give 1-(2,2-difluoropropyl)-3-methyl-6-(2-(6-methyl-2-(trifluoromethyl)pyrimidin-4-yl)-2,6-diazaspiro[3.4]octan-6-yl)-1H-pyrazolo[3,4-b]pyrazine (16.7 mg, 24%) as a white solid. ¹H NMR (300 MHz, DMSO-d₆) δ 8.00 (s, 1H), 6.50 (s, 1H), 4.60 (t, J=12.8 Hz, 2H), 4.12 (q, J=9.1 Hz, 4H), 3.85 (s, 2H), 3.65 (t, J=6.8 Hz, 2H), 2.42 (s, 3H), 2.32 (d, J=11.6 Hz, 5H), 1.64 (t, J=19.2 Hz, 3H). MS m/z: 483.25 [M+H]⁺.

### Example 1130: 1-(2,2-difluoropropyl)-3-methyl-6-(2-(6-methyl-2-(trifluoromethyl)pyrimidin-4-yl)-2,6-diazaspiro[3.4]octan-6-yl)-1H-pyrazolo[3,4-d]pyrimidine

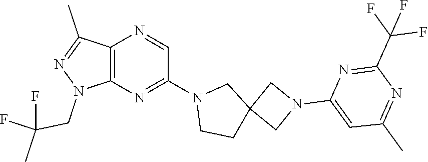 [SMILES: Cc1cc(N2CC3(CCN(c4cnc5c(C)nn(CC(C)(F)F)c5n4)C3)C2)nc(C(F)(F)F)n1]

Step 1: 1-(3-methyl-6-(2-(6-methyl-2-(trifluoromethyl)pyrimidin-4-yl)-2,6-diazaspiro[3.4]octan-6-yl)-1H-pyrazolo[3,4-d]pyrimidin-1-yl)propan-2-one: Followed the general procedure I using 1-(6-chloro-3-methyl-1H-pyrazolo[3,4-d]pyrimidin-1-yl)propan-2-one (50 mg, 0.223 mmol, 1.0 equiv.) and 2-(6-methyl-2-(trifluoromethyl)pyrimidin-4-yl)-2,6-diazaspiro[3.4]octane (72.7 mg, 0.268 mmol, 1.2 equiv.) as the starting materials to give 1-(3-methyl-6-(2-(6-methyl-2-(trifluoromethyl)pyrimidin-4-yl)-2,6-diazaspiro[3.4]octan-6-yl)-1H-pyrazolo[3,4-d]pyrimidin-1-yl)propan-2-one (70 mg, 68%) as a white solid. MS m/z: 460 [M+H]⁺.

Step 2: 1-(2,2-difluoropropyl)-3-methyl-6-(2-(6-methyl-2-(trifluoromethyl)pyrimidin-4-yl)-2,6-diazaspiro[3.4]octan-6-yl)-1H-pyrazolo[3,4-d]pyrimidine: Followed the general procedure AD using 1-(3-methyl-6-(2-(6-methyl-2-(trifluoromethyl)pyrimidin-4-yl)-2,6-diazaspiro[3.4]octan-6-yl)-1H-pyrazolo[3,4-d]pyrimidin-1-yl)propan-2-one (60 mg, 0.13 mmol, 1 equiv.) as the starting material to give 1-(2,2-difluoropropyl)-3-methyl-6-(2-(6-methyl-2-(trifluoromethyl)pyrimidin-4-yl)-2,6-diazaspiro[3.4]octan-6-yl)-1H-pyrazolo[3,4-d]pyrimidine (12.8 mg, 20%) as a white solid. ¹H NMR (400 MHz, DMSO-d₆) δ 8.88 (s, 1H), 6.49 (s, 1H), 4.57 (t, J=12.8 Hz, 2H), 4.18-4.08 (m, 2H), 4.08-4.00 (m, 2H), 3.83 (br, 2H), 3.65 (br, 2H), 2.42 (s, 3H), 2.34 (s, 3H), 2.26 (t, J=6.9 Hz, 2H), 1.65 (t, J=19.1 Hz, 3H). MS m/z: 483.20 [M+H]⁺.

### Example 1131: 2-(2-(2-(2-methyl-6-(trifluoromethyl)pyrimidin-4-yl)-2,6-diazaspiro[3.4]octan-6-yl)pyrimidin-4-yl)-1,3,4-thiadiazole

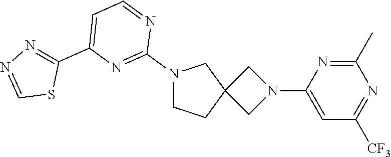 [SMILES: Cc1nc(N2CC3(CCN(c4nccc(-c5nncs5)n4)C3)C2)cc(C(F)(F)F)n1]

Step 1: methyl 2-(2-(2-methyl-6-(trifluoromethyl)pyrimidin-4-yl)-2,6-diazaspiro[3.4]octan-6-yl)pyrimidine-4-carboxylate: Followed the general procedure I using methyl 2-chloropyrimidine-4-carboxylate (300 mg, 1.73 mmol, 1.0 equiv.) and 2-(2-methyl-6-(trifluoromethyl)pyrimidin-4-yl)-2,6-diazaspiro[3.4]octane (521 mg, 1.90 mmol, 1.1 equiv.) as the starting materials to give methyl 2-(2-(2-methyl-6-(trifluoromethyl)pyrimidin-4-yl)-2,6-diazaspiro[3.4]octan-6-yl)pyrimidine-4-carboxylate (300 mg, 42%) as a white solid. MS m/z: 409 [M+H]⁺.

Step 2: 2-(2-(2-methyl-6-(trifluoromethyl)pyrimidin-4-yl)-2,6-diazaspiro[3.4]octan-6-yl)pyrimidine-4-carbohydrazide: Followed the general procedure S using methyl 2-(2-(2-methyl-6-(trifluoromethyl)pyrimidin-4-yl)-2,6-diazaspiro[3.4]octan-6-yl)pyrimidine-4-carboxylate (300 mg, 733 μmol, 1.0 equiv.) as the starting material to give 2-(2-(2-methyl-6-(trifluoromethyl)pyrimidin-4-yl)-2,6-diazaspiro[3.4]octan-6-yl)pyrimidine-4-carbohydrazide (250 mg, 83%) as a white solid. MS m/z: 409 [M+H]⁺

Step 3: N′-formyl-2-(2-(2-methyl-6-(trifluoromethyl)pyrimidin-4-yl)-2,6-diazaspiro[3.4]octan-6-yl)pyrimidine-4-carbohydrazide: Followed the general procedure AF using 2-(2-(2-methyl-6-(trifluoromethyl)pyrimidin-4-yl)-2,6-diazaspiro[3.4]octan-6-yl)pyrimidine-4-carbohydrazide (250 mg, 611 μmol, 1.0 equiv.) as the starting material to give N′-formyl-2-(2-(2-methyl-6-(trifluoromethyl)pyrimidin-4-yl)-2,6-diazaspiro[3.4]octan-6-yl)pyrimidine-4-carbohydrazide (200 mg, 74%) as a colorless oil. MS m/z: 437 [M+H]⁺

Step 4: 2-(2-(2-(2-methyl-6-(trifluoromethyl)pyrimidin-4-yl)-2,6-diazaspiro[3.4]octan-6-yl)pyrimidin-4-yl)-1,3,4-thiadiazole: Followed the general procedure T using N′-formyl-2-(2-(2-methyl-6-(trifluoromethyl)pyrimidin-4-yl)-2,6-diazaspiro[3.4]octan-6-yl)pyrimidine-4-carbohydrazide (200 mg, 457 μmol, 1.0 equiv.) as the starting material to give 2-(2-(2-(2-methyl-6-(trifluoromethyl)pyrimidin-4-yl)-2,6-diazaspiro[3.4]octan-6-yl)pyrimidin-4-yl)-1,3,4-thiadiazole (96.4 mg, 48%) as a white solid. ¹H NMR (400 MHz, DMSO-d₆) δ 9.76 (s, 1H), 8.61 (d, J=4.9 Hz, 1H), 7.41 (d, J=4.9 Hz, 1H), 6.64 (s, 1H), 4.23-4.08 (m, 4H), 3.84-3.79 (m, 2H), 3.64 (t, J=6.9 Hz, 2H), 2.43 (s, 3H), 2.27 (t, J=6.9 Hz, 2H) MS m/z: 435.20 [M+H]⁺.

### Example 1132: (R)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine, assumed; Example 1133: (S)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine, assumed

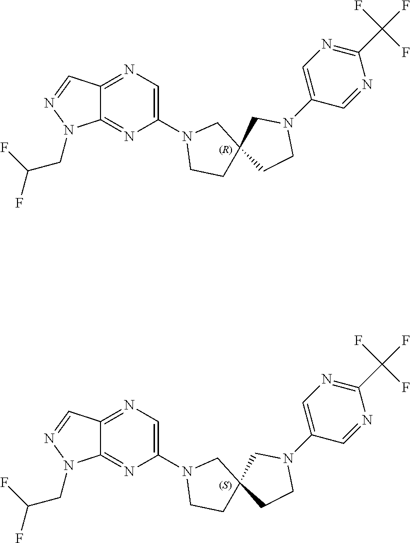

Step 1: (R)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine; (S)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine. The crude racemate (100 mg) was purified by Chiral-HPLC with the following method: Column: Lux Sum i-Cellulose-5, 3*25 cm, 5 m; Mobile Phase A: Hex (10 mM NH3-MeOH), Mobile Phase B: EtOH—HPLC; Flow rate: 20 mL/min; Gradient: 50% B to 50% B in 17.6 min; Wave Length: 195/273 nm; RT1(min): 8.7; RT2(min): 13; Sample Solvent: HFIP; Injection Volume: 0.8 mL; Number Of Runs: 7. This resulted in (R)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine (36.5 mg, 36%) and (S)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine (26.5 mg, 26%) as white solids.

(R)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine, assumed ¹H NMR (400 MHz, DMSO-d₆) δ 8.24 (s, 2H), 8.13 (s, 1H), 8.10 (s, 1H), 6.44 (tt, J=54.9, 3.8 Hz, 1H), 4.68 (td, J=15.0, 3.9 Hz, 2H), 3.73 (dt, J=18.3, 8.9 Hz, 3H), 3.66-3.58 (m, 2H), 3.53 (d, J=9.9 Hz, 2H), 3.45 (d, J=10.2 Hz, 2H), 2.16-2.06 (m, 4H). MS m/z: 455.15 [M+H]⁺.

(R)-1-(2,2-difluoroethyl)-6-(7-(2-(trifluoromethyl)pyrimidin-5-yl)-2,7-diazaspiro[4.4]nonan-2-yl)-1H-pyrazolo[3,4-b]pyrazine, assumed ¹H NMR (400 MHz, DMSO-d₆) δ 8.24 (s, 2H), 8.12 (d, J=11.1 Hz, 2H), 6.38 (dt, J=54.9, 3.8 Hz, 1H), 4.68 (td, J=14.9, 3.8 Hz, 2H), 3.77-3.70 (m, 1H), 3.69-3.61 (m, 2H), 3.57 (q, J=6.9 Hz, 2H), 3.52-3.43 (m, 2H), 2.17-2.05 (m, 4H). MS m/z: 455.10 [M+H]⁺.

### Example 1134: 1-(2,2-difluoropropyl)-6-(2-(6-methyl-2-(trifluoromethyl)pyrimidin-4-yl)-2,6-diazaspiro[3.4]octan-6-yl)-1H-pyrazolo[3,4-b]pyrazine

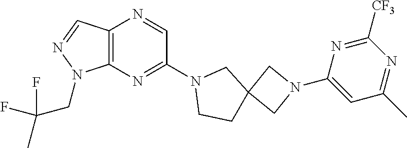 [SMILES: Cc1cc(N2CC3(CCN(c4cnc5cnn(CC(C)(F)F)c5n4)C3)C2)nc(C(F)(F)F)n1]

Step 1: 1-(6-(2-(6-methyl-2-(trifluoromethyl)pyrimidin-4-yl)-2,6-diazaspiro[3.4]octan-6-yl)-1H-pyrazolo[3,4-b]pyrazin-1-yl)propan-2-one: Followed the general procedure I using 1-(6-chloro-1H-pyrazolo[3,4-b]pyrazin-1-yl)propan-2-one (40 mg, 190 μmol, 1.0 equiv.) and 2-(6-methyl-2-(trifluoromethyl)pyrimidin-4-yl)-2,6-diazaspiro[3.4]octane (62.1 mg, 228 μmol, 1.2 equiv.) as the starting materials to give 1-(6-(2-(6-methyl-2-(trifluoromethyl)pyrimidin-4-yl)-2,6-diazaspiro[3.4]octan-6-yl)-1H-pyrazolo[3,4-b]pyrazin-1-yl)propan-2-one (80 mg, 94%) as a white solid. MS m/z: 447 [M+H]⁺.

Step 2: 1-(2,2-difluoropropyl)-6-(2-(6-methyl-2-(trifluoromethyl)pyrimidin-4-yl)-2,6-diazaspiro[3.4]octan-6-yl)-1H-pyrazolo[3,4-b]pyrazine: Followed the general procedure AB using 1-(6-(2-(6-methyl-2-(trifluoromethyl)pyrimidin-4-yl)-2,6-diazaspiro[3.4]octan-6-yl)-1H-pyrazolo[3,4-b]pyrazin-1-yl)propan-2-one (82 mg, 184 μmol, 1.0 equiv.) as the starting material to give 1-(2,2-difluoropropyl)-6-(2-(6-methyl-2-(trifluoromethyl)pyrimidin-4-yl)-2,6-diazaspiro[3.4]octan-6-yl)-1H-pyrazolo[3,4-b]pyrazine (23.3 mg, 27%) as a white solid. ¹H NMR (300 MHz, DMSO-d₆) δ 8.11 (d, J=13.7 Hz, 2H), 6.50 (s, 1H), 4.69 (t, J=12.8 Hz, 2H), 4.12 (q, J=9.1 Hz, 4H), 3.86 (s, 2H), 3.67 (t, J=6.9 Hz, 2H), 2.33 (d, J=10.6 Hz, 5H), 1.65 (t, J=19.1 Hz, 3H). MS m/z: 469.25 [M+H]⁺.

### Example 1135: 1-(2,2-difluoropropyl)-6-(2-(2-methyl-6-(trifluoromethyl)pyrimidin-4-yl)-2,6-diazaspiro[3.4]octan-6-yl)-1H-pyrazolo[3,4-d]pyrimidine

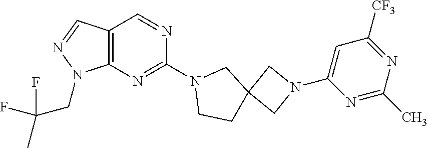 [SMILES: Cc1nc(N2CC3(CCN(c4ncc5cnn(CC(C)(F)F)c5n4)C3)C2)cc(C(F)(F)F)n1]

Step 1: 1-(6-chloro-1H-pyrazolo[3,4-d]pyrimidin-1-yl)propan-2-one: Followed the general procedure K using 6-chloro-1H-pyrazolo[3,4-d]pyrimidine (200 mg, 1.29 mmol, 1.0 equiv.) and bromoacetone (265 mg, 1.94 mmol, 1.5 equiv.) as the starting materials to give 1-(6-chloro-1H-pyrazolo[3,4-d]pyrimidin-1-yl)propan-2-one (100 mg, 36%) as a yellow solid. MS m/z: 211 [M+H]⁺.

Step 2: 1-(6-(2-(6-methyl-2-(trifluoromethyl)pyrimidin-4-yl)-2,6-diazaspiro[3.4]octan-6-yl)-1H-pyrazolo[3,4-d]pyrimidin-1-yl)propan-2-one: Followed the general procedure I using 1-(6-chloro-1H-pyrazolo[3,4-d]pyrimidin-1-yl)propan-2-one (40 mg, 0.19 mmol, 1.0 equiv.) and 2-[6-methyl-2-(trifluoromethyl)pyrimidin-4-yl]-2,6-diazaspiro[3.4]octane (62 mg, 0.228 mmol, 1.2 equiv.) as the starting materials to give 1-(6-(2-(6-methyl-2-(trifluoromethyl)pyrimidin-4-yl)-2,6-diazaspiro[3.4]octan-6-yl)-1H-pyrazolo[3,4-d]pyrimidin-1-yl)propan-2-one (80 mg, 94%) as a yellow oil. MS m/z: 447 [M+H]⁺.

Step 3: 1-(2,2-difluoropropyl)-6-(2-(2-methyl-6-(trifluoromethyl)pyrimidin-4-yl)-2,6-diazaspiro[3.4]octan-6-yl)-1H-pyrazolo[3,4-d]pyrimidine: Followed the general procedure AB using 1-(6-(2-(6-methyl-2-(trifluoromethyl)pyrimidin-4-yl)-2,6-diazaspiro[3.4]octan-6-yl)-1H-pyrazolo[3,4-d]pyrimidin-1-yl)propan-2-one (100 mg, 0.224 mmol, 1.0 equiv.) as the starting material to give 1-(2,2-difluoropropyl)-6-(2-(2-methyl-6-(trifluoromethyl)pyrimidin-4-yl)-2,6-diazaspiro[3.4]octan-6-yl)-1H-pyrazolo[3,4-d]pyrimidine (15 mg, 14%) as a white solid. ¹H NMR (300 MHz, DMSO-d₆) δ 8.92 (s, 1H), 8.09 (s, 1H), 6.65 (s, 1H), 4.67 (t, J=12.8 Hz, 2H), 4.14 (q, J=9.3 Hz, 4H), 3.84 (br, 2H), 3.67 (br, 2H), 2.43 (s, 3H), 2.27 (t, J=6.8 Hz, 2H), 1.66 (t, J=19.1 Hz, 3H). MS m/z: 469.25 [M+H]⁺.

### Example 1136: 1-ethyl-6-(2-(2-methyl-6-(trifluoromethyl)pyrimidin-4-yl)-2,6-diazaspiro[3.4]octan-6-yl)-3-(trifluoromethyl)-1H-pyrazolo[3,4-b]pyrazine

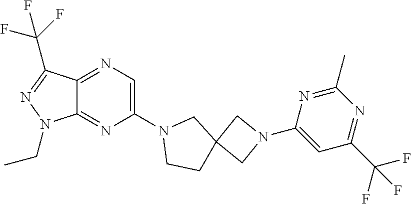 [SMILES: CCn1nc(C(F)(F)F)c2ncc(N3CCC4(CN(c5cc(C(F)(F)F)nc(C)n5)C4)C3)nc21]

Step 1: 6-chloro-3-(trifluoromethyl)-1H-pyrazolo[3,4-b]pyrazine: Followed the general procedure H using 6-chloro-1-(4-methoxybenzyl)-3-(trifluoromethyl)-1H-pyrazolo[3,4-b]pyrazine (400 mg) as the starting material to give 6-chloro-3-(trifluoromethyl)-1H-pyrazolo[3,4-b]pyrazine (200 mg). MS m/z: 223 [M+H]⁺.

Step 2: 6-chloro-1-ethyl-3-(trifluoromethyl)-1H-pyrazolo[3,4-b]pyrazine: Followed the general procedure I using 6-chloro-3-(trifluoromethyl)-1H-pyrazolo[3,4-b]pyrazine (200 mg, 0.90 μmol, 1.0 equiv.) and iodoethane (169 mg, 1.08 mmol, 1.1 equiv.) as the starting materials to give 6-chloro-1-ethyl-3-(trifluoromethyl)-1H-pyrazolo[3,4-b]pyrazine (100 mg, 44%). MS m/z: 251 [M+H]⁺.

Step 3: 1-ethyl-6-(2-(2-methyl-6-(trifluoromethyl)pyrimidin-4-yl)-2,6-diazaspiro[3.4]octan-6-yl)-3-(trifluoromethyl)-1H-pyrazolo[3,4-b]pyrazine: Followed the general procedure J using 6-chloro-1-ethyl-3-(trifluoromethyl)-1H-pyrazolo[3,4-b]pyrazine (100 mg, 0.40 mmol, 1.0 equiv.) and 2-(2-methyl-6-(trifluoromethyl)pyrimidin-4-yl)-2,6-diazaspiro[3.4]octane (131 mg, 0.48 mmol, 1.2 equiv.) as the starting materials to give 1-ethyl-6-(2-(2-methyl-6-(trifluoromethyl)pyrimidin-4-yl)-2,6-diazaspiro[3.4]octan-6-yl)-3-(trifluoromethyl)-1H-pyrazolo[3,4-b]pyrazine (86.9 mg, 45%) as a white solid. ¹H NMR (400 MHz, DMSO-d₆) δ 8.24 (s, 1H), 6.51 (s, 1H), 4.41-4.31 (m, 2H), 4.16 (d, J=9.0 Hz, 2H), 4.10 (d, J=9.2 Hz, 2H), 3.90 (s, 2H), 3.70 (s, 2H), 2.35 (s, 3H), 2.28 (s, 2H), 1.42 (t, J=7.2 Hz, 3H). MS m/z: 487.25 [M+H]⁺.

### Example 1137: 1-(2,2-difluoroethyl)-6-(2-(5-fluoro-2-methyl-6-(trifluoromethyl)pyrimidin-4-yl)-2,6-diazaspiro[3.4]octan-6-yl)-1H-pyrazolo[3,4-b]pyrazine

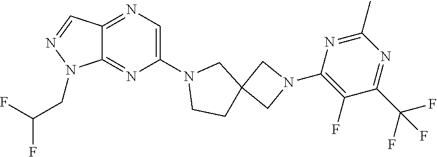 [SMILES: Cc1nc(N2CC3(CCN(c4cnc5cnn(CC(F)F)c5n4)C3)C2)c(F)c(C(F)(F)F)n1]

Step 1: 2-(5-fluoro-2-methyl-6-(trifluoromethyl)pyrimidin-4-yl)-2,6-diazaspiro[3.4]octane. To a stirred mixture of tert-butyl 2-(2-methyl-6-(trifluoromethyl)pyrimidin-4-yl)-2,6-diazaspiro[3.4]octane-6-carboxylate (200 mg, 0.537 mmol, 1.0 equiv.) in ACN (3 mL) was added selectfluor (380.52 mg, 1.074 mmol, 2.0 equiv.) in portions at room temperature under air atmosphere. The resulting mixture was stirred for 8 h at 80° C. under air atmosphere. The mixture was allowed to cool down to room temperature. The reaction was monitored by LCMS. Desired product could be detected by LCMS. The residue was purified by reversed-phase flash chromatography with the following conditions: column, C18 silica gel; mobile phase, MeCN in Water (0.1% FA), 5% to 100% gradient in 20 min; detector, UV 254 nm. This resulted in 2-(5-fluoro-2-methyl-6-(trifluoromethyl)pyrimidin-4-yl)-2,6-diazaspiro[3.4]octane (100 mg, 64%) as a white solid. MS m/z: 291 [M+H]⁺.

Step 2: 1-(2,2-difluoroethyl)-6-(2-(5-fluoro-2-methyl-6-(trifluoromethyl)pyrimidin-4-yl)-2,6-diazaspiro[3.4]octan-6-yl)-1H-pyrazolo[3,4-b]pyrazine: Followed the general procedure I using 2-(5-fluoro-2-methyl-6-(trifluoromethyl)pyrimidin-4-yl)-2,6-diazaspiro[3.4]octane (100 mg, 0.343 mmol, 1.0 equiv.) and 6-chloro-1-(2,2-difluoroethyl)-1H-pyrazolo[3,4-b]pyrazine (90 mg, 0.412 mmol, 1.2 equiv.) as the starting materials to give 1-(2,2-difluoroethyl)-6-(2-(5-fluoro-2-methyl-6-(trifluoromethyl)pyrimidin-4-yl)-2,6-diazaspiro[3.4]octan-6-yl)-1H-pyrazolo[3,4-b]pyrazine (106 mg, 65%) as a white solid. ¹H NMR (400 MHz, DMSO-d₆) δ 8.14 (s, 1H), 8.09 (s, 1H), 6.60-6.31 (m, 1H), 4.68 (td, J=14.9, 3.9 Hz, 2H), 4.27 (s, 4H), 3.87 (s, 2H), 3.67 (t, J=6.9 Hz, 2H), 2.40 (s, 3H), 2.32 (t, J=6.9 Hz, 2H). MS m/z: 473.25 [M+H]⁺.

### Example 1138: 1-(2,2-difluoroethyl)-6-(2-(5-fluoro-6-methyl-2-(trifluoromethyl)pyrimidin-4-yl)-2,6-diazaspiro[3.4]octan-6-yl)-1H-pyrazolo[3,4-b]pyrazine

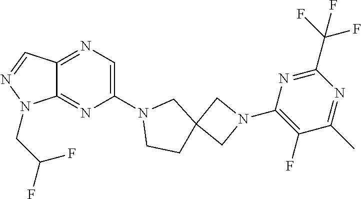 [SMILES: Cc1nc(C(F)(F)F)nc(N2CC3(CCN(c4cnc5cnn(CC(F)F)c5n4)C3)C2)c1F]

Step 1: 2-(5-fluoro-6-methyl-2-(trifluoromethyl)pyrimidin-4-yl)-2,6-diazaspiro[3.4]octane. A solution of tert-butyl 2-(6-methyl-2-(trifluoromethyl)pyrimidin-4-yl)-2,6-diazaspiro[3.4]octane-6-carboxylate (100 mg, 0.269 mmol, 1 equiv.) and selectfluor (190 mg, 0.538 mmol, 2 equiv.) in ACN (3 mL) was stirred for overnight at 80° C. Desired product could be detected by LCMS. The residue was purified by reversed-phase flash chromatography with the following conditions: column, C18 silica gel; mobile phase, MeCN in Water (0.1% NH₃H₂O), 0% to 100% gradient in 30 min; detector, UV 254 nm. The resulting mixture was concentrated under reduced pressure. This resulted in 2-(5-fluoro-6-methyl-2-(trifluoromethyl)pyrimidin-4-yl)-2,6-diazaspiro[3.4]octane (40 mg, 51.32%) as a white solid. MS m/z: 290 [M+H]⁺.

Step 2: 1-(2,2-difluoropropyl)-3-methyl-6-(2-(6-methyl-2-(trifluoromethyl)pyrimidin-4-yl)-2,6-diazaspiro[3.4]octan-6-yl)-1H-pyrazolo[3,4-d]pyrimidine: Followed the general procedure I using 2-(5-fluoro-6-methyl-2-(trifluoromethyl)pyrimidin-4-yl)-2,6-diazaspiro[3.4]octane (30 mg, 0.103 mmol, 1 equiv.) and 6-chloro-1-(2,2-difluoroethyl)-1H-pyrazolo[3,4-b]pyrazine (27.1 mg, 0.124 mmol, 1.2 equiv.) as the starting materials to give 1-(2,2-difluoroethyl)-6-(2-(5-fluoro-6-methyl-2-(trifluoromethyl)pyrimidin-4-yl)-2,6-diazaspiro[3.4]octan-6-yl)-1H-pyrazolo[3,4-b]pyrazine (7.3 mg, 15%) as a white solid. ¹H NMR (400 MHz, DMSO-d₆) δ 8.15-8.05 (m, 2H), 6.69-6.23 (m, 1H), 4.79-4.60 (m, 2H), 4.31 (br, 4H), 3.87 (s, 2H), 3.67 (t, J=6.8 Hz, 2H), 2.33 (d, J=3.2 Hz, 5H). MS m/z: 473.15 [M+H]⁺.

### Example 1139: 1-(2,2-difluoroethyl)-6-(2-(6-(difluoromethyl)-2-methylpyrimidin-4-yl)-2,6-diazaspiro[3.4]octan-6-yl)-1H-pyrazolo[3,4-b]pyrazine

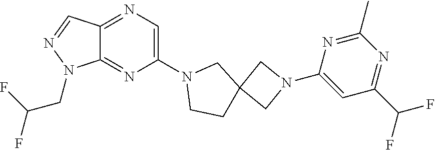 [SMILES: Cc1nc(C(F)F)cc(N2CC3(CCN(c4cnc5cnn(CC(F)F)c5n4)C3)C2)n1]

Step 1: 6-(difluoromethyl)-2-methylpyrimidin-4-ol. To a stirred solution of ethyl 4,4-difluoro-3-oxobutanoate (500 mg, 3.010 mmol, 1 equiv) and acetimidamide (192 mg, 3.31 mmol, 1.1 equiv.) in MeOH (4 mL) was added NaOMe (325 mg, 6.02 mmol, 2.0 equiv.) dropwise at 60° C. under argon atmosphere. The mixture was allowed to cool down to room temperature. The reaction was monitored by LCMS. Desired product could be detected by LCMS. The residue was purified by reversed-phase flash chromatography with the following conditions: column, C18 silica gel; mobile phase, MeCN in Water (0.1% FA), 5% to 100% gradient in 20 min; detector, UV 254 nm. This resulted in 6-(difluoromethyl)-2-methylpyrimidin-4-ol (400 mg, 82.17%) as a off-white oil. MS m/z: 161 [M+H]⁺.

Step 2: 4-chloro-6-(difluoromethyl)-2-methylpyrimidine: Followed the general procedure AE using 6-(difluoromethyl)-2-methylpyrimidin-4-ol (400 mg, 2.48 mmol, 1.0 equiv.) as the starting material to give 4-chloro-6-(difluoromethyl)-2-methylpyrimidine (300 mg, 67%) as a colorless liquid. MS m/z: 179 [M+H]⁺.

Step 3: tert-butyl 2-(6-(difluoromethyl)-2-methylpyrimidin-4-yl)-2,6-diazaspiro[3.4]octane-6-carboxylate: Followed the general procedure I using 4-chloro-6-(difluoromethyl)-2-methylpyrimidine (300 mg, 1.67 mmol, 1.0 equiv.) and tert-butyl 2,6-diazaspiro[3.4]octane-6-carboxylate (357 mg, 1.67 mmol, 1.0 equiv.) as the starting materials to give tert-butyl 2-(6-(difluoromethyl)-2-methylpyrimidin-4-yl)-2,6-diazaspiro[3.4]octane-6-carboxylate (200 mg, 33%) as a white solid. MS m/z: 355 [M+H]⁺.

Step 4: 2-(6-(difluoromethyl)-2-methylpyrimidin-4-yl)-2,6-diazaspiro[3.4]octane: Followed the general procedure H using tert-butyl 2-(6-(difluoromethyl)-2-methylpyrimidin-4-yl)-2,6-diazaspiro[3.4]octane-6-carboxylate (200 mg) as the starting material to give the crude product 2-(6-(difluoromethyl)-2-methylpyrimidin-4-yl)-2,6-diazaspiro[3.4]octane (160 mg). MS m/z: 255 [M+H]⁺.

Step 5: 1-(2,2-difluoroethyl)-6-(2-(6-(difluoromethyl)-2-methylpyrimidin-4-yl)-2,6-diazaspiro[3.4]octan-6-yl)-1H-pyrazolo[3,4-b]pyrazine: Followed the general procedure I using 2-(6-(difluoromethyl)-2-methylpyrimidin-4-yl)-2,6-diazaspiro[3.4]octane (160 mg, 627 μmol, 1.0 equiv.) and 6-chloro-1-(2,2-difluoroethyl)-1H-pyrazolo[3,4-b]pyrazine (152 mg, 690 μmol, 1.1 equiv.) as the starting materials to give 1-(2,2-difluoroethyl)-6-(2-(6-(difluoromethyl)-2-methylpyrimidin-4-yl)-2,6-diazaspiro[3.4]octan-6-yl)-1H-pyrazolo[3,4-b]pyrazine (71 mg, 26%) as a white solid. ¹H NMR (400 MHz, DMSO-d₆) δ 8.14 (s, 1H), 8.10 (s, 1H), 6.65 (d, J=45.2 Hz, 1H), 6.57-6.32 (m, 2H), 4.68 (td, J=14.9, 3.9 Hz, 2H), 4.14 (d, J=9.1 Hz, 2H), 4.09 (d, J=9.0 Hz, 2H), 3.86 (s, 2H), 3.67 (t, J=6.9 Hz, 2H), 2.40 (s, 3H), 2.30 (t, J=6.9 Hz, 2H). MS m/z: 437.25 [M+H]⁺.

### Example 1140: 1-(2,2-difluoroethyl)-6-(2-(2-(difluoromethyl)-6-methylpyrimidin-4-yl)-2,6-diazaspiro[3.4]octan-6-yl)-1H-pyrazolo[3,4-b]pyrazine

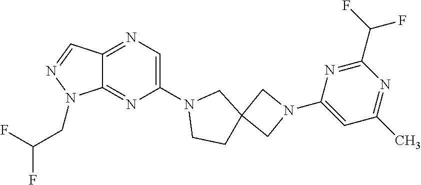 [SMILES: Cc1cc(N2CC3(CCN(c4cnc5cnn(CC(F)F)c5n4)C3)C2)nc(C(F)F)n1]

Step 1: tert-butyl 2-(6-chloro-2-(difluoromethyl)pyrimidin-4-yl)-2,6-diazaspiro[3.4]octane-6-carboxylate: Followed the general procedure I using tert-butyl 2,6-diazaspiro[3.4]octane-6-carboxylate hydrogen chloride (200 mg, 0.94 mmol, 1.0 equiv.) and 4,6-dichloro-2-(difluoromethyl)pyrimidine (187 mg, 0.94 mmol, 1.0 equiv.) as the starting materials to give tert-butyl 2-(6-chloro-2-(difluoromethyl)pyrimidin-4-yl)-2,6-diazaspiro[3.4]octane-6-carboxylate (200 mg, 57%) as a yellow solid. MS m/z: 375 [M+H]⁺.

Step 2: tert-butyl 2-(2-(difluoromethyl)-6-methylpyrimidin-4-yl)-2,6-diazaspiro[3.4]octane-6-carboxylate: Followed the general procedure I using tert-butyl 2-(6-chloro-2-(difluoromethyl)pyrimidin-4-yl)-2,6-diazaspiro[3.4]octane-6-carboxylate (200 mg, 0.54 mmol, 1.0 equiv.) and methylboronic acid (160 mg, 2.67 mmol, 5.0 equiv.) as the starting materials to give tert-butyl 2-(2-(difluoromethyl)-6-methylpyrimidin-4-yl)-2,6-diazaspiro[3.4]octane-6-carboxylate (180 mg, 55%) as a yellow solid. MS m/z: 355 [M+H]⁺.

Step 3: 2-(2-(difluoromethyl)-6-methylpyrimidin-4-yl)-2,6-diazaspiro[3.4]octane: Followed the general procedure H using tert-butyl 2-(2-(difluoromethyl)-6-methylpyrimidin-4-yl)-2,6-diazaspiro[3.4]octane-6-carboxylate (180 mg) as the starting material to give the crude product 2-(2-(difluoromethyl)-6-methylpyrimidin-4-yl)-2,6-diazaspiro[3.4]octane (150 mg). MS m/z: 255 [M+H]⁺.

Step 4: 1-(2,2-difluoroethyl)-6-(2-(2-(difluoromethyl)-6-methylpyrimidin-4-yl)-2,6-diazaspiro[3.4]octan-6-yl)-1H-pyrazolo[3,4-b]pyrazine: Followed the general procedure K using 2-(2-(difluoromethyl)-6-methylpyrimidin-4-yl)-2,6-diazaspiro[3.4]octane (150 mg, 0.59 mmol, 1.0 equiv.) and 6-chloro-1-(2,2-difluoroethyl)-1H-pyrazolo[3,4-b]pyrazine (154 mg, 0.71 mmol, 1.2 equiv.) as the starting materials to give 1-(2,2-difluoroethyl)-6-(2-(2-(difluoromethyl)-6-methylpyrimidin-4-yl)-2,6-diazaspiro[3.4]octan-6-yl)-1H-pyrazolo[3,4-b]pyrazine (130 mg, 50%) as a white solid. ¹H NMR (400 MHz, DMSO-d₆) δ 8.14 (s, 1H), 8.10 (s, 1H), 6.59 (s, 1H), 6.47-6.31 (m, 2H), 4.68 (td, J=14.8, 3.9 Hz, 2H), 4.16-4.04 (m, 4H), 3.86 (s, 2H), 3.67 (t, J=6.8 Hz, 2H), 2.32 (s, 5H). MS m/z: 437.25 [M+H]⁺.

### Example 1141: 7-methyl-9-(oxetan-3-yl)-2-(2-(2-(trifluoromethyl)pyridin-4-yl)-2,6-diazaspiro[3.4]octan-6-yl)-7,9-dihydro-8H-purin-8-one

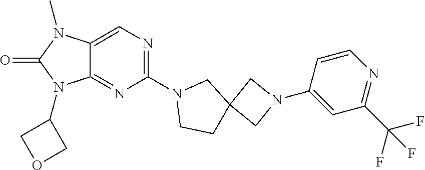 [SMILES: Cn1c(=O)n(C2COC2)c2nc(N3CCC4(CN(c5ccnc(C(F)(F)F)c5)C4)C3)ncc21]

Step 1: 2-chloro-5-nitro-N-(oxetan-3-yl)pyrimidin-4-amine. To a stirred solution of 2,4-dichloro-5-nitropyrimidine (3.5 g, 18 mmol, 1 equiv.) and oxetan-3-amine (1.98 g, 27 mmol, 1.5 equiv.) in THF (20 mL) was added DIEA (4.66 g, 36 mmol, 2 equiv.). The resulting mixture was stirred for 2 h at −78° C. Desired product could be detected by LCMS. The residue was purified by silica gel column chromatography, eluted with PE/EA (3:1) to afford 2-chloro-5-nitro-N-(oxetan-3-yl)pyrimidin-4-amine (1.3 g, 31%) as a white solid. MS m/z: 230 [M+H]⁺

Step 2: 2-chloro-N⁴-(oxetan-3-yl)pyrimidine-4,5-diamine. To a stirred solution of 2-chloro-5-nitro-N-(oxetan-3-yl)pyrimidin-4-amine (1.3 g, 5.63 mmol, 1 equiv.) and Fe (3.15 mg, 56.3 mmol, 10 equiv.) in NH₄Cl (0.25 mL), EtOH (12 mL). The resulting mixture was stirred for 3 h at 60° C. Desired product could be detected by LCMS. The reaction system was filtrated through celite and the filtrate was concentrated. The residue was purified by silica gel column chromatography, eluted with CH₂Cl₂/MeOH (10:1) to afford 2-chloro-N⁴-(oxetan-3-yl)pyrimidine-4,5-diamine (570 mg, 50%) as a yellow solid. MS m/z: 200 [M+H]⁺.

Step 3: 2-chloro-9-(oxetan-3-yl)-7,9-dihydro-8H-purin-8-one. To a stirred solution of 2-chloro-N⁴-(oxetan-3-yl)pyrimidine-4,5-diamine (570 mg, 2.84 mmol, 1 equiv.) in THF (6 mL) was added CDI (598 mg, 3.69 mmol, 1.3 equiv.). The resulting mixture was stirred for 2 h at 100° C. Desired product could be detected by LCMS. The residue was purified by silica gel column chromatography, eluted with PE/EA (1:1) to afford 2-chloro-9-(oxetan-3-yl)-7,9-dihydro-8H-purin-8-one (600 mg, 93%) as a yellow solid. MS m/z: 226 [M+H]⁺.

Step 4: 2-chloro-7-methyl-9-(oxetan-3-yl)-7,9-dihydro-8H-purin-8-one: Followed the general procedure K using 2-chloro-9-(oxetan-3-yl)-7,9-dihydro-8H-purin-8-one (600 mg, 2.64 mmol, 1 equiv.) and Mel (1127 mg, 7.94 mmol, 3 equiv.) as the starting materials to give 2-chloro-7-methyl-9-(oxetan-3-yl)-7,9-dihydro-8H-purin-8-one (580 mg, 91%) as a white solid. MS m/z: 240 [M+H]⁺.

Step 5: 7-methyl-9-(oxetan-3-yl)-2-(2-(2-(trifluoromethyl)pyridin-4-yl)-2,6-diazaspiro[3.4]octan-6-yl)-7,9-dihydro-8H-purin-8-one: Followed the general procedure I using 2-chloro-7-methyl-9-(oxetan-3-yl)-7,9-dihydro-8H-purin-8-one (150 mg, 0.623 mmol, 1 equiv.) and 2-(2-(trifluoromethyl)pyridin-4-yl)-2,6-diazaspiro[3.4]octane hydrochloride (208 mg, 0.81 mmol, 1.3 equiv.) as the starting materials to give 7-methyl-9-(oxetan-3-yl)-2-(2-(2-(trifluoromethyl)pyridin-4-yl)-2,6-diazaspiro[3.4]octan-6-yl)-7,9-dihydro-8H-purin-8-one (64.7 mg, 22%) as a white solid. ¹H NMR (400 MHz, DMSO-d₆) δ 8.24 (d, J=5.7 Hz, 1H), 8.07 (s, 1H), 6.75 (d, J=2.3 Hz, 1H), 6.61-6.51 (m, 1H), 5.52-5.36 (m, 1H), 5.38-5.28 (m, 2H), 4.83-4.69 (m, 2H), 4.10-3.93 (m, 4H), 3.74 (s, 2H), 3.56 (t, J=6.8 Hz, 2H), 3.27 (s, 3H), 2.24 (t, J=6.8 Hz, 2H). MS m/z: 462.30 [M+H]⁺.

### Example 1142: 9-(2,2-difluoroethyl)-7-methyl-2-(2-(2-(trifluoromethyl)pyridin-4-yl)-2,6-diazaspiro[3.4]octan-6-yl)-7,9-dihydro-8H-purin-8-one

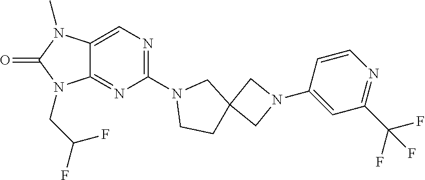 [SMILES: Cn1c(=O)n(CC(F)F)c2nc(N3CCC4(CN(c5ccnc(C(F)(F)F)c5)C4)C3)ncc21]

Step 1: 9-(2,2-difluoroethyl)-7-methyl-2-(2-(2-(trifluoromethyl)pyridin-4-yl)-2,6-diazaspiro[3.4]octan-6-yl)-7,9-dihydro-8H-purin-8-one: Followed the general procedure I using 2-chloro-9-(2,2-difluoroethyl)-7-methyl-7,9-dihydro-8H-purin-8-one (200 mg, 0.804 mmol, 1.0 equiv.) and 2-(2-(trifluoromethyl)pyridin-4-yl)-2,6-diazaspiro[3.4]octane (269 mg, 1.05 mmol, 1.3 equiv.) as the starting materials to give 9-(2,2-difluoroethyl)-7-methyl-2-(2-(2-(trifluoromethyl)pyridin-4-yl)-2,6-diazaspiro[3.4]octan-6-yl)-7,9-dihydro-8H-purin-8-one (107 mg, 28%) as a white solid. ¹H NMR (400 MHz, DMSO-d₆) δ 8.24 (d, J=5.7 Hz, 1H), 8.09 (s, 1H), 6.75 (d, J=2.2 Hz, 1H), 6.59-6.55 (m, 1H), 6.39 (tt, J=55.1, 3.9 Hz, 1H), 4.25-4.11 (m, 2H), 4.01 (q, J=8.3 Hz, 4H), 3.73 (s, 2H), 3.55 (t, J=6.8 Hz, 2H), 3.30 (s, 3H), 2.23 (t, J=6.8 Hz, 2H). MS m/z: 470.15 [M+H]⁺.

### Example 1143: 3-methyl-6-(2-(6-methyl-2-(trifluoromethyl)pyrimidin-4-yl)-2,6-diazaspiro[3.4]octan-6-yl)-1-(oxetan-3-yl)-1H-pyrazolo[3,4-b]pyrazine

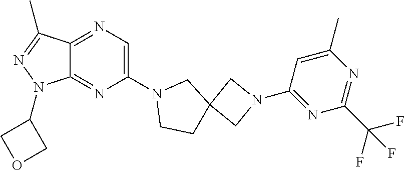 [SMILES: Cc1cc(N2CC3(CCN(c4cnc5c(C)nn(C6COC6)c5n4)C3)C2)nc(C(F)(F)F)n1]

Step 1: tert-butyl 2-(6-methyl-2-(trifluoromethyl)pyrimidin-4-yl)-2,6-diazaspiro[3.4]octane-6-carboxylate: Followed the general procedure I using 4-chloro-6-methyl-2-(trifluoromethyl)pyrimidine (10 g, 51 mmol, 1.0 equiv.) and tert-butyl 2,6-diazaspiro[3.4]octane-6-carboxylate (10.8 g, 51 mmol, 1.0 equiv.) as the starting materials to give tert-butyl 2-(6-methyl-2-(trifluoromethyl)pyrimidin-4-yl)-2,6-diazaspiro[3.4]octane-6-carboxylate (17 g, 89%) as a white solid. MS m/z: 373 [M+H]⁺.

Step 2: 2-(6-methyl-2-(trifluoromethyl)pyrimidin-4-yl)-2,6-diazaspiro[3.4]octane hydrochloride: Followed the general procedure B using tert-butyl 2-(6-methyl-2-(trifluoromethyl)pyrimidin-4-yl)-2,6-diazaspiro[3.4]octane-6-carboxylate (17 g) as the starting material to give the crude product 2-(6-methyl-2-(trifluoromethyl)pyrimidin-4-yl)-2,6-diazaspiro[3.4]octane hydrochloride (15 g). MS m/z: 273 [M+H]⁺.

Step 3: 1-(3,5-dichloropyrazin-2-yl)ethan-1-one. To a stirred solution of 3,5-dichloropyrazine-2-carbonitrile (40 g, 230 mmol, 1.0 equiv) in Et₂O (1 L) were added Methylmagnesium bromide, 3 M solution in diethyl ether (91.9 mL, 276 mmol, 1.2 equiv) dropwise at −78° C. under argon atmosphere. After the addition was completed, the mixture was slowly warmed to rt and stirred at rt for 1 h. The reaction was quenched with Ice/HCl in water (1M, 1 L) at 0° C. The mixture were extracted with diethyl ether (3×300 mL), dried over anhydrous Na₂SO₄. After filtration, the filtrate was concentrated under reduced pressure. The residue was purified by silica gel column chromatography, eluted with PE/EA (10:1) to afford 1-(3,5-dichloropyrazin-2-yl)ethan-1-one (43 g, 98%) as a yellow liquid. MS m/z: 191 [M+H]⁺.

Step 4: 6-chloro-1-(4-methoxybenzyl)-3-methyl-1H-pyrazolo[3,4-b]pyrazine. A solution of 1-(3,5-dichloropyrazin-2-yl)ethan-1-one (43 g, 225 mmol, 1.0 equiv.) and (4-methoxybenzyl)hydrazine dihydrochloride (44.6 g, 236 mmol, 1.05 equiv.) in EtOH (300 mL) was stirred for overnight at room temperature. The resulting mixture was filtered, the filter cake was washed with PE/EA (4/1) (3×300 mL). Then, the resulting mixture was concentrated under vacuum. The residue was dissolved in NMP (500 mL) and 2,6-Dimethylpyridine (96.5 mL, 900 mmol, 4.0 equiv.). The mixture was allowed to cool down to room temperature. The resulting mixture was extracted with EtOAc (1.5 L). The combined organic layers were washed with HCl in Water (1 M) (3×500 mL), dried over anhydrous Na₂SO₄. After filtration, the filtrate was concentrated under reduced pressure. The residue was purified by silica gel column chromatography, eluted with PE/EA (5:1) to afford 6-chloro-1-(4-methoxybenzyl)-3-methyl-1H-pyrazolo[3,4-b]pyrazine (19 g, 29%) as an off-white solid. MS m/z: 289[M+H]⁺.

Step 5: 6-chloro-3-methyl-1H-pyrazolo[3,4-b]pyrazine. A solution of 6-chloro-1-(4-methoxybenzyl)-3-methyl-1H-pyrazolo[3,4-b]pyrazine (19 g, 65.8 mmol, 1.0 equiv.) and CAN (72.4 g, 132 mmol, 2.0 equiv.) in ACN (400 mL)/H₂O (400 mL) was stirred for 3 h at 60° C. The resulting mixture was concentrated under vacuum. The residue was purified by silica gel column chromatography, eluted with PE/EA (5:1) to afford 6-chloro-3-methyl-1H-pyrazolo[3,4-b]pyrazine (10 g, 90%) as a white solid. MS m/z: 169 [M+H]⁺.

Step 6: 6-chloro-3-methyl-1-(oxetan-3-yl)-1H-pyrazolo[3,4-b]pyrazine: Followed the general procedure K using 6-chloro-3-methyl-1H-pyrazolo[3,4-b]pyrazine (10 g, 59.2 mmol, 1.0 equiv.) and oxetan-3-yl trifluoromethanesulfonate (14.6 g, 71 mmol, 1.2 equiv.) as the starting materials to give 6-chloro-3-methyl-1-(oxetan-3-yl)-1H-pyrazolo[3,4-b]pyrazine (7.2 g, 54%) as a white solid. MS m/z: 225[M+H]⁺.

Step 7: 3-methyl-6-(2-(6-methyl-2-(trifluoromethyl)pyrimidin-4-yl)-2,6-diazaspiro[3.4]octan-6-yl)-1-(oxetan-3-yl)-1H-pyrazolo[3,4-b]pyrazine: Followed the general procedure I using 6-chloro-3-methyl-1-(oxetan-3-yl)-1H-pyrazolo[3,4-b]pyrazine (5.5 g, 24.4 mmol, 1.0 equiv.) and 2-(6-methyl-2-(trifluoromethyl)pyrimidin-4-yl)-2,6-diazaspiro[3.4]octane hydrochloride (8.3 g, 26.8 mmol, 1.1 equiv.) as the starting materials to give crude product. The crude product was purified by reversed-phase flash chromatography with the following conditions: column, C18 silica gel; mobile phase, MeCN in Water (0.1% NH₃·H₂O), 10% to 100% gradient in 40 min; detector, UV 254 nm. This resulted in 3-methyl-6-(2-(6-methyl-2-(trifluoromethyl)pyrimidin-4-yl)-2,6-diazaspiro[3.4]octan-6-yl)-1-(oxetan-3-yl)-1H-pyrazolo[3,4-b]pyrazine (8.5 g, 76%) as a white solid. ¹H NMR (400 MHz, DMSO-d₆) δ 7.99 (s, 1H), 6.50 (s, 1H), 5.81 (tt, J=7.9, 6.5 Hz, 1H), 5.05 (t, J=6.4 Hz, 2H), 4.94 (dd, J=7.9, 6.3 Hz, 2H), 4.11 (q, J=9.1 Hz, 4H), 3.84 (s, 2H), 3.65 (t, J=6.8 Hz, 2H), 2.46 (s, 3H), 2.35 (s, 3H), 2.30 (t, J=6.9 Hz, 2H). MS m/z: 461.2 [M+H]⁺.

### Example 1144: (R)-6-(2-(2-methyl-6-(trifluoromethyl)pyrimidin-4-yl)-2,6-diazaspiro[3.4]octan-6-yl)-1-(tetrahydrofuran-3-yl)-1H-pyrazolo[3,4-b]pyrazine

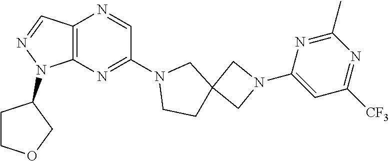 [SMILES: Cc1nc(N2CC3(CCN(c4cnc5cnn([C@@H]6CCOC6)c5n4)C3)C2)cc(C(F)(F)F)n1]

Step 1: (R)-6-(2-(2-methyl-6-(trifluoromethyl)pyrimidin-4-yl)-2,6-diazaspiro[3.4]octan-6-yl)-1-(tetrahydrofuran-3-yl)-1H-pyrazolo[3,4-b]pyrazine: Followed the general procedure I using (R)-6-chloro-1-(tetrahydrofuran-3-yl)-1H-pyrazolo[3,4-b]pyrazine (50 mg, 0.223 mmol, 1.0 equiv.) and 2-(2-methyl-6-(trifluoromethyl)pyrimidin-4-yl)-2,6-diazaspiro[3.4]octane (72.8 mg, 0.267 mmol, 1.2 equiv.) as the starting materials to give (R)-6-(2-(2-methyl-6-(trifluoromethyl)pyrimidin-4-yl)-2,6-diazaspiro[3.4]octan-6-yl)-1-(tetrahydrofuran-3-yl)-1H-pyrazolo[3,4-b]pyrazine (73.4 mg, 71%) as a white solid. ¹H NMR (400 MHz, DMSO-d₆) δ 8.07 (s, 2H), 6.66 (s, 1H), 5.39-5.28 (m, 1H), 4.22-4.02 (m, 6H), 3.95-3.87 (m, 2H), 3.86 (d, J=8.2 Hz, 2H), 3.66 (t, J=6.9 Hz, 2H), 2.43 (s, 3H), 2.41-2.35 (m, 2H), 2.30 (t, J=7.0 Hz, 2H). MS m/z: 461.30 [M+H]⁺.

### Example 1145: (S)-6-(2-(2-methyl-6-(trifluoromethyl)pyrimidin-4-yl)-2,6-diazaspiro[3.4]octan-6-yl)-1-(tetrahydrofuran-3-yl)-1H-pyrazolo[3,4-b]pyrazine

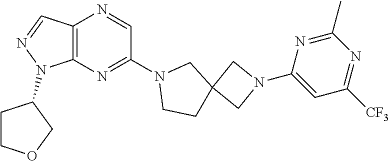 [SMILES: Cc1nc(N2CC3(CCN(c4cnc5cnn([C@H]6CCOC6)c5n4)C3)C2)cc(C(F)(F)F)n1]

Step 1: (S)-6-(2-(2-methyl-6-(trifluoromethyl)pyrimidin-4-yl)-2,6-diazaspiro[3.4]octan-6-yl)-1-(tetrahydrofuran-3-yl)-1H-pyrazolo[3,4-b]pyrazine: Followed the general procedure I using (S)-6-chloro-1-(tetrahydrofuran-3-yl)-1H-pyrazolo[3,4-b]pyrazine (50 mg, 0.223 mmol, 1.0 equiv.) and 2-(2-methyl-6-(trifluoromethyl)pyrimidin-4-yl)-2,6-diazaspiro[3.4]octane (61 mg, 0.223 mmol, 1.0 equiv.) as the starting materials to give (S)-6-(2-(2-methyl-6-(trifluoromethyl)pyrimidin-4-yl)-2,6-diazaspiro[3.4]octan-6-yl)-1-(tetrahydrofuran-3-yl)-1H-pyrazolo[3,4-b]pyrazine (89.8 mg, 85%) as a white solid. ¹H NMR (400 MHz, DMSO-d₆) δ 8.07 (d, J=1.6 Hz, 2H), 6.66 (s, 1H), 5.44-5.21 (m, 1H), 4.34-4.04 (m, 6H), 3.93-3.83 (m, 4H), 3.66 (t, J=6.9 Hz, 2H), 2.71-2.53 (m, 3H), 2.43 (s, 2H), 2.40-2.31 (m, 2H). MS m/z: 461.2 [M+H]⁺.

### Example 1146: (S)-6-(2-(6-methyl-2-(trifluoromethyl)pyrimidin-4-yl)-2,6-diazaspiro[3.4]octan-6-yl)-1-(tetrahydrofuran-3-yl)-1H-pyrazolo[3,4-b]pyrazine

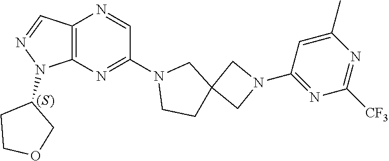

Step 1: 2-(6-methyl-2-(trifluoromethyl)pyrimidin-4-yl)-2,6-diazaspiro[3.4]octane hydrochloride: Followed the general procedure B using tert-butyl 2-(6-methyl-2-(trifluoromethyl)pyrimidin-4-yl)-2,6-diazaspiro[3.4]octane-6-carboxylate (200 mg) as the starting material to give the crude product 2-(6-methyl-2-(trifluoromethyl)pyrimidin-4-yl)-2,6-diazaspiro[3.4]octane hydrochloride (140 mg). MS m/z: 272 [M+H]⁺.

Step 2: (S)-6-(2-(6-methyl-2-(trifluoromethyl)pyrimidin-4-yl)-2,6-diazaspiro[3.4]octan-6-yl)-1-(tetrahydrofuran-3-yl)-1H-pyrazolo[3,4-b]pyrazine: Followed the general procedure I using 2-(6-methyl-2-(trifluoromethyl)pyrimidin-4-yl)-2,6-diazaspiro[3.4]octane (60 mg, 0.22 mmol, 1.0 equiv.) and (S)-6-chloro-1-(tetrahydrofuran-3-yl)-1H-pyrazolo[3,4-b]pyrazine (64.4 mg, 0.286 mmol, 1.3 equiv.) as the starting materials to give (S)-6-(2-(6-methyl-2-(trifluoromethyl)pyrimidin-4-yl)-2,6-diazaspiro[3.4]octan-6-yl)-1-(tetrahydrofuran-3-yl)-1H-pyrazolo[3,4-b]pyrazine (37.3 mg, 36%) as a white solid. ¹H NMR (300 MHz, DMSO-d₆) δ 8.10-8.02 (m, 2H), 6.50 (s, 1H), 5.43-5.19 (m, 1H), 4.26-4.02 (m, 6H), 3.94-3.82 (m, 4H), 3.66 (t, J=6.8 Hz, 2H), 2.42-2.27 (m, 7H). MS m/z: 461.30 [M+H]⁺.

### Example 1147: (R)-6-(2-(6-methyl-2-(trifluoromethyl)pyrimidin-4-yl)-2,6-diazaspiro[3.4]octan-6-yl)-1-(tetrahydrofuran-3-yl)-1H-pyrazolo[3,4-b]pyrazine

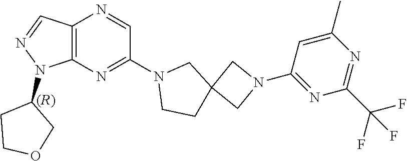

Step 1: (R)-6-(2-(6-methyl-2-(trifluoromethyl)pyrimidin-4-yl)-2,6-diazaspiro[3.4]octan-6-yl)-1-(tetrahydrofuran-3-yl)-1H-pyrazolo[3,4-b]pyrazine: Followed the general procedure I using 2-(6-methyl-2-(trifluoromethyl)pyrimidin-4-yl)-2,6-diazaspiro[3.4]octane (60 mg, 0.22 mmol, 1.0 equiv) and (R)-6-chloro-1-(tetrahydrofuran-3-yl)-1H-pyrazolo[3,4-b]pyrazine (54.4 mg, 0.242 mmol, 1.1 equiv) as the starting materials to give (R)-6-(2-(6-methyl-2-(trifluoromethyl)pyrimidin-4-yl)-2,6-diazaspiro[3.4]octan-6-yl)-1-(tetrahydrofuran-3-yl)-1H-pyrazolo[3,4-b]pyrazine (46.2 mg, 44%) as a white solid. ¹H NMR (400 MHz, DMSO-d₆) δ 8.10-8.02 (m, 2H), 6.50 (s, 1H), 5.43-5.23 (m, 1H), 4.28-4.02 (m, 6H), 3.95-3.81 (m, 4H), 3.73-3.60 (m, 2H), 2.42-2.36 (m, 2H), 2.34 (s, 3H), 2.33-2.28 (m, 2H). MS m/z: 461.30 [M+H]⁺.

### Biological Assay Data and Procedures

Exemplary compounds were evaluated for activation of GCase in a live cell PFB assay in HELA cells (essentially as described in Ysselstein et al., “LRRK2 kinase activity regulates lysosomal glucocerebrosidase in neurons derived from Parkinson's disease patients” Nature Communications (2019) 10:5570). The results in Table 2 demonstrate that compounds of the disclosure are potent activators of GCase. EC₅₀ ranges: A: <10 μM; B: >10-50 μM; C: >50-100 μM; D: >100 μM.

TABLE 2
In vitro enzymatic EC₅₀ values for exemplary compounds

| | GCase | | GCase | | GCase | | GCase | | GCase | | GCase |
|---|---|---|---|---|---|---|---|---|---|---|---|
| Cmpd | EC₅₀ | Cmpd | EC₅₀ | Cmpd | EC₅₀ | Cmpd | EC₅₀ | Cmpd | EC₅₀ | Cmpd | EC₅₀ |
| No. | (μM) | No. | (μM) | No. | (μM) | No. | (μM) | No. | (μM) | No. | (μM) |
| 1 | A | 234 | B | 434 | B | 634 | A | 834 | A | 1034 | A |
| 2 | A | 235 | C | 435 | A | 635 | A | 835 | A | 1035 | A |
| 3 | A | 236 | A | 436 | B | 636 | A | 836 | A | 1036 | D |
| 4 | A | 237 | B | 437 | C | 637 | B | 837 | A | 1037 | A |
| 5 | A | 238 | B | 438 | B | 638 | B | 838 | B | 1038 | B |
| 6 | A | 239 | A | 439 | B | 639 | B | 839 | A | 1039 | B |
| 7 | A | 240 | C | 440 | C | 640 | B | 840 | A | 1040 | B |
| 8 | A | 241 | B | 441 | A | 641 | B | 841 | A | 1041 | D |
| 9 | A | 242 | B | 442 | A | 642 | B | 842 | B | 1042 | D |
| 10 | A | 243 | A | 443 | A | 643 | B | 843 | B | 1043 | A |
| 11 | A | 244 | B | 444 | A | 644 | B | 844 | A | 1044 | A |
| 12 | B | 245 | B | 445 | B | 645 | B | 845 | B | 1045 | C |
| 13 | B | 246 | B | 446 | A | 646 | B | 846 | B | 1046 | B |
| 14 | B | 247 | A | 447 | A | 647 | B | 847 | B | 1047 | D |
| 15 | B | 248 | A | 448 | A | 648 | A | 848 | B | 1048 | B |
| 16 | B | 249 | B | 449 | A | 649 | B | 849 | A | 1049 | B |
| 17 | B | 250 | B | 450 | B | 650 | A | 850 | A | 1050 | B |
| 18 | B | 251 | A | 451 | B | 651 | B | 851 | A | 1051 | C |
| 19 | B | 252 | B | 452 | A | 652 | D | 852 | B | 1052 | A |
| 20 | B | 253 | A | 453 | B | 653 | D | 853 | B | 1053 | A |
| 21 | B | 254 | B | 454 | B | 654 | B | 854 | B | 1054 | A |
| 22 | B | 255 | A | 455 | B | 655 | B | 855 | B | 1055 | A |
| 23 | B | 256 | A | 456 | B | 656 | C | 856 | B | 1056 | B |
| 24 | B | 257 | A | 457 | B | 657 | B | 857 | A | 1057 | B |
| 25 | B | 258 | A | 458 | A | 658 | B | 858 | B | 1058 | B |
| 26 | B | 259 | B | 459 | B | 659 | B | 859 | B | 1059 | A |
| 27 | B | 260 | A | 460 | A | 660 | B | 860 | B | 1060 | A |
| 28 | B | 261 | B | 461 | D | 661 | B | 861 | A | 1061 | C |
| 29 | B | 262 | A | 462 | B | 662 | B | 862 | D | 1062 | B |
| 30 | B | 263 | A | 463 | B | 663 | B | 863 | A | 1063 | B |
| 31 | B | 264 | D | 464 | A | 664 | B | 864 | B | 1064 | B |
| 32 | B | 265 | B | 465 | D | 665 | D | 865 | B | 1065 | B |
| 33 | B | 266 | A | 466 | D | 666 | A | 866 | B | 1066 | B |
| 34 | B | 267 | B | 467 | A | 667 | C | 867 | D | 1067 | A |
| 35 | B | 268 | B | 468 | A | 668 | A | 868 | B | 1068 | B |
| 36 | B | 269 | B | 469 | D | 669 | B | 869 | B | 1069 | B |
| 37 | B | 270 | B | 470 | B | 670 | B | 870 | D | 1070 | A |
| 38 | B | 271 | B | 471 | A | 671 | A | 871 | B | 1071 | A |
| 39 | C | 272 | B | 472 | D | 672 | B | 872 | A | 1072 | B |
| 40 | C | 273 | B | 473 | B | 673 | A | 873 | B | 1073 | A |
| 41 | C | 274 | A | 474 | D | 674 | A | 874 | A | 1074 | A |
| 57 | B | 275 | A | 475 | D | 675 | B | 875 | A | 1075 | B |
| 58 | B | 276 | B | 476 | C | 676 | A | 876 | B | 1076 | B |
| 66 | A | 277 | B | 477 | A | 677 | B | 877 | A | 1077 | A |
| 68 | A | 278 | A | 478 | A | 678 | B | 878 | B | 1078 | A |
| 69 | B | 279 | A | 479 | D | 679 | B | 879 | A | 1079 | B |
| 70 | A | 280 | B | 480 | B | 680 | B | 880 | A | 1080 | B |
| 73 | A | 281 | A | 481 | B | 681 | B | 881 | A | 1081 | A |
| 75 | A | 282 | A | 482 | B | 682 | B | 882 | A | 1082 | A |
| 76 | A | 283 | A | 483 | B | 683 | A | 883 | B | 1083 | B |
| 77 | A | 284 | C | 484 | A | 684 | A | 884 | B | 1084 | A |
| 78 | B | 285 | B | 485 | D | 685 | B | 885 | B | 1085 | A |
| 79 | A | 286 | A | 486 | C | 686 | D | 886 | B | 1086 | B |
| 80 | B | 287 | D | 487 | B | 687 | A | 887 | B | 1087 | A |
| 81 | B | 288 | B | 488 | D | 688 | A | 888 | A | 1088 | A |
| 82 | B | 289 | B | 489 | B | 689 | B | 889 | A | 1089 | B |
| 83 | B | 290 | A | 490 | A | 690 | A | 890 | A | 1090 | D |
| 84 | B | 291 | A | 491 | B | 691 | B | 891 | B | 1091 | D |
| 85 | B | 292 | B | 492 | B | 692 | B | 892 | A | 1092 | D |
| 86 | A | 293 | B | 493 | A | 693 | A | 893 | A | 1093 | B |
| 87 | A | 294 | B | 494 | A | 694 | B | 894 | B | 1094 | A |
| 88 | A | 295 | B | 495 | B | 695 | A | 895 | A | 1095 | B |
| 89 | B | 296 | A | 496 | B | 696 | A | 896 | A | 1096 | A |
| 92 | B | 297 | B | 497 | C | 697 | B | 897 | A | 1097 | B |
| 95 | B | 298 | D | 498 | B | 698 | A | 898 | A | 1098 | A |
| 96 | B | 299 | B | 499 | A | 699 | A | 899 | A | 1099 | A |
| 97 | C | 300 | C | 500 | D | 700 | A | 900 | A | 1100 | B |
| 98 | C | 301 | B | 501 | D | 701 | B | 901 | A | 1101 | B |
| 99 | A | 302 | A | 502 | A | 702 | B | 902 | A | 1102 | A |
| 100 | B | 303 | A | 503 | A | 703 | A | 903 | A | 1103 | A |
| 101 | B | 304 | A | 504 | B | 704 | A | 904 | A | 1104 | B |
| 102 | B | 305 | B | 505 | D | 705 | B | 905 | B | 1105 | A |
| 103 | B | 306 | B | 506 | D | 706 | A | 906 | B | 1106 | A |
| 104 | B | 307 | A | 507 | A | 707 | B | 907 | A | 1107 | A |
| 108 | B | 308 | B | 508 | B | 708 | B | 908 | B | 1108 | A |
| 109 | B | 309 | B | 509 | B | 709 | B | 909 | B | 1109 | B |
| 110 | B | 310 | B | 510 | D | 710 | B | 910 | B | 1110 | C |
| 111 | B | 311 | B | 511 | A | 711 | B | 911 | B | 1111 | B |
| 112 | B | 312 | C | 512 | A | 712 | B | 912 | B | 1112 | A |
| 113 | D | 313 | B | 513 | B | 713 | A | 913 | B | 1113 | B |
| 114 | B | 314 | D | 514 | A | 714 | A | 914 | B | 1114 | A |
| 115 | B | 315 | B | 515 | A | 715 | A | 915 | A | 1115 | B |
| 116 | B | 316 | B | 516 | A | 716 | A | 916 | A | 1116 | B |
| 117 | A | 317 | B | 517 | A | 717 | A | 917 | C | 1117 | D |
| 118 | B | 318 | B | 518 | D | 718 | A | 918 | B | 1118 | D |
| 119 | A | 319 | B | 519 | B | 719 | A | 919 | A | 1119 | B |
| 120 | A | 320 | A | 520 | B | 720 | B | 920 | B | 1120 | D |
| 121 | A | 321 | A | 521 | A | 721 | D | 921 | B | 1121 | D |
| 122 | A | 322 | B | 522 | A | 722 | B | 922 | A | 1122 | B |
| 123 | B | 323 | D | 523 | A | 723 | B | 923 | A | 1123 | B |
| 124 | A | 324 | B | 524 | A | 724 | B | 924 | A | 1124 | C |
| 125 | B | 325 | B | 525 | A | 725 | B | 925 | B | 1125 | B |
| 126 | A | 326 | B | 526 | A | 726 | B | 926 | B | 1126 | A |
| 127 | A | 327 | B | 527 | A | 727 | A | 927 | B | 1127 | A |
| 128 | B | 328 | B | 528 | A | 728 | B | 928 | B | 1128 | B |
| 129 | B | 329 | B | 529 | A | 729 | A | 929 | B | 1129 | A |
| 130 | B | 330 | B | 530 | A | 730 | B | 930 | B | 1130 | A |
| 131 | A | 331 | B | 531 | A | 731 | B | 931 | B | 1131 | D |
| 132 | B | 332 | A | 532 | B | 732 | B | 932 | B | 1132 | B |
| 133 | A | 333 | A | 533 | A | 733 | A | 933 | A | 1133 | B |
| 134 | A | 334 | C | 534 | A | 734 | A | 934 | A | 1134 | D |
| 135 | C | 335 | B | 535 | A | 735 | B | 935 | C | 1135 | B |
| 136 | B | 336 | B | 536 | A | 736 | B | 936 | A | 1136 | A |
| 137 | D | 337 | A | 537 | D | 737 | A | 937 | A | 1137 | C |
| 138 | B | 338 | B | 538 | B | 738 | A | 938 | B | 1138 | B |
| 139 | B | 339 | B | 539 | B | 739 | A | 939 | A | 1139 | A |
| 140 | B | 340 | A | 540 | B | 740 | A | 940 | A | 1140 | A |
| 141 | B | 341 | B | 541 | A | 741 | A | 941 | A | 1141 | A |
| 142 | A | 342 | D | 542 | A | 742 | A | 942 | B | 1142 | A |
| 143 | A | 343 | D | 543 | B | 743 | B | 943 | A | 1143 | A |
| 144 | A | 344 | A | 544 | B | | | 944 | A | 1144 | A |
| 145 | B | 345 | A | 545 | D | 745 | A | 945 | B | 1145 | B |
| 146 | A | 346 | C | 546 | B | 746 | A | 946 | B | 1146 | B |
| 147 | A | 347 | B | 547 | B | 747 | B | 947 | A | 1147 | A |
| 148 | A | 348 | B | 548 | D | 748 | A | 948 | B | | |
| 149 | A | 349 | B | 549 | D | 749 | B | 949 | B | | |
| 150 | B | 350 | A | 550 | D | 750 | A | 950 | D | | |
| 151 | B | 351 | A | 551 | A | 751 | B | 951 | A | | |
| 152 | A | 352 | A | 552 | A | 752 | A | 952 | B | | |
| 153 | A | 353 | A | 553 | B | 753 | A | 953 | B | | |
| 154 | A | 354 | A | 554 | B | 754 | D | 954 | B | | |
| 155 | A | 355 | A | 555 | A | 755 | B | 955 | A | | |
| 156 | A | 356 | B | 556 | B | 756 | B | 956 | D | | |
| 157 | D | 357 | A | 557 | B | 757 | A | 957 | B | | |
| 158 | D | 358 | A | 558 | A | 758 | A | 958 | A | | |
| 159 | D | 359 | A | 559 | B | 759 | A | 959 | B | | |
| 160 | B | 360 | A | 560 | B | 760 | A | 960 | B | | |
| 161 | B | 361 | B | 561 | B | 761 | A | 961 | B | | |
| 162 | A | 362 | A | 562 | B | 762 | A | 962 | B | | |
| 163 | A | 363 | A | 563 | D | 763 | A | 963 | A | | |
| 164 | B | 364 | A | 564 | A | 764 | A | 964 | A | | |
| 165 | A | 365 | B | 565 | B | 765 | A | 965 | B | | |
| 166 | A | 366 | A | 566 | A | 766 | A | 966 | A | | |
| 167 | B | 367 | B | 567 | A | 767 | A | 967 | A | | |
| 168 | A | 368 | A | 568 | A | 768 | A | 968 | A | | |
| 169 | A | 369 | B | 569 | B | 769 | B | 969 | A | | |
| 170 | B | 370 | B | 570 | B | 770 | A | 970 | C | | |
| 171 | B | 371 | A | 571 | B | 771 | A | 971 | A | | |
| 172 | B | 372 | B | 572 | A | 772 | D | 972 | B | | |
| 173 | B | 373 | B | 573 | D | 773 | A | 973 | B | | |
| 174 | C | 374 | B | 574 | B | 774 | A | 974 | C | | |
| 175 | C | 375 | A | 575 | D | 775 | D | 975 | B | | |
| 176 | D | 376 | B | 576 | B | 776 | B | 976 | A | | |
| 177 | C | 377 | D | 577 | B | 777 | D | 977 | B | | |
| 178 | B | 378 | D | 578 | B | 778 | A | 978 | A | | |
| 179 | B | 379 | B | 579 | B | 779 | A | 979 | A | | |
| 180 | B | 380 | A | 580 | B | 780 | A | 980 | A | | |
| 181 | B | 381 | A | 581 | B | 781 | A | 981 | B | | |
| 182 | A | 382 | B | 582 | B | 782 | A | 982 | B | | |
| 183 | C | 383 | A | 583 | D | 783 | C | 983 | B | | |
| 184 | B | 384 | A | 584 | B | 784 | A | 984 | A | | |
| 185 | D | 385 | B | 585 | A | 785 | D | 985 | D | | |
| 186 | B | 386 | B | 586 | B | 786 | A | 986 | A | | |
| 187 | B | 387 | B | 587 | B | 787 | A | 987 | D | | |
| 188 | D | 388 | B | 588 | A | 788 | B | 988 | A | | |
| 189 | A | 389 | A | 589 | B | 789 | B | 989 | D | | |
| 190 | A | 390 | A | 590 | B | 790 | A | 990 | A | | |
| 191 | B | 391 | A | 591 | A | 791 | C | 991 | A | | |
| 192 | A | 392 | A | 592 | D | 792 | A | 992 | B | | |
| 193 | B | 393 | B | 593 | D | 793 | B | 993 | C | | |
| 194 | A | 394 | B | 594 | D | 794 | A | 994 | A | | |
| 195 | B | 395 | A | 595 | A | 795 | A | 995 | A | | |
| 196 | D | 396 | B | 596 | A | 796 | A | 996 | B | | |
| 197 | B | 397 | B | 597 | D | 797 | A | 997 | A | | |
| 198 | B | 398 | B | 598 | B | 798 | B | 998 | B | | |
| 199 | A | 399 | B | 599 | B | 799 | A | 999 | B | | |
| 200 | B | 400 | B | 600 | C | 800 | A | 1000 | A | | |
| 201 | B | 401 | B | 601 | A | 801 | C | 1001 | A | | |
| 202 | B | 402 | D | 602 | B | 802 | B | 1002 | A | | |
| 203 | B | 403 | B | 603 | B | 803 | A | 1003 | B | | |
| 204 | B | 404 | A | 604 | D | 804 | B | 1004 | B | | |
| 205 | A | 405 | B | 605 | D | 805 | B | 1005 | B | | |
| 206 | A | 406 | B | 606 | C | 806 | B | 1006 | B | | |
| 207 | A | 407 | B | 607 | B | 807 | A | 1007 | A | | |
| 208 | B | 408 | B | 608 | C | 808 | B | 1008 | B | | |
| 209 | A | 409 | B | 609 | B | 809 | A | 1009 | B | | |
| 210 | A | 410 | C | 610 | A | 810 | B | 1010 | C | | |
| 211 | B | 411 | B | 611 | D | 811 | A | 1011 | B | | |
| 212 | B | 412 | B | 612 | C | 812 | A | 1012 | B | | |
| 213 | B | 413 | A | 613 | A | 813 | A | 1013 | A | | |
| 214 | B | 414 | B | 614 | B | 814 | A | 1014 | A | | |
| 215 | A | 415 | B | 615 | B | 815 | B | 1015 | B | | |
| 216 | B | 416 | B | 616 | B | 816 | B | 1016 | A | | |
| 217 | B | 417 | A | 617 | B | 817 | A | 1017 | B | | |
| 218 | B | 418 | A | 618 | B | 818 | A | 1018 | B | | |
| 219 | B | 419 | B | 619 | B | 819 | A | 1019 | A | | |
| 220 | A | 420 | B | 620 | B | 820 | A | 1020 | A | | |
| 221 | B | 421 | A | 621 | B | 821 | A | 1021 | B | | |
| 222 | B | 422 | A | 622 | B | 822 | D | 1022 | D | | |
| 223 | A | 423 | B | 623 | A | 823 | B | 1023 | A | | |
| 224 | B | 424 | A | 624 | A | 824 | B | 1024 | C | | |
| 225 | A | 425 | C | 625 | A | 825 | B | 1025 | A | | |
| 226 | D | 426 | B | 626 | A | 826 | D | 1026 | C | | |
| 227 | B | 427 | B | 627 | A | 827 | B | 1027 | B | | |
| 228 | D | 428 | A | 628 | A | 828 | B | 1028 | B | | |
| 229 | B | 429 | D | 629 | B | 829 | A | 1029 | A | | |
| 230 | B | 430 | A | 630 | B | 830 | A | 1030 | A | | |
| 231 | B | 431 | B | 631 | B | 831 | B | 1031 | A | | |
| 232 | B | 432 | A | 632 | A | 832 | A | 1032 | A | | |
| 233 | A | 433 | B | 633 | A | 833 | A | 1033 | A | | |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

For reasons of completeness, various aspects of the present disclosure are set out in the following numbered clauses:

Clause 1. A compound of Formula (I):

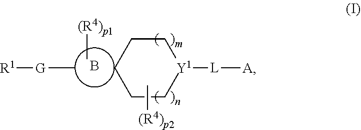

or a pharmaceutically acceptable salt thereof, wherein:
R¹ is substituted or unsubstituted heteroaryl, substituted or unsubstituted aryl, or a nitrogen protecting group;
G is a bond, —O—, —OC(R⁵)(R⁶)—, or —C(R⁵)(R⁶)—;
B is cycloalkyl or heterocyclyl comprising at least one nitrogen atom in its ring;
each occurrence of R⁴ is independently halogen, substituted or unsubstituted alkyl, substituted or unsubstituted alkoxy, or two instances of R⁴ on the same carbon form with that carbon a carbonyl;
Y¹ is N or CR⁵;
each occurrence of R⁵ and R⁶ are each independently hydrogen, halogen, or substituted or unsubstituted alkyl;
L is a bond, —SO₂—, —C(═O)—, —C(═O)C(R⁵)(R⁶)—, or —NHC(═O)—;
A is

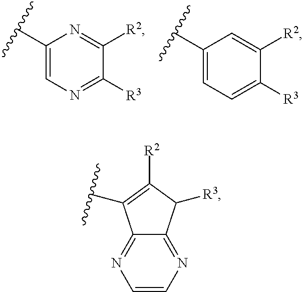

or substituted or unsubstituted 5-membered heteroaryl;
R² and R³ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
m and n are each independently 0, 1, or 2; and
p1 and p2 are each independently 0, 1, 2, 3, 4, 5, or 6.

Clause 2. The compound of clause 1, or a pharmaceutically acceptable salt thereof, wherein: R¹ is substituted or unsubstituted heteroaryl, or a nitrogen protecting group.

Clause 3. The compound of clause 1 or 2, or a pharmaceutically acceptable salt thereof, wherein: R¹ is substituted or unsubstituted heteroaryl.

Clause 4. The compound of any of clauses 1-3, or a pharmaceutically acceptable salt thereof, wherein: R¹ is substituted or unsubstituted pyridinyl.

Clause 5. The compound of any of clauses 1-4, or a pharmaceutically acceptable salt thereof, wherein: R¹ is substituted pyridinyl.

Clause 6. The compound of any of clauses 1-5, or a pharmaceutically acceptable salt thereof, wherein: R¹ is pyridinyl substituted with halogen or haloalkyl.

Clause 7. The compound of any of clauses 1-6, or a pharmaceutically acceptable salt thereof, wherein: R¹ is pyridinyl substituted with haloalkyl.

Clause 8. The compound of any of clauses 1-7, or a pharmaceutically acceptable salt thereof, wherein: R¹ is pyridinyl substituted with trifluoromethyl.

Clause 9. The compound of clause 1, or a pharmaceutically acceptable salt thereof, wherein: R¹ is substituted or unsubstituted phenyl.

Clause 10. The compound of any of clauses 1 or 9, or a pharmaceutically acceptable salt thereof, wherein: R¹ is unsubstituted phenyl or phenyl substituted with halogen or haloalkyl.

Clause 11. The compound of any of clauses 1-10, or a pharmaceutically acceptable salt thereof, wherein: R is

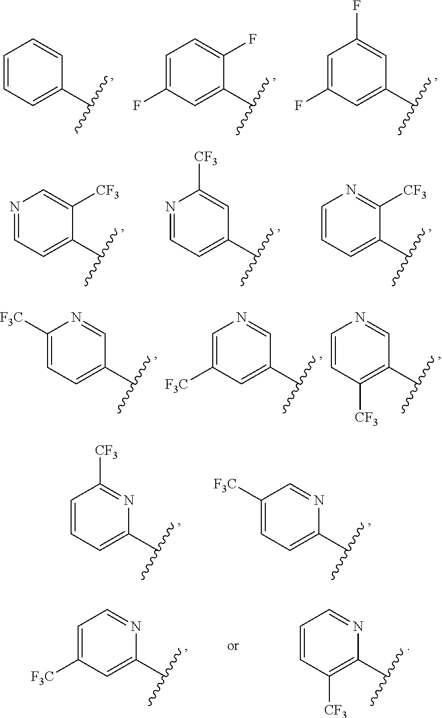

Clause 12. The compound of any of clauses 1-11, or a pharmaceutically acceptable salt thereof, wherein: R¹ is

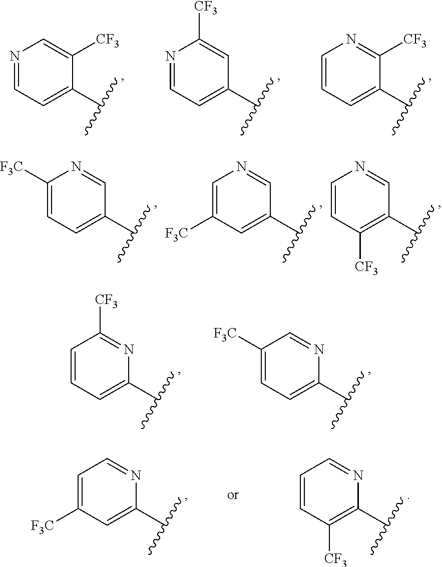

Clause 13. The compound of any of clauses 1 or 2, or a pharmaceutically acceptable salt thereof, wherein: R¹ is a nitrogen protecting group (e.g.,

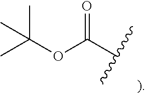

Clause 14. The compound of any of clauses 1-13, or a pharmaceutically acceptable salt thereof, wherein: A is

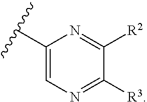

Clause 15. The compound of any of clauses 1-13, or a pharmaceutically acceptable salt thereof, wherein: A is

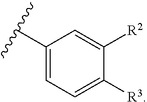

Clause 16. The compound of any of clauses 1-15, or a pharmaceutically acceptable salt thereof, wherein: R² and R³ are each independently hydrogen or substituted or unsubstituted heteroaryl; or R² and R³ together with the atoms to which they are attached form a substituted or unsubstituted aryl, or substituted or unsubstituted heteroaryl.

Clause 17. The compound of any of clauses 1-16, or a pharmaceutically acceptable salt thereof, wherein: R² is substituted or unsubstituted heteroaryl.

Clause 18. The compound of any of clauses 1-17, or a pharmaceutically acceptable salt thereof, wherein: R² is substituted or unsubstituted thiadiazolyl.

Clause 19. The compound of any of clauses 1-18, or a pharmaceutically acceptable salt thereof, wherein: R³ is hydrogen.

Clause 20. The compound of any of clauses 1-16, or a pharmaceutically acceptable salt thereof, wherein: R² and R³ together with the atoms to which they are attached form a substituted or unsubstituted heteroaryl.

Clause 21. The compound of any of clauses 1-20, or a pharmaceutically acceptable salt thereof, wherein: R² and R³ together with the atoms to which they are attached form a substituted or unsubstituted pyrrolyl or a substituted or unsubstituted pyrazolyl.

Clause 22. The compound of any of clauses 1-20, or a pharmaceutically acceptable salt thereof, wherein: R² and R³ together with the atoms to which they are attached form a substituted or unsubstituted pyrazolyl.

Clause 23. The compound of any of clauses 1-13, or a pharmaceutically acceptable salt thereof, wherein: A is substituted or unsubstituted 5-membered heteroaryl.

Clause 24. The compound of any of clauses 1-13, or a pharmaceutically acceptable salt thereof, wherein: A is substituted or unsubstituted pyrazolyl, or a substituted or unsubstituted thiazolyl.

Clause 25. The compound of any of clauses 1-22, or a pharmaceutically acceptable salt thereof, wherein: A is

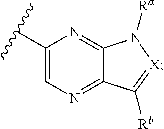

wherein X is N or CR^{a}; each R^{a} is independently hydrogen or substituted or unsubstituted alkyl; and R^{b} is hydrogen, halogen, substituted or unsubstituted alkyl, or substituted or unsubstituted alkoxy.

Clause 26. The compound of any of clauses 1-22, or a pharmaceutically acceptable salt thereof, wherein: A is

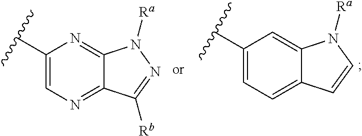

wherein R^{a} is hydrogen or substituted or unsubstituted alkyl.

Clause 27. The compound of any of clauses 1-22, or a pharmaceutically acceptable salt thereof, wherein: A is

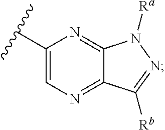

wherein R^{a} is substituted or unsubstituted alkyl.

Clause 28. The clause of any of clauses 1-22, or a pharmaceutically acceptable salt thereof, wherein: A is

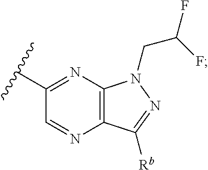

and R^{b} is hydrogen, halogen, substituted or unsubstituted alkyl, or substituted or unsubstituted alkoxy.

Clause 29. The clause of any of clauses 25-28, or a pharmaceutically acceptable salt thereof, wherein: R^{b} is halogen, substituted or unsubstituted alkyl, or substituted or unsubstituted alkoxy.

Clause 30. The compound of any of clauses 25-28, or a pharmaceutically acceptable salt thereof, wherein: R^{b} is hydrogen.

Clause 31. The compound of any of clauses 1-22, or a pharmaceutically acceptable salt thereof, wherein: A is

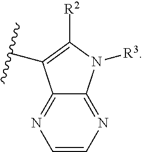

Clause 32. The compound of any of clauses 1-22, or a pharmaceutically acceptable salt thereof, wherein: A is

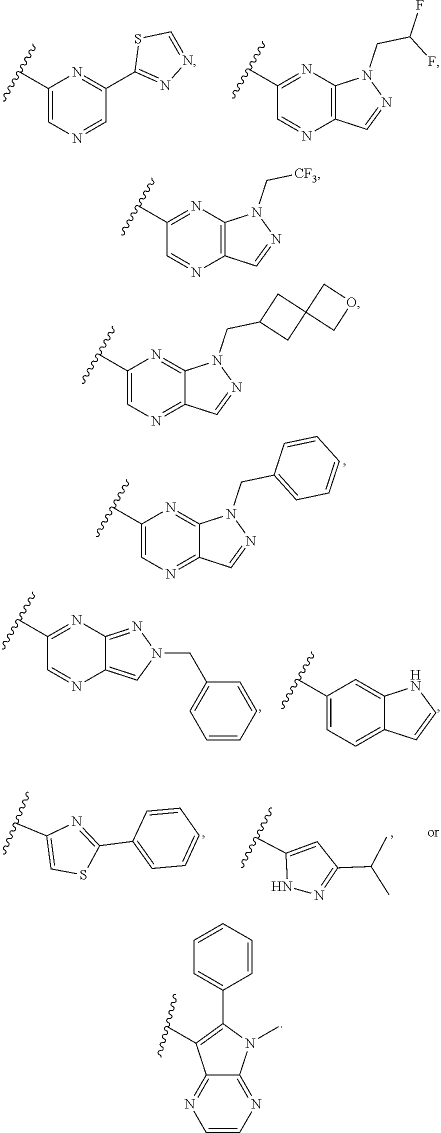

Clause 33. The compound of any of clauses 1-22, or a pharmaceutically acceptable salt thereof, wherein: A is

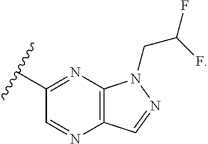 [SMILES: *c1cnc2cnn(CC(F)F)c2n1]

Clause 34. The compound of any of clauses 1-33, or a pharmaceutically acceptable salt thereof, wherein: G is a bond or —O—.

Clause 35. The compound of any of clauses 1-34, or a pharmaceutically acceptable salt thereof, wherein: G is a bond.

Clause 36. The compound of any of clauses 1-34 or a pharmaceutically acceptable salt thereof, wherein: G is —O—.

Clause 37. The compound of any of clauses 1-33, or a pharmaceutically acceptable salt thereof, wherein: G is —C(R⁵)(R⁶)—.

Clause 38. The compound of any of clauses 1-33, or a pharmaceutically acceptable salt thereof, wherein: G is —OC(R⁵)(R⁶)—.

Clause 39. The compound of any of clauses 1-38, or a pharmaceutically acceptable salt thereof, wherein: Y¹ is N.

Clause 40. The compound of any of clauses 1-38, or a pharmaceutically acceptable salt thereof, wherein: Y¹ is CR⁵.

Clause 41. The compound of any of clauses 1-40, or a pharmaceutically acceptable salt thereof, wherein: m is 1; and n is 1.

Clause 42. The compound of any of clauses 1-40, or a pharmaceutically acceptable salt thereof, wherein: m is 0; and n is 0.

Clause 43. The compound of any of clauses 1-40, or a pharmaceutically acceptable salt thereof, wherein: m is 2; and n is 0.

Clause 44. The compound of any of clauses 1-40, or a pharmaceutically acceptable salt thereof, wherein: m is 1; and n is 0.

Clause 45. The compound of any of clauses 1-40, or a pharmaceutically acceptable salt thereof, wherein: m is 0; and n is 1.

Clause 46. The compound of any of clauses 1-40, or a pharmaceutically acceptable salt thereof, wherein: m is 0; and n is 2.

Clause 47. The compound of any of clauses 1-46, or a pharmaceutically acceptable salt thereof, wherein: L is a bond.

Clause 48. The compound of any of clauses 1-46, or a pharmaceutically acceptable salt thereof, wherein: L is —C(═O)—.

Clause 49. The compound of any of clauses 1-46, or a pharmaceutically acceptable salt thereof, wherein: L is —C(═O)C(R⁵)(R⁶)—.

Clause 50. The compound of any of clauses 1-46, or a pharmaceutically acceptable salt thereof, wherein: L is —SO₂—.

Clause 51. The compound of any of clauses 1-46, or a pharmaceutically acceptable salt thereof, wherein: L is —NHC(═O)—.

Clause 52. The compound of any of clauses 1-51, or a pharmaceutically acceptable salt thereof, wherein: —B(R⁴)ₚ₁— is

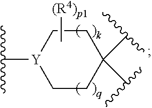

wherein: Y is N or CR⁵; k and q are each independently 0, 1, or 2; and Y is bound to G of Formula (I).

Clause 53. The compound of clause 52, or a pharmaceutically acceptable salt thereof, wherein: Y is N.

Clause 54. The compound of clause 52, or a pharmaceutically acceptable salt thereof, wherein: Y is CR⁵.

Clause 55. The compound of any of clauses 52-54, or a pharmaceutically acceptable salt thereof, wherein: k is 0; and q is 2.

Clause 56. The compound of any of clauses 52-54, or a pharmaceutically acceptable salt thereof, wherein: k is 1; and q is 1.

Clause 57. The compound of any of clauses 52-54, or a pharmaceutically acceptable salt thereof, wherein: k is 0; and q is 0.

Clause 58. The compound of any of clauses 52-54, or a pharmaceutically acceptable salt thereof, wherein: k is 2; and q is 0.

Clause 59. The compound of any of clauses 52-54, or a pharmaceutically acceptable salt thereof, wherein: k is 1; and q is 0.

Clause 60. The compound of any of clauses 52-54, or a pharmaceutically acceptable salt thereof, wherein: k is 0; and q is 1.

Clause 61. The compound of any of clauses 1-60, or a pharmaceutically acceptable salt thereof, wherein: —B(R⁴)ₚ₁— is

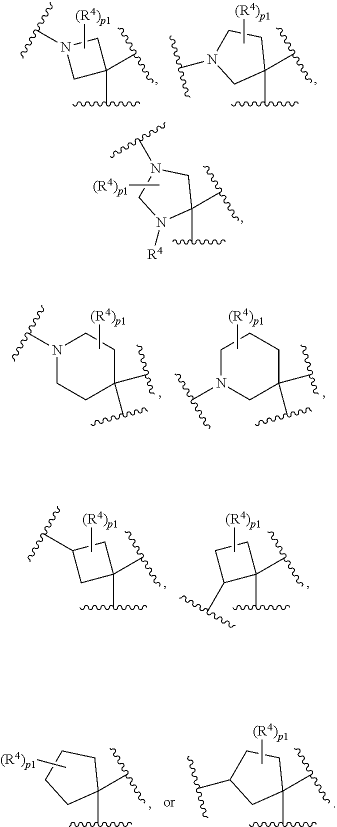

Clause 62. The compound of any of clauses 1-61, or a pharmaceutically acceptable salt thereof, wherein: —B(R⁴)ₚ₁— is

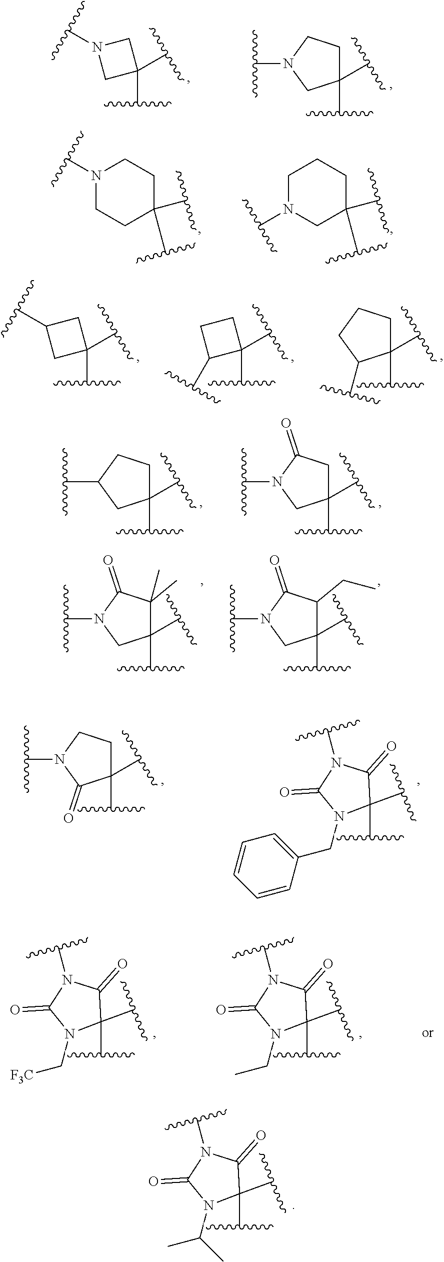

Clause 63. The compound of clause 1, wherein the compound is of formula (I-a):

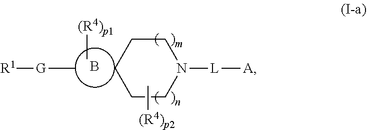

or a pharmaceutically acceptable salt thereof.

Clause 64. The compound of clause 1 or 52, wherein the compound is of formula (I-b):

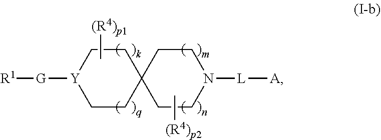

or a pharmaceutically acceptable salt thereof, wherein: Y is N or CR⁵; and k and q are each independently 0, 1, or 2.

Clause 65. The compound of clause 64, or a pharmaceutically acceptable salt thereof, wherein: Y is N.

Clause 66. The compound of clause 64, or a pharmaceutically acceptable salt thereof, wherein: Y is CR⁵.

Clause 67. The compound of any of clauses 64-66, or a pharmaceutically acceptable salt thereof, wherein: k is 0; and q is 2.

Clause 68. The compound of any of clauses 64-66, or a pharmaceutically acceptable salt thereof, wherein: k is 1; and q is 1.

Clause 69. The compound of any of clauses 64-66, or a pharmaceutically acceptable salt thereof, wherein: k is 0; and q is 0.

Clause 70. The compound of any of clauses 64-66, or a pharmaceutically acceptable salt thereof, wherein: k is 2; and q is 0.

Clause 71. The compound of any of clauses 64-66, or a pharmaceutically acceptable salt thereof, wherein: k is 1; and q is 0.

Clause 72. The compound of any of clauses 64-66, or a pharmaceutically acceptable salt thereof, wherein: k is 0; and q is 1.

Clause 73. The compound of clause 1, wherein the compound is of formula (I-c):

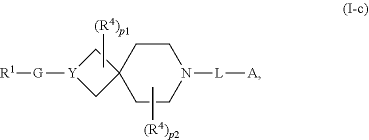

or a pharmaceutically acceptable salt thereof.

Clause 74. The compound of clause 1, wherein the compound is of formula (I-c-1):

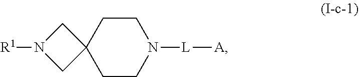

or a pharmaceutically acceptable salt thereof.

Clause 75. The compound of clause 1, wherein the compound is of formula (I-d):

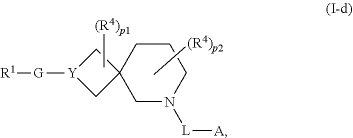

or a pharmaceutically acceptable salt thereof.

Clause 76. The compound of clause 1, wherein the compound is of formula (I-d-1):

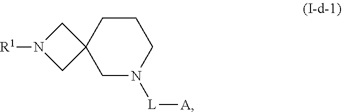

or a pharmaceutically acceptable salt thereof.

Clause 77. The compound of clause 1, wherein the compound is of formula (I-d-4):

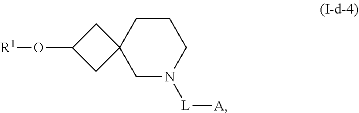

or a pharmaceutically acceptable salt thereof.

Clause 78. The compound of clause 1, wherein the compound is of formula (I-e):

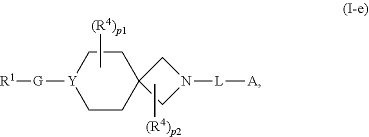

or a pharmaceutically acceptable salt thereof.

Clause 79. The compound of clause 1, wherein the compound is of formula (I-e-1):

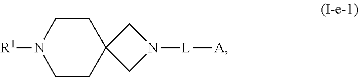

or a pharmaceutically acceptable salt thereof.

Clause 80. The compound of clause 1, wherein the compound is of formula (I-f):

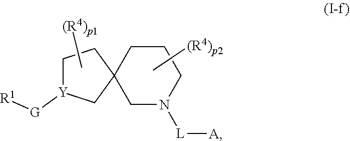

or a pharmaceutically acceptable salt thereof.

Clause 81. The compound of clause 1, wherein the compound is of formula (I-f-1):

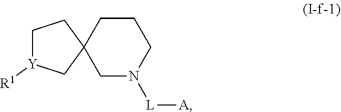

or a pharmaceutically acceptable salt thereof.

Clause 82. The compound of clause 1, wherein the compound is of formula (I-g):

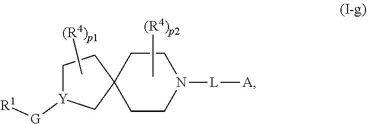

or a pharmaceutically acceptable salt thereof.

Clause 83. The compound of clause 1, wherein the compound is of formula (I-g-1):

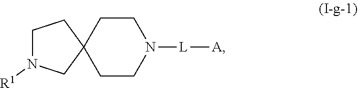

or a pharmaceutically acceptable salt thereof.

Clause 84. The compound of clause 1, wherein the compound is of formula (I-h):

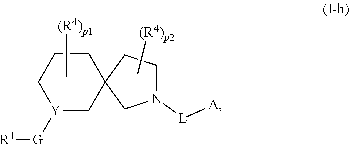

or a pharmaceutically acceptable salt thereof.

Clause 85. The compound of clause 1, wherein the compound is of formula (I-h-1):

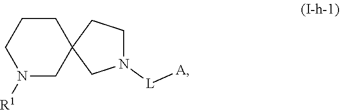

or a pharmaceutically acceptable salt thereof.

Clause 86. The compound of clause 1, wherein the compound is of formula (I-i):

or a pharmaceutically acceptable salt thereof.

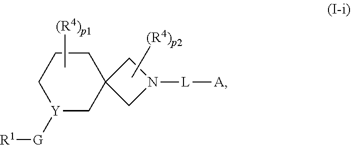

Clause 87. The compound of clause 1, wherein the compound is of formula (I-i-1):

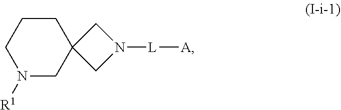

or a pharmaceutically acceptable salt thereof.

Clause 88. The compound of clause 1, wherein the compound is of formula (I-j):

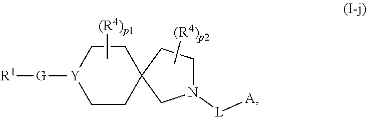

or a pharmaceutically acceptable salt thereof.

Clause 89. The compound of clause 1, wherein the compound is of formula (I-j-1):

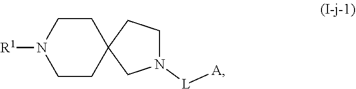

or a pharmaceutically acceptable salt thereof.

Clause 90. The compound of clause 1, wherein the compound is of formula (I-k):

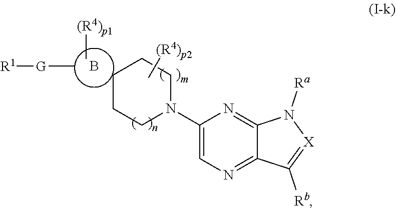

or a pharmaceutically acceptable salt thereof; wherein: X is N or CR^{a}; each R^{a} is independently hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

Clause 91. The compound of clause 1, wherein the compound is of formula (I-l):

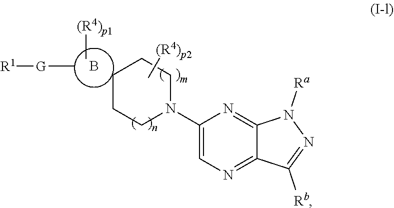

or a pharmaceutically acceptable salt thereof; wherein: R^{a} is independently hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

Clause 92. The compound of clause 1, wherein the compound is of formula (I-m):

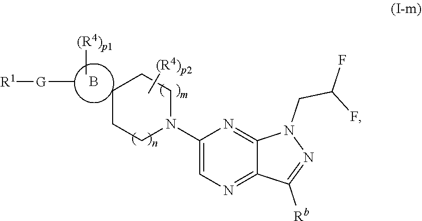

or a pharmaceutically acceptable salt thereof; and R is hydrogen, halogen, substituted or unsubstituted alkyl, or substituted or unsubstituted alkoxy.

Clause 93. The compound of clause 52 or 64, wherein the compound is of formula (I-n):

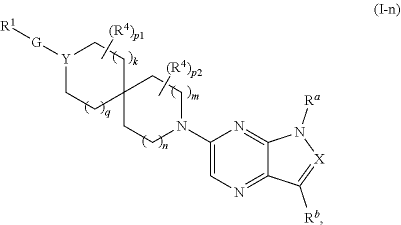

or a pharmaceutically acceptable salt thereof; wherein: X is N or CR^{a}; each R^{a} is independently hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

Clause 94. The compound of clause 52 or 64, wherein the compound is of formula (I-o):

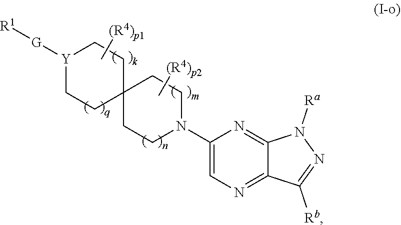

or a pharmaceutically acceptable salt thereof, wherein: R^{a} is hydrogen, substituted or unsubstituted alkyl, or substituted or unsubstituted heterocyclyl; and R^{b} is hydrogen, halogen, substituted or unsubstituted alkyl, or substituted or unsubstituted alkoxy.

Clause 95. The compound of clause 52 or 64, wherein the compound is of formula (I-p):

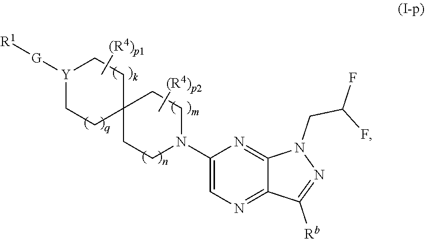

or a pharmaceutically acceptable salt thereof; wherein R^{b} is hydrogen, halogen, substituted or unsubstituted alkyl, or substituted or unsubstituted alkoxy.

Clause 96. The compound of clause 52 or 64, wherein the compound is of formula (I-q):

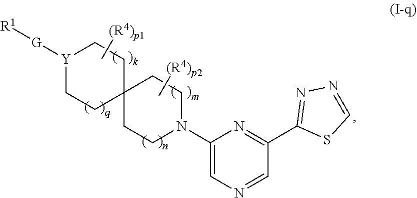

or a pharmaceutically acceptable salt thereof.

Clause 97. The compound of clause 52 or 64, wherein the compound is of formula (I-r):

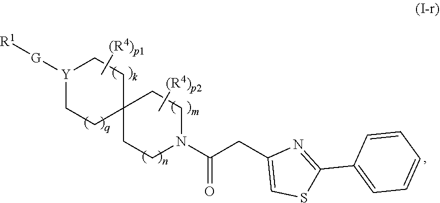

or a pharmaceutically acceptable salt thereof.

Clause 98. The compound of clause 1, wherein the compound is of formula (I-s):

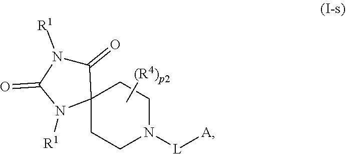

or a pharmaceutically acceptable salt thereof.

Clause 99. The compound of clause 1, wherein the compound is:

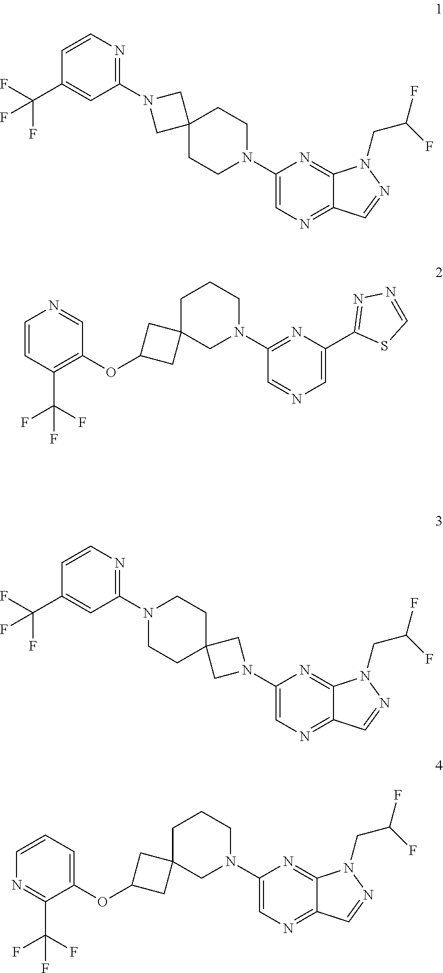 [SMILES: FC(F)Cn1ncc2ncc(N3CCC4(CC3)CN(c3cc(C(F)(F)F)ccn3)C4)nc21 ; FC(F)(F)c1ccncc1OC1CC2(CCCN(c3cncc(-c4nncs4)n3)C2)C1 ; FC(F)Cn1ncc2ncc(N3CC4(CCN(c5cc(C(F)(F)F)ccn5)CC4)C3)nc21 ; FC(F)Cn1ncc2ncc(N3CCCC4(CC(Oc5cccnc5C(F)(F)F)C4)C3)nc21]

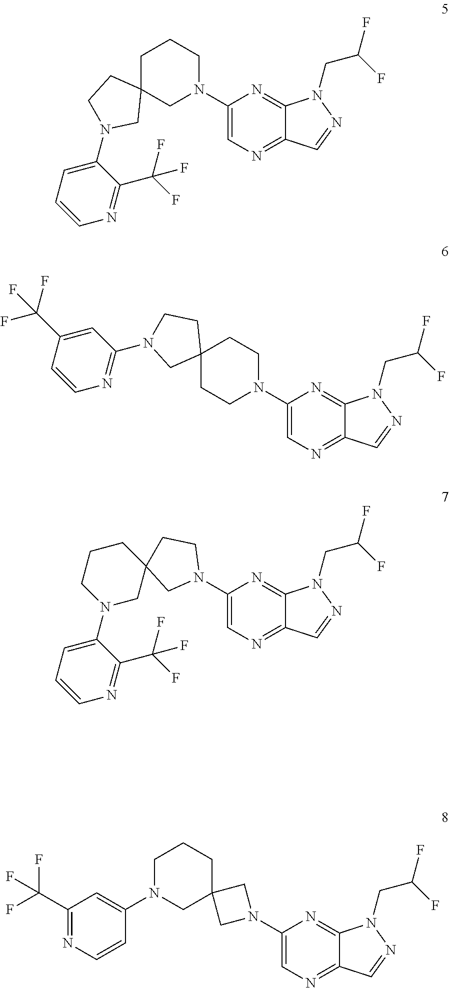 [SMILES: FC(F)Cn1ncc2ncc(N3CCCC4(CCN(c5cccnc5C(F)(F)F)C4)C3)nc21 ; FC(F)Cn1ncc2ncc(N3CCC4(CCN(c5cc(C(F)(F)F)ccn5)C4)CC3)nc21 ; FC(F)Cn1ncc2ncc(N3CCC4(CCCN(c5cccnc5C(F)(F)F)C4)C3)nc21 ; FC(F)Cn1ncc2ncc(N3CC4(CCCN(c5ccnc(C(F)(F)F)c5)C4)C3)nc21]

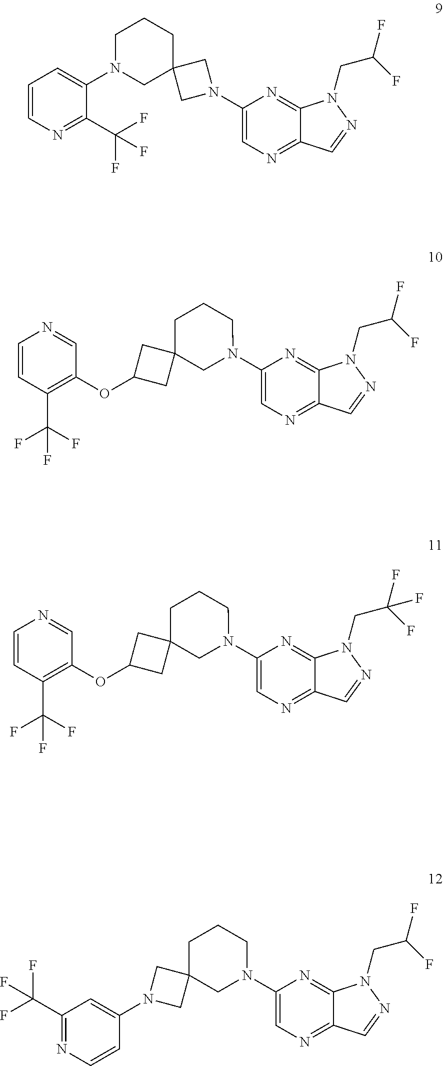 [SMILES: FC(F)Cn1ncc2ncc(N3CC4(CCCN(c5cccnc5C(F)(F)F)C4)C3)nc21 ; FC(F)Cn1ncc2ncc(N3CCCC4(CC(Oc5cnccc5C(F)(F)F)C4)C3)nc21 ; FC(F)(F)Cn1ncc2ncc(N3CCCC4(CC(Oc5cnccc5C(F)(F)F)C4)C3)nc21 ; FC(F)Cn1ncc2ncc(N3CCCC4(CN(c5ccnc(C(F)(F)F)c5)C4)C3)nc21]

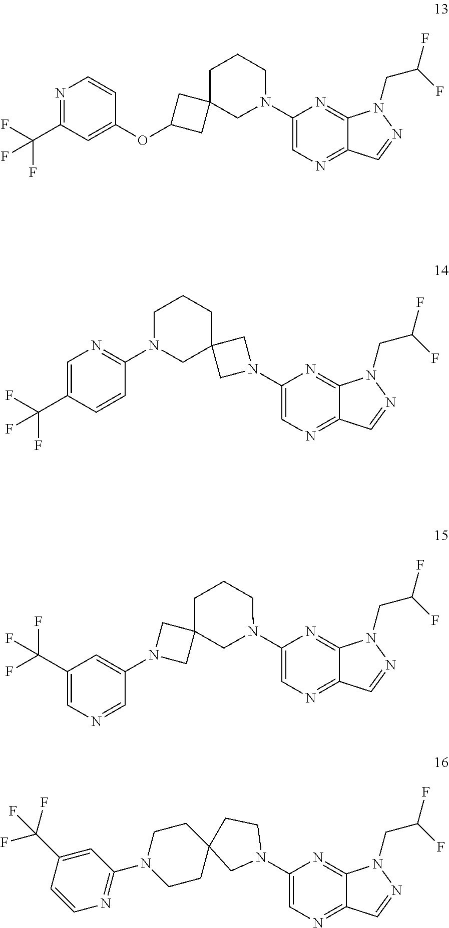 [SMILES: FC(F)Cn1ncc2ncc(N3CCCC4(CC(Oc5ccnc(C(F)(F)F)c5)C4)C3)nc21 ; FC(F)Cn1ncc2ncc(N3CC4(CCCN(c5ccc(C(F)(F)F)cn5)C4)C3)nc21 ; FC(F)Cn1ncc2ncc(N3CCCC4(CN(c5cncc(C(F)(F)F)c5)C4)C3)nc21 ; FC(F)Cn1ncc2ncc(N3CCC4(CCN(c5cc(C(F)(F)F)ccn5)CC4)C3)nc21]

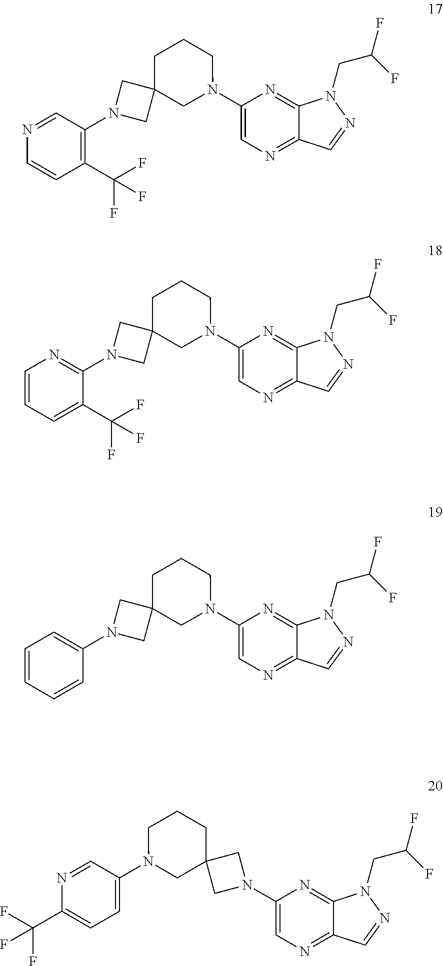 [SMILES: FC(F)Cn1ncc2ncc(N3CCCC4(C3)CN(c3cnccc3C(F)(F)F)C4)nc21 ; FC(F)Cn1ncc2ncc(N3CCCC4(C3)CN(c3ncccc3C(F)(F)F)C4)nc21 ; FC(F)Cn1ncc2ncc(N3CCCC4(CN(c5ccccc5)C4)C3)nc21 ; FC(F)Cn1ncc2ncc(N3CC4(CCCN(c5ccc(C(F)(F)F)nc5)C4)C3)nc21]

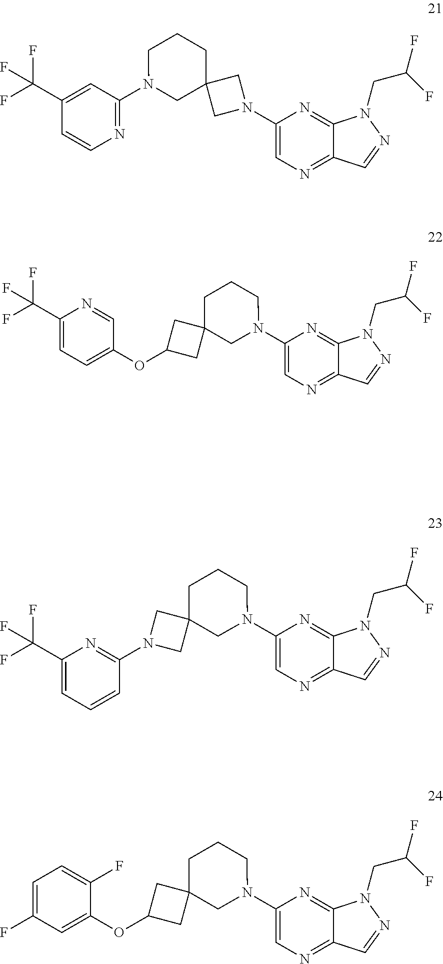 [SMILES: FC(F)Cn1ncc2ncc(N3CC4(CCCN(c5cc(C(F)(F)F)ccn5)C4)C3)nc21 ; FC(F)Cn1ncc2ncc(N3CCCC4(CC(Oc5ccc(C(F)(F)F)nc5)C4)C3)nc21 ; FC(F)Cn1ncc2ncc(N3CCCC4(CN(c5cccc(C(F)(F)F)n5)C4)C3)nc21 ; Fc1ccc(F)c(OC2CC3(CCCN(c4cnc5cnn(CC(F)F)c5n4)C3)C2)c1]

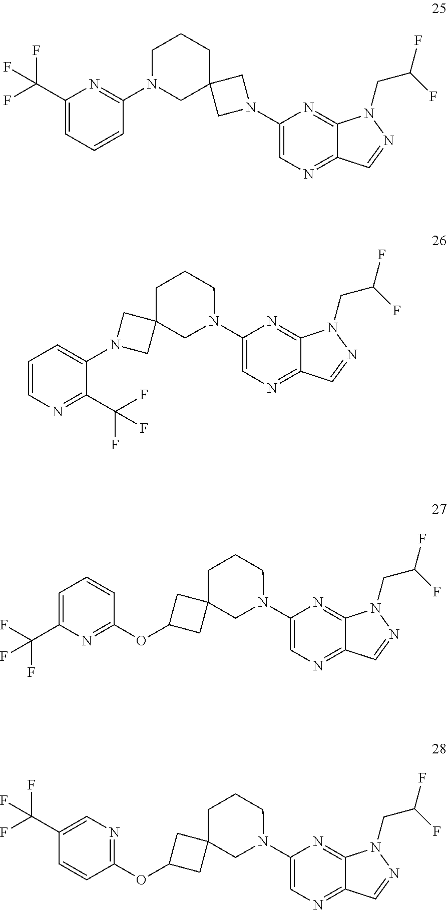 [SMILES: FC(F)Cn1ncc2ncc(N3CC4(CCCN(c5cccc(C(F)(F)F)n5)C4)C3)nc21 ; FC(F)Cn1ncc2ncc(N3CCCC4(C3)CN(c3cccnc3C(F)(F)F)C4)nc21 ; FC(F)Cn1ncc2ncc(N3CCCC4(CC(Oc5cccc(C(F)(F)F)n5)C4)C3)nc21 ; FC(F)Cn1ncc2ncc(N3CCCC4(CC(Oc5ccc(C(F)(F)F)cn5)C4)C3)nc21]

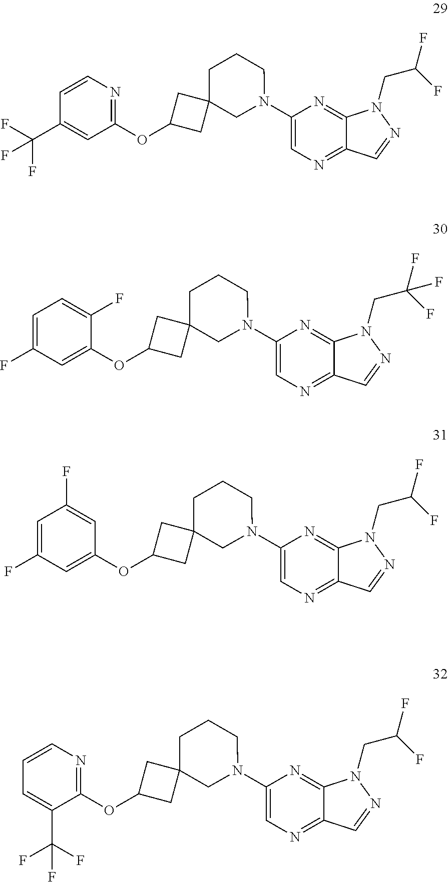 [SMILES: FC(F)Cn1ncc2ncc(N3CCCC4(CC(Oc5cc(C(F)(F)F)ccn5)C4)C3)nc21 ; Fc1ccc(F)c(OC2CC3(CCCN(c4cnc5cnn(CC(F)(F)F)c5n4)C3)C2)c1 ; Fc1cc(F)cc(OC2CC3(CCCN(c4cnc5cnn(CC(F)F)c5n4)C3)C2)c1 ; FC(F)Cn1ncc2ncc(N3CCCC4(CC(Oc5ncccc5C(F)(F)F)C4)C3)nc21]

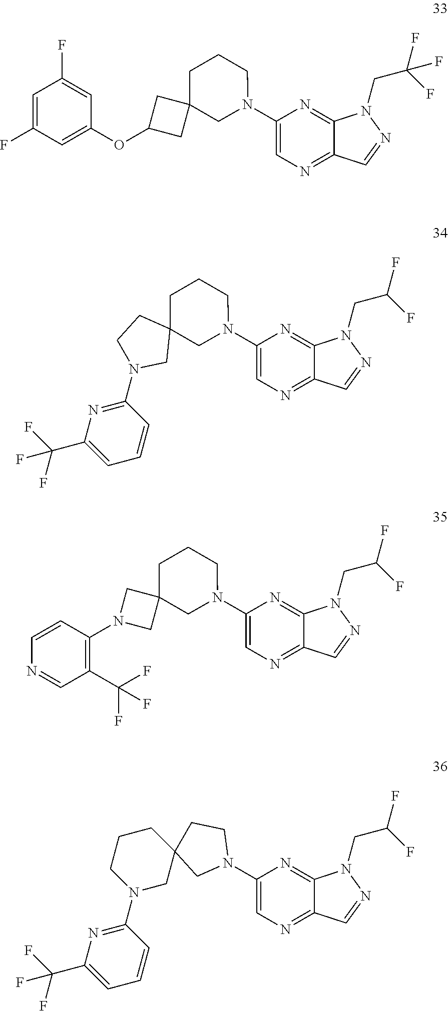 [SMILES: Fc1cc(F)cc(OC2CC3(CCCN(c4cnc5cnn(CC(F)(F)F)c5n4)C3)C2)c1 ; FC(F)Cn1ncc2ncc(N3CCCC4(CCN(c5cccc(C(F)(F)F)n5)C4)C3)nc21 ; FC(F)Cn1ncc2ncc(N3CCCC4(C3)CN(c3ccncc3C(F)(F)F)C4)nc21 ; FC(F)Cn1ncc2ncc(N3CCC4(CCCN(c5cccc(C(F)(F)F)n5)C4)C3)nc21]

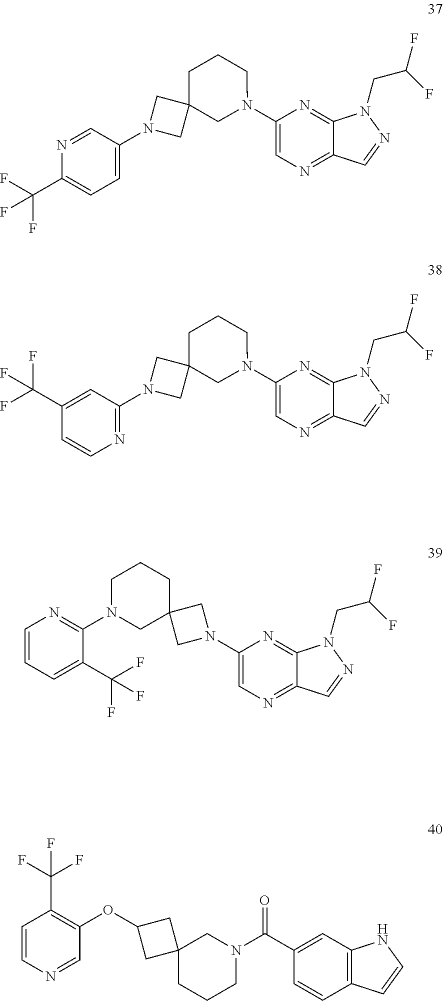 [SMILES: FC(F)Cn1ncc2ncc(N3CCCC4(CN(c5ccc(C(F)(F)F)nc5)C4)C3)nc21 ; FC(F)Cn1ncc2ncc(N3CCCC4(CN(c5cc(C(F)(F)F)ccn5)C4)C3)nc21 ; FC(F)Cn1ncc2ncc(N3CC4(CCCN(c5ncccc5C(F)(F)F)C4)C3)nc21 ; O=C(c1ccc2cc[nH]c2c1)N1CCCC2(CC(Oc3cnccc3C(F)(F)F)C2)C1]

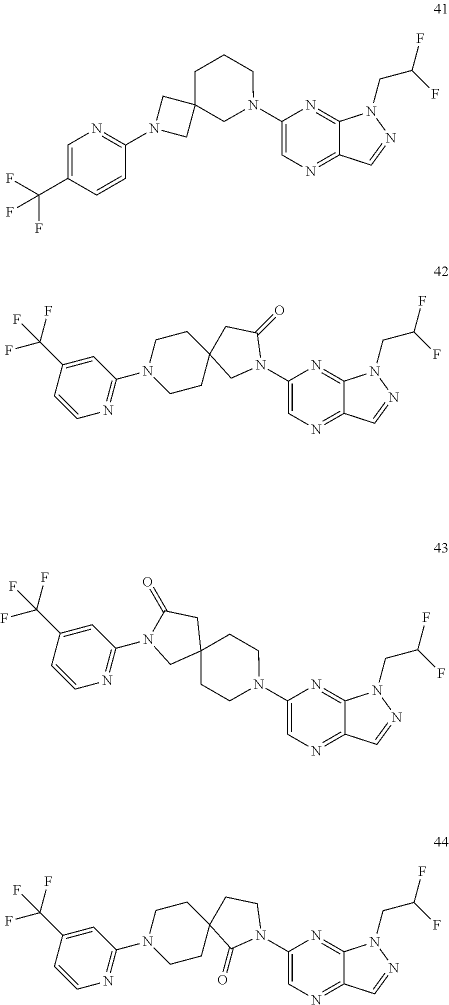 [SMILES: FC(F)Cn1ncc2ncc(N3CCCC4(CN(c5ccc(C(F)(F)F)cn5)C4)C3)nc21 ; O=C1CC2(CCN(c3cc(C(F)(F)F)ccn3)CC2)CN1c1cnc2cnn(CC(F)F)c2n1 ; O=C1CC2(CCN(c3cnc4cnn(CC(F)F)c4n3)CC2)CN1c1cc(C(F)(F)F)ccn1 ; O=C1N(c2cnc3cnn(CC(F)F)c3n2)CCC12CCN(c1cc(C(F)(F)F)ccn1)CC2]

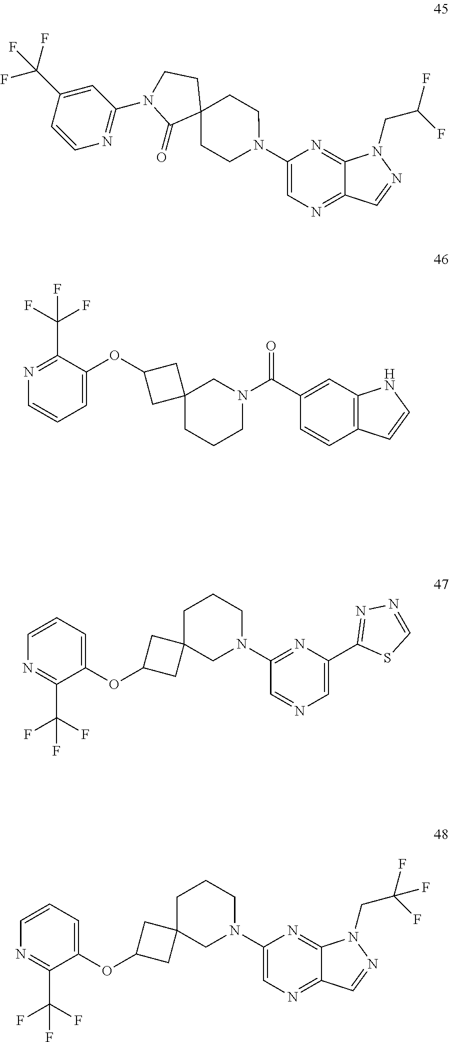 [SMILES: O=C1N(c2cc(C(F)(F)F)ccn2)CCC12CCN(c1cnc3cnn(CC(F)F)c3n1)CC2 ; O=C(c1ccc2cc[nH]c2c1)N1CCCC2(CC(Oc3cccnc3C(F)(F)F)C2)C1 ; FC(F)(F)c1ncccc1OC1CC2(CCCN(c3cncc(-c4nncs4)n3)C2)C1 ; FC(F)(F)Cn1ncc2ncc(N3CCCC4(CC(Oc5cccnc5C(F)(F)F)C4)C3)nc21]

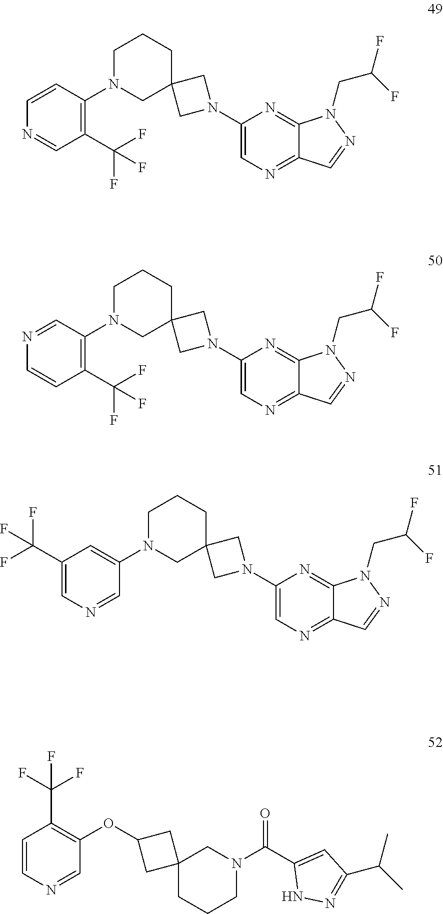 [SMILES: FC(F)Cn1ncc2ncc(N3CC4(CCCN(c5ccncc5C(F)(F)F)C4)C3)nc21 ; FC(F)Cn1ncc2ncc(N3CC4(CCCN(c5cnccc5C(F)(F)F)C4)C3)nc21 ; FC(F)Cn1ncc2ncc(N3CC4(CCCN(c5cncc(C(F)(F)F)c5)C4)C3)nc21 ; CC(C)c1cc(C(=O)N2CCCC3(CC(Oc4cnccc4C(F)(F)F)C3)C2)[nH]n1]

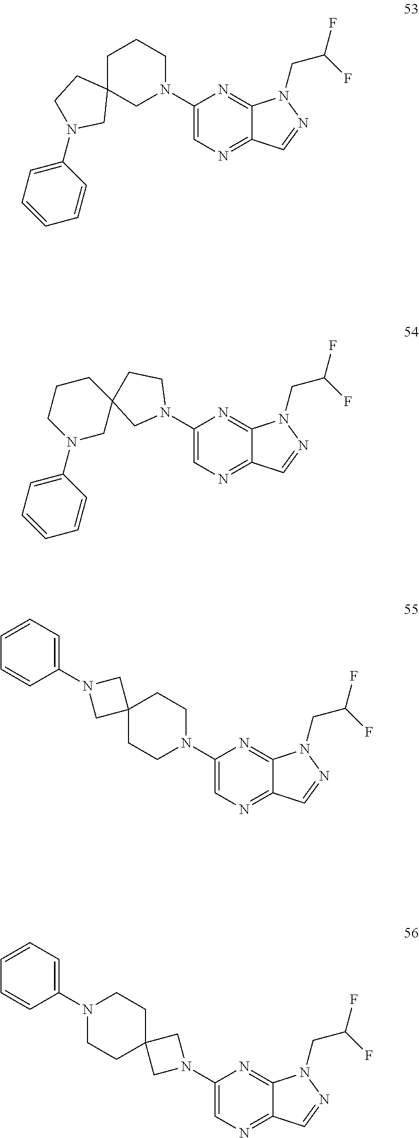 [SMILES: FC(F)Cn1ncc2ncc(N3CCCC4(CCN(c5ccccc5)C4)C3)nc21 ; FC(F)Cn1ncc2ncc(N3CCC4(CCCN(c5ccccc5)C4)C3)nc21 ; FC(F)Cn1ncc2ncc(N3CCC4(CC3)CN(c3ccccc3)C4)nc21 ; FC(F)Cn1ncc2ncc(N3CC4(CCN(c5ccccc5)CC4)C3)nc21]

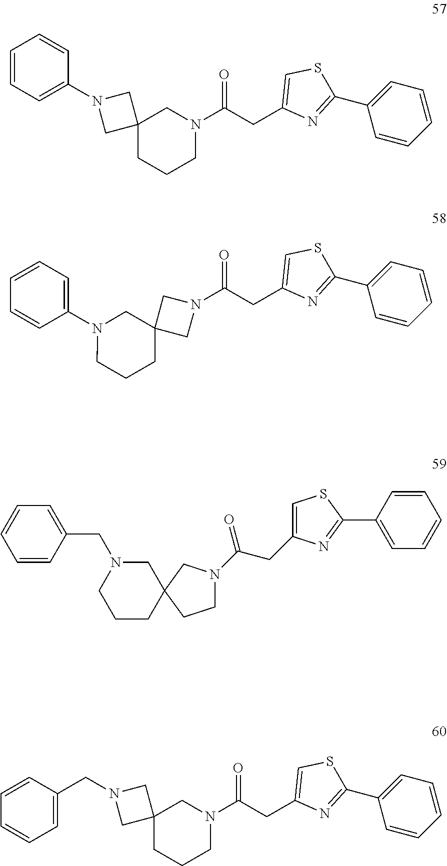 [SMILES: O=C(Cc1csc(-c2ccccc2)n1)N1CCCC2(C1)CN(c1ccccc1)C2 ; O=C(Cc1csc(-c2ccccc2)n1)N1CC2(CCCN(c3ccccc3)C2)C1 ; O=C(Cc1csc(-c2ccccc2)n1)N1CCC2(CCCN(Cc3ccccc3)C2)C1 ; O=C(Cc1csc(-c2ccccc2)n1)N1CCCC2(CN(Cc3ccccc3)C2)C1]

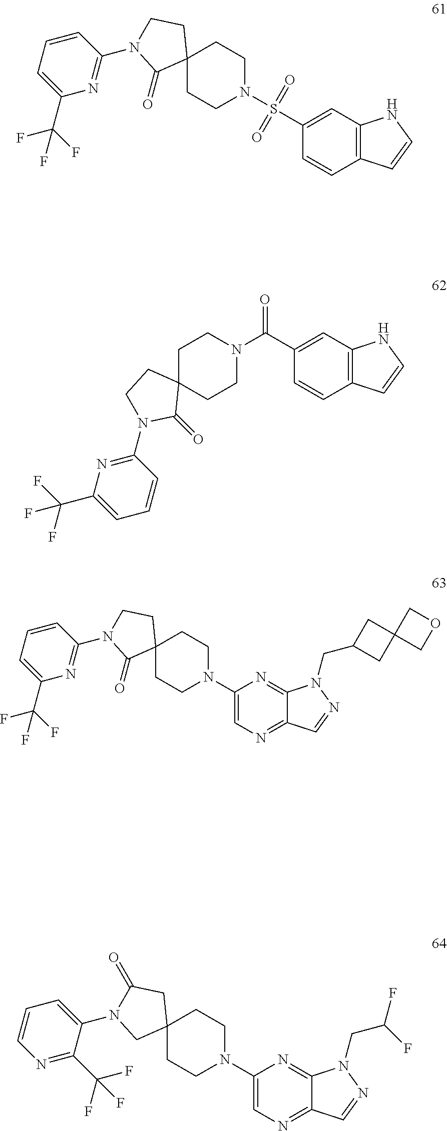 [SMILES: O=C1N(c2cccc(C(F)(F)F)n2)CCC12CCN(S(=O)(=O)c1ccc3cc[nH]c3c1)CC2 ; O=C(c1ccc2cc[nH]c2c1)N1CCC2(CC1)CCN(c1cccc(C(F)(F)F)n1)C2=O ; O=C1N(c2cccc(C(F)(F)F)n2)CCC12CCN(c1cnc3cnn(CC4CC5(COC5)C4)c3n1)CC2 ; O=C1CC2(CCN(c3cnc4cnn(CC(F)F)c4n3)CC2)CN1c1cccnc1C(F)(F)F]

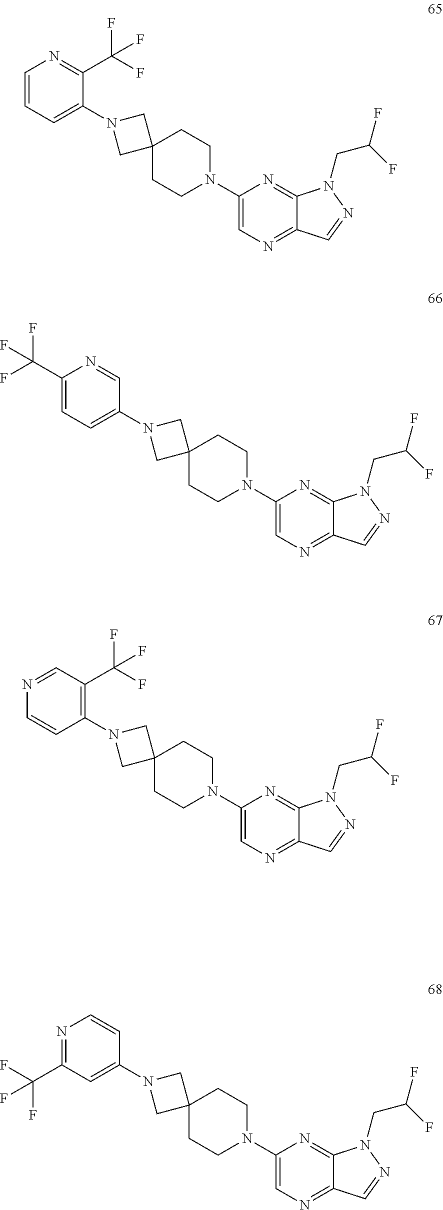 [SMILES: FC(F)Cn1ncc2ncc(N3CCC4(CC3)CN(c3cccnc3C(F)(F)F)C4)nc21 ; FC(F)Cn1ncc2ncc(N3CCC4(CC3)CN(c3ccc(C(F)(F)F)nc3)C4)nc21 ; FC(F)Cn1ncc2ncc(N3CCC4(CC3)CN(c3ccncc3C(F)(F)F)C4)nc21 ; FC(F)Cn1ncc2ncc(N3CCC4(CC3)CN(c3ccnc(C(F)(F)F)c3)C4)nc21]

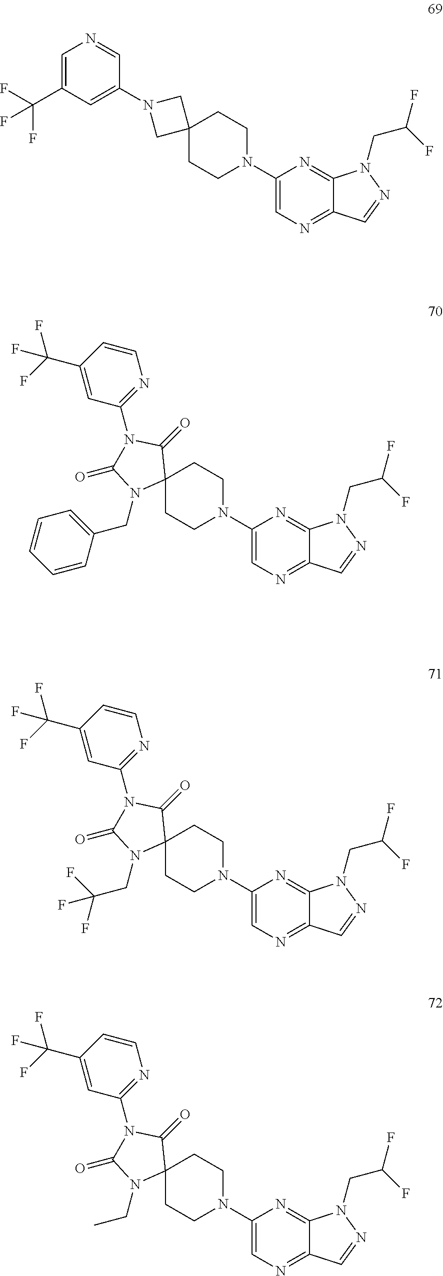 [SMILES: FC(F)Cn1ncc2ncc(N3CCC4(CC3)CN(c3cncc(C(F)(F)F)c3)C4)nc21 ; O=C1N(c2cc(C(F)(F)F)ccn2)C(=O)C2(CCN(c3cnc4cnn(CC(F)F)c4n3)CC2)N1Cc1ccccc1 ; O=C1N(c2cc(C(F)(F)F)ccn2)C(=O)C2(CCN(c3cnc4cnn(CC(F)F)c4n3)CC2)N1CC(F)(F)F ; CCN1C(=O)N(c2cc(C(F)(F)F)ccn2)C(=O)C12CCN(c1cnc3cnn(CC(F)F)c3n1)CC2]

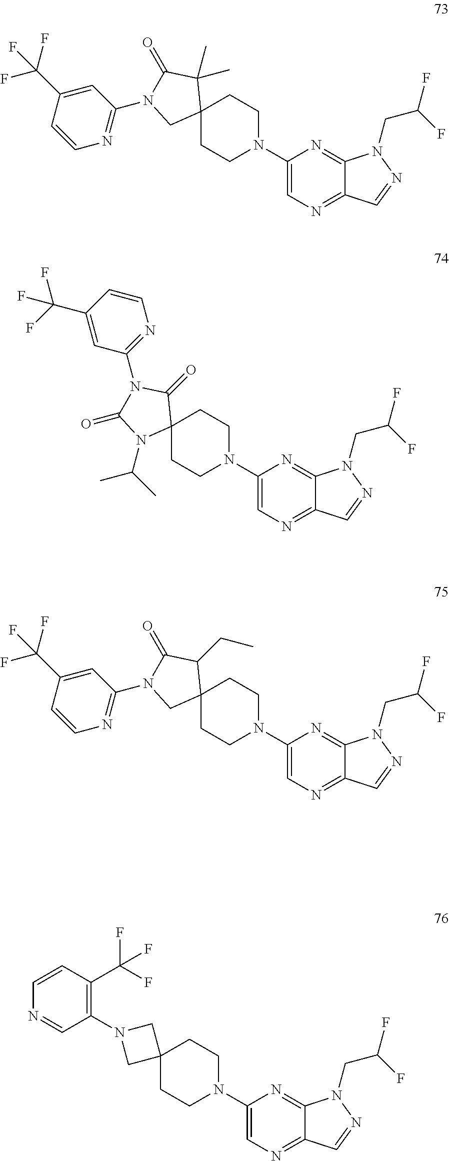 [SMILES: CC1(C)C(=O)N(c2cc(C(F)(F)F)ccn2)CC12CCN(c1cnc3cnn(CC(F)F)c3n1)CC2 ; CC(C)N1C(=O)N(c2cc(C(F)(F)F)ccn2)C(=O)C12CCN(c1cnc3cnn(CC(F)F)c3n1)CC2 ; CCC1C(=O)N(c2cc(C(F)(F)F)ccn2)CC12CCN(c1cnc3cnn(CC(F)F)c3n1)CC2 ; FC(F)Cn1ncc2ncc(N3CCC4(CC3)CN(c3cnccc3C(F)(F)F)C4)nc21]

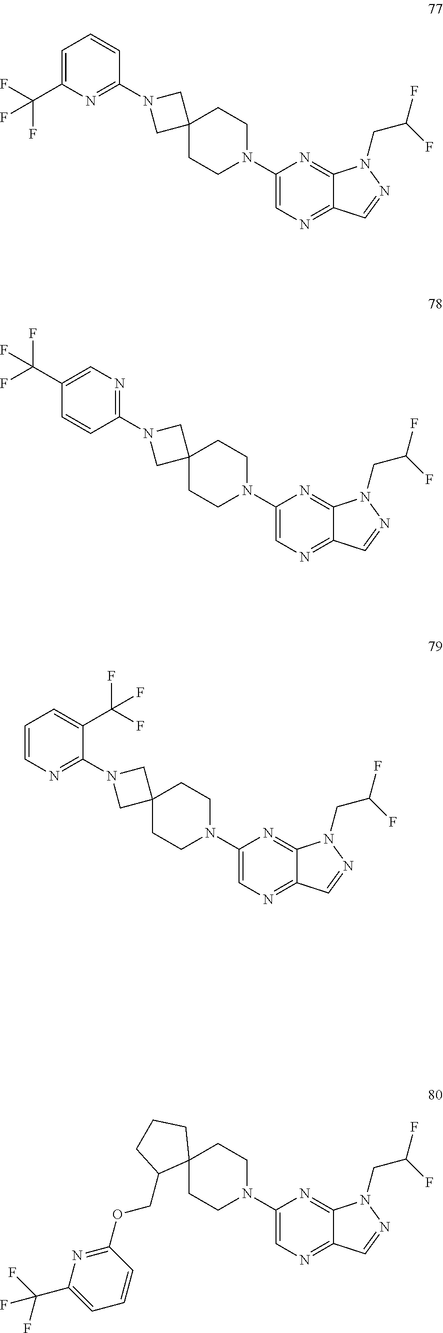 [SMILES: FC(F)Cn1ncc2ncc(N3CCC4(CC3)CN(c3cccc(C(F)(F)F)n3)C4)nc21 ; FC(F)Cn1ncc2ncc(N3CCC4(CC3)CN(c3ccc(C(F)(F)F)cn3)C4)nc21 ; FC(F)Cn1ncc2ncc(N3CCC4(CC3)CN(c3ncccc3C(F)(F)F)C4)nc21 ; FC(F)Cn1ncc2ncc(N3CCC4(CCCC4COc4cccc(C(F)(F)F)n4)CC3)nc21]

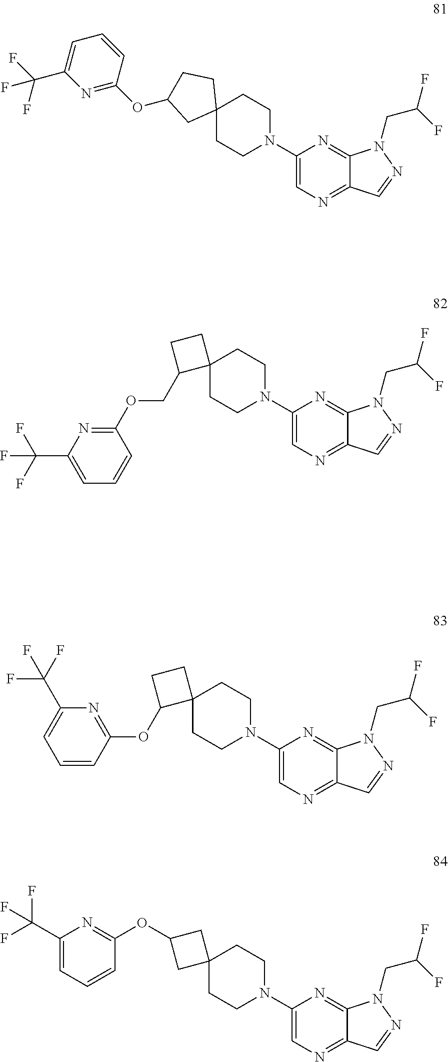 [SMILES: FC(F)Cn1ncc2ncc(N3CCC4(CCC(Oc5cccc(C(F)(F)F)n5)C4)CC3)nc21 ; FC(F)Cn1ncc2ncc(N3CCC4(CCC4COc4cccc(C(F)(F)F)n4)CC3)nc21 ; FC(F)Cn1ncc2ncc(N3CCC4(CCC4Oc4cccc(C(F)(F)F)n4)CC3)nc21 ; FC(F)Cn1ncc2ncc(N3CCC4(CC3)CC(Oc3cccc(C(F)(F)F)n3)C4)nc21]

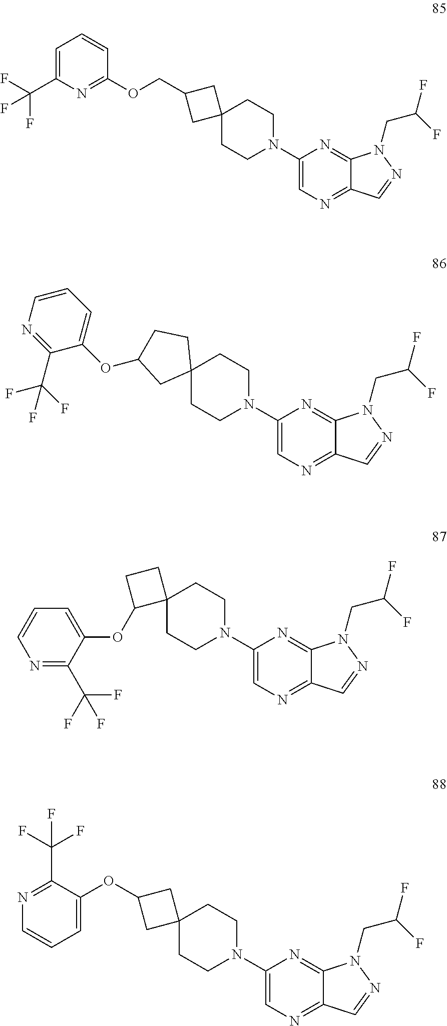 [SMILES: FC(F)Cn1ncc2ncc(N3CCC4(CC3)CC(COc3cccc(C(F)(F)F)n3)C4)nc21 ; FC(F)Cn1ncc2ncc(N3CCC4(CCC(Oc5cccnc5C(F)(F)F)C4)CC3)nc21 ; FC(F)Cn1ncc2ncc(N3CCC4(CCC4Oc4cccnc4C(F)(F)F)CC3)nc21 ; FC(F)Cn1ncc2ncc(N3CCC4(CC3)CC(Oc3cccnc3C(F)(F)F)C4)nc21]

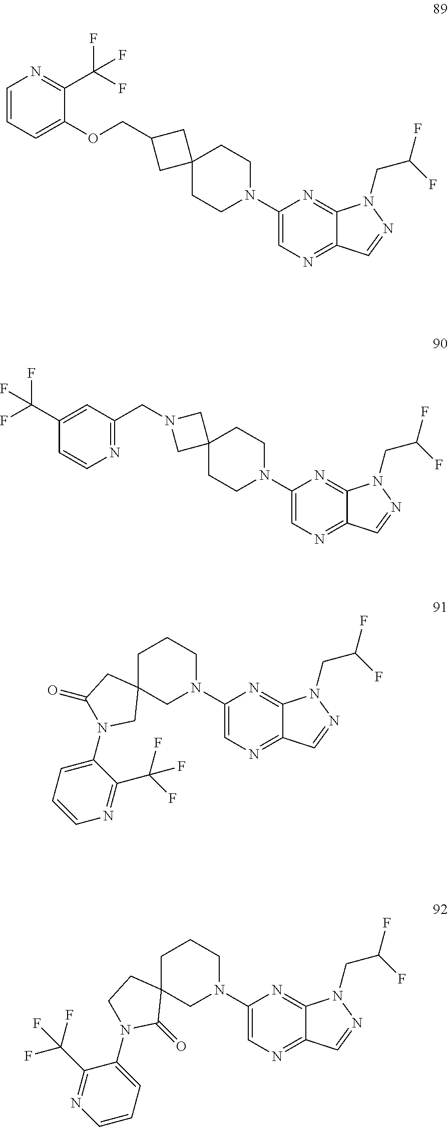 [SMILES: FC(F)Cn1ncc2ncc(N3CCC4(CC3)CC(COc3cccnc3C(F)(F)F)C4)nc21 ; FC(F)Cn1ncc2ncc(N3CCC4(CC3)CN(Cc3cc(C(F)(F)F)ccn3)C4)nc21 ; O=C1CC2(CCCN(c3cnc4cnn(CC(F)F)c4n3)C2)CN1c1cccnc1C(F)(F)F ; O=C1N(c2cccnc2C(F)(F)F)CCC12CCCN(c1cnc3cnn(CC(F)F)c3n1)C2]

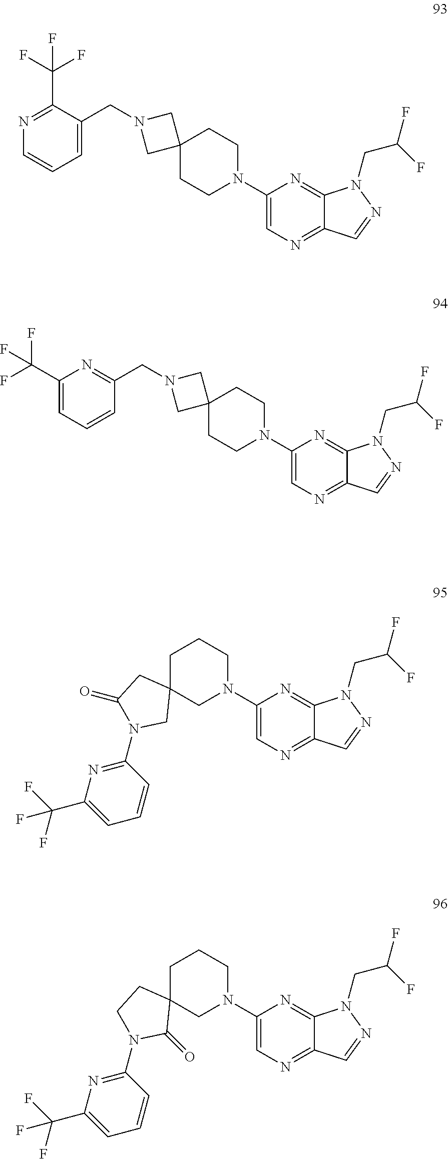 [SMILES: FC(F)Cn1ncc2ncc(N3CCC4(CC3)CN(Cc3cccnc3C(F)(F)F)C4)nc21 ; FC(F)Cn1ncc2ncc(N3CCC4(CC3)CN(Cc3cccc(C(F)(F)F)n3)C4)nc21 ; O=C1CC2(CCCN(c3cnc4cnn(CC(F)F)c4n3)C2)CN1c1cccc(C(F)(F)F)n1 ; O=C1N(c2cccc(C(F)(F)F)n2)CCC12CCCN(c1cnc3cnn(CC(F)F)c3n1)C2]

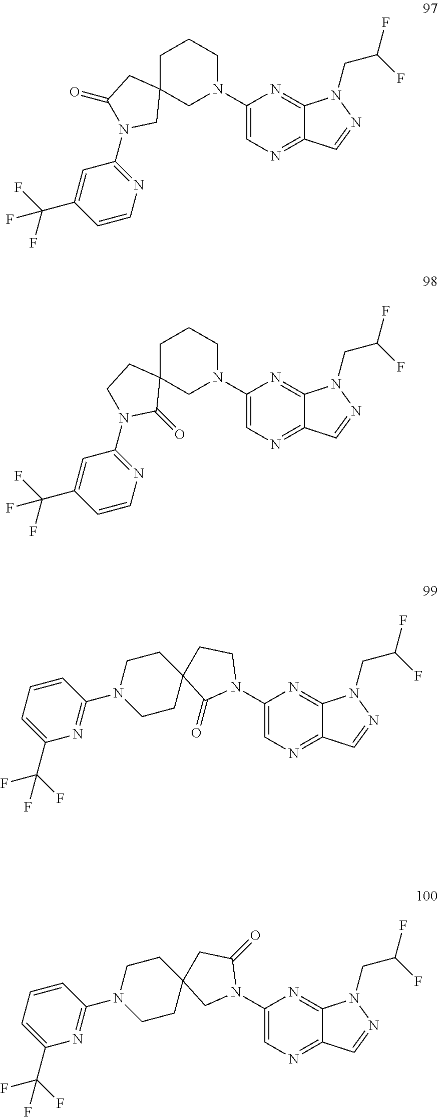 [SMILES: O=C1CC2(CCCN(c3cnc4cnn(CC(F)F)c4n3)C2)CN1c1cc(C(F)(F)F)ccn1 ; O=C1N(c2cc(C(F)(F)F)ccn2)CCC12CCCN(c1cnc3cnn(CC(F)F)c3n1)C2 ; O=C1N(c2cnc3cnn(CC(F)F)c3n2)CCC12CCN(c1cccc(C(F)(F)F)n1)CC2 ; O=C1CC2(CCN(c3cccc(C(F)(F)F)n3)CC2)CN1c1cnc2cnn(CC(F)F)c2n1]

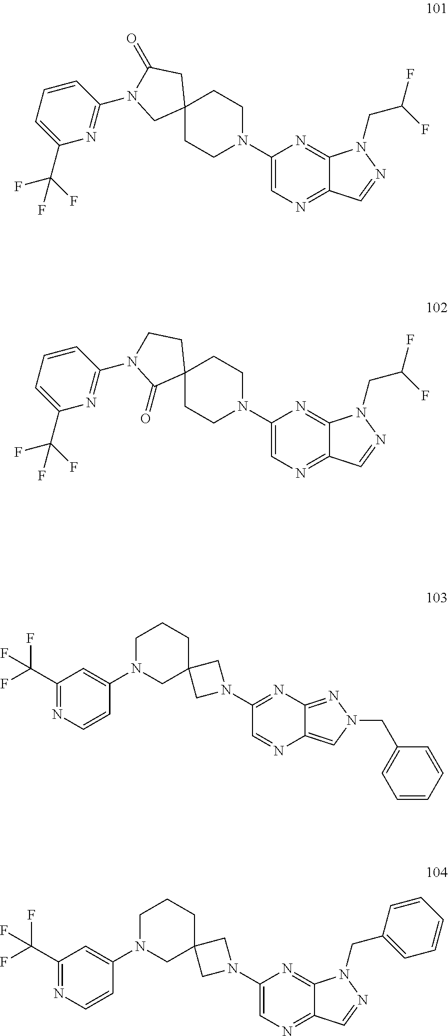 [SMILES: O=C1CC2(CCN(c3cnc4cnn(CC(F)F)c4n3)CC2)CN1c1cccc(C(F)(F)F)n1 ; O=C1N(c2cccc(C(F)(F)F)n2)CCC12CCN(c1cnc3cnn(CC(F)F)c3n1)CC2 ; FC(F)(F)c1cc(N2CCCC3(C2)CN(c2cnc4cn(Cc5ccccc5)nc4n2)C3)ccn1 ; FC(F)(F)c1cc(N2CCCC3(C2)CN(c2cnc4cnn(Cc5ccccc5)c4n2)C3)ccn1]

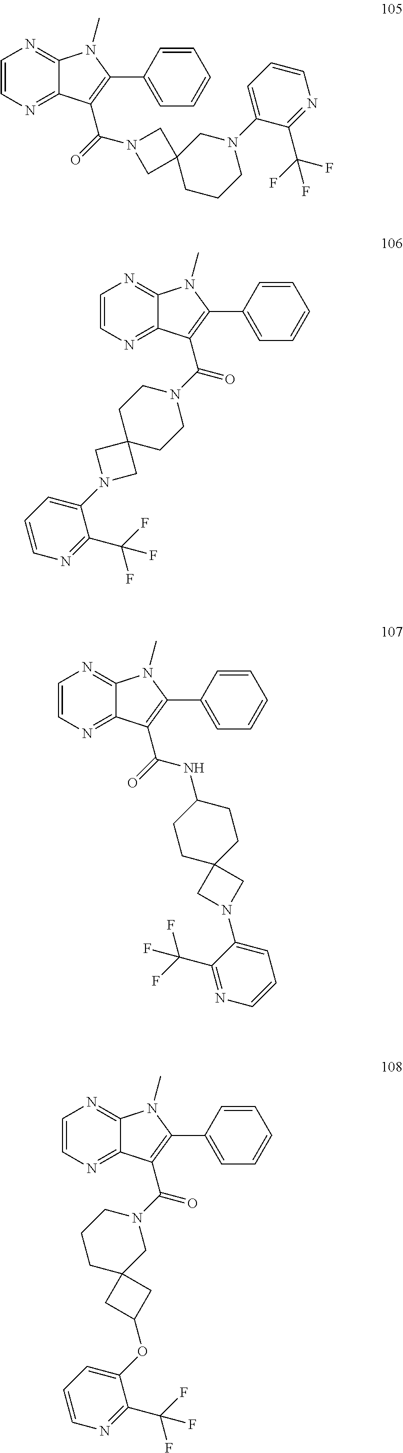 [SMILES: Cn1c(-c2ccccc2)c(C(=O)N2CC3(CCCN(c4cccnc4C(F)(F)F)C3)C2)c2nccnc21 ; Cn1c(-c2ccccc2)c(C(=O)N2CCC3(CC2)CN(c2cccnc2C(F)(F)F)C3)c2nccnc21 ; Cn1c(-c2ccccc2)c(C(=O)NC2CCC3(CC2)CN(c2cccnc2C(F)(F)F)C3)c2nccnc21 ; Cn1c(-c2ccccc2)c(C(=O)N2CCCC3(CC(Oc4cccnc4C(F)(F)F)C3)C2)c2nccnc21]

or a pharmaceutically acceptable salt thereof.

Clause 100. A pharmaceutical composition comprising a compound of any of clauses 1-99, or a pharmaceutically acceptable salt thereof, and a pharmaceutically acceptable excipient.

Clause 101. A kit comprising a compound of any of clauses 1-99, or a pharmaceutically acceptable salt thereof, or a pharmaceutical composition of clause 100, and instructions for administering the compound or pharmaceutical composition to a subject in need thereof.

Clause 102. A method of treating a disease or disorder in a subject in need thereof, the method comprising administering an effective amount of a compound of any of clauses 1-99, or pharmaceutically acceptable salt thereof, or a pharmaceutical composition of clause 100.

Clause 103. The method of clause 102, wherein the disease or disorder is associated with glucocerebrosidase activity.

Clause 104. The method of clause 102 or 103, wherein the disease or disorder is a neurological disease or disorder.

Clause 105. The method of clause 104, wherein the neurological disease or disorder is Parkinson's disease or Gaucher's disease.

Clause 106. A method of activating glucocerebrosidase, the method comprising contacting glucocerebrosidase with an effective amount of a compound of any of clauses 1-99, or pharmaceutically acceptable salt thereof, or a pharmaceutical composition of clause 100.

Clause 107. The method of clause 106, wherein the contacting is in vitro.

Clause 108. The method of clause 106, wherein the contacting is in vivo.

What is claimed is:

1. A compound of Formula (I): 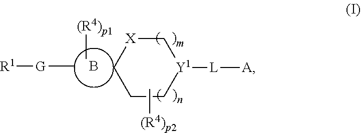
or a pharmaceutically acceptable salt thereof, wherein:
R¹ is substituted or unsubstituted heteroaryl, substituted or unsubstituted aryl, haloalkyl, or a nitrogen protecting group;
G is a bond, —NR^{A}—, —C(═O)—, —SO₂—, —O—, —OC(R⁵)(R⁶)—, or —C(R⁵)(R⁶)—;
B is cycloalkyl or heterocyclyl comprising at least one nitrogen atom in its ring;
each occurrence of R⁴ is independently halogen, substituted or unsubstituted alkyl, substituted or unsubstituted alkoxy, substituted or unsubstituted cycloalkyl, substituted or unsubstituted aryl, or two instances of R⁴ on different carbons combine to form a bridged ring, or two instances of R⁴ on the same carbon form with that carbon a carbonyl or a cycloalkyl;
X is —O—, —NR^{A}—, or —C(R⁴)₂—;
Y¹ is N or CR⁵;
each occurrence of R⁵ and R⁶ are each independently hydrogen, halogen, or substituted or unsubstituted alkyl;
L is a bond, —SO₂—, —C(═O)—, —C(═O)C(R⁵)(R⁶)—, —NR^{A}—, or —NHC(═O)—;
A is 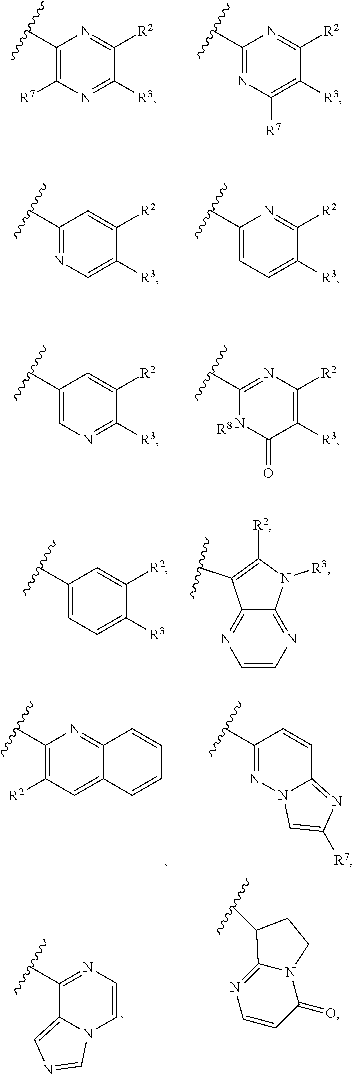
or substituted or unsubstituted 5-membered heteroaryl;
R² and R³ are each independently hydrogen, halogen, substituted or unsubstituted alkyl, substituted or unsubstituted alkoxy, substituted or unsubstituted carbocyclyl, substituted or unsubstituted heterocyclyl, substituted or unsubstituted aryl, or substituted or unsubstituted heteroaryl; or R² and R³ together with the atoms to which they are attached form a substituted or unsubstituted carbocyclyl, substituted or unsubstituted heterocyclyl, substituted or unsubstituted aryl, or substituted or unsubstituted heteroaryl;
R⁷ is hydrogen, substituted or unsubstituted alkyl, or substituted or unsubstituted alkoxy;
R⁸ is substituted or unsubstituted alkyl;
each R^{A} is independently hydrogen, substituted or unsubstituted alkyl, or a nitrogen protecting group;
m and n are each independently 0, 1, or 2; and
p1 and p2 are each independently 0, 1, 2, 3, 4, 5, or 6.

2. The compound of claim 1, or a pharmaceutically acceptable salt thereof, wherein:
R¹ is substituted or unsubstituted heteroaryl, or a nitrogen protecting group.

3. The compound of claim 1 or 2, or a pharmaceutically acceptable salt thereof, wherein:
R¹ is substituted or unsubstituted heteroaryl.

4. The compound of any of claims 1-3, or a pharmaceutically acceptable salt thereof, wherein:
R¹ is substituted or unsubstituted pyridinyl or substituted or unsubstituted pyrimidinyl.

5. The compound of any of claims 1-4, or a pharmaceutically acceptable salt thereof, wherein:
R¹ is substituted pyridinyl or substituted pyrimidinyl.

6. The compound of any of claims 1-5, or a pharmaceutically acceptable salt thereof, wherein:
R¹ is pyridinyl substituted with at least one instance of halogen, unsubstituted alkyl, or haloalkyl; or pyrimidinyl substituted with at least one instance of halogen, unsubstituted alkyl, or haloalkyl.

7. The compound of any of claims 1-6, or a pharmaceutically acceptable salt thereof, wherein:
R¹ is pyridinyl substituted with at least one instance of haloalkyl.

8. The compound of any of claims 1-7, or a pharmaceutically acceptable salt thereof, wherein:
R¹ is pyridinyl substituted with at least one instance of trifluoromethyl.

9. The compound of any of claims 1-6, or a pharmaceutically acceptable salt thereof, wherein:
R¹ is pyrimidinyl substituted with at least one instance of unsubstituted alkyl or haloalkyl.

10. The compound of any of claims 1-7, or a pharmaceutically acceptable salt thereof, wherein:
R¹ is pyrimidinyl substituted with at least one instance of methyl or trifluoromethyl.

11. The compound of claim 1, or a pharmaceutically acceptable salt thereof, wherein:
R¹ is substituted or unsubstituted phenyl.

12. The compound of any of claims 1 or 9, or a pharmaceutically acceptable salt thereof, wherein:
R¹ is unsubstituted phenyl or phenyl substituted with halogen or haloalkyl.

13. The compound of any of claims 1-12, or a pharmaceutically acceptable salt thereof, wherein:
R¹ is

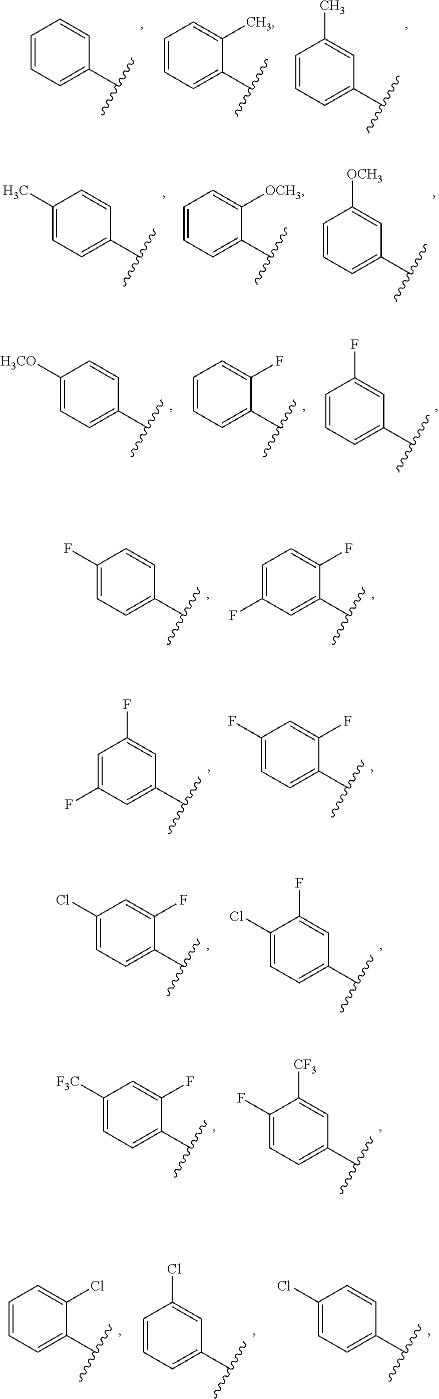

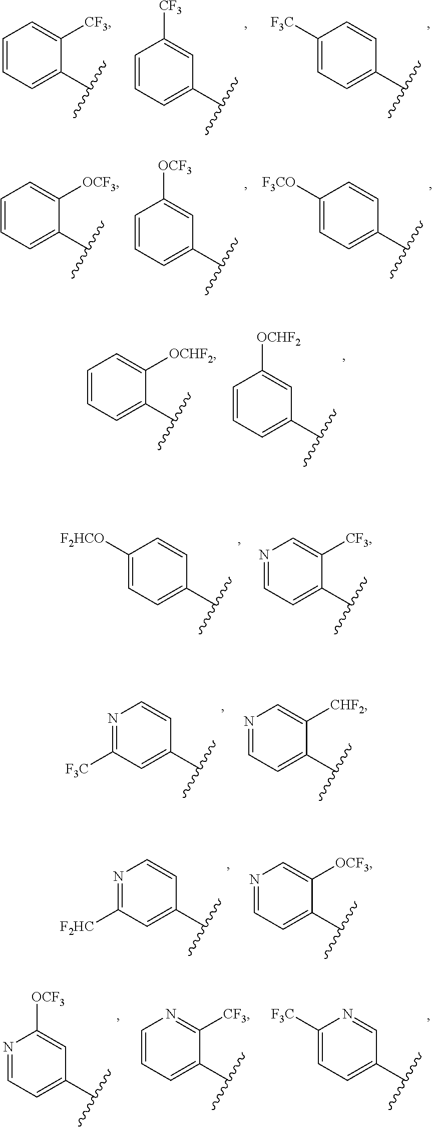

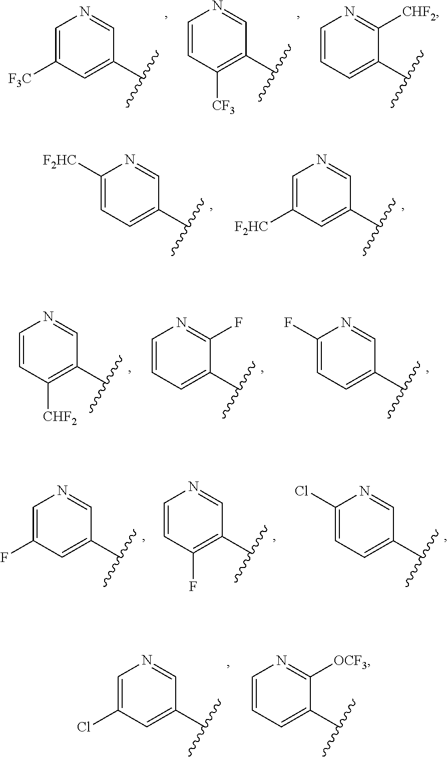

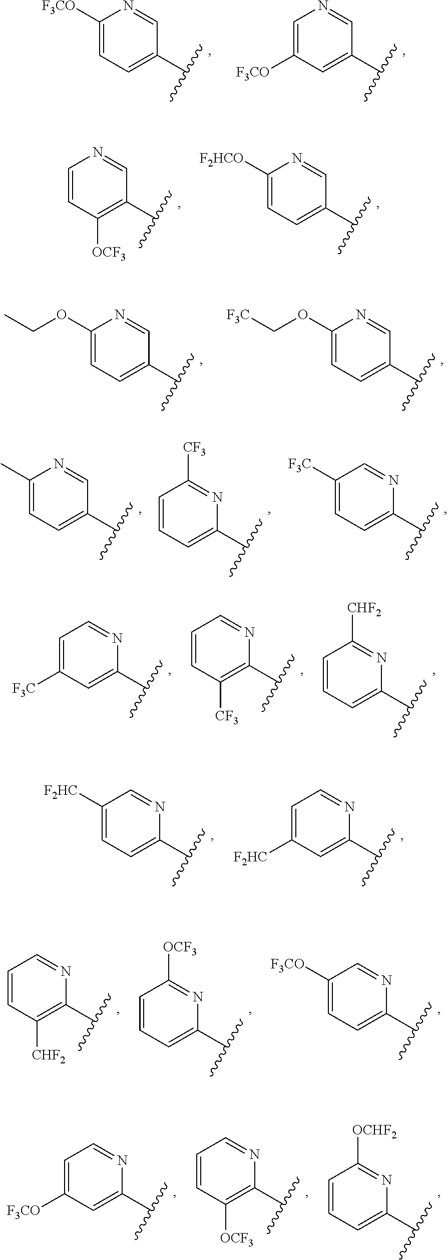

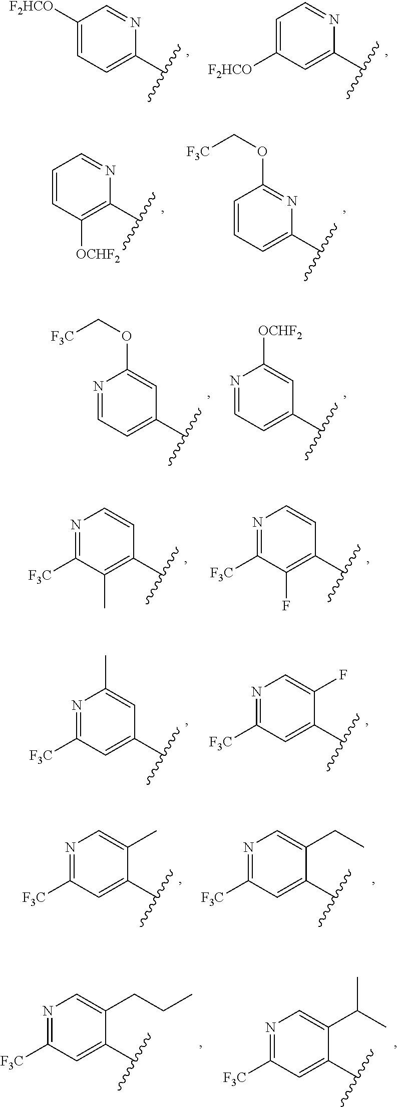

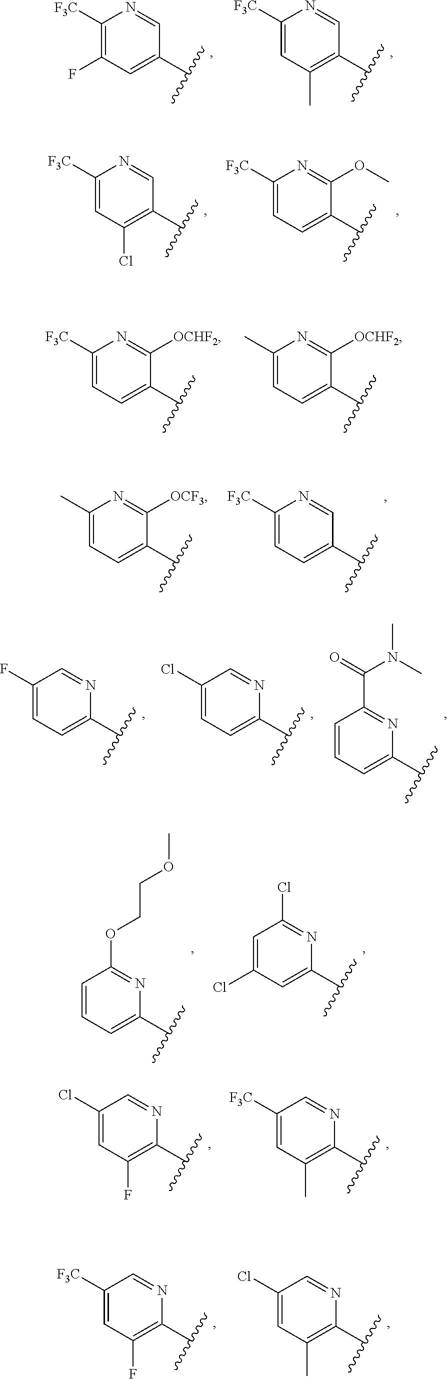

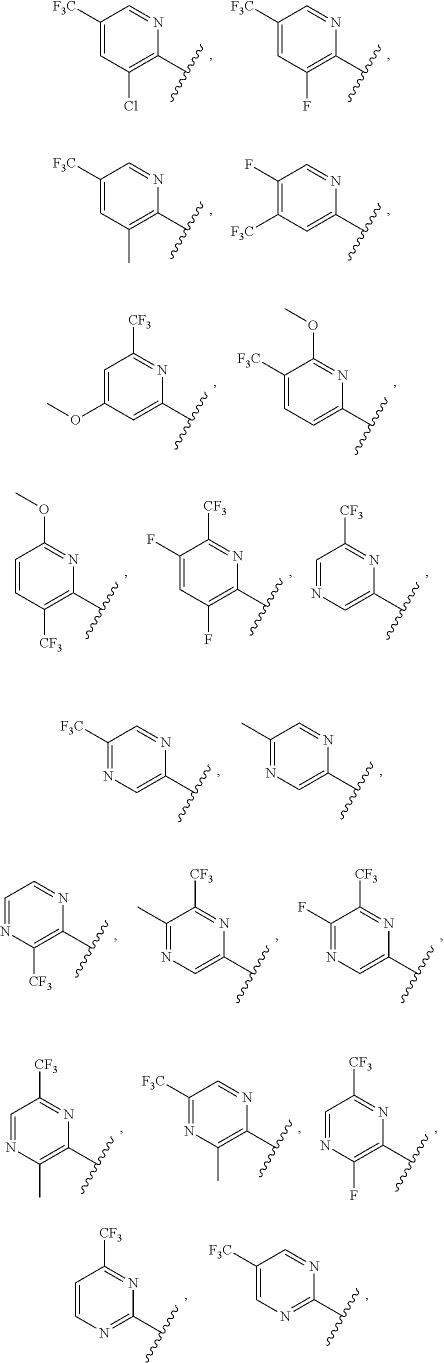

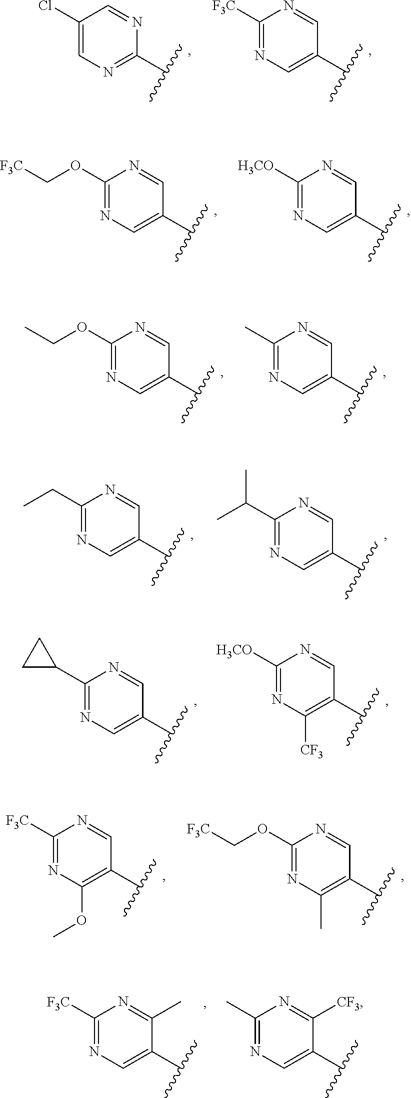

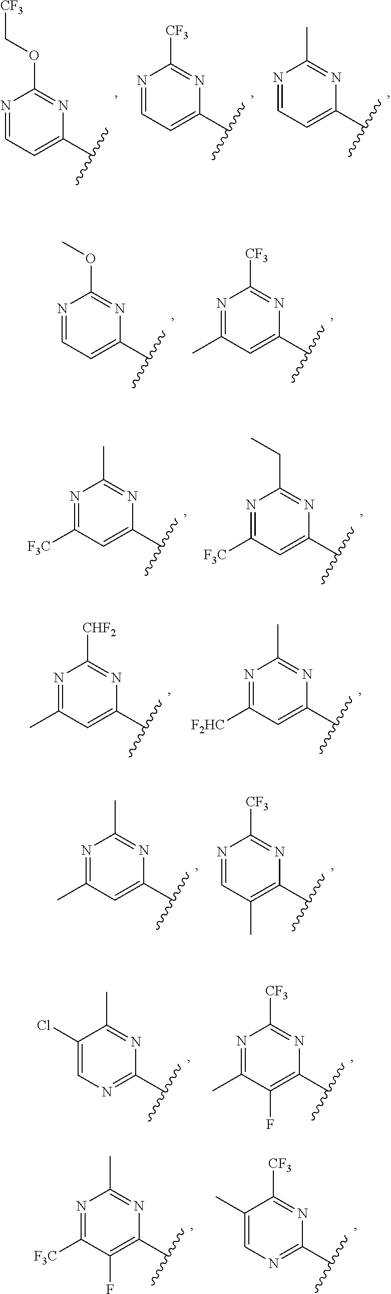

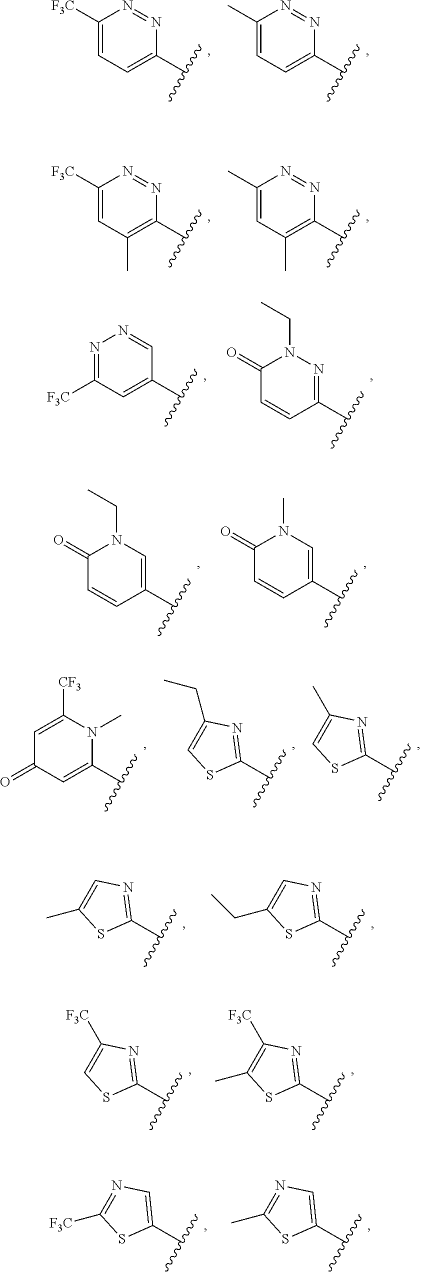

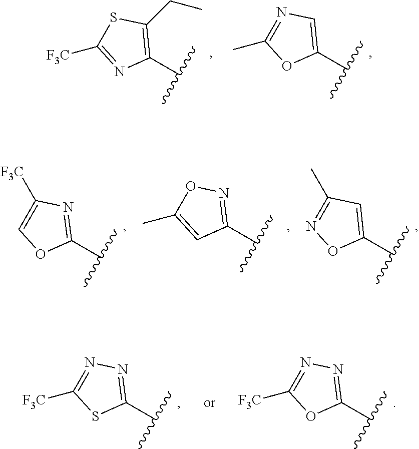

14. The compound of any of claims 1-13, or a pharmaceutically acceptable salt thereof, wherein:
R¹ is

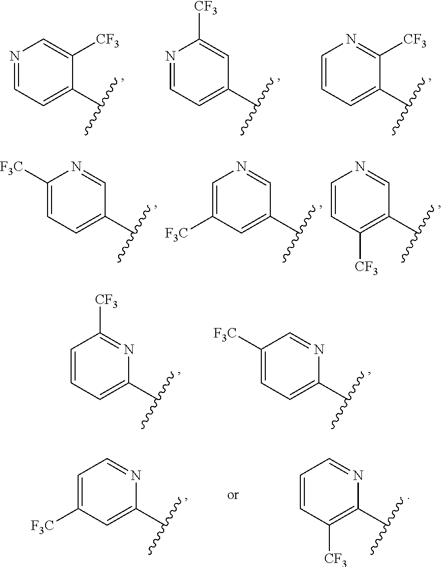

15. The compound of any of claims 1-13, or a pharmaceutically acceptable salt thereof, wherein:

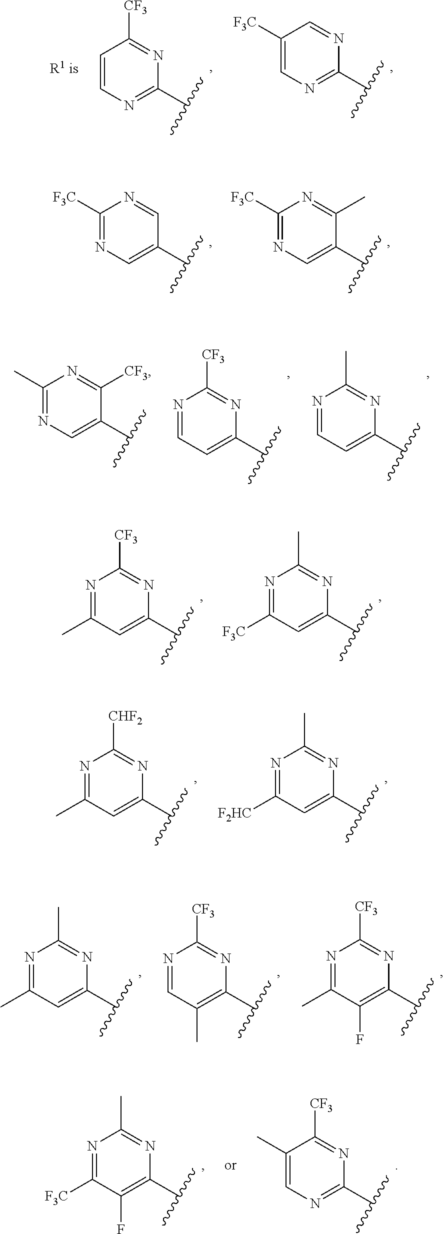

16. The compound of any of claims 1-13, or a pharmaceutically acceptable salt thereof, wherein:

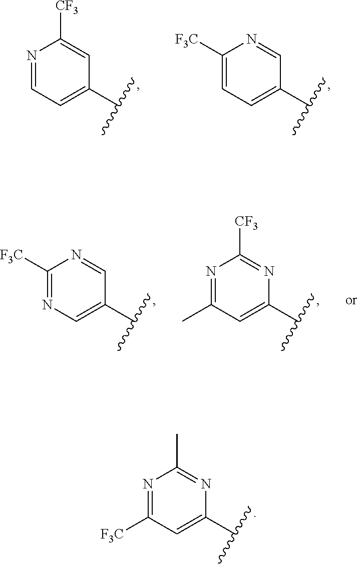 [SMILES: *c1ccnc(C(F)(F)F)c1 ; *c1ccc(C(F)(F)F)nc1 ; *c1cnc(C(F)(F)F)nc1 ; *c1cc(C)nc(C(F)(F)F)n1 ; *c1cc(C(F)(F)F)nc(C)n1]

17. The compound of any of claims 1 or 2, or a pharmaceutically acceptable salt thereof, wherein:
R¹ is a nitrogen protecting group (e.g.,

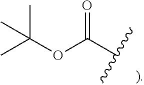 [SMILES: *C(=O)OC(C)(C)C]

18. The compound of any of claims 1-17, or a pharmaceutically acceptable salt thereof, wherein:
A is

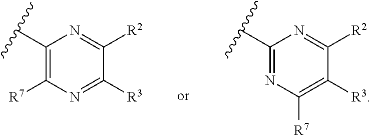

19. The compound of any of claims 1-18, or a pharmaceutically acceptable salt thereof, wherein:
A is

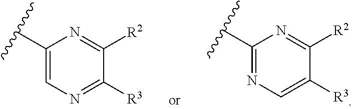

20. The compound of any of claims 1-19, or a pharmaceutically acceptable salt thereof, wherein:
R² and R³ are each independently hydrogen or substituted or unsubstituted heteroaryl; or R² and R³ together with the atoms to which they are attached form a substituted or unsubstituted aryl, substituted or unsubstituted heteroaryl, or substituted or unsubstituted heterocyclyl.

21. The compound of any of claims 1-20, or a pharmaceutically acceptable salt thereof, wherein:
R² and R³ together with the atoms to which they are attached form a substituted or unsubstituted heteroaryl.

22. The compound of any of claims 1-21, or a pharmaceutically acceptable salt thereof, wherein:
R² and R³ together with the atoms to which they are attached form a substituted or unsubstituted pyrazolyl.

23. The compound of any of claims 1-20, or a pharmaceutically acceptable salt thereof, wherein:
R² and R³ together with the atoms to which they are attached form a substituted or unsubstituted heterocyclyl.

24. The compound of any of claims 1-20, or a pharmaceutically acceptable salt thereof, wherein:
R² and R³ together with the atoms to which they are attached form a substituted or unsubstituted dihydroimidazol-2-one.

25. The compound of any of claims 1-22, or a pharmaceutically acceptable salt thereof, wherein:
A is 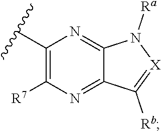
wherein R⁷ is hydrogen, unsubstituted C₁₋₄ alkyl, or unsubstituted C₁₋₄ alkoxy; X is N or CR^{a}; each R^{a} is independently hydrogen, substituted or unsubstituted alkyl, substituted or unsubstituted aryl, or substituted or unsubstituted heterocyclyl; and R^{b} is hydrogen, substituted or unsubstituted alkyl, or substituted or unsubstituted alkoxy.

26. The compound of any of claims 1-22, or a pharmaceutically acceptable salt thereof, wherein:
A is 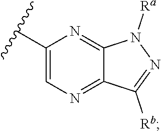
wherein R^{a} is hydrogen, substituted or unsubstituted alkyl, or substituted or unsubstituted heterocyclyl; and R^{b} is hydrogen, substituted or unsubstituted alkyl, or substituted or unsubstituted alkoxy.

27. The compound of any of claims 1-22, or a pharmaceutically acceptable salt thereof, wherein:
A is 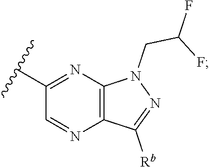
and R^{b} is hydrogen, substituted or unsubstituted alkyl, or substituted or unsubstituted alkoxy.

28. The compound of any of claims 1-22, or a pharmaceutically acceptable salt thereof, wherein:
A is 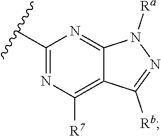
R⁷ is hydrogen, unsubstituted C₁₋₄ alkyl, or unsubstituted C₁₋₄ alkoxy; R^{a} is substituted or unsubstituted alkyl or substituted or unsubstituted heterocyclyl; and R^{b} is hydrogen, substituted or unsubstituted alkyl, or substituted or unsubstituted alkoxy.

29. The compound of any of claims 1-22, or a pharmaceutically acceptable salt thereof, wherein:
A is 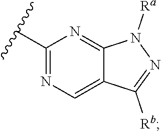
R^{a} is substituted or unsubstituted alkyl or substituted or unsubstituted heterocyclyl; and R^{b} is hydrogen, substituted or unsubstituted alkyl, or substituted or unsubstituted alkoxy.

30. The compound of any of claims 1-22, or a pharmaceutically acceptable salt thereof, wherein:
A is 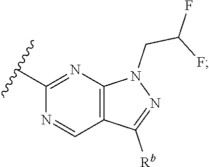
and R^{b} is hydrogen, substituted or unsubstituted alkyl, or substituted or unsubstituted alkoxy.

31. The compound of any of claims 25-30, or a pharmaceutically acceptable salt thereof, wherein:
R^{b} is substituted or unsubstituted alkyl, or substituted or unsubstituted alkoxy.

32. The compound of any of claims 25-30, or a pharmaceutically acceptable salt thereof, wherein:
R^{b} is hydrogen.

33. The compound of any of claims 1-20, 23, or 24, or a pharmaceutically acceptable salt thereof, wherein:
A is 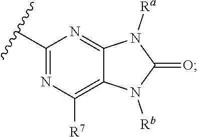
wherein R⁷ is hydrogen, unsubstituted C₁₋₄ alkyl, or unsubstituted C₁₋₄ alkoxy; R^{a} is substituted or unsubstituted alkyl or substituted or unsubstituted heterocyclyl; and R^{b} is substituted or unsubstituted alkyl or substituted or unsubstituted heterocyclyl.

34. The compound of any of claims 1-20, 23, or 24, or a pharmaceutically acceptable salt thereof, wherein:
A is 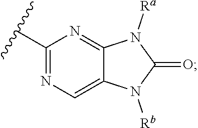
wherein R^{a} is substituted or unsubstituted alkyl or substituted or unsubstituted heterocyclyl; and R^{b} is substituted or unsubstituted alkyl or substituted or unsubstituted heterocyclyl.

35. The compound of any of claims 1-20, or a pharmaceutically acceptable salt thereof, wherein:
A is

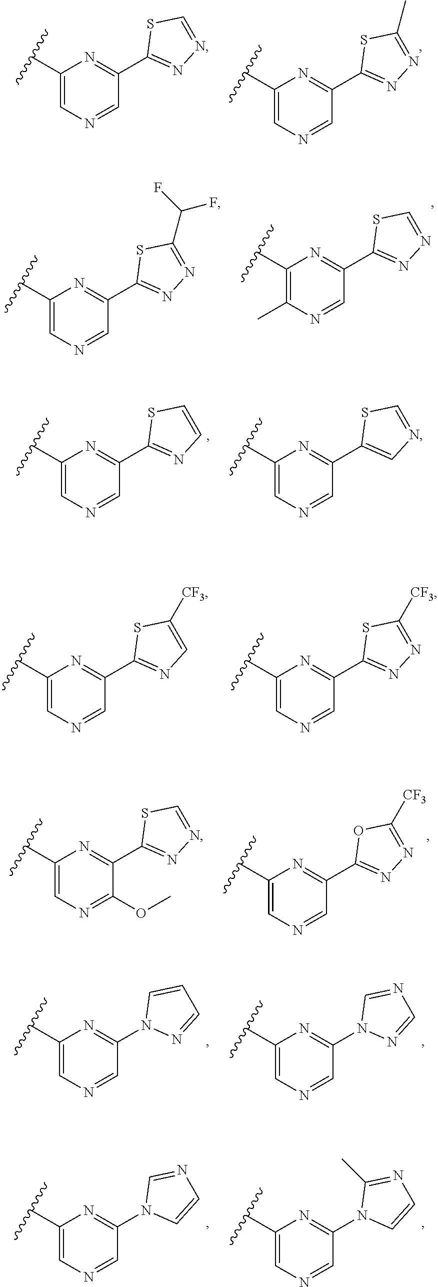

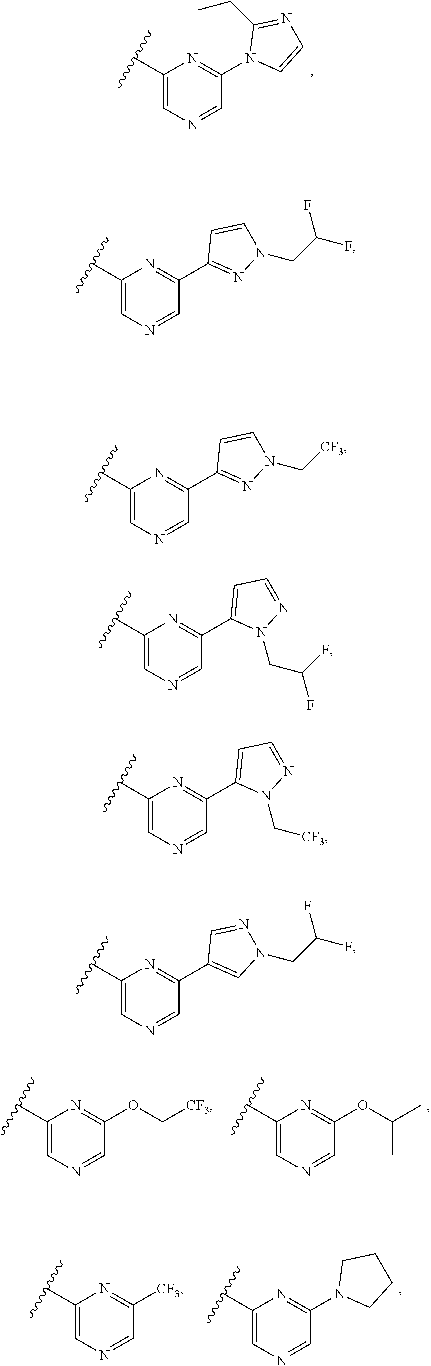

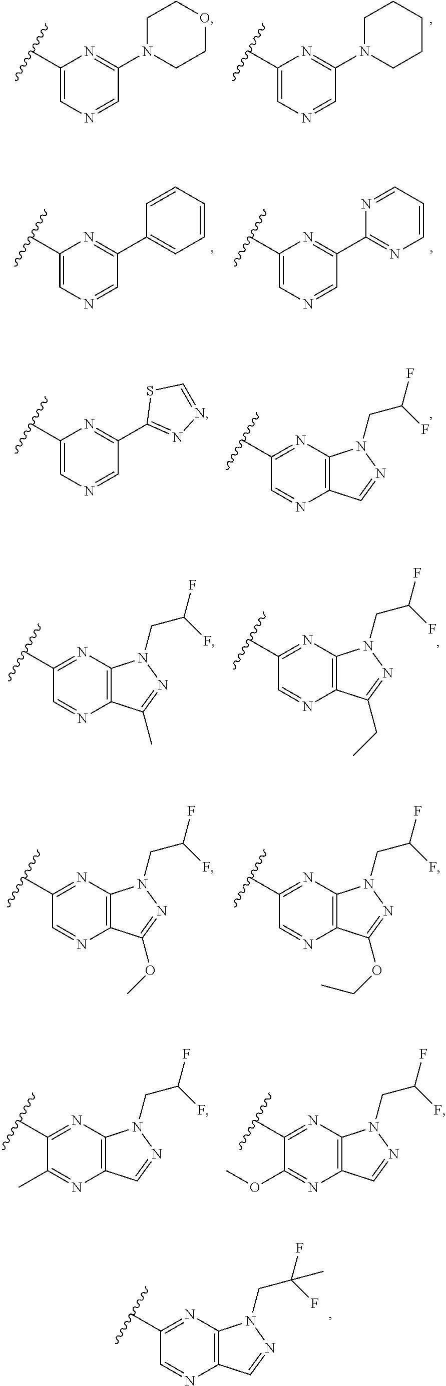

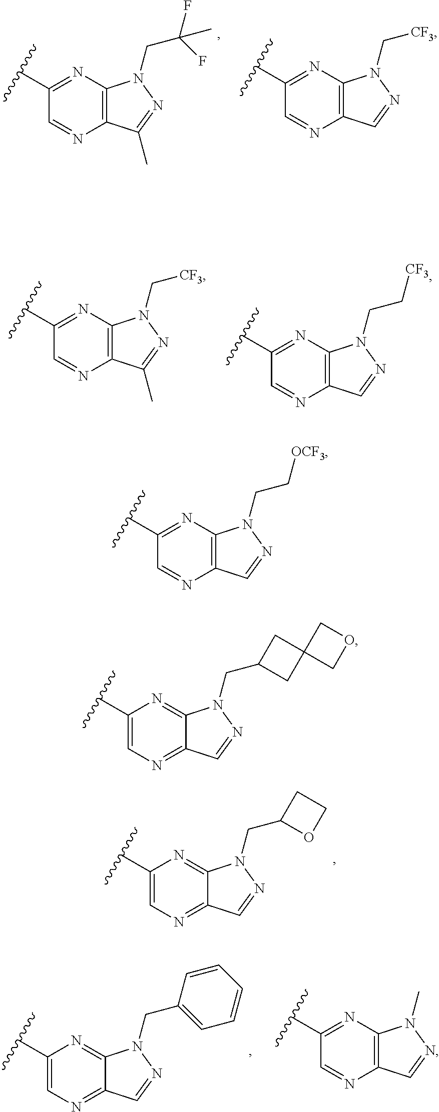

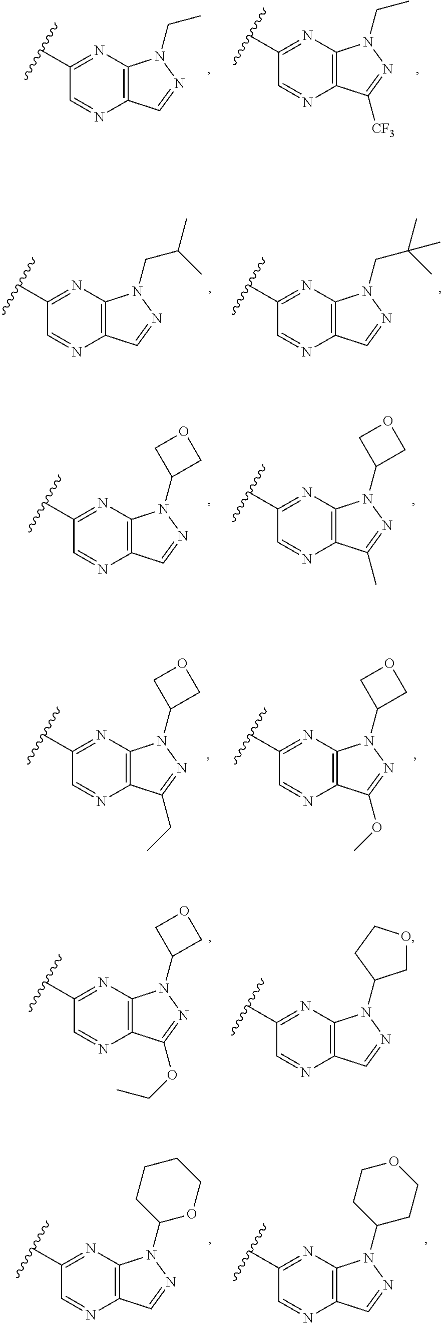

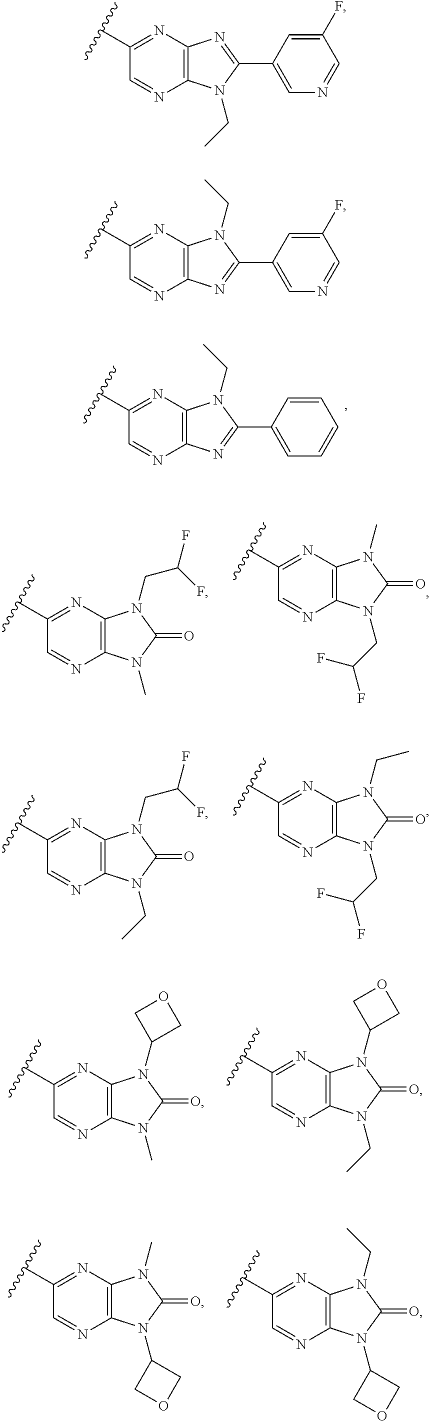

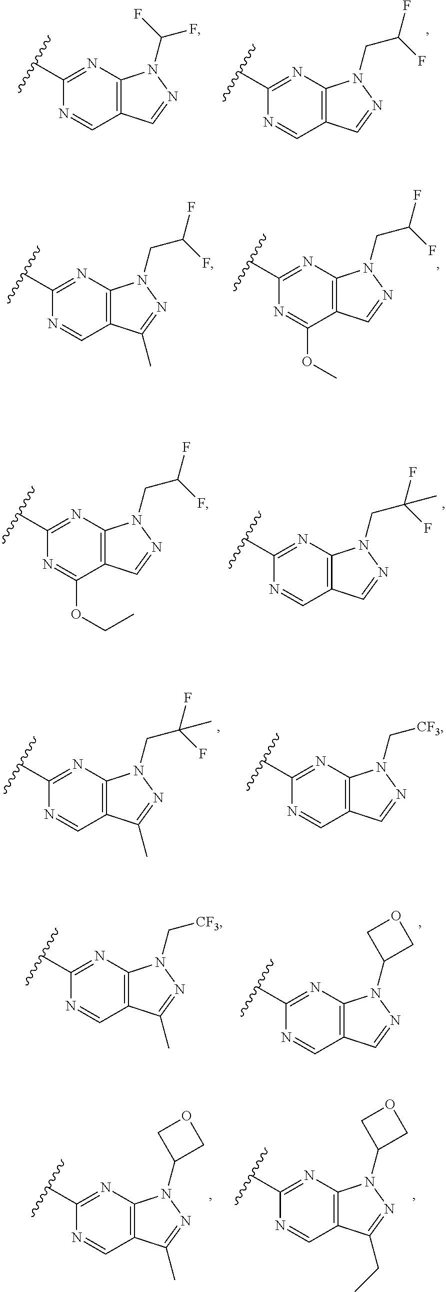

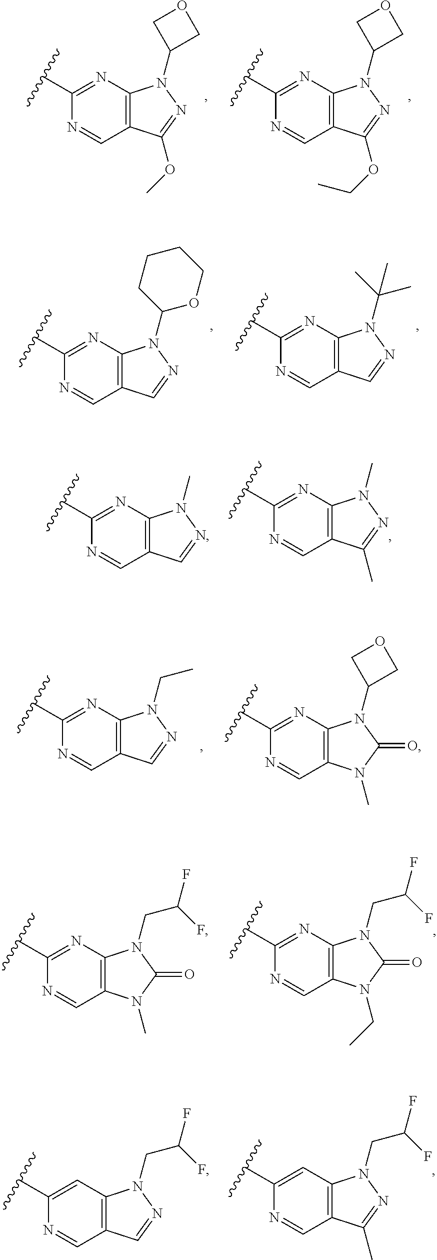

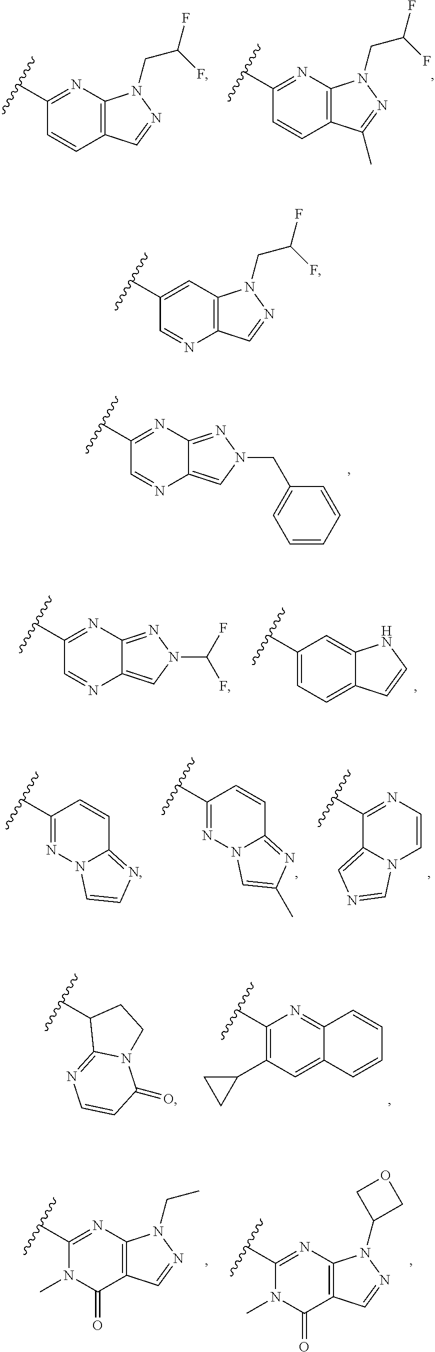

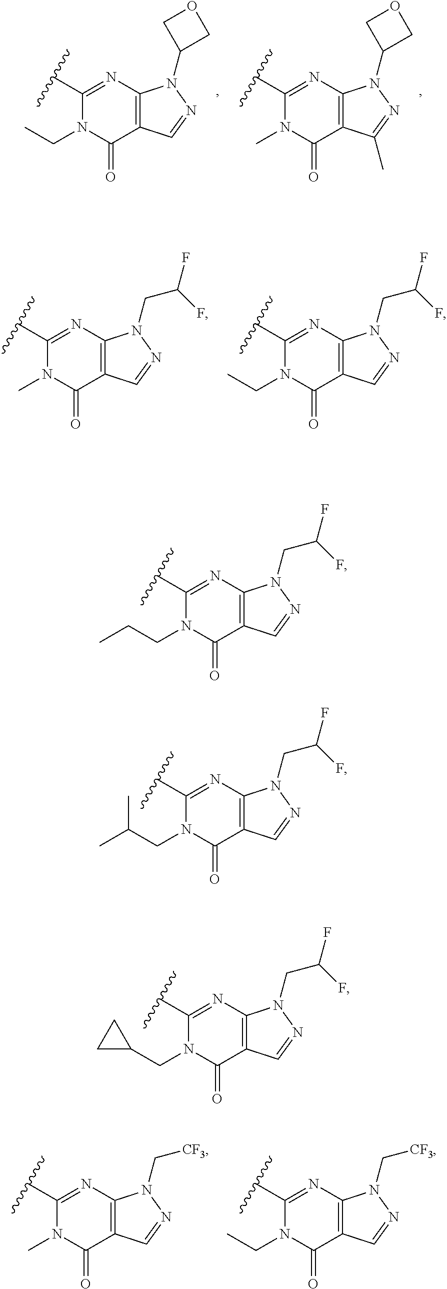

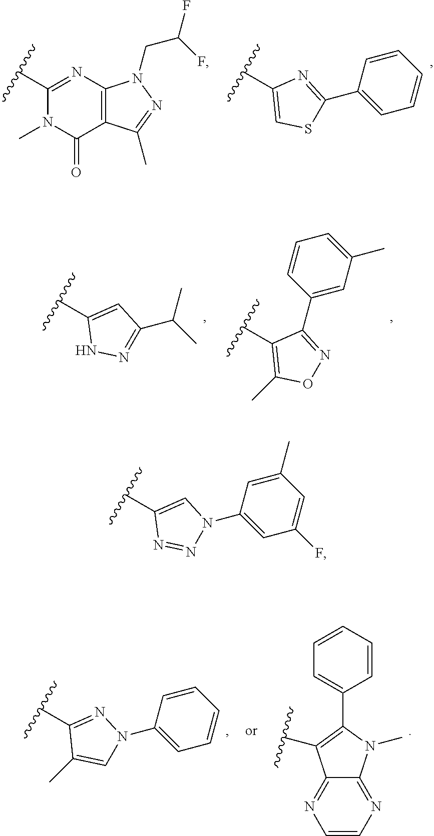

36. The compound of any of claims 1-23, or a pharmaceutically acceptable salt thereof, wherein:

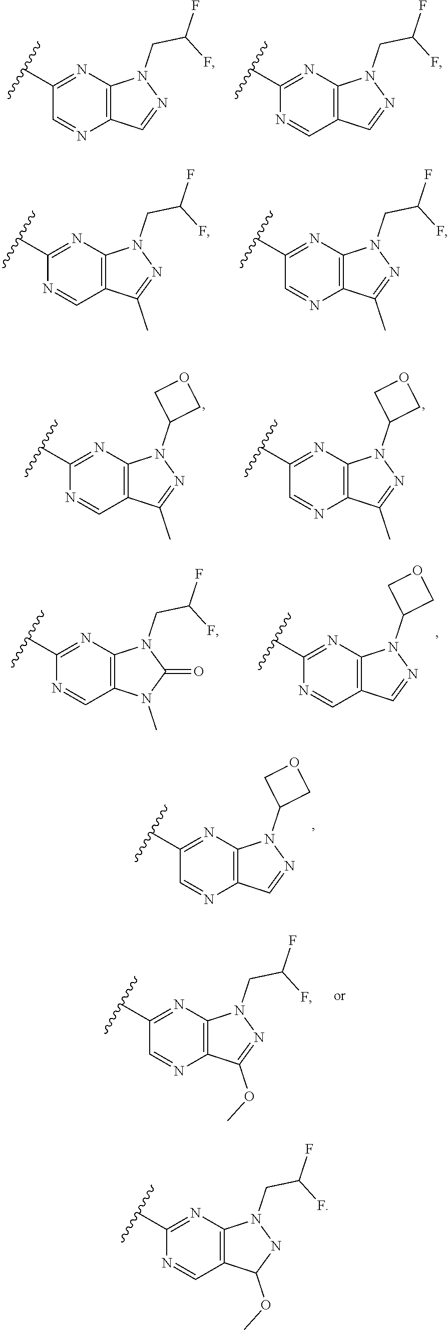

37. The compound of any of claims 1-23, or a pharmaceutically acceptable salt thereof, wherein:
A is

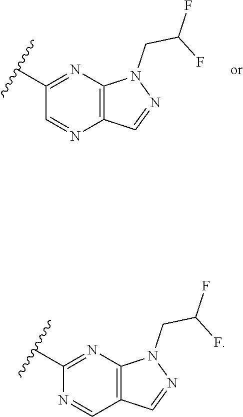 [SMILES: *c1cnc2cnn(CC(F)F)c2n1 ; *c1ncc2cnn(CC(F)F)c2n1]

38. The compound of any of claims 1-37, or a pharmaceutically acceptable salt thereof, wherein:
G is a bond or —O—.

39. The compound of any of claims 1-38, or a pharmaceutically acceptable salt thereof, wherein:
G is a band.

40. The compound of any of claims 1-39, or a pharmaceutically acceptable salt thereof, wherein:
Y¹ is N.

41. The compound of any of claims 1-39, or a pharmaceutically acceptable salt thereof, wherein:
Y¹ is CR⁵.

42. The compound of any of claims 1-41, or a pharmaceutically acceptable salt thereof, wherein:
m is 1; and n is 1.

43. The compound of any of claims 1-41, or a pharmaceutically acceptable salt thereof, wherein:
m is 0; and n is 0.

44. The compound of any of claims 1-41, or a pharmaceutically acceptable salt thereof, wherein:
m is 2; and n is 0.

45. The compound of any of claims 1-41, or a pharmaceutically acceptable salt thereof, wherein:
m is 1; and n is 0.

46. The compound of any of claims 1-41, or a pharmaceutically acceptable salt thereof, wherein:
m is 0; and n is 1.

47. The compound of any of claims 1-41, or a pharmaceutically acceptable salt thereof, wherein:
m is 0; and n is 2.

48. The compound of any of claims 1-47, or a pharmaceutically acceptable salt thereof, wherein:
L is a bond.

49. The compound of any of claims 1-48, or a pharmaceutically acceptable salt thereof, wherein:
—(R⁴)ₚ₁— is 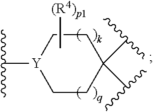
wherein:
Y is N or CR⁵;
k and q are each independently 0, 1, or 2; and Y is bound to G of Formula (I).

50. The compound of claim 49, or a pharmaceutically acceptable salt thereof, wherein:
Y is N.

51. The compound of claim 49, or a pharmaceutically acceptable salt thereof, wherein:
Y is CR⁵.

52. The compound of any of claims 49-51, or a pharmaceutically acceptable salt thereof, wherein:
k is 0; and q is 2.

53. The compound of any of claims 49-51, or a pharmaceutically acceptable salt thereof, wherein:
k is 1; and q is 1.

54. The compound of any of claims 49-51, or a pharmaceutically acceptable salt thereof, wherein:
k is 0; and q is 0.

55. The compound of any of claims 49-51, or a pharmaceutically acceptable salt thereof, wherein:
k is 2; and q is 0.

56. The compound of any of claims 49-51, or a pharmaceutically acceptable salt thereof, wherein:
k is 1; and q is 0.

57. The compound of any of claims 49-51, or a pharmaceutically acceptable salt thereof, wherein:
k is 0; and q is 1.

58. The compound of any of claims 1-57, or a pharmaceutically acceptable salt thereof, wherein:
—B(R⁴)^{p1}— is

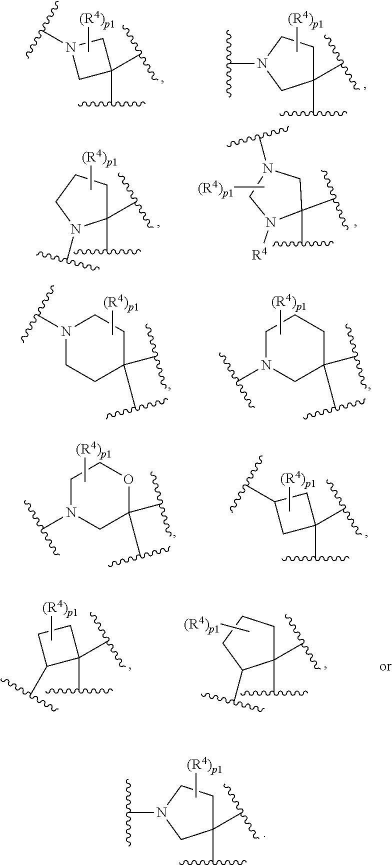

59. The compound of any of claims 1-58, or a pharmaceutically acceptable salt thereof, wherein:
—B(R⁴)^{p1}— is

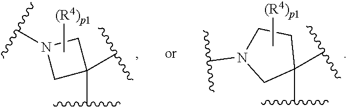

60. The compound of any of claims 1-58, or a pharmaceutically acceptable salt thereof, wherein:
—B(R⁴)^{p1}— is

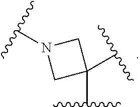 [SMILES: *N1CC(*)(*)C1]

61. The compound of any of claims 1-58, or a pharmaceutically acceptable salt thereof, wherein:
—B(R⁴)^{p1}— is

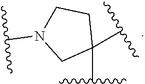 [SMILES: *N1CCC(*)(*)C1]

62. The compound of claim 1, wherein the compound is of formula (I-l): 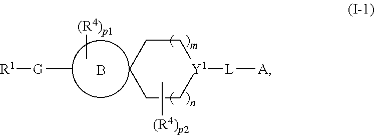
or a pharmaceutically acceptable salt thereof.

63. The compound of claim 1, wherein the compound is of formula (I-a): 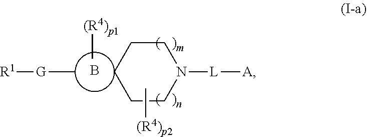
or a pharmaceutically acceptable salt thereof.

64. The compound of claim 1 or 52, wherein the compound is of formula (I-b): 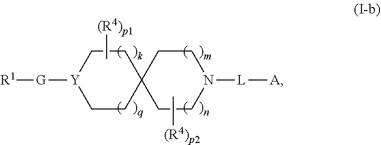
or a pharmaceutically acceptable salt thereof, wherein:
Y is N or CR⁵; and
k and q are each independently 0, 1, or 2.

65. The compound of claim 1, wherein the compound is of formula (I-g): 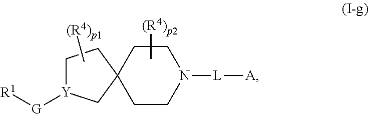
or a pharmaceutically acceptable salt thereof.

66. The compound of claim 1, wherein the compound is of formula (I-g-13): 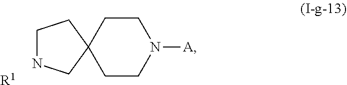
or a pharmaceutically acceptable salt thereof.

67. The compound of claim 1, wherein the compound is of formula (I-k): 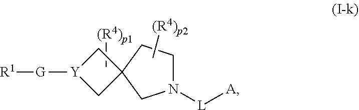
or a pharmaceutically acceptable salt thereof.

68. The compound of claim 1, wherein the compound is of formula (I-k-3): 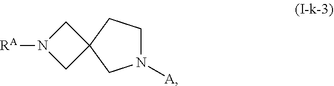
or a pharmaceutically acceptable salt thereof.

69. The compound of claim 1, wherein the compound is of formula (I-k-5): 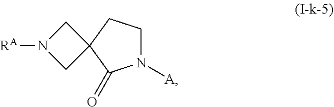
or a pharmaceutically acceptable salt thereof.

70. The compound of claim 1, wherein the compound is of formula (I-l): 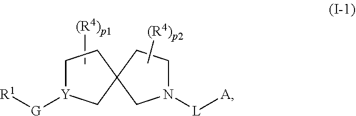
or a pharmaceutically acceptable salt thereof.

71. The compound of claim 1, wherein the compound is of formula (I-l-3): 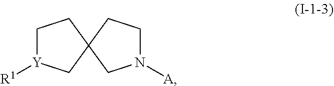
or a pharmaceutically acceptable salt thereof.

72. The compound of claim 1, wherein the compound is of formula (I-l-5): 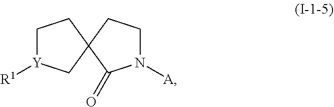
or a pharmaceutically acceptable salt thereof.

73. The compound of claim 1, wherein the compound is of formula (I-m): 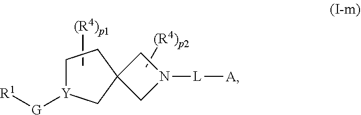
or a pharmaceutically acceptable salt thereof.

74. The compound of claim 1, wherein the compound is of formula (I-m-3): 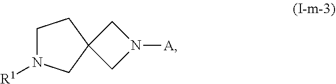
or a pharmaceutically acceptable salt thereof.

75. The compound of claim 1, wherein the compound is of formula: 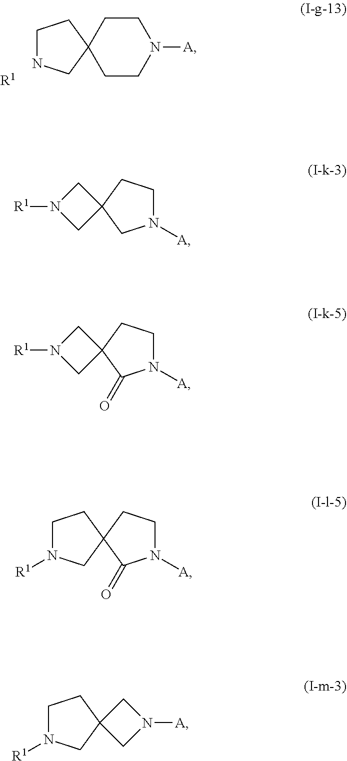
or a pharmaceutically acceptable salt thereof.

76. The compound of claim 1, wherein the compound is of Formula (I-n): 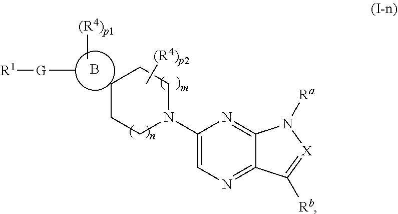
or a pharmaceutically acceptable salt thereof; wherein:
X is N or CR^{a}; each R^{a} is independently hydrogen, substituted or unsubstituted alkyl, substituted or unsubstituted heterocyclyl, or substituted or unsubstituted aryl; and R^{b} is hydrogen, substituted or unsubstituted alkyl, or substituted or unsubstituted alkoxy.

77. The compound of claim 1, wherein the compound is of Formula (I-n′): 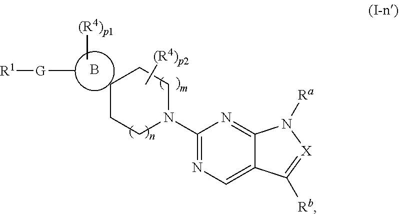
or a pharmaceutically acceptable salt thereof; wherein:
X is N or CR^{a}; each R^{a} is independently hydrogen, substituted or unsubstituted alkyl, substituted or unsubstituted heterocyclyl, or substituted or unsubstituted aryl; and R^{b} is hydrogen, substituted or unsubstituted alkyl, or substituted or unsubstituted alkoxy.

78. The compound of claim 1, wherein the compound is of Formula (I-o): 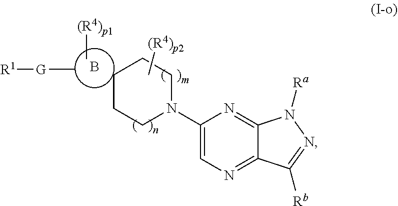
or a pharmaceutically acceptable salt thereof; wherein:
R^{a} is independently hydrogen, substituted or unsubstituted alkyl, or substituted or unsubstituted heterocyclyl; and R^{b} is hydrogen, substituted or unsubstituted alkyl, or substituted or unsubstituted alkoxy.

79. The compound of claim 1, wherein the compound is of Formula (I-o′): 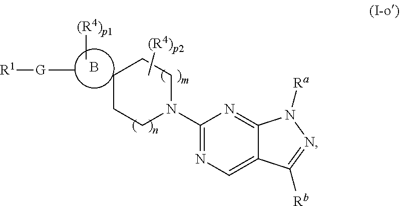
or a pharmaceutically acceptable salt thereof; wherein:
R^{a} is independently hydrogen, substituted or unsubstituted alkyl, or substituted or unsubstituted heterocyclyl; and R^{b} is hydrogen, substituted or unsubstituted alkyl, or substituted or unsubstituted alkoxy.

80. The compound of claim 1, wherein the compound is of Formula (I-p): 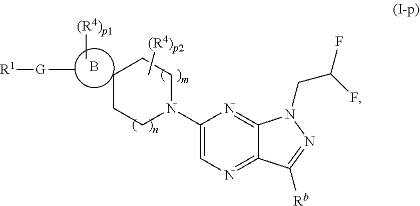
or a pharmaceutically acceptable salt thereof; wherein R^{b} is hydrogen, substituted or unsubstituted alkyl, or substituted or unsubstituted alkoxy.

81. The compound of claim 1, wherein the compound is of Formula (I-p′): 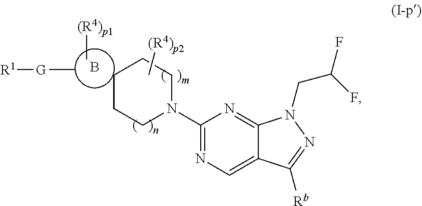
or a pharmaceutically acceptable salt thereof; wherein R^{b} is hydrogen, substituted or unsubstituted alkyl, or substituted or unsubstituted alkoxy.

82. The compound of claim 1, wherein the compound is of Formula (I-x): 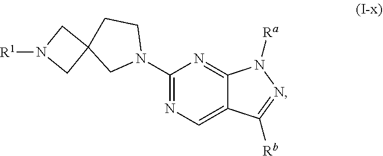
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

83. The compound of claim 1, wherein the compound is of Formula (I-x-1): 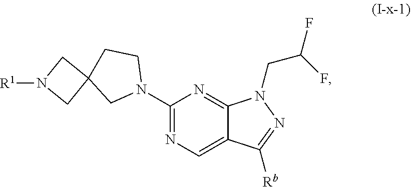
or a pharmaceutically acceptable salt thereof; wherein R^{b} is hydrogen, substituted or unsubstituted alkyl, or substituted or unsubstituted alkoxy.

84. The compound of claim 1, wherein the compound is of Formula (I-y): 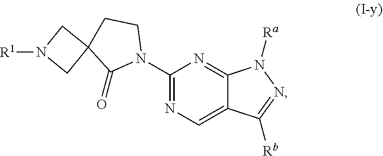
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

85. The compound of claim 1, wherein the compound is of Formula (I-z): 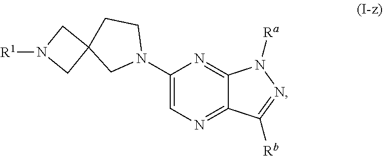
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

86. The compound of claim 1, wherein the compound is of Formula (I-aa): 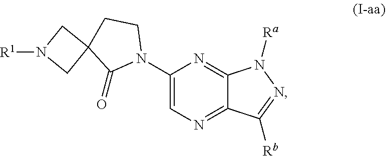
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

87. The compound of claim 1, wherein the compound is of Formula (I-bb): 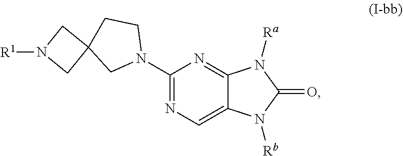
or a pharmaceutically acceptable salt thereof; wherein R^{a} is substituted or unsubstituted alkyl or substituted or unsubstituted heterocyclyl; and R^{b} is substituted or unsubstituted alkyl or substituted or unsubstituted heterocyclyl.

88. The compound of claim 1, wherein the compound is of Formula (I-cc): 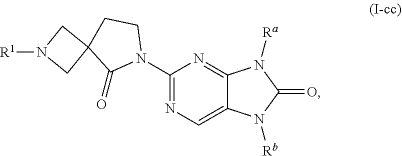
or a pharmaceutically acceptable salt thereof; wherein R^{a} is substituted or unsubstituted alkyl or substituted or unsubstituted heterocyclyl; and R^{b} is substituted or unsubstituted alkyl or substituted or unsubstituted heterocyclyl.

89. The compound of claim 1, wherein the compound is of Formula (I-dd): 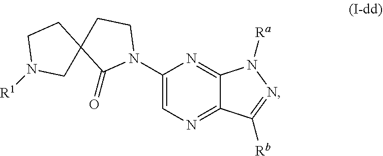
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

90. The compound of claim 1, wherein the compound is of Formula (I-ee): 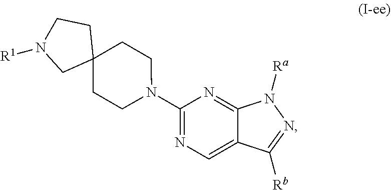
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

91. The compound of claim 1, wherein the compound is of Formula (I-ff): 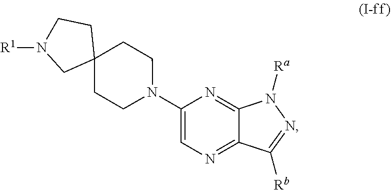
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

92. The compound of claim 1, wherein the compound is of Formula (I-gg): 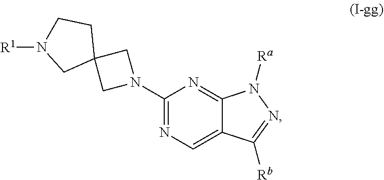
or a pharmaceutically acceptable salt thereof; wherein R^{a} is substituted or unsubstituted alkyl or substituted or unsubstituted heterocyclyl; and R^{b} is hydrogen, substituted or unsubstituted alkyl, or substituted or unsubstituted alkoxy.

93. The compound of claim 1, wherein the compound is a compound of Table 1, or a pharmaceutically acceptable salt thereof.

94. A pharmaceutical composition comprising a compound of any of claims 1-91, or a pharmaceutically acceptable salt thereof, and a pharmaceutically acceptable excipient.

95. A kit comprising a compound of any of claims 1-91, or a pharmaceutically acceptable salt thereof, or a pharmaceutical composition of claim 92, and instructions for administering the compound or pharmaceutical composition to a subject in need thereof.

96. A method of treating a disease or disorder in a subject in need thereof, the method comprising administering an effective amount of a compound of any of claims 1-91, or pharmaceutically acceptable salt thereof, or a pharmaceutical composition of claim 92.

97. The method of claim 94, wherein the disease or disorder is associated with glucocerebrosidase activity.

98. The method of claim 94 or 95, wherein the disease or disorder is a neurological disease or disorder.

99. The method of claim 96, wherein the neurological disease or disorder is Parkinson's disease or Gaucher's disease.

100. A method of activating glucocerebrosidase, the method comprising contacting glucocerebrosidase with an effective amount of a compound of any of claims 1-91, or pharmaceutically acceptable salt thereof, or a pharmaceutical composition of claim 92.

101. The method of claim 98, wherein the contacting is in vitro.

102. The method of claim 98, wherein the contacting is in vivo.
